(19) United States
(12) Patent Application Publication
(10) Pub. No.: US 2024/0400591 A1
(43) Pub. Date: Dec. 5, 2024

(54) COMPOUNDS TARGETING MUTANT OF P53

(71) Applicant: Jacobio Pharmaceuticals Co., Ltd., Beijing (CN)

(72) Inventors: Sujing LI, Beijing (CN); Amin LI, Beijing (CN); Qian ZHENG, Beijing (CN); Chaojie DANG, Beijing (CN); Xinrui FAN, Beijing (CN); Wei LONG, Beijing (CN); Yanping WANG, Beijing (CN)

(21) Appl. No.: 18/682,463

(22) PCT Filed: Aug. 9, 2022
(86) PCT No.: PCT/CN2022/111050

§ 371 Date: Feb. 9, 2024

(30) Foreign Application Priority Data
Aug. 10, 2021 (WO) PCT/CN2021/111797
Oct. 22, 2021 (WO) PCT/CN2021/125725
Nov. 23, 2021 (WO) PCT/CN2021/132409
Jan. 26, 2022 (WO) PCT/CN2022/073977
Jun. 9, 2022 (WO) PCT/CN2022/097840

(51) Int. Cl. C07F 9/6558; A61K 31/397; A61K 31/403; A61K 31/438; A61K 31/439; A61K 31/4535; A61K 31/454; A61K 31/4545; A61K 31/496; A61K 31/506; A61K 31/5377; A61K 31/541; A61K 31/55; A61K 31/67; A61K 31/675; C07D 333/58; C07D 403/12; C07D 409/12; C07D 409/14; C07D 413/14; C07D 417/14; C07D 471/08; C07D 487/04; C07D 491/107; C07D 495/04; C07F 9/6568; C07F 9/6584

(52) U.S. Cl. CPC C07F 9/65586; A61K 31/397; A61K 31/403; A61K 31/438; A61K 31/439; A61K 31/4535; A61K 31/454; A61K 31/4545; A61K 31/496; A61K 31/506; A61K 31/5377; A61K 31/541; A61K 31/55; A61K 31/67; A61K 31/675; C07D 333/58; C07D 403/12; C07D 409/12; C07D 409/14; C07D 413/14; C07D 417/14; C07D 471/08; C07D 487/04; C07D 491/107; C07D 495/04; C07F 9/65685; C07F 9/6584; C07F 9/65846

(57) ABSTRACT

Provided are compounds of formula (I) which can bind to mutant p53 and restore the ability of the p53 mutant to bind DNA and activate downstream effectors involved in tumor suppression. Also provided are pharmaceutical compositions comprising the compounds, methods of preparing the compounds and the use in the manufacture of medicaments for preventing or treating a disease or condition related to p53 mutants.

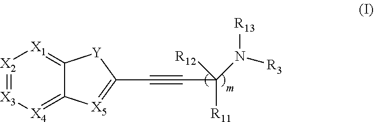

### CROSS REFERENCE TO THE RELATED APPLICATIONS

The present application claims the benefit of, and priority to PCT application PCT/CN2021/111797 filed on Aug. 10, 2021; PCT application PCT/CN2021/125725 filed on Oct. 22, 2021; PCT application PCT/CN2021/132409 filed on Nov. 23, 2021; PCT application PCT/CN2022/073977 filed on Jan. 26, 2022; and PCT application PCT/CN2022/097840 filed on Jun. 9, 2022; the contents of each of which are incorporated herein by reference in their entirety.

### FIELD OF THE INVENTION

The present invention relates to compounds targeting p53 mutants, pharmaceutical compositions comprising the compounds, methods of preparing the compounds and methods of using the compounds to prevent or treat a disease or condition related to p53 mutants.

### BACKGROUND OF THE INVENTION

The p53 protein, referred to as the “guardian of the human genome”, is a tetrameric transcription factor that prevents mutation to the genome by regulating the expression of a subgroup of target genes. Although biologically active as a homotetramer, each p53 monomer is comprised of 393 amino acids, and is divided into five key regulatory domains: the transactivation domain (TAD), proline-rich region (PR), the DNA binding domain (DBD), the oligomerization domain (OD), and the C-terminus.

Under normal conditions, the p53 protein has a “cancer suppressor” effect but p53 is unstable, with a half-life ranging from 5 to 30 minutes. Activation of p53 initiates pathways involved in apoptosis, DNA repair, cell cycle arrest, anti-angiogenesis, and senescence in order to avoid propagation of damaged cells. p53 activation occurs via a complicated regulatory network composed of three key steps: (1) p53 stabilization by phosphorylation, (2) DNA binding, and (3) target gene activation.

P53 is the most frequently mutated protein in human cancer. As examples, mutations are present in 96% of ovarian serous carcinomas, 87% of metastatic gastric cancers, 85% of small cell lung cancers, and 75% of pancreatic cancers, and are also associated with worsened prognosis and patient survival. Further, mutant p53 is a highly abundant and tumour-specific target as it is typically overexpressed in cancer, partly as a result of its inability to induce MDM2 gene expression in order to establish a negative feedback loop to control p53 expression. As a result of its overexpression, mutant p53 also possesses toxic GoF properties that can propagate and cause malfunctions to other important proteins and pathways that regulate the cell cycle. Considering the above factors, mutant p53 represents an important pharmacological target and the past two decades have seen considerable dedication to the development of small molecules that aim to restore wild-type function in mutant p53. In particular, targeting mutant p53 means to more selectively target the cancer cells, reducing the risk of side effects and toxicity towards healthy tissues. Among the small molecules developed, numerous mechanistic strategies have been developed including protein refolding via cysteine modification, protein stabilization, modulation of protein aggregation, and zinc chelation.

P53 is inactivated directly by mutation in 50% of human cancers (ranging from about 1% to 85% depending on the type of cancer), and almost all cancers exhibit malfunction along the p53 pathway. The frequency and aggressive nature of cancers exhibiting p53 malfunction has driven a widespread effort both in academia and the pharmaceutical industry to restore normal p53 expression and activity over the past decades. While this approach is faced with significant challenges including frequent off-target mechanisms of action, major technological advancements in gene sequencing capability and a shift towards personalized medicine holds significant promise for the development of small molecules capable of mutant-specific p53 reactivation.

Mutations in p53 located in the DNA binding domain of the protein or periphery of the DNA-binding surface result in aberrant protein folding required for DNA recognition and binding. Mutations in p53 can occur, for example, at amino acids Val143, His168, Arg175, Tyr220, Gly245, Arg248, Arg249, Phe270, Arg273, and Arg282. P53 mutations that can abrogate the activity of p53 include, for example, R175H, Y220C, G245S, R248Q, R248W, R273H, and R282W. These p53 mutations can either distort the structure of the DNA-binding site or thermodynamically destabilize the folded protein at body temperature. Wild-type function of p53 mutants can be recovered by binding of the p53 mutant to a compound that can shift the folding-unfolding equilibrium towards the folded state, thereby reducing the rate of unfolding and destabilization.

Due to the prevalence of p53 mutations in virtually every type of cancer, the reactivation of wild type p53 function in a cancerous cell can be an effective therapy. The p53 Y220C mutation is associated with many cancers, e.g., breast cancer, non-small cell lung cancer, colorectal cancer, pancreatic cancer, and ovarian cancer.

Notwithstanding PC14586 was reported to be a small molecule reactivator targeting p53 Y220C mutant developed by PMV Pharmaceuticals, Inc., there is still a critical need in the art for the development of new small molecule reactivators targeting p53 mutants (e.g., Y220C mutant) with high specificity and activity as well as low toxicity.

### SUMMARY OF THE INVENTION

In an aspect, it is an object of the present invention to provide a compound targeting mutant p53, preferably Y220C mutant.

In an aspect, it is an object of the present invention to provide a pharmaceutical composition comprising the above-mentioned compound targeting mutant p53.

In an aspect, it is an object of the present invention to provide a method for preventing or treating a disease or condition related to p53 mutant protein, which comprises administering to a subject a therapeutically effective amount of said compound or said pharmaceutical composition.

In an aspect, it is an object of the present invention to provide a method for preparation of a compound of the present invention.

In an aspect, it is an object of the present invention to provide an intermediate compound (such as formula (IN—I)) used for preparation of a compound of the present invention (such as formula (I)).

It is to be understood that each of the method of prevention or treatment embodiments herein can also be formulated as corresponding use type embodiments.

The present disclosure further provides the following aspect.

[1] A compound of formula (I), or a stereoisomer, tautomer, deuterated derivative, prodrug or pharmaceutically acceptable salt thereof:

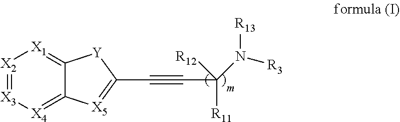

Y is selected from O, S, NR′, S═O, —S(═O)(═NR′)— or O═S═O;
one of X₁, X₂, X₃ and X₄ is selected from CR₂, and the others of X₁, X₂, X₃ and X₄ are each independently selected from N or CR₄;
X₅ is selected from N or CR₁;
R₁ is independently selected from hydrogen, deuterium, halogen, —C₁₋₆alkyl, —C₂₋₆alkenyl, —C₂₋₆alkynyl, —C₁₋₆haloalkyl, —CN, —OR′, —SR′, —C(O)R′, —C(O)N(R′)₂, —C(O)OR′, —OC(O)R′, —OC(O)N(R′)₂, —N(R′)₂, —NR′C(O)R′, —NR′C(O)OR′, —NR′C(O)N(R′)₂, —S(O)R′, —S(O)N(R′)₂, —NR′S(O)R′, —NR′S(O)N(R′)₂, —S(O)₂R′, —S(O)₂N(R′)₂, —NR′S(O)₂R′, —NR′S(O)₂N(R′)₂, —PO(R′)₂, 3-12 membered cycloalkyl, 3-12 membered cycloalkenyl, 3-12 membered heterocycloalkyl, 3-12 membered heterocycloalkenyl, 6-12 membered aryl or 5-12 membered heteroaryl; said —C₁₋₆alkyl, —C₂₋₆alkenyl, —C₂₋₆alkynyl, —C₁₋₆haloalkyl, 3-12 membered cycloalkyl, 3-12 membered cycloalkenyl, 3-12 membered heterocycloalkyl, 3-12 membered heterocycloalkenyl, 6-12 membered aryl, and 5-12 membered heteroaryl are each independently optionally substituted with one or more (such as 1, 2, 3, 4, 5 or 6) substituents selected from deuterium, halogen, —C₁₋₆alkyl, —C₂₋₆alkenyl, —C₂₋₆alkynyl, —C₁₋₆haloalkyl, —CN, oxo, —OR′, —SR′, —C(O)R′, —C(O)N(R′)₂, —C(O)OR′, —OC(O)R′, —OC(O)N(R′)₂, —N(R′)₂, —NR′C(O)R′, —NR′C(O)OR′, —NR′C(O)N(R′)₂, —S(O)R′, —S(O)N(R′)₂, —NR′S(O)R′, —NR′S(O)N(R′)₂, —S(O)₂R′, —S(O)₂N(R′)₂, —NR′S(O)₂R′, —NR′S(O)₂N(R′)₂, —PO(R′)₂, 3-12 membered cycloalkyl, 3-12 membered cycloalkenyl, 3-12 membered heterocycloalkyl, 3-12 membered heterocycloalkenyl, 6-12 membered aryl or 5-12 membered heteroaryl;
R₂ is —NR₅₁R₅₂, —OR₅₃ or —SR₅₄;
R₃ is selected from hydrogen, deuterium, —C₁₋₆alkyl, —C₂₋₆alkenyl, —C₂₋₆alkynyl, —C₁₋₆haloalkyl, —C(O)R′, —C(O)N(R′)₂, —C(O)OR′, —S(O)R′, —S(O)N(R′)₂, —S(O)₂R′, —S(O)₂N(R′)₂, —PO(R′)₂, 3-12 membered cycloalkyl, 3-12 membered cycloalkenyl, 3-12 membered heterocycloalkyl, 3-12 membered heterocycloalkenyl, 6-12 membered aryl, 5-12 membered heteroaryl; said —C₁₋₆alkyl, —C₂₋₆alkenyl, —C₂₋₆alkynyl, —C₁₋₆haloalkyl, 3-12 membered cycloalkyl, 3-12 membered cycloalkenyl, 3-12 membered heterocycloalkyl, 3-12 membered heterocycloalkenyl, 6-12 membered aryl, and 5-12 membered heteroaryl are each independently optionally substituted with one or more (such as 1, 2, 3, 4, 5 or 6) substituents of R₃ₐ;
each R₃ₐ is independently selected from deuterium, halogen, —C₁₋₆alkyl, —C₂₋₆alkenyl, —C₂₋₆alkynyl, —C₁₋₆haloalkyl, —CN, oxo, —OR′, —SR′, —C(O)R′, —C(O)N(R′)₂, —C(O)OR′, —OC(O)R′, —OC(O)N(R′)₂, —N(R′)₂, —NR′C(O)R′, —NR′C(O)OR′, —NR′C(O)N(R′)₂, —S(O)R′, —S(O)N(R′)₂, —NR′S(O)R′, —NR′S(O)N(R′)₂, —S(O)₂R′, —S(O)₂N(R′)₂, —S(═O)(═NR′)R′, —NR′S(O)₂R′, —NR′S(O)₂N(R′)₂, —PO(R′)₂, 3-12 membered cycloalkyl, 3-12 membered cycloalkenyl, 3-12 membered heterocycloalkyl, 3-12 membered heterocycloalkenyl, 6-12 membered aryl, 5-12 membered heteroaryl; said —C₁₋₆alkyl, —C₂₋₆alkenyl, —C₂₋₆alkynyl, —C₁₋₆haloalkyl, 3-12 membered cycloalkyl, 3-12 membered cycloalkenyl, 3-12 membered heterocycloalkyl, 3-12 membered heterocycloalkenyl, 6-12 membered aryl, and 5-12 membered heteroaryl are each independently optionally substituted with one or more substituents R_{3b},
each R_{3b} is independently selected from deuterium, halogen, —C₁₋₆alkyl, —C₂₋₆alkenyl, —C₂₋₆alkynyl, —C₁₋₆haloalkyl, —CN, oxo, —OR′, —SR′, —C(O)R′, —C(O)N(R′)₂, —C(O)OR′, —OC(O)R′, —OC(O)N(R′)₂, —N(R′)₂, —NR′C(O)R′, —NR′C(O)OR′, —NR′C(O)N(R′)₂, —S(O)R′, —S(O)N(R′)₂, —NR′S(O)R′, —NR′S(O)N(R′)₂, —S(O)₂R′, —S(O)₂N(R′)₂, —S(═O)(═NR′)R′, —NR′S(O)₂R′, —NR′S(O)₂N(R′)₂, —PO(R′)₂;
R₄ at each occurrence is independently selected from hydrogen, deuterium, halogen, —C₁₋₆alkyl, —C₂₋₆alkenyl, —C₂₋₆alkynyl, —C₁₋₆haloalkyl, —CN, —OR′, —SR′, —C(O)R′, —C(O)N(R′)₂, —C(O)OR′, —OC(O)R′, —OC(O)N(R′)₂, —N(R′)₂, —NR′C(O)R′, —NR′C(O)OR′, —NR′C(O)N(R′)₂, —S(O)R′, —S(O)N(R′)₂, —NR′S(O)R′, —NR′S(O)N(R′)₂, —S(O)₂R′, —S(O)₂N(R′)₂, —NR′S(O)₂R′, —NR′S(O)₂N(R′)₂, —PO(R′)₂, 3-12 membered cycloalkyl, 3-12 membered cycloalkenyl, 3-12 membered heterocycloalkyl, 3-12 membered heterocycloalkenyl, 6-12 membered aryl or 5-12 membered heteroaryl; said —C₁₋₆alkyl, —C₂₋₆alkenyl, —C₂₋₆alkynyl, —C₁₋₆haloalkyl, 3-12 membered cycloalkyl, 3-12 membered cycloalkenyl, 3-12 membered heterocycloalkyl, 3-12 membered heterocycloalkenyl, 6-12 membered aryl, and 5-12 membered heteroaryl are each independently optionally substituted with one or more (such as 1, 2, 3, 4, 5 or 6) substituents selected from deuterium, halogen, —C₁₋₆alkyl, —C₂₋₆alkenyl, —C₂₋₆alkynyl, —C₁₋₆haloalkyl, —CN, oxo, —OR′, —SR′, —C(O)R′, —C(O)N(R′)₂, —C(O)OR′, —OC(O)R′, —OC(O)N(R′)₂, —N(R′)₂, —NR′C(O)R′, —NR′C(O)OR′, —NR′C(O)N(R′)₂, —S(O)R′, —S(O)N(R′)₂, —NR′S(O)R′, —NR′S(O)N(R′)₂, —S(O)₂R′, —S(O)₂N(R′)₂, —S(═O)(═NR′)R′, —NR′S(O)₂R′, —NR′S(O)₂N(R′)₂, —PO(R′)₂, 3-12 membered cycloalkyl, 3-12 membered cycloalkenyl, 3-12 membered heterocycloalkyl, 3-12 membered heterocycloalkenyl, 6-12 membered aryl or 5-12 membered heteroaryl;
R₅₁, R₅₂, R₅₃ and R₅₄ are each independently selected from hydrogen, deuterium, —C₁₋₆alkyl, —C₂₋₆alkenyl, —C₂₋₆alkynyl, —C₁₋₆haloalkyl, —C(O)R′, —C(O)N(R′)₂, —C(O)OR′, —S(O)R′, —S(O)N(R′)₂, —S(O)₂R′, —S(O)₂N(R′)₂, —PO(R′)₂, 3-12 membered cycloalkyl, 3-12 membered cycloalkenyl, 3-12 membered heterocycloalkyl, 3-12 membered heterocycloalkenyl, 6-12 membered aryl, 5-12 membered heteroaryl; said —C₁₋₆alkyl, —C₂₋₆alkenyl, —C₂₋₆alkynyl, —C₁₋₆haloalkyl, 3-12 membered cycloalkyl, 3-12 membered cycloalkenyl, 3-12 membered heterocycloalkyl, 3-12 membered heterocycloalkenyl, 6-12 membered aryl, and 5-12 membered heteroaryl are each independently optionally substituted with one or more (such as 1, 2, 3, 4, 5 or 6) substituents selected from deuterium, halogen, —C₁₋₆alkyl, —C₂₋₆alkenyl, —C₂₋₆alkynyl, —C₁₋₆haloalkyl, —CN, oxo, ═NR′, —C₁₋₆alkyl-CH(R′)₂, —OR′, —SR′, —C(O)R′, —C(O)N(R′)₂, —C(O)OR′, —OC(O)R′, —OC(O)N(R′)₂, —N(R′)₂, —NR′C(O)R′, —NR′C(O)OR′, —NR′C(O)N(R′)₂, —S(O)R′, —S(O)N(R′)₂, —NR′S(O)R′, —NR′S(O)N(R′)₂, —S(O)₂R′, —S(O)₂N(R′)₂, —S(═O)(═NR′)R′, —NR′S(O)₂R′, —NR′S(O)₂N(R′)₂, —PO(R′)₂, 3-12 membered cycloalkyl, 3-12 membered cycloalkenyl, 3-12 membered heterocycloalkyl, 3-12 membered heterocycloalkenyl, 6-12 membered aryl, or 5-12 membered heteroaryl;
R₁₁ and R₁₂ are independently selected from hydrogen, deuterium, —OH, halogen, —CN, oxo, —C₁₋₆alkyl, —C₁₋₆ haloalkyl, —C₁₋₆alkoxy, —NH₂, —NHC₁₋₆alkyl, —N(C₁₋₆alkyl)₂ or 3-6 membered cycloalkyl; wherein said —C₁₋₆alkyl, —C₁₋₆alkoxy and 3-6 membered cycloalkyl are each independently optionally substituted with one or more (such as 1, 2, 3, 4, 5 or 6) substituents selected from deuterium, —OH, halogen, —CN, oxo, —C₁₋₆alkoxy, —NH—C₁₋₆alkyl, —N(C₁₋₄ alkyl)₂, or 3-6 membered cycloalkyl;
R₁₃ is selected from hydrogen, deuterium, —C₁₋₆alkyl or 3-6 membered cycloalkyl; wherein said —C₁₋₆alkyl and 3-6 membered cycloalkyl are each independently optionally substituted with one or more (such as 1, 2, 3, 4, 5 or 6) substituents selected from deuterium, —OH, halogen, —CN, oxo, —C₁₋₆alkoxy, —NH₂, —NHC₁₋₆alkyl, or —N(C₁₋₄ alkyl)₂;
each R′ at each occurrence is independently selected from hydrogen, deuterium, halogen, —OH, —CN, oxo, —NH₂, —NHC₁₋₆ alkyl, —N(C₁₋₆ alkyl)₂, —C₁₋₆alkyl, —C₁₋₆alkylOC₁₋₆alkyl, —C₁₋₆alkyl-NHC₁₋₆alkyl, —C₁₋₆alkyl-N(C₁₋₆alkyl)₂, —C₁₋₆haloalkyl, —OC₁₋₆alkyl, —C₃₋₁₄cycloalkyl, —C₃₋₁₄heterocycloalkyl, —C₂₋₆alkenyl, —C₂₋₆alkynyl, 6-12 membered aryl, or 5-12 membered heteroaryl; wherein said —C₁₋₆alkyl, —OC₁₋₆alkyl, —C₃₋₁₄cycloalkyl, —C₃₋₁₄heterocycloalkyl, —C₂₋₆alkenyl, —C₂₋₆alkynyl, 6-12 membered aryl, or 5-12 membered heteroaryl is independently optionally substituted with 1, 2, 3, 4, 5 or 6 substituents selected from deuterium, halogen, —CN, —C₁₋₃alkyl, oxo, —OH, —OC₁₋₃alkyl, —NH₂, —NHC₁₋₃alkyl, —N(C₁₋₃alkyl)₂, —C(═O)NH₂, —C(═O)NH(C₁₋₃alkyl), —C(═O)N(C₁₋₃alkyl)₂, —S(═O)₂NH₂, —S(═O)₂NH(C₁₋₃alkyl), —S(═O)₂C₁₋₃alkyl, —S(═O)₂N(C₁₋₃alkyl)₂, —S(═O)(═NH)C₁₋₃alkyl, —S(═O)(═NC₁₋₃alkyl)C₁₋₃alkyl, or 3-6 membered cycloalkyl;
said heterocycloalkyl, heterocycloalkenyl, and heteroaryl each independently contains 1, 2 or 3 heteroatoms selected from N, O, P or S;
m is selected from 1, 2, 3, 4, 5 or 6.

[2] The compound according to [1], wherein,
the formula (I) is:

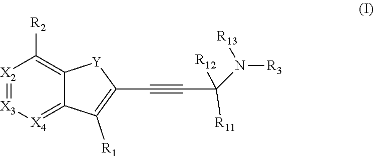

X₂, X₃, and X₄ in the formula (I-1) are each independently selected from N or CR₄;
Y is selected from O, S, S═O, or O═S═O.

[3] The compound according to [1] or [2], wherein, the formula (I) is:

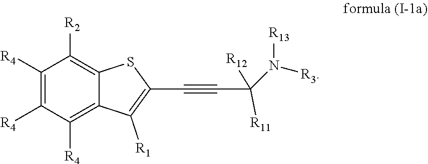

[4] The compound according to [1] or [2], wherein, the formula (I) is:

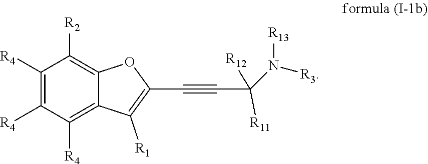

[5] The compound according to [1] or [2], wherein, the formula (I) is:

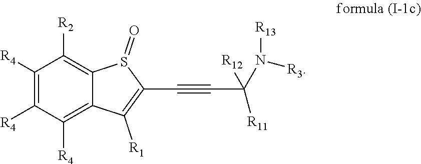

[6] The compound according to [1] or [2], wherein, the formula (I) is:

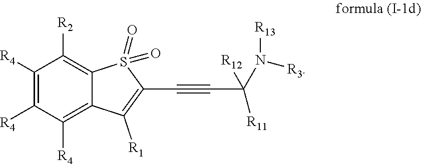

[7] The compound according to [1] or [2], wherein, the formula (I) is:

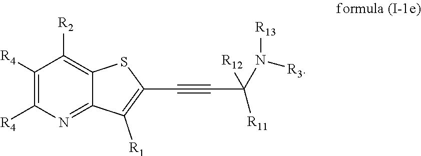

[8] The compound according to [1] or [2], wherein, the formula (I) is:

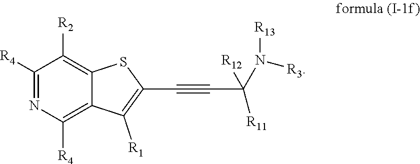

[9] The compound according to [1] or [1], wherein, the formula (I) is:

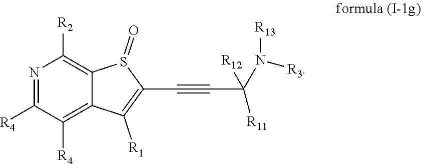

[10] The compound according to any one of [1] to [3], wherein, the formula (I) is selected from:

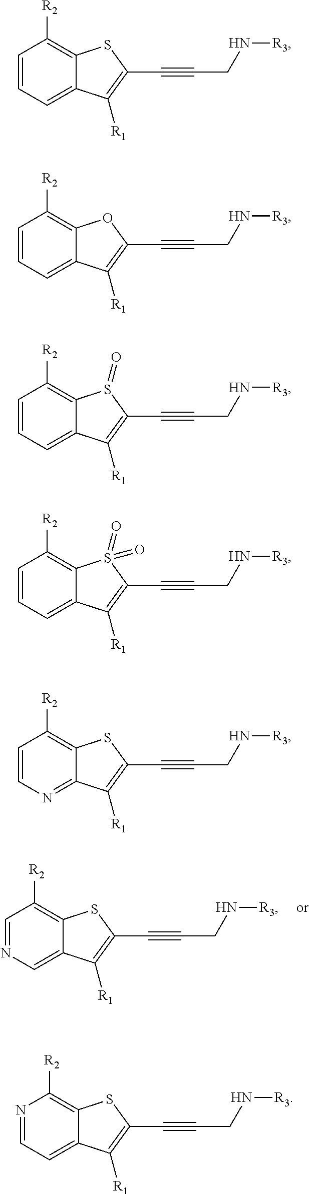

[11] The compound according to any one of [1] to [10], wherein, R₁ is independently selected from halogen, —C₁₋₆alkyl, —C₂₋₆alkenyl, —C₂₋₆alkynyl, —C₁₋₆haloalkyl, —CN, 3-12 membered cycloalkyl, 3-12 membered cycloalkenyl, 3-12 membered heterocycloalkyl, 3-12 membered heterocycloalkenyl, 6-10 membered aryl, or 5-12 membered heteroaryl, said —C₁₋₆alkyl, —C₂₋₆alkenyl, —C₂₋₆alkynyl, —C₁₋₆haloalkyl, 3-12 membered cycloalkyl, 3-12 membered cycloalkenyl, 3-12 membered heterocycloalkyl, 3-12 membered heterocycloalkenyl, 6-10 membered aryl, and 5-12 membered heteroaryl are independently optionally substituted with one or more (such as 1, 2, 3, 4, 5 or 6) substituents selected from halogen, —C₁₋₆alkyl, —C₂₋₆alkenyl, —C₂₋₆alkynyl, —C₁₋₆haloalkyl, —CN, oxo, 3-12 membered cycloalkyl, 3-12 membered cycloalkenyl, 3-12 membered heterocycloalkyl, 3-12 membered heterocycloalkenyl, 6-10 membered aryl, or 5-12 membered heteroaryl, said heterocycloalkyl, heterocycloalkenyl, and heteroaryl each independently contains 1 or 2 heteroatoms selected from N or S.

[12] The compound according to any one of [1] to [11], wherein, R₁ is independently selected from —F, —Cl, —C₁₋₃alkyl, —C₂₋₄alkenyl, —C₂₋₄alkynyl, —C₁₋₃haloalkyl, —CN, 3-6 membered cycloalkyl, 3-6 membered heterocycloalkyl, 6-10 membered aryl, 5-12 membered heteroaryl, said —C₁₋₃alkyl, —C₂₋₄alkenyl, —C₂₋₄alkynyl, —C₁₋₃haloalkyl, 3-6 membered cycloalkyl, 3-6 membered heterocycloalkyl, 6-10 membered aryl, and 5-12 membered heteroaryl are independently optionally substituted with one or more (such as 1, 2, 3, 4, 5 or 6) substituents selected from —F, —Cl, —C₁₋₃alkyl, —C₂₋₄alkenyl, —C₂₋₄alkynyl, —C₁₋₃haloalkyl, —CN, oxo, 3-6 membered cycloalkyl, 3-6 membered heterocycloalkyl, 6-10 membered aryl, or 5-12 membered heteroaryl, said heterocycloalkyl, heterocycloalkenyl, and heteroaryl each independently contains 1 or 2 heteroatoms selected from N or S.

[13] The compound according to any one of [1] to [12], wherein, R₁ is independently selected from —F, —Cl, —C₁₋₃alkyl, —C₁₋₃haloalkyl, 3-6 membered cycloalkyl, 3-6 membered heterocycloalkyl, phenyl, or 5-6 membered heteroaryl, said —C₁₋₃alkyl, —C₁₋₃haloalkyl, 3-6 membered cycloalkyl, 3-6 membered heterocycloalkyl, phenyl, or 5-6 membered heteroaryl are independently optionally substituted with one or more (such as 1, 2, 3, 4, 5 or 6) substituents selected from —F, —Cl, —C₁₋₃alkyl, —C₁₋₃haloalkyl, 3-6 membered cycloalkyl, 3-6 membered heterocycloalkyl, phenyl, or 5-6 membered heteroaryl, said heterocycloalkyl, heterocycloalkenyl, and heteroaryl each independently contains 1 or 2 heteroatoms selected from N or S.

[14] The compound according to any one of [1] to [12], wherein R₁ is independently selected from —F, —Cl, —C₁₋₃alkyl, —C₁₋₃haloalkyl, 3-6 membered cycloalkyl, 5 membered heteroaryl containing 1 or 2 heteroatoms selected from N, O or S, or 6 membered heteroaryl containing 1 or 2 heteroatoms selected from N; said —C₁₋₃alkyl, —C₁₋₃haloalkyl, 3-6 membered cycloalkyl, 5 membered heteroaryl and 6 membered heteroaryl are each independently optionally substituted with 1, 2 or 3 substituents selected from —F, —C₁₋₃alkyl, —OC₁₋₃alkyl, —NH₂, —NHC₁₋₃alkyl, —N(C₁₋₃alkyl)₂, —CN or 3-6 membered cycloalkyl.

[15] The compound according to any one of [1] to [14], wherein R₁ is independently selected from —C₁₋₃alkyl; —C₁₋₃haloalkyl; 5 membered heteroaryl containing 1 or 2 heteroatoms selected from N, O or S; or —C₁₋₃alkyl substituted with 1, 2 or 3 substituents selected from —F, —OC₁₋₃alkyl, —NH₂, —NHC₁₋₃alkyl, —N(C₁₋₃alkyl)₂, —CN or 3-6 membered cycloalkyl.

[16] The compound according to any one of [1]-[15], wherein, R₁ is independently selected from

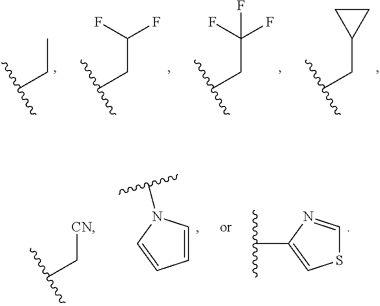

[17] The compound according to any one of [1] to [16], wherein, R₂ is —NR₅₁R₅₂.

[18] The compound according to any one of [1] to [17], wherein, R₂ is —NHR₅₁.

[19] The compound according to any one of [1] to[18], wherein, R₅₁ is selected from —C₁₋₆alkyl, —C₂₋₆alkenyl, —C₂₋₆alkynyl, —C₁₋₆haloalkyl, —C(O)R′, —C(O)N(R′)₂, —C(O)OR′, 3-6 membered cycloalkyl, 3-6 membered heterocycloalkyl, phenyl, or 5-6 membered heteroaryl; said —C₁₋₆alkyl, —C₂₋₆alkenyl, —C₂₋₆alkynyl, —C₁₋₆haloalkyl, 3-6 membered cycloalkyl, 3-6 membered heterocycloalkyl, phenyl, 5-6 membered heteroaryl are each independently optionally substituted with one or more (such as 1, 2, 3, 4, 5 or 6) substituents selected from halogen, —C₁₋₆alkyl, —C₂₋₆alkenyl, —C₂₋₆alkynyl, —C₁₋₆haloalkyl, —CN, oxo, ═NR′, —C₁₋₆alkyl-CH(R′)₂, —OR′, —SR′, —C(O)R′, —C(O)N(R′)₂, —C(O)OR′, —OC(O)R′, —OC(O)N(R′)₂, —N(R′)₂, —NR′C(O)R′, —NR′C(O)OR′, —NR′C(O)N(R′)₂, 3-6 membered cycloalkyl, 3-6 membered heterocycloalkyl, phenyl, or 5-6 membered heteroaryl, said heterocycloalkyl, heterocycloalkenyl, and heteroaryl each independently contains 1 heteroatom selected from N, O, or S.

[20] The compound according to any one of [1] to [19], wherein, R₅₁ is selected from —C₁₋₃alkyl, cyclopentyl, cyclohexyl, 5 membered heterocyclyl containing 1 heteroatom selected from N, O, S, S(═O), S(═O)(═NH) or S(═O)₂ or 6 membered heterocyclyl containing 1 heteroatom selected from N, O, S, S(═O), S(═O)(═NH) or S(═O)₂; said —C₁₋₃alkyl, cyclopentyl, cyclohexyl, 5 membered heterocyclyl and 6 membered heterocyclyl are each independently optionally substituted with 1, 2 or 3 substituents selected from —F, —C₁₋₃alkyl, —OC₁₋₃alkyl, —NH₂, —NHC₁₋₃alkyl, oxo, ═NH, —N(C₁₋₃alkyl)₂, —CN or 3-6 membered cycloalkyl.

[21] The compound according to any one of [1] to [20], wherein, R₅₁ is selected from —C₁₋₆alkyl, 5 membered cycloalkyl, 5 membered heterocycloalkyl, 6 membered cycloalkyl, or 6 membered heterocycloalkyl; said —C₁₋₆alkyl, cycloalkyl, and heterocycloalkyl are independently optionally substituted with one or more (such as 1, 2, 3, 4, 5 or 6) substituents selected from —F, —Cl, —CH₃, —CD₃, —CH₂CH₃, —CH₂CH₂CH₃, —CH(CH₃)₂, —CH(OH)CH₂(OH), —CH(OCH₃)CH₂(OH), —CH(OH)CH₂(OCH₃), —CH₂CH(OH)(OCH₃), —CH₂CH(OH)(OCH₂CH₃), —CH₂CH(OCH₃)₂, —OH, —O—CH₃, —O—CH₂CH₃, —O—CH₂CH₂CH₃, —O—CH(CH₃)₂, —C(O)—(CH₂)—NH₂, —C(O)—(CH₂)—NH(CH₃), —C(O)—(CH₂)—NH(CH₂CH₃), —C(O)—(CH₂)—N(CH₃)₂, —C(O)—(CH₂CH₂)—NH₂, —C(O)—(CH₂CH₂)—NH(CH₃), —C(O)—(CH₂CH₂)—NH(CH₂CH₃), —C(O)—(CH₂CH₂)—N(CH₃)₂, —NH₂, —NH(CH₃), —NH(CH₂CH₃), —N(CH₃)₂, —NH(CH₂CH₂CH₃), —NH(CH(CH₃)₂), or —N(CH₃)(CH₂CH₃), said heterocycloalkyl, heterocycloalkenyl, and heteroaryl each independently contains 1 heteroatom selected from N, O, or S.

[22] The compound according to any one of [1] to [21], wherein, R₅₁ is selected from

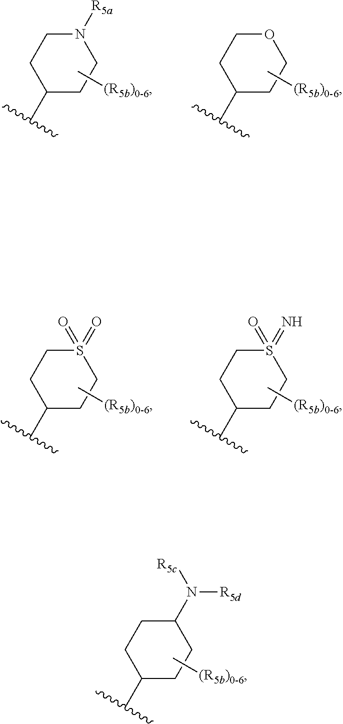

or —C₁₋₆alkyl, said —C₁₋₆alkyl is optionally substituted with 1, 2, 3, 4, 5, or 6 R₅ₑ;
R₅ₐ, R_{5c} and R_{5d} are each independently selected from hydrogen; —C₁₋₆alkyl; or —C₁₋₆alkyl substituted with 1, 2, 3, 4, 5 or 6 substituents selected from —F, —C₁₋₃alkyl, oxo, —OC₁₋₃alkyl, —NH₂, —NHC₁₋₃alkyl, —N(C₁₋₃alkyl)₂, —CN or 3-6 membered cycloalkyl;
R_{5b} and R₅ₑ are each independently selected from —F, —C₁₋₆alkyl, oxo, —OC₁₋₆alkyl, —NH₂, —NHC₁₋₆alkyl, —N(C₁₋₆alkyl)₂, —CN or 3-6 membered cycloalkyl, wherein, said —C₁₋₆alkyl is optionally substituted with 1, 2, 3 or 4 substituents selected from —F, —C₁₋₃alkyl, oxo, —OC₁₋₃alkyl, —NH₂, —NHC₁₋₃alkyl, —N(C₁₋₃alkyl)₂, —CN or 3-6 membered cycloalkyl.

[23] The compound according to any one of [1] to [22], wherein, R₅₁ is selected from

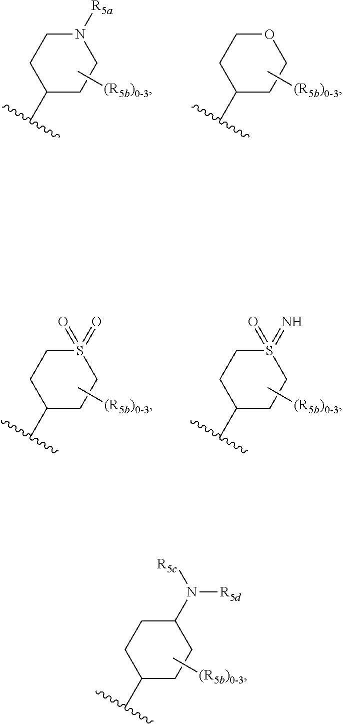

or —C₁₋₃alkyl-N(C₁₋₃alkyl)₂;
R₅ₐ is independently selected from —C₁₋₃alkyl; or —C₁₋₃alkyl substituted with 1, 2, 3, 4, 5 or 6 substituents selected from —F, —C₁₋₃alkyl, oxo, —OC₁₋₃alkyl, —NH₂, —NHC₁₋₃alkyl, —N(C₁₋₃alkyl)₂, —CN or 3-6 membered cycloalkyl;
R_{5b} is independently selected from —F, —C₁₋₃alkyl, oxo, —OC₁₋₃alkyl, —NH₂, —NHC₁₋₃alkyl, —N(C₁₋₃alkyl)₂ or —CN;
R_{5c} and R_{5d} are each independently selected from —C₁₋₃alkyl.

[24] The compound according to any one of [1] to [22], wherein, R₅₁ is selected from

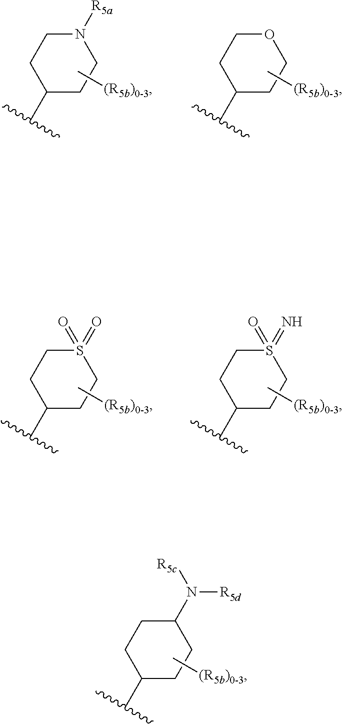

or —CH₂CH₂—N(CH₃)₂;
R₅ₐ is independently selected from methyl, —CH₂CH(OH)OCH₃ or —C(═O)CH₂N(CH₃)₂;
R_{5b} is independently selected from —F;
R_{5c} and R_{5d} are each independently selected from methyl.

[25] The compound according to any one of [1] to [24], wherein R₅₁ is selected from

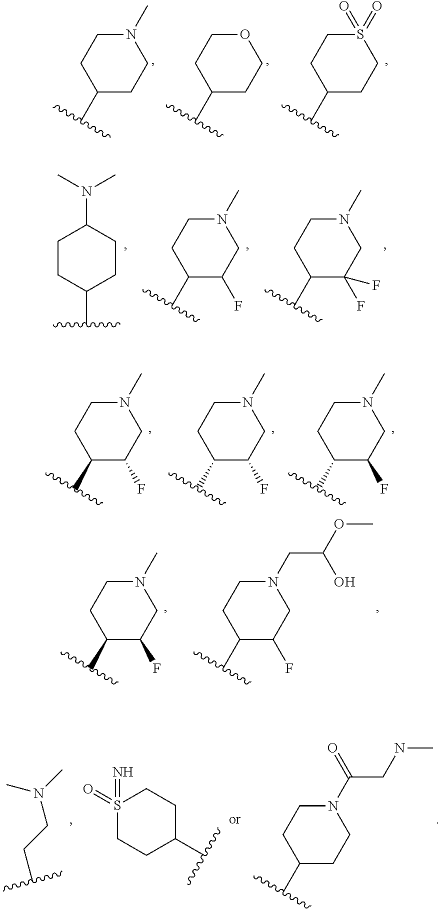

[26] The compound according to any one of [1]-[25], wherein R₃ is independently selected from hydrogen, deuterium, halogen, —C₁₋₆alkyl, —C₂₋₆alkenyl, —C₂₋₆alkynyl, —C₁₋₆haloalkyl, —CN, —NO₂, —OR′, —SR′, —C(O)R′, —C(O)N(R′)₂, —C(O)OR′, —OC(O)R′, —N(R′)₂, —NR′C(O)R′, —S(O)R′, —NR′S(O)R′, —S(O)N(R′)₂, —S(O)₂R′, —NR′S(O)₂R′, —S(O)₂N(R′)₂, 3-12 membered cycloalkyl, 3-12 membered cycloalkenyl, 3-12 membered heterocycloalkyl, 3-12 membered heterocycloalkenyl, 6-10 membered aryl, or 5-12 membered heteroaryl; said —C₁₋₆alkyl, —C₂₋₆alkenyl, —C₂₋₆alkynyl, —C₁₋₆haloalkyl, 3-12 membered cycloalkyl, 3-12 membered cycloalkenyl, 3-12 membered heterocycloalkyl, 3-12 membered heterocycloalkenyl, 6-10 membered aryl, or 5-12 membered heteroaryl at each occurrence is independently optionally substituted with one or more substituents selected from halogen, NH₂, NH—C₁₋₆alkyl, —C₁₋₆alkyl, —C₂₋₆alkenyl, —C₂₋₆alkynyl, —C₁₋₆haloalkyl, —CN, —NO₂, —OR′, —SR′, —C(O)R′, oxo, —C(O)N(R′)₂, —C(O)OR′, —OC(O)R′, —N(R′)₂, —NR′C(O)R′, —S(O)R′, —NR′S(O)R′, —S(O)N(R′)₂, —S(O)₂R′, —NR′S(O)₂R′, —S(O)₂N(R′)₂, —S(═O)(═NR′)R′, 3-12 membered cycloalkyl, 3-12 membered cycloalkenyl, 3-12 membered heterocycloalkyl, 3-12 membered heterocycloalkenyl, 6-10 membered aryl, or 5-12 membered heteroaryl, said heterocycloalkyl, heterocycloalkenyl, and heteroaryl each independently contains 1 heteroatom selected from N, O, or S.

[27] The compound according to any one of [1] to [25], wherein R₃ is independently selected from phenyl, or 5-6 membered heteroaryl containing 1 or 2 heteroatoms selected from N, O or S; said phenyl and 5-6 membered heteroaryl are independently optionally substituted with 1, 2, 3 substituents selected from R_{3c};
R₃ₑ is selected from —F; —C₁₋₃alkyl; —OC₁₋₃alkyl; —OC₃₋₆cycloalkyl; —NH₂; —NHC₁₋₃alkyl; —N(C₁₋₃alkyl)₂; —C(═O)NH₂, —C(═O)NH(C₁₋₃alkyl); —C(═O)N(C₁₋₃alkyl)₂; —CN; —S(═O)₂NH₂; —S(═O)₂NH(C₁₋₃alkyl); —S(═O)₂NHC(═O)C₁₋₃alkyl; —S(═O)₂N(C₁₋₃alkyl)₂; —S(═O)₂C₁₋₃alkyl; —S(═O)(═NH)C₁₋₃alkyl; —S(═O)(═NH)C₃₋₆cycloalkyl; —S(═O)(═NC₃₋₆cycloalkyl)C₁₋₃alkyl; —S(═O)(═NC₂₋₆heterocycloalkyl)C₁₋₃alkyl; —S(═O)(═NC₁₋₃alkyl)C₁₋₃alkyl; —S(═O)(═NCN)C₁₋₃alkyl; or 3-6 membered cycloalkyl; wherein said C₁₋₃alkyl, C₃₋₆cycloalkyl, C₂₋₆heterocycloalkyl or OC₁₋₃alkyl at each occurrence is independently optionally substituted with 1, 2, 3, 4, 5 or 6 substituents selected from deuterium, —F, —C₁₋₃alkyl, oxo, —OH, —OC₁₋₃alkyl, —NH₂, —NHC₁₋₃alkyl, —N(C₁₋₃alkyl)₂, —C(═O)NH₂, —C(═O)NH(C₁₋₃alkyl), —C(═O)N(C₁₋₃alkyl)₂, —CN, —S(═O)₂NH₂, —S(═O)₂NH(C₁₋₃alkyl), —S(═O)₂C₁₋₃alkyl, —S(═O)₂N(C₁₋₃alkyl)₂, —S(═O)(═NH)C₁₋₃alkyl, —S(═O)(═NC₁₋₃alkyl)C₁₋₃alkyl, or 3-6 membered cycloalkyl.

[28] The compound according to any one of [1] to [27], wherein R₃ is independently selected from phenyl or pyridinyl.

[29] The compound according to any one of [1] to [28], wherein R₃ is independently selected from phenyl, 5 membered heteroaryl, or 6 membered heteroaryl; said phenyl and heteroaryl at each occurrence are independently optionally substituted with one or more (such as 1, 2, 3, 4, 5 or 6) substituents selected from —F, —Cl, —CH₃, —CD₃, —CH₂CH₃, —CH₂CH₂CH₃, —CH(CH₃)₂, —CH₂F, —CHF₂, —CF₃, —CH₂CH₂F, —CH₂CHF₂, —CH₂CF₃, —CHFCH₃, —CF₂CH₃, —CHFCH₂F, —CH₂CHFCH₃, —CH₂CF₂CH₃, —CH₂CH₂CF₃, —C(CH₃)₂F, —CN, —OH, —O—CH₃, —O—CH₂CH₃, —O—CH₂CH₂CH₃, —O—CH(CH₃)₂, —SH, —S—CH₃, —S—CH₂CH₃, —S—CH₂CH₂CH₃, —S—CH(CH₃)₂, —CHO, —C(O)—CH₃, —C(O)—CH₂CH₃, —C(O)—CH₂CH₂CH₃, —C(O)—CH(CH₃)₂, —C(O)NH₂, —C(O)NH(CH₃), —C(O)NH(CH₂CH₃), —C(O)N(CH₃)₂, —C(O)NH(CH₂CH₂CH₃), —C(O)NH(CH(CH₃)₂), —C(O)N(CH₃)(CH₂CH₃), —NH₂, —NH(CH₃), —NH(CH₂CH₃), —N(CH₃)₂, —NH(CH₂CH₂CH₃), —NH(CH(CH₃)₂), —N(CH₃)(CH₂CH₃), —NHC(O)(CH₃), —NHC(O)(CH₂CH₃), —NHC(O)(CH₃)₂, —NHC(O)(CH₂CH₂CH₃), —NHC(O)(CH(CH₃)₂), —NHC(O)(CH₃)(CH₂CH₃), —S(O)₂H, —S(O)₂(CH₃), —S(O)₂(CH₂CH₃), —S(O)₂(CH₃)₂, —S(O)₂(CH₂CH₂CH₃), —S(O)₂(CH(CH₃)₂), —S(O)₂(CH₃)(CH₂CH₃), —S(O)₂NH₂, —S(O)₂NH(CH₃), —S(O)₂NH(CH₂CH₃), —S(O)₂N(CH₃)₂, —S(O)₂NH(CH₂CH₂CH₃), —S(O)₂NH(CH(CH₃)₂), —S(O)₂N(CH₃)(CH₂CH₃), —S(═O)(═NH)CH₃, or —S(═O)(═NCH₃)CH₃, said heterocycloalkyl, heterocycloalkenyl, and heteroaryl each independently contains 1 heteroatom selected from N, O, or S.

[30] The compound according to any one of [1] to [29], wherein R₃ is independently selected from phenyl, 5 membered heteroaryl, or 6 membered heteroaryl; said phenyl and heteroaryl at each occurrence is independently optionally substituted with one or more (such as 1, 2, 3, 4, 5 or 6) substituents selected from —F, —CH₃, —CD₃, —CH₂CH₃, —CH₂CH₂CH₃, —CH(CH₃)₂, —CH₂F, —CF₃, —CH₂CH₂F, —CHFCH₃, —CF₂CH₃, —C(CH₃)₂F, —OH, —O—CH₃, —O—CH₂CH₃, —O—CH₂CH₂CH₃, —O—CH(CH₃)₂, —C(O)NH₂, —C(O)NH(CH₃), —C(O)NH(CH₂CH₃), —C(O)N(CH₃)₂, —C(O)NH(CH₂CH₂CH₃), —C(O)NH(CH(CH₃)₂), —C(O)N(CH₃)(CH₂CH₃), —NH₂, —NH(CH₃), —NH(CH₂CH₃), —N(CH₃)₂, —NH(CH₂CH₂CH₃), —NH(CH(CH₃)₂), —N(CH₃)(CH₂CH₃), —S(O)₂H, —S(O)₂(CH₃), —S(O)₂(CH₂CH₃), —S(O)₂(CH₃)₂, —S(O)₂(CH₂CH₂CH₃), —S(O)₂(CH(CH₃)₂), —S(O)₂(CH₃)(CH₂CH₃), —S(O)₂NH₂, —S(O)₂NH(CH₃), —S(O)₂NH(CH₂CH₃), —S(O)₂N(CH₃)₂, —S(O)₂NH(CH₂CH₂CH₃), —S(O)₂NH(CH(CH₃)₂), —S(O)₂N(CH₃)(CH₂CH₃), —S(═O)(═NH)CH₃, or —S(═O)(═NCH₃)CH₃, said heterocycloalkyl, heterocycloalkenyl, and heteroaryl each independently contains 1 heteroatom selected from N, O, or S.

[31] The compound according to any one of [1] to [30], wherein R₃ is independently selected from

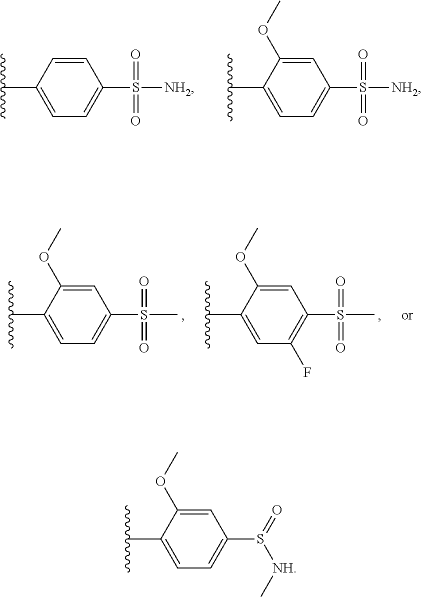

[32] The compound according to any one of [1] to [31], wherein the formula (I) is:

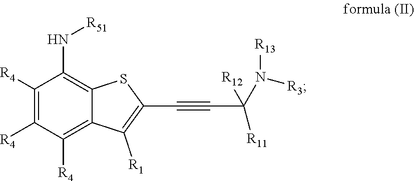

wherein,
R₅₁ is selected from —C₁₋₃alkyl, cyclopentyl, cyclohexyl, 5 membered heterocyclyl containing 1 heteroatom selected from N, O, S, S(═O), S(═O)(═NH) or S(═O)₂ or 6 membered heterocyclyl containing 1 heteroatom selected from N, O, S, S(═O), S(═O)(═NH) or S(═O)₂; said —C₁₋₃alkyl, cyclopentyl, cyclohexyl, 5 membered heterocyclyl and 6 membered heterocyclyl are each independently optionally substituted with 1, 2 or 3 substituents selected from —F, —C₁₋₃alkyl, —OC₁₋₃alkyl, —NH₂, —NHC₁₋₃alkyl, oxo, —N(C₁₋₃alkyl)₂, —CN or 3-6 membered cycloalkyl;
R₁ is independently selected from —F, —Cl, —C₁₋₃alkyl, —C₂₋₄alkenyl, —C₂₋₄alkynyl, —C₁₋₃haloalkyl, —CN, 3-6 membered cycloalkyl, 3-6 membered heterocycloalkyl, 6-10 membered aryl, 5-12 membered heteroaryl, said —C₁₋₃alkyl, —C₂₋₄alkenyl, —C₂₋₄alkynyl, —C₁₋₃haloalkyl, 3-6 membered cycloalkyl, 3-6 membered heterocycloalkyl, 6-10 membered aryl, and 5-12 membered heteroaryl are independently optionally substituted with one or more (such as 1, 2, 3, 4, 5 or 6) substituents selected from —F, —Cl, —C₁₋₃alkyl, —C₂₋₄alkenyl, —C₂₋₄alkynyl, —C₁₋₃haloalkyl, —CN, oxo, 3-6 membered cycloalkyl, 3-6 membered heterocycloalkyl, 6-10 membered aryl, or 5-12 membered heteroaryl, said heterocycloalkyl, heterocycloalkenyl, and heteroaryl each independently contains 1 or 2 heteroatoms selected from N or S;
each of R₄ is independently selected from hydrogen, deuterium, —F, —Cl, —C₁₋₃alkyl, —OC₁₋₃alkyl, —NH₂, —NHC₁₋₃alkyl, —N(C₁₋₃alkyl)₂, —CN or 3-6 membered cycloalkyl;
R₁₁ and R₁₂ are independently selected from hydrogen, deuterium, —F, —C₁₋₃alkyl, oxo, —OC₁₋₃alkyl, —NH₂, —NHC₁₋₃alkyl, —N(C₁₋₃alkyl)₂, —CN or 3-6 membered cycloalkyl, wherein, said —C₁₋₃alkyl is optionally substituted with 1, 2, 3 or 4 substituents selected from —F, —C₁₋₃alkyl, oxo, —OC₁₋₃alkyl, —NH₂, —NHC₁₋₃alkyl, —N(C₁₋₃alkyl)₂, —CN or 3-6 membered cycloalkyl;
R₁₃ is selected from hydrogen; —C₁₋₃alkyl; or —C₁₋₃alkyl substituted with 1, 2, 3, 4, 5 or 6 substituents selected from —F, —C₁₋₃alkyl, oxo, —OC₁₋₃alkyl, —NH₂, —NHC₁₋₃alkyl, —N(C₁₋₃alkyl)₂, —CN or 3-6 membered cycloalkyl;
R₃ is independently selected from phenyl, or 5-6 membered heteroaryl containing 1 or 2 heteroatoms selected from N, O or S; said phenyl and 5-6 membered heteroaryl are independently optionally substituted with 1, 2, or 3 substituents selected from R_{3c};
R₃ₑ is selected from —F; —C₁₋₃alkyl; —OC₁₋₃alkyl; —NH₂; —NHC₁₋₃alkyl; —N(C₁₋₃alkyl)₂; —C(═O)NH₂, —C(═O)NH(C₁₋₃alkyl); —C(═O)N(C₁₋₃alkyl)₂; —CN; —S(═O)₂NH₂; —S(═O)₂NH(C₁₋₃alkyl); —S(═O)₂N(C₁₋₃alkyl)₂; 3-6 membered cycloalkyl; or —C₁₋₃alkyl substituted with 1, 2, 3, 4, 5 or 6 substituents selected from —F, —C₁₋₃alkyl, oxo, —OC₁₋₃alkyl, —NH₂, —NHC₁₋₃alkyl, —N(C₁₋₃alkyl)₂, —C(═O)NH₂, —C(═O)NH(C₁₋₃alkyl), —C(═O)N(C₁₋₃alkyl)₂, —CN, —S(═O)₂NH₂, —S(═O)₂NH(C₁₋₃alkyl), —S(═O)₂N(C₁₋₃alkyl)₂, —S(═O)(═NH)C₁₋₃alkyl, —S(═O)(═NC₁₋₃alkyl)C₁₋₃alkyl, or 3-6 membered cycloalkyl.

[33] The compound according to [32], wherein,
R₅₁ is selected from

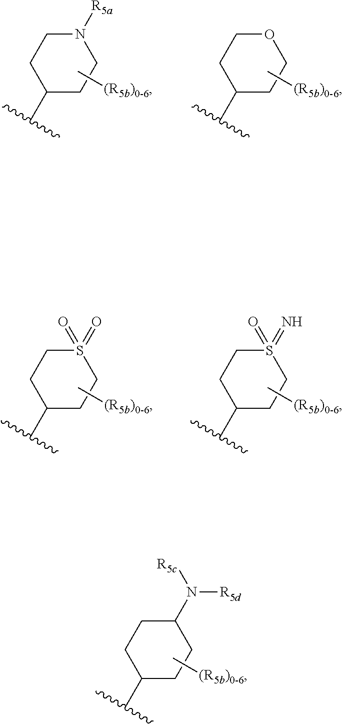

or —C₁₋₆alkyl, said —C₁₋₆alkyl is optionally substituted with 1, 2, 3, 4, 5, or 6 R₅ₑ;
R₅ₐ, R_{5c} and R_{5d} are each independently selected from hydrogen; —C₁₋₆alkyl; or —C₁₋₆alkyl substituted with 1, 2, 3, 4, 5 or 6 substituents selected from —F, —C₁₋₃alkyl, oxo, —OC₁₋₃alkyl, —NH₂, —NHC₁₋₃alkyl, —N(C₁₋₃alkyl)₂, —CN or 3-6 membered cycloalkyl;
R_{5b} and R₅ₑ are each independently selected from —F, —C₁₋₆alkyl, oxo, —OC₁₋₆alkyl, —NH₂, —NHC₁₋₆alkyl, —N(C₁₋₆alkyl)₂, —CN or 3-6 membered cycloalkyl, wherein, said —C₁₋₆alkyl is optionally substituted with 1, 2, 3 or 4 substituents selected from —F, —C₁₋₃alkyl, oxo, —OC₁₋₃alkyl, —NH₂, —NHC₁₋₃alkyl, —N(C₁₋₃alkyl)₂, —CN or 3-6 membered cycloalkyl;
R₁ is independently selected from —C₁₋₃alkyl; —C₁₋₃haloalkyl; 5 membered heteroaryl containing 1 or 2 heteroatoms selected from N, O or S; or —C₁₋₃alkyl substituted with 1, 2 or 3 substituents selected from —F, —OC₁₋₃alkyl, —NH₂, —NHC₁₋₃alkyl, —N(C₁₋₃alkyl)₂, —CN or 3-6 membered cycloalkyl; each of R₄ is independently selected from hydrogen, deuterium, —F, —Cl, —C₁₋₃alkyl, —OC₁₋₃alkyl, —NH₂, —NHC₁₋₃alkyl, —N(C₁₋₃alkyl)₂, —CN or 3-6 membered cycloalkyl;
R₁₁ and R₁₂ are independently selected from hydrogen or —C₁₋₃alkyl;
R₁₃ is selected from hydrogen or —C₁₋₃alkyl;
R₃ is independently selected from phenyl, said phenyl is independently optionally substituted with 1, 2, or 3 substituents selected from R_{3c};
R₃ₑ is selected from —F; —C₁₋₃alkyl; —OC₁₋₃alkyl; —NH₂; —NHC₁₋₃alkyl; —N(C₁₋₃alkyl)₂; —C(═O)NH₂, —C(═O)NH(C₁₋₃alkyl); —C(═O)N(C₁₋₃alkyl)₂; —CN; —S(═O)₂NH₂; —S(═O)₂NH(C₁₋₃alkyl); —S(═O)₂N(C₁₋₃alkyl)₂; —S(═O)₂C₁₋₃alkyl; —S(═O)(═NH)CH₃; or —S(═O)(═NCH₃)CH₃.

[34] The compound according to [33], wherein

R₅₁ is selected from

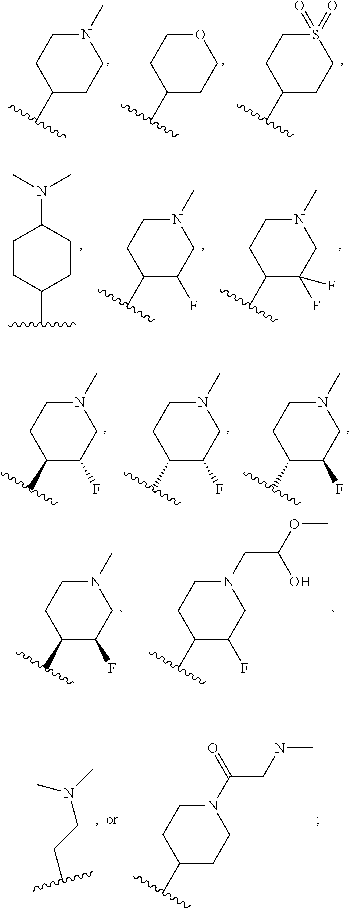

R₁ is independently selected from

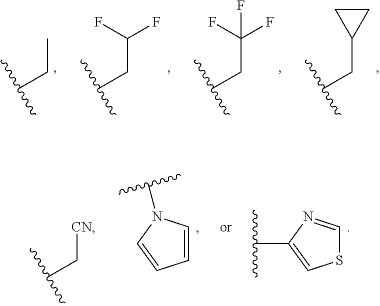

each of R₄ is hydrogen;
R₁₁ and R₁₂ are independently selected from hydrogen;
R₁₃ is selected from hydrogen;
R₃ is independently selected from

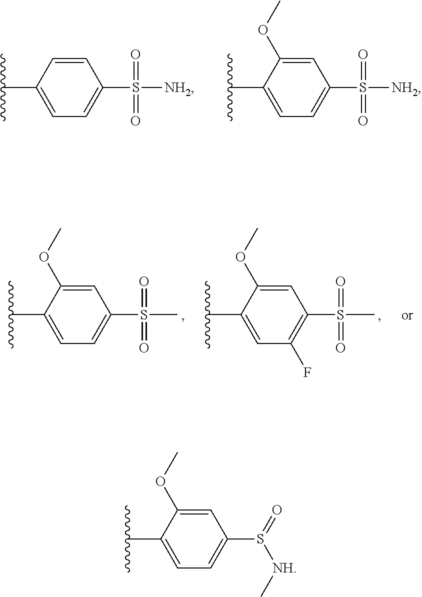

[35]. The compound according to any one of [1] to [34] wherein the formula (I) is:

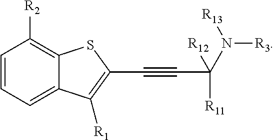

[36]. The compound according to [35], wherein R₁ is independently selected from —C₁₋₃haloalkyl; or —C₁₋₃alkyl optionally substituted with 1, 2 or 3 substituents selected from —F, —CN, —OC₁₋₃alkyl, —NH₂, —NHC₁₋₃alkyl, —N(C₁₋₃alkyl)₂ or 3-6 membered cycloalkyl such as cyclopropyl.

[37]. The compound according to [35] or [36], wherein R₁ is independently selected from

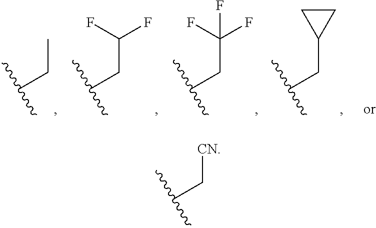

[38]. The compound according to any one of [35] to [37], wherein R₂ is —NHR₅₁.

[39]. The compound according to any one of [35] to [38], wherein R₅₁ is selected from —C₁₋₆alkyl, —C₁₋₆haloalkyl, 3-6 membered cycloalkyl, or 3-10 membered heterocycloalkyl; said —C₁₋₆alkyl, —C₁₋₆haloalkyl, 3-6 membered cycloalkyl, or 3-10 membered heterocycloalkyl are each independently optionally substituted with one or more (such as 1, 2, 3, 4, 5 or 6) substituents selected from halogen, —C₁₋₆alkyl (optionally substituted with one or more of halogen, —CN, oxo, ═NH, —OH, —OC₁₋₆alkyl, or 5-6 membered heterocycloalkyl optionally substituted with one or more of halogen, —CN, oxo, ═NH, —OH, or —OC₁₋₆alkyl), —C₂₋₆alkenyl, —C₂₋₆alkynyl, —C₁₋₆haloalkyl, —CN, —OH, —NH₂, oxo, ═NH, —OC₁₋₆alkyl, —SC₁₋₆alkyl, —C(O)C₁₋₆alkyl, —C(O)C₁₋₆alkyl-NHC₁₋₆alkyl, —C(O)C₁₋₆alkyl-N(C₁₋₆alkyl)₂, —C(O)NHC₁₋₆alkyl, —C(O)N(C₁₋₆alkyl)₂, —C(O)OC₁₋₆alkyl, —OC(O)C₁₋₆alkyl, —NHC₁₋₆alkyl, —N(C₁₋₆alkyl)₂, 3-6 membered cycloalkyl, or 3-12 membered heterocycloalkyl such as 5-12 membered spiro-heterocyclyl or bridged-heterocyclyl (optionally substituted with one or more of halogen, —CN, oxo, ═NH, —OH, or —OC₁₋₆alkyl), wherein said heterocycloalkyl independently contains one or more heteroatoms selected from N, O, or S.

[40]. The compound according to any one of [35] to [39], wherein R₅₁ is selected from —C₁₋₆alkyl, 3-6 membered cycloalkyl, or 5-10 membered heterocycloalkyl such as 5-10 membered spiro-heterocyclyl or bridged-heterocyclyl; said —C₁₋₆alkyl, 3-6 membered cycloalkyl, or 5-10 membered heterocycloalkyl are each independently optionally substituted with one or more (such as 1, 2, 3, 4, 5 or 6) substituents selected from halogen, —C₁₋₆alkyl (optionally substituted with one or more of halogen, —CN, oxo, ═NH, —OH, —OC₁₋₆alkyl, or

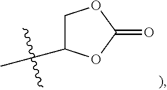 [SMILES: *C1COC(=O)O1]

—CN, —OH, NH₂, oxo, ═NH, —OC₁₋₆alkyl, —SC₁₋₆alkyl, —C(O)C₁₋₆alkyl-NHC₁₋₆alkyl, —C(O)C₁₋₆alkyl-N(C₁₋₆alkyl)₂, —NHC₁₋₆alkyl, —N(C₁₋₆alkyl)₂, 3-5 membered cycloalkyl, or 5-12 membered heterocycloalkyl such as 5-12 membered spiro-heterocyclyl or bridged-heterocyclyl (optionally substituted with one or more of halogen, —CN, oxo, ═NH, —OH, or —OC₁₋₆alkyl), wherein said heterocycloalkyl independently contains one or more heteroatoms selected from N, O, or S.

[41]. The compound according to any one of [35] to [40], wherein R₅₁ is selected from —C₁₋₃alkyl, cyclohexyl, or 6-8 membered heterocycloalkyl such as 6-8 membered spiro-heterocyclyl or bridged-heterocyclyl; said —C₁₋₃alkyl, cyclohexyl, or 6-8 membered heterocycloalkyl are each independently optionally substituted with one or more (such as 1, 2, 3, 4, 5 or 6) substituents selected from halogen, —C₁₋₆alkyl (optionally substituted with one or more of oxo, ═NH, —OH, —OC₁₋₆alkyl, or

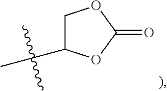 [SMILES: *C1COC(=O)O1]

—CN, —OH, NH₂, oxo, ═NH, —OC₁₋₆alkyl, —SC₁₋₆alkyl, —C(O)CH₂—NHC₁₋₆alkyl, —C(O)CH₂—N(C₁₋₆alkyl)₂, —NHC₁₋₆alkyl, —N(C₁₋₆alkyl)₂, cyclopropyl, or 5-12 membered heterocycloalkyl (such as

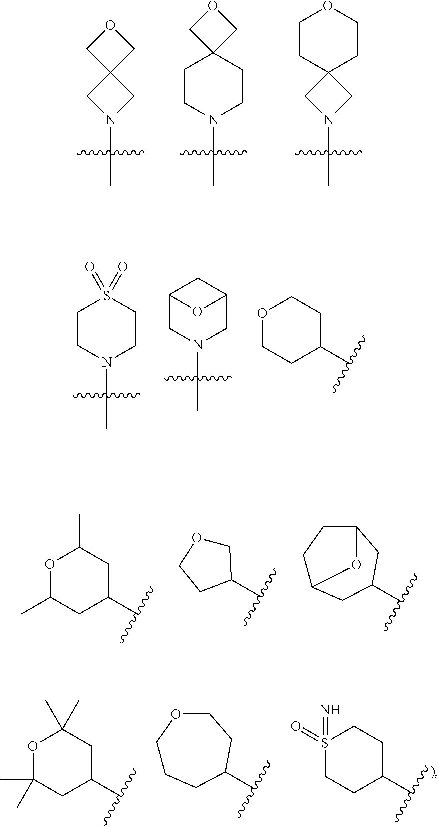

wherein said heterocycloalkyl independently contains one or more heteroatoms selected from N, O, or S, preferably R₅₁ is

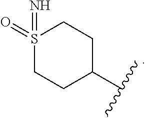 [SMILES: *C1CCS(=N)(=O)CC1]

[42]. The compound according to any one of [35] to [41], wherein R₂ is selected from:

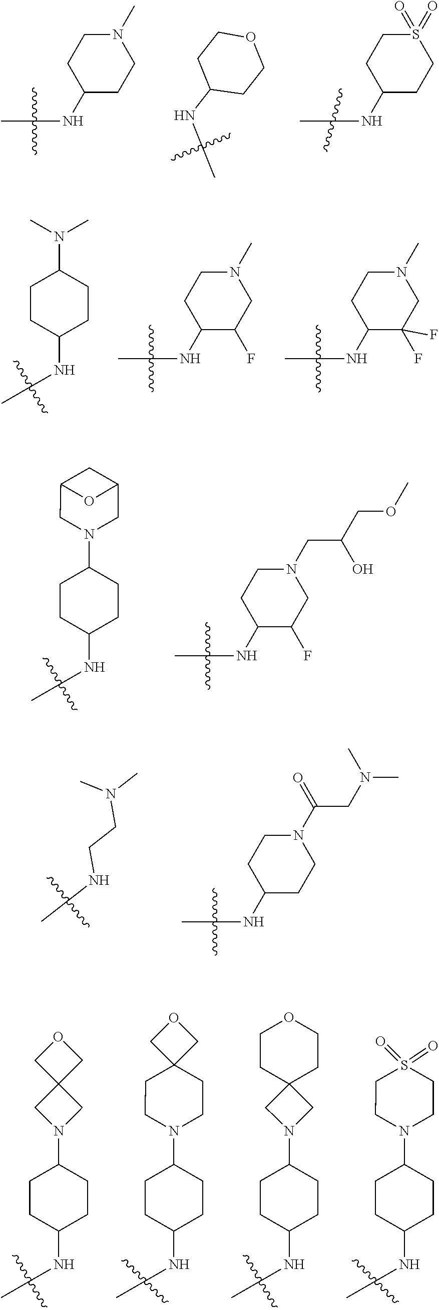

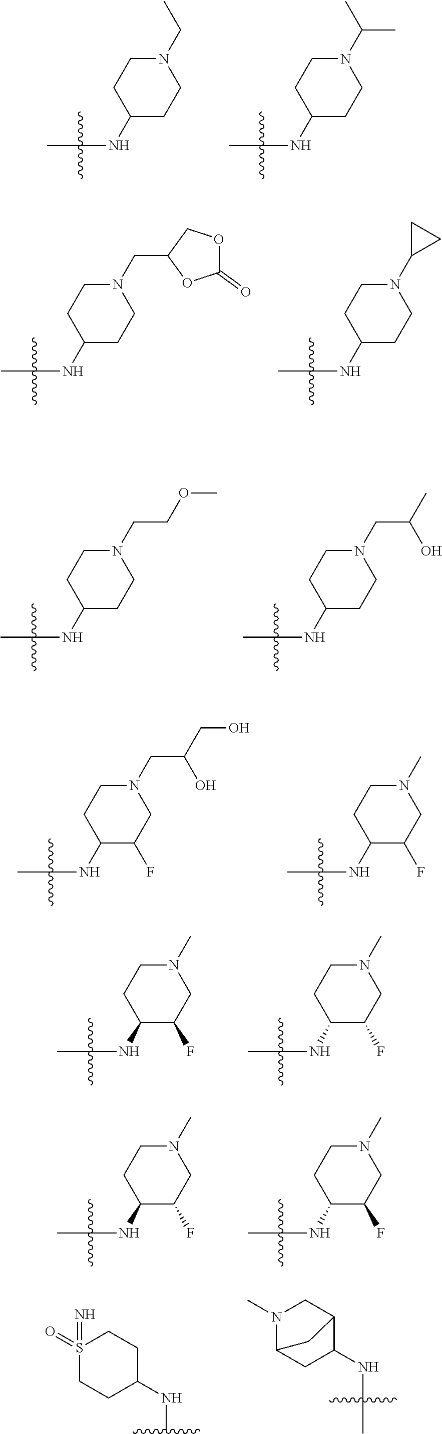

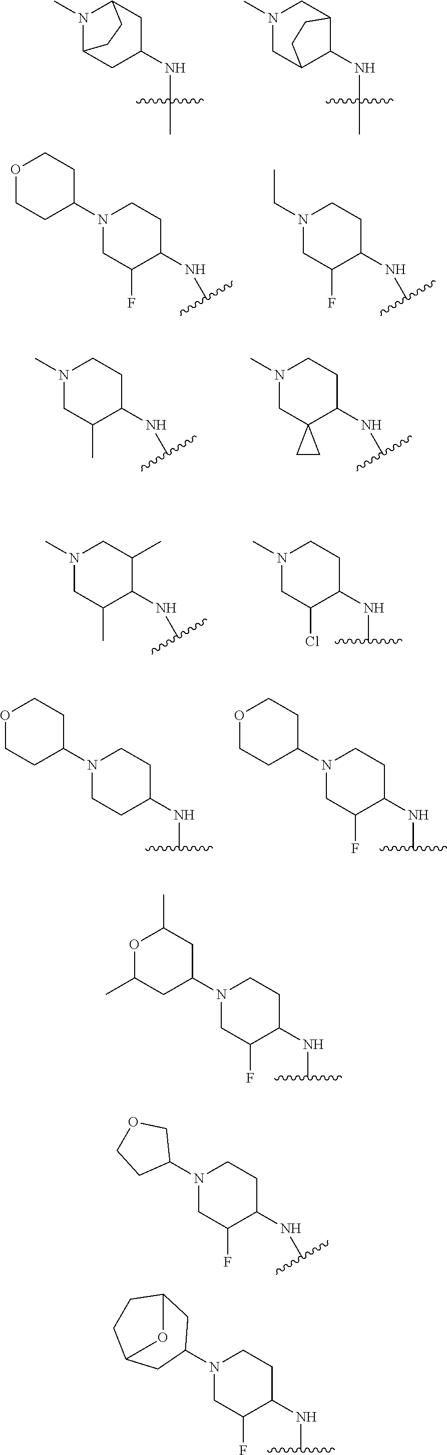

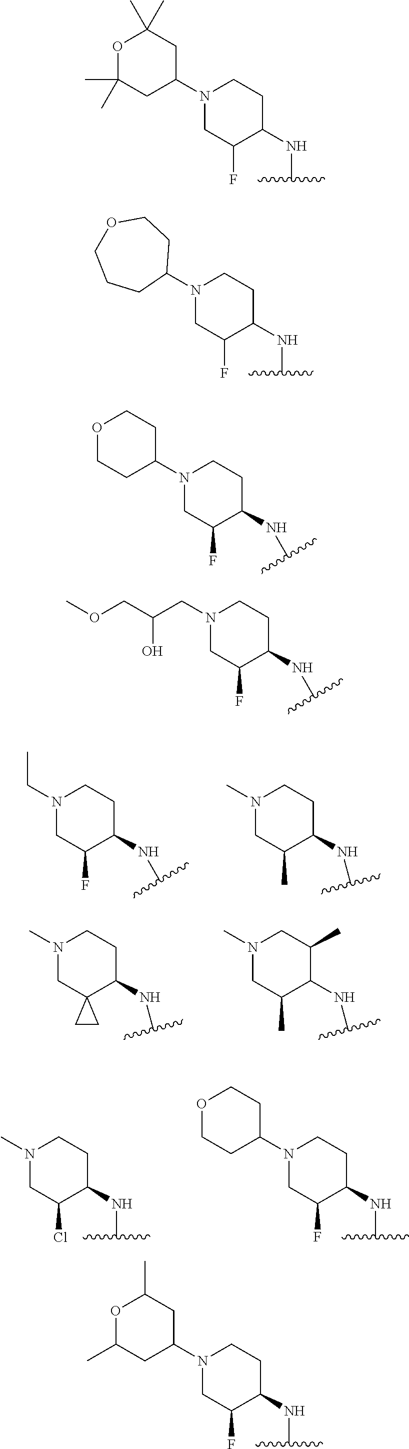

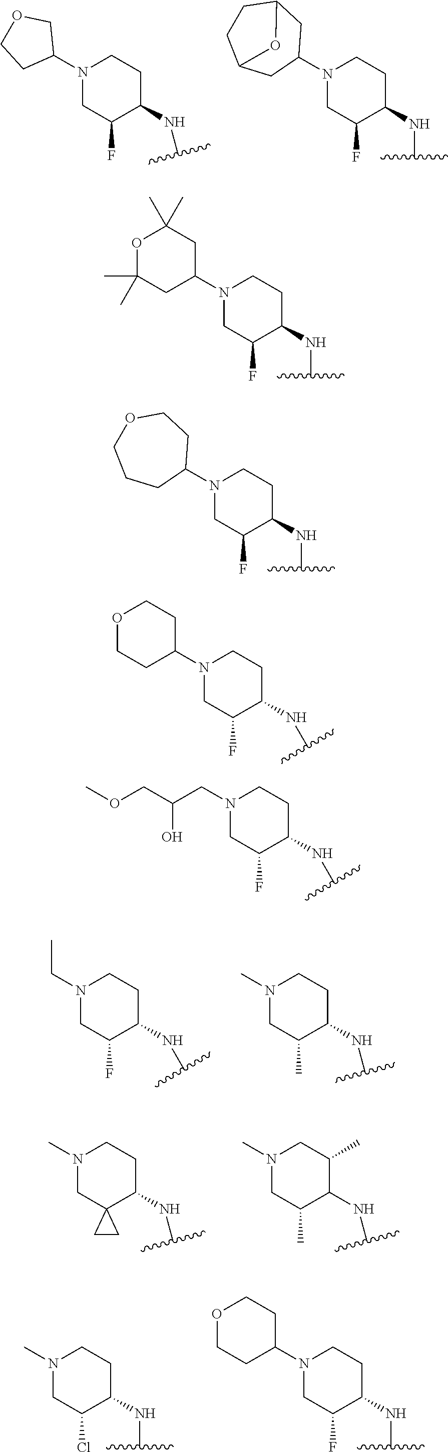

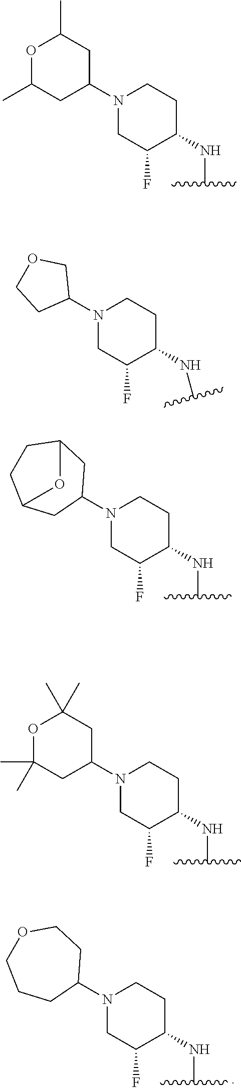 [SMILES: *N[C@H]1CCN(C2CC(C)OC(C)C2)C[C@H]1F ; *N[C@H]1CCN(C2CCOC2)C[C@H]1F ; *N[C@H]1CCN(C2CC3CCC(C2)O3)C[C@H]1F ; *N[C@H]1CCN(C2CC(C)(C)OC(C)(C)C2)C[C@H]1F ; *N[C@H]1CCN(C2CCCOCC2)C[C@H]1F]

[43]. The compound according to any one of [35] to [42], wherein R₃ is independently selected from phenyl, 5-10 membered heterocycloalkenyl containing 1 or 2 heteroatoms selected from N, O or S, or 5-10 membered, preferably 5-6 membered heteroaryl containing 1 or 2 heteroatoms selected from N, O or S; each of which is independently optionally substituted with 1, 2, or 3 substituents selected from R₃ₐ;
wherein R₃ₐ is selected from halogen; oxo; —C₁₋₃alkyl; —OC₁₋₃alkyl; —NH₂; —NHC₁₋₃alkyl; —N(C₁₋₃alkyl)₂; —C(═O)NH₂, —C(═O)NH(C₁₋₃alkyl); —C(═O)N(C₁₋₃alkyl)₂; —CN; —S(═O)₂NH₂; —S(═O)₂NH(C₁₋₃alkyl); —S(═O)₂NHC(═O)C₁₋₃alkyl; —S(═O)₂N(C₁₋₃alkyl)₂; —S(═O)₂C₁₋₃alkyl; —S(═O)(═NH)C₁₋₃alkyl; —S(═O)(═NH)C₃₋₆cycloalkyl; —S(═O)(═NR′)C₂₋₆heterocycloalkyl; —S(═O)(═NC₃₋₆cycloalkyl)C₁₋₃alkyl; —S(═O)(═NC₂₋₆heterocycloalkyl)C₁₋₃alkyl; —S(═O)(═NC₁₋₃alkyl)C₁₋₃alkyl; —S(═O)(═NCN)C₁₋₃alkyl; —N(C₁₋₃alkyl)S(═O)₂C₁₋₃alkyl; —PO(C₁₋₃alkyl)₂; 3-6 membered cycloalkyl; 3-6 membered heterocycloalkyl optionally containing 1, 2 or 3 heteroatoms selected from N, O, P or S; wherein said C₁₋₃alkyl, OC₁₋₃alkyl, 3-6 membered cycloalkyl, C₃₋₆cycloalkyl, C₂₋₆heterocycloalkyl or 3-6 membered heterocycloalkyl at each occurrence is independently optionally substituted with 1, 2, 3, 4, 5 or 6 substituents selected from deuterium, halogen, —CN, —C₁₋₃alkyl, oxo, —OH, —OC₁₋₃alkyl, —NH₂, —NHC₁₋₃alkyl, —N(C₁₋₃alkyl)₂, —COOH, —C(O)OC₁₋₃alkyl, —C(═O)NH₂, —C(═O)NH(C₁₋₃alkyl), —C(═O)N(C₁₋₃alkyl)₂, —S(═O)₂NH₂, —S(═O)₂NH(C₁₋₃alkyl), —S(═O)₂C₁₋₃alkyl, —S(═O)₂N(C₁₋₃alkyl)₂, —S(═O)(═NH)C₁₋₃alkyl, —S(═O)(═NC₁₋₃alkyl)C₁₋₃alkyl, or 3-6 membered cycloalkyl.

[44]. The compound according to [43], wherein R₃ is independently selected from phenyl, pyridinyl, and 5-10 membered benzoheterocycloalkyl containing 1 or 2 heteroatoms selected from N, O or S, each of which is optionally substituted with 1, 2, or 3 substituents selected from R₃ₐ;
wherein R₃ₐ is selected from deuterium, —F; oxo; —OC₁₋₃alkyl; —C(═O)NH₂, —C(═O)NH(C₁₋₃alkyl); —CN; —S(═O)₂NH₂; —S(═O)₂NH(C₁₋₃alkyl); —S(═O)₂NHC(═O)C₁₋₃alkyl; —S(═O)₂N(C₁₋₃alkyl)₂; —S(═O)₂C₁₋₃alkyl; —S(═O)(═NH)C₁₋₃alkyl; —S(═O)(═NC₁₋₃alkyl)C₁₋₃alkyl; —N(C₁₋₃alkyl)S(═O)₂C₁₋₃alkyl; —PO(C₁₋₃alkyl)₂; or 3-6 membered heterocycloalkyl optionally containing 1, 2 or 3 heteroatoms selected from N, O, P or S; wherein said C₁₋₃alkyl, OC₁₋₃alkyl or 3-6 membered heterocycloalkyl at each occurrence is independently optionally substituted with 1, 2, 3, 4, 5 or 6 substituents selected from deuterium, —F, —CN, oxo, —C₁₋₃alkyl, —OH, —OC₁₋₃alkyl, —N(C₁₋₃alkyl)₂, —C(O)OC₁₋₃alkyl, —C(═O)NH₂, —C(═O)NH(C₁₋₃alkyl), or —C(═O)N(C₁₋₃alkyl)₂.

[45]. The compound according to [44], wherein R₃ is independently selected from phenyl, pyridinyl,

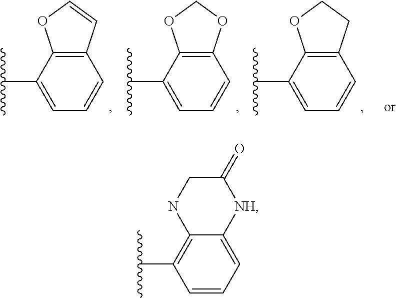

each of which is optionally substituted with 1, 2, 3 substituents selected from R₃ₐ;
wherein R₃ₐ is selected from —F; oxo; —OC₁₋₃alkyl; —C(═O)NHC₁₋₃alkyl; —S(═O)₂NH₂; —S(═O)₂NHC(═O)CH₃; —S(═O)₂CH₃; —S(═O)(═NH)C₁₋₃alkyl; —S(═O)(═NC₁₋₃alkyl)C₁₋₃alkyl; —N(CH₃)S(═O)₂CH₃; —PO(C₁₋₃alkyl)₂; morpholinyl or 5-6 membered heterocycloalkyl optionally containing 1, 2 or 3 heteroatoms selected from N, O, or P; wherein said C₁₋₃alkyl, OC₁₋₃alkyl or 5-6 membered heterocycloalkyl at each occurrence is independently optionally substituted with 1, 2, 3, 4, 5 or 6 substituents selected from deuterium, —F, —CN, oxo, —OH, —OCH₃, —N(C₁₋₃alkyl)₂, —C(O)OCH₃, or —C(═O)NH₂.

[46]. The compound according to [43], wherein R₃ is independently selected from phenyl optionally substituted with 1, 2, or 3 substituents selected from R₃ₐ;
wherein R₃ₐ is selected from: —F, —OCH₃, —OCD₃, —OCH₂CN, —OCH₂CF₃, —CH₂F, —CHF₂, —OCH₂CH₂OH, —OCH₂CH₂OCH₃, —S(O)₂NH₂, —S(O)₂NHCOCH₃, —S(O)₂CH₃, —S(═O)(═NH)CH₃, —S(═O)(═NCH₃)CH₃, —N(CH₃)S(═O)₂CH₃, —C(O)NHCH₃, —C(O)NHCH(COOCH₃)CH₂CH₂CONH₂, —PO(CH₃)₂, morpholinyl, or

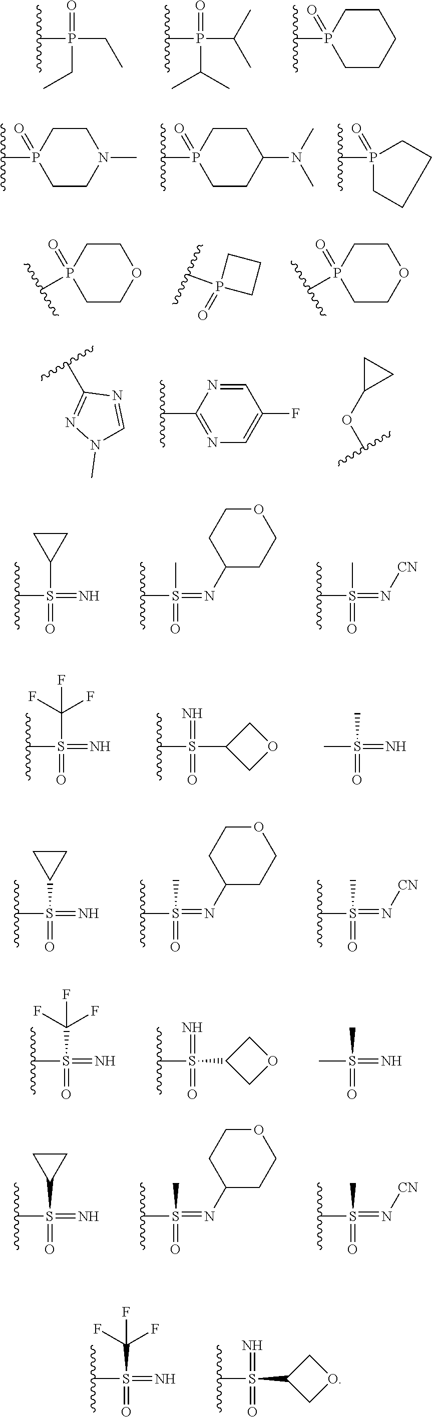

[47]. The compound according to any one of [1] to [46], wherein both of R₁₁ and R₁₂ are hydrogen.

[48]. The compound according to any one of [1] to [47], wherein R₁₃ is H.

[49]. The compound according to any one of [1] to [48], wherein the chemical moiety

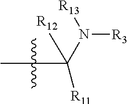

is selected from:

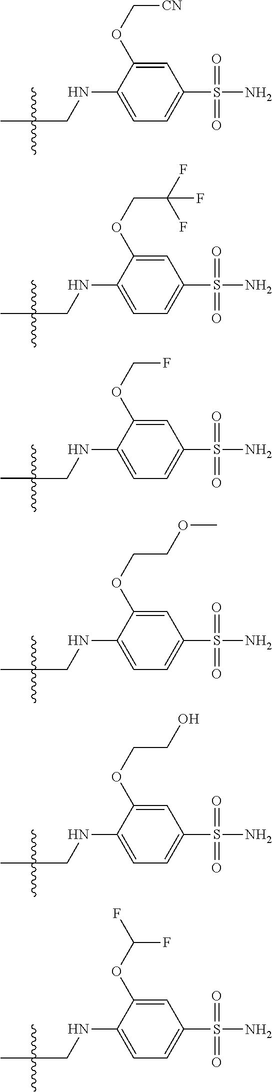

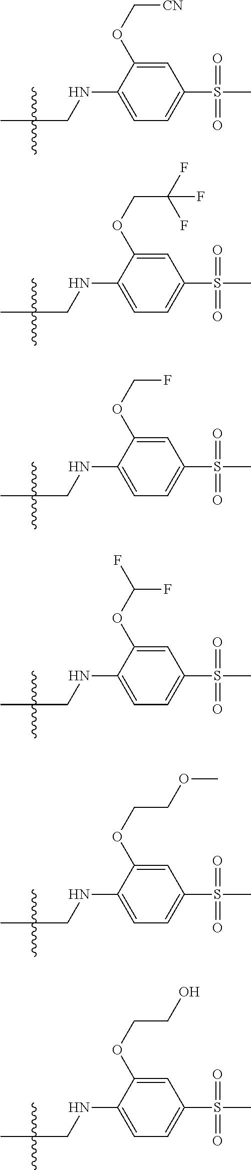 [SMILES: *CNc1ccc(S(C)(=O)=O)cc1OCC#N ; *CNc1ccc(S(C)(=O)=O)cc1OCC(F)(F)F ; *CNc1ccc(S(C)(=O)=O)cc1OCF ; *CNc1ccc(S(C)(=O)=O)cc1OC(F)F ; *CNc1ccc(S(C)(=O)=O)cc1OCCOC ; *CNc1ccc(S(C)(=O)=O)cc1OCCO]

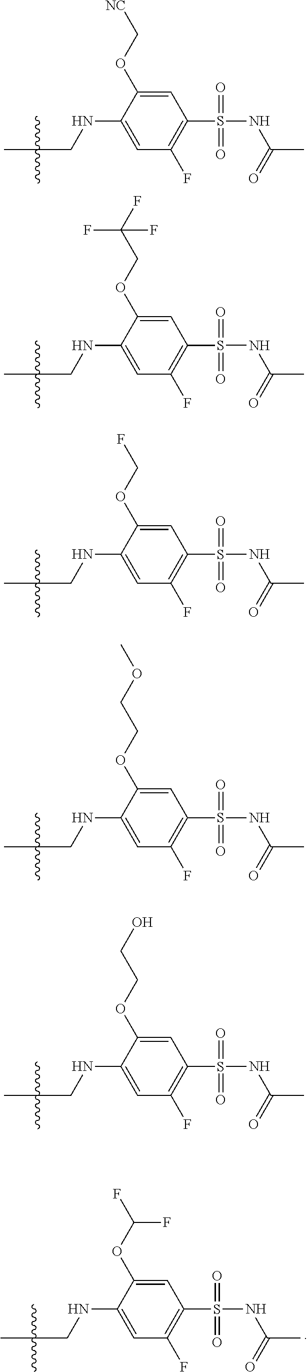 [SMILES: *CNc1cc(F)c(S(=O)(=O)NC(C)=O)cc1OCC#N ; *CNc1cc(F)c(S(=O)(=O)NC(C)=O)cc1OCC(F)(F)F ; *CNc1cc(F)c(S(=O)(=O)NC(C)=O)cc1OCF ; *CNc1cc(F)c(S(=O)(=O)NC(C)=O)cc1OCCOC ; *CNc1cc(F)c(S(=O)(=O)NC(C)=O)cc1OCCO ; *CNc1cc(F)c(S(=O)(=O)NC(C)=O)cc1OC(F)F]

[50]. The compound according to any one of [1] to [48], wherein the chemical moiety

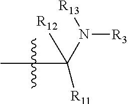

is selected from:

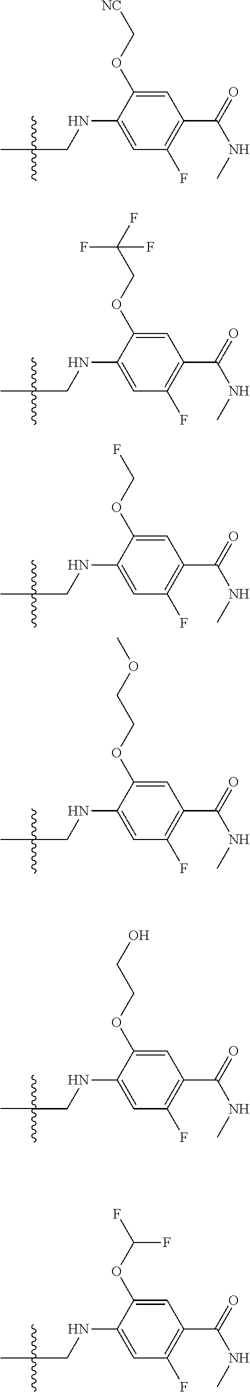 [SMILES: *CNc1cc(F)c(C(=O)NC)cc1OCC#N ; *CNc1cc(F)c(C(=O)NC)cc1OCC(F)(F)F ; *CNc1cc(F)c(C(=O)NC)cc1OCF ; *CNc1cc(F)c(C(=O)NC)cc1OCCOC ; *CNc1cc(F)c(C(=O)NC)cc1OCCO ; *CNc1cc(F)c(C(=O)NC)cc1OC(F)F]

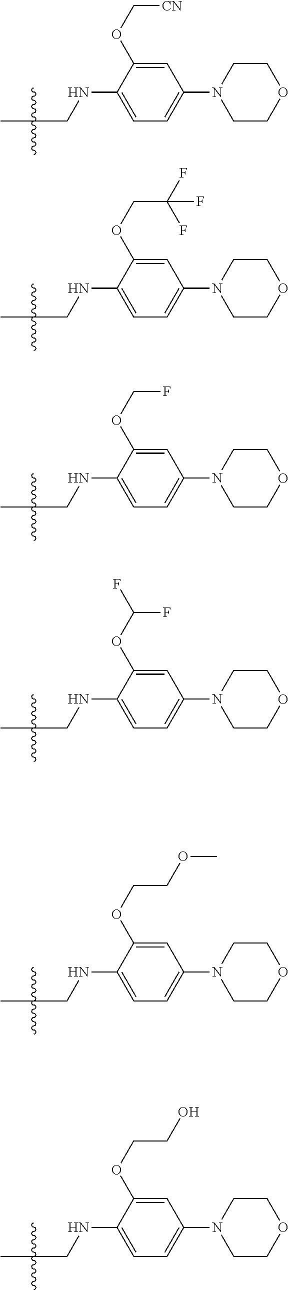 [SMILES: *CNc1ccc(N2CCOCC2)cc1OCC#N ; *CNc1ccc(N2CCOCC2)cc1OCC(F)(F)F ; *CNc1ccc(N2CCOCC2)cc1OCF ; *CNc1ccc(N2CCOCC2)cc1OC(F)F ; *CNc1ccc(N2CCOCC2)cc1OCCOC ; *CNc1ccc(N2CCOCC2)cc1OCCO]

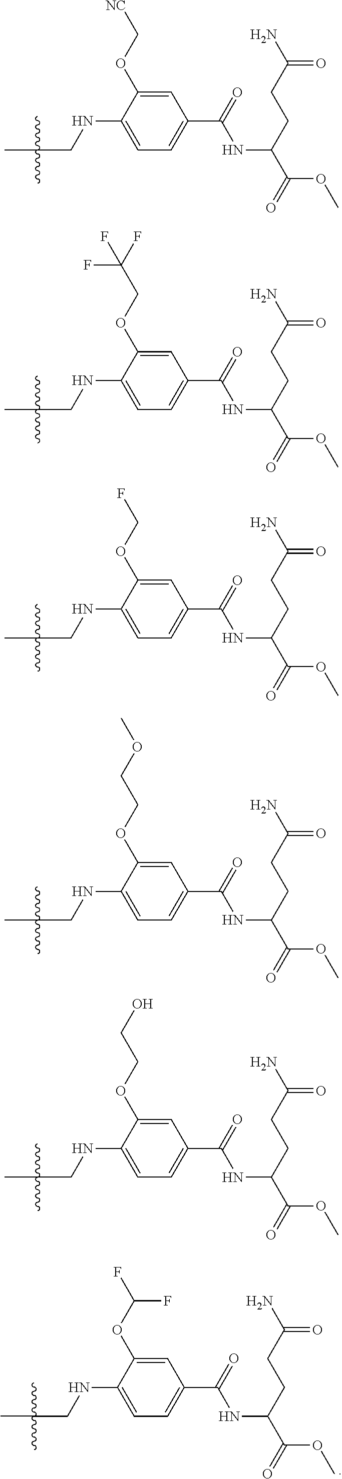 [SMILES: *CNc1ccc(C(=O)NC(CCC(N)=O)C(=O)OC)cc1OCC#N ; *CNc1ccc(C(=O)NC(CCC(N)=O)C(=O)OC)cc1OCC(F)(F)F ; *CNc1ccc(C(=O)NC(CCC(N)=O)C(=O)OC)cc1OCF ; *CNc1ccc(C(=O)NC(CCC(N)=O)C(=O)OC)cc1OCCOC ; *CNc1ccc(C(=O)NC(CCC(N)=O)C(=O)OC)cc1OCCO ; *CNc1ccc(C(=O)NC(CCC(N)=O)C(=O)OC)cc1OC(F)F]

[51]. The compound according to any one of [1] to [48], wherein the chemical moiety

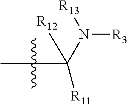

is selected from:

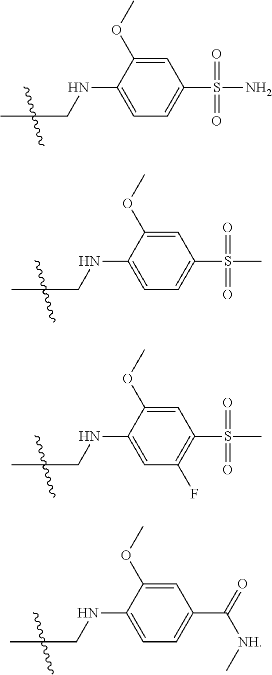 [SMILES: *CNc1ccc(S(N)(=O)=O)cc1OC ; *CNc1ccc(S(C)(=O)=O)cc1OC ; *CNc1cc(F)c(S(C)(=O)=O)cc1OC ; *CNc1ccc(C(=O)NC)cc1OC]

[52]. The compound according to any one of [1] to [48], wherein the chemical moiety

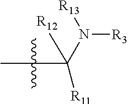

is selected from:

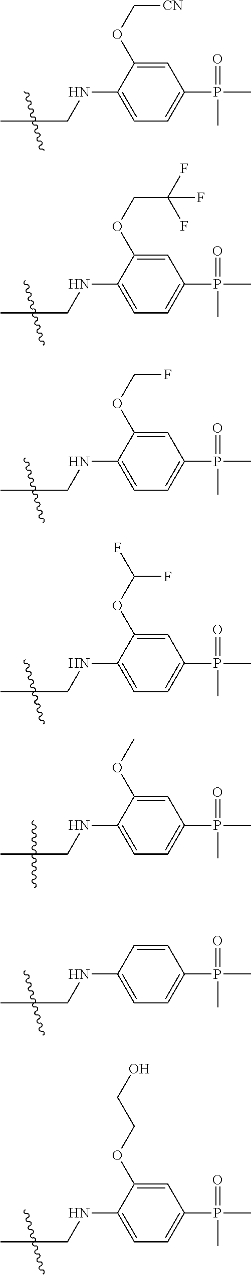

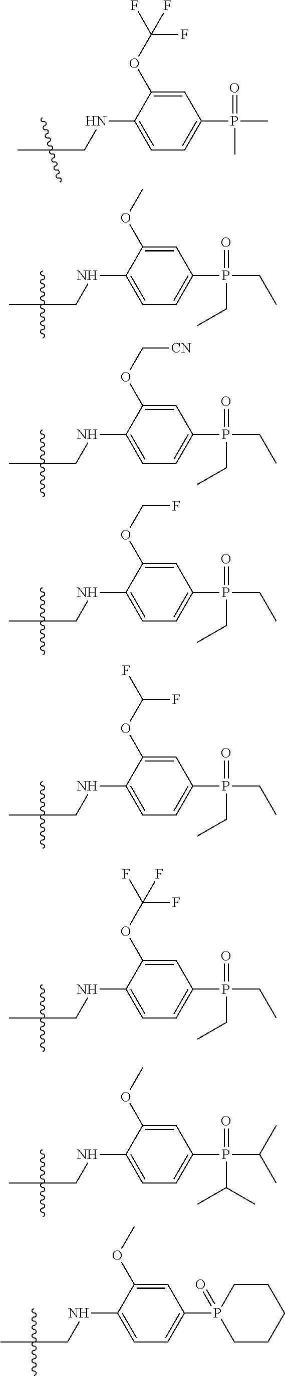

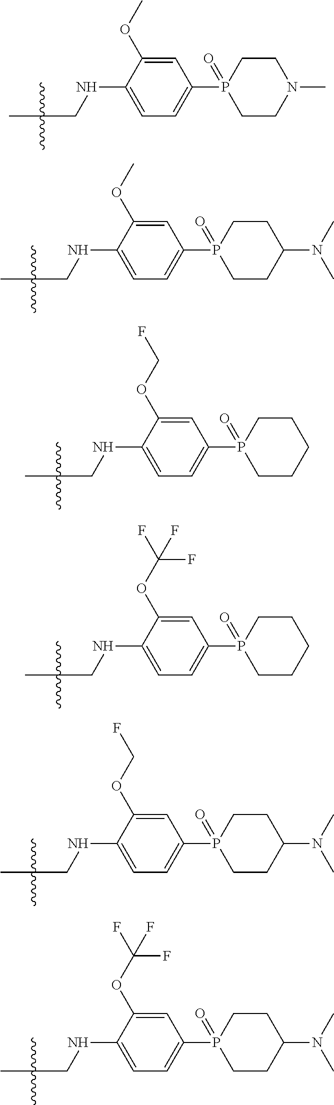 [SMILES: *CNc1ccc(P2(=O)CCN(C)CC2)cc1OC ; *CNc1ccc(P2(=O)CCC(N(C)C)CC2)cc1OC ; *CNc1ccc(P2(=O)CCCCC2)cc1OCF ; *CNc1ccc(P2(=O)CCCCC2)cc1OC(F)(F)F ; *CNc1ccc(P2(=O)CCC(N(C)C)CC2)cc1OCF ; *CNc1ccc(P2(=O)CCC(N(C)C)CC2)cc1OC(F)(F)F]

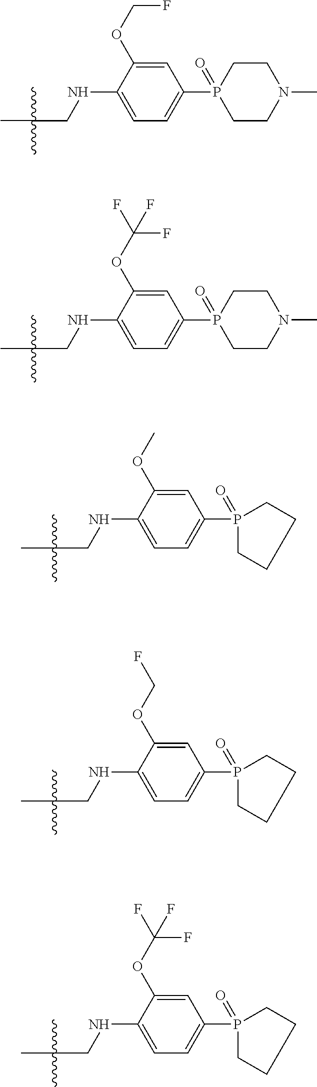 [SMILES: *CNc1ccc(P2(=O)CCN(C)CC2)cc1OCF ; *CNc1ccc(P2(=O)CCN(C)CC2)cc1OC(F)(F)F ; *CNc1ccc(P2(=O)CCCC2)cc1OC ; *CNc1ccc(P2(=O)CCCC2)cc1OCF ; *CNc1ccc(P2(=O)CCCC2)cc1OC(F)(F)F]

[53]. The compound according to any one of [1] to [48], wherein the chemical moiety

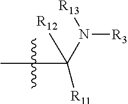

selected from:

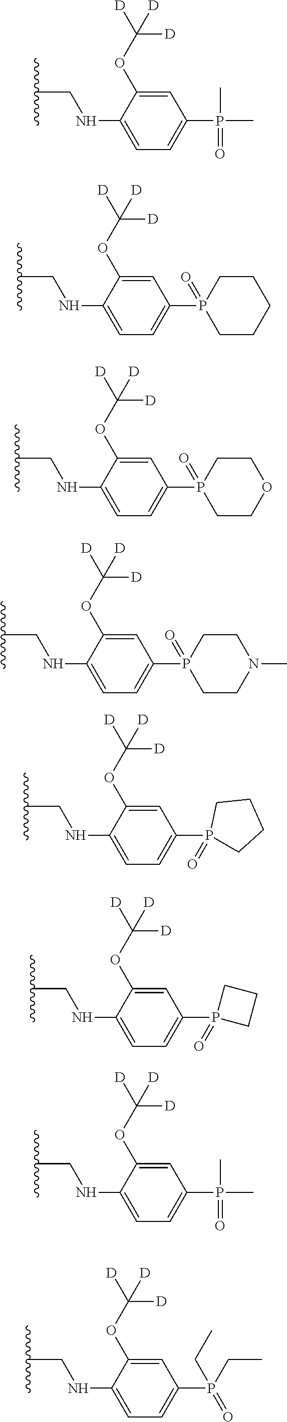

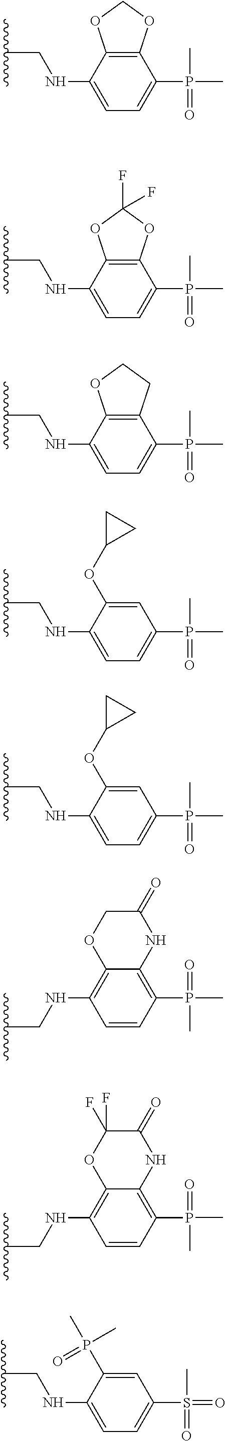

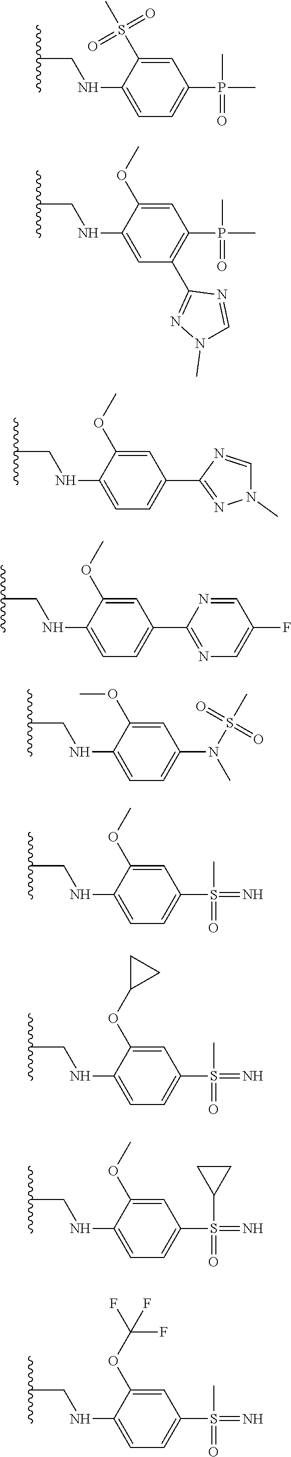

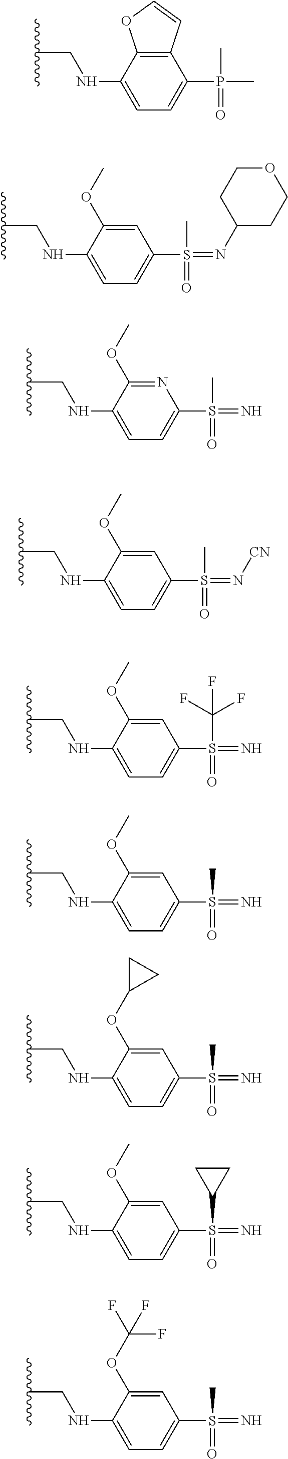

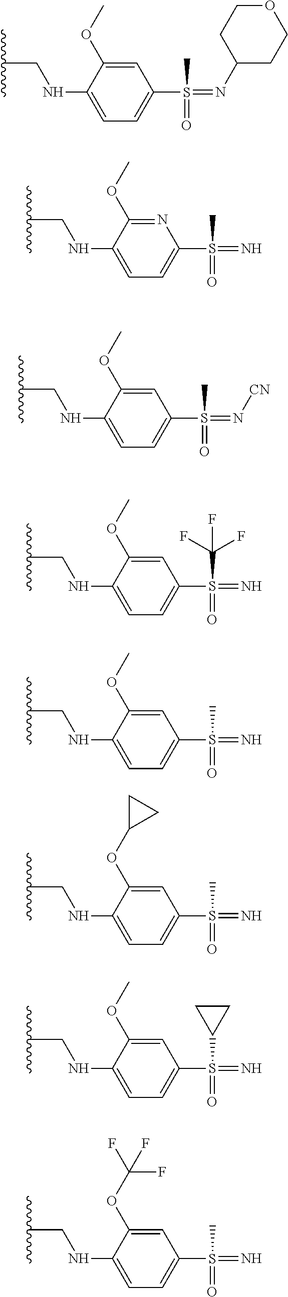

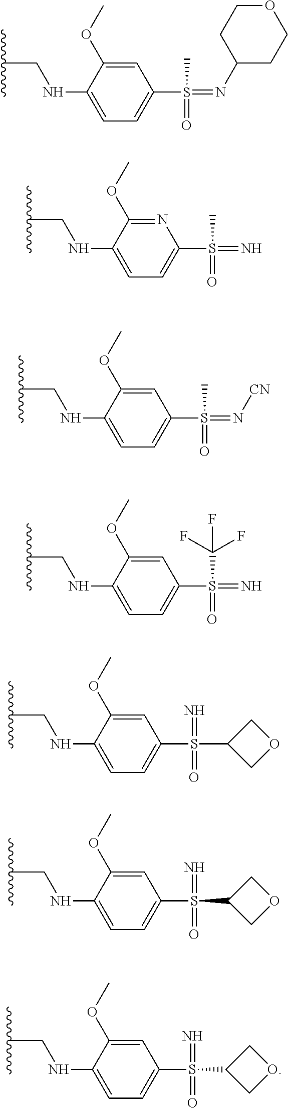

[54] The compound according to any one of [1] to [53], wherein the compound of formula (I) is selected from:

| No. | IUPAC Name |
|---|---|
| 1 | 4-((3-(3-(cyanomethyl)-7-((3,3-difluoro-1-methylpiperidin-4-yl)amino)benzo[b]thiophen-2-yl)prop-2-yn-1- |
| | yl)amino)-3-methoxybenzenesulfonamide; |
| 2 | 4-((3-(7-((3,3-difluoro-1-methylpiperidin-4-yl)amino)-3-(thiazol-4-yl)benzo[b]thiophen-2-yl)prop-2-yn-1- |
| | yl)amino)-3-methoxybenzenesulfonamide; |
| 3 | 4-((3-(7-((3,3-difluoro-1-methylpiperidin-4-yl)amino)-3-(2,2,2-trifluoroethyl)benzo[b]thiophen-2-yl)prop-2- |
| | yn-1-yl)amino)-3-methoxybenzenesulfonamide; |
| 4 | 4-((3-(3-(cyclopropylmethyl)-7-((3,3-difluoro-1-methylpiperidin-4-yl)amino)benzo[b]thiophen-2-yl)prop-2- |
| | yn-1-yl)amino)-3-methoxybenzenesulfonamide; |
| 5 | 4-((3-(7-((3,3-difluoro-1-methylpiperidin-4-yl)amino)-3-(2,2-difluoroethyl)benzo[b]thiophen-2-yl)prop-2- |
| | yn-1-yl)amino)-3-methoxybenzenesulfonamide; |
| 6 | 4-((3-(7-((3,3-difluoro-1-methylpiperidin-4-yl)amino)-3-(1H-pyrrol-1-yl)benzo[b]thiophen-2-yl)prop-2-yn- |
| | 1-yl)amino)-3-methoxybenzenesulfonamide; |
| 7 | 4-((3-(7-((3,3-difluoro-1-methylpiperidin-4-yl)amino)-3-ethylbenzo[b]thiophen-2-yl)prop-2-yn-1-yl)amino)- |
| | 3-methoxybenzenesulfonamide; |
| 8 | 4-((3-(3-(cyanomethyl)-7-((1,1-dioxidotetrahydro-2H-thiopyran-4-yl)amino)benzo[b]thiophen-2-yl)prop-2- |
| | yn-1-yl)amino)-3-methoxybenzenesulfonamide; |
| 9 | 4-((3-(7-((1,1-dioxidotetrahydro-2H-thiopyran-4-yl)amino)-3-(thiazol-4-yl)benzo[b]thiophen-2-yl)prop-2-yn- |
| | 1-yl)amino)-3-methoxybenzenesulfonamide; |
| 10 | 4-((3-(7-((1,1-dioxidotetrahydro-2H-thiopyran-4-yl)amino)-3-(2,2,2-trifluoroethyl)benzo[b]thiophen-2-yl)prop- |
| | 2-yn-1-yl)amino)-3-methoxybenzenesulfonamide; |
| 11 | 4-((3-(3-(cyclopropylmethyl)-7-((1,1-dioxidotetrahydro-2H-thiopyran-4-yl)amino)benzo[b]thiophen-2-yl)prop- |
| | 2-yn-1-yl)amino)-3-methoxybenzenesulfonamide; |
| 12 | 4-((3-(3-(2,2-difluoroethyl)-7-((1,1-dioxidotetrahydro-2H-thiopyran-4-yl)amino)benzo[b]thiophen-2-yl)prop- |
| | 2-yn-1-yl)amino)-3-methoxybenzenesulfonamide; |
| 13 | 4-((3-(7-((1,1-dioxidotetrahydro-2H-thiopyran-4-yl)amino)-3-(1H-pyrrol-1-yl)benzo[b]thiophen-2-yl)prop- |
| | 2-yn-1-yl)amino)-3-methoxybenzenesulfonamide; |
| 14 | 4-((3-(7-((1,1-dioxidotetrahydro-2H-thiopyran-4-yl)amino)-3-ethylbenzo[b]thiophen-2-yl)prop-2-yn-1-yl)amino)- |
| | 3-methoxybenzenesulfonamide; |
| 15 | 4-((3-(3-(cyanomethyl)-7-(((3R,4S)-3-fluoro-1-methylpiperidin-4-yl)amino)benzo[b]thiophen-2-yl)prop-2- |
| | yn-1-yl)amino)-3-methoxybenzenesulfonamide; |
| 16 | 4-((3-(7-(((3R,4S)-3-fluoro-1-methylpiperidin-4-yl)amino)-3-(thiazol-4-yl)benzo[b]thiophen-2-yl)prop-2- |
| | yn-1-yl)amino)-3-methoxybenzenesulfonamide; |
| 17 | 4-((3-(7-(((3R,4S)-3-fluoro-1-methylpiperidin-4-yl)amino)-3-(2,2,2-trifluoroethyl)benzo[b]thiophen-2-yl)prop- |
| | 2-yn-1-yl)amino)-3-methoxybenzenesulfonamide; |
| 18 | 4-((3-(3-(cyclopropylmethyl)-7-(((3R,4S)-3-fluoro-1-methylpiperidin-4-yl)amino)benzo[b]thiophen-2-yl)prop- |
| | 2-yn-1-yl)amino)-3-methoxybenzenesulfonamide; |
| 19 | 4-((3-(3-(2,2-difluoroethyl)-7-(((3R,4S)-3-fluoro-1-methylpiperidin-4-yl)amino)benzo[b]thiophen-2-yl)prop- |
| | 2-yn-1-yl)amino)-3-methoxybenzenesulfonamide; |
| 20 | 4-((3-(7-(((3R,4S)-3-fluoro-1-methylpiperidin-4-yl)amino)-3-(1H-pyrrol-1-yl)benzo[b]thiophen-2-yl)prop-2- |
| | yn-1-yl)amino)-3-methoxybenzenesulfonamide; |
| 21 | 4-((3-(3-ethyl-7-(((3R,4S)-3-fluoro-1-methylpiperidin-4-yl)amino)benzo[b]thiophen-2-yl)prop-2-yn-1-yl)amino)- |
| | 3-methoxybenzenesulfonamide; |
| 22 | 4-((3-(3-(cyanomethyl)-7-((3-fluoro-1-methylpiperidin-4-yl)amino)benzo[b]thiophen-2-yl)prop-2-yn-1-yl)amino)- |
| | 3-methoxybenzenesulfonamide; |
| 23 | 4-((3-(7-((3-fluoro-1-methylpiperidin-4-yl)amino)-3-(thiazol-4-yl)benzo[b]thiophen-2-yl)prop-2-yn-1-yl)amino)- |
| | 3-methoxybenzenesulfonamide; |
| 24 | 4-((3-(7-((3-fluoro-1-methylpiperidin-4-yl)amino)-3-(2,2,2-trifluoroethyl)benzo[b]thiophen-2-yl)prop-2-yn- |
| | 1-yl)amino)-3-methoxybenzenesulfonamide; |
| 25 | 4-((3-(3-(cyclopropylmethyl)-7-((3-fluoro-1-methylpiperidin-4-yl)amino)benzo[b]thiophen-2-yl)prop-2-yn- |
| | 1-yl)amino)-3-methoxybenzenesulfonamide; |
| 26 | 4-((3-(3-(2,2-difluoroethyl)-7-((3-fluoro-1-methylpiperidin-4-yl)amino)benzo[b]thiophen-2-yl)prop-2-yn-1- |
| | yl)amino)-3-methoxybenzenesulfonamide; |
| 27 | 4-((3-(7-((3-fluoro-1-methylpiperidin-4-yl)amino)-3-(1H-pyrrol-1-yl)benzo[b]thiophen-2-yl)prop-2-yn-1- |
| | yl)amino)-3-methoxybenzenesulfonamide; |
| 28 | 4-((3-(3-ethyl-7-((3-fluoro-1-methylpiperidin-4-yl)amino)benzo[b]thiophen-2-yl)prop-2-yn-1-yl)amino)-3- |
| | methoxybenzenesulfonamide; |
| 29 | 4-((3-(3-(cyanomethyl)-7-((1-(dimethylglycyl)piperidin-4-yl)amino)benzo[b]thiophen-2-yl)prop-2-yn-1-yl) |
| | amino)-3-methoxybenzenesulfonamide; |
| 30 | 4-((3-(7-((1-(dimethylglycyl)piperidin-4-yl)amino)-3-(thiazol-4-yl)benzo[b]thiophen-2-yl)prop-2-yn-1-yl)amino)- |
| | 3-methoxybenzenesulfonamide; |
| 31 | 4-((3-(7-((1-(dimethylglycyl)piperidin-4-yl)amino)-3-(2,2,2-trifluoroethyl)benzo[b]thiophen-2-yl)prop-2-yn- |
| | 1-yl)amino)-3-methoxybenzenesulfonamide; |
| 32 | 4-((3-(3-(cyclopropylmethyl)-7-((1-(dimethylglycyl)piperidin-4-yl)amino)benzo[b]thiophen-2-yl)prop-2-yn- |
| | 1-yl)amino)-3-methoxybenzenesulfonamide; |
| 33 | 4-((3-(3-(2,2-difluoroethyl)-7-((1-(dimethylglycyl)piperidin-4-yl)amino)benzo[b]thiophen-2-yl)prop-2-yn-1- |
| | yl)amino)-3-methoxybenzenesulfonamide; |
| 34 | 4-((3-(7-((1-(dimethylglycyl)piperidin-4-yl)amino)-3-(1H-pyrrol-1-yl)benzo[b]thiophen-2-yl)prop-2-yn-1- |
| | yl)amino)-3-methoxybenzenesulfonamide; |
| 35 | 4-((3-(7-((1-(dimethylglycyl)piperidin-4-yl)amino)-3-ethylbenzo[b]thiophen-2-yl)prop-2-yn-1-yl)amino)-3- |
| | methoxybenzenesulfonamide; |
| 36 | 4-((3-(3-(cyanomethyl)-7-((3-fluoro-1-(2-hydroxy-3-methoxypropyl)piperidin-4-yl)amino)benzo[b]thiophen- |
| | 2-yl)prop-2-yn-1-yl)amino)-3-methoxybenzenesulfonamide; |
| 37 | 4-((3-(7-((3-fluoro-1-(2-hydroxy-3-methoxypropyl)piperidin-4-yl)amino)-3-(thiazol-4-yl)benzo[b]thiophen- |
| | 2-yl)prop-2-yn-1-yl)amino)-3-methoxybenzenesulfonamide; |
| 38 | 4-((3-(7-((3-fluoro-1-(2-hydroxy-3-methoxypropyl)piperidin-4-yl)amino)-3-(2,2,2-trifluoroethyl)benzo[b]thiophen- |
| | 2-yl)prop-2-yn-1-yl)amino)-3-methoxybenzenesulfonamide; |
| 39 | 4-((3-(3-(cyclopropylmethyl)-7-((3-fluoro-1-(2-hydroxy-3-methoxypropyl)piperidin-4-yl)amino)benzo[b]thiophen- |
| | 2-yl)prop-2-yn-1-yl)amino)-3-methoxybenzenesulfonamide; |
| 40 | 4-((3-(3-(2,2-difluoroethyl)-7-((3-fluoro-1-(2-hydroxy-3-methoxypropyl)piperidin-4-yl)amino)benzo[b]thiophen- |
| | 2-yl)prop-2-yn-1-yl)amino)-3-methoxybenzenesulfonamide; |
| 41 | 4-((3-(7-((3-fluoro-1-(2-hydroxy-3-methoxypropyl)piperidin-4-yl)amino)-3-(1H-pyrrol-1-yl)benzo[b]thiophen- |
| | 2-yl)prop-2-yn-1-yl)amino)-3-methoxybenzenesulfonamide; |
| 42 | 4-((3-(3-ethyl-7-((3-fluoro-1-(2-hydroxy-3-methoxypropyl)piperidin-4-yl)amino)benzo[b]thiophen-2-yl)prop- |
| | 2-yn-1-yl)amino)-3-methoxybenzenesulfonamide; |
| 43 | 4-((3-(3-(cyanomethyl)-7-(((3R,4R)-3-fluoro-1-methylpiperidin-4-yl)amino)benzo[b]thiophen-2-yl)prop-2-yn- |
| | 1-yl)amino)-3-methoxybenzenesulfonamide; |
| 44 | 4-((3-(7-(((3R,4R)-3-fluoro-1-methylpiperidin-4-yl)amino)-3-(thiazol-4-yl)benzo[b]thiophen-2-yl)prop-2-yn- |
| | 1-yl)amino)-3-methoxybenzenesulfonamide; |
| 45 | 4-((3-(7-(((3R,4R)-3-fluoro-1-methylpiperidin-4-yl)amino)-3-(2,2,2-trifluoroethyl)benzo[b]thiophen-2-yl)prop- |
| | 2-yn-1-yl)amino)-3-methoxybenzenesulfonamide; |
| 46 | 4-((3-(3-(cyclopropylmethyl)-7-(((3R,4R)-3-fluoro-1-methylpiperidin-4-yl)amino)benzo[b]thiophen-2-yl)prop- |
| | 2-yn-1-yl)amino)-3-methoxybenzenesulfonamide; |
| 47 | 4-((3-(3-(2,2-difluoroethyl)-7-(((3R,4R)-3-fluoro-1-methylpiperidin-4-yl)amino)benzo[b]thiophen-2-yl)prop- |
| | 2-yn-1-yl)amino)-3-methoxybenzenesulfonamide; |
| 48 | 4-((3-(7-(((3R,4R)-3-fluoro-1-methylpiperidin-4-yl)amino)-3-(1H-pyrrol-1-yl)benzo[b]thiophen-2-yl)prop- |
| | 2-yn-1-yl)amino)-3-methoxybenzenesulfonamide; |
| 49 | 4-((3-(3-ethyl-7-(((3R,4R)-3-fluoro-1-methylpiperidin-4-yl)amino)benzo[b]thiophen-2-yl)prop-2-yn-1-yl)amino)- |
| | 3-methoxybenzenesulfonamide; |
| 50 | 4-((3-(3-(cyanomethyl)-7-(((3S,4S)-3-fluoro-1-methylpiperidin-4-yl)amino)benzo[b]thiophen-2-yl)prop-2-yn- |
| | 1-yl)amino)-3-methoxybenzenesulfonamide; |
| 51 | 4-((3-(7-(((3S,4S)-3-fluoro-1-methylpiperidin-4-yl)amino)-3-(thiazol-4-yl)benzo[b]thiophen-2-yl)prop-2-yn- |
| | 1-yl)amino)-3-methoxybenzenesulfonamide; |
| 52 | 4-((3-(7-(((3S,4S)-3-fluoro-1-methylpiperidin-4-yl)amino)-3-(2,2,2-trifluoroethyl)benzo[b]thiophen-2-yl)prop- |
| | 2-yn-1-yl)amino)-3-methoxybenzenesulfonamide; |
| 53 | 4-((3-(3-(cyclopropylmethyl)-7-(((3S,4S)-3-fluoro-1-methylpiperidin-4-yl)amino)benzo[b]thiophen-2-yl)prop- |
| | 2-yn-1-yl)amino)-3-methoxybenzenesulfonamide; |
| 54 | 4-((3-(3-(2,2-difluoroethyl)-7-(((3S,4S)-3-fluoro-1-methylpiperidin-4-yl)amino)benzo[b]thiophen-2-yl)prop- |
| | 2-yn-1-yl)amino)-3-methoxybenzenesulfonamide; |
| 55 | 4-((3-(7-(((3S,4S)-3-fluoro-1-methylpiperidin-4-yl)amino)-3-(1H-pyrrol-1-yl)benzo[b]thiophen-2-yl)prop-2- |
| | yn-1-yl)amino)-3-methoxybenzenesulfonamide; |
| 56 | 4-((3-(3-ethyl-7-(((3S,4S)-3-fluoro-1-methylpiperidin-4-yl)amino)benzo[b]thiophen-2-yl)prop-2-yn-1-yl)amino)- |
| | 3-methoxybenzenesulfonamide; |
| 57 | 4-((3-(3-(cyanomethyl)-7-((4-(dimethylamino)cyclohexyl)amino)benzo[b]thiophen-2-yl)prop-2-yn-1-yl)amino)- |
| | 3-methoxybenzenesulfonamide; |
| 58 | 4-((3-(7-((4-(dimethylamino)cyclohexyl)amino)-3-(thiazol-4-yl)benzo[b]thiophen-2-yl)prop-2-yn-1-yl)amino)- |
| | 3-methoxybenzenesulfonamide; |
| 59 | 4-((3-(7-((4-(dimethylamino)cyclohexyl)amino)-3-(2,2,2-trifluoroethyl)benzo[b]thiophen-2-yl)prop-2-yn-1- |
| | yl)amino)-3-methoxybenzenesulfonamide; |
| 60 | 4-((3-(3-(cyclopropylmethyl)-7-((4-(dimethylamino)cyclohexyl)amino)benzo[b]thiophen-2-yl)prop-2-yn-1- |
| | yl)amino)-3-methoxybenzenesulfonamide; |
| 61 | 4-((3-(3-(2,2-difluoroethyl)-7-((4-(dimethylamino)cyclohexyl)amino)benzo[b]thiophen-2-yl)prop-2-yn-1- |
| | yl)amino)-3-methoxybenzenesulfonamide; |
| 62 | 4-((3-(7-((4-(dimethylamino)cyclohexyl)amino)-3-(1H-pyrrol-1-yl)benzo[b]thiophen-2-yl)prop-2-yn-1-yl)amino)- |
| | 3-methoxybenzenesulfonamide; |
| 63 | 4-((3-(7-((4-(dimethylamino)cyclohexyl)amino)-3-ethylbenzo[b]thiophen-2-yl)prop-2-yn-1-yl)amino)-3- |
| | methoxybenzenesulfonamide; |
| 64 | 4-((3-(3-(cyanomethyl)-7-((2-(dimethylamino)ethyl)amino)benzo[b]thiophen-2-yl)prop-2-yn-1-yl)amino)-3- |
| | methoxybenzenesulfonamide; |
| 65 | 4-((3-(7-((2-(dimethylamino)ethyl)amino)-3-(thiazol-4-yl)benzo[b]thiophen-2-yl)prop-2-yn-1-yl)amino)-3- |
| | methoxybenzenesulfonamide; |
| 66 | 4-((3-(7-((2-(dimethylamino)ethyl)amino)-3-(2,2,2-trifluoroethyl)benzo[b]thiophen-2-yl)prop-2-yn-1-yl)amino)- |
| | 3-methoxybenzenesulfonamide; |
| 67 | 4-((3-(3-(cyclopropylmethyl)-7-((2-(dimethylamino)ethyl)amino)benzo[b]thiophen-2-yl)prop-2-yn-1-yl)amino)- |
| | 3-methoxybenzenesulfonamide; |
| 68 | 4-((3-(3-(2,2-difluoroethyl)-7-((2-(dimethylamino)ethyl)amino)benzo[b]thiophen-2-yl)prop-2-yn-1-yl)amino)- |
| | 3-methoxybenzenesulfonamide; |
| 69 | 4-((3-(7-((2-(dimethylamino)ethyl)amino)-3-(1H-pyrrol-1-yl)benzo[b]thiophen-2-yl)prop-2-yn-1-yl)amino)- |
| | 3-methoxybenzenesulfonamide; |
| 70 | 4-((3-(7-((2-(dimethylamino)ethyl)amino)-3-ethylbenzo[b]thiophen-2-yl)prop-2-yn-1-yl)amino)-3- |
| | methoxybenzenesulfonamide; |
| 71 | 4-((3-(3-(cyanomethyl)-7-(((3S,4R)-3-fluoro-1-methylpiperidin-4-yl)amino)benzo[b]thiophen-2-yl)prop-2- |
| | yn-1-yl)amino)-3-methoxybenzenesulfonamide; |
| 72 | 4-((3-(7-(((3S,4R)-3-fluoro-1-methylpiperidin-4-yl)amino)-3-(thiazol-4-yl)benzo[b]thiophen-2-yl)prop-2-yn- |
| | 1-yl)amino)-3-methoxybenzenesulfonamide; |
| 73 | 4-((3-(7-(((3S,4R)-3-fluoro-1-methylpiperidin-4-yl)amino)-3-(2,2,2-trifluoroethyl)benzo[b]thiophen-2-yl)prop- |
| | 2-yn-1-yl)amino)-3-methoxybenzenesulfonamide; |
| 74 | 4-((3-(3-(cyclopropylmethyl)-7-(((3S,4R)-3-fluoro-1-methylpiperidin-4-yl)amino)benzo[b]thiophen-2-yl)prop- |
| | 2-yn-1-yl)amino)-3-methoxybenzenesulfonamide; |
| 75 | 4-((3-(3-(2,2-difluoroethyl)-7-(((3S,4R)-3-fluoro-1-methylpiperidin-4-yl)amino)benzo[b]thiophen-2-yl)prop- |
| | 2-yn-1-yl)amino)-3-methoxybenzenesulfonamide; |
| 76 | 4-((3-(7-(((3S,4R)-3-fluoro-1-methylpiperidin-4-yl)amino)-3-(1H-pyrrol-1-yl)benzo[b]thiophen-2-yl)prop-2- |
| | yn-1-yl)amino)-3-methoxybenzenesulfonamide; |
| 77 | 4-((3-(3-ethyl-7-(((3S,4R)-3-fluoro-1-methylpiperidin-4-yl)amino)benzo[b]thiophen-2-yl)prop-2-yn-1-yl)amino)- |
| | 3-methoxybenzenesulfonamide; |
| 78 | 4-((3-(3-(cyanomethyl)-7-((1-methylpiperidin-4-yl)amino)benzo[b]thiophen-2-yl)prop-2-yn-1-yl)amino)-3- |
| | methoxybenzenesulfonamide; |
| 79 | 3-methoxy-4-((3-(7-((1-methylpiperidin-4-yl)amino)-3-(thiazol-4-yl)benzo[b]thiophen-2-yl)prop-2-yn-1-yl) |
| | amino)benzenesulfonamide; |
| 80 | 3-methoxy-4-((3-(7-((1-methylpiperidin-4-yl)amino)-3-(2,2,2-trifluoroethyl)benzo[b]thiophen-2-yl)prop-2- |
| | yn-1-yl)amino)benzenesulfonamide; |
| 81 | 4-((3-(3-(cyclopropylmethyl)-7-((1-methylpiperidin-4-yl)amino)benzo[b]thiophen-2-yl)prop-2-yn-1-yl)amino)- |
| | 3-methoxybenzenesulfonamide; |
| 82 | 4-((3-(3-(2,2-difluoroethyl)-7-((1-methylpiperidin-4-yl)amino)benzo[b]thiophen-2-yl)prop-2-yn-1-yl)amino)- |
| | 3-methoxybenzenesulfonamide; |
| 83 | 3-methoxy-4-((3-(7-((1-methylpiperidin-4-yl)amino)-3-(1H-pyrrol-1-yl)benzo[b]thiophen-2-yl)prop-2-yn-1- |
| | yl)amino)benzenesulfonamide; |
| 84 | 4-((3-(3-ethyl-7-((1-methylpiperidin-4-yl)amino)benzo[b]thiophen-2-yl)prop-2-yn-1-yl)amino)-3- |
| | methoxybenzenesulfonamide; |
| 85 | 4-((3-(3-(cyanomethyl)-7-((tetrahydro-2H-pyran-4-yl)amino)benzo[b]thiophen-2-yl)prop-2-yn-1-yl)amino)- |
| | 3-methoxybenzenesulfonamide; |
| 86 | 3-methoxy-4-((3-(7-((tetrahydro-2H-pyran-4-yl)amino)-3-(thiazol-4-yl)benzo[b]thiophen-2-yl)prop-2-yn-1- |
| | yl)amino)benzenesulfonamide; |
| 87 | 3-methoxy-4-((3-(7-((tetrahydro-2H-pyran-4-yl)amino)-3-(2,2,2-trifluoroethyl)benzo[b]thiophen-2-yl)prop- |
| | 2-yn-1-yl)amino)benzenesulfonamide; |
| 88 | 4-((3-(3-(cyclopropylmethyl)-7-((tetrahydro-2H-pyran-4-yl)amino)benzo[b]thiophen-2-yl)prop-2-yn-1-yl)amino)- |
| | 3-methoxybenzenesulfonamide; |
| 89 | 4-((3-(3-(2,2-difluoroethyl)-7-((tetrahydro-2H-pyran-4-yl)amino)benzo[b]thiophen-2-yl)prop-2-yn-1-yl)amino)- |
| | 3-methoxybenzenesulfonamide; |
| 90 | 4-((3-(3-(1H-pyrrol-1-yl)-7-((tetrahydro-2H-pyran-4-yl)amino)benzo[b]thiophen-2-yl)prop-2-yn-1-yl)amino)- |
| | 3-methoxybenzenesulfonamide; |
| 91 | 4-((3-(3-ethyl-7-((tetrahydro-2H-pyran-4-yl)amino)benzo[b]thiophen-2-yl)prop-2-yn-1-yl)amino)-3- |
| | methoxybenzenesulfonamide; |
| 92 | 2-(7-((3,3-difluoro-1-methylpiperidin-4-yl)amino)-2-(3-((5-fluoro-2-methoxy-4-(methylsulfonyl)phenyl)amino)prop- |
| | 1-yn-1-yl)benzo[b]thiophen-3-yl)acetonitrile; |
| 93 | 3,3-difluoro-N-(2-(3-((5-fluoro-2-methoxy-4-(methylsulfonyl)phenyl)amino)prop-1-yn-1-yl)-3-(thiazol-4- |
| | yl)benzo[b]thiophen-7-yl)-1-methylpiperidin-4-amine; |
| 94 | 3,3-difluoro-N-(2-(3-((5-fluoro-2-methoxy-4-(methylsulfonyl)phenyl)amino)prop-1-yn-1-yl)-3-(2,2,2- |
| | trifluoroethyl)benzo[b]thiophen-7-yl)-1-methylpiperidin-4-amine; |
| 95 | N-(3-(cyclopropylmethyl)-2-(3-((5-fluoro-2-methoxy-4-(methylsulfonyl)phenyl)amino)prop-1-yn-1- |
| | yl)benzo[b]thiophen-7-yl)-3,3-difluoro-1-methylpiperidin-4-amine; |
| 96 | N-(3-(2,2-difluoroethyl)-2-(3-((5-fluoro-2-methoxy-4-(methylsulfonyl)phenyl)amino)prop-1-yn-1- |
| | yl)benzo[b]thiophen-7-yl)-3,3-difluoro-1-methylpiperidin-4-amine; |
| 97 | 3,3-difluoro-N-(2-(3-((5-fluoro-2-methoxy-4-(methylsulfonyl)phenyl)amino)prop-1-yn-1-yl)-3-(1H-pyrrol- |
| | 1-yl)benzo[b]thiophen-7-yl)-1-methylpiperidin-4-amine; |
| 98 | N-(3-ethyl-2-(3-((5-fluoro-2-methoxy-4-(methylsulfonyl)phenyl)amino)prop-1-yn-1-yl)benzo[b]thiophen-7- |
| | yl)-3,3-difluoro-1-methylpiperidin-4-amine; |
| 99 | 2-(7-((1,1-dioxidotetrahydro-2H-thiopyran-4-yl)amino)-2-(3-((5-fluoro-2-methoxy-4- |
| | (methylsulfonyl)phenyl)amino)prop-1-yn-1-yl)benzo[b]thiophen-3-yl)acetonitrile; |
| 100 | 4-((2-(3-((5-fluoro-2-methoxy-4-(methylsulfonyl)phenyl)amino)prop-1-yn-1-yl)-3-(thiazol-4-yl)benzo[b]thiophen- |
| | 7-yl)amino)tetrahydro-2H-thiopyran 1,1-dioxide; |
| 101 | 4-((2-(3-((5-fluoro-2-methoxy-4-(methylsulfonyl)phenyl)amino)prop-1-yn-1-yl)-3-(2,2,2-trifluoroethyl)benzo[b]thiophen- |
| | 7-yl)amino)tetrahydro-2H-thiopyran 1,1-dioxide; |
| 102 | 4-((3-(cyclopropylmethyl)-2-(3-((5-fluoro-2-methoxy-4-(methylsulfonyl)phenyl)amino)prop-1-yn-1-yl)benzo[b]thiophen- |
| | 7-yl)amino)tetrahydro-2H-thiopyran 1,1-dioxide; |
| 103 | 4-((3-(2,2-difluoroethyl)-2-(3-((5-fluoro-2-methoxy-4-(methylsulfonyl)phenyl)amino)prop-1-yn-1- |
| | yl)benzo[b]thiophen-7-yl)amino)tetrahydro-2H-thiopyran 1,1-dioxide; |
| 104 | 4-((2-(3-((5-fluoro-2-methoxy-4-(methylsulfonyl)phenyl)amino)prop-1-yn-1-yl)-3-(1H-pyrrol-1- |
| | yl)benzo[b]thiophen-7-yl)amino)tetrahydro-2H-thiopyran 1,1-dioxide; |
| 105 | 4-((3-ethyl-2-(3-((5-fluoro-2-methoxy-4-(methylsulfonyl)phenyl)amino)prop-1-yn-1-yl)benzo[b]thiophen-7- |
| | yl)amino)tetrahydro-2H-thiopyran 1,1-dioxide; |
| 106 | 2-(7-(((3R,4S)-3-fluoro-1-methylpiperidin-4-yl)amino)-2-(3-((5-fluoro-2-methoxy-4-(methylsulfonyl)phenyl)amino)prop- |
| | 1-yn-1-yl)benzo[b]thiophen-3-yl)acetonitrile; |
| 107 | (3R,4S)-3-fluoro-N-(2-(3-((5-fluoro-2-methoxy-4-(methylsulfonyl)phenyl)amino)prop-1-yn-1-yl)-3-(thiazol- |
| | 4-yl)benzo[b]thiophen-7-yl)-1-methylpiperidin-4-amine; |
| 108 | (3R,4S)-3-fluoro-N-(2-(3-((5-fluoro-2-methoxy-4-(methylsulfonyl)phenyl)amino)prop-1-yn-1-yl)-3-(2,2,2- |
| | trifluoroethyl)benzo[b]thiophen-7-yl)-1-methylpiperidin-4-amine; |
| 109 | (3R,4S)-N-(3-(cyclopropylmethyl)-2-(3-((5-fluoro-2-methoxy-4-(methylsulfonyl)phenyl)amino)prop-1-yn- |
| | 1-yl)benzo[b]thiophen-7-yl)-3-fluoro-1-methylpiperidin-4-amine; |
| 110 | (3R,4S)-N-(3-(2,2-difluoroethyl)-2-(3-((5-fluoro-2-methoxy-4-(methylsulfonyl)phenyl)amino)prop-1-yn-1- |
| | yl)benzo[b]thiophen-7-yl)-3-fluoro-1-methylpiperidin-4-amine; |
| 111 | (3R,4S)-3-fluoro-N-(2-(3-((5-fluoro-2-methoxy-4-(methylsulfonyl)phenyl)amino)prop-1-yn-1-yl)-3-(1H-pyrrol- |
| | 1-yl)benzo[b]thiophen-7-yl)-1-methylpiperidin-4-amine; |
| 112 | (3R,4S)-N-(3-ethyl-2-(3-((5-fluoro-2-methoxy-4-(methylsulfonyl)phenyl)amino)prop-1-yn-1-yl)benzo[b]thiophen- |
| | 7-yl)-3-fluoro-1-methylpiperidin-4-amine; |
| 113 | 2-(7-((3-fluoro-1-methylpiperidin-4-yl)amino)-2-(3-((5-fluoro-2-methoxy-4-(methylsulfonyl)phenyl)amino)prop- |
| | 1-yn-1-yl)benzo[b]thiophen-3-yl)acetonitrile; |
| 114 | 3-fluoro-N-(2-(3-((5-fluoro-2-methoxy-4-(methylsulfonyl)phenyl)amino)prop-1-yn-1-yl)-3-(thiazol-4- |
| | yl)benzo[b]thiophen-7-yl)-1-methylpiperidin-4-amine; |
| 115 | 3-fluoro-N-(2-(3-((5-fluoro-2-methoxy-4-(methylsulfonyl)phenyl)amino)prop-1-yn-1-yl)-3-(2,2,2- |
| | trifluoroethyl)benzo[b]thiophen-7-yl)-1-methylpiperidin-4-amine; |
| 116 | N-(3-(cyclopropylmethyl)-2-(3-((5-fluoro-2-methoxy-4-(methylsulfonyl)phenyl)amino)prop-1-yn-1- |
| | yl)benzo[b]thiophen-7-yl)-3-fluoro-1-methylpiperidin-4-amine; |
| 117 | N-(3-(2,2-difluoroethyl)-2-(3-((5-fluoro-2-methoxy-4-(methylsulfonyl)phenyl)amino)prop-1-yn-1- |
| | yl)benzo[b]thiophen-7-yl)-3-fluoro-1-methylpiperidin-4-amine; |
| 118 | 3-fluoro-N-(2-(3-((5-fluoro-2-methoxy-4-(methylsulfonyl)phenyl)amino)prop-1-yn-1-yl)-3-(1H-pyrrol-1- |
| | yl)benzo[b]thiophen-7-yl)-1-methylpiperidin-4-amine; |
| 119 | N-(3-ethyl-2-(3-((5-fluoro-2-methoxy-4-(methylsulfonyl)phenyl)amino)prop-1-yn-1-yl)benzo[b]thiophen-7- |
| | yl)-3-fluoro-1-methylpiperidin-4-amine; |
| 120 | 2-(7-((1-(dimethylglycyl)piperidin-4-yl)amino)-2-(3-((5-fluoro-2-methoxy-4-(methylsulfonyl)phenyl)amino)prop- |
| | 1-yn-1-yl)benzo[b]thiophen-3-yl)acetonitrile; |
| 121 | 2-(dimethylamino)-1-(4-((2-(3-((5-fluoro-2-methoxy-4-(methylsulfonyl)phenyl)amino)prop-1-yn-1-yl)-3- |
| | (thiazol-4-yl)benzo[b]thiophen-7-yl)amino)piperidin-1-yl)ethan-1-one; |
| 122 | 2-(dimethylamino)-1-(4-((2-(3-((5-fluoro-2-methoxy-4-(methylsulfonyl)phenyl)amino)prop-1-yn-1-yl)-3- |
| | (2,2,2-trifluoroethyl)benzo[b]thiophen-7-yl)amino)piperidin-1-yl)ethan-1-one; |
| 123 | 1-(4-((3-(cyclopropylmethyl)-2-(3-((5-fluoro-2-methoxy-4-(methylsulfonyl)phenyl)amino)prop-1-yn-1- |
| | yl)benzo[b]thiophen-7-yl)amino)piperidin-1-yl)-2-(dimethylamino)ethan-1-one; |
| 124 | 1-(4-((3-(2,2-difluoroethyl)-2-(3-((5-fluoro-2-methoxy-4-(methylsulfonyl)phenyl)amino)prop-1-yn-1- |
| | yl)benzo[b]thiophen-7-yl)amino)piperidin-1-yl)-2-(dimethylamino)ethan-1-one; |
| 125 | 2-(dimethylamino)-1-(4-((2-(3-((5-fluoro-2-methoxy-4-(methylsulfonyl)phenyl)amino)prop-1-yn-1-yl)-3- |
| | (1H-pyrrol-1-yl)benzo[b]thiophen-7-yl)amino)piperidin-1-yl)ethan-1-one; |
| 126 | 2-(dimethylamino)-1-(4-((3-ethyl-2-(3-((5-fluoro-2-methoxy-4-(methylsulfonyl)phenyl)amino)prop-1-yn-1- |
| | yl)benzo[b]thiophen-7-yl)amino)piperidin-1-yl)ethan-1-one; |
| 127 | 2-(7-((3-fluoro-1-(2-hydroxy-3-methoxypropyl)piperidin-4-yl)amino)-2-(3-((5-fluoro-2-methoxy-4- |
| | (methylsulfonyl)phenyl)amino)prop-1-yn-1-yl)benzo[b]thiophen-3-yl)acetonitrile; |
| 128 | 1-(3-fluoro-4-((2-(3-((5-fluoro-2-methoxy-4-(methylsulfonyl)phenyl)amino)prop-1-yn-1-yl)-3-(thiazol-4- |
| | yl)benzo[b]thiophen-7-yl)amino)piperidin-1-yl)-3-methoxypropan-2-ol; |
| 129 | 1-(3-fluoro-4-((2-(3-((5-fluoro-2-methoxy-4-(methylsulfonyl)phenyl)amino)prop-1-yn-1-yl)-3-(2,2,2- |
| | trifluoroethyl)benzo[b]thiophen-7-yl)amino)piperidin-1-yl)-3-methoxypropan-2-ol; |
| 130 | 1-(4-((3-(cyclopropylmethyl)-2-(3-((5-fluoro-2-methoxy-4-(methylsulfonyl)phenyl)amino)prop-1-yn-1- |
| | yl)benzo[b]thiophen-7-yl)amino)-3-fluoropiperidin-1-yl)-3-methoxypropan-2-o1; |
| 131 | 1-(4-((3-(2,2-difluoroethyl)-2-(3-((5-fluoro-2-methoxy-4-(methylsulfonyl)phenyl)amino)prop-1-yn-1- |
| | yl)benzo[b]thiophen-7-yl)amino)-3-fluoropiperidin-1-yl)-3-methoxypropan-2-ol; |
| 132 | 1-(3-fluoro-4-((2-(3-((5-fluoro-2-methoxy-4-(methylsulfonyl)phenyl)amino)prop-1-yn-1-yl)-3-(1H-pyrrol- |
| | 1-yl)benzo[b]thiophen-7-yl)amino)piperidin-1-yl)-3-methoxypropan-2-ol; |
| 133 | 1-(4-((3-ethyl-2-(3-((5-fluoro-2-methoxy-4-(methylsulfonyl)phenyl)amino)prop-1-yn-1-yl)benzo[b]thiophen- |
| | 7-yl)amino)-3-fluoropiperidin-1-yl)-3-methoxypropan-2-ol; |
| 134 | 2-(7-(((3R,4R)-3-fluoro-1-methylpiperidin-4-yl)amino)-2-(3-((5-fluoro-2-methoxy-4-(methylsulfonyl)phenyl)amino)prop- |
| | 1-yn-1-yl)benzo[b]thiophen-3-yl)acetonitrile; |
| 135 | (3R,4R)-3-fluoro-N-(2-(3-((5-fluoro-2-methoxy-4-(methylsulfonyl)phenyl)amino)prop-1-yn-1-yl)-3-(thiazol- |
| | 4-yl)benzo[b]thiophen-7-yl)-1-methylpiperidin-4-amine; |
| 136 | (3R,4R)-3-fluoro-N-(2-(3-((5-fluoro-2-methoxy-4-(methylsulfonyl)phenyl)amino)prop-1-yn-1-yl)-3-(2,2,2- |
| | trifluoroethyl)benzo[b]thiophen-7-yl)-1-methylpiperidin-4-amine; |
| 137 | (3R,4R)-N-(3-(cyclopropylmethyl)-2-(3-((5-fluoro-2-methoxy-4-(methylsulfonyl)phenyl)amino)prop-1-yn- |
| | 1-yl)benzo[b]thiophen-7-yl)-3-fluoro-1-methylpiperidin-4-amine; |
| 138 | (3R,4R)-N-(3-(2,2-difluoroethyl)-2-(3-((5-fluoro-2-methoxy-4-(methylsulfonyl)phenyl)amino)prop-1-yn-1- |
| | yl)benzo[b]thiophen-7-yl)-3-fluoro-1-methylpiperidin-4-amine; |
| 139 | (3R,4R)-3-fluoro-N-(2-(3-((5-fluoro-2-methoxy-4-(methylsulfonyl)phenyl)amino)prop-1-yn-1-yl)-3-(1H- |
| | pyrrol-1-yl)benzo[b]thiophen-7-yl)-1-methylpiperidin-4-amine; |
| 140 | (3R,4R)-N-(3-ethyl-2-(3-((5-fluoro-2-methoxy-4-(methylsulfonyl)phenyl)amino)prop-1-yn-1-yl)benzo[b]thiophen- |
| | 7-yl)-3-fluoro-1-methylpiperidin-4-amine; |
| 141 | 2-(7-(((3S,4S)-3-fluoro-1-methylpiperidin-4-yl)amino)-2-(3-((5-fluoro-2-methoxy-4-(methylsulfonyl)phenyl)amino)prop- |
| | 1-yn-1-yl)benzo[b]thiophen-3-yl)acetonitrile; |
| 142 | (3S,4S)-3-fluoro-N-(2-(3-((5-fluoro-2-methoxy-4-(methylsulfonyl)phenyl)amino)prop-1-yn-1-yl)-3-(thiazol- |
| | 4-yl)benzo[b]thiophen-7-yl)-1-methylpiperidin-4-amine; |
| 143 | (3S,4S)-3-fluoro-N-(2-(3-((5-fluoro-2-methoxy-4-(methylsulfonyl)phenyl)amino)prop-1-yn-1-yl)-3-(2,2,2- |
| | trifluoroethyl)benzo[b]thiophen-7-yl)-1-methylpiperidin-4-amine; |
| 144 | (3S,4S)-N-(3-(cyclopropylmethyl)-2-(3-((5-fluoro-2-methoxy-4-(methylsulfonyl)phenyl)amino)prop-1-yn-1- |
| | yl)benzo[b]thiophen-7-yl)-3-fluoro-1-methylpiperidin-4-amine; |
| 145 | (3S,4S)-N-(3-(2,2-difluoroethyl)-2-(3-((5-fluoro-2-methoxy-4-(methylsulfonyl)phenyl)amino)prop-1-yn-1- |
| | yl)benzo[b]thiophen-7-yl)-3-fluoro-1-methylpiperidin-4-amine; |
| 146 | (3S,4S)-3-fluoro-N-(2-(3-((5-fluoro-2-methoxy-4-(methylsulfonyl)phenyl)amino)prop-1-yn-1-yl)-3-(1H-pyrrol- |
| | 1-yl)benzo[b]thiophen-7-yl)-1-methylpiperidin-4-amine; |
| 147 | (3S,4S)-N-(3-ethyl-2-(3-((5-fluoro-2-methoxy-4-(methylsulfonyl)phenyl)amino)prop-1-yn-1-yl)benzo[b]thiophen- |
| | 7-yl)-3-fluoro-1-methylpiperidin-4-amine; |
| 148 | 2-(7-((4-(dimethylamino)cyclohexyl)amino)-2-(3-((5-fluoro-2-methoxy-4-(methylsulfonyl)phenyl)amino)prop- |
| | 1-yn-1-yl)benzo[b]thiophen-3-yl)acetonitrile; |
| 149 | N1-(2-(3-((5-fluoro-2-methoxy-4-(methylsulfonyl)phenyl)amino)prop-1-yn-1-yl)-3-(thiazol-4-yl)benzo[b]thiophen- |
| | 7-yl)-N4,N4-dimethylcyclohexane-1,4-diamine; |
| 150 | N1-(2-(3-((5-fluoro-2-methoxy-4-(methylsulfonyl)phenyl)amino)prop-1-yn-1-yl)-3-(2,2,2-trifluoroethyl)benzo[b]thiophen- |
| | 7-yl)-N4,N4-dimethylcyclohexane-1,4-diamine; |
| 151 | N1-(3-(cyclopropylmethyl)-2-(3-((5-fluoro-2-methoxy-4-(methylsulfonyl)phenyl)amino)prop-1-yn-1- |
| | yl)benzo[b]thiophen-7-yl)-N4,N4-dimethylcyclohexane-1,4-diamine; |
| 152 | N1-(3-(2,2-difluoroethyl)-2-(3-((5-fluoro-2-methoxy-4-(methylsulfonyl)phenyl)amino)prop-1-yn-1- |
| | yl)benzo[b]thiophen-7-yl)-N4,N4-dimethylcyclohexane-1,4-diamine; |
| 153 | N1-(2-(3-((5-fluoro-2-methoxy-4-(methylsulfonyl)phenyl)amino)prop-1-yn-1-yl)-3-(1H-pyrrol-1- |
| | yl)benzo[b]thiophen-7-yl)-N4,N4-dimethylcyclohexane-1,4-diamine; |
| 154 | N1-(3-ethyl-2-(3-((5-fluoro-2-methoxy-4-(methylsulfonyl)phenyl)amino)prop-1-yn-1-yl)benzo[b]thiophen- |
| | 7-yl)-N4,N4-dimethylcyclohexane-1,4-diamine; |
| 155 | 2-(7-((2-(dimethylamino)ethyl)amino)-2-(3-((5-fluoro-2-methoxy-4-(methylsulfonyl)phenyl)amino)prop-1- |
| | yn-1-yl)benzo[b]thiophen-3-yl)acetonitrile; |
| 156 | N1-(2-(3-((5-fluoro-2-methoxy-4-(methylsulfonyl)phenyl)amino)prop-1-yn-1-yl)-3-(thiazol-4- |
| | yl)benzo[b]thiophen-7-yl)-N2,N2-dimethylethane-1,2-diamine; |
| 157 | N1-(2-(3-((5-fluoro-2-methoxy-4-(methylsulfonyl)phenyl)amino)prop-1-yn-1-yl)-3-(2,2,2- |
| | trifluoroethyl)benzo[b]thiophen-7-yl)-N2,N2-dimethylethane-1,2-diamine; |
| 158 | N1-(3-(cyclopropylmethyl)-2-(3-((5-fluoro-2-methoxy-4-(methylsulfonyl)phenyl)amino)prop-1-yn-1- |
| | yl)benzo[b]thiophen-7-yl)-N2,N2-dimethylethane-1,2-diamine; |
| 159 | N1-(3-(2,2-difluoroethyl)-2-(3-((5-fluoro-2-methoxy-4-(methylsulfonyl)phenyl)amino)prop-1-yn-1- |
| | yl)benzo[b]thiophen-7-yl)-N2,N2-dimethylethane-1,2-diamine; |
| 160 | N1-(2-(3-((5-fluoro-2-methoxy-4-(methylsulfonyl)phenyl)amino)prop-1-yn-1-yl)-3-(1H-pyrrol-1- |
| | yl)benzo[b]thiophen-7-yl)-N2,N2-dimethylethane-1,2-diamine; |
| 161 | N1-(3-ethyl-2-(3-((5-fluoro-2-methoxy-4-(methylsulfonyl)phenyl)amino)prop-1-yn-1-yl)benzo[b]thiophen- |
| | 7-yl)-N2,N2-dimethylethane-1,2-diamine; |
| 162 | 2-(7-(((3S,4R)-3-fluoro-1-methylpiperidin-4-yl)amino)-2-(3-((5-fluoro-2-methoxy-4-(methylsulfonyl)phenyl)amino)prop- |
| | 1-yn-1-yl)benzo[b]thiophen-3-yl)acetonitrile; |
| 163 | (3S,4R)-3-fluoro-N-(2-(3-((5-fluoro-2-methoxy-4-(methylsulfonyl)phenyl)amino)prop-1-yn-1-yl)-3-(thiazol- |
| | 4-yl)benzo[b]thiophen-7-yl)-1-methylpiperidin-4-amine; |
| 164 | (3S,4R)-3-fluoro-N-(2-(3-((5-fluoro-2-methoxy-4-(methylsulfonyl)phenyl)amino)prop-1-yn-1-yl)-3-(2,2,2- |
| | trifluoroethyl)benzo[b]thiophen-7-yl)-1-methylpiperidin-4-amine; |
| 165 | (3S,4R)-N-(3-(cyclopropylmethyl)-2-(3-((5-fluoro-2-methoxy-4-(methylsulfonyl)phenyl)amino)prop-1-yn- |
| | 1-yl)benzo[b]thiophen-7-yl)-3-fluoro-1-methylpiperidin-4-amine; |
| 166 | (3S,4R)-N-(3-(2,2-difluoroethyl)-2-(3-((5-fluoro-2-methoxy-4-(methylsulfonyl)phenyl)amino)prop-1-yn-1- |
| | yl)benzo[b]thiophen-7-yl)-3-fluoro-1-methylpiperidin-4-amine; |
| 167 | (3S,4R)-3-fluoro-N-(2-(3-((5-fluoro-2-methoxy-4-(methylsulfonyl)phenyl)amino)prop-1-yn-1-yl)-3-(1H- |
| | pyrrol-1-yl)benzo[b]thiophen-7-yl)-1-methylpiperidin-4-amine; |
| 168 | (3S,4R)-N-(3-ethyl-2-(3-((5-fluoro-2-methoxy-4-(methylsulfonyl)phenyl)amino)prop-1-yn-1-yl)benzo[b]thiophen- |
| | 7-yl)-3-fluoro-1-methylpiperidin-4-amine; |
| 169 | 2-(2-(3-((5-fluoro-2-methoxy-4-(methylsulfonyl)phenyl)amino)prop-1-yn-1-yl)-7-((1-methylpiperidin-4-yl) |
| | amino)benzo[b]thiophen-3-yl)acetonitrile; |
| 170 | N-(2-(3-((5-fluoro-2-methoxy-4-(methylsulfonyl)phenyl)amino)prop-1-yn-1-yl)-3-(thiazol-4-yl)benzo[b]thiophen- |
| | 7-yl)-1-methylpiperidin-4-amine; |
| 171 | N-(2-(3-((5-fluoro-2-methoxy-4-(methylsulfonyl)phenyl)amino)prop-1-yn-1-yl)-3-(2,2,2-trifluoroethyl)benzo[b]thiophen- |
| | 7-yl)-1-methylpiperidin-4-amine; |
| 172 | N-(3-(cyclopropylmethyl)-2-(3-((5-fluoro-2-methoxy-4-(methylsulfonyl)phenyl)amino)prop-1-yn-1- |
| | yl)benzo[b]thiophen-7-yl)-1-methylpiperidin-4-amine; |
| 173 | N-(3-(2,2-difluoroethyl)-2-(3-((5-fluoro-2-methoxy-4-(methylsulfonyl)phenyl)amino)prop-1-yn-1-yl)benzo |
| | [b]thiophen-7-yl)-1-methylpiperidin-4-amine; |
| 174 | N-(2-(3-((5-fluoro-2-methoxy-4-(methylsulfonyl)phenyl)amino)prop-1-yn-1-yl)-3-(1H-pyrrol-1-yl)benzo[b] |
| | thiophen-7-yl)-1-methylpiperidin-4-amine; |
| 175 | N-(3-ethyl-2-(3-((5-fluoro-2-methoxy-4-(methylsulfonyl)phenyl)amino)prop-1-yn-1-yl)benzo[b]thiophen-7- |
| | yl)-1-methylpiperidin-4-amine; |
| 176 | 2-(2-(3-((5-fluoro-2-methoxy-4-(methylsulfonyl)phenyl)amino)prop-1-yn-1-yl)-7-((tetrahydro-2H-pyran-4- |
| | yl)amino)benzo[b]thiophen-3-yl)acetonitrile; |
| 177 | N-(2-(3-((5-fluoro-2-methoxy-4-(methylsulfonyl)phenyl)amino)prop-1-yn-1-yl)-3-(thiazol-4-yl)benzo[b]thiophen- |
| | 7-yl)tetrahydro-2H-pyran-4-amine; |
| 178 | N-(2-(3-((5-fluoro-2-methoxy-4-(methylsulfonyl)phenyl)amino)prop-1-yn-1-yl)-3-(2,2,2-trifluoroethyl) |
| | benzo[b]thiophen-7-yl)tetrahydro-2H-pyran-4-amine; |
| 179 | N-(3-(cyclopropylmethyl)-2-(3-((5-fluoro-2-methoxy-4-(methylsulfonyl)phenyl)amino)prop-1-yn-1-yl)benzo |
| | [b]thiophen-7-yl)tetrahydro-2H-pyran-4-amine; |
| 180 | N-(3-(2,2-difluoroethyl)-2-(3-((5-fluoro-2-methoxy-4-(methylsulfonyl)phenyl)amino)prop-1-yn-1-yl)benzo |
| | [b]thiophen-7-yl)tetrahydro-2H-pyran-4-amine; |
| 181 | N-(2-(3-((5-fluoro-2-methoxy-4-(methylsulfonyl)phenyl)amino)prop-1-yn-1-yl)-3-(1H-pyrrol-1-yl)benzo[b] |
| | thiophen-7-yl)tetrahydro-2H-pyran-4-amine; |
| 182 | N-(3-ethyl-2-(3-((5-fluoro-2-methoxy-4-(methylsulfonyl)phenyl)amino)prop-1-yn-1-yl)benzo[b]thiophen-7- |
| | yl)tetrahydro-2H-pyran-4-amine; |
| 183 | 4-((3-(3-(cyanomethyl)-7-((3,3-difluoro-1-methylpiperidin-4-yl)amino)benzo[b]thiophen-2-yl)prop-2-yn-1- |
| | yl)amino)-3-methoxy-N-methylbenzamide; |
| 184 | 4-((3-(7-((3,3-difluoro-1-methylpiperidin-4-yl)amino)-3-(thiazol-4-yl)benzo[b]thiophen-2-yl)prop-2-yn-1- |
| | yl)amino)-3-methoxy-N-methylbenzamide; |
| 185 | 4-((3-(7-((3,3-difluoro-1-methylpiperidin-4-yl)amino)-3-(2,2,2-trifluoroethyl)benzo[b]thiophen-2-yl)prop-2- |
| | yn-1-yl)amino)-3-methoxy-N-methylbenzamide; |
| 186 | 4-((3-(3-(cyclopropylmethyl)-7-((3,3-difluoro-1-methylpiperidin-4-yl)amino)benzo[b]thiophen-2-yl)prop-2- |
| | yn-1-yl)amino)-3-methoxy-N-methylbenzamide; |
| 187 | 4-((3-(7-((3,3-difluoro-1-methylpiperidin-4-yl)amino)-3-(2,2-difluoroethyl)benzo[b]thiophen-2-yl)prop-2- |
| | yn-1-yl)amino)-3-methoxy-N-methylbenzamide; |
| 188 | 4-((3-(7-((3,3-difluoro-1-methylpiperidin-4-yl)amino)-3-(1H-pyrrol-1-yl)benzo[b]thiophen-2-yl)prop-2-yn- |
| | 1-yl)amino)-3-methoxy-N-methylbenzamide; |
| 189 | 4-((3-(7-((3,3-difluoro-1-methylpiperidin-4-yl)amino)-3-ethylbenzo[b]thiophen-2-yl)prop-2-yn-1-yl)amino)- |
| | 3-methoxy-N-methylbenzamide; |
| 190 | 4-((3-(3-(cyanomethyl)-7-((1,1-dioxidotetrahydro-2H-thiopyran-4-yl)amino)benzo[b]thiophen-2-yl)prop-2- |
| | yn-1-yl)amino)-3-methoxy-N-methylbenzamide; |
| 191 | 4-((3-(7-((1,1-dioxidotetrahydro-2H-thiopyran-4-yl)amino)-3-(thiazol-4-yl)benzo[b]thiophen-2-yl)prop-2- |
| | yn-1-yl)amino)-3-methoxy-N-methylbenzamide; |
| 192 | 4-((3-(7-((1,1-dioxidotetrahydro-2H-thiopyran-4-yl)amino)-3-(2,2,2-trifluoroethyl)benzo[b]thiophen-2-yl)prop- |
| | 2-yn-1-yl)amino)-3-methoxy-N-methylbenzamide; |
| 193 | 4-((3-(3-(cyclopropylmethyl)-7-((1,1-dioxidotetrahydro-2H-thiopyran-4-yl)amino)benzo[b]thiophen-2-yl)prop- |
| | 2-yn-1-yl)amino)-3-methoxy-N-methylbenzamide; |
| 194 | 4-((3-(3-(2,2-difluoroethyl)-7-((1,1-dioxidotetrahydro-2H-thiopyran-4-yl)amino)benzo[b]thiophen-2-yl)prop- |
| | 2-yn-1-yl)amino)-3-methoxy-N-methylbenzamide; |
| 195 | 4-((3-(7-((1,1-dioxidotetrahydro-2H-thiopyran-4-yl)amino)-3-(1H-pyrrol-1-yl)benzo[b]thiophen-2-yl)prop- |
| | 2-yn-1-yl)amino)-3-methoxy-N-methylbenzamide; |
| 196 | 4-((3-(7-((1,1-dioxidotetrahydro-2H-thiopyran-4-yl)amino)-3-ethylbenzo[b]thiophen-2-yl)prop-2-yn-1-yl)amino)- |
| | 3-methoxy-N-methylbenzamide; |
| 197 | 4-((3-(3-(cyanomethyl)-7-(((3R,4S)-3-fluoro-1-methylpiperidin-4-yl)amino)benzo[b]thiophen-2-yl)prop-2- |
| | yn-1-yl)amino)-3-methoxy-N-methylbenzamide; |
| 198 | 4-((3-(7-(((3R,4S)-3-fluoro-1-methylpiperidin-4-yl)amino)-3-(thiazol-4-yl)benzo[b]thiophen-2-yl)prop-2- |
| | yn-1-yl)amino)-3-methoxy-N-methylbenzamide; |
| 199 | 4-((3-(7-(((3R,4S)-3-fluoro-1-methylpiperidin-4-yl)amino)-3-(2,2,2-trifluoroethyl)benzo[b]thiophen-2-yl)prop- |
| | 2-yn-1-yl)amino)-3-methoxy-N-methylbenzamide; |
| 200 | 4-((3-(3-(cyclopropylmethyl)-7-(((3R,4S)-3-fluoro-1-methylpiperidin-4-yl)amino)benzo[b]thiophen-2-yl)prop- |
| | 2-yn-1-yl)amino)-3-methoxy-N-methylbenzamide; |
| 201 | 4-((3-(3-(2,2-difluoroethyl)-7-(((3R,4S)-3-fluoro-1-methylpiperidin-4-yl)amino)benzo[b]thiophen-2-yl)prop- |
| | 2-yn-1-yl)amino)-3-methoxy-N-methylbenzamide; |
| 202 | 4-((3-(7-(((3R,4S)-3-fluoro-1-methylpiperidin-4-yl)amino)-3-(1H-pyrrol-1-yl)benzo[b]thiophen-2-yl)prop-2- |
| | yn-1-yl)amino)-3-methoxy-N-methylbenzamide; |
| 203 | 4-((3-(3-ethyl-7-(((3R,4S)-3-fluoro-1-methylpiperidin-4-yl)amino)benzo[b]thiophen-2-yl)prop-2-yn-1-yl)amino)- |
| | 3-methoxy-N-methylbenzamide; |
| 204 | 4-((3-(3-(cyanomethyl)-7-((3-fluoro-1-methylpiperidin-4-yl)amino)benzo[b]thiophen-2-yl)prop-2-yn-1-yl)amino)- |
| | 3-methoxy-N-methylbenzamide; |
| 205 | 4-((3-(7-((3-fluoro-1-methylpiperidin-4-yl)amino)-3-(thiazol-4-yl)benzo[b]thiophen-2-yl)prop-2-yn-1-yl)amino)- |
| | 3-methoxy-N-methylbenzamide; |
| 206 | 4-((3-(7-((3-fluoro-1-methylpiperidin-4-yl)amino)-3-(2,2,2-trifluoroethyl)benzo[b]thiophen-2-yl)prop-2-yn- |
| | 1-yl)amino)-3-methoxy-N-methylbenzamide; |
| 207 | 4-((3-(3-(cyclopropylmethyl)-7-((3-fluoro-1-methylpiperidin-4-yl)amino)benzo[b]thiophen-2-yl)prop-2-yn- |
| | 1-yl)amino)-3-methoxy-N-methylbenzamide; |
| 208 | 4-((3-(3-(2,2-difluoroethyl)-7-((3-fluoro-1-methylpiperidin-4-yl)amino)benzo[b]thiophen-2-yl)prop-2-yn-1- |
| | yl)amino)-3-methoxy-N-methylbenzamide; |
| 209 | 4-((3-(7-((3-fluoro-1-methylpiperidin-4-yl)amino)-3-(1H-pyrrol-1-yl)benzo[b]thiophen-2-yl)prop-2-yn-1-yl) |
| | amino)-3-methoxy-N-methylbenzamide; |
| 210 | 4-((3-(3-ethyl-7-((3-fluoro-1-methylpiperidin-4-yl)amino)benzo[b]thiophen-2-yl)prop-2-yn-1-yl)amino)-3- |
| | methoxy-N-methylbenzamide; |
| 211 | 4-((3-(3-(cyanomethyl)-7-((1-(dimethylglycyl)piperidin-4-yl)amino)benzo[b]thiophen-2-yl)prop-2-yn-1-yl) |
| | amino)-3-methoxy-N-methylbenzamide; |
| 212 | 4-((3-(7-((1-(dimethylglycyl)piperidin-4-yl)amino)-3-(thiazol-4-yl)benzo[b]thiophen-2-yl)prop-2-yn-1-yl) |
| | amino)-3-methoxy-N-methylbenzamide; |
| 213 | 4-((3-(7-((1-(dimethylglycyl)piperidin-4-yl)amino)-3-(2,2,2-trifluoroethyl)benzo[b]thiophen-2-yl)prop-2-yn- |
| | 1-yl)amino)-3-methoxy-N-methylbenzamide; |
| 214 | 4-((3-(3-(cyclopropylmethyl)-7-((1-(dimethylglycyl)piperidin-4-yl)amino)benzo[b]thiophen-2-yl)prop-2-yn- |
| | 1-yl)amino)-3-methoxy-N-methylbenzamide; |
| 215 | 4-((3-(3-(2,2-difluoroethyl)-7-((1-(dimethylglycyl)piperidin-4-yl)amino)benzo[b]thiophen-2-yl)prop-2-yn-1- |
| | yl)amino)-3-methoxy-N-methylbenzamide; |
| 216 | 4-((3-(7-((1-(dimethylglycyl)piperidin-4-yl)amino)-3-(1H-pyrrol-1-yl)benzo[b]thiophen-2-yl)prop-2-yn-1- |
| | yl)amino)-3-methoxy-N-methylbenzamide; |
| 217 | 4-((3-(7-((1-(dimethylglycyl)piperidin-4-yl)amino)-3-ethylbenzo[b]thiophen-2-yl)prop-2-yn-1-yl)amino)-3- |
| | methoxy-N-methylbenzamide; |
| 218 | 4-((3-(3-(cyanomethyl)-7-((3-fluoro-1-(2-hydroxy-3-methoxypropyl)piperidin-4-yl)amino)benzo[b]thiophen- |
| | 2-yl)prop-2-yn-1-yl)amino)-3-methoxy-N-methylbenzamide; |
| 219 | 4-((3-(7-((3-fluoro-1-(2-hydroxy-3-methoxypropyl)piperidin-4-yl)amino)-3-(thiazol-4-yl)benzo[b]thiophen- |
| | 2-yl)prop-2-yn-1-yl)amino)-3-methoxy-N-methylbenzamide; |
| 220 | 4-((3-(7-((3-fluoro-1-(2-hydroxy-3-methoxypropyl)piperidin-4-yl)amino)-3-(2,2,2-trifluoroethyl)benzo[b]thiophen- |
| | 2-yl)prop-2-yn-1-yl)amino)-3-methoxy-N-methylbenzamide; |
| 221 | 4-((3-(3-(cyclopropylmethyl)-7-((3-fluoro-1-(2-hydroxy-3-methoxypropyl)piperidin-4-yl)amino)benzo[b]thiophen- |
| | 2-yl)prop-2-yn-1-yl)amino)-3-methoxy-N-methylbenzamide; |
| 222 | 4-((3-(3-(2,2-difluoroethyl)-7-((3-fluoro-1-(2-hydroxy-3-methoxypropyl)piperidin-4-yl)amino)benzo[b]thiophen- |
| | 2-yl)prop-2-yn-1-yl)amino)-3-methoxy-N-methylbenzamide; |
| 223 | 4-((3-(7-((3-fluoro-1-(2-hydroxy-3-methoxypropyl)piperidin-4-yl)amino)-3-(1H-pyrrol-1-yl)benzo[b]thiophen- |
| | 2-yl)prop-2-yn-1-yl)amino)-3-methoxy-N-methylbenzamide; |
| 224 | 4-((3-(3-ethyl-7-((3-fluoro-1-(2-hydroxy-3-methoxypropyl)piperidin-4-yl)amino)benzo[b]thiophen-2-yl)prop- |
| | 2-yn-1-yl)amino)-3-methoxy-N-methylbenzamide; |
| 225 | 4-((3-(3-(cyanomethyl)-7-(((3R,4R)-3-fluoro-1-methylpiperidin-4-yl)amino)benzo[b]thiophen-2-yl)prop-2- |
| | yn-1-yl)amino)-3-methoxy-N-methylbenzamide; |
| 226 | 4-((3-(7-(((3R,4R)-3-fluoro-1-methylpiperidin-4-yl)amino)-3-(thiazol-4-yl)benzo[b]thiophen-2-yl)prop-2-yn- |
| | 1-yl)amino)-3-methoxy-N-methylbenzamide; |
| 227 | 4-((3-(7-(((3R,4R)-3-fluoro-1-methylpiperidin-4-yl)amino)-3-(2,2,2-trifluoroethyl)benzo[b]thiophen-2-yl)prop- |
| | 2-yn-1-yl)amino)-3-methoxy-N-methylbenzamide; |
| 228 | 4-((3-(3-(cyclopropylmethyl)-7-(((3R,4R)-3-fluoro-1-methylpiperidin-4-yl)amino)benzo[b]thiophen-2-yl)prop- |
| | 2-yn-1-yl)amino)-3-methoxy-N-methylbenzamide; |
| 229 | 4-((3-(3-(2,2-difluoroethyl)-7-(((3R,4R)-3-fluoro-1-methylpiperidin-4-yl)amino)benzo[b]thiophen-2-yl)prop- |
| | 2-yn-1-yl)amino)-3-methoxy-N-methylbenzamide; |
| 230 | 4-((3-(7-(((3R,4R)-3-fluoro-1-methylpiperidin-4-yl)amino)-3-(1H-pyrrol-1-yl)benzo[b]thiophen-2-yl)prop- |
| | 2-yn-1-yl)amino)-3-methoxy-N-methylbenzamide; |
| 231 | 4-((3-(3-ethyl-7-(((3R,4R)-3-fluoro-1-methylpiperidin-4-yl)amino)benzo[b]thiophen-2-yl)prop-2-yn-1-yl) |
| | amino)-3-methoxy-N-methylbenzamide; |
| 232 | 4-((3-(3-(cyanomethyl)-7-(((3S,4S)-3-fluoro-1-methylpiperidin-4-yl)amino)benzo[b]thiophen-2-yl)prop-2-yn- |
| | 1-yl)amino)-3-methoxy-N-methylbenzamide; |
| 233 | 4-((3-(7-(((3S,4S)-3-fluoro-1-methylpiperidin-4-yl)amino)-3-(thiazol-4-yl)benzo[b]thiophen-2-yl)prop-2-yn- |
| | 1-yl)amino)-3-methoxy-N-methylbenzamide; |
| 234 | 4-((3-(7-(((3S,4S)-3-fluoro-1-methylpiperidin-4-yl)amino)-3-(2,2,2-trifluoroethyl)benzo[b]thiophen-2-yl)prop- |
| | 2-yn-1-yl)amino)-3-methoxy-N-methylbenzamide; |
| 235 | 4-((3-(3-(cyclopropylmethyl)-7-(((3S,4S)-3-fluoro-1-methylpiperidin-4-yl)amino)benzo[b]thiophen-2-yl)prop- |
| | 2-yn-1-yl)amino)-3-methoxy-N-methylbenzamide; |
| 236 | 4-((3-(3-(2,2-difluoroethyl)-7-(((3S,4S)-3-fluoro-1-methylpiperidin-4-yl)amino)benzo[b]thiophen-2-yl)prop- |
| | 2-yn-1-yl)amino)-3-methoxy-N-methylbenzamide; |
| 237 | 4-((3-(7-(((3S,4S)-3-fluoro-1-methylpiperidin-4-yl)amino)-3-(1H-pyrrol-1-yl)benzo[b]thiophen-2-yl)prop-2- |
| | yn-1-yl)amino)-3-methoxy-N-methylbenzamide; |
| 238 | 4-((3-(3-ethyl-7-(((3S,4S)-3-fluoro-1-methylpiperidin-4-yl)amino)benzo[b]thiophen-2-yl)prop-2-yn-1-yl)amino)- |
| | 3-methoxy-N-methylbenzamide; |
| 239 | 4-((3-(3-(cyanomethyl)-7-((4-(dimethylamino)cyclohexyl)amino)benzo[b]thiophen-2-yl)prop-2-yn-1-yl)amino)- |
| | 3-methoxy-N-methylbenzamide; |
| 240 | 4-((3-(7-((4-(dimethylamino)cyclohexyl)amino)-3-(thiazol-4-yl)benzo[b]thiophen-2-yl)prop-2-yn-1-yl)amino)- |
| | 3-methoxy-N-methylbenzamide; |
| 241 | 4-((3-(7-((4-(dimethylamino)cyclohexyl)amino)-3-(2,2,2-trifluoroethyl)benzo[b]thiophen-2-yl)prop-2-yn-1- |
| | yl)amino)-3-methoxy-N-methylbenzamide; |
| 242 | 4-((3-(3-(cyclopropylmethyl)-7-((4-(dimethylamino)cyclohexyl)amino)benzo[b]thiophen-2-yl)prop-2-yn-1- |
| | yl)amino)-3-methoxy-N-methylbenzamide; |
| 243 | 4-((3-(3-(2,2-difluoroethyl)-7-((4-(dimethylamino)cyclohexyl)amino)benzo[b]thiophen-2-yl)prop-2-yn-1-yl) |
| | amino)-3-methoxy-N-methylbenzamide; |
| 244 | 4-((3-(7-((4-(dimethylamino)cyclohexyl)amino)-3-(1H-pyrrol-1-yl)benzo[b]thiophen-2-yl)prop-2-yn-1-yl)amino)- |
| | 3-methoxy-N-methylbenzamide; |
| 245 | 4-((3-(7-((4-(dimethylamino)cyclohexyl)amino)-3-ethylbenzo[b]thiophen-2-yl)prop-2-yn-1-yl)amino)-3-methoxy- |
| | N-methylbenzamide; |
| 246 | 4-((3-(3-(cyanomethyl)-7-((2-(dimethylamino)ethyl)amino)benzo[b]thiophen-2-yl)prop-2-yn-1-yl)amino)-3- |
| | methoxy-N-methylbenzamide; |
| 247 | 4-((3-(7-((2-(dimethylamino)ethyl)amino)-3-(thiazol-4-yl)benzo[b]thiophen-2-yl)prop-2-yn-1-yl)amino)-3- |
| | methoxy-N-methylbenzamide; |
| 248 | 4-((3-(7-((2-(dimethylamino)ethyl)amino)-3-(2,2,2-trifluoroethyl)benzo[b]thiophen-2-yl)prop-2-yn-1-yl)amino)- |
| | 3-methoxy-N-methylbenzamide; |
| 249 | 4-((3-(3-(cyclopropylmethyl)-7-((2-(dimethylamino)ethyl)amino)benzo[b]thiophen-2-yl)prop-2-yn-1-yl)amino)- |
| | 3-methoxy-N-methylbenzamide; |
| 250 | 4-((3-(3-(2,2-difluoroethyl)-7-((2-(dimethylamino)ethyl)amino)benzo[b]thiophen-2-yl)prop-2-yn-1-yl)amino)- |
| | 3-methoxy-N-methylbenzamide; |
| 251 | 4-((3-(7-((2-(dimethylamino)ethyl)amino)-3-(1H-pyrrol-1-yl)benzo[b]thiophen-2-yl)prop-2-yn-1-yl)amino)- |
| | 3-methoxy-N-methylbenzamide; |
| 252 | 4-((3-(7-((2-(dimethylamino)ethyl)amino)-3-ethylbenzo[b]thiophen-2-yl)prop-2-yn-1-yl)amino)-3-methoxy- |
| | N-methylbenzamide; |
| 253 | 4-((3-(3-(cyanomethyl)-7-(((3S,4R)-3-fluoro-1-methylpiperidin-4-yl)amino)benzo[b]thiophen-2-yl)prop-2- |
| | yn-1-yl)amino)-3-methoxy-N-methylbenzamide; |
| 254 | 4-((3-(7-(((3S,4R)-3-fluoro-1-methylpiperidin-4-yl)amino)-3-(thiazol-4-yl)benzo[b]thiophen-2-yl)prop-2- |
| | yn-1-yl)amino)-3-methoxy-N-methylbenzamide; |
| 255 | 4-((3-(7-(((3S,4R)-3-fluoro-1-methylpiperidin-4-yl)amino)-3-(2,2,2-trifluoroethyl)benzo[b]thiophen-2-yl)prop- |
| | 2-yn-1-yl)amino)-3-methoxy-N-methylbenzamide; |
| 256 | 4-((3-(3-(cyclopropylmethyl)-7-(((3S,4R)-3-fluoro-1-methylpiperidin-4-yl)amino)benzo[b]thiophen-2-yl)prop- |
| | 2-yn-1-yl)amino)-3-methoxy-N-methylbenzamide; |
| 257 | 4-((3-(3-(2,2-difluoroethyl)-7-(((3S,4R)-3-fluoro-1-methylpiperidin-4-yl)amino)benzo[b]thiophen-2-yl)prop- |
| | 2-yn-1-yl)amino)-3-methoxy-N-methylbenzamide; |
| 258 | 4-((3-(7-(((3S,4R)-3-fluoro-1-methylpiperidin-4-yl)amino)-3-(1H-pyrrol-1-yl)benzo[b]thiophen-2-yl)prop-2- |
| | yn-1-yl)amino)-3-methoxy-N-methylbenzamide; |
| 259 | 4-((3-(3-ethyl-7-(((3S,4R)-3-fluoro-1-methylpiperidin-4-yl)amino)benzo[b]thiophen-2-yl)prop-2-yn-1-yl)amino)- |
| | 3-methoxy-N-methylbenzamide; |
| 260 | 4-((3-(3-(cyanomethyl)-7-((1-methylpiperidin-4-yl)amino)benzo[b]thiophen-2-yl)prop-2-yn-1-yl)amino)-3- |
| | methoxy-N-methylbenzamide; |
| 261 | 3-methoxy-N-methyl-4-((3-(7-((1-methylpiperidin-4-yl)amino)-3-(thiazol-4-yl)benzo[b]thiophen-2-yl)prop- |
| | 2-yn-1-yl)amino)benzamide; |
| 262 | 3-methoxy-N-methyl-4-((3-(7-((1-methylpiperidin-4-yl)amino)-3-(2,2,2-trifluoroethyl)benzo[b]thiophen-2- |
| | yl)prop-2-yn-1-yl)amino)benzamide; |
| 263 | 4-((3-(3-(cyclopropylmethyl)-7-((1-methylpiperidin-4-yl)amino)benzo[b]thiophen-2-yl)prop-2-yn-1-yl)amino)- |
| | 3-methoxy-N-methylbenzamide; |
| 264 | 4-((3-(3-(2,2-difluoroethyl)-7-((1-methylpiperidin-4-yl)amino)benzo[b]thiophen-2-yl)prop-2-yn-1-yl)amino)- |
| | 3-methoxy-N-methylbenzamide; |
| 265 | 3-methoxy-N-methyl-4-((3-(7-((1-methylpiperidin-4-yl)amino)-3-(1H-pyrrol-1-yl)benzo[b]thiophen-2-yl)prop- |
| | 2-yn-1-yl)amino)benzamide; |
| 266 | 4-((3-(3-ethyl-7-((1-methylpiperidin-4-yl)amino)benzo[b]thiophen-2-yl)prop-2-yn-1-yl)amino)-3-methoxy- |
| | N-methylbenzamide; |
| 267 | 4-((3-(3-(cyanomethyl)-7-((tetrahydro-2H-pyran-4-yl)amino)benzo[b]thiophen-2-yl)prop-2-yn-1-yl)amino)- |
| | 3-methoxy-N-methylbenzamide; |
| 268 | 3-methoxy-N-methyl-4-((3-(7-((tetrahydro-2H-pyran-4-yl)amino)-3-(thiazol-4-yl)benzo[b]thiophen-2-yl)prop- |
| | 2-yn-1-yl)amino)benzamide; |
| 269 | 3-methoxy-N-methyl-4-((3-(7-((tetrahydro-2H-pyran-4-yl)amino)-3-(2,2,2-trifluoroethyl)benzo[b]thiophen- |
| | 2-yl)prop-2-yn-1-yl)amino)benzamide; |
| 270 | 4-((3-(3-(cyclopropylmethyl)-7-((tetrahydro-2H-pyran-4-yl)amino)benzo[b]thiophen-2-yl)prop-2-yn-1-yl)amino)- |
| | 3-methoxy-N-methylbenzamide; |
| 271 | 4-((3-(3-(2,2-difluoroethyl)-7-((tetrahydro-2H-pyran-4-yl)amino)benzo[b]thiophen-2-yl)prop-2-yn-1-yl)amino)- |
| | 3-methoxy-N-methylbenzamide; |
| 272 | 4-((3-(3-(1H-pyrrol-1-yl)-7-((tetrahydro-2H-pyran-4-yl)amino)benzo[b]thiophen-2-yl)prop-2-yn-1-yl)amino)- |
| | 3-methoxy-N-methylbenzamide; |
| 273 | 4-((3-(3-ethyl-7-((tetrahydro-2H-pyran-4-yl)amino)benzo[b]thiophen-2-yl)prop-2-yn-1-yl)amino)-3-methoxy- |
| | N-methylbenzamide; |
| 274 | 2-(7-((3,3-difluoro-1-methylpiperidin-4-yl)amino)-2-(3-((2-methoxy-4-(methylsulfonyl)phenyl)amino)prop- |
| | 1-yn-1-yl)benzo[b]thiophen-3-yl)acetonitrile; |
| 275 | 3,3-difluoro-N-(2-(3-((2-methoxy-4-(methylsulfonyl)phenyl)amino)prop-1-yn-1-yl)-3-(thiazol-4-yl)benzo[b] |
| | thiophen-7-yl)-1-methylpiperidin-4-amine; |
| 276 | 3,3-difluoro-N-(2-(3-((2-methoxy-4-(methylsulfonyl)phenyl)amino)prop-1-yn-1-yl)-3-(2,2,2-trifluoroethyl) |
| | benzo[b]thiophen-7-yl)-1-methylpiperidin-4-amine; |
| 277 | N-(3-(cyclopropylmethyl)-2-(3-((2-methoxy-4-(methylsulfonyl)phenyl)amino)prop-1-yn-1-yl)benzo[b]thiophen- |
| | 7-yl)-3,3-difluoro-1-methylpiperidin-4-amine; |
| 278 | N-(3-(2,2-difluoroethyl)-2-(3-((2-methoxy-4-(methylsulfonyl)phenyl)amino)prop-1-yn-1-yl)benzo[b]thiophen- |
| | 7-yl)-3,3-difluoro-1-methylpiperidin-4-amine; |
| 279 | 3,3-difluoro-N-(2-(3-((2-methoxy-4-(methylsulfonyl)phenyl)amino)prop-1-yn-1-yl)-3-(1H-pyrrol-1-yl)benzo |
| | [b]thiophen-7-yl)-1-methylpiperidin-4-amine; |
| 280 | N-(3-ethyl-2-(3-((2-methoxy-4-(methylsulfonyl)phenyl)amino)prop-1-yn-1-yl)benzo[b]thiophen-7-yl)-3,3-difluoro- |
| | 1-methylpiperidin-4-amine; |
| 281 | 2-(7-((1,1-dioxidotetrahydro-2H-thiopyran-4-yl)amino)-2-(3-((2-methoxy-4-(methylsulfonyl)phenyl)amino) |
| | prop-1-yn-1-yl)benzo[b]thiophen-3-yl)acetonitrile; |
| 282 | 4-((2-(3-((2-methoxy-4-(methylsulfonyl)phenyl)amino)prop-1-yn-1-yl)-3-(thiazol-4-yl)benzo[b]thiophen-7- |
| | yl)amino)tetrahydro-2H-thiopyran 1,1-dioxide; |
| 283 | 4-((2-(3-((2-methoxy-4-(methylsulfonyl)phenyl)amino)prop-1-yn-1-yl)-3-(2,2,2-trifluoroethyl)benzo[b]thiophen- |
| | 7-yl)amino)tetrahydro-2H-thiopyran 1,1-dioxide; |
| 284 | 4-((3-(cyclopropylmethyl)-2-(3-((2-methoxy-4-(methylsulfonyl)phenyl)amino)prop-1-yn-1-yl)benzo[b]thiophen- |
| | 7-yl)amino)tetrahydro-2H-thiopyran 1,1-dioxide; |
| 285 | 4-((3-(2,2-difluoroethyl)-2-(3-((2-methoxy-4-(methylsulfonyl)phenyl)amino)prop-1-yn-1-yl)benzo[b]thiophen- |
| | 7-yl)amino)tetrahydro-2H-thiopyran 1,1-dioxide; |
| 286 | 4-((2-(3-((2-methoxy-4-(methylsulfonyl)phenyl)amino)prop-1-yn-1-yl)-3-(1H-pyrrol-1-yl)benzo[b]thiophen- |
| | 7-yl)amino)tetrahydro-2H-thiopyran 1,1-dioxide; |
| 287 | 4-((3-ethyl-2-(3-((2-methoxy-4-(methylsulfonyl)phenyl)amino)prop-1-yn-1-yl)benzo[b]thiophen-7-yl)amino) |
| | tetrahydro-2H-thiopyran 1,1-dioxide; |
| 288 | 2-(7-(((3R,4S)-3-fluoro-1-methylpiperidin-4-yl)amino)-2-(3-((2-methoxy-4-(methylsulfonyl)phenyl)amino) |
| | prop-1-yn-1-yl)benzo[b]thiophen-3-yl)acetonitrile; |
| 289 | (3R,4S)-3-fluoro-N-(2-(3-((2-methoxy-4-(methylsulfonyl)phenyl)amino)prop-1-yn-1-yl)-3-(thiazol-4-yl)benzo |
| | [b]thiophen-7-yl)-1-methylpiperidin-4-amine; |
| 290 | (3R,4S)-3-fluoro-N-(2-(3-((2-methoxy-4-(methylsulfonyl)phenyl)amino)prop-1-yn-1-yl)-3-(2,2,2-trifluoroethyl) |
| | benzo[b]thiophen-7-yl)-1-methylpiperidin-4-amine; |
| 291 | (3R,4S)-N-(3-(cyclopropylmethyl)-2-(3-((2-methoxy-4-(methylsulfonyl)phenyl)amino)prop-1-yn-1-yl)benzo |
| | [b]thiophen-7-yl)-3-fluoro-1-methylpiperidin-4-amine; |
| 292 | (3R,4S)-N-(3-(2,2-difluoroethyl)-2-(3-((2-methoxy-4-(methylsulfonyl)phenyl)amino)prop-1-yn-1-yl)benzo[b] |
| | thiophen-7-yl)-3-fluoro-1-methylpiperidin-4-amine; |
| 293 | (3R,4S)-3-fluoro-N-(2-(3-((2-methoxy-4-(methylsulfonyl)phenyl)amino)prop-1-yn-1-yl)-3-(1H-pyrrol-1-yl) |
| | benzo[b]thiophen-7-yl)-1-methylpiperidin-4-amine; |
| 294 | (3R,4S)-N-(3-ethyl-2-(3-((2-methoxy-4-(methylsulfonyl)phenyl)amino)prop-1-yn-1-yl)benzo[b]thiophen-7- |
| | yl)-3-fluoro-1-methylpiperidin-4-amine; |
| 295 | 2-(7-((3-fluoro-1-methylpiperidin-4-yl)amino)-2-(3-((2-methoxy-4-(methylsulfonyl)phenyl)amino)prop-1-yn- |
| | 1-yl)benzo[b]thiophen-3-yl)acetonitrile; |
| 296 | 3-fluoro-N-(2-(3-((2-methoxy-4-(methylsulfonyl)phenyl)amino)prop-1-yn-1-yl)-3-(thiazol-4-yl)benzo[b]thiophen- |
| | 7-yl)-1-methylpiperidin-4-amine; |
| 297 | 3-fluoro-N-(2-(3-((2-methoxy-4-(methylsulfonyl)phenyl)amino)prop-1-yn-1-yl)-3-(2,2,2-trifluoroethyl)benzo |
| | [b]thiophen-7-yl)-1-methylpiperidin-4-amine; |
| 298 | N-(3-(cyclopropylmethyl)-2-(3-((2-methoxy-4-(methylsulfonyl)phenyl)amino)prop-1-yn-1-yl)benzo[b]thiophen- |
| | 7-yl)-3-fluoro-1-methylpiperidin-4-amine; |
| 299 | N-(3-(2,2-difluoroethyl)-2-(3-((2-methoxy-4-(methylsulfonyl)phenyl)amino)prop-1-yn-1-yl)benzo[b]thiophen- |
| | 7-yl)-3-fluoro-1-methylpiperidin-4-amine; |
| 300 | 3-fluoro-N-(2-(3-((2-methoxy-4-(methylsulfonyl)phenyl)amino)prop-1-yn-1-yl)-3-(1H-pyrrol-1-yl)benzo[b] |
| | thiophen-7-yl)-1-methylpiperidin-4-amine; |
| 301 | N-(3-ethyl-2-(3-((2-methoxy-4-(methylsulfonyl)phenyl)amino)prop-1-yn-1-yl)benzo[b]thiophen-7-yl)-3-fluoro- |
| | 1-methylpiperidin-4-amine; |
| 302 | 2-(7-((1-(dimethylglycyl)piperidin-4-yl)amino)-2-(3-((2-methoxy-4-(methylsulfonyl)phenyl)amino)prop-1- |
| | yn-1-yl)benzo[b]thiophen-3-yl)acetonitrile; |
| 303 | 2-(dimethylamino)-1-(4-((2-(3-((2-methoxy-4-(methylsulfonyl)phenyl)amino)prop-1-yn-1-yl)-3-(thiazol-4- |
| | yl)benzo[b]thiophen-7-yl)amino)piperidin-1-yl)ethan-1-one; |
| 304 | 2-(dimethylamino)-1-(4-((2-(3-((2-methoxy-4-(methylsulfonyl)phenyl)amino)prop-1-yn-1-yl)-3-(2,2,2-trifluoroethyl) |
| | benzo[b]thiophen-7-yl)amino)piperidin-1-yl)ethan-1-one; |
| 305 | 1-(4-((3-(cyclopropylmethyl)-2-(3-((2-methoxy-4-(methylsulfonyl)phenyl)amino)prop-1-yn-1-yl)benzo[b]thiophen- |
| | 7-yl)amino)piperidin-1-yl)-2-(dimethylamino)ethan-1-one; |
| 306 | 1-(4-((3-(2,2-difluoroethyl)-2-(3-((2-methoxy-4-(methylsulfonyl)phenyl)amino)prop-1-yn-1-yl)benzo[b]thiophen- |
| | 7-yl)amino)piperidin-1-yl)-2-(dimethylamino)ethan-1-one; |
| 307 | 2-(dimethylamino)-1-(4-((2-(3-((2-methoxy-4-(methylsulfonyl)phenyl)amino)prop-1-yn-1-yl)-3-(1H-pyrrol- |
| | 1-yl)benzo[b]thiophen-7-yl)amino)piperidin-1-yl)ethan-1-one; |
| 308 | 2-(dimethylamino)-1-(4-((3-ethyl-2-(3-((2-methoxy-4-(methylsulfonyl)phenyl)amino)prop-1-yn-1-yl)benzo |
| | [b]thiophen-7-yl)amino)piperidin-1-yl)ethan-1-one; |
| 309 | 2-(7-((3-fluoro-1-(2-hydroxy-3-methoxypropyl)piperidin-4-yl)amino)-2-(3-((2-methoxy-4-(methylsulfonyl) |
| | phenyl)amino)prop-1-yn-1-yl)benzo[b]thiophen-3-yl)acetonitrile; |
| 310 | 1-(3-fluoro-4-((2-(3-((2-methoxy-4-(methylsulfonyl)phenyl)amino)prop-1-yn-1-yl)-3-(thiazol-4-yl)benzo[b] |
| | thiophen-7-yl)amino)piperidin-1-yl)-3-methoxypropan-2-o1; |
| 311 | 1-(3-fluoro-4-((2-(3-((2-methoxy-4-(methylsulfonyl)phenyl)amino)prop-1-yn-1-yl)-3-(2,2,2-trifluoroethyl) |
| | benzo[b]thiophen-7-yl)amino)piperidin-1-yl)-3-methoxypropan-2-ol; |
| 312 | 1-(4-((3-(cyclopropylmethyl)-2-(3-((2-methoxy-4-(methylsulfonyl)phenyl)amino)prop-1-yn-1-yl)benzo[b] |
| | thiophen-7-yl)amino)-3-fluoropiperidin-1-yl)-3-methoxypropan-2-ol; |
| 313 | 1-(4-((3-(2,2-difluoroethyl)-2-(3-((2-methoxy-4-(methylsulfonyl)phenyl)amino)prop-1-yn-1-yl)benzo[b]thiophen- |
| | 7-yl)amino)-3-fluoropiperidin-1-yl)-3-methoxypropan-2-ol; |
| 314 | 1-(3-fluoro-4-((2-(3-((2-methoxy-4-(methylsulfonyl)phenyl)amino)prop-1-yn-1-yl)-3-(1H-pyrrol-1-yl)benzo |
| | [b]thiophen-7-yl)amino)piperidin-1-yl)-3-methoxypropan-2-ol; |
| 315 | 1-(4-((3-ethyl-2-(3-((2-methoxy-4-(methylsulfonyl)phenyl)amino)prop-1-yn-1-yl)benzo[b]thiophen-7-yl)amino)- |
| | 3-fluoropiperidin-1-yl)-3-methoxypropan-2-ol; |
| 316 | 2-(7-(((3R,4R)-3-fluoro-1-methylpiperidin-4-yl)amino)-2-(3-((2-methoxy-4-(methylsulfonyl)phenyl)amino) |
| | prop-1-yn-1-yl)benzo[b]thiophen-3-yl)acetonitrile; |
| 317 | (3R,4R)-3-fluoro-N-(2-(3-((2-methoxy-4-(methylsulfonyl)phenyl)amino)prop-1-yn-1-yl)-3-(thiazol-4-yl)benzo |
| | [b]thiophen-7-yl)-1-methylpiperidin-4-amine; |
| 318 | (3R,4R)-3-fluoro-N-(2-(3-((2-methoxy-4-(methylsulfonyl)phenyl)amino)prop-1-yn-1-yl)-3-(2,2,2-trifluoroethyl) |
| | benzo[b]thiophen-7-yl)-1-methylpiperidin-4-amine; |
| 319 | (3R,4R)-N-(3-(cyclopropylmethyl)-2-(3-((2-methoxy-4-(methylsulfonyl)phenyl)amino)prop-1-yn-1-yl)benzo |
| | [b]thiophen-7-yl)-3-fluoro-1-methylpiperidin-4-amine; |
| 320 | (3R,4R)-N-(3-(2,2-difluoroethyl)-2-(3-((2-methoxy-4-(methylsulfonyl)phenyl)amino)prop-1-yn-1-yl)benzo[b] |
| | thiophen-7-yl)-3-fluoro-1-methylpiperidin-4-amine; |
| 321 | (3R,4R)-3-fluoro-N-(2-(3-((2-methoxy-4-(methylsulfonyl)phenyl)amino)prop-1-yn-1-yl)-3-(1H-pyrrol-1-yl) |
| | benzo[b]thiophen-7-yl)-1-methylpiperidin-4-amine; |
| 322 | (3R,4R)-N-(3-ethyl-2-(3-((2-methoxy-4-(methylsulfonyl)phenyl)amino)prop-1-yn-1-yl)benzo[b]thiophen-7- |
| | yl)-3-fluoro-1-methylpiperidin-4-amine; |
| 323 | 2-(7-(((3S,4S)-3-fluoro-1-methylpiperidin-4-yl)amino)-2-(3-((2-methoxy-4-(methylsulfonyl)phenyl)amino) |
| | prop-1-yn-1-yl)benzo[b]thiophen-3-yl)acetonitrile; |
| 324 | (3S,4S)-3-fluoro-N-(2-(3-((2-methoxy-4-(methylsulfonyl)phenyl)amino)prop-1-yn-1-yl)-3-(thiazol-4-yl)benzo |
| | [b]thiophen-7-yl)-1-methylpiperidin-4-amine; |
| 325 | (3S,4S)-3-fluoro-N-(2-(3-((2-methoxy-4-(methylsulfonyl)phenyl)amino)prop-1-yn-1-yl)-3-(2,2,2-trifluoroethyl) |
| | benzo[b]thiophen-7-yl)-1-methylpiperidin-4-amine; |
| 326 | (3S,4S)-N-(3-(cyclopropylmethyl)-2-(3-((2-methoxy-4-(methylsulfonyl)phenyl)amino)prop-1-yn-1-yl)benzo |
| | [b]thiophen-7-yl)-3-fluoro-1-methylpiperidin-4-amine; |
| 327 | (3S,4S)-N-(3-(2,2-difluoroethyl)-2-(3-((2-methoxy-4-(methylsulfonyl)phenyl)amino)prop-1-yn-1-yl)benzo |
| | [b]thiophen-7-yl)-3-fluoro-1-methylpiperidin-4-amine; |
| 328 | (3S,4S)-3-fluoro-N-(2-(3-((2-methoxy-4-(methylsulfonyl)phenyl)amino)prop-1-yn-1-yl)-3-(1H-pyrrol-1-yl) |
| | benzo[b]thiophen-7-yl)-1-methylpiperidin-4-amine; |
| 329 | (3S,4S)-N-(3-ethyl-2-(3-((2-methoxy-4-(methylsulfonyl)phenyl)amino)prop-1-yn-1-yl)benzo[b]thiophen-7- |
| | yl)-3-fluoro-1-methylpiperidin-4-amine; |
| 330 | 2-(7-((4-(dimethylamino)cyclohexyl)amino)-2-(3-((2-methoxy-4-(methylsulfonyl)phenyl)amino)prop-1-yn- |
| | 1-yl)benzo[b]thiophen-3-yl)acetonitrile; |
| 331 | N1-(2-(3-((2-methoxy-4-(methylsulfonyl)phenyl)amino)prop-1-yn-1-yl)-3-(thiazol-4-yl)benzo[b]thiophen-7- |
| | yl)-N4,N4-dimethylcyclohexane-1,4-diamine; |
| 332 | N1-(2-(3-((2-methoxy-4-(methylsulfonyl)phenyl)amino)prop-1-yn-1-yl)-3-(2,2,2-trifluoroethyl)benzo[b]thiophen- |
| | 7-yl)-N4,N4-dimethylcyclohexane-1,4-diamine; |
| 333 | N1-(3-(cyclopropylmethyl)-2-(3-((2-methoxy-4-(methylsulfonyl)phenyl)amino)prop-1-yn-1-yl)benzo[b]thiophen- |
| | 7-yl)-N4,N4-dimethylcyclohexane-1,4-diamine; |
| 334 | N1-(3-(2,2-difluoroethyl)-2-(3-((2-methoxy-4-(methylsulfonyl)phenyl)amino)prop-1-yn-1-yl)benzo[b]thiophen- |
| | 7-yl)-N4,N4-dimethylcyclohexane-1,4-diamine; |
| 335 | N1-(2-(3-((2-methoxy-4-(methylsulfonyl)phenyl)amino)prop-1-yn-1-yl)-3-(1H-pyrrol-1-yl)benzo[b]thiophen- |
| | 7-yl)-N4,N4-dimethylcyclohexane-1,4-diamine; |
| 336 | N1-(3-ethyl-2-(3-((2-methoxy-4-(methylsulfonyl)phenyl)amino)prop-1-yn-1-yl)benzo[b]thiophen-7-yl)- |
| | N4,N4-dimethylcyclohexane-1,4-diamine; |
| 337 | 2-(7-((2-(dimethylamino)ethyl)amino)-2-(3-((2-methoxy-4-(methylsulfonyl)phenyl)amino)prop-1-yn-1-yl)benzo |
| | [b]thiophen-3-yl)acetonitrile; |
| 338 | N1-(2-(3-((2-methoxy-4-(methylsulfonyl)phenyl)amino)prop-1-yn-1-yl)-3-(thiazol-4-yl)benzo[b]thiophen-7- |
| | yl)-N2,N2-dimethylethane-1,2-diamine; |
| 339 | N1-(2-(3-((2-methoxy-4-(methylsulfonyl)phenyl)amino)prop-1-yn-1-yl)-3-(2,2,2-trifluoroethyl)benzo[b]thiophen- |
| | 7-yl)-N2,N2-dimethylethane-1,2-diamine; |
| 340 | N1-(3-(cyclopropylmethyl)-2-(3-((2-methoxy-4-(methylsulfonyl)phenyl)amino)prop-1-yn-1-yl)benzo[b]thiophen- |
| | 7-yl)-N2,N2-dimethylethane-1,2-diamine; |
| 341 | N1-(3-(2,2-difluoroethyl)-2-(3-((2-methoxy-4-(methylsulfonyl)phenyl)amino)prop-1-yn-1-yl)benzo[b]thiophen- |
| | 7-yl)-N2,N2-dimethylethane-1,2-diamine; |
| 342 | N1-(2-(3-((2-methoxy-4-(methylsulfonyl)phenyl)amino)prop-1-yn-1-yl)-3-(1H-pyrrol-1-yl)benzo[b]thiophen- |
| | 7-yl)-N2,N2-dimethylethane-1,2-diamine; |
| 343 | N1-(3-ethyl-2-(3-((2-methoxy-4-(methylsulfonyl)phenyl)amino)prop-1-yn-1-yl)benzo[b]thiophen-7-yl)- |
| | N2,N2-dimethylethane-1,2-diamine; |
| 344 | 2-(7-(((3S,4R)-3-fluoro-1-methylpiperidin-4-yl)amino)-2-(3-((2-methoxy-4-(methylsulfonyl)phenyl)amino) |
| | prop-1-yn-1-yl)benzo[b]thiophen-3-yl)acetonitrile; |
| 345 | (3S,4R)-3-fluoro-N-(2-(3-((2-methoxy-4-(methylsulfonyl)phenyl)amino)prop-1-yn-1-yl)-3-(thiazol-4-yl)benzo |
| | [b]thiophen-7-yl)-1-methylpiperidin-4-amine; |
| 346 | (3S,4R)-3-fluoro-N-(2-(3-((2-methoxy-4-(methylsulfonyl)phenyl)amino)prop-1-yn-1-yl)-3-(2,2,2-trifluoroethyl) |
| | benzo[b]thiophen-7-yl)-1-methylpiperidin-4-amine; |
| 347 | (3S,4R)-N-(3-(cyclopropylmethyl)-2-(3-((2-methoxy-4-(methylsulfonyl)phenyl)amino)prop-1-yn-1-yl)benzo |
| | [b]thiophen-7-yl)-3-fluoro-1-methylpiperidin-4-amine; |
| 348 | (3S,4R)-N-(3-(2,2-difluoroethyl)-2-(3-((2-methoxy-4-(methylsulfonyl)phenyl)amino)prop-1-yn-1-yl)benzo[b] |
| | thiophen-7-yl)-3-fluoro-1-methylpiperidin-4-amine; |
| 349 | (3S,4R)-3-fluoro-N-(2-(3-((2-methoxy-4-(methylsulfonyl)phenyl)amino)prop-1-yn-1-yl)-3-(1H-pyrrol-1-yl) |
| | benzo[b]thiophen-7-yl)-1-methylpiperidin-4-amine; |
| 350 | (3S,4R)-N-(3-ethyl-2-(3-((2-methoxy-4-(methylsulfonyl)phenyl)amino)prop-1-yn-1-yl)benzo[b]thiophen-7- |
| | yl)-3-fluoro-1-methylpiperidin-4-amine; |
| 351 | 2-(2-(3-((2-methoxy-4-(methylsulfonyl)phenyl)amino)prop-1-yn-1-yl)-7-((1-methylpiperidin-4-yl)amino)benzo |
| | [b]thiophen-3-yl)acetonitrile; |
| 352 | N-(2-(3-((2-methoxy-4-(methylsulfonyl)phenyl)amino)prop-1-yn-1-yl)-3-(thiazol-4-yl)benzo[b]thiophen-7- |
| | yl)-1-methylpiperidin-4-amine; |
| 353 | N-(2-(3-((2-methoxy-4-(methylsulfonyl)phenyl)amino)prop-1-yn-1-yl)-3-(2,2,2-trifluoroethyl)benzo[b]thiophen- |
| | 7-yl)-1-methylpiperidin-4-amine; |
| 354 | N-(3-(cyclopropylmethyl)-2-(3-((2-methoxy-4-(methylsulfonyl)phenyl)amino)prop-1-yn-1-yl)benzo[b]thiophen- |
| | 7-yl)-1-methylpiperidin-4-amine; |
| 355 | N-(3-(2,2-difluoroethyl)-2-(3-((2-methoxy-4-(methylsulfonyl)phenyl)amino)prop-1-yn-1-yl)benzo[b]thiophen- |
| | 7-yl)-1-methylpiperidin-4-amine; |
| 356 | N-(2-(3-((2-methoxy-4-(methylsulfonyl)phenyl)amino)prop-1-yn-1-yl)-3-(1H-pyrrol-1-yl)benzo[b]thiophen- |
| | 7-yl)-1-methylpiperidin-4-amine; |
| 357 | N-(3-ethyl-2-(3-((2-methoxy-4-(methylsulfonyl)phenyl)amino)prop-1-yn-1-yl)benzo[b]thiophen-7-yl)-1- |
| | methylpiperidin-4-amine; |
| 358 | 2-(2-(3-((2-methoxy-4-(methylsulfonyl)phenyl)amino)prop-1-yn-1-yl)-7-((tetrahydro-2H-pyran-4-yl)amino) |
| | benzo[b]thiophen-3-yl)acetonitrile; |
| 359 | N-(2-(3-((2-methoxy-4-(methylsulfonyl)phenyl)amino)prop-1-yn-1-yl)-3-(thiazol-4-yl)benzo[b]thiophen-7- |
| | yl)tetrahydro-2H-pyran-4-amine; |
| 360 | N-(2-(3-((2-methoxy-4-(methylsulfonyl)phenyl)amino)prop-1-yn-1-yl)-3-(2,2,2-trifluoroethyl)benzo[b]thiophen- |
| | 7-yl)tetrahydro-2H-pyran-4-amine; |
| 361 | N-(3-(cyclopropylmethyl)-2-(3-((2-methoxy-4-(methylsulfonyl)phenyl)amino)prop-1-yn-1-yl)benzo[b]thiophen- |
| | 7-yl)tetrahydro-2H-pyran-4-amine; |
| 362 | N-(3-(2,2-difluoroethyl)-2-(3-((2-methoxy-4-(methylsulfonyl)phenyl)amino)prop-1-yn-1-yl)benzo[b]thiophen- |
| | 7-yl)tetrahydro-2H-pyran-4-amine; |
| 363 | N-(2-(3-((2-methoxy-4-(methylsulfonyl)phenyl)amino)prop-1-yn-1-yl)-3-(1H-pyrrol-1-yl)benzo[b]thiophen- |
| | 7-yl)tetrahydro-2H-pyran-4-amine; |
| 364 | N-(3-ethyl-2-(3-((2-methoxy-4-(methylsulfonyl)phenyl)amino)prop-1-yn-1-yl)benzo[b]thiophen-7-yl)tetrahydro- |
| | 2H-pyran-4-amine; |
| 365 | 4-((3-(3-(cyanomethyl)-7-((3,3-difluoro-1-methylpiperidin-4-yl)amino)benzofuran-2-yl)prop-2-yn-1-yl)amino)- |
| | 3-methoxybenzenesulfonamide; |
| 366 | 2-(7-((3,3-difluoro-1-methylpiperidin-4-yl)amino)-2-(3-((5-fluoro-2-methoxy-4-(methylsulfonyl)phenyl)amino) |
| | prop-1-yn-1-yl)benzofuran-3-yl)acetonitrile; |
| 367 | 4-((3-(3-(cyanomethyl)-7-((3,3-difluoro-1-methylpiperidin-4-yl)amino)benzofuran-2-yl)prop-2-yn-1-yl)amino)- |
| | 3-methoxy-N-methylbenzamide; |
| 368 | 2-(7-((3,3-difluoro-1-methylpiperidin-4-yl)amino)-2-(3-((2-methoxy-4-(methylsulfonyl)phenyl)amino)prop- |
| | 1-yn-1-yl)benzofuran-3-yl)acetonitrile; |
| 369 | 4-((3-(3-(cyanomethyl)-7-((1,1-dioxidotetrahydro-2H-thiopyran-4-yl)amino)benzofuran-2-yl)prop-2-yn-1- |
| | yl)amino)-3-methoxybenzenesulfonamide; |
| 370 | 2-(7-((1,1-dioxidotetrahydro-2H-thiopyran-4-yl)amino)-2-(3-((5-fluoro-2-methoxy-4-(methylsulfonyl)phenyl) |
| | amino)prop-1-yn-1-yl)benzofuran-3-yl)acetonitrile; |
| 371 | 4-((3-(3-(cyanomethyl)-7-((1,1-dioxidotetrahydro-2H-thiopyran-4-yl)amino)benzofuran-2-yl)prop-2-yn-1- |
| | yl)amino)-3-methoxy-N-methylbenzamide; |
| 372 | 2-(7-((1,1-dioxidotetrahydro-2H-thiopyran-4-yl)amino)-2-(3-((2-methoxy-4-(methylsulfonyl)phenyl)amino) |
| | prop-1-yn-1-yl)benzofuran-3-yl)acetonitrile; |
| 373 | 4-((3-(3-(cyanomethyl)-7-(((3R,4S)-3-fluoro-1-methylpiperidin-4-yl)amino)benzofuran-2-yl)prop-2-yn-1- |
| | yl)amino)-3-methoxybenzenesulfonamide; |
| 374 | 2-(7-(((3R,4S)-3-fluoro-1-methylpiperidin-4-yl)amino)-2-(3-((5-fluoro-2-methoxy-4-(methylsulfonyl)phenyl) |
| | amino)prop-1-yn-1-yl)benzofuran-3-yl)acetonitrile; |
| 375 | 4-((3-(3-(cyanomethyl)-7-(((3R,4S)-3-fluoro-1-methylpiperidin-4-yl)amino)benzofuran-2-yl)prop-2-yn-1- |
| | yl)amino)-3-methoxy-N-methylbenzamide; |
| 376 | 2-(7-(((3R,4S)-3-fluoro-1-methylpiperidin-4-yl)amino)-2-(3-((2-methoxy-4-(methylsulfonyl)phenyl)amino) |
| | prop-1-yn-1-yl)benzofuran-3-yl)acetonitrile; |
| 377 | 4-((3-(3-(cyanomethyl)-7-((3-fluoro-1-methylpiperidin-4-yl)amino)benzofuran-2-yl)prop-2-yn-1-yl)amino)- |
| | 3-methoxybenzenesulfonamide; |
| 378 | 2-(7-((3-fluoro-1-methylpiperidin-4-yl)amino)-2-(3-((5-fluoro-2-methoxy-4-(methylsulfonyl)phenyl)amino) |
| | prop-1-yn-1-yl)benzofuran-3-yl)acetonitrile; |
| 379 | 4-((3-(3-(cyanomethyl)-7-((3-fluoro-1-methylpiperidin-4-yl)amino)benzofuran-2-yl)prop-2-yn-1-yl)amino)- |
| | 3-methoxy-N-methylbenzamide; |
| 380 | 2-(7-((3-fluoro-1-methylpiperidin-4-yl)amino)-2-(3-((2-methoxy-4-(methylsulfonyl)phenyl)amino)prop-1-yn- |
| | 1-yl)benzofuran-3-yl)acetonitrile; |
| 381 | 4-((3-(3-(cyanomethyl)-7-((1-(dimethylglycyl)piperidin-4-yl)amino)benzofuran-2-yl)prop-2-yn-1-yl)amino)- |
| | 3-methoxybenzenesulfonamide; |
| 382 | 2-(7-((1-(dimethylglycyl)piperidin-4-yl)amino)-2-(3-((5-fluoro-2-methoxy-4-(methylsulfonyl)phenyl)amino) |
| | prop-1-yn-1-yl)benzofuran-3-yl)acetonitrile; |
| 383 | 4-((3-(3-(cyanomethyl)-7-((1-(dimethylglycyl)piperidin-4-yl)amino)benzofuran-2-yl)prop-2-yn-1-yl)amino)- |
| | 3-methoxy-N-methylbenzamide; |
| 384 | 2-(7-((1-(dimethylglycyl)piperidin-4-yl)amino)-2-(3-((2-methoxy-4-(methylsulfonyl)phenyl)amino)prop-1- |
| | yn-1-yl)benzofuran-3-yl)acetonitrile; |
| 385 | 4-((3-(3-(cyanomethyl)-7-((3-fluoro-1-(2-hydroxy-3-methoxypropyl)piperidin-4-yl)amino)benzofuran-2-yl) |
| | prop-2-yn-1-yl)amino)-3-methoxybenzenesulfonamide; |
| 386 | 2-(7-((3-fluoro-1-(2-hydroxy-3-methoxypropyl)piperidin-4-yl)amino)-2-(3-((5-fluoro-2-methoxy-4-(methyl- |
| | sulfonyl)phenyl)amino)prop-1-yn-1-yl)benzofuran-3-yl)acetonitrile; |
| 387 | 4-((3-(3-(cyanomethyl)-7-((3-fluoro-1-(2-hydroxy-3-methoxypropyl)piperidin-4-yl)amino)benzofuran-2-yl) |
| | prop-2-yn-1-yl)amino)-3-methoxy-N-methylbenzamide; |
| 388 | 2-(7-((3-fluoro-1-(2-hydroxy-3-methoxypropyl)piperidin-4-yl)amino)-2-(3-((2-methoxy-4-(methylsulfonyl) |
| | phenyl)amino)prop-1-yn-1-yl)benzofuran-3-yl)acetonitrile; |
| 389 | 4-((3-(3-(cyanomethyl)-7-(((3R,4R)-3-fluoro-1-methylpiperidin-4-yl)amino)benzofuran-2-yl)prop-2-yn-1- |
| | yl)amino)-3-methoxybenzenesulfonamide; |
| 390 | 2-(7-(((3R,4R)-3-fluoro-1-methylpiperidin-4-yl)amino)-2-(3-((5-fluoro-2-methoxy-4-(methylsulfonyl)phenyl) |
| | amino)prop-1-yn-1-yl)benzofuran-3-yl)acetonitrile; |
| 391 | 4-((3-(3-(cyanomethyl)-7-(((3R,4R)-3-fluoro-1-methylpiperidin-4-yl)amino)benzofuran-2-yl)prop-2-yn-1- |
| | yl)amino)-3-methoxy-N-methylbenzamide; |
| 392 | 2-(7-(((3R,4R)-3-fluoro-1-methylpiperidin-4-yl)amino)-2-(3-((2-methoxy-4-(methylsulfonyl)phenyl)amino) |
| | prop-1-yn-1-yl)benzofuran-3-yl)acetonitrile; |
| 393 | 4-((3-(3-(cyanomethyl)-7-(((3S,4S)-3-fluoro-1-methylpiperidin-4-yl)amino)benzofuran-2-yl)prop-2-yn-1-yl) |
| | amino)-3-methoxybenzenesulfonamide; |
| 394 | 2-(7-(((3S,4S)-3-fluoro-1-methylpiperidin-4-yl)amino)-2-(3-((5-fluoro-2-methoxy-4-(methylsulfonyl)phenyl) |
| | amino)prop-1-yn-1-yl)benzofuran-3-yl)acetonitrile; |
| 395 | 4-((3-(3-(cyanomethyl)-7-(((3S,4S)-3-fluoro-1-methylpiperidin-4-yl)amino)benzofuran-2-yl)prop-2-yn-1- |
| | yl)amino)-3-methoxy-N-methylbenzamide; |
| 396 | 2-(7-(((3S,4S)-3-fluoro-1-methylpiperidin-4-yl)amino)-2-(3-((2-methoxy-4-(methylsulfonyl)phenyl)amino) |
| | prop-1-yn-1-yl)benzofuran-3-yl)acetonitrile; |
| 397 | 4-((3-(3-(cyanomethyl)-7-((4-(dimethylamino)cyclohexyl)amino)benzofuran-2-yl)prop-2-yn-1-yl)amino)-3- |
| | methoxybenzenesulfonamide; |
| 398 | 2-(7-((4-(dimethylamino)cyclohexyl)amino)-2-(3-((5-fluoro-2-methoxy-4-(methylsulfonyl)phenyl)amino)prop- |
| | 1-yn-1-yl)benzofuran-3-yl)acetonitrile; |
| 399 | 4-((3-(3-(cyanomethyl)-7-((4-(dimethylamino)cyclohexyl)amino)benzofuran-2-yl)prop-2-yn-1-yl)amino)-3- |
| | methoxy-N-methylbenzamide; |
| 400 | 2-(7-((4-(dimethylamino)cyclohexyl)amino)-2-(3-((2-methoxy-4-(methylsulfonyl)phenyl)amino)prop-1-yn- |
| | 1-yl)benzofuran-3-yl)acetonitrile; |
| 401 | 4-((3-(3-(cyanomethyl)-7-((2-(dimethylamino)ethyl)amino)benzofuran-2-yl)prop-2-yn-1-yl)amino)-3- |
| | methoxybenzenesulfonamide; |
| 402 | 2-(7-((2-(dimethylamino)ethyl)amino)-2-(3-((5-fluoro-2-methoxy-4-(methylsulfonyl)phenyl)amino)prop-1- |
| | yn-1-yl)benzofuran-3-yl)acetonitrile; |
| 403 | 4-((3-(3-(cyanomethyl)-7-((2-(dimethylamino)ethyl)amino)benzofuran-2-yl)prop-2-yn-1-yl)amino)-3-methoxy- |
| | N-methylbenzamide; |
| 404 | 2-(7-((2-(dimethylamino)ethyl)amino)-2-(3-((2-methoxy-4-(methylsulfonyl)phenyl)amino)prop-1-yn-1-yl)benzo- |
| | furan-3-yl)acetonitrile; |
| 405 | 4-((3-(3-(cyanomethyl)-7-(((3S,4R)-3-fluoro-1-methylpiperidin-4-yl)amino)benzofuran-2-yl)prop-2-yn-1- |
| | yl)amino)-3-methoxybenzenesulfonamide; |
| 406 | 2-(7-(((3S,4R)-3-fluoro-1-methylpiperidin-4-yl)amino)-2-(3-((5-fluoro-2-methoxy-4-(methylsulfonyl)phenyl) |
| | amino)prop-1-yn-1-yl)benzofuran-3-yl)acetonitrile; |
| 407 | 4-((3-(3-(cyanomethyl)-7-(((3S,4R)-3-fluoro-1-methylpiperidin-4-yl)amino)benzofuran-2-yl)prop-2-yn-1- |
| | yl)amino)-3-methoxy-N-methylbenzamide; |
| 408 | 2-(7-(((3S,4R)-3-fluoro-1-methylpiperidin-4-yl)amino)-2-(3-((2-methoxy-4-(methylsulfonyl)phenyl)amino) |
| | prop-1-yn-1-yl)benzofuran-3-yl)acetonitrile; |
| 409 | 4-((3-(3-(cyanomethyl)-7-((1-methylpiperidin-4-yl)amino)benzofuran-2-yl)prop-2-yn-1-yl)amino)-3-methoxy- |
| | benzenesulfonamide; |
| 410 | 2-(2-(3-((5-fluoro-2-methoxy-4-(methylsulfonyl)phenyl)amino)prop-1-yn-1-yl)-7-((1-methylpiperidin-4-yl) |
| | amino)benzofuran-3-yl)acetonitrile; |
| 411 | 4-((3-(3-(cyanomethyl)-7-((1-methylpiperidin-4-yl)amino)benzofuran-2-yl)prop-2-yn-1-yl)amino)-3-methoxy- |
| | N-methylbenzamide; |
| 412 | 2-(2-(3-((2-methoxy-4-(methylsulfonyl)phenyl)amino)prop-1-yn-1-yl)-7-((1-methylpiperidin-4-yl)amino)benzo- |
| | furan-3-yl)acetonitrile; |
| 413 | 4-((3-(3-(cyanomethyl)-7-((tetrahydro-2H-pyran-4-yl)amino)benzofuran-2-yl)prop-2-yn-1-yl)amino)-3-methoxy- |
| | benzenesulfonamide; |
| 414 | 2-(2-(3-((5-fluoro-2-methoxy-4-(methylsulfonyl)phenyl)amino)prop-1-yn-1-yl)-7-((tetrahydro-2H-pyran-4- |
| | yl)amino)benzofuran-3-yl)acetonitrile; |
| 415 | 4-((3-(3-(cyanomethyl)-7-((tetrahydro-2H-pyran-4-yl)amino)benzofuran-2-yl)prop-2-yn-1-yl)amino)-3-methoxy- |
| | N-methylbenzamide; |
| 416 | 2-(2-(3-((2-methoxy-4-(methylsulfonyl)phenyl)amino)prop-1-yn-1-yl)-7-((tetrahydro-2H-pyran-4-yl)amino) |
| | benzofuran-3-yl)acetonitrile; |
| 417 | 4-((3-(7-((3,3-difluoro-1-methylpiperidin-4-yl)amino)-3-(thiazol-4-yl)benzofuran-2-yl)prop-2-yn-1-yl)amino)- |
| | 3-methoxybenzenesulfonamide; |
| 418 | 3,3-difluoro-N-(2-(3-((5-fluoro-2-methoxy-4-(methylsulfonyl)phenyl)amino)prop-1-yn-1-yl)-3-(thiazol-4-yl) |
| | benzofuran-7-yl)-1-methylpiperidin-4-amine; |
| 419 | 4-((3-(7-((3,3-difluoro-1-methylpiperidin-4-yl)amino)-3-(thiazol-4-yl)benzofuran-2-yl)prop-2-yn-1-yl)amino)- |
| | 3-methoxy-N-methylbenzamide; |
| 420 | 3,3-difluoro-N-(2-(3-((2-methoxy-4-(methylsulfonyl)phenyl)amino)prop-1-yn-1-yl)-3-(thiazol-4-yl)benzofuran- |
| | 7-yl)-1-methylpiperidin-4-amine; |
| 421 | 4-((3-(7-((1,1-dioxidotetrahydro-2H-thiopyran-4-yl)amino)-3-(thiazol-4-yl)benzofuran-2-yl)prop-2-yn-1-yl) |
| | amino)-3-methoxybenzenesulfonamide; |
| 422 | 4-((2-(3-((5-fluoro-2-methoxy-4-(methylsulfonyl)phenyl)amino)prop-1-yn-1-yl)-3-(thiazol-4-yl)benzofuran- |
| | 7-yl)amino)tetrahydro-2H-thiopyran 1,1-dioxide; |
| 423 | 4-((3-(7-((1,1-dioxidotetrahydro-2H-thiopyran-4-yl)amino)-3-(thiazol-4-yl)benzofuran-2-yl)prop-2-yn-1-yl) |
| | amino)-3-methoxy-N-methylbenzamide; |
| 424 | 4-((2-(3-((2-methoxy-4-(methylsulfonyl)phenyl)amino)prop-1-yn-1-yl)-3-(thiazol-4-yl)benzofuran-7-yl)amino) |
| | tetrahydro-2H-thiopyran 1,1-dioxide; |
| 425 | 4-((3-(7-(((3R,4S)-3-fluoro-1-methylpiperidin-4-yl)amino)-3-(thiazol-4-yl)benzofuran-2-yl)prop-2-yn-1-yl) |
| | amino)-3-methoxybenzenesulfonamide; |
| 426 | (3R,4S)-3-fluoro-N-(2-(3-((5-fluoro-2-methoxy-4-(methylsulfonyl)phenyl)amino)prop-1-yn-1-yl)-3-(thiazol- |
| | 4-yl)benzofuran-7-yl)-1-methylpiperidin-4-amine; |
| 427 | 4-((3-(7-(((3R,4S)-3-fluoro-1-methylpiperidin-4-yl)amino)-3-(thiazol-4-yl)benzofuran-2-yl)prop-2-yn-1-yl) |
| | amino)-3-methoxy-N-methylbenzamide; |
| 428 | (3R,4S)-3-fluoro-N-(2-(3-((2-methoxy-4-(methylsulfonyl)phenyl)amino)prop-1-yn-1-yl)-3-(thiazol-4-yl)benzofuran- |
| | 7-yl)-1-methylpiperidin-4-amine; |
| 429 | 4-((3-(7-((3-fluoro-1-methylpiperidin-4-yl)amino)-3-(thiazol-4-yl)benzofuran-2-yl)prop-2-yn-1-yl)amino)-3- |
| | methoxybenzenesulfonamide; |
| 430 | 3-fluoro-N-(2-(3-((5-fluoro-2-methoxy-4-(methylsulfonyl)phenyl)amino)prop-1-yn-1-yl)-3-(thiazol-4-yl)benzofuran- |
| | 7-yl)-1-methylpiperidin-4-amine; |
| 431 | 4-((3-(7-((3-fluoro-1-methylpiperidin-4-yl)amino)-3-(thiazol-4-yl)benzofuran-2-yl)prop-2-yn-1-yl)amino)-3- |
| | methoxy-N-methylbenzamide; |
| 432 | 3-fluoro-N-(2-(3-((2-methoxy-4-(methylsulfonyl)phenyl)amino)prop-1-yn-1-yl)-3-(thiazol-4-yl)benzofuran- |
| | 7-yl)-1-methylpiperidin-4-amine; |
| 433 | 4-((3-(7-((1-(dimethylglycyl)piperidin-4-yl)amino)-3-(thiazol-4-yl)benzofuran-2-yl)prop-2-yn-1-yl)amino)- |
| | 3-methoxybenzenesulfonamide; |
| 434 | 2-(dimethylamino)-1-(4-((2-(3-((5-fluoro-2-methoxy-4-(methylsulfonyl)phenyl)amino)prop-1-yn-1-yl)-3- |
| | (thiazol-4-yl)benzofuran-7-yl)amino)piperidin-1-yl)ethan-1-one; |
| 435 | 4-((3-(7-((1-(dimethylglycyl)piperidin-4-yl)amino)-3-(thiazol-4-yl)benzofuran-2-yl)prop-2-yn-1-yl)amino)- |
| | 3-methoxy-N-methylbenzamide; |
| 436 | 2-(dimethylamino)-1-(4-((2-(3-((2-methoxy-4-(methylsulfonyl)phenyl)amino)prop-1-yn-1-yl)-3-(thiazol-4- |
| | yl)benzofuran-7-yl)amino)piperidin-1-yl)ethan-1-one; |
| 437 | 4-((3-(7-((3-fluoro-1-(2-hydroxy-3-methoxypropyl)piperidin-4-yl)amino)-3-(thiazol-4-yl)benzofuran-2-yl)prop- |
| | 2-yn-1-yl)amino)-3-methoxybenzenesulfonamide; |
| 438 | 1-(3-fluoro-4-((2-(3-((5-fluoro-2-methoxy-4-(methylsulfonyl)phenyl)amino)prop-1-yn-1-yl)-3-(thiazol-4- |
| | yl)benzofuran-7-yl)amino)piperidin-1-yl)-3-methoxypropan-2-ol; |
| 439 | 4-((3-(7-((3-fluoro-1-(2-hydroxy-3-methoxypropyl)piperidin-4-yl)amino)-3-(thiazol-4-yl)benzofuran-2-yl)prop- |
| | 2-yn-1-yl)amino)-3-methoxy-N-methylbenzamide; |
| 440 | 1-(3-fluoro-4-((2-(3-((2-methoxy-4-(methylsulfonyl)phenyl)amino)prop-1-yn-1-yl)-3-(thiazol-4-yl)benzofuran- |
| | 7-yl)amino)piperidin-1-yl)-3-methoxypropan-2-ol; |
| 441 | 4-((3-(7-(((3R,4R)-3-fluoro-1-methylpiperidin-4-yl)amino)-3-(thiazol-4-yl)benzofuran-2-yl)prop-2-yn-1-yl) |
| | amino)-3-methoxybenzenesulfonamide; |
| 442 | (3R,4R)-3-fluoro-N-(2-(3-((5-fluoro-2-methoxy-4-(methylsulfonyl)phenyl)amino)prop-1-yn-1-yl)-3-(thiazol- |
| | 4-yl)benzofuran-7-yl)-1-methylpiperidin-4-amine; |
| 443 | 4-((3-(7-(((3R,4R)-3-fluoro-1-methylpiperidin-4-yl)amino)-3-(thiazol-4-yl)benzofuran-2-yl)prop-2-yn-1-yl) |
| | amino)-3-methoxy-N-methylbenzamide; |
| 444 | (3R,4R)-3-fluoro-N-(2-(3-((2-methoxy-4-(methylsulfonyl)phenyl)amino)prop-1-yn-1-yl)-3-(thiazol-4-yl)benzofuran- |
| | 7-yl)-1-methylpiperidin-4-amine; |
| 445 | 4-((3-(7-(((3S,4S)-3-fluoro-1-methylpiperidin-4-yl)amino)-3-(thiazol-4-yl)benzofuran-2-yl)prop-2-yn-1-yl) |
| | amino)-3-methoxybenzenesulfonamide; |
| 446 | (3S,4S)-3-fluoro-N-(2-(3-((5-fluoro-2-methoxy-4-(methylsulfonyl)phenyl)amino)prop-1-yn-1-yl)-3-(thiazol- |
| | 4-yl)benzofuran-7-yl)-1-methylpiperidin-4-amine; |
| 447 | 4-((3-(7-(((3S,4S)-3-fluoro-1-methylpiperidin-4-yl)amino)-3-(thiazol-4-yl)benzofuran-2-yl)prop-2-yn-1-yl) |
| | amino)-3-methoxy-N-methylbenzamide; |
| 448 | (3S,4S)-3-fluoro-N-(2-(3-((2-methoxy-4-(methylsulfonyl)phenyl)amino)prop-1-yn-1-yl)-3-(thiazol-4-yl)benzofuran- |
| | 7-yl)-1-methylpiperidin-4-amine; |
| 449 | 4-((3-(7-((4-(dimethylamino)cyclohexyl)amino)-3-(thiazol-4-yl)benzofuran-2-yl)prop-2-yn-1-yl)amino)-3- |
| | methoxybenzenesulfonamide; |
| 450 | N1-(2-(3-((5-fluoro-2-methoxy-4-(methylsulfonyl)phenyl)amino)prop-1-yn-1-yl)-3-(thiazol-4-yl)benzofuran- |
| | 7-yl)-N4,N4-dimethylcyclohexane-1,4-diamine; |
| 451 | 4-((3-(7-((4-(dimethylamino)cyclohexyl)amino)-3-(thiazol-4-yl)benzofuran-2-yl)prop-2-yn-1-yl)amino)-3- |
| | methoxy-N-methylbenzamide; |
| 452 | N1-(2-(3-((2-methoxy-4-(methylsulfonyl)phenyl)amino)prop-1-yn-1-yl)-3-(thiazol-4-yl)benzofuran-7-yl)-N |
| | 4,N4-dimethylcyclohexane-1,4-diamine; |
| 453 | 4-((3-(7-((2-(dimethylamino)ethyl)amino)-3-(thiazol-4-yl)benzofuran-2-yl)prop-2-yn-1-yl)amino)-3- |
| | methoxybenzenesulfonamide; |
| 454 | N1-(2-(3-((5-fluoro-2-methoxy-4-(methylsulfonyl)phenyl)amino)prop-1-yn-1-yl)-3-(thiazol-4-yl)benzofuran- |
| | 7-yl)-N2,N2-dimethylethane-1,2-diamine; |
| 455 | 4-((3-(7-((2-(dimethylamino)ethyl)amino)-3-(thiazol-4-yl)benzofuran-2-yl)prop-2-yn-1-yl)amino)-3-methoxy- |
| | N-methylbenzamide; |
| 456 | N1-(2-(3-((2-methoxy-4-(methylsulfonyl)phenyl)amino)prop-1-yn-1-yl)-3-(thiazol-4-yl)benzofuran-7-yl)- |
| | N2,N2-dimethylethane-1,2-diamine; |
| 457 | 4-((3-(7-(((3S,4R)-3-fluoro-1-methylpiperidin-4-yl)amino)-3-(thiazol-4-yl)benzofuran-2-yl)prop-2-yn-1-yl) |
| | amino)-3-methoxybenzenesulfonamide; |
| 458 | (3S,4R)-3-fluoro-N-(2-(3-((5-fluoro-2-methoxy-4-(methylsulfonyl)phenyl)amino)prop-1-yn-1-yl)-3-(thiazol- |
| | 4-yl)benzofuran-7-yl)-1-methylpiperidin-4-amine; |
| 459 | 4-((3-(7-(((3S,4R)-3-fluoro-1-methylpiperidin-4-yl)amino)-3-(thiazol-4-yl)benzofuran-2-yl)prop-2-yn-1-yl) |
| | amino)-3-methoxy-N-methylbenzamide; |
| 460 | (3S,4R)-3-fluoro-N-(2-(3-((2-methoxy-4-(methylsulfonyl)phenyl)amino)prop-1-yn-1-yl)-3-(thiazol-4-yl)benzofuran- |
| | 7-yl)-1-methylpiperidin-4-amine; |
| 461 | 3-methoxy-4-((3-(7-((1-methylpiperidin-4-yl)amino)-3-(thiazol-4-yl)benzofuran-2-yl)prop-2-yn-1-yl)amino)benzene- |
| | sulfonamide; |
| 462 | N-(2-(3-((5-fluoro-2-methoxy-4-(methylsulfonyl)phenyl)amino)prop-1-yn-1-yl)-3-(thiazol-4-yl)benzofuran- |
| | 7-yl)-1-methylpiperidin-4-amine; |
| 463 | 3-methoxy-N-methyl-4-((3-(7-((1-methylpiperidin-4-yl)amino)-3-(thiazol-4-yl)benzofuran-2-yl)prop-2-yn- |
| | 1-yl)amino)benzamide; |
| 464 | N-(2-(3-((2-methoxy-4-(methylsulfonyl)phenyl)amino)prop-1-yn-1-yl)-3-(thiazol-4-yl)benzofuran-7-yl)-1- |
| | methylpiperidin-4-amine; |
| 465 | 3-methoxy-4-((3-(7-((tetrahydro-2H-pyran-4-yl)amino)-3-(thiazol-4-yl)benzofuran-2-yl)prop-2-yn-1- |
| | yl)amino)benzenesulfonamide; |
| 466 | 2-(3-((5-fluoro-2-methoxy-4-(methylsulfonyl)phenyl)amino)prop-1-yn-1-yl)-N-(tetrahydro-2H-pyran-4-yl)- |
| | 3-(thiazol-4-yl)benzofuran-7-amine; |
| 467 | 3-methoxy-N-methyl-4-((3-(7-((tetrahydro-2H-pyran-4-yl)amino)-3-(thiazol-4-yl)benzofuran-2-yl)prop-2-yn- |
| | 1-yl)amino)benzamide; |
| 468 | 2-(3-((2-methoxy-4-(methylsulfonyl)phenyl)amino)prop-1-yn-1-yl)-N-(tetrahydro-2H-pyran-4-yl)-3-(thiazol- |
| | 4-yl)benzofuran-7-amine; |
| 469 | 4-((3-(7-((3,3-difluoro-1-methylpiperidin-4-yl)amino)-3-(2,2,2-trifluoroethyl)benzofuran-2-yl)prop-2-yn-1- |
| | yl)amino)-3-methoxybenzenesulfonamide; |
| 470 | 3,3-difluoro-N-(2-(3-((5-fluoro-2-methoxy-4-(methylsulfonyl)phenyl)amino)prop-1-yn-1-yl)-3-(2,2,2- |
| | trifluoroethyl)benzofuran-7-yl)-1-methylpiperidin-4-amine; |
| 471 | 4-((3-(7-((3,3-difluoro-1-methylpiperidin-4-yl)amino)-3-(2,2,2-trifluoroethyl)benzofuran-2-yl)prop-2-yn-1- |
| | yl)amino)-3-methoxy-N-methylbenzamide; |
| 472 | 3,3-difluoro-N-(2-(3-((2-methoxy-4-(methylsulfonyl)phenyl)amino)prop-1-yn-1-yl)-3-(2,2,2-trifluoroethyl) |
| | benzofuran-7-yl)-1-methylpiperidin-4-amine; |
| 473 | 4-((3-(7-((1,1-dioxidotetrahydro-2H-thiopyran-4-yl)amino)-3-(2,2,2-trifluoroethyl)benzofuran-2-yl)prop-2- |
| | yn-1-yl)amino)-3-methoxybenzenesulfonamide; |
| 474 | 4-((2-(3-((5-fluoro-2-methoxy-4-(methylsulfonyl)phenyl)amino)prop-1-yn-1-yl)-3-(2,2,2-trifluoroethyl)benzo- |
| | furan-7-yl)amino)tetrahydro-2H-thiopyran 1,1-dioxide; |
| 475 | 4-((3-(7-((1,1-dioxidotetrahydro-2H-thiopyran-4-yl)amino)-3-(2,2,2-trifluoroethyl)benzofuran-2-yl)prop-2- |
| | yn-1-yl)amino)-3-methoxy-N-methylbenzamide; |
| 476 | 4-((2-(3-((2-methoxy-4-(methylsulfonyl)phenyl)amino)prop-1-yn-1-yl)-3-(2,2,2-trifluoroethyl)benzofuran-7- |
| | yl)amino)tetrahydro-2H-thiopyran 1,1-dioxide; |
| 477 | 4-((3-(7-(((3R,4S)-3-fluoro-1-methylpiperidin-4-yl)amino)-3-(2,2,2-trifluoroethyl)benzofuran-2-yl)prop-2-yn- |
| | 1-yl)amino)-3-methoxybenzenesulfonamide; |
| 478 | (3R,4S)-3-fluoro-N-(2-(3-((5-fluoro-2-methoxy-4-(methylsulfonyl)phenyl)amino)prop-1-yn-1-yl)-3-(2,2,2- |
| | trifluoroethyl)benzofuran-7-yl)-1-methylpiperidin-4-amine; |
| 479 | 4-((3-(7-(((3R,4S)-3-fluoro-1-methylpiperidin-4-yl)amino)-3-(2,2,2-trifluoroethyl)benzofuran-2-yl)prop-2-yn- |
| | 1-yl)amino)-3-methoxy-N-methylbenzamide; |
| 480 | (3R,4S)-3-fluoro-N-(2-(3-((2-methoxy-4-(methylsulfonyl)phenyl)amino)prop-1-yn-1-yl)-3-(2,2,2-trifluoroethyl) |
| | benzofuran-7-yl)-1-methylpiperidin-4-amine; |
| 481 | 4-((3-(7-((3-fluoro-1-methylpiperidin-4-yl)amino)-3-(2,2,2-trifluoroethyl)benzofuran-2-yl)prop-2-yn-1-yl)amino)- |
| | 3-methoxybenzenesulfonamide; |
| 482 | 3-fluoro-N-(2-(3-((5-fluoro-2-methoxy-4-(methylsulfonyl)phenyl)amino)prop-1-yn-1-yl)-3-(2,2,2- |
| | trifluoroethyl)benzofuran-7-yl)-1-methylpiperidin-4-amine; |
| 483 | 4-((3-(7-((3-fluoro-1-methylpiperidin-4-yl)amino)-3-(2,2,2-trifluoroethyl)benzofuran-2-yl)prop-2-yn-1-yl)amino)- |
| | 3-methoxy-N-methylbenzamide; |
| 484 | 3-fluoro-N-(2-(3-((2-methoxy-4-(methylsulfonyl)phenyl)amino)prop-1-yn-1-yl)-3-(2,2,2-trifluoroethyl)benzo- |
| | furan-7-yl)-1-methylpiperidin-4-amine; |
| 485 | 4-((3-(7-((1-(dimethylglycyl)piperidin-4-yl)amino)-3-(2,2,2-trifluoroethyl)benzofuran-2-yl)prop-2-yn-1-yl) |
| | amino)-3-methoxybenzenesulfonamide; |
| 486 | 2-(dimethylamino)-1-(4-((2-(3-((5-fluoro-2-methoxy-4-(methylsulfonyl)phenyl)amino)prop-1-yn-1-yl)-3-(2,2,2- |
| | trifluoroethyl)benzofuran-7-yl)amino)piperidin-1-yl)ethan-1-one; |
| 487 | 4-((3-(7-((1-(dimethylglycyl)piperidin-4-yl)amino)-3-(2,2,2-trifluoroethyl)benzofuran-2-yl)prop-2-yn-1-yl) |
| | amino)-3-methoxy-N-methylbenzamide; |
| 488 | 2-(dimethylamino)-1-(4-((2-(3-((2-methoxy-4-(methylsulfonyl)phenyl)amino)prop-1-yn-1-yl)-3-(2,2,2- |
| | trifluoroethyl)benzofuran-7-yl)amino)piperidin-1-yl)ethan-1-one; |
| 489 | 4-((3-(7-((3-fluoro-1-(2-hydroxy-3-methoxypropyl)piperidin-4-yl)amino)-3-(2,2,2-trifluoroethyl)benzofuran- |
| | 2-yl)prop-2-yn-1-yl)amino)-3-methoxybenzenesulfonamide; |
| 490 | 1-(3-fluoro-4-((2-(3-((5-fluoro-2-methoxy-4-(methylsulfonyl)phenyl)amino)prop-1-yn-1-yl)-3-(2,2,2- |
| | trifluoroethyl)benzofuran-7-yl)amino)piperidin-1-yl)-3-methoxypropan-2-ol; |
| 491 | 4-((3-(7-((3-fluoro-1-(2-hydroxy-3-methoxypropyl)piperidin-4-yl)amino)-3-(2,2,2-trifluoroethyl)benzofuran- |
| | 2-yl)prop-2-yn-1-yl)amino)-3-methoxy-N-methylbenzamide; |
| 492 | 1-(3-fluoro-4-((2-(3-((2-methoxy-4-(methylsulfonyl)phenyl)amino)prop-1-yn-1-yl)-3-(2,2,2-trifluoroethyl) |
| | benzofuran-7-yl)amino)piperidin-1-yl)-3-methoxypropan-2-ol; |
| 493 | 4-((3-(7-(((3R,4R)-3-fluoro-1-methylpiperidin-4-yl)amino)-3-(2,2,2-trifluoroethyl)benzofuran-2-yl)prop-2- |
| | yn-1-yl)amino)-3-methoxybenzenesulfonamide; |
| 494 | (3R,4R)-3-fluoro-N-(2-(3-((5-fluoro-2-methoxy-4-(methylsulfonyl)phenyl)amino)prop-1-yn-1-yl)-3-(2,2,2- |
| | trifluoroethyl)benzofuran-7-yl)-1-methylpiperidin-4-amine; |
| 495 | 4-((3-(7-(((3R,4R)-3-fluoro-1-methylpiperidin-4-yl)amino)-3-(2,2,2-trifluoroethyl)benzofuran-2-yl)prop-2- |
| | yn-1-yl)amino)-3-methoxy-N-methylbenzamide; |
| 496 | (3R,4R)-3-fluoro-N-(2-(3-((2-methoxy-4-(methylsulfonyl)phenyl)amino)prop-1-yn-1-yl)-3-(2,2,2- |
| | trifluoroethyl)benzofuran-7-yl)-1-methylpiperidin-4-amine; |
| 497 | 4-((3-(7-(((3S,4S)-3-fluoro-1-methylpiperidin-4-yl)amino)-3-(2,2,2-trifluoroethyl)benzofuran-2-yl)prop-2-yn- |
| | 1-yl)amino)-3-methoxybenzenesulfonamide; |
| 498 | (3S,4S)-3-fluoro-N-(2-(3-((5-fluoro-2-methoxy-4-(methylsulfonyl)phenyl)amino)prop-1-yn-1-yl)-3-(2,2,2- |
| | trifluoroethyl)benzofuran-7-yl)-1-methylpiperidin-4-amine; |
| 499 | 4-((3-(7-(((3S,4S)-3-fluoro-1-methylpiperidin-4-yl)amino)-3-(2,2,2-trifluoroethyl)benzofuran-2-yl)prop-2-yn- |
| | 1-yl)amino)-3-methoxy-N-methylbenzamide; |
| 500 | (3S,4S)-3-fluoro-N-(2-(3-((2-methoxy-4-(methylsulfonyl)phenyl)amino)prop-1-yn-1-yl)-3-(2,2,2-trifluoroethyl) |
| | benzofuran-7-yl)-1-methylpiperidin-4-amine; |
| 501 | 4-((3-(7-((4-(dimethylamino)cyclohexyl)amino)-3-(2,2,2-trifluoroethyl)benzofuran-2-yl)prop-2-yn-1-yl)amino)- |
| | 3-methoxybenzenesulfonamide; |
| 502 | N1-(2-(3-((5-fluoro-2-methoxy-4-(methylsulfonyl)phenyl)amino)prop-1-yn-1-yl)-3-(2,2,2-trifluoroethyl)benzofuran- |
| | 7-yl)-N4,N4-dimethylcyclohexane-1,4-diamine; |
| 503 | 4-((3-(7-((4-(dimethylamino)cyclohexyl)amino)-3-(2,2,2-trifluoroethyl)benzofuran-2-yl)prop-2-yn-1-yl)amino)- |
| | 3-methoxy-N-methylbenzamide; |
| 504 | N1-(2-(3-((2-methoxy-4-(methylsulfonyl)phenyl)amino)prop-1-yn-1-yl)-3-(2,2,2-trifluoroethyl)benzofuran- |
| | 7-yl)-N4,N4-dimethylcyclohexane-1,4-diamine; |
| 505 | 4-((3-(7-((2-(dimethylamino)ethyl)amino)-3-(2,2,2-trifluoroethyl)benzofuran-2-yl)prop-2-yn-1-yl)amino)-3- |
| | methoxybenzenesulfonamide; |
| 506 | N1-(2-(3-((5-fluoro-2-methoxy-4-(methylsulfonyl)phenyl)amino)prop-1-yn-1-yl)-3-(2,2,2-trifluoroethyl)benzofuran- |
| | 7-yl)-N2,N2-dimethylethane-1,2-diamine; |
| 507 | 4-((3-(7-((2-(dimethylamino)ethyl)amino)-3-(2,2,2-trifluoroethyl)benzofuran-2-yl)prop-2-yn-1-yl)amino)-3- |
| | methoxy-N-methylbenzamide; |
| 508 | N1-(2-(3-((2-methoxy-4-(methylsulfonyl)phenyl)amino)prop-1-yn-1-yl)-3-(2,2,2-trifluoroethyl)benzofuran- |
| | 7-yl)-N2,N2-dimethylethane-1,2-diamine; |
| 509 | 4-((3-(7-(((3S,4R)-3-fluoro-1-methylpiperidin-4-yl)amino)-3-(2,2,2-trifluoroethyl)benzofuran-2-yl)prop-2-yn- |
| | 1-yl)amino)-3-methoxybenzenesulfonamide; |
| 510 | (3S,4R)-3-fluoro-N-(2-(3-((5-fluoro-2-methoxy-4-(methylsulfonyl)phenyl)amino)prop-1-yn-1-yl)-3-(2,2,2- |
| | trifluoroethyl)benzofuran-7-yl)-1-methylpiperidin-4-amine; |
| 511 | 4-((3-(7-(((3S,4R)-3-fluoro-1-methylpiperidin-4-yl)amino)-3-(2,2,2-trifluoroethyl)benzofuran-2-yl)prop-2-yn- |
| | 1-yl)amino)-3-methoxy-N-methylbenzamide; |
| 512 | (3S,4R)-3-fluoro-N-(2-(3-((2-methoxy-4-(methylsulfonyl)phenyl)amino)prop-1-yn-1-yl)-3-(2,2,2- |
| | trifluoroethyl)benzofuran-7-yl)-1-methylpiperidin-4-amine; |
| 513 | 3-methoxy-4-((3-(7-((1-methylpiperidin-4-yl)amino)-3-(2,2,2-trifluoroethyl)benzofuran-2-yl)prop-2-yn-1- |
| | yl)amino)benzenesulfonamide; |
| 514 | N-(2-(3-((5-fluoro-2-methoxy-4-(methylsulfonyl)phenyl)amino)prop-1-yn-1-yl)-3-(2,2,2-trifluoroethyl)benzofuran- |
| | 7-yl)-1-methylpiperidin-4-amine; |
| 515 | 3-methoxy-N-methyl-4-((3-(7-((1-methylpiperidin-4-yl)amino)-3-(2,2,2-trifluoroethyl)benzofuran-2-yl)prop- |
| | 2-yn-1-yl)amino)benzamide; |
| 516 | N-(2-(3-((2-methoxy-4-(methylsulfonyl)phenyl)amino)prop-1-yn-1-yl)-3-(2,2,2-trifluoroethyl)benzofuran-7- |
| | yl)-1-methylpiperidin-4-amine; |
| 517 | 3-methoxy-4-((3-(7-((tetrahydro-2H-pyran-4-yl)amino)-3-(2,2,2-trifluoroethyl)benzofuran-2-yl)prop-2-yn-1- |
| | yl)amino)benzenesulfonamide; |
| 518 | 2-(3-((5-fluoro-2-methoxy-4-(methylsulfonyl)phenyl)amino)prop-1-yn-1-yl)-N-(tetrahydro-2H-pyran-4-yl)- |
| | 3-(2,2,2-trifluoroethyl)benzofuran-7-amine; |
| 519 | 3-methoxy-N-methyl-4-((3-(7-((tetrahydro-2H-pyran-4-yl)amino)-3-(2,2,2-trifluoroethyl)benzofuran-2-yl)prop- |
| | 2-yn-1-yl)amino)benzamide; |
| 520 | 2-(3-((2-methoxy-4-(methylsulfonyl)phenyl)amino)prop-1-yn-1-yl)-N-(tetrahydro-2H-pyran-4-yl)-3-(2,2,2- |
| | trifluoroethyl)benzofuran-7-amine; |
| 521 | 4-((3-(3-(cyclopropylmethyl)-7-((3,3-difluoro-1-methylpiperidin-4-yl)amino)benzofuran-2-yl)prop-2-yn-1- |
| | yl)amino)-3-methoxybenzenesulfonamide; |
| 522 | N-(3-(cyclopropylmethyl)-2-(3-((5-fluoro-2-methoxy-4-(methylsulfonyl)phenyl)amino)prop-1-yn-1-yl)benzofuran- |
| | 7-yl)-3,3-difluoro-1-methylpiperidin-4-amine; |
| 523 | 4-((3-(3-(cyclopropylmethyl)-7-((3,3-difluoro-1-methylpiperidin-4-yl)amino)benzofuran-2-yl)prop-2-yn-1- |
| | yl)amino)-3-methoxy-N-methylbenzamide; |
| 524 | N-(3-(cyclopropylmethyl)-2-(3-((2-methoxy-4-(methylsulfonyl)phenyl)amino)prop-1-yn-1-yl)benzofuran-7- |
| | yl)-3,3-difluoro-1-methylpiperidin-4-amine; |
| 525 | 4-((3-(3-(cyclopropylmethyl)-7-((1,1-dioxidotetrahydro-2H-thiopyran-4-yl)amino)benzofuran-2-yl)prop-2- |
| | yn-1-yl)amino)-3-methoxybenzenesulfonamide; |
| 526 | 4-((3-(cyclopropylmethyl)-2-(3-((5-fluoro-2-methoxy-4-(methylsulfonyl)phenyl)amino)prop-1-yn-1-yl)benzofuran- |
| | 7-yl)amino)tetrahydro-2H-thiopyran 1,1-dioxide; |
| 527 | 4-((3-(3-(cyclopropylmethyl)-7-((1,1-dioxidotetrahydro-2H-thiopyran-4-yl)amino)benzofuran-2-yl)prop-2- |
| | yn-1-yl)amino)-3-methoxy-N-methylbenzamide; |
| 528 | 4-((3-(cyclopropylmethyl)-2-(3-((2-methoxy-4-(methylsulfonyl)phenyl)amino)prop-1-yn-1-yl)benzofuran-7- |
| | yl)amino)tetrahydro-2H-thiopyran 1,1-dioxide; |
| 529 | 4-((3-(3-(cyclopropylmethyl)-7-(((3R,4S)-3-fluoro-1-methylpiperidin-4-yl)amino)benzofuran-2-yl)prop-2-yn- |
| | 1-yl)amino)-3-methoxybenzenesulfonamide; |
| 530 | (3R,4S)-N-(3-(cyclopropylmethyl)-2-(3-((5-fluoro-2-methoxy-4-(methylsulfonyl)phenyl)amino)prop-1-yn- |
| | 1-yl)benzofuran-7-yl)-3-fluoro-1-methylpiperidin-4-amine; |
| 531 | 4-((3-(3-(cyclopropylmethyl)-7-(((3R,4S)-3-fluoro-1-methylpiperidin-4-yl)amino)benzofuran-2-yl)prop-2-yn- |
| | 1-yl)amino)-3-methoxy-N-methylbenzamide; |
| 532 | (3R,4S)-N-(3-(cyclopropylmethyl)-2-(3-((2-methoxy-4-(methylsulfonyl)phenyl)amino)prop-1-yn-1-yl)benzofuran- |
| | 7-yl)-3-fluoro-1-methylpiperidin-4-amine; |
| 533 | 4-((3-(3-(cyclopropylmethyl)-7-((3-fluoro-1-methylpiperidin-4-yl)amino)benzofuran-2-yl)prop-2-yn-1-yl)amino)- |
| | 3-methoxybenzenesulfonamide; |
| 534 | N-(3-(cyclopropylmethyl)-2-(3-((5-fluoro-2-methoxy-4-(methylsulfonyl)phenyl)amino)prop-1-yn-1-yl)benzofuran- |
| | 7-yl)-3-fluoro-1-methylpiperidin-4-amine; |
| 535 | 4-((3-(3-(cyclopropylmethyl)-7-((3-fluoro-1-methylpiperidin-4-yl)amino)benzofuran-2-yl)prop-2-yn-1-yl)amino)- |
| | 3-methoxy-N-methylbenzamide; |
| 536 | N-(3-(cyclopropylmethyl)-2-(3-((2-methoxy-4-(methylsulfonyl)phenyl)amino)prop-1-yn-1-yl)benzofuran-7- |
| | yl)-3-fluoro-1-methylpiperidin-4-amine; |
| 537 | 4-((3-(3-(cyclopropylmethyl)-7-((1-(dimethylglycyl)piperidin-4-yl)amino)benzofuran-2-yl)prop-2-yn-1-yl)amino)- |
| | 3-methoxybenzenesulfonamide; |
| 538 | 1-(4-((3-(cyclopropylmethyl)-2-(3-((5-fluoro-2-methoxy-4-(methylsulfonyl)phenyl)amino)prop-1-yn-1-yl)benzo- |
| | furan-7-yl)amino)piperidin-1-yl)-2-(dimethylamino)ethan-1-one; |
| 539 | 4-((3-(3-(cyclopropylmethyl)-7-((1-(dimethylglycyl)piperidin-4-yl)amino)benzofuran-2-yl)prop-2-yn-1-yl)amino)- |
| | 3-methoxy-N-methylbenzamide; |
| 540 | 1-(4-((3-(cyclopropylmethyl)-2-(3-((2-methoxy-4-(methylsulfonyl)phenyl)amino)prop-1-yn-1-yl)benzofuran- |
| | 7-yl)amino)piperidin-1-yl)-2-(dimethylamino)ethan-1-one; |
| 541 | 4-((3-(3-(cyclopropylmethyl)-7-((3-fluoro-1-(2-hydroxy-3-methoxypropyl)piperidin-4-yl)amino)benzofuran- |
| | 2-yl)prop-2-yn-1-yl)amino)-3-methoxybenzenesulfonamide; |
| 542 | 1-(4-((3-(cyclopropylmethyl)-2-(3-((5-fluoro-2-methoxy-4-(methylsulfonyl)phenyl)amino)prop-1-yn-1-yl)benzo- |
| | furan-7-yl)amino)-3-fluoropiperidin-1-yl)-3-methoxypropan-2-ol; |
| 543 | 4-((3-(3-(cyclopropylmethyl)-7-((3-fluoro-1-(2-hydroxy-3-methoxypropyl)piperidin-4-yl)amino)benzofuran- |
| | 2-yl)prop-2-yn-1-yl)amino)-3-methoxy-N-methylbenzamide; |
| 544 | 1-(4-((3-(cyclopropylmethyl)-2-(3-((2-methoxy-4-(methylsulfonyl)phenyl)amino)prop-1-yn-1-yl)benzofuran- |
| | 7-yl)amino)-3-fluoropiperidin-1-yl)-3-methoxypropan-2-ol; |
| 545 | 4-((3-(3-(cyclopropylmethyl)-7-(((3R,4R)-3-fluoro-1-methylpiperidin-4-yl)amino)benzofuran-2-yl)prop-2-yn- |
| | 1-yl)amino)-3-methoxybenzenesulfonamide; |
| 546 | (3R,4R)-N-(3-(cyclopropylmethyl)-2-(3-((5-fluoro-2-methoxy-4-(methylsulfonyl)phenyl)amino)prop-1-yn- |
| | 1-yl)benzofuran-7-yl)-3-fluoro-1-methylpiperidin-4-amine; |
| 547 | 4-((3-(3-(cyclopropylmethyl)-7-(((3R,4R)-3-fluoro-1-methylpiperidin-4-yl)amino)benzofuran-2-yl)prop-2-yn- |
| | 1-yl)amino)-3-methoxy-N-methylbenzamide; |
| 548 | (3R,4R)-N-(3-(cyclopropylmethyl)-2-(3-((2-methoxy-4-(methylsulfonyl)phenyl)amino)prop-1-yn-1-yl)benzo- |
| | furan-7-yl)-3-fluoro-1-methylpiperidin-4-amine; |
| 549 | 4-((3-(3-(cyclopropylmethyl)-7-(((3S,4S)-3-fluoro-1-methylpiperidin-4-yl)amino)benzofuran-2-yl)prop-2-yn- |
| | 1-yl)amino)-3-methoxybenzenesulfonamide; |
| 550 | (3S,4S)-N-(3-(cyclopropylmethyl)-2-(3-((5-fluoro-2-methoxy-4-(methylsulfonyl)phenyl)amino)prop-1-yn-1- |
| | yl)benzofuran-7-yl)-3-fluoro-1-methylpiperidin-4-amine; |
| 551 | 4-((3-(3-(cyclopropylmethyl)-7-(((3S,4S)-3-fluoro-1-methylpiperidin-4-yl)amino)benzofuran-2-yl)prop-2-yn- |
| | 1-yl)amino)-3-methoxy-N-methylbenzamide; |
| 552 | (3S,4S)-N-(3-(cyclopropylmethyl)-2-(3-((2-methoxy-4-(methylsulfonyl)phenyl)amino)prop-1-yn-1-yl)benzo- |
| | furan-7-yl)-3-fluoro-1-methylpiperidin-4-amine; |
| 553 | 4-((3-(3-(cyclopropylmethyl)-7-((4-(dimethylamino)cyclohexyl)amino)benzofuran-2-yl)prop-2-yn-1-yl)amino)- |
| | 3-methoxybenzenesulfonamide; |
| 554 | N1-(3-(cyclopropylmethyl)-2-(3-((5-fluoro-2-methoxy-4-(methylsulfonyl)phenyl)amino)prop-1-yn-1-yl)benzo- |
| | furan-7-yl)-N4,N4-dimethylcyclohexane-1,4-diamine; |
| 555 | 4-((3-(3-(cyclopropylmethyl)-7-((4-(dimethylamino)cyclohexyl)amino)benzofuran-2-yl)prop-2-yn-1-yl)amino)- |
| | 3-methoxy-N-methylbenzamide; |
| 556 | N1-(3-(cyclopropylmethyl)-2-(3-((2-methoxy-4-(methylsulfonyl)phenyl)amino)prop-1-yn-1-yl)benzofuran- |
| | 7-yl)-N4,N4-dimethylcyclohexane-1,4-diamine; |
| 557 | 4-((3-(3-(cyclopropylmethyl)-7-((2-(dimethylamino)ethyl)amino)benzofuran-2-yl)prop-2-yn-1-yl)amino)-3- |
| | methoxybenzenesulfonamide; |
| 558 | N1-(3-(cyclopropylmethyl)-2-(3-((5-fluoro-2-methoxy-4-(methylsulfonyl)phenyl)amino)prop-1-yn-1-yl)benzo- |
| | furan-7-yl)-N2,N2-dimethylethane-1,2-diamine; |
| 559 | 4-((3-(3-(cyclopropylmethyl)-7-((2-(dimethylamino)ethyl)amino)benzofuran-2-yl)prop-2-yn-1-yl)amino)-3- |
| | methoxy-N-methylbenzamide; |
| 560 | N1-(3-(cyclopropylmethyl)-2-(3-((2-methoxy-4-(methylsulfonyl)phenyl)amino)prop-1-yn-1-yl)benzofuran- |
| | 7-yl)-N2,N2-dimethylethane-1,2-diamine; |
| 561 | 4-((3-(3-(cyclopropylmethyl)-7-(((3S,4R)-3-fluoro-1-methylpiperidin-4-yl)amino)benzofuran-2-yl)prop-2-yn- |
| | 1-yl)amino)-3-methoxybenzenesulfonamide; |
| 562 | (3S,4R)-N-(3-(cyclopropylmethyl)-2-(3-((5-fluoro-2-methoxy-4-(methylsulfonyl)phenyl)amino)prop-1-yn- |
| | 1-yl)benzofuran-7-yl)-3-fluoro-1-methylpiperidin-4-amine; |
| 563 | 4-((3-(3-(cyclopropylmethyl)-7-(((3S,4R)-3-fluoro-1-methylpiperidin-4-yl)amino)benzofuran-2-yl)prop-2-yn- |
| | 1-yl)amino)-3-methoxy-N-methylbenzamide; |
| 564 | (3S,4R)-N-(3-(cyclopropylmethyl)-2-(3-((2-methoxy-4-(methylsulfonyl)phenyl)amino)prop-1-yn-1-yl)benzo- |
| | furan-7-yl)-3-fluoro-1-methylpiperidin-4-amine; |
| 565 | 4-((3-(3-(cyclopropylmethyl)-7-((1-methylpiperidin-4-yl)amino)benzofuran-2-yl)prop-2-yn-1-yl)amino)-3- |
| | methoxybenzenesulfonamide; |
| 566 | N-(3-(cyclopropylmethyl)-2-(3-((5-fluoro-2-methoxy-4-(methylsulfonyl)phenyl)amino)prop-1-yn-1-yl)benzo- |
| | furan-7-yl)-1-methylpiperidin-4-amine; |
| 567 | 4-((3-(3-(cyclopropylmethyl)-7-((1-methylpiperidin-4-yl)amino)benzofuran-2-yl)prop-2-yn-1-yl)amino)-3- |
| | methoxy-N-methylbenzamide; |
| 568 | N-(3-(cyclopropylmethyl)-2-(3-((2-methoxy-4-(methylsulfonyl)phenyl)amino)prop-1-yn-1-yl)benzofuran-7- |
| | yl)-1-methylpiperidin-4-amine; |
| 569 | 4-((3-(3-(cyclopropylmethyl)-7-((tetrahydro-2H-pyran-4-yl)amino)benzofuran-2-yl)prop-2-yn-1-yl)amino)- |
| | 3-methoxybenzenesulfonamide; |
| 570 | 3-(cyclopropylmethyl)-2-(3-((5-fluoro-2-methoxy-4-(methylsulfonyl)phenyl)amino)prop-1-yn-1-yl)-N-(tetrahydro- |
| | 2H-pyran-4-yl)benzofuran-7-amine; |
| 571 | 4-((3-(3-(cyclopropylmethyl)-7-((tetrahydro-2H-pyran-4-yl)amino)benzofuran-2-yl)prop-2-yn-1-yl)amino)- |
| | 3-methoxy-N-methylbenzamide; |
| 572 | 3-(cyclopropylmethyl)-2-(3-((2-methoxy-4-(methylsulfonyl)phenyl)amino)prop-1-yn-1-yl)-N-(tetrahydro-2H- |
| | pyran-4-yl)benzofuran-7-amine; |
| 573 | 4-((3-(7-((3,3-difluoro-1-methylpiperidin-4-yl)amino)-3-(2,2-difluoroethyl)benzofuran-2-yl)prop-2-yn-1-yl) |
| | amino)-3-methoxybenzenesulfonamide; |
| 574 | N-(3-(2,2-difluoroethyl)-2-(3-((5-fluoro-2-methoxy-4-(methylsulfonyl)phenyl)amino)prop-1-yn-1-yl)benzo- |
| | furan-7-yl)-3,3-difluoro-1-methylpiperidin-4-amine; |
| 575 | 4-((3-(7-((3,3-difluoro-1-methylpiperidin-4-yl)amino)-3-(2,2-difluoroethyl)benzofuran-2-yl)prop-2-yn-1-yl) |
| | amino)-3-methoxy-N-methylbenzamide; |
| 576 | N-(3-(2,2-difluoroethyl)-2-(3-((2-methoxy-4-(methylsulfonyl)phenyl)amino)prop-1-yn-1-yl)benzofuran-7-yl)- |
| | 3,3-difluoro-1-methylpiperidin-4-amine; |
| 577 | 4-((3-(3-(2,2-difluoroethyl)-7-((1,1-dioxidotetrahydro-2H-thiopyran-4-yl)amino)benzofuran-2-yl)prop-2-yn- |
| | 1-yl)amino)-3-methoxybenzenesulfonamide; |
| 578 | 4-((3-(2,2-difluoroethyl)-2-(3-((5-fluoro-2-methoxy-4-(methylsulfonyl)phenyl)amino)prop-1-yn-1-yl)benzo- |
| | furan-7-yl)amino)tetrahydro-2H-thiopyran 1,1-dioxide; |
| 579 | 4-((3-(3-(2,2-difluoroethyl)-7-((1,1-dioxidotetrahydro-2H-thiopyran-4-yl)amino)benzofuran-2-yl)prop-2-yn- |
| | 1-yl)amino)-3-methoxy-N-methylbenzamide; |
| 580 | 4-((3-(2,2-difluoroethyl)-2-(3-((2-methoxy-4-(methylsulfonyl)phenyl)amino)prop-1-yn-1-yl)benzofuran-7- |
| | yl)amino)tetrahydro-2H-thiopyran 1,1-dioxide; |
| 581 | 4-((3-(3-(2,2-difluoroethyl)-7-(((3R,4S)-3-fluoro-1-methylpiperidin-4-yl)amino)benzofuran-2-yl)prop-2-yn- |
| | 1-yl)amino)-3-methoxybenzenesulfonamide; |
| 582 | (3R,4S)-N-(3-(2,2-difluoroethyl)-2-(3-((5-fluoro-2-methoxy-4-(methylsulfonyl)phenyl)amino)prop-1-yn-1- |
| | yl)benzofuran-7-yl)-3-fluoro-1-methylpiperidin-4-amine; |
| 583 | 4-((3-(3-(2,2-difluoroethyl)-7-(((3R,4S)-3-fluoro-1-methylpiperidin-4-yl)amino)benzofuran-2-yl)prop-2-yn- |
| | 1-yl)amino)-3-methoxy-N-methylbenzamide; |
| 584 | (3R,4S)-N-(3-(2,2-difluoroethyl)-2-(3-((2-methoxy-4-(methylsulfonyl)phenyl)amino)prop-1-yn-1-yl)benzofuran- |
| | 7-yl)-3-fluoro-1-methylpiperidin-4-amine; |
| 585 | 4-((3-(3-(2,2-difluoroethyl)-7-((3-fluoro-1-methylpiperidin-4-yl)amino)benzofuran-2-yl)prop-2-yn-1-yl)amino)- |
| | 3-methoxybenzenesulfonamide; |
| 586 | N-(3-(2,2-difluoroethyl)-2-(3-((5-fluoro-2-methoxy-4-(methylsulfonyl)phenyl)amino)prop-1-yn-1-yl)benzofuran- |
| | 7-yl)-3-fluoro-1-methylpiperidin-4-amine; |
| 587 | 4-((3-(3-(2,2-difluoroethyl)-7-((3-fluoro-1-methylpiperidin-4-yl)amino)benzofuran-2-yl)prop-2-yn-1-yl)amino)- |
| | 3-methoxy-N-methylbenzamide; |
| 588 | N-(3-(2,2-difluoroethyl)-2-(3-((2-methoxy-4-(methylsulfonyl)phenyl)amino)prop-1-yn-1-yl)benzofuran-7-yl)- |
| | 3-fluoro-1-methylpiperidin-4-amine; |
| 589 | 4-((3-(3-(2,2-difluoroethyl)-7-((1-(dimethylglycyl)piperidin-4-yl)amino)benzofuran-2-yl)prop-2-yn-1-yl)amino)- |
| | 3-methoxybenzenesulfonamide; |
| 590 | 1-(4-((3-(2,2-difluoroethyl)-2-(3-((5-fluoro-2-methoxy-4-(methylsulfonyl)phenyl)amino)prop-1-yn-1-yl)benzo- |
| | furan-7-yl)amino)piperidin-1-yl)-2-(dimethylamino)ethan-1-one; |
| 591 | 4-((3-(3-(2,2-difluoroethyl)-7-((1-(dimethylglycyl)piperidin-4-yl)amino)benzofuran-2-yl)prop-2-yn-1-yl)amino)- |
| | 3-methoxy-N-methylbenzamide; |
| 592 | 1-(4-((3-(2,2-difluoroethyl)-2-(3-((2-methoxy-4-(methylsulfonyl)phenyl)amino)prop-1-yn-1-yl)benzofuran- |
| | 7-yl)amino)piperidin-1-yl)-2-(dimethylamino)ethan-1-one; |
| 593 | 4-((3-(3-(2,2-difluoroethyl)-7-((3-fluoro-1-(2-hydroxy-3-methoxypropyl)piperidin-4-yl)amino)benzofuran- |
| | 2-yl)prop-2-yn-1-yl)amino)-3-methoxybenzenesulfonamide; |
| 594 | 1-(4-((3-(2,2-difluoroethyl)-2-(3-((5-fluoro-2-methoxy-4-(methylsulfonyl)phenyl)amino)prop-1-yn-1-yl)benzo- |
| | furan-7-yl)amino)-3-fluoropiperidin-1-yl)-3-methoxypropan-2-ol; |
| 595 | 4-((3-(3-(2,2-difluoroethyl)-7-((3-fluoro-1-(2-hydroxy-3-methoxypropyl)piperidin-4-yl)amino)benzofuran- |
| | 2-yl)prop-2-yn-1-yl)amino)-3-methoxy-N-methylbenzamide; |
| 596 | 1-(4-((3-(2,2-difluoroethyl)-2-(3-((2-methoxy-4-(methylsulfonyl)phenyl)amino)prop-1-yn-1-yl)benzofuran- |
| | 7-yl)amino)-3-fluoropiperidin-1-yl)-3-methoxypropan-2-ol; |
| 597 | 4-((3-(3-(2,2-difluoroethyl)-7-(((3R,4R)-3-fluoro-1-methylpiperidin-4-yl)amino)benzofuran-2-yl)prop-2-yn- |
| | 1-yl)amino)-3-methoxybenzenesulfonamide; |
| 598 | (3R,4R)-N-(3-(2,2-difluoroethyl)-2-(3-((5-fluoro-2-methoxy-4-(methylsulfonyl)phenyl)amino)prop-1-yn-1- |
| | yl)benzofuran-7-yl)-3-fluoro-1-methylpiperidin-4-amine; |
| 599 | 4-((3-(3-(2,2-difluoroethyl)-7-(((3R,4R)-3-fluoro-1-methylpiperidin-4-yl)amino)benzofuran-2-yl)prop-2-yn- |
| | 1-yl)amino)-3-methoxy-N-methylbenzamide; |
| 600 | (3R,4R)-N-(3-(2,2-difluoroethyl)-2-(3-((2-methoxy-4-(methylsulfonyl)phenyl)amino)prop-1-yn-1-yl)benzo- |
| | furan-7-yl)-3-fluoro-1-methylpiperidin-4-amine; |
| 601 | 4-((3-(3-(2,2-difluoroethyl)-7-(((3S,4S)-3-fluoro-1-methylpiperidin-4-yl)amino)benzofuran-2-yl)prop-2-yn- |
| | 1-yl)amino)-3-methoxybenzenesulfonamide; |
| 602 | (3S,4S)-N-(3-(2,2-difluoroethyl)-2-(3-((5-fluoro-2-methoxy-4-(methylsulfonyl)phenyl)amino)prop-1-yn-1- |
| | yl)benzofuran-7-yl)-3-fluoro-1-methylpiperidin-4-amine; |
| 603 | 4-((3-(3-(2,2-difluoroethyl)-7-(((3S,4S)-3-fluoro-1-methylpiperidin-4-yl)amino)benzofuran-2-yl)prop-2-yn- |
| | 1-yl)amino)-3-methoxy-N-methylbenzamide; |
| 604 | (3S,4S)-N-(3-(2,2-difluoroethyl)-2-(3-((2-methoxy-4-(methylsulfonyl)phenyl)amino)prop-1-yn-1-yl)benzofuran- |
| | 7-yl)-3-fluoro-1-methylpiperidin-4-amine; |
| 605 | 4-((3-(3-(2,2-difluoroethyl)-7-((4-(dimethylamino)cyclohexyl)amino)benzofuran-2-yl)prop-2-yn-1-yl)amino)- |
| | 3-methoxybenzenesulfonamide; |
| 606 | N1-(3-(2,2-difluoroethyl)-2-(3-((5-fluoro-2-methoxy-4-(methylsulfonyl)phenyl)amino)prop-1-yn-1-yl)benzo- |
| | furan-7-yl)-N4,N4-dimethylcyclohexane-1,4-diamine; |
| 607 | 4-((3-(3-(2,2-difluoroethyl)-7-((4-(dimethylamino)cyclohexyl)amino)benzofuran-2-yl)prop-2-yn-1-yl)amino)- |
| | 3-methoxy-N-methylbenzamide; |
| 608 | N1-(3-(2,2-difluoroethyl)-2-(3-((2-methoxy-4-(methylsulfonyl)phenyl)amino)prop-1-yn-1-yl)benzofuran-7- |
| | yl)-N4,N4-dimethylcyclohexane-1,4-diamine; |
| 609 | 4-((3-(3-(2,2-difluoroethyl)-7-((2-(dimethylamino)ethyl)amino)benzofuran-2-yl)prop-2-yn-1-yl)amino)-3- |
| | methoxybenzenesulfonamide; |
| 610 | N1-(3-(2,2-difluoroethyl)-2-(3-((5-fluoro-2-methoxy-4-(methylsulfonyl)phenyl)amino)prop-1-yn-1-yl)benzo- |
| | furan-7-yl)-N2,N2-dimethylethane-1,2-diamine; |
| 611 | 4-((3-(3-(2,2-difluoroethyl)-7-((2-(dimethylamino)ethyl)amino)benzofuran-2-yl)prop-2-yn-1-yl)amino)-3- |
| | methoxy-N-methylbenzamide; |
| 612 | N1-(3-(2,2-difluoroethyl)-2-(3-((2-methoxy-4-(methylsulfonyl)phenyl)amino)prop-1-yn-1-yl)benzofuran-7- |
| | yl)-N2,N2-dimethylethane-1,2-diamine; |
| 613 | 4-((3-(3-(2,2-difluoroethyl)-7-(((3S,4R)-3-fluoro-1-methylpiperidin-4-yl)amino)benzofuran-2-yl)prop-2-yn- |
| | 1-yl)amino)-3-methoxybenzenesulfonamide; |
| 614 | (3S,4R)-N-(3-(2,2-difluoroethyl)-2-(3-((5-fluoro-2-methoxy-4-(methylsulfonyl)phenyl)amino)prop-1-yn-1- |
| | yl)benzofuran-7-yl)-3-fluoro-1-methylpiperidin-4-amine; |
| 615 | 4-((3-(3-(2,2-difluoroethyl)-7-(((3S,4R)-3-fluoro-1-methylpiperidin-4-yl)amino)benzofuran-2-yl)prop-2-yn- |
| | 1-yl)amino)-3-methoxy-N-methylbenzamide; |
| 616 | (3S,4R)-N-(3-(2,2-difluoroethyl)-2-(3-((2-methoxy-4-(methylsulfonyl)phenyl)amino)prop-1-yn-1-yl)benzofuran- |
| | 7-yl)-3-fluoro-1-methylpiperidin-4-amine; |
| 617 | 4-((3-(3-(2,2-difluoroethyl)-7-((1-methylpiperidin-4-yl)amino)benzofuran-2-yl)prop-2-yn-1-yl)amino)-3- |
| | methoxybenzenesulfonamide; |
| 618 | N-(3-(2,2-difluoroethyl)-2-(3-((5-fluoro-2-methoxy-4-(methylsulfonyl)phenyl)amino)prop-1-yn-1-yl)benzofuran- |
| | 7-yl)-1-methylpiperidin-4-amine; |
| 619 | 4-((3-(3-(2,2-difluoroethyl)-7-((1-methylpiperidin-4-yl)amino)benzofuran-2-yl)prop-2-yn-1-yl)amino)-3- |
| | methoxy-N-methylbenzamide; |
| 620 | N-(3-(2,2-difluoroethyl)-2-(3-((2-methoxy-4-(methylsulfonyl)phenyl)amino)prop-1-yn-1-yl)benzofuran-7-yl)- |
| | 1-methylpiperidin-4-amine; |
| 621 | 4-((3-(3-(2,2-difluoroethyl)-7-((tetrahydro-2H-pyran-4-yl)amino)benzofuran-2-yl)prop-2-yn-1-yl)amino)-3- |
| | methoxybenzenesulfonamide; |
| 622 | 3-(2,2-difluoroethyl)-2-(3-((5-fluoro-2-methoxy-4-(methylsulfonyl)phenyl)amino)prop-1-yn-1-yl)-N-(tetrahydro- |
| | 2H-pyran-4-yl)benzofuran-7-amine; |
| 623 | 4-((3-(3-(2,2-difluoroethyl)-7-((tetrahydro-2H-pyran-4-yl)amino)benzofuran-2-yl)prop-2-yn-1-yl)amino)-3- |
| | methoxy-N-methylbenzamide; |
| 624 | 3-(2,2-difluoroethyl)-2-(3-((2-methoxy-4-(methylsulfonyl)phenyl)amino)prop-1-yn-1-yl)-N-(tetrahydro-2H- |
| | pyran-4-yl)benzofuran-7-amine; |
| 625 | 4-((3-(7-((3,3-difluoro-1-methylpiperidin-4-yl)amino)-3-(1H-pyrrol-1-yl)benzofuran-2-yl)prop-2-yn-1-yl)amino)- |
| | 3-methoxybenzenesulfonamide; |
| 626 | 3,3-difluoro-N-(2-(3-((5-fluoro-2-methoxy-4-(methylsulfonyl)phenyl)amino)prop-1-yn-1-yl)-3-(1H-pyrrol- |
| | 1-yl)benzofuran-7-yl)-1-methylpiperidin-4-amine; |
| 627 | 4-((3-(7-((3,3-difluoro-1-methylpiperidin-4-yl)amino)-3-(1H-pyrrol-1-yl)benzofuran-2-yl)prop-2-yn-1-yl)amino)- |
| | 3-methoxy-N-methylbenzamide; |
| 628 | 3,3-difluoro-N-(2-(3-((2-methoxy-4-(methylsulfonyl)phenyl)amino)prop-1-yn-1-yl)-3-(1H-pyrrol-1-yl)benzofuran- |
| | 7-yl)-1-methylpiperidin-4-amine; |
| 629 | 4-((3-(7-((1,1-dioxidotetrahydro-2H-thiopyran-4-yl)amino)-3-(1H-pyrrol-1-yl)benzofuran-2-yl)prop-2-yn-1- |
| | yl)amino)-3-methoxybenzenesulfonamide; |
| 630 | 4-((2-(3-((5-fluoro-2-methoxy-4-(methylsulfonyl)phenyl)amino)prop-1-yn-1-yl)-3-(1H-pyrrol-1-yl)benzofuran- |
| | 7-yl)amino)tetrahydro-2H-thiopyran 1,1-dioxide; |
| 631 | 4-((3-(7-((1,1-dioxidotetrahydro-2H-thiopyran-4-yl)amino)-3-(1H-pyrrol-1-yl)benzofuran-2-yl)prop-2-yn-1- |
| | yl)amino)-3-methoxy-N-methylbenzamide; |
| 632 | 4-((2-(3-((2-methoxy-4-(methylsulfonyl)phenyl)amino)prop-1-yn-1-yl)-3-(1H-pyrrol-1-yl)benzofuran-7-yl) |
| | amino)tetrahydro-2H-thiopyran 1,1-dioxide; |
| 633 | 4-((3-(7-(((3R,4S)-3-fluoro-1-methylpiperidin-4-yl)amino)-3-(1H-pyrrol-1-yl)benzofuran-2-yl)prop-2-yn-1- |
| | yl)amino)-3-methoxybenzenesulfonamide; |
| 634 | (3R,4S)-3-fluoro-N-(2-(3-((5-fluoro-2-methoxy-4-(methylsulfonyl)phenyl)amino)prop-1-yn-1-yl)-3-(1H-pyrrol- |
| | 1-yl)benzofuran-7-yl)-1-methylpiperidin-4-amine; |
| 635 | 4-((3-(7-(((3R,4S)-3-fluoro-1-methylpiperidin-4-yl)amino)-3-(1H-pyrrol-1-yl)benzofuran-2-yl)prop-2-yn-1- |
| | yl)amino)-3-methoxy-N-methylbenzamide; |
| 636 | (3R,4S)-3-fluoro-N-(2-(3-((2-methoxy-4-(methylsulfonyl)phenyl)amino)prop-1-yn-1-yl)-3-(1H-pyrrol-1-yl) |
| | benzofuran-7-yl)-1-methylpiperidin-4-amine; |
| 637 | 4-((3-(7-((3-fluoro-1-methylpiperidin-4-yl)amino)-3-(1H-pyrrol-1-yl)benzofuran-2-yl)prop-2-yn-1-yl)amino)- |
| | 3-methoxybenzenesulfonamide; |
| 638 | 3-fluoro-N-(2-(3-((5-fluoro-2-methoxy-4-(methylsulfonyl)phenyl)amino)prop-1-yn-1-yl)-3-(1H-pyrrol-1- |
| | yl)benzofuran-7-yl)-1-methylpiperidin-4-amine; |
| 639 | 4-((3-(7-((3-fluoro-1-methylpiperidin-4-yl)amino)-3-(1H-pyrrol-1-yl)benzofuran-2-yl)prop-2-yn-1-yl)amino)- |
| | 3-methoxy-N-methylbenzamide; |
| 640 | 3-fluoro-N-(2-(3-((2-methoxy-4-(methylsulfonyl)phenyl)amino)prop-1-yn-1-yl)-3-(1H-pyrrol-1-yl)benzofuran- |
| | 7-yl)-1-methylpiperidin-4-amine; |
| 641 | 4-((3-(7-((1-(dimethylglycyl)piperidin-4-yl)amino)-3-(1H-pyrrol-1-yl)benzofuran-2-yl)prop-2-yn-1-yl)amino)- |
| | 3-methoxybenzenesulfonamide; |
| 642 | 2-(dimethylamino)-1-(4-((2-(3-((5-fluoro-2-methoxy-4-(methylsulfonyl)phenyl)amino)prop-1-yn-1-yl)-3-(1H- |
| | pyrrol-1-yl)benzofuran-7-yl)amino)piperidin-1-yl)ethan-1-one; |
| 643 | 4-((3-(7-((1-(dimethylglycyl)piperidin-4-yl)amino)-3-(1H-pyrrol-1-yl)benzofuran-2-yl)prop-2-yn-1-yl)amino)- |
| | 3-methoxy-N-methylbenzamide; |
| 644 | 2-(dimethylamino)-1-(4-((2-(3-((2-methoxy-4-(methylsulfonyl)phenyl)amino)prop-1-yn-1-yl)-3-(1H-pyrrol- |
| | 1-yl)benzofuran-7-yl)amino)piperidin-1-yl)ethan-1-one; |
| 645 | 4-((3-(7-((3-fluoro-1-(2-hydroxy-3-methoxypropyl)piperidin-4-yl)amino)-3-(1H-pyrrol-1-yl)benzofuran-2- |
| | yl)prop-2-yn-1-yl)amino)-3-methoxybenzenesulfonamide; |
| 646 | 1-(3-fluoro-4-((2-(3-((5-fluoro-2-methoxy-4-(methylsulfonyl)phenyl)amino)prop-1-yn-1-yl)-3-(1H-pyrrol-1- |
| | yl)benzofuran-7-yl)amino)piperidin-1-yl)-3-methoxypropan-2-ol; |
| 647 | 4-((3-(7-((3-fluoro-1-(2-hydroxy-3-methoxypropyl)piperidin-4-yl)amino)-3-(1H-pyrrol-1-yl)benzofuran-2- |
| | yl)prop-2-yn-1-yl)amino)-3-methoxy-N-methylbenzamide; |
| 648 | 1-(3-fluoro-4-((2-(3-((2-methoxy-4-(methylsulfonyl)phenyl)amino)prop-1-yn-1-yl)-3-(1H-pyrrol-1-yl)benzofuran- |
| | 7-yl)amino)piperidin-1-yl)-3-methoxypropan-2-ol; |
| 649 | 4-((3-(7-(((3R,4R)-3-fluoro-1-methylpiperidin-4-yl)amino)-3-(1H-pyrrol-1-yl)benzofuran-2-yl)prop-2-yn-1- |
| | yl)amino)-3-methoxybenzenesulfonamide; |
| 650 | (3R,4R)-3-fluoro-N-(2-(3-((5-fluoro-2-methoxy-4-(methylsulfonyl)phenyl)amino)prop-1-yn-1-yl)-3-(1H-pyrrol- |
| | 1-yl)benzofuran-7-yl)-1-methylpiperidin-4-amine; |
| 651 | 4-((3-(7-(((3R,4R)-3-fluoro-1-methylpiperidin-4-yl)amino)-3-(1H-pyrrol-1-yl)benzofuran-2-yl)prop-2-yn-1- |
| | yl)amino)-3-methoxy-N-methylbenzamide; |
| 652 | (3R,4R)-3-fluoro-N-(2-(3-((2-methoxy-4-(methylsulfonyl)phenyl)amino)prop-1-yn-1-yl)-3-(1H-pyrrol-1- |
| | yl)benzofuran-7-yl)-1-methylpiperidin-4-amine; |
| 653 | 4-((3-(7-(((3S,4S)-3-fluoro-1-methylpiperidin-4-yl)amino)-3-(1H-pyrrol-1-yl)benzofuran-2-yl)prop-2-yn-1- |
| | yl)amino)-3-methoxybenzenesulfonamide; |
| 654 | (3S,4S)-3-fluoro-N-(2-(3-((5-fluoro-2-methoxy-4-(methylsulfonyl)phenyl)amino)prop-1-yn-1-yl)-3-(1H-pyrrol- |
| | 1-yl)benzofuran-7-yl)-1-methylpiperidin-4-amine; |
| 655 | 4-((3-(7-(((3S,4S)-3-fluoro-1-methylpiperidin-4-yl)amino)-3-(1H-pyrrol-1-yl)benzofuran-2-yl)prop-2-yn-1- |
| | yl)amino)-3-methoxy-N-methylbenzamide; |
| 656 | (3S,4S)-3-fluoro-N-(2-(3-((2-methoxy-4-(methylsulfonyl)phenyl)amino)prop-1-yn-1-yl)-3-(1H-pyrrol-1-yl) |
| | benzofuran-7-yl)-1-methylpiperidin-4-amine; |
| 657 | 4-((3-(7-((4-(dimethylamino)cyclohexyl)amino)-3-(1H-pyrrol-1-yl)benzofuran-2-yl)prop-2-yn-1-yl)amino)- |
| | 3-methoxybenzenesulfonamide; |
| 658 | N1-(2-(3-((5-fluoro-2-methoxy-4-(methylsulfonyl)phenyl)amino)prop-1-yn-1-yl)-3-(1H-pyrrol-1-yl)benzofuran- |
| | 7-yl)-N4,N4-dimethylcyclohexane-1,4-diamine; |
| 659 | 4-((3-(7-((4-(dimethylamino)cyclohexyl)amino)-3-(1H-pyrrol-1-yl)benzofuran-2-yl)prop-2-yn-1-yl)amino)- |
| | 3-methoxy-N-methylbenzamide; |
| 660 | N1-(2-(3-((2-methoxy-4-(methylsulfonyl)phenyl)amino)prop-1-yn-1-yl)-3-(1H-pyrrol-1-yl)benzofuran-7-yl)- |
| | N4,N4-dimethylcyclohexane-1,4-diamine; |
| 661 | 4-((3-(7-((2-(dimethylamino)ethyl)amino)-3-(1H-pyrrol-1-yl)benzofuran-2-yl)prop-2-yn-1-yl)amino)-3- |
| | methoxybenzenesulfonamide; |
| 662 | N1-(2-(3-((5-fluoro-2-methoxy-4-(methylsulfonyl)phenyl)amino)prop-1-yn-1-yl)-3-(1H-pyrrol-1-yl)benzofuran- |
| | 7-yl)-N2,N2-dimethylethane-1,2-diamine; |
| 663 | 4-((3-(7-((2-(dimethylamino)ethyl)amino)-3-(1H-pyrrol-1-yl)benzofuran-2-yl)prop-2-yn-1-yl)amino)-3-methoxy- |
| | N-methylbenzamide; |
| 664 | N1-(2-(3-((2-methoxy-4-(methylsulfonyl)phenyl)amino)prop-1-yn-1-yl)-3-(1H-pyrrol-1-yl)benzofuran-7-yl)- |
| | N2,N2-dimethylethane-1,2-diamine; |
| 665 | 4-((3-(7-(((3S,4R)-3-fluoro-1-methylpiperidin-4-yl)amino)-3-(1H-pyrrol-1-yl)benzofuran-2-yl)prop-2-yn-1- |
| | yl)amino)-3-methoxybenzenesulfonamide; |
| 666 | (3S,4R)-3-fluoro-N-(2-(3-((5-fluoro-2-methoxy-4-(methylsulfonyl)phenyl)amino)prop-1-yn-1-yl)-3-(1H-pyrrol- |
| | 1-yl)benzofuran-7-yl)-1-methylpiperidin-4-amine; |
| 667 | 4-((3-(7-(((3S,4R)-3-fluoro-1-methylpiperidin-4-yl)amino)-3-(1H-pyrrol-1-yl)benzofuran-2-yl)prop-2-yn-1- |
| | yl)amino)-3-methoxy-N-methylbenzamide; |
| 668 | (3S,4R)-3-fluoro-N-(2-(3-((2-methoxy-4-(methylsulfonyl)phenyl)amino)prop-1-yn-1-yl)-3-(1H-pyrrol-1-yl) |
| | benzofuran-7-yl)-1-methylpiperidin-4-amine; |
| 669 | 3-methoxy-4-((3-(7-((1-methylpiperidin-4-yl)amino)-3-(1H-pyrrol-1-yl)benzofuran-2-yl)prop-2-yn-1-yl)amino) |
| | benzenesulfonamide; |
| 670 | N-(2-(3-((5-fluoro-2-methoxy-4-(methylsulfonyl)phenyl)amino)prop-1-yn-1-yl)-3-(1H-pyrrol-1-yl)benzofuran- |
| | 7-yl)-1-methylpiperidin-4-amine; |
| 671 | 3-methoxy-N-methyl-4-((3-(7-((1-methylpiperidin-4-yl)amino)-3-(1H-pyrrol-1-yl)benzofuran-2-yl)prop-2-yn- |
| | 1-yl)amino)benzamide; |
| 672 | N-(2-(3-((2-methoxy-4-(methylsulfonyl)phenyl)amino)prop-1-yn-1-yl)-3-(1H-pyrrol-1-yl)benzofuran-7-yl)- |
| | 1-methylpiperidin-4-amine; |
| 673 | 4-((3-(3-(1H-pyrrol-1-yl)-7-((tetrahydro-2H-pyran-4-yl)amino)benzofuran-2-yl)prop-2-yn-1-yl)amino)-3- |
| | methoxybenzenesulfonamide; |
| 674 | 2-(3-((5-fluoro-2-methoxy-4-(methylsulfonyl)phenyl)amino)prop-1-yn-1-yl)-3-(1H-pyrrol-1-yl)-N-(tetrahydro- |
| | 2H-pyran-4-yl)benzofuran-7-amine; |
| 675 | 4-((3-(3-(1H-pyrrol-1-yl)-7-((tetrahydro-2H-pyran-4-yl)amino)benzofuran-2-yl)prop-2-yn-1-yl)amino)-3- |
| | methoxy-N-methylbenzamide; |
| 676 | 2-(3-((2-methoxy-4-(methylsulfonyl)phenyl)amino)prop-1-yn-1-yl)-3-(1H-pyrrol-1-yl)-N-(tetrahydro-2H-pyran- |
| | 4-yl)benzofuran-7-amine; |
| 677 | 4-((3-(7-((3,3-difluoro-1-methylpiperidin-4-yl)amino)-3-ethylbenzofuran-2-yl)prop-2-yn-1-yl)amino)-3- |
| | methoxybenzenesulfonamide; |
| 678 | N-(3-ethyl-2-(3-((5-fluoro-2-methoxy-4-(methylsulfonyl)phenyl)amino)prop-1-yn-1-yl)benzofuran-7-yl)- |
| | 3,3-difluoro-1-methylpiperidin-4-amine; |
| 679 | 4-((3-(7-((3,3-difluoro-1-methylpiperidin-4-yl)amino)-3-ethylbenzofuran-2-yl)prop-2-yn-1-yl)amino)-3- |
| | methoxy-N-methylbenzamide; |
| 680 | N-(3-ethyl-2-(3-((2-methoxy-4-(methylsulfonyl)phenyl)amino)prop-1-yn-1-yl)benzofuran-7-yl)-3,3-difluoro- |
| | 1-methylpiperidin-4-amine; |
| 681 | 4-((3-(7-((1,1-dioxidotetrahydro-2H-thiopyran-4-yl)amino)-3-ethylbenzofuran-2-yl)prop-2-yn-1-yl)amino)- |
| | 3-methoxybenzenesulfonamide; |
| 682 | 4-((3-ethyl-2-(3-((5-fluoro-2-methoxy-4-(methylsulfonyl)phenyl)amino)prop-1-yn-1-yl)benzofuran-7-yl)amino) |
| | tetrahydro-2H-thiopyran 1,1-dioxide; |
| 683 | 4-((3-(7-((1,1-dioxidotetrahydro-2H-thiopyran-4-yl)amino)-3-ethylbenzofuran-2-yl)prop-2-yn-1-yl)amino)- |
| | 3-methoxy-N-methylbenzamide; |
| 684 | 4-((3-ethyl-2-(3-((2-methoxy-4-(methylsulfonyl)phenyl)amino)prop-1-yn-1-yl)benzofuran-7-yl)amino)tetra- |
| | hydro-2H-thiopyran 1,1-dioxide; |
| 685 | 4-((3-(3-ethyl-7-(((3R,4S)-3-fluoro-1-methylpiperidin-4-yl)amino)benzofuran-2-yl)prop-2-yn-1-yl)amino)- |
| | 3-methoxybenzenesulfonamide; |
| 686 | (3R,4S)-N-(3-ethyl-2-(3-((5-fluoro-2-methoxy-4-(methylsulfonyl)phenyl)amino)prop-1-yn-1-yl)benzofuran- |
| | 7-yl)-3-fluoro-1-methylpiperidin-4-amine; |
| 687 | 4-((3-(3-ethyl-7-(((3R,4S)-3-fluoro-1-methylpiperidin-4-yl)amino)benzofuran-2-yl)prop-2-yn-1-yl)amino)- |
| | 3-methoxy-N-methylbenzamide; |
| 688 | (3R,4S)-N-(3-ethyl-2-(3-((2-methoxy-4-(methylsulfonyl)phenyl)amino)prop-1-yn-1-yl)benzofuran-7-yl)-3-fluoro- |
| | 1-methylpiperidin-4-amine; |
| 689 | 4-((3-(3-ethyl-7-((3-fluoro-1-methylpiperidin-4-yl)amino)benzofuran-2-yl)prop-2-yn-1-yl)amino)-3- |
| | methoxybenzenesulfonamide; |
| 690 | N-(3-ethyl-2-(3-((5-fluoro-2-methoxy-4-(methylsulfonyl)phenyl)amino)prop-1-yn-1-yl)benzofuran-7-yl)-3- |
| | fluoro-1-methylpiperidin-4-amine; |
| 691 | 4-((3-(3-ethyl-7-((3-fluoro-1-methylpiperidin-4-yl)amino)benzofuran-2-yl)prop-2-yn-1-yl)amino)-3-methoxy- |
| | N-methylbenzamide; |
| 692 | N-(3-ethyl-2-(3-((2-methoxy-4-(methylsulfonyl)phenyl)amino)prop-1-yn-1-yl)benzofuran-7-yl)-3-fluoro-1- |
| | methylpiperidin-4-amine; |
| 693 | 4-((3-(7-((1-(dimethylglycyl)piperidin-4-yl)amino)-3-ethylbenzofuran-2-yl)prop-2-yn-1-yl)amino)-3- |
| | methoxybenzenesulfonamide; |
| 694 | 2-(dimethylamino)-1-(4-((3-ethyl-2-(3-((5-fluoro-2-methoxy-4-(methylsulfonyl)phenyl)amino)prop-1-yn-1- |
| | yl)benzofuran-7-yl)amino)piperidin-1-yl)ethan-1-one; |
| 695 | 4-((3-(7-((1-(dimethylglycyl)piperidin-4-yl)amino)-3-ethylbenzofuran-2-yl)prop-2-yn-1-yl)amino)-3-methoxy- |
| | N-methylbenzamide; |
| 696 | 2-(dimethylamino)-1-(4-((3-ethyl-2-(3-((2-methoxy-4-(methylsulfonyl)phenyl)amino)prop-1-yn-1-yl)benzo- |
| | furan-7-yl)amino)piperidin-1-yl)ethan-1-one; |
| 697 | 4-((3-(3-ethyl-7-((3-fluoro-1-(2-hydroxy-3-methoxypropyl)piperidin-4-yl)amino)benzofuran-2-yl)prop-2-yn- |
| | 1-yl)amino)-3-methoxybenzenesulfonamide; |
| 698 | 1-(4-((3-ethyl-2-(3-((5-fluoro-2-methoxy-4-(methylsulfonyl)phenyl)amino)prop-1-yn-1-yl)benzofuran-7-yl) |
| | amino)-3-fluoropiperidin-1-yl)-3-methoxypropan-2-ol; |
| 699 | 4-((3-(3-ethyl-7-((3-fluoro-1-(2-hydroxy-3-methoxypropyl)piperidin-4-yl)amino)benzofuran-2-yl)prop-2-yn- |
| | 1-yl)amino)-3-methoxy-N-methylbenzamide; |
| 700 | 1-(4-((3-ethyl-2-(3-((2-methoxy-4-(methylsulfonyl)phenyl)amino)prop-1-yn-1-yl)benzofuran-7-yl)amino)-3- |
| | fluoropiperidin-1-yl)-3-methoxypropan-2-ol; |
| 701 | 4-((3-(3-ethyl-7-(((3R,4R)-3-fluoro-1-methylpiperidin-4-yl)amino)benzofuran-2-yl)prop-2-yn-1-yl)amino)- |
| | 3-methoxybenzenesulfonamide; |
| 702 | (3R,4R)-N-(3-ethyl-2-(3-((5-fluoro-2-methoxy-4-(methylsulfonyl)phenyl)amino)prop-1-yn-1-yl)benzofuran- |
| | 7-yl)-3-fluoro-1-methylpiperidin-4-amine; |
| 703 | 4-((3-(3-ethyl-7-(((3R,4R)-3-fluoro-1-methylpiperidin-4-yl)amino)benzofuran-2-yl)prop-2-yn-1-yl)amino)- |
| | 3-methoxy-N-methylbenzamide; |
| 704 | (3R,4R)-N-(3-ethyl-2-(3-((2-methoxy-4-(methylsulfonyl)phenyl)amino)prop-1-yn-1-yl)benzofuran-7-yl)-3- |
| | fluoro-1-methylpiperidin-4-amine; |
| 705 | 4-((3-(3-ethyl-7-(((3S,4S)-3-fluoro-1-methylpiperidin-4-yl)amino)benzofuran-2-yl)prop-2-yn-1-yl)amino)-3- |
| | methoxybenzenesulfonamide; |
| 706 | (3S,4S)-N-(3-ethyl-2-(3-((5-fluoro-2-methoxy-4-(methylsulfonyl)phenyl)amino)prop-1-yn-1-yl)benzofuran- |
| | 7-yl)-3-fluoro-1-methylpiperidin-4-amine; |
| 707 | 4-((3-(3-ethyl-7-(((3S,4S)-3-fluoro-1-methylpiperidin-4-yl)amino)benzofuran-2-yl)prop-2-yn-1-yl)amino)-3- |
| | methoxy-N-methylbenzamide; |
| 708 | (3S,4S)-N-(3-ethyl-2-(3-((2-methoxy-4-(methylsulfonyl)phenyl)amino)prop-1-yn-1-yl)benzofuran-7-yl)-3- |
| | fluoro-1-methylpiperidin-4-amine; |
| 709 | 4-((3-(7-((4-(dimethylamino)cyclohexyl)amino)-3-ethylbenzofuran-2-yl)prop-2-yn-1-yl)amino)-3-methoxy- |
| | benzenesulfonamide; |
| 710 | N1-(3-ethyl-2-(3-((5-fluoro-2-methoxy-4-(methylsulfonyl)phenyl)amino)prop-1-yn-1-yl)benzofuran-7-yl)- |
| | N4,N4-dimethylcyclohexane-1,4-diamine; |
| 711 | 4-((3-(7-((4-(dimethylamino)cyclohexyl)amino)-3-ethylbenzofuran-2-yl)prop-2-yn-1-yl)amino)-3-methoxy- |
| | N-methylbenzamide; |
| 712 | N1-(3-ethyl-2-(3-((2-methoxy-4-(methylsulfonyl)phenyl)amino)prop-1-yn-1-yl)benzofuran-7-yl)-N4,N4- |
| | dimethylcyclohexane-1,4-diamine; |
| 713 | 4-((3-(7-((2-(dimethylamino)ethyl)amino)-3-ethylbenzofuran-2-yl)prop-2-yn-1-yl)amino)-3- |
| | methoxybenzenesulfonamide; |
| 714 | N1-(3-ethyl-2-(3-((5-fluoro-2-methoxy-4-(methylsulfonyl)phenyl)amino)prop-1-yn-1-yl)benzofuran-7-yl)- |
| | N2,N2-dimethylethane-1,2-diamine; |
| 715 | 4-((3-(7-((2-(dimethylamino)ethyl)amino)-3-ethylbenzofuran-2-yl)prop-2-yn-1-yl)amino)-3-methoxy-N- |
| | methylbenzamide; |
| 716 | N1-(3-ethyl-2-(3-((2-methoxy-4-(methylsulfonyl)phenyl)amino)prop-1-yn-1-yl)benzofuran-7-yl)-N2,N2- |
| | dimethylethane-1,2-diamine; |
| 717 | 4-((3-(3-ethyl-7-(((3S,4R)-3-fluoro-1-methylpiperidin-4-yl)amino)benzofuran-2-yl)prop-2-yn-1-yl)amino)- |
| | 3-methoxybenzenesulfonamide; |
| 718 | (3S,4R)-N-(3-ethyl-2-(3-((5-fluoro-2-methoxy-4-(methylsulfonyl)phenyl)amino)prop-1-yn-1-yl)benzofuran- |
| | 7-yl)-3-fluoro-1-methylpiperidin-4-amine; |
| 719 | 4-((3-(3-ethyl-7-(((3S,4R)-3-fluoro-1-methylpiperidin-4-yl)amino)benzofuran-2-yl)prop-2-yn-1-yl)amino)- |
| | 3-methoxy-N-methylbenzamide; |
| 720 | (3S,4R)-N-(3-ethyl-2-(3-((2-methoxy-4-(methylsulfonyl)phenyl)amino)prop-1-yn-1-yl)benzofuran-7-yl)-3- |
| | fluoro-1-methylpiperidin-4-amine; |
| 721 | 4-((3-(3-ethyl-7-((1-methylpiperidin-4-yl)amino)benzofuran-2-yl)prop-2-yn-1-yl)amino)-3- |
| | methoxybenzenesulfonamide; |
| 722 | N-(3-ethyl-2-(3-((5-fluoro-2-methoxy-4-(methylsulfonyl)phenyl)amino)prop-1-yn-1-yl)benzofuran-7-yl)-1- |
| | methylpiperidin-4-amine; |
| 723 | 4-((3-(3-ethyl-7-((1-methylpiperidin-4-yl)amino)benzofuran-2-yl)prop-2-yn-1-yl)amino)-3-methoxy-N- |
| | methylbenzamide; |
| 724 | N-(3-ethyl-2-(3-((2-methoxy-4-(methylsulfonyl)phenyl)amino)prop-1-yn-1-yl)benzofuran-7-yl)-1- |
| | methylpiperidin-4-amine; |
| 725 | 4-((3-(3-ethyl-7-((tetrahydro-2H-pyran-4-yl)amino)benzofuran-2-yl)prop-2-yn-1-yl)amino)-3- |
| | methoxybenzenesulfonamide; |
| 726 | 3-ethyl-2-(3-((5-fluoro-2-methoxy-4-(methylsulfonyl)phenyl)amino)prop-1-yn-1-yl)-N-(tetrahydro-2H- |
| | pyran-4-yl)benzofuran-7-amine; |
| 727 | 4-((3-(3-ethyl-7-((tetrahydro-2H-pyran-4-yl)amino)benzofuran-2-yl)prop-2-yn-1-yl)amino)-3-methoxy-N- |
| | methylbenzamide; |
| 728 | 3-ethyl-2-(3-((2-methoxy-4-(methylsulfonyl)phenyl)amino)prop-1-yn-1-yl)-N-(tetrahydro-2H-pyran-4- |
| | yl)benzofuran-7-amine; |
| 729 | 3-(cyclopropylmethyl)-2-(3-((2-methoxy-4-(methylsulfonyl)phenyl)amino)prop-1-yn-1-yl)-N-(1- |
| | methylpiperidin-4-yl)thieno[3,2-b]pyridin-7-amine; |
| 730 | 2-(3-((2-methoxy-4-(methylsulfonyl)phenyl)amino)prop-1-yn-1-yl)-N-(1-methylpiperidin-4-yl)-3-(2,2,2- |
| | trifluoroethyl)thieno[3,2-b]pyridin-7-amine; |
| 731 | 3-ethyl-2-(3-((2-methoxy-4-(methylsulfonyl)phenyl)amino)prop-1-yn-1-yl)-N-(1-methylpiperidin-4- |
| | yl)thieno[3,2-b]pyridin-7-amine; |
| 732 | 4-((3-(3-(cyclopropylmethyl)-7-((1-methylpiperidin-4-yl)amino)thieno[3,2-b]pyridin-2-yl)prop-2-yn-1- |
| | yl)amino)-3-methoxy-N-methylbenzamide; |
| 733 | 3-methoxy-N-methyl-4-((3-(7-((1-methylpiperidin-4-yl)amino)-3-(2,2,2-trifluoroethyl)thieno[3,2-b]pyridin- |
| | 2-yl)prop-2-yn-1-yl)amino)benzamide; |
| 734 | 4-((3-(3-ethyl-7-((1-methylpiperidin-4-yl)amino)thieno[3,2-b]pyridin-2-yl)prop-2-yn-1-yl)amino)-3-methoxy- |
| | N-methylbenzamide; |
| 735 | 4-((3-(3-(cyclopropylmethyl)-7-((1-methylpiperidin-4-yl)amino)thieno[3,2-b]pyridin-2-yl)prop-2-yn-1- |
| | yl)amino)-3-methoxybenzenesulfonamide; |
| 736 | 3-methoxy-4-((3-(7-((1-methylpiperidin-4-yl)amino)-3-(2,2,2-trifluoroethyl)thieno[3,2-b]pyridin-2-yl)prop- |
| | 2-yn-1-yl)amino)benzenesulfonamide; |
| 737 | 4-((3-(3-ethyl-7-((1-methylpiperidin-4-yl)amino)thieno[3,2-b]pyridin-2-yl)prop-2-yn-1-yl)amino)-3- |
| | methoxybenzenesulfonamide; |
| 738 | 1-(4-((3-(cyclopropylmethyl)-2-(3-((2-methoxy-4-(methylsulfonyl)phenyl)amino)prop-1-yn-1-yl)thieno[3,2- |
| | b]pyridin-7-yl)amino)-3-fluoropiperidin-1-yl)-3-methoxypropan-2-ol; |
| 739 | 1-(3-fluoro-4-((2-(3-((2-methoxy-4-(methylsulfonyl)phenyl)amino)prop-1-yn-1-yl)-3-(2,2,2-trifluoroethyl) |
| | thieno[3,2-b]pyridin-7-yl)amino)piperidin-1-yl)-3-methoxypropan-2-ol; |
| 740 | 1-(4-((3-ethyl-2-(3-((2-methoxy-4-(methylsulfonyl)phenyl)amino)prop-1-yn-1-yl)thieno[3,2-b]pyridin-7-yl) |
| | amino)-3-fluoropiperidin-1-yl)-3-methoxypropan-2-ol; |
| 741 | 4-((3-(3-(cyclopropylmethyl)-7-((3-fluoro-1-(2-hydroxy-3-methoxypropyl)piperidin-4-yl)amino)thieno[3,2- |
| | b]pyridin-2-yl)prop-2-yn-1-yl)amino)-3-methoxy-N-methylbenzamide; |
| 742 | 4-((3-(7-((3-fluoro-1-(2-hydroxy-3-methoxypropyl)piperidin-4-yl)amino)-3-(2,2,2-trifluoroethyl)thieno[3,2- |
| | b]pyridin-2-yl)prop-2-yn-1-yl)amino)-3-methoxy-N-methylbenzamide; |
| 743 | 4-((3-(3-ethyl-7-((3-fluoro-1-(2-hydroxy-3-methoxypropyl)piperidin-4-yl)amino)thieno[3,2-b]pyridin-2-yl) |
| | prop-2-yn-1-yl)amino)-3-methoxy-N-methylbenzamide; |
| 744 | 4-((3-(3-(cyclopropylmethyl)-7-((3-fluoro-1-(2-hydroxy-3-methoxypropyl)piperidin-4-yl)amino)thieno[3,2- |
| | b]pyridin-2-yl)prop-2-yn-1-yl)amino)-3-methoxybenzenesulfonamide; |
| 745 | 4-((3-(7-((3-fluoro-1-(2-hydroxy-3-methoxypropyl)piperidin-4-yl)amino)-3-(2,2,2-trifluoroethyl)thieno[3,2- |
| | b]pyridin-2-yl)prop-2-yn-1-yl)amino)-3-methoxybenzenesulfonamide; |
| 746 | 4-((3-(3-ethyl-7-((3-fluoro-1-(2-hydroxy-3-methoxypropyl)piperidin-4-yl)amino)thieno[3,2-b]pyridin-2-yl) |
| | prop-2-yn-1-yl)amino)-3-methoxybenzenesulfonamide; |
| 747 | 3-(cyclopropylmethyl)-N-(3-fluoro-1-methylpiperidin-4-yl)-2-(3-((2-methoxy-4-(methylsulfonyl)phenyl)amino) |
| | prop-1-yn-1-yl)thieno[3,2-b]pyridin-7-amine; |
| 748 | N-(3-fluoro-1-methylpiperidin-4-yl)-2-(3-((2-methoxy-4-(methylsulfonyl)phenyl)amino)prop-1-yn-1-yl)-3- |
| | (2,2,2-trifluoroethyl)thieno[3,2-b]pyridin-7-amine; |
| 749 | 3-ethyl-N-(3-fluoro-1-methylpiperidin-4-yl)-2-(3-((2-methoxy-4-(methylsulfonyl)phenyl)amino)prop-1-yn- |
| | 1-yl)thieno[3,2-b]pyridin-7-amine; |
| 750 | 4-((3-(3-(cyclopropylmethyl)-7-((3-fluoro-1-methylpiperidin-4-yl)amino)thieno[3,2-b]pyridin-2-yl)prop-2- |
| | yn-1-yl)amino)-3-methoxy-N-methylbenzamide; |
| 751 | 4-((3-(7-((3-fluoro-1-methylpiperidin-4-yl)amino)-3-(2,2,2-trifluoroethyl)thieno[3,2-b]pyridin-2-yl)prop-2- |
| | yn-1-yl)amino)-3-methoxy-N-methylbenzamide; |
| 752 | 4-((3-(3-ethyl-7-((3-fluoro-1-methylpiperidin-4-yl)amino)thieno[3,2-b]pyridin-2-yl)prop-2-yn-1-yl)amino)- |
| | 3-methoxy-N-methylbenzamide; |
| 753 | 4-((3-(3-(cyclopropylmethyl)-7-((3-fluoro-1-methylpiperidin-4-yl)amino)thieno[3,2-b]pyridin-2-yl)prop-2- |
| | yn-1-yl)amino)-3-methoxybenzenesulfonamide; |
| 754 | 4-((3-(7-((3-fluoro-1-methylpiperidin-4-yl)amino)-3-(2,2,2-trifluoroethyl)thieno[3,2-b]pyridin-2-yl)prop-2- |
| | yn-1-yl)amino)-3-methoxybenzenesulfonamide; |
| 755 | 4-((3-(3-ethyl-7-((3-fluoro-1-methylpiperidin-4-yl)amino)thieno[3,2-b]pyridin-2-yl)prop-2-yn-1-yl)amino)- |
| | 3-methoxybenzenesulfonamide; |
| 756 | N1-(3-(cyclopropylmethyl)-2-(3-((2-methoxy-4-(methylsulfonyl)phenyl)amino)prop-1-yn-1-yl)thieno[3,2- |
| | b]pyridin-7-yl)-N4,N4-dimethylcyclohexane-1,4-diamine; |
| 757 | N1-(2-(3-((2-methoxy-4-(methylsulfonyl)phenyl)amino)prop-1-yn-1-yl)-3-(2,2,2-trifluoroethyl)thieno[3,2- |
| | b]pyridin-7-yl)-N4,N4-dimethylcyclohexane-1,4-diamine; |
| 758 | N1-(3-ethyl-2-(3-((2-methoxy-4-(methylsulfonyl)phenyl)amino)prop-1-yn-1-yl)thieno[3,2-b]pyridin-7-yl)- |
| | N4,N4-dimethylcyclohexane-1,4-diamine; |
| 759 | 4-((3-(3-(cyclopropylmethyl)-7-((4-(dimethylamino)cyclohexyl)amino)thieno[3,2-b]pyridin-2-yl)prop-2-yn- |
| | 1-yl)amino)-3-methoxy-N-methylbenzamide; |
| 760 | 4-((3-(7-((4-(dimethylamino)cyclohexyl)amino)-3-(2,2,2-trifluoroethyl)thieno[3,2-b]pyridin-2-yl)prop-2-yn- |
| | 1-yl)amino)-3-methoxy-N-methylbenzamide; |
| 761 | 4-((3-(7-((4-(dimethylamino)cyclohexyl)amino)-3-ethylthieno[3,2-b]pyridin-2-yl)prop-2-yn-1-yl)amino)-3- |
| | methoxy-N-methylbenzamide; |
| 762 | 4-((3-(3-(cyclopropylmethyl)-7-((4-(dimethylamino)cyclohexyl)amino)thieno[3,2-b]pyridin-2-yl)prop-2-yn- |
| | 1-yl)amino)-3-methoxybenzenesulfonamide; |
| 763 | 4-((3-(7-((4-(dimethylamino)cyclohexyl)amino)-3-(2,2,2-trifluoroethyl)thieno[3,2-b]pyridin-2-yl)prop-2-yn- |
| | 1-yl)amino)-3-methoxybenzenesulfonamide; |
| 764 | 4-((3-(7-((4-(dimethylamino)cyclohexyl)amino)-3-ethylthieno[3,2-b]pyridin-2-yl)prop-2-yn-1-yl)amino)-3- |
| | methoxybenzenesulfonamide; |
| 765 | 3-(cyclopropylmethyl)-N-(3,3-difluoro-1-methylpiperidin-4-yl)-2-(3-((2-methoxy-4-(methylsulfonyl)phenyl) |
| | amino)prop-1-yn-1-yl)thieno[3,2-b]pyridin-7-amine; |
| 766 | N-(3,3-difluoro-1-methylpiperidin-4-yl)-2-(3-((2-methoxy-4-(methylsulfonyl)phenyl)amino)prop-1-yn-1-yl)- |
| | 3-(2,2,2-trifluoroethyl)thieno[3,2-b]pyridin-7-amine; |
| 767 | N-(3,3-difluoro-1-methylpiperidin-4-yl)-3-ethyl-2-(3-((2-methoxy-4-(methylsulfonyl)phenyl)amino)prop-1- |
| | yn-1-yl)thieno[3,2-b]pyridin-7-amine; |
| 768 | 4-((3-(3-(cyclopropylmethyl)-7-((3,3-difluoro-1-methylpiperidin-4-yl)amino)thieno[3,2-b]pyridin-2-yl)prop- |
| | 2-yn-1-yl)amino)-3-methoxy-N-methylbenzamide; |
| 769 | 4-((3-(7-((3,3-difluoro-1-methylpiperidin-4-yl)amino)-3-(2,2,2-trifluoroethyl)thieno[3,2-b]pyridin-2-yl)prop- |
| | 2-yn-1-yl)amino)-3-methoxy-N-methylbenzamide; |
| 770 | 4-((3-(7-((3,3-difluoro-1-methylpiperidin-4-yl)amino)-3-ethylthieno[3,2-b]pyridin-2-yl)prop-2-yn-1-yl)amino)- |
| | 3-methoxy-N-methylbenzamide; |
| 771 | 4-((3-(3-(cyclopropylmethyl)-7-((3,3-difluoro-1-methylpiperidin-4-yl)amino)thieno[3,2-b]pyridin-2-yl)prop- |
| | 2-yn-1-yl)amino)-3-methoxybenzenesulfonamide; |
| 772 | 4-((3-(7-((3,3-difluoro-1-methylpiperidin-4-yl)amino)-3-(2,2,2-trifluoroethyl)thieno[3,2-b]pyridin-2-yl)prop- |
| | 2-yn-1-yl)amino)-3-methoxybenzenesulfonamide; |
| 773 | 4-((3-(7-((3,3-difluoro-1-methylpiperidin-4-yl)amino)-3-ethylthieno[3,2-b]pyridin-2-yl)prop-2-yn-1-yl) |
| | amino)-3-methoxybenzenesulfonamide; |
| 774 | 3-(cyclopropylmethyl)-2-(3-((2-methoxy-4-(methylsulfonyl)phenyl)amino)prop-1-yn-1-yl)-N-(1-methylpiperidin- |
| | 4-yl)thieno[3,2-c]pyridin-7-amine; |
| 775 | 4-((3-(3-(cyclopropylmethyl)-7-((1-methylpiperidin-4-yl)amino)thieno[3,2-c]pyridin-2-yl)prop-2-yn-1-yl)amino)- |
| | 3-methoxy-N-methylbenzamide; |
| 776 | 4-((3-(3-(cyclopropylmethyl)-7-((1-methylpiperidin-4-yl)amino)thieno[3,2-c]pyridin-2-yl)prop-2-yn-1-yl)amino)- |
| | 3-methoxybenzenesulfonamide; |
| 777 | 2-(3-((2-methoxy-4-(methylsulfonyl)phenyl)amino)prop-1-yn-1-yl)-N-(1-methylpiperidin-4-yl)-3-(2,2,2- |
| | trifluoroethyl)thieno[3,2-c]pyridin-7-amine; |
| 778 | 3-methoxy-N-methyl-4-((3-(7-((1-methylpiperidin-4-yl)amino)-3-(2,2,2-trifluoroethyl)thieno[3,2-c]pyridin- |
| | 2-yl)prop-2-yn-1-yl)amino)benzamide; |
| 779 | 3-methoxy-4-((3-(7-((1-methylpiperidin-4-yl)amino)-3-(2,2,2-trifluoroethyl)thieno[3,2-c]pyridin-2-yl)prop- |
| | 2-yn-1-yl)amino)benzenesulfonamide; |
| 780 | 3-ethyl-2-(3-((2-methoxy-4-(methylsulfonyl)phenyl)amino)prop-1-yn-1-yl)-N-(1-methylpiperidin-4-yl)thieno |
| | [3,2-c]pyridin-7-amine; |
| 781 | 4-((3-(3-ethyl-7-((1-methylpiperidin-4-yl)amino)thieno[3,2-c]pyridin-2-yl)prop-2-yn-1-yl)amino)-3-methoxy- |
| | N-methylbenzamide; |
| 782 | 4-((3-(3-ethyl-7-((1-methylpiperidin-4-yl)amino)thieno[3,2-c]pyridin-2-yl)prop-2-yn-1-yl)amino)-3- |
| | methoxybenzenesulfonamide; |
| 783 | 1-(4-((3-(cyclopropylmethyl)-2-(3-((2-methoxy-4-(methylsulfonyl)phenyl)amino)prop-1-yn-1-yl)thieno[3,2- |
| | c]pyridin-7-yl)amino)-3-fluoropiperidin-1-yl)-3-methoxypropan-2-o1; |
| 784 | 4-((3-(3-(cyclopropylmethyl)-7-((3-fluoro-1-(2-hydroxy-3-methoxypropyl)piperidin-4-yl)amino)thieno [3,2- |
| | c]pyridin-2-yl)prop-2-yn-1-yl)amino)-3-methoxy-N-methylbenzamide; |
| 785 | 4-((3-(3-(cyclopropylmethyl)-7-((3-fluoro-1-(2-hydroxy-3-methoxypropyl)piperidin-4-yl)amino)thieno[3,2- |
| | c]pyridin-2-yl)prop-2-yn-1-yl)amino)-3-methoxybenzenesulfonamide; |
| 786 | 1-(3-fluoro-4-((2-(3-((2-methoxy-4-(methylsulfonyl)phenyl)amino)prop-1-yn-1-yl)-3-(2,2,2-trifluoroethyl) |
| | thieno[3,2-c]pyridin-7-yl)amino)piperidin-1-yl)-3-methoxypropan-2-ol; |
| 787 | 4-((3-(7-((3-fluoro-1-(2-hydroxy-3-methoxypropyl)piperidin-4-yl)amino)-3-(2,2,2-trifluoroethyl)thieno[3,2- |
| | c]pyridin-2-yl)prop-2-yn-1-yl)amino)-3-methoxy-N-methylbenzamide; |
| 788 | 4-((3-(7-((3-fluoro-1-(2-hydroxy-3-methoxypropyl)piperidin-4-yl)amino)-3-(2,2,2-trifluoroethyl)thieno[3,2- |
| | c]pyridin-2-yl)prop-2-yn-1-yl)amino)-3-methoxybenzenesulfonamide; |
| 789 | 1-(4-((3-ethyl-2-(3-((2-methoxy-4-(methylsulfonyl)phenyl)amino)prop-1-yn-1-yl)thieno[3,2-c]pyridin-7-yl) |
| | amino)-3-fluoropiperidin-1-yl)-3-methoxypropan-2-ol; |
| 790 | 4-((3-(3-ethyl-7-((3-fluoro-1-(2-hydroxy-3-methoxypropyl)piperidin-4-yl)amino)thieno[3,2-c]pyridin-2-yl) |
| | prop-2-yn-1-yl)amino)-3-methoxy-N-methylbenzamide; |
| 791 | 4-((3-(3-ethyl-7-((3-fluoro-1-(2-hydroxy-3-methoxypropyl)piperidin-4-yl)amino)thieno[3,2-c]pyridin-2-yl) |
| | prop-2-yn-1-yl)amino)-3-methoxybenzenesulfonamide; |
| 792 | 3-(cyclopropylmethyl)-N-(3-fluoro-1-methylpiperidin-4-yl)-2-(3-((2-methoxy-4-(methylsulfonyl)phenyl)amino) |
| | prop-1-yn-1-yl)thieno[3,2-c]pyridin-7-amine; |
| 793 | 4-((3-(3-(cyclopropylmethyl)-7-((3-fluoro-1-methylpiperidin-4-yl)amino)thieno[3,2-c]pyridin-2-yl)prop-2-yn- |
| | 1-yl)amino)-3-methoxy-N-methylbenzamide; |
| 794 | 4-((3-(3-(cyclopropylmethyl)-7-((3-fluoro-1-methylpiperidin-4-yl)amino)thieno[3,2-c]pyridin-2-yl)prop-2-yn- |
| | 1-yl)amino)-3-methoxybenzenesulfonamide; |
| 795 | N-(3-fluoro-1-methylpiperidin-4-yl)-2-(3-((2-methoxy-4-(methylsulfonyl)phenyl)amino)prop-1-yn-1-yl)-3- |
| | (2,2,2-trifluoroethyl)thieno[3,2-c]pyridin-7-amine; |
| 796 | 4-((3-(7-((3-fluoro-1-methylpiperidin-4-yl)amino)-3-(2,2,2-trifluoroethyl)thieno[3,2-c]pyridin-2-yl)prop-2- |
| | yn-1-yl)amino)-3-methoxy-N-methylbenzamide; |
| 797 | 4-((3-(7-((3-fluoro-1-methylpiperidin-4-yl)amino)-3-(2,2,2-trifluoroethyl)thieno[3,2-c]pyridin-2-yl)prop-2- |
| | yn-1-yl)amino)-3-methoxybenzenesulfonamide; |
| 798 | 3-ethyl-N-(3-fluoro-1-methylpiperidin-4-yl)-2-(3-((2-methoxy-4-(methylsulfonyl)phenyl)amino)prop-1-yn- |
| | 1-yl)thieno[3,2-c]pyridin-7-amine; |
| 799 | 4-((3-(3-ethyl-7-((3-fluoro-1-methylpiperidin-4-yl)amino)thieno[3,2-c]pyridin-2-yl)prop-2-yn-1-yl)amino)- |
| | 3-methoxy-N-methylbenzamide; |
| 800 | 4-((3-(3-ethyl-7-((3-fluoro-1-methylpiperidin-4-yl)amino)thieno[3,2-c]pyridin-2-yl)prop-2-yn-1-yl)amino)- |
| | 3-methoxybenzenesulfonamide; |
| 801 | N1-(3-(cyclopropylmethyl)-2-(3-((2-methoxy-4-(methylsulfonyl)phenyl)amino)prop-1-yn-1-yl)thieno[3,2- |
| | c]pyridin-7-yl)-N4,N4-dimethylcyclohexane-1,4-diamine; |
| 802 | 4-((3-(3-(cyclopropylmethyl)-7-((4-(dimethylamino)cyclohexyl)amino)thieno[3,2-c]pyridin-2-yl)prop-2-yn- |
| | 1-yl)amino)-3-methoxy-N-methylbenzamide; |
| 803 | 4-((3-(3-(cyclopropylmethyl)-7-((4-(dimethylamino)cyclohexyl)amino)thieno[3,2-c]pyridin-2-yl)prop-2-yn- |
| | 1-yl)amino)-3-methoxybenzenesulfonamide; |
| 804 | N1-(2-(3-((2-methoxy-4-(methylsulfonyl)phenyl)amino)prop-1-yn-1-yl)-3-(2,2,2-trifluoroethyl)thieno[3,2- |
| | c]pyridin-7-yl)-N4,N4-dimethylcyclohexane-1,4-diamine; |
| 805 | 4-((3-(7-((4-(dimethylamino)cyclohexyl)amino)-3-(2,2,2-trifluoroethyl)thieno[3,2-c]pyridin-2-yl)prop-2-yn- |
| | 1-yl)amino)-3-methoxy-N-methylbenzamide; |
| 806 | 4-((3-(7-((4-(dimethylamino)cyclohexyl)amino)-3-(2,2,2-trifluoroethyl)thieno[3,2-c]pyridin-2-yl)prop-2-yn- |
| | 1-yl)amino)-3-methoxybenzenesulfonamide; |
| 807 | N1-(3-ethyl-2-(3-((2-methoxy-4-(methylsulfonyl)phenyl)amino)prop-1-yn-1-yl)thieno[3,2-c]pyridin-7-yl)- |
| | N4,N4-dimethylcyclohexane-1,4-diamine; |
| 808 | 4-((3-(7-((4-(dimethylamino)cyclohexyl)amino)-3-ethylthieno[3,2-c]pyridin-2-yl)prop-2-yn-1-yl)amino)-3- |
| | methoxy-N-methylbenzamide; |
| 809 | 4-((3-(7-((4-(dimethylamino)cyclohexyl)amino)-3-ethylthieno[3,2-c]pyridin-2-yl)prop-2-yn-1-yl)amino)-3- |
| | methoxybenzenesulfonamide; |
| 810 | 3-(cyclopropylmethyl)-N-(3,3-difluoro-1-methylpiperidin-4-yl)-2-(3-((2-methoxy-4-(methylsulfonyl)phenyl) |
| | amino)prop-1-yn-1-yl)thieno[3,2-c]pyridin-7-amine; |
| 811 | 4-((3-(3-(cyclopropylmethyl)-7-((3,3-difluoro-1-methylpiperidin-4-yl)amino)thieno[3,2-c]pyridin-2-yl)prop- |
| | 2-yn-1-yl)amino)-3-methoxy-N-methylbenzamide; |
| 812 | 4-((3-(3-(cyclopropylmethyl)-7-((3,3-difluoro-1-methylpiperidin-4-yl)amino)thieno[3,2-c]pyridin-2-yl)prop- |
| | 2-yn-1-yl)amino)-3-methoxybenzenesulfonamide; |
| 813 | N-(3,3-difluoro-1-methylpiperidin-4-yl)-2-(3-((2-methoxy-4-(methylsulfonyl)phenyl)amino)prop-1-yn-1-yl)- |
| | 3-(2,2,2-trifluoroethyl)thieno[3,2-c]pyridin-7-amine; |
| 814 | 4-((3-(7-((3,3-difluoro-1-methylpiperidin-4-yl)amino)-3-(2,2,2-trifluoroethyl)thieno[3,2-c]pyridin-2-yl)prop- |
| | 2-yn-1-yl)amino)-3-methoxy-N-methylbenzamide; |
| 815 | 4-((3-(7-((3,3-difluoro-1-methylpiperidin-4-yl)amino)-3-(2,2,2-trifluoroethyl)thieno[3,2-c]pyridin-2-yl)prop- |
| | 2-yn-1-yl)amino)-3-methoxybenzenesulfonamide; |
| 816 | N-(3,3-difluoro-1-methylpiperidin-4-yl)-3-ethyl-2-(3-((2-methoxy-4-(methylsulfonyl)phenyl)amino)prop-1- |
| | yn-1-yl)thieno[3,2-c]pyridin-7-amine; |
| 817 | 4-((3-(7-((3,3-difluoro-1-methylpiperidin-4-yl)amino)-3-ethylthieno[3,2-c]pyridin-2-yl)prop-2-yn-1-yl)amino)- |
| | 3-methoxy-N-methylbenzamide; |
| 818 | 4-((3-(7-((3,3-difluoro-1-methylpiperidin-4-yl)amino)-3-ethylthieno[3,2-c]pyridin-2-yl)prop-2-yn-1-yl)amino)- |
| | 3-methoxybenzenesulfonamide; |
| 819 | 3-(cyclopropylmethyl)-2-(3-((2-methoxy-4-(methylsulfonyl)phenyl)amino)prop-1-yn-1-yl)-N-(1- |
| | methylpiperidin-4-yl)thieno[2,3-c]pyridin-7-amine; |
| 820 | 4-((3-(3-(cyclopropylmethyl)-7-((1-methylpiperidin-4-yl)amino)thieno[2,3-c]pyridin-2-yl)prop-2-yn-1-yl) |
| | amino)-3-methoxy-N-methylbenzamide; |
| 821 | 4-((3-(3-(cyclopropylmethyl)-7-((1-methylpiperidin-4-yl)amino)thieno[2,3-c]pyridin-2-yl)prop-2-yn-1-yl) |
| | amino)-3-methoxybenzenesulfonamide; |
| 822 | 1-(4-((3-(cyclopropylmethyl)-2-(3-((2-methoxy-4-(methylsulfonyl)phenyl)amino)prop-1-yn-1-yl)thieno[2,3- |
| | c]pyridin-7-yl)amino)-3-fluoropiperidin-1-yl)-3-methoxypropan-2-ol; |
| 823 | 4-((3-(3-(cyclopropylmethyl)-7-((3-fluoro-1-(2-hydroxy-3-methoxypropyl)piperidin-4-yl)amino)thieno[2,3- |
| | c]pyridin-2-yl)prop-2-yn-1-yl)amino)-3-methoxy-N-methylbenzamide; |
| 824 | 4-((3-(3-(cyclopropylmethyl)-7-((3-fluoro-1-(2-hydroxy-3-methoxypropyl)piperidin-4-yl)amino)thieno[2,3- |
| | c]pyridin-2-yl)prop-2-yn-1-yl)amino)-3-methoxybenzenesulfonamide; |
| 825 | 3-(cyclopropylmethyl)-N-(3-fluoro-1-methylpiperidin-4-yl)-2-(3-((2-methoxy-4-(methylsulfonyl)phenyl)amino) |
| | prop-1-yn-1-yl)thieno[2,3-c]pyridin-7-amine; |
| 826 | 4-((3-(3-(cyclopropylmethyl)-7-((3-fluoro-1-methylpiperidin-4-yl)amino)thieno[2,3-c]pyridin-2-yl)prop-2-yn- |
| | 1-yl)amino)-3-methoxy-N-methylbenzamide; |
| 827 | 4-((3-(3-(cyclopropylmethyl)-7-((3-fluoro-1-methylpiperidin-4-yl)amino)thieno[2,3-c]pyridin-2-yl)prop-2-yn- |
| | 1-yl)amino)-3-methoxybenzenesulfonamide; |
| 828 | N1-(3-(cyclopropylmethyl)-2-(3-((2-methoxy-4-(methylsulfonyl)phenyl)amino)prop-1-yn-1-yl)thieno[2,3- |
| | c]pyridin-7-yl)-N4,N4-dimethylcyclohexane-1,4-diamine; |
| 829 | 4-((3-(3-(cyclopropylmethyl)-7-((4-(dimethylamino)cyclohexyl)amino)thieno[2,3-c]pyridin-2-yl)prop-2-yn- |
| | 1-yl)amino)-3-methoxy-N-methylbenzamide; |
| 830 | 4-((3-(3-(cyclopropylmethyl)-7-((4-(dimethylamino)cyclohexyl)amino)thieno[2,3-c]pyridin-2-yl)prop-2-yn- |
| | 1-yl)amino)-3-methoxybenzenesulfonamide; |
| 831 | 3-(cyclopropylmethyl)-N-(3,3-difluoro-1-methylpiperidin-4-yl)-2-(3-((2-methoxy-4-(methylsulfonyl)phenyl) |
| | amino)prop-1-yn-1-yl)thieno[2,3-c]pyridin-7-amine; |
| 832 | 4-((3-(3-(cyclopropylmethyl)-7-((3,3-difluoro-1-methylpiperidin-4-yl)amino)thieno[2,3-c]pyridin-2-yl)prop- |
| | 2-yn-1-yl)amino)-3-methoxy-N-methylbenzamide; |
| 833 | 4-((3-(3-(cyclopropylmethyl)-7-((3,3-difluoro-1-methylpiperidin-4-yl)amino)thieno[2,3-c]pyridin-2-yl)prop- |
| | 2-yn-1-yl)amino)-3-methoxybenzenesulfonamide; |
| 834 | 2-(3-((2-methoxy-4-(methylsulfonyl)phenyl)amino)prop-1-yn-1-yl)-N-(1-methylpiperidin-4-yl)-3-(2,2,2- |
| | trifluoroethyl)thieno[2,3-c]pyridin-7-amine; |
| 835 | 3-methoxy-N-methyl-4-((3-(7-((1-methylpiperidin-4-yl)amino)-3-(2,2,2-trifluoroethyl)thieno[2,3-c]pyridin- |
| | 2-yl)prop-2-yn-1-yl)amino)benzamide; |
| 836 | 3-methoxy-4-((3-(7-((1-methylpiperidin-4-yl)amino)-3-(2,2,2-trifluoroethyl)thieno[2,3-c]pyridin-2-yl)prop- |
| | 2-yn-1-yl)amino)benzenesulfonamide; |
| 837 | 1-(3-fluoro-4-((2-(3-((2-methoxy-4-(methylsulfonyl)phenyl)amino)prop-1-yn-1-yl)-3-(2,2,2-trifluoroethyl) |
| | thieno[2,3-c]pyridin-7-yl)amino)piperidin-1-yl)-3-methoxypropan-2-ol; |
| 838 | 4-((3-(7-((3-fluoro-1-(2-hydroxy-3-methoxypropyl)piperidin-4-yl)amino)-3-(2,2,2-trifluoroethyl)thieno [2,3- |
| | c]pyridin-2-yl)prop-2-yn-1-yl)amino)-3-methoxy-N-methylbenzamide; |
| 839 | 4-((3-(7-((3-fluoro-1-(2-hydroxy-3-methoxypropyl)piperidin-4-yl)amino)-3-(2,2,2-trifluoroethyl)thieno[2,3- |
| | c]pyridin-2-yl)prop-2-yn-1-yl)amino)-3-methoxybenzenesulfonamide; |
| 840 | N-(3-fluoro-1-methylpiperidin-4-yl)-2-(3-((2-methoxy-4-(methylsulfonyl)phenyl)amino)prop-1-yn-1-yl)-3- |
| | (2,2,2-trifluoroethyl)thieno[2,3-c]pyridin-7-amine; |
| 841 | 4-((3-(7-((3-fluoro-1-methylpiperidin-4-yl)amino)-3-(2,2,2-trifluoroethyl)thieno[2,3-c]pyridin-2-yl)prop-2- |
| | yn-1-yl)amino)-3-methoxy-N-methylbenzamide; |
| 842 | 4-((3-(7-((3-fluoro-1-methylpiperidin-4-yl)amino)-3-(2,2,2-trifluoroethyl)thieno[2,3-c]pyridin-2-yl)prop-2- |
| | yn-1-yl)amino)-3-methoxybenzenesulfonamide; |
| 843 | N1-(2-(3-((2-methoxy-4-(methylsulfonyl)phenyl)amino)prop-1-yn-1-yl)-3-(2,2,2-trifluoroethyl)thieno[2,3- |
| | c]pyridin-7-yl)-N4,N4-dimethylcyclohexane-1,4-diamine; |
| 844 | 4-((3-(7-((4-(dimethylamino)cyclohexyl)amino)-3-(2,2,2-trifluoroethyl)thieno[2,3-c]pyridin-2-yl)prop-2- |
| | yn-1-yl)amino)-3-methoxy-N-methylbenzamide; |
| 845 | 4-((3-(7-((4-(dimethylamino)cyclohexyl)amino)-3-(2,2,2-trifluoroethyl)thieno[2,3-c]pyridin-2-yl)prop-2- |
| | yn-1-yl)amino)-3-methoxybenzenesulfonamide; |
| 846 | N-(3,3-difluoro-1-methylpiperidin-4-yl)-2-(3-((2-methoxy-4-(methylsulfonyl)phenyl)amino)prop-1-yn-1-yl)- |
| | 3-(2,2,2-trifluoroethyl)thieno[2,3-c]pyridin-7-amine; |
| 847 | 4-((3-(7-((3,3-difluoro-1-methylpiperidin-4-yl)amino)-3-(2,2,2-trifluoroethyl)thieno[2,3-c]pyridin-2-yl) |
| | prop-2-yn-1-yl)amino)-3-methoxy-N-methylbenzamide; |
| 848 | 4-((3-(7-((3,3-difluoro-1-methylpiperidin-4-yl)amino)-3-(2,2,2-trifluoroethyl)thieno[2,3-c]pyridin-2-yl) |
| | prop-2-yn-1-yl)amino)-3-methoxybenzenesulfonamide; |
| 849 | 3-ethyl-2-(3-((2-methoxy-4-(methylsulfonyl)phenyl)amino)prop-1-yn-1-yl)-N-(1-methylpiperidin-4-yl)thieno |
| | [2,3-c]pyridin-7-amine; |
| 850 | 4-((3-(3-ethyl-7-((1-methylpiperidin-4-yl)amino)thieno[2,3-c]pyridin-2-yl)prop-2-yn-1-yl)amino)-3-methoxy- |
| | N-methylbenzamide; |
| 851 | 4-((3-(3-ethyl-7-((1-methylpiperidin-4-yl)amino)thieno[2,3-c]pyridin-2-yl)prop-2-yn-1-yl)amino)-3- |
| | methoxybenzenesulfonamide; |
| 852 | 1-(4-((3-ethyl-2-(3-((2-methoxy-4-(methylsulfonyl)phenyl)amino)prop-1-yn-1-yl)thieno[2,3-c]pyridin-7-yl) |
| | amino)-3-fluoropiperidin-1-yl)-3-methoxypropan-2-ol; |
| 853 | 4-((3-(3-ethyl-7-((3-fluoro-1-(2-hydroxy-3-methoxypropyl)piperidin-4-yl)amino)thieno[2,3-c]pyridin-2-yl) |
| | prop-2-yn-1-yl)amino)-3-methoxy-N-methylbenzamide; |
| 854 | 4-((3-(3-ethyl-7-((3-fluoro-1-(2-hydroxy-3-methoxypropyl)piperidin-4-yl)amino)thieno[2,3-c]pyridin-2-yl) |
| | prop-2-yn-1-yl)amino)-3-methoxybenzenesulfonamide; |
| 855 | 3-ethyl-N-(3-fluoro-1-methylpiperidin-4-yl)-2-(3-((2-methoxy-4-(methylsulfonyl)phenyl)amino)prop-1-yn- |
| | 1-yl)thieno[2,3-c]pyridin-7-amine; |
| 856 | 4-((3-(3-ethyl-7-((3-fluoro-1-methylpiperidin-4-yl)amino)thieno[2,3-c]pyridin-2-yl)prop-2-yn-1-yl)amino)- |
| | 3-methoxy-N-methylbenzamide; |
| 857 | 4-((3-(3-ethyl-7-((3-fluoro-1-methylpiperidin-4-yl)amino)thieno[2,3-c]pyridin-2-yl)prop-2-yn-1-yl)amino)- |
| | 3-methoxybenzenesulfonamide; |
| 858 | N1-(3-ethyl-2-(3-((2-methoxy-4-(methylsulfonyl)phenyl)amino)prop-1-yn-1-yl)thieno[2,3-c]pyridin-7-yl)- |
| | N4,N4-dimethylcyclohexane-1,4-diamine; |
| 859 | 4-((3-(7-((4-(dimethylamino)cyclohexyl)amino)-3-ethylthieno[2,3-c]pyridin-2-yl)prop-2-yn-1-yl)amino)-3- |
| | methoxy-N-methylbenzamide; |
| 860 | 4-((3-(7-((4-(dimethylamino)cyclohexyl)amino)-3-ethylthieno[2,3-c]pyridin-2-yl)prop-2-yn-1-yl)amino)-3- |
| | methoxybenzenesulfonamide; |
| 861 | N-(3,3-difluoro-1-methylpiperidin-4-yl)-3-ethyl-2-(3-((2-methoxy-4-(methylsulfonyl)phenyl)amino)prop-1- |
| | yn-1-yl)thieno[2,3-c]pyridin-7-amine; |
| 862 | 4-((3-(7-((3,3-difluoro-1-methylpiperidin-4-yl)amino)-3-ethylthieno[2,3-c]pyridin-2-yl)prop-2-yn-1-yl)amino)- |
| | 3-methoxy-N-methylbenzamide; |
| 863 | 4-((3-(7-((3,3-difluoro-1-methylpiperidin-4-yl)amino)-3-ethylthieno[2,3-c]pyridin-2-yl)prop-2-yn-1-yl)amino)- |
| | 3-methoxybenzenesulfonamide; |
| 864 | 4-((3-(3-(cyanomethyl)-7-((3,3-difluoro-1-methylpiperidin-4-yl)amino)-1,1-dioxidobenzo[b]thiophen-2-yl) |
| | prop-2-yn-1-yl)amino)-3-methoxybenzenesulfonamide; |
| 865 | 4-((3-(3-(cyanomethyl)-7-((1,1-dioxidotetrahydro-2H-thiopyran-4-yl)amino)-1,1-dioxidobenzo[b]thiophen- |
| | 2-yl)prop-2-yn-1-yl)amino)-3-methoxybenzenesulfonamide; |
| 866 | 4-((3-(3-(cyanomethyl)-7-(((3R,4S)-3-fluoro-1-methylpiperidin-4-yl)amino)-1,1-dioxidobenzo[b]thiophen- |
| | 2-yl)prop-2-yn-1-yl)amino)-3-methoxybenzenesulfonamide; |
| 867 | 4-((3-(3-(cyanomethyl)-7-((3-fluoro-1-methylpiperidin-4-yl)amino)-1,1-dioxidobenzo[b]thiophen-2-yl)prop- |
| | 2-yn-1-yl)amino)-3-methoxybenzenesulfonamide; |
| 868 | 4-((3-(3-(cyanomethyl)-7-((1-(dimethylglycyl)piperidin-4-yl)amino)-1,1-dioxidobenzo[b]thiophen-2-yl)prop- |
| | 2-yn-1-yl)amino)-3-methoxybenzenesulfonamide; |
| 869 | 4-((3-(3-(cyanomethyl)-7-((3-fluoro-1-(2-hydroxy-3-methoxypropyl)piperidin-4-yl)amino)-1,1-dioxidobenzo |
| | [b]thiophen-2-yl)prop-2-yn-1-yl)amino)-3-methoxybenzenesulfonamide; |
| 870 | 4-((3-(3-(cyanomethyl)-7-(((3R,4R)-3-fluoro-1-methylpiperidin-4-yl)amino)-1,1-dioxidobenzo[b]thiophen- |
| | 2-yl)prop-2-yn-1-yl)amino)-3-methoxybenzenesulfonamide; |
| 871 | 4-((3-(3-(cyanomethyl)-7-(((3S,4S)-3-fluoro-1-methylpiperidin-4-yl)amino)-1,1-dioxidobenzo[b]thiophen-2- |
| | yl)prop-2-yn-1-yl)amino)-3-methoxybenzenesulfonamide; |
| 872 | 4-((3-(3-(cyanomethyl)-7-((4-(dimethylamino)cyclohexyl)amino)-1,1-dioxidobenzo[b]thiophen-2-yl)prop-2- |
| | yn-1-yl)amino)-3-methoxybenzenesulfonamide; |
| 873 | 4-((3-(3-(cyanomethyl)-7-((2-(dimethylamino)ethyl)amino)-1,1-dioxidobenzo[b]thiophen-2-yl)prop-2-yn-1- |
| | yl)amino)-3-methoxybenzenesulfonamide; |
| 874 | 4-((3-(3-(cyanomethyl)-7-(((3S,4R)-3-fluoro-1-methylpiperidin-4-yl)amino)-1,1-dioxidobenzo[b]thiophen- |
| | 2-yl)prop-2-yn-1-yl)amino)-3-methoxybenzenesulfonamide; |
| 875 | 4-((3-(3-(cyanomethyl)-7-((1-methylpiperidin-4-yl)amino)-1,1-dioxidobenzo[b]thiophen-2-yl)prop-2-yn-1- |
| | yl)amino)-3-methoxybenzenesulfonamide; |
| 876 | 4-((3-(3-(cyanomethyl)-1,1-dioxido-7-((tetrahydro-2H-pyran-4-yl)amino)benzo[b]thiophen-2-yl)prop-2-yn- |
| | 1-yl)amino)-3-methoxybenzenesulfonamide; |
| 877 | 4-((3-(7-((3,3-difluoro-1-methylpiperidin-4-yl)amino)-1,1-dioxido-3-(thiazol-4-yl)benzo[b]thiophen-2-yl)prop- |
| | 2-yn-1-yl)amino)-3-methoxybenzenesulfonamide; |
| 878 | 4-((3-(7-((1,1-dioxidotetrahydro-2H-thiopyran-4-yl)amino)-1,1-dioxido-3-(thiazol-4-yl)benzo[b]thiophen-2- |
| | yl)prop-2-yn-1-yl)amino)-3-methoxybenzenesulfonamide; |
| 879 | 4-((3-(7-(((3R,4S)-3-fluoro-1-methylpiperidin-4-yl)amino)-1,1-dioxido-3-(thiazol-4-yl)benzo[b]thiophen-2- |
| | yl)prop-2-yn-1-yl)amino)-3-methoxybenzenesulfonamide; |
| 880 | 4-((3-(7-((3-fluoro-1-methylpiperidin-4-yl)amino)-1,1-dioxido-3-(thiazol-4-yl)benzo[b]thiophen-2-yl)prop- |
| | 2-yn-1-yl)amino)-3-methoxybenzenesulfonamide; |
| 881 | 4-((3-(7-((1-(dimethylglycyl)piperidin-4-yl)amino)-1,1-dioxido-3-(thiazol-4-yl)benzo[b]thiophen-2-yl)prop- |
| | 2-yn-1-yl)amino)-3-methoxybenzenesulfonamide; |
| 882 | 4-((3-(7-((3-fluoro-1-(2-hydroxy-3-methoxypropyl)piperidin-4-yl)amino)-1,1-dioxido-3-(thiazol-4-yl)benzo |
| | [b]thiophen-2-yl)prop-2-yn-1-yl)amino)-3-methoxybenzenesulfonamide; |
| 883 | 4-((3-(7-(((3R,4R)-3-fluoro-1-methylpiperidin-4-yl)amino)-1,1-dioxido-3-(thiazol-4-yl)benzo[b]thiophen-2- |
| | yl)prop-2-yn-1-yl)amino)-3-methoxybenzenesulfonamide; |
| 884 | 4-((3-(7-(((3S,4S)-3-fluoro-1-methylpiperidin-4-yl)amino)-1,1-dioxido-3-(thiazol-4-yl)benzo[b]thiophen-2- |
| | yl)prop-2-yn-1-yl)amino)-3-methoxybenzenesulfonamide; |
| 885 | 4-((3-(7-((4-(dimethylamino)cyclohexyl)amino)-1,1-dioxido-3-(thiazol-4-yl)benzo[b]thiophen-2-yl)prop-2- |
| | yn-1-yl)amino)-3-methoxybenzenesulfonamide; |
| 886 | 4-((3-(7-((2-(dimethylamino)ethyl)amino)-1,1-dioxido-3-(thiazol-4-yl)benzo[b]thiophen-2-yl)prop-2-yn-1- |
| | yl)amino)-3-methoxybenzenesulfonamide; |
| 887 | 4-((3-(7-(((3S,4R)-3-fluoro-1-methylpiperidin-4-yl)amino)-1,1-dioxido-3-(thiazol-4-yl)benzo[b]thiophen-2- |
| | yl)prop-2-yn-1-yl)amino)-3-methoxybenzenesulfonamide; |
| 888 | 3-methoxy-4-((3-(7-((1-methylpiperidin-4-yl)amino)-1,1-dioxido-3-(thiazol-4-yl)benzo[b]thiophen-2-yl)prop- |
| | 2-yn-1-yl)amino)benzenesulfonamide; |
| 889 | 4-((3-(1,1-dioxido-7-((tetrahydro-2H-pyran-4-yl)amino)-3-(thiazol-4-yl)benzo[b]thiophen-2-yl)prop-2-yn-1- |
| | yl)amino)-3-methoxybenzenesulfonamide; |
| 890 | 4-((3-(7-((3,3-difluoro-1-methylpiperidin-4-yl)amino)-1,1-dioxido-3-(2,2,2-trifluoroethyl)benzo[b]thiophen- |
| | 2-yl)prop-2-yn-1-yl)amino)-3-methoxybenzenesulfonamide; |
| 891 | 4-((3-(7-((1,1-dioxidotetrahydro-2H-thiopyran-4-yl)amino)-1,1-dioxido-3-(2,2,2-trifluoroethyl)benzo[b]thiophen- |
| | 2-yl)prop-2-yn-1-yl)amino)-3-methoxybenzenesulfonamide; |
| 892 | 4-((3-(7-(((3R,4S)-3-fluoro-1-methylpiperidin-4-yl)amino)-1,1-dioxido-3-(2,2,2-trifluoroethyl)benzo[b]thiophen- |
| | 2-yl)prop-2-yn-1-yl)amino)-3-methoxybenzenesulfonamide; |
| 893 | 4-((3-(7-((3-fluoro-1-methylpiperidin-4-yl)amino)-1,1-dioxido-3-(2,2,2-trifluoroethyl)benzo[b]thiophen-2- |
| | yl)prop-2-yn-1-yl)amino)-3-methoxybenzenesulfonamide; |
| 894 | 4-((3-(7-((1-(dimethylglycyl)piperidin-4-yl)amino)-1,1-dioxido-3-(2,2,2-trifluoroethyl)benzo[b]thiophen-2- |
| | yl)prop-2-yn-1-yl)amino)-3-methoxybenzenesulfonamide; |
| 895 | 4-((3-(7-((3-fluoro-1-(2-hydroxy-3-methoxypropyl)piperidin-4-yl)amino)-1,1-dioxido-3-(2,2,2- |
| | trifluoroethyl)benzo[b]thiophen-2-yl)prop-2-yn-1-yl)amino)-3-methoxybenzenesulfonamide; |
| 896 | 4-((3-(7-(((3R,4R)-3-fluoro-1-methylpiperidin-4-yl)amino)-1,1-dioxido-3-(2,2,2-trifluoroethyl)benzo[b]thiophen- |
| | 2-yl)prop-2-yn-1-yl)amino)-3-methoxybenzenesulfonamide; |
| 897 | 4-((3-(7-(((3S,4S)-3-fluoro-1-methylpiperidin-4-yl)amino)-1,1-dioxido-3-(2,2,2-trifluoroethyl)benzo[b]thiophen- |
| | 2-yl)prop-2-yn-1-yl)amino)-3-methoxybenzenesulfonamide; |
| 898 | 4-((3-(7-((4-(dimethylamino)cyclohexyl)amino)-1,1-dioxido-3-(2,2,2-trifluoroethyl)benzo[b]thiophen-2-yl) |
| | prop-2-yn-1-yl)amino)-3-methoxybenzenesulfonamide; |
| 899 | 4-((3-(7-((2-(dimethylamino)ethyl)amino)-1,1-dioxido-3-(2,2,2-trifluoroethyl)benzo[b]thiophen-2-yl)prop- |
| | 2-yn-1-yl)amino)-3-methoxybenzenesulfonamide; |
| 900 | 4-((3-(7-(((3S,4R)-3-fluoro-1-methylpiperidin-4-yl)amino)-1,1-dioxido-3-(2,2,2-trifluoroethyl)benzo[b]thiophen- |
| | 2-yl)prop-2-yn-1-yl)amino)-3-methoxybenzenesulfonamide; |
| 901 | 3-methoxy-4-((3-(7-((1-methylpiperidin-4-yl)amino)-1,1-dioxido-3-(2,2,2-trifluoroethyl)benzo[b]thiophen- |
| | 2-yl)prop-2-yn-1-yl)amino)benzenesulfonamide; |
| 902 | 4-((3-(1,1-dioxido-7-((tetrahydro-2H-pyran-4-yl)amino)-3-(2,2,2-trifluoroethyl)benzo[b]thiophen-2-yl)prop- |
| | 2-yn-1-yl)amino)-3-methoxybenzenesulfonamide; |
| 903 | 4-((3-(3-(cyclopropylmethyl)-7-((3,3-difluoro-1-methylpiperidin-4-yl)amino)-1,1-dioxidobenzo[b]thiophen- |
| | 2-yl)prop-2-yn-1-yl)amino)-3-methoxybenzenesulfonamide; |
| 904 | 4-((3-(3-(cyclopropylmethyl)-7-((1,1-dioxidotetrahydro-2H-thiopyran-4-yl)amino)-1,1-dioxidobenzo[b]thiophen- |
| | 2-yl)prop-2-yn-1-yl)amino)-3-methoxybenzenesulfonamide; |
| 905 | 4-((3-(3-(cyclopropylmethyl)-7-(((3R,4S)-3-fluoro-1-methylpiperidin-4-yl)amino)-1,1-dioxidobenzo[b]thiophen- |
| | 2-yl)prop-2-yn-1-yl)amino)-3-methoxybenzenesulfonamide; |
| 906 | 4-((3-(3-(cyclopropylmethyl)-7-((3-fluoro-1-methylpiperidin-4-yl)amino)-1,1-dioxidobenzo[b]thiophen-2- |
| | yl)prop-2-yn-1-yl)amino)-3-methoxybenzenesulfonamide; |
| 907 | 4-((3-(3-(cyclopropylmethyl)-7-((1-(dimethylglycyl)piperidin-4-yl)amino)-1,1-dioxidobenzo[b]thiophen-2- |
| | yl)prop-2-yn-1-yl)amino)-3-methoxybenzenesulfonamide; |
| 908 | 4-((3-(3-(cyclopropylmethyl)-7-((3-fluoro-1-(2-hydroxy-3-methoxypropyl)piperidin-4-yl)amino)-1,1- |
| | dioxidobenzo[b]thiophen-2-yl)prop-2-yn-1-yl)amino)-3-methoxybenzenesulfonamide; |
| 909 | 4-((3-(3-(cyclopropylmethyl)-7-(((3R,4R)-3-fluoro-1-methylpiperidin-4-yl)amino)-1,1-dioxidobenzo[b]thiophen- |
| | 2-yl)prop-2-yn-1-yl)amino)-3-methoxybenzenesulfonamide; |
| 910 | 4-((3-(3-(cyclopropylmethyl)-7-(((3S,4S)-3-fluoro-1-methylpiperidin-4-yl)amino)-1,1-dioxidobenzo[b]thiophen- |
| | 2-yl)prop-2-yn-1-yl)amino)-3-methoxybenzenesulfonamide; |
| 911 | 4-((3-(3-(cyclopropylmethyl)-7-((4-(dimethylamino)cyclohexyl)amino)-1,1-dioxidobenzo[b]thiophen-2-yl) |
| | prop-2-yn-1-yl)amino)-3-methoxybenzenesulfonamide; |
| 912 | 4-((3-(3-(cyclopropylmethyl)-7-((2-(dimethylamino)ethyl)amino)-1,1-dioxidobenzo[b]thiophen-2-yl)prop-2- |
| | yn-1-yl)amino)-3-methoxybenzenesulfonamide; |
| 913 | 4-((3-(3-(cyclopropylmethyl)-7-(((3S,4R)-3-fluoro-1-methylpiperidin-4-yl)amino)-1,1-dioxidobenzo[b]thiophen- |
| | 2-yl)prop-2-yn-1-yl)amino)-3-methoxybenzenesulfonamide; |
| 914 | 4-((3-(3-(cyclopropylmethyl)-7-((1-methylpiperidin-4-yl)amino)-1,1-dioxidobenzo[b]thiophen-2-yl)prop-2- |
| | yn-1-yl)amino)-3-methoxybenzenesulfonamide; |
| 915 | 4-((3-(3-(cyclopropylmethyl)-1,1-dioxido-7-((tetrahydro-2H-pyran-4-yl)amino)benzo[b]thiophen-2-yl)prop- |
| | 2-yn-1-yl)amino)-3-methoxybenzenesulfonamide; |
| 916 | 4-((3-(7-((3,3-difluoro-1-methylpiperidin-4-yl)amino)-3-(2,2-difluoroethyl)-1,1-dioxidobenzo[b]thiophen-2- |
| | yl)prop-2-yn-1-yl)amino)-3-methoxybenzenesulfonamide; |
| 917 | 4-((3-(3-(2,2-difluoroethyl)-7-((1,1-dioxidotetrahydro-2H-thiopyran-4-yl)amino)-1,1-dioxidobenzo[b]thiophen- |
| | 2-yl)prop-2-yn-1-yl)amino)-3-methoxybenzenesulfonamide; |
| 918 | 4-((3-(3-(2,2-difluoroethyl)-7-(((3R,4S)-3-fluoro-1-methylpiperidin-4-yl)amino)-1,1-dioxidobenzo[b]thiophen- |
| | 2-yl)prop-2-yn-1-yl)amino)-3-methoxybenzenesulfonamide; |
| 919 | 4-((3-(3-(2,2-difluoroethyl)-7-((3-fluoro-1-methylpiperidin-4-yl)amino)-1,1-dioxidobenzo[b]thiophen-2-yl) |
| | prop-2-yn-1-yl)amino)-3-methoxybenzenesulfonamide; |
| 920 | 4-((3-(3-(2,2-difluoroethyl)-7-((1-(dimethylglycyl)piperidin-4-yl)amino)-1,1-dioxidobenzo[b]thiophen-2-yl) |
| | prop-2-yn-1-yl)amino)-3-methoxybenzenesulfonamide; |
| 921 | 4-((3-(3-(2,2-difluoroethyl)-7-((3-fluoro-1-(2-hydroxy-3-methoxypropyl)piperidin-4-yl)amino)-1,1- |
| | dioxidobenzo[b]thiophen-2-yl)prop-2-yn-1-yl)amino)-3-methoxybenzenesulfonamide; |
| 922 | 4-((3-(3-(2,2-difluoroethyl)-7-(((3R,4R)-3-fluoro-1-methylpiperidin-4-yl)amino)-1,1-dioxidobenzo[b]thiophen- |
| | 2-yl)prop-2-yn-1-yl)amino)-3-methoxybenzenesulfonamide; |
| 923 | 4-((3-(3-(2,2-difluoroethyl)-7-(((3S,4S)-3-fluoro-1-methylpiperidin-4-yl)amino)-1,1-dioxidobenzo[b]thiophen- |
| | 2-yl)prop-2-yn-1-yl)amino)-3-methoxybenzenesulfonamide; |
| 924 | 4-((3-(3-(2,2-difluoroethyl)-7-((4-(dimethylamino)cyclohexyl)amino)-1,1-dioxidobenzo[b]thiophen-2-yl)prop- |
| | 2-yn-1-yl)amino)-3-methoxybenzenesulfonamide; |
| 925 | 4-((3-(3-(2,2-difluoroethyl)-7-((2-(dimethylamino)ethyl)amino)-1,1-dioxidobenzo[b]thiophen-2-yl)prop-2-yn- |
| | 1-yl)amino)-3-methoxybenzenesulfonamide; |
| 926 | 4-((3-(3-(2,2-difluoroethyl)-7-(((3S,4R)-3-fluoro-1-methylpiperidin-4-yl)amino)-1,1-dioxidobenzo[b]thiophen- |
| | 2-yl)prop-2-yn-1-yl)amino)-3-methoxybenzenesulfonamide; |
| 927 | 4-((3-(3-(2,2-difluoroethyl)-7-((1-methylpiperidin-4-yl)amino)-1,1-dioxidobenzo[b]thiophen-2-yl)prop-2-yn- |
| | 1-yl)amino)-3-methoxybenzenesulfonamide; |
| 928 | 4-((3-(3-(2,2-difluoroethyl)-1,1-dioxido-7-((tetrahydro-2H-pyran-4-yl)amino)benzo[b]thiophen-2-yl)prop-2- |
| | yn-1-yl)amino)-3-methoxybenzenesulfonamide; |
| 929 | 4-((3-(7-((3,3-difluoro-1-methylpiperidin-4-yl)amino)-1,1-dioxido-3-(1H-pyrrol-1-yl)benzo[b]thiophen-2- |
| | yl)prop-2-yn-1-yl)amino)-3-methoxybenzenesulfonamide; |
| 930 | 4-((3-(7-((1,1-dioxidotetrahydro-2H-thiopyran-4-yl)amino)-1,1-dioxido-3-(1H-pyrrol-1-yl)benzo[b]thiophen- |
| | 2-yl)prop-2-yn-1-yl)amino)-3-methoxybenzenesulfonamide; |
| 931 | 4-((3-(7-(((3R,4S)-3-fluoro-1-methylpiperidin-4-yl)amino)-1,1-dioxido-3-(1H-pyrrol-1-yl)benzo[b]thiophen- |
| | 2-yl)prop-2-yn-1-yl)amino)-3-methoxybenzenesulfonamide; |
| 932 | 4-((3-(7-((3-fluoro-1-methylpiperidin-4-yl)amino)-1,1-dioxido-3-(1H-pyrrol-1-yl)benzo[b]thiophen-2-yl)prop- |
| | 2-yn-1-yl)amino)-3-methoxybenzenesulfonamide; |
| 933 | 4-((3-(7-((1-(dimethylglycyl)piperidin-4-yl)amino)-1,1-dioxido-3-(1H-pyrrol-1-yl)benzo[b]thiophen-2-yl)prop- |
| | 2-yn-1-yl)amino)-3-methoxybenzenesulfonamide; |
| 934 | 4-((3-(7-((3-fluoro-1-(2-hydroxy-3-methoxypropyl)piperidin-4-yl)amino)-1,1-dioxido-3-(1H-pyrrol-1- |
| | yl)benzo[b]thiophen-2-yl)prop-2-yn-1-yl)amino)-3-methoxybenzenesulfonamide; |
| 935 | 4-((3-(7-(((3R,4R)-3-fluoro-1-methylpiperidin-4-yl)amino)-1,1-dioxido-3-(1H-pyrrol-1-yl)benzo[b]thiophen- |
| | 2-yl)prop-2-yn-1-yl)amino)-3-methoxybenzenesulfonamide; |
| 936 | 4-((3-(7-(((3S,4S)-3-fluoro-1-methylpiperidin-4-yl)amino)-1,1-dioxido-3-(1H-pyrrol-1-yl)benzo[b]thiophen- |
| | 2-yl)prop-2-yn-1-yl)amino)-3-methoxybenzenesulfonamide; |
| 937 | 4-((3-(7-((4-(dimethylamino)cyclohexyl)amino)-1,1-dioxido-3-(1H-pyrrol-1-yl)benzo[b]thiophen-2-yl)prop- |
| | 2-yn-1-yl)amino)-3-methoxybenzenesulfonamide; |
| 938 | 4-((3-(7-((2-(dimethylamino)ethyl)amino)-1,1-dioxido-3-(1H-pyrrol-1-yl)benzo[b]thiophen-2-yl)prop-2-yn- |
| | 1-yl)amino)-3-methoxybenzenesulfonamide; |
| 939 | 4-((3-(7-(((3S,4R)-3-fluoro-1-methylpiperidin-4-yl)amino)-1,1-dioxido-3-(1H-pyrrol-1-yl)benzo[b]thiophen- |
| | 2-yl)prop-2-yn-1-yl)amino)-3-methoxybenzenesulfonamide; |
| 940 | 3-methoxy-4-((3-(7-((1-methylpiperidin-4-yl)amino)-1,1-dioxido-3-(1H-pyrrol-1-yl)benzo[b]thiophen-2-yl) |
| | prop-2-yn-1-yl)amino)benzenesulfonamide; |
| 941 | 4-((3-(1,1-dioxido-3-(1H-pyrrol-1-yl)-7-((tetrahydro-2H-pyran-4-yl)amino)benzo[b]thiophen-2-yl)prop-2-yn- |
| | 1-yl)amino)-3-methoxybenzenesulfonamide; |
| 942 | 4-((3-(7-((3,3-difluoro-1-methylpiperidin-4-yl)amino)-3-ethyl-1,1-dioxidobenzo[b]thiophen-2-yl)prop-2-yn- |
| | 1-yl)amino)-3-methoxybenzenesulfonamide; |
| 943 | 4-((3-(7-((1,1-dioxidotetrahydro-2H-thiopyran-4-yl)amino)-3-ethyl-1,1-dioxidobenzo[b]thiophen-2-yl)prop- |
| | 2-yn-1-yl)amino)-3-methoxybenzenesulfonamide; |
| 944 | 4-((3-(3-ethyl-7-(((3R,4S)-3-fluoro-1-methylpiperidin-4-yl)amino)-1,1-dioxidobenzo[b]thiophen-2-yl)prop- |
| | 2-yn-1-yl)amino)-3-methoxybenzenesulfonamide; |
| 945 | 4-((3-(3-ethyl-7-((3-fluoro-1-methylpiperidin-4-yl)amino)-1,1-dioxidobenzo[b]thiophen-2-yl)prop-2-yn-1- |
| | yl)amino)-3-methoxybenzenesulfonamide; |
| 946 | 4-((3-(7-((1-(dimethylglycyl)piperidin-4-yl)amino)-3-ethyl-1,1-dioxidobenzo[b]thiophen-2-yl)prop-2-yn-1- |
| | yl)amino)-3-methoxybenzenesulfonamide; |
| 947 | 4-((3-(3-ethyl-7-((3-fluoro-1-(2-hydroxy-3-methoxypropyl)piperidin-4-yl)amino)-1,1-dioxidobenzo[b]thiophen- |
| | 2-yl)prop-2-yn-1-yl)amino)-3-methoxybenzenesulfonamide; |
| 948 | 4-((3-(3-ethyl-7-(((3R,4R)-3-fluoro-1-methylpiperidin-4-yl)amino)-1,1-dioxidobenzo[b]thiophen-2-yl)prop- |
| | 2-yn-1-yl)amino)-3-methoxybenzenesulfonamide; |
| 949 | 4-((3-(3-ethyl-7-(((3S,4S)-3-fluoro-1-methylpiperidin-4-yl)amino)-1,1-dioxidobenzo[b]thiophen-2-yl)prop- |
| | 2-yn-1-yl)amino)-3-methoxybenzenesulfonamide; |
| 950 | 4-((3-(7-((4-(dimethylamino)cyclohexyl)amino)-3-ethyl-1,1-dioxidobenzo[b]thiophen-2-yl)prop-2-yn-1-yl) |
| | amino)-3-methoxybenzenesulfonamide; |
| 951 | 4-((3-(7-((2-(dimethylamino)ethyl)amino)-3-ethyl-1,1-dioxidobenzo[b]thiophen-2-yl)prop-2-yn-1-yl)amino)- |
| | 3-methoxybenzenesulfonamide; |
| 952 | 4-((3-(3-ethyl-7-(((3S,4R)-3-fluoro-1-methylpiperidin-4-yl)amino)-1,1-dioxidobenzo[b]thiophen-2-yl)prop- |
| | 2-yn-1-yl)amino)-3-methoxybenzenesulfonamide; |
| 953 | 4-((3-(3-ethyl-7-((1-methylpiperidin-4-yl)amino)-1,1-dioxidobenzo[b]thiophen-2-yl)prop-2-yn-1-yl)amino)- |
| | 3-methoxybenzenesulfonamide; |
| 954 | 4-((3-(3-ethyl-1,1-dioxido-7-((tetrahydro-2H-pyran-4-yl)amino)benzo[b]thiophen-2-yl)prop-2-yn-1-yl)amino)- |
| | 3-methoxybenzenesulfonamide; |
| 955 | 2-(7-((3,3-difluoro-1-methylpiperidin-4-yl)amino)-2-(3-((5-fluoro-2-methoxy-4-(methylsulfonyl)phenyl) |
| | amino)prop-1-yn-1-yl)-1,1-dioxidobenzo[b]thiophen-3-yl)acetonitrile; |
| 956 | 2-(7-((1,1-dioxidotetrahydro-2H-thiopyran-4-yl)amino)-2-(3-((5-fluoro-2-methoxy-4-(methylsulfonyl)phenyl) |
| | amino)prop-1-yn-1-yl)-1,1-dioxidobenzo[b]thiophen-3-yl)acetonitrile; |
| 957 | 2-(7-(((3R,4S)-3-fluoro-1-methylpiperidin-4-yl)amino)-2-(3-((5-fluoro-2-methoxy-4-(methylsulfonyl)phenyl) |
| | amino)prop-1-yn-1-yl)-1,1-dioxidobenzo[b]thiophen-3-yl)acetonitrile; |
| 958 | 2-(7-((3-fluoro-1-methylpiperidin-4-yl)amino)-2-(3-((5-fluoro-2-methoxy-4-(methylsulfonyl)phenyl)amino) |
| | prop-1-yn-1-yl)-1,1-dioxidobenzo[b]thiophen-3-yl)acetonitrile; |
| 959 | 2-(7-((1-(dimethylglycyl)piperidin-4-yl)amino)-2-(3-((5-fluoro-2-methoxy-4-(methylsulfonyl)phenyl)amino) |
| | prop-1-yn-1-yl)-1,1-dioxidobenzo[b]thiophen-3-yl)acetonitrile; |
| 960 | 2-(7-((3-fluoro-1-(2-hydroxy-3-methoxypropyl)piperidin-4-yl)amino)-2-(3-((5-fluoro-2-methoxy-4- |
| | (methylsulfonyl)phenyl)amino)prop-1-yn-1-yl)-1,1-dioxidobenzo[b]thiophen-3-yl)acetonitrile; |
| 961 | 2-(7-(((3R,4R)-3-fluoro-1-methylpiperidin-4-yl)amino)-2-(3-((5-fluoro-2-methoxy-4-(methylsulfonyl)phenyl) |
| | amino)prop-1-yn-1-yl)-1,1-dioxidobenzo[b]thiophen-3-yl)acetonitrile; |
| 962 | 2-(7-(((3S,4S)-3-fluoro-1-methylpiperidin-4-yl)amino)-2-(3-((5-fluoro-2-methoxy-4-(methylsulfonyl)phenyl) |
| | amino)prop-1-yn-1-yl)-1,1-dioxidobenzo[b]thiophen-3-yl)acetonitrile; |
| 963 | 2-(7-((4-(dimethylamino)cyclohexyl)amino)-2-(3-((5-fluoro-2-methoxy-4-(methylsulfonyl)phenyl)amino)prop- |
| | 1-yn-1-yl)-1,1-dioxidobenzo[b]thiophen-3-yl)acetonitrile; |
| 964 | 2-(7-((2-(dimethylamino)ethyl)amino)-2-(3-((5-fluoro-2-methoxy-4-(methylsulfonyl)phenyl)amino)prop-1- |
| | yn-1-yl)-1,1-dioxidobenzo[b]thiophen-3-yl)acetonitrile; |
| 965 | 2-(7-(((3S,4R)-3-fluoro-1-methylpiperidin-4-yl)amino)-2-(3-((5-fluoro-2-methoxy-4-(methylsulfonyl)phenyl) |
| | amino)prop-1-yn-1-yl)-1,1-dioxidobenzo[b]thiophen-3-yl)acetonitrile; |
| 966 | 2-(2-(3-((5-fluoro-2-methoxy-4-(methylsulfonyl)phenyl)amino)prop-1-yn-1-yl)-7-((1-methylpiperidin-4-yl) |
| | amino)-1,1-dioxidobenzo[b]thiophen-3-yl)acetonitrile; |
| 967 | 2-(2-(3-((5-fluoro-2-methoxy-4-(methylsulfonyl)phenyl)amino)prop-1-yn-1-yl)-1,1-dioxido-7-((tetrahydro- |
| | 2H-pyran-4-yl)amino)benzo[b]thiophen-3-yl)acetonitrile; |
| 968 | 7-((3,3-difluoro-1-methylpiperidin-4-yl)amino)-2-(3-((5-fluoro-2-methoxy-4-(methylsulfonyl)phenyl)amino) |
| | prop-1-yn-1-yl)-3-(thiazol-4-yl)benzo[b]thiophene 1,1-dioxide; |
| 969 | 7-((1,1-dioxidotetrahydro-2H-thiopyran-4-yl)amino)-2-(3-((5-fluoro-2-methoxy-4-(methylsulfonyl)phenyl) |
| | amino)prop-1-yn-1-yl)-3-(thiazol-4-yl)benzo[b]thiophene 1,1-dioxide; |
| 970 | 7-(((3R,4S)-3-fluoro-1-methylpiperidin-4-yl)amino)-2-(3-((5-fluoro-2-methoxy-4-(methylsulfonyl)phenyl) |
| | amino)prop-1-yn-1-yl)-3-(thiazol-4-yl)benzo[b]thiophene 1,1-dioxide; |
| 971 | 7-((3-fluoro-1-methylpiperidin-4-yl)amino)-2-(3-((5-fluoro-2-methoxy-4-(methylsulfonyl)phenyl)amino)prop- |
| | 1-yn-1-yl)-3-(thiazol-4-yl)benzo[b]thiophene 1,1-dioxide; |
| 972 | 2-(dimethylamino)-1-(4-((2-(3-((5-fluoro-2-methoxy-4-(methylsulfonyl)phenyl)amino)prop-1-yn-1-yl)-1,1- |
| | dioxido-3-(thiazol-4-yl)benzo[b]thiophen-7-yl)amino)piperidin-1-yl)ethan-1-one; |
| 973 | 7-((3-fluoro-1-(2-hydroxy-3-methoxypropyl)piperidin-4-yl)amino)-2-(3-((5-fluoro-2-methoxy-4- |
| | (methylsulfonyl)phenyl)amino)prop-1-yn-1-yl)-3-(thiazol-4-yl)benzo[b]thiophene 1,1-dioxide; |
| 974 | 7-(((3R,4R)-3-fluoro-1-methylpiperidin-4-yl)amino)-2-(3-((5-fluoro-2-methoxy-4-(methylsulfonyl)phenyl) |
| | amino)prop-1-yn-1-yl)-3-(thiazol-4-yl)benzo[b]thiophene 1,1-dioxide; |
| 975 | 7-(((3S,4S)-3-fluoro-1-methylpiperidin-4-yl)amino)-2-(3-((5-fluoro-2-methoxy-4-(methylsulfonyl)phenyl) |
| | amino)prop-1-yn-1-yl)-3-(thiazol-4-yl)benzo[b]thiophene 1,1-dioxide; |
| 976 | 7-((4-(dimethylamino)cyclohexyl)amino)-2-(3-((5-fluoro-2-methoxy-4-(methylsulfonyl)phenyl)amino)prop- |
| | 1-yn-1-yl)-3-(thiazol-4-yl)benzo[b]thiophene 1,1-dioxide; |
| 977 | 7-((2-(dimethylamino)ethyl)amino)-2-(3-((5-fluoro-2-methoxy-4-(methylsulfonyl)phenyl)amino)prop-1-yn- |
| | 1-yl)-3-(thiazol-4-yl)benzo[b]thiophene 1,1-dioxide; |
| 978 | 7-(((3S,4R)-3-fluoro-1-methylpiperidin-4-yl)amino)-2-(3-((5-fluoro-2-methoxy-4-(methylsulfonyl)phenyl) |
| | amino)prop-1-yn-1-yl)-3-(thiazol-4-yl)benzo[b]thiophene 1,1-dioxide; |
| 979 | 2-(3-((5-fluoro-2-methoxy-4-(methylsulfonyl)phenyl)amino)prop-1-yn-1-yl)-7-((1-methylpiperidin-4-yl) |
| | amino)-3-(thiazol-4-yl)benzo[b]thiophene 1,1-dioxide; |
| 980 | 2-(3-((5-fluoro-2-methoxy-4-(methylsulfonyl)phenyl)amino)prop-1-yn-1-yl)-7-((tetrahydro-2H-pyran-4-yl) |
| | amino)-3-(thiazol-4-yl)benzo[b]thiophene 1,1-dioxide; |
| 981 | 7-((3,3-difluoro-1-methylpiperidin-4-yl)amino)-2-(3-((5-fluoro-2-methoxy-4-(methylsulfonyl)phenyl)amino) |
| | prop-1-yn-1-yl)-3-(2,2,2-trifluoroethyl)benzo[b]thiophene 1,1-dioxide; |
| 982 | 7-((1,1-dioxidotetrahydro-2H-thiopyran-4-yl)amino)-2-(3-((5-fluoro-2-methoxy-4-(methylsulfonyl)phenyl) |
| | amino)prop-1-yn-1-yl)-3-(2,2,2-trifluoroethyl)benzo[b]thiophene 1,1-dioxide; |
| 983 | 7-(((3R,4S)-3-fluoro-1-methylpiperidin-4-yl)amino)-2-(3-((5-fluoro-2-methoxy-4-(methylsulfonyl)phenyl) |
| | amino)prop-1-yn-1-yl)-3-(2,2,2-trifluoroethyl)benzo[b]thiophene 1,1-dioxide; |
| 984 | 7-((3-fluoro-1-methylpiperidin-4-yl)amino)-2-(3-((5-fluoro-2-methoxy-4-(methylsulfonyl)phenyl)amino)prop- |
| | 1-yn-1-yl)-3-(2,2,2-trifluoroethyl)benzo[b]thiophene 1,1-dioxide; |
| 985 | 2-(dimethylamino)-1-(4-((2-(3-((5-fluoro-2-methoxy-4-(methylsulfonyl)phenyl)amino)prop-1-yn-1-yl)-1,1- |
| | dioxido-3-(2,2,2-trifluoroethyl)benzo[b]thiophen-7-yl)amino)piperidin-1-yl)ethan-1-one; |
| 986 | 7-((3-fluoro-1-(2-hydroxy-3-methoxypropyl)piperidin-4-yl)amino)-2-(3-((5-fluoro-2-methoxy-4- |
| | (methylsulfonyl)phenyl)amino)prop-1-yn-1-yl)-3-(2,2,2-trifluoroethyl)benzo[b]thiophene 1,1-dioxide; |
| 987 | 7-(((3R,4R)-3-fluoro-1-methylpiperidin-4-yl)amino)-2-(3-((5-fluoro-2-methoxy-4-(methylsulfonyl)phenyl) |
| | amino)prop-1-yn-1-yl)-3-(2,2,2-trifluoroethyl)benzo[b]thiophene 1,1-dioxide; |
| 988 | 7-(((3S,4S)-3-fluoro-1-methylpiperidin-4-yl)amino)-2-(3-((5-fluoro-2-methoxy-4-(methylsulfonyl)phenyl) |
| | amino)prop-1-yn-1-yl)-3-(2,2,2-trifluoroethyl)benzo[b]thiophene 1,1-dioxide; |
| 989 | 7-((4-(dimethylamino)cyclohexyl)amino)-2-(3-((5-fluoro-2-methoxy-4-(methylsulfonyl)phenyl)amino)prop- |
| | 1-yn-1-yl)-3-(2,2,2-trifluoroethyl)benzo[b]thiophene 1,1-dioxide; |
| 990 | 7-((2-(dimethylamino)ethyl)amino)-2-(3-((5-fluoro-2-methoxy-4-(methylsulfonyl)phenyl)amino)prop-1-yn- |
| | 1-yl)-3-(2,2,2-trifluoroethyl)benzo[b]thiophene 1,1-dioxide; |
| 991 | 7-(((3S,4R)-3-fluoro-1-methylpiperidin-4-yl)amino)-2-(3-((5-fluoro-2-methoxy-4-(methylsulfonyl)phenyl) |
| | amino)prop-1-yn-1-yl)-3-(2,2,2-trifluoroethyl)benzo[b]thiophene 1,1-dioxide; |
| 992 | 2-(3-((5-fluoro-2-methoxy-4-(methylsulfonyl)phenyl)amino)prop-1-yn-1-yl)-7-((1-methylpiperidin-4-yl)amino)- |
| | 3-(2,2,2-trifluoroethyl)benzo[b]thiophene 1,1-dioxide; |
| 993 | 2-(3-((5-fluoro-2-methoxy-4-(methylsulfonyl)phenyl)amino)prop-1-yn-1-yl)-7-((tetrahydro-2H-pyran-4-yl) |
| | amino)-3-(2,2,2-trifluoroethyl)benzo[b]thiophene 1,1-dioxide; |
| 994 | 3-(cyclopropylmethyl)-7-((3,3-difluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1,1-dioxide; |
| 995 | 3-(cyclopropylmethyl)-7-((1,1-dioxidotetrahydro-2H-thiopyran-4-yl)amino)-2-(3-((5-fluoro-2-methoxy-4- |
| | (methylsulfonyl)phenyl)amino)prop-1-yn-1-yl)benzo[b]thiophene 1,1-dioxide; |
| 996 | 3-(cyclopropylmethyl)-7-(((3R,4S)-3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1,1-dioxide; |
| 997 | 3-(cyclopropylmethyl)-7-((3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1,1-dioxide; |
| 998 | 1-(4-((3-(cyclopropylmethyl)-2-(3-((5-fluoro-2-methoxy-4-(methylsulfonyl)phenyl)amino)prop-1-yn-1-yl)- |
| | 1,1-dioxidobenzo[b]thiophen-7-yl)amino)piperidin-1-yl)-2-(dimethylamino)ethan-1-one; |
| 999 | 3-(cyclopropylmethyl)-7-((3-fluoro-1-(2-hydroxy-3-methoxypropyl)piperidin-4-yl)amino)-2-(3-((5-fluoro-2- |
| | methoxy-4-(methylsulfonyl)phenyl)amino)prop-1-yn-1-yl)benzo[b]thiophene 1,1-dioxide; |
| 1000 | 3-(cyclopropylmethyl)-7-(((3R,4R)-3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1,1-dioxide; |
| 1001 | 3-(cyclopropylmethyl)-7-(((3S,4S)-3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1,1-dioxide; |
| 1002 | 3-(cyclopropylmethyl)-7-((4-(dimethylamino)cyclohexyl)amino)-2-(3-((5-fluoro-2-methoxy-4- |
| | (methylsulfonyl)phenyl)amino)prop-1-yn-1-yl)benzo[b]thiophene 1,1-dioxide; |
| 1003 | 3-(cyclopropylmethyl)-7-((2-(dimethylamino)ethyl)amino)-2-(3-((5-fluoro-2-methoxy-4-(methylsulfonyl) |
| | phenyl)amino)prop-1-yn-1-yl)benzo[b]thiophene 1,1-dioxide; |
| 1004 | 3-(cyclopropylmethyl)-7-(((3S,4R)-3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1,1-dioxide; |
| 1005 | 3-(cyclopropylmethyl)-2-(3-((5-fluoro-2-methoxy-4-(methylsulfonyl)phenyl)amino)prop-1-yn-1-yl)-7-((1- |
| | methylpiperidin-4-yl)amino)benzo[b]thiophene 1,1-dioxide; |
| 1006 | 3-(cyclopropylmethyl)-2-(3-((5-fluoro-2-methoxy-4-(methylsulfonyl)phenyl)amino)prop-1-yn-1-yl)-7- |
| | ((tetrahydro-2H-pyran-4-yl)amino)benzo[b]thiophene 1,1-dioxide; |
| 1007 | 7-((3,3-difluoro-1-methylpiperidin-4-yl)amino)-3-(2,2-difluoroethyl)-2-(3-((5-fluoro-2-methoxy-4- |
| | (methylsulfonyl)phenyl)amino)prop-1-yn-1-yl)benzo[b]thiophene 1,1-dioxide; |
| 1008 | 3-(2,2-difluoroethyl)-7-((1,1-dioxidotetrahydro-2H-thiopyran-4-yl)amino)-2-(3-((5-fluoro-2-methoxy-4- |
| | (methylsulfonyl)phenyl)amino)prop-1-yn-1-yl)benzo[b]thiophene 1,1-dioxide; |
| 1009 | 3-(2,2-difluoroethyl)-7-(((3R,4S)-3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1,1-dioxide; |
| 1010 | 3-(2,2-difluoroethyl)-7-((3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1,1-dioxide; |
| 1011 | 1-(4-((3-(2,2-difluoroethyl)-2-(3-((5-fluoro-2-methoxy-4-(methylsulfonyl)phenyl)amino)prop-1-yn-1-yl)- |
| | 1,1-dioxidobenzo[b]thiophen-7-yl)amino)piperidin-1-yl)-2-(dimethylamino)ethan-1-one; |
| 1012 | 3-(2,2-difluoroethyl)-7-((3-fluoro-1-(2-hydroxy-3-methoxypropyl)piperidin-4-yl)amino)-2-(3-((5-fluoro-2- |
| | methoxy-4-(methylsulfonyl)phenyl)amino)prop-1-yn-1-yl)benzo[b]thiophene 1,1-dioxide; |
| 1013 | 3-(2,2-difluoroethyl)-7-(((3R,4R)-3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1,1-dioxide; |
| 1014 | 3-(2,2-difluoroethyl)-7-(((3S,4S)-3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1,1-dioxide; |
| 1015 | 3-(2,2-difluoroethyl)-7-((4-(dimethylamino)cyclohexyl)amino)-2-(3-((5-fluoro-2-methoxy-4- |
| | (methylsulfonyl)phenyl)amino)prop-1-yn-1-yl)benzo[b]thiophene 1,1-dioxide; |
| 1016 | 3-(2,2-difluoroethyl)-7-((2-(dimethylamino)ethyl)amino)-2-(3-((5-fluoro-2-methoxy-4-(methylsulfonyl) |
| | phenyl)amino)prop-1-yn-1-yl)benzo[b]thiophene 1,1-dioxide; |
| 1017 | 3-(2,2-difluoroethyl)-7-(((3S,4R)-3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1,1-dioxide; |
| 1018 | 3-(2,2-difluoroethyl)-2-(3-((5-fluoro-2-methoxy-4-(methylsulfonyl)phenyl)amino)prop-1-yn-1-yl)-7-((1- |
| | methylpiperidin-4-yl)amino)benzo[b]thiophene 1,1-dioxide; |
| 1019 | 3-(2,2-difluoroethyl)-2-(3-((5-fluoro-2-methoxy-4-(methylsulfonyl)phenyl)amino)prop-1-yn-1-yl)-7- |
| | ((tetrahydro-2H-pyran-4-yl)amino)benzo[b]thiophene 1,1-dioxide; |
| 1020 | 7-((3,3-difluoro-1-methylpiperidin-4-yl)amino)-2-(3-((5-fluoro-2-methoxy-4-(methylsulfonyl)phenyl) |
| | amino)prop-1-yn-1-yl)-3-(1H-pyrrol-1-yl)benzo[b]thiophene 1,1-dioxide; |
| 1021 | 7-((1,1-dioxidotetrahydro-2H-thiopyran-4-yl)amino)-2-(3-((5-fluoro-2-methoxy-4-(methylsulfonyl)phenyl) |
| | amino)prop-1-yn-1-yl)-3-(1H-pyrrol-1-yl)benzo[b]thiophene 1,1-dioxide; |
| 1022 | 7-(((3R,4S)-3-fluoro-1-methylpiperidin-4-yl)amino)-2-(3-((5-fluoro-2-methoxy-4-(methylsulfonyl)phenyl) |
| | amino)prop-1-yn-1-yl)-3-(1H-pyrrol-1-yl)benzo[b]thiophene 1,1-dioxide; |
| 1023 | 7-((3-fluoro-1-methylpiperidin-4-yl)amino)-2-(3-((5-fluoro-2-methoxy-4-(methylsulfonyl)phenyl)amino)prop- |
| | 1-yn-1-yl)-3-(1H-pyrrol-1-yl)benzo[b]thiophene 1,1-dioxide; |
| 1024 | 2-(dimethylamino)-1-(4-((2-(3-((5-fluoro-2-methoxy-4-(methylsulfonyl)phenyl)amino)prop-1-yn-1-yl)-1,1- |
| | dioxido-3-(1H-pyrrol-1-yl)benzo[b]thiophen-7-yl)amino)piperidin-1-yl)ethan-1-one; |
| 1025 | 7-((3-fluoro-1-(2-hydroxy-3-methoxypropyl)piperidin-4-yl)amino)-2-(3-((5-fluoro-2-methoxy-4- |
| | (methylsulfonyl)phenyl)amino)prop-1-yn-1-yl)-3-(1H-pyrrol-1-yl)benzo[b]thiophene 1,1-dioxide; |
| 1026 | 7-(((3R,4R)-3-fluoro-1-methylpiperidin-4-yl)amino)-2-(3-((5-fluoro-2-methoxy-4-(methylsulfonyl)phenyl) |
| | amino)prop-1-yn-1-yl)-3-(1H-pyrrol-1-yl)benzo[b]thiophene 1,1-dioxide; |
| 1027 | 7-(((3S,4S)-3-fluoro-1-methylpiperidin-4-yl)amino)-2-(3-((5-fluoro-2-methoxy-4-(methylsulfonyl)phenyl) |
| | amino)prop-1-yn-1-yl)-3-(1H-pyrrol-1-yl)benzo[b]thiophene 1,1-dioxide; |
| 1028 | 7-((4-(dimethylamino)cyclohexyl)amino)-2-(3-((5-fluoro-2-methoxy-4-(methylsulfonyl)phenyl)amino)prop- |
| | 1-yn-1-yl)-3-(1H-pyrrol-1-yl)benzo[b]thiophene 1,1-dioxide; |
| 1029 | 7-((2-(dimethylamino)ethyl)amino)-2-(3-((5-fluoro-2-methoxy-4-(methylsulfonyl)phenyl)amino)prop-1-yn- |
| | 1-yl)-3-(1H-pyrrol-1-yl)benzo[b]thiophene 1,1-dioxide; |
| 1030 | 7-(((3S,4R)-3-fluoro-1-methylpiperidin-4-yl)amino)-2-(3-((5-fluoro-2-methoxy-4-(methylsulfonyl)phenyl) |
| | amino)prop-1-yn-1-yl)-3-(1H-pyrrol-1-yl)benzo[b]thiophene 1,1-dioxide; |
| 1031 | 2-(3-((5-fluoro-2-methoxy-4-(methylsulfonyl)phenyl)amino)prop-1-yn-1-yl)-7-((1-methylpiperidin-4-yl) |
| | amino)-3-(1H-pyrrol-1-yl)benzo[b]thiophene 1,1-dioxide; |
| 1032 | 2-(3-((5-fluoro-2-methoxy-4-(methylsulfonyl)phenyl)amino)prop-1-yn-1-yl)-3-(1H-pyrrol-1-yl)-7- |
| | ((tetrahydro-2H-pyran-4-yl)amino)benzo[b]thiophene 1,1-dioxide; |
| 1033 | 7-((3,3-difluoro-1-methylpiperidin-4-yl)amino)-3-ethyl-2-(3-((5-fluoro-2-methoxy-4-(methylsulfonyl) |
| | phenyl)amino)prop-1-yn-1-yl)benzo[b]thiophene 1,1-dioxide; |
| 1034 | 7-((1,1-dioxidotetrahydro-2H-thiopyran-4-yl)amino)-3-ethyl-2-(3-((5-fluoro-2-methoxy-4-(methylsulfonyl) |
| | phenyl)amino)prop-1-yn-1-yl)benzo[b]thiophene 1,1-dioxide; |
| 1035 | 3-ethyl-7-(((3R,4S)-3-fluoro-1-methylpiperidin-4-yl)amino)-2-(3-((5-fluoro-2-methoxy-4-(methylsulfonyl) |
| | phenyl)amino)prop-1-yn-1-yl)benzo[b]thiophene 1,1-dioxide; |
| 1036 | 3-ethyl-7-((3-fluoro-1-methylpiperidin-4-yl)amino)-2-(3-((5-fluoro-2-methoxy-4-(methylsulfonyl)phenyl) |
| | amino)prop-1-yn-1-yl)benzo[b]thiophene 1,1-dioxide; |
| 1037 | 2-(dimethylamino)-1-(4-((3-ethyl-2-(3-((5-fluoro-2-methoxy-4-(methylsulfonyl)phenyl)amino)prop-1-yn-1- |
| | yl)-1,1-dioxidobenzo[b]thiophen-7-yl)amino)piperidin-1-yl)ethan-1-one; |
| 1038 | 3-ethyl-7-((3-fluoro-1-(2-hydroxy-3-methoxypropyl)piperidin-4-yl)amino)-2-(3-((5-fluoro-2-methoxy-4- |
| | (methylsulfonyl)phenyl)amino)prop-1-yn-1-yl)benzo[b]thiophene 1,1-dioxide; |
| 1039 | 3-ethyl-7-(((3R,4R)-3-fluoro-1-methylpiperidin-4-yl)amino)-2-(3-((5-fluoro-2-methoxy-4-(methylsulfonyl) |
| | phenyl)amino)prop-1-yn-1-yl)benzo[b]thiophene 1,1-dioxide; |
| 1040 | 3-ethyl-7-(((3S,4S)-3-fluoro-1-methylpiperidin-4-yl)amino)-2-(3-((5-fluoro-2-methoxy-4-(methylsulfonyl) |
| | phenyl)amino)prop-1-yn-1-yl)benzo[b]thiophene 1,1-dioxide; |
| 1041 | 7-((4-(dimethylamino)cyclohexyl)amino)-3-ethyl-2-(3-((5-fluoro-2-methoxy-4-(methylsulfonyl)phenyl) |
| | amino)prop-1-yn-1-yl)benzo[b]thiophene 1,1-dioxide; |
| 1042 | 7-((2-(dimethylamino)ethyl)amino)-3-ethyl-2-(3-((5-fluoro-2-methoxy-4-(methylsulfonyl)phenyl)amino)prop- |
| | 1-yn-1-yl)benzo[b]thiophene 1,1-dioxide; |
| 1043 | 3-ethyl-7-(((3S,4R)-3-fluoro-1-methylpiperidin-4-yl)amino)-2-(3-((5-fluoro-2-methoxy-4-(methylsulfonyl) |
| | phenyl)amino)prop-1-yn-1-yl)benzo[b]thiophene 1,1-dioxide; |
| 1044 | 3-ethyl-2-(3-((5-fluoro-2-methoxy-4-(methylsulfonyl)phenyl)amino)prop-1-yn-1-yl)-7-((1-methylpiperidin- |
| | 4-yl)amino)benzo[b]thiophene 1,1-dioxide; |
| 1045 | 3-ethyl-2-(3-((5-fluoro-2-methoxy-4-(methylsulfonyl)phenyl)amino)prop-1-yn-1-yl)-7-((tetrahydro-2H-pyran- |
| | 4-yl)amino)benzo[b]thiophene 1,1-dioxide; |
| 1046 | 4-((3-(3-(cyanomethyl)-7-((3,3-difluoro-1-methylpiperidin-4-yl)amino)-1,1-dioxidobenzo[b]thiophen-2-yl) |
| | prop-2-yn-1-yl)amino)-3-methoxy-N-methylbenzamide; |
| 1047 | 4-((3-(3-(cyanomethyl)-7-((1,1-dioxidotetrahydro-2H-thiopyran-4-yl)amino)-1,1-dioxidobenzo[b]thiophen- |
| | 2-yl)prop-2-yn-1-yl)amino)-3-methoxy-N-methylbenzamide; |
| 1048 | 4-((3-(3-(cyanomethyl)-7-(((3R,4S)-3-fluoro-1-methylpiperidin-4-yl)amino)-1,1-dioxidobenzo[b]thiophen- |
| | 2-yl)prop-2-yn-1-yl)amino)-3-methoxy-N-methylbenzamide; |
| 1049 | 4-((3-(3-(cyanomethyl)-7-((3-fluoro-1-methylpiperidin-4-yl)amino)-1,1-dioxidobenzo[b]thiophen-2-yl)prop- |
| | 2-yn-1-yl)amino)-3-methoxy-N-methylbenzamide; |
| 1050 | 4-((3-(3-(cyanomethyl)-7-((1-(dimethylglycyl)piperidin-4-yl)amino)-1,1-dioxidobenzo[b]thiophen-2-yl)prop- |
| | 2-yn-1-yl)amino)-3-methoxy-N-methylbenzamide; |
| 1051 | 4-((3-(3-(cyanomethyl)-7-((3-fluoro-1-(2-hydroxy-3-methoxypropyl)piperidin-4-yl)amino)-1,1-dioxidobenzo |
| | [b]thiophen-2-yl)prop-2-yn-1-yl)amino)-3-methoxy-N-methylbenzamide; |
| 1052 | 4-((3-(3-(cyanomethyl)-7-(((3R,4R)-3-fluoro-1-methylpiperidin-4-yl)amino)-1,1-dioxidobenzo[b]thiophen- |
| | 2-yl)prop-2-yn-1-yl)amino)-3-methoxy-N-methylbenzamide; |
| 1053 | 4-((3-(3-(cyanomethyl)-7-(((3S,4S)-3-fluoro-1-methylpiperidin-4-yl)amino)-1,1-dioxidobenzo[b]thiophen- |
| | 2-yl)prop-2-yn-1-yl)amino)-3-methoxy-N-methylbenzamide; |
| 1054 | 4-((3-(3-(cyanomethyl)-7-((4-(dimethylamino)cyclohexyl)amino)-1,1-dioxidobenzo[b]thiophen-2-yl)prop-2- |
| | yn-1-yl)amino)-3-methoxy-N-methylbenzamide; |
| 1055 | 4-((3-(3-(cyanomethyl)-7-((2-(dimethylamino)ethyl)amino)-1,1-dioxidobenzo[b]thiophen-2-yl)prop-2-yn-1- |
| | yl)amino)-3-methoxy-N-methylbenzamide; |
| 1056 | 4-((3-(3-(cyanomethyl)-7-(((3S,4R)-3-fluoro-1-methylpiperidin-4-yl)amino)-1,1-dioxidobenzo[b]thiophen- |
| | 2-yl)prop-2-yn-1-yl)amino)-3-methoxy-N-methylbenzamide; |
| 1057 | 4-((3-(3-(cyanomethyl)-7-((1-methylpiperidin-4-yl)amino)-1,1-dioxidobenzo[b]thiophen-2-yl)prop-2-yn-1- |
| | yl)amino)-3-methoxy-N-methylbenzamide; |
| 1058 | 4-((3-(3-(cyanomethyl)-1,1-dioxido-7-((tetrahydro-2H-pyran-4-yl)amino)benzo[b]thiophen-2-yl)prop-2-yn- |
| | 1-yl)amino)-3-methoxy-N-methylbenzamide; |
| 1059 | 4-((3-(7-((3,3-difluoro-1-methylpiperidin-4-yl)amino)-1,1-dioxido-3-(thiazol-4-yl)benzo[b]thiophen-2-yl)prop- |
| | 2-yn-1-yl)amino)-3-methoxy-N-methylbenzamide; |
| 1060 | 4-((3-(7-((1,1-dioxidotetrahydro-2H-thiopyran-4-yl)amino)-1,1-dioxido-3-(thiazol-4-yl)benzo[b]thiophen-2- |
| | yl)prop-2-yn-1-yl)amino)-3-methoxy-N-methylbenzamide; |
| 1061 | 4-((3-(7-(((3R,4S)-3-fluoro-1-methylpiperidin-4-yl)amino)-1,1-dioxido-3-(thiazol-4-yl)benzo[b]thiophen-2- |
| | yl)prop-2-yn-1-yl)amino)-3-methoxy-N-methylbenzamide; |
| 1062 | 4-((3-(7-((3-fluoro-1-methylpiperidin-4-yl)amino)-1,1-dioxido-3-(thiazol-4-yl)benzo[b]thiophen-2-yl)prop- |
| | 2-yn-1-yl)amino)-3-methoxy-N-methylbenzamide; |
| 1063 | 4-((3-(7-((1-(dimethylglycyl)piperidin-4-yl)amino)-1,1-dioxido-3-(thiazol-4-yl)benzo[b]thiophen-2-yl)prop- |
| | 2-yn-1-yl)amino)-3-methoxy-N-methylbenzamide; |
| 1064 | 4-((3-(7-((3-fluoro-1-(2-hydroxy-3-methoxypropyl)piperidin-4-yl)amino)-1,1-dioxido-3-(thiazol-4-yl)benzo |
| | [b]thiophen-2-yl)prop-2-yn-1-yl)amino)-3-methoxy-N-methylbenzamide; |
| 1065 | 4-((3-(7-(((3R,4R)-3-fluoro-1-methylpiperidin-4-yl)amino)-1,1-dioxido-3-(thiazol-4-yl)benzo[b]thiophen-2- |
| | yl)prop-2-yn-1-yl)amino)-3-methoxy-N-methylbenzamide; |
| 1066 | 4-((3-(7-(((3S,4S)-3-fluoro-1-methylpiperidin-4-yl)amino)-1,1-dioxido-3-(thiazol-4-yl)benzo[b]thiophen-2- |
| | yl)prop-2-yn-1-yl)amino)-3-methoxy-N-methylbenzamide; |
| 1067 | 4-((3-(7-((4-(dimethylamino)cyclohexyl)amino)-1,1-dioxido-3-(thiazol-4-yl)benzo[b]thiophen-2-yl)prop-2- |
| | yn-1-yl)amino)-3-methoxy-N-methylbenzamide; |
| 1068 | 4-((3-(7-((2-(dimethylamino)ethyl)amino)-1,1-dioxido-3-(thiazol-4-yl)benzo[b]thiophen-2-yl)prop-2-yn-1- |
| | yl)amino)-3-methoxy-N-methylbenzamide; |
| 1069 | 4-((3-(7-(((3S,4R)-3-fluoro-1-methylpiperidin-4-yl)amino)-1,1-dioxido-3-(thiazol-4-yl)benzo[b]thiophen-2- |
| | yl)prop-2-yn-1-yl)amino)-3-methoxy-N-methylbenzamide; |
| 1070 | 3-methoxy-N-methyl-4-((3-(7-((1-methylpiperidin-4-yl)amino)-1,1-dioxido-3-(thiazol-4-yl)benzo[b]thiophen- |
| | 2-yl)prop-2-yn-1-yl)amino)benzamide; |
| 1071 | 4-((3-(1,1-dioxido-7-((tetrahydro-2H-pyran-4-yl)amino)-3-(thiazol-4-yl)benzo[b]thiophen-2-yl)prop-2-yn-1- |
| | yl)amino)-3-methoxy-N-methylbenzamide; |
| 1072 | 4-((3-(7-((3,3-difluoro-1-methylpiperidin-4-yl)amino)-1,1-dioxido-3-(2,2,2-trifluoroethyl)benzo[b]thiophen- |
| | 2-yl)prop-2-yn-1-yl)amino)-3-methoxy-N-methylbenzamide; |
| 1073 | 4-((3-(7-((1,1-dioxidotetrahydro-2H-thiopyran-4-yl)amino)-1,1-dioxido-3-(2,2,2-trifluoroethyl)benzo[b]thiophen- |
| | 2-yl)prop-2-yn-1-yl)amino)-3-methoxy-N-methylbenzamide; |
| 1074 | 4-((3-(7-(((3R,4S)-3-fluoro-1-methylpiperidin-4-yl)amino)-1,1-dioxido-3-(2,2,2-trifluoroethyl)benzo[b]thiophen- |
| | 2-yl)prop-2-yn-1-yl)amino)-3-methoxy-N-methylbenzamide; |
| 1075 | 4-((3-(7-((3-fluoro-1-methylpiperidin-4-yl)amino)-1,1-dioxido-3-(2,2,2-trifluoroethyl)benzo[b]thiophen-2- |
| | yl)prop-2-yn-1-yl)amino)-3-methoxy-N-methylbenzamide; |
| 1076 | 4-((3-(7-((1-(dimethylglycyl)piperidin-4-yl)amino)-1,1-dioxido-3-(2,2,2-trifluoroethyl)benzo[b]thiophen-2- |
| | yl)prop-2-yn-1-yl)amino)-3-methoxy-N-methylbenzamide; |
| 1077 | 4-((3-(7-((3-fluoro-1-(2-hydroxy-3-methoxypropyl)piperidin-4-yl)amino)-1,1-dioxido-3-(2,2,2- |
| | trifluoroethyl)benzo[b]thiophen-2-yl)prop-2-yn-1-yl)amino)-3-methoxy-N-methylbenzamide; |
| 1078 | 4-((3-(7-(((3R,4R)-3-fluoro-1-methylpiperidin-4-yl)amino)-1,1-dioxido-3-(2,2,2-trifluoroethyl)benzo[b]thiophen- |
| | 2-yl)prop-2-yn-1-yl)amino)-3-methoxy-N-methylbenzamide; |
| 1079 | 4-((3-(7-(((3S,4S)-3-fluoro-1-methylpiperidin-4-yl)amino)-1,1-dioxido-3-(2,2,2-trifluoroethyl)benzo[b]thiophen- |
| | 2-yl)prop-2-yn-1-yl)amino)-3-methoxy-N-methylbenzamide; |
| 1080 | 4-((3-(7-((4-(dimethylamino)cyclohexyl)amino)-1,1-dioxido-3-(2,2,2-trifluoroethyl)benzo[b]thiophen-2-yl) |
| | prop-2-yn-1-yl)amino)-3-methoxy-N-methylbenzamide; |
| 1081 | 4-((3-(7-((2-(dimethylamino)ethyl)amino)-1,1-dioxido-3-(2,2,2-trifluoroethyl)benzo[b]thiophen-2-yl)prop-2- |
| | yn-1-yl)amino)-3-methoxy-N-methylbenzamide; |
| 1082 | 4-((3-(7-(((3S,4R)-3-fluoro-1-methylpiperidin-4-yl)amino)-1,1-dioxido-3-(2,2,2-trifluoroethyl)benzo[b]thiophen- |
| | 2-yl)prop-2-yn-1-yl)amino)-3-methoxy-N-methylbenzamide; |
| 1083 | 3-methoxy-N-methyl-4-((3-(7-((1-methylpiperidin-4-yl)amino)-1,1-dioxido-3-(2,2,2-trifluoroethyl)benzo[b] |
| | thiophen-2-yl)prop-2-yn-1-yl)amino)benzamide; |
| 1084 | 4-((3-(1,1-dioxido-7-((tetrahydro-2H-pyran-4-yl)amino)-3-(2,2,2-trifluoroethyl)benzo[b]thiophen-2-yl)prop- |
| | 2-yn-1-yl)amino)-3-methoxy-N-methylbenzamide; |
| 1085 | 4-((3-(3-(cyclopropylmethyl)-7-((3,3-difluoro-1-methylpiperidin-4-yl)amino)-1,1-dioxidobenzo[b]thiophen- |
| | 2-yl)prop-2-yn-1-yl)amino)-3-methoxy-N-methylbenzamide; |
| 1086 | 4-((3-(3-(cyclopropylmethyl)-7-((1,1-dioxidotetrahydro-2H-thiopyran-4-yl)amino)-1,1-dioxidobenzo[b]thiophen- |
| | 2-yl)prop-2-yn-1-yl)amino)-3-methoxy-N-methylbenzamide; |
| 1087 | 4-((3-(3-(cyclopropylmethyl)-7-(((3R,4S)-3-fluoro-1-methylpiperidin-4-yl)amino)-1,1-dioxidobenzo[b]thiophen- |
| | 2-yl)prop-2-yn-1-yl)amino)-3-methoxy-N-methylbenzamide; |
| 1088 | 4-((3-(3-(cyclopropylmethyl)-7-((3-fluoro-1-methylpiperidin-4-yl)amino)-1,1-dioxidobenzo[b]thiophen-2- |
| | yl)prop-2-yn-1-yl)amino)-3-methoxy-N-methylbenzamide; |
| 1089 | 4-((3-(3-(cyclopropylmethyl)-7-((1-(dimethylglycyl)piperidin-4-yl)amino)-1,1-dioxidobenzo[b]thiophen-2- |
| | yl)prop-2-yn-1-yl)amino)-3-methoxy-N-methylbenzamide; |
| 1090 | 4-((3-(3-(cyclopropylmethyl)-7-((3-fluoro-1-(2-hydroxy-3-methoxypropyl)piperidin-4-yl)amino)-1,1- |
| | dioxidobenzo[b]thiophen-2-yl)prop-2-yn-1-yl)amino)-3-methoxy-N-methylbenzamide; |
| 1091 | 4-((3-(3-(cyclopropylmethyl)-7-(((3R,4R)-3-fluoro-1-methylpiperidin-4-yl)amino)-1,1-dioxidobenzo[b]thiophen- |
| | 2-yl)prop-2-yn-1-yl)amino)-3-methoxy-N-methylbenzamide; |
| 1092 | 4-((3-(3-(cyclopropylmethyl)-7-(((3S,4S)-3-fluoro-1-methylpiperidin-4-yl)amino)-1,1-dioxidobenzo[b]thiophen- |
| | 2-yl)prop-2-yn-1-yl)amino)-3-methoxy-N-methylbenzamide; |
| 1093 | 4-((3-(3-(cyclopropylmethyl)-7-((4-(dimethylamino)cyclohexyl)amino)-1,1-dioxidobenzo[b]thiophen-2-yl) |
| | prop-2-yn-1-yl)amino)-3-methoxy-N-methylbenzamide; |
| 1094 | 4-((3-(3-(cyclopropylmethyl)-7-((2-(dimethylamino)ethyl)amino)-1,1-dioxidobenzo[b]thiophen-2-yl)prop-2- |
| | yn-1-yl)amino)-3-methoxy-N-methylbenzamide; |
| 1095 | 4-((3-(3-(cyclopropylmethyl)-7-(((3S,4R)-3-fluoro-1-methylpiperidin-4-yl)amino)-1,1-dioxidobenzo[b]thiophen- |
| | 2-yl)prop-2-yn-1-yl)amino)-3-methoxy-N-methylbenzamide; |
| 1096 | 4-((3-(3-(cyclopropylmethyl)-7-((1-methylpiperidin-4-yl)amino)-1,1-dioxidobenzo[b]thiophen-2-yl)prop-2- |
| | yn-1-yl)amino)-3-methoxy-N-methylbenzamide; |
| 1097 | 4-((3-(3-(cyclopropylmethyl)-1,1-dioxido-7-((tetrahydro-2H-pyran-4-yl)amino)benzo[b]thiophen-2-yl)prop- |
| | 2-yn-1-yl)amino)-3-methoxy-N-methylbenzamide; |
| 1098 | 4-((3-(7-((3,3-difluoro-1-methylpiperidin-4-yl)amino)-3-(2,2-difluoroethyl)-1,1-dioxidobenzo[b]thiophen-2- |
| | yl)prop-2-yn-1-yl)amino)-3-methoxy-N-methylbenzamide; |
| 1099 | 4-((3-(3-(2,2-difluoroethyl)-7-((1,1-dioxidotetrahydro-2H-thiopyran-4-yl)amino)-1,1-dioxidobenzo[b]thiophen- |
| | 2-yl)prop-2-yn-1-yl)amino)-3-methoxy-N-methylbenzamide; |
| 1100 | 4-((3-(3-(2,2-difluoroethyl)-7-(((3R,4S)-3-fluoro-1-methylpiperidin-4-yl)amino)-1,1-dioxidobenzo[b]thiophen- |
| | 2-yl)prop-2-yn-1-yl)amino)-3-methoxy-N-methylbenzamide; |
| 1101 | 4-((3-(3-(2,2-difluoroethyl)-7-((3-fluoro-1-methylpiperidin-4-yl)amino)-1,1-dioxidobenzo[b]thiophen-2-yl) |
| | prop-2-yn-1-yl)amino)-3-methoxy-N-methylbenzamide; |
| 1102 | 4-((3-(3-(2,2-difluoroethyl)-7-((1-(dimethylglycyl)piperidin-4-yl)amino)-1,1-dioxidobenzo[b]thiophen-2-yl) |
| | prop-2-yn-1-yl)amino)-3-methoxy-N-methylbenzamide; |
| 1103 | 4-((3-(3-(2,2-difluoroethyl)-7-((3-fluoro-1-(2-hydroxy-3-methoxypropyl)piperidin-4-yl)amino)-1,1- |
| | dioxidobenzo[b]thiophen-2-yl)prop-2-yn-1-yl)amino)-3-methoxy-N-methylbenzamide; |
| 1104 | 4-((3-(3-(2,2-difluoroethyl)-7-(((3R,4R)-3-fluoro-1-methylpiperidin-4-yl)amino)-1,1-dioxidobenzo[b]thiophen- |
| | 2-yl)prop-2-yn-1-yl)amino)-3-methoxy-N-methylbenzamide; |
| 1105 | 4-((3-(3-(2,2-difluoroethyl)-7-(((3S,4S)-3-fluoro-1-methylpiperidin-4-yl)amino)-1,1-dioxidobenzo[b]thiophen- |
| | 2-yl)prop-2-yn-1-yl)amino)-3-methoxy-N-methylbenzamide; |
| 1106 | 4-((3-(3-(2,2-difluoroethyl)-7-((4-(dimethylamino)cyclohexyl)amino)-1,1-dioxidobenzo[b]thiophen-2-yl)prop- |
| | 2-yn-1-yl)amino)-3-methoxy-N-methylbenzamide; |
| 1107 | 4-((3-(3-(2,2-difluoroethyl)-7-((2-(dimethylamino)ethyl)amino)-1,1-dioxidobenzo[b]thiophen-2-yl)prop-2-yn- |
| | 1-yl)amino)-3-methoxy-N-methylbenzamide; |
| 1108 | 4-((3-(3-(2,2-difluoroethyl)-7-(((3S,4R)-3-fluoro-1-methylpiperidin-4-yl)amino)-1,1-dioxidobenzo[b]thiophen- |
| | 2-yl)prop-2-yn-1-yl)amino)-3-methoxy-N-methylbenzamide; |
| 1109 | 4-((3-(3-(2,2-difluoroethyl)-7-((1-methylpiperidin-4-yl)amino)-1,1-dioxidobenzo[b]thiophen-2-yl)prop-2-yn- |
| | 1-yl)amino)-3-methoxy-N-methylbenzamide; |
| 1110 | 4-((3-(3-(2,2-difluoroethyl)-1,1-dioxido-7-((tetrahydro-2H-pyran-4-yl)amino)benzo[b]thiophen-2-yl)prop-2- |
| | yn-1-yl)amino)-3-methoxy-N-methylbenzamide; |
| 1111 | 4-((3-(7-((3,3-difluoro-1-methylpiperidin-4-yl)amino)-1,1-dioxido-3-(1H-pyrrol-1-yl)benzo[b]thiophen-2- |
| | yl)prop-2-yn-1-yl)amino)-3-methoxy-N-methylbenzamide; |
| 1112 | 4-((3-(7-((1,1-dioxidotetrahydro-2H-thiopyran-4-yl)amino)-1,1-dioxido-3-(1H-pyrrol-1-yl)benzo[b]thiophen- |
| | 2-yl)prop-2-yn-1-yl)amino)-3-methoxy-N-methylbenzamide; |
| 1113 | 4-((3-(7-(((3R,4S)-3-fluoro-1-methylpiperidin-4-yl)amino)-1,1-dioxido-3-(1H-pyrrol-1-yl)benzo[b]thiophen- |
| | 2-yl)prop-2-yn-1-yl)amino)-3-methoxy-N-methylbenzamide; |
| 1114 | 4-((3-(7-((3-fluoro-1-methylpiperidin-4-yl)amino)-1,1-dioxido-3-(1H-pyrrol-1-yl)benzo[b]thiophen-2-yl)prop- |
| | 2-yn-1-yl)amino)-3-methoxy-N-methylbenzamide; |
| 1115 | 4-((3-(7-((1-(dimethylglycyl)piperidin-4-yl)amino)-1,1-dioxido-3-(1H-pyrrol-1-yl)benzo[b]thiophen-2-yl)prop- |
| | 2-yn-1-yl)amino)-3-methoxy-N-methylbenzamide; |
| 1116 | 4-((3-(7-((3-fluoro-1-(2-hydroxy-3-methoxypropyl)piperidin-4-yl)amino)-1,1-dioxido-3-(1H-pyrrol-1-yl)benzo |
| | [b]thiophen-2-yl)prop-2-yn-1-yl)amino)-3-methoxy-N-methylbenzamide; |
| 1117 | 4-((3-(7-(((3R,4R)-3-fluoro-1-methylpiperidin-4-yl)amino)-1,1-dioxido-3-(1H-pyrrol-1-yl)benzo[b]thiophen- |
| | 2-yl)prop-2-yn-1-yl)amino)-3-methoxy-N-methylbenzamide; |
| 1118 | 4-((3-(7-(((3S,4S)-3-fluoro-1-methylpiperidin-4-yl)amino)-1,1-dioxido-3-(1H-pyrrol-1-yl)benzo[b]thiophen- |
| | 2-yl)prop-2-yn-1-yl)amino)-3-methoxy-N-methylbenzamide; |
| 1119 | 4-((3-(7-((4-(dimethylamino)cyclohexyl)amino)-1,1-dioxido-3-(1H-pyrrol-1-yl)benzo[b]thiophen-2-yl)prop- |
| | 2-yn-1-yl)amino)-3-methoxy-N-methylbenzamide; |
| 1120 | 4-((3-(7-((2-(dimethylamino)ethyl)amino)-1,1-dioxido-3-(1H-pyrrol-1-yl)benzo[b]thiophen-2-yl)prop-2-yn- |
| | 1-yl)amino)-3-methoxy-N-methylbenzamide; |
| 1121 | 4-((3-(7-(((3S,4R)-3-fluoro-1-methylpiperidin-4-yl)amino)-1,1-dioxido-3-(1H-pyrrol-1-yl)benzo[b]thiophen- |
| | 2-yl)prop-2-yn-1-yl)amino)-3-methoxy-N-methylbenzamide; |
| 1122 | 3-methoxy-N-methyl-4-((3-(7-((1-methylpiperidin-4-yl)amino)-1,1-dioxido-3-(1H-pyrrol-1-yl)benzo[b]thiophen- |
| | 2-yl)prop-2-yn-1-yl)amino)benzamide; |
| 1123 | 4-((3-(1,1-dioxido-3-(1H-pyrrol-1-yl)-7-((tetrahydro-2H-pyran-4-yl)amino)benzo[b]thiophen-2-yl)prop-2-yn- |
| | 1-yl)amino)-3-methoxy-N-methylbenzamide; |
| 1124 | 4-((3-(7-((3,3-difluoro-1-methylpiperidin-4-yl)amino)-3-ethyl-1,1-dioxidobenzo[b]thiophen-2-yl)prop-2-yn- |
| | 1-yl)amino)-3-methoxy-N-methylbenzamide; |
| 1125 | 4-((3-(7-((1,1-dioxidotetrahydro-2H-thiopyran-4-yl)amino)-3-ethyl-1,1-dioxidobenzo[b]thiophen-2-yl)prop- |
| | 2-yn-1-yl)amino)-3-methoxy-N-methylbenzamide; |
| 1126 | 4-((3-(3-ethyl-7-(((3R,4S)-3-fluoro-1-methylpiperidin-4-yl)amino)-1,1-dioxidobenzo[b]thiophen-2-yl)prop- |
| | 2-yn-1-yl)amino)-3-methoxy-N-methylbenzamide; |
| 1127 | 4-((3-(3-ethyl-7-((3-fluoro-1-methylpiperidin-4-yl)amino)-1,1-dioxidobenzo[b]thiophen-2-yl)prop-2-yn-1- |
| | yl)amino)-3-methoxy-N-methylbenzamide; |
| 1128 | 4-((3-(7-((1-(dimethylglycyl)piperidin-4-yl)amino)-3-ethyl-1,1-dioxidobenzo[b]thiophen-2-yl)prop-2-yn-1- |
| | yl)amino)-3-methoxy-N-methylbenzamide; |
| 1129 | 4-((3-(3-ethyl-7-((3-fluoro-1-(2-hydroxy-3-methoxypropyl)piperidin-4-yl)amino)-1,1-dioxidobenzo[b]thiophen- |
| | 2-yl)prop-2-yn-1-yl)amino)-3-methoxy-N-methylbenzamide; |
| 1130 | 4-((3-(3-ethyl-7-(((3R,4R)-3-fluoro-1-methylpiperidin-4-yl)amino)-1,1-dioxidobenzo[b]thiophen-2-yl)prop- |
| | 2-yn-1-yl)amino)-3-methoxy-N-methylbenzamide; |
| 1131 | 4-((3-(3-ethyl-7-(((3S,4S)-3-fluoro-1-methylpiperidin-4-yl)amino)-1,1-dioxidobenzo[b]thiophen-2-yl)prop- |
| | 2-yn-1-yl)amino)-3-methoxy-N-methylbenzamide; |
| 1132 | 4-((3-(7-((4-(dimethylamino)cyclohexyl)amino)-3-ethyl-1,1-dioxidobenzo[b]thiophen-2-yl)prop-2-yn-1-yl) |
| | amino)-3-methoxy-N-methylbenzamide; |
| 1133 | 4-((3-(7-((2-(dimethylamino)ethyl)amino)-3-ethyl-1,1-dioxidobenzo[b]thiophen-2-yl)prop-2-yn-1-yl)amino)- |
| | 3-methoxy-N-methylbenzamide; |
| 1134 | 4-((3-(3-ethyl-7-(((3S,4R)-3-fluoro-1-methylpiperidin-4-yl)amino)-1,1-dioxidobenzo[b]thiophen-2-yl)prop- |
| | 2-yn-1-yl)amino)-3-methoxy-N-methylbenzamide; |
| 1135 | 4-((3-(3-ethyl-7-((1-methylpiperidin-4-yl)amino)-1,1-dioxidobenzo[b]thiophen-2-yl)prop-2-yn-1-yl)amino)- |
| | 3-methoxy-N-methylbenzamide; |
| 1136 | 4-((3-(3-ethyl-1,1-dioxido-7-((tetrahydro-2H-pyran-4-yl)amino)benzo[b]thiophen-2-yl)prop-2-yn-1-yl)amino)- |
| | 3-methoxy-N-methylbenzamide; |
| 1137 | 2-(7-((3,3-difluoro-1-methylpiperidin-4-yl)amino)-2-(3-((2-methoxy-4-(methylsulfonyl)phenyl)amino)prop- |
| | 1-yn-1-yl)-1,1-dioxidobenzo[b]thiophen-3-yl)acetonitrile; |
| 1138 | 2-(7-((1,1-dioxidotetrahydro-2H-thiopyran-4-yl)amino)-2-(3-((2-methoxy-4-(methylsulfonyl)phenyl)amino) |
| | prop-1-yn-1-yl)-1,1-dioxidobenzo[b]thiophen-3-yl)acetonitrile; |
| 1139 | 2-(7-(((3R,4S)-3-fluoro-1-methylpiperidin-4-yl)amino)-2-(3-((2-methoxy-4-(methylsulfonyl)phenyl)amino) |
| | prop-1-yn-1-yl)-1,1-dioxidobenzo[b]thiophen-3-yl)acetonitrile; |
| 1140 | 2-(7-((3-fluoro-1-methylpiperidin-4-yl)amino)-2-(3-((2-methoxy-4-(methylsulfonyl)phenyl)amino)prop-1-yn- |
| | 1-yl)-1,1-dioxidobenzo[b]thiophen-3-yl)acetonitrile; |
| 1141 | 2-(7-((1-(dimethylglycyl)piperidin-4-yl)amino)-2-(3-((2-methoxy-4-(methylsulfonyl)phenyl)amino)prop-1- |
| | yn-1-yl)-1,1-dioxidobenzo[b]thiophen-3-yl)acetonitrile; |
| 1142 | 2-(7-((3-fluoro-1-(2-hydroxy-3-methoxypropyl)piperidin-4-yl)amino)-2-(3-((2-methoxy-4-(methylsulfonyl) |
| | phenyl)amino)prop-1-yn-1-yl)-1,1-dioxidobenzo[b]thiophen-3-yl)acetonitrile; |
| 1143 | 2-(7-(((3R,4R)-3-fluoro-1-methylpiperidin-4-yl)amino)-2-(3-((2-methoxy-4-(methylsulfonyl)phenyl)amino) |
| | prop-1-yn-1-yl)-1,1-dioxidobenzo[b]thiophen-3-yl)acetonitrile; |
| 1144 | 2-(7-(((3S,4S)-3-fluoro-1-methylpiperidin-4-yl)amino)-2-(3-((2-methoxy-4-(methylsulfonyl)phenyl)amino) |
| | prop-1-yn-1-yl)-1,1-dioxidobenzo[b]thiophen-3-yl)acetonitrile; |
| 1145 | 2-(7-((4-(dimethylamino)cyclohexyl)amino)-2-(3-((2-methoxy-4-(methylsulfonyl)phenyl)amino)prop-1-yn- |
| | 1-yl)-1,1-dioxidobenzo[b]thiophen-3-yl)acetonitrile; |
| 1146 | 2-(7-((2-(dimethylamino)ethyl)amino)-2-(3-((2-methoxy-4-(methylsulfonyl)phenyl)amino)prop-1-yn-1-yl)- |
| | 1,1-dioxidobenzo[b]thiophen-3-yl)acetonitrile; |
| 1147 | 2-(7-(((3S,4R)-3-fluoro-1-methylpiperidin-4-yl)amino)-2-(3-((2-methoxy-4-(methylsulfonyl)phenyl)amino) |
| | prop-1-yn-1-yl)-1,1-dioxidobenzo[b]thiophen-3-yl)acetonitrile; |
| 1148 | 2-(2-(3-((2-methoxy-4-(methylsulfonyl)phenyl)amino)prop-1-yn-1-yl)-7-((1-methylpiperidin-4-yl)amino)- |
| | 1,1-dioxidobenzo[b]thiophen-3-yl)acetonitrile; |
| 1149 | 2-(2-(3-((2-methoxy-4-(methylsulfonyl)phenyl)amino)prop-1-yn-1-yl)-1,1-dioxido-7-((tetrahydro-2H-pyran- |
| | 4-yl)amino)benzo[b]thiophen-3-yl)acetonitrile; |
| 1150 | 7-((3,3-difluoro-1-methylpiperidin-4-yl)amino)-2-(3-((2-methoxy-4-(methylsulfonyl)phenyl)amino)prop-1- |
| | yn-1-yl)-3-(thiazol-4-yl)benzo[b]thiophene 1,1-dioxide; |
| 1151 | 7-((1,1-dioxidotetrahydro-2H-thiopyran-4-yl)amino)-2-(3-((2-methoxy-4-(methylsulfonyl)phenyl)amino)prop- |
| | 1-yn-1-yl)-3-(thiazol-4-yl)benzo[b]thiophene 1,1-dioxide; |
| 1152 | 7-(((3R,4S)-3-fluoro-1-methylpiperidin-4-yl)amino)-2-(3-((2-methoxy-4-(methylsulfonyl)phenyl)amino)prop- |
| | 1-yn-1-yl)-3-(thiazol-4-yl)benzo[b]thiophene 1,1-dioxide; |
| 1153 | 7-((3-fluoro-1-methylpiperidin-4-yl)amino)-2-(3-((2-methoxy-4-(methylsulfonyl)phenyl)amino)prop-1-yn-1- |
| | yl)-3-(thiazol-4-yl)benzo[b]thiophene 1,1-dioxide; |
| 1154 | 2-(dimethylamino)-1-(4-((2-(3-((2-methoxy-4-(methylsulfonyl)phenyl)amino)prop-1-yn-1-yl)-1,1-dioxido- |
| | 3-(thiazol-4-yl)benzo[b]thiophen-7-yl)amino)piperidin-1-yl)ethan-1-one; |
| 1155 | 7-((3-fluoro-1-(2-hydroxy-3-methoxypropyl)piperidin-4-yl)amino)-2-(3-((2-methoxy-4-(methylsulfonyl) |
| | phenyl)amino)prop-1-yn-1-yl)-3-(thiazol-4-yl)benzo[b]thiophene 1,1-dioxide; |
| 1156 | 7-(((3R,4R)-3-fluoro-1-methylpiperidin-4-yl)amino)-2-(3-((2-methoxy-4-(methylsulfonyl)phenyl)amino)prop- |
| | 1-yn-1-yl)-3-(thiazol-4-yl)benzo[b]thiophene 1,1-dioxide; |
| 1157 | 7-(((3S,4S)-3-fluoro-1-methylpiperidin-4-yl)amino)-2-(3-((2-methoxy-4-(methylsulfonyl)phenyl)amino)prop- |
| | 1-yn-1-yl)-3-(thiazol-4-yl)benzo[b]thiophene 1,1-dioxide; |
| 1158 | 7-((4-(dimethylamino)cyclohexyl)amino)-2-(3-((2-methoxy-4-(methylsulfonyl)phenyl)amino)prop-1-yn-1- |
| | yl)-3-(thiazol-4-yl)benzo[b]thiophene 1,1-dioxide; |
| 1159 | 7-((2-(dimethylamino)ethyl)amino)-2-(3-((2-methoxy-4-(methylsulfonyl)phenyl)amino)prop-1-yn-1-yl)-3- |
| | (thiazol-4-yl)benzo[b]thiophene 1,1-dioxide; |
| 1160 | 7-(((3S,4R)-3-fluoro-1-methylpiperidin-4-yl)amino)-2-(3-((2-methoxy-4-(methylsulfonyl)phenyl)amino)prop- |
| | 1-yn-1-yl)-3-(thiazol-4-yl)benzo[b]thiophene 1,1-dioxide; |
| 1161 | 2-(3-((2-methoxy-4-(methylsulfonyl)phenyl)amino)prop-1-yn-1-yl)-7-((1-methylpiperidin-4-yl)amino)-3- |
| | (thiazol-4-yl)benzo[b]thiophene 1,1-dioxide; |
| 1162 | 2-(3-((2-methoxy-4-(methylsulfonyl)phenyl)amino)prop-1-yn-1-yl)-7-((tetrahydro-2H-pyran-4-yl)amino)-3- |
| | (thiazol-4-yl)benzo[b]thiophene 1,1-dioxide; |
| 1163 | 7-((3,3-difluoro-1-methylpiperidin-4-yl)amino)-2-(3-((2-methoxy-4-(methylsulfonyl)phenyl)amino)prop-1- |
| | yn-1-yl)-3-(2,2,2-trifluoroethyl)benzo[b]thiophene 1,1-dioxide; |
| 1164 | 7-((1,1-dioxidotetrahydro-2H-thiopyran-4-yl)amino)-2-(3-((2-methoxy-4-(methylsulfonyl)phenyl)amino)prop- |
| | 1-yn-1-yl)-3-(2,2,2-trifluoroethyl)benzo[b]thiophene 1,1-dioxide; |
| 1165 | 7-(((3R,4S)-3-fluoro-1-methylpiperidin-4-yl)amino)-2-(3-((2-methoxy-4-(methylsulfonyl)phenyl)amino)prop- |
| | 1-yn-1-yl)-3-(2,2,2-trifluoroethyl)benzo[b]thiophene 1,1-dioxide; |
| 1166 | 7-((3-fluoro-1-methylpiperidin-4-yl)amino)-2-(3-((2-methoxy-4-(methylsulfonyl)phenyl)amino)prop-1-yn-1- |
| | yl)-3-(2,2,2-trifluoroethyl)benzo[b]thiophene 1,1-dioxide; |
| 1167 | 2-(dimethylamino)-1-(4-((2-(3-((2-methoxy-4-(methylsulfonyl)phenyl)amino)prop-1-yn-1-yl)-1,1-dioxido- |
| | 3-(2,2,2-trifluoroethyl)benzo[b]thiophen-7-yl)amino)piperidin-1-yl)ethan-1-one; |
| 1168 | 7-((3-fluoro-1-(2-hydroxy-3-methoxypropyl)piperidin-4-yl)amino)-2-(3-((2-methoxy-4-(methylsulfonyl) |
| | phenyl)amino)prop-1-yn-1-yl)-3-(2,2,2-trifluoroethyl)benzo[b]thiophene 1,1-dioxide; |
| 1169 | 7-(((3R,4R)-3-fluoro-1-methylpiperidin-4-yl)amino)-2-(3-((2-methoxy-4-(methylsulfonyl)phenyl)amino)prop- |
| | 1-yn-1-yl)-3-(2,2,2-trifluoroethyl)benzo[b]thiophene 1,1-dioxide; |
| 1170 | 7-(((3S,4S)-3-fluoro-1-methylpiperidin-4-yl)amino)-2-(3-((2-methoxy-4-(methylsulfonyl)phenyl)amino)prop- |
| | 1-yn-1-yl)-3-(2,2,2-trifluoroethyl)benzo[b]thiophene 1,1-dioxide; |
| 1171 | 7-((4-(dimethylamino)cyclohexyl)amino)-2-(3-((2-methoxy-4-(methylsulfonyl)phenyl)amino)prop-1-yn-1- |
| | yl)-3-(2,2,2-trifluoroethyl)benzo[b]thiophene 1,1-dioxide; |
| 1172 | 7-((2-(dimethylamino)ethyl)amino)-2-(3-((2-methoxy-4-(methylsulfonyl)phenyl)amino)prop-1-yn-1-yl)-3- |
| | (2,2,2-trifluoroethyl)benzo[b]thiophene 1,1-dioxide; |
| 1173 | 7-(((3S,4R)-3-fluoro-1-methylpiperidin-4-yl)amino)-2-(3-((2-methoxy-4-(methylsulfonyl)phenyl)amino)prop- |
| | 1-yn-1-yl)-3-(2,2,2-trifluoroethyl)benzo[b]thiophene 1,1-dioxide; |
| 1174 | 2-(3-((2-methoxy-4-(methylsulfonyl)phenyl)amino)prop-1-yn-1-yl)-7-((1-methylpiperidin-4-yl)amino)-3- |
| | (2,2,2-trifluoroethyl)benzo[b]thiophene 1,1-dioxide; |
| 1175 | 2-(3-((2-methoxy-4-(methylsulfonyl)phenyl)amino)prop-1-yn-1-yl)-7-((tetrahydro-2H-pyran-4-yl)amino)-3- |
| | (2,2,2-trifluoroethyl)benzo[b]thiophene 1,1-dioxide; |
| 1176 | 3-(cyclopropylmethyl)-7-((3,3-difluoro-1-methylpiperidin-4-yl)amino)-2-(3-((2-methoxy-4- |
| | (methylsulfonyl)phenyl)amino)prop-1-yn-1-yl)benzo[b]thiophene 1,1-dioxide; |
| 1177 | 3-(cyclopropylmethyl)-7-((1,1-dioxidotetrahydro-2H-thiopyran-4-yl)amino)-2-(3-((2-methoxy-4- |
| | (methylsulfonyl)phenyl)amino)prop-1-yn-1-yl)benzo[b]thiophene 1,1-dioxide; |
| 1178 | 3-(cyclopropylmethyl)-7-(((3R,4S)-3-fluoro-1-methylpiperidin-4-yl)amino)-2-(3-((2-methoxy-4- |
| | (methylsulfonyl)phenyl)amino)prop-1-yn-1-yl)benzo[b]thiophene 1,1-dioxide; |
| 1179 | 3-(cyclopropylmethyl)-7-((3-fluoro-1-methylpiperidin-4-yl)amino)-2-(3-((2-methoxy-4-(methylsulfonyl) |
| | phenyl)amino)prop-1-yn-1-yl)benzo[b]thiophene 1,1-dioxide; |
| 1180 | 1-(4-((3-(cyclopropylmethyl)-2-(3-((2-methoxy-4-(methylsulfonyl)phenyl)amino)prop-1-yn-1-yl)-1,1- |
| | dioxidobenzo[b]thiophen-7-yl)amino)piperidin-1-yl)-2-(dimethylamino)ethan-1-one; |
| 1181 | 3-(cyclopropylmethyl)-7-((3-fluoro-1-(2-hydroxy-3-methoxypropyl)piperidin-4-yl)amino)-2-(3-((2-methoxy- |
| | 4-(methylsulfonyl)phenyl)amino)prop-1-yn-1-yl)benzo[b]thiophene 1,1-dioxide; |
| 1182 | 3-(cyclopropylmethyl)-7-(((3R,4R)-3-fluoro-1-methylpiperidin-4-yl)amino)-2-(3-((2-methoxy-4- |
| | (methylsulfonyl)phenyl)amino)prop-1-yn-1-yl)benzo[b]thiophene 1,1-dioxide; |
| 1183 | 3-(cyclopropylmethyl)-7-(((3S,4S)-3-fluoro-1-methylpiperidin-4-yl)amino)-2-(3-((2-methoxy-4- |
| | (methylsulfonyl)phenyl)amino)prop-1-yn-1-yl)benzo[b]thiophene 1,1-dioxide; |
| 1184 | 3-(cyclopropylmethyl)-7-((4-(dimethylamino)cyclohexyl)amino)-2-(3-((2-methoxy-4-(methylsulfonyl) |
| | phenyl)amino)prop-1-yn-1-yl)benzo[b]thiophene 1,1-dioxide; |
| 1185 | 3-(cyclopropylmethyl)-7-((2-(dimethylamino)ethyl)amino)-2-(3-((2-methoxy-4-(methylsulfonyl)phenyl) |
| | amino)prop-1-yn-1-yl)benzo[b]thiophene 1,1-dioxide; |
| 1186 | 3-(cyclopropylmethyl)-7-(((3S,4R)-3-fluoro-1-methylpiperidin-4-yl)amino)-2-(3-((2-methoxy-4- |
| | (methylsulfonyl)phenyl)amino)prop-1-yn-1-yl)benzo[b]thiophene 1,1-dioxide; |
| 1187 | 3-(cyclopropylmethyl)-2-(3-((2-methoxy-4-(methylsulfonyl)phenyl)amino)prop-1-yn-1-yl)-7-((1- |
| | methylpiperidin-4-yl)amino)benzo[b]thiophene 1,1-dioxide; |
| 1188 | 3-(cyclopropylmethyl)-2-(3-((2-methoxy-4-(methylsulfonyl)phenyl)amino)prop-1-yn-1-yl)-7-((tetrahydro- |
| | 2H-pyran-4-yl)amino)benzo[b]thiophene 1,1-dioxide; |
| 1189 | 7-((3,3-difluoro-1-methylpiperidin-4-yl)amino)-3-(2,2-difluoroethyl)-2-(3-((2-methoxy-4-(methylsulfonyl) |
| | phenyl)amino)prop-1-yn-1-yl)benzo[b]thiophene 1,1-dioxide; |
| 1190 | 3-(2,2-difluoroethyl)-7-((1,1-dioxidotetrahydro-2H-thiopyran-4-yl)amino)-2-(3-((2-methoxy-4- |
| | (methylsulfonyl)phenyl)amino)prop-1-yn-1-yl)benzo[b]thiophene 1,1-dioxide; |
| 1191 | 3-(2,2-difluoroethyl)-7-(((3R,4S)-3-fluoro-1-methylpiperidin-4-yl)amino)-2-(3-((2-methoxy-4- |
| | (methylsulfonyl)phenyl)amino)prop-1-yn-1-yl)benzo[b]thiophene 1,1-dioxide; |
| 1192 | 3-(2,2-difluoroethyl)-7-((3-fluoro-1-methylpiperidin-4-yl)amino)-2-(3-((2-methoxy-4-(methylsulfonyl)phenyl) |
| | amino)prop-1-yn-1-yl)benzo[b]thiophene 1,1-dioxide; |
| 1193 | 1-(4-((3-(2,2-difluoroethyl)-2-(3-((2-methoxy-4-(methylsulfonyl)phenyl)amino)prop-1-yn-1-yl)-1,1- |
| | dioxidobenzo[b]thiophen-7-yl)amino)piperidin-1-yl)-2-(dimethylamino)ethan-1-one; |
| 1194 | 3-(2,2-difluoroethyl)-7-((3-fluoro-1-(2-hydroxy-3-methoxypropyl)piperidin-4-yl)amino)-2-(3-((2-methoxy- |
| | 4-(methylsulfonyl)phenyl)amino)prop-1-yn-1-yl)benzo[b]thiophene 1,1-dioxide; |
| 1195 | 3-(2,2-difluoroethyl)-7-(((3R,4R)-3-fluoro-1-methylpiperidin-4-yl)amino)-2-(3-((2-methoxy-4- |
| | (methylsulfonyl)phenyl)amino)prop-1-yn-1-yl)benzo[b]thiophene 1,1-dioxide; |
| 1196 | 3-(2,2-difluoroethyl)-7-(((3S,4S)-3-fluoro-1-methylpiperidin-4-yl)amino)-2-(3-((2-methoxy-4- |
| | (methylsulfonyl)phenyl)amino)prop-1-yn-1-yl)benzo[b]thiophene 1,1-dioxide; |
| 1197 | 3-(2,2-difluoroethyl)-7-((4-(dimethylamino)cyclohexyl)amino)-2-(3-((2-methoxy-4-(methylsulfonyl)phenyl) |
| | amino)prop-1-yn-1-yl)benzo[b]thiophene 1,1-dioxide; |
| 1198 | 3-(2,2-difluoroethyl)-7-((2-(dimethylamino)ethyl)amino)-2-(3-((2-methoxy-4-(methylsulfonyl)phenyl)amino) |
| | prop-1-yn-1-yl)benzo[b]thiophene 1,1-dioxide; |
| 1199 | 3-(2,2-difluoroethyl)-7-(((3S,4R)-3-fluoro-1-methylpiperidin-4-yl)amino)-2-(3-((2-methoxy-4-(methylsulfonyl) |
| | phenyl)amino)prop-1-yn-1-yl)benzo[b]thiophene 1,1-dioxide; |
| 1200 | 3-(2,2-difluoroethyl)-2-(3-((2-methoxy-4-(methylsulfonyl)phenyl)amino)prop-1-yn-1-yl)-7-((1-methylpiperidin- |
| | 4-yl)amino)benzo[b]thiophene 1,1-dioxide; |
| 1201 | 3-(2,2-difluoroethyl)-2-(3-((2-methoxy-4-(methylsulfonyl)phenyl)amino)prop-1-yn-1-yl)-7-((tetrahydro-2H- |
| | pyran-4-yl)amino)benzo[b]thiophene 1,1-dioxide; |
| 1202 | 7-((3,3-difluoro-1-methylpiperidin-4-yl)amino)-2-(3-((2-methoxy-4-(methylsulfonyl)phenyl)amino)prop-1- |
| | yn-1-yl)-3-(1H-pyrrol-1-yl)benzo[b]thiophene 1,1-dioxide; |
| 1203 | 7-((1,1-dioxidotetrahydro-2H-thiopyran-4-yl)amino)-2-(3-((2-methoxy-4-(methylsulfonyl)phenyl)amino)prop- |
| | 1-yn-1-yl)-3-(1H-pyrrol-1-yl)benzo[b]thiophene 1,1-dioxide; |
| 1204 | 7-(((3R,4S)-3-fluoro-1-methylpiperidin-4-yl)amino)-2-(3-((2-methoxy-4-(methylsulfonyl)phenyl)amino)prop- |
| | 1-yn-1-yl)-3-(1H-pyrrol-1-yl)benzo[b]thiophene 1,1-dioxide; |
| 1205 | 7-((3-fluoro-1-methylpiperidin-4-yl)amino)-2-(3-((2-methoxy-4-(methylsulfonyl)phenyl)amino)prop-1-yn-1- |
| | yl)-3-(1H-pyrrol-1-yl)benzo[b]thiophene 1,1-dioxide; |
| 1206 | 2-(dimethylamino)-1-(4-((2-(3-((2-methoxy-4-(methylsulfonyl)phenyl)amino)prop-1-yn-1-yl)-1,1-dioxido- |
| | 3-(1H-pyrrol-1-yl)benzo[b]thiophen-7-yl)amino)piperidin-1-yl)ethan-1-one; |
| 1207 | 7-((3-fluoro-1-(2-hydroxy-3-methoxypropyl)piperidin-4-yl)amino)-2-(3-((2-methoxy-4-(methylsulfonyl)phenyl) |
| | amino)prop-1-yn-1-yl)-3-(1H-pyrrol-1-yl)benzo[b]thiophene 1,1-dioxide; |
| 1208 | 7-(((3R,4R)-3-fluoro-1-methylpiperidin-4-yl)amino)-2-(3-((2-methoxy-4-(methylsulfonyl)phenyl)amino)prop- |
| | 1-yn-1-yl)-3-(1H-pyrrol-1-yl)benzo[b]thiophene 1,1-dioxide; |
| 1209 | 7-(((3S,4S)-3-fluoro-1-methylpiperidin-4-yl)amino)-2-(3-((2-methoxy-4-(methylsulfonyl)phenyl)amino)prop- |
| | 1-yn-1-yl)-3-(1H-pyrrol-1-yl)benzo[b]thiophene 1,1-dioxide; |
| 1210 | 7-((4-(dimethylamino)cyclohexyl)amino)-2-(3-((2-methoxy-4-(methylsulfonyl)phenyl)amino)prop-1-yn-1-yl)- |
| | 3-(1H-pyrrol-1-yl)benzo[b]thiophene 1,1-dioxide; |
| 1211 | 7-((2-(dimethylamino)ethyl)amino)-2-(3-((2-methoxy-4-(methylsulfonyl)phenyl)amino)prop-1-yn-1-yl)-3- |
| | (1H-pyrrol-1-yl)benzo[b]thiophene 1,1-dioxide; |
| 1212 | 7-(((3S,4R)-3-fluoro-1-methylpiperidin-4-yl)amino)-2-(3-((2-methoxy-4-(methylsulfonyl)phenyl)amino)prop- |
| | 1-yn-1-yl)-3-(1H-pyrrol-1-yl)benzo[b]thiophene 1,1-dioxide; |
| 1213 | 2-(3-((2-methoxy-4-(methylsulfonyl)phenyl)amino)prop-1-yn-1-yl)-7-((1-methylpiperidin-4-yl)amino)-3- |
| | (1H-pyrrol-1-yl)benzo[b]thiophene 1,1-dioxide; |
| 1214 | 2-(3-((2-methoxy-4-(methylsulfonyl)phenyl)amino)prop-1-yn-1-yl)-3-(1H-pyrrol-1-yl)-7-((tetrahydro-2H- |
| | pyran-4-yl)amino)benzo[b]thiophene 1,1-dioxide; |
| 1215 | 7-((3,3-difluoro-1-methylpiperidin-4-yl)amino)-3-ethyl-2-(3-((2-methoxy-4-(methylsulfonyl)phenyl)amino) |
| | prop-1-yn-1-yl)benzo[b]thiophene 1,1-dioxide; |
| 1216 | 7-((1,1-dioxidotetrahydro-2H-thiopyran-4-yl)amino)-3-ethyl-2-(3-((2-methoxy-4-(methylsulfonyl)phenyl) |
| | amino)prop-1-yn-1-yl)benzo[b]thiophene 1,1-dioxide; |
| 1217 | 3-ethyl-7-(((3R,4S)-3-fluoro-1-methylpiperidin-4-yl)amino)-2-(3-((2-methoxy-4-(methylsulfonyl)phenyl) |
| | amino)prop-1-yn-1-yl)benzo[b]thiophene 1,1-dioxide; |
| 1218 | 3-ethyl-7-((3-fluoro-1-methylpiperidin-4-yl)amino)-2-(3-((2-methoxy-4-(methylsulfonyl)phenyl)amino)prop- |
| | 1-yn-1-yl)benzo[b]thiophene 1,1-dioxide; |
| 1219 | 2-(dimethylamino)-1-(4-((3-ethyl-2-(3-((2-methoxy-4-(methylsulfonyl)phenyl)amino)prop-1-yn-1-yl)-1,1- |
| | dioxidobenzo[b]thiophen-7-yl)amino)piperidin-1-yl)ethan-1-one; |
| 1220 | 3-ethyl-7-((3-fluoro-1-(2-hydroxy-3-methoxypropyl)piperidin-4-yl)amino)-2-(3-((2-methoxy-4- |
| | (methylsulfonyl)phenyl)amino)prop-1-yn-1-yl)benzo[b]thiophene 1,1-dioxide; |
| 1221 | 3-ethyl-7-(((3R,4R)-3-fluoro-1-methylpiperidin-4-yl)amino)-2-(3-((2-methoxy-4-(methylsulfonyl)phenyl) |
| | amino)prop-1-yn-1-yl)benzo[b]thiophene 1,1-dioxide; |
| 1222 | 3-ethyl-7-(((3S,4S)-3-fluoro-1-methylpiperidin-4-yl)amino)-2-(3-((2-methoxy-4-(methylsulfonyl)phenyl) |
| | amino)prop-1-yn-1-yl)benzo[b]thiophene 1,1-dioxide; |
| 1223 | 7-((4-(dimethylamino)cyclohexyl)amino)-3-ethyl-2-(3-((2-methoxy-4-(methylsulfonyl)phenyl)amino)prop- |
| | 1-yn-1-yl)benzo[b]thiophene 1,1-dioxide; |
| 1224 | 7-((2-(dimethylamino)ethyl)amino)-3-ethyl-2-(3-((2-methoxy-4-(methylsulfonyl)phenyl)amino)prop-1-yn-1- |
| | yl)benzo[b]thiophene 1,1-dioxide; |
| 1225 | 3-ethyl-7-(((3S,4R)-3-fluoro-1-methylpiperidin-4-yl)amino)-2-(3-((2-methoxy-4-(methylsulfonyl)phenyl) |
| | amino)prop-1-yn-1-yl)benzo[b]thiophene 1,1-dioxide; |
| 1226 | 3-ethyl-2-(3-((2-methoxy-4-(methylsulfonyl)phenyl)amino)prop-1-yn-1-yl)-7-((1-methylpiperidin-4-yl) |
| | amino)benzo[b]thiophene 1,1-dioxide; |
| 1227 | 3-ethyl-2-(3-((2-methoxy-4-(methylsulfonyl)phenyl)amino)prop-1-yn-1-yl)-7-((tetrahydro-2H-pyran-4-yl) |
| | amino)benzo[b]thiophene 1,1-dioxide; |
| 1228 | 4-((3-(3-(cyanomethyl)-7-((3,3-difluoro-1-methylpiperidin-4-yl)amino)-1-oxidobenzo[b]thiophen-2-yl)prop- |
| | 2-yn-1-yl)amino)-3-methoxybenzenesulfonamide; |
| 1229 | 4-((3-(3-(cyanomethyl)-7-((1,1-dioxidotetrahydro-2H-thiopyran-4-yl)amino)-1-oxidobenzo[b]thiophen-2-yl) |
| | prop-2-yn-1-yl)amino)-3-methoxybenzenesulfonamide; |
| 1230 | 4-((3-(3-(cyanomethyl)-7-(((3R,4S)-3-fluoro-1-methylpiperidin-4-yl)amino)-1-oxidobenzo[b]thiophen-2-yl) |
| | prop-2-yn-1-yl)amino)-3-methoxybenzenesulfonamide; |
| 1231 | 4-((3-(3-(cyanomethyl)-7-((3-fluoro-1-methylpiperidin-4-yl)amino)-1-oxidobenzo[b]thiophen-2-yl)prop-2- |
| | yn-1-yl)amino)-3-methoxybenzenesulfonamide; |
| 1232 | 4-((3-(3-(cyanomethyl)-7-((1-(dimethylglycyl)piperidin-4-yl)amino)-1-oxidobenzo[b]thiophen-2-yl)prop-2- |
| | yn-1-yl)amino)-3-methoxybenzenesulfonamide; |
| 1233 | 4-((3-(3-(cyanomethyl)-7-((3-fluoro-1-(2-hydroxy-3-methoxypropyl)piperidin-4-yl)amino)-1-oxidobenzo |
| | [b]thiophen-2-yl)prop-2-yn-1-yl)amino)-3-methoxybenzenesulfonamide; |
| 1234 | 4-((3-(3-(cyanomethyl)-7-(((3R,4R)-3-fluoro-1-methylpiperidin-4-yl)amino)-1-oxidobenzo[b]thiophen-2-yl) |
| | prop-2-yn-1-yl)amino)-3-methoxybenzenesulfonamide; |
| 1235 | 4-((3-(3-(cyanomethyl)-7-(((3S,4S)-3-fluoro-1-methylpiperidin-4-yl)amino)-1-oxidobenzo[b]thiophen-2-yl) |
| | prop-2-yn-1-yl)amino)-3-methoxybenzenesulfonamide; |
| 1236 | 4-((3-(3-(cyanomethyl)-7-((4-(dimethylamino)cyclohexyl)amino)-1-oxidobenzo[b]thiophen-2-yl)prop-2-yn- |
| | 1-yl)amino)-3-methoxybenzenesulfonamide; |
| 1237 | 4-((3-(3-(cyanomethyl)-7-((2-(dimethylamino)ethyl)amino)-1-oxidobenzo[b]thiophen-2-yl)prop-2-yn-1-yl) |
| | amino)-3-methoxybenzenesulfonamide; |
| 1238 | 4-((3-(3-(cyanomethyl)-7-(((3S,4R)-3-fluoro-1-methylpiperidin-4-yl)amino)-1-oxidobenzo[b]thiophen-2-yl) |
| | prop-2-yn-1-yl)amino)-3-methoxybenzenesulfonamide; |
| 1239 | 4-((3-(3-(cyanomethyl)-7-((1-methylpiperidin-4-yl)amino)-1-oxidobenzo[b]thiophen-2-yl)prop-2-yn-1-yl) |
| | amino)-3-methoxybenzenesulfonamide; |
| 1240 | 4-((3-(3-(cyanomethyl)-1-oxido-7-((tetrahydro-2H-pyran-4-yl)amino)benzo[b]thiophen-2-yl)prop-2-yn-1-yl) |
| | amino)-3-methoxybenzenesulfonamide; |
| 1241 | 4-((3-(7-((3,3-difluoro-1-methylpiperidin-4-yl)amino)-1-oxido-3-(thiazol-4-yl)benzo[b]thiophen-2-yl)prop- |
| | 2-yn-1-yl)amino)-3-methoxybenzenesulfonamide; |
| 1242 | 4-((3-(7-((1,1-dioxidotetrahydro-2H-thiopyran-4-yl)amino)-1-oxido-3-(thiazol-4-yl)benzo[b]thiophen-2-yl) |
| | prop-2-yn-1-yl)amino)-3-methoxybenzenesulfonamide; |
| 1243 | 4-((3-(7-(((3R,4S)-3-fluoro-1-methylpiperidin-4-yl)amino)-1-oxido-3-(thiazol-4-yl)benzo[b]thiophen-2-yl)prop- |
| | 2-yn-1-yl)amino)-3-methoxybenzenesulfonamide; |
| 1244 | 4-((3-(7-((3-fluoro-1-methylpiperidin-4-yl)amino)-1-oxido-3-(thiazol-4-yl)benzo[b]thiophen-2-yl)prop-2-yn- |
| | 1-yl)amino)-3-methoxybenzenesulfonamide; |
| 1245 | 4-((3-(7-((1-(dimethylglycyl)piperidin-4-yl)amino)-1-oxido-3-(thiazol-4-yl)benzo[b]thiophen-2-yl)prop-2-yn- |
| | 1-yl)amino)-3-methoxybenzenesulfonamide; |
| 1246 | 4-((3-(7-((3-fluoro-1-(2-hydroxy-3-methoxypropyl)piperidin-4-yl)amino)-1-oxido-3-(thiazol-4-yl)benzo[b] |
| | thiophen-2-yl)prop-2-yn-1-yl)amino)-3-methoxybenzenesulfonamide; |
| 1247 | 4-((3-(7-(((3R,4R)-3-fluoro-1-methylpiperidin-4-yl)amino)-1-oxido-3-(thiazol-4-yl)benzo[b]thiophen-2-yl) |
| | prop-2-yn-1-yl)amino)-3-methoxybenzenesulfonamide; |
| 1248 | 4-((3-(7-(((3S,4S)-3-fluoro-1-methylpiperidin-4-yl)amino)-1-oxido-3-(thiazol-4-yl)benzo[b]thiophen-2-yl) |
| | prop-2-yn-1-yl)amino)-3-methoxybenzenesulfonamide; |
| 1249 | 4-((3-(7-((4-(dimethylamino)cyclohexyl)amino)-1-oxido-3-(thiazol-4-yl)benzo[b]thiophen-2-yl)prop-2-yn-1- |
| | yl)amino)-3-methoxybenzenesulfonamide; |
| 1250 | 4-((3-(7-((2-(dimethylamino)ethyl)amino)-1-oxido-3-(thiazol-4-yl)benzo[b]thiophen-2-yl)prop-2-yn-1-yl) |
| | amino)-3-methoxybenzenesulfonamide; |
| 1251 | 4-((3-(7-(((3S,4R)-3-fluoro-1-methylpiperidin-4-yl)amino)-1-oxido-3-(thiazol-4-yl)benzo[b]thiophen-2-yl) |
| | prop-2-yn-1-yl)amino)-3-methoxybenzenesulfonamide; |
| 1252 | 3-methoxy-4-((3-(7-((1-methylpiperidin-4-yl)amino)-1-oxido-3-(thiazol-4-yl)benzo[b]thiophen-2-yl)prop-2- |
| | yn-1-yl)amino)benzenesulfonamide; |
| 1253 | 3-methoxy-4-((3-(1-oxido-7-((tetrahydro-2H-pyran-4-yl)amino)-3-(thiazol-4-yl)benzo[b]thiophen-2-yl)prop- |
| | 2-yn-1-yl)amino)benzenesulfonamide; |
| 1254 | 4-((3-(7-((3,3-difluoro-1-methylpiperidin-4-yl)amino)-1-oxido-3-(2,2,2-trifluoroethyl)benzo[b]thiophen-2- |
| | yl)prop-2-yn-1-yl)amino)-3-methoxybenzenesulfonamide; |
| 1255 | 4-((3-(7-((1,1-dioxidotetrahydro-2H-thiopyran-4-yl)amino)-1-oxido-3-(2,2,2-trifluoroethyl)benzo[b]thiophen- |
| | 2-yl)prop-2-yn-1-yl)amino)-3-methoxybenzenesulfonamide; |
| 1256 | 4-((3-(7-(((3R,4S)-3-fluoro-1-methylpiperidin-4-yl)amino)-1-oxido-3-(2,2,2-trifluoroethyl)benzo[b]thiophen- |
| | 2-yl)prop-2-yn-1-yl)amino)-3-methoxybenzenesulfonamide; |
| 1257 | 4-((3-(7-((3-fluoro-1-methylpiperidin-4-yl)amino)-1-oxido-3-(2,2,2-trifluoroethyl)benzo[b]thiophen-2-yl)prop- |
| | 2-yn-1-yl)amino)-3-methoxybenzenesulfonamide; |
| 1258 | 4-((3-(7-((1-(dimethylglycyl)piperidin-4-yl)amino)-1-oxido-3-(2,2,2-trifluoroethyl)benzo[b]thiophen-2-yl)prop- |
| | 2-yn-1-yl)amino)-3-methoxybenzenesulfonamide; |
| 1259 | 4-((3-(7-((3-fluoro-1-(2-hydroxy-3-methoxypropyl)piperidin-4-yl)amino)-1-oxido-3-(2,2,2-trifluoroethyl)benzo |
| | [b]thiophen-2-yl)prop-2-yn-1-yl)amino)-3-methoxybenzenesulfonamide; |
| 1260 | 4-((3-(7-(((3R,4R)-3-fluoro-1-methylpiperidin-4-yl)amino)-1-oxido-3-(2,2,2-trifluoroethyl)benzo[b]thiophen- |
| | 2-yl)prop-2-yn-1-yl)amino)-3-methoxybenzenesulfonamide; |
| 1261 | 4-((3-(7-(((3S,4S)-3-fluoro-1-methylpiperidin-4-yl)amino)-1-oxido-3-(2,2,2-trifluoroethyl)benzo[b]thiophen- |
| | 2-yl)prop-2-yn-1-yl)amino)-3-methoxybenzenesulfonamide; |
| 1262 | 4-((3-(7-((4-(dimethylamino)cyclohexyl)amino)-1-oxido-3-(2,2,2-trifluoroethyl)benzo[b]thiophen-2-yl)prop- |
| | 2-yn-1-yl)amino)-3-methoxybenzenesulfonamide; |
| 1263 | 4-((3-(7-((2-(dimethylamino)ethyl)amino)-1-oxido-3-(2,2,2-trifluoroethyl)benzo[b]thiophen-2-yl)prop-2-yn- |
| | 1-yl)amino)-3-methoxybenzenesulfonamide; |
| 1264 | 4-((3-(7-(((3S,4R)-3-fluoro-1-methylpiperidin-4-yl)amino)-1-oxido-3-(2,2,2-trifluoroethyl)benzo[b]thiophen- |
| | 2-yl)prop-2-yn-1-yl)amino)-3-methoxybenzenesulfonamide; |
| 1265 | 3-methoxy-4-((3-(7-((1-methylpiperidin-4-yl)amino)-1-oxido-3-(2,2,2-trifluoroethyl)benzo[b]thiophen-2-yl) |
| | prop-2-yn-1-yl)amino)benzenesulfonamide; |
| 1266 | 3-methoxy-4-((3-(1-oxido-7-((tetrahydro-2H-pyran-4-yl)amino)-3-(2,2,2-trifluoroethyl)benzo[b]thiophen-2- |
| | yl)prop-2-yn-1-yl)amino)benzenesulfonamide; |
| 1267 | 4-((3-(3-(cyclopropylmethyl)-7-((3,3-difluoro-1-methylpiperidin-4-yl)amino)-1-oxidobenzo[b]thiophen-2- |
| | yl)prop-2-yn-1-yl)amino)-3-methoxybenzenesulfonamide; |
| 1268 | 4-((3-(3-(cyclopropylmethyl)-7-((1,1-dioxidotetrahydro-2H-thiopyran-4-yl)amino)-1-oxidobenzo[b]thiophen- |
| | 2-yl)prop-2-yn-1-yl)amino)-3-methoxybenzenesulfonamide; |
| 1269 | 4-((3-(3-(cyclopropylmethyl)-7-(((3R,4S)-3-fluoro-1-methylpiperidin-4-yl)amino)-1-oxidobenzo[b]thiophen- |
| | 2-yl)prop-2-yn-1-yl)amino)-3-methoxybenzenesulfonamide; |
| 1270 | 4-((3-(3-(cyclopropylmethyl)-7-((3-fluoro-1-methylpiperidin-4-yl)amino)-1-oxidobenzo[b]thiophen-2-yl)prop- |
| | 2-yn-1-yl)amino)-3-methoxybenzenesulfonamide; |
| 1271 | 4-((3-(3-(cyclopropylmethyl)-7-((1-(dimethylglycyl)piperidin-4-yl)amino)-1-oxidobenzo[b]thiophen-2-yl)prop- |
| | 2-yn-1-yl)amino)-3-methoxybenzenesulfonamide; |
| 1272 | 4-((3-(3-(cyclopropylmethyl)-7-((3-fluoro-1-(2-hydroxy-3-methoxypropyl)piperidin-4-yl)amino)-1-oxidobenzo |
| | [b]thiophen-2-yl)prop-2-yn-1-yl)amino)-3-methoxybenzenesulfonamide; |
| 1273 | 4-((3-(3-(cyclopropylmethyl)-7-(((3R,4R)-3-fluoro-1-methylpiperidin-4-yl)amino)-1-oxidobenzo[b]thiophen- |
| | 2-yl)prop-2-yn-1-yl)amino)-3-methoxybenzenesulfonamide; |
| 1274 | 4-((3-(3-(cyclopropylmethyl)-7-(((3S,4S)-3-fluoro-1-methylpiperidin-4-yl)amino)-1-oxidobenzo[b]thiophen- |
| | 2-yl)prop-2-yn-1-yl)amino)-3-methoxybenzenesulfonamide; |
| 1275 | 4-((3-(3-(cyclopropylmethyl)-7-((4-(dimethylamino)cyclohexyl)amino)-1-oxidobenzo[b]thiophen-2-yl)prop- |
| | 2-yn-1-yl)amino)-3-methoxybenzenesulfonamide; |
| 1276 | 4-((3-(3-(cyclopropylmethyl)-7-((2-(dimethylamino)ethyl)amino)-1-oxidobenzo[b]thiophen-2-yl)prop-2-yn- |
| | 1-yl)amino)-3-methoxybenzenesulfonamide; |
| 1277 | 4-((3-(3-(cyclopropylmethyl)-7-(((3S,4R)-3-fluoro-1-methylpiperidin-4-yl)amino)-1-oxidobenzo[b]thiophen- |
| | 2-yl)prop-2-yn-1-yl)amino)-3-methoxybenzenesulfonamide; |
| 1278 | 4-((3-(3-(cyclopropylmethyl)-7-((1-methylpiperidin-4-yl)amino)-1-oxidobenzo[b]thiophen-2-yl)prop-2-yn- |
| | 1-yl)amino)-3-methoxybenzenesulfonamide; |
| 1279 | 4-((3-(3-(cyclopropylmethyl)-1-oxido-7-((tetrahydro-2H-pyran-4-yl)amino)benzo[b]thiophen-2-yl)prop-2-yn- |
| | 1-yl)amino)-3-methoxybenzenesulfonamide; |
| 1280 | 4-((3-(7-((3,3-difluoro-1-methylpiperidin-4-yl)amino)-3-(2,2-difluoroethyl)-1-oxidobenzo[b]thiophen-2-yl) |
| | prop-2-yn-1-yl)amino)-3-methoxybenzenesulfonamide; |
| 1281 | 4-((3-(3-(2,2-difluoroethyl)-7-((1,1-dioxidotetrahydro-2H-thiopyran-4-yl)amino)-1-oxidobenzo[b]thiophen- |
| | 2-yl)prop-2-yn-1-yl)amino)-3-methoxybenzenesulfonamide; |
| 1282 | 4-((3-(3-(2,2-difluoroethyl)-7-(((3R,4S)-3-fluoro-1-methylpiperidin-4-yl)amino)-1-oxidobenzo[b]thiophen- |
| | 2-yl)prop-2-yn-1-yl)amino)-3-methoxybenzenesulfonamide; |
| 1283 | 4-((3-(3-(2,2-difluoroethyl)-7-((3-fluoro-1-methylpiperidin-4-yl)amino)-1-oxidobenzo[b]thiophen-2-yl)prop- |
| | 2-yn-1-yl)amino)-3-methoxybenzenesulfonamide; |
| 1284 | 4-((3-(3-(2,2-difluoroethyl)-7-((1-(dimethylglycyl)piperidin-4-yl)amino)-1-oxidobenzo[b]thiophen-2-yl)prop- |
| | 2-yn-1-yl)amino)-3-methoxybenzenesulfonamide; |
| 1285 | 4-((3-(3-(2,2-difluoroethyl)-7-((3-fluoro-1-(2-hydroxy-3-methoxypropyl)piperidin-4-yl)amino)-1-oxidobenzo |
| | [b]thiophen-2-yl)prop-2-yn-1-yl)amino)-3-methoxybenzenesulfonamide; |
| 1286 | 4-((3-(3-(2,2-difluoroethyl)-7-(((3R,4R)-3-fluoro-1-methylpiperidin-4-yl)amino)-1-oxidobenzo[b]thiophen- |
| | 2-yl)prop-2-yn-1-yl)amino)-3-methoxybenzenesulfonamide; |
| 1287 | 4-((3-(3-(2,2-difluoroethyl)-7-(((3S,4S)-3-fluoro-1-methylpiperidin-4-yl)amino)-1-oxidobenzo[b]thiophen- |
| | 2-yl)prop-2-yn-1-yl)amino)-3-methoxybenzenesulfonamide; |
| 1288 | 4-((3-(3-(2,2-difluoroethyl)-7-((4-(dimethylamino)cyclohexyl)amino)-1-oxidobenzo[b]thiophen-2-yl)prop-2- |
| | yn-1-yl)amino)-3-methoxybenzenesulfonamide; |
| 1289 | 4-((3-(3-(2,2-difluoroethyl)-7-((2-(dimethylamino)ethyl)amino)-1-oxidobenzo[b]thiophen-2-yl)prop-2-yn-1- |
| | yl)amino)-3-methoxybenzenesulfonamide; |
| 1290 | 4-((3-(3-(2,2-difluoroethyl)-7-(((3S,4R)-3-fluoro-1-methylpiperidin-4-yl)amino)-1-oxidobenzo[b]thiophen- |
| | 2-yl)prop-2-yn-1-yl)amino)-3-methoxybenzenesulfonamide; |
| 1291 | 4-((3-(3-(2,2-difluoroethyl)-7-((1-methylpiperidin-4-yl)amino)-1-oxidobenzo[b]thiophen-2-yl)prop-2-yn-1- |
| | yl)amino)-3-methoxybenzenesulfonamide; |
| 1292 | 4-((3-(3-(2,2-difluoroethyl)-1-oxido-7-((tetrahydro-2H-pyran-4-yl)amino)benzo[b]thiophen-2-yl)prop-2-yn- |
| | 1-yl)amino)-3-methoxybenzenesulfonamide; |
| 1293 | 4-((3-(7-((3,3-difluoro-1-methylpiperidin-4-yl)amino)-1-oxido-3-(1H-pyrrol-1-yl)benzo[b]thiophen-2-yl)prop- |
| | 2-yn-1-yl)amino)-3-methoxybenzenesulfonamide; |
| 1294 | 4-((3-(7-((1,1-dioxidotetrahydro-2H-thiopyran-4-yl)amino)-1-oxido-3-(1H-pyrrol-1-yl)benzo[b]thiophen-2- |
| | yl)prop-2-yn-1-yl)amino)-3-methoxybenzenesulfonamide; |
| 1295 | 4-((3-(7-(((3R,4S)-3-fluoro-1-methylpiperidin-4-yl)amino)-1-oxido-3-(1H-pyrrol-1-yl)benzo[b]thiophen-2- |
| | yl)prop-2-yn-1-yl)amino)-3-methoxybenzenesulfonamide; |
| 1296 | 4-((3-(7-((3-fluoro-1-methylpiperidin-4-yl)amino)-1-oxido-3-(1H-pyrrol-1-yl)benzo[b]thiophen-2-yl)prop-2- |
| | yn-1-yl)amino)-3-methoxybenzenesulfonamide; |
| 1297 | 4-((3-(7-((1-(dimethylglycyl)piperidin-4-yl)amino)-1-oxido-3-(1H-pyrrol-1-yl)benzo[b]thiophen-2-yl)prop- |
| | 2-yn-1-yl)amino)-3-methoxybenzenesulfonamide; |
| 1298 | 4-((3-(7-((3-fluoro-1-(2-hydroxy-3-methoxypropyl)piperidin-4-yl)amino)-1-oxido-3-(1H-pyrrol-1-yl)benzo[b] |
| | thiophen-2-yl)prop-2-yn-1-yl)amino)-3-methoxybenzenesulfonamide; |
| 1299 | 4-((3-(7-(((3R,4R)-3-fluoro-1-methylpiperidin-4-yl)amino)-1-oxido-3-(1H-pyrrol-1-yl)benzo[b]thiophen-2- |
| | yl)prop-2-yn-1-yl)amino)-3-methoxybenzenesulfonamide; |
| 1300 | 4-((3-(7-(((3S,4S)-3-fluoro-1-methylpiperidin-4-yl)amino)-1-oxido-3-(1H-pyrrol-1-yl)benzo[b]thiophen-2- |
| | yl)prop-2-yn-1-yl)amino)-3-methoxybenzenesulfonamide; |
| 1301 | 4-((3-(7-((4-(dimethylamino)cyclohexyl)amino)-1-oxido-3-(1H-pyrrol-1-yl)benzo[b]thiophen-2-yl)prop-2-yn- |
| | 1-yl)amino)-3-methoxybenzenesulfonamide; |
| 1302 | 4-((3-(7-((2-(dimethylamino)ethyl)amino)-1-oxido-3-(1H-pyrrol-1-yl)benzo[b]thiophen-2-yl)prop-2-yn-1-yl) |
| | amino)-3-methoxybenzenesulfonamide; |
| 1303 | 4-((3-(7-(((3S,4R)-3-fluoro-1-methylpiperidin-4-yl)amino)-1-oxido-3-(1H-pyrrol-1-yl)benzo[b]thiophen-2- |
| | yl)prop-2-yn-1-yl)amino)-3-methoxybenzenesulfonamide; |
| 1304 | 3-methoxy-4-((3-(7-((1-methylpiperidin-4-yl)amino)-1-oxido-3-(1H-pyrrol-1-yl)benzo[b]thiophen-2-yl)prop- |
| | 2-yn-1-yl)amino)benzenesulfonamide; |
| 1305 | 3-methoxy-4-((3-(1-oxido-3-(1H-pyrrol-1-yl)-7-((tetrahydro-2H-pyran-4-yl)amino)benzo[b]thiophen-2-yl)prop- |
| | 2-yn-1-yl)amino)benzenesulfonamide; |
| 1306 | 4-((3-(7-((3,3-difluoro-1-methylpiperidin-4-yl)amino)-3-ethyl-1-oxidobenzo[b]thiophen-2-yl)prop-2-yn-1- |
| | yl)amino)-3-methoxybenzenesulfonamide; |
| 1307 | 4-((3-(7-((1,1-dioxidotetrahydro-2H-thiopyran-4-yl)amino)-3-ethyl-1-oxidobenzo[b]thiophen-2-yl)prop-2-yn- |
| | 1-yl)amino)-3-methoxybenzenesulfonamide; |
| 1308 | 4-((3-(3-ethyl-7-(((3R,4S)-3-fluoro-1-methylpiperidin-4-yl)amino)-1-oxidobenzo[b]thiophen-2-yl)prop-2-yn- |
| | 1-yl)amino)-3-methoxybenzenesulfonamide; |
| 1309 | 4-((3-(3-ethyl-7-((3-fluoro-1-methylpiperidin-4-yl)amino)-1-oxidobenzo[b]thiophen-2-yl)prop-2-yn-1-yl) |
| | amino)-3-methoxybenzenesulfonamide; |
| 1310 | 4-((3-(7-((1-(dimethylglycyl)piperidin-4-yl)amino)-3-ethyl-1-oxidobenzo[b]thiophen-2-yl)prop-2-yn-1-yl) |
| | amino)-3-methoxybenzenesulfonamide; |
| 1311 | 4-((3-(3-ethyl-7-((3-fluoro-1-(2-hydroxy-3-methoxypropyl)piperidin-4-yl)amino)-1-oxidobenzo[b]thiophen- |
| | 2-yl)prop-2-yn-1-yl)amino)-3-methoxybenzenesulfonamide; |
| 1312 | 4-((3-(3-ethyl-7-(((3R,4R)-3-fluoro-1-methylpiperidin-4-yl)amino)-1-oxidobenzo[b]thiophen-2-yl)prop-2-yn- |
| | 1-yl)amino)-3-methoxybenzenesulfonamide; |
| 1313 | 4-((3-(3-ethyl-7-(((3S,4S)-3-fluoro-1-methylpiperidin-4-yl)amino)-1-oxidobenzo[b]thiophen-2-yl)prop-2-yn- |
| | 1-yl)amino)-3-methoxybenzenesulfonamide; |
| 1314 | 4-((3-(7-((4-(dimethylamino)cyclohexyl)amino)-3-ethyl-1-oxidobenzo[b]thiophen-2-yl)prop-2-yn-1-yl)amino)- |
| | 3-methoxybenzenesulfonamide; |
| 1315 | 4-((3-(7-((2-(dimethylamino)ethyl)amino)-3-ethyl-1-oxidobenzo[b]thiophen-2-yl)prop-2-yn-1-yl)amino)-3- |
| | methoxybenzenesulfonamide; |
| 1316 | 4-((3-(3-ethyl-7-(((3S,4R)-3-fluoro-1-methylpiperidin-4-yl)amino)-1-oxidobenzo[b]thiophen-2-yl)prop-2-yn- |
| | 1-yl)amino)-3-methoxybenzenesulfonamide; |
| 1317 | 4-((3-(3-ethyl-7-((1-methylpiperidin-4-yl)amino)-1-oxidobenzo[b]thiophen-2-yl)prop-2-yn-1-yl)amino)-3- |
| | methoxybenzenesulfonamide; |
| 1318 | 4-((3-(3-ethyl-1-oxido-7-((tetrahydro-2H-pyran-4-yl)amino)benzo[b]thiophen-2-yl)prop-2-yn-1-yl)amino)- |
| | 3-methoxybenzenesulfonamide; |
| 1319 | 2-(7-((3,3-difluoro-1-methylpiperidin-4-yl)amino)-2-(3-((5-fluoro-2-methoxy-4-(methylsulfonyl)phenyl) |
| | amino)prop-1-yn-1-yl)-1-oxidobenzo[b]thiophen-3-yl)acetonitrile; |
| 1320 | 2-(7-((1,1-dioxidotetrahydro-2H-thiopyran-4-yl)amino)-2-(3-((5-fluoro-2-methoxy-4-(methylsulfonyl)phenyl) |
| | amino)prop-1-yn-1-yl)-1-oxidobenzo[b]thiophen-3-yl)acetonitrile; |
| 1321 | 2-(7-(((3R,4S)-3-fluoro-1-methylpiperidin-4-yl)amino)-2-(3-((5-fluoro-2-methoxy-4-(methylsulfonyl)phenyl) |
| | amino)prop-1-yn-1-yl)-1-oxidobenzo[b]thiophen-3-yl)acetonitrile; |
| 1322 | 2-(7-((3-fluoro-1-methylpiperidin-4-yl)amino)-2-(3-((5-fluoro-2-methoxy-4-(methylsulfonyl)phenyl)amino) |
| | prop-1-yn-1-yl)-1-oxidobenzo[b]thiophen-3-yl)acetonitrile; |
| 1323 | 2-(7-((1-(dimethylglycyl)piperidin-4-yl)amino)-2-(3-((5-fluoro-2-methoxy-4-(methylsulfonyl)phenyl)amino) |
| | prop-1-yn-1-yl)-1-oxidobenzo[b]thiophen-3-yl)acetonitrile; |
| 1324 | 2-(7-((3-fluoro-1-(2-hydroxy-3-methoxypropyl)piperidin-4-yl)amino)-2-(3-((5-fluoro-2-methoxy-4- |
| | (methylsulfonyl)phenyl)amino)prop-1-yn-1-yl)-1-oxidobenzo[b]thiophen-3-yl)acetonitrile; |
| 1325 | 2-(7-(((3R,4R)-3-fluoro-1-methylpiperidin-4-yl)amino)-2-(3-((5-fluoro-2-methoxy-4-(methylsulfonyl)phenyl) |
| | amino)prop-1-yn-1-yl)-1-oxidobenzo[b]thiophen-3-yl)acetonitrile; |
| 1326 | 2-(7-(((3S,4S)-3-fluoro-1-methylpiperidin-4-yl)amino)-2-(3-((5-fluoro-2-methoxy-4-(methylsulfonyl)phenyl) |
| | amino)prop-1-yn-1-yl)-1-oxidobenzo[b]thiophen-3-yl)acetonitrile; |
| 1327 | 2-(7-((4-(dimethylamino)cyclohexyl)amino)-2-(3-((5-fluoro-2-methoxy-4-(methylsulfonyl)phenyl)amino)prop- |
| | 1-yn-1-yl)-1-oxidobenzo[b]thiophen-3-yl)acetonitrile; |
| 1328 | 2-(7-((2-(dimethylamino)ethyl)amino)-2-(3-((5-fluoro-2-methoxy-4-(methylsulfonyl)phenyl)amino)prop-1- |
| | yn-1-yl)-1-oxidobenzo[b]thiophen-3-yl)acetonitrile; |
| 1329 | 2-(7-(((3S,4R)-3-fluoro-1-methylpiperidin-4-yl)amino)-2-(3-((5-fluoro-2-methoxy-4-(methylsulfonyl)phenyl) |
| | amino)prop-1-yn-1-yl)-1-oxidobenzo[b]thiophen-3-yl)acetonitrile; |
| 1330 | 2-(2-(3-((5-fluoro-2-methoxy-4-(methylsulfonyl)phenyl)amino)prop-1-yn-1-yl)-7-((1-methylpiperidin-4-yl) |
| | amino)-1-oxidobenzo[b]thiophen-3-yl)acetonitrile; |
| 1331 | 2-(2-(3-((5-fluoro-2-methoxy-4-(methylsulfonyl)phenyl)amino)prop-1-yn-1-yl)-1-oxido-7-((tetrahydro-2H- |
| | pyran-4-yl)amino)benzo[b]thiophen-3-yl)acetonitrile; |
| 1332 | 7-((3,3-difluoro-1-methylpiperidin-4-yl)amino)-2-(3-((5-fluoro-2-methoxy-4-(methylsulfonyl)phenyl)amino) |
| | prop-1-yn-1-yl)-3-(thiazol-4-yl)benzo[b]thiophene 1-oxide; |
| 1333 | 4-((2-(3-((5-fluoro-2-methoxy-4-(methylsulfonyl)phenyl)amino)prop-1-yn-1-yl)-1-oxido-3-(thiazol-4-yl)benzo |
| | [b]thiophen-7-yl)amino)tetrahydro-2H-thiopyran 1,1-dioxide; |
| 1334 | 7-(((3R,4S)-3-fluoro-1-methylpiperidin-4-yl)amino)-2-(3-((5-fluoro-2-methoxy-4-(methylsulfonyl)phenyl)amino) |
| | prop-1-yn-1-yl)-3-(thiazol-4-yl)benzo[b]thiophene 1-oxide; |
| 1335 | 7-((3-fluoro-1-methylpiperidin-4-yl)amino)-2-(3-((5-fluoro-2-methoxy-4-(methylsulfonyl)phenyl)amino)prop- |
| | 1-yn-1-yl)-3-(thiazol-4-yl)benzo[b]thiophene 1-oxide; |
| 1336 | 2-(dimethylamino)-1-(4-((2-(3-((5-fluoro-2-methoxy-4-(methylsulfonyl)phenyl)amino)prop-1-yn-1-yl)-1-oxido- |
| | 3-(thiazol-4-yl)benzo[b]thiophen-7-yl)amino)piperidin-1-yl)ethan-1-one; |
| 1337 | 7-((3-fluoro-1-(2-hydroxy-3-methoxypropyl)piperidin-4-yl)amino)-2-(3-((5-fluoro-2-methoxy-4-(methylsulfonyl) |
| | phenyl)amino)prop-1-yn-1-yl)-3-(thiazol-4-yl)benzo[b]thiophene 1-oxide; |
| 1338 | 7-(((3R,4R)-3-fluoro-1-methylpiperidin-4-yl)amino)-2-(3-((5-fluoro-2-methoxy-4-(methylsulfonyl)phenyl)amino) |
| | prop-1-yn-1-yl)-3-(thiazol-4-yl)benzo[b]thiophene 1-oxide; |
| 1339 | 7-(((3S,4S)-3-fluoro-1-methylpiperidin-4-yl)amino)-2-(3-((5-fluoro-2-methoxy-4-(methylsulfonyl)phenyl)amino) |
| | prop-1-yn-1-yl)-3-(thiazol-4-yl)benzo[b]thiophene 1-oxide; |
| 1340 | 7-((4-(dimethylamino)cyclohexyl)amino)-2-(3-((5-fluoro-2-methoxy-4-(methylsulfonyl)phenyl)amino)prop- |
| | 1-yn-1-yl)-3-(thiazol-4-yl)benzo[b]thiophene 1-oxide; |
| 1341 | 7-((2-(dimethylamino)ethyl)amino)-2-(3-((5-fluoro-2-methoxy-4-(methylsulfonyl)phenyl)amino)prop-1-yn- |
| | 1-yl)-3-(thiazol-4-yl)benzo[b]thiophene 1-oxide; |
| 1342 | 7-(((3S,4R)-3-fluoro-1-methylpiperidin-4-yl)amino)-2-(3-((5-fluoro-2-methoxy-4-(methylsulfonyl)phenyl)amino) |
| | prop-1-yn-1-yl)-3-(thiazol-4-yl)benzo[b]thiophene 1-oxide; |
| 1343 | 2-(3-((5-fluoro-2-methoxy-4-(methylsulfonyl)phenyl)amino)prop-1-yn-1-yl)-7-((1-methylpiperidin-4-yl)amino)- |
| | 3-(thiazol-4-yl)benzo[b]thiophene 1-oxide; |
| 1344 | 2-(3-((5-fluoro-2-methoxy-4-(methylsulfonyl)phenyl)amino)prop-1-yn-1-yl)-7-((tetrahydro-2H-pyran-4-yl) |
| | amino)-3-(thiazol-4-yl)benzo[b]thiophene 1-oxide; |
| 1345 | 7-((3,3-difluoro-1-methylpiperidin-4-yl)amino)-2-(3-((5-fluoro-2-methoxy-4-(methylsulfonyl)phenyl)amino) |
| | prop-1-yn-1-yl)-3-(2,2,2-trifluoroethyl)benzo[b]thiophene 1-oxide; |
| 1346 | 4-((2-(3-((5-fluoro-2-methoxy-4-(methylsulfonyl)phenyl)amino)prop-1-yn-1-yl)-1-oxido-3-(2,2,2-trifluoroethyl) |
| | benzo[b]thiophen-7-yl)amino)tetrahydro-2H-thiopyran 1,1-dioxide; |
| 1347 | 7-(((3R,4S)-3-fluoro-1-methylpiperidin-4-yl)amino)-2-(3-((5-fluoro-2-methoxy-4-(methylsulfonyl)phenyl) |
| | amino)prop-1-yn-1-yl)-3-(2,2,2-trifluoroethyl)benzo[b]thiophene 1-oxide; |
| 1348 | 7-((3-fluoro-1-methylpiperidin-4-yl)amino)-2-(3-((5-fluoro-2-methoxy-4-(methylsulfonyl)phenyl)amino)prop- |
| | 1-yn-1-yl)-3-(2,2,2-trifluoroethyl)benzo[b]thiophene 1-oxide; |
| 1349 | 2-(dimethylamino)-1-(4-((2-(3-((5-fluoro-2-methoxy-4-(methylsulfonyl)phenyl)amino)prop-1-yn-1-yl)-1-oxido- |
| | 3-(2,2,2-trifluoroethyl)benzo[b]thiophen-7-yl)amino)piperidin-1-yl)ethan-1-one; |
| 1350 | 7-((3-fluoro-1-(2-hydroxy-3-methoxypropyl)piperidin-4-yl)amino)-2-(3-((5-fluoro-2-methoxy-4-(methylsulfonyl) |
| | phenyl)amino)prop-1-yn-1-yl)-3-(2,2,2-trifluoroethyl)benzo[b]thiophene 1-oxide; |
| 1351 | 7-(((3R,4R)-3-fluoro-1-methylpiperidin-4-yl)amino)-2-(3-((5-fluoro-2-methoxy-4-(methylsulfonyl)phenyl) |
| | amino)prop-1-yn-1-yl)-3-(2,2,2-trifluoroethyl)benzo[b]thiophene 1-oxide; |
| 1352 | 7-(((3S,4S)-3-fluoro-1-methylpiperidin-4-yl)amino)-2-(3-((5-fluoro-2-methoxy-4-(methylsulfonyl)phenyl) |
| | amino)prop-1-yn-1-yl)-3-(2,2,2-trifluoroethyl)benzo[b]thiophene 1-oxide; |
| 1353 | 7-((4-(dimethylamino)cyclohexyl)amino)-2-(3-((5-fluoro-2-methoxy-4-(methylsulfonyl)phenyl)amino)prop- |
| | 1-yn-1-yl)-3-(2,2,2-trifluoroethyl)benzo[b]thiophene 1-oxide; |
| 1354 | 7-((2-(dimethylamino)ethyl)amino)-2-(3-((5-fluoro-2-methoxy-4-(methylsulfonyl)phenyl)amino)prop-1-yn- |
| | 1-yl)-3-(2,2,2-trifluoroethyl)benzo[b]thiophene 1-oxide; |
| 1355 | 7-(((3S,4R)-3-fluoro-1-methylpiperidin-4-yl)amino)-2-(3-((5-fluoro-2-methoxy-4-(methylsulfonyl)phenyl) |
| | amino)prop-1-yn-1-yl)-3-(2,2,2-trifluoroethyl)benzo[b]thiophene 1-oxide; |
| 1356 | 2-(3-((5-fluoro-2-methoxy-4-(methylsulfonyl)phenyl)amino)prop-1-yn-1-yl)-7-((1-methylpiperidin-4-yl) |
| | amino)-3-(2,2,2-trifluoroethyl)benzo[b]thiophene 1-oxide; |
| 1357 | 2-(3-((5-fluoro-2-methoxy-4-(methylsulfonyl)phenyl)amino)prop-1-yn-1-yl)-7-((tetrahydro-2H-pyran-4-yl) |
| | amino)-3-(2,2,2-trifluoroethyl)benzo[b]thiophene 1-oxide; |
| 1358 | 3-(cyclopropylmethyl)-7-((3,3-difluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1-oxide; |
| 1359 | 4-((3-(cyclopropylmethyl)-2-(3-((5-fluoro-2-methoxy-4-(methylsulfonyl)phenyl)amino)prop-1-yn-1-yl)-1- |
| | oxidobenzo[b]thiophen-7-yl)amino)tetrahydro-2H-thiopyran 1,1-dioxide; |
| 1360 | 3-(cyclopropylmethyl)-7-(((3R,4S)-3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1-oxide; |
| 1361 | 3-(cyclopropylmethyl)-7-((3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1-oxide; |
| 1362 | 1-(4-((3-(cyclopropylmethyl)-2-(3-((5-fluoro-2-methoxy-4-(methylsulfonyl)phenyl)amino)prop-1-yn-1-yl)- |
| | 1-oxidobenzo[b]thiophen-7-yl)amino)piperidin-1-yl)-2-(dimethylamino)ethan-1-one; |
| 1363 | 3-(cyclopropylmethyl)-7-((3-fluoro-1-(2-hydroxy-3-methoxypropyl)piperidin-4-yl)amino)-2-(3-((5-fluoro-2- |
| | methoxy-4-(methylsulfonyl)phenyl)amino)prop-1-yn-1-yl)benzo[b]thiophene 1-oxide; |
| 1364 | 3-(cyclopropylmethyl)-7-(((3R,4R)-3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1-oxide; |
| 1365 | 3-(cyclopropylmethyl)-7-(((3S,4S)-3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1-oxide; |
| 1366 | 3-(cyclopropylmethyl)-7-((4-(dimethylamino)cyclohexyl)amino)-2-(3-((5-fluoro-2-methoxy-4-(methylsulfonyl) |
| | phenyl)amino)prop-1-yn-1-yl)benzo[b]thiophene 1-oxide; |
| 1367 | 3-(cyclopropylmethyl)-7-((2-(dimethylamino)ethyl)amino)-2-(3-((5-fluoro-2-methoxy-4-(methylsulfonyl) |
| | phenyl)amino)prop-1-yn-1-yl)benzo[b]thiophene 1-oxide; |
| 1368 | 3-(cyclopropylmethyl)-7-(((3S,4R)-3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1-oxide; |
| 1369 | 3-(cyclopropylmethyl)-2-(3-((5-fluoro-2-methoxy-4-(methylsulfonyl)phenyl)amino)prop-1-yn-1-yl)-7-((1- |
| | methylpiperidin-4-yl)amino)benzo[b]thiophene 1-oxide; |
| 1370 | 3-(cyclopropylmethyl)-2-(3-((5-fluoro-2-methoxy-4-(methylsulfonyl)phenyl)amino)prop-1-yn-1-yl)-7- |
| | ((tetrahydro-2H-pyran-4-yl)amino)benzo[b]thiophene 1-oxide; |
| 1371 | 7-((3,3-difluoro-1-methylpiperidin-4-yl)amino)-3-(2,2-difluoroethyl)-2-(3-((5-fluoro-2-methoxy-4- |
| | (methylsulfonyl)phenyl)amino)prop-1-yn-1-yl)benzo[b]thiophene 1-oxide; |
| 1372 | 4-((3-(2,2-difluoroethyl)-2-(3-((5-fluoro-2-methoxy-4-(methylsulfonyl)phenyl)amino)prop-1-yn-1-yl)-1- |
| | oxidobenzo[b]thiophen-7-yl)amino)tetrahydro-2H-thiopyran 1,1-dioxide; |
| 1373 | 3-(2,2-difluoroethyl)-7-(((3R,4S)-3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1-oxide; |
| 1374 | 3-(2,2-difluoroethyl)-7-((3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1-oxide; |
| 1375 | 1-(4-((3-(2,2-difluoroethyl)-2-(3-((5-fluoro-2-methoxy-4-(methylsulfonyl)phenyl)amino)prop-1-yn-1-yl)-1- |
| | oxidobenzo[b]thiophen-7-yl)amino)piperidin-1-yl)-2-(dimethylamino)ethan-1-one; |
| 1376 | 3-(2,2-difluoroethyl)-7-((3-fluoro-1-(2-hydroxy-3-methoxypropyl)piperidin-4-yl)amino)-2-(3-((5-fluoro-2- |
| | methoxy-4-(methylsulfonyl)phenyl)amino)prop-1-yn-1-yl)benzo[b]thiophene 1-oxide; |
| 1377 | 3-(2,2-difluoroethyl)-7-(((3R,4R)-3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1-oxide; |
| 1378 | 3-(2,2-difluoroethyl)-7-(((3S,4S)-3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1-oxide; |
| 1379 | 3-(2,2-difluoroethyl)-7-((4-(dimethylamino)cyclohexyl)amino)-2-(3-((5-fluoro-2-methoxy-4- |
| | (methylsulfonyl)phenyl)amino)prop-1-yn-1-yl)benzo[b]thiophene 1-oxide; |
| 1380 | 3-(2,2-difluoroethyl)-7-((2-(dimethylamino)ethyl)amino)-2-(3-((5-fluoro-2-methoxy-4-(methylsulfonyl) |
| | phenyl)amino)prop-1-yn-1-yl)benzo[b]thiophene 1-oxide; |
| 1381 | 3-(2,2-difluoroethyl)-7-(((3S,4R)-3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1-oxide; |
| 1382 | 3-(2,2-difluoroethyl)-2-(3-((5-fluoro-2-methoxy-4-(methylsulfonyl)phenyl)amino)prop-1-yn-1-yl)-7-((1- |
| | methylpiperidin-4-yl)amino)benzo[b]thiophene 1-oxide; |
| 1383 | 3-(2,2-difluoroethyl)-2-(3-((5-fluoro-2-methoxy-4-(methylsulfonyl)phenyl)amino)prop-1-yn-1-yl)-7- |
| | ((tetrahydro-2H-pyran-4-yl)amino)benzo[b]thiophene 1-oxide; |
| 1384 | 7-((3,3-difluoro-1-methylpiperidin-4-yl)amino)-2-(3-((5-fluoro-2-methoxy-4-(methylsulfonyl)phenyl)amino) |
| | prop-1-yn-1-yl)-3-(1H-pyrrol-1-yl)benzo[b]thiophene 1-oxide; |
| 1385 | 4-((2-(3-((5-fluoro-2-methoxy-4-(methylsulfonyl)phenyl)amino)prop-1-yn-1-yl)-1-oxido-3-(1H-pyrrol-1-yl) |
| | benzo[b]thiophen-7-yl)amino)tetrahydro-2H-thiopyran 1,1-dioxide; |
| 1386 | 7-(((3R,4S)-3-fluoro-1-methylpiperidin-4-yl)amino)-2-(3-((5-fluoro-2-methoxy-4-(methylsulfonyl)phenyl) |
| | amino)prop-1-yn-1-yl)-3-(1H-pyrrol-1-yl)benzo[b]thiophene 1-oxide; |
| 1387 | 7-((3-fluoro-1-methylpiperidin-4-yl)amino)-2-(3-((5-fluoro-2-methoxy-4-(methylsulfonyl)phenyl)amino) |
| | prop-1-yn-1-yl)-3-(1H-pyrrol-1-yl)benzo[b]thiophene 1-oxide; |
| 1388 | 2-(dimethylamino)-1-(4-((2-(3-((5-fluoro-2-methoxy-4-(methylsulfonyl)phenyl)amino)prop-1-yn-1-yl)-1- |
| | oxido-3-(1H-pyrrol-1-yl)benzo[b]thiophen-7-yl)amino)piperidin-1-yl)ethan-1-one; |
| 1389 | 7-((3-fluoro-1-(2-hydroxy-3-methoxypropyl)piperidin-4-yl)amino)-2-(3-((5-fluoro-2-methoxy-4- |
| | (methylsulfonyl)phenyl)amino)prop-1-yn-1-yl)-3-(1H-pyrrol-1-yl)benzo[b]thiophene 1-oxide; |
| 1390 | 7-(((3R,4R)-3-fluoro-1-methylpiperidin-4-yl)amino)-2-(3-((5-fluoro-2-methoxy-4-(methylsulfonyl)phenyl) |
| | amino)prop-1-yn-1-yl)-3-(1H-pyrrol-1-yl)benzo[b]thiophene 1-oxide; |
| 1391 | 7-(((3S,4S)-3-fluoro-1-methylpiperidin-4-yl)amino)-2-(3-((5-fluoro-2-methoxy-4-(methylsulfonyl)phenyl) |
| | amino)prop-1-yn-1-yl)-3-(1H-pyrrol-1-yl)benzo[b]thiophene 1-oxide; |
| 1392 | 7-((4-(dimethylamino)cyclohexyl)amino)-2-(3-((5-fluoro-2-methoxy-4-(methylsulfonyl)phenyl)amino)prop- |
| | 1-yn-1-yl)-3-(1H-pyrrol-1-yl)benzo[b]thiophene 1-oxide; |
| 1393 | 7-((2-(dimethylamino)ethyl)amino)-2-(3-((5-fluoro-2-methoxy-4-(methylsulfonyl)phenyl)amino)prop-1-yn- |
| | 1-yl)-3-(1H-pyrrol-1-yl)benzo[b]thiophene 1-oxide; |
| 1394 | 7-(((3S,4R)-3-fluoro-1-methylpiperidin-4-yl)amino)-2-(3-((5-fluoro-2-methoxy-4-(methylsulfonyl)phenyl) |
| | amino)prop-1-yn-1-yl)-3-(1H-pyrrol-1-yl)benzo[b]thiophene 1-oxide; |
| 1395 | 2-(3-((5-fluoro-2-methoxy-4-(methylsulfonyl)phenyl)amino)prop-1-yn-1-yl)-7-((1-methylpiperidin-4-yl) |
| | amino)-3-(1H-pyrrol-1-yl)benzo[b]thiophene 1-oxide; |
| 1396 | 2-(3-((5-fluoro-2-methoxy-4-(methylsulfonyl)phenyl)amino)prop-1-yn-1-yl)-3-(1H-pyrrol-1-yl)-7- |
| | ((tetrahydro-2H-pyran-4-yl)amino)benzo[b]thiophene 1-oxide; |
| 1397 | 7-((3,3-difluoro-1-methylpiperidin-4-yl)amino)-3-ethyl-2-(3-((5-fluoro-2-methoxy-4-(methylsulfonyl) |
| | phenyl)amino)prop-1-yn-1-yl)benzo[b]thiophene 1-oxide; |
| 1398 | 4-((3-ethyl-2-(3-((5-fluoro-2-methoxy-4-(methylsulfonyl)phenyl)amino)prop-1-yn-1-yl)-1-oxidobenzo[b]thiophen- |
| | 7-yl)amino)tetrahydro-2H-thiopyran 1,1-dioxide; |
| 1399 | 3-ethyl-7-(((3R,4S)-3-fluoro-1-methylpiperidin-4-yl)amino)-2-(3-((5-fluoro-2-methoxy-4-(methylsulfonyl) |
| | phenyl)amino)prop-1-yn-1-yl)benzo[b]thiophene 1-oxide; |
| 1400 | 3-ethyl-7-((3-fluoro-1-methylpiperidin-4-yl)amino)-2-(3-((5-fluoro-2-methoxy-4-(methylsulfonyl)phenyl) |
| | amino)prop-1-yn-1-yl)benzo[b]thiophene 1-oxide; |
| 1401 | 2-(dimethylamino)-1-(4-((3-ethyl-2-(3-((5-fluoro-2-methoxy-4-(methylsulfonyl)phenyl)amino)prop-1-yn-1- |
| | yl)-1-oxidobenzo[b]thiophen-7-yl)amino)piperidin-1-yl)ethan-1-one; |
| 1402 | 3-ethyl-7-((3-fluoro-1-(2-hydroxy-3-methoxypropyl)piperidin-4-yl)amino)-2-(3-((5-fluoro-2-methoxy-4- |
| | (methylsulfonyl)phenyl)amino)prop-1-yn-1-yl)benzo[b]thiophene 1-oxide; |
| 1403 | 3-ethyl-7-(((3R,4R)-3-fluoro-1-methylpiperidin-4-yl)amino)-2-(3-((5-fluoro-2-methoxy-4-(methylsulfonyl) |
| | phenyl)amino)prop-1-yn-1-yl)benzo[b]thiophene 1-oxide; |
| 1404 | 3-ethyl-7-(((3S,4S)-3-fluoro-1-methylpiperidin-4-yl)amino)-2-(3-((5-fluoro-2-methoxy-4-(methylsulfonyl) |
| | phenyl)amino)prop-1-yn-1-yl)benzo[b]thiophene 1-oxide; |
| 1405 | 7-((4-(dimethylamino)cyclohexyl)amino)-3-ethyl-2-(3-((5-fluoro-2-methoxy-4-(methylsulfonyl)phenyl)amino) |
| | prop-1-yn-1-yl)benzo[b]thiophene 1-oxide; |
| 1406 | 7-((2-(dimethylamino)ethyl)amino)-3-ethyl-2-(3-((5-fluoro-2-methoxy-4-(methylsulfonyl)phenyl)amino)prop- |
| | 1-yn-1-yl)benzo[b]thiophene 1-oxide; |
| 1407 | 3-ethyl-7-(((3S,4R)-3-fluoro-1-methylpiperidin-4-yl)amino)-2-(3-((5-fluoro-2-methoxy-4-(methylsulfonyl) |
| | phenyl)amino)prop-1-yn-1-yl)benzo[b]thiophene 1-oxide; |
| 1408 | 3-ethyl-2-(3-((5-fluoro-2-methoxy-4-(methylsulfonyl)phenyl)amino)prop-1-yn-1-yl)-7-((1-methylpiperidin- |
| | 4-yl)amino)benzo[b]thiophene 1-oxide; |
| 1409 | 3-ethyl-2-(3-((5-fluoro-2-methoxy-4-(methylsulfonyl)phenyl)amino)prop-1-yn-1-yl)-7-((tetrahydro-2H-pyran- |
| | 4-yl)amino)benzo[b]thiophene 1-oxide; |
| 1410 | 4-((3-(3-(cyanomethyl)-7-((3,3-difluoro-1-methylpiperidin-4-yl)amino)-1-oxidobenzo[b]thiophen-2-yl)prop- |
| | 2-yn-1-yl)amino)-3-methoxy-N-methylbenzamide; |
| 1411 | 4-((3-(3-(cyanomethyl)-7-((1,1-dioxidotetrahydro-2H-thiopyran-4-yl)amino)-1-oxidobenzo[b]thiophen-2-yl) |
| | prop-2-yn-1-yl)amino)-3-methoxy-N-methylbenzamide; |
| 1412 | 4-((3-(3-(cyanomethyl)-7-(((3R,4S)-3-fluoro-1-methylpiperidin-4-yl)amino)-1-oxidobenzo[b]thiophen-2-yl) |
| | prop-2-yn-1-yl)amino)-3-methoxy-N-methylbenzamide; |
| 1413 | 4-((3-(3-(cyanomethyl)-7-((3-fluoro-1-methylpiperidin-4-yl)amino)-1-oxidobenzo[b]thiophen-2-yl)prop-2-yn- |
| | 1-yl)amino)-3-methoxy-N-methylbenzamide; |
| 1414 | 4-((3-(3-(cyanomethyl)-7-((1-(dimethylglycyl)piperidin-4-yl)amino)-1-oxidobenzo[b]thiophen-2-yl)prop-2- |
| | yn-1-yl)amino)-3-methoxy-N-methylbenzamide; |
| 1415 | 4-((3-(3-(cyanomethyl)-7-((3-fluoro-1-(2-hydroxy-3-methoxypropyl)piperidin-4-yl)amino)-1-oxidobenzo[b] |
| | thiophen-2-yl)prop-2-yn-1-yl)amino)-3-methoxy-N-methylbenzamide; |
| 1416 | 4-((3-(3-(cyanomethyl)-7-(((3R,4R)-3-fluoro-1-methylpiperidin-4-yl)amino)-1-oxidobenzo[b]thiophen-2- |
| | yl)prop-2-yn-1-yl)amino)-3-methoxy-N-methylbenzamide; |
| 1417 | 4-((3-(3-(cyanomethyl)-7-(((3S,4S)-3-fluoro-1-methylpiperidin-4-yl)amino)-1-oxidobenzo[b]thiophen-2-yl) |
| | prop-2-yn-1-yl)amino)-3-methoxy-N-methylbenzamide; |
| 1418 | 4-((3-(3-(cyanomethyl)-7-((4-(dimethylamino)cyclohexyl)amino)-1-oxidobenzo[b]thiophen-2-yl)prop-2-yn- |
| | 1-yl)amino)-3-methoxy-N-methylbenzamide; |
| 1419 | 4-((3-(3-(cyanomethyl)-7-((2-(dimethylamino)ethyl)amino)-1-oxidobenzo[b]thiophen-2-yl)prop-2-yn-1-yl) |
| | amino)-3-methoxy-N-methylbenzamide; |
| 1420 | 4-((3-(3-(cyanomethyl)-7-(((3S,4R)-3-fluoro-1-methylpiperidin-4-yl)amino)-1-oxidobenzo[b]thiophen-2-yl) |
| | prop-2-yn-1-yl)amino)-3-methoxy-N-methylbenzamide; |
| 1421 | 4-((3-(3-(cyanomethyl)-7-((1-methylpiperidin-4-yl)amino)-1-oxidobenzo[b]thiophen-2-yl)prop-2-yn-1-yl) |
| | amino)-3-methoxy-N-methylbenzamide; |
| 1422 | 4-((3-(3-(cyanomethyl)-1-oxido-7-((tetrahydro-2H-pyran-4-yl)amino)benzo[b]thiophen-2-yl)prop-2-yn-1- |
| | yl)amino)-3-methoxy-N-methylbenzamide; |
| 1423 | 4-((3-(7-((3,3-difluoro-1-methylpiperidin-4-yl)amino)-1-oxido-3-(thiazol-4-yl)benzo[b]thiophen-2-yl)prop- |
| | 2-yn-1-yl)amino)-3-methoxy-N-methylbenzamide; |
| 1424 | 4-((3-(7-((1,1-dioxidotetrahydro-2H-thiopyran-4-yl)amino)-1-oxido-3-(thiazol-4-yl)benzo[b]thiophen-2-yl) |
| | prop-2-yn-1-yl)amino)-3-methoxy-N-methylbenzamide; |
| 1425 | 4-((3-(7-(((3R,4S)-3-fluoro-1-methylpiperidin-4-yl)amino)-1-oxido-3-(thiazol-4-yl)benzo[b]thiophen-2-yl) |
| | prop-2-yn-1-yl)amino)-3-methoxy-N-methylbenzamide; |
| 1426 | 4-((3-(7-((3-fluoro-1-methylpiperidin-4-yl)amino)-1-oxido-3-(thiazol-4-yl)benzo[b]thiophen-2-yl)prop-2-yn- |
| | 1-yl)amino)-3-methoxy-N-methylbenzamide; |
| 1427 | 4-((3-(7-((1-(dimethylglycyl)piperidin-4-yl)amino)-1-oxido-3-(thiazol-4-yl)benzo[b]thiophen-2-yl)prop-2-yn- |
| | 1-yl)amino)-3-methoxy-N-methylbenzamide; |
| 1428 | 4-((3-(7-((3-fluoro-1-(2-hydroxy-3-methoxypropyl)piperidin-4-yl)amino)-1-oxido-3-(thiazol-4-yl)benzo[b] |
| | thiophen-2-yl)prop-2-yn-1-yl)amino)-3-methoxy-N-methylbenzamide; |
| 1429 | 4-((3-(7-(((3R,4R)-3-fluoro-1-methylpiperidin-4-yl)amino)-1-oxido-3-(thiazol-4-yl)benzo[b]thiophen-2-yl) |
| | prop-2-yn-1-yl)amino)-3-methoxy-N-methylbenzamide; |
| 1430 | 4-((3-(7-(((3S,4S)-3-fluoro-1-methylpiperidin-4-yl)amino)-1-oxido-3-(thiazol-4-yl)benzo[b]thiophen-2-yl) |
| | prop-2-yn-1-yl)amino)-3-methoxy-N-methylbenzamide; |
| 1431 | 4-((3-(7-((4-(dimethylamino)cyclohexyl)amino)-1-oxido-3-(thiazol-4-yl)benzo[b]thiophen-2-yl)prop-2-yn-1- |
| | yl)amino)-3-methoxy-N-methylbenzamide; |
| 1432 | 4-((3-(7-((2-(dimethylamino)ethyl)amino)-1-oxido-3-(thiazol-4-yl)benzo[b]thiophen-2-yl)prop-2-yn-1-yl) |
| | amino)-3-methoxy-N-methylbenzamide; |
| 1433 | 4-((3-(7-(((3S,4R)-3-fluoro-1-methylpiperidin-4-yl)amino)-1-oxido-3-(thiazol-4-yl)benzo[b]thiophen-2-yl) |
| | prop-2-yn-1-yl)amino)-3-methoxy-N-methylbenzamide; |
| 1434 | 3-methoxy-N-methyl-4-((3-(7-((1-methylpiperidin-4-yl)amino)-1-oxido-3-(thiazol-4-yl)benzo[b]thiophen-2- |
| | yl)prop-2-yn-1-yl)amino)benzamide; |
| 1435 | 3-methoxy-N-methyl-4-((3-(1-oxido-7-((tetrahydro-2H-pyran-4-yl)amino)-3-(thiazol-4-yl)benzo[b]thiophen- |
| | 2-yl)prop-2-yn-1-yl)amino)benzamide; |
| 1436 | 4-((3-(7-((3,3-difluoro-1-methylpiperidin-4-yl)amino)-1-oxido-3-(2,2,2-trifluoroethyl)benzo[b]thiophen-2- |
| | yl)prop-2-yn-1-yl)amino)-3-methoxy-N-methylbenzamide; |
| 1437 | 4-((3-(7-((1,1-dioxidotetrahydro-2H-thiopyran-4-yl)amino)-1-oxido-3-(2,2,2-trifluoroethyl)benzo[b]thiophen- |
| | 2-yl)prop-2-yn-1-yl)amino)-3-methoxy-N-methylbenzamide; |
| 1438 | 4-((3-(7-(((3R,4S)-3-fluoro-1-methylpiperidin-4-yl)amino)-1-oxido-3-(2,2,2-trifluoroethyl)benzo[b]thiophen- |
| | 2-yl)prop-2-yn-1-yl)amino)-3-methoxy-N-methylbenzamide; |
| 1439 | 4-((3-(7-((3-fluoro-1-methylpiperidin-4-yl)amino)-1-oxido-3-(2,2,2-trifluoroethyl)benzo[b]thiophen-2-yl)prop- |
| | 2-yn-1-yl)amino)-3-methoxy-N-methylbenzamide; |
| 1440 | 4-((3-(7-((1-(dimethylglycyl)piperidin-4-yl)amino)-1-oxido-3-(2,2,2-trifluoroethyl)benzo[b]thiophen-2-yl)prop- |
| | 2-yn-1-yl)amino)-3-methoxy-N-methylbenzamide; |
| 1441 | 4-((3-(7-((3-fluoro-1-(2-hydroxy-3-methoxypropyl)piperidin-4-yl)amino)-1-oxido-3-(2,2,2-trifluoroethyl)benzo |
| | [b]thiophen-2-yl)prop-2-yn-1-yl)amino)-3-methoxy-N-methylbenzamide; |
| 1442 | 4-((3-(7-(((3R,4R)-3-fluoro-1-methylpiperidin-4-yl)amino)-1-oxido-3-(2,2,2-trifluoroethyl)benzo[b]thiophen- |
| | 2-yl)prop-2-yn-1-yl)amino)-3-methoxy-N-methylbenzamide; |
| 1443 | 4-((3-(7-(((3S,4S)-3-fluoro-1-methylpiperidin-4-yl)amino)-1-oxido-3-(2,2,2-trifluoroethyl)benzo[b]thiophen- |
| | 2-yl)prop-2-yn-1-yl)amino)-3-methoxy-N-methylbenzamide; |
| 1444 | 4-((3-(7-((4-(dimethylamino)cyclohexyl)amino)-1-oxido-3-(2,2,2-trifluoroethyl)benzo[b]thiophen-2-yl)prop- |
| | 2-yn-1-yl)amino)-3-methoxy-N-methylbenzamide; |
| 1445 | 4-((3-(7-((2-(dimethylamino)ethyl)amino)-1-oxido-3-(2,2,2-trifluoroethyl)benzo[b]thiophen-2-yl)prop-2-yn- |
| | 1-yl)amino)-3-methoxy-N-methylbenzamide; |
| 1446 | 4-((3-(7-(((3S,4R)-3-fluoro-1-methylpiperidin-4-yl)amino)-1-oxido-3-(2,2,2-trifluoroethyl)benzo[b]thiophen- |
| | 2-yl)prop-2-yn-1-yl)amino)-3-methoxy-N-methylbenzamide; |
| 1447 | 3-methoxy-N-methyl-4-((3-(7-((1-methylpiperidin-4-yl)amino)-1-oxido-3-(2,2,2-trifluoroethyl)benzo[b]thiophen- |
| | 2-yl)prop-2-yn-1-yl)amino)benzamide; |
| 1448 | 3-methoxy-N-methyl-4-((3-(1-oxido-7-((tetrahydro-2H-pyran-4-yl)amino)-3-(2,2,2-trifluoroethyl)benzo[b]thiophen- |
| | 2-yl)prop-2-yn-1-yl)amino)benzamide; |
| 1449 | 4-((3-(3-(cyclopropylmethyl)-7-((3,3-difluoro-1-methylpiperidin-4-yl)amino)-1-oxidobenzo[b]thiophen-2- |
| | yl)prop-2-yn-1-yl)amino)-3-methoxy-N-methylbenzamide; |
| 1450 | 4-((3-(3-(cyclopropylmethyl)-7-((1,1-dioxidotetrahydro-2H-thiopyran-4-yl)amino)-1-oxidobenzo[b]thiophen- |
| | 2-yl)prop-2-yn-1-yl)amino)-3-methoxy-N-methylbenzamide; |
| 1451 | 4-((3-(3-(cyclopropylmethyl)-7-(((3R,4S)-3-fluoro-1-methylpiperidin-4-yl)amino)-1-oxidobenzo[b]thiophen- |
| | 2-yl)prop-2-yn-1-yl)amino)-3-methoxy-N-methylbenzamide; |
| 1452 | 4-((3-(3-(cyclopropylmethyl)-7-((3-fluoro-1-methylpiperidin-4-yl)amino)-1-oxidobenzo[b]thiophen-2-yl)prop- |
| | 2-yn-1-yl)amino)-3-methoxy-N-methylbenzamide; |
| 1453 | 4-((3-(3-(cyclopropylmethyl)-7-((1-(dimethylglycyl)piperidin-4-yl)amino)-1-oxidobenzo[b]thiophen-2-yl)prop- |
| | 2-yn-1-yl)amino)-3-methoxy-N-methylbenzamide; |
| 1454 | 4-((3-(3-(cyclopropylmethyl)-7-((3-fluoro-1-(2-hydroxy-3-methoxypropyl)piperidin-4-yl)amino)-1-oxidobenzo |
| | [b]thiophen-2-yl)prop-2-yn-1-yl)amino)-3-methoxy-N-methylbenzamide; |
| 1455 | 4-((3-(3-(cyclopropylmethyl)-7-(((3R,4R)-3-fluoro-1-methylpiperidin-4-yl)amino)-1-oxidobenzo[b]thiophen- |
| | 2-yl)prop-2-yn-1-yl)amino)-3-methoxy-N-methylbenzamide; |
| 1456 | 4-((3-(3-(cyclopropylmethyl)-7-(((3S,4S)-3-fluoro-1-methylpiperidin-4-yl)amino)-1-oxidobenzo[b]thiophen- |
| | 2-yl)prop-2-yn-1-yl)amino)-3-methoxy-N-methylbenzamide; |
| 1457 | 4-((3-(3-(cyclopropylmethyl)-7-((4-(dimethylamino)cyclohexyl)amino)-1-oxidobenzo[b]thiophen-2-yl)prop- |
| | 2-yn-1-yl)amino)-3-methoxy-N-methylbenzamide; |
| 1458 | 4-((3-(3-(cyclopropylmethyl)-7-((2-(dimethylamino)ethyl)amino)-1-oxidobenzo[b]thiophen-2-yl)prop-2-yn- |
| | 1-yl)amino)-3-methoxy-N-methylbenzamide; |
| 1459 | 4-((3-(3-(cyclopropylmethyl)-7-(((3S,4R)-3-fluoro-1-methylpiperidin-4-yl)amino)-1-oxidobenzo[b]thiophen- |
| | 2-yl)prop-2-yn-1-yl)amino)-3-methoxy-N-methylbenzamide; |
| 1460 | 4-((3-(3-(cyclopropylmethyl)-7-((1-methylpiperidin-4-yl)amino)-1-oxidobenzo[b]thiophen-2-yl)prop-2-yn- |
| | 1-yl)amino)-3-methoxy-N-methylbenzamide; |
| 1461 | 4-((3-(3-(cyclopropylmethyl)-1-oxido-7-((tetrahydro-2H-pyran-4-yl)amino)benzo[b]thiophen-2-yl)prop-2-yn- |
| | 1-yl)amino)-3-methoxy-N-methylbenzamide; |
| 1462 | 4-((3-(7-((3,3-difluoro-1-methylpiperidin-4-yl)amino)-3-(2,2-difluoroethyl)-1-oxidobenzo[b]thiophen-2-yl) |
| | prop-2-yn-1-yl)amino)-3-methoxy-N-methylbenzamide; |
| 1463 | 4-((3-(3-(2,2-difluoroethyl)-7-((1,1-dioxidotetrahydro-2H-thiopyran-4-yl)amino)-1-oxidobenzo[b]thiophen- |
| | 2-yl)prop-2-yn-1-yl)amino)-3-methoxy-N-methylbenzamide; |
| 1464 | 4-((3-(3-(2,2-difluoroethyl)-7-(((3R,4S)-3-fluoro-1-methylpiperidin-4-yl)amino)-1-oxidobenzo[b]thiophen- |
| | 2-yl)prop-2-yn-1-yl)amino)-3-methoxy-N-methylbenzamide; |
| 1465 | 4-((3-(3-(2,2-difluoroethyl)-7-((3-fluoro-1-methylpiperidin-4-yl)amino)-1-oxidobenzo[b]thiophen-2-yl)prop- |
| | 2-yn-1-yl)amino)-3-methoxy-N-methylbenzamide; |
| 1466 | 4-((3-(3-(2,2-difluoroethyl)-7-((1-(dimethylglycyl)piperidin-4-yl)amino)-1-oxidobenzo[b]thiophen-2-yl)prop- |
| | 2-yn-1-yl)amino)-3-methoxy-N-methylbenzamide; |
| 1467 | 4-((3-(3-(2,2-difluoroethyl)-7-((3-fluoro-1-(2-hydroxy-3-methoxypropyl)piperidin-4-yl)amino)-1-oxidobenzo |
| | [b]thiophen-2-yl)prop-2-yn-1-yl)amino)-3-methoxy-N-methylbenzamide; |
| 1468 | 4-((3-(3-(2,2-difluoroethyl)-7-(((3R,4R)-3-fluoro-1-methylpiperidin-4-yl)amino)-1-oxidobenzo[b]thiophen- |
| | 2-yl)prop-2-yn-1-yl)amino)-3-methoxy-N-methylbenzamide; |
| 1469 | 4-((3-(3-(2,2-difluoroethyl)-7-(((3S,4S)-3-fluoro-1-methylpiperidin-4-yl)amino)-1-oxidobenzo[b]thiophen- |
| | 2-yl)prop-2-yn-1-yl)amino)-3-methoxy-N-methylbenzamide; |
| 1470 | 4-((3-(3-(2,2-difluoroethyl)-7-((4-(dimethylamino)cyclohexyl)amino)-1-oxidobenzo[b]thiophen-2-yl)prop-2- |
| | yn-1-yl)amino)-3-methoxy-N-methylbenzamide; |
| 1471 | 4-((3-(3-(2,2-difluoroethyl)-7-((2-(dimethylamino)ethyl)amino)-1-oxidobenzo[b]thiophen-2-yl)prop-2-yn-1- |
| | yl)amino)-3-methoxy-N-methylbenzamide; |
| 1472 | 4-((3-(3-(2,2-difluoroethyl)-7-(((3S,4R)-3-fluoro-1-methylpiperidin-4-yl)amino)-1-oxidobenzo[b]thiophen- |
| | 2-yl)prop-2-yn-1-yl)amino)-3-methoxy-N-methylbenzamide; |
| 1473 | 4-((3-(3-(2,2-difluoroethyl)-7-((1-methylpiperidin-4-yl)amino)-1-oxidobenzo[b]thiophen-2-yl)prop-2-yn-1- |
| | yl)amino)-3-methoxy-N-methylbenzamide; |
| 1474 | 4-((3-(3-(2,2-difluoroethyl)-1-oxido-7-((tetrahydro-2H-pyran-4-yl)amino)benzo[b]thiophen-2-yl)prop-2-yn- |
| | 1-yl)amino)-3-methoxy-N-methylbenzamide; |
| 1475 | 4-((3-(7-((3,3-difluoro-1-methylpiperidin-4-yl)amino)-1-oxido-3-(1H-pyrrol-1-yl)benzo[b]thiophen-2-yl)prop- |
| | 2-yn-1-yl)amino)-3-methoxy-N-methylbenzamide; |
| 1476 | 4-((3-(7-((1,1-dioxidotetrahydro-2H-thiopyran-4-yl)amino)-1-oxido-3-(1H-pyrrol-1-yl)benzo[b]thiophen-2- |
| | yl)prop-2-yn-1-yl)amino)-3-methoxy-N-methylbenzamide; |
| 1477 | 4-((3-(7-(((3R,4S)-3-fluoro-1-methylpiperidin-4-yl)amino)-1-oxido-3-(1H-pyrrol-1-yl)benzo[b]thiophen-2- |
| | yl)prop-2-yn-1-yl)amino)-3-methoxy-N-methylbenzamide; |
| 1478 | 4-((3-(7-((3-fluoro-1-methylpiperidin-4-yl)amino)-1-oxido-3-(1H-pyrrol-1-yl)benzo[b]thiophen-2-yl)prop-2- |
| | yn-1-yl)amino)-3-methoxy-N-methylbenzamide; |
| 1479 | 4-((3-(7-((1-(dimethylglycyl)piperidin-4-yl)amino)-1-oxido-3-(1H-pyrrol-1-yl)benzo[b]thiophen-2-yl)prop- |
| | 2-yn-1-yl)amino)-3-methoxy-N-methylbenzamide; |
| 1480 | 4-((3-(7-((3-fluoro-1-(2-hydroxy-3-methoxypropyl)piperidin-4-yl)amino)-1-oxido-3-(1H-pyrrol-1-yl)benzo |
| | [b]thiophen-2-yl)prop-2-yn-1-yl)amino)-3-methoxy-N-methylbenzamide; |
| 1481 | 4-((3-(7-(((3R,4R)-3-fluoro-1-methylpiperidin-4-yl)amino)-1-oxido-3-(1H-pyrrol-1-yl)benzo[b]thiophen-2- |
| | yl)prop-2-yn-1-yl)amino)-3-methoxy-N-methylbenzamide; |
| 1482 | 4-((3-(7-(((3S,4S)-3-fluoro-1-methylpiperidin-4-yl)amino)-1-oxido-3-(1H-pyrrol-1-yl)benzo[b]thiophen-2- |
| | yl)prop-2-yn-1-yl)amino)-3-methoxy-N-methylbenzamide; |
| 1483 | 4-((3-(7-((4-(dimethylamino)cyclohexyl)amino)-1-oxido-3-(1H-pyrrol-1-yl)benzo[b]thiophen-2-yl)prop-2-yn- |
| | 1-yl)amino)-3-methoxy-N-methylbenzamide; |
| 1484 | 4-((3-(7-((2-(dimethylamino)ethyl)amino)-1-oxido-3-(1H-pyrrol-1-yl)benzo[b]thiophen-2-yl)prop-2-yn-1- |
| | yl)amino)-3-methoxy-N-methylbenzamide; |
| 1485 | 4-((3-(7-(((3S,4R)-3-fluoro-1-methylpiperidin-4-yl)amino)-1-oxido-3-(1H-pyrrol-1-yl)benzo[b]thiophen-2- |
| | yl)prop-2-yn-1-yl)amino)-3-methoxy-N-methylbenzamide; |
| 1486 | 3-methoxy-N-methyl-4-((3-(7-((1-methylpiperidin-4-yl)amino)-1-oxido-3-(1H-pyrrol-1-yl)benzo[b]thiophen- |
| | 2-yl)prop-2-yn-1-yl)amino)benzamide; |
| 1487 | 3-methoxy-N-methyl-4-((3-(1-oxido-3-(1H-pyrrol-1-yl)-7-((tetrahydro-2H-pyran-4-yl)amino)benzo[b]thiophen- |
| | 2-yl)prop-2-yn-1-yl)amino)benzamide; |
| 1488 | 4-((3-(7-((3,3-difluoro-1-methylpiperidin-4-yl)amino)-3-ethyl-1-oxidobenzo[b]thiophen-2-yl)prop-2-yn-1- |
| | yl)amino)-3-methoxy-N-methylbenzamide; |
| 1489 | 4-((3-(7-((1,1-dioxidotetrahydro-2H-thiopyran-4-yl)amino)-3-ethyl-1-oxidobenzo[b]thiophen-2-yl)prop-2-yn- |
| | 1-yl)amino)-3-methoxy-N-methylbenzamide; |
| 1490 | 4-((3-(3-ethyl-7-(((3R,4S)-3-fluoro-1-methylpiperidin-4-yl)amino)-1-oxidobenzo[b]thiophen-2-yl)prop-2-yn- |
| | 1-yl)amino)-3-methoxy-N-methylbenzamide; |
| 1491 | 4-((3-(3-ethyl-7-((3-fluoro-1-methylpiperidin-4-yl)amino)-1-oxidobenzo[b]thiophen-2-yl)prop-2-yn-1-yl) |
| | amino)-3-methoxy-N-methylbenzamide; |
| 1492 | 4-((3-(7-((1-(dimethylglycyl)piperidin-4-yl)amino)-3-ethyl-1-oxidobenzo[b]thiophen-2-yl)prop-2-yn-1-yl) |
| | amino)-3-methoxy-N-methylbenzamide; |
| 1493 | 4-((3-(3-ethyl-7-((3-fluoro-1-(2-hydroxy-3-methoxypropyl)piperidin-4-yl)amino)-1-oxidobenzo[b]thiophen- |
| | 2-yl)prop-2-yn-1-yl)amino)-3-methoxy-N-methylbenzamide; |
| 1494 | 4-((3-(3-ethyl-7-(((3R,4R)-3-fluoro-1-methylpiperidin-4-yl)amino)-1-oxidobenzo[b]thiophen-2-yl)prop-2-yn- |
| | 1-yl)amino)-3-methoxy-N-methylbenzamide; |
| 1495 | 4-((3-(3-ethyl-7-(((3S,4S)-3-fluoro-1-methylpiperidin-4-yl)amino)-1-oxidobenzo[b]thiophen-2-yl)prop-2-yn- |
| | 1-yl)amino)-3-methoxy-N-methylbenzamide; |
| 1496 | 4-((3-(7-((4-(dimethylamino)cyclohexyl)amino)-3-ethyl-1-oxidobenzo[b]thiophen-2-yl)prop-2-yn-1-yl)amino)- |
| | 3-methoxy-N-methylbenzamide; |
| 1497 | 4-((3-(7-((2-(dimethylamino)ethyl)amino)-3-ethyl-1-oxidobenzo[b]thiophen-2-yl)prop-2-yn-1-yl)amino)-3- |
| | methoxy-N-methylbenzamide; |
| 1498 | 4-((3-(3-ethyl-7-(((3S,4R)-3-fluoro-1-methylpiperidin-4-yl)amino)-1-oxidobenzo[b]thiophen-2-yl)prop-2-yn- |
| | 1-yl)amino)-3-methoxy-N-methylbenzamide; |
| 1499 | 4-((3-(3-ethyl-7-((1-methylpiperidin-4-yl)amino)-1-oxidobenzo[b]thiophen-2-yl)prop-2-yn-1-yl)amino)-3- |
| | methoxy-N-methylbenzamide; |
| 1500 | 4-((3-(3-ethyl-1-oxido-7-((tetrahydro-2H-pyran-4-yl)amino)benzo[b]thiophen-2-yl)prop-2-yn-1-yl)amino)- |
| | 3-methoxy-N-methylbenzamide; |
| 1501 | 2-(7-((3,3-difluoro-1-methylpiperidin-4-yl)amino)-2-(3-((2-methoxy-4-(methylsulfonyl)phenyl)amino)prop- |
| | 1-yn-1-yl)-1-oxidobenzo[b]thiophen-3-yl)acetonitrile; |
| 1502 | 2-(7-((1,1-dioxidotetrahydro-2H-thiopyran-4-yl)amino)-2-(3-((2-methoxy-4-(methylsulfonyl)phenyl)amino) |
| | prop-1-yn-1-yl)-1-oxidobenzo[b]thiophen-3-yl)acetonitrile; |
| 1503 | 2-(7-(((3R,4S)-3-fluoro-1-methylpiperidin-4-yl)amino)-2-(3-((2-methoxy-4-(methylsulfonyl)phenyl)amino) |
| | prop-1-yn-1-yl)-1-oxidobenzo[b]thiophen-3-yl)acetonitrile; |
| 1504 | 2-(7-((3-fluoro-1-methylpiperidin-4-yl)amino)-2-(3-((2-methoxy-4-(methylsulfonyl)phenyl)amino)prop-1-yn- |
| | 1-yl)-1-oxidobenzo[b]thiophen-3-yl)acetonitrile; |
| 1505 | 2-(7-((1-(dimethylglycyl)piperidin-4-yl)amino)-2-(3-((2-methoxy-4-(methylsulfonyl)phenyl)amino)prop-1-yn- |
| | 1-yl)-1-oxidobenzo[b]thiophen-3-yl)acetonitrile; |
| 1506 | 2-(7-((3-fluoro-1-(2-hydroxy-3-methoxypropyl)piperidin-4-yl)amino)-2-(3-((2-methoxy-4-(methylsulfonyl) |
| | phenyl)amino)prop-1-yn-1-yl)-1-oxidobenzo[b]thiophen-3-yl)acetonitrile; |
| 1507 | 2-(7-(((3R,4R)-3-fluoro-1-methylpiperidin-4-yl)amino)-2-(3-((2-methoxy-4-(methylsulfonyl)phenyl)amino) |
| | prop-1-yn-1-yl)-1-oxidobenzo[b]thiophen-3-yl)acetonitrile; |
| 1508 | 2-(7-(((3S,4S)-3-fluoro-1-methylpiperidin-4-yl)amino)-2-(3-((2-methoxy-4-(methylsulfonyl)phenyl)amino) |
| | prop-1-yn-1-yl)-1-oxidobenzo[b]thiophen-3-yl)acetonitrile; |
| 1509 | 2-(7-((4-(dimethylamino)cyclohexyl)amino)-2-(3-((2-methoxy-4-(methylsulfonyl)phenyl)amino)prop-1-yn- |
| | 1-yl)-1-oxidobenzo[b]thiophen-3-yl)acetonitrile; |
| 1510 | 2-(7-((2-(dimethylamino)ethyl)amino)-2-(3-((2-methoxy-4-(methylsulfonyl)phenyl)amino)prop-1-yn-1-yl)- |
| | 1-oxidobenzo[b]thiophen-3-yl)acetonitrile; |
| 1511 | 2-(7-(((3S,4R)-3-fluoro-1-methylpiperidin-4-yl)amino)-2-(3-((2-methoxy-4-(methylsulfonyl)phenyl)amino) |
| | prop-1-yn-1-yl)-1-oxidobenzo[b]thiophen-3-yl)acetonitrile; |
| 1512 | 2-(2-(3-((2-methoxy-4-(methylsulfonyl)phenyl)amino)prop-1-yn-1-yl)-7-((1-methylpiperidin-4-yl)amino)-1- |
| | oxidobenzo[b]thiophen-3-yl)acetonitrile; |
| 1513 | 2-(2-(3-((2-methoxy-4-(methylsulfonyl)phenyl)amino)prop-1-yn-1-yl)-1-oxido-7-((tetrahydro-2H-pyran-4- |
| | yl)amino)benzo[b]thiophen-3-yl)acetonitrile; |
| 1514 | 7-((3,3-difluoro-1-methylpiperidin-4-yl)amino)-2-(3-((2-methoxy-4-(methylsulfonyl)phenyl)amino)prop-1- |
| | yn-1-yl)-3-(thiazol-4-yl)benzo[b]thiophene 1-oxide; |
| 1515 | 4-((2-(3-((2-methoxy-4-(methylsulfonyl)phenyl)amino)prop-1-yn-1-yl)-1-oxido-3-(thiazol-4-yl)benzo[b]thiophen- |
| | 7-yl)amino)tetrahydro-2H-thiopyran 1,1-dioxide; |
| 1516 | 7-(((3R,4S)-3-fluoro-1-methylpiperidin-4-yl)amino)-2-(3-((2-methoxy-4-(methylsulfonyl)phenyl)amino)prop- |
| | 1-yn-1-yl)-3-(thiazol-4-yl)benzo[b]thiophene 1-oxide; |
| 1517 | 7-((3-fluoro-1-methylpiperidin-4-yl)amino)-2-(3-((2-methoxy-4-(methylsulfonyl)phenyl)amino)prop-1-yn-1- |
| | yl)-3-(thiazol-4-yl)benzo[b]thiophene 1-oxide; |
| 1518 | 2-(dimethylamino)-1-(4-((2-(3-((2-methoxy-4-(methylsulfonyl)phenyl)amino)prop-1-yn-1-yl)-1-oxido-3- |
| | (thiazol-4-yl)benzo[b]thiophen-7-yl)amino)piperidin-1-yl)ethan-1-one; |
| 1519 | 7-((3-fluoro-1-(2-hydroxy-3-methoxypropyl)piperidin-4-yl)amino)-2-(3-((2-methoxy-4-(methylsulfonyl) |
| | phenyl)amino)prop-1-yn-1-yl)-3-(thiazol-4-yl)benzo[b]thiophene 1-oxide; |
| 1520 | 7-(((3R,4R)-3-fluoro-1-methylpiperidin-4-yl)amino)-2-(3-((2-methoxy-4-(methylsulfonyl)phenyl)amino)prop- |
| | 1-yn-1-yl)-3-(thiazol-4-yl)benzo[b]thiophene 1-oxide; |
| 1521 | 7-(((3S,4S)-3-fluoro-1-methylpiperidin-4-yl)amino)-2-(3-((2-methoxy-4-(methylsulfonyl)phenyl)amino)prop- |
| | 1-yn-1-yl)-3-(thiazol-4-yl)benzo[b]thiophene 1-oxide; |
| 1522 | 7-((4-(dimethylamino)cyclohexyl)amino)-2-(3-((2-methoxy-4-(methylsulfonyl)phenyl)amino)prop-1-yn-1- |
| | yl)-3-(thiazol-4-yl)benzo[b]thiophene 1-oxide; |
| 1523 | 7-((2-(dimethylamino)ethyl)amino)-2-(3-((2-methoxy-4-(methylsulfonyl)phenyl)amino)prop-1-yn-1-yl)-3- |
| | (thiazol-4-yl)benzo[b]thiophene 1-oxide; |
| 1524 | 7-(((3S,4R)-3-fluoro-1-methylpiperidin-4-yl)amino)-2-(3-((2-methoxy-4-(methylsulfonyl)phenyl)amino)prop- |
| | 1-yn-1-yl)-3-(thiazol-4-yl)benzo[b]thiophene 1-oxide; |
| 1525 | 2-(3-((2-methoxy-4-(methylsulfonyl)phenyl)amino)prop-1-yn-1-yl)-7-((1-methylpiperidin-4-yl)amino)-3- |
| | (thiazol-4-yl)benzo[b]thiophene 1-oxide; |
| 1526 | 2-(3-((2-methoxy-4-(methylsulfonyl)phenyl)amino)prop-1-yn-1-yl)-7-((tetrahydro-2H-pyran-4-yl)amino)- |
| | 3-(thiazol-4-yl)benzo[b]thiophene 1-oxide; |
| 1527 | 7-((3,3-difluoro-1-methylpiperidin-4-yl)amino)-2-(3-((2-methoxy-4-(methylsulfonyl)phenyl)amino)prop-1- |
| | yn-1-yl)-3-(2,2,2-trifluoroethyl)benzo[b]thiophene 1-oxide; |
| 1528 | 4-((2-(3-((2-methoxy-4-(methylsulfonyl)phenyl)amino)prop-1-yn-1-yl)-1-oxido-3-(2,2,2-trifluoroethyl)benzo |
| | [b]thiophen-7-yl)amino)tetrahydro-2H-thiopyran 1,1-dioxide; |
| 1529 | 7-(((3R,4S)-3-fluoro-1-methylpiperidin-4-yl)amino)-2-(3-((2-methoxy-4-(methylsulfonyl)phenyl)amino)prop- |
| | 1-yn-1-yl)-3-(2,2,2-trifluoroethyl)benzo[b]thiophene 1-oxide; |
| 1530 | 7-((3-fluoro-1-methylpiperidin-4-yl)amino)-2-(3-((2-methoxy-4-(methylsulfonyl)phenyl)amino)prop-1-yn-1- |
| | yl)-3-(2,2,2-trifluoroethyl)benzo[b]thiophene 1-oxide; |
| 1531 | 2-(dimethylamino)-1-(4-((2-(3-((2-methoxy-4-(methylsulfonyl)phenyl)amino)prop-1-yn-1-yl)-1-oxido-3- |
| | (2,2,2-trifluoroethyl)benzo[b]thiophen-7-yl)amino)piperidin-1-yl)ethan-1-one; |
| 1532 | 7-((3-fluoro-1-(2-hydroxy-3-methoxypropyl)piperidin-4-yl)amino)-2-(3-((2-methoxy-4-(methylsulfonyl) |
| | phenyl)amino)prop-1-yn-1-yl)-3-(2,2,2-trifluoroethyl)benzo[b]thiophene 1-oxide; |
| 1533 | 7-(((3R,4R)-3-fluoro-1-methylpiperidin-4-yl)amino)-2-(3-((2-methoxy-4-(methylsulfonyl)phenyl)amino)prop- |
| | 1-yn-1-yl)-3-(2,2,2-trifluoroethyl)benzo[b]thiophene 1-oxide; |
| 1534 | 7-(((3S,4S)-3-fluoro-1-methylpiperidin-4-yl)amino)-2-(3-((2-methoxy-4-(methylsulfonyl)phenyl)amino)prop- |
| | 1-yn-1-yl)-3-(2,2,2-trifluoroethyl)benzo[b]thiophene 1-oxide; |
| 1535 | 7-((4-(dimethylamino)cyclohexyl)amino)-2-(3-((2-methoxy-4-(methylsulfonyl)phenyl)amino)prop-1-yn-1- |
| | yl)-3-(2,2,2-trifluoroethyl)benzo[b]thiophene 1-oxide; |
| 1536 | 7-((2-(dimethylamino)ethyl)amino)-2-(3-((2-methoxy-4-(methylsulfonyl)phenyl)amino)prop-1-yn-1-yl)-3- |
| | (2,2,2-trifluoroethyl)benzo[b]thiophene 1-oxide; |
| 1537 | 7-(((3S,4R)-3-fluoro-1-methylpiperidin-4-yl)amino)-2-(3-((2-methoxy-4-(methylsulfonyl)phenyl)amino)prop- |
| | 1-yn-1-yl)-3-(2,2,2-trifluoroethyl)benzo[b]thiophene 1-oxide; |
| 1538 | 2-(3-((2-methoxy-4-(methylsulfonyl)phenyl)amino)prop-1-yn-1-yl)-7-((1-methylpiperidin-4-yl)amino)-3- |
| | (2,2,2-trifluoroethyl)benzo[b]thiophene 1-oxide; |
| 1539 | 2-(3-((2-methoxy-4-(methylsulfonyl)phenyl)amino)prop-1-yn-1-yl)-7-((tetrahydro-2H-pyran-4-yl)amino)-3- |
| | (2,2,2-trifluoroethyl)benzo[b]thiophene 1-oxide; |
| 1540 | 3-(cyclopropylmethyl)-7-((3,3-difluoro-1-methylpiperidin-4-yl)amino)-2-(3-((2-methoxy-4-(methylsulfonyl) |
| | phenyl)amino)prop-1-yn-1-yl)benzo[b]thiophene 1-oxide; |
| 1541 | 4-((3-(cyclopropylmethyl)-2-(3-((2-methoxy-4-(methylsulfonyl)phenyl)amino)prop-1-yn-1-yl)-1-oxidobenzo |
| | [b]thiophen-7-yl)amino)tetrahydro-2H-thiopyran 1,1-dioxide; |
| 1542 | 3-(cyclopropylmethyl)-7-(((3R,4S)-3-fluoro-1-methylpiperidin-4-yl)amino)-2-(3-((2-methoxy-4- |
| | (methylsulfonyl)phenyl)amino)prop-1-yn-1-yl)benzo[b]thiophene 1-oxide; |
| 1543 | 3-(cyclopropylmethyl)-7-((3-fluoro-1-methylpiperidin-4-yl)amino)-2-(3-((2-methoxy-4-(methylsulfonyl) |
| | phenyl)amino)prop-1-yn-1-yl)benzo[b]thiophene 1-oxide; |
| 1544 | 1-(4-((3-(cyclopropylmethyl)-2-(3-((2-methoxy-4-(methylsulfonyl)phenyl)amino)prop-1-yn-1-yl)-1-oxidobenzo |
| | [b]thiophen-7-yl)amino)piperidin-1-yl)-2-(dimethylamino)ethan-1-one; |
| 1545 | 3-(cyclopropylmethyl)-7-((3-fluoro-1-(2-hydroxy-3-methoxypropyl)piperidin-4-yl)amino)-2-(3-((2-methoxy- |
| | 4-(methylsulfonyl)phenyl)amino)prop-1-yn-1-yl)benzo[b]thiophene 1-oxide; |
| 1546 | 3-(cyclopropylmethyl)-7-(((3R,4R)-3-fluoro-1-methylpiperidin-4-yl)amino)-2-(3-((2-methoxy-4-(methylsulfonyl) |
| | phenyl)amino)prop-1-yn-1-yl)benzo[b]thiophene 1-oxide; |
| 1547 | 3-(cyclopropylmethyl)-7-(((3S,4S)-3-fluoro-1-methylpiperidin-4-yl)amino)-2-(3-((2-methoxy-4-(methylsulfonyl) |
| | phenyl)amino)prop-1-yn-1-yl)benzo[b]thiophene 1-oxide; |
| 1548 | 3-(cyclopropylmethyl)-7-((4-(dimethylamino)cyclohexyl)amino)-2-(3-((2-methoxy-4-(methylsulfonyl)phenyl) |
| | amino)prop-1-yn-1-yl)benzo[b]thiophene 1-oxide; |
| 1549 | 3-(cyclopropylmethyl)-7-((2-(dimethylamino)ethyl)amino)-2-(3-((2-methoxy-4-(methylsulfonyl)phenyl)amino) |
| | prop-1-yn-1-yl)benzo[b]thiophene 1-oxide; |
| 1550 | 3-(cyclopropylmethyl)-7-(((3S,4R)-3-fluoro-1-methylpiperidin-4-yl)amino)-2-(3-((2-methoxy-4-(methylsulfonyl) |
| | phenyl)amino)prop-1-yn-1-yl)benzo[b]thiophene 1-oxide; |
| 1551 | 3-(cyclopropylmethyl)-2-(3-((2-methoxy-4-(methylsulfonyl)phenyl)amino)prop-1-yn-1-yl)-7-((1-methylpiperidin- |
| | 4-yl)amino)benzo[b]thiophene 1-oxide; |
| 1552 | 3-(cyclopropylmethyl)-2-(3-((2-methoxy-4-(methylsulfonyl)phenyl)amino)prop-1-yn-1-yl)-7-((tetrahydro-2H- |
| | pyran-4-yl)amino)benzo[b]thiophene 1-oxide; |
| 1553 | 7-((3,3-difluoro-1-methylpiperidin-4-yl)amino)-3-(2,2-difluoroethyl)-2-(3-((2-methoxy-4-(methylsulfonyl) |
| | phenyl)amino)prop-1-yn-1-yl)benzo[b]thiophene 1-oxide; |
| 1554 | 4-((3-(2,2-difluoroethyl)-2-(3-((2-methoxy-4-(methylsulfonyl)phenyl)amino)prop-1-yn-1-yl)-1-oxidobenzo |
| | [b]thiophen-7-yl)amino)tetrahydro-2H-thiopyran 1,1-dioxide; |
| 1555 | 3-(2,2-difluoroethyl)-7-(((3R,4S)-3-fluoro-1-methylpiperidin-4-yl)amino)-2-(3-((2-methoxy-4-(methylsulfonyl) |
| | phenyl)amino)prop-1-yn-1-yl)benzo[b]thiophene 1-oxide; |
| 1556 | 3-(2,2-difluoroethyl)-7-((3-fluoro-1-methylpiperidin-4-yl)amino)-2-(3-((2-methoxy-4-(methylsulfonyl)phenyl) |
| | amino)prop-1-yn-1-yl)benzo[b]thiophene 1-oxide; |
| 1557 | 1-(4-((3-(2,2-difluoroethyl)-2-(3-((2-methoxy-4-(methylsulfonyl)phenyl)amino)prop-1-yn-1-yl)-1-oxidobenzo |
| | [b]thiophen-7-yl)amino)piperidin-1-yl)-2-(dimethylamino)ethan-1-one; |
| 1558 | 3-(2,2-difluoroethyl)-7-((3-fluoro-1-(2-hydroxy-3-methoxypropyl)piperidin-4-yl)amino)-2-(3-((2-methoxy- |
| | 4-(methylsulfonyl)phenyl)amino)prop-1-yn-1-yl)benzo[b]thiophene 1-oxide; |
| 1559 | 3-(2,2-difluoroethyl)-7-(((3R,4R)-3-fluoro-1-methylpiperidin-4-yl)amino)-2-(3-((2-methoxy-4-(methylsulfonyl) |
| | phenyl)amino)prop-1-yn-1-yl)benzo[b]thiophene 1-oxide; |
| 1560 | 3-(2,2-difluoroethyl)-7-(((3S,4S)-3-fluoro-1-methylpiperidin-4-yl)amino)-2-(3-((2-methoxy-4-(methylsulfonyl) |
| | phenyl)amino)prop-1-yn-1-yl)benzo[b]thiophene 1-oxide; |
| 1561 | 3-(2,2-difluoroethyl)-7-((4-(dimethylamino)cyclohexyl)amino)-2-(3-((2-methoxy-4-(methylsulfonyl)phenyl) |
| | amino)prop-1-yn-1-yl)benzo[b]thiophene 1-oxide; |
| 1562 | 3-(2,2-difluoroethyl)-7-((2-(dimethylamino)ethyl)amino)-2-(3-((2-methoxy-4-(methylsulfonyl)phenyl)amino) |
| | prop-1-yn-1-yl)benzo[b]thiophene 1-oxide; |
| 1563 | 3-(2,2-difluoroethyl)-7-(((3S,4R)-3-fluoro-1-methylpiperidin-4-yl)amino)-2-(3-((2-methoxy-4-(methylsulfonyl) |
| | phenyl)amino)prop-1-yn-1-yl)benzo[b]thiophene 1-oxide; |
| 1564 | 3-(2,2-difluoroethyl)-2-(3-((2-methoxy-4-(methylsulfonyl)phenyl)amino)prop-1-yn-1-yl)-7-((1-methylpiperidin- |
| | 4-yl)amino)benzo[b]thiophene 1-oxide; |
| 1565 | 3-(2,2-difluoroethyl)-2-(3-((2-methoxy-4-(methylsulfonyl)phenyl)amino)prop-1-yn-1-yl)-7-((tetrahydro-2H- |
| | pyran-4-yl)amino)benzo[b]thiophene 1-oxide; |
| 1566 | 7-((3,3-difluoro-1-methylpiperidin-4-yl)amino)-2-(3-((2-methoxy-4-(methylsulfonyl)phenyl)amino)prop-1- |
| | yn-1-yl)-3-(1H-pyrrol-1-yl)benzo[b]thiophene 1-oxide; |
| 1567 | 4-((2-(3-((2-methoxy-4-(methylsulfonyl)phenyl)amino)prop-1-yn-1-yl)-1-oxido-3-(1H-pyrrol-1-yl)benzo[b] |
| | thiophen-7-yl)amino)tetrahydro-2H-thiopyran 1,1-dioxide; |
| 1568 | 7-(((3R,4S)-3-fluoro-1-methylpiperidin-4-yl)amino)-2-(3-((2-methoxy-4-(methylsulfonyl)phenyl)amino)prop- |
| | 1-yn-1-yl)-3-(1H-pyrrol-1-yl)benzo[b]thiophene 1-oxide; |
| 1569 | 7-((3-fluoro-1-methylpiperidin-4-yl)amino)-2-(3-((2-methoxy-4-(methylsulfonyl)phenyl)amino)prop-1-yn-1- |
| | yl)-3-(1H-pyrrol-1-yl)benzo[b]thiophene 1-oxide; |
| 1570 | 2-(dimethylamino)-1-(4-((2-(3-((2-methoxy-4-(methylsulfonyl)phenyl)amino)prop-1-yn-1-yl)-1-oxido-3-(1H- |
| | pyrrol-1-yl)benzo[b]thiophen-7-yl)amino)piperidin-1-yl)ethan-1-one; |
| 1571 | 7-((3-fluoro-1-(2-hydroxy-3-methoxypropyl)piperidin-4-yl)amino)-2-(3-((2-methoxy-4-(methylsulfonyl) |
| | phenyl)amino)prop-1-yn-1-yl)-3-(1H-pyrrol-1-yl)benzo[b]thiophene 1-oxide; |
| 1572 | 7-(((3R,4R)-3-fluoro-1-methylpiperidin-4-yl)amino)-2-(3-((2-methoxy-4-(methylsulfonyl)phenyl)amino)prop- |
| | 1-yn-1-yl)-3-(1H-pyrrol-1-yl)benzo[b]thiophene 1-oxide; |
| 1573 | 7-(((3S,4S)-3-fluoro-1-methylpiperidin-4-yl)amino)-2-(3-((2-methoxy-4-(methylsulfonyl)phenyl)amino)prop- |
| | 1-yn-1-yl)-3-(1H-pyrrol-1-yl)benzo[b]thiophene 1-oxide; |
| 1574 | 7-((4-(dimethylamino)cyclohexyl)amino)-2-(3-((2-methoxy-4-(methylsulfonyl)phenyl)amino)prop-1-yn-1-yl)- |
| | 3-(1H-pyrrol-1-yl)benzo[b]thiophene 1-oxide; |
| 1575 | 7-((2-(dimethylamino)ethyl)amino)-2-(3-((2-methoxy-4-(methylsulfonyl)phenyl)amino)prop-1-yn-1-yl)-3- |
| | (1H-pyrrol-1-yl)benzo[b]thiophene 1-oxide; |
| 1576 | 7-(((3S,4R)-3-fluoro-1-methylpiperidin-4-yl)amino)-2-(3-((2-methoxy-4-(methylsulfonyl)phenyl)amino)prop- |
| | 1-yn-1-yl)-3-(1H-pyrrol-1-yl)benzo[b]thiophene 1-oxide; |
| 1577 | 2-(3-((2-methoxy-4-(methylsulfonyl)phenyl)amino)prop-1-yn-1-yl)-7-((1-methylpiperidin-4-yl)amino)-3- |
| | (1H-pyrrol-1-yl)benzo[b]thiophene 1-oxide; |
| 1578 | 2-(3-((2-methoxy-4-(methylsulfonyl)phenyl)amino)prop-1-yn-1-yl)-3-(1H-pyrrol-1-yl)-7-((tetrahydro-2H- |
| | pyran-4-yl)amino)benzo[b]thiophene 1-oxide; |
| 1579 | 7-((3,3-difluoro-1-methylpiperidin-4-yl)amino)-3-ethyl-2-(3-((2-methoxy-4-(methylsulfonyl)phenyl)amino) |
| | prop-1-yn-1-yl)benzo[b]thiophene 1-oxide; |
| 1580 | 4-((3-ethyl-2-(3-((2-methoxy-4-(methylsulfonyl)phenyl)amino)prop-1-yn-1-yl)-1-oxidobenzo[b]thiophen-7- |
| | yl)amino)tetrahydro-2H-thiopyran 1,1-dioxide; |
| 1581 | 3-ethyl-7-(((3R,4S)-3-fluoro-1-methylpiperidin-4-yl)amino)-2-(3-((2-methoxy-4-(methylsulfonyl)phenyl) |
| | amino)prop-1-yn-1-yl)benzo[b]thiophene 1-oxide; |
| 1582 | 3-ethyl-7-((3-fluoro-1-methylpiperidin-4-yl)amino)-2-(3-((2-methoxy-4-(methylsulfonyl)phenyl)amino)prop- |
| | 1-yn-1-yl)benzo[b]thiophene 1-oxide; |
| 1583 | 2-(dimethylamino)-1-(4-((3-ethyl-2-(3-((2-methoxy-4-(methylsulfonyl)phenyl)amino)prop-1-yn-1-yl)-1- |
| | oxidobenzo[b]thiophen-7-yl)amino)piperidin-1-yl)ethan-1-one; |
| 1584 | 3-ethyl-7-((3-fluoro-1-(2-hydroxy-3-methoxypropyl)piperidin-4-yl)amino)-2-(3-((2-methoxy-4-(methylsulfonyl) |
| | phenyl)amino)prop-1-yn-1-yl)benzo[b]thiophene 1-oxide; |
| 1585 | 3-ethyl-7-(((3R,4R)-3-fluoro-1-methylpiperidin-4-yl)amino)-2-(3-((2-methoxy-4-(methylsulfonyl)phenyl) |
| | amino)prop-1-yn-1-yl)benzo[b]thiophene 1-oxide; |
| 1586 | 3-ethyl-7-(((3S,4S)-3-fluoro-1-methylpiperidin-4-yl)amino)-2-(3-((2-methoxy-4-(methylsulfonyl)phenyl) |
| | amino)prop-1-yn-1-yl)benzo[b]thiophene 1-oxide; |
| 1587 | 7-((4-(dimethylamino)cyclohexyl)amino)-3-ethyl-2-(3-((2-methoxy-4-(methylsulfonyl)phenyl)amino)prop- |
| | 1-yn-1-yl)benzo[b]thiophene 1-oxide; |
| 1588 | 7-((2-(dimethylamino)ethyl)amino)-3-ethyl-2-(3-((2-methoxy-4-(methylsulfonyl)phenyl)amino)prop-1-yn-1- |
| | yl)benzo[b]thiophene 1-oxide; |
| 1589 | 3-ethyl-7-(((3S,4R)-3-fluoro-1-methylpiperidin-4-yl)amino)-2-(3-((2-methoxy-4-(methylsulfonyl)phenyl) |
| | amino)prop-1-yn-1-yl)benzo[b]thiophene 1-oxide; |
| 1590 | 3-ethyl-2-(3-((2-methoxy-4-(methylsulfonyl)phenyl)amino)prop-1-yn-1-yl)-7-((1-methylpiperidin-4-yl)amino) |
| | benzo[b]thiophene 1-oxide; |
| 1591 | 3-ethyl-2-(3-((2-methoxy-4-(methylsulfonyl)phenyl)amino)prop-1-yn-1-yl)-7-((tetrahydro-2H-pyran-4-yl) |
| | amino)benzo[b]thiophene 1-oxide; |
| 1592 | 4-((3-(3-(cyclopropylmethyl)-7-((3,3-difluoro-1-methylpiperidin-4-yl)amino)thieno[3,2-c]pyridin-2-yl)prop- |
| | 2-yn-1-yl)amino)benzenesulfonamide; |
| 1593 | 4-((3-(3-(cyclopropylmethyl)-7-((3-fluoro-1-(2-hydroxy-3-methoxypropyl)piperidin-4-yl)amino)thieno[3,2- |
| | c]pyridin-2-yl)prop-2-yn-1-yl)amino)benzenesulfonamide; |
| 1594 | 4-((3-(3-(cyclopropylmethyl)-7-((3-fluoro-1-methylpiperidin-4-yl)amino)thieno[3,2-c]pyridin-2-yl)prop-2-yn- |
| | 1-yl)amino)benzenesulfonamide; |
| 1595 | 4-((3-(3-(cyclopropylmethyl)-7-((4-(dimethylamino)cyclohexyl)amino)thieno[3,2-c]pyridin-2-yl)prop-2-yn- |
| | 1-yl)amino)benzenesulfonamide; |
| 1596 | 4-((3-(3-(cyclopropylmethyl)-7-((1-methylpiperidin-4-yl)amino)thieno[3,2-c]pyridin-2-yl)prop-2-yn-1-yl) |
| | amino)benzenesulfonamide; |
| 1597 | 4-((3-(7-((3,3-difluoro-1-methylpiperidin-4-yl)amino)-3-ethylthieno[3,2-c]pyridin-2-yl)prop-2-yn-1-yl) |
| | amino)benzenesulfonamide; |
| 1598 | 4-((3-(3-ethyl-7-((3-fluoro-1-(2-hydroxy-3-methoxypropyl)piperidin-4-yl)amino)thieno[3,2-c]pyridin-2-yl) |
| | prop-2-yn-1-yl)amino)benzenesulfonamide; |
| 1599 | 4-((3-(3-ethyl-7-((3-fluoro-1-methylpiperidin-4-yl)amino)thieno[3,2-c]pyridin-2-yl)prop-2-yn-1-yl)amino) |
| | benzenesulfonamide; |
| 1600 | 4-((3-(7-((4-(dimethylamino)cyclohexyl)amino)-3-ethylthieno[3,2-c]pyridin-2-yl)prop-2-yn-1-yl)amino) |
| | benzenesulfonamide; |
| 1601 | 4-((3-(3-ethyl-7-((1-methylpiperidin-4-yl)amino)thieno[3,2-c]pyridin-2-yl)prop-2-yn-1-yl)amino) |
| | benzenesulfonamide; |
| 1602 | 4-((3-(7-((3,3-difluoro-1-methylpiperidin-4-yl)amino)-3-(2,2,2-trifluoroethyl)thieno[3,2-c]pyridin-2-yl)prop- |
| | 2-yn-1-yl)amino)benzenesulfonamide; |
| 1603 | 4-((3-(7-((3-fluoro-1-(2-hydroxy-3-methoxypropyl)piperidin-4-yl)amino)-3-(2,2,2-trifluoroethyl)thieno[3,2- |
| | c]pyridin-2-yl)prop-2-yn-1-yl)amino)benzenesulfonamide; |
| 1604 | 4-((3-(7-((3-fluoro-1-methylpiperidin-4-yl)amino)-3-(2,2,2-trifluoroethyl)thieno[3,2-c]pyridin-2-yl)prop-2- |
| | yn-1-yl)amino)benzenesulfonamide; |
| 1605 | 4-((3-(7-((4-(dimethylamino)cyclohexyl)amino)-3-(2,2,2-trifluoroethyl)thieno[3,2-c]pyridin-2-yl)prop-2-yn- |
| | 1-yl)amino)benzenesulfonamide; |
| 1606 | 4-((3-(7-((1-methylpiperidin-4-yl)amino)-3-(2,2,2-trifluoroethyl)thieno[3,2-c]pyridin-2-yl)prop-2-yn-1-yl) |
| | amino)benzenesulfonamide; |
| 1607 | 4-((3-(3-(cyclopropylmethyl)-7-((3,3-difluoro-1-methylpiperidin-4-yl)amino)thieno[3,2-b]pyridin-2-yl)prop- |
| | 2-yn-1-yl)amino)benzenesulfonamide; |
| 1608 | 4-((3-(7-((3,3-difluoro-1-methylpiperidin-4-yl)amino)-3-ethylthieno[3,2-b]pyridin-2-yl)prop-2-yn-1- |
| | yl)amino)benzenesulfonamide; |
| 1609 | 4-((3-(7-((3,3-difluoro-1-methylpiperidin-4-yl)amino)-3-(2,2,2-trifluoroethyl)thieno[3,2-b]pyridin-2- |
| | yl)prop-2-yn-1-yl)amino)benzenesulfonamide; |
| 1610 | 4-((3-(3-(cyclopropylmethyl)-7-((3-fluoro-1-(2-hydroxy-3-methoxypropyl)piperidin-4-yl)amino)thieno[3,2- |
| | b]pyridin-2-yl)prop-2-yn-1-yl)amino)benzenesulfonamide; |
| 1611 | 4-((3-(3-ethyl-7-((3-fluoro-1-(2-hydroxy-3-methoxypropyl)piperidin-4-yl)amino)thieno[3,2-b]pyridin-2-yl) |
| | prop-2-yn-1-yl)amino)benzenesulfonamide; |
| 1612 | 4-((3-(7-((3-fluoro-1-(2-hydroxy-3-methoxypropyl)piperidin-4-yl)amino)-3-(2,2,2-trifluoroethyl)thieno[3,2- |
| | b]pyridin-2-yl)prop-2-yn-1-yl)amino)benzenesulfonamide; |
| 1613 | 4-((3-(3-(cyclopropylmethyl)-7-((3-fluoro-1-methylpiperidin-4-yl)amino)thieno[3,2-b]pyridin-2-yl)prop-2- |
| | yn-1-yl)amino)benzenesulfonamide; |
| 1614 | 4-((3-(3-ethyl-7-((3-fluoro-1-methylpiperidin-4-yl)amino)thieno[3,2-b]pyridin-2-yl)prop-2-yn-1-yl)amino) |
| | benzenesulfonamide; |
| 1615 | 4-((3-(7-((3-fluoro-1-methylpiperidin-4-yl)amino)-3-(2,2,2-trifluoroethyl)thieno[3,2-b]pyridin-2-yl)prop-2- |
| | yn-1-yl)amino)benzenesulfonamide; |
| 1616 | 4-((3-(3-(cyclopropylmethyl)-7-((4-(dimethylamino)cyclohexyl)amino)thieno[3,2-b]pyridin-2-yl)prop-2-yn- |
| | 1-yl)amino)benzenesulfonamide; |
| 1617 | 4-((3-(7-((4-(dimethylamino)cyclohexyl)amino)-3-ethylthieno[3,2-b]pyridin-2-yl)prop-2-yn-1-yl)amino) |
| | benzenesulfonamide; |
| 1618 | 4-((3-(7-((4-(dimethylamino)cyclohexyl)amino)-3-(2,2,2-trifluoroethyl)thieno[3,2-b]pyridin-2-yl)prop-2-yn- |
| | 1-yl)amino)benzenesulfonamide; |
| 1619 | 4-((3-(3-(cyclopropylmethyl)-7-((1-methylpiperidin-4-yl)amino)thieno[3,2-b]pyridin-2-yl)prop-2-yn-1-yl) |
| | amino)benzenesulfonamide; |
| 1620 | 4-((3-(3-ethyl-7-((1-methylpiperidin-4-yl)amino)thieno[3,2-b]pyridin-2-yl)prop-2-yn-1-yl)amino) |
| | benzenesulfonamide; |
| 1621 | 4-((3-(7-((1-methylpiperidin-4-yl)amino)-3-(2,2,2-trifluoroethyl)thieno[3,2-b]pyridin-2-yl)prop-2-yn-1-yl) |
| | amino)benzenesulfonamide; |
| 1622 | 4-((3-(3-(cyclopropylmethyl)-7-((3,3-difluoro-1-methylpiperidin-4-yl)amino)thieno[2,3-c]pyridin-2-yl)prop- |
| | 2-yn-1-yl)amino)benzenesulfonamide; |
| 1623 | 4-((3-(3-(cyclopropylmethyl)-7-((3-fluoro-1-(2-hydroxy-3-methoxypropyl)piperidin-4-yl)amino)thieno[2,3- |
| | c]pyridin-2-yl)prop-2-yn-1-yl)amino)benzenesulfonamide; |
| 1624 | 4-((3-(3-(cyclopropylmethyl)-7-((3-fluoro-1-methylpiperidin-4-yl)amino)thieno[2,3-c]pyridin-2-yl)prop-2-yn- |
| | 1-yl)amino)benzenesulfonamide; |
| 1625 | 4-((3-(3-(cyclopropylmethyl)-7-((4-(dimethylamino)cyclohexyl)amino)thieno[2,3-c]pyridin-2-yl)prop-2-yn- |
| | 1-yl)amino)benzenesulfonamide; |
| 1626 | 4-((3-(3-(cyclopropylmethyl)-7-((1-methylpiperidin-4-yl)amino)thieno[2,3-c]pyridin-2-yl)prop-2-yn-1-yl) |
| | amino)benzenesulfonamide; |
| 1627 | 4-((3-(7-((3,3-difluoro-1-methylpiperidin-4-yl)amino)-3-ethylthieno[2,3-c]pyridin-2-yl)prop-2-yn-1-yl) |
| | amino)benzenesulfonamide; |
| 1628 | 4-((3-(3-ethyl-7-((3-fluoro-1-(2-hydroxy-3-methoxypropyl)piperidin-4-yl)amino)thieno[2,3-c]pyridin-2-yl) |
| | prop-2-yn-1-yl)amino)benzenesulfonamide; |
| 1629 | 4-((3-(3-ethyl-7-((3-fluoro-1-methylpiperidin-4-yl)amino)thieno[2,3-c]pyridin-2-yl)prop-2-yn-1-yl)amino) |
| | benzenesulfonamide; |
| 1630 | 4-((3-(7-((4-(dimethylamino)cyclohexyl)amino)-3-ethylthieno[2,3-c]pyridin-2-yl)prop-2-yn-1-yl)amino) |
| | benzenesulfonamide; |
| 1631 | 4-((3-(3-ethyl-7-((1-methylpiperidin-4-yl)amino)thieno[2,3-c]pyridin-2-yl)prop-2-yn-1-yl)amino) |
| | benzenesulfonamide; |
| 1632 | 4-((3-(7-((3,3-difluoro-1-methylpiperidin-4-yl)amino)-3-(2,2,2-trifluoroethyl)thieno[2,3-c]pyridin-2-yl)prop- |
| | 2-yn-1-yl)amino)benzenesulfonamide; |
| 1633 | 4-((3-(7-((3-fluoro-1-(2-hydroxy-3-methoxypropyl)piperidin-4-yl)amino)-3-(2,2,2-trifluoroethyl)thieno[2,3- |
| | c]pyridin-2-yl)prop-2-yn-1-yl)amino)benzenesulfonamide; |
| 1634 | 4-((3-(7-((3-fluoro-1-methylpiperidin-4-yl)amino)-3-(2,2,2-trifluoroethyl)thieno[2,3-c]pyridin-2-yl)prop-2- |
| | yn-1-yl)amino)benzenesulfonamide; |
| 1635 | 4-((3-(7-((4-(dimethylamino)cyclohexyl)amino)-3-(2,2,2-trifluoroethyl)thieno[2,3-c]pyridin-2-yl)prop-2-yn- |
| | 1-yl)amino)benzenesulfonamide; |
| 1636 | 4-((3-(7-((1-methylpiperidin-4-yl)amino)-3-(2,2,2-trifluoroethyl)thieno[2,3-c]pyridin-2-yl)prop-2-yn-1-yl) |
| | amino)benzenesulfonamide; |
| 1637 | 4-((3-(3-(cyclopropylmethyl)-7-((4-(dimethylamino)cyclohexyl)amino)benzo[b]thiophen-2-yl)prop-2-yn-1- |
| | yl)amino)benzenesulfonamide; |
| 1638 | 4-((3-(3-(cyclopropylmethyl)-7-((3,3-difluoro-1-methylpiperidin-4-yl)amino)benzo[b]thiophen-2-yl)prop-2- |
| | yn-1-yl)amino)benzenesulfonamide; |
| 1639 | 4-((3-(3-(cyclopropylmethyl)-7-((1-methylpiperidin-4-yl)amino)benzo[b]thiophen-2-yl)prop-2-yn-1-yl)amino) |
| | benzenesulfonamide; |
| 1640 | 4-((3-(3-(cyclopropylmethyl)-7-((3-fluoro-1-methylpiperidin-4-yl)amino)benzo[b]thiophen-2-yl)prop-2-yn- |
| | 1-yl)amino)benzenesulfonamide; |
| 1641 | 4-((3-(3-(cyclopropylmethyl)-7-((3-fluoro-1-(2-hydroxy-3-methoxypropyl)piperidin-4-yl)amino)benzo[b] |
| | thiophen-2-yl)prop-2-yn-1-yl)amino)benzenesulfonamide; |
| 1642 | 4-((3-(7-((4-(dimethylamino)cyclohexyl)amino)-3-ethylbenzo[b]thiophen-2-yl)prop-2-yn-1-yl)amino) |
| | benzenesulfonamide; |
| 1643 | 4-((3-(7-((3,3-difluoro-1-methylpiperidin-4-yl)amino)-3-ethylbenzo[b]thiophen-2-yl)prop-2-yn-1-yl)amino) |
| | benzenesulfonamide; |
| 1644 | 4-((3-(3-ethyl-7-((1-methylpiperidin-4-yl)amino)benzo[b]thiophen-2-yl)prop-2-yn-1-yl)amino) |
| | benzenesulfonamide; |
| 1645 | 4-((3-(3-ethyl-7-((3-fluoro-1-methylpiperidin-4-yl)amino)benzo[b]thiophen-2-yl)prop-2-yn-1-yl)amino) |
| | benzenesulfonamide; |
| 1646 | 4-((3-(3-ethyl-7-((3-fluoro-1-(2-hydroxy-3-methoxypropyl)piperidin-4-yl)amino)benzo[b]thiophen-2-yl) |
| | prop-2-yn-1-yl)amino)benzenesulfonamide; |
| 1647 | 4-((3-(3-(cyclopropylmethyl)-7-((4-(dimethylamino)cyclohexyl)amino)benzofuran-2-yl)prop-2-yn-1-yl)amino) |
| | benzenesulfonamide; |
| 1648 | 4-((3-(7-((4-(dimethylamino)cyclohexyl)amino)-3-ethylbenzofuran-2-yl)prop-2-yn-1-yl)amino) |
| | benzenesulfonamide; |
| 1649 | 4-((3-(3-(cyclopropylmethyl)-7-((3,3-difluoro-1-methylpiperidin-4-yl)amino)benzofuran-2-yl)prop-2-yn-1- |
| | yl)amino)benzenesulfonamide; |
| 1650 | 4-((3-(7-((3,3-difluoro-1-methylpiperidin-4-yl)amino)-3-ethylbenzofuran-2-yl)prop-2-yn-1-yl)amino) |
| | benzenesulfonamide; |
| 1651 | 4-((3-(3-(cyclopropylmethyl)-7-((1-methylpiperidin-4-yl)amino)benzofuran-2-yl)prop-2-yn-1-yl)amino) |
| | benzenesulfonamide; |
| 1652 | 4-((3-(3-ethyl-7-((1-methylpiperidin-4-yl)amino)benzofuran-2-yl)prop-2-yn-1-yl)amino)benzenesulfonamide; |
| 1653 | 4-((3-(3-(cyclopropylmethyl)-7-((3-fluoro-1-methylpiperidin-4-yl)amino)benzofuran-2-yl)prop-2-yn-1-yl) |
| | amino)benzenesulfonamide; |
| 1654 | 4-((3-(3-ethyl-7-((3-fluoro-1-methylpiperidin-4-yl)amino)benzofuran-2-yl)prop-2-yn-1-yl)amino) |
| | benzenesulfonamide; |
| 1655 | 4-((3-(3-(cyclopropylmethyl)-7-((3-fluoro-1-(2-hydroxy-3-methoxypropyl)piperidin-4-yl)amino)benzofuran- |
| | 2-yl)prop-2-yn-1-yl)amino)benzenesulfonamide; |
| 1656 | 4-((3-(3-ethyl-7-((3-fluoro-1-(2-hydroxy-3-methoxypropyl)piperidin-4-yl)amino)benzofuran-2-yl)prop-2-yn- |
| | 1-yl)amino)benzenesulfonamide; |
| 1657 | 4-((3-(3-(cyclopropylmethyl)-7-((4-(dimethylamino)cyclohexyl)amino)-1-oxidobenzo[b]thiophen-2-yl)prop- |
| | 2-yn-1-yl)amino)benzenesulfonamide; |
| 1658 | 4-((3-(3-(cyclopropylmethyl)-7-((3,3-difluoro-1-methylpiperidin-4-yl)amino)-1-oxidobenzo[b]thiophen-2- |
| | yl)prop-2-yn-1-yl)amino)benzenesulfonamide; |
| 1659 | 4-((3-(3-(cyclopropylmethyl)-7-((1-methylpiperidin-4-yl)amino)-1-oxidobenzo[b]thiophen-2-yl)prop-2-yn- |
| | 1-yl)amino)benzenesulfonamide; |
| 1660 | 4-((3-(3-(cyclopropylmethyl)-7-((3-fluoro-1-methylpiperidin-4-yl)amino)-1-oxidobenzo[b]thiophen-2-yl)prop- |
| | 2-yn-1-yl)amino)benzenesulfonamide; |
| 1661 | 4-((3-(3-(cyclopropylmethyl)-7-((3-fluoro-1-(2-hydroxy-3-methoxypropyl)piperidin-4-yl)amino)-1- |
| | oxidobenzo[b]thiophen-2-yl)prop-2-yn-1-yl)amino)benzenesulfonamide; |
| 1662 | 4-((3-(7-((4-(dimethylamino)cyclohexyl)amino)-3-ethyl-1-oxidobenzo[b]thiophen-2-yl)prop-2-yn-1-yl) |
| | amino)benzenesulfonamide; |
| 1663 | 4-((3-(7-((3,3-difluoro-1-methylpiperidin-4-yl)amino)-3-ethyl-1-oxidobenzo[b]thiophen-2-yl)prop-2-yn-1- |
| | yl)amino)benzenesulfonamide; |
| 1664 | 4-((3-(3-ethyl-7-((1-methylpiperidin-4-yl)amino)-1-oxidobenzo[b]thiophen-2-yl)prop-2-yn-1-yl)amino) |
| | benzenesulfonamide; |
| 1665 | 4-((3-(3-ethyl-7-((3-fluoro-1-methylpiperidin-4-yl)amino)-1-oxidobenzo[b]thiophen-2-yl)prop-2-yn-1-yl) |
| | amino)benzenesulfonamide; |
| 1666 | 4-((3-(3-ethyl-7-((3-fluoro-1-(2-hydroxy-3-methoxypropyl)piperidin-4-yl)amino)-1-oxidobenzo[b]thiophen- |
| | 2-yl)prop-2-yn-1-yl)amino)benzenesulfonamide; |
| 1667 | 4-((3-(3-(cyclopropylmethyl)-7-((4-(dimethylamino)cyclohexyl)amino)-1,1-dioxidobenzo[b]thiophen-2-yl) |
| | prop-2-yn-1-yl)amino)benzenesulfonamide; |
| 1668 | 4-((3-(3-(cyclopropylmethyl)-7-((3,3-difluoro-1-methylpiperidin-4-yl)amino)-1,1-dioxidobenzo[b]thiophen- |
| | 2-yl)prop-2-yn-1-yl)amino)benzenesulfonamide; |
| 1669 | 4-((3-(3-(cyclopropylmethyl)-7-((1-methylpiperidin-4-yl)amino)-1,1-dioxidobenzo[b]thiophen-2-yl)prop-2- |
| | yn-1-yl)amino)benzenesulfonamide; |
| 1670 | 4-((3-(3-(cyclopropylmethyl)-7-((3-fluoro-1-methylpiperidin-4-yl)amino)-1,1-dioxidobenzo[b]thiophen-2- |
| | yl)prop-2-yn-1-yl)amino)benzenesulfonamide; |
| 1671 | 4-((3-(3-(cyclopropylmethyl)-7-((3-fluoro-1-(2-hydroxy-3-methoxypropyl)piperidin-4-yl)amino)-1,1- |
| | dioxidobenzo[b]thiophen-2-yl)prop-2-yn-1-yl)amino)benzenesulfonamide; |
| 1672 | 4-((3-(7-((4-(dimethylamino)cyclohexyl)amino)-3-ethyl-1,1-dioxidobenzo[b]thiophen-2-yl)prop-2-yn-1-yl) |
| | amino)benzenesulfonamide; |
| 1673 | 4-((3-(7-((3,3-difluoro-1-methylpiperidin-4-yl)amino)-3-ethyl-1,1-dioxidobenzo[b]thiophen-2-yl)prop-2- |
| | yn-1-yl)amino)benzenesulfonamide; |
| 1674 | 4-((3-(3-ethyl-7-((1-methylpiperidin-4-yl)amino)-1,1-dioxidobenzo[b]thiophen-2-yl)prop-2-yn-1-yl)amino) |
| | benzenesulfonamide; |
| 1675 | 4-((3-(3-ethyl-7-((3-fluoro-1-methylpiperidin-4-yl)amino)-1,1-dioxidobenzo[b]thiophen-2-yl)prop-2-yn-1- |
| | yl)amino)benzenesulfonamide; |
| 1676 | 4-((3-(3-ethyl-7-((3-fluoro-1-(2-hydroxy-3-methoxypropyl)piperidin-4-yl)amino)-1,1-dioxidobenzo[b]thiophen- |
| | 2-yl)prop-2-yn-1-yl)amino)benzenesulfonamide; |
| 1677 | 4-((3-(7-((4-(dimethylamino)cyclohexyl)amino)-3-(2,2,2-trifluoroethyl)benzo[b]thiophen-2-yl)prop-2-yn-1- |
| | yl)amino)benzenesulfonamide; |
| 1678 | 4-((3-(7-((3,3-difluoro-1-methylpiperidin-4-yl)amino)-3-(2,2,2-trifluoroethyl)benzo[b]thiophen-2-yl)prop-2- |
| | yn-1-yl)amino)benzenesulfonamide; |
| 1679 | 4-((3-(7-((1-methylpiperidin-4-yl)amino)-3-(2,2,2-trifluoroethyl)benzo[b]thiophen-2-yl)prop-2-yn-1-yl)amino) |
| | benzenesulfonamide; |
| 1680 | 4-((3-(7-((3-fluoro-1-methylpiperidin-4-yl)amino)-3-(2,2,2-trifluoroethyl)benzo[b]thiophen-2-yl)prop-2-yn- |
| | 1-yl)amino)benzenesulfonamide; |
| 1681 | 4-((3-(7-((3-fluoro-1-(2-hydroxy-3-methoxypropyl)piperidin-4-yl)amino)-3-(2,2,2-trifluoroethyl)benzo[b] |
| | thiophen-2-yl)prop-2-yn-1-yl)amino)benzenesulfonamide; |
| 1682 | 4-((3-(7-((4-(dimethylamino)cyclohexyl)amino)-3-(2,2,2-trifluoroethyl)benzofuran-2-yl)prop-2-yn-1-yl) |
| | amino)benzenesulfonamide; |
| 1683 | 4-((3-(7-((3,3-difluoro-1-methylpiperidin-4-yl)amino)-3-(2,2,2-trifluoroethyl)benzofuran-2-yl)prop-2-yn-1- |
| | yl)amino)benzenesulfonamide; |
| 1684 | 4-((3-(7-((1-methylpiperidin-4-yl)amino)-3-(2,2,2-trifluoroethyl)benzofuran-2-yl)prop-2-yn-1-yl)amino) |
| | benzenesulfonamide; |
| 1685 | 4-((3-(7-((3-fluoro-1-methylpiperidin-4-yl)amino)-3-(2,2,2-trifluoroethyl)benzofuran-2-yl)prop-2-yn-1-yl) |
| | amino)benzenesulfonamide; |
| 1686 | 4-((3-(7-((3-fluoro-1-(2-hydroxy-3-methoxypropyl)piperidin-4-yl)amino)-3-(2,2,2-trifluoroethyl)benzofuran- |
| | 2-yl)prop-2-yn-1-yl)amino)benzenesulfonamide; |
| 1687 | 4-((3-(7-((4-(dimethylamino)cyclohexyl)amino)-1-oxido-3-(2,2,2-trifluoroethyl)benzo[b]thiophen-2-yl)prop- |
| | 2-yn-1-yl)amino)benzenesulfonamide; |
| 1688 | 4-((3-(7-((3,3-difluoro-1-methylpiperidin-4-yl)amino)-1-oxido-3-(2,2,2-trifluoroethyl)benzo[b]thiophen-2- |
| | yl)prop-2-yn-1-yl)amino)benzenesulfonamide; |
| 1689 | 4-((3-(7-((1-methylpiperidin-4-yl)amino)-1-oxido-3-(2,2,2-trifluoroethyl)benzo[b]thiophen-2-yl)prop-2-yn- |
| | 1-yl)amino)benzenesulfonamide; |
| 1690 | 4-((3-(7-((3-fluoro-1-methylpiperidin-4-yl)amino)-1-oxido-3-(2,2,2-trifluoroethyl)benzo[b]thiophen-2-yl) |
| | prop-2-yn-1-yl)amino)benzenesulfonamide; |
| 1691 | 4-((3-(7-((3-fluoro-1-(2-hydroxy-3-methoxypropyl)piperidin-4-yl)amino)-1-oxido-3-(2,2,2-trifluoroethyl) |
| | benzo[b]thiophen-2-yl)prop-2-yn-1-yl)amino)benzenesulfonamide; |
| 1692 | 4-((3-(7-((4-(dimethylamino)cyclohexyl)amino)-1,1-dioxido-3-(2,2,2-trifluoroethyl)benzo[b]thiophen-2-yl) |
| | prop-2-yn-1-yl)amino)benzenesulfonamide; |
| 1693 | 4-((3-(7-((3,3-difluoro-1-methylpiperidin-4-yl)amino)-1,1-dioxido-3-(2,2,2-trifluoroethyl)benzo[b]thiophen- |
| | 2-yl)prop-2-yn-1-yl)amino)benzenesulfonamide; |
| 1694 | 4-((3-(7-((1-methylpiperidin-4-yl)amino)-1,1-dioxido-3-(2,2,2-trifluoroethyl)benzo[b]thiophen-2-yl)prop-2- |
| | yn-1-yl)amino)benzenesulfonamide; |
| 1695 | 4-((3-(7-((3-fluoro-1-methylpiperidin-4-yl)amino)-1,1-dioxido-3-(2,2,2-trifluoroethyl)benzo[b]thiophen-2- |
| | yl)prop-2-yn-1-yl)amino)benzenesulfonamide; or |
| 1696 | 4-((3-(7-((3-fluoro-1-(2-hydroxy-3-methoxypropyl)piperidin-4-yl)amino)-1,1-dioxido-3-(2,2,2-trifluoroethyl) |
| | benzo[b]thiophen-2-yl)prop-2-yn-1-yl)amino)benzenesulfonamide. |

[55]. The compound according to any one of [1] to [53], wherein the compound of formula (I) is selected

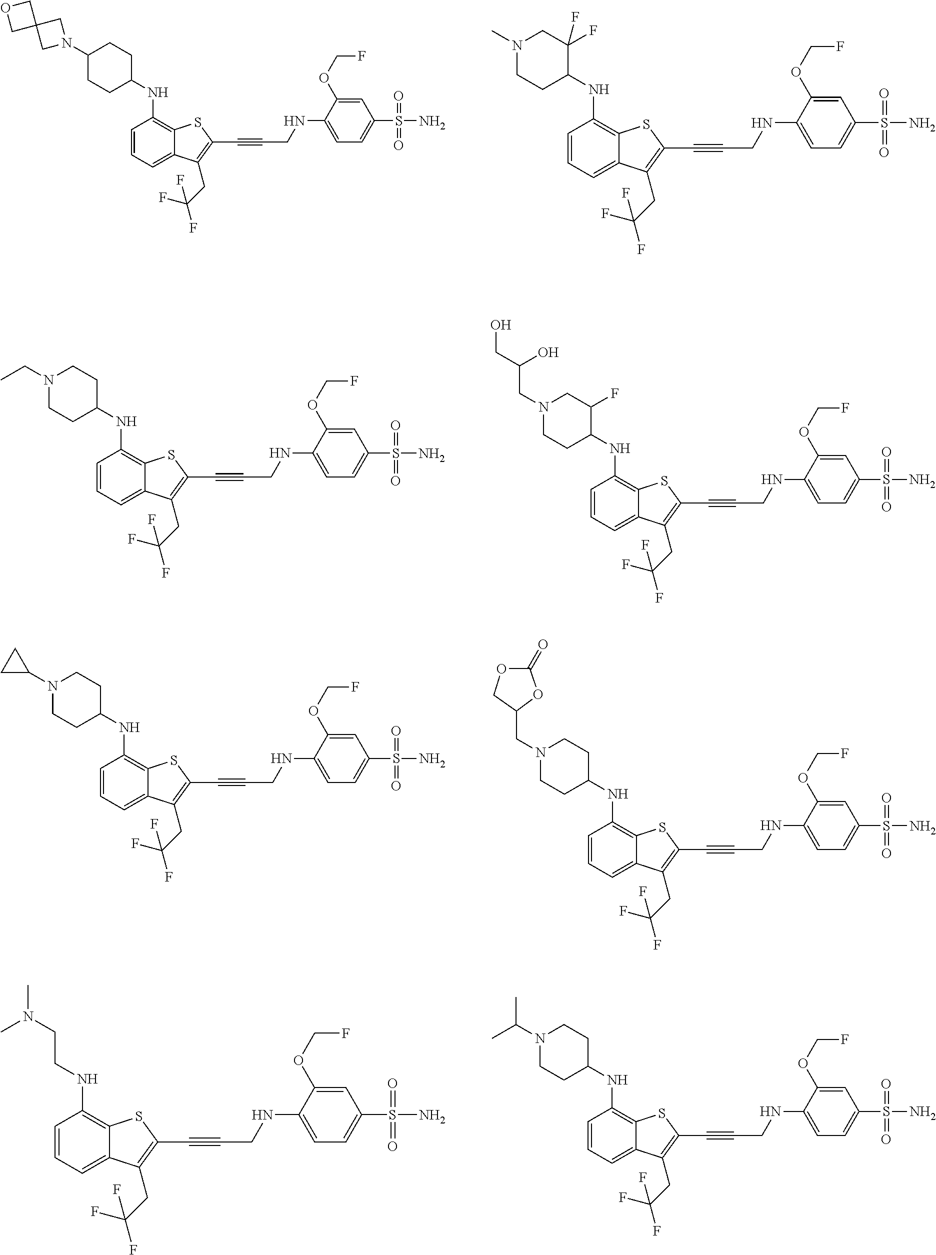

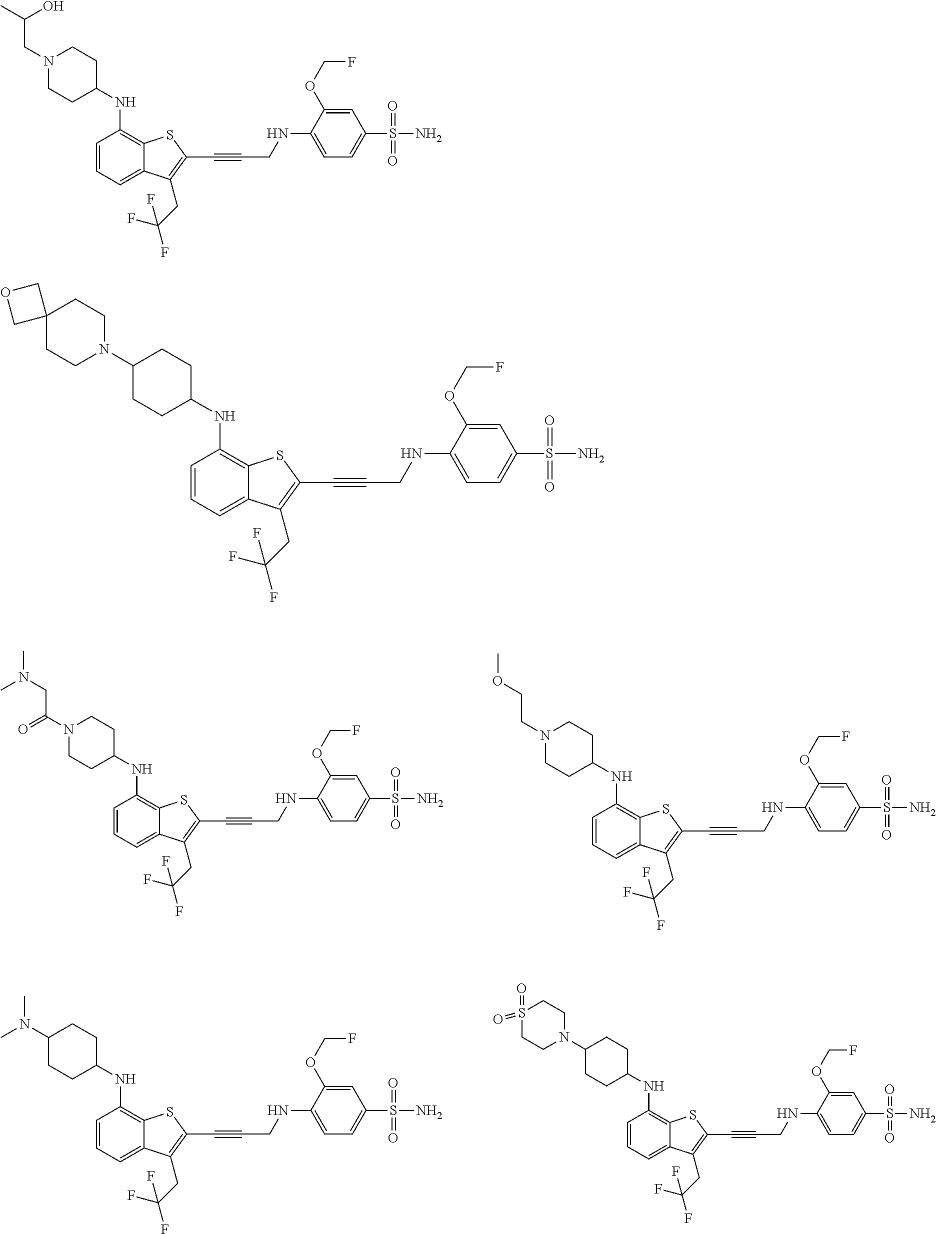 [SMILES: CC(O)CN1CCC(Nc2cccc3c(CC(F)(F)F)c(C#CCNc4ccc(S(N)(=O)=O)cc4OCF)sc23)CC1 ; NS(=O)(=O)c1ccc(NCC#Cc2sc3c(NC4CCC(N5CCC6(CC5)COC6)CC4)cccc3c2CC(F)(F)F)c(OCF)c1 ; CN(C)CC(=O)N1CCC(Nc2cccc3c(CC(F)(F)F)c(C#CCNc4ccc(S(N)(=O)=O)cc4OCF)sc23)CC1 ; COCCN1CCC(Nc2cccc3c(CC(F)(F)F)c(C#CCNc4ccc(S(N)(=O)=O)cc4OCF)sc23)CC1 ; CN(C)C1CCC(Nc2cccc3c(CC(F)(F)F)c(C#CCNc4ccc(S(N)(=O)=O)cc4OCF)sc23)CC1 ; NS(=O)(=O)c1ccc(NCC#Cc2sc3c(NC4CCC(N5CCS(=O)(=O)CC5)CC4)cccc3c2CC(F)(F)F)c(OCF)c1]

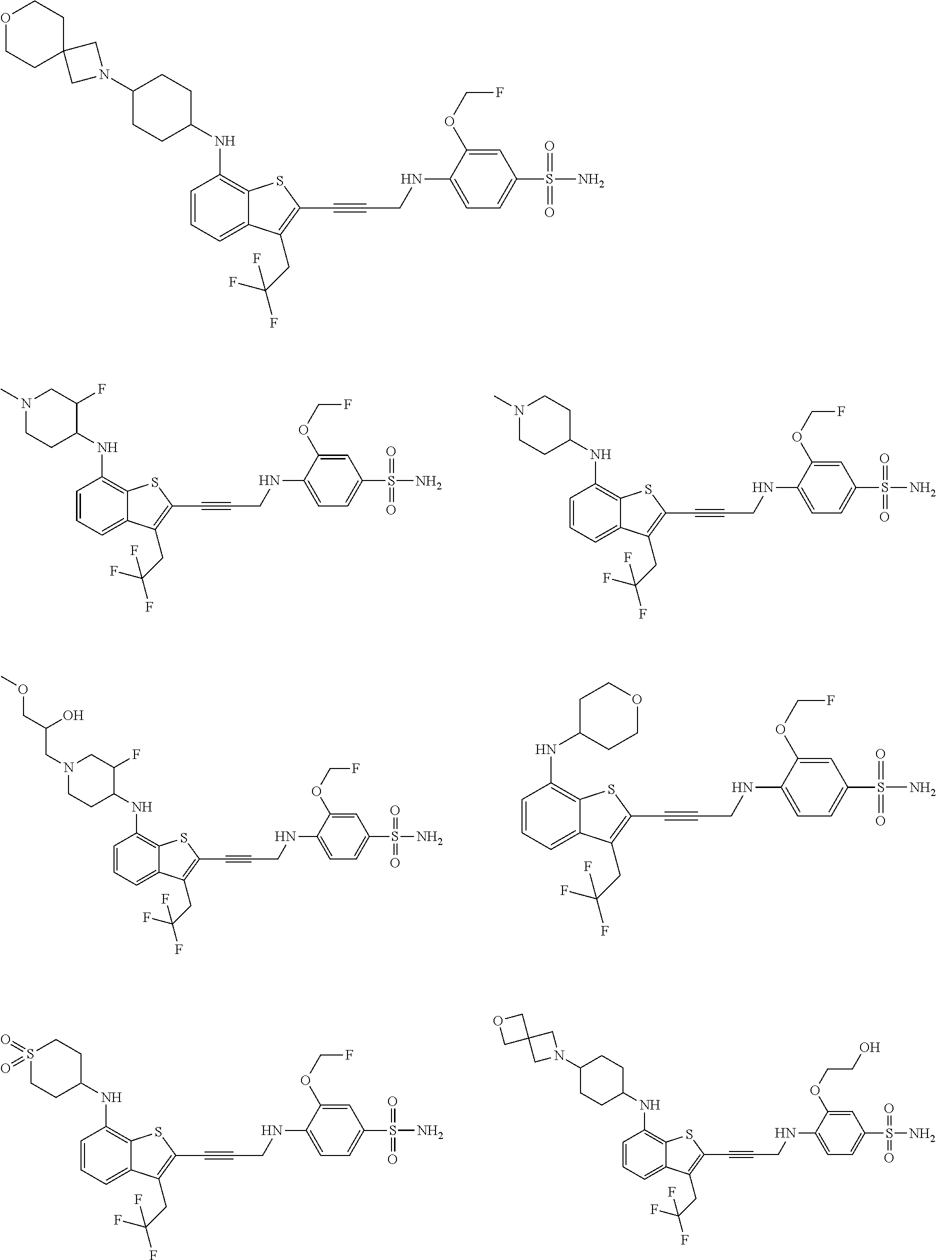

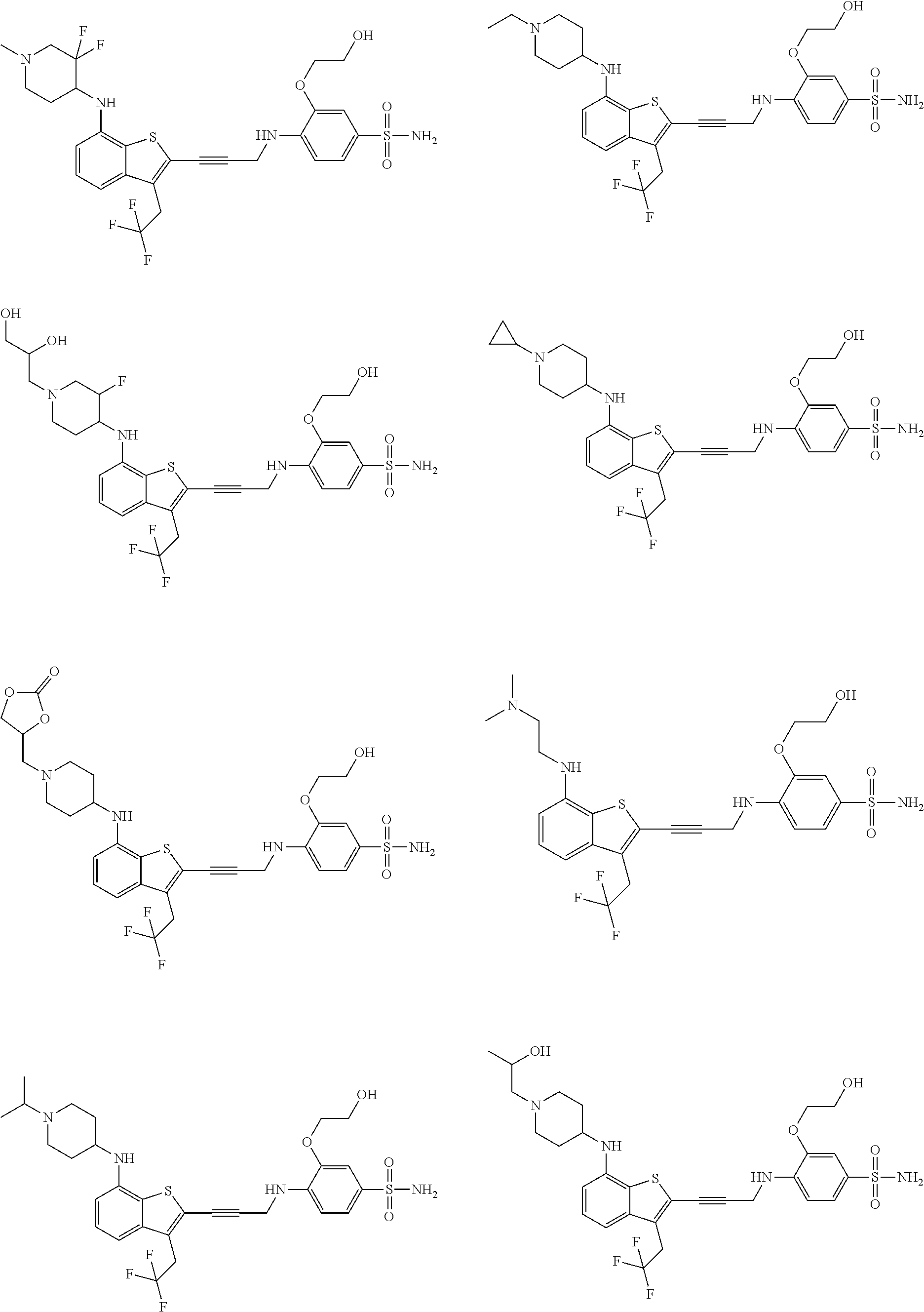

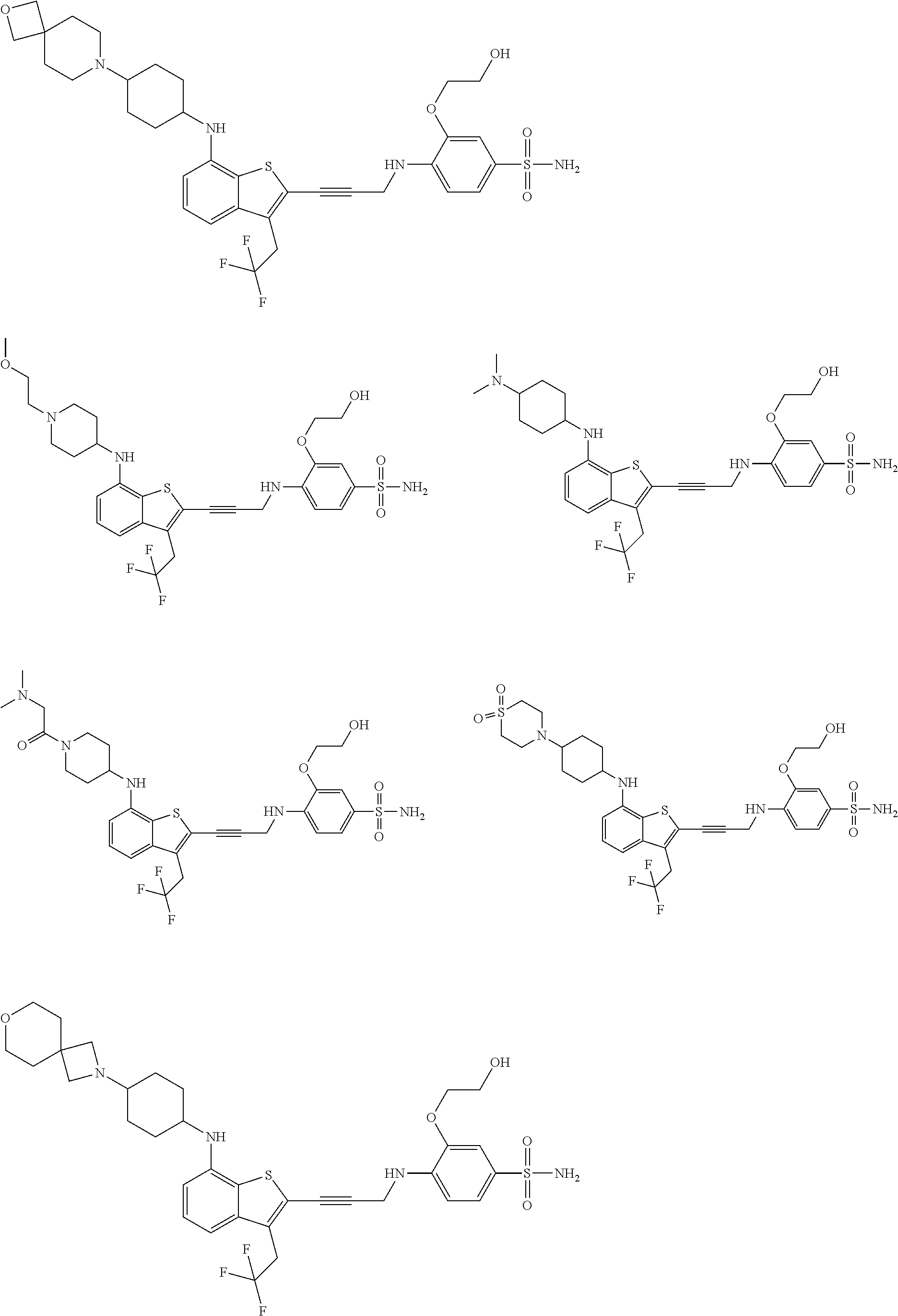 [SMILES: NS(=O)(=O)c1ccc(NCC#Cc2sc3c(NC4CCC(N5CCC6(CC5)COC6)CC4)cccc3c2CC(F)(F)F)c(OCCO)c1 ; COCCN1CCC(Nc2cccc3c(CC(F)(F)F)c(C#CCNc4ccc(S(N)(=O)=O)cc4OCCO)sc23)CC1 ; CN(C)C1CCC(Nc2cccc3c(CC(F)(F)F)c(C#CCNc4ccc(S(N)(=O)=O)cc4OCCO)sc23)CC1 ; CN(C)CC(=O)N1CCC(Nc2cccc3c(CC(F)(F)F)c(C#CCNc4ccc(S(N)(=O)=O)cc4OCCO)sc23)CC1 ; NS(=O)(=O)c1ccc(NCC#Cc2sc3c(NC4CCC(N5CCS(=O)(=O)CC5)CC4)cccc3c2CC(F)(F)F)c(OCCO)c1 ; NS(=O)(=O)c1ccc(NCC#Cc2sc3c(NC4CCC(N5CC6(CCOCC6)C5)CC4)cccc3c2CC(F)(F)F)c(OCCO)c1]

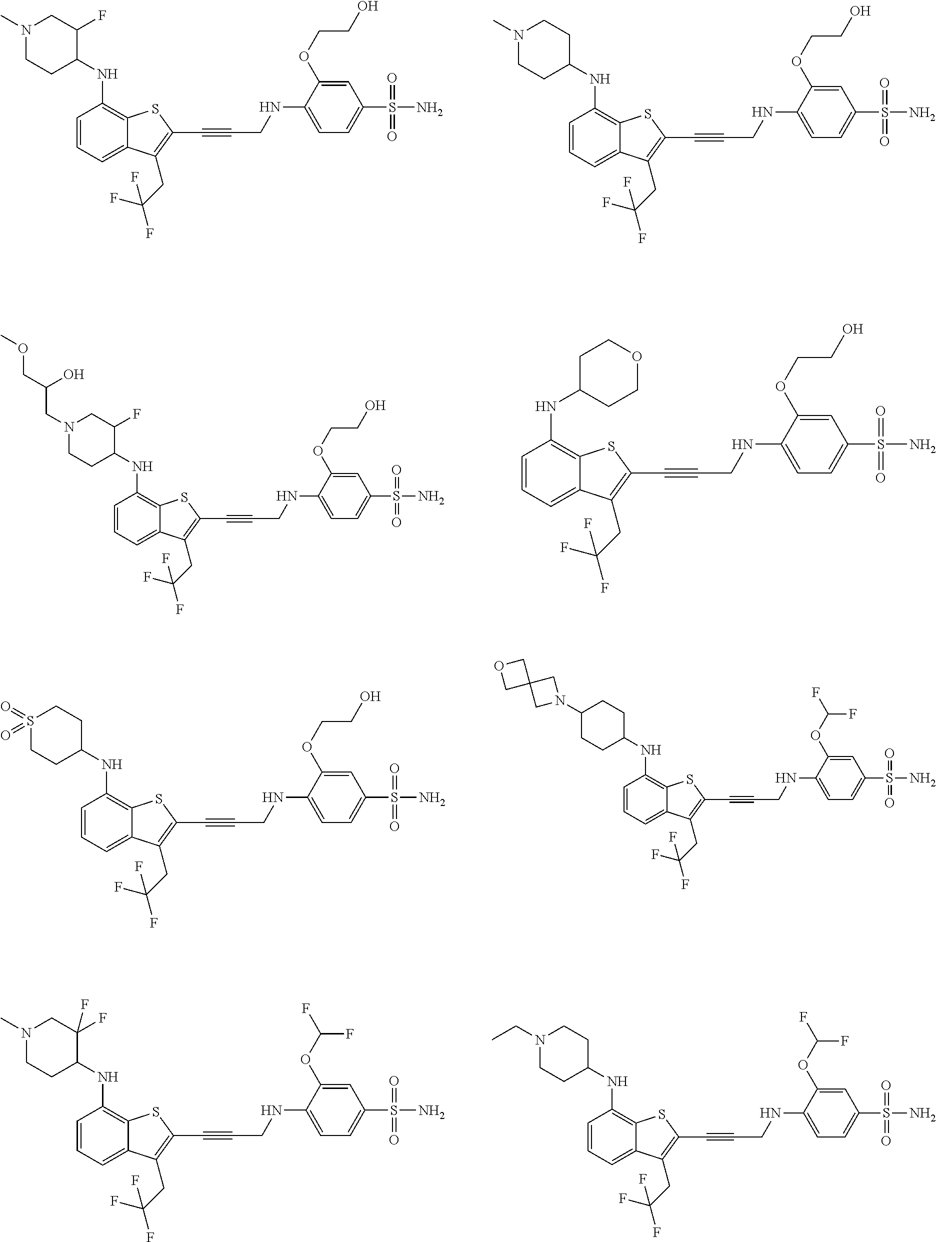

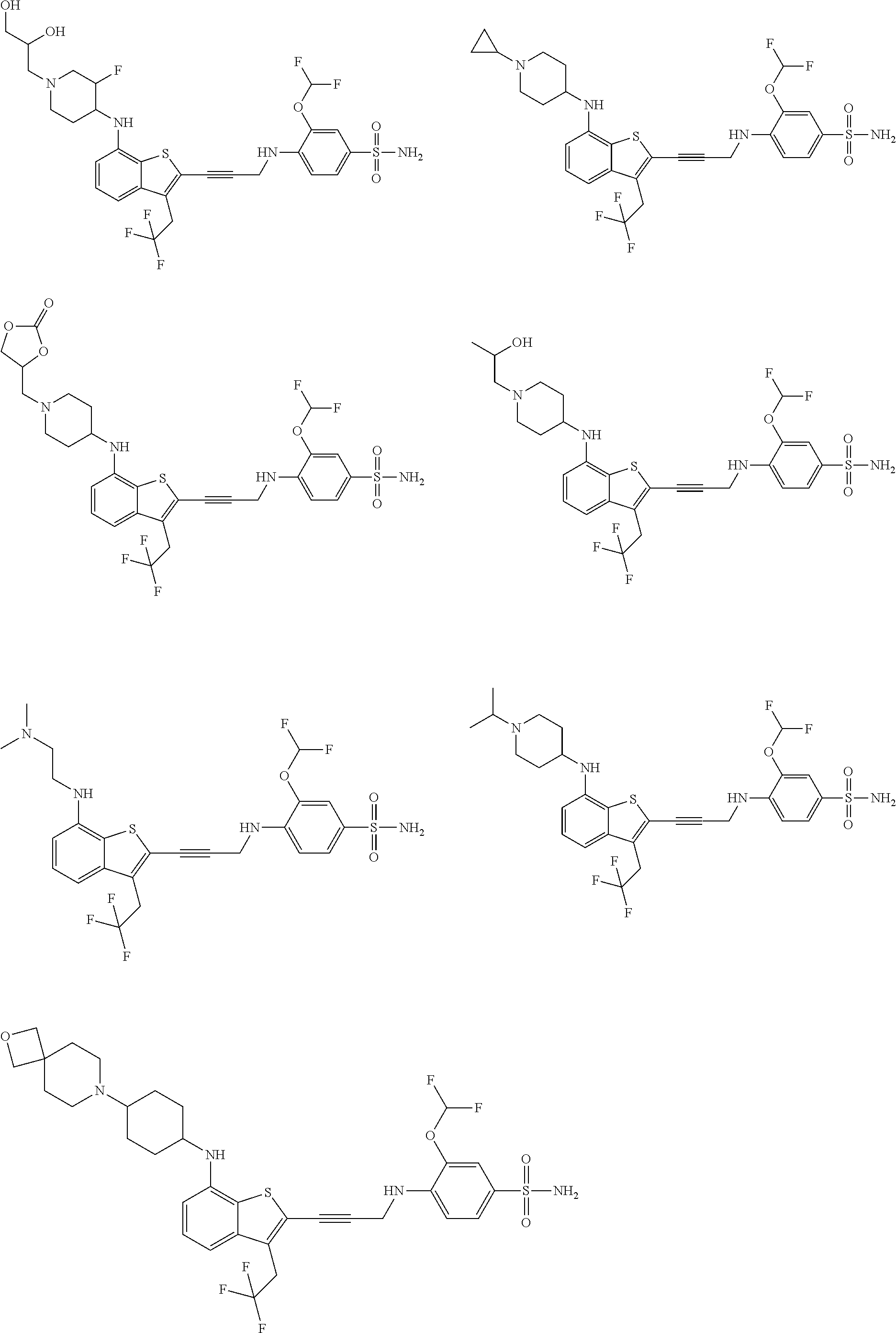

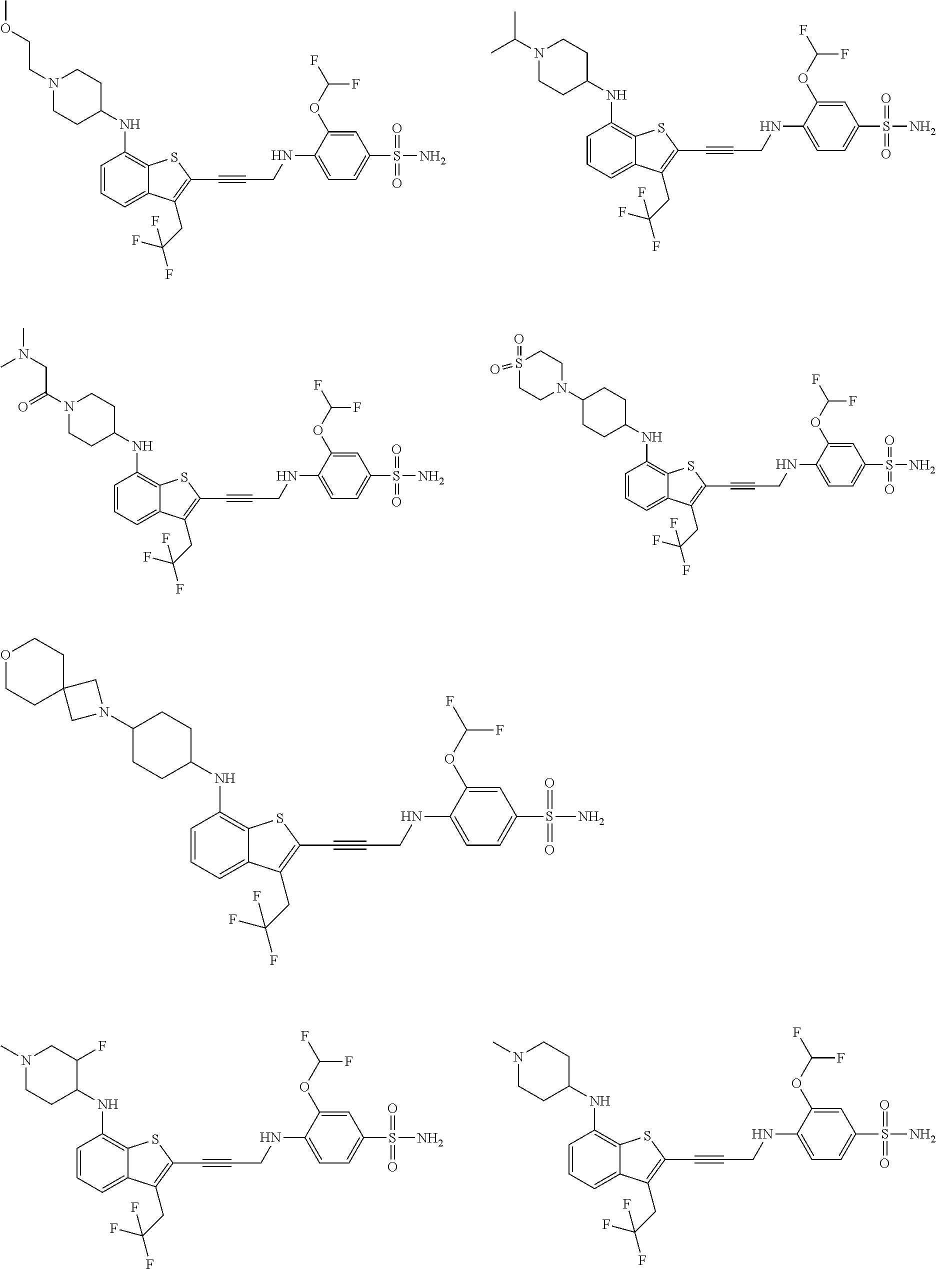

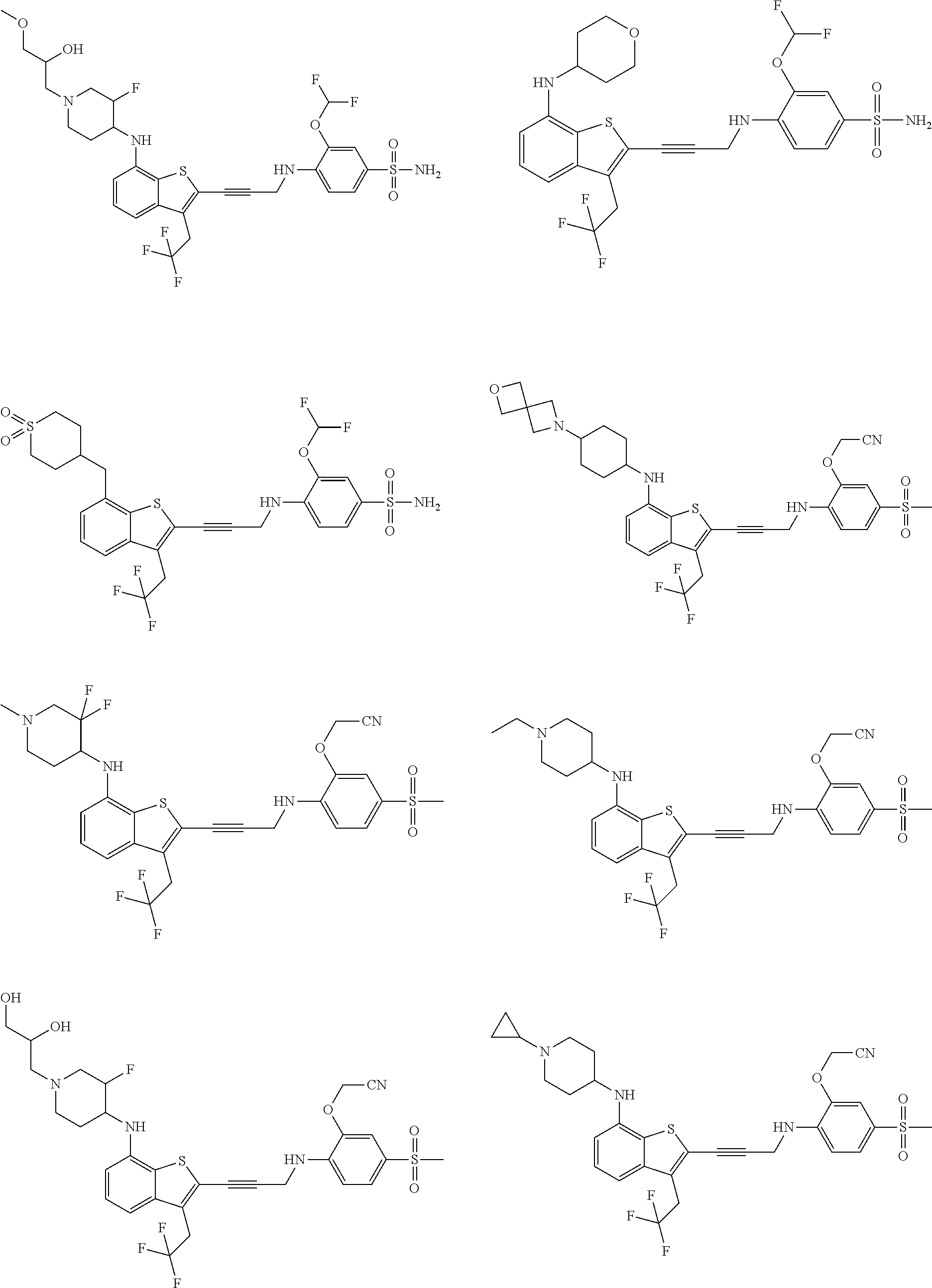

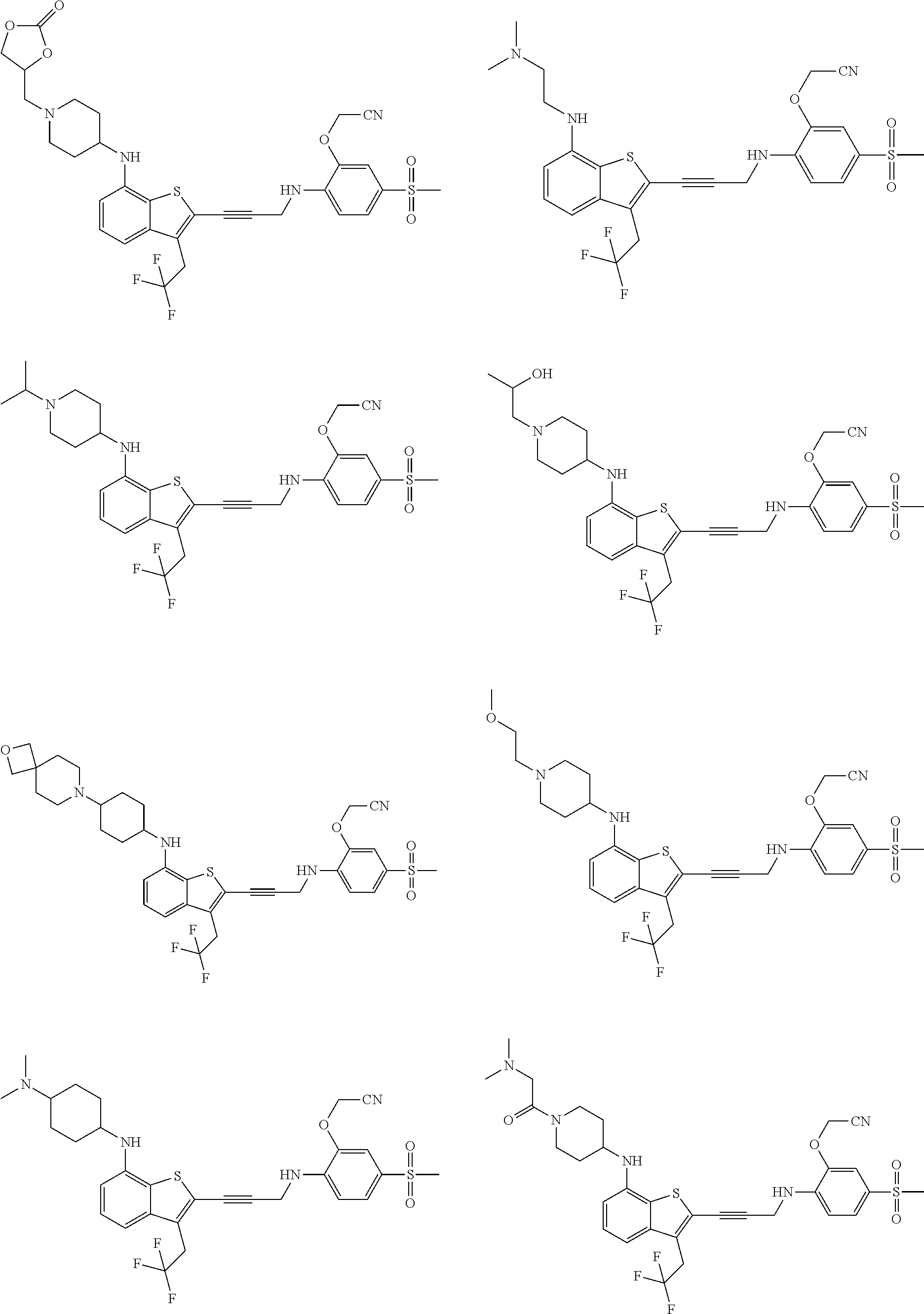

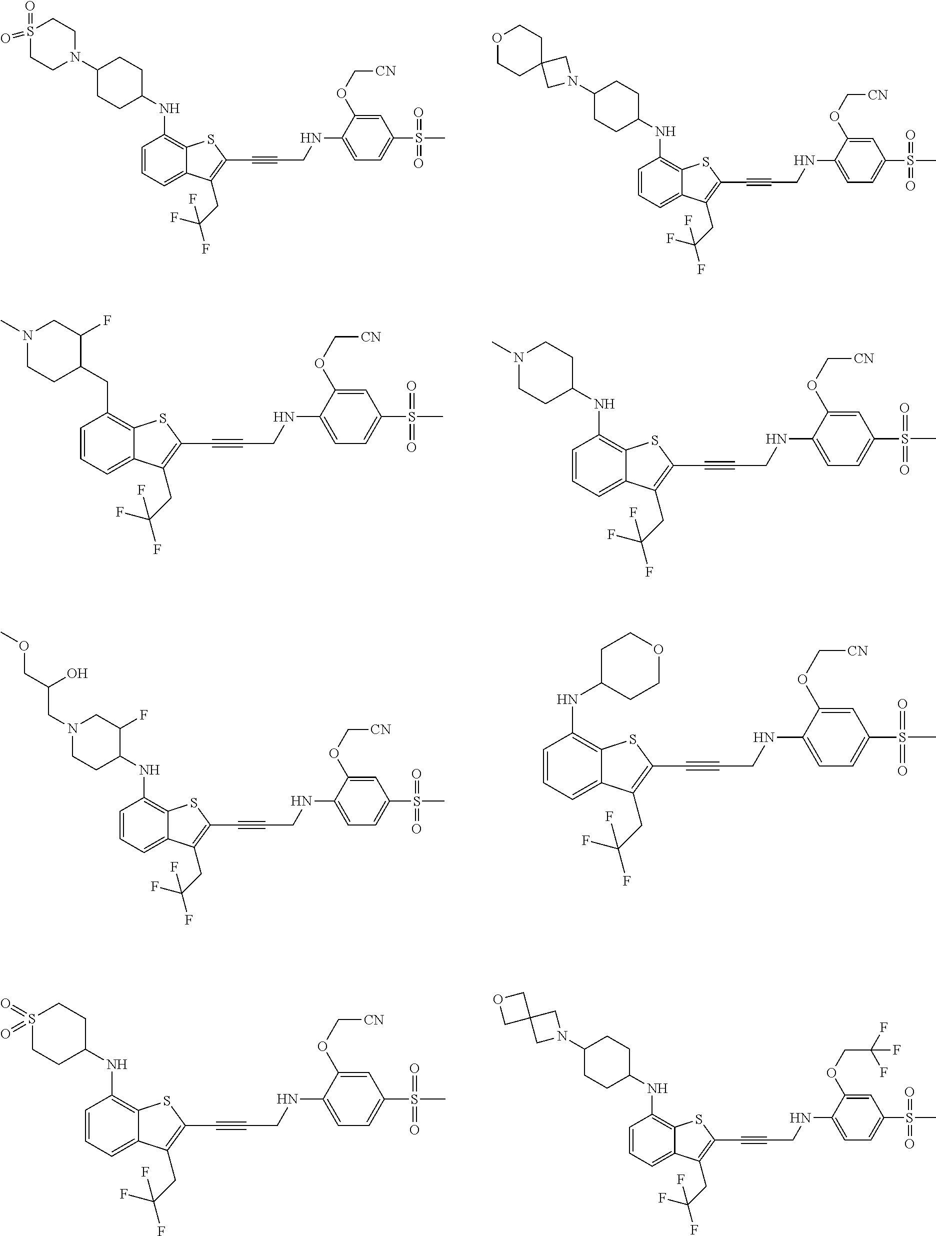

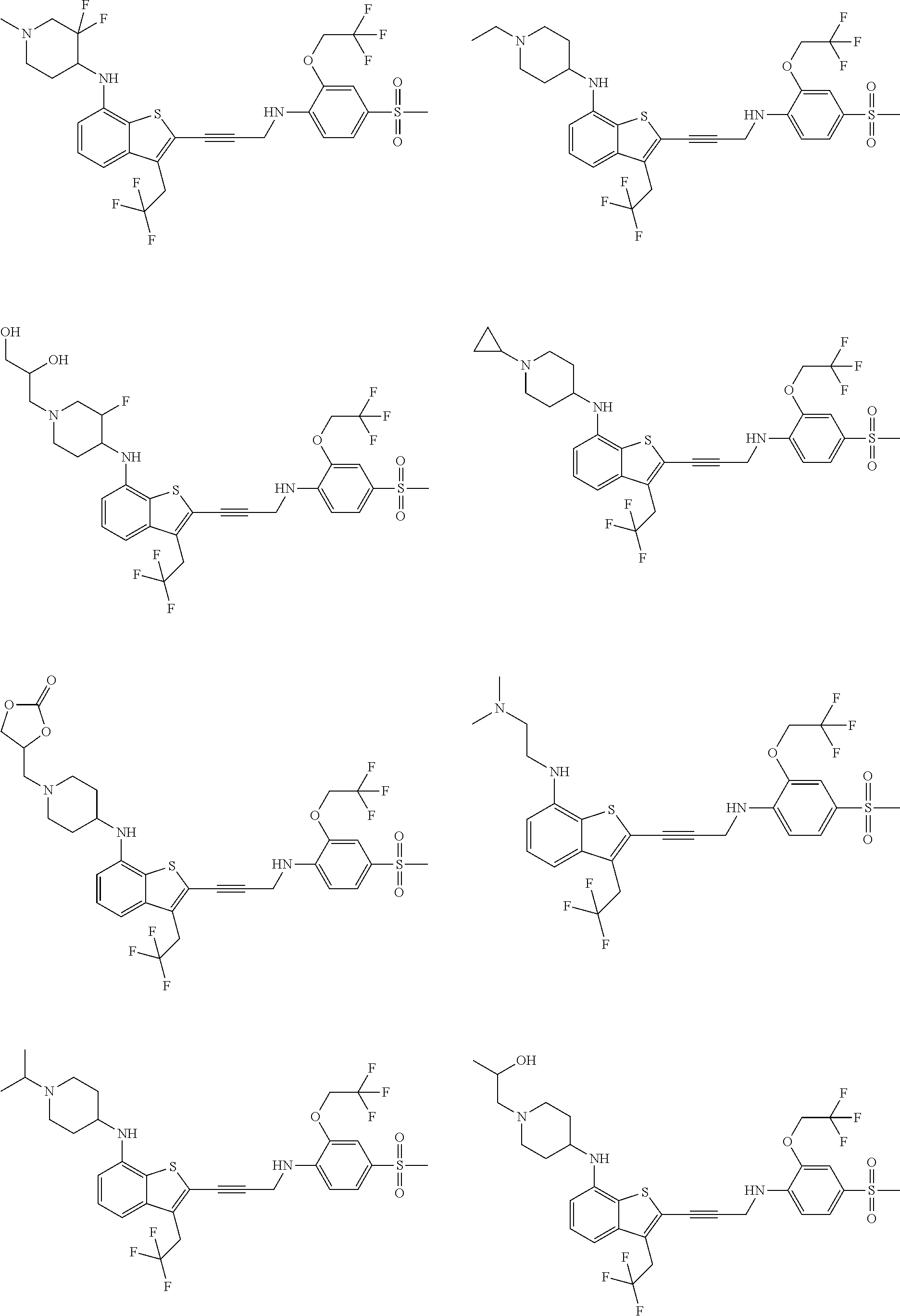

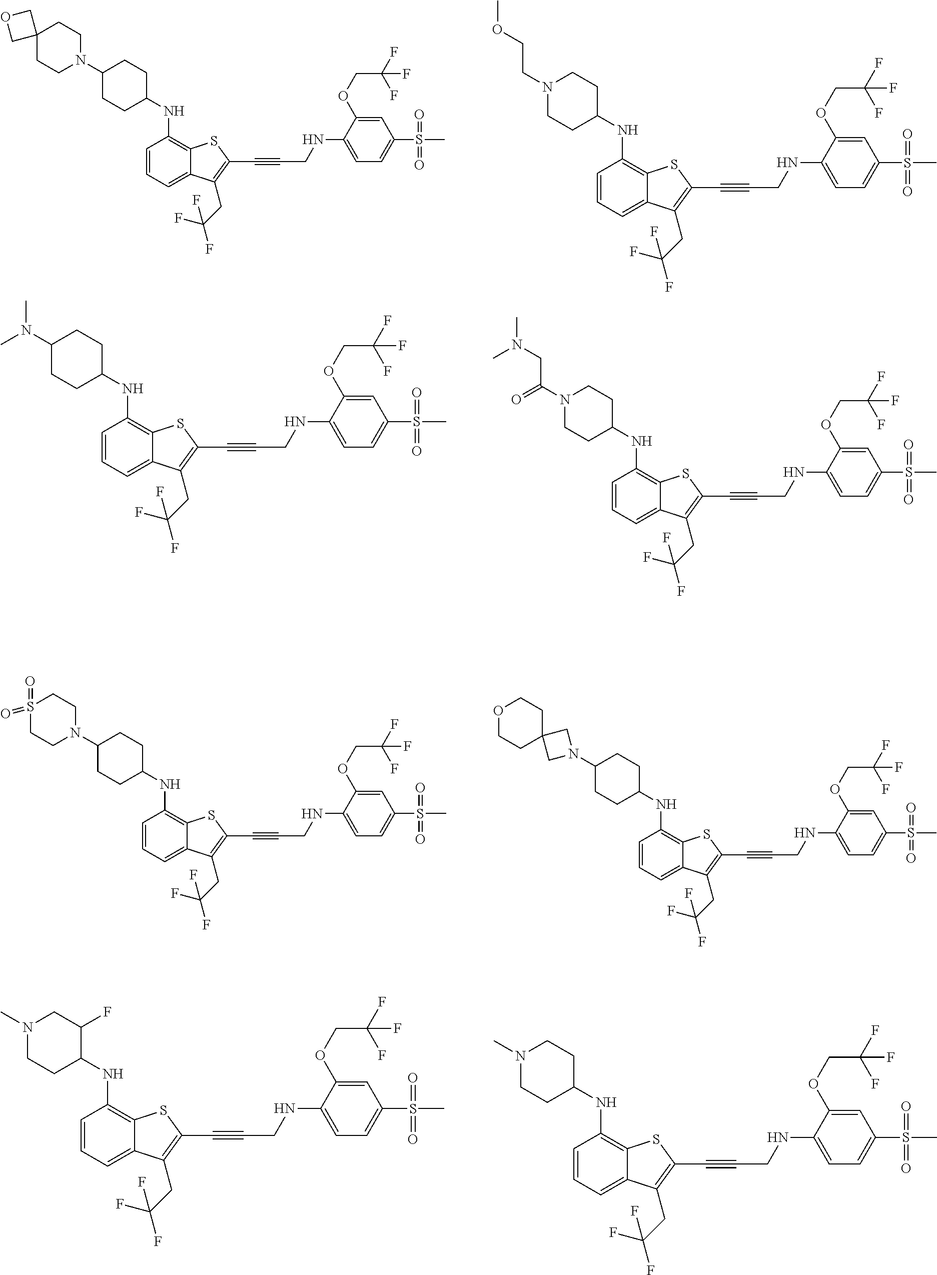

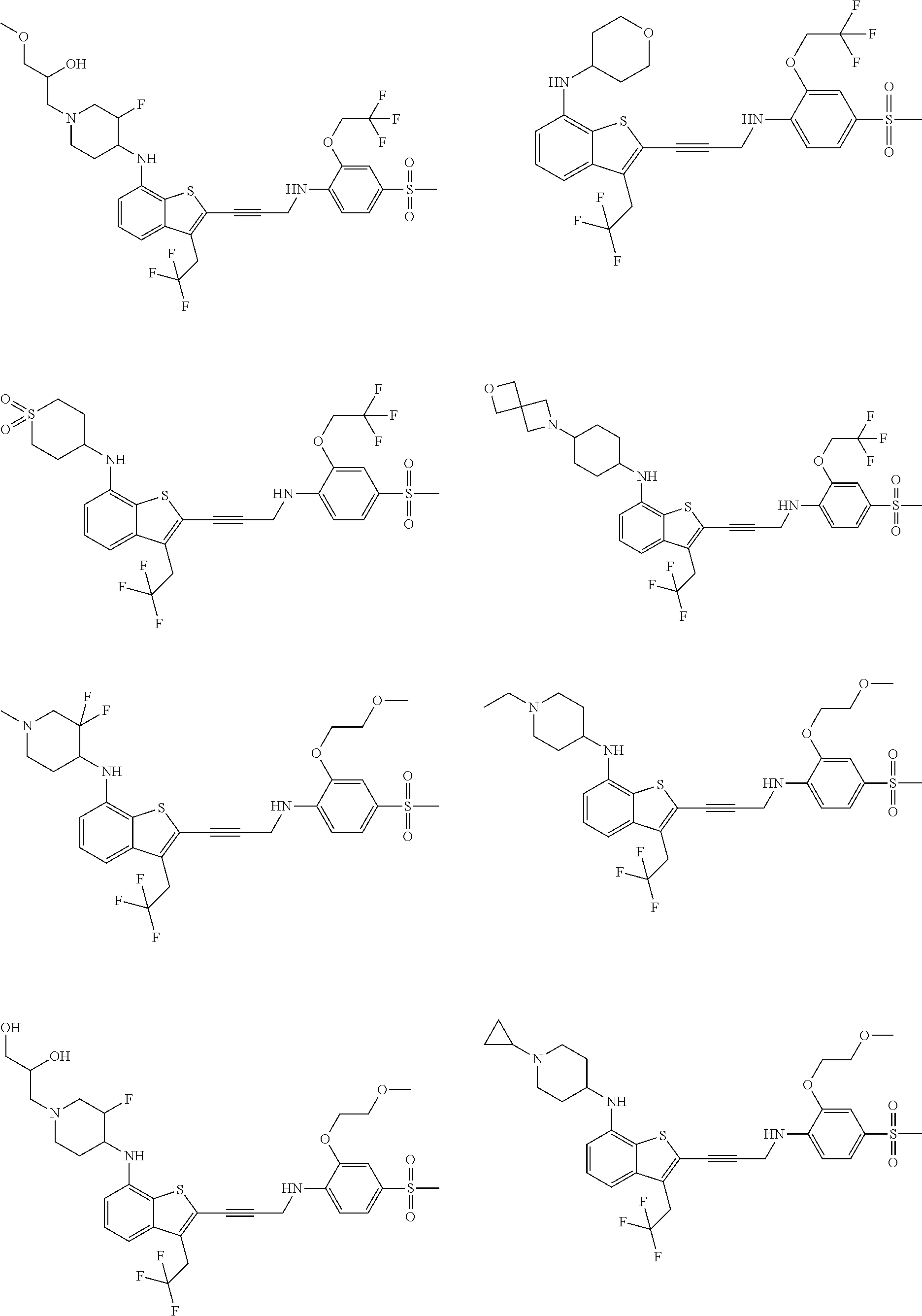

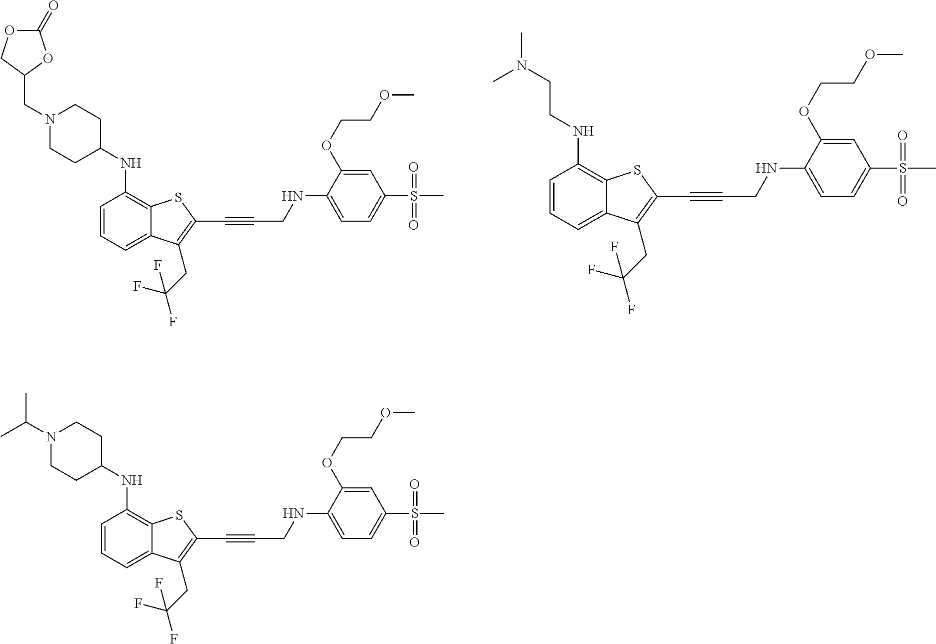

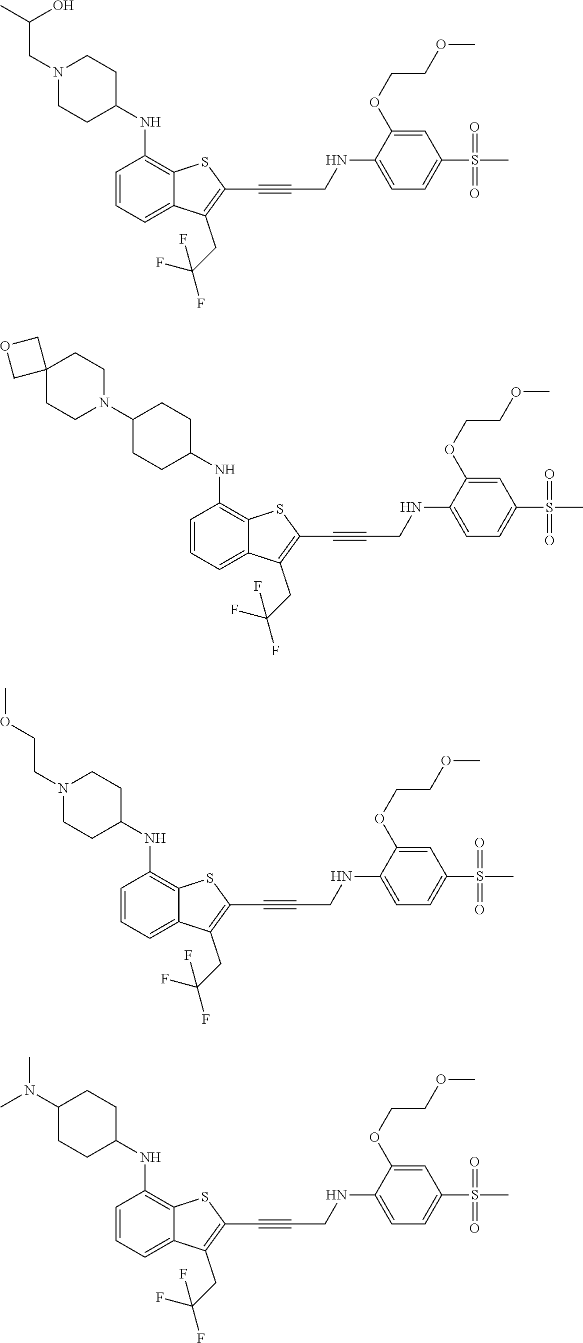

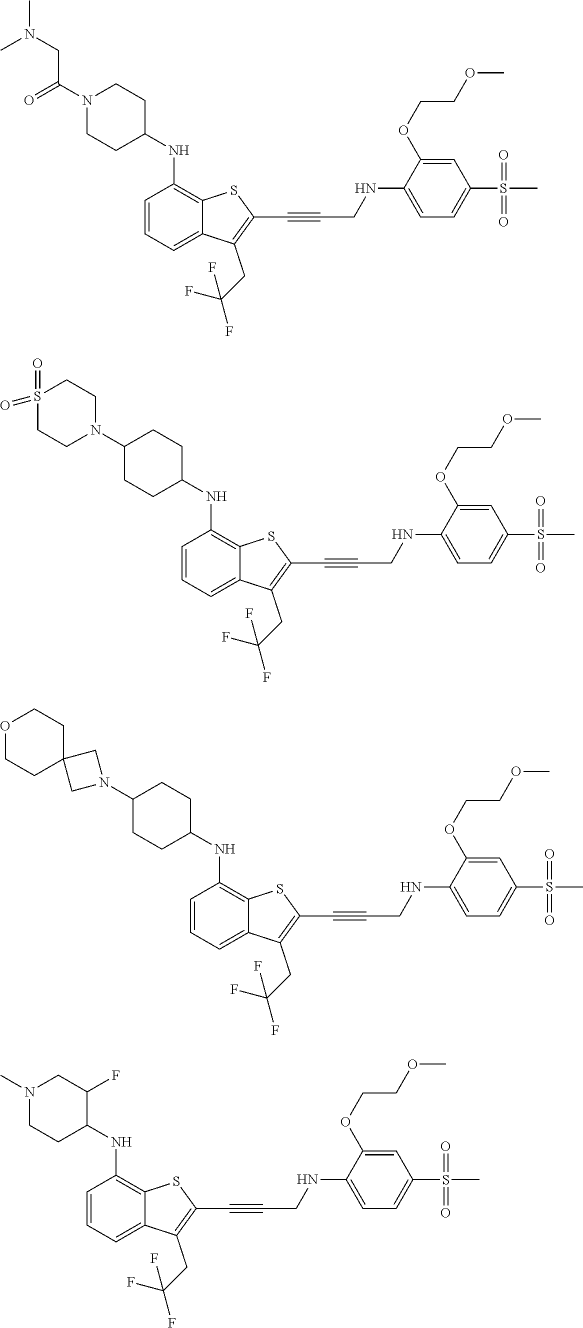

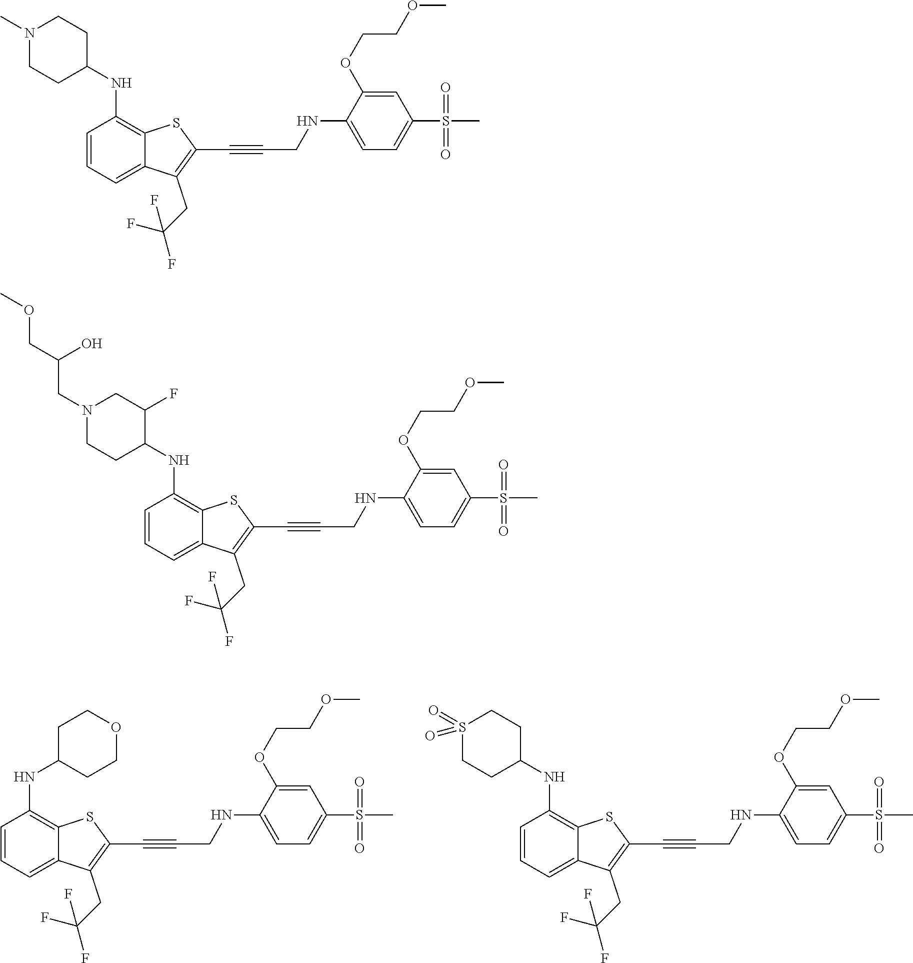

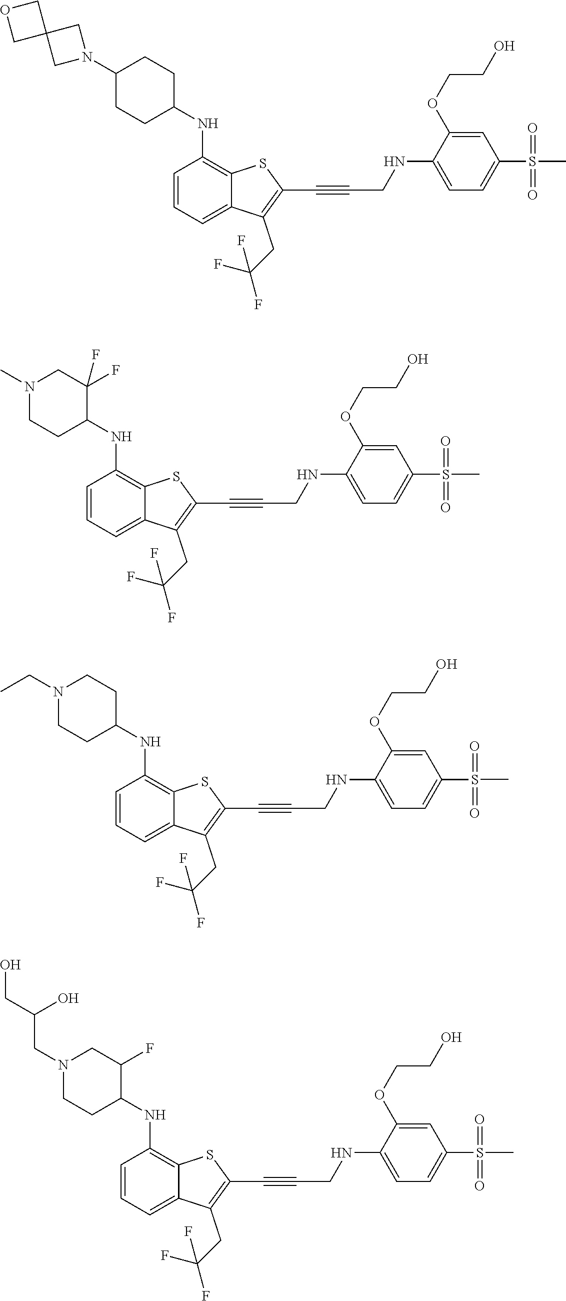

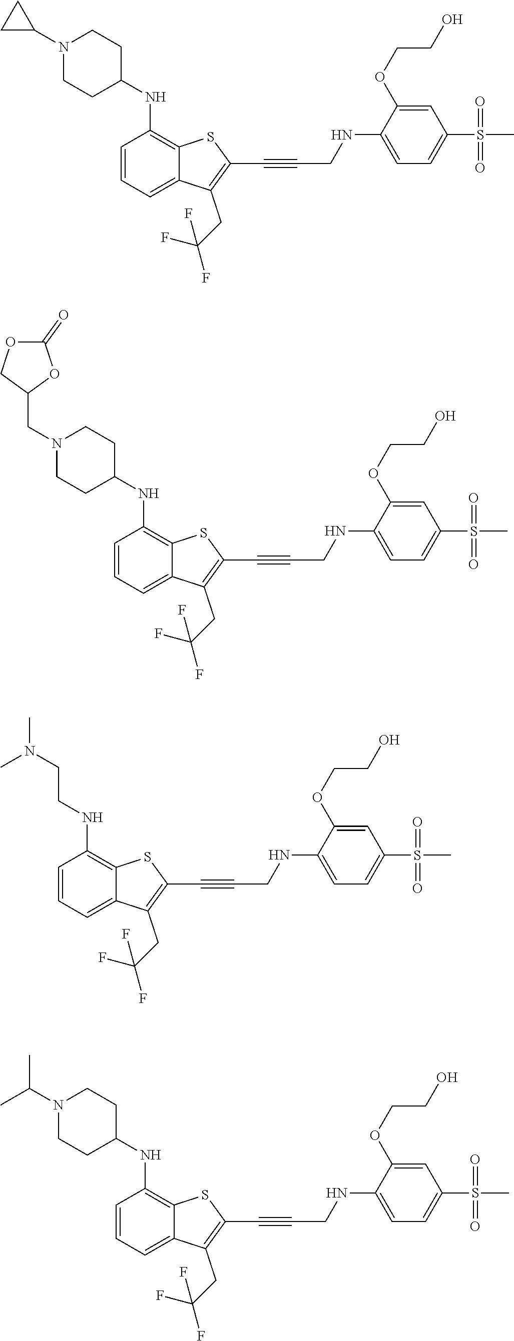

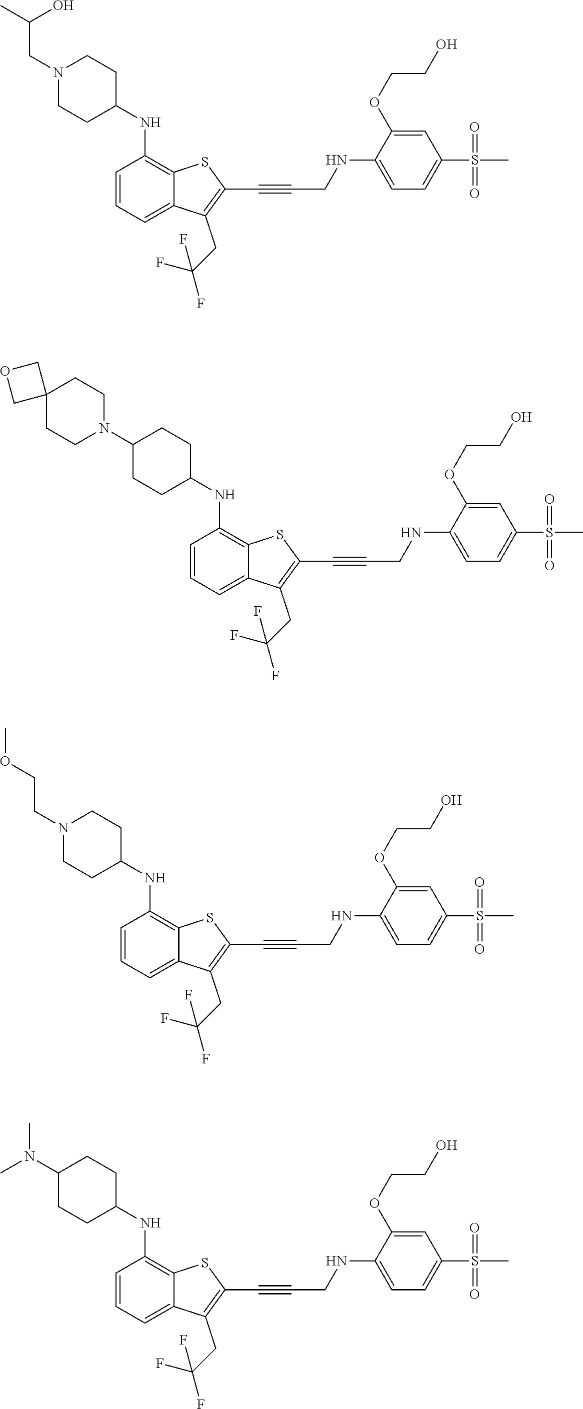

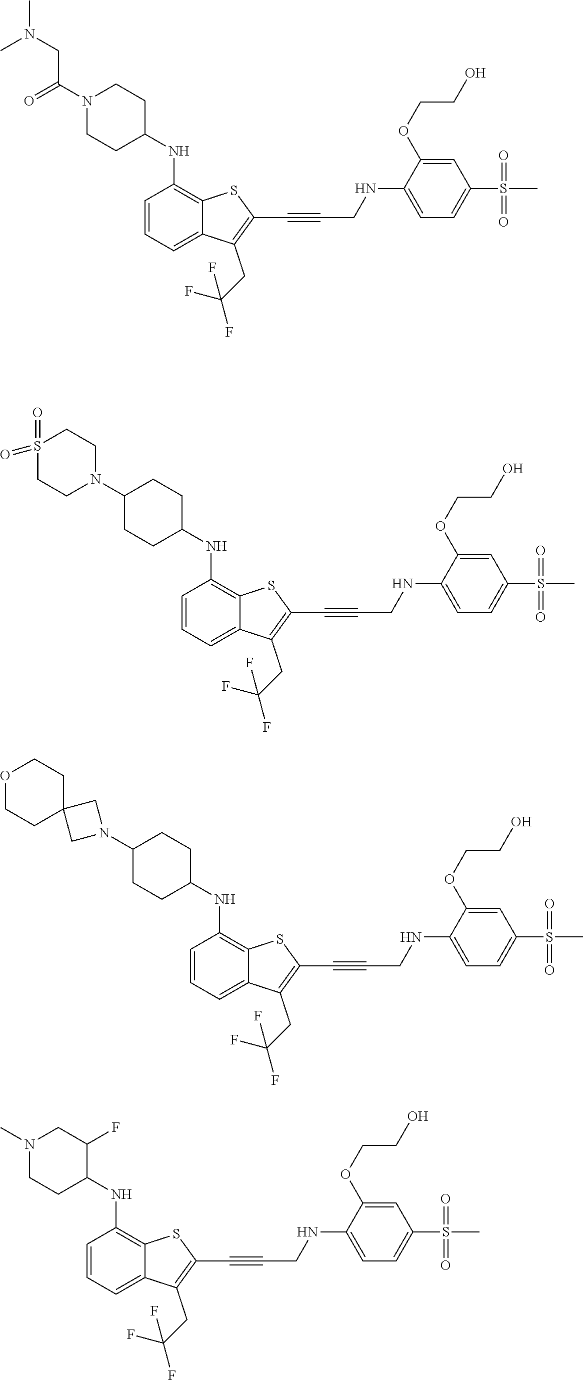

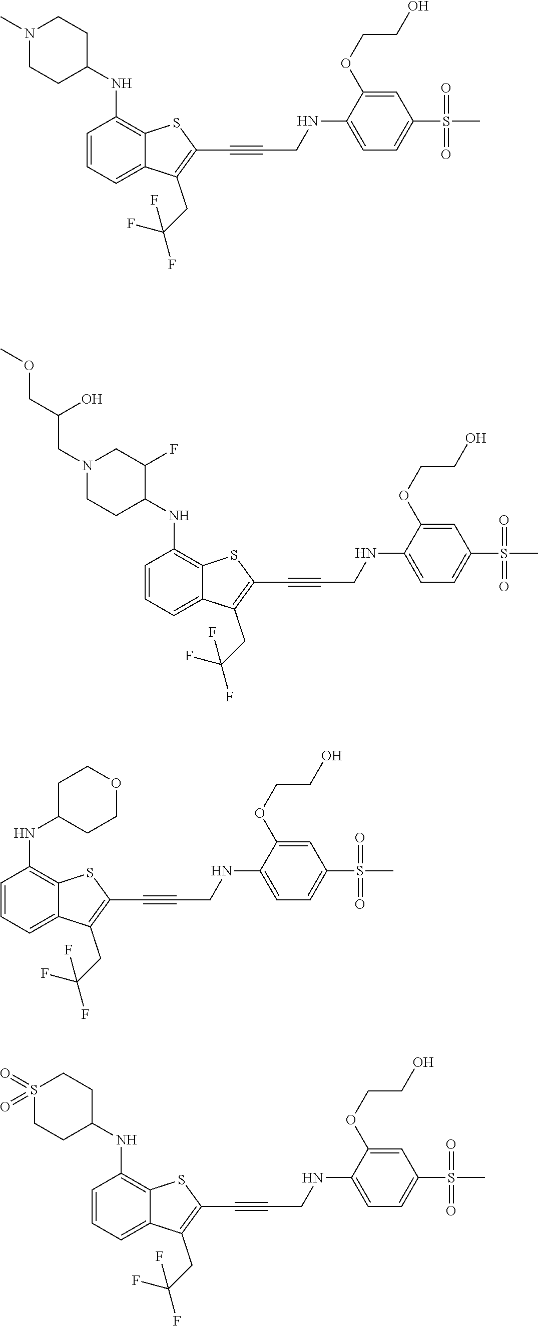

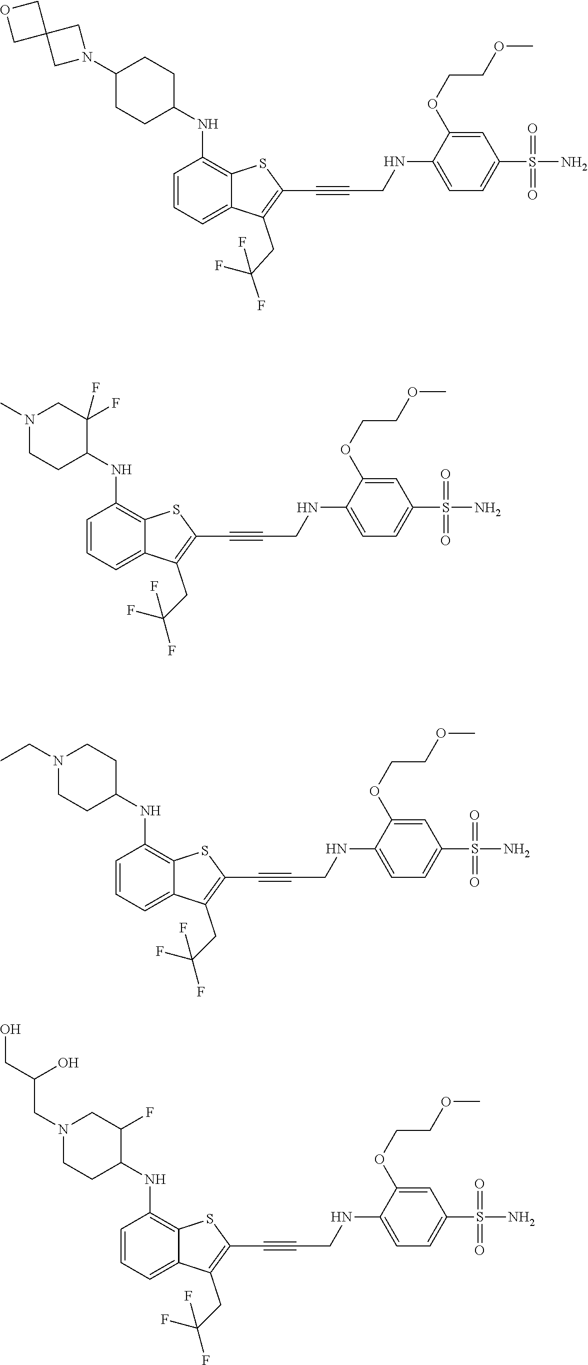 [SMILES: COCCOc1cc(S(N)(=O)=O)ccc1NCC#Cc1sc2c(NC3CCC(N4CC5(COC5)C4)CC3)cccc2c1CC(F)(F)F ; COCCOc1cc(S(N)(=O)=O)ccc1NCC#Cc1sc2c(NC3CCN(C)CC3(F)F)cccc2c1CC(F)(F)F ; CCN1CCC(Nc2cccc3c(CC(F)(F)F)c(C#CCNc4ccc(S(N)(=O)=O)cc4OCCOC)sc23)CC1 ; COCCOc1cc(S(N)(=O)=O)ccc1NCC#Cc1sc2c(NC3CCN(CC(O)CO)CC3F)cccc2c1CC(F)(F)F]

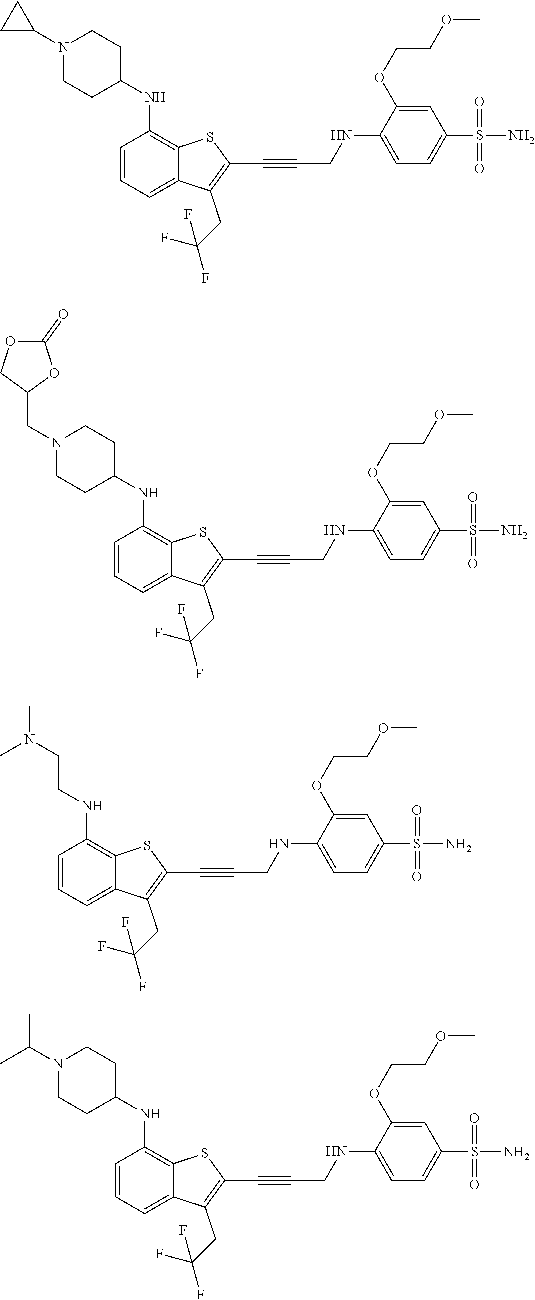 [SMILES: COCCOc1cc(S(N)(=O)=O)ccc1NCC#Cc1sc2c(NC3CCN(C4CC4)CC3)cccc2c1CC(F)(F)F ; COCCOc1cc(S(N)(=O)=O)ccc1NCC#Cc1sc2c(NC3CCN(CC4COC(=O)O4)CC3)cccc2c1CC(F)(F)F ; COCCOc1cc(S(N)(=O)=O)ccc1NCC#Cc1sc2c(NCCN(C)C)cccc2c1CC(F)(F)F ; COCCOc1cc(S(N)(=O)=O)ccc1NCC#Cc1sc2c(NC3CCN(C(C)C)CC3)cccc2c1CC(F)(F)F]

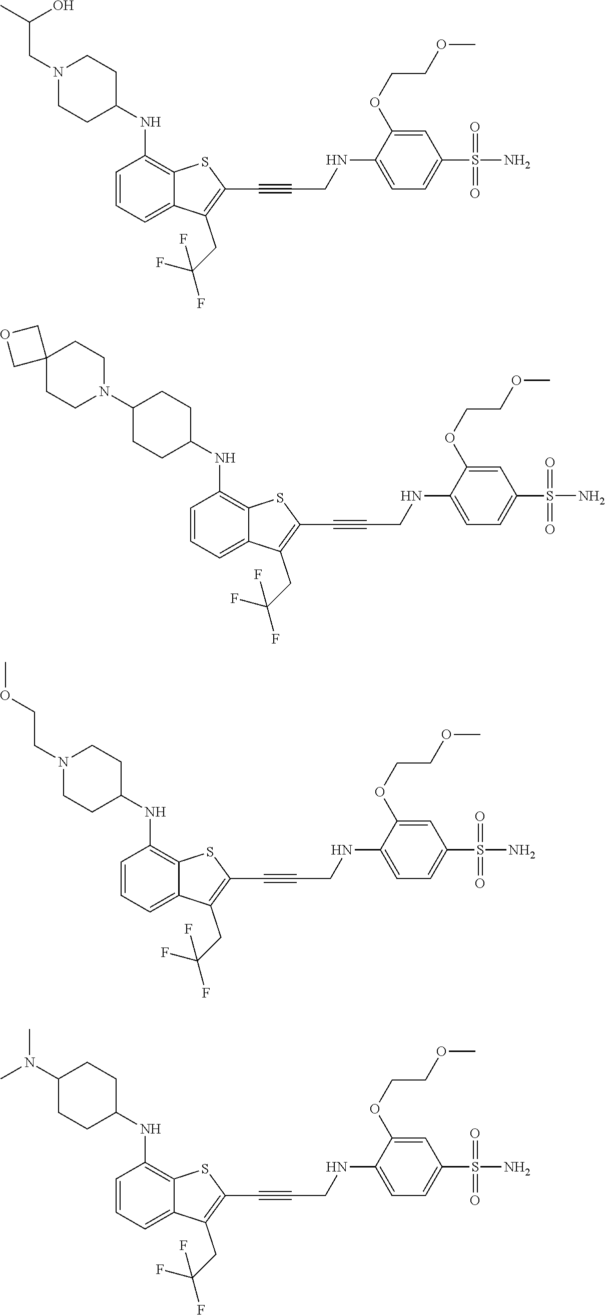 [SMILES: COCCOc1cc(S(N)(=O)=O)ccc1NCC#Cc1sc2c(NC3CCN(CC(C)O)CC3)cccc2c1CC(F)(F)F ; COCCOc1cc(S(N)(=O)=O)ccc1NCC#Cc1sc2c(NC3CCC(N4CCC5(CC4)COC5)CC3)cccc2c1CC(F)(F)F ; COCCOc1cc(S(N)(=O)=O)ccc1NCC#Cc1sc2c(NC3CCN(CCOC)CC3)cccc2c1CC(F)(F)F ; COCCOc1cc(S(N)(=O)=O)ccc1NCC#Cc1sc2c(NC3CCC(N(C)C)CC3)cccc2c1CC(F)(F)F]

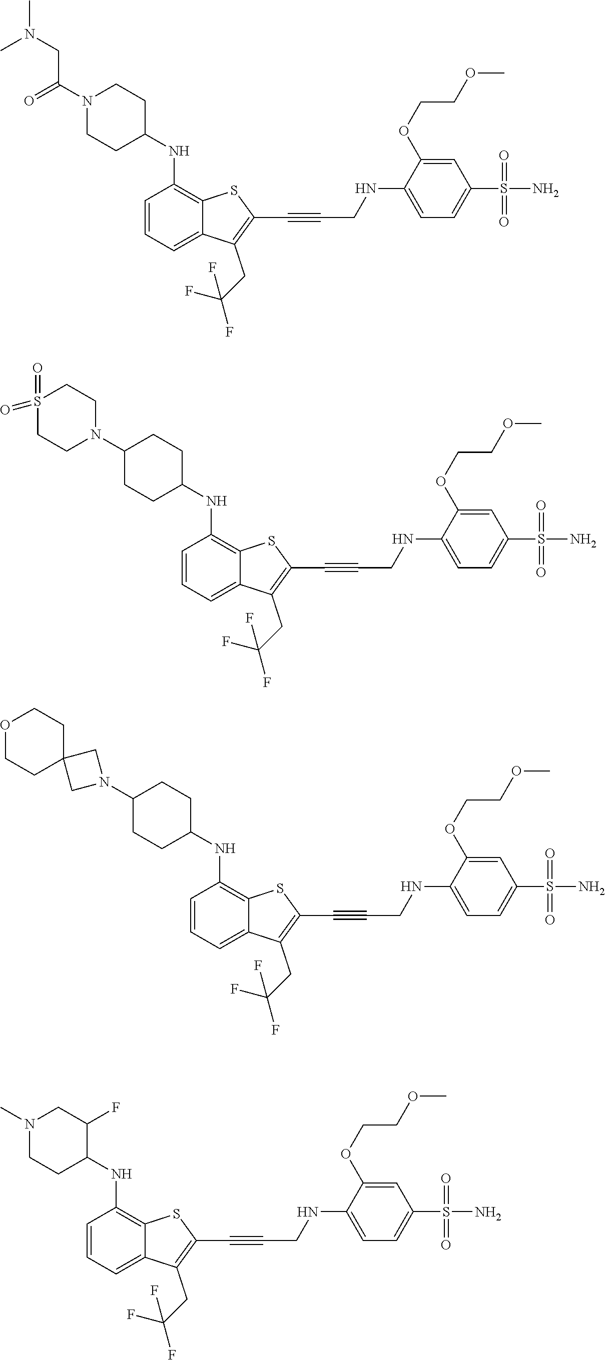 [SMILES: COCCOc1cc(S(N)(=O)=O)ccc1NCC#Cc1sc2c(NC3CCN(C(=O)CN(C)C)CC3)cccc2c1CC(F)(F)F ; COCCOc1cc(S(N)(=O)=O)ccc1NCC#Cc1sc2c(NC3CCC(N4CCS(=O)(=O)CC4)CC3)cccc2c1CC(F)(F)F ; COCCOc1cc(S(N)(=O)=O)ccc1NCC#Cc1sc2c(NC3CCC(N4CC5(CCOCC5)C4)CC3)cccc2c1CC(F)(F)F ; COCCOc1cc(S(N)(=O)=O)ccc1NCC#Cc1sc2c(NC3CCN(C)CC3F)cccc2c1CC(F)(F)F]

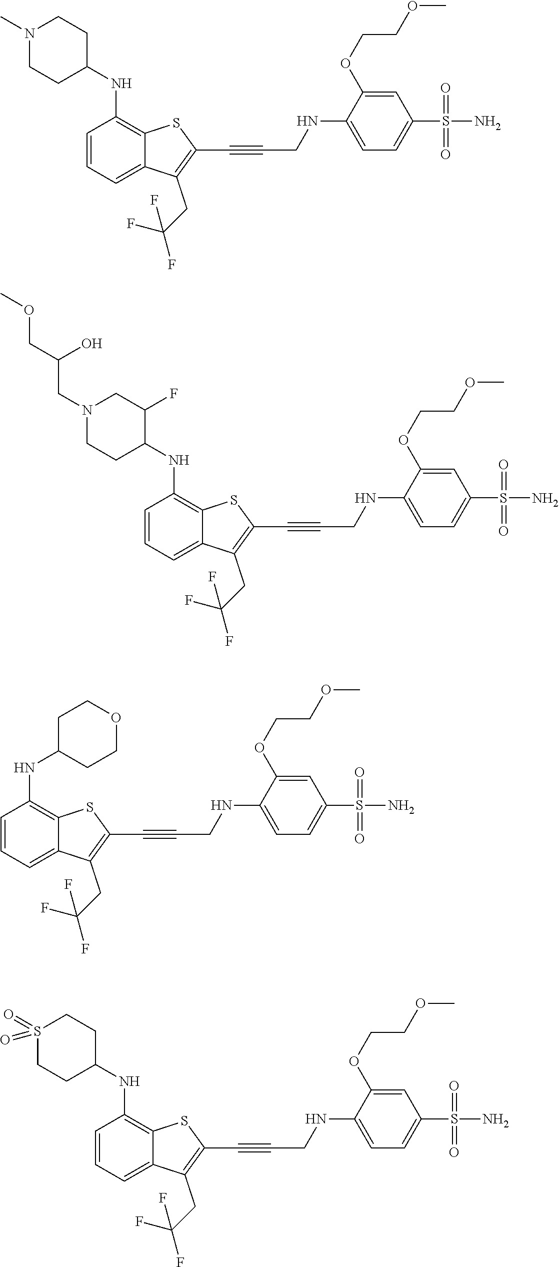 [SMILES: COCCOc1cc(S(N)(=O)=O)ccc1NCC#Cc1sc2c(NC3CCN(C)CC3)cccc2c1CC(F)(F)F ; COCCOc1cc(S(N)(=O)=O)ccc1NCC#Cc1sc2c(NC3CCN(CC(O)COC)CC3F)cccc2c1CC(F)(F)F ; COCCOc1cc(S(N)(=O)=O)ccc1NCC#Cc1sc2c(NC3CCOCC3)cccc2c1CC(F)(F)F ; COCCOc1cc(S(N)(=O)=O)ccc1NCC#Cc1sc2c(NC3CCS(=O)(=O)CC3)cccc2c1CC(F)(F)F]

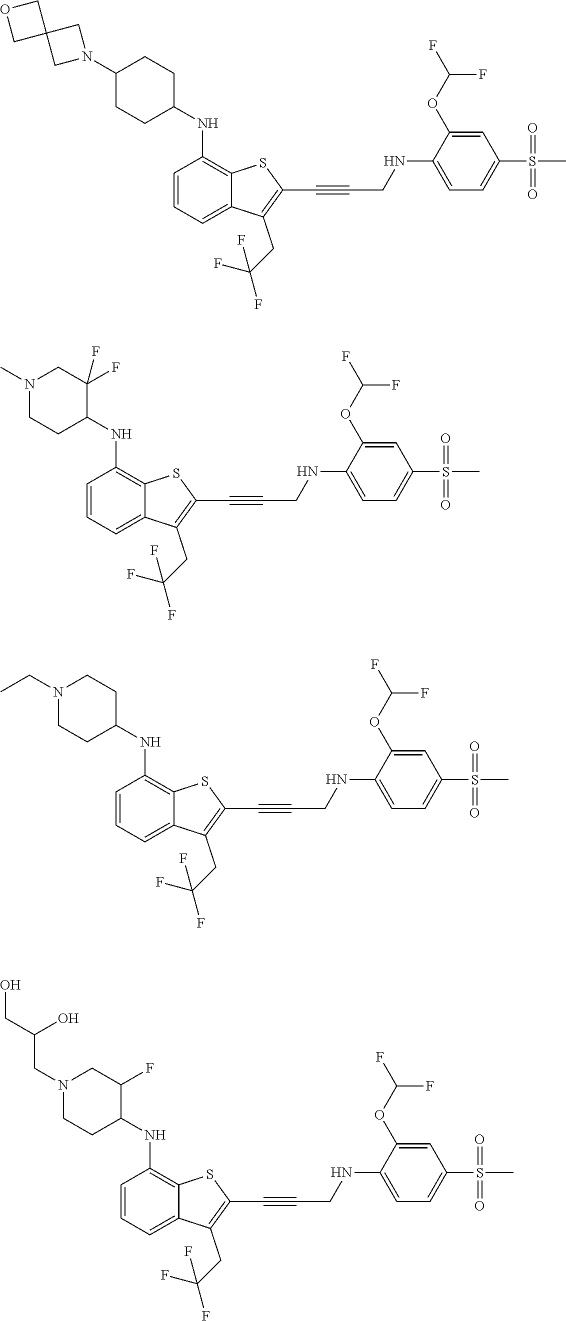

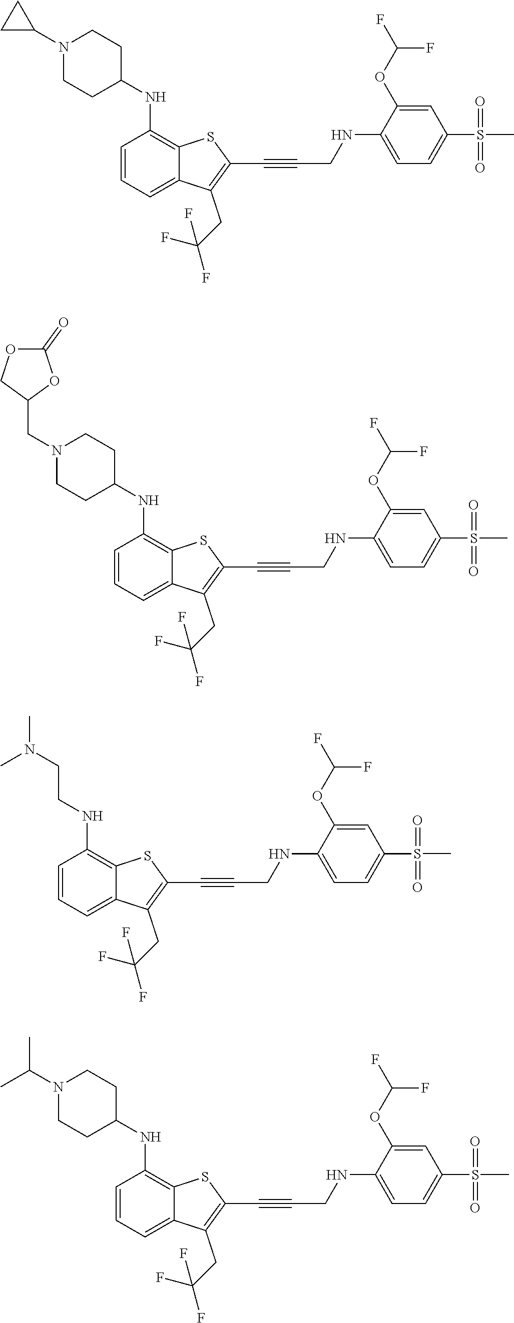

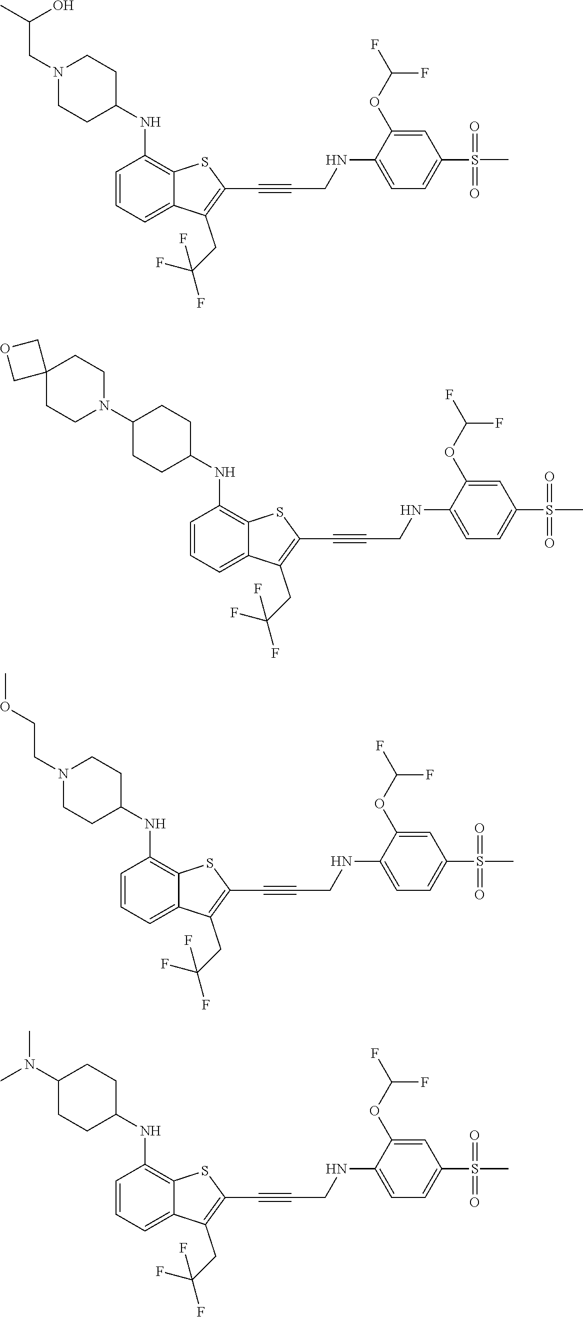

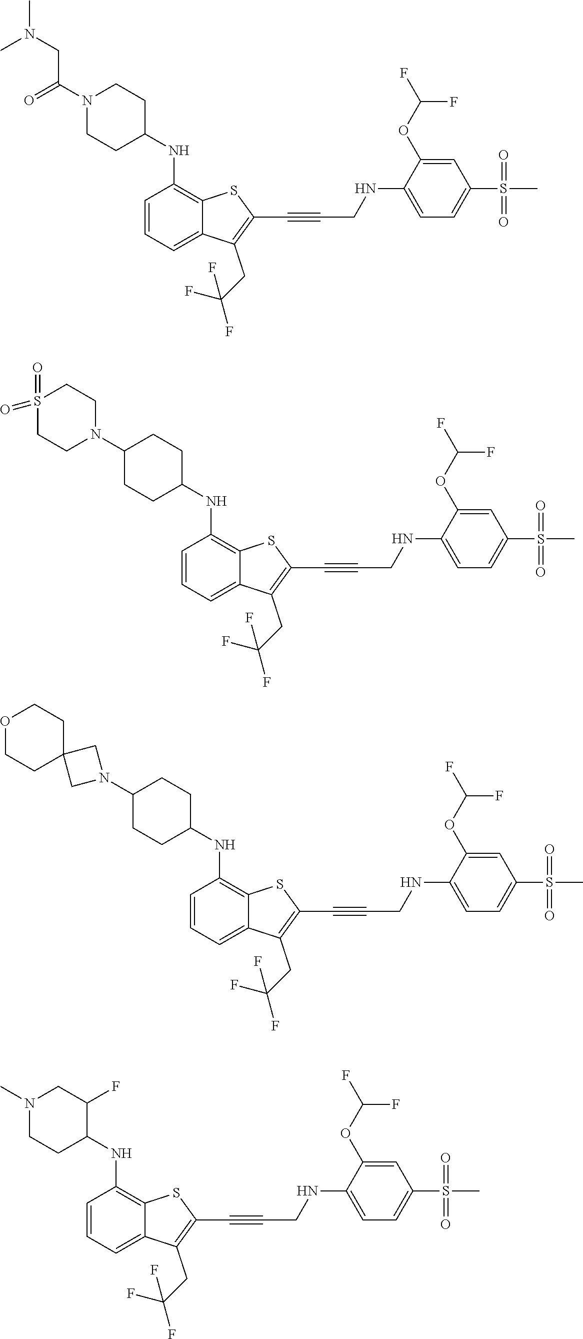

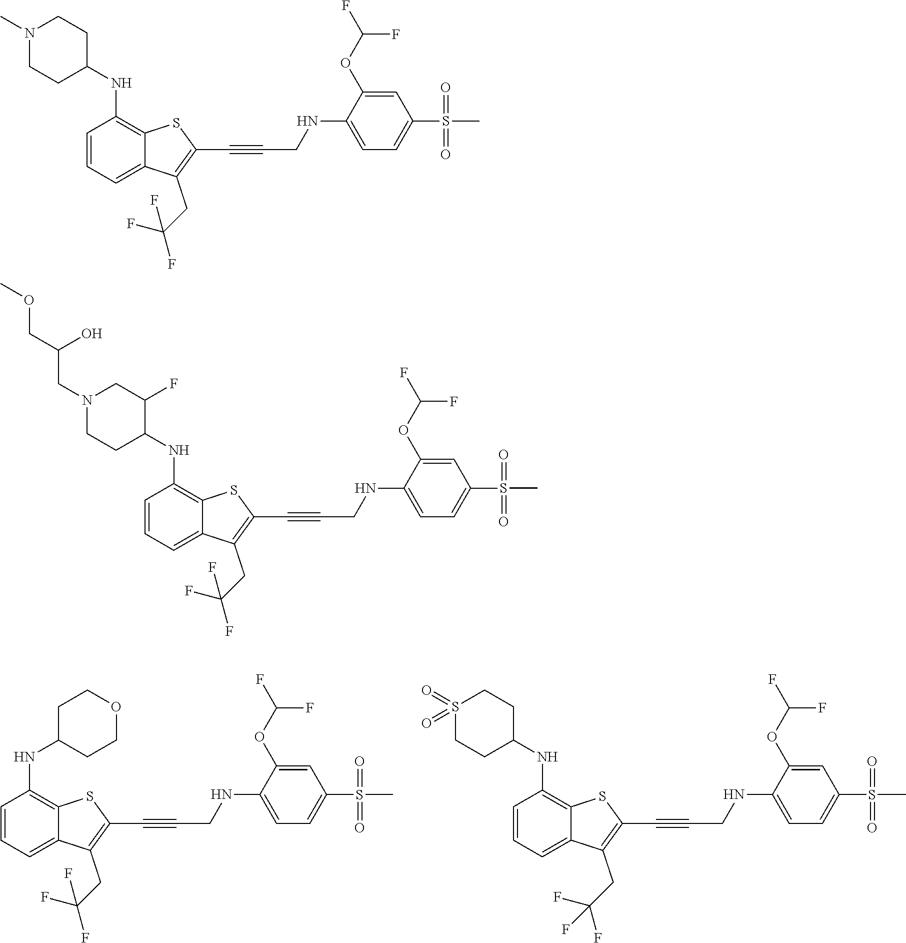

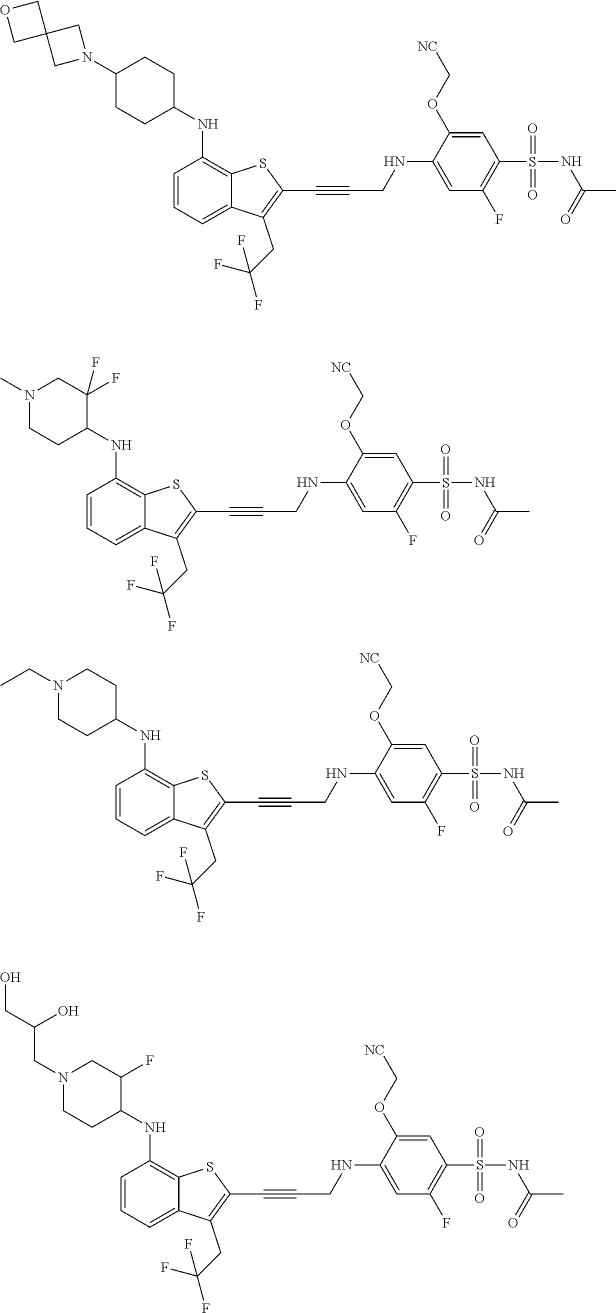

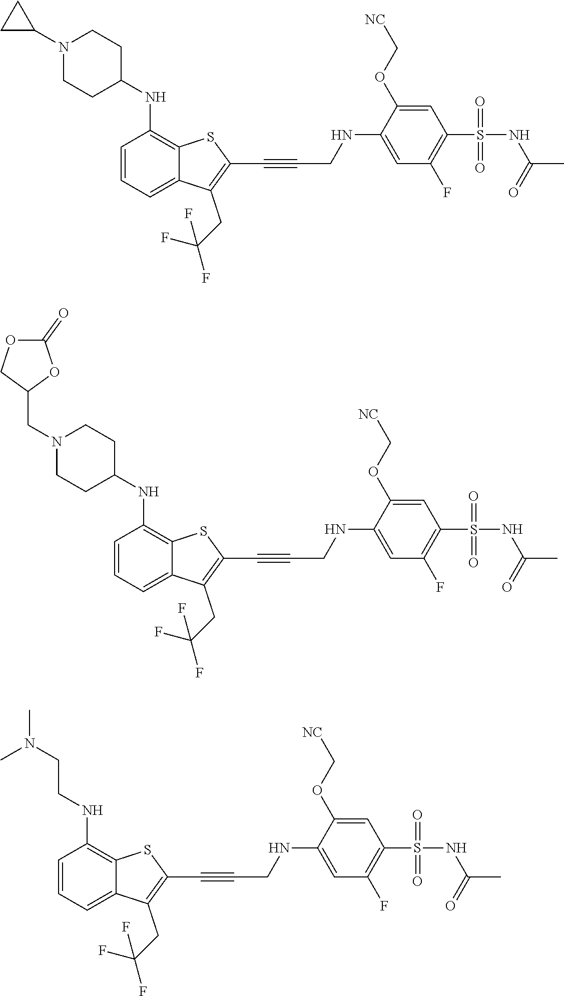

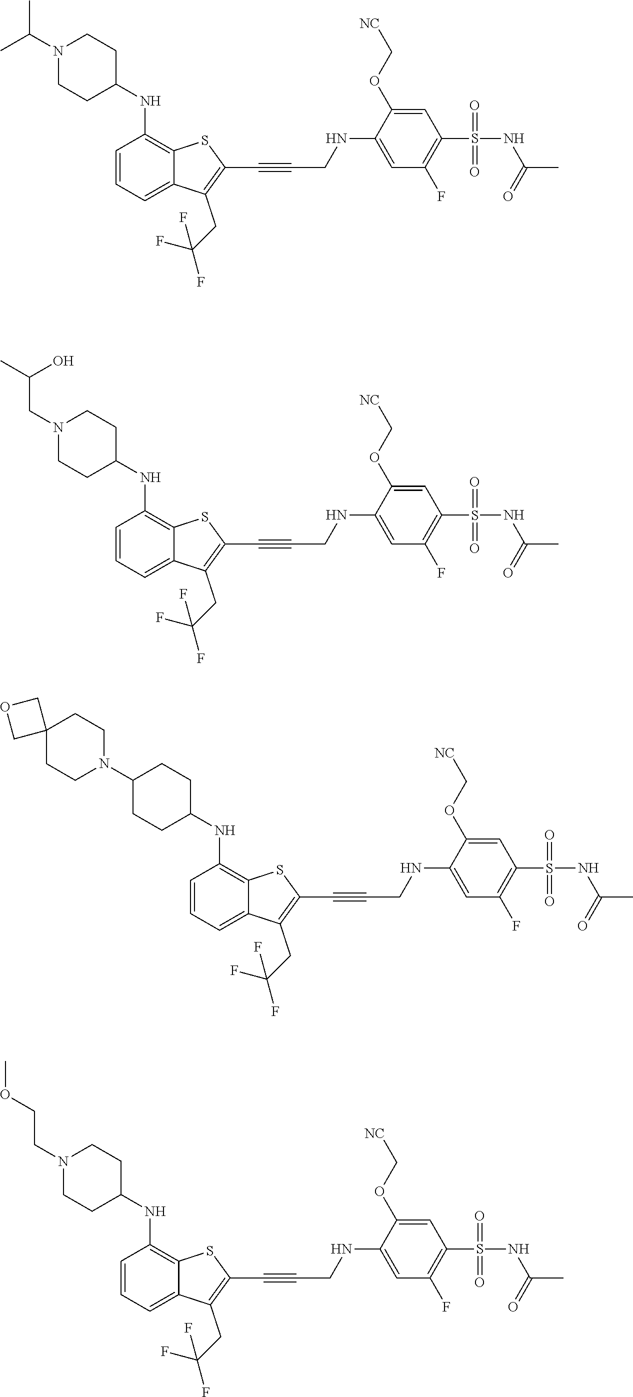

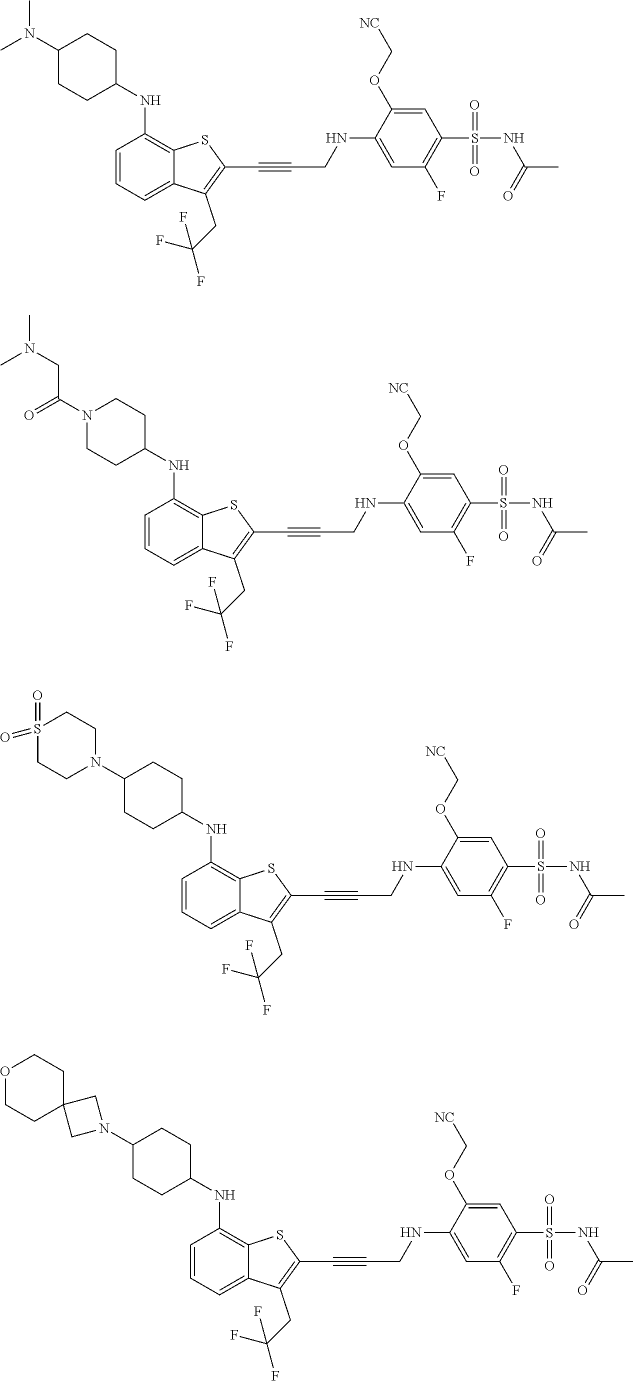

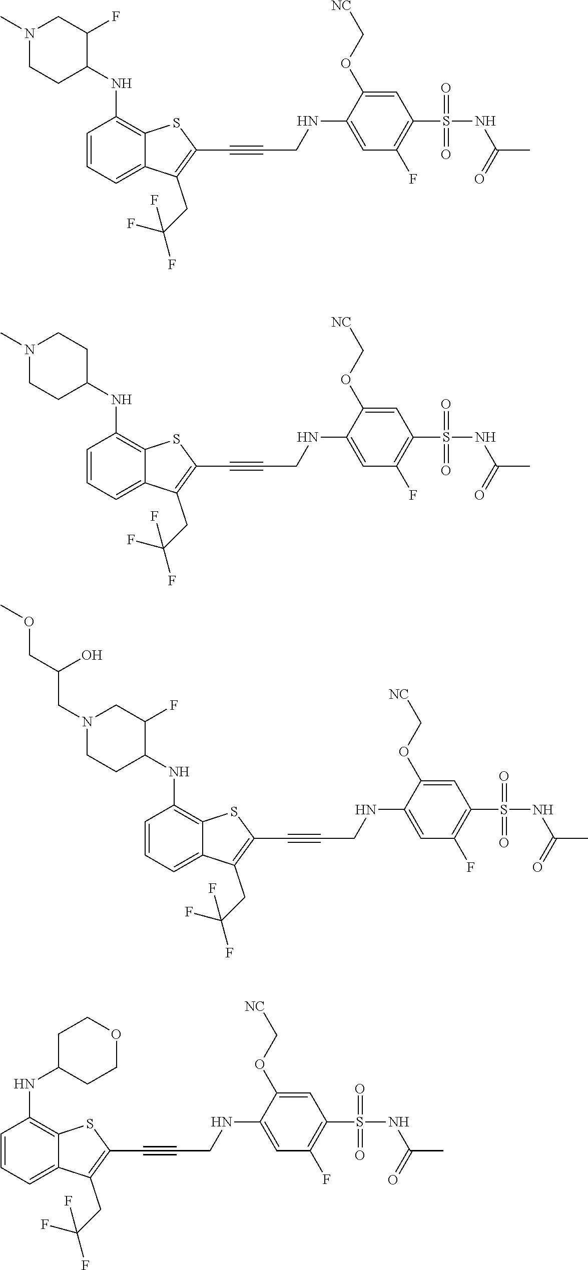

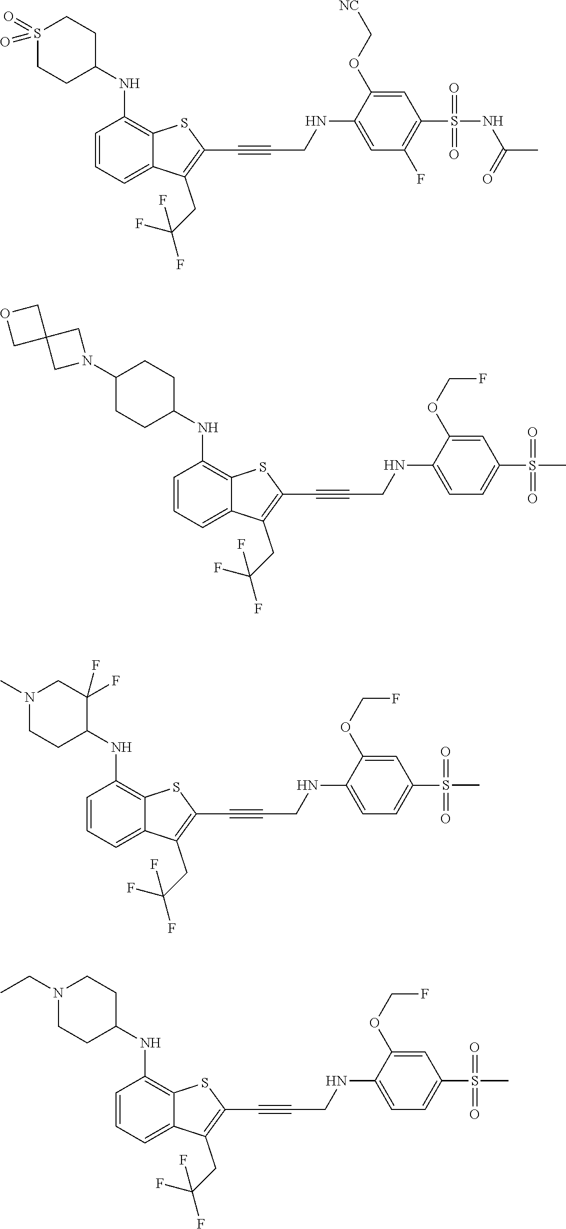

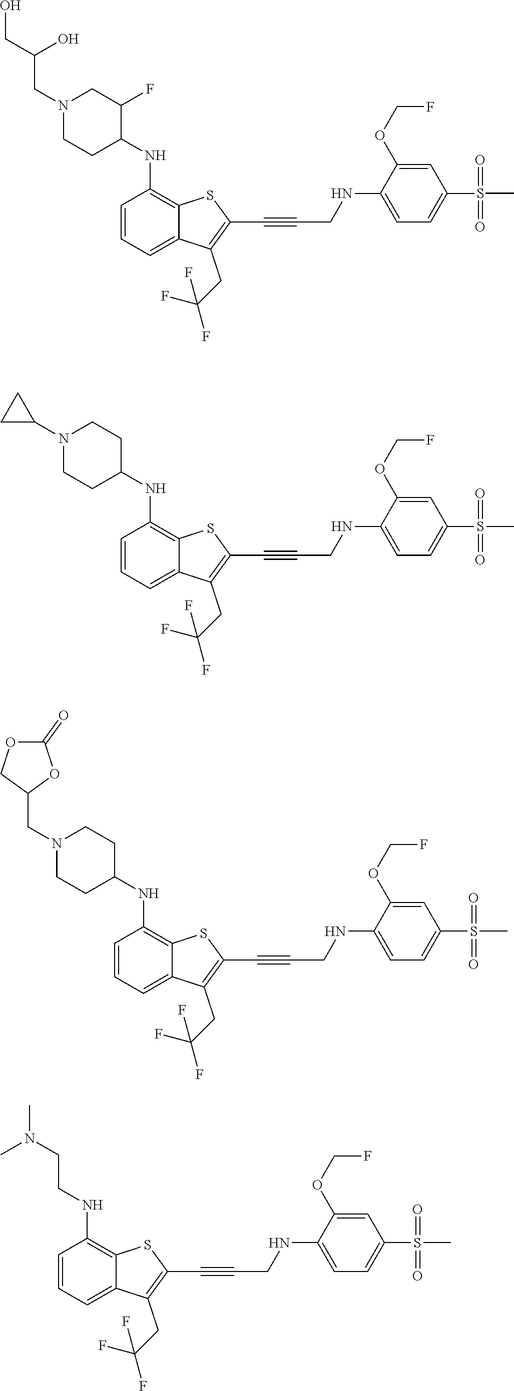

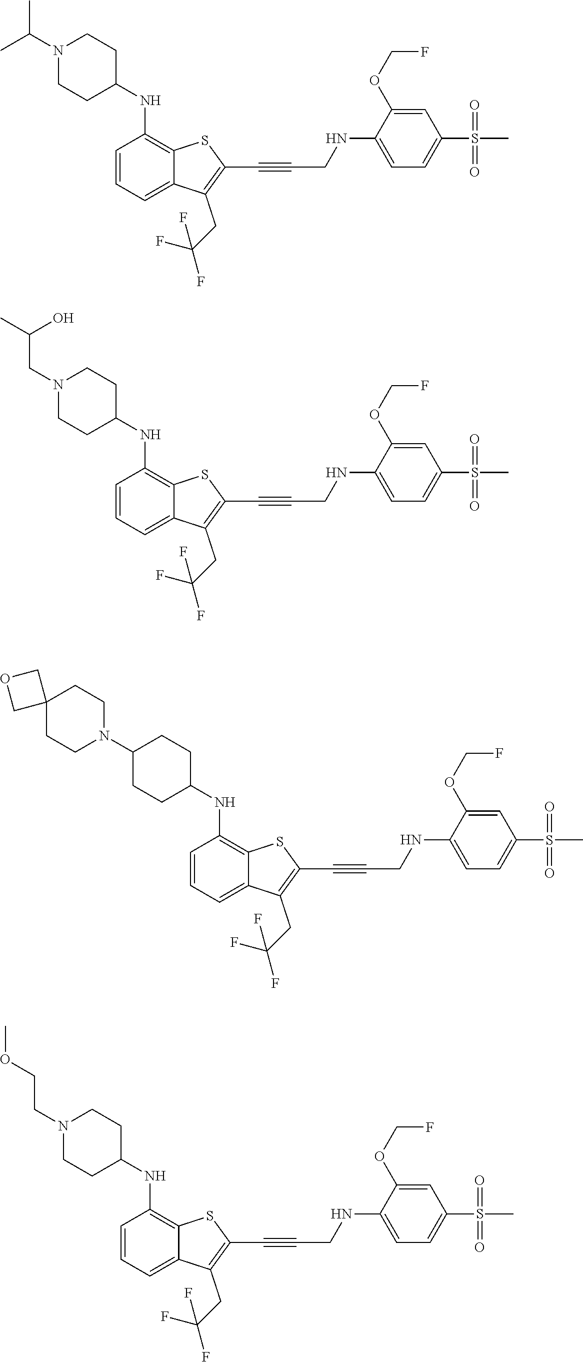

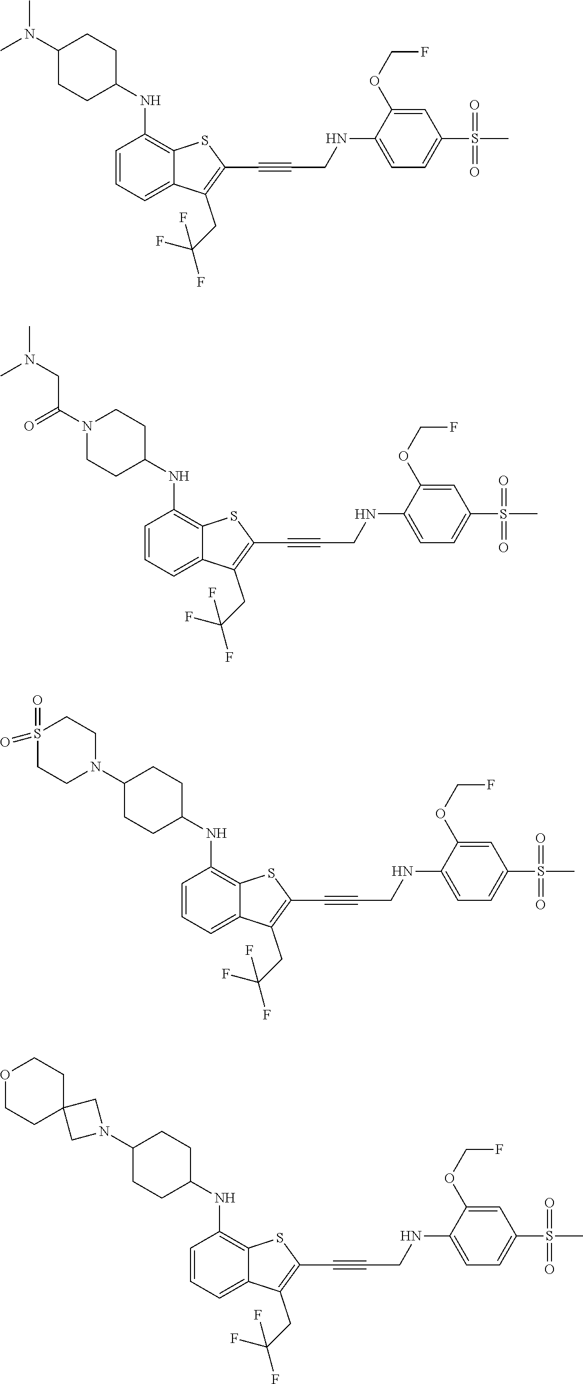

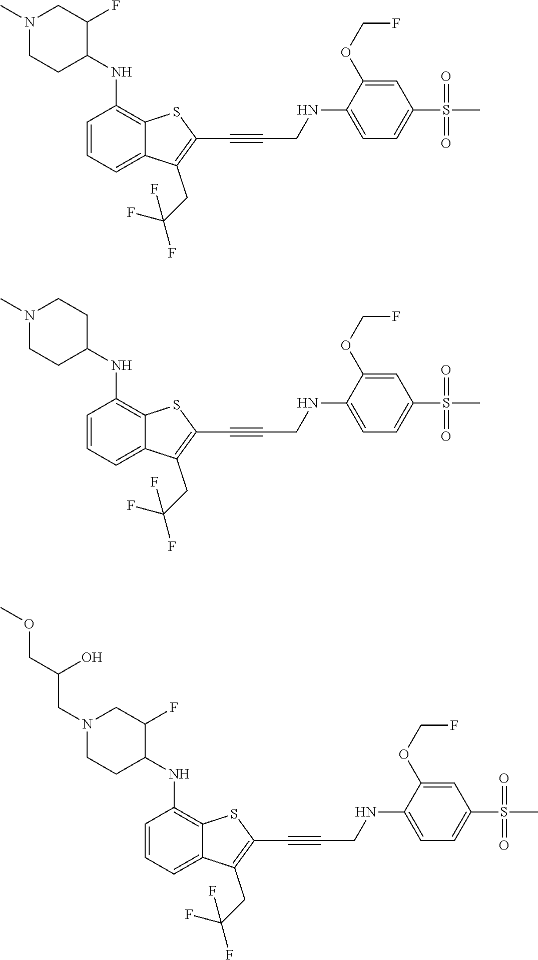

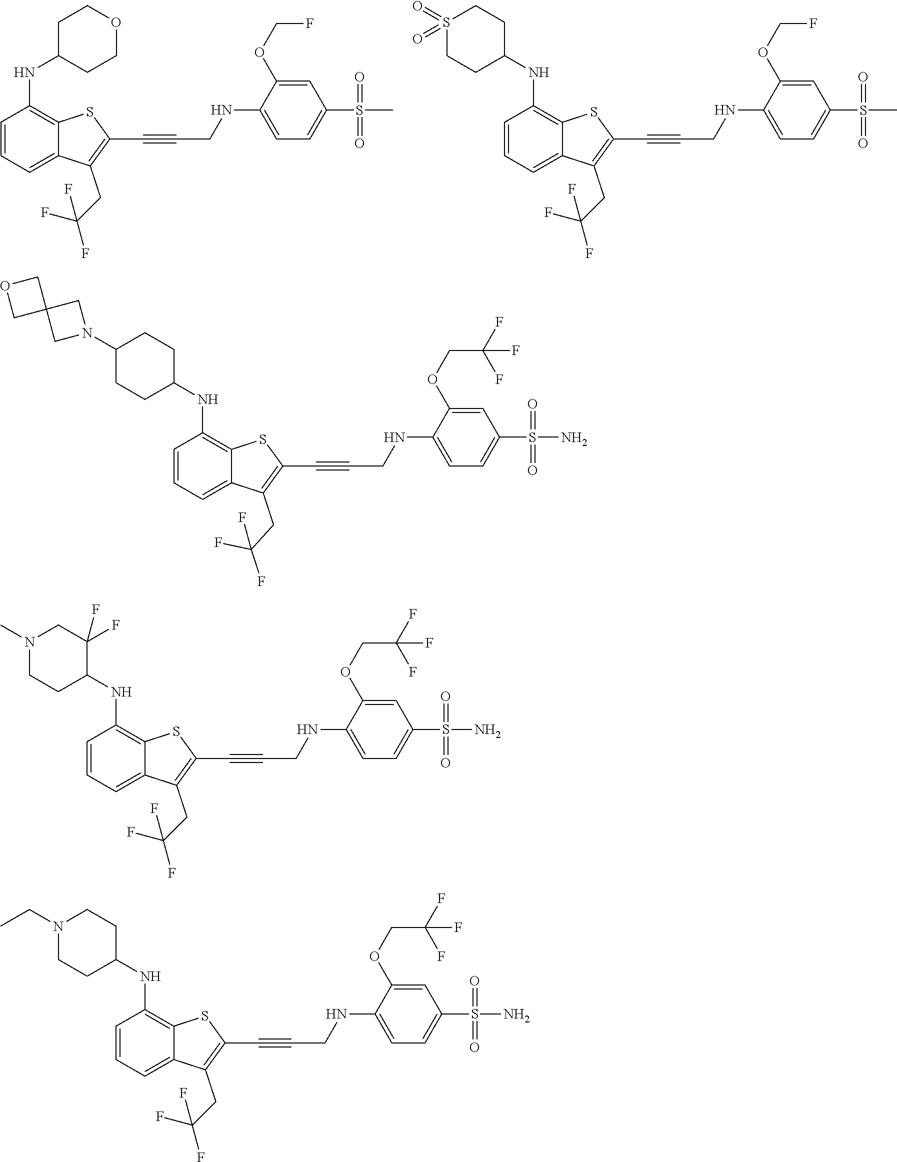 [SMILES: CS(=O)(=O)c1ccc(NCC#Cc2sc3c(NC4CCOCC4)cccc3c2CC(F)(F)F)c(OCF)c1 ; CS(=O)(=O)c1ccc(NCC#Cc2sc3c(NC4CCS(=O)(=O)CC4)cccc3c2CC(F)(F)F)c(OCF)c1 ; NS(=O)(=O)c1ccc(NCC#Cc2sc3c(NC4CCC(N5CC6(COC6)C5)CC4)cccc3c2CC(F)(F)F)c(OCC(F)(F)F)c1 ; CN1CCC(Nc2cccc3c(CC(F)(F)F)c(C#CCNc4ccc(S(N)(=O)=O)cc4OCC(F)(F)F)sc23)C(F)(F)C1 ; CCN1CCC(Nc2cccc3c(CC(F)(F)F)c(C#CCNc4ccc(S(N)(=O)=O)cc4OCC(F)(F)F)sc23)CC1]

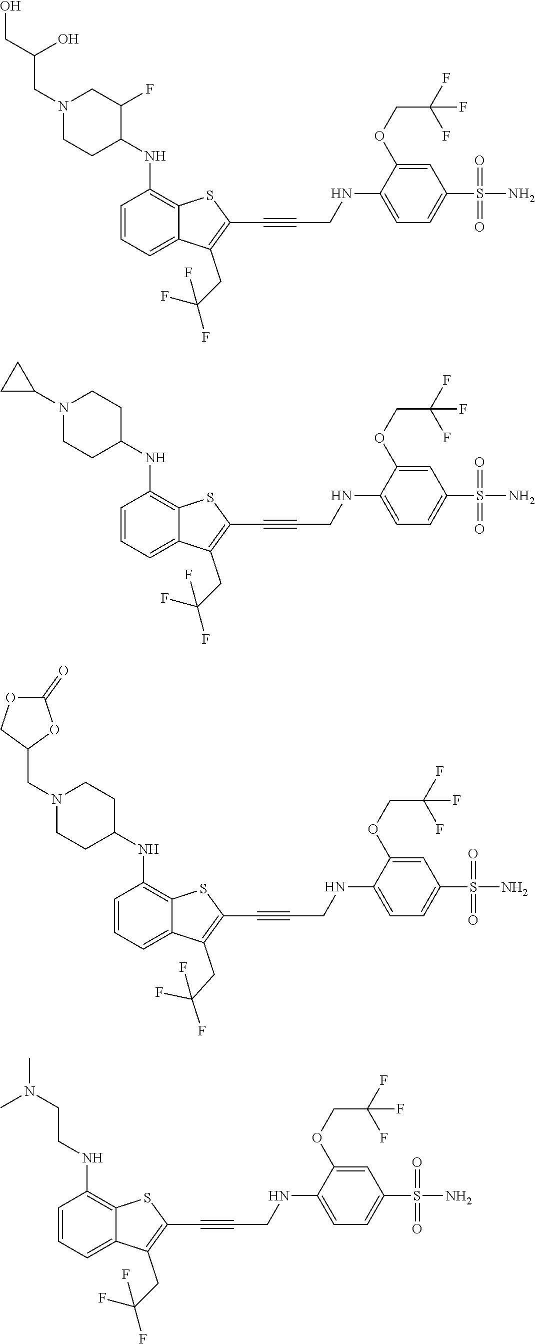 [SMILES: NS(=O)(=O)c1ccc(NCC#Cc2sc3c(NC4CCN(CC(O)CO)CC4F)cccc3c2CC(F)(F)F)c(OCC(F)(F)F)c1 ; NS(=O)(=O)c1ccc(NCC#Cc2sc3c(NC4CCN(C5CC5)CC4)cccc3c2CC(F)(F)F)c(OCC(F)(F)F)c1 ; NS(=O)(=O)c1ccc(NCC#Cc2sc3c(NC4CCN(CC5COC(=O)O5)CC4)cccc3c2CC(F)(F)F)c(OCC(F)(F)F)c1 ; CN(C)CCNc1cccc2c(CC(F)(F)F)c(C#CCNc3ccc(S(N)(=O)=O)cc3OCC(F)(F)F)sc12]

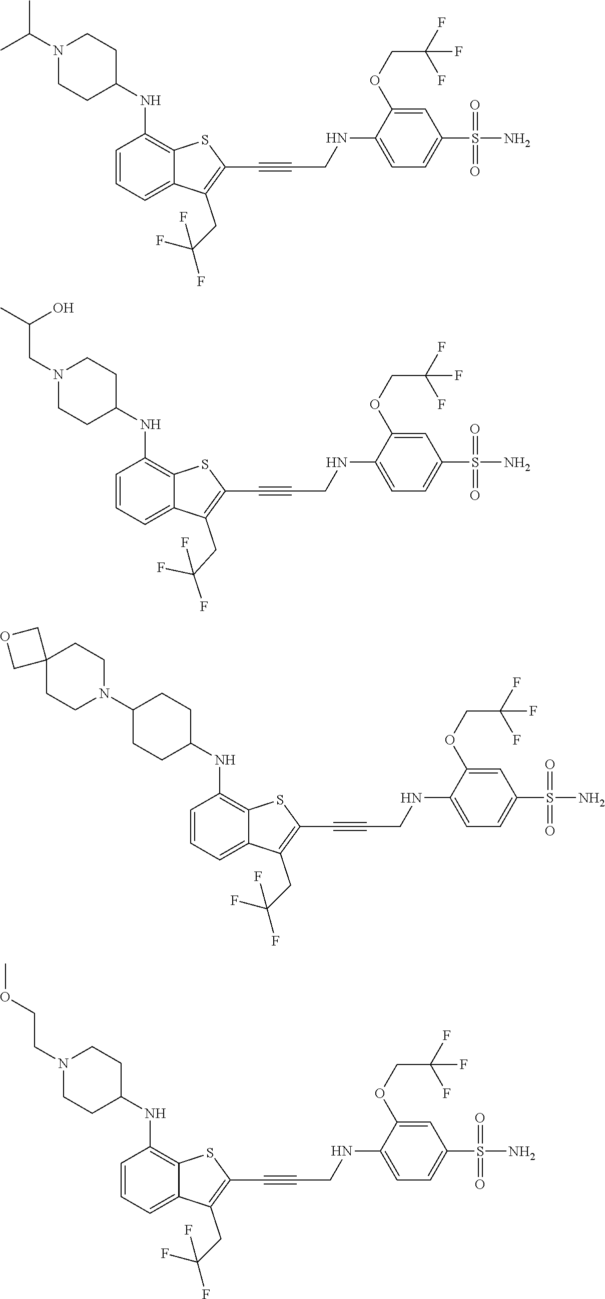 [SMILES: CC(C)N1CCC(Nc2cccc3c(CC(F)(F)F)c(C#CCNc4ccc(S(N)(=O)=O)cc4OCC(F)(F)F)sc23)CC1 ; CC(O)CN1CCC(Nc2cccc3c(CC(F)(F)F)c(C#CCNc4ccc(S(N)(=O)=O)cc4OCC(F)(F)F)sc23)CC1 ; NS(=O)(=O)c1ccc(NCC#Cc2sc3c(NC4CCC(N5CCC6(CC5)COC6)CC4)cccc3c2CC(F)(F)F)c(OCC(F)(F)F)c1 ; COCCN1CCC(Nc2cccc3c(CC(F)(F)F)c(C#CCNc4ccc(S(N)(=O)=O)cc4OCC(F)(F)F)sc23)CC1]

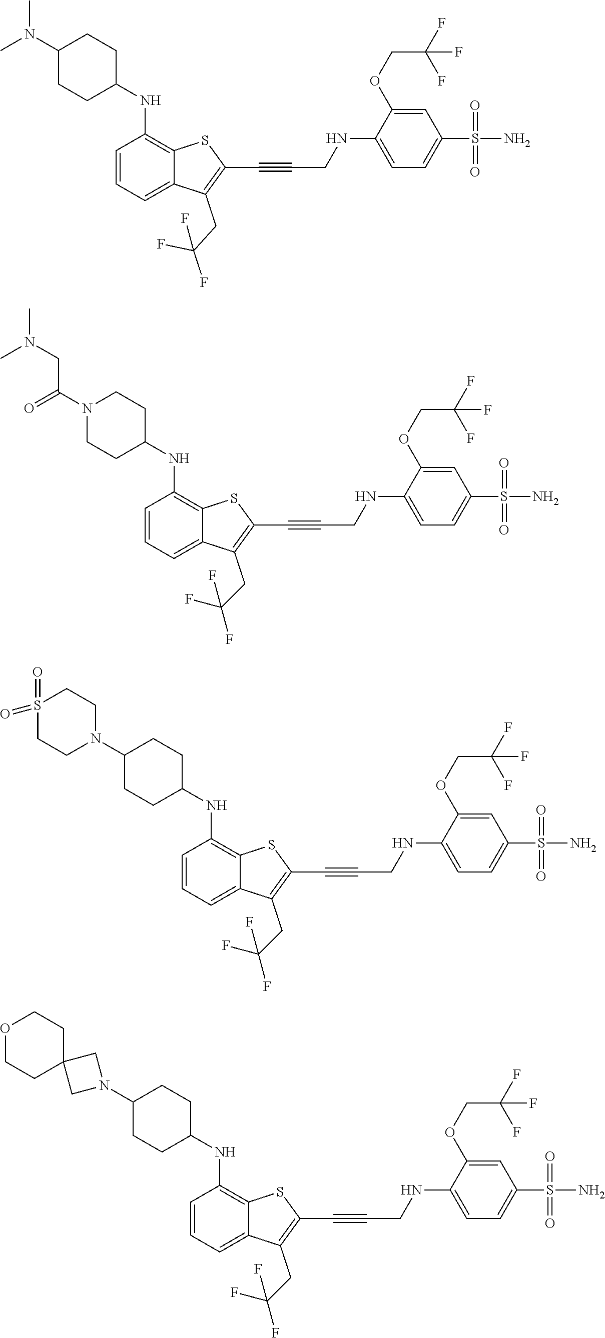 [SMILES: CN(C)C1CCC(Nc2cccc3c(CC(F)(F)F)c(C#CCNc4ccc(S(N)(=O)=O)cc4OCC(F)(F)F)sc23)CC1 ; CN(C)CC(=O)N1CCC(Nc2cccc3c(CC(F)(F)F)c(C#CCNc4ccc(S(N)(=O)=O)cc4OCC(F)(F)F)sc23)CC1 ; NS(=O)(=O)c1ccc(NCC#Cc2sc3c(NC4CCC(N5CCS(=O)(=O)CC5)CC4)cccc3c2CC(F)(F)F)c(OCC(F)(F)F)c1 ; NS(=O)(=O)c1ccc(NCC#Cc2sc3c(NC4CCC(N5CC6(CCOCC6)C5)CC4)cccc3c2CC(F)(F)F)c(OCC(F)(F)F)c1]

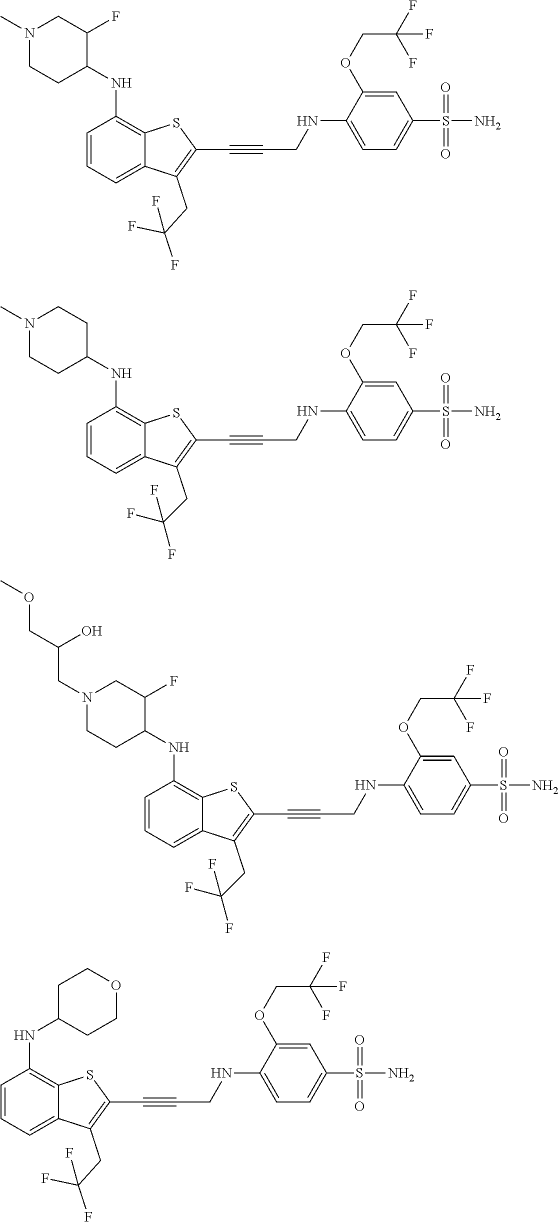 [SMILES: CN1CCC(Nc2cccc3c(CC(F)(F)F)c(C#CCNc4ccc(S(N)(=O)=O)cc4OCC(F)(F)F)sc23)C(F)C1 ; CN1CCC(Nc2cccc3c(CC(F)(F)F)c(C#CCNc4ccc(S(N)(=O)=O)cc4OCC(F)(F)F)sc23)CC1 ; COCC(O)CN1CCC(Nc2cccc3c(CC(F)(F)F)c(C#CCNc4ccc(S(N)(=O)=O)cc4OCC(F)(F)F)sc23)C(F)C1 ; NS(=O)(=O)c1ccc(NCC#Cc2sc3c(NC4CCOCC4)cccc3c2CC(F)(F)F)c(OCC(F)(F)F)c1]

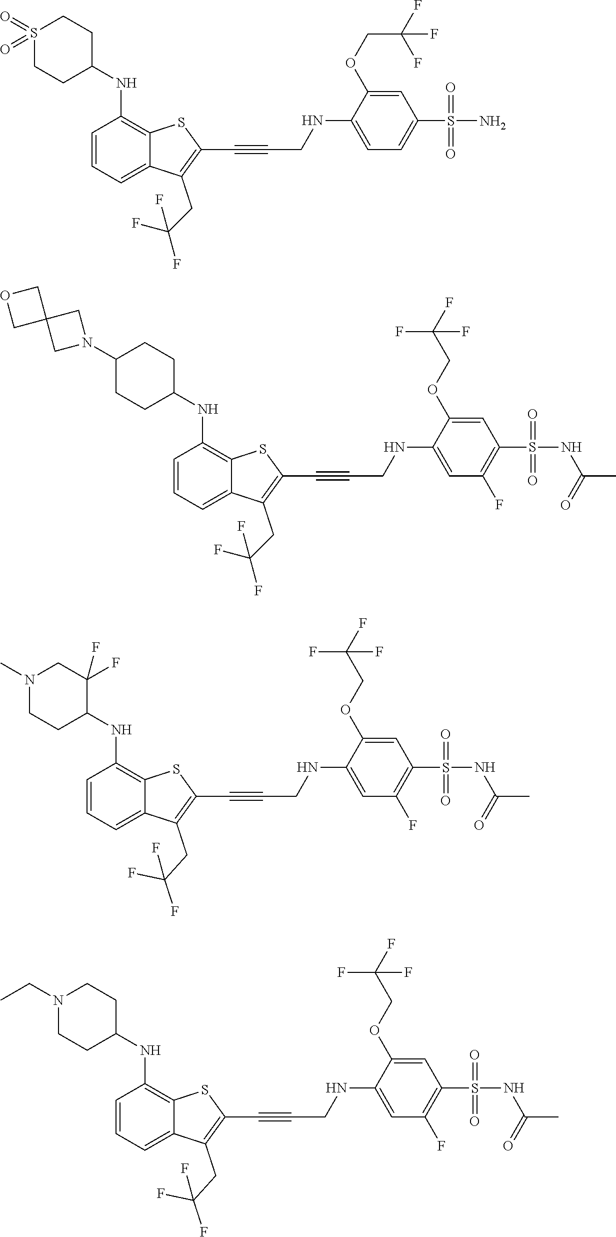

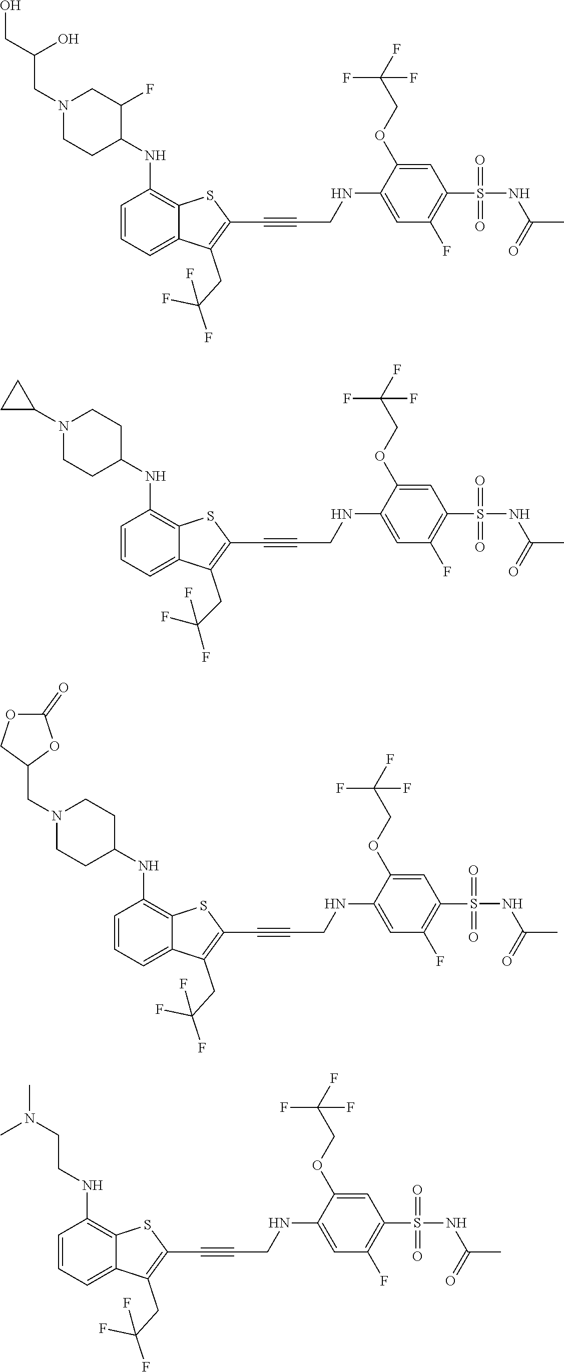

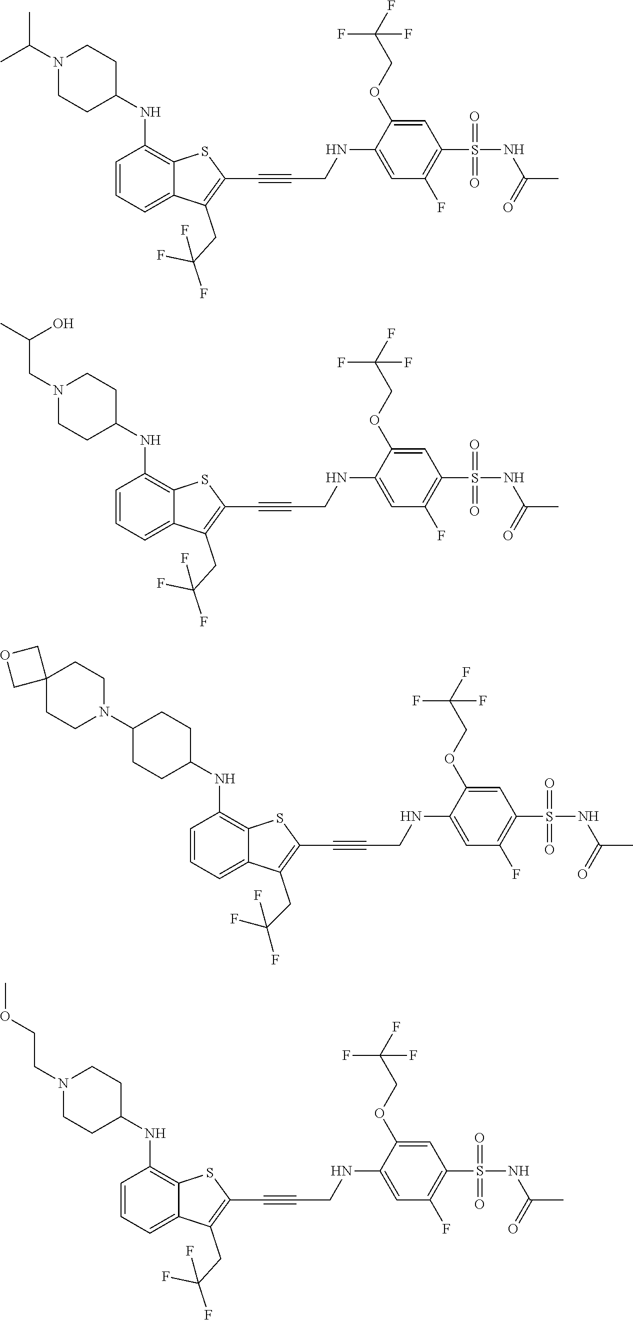

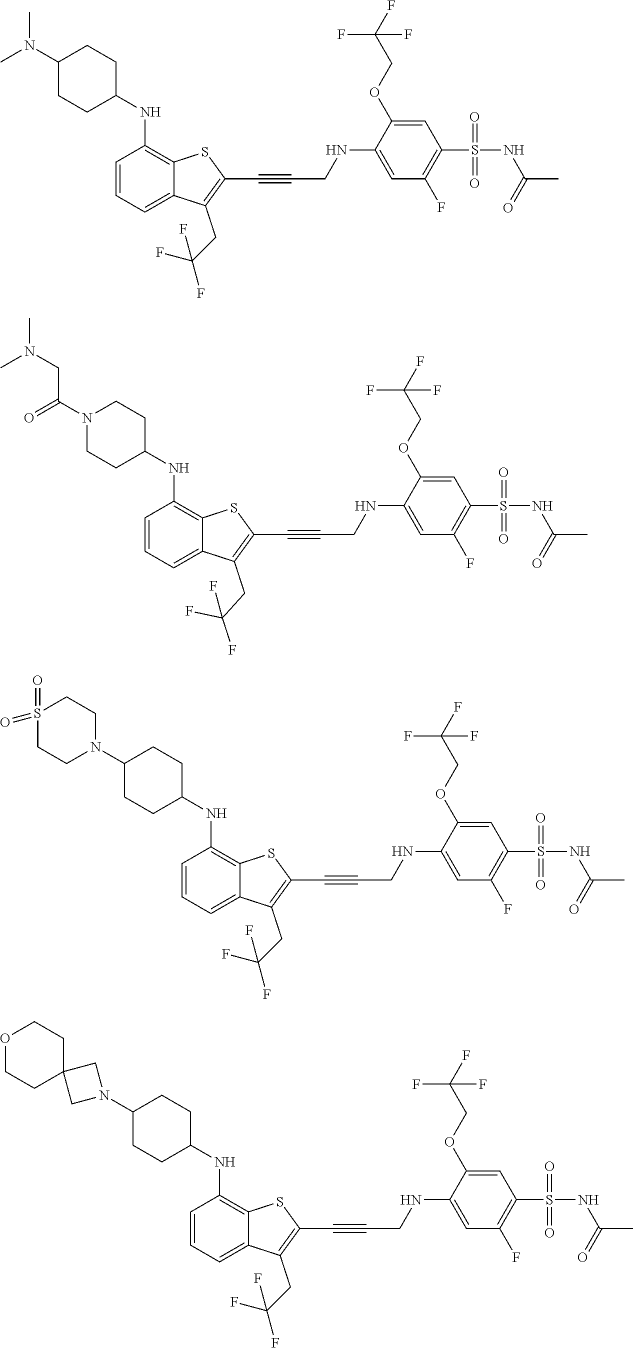

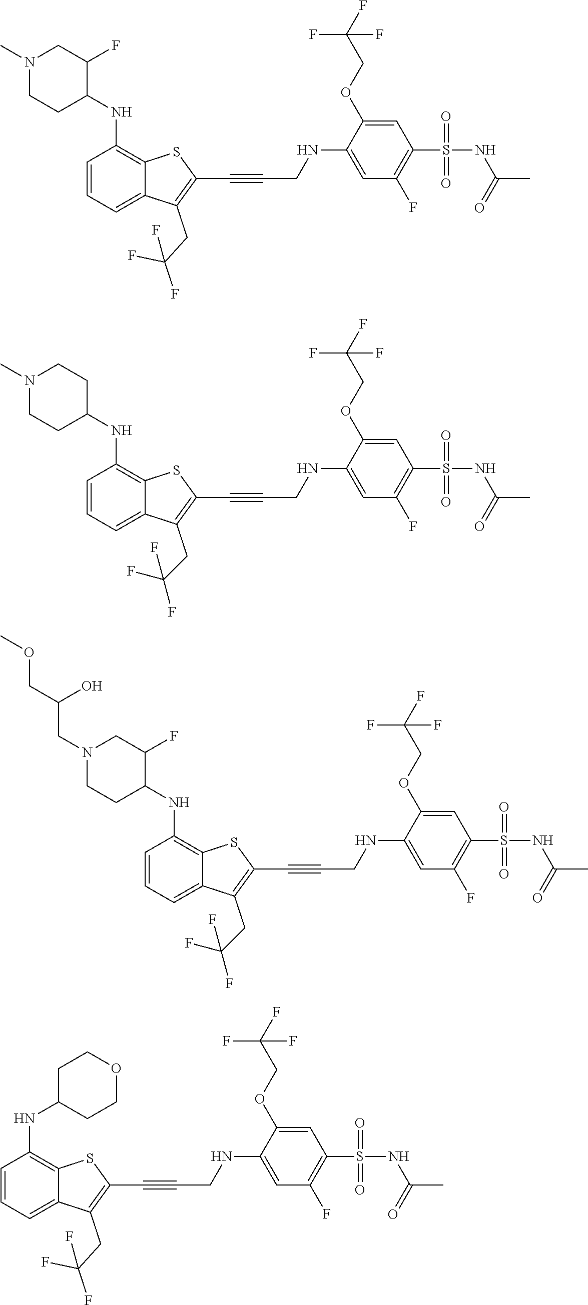

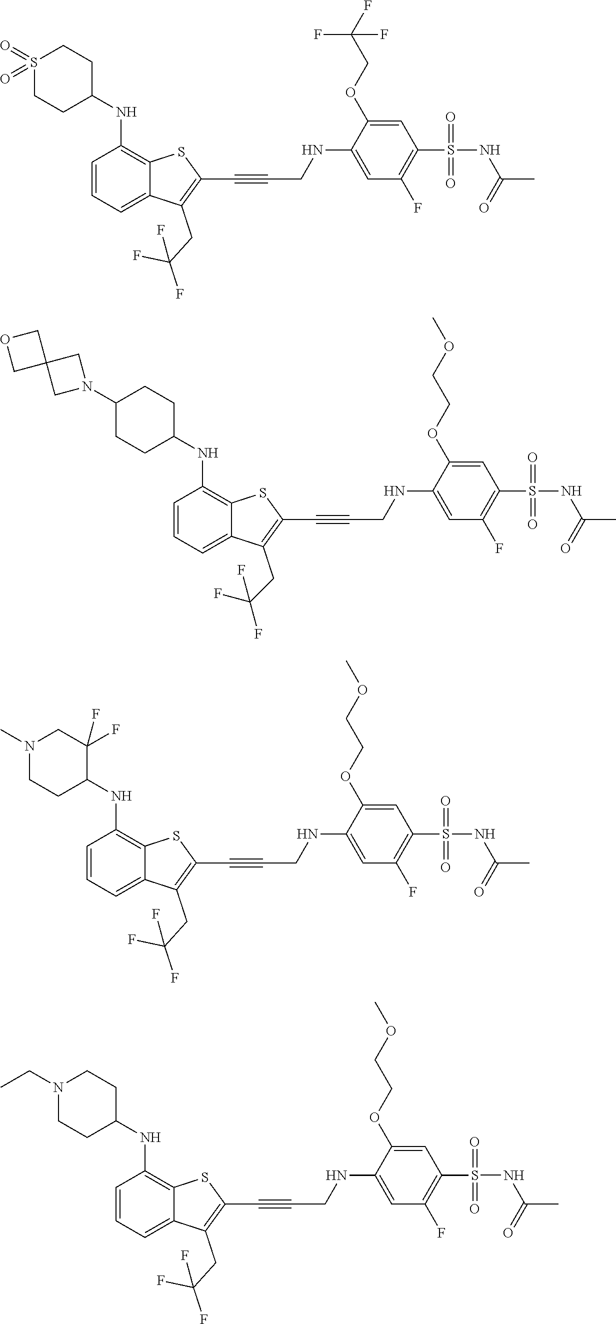

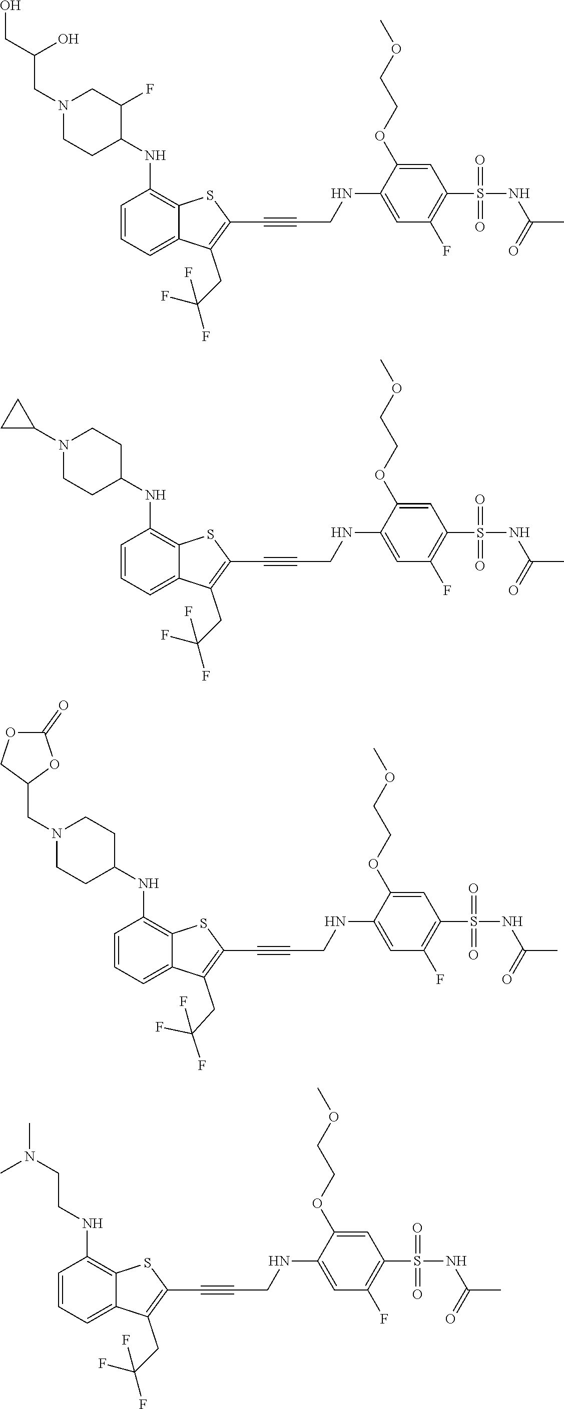

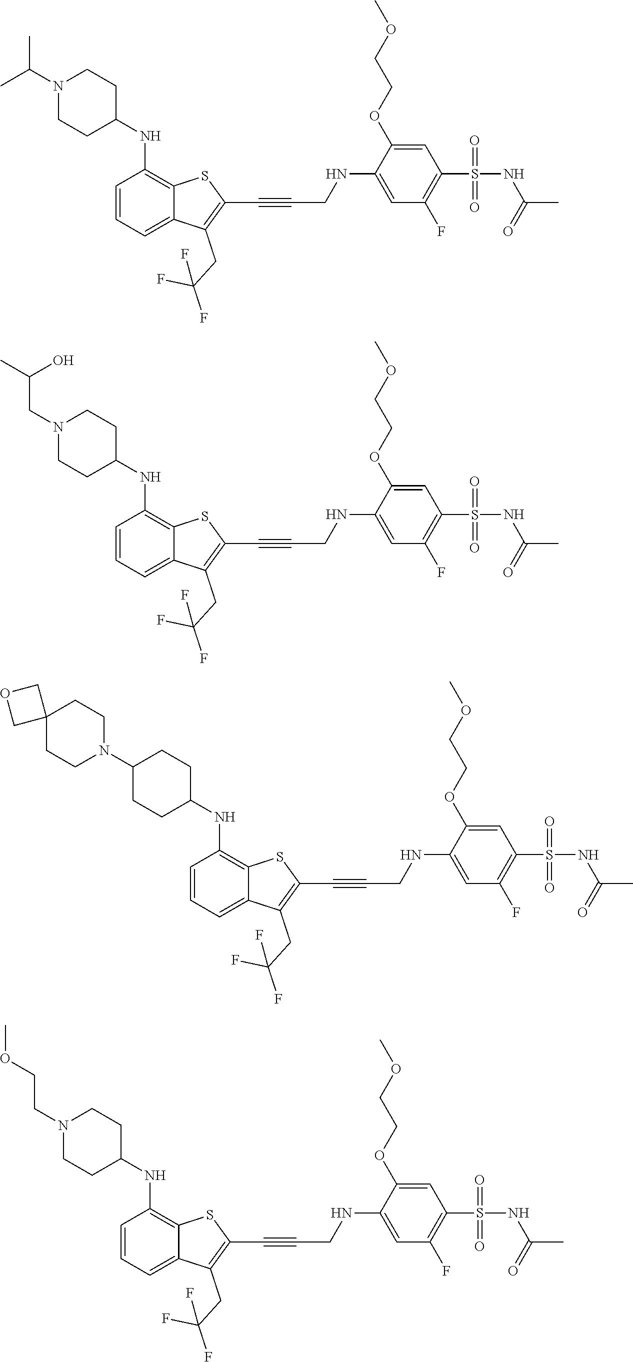

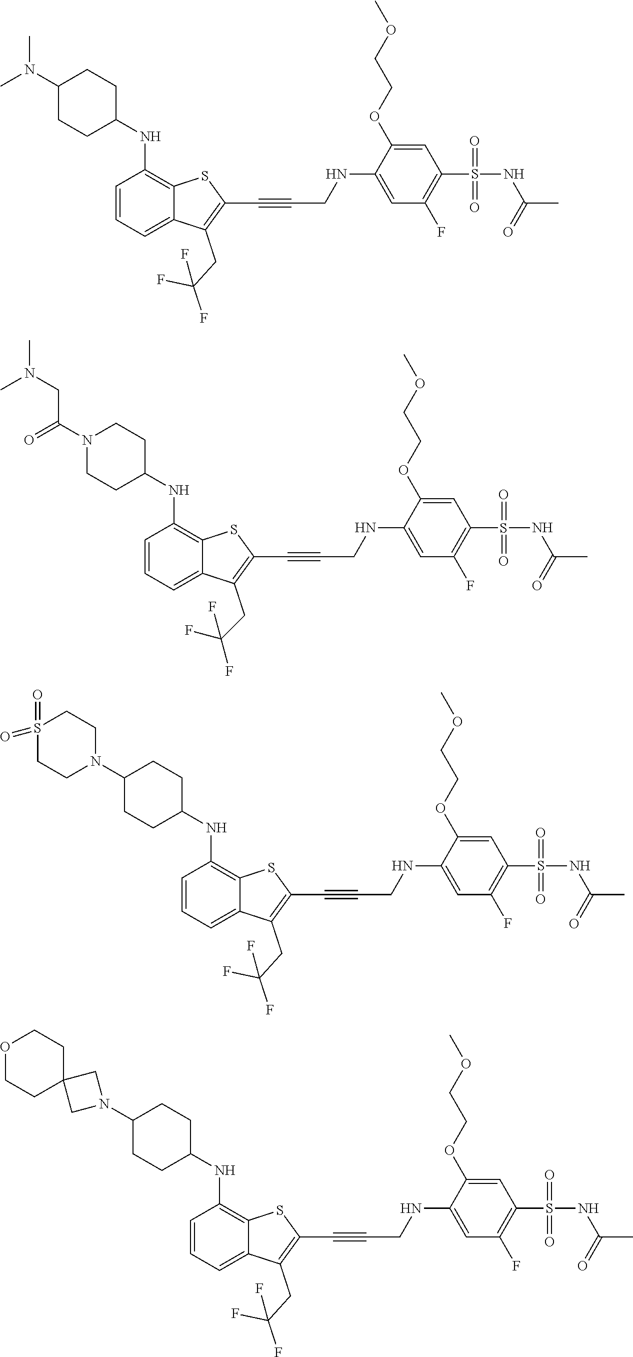

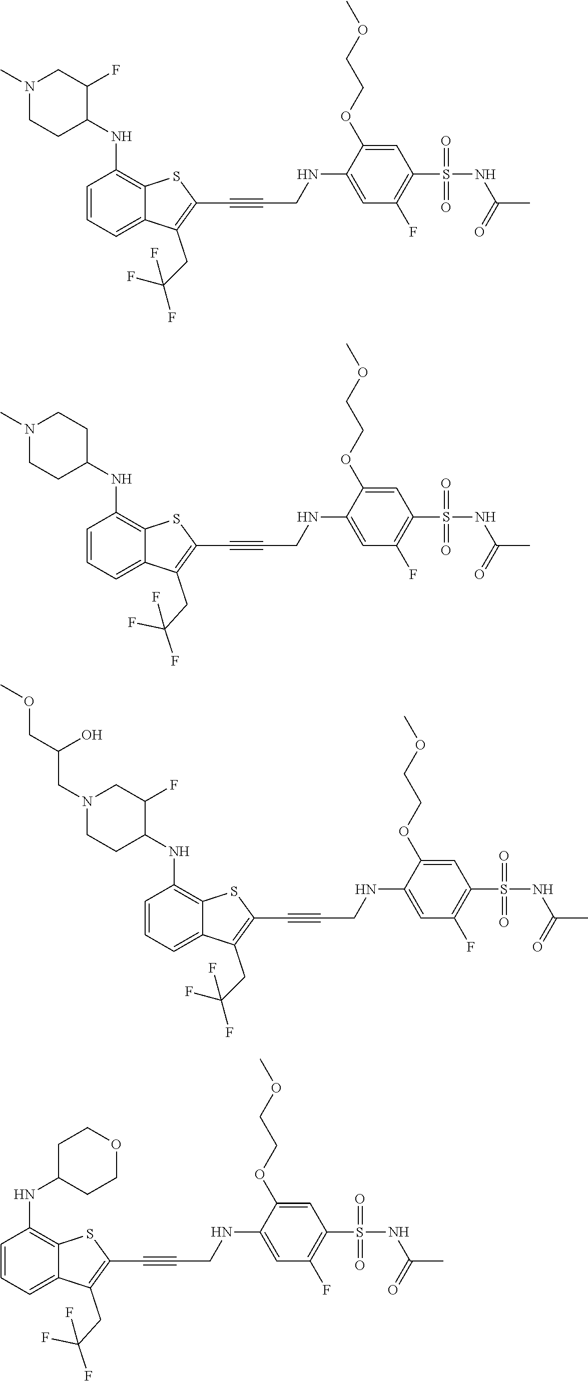

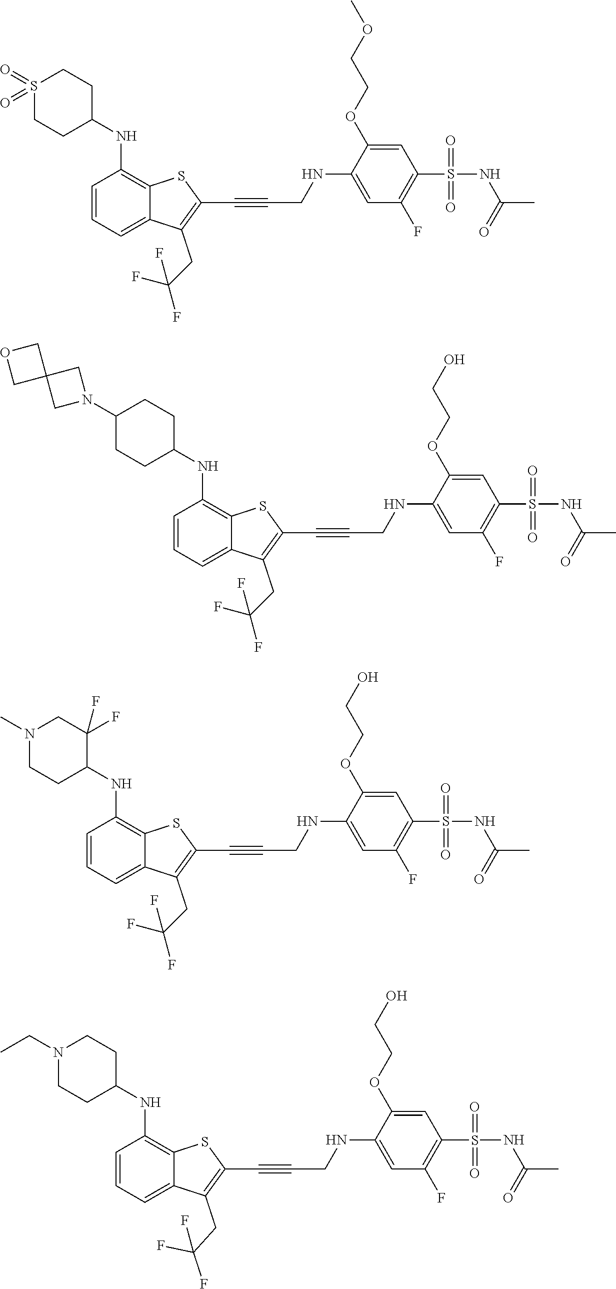

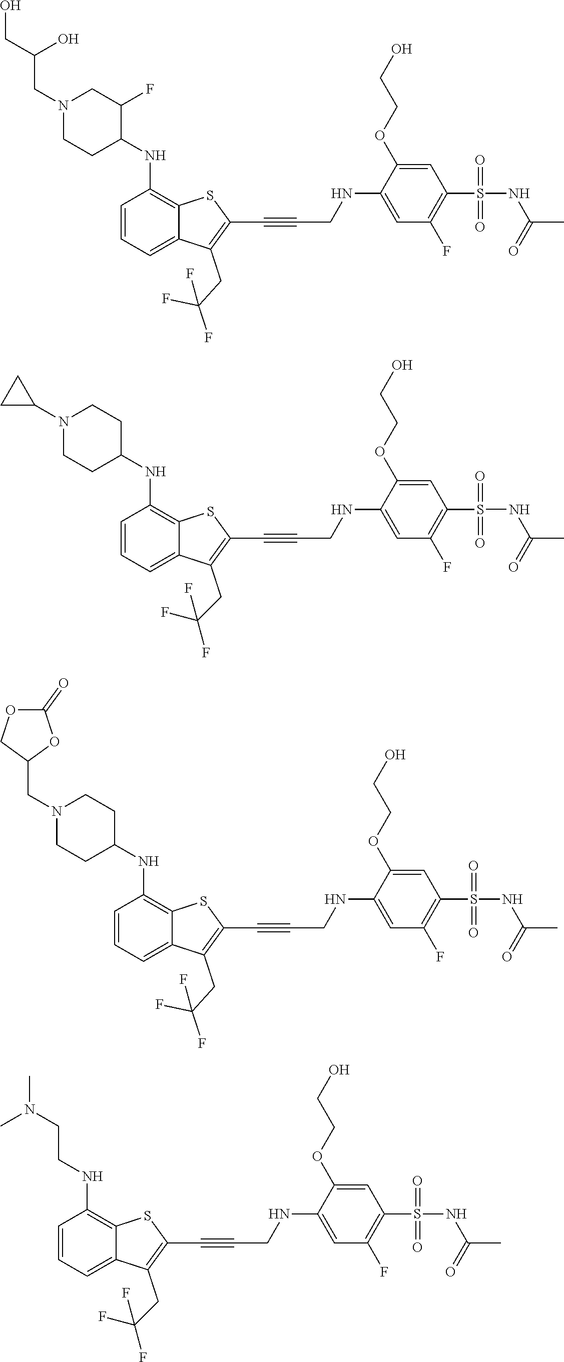

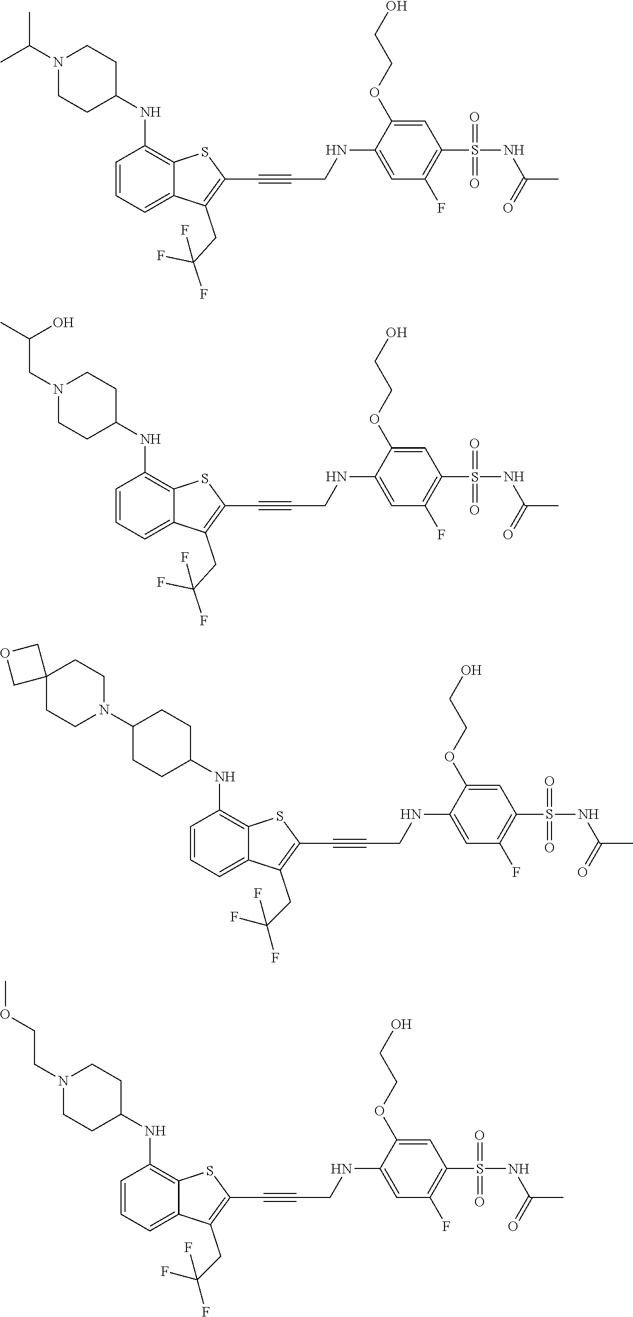

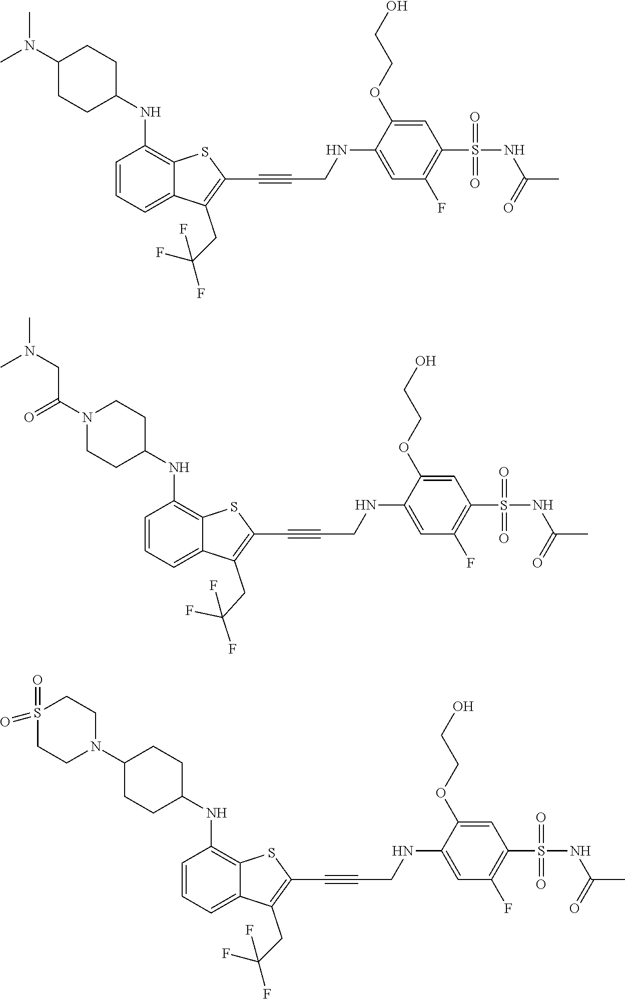

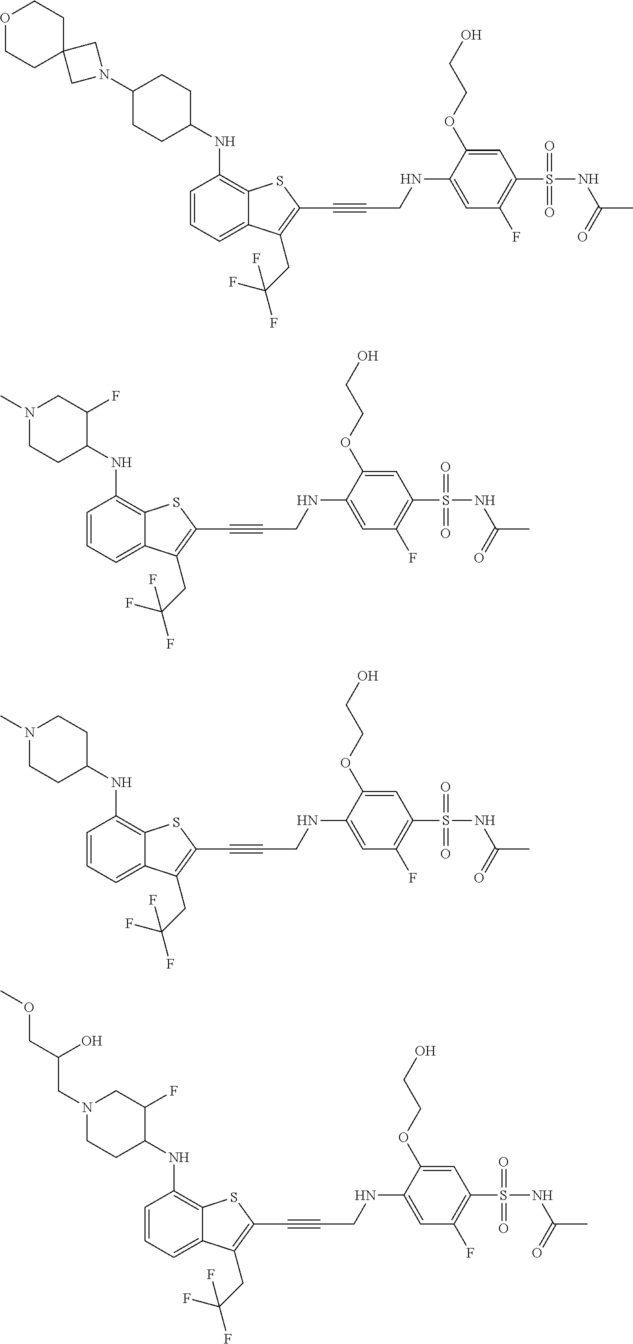

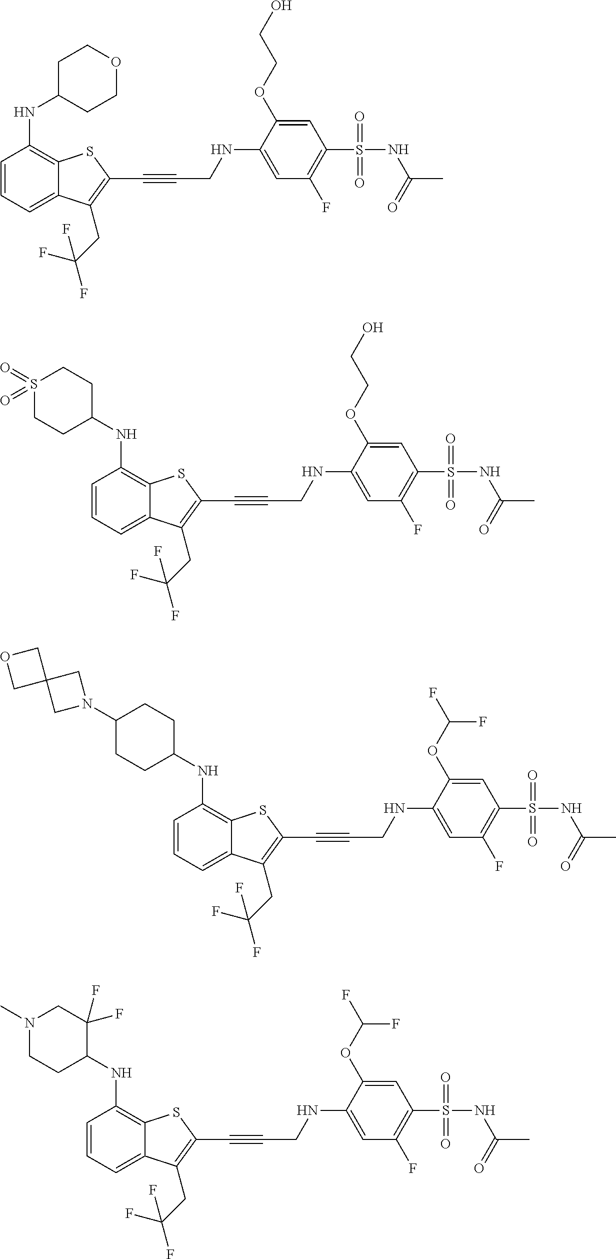

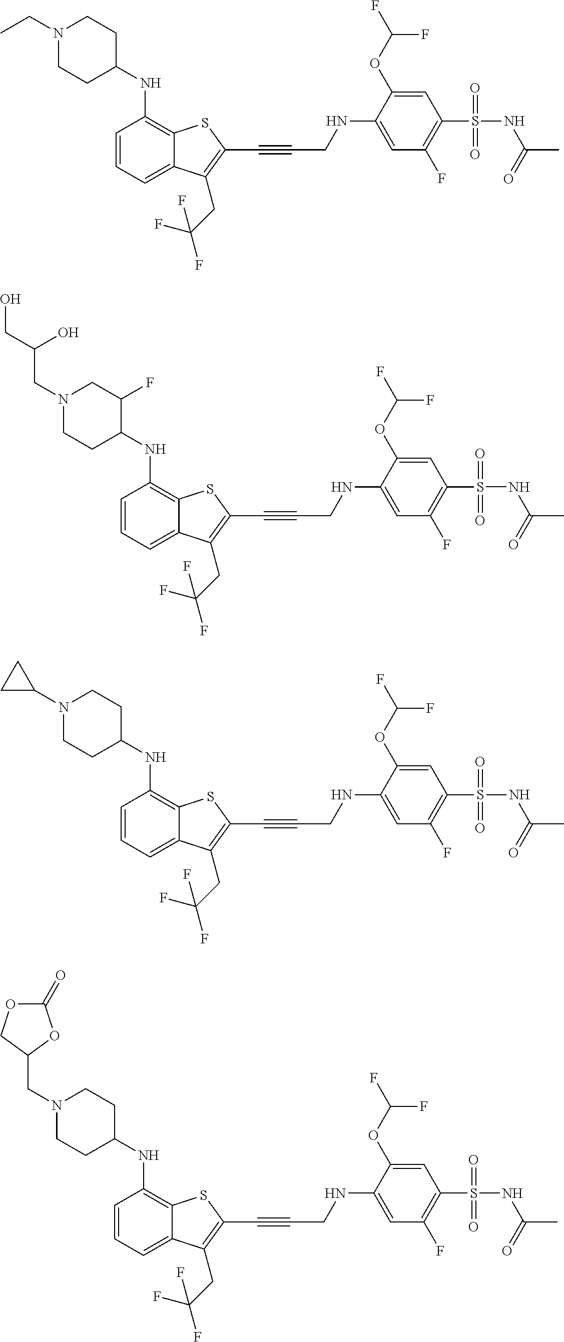 [SMILES: CCN1CCC(Nc2cccc3c(CC(F)(F)F)c(C#CCNc4cc(F)c(S(=O)(=O)NC(C)=O)cc4OC(F)F)sc23)CC1 ; CC(=O)NS(=O)(=O)c1cc(OC(F)F)c(NCC#Cc2sc3c(NC4CCN(CC(O)CO)CC4F)cccc3c2CC(F)(F)F)cc1F ; CC(=O)NS(=O)(=O)c1cc(OC(F)F)c(NCC#Cc2sc3c(NC4CCN(C5CC5)CC4)cccc3c2CC(F)(F)F)cc1F ; CC(=O)NS(=O)(=O)c1cc(OC(F)F)c(NCC#Cc2sc3c(NC4CCN(CC5COC(=O)O5)CC4)cccc3c2CC(F)(F)F)cc1F]

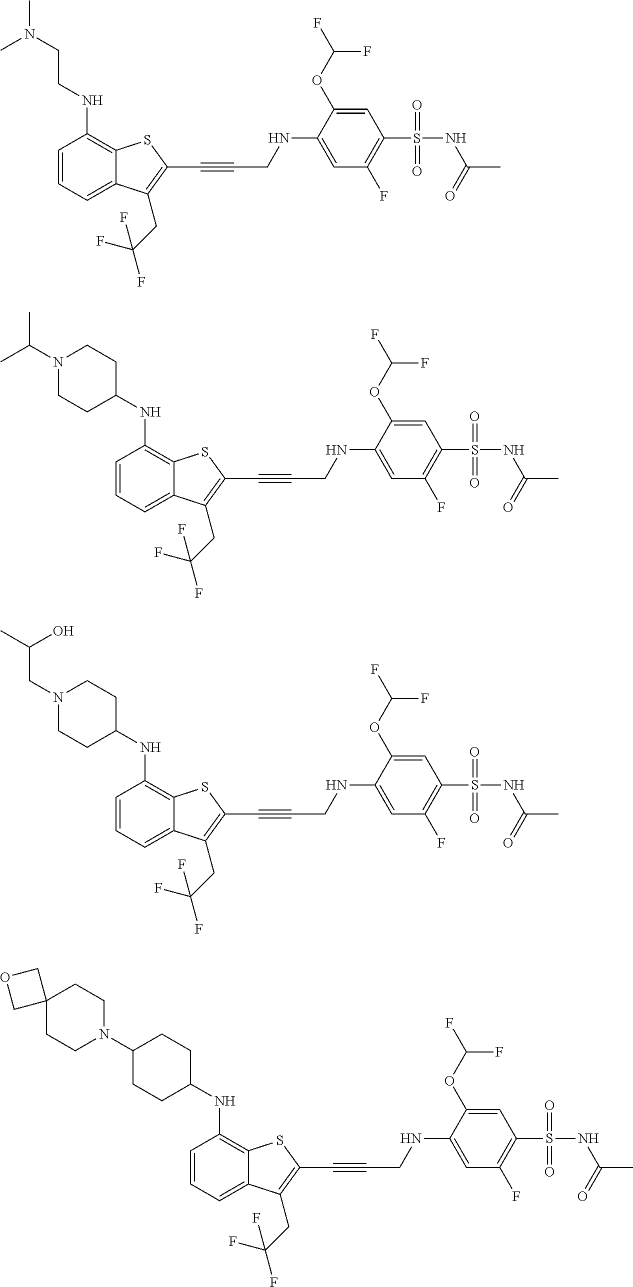

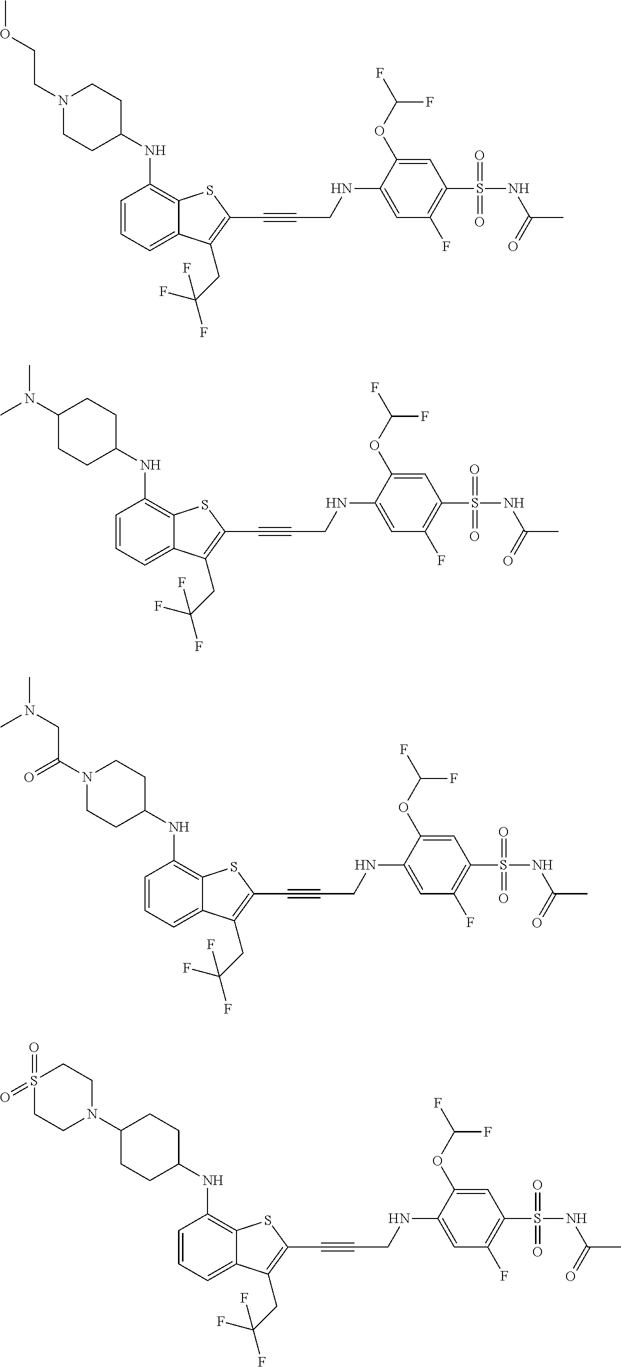

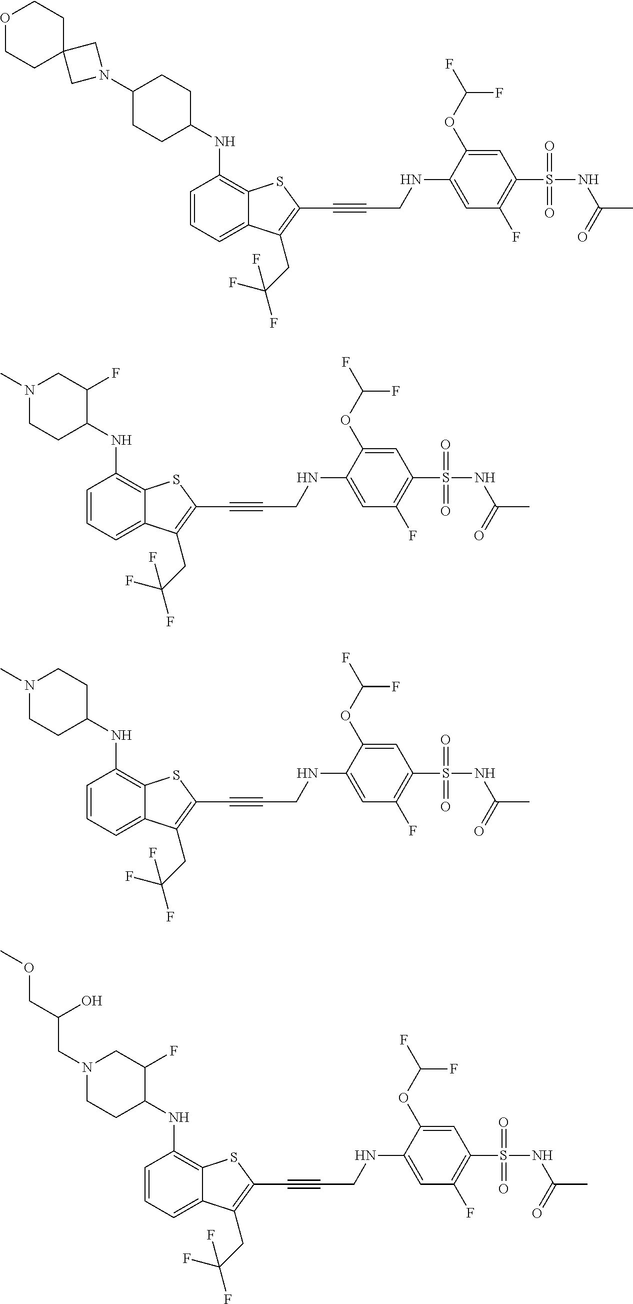

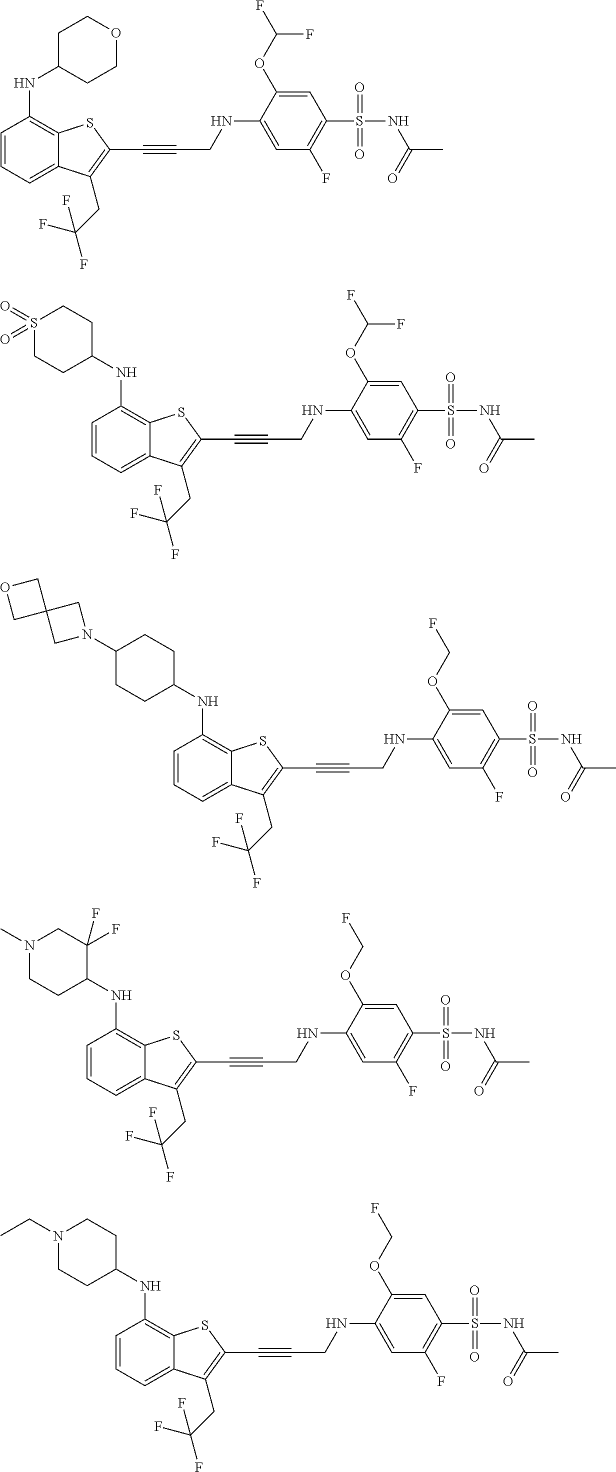

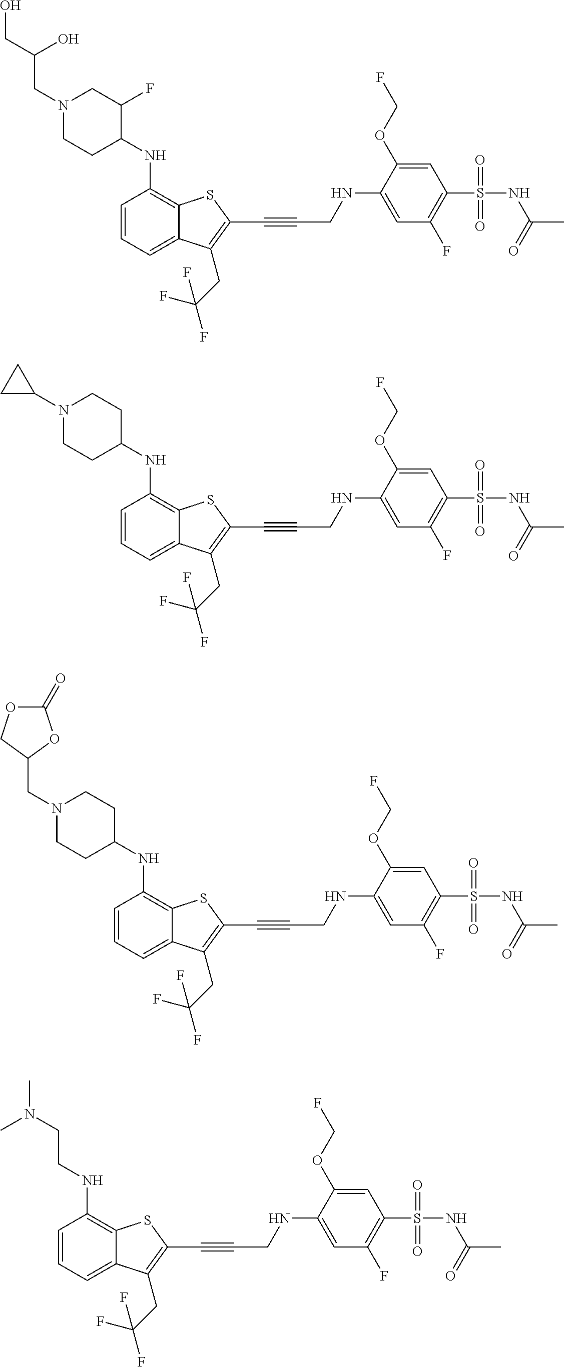

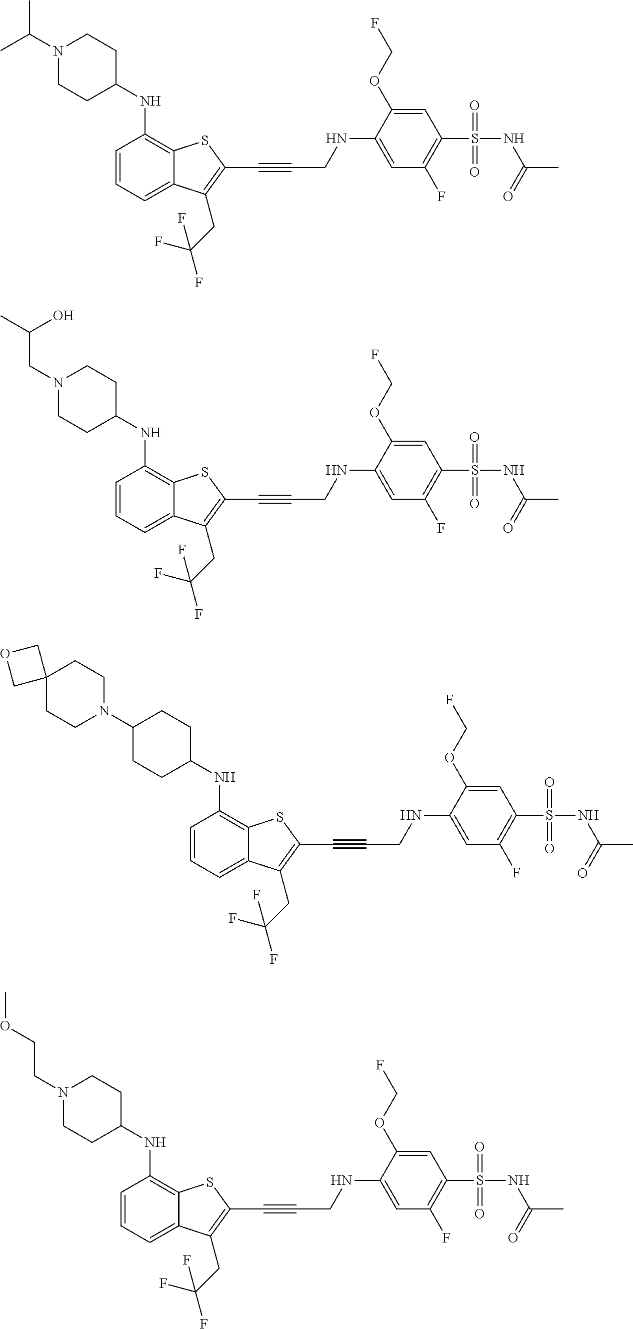

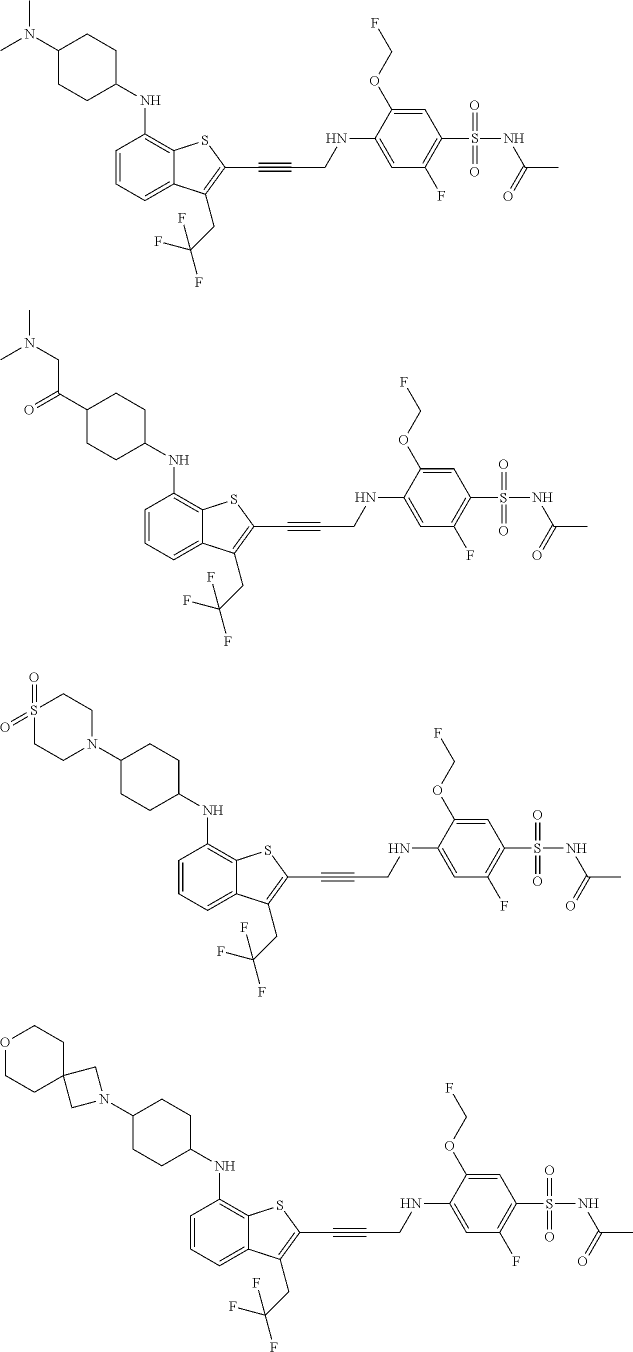

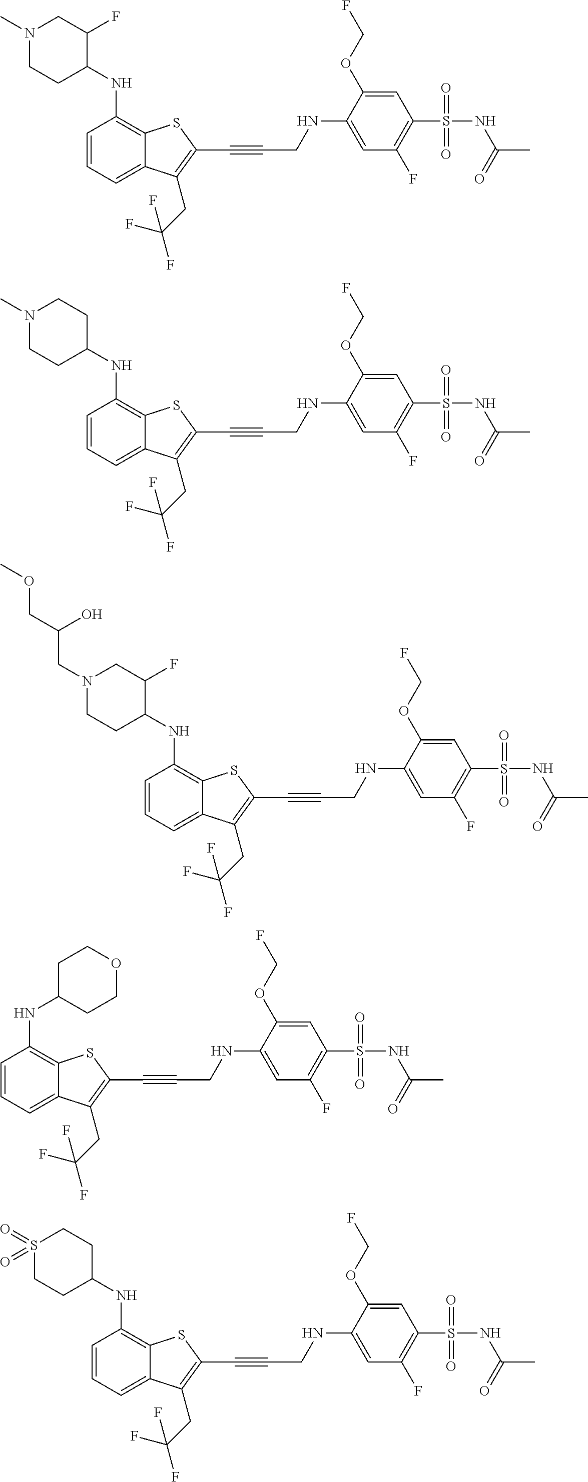

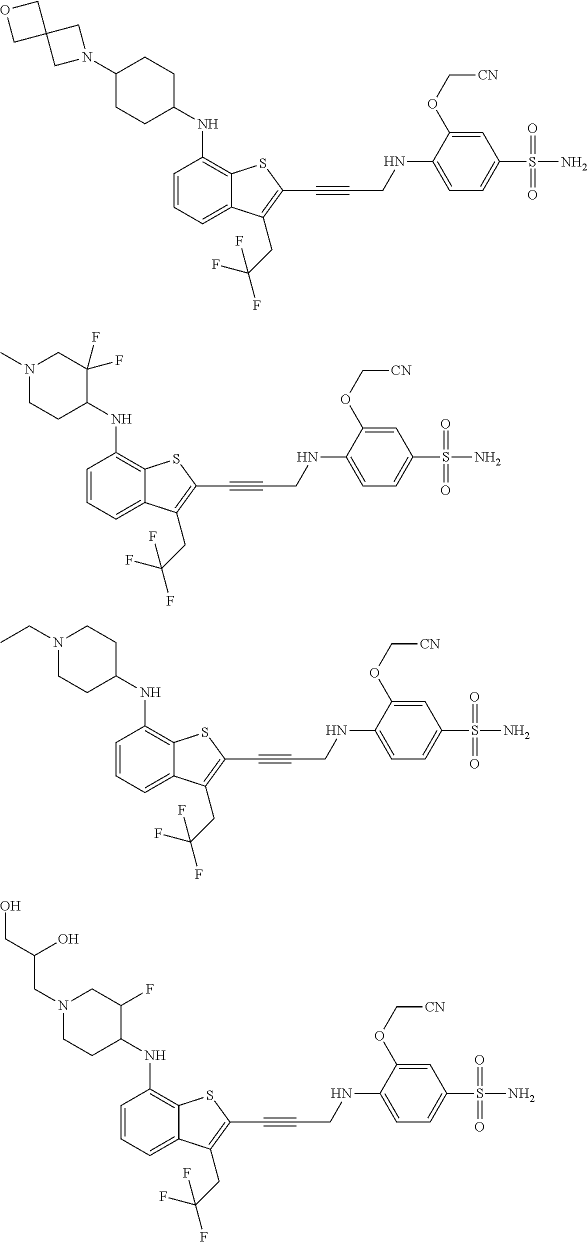 [SMILES: N#CCOc1cc(S(N)(=O)=O)ccc1NCC#Cc1sc2c(NC3CCC(N4CC5(COC5)C4)CC3)cccc2c1CC(F)(F)F ; CN1CCC(Nc2cccc3c(CC(F)(F)F)c(C#CCNc4ccc(S(N)(=O)=O)cc4OCC#N)sc23)C(F)(F)C1 ; CCN1CCC(Nc2cccc3c(CC(F)(F)F)c(C#CCNc4ccc(S(N)(=O)=O)cc4OCC#N)sc23)CC1 ; N#CCOc1cc(S(N)(=O)=O)ccc1NCC#Cc1sc2c(NC3CCN(CC(O)CO)CC3F)cccc2c1CC(F)(F)F]

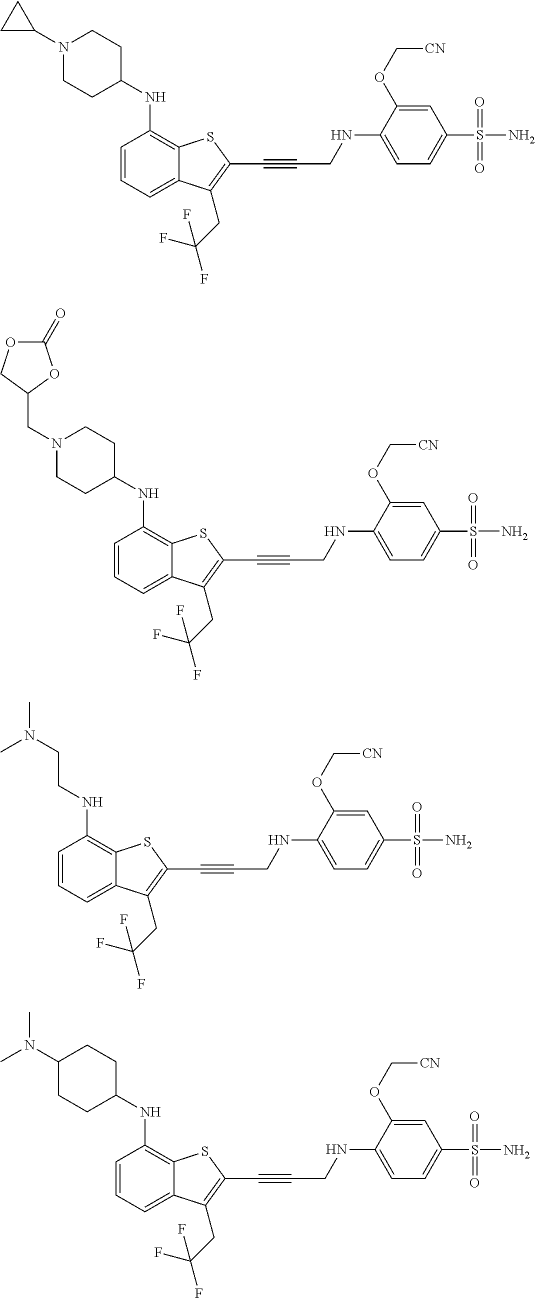 [SMILES: N#CCOc1cc(S(N)(=O)=O)ccc1NCC#Cc1sc2c(NC3CCN(C4CC4)CC3)cccc2c1CC(F)(F)F ; N#CCOc1cc(S(N)(=O)=O)ccc1NCC#Cc1sc2c(NC3CCN(CC4COC(=O)O4)CC3)cccc2c1CC(F)(F)F ; CN(C)CCNc1cccc2c(CC(F)(F)F)c(C#CCNc3ccc(S(N)(=O)=O)cc3OCC#N)sc12 ; CN(C)C1CCC(Nc2cccc3c(CC(F)(F)F)c(C#CCNc4ccc(S(N)(=O)=O)cc4OCC#N)sc23)CC1]

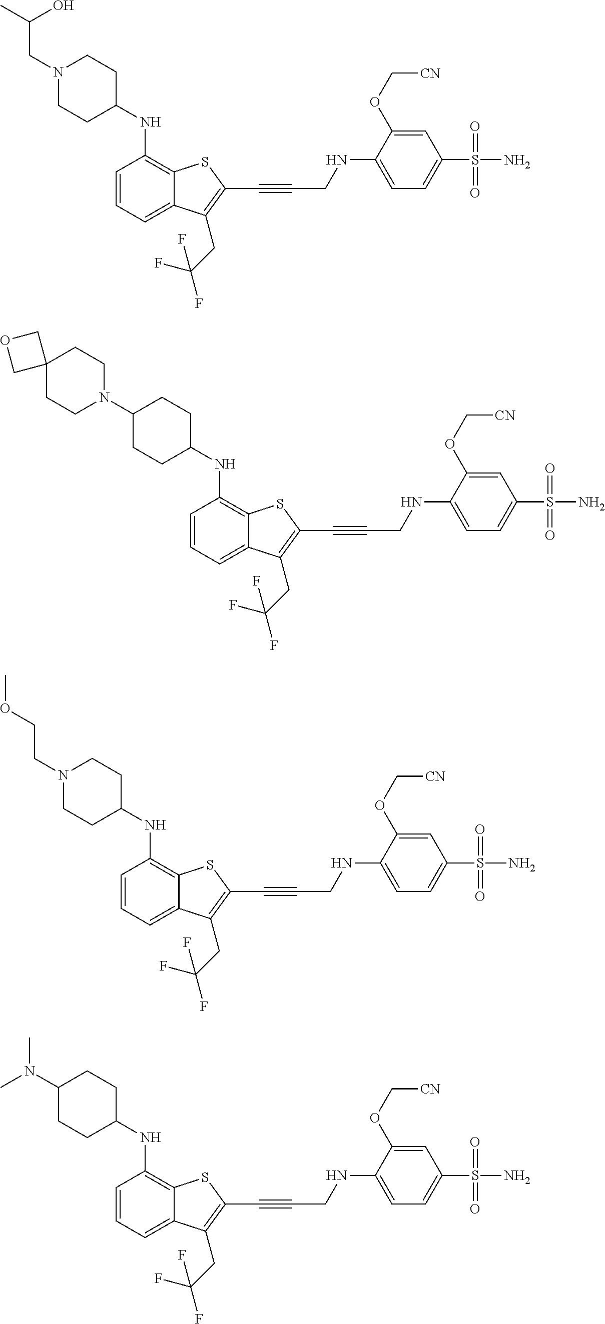 [SMILES: CC(O)CN1CCC(Nc2cccc3c(CC(F)(F)F)c(C#CCNc4ccc(S(N)(=O)=O)cc4OCC#N)sc23)CC1 ; N#CCOc1cc(S(N)(=O)=O)ccc1NCC#Cc1sc2c(NC3CCC(N4CCC5(CC4)COC5)CC3)cccc2c1CC(F)(F)F ; COCCN1CCC(Nc2cccc3c(CC(F)(F)F)c(C#CCNc4ccc(S(N)(=O)=O)cc4OCC#N)sc23)CC1 ; CN(C)C1CCC(Nc2cccc3c(CC(F)(F)F)c(C#CCNc4ccc(S(N)(=O)=O)cc4OCC#N)sc23)CC1]

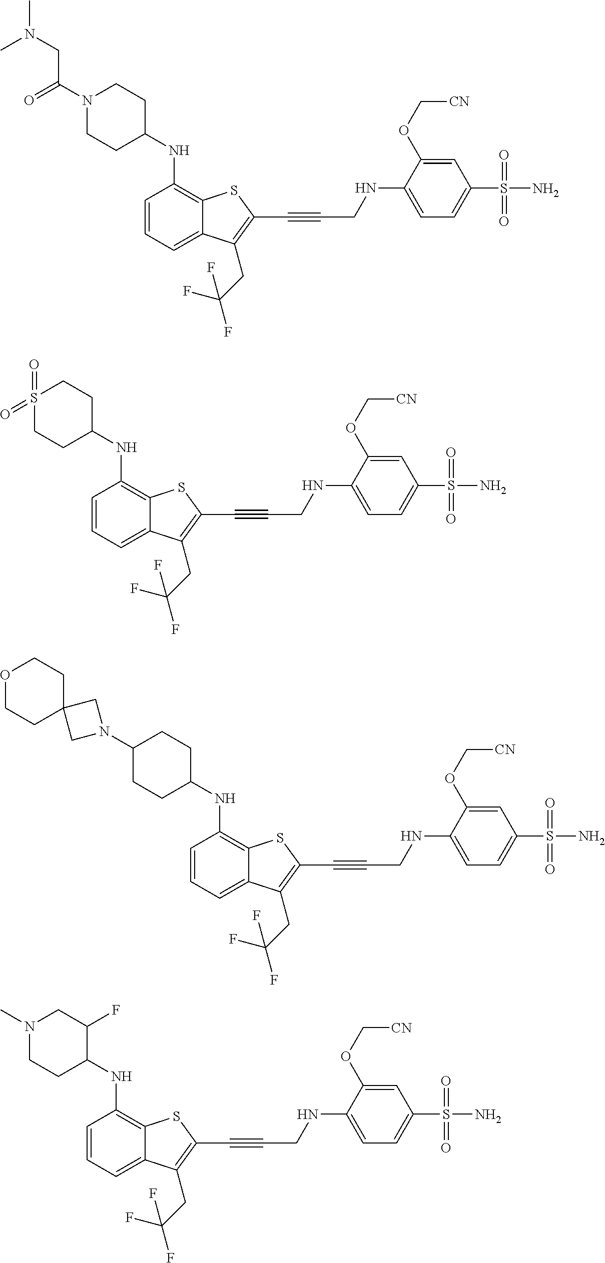 [SMILES: CN(C)CC(=O)N1CCC(Nc2cccc3c(CC(F)(F)F)c(C#CCNc4ccc(S(N)(=O)=O)cc4OCC#N)sc23)CC1 ; N#CCOc1cc(S(N)(=O)=O)ccc1NCC#Cc1sc2c(NC3CCS(=O)(=O)CC3)cccc2c1CC(F)(F)F ; N#CCOc1cc(S(N)(=O)=O)ccc1NCC#Cc1sc2c(NC3CCC(N4CC5(CCOCC5)C4)CC3)cccc2c1CC(F)(F)F ; CN1CCC(Nc2cccc3c(CC(F)(F)F)c(C#CCNc4ccc(S(N)(=O)=O)cc4OCC#N)sc23)C(F)C1]

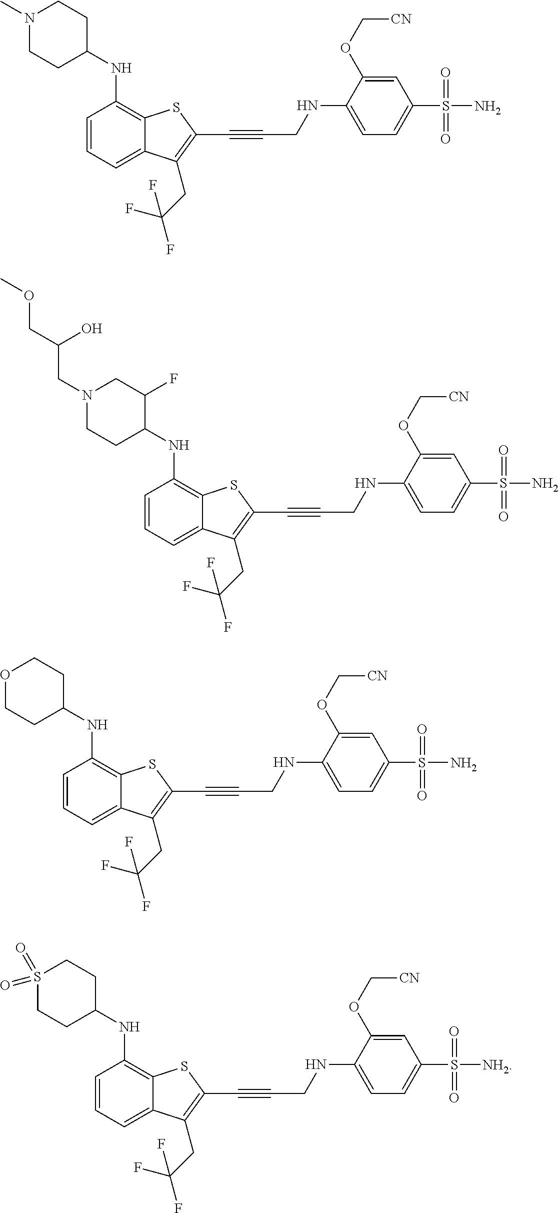 [SMILES: CN1CCC(Nc2cccc3c(CC(F)(F)F)c(C#CCNc4ccc(S(N)(=O)=O)cc4OCC#N)sc23)CC1 ; COCC(O)CN1CCC(Nc2cccc3c(CC(F)(F)F)c(C#CCNc4ccc(S(N)(=O)=O)cc4OCC#N)sc23)C(F)C1 ; N#CCOc1cc(S(N)(=O)=O)ccc1NCC#Cc1sc2c(NC3CCOCC3)cccc2c1CC(F)(F)F ; N#CCOc1cc(S(N)(=O)=O)ccc1NCC#Cc1sc2c(NC3CCS(=O)(=O)CC3)cccc2c1CC(F)(F)F]

[56]. The compound according to any one of [1] to [53], wherein the compound of formula (I) is selected from:

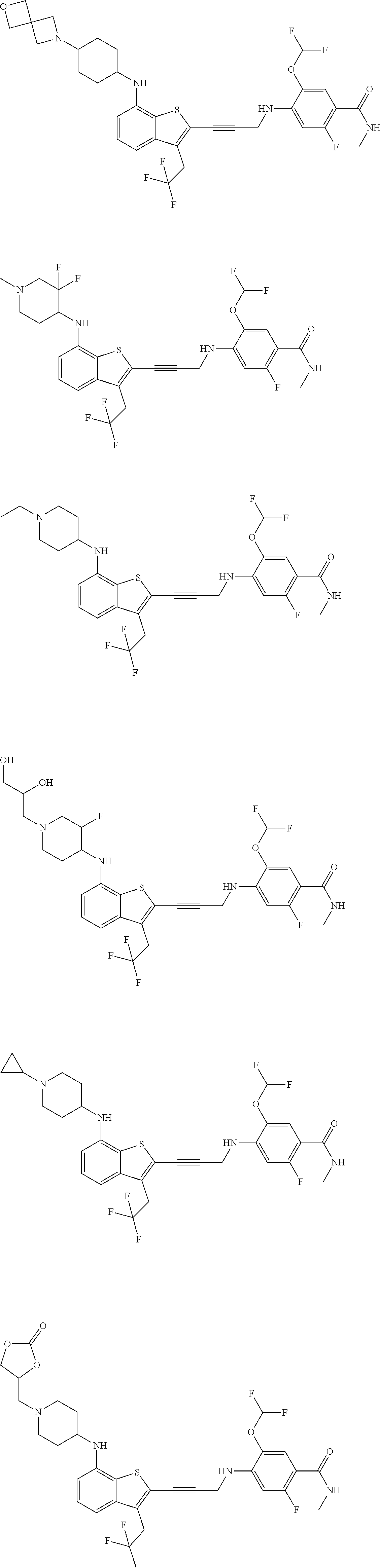 [SMILES: CNC(=O)c1cc(OC(F)F)c(NCC#Cc2sc3c(NC4CCC(N5CC6(COC6)C5)CC4)cccc3c2CC(F)(F)F)cc1F ; CNC(=O)c1cc(OC(F)F)c(NCC#Cc2sc3c(NC4CCN(C)CC4(F)F)cccc3c2CC(F)(F)F)cc1F ; CCN1CCC(Nc2cccc3c(CC(F)(F)F)c(C#CCNc4cc(F)c(C(=O)NC)cc4OC(F)F)sc23)CC1 ; CNC(=O)c1cc(OC(F)F)c(NCC#Cc2sc3c(NC4CCN(CC(O)CO)CC4F)cccc3c2CC(F)(F)F)cc1F ; CNC(=O)c1cc(OC(F)F)c(NCC#Cc2sc3c(NC4CCN(C5CC5)CC4)cccc3c2CC(F)(F)F)cc1F ; CNC(=O)c1cc(OC(F)F)c(NCC#Cc2sc3c(NC4CCN(CC5COC(=O)O5)CC4)cccc3c2CC(C)(F)F)cc1F]

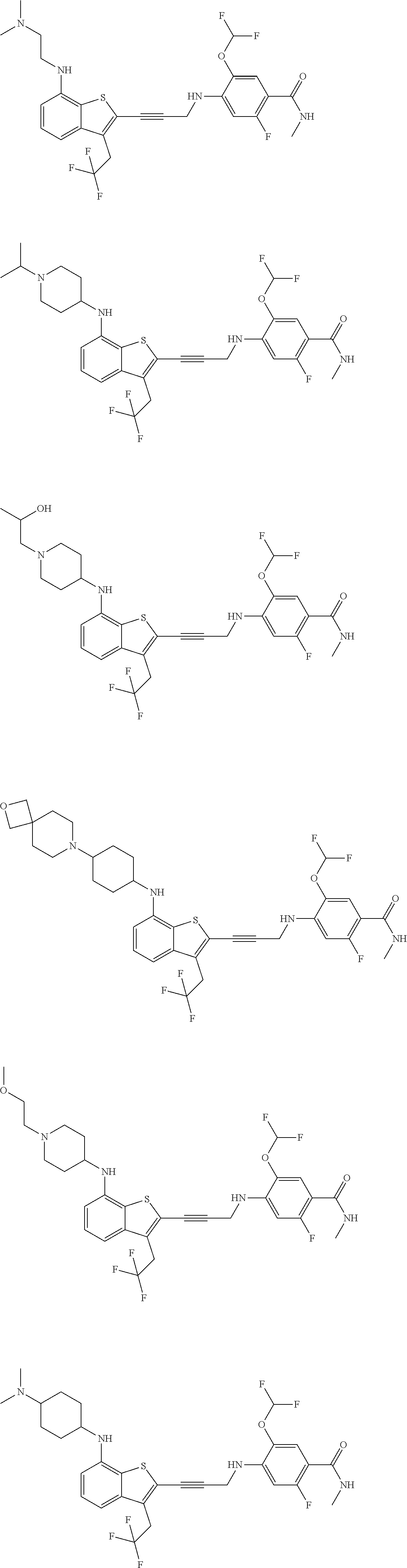 [SMILES: CNC(=O)c1cc(OC(F)F)c(NCC#Cc2sc3c(NCCN(C)C)cccc3c2CC(F)(F)F)cc1F ; CNC(=O)c1cc(OC(F)F)c(NCC#Cc2sc3c(NC4CCN(C(C)C)CC4)cccc3c2CC(F)(F)F)cc1F ; CNC(=O)c1cc(OC(F)F)c(NCC#Cc2sc3c(NC4CCN(CC(C)O)CC4)cccc3c2CC(F)(F)F)cc1F ; CNC(=O)c1cc(OC(F)F)c(NCC#Cc2sc3c(NC4CCC(N5CCC6(CC5)COC6)CC4)cccc3c2CC(F)(F)F)cc1F ; CNC(=O)c1cc(OC(F)F)c(NCC#Cc2sc3c(NC4CCN(CCOC)CC4)cccc3c2CC(F)(F)F)cc1F ; CNC(=O)c1cc(OC(F)F)c(NCC#Cc2sc3c(NC4CCC(N(C)C)CC4)cccc3c2CC(F)(F)F)cc1F]

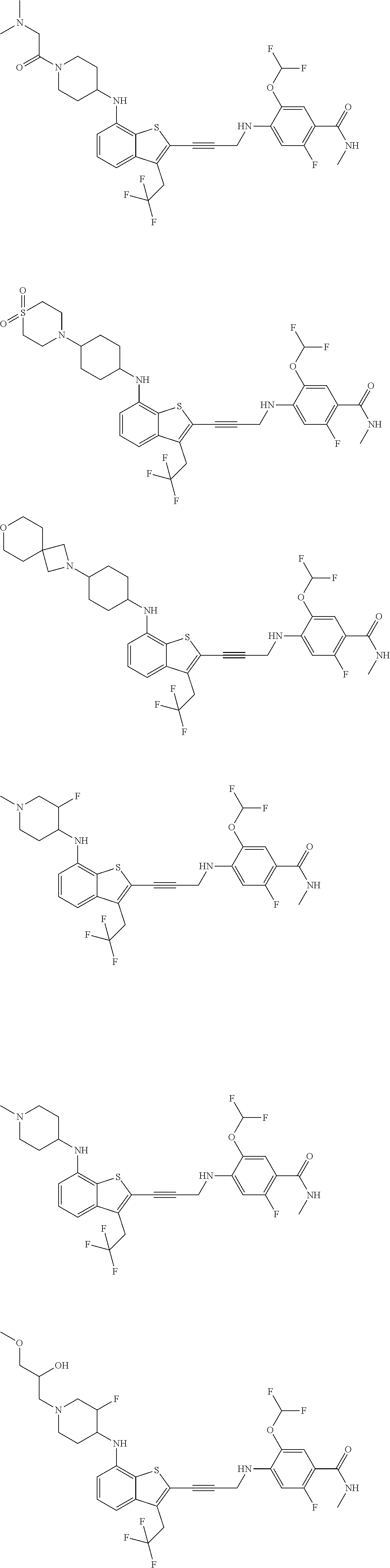 [SMILES: CNC(=O)c1cc(OC(F)F)c(NCC#Cc2sc3c(NC4CCN(C(=O)CN(C)C)CC4)cccc3c2CC(F)(F)F)cc1F ; CNC(=O)c1cc(OC(F)F)c(NCC#Cc2sc3c(NC4CCC(N5CCS(=O)(=O)CC5)CC4)cccc3c2CC(F)(F)F)cc1F ; CNC(=O)c1cc(OC(F)F)c(NCC#Cc2sc3c(NC4CCC(N5CC6(CCOCC6)C5)CC4)cccc3c2CC(F)(F)F)cc1F ; CNC(=O)c1cc(OC(F)F)c(NCC#Cc2sc3c(NC4CCN(C)CC4F)cccc3c2CC(F)(F)F)cc1F ; CNC(=O)c1cc(OC(F)F)c(NCC#Cc2sc3c(NC4CCN(C)CC4)cccc3c2CC(F)(F)F)cc1F ; CNC(=O)c1cc(OC(F)F)c(NCC#Cc2sc3c(NC4CCN(CC(O)COC)CC4F)cccc3c2CC(F)(F)F)cc1F]

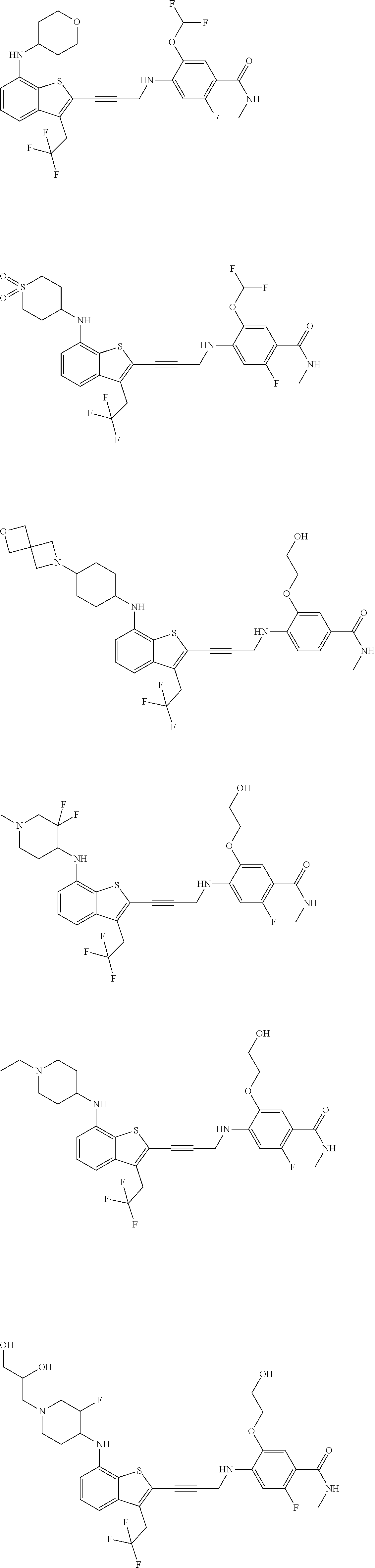 [SMILES: CNC(=O)c1cc(OC(F)F)c(NCC#Cc2sc3c(NC4CCOCC4)cccc3c2CC(F)(F)F)cc1F ; CNC(=O)c1cc(OC(F)F)c(NCC#Cc2sc3c(NC4CCS(=O)(=O)CC4)cccc3c2CC(F)(F)F)cc1F ; CNC(=O)c1ccc(NCC#Cc2sc3c(NC4CCC(N5CC6(COC6)C5)CC4)cccc3c2CC(F)(F)F)c(OCCO)c1 ; CNC(=O)c1cc(OCCO)c(NCC#Cc2sc3c(NC4CCN(C)CC4(F)F)cccc3c2CC(F)(F)F)cc1F ; CCN1CCC(Nc2cccc3c(CC(F)(F)F)c(C#CCNc4cc(F)c(C(=O)NC)cc4OCCO)sc23)CC1 ; CNC(=O)c1cc(OCCO)c(NCC#Cc2sc3c(NC4CCN(CC(O)CO)CC4F)cccc3c2CC(F)(F)F)cc1F]

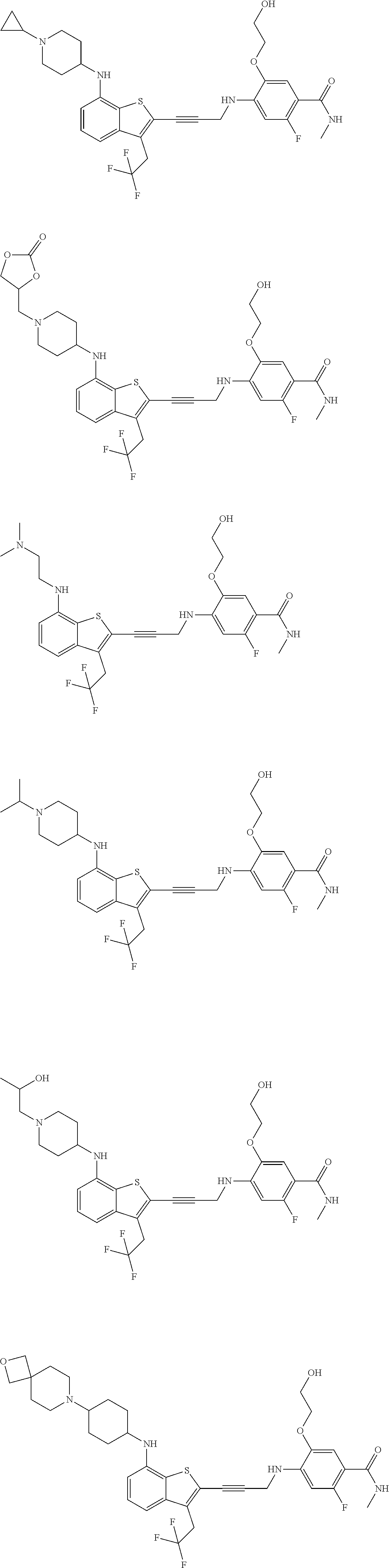 [SMILES: CNC(=O)c1cc(OCCO)c(NCC#Cc2sc3c(NC4CCN(C5CC5)CC4)cccc3c2CC(F)(F)F)cc1F ; CNC(=O)c1cc(OCCO)c(NCC#Cc2sc3c(NC4CCN(CC5COC(=O)O5)CC4)cccc3c2CC(F)(F)F)cc1F ; CNC(=O)c1cc(OCCO)c(NCC#Cc2sc3c(NCCN(C)C)cccc3c2CC(F)(F)F)cc1F ; CNC(=O)c1cc(OCCO)c(NCC#Cc2sc3c(NC4CCN(C(C)C)CC4)cccc3c2CC(F)(F)F)cc1F ; CNC(=O)c1cc(OCCO)c(NCC#Cc2sc3c(NC4CCN(CC(C)O)CC4)cccc3c2CC(F)(F)F)cc1F ; CNC(=O)c1cc(OCCO)c(NCC#Cc2sc3c(NC4CCC(N5CCC6(CC5)COC6)CC4)cccc3c2CC(F)(F)F)cc1F]

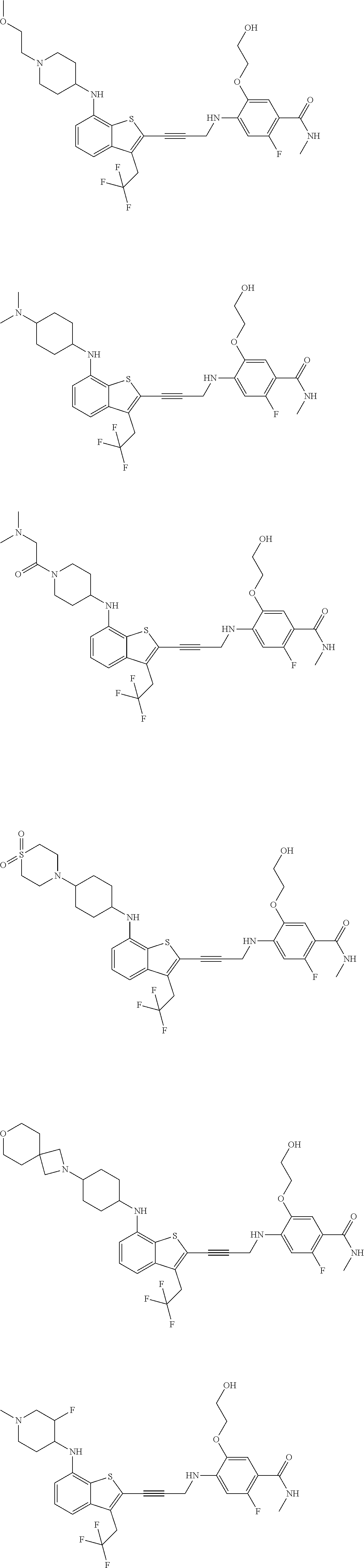 [SMILES: CNC(=O)c1cc(OCCO)c(NCC#Cc2sc3c(NC4CCN(CCOC)CC4)cccc3c2CC(F)(F)F)cc1F ; CNC(=O)c1cc(OCCO)c(NCC#Cc2sc3c(NC4CCC(N(C)C)CC4)cccc3c2CC(F)(F)F)cc1F ; CNC(=O)c1cc(OCCO)c(NCC#Cc2sc3c(NC4CCN(C(=O)CN(C)C)CC4)cccc3c2CC(F)(F)F)cc1F ; CNC(=O)c1cc(OCCO)c(NCC#Cc2sc3c(NC4CCC(N5CCS(=O)(=O)CC5)CC4)cccc3c2CC(F)(F)F)cc1F ; CNC(=O)c1cc(OCCO)c(NCC#Cc2sc3c(NC4CCC(N5CC6(CCOCC6)C5)CC4)cccc3c2CC(F)(F)F)cc1F ; CNC(=O)c1cc(OCCO)c(NCC#Cc2sc3c(NC4CCN(C)CC4F)cccc3c2CC(F)(F)F)cc1F]

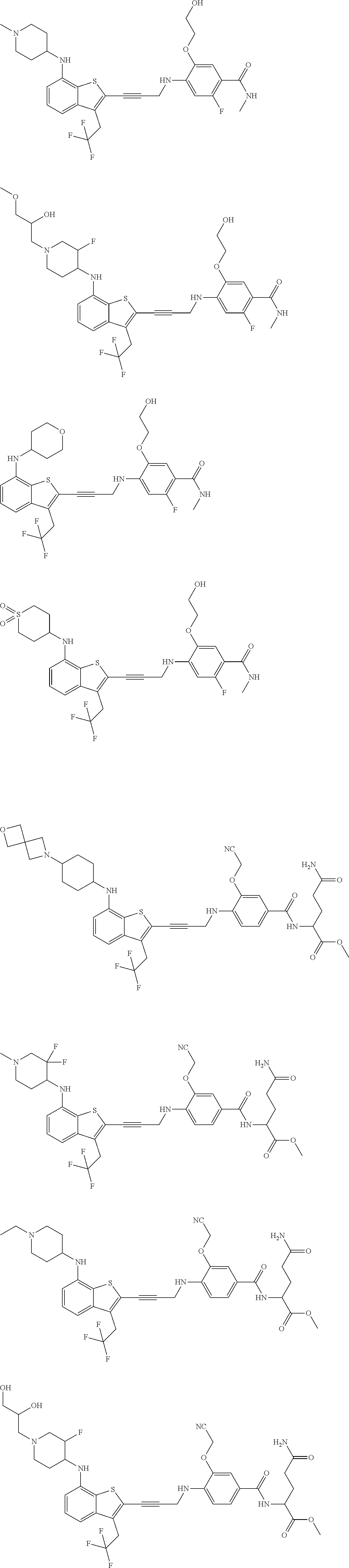

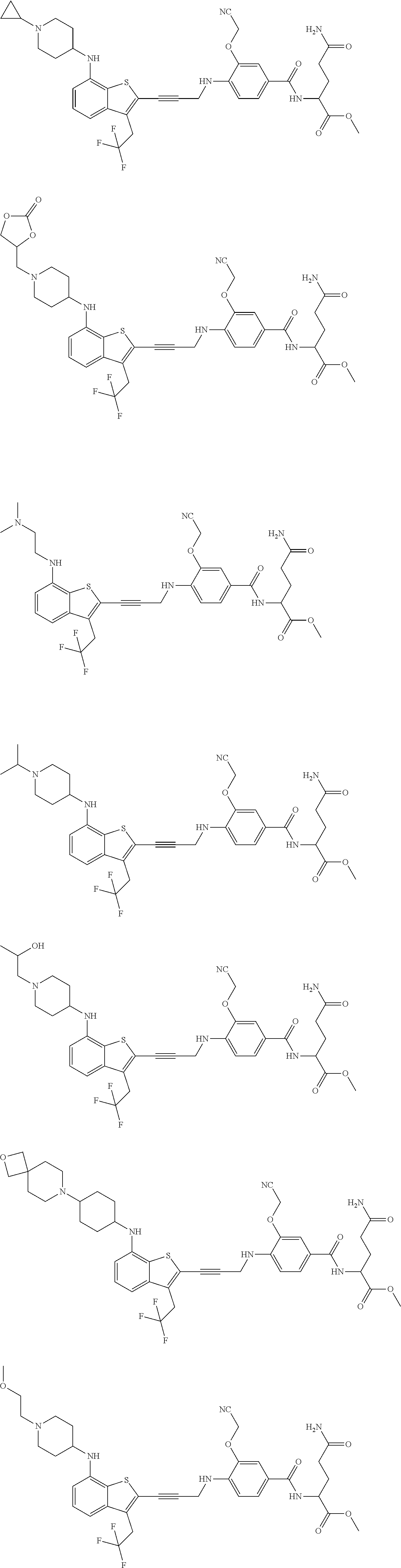

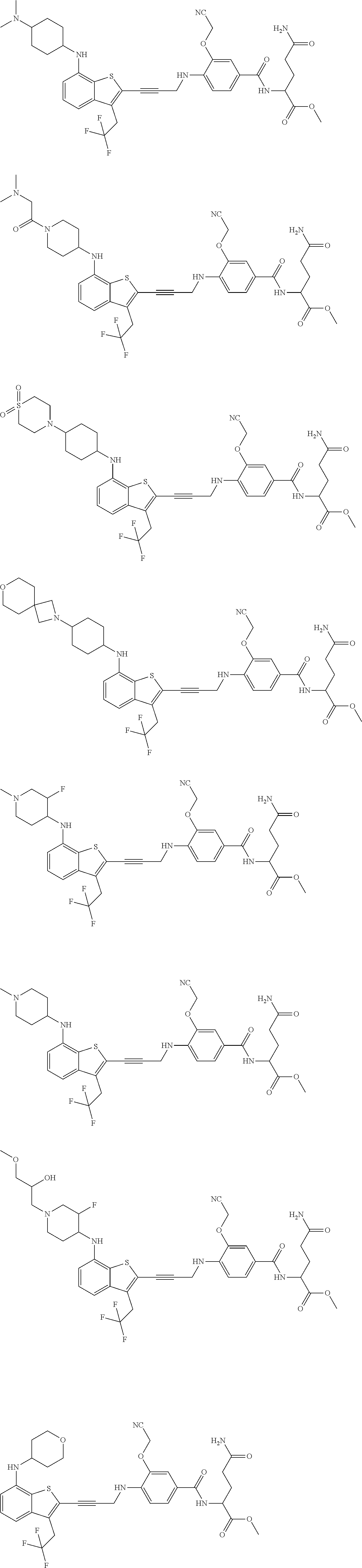

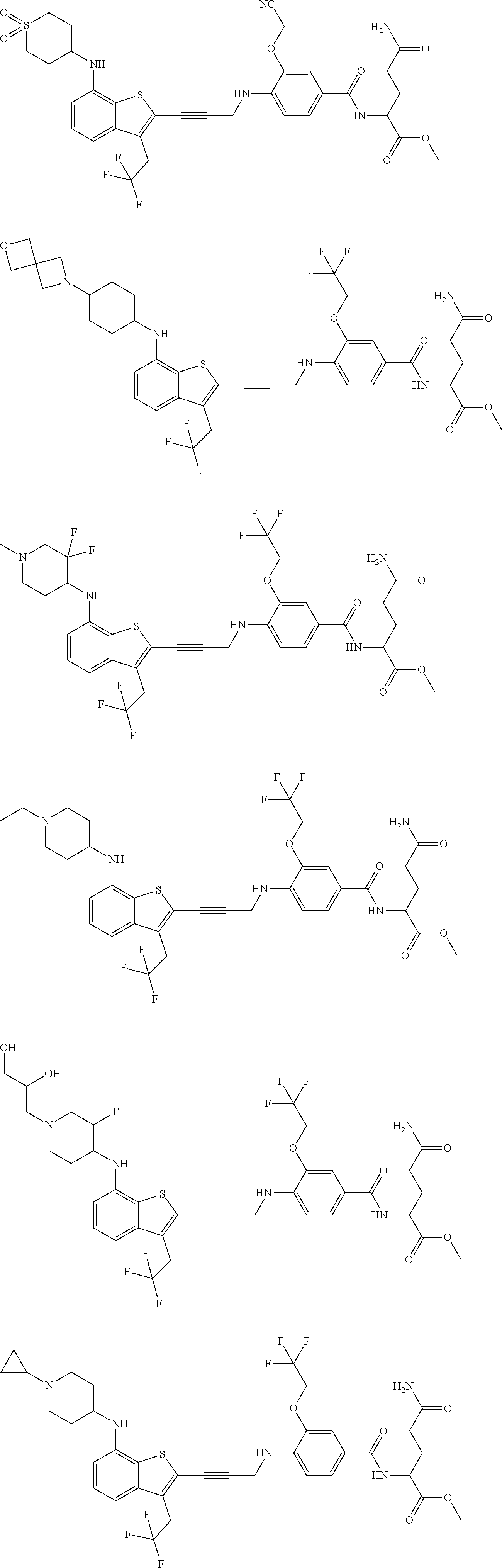

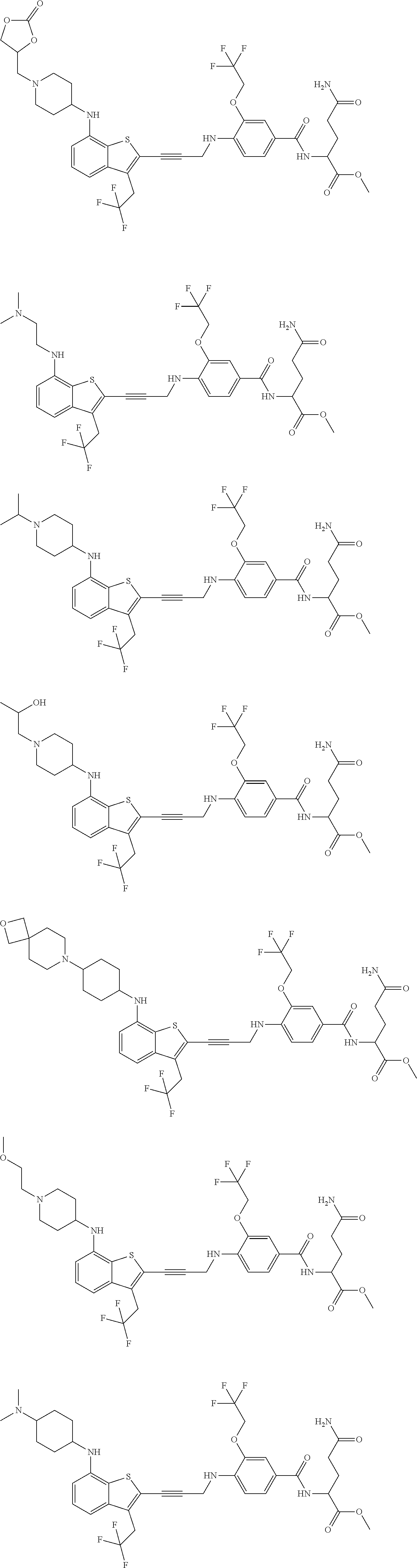

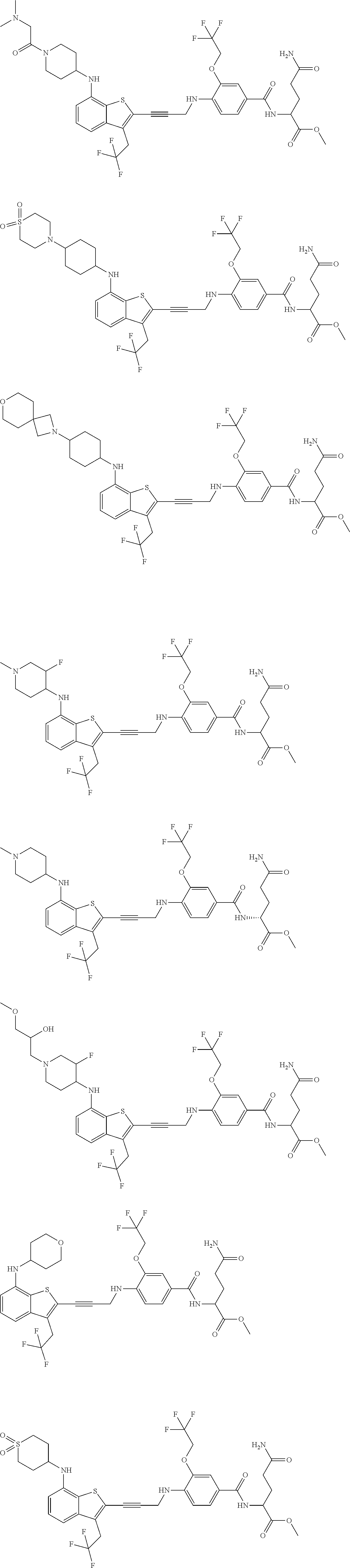

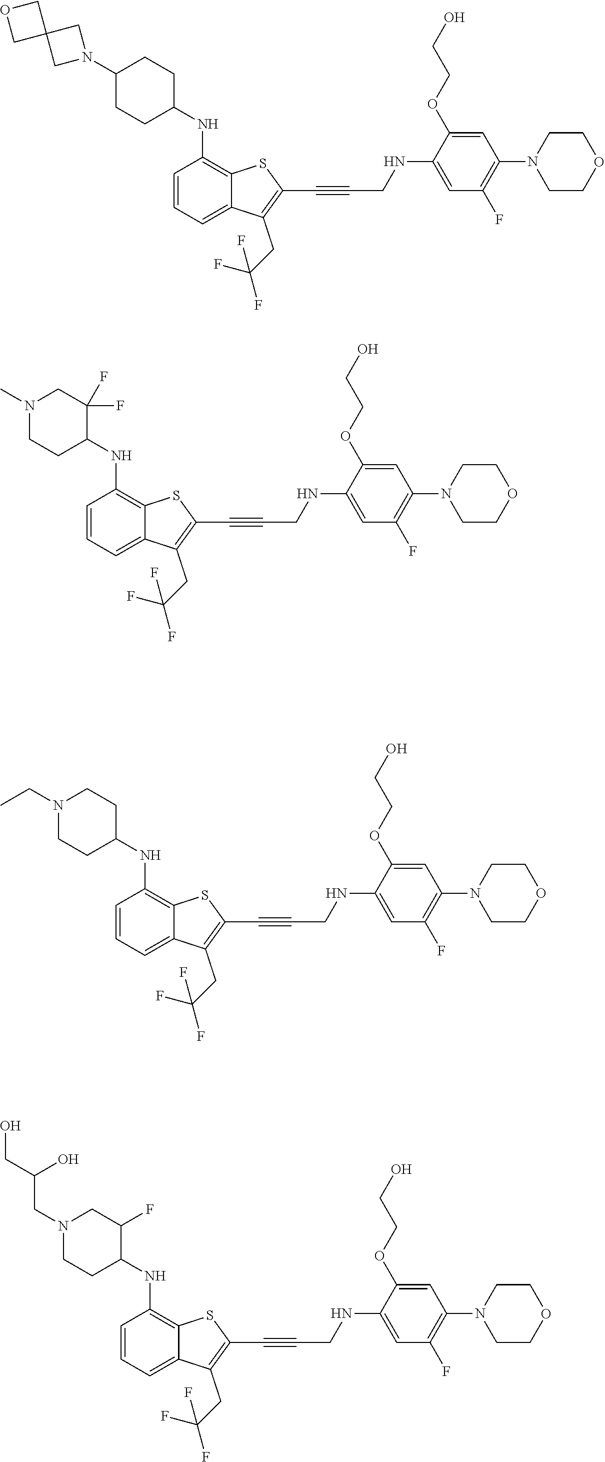 [SMILES: OCCOc1cc(N2CCOCC2)c(F)cc1NCC#Cc1sc2c(NC3CCC(N4CC5(COC5)C4)CC3)cccc2c1CC(F)(F)F ; CN1CCC(Nc2cccc3c(CC(F)(F)F)c(C#CCNc4cc(F)c(N5CCOCC5)cc4OCCO)sc23)C(F)(F)C1 ; CCN1CCC(Nc2cccc3c(CC(F)(F)F)c(C#CCNc4cc(F)c(N5CCOCC5)cc4OCCO)sc23)CC1 ; OCCOc1cc(N2CCOCC2)c(F)cc1NCC#Cc1sc2c(NC3CCN(CC(O)CO)CC3F)cccc2c1CC(F)(F)F]

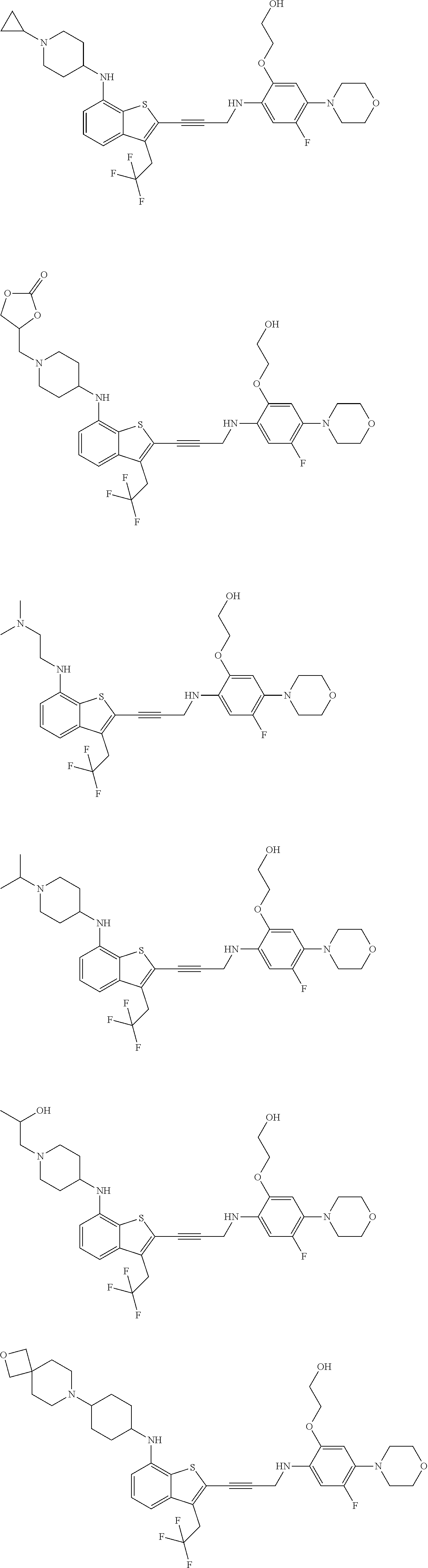 [SMILES: OCCOc1cc(N2CCOCC2)c(F)cc1NCC#Cc1sc2c(NC3CCN(C4CC4)CC3)cccc2c1CC(F)(F)F ; O=C1OCC(CN2CCC(Nc3cccc4c(CC(F)(F)F)c(C#CCNc5cc(F)c(N6CCOCC6)cc5OCCO)sc34)CC2)O1 ; CN(C)CCNc1cccc2c(CC(F)(F)F)c(C#CCNc3cc(F)c(N4CCOCC4)cc3OCCO)sc12 ; CC(C)N1CCC(Nc2cccc3c(CC(F)(F)F)c(C#CCNc4cc(F)c(N5CCOCC5)cc4OCCO)sc23)CC1 ; CC(O)CN1CCC(Nc2cccc3c(CC(F)(F)F)c(C#CCNc4cc(F)c(N5CCOCC5)cc4OCCO)sc23)CC1 ; OCCOc1cc(N2CCOCC2)c(F)cc1NCC#Cc1sc2c(NC3CCC(N4CCC5(CC4)COC5)CC3)cccc2c1CC(F)(F)F]

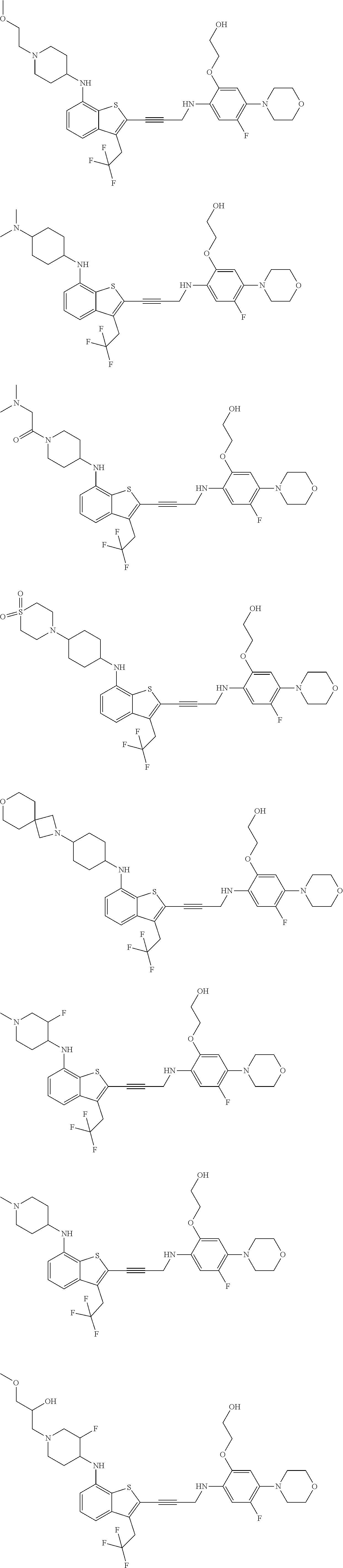

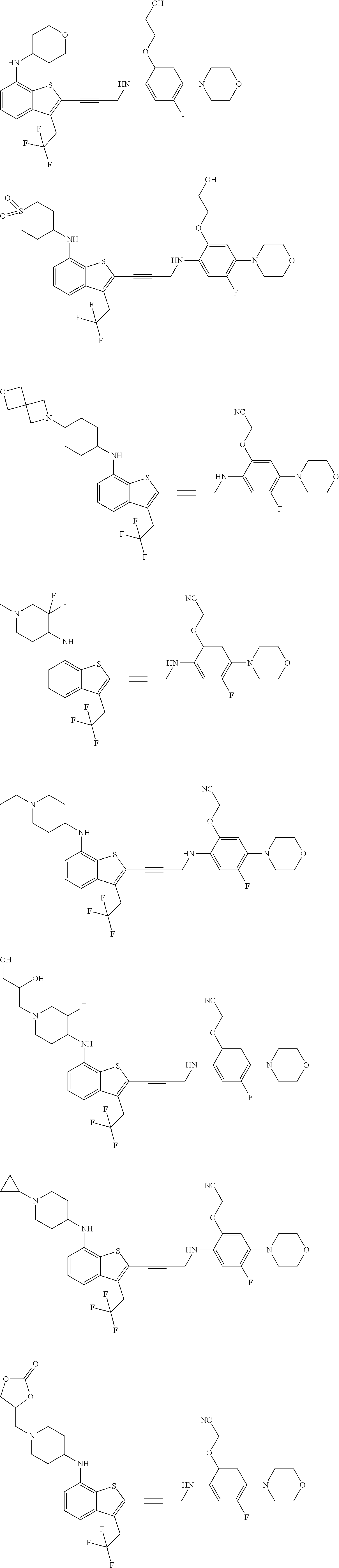

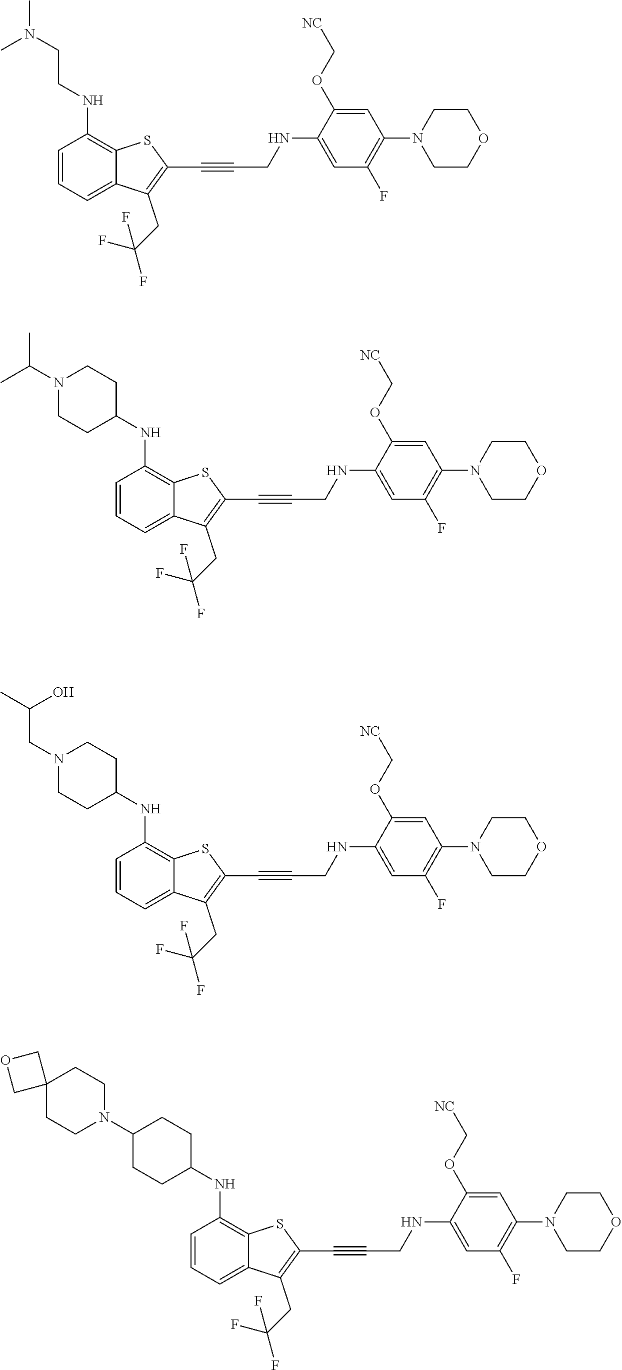 [SMILES: CN(C)CCNc1cccc2c(CC(F)(F)F)c(C#CCNc3cc(F)c(N4CCOCC4)cc3OCC#N)sc12 ; CC(C)N1CCC(Nc2cccc3c(CC(F)(F)F)c(C#CCNc4cc(F)c(N5CCOCC5)cc4OCC#N)sc23)CC1 ; CC(O)CN1CCC(Nc2cccc3c(CC(F)(F)F)c(C#CCNc4cc(F)c(N5CCOCC5)cc4OCC#N)sc23)CC1 ; N#CCOc1cc(N2CCOCC2)c(F)cc1NCC#Cc1sc2c(NC3CCC(N4CCC5(CC4)COC5)CC3)cccc2c1CC(F)(F)F]

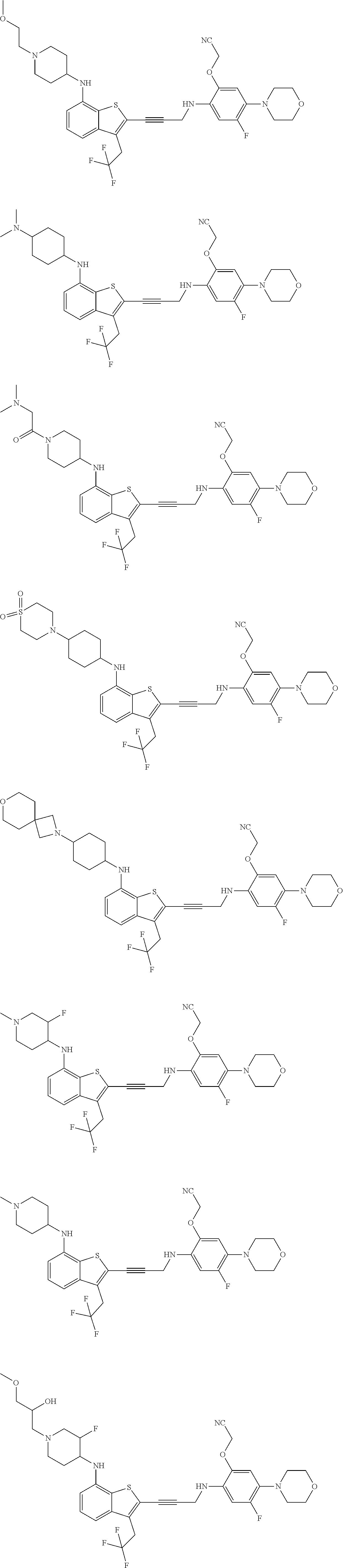

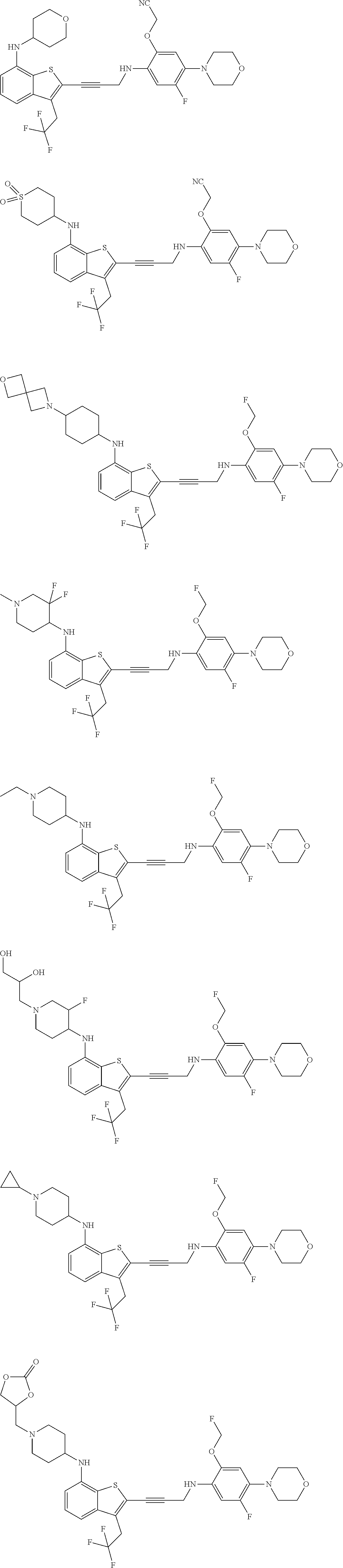

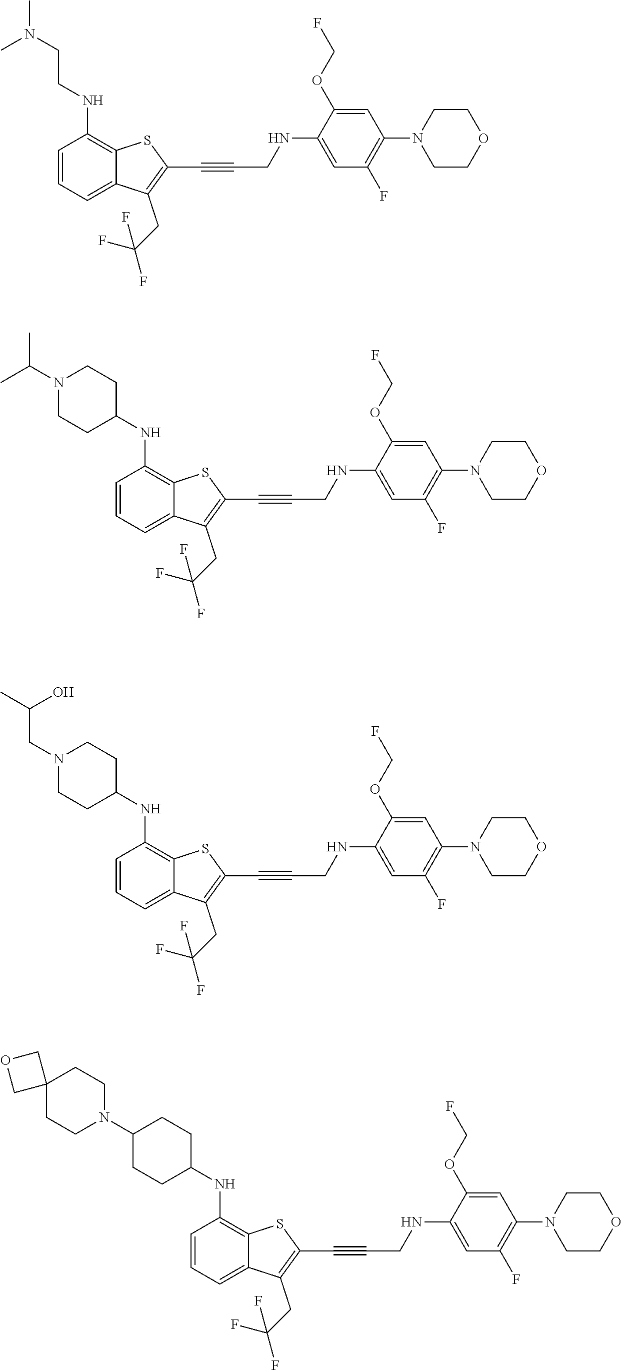 [SMILES: CN(C)CCNc1cccc2c(CC(F)(F)F)c(C#CCNc3cc(F)c(N4CCOCC4)cc3OCF)sc12 ; CC(C)N1CCC(Nc2cccc3c(CC(F)(F)F)c(C#CCNc4cc(F)c(N5CCOCC5)cc4OCF)sc23)CC1 ; CC(O)CN1CCC(Nc2cccc3c(CC(F)(F)F)c(C#CCNc4cc(F)c(N5CCOCC5)cc4OCF)sc23)CC1 ; FCOc1cc(N2CCOCC2)c(F)cc1NCC#Cc1sc2c(NC3CCC(N4CCC5(CC4)COC5)CC3)cccc2c1CC(F)(F)F]

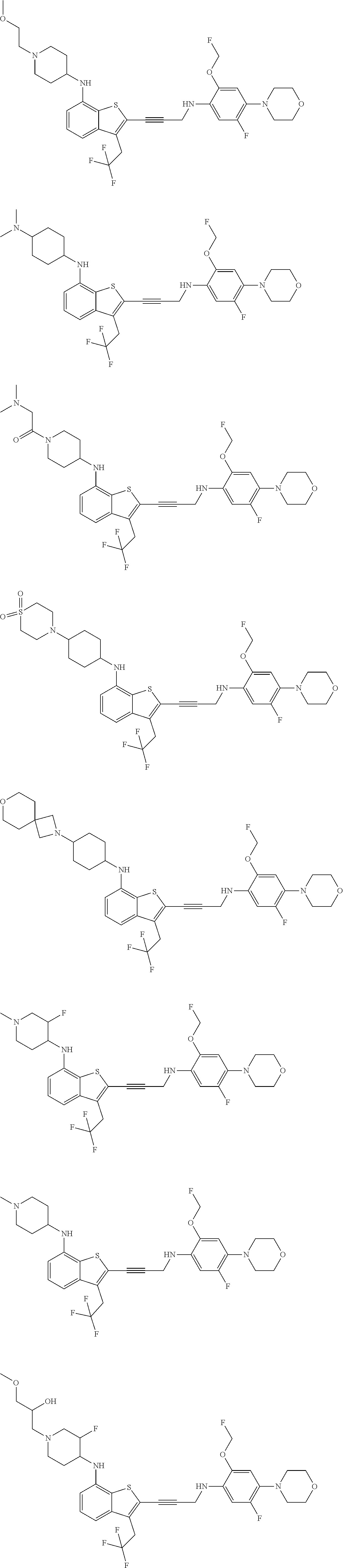

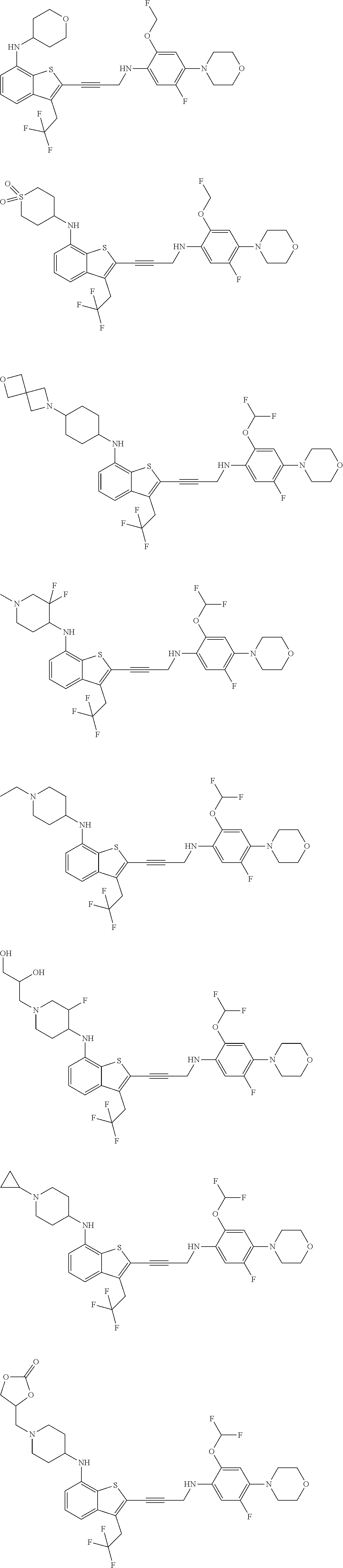

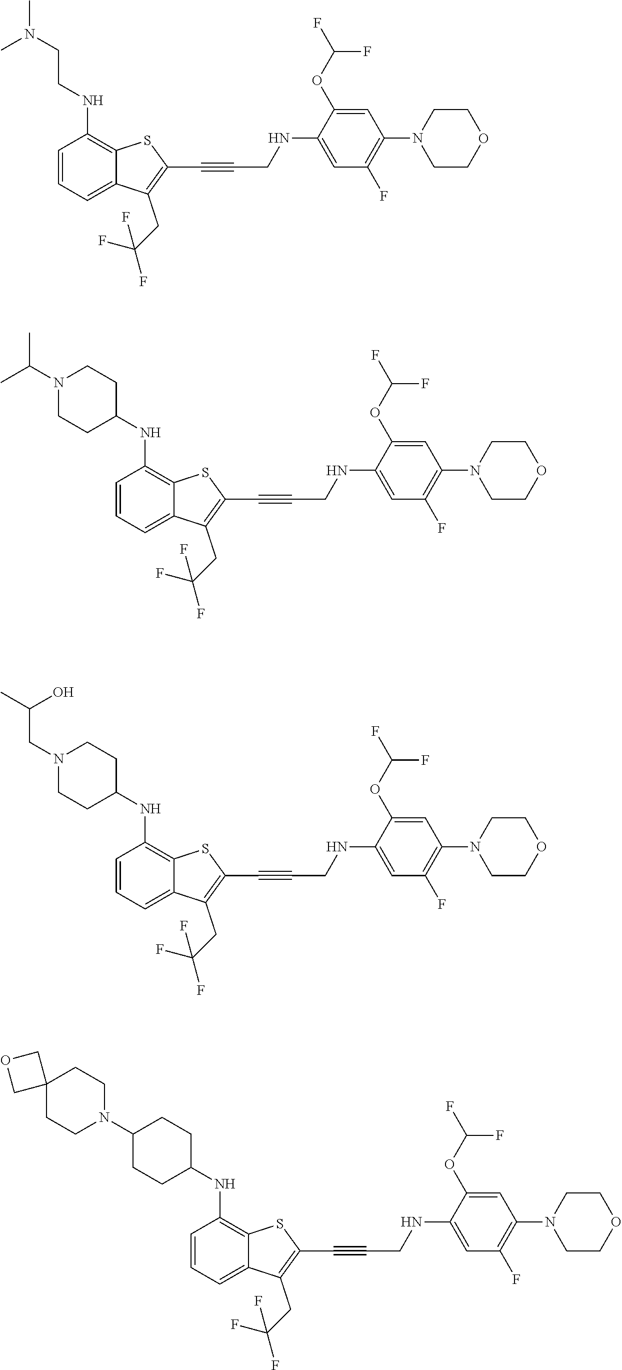 [SMILES: CN(C)CCNc1cccc2c(CC(F)(F)F)c(C#CCNc3cc(F)c(N4CCOCC4)cc3OC(F)F)sc12 ; CC(C)N1CCC(Nc2cccc3c(CC(F)(F)F)c(C#CCNc4cc(F)c(N5CCOCC5)cc4OC(F)F)sc23)CC1 ; CC(O)CN1CCC(Nc2cccc3c(CC(F)(F)F)c(C#CCNc4cc(F)c(N5CCOCC5)cc4OC(F)F)sc23)CC1 ; Fc1cc(NCC#Cc2sc3c(NC4CCC(N5CCC6(CC5)COC6)CC4)cccc3c2CC(F)(F)F)c(OC(F)F)cc1N1CCOCC1]

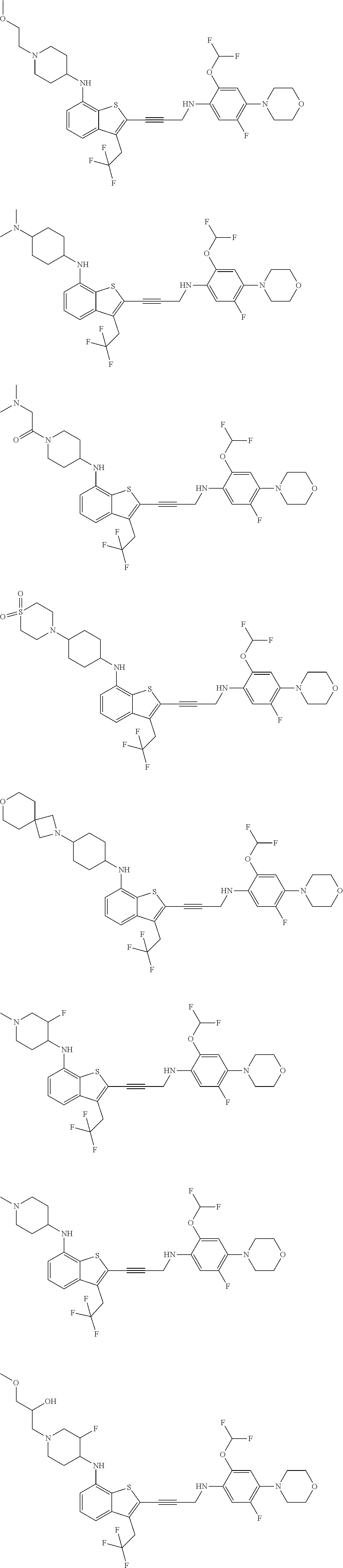

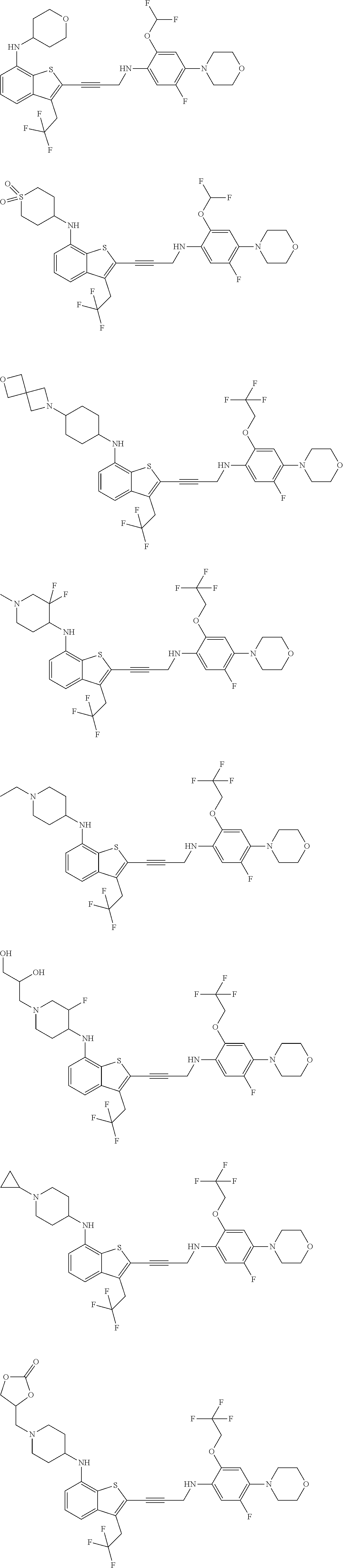

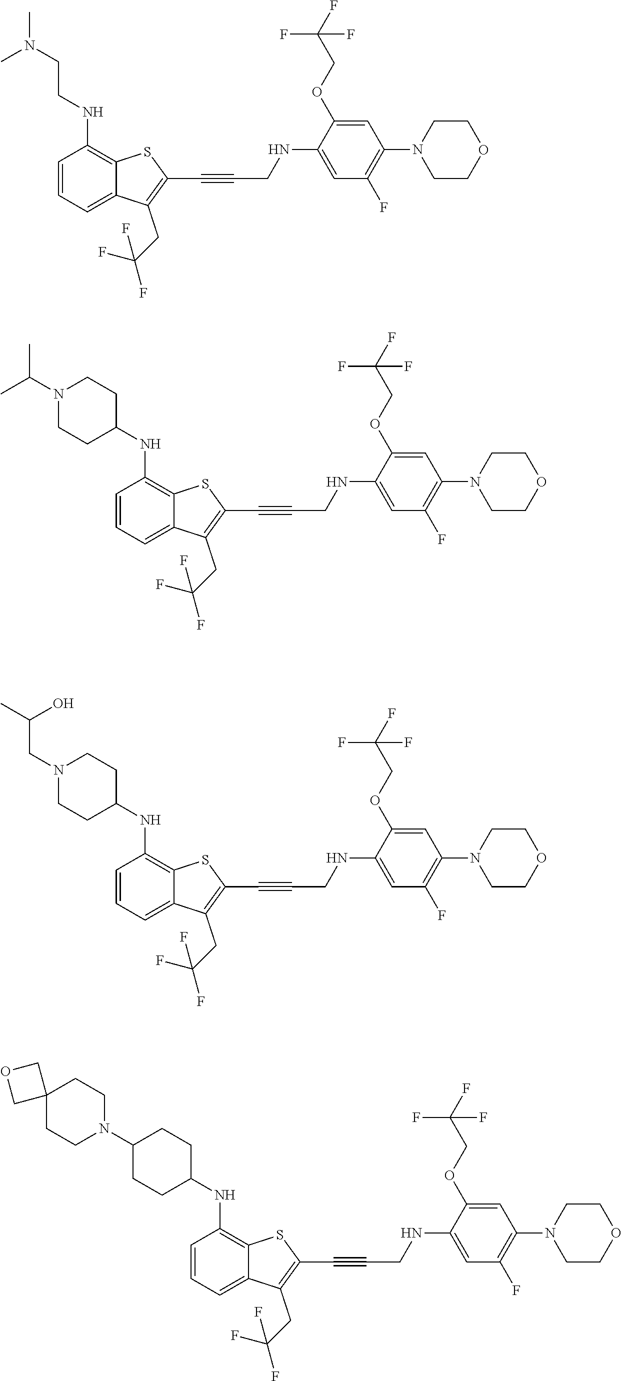 [SMILES: CN(C)CCNc1cccc2c(CC(F)(F)F)c(C#CCNc3cc(F)c(N4CCOCC4)cc3OCC(F)(F)F)sc12 ; CC(C)N1CCC(Nc2cccc3c(CC(F)(F)F)c(C#CCNc4cc(F)c(N5CCOCC5)cc4OCC(F)(F)F)sc23)CC1 ; CC(O)CN1CCC(Nc2cccc3c(CC(F)(F)F)c(C#CCNc4cc(F)c(N5CCOCC5)cc4OCC(F)(F)F)sc23)CC1 ; Fc1cc(NCC#Cc2sc3c(NC4CCC(N5CCC6(CC5)COC6)CC4)cccc3c2CC(F)(F)F)c(OCC(F)(F)F)cc1N1CCOCC1]

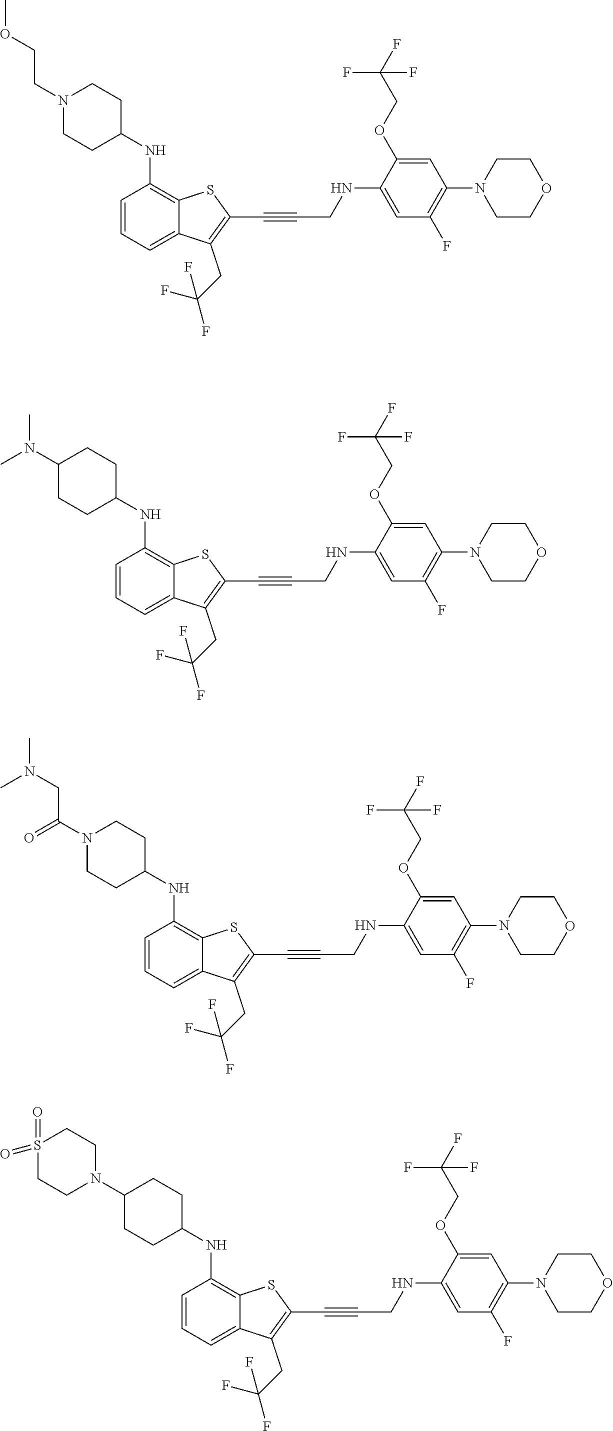 [SMILES: COCCN1CCC(Nc2cccc3c(CC(F)(F)F)c(C#CCNc4cc(F)c(N5CCOCC5)cc4OCC(F)(F)F)sc23)CC1 ; CN(C)C1CCC(Nc2cccc3c(CC(F)(F)F)c(C#CCNc4cc(F)c(N5CCOCC5)cc4OCC(F)(F)F)sc23)CC1 ; CN(C)CC(=O)N1CCC(Nc2cccc3c(CC(F)(F)F)c(C#CCNc4cc(F)c(N5CCOCC5)cc4OCC(F)(F)F)sc23)CC1 ; O=S1(=O)CCN(C2CCC(Nc3cccc4c(CC(F)(F)F)c(C#CCNc5cc(F)c(N6CCOCC6)cc5OCC(F)(F)F)sc34)CC2)CC1]

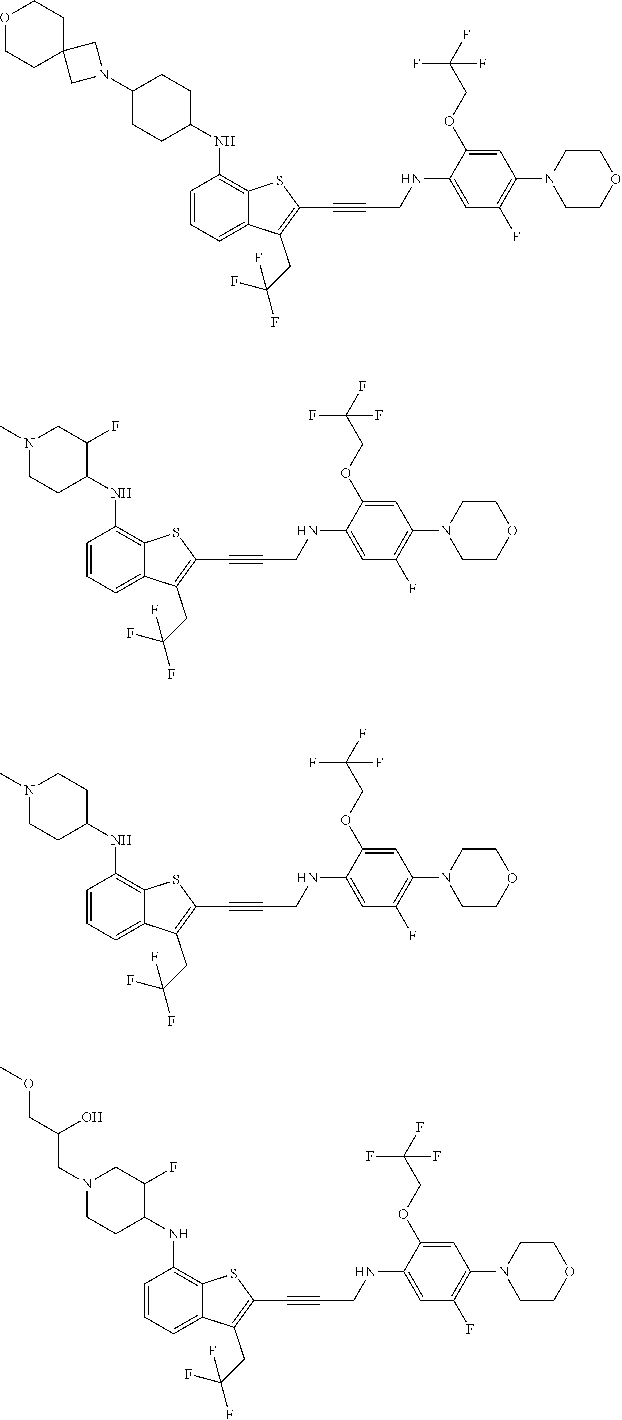 [SMILES: Fc1cc(NCC#Cc2sc3c(NC4CCC(N5CC6(CCOCC6)C5)CC4)cccc3c2CC(F)(F)F)c(OCC(F)(F)F)cc1N1CCOCC1 ; CN1CCC(Nc2cccc3c(CC(F)(F)F)c(C#CCNc4cc(F)c(N5CCOCC5)cc4OCC(F)(F)F)sc23)C(F)C1 ; CN1CCC(Nc2cccc3c(CC(F)(F)F)c(C#CCNc4cc(F)c(N5CCOCC5)cc4OCC(F)(F)F)sc23)CC1 ; COCC(O)CN1CCC(Nc2cccc3c(CC(F)(F)F)c(C#CCNc4cc(F)c(N5CCOCC5)cc4OCC(F)(F)F)sc23)C(F)C1]

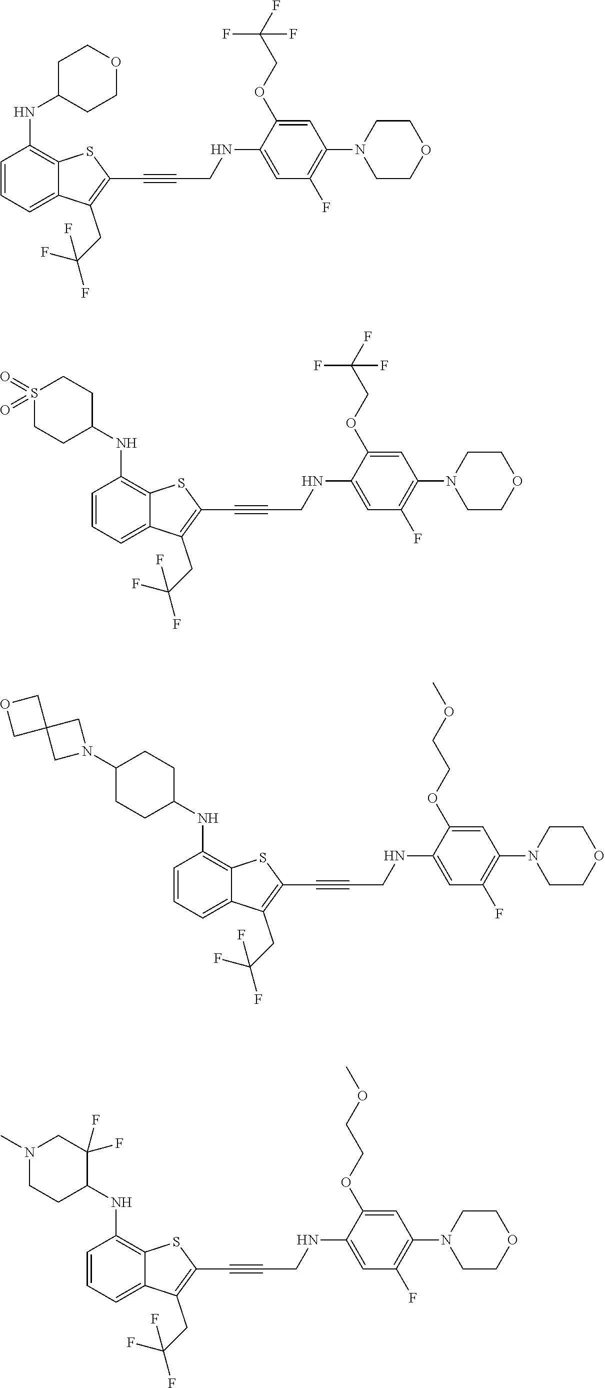 [SMILES: Fc1cc(NCC#Cc2sc3c(NC4CCOCC4)cccc3c2CC(F)(F)F)c(OCC(F)(F)F)cc1N1CCOCC1 ; O=S1(=O)CCC(Nc2cccc3c(CC(F)(F)F)c(C#CCNc4cc(F)c(N5CCOCC5)cc4OCC(F)(F)F)sc23)CC1 ; COCCOc1cc(N2CCOCC2)c(F)cc1NCC#Cc1sc2c(NC3CCC(N4CC5(COC5)C4)CC3)cccc2c1CC(F)(F)F ; COCCOc1cc(N2CCOCC2)c(F)cc1NCC#Cc1sc2c(NC3CCN(C)CC3(F)F)cccc2c1CC(F)(F)F]

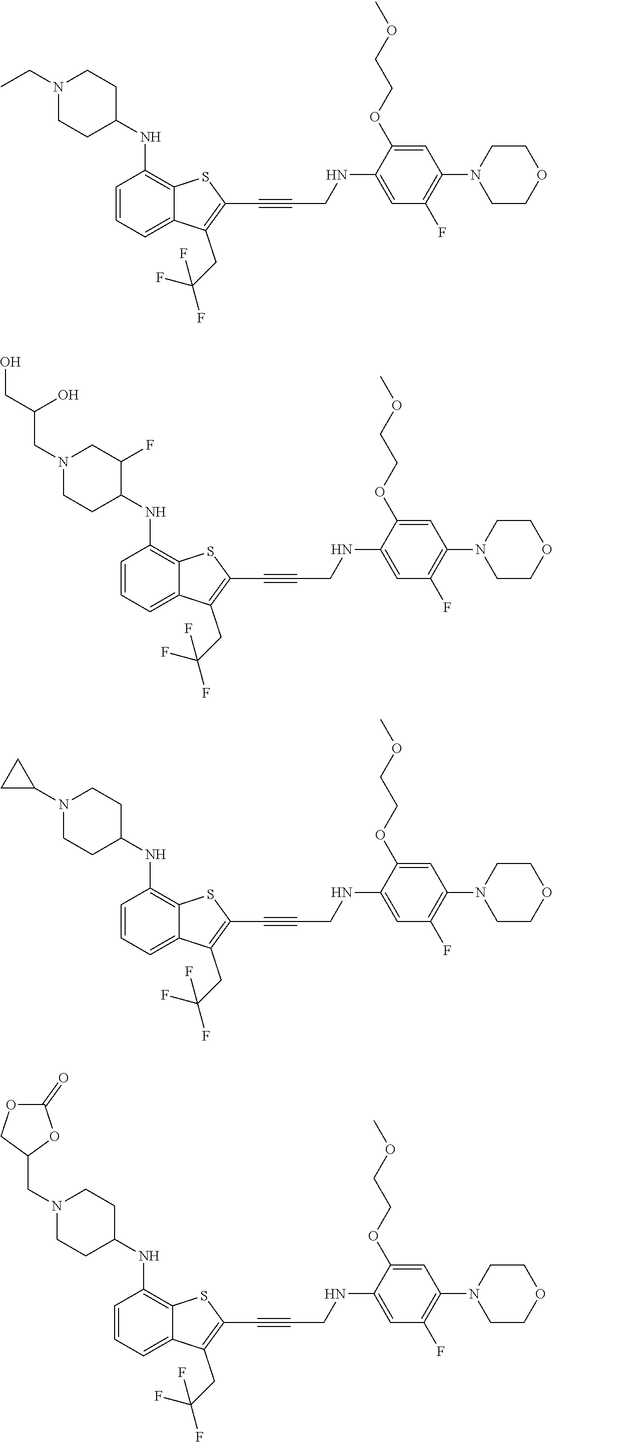 [SMILES: CCN1CCC(Nc2cccc3c(CC(F)(F)F)c(C#CCNc4cc(F)c(N5CCOCC5)cc4OCCOC)sc23)CC1 ; COCCOc1cc(N2CCOCC2)c(F)cc1NCC#Cc1sc2c(NC3CCN(CC(O)CO)CC3F)cccc2c1CC(F)(F)F ; COCCOc1cc(N2CCOCC2)c(F)cc1NCC#Cc1sc2c(NC3CCN(C4CC4)CC3)cccc2c1CC(F)(F)F ; COCCOc1cc(N2CCOCC2)c(F)cc1NCC#Cc1sc2c(NC3CCN(CC4COC(=O)O4)CC3)cccc2c1CC(F)(F)F]

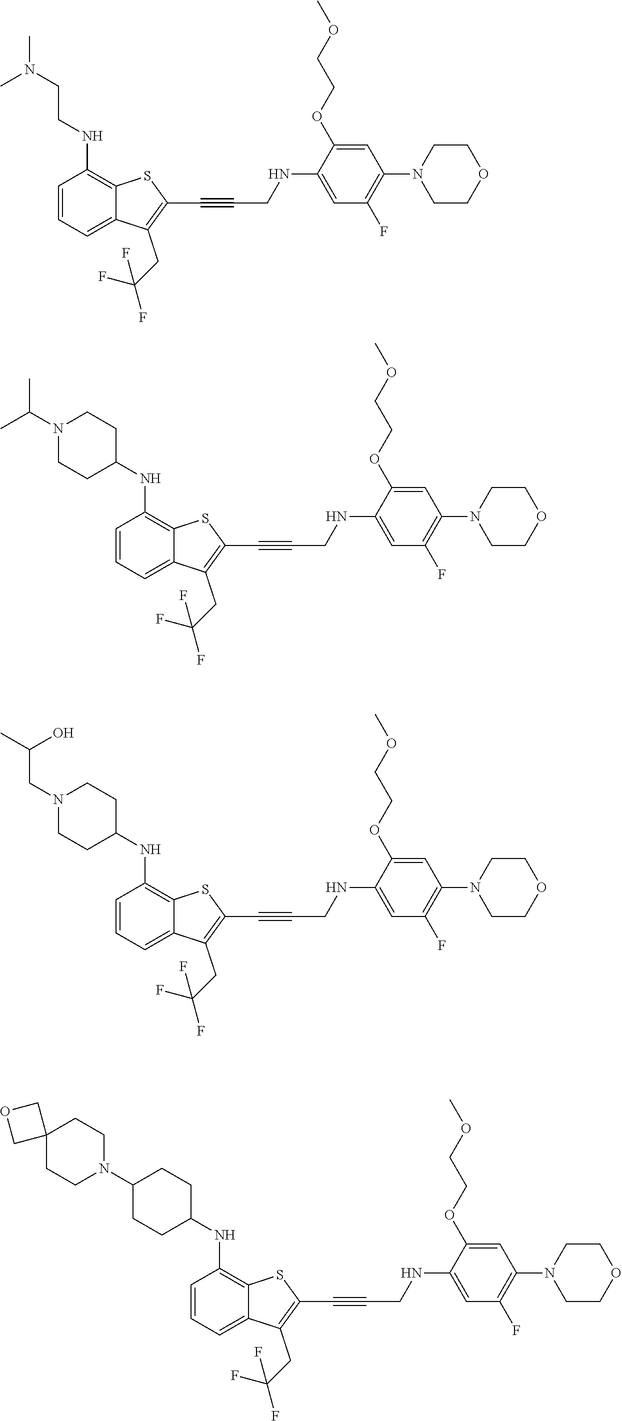 [SMILES: COCCOc1cc(N2CCOCC2)c(F)cc1NCC#Cc1sc2c(NCCN(C)C)cccc2c1CC(F)(F)F ; COCCOc1cc(N2CCOCC2)c(F)cc1NCC#Cc1sc2c(NC3CCN(C(C)C)CC3)cccc2c1CC(F)(F)F ; COCCOc1cc(N2CCOCC2)c(F)cc1NCC#Cc1sc2c(NC3CCN(CC(C)O)CC3)cccc2c1CC(F)(F)F ; COCCOc1cc(N2CCOCC2)c(F)cc1NCC#Cc1sc2c(NC3CCC(N4CCC5(CC4)COC5)CC3)cccc2c1CC(F)(F)F]

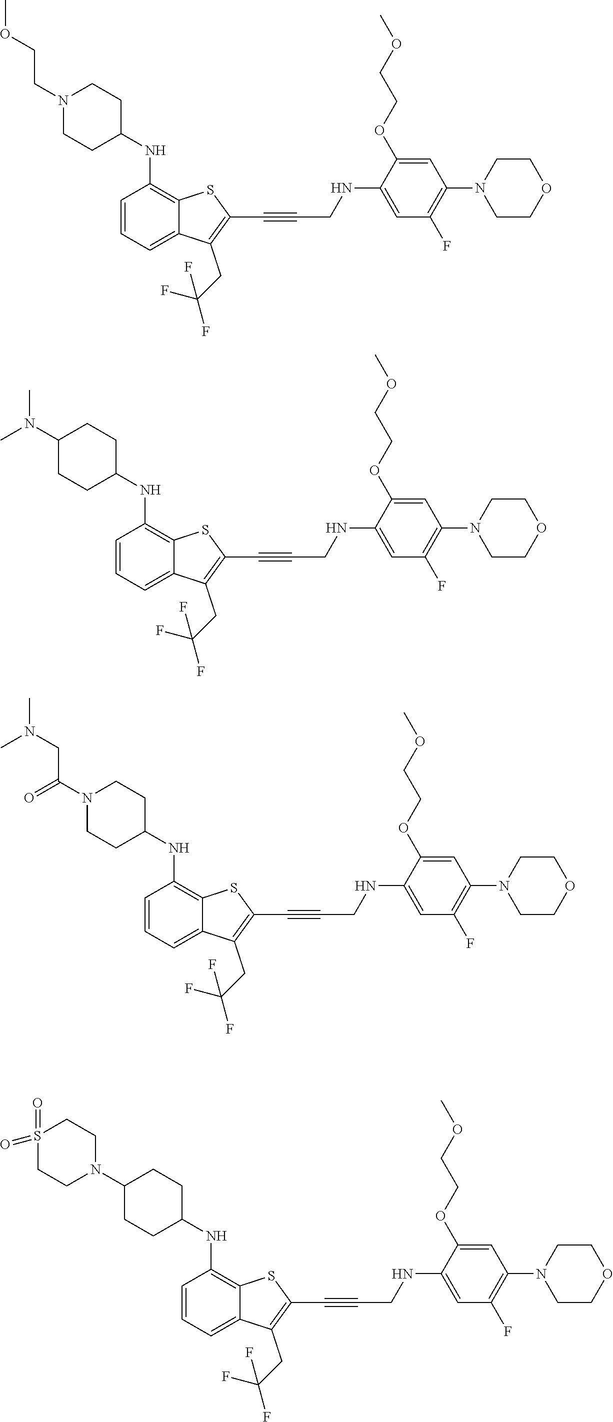 [SMILES: COCCOc1cc(N2CCOCC2)c(F)cc1NCC#Cc1sc2c(NC3CCN(CCOC)CC3)cccc2c1CC(F)(F)F ; COCCOc1cc(N2CCOCC2)c(F)cc1NCC#Cc1sc2c(NC3CCC(N(C)C)CC3)cccc2c1CC(F)(F)F ; COCCOc1cc(N2CCOCC2)c(F)cc1NCC#Cc1sc2c(NC3CCN(C(=O)CN(C)C)CC3)cccc2c1CC(F)(F)F ; COCCOc1cc(N2CCOCC2)c(F)cc1NCC#Cc1sc2c(NC3CCC(N4CCS(=O)(=O)CC4)CC3)cccc2c1CC(F)(F)F]

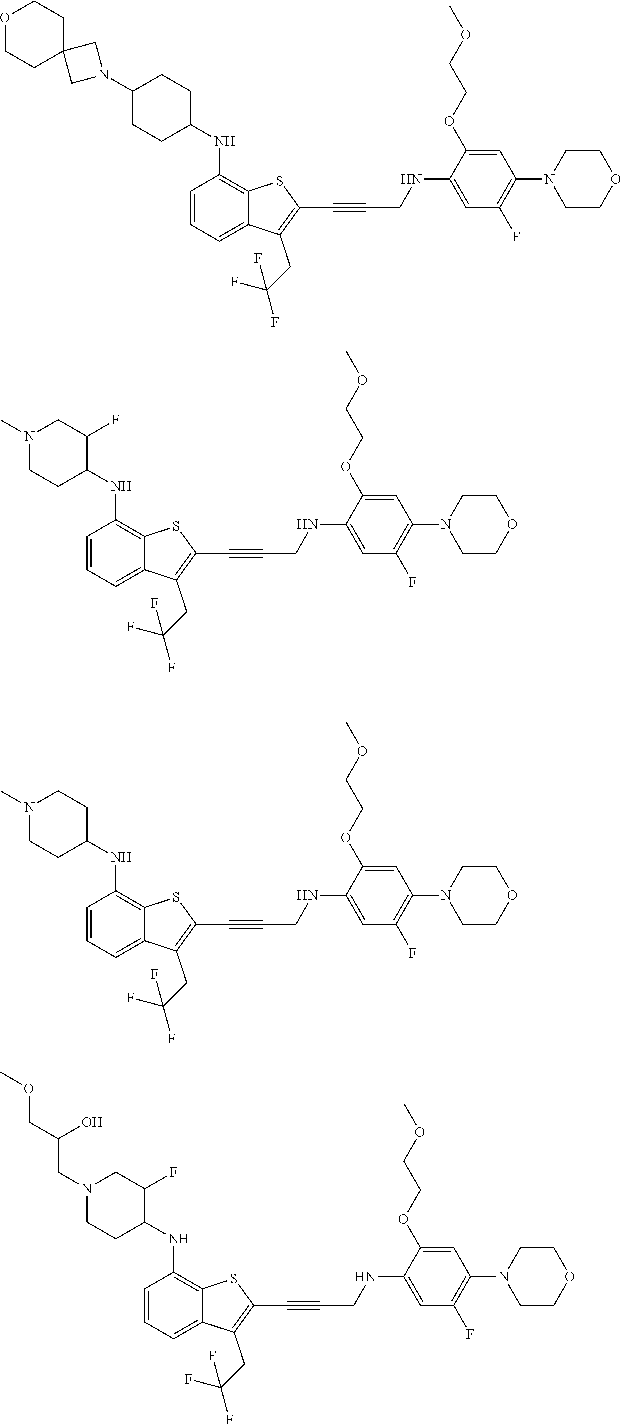 [SMILES: COCCOc1cc(N2CCOCC2)c(F)cc1NCC#Cc1sc2c(NC3CCC(N4CC5(CCOCC5)C4)CC3)cccc2c1CC(F)(F)F ; COCCOc1cc(N2CCOCC2)c(F)cc1NCC#Cc1sc2c(NC3CCN(C)CC3F)cccc2c1CC(F)(F)F ; COCCOc1cc(N2CCOCC2)c(F)cc1NCC#Cc1sc2c(NC3CCN(C)CC3)cccc2c1CC(F)(F)F ; COCCOc1cc(N2CCOCC2)c(F)cc1NCC#Cc1sc2c(NC3CCN(CC(O)COC)CC3F)cccc2c1CC(F)(F)F]

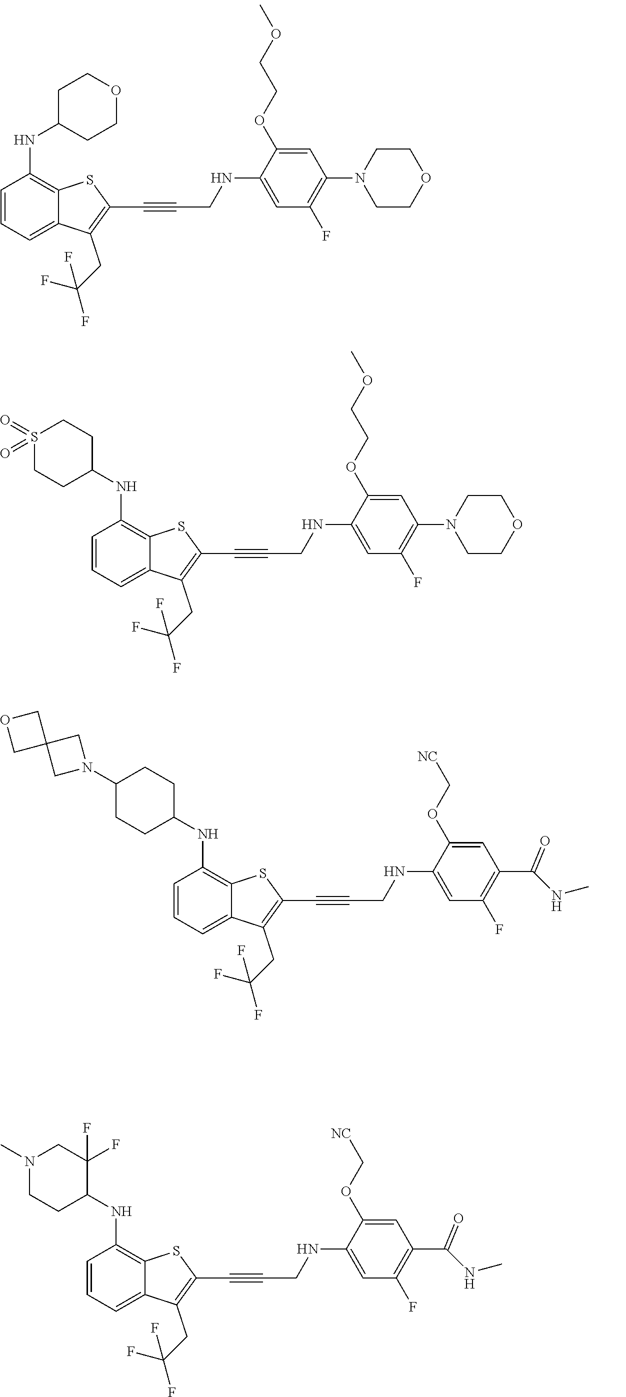

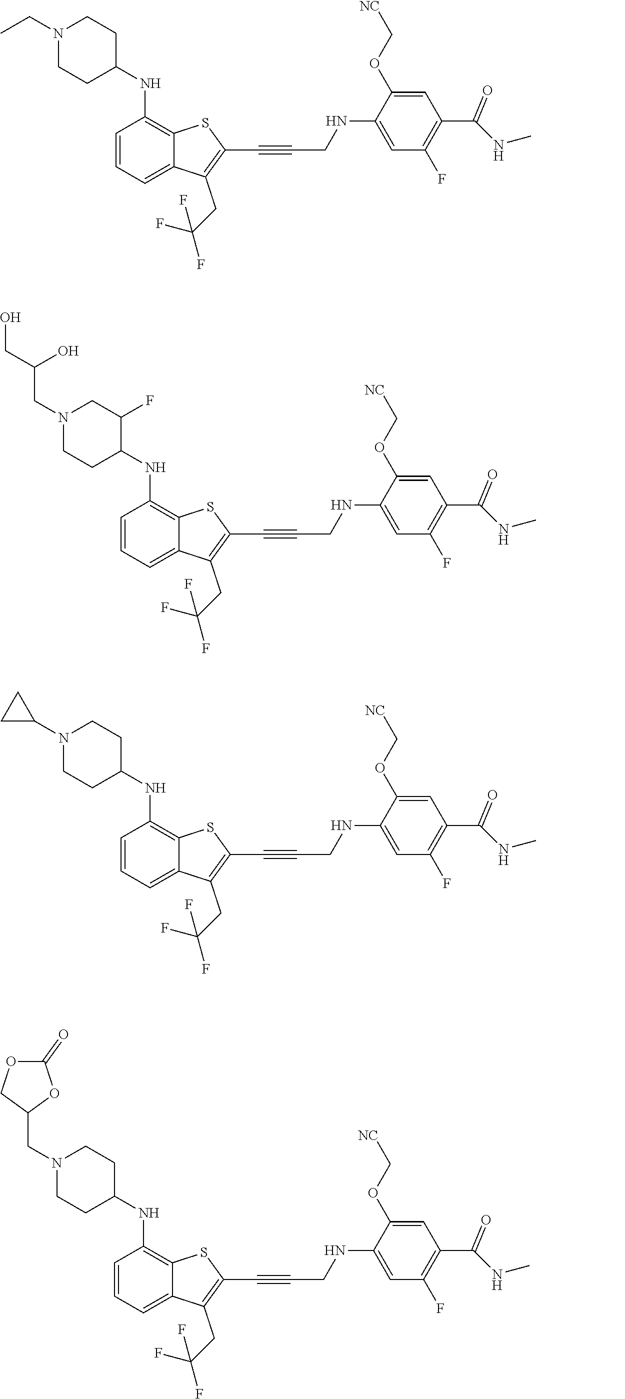 [SMILES: CCN1CCC(Nc2cccc3c(CC(F)(F)F)c(C#CCNc4cc(F)c(C(=O)NC)cc4OCC#N)sc23)CC1 ; CNC(=O)c1cc(OCC#N)c(NCC#Cc2sc3c(NC4CCN(CC(O)CO)CC4F)cccc3c2CC(F)(F)F)cc1F ; CNC(=O)c1cc(OCC#N)c(NCC#Cc2sc3c(NC4CCN(C5CC5)CC4)cccc3c2CC(F)(F)F)cc1F ; CNC(=O)c1cc(OCC#N)c(NCC#Cc2sc3c(NC4CCN(CC5COC(=O)O5)CC4)cccc3c2CC(F)(F)F)cc1F]

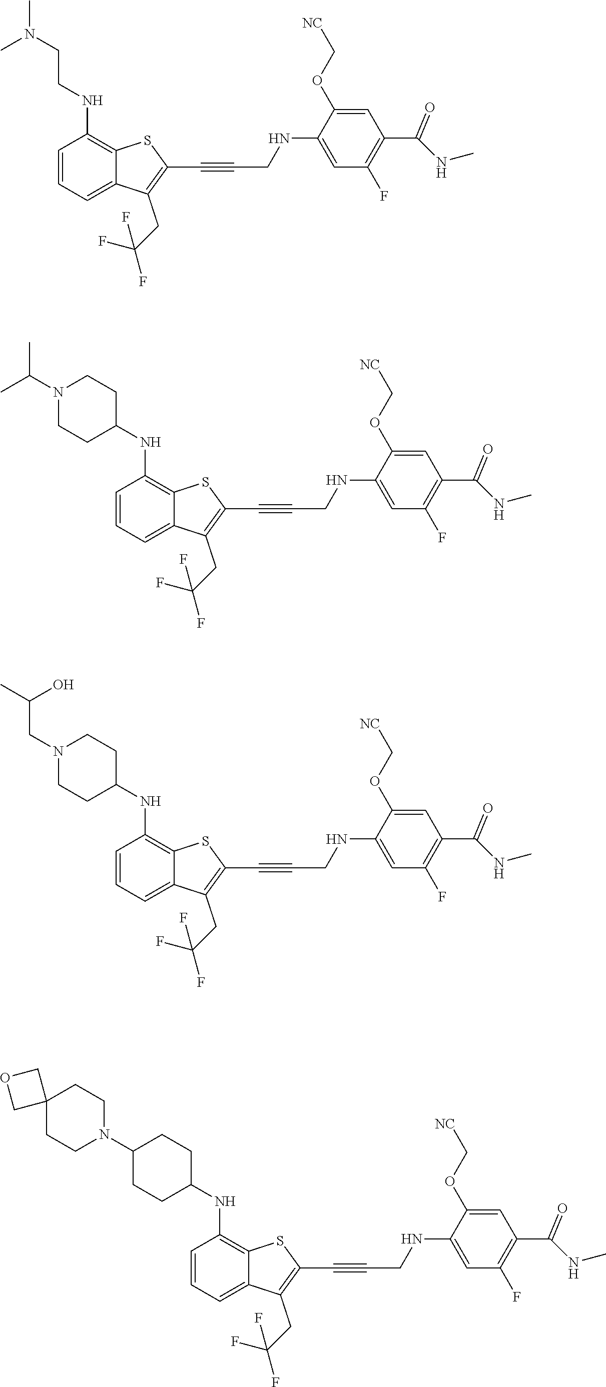

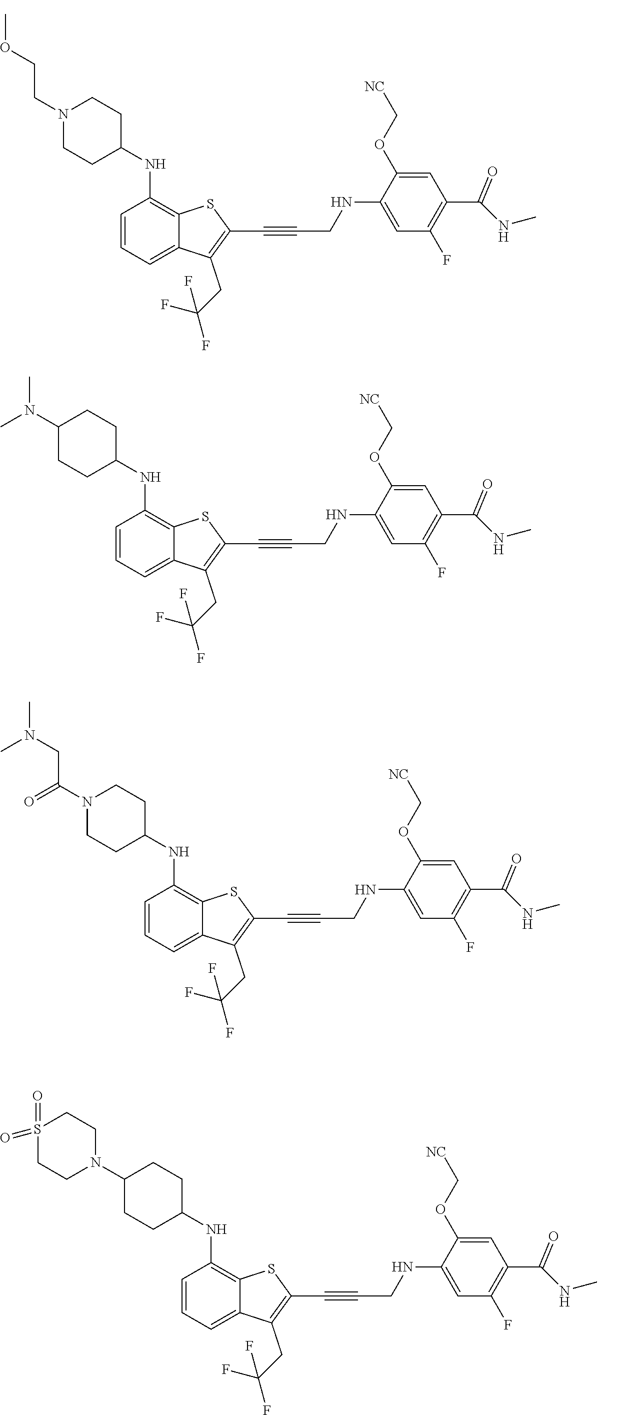

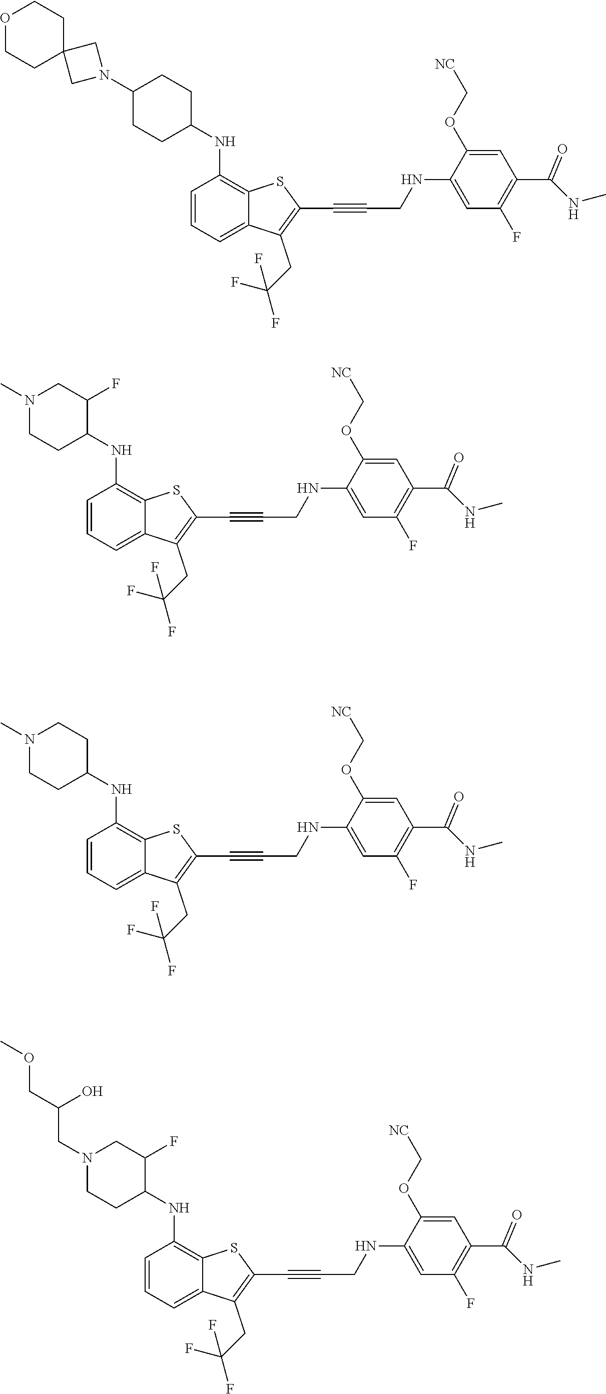

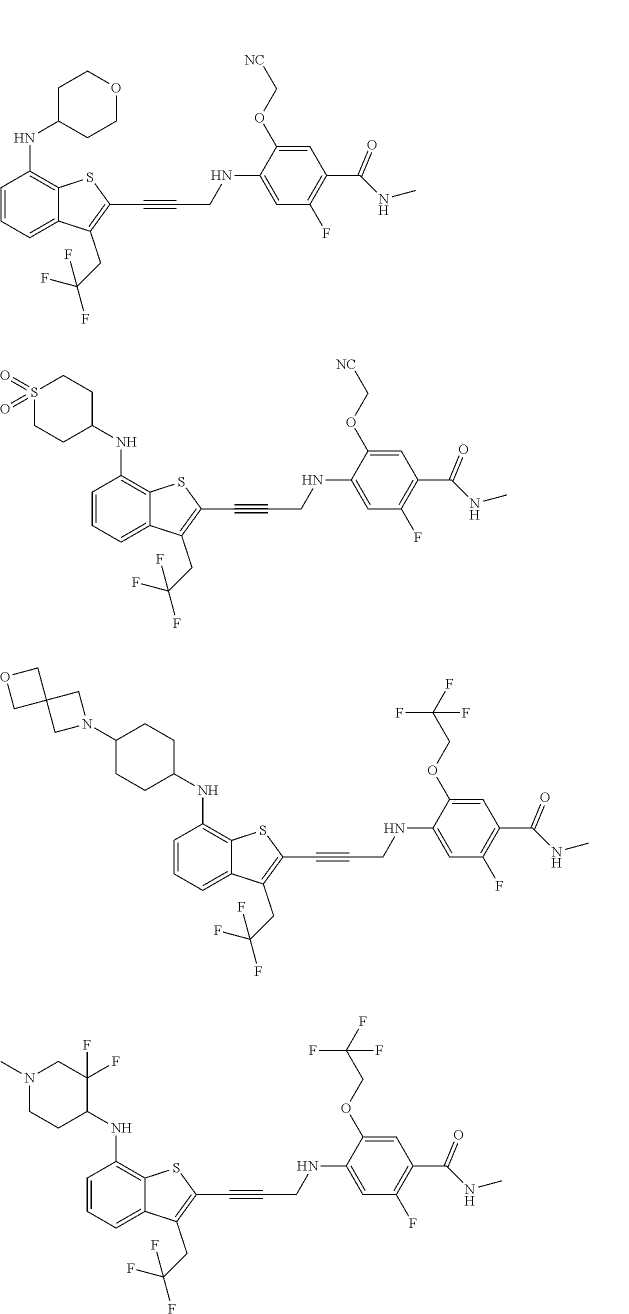

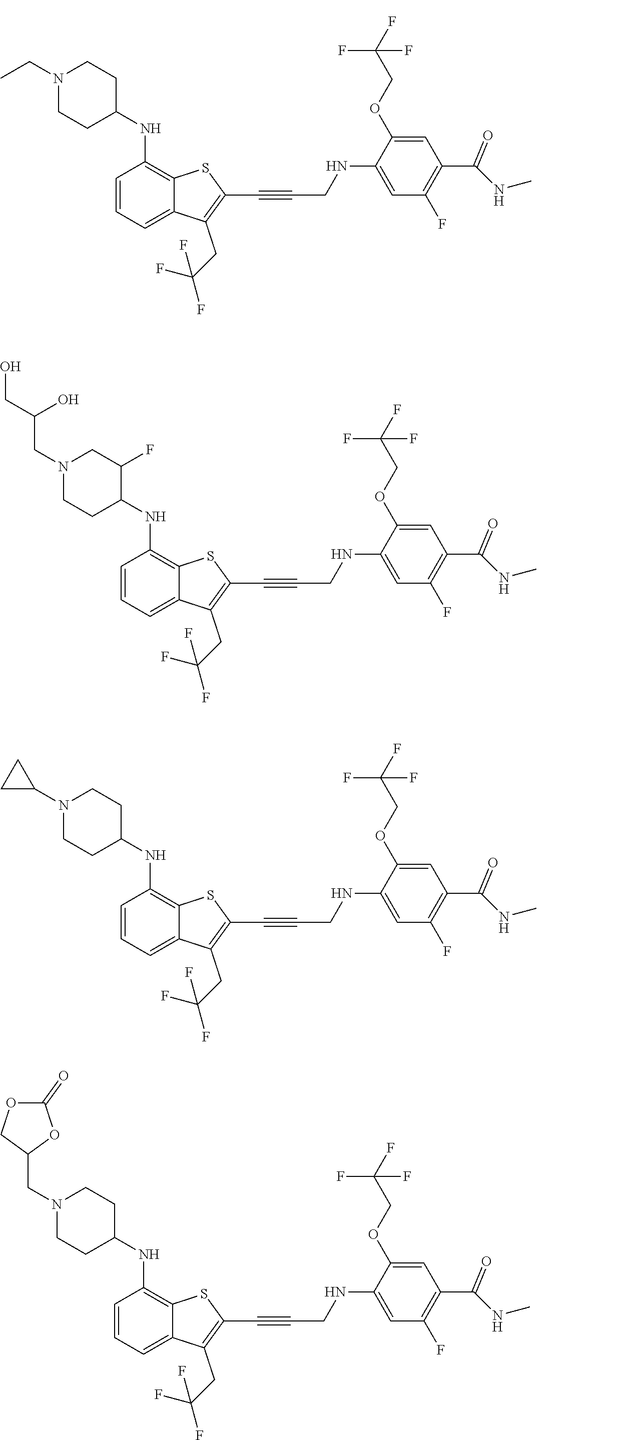 [SMILES: CCN1CCC(Nc2cccc3c(CC(F)(F)F)c(C#CCNc4cc(F)c(C(=O)NC)cc4OCC(F)(F)F)sc23)CC1 ; CNC(=O)c1cc(OCC(F)(F)F)c(NCC#Cc2sc3c(NC4CCN(CC(O)CO)CC4F)cccc3c2CC(F)(F)F)cc1F ; CNC(=O)c1cc(OCC(F)(F)F)c(NCC#Cc2sc3c(NC4CCN(C5CC5)CC4)cccc3c2CC(F)(F)F)cc1F ; CNC(=O)c1cc(OCC(F)(F)F)c(NCC#Cc2sc3c(NC4CCN(CC5COC(=O)O5)CC4)cccc3c2CC(F)(F)F)cc1F]

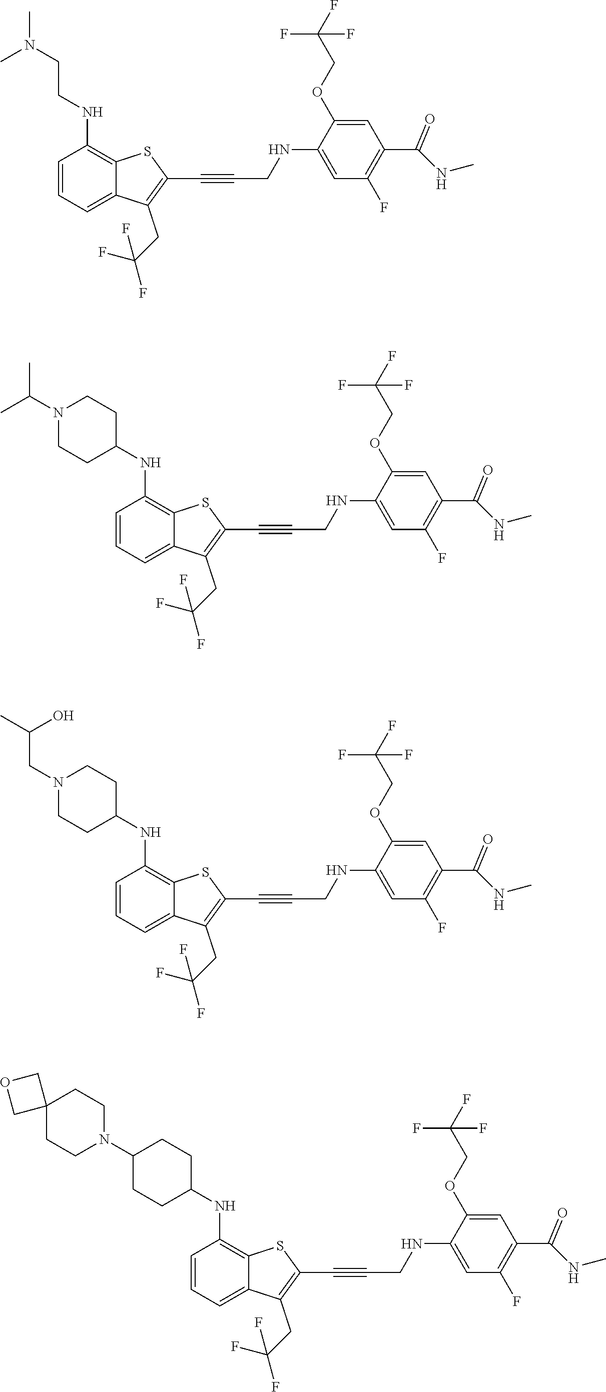

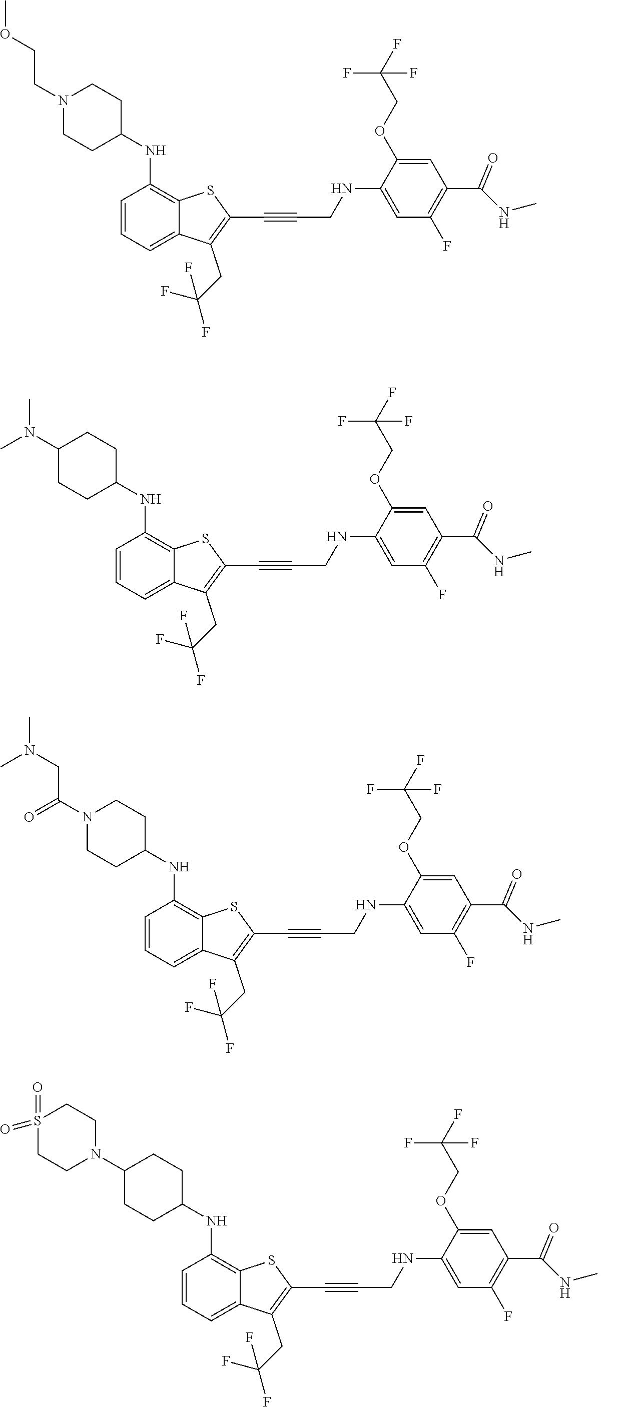

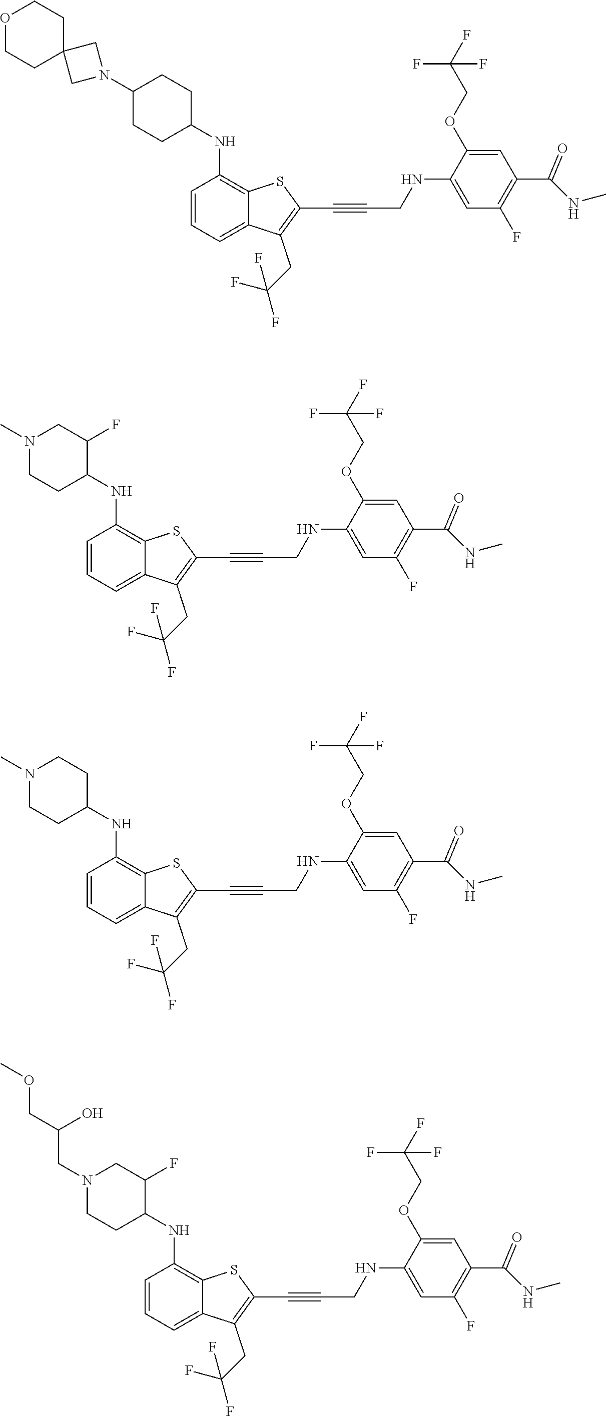

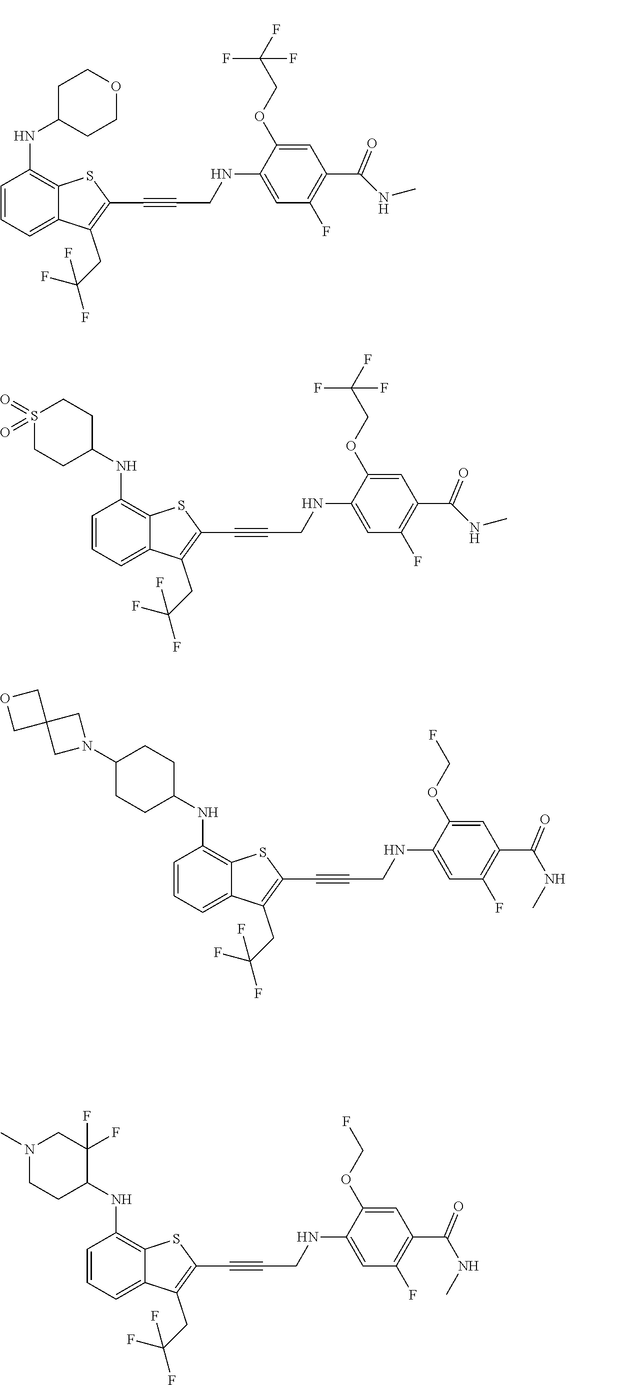

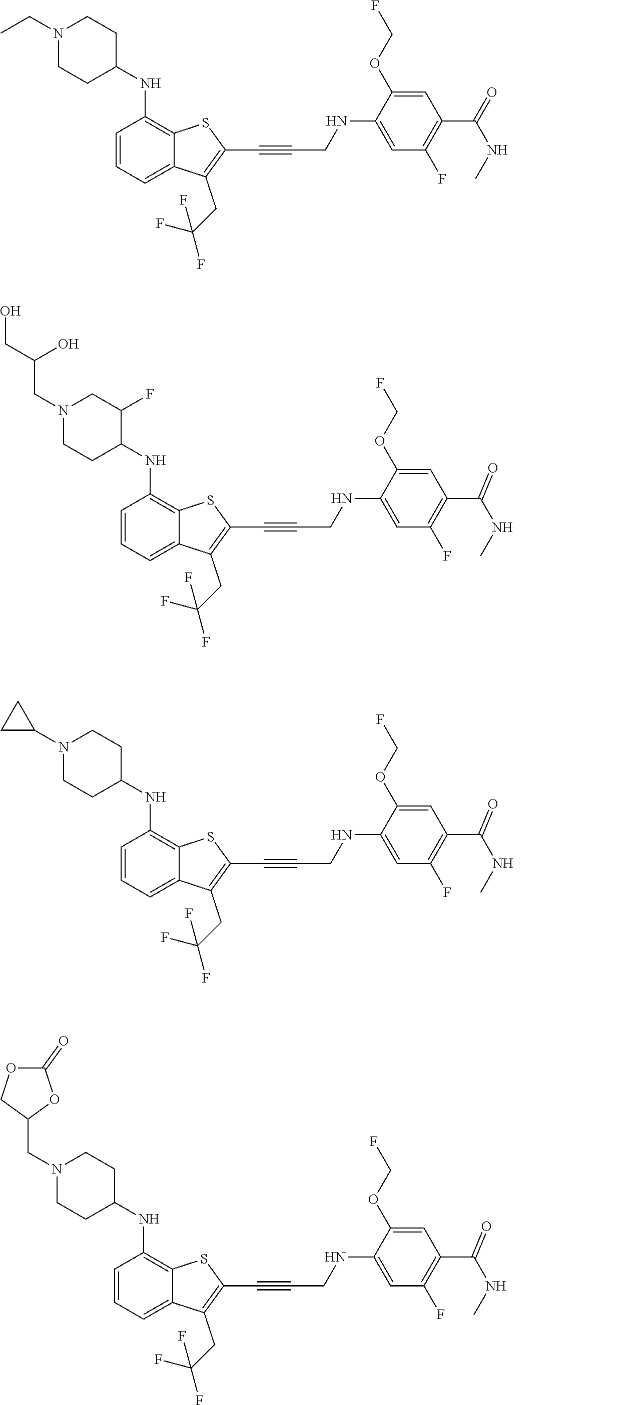 [SMILES: CCN1CCC(Nc2cccc3c(CC(F)(F)F)c(C#CCNc4cc(F)c(C(=O)NC)cc4OCF)sc23)CC1 ; CNC(=O)c1cc(OCF)c(NCC#Cc2sc3c(NC4CCN(CC(O)CO)CC4F)cccc3c2CC(F)(F)F)cc1F ; CNC(=O)c1cc(OCF)c(NCC#Cc2sc3c(NC4CCN(C5CC5)CC4)cccc3c2CC(F)(F)F)cc1F ; CNC(=O)c1cc(OCF)c(NCC#Cc2sc3c(NC4CCN(CC5COC(=O)O5)CC4)cccc3c2CC(F)(F)F)cc1F]

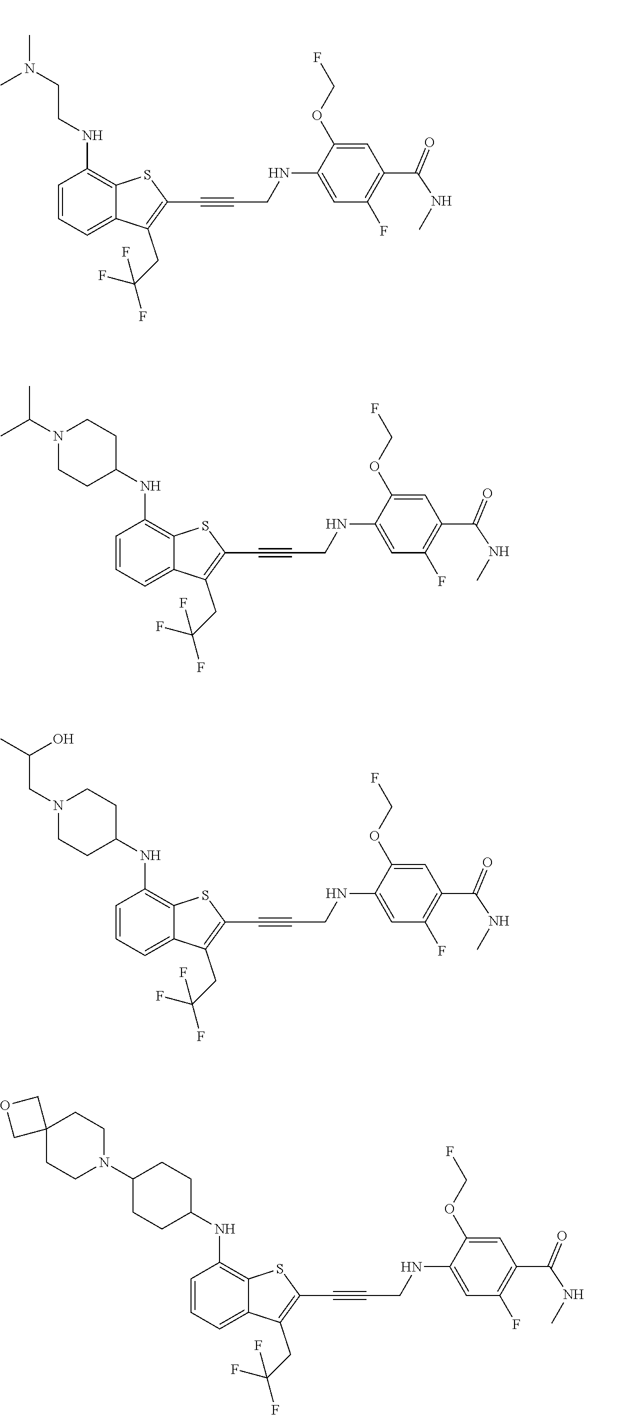 [SMILES: CNC(=O)c1cc(OCF)c(NCC#Cc2sc3c(NCCN(C)C)cccc3c2CC(F)(F)F)cc1F ; CNC(=O)c1cc(OCF)c(NCC#Cc2sc3c(NC4CCN(C(C)C)CC4)cccc3c2CC(F)(F)F)cc1F ; CNC(=O)c1cc(OCF)c(NCC#Cc2sc3c(NC4CCN(CC(C)O)CC4)cccc3c2CC(F)(F)F)cc1F ; CNC(=O)c1cc(OCF)c(NCC#Cc2sc3c(NC4CCC(N5CCC6(CC5)COC6)CC4)cccc3c2CC(F)(F)F)cc1F]

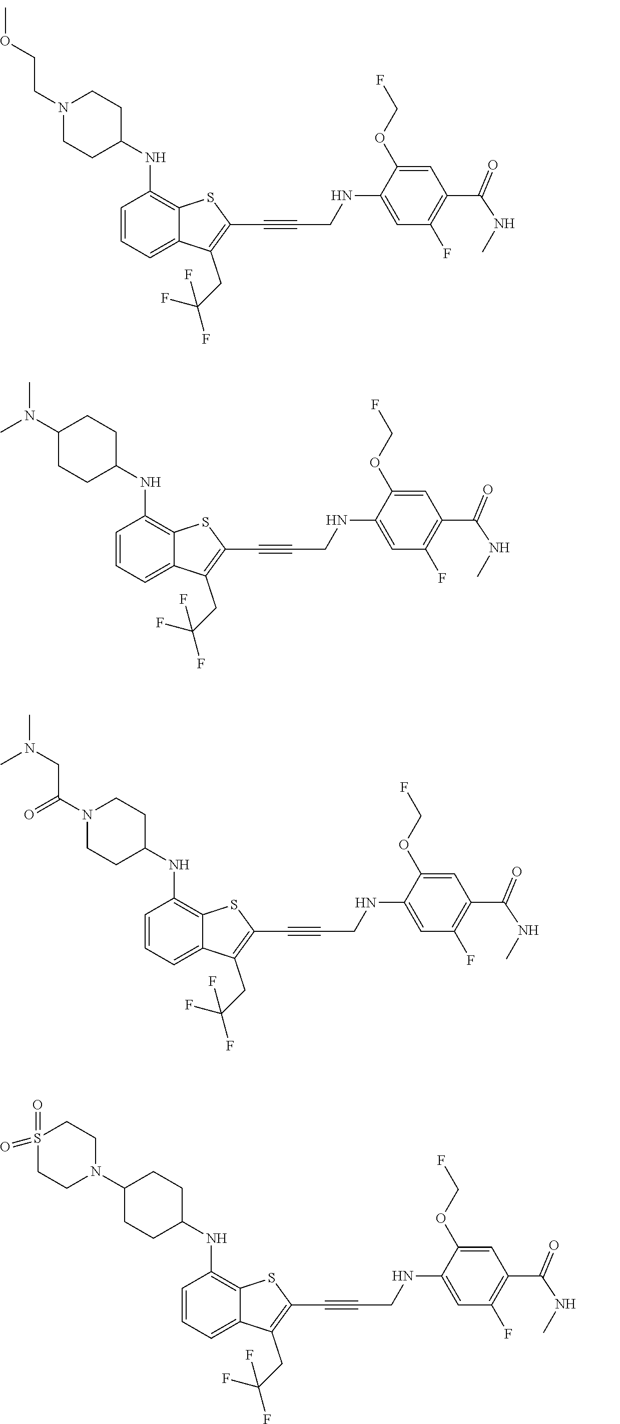 [SMILES: CNC(=O)c1cc(OCF)c(NCC#Cc2sc3c(NC4CCN(CCOC)CC4)cccc3c2CC(F)(F)F)cc1F ; CNC(=O)c1cc(OCF)c(NCC#Cc2sc3c(NC4CCC(N(C)C)CC4)cccc3c2CC(F)(F)F)cc1F ; CNC(=O)c1cc(OCF)c(NCC#Cc2sc3c(NC4CCN(C(=O)CN(C)C)CC4)cccc3c2CC(F)(F)F)cc1F ; CNC(=O)c1cc(OCF)c(NCC#Cc2sc3c(NC4CCC(N5CCS(=O)(=O)CC5)CC4)cccc3c2CC(F)(F)F)cc1F]

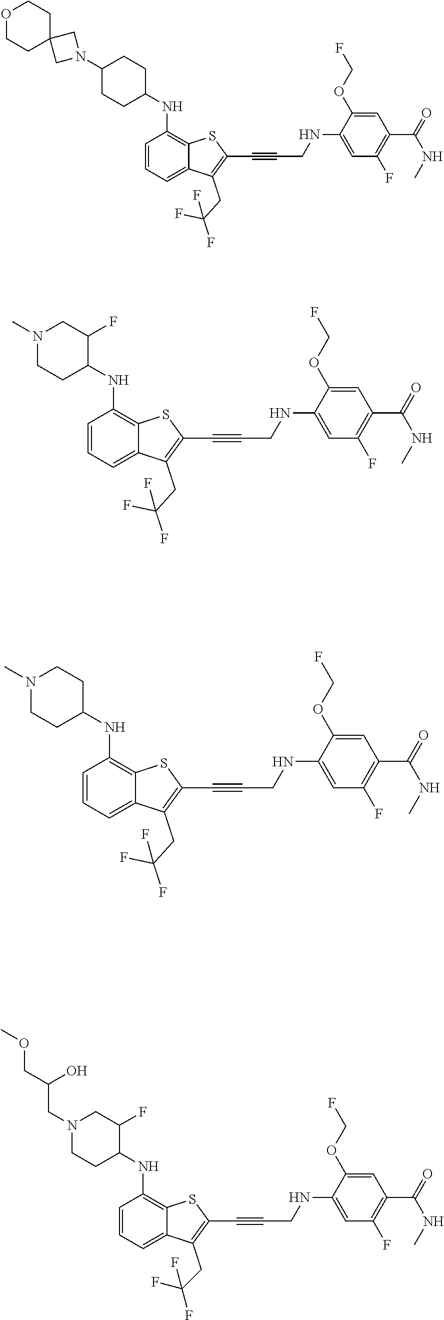 [SMILES: CNC(=O)c1cc(OCF)c(NCC#Cc2sc3c(NC4CCC(N5CC6(CCOCC6)C5)CC4)cccc3c2CC(F)(F)F)cc1F ; CNC(=O)c1cc(OCF)c(NCC#Cc2sc3c(NC4CCN(C)CC4F)cccc3c2CC(F)(F)F)cc1F ; CNC(=O)c1cc(OCF)c(NCC#Cc2sc3c(NC4CCN(C)CC4)cccc3c2CC(F)(F)F)cc1F ; CNC(=O)c1cc(OCF)c(NCC#Cc2sc3c(NC4CCN(CC(O)COC)CC4F)cccc3c2CC(F)(F)F)cc1F]

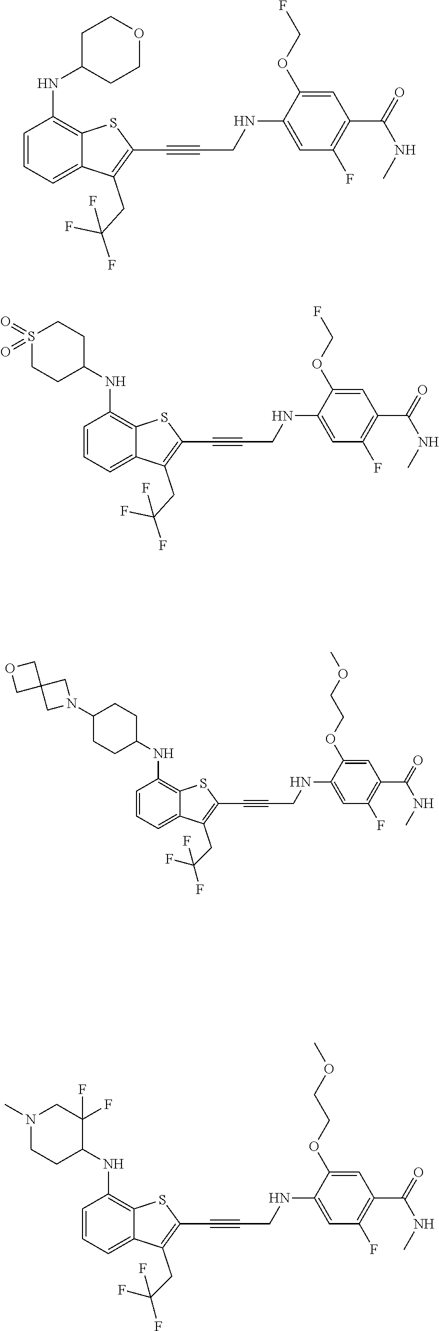 [SMILES: CNC(=O)c1cc(OCF)c(NCC#Cc2sc3c(NC4CCOCC4)cccc3c2CC(F)(F)F)cc1F ; CNC(=O)c1cc(OCF)c(NCC#Cc2sc3c(NC4CCS(=O)(=O)CC4)cccc3c2CC(F)(F)F)cc1F ; CNC(=O)c1cc(OCCOC)c(NCC#Cc2sc3c(NC4CCC(N5CC6(COC6)C5)CC4)cccc3c2CC(F)(F)F)cc1F ; CNC(=O)c1cc(OCCOC)c(NCC#Cc2sc3c(NC4CCN(C)CC4(F)F)cccc3c2CC(F)(F)F)cc1F]

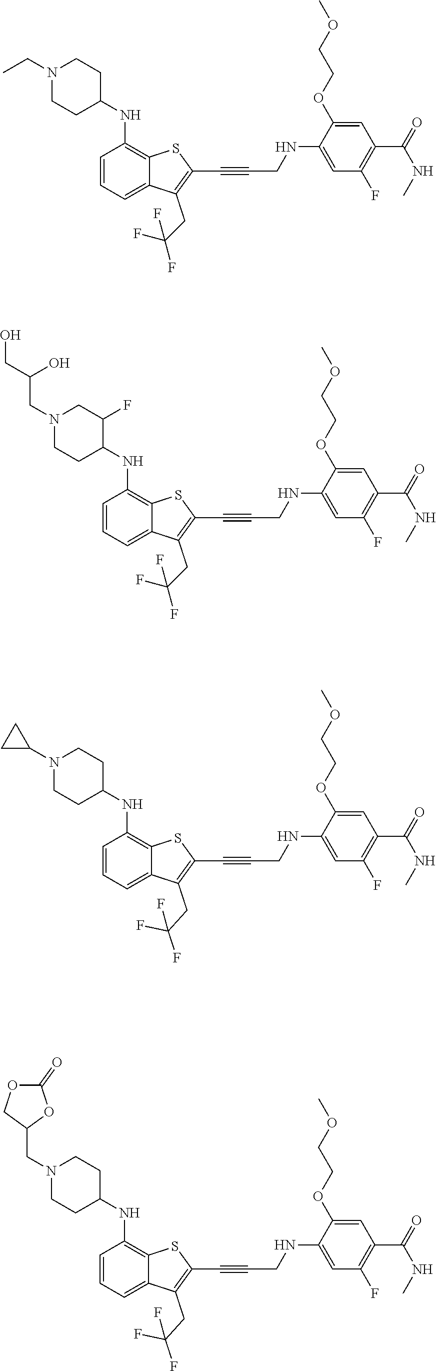 [SMILES: CCN1CCC(Nc2cccc3c(CC(F)(F)F)c(C#CCNc4cc(F)c(C(=O)NC)cc4OCCOC)sc23)CC1 ; CNC(=O)c1cc(OCCOC)c(NCC#Cc2sc3c(NC4CCN(CC(O)CO)CC4F)cccc3c2CC(F)(F)F)cc1F ; CNC(=O)c1cc(OCCOC)c(NCC#Cc2sc3c(NC4CCN(C5CC5)CC4)cccc3c2CC(F)(F)F)cc1F ; CNC(=O)c1cc(OCCOC)c(NCC#Cc2sc3c(NC4CCN(CC5COC(=O)O5)CC4)cccc3c2CC(F)(F)F)cc1F]

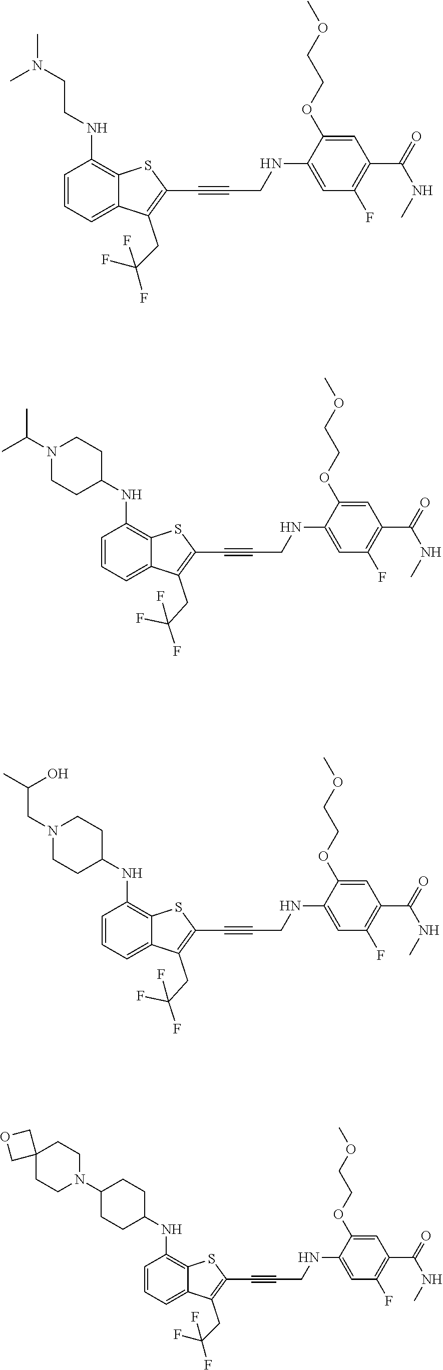 [SMILES: CNC(=O)c1cc(OCCOC)c(NCC#Cc2sc3c(NCCN(C)C)cccc3c2CC(F)(F)F)cc1F ; CNC(=O)c1cc(OCCOC)c(NCC#Cc2sc3c(NC4CCN(C(C)C)CC4)cccc3c2CC(F)(F)F)cc1F ; CNC(=O)c1cc(OCCOC)c(NCC#Cc2sc3c(NC4CCN(CC(C)O)CC4)cccc3c2CC(F)(F)F)cc1F ; CNC(=O)c1cc(OCCOC)c(NCC#Cc2sc3c(NC4CCC(N5CCC6(CC5)COC6)CC4)cccc3c2CC(F)(F)F)cc1F]

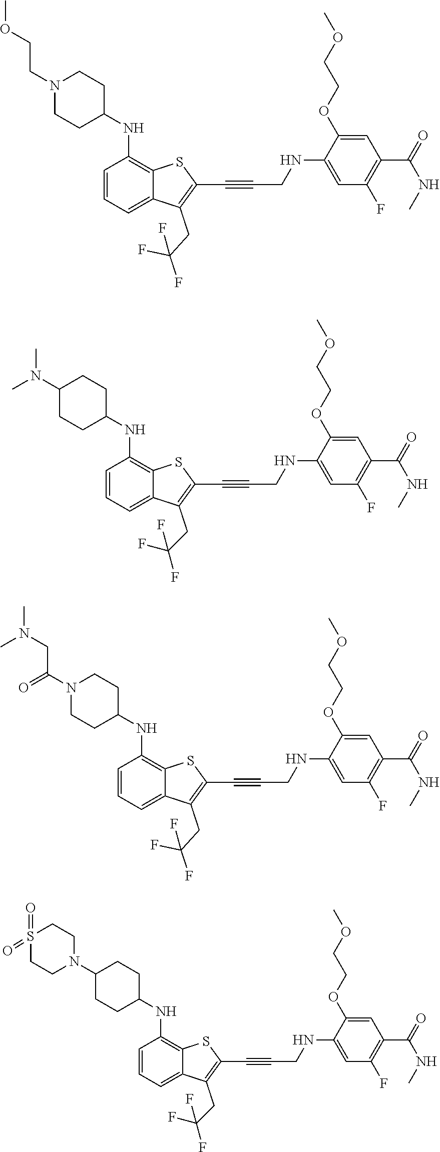 [SMILES: CNC(=O)c1cc(OCCOC)c(NCC#Cc2sc3c(NC4CCN(CCOC)CC4)cccc3c2CC(F)(F)F)cc1F ; CNC(=O)c1cc(OCCOC)c(NCC#Cc2sc3c(NC4CCC(N(C)C)CC4)cccc3c2CC(F)(F)F)cc1F ; CNC(=O)c1cc(OCCOC)c(NCC#Cc2sc3c(NC4CCN(C(=O)CN(C)C)CC4)cccc3c2CC(F)(F)F)cc1F ; CNC(=O)c1cc(OCCOC)c(NCC#Cc2sc3c(NC4CCC(N5CCS(=O)(=O)CC5)CC4)cccc3c2CC(F)(F)F)cc1F]

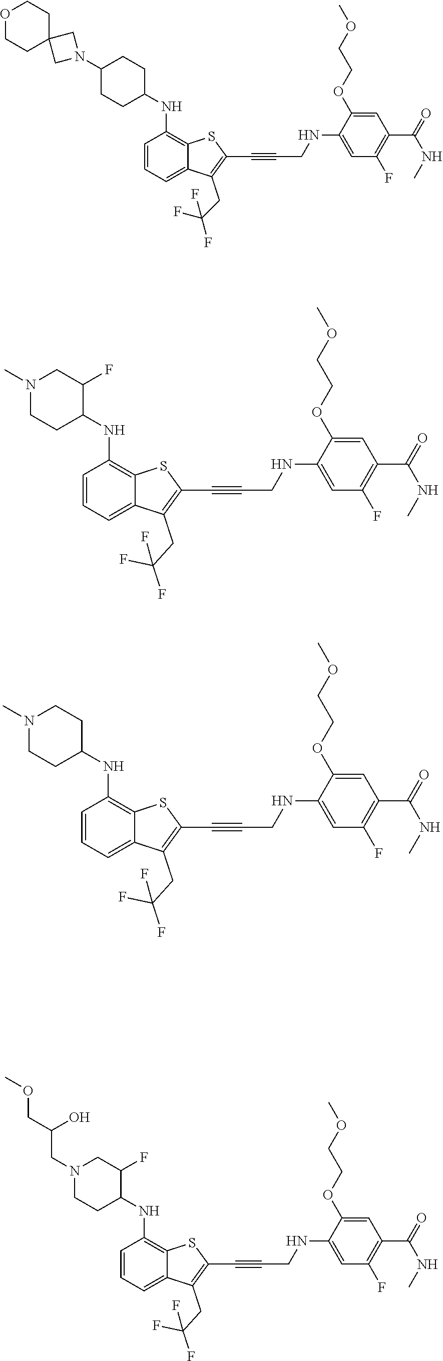 [SMILES: CNC(=O)c1cc(OCCOC)c(NCC#Cc2sc3c(NC4CCC(N5CC6(CCOCC6)C5)CC4)cccc3c2CC(F)(F)F)cc1F ; CNC(=O)c1cc(OCCOC)c(NCC#Cc2sc3c(NC4CCN(C)CC4F)cccc3c2CC(F)(F)F)cc1F ; CNC(=O)c1cc(OCCOC)c(NCC#Cc2sc3c(NC4CCN(C)CC4)cccc3c2CC(F)(F)F)cc1F ; CNC(=O)c1cc(OCCOC)c(NCC#Cc2sc3c(NC4CCN(CC(O)COC)CC4F)cccc3c2CC(F)(F)F)cc1F]

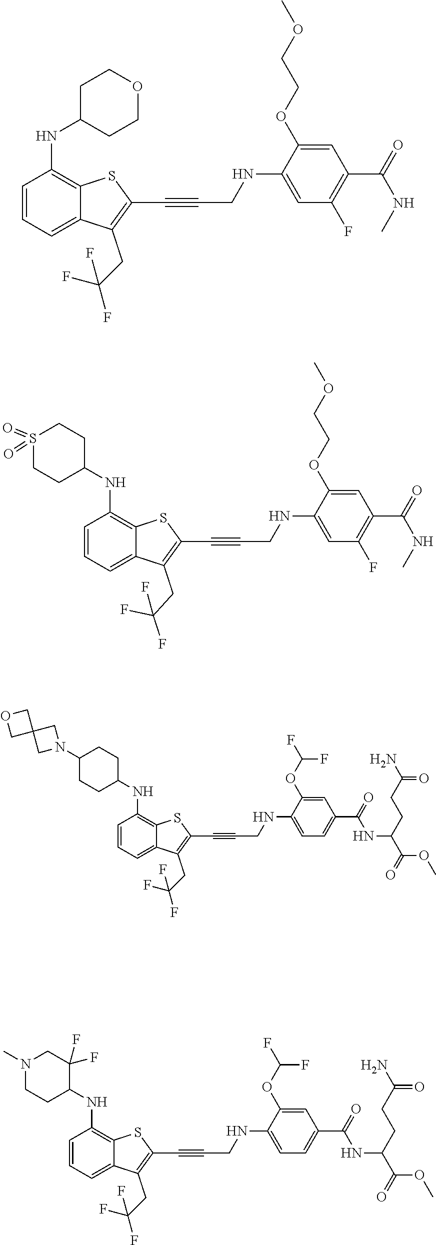

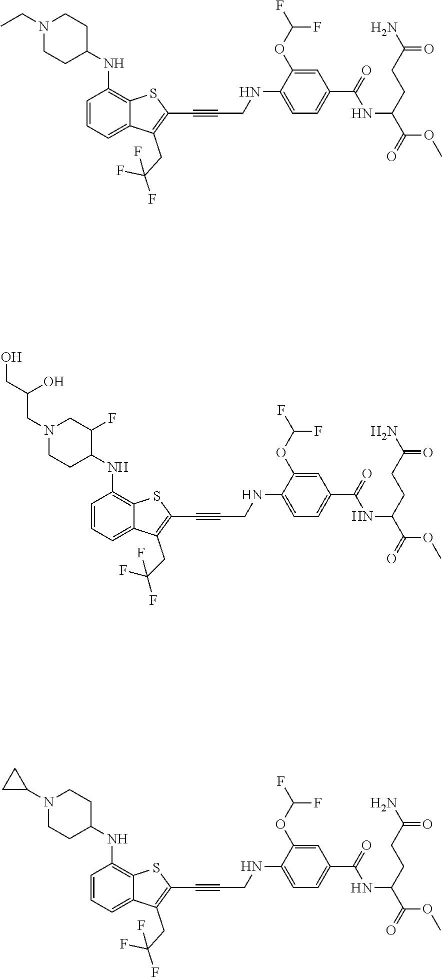 [SMILES: CCN1CCC(Nc2cccc3c(CC(F)(F)F)c(C#CCNc4ccc(C(=O)NC(CCC(N)=O)C(=O)OC)cc4OC(F)F)sc23)CC1 ; COC(=O)C(CCC(N)=O)NC(=O)c1ccc(NCC#Cc2sc3c(NC4CCN(CC(O)CO)CC4F)cccc3c2CC(F)(F)F)c(OC(F)F)c1 ; COC(=O)C(CCC(N)=O)NC(=O)c1ccc(NCC#Cc2sc3c(NC4CCN(C5CC5)CC4)cccc3c2CC(F)(F)F)c(OC(F)F)c1]

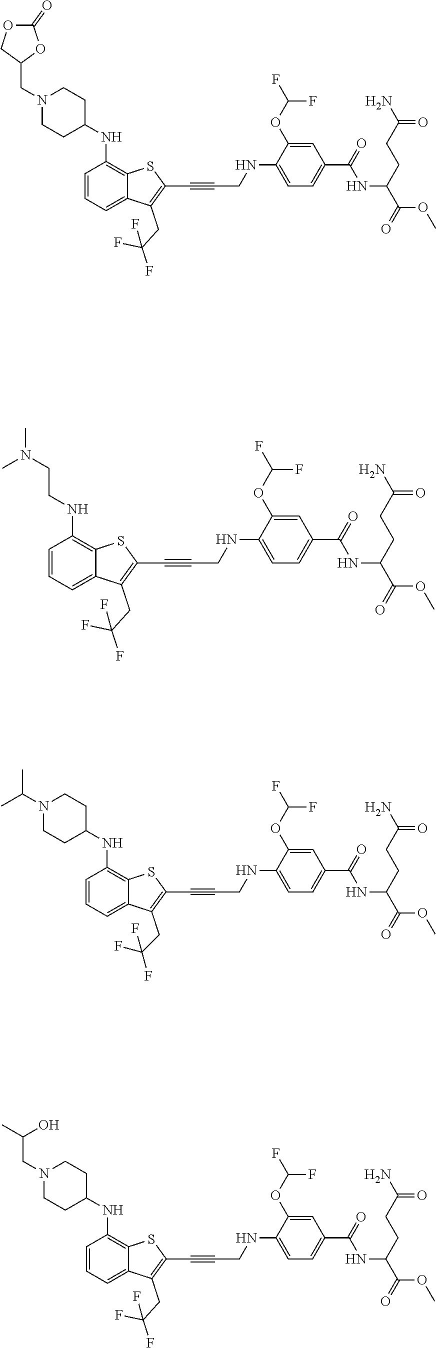

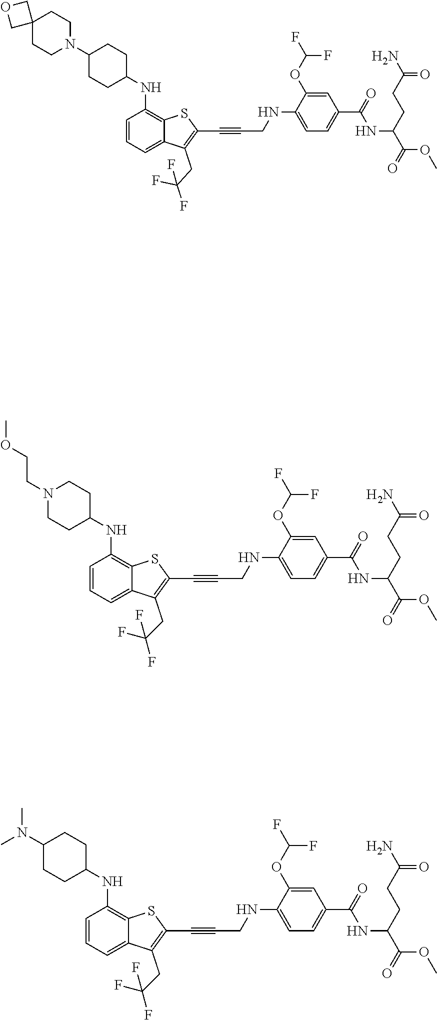

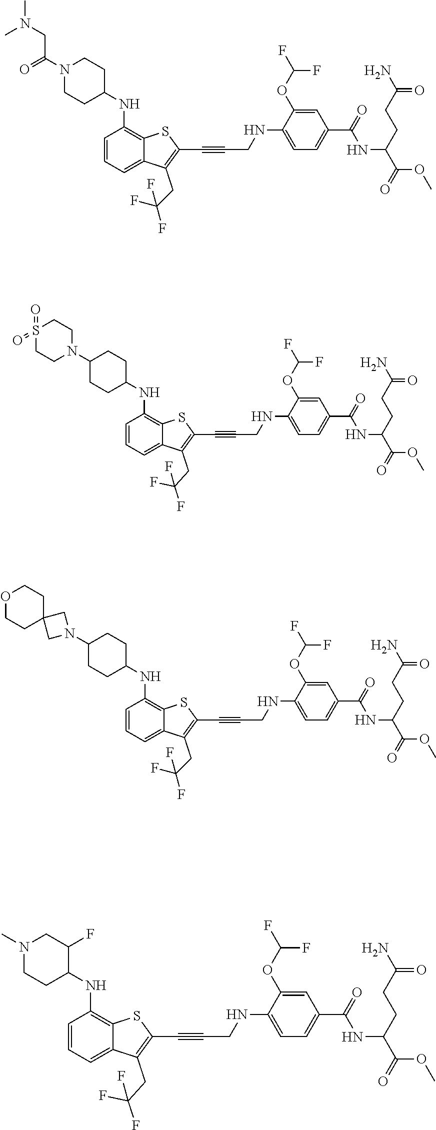

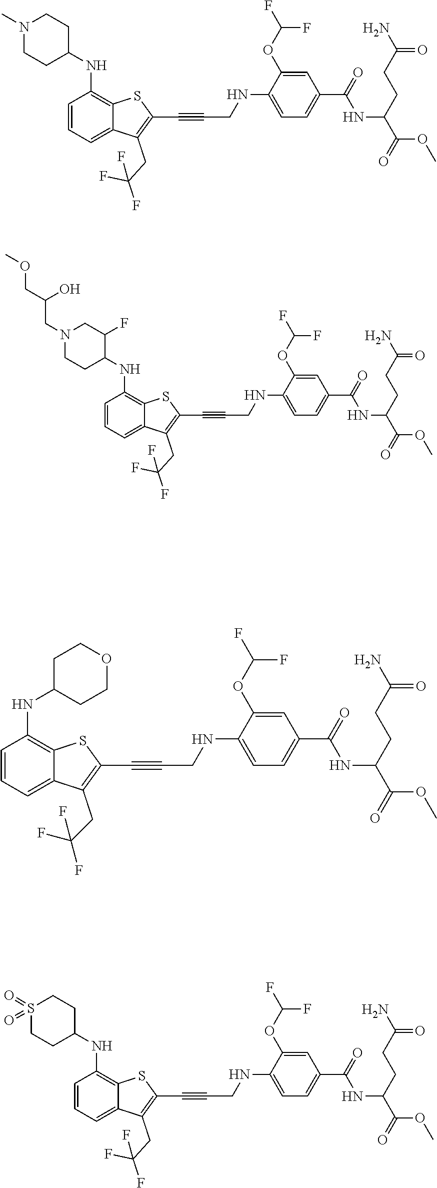

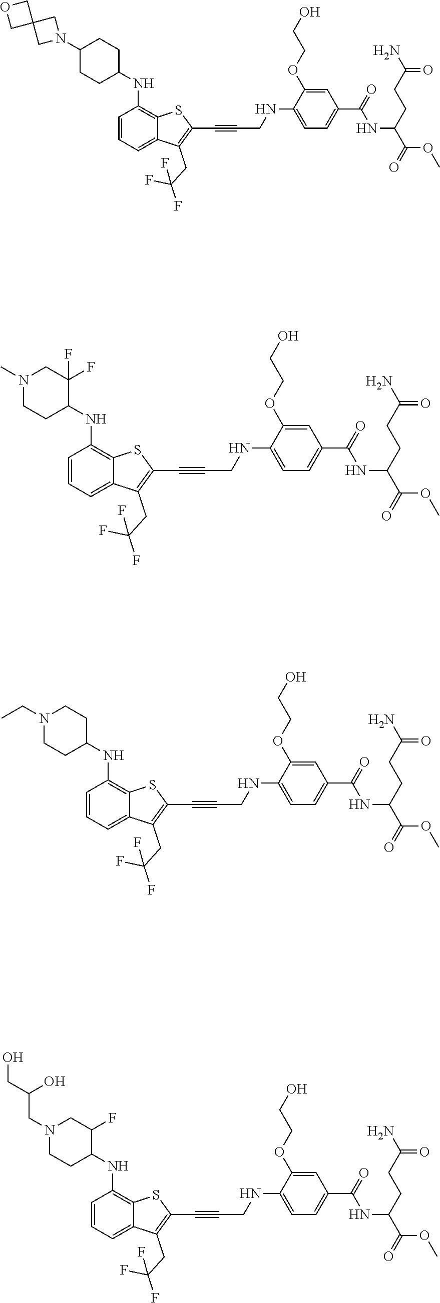

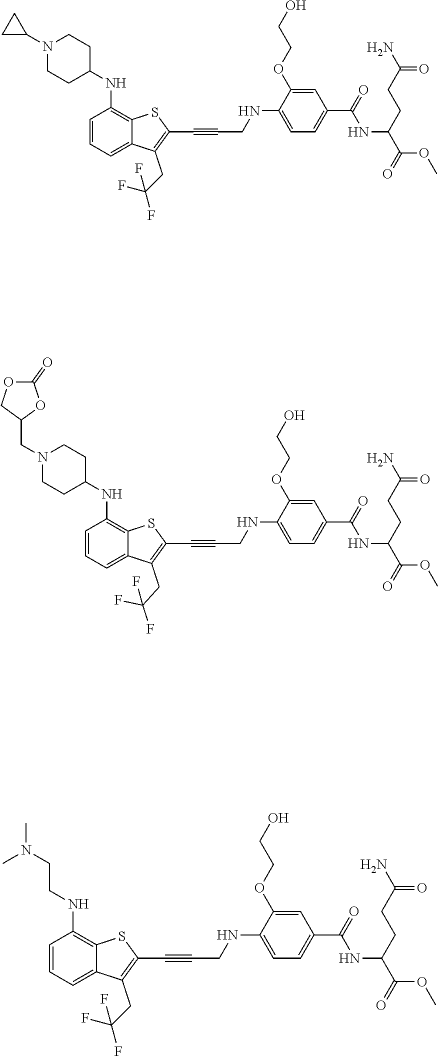

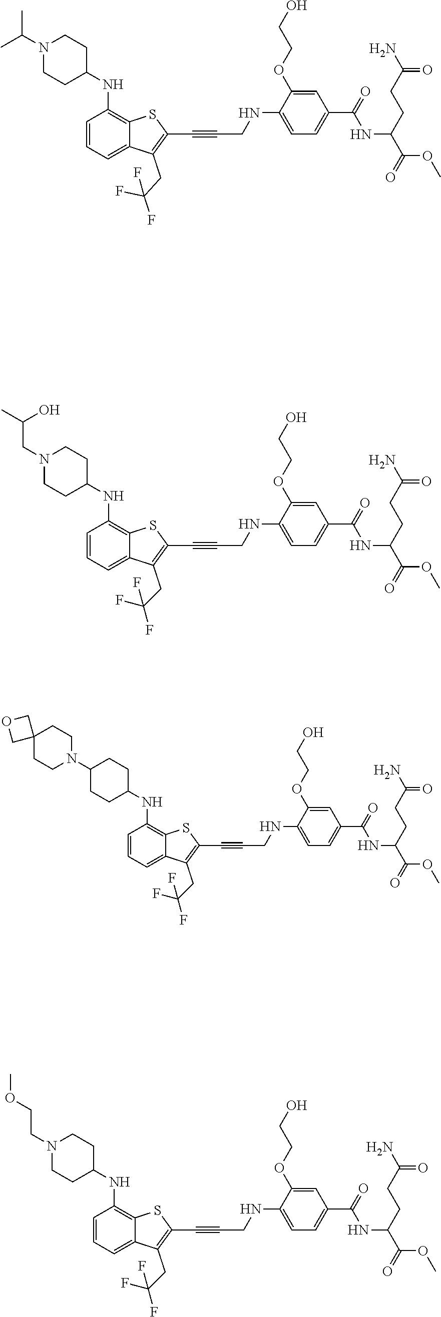

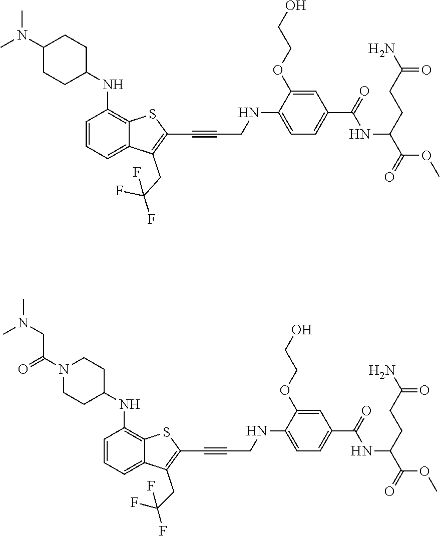

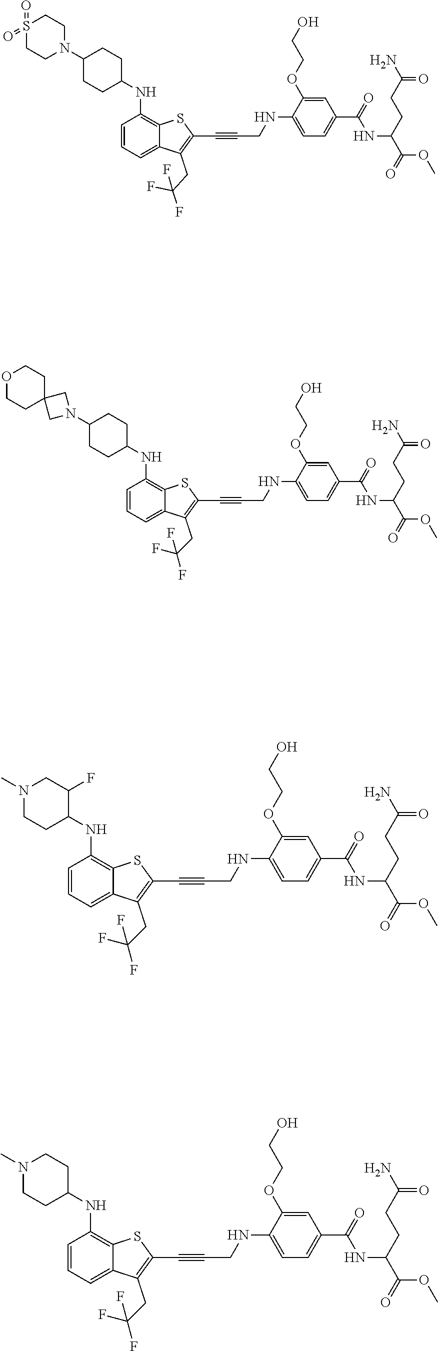

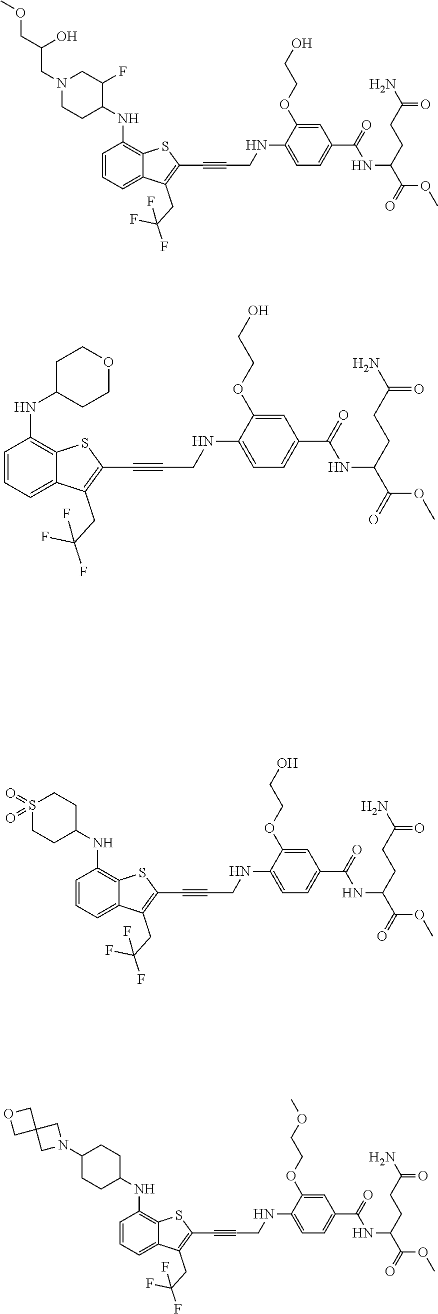

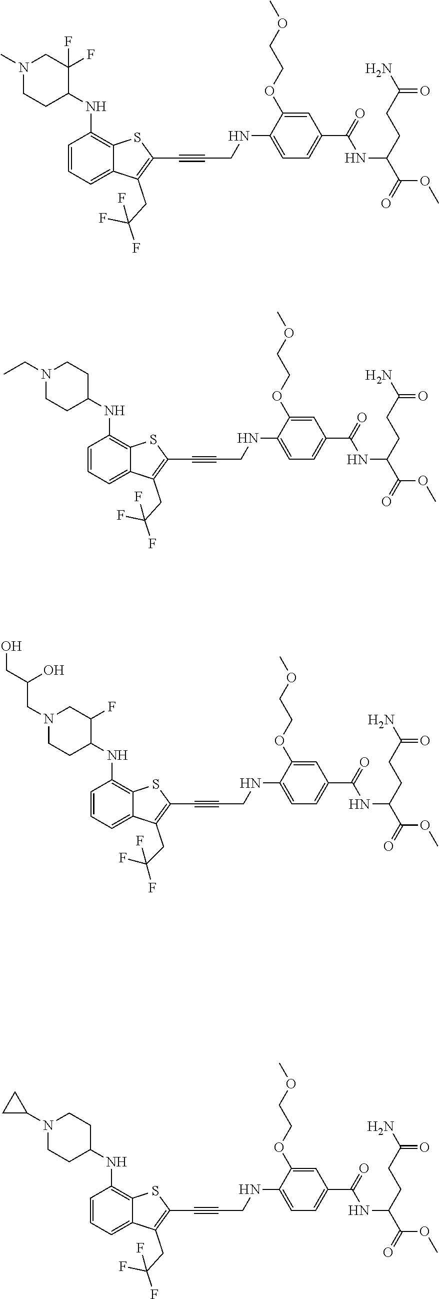

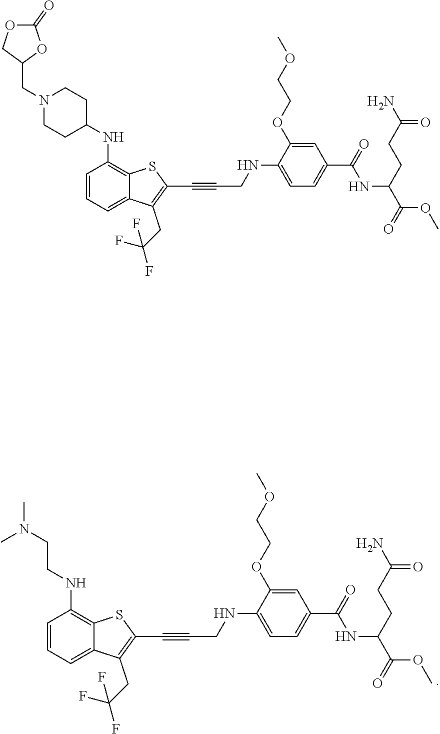

[57]. The compound according to any one of [1] to [53], wherein the compound of formula (I) is selected from:

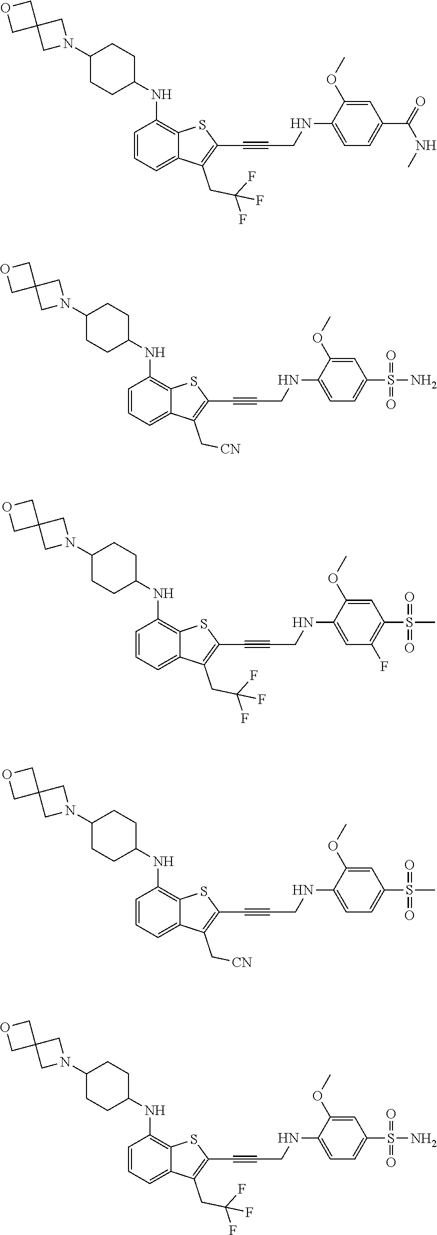 [SMILES: CNC(=O)c1ccc(NCC#Cc2sc3c(NC4CCC(N5CC6(COC6)C5)CC4)cccc3c2CC(F)(F)F)c(OC)c1 ; COc1cc(S(N)(=O)=O)ccc1NCC#Cc1sc2c(NC3CCC(N4CC5(COC5)C4)CC3)cccc2c1CC#N ; COc1cc(S(C)(=O)=O)c(F)cc1NCC#Cc1sc2c(NC3CCC(N4CC5(COC5)C4)CC3)cccc2c1CC(F)(F)F ; COc1cc(S(C)(=O)=O)ccc1NCC#Cc1sc2c(NC3CCC(N4CC5(COC5)C4)CC3)cccc2c1CC#N ; COc1cc(S(N)(=O)=O)ccc1NCC#Cc1sc2c(NC3CCC(N4CC5(COC5)C4)CC3)cccc2c1CC(F)(F)F]

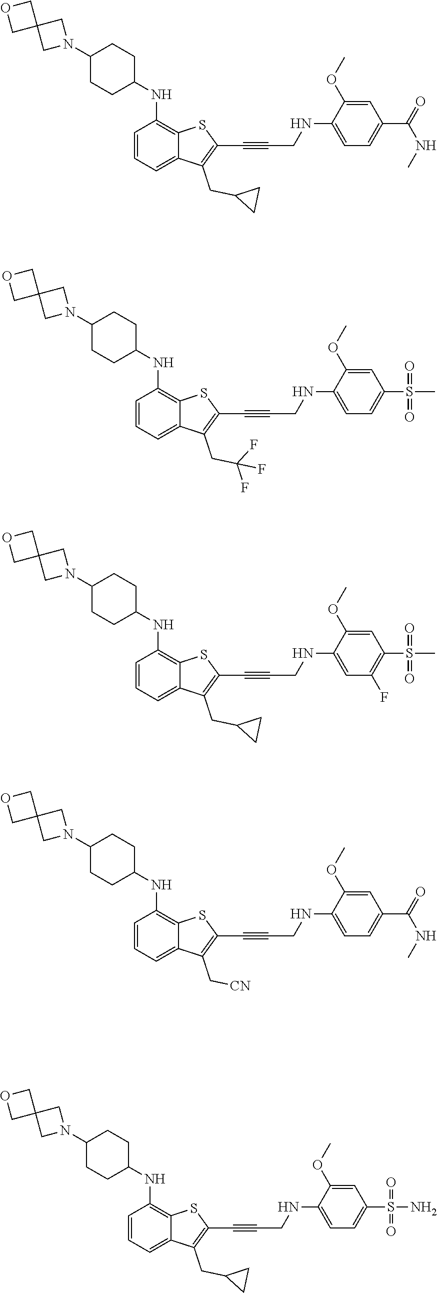 [SMILES: CNC(=O)c1ccc(NCC#Cc2sc3c(NC4CCC(N5CC6(COC6)C5)CC4)cccc3c2CC2CC2)c(OC)c1 ; COc1cc(S(C)(=O)=O)ccc1NCC#Cc1sc2c(NC3CCC(N4CC5(COC5)C4)CC3)cccc2c1CC(F)(F)F ; COc1cc(S(C)(=O)=O)c(F)cc1NCC#Cc1sc2c(NC3CCC(N4CC5(COC5)C4)CC3)cccc2c1CC1CC1 ; CNC(=O)c1ccc(NCC#Cc2sc3c(NC4CCC(N5CC6(COC6)C5)CC4)cccc3c2CC#N)c(OC)c1 ; COc1cc(S(N)(=O)=O)ccc1NCC#Cc1sc2c(NC3CCC(N4CC5(COC5)C4)CC3)cccc2c1CC1CC1]

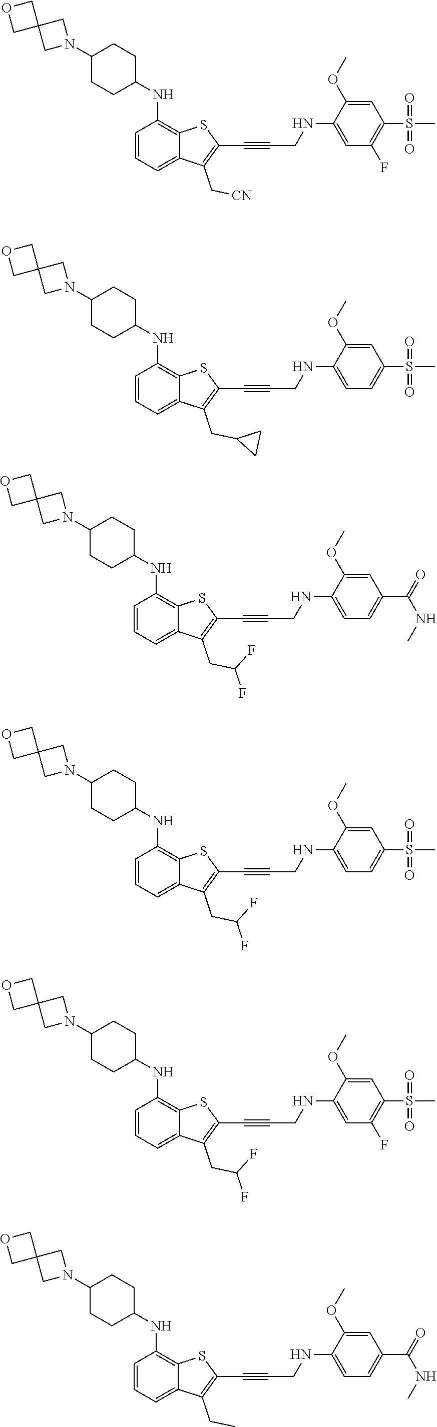 [SMILES: COc1cc(S(C)(=O)=O)c(F)cc1NCC#Cc1sc2c(NC3CCC(N4CC5(COC5)C4)CC3)cccc2c1CC#N ; COc1cc(S(C)(=O)=O)ccc1NCC#Cc1sc2c(NC3CCC(N4CC5(COC5)C4)CC3)cccc2c1CC1CC1 ; CNC(=O)c1ccc(NCC#Cc2sc3c(NC4CCC(N5CC6(COC6)C5)CC4)cccc3c2CC(F)F)c(OC)c1 ; COc1cc(S(C)(=O)=O)ccc1NCC#Cc1sc2c(NC3CCC(N4CC5(COC5)C4)CC3)cccc2c1CC(F)F ; COc1cc(S(C)(=O)=O)c(F)cc1NCC#Cc1sc2c(NC3CCC(N4CC5(COC5)C4)CC3)cccc2c1CC(F)F ; CCc1c(C#CCNc2ccc(C(=O)NC)cc2OC)sc2c(NC3CCC(N4CC5(COC5)C4)CC3)cccc12]

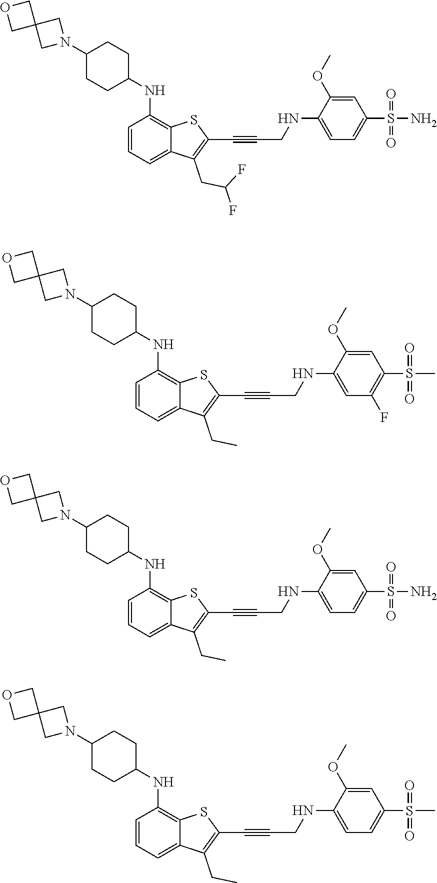 [SMILES: COc1cc(S(N)(=O)=O)ccc1NCC#Cc1sc2c(NC3CCC(N4CC5(COC5)C4)CC3)cccc2c1CC(F)F ; CCc1c(C#CCNc2cc(F)c(S(C)(=O)=O)cc2OC)sc2c(NC3CCC(N4CC5(COC5)C4)CC3)cccc12 ; CCc1c(C#CCNc2ccc(S(N)(=O)=O)cc2OC)sc2c(NC3CCC(N4CC5(COC5)C4)CC3)cccc12 ; CCc1c(C#CCNc2ccc(S(C)(=O)=O)cc2OC)sc2c(NC3CCC(N4CC5(COC5)C4)CC3)cccc12]

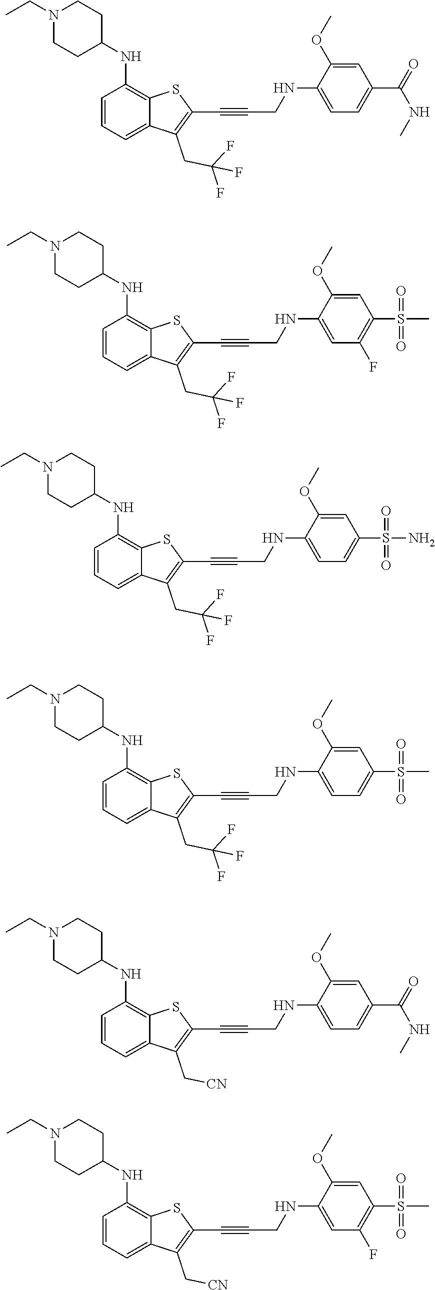

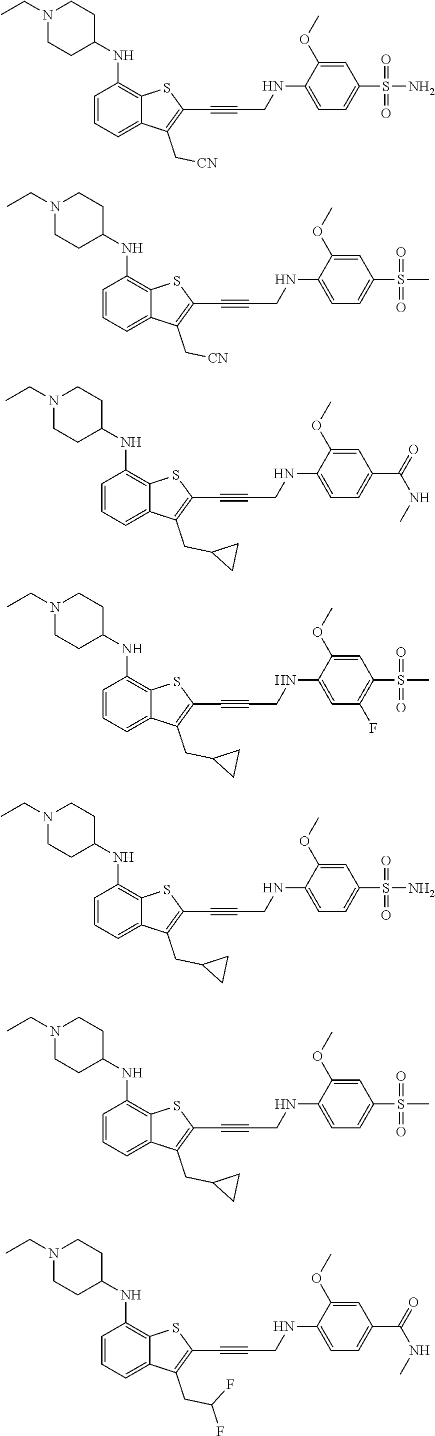

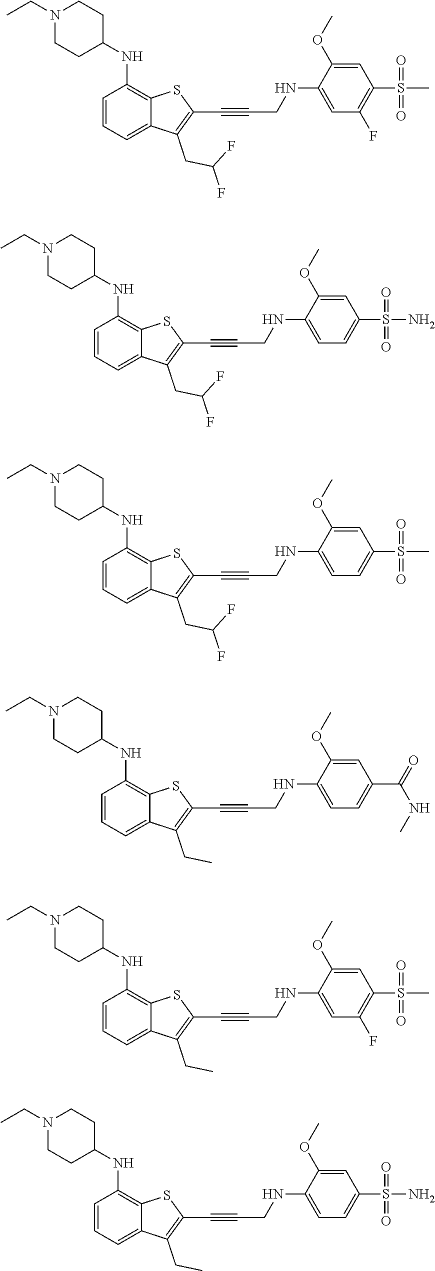

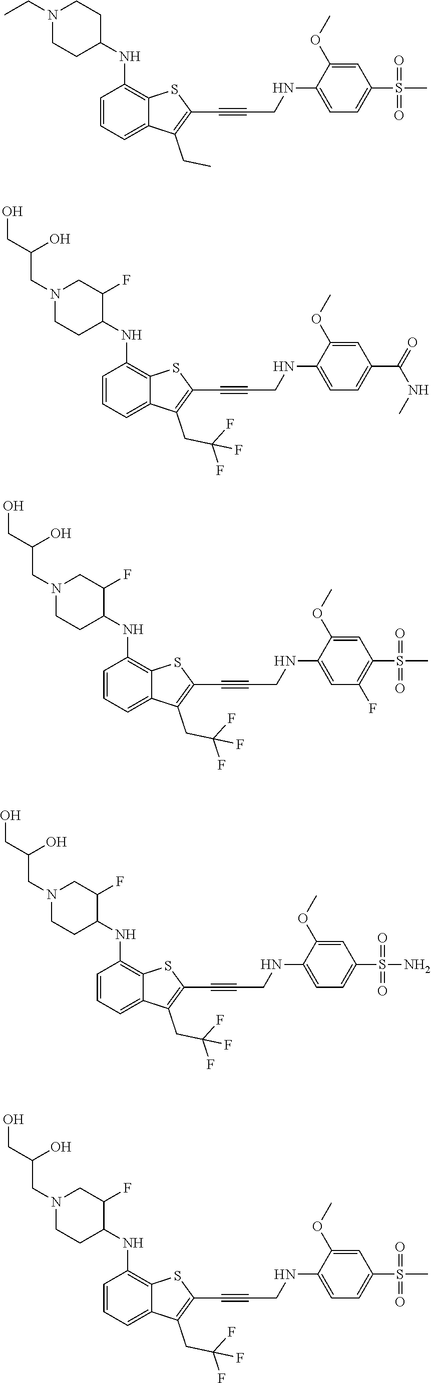 [SMILES: CCc1c(C#CCNc2ccc(S(C)(=O)=O)cc2OC)sc2c(NC3CCN(CC)CC3)cccc12 ; CNC(=O)c1ccc(NCC#Cc2sc3c(NC4CCN(CC(O)CO)CC4F)cccc3c2CC(F)(F)F)c(OC)c1 ; COc1cc(S(C)(=O)=O)c(F)cc1NCC#Cc1sc2c(NC3CCN(CC(O)CO)CC3F)cccc2c1CC(F)(F)F ; COc1cc(S(N)(=O)=O)ccc1NCC#Cc1sc2c(NC3CCN(CC(O)CO)CC3F)cccc2c1CC(F)(F)F ; COc1cc(S(C)(=O)=O)ccc1NCC#Cc1sc2c(NC3CCN(CC(O)CO)CC3F)cccc2c1CC(F)(F)F]

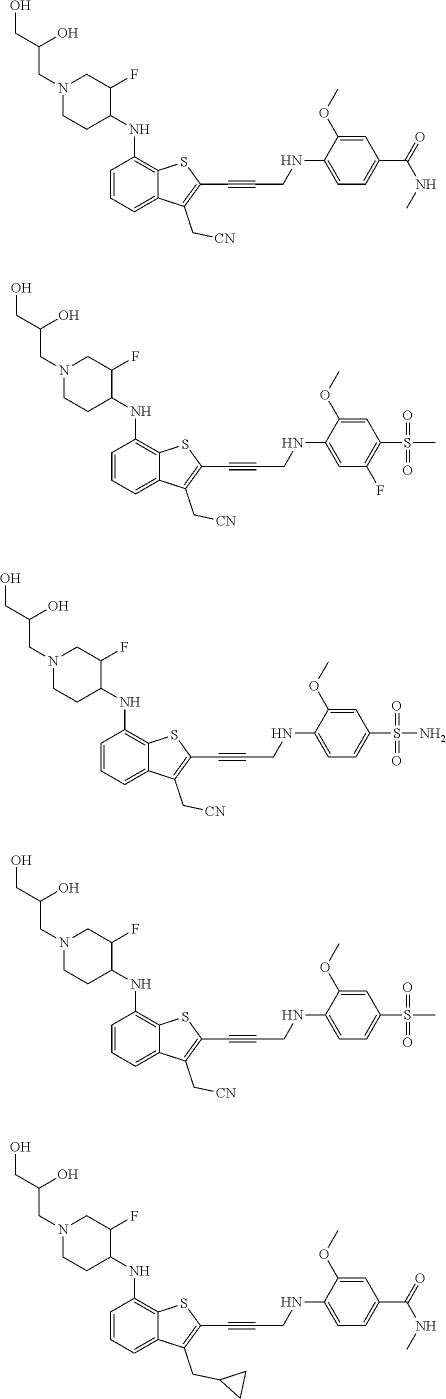 [SMILES: CNC(=O)c1ccc(NCC#Cc2sc3c(NC4CCN(CC(O)CO)CC4F)cccc3c2CC#N)c(OC)c1 ; COc1cc(S(C)(=O)=O)c(F)cc1NCC#Cc1sc2c(NC3CCN(CC(O)CO)CC3F)cccc2c1CC#N ; COc1cc(S(N)(=O)=O)ccc1NCC#Cc1sc2c(NC3CCN(CC(O)CO)CC3F)cccc2c1CC#N ; COc1cc(S(C)(=O)=O)ccc1NCC#Cc1sc2c(NC3CCN(CC(O)CO)CC3F)cccc2c1CC#N ; CNC(=O)c1ccc(NCC#Cc2sc3c(NC4CCN(CC(O)CO)CC4F)cccc3c2CC2CC2)c(OC)c1]

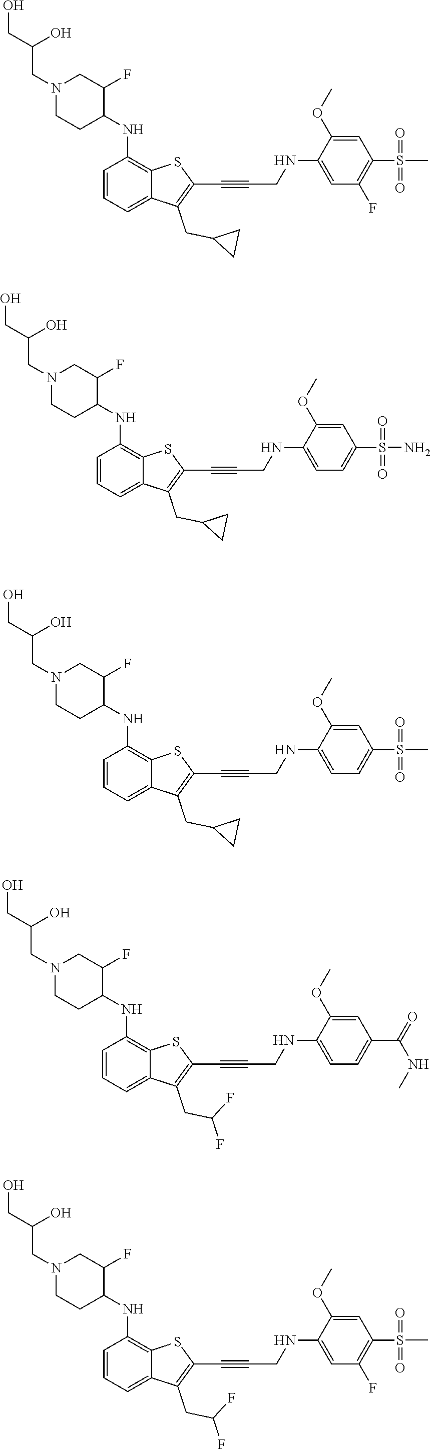 [SMILES: COc1cc(S(C)(=O)=O)c(F)cc1NCC#Cc1sc2c(NC3CCN(CC(O)CO)CC3F)cccc2c1CC1CC1 ; COc1cc(S(N)(=O)=O)ccc1NCC#Cc1sc2c(NC3CCN(CC(O)CO)CC3F)cccc2c1CC1CC1 ; COc1cc(S(C)(=O)=O)ccc1NCC#Cc1sc2c(NC3CCN(CC(O)CO)CC3F)cccc2c1CC1CC1 ; CNC(=O)c1ccc(NCC#Cc2sc3c(NC4CCN(CC(O)CO)CC4F)cccc3c2CC(F)F)c(OC)c1 ; COc1cc(S(C)(=O)=O)c(F)cc1NCC#Cc1sc2c(NC3CCN(CC(O)CO)CC3F)cccc2c1CC(F)F]

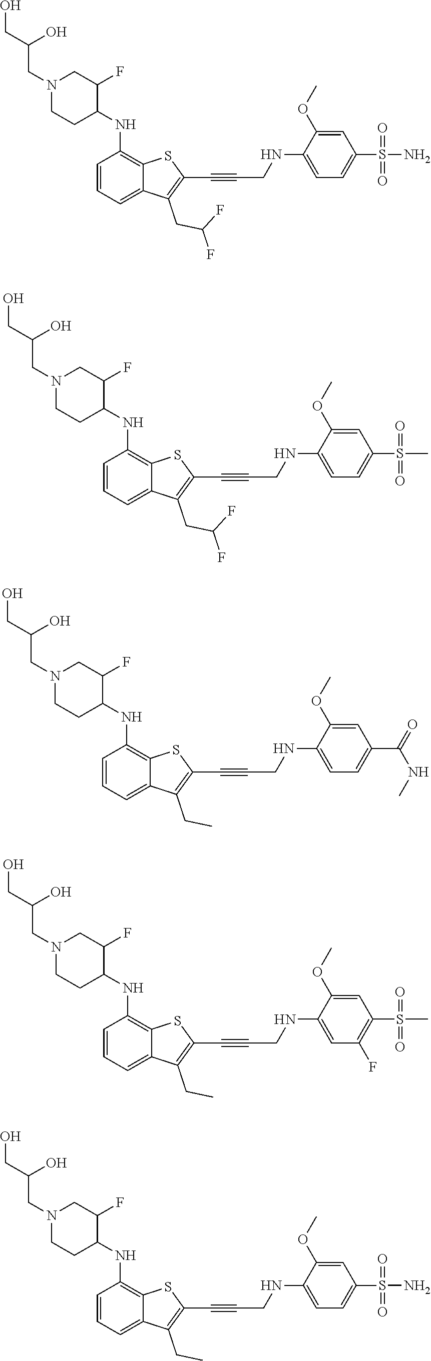 [SMILES: COc1cc(S(N)(=O)=O)ccc1NCC#Cc1sc2c(NC3CCN(CC(O)CO)CC3F)cccc2c1CC(F)F ; COc1cc(S(C)(=O)=O)ccc1NCC#Cc1sc2c(NC3CCN(CC(O)CO)CC3F)cccc2c1CC(F)F ; CCc1c(C#CCNc2ccc(C(=O)NC)cc2OC)sc2c(NC3CCN(CC(O)CO)CC3F)cccc12 ; CCc1c(C#CCNc2cc(F)c(S(C)(=O)=O)cc2OC)sc2c(NC3CCN(CC(O)CO)CC3F)cccc12 ; CCc1c(C#CCNc2ccc(S(N)(=O)=O)cc2OC)sc2c(NC3CCN(CC(O)CO)CC3F)cccc12]

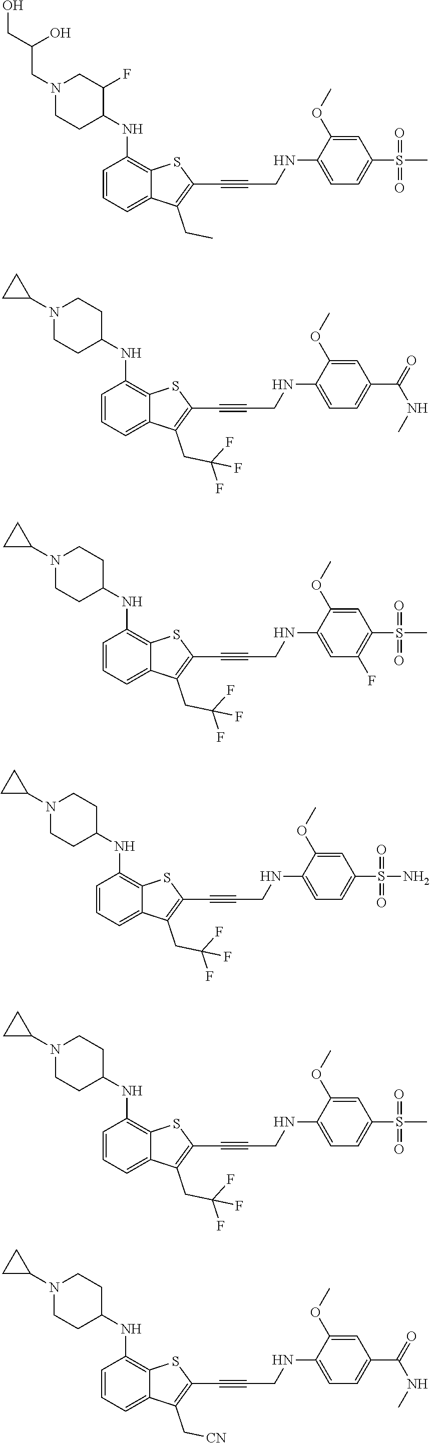 [SMILES: CCc1c(C#CCNc2ccc(S(C)(=O)=O)cc2OC)sc2c(NC3CCN(CC(O)CO)CC3F)cccc12 ; CNC(=O)c1ccc(NCC#Cc2sc3c(NC4CCN(C5CC5)CC4)cccc3c2CC(F)(F)F)c(OC)c1 ; COc1cc(S(C)(=O)=O)c(F)cc1NCC#Cc1sc2c(NC3CCN(C4CC4)CC3)cccc2c1CC(F)(F)F ; COc1cc(S(N)(=O)=O)ccc1NCC#Cc1sc2c(NC3CCN(C4CC4)CC3)cccc2c1CC(F)(F)F ; COc1cc(S(C)(=O)=O)ccc1NCC#Cc1sc2c(NC3CCN(C4CC4)CC3)cccc2c1CC(F)(F)F ; CNC(=O)c1ccc(NCC#Cc2sc3c(NC4CCN(C5CC5)CC4)cccc3c2CC#N)c(OC)c1]

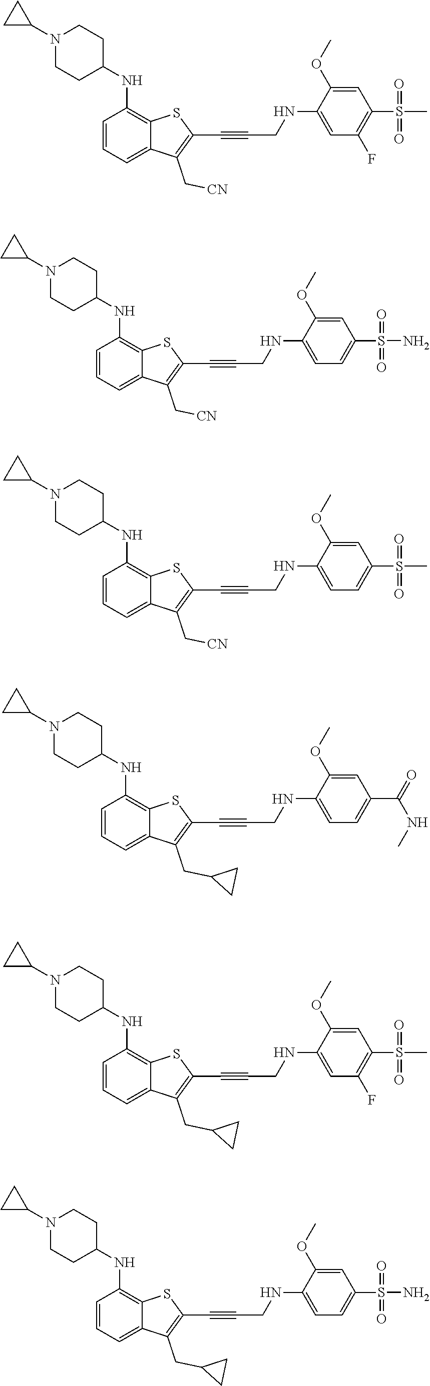 [SMILES: COc1cc(S(C)(=O)=O)c(F)cc1NCC#Cc1sc2c(NC3CCN(C4CC4)CC3)cccc2c1CC#N ; COc1cc(S(N)(=O)=O)ccc1NCC#Cc1sc2c(NC3CCN(C4CC4)CC3)cccc2c1CC#N ; COc1cc(S(C)(=O)=O)ccc1NCC#Cc1sc2c(NC3CCN(C4CC4)CC3)cccc2c1CC#N ; CNC(=O)c1ccc(NCC#Cc2sc3c(NC4CCN(C5CC5)CC4)cccc3c2CC2CC2)c(OC)c1 ; COc1cc(S(C)(=O)=O)c(F)cc1NCC#Cc1sc2c(NC3CCN(C4CC4)CC3)cccc2c1CC1CC1 ; COc1cc(S(N)(=O)=O)ccc1NCC#Cc1sc2c(NC3CCN(C4CC4)CC3)cccc2c1CC1CC1]

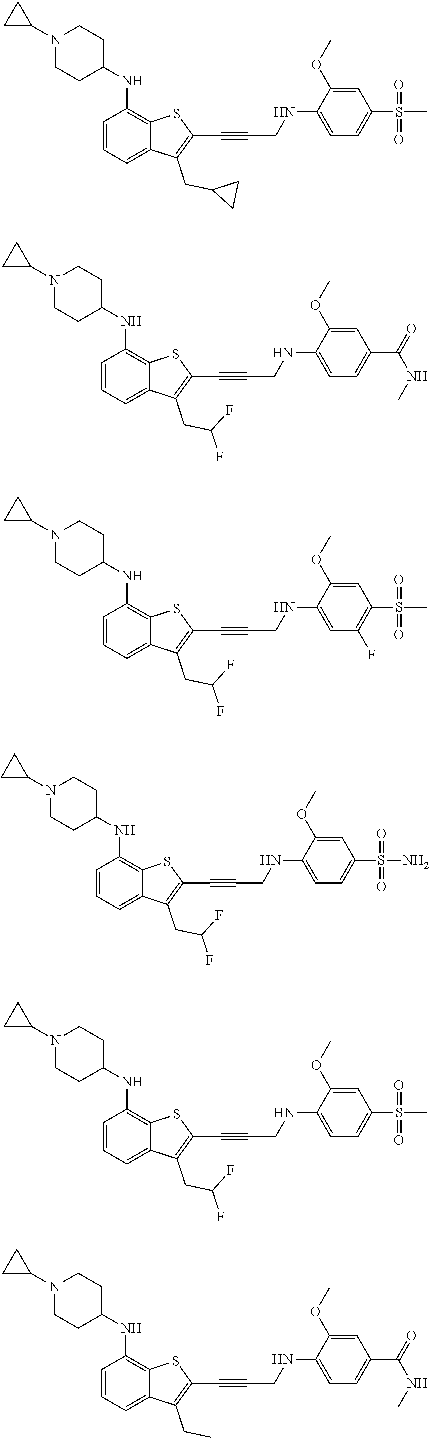 [SMILES: COc1cc(S(C)(=O)=O)ccc1NCC#Cc1sc2c(NC3CCN(C4CC4)CC3)cccc2c1CC1CC1 ; CNC(=O)c1ccc(NCC#Cc2sc3c(NC4CCN(C5CC5)CC4)cccc3c2CC(F)F)c(OC)c1 ; COc1cc(S(C)(=O)=O)c(F)cc1NCC#Cc1sc2c(NC3CCN(C4CC4)CC3)cccc2c1CC(F)F ; COc1cc(S(N)(=O)=O)ccc1NCC#Cc1sc2c(NC3CCN(C4CC4)CC3)cccc2c1CC(F)F ; COc1cc(S(C)(=O)=O)ccc1NCC#Cc1sc2c(NC3CCN(C4CC4)CC3)cccc2c1CC(F)F ; CCc1c(C#CCNc2ccc(C(=O)NC)cc2OC)sc2c(NC3CCN(C4CC4)CC3)cccc12]

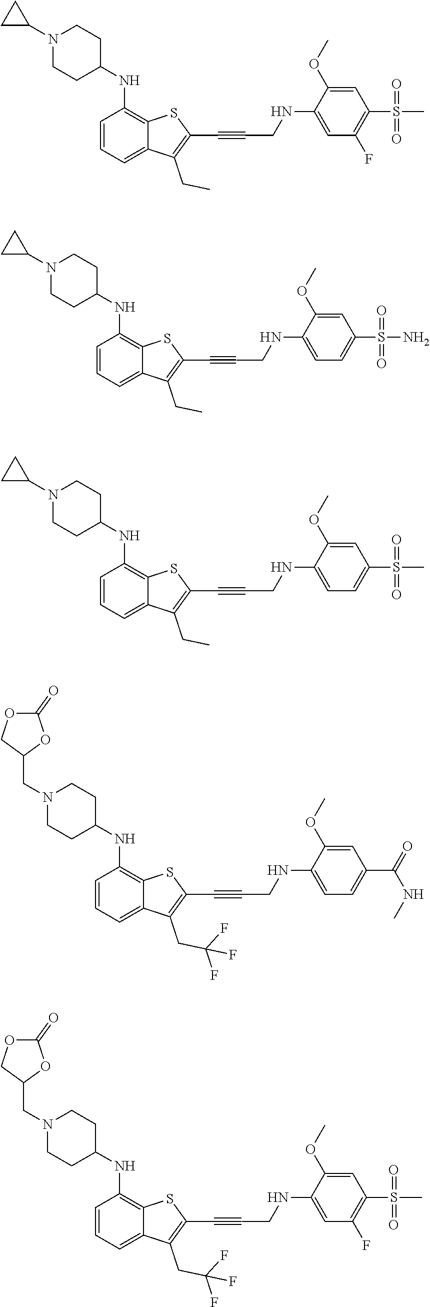 [SMILES: CCc1c(C#CCNc2cc(F)c(S(C)(=O)=O)cc2OC)sc2c(NC3CCN(C4CC4)CC3)cccc12 ; CCc1c(C#CCNc2ccc(S(N)(=O)=O)cc2OC)sc2c(NC3CCN(C4CC4)CC3)cccc12 ; CCc1c(C#CCNc2ccc(S(C)(=O)=O)cc2OC)sc2c(NC3CCN(C4CC4)CC3)cccc12 ; CNC(=O)c1ccc(NCC#Cc2sc3c(NC4CCN(CC5COC(=O)O5)CC4)cccc3c2CC(F)(F)F)c(OC)c1 ; COc1cc(S(C)(=O)=O)c(F)cc1NCC#Cc1sc2c(NC3CCN(CC4COC(=O)O4)CC3)cccc2c1CC(F)(F)F]

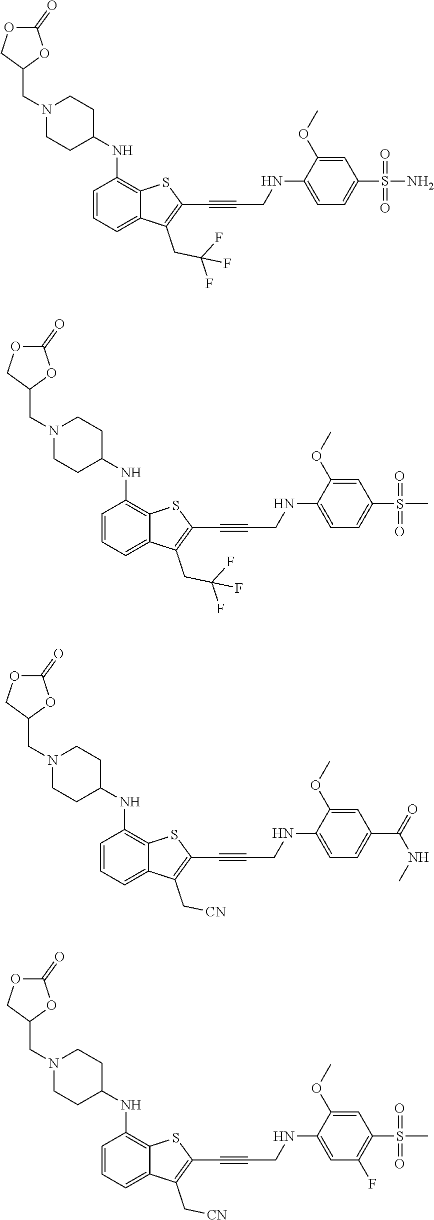 [SMILES: COc1cc(S(N)(=O)=O)ccc1NCC#Cc1sc2c(NC3CCN(CC4COC(=O)O4)CC3)cccc2c1CC(F)(F)F ; COc1cc(S(C)(=O)=O)ccc1NCC#Cc1sc2c(NC3CCN(CC4COC(=O)O4)CC3)cccc2c1CC(F)(F)F ; CNC(=O)c1ccc(NCC#Cc2sc3c(NC4CCN(CC5COC(=O)O5)CC4)cccc3c2CC#N)c(OC)c1 ; COc1cc(S(C)(=O)=O)c(F)cc1NCC#Cc1sc2c(NC3CCN(CC4COC(=O)O4)CC3)cccc2c1CC#N]

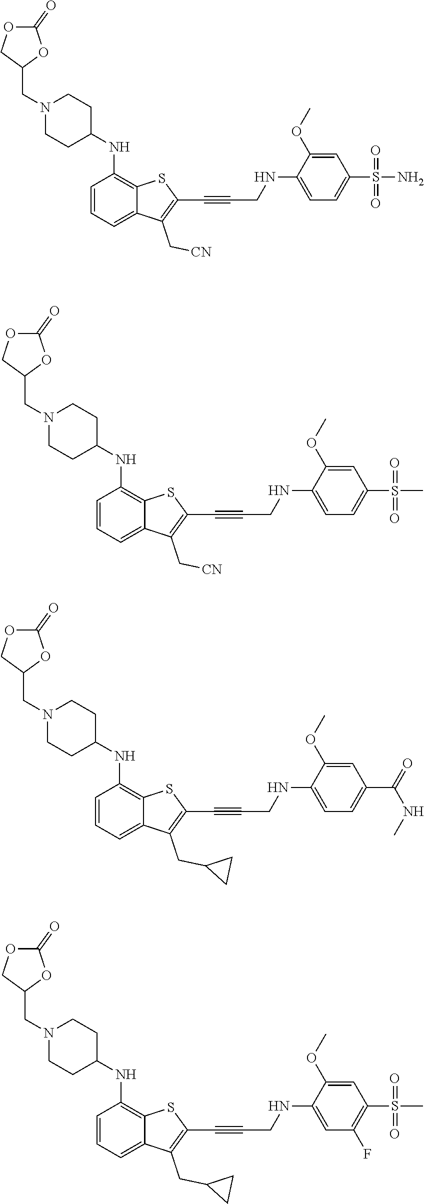 [SMILES: COc1cc(S(N)(=O)=O)ccc1NCC#Cc1sc2c(NC3CCN(CC4COC(=O)O4)CC3)cccc2c1CC#N ; COc1cc(S(C)(=O)=O)ccc1NCC#Cc1sc2c(NC3CCN(CC4COC(=O)O4)CC3)cccc2c1CC#N ; CNC(=O)c1ccc(NCC#Cc2sc3c(NC4CCN(CC5COC(=O)O5)CC4)cccc3c2CC2CC2)c(OC)c1 ; COc1cc(S(C)(=O)=O)c(F)cc1NCC#Cc1sc2c(NC3CCN(CC4COC(=O)O4)CC3)cccc2c1CC1CC1]

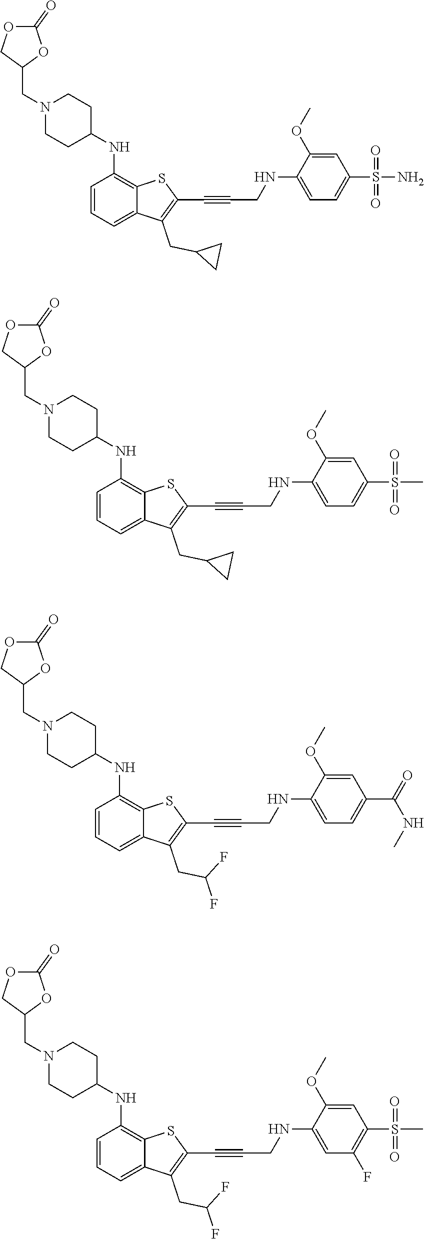 [SMILES: COc1cc(S(N)(=O)=O)ccc1NCC#Cc1sc2c(NC3CCN(CC4COC(=O)O4)CC3)cccc2c1CC1CC1 ; COc1cc(S(C)(=O)=O)ccc1NCC#Cc1sc2c(NC3CCN(CC4COC(=O)O4)CC3)cccc2c1CC1CC1 ; CNC(=O)c1ccc(NCC#Cc2sc3c(NC4CCN(CC5COC(=O)O5)CC4)cccc3c2CC(F)F)c(OC)c1 ; COc1cc(S(C)(=O)=O)c(F)cc1NCC#Cc1sc2c(NC3CCN(CC4COC(=O)O4)CC3)cccc2c1CC(F)F]

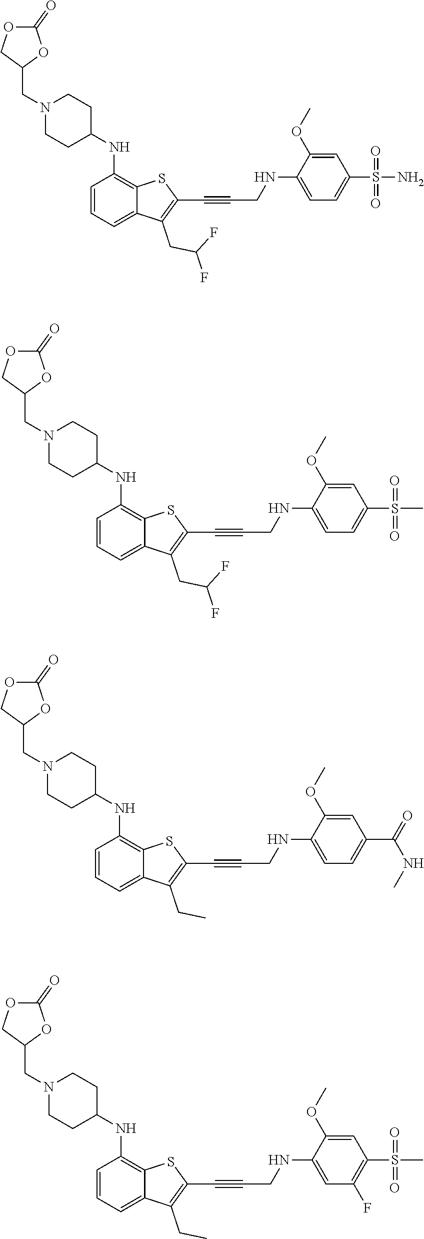 [SMILES: COc1cc(S(N)(=O)=O)ccc1NCC#Cc1sc2c(NC3CCN(CC4COC(=O)O4)CC3)cccc2c1CC(F)F ; COc1cc(S(C)(=O)=O)ccc1NCC#Cc1sc2c(NC3CCN(CC4COC(=O)O4)CC3)cccc2c1CC(F)F ; CCc1c(C#CCNc2ccc(C(=O)NC)cc2OC)sc2c(NC3CCN(CC4COC(=O)O4)CC3)cccc12 ; CCc1c(C#CCNc2cc(F)c(S(C)(=O)=O)cc2OC)sc2c(NC3CCN(CC4COC(=O)O4)CC3)cccc12]

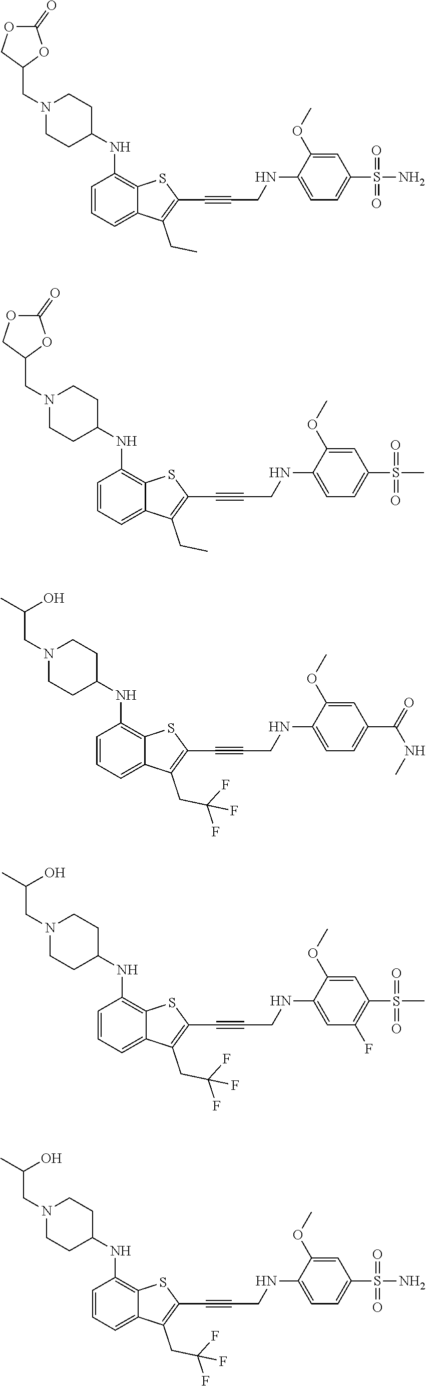

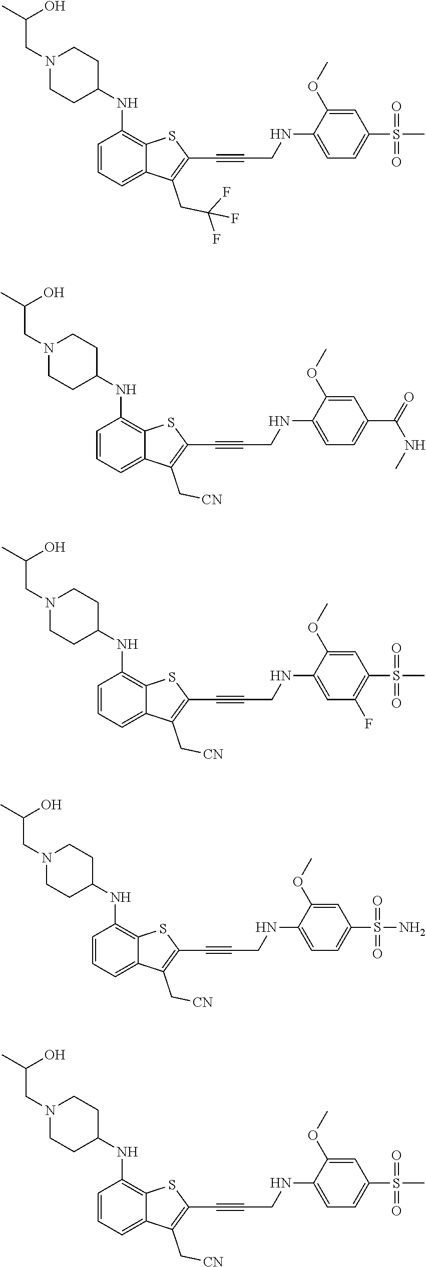

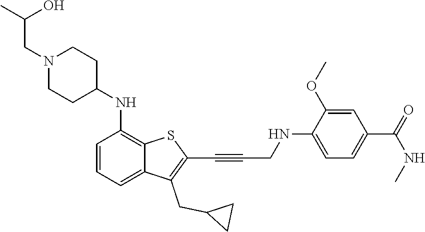 [SMILES: CNC(=O)c1ccc(NCC#Cc2sc3c(NC4CCN(CC(C)O)CC4)cccc3c2CC2CC2)c(OC)c1]

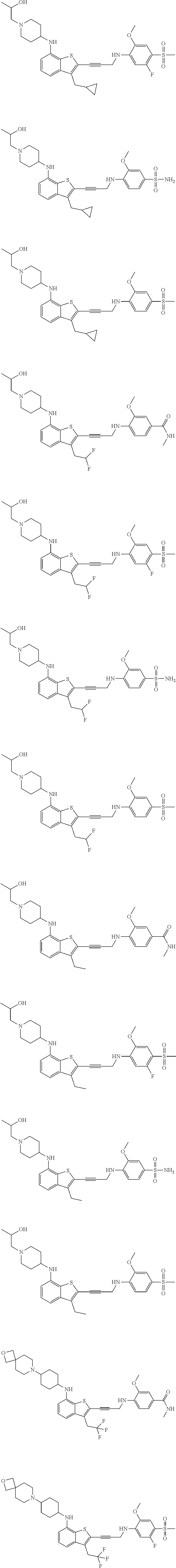

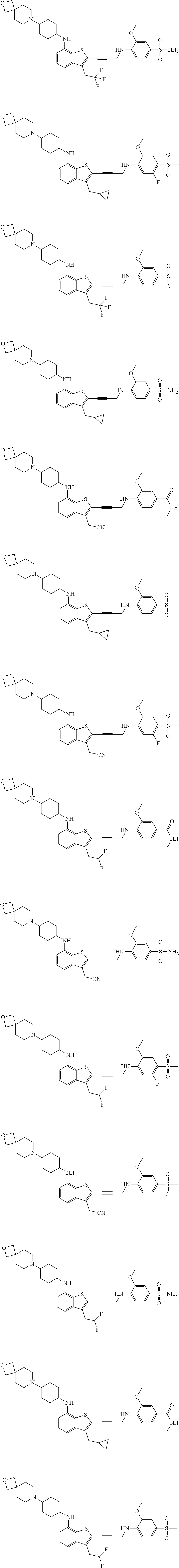

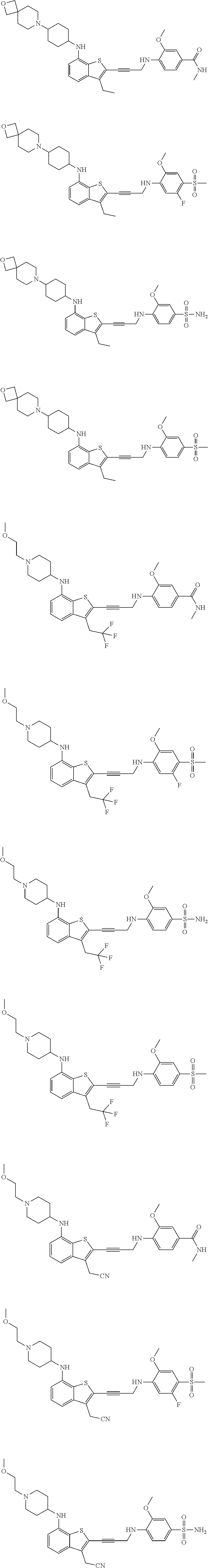

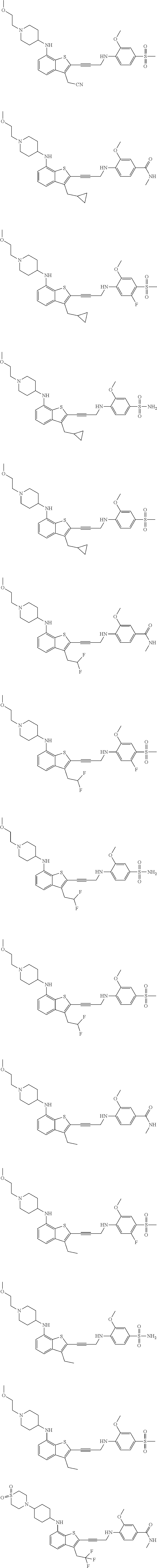

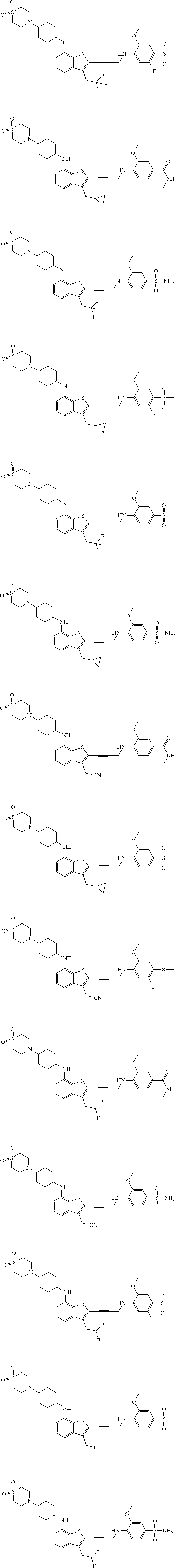

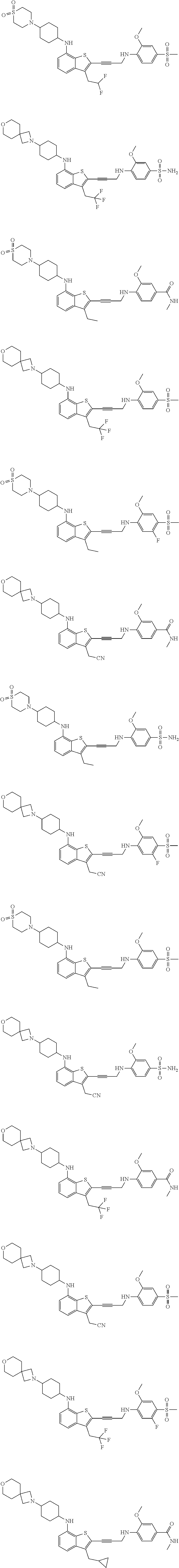

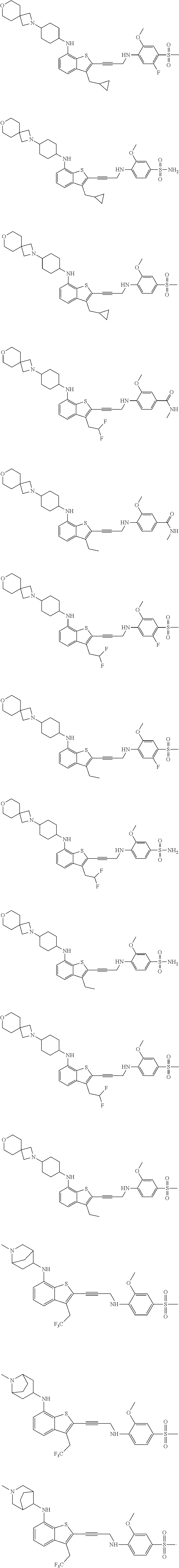

[58]. The compound according to any one of [1] to [53], wherein the compound of formula (I) is selected from:

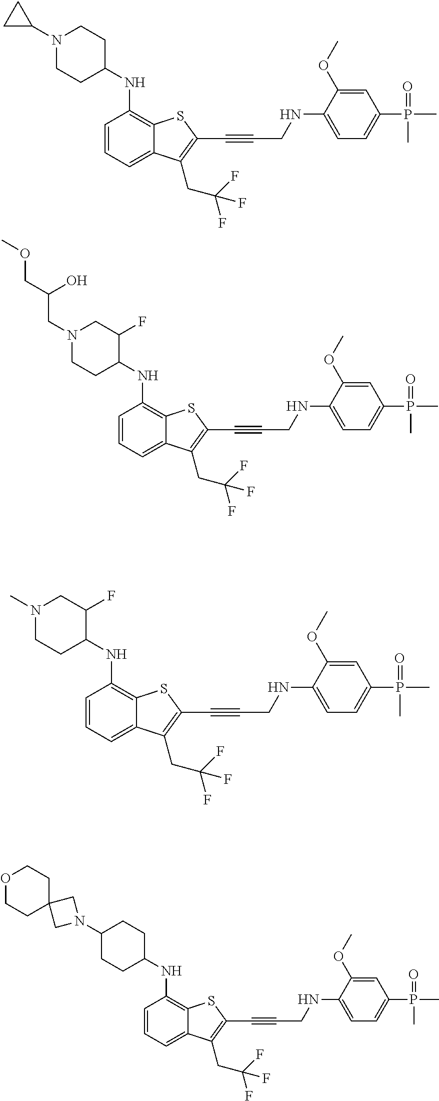

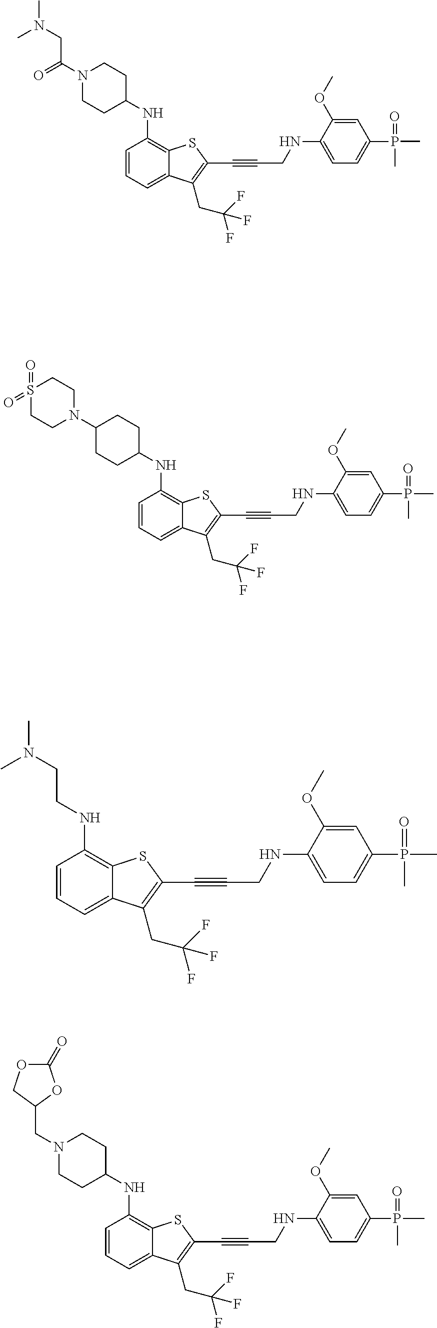

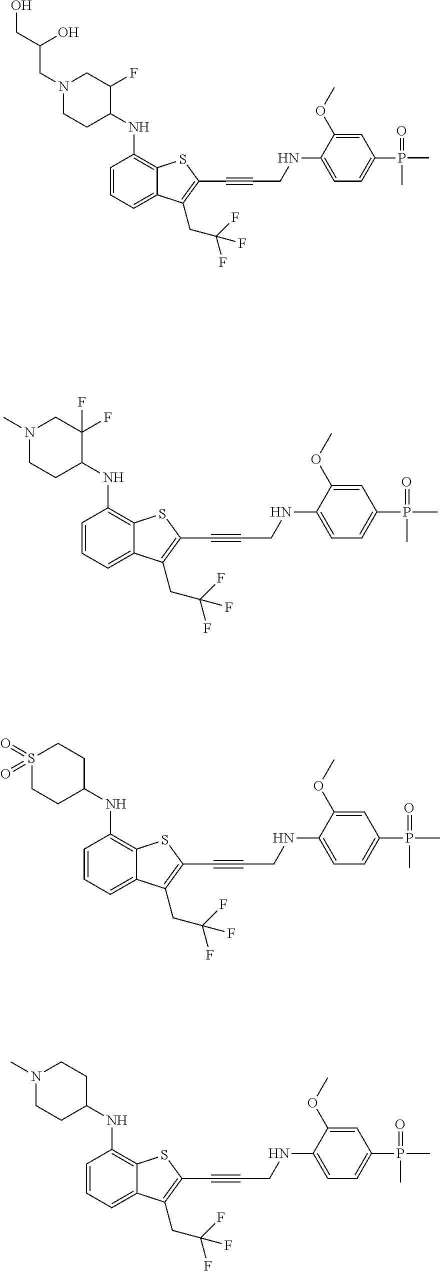

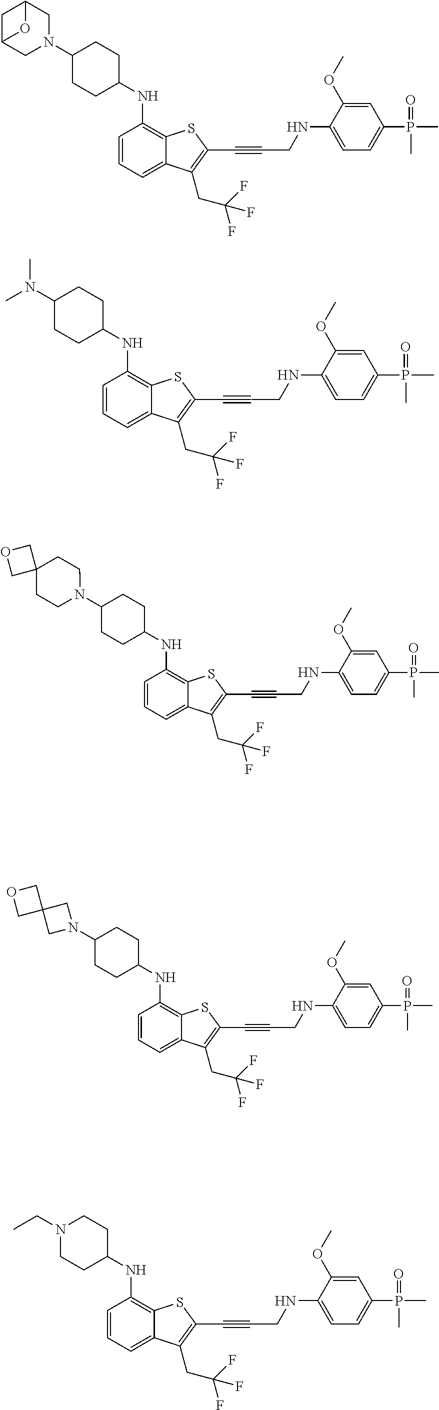

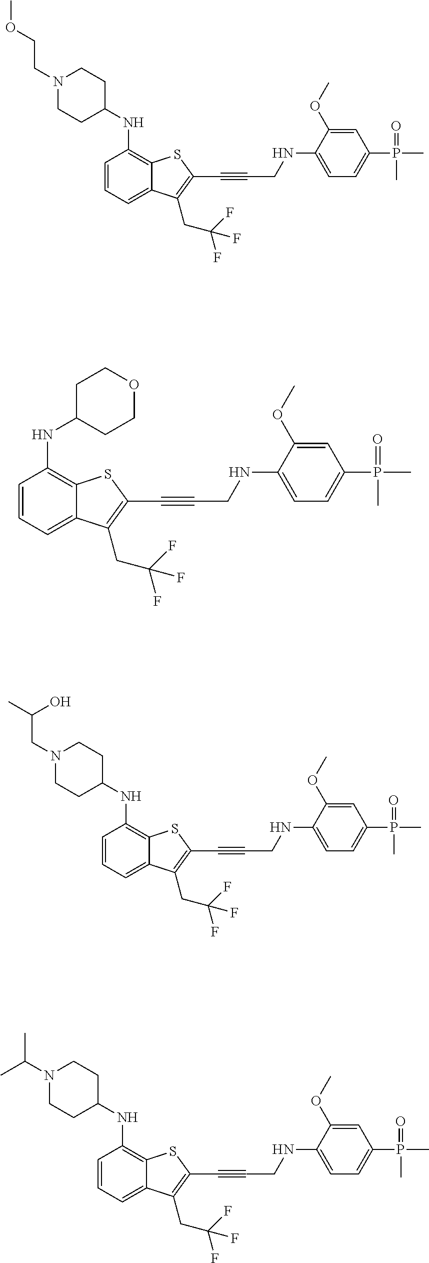

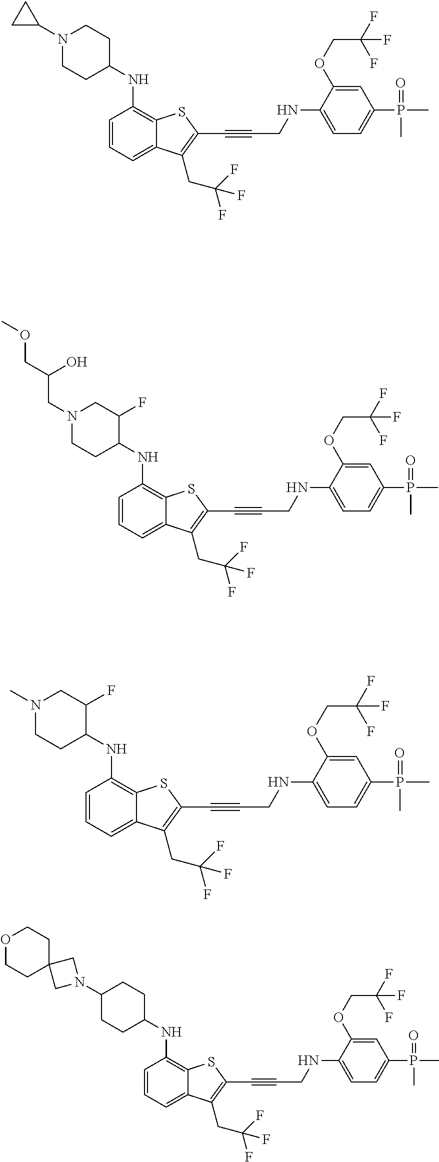

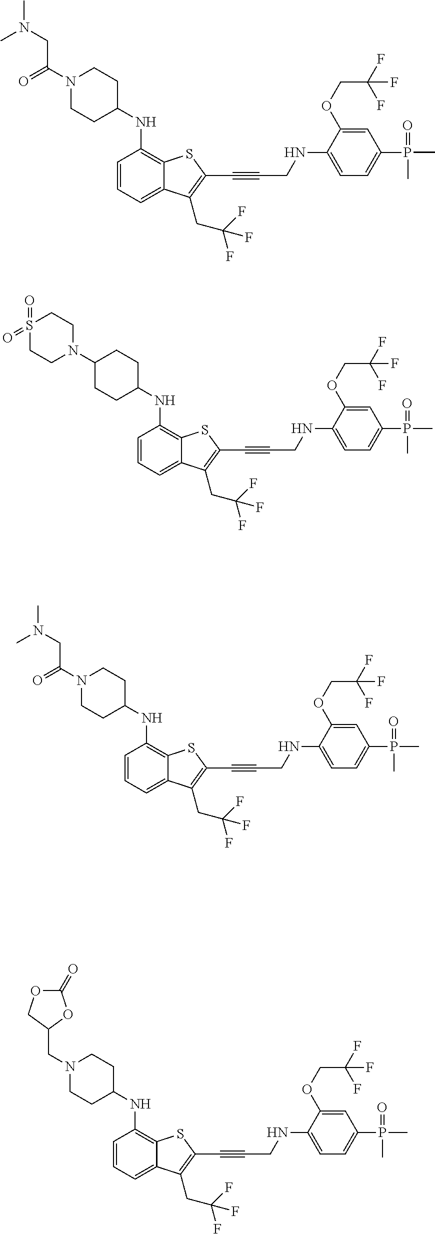

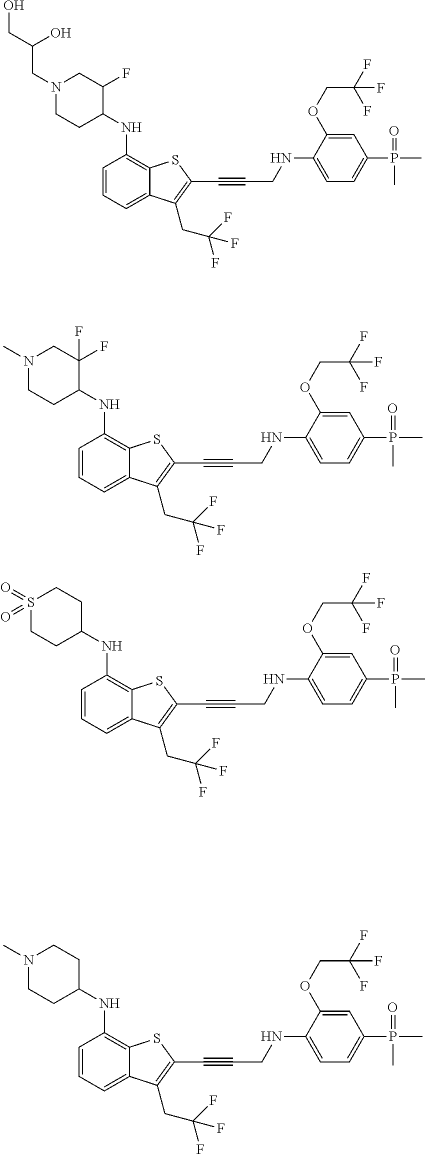

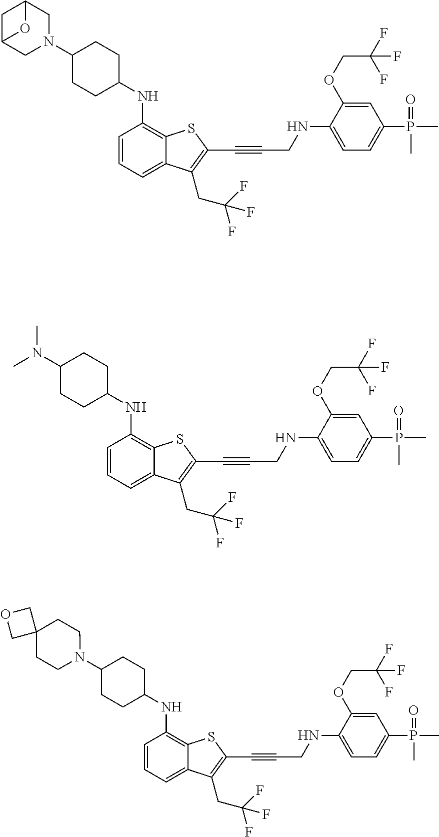

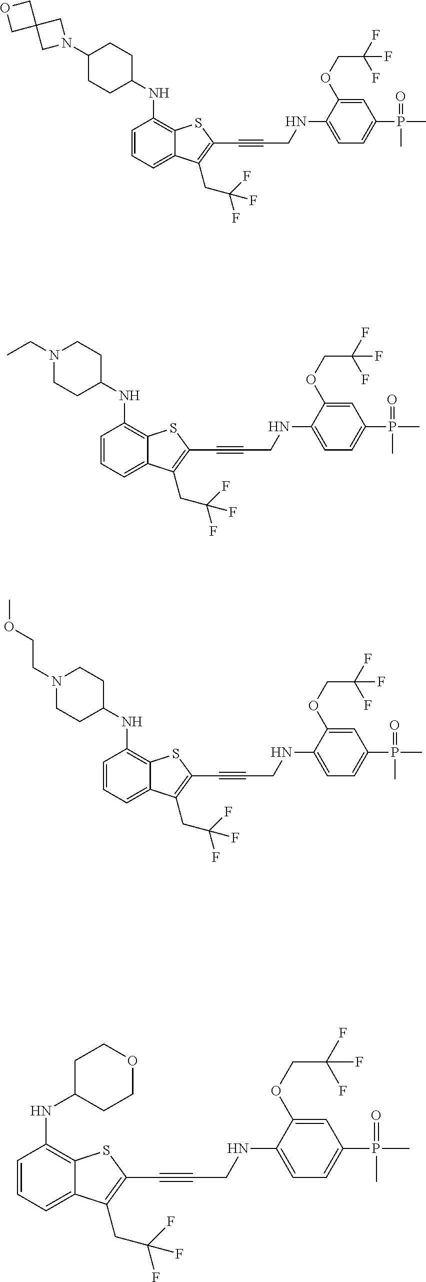

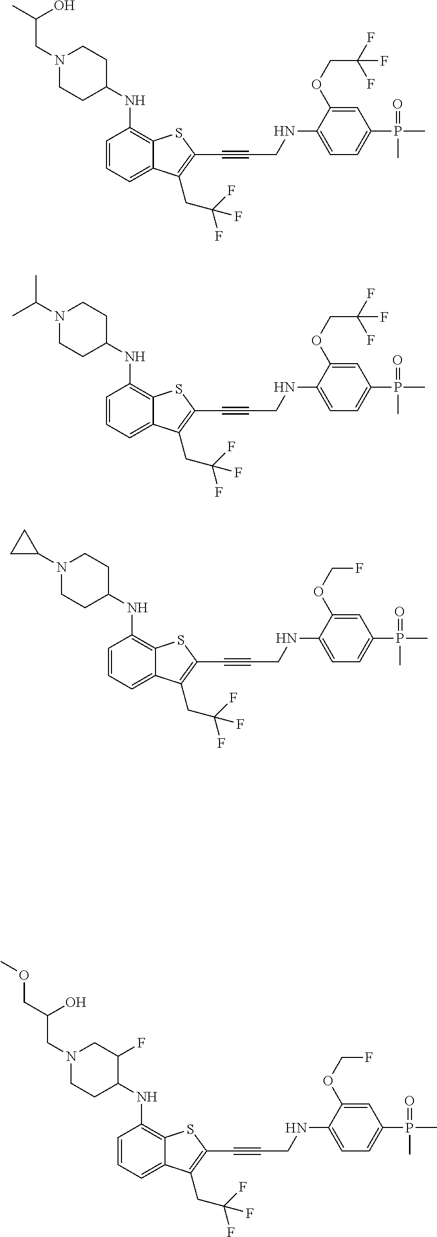

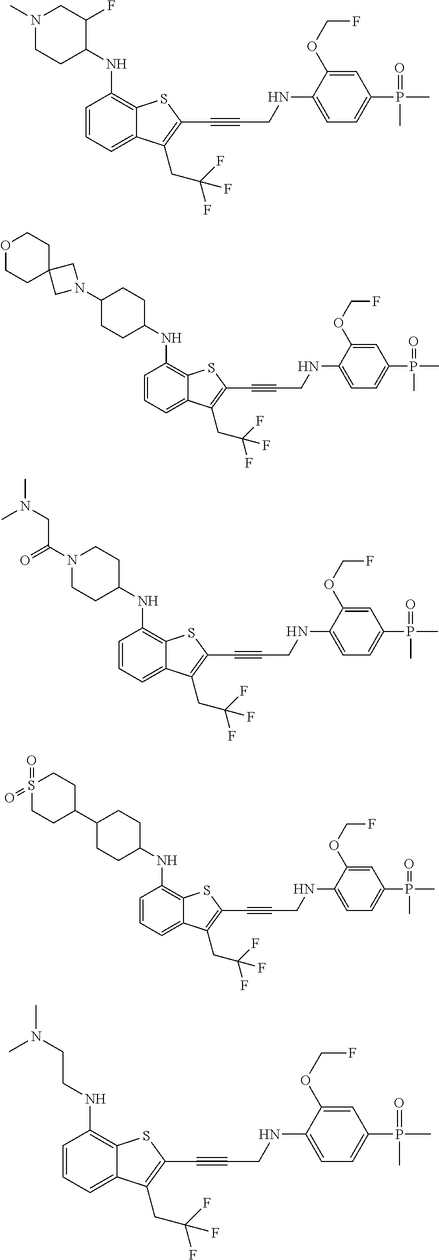

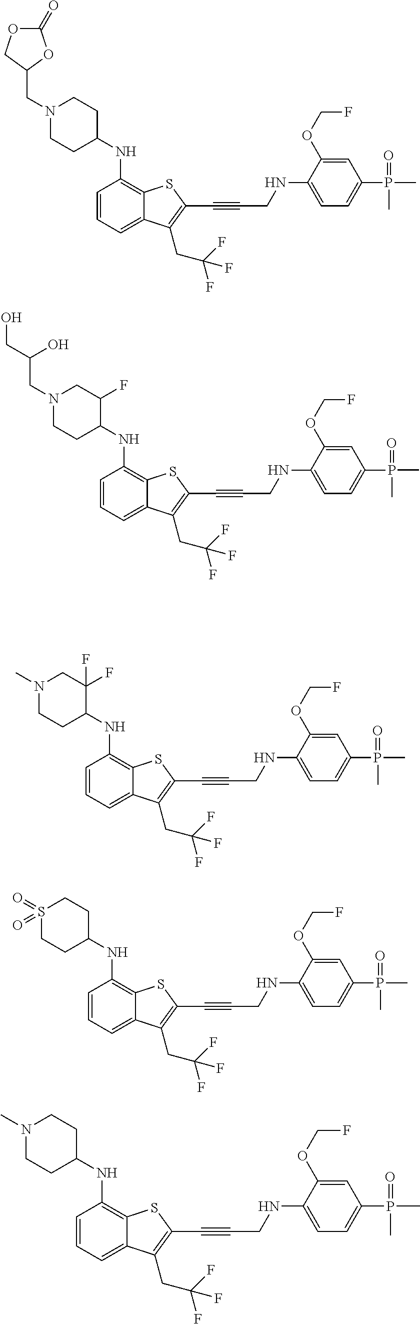

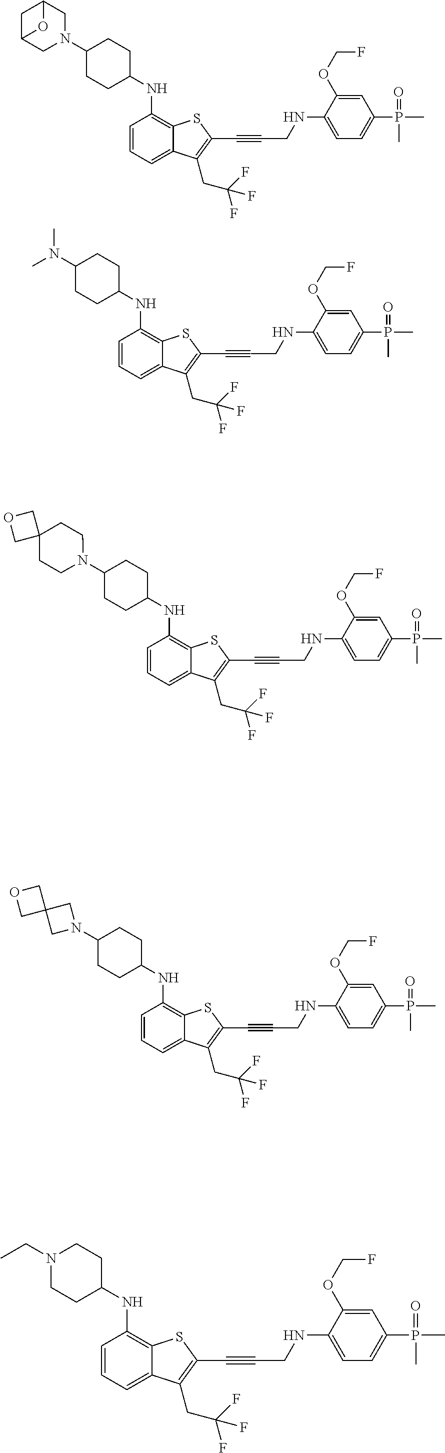

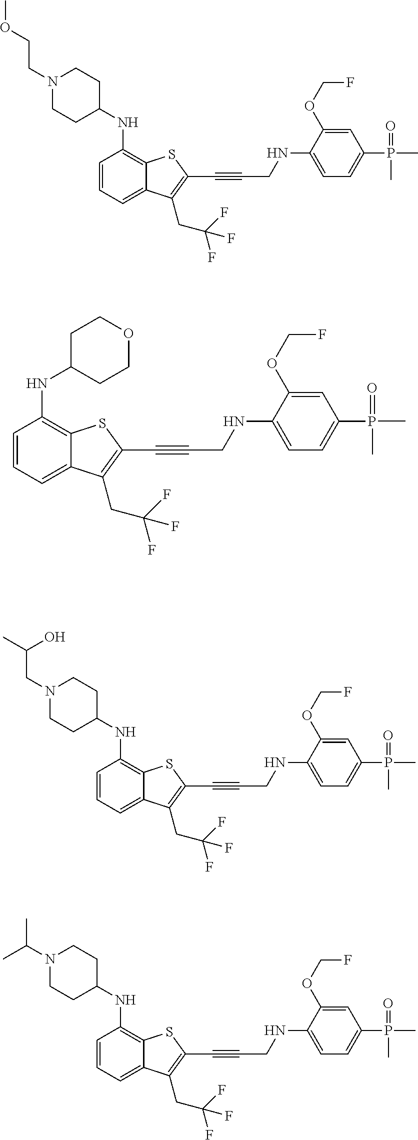

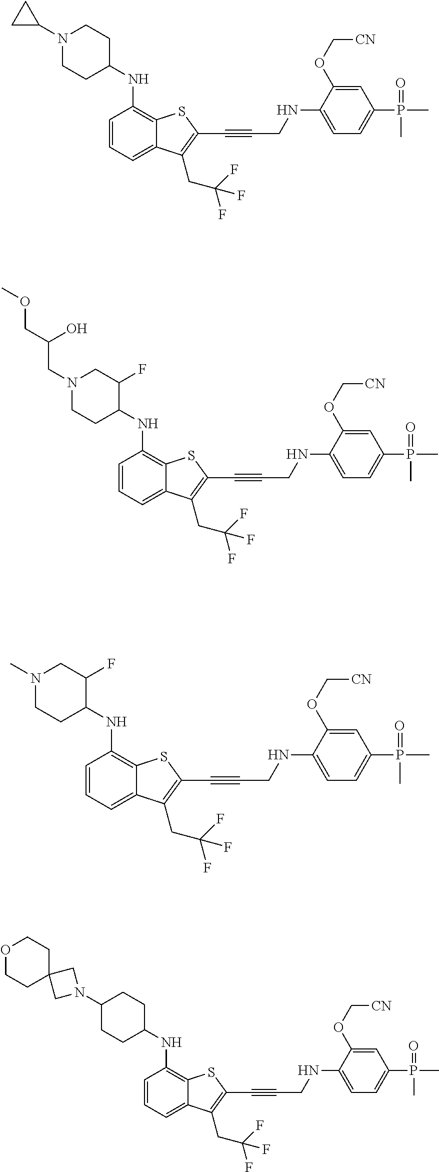

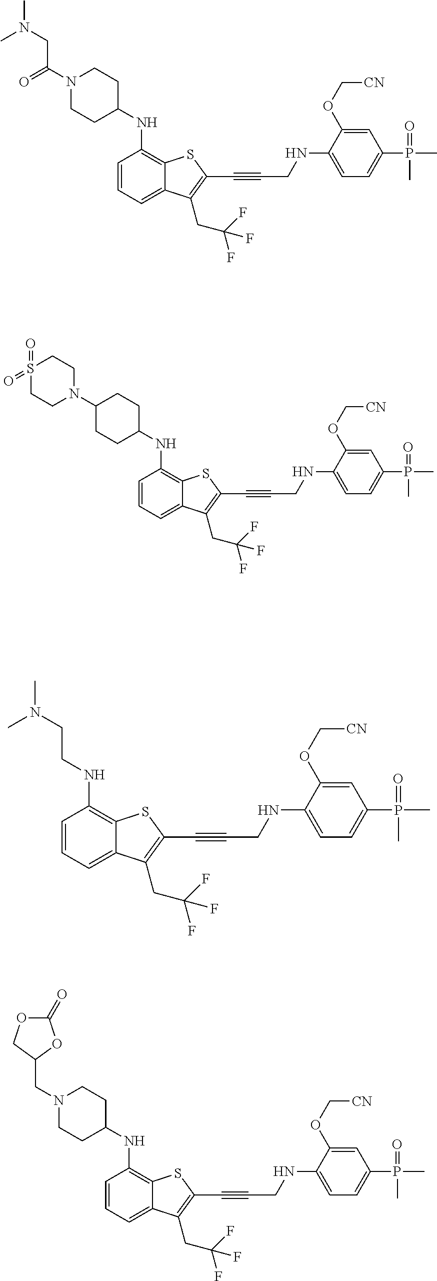

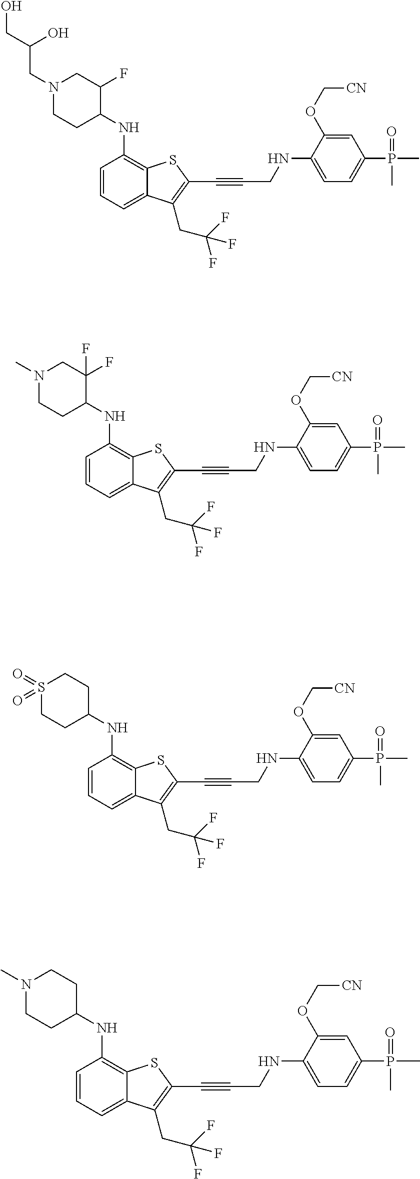

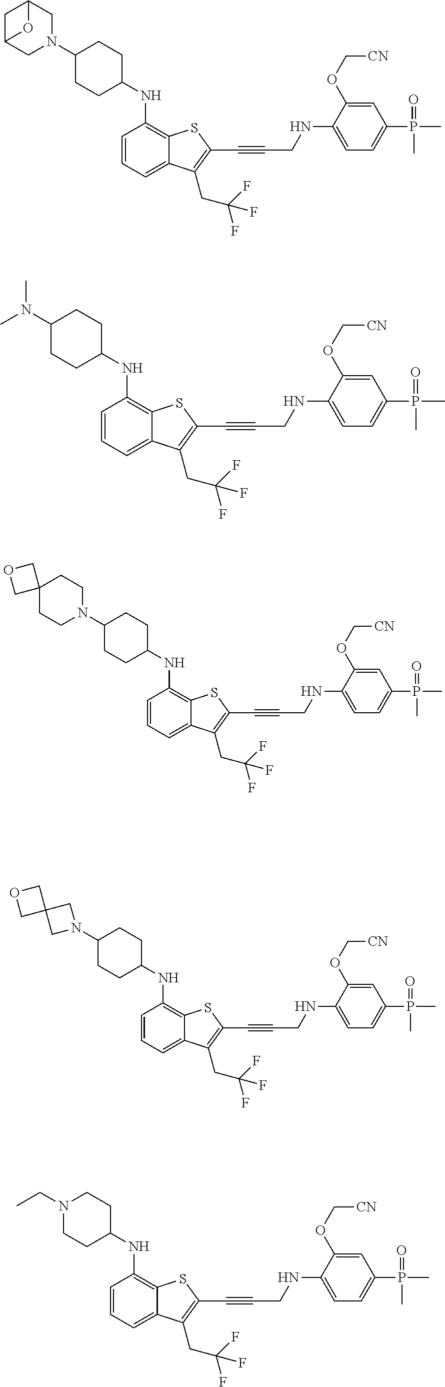

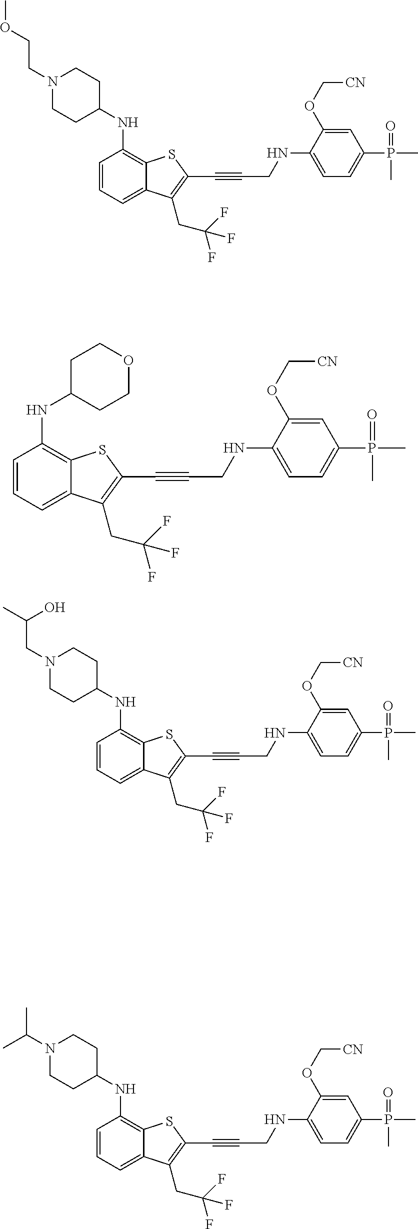

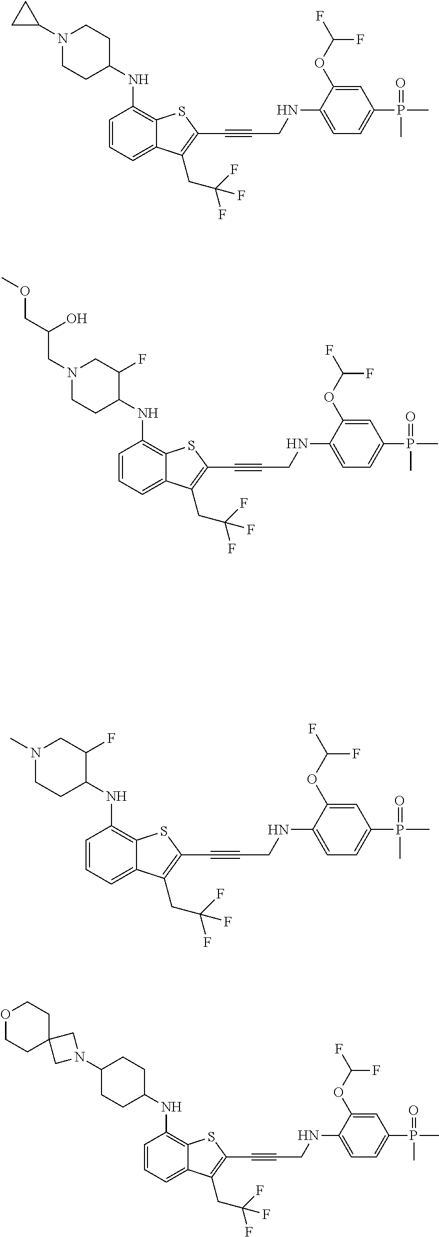

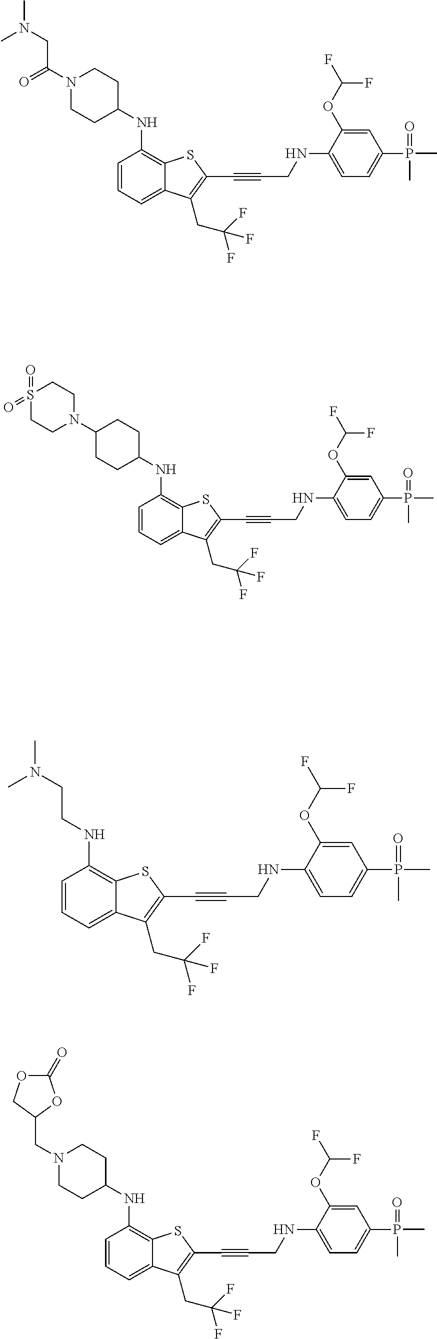

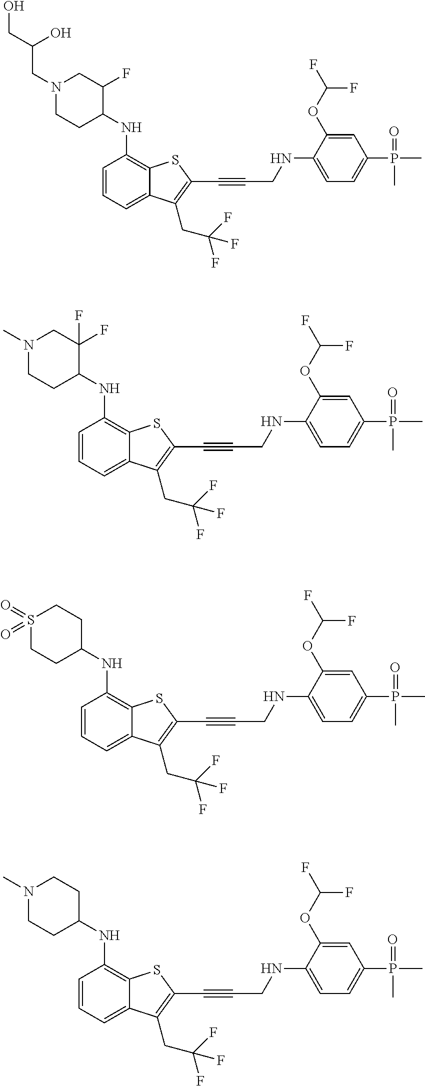

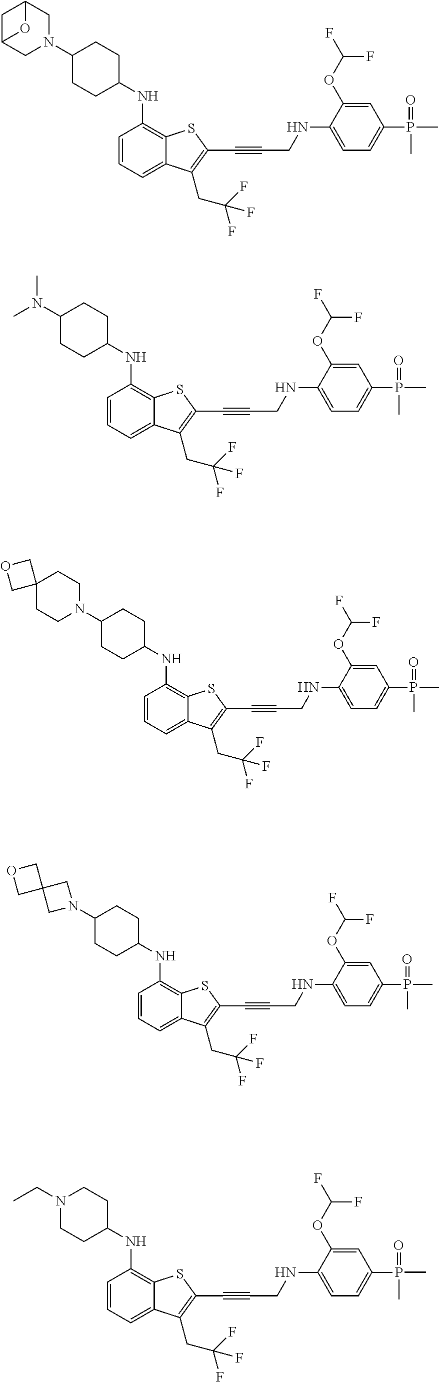

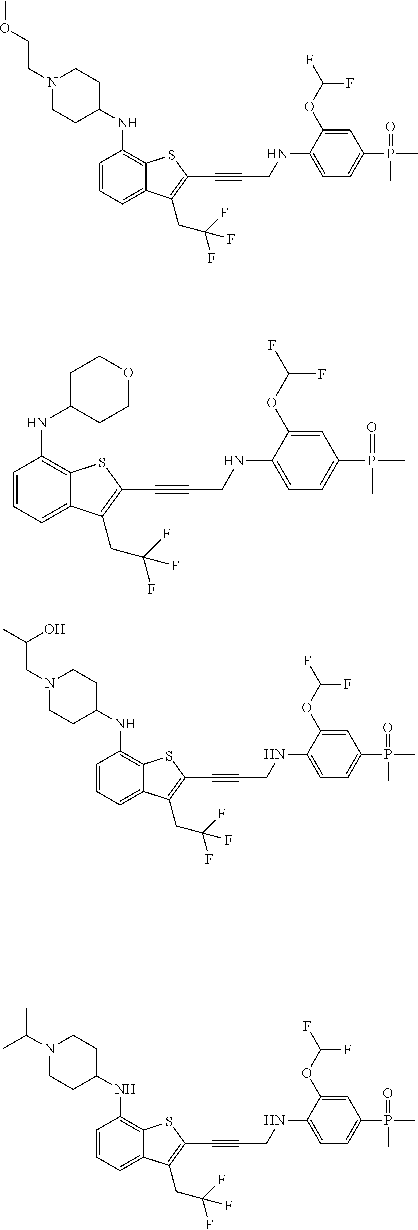

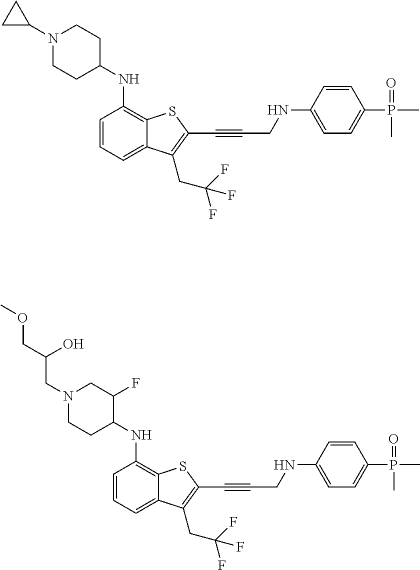

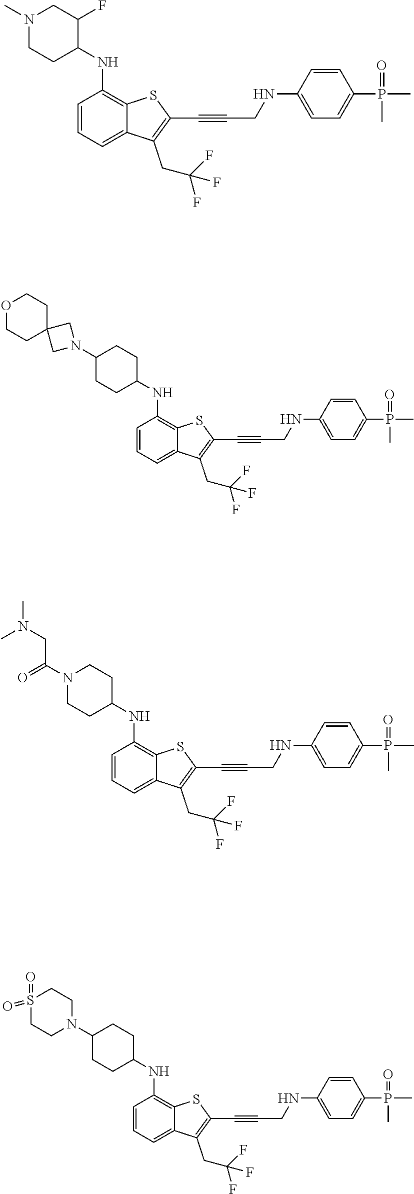

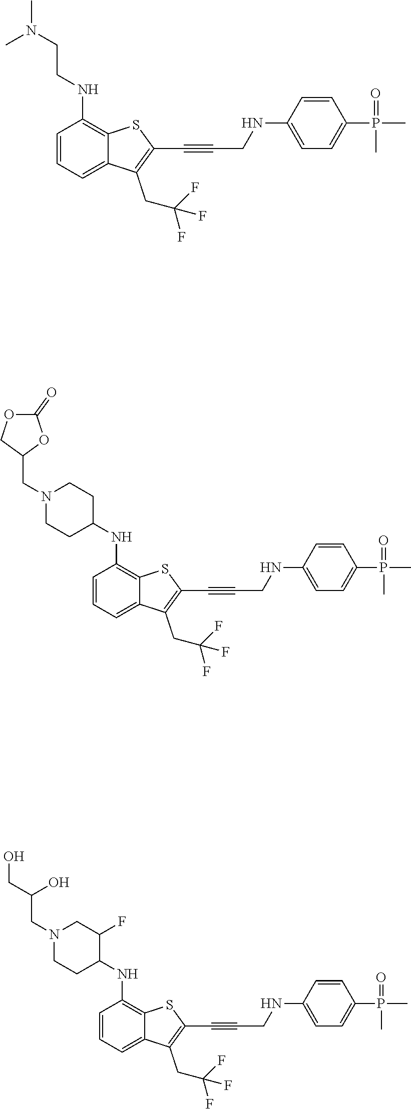

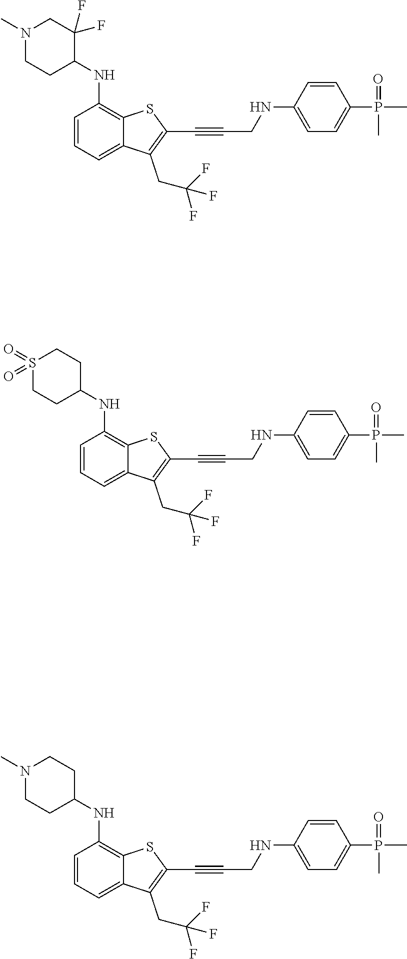

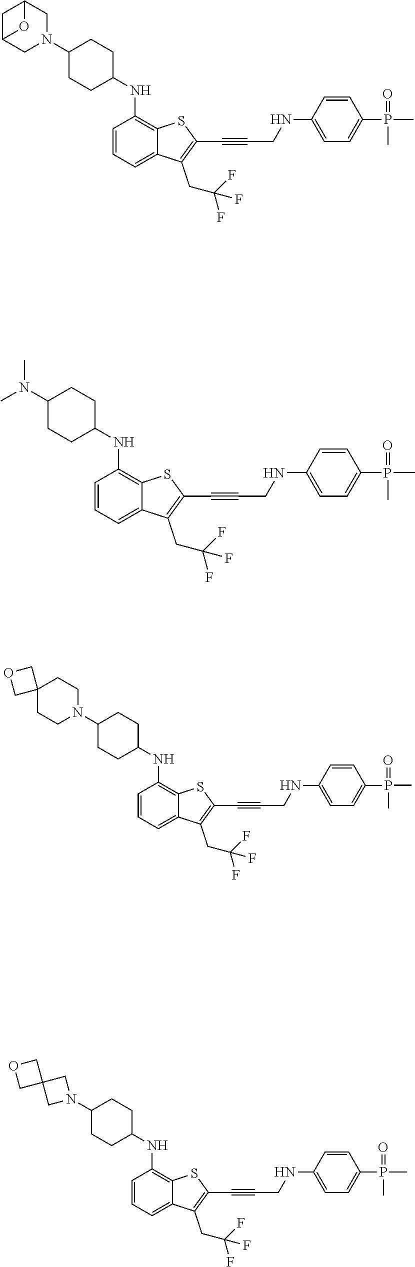

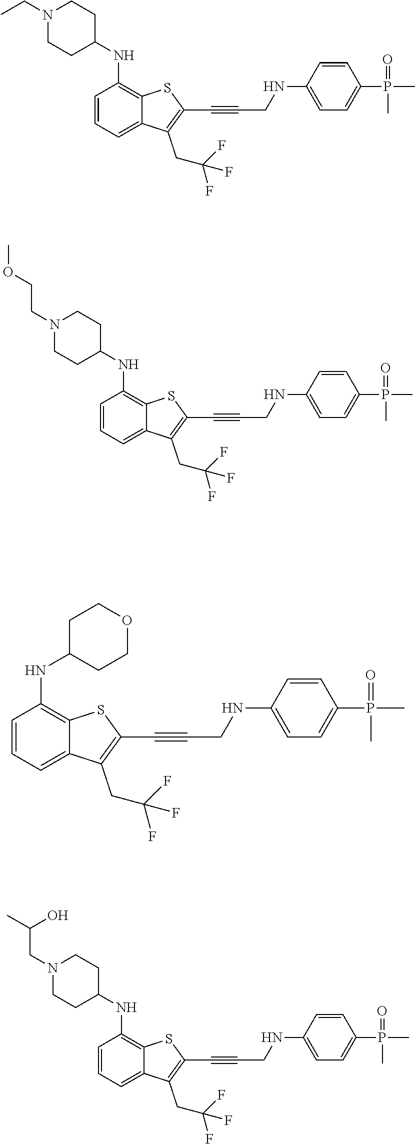

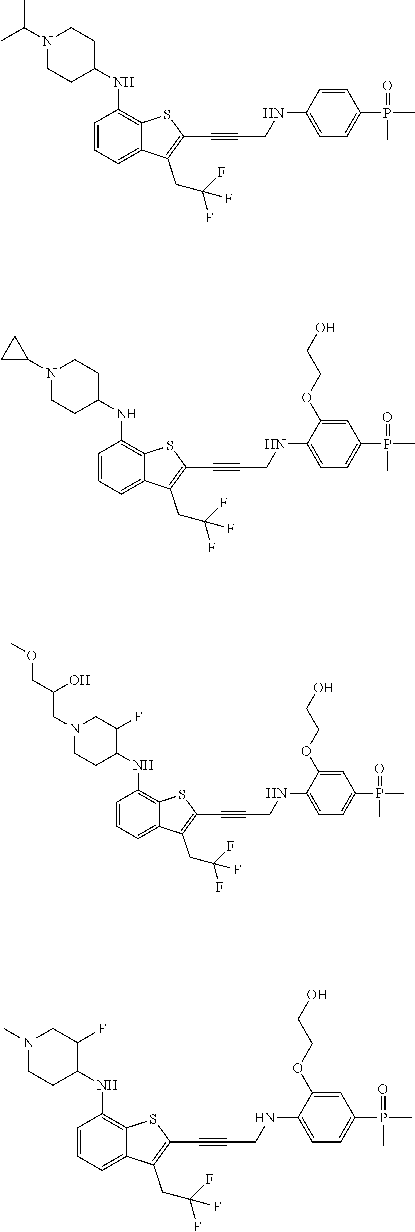

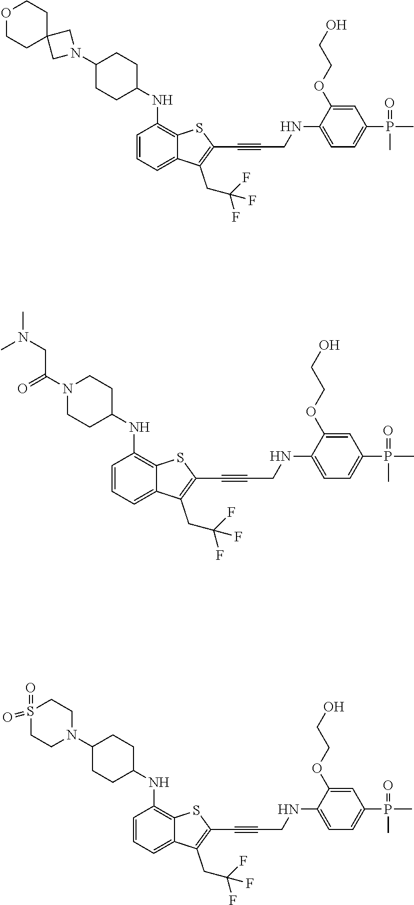

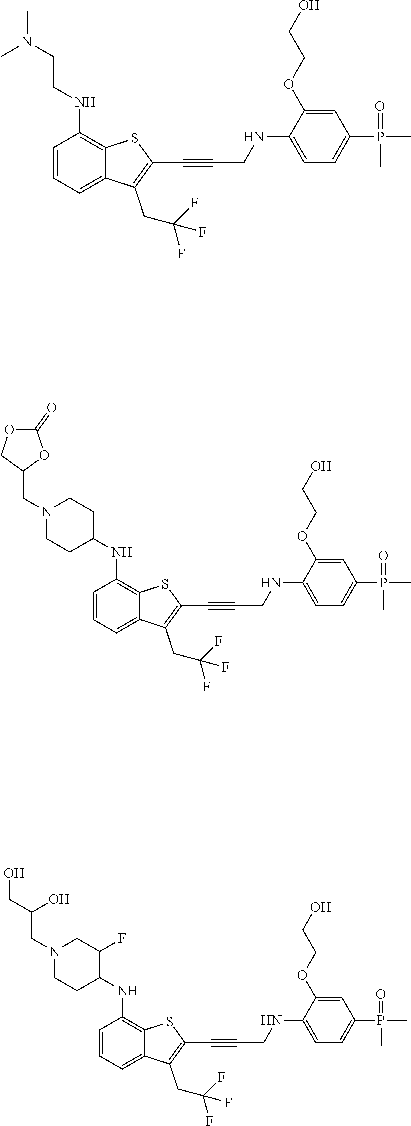

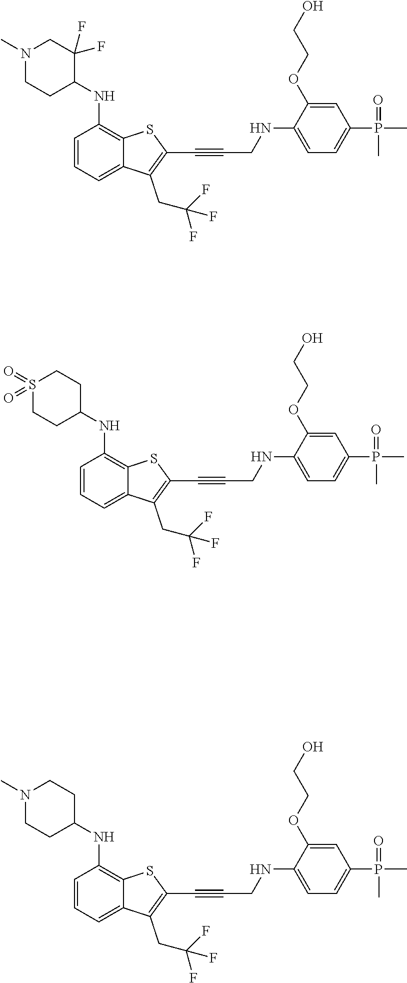

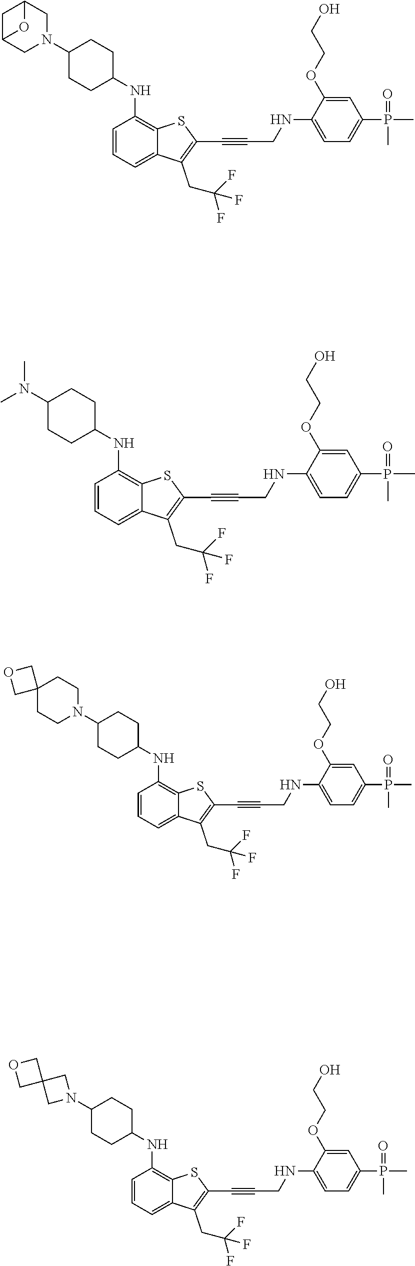

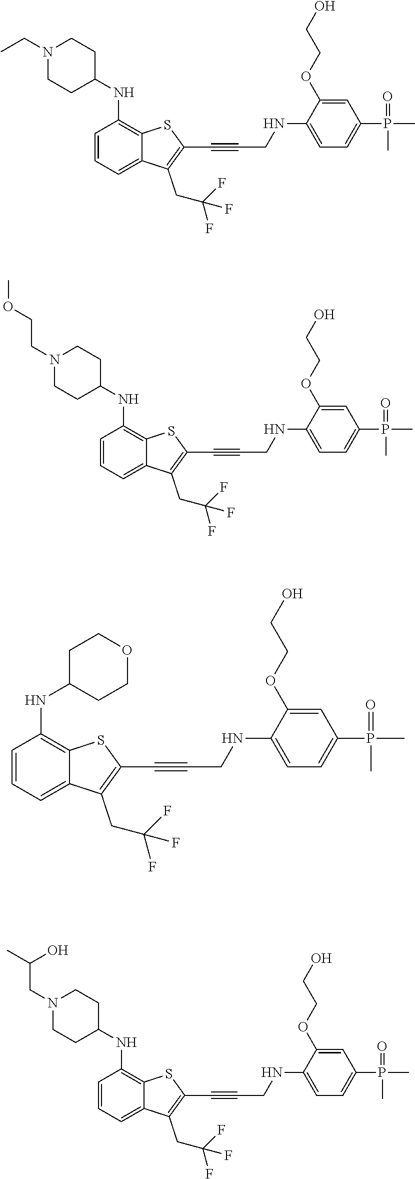

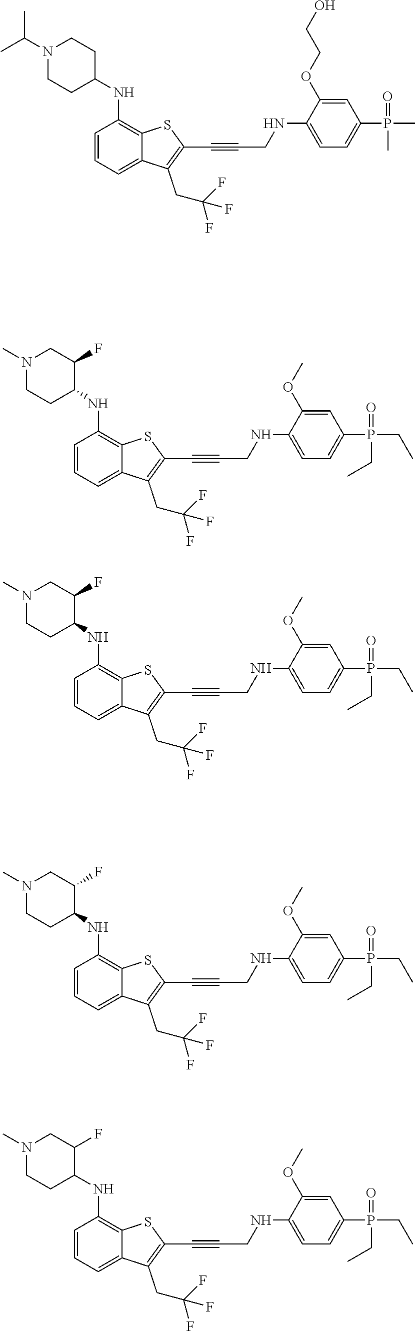

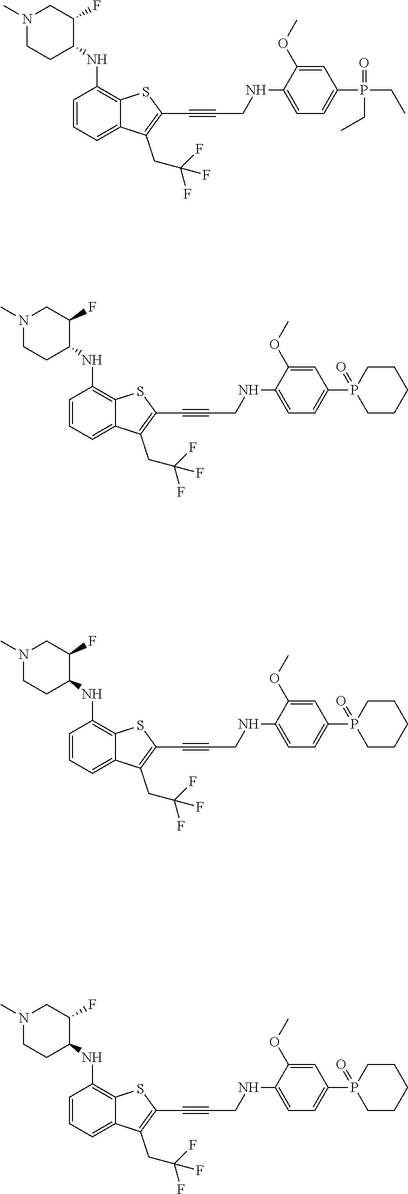

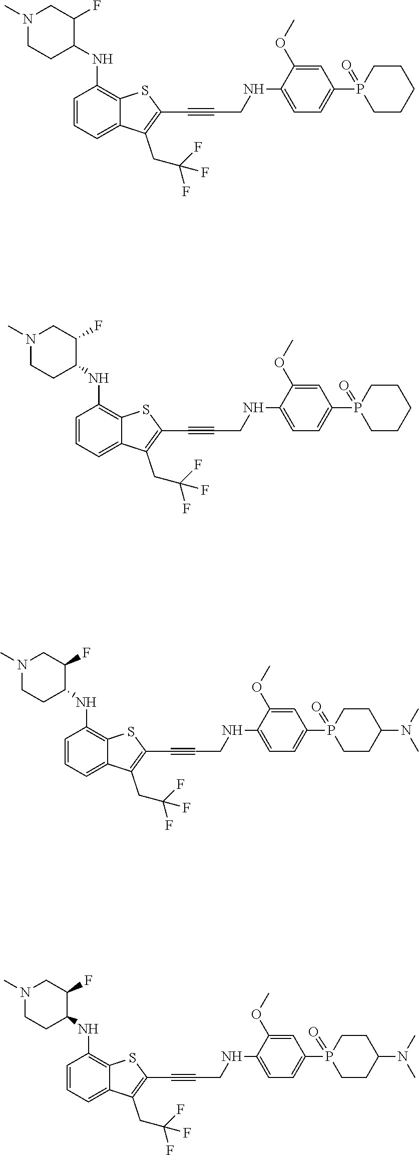

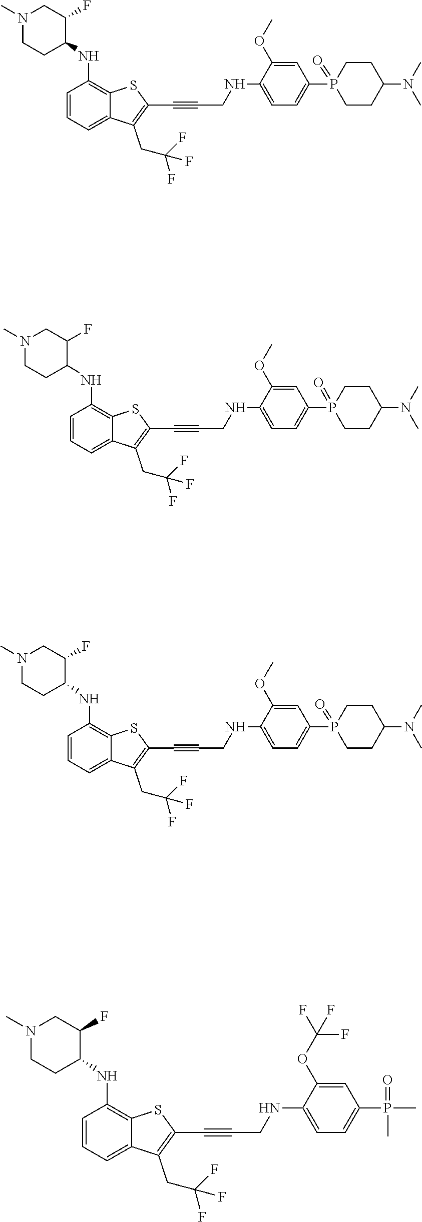

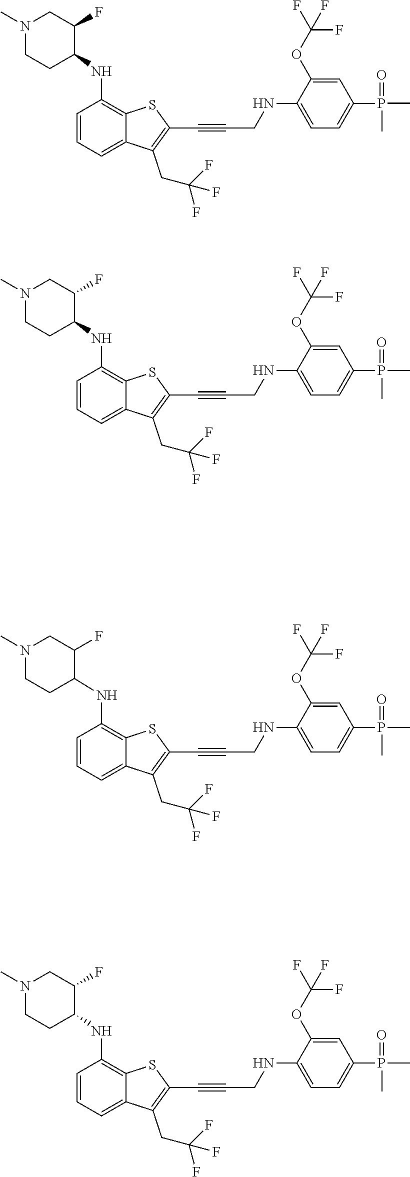

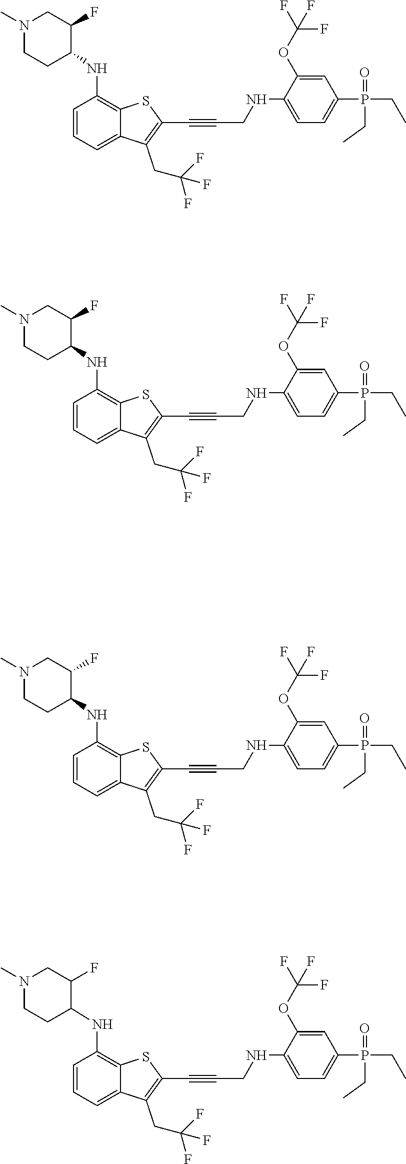

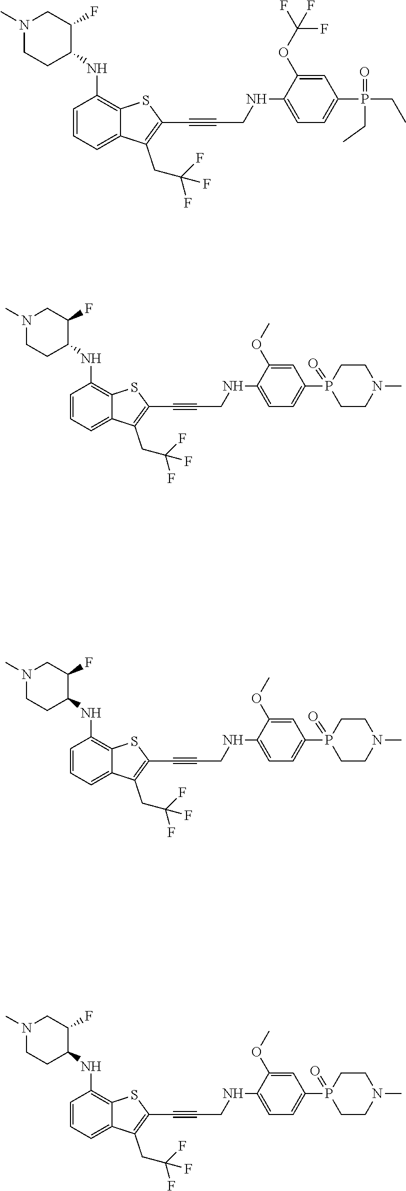

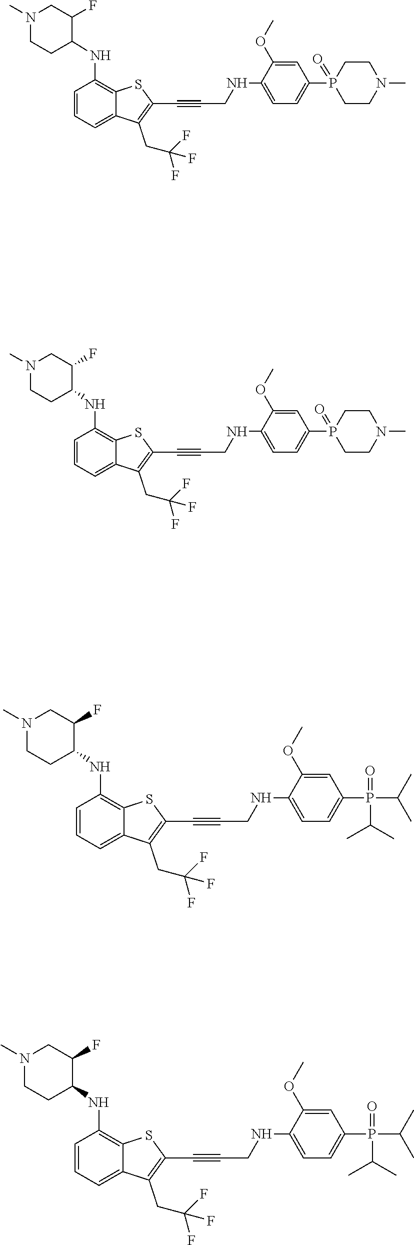

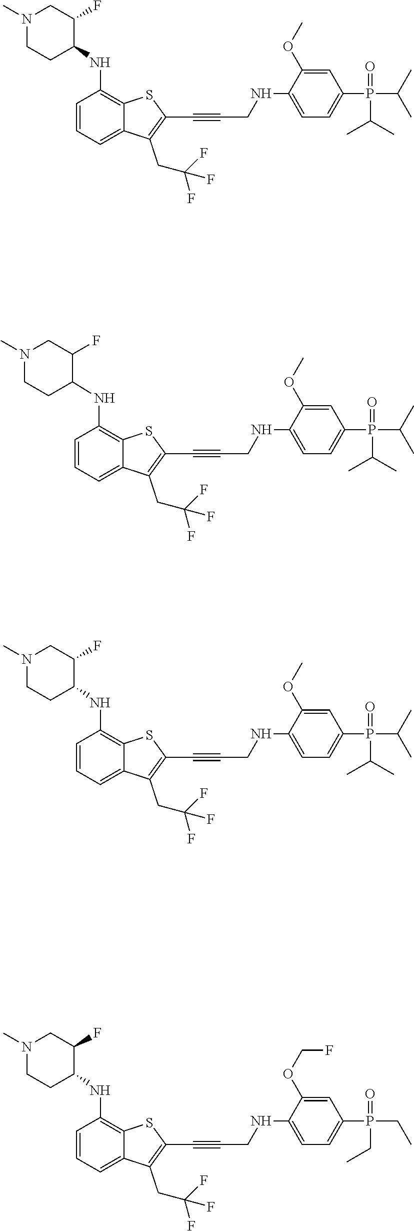

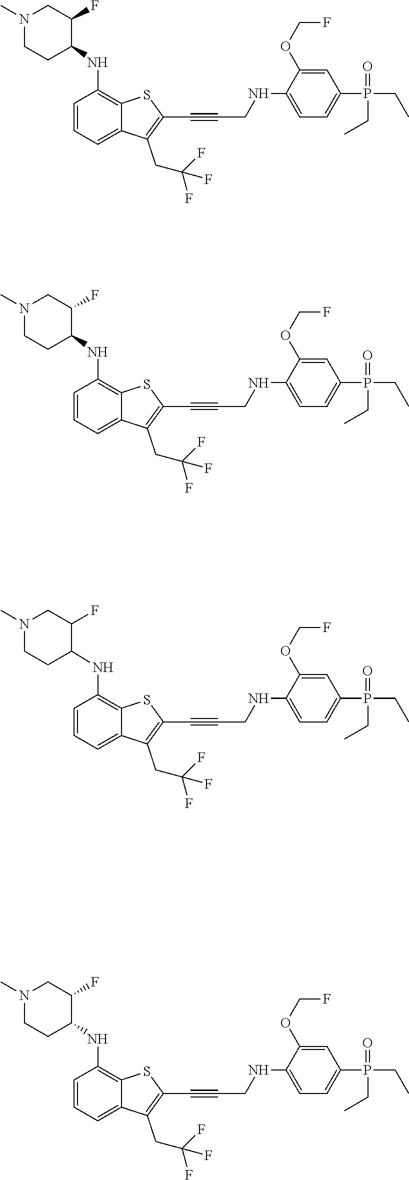

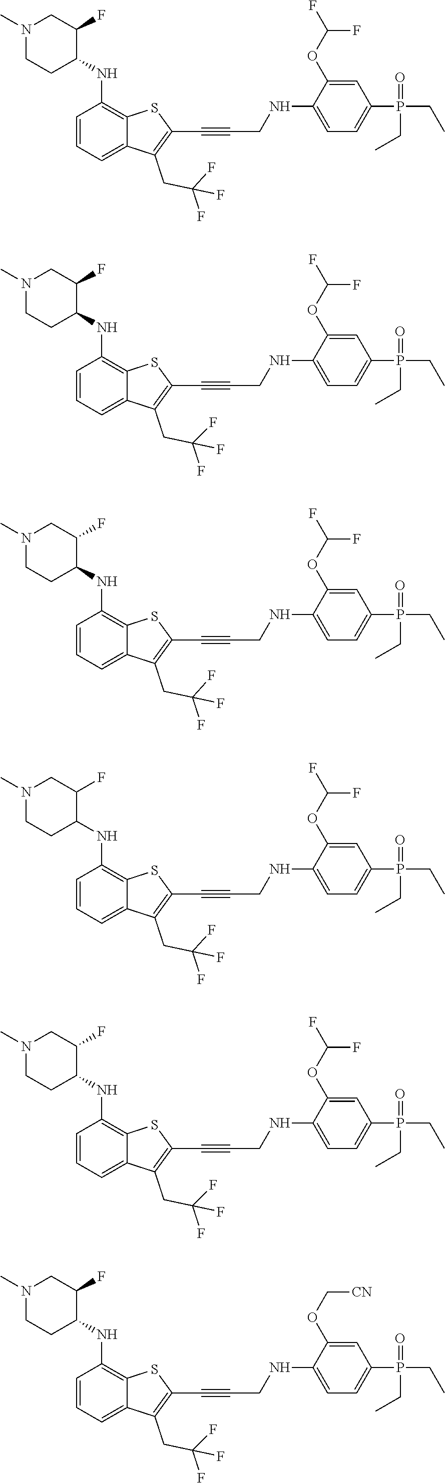

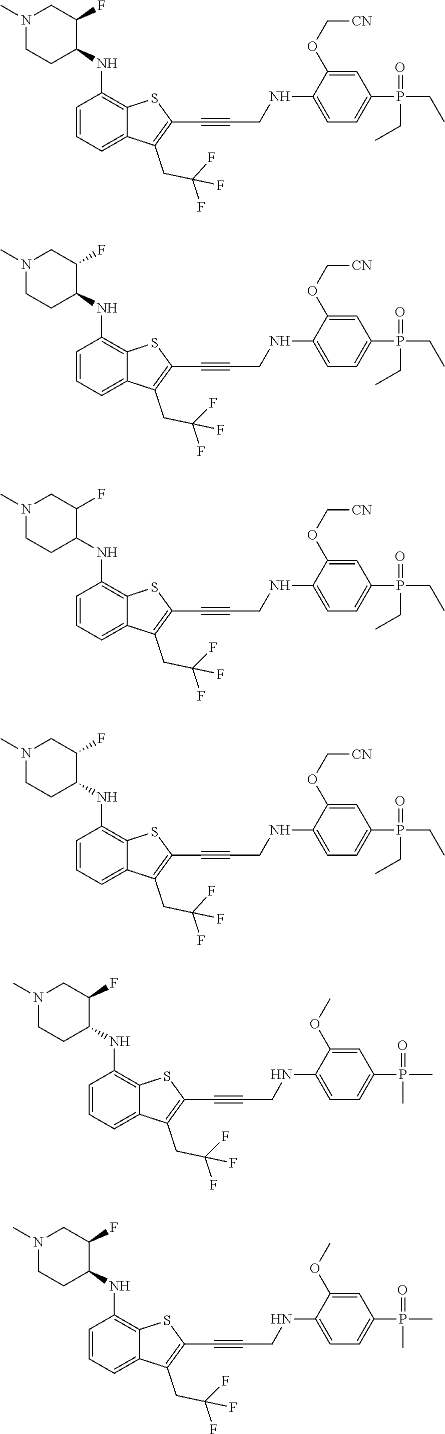

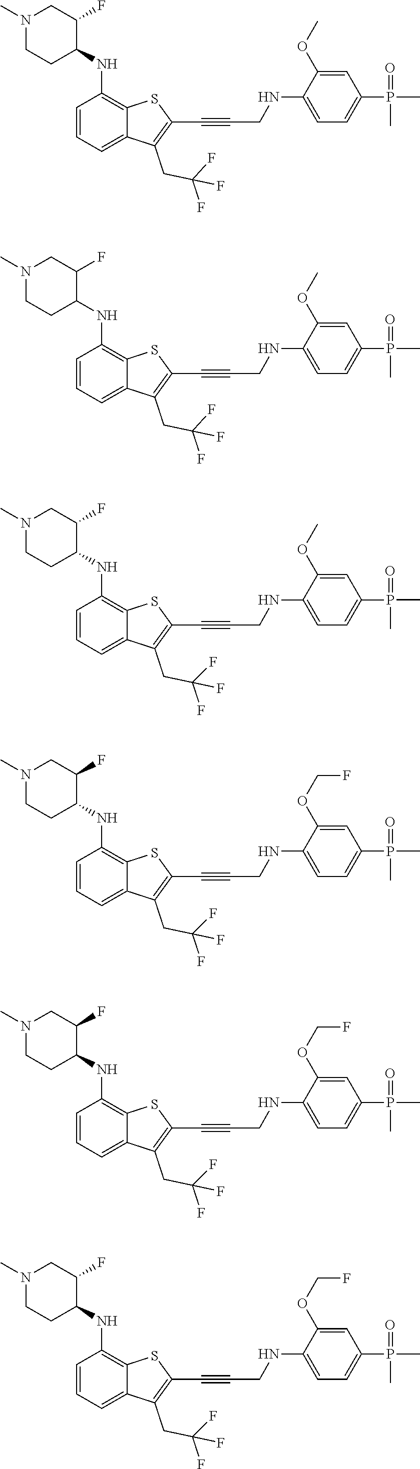

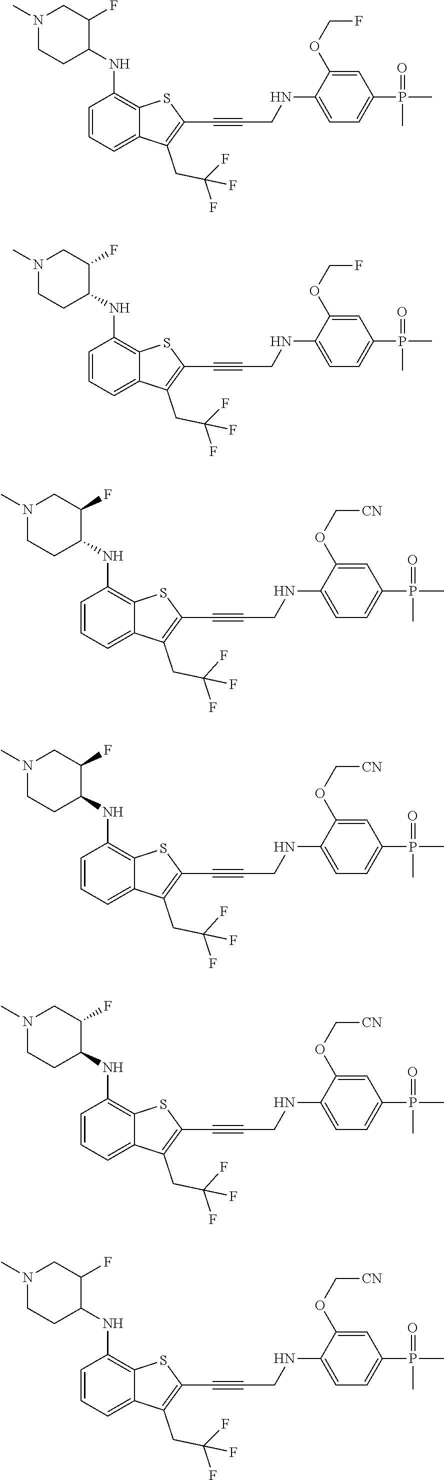

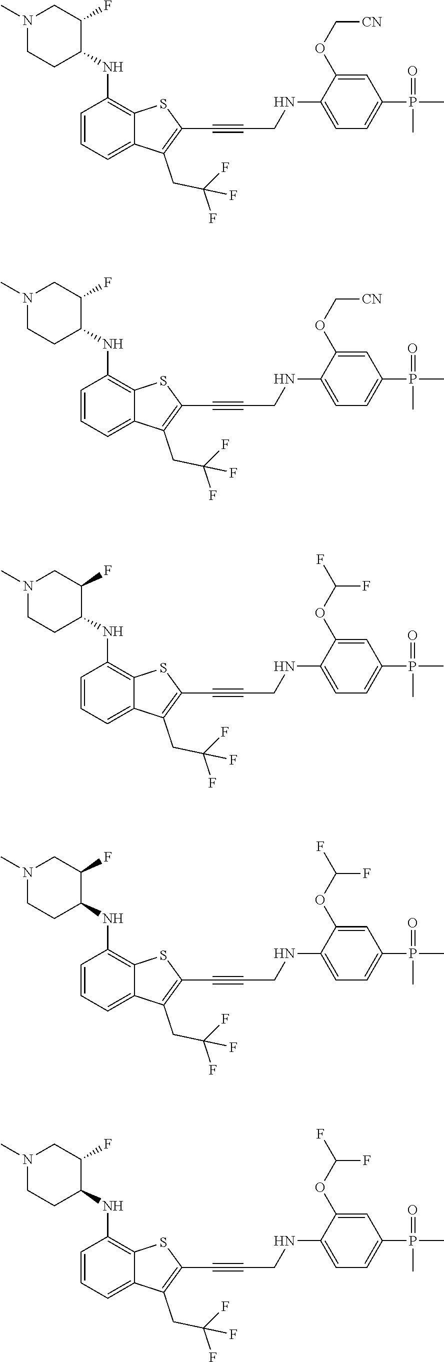

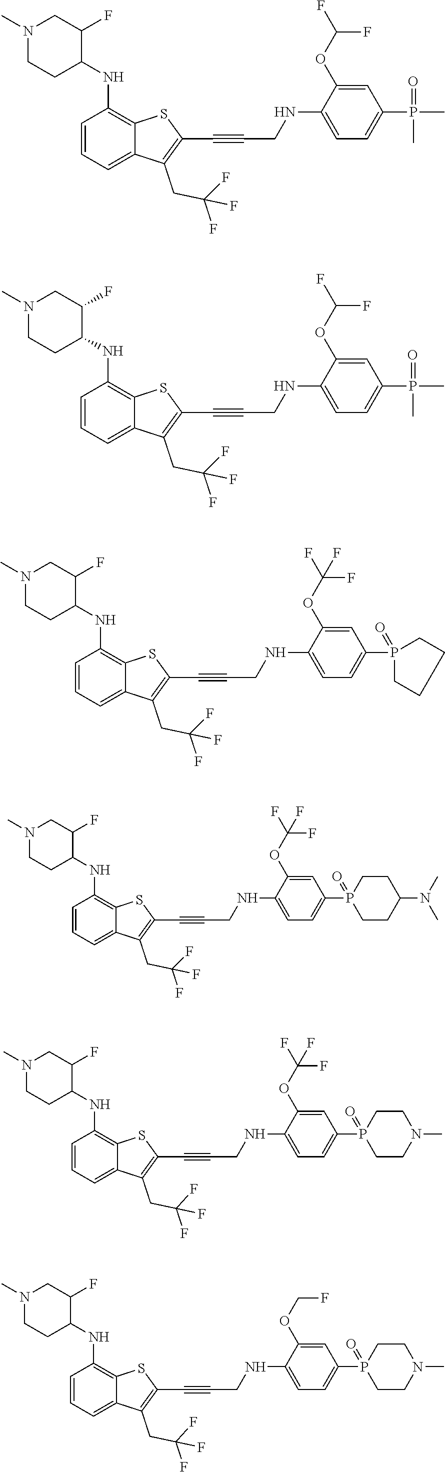

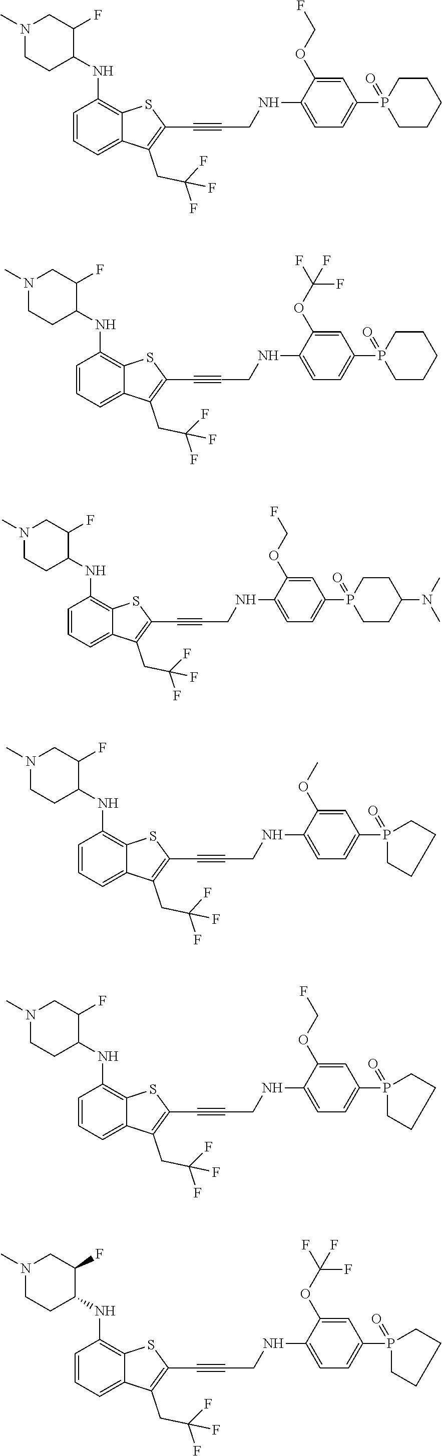

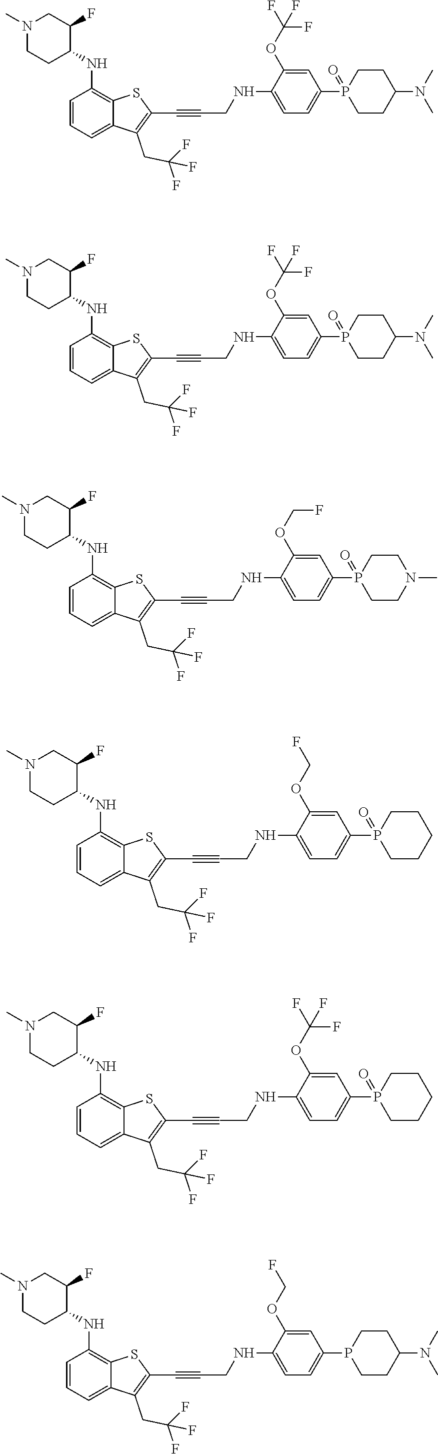

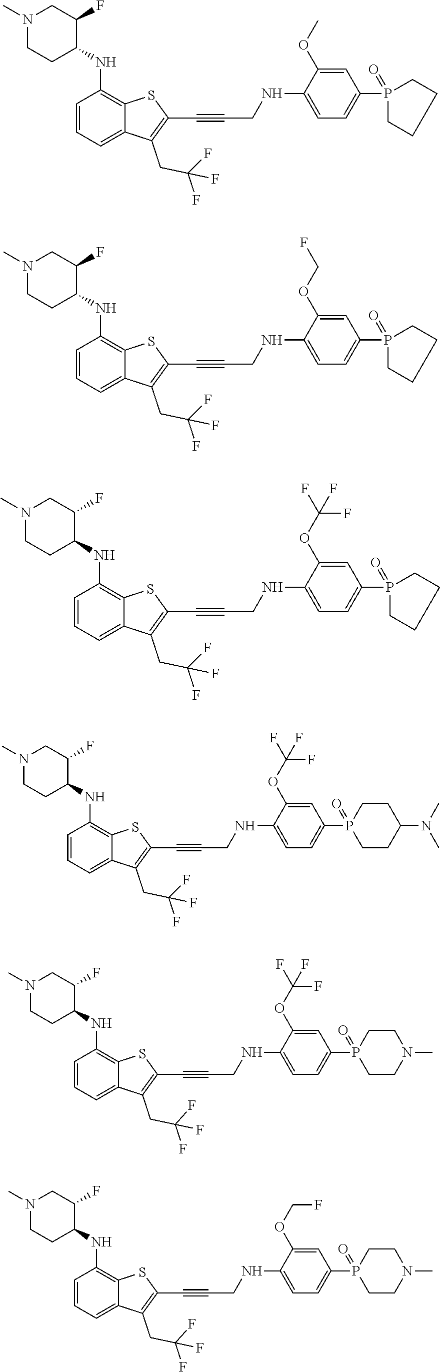

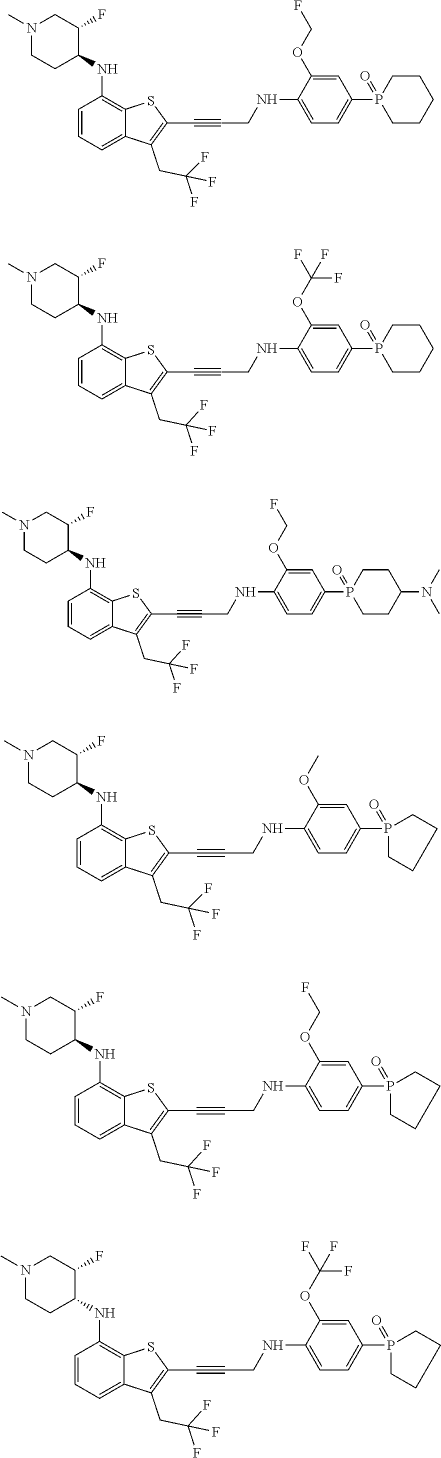

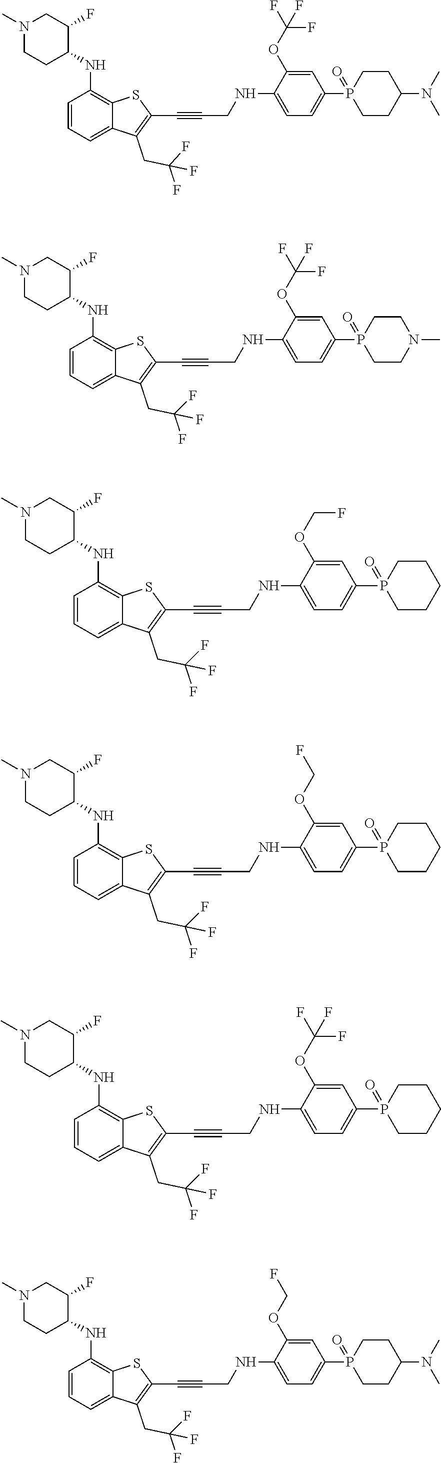

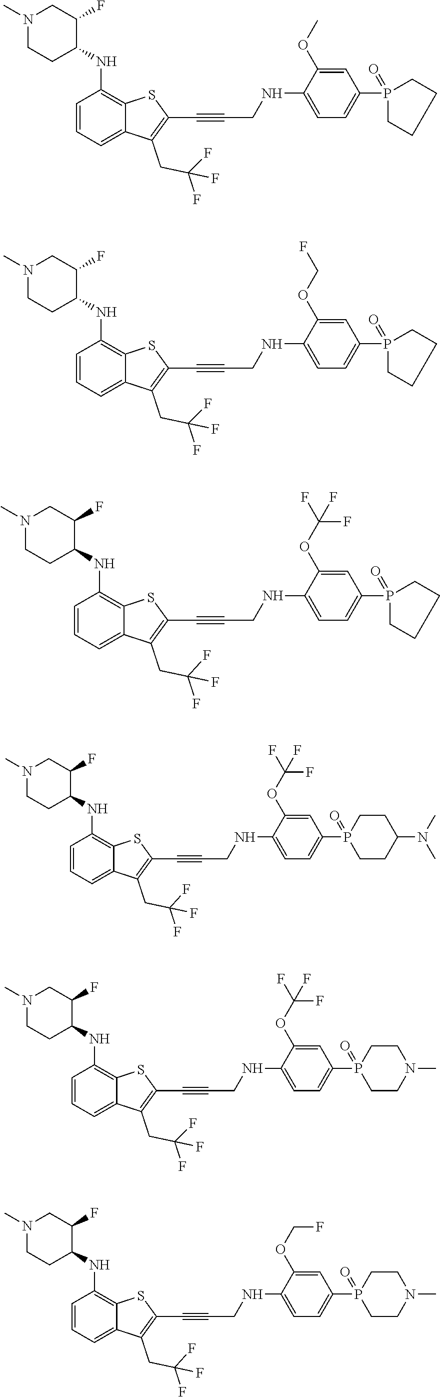

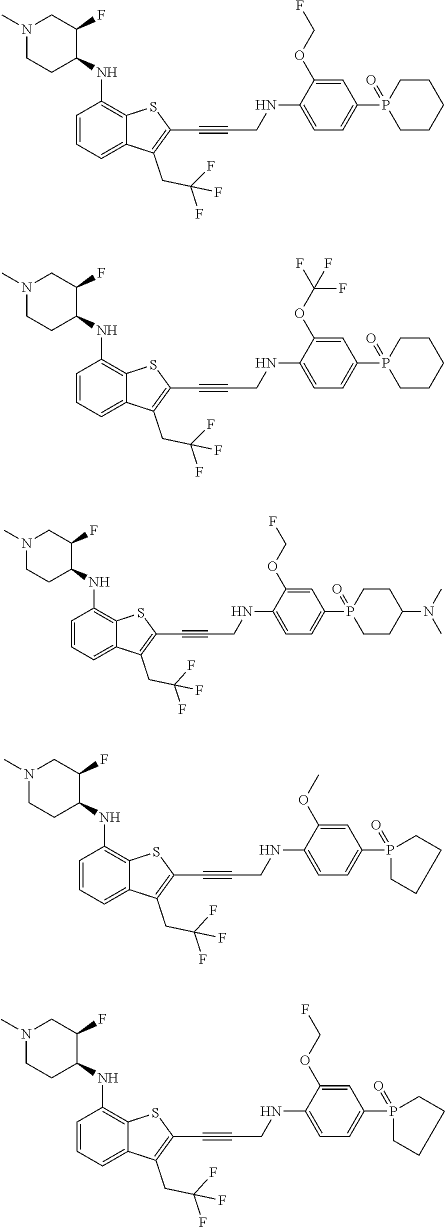

[59]. The compound according to any one of [1] to [53], wherein the compound of formula (I) is selected from:

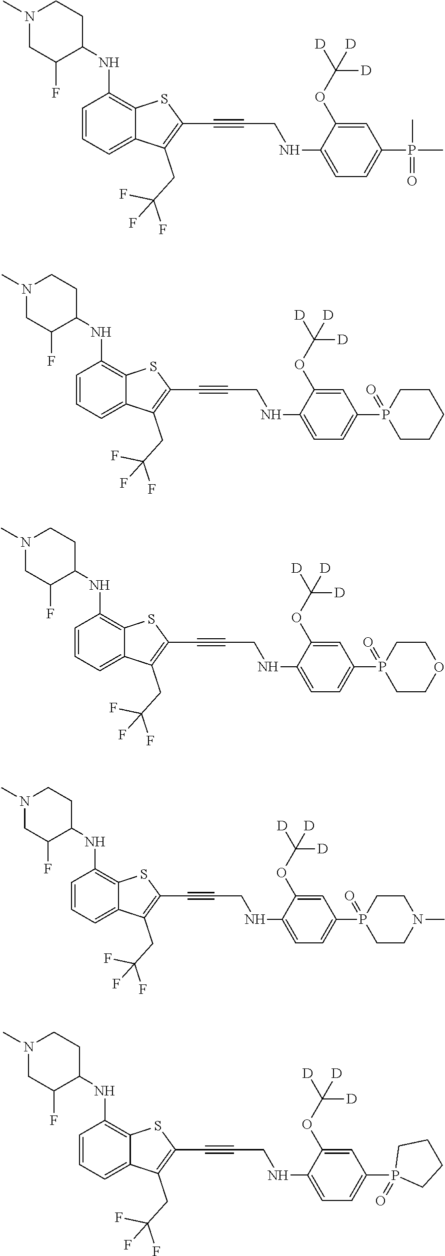

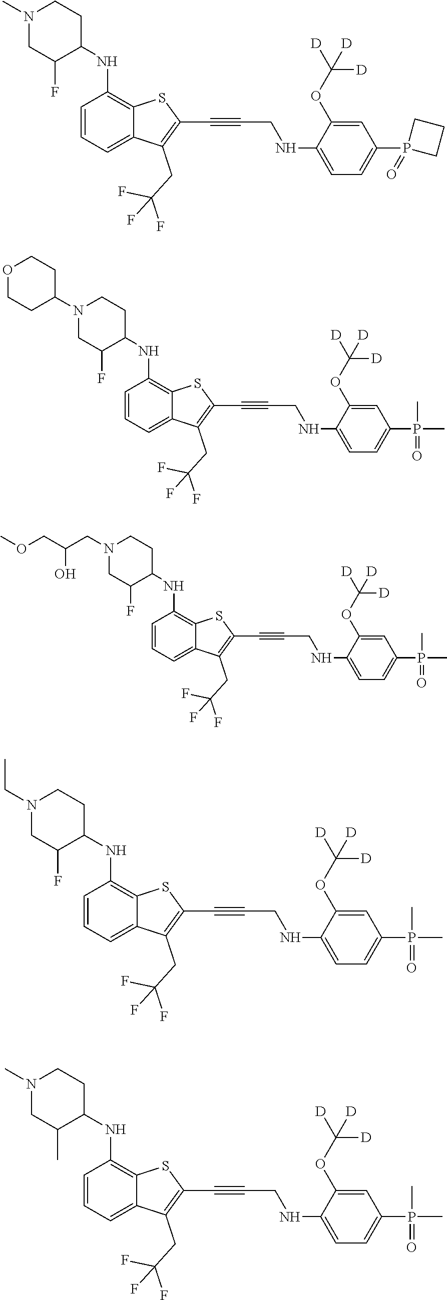

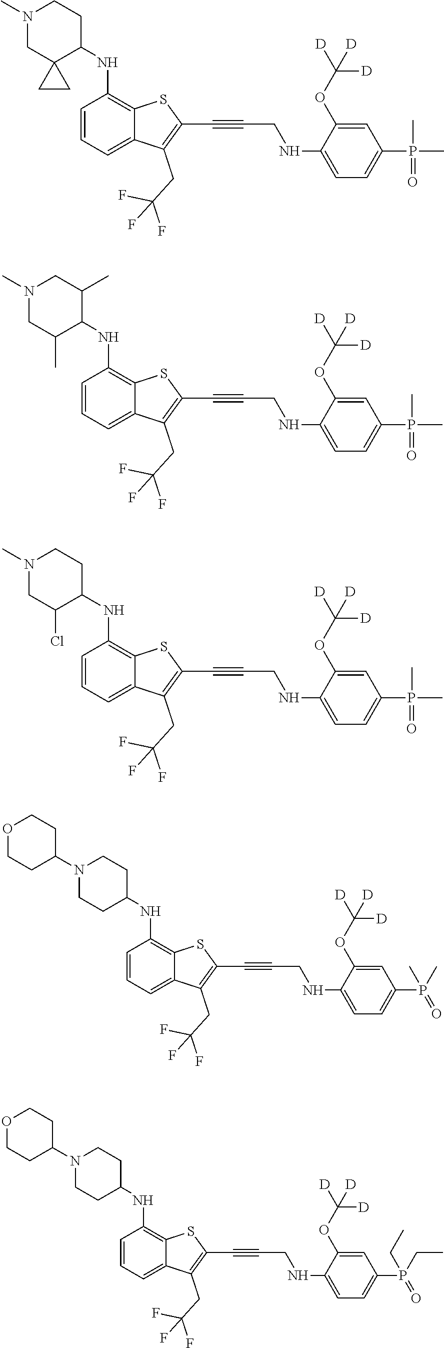

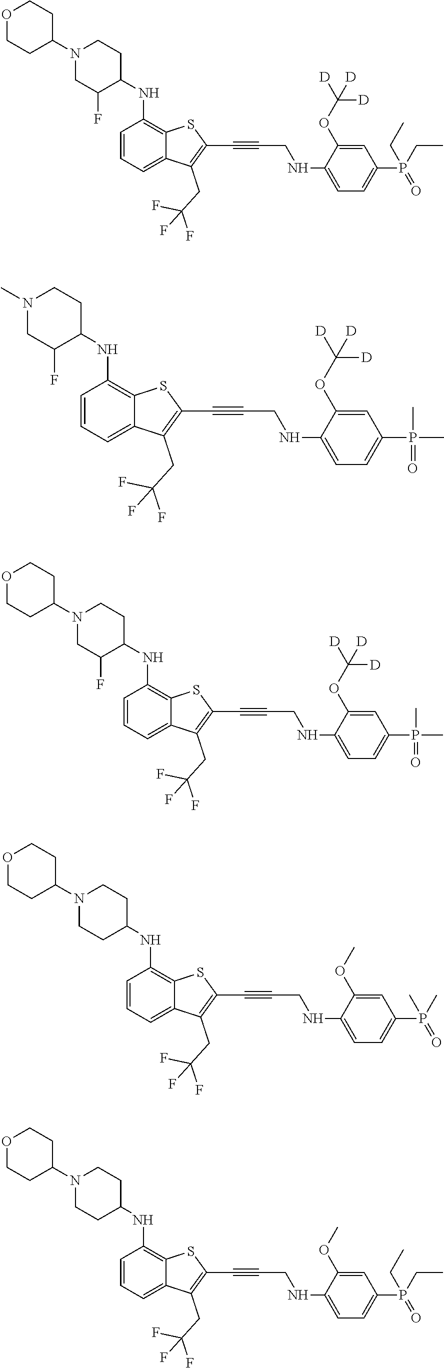

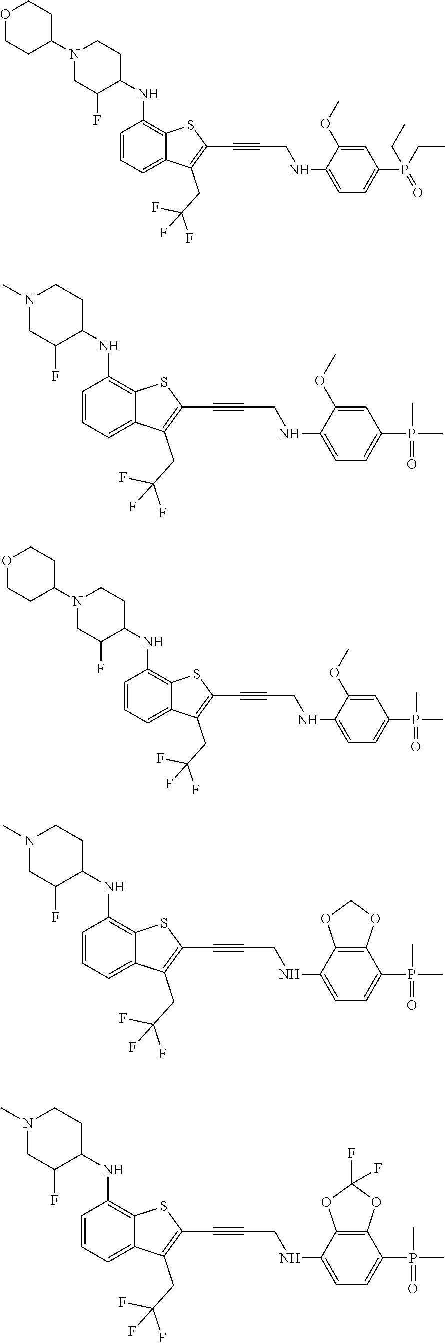

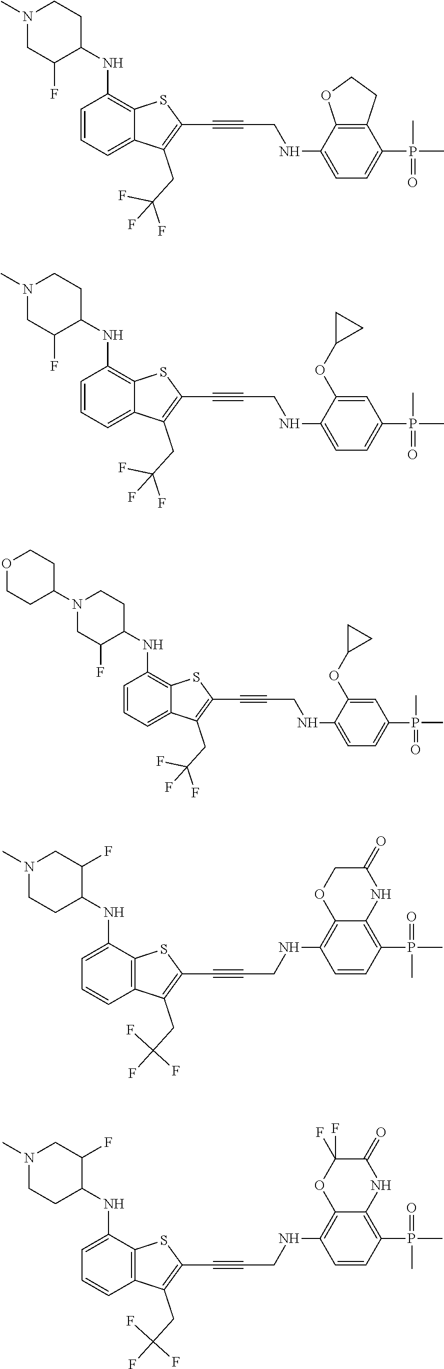

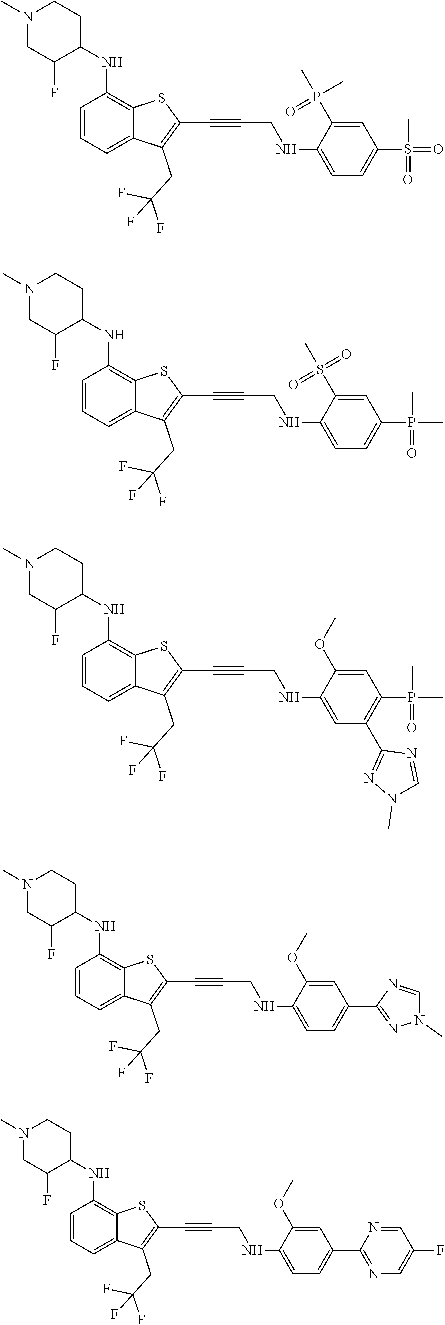

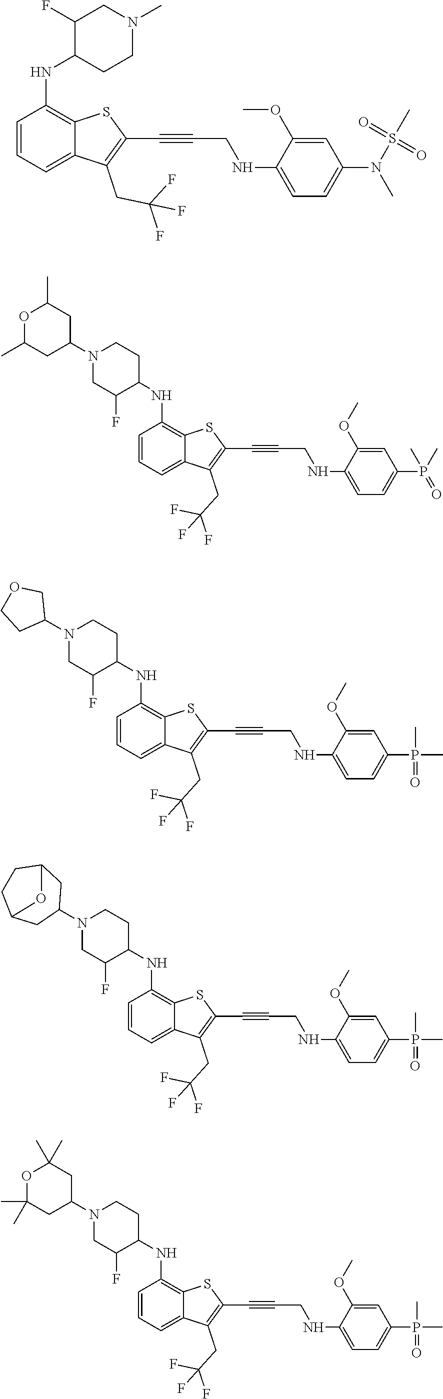

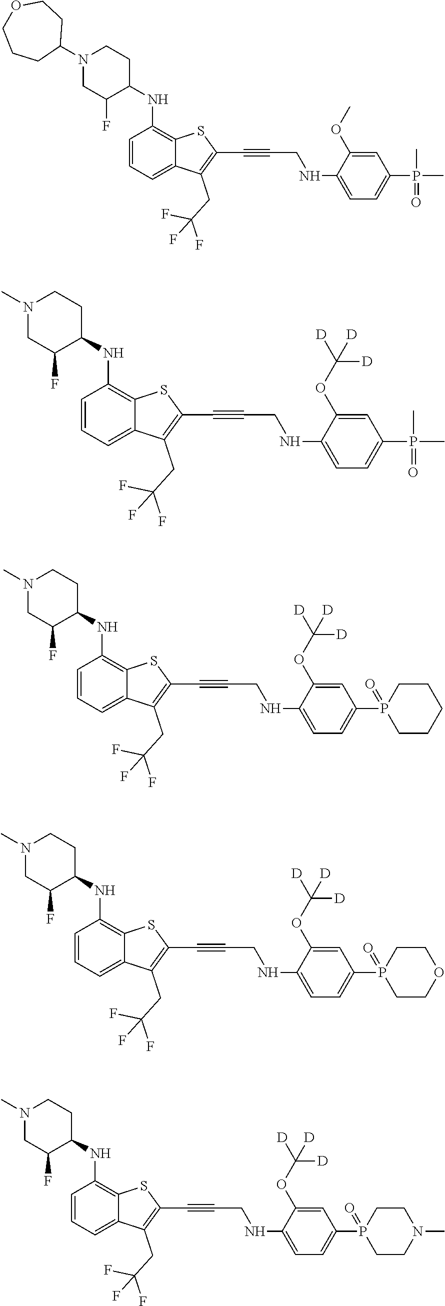

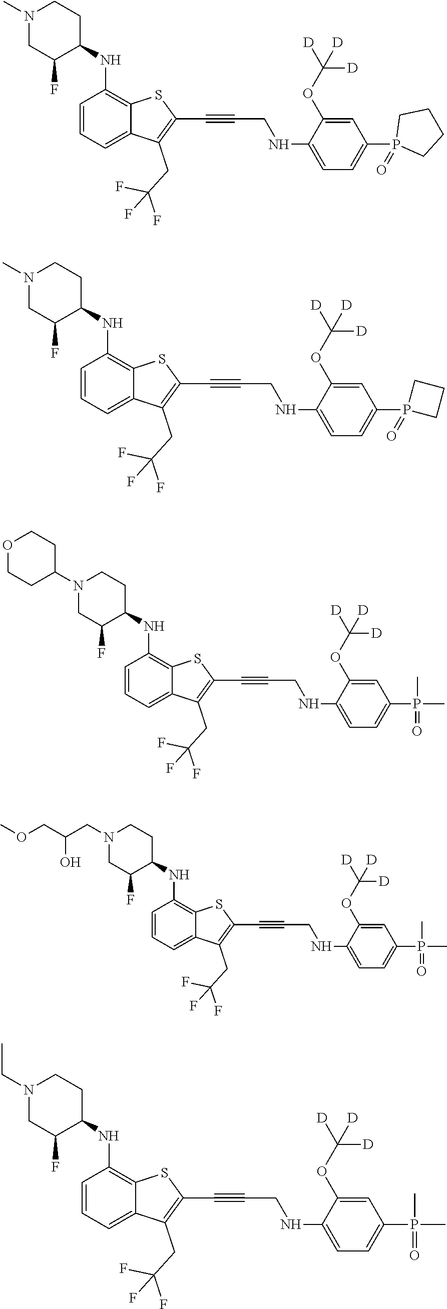

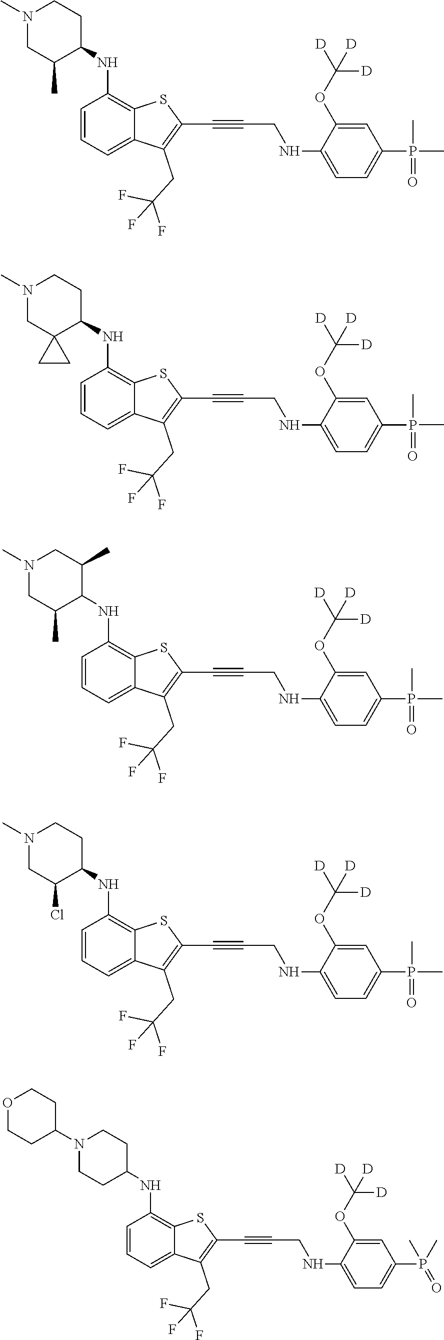

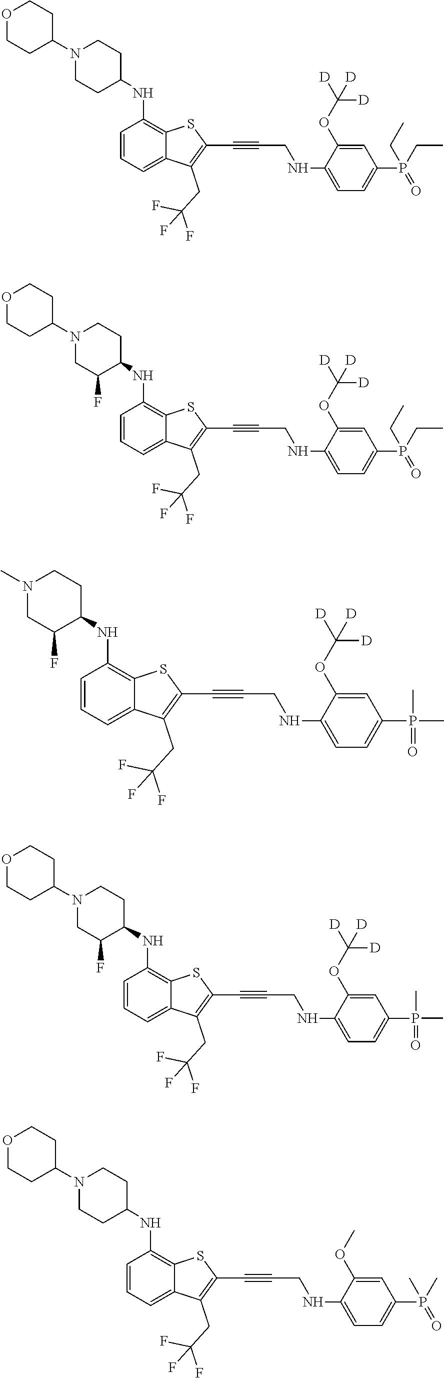

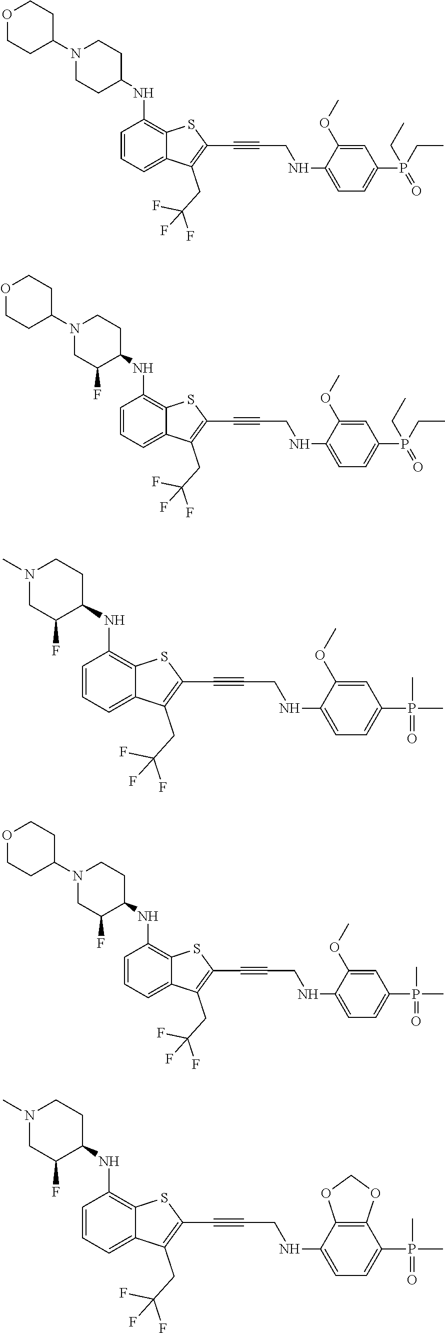

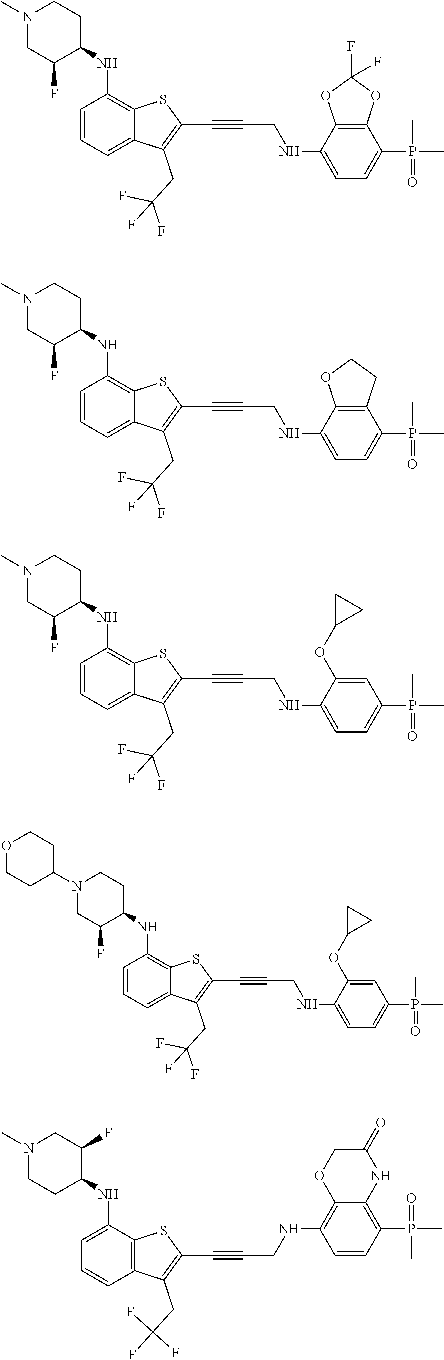

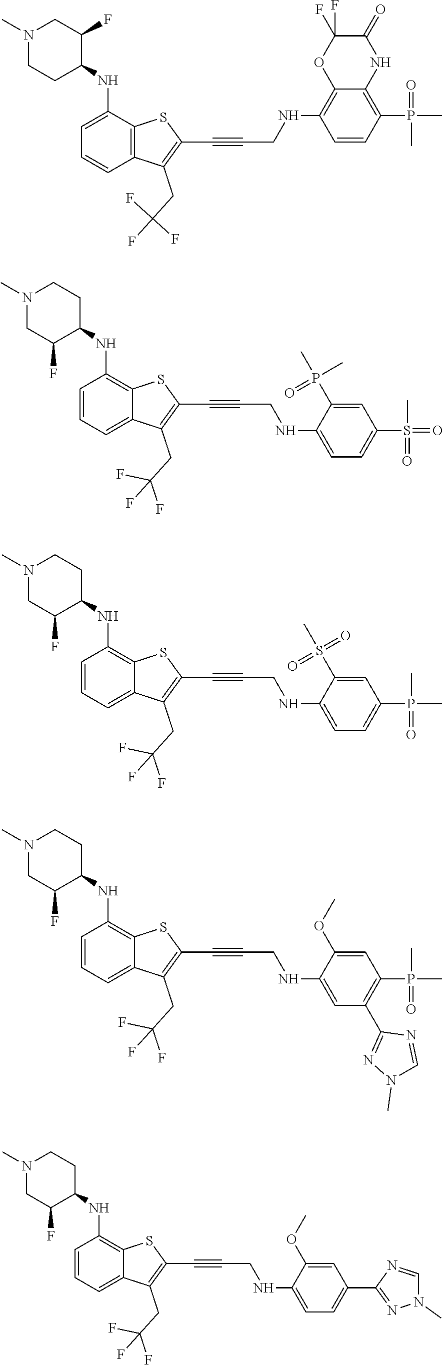

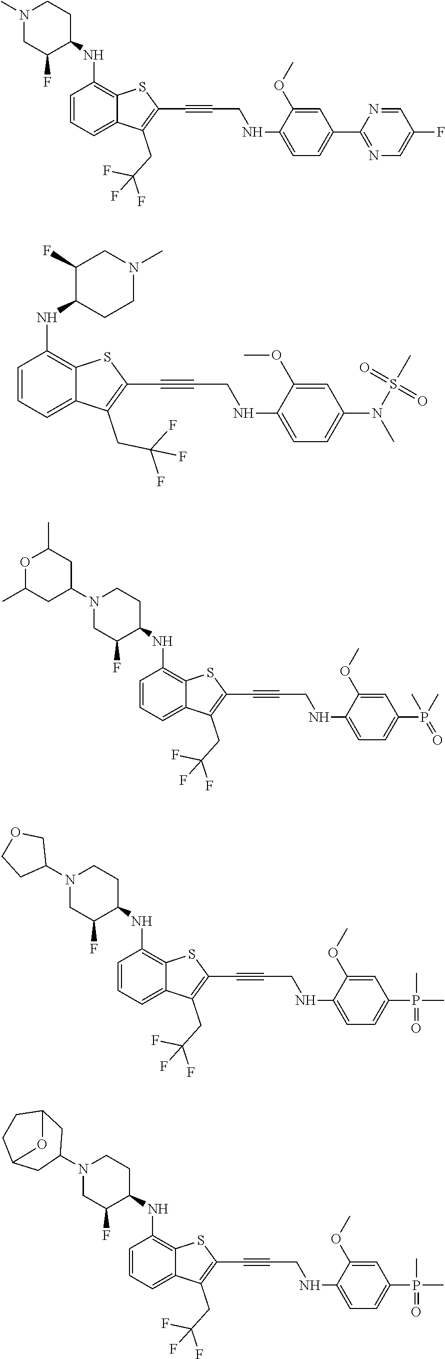 [SMILES: COc1cc(-c2ncc(F)cn2)ccc1NCC#Cc1sc2c(N[C@@H]3CCN(C)C[C@@H]3F)cccc2c1CC(F)(F)F ; COc1cc(N(C)S(C)(=O)=O)ccc1NCC#Cc1sc2c(N[C@@H]3CCN(C)C[C@@H]3F)cccc2c1CC(F)(F)F ; COc1cc(P(C)(C)=O)ccc1NCC#Cc1sc2c(N[C@@H]3CCN(C4CC(C)OC(C)C4)C[C@@H]3F)cccc2c1CC(F)(F)F ; COc1cc(P(C)(C)=O)ccc1NCC#Cc1sc2c(N[C@@H]3CCN(C4CCOC4)C[C@@H]3F)cccc2c1CC(F)(F)F ; COc1cc(P(C)(C)=O)ccc1NCC#Cc1sc2c(N[C@@H]3CCN(C4CC5CCC(C4)O5)C[C@@H]3F)cccc2c1CC(F)(F)F]

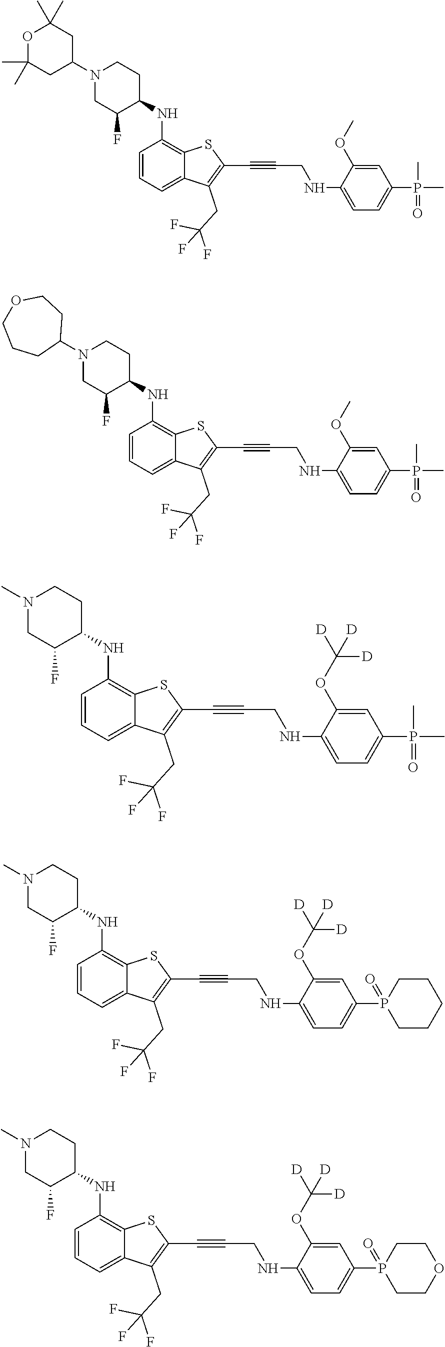

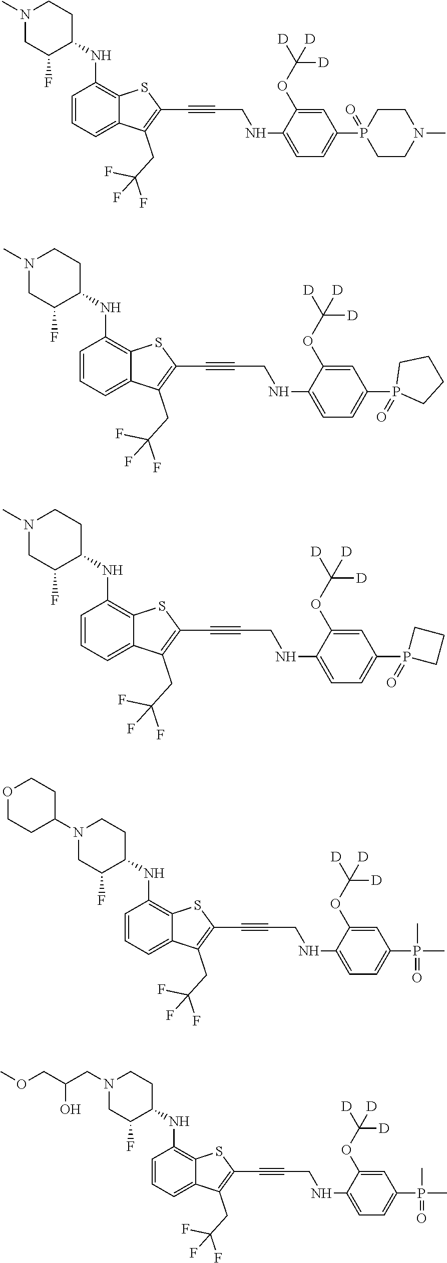

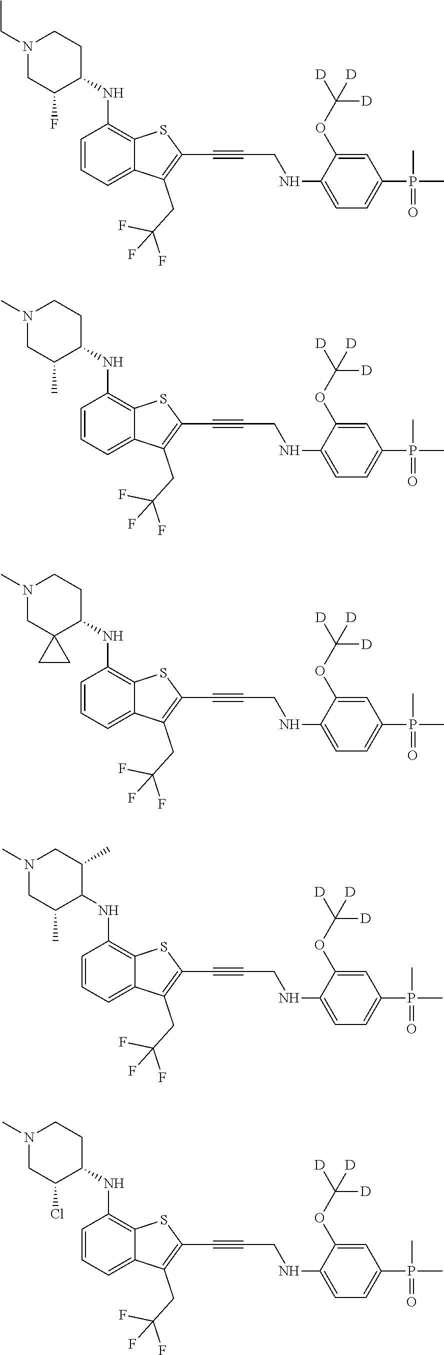

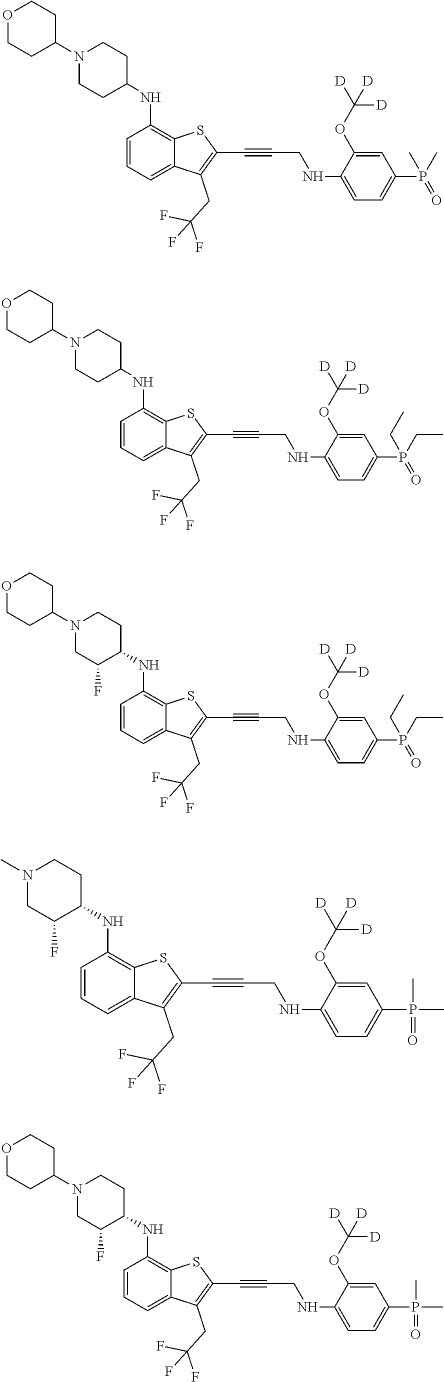

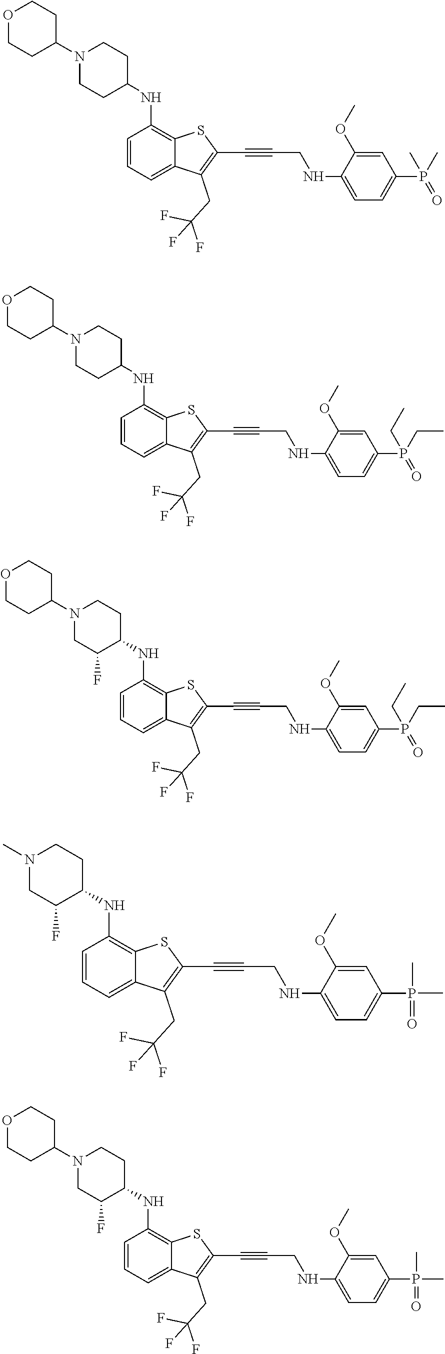

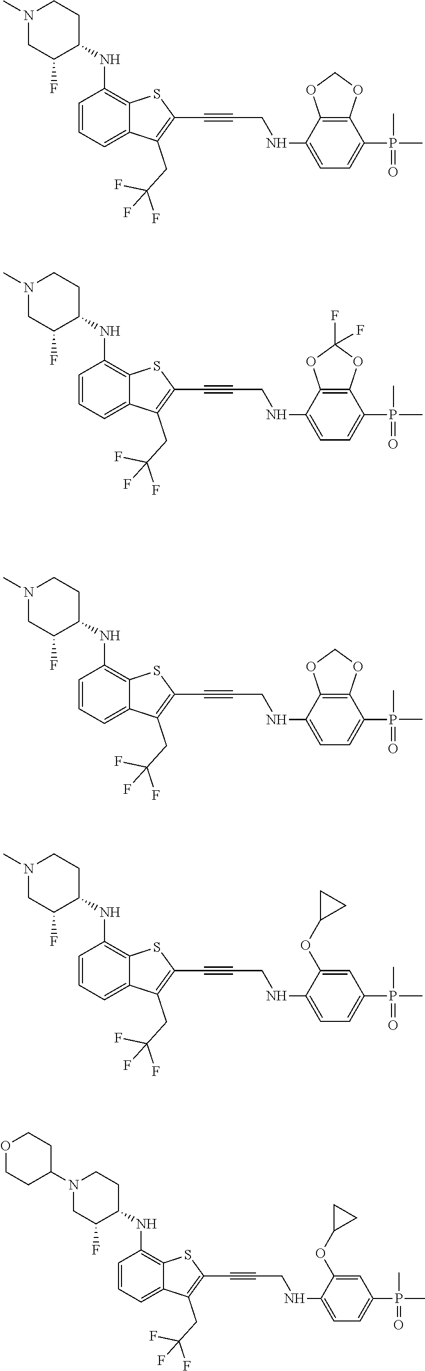

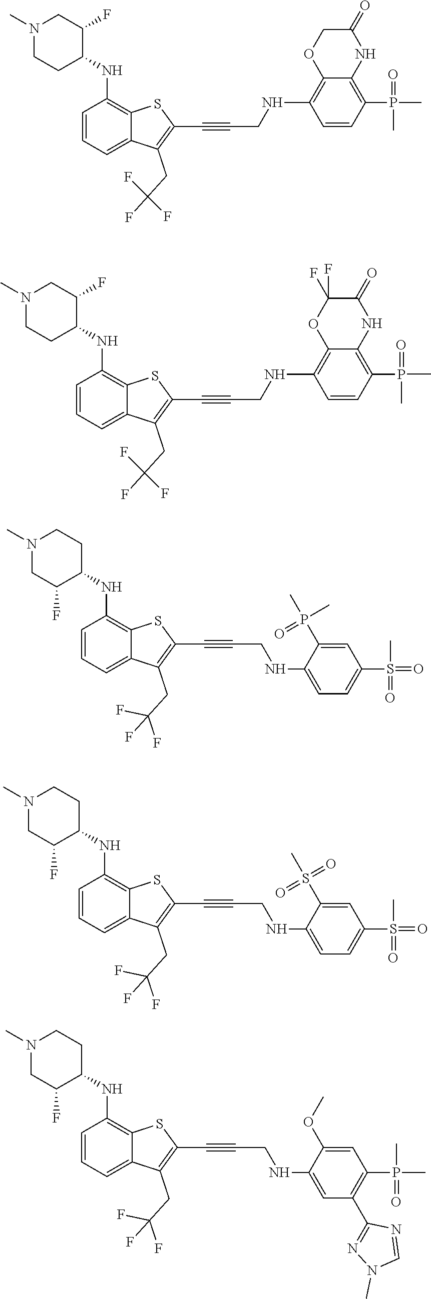

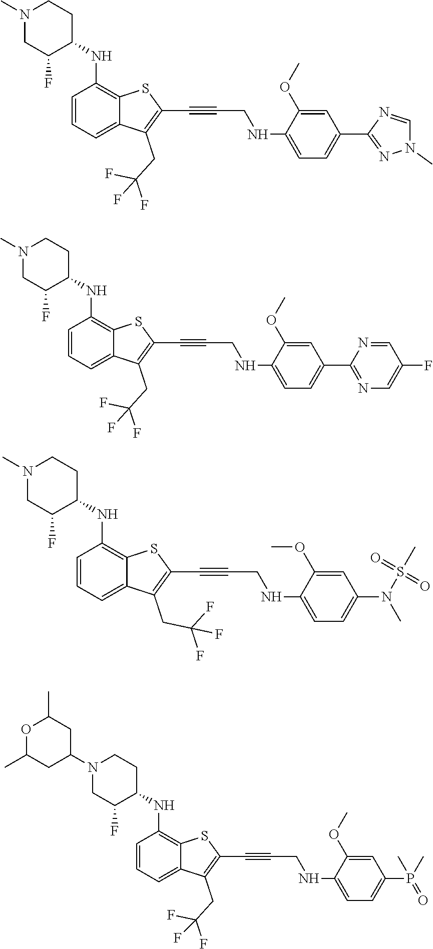

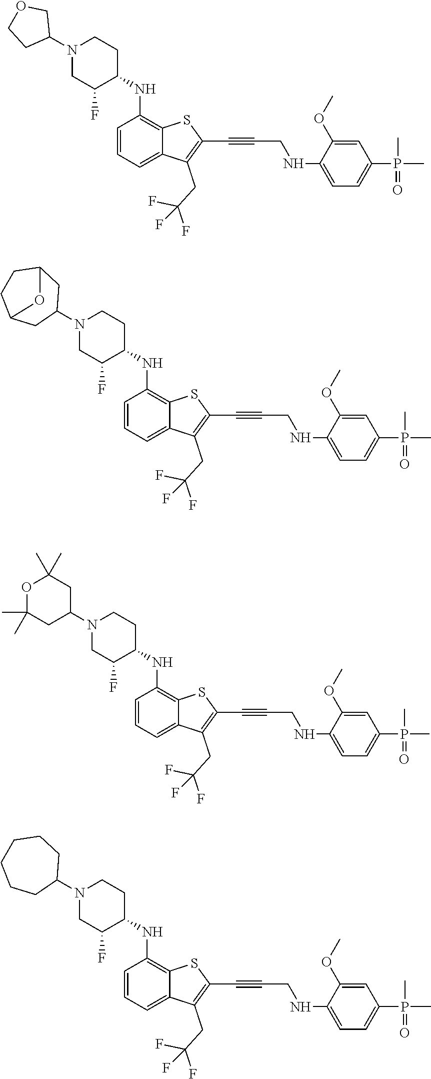

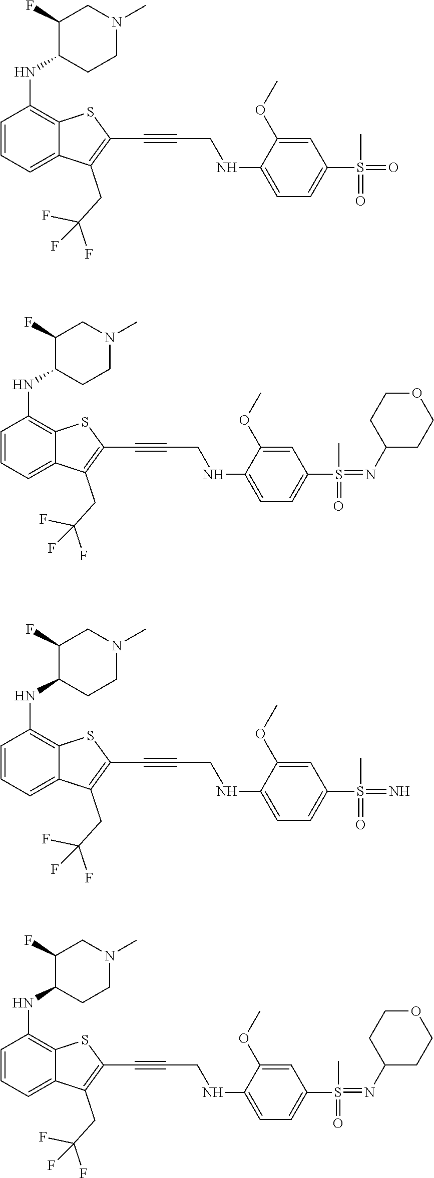 [SMILES: COc1cc(S(C)(=O)=O)ccc1NCC#Cc1sc2c(N[C@H]3CCN(C)C[C@@H]3F)cccc2c1CC(F)(F)F ; COc1cc(S(C)(=O)=NC2CCOCC2)ccc1NCC#Cc1sc2c(N[C@H]3CCN(C)C[C@@H]3F)cccc2c1CC(F)(F)F ; COc1cc(S(C)(=N)=O)ccc1NCC#Cc1sc2c(N[C@@H]3CCN(C)C[C@@H]3F)cccc2c1CC(F)(F)F ; COc1cc(S(C)(=O)=NC2CCOCC2)ccc1NCC#Cc1sc2c(N[C@@H]3CCN(C)C[C@@H]3F)cccc2c1CC(F)(F)F]

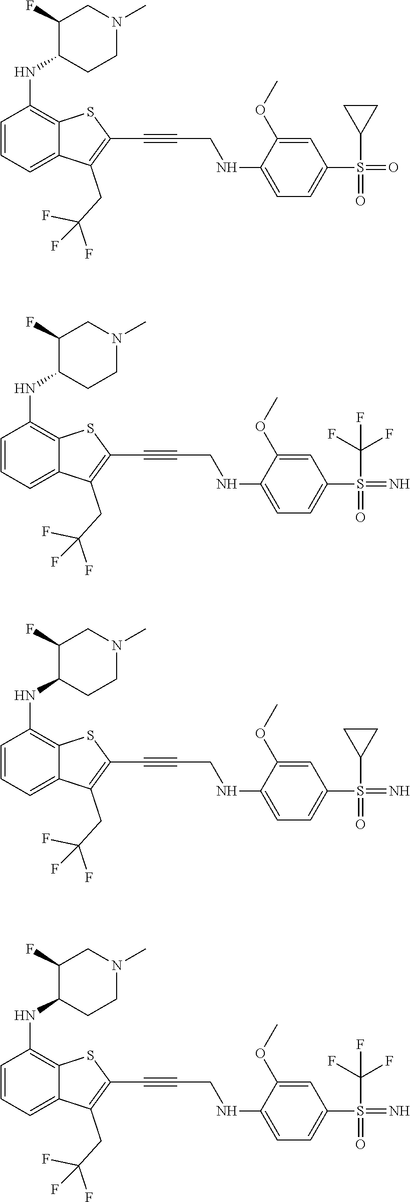 [SMILES: COc1cc(S(=O)(=O)C2CC2)ccc1NCC#Cc1sc2c(N[C@H]3CCN(C)C[C@@H]3F)cccc2c1CC(F)(F)F ; COc1cc(S(=N)(=O)C(F)(F)F)ccc1NCC#Cc1sc2c(N[C@H]3CCN(C)C[C@@H]3F)cccc2c1CC(F)(F)F ; COc1cc(S(=N)(=O)C2CC2)ccc1NCC#Cc1sc2c(N[C@@H]3CCN(C)C[C@@H]3F)cccc2c1CC(F)(F)F ; COc1cc(S(=N)(=O)C(F)(F)F)ccc1NCC#Cc1sc2c(N[C@@H]3CCN(C)C[C@@H]3F)cccc2c1CC(F)(F)F]

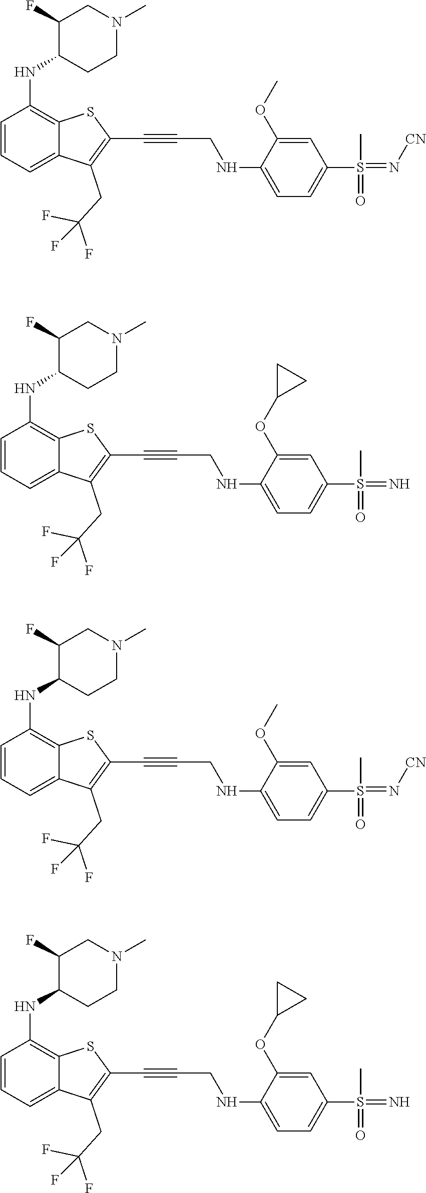 [SMILES: COc1cc(S(C)(=O)=NC#N)ccc1NCC#Cc1sc2c(N[C@H]3CCN(C)C[C@@H]3F)cccc2c1CC(F)(F)F ; CN1CC[C@H](Nc2cccc3c(CC(F)(F)F)c(C#CCNc4ccc(S(C)(=N)=O)cc4OC4CC4)sc23)[C@@H](F)C1 ; COc1cc(S(C)(=O)=NC#N)ccc1NCC#Cc1sc2c(N[C@@H]3CCN(C)C[C@@H]3F)cccc2c1CC(F)(F)F ; CN1CC[C@@H](Nc2cccc3c(CC(F)(F)F)c(C#CCNc4ccc(S(C)(=N)=O)cc4OC4CC4)sc23)[C@@H](F)C1]

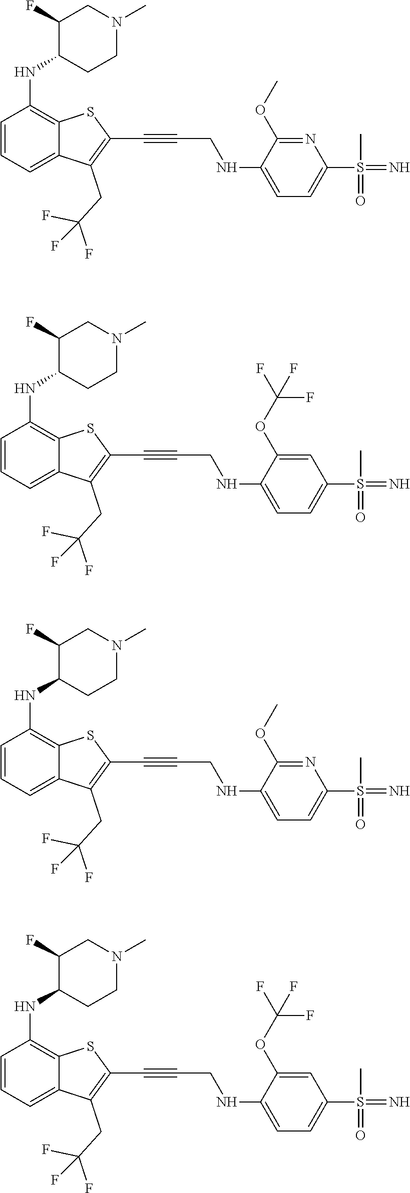 [SMILES: COc1nc(S(C)(=N)=O)ccc1NCC#Cc1sc2c(N[C@H]3CCN(C)C[C@@H]3F)cccc2c1CC(F)(F)F ; CN1CC[C@H](Nc2cccc3c(CC(F)(F)F)c(C#CCNc4ccc(S(C)(=N)=O)cc4OC(F)(F)F)sc23)[C@@H](F)C1 ; COc1nc(S(C)(=N)=O)ccc1NCC#Cc1sc2c(N[C@@H]3CCN(C)C[C@@H]3F)cccc2c1CC(F)(F)F ; CN1CC[C@@H](Nc2cccc3c(CC(F)(F)F)c(C#CCNc4ccc(S(C)(=N)=O)cc4OC(F)(F)F)sc23)[C@@H](F)C1]

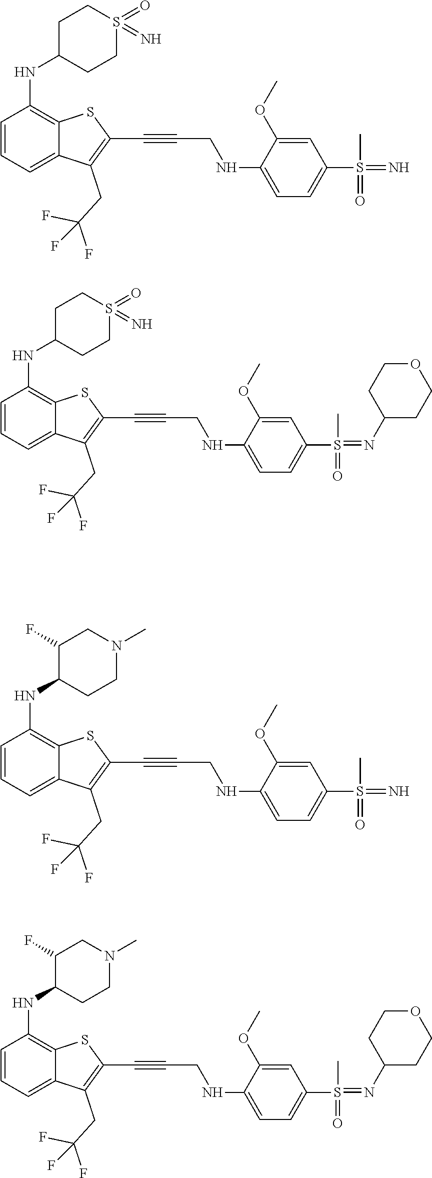

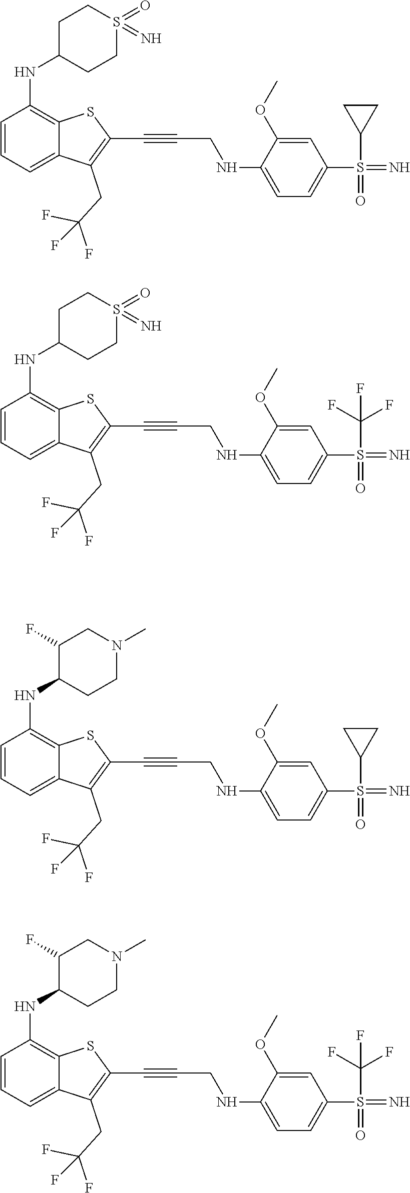

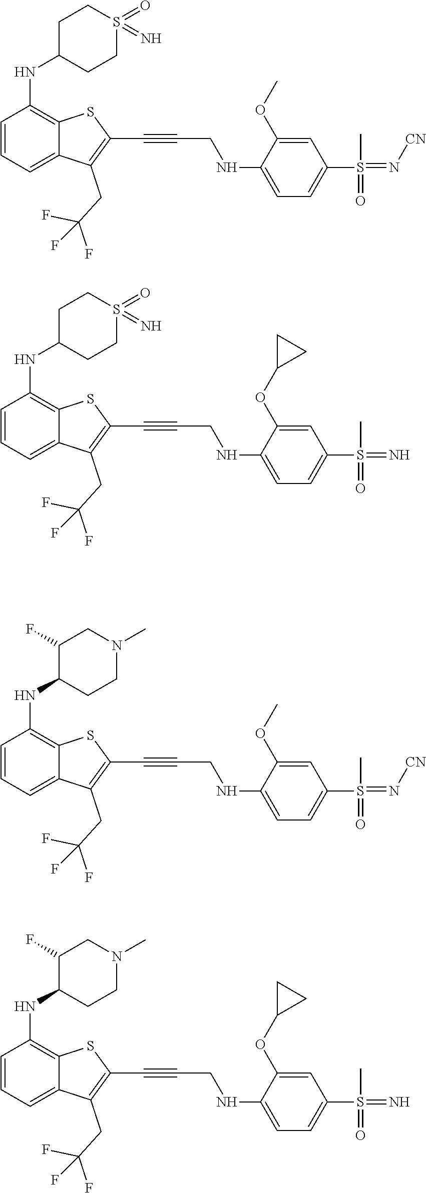

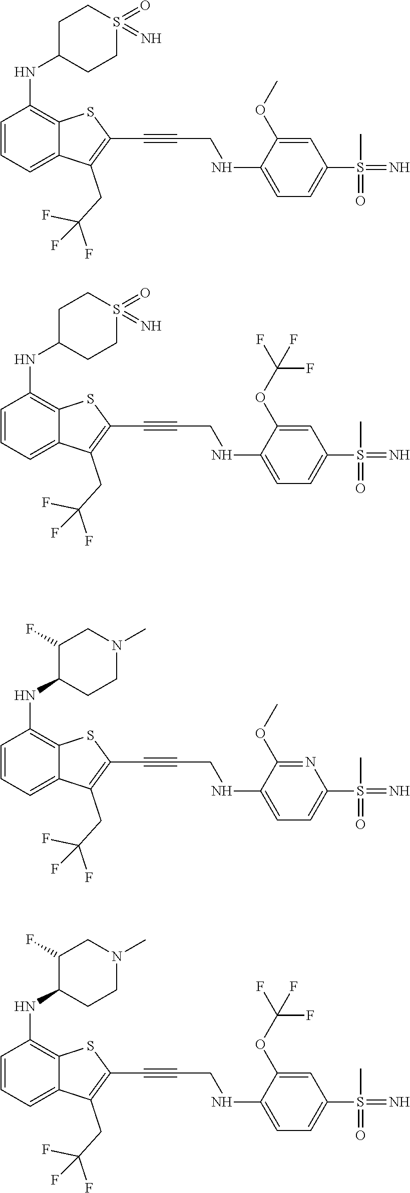

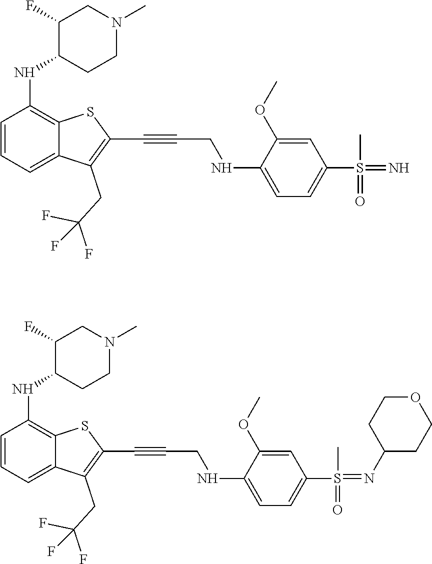 [SMILES: COc1cc(S(C)(=N)=O)ccc1NCC#Cc1sc2c(N[C@H]3CCN(C)C[C@H]3F)cccc2c1CC(F)(F)F ; COc1cc(S(C)(=O)=NC2CCOCC2)ccc1NCC#Cc1sc2c(N[C@H]3CCN(C)C[C@H]3F)cccc2c1CC(F)(F)F]

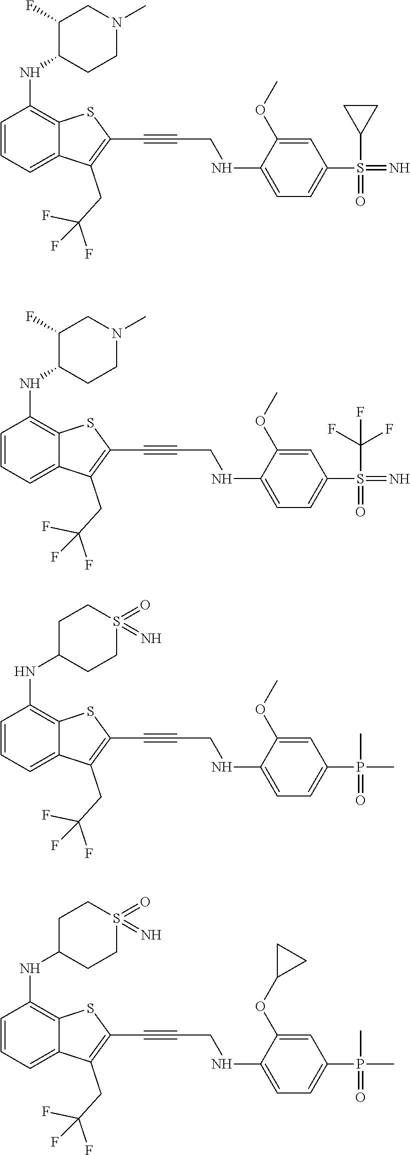

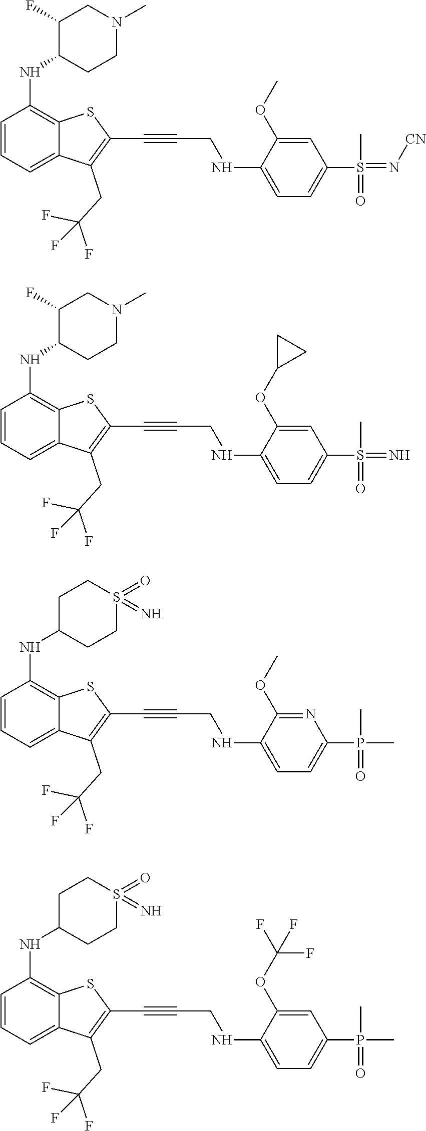

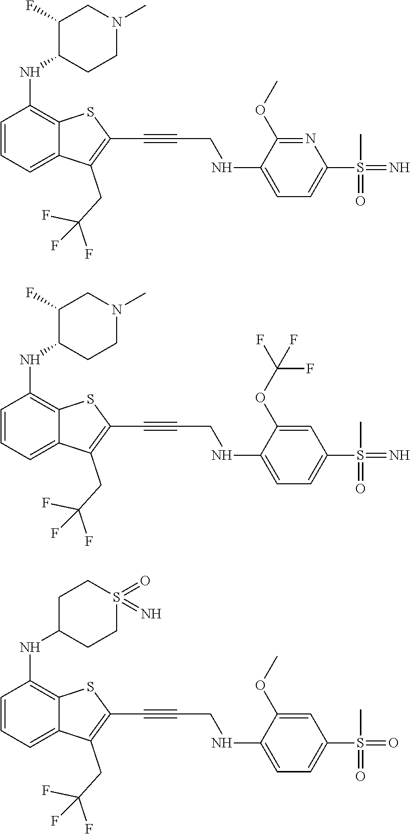

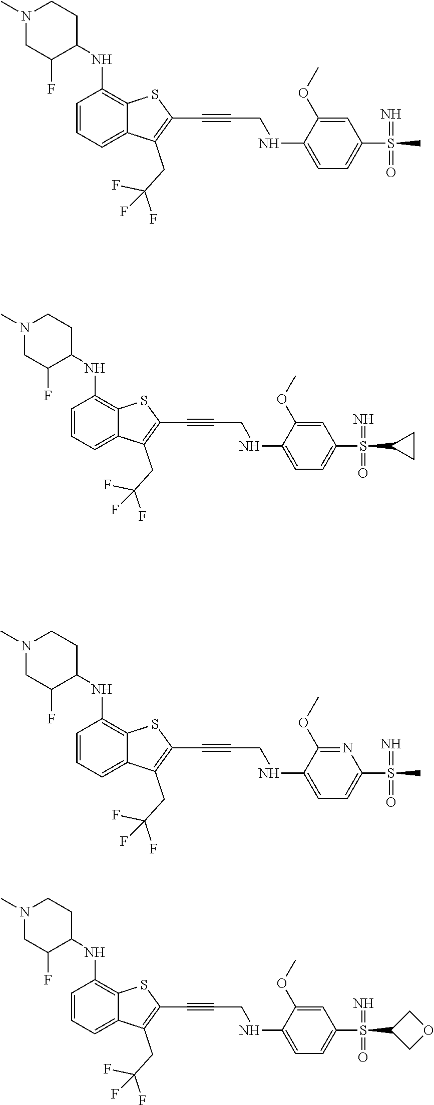

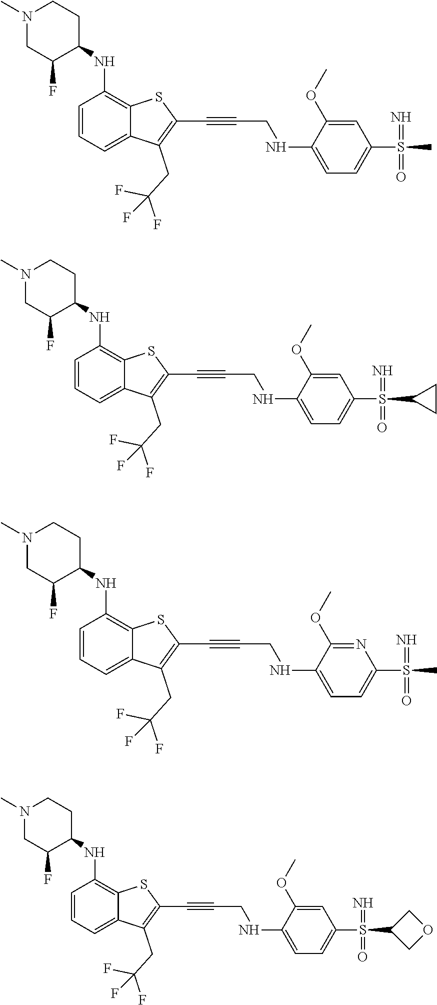

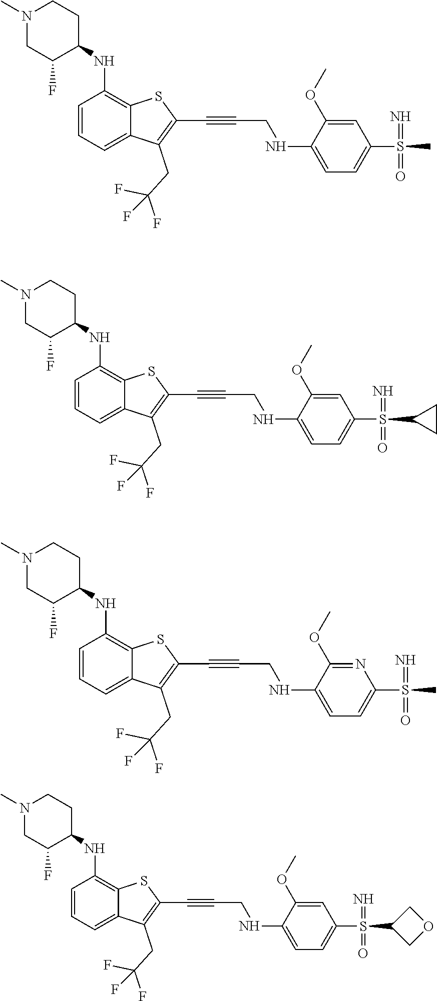

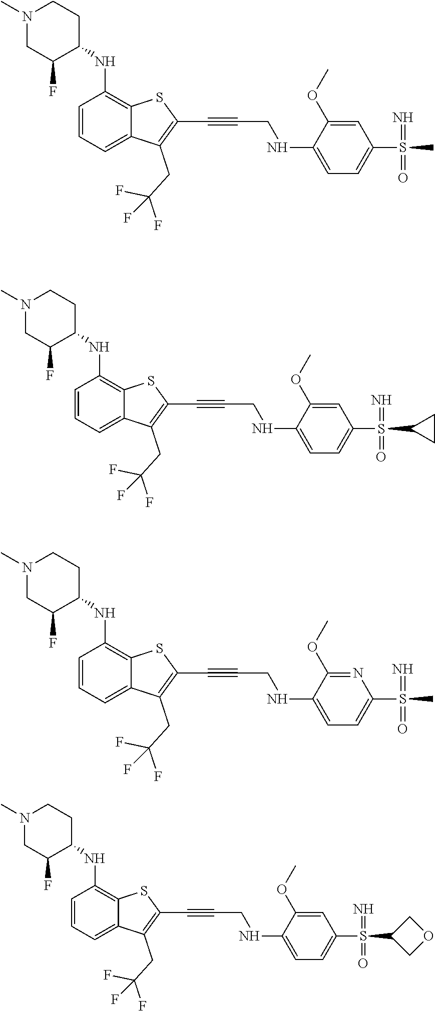

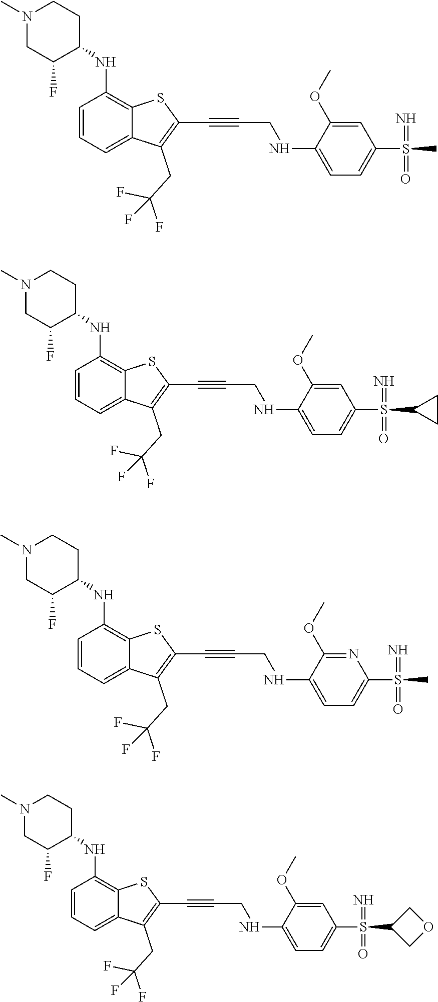

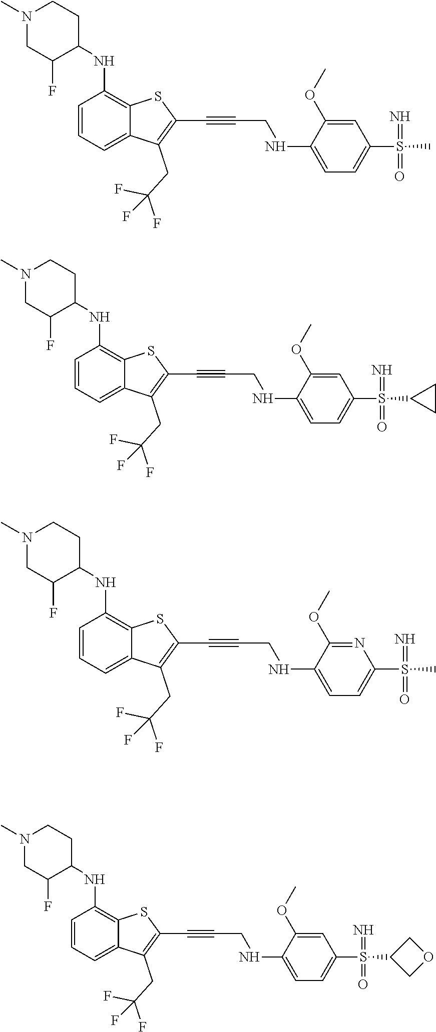

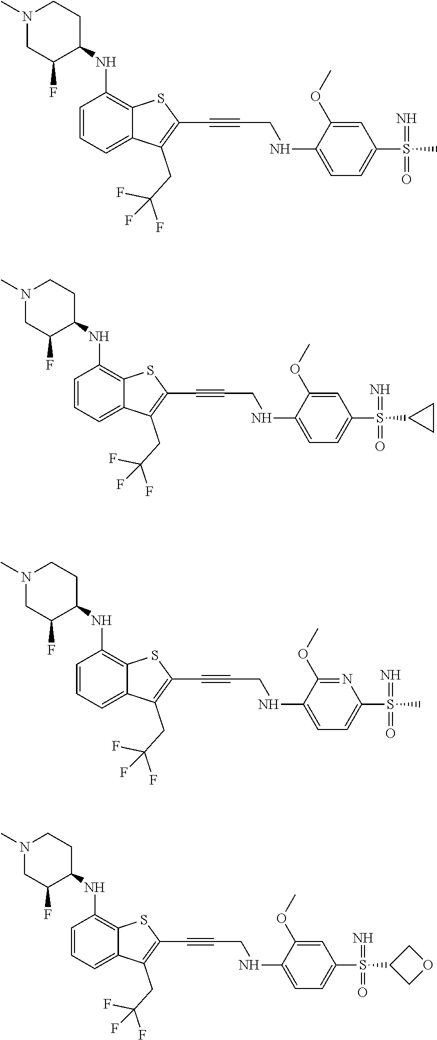

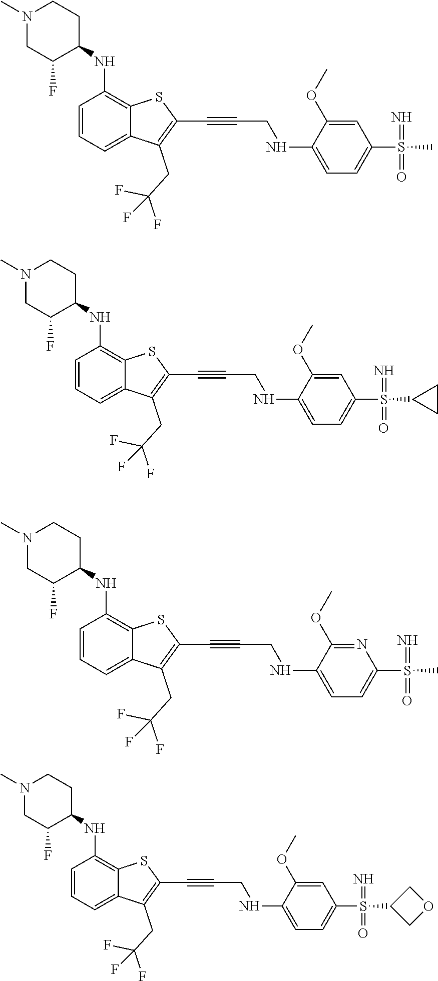

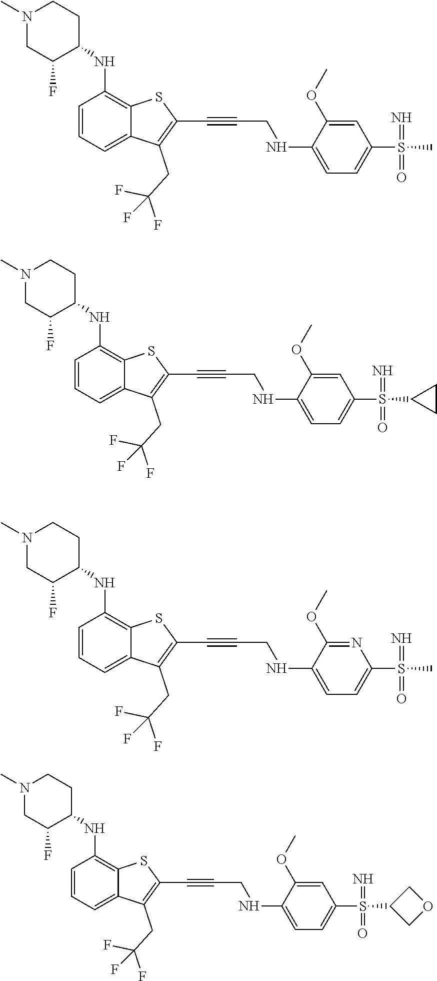

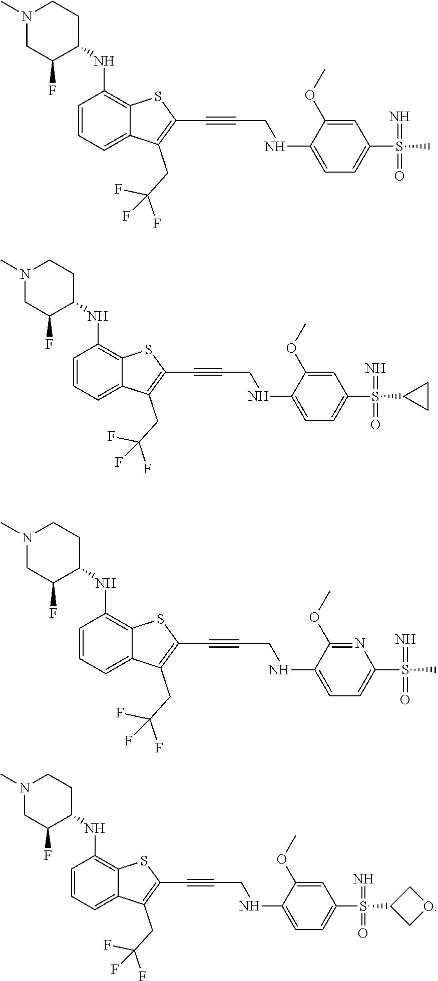

[60]. A compound of formula (IN-1):

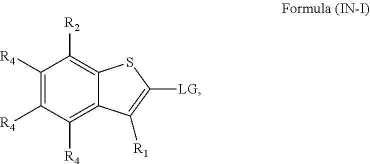

wherein R₁, R₂ or R₄ is defined in the same manner as that in any one of [1]-[59], and LG is selected from a leaving group, such as halogen, preferably bromine or iodine.

[61]. A method for preparation of the compound of formula (I-1) according to any one of [1]-[59], comprising steps of:

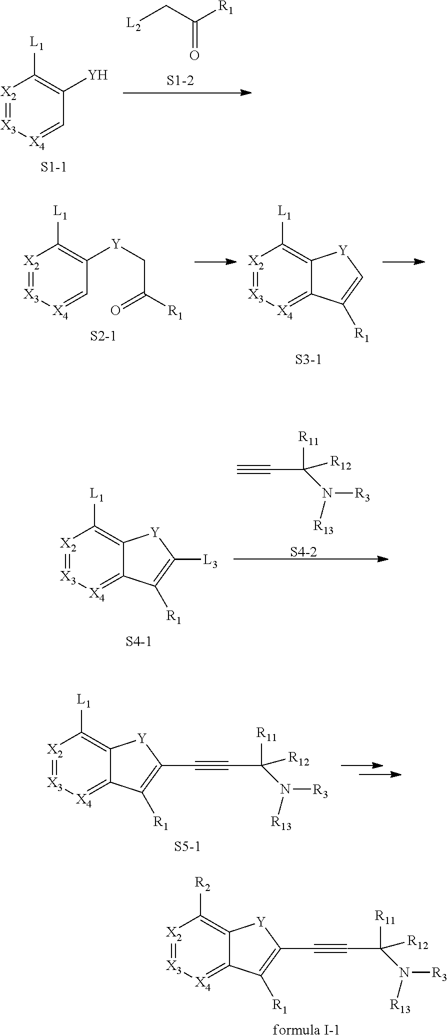

(1) reacting a compound S1-1 as starting material with a compound S1-2 in the presence of an alkaline agent to form a compound of S2-1;
(2) converting the compound of S2-1 into a compound S3-1 under acidic condition;
(3) converting the compound S3-1 in the presence of halogenating reagent into a compound S4-1; (4) reacting the compound S4-1 with reagent S4-2 in the presence of a coupling catalyst to form a compound S5-1;
(5) converting the compound S5-1 into the compound of formula (I-1) through one or more steps of reaction such as a reductive amination reaction, a deprotecting reaction or a combination thereof;
wherein L₁, L₂ and L₃ independently represent a leaving group;
Y is selected from O or S;
X₂, X₃, X₄, R₁, R₂, R₁₁, R₁₂, R₁₃, R₃, R₅₁, R₅₂ and m are each defined as in any one of [1]-[59].

[62]. The method according to [61], wherein the alkaline agent is K₂CO₃.

[63]. The method according to [61] or [62], wherein the acidic condition is polyphosphoric acid.

[64]. The method according to [61] or [62], wherein the halogenating reagent is NIS.

[65]. The method according to [61] or [62], wherein group L₁ of the compound S5-1 is converted to —NH₂, and then further converted to the compound of formula (I-1) through a reductive amination reaction, a deprotecting reaction or a combination thereof, when R₂ is —NR₅₁R₅₂.

[66]. The method according to [61] or [62], wherein the coupling catalyst comprises Pd-containing coupling catalyst, such as Pd(PPh₃)₂Cl₂ or Pd(dppf)Cl₂.

[67]. The method according to [61] or [62], wherein the leaving group is halogen, such as —Cl, —Br or —I.

[68]. The method according to [61] or [62], wherein L₁ is selected from —Br; L₂ is selected from —Br; and L₃ is selected from —I.

[69]. A pharmaceutical composition, comprising a therapeutically effective amount of a compound of formula (I), or a stereoisomer, tautomer, deuterated derivative, prodrug or pharmaceutically acceptable salt thereof according to any one of [1]-[59], and a pharmaceutically acceptable carrier, diluent, or excipient.

[70]. Use of a compound of formula (I), or a stereoisomer, tautomer, deuterated derivative, prodrug or pharmaceutically acceptable salt thereof according to any one of [1]-[59], or a pharmaceutical composition according to [69] in the manufacture of a medicament for prevention or treatment of a disease or condition in a subject.

[71]. The use according to [70], wherein the disease or condition is cancer, preferably solid tumor, e.g. advanced solid tumor.

[72]. The use according to [71], wherein the cancer cell expresses the p53 mutant.

[73]. The use according to [72], wherein the p53 mutant has a mutation at amino acid Val143, His168, Arg175, Tyr220, Gly245, Arg248, Arg249, Phe270, Arg273, Arg282, and/or a combination thereof.

[74]. The use according to [72], wherein the p53 mutant is V157F, R175H, Y220C, G245S, R248Q, R248W, R249S, R273H, R273C, R282W, and/or a combination thereof, preferably Y220C.

[75]. The use according to any one of [70] to [74], wherein the disease or condition is selected from the group consisting of ovarian cancer, breast cancer, lung cancer and/or a combination thereof.

[76]. A compound of formula (I), or a stereoisomer, tautomer, deuterated derivative, prodrug or pharmaceutically acceptable salt thereof according to any one of [1]-[59], or a pharmaceutical composition according to [69] for use in the prevention or treatment of a disease or condition related to p53 mutant protein in a subject.

[77]. A method for preventing or treating a disease or condition related to p53 mutant protein in a subject, comprising administering to a subject a therapeutically effective amount of a compound of formula (I), or a stereoisomer, tautomer, deuterated derivative, prodrug or pharmaceutically acceptable salt thereof according to any one of [1]-[59], or a pharmaceutical composition according to [69].

[78]. The method according to [77], wherein the disease or condition is cancer.

[79]. The method according to [78], wherein the cancer cell expresses the p53 mutant. 50 [80]. The method according to [79], wherein the p53 mutant has a mutation at amino acid Val143, His168, Arg175, Tyr220, Gly245, Arg248, Arg249, Phe270, Arg273, Arg282, and/or a combination thereof.

[81]. The method according to [79], wherein the p53 mutant is V157F, R175H, Y220C, G245S, R248Q, R248W, R249S, R273H, R273C, R282W, and/or a combination thereof, preferably Y220C.

[82]. The method according to any one of [77] to [81], wherein the disease or condition is selected from the group consisting of ovarian cancer, breast cancer, lung cancer and/or a combination thereof.

### DETAILED DESCRIPTION

The present invention provides compounds, compositions and methods for restoring wild-type function of mutant p53. The compounds of the present invention can bind to mutant p53 and restore the ability of the p53 mutant to bind DNA. The restoration of activity of the p53 mutant can allow for the activation of downstream effectors of p53 leading to inhibition of cancer progression. The present invention further provides a method for treating a disease or condition related to p53 mutant protein. There is also provided a method for preparation of a compound of the present invention.

The compounds of the present invention can selectively bind to a p53 mutant and can recover wild-type activity of the p53 mutant including, for example, DNA binding function and activation of downstream targets involved in tumor suppression. In some embodiments, a compound of the invention selectively binds to the p53 Y220C mutant. The Y220C mutant is a temperature sensitive mutant, which binds to DNA at lower temperature and is denatured at body temperature. A compound of the invention can selectively bind to the p53 Y220Cs and stabilize the Y220C mutant to reduce the likelihood of denaturation of the protein at body temperature.

To determine the ability of a compound of the present invention to bind and stabilize mutant p53, assays can be utilized to detect, for example, a conformational change in the p53 mutant or activation of wild-type p53 targets. Conformational changes in p53 can be measured by, for example, differential scanning fluorimetry (DSF), isothermal titration calorimetry (ITC), nuclear magnetic resonance spectrometry (NMR), or X-ray crystallography. Additionally, antibodies specific for the wild type of mutant conformation of p53 can be used to detect a conformational change via, for example, immunoprecipitation (IP), immunofluorescence (IF), or immunoblotting. Methods used to detect the ability of the p53 mutant to bind DNA can include, for example, DNA affinity immunoblotting, modified enzyme-linked immunosorbent assay (ELISA), electrophoretic mobility shift assay (EMSA), fluorescence resonance energy transfer (FRET), homogeneous time-resolved fluorescence (HTRF), and a chromatin immunoprecipitation (ChIP) assay. To determine whether a compound described herein is able to reactivate the transcriptional activity of p53, the activation of downstream targets in the p53 signaling cascade can be measured. Activation of p53 effector proteins can be detected by, for example, immunohistochemistry (IHC—P), reverse transcription polymerase chain reaction (RT-PCR), and western blotting. The activation of p53 can also be measured by the induction of apoptosis via the caspase cascade and using methods including, for example, Annexin V staining, TUNEL assays, pro-caspase and caspase levels, and cytochrome c levels. Another consequence of p53 activation is senescence, which can be measured using methods such as β-galactosidase staining.

In an in vitro DNA binding assay, some compounds described herein can exhibit an EC₅₀ of not more than 0.010 μM, not more than 0.015 μM, not more than 0.020 μM, not more than 0.025 μM, not more than 0.030 μM, not more than 0.035 μM, not more than 0.040 μM, not more than 0.045 μM, not more than 0.050 μM, not more than 0.055 μM, not more than 0.060 μM, not more than 0.065 μM, not more than 0.070 μM, not more than 0.075 μM, not more than 0.080 μM, not more than 0.085 μM, not more than 0.090 μM, not more than 0.095 μM, not more than 0.100 μM, not more than 0.110 μM, not more than 0.120 μM, not more than 0.130 μM, not more than 0.140 μM, not more than 0.150 μM, not more than 0.160 μM, not more than 0.170 μM, not more than 0.180 μM, not more than 0.190 μM, not more than 0.200 μM, not more than 0.250 μM, not more than 0.300 μM, not more than 0.400 μM, not more than 0.500 μM, not more than 0.600 μM, not more than 0.800 μM, not more than 1.000 μM, not more than 1.500 μM, not more than 2.000 μM, not more than 6.000 μM, or not more than 10.000 μM.

In a cell viability assay for cell line harbouring p53 Y220C mutant such as NUGC-3 (p53, Y220C), some compounds described herein can exhibit an IC₅₀ of not more than 0.100 μM, not more than 0.110 μM, not more than 0.120 μM, not more than 0.130 μM, not more than 0.140 μM, not more than 0.150 μM, not more than 0.160 μM, not more than 0.170 μM, not more than 0.180 μM, not more than 0.190 μM, not more than 0.200 μM, not more than 0.250 μM, not more than 0.300 μM, not more than 0.350 μM, not more than 0.400 μM, not more than 0.450 μM, not more than 0.500 μM, not more than 0.550 μM, not more than 0.600 μM, not more than 0.650 μM, not more than 0.700 μM, not more than 0.750 μM, not more than 0.800 μM, not more than 0.850 μM, not more than 0.900 μM, not more than 0.950 μM, not more than 1.000 μM, not more than 1.500 μM, not more than 2.000 μM, not more than 2.500 μM, not more than 3.000 μM, not more than 4.000 μM, not more than 5.000 μM, not more than 6.000 μM, not more than 7.000 μM, not more than 8.000 μM, not more than 9.000 μM, not more than 15.000 μM, or not more than 25.000 μM.

In a cell viability assay for cell line with wild-type p53 such as NUGC-4 (p53, wt), some compounds described herein can exhibit an IC₅₀ of not less than 1.00 μM, not less than 1.30 μM, not less than 1.60 μM, not less than 2.00 μM, not less than 2.50 μM, not less than 3.00 μM, not less than 3.50 μM, not less than 4.00 μM, not less than 4.50 μM, not less than 5.00 μM, not less than 5.50 μM, not less than 6.00 μM, not less than 6.50 μM, not less than 7.00 μM, not less than 7.50 μM, not less than 8.00 μM, not less than 8.50 μM, not less than 9.00 μM, not less than 9.50 μM, not less than 10.00 μM, not less than 10.50 μM, not less than 11.00 μM, not less than 11.50 μM, not less than 12.00 μM, not less than 12.50 μM, not less than 13.00 μM, not less than 13.50 μM, not less than 14.00 μM, not less than 15.00 μM, not less than 16.00 μM, not less than 17.00 μM, not less than 18.00 μM, not less than 19.00 μM, not less than 20.00 μM, or not less than 21.00 μM.

In some embodiments, some compounds of the present invention can exhibit an IC₅₀ for cell line harbouring p53 Y220C mutant such as NUGC-3 (p53, Y220C) at least 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 21 times, 22 times, 23 times, 24 times, 25 times, 26 times, 27 times, 28 times, 29 times, 30 times, 31 times, 32 times, 33 times, 34 times, 35 times, 36 times, 37 times, 38 times, 39 times, 40 times, 45 times, 50 times, 55 times, 60 times, 70 times, 80 times, 90 times, 100 times, 120 times, 150 times, 200 times, or 300 times lower than that for cell line with wild-type p53 such as NUGC-4 (p53, wt).

In some embodiments, some compounds of the present invention can exhibit a selectivity or specificity for cell line harbouring p53 Y220C mutant such as NUGC-3 (p53, Y220C) of at least 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 21 times, 22 times, 23 times, 24 times, 25 times, 26 times, 27 times, 28 times, 29 times, 30 times, 31 times, 32 times, 33 times, 34 times, 35 times, 36 times, 37 times, 38 times, 39 times, 40 times, 45 times, 50 times, 55 times, 60 times, 70 times, 80 times, 90 times, 100 times, 120 times, 150 times, 200 times, or 300 times over cell line with wild-type p53 such as NUGC-4 (p53, wt).

In a reporter gene assay, some compounds of the present invention can exhibit an EC₅₀ of not more than 0.010 μM, not more than 0.015 μM, not more than 0.020 μM, not more than 0.025 μM, not more than 0.030 μM, not more than 0.035 μM, not more than 0.040 μM, not more than 0.045 μM, not more than 0.050 μM, not more than 0.055 μM, not more than 0.060 μM, not more than 0.065 μM, not more than 0.070 μM, not more than 0.075 μM, not more than 0.080 μM, not more than 0.085 μM, not more than 0.090 μM, not more than 0.095 μM, not more than 0.100 μM, not more than 0.110 μM, not more than 0.120 μM, not more than 0.130 μM, not more than 0.140 μM, not more than 0.150 μM, not more than 0.160 μM, not more than 0.170 μM, not more than 0.180 μM, not more than 0.190 μM, not more than 0.200 μM, not more than 0.250 μM, not more than 0.300 μM, not more than 0.350 μM, not more than 0.400 μM, not more than 0.450 μM, not more than 0.500 μM, not more than 0.550 μM, not more than 0.600 μM, not more than 0.650 μM, not more than 0.700 μM, not more than 0.750 μM, not more than 0.800 μM, not more than 0.900 μM, not more than 1.000 μM, not more than 5.000 μM, or not more than 10.000 μM.

The present invention is described herein using several definitions, as set forth below and throughout the application.

Unless otherwise specified or indicated by context, the terms “a”, “an”, and “the” mean “one or more.” For example, “a compound” should be interpreted to mean “one or more compounds.”

As used herein, “about,” “approximately,” “substantially,” and “significantly” will be understood by persons of ordinary skill in the art and will vary to some extent on the context in which they are used. If there are uses of these terms which are not clear to persons of ordinary skill in the art given the context in which they are used, “about” and “approximately” will mean plus or minus <10% of the particular term and “substantially” and “significantly” will mean plus or minus >10% of the particular term.

As used herein, the terms “include” and “including” have the same meaning as the terms “comprise” and “comprising” in that these latter terms are “open” transitional terms that do not limit claims only to the recited elements succeeding these transitional terms. The term “consisting of,” while encompassed by the term “comprising,” should be interpreted as a “closed” transitional term that limits claims only to the recited elements succeeding this transitional term. The term “consisting essentially of,” while encompassed by the term “comprising,” should be interpreted as a “partially closed” transitional term which permits additional elements succeeding this transitional term, but only if those additional elements do not materially affect the basic and novel characteristics of the claim.

The total number of carbon atoms present in a chemical group as defined herein is represented by a shorthand notation before the group. For example, C₁₋₆ alkyl refers to an alkyl group as defined hereinafter having 1 to 6 carbon atoms in total; C₃₋₈ cycloalkyl refers to a cycloalkyl group as defined hereinafter having 3 to 8 carbon atoms in total; C₆₋₁₀ aryl refers to an aryl group as defined hereinafter having 6 to 10 carbon atoms in total. Carbon atoms that may exist in the substituents of the chemical group are not included in the total number of carbon atoms in the shorthand notation.

Unless otherwise indicated in this specification, all combined groups according to the present invention (i.e., groups comprised of two or more groups) are attached to the rest of the molecule in such a way that the lastly described group acts as the point of attachment. By way of example, “arylalkyl” means that the aryl group is attached to the rest of the molecule via the alkyl group; “alkoxyl” means that the aliphatic group is attached to the rest of the molecule via an oxy group; etc.

In the present application, “optional” or “optionally” means that the subsequently described event or circumstance may or may not occur, and that the description includes instances where said event or circumstance occurs and instances in which it does not. Also, the term “optionally substituted” refers to any one or more hydrogen atoms on the designated atom or group may or may not be replaced by a moiety other than hydrogen. For example, “alkyl optionally substituted by one or more (such as 1, 2, 3, 4, 5 or 6) halogens” means the alkyl group is unsubstituted or substituted by one or more (such as 1, 2, 3, 4, 5 or 6) halogens, and that the description includes both substituted alkyl groups and unsubstituted alkyl groups.

The term “substituted” means that any one or more hydrogen atoms on the designated atom or group is replaced with one or more substituents other than hydrogen, provided that the designated atom's normal valence is not exceeded. The term “substituted”, unless otherwise indicated, refers to any level of substitution, e.g., mono-, di-, tri-, tetra- or penta-substitution, where such substitution is permitted. The substituents are independently selected, and substitution may be at any chemically accessible position. It is to be understood that substitution at a given atom is limited by valency. It is to be understood that substitution at a given atom results in a chemically stable molecule. As used herein, the term “substituted” is contemplated to include all permissible substituents of organic compounds. In abroad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. The phrase “optionally substituted” means unsubstituted or substituted. The term “substituted” means that a hydrogen atom is removed and replaced by a substituent. A single divalent substituent, e.g., oxo, can replace two hydrogen atoms.

The term “stereoisomer” refers to a compound made up of the same atoms bonded by the same bonds but having different three-dimensional structures. All the stereoisomers of the invention can be identified and determined by conventional X-ray single crystal diffraction analysis. The present invention contemplates various stereoisomers and mixtures thereof.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds described herein may contain asymmetric or chiral centers, and therefore exist in different stereoisomeric forms. Many organic compounds exist in optically active forms, i.e., they have the ability to rotate the plane of plane-polarized light. In describing an optically active compound, the prefixes D and L, or R and S, are used to denote the absolute configuration of the molecule about its chiral center (s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

The term “tautomer” refers to an isomer resulted from a proton shift from one atom of a molecule to another atom of the same molecule. All tautomeric forms of the compound of formula (I) of the present invention are included within the scope of the present invention.

Unless otherwise indicated, structures depicted herein are also meant to include all isomeric forms of the structure, e.g., racemic mixtures, cis/trans isomers, geometric (or conformational) isomers, such as (Z) and (E) isomers. Unless specified otherwise, the compound having a double bond or cycle in the present application includes both E- and Z-geometric isomers.

Unless specified otherwise, the bond “ [symbol] ” or “ [symbol] ” in this application includes “ [symbol] ” and “ [symbol] ”.

All isotopes of any particular atom or element as specified are contemplated within the scope of the compounds of the invention and their uses. Isotopes include those atoms having the same atomic number but different mass numbers. Exemplary isotopes that can be incorporated into compounds of the invention include isotopes of hydrogen, carbon, nitrogen, oxygen, phosphorus, sulfur, fluorine, chlorine and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I or ¹²⁵I. By way of general example and without limitation, isotopes of hydrogen include deuterium and tritium. The isotopes of hydrogen can be denoted as ¹H(hydrogen), ²H(deuterium) and ³H(tritium). They are also commonly denoted as D for deuterium and T for tritium. In the application, CD₃ denotes a methyl group wherein all of the hydro gen atoms are deuterium. Isotopes of carbon include ¹³C and ¹⁴C. Isotopically labeled compounds of the present disclosure are equivalent to those unlabeled, for example, deuterated compounds of the present disclosure are equivalent to those non-deuterated. Isotopically-labeled compounds of the invention can generally be prepared by conventional techniques known to those skilled in the art or by processes analogous to those described herein, using an appropriate isotopically-labeled reagent in place of the non-labeled reagent.

As used herein, “deuterated derivative” refers to a compound having the same chemical structure as a reference compound, but with one or more hydrogen atoms replaced by a deuterium atom (“D” or “2H”). It will be recognized that some variation of natural isotopic abundance occurs in a synthesized compound depending on the origin of chemical materials used in the synthesis. Notwithstanding this variation, the concentration of naturally abundant stable hydrogen isotopes is small and immaterial as compared to the degree of stable isotopic substitution of deuterated derivatives described herein. Thus, unless otherwise stated, when a reference is made to a “deuterated derivative” of a compound of the disclosure, at least one hydrogen is replaced with deuterium at a level that is well above its natural isotopic abundance, which is typically about 0.015%. In some embodiments, the deuterated derivatives disclosed herein have an isotopic enrichment factor for each deuterium atom, of at least 3500 (52.5% deuterium incorporation at each designated deuterium), at least 4500 (67.5% deuterium incorporation at each designated deuterium), at least 5000 (75% deuterium incorporation at each designated deuterium), at least 5500 (82.5% deuterium incorporation at each designated deuterium), at least 6000 (90% deuterium incorporation at each designated deuterium), at least 6333.3 (95% deuterium incorporation at each designated deuterium), at least 6466.7 (97% deuterium incorporation at each designated deuterium), or at least 6600 (99% deuterium incorporation at each designated deuterium). The term “isotopic enrichment factor” as used herein means the ratio between the isotopic abundance and the natural abundance of a specified isotope.

In addition to the above-mentioned, as used in the specification and claims, unless otherwise indicated, the following terms have the meanings as set forth below:
“amino” refers to the —NH₂ group.
“cyano” refers to the —CN group.
“hydroxy” refers to the —OH group
“nitro” refers to the —NO₂ group.
“carboxyl” refers to the —COOH group.
“nitroso” refers to the —N═O group.

The term “halogen”, as used herein, unless otherwise indicated, means fluoro, chloro, bromo or iodo. The preferred halogen groups include —F, —Cl and —Br.

The term “alkyl”, as used herein, unless otherwise indicated, includes saturated monovalent hydrocarbon radicals having straight or branched. For example, alkyl radicals include methyl, ethyl, propyl, isopropyl, n-butyl, isobutyl, sec-butyl, t-butyl, n-pentyl, 3-(2-methyl)butyl, 2-pentyl, 2-methylbutyl, neopentyl, n-hexyl, 2-hexyl and 2-methylpentyl. Similarly, C₁₋₆, as in C₁₋₆alkyl is defined to identify the group as having 1, 2, 3, 4, 5 or 6 carbon atoms in a linear or branched arrangement.

The term “alkenyl” means a straight or branch-chained hydrocarbon radical containing one or more (such as 1, 2, 3, 4, 5 or 6) double bonds and typically from 2 to 20 carbon atoms in length. For example, “C₂₋₆alkenyl” contains from 2 to 6 carbon atoms. Alkenyl group include, but are not limited to, for example, ethenyl, propenyl, butenyl, 2-methyl-2-buten-1-yl, hepetenyl, octenyl and the like.

The term “alkynyl” contains a straight or branch-chained hydrocarbon radical containing one or more (such as 1, 2, 3, 4, 5 or 6) triple bonds and typically from 2 to 20 carbon atoms in length. For example, “C₂₋₆alkynyl” contains from 2 to 6 carbon atoms. Representative alkynyl groups include, but are not limited to, for example, ethynyl, 1-propynyl, 1-butynyl, heptynyl, octynyl and the like.

The term “alkoxyl” radicals are oxygen ethers formed from the previously described alkyl groups.

The term “oxo” refers to the group ═O or (O), or an oxygen atom attached to other atom (e.g. C, N, S, or P) via a double bond.

A “cycloalkyl” is a cyclic hydrocarbon which is completely saturated. “cycloalkyl” includes monocyclic and bicyclic rings. Typically, a monocyclic cycloalkyl has from 3 to about 12 carbon atoms, more typically 3 to 8 carbon atoms unless otherwise defined, exemplary “cycloalkyl” groups includes but not limited to, cyclopropyl, cyclobutyl, cyclopentyl, cyclohexyl and so on. Cycloalkyl includes bicyclic molecules in which one, two or three or more atoms are shared between the two rings. The term “spirocyclic cycloalkyl” refers to a bicyclic cycloalkyl in which each of the rings shares one adjacent atom with the other ring. The term “fused cycloalkyl” refers to a bicyclic cycloalkyl in which each of the rings shares two adjacent atoms with the other ring. The term “bridged cycloalkyl” refers to a cycloalkyl that includes at least two bridgehead carbon atoms and at least one bridging carbon atom. “bridged cycloalkyl” includes “bicyclic bridged cycloalkyls” which includes two bridgehead carbon atoms and “polycyclic bridged cycloalkyls” which includes more than two bridgehead carbon atoms. Typical bridged cycloalkyls include, but are not limited to adamantyl, noradamantyl, bicyclo[1.1.0]butanyl, norboranyl (bicyclo[2.2.1]heptanyl), norbornenyl(bicyclo[2.2.1]heptanyl), norbornadienyl (bicyclo[2.2.1]heptadienyl), tricyclo[2.2.1.0]heptanyl, bicyclo[3.2.1]octanyl, bicyclo[3.2.1]octanyl, bicyclo[3.2.1]octadienyl, bicyclo[2.2.2]octanyl, bicyclo[2.2.2]octenyl, bicyclo[2.2.2]octadienyl, bicyclo[5,2,0]nonanyl, bicyclo[4.3.2]undecanyl, tricyclo[5.3.1.1]dodecanyl, and the like.

The term “cycloalkenyl” refers to a partially unsaturated cyclic hydrocarbon group containing 1 to 4 rings and 3 to 8 carbons per ring system. Exemplary such groups include cyclobutenyl, cyclopentenyl, cyclohexenyl, etc. “cycloalkenyl” includes monocyclic, bicyclic, tricyclic, or tetracyclic ring system in which one, two, three or more atoms are shared between the two rings. The term “spirocyclic cycloalkenyl” refers to a bicyclic cycloalkenyl in which each of the rings shares one adjacent atom with the other ring. The term “fused cycloalkenyl” refers to a polycyclic cycloalkenyl in which two rings shares two adjacent atoms. The term “bridged cycloalkenyl” refers to a cycloalkenyl that includes at least two bridgehead atoms and at least one bridging atom. “bridged cycloalkenyl” includes “bicyclic bridged cycloalkenyl” which includes two bridgehead atoms and “polycyclic bridged cycloalkenyl” which includes more than two bridgehead atoms.

The term “heterocycloalkyl” refers to a stable 3-18 membered non-aromatic ring radical completely saturated that comprises two to twelve carbon atoms and from one to six heteroatoms selected from such as nitrogen, oxygen, phosphorus and sulfur. Unless stated otherwise specifically in the specification, the hetero cycloalkyl radical is a monocyclic, bicyclic, tricyclic, or tetracyclic ring system. The term “spirocyclic heterocycloalkyl” or “spiro-heterocyclyl” refers to a polycyclic heterocycloalkyl in which two rings shares one atom. The term “fused heterocycloalkyl” refers to a polycyclic heterocycloalkyl in which two rings shares two adjacent atoms. The term “bridged heterocycloalkyl” or “bridged-heterocyclyl” refers to a heterocycloalkyl that includes at least two bridgehead atoms and at least one bridging atom. “Bridged heterocycloalkyl” or “bridged-heterocyclyl” includes “bicyclic bridged heterocycloalkyl” which includes two bridgehead atoms and “polycyclic bridged heterocycloalkyl” which includes at least two bridgehead atoms. The heteroatoms in the heterocycloalkyl radical are optionally oxidized. In some embodiments, the heterocycloalkyl is attached to the rest of the molecule through any atom of the ring(s). Examples of heterocycloalkyl radicals include, but are not limited to, dioxolanyl, thienyl[1,3] dithianyl, decahydroisoquinolyl, imidazolinyl, imidazolidin yl, isothiazolidinyl, isoxazolidinyl, morpholinyl, octahydroindolyl octahydroisoindoly, 2-oxopiperazinyl, 2-oxo piperidinyl, 2-oxopyrrolidinyl, oxazolidinyl, piperidnyl, piperazinyl, 4-piperidonyl, pyrrolidinyl, pyrazolidinyl quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl.

The term “heterocycloalkenyl” refers to the above-mentioned hererocycloalkyl having at least one double bond. A heterocycloalkenyl may be a single ring or multiple rings wherein the multiple rings include “spirocyclic heterocycloalkenyl”, “fused heterocycloalkenyl”, and “bridged heterocycloalkenyl”. “Spirocyclic heterocycloalkenyl” refer to a polycyclic heterocycloalkenyl in which two rings shares one atom, “fused hetero cycloalkenyl” refers to a polycyclic heterocycloalkenyl in which two rings shares two adjacent atoms, and “bridged heterocycloalkyl” refers to a heterocycloalkenyl that includes at least two bridgehead atoms and at least one bridging atom. “Bridged heterocycloalkenyl” includes “bicyclic bridged heterocycloalkenyl” which includes two bridgehead atoms and “polycyclic bridged heterocycloalkenyl” which includes more than two bridgehead atoms.

The term “aryl”, as used herein, unless otherwise indicated, refers to an unsubstituted or substituted mono or polycyclic aromatic ring system containing carbon ring atoms. The preferred aryls are mono cyclic or bicyclic aromatic ring systems. Phenyl and naphthyl are preferred aryls.

The term “heteroaryl”, as used herein, unless otherwise indicated, represents an aromatic ring system containing carbon(s) and at least one heteroatom. Heteroaryl may be monocyclic or polycyclic, substituted or unsubstituted. A monocyclic heteroaryl group may have 1 to 4 heteroatoms in the ring, while a polycyclic heteroaryl may contain 1 to 10 hetero atoms. A polycyclic heteroaryl ring may contain fused, spiro or bridged ring junction, for example, bicyclic heteroaryl is a polycyclic heteroaryl. Bicyclic heteroaryl rings may contain from 8 to 12 ring atoms. Monocyclic heteroaryl rings may contain from 5 to 8 ring atoms (carbons and heteroatoms). Examples of heteroaryl groups include, but are not limited to thienyl, furanyl, imidazolyl, isoxazolyl, oxazolyl, pyrazolyl, pyrrolyl, thiazolyl, thiadiazolyl, triazolyl, pyridyl, pyridazinyl, indolyl, azaindolyl, indazolyl, benzimidazolyl, benzofuranyl, benzothienyl, benzisoxazolyl, benzoxazolyl, benzopyrazolyl, benzothiazolyl, benzothiadiazolyl, benzotriazolyl, adeninyl, quinolinyl or isoquinolinyl.

The term “heterocyclyl” or “heterocycle” as used herein refers to a single saturated or partially unsaturated non-aromatic ring or a non-aromatic multiple ring system that has at least one heteroatom in the ring (e.g., at least one annular heteroatom selected from oxygen, nitrogen, phosphorus and sulfur). Unless other wise specified, a heterocyclyl group has from 3 to about 20 annular atoms, for example from 3 to 12 annular atoms, for example from 3 to 10 annular atoms, for example from 5 to 10 annular atoms or for example from 5 to 6 annular atoms. Thus, the term includes single saturated or partially unsaturated rings (e.g., 3, 4, 5, 6 or 7-membered rings) having from about 1 to 6 annular carbon atoms and from about 1 to 3 annular heteroatoms selected from the group consisting of oxygen, nitrogen and sulfur in the ring. The rings of the multiple condensed ring (e.g. bicyclic heterocyclyl) system can be connected to each other via f used, spiro and bridged bonds when allowed by valency requirements. The term “heterocyclyl” or “heterocyclic ring” or “heterocycle” includes heterocycloalkenyl groups (i.e., the heterocyclyl group having at least one double bond). A heterocyclyl may be a single ring or multiple rings wherein the multiple rings may be fused, bridged, or spiro. As used herein, heterocyclyl has 2 to 20 ring carbon atoms (i.e., C₂₋₂₀heterocycle), 2 to 12 ring carbon atoms (i.e., C₂₋₁₂ heterocyclyl), 2 to 10 ring carbon atoms (i.e., C₂₋₁₀ heterocyclyl), 2 to 8 ring carbon atoms (i.e., C₂₋₈heterocyclyl), 3 to 12 ring carbon atoms (i.e., C₃₋₁₂ heterocyclyl), 3 to 8 ring g carbon atoms (i.e., C₃₋₈ heterocyclyl), or 3 to 6 ring carbon atoms (i.e., C₃₋₆ heterocyclyl); having 1 to 5 ring heteroatoms, 1 to 4 ring heteroatoms, 1 to 3 ring heteroatoms, 1 to 2 ring heteroatoms, or 1 ring hetero atom independently selected from nitrogen, sulfur or oxygen. Examples of heterocyclyl groups include pyrrolidinyl, piperidinyl, piperazinyl, oxetanyl, dioxolanyl, azetidinyl, and morpholinyl. As used herein, the term “bridged-heterocyclyl” refers to a four- to ten-membered cyclic moiety connected at two non-adjacent atoms of the heterocyclyl with one or more (e.g., 1 or 2) four- to ten-membered cyclic moiety having at least one heteroatom where each heteroatom is independently selected from nitrogen, oxygen, and sulfur. As used herein, “bridged-heterocyclyl” includes bicyclic and tricyclic ring systems. Also as used herein, the term “spiro-heterocyclyl” refers to a ring system in which a three- to ten-membered heterocyclyl has one or more additional ring, wherein the one or more additional ring is three- to ten-membered cycloalkyl or three- to ten-membered heterocyclyl, where a single atom of the one or more additional ring is also an atom of the three- to ten-membered heterocyclyl. Examples of the spiro-heterocyclyl include bicyclic and tricyclic ring systems, such as 2-oxa-7-azaspiro[3.5]nonanyl, 2-oxa-6-azaspiro[3.4]octanyl, and 6-oxa-1-azaspiro[3.3]heptanyl. Heterocyclyl groups also include partially unsaturated ring systems containing one or more double bonds, including fused ring systems with one aromatic ring and one non-aromatic ring, but not fully aromatic ring systems. Examples include dihydroquinolines (e.g. 3,4-dihydroquinoline), dihydroisoquinolines (e.g. 1,2-dihydroisoquinoline), dihydroimidazole, tetrahydroimidazole, indoline, isoindoline, isoindolones (e.g. isoindolin-1-one), isatin, dihydrophthalazine, quinolinone, spiro[cyclopropane-1,1′-isoindolin]-3′-one, tetrahydroisoquinoline, tetraline, and the like. Additional examples of heterocycles include 3,8-diazabicyclo[3.2.1]octanyl, 2,5-diazabicyclo[2.2.1]heptanyl, 3,6-diazabicyclo[3.1.1]heptanyl, 3-oxa-7,9-diazabicyclo[3.3.1]nonanyl, and hexahydropyrazino[2,1-c][1,4]oxazinyl, for example. As used herein, the terms “heterocycle”, “heterocyclyl”, and “heterocyclic ring” are used interchangeably.

Any hydrogen atom bonded with C, N, O, or S in the 3-12 membered cycloalkyl, 3-12 membered cycloalkenyl, 3-12 membered heterocycloalkyl, 3-12 membered heterocycloalkenyl, 3-12 membered heterocyclyl, 6-10 membered aryl or 5-12 membered heteroaryl can be replaced with the substituent.

The further optional reduction, oxidation or other functionalization of compounds of formula (I) of the invention may be carried out according to methods well known to those skilled in the art. Within the scope of this text, only a readily removable group that is not a constituent of the particular desired end product of the compounds of the present invention is designated a “protecting group”, unless the context indicates otherwise. The protection of functional groups by such protecting groups, the protecting groups themselves, and their cleavage reactions are described for example in standard reference works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of Organic Chemistry), Houben Weyl, 4th edition, Volume 15/I, Georg Thieme Verlag, Stuttgart 1974, and in H.-D. Jakubke and H. Jeschkeit, “Aminosauren, Peptide, Proteine” (Amino acids, Peptides, Proteins), Verlag Chemie, Weinheim, Deerfield Beach, and Basel 1982. A characteristic of protecting groups is that they can be removed readily (i.e. without the occurrence of undesired secondary reactions) for example by solvolysis, reduction, photolysis or alternatively under physiological conditions (e.g. by enzymatic cleavage).

The term “leaving group” as used herein has the meaning conventionally defined in synthetic organic chemistry, i.e., an atom or a group capable of being displaced by a nucleophile and includes halo (such as chloro, bromo, and iodo), alkanesulfonyloxy, arenesulfonyloxy, alkylcarbonyloxy (e.g., acetoxy), arylcarbonyloxy, mesyloxy, tosyloxy, trifluoromethanesulfonyloxy, aryloxy (e.g., 2,4-dinitrophenoxy), methoxy, N, O-dimethylhydroxylamino, and the like.

The term “reductive amination reaction” as used herein has the meaning generally understood in the art, and it can be carried out in the presence of a reductive amination agent by a person skilled in the art. In some embodiments, the reductive amination agent used in a reductive amination reaction is selected from the group consisting of sodium cyanoborohydride, sodium triacetoxy borohydride, sodium borohydride, organic borane complex compounds such as a 4-(dimethylamino)pyridine borane complex, N-ethyldiisopropylamine borane complex, N-ethylmorpholine borane complex, N-methylmorpholine borane complex, N-phenylmorpholine borane complex, lutidine borane complex, triethylamine borane complex, trimethylamine borane complex, and a combination of two or more thereof. Preferably, the reductive amination agent is sodium cyanoborohydride (NaBH₃CN).

The term “composition”, as used herein, is intended to encompass a product comprising the specified ingredients in the specified amounts, as well as any product which results, directly or indirectly, from combinations of the specified ingredients in the specified amounts. Accordingly, pharmaceutical compositions containing the compounds of the present invention as the active ingredient as well as methods of preparing the instant compounds are also part of the present invention. Furthermore, some of the crystalline forms for the compounds may exist as polymorphs and as such are intended to be included in the present invention. In addition, some of the compounds may form solvates with water (i.e., hydrates) or common organic solvents and such solvates are also intended to be encompassed within the scope of this invention.

When the compound and pharmaceutically acceptable salts thereof exist in the form of solvates or polymorphic forms, the present invention includes any possible solvates and polymorphic forms. A type of a solvent that forms the solvate is not particularly limited so long as the solvent is pharmacologically acceptable. For example, water, ethanol, propanol, acetone or the like can be used.

In many cases, the compounds of this disclosure are capable of forming acid addition salt and/or base addition salt by virtue of the presence of amino and/or carboxyl groups or groups similar thereto.

The term “pharmaceutically acceptable salt” of a given compound refers to salts that retain the biological effectiveness and properties of the given compound, and which are not biologically or otherwise undesirable. Pharmaceutically acceptable base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, mono, di or tri cycloalkyl amines, mono, di or tri arylamines or mixed amines, etc. Specific examples of suitable amines include, by way of example only, isopropylamine, trimethylamine, diethylamine, tri(iso-propyl)amine, tri(n-propyl)amine, ethanolamine, 2-dimethylaminoethanol, piperazine, piperidine, morpholine, N-ethylpiperidine, and the like. Pharmaceutically acceptable acid addition salts may be prepared from inorganic and organic acids. Salts derived from inorganic acids include salts of hydrochloric acid, hydrobromic acid, sulfuric acid, nitric acid, phosphoric acid, and the like. Salts derived from organic acids include salts of acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Since the compounds are intended for pharmaceutical use, they are preferably provided in substantially pure form, for example at least 60% pure, more suitably at least 75% pure, especially at least 98% pure (% are on a weight for weight basis).

The term “prodrug” refers to a biologically inactive derivative of a drug that upon administration to the human body is converted to the biologically active parent drug according to some chemical or enzymatic pathway.

As used herein, the term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

The pharmaceutical compositions of the present invention comprise a compound (or a pharmaceutically acceptable salt thereof) as an active ingredient, a pharmaceutically acceptable carrier and optionally other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds or a prodrug or a metabolite or pharmaceutically acceptable salts thereof, of this invention can be combined as the active ingredient in intimate admixture with a pharmaceutical carrier according to conventional pharmaceutical compounding techniques. The carrier may take a wide variety of forms depending on the form of preparation desired for administration, e.g. oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compound or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more (such as 1, 2, 3, 4, 5 or 6) necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and the above-mentioned compound or a pharmaceutically acceptable salt thereof. The compounds or pharmaceutically acceptable salts thereof, can also be included in pharmaceutical compositions in combination with one or more (such as 1, 2, 3, 4, 5 or 6) other therapeutically active compounds.

A tablet containing the composition of this invention may be prepared by compression or molding, optionally with one or more (such as 1, 2, 3, 4, 5 or 6) accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient. For example, a formulation intended for the oral administration to humans may contain from about 0.5 mg to about 5 g of active agent, compounded with an appropriate and convenient amount of carrier material which may vary from about 0.05 to about 95 percent of the total composition. Unit dosage forms will generally contain between from about 0.01 mg to about 2 g of the active ingredient, typically 0.01 mg, 0.02 mg, 1 mg, 2 mg, 3 mg, 4 mg, 5 mg, 6 mg, 7 mg, 8 mg, 9 mg, 10 mg, 25 mg, 50 mg, 100 mg, 200 mg, 300 mg, 400 mg, 500 mg, 600 mg, 800 mg or 1000 mg.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder or the like. Further, the compositions can be in a form suitable for use in transdermal devices. These formulations may be prepared, utilizing a compound of this invention or a pharmaceutically acceptable salt thereof, via conventional processing methods. As an example, a cream or ointment is prepared by admixing hydrophilic material and water, together with about 0.05 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first mixing the composition with the softened or melted carrier(s) followed by chilling and shaping in molds.

In addition to the aforementioned carrier ingredients, the pharmaceutical formulations described above may include, as appropriate, one or more (such as 1, 2, 3, 4, 5 or 6) additional carrier ingredients such as diluents, buffers, flavoring agents, binders, surface-active agents, thickeners, lubricants, preservatives (including antioxidants) and the like. Furthermore, other adjuvants can be included to render the formulation isotonic with the blood of the intended recipient. Compositions containing a compound or pharmaceutically acceptable salts thereof, may also be prepared in powder or liquid concentrate form.

Generally, dosage levels on the order of from about 0.001 mg/kg to about 150 mg/kg of body weight per day are useful in the treatment of the above-indicated conditions or alternatively about 0.05 mg to about 7 g per patient per day. For example, inflammation, cancer, psoriasis, allergy/asthma, disease and conditions of the immune system, disease and conditions of the central nervous system (CNS), may be effectively treated by the administration of from about 0.001 to 50 mg of the compound per kilogram of body weight per day or alternatively about 0.05 mg to about 3.5 g per patient per day.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

In some embodiments, disclosed herein is a method of treating a cancer, the method comprising administering to a subject in need thereof a therapeutically-effective amount of a compound of the invention. A compound of the invention can, for example, slow the proliferation of cancer cell lines, or kill cancer cells. Non-limiting examples of cancer that can be treated by a compound of the invention include: acute lymphoblastic leukemia, acute myeloid leukemia, adrenocortical carcinoma, AIDS-related cancers, AIDS-related lymphoma, anal cancer, bladder cancer, bone cancers, brain tumors, such as cerebellar astrocytoma, cerebral astrocytoma/malignant glioma, ependymoma, medulloblastoma, supratentorial primitive neuroectodermal tumors, visual pathway and hypothalamic glioma, breast cancer, central nervous system lymphoma, cerebellar astrocytoma, cervical cancer, colon cancer, gallbladder cancer, gastric cancer, head and neck cancer, heart cancer, hepatocellular (liver) cancer, kidney cancer, liver cancer, lung cancers, such as non-small cell and small cell lung cancer, ovarian cancer, ovarian epithelial cancer, ovarian germ cell tumor, pancreatic cancer, pancreatic cancer islet cell, prostate cancer, rectal cancer, renal cell carcinoma, skin cancers, skin carcinoma merkel cell, small intestine cancer, and throat cancer.

### EXAMPLES

### Methods of Preparation

The following examples have been included to provide guidance to one of ordinary skill in the art for practicing representative embodiments of the presently disclosed subject matter. In light of the present disclosure and the general level of skill in the art, those of skill can appreciate that the following examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner the present invention.

All parts and percentages are by weight and all temperatures are degrees Celsius, unless explicitly stated otherwise.

The following abbreviations have been used in the examples:

| Abbreviation | Meaning |
|---|---|
| EA | Ethyl acetate |
| DIEA | N,N-Diisopropylethylamine |
| EtOH | Ethanol |
| DMSO | Dimethyl sulfoxide |
| ACN | Acetonitrile |
| PPA | Polyphosphoric acid |
| NIS | N-Iodosuccinimide |

### General Synthetic Schemes 1:

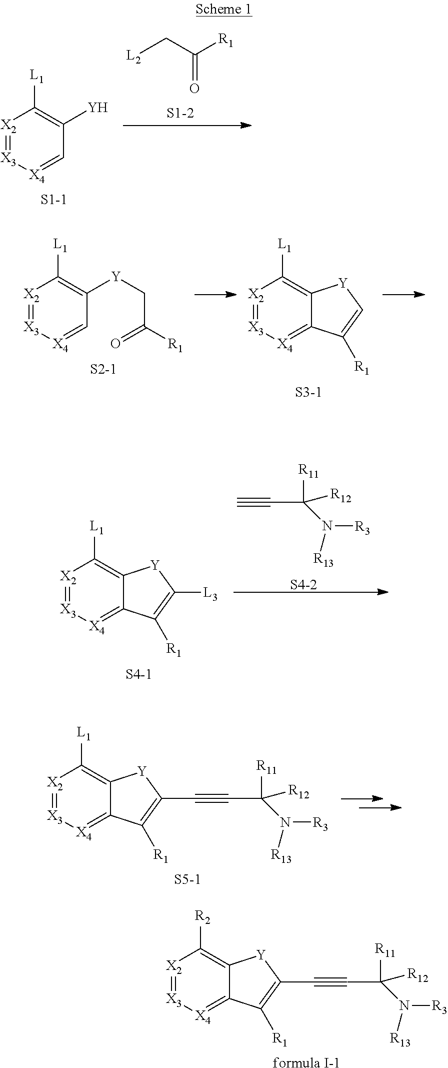

As exemplified in general synthetic scheme 1, the compound of formula (I-1) can be synthesized by the following steps:
(1) the compound with —YH group and L₁ group such as S1-1 as starting material can be reacted with the compound such as S1-2 in the presence of an alkaline agent such as K₂CO₃ to form the compound of S2-1; (2) A cyclization reaction of the compound of S2-1 and subsequent aromatization reaction can be occurred to form compound S3-1 under acid condition such as polyphosphoric acid;
(3) The adjacent position of Y atom of compound S3-1 can be halogenated in the presence of halogenating reagents to afford compound S4-1. For example, the adjacent position of Y atom of compound S3-1 can be iodated in the presence of NIS.
(4) The compound of S5-1 can be provided by reacting compound S4-1 with reagent S4-2 through a coupling reaction in the presence of a coupling catalyst, which preferably comprises Pd-containing catalyst such as Pd(PPh₃)₂Cl₂ or Pd(dppf)Cl₂;
(5) The L₁ group of the compound S5-1 can be converted to the target compound of formula (I-1) through one or more steps of reaction, for example, the compound S5-1 can be substituted with —NH₂ to generate a compound which can be converted to the target compound through a reductive amination reaction when R₂ is —NR₅₁R₅₂.
L₁, L₂ and L₃ in the general synthetic schemes 1 independently represent a leaving group such as halogen (such as —Cl, —Br or —I). Preferably, L₁ is selected from —Br; L₂ is selected from —Br; and L₃ is selected from —I.
Y in the general synthetic scheme 1 is selected from O or S;

The other variables such as X₂, X₃, X₄, R₁, R₂, R₁₁, R₁₂, R₁₃, R₃, R₅₁, R₅₂ and m are each defined as above in the present invention.

### X-Ray Single Crystal Diffraction Analysis

All the stereoisomers of the invention can be identified and determined by X-ray single crystal diffraction analysis.

Generally, the structure of a single crystal of a compound of the invention is analyzed by Olex2 (version: 1.5) software, and the diffraction data is preliminarily analyzed by ShelXT (version: 2018/2) preliminary solution program (using the intrinsic phasing method), and the space group of the single crystal can be determined. Then, the full matrix least squares structure refinement based on F2 is carried out by using the refinement program of ShelXL (version: 2018/3). All non-hydrogen atoms are anisotropically refined. Hydrogen atoms are refined by theoretical hydrogenation (riding model).

### Example 1

### 4-((3-(3-ethyl-7-((1-methylpiperidin-4-yl)amino)benzo[b]thiophen-2-yl)prop-2-yn-1-yl)amino)benzenesulfonamide (1)

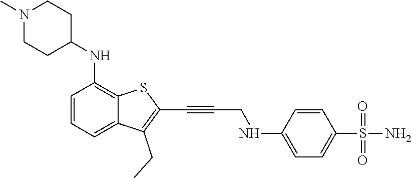 [SMILES: CCc1c(C#CCNc2ccc(S(N)(=O)=O)cc2)sc2c(NC3CCN(C)CC3)cccc12]

### Step 1. Synthesis of 1-((2-bromophenyl)thio)butan-2-one

To a solution of 2-bromobenzenethiol (16.58 g, 0.09 mol) in 160 ml ACN, K₂CO₃ (24.42 g, 0.18 mol) and 1-bromobutan-2-one (14.60 g, 0.10 mol) were added. The mixture was stirred at room temperature for 2 hours and the solid was removed by filtration. The filtrate was concentrated under vacuum. The residue was applied onto a silica gel column eluted with EA/hexane (v/v=0%-12%). This resulted in 19.89 g of 1-((2-bromophenyl)thio)butan-2-one as yellow oil. LCMS: m/z=259 [M+1]⁺.

### Step 2. Synthesis of 7-bromo-3-ethylbenzo[b]thiophene

A mixture of polyphosphoric acid (10 mL) and 1-((2-bromophenyl)thio)butan-2-one (1.99 g, 7.68 mmol) was slowly heated to 160° C. with continuous stirring. The reaction mixture was stirred for 2 hours at 160° C. Then the mixture was cooled and water (30 mL) was added. The mixture was extracted with EA (90 mL×3), dried over (Na₂SO₄), filtered, and concentrated under vacuum. The residue was applied onto a silica gel column eluted with EA/hexane (v/v=0%-5%). This resulted in 1.334 g of 7-bromo-3-ethylbenzo[b]thiophene as yellow oil.

### Step 3. Synthesis of 7-bromo-3-ethyl-2-iodobenzo[b]thiophene

To a solution of 7-bromo-3-ethylbenzo[b]thiophene (1.093 g, 4.53 mmol) in 10 mL AcOH, NIS (1.307 g, 5.81 mmol) was added in portion wise. The mixture was stirred at room temperature for 2 hours. The reaction was quenched by the addition of water (20 mL). The resulting solution was extracted with EA (2×30 mL). Then the organic layers were combined, washed with brine (15 mL), dried over anhydrous Na₂SO₄ and concentrated under vacuum. The residue was applied onto a silica gel column eluted with EA/hexane (v/v=0%-5%). This resulted in 1.186 g of 7-bromo-3-ethyl-2-iodobenzo[b]thiophene as yellow oil.

### Step 4. Synthesis of 4-((3-(7-bromo-3-ethylbenzo[b]thiophen-2-yl)prop-2-yn-1-yl)amino) benzenesulfonamide

Into a 20-mL sealed tube purged and maintained with an inert atmosphere of nitrogen, was placed 7-bromo-3-ethyl-2-iodobenzo[b]thiophene (0.504 g, 1.37 mmol), 4-(prop-2-yn-1-ylamino)benzenesulfonamide (0.335 g, 1.59 mmol), Pd(PPh₃)₂Cl₂ (0.219 g, 0.31 mmol), CuI (0.093 g, 0.49 mmol), DIEA (0.529 g, 4.09 m mol), DMSO (5 mL). The reaction mixture was stirred at room temperature for 1 h. The residue was applied onto a silica gel column eluted with EA/hexane (v/v=0%-35%). This resulted in 0.424 g of 4-((3-(7-bromo-3-ethylbenzo[b]thiophen-2-yl) prop-2-yn-1-yl)amino)benzenesulfonamide as yellow oil. LCMS: m/z=449 [M+1]⁺.

### Step 5. Synthesis of 4-((3-(7-amino-3-ethylbenzo[b]thiophen-2-yl)prop-2-yn-1-yl)amino) benzenesulfonamide

Into a 20-mL sealed tube purged and maintained with an inert atmosphere of nitrogen, was placed 4-((3-(7-bromo-3-ethylbenzo[b]thiophen-2-yl)prop-2-yn-1-yl)amino)benzenesulfonamide (0.402 g, 0.89 mmol), Na N₃ (0.135 g, 2.08 mmol), L-Proline (0.038 g, 0.33 mmol), CuI (0.042 g, 0.22 mmol), NaI (0.145 g, 0.97 mmol), Cs₂CO₃ (0.586 g, 1.80 mmol), DMSO (5 mL). The reaction mixture was stirred at 100° C. for 5 h. The residue was purified by Prep-HPLC CH₃CN/H₂O (0.2% HCOOH)(v/v=0%-50%). This resulted in 0.048 g of 4-((3-(7-amino-3-ethylbenzo[b]thiophen-2-yl)prop-2-yn-1-yl)amino) benzenesulfonamide as yellow solid. LCMS: m/z=389 [M+1]⁺.

### Step 6. Synthesis of 4-((3-(3-ethyl-7-((1-methylpiperidin-4-yl)amino)benzo[b]thiophen-2-yl) prop-2-yn-1-yl)amino)benzenesulfonamide (1)

To a solution of 4-((3-(7-amino-3-ethylbenzo[b]thiophen-2-yl)prop-2-yn-1-yl)amino) benzenesulfonamide (0.040 g, 4.53 mmol) in 5 ml MeOH, 1-methylpiperidin-4-one (0.087 g, 768.8419 μmol) was added. The mixture was stirred at room temperature for 0.5 hours. Then NaBH₃CN (0.087 g 2.03 mmol) and AcOH (0.002 mL) were added to the system. The reaction mixture was stirred at room temperature for 3 days. The residue was purified by Prep-HPLC CH₃CN/H₂O (0.05% NH₃H₂O)(v/v=0%-50%). This resulted in 0.010 g (17%) of 4-((3-(3-ethyl-7-((1-methylpiperidin-4-yl)amino)benzo[b]thiophen-2-yl)prop-2-yn-1-yl)amino)benzenesulfonamide (1) as white solid. LCMS: m/z=483 [M+1]⁺.

¹H NMR (400 MHz, CD₃OD) δ 7.71-7.65 (m, 2H), 7.22-7.15 (m, 1H), 7.11-7.03 (m, 1H), 6.84-6.73 (m, 2H), 6.68-6.59 (m, 1H), 4.29-4.20 (m, 2H), 3.49-3.40 (m, 1H), 2.84-2.73 (m, 4H), 2.30 (s, 3H), 2.21-2.13 (m, 2H), 2.03-1.95 (m, 2H), 1.61-1.51 (m, 2H), 1.14 (s, 3H).

### Example 2

### (Z)-3-fluoro-N-(2-(3-((2-methoxy-4-(methylsulfonyl)phenyl)amino)prop-1-yn-1-yl)-3-(2,2,2-trifluoroethyl)benzo[b]thiophen-7-yl)-1-methylpiperidin-4-amine (racemic) (2)

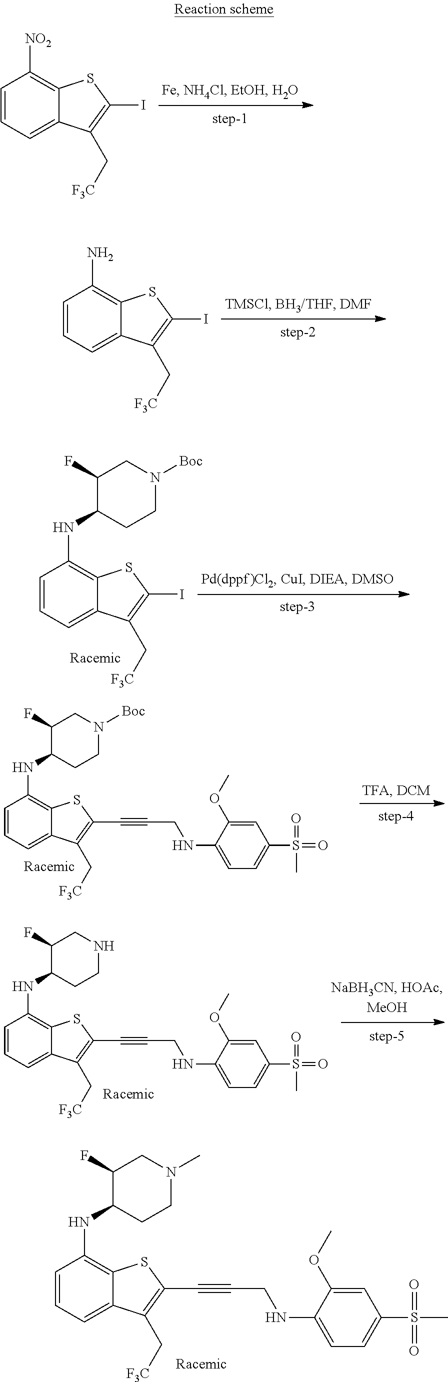

### Experimental Details

### Step 1. 2-iodo-3-(2,2,2-trifluoroethyl)benzo[b]thiophen-7-amine

Into a 20-mL sealed tube was placed 2-iodo-7-nitro-3-(2,2,2-trifluoroethyl)benzo[b]thiophene (0.674 g, 1.74 mmol), Iron (0.605 g, 10.83 mmol), NH₄Cl (0.951 g, 17.78 mmol), EtOH (8 mL) and H₂O (1.5 mL). The reaction mixture was stirred at 80° C. for 1 h. The reaction mixture was extracted with EA (100 mL×1), washed with brine (50 mL), dried over anhydrous sodium sulfate, filtered and concentrated under vacuum. This resulted in 0.621 g (99.87%) of 2-iodo-3-(2,2,2-trifluoroethyl)benzo[b]thiophen-7-amine as grey solid. LCMS: m/z=358 [M+1]⁺

### Step 2. tert-butyl (Z)-3-fluoro-4-((2-iodo-3-(2,2,2-trifluoroethyl)benzo[b]thiophen-7-yl)amino)piperidine-1-carboxylate (racemic)

A mixture of 2-iodo-3-(2,2,2-trifluoroethyl)benzo[b]thiophen-7-amine (0.308 g, 0.86 mmol), tert-butyl 3-fluoro-4-oxopiperidine-1-carboxylate (0.455 g, 2.09 mmol), and TMSCl (1.156 g, 10.64 mmol) in DMF (5 mL) was degassed and purged with N₂, and then BH₃·THF (1 M, 9 mL) was added. The mixture was stirred at 0° C. for 1 h under N₂ (g) atmosphere. The reaction mixture was quenched with water (20 mL) at 0° C. and extracted with EA (40 mL×2). The combined organic layers was washed with brine (30 mL), dried over anhydrous sodium sulfate, filtered and concentrated under reduced pressure. The residue was purified by silica gel column, eluted with EA/hexane (v/v=1/5) to afford tert-butyl (Z)-3-fluoro-4-((2-iodo-3-(2,2,2-trifluoroethyl)benzo[b]thiophen-7-yl)amino)piperidine-1-carboxylate (racemic). LCMS: m/z=559 [M+1]⁺

### Step 3. tert-butyl (Z)-3-fluoro-4-((2-(3-((2-methoxy-4-(methylsulfonyl)phenyl)amino)prop-1-yn-1-yl)-3-(2,2,2-trifluoroethyl)benzo[b]thiophen-7-yl)amino)piperidine-1-carboxylate (racemic)

Into a 20-mL sealed tube purged and maintained with an inert atmosphere of nitrogen, was placed tert-butyl (Z)-3-fluoro-4-((2-iodo-3-(2,2,2-trifluoroethyl)benzo[b]thiophen-7-yl)amino)piperidine-1-carboxylate (racemic) (0.306 g, 0.55 mmol), 2-methoxy-4-(methylsulfonyl)-N-(prop-2-yn-1-yl)aniline (0.184 g, 0.77 mmol), Pd(dppf)Cl₂ (0.103 g, 0.15 mmol), CuI (0.082 g, 0.43 mmol), DIEA (0.253 g, 1.96 mmol), DMSO (5 mL). The reaction mixture was stirred at room temperature for 1 h. The reaction mixture was quenched with water (20 mL) and extracted with EA (40 mL×2). The combined organic layers was washed with brine (20 m L), separated and concentrated under vacuum. The residue was purified by silica gel column, eluted with EA/hexane (v/v=3/1) to afford tert-butyl (Z)-3-fluoro-4-((2-(3-((2-methoxy-4-(methylsulfonyl)phenyl)amino)prop-1-yn-1-yl)-3-(2,2,2-trifluoroethyl)benzo[b]thiophen-7-yl)amino)piperidine-1-carboxylate (racemic) (0.338 g, 92.09% yield) as a little yellow solid. LCMS: m/z=670 [M+1]⁺

### Step 4. (Z)-3-fluoro-N-(2-(3-((2-methoxy-4-(methylsulfonyl)phenyl)amino)prop-1-yn-1-yl)-3-(2,2,2-trifluoroethyl)benzo[b]thiophen-7-yl)piperidin-4-amine (racemic)

Into a 100-mL round-bottom flask was placed tert-butyl (Z)-3-fluoro-4-((2-(3-((2-methoxy-4-(methylsulfonyl)phenyl)amino)prop-1-yn-1-yl)-3-(2,2,2-trifluoroethyl)benzo[b]thiophen-7-yl)amino)piperidine-1-carboxylate (racemic) (0.319 g, 0.48 mmol), DCM (5 mL) and TFA (1 mL). the reaction mixture was stirred at room temperature for 1 h. The reaction was adjusted to pH=9 with NaHCO₃ (aq.) and extracted with EA (100 mL×2). The combined organic layers were washed with brine (40 mL), separated and concentrated under vacuum. This resulted in (0.220 g, 81.09%) of (Z)-3-fluoro-N-(2-(3-((2-methoxy-4-(methylsulfonyl)phenyl)amino)prop-1-yn-1-yl)-3-(2,2,2-trifluoroethyl)benzo[b]thiophen-7-yl)piperidin-4-amine (racemic) as yellow oil. LCMS: m/z=570 [M+1]⁺.

### Step 5. (Z)-3-fluoro-N-(2-(3-((2-methoxy-4-(methylsulfonyl)phenyl)amino)prop-1-yn-1-yl)-3-(2,2,2-trifluoroethyl)benzo[b]thiophen-7-yl)-1-methylpiperidin-4-amine (racemic) (2)

To a solution of (Z)-3-fluoro-N-(2-(3-((2-methoxy-4-(methylsulfonyl)phenyl)amino)prop-1-yn-1-yl)-3-(2,2,2-trifluoroethyl)benzo[b]thiophen-7-yl)piperidin-4-amine (racemic) (0.100 g, 0.18 mmol), paraformaldehyde (0.009 g, 0.30 mmol) in 3 mL MeOH. The mixture was stirred at room temperature for 0.5 h. Then NaBH₃CN (0.032 g, 0.75 mmol) and HOAc (0.002 mL) were added. The reaction mixture was stirred at room temperature for 2 days. The residue was purified by preparative HPLC (Mobile Phase A: water (10 mmoL/L ammonium hydroxide), Mobile Phase B: MeOH; Flow rate: 60 mL/min; Gradient: 50-80-100% B (2-30-60 min); 244 nm; RT: 35.560-37.110 min) to afford (Z)-3-fluoro-N-(2-(3-((2-methoxy-4-(methylsulfonyl)phenyl)amino)prop-1-yn-1-yl)-3-(2,2,2-trifluoroethyl)benzo[b]thiophen-7-yl)-1-methylpiperidin-4-amine (racemic) (2) (0.052 g, 50.75% yield) as white solid. LCMS: m/z=584 [M+1]⁺

¹H NMR (400 MHz, MeOD) δ 7.48 (d, J=8.0 Hz, 1H), 7.36-7.25 (m, 2H), 7.19 (d, J=7.6 Hz, 1H), 6.93 (d, J=8.4 Hz, 1H), 6.77 (d, J=7.2 Hz, 1H), 4.77 (s, 1H), 4.40 (s, 2H), 3.95 (s, 3H), 3.78-3.62 (m, 3H), 3.23-3.14 (m, 1H), 3.06 (s, 3H), 2.92 (d, J=11.2 Hz, 1H), 2.39 (d, J=13.2 Hz, 1H), 2.30 (s, 3H), 2.28-2.19 (m, 1H), 1.96 (d, J=10.8 Hz, 2H).

### Example 3

### N-(2-(3-((2-methoxy-4-(methylsulfonyl)phenyl)amino)prop-1-yn-1-yl)-3-(2,2,2-trifluoroethyl)benzo[b]thiophen-7-yl)-1-methylpiperidin-4-amine (3)

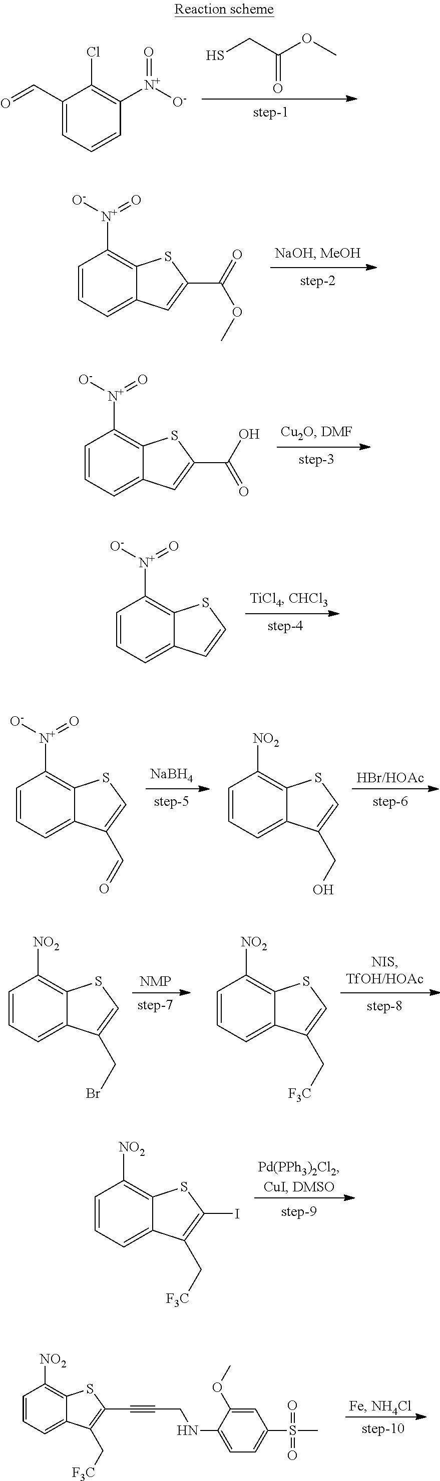

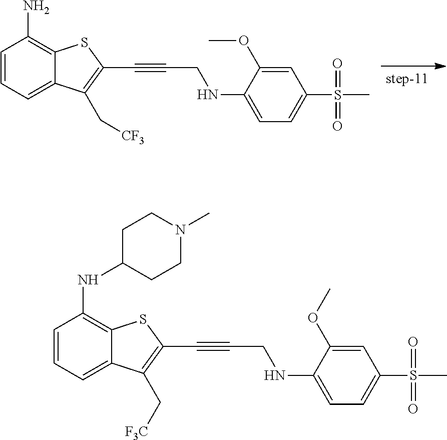 [SMILES: COc1cc(S(C)(=O)=O)ccc1NCC#Cc1sc2c(N)cccc2c1CC(F)(F)F ; COc1cc(S(C)(=O)=O)ccc1NCC#Cc1sc2c(NC3CCN(C)CC3)cccc2c1CC(F)(F)F]

### Experimental Details

### Step 1. methyl 7-nitrobenzo[b]thiophene-2-carboxylate

To a mixture of 2-chloro-3-nitrobenzaldehyde (10.029 g, 0.05 mol) and K₂CO₃ (8.235 g, 0.06 mol) in 100 mL DMF was added methyl 2-mercaptoacetate (6.193 g, 0.06 mol) at 0° C. The mixture was stirred at room temperature for 8 hours and the mixture was added to 500 mL water, the mixture was filtered and washed with water and the filter cake was dried in an oven. This resulted in 11.91 g of methyl 7-nitrobenzo[b]thiophene-2-carboxylate as off-white solid.

### Step 2. 7-nitrobenzo[b]thiophene-2-carboxylic acid

To a 0° C. solution of methyl 7-nitrobenzo[b]thiophene-2-carboxylate (2.44 g, 0.01 mmol) in 20 mL MeOH was added NaOH (2N, 20 mL). The reaction mixture was stirred at room temperature for 8 h. The mixture was concentrated under vacuum. The mixture was adjusted to pH 2-3 with HCl (6 N). The mixture was extracted with EA (100 mL). The organic layers was combined and dried over MgSO₄, filtered and concentrated under vacuum to afford 2.27 g of 7-nitrobenzo[b]thiophene-2-carboxylic acid as little yellow solid.

### Step 3. 7-nitrobenzo[b]thiophene

Into a 40-mL sealed tube was placed 7-nitrobenzo[b]thiophene-2-carboxylic acid (2.105 g, 9.43 mmol), Cu₂O (0.335 g, 1.59 mmol), DMF (20 mL). The reaction mixture was stirred at 120° C. for 12 h. The mixture was added to 100 mL water and a lot of precipitation appeared. The mixture was filtered and the filter cake was washed with water and dried in the oven. This resulted in 1.765 g of 7-nitrobenzo[b]thiophene as grey solid.

### Step 4. 7-nitrobenzo[b]thiophene-3-carbaldehyde

Into a 20-mL sealed tube was placed 7-nitrobenzo[b]thiophene (0.51 g, 2.85 mmol), dichloro(methoxy)methane (1.65 g, 14.35 mmol), TiCl₄ (1.50 g, 7.91 mmol), CHCl₃ (5 mL). The reaction mixture was stirred at 60° C. for 2 h. The reaction was quenched with water (50 mL), extracted with EA (3×100 mL), washed with brine (100 mL), dried over anhydrous sodium sulfate, filtered and concentrated under vacuum. The crude was purified by silica gel column eluted with EA/hexane (v/v=1/3). This resulted in 0.268 g of 7-nitro benzo[b]thiophene-3-carbaldehyde as yellow solid.

### Step 5. (7-nitrobenzo[b]thiophen-3-yl)methanol

To a 0° C. solution of methyl 7-nitrobenzo[b]thiophene-3-carbaldehyde (1.699 g, 8.20 mmol) in 20 mL MeOH was added NaBH₄ (0.656 g, 17.34 mmol). The reaction mixture was stirred at room temperature for 0.5 h. The reaction was quenched with water (50 mL), extracted with EA (3×100 mL), washed with brine (100 mL), dried over anhydrous sodium sulfate, filtered, and concentrated under vacuum. This resulted in 1.197 g of (7-nitrobenzo[b]thiophen-3-yl)methanol as light yellow solid.

### Step 6. 3-(bromomethyl)-7-nitrobenzo[b]thiophene

Into a 20-mL sealed tube was placed (7-nitrobenzo[b]thiophen-3-yl)methanol (0.45 g, 2.15 mmol), 30% HBr/HOAc (5 mL). The reaction mixture was stirred at room temperature for 1 h. The reaction was quenched with water (50 mL), extracted with EA (3×100 mL), washed with brine (100 mL), dried over anhydrous sodium sulfate, filtered and concentrated under vacuum. The crude was purified by silica gel column eluted with EA/hexane (v/v=1/6). This resulted in 0.446 g of 3-(bromomethyl)-7-nitrobenzo[b]thiophene as yellow solid.

### Step 7. 7-nitro-3-(2,2,2-trifluoroethyl)benzo[b]thiophene

Into a 20-mL sealed tube was placed 3-(bromomethyl)-7-nitrobenzo[b]thiophene (0.32 g, 1.18 mmol), copper (0.221 g, 3.48 mmol), diphenyl-(trifluoromethyl)-sulfonium trifluoromethanesulfonate (0.918 g, 2.27 mmol), NMP (3 mL). The reaction mixture was stirred at 60° C. for 1 h. The reaction was quenched with water (40 mL), extracted with EA (2×100 mL), washed with brine (100 mL), dried over anhydrous sodium sulfate, filtered and concentrated under vacuum. The crude was purified by silica gel column eluted with EA/hexane (v/v=1/5). This resulted in 0.295 g of 7-nitro-3-(2,2,2-trifluoroethyl)benzo[b]thiophene as yellow solid.

### Step 8. 2-iodo-7-nitro-3-(2,2,2-trifluoroethyl)benzo[b]thiophene

Into a 20-mL sealed tube was placed 7-nitro-3-(2,2,2-trifluoroethyl)benzo[b]thiophene (0.293 g, 1.12 mmol), NIS (0.336 g, 1.49 mmol), trifluoromethanesulfonic acid (0.5 mL), AcOH (4 mL). The reaction mixture was stirred at room temperature for 3 h. The reaction was quenched with water (30 mL), extracted with EA (2×100 mL), washed with brine (80 mL), dried over anhydrous sodium sulfate, filtered and concentrated under vacuum. The crude was purified by silica gel column eluted with EA/hexane (v/v=1/8). This resulted in 0.389 g of 2-iodo-7-nitro-3-(2,2,2-trifluoroethyl)benzo[b]thiophene as yellow solid.

### Step 9. 2-methoxy-4-(methylsulfonyl)-N-(3-(7-nitro-3-(2,2,2-trifluoroethyl)benzo[b]thiophen-2-yl)prop-2-yn-1-yl)aniline

Into a 20-mL sealed tube purged and maintained with an inert atmosphere of nitrogen, was placed 2-iodo-7-nitro-3-(2,2,2-trifluoroethyl)benzo[b]thiophene (0.380 g, 0.98 mmol), 2-methoxy-4-(methylsulfonyl)-N-(prop-2-yn-1-yl)aniline (0.259 g, 1.08 mmol), Pd(dppf)Cl₂ (0.080 g, 0.11 mmol), CuI (0.053 g, 0.28 mmol), DIE A (0.413 g, 3.20 mmol), DMSO (5 mL). The reaction mixture was stirred at room temperature for 1 h. The reaction mixture was quenched with water (100 mL), extracted with EA (100 mL×2), washed with brine (80 mL), dried over anhydrous sodium sulfate, filtered and concentrated under vacuum. The crude was purified by silica gel column eluted with EA/hexane (v/v=2/3). This resulted in 0.526 g (crude) of 2-m ethoxy-4-(methylsulfonyl)-N-(3-(7-nitro-3-(2,2,2-trifluoroethyl)benzo[b]thiophen-2-yl)prop-2-yn-1-yl)aniline as yellow oil. LCMS: m/z=499 [M+1]⁺

### Step 10. 2-(3-((2-methoxy-4-(methylsulfonyl)phenyl)amino)prop-1-yn-1-yl)-3-(2,2,2-trifluoroethyl)benzo[b]thiophen-7-amine

Into a 20-mL sealed tube was placed 2-methoxy-4-(methylsulfonyl)-N-(3-(7-nitro-3-(2,2,2-trifluoroethyl)benzo[b]thiophen-2-yl)prop-2-yn-1-yl)aniline (0.518 g, 1.04 mmol), iron (0.391 g, 7.00 mmol), NH₄Cl (0.430 g, 8.04 mmol), EtOH (5 mL) and H₂O (1 mL). The reaction mixture was stirred at 80° C. for 1 h. The reaction mixture was extracted with EA (100 mL×1), washed with brine (50 mL), dried over anhydrous sodium sulfate, filtered and concentrated under vacuum. This resulted in 0.282 g (57.92%) of 2-(3-((2-methoxy-4-(methylsulfonyl)phenyl)amino)prop-1-yn-1-yl)-3-(2,2,2-trifluoroethyl)benzo[b]thiophen-7-amine as yellow solid. LCMS: m/z=469 [M+1]⁺

### Step 11. N-(2-(3-((2-methoxy-4-(methylsulfonyl)phenyl)amino)prop-1-yn-1-yl)-3-(2,2,2-trifluoroethyl)benzo[b]thiophen-7-yl)-1-methylpiperidin-4-amine (3)

Into a 25-mL round-bottom flask was placed 2-(3-((2-methoxy-4-(methylsulfonyl)phenyl)amino)prop-1-yn-1-yl)-3-(2,2,2-trifluoroethyl)benzo[b]thiophen-7-amine (0.220 g, 0.43 mmol), 1-methylpiperidin-4-amine (0.259 g, 2.29 mmol), MeOH (4 mL). The mixture was stirred at room temperature for 0.5 h. Then NaBH₃CN (0.177 g, 4.12 mmol) and HOAc (0.02 mL) were added and stirred for 48 h. The mixture was purified by preparative HPLC (Mobile Phase A: water (10 mmoL/L ammonium hydroxide), Mobile Phase B: ACN; Flow rate: 70 mL/min; Gradient: 50-75-100% B (2-30-60 min); 270 nm; RT: 44.886-46.470 min) to afford N-(2-(3-((2-methoxy-4-(methylsulfonyl)phenyl)amino)prop-1-yn-1-yl)-3-(2,2,2-trifluoroethyl)benzo[b]thiophen-7-yl)-1-methylpiperidin-4-amine (3) (0.038 g, 15.58% yield) as off-white solid. LCMS: m/z=566 [M+1]⁺

¹H NMR (400 MHz, MeOD) δ 7.48 (d, J=8.0 Hz, 1H), 7.34-7.21 (m, 2H), 7.12 (d, J=7.2 Hz, 1H), 6.93 (d, J=8.4 Hz, 1H), 6.70 (d, J=7.6 Hz, 1H), 4.40 (s, 2H), 3.95 (s, 3H), 3.74-3.60 (m, 2H), 3.50 (s, 1H), 3.06 (s, 3H), 2.90 (d, J=11.6 Hz, 2H), 2.31 (s, 3H), 2.21 (d, J=11.2 Hz, 2H), 2.06 (d, J=12.0 Hz, 2H), 1.69-1.53 (m, 2H).

### Example 4

### 4-((3-(7-((1-methylpiperidin-4-yl)amino)-3-(2,2,2-trifluoroethyl)benzo[b]thiophen-2-yl)prop-2-yn-1-yl)amino) benzenesulfonamide (4)

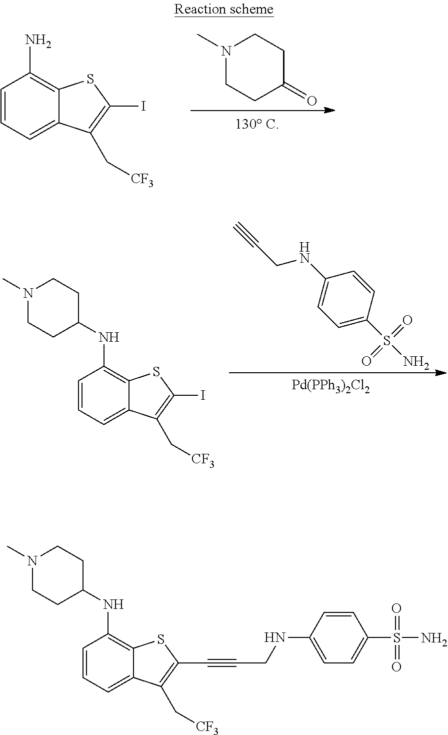

### Experimental Details

### Step 1. N-(2-iodo-3-(2,2,2-trifluoroethyl)benzo[b]thiophen-7-yl)-1-methylpiperidin-4-amine

Into a 4 mL vail was placed 2-iodo-3-(2,2,2-trifluoroethyl)benzo[b]thiophen-7-amine (0.108 g, 302.41 μmol), 1-methylpiperidin-4-one (0.023 g, 203.26 μmol). The vail was heated to 130° C. and stirred for 0.5 h. The reaction was cooled to room temperature, and sodium cyanoborohydride (0.113 g, 2.634 mmol), acetic acid (0.01 μmol), Ethanol (1 mL) was added. The reaction was stirred for 1 h at 50° C. LCMS showed the reaction was complete, the reaction was concentrated under vacuum and purified by C₁₈ column, eluted with ACN/water (v/v=1/3) to afford N-(2-iodo-3-(2,2,2-trifluoroethyl)benzo[b]thiophen-7-yl)-1-methylpiperidin-4-amine (0.068 g, 149.68 μmol, 49.50% yield) as off-white solid. LCMS: m/z=455[M+1]⁺.

### Step 2. 4-((3-(7-((1-methylpiperidin-4-yl)amino)-3-(2,2,2-trifluoroethyl)benzo[b]thiophen-2-yl)prop-2-yn-1-yl) amino)benzenesulfonamide (4)

Into a 4 mL flask purged and maintained with nitrogen atmosphere was placed N-(2-iodo-3-(2,2,2-trifluoroethyl)benzo[b]thiophen-7-yl)-1-methylpiperidin-4-amine (0.067 g, 147.48 μmol), 4-(prop-2-yn-1-ylamino)benzenesulfonamide (0.058 g, 275.86 μmol), Pd(PPh₃)₂Cl₂ (0.011 g, 15.58 μmol), CuI (0.007 g, 36.76 μmol), TEA (0.031 g, 306.36 μmol), DMF (1 mL), and stirred overnight at room temperature. LCMS showed the reaction was complete. The reaction was quenched with water (4 mL), extracted with EA (2 mL×2). The combined organic layers were washed with water (2 mL) and brine (2 mL) successively, separated, then concentrated with vacuum. The residue was purified with prep-HPLC (Mobile Phase A: water (ammonium hydroxide), Mobile Phase B: ACN; Flow rate: 70 mL/min; Gradient: 50-80-100% B (2-30-60 min); 265 nm; R T: 33.245-35.153 min) to afford 4-((3-(7-((1-methylpiperidin-4-yl)amino)-3-(2,2,2-trifluoroethyl)benzo[b]thio phen-2-yl)prop-2-yn-1-yl)amino)benzenesulfonamide (4) (0.021 g, 39.13 μmol, 26.53% yield) as off-white solid. LCMS: m/z=537[M+1]⁺.

¹H NMR (400 MHz, DMSO-d₆) δ 7.58 (d, J=8.4 Hz, 2H), 7.26 (t, J=7.8 Hz, 1H), 7.15 (d, J=7.9 Hz, 1H), 6.98 (s, 2H), 6.89 (t, J=6.1 Hz, 1H), 6.78 (d, J=8.4 Hz, 2H), 6.67 (d, J=7.8 Hz, 1H), 5.31 (d, J=7.9 Hz, 1H), 4.33 (d, J=6.0 Hz, 2H), 3.84 (q, J=11.1 Hz, 2H), 2.77 (d, J=11.0 Hz, 2H), 2.18 (s, 3H), 2.01 (t, J=11.6 Hz, 2H), 1.88 (d, J=11.8 Hz, 2H), 1.63-1.47 (m, 2H).

### Example 5

### 4-((3-(7-(((3R,4S)-3-fluoro-1-methylpiperidin-4-yl)amino)-3-(2,2,2-trifluoroethyl)benzo[b]thiophen-2-yl)prop-2-yn-1-yl)amino)benzenesulfonamide (racemic) (5) and 4-((3-(7-(((3R,4R)-3-fluoro-1-methylpiperidin-4-yl) amino)-3-(2,2,2-trifluoroethyl)benzo[b]thiophen-2-yl)prop-2-yn-1-yl)amino)benzenesulfonamide (racemic) (5b)

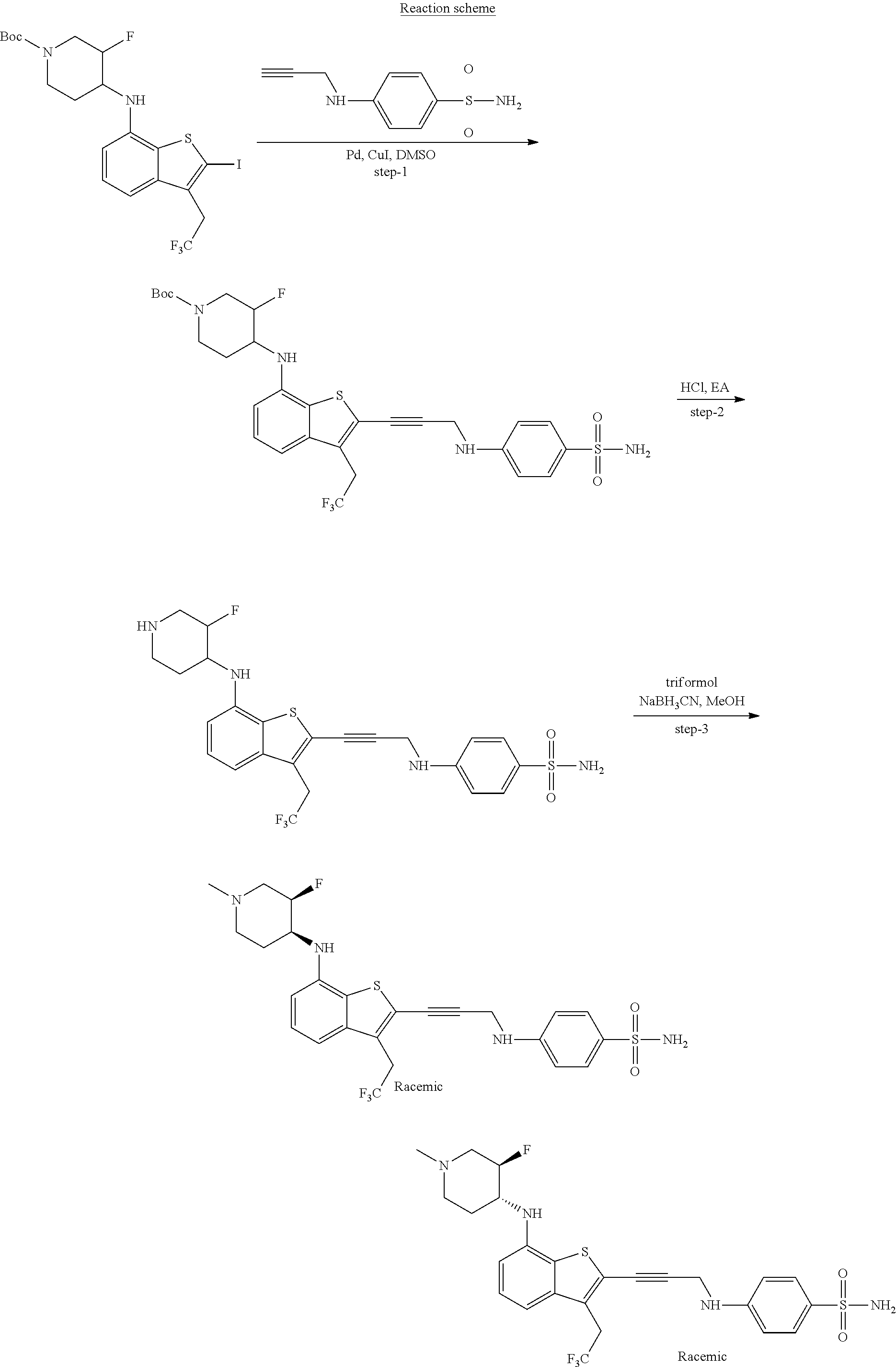

### Experimental Details

### Step 1 tert-butyl-3-fluoro-4-((2-(3-((4-sulfamoylphenyl)amino)prop-1-yn-1-yl)-3-(2,2,2-trifluoroethyl)benzo[b]thiophen-7-yl)amino)piperidine-1-carboxylate

Into a 50 mL 3-necked flask was placed tert-butyl 3-fluoro-4-((2-iodo-3-(2,2,2-trifluoroethyl)benzo[b]thiophen-7-yl)amino)piperidine-1-carboxylate (0.688 g, 1.23 mmol), 4-(prop-2-yn-1-ylamino) benzenesulfonamide (0.561 g, 1.51 mmol), Pd(PPh₃)₂Cl₂ (0.184 g, 260.65 μmol), CuI (0.081 g, 425.30 μmol), DIEA (0.573 g, 4.43 mmol), methyl sulfoxide (7 mL). The reaction was stirred under nitrogen atmosphere at RT for 3 h. The reaction was quenched with water (20 mL). The resulted solution was extracted with EA (3×50 mL), washed with brine (50 mL), dried over anhydrous sodium sulfate, filtered and concentrated under vacuum. The crude was purified by silica gel column eluted with EA/hexane (v/v=3/2). This resulted in 0.642 g (1.00 mmol 81.32% yield) of tert-butyl 3-fluoro-4-((2-(3-((4-sulfamoylphenyl)amino)prop-1-yn-1-yl)-3-(2,2,2-trifluoroethyl)benzo[b]thiophen-7-yl)amino)piperidine-1-carboxylate as yellow solid. LCMS: m/z=641 [M+1]⁺.

### Step 2 4-((3-(7-((3-fluoropiperidin-4-yl)amino)-3-(2,2,2-trifluoroethyl)benzo[b]thiophen-2-yl)prop-2-yn-1-yl) amino)benzenesulfonamide

Into a 20 mL sealed tube was placed tert-butyl 3-fluoro-4-((2-(3-((4-sulfamoylphenyl)amino)prop-1-yn-1-yl)-3-(2,2,2-trifluoroethyl)benzo[b]thiophen-7-yl)amino)piperidine-1-carboxylate (0.622 g, 970.79 mmol), HCl(g) in EA (2 mL, 4N), EA (2 mL). The reaction was stirred at RT for 1 h. The reaction was concentrated under vacuum, water (2 mL) was added to the reaction. NaOH (aq, 3N) was added until PH=7, extracted with EA (3×50 mL), washed with brine (30 mL), dried over anhydrous sodium sulfate, filtered and concentrated under vacuum. This resulted in 0.530 g (980.40 μmol, 100.00% yield) of 4-((3-(7-((3-fluoropiperidin-4-yl)amino)-3-(2,2,2-trifluoroethyl)benzo[b]thiophen-2-yl)prop-2-yn-1-yl)amino)benzenesulfonamide as brown solid. LCMS: m/z=274[M+1]⁺. The reaction mixture was purified by C18 column eluted with ACN/H₂O (v/v=1/1). This resulted in 3.012 g (74% yield) of ethyl N-(3-bromo-5-nitro-4-(1H-pyrrol-1-yl)phenyl)-1-meth ylpiperidin-4-amine as yellow solid. LCMS: m/z=541 [M+1]⁺.

### Step 3 4-((3-(7-(((3R,4S)-3-fluoro-1-methylpiperidin-4-yl)amino)-3-(2,2,2-trifluoroethyl)benzo[b]thiophen-2-yl)prop-2-yn-1-yl)amino)benzenesulfonamide (racemic) (5) and 4-((3-(7-(((3R,4R)-3-fluoro-1-methylpiperidin-4-yl)amino)-3-(2,2,2-trifluoroethyl)benzo[b]thiophen-2-yl)prop-2-yn-1-yl)amino)benzenesulfonamide (race mic) (5b)

Into a 25-mL round-bottom flask was placed 4-((3-(7-((3-fluoropiperidin-4-yl)amino)-3-(2,2,2-trifluoroethyl)benzo[b]thiophen-2-yl)prop-2-yn-1-yl)amino)benzenesulfonamide (0.317 g, 586.38 umol), triformol (0.043 g, 1.43 mmol), acetic acid (0.5 mL), methyl alcohol (5 mL) The reaction mixture was stirred at RT for 1 h. Then NaBH₃CN (0.231 g, 5.38 mmol) was added. The reaction mixture was stirred at RT for 1 h. The reaction was quenched with water (20 mL). The resulted solution was extracted with EA (3×50 mL), washed with brine (50 mL), dried over anhydrous sodium sulfate, filtered and concentrated under vacuum. The crude product was purified by preparative HPLC (Mobile Phase A: water (0.1% ammonium hydroxide), Mobile Phase B: ACN; Flow rate: 70 mL/min; Gradient: 35-65-75% B (2-30-60 min); 264 nm; RT: 30.01-31.74) to provide the desired product. This resulted in 0.055 g (99.16 μmol, 16.91% yield) of 4-((3-(7-(((3R,4S)-3-fluoro-1-methylpiperidin-4-yl)amino)-3-(2,2,2-trifluoroethyl)benzo[b]thiophen-2-yl)prop-2-yn-1-yl)amino)benzene sulfonamide (racemic) (5) as white solid. LCMS: m/z=555 [M+1]⁺.

¹H NMR (400 MHz, DMSO-d₆) δ 7.58 (d, J=8.4 Hz, 2H), 7.31-7.20 (m, 2H), 6.97 (s, 2H), 6.98-6.92 (m, 1H), 6.78 (d, J=8.2 Hz, 3H), 5.17 (d, J=8.5 Hz, 1H), 4.79 (d, J=49.5 Hz, 1H), 4.33 (d, J=6.0 Hz, 2H), 3.88-3.72 (m, 2H), 3.82-3.61 (m, 1H) 3.03 (t, J=11.1 Hz, 1H), 2.79 (d, J=11.1 Hz, 1H), 2.27 (d, J=13.0 Hz, 1H), 2.18 (s, 3H), 2.08 (t, J=11.4 Hz, 1H), 2.01-1.89 (m, 1H), 1.71 (d, J=11.9 Hz, 1H).

And 0.027 g (48.68 μmol, 8.30% yield) of 4-((3-(7-(((3R,4R)-3-fluoro-1-methylpiperidin-4-yl)amino)-3-(2,2,2-trifluoroethyl)benzo[b]thiophen-2-yl)prop-2-yn-1-yl)amino)benzenesulfonamide (racemic) (5b) as white solid. LCMS: m/z=555 [M+1]⁺.

¹H NMR (400 MHz, DMSO-d₆) δ 7.58 (d, J=8.4 Hz, 2H), 7.25 (t, J=7.9 Hz, 1H), 7.16 (d, J=7.9 Hz, 1H), 6.98 (s, 2H), 6.90 (t, J=6.1 Hz, 1H), 6.77-6.68 (m, 3H), 5.57 (d, J=8.5 Hz, 1H), 4.71-4.52 (m, 1H), 4.33 (d, J=6.0 Hz, 2H), 3.85-3.79 (m, 2H), 3.59 (d, J=4.2 Hz, 1H), 3.10-3.01 (m, 1H), 2.69 (d, J=11.5 Hz, 1H), 2.23 (s, 3H), 2.04-3.01 (m, 1H), 1.99-1.88 (m, 1H), 1.59-1.47 (m, 1H).

### Example 6

### 1-((Z)-3-fluoro-4-((2-(3-((2-methoxy-4-(methylsulfonyl)phenyl)amino)prop-1-yn-1-yl)-3-(2,2,2-trifluoroethyl)benzo[b]thiophen-7-yl)amino)piperidin-1-yl)-3-methoxypropan-2-ol (racemic) (6)

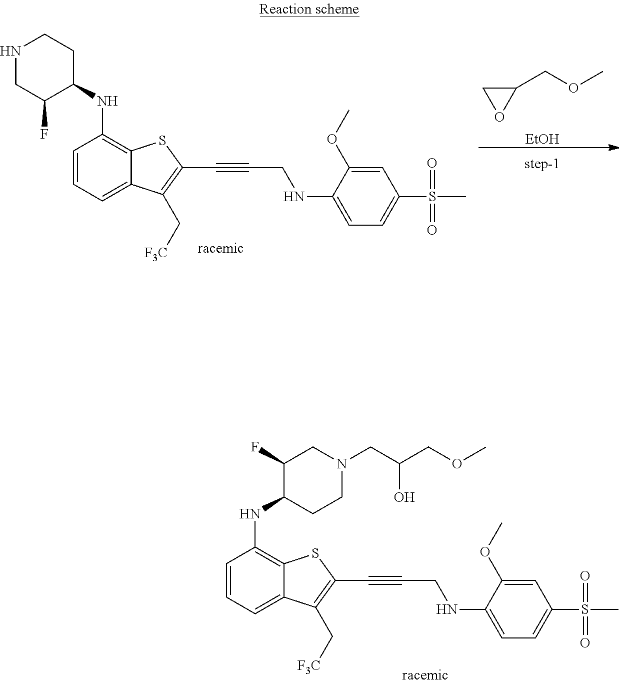

### Experimental Details

### Step 1. 1-((Z)-3-fluoro-4-((2-(3-((2-methoxy-4-(methylsulfonyl)phenyl)amino)prop-1-yn-1-yl)-3-(2,2,2-trifluoroethyl)benzo[b]thiophen-7-yl)amino)piperidin-1-yl)-3-methoxypropan-2-ol (racemic) (6)

Into a 4 mL vial placed (Z)-3-fluoro-N-(2-(3-((2-methoxy-4-(methylsulfonyl)phenyl)amino)prop-1-yn-1-yl)-3-(2,2,2-trifluoroethyl)benzo[b]thiophen-7-yl)piperidin-4-amine (racemic) (0.036 g, 63.20 μmol), 2-(methoxymethyl) oxirane (0.044 g, 499.41 μmol), ethanol (1 mL). The reaction was stirred at RT for 4 h. The reaction was concentrated under vacuum. The mixture was purified by preparative HPLC (Mobile Phase A: water (10 mmol/L ammonium hydroxide), Mobile Phase B: MeOH; Flow rate: 70 mL/min; Gradient: 50-80-100% B (2-30-60 min); 269 nm; RT: 33.838-34.618) to provide the desired product. This resulted in 0.029 g (69.77% yield) of 1-((Z)-3-fluoro-4-((2-(3-((2-methoxy-4-(methylsulfonyl)phenyl)amino)prop-1-yn-1-yl)-3-(2,2,2-trifluoroethyl)benzo[b]thiophen-7-yl)amino)piperidin-1-yl)-3-methoxypropan-2-ol (racemic) (6) as off-white solid. LCMS: m/z=659 [M+1]⁺.

¹H NMR (400 MHz, MeOD) δ 7.49 (d, J=8.3 Hz, 1H), 7.32-7.25 (m, 2H), 7.19 (d, J=8.2 Hz, 1H), 6.94 (d, J=8.4 Hz, 1H), 6.77 (d, J=7.6 Hz, 1H), 4.40 (s, 2H), 3.95 (s, 3H), 3.90 (s, 1H), 3.79-3.61 (m, 3H), 3.45-3.33 (m, 5H), 3.30-3.29 (m, 2H), 3.06 (s, 3H), 2.99 (s, 1H), 2.57-2.44 (m, 2H), 2.44-2.28 (m, 2H), 2.03-1.85 (m, 2H).

### Example 7

### N-(2-(3-((5-fluoro-2-methoxy-4-(methylsulfonyl)phenyl)amino)prop-1-yn-1-yl)-3-(2,2,2-trifluoroethyl)benzo[b]thiophen-7-yl)-1-methylpiperidin-4-amine (7)

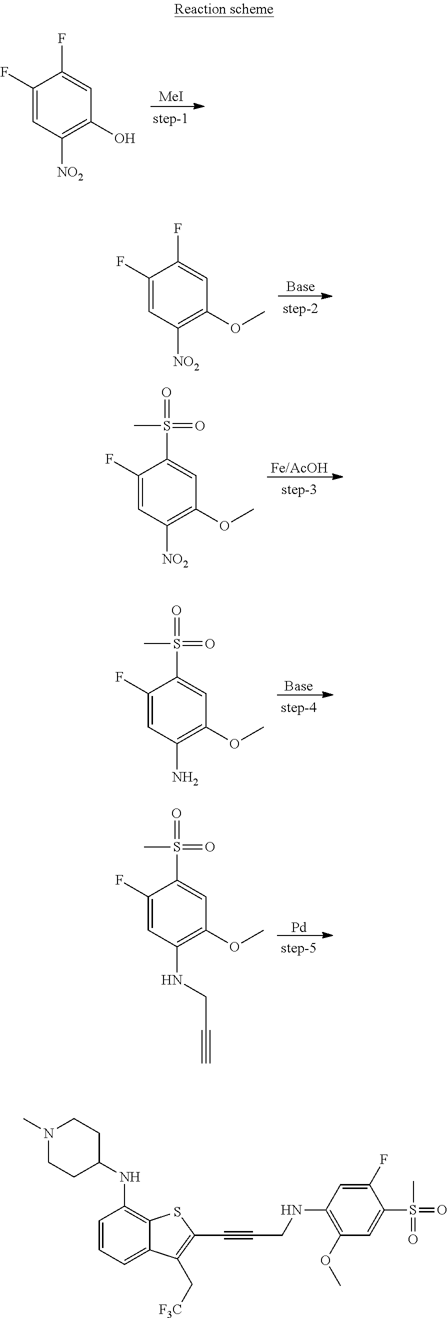

### Experimental Details

### Step 1. 1,2-difluoro-4-methoxy-5-nitrobenzene

Into a 100 mL flask was placed 4,5-difluoro-2-nitrophenol (4.76 g, 27.19 mmol), K₂CO₃ (11.33 g, 81.98 m mol), iodomethane (3 mL), DMF (30 mL). The reaction was stirred at 20° C. for 1 h. The reaction was quenched by water (50 mL), extracted by EA (3×50 mL). The organic layers combined and concentrated under vacuum. The residue was purified by silica gel column eluted with EA/Hex (v/v=2/8). This resulted in 5.30 g (99% yield) of 1,2-difluoro-4-methoxy-5-nitrobenzene as white solid. LCMS: m/z=190 [M+1]⁺.

### Step 2. 1-fluoro-4-methoxy-2-(methylsulfonyl)-5-nitrobenzene

Into a 100 mL flask was placed methyl 1,2-difluoro-4-methoxy-5-nitrobenzene (2.99 g, 15.84 mmol), sodium methanesulfinate (1.90 g, 18.62 mmol), DMA (10 mL). The reaction was stirred at 85° C. for 16 h. The reaction was quenched by water (50 mL). Then a large amount of solids precipitated. The mixture was filtered and collected the filter cake. The filter cake was drying at 60° C. for 16 h. This resulted in 3.00 g (76% yield) of 1-fluoro-4-methoxy-2-(methylsulfonyl)-5-nitrobenzene as white solid. LCMS: m/z=250 [M+1]⁺.

### Step 3. 5-fluoro-2-methoxy-4-(methylsulfonyl)aniline

Into a 100-mL round-bottom flask was placed 1-fluoro-4-methoxy-2-(methylsulfonyl)-5-nitrobenzene (4.62 g, 18.54 mmol), Pd/C (3.47 g, 32.61 mmol), MeOH (50 mL). The flask was charged with H₂. The reaction mixture was stirred at 20° C. for 16 h. The mixture was filtered and collected the filtrate. The filtrate was concentrated under vacuum. The residue was purified by silica gel column eluted with EA/hexane (v/v=2/8). This resulted in 2.27 g (55% yield) of 5-fluoro-2-methoxy-4-(methylsulfonyl)aniline as yellow solid. LCMS: m/z=220[M+1]⁺.

### Step 4. 5-fluoro-2-methoxy-4-(methylsulfonyl)-N-(prop-2-yn-1-yl)aniline

Into a 50-mL round-bottom flask was placed 5-fluoro-2-methoxy-4-(methylsulfonyl)aniline (0.91 g, 4.13 mmol), 3-bromoprop-1-yne (0.62 g, 5.24 mmol), K₂CO₃ (1.86 g, 13.42 mmol), NaI (0.10 g, 0.67 mmol), DMF (20 mL). The mixture was stirred at 85° C. for 16 h. The reaction mixture was purified by C18 column eluted with ACN/H₂O (v/v=1/1). This resulted in 318 mg (29% yield) of 5-fluoro-2-methoxy-4-(methylsulfonyl)-N-(prop-2-yn-1-yl)aniline as light yellow solid. LCMS: m/z=258 [M+1]⁺.

### Step 5. N-(2-(3-((5-fluoro-2-methoxy-4-(methylsulfonyl)phenyl)amino)prop-1-yn-1-yl)-3-(2,2,2-trifluoroethyl) benzo[b]thiophen-7-yl)-1-methylpiperidin-4-amine (7)

Into a 50-mL round-bottom flask was placed N-(2-iodo-3-(2,2,2-trifluoroethyl)benzo[b]thiophen-7-yl)-1-methyl piperidin-4-amine (0.066 g, 145.28 μmol), 5-fluoro-2-methoxy-4-(methylsulfonyl)-N-(prop-2-yn-1-yl)aniline (0.063 g, 244.87 μmol), CuI (0.043 g, 225.78 μmol), Pd(PPh₃)₂Cl₂ (0.051 g, 56.26 μmol), DIEA (0.041 g, 317.23 μmol) and methyl sulfoxide (2 mL). The mixture was stirred at 25° C. for 4 h. The mixture was purified by preparative HPLC (Mobile Phase A: water (10 mmoL/L ammonium hydroxide), Mobile Phase B: A CN; Flow rate: 40 mL/min; Gradient: 40-80-100% B (2-30-60 min); 220 nm; RT: 23.767-27.715 min). This resulted in 22 mg (25% yield) of N-(2-(3-((5-fluoro-2-methoxy-4-(methylsulfonyl)phenyl)amino)prop-1-yn-1-yl)-3-(2,2,2-trifluoroethyl)benzo[b]thiophen-7-yl)-1-methylpiperidin-4-amine (7) as white solid. LCMS: m/z=584 [M+1]⁺.

¹H NMR (400 MHz, DMSO) δ 7.26 (t, J=7.9 Hz, 1H), 7.15 (d, J=8.0 Hz, 1H), 7.06 (d, J=6.4 Hz, 1H), 6.88 (t, J=5.7 Hz, 1H), 6.77 (d, J=12.6 Hz, 1H), 6.67 (d, J=7.8 Hz, 1H), 5.33 (d, J=7.9 Hz, 1H), 4.39 (d, J=6.2 Hz, 2H), 3.34 (s, 5H), 3.18 (s, 3H), 2.76 (d, J=11.2 Hz, 2H), 2.17 (s, 3H), 2.00 (t, J=10.9 Hz, 2H), 1.89 (d, J=14.3 Hz, 2H), 1.63-1.46 (m, 2H), 1.23 (s, 1H).

### Example 8

### 3-methoxy-N-methyl-4-((3-(7-((1-methylpiperidin-4-yl)amino)-3-(2,2,2-trifluoroethyl)benzo[b]thiophen-2-yl)prop-2-yn-1-yl)amino)benzamide (8)

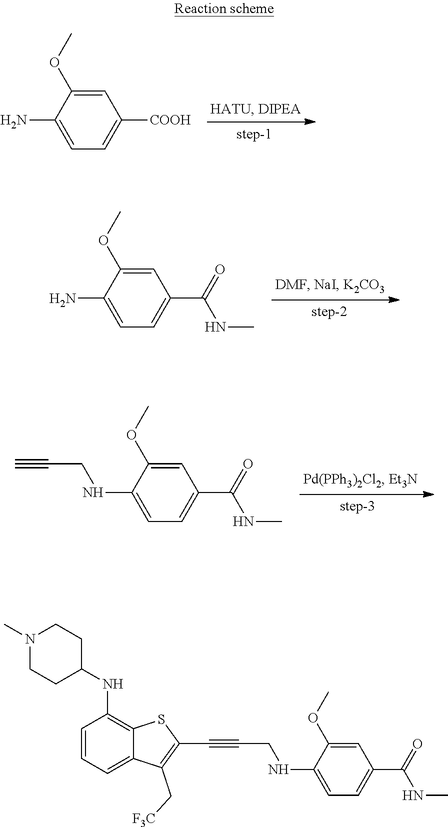

### Experimental Details

### Step 1. 4-amino-3-methoxy-N-methylbenzamide

Into a 1 L flask was placed 4-amino-3-methoxybenzoic acid (10.01 g, 59.88 mmol), 2-(7-aza-1H-benzotriazole-1-yl)-1,1,3,3-Tetramethyluronium hexafluorophosphate (68.52 g, 180.2070 mmol), N,N-diisopropylethylamine (20.96 g, 162.18 mmol), methylamine hydrochloride (24.87 g, 368.35 mmol), N,N-dimethylformamide (500 mL). The reaction mixture was stirred at room temperature for 24 h. The reaction was quenched by the addition of water (10 mL). Then saturated sodium hydroxide aqueous solution was added to the mixture until pH=10˜11. The resulting solution extracted with EA (2×200 mL). The organic layer was combined, washed with brine (200 mL), dried over anhydrous Na₂SO₄ and concentrated under vacuum. The residue was applied onto a silica gel column eluted with EA/heptane (v/v=9/1). This resulted in 8.53 g (79.05% yield) of 4-amino-3-methoxy-N-methylbenzamide as yellow oil. LCMS: m/z=181 [M+1]⁺.

### Step 2. 3-methoxy-N-methyl-4-(prop-2-yn-1-ylamino)benzamide

Into a 500 mL flask purged and maintained with an inert atmosphere of nitrogen, was placed 4-amino-3-m ethoxy-N-methylbenzamide (5.003 g, 27.76 mmol), potassium carbonate (11.775 g, 85.20 mmol), sodium iodide (4.402 g, 29.37 mmol), N,N-dimethylformamide (200 mL). The reaction mixture was stirred 85° C. for 7 h. The residue was applied onto a silica gel column eluted with EA/heptane (v/v=3/1). The reaction w as quenched by the addition of water (200 mL). The resulting solution extracted with EA (2×200 mL). The organic layers was combined, washed with brine (200 mL), dried over anhydrous Na₂SO₄ and concentrated under vacuum. The residue was applied onto a silica gel column eluted with EA/heptane (v/v=2/5). This resulted in 2.686 g (44.33% yield) of 3-methoxy-N-methyl-4-(prop-2-yn-1-ylamino) benzamide as yell ow solid. LCMS: m/z=219 [M+1]⁺.

### Step 3. 3-methoxy-N-methyl-4-((3-(7-((1-methylpiperidin-4-yl)amino)-3-(2,2,2-trifluoroethyl)benzo[b]thiophen-2-yl)prop-2-yn-1-yl)amino)benzamide (8)

Into a 500 mL flask purged and maintained with an inert atmosphere of nitrogen, was placed 3-methoxy-N-methyl-4-(prop-2-yn-1-ylamino)benzamide (0.029 g, 132.87 μmol), N-(2-iodo-3-(2,2,2-trifluoroethyl)benzo[b]thiophen-7-yl)-1-methylpiperidin-4-amine (0.054 g, 118.87 μmol), bis(triphenylphosphine)palladium(II) chloride (0.014 g, 19.83 μmol), cuprous iodide (0.008 g, 42.01 μmol), triethylamine (0.022 g, 217.41 μmol), meth yl sulfoxide (2 mL). The reaction mixture was stirred at room temperature for 2 h. The reaction was quenched by the addition of water (2 mL) and extracted with EA (2×4 mL). The organic layers was combined, washed with brine (5 mL) and concentrated under vacuum. The mixture was purified by preparative H PLC (Mobile Phase A: water (10 mmoL/L ammonium hydroxide), Mobile Phase B: MeOH; Flow rate: 40 mL/min; Gradient: 45-75-100% B (2-30-60 min); 270 nm; RT: 33.580-36.570 min). This resulted in 0.010 g (7.42% yield) of 3-methoxy-N-methyl-4-((3-(7-((1-methylpiperidin-4-yl)amino)-3-(2,2,2-trifluoroethyl)benzo[b]thiophen-2-yl)prop-2-yn-1-yl)amino)benzamide (8) as white solid. LCMS: m/z=545 [M+1]⁺.

¹H NMR (400 MHz, DMSO) δ 8.10 (s, 1H), 7.42 (d, J=8.7 Hz, 1H), 7.35 (s, 1H), 7.23 (d, J=7.8 Hz, 1H), 7.13 (d, J=7.6 Hz, 1H), 6.75 (d, J=8.4 Hz, 1H), 6.65 (d, J=8.1 Hz, 1H), 6.01 (s, 1H), 5.29 (d, J=7.9 Hz, 1H), 4.33 (d, J=6.6 Hz, 2H), 3.84 (s, 3H), 3.81-3.74 (m, 2H), 2.75 (d, J=4.4 Hz, 4H), 2.16 (s, 3H), 1.98 (t, J=11.3 Hz, 2H), 1.87 (d, J=12.1 Hz, 2H), 1.53 (d, J=11.9 Hz, 3H), 1.23 (s, 1H).

### Example 9

dimethyl(4-((3-(7-((1-methylpiperidin-4-yl)amino)-3-(2,2,2-trifluoroethyl)benzo[b]thiophen-2-yl)prop-2-yn-1-yl)amino)phenyl)phosphine oxide (9)

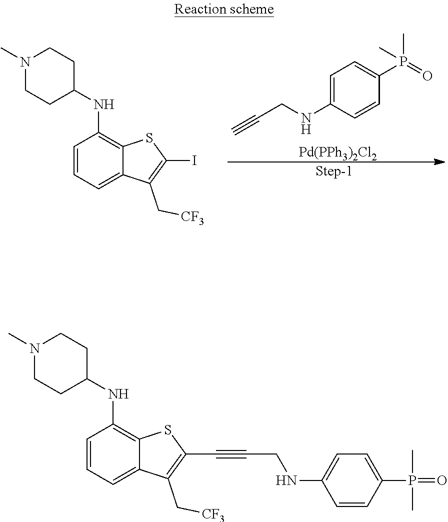 [SMILES: CN1CCC(Nc2cccc3c(CC(F)(F)F)c(I)sc23)CC1 ; C#CCNc1ccc(P(C)(C)=O)cc1 ; CN1CCC(Nc2cccc3c(CC(F)(F)F)c(C#CCNc4ccc(P(C)(C)=O)cc4)sc23)CC1 ; "Pd(PPh3)2Cl2"]

### Experimental Details

### Step 1. dimethyl(4-((3-(7-((1-methylpiperidin-4-yl)amino)-3-(2,2,2-trifluoroethyl)benzo[b]thiophen-2-yl)prop-2-yn-1-yl)amino)phenyl)phosphine oxide (9)

Into a 4 mL flask purged and maintained with nitrogen atmosphere was placed N-(2-iodo-3-(2,2,2-trifluoroethyl)benzo[b]thiophen-7-yl)-1-methylpiperidin-4-amine (0.049 g, 107.86 μmol), dimethyl(4-(prop-2-yn-1-ylamino)phenyl)phosphine oxide (0.068 g, 328.17 μmol), Pd(PPh₃)₂Cl₂ (0.009 g, 12.75 μmol), CuI (0.004 g, 21.00 μmol), TEA (0.015 g, 148.24 μmol), DMF (0.5 mL), and stirred for 1 h at room temperature. LCMS showed the reaction was complete. The reaction was quenched with water (4 mL), extracted with EA (2 mL×2). The combined organic layers were washed with water (2 mL) and brine (2 mL) successively, separated, then concentrated with vacuum. The residue was purified with prep-HPLC (Mobile Phase A: water (trifluoroacetic acid), Mobile Phase B: ACN; Flow rate: 70 mL/min; Gradient: 25-45-45% B (2-30-60 min); 262 n m; RT: 27.501-29.585 min) to afford dimethyl(4-((3-(7-((1-methylpiperidin-4-yl)amino)-3-(2,2,2-trifluoroethyl)benzo[b]thiophen-2-yl)prop-2-yn-1-yl)amino)phenyl)phosphine oxide (9) (0.010 g, 18.74 μmol, 17.38% yield) as off-white solid. LCMS: m/z=534[M+1]⁺.

¹H NMR (400 MHz, DMSO-d₆) δ 7.53-7.46 (m, 2H), 7.25 (t, J=7.9 Hz, 1H), 7.14 (d, J=7.9 Hz, 1H), 6.83-6.78 (m, 2H), 6.72-6.64 (m, 2H), 5.31 (d, J=7.9 Hz, 1H), 4.31 (d, J=6.1 Hz, 2H), 3.80 (q, J=10.8 Hz, 2H), 2.81-2.73 (m, 2H), 2.17 (s, 3H), 2.04-1.95 (m, 2H), 1.91-1.85 (m, 2H), 1.55 (d, J=13.1 Hz, 8H).

### Example 10

### (4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-(fluoromethoxy)phenyl)dimethylphosphine oxide (racemic) (10)

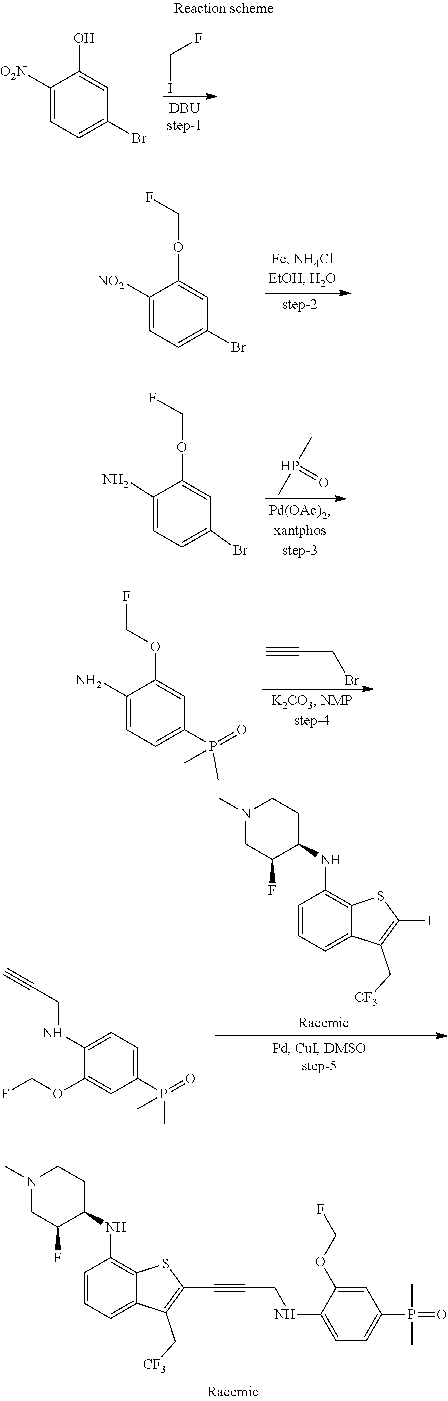

### Experimental Details

### Step 1 4-bromo-2-(fluoromethoxy)-1-nitrobenzene

Into a 50 mL 3-necked flask was placed 5-bromo-2-nitrophenol (1.16 g, 5.32 mmol), fluoroiodomethane (1.43 g, 8.94 mmol), DBU (2.28 g, 14.97 mmol), ACN (10 mL). The reaction was stirred at 80° C. for 1 h. The reaction was quenched with water (10 mL). The resulted solution was extracted with EA (3×30 m L), washed with brine (30 mL), dried over anhydrous sodium sulfate, filtered and concentrated under vacuum. This resulted in 1.276 g (5.10 mmol, 95.91% yield) of 4-bromo-2-(fluoromethoxy)-1-nitrobenzene as yellow solid. LCMS: m/z=250 [M+1]⁺.

### Step 2 4-bromo-2-(fluoromethoxy)aniline

Into a 100 mL 3-necked flask was placed 4-bromo-2-(fluoromethoxy)-1-nitrobenzene (1.332 g, 5.32 mmol), iron (2.678 g, 47.95 mmol), NH₄Cl (2.610 g, 48.79 mmol), EtOH (15 mL), water (3 mL). The reaction w as stirred at 95° C. for 2 h. The reaction mixture was filtered and the filter cake was washed with methanol (2×30 mL). The filterate was concentrated under vacuum to afford the crude product. The crude product was purified by silica gel column eluted with EA/hexane (v/v=1/3). This resulted in 1.01 g (4.59 mmol, 86.15% yield) of 4-bromo-2-(fluoromethoxy)aniline as yellow oil. LCMS: m/z=220 [M+1]⁺.

### Step 3 (4-amino-3-(fluoromethoxy)phenyl)dimethylphosphine oxide

Into a 25 mL 3-necked flask purged and maintained with an inert atmosphere of nitrogen was placed 4-bromo-2-(fluoromethoxy)aniline (0.551 g, 2.50 mmol), Palladium (II) acetate (0.132 g, 587.95 umol), DIEA (0.865 g, 6.69 mmol), DMF (5 mL). The reaction was stirred under nitrogen atmosphere at 130° C. for 0.5 h. Then dimethylphosphine oxide (0.603 g, 7.72 mmol) was added at 130° C. The reaction was stirred un der nitrogen atmosphere at 130° C. for 1 h The reaction mixture was purified by C18 column eluted with ACN/H₂O (v/v=1/9). This resulted in 0.400 g, (1.84 mmol, 73.55% yield) of (4-amino-3-(fluoromethoxy) phenyl)dimethylphosphine oxide as brown oil. LCMS: m/z=218 [M+1]⁺.

### Step 4 (3-(fluoromethoxy)-4-(prop-2-yn-1-ylamino)phenyl)dimethylphosphine oxide

Into a 25 mL 3-necked flask was placed (4-amino-3-(fluoromethoxy)phenyl)dimethylphosphine oxide (0.366 g, 1.68 mmol), 3-bromoprop-1-yne (0.295 g, 2.47 mmol), K₂CO₃ (0.695 g, 5.02 mmol), KI (0.384 g, 2.31 mmol), NMP (5 mL). The reaction mixture was stirred under nitrogen atmosphere at 80° C. for 4 h. The reaction mixture was purified by C18 column eluted with ACN/H₂O (v/v=2/8). This resulted in 0.066 g (258.59 μmol, 15.34% yield) of (3-(fluoromethoxy)-4-(prop-2-yn-1-ylamino)phenyl)dimethylphosphine oxide as brown oil. LCMS: m/z=256 [M+1]⁺.

### Step 5 (4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-(fluoromethoxy)phenyl)dimethylphosphine oxide (racemic) (10)

Into a 10-mL round-bottom flask purged and maintained with an inert atmosphere of nitrogen was placed (3-(fluoromethoxy)-4-(prop-2-yn-1-ylamino)phenyl)dimethylphosphine oxide (0.071 g, 278.15 μmol), (Z)-3-fluoro-N-(2-iodo-3-(2,2,2-trifluoroethyl)benzo[b]thiophen-7-yl)-1-methylpiperidin-4-amine (racemic) (0.051 g, 107.98 μmol, Pd(PPh₃)₂Cl₂ (0.030 g, 42.49 μmol), CuI (0.018 g, 94.51 μmol), DIEA (0.063 g, 487.45 μmol), methyl sulfoxide (1 mL). The reaction was stirred under nitrogen atmosphere at RT for 16 h. The crude product was purified by preparative HPLC (Mobile Phase A: water (0.1% ammonium hydroxide), Mobile Phase B: MeOH; Flow rate: 70 mL/min; Gradient: 30-30-60-80% B (0-2-30-60 min); 262 nm; RT: 33.31-35.22) to provide the desired product. This resulted in 1.05 mg (1.75 μmol, 0.62% yield) of (4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-(fluoro methoxy)phenyl)dimethylphosphine oxide (racemic) (10) as white solid. LCMS: m/z=600 [M+1]⁺.

¹H NMR (400 MHz, DMSO-d₆) δ7.53-7.33 (m, 2H), 7.31-7.11 (m, 2H), 6.96 (s, 1H), 6.79 (s, 1H), 6.33 (s, 1H), 5.92 (s, 1H), 5.78 (s, 1H), 5.16 (s, 1H), 4.80 (d, J=49.3 Hz, 1H), 4.37 (s, 2H), 3.81 (d, J=11.4 Hz, 2H), 3.70-3.65 (m, 1H), 3.10-2.98 (m, 1H), 2.87-2.76 (m, 1H), 2.19 (s, 3H), 2.02-1.93 (m, 2H), 1.76-1.69 (m, 1H), 1.58 (d, J=13.2 Hz, 6H), 1.50-1.41 (m, 1H).

### Example 11

### N-(2-(3-((2-(fluoromethoxy)-4-(methylsulfonyl)phenyl)amino)prop-1-yn-1-yl)-3-(2,2,2-trifluoroethyl)benzo[b]thiophen-7-yl)-1-methylpiperidin-4-amine (11)

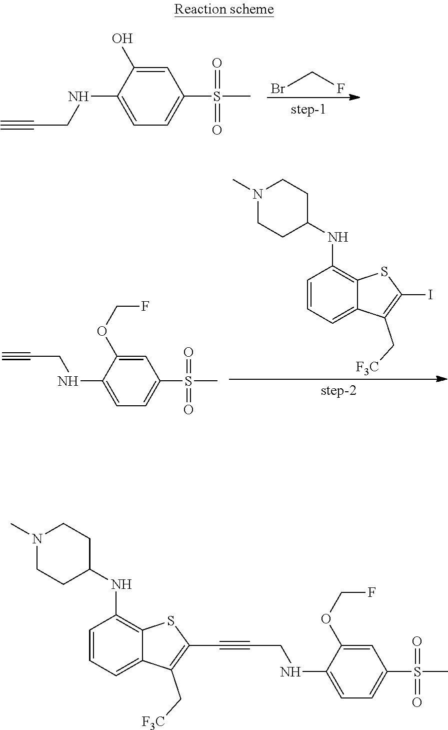

### Experimental Details

### Step 1. 2-(fluoromethoxy)-4-(methylsulfonyl)-N-(prop-2-yn-1-yl)aniline

Into a 8 mL flask was placed 5-(methylsulfonyl)-2-(prop-2-yn-1-ylamino)phenol (0.101 g, 448.36 μmol), bromofluoromethane (0.053 g, 469.32 μmol), potassium carbonate (0.126 g, 911.69 μmol), N,N-dimethylformamide (2 mL). The reaction mixture was stirred at 45° C. for 3 h. The reaction was quenched by the addition of water (2 mL) and extracted with EA (2×5 mL). The organic layer was combined, washed with brine (10 mL), dried over anhydrous Na₂SO₄ and concentrated under vacuum. The residue was applied onto a silica gel column eluted with EA/heptane (v/v=1/1). This resulted in 0.096 g (83.22% yield) of 2-(fluoromethoxy)-4-(methylsulfonyl)-N-(prop-2-yn-1-yl)aniline as yellow oil. LCMS: m/z=258 [M+1]⁺.

### Step 2. N-(2-(3-((2-(fluoromethoxy)-4-(methylsulfonyfol)phenyl)amino)prop-1-yn-1-yl)-3-(2,2,2-trifluoroethyl)benzo[b]thiophen-7-yl)-1-methylpiperidin-4-amine (11)

Into a 8 mL flask purged and maintained with an inert atmosphere of nitrogen, was placed N-(2-iodo-3-(2,2,2-trifluoroethyl)benzo[b]thiophen-7-yl)-1-methylpiperidin-4-Amine (0.051 g, 112.26 μmol), cuprous iodide (0.007 g, 36.76 μmol), 2-(fluoromethoxy)-4-(methylsulfonyl)-N-(prop-2-yn-1-yl)aniline (0.028 g, 108.83 μmol), bis(triphenylphosphine)palladium(II) chloride (0.023 g, 32.58 μmol), triethylamine (0.024 g, 237.18 μmol), methyl sulfoxide (2 mL). The reaction mixture was stirred at room temperature for 2 h. he reaction was quenched by the addition of water (2 mL) and extracted with EA (2×4 mL). The organic layer was combined, washed with brine (5 mL), dried over anhydrous Na₂S₄ and concentrated under vacuum. The mixture was purified by preparative HPLC (Mobile Phase A: water (10 mmoL/L ammonium hydroxide), Mobile Phase B: MeOH; Flow rate: 40 mL/min; Gradient: 50-75-100B (2-30-60 min); 270 nm; RT: 32.200-34.270 min). This resulted in 0.023 g (35.10% yield) of N-(2-(3-((2-(fluoromethoxy)-4-(methylsulfonyl)phenyl) amino)prop-1-yn-1-yl)-3-(2,2,2-trifluoroethyl)benzo[b]thiophen-7-yl)-1-methylpiperidin-4-amine (11) as yellow oil. LCMS: m/z=584 [M+1]⁺.

¹H NMR (400 MHZ, DMSO) δ 7.54 (d, J=8.6 Hz, 1H), 7.49 (s, 1H), 7.25 (t, J=7.7 Hz, 1H), 7.14 (d, J=8.2 Hz, 1H), 7.00 (d, J=8.4 Hz, 1H), 6.76 (d, J=6.4 Hz, 1H), 6.66 (d, J=7.7 Hz, 1H), 5.97 (s, 1H), 5.84 (s, 1H), 5.30) (d, J=7.9 Hz, 1H), 4.40) (d, J=6.1 Hz, 2H), 3.86-3.76 (m, 2H), 3.11 (s, 3H), 2.75 (d, J=11.1 Hz, 2H), 2.16 (s, 3H), 1.98 (t, J=11.7 Hz, 2H), 1.87 (d, J=10.5 Hz, 2H), 1.53 (d, J=12.6 Hz, 2H), 1.23 (s, 1H).

### Example 12

### N-(2-(3-((2-(2,2-difluoroethoxy)-4-(methylsulfonyl)phenyl)amino)prop-yn-1-yl)-3-(2,2,2-trifluoroethyl)benzo[b]thiophen-yl-1-methyplpiperidin-4-amine (12)

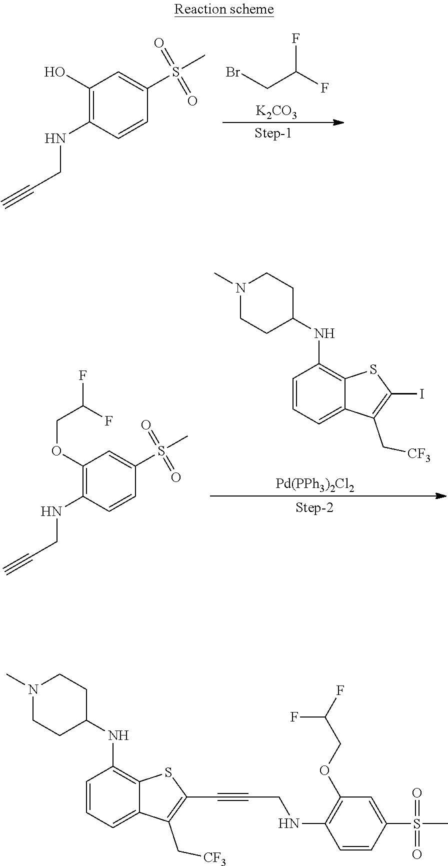

### Experimental Details

### Step 1. 2-(2,2-difluoroethoxy)-4-(methylsulfonyl)-N-(prop-2-yn-1-yl)aniline

Into a 4 mL flask purged and maintained with nitrogen atmosphere was placed 5-(methylsulfonyl)-2-(prop-2-yn-1-ylamino)phenol (0.101 g, 448.36 μmol), 2-bromo-1,1-difluoroethane (0.094 g, 648.52 μmol), K₂CO₃ (0.069 g, 499.26 μmol), DMF (1 mL). The reaction was warmed to 50° C. and stirred for 2.5 h. LCMS showed the reaction was complete. The reaction purified with Cis column, eluted with ACN/water (v/v=1/3) to afford 2-(2,2-difluoroethoxy)-4-(methylsulfonyl)-N-(prop-2-yn-1-yl)aniline (0.072 g, 248.88 μmol, 55.51% yield) as a light yellow oil. LCMS: m/z=290[M+1]⁺.

### Step 2. N-(2-(3-((2-(2,2-difluoroethoxy)-4-(methylsulfonyl)phenyl)amino)prop-1-yn-1-yl)-3-(2,2,2-trifluoroethyl)benzo[b]thiophen-7-yl)-1-methylpiperidin-4-amine (12)

Into a 4 mL flask purged and maintained with nitrogen atmosphere was placed N-(2-iodo-3-(2,2,2-trifluoroethyl)benzo[b]thiophen-7-yl)-1-methylpiperidin-4-amine (0.048 g, 105.66 μmol), 2-(2,2-difluoroethoxy)-4-(methylsulfonyl)-N-(prop-2-yn-1-yl)aniline (0.054 g, 186.66 μmol), Pd(PPh₃)₂Cl₂ (0.013 g, 18.42 μmol), CuI (0.003 g, 15.75 μmol), TEA (0.025 g, 247.06 μmol), DMF (0.5 mL), and stirred for 1 h at room temperature. L CMS showed the reaction was complete. The reaction was quenched with water (4 mL), extracted with E A (2 mL×2). The combined organic layers were washed with water (2 mL) and brine (2 mL) successively, separated, then concentrated with vacuum. The residue was purified with prep-HPLC (Mobile Phase A: water (ammonium hydroxide), Mobile Phase B: ACN; Flow rate: 40 mL/min; Gradient: 44-80-100% B (2-22-40 min); 220 nm; RT: 20.738-23.498 min) to afford N-(2-(3-((2-(2,2-difluoroethoxy)-4-(methylsulfonyl)phenyl)amino)prop-1-yn-1-yl)-3-(2,2,2-trifluoroethyl)benzo[b]thiophen-7-yl)-1-methylpiperidin-4-amine (12) (0.021 g, 34.11 μmol, 18.27% yield) as off-white solid. LCMS: m/z=616[M+1]⁺.

¹H NMR (400 MHz, DMSO-d₆) δ 7.44 (d, J=8.4 Hz, 1H), 7.37 (s, 1H), 7.25 (t, J=7.9 Hz, 1H), 7.14 (d, J=8.0 Hz, 1H), 6.94 (d, J=8.4 Hz, 1H), 6.66 (d, J=7.8 Hz, 1H), 6.58-6.27 (m, 2H), 5.32 (d, J=7.9 Hz, 1H), 4.49-4.39 (m, 4H), 3.81 (q, J=11.1 Hz, 2H), 3.10 (s, 3H), 2.78 (d, J=11.2 Hz, 2H), 2.18 (s, 3H), 2.02 (t, J=11.5 Hz, 2H), 1.93-1.84 (m, 2H), 1.60-1.48 (m, 2H).

### Example 13

### 1-methyl-N-(2-(3-((4-(methylsulfonyl)-2-(2,2,2-trifluoroethoxy)phenyl)amino)prop-1-yn-1-yl)-3-(2,2,2-trifluoroethyl)benzo[b]thiophen-7-yl)piperidin-4-amine (13)

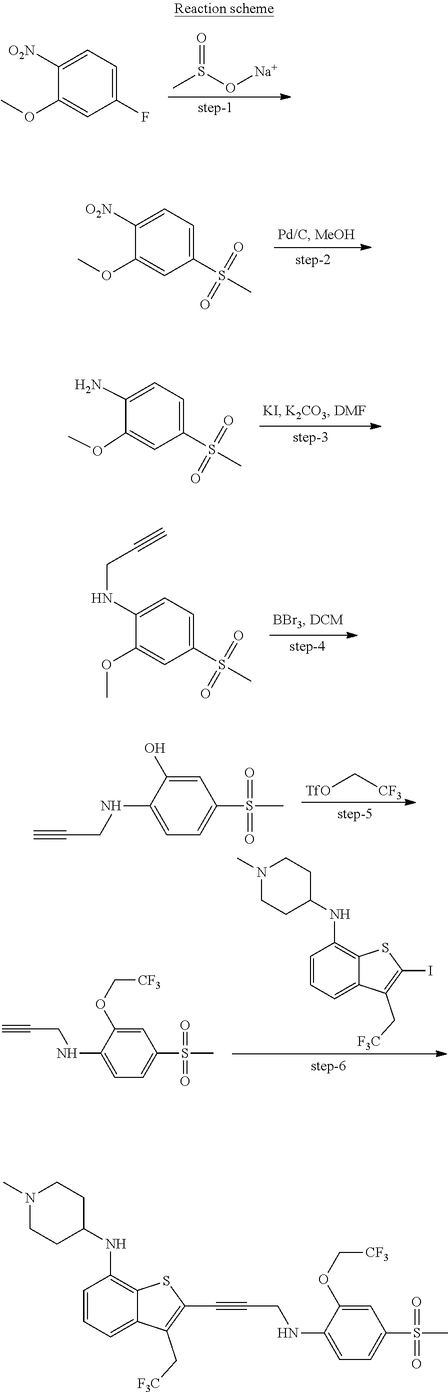

### Experimental Details

### Step 1. 2-methoxy-4-(methylsulfonyl)-1-nitrobenzene

Into a 500 mL flask purged and maintained with an inert atmosphere of nitrogen, was placed 4-fluoro-2-m ethoxy-1-nitrobenzene (20.27 g, 118.45 mmol), sodium methanesulfinate (12.62 g, 123.62 mmol), DMA (200 mL). The reaction mixture was stirred at 70° C. for 2 h. The reaction mixture was quenched with H₂O (400 mL), extracted with EA (1000 mL×2), washed with NaCl (aq. 500 mL×3) and concentrated under vacuum. Then MTBE (200 mL) was added, the mixture was stirred at room temperature for 1 h and filter ed. This resulted in 24.96 g (91.13% yield) of 2-methoxy-4-(methylsulfonyl)-1-nitrobenzene as yellow solid. LCMS: m/z=232 [M+1]⁺.

### Step 2. 2-methoxy-4-(methylsulfonyl)aniline

Into a 2 L flask purged and maintained with an inert atmosphere of hydrogen, was placed 2-(methylsulfonyl)-5-nitropyridine (24.24 g, 104.83 mmol), Pd/C (22.32 g, 209.74 mmol), methanol (1 L). The reaction mixture was stirred at room temperature for 19 h. Then the catalyst was removed by filtration and the filtrate was concentrated under vacuum. This resulted in 15.79 g (74.85% yield) of 2-methoxy-4-(methylsulfonyl)aniline as yellow solid. LCMS: m/z=173 [M+1]⁺.

### Step 3. 2-methoxy-4-(methylsulfonyl)-N-(prop-2-yn-1-yl)aniline

Into a 500 mL flask purged and maintained with an inert atmosphere of nitrogen, was placed 3-bromoprop yne (6.57 g, 55.23 mmol), 2-methoxy-4-(methylsulfonyl)aniline (10.02 g, 49.79 mmol), sodium iodide (15.58 g, 103.94 mmol), potassium carbonate (21.39 g, 154.7695 mmol), N,N-dimethylformamide (200 mL). The reaction mixture was stirred at 85° C. for 3 h. The reaction was quenched by the addition of water (500 mL). The resulting solution was extracted with EA (2×500 mL). The organic layer was combined, washed with brine (500 mL), dried over anhydrous Na₂SO₄ and concentrated under vacuum. This resulted in 7.56 g (63.45% yield) of 2-methoxy-4-(methylsulfonyl)-N-(prop-2-yn-1-yl)aniline as yellow oil. LCMS: m/z=211 [M+1]⁺.

### Step 4. 5-(methylsulfonyl)-2-(prop-2-yn-1-ylamino)phenol

Into a 100 mL flask purged and maintained with an inert atmosphere of nitrogen, was placed 2-methoxy-4-(methylsulfonyl)-N-(prop-2-yn-1-yl)aniline (2.35 g, 9.82 mmol), dichloromethane (20 mL), then tribromoboron (20 mL) was added at −10° C. The reaction mixture was stirred at 0° C. for 1 h. Then 1 M sodium hydr oxide aqueous solution was added to the mixture until pH=11˜12. The aqueous layer was collected, Then 2 M hydrochloric acid aqueous solution was added to the mixture until pH=7-8, and extracted with EA (2×100 mL). The organic layer was combined, washed with brine (100 mL), dried over anhydrous Na₂SO₄ and concentrated under vacuum. The residue was applied onto a silica gel column eluted with EA/hexane (v/v=3/5).

This resulted in 1.275 g (57.63% yield) of 5-(methylsulfonyl)-2-(prop-2-yn-1-ylamino)phenol as yellow solid. LCMS: m/z=226 [M+1]⁺.

### Step 5. 4-(methylsulfonyl)-N-(prop-2-yn-1-yl)-2-(2,2,2-trifluoroethoxy)aniline

Into a 8 mL flask was placed 5-(methylsulfonyl)-2-(prop-2-yn-1-ylamino)phenol (0.102 g, 452.80 μmol), 1,1,1-trifluoro-2-bromoethane (0.084 g, 515.54 μmol), potassium carbonate (0.126 g, 911.69 μmol), N,N-dimeth ylformamide (2 mL). The reaction mixture was stirred at 45° C. for 3 h. The reaction was quenched by the addition of water (2 mL) and extracted with EA (2×5 mL). The organic layers was combined, washed with brine (10 mL), dried over anhydrous Na₂SO₄ and concentrated under vacuum. The residue was applied onto a silica gel column eluted with EA/heptane (v/v=1/1). This resulted in 0.098 g (70.43% yield) of 4-(methylsulfonyl)-N-(prop-2-yn-1-yl)-2-(2,2,2-trifluoroethoxy)aniline as yellow oil. LCMS: m/z=308 [M+1]⁺

### Step 6. 1-methyl-N-(2-(3-((4-(methylsulfonyl)-2-(2,2,2-trifluoroethoxy)phenyl)amino)prop-1-yn-1-yl)-3-(2,2,2-trifluoroethyl)benzo[b]thiophen-7-yl)piperidin-4-amine (13)

Into a 8 mL flask purged and maintained with an inert atmosphere of nitrogen, was placed N-(2-iodo-3-(2,2,2-trifluoroethyl)benzo[b]thiophen-7-yl)-1-methylpiperidin-4-amine (0.045 g, 99.06 μmol), cuprous iodide (0.009 g, 47.26 μmol), 4-(methylsulfonyl)-N-(prop-2-yn-1-yl)-2-(2,2,2-trifluoroethoxy)aniline (0.036 g, 117.15 μmol), bis(triphenylphosphine)palladium(II) chloride (0.026 g, 36.83 μmol), triethylamine (0.022 g, 217.41 μmol), methyl sulfoxide (2 mL). The reaction mixture was stirred at room temperature for 2 h. The reaction was quenched by the addition of water (2 mL) and extracted with EA (2×4 mL). The organic layers was combined, washed with brine (5 mL), dried over anhydrous Na₂SO₄ and concentrated under vacuum. The mixture was purified by preparative HPLC (Mobile Phase A: water (10 mmol/L ammonium hydroxide), Mobile Phase B: MeOH; Flow rate: 40 mL/min; Gradient: 45-80-100% B (2-30-60 min); 270 nm; RT: 33.274-34.355 min). This resulted in 0.023 g (35.10% yield) of 1-methyl-N-(2-(3-((4-(methylsulfonyl)-2-(2,2,2-trifluoroethoxy)phenyl)amino)prop-1-yn-1-yl)-3-(2,2,2-trifluoroethyl)benzo[b]thiophen-7-yl)piperidin-4-amine (13) as yellow oil. LCMS: m/z=634 [M+1]⁺.

¹H NMR (400 MHz, DMSO) δ 7.69 (d, J=13.5 Hz, 1H), 7.50-7.42 (m, 2H), 7.25 (t, J=7.8 Hz, 1H), 7.14 (d, J=7.8 Hz, 1H), 6.96 (d, J=8.2 Hz, 1H), 6.66 (d, J=8.0 Hz, 1H), 6.44 (t, J=6.1 Hz, 1H), 5.31 (d, J=7.8 Hz, 1H), 4.89 (q, J=8.8 Hz, 2H), 4.42 (d, J=6.0 Hz, 2H), 4.22 (t, J=6.5 Hz, 1H), 3.81 (q, J=11.3 Hz, 2H), 3.10 (s, 3H), 2.76 (d, J=11.2 Hz, 2H), 2.17 (s, 3H), 1.88 (d, J=11.9 Hz, 2H), 1.54 (d, J=11.4 Hz, 2H), 1.37 (dd, J=14.9, 7.5 Hz, 1H).

### Example 14

### N-[2-[3-[2-(2-methoxyethoxy)-4-methylsulfonyl-anilino]prop-1-ynyl]-3-(2,2,2-trifluoroethyl)benzothiophen-7-yl]-1-methyl-piperidin-4-amine (14)

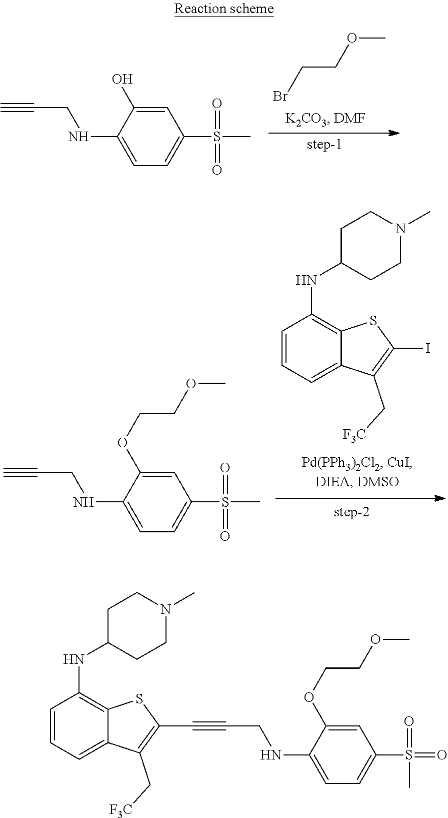

### Experimental Details

### Step 1. 2-(2-methoxyethoxy)-4-methylsulfonyl-N-prop-2-ynyl-aniline

Into a 20 mL vial was placed 5-(methylsulfonyl)-2-(prop-2-yn-1-ylamino)phenol (0.100 g, 443.92 μmol), 1-bromo-2-methoxyethane (0.074 g, 532.41 μmol), potassium carbonate (0.204 g, 1.48 mmol), dimethylformamide (4 mL). The reaction was stirred at 50° C. overnight. The reaction was quenched with water (50 mL), extracted with EA (3×30 mL). The organic layer was combined, washed with brine (50 mL), dried over anhydrous sodium sulfate, and concentrated under vacuum. The crude was purified by silica gel column eluted with EA/hexane (v/v=1/1). This resulted in 0.113 g (89.84% yield) of 2-(2-methoxyethoxy)-4-methylsulfonyl-N-prop-2-ynyl-aniline as off-white solid. LCMS: m/z=284 [M+1]⁺.

### Step 2. N-[2-[3-[2-(2-methoxyethoxy)-4-methylsulfonyl-anilino]prop-1-ynyl]-3-(2,2,2-trifluoroethyl)benzothiophen-7-yl]-1-methyl-piperidin-4-amine (14)

Into a 8 mL vial was placed 2-(2-methoxyethoxy)-4-methylsulfonyl-N-prop-2-ynyl-aniline (0.049 g, 172.94 μmol), N-(2-iodo-3-(2,2,2-trifluoroethyl)benzo[b]thiophen-7-yl)-1-methylpiperidin-4-amine (0.050 g, 110.06 μmol), bis(triphenylphosphine)palladium(II) chloride (0.076 g, 107.66 μmol), CuI (0.140 g, 735.10 μmol), DIEA (0.209 g, 1.62 mmol), methyl sulfoxide (3 mL). The reaction was stirred at RT for 3 h under nitrogen. The reaction was quenched with water (20 mL), extracted with EA (3×30 mL). The organic layers was combined, washed with brine (50 mL), dried over anhydrous sodium sulfate, filtered and concentrated under vacuum. The mixture was purified by preparative HPLC (Mobile Phase A: water (10 mmol/L TFA), Mobile Phase B: ACN; Flow rate: 40 mL/min; Gradient: 15-40-50% B2-30-40 min); 270 nm; RT: 36.17-36.91 min) to provide the desired product. This resulted in 0.014 g (20.86% yield) of N-[2-[3-[2-(2-methoxyethoxy)-4-methylsulfonyl-anilino]prop-1-ynyl]-3-(2,2,2-trifluoroethyl)benzothiophen-7-yl]-1-methyl-piperidin-4-amine (14) as off-white solid. LCMS: m/z=610 [M+1]⁺.

¹H NMR (400 MHz, MeOD) δ 7.52 (d, J=8.4 Hz, 1H), 7.36 (s, 1H), 7.29 (t, J=7.9 Hz, 1H), 7.15 (d, J=8.0 Hz, 1H), 6.97 (d, J=8.4 Hz, 1H), 6.73 (d, J=7.2 Hz, 1H), 4.43 (s, 2H), 4.33-4.21 (m, 2H), 3.87-3.77 (m, 2H), 3.77-3.64 (m, 2H), 3.58-3.49 (m, 1H), 3.47 (s, 3H), 3.06 (d, J=10.9 Hz, 3H), 2.95 (d, J=11.5 Hz, 2H), 2.36 (s, 3H), 2.29 (t, J=11.2 Hz, 2H), 2.09 (d, J=12.5 Hz, 2H), 1.73-1.58 (m, 2H).

### Example 15

### (4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)dimethylphosphine oxide (racemic) (15)

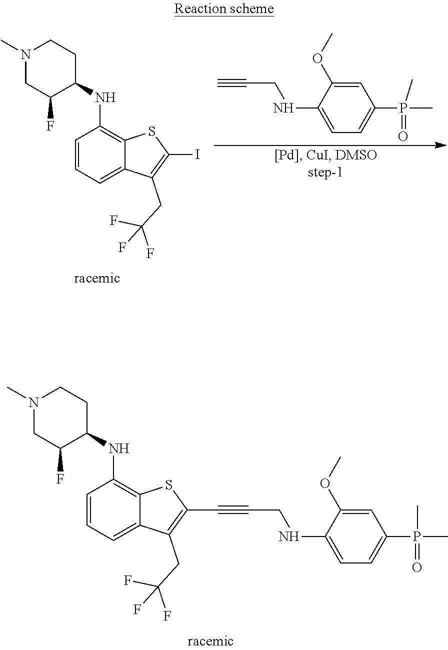

### Experimental Details

### Step 1 (4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)dimethylphosphine oxide (racemic) (15)

Into a 10-mL round-bottom flask was placed (3-methoxy-4-(prop-2-yn-1-ylamino)phenyl)dimethylphosphine oxide (0.078 g, 328.78 μmol), (Z)-3-fluoro-1-methyl-N-(3-(2,2,2-trifluoroethyl)benzo[b]thiophen-7-yl)piperidin-4-amine (racemic) (0.076 g, 160.92 μmol), Pd(PPh₃)₂Cl₂ (0.023 g, 32.58 μmol), CuI (0.020 g, 105.01 μmol), DIEA (0.086 g, 665.41 umol), methyl sulfoxide (2 mL). The reaction was stirred under nitrogen atmosphere at RT for 6 h. The reaction was quenched with water (5 mL). The resulted solution was extracted with EA (3×10 mL), washed with brine (10 mL), dried over anhydrous sodium sulfate, filtered and concentrated under vacuum. The crude product was purified by preparative HPLC (Mobile Phase A: water (0.1% ammonium hydroxide), Mobile Phase B: ACN; Flow rate: 70 mL/min; Gradient: 35-70-70% B (2-30-60 min); 270 nm; RT: 25.18-26.87) to provide the desired product. This resulted in 0.047 g (80.81 μmol, 24.57% yield) of (4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)dimethylphosphine oxide (racemic) (15) as white solid. LCMS: m/z=582 [M+1]⁺.

¹H NMR (400 MHz, DMSO-d₆) δ 7.32-7.17 (m, 3H), 7.15-7.09 (m, 1H), 6.85-6.79 (m, 1H), 6.78 (d, J=7.7 Hz, 1H), 6.06 (t, J=6.4 Hz, 1H), 5.15 (d, J=8.5 Hz, 1H), 4.79 (d, J=49.5 Hz, 1H), 4.34 (d, J=6.3 Hz, 2H), 3.81 (d, J=29.5 Hz, 5H), 3.64 (d, J=28.8 Hz, 1H), 3.03 (t, J=11.4 Hz, 1H), 2.79 (d, J=11.6 Hz, 1H), 2.27 (d, J=13.0 Hz, 1H), 2.18 (s, 3H), 2.08 (t, J=11.4 Hz, 1H), 1.99-1.90 (m, 1H), 1.71 (d, J=11.8 Hz, 1H), 1.58 (d, J=13.1 Hz, 6H).

### Example 16

### N1-(2-(3-((2-methoxy-4-(methylsulfonyl)phenyl)amino)prop-1-yn-1-yl)-3-(2,2,2-trifluoroethyl)benzo[b]thiophen-7-yl)-N4,N4-dimethylcyclohexane-1,4-diamine (16)

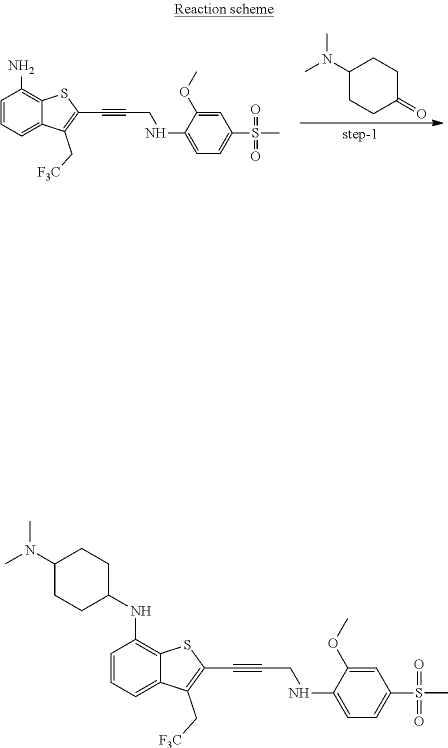

### Experimental Details

### Step 1. N1-(2-(3-((2-methoxy-4-(methylsulfonyl)phenyl)amino)prop-1-yn-1-yl)-3-(2,2,2-trifluoroethyl)benzo[b]thiophen-7-yl)-N4,N4-dimethylcyclohexane-1,4-diamine (16)

Into a 50-mL round-bottom flask was placed 2-(3-((2-methoxy-4-(methylsulfonyl)phenyl)amino)prop-1-yn-1-yl)-3-(2,2,2-trifluoroethyl)benzo[b]thiophen-7-amine (0.083 g, 177.15 μmol), 4-(dimethylamino)cyclohexan-1-one (0.120 g, 849.80 μmol), NaCNBH₃ (0.079 g, 1.84 mmol), and EtOH (5 mL). The mixture was stirred at 60° C. for 16 h. The reaction was quenched by water (20 mL), extracted with EA (50 mL×2). The combined organic layer was washed with water (50 mL) and brine (50 mL) successively, separated and concentrated under vacuum. The mixture was purified by preparative HPLC (Mobile Phase A: water (10 mmoL/L ammonium hydroxide), Mobile Phase B: ACN; Flow rate: 70 mL/min; Gradient: 60-90-100% B (2-30-60 min); 265 nm; RT: 33.497-37.464 min). This resulted in 47 mg (44% yield) of N1-(2-(3-((2-methoxy-4-(methyl sulfonyl)phenyl)amino)prop-1-yn-1-yl)-3-(2,2,2-trifluoroethyl)benzo[b]thiophen-7-yl)-N4,N4-dimethylcyclohexane-1,4-diamine (16) as white solid. LCMS: m/z=594 [M+1]⁺.

¹H NMR (400 MHz, DMSO) δ 7.39 (d, J=8.3 Hz, 1H), 7.24 (d, J=10.9 Hz, 2H), 7.13 (t, J=7.5 Hz, 1H), 6.89 (d, J=8.4 Hz, 1H), 6.67 (t, J=8.5 Hz, 1H), 6.51 (t, J=6.1 Hz, 1H), 5.21 (dd, J=40.1, 7.5 Hz, 1H), 4.38 (d, J=6.1 Hz, 2H), 3.90 (s, 3H), 3.56 (s, 2H), 3.10 (s, 3H), 2.21 (s, 3H), 2.18 (s, 3H), 2.05 (d, J=18.8 Hz, 2H), 1.76 (d, J=7.6 Hz, 3H), 1.61 (s, 1H), 1.48 (s, 1H), 1.33 (d, J=14.5 Hz, 2H).

### Example 17

### Trans-N-(4-(2-oxa-6-azaspiro[3.3]heptan-6-yl)cyclohexyl)-2-(3-((2-methoxy-4-(methylsulfonyl)phenyl)amino) prop-1-yn-1-yl)-3-(2,2,2-trifluoroethyl)benzo[b]thiophen-7-amine (17) and cis-N-(4-(2-oxa-6-azaspiro[3.3]heptan-6-yl)cyclohexyl)-2-(3-((2-methoxy-4-(methylsulfonyl)phenyl)amino)prop-1-yn-1-yl)-3-(2,2,2-trifluoroethyl)benzo[b]thiophen-7-amine (17b)

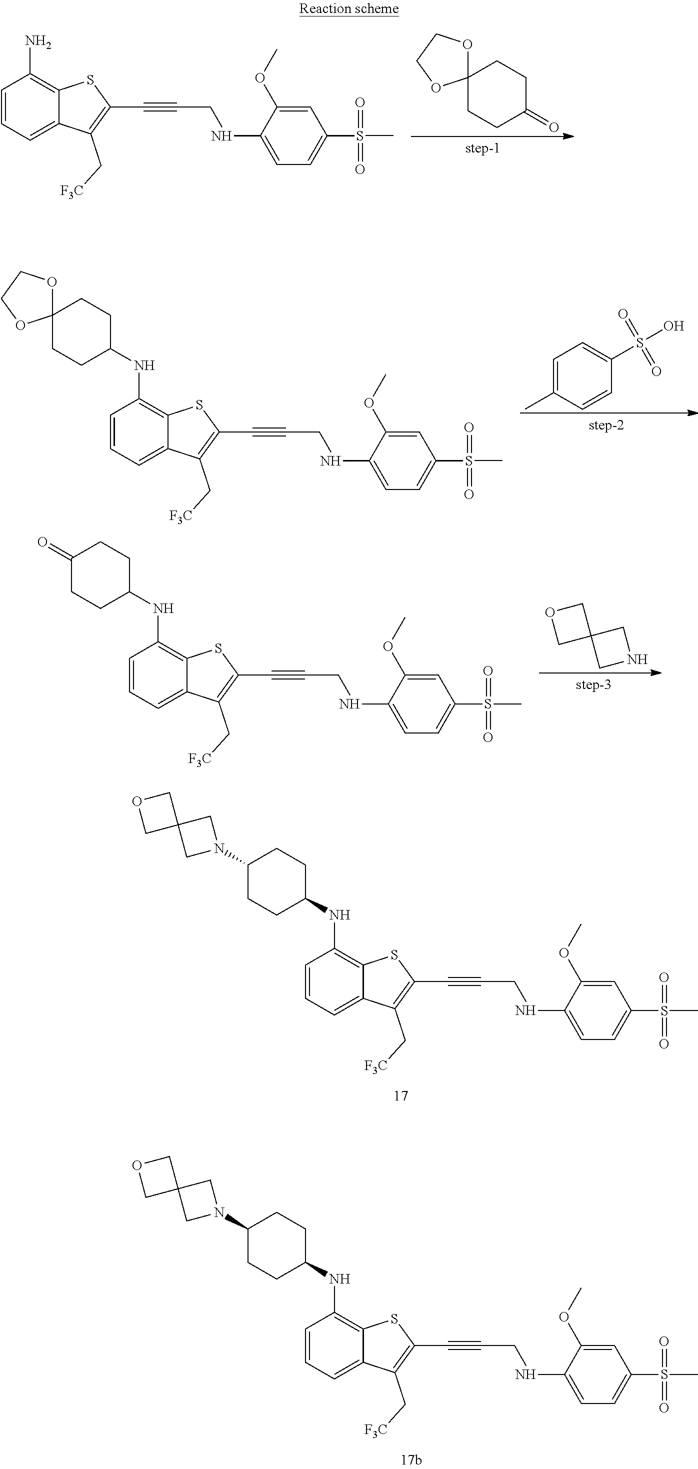

### Experimental Details

### Step 1. N-(2-(3-((2-methoxy-4-(methylsulfonyl)phenyl)amino)prop-1-yn-1-yl)-3-(2,2,2-trifluoroethyl)benzo[b]thiophen-7-yl)-1,4-dioxaspiro[4.5]decan-8-amine

Into a 25 nL flask was placed titanium ethoxide (0.577 g, 2.53 mmol), 2-(3-((2-methoxy-4-(methylsulfonyl) phenyl)amino)prop-1-yn-1-yl)-3-(2,2,2-trifluoroethyl)benzo[b]thiophen-7-amine (0.401 g, 855.90 μmol), toluene (10 mL), 1,4-dioxaspiro[4.5]decan-8-one (0.437 g, 2.80 mmol). The reaction mixture was stirred at 110° C. for 2 h. The reaction was concentrated under vacuum and dissolved with methanol (10 mL). Then sodium cyanoboronhydride (0.226 g, 5.27 mmol) was added. The reaction mixture was stirred at room temperature for another 15 h. The reaction was quenched by the addition of water (20 mL) and extracted with EA (2×20 mL). The organic layer was combined, washed with brine (20 mL), dried over anhydrous Na₂SO₄ and concentrated under vacuum. The residue was applied onto a silica gel column eluted with EA/heptane (v/v=2/5). This resulted in 0.608 g (93.60% yield) of N-(2-(3-((2-methoxy-4-(methylsulfonyl)phenyl)amino)prop-1-yn-1-yl)-3-(2,2,2-trifluoroethyl)benzo[b]thiophen-7-yl)-1,4-dioxaspiro[4.5]decan-8-amine as yellow oil. LCMS: m/z=609 [M+1]⁺.

### Step 2. 4-((2-(3-((2-methoxy-4-(methylsulfonyl)phenyl)amino)prop-1-yn-1-yl)-3-(2,2,2-trifluoroethyl)benzo[b]thiophen-7-yl)amino)cyclohexan-1-one

Into a 25 mL flask purged and maintained with an inert atmosphere of nitrogen, was placed N-(2-(3-((2-m ethoxy-4-(methylsulfonyl)phenyl)amino)prop-1-yn-1-yl)-3-(2,2,2-trifluoroethyl)benzo[b]thiophen-7-yl)-1,4-dioxaspiro[4.5]decan-8-amine (0.603 g, 990.65 μmol), acetonitrile (10 mL), 4-methylbenzenesulfonic acid hydrate (1.583 g, 8.32 mmol) in water (5 mL). The reaction mixture was stirred at room temperature for 5 h. Then saturated sodium hydrogen carbonate aqueous solution was added to the mixture until pH=7-8. The resulting solution extracted with EA (2×20 mL). The organic layers was combined, washed with brine (50 mL), dried over anhydrous Na₂SO₄ and concentrated under vacuum. The residue was applied onto a silica gel column eluted with EA/heptane (v/v=3/1). This resulted in 0.447 g (79.91% yield) of 4-((2-(3-((2-methoxy-4-(methylsulfonyl)phenyl)amino)prop-1-yn-1-yl)-3-(2,2,2-trifluoroethyl)benzo[b]thiophen-7-yl)amino)cyclohexan-1-one as yellow oil. LCMS: m/z=565 [M+1]⁺.

### Step 3. trans-N-(4-(2-oxa-6-azaspiro[3.3]heptan-6-yl)cyclohexyl)-2-(3-((2-methoxy-4-(methylsulfonyl)phenyl)amino)prop-1-yn-1-yl)-3-(2,2,2-trifluoroethyl)benzo[b]thiophen-7-amine (17) and cis-N-(4-(2-oxa-6-azaspiro[3.3]heptan-6-yl)cyclohexyl)-2-(3-((2-methoxy-4-(methylsulfonyl)phenyl)amino)prop-1-yn-1-yl)-3-(2,2,2-trifluoroethyl)benzo[b]thiophen-7-amine (17b)

Into a 8 mL flask was placed 2-oxa-6-azaspiro[3.3]heptane (0.100 g, 1.01 mmol), 4-((2-(3-((2-methoxy-4-(methylsulfonyl)phenyl)amino)prop-1-yn-1-yl)-3-(2,2,2-trifluoroethyl)benzo[b]thiophen-7-yl)amino)cyclohexan-1-one (0.050 g, 88.55 μmol), methanol (2 mL), acetic acid (0.1 mL). The reaction mixture was stirred at room temperature for 24 h. Then sodium cyanoboronhydride (0.030 g, 699.70 μmol) was added. The reaction mixture was stirred at room temperature for another 2 h. The reaction was quenched by the addition of water (10 mL) and extracted with EA (2×20 mL). The organic layers was combined, washed with brine (20 mL) and concentrated under vacuum. The mixture was purified by preparative HPLC (Mobile Phase A: water (10 mmoL/L ammonium hydroxide), Mobile Phase B: MeOH; Flow rate: 70 mL/min; Gradient: 40-75-100% B (2-30-60 min; 272 nm; RT: 33.268-34.375 min). This resulted in 0.017 g (29.63% yield) of trans-N-(4-(2-oxa-6-azaspiro[3.3]heptan-6-yl)cyclohexyl)-2-(3-((2-methoxy-4-(methylsulfonyl)phenyl)amino)prop-1-yn-1-yl)-3-(2,2,2-trifluoroethyl)benzo[b]thiophen-7-amine (17) as white solid. LCMS: m/z=648 [M+1]⁺.

¹H NMR (400 MHz, DMSO) δ 7.39 (d, J=8.9 Hz, 1H), 7.27-7.21 (m, 2H), 7.11 (d, J=8.0 Hz, 1H), 6.88 (d, J=8.3 Hz, 1H), 6.63 (d, J=7.9 Hz, 1H), 6.50 (s, 1H), 5.23 (d, J=8.2 Hz, 1H), 4.58 (s, 4H), 4.37 (d, J=6.5 Hz, 2H), 3.89 (s, 3H), 3.79 (d, J=11.5 Hz, 2H), 3.21 (s, 4H), 3.09 (s, 3H), 1.92 (s, 2H), 1.69 (s, 2H), 1.25 (d, J=14.6 Hz, 3H), 0.99 (d, J=13.7 Hz, 3H).

And 0.010 g (17.43% yield) of cis-N-(4-(2-oxa-6-azaspiro[3.3]heptan-6-yl)cyclohexyl)-2-(3-((2-methoxy-4-(methylsulfonyl)phenyl)amino)prop-1-yn-1-yl)-3-(2,2,2-trifluoroethyl)benzo[b]thiophen-7-amine (17b) as white solid. LCMS: m/z=648 [M+1]⁺.

¹H NMR (400 MHz, DMSO) δ 7.39 (d, J=8.7 Hz, 1H), 7.29-7.19 (m, 2H), 7.11 (d, J=7.9 Hz, 1H), 6.88 (d, J=8.5 Hz, 1H), 6.63 (d, J=7.8 Hz, 1H), 6.51 (t, J=6.1 Hz, 1H), 5.27 (d, J=7.8 Hz, 1H), 4.59 (s, 4H), 4.38 (d, J=6.1 Hz, 2H), 3.89 (s, 3H), 3.79 (q, J=10.8 Hz, 2H), 3.20 (s, 4H), 3.09 (s, 3H), 2.09 (d, J=15.6 Hz, 1H), 1.57 (dd, J=31.9, 18.2 Hz, 6H), 1.39 (d, J=10.3 Hz, 2H), 1.23 (s, 1H).

### Example 18

### Trans-N-(4-(7-oxa-2-azaspiro[3.5]nonan-2-yl)cyclohexyl)-2-(3-((2-methoxy-4-(methylsulfonyl)phenyl)amino)prop-1-yn-1-yl)-3-(2,2,2-trifluoroethyl)benzo[b]thiophen-7-amine (18) and cis-N-(4-(7-oxa-2-azaspiro[3.5]nonan-2-yl)cyclohexyl)-2-(3-((2-methoxy-4-(methylsulfonyl)phenyl)amino)prop-1-yn-1-yl)-3-(2,2,2-trifluoroethyl) benzo[b]thiophen-7-amine (18b)

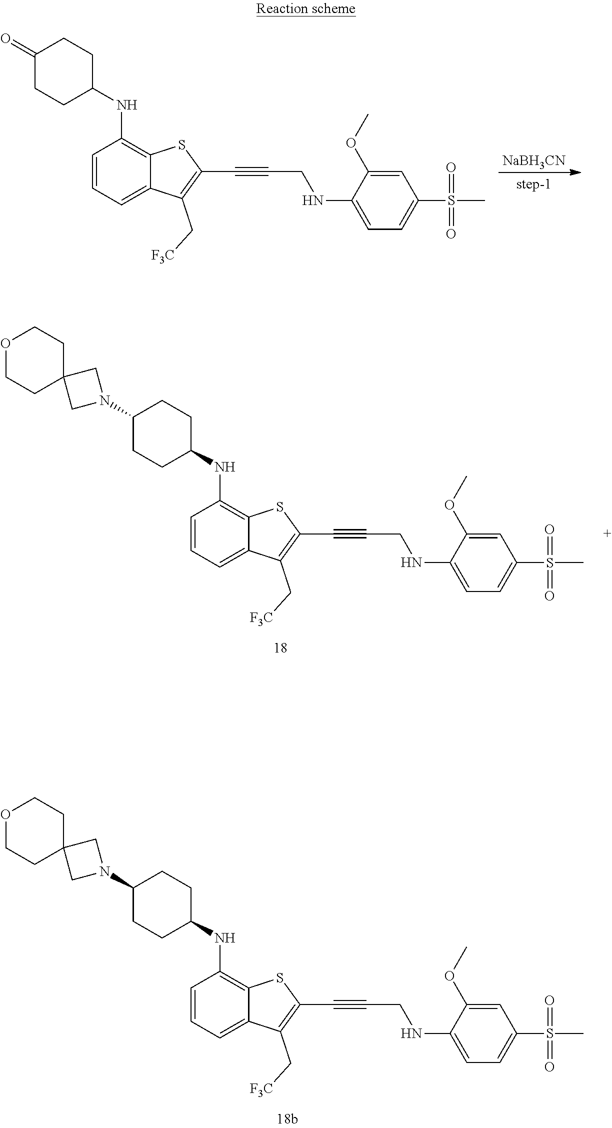

### Step 1. trans-N-(4-(7-oxa-2-azaspiro[3.5]nonan-2-yl)cyclohexyl)-2-(3-((2-methoxy-4-(methylsulfonyl)phenyl) amino)prop-1-yn-1-yl)-3-(2,2,2-trifluoroethyl)benzo[b]thiophen-7-amine (18) and cis-N-(4-(7-oxa-2-azaspiro[3.5]nonan-2-yl)cyclohexyl)-2-(3-((2-methoxy-4-(methylsulfonyl)phenyl)amino)prop-1-yn-1-yl)-3-(2,2,2-trifluoroethyl)benzo[b]thiophen-7-amine (18b)

Into a 4-mL sealed tube and maintained with an inert atmosphere of nitrogen, was placed 4-((2-(3-((2-meth oxy-4-(methylsulfonyl)phenyl)amino)prop-1-yn-1-yl)-3-(2,2,2-trifluoroethyl)benzo[b]thiophen-7-yl)amino)cyclohexan-1-one (0.05 g, 88.55 mmol), 7-oxa-2-azaspiro[3.5]nonane (0.05 g, 393.11 mmol), acetic acid (0.03 g, 499.58 mmol), methanol (1 mL). The reaction mixture was stirred at room temperature for 3 h. Sodium cyanoborohydride (0.04 g, 636.53 mmol) was added to the reaction and stirred at room temperature for 2 h. The reaction was then quenched by the addition of water. The resulting solution was extracted with ethyl acetate (2×50 mL), the organic layers combined and dried over anhydrous Na₂SO₄, filtered and concentrated under vacuum. The resulting crude product was further purified by pre-HPLC with MeOH/H₂O (0.1% ammonium hydroxide), Flow rate: 25 mL/min; Gradient: 50-85-100% B (2-30-60 min); 270 nm; RT: 36.800-38.238/40.647-42.205). This resulted in 0.008 g (13.37% yield) of trans-N-(4-(7-oxa-2-azaspiro[3.5]nonan-2-yl)cyclohexyl)-2-(3-((2-methoxy-4-(methylsulfonyl)phenyl)amino)prop-1-yn-1-yl)-3-(2,2,2-trifluoroethyl)benzo[b]thiophen-7-amine (18) as white solid. LCMS: m/z=676 [M+1]⁺

¹H NMR (400 MHz, DMSO) δ 7.39 (d, J=8.3 Hz, 1H), 7.29-7.19 (m, 2H), 7.12 (d, J=7.8 Hz, 1H), 6.88 (d, J=8.4 Hz, 1H), 6.63 (d, J=7.7 Hz, 1H), 6.51 (t, J=6.2 Hz, 1H), 5.24 (d, J=8.0 Hz, 1H), 4.38 (d, J=6.0 Hz, 2H), 3.89 (s, 3H), 3.80 (dd, J=22.2, 11.3 Hz, 2H), 3.47 (s, 4H), 3.10 (s, 3H), 2.91 (s, 4H), 1.95 (d, J=11.1 Hz, 3H), 1.74 (d, J=11.7 Hz, 2H), 1.61 (s, 4H), 1.27 (dd, J=23.1, 11.1 Hz, 3H), 1.10-0.92 (m, 2H).

And 0.007 g (11.69% yield) of cis-N-(4-(7-oxa-2-azaspiro[3.5]nonan-2-yl)cyclohexyl)-2-(3-((2-methoxy-4-(methylsulfonyl)phenyl)amino)prop-1-yn-1-yl)-3-(2,2,2-trifluoroethyl)benzo[b]thiophen-7-amine (18b) as white solid. LCMS: m/z=676 [M+1]⁺

¹H NMR (400 MHz, DMSO) δ 7.39 (d, J=8.2 Hz, 1H), 7.31-7.19 (m, 2H), 7.12 (d, J=7.8 Hz, 1H), 6.89 (d, J=8.4 Hz, 1H), 6.64 (d, J=7.5 Hz, 1H), 6.51 (t, J=5.8 Hz, 1H), 5.22 (d, J=7.4 Hz, 1H) 4.38 (d, J=5.9 Hz, 2H), 3.89 (s, 3H), 3.79 (dd, J=22.2, 11.2 Hz, 2H), 3.49 (s, 4H), 3.10 (s, 3H), 2.89 (s, 4H), 2.22 (s, 1H), 1.71-1.51 (m, 9H), 1.40 (t, J=15.1 Hz, 2H), 1.23 (s, 2H).

### Example 19

### Trans-2,2′-((4-((2-(3-((2-methoxy-4-(methylsulfonyl)phenyl)amino)prop-1-yn-1-yl)-3-(2,2,2-trifluoroethyl)benzo[b]thiophen-7-yl)amino)cyclohexyl)azanediyl)bis(ethan-1-ol) (19) and cis-2,2′-((4-((2-(3-((2-methoxy-4-(methylsulfonyl)phenyl)amino)prop-1-yn-1-yl)-3-(2,2,2-trifluoroethyl)benzo[b]thiophen-7-yl)amino)cyclohexyl)azanediyl)bis(ethan-1-ol) (19b)

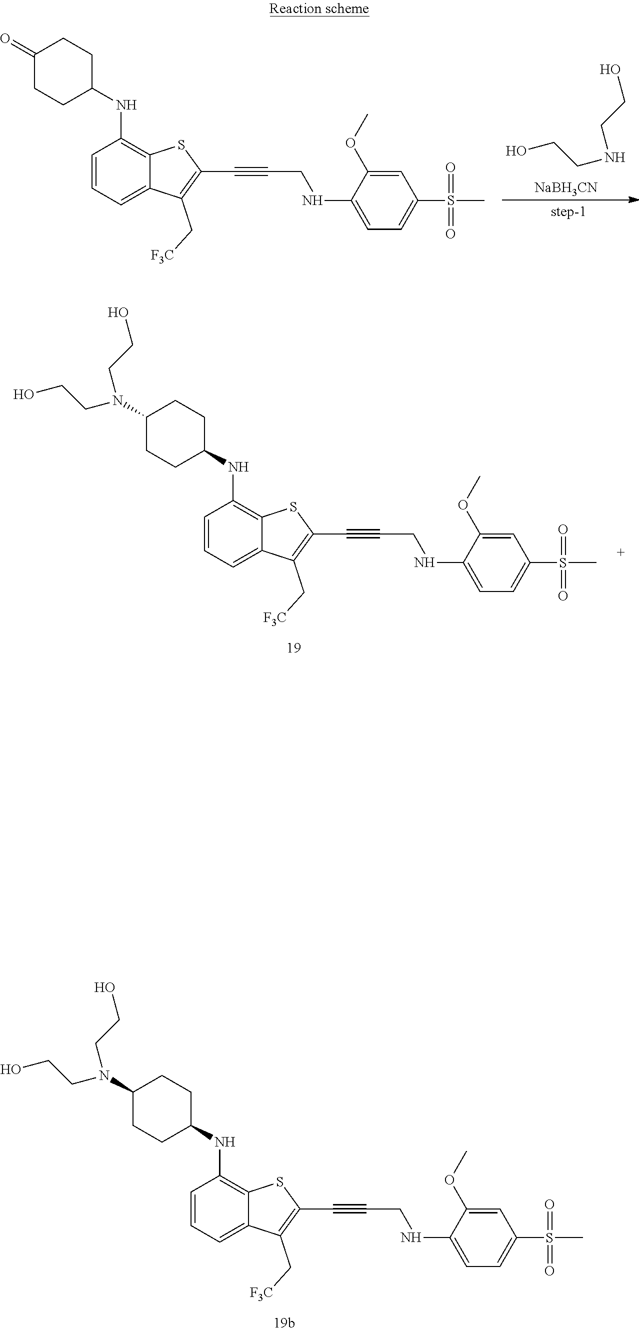

### Experimental Details

### Step 1. Synthesis of trans-2,2′-((4-((2-(3-((2-methoxy-4-(methylsulfonyl)phenyl)amino)prop-1-yn-1-yl)-3-(2,2,2-trifluoroethyl)benzo[b]thiophen-7-yl)amino)cyclohexyl)azanediyl)bis(ethan-1-ol) (19) and cis-2,2′-((4-((2-(3-((2-methoxy-4-(methylsulfonyl)phenyl)amino)prop-1-yn-1-yl)-3-(2,2,2-trifluoroethyl)benzo[b]thiophen-7-yl)amino)cyclohexyl)azanediyl)bis(ethan-1-ol) (19b)

Into a 8-mL reaction vial was added 4-((2-(3-((2-methoxy-4-(methylsulfonyl)phenyl)amino)prop-1-yn-1-yl)-3-(2,2,2-trifluoroethyl)benzo[b]thiophen-7-yl)amino)cyclohexan-1-one (0.033 g, 58.44 μmol), acetic acid, (0.033 g, 549.52 μmol), 2,2′-azanediylbis(ethan-1-ol) (0.554 g, 5.26 mmol). The reaction mixture was stirred at 60° C. for 2 h. The reaction was quenched by aq·Na₂CO₃ (10 mL), extracted with DCM (30 mL×3) and concentrated under vacuum. The mixture was purified by preparative HPLC (Mobile Phase A: water (10 mmoL/L ammonium hydroxide), Mobile Phase B: ACN; Flow rate: 40 mL/min; Gradient: 35-65-65% B (2-30-60 min); 270 nm; RT: 26.332-27.528 min and RT: 31.669-33.143 min) to afford trans-2,2′-((4-((2-(3-((2-methoxy-4-(methylsulfonyl)phenyl)amino)prop-1-yn-1-yl)-3-(2,2,2-trifluoroethyl)benzo[b]thiophen-7-yl)amino)cyclohexyl)azanediyl)bis(ethan-1-ol) (0.005 g, 13.08% yield) as off-white solid. LCMS: m/z=654 [M+1]⁺.

¹H NMR (400 MHz, DMSO) δ 7.39 (dd, J=8.3, 1.6 Hz, 1H), 7.25 (dd, J=8.3, 4.8 Hz, 2H), 7.12 (d, J=7.9 Hz, 1H), 6.89 (d, J=8.4 Hz, 1H), 6.66 (d, J=7.9 Hz, 1H), 6.51 (t, J=6.2 Hz, 1H), 5.23 (d, J=8.0 Hz, 1H), 4.44-4.23 (m, 4H), 3.89 (s, 3H), 3.80 (q, J=11.0 Hz, 2H), 3.41-3.33 (m, 4H), 3.10 (s, 3H), 2.54 (d, J=6.4 Hz, 4H), 1.74 (d, J=11.3 Hz, 2H), 1.46-1.17 (m, 6H).

And cis-2,2′-((4-((2-(3-((2-methoxy-4-(methylsulfonyl)phenyl)amino)prop-1-yn-1-yl)-3-(2,2,2-trifluoroethyl)benzo[b]thiophen-7-yl)amino)cyclohexyl)azanediyl)bis(ethan-1-ol) (0.004 g, 10.46%) as off-white solid. LCMS: m/z=654 [M+1]⁺.

¹H NMR (400 MHz, DMSO) δ 7.39 (d, J=8.3 Hz, 1H), 7.31-7.23 (m, 2H), 7.18 (d, J=8.0 Hz, 1H), 6.89 (d, J=8.4 Hz, 1H), 6.69 (d, J=7.7 Hz, 1H), 6.52 (t, J=6.1 Hz, 1H), 5.05 (d, J=5.7 Hz, 1H), 4.38 (d, J=6.1 Hz, 2H), 3.95-3.74 (m, 5H), 3.71 (s, 1H), 3.40 (t, J=6.2 Hz, 4H), 3.10 (s, 3H), 2.62 (dd, J=18.5, 12.6 Hz, 4H), 2.05-1.85 (m, 3H), 1.71-1.41 (m, 5H), 1.24 (s, 3H).

### Example 20

### N-[2-[3-(2-methoxy-4-methylsulfonyl-anilino)prop-1-ynyl]-3-(2,2,2-trifluoroethyl)benzothiophen-7-yl]-1-(3-m ethoxypropyl)piperidin-4-amine (20)

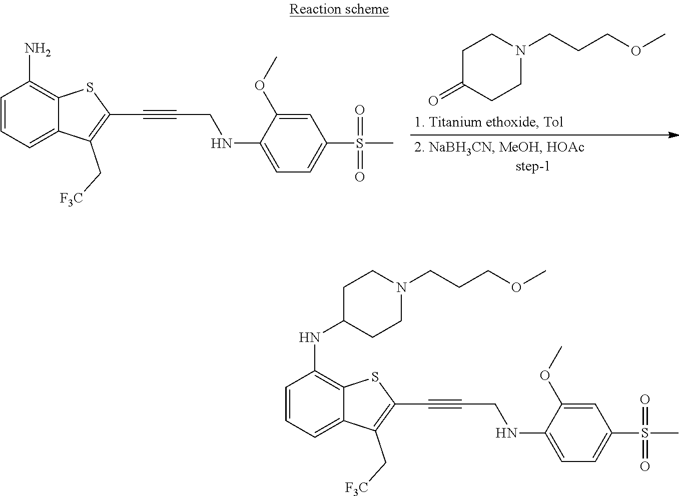 [SMILES: COc1cc(S(C)(=O)=O)ccc1NCC#Cc1sc2c(N)cccc2c1CC(F)(F)F ; COCCCN1CCC(=O)CC1 ; COCCCN1CCC(Nc2cccc3c(CC(F)(F)F)c(C#CCNc4ccc(S(C)(=O)=O)cc4OC)sc23)CC1]

### Experimental Details

### Step 1. N-[2-[3-(2-methoxy-4-methylsulfonyl-anilino)prop-1-ynyl]-3-(2,2,2-trifluoroethyl)benzothiophen-7-yl]-1-(3-methoxypropyl)piperidin-4-amine (20)

Into a 8 mL vial was placed 2-(3-((2-methoxy-4-(methylsulfonyl)phenyl)amino)prop-1-yn-1-yl)-3-(2,2,2-trifluoroethyl)benzo[b]thiophen-7-amine (0.056 g, 119.53 μmol), 1-(3-methoxypropyl)piperidin-4-one (0.125 g, 729.99 μmol), titanium ethoxide (0.156 g, 683.89 μmol), Toluene (1 mL). The reaction was stirred at 110° C. for 1 h. The reaction was concentrated under vacuum. The crude was added MeOH (1 mL) and Sodium cyanoboronhydride (0.120 g, 2.80 mmol). The reaction was stirred at r.t overnight. The reaction was quenched with water (20 mL), extracted with EA (30 mL×2). The combined organic layers were washed with w ater (50 mL) and brine (50 mL) successively, separated and concentrated under vacuum. The mixture was purified by preparative HPLC (Mobile Phase A: water (10 mmoL/L ammonium hydroxide), Mobile Phase B: MeOH; Flow rate: 70 mL/min; Gradient: 45-80-100% B (2-30-60 min); 270 nm; RT: 40.998-42.953 min). This resulted in 0.035 g (46.95% yield) of N-[2-[3-(2-methoxy-4-methylsulfonyl-anilino)prop-1-ynyl]-3-(2,2,2-tri fluoroethyl)benzothiophen-7-yl]-1-(3-methoxypropyl)piperidin-4-amine (20) as off-white solid. LCMS: m/z=624 [M+1]⁺.

¹H NMR (400 MHz, Methanol-d₄) δ 7.54-7.47 (m, 1H), 7.36-7.25 (m, 2H), 7.15 (d, J=8.0 Hz, 1H), 6.96 (d, J=8.4 Hz, 1H), 6.73 (d, J=7.6 Hz, 1H), 4.42 (s, 2H), 3.97 (s, 3H), 3.76-3.64 (m, 2H), 3.58-3.49 (m, 1H), 3.45 (d, J=6.0 Hz, 2H), 3.34 (s, 3H), 3.08 (s, 3H), 3.00 (d, J=11.6 Hz, 2H), 2.54-2.46 (m, 2H), 2.23 (d, J=23.2 Hz, 2H), 2.09 (d, J=12.2 Hz, 2H), 1.87-1.76 (m, 2H), 1.71-1.56 (m, 2H).

### Example 21

### 3-methoxy-N,N-dimethyl-4-((3-(7-((1-methylpiperidin-4-yl)amino)-3-(2,2,2-trifluoroethyl)benzo[b]thiophen-2-yl)prop-2-yn-1-yl)amino)benzenesulfonamide (21)

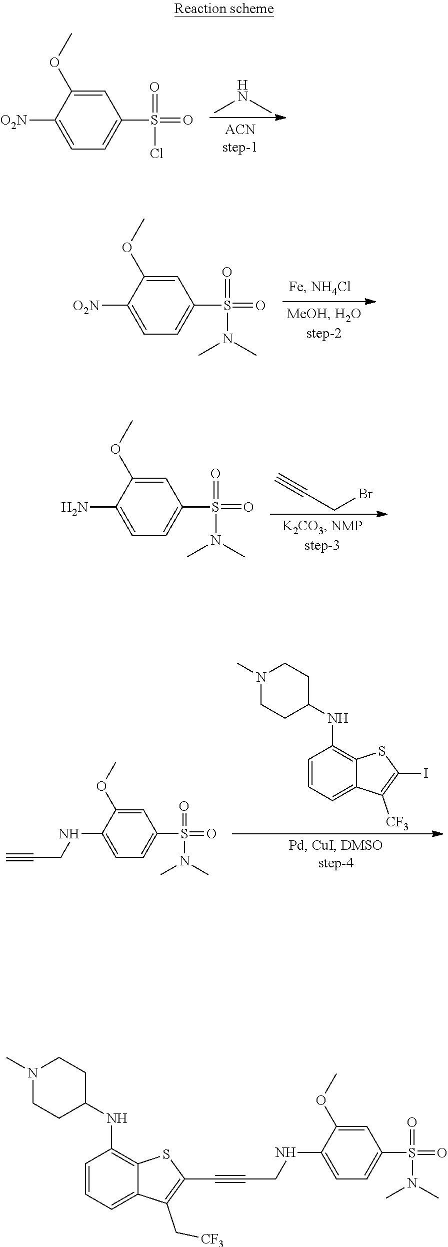

### Experimental Details

### Step 1 3-methoxy-N,N-dimethyl-4-nitrobenzenesulfonamide

Into a 20 mL sealed tube was placed 3-methoxy-4-nitrobenzenesulfonyl chloride (0.536 g, 2.13 mmol), dim ethylamine (0.542 g, 3.96 mmol), ACN (5 mL). The reaction was stirred at RT for 0.5 h. The reaction w as stirred at RT for 0.5 h. The reaction mixture was purified with silica gel column eluted with EA/hexane (v/v=2/3). This resulted in 0.554 g (2.12 mmol, 99.93% yield) of 3-methoxy-N,N-dimethyl-4-nitrobenzenesulfonamide as light yellow solid. LCMS: m/z=261 [M+1]⁺.

### Step 2 4-amino-3-methoxy-N,N-dimethylbenzenesulfonamide

Into a 40 mL sealed tube was placed 3-methoxy-N,N-dimethyl-4-nitrobenzenesulfonamide (0.566 g, 2.17 m mol), iron (1.406 g, 25.17 mmol), NH₄Cl (1.121 g, 20.95 mmol), MeOH (8 mL), water (2 mL). The reaction mixture was stirred under nitrogen at 70° C. for 4 h. The reaction mixture was filtered through celite p ad and the filter cake was washed with methanol (2×20 mL). The filterate was then concentrated under reduced pressure to afford the product. This resulted in 0.530 g (2.29 mmol, 99.93% yield) of 4-amino-3-methoxy-N,N-dimethylbenzenesulfonamide as light yellow solid. LCMS: m/z=231 [M+1]⁺.

### Step 3 3-methoxy-N,N-dimethyl-4-(prop-2-yn-1-ylamino)benzenesulfonamide

Into a 8 mL sealed tube was placed 4-amino-3-methoxy-N,N-dimethylbenzenesulfonamide (0.151 g, 655.71 μmol), 3-bromoprop-1-yne (0.102 g, 857.43 μmol), K₂CO₃ (0.263 g, 1.90 mmol), NMP (2 mL). The reaction mixture was stirred under nitrogen at 80° C. for 4 h. The reaction was quenched with water (5 mL). The resulted solution was extracted with EA (3×20 mL), washed with brine (20 mL), dried over anhydrous sodium sulfate, filtered and concentrated under vacuum. The crude product was purified by silica gel column eluted with EA/hexane (v/v=2/3). This resulted in 0.119 g (443.48 μmol, 67.63% yield) of 3-methoxy-N,N-dimethyl-4-(prop-2-yn-1-ylamino)benzenesulfonamide as light yellow solid. LCMS: m/z=269 [M+1]⁺

### Step 4 3-methoxy-N,N-dimethyl-4-((3-(7-((1-methylpiperidin-4-yl)amino)-3-(2,2,2-trifluoroethyl)benzo[b]thiophen-2-yl)prop-2-yn-1-yl)amino)benzenesulfonamide (21)

Into a 10-mL round-bottom flask was placed 3-methoxy-N,N-dimethyl-4-(prop-2-yn-1-ylamino)benzenesulfonamide (0.098 g, 365.22 μmol), N-(2-iodo-3-(2,2,2-trifluoroethyl)benzo[b]thiophen-7-yl)-1-methylpiperidin-4-amine (0.058 g, 127.67 μmol), Pd(PPh₃)₂Cl₂ (0.036 g, 50.99 μmol), CuI (0.019 g, 99.76 μmol), DIEA (0.044 g, 340.44 μmol), methyl sulfoxide (1 mL). The reaction was stirred under nitrogen atmosphere at RT for 1 h. The reaction was quenched with water (5 mL). The resulted solution was extracted with EA (3×20 mL), washed with brine (20 mL), dried over anhydrous sodium sulfate, filtered and concentrated under vacuum. The crude product was purified by preparative HPLC (Mobile Phase A: water (0.1% ammonium hydroxide), Mobile Phase B: ACN; Flow rate: 70 mL/min; Gradient: 50-80-95-95% B (2-30-45-60 min); 220 nm; RT: 41.58-49.20) to provide the desired product. This resulted in 0.035 g (58.85 μmol, 40.09% yield) of 3-methoxy-N,N-dimethyl-4-((3-(7-((1-methylpiperidin-4-yl)amino)-3-(2,2,2-trifluoroethyl)benzo[b]thiophen-2-yl)prop-2-yn-1-yl)amino)benzenesulfonamide (21) as white solid. LCMS: m/z=595 [M+1]⁺.

¹H NMR (400 MHz, DMSO-d₆) δ 7.28-7.22 (m, 2H), 7.32-7.10 (m, 2H), 6.91 (d, J=8.4 Hz, 1H), 6.67 (d, J=7.8 Hz, 1H), 6.49 (t, J=6.2 Hz, 1H), 5.92 (s, 1H), 5.78 (s, 1H), 5.16 (d, J=7.9 Hz, 1H), 4.80 (d, J=49.3 Hz, 2H), 3.83 (d, J=35.5 Hz, 5H), 2.79 (d, J=11.1 Hz, 2H), 2.56 (s, 6H), 2.20 (s, 3H), 2.08-2.00 (m, 2H), 1.92-1.84 (m, 2H), 1.60-1.53 (m, 2H), 1.23 (s, 1H).

### Example 22

### N-(2-(3-((2-methoxy-4-(morpholinosulfonyl)phenyl)amino)prop-1-yn-1-yl)-3-(2,2,2-trifluoroethyl)benzo[b]thiophen-7-yl)-1-methylpiperidin-4-amine (22)

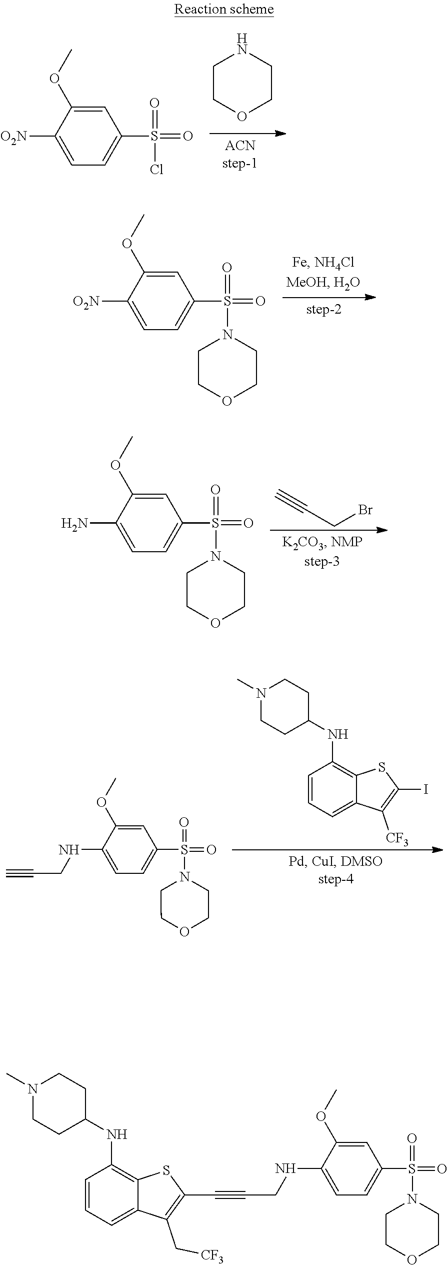

### Experimental Details

### Step 1 4-((3-methoxy-4-nitrophenyl)sulfonyl)morpholine

Into a 20 mL sealed tube was placed 3-methoxy-4-nitrobenzenesulfonyl chloride (0.529 g, 2.10 mmol), morpholine (0.387 g, 4.44 mmol), ACN (5 mL). The reaction was stirred at RT for 0.5 h. The reaction was stirred at RT for 0.5 h. The reaction mixture was purified with silica gel column eluted with EA/hexane (v/v=2/3). This resulted in 0.690 g (2.28 mmol, 100.00% yield) of 4-((3-methoxy-4-nitrophenyl)sulfonyl)morpholine as faint yellow solid. LCMS: m/z=303 [M+1]⁺.

### Step 2 2-methoxy-4-(morpholinosulfonyl)aniline

Into a 40 mL sealed tube was placed 4-((3-methoxy-4-nitrophenyl)sulfonyl)morpholine (0.669 g, 2.57 mmol), iron (1.275 g, 22.83 mmol), NH₄Cl (1.220 g, 22.80 mmol), EtOH (10 mL), water (2 mL). The reaction mixture was stirred under nitrogen at 90° C. for 16 h. The reaction mixture was filtered through celite pad and the filter cake was washed with methanol (2×20 mL). The filterate was then concentrated under reduced pressure to afford the product. This resulted in 0.392 g (1.43 mmol, 56.00% yield) of 2-methoxy-4-(morpholinosulfonyl)aniline as faint yellow solid. LCMS: m/z=273 [M+1]⁺.

### Step 3 2-methoxy-4-(morpholinosulfonyl)-N-(prop-2-yn-1-yl)aniline

Into a 8 mL sealed tube was placed 2-methoxy-4-(morpholinosulfonyl)aniline (0.161 g, 591.21 μmol), 3-bromoprop-1-yne (0.099 g, 832.21 μmol), K₂CO₃ (0.251 g, 1.81 mmol), NMP (2 mL). The reaction mixture w as stirred under nitrogen at 80° C. for 16 h. The reaction was quenched with water (5 mL). The resulted solution was extracted with EA (3×20 mL), washed with brine (20 mL), dried over anhydrous sodium sulfate, filtered and concentrated under vacuum. The crude product was purified with silica gel column eluted with EA/hexane (v/v=2/3). This resulted in 0.138 g (444.63 μmol, 75.20% yield) of 2-methoxy-4-(morpholinosulfonyl)-N-(prop-2-yn-1-yl)aniline as faint yellow solid. LCMS: m/z=311 [M+1]⁺.

### Step 4 N-(2-(3-((2-methoxy-4-(morpholinosulfonyl)phenyl)amino)prop-1-yn-1-yl)-3-(2,2,2-trifluoroethyl)benzo[b]thiophen-7-yl)-1-methylpiperidin-4-amine (22)

Into a 10-mL round-bottom flask was placed 2-methoxy-4-(morpholinosulfonyl)-N-(prop-2-yn-1-yl)aniline (0.072 g, 231.98 μmol), N-(2-iodo-3-(2,2,2-trifluoroethyl)benzo[b]thiophen-7-yl)-1-methylpiperidin-4-amine (0.128 g, 281.75 μmol), Pd(PPh₃)₂Cl₂ (0.036 g, 50.99 μmol), CuI (0.013 g, 68.25 μmol), DIEA (0.072 g, 557.09 μmol), methyl sulfoxide (2 mL). The reaction was stirred under nitrogen atmosphere at RT for 2 h. The reaction was quenched with water (5 mL). The resulted solution was extracted with EA (3×20 mL), washed with brine (20 mL), dried over anhydrous sodium sulfate, filtered and concentrated under vacuum. The crude product was purified by preparative HPLC (Mobile Phase A: water (0.1% ammonium hydroxide), Mobile Phase B: ACN; Flow rate: 40 mL/min; Gradient: 40-80-80% B (2-30-60 min); 220 nm; RT: 24.95-29.32) to provide the desired product. This resulted in 0.035 g (58.85 μmol, 40.09% yield) of N-(2-(3-((2-meth oxy-4-(morpholinosulfonyl)phenyl)amino)prop-1-yn-1-yl)-3-(2,2,2-trifluoroethyl)benzo[b]thiophen-7-yl)-1-methylpiperidin-4-amine (22) as white solid. LCMS: m/z=595 [M+1]⁺.

¹H NMR (400 MHz, DMSO-d₆) δ 7.29-7.21 (m, 2H), 7.14 (d, J=8.0 Hz, 1H), 7.04 (d, J=1.9 Hz, 1H), 6.92 (d, J=8.4 Hz, 1H), 6.66 (d, J=7.8 Hz, 1H), 6.55 (t, J=6.2 Hz, 1H), 5.31 (d, J=7.9 Hz, 1H), 4.39 (d, J=6.2 Hz, 2H), 3.84 (d, J=39.1 Hz, 5H), 3.62 (t, J=4.5 Hz, 4H), 2.83 (t, J=4.8 Hz, 4H), 2.78-2.70 (m, 2H), 2.17 (s, 3H), 1.99-1.89 (m, 2H), 1.93-1.83 (m, 2H), 1.56-1.49 (m, 2H), 1.23 (s, 1H).

### Example 23

### 3-methoxy-4-((3-(7-((1-methylpiperidin-4-yl)amino)-3-(2,2,2-trifluoroethyl)benzo[b]thiophen-2-yl)prop-2-yn-1-yl)amino)benzenesulfonamide (23)

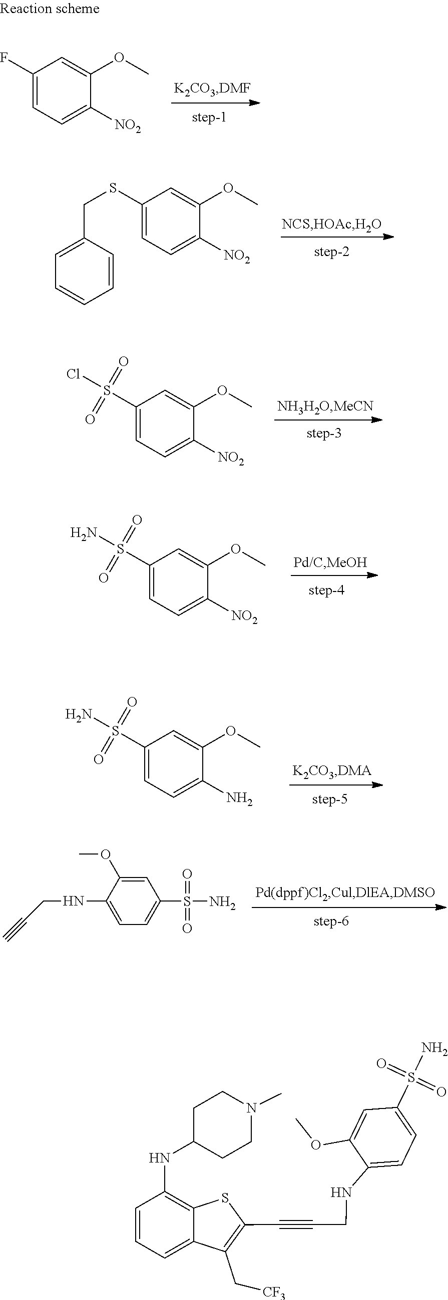

### Experimental Details

### Step 1. benzyl(3-methoxy-4-nitrophenyl)sulfane

To a mixture of 4-fluoro-2-methoxy-1-nitrobenzene (20.50 g, 119.80 mmol) and K₂CO₃ (125.57 g, 908.57 mol) in 200 mL DMF was added phenylmethanethiol (16.34 g, 131.77 mol) at 0° C. The mixture was stirred at room temperature for 8 hours and the mixture was added to 500 mL water, the mixture was filtered and washed with water (100 mL×2) and dried in the oven. This resulted in 18.10 g (54.88%) of benzyl (3-methoxy-4-nitrophenyl)sulfane as little yellow solid. LCMS: m/z=276 [M+1]⁺

### Step 2. 3-methoxy-4-nitrobenzenesulfonyl chloride

Into a 100-mL round-bottom flask was placed benzyl(3-methoxy-4-nitrophenyl)sulfane (14.47 g, 52.56 mmol) 5, HOAc (90 mL), H₂O (15 mL). The mixture was cooled to 0° C. Then NCS (30.45 g, 228.03 mmol) was added over 5 min maintaining the internal temperature below 5° C. The mixture was stirred for 1 h at RT. The reaction mixture was poured into water (150 mL) and extracted with EA (300 mL×2). The combined organic layers was washed with brine (200 mL), dried over anhydrous sodium sulfate, filtered, and concentrated under reduced pressure. The residue was purified by silica gel column, eluted with EA/hexane (v/v=1/1) to afford 3-methoxy-4-nitrobenzenesulfonyl chloride (9.792 g, 74.04% yield) as a little yellow solid.

### Step 3. 3-methoxy-4-nitrobenzenesulfonamide

To a mixture of 3-methoxy-4-nitrobenzenesulfonyl chloride (0.510 g, 2.03 mmol) in 10 mL MeCN was added ammonium hydroxide (1 mL) at 0° C. The mixture was stirred at room temperature for 0.5 h. The mixture was concentrated under reduced pressure. This resulted in 3-methoxy-4-nitrobenzenesulfonamide (0.491 g, crude) as off-white solid. LCMS: m/z=233 [M+1]⁺

### Step 4. 4-amino-3-methoxybenzenesulfonamide

To a solution of 3-methoxy-4-nitrobenzenesulfonamide (0.481 g, 2.07 mmol) in MeOH (8 mL) was added Pd/C (10%, 0.097 g) under N₂. The mixture was degassed under vacuum and purged with H₂(g) for three times. The reaction mixture was stirred at room temperature for 2 h. The solid was filtered off and the fil ter cake was washed with MeOH (10 mL). The filtrate was concentrated under vacuum to afford 4-amino-3-methoxybenzenesulfonamide (0.470 g, crude) as grey solid. LCMS: m/z=203 [M+1]⁺

### Step 5. 3-methoxy-4-(prop-2-yn-1-ylamino)benzenesulfonamide

A mixture of 4-amino-3-methoxybenzenesulfonamide (0.465 g, 2.30 mmol), 3-bromoprop-1-yne (0.595 g, 5.00 mmol), and K₂CO₃ (0.958 g, 6.93 mmol) in DMA (10 mL) was degassed and purged with N₂(g) for three times. The mixture was stirred at 50° C. for 12 h. The reaction mixture was quenched by the addition of water (20 mL) and extracting with EA (100 mL×3). The combined organic layers was washed with brine (30 mL×4), filtered, and concentrated under reduced pressure. The residue was purified with C18 column eluted with ACN/water (v/v=1/2) to afford 0.152 g (27.51%) of 3-methoxy-4-(prop-2-yn-1-ylamino) benzenesulfonamide as yellow solid. LCMS: m/z=241 [M+1]⁺

### Step 6. 3-methoxy-4-((3-(7-((1-methylpiperidin-4-yl)amino)-3-(2,2,2-trifluoroethyl)benzo[b]thiophen-2-yl)prop-2-yn-1-yl)amino)benzenesulfonamide (23)

Into a 8-mL sealed tube purged and maintained with an inert atmosphere of nitrogen, was placed N-(2-iodo-3-(2,2,2-trifluoroethyl)benzo[b]thiophen-7-yl)-1-methylpiperidin-4-amine (0.051 g, 0.11 mmol), 3-methoxy-4-(prop-2-yn-1-ylamino)benzenesulfonamide (0.048 g, 0.20 mmol), Pd(dppf)Cl₂ (0.024 g, 0.04 mmol), CuI (0.018 g, 0.09 mmol), DIEA (0.057 g, 0.44 mmol), DMSO (1 mL). The reaction mixture was stirred at room temperature for 1 h. The mixture was quenched with extracted with H₂O (20 mL), extracted with EA (20 mL×2). The combined organic layers were washed with brine (10 mL), separated and concentrated under vacuum. The mixture was purified by preparative HPLC (Mobile Phase A: water (10 mmoL/L ammonium hydroxide), Mobile Phase B: ACN; Flow rate: 70 mL/min; Gradient: 50-85-95% B (2-30-40 min); 220 nm; RT: 32.686-34.791 min) to afford 3-methoxy-4-((3-(7-((1-methylpiperidin-4-yl)amino)-3-(2,2,2-trifluoroethyl)benzo[b]thiophen-2-yl)prop-2-yn-1-yl)amino)benzenesulfonamide (23) (0.024 g, 37.73% yield) as white solid. LCMS: m/z=567 [M+1]⁺

¹H NMR (400 MHz, MeOD) δ 7.50-7.42 (m, 1H), 7.32 (t, J=5.9 Hz, 1H), 7.26 (t, J=7.9 Hz, 1H), 7.12 (d, J=8.0 Hz, 1H), 6.86 (d, J=8.4 Hz, 1H), 6.71 (t, J=7.7 Hz, 1H), 4.37 (s, 2H), 3.93 (s, 3H), 3.75-3.59 (m, 2H), 3.58-3.45 (m, 1H), 2.90 (d, J=12.0 Hz, 2H), 2.32 (d, J=6.0 Hz, 3H), 2.22 (t, J=11.4 Hz, 2H), 2.06 (d, J=12.0 Hz, 2H), 1.67-1.53 (m, 2H).

### Example 24

### N-(2,3-dihydroxypropyl)-3-methoxy-4-((3-(7-((1-methylpiperidin-4-yl)amino)-3-(2,2,2-trifluoroethyl)benzol[b]thiophen-2-yl)prop-2-yn-1-yl)amino)benzenesulfonamide (24)

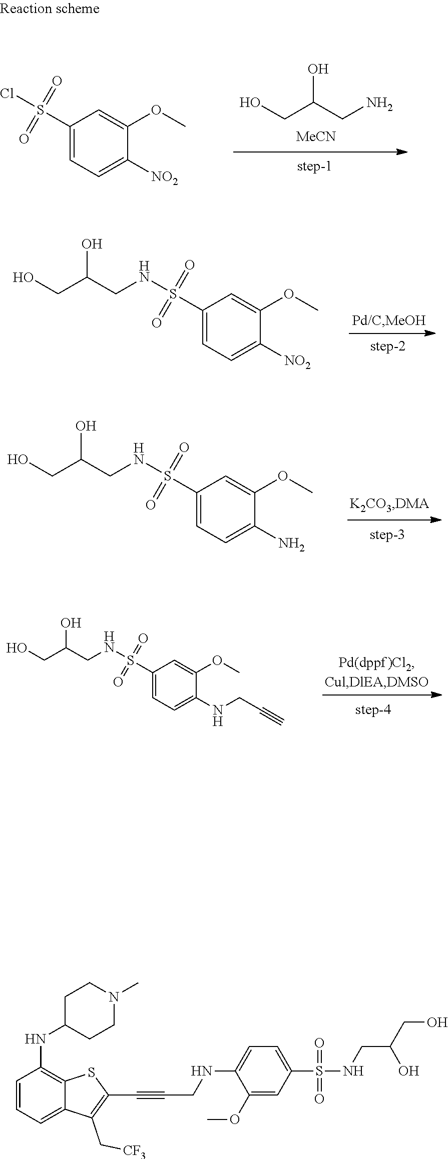

### Experimental Details

### Step 1. benzyl(3-methoxy-4-nitrophenyl)sulfane

To a mixture of 3-methoxy-4-nitrobenzenesulfonyl chloride (0.512 g, 2.03 mmol) in 10 mL MeCN was added 3-aminopropane-1,2-diol (0.238 g, 2.61 mmol) at 0° C. The mixture was stirred at room temperature for 3 h. The resulted mixture was concentrated under reduced pressure. This resulted in N-(2,3-dihydroxypropyl)-3-methoxy-4-nitrobenzenesulfonamide (1.033 g, crude) as colorless oil. LCMS: m/z=307 [M+1]⁺

### Step 2. 4-amino-N-(2,3-dihydroxypropyl)-3-methoxybenzenesulfonamide

To a solution of N-(2,3-dihydroxypropyl)-3-methoxy-4-nitrobenzenesulfonamide (1.031 g, 3.36 mmol) in MeOH (8 mL) was added Pd/C (10%, 0.219 g) under N₂(g). The mixture was degassed under vacuum and p urged with H₂(g) for three times. The reaction mixture was stirred at room temperature for 2 hours. The solid was filtered off and the filter cake was washed with MeOH (10 mL). The combined filtrate was concentrated under vacuum to give 4-amino-N-(2,3-dihydroxypropyl)-3-methoxybenzenesulfonamide (1.004 g, cm de) as grey solid. LCMS: m/z=277 [M+1]⁺

### Step 3. N-(2,3-dihydroxypropyl)-3-methoxy-4-(prop-2-yn-1-ylamino)benzenesulfonamide

A mixture of 4-amino-N-(2,3-dihydroxypropyl)-3-methoxybenzenesulfonamide (1.001 g, crude), 3-bromoprop-1-yne (0.467 g, 3.93 mmol), and K₂CO₃ (0.795 g, 5.75 mmol) in NMP (10 mL) was degassed and purged with N₂(g) for three times. The mixture was then stirred at 80° C. for 48 h. The reaction mixture was quenched by the addition of water (20 mL), extracted with EA (100 mL×3). The combined organic layers w as washed with brine (30 mL×4), filtered and concentrated under reduced pressure. The residue was purified by C18 column eluted with ACN/water (v/v=1/4) to afford 0.078 g (6.85%) of N-(2,3-dihydroxypropyl)-3-methoxy-4-(prop-2-yn-1-ylamino)benzenesulfonamide as colorless oil. LCMS: m/z=315 [M+1]⁺

### Step 4. N-(2,3-dihydroxypropyl)-3-methoxy-4-((3-(7-((1-methylpiperidin-4-yl)amino)-3-(2,2,2-trifluoroethyl)benzo[b]thiophen-2-yl)prop-2-yn-1-yl)amino)benzenesulfonamide (24)

Into a 8-mL sealed tube purged and maintained with an inert atmosphere of nitrogen, was placed N-(2-iodo-3-(2,2,2-trifluoroethyl)benzo[b]thiophen-7-yl)-1-methylpiperidin-4-amine (0.048 g, 0.11 mmol), N-(2,3-dihydroxypropyl)-3-methoxy-4-(prop-2-yn-1-ylamino)benzenesulfonamide (0.077 g, 0.24 mmol), Pd(dppf)Cl₂ (0.019 g, 0.03 mmol), CuI (0.009 g, 0.05 mmol), DIEA (0.062 g, 0.48 mmol), DMSO (1 mL). The reaction mixture was stirred at room temperature for 1 h. The reaction mixture was quenched by the addition of water (20 mL), extracted with EA (20 mL×2). The combined organic layers was washed with brine (10 mL), separated and concentrated under vacuum. The mixture was purified by preparative HPLC (Mobile Phase A: water (10 mmoL/L ammonium hydroxide), Mobile Phase B: MeOH; Flow rate: 40 mL/min; Gradient: 35-65-95% B (2-30-60 min); 270 nm; RT: 38.125-42.751 min) to afford N-(2,3-dihydroxypropyl)-3-methoxy-4-((3-(7-((1-methylpiperidin-4-yl)amino)-3-(2,2,2-trifluoroethyl)benzo[b]thiophen-2-yl)prop-2-yn-1-yl)amino)benzenesulfonamide (24) (0.015 g, 22.14% yield) as white solid. LCMS: m/z=641 [M+1]⁺

¹H NMR (400 MHz, DMSO) δ 7.32-7.23 (m, 2H), 7.21 (s, 1H), 7.18-7.10 (m, 1H), 7.11-7.06 (m, 1H), 6.83 (d, J=8.0 Hz, 1H), 6.66 (d, J=7.6 Hz, 1H), 6.33 (t, J=6.0 Hz, 1H), 5.32 (d, J=7.6 Hz, 1H), 4.72 (s, 1H), 4.51 (s, 1H), 4.36 (d, J=6.0 Hz, 2H), 3.88-3.75 (m, 4H), 3.55-3.42 (m, 2H), 2.85-2.75 (m, 3H), 2.59-2.52 (m, 1H), 2.20 (s, 3H), 2.12-1.93 (m, 3H), 1.89 (d, J=12.4 Hz, 2H), 1.61-1.47 (m, 2H), 1.37-1.26 (m, 2H).

### Example 25

### N-(2-(3-((2-methoxy-4-(methylsulfonyl)phenyl)amino)prop-1-yn-1-yl)-3-(2,2,2-trifluoroethyl)benzo[b]thiophen-7-yl)-3-methyl-3-azabicyclo[3.2.1]octan-8-amine (25)

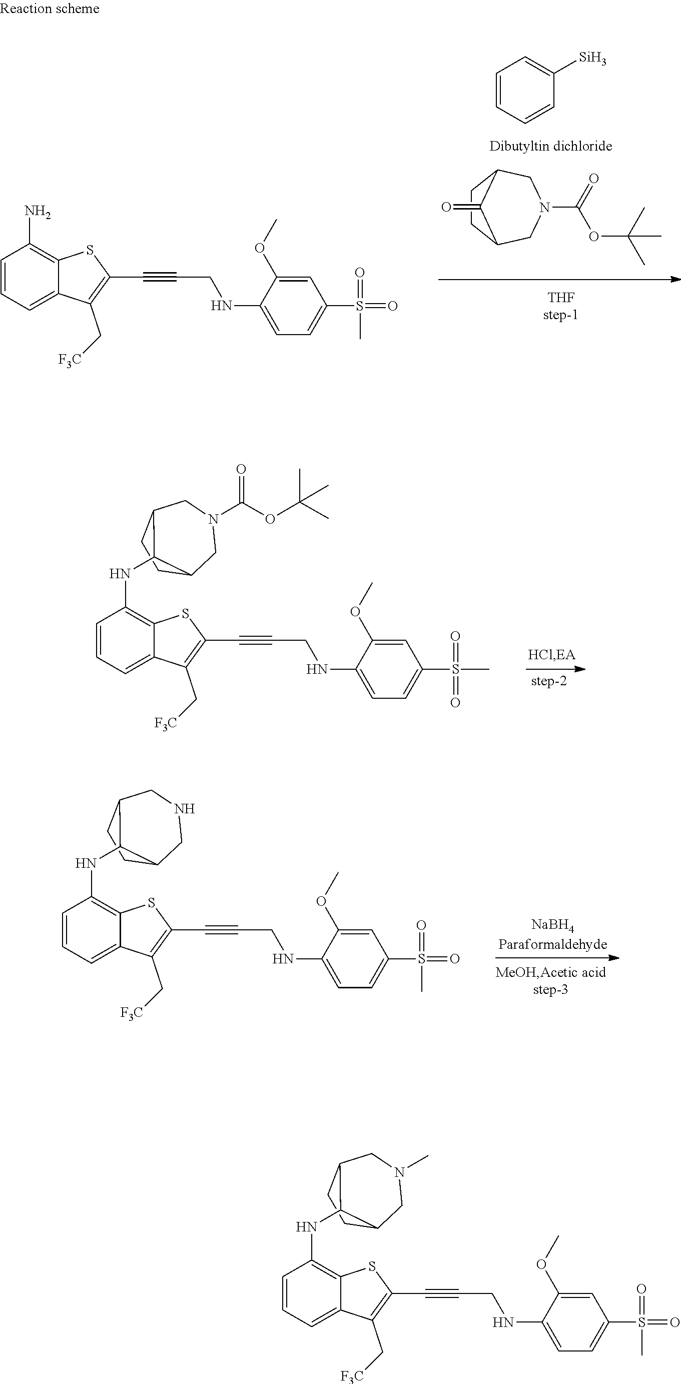

### Experimental Details

### Step 1 tert-butyl 8-((2-(3-((2-methoxy-4-(methylsulfonyl)phenyl)amino)prop-1-yn-1-yl)-3-(2,2,2-trifluoroethyl)benzo[b]thiophen-7-yl)amino)-3-azabicyclo[3.2.1]octane-3-carboxylate

Into a 20 mL sealed tube was placed 2-(3-((2-methoxy-4-(methylsulfonyl)phenyl)amino)prop-1-yn-1-yl)-3-(2,2,2-trifluoroethyl)benzo[b]thiophen-7-amine (0.117 g, 414.07 μmol), tert-butyl 8-oxo-3-azabicyclo[3.2.1]octane-3-carboxylate (0.561 g, 519.34 μmol), dibutyltin dichloride (0.082 g, 269.87 μmol). The reaction was stirred under nitrogen atmosphere at RT for 1 h. Then phenylsilane (0.235 g, 2.17 mmol) was added. The reaction was stirred under nitrogen atmosphere at RT for 48 h. The reaction mixture was concentrated under r educed pressure to afford crude product. The crude product was purified by silica gel column eluted with EA/hexane (v/v=3/2). This resulted in 0.194 g (286.22 μmol, 69.12% yield) of tert-butyl 8-((2-(3-((2-methoxy-4-(methylsulfonyl)phenyl)amino)prop-1-yn-1-yl)-3-(2,2,2-trifluoroethyl)benzo[b]thiophen-7-yl)amino)-3-azabicyclo[3.2.1]octane-3-carboxylate as yellow solid. LCMS: m/z=678 [M+1]⁺.

### Step 2 N-(2-(3-((2-methoxy-4-(methylsulfonyl)phenyl)amino)prop-1-yn-1-yl)-3-(2,2,2-trifluoroethyl)benzo[b]thiophen-7-yl)-3-azabicyclo[3.2.1]octan-8-amine

Into a 8 mL sealed tube was placed tert-butyl 8-((2-(3-((2-methoxy-4-(methylsulfonyl)phenyl)amino)prop-1-yn-1-yl)-3-(2,2,2-trifluoroethyl)benzo[b]thiophen-7-yl)amino)-3-azabicyclo[3.2.1]octane-3-carboxylate (0.192 g, 283.27 μmol), HCl(g) in EA (2 mL, 4N), EA (2 mL). The reaction was stirred at RT for 1 h. The reaction was concentrated under vacuum. Water (2 mL) was added to the reaction and NaOH (aq, 3N) was added until PH=7. Extracted with EA (3×10 mL), washed with brine (10 mL), dried over anhydrous sodium sulfate, filtered and concentrated under vacuum. This resulted in 0.153 g (264.85 μmol, 93.49% yield) of N-(2-(3-((2-methoxy-4-(methylsulfonyl)phenyl)amino)prop-1-yn-1-yl)-3-(2,2,2-trifluoroethyl)benzo[b]thiophen-7-yl)-3-azabicyclo[3.2.1]octan-8-amine as brown solid. The reaction mixture was purified by C18 column eluted with ACN/H₂O (v/v=1/1). LCMS: m/z=578 [M+1]⁺.

### Step 3 N-(2-(3-((2-methoxy-4-(methylsulfonyl)phenyl)amino)prop-1-yn-1-yl)-3-(2,2,2-trifluoroethyl)benzo[b]thiophen-7-yl)-3-methyl-3-azabicyclo[3.2.1]octan-8-amine (25)

Into a 8 mL sealed tube was placed N-(2-(3-((2-methoxy-4-(methylsulfonyl)phenyl)amino)prop-1-yn-1-yl)-3-(2,2,2-trifluoroethyl)benzo[b]thiophen-7-yl)-3-azabicyclo[3.2.1]octan-8-amine (0.152 g, 263.12 μmol), paraformaldehyde (0.054 g, 1.79 mmol), acetic acid (0.3 mL), methanol (2 mL). The reaction mixture was stirred at RT for 1 h. Then NaBH₃CN (0.090 g, 2.37 mmol) was added. The reaction mixture was stirred at RT for 4 h. The reaction was quenched with water (10 mL), the resulted solution was extracted with EA (3×20 mL), washed with brine (20 mL), dried over anhydrous sodium sulfate, filtered and concentrated under vacuum. The crude product was purified by preparative HPLC (Mobile Phase A: water (0.1% ammonium hydroxide), Mobile Phase B: ACN; Flow rate: 70 mL/min; Gradient: 50-80-100% B (2-30-60 min); 270 nm; RT: 44.33-46.51) to provide the desired product. This resulted in 0.023 g (38.87 μmol, 14.77% yield) of N-(2-(3-((2-methoxy-4-(methylsulfonyl)phenyl)amino)prop-1-yn-1-yl)-3-(2,2,2-trifluoroethyl)benzo[b]thiophen-7-yl)-3-methyl-3-azabicyclo[3.2.1]octan-8-amine (25) as white solid. LCMS: m/z=592 [M+1]⁺.

¹H NMR (400 MHz, DMSO-d₆) δ 7.42-7.32 (m, 1H), 7.32-7.25 (m, 2H), 7.20 (d, J=7.9 Hz, 1H), 6.89 (d, J=8.4 Hz, 1H), 6.71 (d, J=7.7 Hz, 1H), 6.52 (t, J=6.2 Hz, 1H), 5.18 (d, J=3.6 Hz, 1H), 4.39 (d, J=6.2 Hz, 2H), 3.86 (d, J=33.0 Hz, 5H), 3.51-3.42 (m, 1H), 3.10 (s, 3H), 2.43 (d, J=10.5 Hz, 2H), 2.32-3.28 (m, 4H), 2.13 (s, 3H), 1.72 (d, J=3.6 Hz, 4H).

### Example 26

### N-(2-(3-((2-methoxy-4-(methylsulfonyl)phenyl)amino)prop-1-yn-1-yl)-3-(2,2,2-trifluoroethyl)benzo[b]thiophen-7-yl)-8-methyl-8-azabicyclo[3.2.1]octan-3-amine (26)

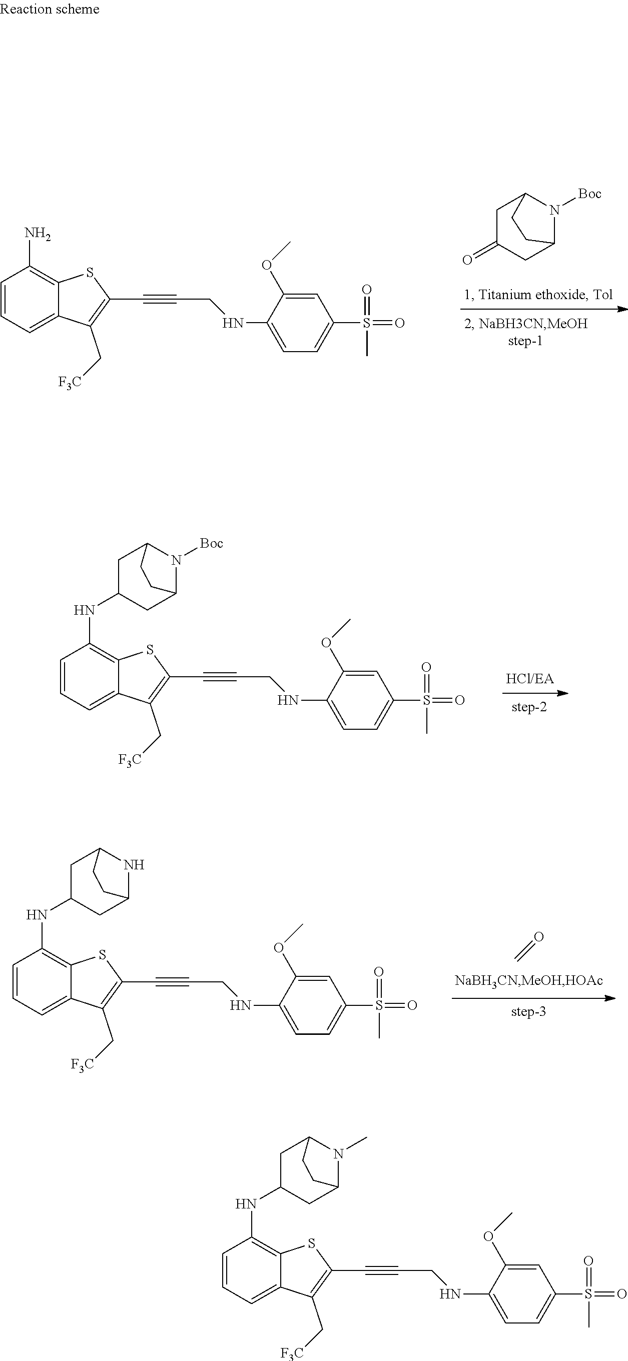

### Experimental Details

### Step 1. tert-butyl 3-((2-(3-((2-methoxy-4-(methylsulfonyl)phenyl)amino)prop-1-yn-1-yl)-3-(2,2,2-trifluoroethyl) benzo[b]thiophen-7-yl)amino)-8-azabicyclo[3.2.1]octane-8-carboxylate

Into a 20 mL vial was placed 2-(3-((2-methoxy-4-(methylsulfonyl)phenyl)amino)prop-1-yn-1-yl)-3-(2,2,2-trifluoroethyl)benzo[b]thiophen-7-amine (0.147 g, 313.76 μmol), tert-butyl 3-oxo-8-azabicyclo[3.2.1]octane-8-carboxylate (0.442 g, 1.96 mmol), titanium ethoxide (0.498 g, 2.18 mmol), toluene (3 mL). The reaction was stirred at 110° C. for 16 h. The reaction was concentrated under vacuum. The crude was added MeOH (3 mL) and Sodium cyanoboronhydride (0.159 g, 3.71 mmol). The reaction stirred overnight at RT. The reaction was quenched with water (50 mL), extracted with EA (3×20 mL). The organic layers combined, washed with brine (50 mL), dried over anhydrous sodium sulfate, filtered and concentrated under vacuum. The crude was purified by silica gel column, eluted with EA/hexane (v/v=1/2). This resulted in 0.330 g (crude) of tert-butyl 3-((2-(3-((2-methoxy-4-(methylsulfonyl)phenyl)amino)prop-1-yn-1-yl)-3-(2,2,2-trifluoroethyl)benzo[b]thiophen-7-yl)amino)-8-azabicyclo[3.2.1]octane-8-carboxylate as yellow oil. LCMS: m/z=678 [M+1]⁺.

### Step 2. N-(2-(3-((2-methoxy-4-(methylsulfonyl)phenyl)amino)prop-1-yn-1-yl)-3-(2,2,2-trifluoroethyl)benzo[b]thiophen-7-yl)-8-azabicyclo[3.2.1]octan-3-amine

Into a 50 mL flask was placed tert-butyl 3-((2-(3-((2-methoxy-4-(methylsulfonyl)phenyl)amino)prop-1-yn-1-yl)-3-(2,2,2-trifluoroethyl)benzo[b]thiophen-7-yl)amino)-8-azabicyclo[3.2.1]octane-8-carboxylate (0.330 g, 486.87 μmol), HCl in EA (20 mL, 4.0 M). The reaction stirred at RT for 3 h. The reaction mixture was adjusted to PH=8 by KHCO₃ (aq.) at 0° C. The mixture was extracted with EA (3×20 mL). The organic layers were combined, washed with brine (50 mL), dried over anhydrous sodium sulfate, filtered and concentrated under vacuum. The crude was purified by silica gel column, eluted with MeOH/DCM (v/v=1/9). This resulted in 0.074 g (26.31% yield) of N-(2-(3-((2-methoxy-4-(methylsulfonyl)phenyl)amino)prop-1-yn-1-yl)-3-(2,2,2-trifluoroethyl)benzo[b]thiophen-7-yl)-8-azabicyclo[3.2.1]octan-3-amine as yellow solid. LCMS: m/z=578 [M+1]⁺.

### Step 3. N-(2-(3-((2-methoxy-4-(methylsulfonyl)phenyl)amino)prop-1-yn-1-yl)-3-(2,2,2-trifluoroethyl)benzo[b]thiophen-7-yl)-8-methyl-8-azabicyclo[3.2.1]octan-3-amine (26)

Into a 8 mL vial was placed N-[2-[3-(2-methoxy-4-methylsulfonyl-anilino)prop-1-ynyl]-3-(2,2,2-trifluoroethyl) benzothiophen-7-yl]-8-azabicyclo[3.2.1]octan-3-amine (0.075 g, 129.82 μmol), polyoxymethylene (0.008 g, 266.44 μmol), sodium cyanoboronhydride (0.058 g, 1.35 mmol), MeOH (3 mL), HOAc (0.1 mL). The reaction mixture was stirred at RT overnight. The reaction was quenched with water (10 mL), extracted with EA (20 mL), washed with brine (20 mL), dried over anhydrous sodium sulfate, filtered and concentrated under vacuum. The mixture was purified by preparative HPLC (Mobile Phase A: water (10 mmoL/L TFA), Mobile Phase B: ACN; Flow rate: 70 mL/min; Gradient: 50-100-100% B (0-45-50 min); 271 nm; RT: 43.810-47.680 min) to provide the desired product. This resulted in 0.017 g (22.13% yield) of N-(2-(3-((2-methoxy-4-(methylsulfonyl)phenyl)amino)prop-1-yn-1-yl)-3-(2,2,2-trifluoroethyl)benzo[b]thiophen-7-yl)-8-methyl-8-azabicyclo[3.2.1]octan-3-amine (26) as off-white solid. LCMS: m/z=592 [M+1]⁺.

¹H NMR (400 MHz, Methanol-d₄) δ 7.52-7.45 (m, 1H), 7.34-7.25 (m, 2H), 7.18 (d, J=8.0 Hz, 1H), 6.93 (d, J=8.4 Hz, 1H), 6.59 (d, J=7.6 Hz, 1H), 4.40 (s, 2H), 3.95 (s, 3H), 3.82 (d, J=12.0 Hz, 1H), 3.75-3.63 (m, 2H), 3.30 (s, 2H), 3.06 (s, 3H), 2.40 (s, 3H), 2.27-2.08 (m, 6H), 1.97 (d, J=14.8 Hz, 2H).

### Example 27

### N-(2-(3-((2-methoxy-4-(methylsulfonyl)phenyl)amino)prop-1-yn-1-yl)-3-(2,2,2-trifluoroethyl)benzo[b]thiophen-7-yl)-2-methyl-2-azabicyclo[2.2.1]heptan-5-amine (27)

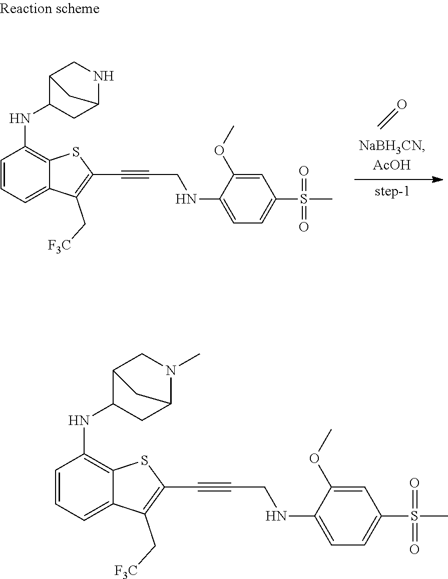

### Experimental Details

### Step 1. N-(2-(3-((2-methoxy-4-(methylsulfonyl)phenyl)amino)prop-1-yn-1-yl)-3-(2,2,2-trifluoroethyl)benzo[b]thiophen-7-yl)-2-methyl-2-azabicyclo[2.2.1]heptan-5-amine (27)

Into a 8 mL reaction vial was placed N-(2-(3-((2-methoxy-4-(methylsulfonyl)phenyl)amino)prop-1-yn-1-yl)-3-(2,2,2-trifluoroethyl)benzo[b]thiophen-7-yl)-2-azabicyclo[2.2.1]heptan-5-amine (0.018 g, 31.93 μmol), paraformaldehyde (0.002 g, 66.60 μmol), acetic acid (0.015 g, 249.78 μmol), methanol (1 mL). The reaction was stirred at rt for 12 h before sodium cyanoboronhydride (0.037 g, 862.95 μmol) was added. The reaction w as stirred at rt for 5 h. The reaction was quenched with aq·Na₂CO₃ (10 mL), extracted with DCM (3×10 mL), concentrated under vacuum. The mixture was purified by preparative HPLC (Mobile Phase A: water (10 mmoL/L ammonium hydroxide), Mobile Phase B: MeOH; Flow rate: 40 mL/min; Gradient: 50-70-90% B (2-30-60 min); 270 nm; RT: 43.971-48.403 min) to afford N-(2-(3-((2-methoxy-4-(methylsulfonyl)phenyl)amino)prop-1-yn-1-yl)-3-(2,2,2-trifluoroethyl)benzo[b]thiophen-7-yl)-2-methyl-2-azabicyclo[2.2.1]heptan-5-amine (27) (0.004 g, 21.68%) as off-white solid. LCMS: m/z=578 [M+1]⁺.

¹H NMR (400 MHz, DMSO) δ 7.39 (d, J=8.3 Hz, 1H), 7.29-7.22 (m, 2H), 7.16 (d, J=7.9 Hz, 1H), 6.89 (d, J=8.4 Hz, 1H), 6.64 (d, J=7.7 Hz, 1H), 6.52 (t, J=6.2 Hz, 1H), 5.53 (d, J=4.5 Hz, 1H), 4.38 (d, J=6.2 Hz, 2H), 3.90 (s, 3H), 3.81 (q, J=10.6 Hz, 2H), 3.12 (d, J=12.6 Hz, 3H), 2.98 (s, 1H), 2.74-2.59 (m, 2H), 2.36-2.27 (m, 1H), 2.22 (d, J=13.6 Hz, 3H), 2.01-1.83 (m, 1H), 1.69 (d, J=8.8 Hz, 1H), 1.62-1.54 (m, 1H), 1.42 (d, J=9.4 Hz, 1H), 1.23 (s, 1H).

### Example 28

### N-(2-(3-((2-methoxy-4-(methylsulfonyl)phenyl)amino)prop-1-yn-1-yl)-3-(2,2,2-trifluoroethyl)benzo[b]thiophen-7-yl)-2-azabicyclo[2.2.1]heptan-5-amine (28)

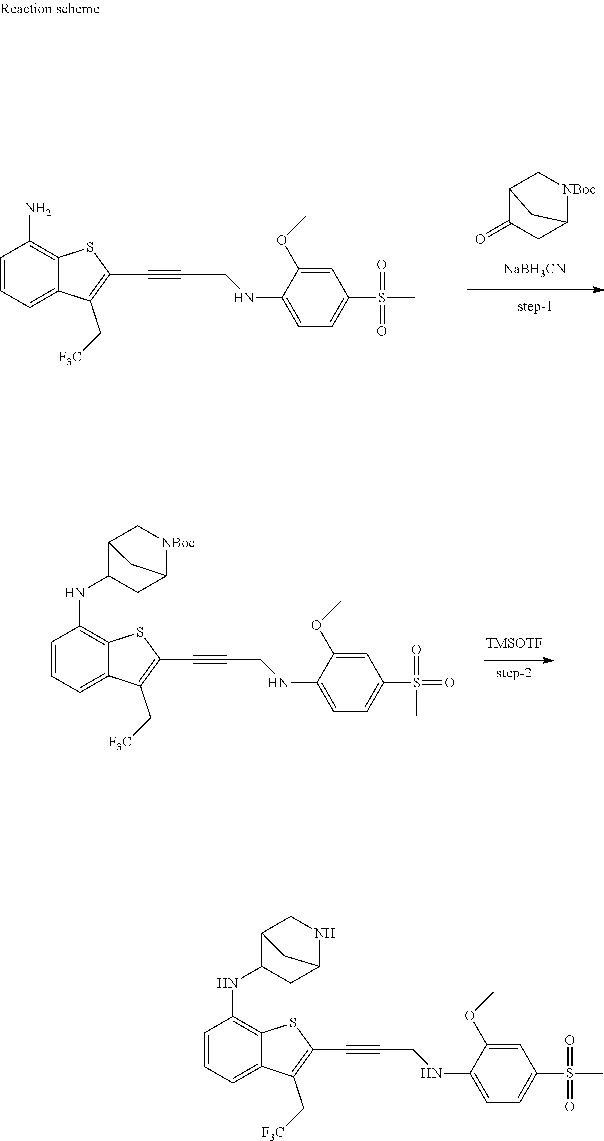

### Experimental Details

### Step 1. tert-butyl 5-((2-(3-((2-methoxy-4-(methylsulfonyl)phenyl)amino)prop-1-yn-1-yl)-3-(2,2,2-trifluoroethyl)benzo[b]thiophen-7-yl)amino)-2-azabicyclo[2.2.1]heptane-2-carboxylate

Into a 8 mL reaction vial was placed 2-(3-((2-methoxy-4-(methylsulfonyl)phenyl)amino)prop-1-yn-1-yl)-3-(2,2,2-trifluoroethyl)benzo[b]thiophen-7-amine (0.061 g, 130.19 μmol), tert-butyl 5-oxo-2-azabicyclo[2.2.1]heptane-2-carboxylate (0.214 g, 1.01 mmol), titanium ethoxide (0.233 g, 1.021 mmol), toluene (2 mL). The reaction stirred at 100° C. for 2 h before cooled to RT. Toluene was concentrated under vacuum. The residue was added MeOH (3 mL) and sodium cyanoboronhydride (0.030 g, 699.69 μmol). The reaction stirred at RT for 2 h. The reaction mixture was quenched by the addition of water (10 mL), extracted with EA (3×30 m L), washed with brine (20 mL), dried over anhydrous sodium sulfate and concentrated under vacuum. The crude was purified by silica gel column eluted with EA/hexane (v/v=1/3) to afford tert-butyl 5-((2-(3-((2-methoxy-4-(methylsulfonyl)phenyl)amino)prop-1-yn-1-yl)-3-(2,2,2-trifluoroethyl)benzo[b]thiophen-7-yl)amino)-2-azabicyclo[2.2.1]heptane-2-carboxylate (0.083 g, 96.03%) as yellow solid. LCMS: m/z=664 [M+1]⁺.

### Step 2. N-(2-(3-((2-methoxy-4-(methylsulfonyl)phenyl)amino)prop-1-yn-1-yl)-3-(2,2,2-trifluoroethyl)benzo[b]thiophen-7-yl)-2-azabicyclo[2.2.1]heptan-5-amine (28)

Into a 8 mL reaction vial was added tert-butyl 5-((2-(3-((2-methoxy-4-(methylsulfonyl)phenyl)amino)prop-1-yn-1-yl)-3-(2,2,2-trifluoroethyl)benzo[b]thiophen-7-yl)amino)-2-azabicyclo[2.2.1]heptane-2-carboxylate (0.080 g, 120.52 μmol), DCM (3 mL), 2,6-Bis(1,1-dimethylethyl)pyridine (0.122 g, 637.70 μmol). The reaction mixture was stirred at 0° C. and trimethylsilyl trifluoromethanesulfonate (0.161 g, 724.38 μmol) was added. The reaction was stirred at rt for 12 h. The reaction was quenched with aq·Na₂CO₃ (10 mL), extracted with DCM (3×10 mL), concentrated under vacuum. The mixture was purified by preparative HPLC (Mobile Phase A: water (10 mmoL/L ammonium hydroxide), Mobile Phase B: ACN; Flow rate: 70 mL/min; Gradient: 45-80-95-95% B (2-30-52-90 min); 270 nm; RT: 61.333-73.370 min) to afford N-(2-(3-((2-methoxy-4-(methylsulfonyl)phenyl)amino)prop-1-yn-1-yl)-3-(2,2,2-trifluoroethyl)benzo[b]thiophen-7-yl)-2-azabicyclo[2.2.1]heptan-5-amine (28) (0.028 g, 41.21%) as off-white solid. LCMS: m/z=564 [M+1]⁺.

¹H NMR (400 MHz, DMSO) δ 7.39 (d, J=8.3 Hz, 1H), 7.30-7.21 (m, 2H), 7.15 (d, J=7.9 Hz, 1H), 6.89 (d, J=8.4 Hz, 1H), 6.66 (d, J=7.7 Hz, 1H), 6.52 (t, J=6.1 Hz, 1H), 5.61 (d, J=6.1 Hz, 1H), 4.38 (d, J=6.1 Hz, 2H), 3.96-3.73 (m, 5H), 3.10 (s, 3H), 2.69-2.61 (m, 1H) 2.14-1.95 (m, 1H), 1.57 (d, J=23.6 Hz, 2H), 1.43-1.32 (m, 2H), 1.30-1.11 (m, 2H).

### Example 29

N-(3-(7-((1-methylpiperidin-4-yl)amino)-3-(2,2,2-trifluoroethyl)benzo[b]thiophen-2-yl)prop-2-yn-1-yl)-1H-indol-7-amine (29)

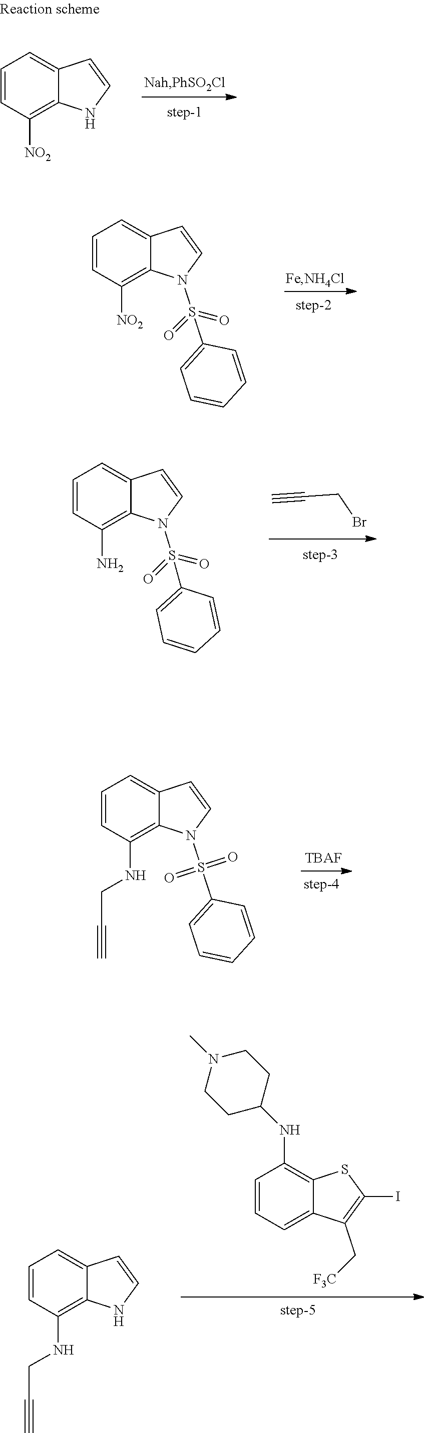

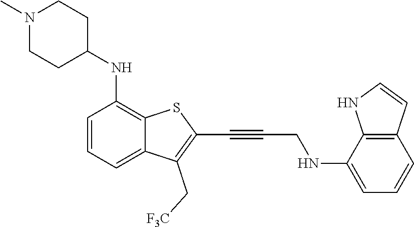 [SMILES: CN1CCC(Nc2cccc3c(CC(F)(F)F)c(C#CCNc4cccc5cc[nH]c45)sc23)CC1]

### Experimental Details

### Step 1. 7-nitro-1-(phenylsulfonyl)-1H-indole

Into a 500 mL flask was placed 7-nitro-1H-indole (5.04 g, 31.08 mmol), tetrahydrofuran (200 mL). Then NaH (4.28 g, 178.35 mmol) was added at 0° C. in portions. The reaction mixture was stirred at 0° C. for 3 h. Then benzenesulfonyl chloride (8.46 g, 47.89 mmol) in tetrahydrofuran (100 mL) was added. The reaction mixture was stirred at 0° C. for another 1 h. The reaction was quenched by water (500 mL), extracted by EA (2×200 mL). The organic layer was combined, washed with brine (200 mL), dried over anhydrous Na₂SO₄ and concentrated under vacuum. The crude was recrystallized with Heptane/EA (v/v=20/1) to give 8.99 g (95.67%) of 7-nitro-1-(phenylsulfonyl)-1H-indole as yellow solid. LCMS: m/z=303 [M+1]⁺.

### Step 2. 1-(phenylsulfonyl)-1H-indol-7-amine

Into a 250 mL flask was placed 7-nitro-1-(phenylsulfonyl)-1H-indole (5.03 g, 16.64 mmol), methanol (80 mL), ammonium chloride (10.19 g, 190.50 mmol) in water (10 mL), iron (4.77 g, 85.42 mmol). The reaction mixture was stirred at 45° C. for 2 h. Then the catalyst was removed by filtration. The filtrate was extracted with EA (2×100 mL). The organic layers was combined, washed with brine (100 mL), dried over anhydrous Na₂SO₄ and concentrated under vacuum. The residue was applied onto a silica gel column eluted with EA/hexane (v/v=2/5). This resulted in 2.84 g (62.68% yield) of 1-(phenylsulfonyl)-1H-indol-7-amine as yellow solid. LCMS: m/z=273 [M+1]⁺.

### Step 3. 1-(phenylsulfonyl)-N-(prop-2-yn-1-yl)-1H-indol-7-amine

Into a 100 mL flask purged and maintained with an inert atmosphere of nitrogen, was placed 1-(phenylsulfonyl)-1H-indol-7-amine (2.002 g, 7.35 mmol), 3-bromopropyne (1.05 g, 8.83 mmol), cesium carbonate (7.313 g, 22.45 mmol), sodium iodide (3.357 g, 22.40 mmol), N,N-dimethylformamide (50 mL). The reaction mixture was stirred at 100° C. for 24 h. The reaction was then quenched by the addition of water (100 mL). The resulting solution was extracted with EA (2×100 mL). The organic layer was combined, washed with brine (100 mL), dried over anhydrous Na₂SO₄ and concentrated under vacuum. The residues were purified by C₁₈ chromatography column eluted with ACN/H₂O (v/v=1/1). This resulted in 1.474 g (64.60% yield) of 1-(phenylsulfonyl)-N-(prop-2-yn-1-yl)-1H-indol-7-amine as yellow solid. LCMS: m/z=311 [M+1]⁺.

### Step 4. N-(prop-2-yn-1-yl)-1H-indol-7-amine

Into a 25 mL flask purged and maintained with an inert atmosphere of nitrogen, was placed 1-(phenylsulfonyl)-N-(prop-2-yn-1-yl)-1H-indol-7-amine (0.500 g, 1.61 mmol), tetrahydrofuran (5 mL), tetrabutylammonium fluoride (5 mL). The reaction mixture was stirred at 80° C. for 3 h. The reaction was then quenched by the addition of ammonium chloride solution (2 M, 30 mL). The resulting solution was extracted with EA (20 mL). The organic layers was combined, washed with brine (20 mL), dried over anhydrous Na₂SO₄ and concentrated under vacuum. The residue was applied onto a silica gel column eluted with EA/hexane (v/v=1/3). This resulted in 0.117 g (42.67% yield) of N-(prop-2-yn-1-yl)-1H-indol-7-amine as brown oil. LCMS: m/z=171 [M+1]⁺.

### Step 5. N-(3-(7-((1-methylpiperidin-4-yl)amino)-3-(2,2,2-trifluoroethyl)benzo[b]thiophen-2-yl)prop-2-yn-1-yl)-1H-indol-7-amine (29)

Into a 25 mL flask purged and maintained with an inert atmosphere of nitrogen, was placed N-(prop-2-yn-1-yl)-1H-indol-7-amine (0.044 g, 258.50 μmol), N-(2-iodo-3-(2,2,2-trifluoroethyl)benzo[b]thiophen-7-yl)-1-methylpiperidin-4-amine (0.099 g, 217.92 μmol), bis(triphenylphosphine)palladium(II) chloride (0.026 g, 36.83 μmol), cuprous iodide (0.009 g, 47.26 μmol), triethylamine (0.038 g, 375.53 μmol), methyl sulfoxide (5 mL). The reaction mixture was stirred at room temperature for 17 h. The reaction was quenched by the addition of water (5 mL) and extracted with EA (2×5 mL). The organic layers was combined, washed with brine (10 mL), dried over anhydrous Na₂SO₄ and concentrated under vacuum. The mixture was purified by preparative HPLC (Mobile Phase A: water (10 mmoL/L ammonium hydroxide), Mobile Phase B: MeOH; Flow rate: 70 mL/min; Gradient: 55-90-100% B (2-30-40 min); 222 nm; RT: 36.225-37.590 min). This resulted in 0.011 g (9.10% yield) of N-(3-(7-((1-methylpiperidin-4-yl)amino)-3-(2,2,2-trifluoroethyl)benzo[b]thiophen-2-yl)prop-2-yn-1-yl)-1H-indol-7-amine (29) as white solid. LCMS: m/z=497 [M+1]⁺.

¹H NMR (400 MHz, DMSO) δ 10.64 (s, 1H), 7.29-7.22 (m, 2H), 7.14 (d, J=7.5 Hz, 1H), 6.93 (d, J=7.6 Hz, 1H), 6.86 (t, J=7.6 Hz, 1H), 6.66 (d, J=7.8 Hz, 1H), 6.47 (d, J=7.6 Hz, 1H), 6.35 (s, 1H), 5.84 (s, 1H), 5.29 (d, J=7.4 Hz, 1H), 4.43 (d, J=5.7 Hz, 2H), 3.86-3.76 (m, 2H), 2.76 (d, J=11.1 Hz, 2H), 2.17 (s, 3H), 1.99 (s, 2H), 1.88 (d, J=13.8 Hz, 2H), 1.53 (d, J=12.5 Hz, 2H), 1.23 (s, 1H).

### Example 30

### N-(3-(7-((1-methylpiperidin-4-yl)amino)-3-(2,2,2-trifluoroethyl)benzo[b]thiophen-2-yl)prop-2-yn-1-yl)-3-(piperidin-4-yl)-1H-indol-7-amine (30)

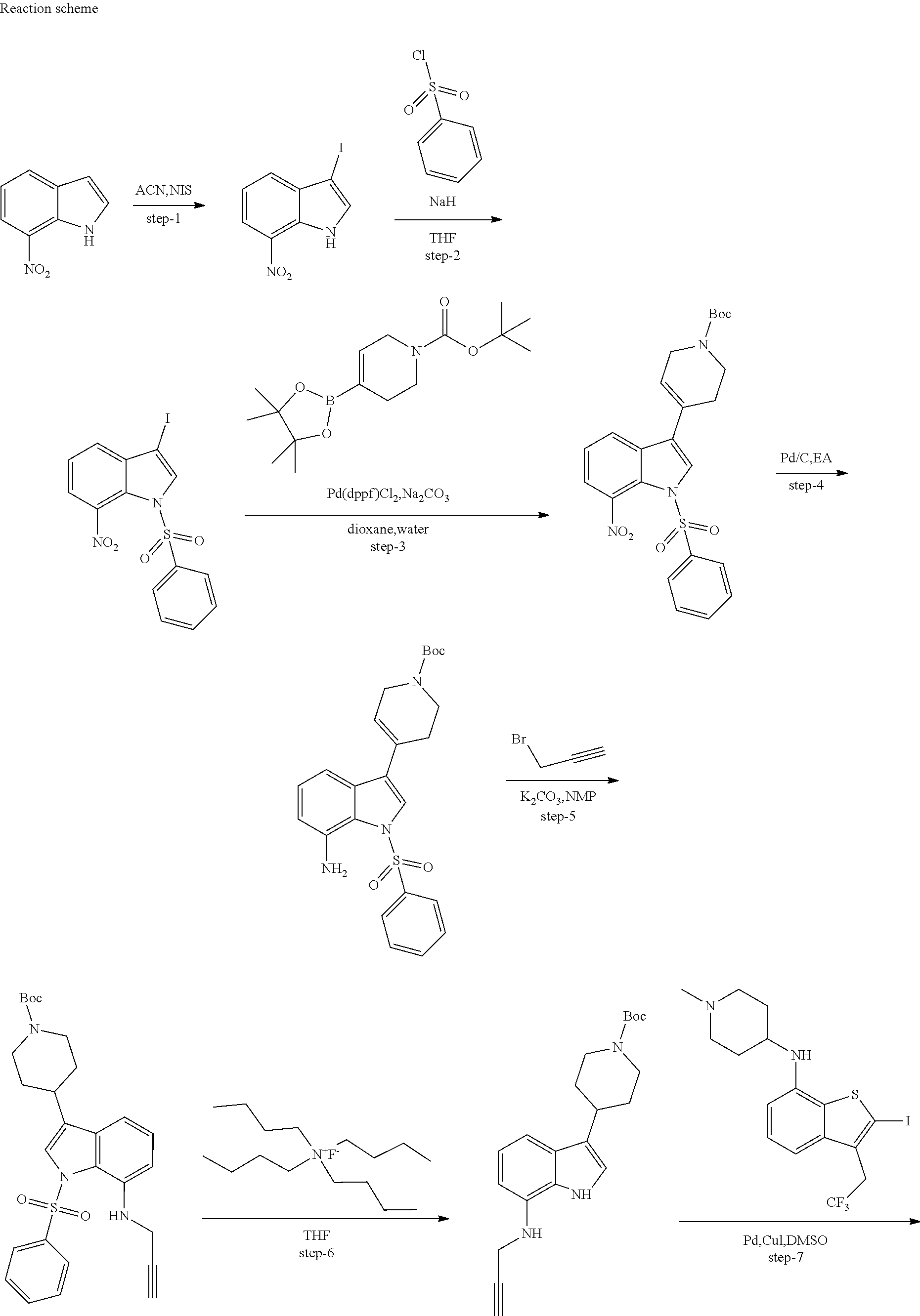

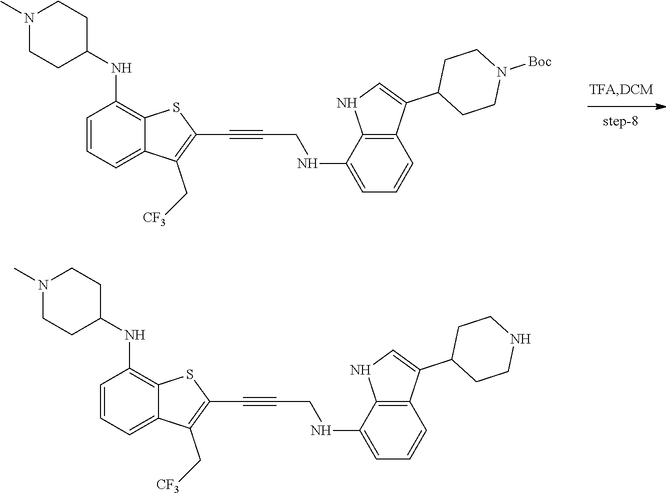 [SMILES: CN1CCC(Nc2cccc3c(CC(F)(F)F)c(C#CCNc4cccc5c(C6CCN(C(=O)OC(C)(C)C)CC6)c[nH]c45)sc23)CC1 ; CN1CCC(Nc2cccc3c(CC(F)(F)F)c(C#CCNc4cccc5c(C6CCNCC6)c[nH]c45)sc23)CC1]

### Experimental Details

### Step 1 3-iodo-7-nitro-1H-indole

Into a 100 ml 3-necked flask was placed 7-nitro-1H-indole (2.09 g, 12.88 mmol), NIS (3.49 g, 15.51 mmol), ACN (30 mL). The reaction was stirred at 80° C. for 1 h. The reaction was quenched with Na₂CO₃ aq (20 mL). The resulted solution was extracted with EA (3×50 mL), washed with brine (50 mL), dried over anhydrous sodium sulfate, filtered and concentrated under vacuum. This resulted in 2.86 g (9.92 mmol, 77.03% yield) of 3-iodo-7-nitro-1H-indole as light yellow solid. LCMS: m/z=289 [M+1]⁺.

### Step 2 3-iodo-7-nitro-1-(phenylsulfonyl)-1H-indole

Into a 50 ml 3-necked flask was placed 3-iodo-7-nitro-1H-indole (2.608 g, 9.05 mmol), THF (30 mL). The reaction was cooled to 0° C. Then NaH (0.522 g, mmol) was added with vigorous stirring at 0˜5° C. The reaction was stirred for about 1 h at 0˜5° C. Then benzenesulfonyl chloride (2.071 g, 11.72 mmol) was added. The reaction was stirred for about 1 h at room temperature. The reaction was quenched with water (20 mL). The resulted solution was extracted with EA (3×50 mL), washed with brine (50 mL), dried over anhydrous sodium sulfate, filtered and concentrated under vacuum. This resulted in 2.836 g (6.62 mmol, 73.14% yield) of 3-iodo-7-nitro-1-(phenylsulfonyl)-1H-indole as light yellow solid. LCMS: m/z=429 [M+1]⁺.

### Step 3 tert-butyl 4-(7-nitro-1-(phenylsulfonyl)-1H-indol-3-yl)-3,6-dihydropyridine-1(2H)-carboxylate

Into a 100 ml 3-necked flask was placed 3-iodo-7-nitro-1-(phenylsulfonyl)-1H-indole (2.401 g, 5.60 mmol), tert-butyl 4-(4,4,5,5-tetramethyl-1,3,2-dioxaborolan-2-yl)-3,6-dihydropyridine-1(2H)-carboxylate (3.089 g, 9.90 mmol), Pd(dppf)Cl₂ (0.836 g, 1.14 mmol), Na₂CO₃ (1.808 g, 17.05 mmol), water (4 mL), 1,4-Dioxane (20 mL). The reaction mixture was stirred under nitrogen at 50° C. for 5 h. The reaction was quenched with water (50 mL). The resulted solution was extracted with EA (3×100 mL), washed with brine (100 mL), dried over anhydrous sodium sulfate, filtered and concentrated under vacuum. The crude product was purified with silica gel column eluted with EA/hexane (v/v=3/7). This resulted in 1.585 g (3.27 μmol, 58.45% yield) of tert-butyl 4-(7-nitro-1-(phenylsulfonyl)-1H-indol-3-yl)-3,6-dihydropyridine-1(2H)-carboxylate as yell ow solid. LCMS: m/z=484 [M+1]⁺.

### Step 4 tert-butyl 4-(7-amino-1-(phenylsulfonyl)-1H-indol-3-yl) piperidine-1-carboxylate

Into a 100 ml 3-necked flask was placed tert-butyl 4-(7-nitro-1-(phenylsulfonyl)-1H-indol-3-yl)-3,6-dihydropyridine-1(2H)-carboxylate (0.901 g, 1.86 mmol), EA (8 mL), MeOH (2 mL), Pd/C (0.738 g, 3.46 mmol). The reaction mixture was stirred at RT for 2 h under hydrogen atmosphere. The reaction mixture was filtered through celite pad and the filter cake was washed with methanol (3×20 mL). The filtrate was concentrated under reduced pressure to afford the desired product. This resulted in 0.789 g (1.73 mmol 92.94% yield) of tert-butyl 4-(7-amino-1-(phenylsulfonyl)-1H-indol-3-yl)piperidine-1-carboxylate as faint yellow solid. LCMS: m/z=456 [M+1]⁺.

### Step 5 tert-butyl 4-(1-(phenylsulfonyl)-7-(prop-2-yn-1-ylamino)-1H-indol-3-yl)piperidine-1-carboxylate

Into a 25 mL 3-necked flask was placed tert-butyl 4-(7-amino-1-(phenylsulfonyl)-1H-indol-3-yl)piperidine-1-carboxylate (0.295 g, 647.54 μmol), 3-bromoprop-1-yne (0.082 g, 689.30 μmol), K₂CO₃ (0.266 g, 1.92 mmol), KI (0.219 g, 1.31 mmol), NMP (5 mL). The reaction mixture was stirred under nitrogen at 80° C. for 16 h. The reaction was quenched with water (10 mL). The resulted solution was extracted with EA (3×20 mL), washed with brine (20 mL), dried over anhydrous sodium sulfate, filtered and concentrated under vacuum. The crude product was purified by silica gel column eluted with EA/hexane (v/v=1/3). This resulted in 0.132 g (267.41 μmol, 41.29% yield) of tert-butyl 4-(1-(phenylsulfonyl)-7-(prop-2-yn-1-ylamino)-1H-indol-3-yl)piperidine-1-carboxylate as yellow solid. LCMS: m/z=494 [M+1]⁺.

### Step 6 tert-butyl 4-(7-(prop-2-yn-1-ylamino)-1H-indol-3-yl)piperidine-1-carboxylate

Into a 25 mL 3-necked flask was placed methyl tert-butyl 4-(1-(phenylsulfonyl)-7-(prop-2-yn-1-ylamino)-1H-indol-3-yl)piperidine-1-carboxylate (0.136 g, 275.51 mmol), tetrabutylammonium fluoride (72.03 g, 275.51 m mol), THF (2 mL). The reaction was stirred at 80° C. for 0.5 h. The reaction was quenched with NH₄Cl aq (10 mL). The resulted solution was extracted with EA (3×20 mL), washed with brine (20 mL), dried over anhydrous sodium sulfate, filtered and concentrated under vacuum. This resulted in 0.096 g (271.60 μmol, 98.57% yield) of tert-butyl 4-(7-(prop-2-yn-1-ylamino)-1H-indol-3-yl)piperidine-1-carboxylate as yellow oil. LCMS: m/z=354 [M+1]⁺.

### Step 7 tert-butyl 4-(7-((3-(7-((1-methylpiperidin-4-yl)amino)-3-(2,2,2-trifluoroethyl)benzo[b]thiophen-2-yl)prop-2-yn-1-yl)amino)-1H-indol-3-yl)piperidine-1-carboxylate

Into a 25 mL 3-necked flask was placed tert-butyl 4-(7-(prop-2-yn-1-ylamino)-1H-indol-3-yl)piperidine-1-carboxylate (0.092 g, 260.28 μmol), N-(2-iodo-3-(2,2,2-trifluoroethyl)benzo[b]thiophen-7-yl)-1-methylpiperidin-4-amine (0.079 g, 173.89 μmol), Pd(PPh₃)₂Cl₂ (0.038 g, 64.43 μmol), CuI (0.024 g, 128.41 μmol), DIEA (0.115 g, 892.05 μmol), methyl sulfoxide (2 mL). The reaction was stirred under nitrogen atmosphere at RT for 16 h. The reaction was quenched with water (5 mL). The resulted solution was extracted with EA (3×10 mL), washed with brine (10 mL), dried over anhydrous sodium sulfate, filtered and concentrated under vacuum. The crude was purified by silica gel column eluted with MeOH/DCM (v/v=1/9). This resulted in 0.073 g (107.37 μmol, 41.25% yield) of tert-butyl 4-(7-((3-(7-((1-methylpiperidin-4-yl)amino)-3-(2,2,2-trifluoroethyl)benzo[b]thiophen-2-yl)prop-2-yn-1-yl)amino)-1H-indol-3-yl)piperidine-1-carboxylate as brown oil. LCMS: m/z=680 [M+1]⁺.

### Step 8 N-(3-(7-((1-methylpiperidin-4-yl)amino)-3-(2,2,2-trifluoroethyl)benzo[b]thiophen-2-yl)prop-2-yn-1-yl)-3-(piperidin-4-yl)-1H-indol-7-amine (30)

Into a 8 mL sealed tube was placed tert-butyl 4-(7-((3-(7-((1-methylpiperidin-4-yl)amino)-3-(2,2,2-trifluoroethyl)benzo[b]thiophen-2-yl)prop-2-yn-1-yl)amino)-1H-indol-3-yl)piperidine-1-carboxylate (0.073 g, 107.37 μmol), TFA (1 mL), DCM (1 mL). The reaction was stirred at RT for 1 h. The mixture was purified by preparative HPLC (Mobile Phase A: water (0.1% ammonium hydroxide), Mobile Phase B: ACN; Flow rate: 40 m L/min; Gradient: 30-70-100-100% B (2-30-60-90 min); 228 nm; RT: 36.80-39.49) to provide the desired pro duct. This resulted in 0.009 g (15.52 μmol, 14.45% yield) of N-(3-(7-((1-methylpiperidin-4-yl)amino)-3-(2,2,2-trifluoroethyl)benzo[b]thiophen-2-yl)prop-2-yn-1-yl)-3-(piperidin-4-yl)-1H-indol-7-amine (30) as white solid. LCMS: m/z=580 [M+1]⁺.

¹H NMR (400 MHz, DMSO-d₆) δ 10.34 (s, 1H), 7.37-7.21 (m, 2H), 7.14 (d, J=8.0 Hz, 1H), 7.01 (s, 1H), 6.95 (d, J=8.1 Hz, 1H), 6.84 (t, J=7.7 Hz, 1H), 6.66 (d, J=7.8 Hz, 1H), 6.47 (d, J=7.4 Hz, 1H), 5.80 (t, J=6.1 Hz, 1H), 5.29 (d, J=8.0 Hz, 1H), 4.42 (d, J=6.0 Hz, 2H), 3.82-3.76 (m, 2H), 3.02 (d, J=12.0 Hz, 2H), 2.75 (d, J=10.5 Hz, 2H), 2.69-2.60 (m, 3H), 2.17 (s, 3H), 1.99 (t, J=11.5 Hz, 2H), 1.86 (d, J=11.2 Hz, 4H), 1.54 (d, J=11.2 Hz, 4H), 1.24 (s, 1H).

### Example 31

### N-(3-(7-((1-methylpiperidin-4-yl)amino)-3-(2,2,2-trifluoroethyl)benzo[b]thiophen-2-yl)prop-2-yn-1-yl)-4-(methylsulfonyl)-1H-indol-7-amine (31)

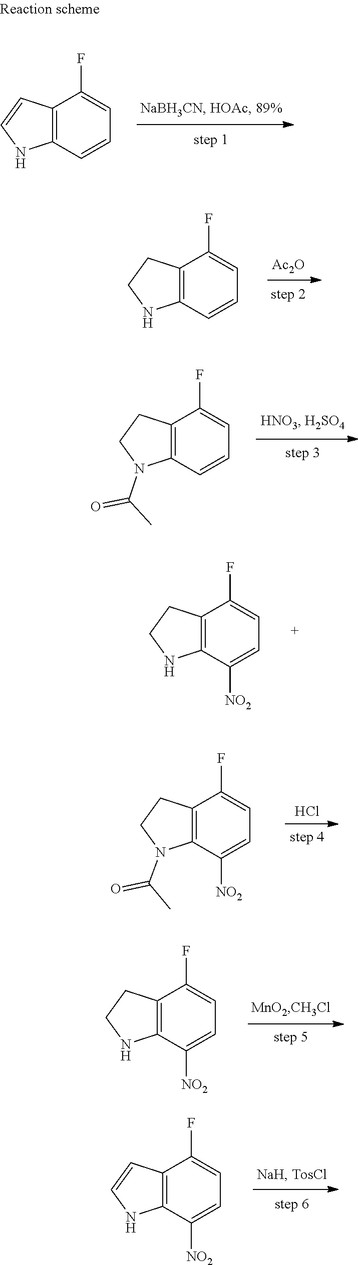

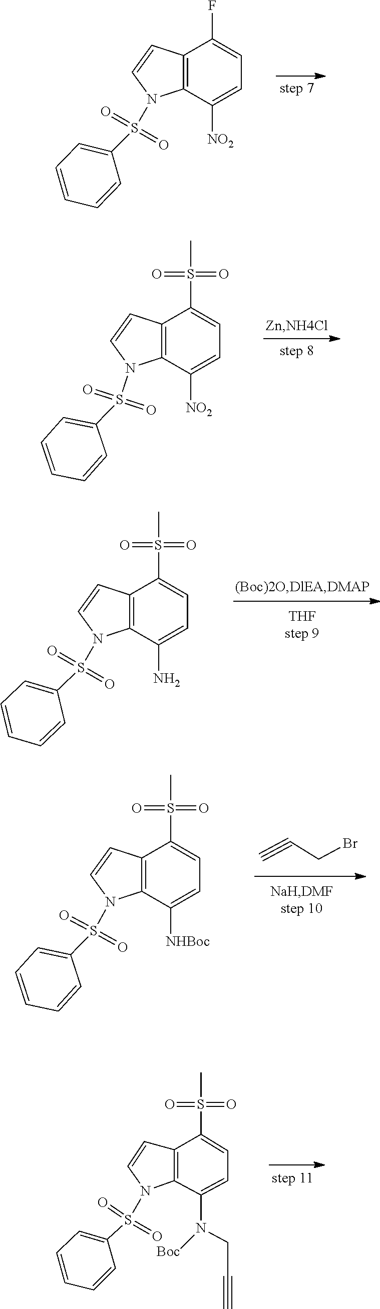

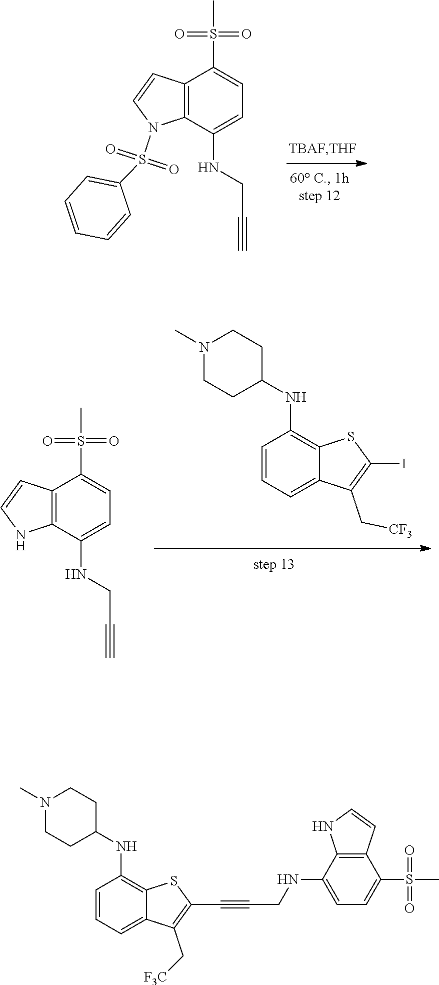

### Experimental Details

### Step 1. 4-fluoroindoline

Into a 250 mL flask was placed 4-fluoro-1H-indole (15.12 g, 111.88 mmol), acetic acid (80 mL). The reaction was stirred at RT while sodium cyanoboronhydride (13.99 g, 326.29 mmol) was added in batches. The reaction was stirred at RT for 2 h. The reaction was quenched with H₂O (200 mL), extracted with EA (3×100 mL), concentrated under vacuum. The crude was purified by silica gel column eluted with EA/hexane (v/v=1/1) to afford 4-fluoroindoline (14.35 g, 93.51%) as yellow solid. LCMS: m/z=138 [M+1]⁺.

### Step 2. 1-(4-fluoroindolin-1-yl)ethan-1-one

Into a 250 mL flask was placed acetic anhydride (100 mL). The reaction was stirred at 0° C. and 4-fluoroindoline (14.41 g, 105.06 mmol) was added in batches. The reaction was stirred at RT for 2 h. The reaction was quenched with H₂O (200 mL), extracted with EA (3×100 mL), concentrated under vacuum. The c rude was purified by silica gel column eluted with EA/Hexane (v/v=1/3) to afford 1-(4-fluoroindolin-1-yl) ethan-1-one (12.16 g, 64.58%) as yellow solid. LCMS: m/z=180 [M+1]⁺.

### Step 3. 1-(4-fluoro-7-nitroindolin-1-yl)ethan-1-one

Into a 250 mL flask was placed 1-(4-fluoroindolin-1-yl)ethan-1-one (6.07 g, 33.87 mmol), sulfuric acid (50 mL). The reaction mixture was stirred at −10° C. under N₂ atmosphere and nitric acid (3.0 g, 47.60 mmol) was added dropwise. The reaction was stirred at rt for 1 h. The reaction was quenched with H₂O (100 m L), extracted with EA (3×100 mL), concentrated under vacuum. The crude was purified by silica gel column eluted with EA/Hexane (v/v=1/3) to afford 1-(4-fluoro-7-nitroindolin-1-yl)ethan-1-one (1.67 g, 21.99%) as yellow solid. LCMS: m/z=225 [M+1]⁺.

### Step 4. 4-fluoro-7-nitroindoline

Into a 250 mL flask was placed 1-(4-fluoro-7-nitroindolin-1-yl)ethan-1-one (2.146 g, 9.57 mmol), hydrogen chloride (50 mL). The reaction mixture was stirred at 100° C. for 1 h. The reaction was cooled to rt, concentrated under vacuum. The reaction was quenched with aq·Na₂CO₃ (50 mL), extracted with EA (3×100 m L), concentrated under vacuum. The crude was purified by silica gel column eluted with EA/Hexane (v/v=1/1) to afford 4-fluoro-7-nitroindoline (1.69 g, 96.98%) as yellow solid. LCMS: m/z=183 [M+1]⁺.

### Step 5. 4-fluoro-7-nitro-1H-indole

Into a 250 mL flask was placed 4-fluoro-7-nitroindoline (1.351 g, 7.41 mmol), manganese oxide (6.319 g, 72.68 mmol), chloroform (50 mL). The reaction was stirred at 80° C. for 16 h before cooled to rt. The reaction mixture was filtrated, washed with EA (50 mL), concentrated under vacuum. The crude was purified by silica gel column eluted with EA/Hexane (v/v=1/3) to afford 4-fluoro-7-nitro-1H-indole (1.181 g, 88.39%) as yellow solid. LCMS: m/z=181 [M+1]⁺.

### Step 6. 4-fluoro-7-nitro-1-(phenylsulfonyl)-1H-indole

Into a 100 mL flask was placed 4-fluoro-7-nitro-1H-indole (1.184 g, 6.57 mmol), DMF (30 mL). The reaction mixture was stirred at 0° C. under N₂ atmosphere and NaH (0.459 g, 19.12 mmol) was added portion wise. The reaction was stirred at rt for 30 min and then benzenesulfonyl chloride (2.541 g, 14.38 mmol) was added dropwise. The reaction was stirred at RT for 2 h. The reaction was quenched with H₂O (50 mL), extracted with EA (3×100 mL), concentrated under vacuum. The crude was purified by silica gel column eluted with EA/Hexane (v/v=1/3) to afford 4-fluoro-7-nitro-1-(phenylsulfonyl)-1H-indole (2.036 g, 96.71%) as yellow solid. LCMS: m/z=321 [M+1]⁺.

### Step 7. 4-(methylsulfonyl)-7-nitro-1-(phenylsulfonyl)-1H-indole

Into a 100 mL flask was placed 4-fluoro-7-nitro-1-(phenylsulfonyl)-1H-indole (2.01 g, 6.27 mmol), sodium methanesulfinate (1.366 g, 13.38 mmol), DMF (30 mL). The reaction was stirred at 80° C. for 12 h. The reaction was cooled to rt and quenched with H₂O (100 mL), extracted with EA (3×100 mL), concentrated under vacuum. The crude was purified by silica gel column eluted with EA/Hexane (v/v=1/2) to afford 4-(methylsulfonyl)-7-nitro-1-(phenylsulfonyl)-1H-indol (1.24 g, 51.94%) as yellow solid. LCMS: m/z=381 [M+1]⁺.

### Step 8. 4-(methylsulfonyl)-1-(phenylsulfonyl)-1H-indol-7-amine

Into a 100 mL flask was placed 4-(methylsulfonyl)-7-nitro-1-(phenylsulfonyl)-1H-indole (1.19 g, 3.12 mmol), zinc (1.499 g, 22.92 mmol), NH₄Cl (1.367 g, 25.55 mmol), ethanol (50 mL), water (10 mL). The reaction was stirred at 70° C. for 3 h. The reaction mixture was filtrated, washed with EA (50 mL), concentrated under vacuum. The crude was purified by silica gel column eluted with EA/hexane (v/v=1/3) to afford 4-(methylsulfonyl)-1-(phenylsulfonyl)-1H-indol-7-amine (0.942 g, 85.93%) as yellow solid. LCMS: m/z=351 [M+1]⁺.

### Step 9. tert-butyl (4-(methylsulfonyl)-1-(phenylsulfonyl)-1H-indol-7-yl)carbamate

Into a 100 mL flask was placed 4-(methylsulfonyl)-1-(phenylsulfonyl)-1H-indol-7-amine (0.616 g, 1.75 mmol), di-tert-butyl dicarbonate (0.443 g, 2.02 mmol), TEA (0.659 g, 6.51 mmol), THF (10 mL), N-(4-pyridyl) dimethylamine (0.041 g, 335.60 μmol), The reaction mixture was stirred at rt for 2 h before quenched with H₂O (30 mL), extracted with EA (3×50 mL), concentrated under vacuum. The crude was purified by silica gel column eluted with EA/Hexane (v/v=1/2) to afford tert-butyl N-[1-(benzenesulfonyl)-4-methylsulfonyl-indol-7-yl]carbamate (0.738 g, 93.18%) as yellow solid. LCMS: m/z=451 [M+1]⁺.

### Step 10. tert-butyl (4-(methylsulfonyl)-1-(phenylsulfonyl)-1H-indol-7-yl)(prop-2-yn-1-yl)carbamate

Into a 8 mL reaction vial was placed tert-butyl (4-(methylsulfonyl)-1-(phenylsulfonyl)-1H-indol-7-yl)carbamate (0.742 g, 1.64 mmol), DMF (10 mL). The reaction mixture was stirred at 0° C. before NaH (0.221 g, 9.20 mmol) was added in portion-wise. The reaction was stirred at rt for 30 min. 3-bromoprop-1-yne (0.888 g, 7.46 mmol) was added to above mixture. The reaction was stirred at rt for 12 h before quenched with H₂O (30 mL), extracted with EA (3×50 mL), concentrated under vacuum. The crude was purified by silica gel column eluted with EA/Hexane (v/v=1/2) to afford tert-butyl (4-(methylsulfonyl)-1-(phenylsulfonyl)-1H-indol-7-yl)(prop-2-yn-1-yl)carbamate (0.533 g, 66.23%) as yellow solid. LCMS: m/z=489 [M+1]⁺.

### Step 11. 4-(methylsulfonyl)-1-(phenylsulfonyl)-N-(prop-2-yn-1-yl)-1H-indol-7-amine

Into a 50 mL flask was placed tert-butyl (4-(methylsulfonyl)-1-(phenylsulfonyl)-1H-indol-7-yl)(prop-2-yn-1-yl) carbamate (0.456 g, 933.32 μmol), DCM (6 mL), trifluoroacetic acid (2 mL). The reaction was stirred at RT for 1 h. The reaction was quenched with aq·Na₂CO₃ (20 mL), extracted with DCM (3×30 mL), concentrated under vacuum. The crude was purified by prep-TLC with DCM/MeOH (v/v=20/1) to afford 4-(m ethylsulfonyl)-1-(phenylsulfonyl)-N-(prop-2-yn-1-yl)-1H-indol-7-amine (0.050 g, 13.79%) as yellow solid. LCMS: m/z=389 [M+1]⁺.

### Step 12. 4-(methylsulfonyl)-1-(phenylsulfonyl)-N-(prop-2-yn-1-yl)-1H-indol-7-amine

Into a 8 mL reaction vial was placed 4-(methylsulfonyl)-1-(phenylsulfonyl)-N-(prop-2-yn-1-yl)-1H-indol-7-am me (0.049 g, 126.13 μmol), TBAF (1 M in THF) (2 mL), THF (0.5 mL). The reaction was stirred at 80° C. for 1 h. The reaction was quenched with H₂O (10 mL), extracted with EA (3×30 mL), concentrated under vacuum. The crude was purified by prep-TLC with DCM/MeOH (v/v=20/1) to afford 4-(methylsulfonyl)-N-(prop-2-yn-1-yl)-1H-indol-7-amine (0.023 g, 73.43%) as yellow solid. LCMS: m/z=249 [M+1]⁺.

### Step 13. N-(3-(7-((1-methylpiperidin-4-yl)amino)-3-(2,2,2-trifluoroethyl)benzo[b]thiophen-2-yl)prop-2-yn-1-yl)-4-(methylsulfonyl)-1H-indol-7-amine (31)

Into a 8 mL reaction vial was placed N-(2-iodo-3-(2,2,2-trifluoroethyl)benzo[b]thiophen-7-yl)-1-methylpiperidin-4-amine (0.050 g, 110.06 μmol), 4-(methylsulfonyl)-N-(prop-2-yn-1-yl)-1H-indol-7-amine (0.021 g, 84.57 μmol), bis(triphenylphosphine)palladium(II) chloride (0.018 g, 25.49 μmol), N,N-Diisopropylethylamine (0.031 g, 239.85 μmol), CuI (0.015 g, 78.76 μmol), methyl sulfoxide (2 mL). The reaction was stirred at rt for 1 h under N₂ atmosphere. The reaction was quenched with H₂O (10 mL), extracted with EA (3×20 mL), concentrated under vacuum. The mixture was purified by preparative HPLC (Mobile Phase A: water (10 mmoL/L ammonium hydroxide), Mobile Phase B: ACN; Flow rate: 40 mL/min; Gradient: 25-55-80% B (2-30-60 min); 228 nm; RT: 36.803-39.498 min) to afford N-(3-(7-((1-methylpiperidin-4-yl)amino)-3-(2,2,2-trifluoro ethyl)benzo[b]thiophen-2-yl)prop-2-yn-1-yl)-4-(methylsulfonyl)-1H-indol-7-amine (31) (0.007 g, 11.06%) as of f-white solid. LCMS: m/z=575 [M+1]⁺.

¹H NMR (400 MHz, DMSO) δ 11.30 (s, 1H), 7.52 (d, J=3.0 Hz, 1H), 7.46 (d, J=8.2 Hz, 1H), 7.25 (t, J=7.8 Hz, 1H), 7.15 (d, J=7.9 Hz, 1H), 6.88 (s, 1H), 6.67 (t, J=5.8 Hz, 2H), 6.61 (d, J=8.3 Hz, 1H), 5.31 (d, J=8.0 Hz, 1H), 4.56 (d, J=3.8 Hz, 2H), 3.84 (q, J=11.1 Hz, 2H), 3.05 (s, 3H), 2.75 (d, J=11.4 Hz, 2H), 2.53 (s, 1H), 2.18 (d, J=13.8 Hz, 3H), 1.98 (t, J=10.9 Hz, 2H), 1.88 (d, J=11.4 Hz, 2H), 1.54 (dd, J=21.3, 10.4 Hz, 2H).

### Example 32

### N-(2-(3-((2-methoxy-4-(methylsulfonyl)phenyl)amino)prop-1-yn-1-yl)-3-(2,2,2-trifluoroethyl)benzo[b]thiophen-7-yl)-3-methyl-3-azabicyclo[3.2.0]heptan-6-amine (32)

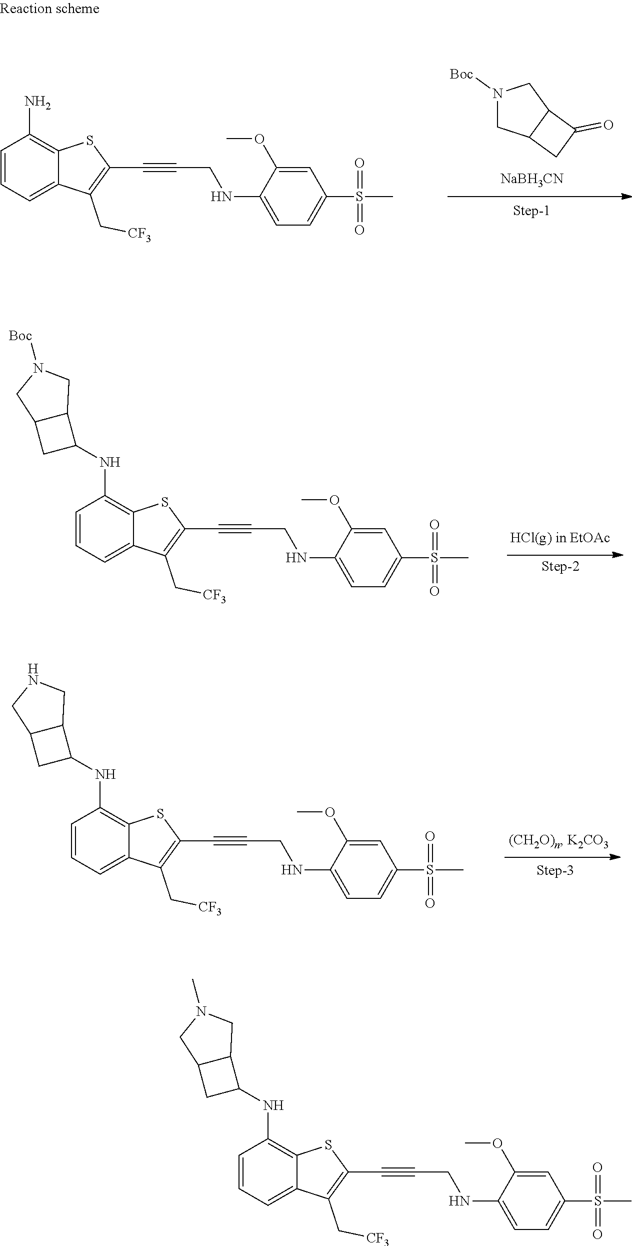 [SMILES: COc1cc(S(C)(=O)=O)ccc1NCC#Cc1sc2c(N)cccc2c1CC(F)(F)F ; CC(C)(C)OC(=O)N1CC2CC(=O)C2C1 ; "NaBH3CN" ; COc1cc(S(C)(=O)=O)ccc1NCC#Cc1sc2c(NC3CC4CN(C(=O)OC(C)(C)C)CC43)cccc2c1CC(F)(F)F ; COc1cc(S(C)(=O)=O)ccc1NCC#Cc1sc2c(NC3CC4CNCC43)cccc2c1CC(F)(F)F ; COc1cc(S(C)(=O)=O)ccc1NCC#Cc1sc2c(NC3CC4CN(C)CC43)cccc2c1CC(F)(F)F]

### Experimental Details

### Step 1. tert-butyl 6-((2-(3-((2-methoxy-4-(methylsulfonyl)phenyl)amino)prop-1-yn-1-yl)-3-(2,2,2-trifluoroethyl)benzo[b]thiophen-7-yl)amino)-3-azabicyclo[3.2.0]heptane-3-carboxylate

Into a 4 mL vail was placed 2-(3-((2-methoxy-4-(methylsulfonyl)phenyl)amino)prop-1-yn-1-yl)-3-(2,2,2-trifluoroethyl)benzo[b]thiophen-7-amine (0.099 g, 211.31 μmol), tert-butyl 6-oxo-3-azabicyclo[3.2.0]heptane-3-carboxylate (0.250 g, 1.18 mmol). The opened-vail was heated to 150° C. and stirred for 0.5 h. The reaction was cooled to room temperature, and sodium cyanoborohydride (211 mg, 3.43 mmol), acetic acid (0.01 μmol), Ethanol (0.5 mL) was added. The reaction was stirred for 1 h at room temperature. LCMS showed the reaction was complete, the reaction was concentrated under vacuum and purified with C₁₈ column, eluted with ACN/water (v/v=1/3) to afford tert-butyl 6-((2-(3-((2-methoxy-4-(methylsulfonyl)phenyl)amino)prop-1-yn-1-yl)-3-(2,2,2-trifluoroethyl)benzo[b]thiophen-7-yl)amino)-3-azabicyclo[3.2.0]heptane-3-carboxylate (0.288 g, 433.89 μmol, 205.33% yield) as clear oil. LCMS: m/z=664[M+1]⁺.

### Step 2. N-(2-(3-((2-methoxy-4-(methylsulfonyl)phenyl)amino)prop-1-yn-1-yl)-3-(2,2,2-trifluoroethyl)benzo[b]thiophen-7-yl)-3-azabicyclo[3.2.0]heptan-6-amine

Into a 4 mL flask was placed tert-butyl 6-((2-(3-((2-methoxy-4-(methylsulfonyl)phenyl)amino)prop-1-yn-1-yl)-3-(2,2,2-trifluoroethyl)benzo[b]thiophen-7-yl)amino)-3-azabicyclo[3.2.0]heptane-3-carboxylate (0.208 g, 313.36 μmol), HCl (g) in EtOAc (1M, 0.5 mL) and stirred for 1 h at room temperature. LCMS showed the reaction was complete, the reaction was quenched with sat·NaHCO₃ aq. to pH 8˜9 under 0° C., extracted with EA (3 mL×3). The combined organic layers were washed with water (3 mL) and brine (3 mL) successively, separated, then concentrated with vacuum. The residue was purified with C₁₈ column, eluted with ACN/water (v/v=1/2) to afford N-(2-(3-((2-methoxy-4-(methylsulfonyl)phenyl)amino)prop-1-yn-1-yl)-3-(2,2,2-trifluoroethyl)benzo[b]thiophen-7-yl)-3-azabicyclo[3.2.0]heptan-6-amine.

LCMS: m/z=564[M+1]⁺.

### Step 3. N-(2-(3-((2-methoxy-4-(methylsulfonyl)phenyl)amino)prop-1-yn-1-yl)-3-(2,2,2-trifluoroethyl)benzo[b]thiophen-7-yl)-3-methyl-3-azabicyclo[3.2.0]heptan-6-amine (32)

Into a 4 mL flask was placed N-(2-(3-((2-methoxy-4-(methylsulfonyl)phenyl)amino)prop-1-yn-1-yl)-3-(2,2,2-tri fluoroethyl)benzo[b]thiophen-7-yl)-3-azabicyclo[3.2.0]heptan-6-amine (0.047 g, 70.81 μmol), paraformaldehyde (0.004 g, 133.22 μmol), sodium cyanoboronhydride (37 mg, 606.40 μmol), EtOH (0.5 mL), glacial acetic acid (0.01 mL). The reaction was stirred overnight at room temperature. LCMS showed the reaction was complete. The reaction was quenched with water (4 mL), extracted with EA (2 mL×2). The combined organic layers were washed with water (2 mL) and brine (2 mL) successively, separated, then concentrated with vacuum. The residue was purified with prep-HPLC (Mobile Phase A: water (ammonium hydroxide), Mobile Phase B: ACN; Flow rate: 70 mL/min; Gradient: 50-85-100% B (2-30-60 min); 270 nm; RT: 38.379-39.803 min) to afford N-(2-(3-((2-methoxy-4-(methylsulfonyl)phenyl)amino)prop-1-yn-1-yl)-3-(2,2,2-trifluoroethyl)benzo[b]thiophen-7-yl)-3-methyl-3-azabicyclo[3.2.0]heptan-6-amine (32) (5 mg, 8.66 μmol, 12.22% yield) as off-white solid. LCMS: m/z=578[M+1]⁺.

¹H NMR (400 MHz, DMSO-d₆) δ 7.41-7.38 (m, 1H), 7.28-7.23 (m, 2H), 7.19-7.15 (m, 1H), 6.90 (d, J=8.2 Hz, 1H), 6.53-6.47 (m, 2H), 5.33 (d, J=7.4 Hz, 1H), 4.39 (d, J=6.2 Hz, 2H), 4.12-4.08 (m, 1H), 3.90 (s, 3H), 3.84-3.79 (m, 2H), 3.10 (s, 3H), 2.72 (d, J=9.1 Hz, 1H), 2.64-2.58 (m, 2H), 2.27 (s, 3H), 1.99-1.94 (m, 2H), 1.86-1.81 (m, 2H).

### Example 33

### N-(2-(3-((2-methoxy-4-(methylsulfonyl)phenyl)amino)prop-1-yn-1-yl)-3-(2,2,2-trifluoroethyl)benzo[b]thiophen-7-yl)-7-methyl-7-azaspiro[3.5]nonan-2-amine (33)

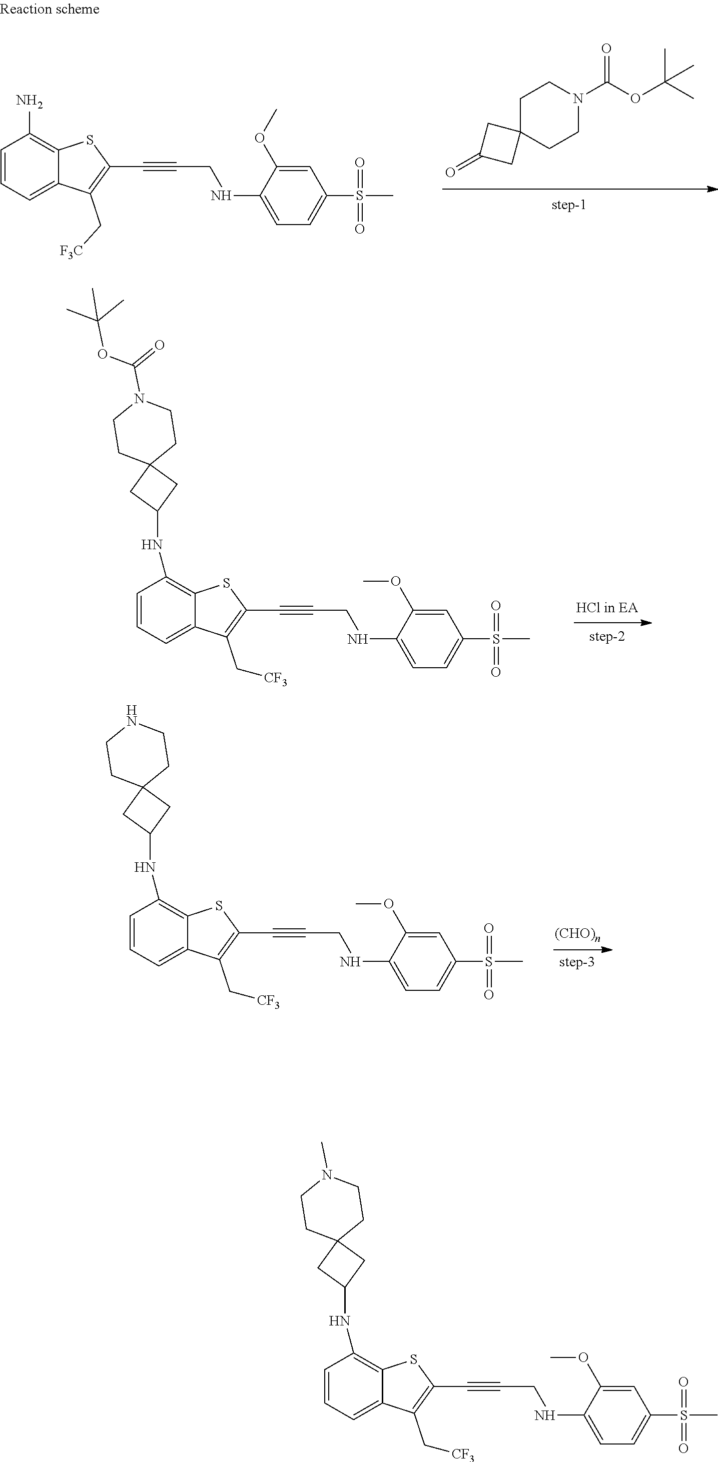

### Experimental Details

### Step 1. tert-butyl 2-((2-(3-((2-methoxy-4-(methylsulfonyl)phenyl)amino)prop-1-yn-1-yl)-3-(2,2,2-trifluoroethyl)benzo[b]thiophen-7-yl)amino)-7-azaspiro[3.5]nonane-7-carboxylate

Into a 8 mL flask was placed titanium ethoxide (0.072 g, 315.64 μmol), 2-(3-((2-methoxy-4-(methylsulfonyl) phenyl)amino)prop-1-yn-1-yl)-3-(2,2,2-trifluoroethyl)benzo[b]thiophen-7-amine (0.061 g, 130.20 μmol), tert-butyl 2-oxo-7-azaspiro[3.5]nonane-7-carboxylate (0.164 g, 685.30 μmol), toluene (2 mL). The reaction mixture was stirred at 110° C. for 2 h. The reaction was concentrated under vacuum and dissolved with methanol (2 mL). Then sodium cyanoboronhydride (0.034 g, 792.99 μmol) was added. The reaction mixture was stirred at room temperature for another 15 h. The reaction was quenched by the addition of water (10 mL) and extracted with EA (2×10 mL). The organic layers was combined, washed with brine (10 mL), dried over anhydrous Na₂SO₄ and concentrated under vacuum. The residue was applied onto a silica gel column eluted with EA/heptane (v/v=1/3). This resulted in 0.080 g (88.82% yield) of tert-butyl 2-((2-(3-((2-methoxy-4-(methylsulfonyl)phenyl)amino)prop-1-yn-1-yl)-3-(2,2,2-trifluoroethyl)benzo[b]thiophen-7-yl)amino)-7-azaspiro[3.5]nonane-7-carboxylate as yellow oil. LCMS: m/z=692 [M+1]⁺.

### Step 2. N-(2-(3-((2-methoxy-4-(methylsulfonyl)phenyl)amino)prop-1-yn-1-yl)-3-(2,2,2-trifluoroethyl)benzo[b]thiophen-7-yl)-7-azaspiro[3.5]nonan-2-amine

Into a 8 mL flask was placed tert-butyl 2-((2-(3-((2-methoxy-4-(methylsulfonyl)phenyl)amino)prop-1-yn-1-yl)-3-(2,2,2-trifluoroethyl)benzo[b]thiophen-7-yl)amino)-7-azaspiro[3.5]nonane-7-carboxylate (0.079 g, 114.19 μmol), hydrogen chloride (4 M in EA, 2 mL). The reaction mixture was stirred at room temperature for 0.5 h. Then saturated sodium hydrogen carbonate aqueous solution was added to the mixture until pH=7-8. The resulting solution was extracted with EA (2×10 mL). The organic layers was combined, washed with brine (10 mL), dried over anhydrous Na₂SO₄ and concentrated under vacuum. This resulted in 0.067 g (99.16% yield) of N-(2-(3-((2-methoxy-4-(methylsulfonyl)phenyl)amino)prop-1-yn-1-yl)-3-(2,2,2-trifluoroethyl)benzo[b]thiophen-7-yl)-7-azaspiro[3.5]nonan-2-amine as yellow solid. LCMS: m/z=592[M+1]⁺.

### Step 3. N-(2-(3-((2-methoxy-4-(methylsulfonyl)phenyl)amino)prop-1-yn-1-yl)-3-(2,2,2-trifluoroethyl)benzo[b]thiophen-7-yl)-7-methyl-7-azaspiro[3.5]nonan-2-amine (33)

Into a 8 mL flask was placed N-(2-(3-((2-methoxy-4-(methylsulfonyl)phenyl)amino)prop-1-yn-1-yl)-3-(2,2,2-tri fluoroethyl)benzo[b]thiophen-7-yl)-7-azaspiro[3.5]nonan-2-amine (0.066 g, 111.54 μmol), paraformaldehyde (0.007 g, 233.13 μmol), methanol (4 mL), acetic acid (0.1 mL). The reaction mixture was stirred at room temperature for 19 h. Then sodium cyanoboronhydride (0.009 g, 209.91 μmol) was added. The reaction mixture was stirred at room temperature for another 4 h. The reaction was quenched by the addition of water (2 mL) and extracted with EA (2×5 mL). The organic layers was combined, washed with brine (10 mL) and concentrated under vacuum. The mixture was purified by preparative HPLC (Mobile Phase A: water (10 mmoL/L ammonium hydroxide), Mobile Phase B: MeOH; Flow rate: 40 mL/min; Gradient: 50-80-100% B (2-30-60 min); 278 nm; RT: 39.752-43.259). This resulted in 0.002 g (2.96% yield) of N-(2-(3-((2-methoxy-4-(methylsulfonyl)phenyl)amino)prop-1-yn-1-yl)-3-(2,2,2-trifluoroethyl)benzo[b]thiophen-7-yl)-7-methyl-7-azaspiro[3.5]nonan-2-amine (33) as white solid. LCMS: m/z=606 [M+1]⁺.

### Example 34

### 3-fluoro-N-(2-(3-((2-methoxy-4-(methylsulfonyl)phenyl)amino)prop-1-yn-1-yl)-3-(2,2,2-trifluoroethyl)benzol[b]thiophen-7-yl)-1-methylpiperidin-4-amine (34)

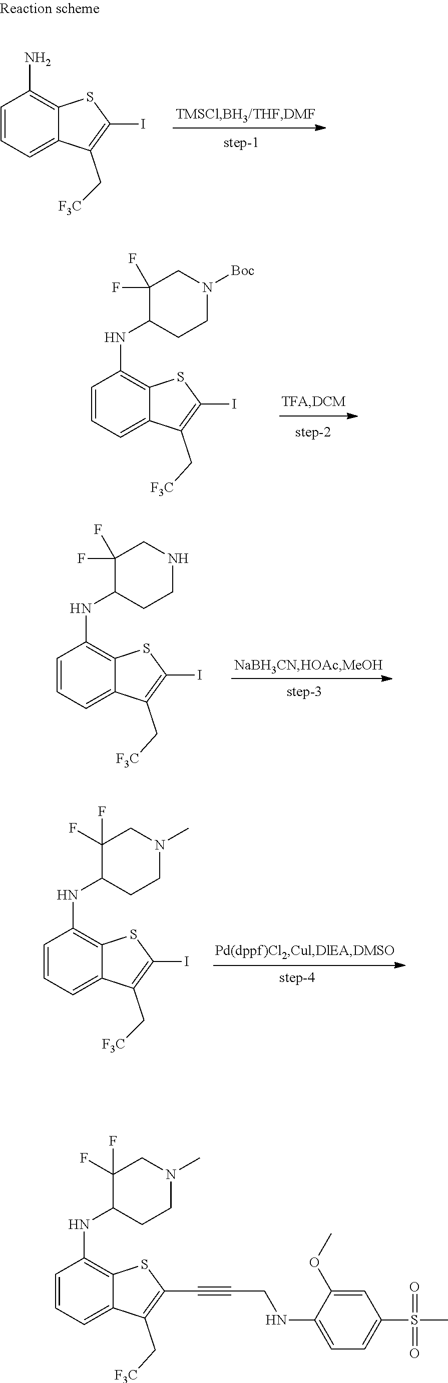 [SMILES: Nc1cccc2c(CC(F)(F)F)c(I)sc12 ; CC(C)(C)OC(=O)N1CCC(Nc2cccc3c(CC(F)(F)F)c(I)sc23)C(F)(F)C1 ; FC(F)(F)Cc1c(I)sc2c(NC3CCNCC3(F)F)cccc12 ; CN1CCC(Nc2cccc3c(CC(F)(F)F)c(I)sc23)C(F)(F)C1 ; COc1cc(S(C)(=O)=O)ccc1NCC#Cc1sc2c(NC3CCN(C)CC3(F)F)cccc2c1CC(F)(F)F]

### Experimental Details

### Step 1. tert-butyl 3,3-difluoro-4-((2-iodo-3-(2,2,2-trifluoroethyl)benzo[b]thiophen-7-yl)amino)piperidine-1-carboxylate

A mixture of 2-iodo-3-(2,2,2-trifluoroethyl)benzo[b]thiophen-7-amine (0.506 g, 1.42 mmol), tert-butyl 3,3-di fluoro-4-oxopiperidine-1-carboxylate (1.053 g, 4.48 mmol), and TMSCl (1.680 g, 15.46 mmol) in DMF (10 mL) was degassed and purged with N₂(g), and then BH₃·THF (1 M, 15 mL) was added. The mixture was stirred at 0° C. for 1 h. The reaction mixture was quenched with water (20 mL) at 0° C. and extracted with EA (60 mL×2). The combined organic layers was washed with brine (30 mL), dried over anhydrous s odium sulfate, filtered, and concentrated under reduced pressure. The residue was purified by silica gel column, eluted with EA/hexane (v/v=1/2) to afford tert-butyl 3,3-difluoro-4-((2-iodo-3-(2,2,2-trifluoroethyl)benzo[b]thiophen-7-yl)amino)piperidine-1-carboxylate (0.932 g, crude) as a little yellow oil. LCMS: m/z=577 [M+1]⁺

### Step 2. 3,3-difluoro-N-(2-iodo-3-(2,2,2-trifluoroethyl)benzo[b]thiophen-7-yl)piperidin-4-amine

Into a 100-mL round-bottom flask was placed tert-butyl 3,3-difluoro-4-((2-iodo-3-(2,2,2-trifluoroethyl)benzo[b]thiophen-7-yl)amino)piperidine-1-carboxylate (0.871 g, 1.51 mmol), DCM (10 mL) and TFA (2 mL). the reaction mixture was stirred at room temperature for 1 h. The reaction was adjusted to pH=9 with NaHCO₃ (aq.) and extracted with EA (100 mL×2). The combined organic layers was washed with brine (40 mL), separated and concentrated under vacuum. This resulted in 0.656 g (91.15%) of 3,3-difluoro-N-(2-iodo-3-(2,2,2-trifluoroethyl)benzo[b]thiophen-7-yl)piperidin-4-amine as yellow oil. LCMS: m/z=477 [M+1]⁺.

### Step 3. 3-fluoro-N-(2-iodo-3-(2,2,2-trifluoroethyl)benzo[b]thiophen-7-yl)-1-methylpiperidin-4-amine

To a solution of 3,3-difluoro-N-(2-iodo-3-(2,2,2-trifluoroethyl)benzo[b]thiophen-7-yl)piperidin-4-amine (0.628 g, 1.32 mmol), paraformaldehyde (0.082 g, 2.73 mmol) in 10 mL MeOH. The mixture was stirred at room temperature for 0.5 h. Then NaBH₃CN (0.289 g, 6.74 mmol) and HOAc (0.002 mL) were added. The reaction mixture was stirred at room temperature for 12 hours. The residue was purified by silica gel column, eluted with EA/hexane (v/v=1/2) to afford 3-fluoro-N-(2-iodo-3-(2,2,2-trifluoroethyl)benzo[b]thiophen-7-yl)-1-methylpiperidin-4-amine (0.544 g, 84.15% yield) as yellow oil. LCMS: m/z=491 [M+1]⁺

### Step 4. 3-fluoro-N-(2-(3-((2-methoxy-4-(methylsulfonyl)phenyl)amino)prop-1-yn-1-yl)-3-(2,2,2-trifluoroethyl) benzo[b]thiophen-7-yl)-1-methylpiperidin-4-amine (34)

Into a 8-mL sealed tube purged and maintained with an inert atmosphere of nitrogen, was placed 3-fluoro-N-(2-iodo-3-(2,2,2-trifluoroethyl)benzo[b]thiophen-7-yl)-1-methylpiperidin-4-amine (0.097 g, 0.20 mmol), 2-methoxy-4-(methylsulfonyl)-N-(prop-2-yn-1-yl)aniline (0.090 g, 0.38 mmol), Pd(dppf)Cl₂ (0.029 g, 0.04 mmol), CuI (0.035 g, 0.18 mmol), DIEA (0.077 g, 0.60 mmol), DMSO (2 mL). The reaction mixture was stirred at room temperature for 1 h. The reaction mixture was quenched by the addition of water (80 mL), extracted with EA (80 mL×2). The combined organic layers was washed with brine (20 mL), separated and concentrated under vacuum. The residue was purified by preparative HPLC (Mobile Phase A: water (10 mmoL/L ammonium hydroxide), Mobile Phase B: MeOH; Flow rate: 60 mL/min; Gradient: 50-80-100% B (2-30-60 min); 244 nm; RT: 35.560-37.110 min) to afford 3-fluoro-N-(2-(3-((2-methoxy-4-(methylsulfonyl)phenyl)amino)prop-1-yn-1-yl)-3-(2,2,2-trifluoroethyl)benzo[b]thiophen-7-yl)-1-methylpiperidin-4-amine (34) (0.053 g, 44.52% yield) as white solid. LCMS: m/z=602 [M+1]⁺

¹H NMR (400 MHz, MeOD) δ 7.49 (d, J=8.4 Hz, 1H), 7.33-7.23 (m, 2H), 7.18 (d, J=8.0 Hz, 1H), 6.94 (d, J=8.4 Hz, 1H), 6.83 (s, 1H), 4.41 (s, 2H), 3.99 (d, J=11.2 Hz, 1H), 3.95 (s, 3H), 3.72-3.63 (m, 2H), 3.11 (d, J=9.2 Hz, 1H), 3.07 (s, 3H), 2.90 (d, J=8.0 Hz, 1H), 2.60-2.44 (m, 1H), 2.37 (s, 3H), 2.35-2.25 (m, 1H), 2.06 (d, J=13.2 Hz, 1H), 1.98-1.84 (m, 1H).

### Example 35

### diethyl(4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)phosphine oxide (racemic) (35)

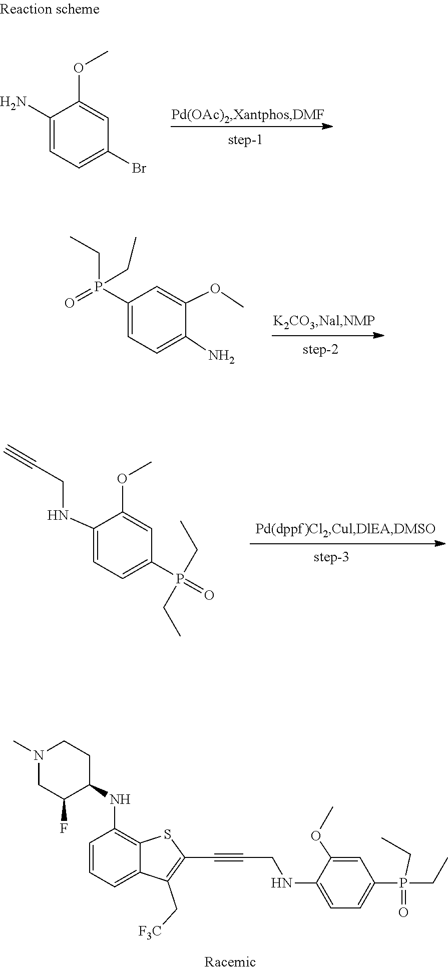

### Experimental Details

### Step 1. (4-amino-3-methoxyphenyl)diethylphosphine oxide

Into a 20-mL sealed tube purged and maintained with an inert atmosphere of nitrogen, was placed 4-bromo-2-methoxyaniline (0.981 g, 4.86 mmol), diethylphosphine oxide (1.039 g, 9.79 mmol), Pd(OAc)₂ (0.411 g, 1.83 mmol), Xantphos (0.381 g, 0.66 mmol), DIEA (1.841 g, 14.24 mmol), DMF (10 mL). The reaction mixture was stirred at 120° C. for 2 h. The reaction mixture was quenched by the addition of water (80 m L), extracted with EA (80 mL×2). The combined organic layers was washed with brine (40 mL), separated and concentrated under vacuum. The mixture was purified by C18 column eluted with ACN/water (v/v=1/8) to afford 0.775 g (70.24%) of (4-amino-3-methoxyphenyl)diethylphosphine oxide as colorless oil. LCMS: m/z=228 [M+1]⁺

### Step 2. diethyl(3-methoxy-4-(prop-2-yn-1-ylamino)phenyl)phosphine oxide

A mixture of (4-amino-3-methoxyphenyl)diethylphosphine oxide (0.423 g, 1.86 mmol), 3-bromoprop-1-yne (0.219 g, 1.84 mmol), NaI (0.227 g, 1.51 mmol) and K₂CO₃ (0.578 g, 4.18 mmol) in NMP (10 mL) was degassed and purged with N₂ for three times, The mixture was then stirred at 80° C. for 48 h. The reaction mixture was purified by C18 column eluted with ACN/water (v/v=1/3) to afford 0.172 g (34.83%) of di ethyl(3-methoxy-4-(prop-2-yn-1-ylamino)phenyl)phosphine oxide as colorless oil. LCMS: m/z=266 [M+1]⁺

### Step 3. diethyl(4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)phosphine oxide (racemic) (35)

Into a 8-mL sealed tube purged and maintained with an inert atmosphere of nitrogen, was placed (Z)-3-fluoro-N-(2-iodo-3-(2,2,2-trifluoroethyl)benzo[b]thiophen-7-yl)-1-methylpiperidin-4-amine (0.098 g, 0.21 mmol), diethyl(3-methoxy-4-(prop-2-yn-1-ylamino)phenyl)phosphine oxide (0.098 g, 0.37 mmol), Pd(dppf)Cl₂ (0.021 g, 0.03 mmol), CuI (0.048 g, 0.25 mmol), DIEA (0.099 g, 0.77 mmol), DMSO (1 mL). The reaction mixture was stirred at 50° C. for 1 h. The reaction mixture was quenched by the addition of water (20 mL), extracted with EA (20 mL×2). The combined organic layers was washed with brine (10 mL), separated and concentrated under vacuum. The mixture was purified by preparative HPLC (Mobile Phase A: water (10 mmoL/L ammonium hydroxide), Mobile Phase B: MeOH; Flow rate: 50 mL/min; Gradient: 35-65-95% B (2-30-60 min); 236 nm; RT: 38.125-42.751 min) to afford diethyl(4-((3-(7-(((Z)-3-fluoro-1-methylpiperidin-4-yl) amino)-3-(2,2,2-trifluoroethyl)benzo[b]thiophen-2-yl)prop-2-yn-1-yl)amino)-3-methoxyphenyl)phosphine oxide (racemic) (35). LCMS: m/z=610 [M+1]⁺

¹H NMR (400 MHz, DMSO) δ 7.26 (d, J=7.3 Hz, 1H), 7.25-7.12 (m, 2H), 7.08 (d, J=10.2 Hz, 1H), 6.86 (d, J=6.1 Hz, 1H), 6.78 (d, J=6.7 Hz, 1H), 6.07 (s, 1H), 5.15 (d, J=7.9 Hz, 1H), 4.80 (d, J=49.5 Hz, 1H), 4.35 (s, 2H), 3.84 (s, 3H), 3.82-3.71 (m, 2H), 3.71-3.55 (m, 1H), 3.13-2.95 (m, 1H), 2.80 (d, J=8.7 Hz, 1H), 2.27 (d, J=12.5 Hz, 1H), 2.18 (s, 3H), 2.13-2.04 (m, 1H), 1.99-1.68 (m, 6H), 1.05-0.82 (m, 6H).

### Example 36

### 1-(2-fluoroethyl)-N-(2-(3-((2-methoxy-4-(methylsulfonyl)phenyl)amino)prop-1-yn-1-yl)-3-(2,2,2-trifluoroethyl) benzo[b]thiophen-7-yl)piperidin-4-amine (36)

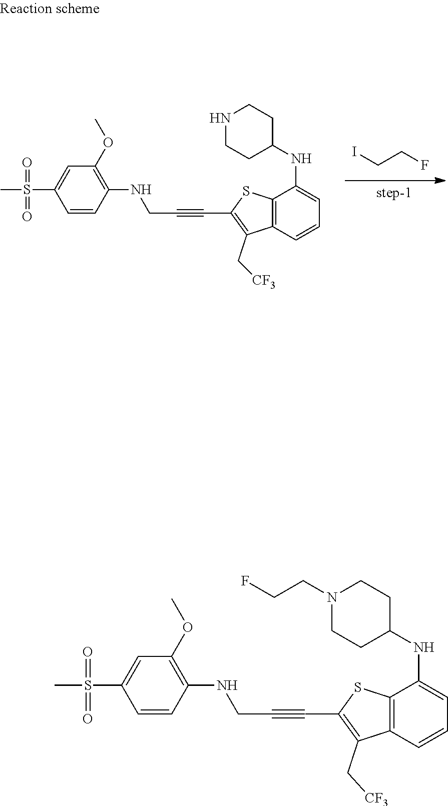

### Experimental Details

### Step 1. 1-(2-fluoroethyl)-N-(2-(3-((2-methoxy-4-(methylsulfonyl)phenyl)amino)prop-1-yn-1-yl)-3-(2,2,2-trifluoroethyl)benzo[b]thiophen-7-yl)piperidin-4-amine (36)

Into a 4-mL sealed tube and maintained with an inert atmosphere of nitrogen, was placed N-(2-(3-((2-meth oxy-4-(methylsulfonyl)phenyl)amino)prop-1-yn-1-yl)-3-(2,2,2-trifluoroethyl)benzo[b]thiophen-7-yl)piperidin-4-amine (0.040 g, 72.51 umol), 1-fluoro-2-iodoethane (0.025 g, 143.71 umol), K₂CO₃ (0.063 g, 455.86 umol), acetonitrile (1 mL). The reaction mixture was stirred at 50° C. overnight. The resulting solution was added to water (10 mL). The resulting solution was extracted with EA (2×10 mL), the organic layers combined, dried over anhydrous Na₂SO₄, the residue was concentrated under vacuum. The crude product purified by pre-HPLC with MeCN/H₂O (0.1% ammonium hydroxide), Flow rate: 70 mL/min; Gradient: 40-75-100% B (2-30-60 min); 263 nm; RT: 33.540-34.450 min. This resulted in 0.018 g (41.54% yield) of 1-(2-fluoroethyl)-N-(2-(3-((2-methoxy-4-(methylsulfonyl)phenyl)amino)prop-1-yn-1-yl)-3-(2,2,2-trifluoroethyl)benzo[b]thiophen-7-yl) piperidin-4-amine (36) as white solid. LCMS: m/z=598 [M+1]⁺

¹H NMR (400 MHz, DMSO) δ 7.40 (dd, J=8.3, 1.6 Hz, 1H), 7.35-7.20 (m, 2H), 7.14 (d, J=8.0 Hz, 1H), 6.89 (d, J=8.4 Hz, 1H), 6.67 (d, J=7.8 Hz, 1H), 6.52 (t, J=6.2 Hz, 1H), 5.32 (d, J=8.0 Hz, 1H), 4.59 (t, J=4.8 Hz, 1H), 4.47 (t, J=4.9 Hz, 1H), 4.38 (d, J=6.2 Hz, 2H), 3.90 (s, 3H), 3.86-3.71 (m, 2H), 3.10 (s, 3H), 2.89 (d, J=11.8 Hz, 2H), 2.65 (t, J=4.9 Hz, 1H), 2.58 (t, J=4.9 Hz, 1H), 2.14 (t, J=11.1 Hz, 2H), 1.90 (d, J=11.4 Hz, 2H), 1.54 (dd, J=20.8, 11.3 Hz, 2H), 1.24 (s, 1H).

### Example 37

### N-(2-(3-((2-methoxy-4-(methylsulfonyl)phenyl)amino)prop-1-yn-1-yl)-3-(2,2,2-trifluoroethyl)benzo[b]thiophen-7-yl)-1-(2,2,2-trifluoroethyl)piperidin-4-amine (37)

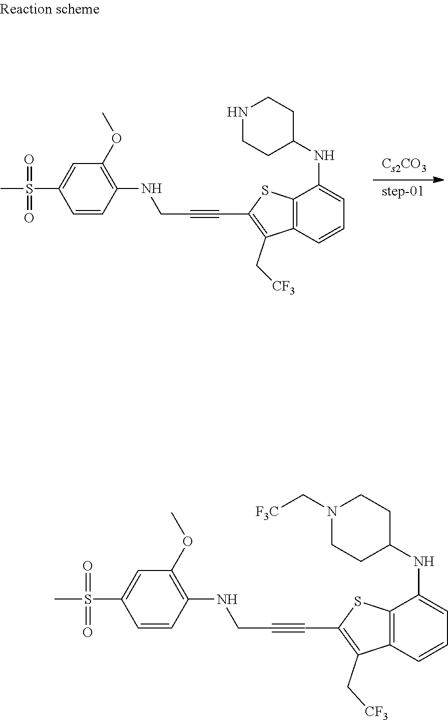 [SMILES: COc1cc(S(C)(=O)=O)ccc1NCC#Cc1sc2c(NC3CCNCC3)cccc2c1CC(F)(F)F ; COc1cc(S(C)(=O)=O)ccc1NCC#Cc1sc2c(NC3CCN(CC(F)(F)F)CC3)cccc2c1CC(F)(F)F]

### Step 1. N-(2-(3-((2-methoxy-4-(methylsulfonyl)phenyl)amino)prop-1-yn-1-yl)-3-(2,2,2-trifluoroethyl)benzo[b]thiophen-7-yl)-1-(2,2,2-trifluoroethyl)piperidin-4-amine (37)

Into a 4-mL sealed tube and maintained with an inert atmosphere of nitrogen, was placed N-(2-(3-((2-meth oxy-4-(methylsulfonyl)phenyl)amino)prop-1-yn-1-yl)-3-(2,2,2-trifluoroethyl)benzo[b]thiophen-7-yl)piperidin-4-amine (0.023 g, 41.69 μmol), 2,2,2-trifluoroethyl trifluoromethanesulfonate (0.017 g, 73.24 μmol), Cs₂CO₃ (0.028 g, 85.94 μmol), acetonitrile (1 mL). The reaction mixture was stirred at room temperature for 2 h. The resulting solution was added to water (10 mL). The resulting solution was extracted with EA (2×10 mL), the organic layers combined, dried over anhydrous Na₂SO₄, the residue was concentrated under vacuum. The crude product purified by pre-HPLC with MeOH/H₂O (0.1% ammonium hydroxide), Flow rate: 40 mL/min; Gradient: 45-75-100% B (2-30-60 min); 270 nm; RT: 38.379-40.311 min. This resulted in 0.016 g (60.56% yield) of N-(2-(3-((2-methoxy-4-(methylsulfonyl)phenyl)amino)prop-1-yn-1-yl)-3-(2,2,2-trifluoroethyl)benzo[b]thiophen-7-yl)-1-(2,2,2-trifluoroethyl)piperidin-4-amine (37) as white solid. LCMS: m/z=634 [M+1]⁺

¹H NMR (400 MHz, DMSO) δ 7.39 (d, J=6.9 Hz, 1H), 7.25 (t, J=7.8 Hz, 2H), 7.15 (d, J=7.9 Hz, 1H), 6.89 (d, J=8.4 Hz, 1H), 6.68 (d, J=7.8 Hz, 1H), 6.52 (t, J=6.0 Hz, 1H), 5.30 (d, J=8.0 Hz, 1H), 4.38 (d, J=6.1 Hz, 2H), 3.90 (s, 2H), 3.80 (dd, J=21.9, 10.9 Hz, 2H), 3.64 (d, J=12.7 Hz, 2H), 3.17 (dd, J=20.5, 10.3 Hz, 3H), 3.10 (s, 4H), 2.93 (d, J=11.6 Hz, 2H), 1.89 (d, J=11.1 Hz, 2H), 1.55 (dd, J=20.5, 11.4 Hz, 2H).

### Example 38

### N-(2-(3-((2-methoxy-4-(methylsulfonyl)phenyl)amino)prop-1-yn-1-yl)-3-(2,2,2-trifluoroethyl)benzo[b]thiophen-7-yl)piperidin-4-amine (38)

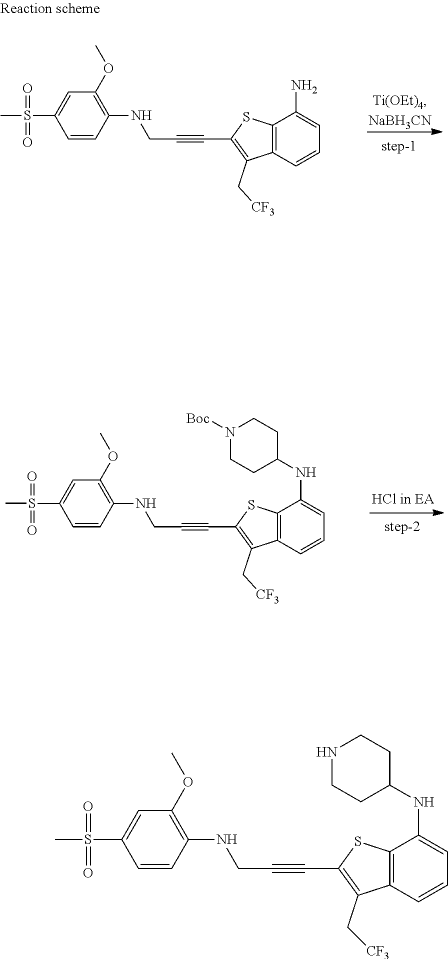 [SMILES: COc1cc(S(C)(=O)=O)ccc1NCC#Cc1sc2c(N)cccc2c1CC(F)(F)F ; COc1cc(S(C)(=O)=O)ccc1NCC#Cc1sc2c(NC3CCN(C(=O)OC(C)(C)C)CC3)cccc2c1CC(F)(F)F ; COc1cc(S(C)(=O)=O)ccc1NCC#Cc1sc2c(NC3CCNCC3)cccc2c1CC(F)(F)F]

### Experimental Details

### Step 1. tert-butyl 4-((2-(3-((2-methoxy-4-(methylsulfonyl)phenyl)amino)prop-1-yn-1-yl)-3-(2,2,2-trifluoroethyl)benzo[b]thiophen-7-yl)amino)piperidine-1-carboxylate

Into a 40-mL sealed tube and maintained with an inert atmosphere of nitrogen, was placed 2-(3-((2-methoxy-4-(methylsulfonyl)phenyl)amino)prop-1-yn-1-yl)-3-(2,2,2-trifluoroethyl)benzo[b]thiophen-7-amine (0.303 g, 0.65 mmol), tert-butyl 4-oxopiperidine-1-carboxylate (0.646 g, 3.24 mmol), titanium ethoxide (0.754 g, 3.29 mmol), toluene (4 mL). The reaction mixture was stirred at 110° C. for 2 h. Sodium cyanoborohydride (0.170 g, 2.75 mmol) was added to the reaction and stirred at room temperature for 3 h. The reaction was concentrated under vacuum and the residue was applied onto a silica gel column eluted with EA/hexane (v/v=1/1). This resulted in 0.348 g (82.56% yield) of tert-butyl 4-((2-(3-((2-methoxy-4-(methylsulfonyl)phenyl)amino)prop-1-yn-1-yl)-3-(2,2,2-trifluoroethyl)benzo[b]thiophen-7-yl)amino)piperidine-1-carboxylate as yellow solid. LCMS: m/z=652 [M+1]⁺

### Step 2. N-(2-(3-((2-methoxy-4-(methylsulfonyl)phenyl)amino)prop-1-yn-1-yl)-3-(2,2,2-trifluoroethyl)benzo[b]thiophen-7-yl)piperidin-4-amine (38)

Into a 25-mL round-bottom flask purged and maintained with an inert atmosphere of nitrogen, was placed tert-butyl 4-((2-(3-((2-methoxy-4-(methylsulfonyl)phenyl)amino)prop-1-yn-1-yl)-3-(2,2,2-trifluoroethyl)benzo[b]thiophen-7-yl)amino)piperidine-1-carboxylate (0.336 g, 0.52 mmol) and EA (3 mL). The reaction mixture was stirred at room temperature, then HCl in EA (3 mL, 4 M) was added to the reaction and stirred at room temperature for 2 h. The resulting solution was added to saturated sodium bicarbonate aqueous solution (20 mL). The resulting solution was extracted with EA (2×30 mL), the organic layers combined, dried over anhydrous Na₂SO₄, the residue was concentrated under vacuum. A quarter of the resulting crude product was further purified by pre-HPLC with MeOH/H₂O (0.1% ammonium hydroxide), Flow rate: 25 m/min; Gradient: 40-70-100% B (2-30-60 min); 220 nm; RT: 32.890-37.835 min. This resulted in 0.028 g (49.30% yield) of N-(2-(3-((2-methoxy-4-(methylsulfonyl)phenyl)amino)prop-1-yn-1-yl)-3-(2,2,2-trifluoroethyl)benzo[b]thio phen-7-yl)piperidin-4-amine (38) as yellow solid. LCMS: m/z=552 [M+1]⁺

¹H NMR (400 MHz, DMSO) δ 7.39 (d, J=8.3 Hz, 1H), 7.25 (dd, J=7.7, 4.8 Hz, 2H), 7.14 (d, J=7.9 Hz, 1H), 6.89 (d, J=8.4 Hz, 1H), 6.65 (t, J=16.6 Hz, 1H), 6.52 (t, J=6.3 Hz, 1H), 5.33 (d, J=7.9 Hz, 1H), 4.38 (d, J=6.2 Hz, 2H), 3.90 (s, 3H), 3.80 (dd, J=22.0, 11.0 Hz, 2H), 3.42 (s, 2H), 3.10 (s, 3H), 2.97 (t, J=16.5 Hz, 2H), 2.68-2.52 (m, 2H), 1.88 (d, J=11.1 Hz, 2H), 1.39 (dd, J=20.0, 11.3 Hz, 2H).

### Example 39

### Cis-N-(4-(2-oxa-6-azaspiro[3.3]heptan-6-yl)cyclohexyl)-2-(3-((2-methoxy-4-(methylsulfonyl)phenyl)amino)prop-1-yn-1-yl)-1-(2,2,2-trifluoroethyl)-1H-indol-4-amine (39) and trans-N-((1R,4R)-4-(2-oxa-6-azaspiro[3.3]heptan-6-yl)cyclohexyl)-2-(3-((2-methoxy-4-(methylsulfonyl)phenyl)amino)prop-1-yn-1-yl)-1-(2,2,2-trifluoroethyl)-1H-indol-4-amine (39b)

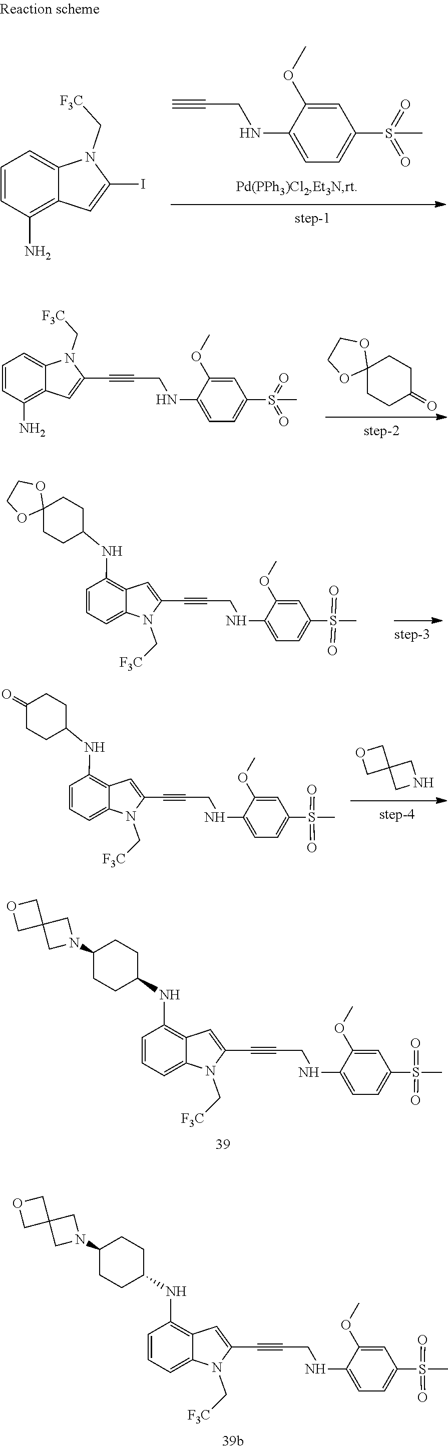

### Step 1. Synthesis of 2-(3-((2-methoxy-4-(methylsulfonyl)phenyl)amino)prop-1-yn-1-yl)-1-(2,2,2-trifluoroethyl)-1H-indol-4-amine

Into a 50 mL flask was added 2-iodo-1-(2,2,2-trifluoroethyl)-1H-indol-4-amine (0.620 g, 1.82 mmol), 4-(methylsulfonyl)-N-(prop-2-yn-1-yl)aniline (0.457 g, 1.90 mmol), bis(triphenylphosphine)palladium(II) chloride (0.240 g, 339.97 μmol), N,N-diisopropylethylamine (0.458 g, 3.54 mmol), CuI (0.177 g, 929.37 μmol), methyl sulfoxide (20 mL). The reaction mixture was stirred at rt for 2 h. The reaction was quenched with H₂O (30 mL), extracted with EA (50 mL×3) and concentrated under vacuum. The crude was purified by silica gel column eluted with EA/Hexane (v/v=1/1) to afford 2-(3-((2-methoxy-4-(methylsulfonyl)phenyl)amino) prop-1-yn-1-yl)-1-(2,2,2-trifluoroethyl)-1H-indol-4-amine (0.545 g, 66.21%) as yellow solid. LCMS: m/z=452 [M+1]⁺.

### Step 2. 2-(3-((2-methoxy-4-(methylsulfonyl)phenyl)amino)prop-1-yn-1-yl)-N-(1,4-dioxaspiro[4.5]decan-8-yl)-1-(2,2,2-trifluoroethyl)-1H-indol-4-amine

Into a 50 mL flask was placed 2-(3-((2-methoxy-4-(methylsulfonyl)phenyl)amino)prop-1-yn-1-yl)-1-(2,2,2-trifluoroethyl)-1H-indol-4-amine (0.545 g, 1.20 mmol), 1,4-dioxaspiro[4.5]decan-8-one (0.574 g, 3.67 mmol), titanium ethoxide, toluene (10 mL). The reaction was stirred at 100° C. for 2 h before cooled to RT. The reaction mixture was concentrated under vacuum. The residue was added EtOH (5 mL), sodium cyanoboronhydride (0.392 g, 9.1427 mmol). The reaction was stirred at RT for 2 h. The reaction was quenched with H₂O (10 mL), extracted with EA (3×30 mL), concentrated under vacuum. The crude was purified by silica gel column eluted with EA/Hexane (v/v=1/1) to afford 2-(3-((2-methoxy-4-(methylsulfonyl)phenyl)amino)prop-1-yn-1-yl)-N-(1,4-dioxaspiro[4.5]decan-8-yl)-1-(2,2,2-trifluoroethyl)-1H-indol-4-amine (0.626 g, 87.64%) as yellow solid. LCMS: m/z=592 [M+1]⁺.

### Step 3. 4-((2-(3-((2-methoxy-4-(methylsulfonyl)phenyl)amino)prop-1-yn-1-yl)-1-(2,2,2-trifluoroethyl)-1H-indol-4-yl)amino)cyclohexan-1-one

Into a 50 mL flask was placed 2-(3-((2-methoxy-4-(methylsulfonyl)phenyl)amino)prop-1-yn-1-yl)-N-(1,4-dioxa spiro[4.5]decan-8-yl)-1-(2,2,2-trifluoroethyl)-1H-indol-4-amine (0.731 g, 1.23 mmol), 4-methylbenzenesulfonic acid hydrate (2.002 g, 10.52 mmol), acetonitrile (10 mL), water (5 mL). The reaction was stirred at RT for 1 h under N₂ atmosphere. The reaction was quenched with H₂O (30 mL), extracted with EA (3×50 m L), concentrated under vacuum. The crude was purified by silica gel column eluted with EA/hexane (v/v=3/1) to afford 4-((2-(3-((2-methoxy-4-(methylsulfonyl)phenyl)amino)prop-1-yn-1-yl)-1-(2,2,2-trifluoroethyl)-1H-indol-4-yl)amino)cyclohexan-1-one (0.402 g, 59.41%) as yellow solid. LCMS: m/z=548 [M+1]⁺.

### Step 4. cis-N-(4-(2-oxa-6-azaspiro[3.3]heptan-6-yl)cyclohexyl)-2-(3-((2-methoxy-4-(methylsulfonyl)phenyl)amino)prop-1-yn-1-yl)-1-(2,2,2-trifluoroethyl)-1H-indol-4-amine (39) and trans-N-(4-(2-oxa-6-azaspiro[3.3]heptan-6-yl)cyclohexyl)-2-(3-((2-methoxy-4-(methylsulfonyl)phenyl)amino)prop-1-yn-1-yl)-1-(2,2,2-trifluoroethyl)-1H-indol-4-amine (39b)

Into a 25 mL flask was placed 4-((2-(3-((2-methoxy-4-(methylsulfonyl)phenyl)amino)prop-1-yn-1-yl)-1-(2,2,2-trifluoroethyl)-1H-indol-4-yl)amino)cyclohexan-1-one (0.219 g, 399.93 μmol), 2-oxa-6-azaspiro[3.3]heptane (0.145 g, 1.4627 mmol), acetic acid (0.264 g, 4.39 mmol), MeOH (5 mL). The reaction was stirred at rt for 12 h, sodium cyanoboronhydride (0.392 g, 9.1427 mmol) was added to above mixture. The reaction was stirred at RT for 2 h. The reaction was quenched with H₂O (10 mL), extracted with EA (3×30 mL), concentrated under vacuum. The mixture was purified by preparative HPLC (Mobile Phase A: water (10 mmoL/L ammonium hydroxide), Mobile Phase B: CAN; Flow rate: 70 mL/min; Gradient: 30-60-60% B (2-32-60 min), 248 nm; RT: 30.513-32.468 and RT: 34.848-36.835 min) to afford cis-N-(4-(2-oxa-6-azaspiro[3.3]heptan-6-yl)cyclohexyl)-2-(3-((2-methoxy-4-(methylsulfonyl)phenyl)amino)prop-1-yn-1-yl)-1-(2,2,2-trifluoroethyl)-1H-indol-4-amine (39) (0.027 g, 10.70%) as off-white solid. LCMS: m/z=631 [M+1]⁺.

¹H NMR (400 MHz, DMSO) δ 7.38 (d, J=8.3 Hz, 1H), 7.25 (s, 1H), 7.06 (s, 1H), 6.98 (t, J=7.9 Hz, 1H), 6.88 (d, J=8.4 Hz, 1H), 6.66 (d, J=8.2 Hz, 1H), 6.49 (t, J=6.0 Hz, 1H), 6.12 (d, J=7.8 Hz, 1H), 5.44 (d, J=8.0 Hz, 1H), 4.91 (q, J=8.8 Hz, 2H), 4.58 (s, 4H), 4.35 (d, J=6.1 Hz, 2H), 3.89 (s, 3H), 3.21 (s, 5H), 3.09 (s, 3H), 1.96 (d, J=11.1 Hz, 2H), 1.86 (t, J=10.5 Hz, 1H), 1.71 (d, J=11.3 Hz, 2H), 1.19 (dd, J=24.1, 11.5 Hz, 2H), 0.99 (dd, J=23.4, 10.9 Hz, 2H).

And trans-N-(4-(2-oxa-6-azaspiro[3.3]heptan-6-yl)cyclohexyl)-2-(3-((2-methoxy-4-(methylsulfonyl)phenyl)amino) prop-1-yn-1-yl)-1-(2,2,2-trifluoroethyl)-1H-indol-4-amine (39b) (0.023 g, 9.11%) as off-white solid. LCMS: m/z=631 [M+1]⁺.

¹H NMR (400 MHz, DMSO) δ 7.38 (d, J=8.2 Hz, 1H), 7.25 (s, 1H), 7.12 (s, 1H), 6.97 (t, J=7.9 Hz, 1H), 6.89 (d, J=8.4 Hz, 1H), 6.64 (d, J=8.1 Hz, 1H), 6.49 (t, J=6.0 Hz, 1H), 6.12 (d, J=7.8 Hz, 1H), 5.48 (d, J=8.0 Hz, 1H), 4.90 (q, J=8.9 Hz, 2H), 4.59 (s, 4H), 4.35 (d, J=6.0 Hz, 2H), 3.89 (s, 3H), 3.20 (s, 4H), 3.09 (s, 3H), 2.09 (d, J=12.5 Hz, 1H), 1.69-1.46 (m, 6H), 1.39 (s, 2H), 1.23 (s, 1H).

### Example 40

### 4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-methoxybenzenesulfonamide (racemic) (40)

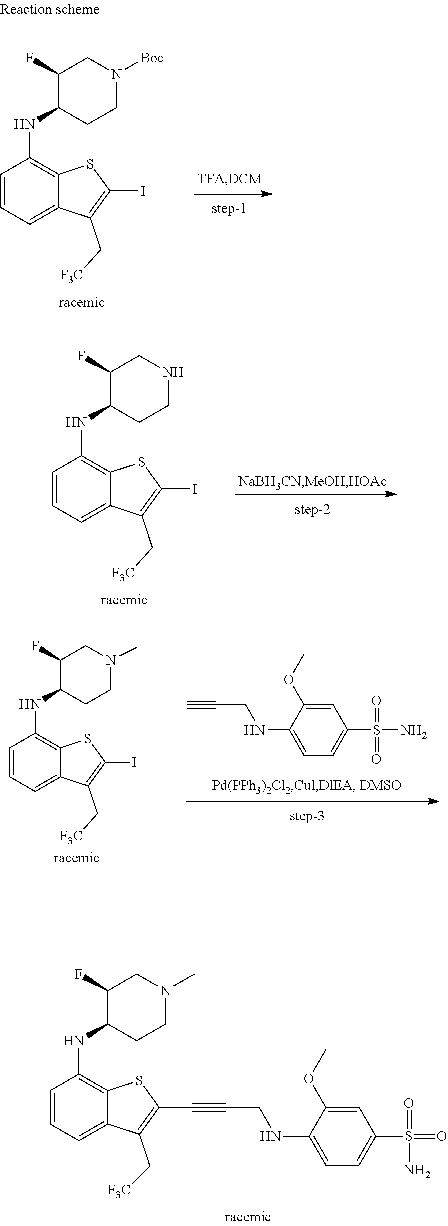 [SMILES: CC(C)(C)OC(=O)N1CC[C@@H](Nc2cccc3c(CC(F)(F)F)c(I)sc23)[C@@H](F)C1 ; F[C@H]1CNCC[C@H]1Nc1cccc2c(CC(F)(F)F)c(I)sc12 ; CN1CC[C@@H](Nc2cccc3c(CC(F)(F)F)c(I)sc23)[C@@H](F)C1 ; C#CCNc1ccc(S(N)(=O)=O)cc1OC ; COc1cc(S(N)(=O)=O)ccc1NCC#Cc1sc2c(N[C@@H]3CCN(C)C[C@@H]3F)cccc2c1CC(F)(F)F]

### Experimental Details

### Step 1. (Z)-3-fluoro-N-(2-iodo-3-(2,2,2-trifluoroethyl)benzo[b]thiophen-7-yl)piperidin-4-amine (racemic)

Into a 500 mL round-bottom flask was placed tert-butyl tert-butyl (Z)-3-fluoro-4-((2-iodo-3-(2,2,2-trifluoroethyl)benzo[b]thiophen-7-yl)amino)piperidine-1-carboxylate (racemic) (16.02 g, 28.69 mmol), DCM (150 mL), T FA (50 mL). The reaction was stirred at RT for 1 h. The reaction was quenched with saturated aqueous solution of NaHCO₃ (100 mL), extracted with EA (3×100 mL). The combined organic layers was washed with brine (200 mL), dried over anhydrous sodium sulfate, filtered and concentrated under vacuum. This resulted in 14.04 g (crude) of (Z)-3-fluoro-N-(2-iodo-3-(2,2,2-trifluoroethyl)benzo[b]thiophen-7-yl)piperidin-4-amine (racemic) as off-white solid. LCMS: m/z=459 [M+1]⁺.

### Step 2. (Z)-3-fluoro-N-(2-iodo-3-(2,2,2-trifluoroethyl)benzo[b]thiophen-7-yl)-1-methylpiperidin-4-amine (rac emic)

Into a 500 mL round-bottom flask was placed (Z)-3-fluoro-N-(2-iodo-3-(2,2,2-trifluoroethyl)benzo[b]thiophen-7-yl)piperidin-4-amine (racemic) (13.962 g, 30.47 mmol), polyoxymethylene (1.575 g, 52.45 mmol), sodium cyanoboronhydride (11.745 g, 189.94 mmol), MeOH (150 mL), HOAc (30 mL). The reaction mixture was stirred overnight at RT. The reaction was quenched with saturated aqueous solution of NaHCO₃ (100 mL), extracted with EA (3×100 mL), The combined organic layers was washed with brine (50 mL), dried over anhydrous sodium sulfate, filtered and concentrated under vacuum. The crude was purified by silica gel column eluted with MeOH/DCM (v/v=1/20). This resulted in 8.513 g (59.43% yield) of (Z)-3-fluoro-N-(2-iodo-3-(2,2,2-trifluoroethyl)benzo[b]thiophen-7-yl)-1-methylpiperidin-4-amine (racemic) as off-white solid. LC MS: m/z=473 [M+1]⁺.

### Step 3. 4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-methoxybenzenesulfonamide (racemic) (40)

Into a 25-mL round-bottom flask purged and maintained with an inert atmosphere of nitrogen was placed (Z)-3-fluoro-N-(2-iodo-3-(2,2,2-trifluoroethyl)benzo[b]thiophen-7-yl)-1-methylpiperidin-4-amine (racemic) (0.119 g, 251.97 μmol), 3-methoxy-4-(prop-2-yn-1-ylamino)benzenesulfonamide (0.094 g, 391.21 μmol), Bis(triphenylphosphine)palladium(II) chloride (0.051 g, 72.25 μmol), CuI (0.044 g, 231.03 μmol), DIEA (0.159 g, 1.23 mmol), methyl sulfoxide (5 mL). The reaction was stirred for 2 h at 60° C. under nitrogen atmosphere. The reaction was quenched with water (20 mL), extracted with EA (30 mL). The combined organic layers was washed with brine (30 mL), dried over anhydrous sodium sulfate, filtered and concentrated under vacuum. The mixture was purified by preparative HPLC (Mobile Phase A: water (10 mmoL/L ammonium hydr oxide), Mobile Phase B: MeOH; Flow rate: 70 mL/min; Gradient: 50-90-100% B (2-30-40 min); 270 nm; RT: 26.258-28.090 min) to provide the desired product. This resulted in 0.072 g (48.88% yield) of 4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-methoxybenzenesulfonamide (racemic) (40) as yellow solid. LCMS: m/z=585 [M+1]⁺.

¹H NMR (400 MHz, Methanol-d₄) δ 7.50-7.43 (m, 1H), 7.36-7.26 (m, 2H), 7.20 (d, J=8.0 Hz, 1H), 6.86 (d, J=8.4 Hz, 1H), 6.78 (d, J=7.6 Hz, 1H), 4.38 (s, 2H), 3.93 (s, 3H), 3.80-3.63 (m, 3H), 3.22 (d, J=23.6 Hz, 2H), 2.95 (d, J=11.6 Hz, 1H), 2.33-2.12 (m, 5H), 2.02-1.90 (m, 2H).

### Example 41

### 4-((3-(7-(((3R,4S)-3-fluoro-1-methylpiperidin-4-yl)amino)-3-(2,2,2-trifluoroethyl)benzo[b]thiophen-2-yl)prop-2-yn-1-yl)amino)-3-methoxy-N-methylbenzamide (racemic) (41)

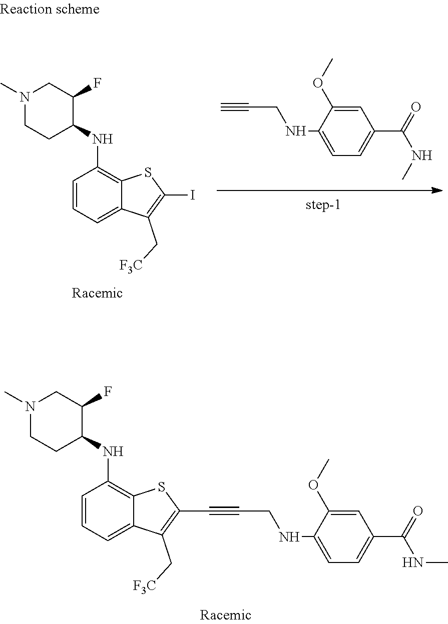

### Experimental Details

### Step 1. 4-((3-(7-(((3R,4S)-3-fluoro-1-methylpiperidin-4-yl)amino)-3-(2,2,2-trifluoroethyl)benzo[b]thiophen-2-yl)prop-2-yn-1-yl)amino)-3-methoxy-N-methylbenzamide (racemic) (41)

Into a 8 mL vial purged and maintained with an inert atmosphere of nitrogen, was placed (3R,4S)-3-fluoro-N-(2-iodo-3-(2,2,2-trifluoroethyl)benzo[b]thiophen-7-yl)-1- methylpiperidin-4-amine (racemic) (0.137 g, 290.08 μmol), cuprous iodide (0.015 g, 78.76 μmol), 3-methoxy-N-methyl-4-(prop-2-yn-1-ylamino)benzamide (0.119 g, 545.24 μmol), bis(triphenylphosphine)palladium(II) chloride (0.023 g, 32.5813 μmol), triethylamine (0.119 g, 1.18 mmol), methyl sulfoxide (4 mL). The reaction mixture was stirred at room temperature for 3 h. The reaction was quenched by the addition of water (10 mL) and extracted with EA (3×10 mL). The organic layers was combined, washed with brine (10 mL), dried over anhydrous Na₂SO₄ and concentrated un der vacuum. The mixture was purified by preparative HPLC (Mobile Phase A: water (10 mmoL/L ammonium hydroxide), Mobile Phase B: ACN; Flow rate: 70 mL/min; Gradient: 35-65-90% B (2-30-60 min); 269 nm; RT: 32.043-33.407 min). This resulted in 0.075 g (45.95% yield) of 4-((3-(7-(((3R,4S)-3-fluoro-1-methylpiperidin-4-yl)amino)-3-(2,2,2-trifluoroethyl)benzo[b]thiophen-2-yl)prop-2-yn-1-yl)amino)-3-methoxy-N-methyl benzamide (racemic) (41) as white solid. LCMS: m/z=563 [M+1]⁺.

¹H NMR (400 MHz, DMSO) δ 8.11 (s, 1H), 7.42 (d, J=8.3 Hz, 1H), 7.35 (s, 1H), 7.26 (d, J=7.5 Hz, 1H), 7.20 (d, J=8.3 Hz, 1H), 6.76 (t, J=7.9 Hz, 2H), 6.01 (s, 1H), 5.22-5.14 (m, 1H), 4.90-4.84 (m, 1H), 4.80-4.70 (m, 1H), 4.33 (d, J=6.2 Hz, 2H), 3.89-3.74 (m, 5H), 2.75 (d, J=4.2 Hz, 3H), 2.20 (s, 3H), 1.91 (s, 3H), 1.73 (s, 2H), 1.23 (s, 1H).

### Example 42

### (Z)-3-fluoro-N-(2-(3-((2-methoxy-4-(methylsulfonyl)phenyl)amino)prop-1-yn-1-yl)-3-(2,2,2-trifluoroethyl)benzo[b]thiophen-7-yl)-N,1-dimethylpiperidin-4-amine (racemic) (42)

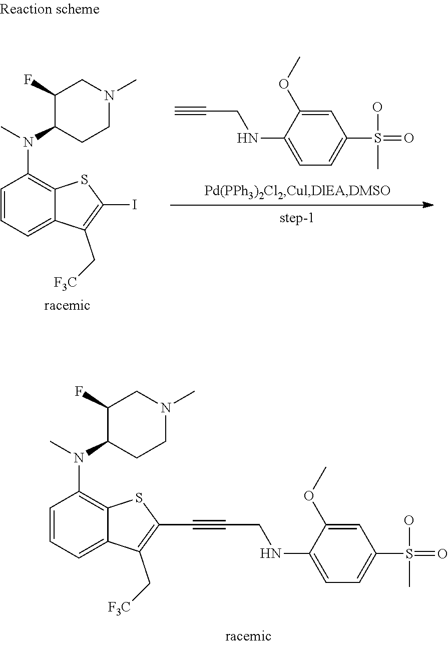

### Experimental Details

### Step 1. (Z)-3-fluoro-N-(2-(3-((2-methoxy-4-(methylsulfonyl)phenyl)amino)prop-1-yn-1-yl)-3-(2,2,2-trifluoroethyl)benzo[b]thiophen-7-yl)-N,1-dimethylpiperidin-4-amine (racemic) (42)

Into a 40 mL vial was placed 2-methoxy-4-(methylsulfonyl)-N-(prop-2-yn-1-yl)aniline (0.296 g, 1.24 mmol), (Z)-3-fluoro-N-(2-iodo-3-(2,2,2-trifluoroethyl)benzo[b]thiophen-7-yl)-N,1-dimethylpiperidin-4-amine (racemic) (0.281 g, 578.19 μmol), bis(triphenylphosphine)palladium(II) chloride (0.157 g, 222.40 μmol), CuI (0.145 g, 761.35 μmol), DIEA (0.745 g, 5.76 mmol), Methyl sulfoxide (20 mL). The reaction was stirred at 60° C. for 3 h under nitrogen atmosphere. The reaction was quenched with water (30 mL), extracted with EA (3×20 mL). The organic layers was combined, washed with brine (50 mL), dried over anhydrous sodium sulfate, filtered and concentrated under vacuum. The mixture was purified by preparative HPLC (Mobile Phase A: water (10 mmoL/L ammonium hydroxide), Mobile Phase B: MeOH; Flow rate: 70 mL/min; Gradient: 55-80-100% B (2-30-60 min); 269 nm; RT: 36.753-38.278 min) to provide the desired product. This resulted in 0.015 g (4.35% yield) of (Z)-3-fluoro-N-(2-(3-((2-methoxy-4-(methylsulfonyl)phenyl)amino)prop-1-yn-1-yl)-3-(2,2,2-trifluoroethyl)benzo[b]thiophen-7-yl)-N,1-dimethylpiperidin-4-amine (racemic) (42) as off-white sol id. LCMS: m/z=598 [M+1]⁺.

¹H NMR (400 MHz, DMSO-d₆) δ 7.58 (d, J=8.0 Hz, 1H), 7.47-7.37 (m, 2H), 7.29-7.21 (m, 2H), 6.89 (d, J=8.4 Hz, 1H), 6.50 (t, J=6.4 Hz, 1H), 4.84 (d, J=50.0 Hz, 1H), 4.39 (d, J=6.4 Hz, 2H), 3.94-3.79 (m, 5H), 3.28-3.20 (m, 1H), 3.09 (s, 3H), 2.96 (t, J=12.4 Hz, 1H), 2.83 (s, 4H), 2.10 (d, J=21.2 Hz, 4H), 2.06-1.89 (m, 2H), 1.52 (d, J=12.0 Hz, 1H).

### Example 43

### (4-((3-(7-((3,3-difluoro-1-methylpiperidin-4-yl)amino)-3-(2,2,2-trifluoroethyl)benzo[b]thiophen-2-yl)prop-2-yn-1-yl)amino)-3-methoxyphenyl)dimethylphosphine oxide (43)

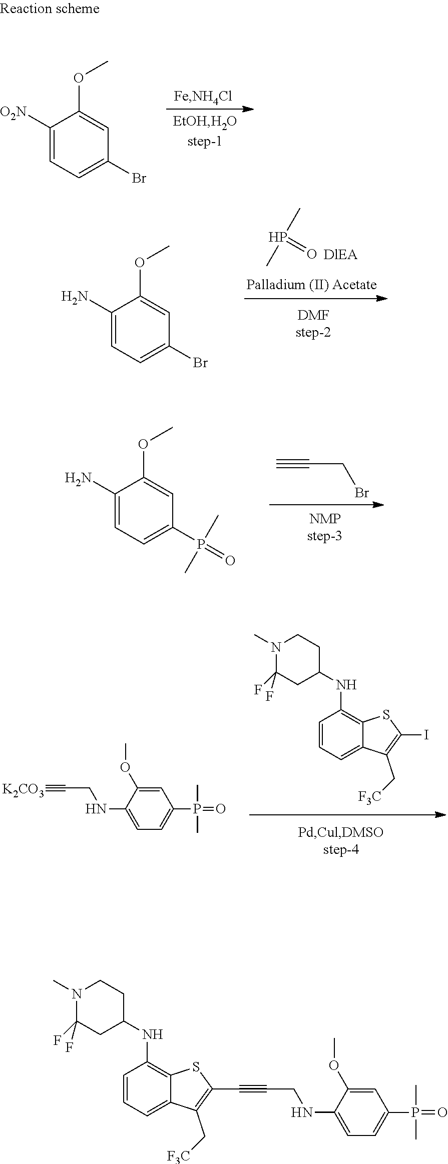

### Experimental Details

### Step 1 4-bromo-2-methoxyaniline

Into a 500 mL 3-necked flask was placed 4-bromo-2-methoxy-1-nitrobenzene (15.75 g, 67.87 mmol), iron (28.61 g, 512.31 mmol), NH₄Cl (28.40 g, 530.92 mmol), EtOH (150 mL), water (15 mL). The reaction w as stirred at RT for 3 h. The reaction mixture was filtered through celite pad and the filter cake was washed with methanol (2×200 mL). The filtrate was concentrated under reduced pressure to afford the product. The crude product was purified by silica gel column eluted with EA/hexane (v/v=1/2). This resulted in 13.82 g (68.39 mmol, 100.00% yield) of 4-bromo-2-methoxyaniline as yellow oil. LCMS: m/z=202 [M+1]⁺.

### Step 2 (4-amino-3-methoxyphenyl)dimethylphosphine oxide

Into a 250 mL 3-necked flask was placed 4-bromo-2-methoxyaniline (5.055 g, 25.01 mmol), Palladium (II) acetate, (0.868 g, 3.87 mmol), dimethylbisdiphenylphosphinoxanthene (2.143 g, 3.70 mmol), DIEA (6.442 g, 49.84 mmol), The reaction was stirred under nitrogen atmosphere at 130° C. for 0.5 h. Then dimethylphosphine oxide (4.83 g, 61.88 mmol) was added at 130° C. The reaction was stirred under nitrogen atmosphere at 130° C. for 3 h The reaction mixture was purified by C18 column eluted with ACN/H₂O (v/v=1/9), filtered and concentrated under vacuum. This resulted in 4.798 g, (24.08 mmol, 96.27% yield) of (4-amino-3-methoxyphenyl)dimethylphosphine oxide as brown oil. LCMS: m/z=200 [M+1]⁺.

### Step 3 (3-methoxy-4-(prop-2-yn-1-ylamino)phenyl)dimethylphosphine oxide

Into a 250 mL 3-necked flask was placed (4-amino-3-methoxyphenyl)dimethylphosphine oxide (2.059 g, 10.33 mmol), 3-bromoprop-1-yne (1.693 g, 14.23 mmol), K₂CO₃ (3.305 g, 23.91 mmol), KI (1.877 g, 11.30 mmol) NMP (25 mL). The reaction mixture was stirred under nitrogen at 80° C. for 4 h. The reaction mixture was purified by C18 column eluted with ACN/H₂O (v/v=2/8), filtered and concentrated under vacuum. This resulted in 1.351 g, (5.69 mmol, 55.09% yield) of (3-methoxy-4-(prop-2-yn-1-ylamino)phenyl)dimethylphosphine oxide as brown oil. LCMS: m/z=238 [M+1]⁺.

### Step 4 (4-((3-(7-((3,3-difluoro-1-methylpiperidin-4-yl)amino)-3-(2,2,2-trifluoroethyl)benzo[b]thiophen-2-yl)prop-2-yn-1-yl)amino)-3-methoxyphenyl)dimethylphosphine oxide (43)

Into a 10-mL round-bottom flask was placed (3-methoxy-4-(prop-2-yn-1-ylamino)phenyl)dimethylphosphine oxide (0.102 g, 429.95 μmol), 3,3-difluoro-N-(2-iodo-3-(2,2,2-trifluoroethyl)benzo[b]thiophen-7-yl)-1-methylpiperidin-4-amine (0.089 g, 181.53 μmol), Pd(PPh₃)₂Cl₂ (0.034 g, 48.16 μmol), CuI (0.022 g, 115.51 μmol), DI EA (0.105 g, 812.42 μmol), methyl sulfoxide (2 mL). The reaction was stirred under nitrogen atmosphere at RT for 16 h. The reaction was quenched with water (5 mL). The resulted solution was extracted with EA (3×10 mL), washed with brine (10 mL), dried over anhydrous sodium sulfate, filtered and concentrated under vacuum. The crude product was purified by preparative HPLC (Mobile Phase A: water (0.1% ammonium hydroxide), Mobile Phase B: ACN; Flow rate: 70 mL/min; Gradient: 55-85-100% B (2-32-50 min); 225 nm; RT: 30.53-31.56) to provide the desired product. This resulted in 0.010 g (16.67 μmol, 9.18% yield) of (4-((3-(7-((3,3-difluoro-1-methylpiperidin-4-yl)amino)-3-(2,2,2-trifluoroethyl)benzo[b]thiophen-2-yl)prop-2-yn-1-yl)amino)-3-methoxyphenyl)dimethylphosphine oxide (43) as white solid. LCMS: m/z=600 [M+1]⁺.

¹H NMR (400 MHz, DMSO-d₆) δ 7.28-7.12 (m, 4H), 6.85 (d, J=7.8 Hz, 2H), 6.06 (t, J=6.3 Hz, 1H), 5.38 (d, J=8.9 Hz, 1H), 4.34 (d, J=6.4 Hz, 2H), 3.85 (s, 3H), 3.79 (d, J=11.0 Hz, 2H), 2.78 (d, J=11.6 Hz, 2H), 2.25 (s, 3H), 2.17 (s, 1H), 1.95-1.82 (m, 3H), 1.58 (d, J=13.2 Hz, 6H), 1.24 (s, 1H).

### Example 44&45

### (R)-4-((4-(7-((1-methylpiperidin-4-yl)amino)-3-(2,2,2-trifluoroethyl)benzo[b]thiophen-2-yl)but-3-yn-2-yl)amino)benzenesulfonamide and (S)-4-((4-(7-((1-methylpiperidin-4-yl)amino)-3-(2,2,2-trifluoroethyl)benzo[b]thiophen-2-yl)but-3-yn-2-yl)amino)benzenesulfonamide (44&45)

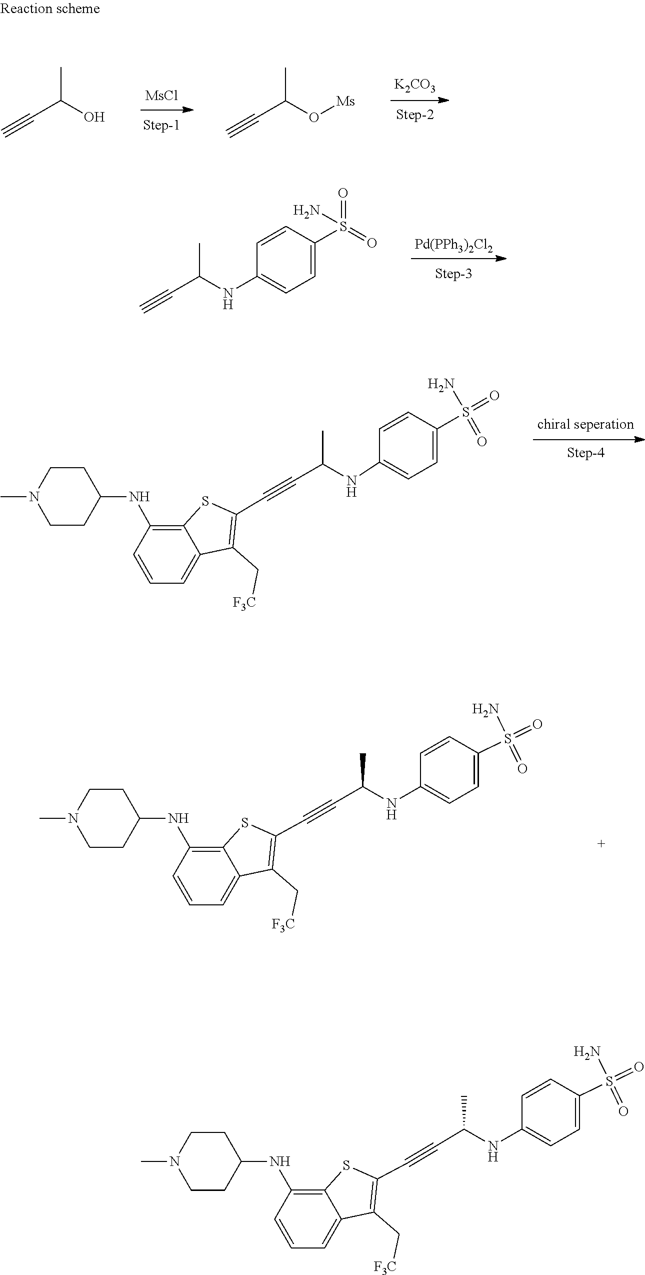

### Experimental Details

### Step 1. but-3-yn-2-yl methanesulfonate

Into a 100 mL flask was placed but-3-yn-2-ol (2.30 g, 32.82 mmol), DCM (20 mL), TEA (2 mL), MsCl (3 mL). The reaction was stirred at 20° C. for 4 h. The reaction was quenched by H₂O (20 mL), extracted by DCM (3×20 mL). The organic layers combined and concentrated under vacuum. This resulted in 1.99 g (crude) of but-3-yn-2-yl methanesulfonate as red oil. LCMS: m/z=149 [M+1]⁺.

### Step 2. 4-(but-3-yn-2-ylamino)benzenesulfonamide

Into a 100 mL flask was placed but-3-yn-2-yl methanesulfonate (1.83 g, 12.35 mmol), 4-aminobenzenesulfonamide (1.53 g, 9.06 mmol), Cs₂CO₃ (2.87 g, 8.81 mmol), DMF (3 mL). The reaction was stirred at 85° C. for 3 h. The reaction mixture was purified by C₁₈ chromatography column eluted with ACN/H₂O (0.15% TFA) (v/v=1/3). This resulted in 0.38 g (13% yield) of 4-(but-3-yn-2-ylamino)benzenesulfonamide as yellow solid. LCMS: m/z=225 [M+1]⁺.

### Step 3. 4-((4-(7-((1-methylpiperidin-4-yl)amino)-3-(2,2,2-trifluoroethyl)benzo[b]thiophen-2-yl)but-3-yn-2-yl) amino)benzenesulfonamide

Into a 100 mL flask was placed 4-(but-3-yn-2-ylamino)benzenesulfonamide (0.058 g, 258.61 μumol), N-(2-iodo-3-(2,2,2-trifluoroethyl)benzo[b]thiophen-7-yl)-1-methylpiperidin-4-amine (0.082 g, 180.50 μmol), CuI (0.047 g, 246.78 μmol), Pd(PPh₃)₂Cl₂ (0.081 g, 99.68 μmol), DIEA (0.050 g, 386.87 μmol) and Methyl sulfoxide (2 mL). The mixture was stirred at 25° C. for 4 h. The mixture was purified by preparative HPLC (Mobile Phase A: water (10 mmoL/L ammonium hydroxide), Mobile Phase B: ACN; Flow rate: 25 mL/min; Gradient: 50-70-90% B (2-30-60 min); 220 nm; RT: 33.580-36.110 min). This resulted in 53 mg (34% yield) of 4-((4-(7-((1-methylpiperidin-4-yl)amino)-3-(2,2,2-trifluoroethyl)benzo[b]thiophen-2-yl)but-3-yn-2-yl)amino)benzenesulfonamide(racemic) as white solid. LCMS: m/z=551 [M+1]⁺.

### Step 4. (R)-4-((4-(7-((1-methylpiperidin-4-yl)amino)-3-(2,2,2-trifluoroethyl)benzo[b]thiophen-2-yl)but-3-yn-2-yl)amino)benzenesulfonamide and (S)-4-((4-(7-((1-methylpiperidin-4-yl)amino)-3-(2,2,2-trifluoroethyl)benzo[b]thiophen-2-yl)but-3-yn-2-yl)amino)benzenesulfonamide (44&45)

4-((4-(7-((1-methylpiperidin-4-yl)amino)-3-(2,2,2-trifluoroethyl)benzo[b]thiophen-2-yl)but-3-yn-2-yl)amino)benzenesulfonamide(racemic) was separated via chiral separation to give 8 mg (18% yield) of (R)-4-((4-(7-((1-methylpiperidin-4-yl)amino)-3-(2,2,2-trifluoroethyl)benzo[b]thiophen-2-yl)but-3-yn-2-yl)amino)benzenesulfonamide as white solid. LCMS: m/z=551 [M+1]⁺.

¹H NMR (400 MHz, DMSO) δ 7.58 (d, J=8.6 Hz, 2H), 7.25 (t, J=7.8 Hz, 1H), 7.14 (d, J=7.9 Hz, 1H), 6.98 (s, 2H), 6.79 (t, J=7.8 Hz, 3H), 6.66 (d, J=7.8 Hz, 1H), 5.28 (d, J=8.1 Hz, 1H), 4.73-4.63 (m, 1H), 3.89-3.76 (m, 2H), 2.76 (d, J=11.5 Hz, 2H), 2.17 (s, 3H), 2.00 (t, J=11.4 Hz, 2H), 1.88 (d, J=12.3 Hz, 2H), 1.58 (d, J=6.7 Hz, 3H), 1.25 (d, J=9.4 Hz, 3H).

And 4-((4-(7-((1-methylpiperidin-4-yl)amino)-3-(2,2,2-trifluoroethyl)benzo[b]thiophen-2-yl)but-3-yn-2-yl)amino)benzenesulfonamide via chiral separation to give 10 mg (22% yield) of (S)-4-((4-(7-((1-methylpiperidin-4-yl)amino)-3-(2,2,2-trifluoroethyl)benzo[b]thiophen-2-yl)but-3-yn-2-yl)amino)benzenesulfonamide as white solid. LCMS: m/z=551 [M+1]⁺.

¹H NMR (400 MHz, DMSO) δ 7.58 (d, J=8.6 Hz, 2H), 7.25 (t, J=7.8 Hz, 1H), 7.14 (d, J=7.9 Hz, 1H), 6.98 (s, 2H), 6.79 (t, J=7.8 Hz, 3H), 6.66 (d, J=7.7 Hz, 1H), 5.29 (d, J=7.8 Hz, 1H), 4.66 (dd, J=14.0, 6.9 Hz, 1H), 3.92-3.74 (m, 2H), 2.77 (d, J=11.3 Hz, 2H), 2.18 (s, 3H), 2.01 (t, J=11.2 Hz, 2H), 1.88 (d, J=12.0 Hz, 2H), 1.41-1.25 (m, 3H), 1.23 (s, 3H).

### Example 46

### 4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-methoxybenzamide (racemic) (46)

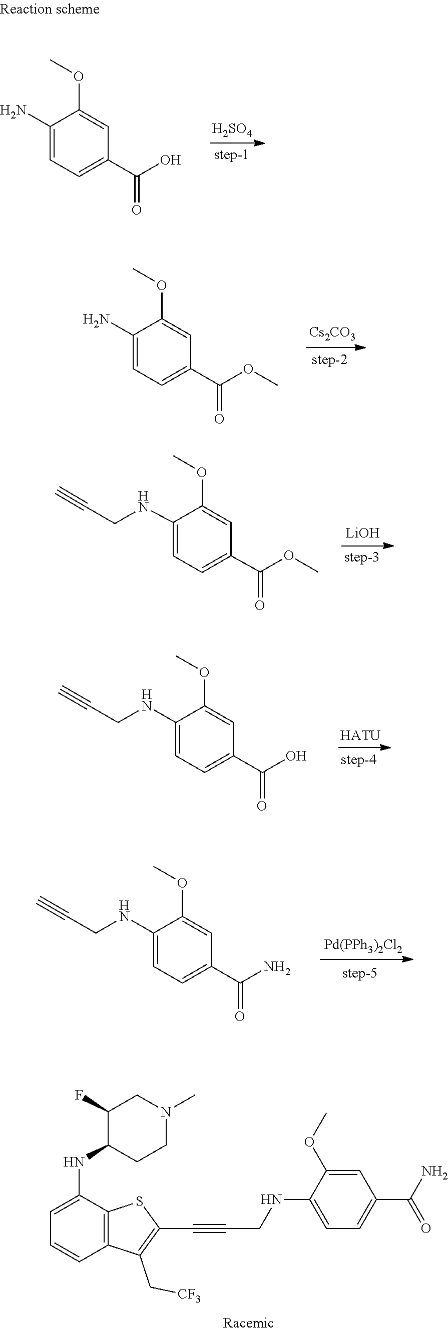

### Experimental Details

### Step 1. methyl 4-amino-3-methoxybenzoate

Into a 500 mL flask was placed 4-amino-3-methoxybenzoic acid (15.06 g, 90.09 mmol), MeOH (200 mL), sulfuric acid (15 mL). The reaction was stirred at 65° C. for 16 h. The reaction was quenched with NaOH (2N, 500 mL) at 10° C. Then an amount of precipitation appeared. The mixture was filtered and the filter cake was collected. The filter cake was dried at 60° C. for 16 h. This resulted in 14.35 g (87.91% yield) of methyl 4-amino-3-methoxybenzoate as white solid. LCMS: m/z=182 [M+1]⁺.

### Step 2. methyl 3-methoxy-4-(prop-2-yn-1-ylamino)benzoate

Into a 100 mL flask was placed methyl 4-amino-3-methoxybenzoate (4.15 g, 22.90 mmol), Cs₂CO₃ (3.73 g, 11.45 mmol), 3-bromoprop-1-yne (8.89 g, 74.73 mmol), KI (2.77 g, 16.69 mmol), DMF (20 mL). The reaction was stirred at 85° C. for 3 h. The reaction mixture was purified by C18 column eluted with ACN/H₂O (v/v=1/1). This resulted in 1.40 g (27% yield) of methyl 3-methoxy-4-(prop-2-yn-1-ylamino)benzoate as light yellow solid. LCMS: m/z=220 [M+1]⁺.

### Step 3. 3-methoxy-4-(prop-2-yn-1-ylamino)benzoic acid

Into a 25-mL round-bottom flask was placed methyl 3-methoxy-4-(prop-2-yn-1-ylamino)benzoate (0.82 g, 3.73 mmol), MeOH (4 mL), THF (4 mL), H₂O (4 mL), LiOH (0.81 g, 33.95 mmol). The reaction mixture was stirred at 50° C. for 3 h. The reaction was diluted with water (50 mL). Then Na₂CO₃ (aq, 100 mL) w as added until PH>8. Meanwhile a amount of precipitation appeared. The mixture was filtered and the filter cake was collected. The filter cake was dried at 60° C. for 16 h. This resulted in 0.75 g (97% yield) of 3-methoxy-4-(prop-2-yn-1-ylamino)benzoic acid as yellow solid. LCMS: m/z=206[M+1]⁺.

### Step 4. 3-methoxy-4-(prop-2-yn-1-ylamino)benzamide

Into a 50-mL round-bottom flask was placed 3-methoxy-4-(prop-2-yn-1-ylamino)benzoic acid (0.137 g, 667.61 μmol), NH₄Cl (0.324 g, 6.06 mmol), HATU (0.455 g, 1.20 mmol), DIEA (1.919 g, 14.85 mmol), DMF (5 mL). The mixture was stirred at 20° C. for 3 h. The reaction mixture was purified by C18 column elute d with ACN/H₂O (v/v=1/1). This resulted in 83 mg (60% yield) of 3-methoxy-4-(prop-2-yn-1-ylamino)benzamide as light yellow solid. LCMS: m/z=205 [M+1]⁺.

### Step 5. 4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-methoxybenzamide (racemic) (46)

Into a 50-mL round-bottom flask was placed 3-methoxy-4-(prop-2-yn-1-ylamino)benzamide (0.069 g, 337.86 μmol), (Z)-3-fluoro-N-(2-iodo-3-(2,2,2-trifluoroethyl)benzo[b]thiophen-7-yl)-1-methylpiperidin-4-amine (racemic) (0.106 g, 224.44 μmol), CuI (0.014 g, 73.51 μmol), Pd(PPh₃)₂Cl₂ (0.037 g, 52.41 μmol), DIEA (0.058 g, 448.77 μmol) and methyl sulfoxide (2 mL). The mixture was stirred at 25° C. for 4 h. The mixture was purified by preparative HPLC (Mobile Phase A: water (10 mmoL/L ammonium hydroxide), Mobile Phase B: ACN; Flow rate: 70 mL/min; Gradient: 50-80-100% B (2-32-60 min); 269 nm; RT: 33.448-35.501 mi n). This resulted in 43 mg (34% yield) of 4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-methoxybenzamide (racemic) (46) as white solid. LCMS: m/z=549 [M+1]⁺.

¹H NMR (400 MHz, DMSO) δ 7.69 (s, 1H), 7.47 (d, J=8.2 Hz, 1H), 7.39 (s, 1H), 7.30-7.17 (m, 2H), 6.98 (s, 1H), 6.81-6.69 (m, 2H), 6.05 (t, J=6.2 Hz, 1H), 5.17 (d, J=8.5 Hz, 1H), 4.79 (d, J=49.7 Hz, 1H), 4.34 (d, J=6.1 Hz, 2H), 3.84 (s, 3H), 3.80 (s, 1H), 3.64 (d, J=30.1 Hz, 2H), 3.02 (t, J=10.9 Hz, 1H), 2.79 (d, J=11.1 Hz, 1H), 2.32-2.21 (m, 1H), 2.17 (d, J=9.7 Hz, 3H), 2.07 (t, J=11.1 Hz, 1H), 2.01-1.86 (m, 1H), 1.71 (d, J=9.8 Hz, 1H).

### Example 47

### 4-((3-(7-((3-fluoro-1-methylpiperidin-4-yl)amino)-3-(2,2,2-trifluoroethyl)benzo[b]thiophen-2-yl)prop-2-yn-1-yl)amino)-3-methoxy-N-(1-methylpiperidin-4-yl)benzamide (racemic) (47)

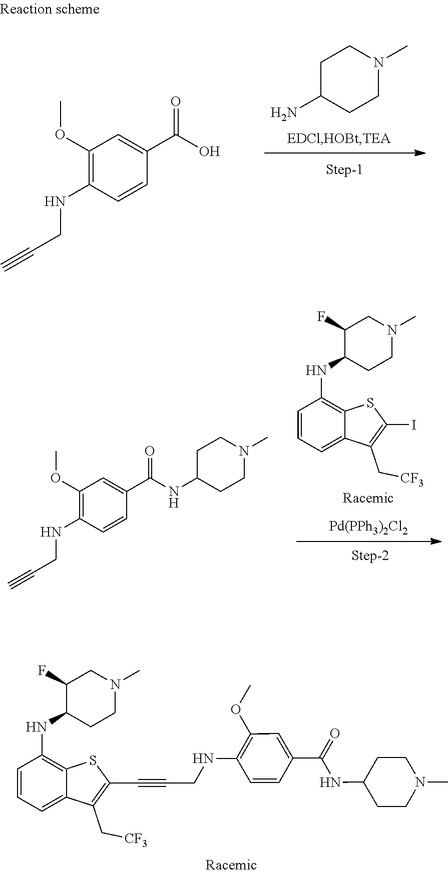

### Experimental Details

### Step 1. 3-methoxy-N-(1-methylpiperidin-4-yl)-4-(prop-2-yn-1-ylamino)benzamide

Into a 4 mL vail was placed 3-methoxy-4-(prop-2-yn-1-ylamino)benzoic acid (0.049 g, 238.78 μmol), 1-methylpiperidin-4-amine (0.044 g, 385.33 μmol), ECDI (0.049 g, 315.64 μmol), HOBT (0.043 g, 318.23 μmol), DMAP (0.003 g, 24.56 μmol), TEA (0.042 g, 415.06 μmol), DMF (0.5 mL). The reaction was stirred for 3 h at 40° C. LCMS showed the reaction was complete, the reaction purified with C₁₈ column, eluted with ACN/water (v/v=1/3) to afford 3-methoxy-N-(1-methylpiperidin-4-yl)-4-(prop-2-yn-1-ylamino)benzamide (0.050 g, 165.90 μmol, 69.48% yield) as clear oil. LCMS: m/z=302[M+1]⁺.

### Step 2. 4-((3-(7-((3-fluoro-1-methylpiperidin-4-yl)amino)-3-(2,2,2-trifluoroethyl)benzo[b]thiophen-2-yl)prop-2-yn-1-yl)amino)-3-methoxy-N-(1-methylpiperidin-4-yl)benzamide (racemic) (47)

Into a 4 mL flask purged and maintained with nitrogen atmosphere was placed 3-methoxy-N-(1-methylpiperidin-4-yl)-4-(prop-2-yn-1-ylamino)benzamide (0.049 g, 162.58 μmol), (Z)-3-fluoro-N-(2-iodo-3-(2,2,2-trifluoroethyl)benzo[b]thiophen-7-yl)-1-methylpiperidin-4-amine (racemic) (0.049 g, 103.75 μmol), Pd(PPh₃)₂Cl₂ (0.009 g, 12.75 μmol), CuI (0.003 g, 15.75 μmol), TEA (0.035 g, 345.89 μmol), DMF (0.5 mL), and stirred overnight at room temperature. LCMS showed the reaction was complete. The reaction was quenched with w ater (4 mL), extracted with EA (2 mL×2). The combined organic layers were washed with water (2 mL) and brine (2 mL) successively, separated, then concentrated with vacuum. The residue was purified with prep-HPLC (Mobile Phase A: water (ammonium hydroxide), Mobile Phase B: ACN; Flow rate: 50 mL/min; Gradient: 55-80-100% B (2-30-60 min); 248 nm; RT: 33.450-36.020 min) to afford 4-((3-(7-((3-fluoro-1-methylpiperidin-4-yl)amino)-3-(2,2,2-trifluoroethyl)benzo[b]thiophen-2-yl)prop-2-yn-1-yl)amino)-3-methoxy-N-(1-methylpiperidin-4-yl)benzamide (racemic) (47) (42 mg, 65.04 μmol, 40.00% yield) as an off-white solid. LCMS: m/z=646[M+1]⁺

¹H NMR (400 MHz, DMSO-d₆) δ 7.88 (d, J=7.8 Hz, 1H), 7.46 (d, J=8.2 Hz, 1H), 7.36 (s, 1H), 7.32-7.16 (m, 2H), 6.77 (dd, J=10.9, 7.9 Hz, 2H), 6.02 (t, J=6.5 Hz, 1H), 5.15 (d, J=8.5 Hz, 1H), 4.79 (d, J=49.0 Hz, 1H), 4.34 (d, J=6.3 Hz, 2H), 3.95-3.77 (m, 5H), 3.76-3.55 (m, 2H), 3.03 (t, J=11.3 Hz, 1H), 2.77 (d, J=11.3 Hz, 3H), 2.27 (d, J=13.2 Hz, 1H), 2.17 (d, J=9.2 Hz, 6H), 2.10-1.88 (m, 4H), 1.80-1.66 (m, 3H), 1.63-1.52 (m, 2H).

### Example 48

### (1,1-dioxidothiomorpholino)(4-((3-(7-(((3R,4S)-3-fluoro-1-methylpiperidin-4-yl)amino)-3-(2,2,2-trifluoroethyl) benzo[b]thiophen-2-yl)prop-2-yn-1-yl)amino)-3-methoxyphenyl)methanone (racemic) (48)

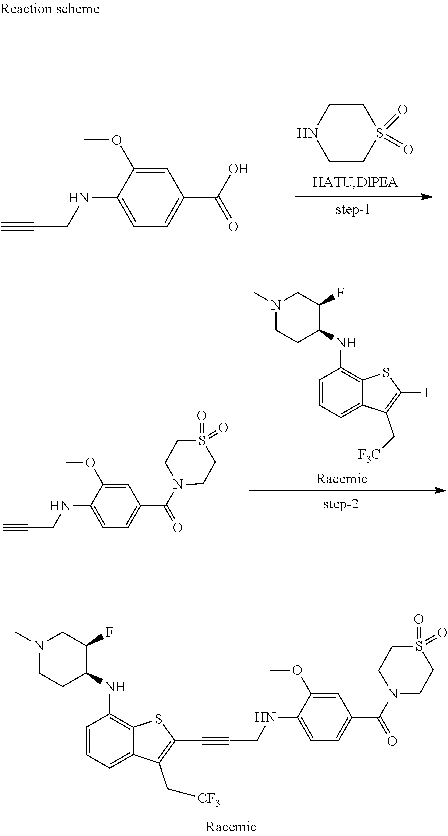 [SMILES: C#CCNc1ccc(C(=O)O)cc1OC ; O=S1(=O)CCNCC1 ; C#CCNc1ccc(C(=O)N2CCS(=O)(=O)CC2)cc1OC ; CN1CC[C@H](Nc2cccc3c(CC(F)(F)F)c(I)sc23)[C@H](F)C1 ; COc1cc(C(=O)N2CCS(=O)(=O)CC2)ccc1NCC#Cc1sc2c(N[C@H]3CCN(C)C[C@H]3F)cccc2c1CC(F)(F)F]

### Experimental Details

### Step 1. (1,1-dioxidothiomorpholino)(3-methoxy-4-(prop-2-yn-1-ylamino)phenyl)methanone

Into a 8 mL flask purged and maintained with an inert atmosphere of nitrogen, was placed 3-methoxy-4-(prop-2-yn-1-ylamino)benzoic acid (0.053 g, 258.27 μmol), 4-thiomorpholine-1,1-dione hydrochloride (0.139 g, 809.81 μmol), 2-(7-aza-1H-benzotriazole-1-yl)-1,1,3,3-tetramethyluronium hexafluorophosphate (0.366 g, 962.58 μmol), N,N-diisopropylethylamine (0.119 g, 920.75 μmol), N,N-dimethylformamide (2 mL). The reaction mixture was stirred at room temperature for 18 h. The reaction was quenched by the addition of water (2 mL) and extracted with EA (2×2 mL). The organic layers was combined, washed with 15% potassium carbonate solution (2×2 mL) and brine (5 mL), dried over anhydrous Na₂SO₄ and concentrated under vacuum. The residues were purified by C₁₈ chromatography column eluted with ACN/H₂O (v/v=3/1). This resulted in 0.092 g (110.50% yield) of (1,1-dioxidothiomorpholino)(3-methoxy-4-(prop-2-yn-1-ylamino) phenyl)methanone (crude) as white solid. LCMS: m/z=323 [M+1]⁺.

### Step 2. (1,1-dioxidothiomorpholino)(4-((3-(7-(((3R,4S)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)methanone (racemic) (48)

Into a 100 mL flask purged and maintained with an inert atmosphere of nitrogen, was placed (3R,4S)-3-fluoro-N-(2-iodo-3-(2,2,2-trifluoroethyl)benzo[b]thiophen-7-yl)-1-methylpiperidin-4-amine (racemic) (0.103 g, 218.09 μmol), cuprous iodide (0.008 g, 42.01 μmol), (1,1-dioxidothiomorpholino)(3-methoxy-4-(prop-2-yn-1-ylamino)phenyl)methanone (0.084 g, 260.5629 μmol), bis(triphenylphosphine)palladium(II) chloride (0.021 g, 29. 75 μmol), triethylamine (0.153 g, 1.51 mmol), methyl sulfoxide (1 mL). The reaction mixture was stirred a t room temperature for 5 h. The reaction was quenched by the addition of water (2 mL) and extracted with EA (2×2 mL). The organic layers was combined, washed with brine (5 mL), dried over anhydrous Na₂SO₄ and concentrated under vacuum. The mixture was purified by preparative HPLC (Mobile Phase A: w ater (10 mmoL/L ammonium hydroxide), Mobile Phase B: MeOH; Flow rate: 70 mL/min; Gradient: 55-80-100% B (2-32-60 min); 269 nm; RT: 30.797-31.863 min). This resulted in 0.026 g (17.88% yield) of (1,1-dioxidothiomorpholino)(4-((3-(7-(((3R,4S)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)methanone (racemic) (48) as white solid. LCMS: m/z=667 [M+1]⁺.

¹H NMR (400 MHz, DMSO) δ 7.31-7.19 (m, 2H), 7.02 (dd, J=10.5, 2.5 Hz, 2H), 6.82-6.74 (m, 2H), 6.02 (t, J=6.3 Hz, 1H), 5.16 (d, J=8.6 Hz, 1H), 4.80 (d, J=49.6 Hz, 1H), 4.34 (d, J=6.3 Hz, 2H), 3.90 (s, 4H), 3.86-3.76 (m, 5H), 3.74-3.55 (m, 1H), 3.25 (d, J=4.4 Hz, 4H), 3.03 (t, J=10.8 Hz, 1H), 2.80 (d, J=12.0 Hz, 1H), 2.25 (t, J=13.1 Hz, 1H), 2.18 (s, 3H), 2.08 (t, J=11.1 Hz, 1H), 2.02-1.89 (m, 1H), 1.72 (d, J=9.8 Hz, 1H).

### Example 49

### (4-((3-(7-(((Z)-3-fluoro-1-methylpiperidin-4-yl)amino)-3-(2,2,2-trifluoroethyl)benzo[b]thiophen-2-yl)prop-2-yn-1-amino)-3-methoxyphenyl)(4-methylpiperazin-1-yl)methanone (racemic) (49)

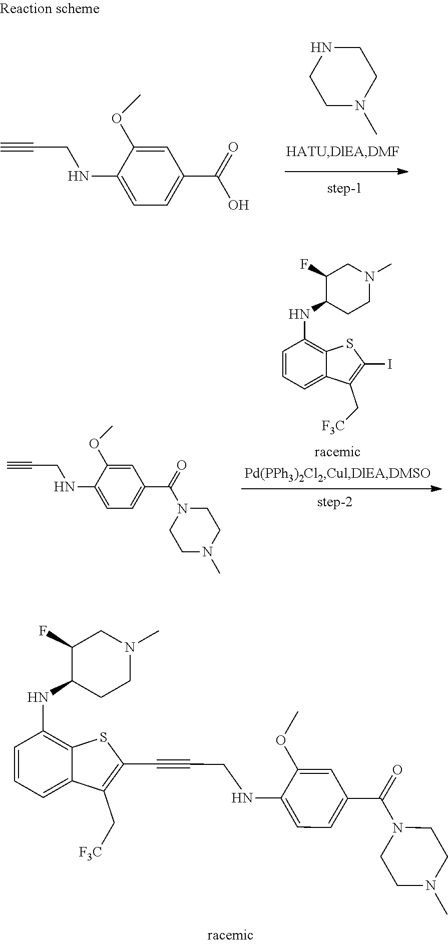

### Experimental Details

### Step 1. (3-methoxy-4-(prop-2-yn-1-ylamino)phenyl)(4-methylpiperazin-1-yl)methanone

Into a 4 mL sealed tube was placed 3-methoxy-4-(prop-2-yn-1-ylamino)benzoic acid (0.101 g, 492.18 μmol), 1-methylpiperazine (0.107 g, 1.07 mmol), 2-(7-aza-1H-benzotriazole-1-yl)-1,1,3,3-tetramethyluronium hexafluorophosphate (0.324 g, 852.12 μmol), N,N-diisopropylethylamine (0.205 g, 1.59 mmol), N,N-dimethylformamide (2 mL). The reaction was stirred overnight at RT. The reaction was quenched with water (50 mL), extracted with EA (20 mL×2). The combined organic layers was washed with water (20 mL) and brine (30 mL×2) successively, separated and concentrated under vacuum. The crude was purified by silica gel column eluted with MeOH/DCM (v/v=1/9). This resulted in 0.19 g (crude) of (3-methoxy-4-(prop-2-yn-1-ylamino)phenyl)(4-methylpiperazin-1-yl)methanone as brown-yellow oil. LCMS: m/z=288 [M+1]⁺.

### Step 2. (4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)(4-methylpiperazin-1-yl)methanone (racemic) (49)

Into a 25-mL round-bottom flask was placed (3-methoxy-4-(prop-2-yn-1-ylamino)phenyl)(4-methylpiperazin-1-yl)methanone (0.167 g, 581.16 μmol), (Z)-3-fluoro-N-(2-iodo-3-(2,2,2-trifluoroethyl)benzo[b]thiophen-7-yl)-1-m ethylpiperidin-4-amine (racemic) (0.100 g, 211.74 μmol), bis(triphenylphosphine)palladium(II) chloride (0.043 g, 60.91 μmol), CuI (0.036 g, 189.03 mmol), DIEA (0.164 g, 1.27 mmol), methyl sulfoxide (5 mL). The reaction was stirred at 60° C. for 2 h under nitrogen atmosphere. The reaction was quenched with water (20 mL), extracted with EA (20 mL×2). The combined organic layers was washed with water (30 mL) and brine (30 mL) successively, separated and concentrated under vacuum. The mixture was purified by preparative HPLC (Mobile Phase A: water (10 mmoL/L ammonium hydroxide), Mobile Phase B: MeOH; Flow rate: 70 mL/min; Gradient: 45-80-100% B (2-32-60 min); 269 nm; RT: 39.863-41.317 min) to provide the desired product. This resulted in 0.061 g (45.60% yield) of (4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)(4-methylpiperazin-1-yl)methanone (racemic) (49) as off-white solid. LCMS: m/z=632 [M+1]⁺.

¹H NMR (400 MHz, MeOD) δ 7.29 (t, J=7.8 Hz, 1H), 7.19 (d, J=7.8 Hz, 1H), 7.01 (d, J=8.1 Hz, 1H), 6.96 (s, 1H), 6.85 (d, J=8.2 Hz, 1H), 6.77 (d, J=7.7 Hz, 1H), 4.96-4.89 (m, 1.5H), 4.83-4.73 (m, 0.5H), 4.35 (s, 2H), 3.89 (s, 3H), 3.77-3.62 (m, 6H), 3.25-3.15 (m, 1H), 2.98-2.88 (m, 1H), 2.56-2.42 (m, 4H), 2.42-2.36 (m, 1H), 2.30 (d, J=7.1 Hz, 6H), 2.27-2.20 (m, 1H), 2.01-1.91 (m, 2H).

### Example 50

### (4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)(morpholino)methanone (racemic) (50)

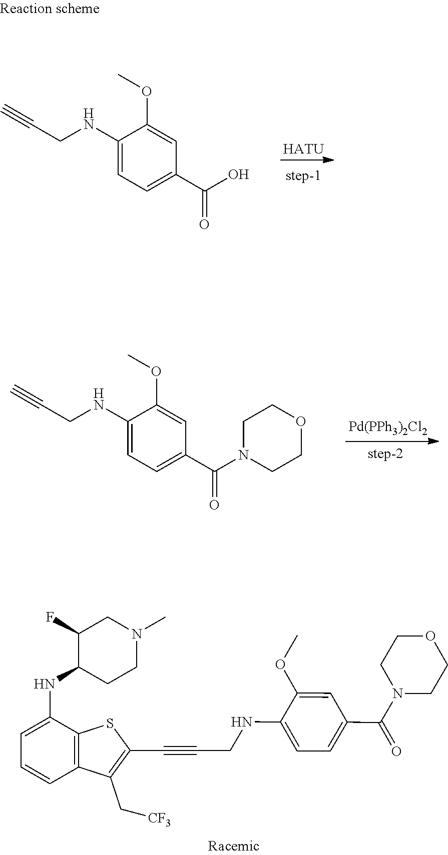

### Experimental Details

### Step 1. (3-methoxy-4-(prop-2-yn-1-ylamino)phenyl)(morpholino)methanone

Into a 50-mL round-bottom flask was placed 3-methoxy-4-(prop-2-yn-1-ylamino)benzoic acid (0.065 g, 316.74 μmol), morpholine (0.125 g, 1.43 mmol), HATU (0.280 g, 0.74 mmol), DIEA (0.296 g, 2.29 mmol), D MF (5 mL). The mixture was stirred at 20° C. for 3 h. The reaction mixture was purified by C18 column eluted with ACN/H₂O (v/v=1/1). This resulted in 94 mg (98% yield) of (3-methoxy-4-(prop-2-yn-1-ylamino) phenyl)(morpholino)methanone as light yellow solid. LCMS: m/z=275 [M+1]⁺.

### Step 2. (4-((3-(7-(((Z)-3-fluoro-1-methylpiperidin-4-yl)amino)-3-(2,2,2-trifluoroethyl)benzo[b]thiophen-2-yl) prop-2-yn-1-yl)amino)-3-methoxyphenyl)(morpholino)methanone (racemic) (50)

Into a 50-mL round-bottom flask was placed (3-methoxy-4-(prop-2-yn-1-ylamino)phenyl)(morpholino)methano ne (0.069 g, 251.54 μmol), (Z)-3-fluoro-N-(2-iodo-3-(2,2,2-trifluoroethyl)benzo[b]thiophen-7-yl)-1-methylpiperidin-4-amine (0.106 g, 224.44 μmol), CuI (0.014 g, 73.51 μmol), Pd(PPh₃)₂Cl₂ (0.037 g, 52.41 μmol), DIE A (0.058 g, 448.77 μmol) and methyl sulfoxide (2 mL). The mixture was stirred at 25° C. for 4 h. The mixture was purified by preparative HPLC (Mobile Phase A: water (10 mmoL/L ammonium hydroxide), Mobile Phase B: ACN; Flow rate: 70 mL/min; Gradient: 55-80-100% B (2-32-60 min); 269 nm; RT: 37.007-38.908 min). This resulted in 41 mg (29% yield) of (4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)(morpholino)methanone (racemic) (50) as white solid. LCMS: m/z=619 [M+1]⁺.

¹H NMR (400 MHz, DMSO) δ 7.34-7.15 (m, 2H), 7.02-6.88 (m, 2H), 6.77 (m, 2H), 5.98 (t, J=6.3 Hz, 1H), 5.16 (d, J=8.6 Hz, 1H), 4.79 (d, J=49.3 Hz, 1H), 4.33 (d, J=6.3 Hz, 2H), 3.86-3.82 (mii, 3H), 3.82-3.75 (m, 2H), 3.73-3.65 (m, 1H), 3.59 (d, J=3.9 Hz, 4H), 3.52 (d, J=3.8 Hz, 4H), 3.03 (t, J=10.7 Hz, 1H), 2.79 (d, J=11.0 Hz, 1H), 2.36-2.21 (m, 1H), 2.17 (d, J=10.6 Hz, 3H), 2.07 (t, J=11.2 Hz, 1H), 2.01-1.88 (m, 1H), 1.71 (d, J=9.7 Hz, 1H)

### Example 51

### N-(4-fluoro-2-(3-((2-methoxy-4-(methylsulfonyl)phenyl)amino)prop-1-yn-1-yl)-3-(2,2,2-trifluoroethyl)benzo[b]thiophen-7-yl)-1-methylpiperidin-4-amine (51)

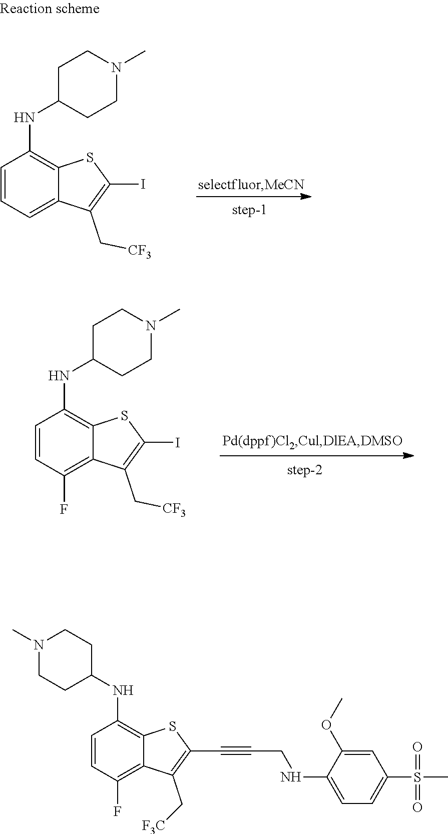

### Experimental Details

### Step 1. N-(4-fluoro-2-iodo-3-(2,2,2-trifluoroethyl)benzo[b]thiophen-7-yl)-1-methylpiperidin-4-amine

Into a 20-mL sealed tube purged and maintained with an inert atmosphere of nitrogen, was placed N-(2-iodo-3-(2,2,2-trifluoroethyl)benzo[b]thiophen-7-yl)-1-methylpiperidin-4-amine (0.408 g, 0.90 mmol), selectfluor (0.375 g, 1.06 mmol), MeCN (10 mL). The reaction mixture was stirred at room temperature for 1 h. The mixture purified by silica gel column, eluted with EA/hexane (v/v=1/4) to afford 0.304 g (71.67%) of N-(4-fluoro-2-iodo-3-(2,2,2-trifluoroethyl)benzo[b]thiophen-7-yl)-1-methylpiperidin-4-amine as yellow oil. LCMS: m/z=473 [M+1]⁺

### Step 2. N-(4-fluoro-2-(3-((2-methoxy-4-(methylsulfonyl)phenyl)amino)prop-1-yn-1-yl)-3-(2,2,2-trifluoroethyl) benzo[b]thiophen-7-yl)-1-methylpiperidin-4-amine (51)

Into a 20-mL sealed tube purged and maintained with an inert atmosphere of nitrogen, was placed N-(4-fluoro-2-iodo-3-(2,2,2-trifluoroethyl)benzo[b]thiophen-7-yl)-1-methylpiperidin-4-amine (0.268 g, 0.57 mmol), 2-m ethoxy-4-(methylsulfonyl)-N-(prop-2-yn-1-yl)aniline (0.271 g, 1.13 mmol), Pd(dppf)Cl₂ (0.098 g, 0.14 mmol), CuI (0.164 g, 0.86 mmol), DIEA (0.314 g, 2.43 mmol), DMSO (5 mL). The reaction mixture was stirred at 50° C. for 1 h. The reaction mixture was quenched by the addition of water (20 mL), extracted with E A (20 mL×2). The combined organic layers was washed with brine (10 mL), separated and concentrated under vacuum. The mixture was purified by preparative HPLC (Mobile Phase A: water (10 mmoL/L ammonium hydroxide), Mobile Phase B: MeCN; Flow rate: 60 mL/min; Gradient: 50-75-95% B (2-30-55 min); 242 nm; RT: 34.125-36.751 min) to afford N-(4-fluoro-2-(3-((2-methoxy-4-(methylsulfonyl)phenyl)amino)prop-1-yn-1-yl)-3-(2,2,2-trifluoroethyl)benzo[b]thiophen-7-yl)-1-methylpiperidin-4-amine (51) (0.092 g, 27.78% yield) as white solid. LCMS: m/z=584 [M+1]⁺

¹H NMR (400 MHz, MeOD) δ 7.52-7.46 (m, 1H), 7.32-7.23 (m, 2H), 7.23-7.13 (m, 1H), 6.93 (d, J=8.4 Hz, 1H), 4.40 (s, 2H), 3.95 (s, 3H), 3.74-3.63 (m, 2H), 3.51 (d, J=22.0 Hz, 1H), 3.06 (s, 3H), 2.86 (d, J=11.8 Hz, 2H), 2.29 (s, 3H), 2.14 (t, J=11.2 Hz, 2H), 1.92 (d, J=12.4 Hz, 2H), 1.67-1.51 (m, 2H).

### Example 52

(2-fluoro-4-((3-(7-(((Z)-3-fluoro-1-methylpiperidin-4-yl)amino)-3-(2,2,2-trifluoroethyl)benzo[b]thiophen-2-yl) prop-2-yn-1-yl)amino)-5-methoxyphenyl)dimethylphosphine oxide (racemic) (52)

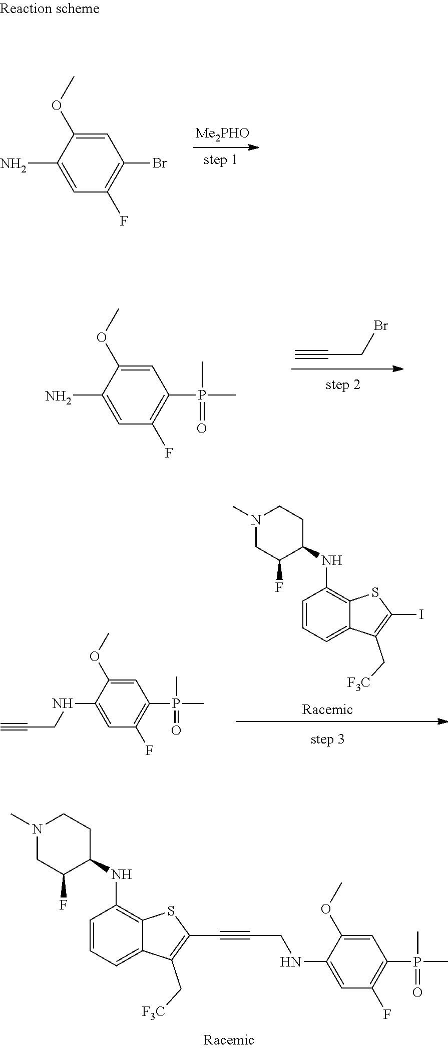

### Experimental Details

### Step 1. Synthesis of (4-amino-2-fluoro-5-methoxyphenyl)dimethylphosphine oxide

Into a 40-mL vial was placed 4-bromo-5-fluoro-2-methoxyaniline (1.084 g, 4.93 mmol), dimethylphosphine oxide (0.508 g, 6.51 mmol), palladium (II) acetate (0.150 g, 668.13 μmol), dimethylbisdiphenylphosphinoxanthene (0.652 g, 1.13 mmol) N,N-Diisopropylethylamine (1.495 g, 11.57 mmol), DMF (10 mL). The mixture was stirred at 130° C. under nitrogen atmosphere overnight. Then the resulted reaction was purified by C₁₈ chromatography column eluted with ACN/water (v/v=1/6). This resulted in 0.955 g (89.26% yield) of (4-amino-2-fluoro-5-methoxyphenyl)dimethylphosphine oxide. LCMS: m/z=218 [M+1]⁺.

### Step 2. Synthesis of (2-fluoro-5-methoxy-4-(prop-2-yn-1-ylamino)phenyl)dimethylphosphine oxide

Into a 40-mL vial was placed (4-amino-2-fluoro-5-methoxyphenyl)dimethylphosphine oxide (0.654 g, 3.01 m mol), DMA (15 mL), cesium carbonate (3.149 g, 9.67 mmol), sodium iodide (0.413 g, 2.76 mmol), N-(4-pyridyl)dimethylamine (0.083 g, 679.40 μmol), 3-bromoprop-1-yne (0.746 g, 6.27 mmol). Then the mixture was stirred at 90° C. overnight. The resulted reaction was purified by C₁₈ chromatography column eluted with ACN/water (v/v=1/3). This resulted in 0.255 g (33.18% yield) of (2-fluoro-5-methoxy-4-(prop-2-yn-1-yl amino)phenyl)dimethylphosphine oxide. LCMS: m/z=256 [M+1]⁺.

### Step 3. Synthesis of (2-fluoro-4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-5-methoxyphenyl)dimethylphosphine oxide (racemic) (52)

Into a 40-mL vial was placed (2-fluoro-5-methoxy-4-(prop-2-yn-1-ylamino)phenyl)dimethylphosphine oxide (0.251 g, 983.45 μmol), (Z)-3-fluoro-N-(2-iodo-3-(2,2,2-trifluoroethyl)benzo[b]thiophen-7-yl)-1-methylpiperidin-4-amine (racemic) (0.470 g, 995.17 μmol), bis(triphenylphosphine)palladium(II) chloride (0.070 g, 99.16 μmol), copper(I) iodide (0.034 g, 178.52 μmol) N,N-Diisopropylethylamine (0.437 g, 3.38 mmol), methyl sulfoxide (10 mL). The mixture was stirred at 40° C. for 4 h. The resulted reaction was diluted with water (30 mL) and extracted with EA (10 mL×3). The organic phase was dried over Na₂SO₄ and concentrated under vacuum. The resulted reaction was purified by C₁₈ chromatography column eluted with ACN/water (0.1% ammonium bicarbonate) (v/v=1/1) to give the crude product. Then the crude was concentrated and purified by C₁₈ chromatography column eluted with ACN/water (0.1% formic acid) (v/v=1/1) to give the product. This resulted in 0.100 g (16.96% yield) of (2-fluoro-4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-5-methoxyphenyl)dimethylphosphine oxide (racemic) (52) (HCOOH salt) as white solid. LCMS: m/z=600 [M+1]⁺.

¹H NMR (400 MHz, DMSO-d₆) δ 8.15 (s, 1H), 7.27 (t, J=7.8 Hz, 1H), 7.21 (d, J=7.9 Hz, 1H), 7.02 (dd, J=12.3, 5.8 Hz, 1H), 6.78 (d, J=7.7 Hz, 1H), 6.65 (dd, J=11.9, 5.0 Hz, 1H), 6.45 (t, J=6.4 Hz, 1H), 5.18 (d, J=8.5 Hz, 1H), 4.74 (s, 1H), 4.35 (d, J=6.2 Hz, 2H), 3.82 (s, 3H), 3.79 (d, J=11.0 Hz, 1H), 3.69 (s, 1H), 3.62 (s, 1H), 3.04 (t, J=11.1 Hz, 1H), 2.80 (d, J=11.0 Hz, 1H), 2.33-2.67 (m, 1H), 2.19 (s, 3H), 2.12-2.06 (m, 1H), 1.99-1.90 (m, 1H), 1.76-1.68 (m, 1H), 1.62 (d, J=13.5 Hz, 6H).

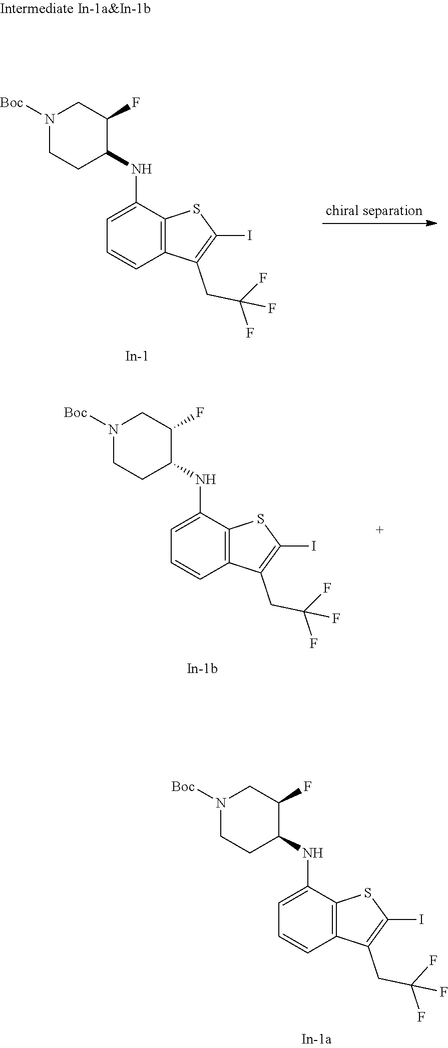

### Experiment Details

### tert-butyl (3R,4S)-3-fluoro-4-((2-iodo-3-(2,2,2-trifluoroethyl)benzo[b]thiophen-7-yl)amino)piperidine-1-carboxylate (In-1a) & tert-butyl (3S,4R)-3-fluoro-4-((2-iodo-3-(2,2,2-trifluoroethyl)benzo[b]thiophen-7-yl)amino)piperidine-1-carboxylate (In-1b)

The racemic product In-1 (7.987 g, 14.30 mmol) was separated by Prep-HPLC-Gilson with the following conditions: Column, CHIRALART Cellulose-IG column (2 cm×25 cm, 5 um); mobile phase, Hexane:EtOH (V/V=75:25); Flowing rate: 18 ml/min. This results in (2.48 g, 4.44 mmol) tert-butyl (3R,4S)-3-fluoro-4-((2-iodo-3-(2,2,2-trifluoroethyl)benzo[b]thiophen-7-yl)amino)piperidine-1-carboxylate (In-1a) (the first eluting isomer, Retention Time 5.804 min). LCMS: m/z=559 [M+1]⁺

¹H NMR (400 MHz, DMSO-d₆) δ 7.27-7.18 (m, 2H), 6.76-6.71 (m, 1H), 5.33 (d, J=8.6 Hz, 1H), 4.83 (d, J=48.8 Hz, 1H), 4.24 (s, 1H), 4.13-3.98 (m, 1H), 3.92-3.78 (m, 3H), 3.25-2.75 (m, 2H), 1.92-1.68 (m, 2H), 1.41 (s, 9H).

And (2.48 g, 4.44 mmol) tert-butyl (3S,4R)-3-fluoro-4-((2-iodo-3-(2,2,2-trifluoroethyl)benzo[b]thiophen-7-yl)amino)piperidine-1-carboxylate (In-1b) (the second eluting isomer, Retention Time 6.645 min) LCMS: m/z=559 [M+1]⁺

¹H NMR (400 MHz, DMSO-d₆) δ 7.27-7.18 (m, 2H), 6.76-6.71 (m, 1H), 5.33 (d, J=8.6 Hz, 1H), 4.83 (d, J=48.8 Hz, 1H), 4.24 (s, 1H), 4.14-3.98 (m, 1H), 3.92-3.78 (m, 3H), 3.24-2.76 (m, 2H), 1.91-1.68 (m, 2H), 1.41 (s, 9H).

Intermediate In-2a & In-2b

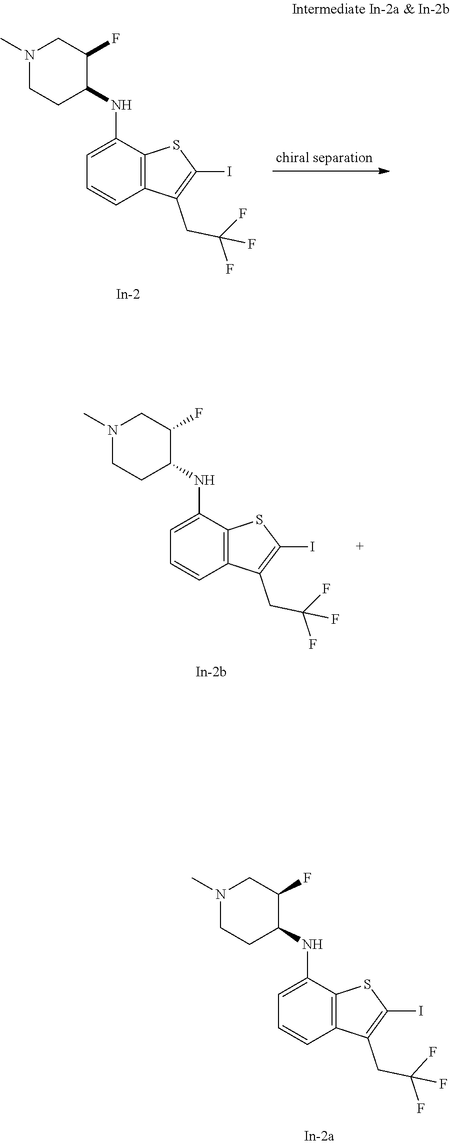

### Experiment Details

### (3R,4S)-3-fluoro-N-(2-iodo-3-(2,2,2-trifluoroethyl)benzo[b]thiophen-7-yl)-1-methylpiperidin-4-amine (In-2a) & (3S,4R)-3-fluoro-N-(2-iodo-3-(2,2,2-trifluoroethyl)benzo[b]thiophen-7-yl)-1-methylpiperidin-4-amine (In-2b)

The racemic product In-2 (7.987 g, 14.30 mmol) was separated by Prep-HPLC-Gilson with the following conditions: Column, CHIRALART Cellulose-IG column (2 cm×25 cm, 5 um); mobile phase, Hexane:EtOH (V/V=80:20); Flowing rate: 18 ml/min. This results in (2.48 g, 4.44 mmol) (3R,4S)-3-fluoro-N-(2-iodo-3-(2,2,2-trifluoroethyl)benzo[b]thiophen-7-yl)-1-methylpiperidin-4-amine (In-2a) (the first eluting isomer, Retention Time 6.175 min). LCMS: m/z=473 [M+1]⁺

¹H NMR (400 MHz, DMSO-d₆) δ 7.30-7.16 (m, 2H), 6.71 (dd, J=6.2, 2.6 Hz, 1H), 5.18 (d, J=8.6 Hz, 1H), 4.91-4.66 (m, 1H), 3.84 (q, J=11.1 Hz, 2H), 3.74-3.57 (m, 1H), 3.09-3.01 (m, 1H), 2.85-2.75 (m, 1H), 2.19 (s, 4H), 2.13-2.03 (m, 1H), 2.00-1.90 (m, 1H), 1.80-1.66 (m, 1H).

And (2.48 g, 4.44 mmol) (3S,4R)-3-fluoro-N-(2-iodo-3-(2,2,2-trifluoroethyl)benzo[b]thiophen-7-yl)-1-methylpiperidin-4-amine (In-2b) (the second eluting isomer, Retention Time 7.982 min) LCMS: m/z=473 [M+1]⁺

¹H NMR (400 MHz, DMSO-d₆) δ 7.30-7.16 (m, 2H), 6.71 (dd, J=6.2, 2.6 Hz, 1H), 5.18 (d, J=8.6 Hz, 1H), 4.91-4.66 (m, 1H), 3.84 (q, J=11.1 Hz, 2H), 3.74-3.57 (m, 1H), 3.09-3.01 (m, 1H), 2.85-2.75 (m, 1H), 2.19 (s, 4H), 2.13-2.03 (m, 1H), 2.00-1.90 (m, 1H), 1.80-1.66 (m, 1H).

### Example 53

### 2-(3-((2-methoxy-4-(methylsulfonyl)phenyl)amino)prop-1-yn-1-yl)-N-(1-methylpiperidin-4-yl)-3-(2,2,2-trifluoroethyl)thieno[3,2-b]pyridin-7-amine (53)

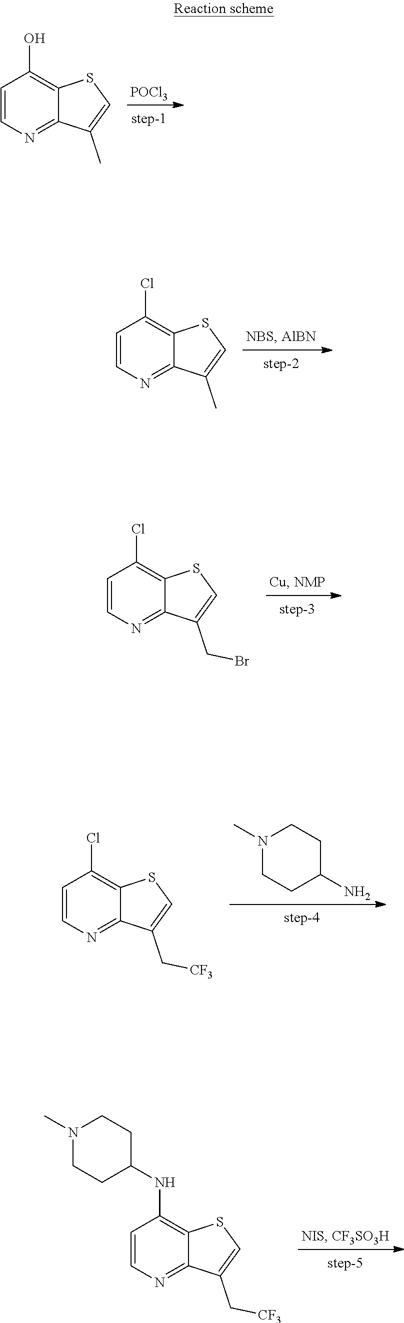

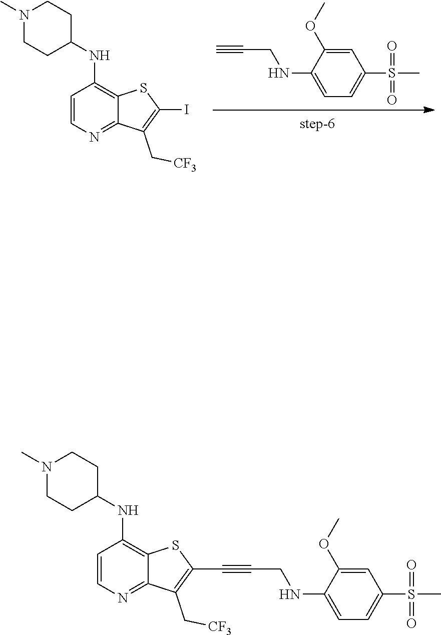

### Experimental Details

### Step 1. 7-chloro-3-methylthieno[3,2-b]pyridine

Into a 100-mL three necked bottle purged and maintained with an inert atmosphere of nitrogen, was placed 3-methylthieno[3,2-b]pyridin-7-ol (1.00 g, 6.07 mmol), POCl₃ (6 mL). The reaction mixture was stirred at 110° C. for 1.5 h. The reaction mixture was cooled to room temperature and POCl₃ was concentrated under vacuum. The resulting solution was added to water (50 mL). The resulting solution was extracted with E A (2×50 mL). The organic layers combined and washed with brine (20 mL), dried over anhydrous Na₂S O₄, the residue was concentrated under vacuum. The residue was applied onto a silica gel column eluted with EA:hexane (v:v=1/3). This resulted in 1.10 g (98.55% yield) of 7-chloro-3-methylthieno[3,2-b]pyridine as yellow solid. LCMS: m/z=183 [M+1]⁺

### Step 2. 3-(bromomethyl)-7-chlorothieno[3,2-b]pyridine

Into a 100-mL three necked bottle purged and maintained with an inert atmosphere of nitrogen, was placed 7-chloro-3-methylthieno[3,2-b]pyridine (1.80 g, 9.80 mmol), NBS (2.50 g, 14.04 mmol), AIBN (200.00 mg, 1.21 mmol), EA (20 mL). The mixture was stirred at 80° C. for 5 h. The resulting solution was cooled to room temperature and quenched with water (100 mL). The resulting solution was extracted with EA (2×100 mL). The organic layers combined and washed with brine, dried over anhydrous Na₂SO₄, the residue was concentrated under vacuum. The residue was applied onto a silica gel column eluted with EA:hexane (v:v=1:10). This resulted in 1.50 g (58.29% yield) of 3-(bromomethyl)-7-chlorothieno[3,2-b]pyridine as yellow solid. LCMS: m/z=262 [M+1]⁺.

### Step 3. 7-chloro-3-(2,2,2-trifluoroethyl)thieno[3,2-b]pyridine

Into a 100-mL three necked bottle purged and maintained with an inert atmosphere of nitrogen, was placed 3-(bromomethyl)-7-chlorothieno[3,2-b]pyridine (1.01 g, 3.84 mmol), diphenyl-(trifluoromethyl)-sulfonium trifluoromethanesulfonate (3.62 g, 8.95 mmol), Cu (0.71 g, 11.17 mmol), NMP (20 mL). The reaction mixture w as stirred at 60° C. for 4 h. The resulting solution was cooled to room temperature and quenched with H₂O (100 mL). The resulting solution was filtered through a celite pad, and the filtrate extracted with EA (2×100 mL). The organic layers combined and washed with brine (50 mL), dried over anhydrous Na₂SO₄, the residue was concentrated under vacuum. The residue was applied onto a silica gel column eluted with EA:hexane (v:v=1:10). This resulted in 800.00 mg (82.63% yield) of 7-chloro-3-(2,2,2-trifluoroethyl)thieno[3,2-b]pyridine as yellow oil. LCMS: m/z=252 [M+1]⁺

### Step 4. N-(1-methylpiperidin-4-yl)-3-(2,2,2-trifluoroethyl)thieno[3,2-b]pyridin-7-amine

Into a 40-mL reaction vial purged and maintained with an inert atmosphere of nitrogen, was placed 7-chloro-3-(2,2,2-trifluoroethyl)thieno[3,2-b]pyridine (0.70 g, 2.78 mmol), 1-methylpiperidin-4-amine (1.42 g, 12.45 mmol), Chloro[(4,5-bis(diphenylphosphino)-9,9-dimethylxanthene)-2-(2-amino-1,1-biphenyl)]palladium(ii), (0.13 g, 0.14 mmol), Dimethylbisdiphenylphosphinoxanthene (0.14 g, 0.25 mmol), Cs₂CO₃ (2.24 g, 6.89 mmol), 1,4-Dioxane (10 mL). The reaction mixture was stirred at 100° C. overnight. The resulting solution was coo led to room temperature and quenched with water (50 mL) and extracted with DCM (2×50 mL). The organic layers combined and washed with brine (30 mL), dried over anhydrous Na₂SO₄, the residue was concentrated under vacuum. The residue was applied onto a silica gel column eluted with DCM:MeOH (v:v=5:1). This resulted in 819.00 mg (89.26% yield) of N-(1-methylpiperidin-4-yl)-3-(2,2,2-trifluoroethyl)thieno[3,2-b]pyridin-7-amine as brown oil. LCMS: m/z=330 [M+1]⁺

### Step 5. 2-iodo-N-(1-methylpiperidin-4-yl)-3-(2,2,2-trifluoroethyl)thieno[3,2-b]pyridin-7-amine

Into a 40-mL reaction vial purged and maintained with an inert atmosphere of nitrogen, was placed N-(1-methylpiperidin-4-yl)-3-(2,2,2-trifluoroethyl)thieno[3,2-b]pyridin-7-amine (0.61 g, 1.84 mmol), NIS (0.64 g, 2.87 mmol), Trifluoromethanesulfonic acid (5 mL), Acetic acid (5 mL) and stirred at the same temperature for 2 h. The resulting solution was quenched with water (50 mL) and adjust PH=8 with aq·Na₂CO₃, extracted with EA (2×100 mL). The organic layers combined and washed with brine (50 mL), dried over an hydrous Na₂SO₄, the residue was concentrated under vacuum. The residue was applied onto a silica gel column eluted with EA:Hexane (v:v=1:3). This resulted in 802.00 mg (95.27% yield) of 2-iodo-N-(1-methyl piperidin-4-yl)-3-(2,2,2-trifluoroethyl)thieno[3,2-b]pyridin-7-amine as brown oil. LCMS: m/z=456 [M+1]⁺

### Step 6. 2-(3-((2-methoxy-4-(methylsulfonyl)phenyl)amino)prop-1-yn-1-yl)-N-(1-methylpiperidin-4-yl)-3-(2,2,2-trifluoroethyl)thieno[3,2-b]pyridin-7-amine (53)

Into a 40-mL reaction vial purged and maintained with an inert atmosphere of nitrogen, was placed 2-iodo-N-(1-methylpiperidin-4-yl)-3-(2,2,2-trifluoroethyl)thieno[3,2-b]pyridin-7-amine (0.33 g, 0.73 mmol), 2-methoxy-4-(methylsulfonyl)-N-(prop-2-yn-1-yl)aniline (0.32 g, 1.34 mmol), Pd(PPh₃)₂Cl₂ (0.68 g, 0.096 mmol), CuI (90.00 mg, 0.47 mmol), DIEA (0.49 g, 3.85 mmol), DMSO (5 mL). The reaction mixture was stirred at room temperature for 3 h. The resulting solution was quenched with H₂O (50 mL) and extracted with DCM (2×50 mL). The residue was applied onto a C₁₈ column eluted with ACN:H₂O (0.01% TFA) (v:v=1:1), Flow rate: 80 mL/min. This resulted in 102.00 mg (24.46% yield) of 2-(3-((2-methoxy-4-(methylsulfonyl)phenyl)amino)prop-1-yn-1-yl)-N-(1-methylpiperidin-4-yl)-3-(2,2,2-trifluoroethyl)thieno[3,2-b]pyridin-7-amine (53) as yellow solid. LCMS: m/z=567 [M+1]⁺

¹H NMR (400 MHz, DMSO-d₆) δ 8.30 (s, 1H), 8.06 (s, 1H), 7.42 (d, J=7.0 Hz, 1H), 7.25 (t, J=8.0 Hz, 1H), 6.95 (d, J=8.4 Hz, 1H), 6.55 (t, J=6.0 Hz, 1H), 5.74 (d, J=8.9 Hz, 1H), 4.38 (d, J=6.0 Hz, 2H), 4.04 (s, 1H), 3.93-3.73 (m, 5H), 3.09 (d, J=20.3 Hz, 3H), 2.96-2.69 (m, 2H), 2.54-2.49 (m, 3H), 2.31 (s, 2H), 1.84 (s, 2H), 1.55 (s, 2H).

### Example 54

### (3-methoxy-4-((3-(7-(((tetrahydro-1H-pyrrolizin-7a(5H)-yl)methyl)amino)-3-(2,2,2-trifluoroethyl)benzo[b]thiophen-2-yl)prop-2-yn-1-yl)amino)phenyl)dimethylphosphine oxide (54)

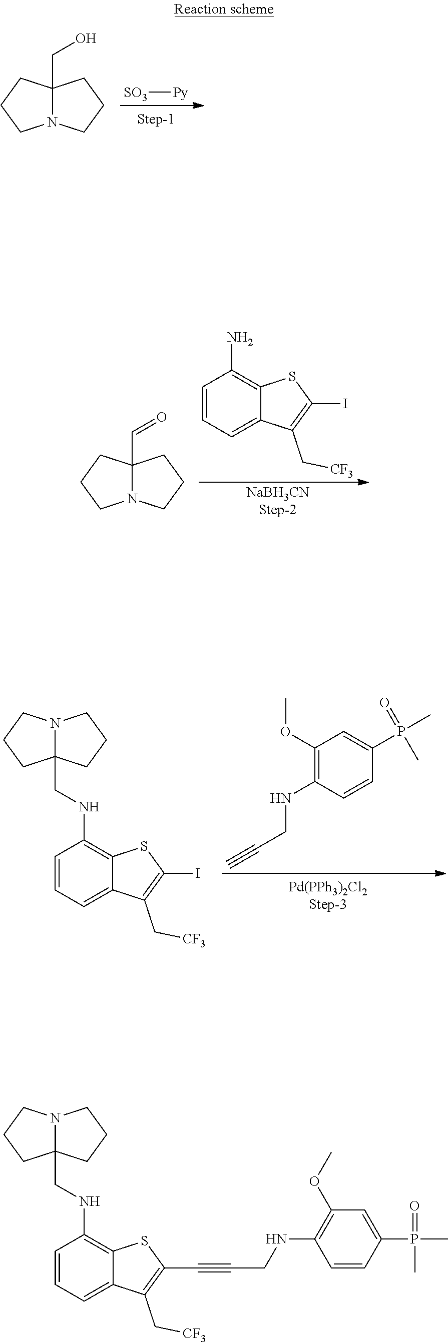

### Experiment Details

### Step 1. tetrahydro-1H-pyrrolizine-7a(5H)-carbaldehyde

Into a 4 mL vail was placed (tetrahydro-1H-pyrrolizin-7a(5H)-yl)methanol (0.504 g, 3.57 mmol), TEA (3.692 g, 36.49 mmol), DMSO (20 mL), DCM (10 mL). The reaction was cooled to 0, and then Pyridine sulfur trioxide (2.295 g, 14.42 mmol) was added. The reaction was stirred overnight at room temperature. LCMS showed the reaction was complete. The reaction was quenched with water (50 mL), extracted with EA (20 mL×2). The aqueous layer was concentrated with vacuum to afford crude tetrahydro-1H-pyrrolizine-7 a(5H)-carbaldehyde (0.192 g, 665.88 μmol, 139.43% yield) as white solid. LCMS: m/z=140[M+1]⁺.

### Step 2. 2-iodo-N-((tetrahydro-1H-pyrrolizin-7a(5H)-yl)methyl)-3-(2,2,2-trifluoroethyl)benzo[b]thiophen-7-amine

Into a 4 mL flask was placed tetrahydro-1H-pyrrolizine-7a(5H)-carbaldehyde (0.099 g, 711.24 μmol), 2-iodo-3-(2,2,2-trifluoroethyl)benzo[b]thiophen-7-amine (0.101 g, 282.81 thyl)benzo[b]action was heated to 150° C. and stirred for 20 min then cooled to room temperature naturally. Sodium cyanoborohydride (0.101 g, 2.36 mmol), acetic acid (0.01 μmol), Ethanol (0.5 mL) was added and stirred for 1.5 h at room temperature. LCMS monitored the reaction was complete. the reaction was purified with C₁₈ column (ACN/water v/v=3/2) to afford 2-iodo-N-((tetrahydro-1H-pyrrolizin-7a(5H)-yl)methyl)-3-(2,2,2-trifluoroethyl)benzo[b]thiophen-7-amine (0.036 g, 74.95 μmol, 26.50% yield) as off-white solid. LCMS: m/z=481[M+1]⁺.

### Step 3. (3-methoxy-4-((3-(7-(((tetrahydro-1H-pyrrolizin-7a(5H)-yl)methyl)amino)-3-(2,2,2-trifluoroethyl)benzo[b]thiophen-2-yl)prop-2-yn-1-yl)amino)phenyl)dimethylphosphine oxide (54)

Into a 4 mL vial purged and maintained with nitrogen atmosphere was placed 3-fluoro-(3-methoxy-4-(prop-2-yn-1-ylamino)phenyl)dimethylphosphine oxide (0.021 g, 88.52 μmol), N-(3-fluoro-1-methylpiperidin-4-yl)-3-methoxy-4-(prop-2-yn-1-ylamino)benzamide (0.028 g, 58.29 μmol), Pd(PPh₃)₂Cl₂ (0.005 g, 7.08 μmol), CuI (0.001 g, 5.25 μmol), TEA (0.018 g, 177.88 μmol), DMF (1 mL), and stirred for 1.5 h at room temperature. LCMS monitored that the reaction was complete. The reaction was quenched with water (2 mL), extracted with EA (2 mL×2). The combined organic layers were washed with water (2 mL) and brine (2 mL) successively, separated, then concentrated with vacuum. The residue was purified with prep-HPLC (Mobile Phase A: water (formic acid), Mobile Phase B: ACN; Flow rate: 70 mL/min; Gradient: 25-45-45% B (2-30-min); 262 nm; RT: 27.501-29.585 min) to afford (3-methoxy-4-((3-(7-(((tetrahydro-1H-pyrrolizin-7a(5H)-yl)methyl)amino)-3-(2,2,2-trifluoroethyl)benzo[b]thiophen-2-yl)prop-2-yn-1-yl)amino)phenyl)dimethylphosphine oxide (54) (2 mg, 3.39 μmol, 5.82% yield) as a white solid. LCMS: m/z=590 [M+1]⁺.

### Example 55

### N-(2-(3-((2-methoxy-4-(methylsulfonyl)phenyl)amino)prop-1-yn-1-yl)-3-(2,2,2-trifluoroethyl)benzo[b]thiophen-7-yl)-1-methylazepan-4-amine (55)

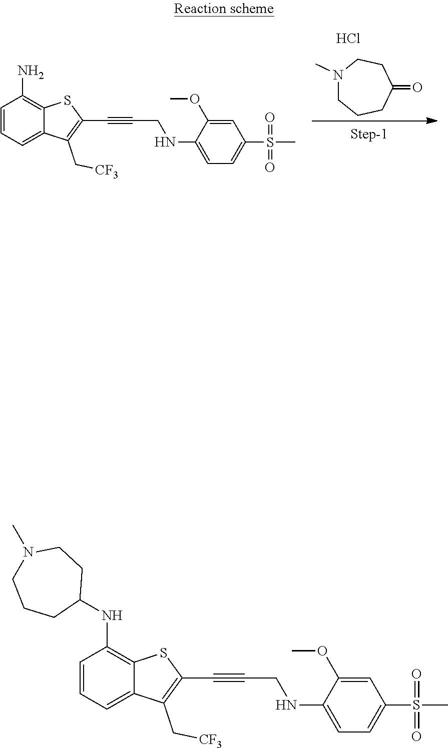

### Experiment Details

### Step 1. N-(2-(3-((2-methoxy-4-(methylsulfonyl)phenyl)amino)prop-1-yn-1-yl)-3-(2,2,2-trifluoroethyl)benzo[b]thiophen-7-yl)-1-methylazepan-4-amine (55)

Into a 4 mL vail was placed 2-(3-((2-methoxy-4-(methylsulfonyl)phenyl)amino)prop-1-yn-1-yl)-3-(2,2,2-trifluoroethyl)benzo[b]thiophen-7-amine (0.092 g, 196.37 μmol), 1-methylazepan-4-one hydrochloride (0.096 g, 586.64 μmol), PhSiH₃ (0.094 g, 868.66 μmol), Dibutyltin dichloride (0.035 g, 115.19 μmol), MeOH (1 mL). The reaction was stirred overnight at room temperature. LCMS showed the reaction was complete. The reaction was quenched with water (2 mL), extracted with EA (2 mL×2). The combined organic layers were washed with water (2 mL) and brine (2 mL) successively, separated, then concentrated with vacuum, then purified with prep-HPLC (Mobile Phase A: water (formic acid), Mobile Phase B: ACN; Flow rate: 70 mL/min; Gradient: 55-85-95% B (2-30-45 min); 269 nm; RT:40.710-43.355 min) to afford N-(2-(3-((2-methoxy-4-(methylsulfonyl)phenyl)amino)prop-1-yn-1-yl)-3-(2,2,2-trifluoroethyl)benzo[b]thiophen-7-yl)-1-methylazepan-4-amine (0.192 g, 665.88 μmol, 139.43% yield) as white solid. LCMS: m/z=580[M+1]⁺.

¹H NMR (400 MHz, DMSO-d₆) δ 7.44-7.36 (m, 1H), 7.31-7.22 (m, 2H), 7.14 (d, J=8.0 Hz, 1H), 6.89 (d, J=8.4 Hz, 1H), 6.57 (d, J=7.8 Hz, 1H), 6.51 (t, J=6.2 Hz, 1H), 5.35 (d, J=8.3 Hz, 1H), 4.38 (d, J=6.3 Hz, 2H), 3.91-3.70 (m, 5H), 3.10 (s, 3H), 2.69-2.56 (m, 1H), 2.49-2.40 (m, 4H), 2.27 (s, 3H), 1.97-1.53 (m, 6H).

### Example 56

### 2-(3-((2-methoxy-4-(methylsulfonyl)phenyl)amino)prop-1-yn-1-yl)-N-((1-methylpiperidin-4-yl)methyl)-3-(2,2,2-trifluoroethyl)benzo[b]thiophen-7-amine (56)

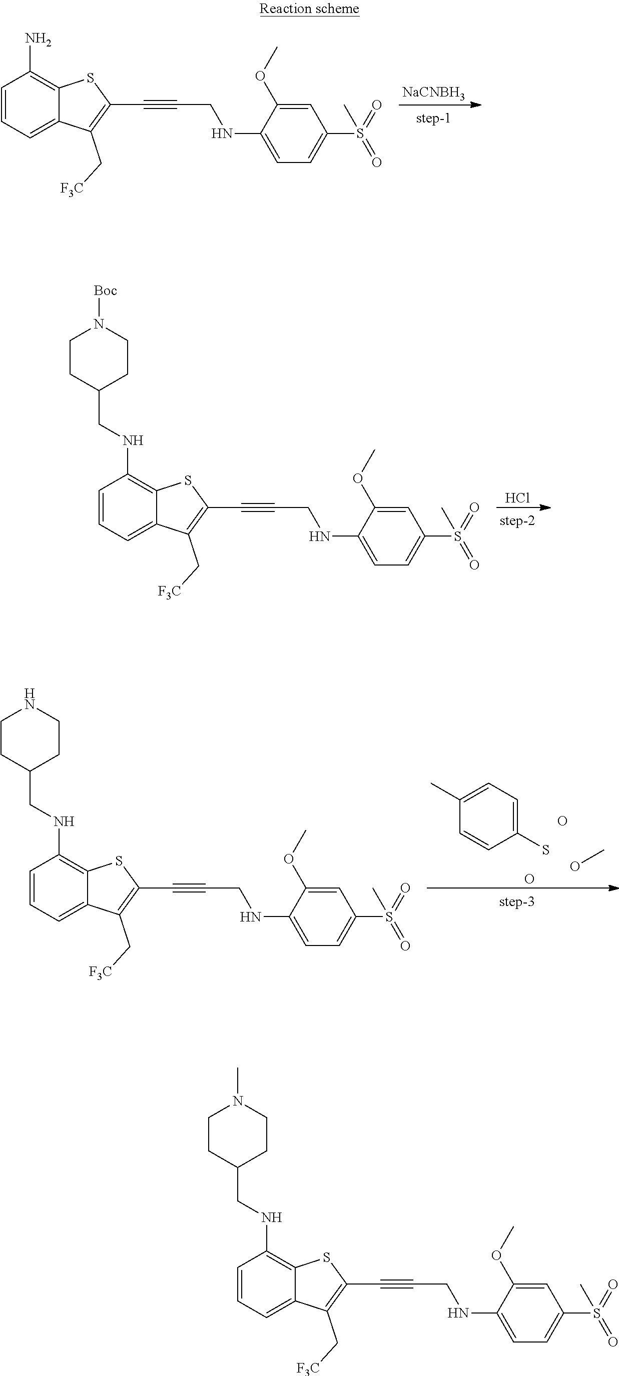

### Experimental Details

### Step 1. tert-butyl 4-(((2-(3-((2-methoxy-4-(methylsulfonyl)phenyl)amino)prop-1-yn-1-yl)-3-(2,2,2-trifluoroethyl)benzo[b]thiophen-7-yl)amino)methyl)piperidine-1-carboxylate

Into a 100-mL three necked bottle purged and maintained with an inert atmosphere of nitrogen, was placed 2-(3-((2-methoxy-4-(methylsulfonyl)phenyl)amino)prop-1-yn-1-yl)-3-(2,2,2-trifluoroethyl)benzo[b]thiophen-7-amine (0.223 g, 0.48 mmol), tert-butyl 4-formylpiperidine-1-carboxylate (0.506 g, 2.37 mmol), NaCNBH₃ (0.199 g, 4.64 mmol), and MeOH (10 mL). The reaction mixture was stirred at 60° C. for 16 h. The resulting solution was added to water (30 mL). The resulting solution was extracted with EA (2×15 mL). The organic layers combined and washed with brine (20 mL), dried over anhydrous Na₂SO₄, the residue was concentrated under vacuum. The crude product purified by washing with HEX/EA (v/v=4/6). This resulted in 0.651 g (205.43% yield) of tert-butyl 4-(((2-(3-((2-methoxy-4-(methylsulfonyl)phenyl)amino)prop-1-yn-1-yl)-3-(2,2,2-trifluoroethyl)benzo[b]thiophen-7-yl)amino)methyl)piperidine-1-carboxylate as red oil. LCMS: m/z=666 [M+1]⁺

### Step 2. 2-(3-((2-methoxy-4-(methylsulfonyl)phenyl)amino)prop-1-yn-1-yl)-N-(piperidin-4-ylmethyl)-3-(2,2,2-trifluoroethyl)benzo[b]thiophen-7-amine

Into a 50-mL flask was placed tert-butyl 4-(((2-(3-((2-methoxy-4-(methylsulfonyl)phenyl)amino)prop-1-yn-1-yl)-3-(2,2,2-trifluoroethyl)benzo[b]thiophen-7-yl)amino)methyl)piperidine-1-carboxylate (0.440 g, 0.66 mmol), HCl (5 mL, 4 M in EA). The mixture was stirred at 20° C. for 1 h. The reaction mixture was purified by C18 column eluted with ACN/H₂O (v/v=1/1). This resulted in 0.249 g (66.61% yield) of 2-(3-((2-methoxy-4-(methylsulfonyl)phenyl)amino)prop-1-yn-1-yl)-N-(piperidin-4-ylmethyl)-3-(2,2,2-trifluoroethyl)benzo[b]thiophen-7-amine as light yellow solid. LCMS: m/z=566 [M+1]⁺.

### Step 3. 2-(3-((2-methoxy-4-(methylsulfonyl)phenyl)amino)prop-1-yn-1-yl)-N-((1-methylpiperidin-4-yl)methyl)-3-(2,2,2-trifluoroethyl)benzo[b]thiophen-7-amine (56)

Into a 50-mL flask was placed 2-(3-((2-methoxy-4-(methylsulfonyl)phenyl)amino)prop-1-yn-1-yl)-N-(piperidin-4-ylmethyl)-3-(2,2,2-trifluoroethyl)benzo[b]thiophen-7-amine (0.082 g, 0.14 mmol), Methyl p-toluenesulfonate (0.313 g, 1.68 mmol), K₂CO₃ (0.035 g, 0.25 mmol), THF (2 mL). The mixture was stirred at 20° C. for 1 h. LCMS showed that the reaction was completed. The resulting solution was added to sodium carbonate aqueous solution (50 mL) and extracted with EA (3×50 mL). The organic layers combined and washed with brine (50 mL), dried over anhydrous Na₂SO₄, the residue was concentrated under vacuum. The mixture was purified by preparative HPLC (Mobile Phase A: water (10 mmoL/L ammonium hydroxide), Mobile Phase B: ACN; Flow rate: 40 mL/min; Gradient: 30-50-50% B (2-30-60 min); 247 nm; RT: 26.910-28.635 min). This resulted in 0.003 g (3.57% yield) of 2-(3-((2-methoxy-4-(methylsulfonyl)phenyl)amino)prop-1-yn-1-yl)-N-((1-methylpiperidin-4-yl)methyl)-3-(2,2,2-trifluoroethyl)benzo[b]thiophen-7-amine (56) as off white solid. LCMS: m/z=580 [M+1]⁺.

### Example 57

### 4-((3-(4-(((Z)-3-fluoro-1-methylpiperidin-4-yl)amino)-1-(2,2,2-trifluoroethyl)-1H-indol-2-yl)prop-2-yn-1-yl)amino)-3-methoxy-N-methylbenzamide (57)

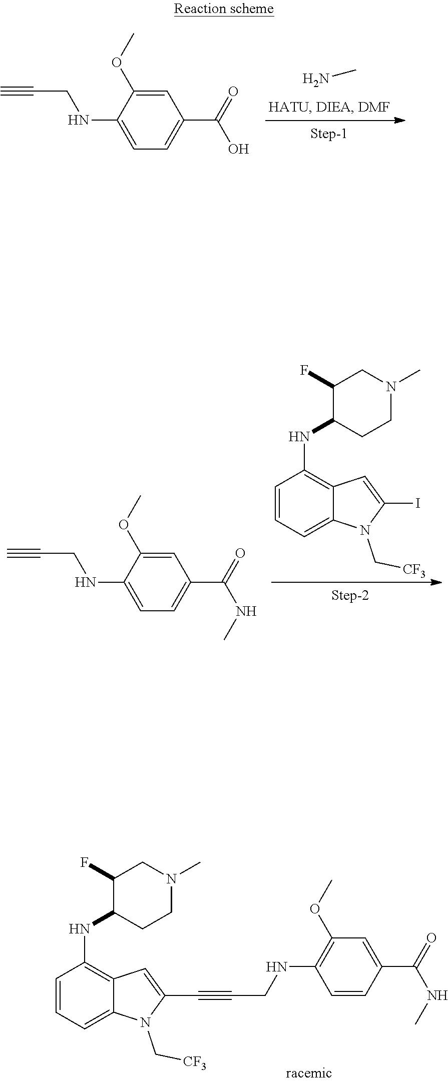

### Experimental Details

### Step 1. 3-methoxy-N-methyl-4-(prop-2-yn-1-ylamino)benzamid

Into a 100-mL three necked bottle purged and maintained with an inert atmosphere of nitrogen, was placed 3-methoxy-4-(prop-2-yn-1-ylamino)benzoic acid (1.50 g, 7.31 mmol), methanamine (1.59 g, 23.57 mmol), HATU (4.51 g, 11.87 mmol), DIEA (4.24 g, 32.86 mmol), DMF (30 mL). The reaction mixture was stirred at room temperature overnight. The resulting solution was added to water (100 mL). The resulting solution was extracted with EA (2×150 mL). The organic layers combined and washed with brine (20 mL), dried over anhydrous Na₂SO₄, the residue was concentrated under vacuum. The residue was applied onto a silica gel column eluted with EA:hexane (v:v=1:1). This resulted in 1.50 g (94.02% yield) of 3-methoxy-N-methyl-4-(prop-2-yn-1-ylamino)benzamide as yellow oil. LCMS: m/z=219 [M+1]⁺

### Step 2. 4-((3-(4-(((Z)-3-fluoro-1-methylpiperidin-4-yl)amino)-1-(2,2,2-trifluoroethyl)-1H-indol-2-yl)prop-2-yn-1-yl)amino)-3-methoxy-N-methylbenzamide (57)

Into a 100-mL three necked bottle purged and maintained with an inert atmosphere of nitrogen, was placed N—((Z)-3-fluoro-1-methylpiperidin-4-yl)-2-iodo-1-(2,2,2-trifluoroethyl)-1H-indol-4-amine (1.41 g, 3.11 mmol), 3-methoxy-N-methyl-4-(prop-2-yn-1-ylamino)benzamide (1.61 g, 7.40 mmol), CuI (0.16 g, 0.88 mmol), Pd(PPh₃)₂Cl₂ (0.25 g, 0.36 mmol), DIEA (1.40 g, 10.83 mmol) and DMSO (20 mL). The reaction mixture was stirred at room temperature for 2 h. LCMS showed that the reaction was completed. The resulting solution was added to water (100 mL) and extracted with EA (3×100 mL). The organic layers combined and washed with brine (50 mL), dried over anhydrous Na₂SO₄, the residue was concentrated under vacuum. The residue was applied onto a silica gel column eluted with ACN:H₂O (v:v=1:1), Flow rate: 100 mL/min. This resulted in 0.55 g (32.76% yield) of 4-((3-(4-(((Z)-3-fluoro-1-methylpiperidin-4-yl)amino)-1-(2,2,2-trifluoroethyl)-1H-indol-2-yl)prop-2-yn-1-yl)amino)-3-methoxy-N-methylbenzamide (57) as yellow solid. LCMS: m/z=546 [M+1]⁺

¹H NMR (400 MHz, DMSO-d₆) δ 8.10 (d, J=4.5 Hz, 1H), 7.50-7.29 (m, 2H), 7.17 (s, 1H), 7.00 (t, J=8.0 Hz, 1H), 6.75 (t, J=8.7 Hz, 2H), 6.27-6.23 (m, 1H), 5.99-5.94 (m, 1H), 5.49 (d, J=8.6 Hz, 1H), 4.89-4.85 (m, 3H), 4.31 (d, J=6.2 Hz, 2H), 3.84 (s, 3H), 3.59-3.55 (m, 1H), 3.06-3.02 (m, 1H), 2.79-2.70 (m, 4H), 2.31-2.02 (m, 5H), 1.95-1.90 (m, 1H), 1.69-1.65 (m, 1H).

### Example 58

### 4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-N-hydroxy-3-methoxybenzamide(racemic)(58)

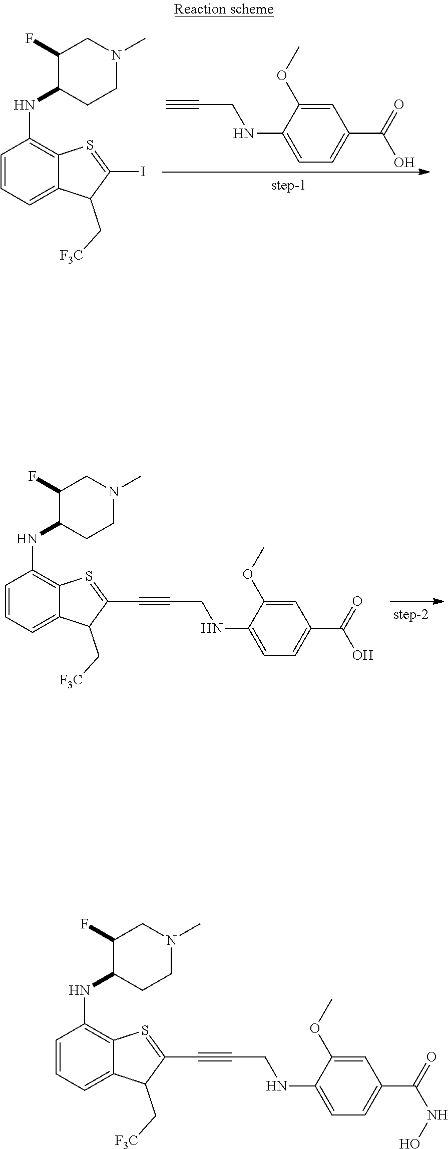

### Experiment Details

### Step 1. 4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-methoxybenzoic acid(racemic

Into a 8 mL vial was placed 3-methoxy-4-(prop-2-yn-1-ylamino)benzoic acid (0.06 g, 0.31 mmol), (Z)-3-fluoro-N-(2-iodo-3-(2,2,2-trifluoroethyl)benzo[b]thiophen-7-yl)-1-methylpiperidin-4-amine(racemic) (0.05 g, 0.22 mmol), bis(triphenylphosphine)palladium(II) chloride (0.03 g, 46.75 μmol), CuI (0.02 g, 78.76 μmol), DIEA (0.09 g, 0.71 mmol), Methyl sulfoxide (2 mL). The reaction was stirred at R.T for 1 h under nitrogen. The reaction was quenched with water (20 mL), extracted with EA (3×30 mL). The organic layers were combined, washed with brine (50 mL), dried over anhydrous sodium sulfate, filtered and concentrated under vacuum. The residue purified by flash-C18 eluted with ACN/water (v/v=1/1) to provide the desired product. This resulted in 0.274 g (crude) of 4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-methoxybenzoic acid as brown oil. LCMS: m/z=550 [M+1]⁺.

### Step 2. 4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-N-hydroxy-3-methoxybenzamide(racemic)(58)

Into a 40 mL vial placed 4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-methoxybenzoic(racemic) (0.26 g, 0.48 mmol), Hydroxylammoniumchloride (0.25 g, 0.35 mmol), n-Propylphosphonic cyclic anhydride (0.66 g, 0.10 mmol), DIEA (0.83 g, 0.64 m mol), DMF (10 mL). The reaction was stirred at R.T overnight. The reaction was quenched with water (20 mL), extracted with EA (3×30 mL). The organic layers were combined, washed with brine (50 mL), dried over anhydrous sodium sulfate, filtered and concentrated under vacuum. The mixture was purified by preparative HPLC (Mobile Phase A: water (10 mmol/L ammonium hydroxide), Mobile Phase B:MeOH; Flow rate: 70 mL/min; Gradient: 50-80-100% B (2-30-60 min); 269 nm; RT: 33.84-34.62) to provide the desired product. This resulted in 0.015 g (5.55% yield) of 4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-N-hydroxy-3-methoxybenzamide(racemic) (58) as brown solid. LCMS: m/z=565 [M+1]⁺.

### Example 59

### 4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-N-(2-hydroxy-3-methoxypropyl)-3-methoxybenzamide (59)

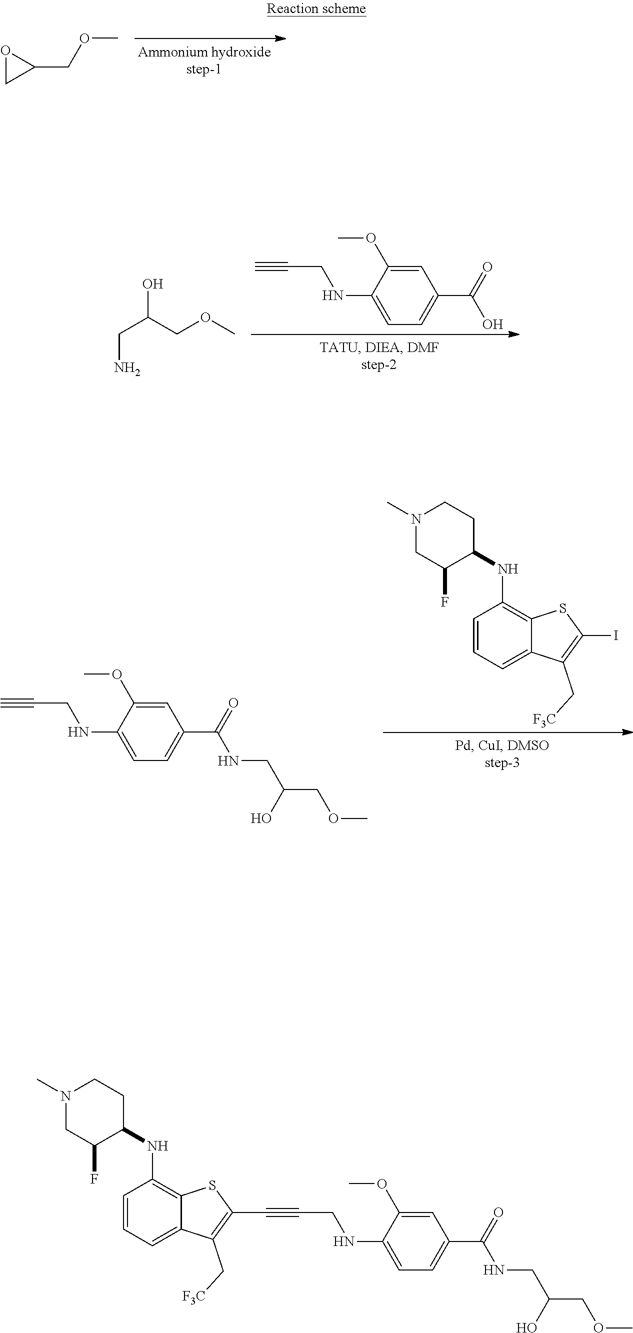

### Experimental Details

### Step 1. 1-amino-3-methoxypropan-2-ol

Into a 25-mL round-bottom flask purged and maintained with an inert atmosphere of nitrogen, was placed 2-(methoxymethyl)oxirane (0.953 g, 10.81 mmol), Ammonium hydroxide (5 mL). The reaction mixture was stirred at 90° C. for 16 h The mixture was concentrated to afford the crude product. This resulted in 1.579 g (138.84% yield) of 1-amino-3-methoxypropan-2-ol as colourless oil. LCMS: m/z=106 [M+1]⁺.

### Step 2. N-(2-hydroxy-3-methoxypropyl)-3-methoxy-4-(prop-2-yn-1-ylamino)benzamide

Into a 25-mL round-bottom flask purged and maintained with an inert atmosphere of nitrogen, was placed 3-methoxy-4-(prop-2-yn-1-ylamino)benzoic acid (0.302 g, 1.47 mmol), 1-amino-3-methoxypropan-2-ol (1.521 g, 14.46 mmol), DIEA (0.404 g, 3.12 mmol), TATU (0.469 g, 1.23 mmol), DMF (10 mL). The reaction mixture was stirred at room temperature for 1 h. The reaction mixture was filtered through celite pad and celite pa d was washed with ACN (3×20 mL). The filterate was then concentrated under reduced pressure to afford the crude product. The crude was purified by C18 column eluted with ACN/H₂O (v/v=1/3), This resulted in 0.298 g, (69.26% yield) of N-(2-hydroxy-3-methoxypropyl)-3-methoxy-4-(prop-2-yn-1-ylamino)benzamide as brown oil. LCMS: m/z=293 [M+1]⁺.

### Step 3. 4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-N-(2-hydroxy-3-methoxypropyl)-3-methoxybenzamide (59)

Into a 10-mL round-bottom flask was placed N-(2-hydroxy-3-methoxypropyl)-3-methoxy-4-(prop-2-yn-1-ylamino)benzamide (0.285 g, 974.92 mmol), (Z)-3-fluoro-N-(2-iodo-3-(2,2,2-trifluoroethyl)benzo[b]thiophen-7-yl)-1-methylpiperidin-4-amine ((0.081 g, 171.50 μmol), Pd(PPh₃)₂Cl₂ (0.037 g, 52.41 μmol), CuI (0.024 g, 126.01 μmol), DIEA (00.075 g, 580.30 μmol), methyl sulfoxide (3 mL). The reaction was stirred under nitrogen atmosphere at RT for 2 h. The reaction mixture was filtered through celite pad and celite pad was washed with ACN (3×20 mL). The filterate was then concentrated under reduced pressure to afford the crude product. The crude product was purified by preparative HPLC (Mobile Phase A: water (0.1% ammonium hydroxide), Mobile Phase B: ACN; Flow rate: 70 mL/min; Gradient: 55-80-100% B (2-30-60 min); 266 nm; RT: 32.375-34.406) to provide the desired product. This resulted in 0.060 g (54.94% yield) of 4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-N-(2-hydroxy-3-methoxypropyl)-3-methoxybenzamide (59) as white solid. LCMS: m/z=637 [M+1]⁺.

¹H NMR (400 MHz, DMSO-d₆) δ 8.04 (t, J=5.7 Hz, 1H), 7.37 (d, J=8.3 Hz, 1H), 7.29 (s, 1H), 7.22-7.09 (m, 2H), 6.69-6.66 (m, 2H), 5.95 (t, J=6.4 Hz, 1H), 5.06 (d, J=8.5 Hz, 1H), 4.88 (d, J=4.8 Hz, 1H), 4.70 (d, J=49.5 Hz, 1H), 4.26 (d, J=6.1 Hz, 2H), 3.79-3.50 (m, 8H), 3.21 (s, 2H), 3.17 (s, 3H), 3.10-3.04 (m, 1H), 2.94 (t, J=11.2 Hz, 1H), 2.70 (d, J=10.8 Hz, 1H), 2.18 (d, J=13.1 Hz, 1H), 2.09 (s, 3H), 1.99 (t, J=11.5 Hz, 1H), 1.92-1.79 (m, 1H), 1.62 (d, J=12.6 Hz, 1H).

### Example 60

### 5-((3-(2,2,2-trifluoroethyl)-7-(((Z)-3-fluoro-1-methylpiperidin-4-yl)amino)benzo[b]thiophen-2-yl)prop-2-yn-1-yl)amino)-4-methoxy-N-methylpicolinamide (racemic) (60)

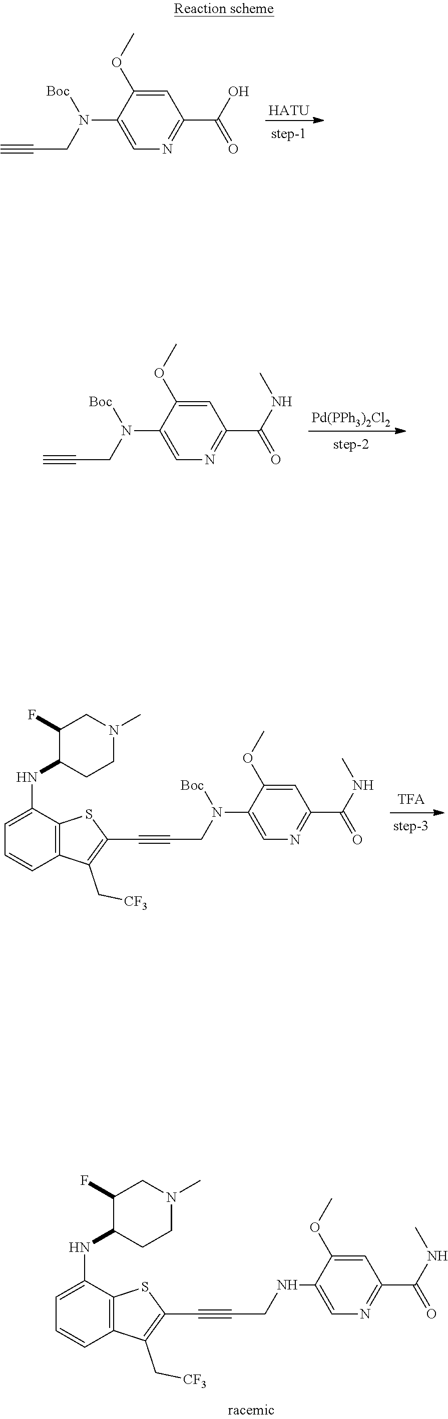

### Experimental Details

### Step 1. tert-butyl (4-methoxy-6-(methylcarbamoyl)pyridin-3-yl)(prop-2-yn-1-yl)carbamate

Into a 100-mL three necked bottle purged and maintained with an inert atmosphere of nitrogen, was placed 5-((tert-butoxycarbonyl)(prop-2-yn-1-yl)amino)-4-methoxypicolinic acid (0.910 g, 2.97 mmol), Methylamine hydrochloride (1.844 g, 27.31 mmol), HATU (1.890 g, 4.97 mmol), DIEA (1.808 g, 13.99 mmol), DMF (10 mL). The reaction mixture was stirred at 25° C. for 1 h. The reaction mixture was purified by C18 column eluted with ACN/H₂O (v/v=1/1). This resulted in 0.642 g (67.68% yield) of tert-butyl (4-methoxy-6-(methylcarbamoyl)pyridin-3-yl)(prop-2-yn-1-yl)carbamate as red solid. LCMS: m/z=320 [M+1]⁺.

### Step 2. tert-butyl (3-(3-(2,2,2-trifluoroethyl)-7-(((Z)-3-fluoro-1-methylpiperidin-4-yl)amino)benzo[b]thiophen-2-yl)prop-2-yn-1-yl)(4-methoxy-6-(methylcarbamoyl)pyridin-3-yl)carbamate (racemic)

Into a 50-mL three necked bottle and maintained with an inert atmosphere of nitrogen, was placed (Z)—N-(3-(2,2,2-trifluoroethyl)-2-iodobenzo[b]thiophen-7-yl)-3-fluoro-1-methylpiperidin-4-amine (racemic) (0.090 g, 0.19 mmol), tert-butyl (4-methoxy-6-(methylcarbamoyl)pyridin-3-yl)(prop-2-yn-1-yl)carbamate (0.107 g, 0.34 m mol), CuI (0.022 g, 0.12 mmol), Pd(PPh₃)₂Cl₂ (0.050 g, 0.71 mmol), DIEA (0.078 g, 0.61 mmol) and DMSO (5 mL). The reaction mixture was stirred at room temperature for 1 h. LCMS showed that the reaction was completed. The reaction mixture was purified by C18 column eluted with ACN/H₂O (v/v=6/4). This resulted in 0.073 g (57.72% yield) of tert-butyl (3-(3-(2,2,2-trifluoroethyl)-7-(((Z)-3-fluoro-1-methylpiperidin-4-yl)amino)benzo[b]thiophen-2-yl)prop-2-yn-1-yl)(4-methoxy-6-(methylcarbamoyl)pyridin-3-yl)carbamate (race mic) as red solid. LCMS: m/z=664 [M+1]⁺.

### Step 3. 5-((3-(2,2,2-trifluoroethyl)-7-(((Z)-3-fluoro-1-methylpiperidin-4-yl)amino)benzo[b]thiophen-2-yl)prop-2-yn-1-yl)amino)-4-methoxy-N-methylpicolinamide (racemic) (60)

Into a 50-mL flask was placed tert-butyl (3-(3-(2,2,2-trifluoroethyl)-7-(((Z)-3-fluoro-1-methylpiperidin-4-yl)amino)benzo[b]thiophen-2-yl)prop-2-yn-1-yl)(4-methoxy-6-(methylcarbamoyl)pyridin-3-yl)carbamate (racemic) (0.075 g, 0.12 mmol), DCM (2 mL), TFA (1 mL). The mixture was stirred at 20° C. for 1 h. LCMS showed that the reaction was completed. The resulting solution was added to sodium carbonate aqueous solution (50 mL) and extracted with EA (3×50 mL). The organic layers combined and washed with brine (50 mL), dried over anhydrous Na₂SO₄, the residue was concentrated under vacuum. The mixture was purified by preparative HPLC (Mobile Phase A: water (10 mmoL/L ammonium hydroxide), Mobile Phase B: ACN; Flow rate: 40 mL/min; Gradient: 30-50-50% B (2-30-60 min); 247 nm; RT: 26.910-28.635 min). This resulted in 0.010 g (15.71% yield) of 5-((3-(3-(2,2,2-trifluoroethyl)-7-(((Z)-3-fluoro-1-methylpiperidin-4-yl)amino)benzo[b]thiophen-2-yl)prop-2-yn-1-yl)amino)-4-methoxy-N-methylpicolinamide (racemic) (60) as off white solid. LCMS: m/z=564 [M+1]⁺.

¹H NMR (400 MHz, DMSO) δ 8.40 (d, J=4.4 Hz, 1H), 7.99 (s, 1H), 7.51 (s, 1H), 7.36-7.12 (m, 2H), 6.78 (d, J=7.5 Hz, 1H), 6.20 (t, J=6.1 Hz, 1H), 5.16 (d, J=8.2 Hz, 1H), 4.80 (d, J=49.7 Hz, 1H), 4.42 (d, J=6.2 Hz, 2H), 3.94 (s, 3H), 3.85-3.73 (m, 2H), 3.65 (d, J=28.9 Hz, 1H), 3.03 (t, J=11.1 Hz, 1H), 2.79 (d, J=4.4 Hz, 3H), 2.71-2.55 (m, 1H), 2.34-2.22 (m, 1H), 2.16 (d, J=24.7 Hz, 3H), 2.09 (t, J=11.7 Hz, 1H), 2.01-1.87 (m, 1H), 1.72 (d, J=10.8 Hz, 1H).

### Example 61

### 5-((3-(3-(2,2,2-trifluoroethyl)-7-(((Z)-3-fluoro-1-methylpiperidin-4-yl)amino)benzo[b]thiophen-2-yl)prop-2-yn-1-yl)amino)-4-methoxypicolinamide (racemic) (61)

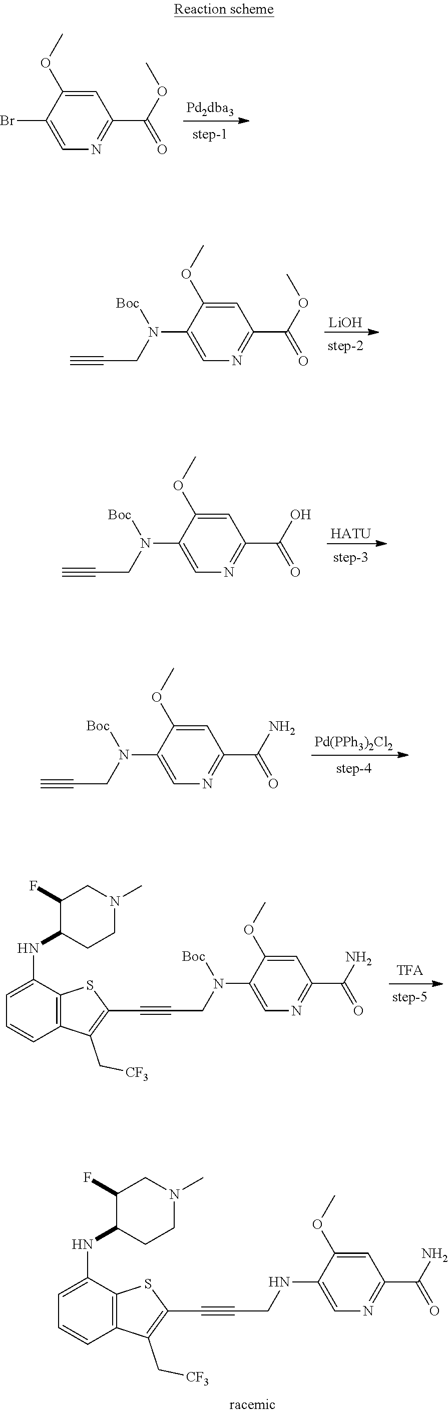

### Experimental Details

### Step 1. methyl 5-((tert-butoxycarbonyl)(prop-2-yn-1-yl)amino)-4-methoxypicolinate

Into a 100-mL three necked bottle purged and maintained with an inert atmosphere of nitrogen, was placed methyl 5-bromo-4-Methoxypyridine-2-carboxylate (5.044 g, 20.50 mmol), N-Boc-propargylamine (4.008 g, 25.83 mmol), Pd₂dba₃ (2.797 g, 3.05 mmol), Cs₂CO₃ (13.140 g, 40.33 mmol), DIOX (50 mL). The reaction mixture was stirred at 100° C. for 6 h. The resulting solution was added to water (300 mL). The resulting solution was extracted with EA (2×150 mL). The organic layers combined and washed with brine (200 mL), dried over anhydrous Na₂SO₄, the residue was concentrated under vacuum. The crude product was purified by washing with HEX/EA (v/v=3/7). This resulted in 1.848 g (28.14% yield) of methyl 5-((tert-butoxycarbonyl)(prop-2-yn-1-yl)amino)-4-methoxypicolinate as yellow oil. LCMS: m/z=321 [M+1]⁺.

### Step 2. 5-((tert-butoxycarbonyl)(prop-2-yn-1-yl)amino)-4-methoxypicolinic acid

Into a 50-mL flask was placed methyl 5-((tert-butoxycarbonyl)(prop-2-yn-1-yl)amino)-4-methoxypicolinate (0.842 g, 2.63 mmol), LiOH (1.070 g, 44.68 mmol), MeOH (10 mL), H₂O (10 mL), THF (10 mL). The reaction mixture was stirred at 60° C. for 2 h. LCMS showed that the reaction was completed. The reaction mixture was purified by C18 column eluted with ACN/H₂O (v/v=6/4). This resulted in 0.998 g (103.95% yield) of 5-((tert-butoxycarbonyl)(prop-2-yn-1-yl)amino)-4-methoxypicolinic acid as red solid. LCMS: m/z=307 [M+1]⁺.

### Step 3. tert-butyl (6-carbamoyl-4-methoxypyridin-3-yl)(prop-2-yn-1-yl)carbamate

Into a 100-mL three necked bottle purged and maintained with an inert atmosphere of nitrogen, was placed 5-((tert-butoxycarbonyl)(prop-2-yn-1-yl)amino)-4-methoxypicolinic acid (0.532 g, 1.74 mmol), NH₄Cl (0.718 g, 13.42 mmol), HATU (0.823 g, 2.16 mmol), DIEA (0.672 g, 5.20 mmol), DMF (10 mL). The reaction mixture was stirred at 25° C. for 1 h. The reaction mixture was purified by C18 column eluted with ACN/H₂O (v/v=1/1). This resulted in 0.262 g (49.41% yield) of tert-butyl (6-carbamoyl-4-methoxypyridin-3-yl) (prop-2-yn-1-yl)carbamate as red oil. LCMS: m/z=306 [M+1]⁺.

### Step 4. tert-butyl (6-carbamoyl-4-methoxypyridin-3-yl)(3-(3-(2,2,2-trifluoroethyl)-7-(((Z)-3-fluoro-1-methyl piperidin-4-yl)amino)benzo[b]thiophen-2-yl)prop-2-yn-1-yl)carbamate

Into a 50-mL three necked bottle and maintained with an inert atmosphere of nitrogen, was placed (Z)—N-(3-(2,2,2-trifluoroethyl)-2-iodobenzo[b]thiophen-7-yl)-3-fluoro-1-methylpiperidin-4-amine (racemic) (0.093 g, 0.19 mmol), tert-butyl (6-carbamoyl-4-methoxypyridin-3-yl)(prop-2-yn-1-yl)carbamate (0.093 g, 0.31 mmol), CuI (0.034 g, 0.18 mmol), Pd(PPh₃)₂Cl₂ (0.028 g, 0.40 mmol), DIEA (0.033 g, 0.26 mmol) and DMSO (5 mL). The reaction mixture was stirred at room temperature for 1 h. LCMS showed that the reaction was completed. The reaction mixture was purified by C18 column eluted with ACN/H₂O (v/v=6/4). This resulted in 0.096 g (75.04% yield) of tert-butyl (6-carbamoyl-4-methoxypyridin-3-yl)(3-(3-(2,2,2-trifluoroethyl)-7-(((Z)-3-fluoro-1-methylpiperidin-4-yl)amino)benzo[b]thiophen-2-yl)prop-2-yn-1-yl)carbamate (racemic) as red oil. LCMS: m/z=650 [M+1]⁺.

### Step 5. 5-((3-(3-(2,2,2-trifluoroethyl)-7-(((Z)-3-fluoro-1-methylpiperidin-4-yl)amino)benzo[b]thiophen-2-yl)prop-2-yn-1-yl)amino)-4-methoxypicolinamide (racemic) (61)

Into a 50-mL flask was placed tert-butyl (6-carbamoyl-4-methoxypyridin-3-yl)(3-(3-(2,2,2-trifluoroethyl)-7-(((Z)-3-fluoro-1-methylpiperidin-4-yl)amino)benzo[b]thiophen-2-yl)prop-2-yn-1-yl)carbamate (racemic) (0.100 g, 0.15 mmol), DCM (2 mL), TFA (1 mL). The mixture was stirred at 25° C. for 1 h. LCMS showed that the reaction was completed. The resulting solution was added to sodium carbonate aqueous solution (50 mL) and extracted with EA (3×50 mL). The organic layers combined and washed with brine (50 mL), dried over anhydrous Na₂SO₄, the residue was concentrated under vacuum. The mixture was purified by preparative HPLC (Mobile Phase A: water (10 mmoL/L ammonium hydroxide), Mobile Phase B: ACN; Flow rate: 40 mL/min; Gradient: 10-40-40% B (2-32-60 min); 239 nm; RT: 19.473-20.930 min). This resulted in 0.012 g (14.18% yield) of 5-((3-(3-(2,2,2-trifluoroethyl)-7-(((Z)-3-fluoro-1-methylpiperidin-4-yl)amino)benzo[b]thiophen-2-yl)prop-2-yn-1-yl)amino)-4-methoxypicolinamide (racemic) (61) as off white solid. LCMS: m/z=550 [M+1]⁺.

¹H NMR (400 MHz, DMSO) δ 8.00 (s, 1H), 7.77 (s, 1H), 7.52 (s, 1H), 7.31-7.21 (dd, m, 2H), 6.78 (d, J=7.4 Hz, 1H), 6.22 (t, J=6.2 Hz, 1H), 5.16 (d, J=8.3 Hz, 1H), 4.79 (d, J=49.3 Hz, 1H), 4.42 (d, J=6.1 Hz, 2H), 3.93 (s, 3H), 3.86-3.73 (m, 2H), 3.72-3.56 (m, 1H), 3.07-2.97 (m, 1H), 2.79 (d, J=10.4 Hz, 1H), 2.70-2.55 (m, 1H), 2.34-2.23 (m, 1H), 2.18 (s, 3H), 2.06 (d, J=11.6 Hz, 1H), 1.94 (d, J=9.6 Hz, 1H), 1.71 (d, J=10.2 Hz, 1H).

### Example 62

### 5-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-6-methoxy-N-methylpicolinamide (racemic) (62)

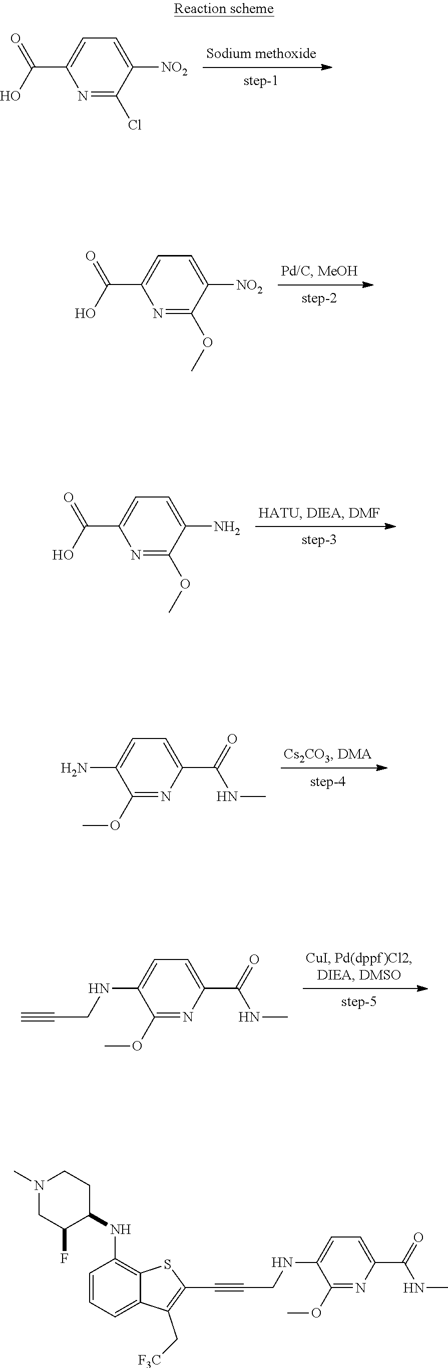

### Experiment Details

### Step 1. 6-methoxy-5-nitropicolinic acid

Into a 20-mL sealed tube was placed 6-chloro-5-nitropicolinic acid (1.022 g, 5.05 mmol), Sodium methoxide (2.786 g, 51.57 mmol), MeOH (10 mL). The reaction mixture was stirred at room temperature for 12 h. The mixture was extracted with EA/H₂O (50 mL×2). The combined organic layers were washed with brine (30 mL), dried over anhydrous Na₂SO₄ and concentrated under vacuum. This resulted in 0.757 g (75.72%) of 6-methoxy-5-nitropicolinic acid as yellow solid. LCMS: m/z=199[M+1]⁺

### Step 2. 5-amino-6-methoxypicolinic acid

To a solution of 6-methoxy-5-nitropicolinic acid (0.725 g, 3.66 mmol) in 10 mL MeOH, 10% Pd/C (0.142 g, 1.33 mmol) was added. The mixture was stirred under hydrogen atmospheric for 12 h and the catalyst was removed by filtration. The filtrate was concentrated under vacuum. This resulted in 0.676 g (crude) of 5-amino-6-methoxypicolinic acid as yellow oil. LCMS: m/z=169 [M+1]⁺

### Step 3. 5-amino-6-methoxy-N-methylpicolinamide

Into a 20-mL sealed tube was placed 5-amino-6-methoxypicolinic acid (0.238 g, 1.42 mmol), Methylamine hydrochloride (0.537 g, 7.95 mmol), HATU (0.859 g, 2.25 mmol), DIEA (1.966 g, 15.21 mmol) and DMF (5 mL). The reaction mixture was stirred at room temperature for 1 h. The mixture was purified with C18 column eluted with ACN/water (v/v=1/5) to afford 0.424 g (crude) of 5-amino-6-methoxy-N-methylpicolinamide as yellow oil. LCMS: m/z=182 [M+1]⁺.

### Step 4. 6-methoxy-N-methyl-5-(prop-2-yn-1-ylamino)picolinamide

Into a 20-mL sealed tube was placed 5-amino-6-methoxy-N-methylpicolinamide (0.336 g, 1.85 mmol), 3-Bromopropyne (0.524 g, 4.40 mmol), Cs₂CO₃ (1.315 g, 4.04 mmol), DMF (10 mL). The reaction mixture was stirred at 100° C. for 12 h. The mixture was purified with C18 column eluted with ACN/water (v/v=1/4) to afford 0.058 g (14.27%) of 6-methoxy-N-methyl-5-(prop-2-yn-1-ylamino)picolinamide as yellow oil. LCMS: m/z=220 [M+1]⁺.

### Step 5. 5-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-6-methoxy-N-methylpicolinamide (racemic) (62)

Into a 8-mL sealed tube purged and maintained with an inert atmosphere of nitrogen, was placed (Z)-3-fluoro-N-(2-iodo-3-(2,2,2-trifluoroethyl)benzo[b]thiophen-7-yl)-1-methylpiperidin-4-amine (racemic) (0.079 g, 0.17 mmol), 6-methoxy-N-methyl-5-(prop-2-yn-1-ylamino)picolinamide (0.055 g, 0.25 mmol), Pd(dppf)Cl₂ (0.019 g, 0.03 mmol), CuI (0.014 g, 0.07 mmol), DIEA (0.031 g, 0.24 mmol), DMSO (1 mL). The reaction mixture was stirred at 50° C. for 1 h. The mixture was purified by preparative HPLC (Mobile Phase A: water (10 mmoL/L ammonium hydroxide), Mobile Phase B: MeCN; Flow rate: 60 mL/min; Gradient: 50-80-95% B (2-30-60 min) 269 nm; RT: 36.143-38.074 min) to afford 5-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-6-methoxy-N-methylpicolinamide (racemic) (62) (0.025 g, 26.52% yield) as white solid. LCMS: m/z=564 [M+1]⁺

¹H NMR (400 MHz, DMSO) δ 8.19 (d, J=4.8 Hz, 1H), 7.54 (d, J=7.6 Hz, 1H), 7.33-7.23 (m, 1H), 7.20 (d, J=7.6 Hz, 1H), 7.06 (d, J=8.0 Hz, 1H), 6.77 (d, J=7.4 Hz, 1H), 6.45-6.36 (m, 1H), 5.18 (d, J=8.6 Hz, 1H), 4.79 (d, J=49.2 Hz, 1H), 4.35 (d, J=6.0 Hz, 2H), 4.02 (s, 3H), 3.91-3.74 (m, 2H), 3.64 (d, J=24.0 Hz, 1H), 3.12-2.95 (m, 1H), 2.80 (d, J=4.8 Hz, 4H), 2.37-2.23 (m, 1H), 2.18 (s, 3H), 2.12-2.03 (m, 1H), 2.03-1.87 (m, 1H), 1.71 (d, J=9.6 Hz, 1H).

### Example 63

### 5-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-6-methoxypicolinamide(racemic) (63)

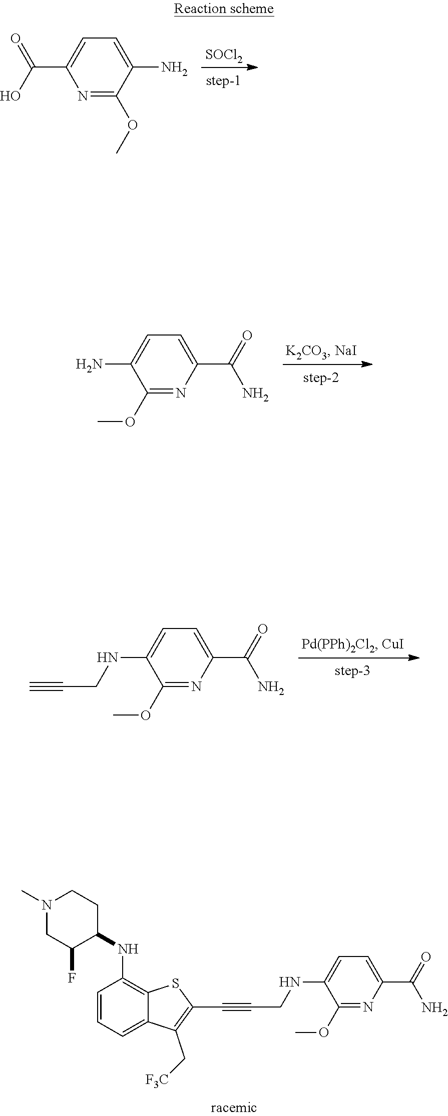

### Experimental Details

### Step 1. 5-amino-6-methoxypicolinamide

Into a 8-mL three necked bottle purged and maintained with an inert atmosphere of nitrogen, was placed 5-amino-6-methoxypicolinic acid (0.200 g, 1.19 mmol), thionyl chloride (2 mL). The reaction mixture was stirred at 80° C. for 3 h. the mixture was concentrated under vacuum and dissolved with THF (20 mL). Then ammonia (7 M in MeOH, 20 mL) was added at 0° C. The mixture was stirred at 20° C. for 0.5 h. The resulting solution was added to water (10 mL) and extracted with EA (2×10 mL). The organic layer was combined and washed with brine (10 mL), dried over anhydrous Na₂SO₄, the residue was concentrated under vacuum. The residues were purified by C₁₈ chromatography column eluted with ACN/H₂O (0.05% TFA) (v/v=1/5). This resulted in 0.110 g (55.32% yield) of 5-amino-6-methoxypicolinamide as brown solid. LCMS: m/z=168 [M+1]⁺.

### Step 2. 6-methoxy-5-(prop-2-yn-1-ylamino)picolinamide

Into a 8-mL three necked bottle purged and maintained with an inert atmosphere of nitrogen, was placed 5-amino-6-methoxypicolinamide (0.110 g, 658.03 μmol), dipotassium carbonate (0.653 g, 4.72 mmol), sodium iodide (0.136 g, 907.31 μmol), 3-bromopropyne (0.113 g, 949.90 μmol), N,N-dimethylformamide (2 mL). The mixture was stirred at 110° C. for 48 h. The resulting solution was quenched with water (5 mL) and extracted with EA (2×5 mL). The organic layer was combined and washed with brine (5 mL), dried over anhydrous Na₂SO₄, the residue was concentrated under vacuum. The residues were purified by C₁₈ chromatography column eluted with ACN/H₂O (0.05% TFA) (v/v=1/3). This resulted in 0.054 g (39.99% yields) of 6-methoxy-5-(prop-2-yn-1-ylamino)picolinamide as brown solid. LCMS: m/z=206 [M+1]⁺.

### Step 3. 5-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-6-methoxypicolinamide(racemic) (63)

Into a 8-mL sealed tube and maintained with an inert atmosphere of nitrogen, was placed 6-methoxy-5-(prop-2-yn-1-ylamino)picolinamide (0.055 g, 268.01 μmol), 3-methoxy-N-methyl-4-(prop-2-yn-1-ylamino)benzamide (0.110 g, 232.91 μmol), cuprous iodide (0.010 g, 52.51 μmol), [1,1′-bis(diphenylphosphino)ferrocene]dichloropalladium(II) (0.024 g, 34.00 μmol), triethylamine (0.054 g, 533.65 μmol), methyl sulfoxide (2 mL). The reaction mixture was stirred at 50 for 18 h. The reaction was quenched by the addition of water (2 mL) and extracted with EA (2×2 mL). The organic layer was combined, washed with brine (5 mL), dried over anhydrous Na₂SO₄ and concentrated under vacuum. The mixture was purified by preparative HPLC (Mobile Phase A: water (10 mmoL/L ammonium hydroxide), Mobile Phase B: MeOH; Flow rate: 70 mL/m in; Gradient: 55-80-100% B (2-30-60 min); 245 nm; RT: 32.600-33.523 min). This resulted in 4.42 mg (3.00% yield) of 5-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-6-methoxypicolinamide(racemic) (63) as white solid. LCMS: m/z=550 [M+1]⁺.

¹H NMR (400 MHz, DMSO) δ 7.65 (s, 1H), 7.56 (d, J=8.0 Hz, 1H), 7.26 (d, J=8.1 Hz, 1H), 7.20 d, J=8.3 Hz, 1H), 7.06 (d, J=7.9 Hz, 1H), 6.78 (d, J=7.5 Hz, 1H), 6.43 (s, 1H), 5.17 (s, 1H), 4.86 (s, 1H), 4.73 (s, 1H), 4.35 (d, J=6.5 Hz, 2H), 3.99 (s, 2H), 3.82 (d, J=11.6 Hz, 1H), 1.37 (d, J=6.8 Hz, 1H), 1.33 (s, 2H), 1.30 (s, 1H), 1.26 (s, 2H), 1.23 (s, 6H).

### Example 64

### (3-(difluoromethoxy)-4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)phenyl)dimethylphosphine oxide (racemic) (64)

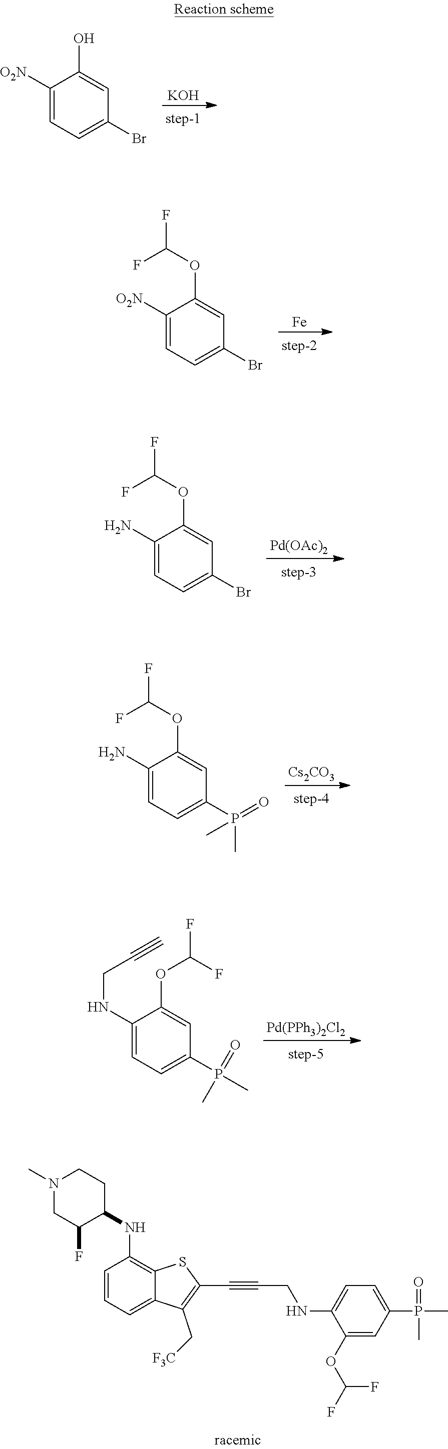

### Experimental Details

### Step 1. 4-bromo-2-(difluoromethoxy)-1-nitrobenzene

Into a 100-mL three necked bottle purged and maintained with an inert atmosphere of nitrogen, was placed 5-bromo-2-nitrophenol (7.20 g, 33.03 mmol), ((difluoromethyl)sulfonyl)benzene (11.01 g, 57.29 mmol), A CN (40 mL), KOH (1 M, 10 mL). The reaction mixture was stirred at 50° C. for 16 h. The resulting solution was added to water (100 mL). The resulting solution was extracted with EA (2×150 mL). The organic layers combined and washed with brine (100 mL), dried over anhydrous Na₂SO₄, the residue was concentrated under vacuum. The crude product purified by washing with HEX/EA (v/v=9/1). This resulted in 2.50 g (28.24% yield) of 4-bromo-2-(difluoromethoxy)-1-nitrobenzene as yellow oil. LCMS: m/z=268 [M+1]⁺

### Step 2. 4-bromo-2-(difluoromethoxy)aniline

Into a 50-mL flask was placed 4-bromo-2-(difluoromethoxy)-1-nitrobenzene (2.61 g, 9.74 mmol), Fe (2.36 g, 42.26 mmol), NH₄Cl (2.20 g, 41.13 mmol), MeOH (20 mL), H₂O (10 mL). The reaction mixture was stirred at 60° C. for 4 h. LCMS showed the reaction was completed. The resulting solution was added water (100 mL). The resulting solution was extracted with EA (2×150 mL). The organic layers combined and washed with brine (100 mL), dried over anhydrous Na₂SO₄, the residue was concentrated under vacuum. The crude product purified by washing with HEX/EA (v/v=9/1). This resulted in 1.897 g (81.84% yield) of 4-bromo-2-(difluoromethoxy)aniline as yellow oil. LCMS: m/z=238 [M+1]⁺.

### Step 3. (4-amino-3-(difluoromethoxy)phenyl)dimethylphosphine oxide

Into a 100-mL three necked bottle purged and maintained with an inert atmosphere of nitrogen, was placed 4-bromo-2-(difluoromethoxy)aniline (1.991 g, 8.36 mmol), Pd(OAc)₂ (1.026 g, 4.57 mmol), Xantphos (0.996 g, 1.72 mmol), DIEA (1.559 g, 12.06 mmol) and DMF (20 mL). The reaction mixture was stirred at 130° C. for 0.5 h. Then was added Dimethylphosphine oxide (2.065 g, 26.46 mmol). The reaction mixture w as stirred at 130° C. for another 1 h. LCMS showed that the reaction was completed. The reaction mixture was purified by C18 column eluted with ACN/H₂O (v/v=2/8). This resulted in 0.88 g (44.74% yield) of (4-amino-3-(difluoromethoxy)phenyl)dimethylphosphine oxide as red oil. LCMS: m/z=236 [M+1]⁺.

### Step 4. (3-(difluoromethoxy)-4-(prop-2-yn-1-ylamino)phenyl)dimethylphosphine oxide

Into a 50-mL flask was placed (4-amino-3-(difluoromethoxy)phenyl)dimethylphosphine (0.293 g, 1.24 mmol), 3-Bromopropyne (0.365 g, 3.07 mmol), K₂CO₃ (0.281 g, 2.03 mmol), KI (0.314 g, 1.89 mmol), DMF (5 mL). The reaction mixture was stirred at 80° C. for 4 h. LCMS showed that the reaction was completed. The reaction mixture was purified by C18 column eluted with ACN/H₂O (v/v=2/8). This resulted in 0.032 g (9.40% yield) of (3-(difluoromethoxy)-4-(prop-2-yn-1-ylamino)phenyl)dimethylphosphine oxide as red solid. LCMS: m/z=274 [M+1]⁺.

### Step 5. (3-(difluoromethoxy)-4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)phenyl)dimethylphosphine oxide (racemic) (64)

Into a 50-mL three necked bottle and maintained with an inert atmosphere of nitrogen, was placed (Z)-3-fluoro-N-(2-iodo-3-(2,2,2-trifluoroethyl)benzo[b]thiophen-7-yl)-1-methylpiperidin-4-amine (racemic) (0.064 g, 0.14 mmol), (3-(difluoromethoxy)-4-(prop-2-yn-1-ylamino)phenyl)dimethylphosphine oxide (0.066 g, 0.24 mmol), CuI (0.022 g, 0.12 mmol), Pd(PPh₃)₂Cl₂ (0.033 g, 0.047 mmol), DIEA (0.028 g, 0.22 mmol) and DMSO (5 mL). The reaction mixture was stirred at room temperature for 1 h. LCMS showed that the reaction was completed. The mixture was purified by preparative HPLC (Mobile Phase A: water (10 mmoL/L ammonium hydroxide), Mobile Phase B: ACN; Flow rate: 40 mL/min; Gradient: 40-75-75% B (2-30-60 min); 260 nm; RT: 30.053-31.242 min). This resulted in 0.002 g (2.39% yield) of (3-(difluoromethoxy)-4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)phenyl) dimethylphosphine oxide (racemic) (64) as off white solid. LCMS: m/z=618 [M+1]⁺

### Example 65

### (4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-(trifluoromethoxy)phenyl)dimethylphosphine oxide(racemic) (65)

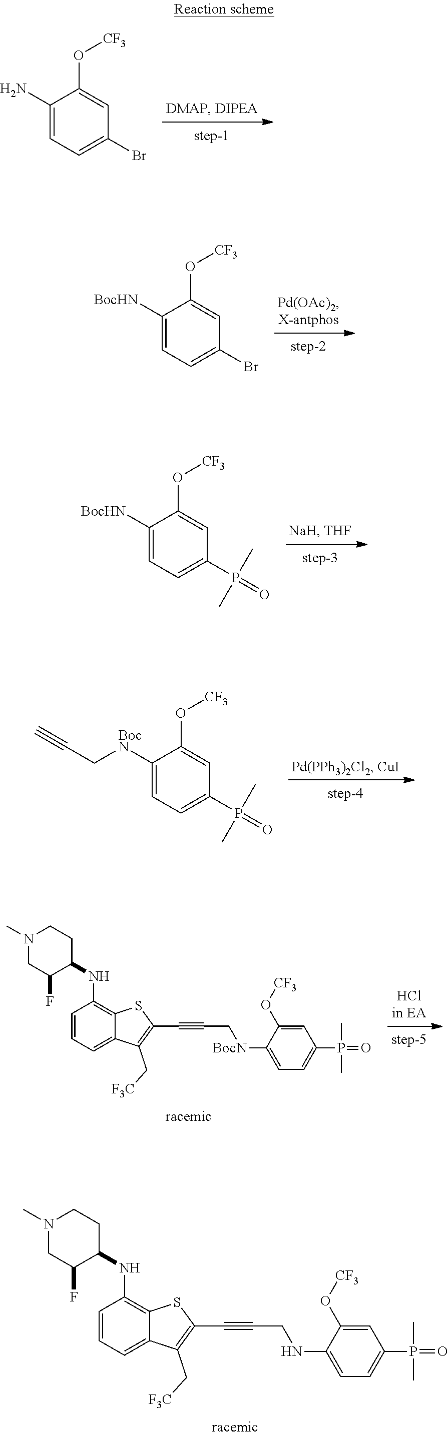 [SMILES: Nc1ccc(Br)cc1OC(F)(F)F ; CC(C)(C)OC(=O)Nc1ccc(Br)cc1OC(F)(F)F ; CC(C)(C)OC(=O)Nc1ccc(P(C)(C)=O)cc1OC(F)(F)F ; C#CCN(C(=O)OC(C)(C)C)c1ccc(P(C)(C)=O)cc1OC(F)(F)F ; CN1CC[C@@H](Nc2cccc3c(CC(F)(F)F)c(C#CCN(C(=O)OC(C)(C)C)c4ccc(P(C)(C)=O)cc4OC(F)(F)F)sc23)[C@@H](F)C1 ; CN1CC[C@@H](Nc2cccc3c(CC(F)(F)F)c(C#CCNc4ccc(P(C)(C)=O)cc4OC(F)(F)F)sc23)[C@@H](F)C1]

### Experimental Details

### Step 1. tert-butyl (4-bromo-2-(trifluoromethoxy)phenyl)carbamate

Into a 500-mL three necked bottle purged and maintained with an inert atmosphere of nitrogen, was placed 4-bromo-2-(trifluoromethoxy)aniline (10.02 g, 39.14 mmol), N,N-diisopropylethylamine (16.35 g, 126.51 m mol), N-(4-pyridyl)dimethylamine (0.20 g, 1.64 mmol), tetrahydrofuran (200 mL), di-tert-butyl dicarbonate (9.40 g, 43.07 mmol). The reaction mixture was stirred at 110° C. for 18 h. LCMS showed that the reaction was completed. The resulting solution was concentrated and purified by C₁₈ chromatography column elute d with ACN/H₂O (v/v=3/2). This resulted in 5.00 g (35.87% yield) of tert-butyl (4-bromo-2-(trifluoromethoxy)phenyl)carbamate as yellow solid. LCMS: m/z=357 [M+1]⁺.

### Step 2. tert-butyl (4-(dimethylphosphoryl)-2-(trifluoromethoxy)phenyl)carbamate

Into a 100-mL three necked bottle and maintained with an inert atmosphere of nitrogen, was placed tert-butyl (4-bromo-2-(trifluoromethoxy)phenyl)carbamate (1.99 g, 5.59 mmol), palladium (II) acetate (0.24 g, 1.07 mmol), dimethylbisdiphenylphosphinoxanthene (0.51 g, 881.41 μmol), N,N-diisopropylethylamine (1.61 g, 12.46 mmol), N,N-dimethylformamide (30 mL).

The reaction mixture was heated to 130° C. Then methylphosphinoylmethane (1.35 g, 17.30 mmol) was added and stirred at the same temperature for 1.0 h. LCMS showed that the reaction was completed. The residue was filtered and purified by C₁₈ chromatography column eluted with ACN/H₂O (0.05% TFA) (v/v=1/1). This resulted in 0.588 g (29.79% yield) of tert-butyl (4-(dimethylphosphoryl)-2-(trifluoromethoxy)phenyl) carbamate as yellow oil. LCMS: m/z=354 [M+1]⁺.

Step 3. tert-butyl (4-(dimethylphosphoryl)-2-(trifluoromethoxy)phenyl)(prop-2-yn-1-yl)carbamate Into a 25-mL three necked bottle purged and maintained with an inert atmosphere of nitrogen, was placed tert-butyl (4-(dimethylphosphoryl)-2-(trifluoromethoxy)phenyl)carbamate (0.58 g, 1.64 mmol), tetrahydrofuran (10 mL). The reaction mixture was stirred at 0° C. Then sodium hydride (0.27 g, 11.25 mmol) was added. The mixture was stirred at 20° C. for 1.0 h. Then 3-bromopropyne (0.30 g, 2.52 mmol) was added to the reaction at −10° C. The reaction mixture was stirred at 70° C. for 19 h. The resulting solution was added to water (10 mL). The resulting solution was extracted with EA (2×10 mL). The organic layer was combined and washed with brine (10 mL), dried over anhydrous Na₂SO₄, the residue was concentrated under vacuum. The residue was applied onto a silica gel column eluted with EA/hexane (v/v=1/3). This resulted in 0.35 g (54.52% yield) of tert-butyl (4-(dimethylphosphoryl)-2-(trifluoromethoxy)phenyl)(prop-2-yn-1-yl)carbamate as yellow oil. LCMS: m/z=392 [M+1]⁺.

### Step 4. tert-butyl (7-(dimethylphosphoryl)benzo[d][1,3]dioxol-4-yl)(prop-2-yn-1-yl)carbamate(racemic)

Into a 8-mL three necked bottle and maintained with an inert atmosphere of nitrogen, was placed cuprous iodide (0.010 g, 52.51 μmol), triethylamine (0.138 g, 1.36 mmol), (Z)-3-fluoro-N-(2-iodo-3-(2,2,2-trifluoroethyl)benzo[b]thiophen-7-yl)-1-methylpiperidin-4-amine (0.202 g, 427.71 μmol), [1,1′-bis(diphenylphosphino)ferrocene]dichloropalladium(II) (0.063 g, 89.24 μmol), Methyl sulfoxide (2 mL). The reaction mixture was stirred at 80° C. for 10 min. Then tert-butyl (4-(dimethylphosphoryl)-2-(trifluoromethoxy)phenyl)(prop-2-yn-1-yl)carbamate (0.21 g, 536.64 μmol) was added and stirred at the same temperature for 1.5 h. LCMS showed that the reaction was completed. The resulting solution was quenched with water (10 mL) and extracted with EA (2×10 mL). The organic layer was combined and washed with brine (10 mL), dried over anhydrous Na₂SO₄, the residue was concentrated under vacuum. The residue was purified by C₁₈ chromatography column eluted with ACN/H₂O (v/v=2/5). This resulted in 0.190 g (60.38% yield) of tert-butyl (4-(dimethylphosphoryl)-2-(trifluoromethoxy)phenyl)(3-(7-(((Z)-3-fluoro-1-methylpiperidin-4-yl)amino)-3-(2,2,2-trifluoroethyl)benzo[b]thiophen-2-yl)prop-2-yn-1-yl)carbamate(racemic) as yellow solid. LCMS: m/z=736 [M+1]⁺.

### Step 5. (4-((3-(7-(((Z)-3-fluoro-1-methylpiperidin-4-yl)amino)-3-(2,2,2-trifluoroethyl)benzo[b]thiophen-2-yl) prop-2-yn-1-yl)amino)-3-(trifluoromethoxy)phenyl)dimethylphosphine oxide(racemic) (65)

Into a 25-mL round-bottom flask purged and maintained with an inert atmosphere of nitrogen, was placed tert-butyl (4-(dimethylphosphoryl)-2-(trifluoromethoxy)phenyl)(3-(7-(((Z)-3-fluoro-1-methylpiperidin-4-yl)amino)-3-(2,2,2-trifluoroethyl)benzo[b]thiophen-2-yl)prop-2-yn-1-yl)carbamate (0.189 g, 256.90 μmol), ethyl acetate (1 mL) and HCl in EA solution (5 mL, 4 M). The reaction mixture was stirred at room temperature for 0.5 h. LCMS showed that the reaction was completed. Then saturated sodium hydrogen carbonate aqueous solution was added to the mixture until pH=7˜8. The resulting solution was extracted with EA (2×5 mL). The organic layer was combined, washed with brine (5 mL), dried over anhydrous Na₂SO₄ and concentrated under vacuum. The mixture was purified by preparative HPLC (Mobile Phase A: water (10 mmoL/L ammonium hydroxide), Mobile Phase B: ACN; Flow rate: 70 mL/min; Gradient: 35-70-95% B (2-30-60 min; 260 nm; RT: 27.958-33.448 min). This resulted in 0.088 g (53.90% yield) of (4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-(trifluoromethoxy)phenyl)dimethylphosphine oxide(racemic) (65) as white solid. LCMS: m/z=636 [M+1]⁺.

¹H NMR (400 MHz, DMSO) δ 7.63-7.50 (m, 2H), 7.27 (t, J=7.7 Hz, 1H), 7.21 (d, J=7.9 Hz, 1H), 7.13-7.07 (m, 1H), 6.83 (t, J=6.4 Hz, 1H), 6.78 (d, J=7.7 Hz, 1H), 5.19 (d, J=8.3 Hz, 1H), 4.82 (d, J=49.3 Hz, 1H), 4.39 (d, J=6.2 Hz, 2H), 3.87-3.71 (m, 2H), 3.63 (s, 1H), 3.08 (s, 1H), 2.83 (s, 1H), 2.33 (s, 1H), 2.22 (s, 3H), 2.17-2.07 (m, 1H), 1.96 (d, J=9.8 Hz, 1H), 1.73 (d, J=10.7 Hz, 1H), 1.61 (s, 3H), 1.58 (s, 3H).

### Example 66

### diethyl(4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-(fluoromethoxy)phenyl)phosphine oxide (66)

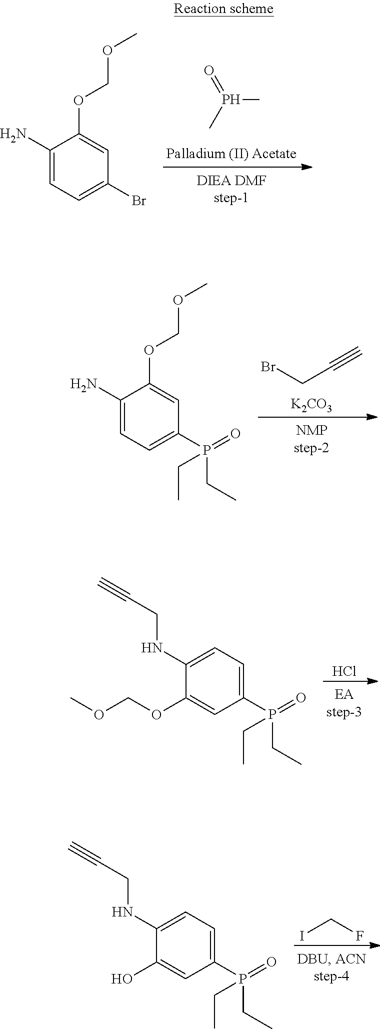

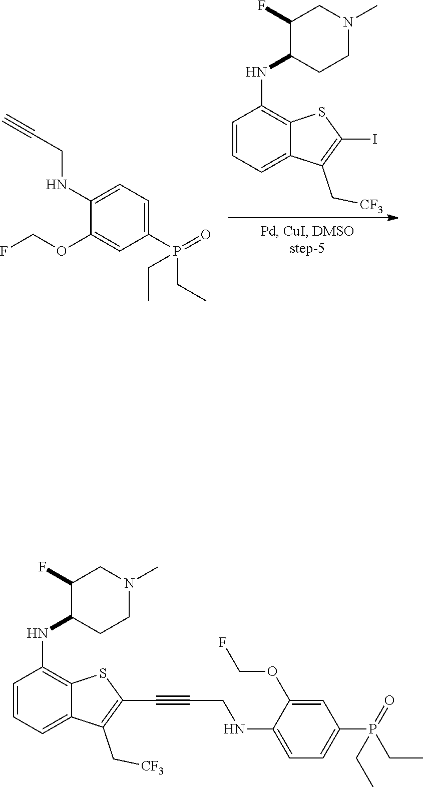 [SMILES: C#CCNc1ccc(P(=O)(CC)CC)cc1OCF ; CN1CC[C@@H](Nc2cccc3c(CC(F)(F)F)c(I)sc23)[C@@H](F)C1 ; CCP(=O)(CC)c1ccc(NCC#Cc2sc3c(N[C@@H]4CCN(C)C[C@@H]4F)cccc3c2CC(F)(F)F)c(OCF)c1]

### Experimental Details

### Step 1. (4-amino-3-(methoxymethoxy)phenyl)diethylphosphine oxide

Into a 100-mL three necked bottle purged and maintained with an inert atmosphere of nitrogen, was placed 4-bromo-2-(methoxymethoxy)aniline (2.576 g, 11.09 mmol), Palladium (II) Acetate and (0.514 g, 2.28 mmol), Xantphos (1.058 g, 1.82 mmol), DIEA (4.957 g, 3.35 mmol) and DMF (30 mL). The reaction mixture was stirred at 130° C. Then dimethylphosphine oxide (2.004 g, 18.88 mmol) was added. The mixture was stirred at 130° C. for 1 h. The reaction mixture was filtered through celite pad and celite pad was washed with ACN (3×20 mL). The filterate was then concentrated under reduced pressure to afford the crude product. The crude product was purified by C18 column eluted with ACN/H₂O (v/v=1/4), filtered and concentrated under vacuum. This resulted in 0.89 g (31.66% yield) of (4-amino-3-(methoxymethoxy)phenyl)diethylphosphine oxide as yellow oil. LCMS: m/z=256 [M+1]⁺.

### Step 2. diethyl(3-(methoxymethoxy)-4-(prop-2-yn-1-ylamino)phenyl)phosphine oxide

Into a 25 ml 3-necked flask was placed (4-amino-3-(methoxymethoxy)phenyl)diethylphosphine oxide (0.721 g, 2.80 mmol), 3-bromoprop-1-yne (0.552 g, 4.64 mmol), K₂CO₃ (0.798 g, 5.77 mmol), KI (0.614 g, 3.69 mmol) DMF (10 mL). The reaction mixture was stirred under nitrogen at 80° C. for 4 h. The reaction mixture was filtered through celite pad and celite pad was washed with ACN (3×20 mL). The filterate was then concentrated under reduced pressure to afford the crude product. The crude product was purified by C18 column eluted with ACN/H₂O (v/v=1/3), filtered and concentrated under vacuum. This resulted in 0.429 g (51.83% yield) of diethyl(3-(methoxymethoxy)-4-(prop-2-yn-1-ylamino)phenyl)phosphine oxide as brown oil. LCMS: m/z=296 [M+1]⁺.

### Step 3. diethyl(3-hydroxy-4-(prop-2-yn-1-ylamino)phenyl)phosphine oxide

Into a 25-mL round-bottom flask purged and maintained with an inert atmosphere of nitrogen, was placed diethyl(3-(methoxymethoxy)-4-(prop-2-yn-1-ylamino)phenyl)phosphine oxide (0.394 g, 1.33 mmol), ethyl acetate (4 mL) and HCl in EA solution (4 mL, 4 M). The reaction mixture was stirred at room temperature for 3 h. The mixture was concentrated to afford the crude product. This resulted in 0.337 g (100.52% yield) of diethyl(3-hydroxy-4-(prop-2-yn-1-ylamino)phenyl)phosphine oxide as brown oil. LCMS: m/z=252 [M+1]⁺.

### Step 4. diethyl(3-(fluoromethoxy)-4-(prop-2-yn-1-ylamino)phenyl)phosphine oxide

Into a 25-mL round-bottom flask purged and maintained with an inert atmosphere of nitrogen, was placed diethyl(3-hydroxy-4-(prop-2-yn-1-ylamino)phenyl)phosphine oxide (0.323 g, 1.28 mmol), fluoro-iodo-methane (0.414 g, 2.58 mmol), ACN (4 mL). The reaction mixture was stirred at room temperature for 16 h. The reaction mixture was filtered through celite pad and celite pad was washed with ACN (3×20 mL). The filterate was then concentrated under reduced pressure to afford the crude product. The reaction mixture was purified by C18 column eluted with ACN/H₂O (v/v=1/3), filtered and concentrated under vacuum. This resulted in 0.091 g, (24.98% yield) of diethyl(3-(fluoromethoxy)-4-(prop-2-yn-1-ylamino)phenyl)phosphine oxide as brown oil. LCMS: m/z=284 [M+1]⁺.

### Step 5. diethyl(4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-(fluoromethoxy)phenyl)phosphine oxide (66)

Into a 10-mL round-bottom flask was placed diethyl(3-(fluoromethoxy)-4-(prop-2-yn-1-ylamino)phenyl)phosphine oxide (0.090 g, 317.70 umol), (Z)-3-fluoro-N-(2-iodo-3-(2,2,2-trifluoroethyl)benzo[b]thiophen-7-yl)-1-meth ylpiperidin-4-amine (0.083 g, 175.74 umol), Pd(PPh₃)₂Cl₂ (0.023 g, 32.58 μmol), CuI (0.019 g, 99.76 umol), DIEA (0.097 g, 750.52 umol), methyl sulfoxide (2 mL). The reaction was stirred under nitrogen atmosphere at RT for 3 h. The reaction mixture was filtered through celite pad and celite pad was washed with ACN (3×20 mL). The filterate was then concentrated under reduced pressure to afford the crude product. The crude product was purified by preparative HPLC (Mobile Phase A: water (0.1% ammonium hydroxide), Mobile Phase B: ACN; Flow rate: 40 mL/min; Gradient: 40-70-90% B (2-30-60 min); 254 nm; RT: 37.586-38.778) to provide the desired product. This resulted in 0.020 g (10.98% yield) of diethyl(4-((3-(7-(((Z)-3-fluoro-1-m ethylpiperidin-4-yl)amino)-3-(2,2,2-trifluoroethyl)benzo[b]thiophen-2-yl)prop-2-yn-1-yl)amino)-3-(fluoromethoxy) phenyl)phosphine oxide (66) as white solid. LCMS: m/z=628 [M+1]⁺.

¹H NMR (400 MHz, DMSO-d₆) δ 7.36-7.19 (m, 4H), 6.96-6.94 (m, 1H), 6.79 (d, J=7.6 Hz, 1H), 6.33 (t, J=6.3 Hz, 1H), 5.91 (s, 1H), 5.77 (s, 1H), 5.12 (d, J=8.5 Hz, 1H), 4.80 (d, J=49.4 Hz, 1H), 4.37 (d, J=6.2 Hz, 2H), 3.82-3.78 (m, 2H), 3.03 (t, J=11.5 Hz, 1H), 2.80 (d, J=11.2 Hz, 1H), 2.31-2.05 (m, 5H), 1.99-1.71 (m, 7H), 0.98-0.94 (m, 6H).

### Example 67

### (4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)(piperidin-1-yl)methanone(racemic) (67)

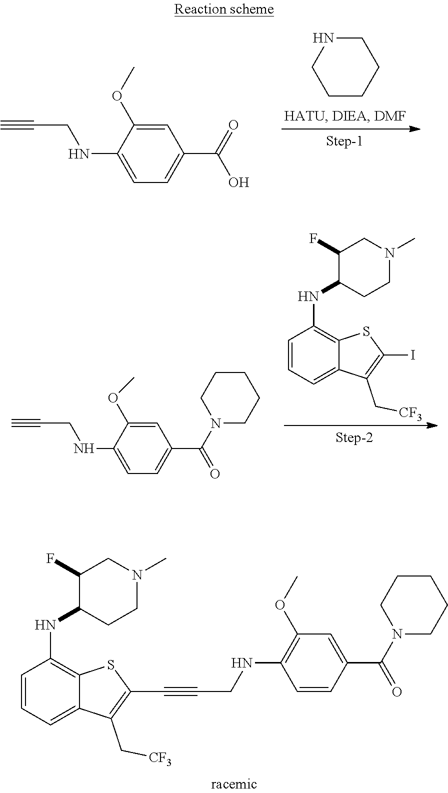

### Step 1. (3-methoxy-4-(prop-2-yn-1-ylamino)phenyl)(piperidin-1-yl)methanone

Into a 50-mL three necked bottle purged and maintained with an inert atmosphere of nitrogen, was placed 3-methoxy-4-(prop-2-yn-1-ylamino)benzoic acid (102.00 mg, 0.50 mmol), HATU (296.00 mg, 0.79 mmol), DIEA (251.00 mg, 1.94 mmol), DMF (3 mL). The reaction mixture was stirred at room temperature for 6 h. The residue was applied onto a C₁₈ column eluted with ACN:H₂O (0.01% TFA) (v:v=1:1), Flow rate: 80 mL/min. This resulted in 129.00 mg (95.29% yield) of (3-methoxy-4-(prop-2-yn-1-ylamino)phenyl)(piperidin-1-yl)methanone as brown solid. LCMS: m/z=273 [M+1]⁺

### Step 2. (4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)(piperidin-1-yl)methanone(racemic) (67)

Into a 8-mL reaction vial purge and maintained with an inert atmosphere of nitrogen, was placed (Z)-3-fluoro-N-(2-iodo-3-(2,2,2-trifluoroethyl)benzo[b]thiophen-7-yl)-1-methylpiperidin-4-amine (99.00 mg, 0.21 mmol), (3-methoxy-4-(prop-2-yn-1-ylamino)phenyl)(piperidin-1-yl)methanone (129.00 mg, 0.47 mmol), CuI (21 mg, 0.11 mmol), Pd(PPh₃)₂Cl₂ (23.00 mg, 0.032 mmol), DIEA (126.00 mg, 0.97 mmol), DMSO (3 mL). The reaction mixture was stirred at room temperature for 6 h. The residue was applied onto a C₁₈ column eluted with ACN:H₂O (0.01% TFA) (v:v=1:1), Flow rate: 80 mL/min. This resulted in 41.00 mg (31.71% yield) of (4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)(piperidin-1-yl)methanone(racemic) (67) as yellow solid. LCMS: m/z=617 [M+1]⁺

¹H NMR (400 MHz, DMSO) δ 7.36-7.16 (m, 2H), 6.98-6.83 (m, 2H), 6.80-6.73 (m, 2H), 5.92 (t, J=6.3 Hz, 1H), 5.16 (d, J=8.5 Hz, 1H), 4.86-4.73 (m, 1H), 4.33 (d, J=6.3 Hz, 2H), 3.89-3.74 (m, 5H), 3.73-3.57 (m, 1H), 3.46 (s, 4H), 3.06-3.01 (m, 1H), 2.80 (d, J=10.7 Hz, 1H), 2.32-2.14 (m, 4H), 2.13-2.02 (m, 1H), 2.00-1.90 (m, 1H), 1.77-1.70 (m, 1H), 1.66-1.61 (m, 2H), 1.53-1.45 (m, 4H).

### Example 68

### N-(3-fluoro-1-methylpiperidin-4-yl)-4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-methoxybenzamide (68)

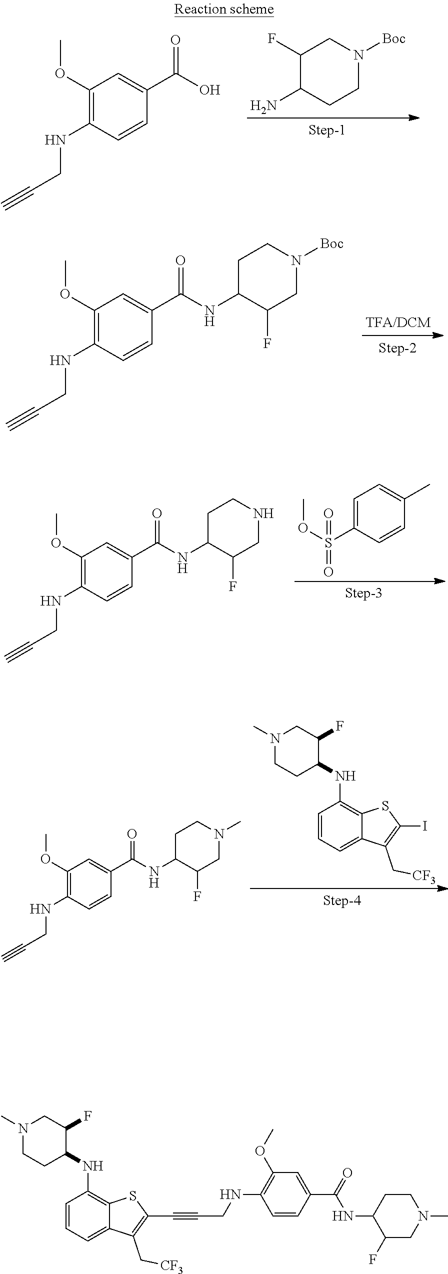

### Experiment Details

### Step 1. tert-butyl 3-fluoro-4-(3-methoxy-4-(prop-2-yn-1-ylamino)benzamido)piperidine-1-carboxylate

Into a 8 mL vail was placed 3-methoxy-4-(prop-2-yn-1-ylamino)benzoic acid (0.195 g, 950.25 μmol), tert-butyl 4-amino-3-fluoropiperidine-1-carboxylate (0.260 g, 1.19 mmol), HATU (0.420 g, 1.10 mmol), MeCN (3 mL). The reaction was stirred overnight at room temperature. LCMS showed the reaction was complete. The reaction was quenched with water (10 mL), extracted with EA (4 mL×2). The combined organic layers were washed with water (10 mL) and brine (10 mL) successively, separated, then concentrated with vacuum to afford crude tert-butyl 3-fluoro-4-(3-methoxy-4-(prop-2-yn-1-ylamino)benzamido)piperidine-1-carboxylate (0.422 g, 1.04 mmol, 109.53% yield) as off-white solid. LCMS: m/z=406[M+1]⁺.

### Step 2. N-(3-fluoropiperidin-4-yl)-3-methoxy-4-(prop-2-yn-1-ylamino)benzamide

Into a 50 mL flask was placed tert-butyl 3-fluoro-4-(3-methoxy-4-(prop-2-yn-1-ylamino)benzamido)piperidine-1-carboxylate (0.420 g, 1.04 mmol), TFA (1 mL), DCM (5 mL). The reaction was stirred overnight at room temperature. LCMS showed the reaction was complete. The reaction quenched with saturated NaHCO₃ aq., The solution was diluted with water (10 mL), extracted with EA (5 mL×3). The combined organic layers were washed with water (5 mL) and brine (5 mL) successively, separated, then concentrated with vacuum. The residue was purified with C₁₈ column, eluted with ACN/water (v/v=1/3) to afford N-(3-fluoro piperidin-4-yl)-3-methoxy-4-(prop-2-yn-1-ylamino)benzamide (0.200 g, 654.99 μmol, 63.23% yield) as an off-white solid. LCMS: m/z=306[M+1]⁺

### Step 3. N-(3-fluoro-1-methylpiperidin-4-yl)-3-methoxy-4-(prop-2-yn-1-ylamino)benzamide

Into a 4 mL vail was placed N-(3-fluoropiperidin-4-yl)-3-methoxy-4-(prop-2-yn-1-ylamino)benzamide (0.191 g, 625.52 μmol), methyl 4-methylbenzenesulfonate (196.3056 mg, 1.05 mmol), K₂CO₃ (191.0000 mg, 1.38 mmol), THF (1 mL). The reaction was stirred for 1.5 h at 0° C. LCMS showed the reaction was complete. The residue was purified with Silica-gel column, eluted with MeOH/DCM (v/v=1/10) to afford N-(3-fluoro-1-methylpiperidin-4-yl)-3-methoxy-4-(prop-2-yn-1-ylamino)benzamide (0.108 g, 338.16 μmol, 54.06% yield) as an light yellow oil. LCMS: m/z=320[M−1]⁻

### Step 4. N-(3-fluoro-1-methylpiperidin-4-yl)-4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-methoxybenzamide (68)

Into a 4 mL vial purged and maintained with nitrogen atmosphere was placed (Z)-3-fluoro-N-(2-iodo-3-(2,2,2-trifluoroethyl)benzo[b]thiophen-7-yl)-1-methylpiperidin-4-amine (0.183 g, 387.4808 μmol), N-(3-fluoro-1-methylpiperidin-4-yl)-3-methoxy-4-(prop-2-yn-1-ylamino)benzamide (0.103 g, 322.51 μmol), Pd(PPh₃)₂Cl₂ (2.451 g, 2.68 μmol), CuI (0.010 g, 52.51 μmol), TEA (0.044 g, 434.83 μmol), DMF (1 mL), and stirred for 3 h at room temperature. LCMS monitored that the reaction was complete. The reaction was quenched with water (4 mL), extracted with EA (3 mL×2). The combined organic layers were washed with water (4 m L) and brine (4 mL) successively, separated, then concentrated with vacuum. The residue was purified with prep-HPLC (Mobile Phase A: water (formic acid), Mobile Phase B: ACN; Flow rate: 70 mL/min; Gradient: 25-45-45% B (2-30-60 min); 262 nm; RT: 27.501-29.585 min) to afford N-(3-fluoro-1-methylpiperidin-4-yl)-4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-methoxybenzamide (68) (0.010 g, 15.07 μmol, 3.89% yield) as a white solid. LCMS: m/z=665 [M+1]⁺.

¹H NMR (400 MHz, DMSO-d₆) δ 8.08 (dd, J=32.3, 7.9 Hz, 1H), 7.56-7.45 (m, 1H), 7.38 (dd, J=23.3, 1.9 Hz, 1H), 7.32-7.18 (m, 2H), 6.83-6.72 (m, 2H), 6.07 (t, J=6.4 Hz, 1H), 5.17 (d, J=8.5 Hz, 1H), 4.88-4.71 (m, 1H), 4.70-4.47 (m, 1H), 4.35 (d, J=6.3 Hz, 2H), 4.00-3.57 (m, 7H), 3.17-3.00 (m, 2H), 2.84-2.64 (m, 2H), 2.20 (d, J=21.4 Hz, 7H), 2.04-1.91 (m, 3H), 1.87-1.46 (m, 4H).

### Example 69

### 4-((3-(7-((3-fluoro-1-methylpiperidin-4-yl)amino)-3-(2,2,2-trifluoroethyl)benzo[b]thiophen-2-yl)prop-2-yn-1-yl)amino)-3-methoxy-N-(2-methyltetrahydro-2H-pyran-4-yl)benzamide (69)

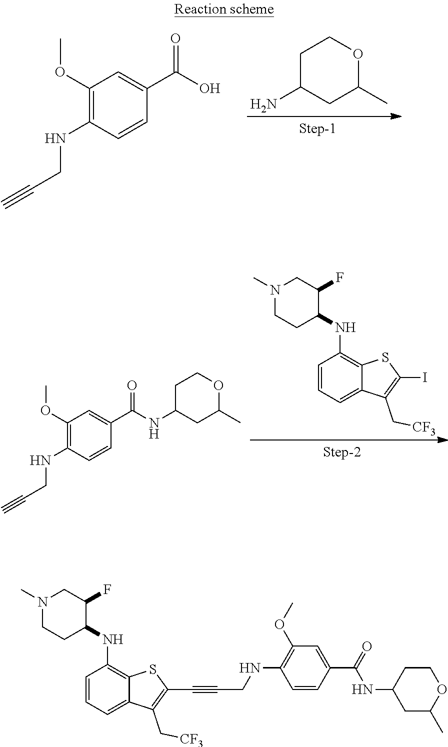

### Experiment Details

### Step 1. 3-methoxy-N-(2-methyltetrahydro-2H-pyran-4-yl)-4-(prop-2-yn-1-ylamino)benzamide

Into a 8 mL vail was placed 3-methoxy-4-(prop-2-yn-1-ylamino)benzoic acid (0.098 g, 477.56 μmol), 2-methyltetrahydro-2H-pyran-4-amine (0.206 g, 2.04 mmol), HATU (0.235 g, 618.05 μmol), MeCN (1 mL). The reaction was stirred overnight at room temperature. LCMS showed the reaction was complete. The reaction was quenched with water (10 mL), extracted with EA (4 mL×2). The combined organic layers were washed with water (10 mL) and brine (10 mL) successively, separated, then concentrated with vacuum to afford crude 3-methoxy-N-(2-methyltetrahydro-2H-pyran-4-yl)-4-(prop-2-yn-1-ylamino)benzamide (0.192 g, 665.88 μmol, 139.43% yield) as off-white solid. LCMS: m/z=303[M+1]⁺.

### Step 2. 4-((3-(7-((3-fluoro-1-methylpiperidin-4-yl)amino)-3-(2,2,2-trifluoroethyl)benzo[b]thiophen-2-yl)prop-2-yn-1-yl)amino)-3-methoxy-N-(2-methyltetrahydro-2H-pyran-4-yl)benzamide (69)

Into a 4 mL vial purged and maintained with nitrogen atmosphere was placed (Z)-3-fluoro-N-(2-iodo-3-(2,2,2-trifluoroethyl)benzo[b]thiophen-7-yl)-1-methylpiperidin-4-amine (0.103 g, 218.09 μmol), 3-methoxy-N-(2-methyltetrahydro-2H-pyran-4-yl)-4-(prop-2-yn-1-ylamino)benzamide (0.115 g, 398.83 μmol), Pd(PPh₃)₂Cl₂ (0.019 g, 26.91 μmol), CuI (0.007 g, 36.76 μmol), TEA (0.045 g, 444.71 μmol), DMF (1 mL), and stirred for 3 h at room temperature. LCMS monitored that the reaction was complete. The reaction was quenched with water (4 mL), extracted with EA (3 mL×2). The combined organic layers were washed with water (4 m L) and brine (4 mL) successively, separated, then concentrated with vacuum. The residue was pre-purified with Silica-gel column (DCM/MeOH v/v=1/10), then re-purified with C₁₈ column (ACN/water v/v=1/5) to afford N-(3-fluoro-1-methylpiperidin-4-yl)-4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-methoxybenzamide (69) (0.123 g, 194.40 μmol, 89.14% yield). LCMS: m/z=647 [M+1]⁺.

¹H NMR (400 MHz, DMSO-d₆) δ 7.32-7.18 (m, 2H), 6.97-6.89 (m, 2H), 6.82-6.73 (m, 2H), 5.97 (t, J=6.4 Hz, 1H), 5.15 (d, J=8.5 Hz, 1H), 4.79 (d, J=49.8 Hz, 1H), 4.33 (d, J=6.3 Hz, 2H), 3.81 (d, J=9.0 Hz, 9H), 3.53-3.40 (m, 2H), 3.03 (t, J=11.3 Hz, 2H), 2.82-2.65 (m, 2H), 2.27 (d, J=13.0 Hz, 1H), 2.18 (s, 3H), 2.10-1.91 (m, 2H), 1.71 (d, J=12.6 Hz, 1H), 1.06 (d, J=6.1 Hz, 3H).

### Example 70

### (4-((3-(7-((((2R,7aS)-2-fluorotetrahydro-1H-pyrrolizin-7a(5H)-yl)methyl)amino)-3-(2,2,2-trifluoroethyl)benzo[b]thiophen-2-yl)prop-2-yn-1-yl)amino)-3-methoxyphenyl)dimethylphosphine oxide (70)

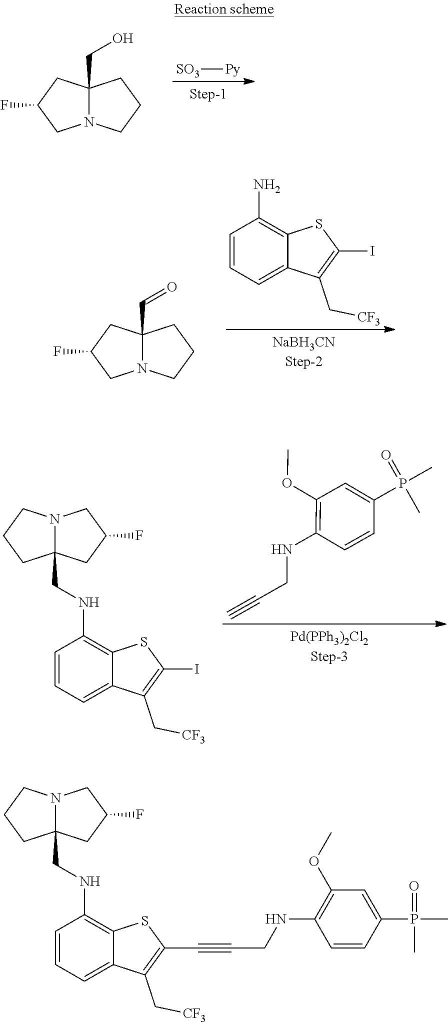

### Experiment Details

### Step 1. (2R,7aS)-2-fluorotetrahydro-1H-pyrrolizine-7a(5H)-carbaldehyde

Into a 4 mL vail was placed ((2R,7aS)-2-fluorotetrahydro-1H-pyrrolizin-7a(5H)-yl)methanol (0.502 g, 3.15 m mol), TEA (3.035 g, 29.99 mmol), DMSO (20 mL), DCM (10 mL). The reaction was cooled to 09.99 m mol), DMSO (20 mL),), 5.9 (2.066 g, 12.98 mmol) was added. The reaction was stirred overnight at room temperature. LCMS showed the reaction was complete. The reaction was quenched with water (50 mL), extracted with EA (20 mL×2). The aqueous layer was concentrated with vacuum to afford crude (2R,7a S)-2-fluorotetrahydro-1H-pyrrolizine-7a(5H)-carbaldehyde (0.340 g, 2.16 mmol, 68.60% yield) as white solid. LCMS: m/z=158[M+1]⁺.

### Step 2. N-(((2R,7aS)-2-fluorotetrahydro-1H-pyrrolizin-7a(5H)-yl)methyl)-2-iodo-3-(2,2,2-trifluoroethyl)benzo[b]thiophen-7-amine

Into a 4 mL flask was placed tetrahydro-1H-pyrrolizine-7a(5H)-carbaldehyde (0.330 g, 2.10 mmol), 2-iodo-3-(2,2,2-trifluoroethyl)benzo[b]thiophen-7-amine (0.154 g, 431.21 i mol). The reaction was heated to 150eactio stirred for 20 min then cooled to room temperature naturally. Sodium cyanoborohydride (0.244 g, 3.95 m mol), acetic acid (0.01 μmol), ethanol (1 mL) was added and stirred for 1 h at room temperature. LCMS monitored the reaction was complete. the reaction was purified with Silica-gel column (PE/EA v/v=3/1) to afford N-(((2R,7aS)-2-fluorotetrahydro-1H-pyrrolizin-7a(5H)-yl)methyl)-2-iodo-3-(2,2,2-trifluoroethyl)benzo[b]thiophen-7-amine (0.066 g, 132.45 μmol, 6.301% yield) as off-white solid. LCMS: m/z=481[M+1]⁺.

### Step 3. (4-((3-(7-((((2R,7aS)-2-fluorotetrahydro-1H-pyrrolizin-7a(5H)-yl)methyl)amino)-3-(2,2,2-trifluoroethyl)benzo[b]thiophen-2-yl)prop-2-yn-1-yl)amino)-3-methoxyphenyl)dimethylphosphine oxide (70)

Into a 4 mL vial purged and maintained with nitrogen atmosphere was placed 3-fluoro-(3-methoxy-4-(prop-2-yn-1-ylamino)phenyl)dimethylphosphine oxide (0.052 g, 219.19 μmol), N-(3-fluoro-1-methylpiperidin-4-yl)-3-methoxy-4-(prop-2-yn-1-ylamino)benzamide (0.057 g, 114.38 μmol), Pd(PPh₃)₂Cl₂ (0.011 g, 15.58 μmol), CuI (0.002 g, 10.50 μmol), TEA (0.030 g, 296.47 μmol), DMF (1 mL), and stirred for 2.5 h at room temperature. LCMS monitored that the reaction was complete. The reaction was quenched with water (2 mL), extracted with EA (2 mL×2). The combined organic layers were washed with water (2 mL) and brine (2 mL) successively, separated, then concentrated with vacuum. The residue was pre-purified with prep-TL C, gradient with DCM/MeOH (v/v=1/5), then re-purified with C₁₈-column, eluted with ACN/water (v/v=1/3), to afford (4-((3-(7-((((2R,7aS)-2-fluorotetrahydro-1H-pyrrolizin-7a(5H)-yl)methyl)amino)-3-(2,2,2-trifluoro ethyl)benzo[b]thiophen-2-yl)prop-2-yn-1-yl)amino)-3-methoxyphenyl)dimethylphosphine oxide (70) (0.019 g, 31.27 μmol, 27.34% yield) as a white solid. LCMS: m/z=608 [M+1]⁺.

¹H NMR (400 MHz, DMSO-d₆) δ 7.29-7.12 (m, 4H), 6.87-6.81 (m, 1H), 6.77 (d, J=7.8 Hz, 1H), 6.06 (t, J=6.3 Hz, 1H), 5.33-5.15 (m, 1H), 4.93 (t, J=5.9 Hz, 1H), 4.34 (d, J=6.4 Hz, 2H), 3.82 (d, J=28.4 Hz, 5H), 3.14-3.10 (m, 2H), 3.07-2.99 (m, 2H), 2.20-1.66 (m, 8H), 1.58 (d, J=13.1 Hz, 6H).

### Example 71

### (3-methoxy-4-((3-(7-((1-methylpiperidin-4-yl)amino)-3-(2,2,2-trifluoroethyl)thieno[3,2-b]pyridin-2-yl)prop-2-yn-1-yl)amino)phenyl)dimethylphosphine oxide (71)

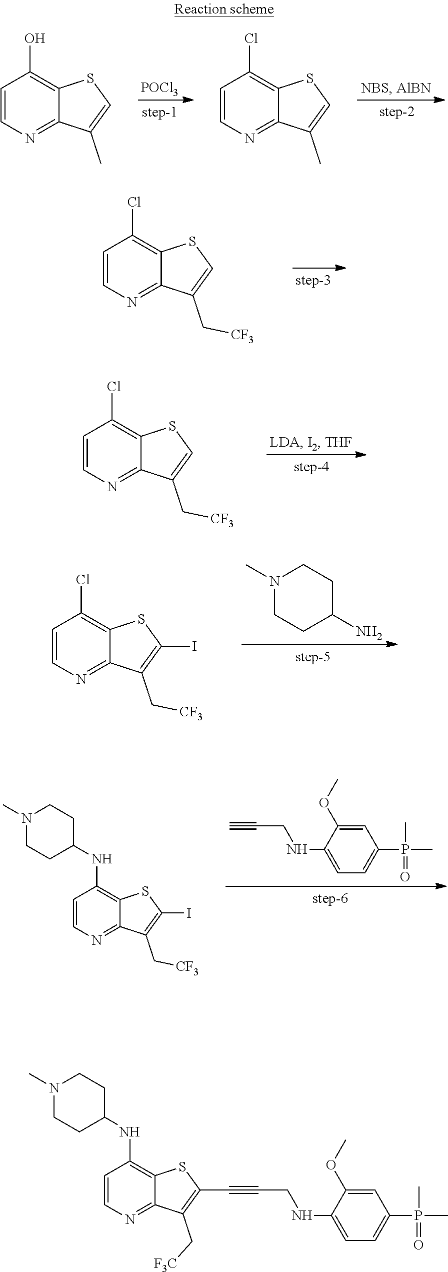

### Step 1. 7-chloro-3-methylthieno[3,2-b]pyridine

Into a 100-mL three necked bottle purged and maintained with an inert atmosphere of nitrogen, was placed 3-methylthieno[3,2-b]pyridin-7-ol (1.00 g, 6.07 mmol), POCl₃ (6 mL). The reaction mixture was stirred at 110° C. for 1.5 h. The reaction mixture was cooled to room temperature and POCl₃ was concentrated under vacuum. The resulting solution was added to water (50 mL). The resulting solution was extracted with EA (2×50 mL). The organic layers combined and washed with brine (20 mL), dried over anhydrous Na₂SO₄, the residue was concentrated under vacuum. The residue was applied onto a silica gel column eluted with EA:hexane (v:v=1:1). This resulted in 1.10 g (98.55% yield) of 7-chloro-3-methylthieno[3,2-b]pyridine as yellow solid. LCMS: m/z=183 [M+1]⁺

### Step 2. 3-(bromomethyl)-7-chlorothieno[3,2-b]pyridine

Into a 100-mL three necked bottle purged and maintained with an inert atmosphere of nitrogen, was placed 7-chloro-3-methylthieno[3,2-b]pyridine (1.80 g, 9.80 mmol), NBS (2.50 g, 14.04 mmol), AIBN (200.00 mg, 1.21 mmol), EA (20 mL). The mixture was stirred at 80° C. for 5 h. The resulting solution was cooled to room temperature and quenched with water (100 mL). The resulting solution was extracted with EA (2×100 mL). The organic layers combined and washed with brine (50 mL), dried over anhydrous Na₂SO₄, the residue was concentrated under vacuum. The residue was applied onto a silica gel column eluted with EA:hexane (v:v=1:1). This resulted in 1.50 g (58.29% yield) of 3-(bromomethyl)-7-chlorothieno[3,2-b]pyri dine as yellow solid. LCMS: m/z=262 [M+1]⁺.

### Step 3. 7-chloro-3-(2,2,2-trifluoroethyl)thieno[3,2-b]pyridine

Into a 100-mL three necked bottle purged and maintained with an inert atmosphere of nitrogen, was placed 3-(bromomethyl)-7-chlorothieno[3,2-b]pyridine (1.01 g, 3.84 mmol), diphenyl-(trifluoromethyl)-sulfonium trifluoromethanesulfonate (3.62 g, 8.95 mmol), Cu (0.71 g, 11.17 mmol), NMP (20 mL). The reaction mixture was stirred at 60° C. for 4 h. The resulting solution was cooled to room temperature and quenched with H₂O (100 mL). The resulting solution was filtered through a celite pad, and the filtrate extracted with EA (2×100 mL). The organic layers combined and washed with brine (50 mL), dried over anhydrous Na₂SO₄, the residue was concentrated under vacuum. The residue was applied onto a silica gel column eluted with EA/hexane (v/v=1/10). This resulted in 800.00 mg (82.63% yield) of 7-chloro-3-(2,2,2-trifluoroethyl)thieno[3,2-b]pyridine as yellow oil. LCMS: m/z=252 [M+1]⁺

### Step 4. 7-chloro-2-iodo-3-(2,2,2-trifluoroethyl)thieno[3,2-b]pyridine

Into a 100-mL three necked purged and maintained with an inert atmosphere of nitrogen, was placed 7-chloro-3-(2,2,2-trifluoroethyl)thieno[3,2-b]pyridine (505.00 mg, 2.01 mmol), THF (10 mL). The reaction mixture was cooled to −70° C. LDA (2.0 M in THF) (2.50 mL, 5.00 mmol) was added and stirred at the same temperature for 0.5 h. 12 (1.41 g, 5.57 mmol) in THF (5 mL) was added to the reaction at −70° C. The reaction mixture was stirred at −70° C. for 0.5 h. LCMS showed that the reaction was completed. The resulting solution was quenched with H₂O (30 mL) and extracted with EA (2×50 mL). The organic layers combine d and washed with brine (30 mL), dried over anhydrous Na₂SO₄, the residue was concentrated under vacuum. The residue was applied onto a silica gel column eluted with EA:hexane (v:v=1:1). This resulted in 600.00 mg (79.19% yield) of 7-chloro-2-iodo-3-(2,2,2-trifluoroethyl)thieno[3,2-b]pyridine as brown solid. LCMS: m/z=378 [M+1]⁺

### Step 5. 2-iodo-N-(1-methylpiperidin-4-yl)-3-(2,2,2-trifluoroethyl)thieno[3,2-b]pyridin-7-amine

Into a 40-mL reaction vial purged and maintained with an inert atmosphere of nitrogen, was placed 7-chloro-2-iodo-3-(2,2,2-trifluoroethyl)thieno[3,2-b]pyridine (600.00 mg, 1.59 mmol), 1-methylpiperidin-4-amine (5.10 g, 44.65 mmol), DIEA (1.18 g, 9.12 mmol), NMP (5 mL). The reaction mixture was stirred at 120° C. for 12 h. LCMS showed that the reaction was completed. The resulting solution was cooled to room temperature and quenched with water (50 mL) and extracted with EA (3×50 mL). The organic layers combined and washed with brine (30 mL), dried over anhydrous Na₂SO₄, the residue was concentrated under vacuum. The residue was applied onto a C₁₈ column eluted with ACN:H₂O (0.01 TFA) (v:v=1:1), Flow rate: 80 mL/min. This resulted in 100.00 mg (13.82% yield) of 2-iodo-N-(1-methylpiperidin-4-yl)-3-(2,2,2-trifluoroethyl)thieno[3,2-b]pyridin-7-amine as yellow solid. LCMS: m/z=456 [M+1]⁺

### Step 6. (3-methoxy-4-((3-(7-((1-methylpiperidin-4-yl)amino)-3-(2,2,2-trifluoroethyl)thieno[3,2-b]pyridin-2-yl) prop-2-yn-1-yl)amino)phenyl)dimethylphosphine oxide (71)

Into a 20-mL reaction vial purged and maintained with an inert atmosphere of nitrogen, was placed 2-iodo-N-(1-methylpiperidin-4-yl)-3-(2,2,2-trifluoroethyl)thieno[3,2-b]pyridin-7-amine (100 mg, 0.22 mmol), Pd(PPh₃)₂Cl₂ (35.00 mg, 0.50 mmol), DIEA (328.00 mg, 2.54 mmol), CuI (38.00 mg, 0.20 mmol), DMSO (5 mL), (3-methoxy-4-(prop-2-yn-1-ylamino)phenyl)dimethylphosphine oxide (166.00 mg, 0.70 mmol). The reaction mixture was stirred at 50° C. for 3 h. LCMS showed that the reaction was completed. The resulting crude product was further purified by pre-HPLC with ACN/H₂O (0.1% ammonium hydroxide), Flow rate: 70 mL/min; Gradient: 35-70-70% B (2-37-46 min; 266 nm; RT: 36.01-42.90). This resulted in 57.00 mg (45.96% yield) of (3-methoxy-4-((3-(7-((1-methylpiperidin-4-yl)amino)-3-(2,2,2-trifluoroethyl)thieno[3,2-b]pyridin-2-yl)prop-2-yn-1-yl)amino)phenyl)dimethylphosphine oxide (71) as white solid. LCMS: m/z=565 [M+1]⁺

¹H NMR (400 MHz, DMSO-d₆) δ 8.29-8.24 (m, 1H), 7.29-7.17 (m, 1H), 7.14 (d, J=11.8 Hz, 1H), 6.87-6.80 (m, 1H), 6.67 (d, J=7.8 Hz, 1H), 6.62-6.58 (m, 1H), 6.08-6.01 (m, 1H), 4.35 (d, J=6.2 Hz, 2H), 3.85 (s, 3H), 3.75-3.70 (m, 2H), 3.56 (s, 1H), 2.99 (s, 2H), 2.34 (d, J=31.3 Hz, 5H), 1.94 (d, J=12.2 Hz, 2H), 1.69-1.55 (m, 8H).

### Example 72

### (Z)—(4-((3-(7-((3-fluoro-1′-methyl-[1,4′-bipiperidin]-4-yl)amino)-3-(2,2,2-trifluoroethyl)benzo[b]thiophen-2-yl) prop-2-yn-1-yl)amino)-3-methoxyphenyl)dimethylphosphine oxide(racemic) (72)

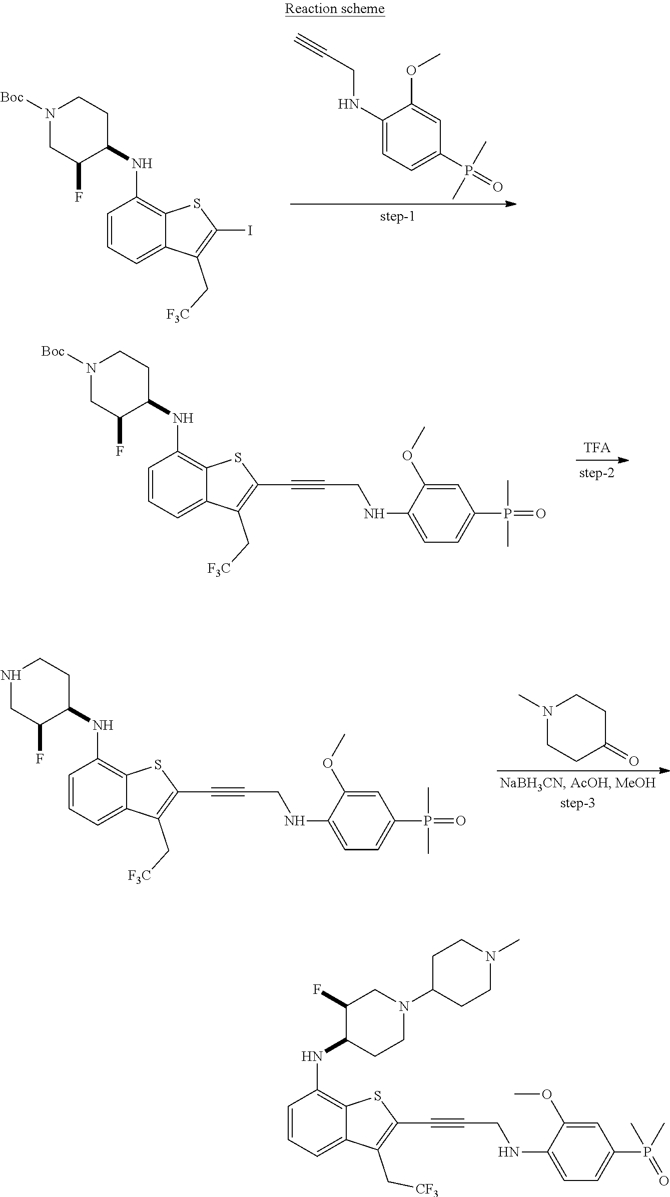

### Step 1. (Z)-tert-butyl 4-((2-(3-((4-(dimethylphosphoryl)-2-methoxyphenyl)amino)prop-1-yn-1-yl)-3-(2,2,2-tri fluoroethyl)benzo[b]thiophen-7-yl)amino)-3-fluoropiperidine-1-carboxylate

Into a 100-mL three necked bottle purged and maintained with an inert atmosphere of nitrogen, was placed (Z)-tert-butyl 3-fluoro-4-((2-iodo-3-(2,2,2-trifluoroethyl)benzo[b]thiophen-7-yl)amino)piperidine-1-carboxylate (1.52 g, 2.72 mmol), (3-methoxy-4-(prop-2-yn-1-ylamino)phenyl)dimethylphosphine oxide (1.53 g, 6.45 mmol), Pd(PPh₃)₂Cl₂ (224.00 mg, 0.32 mmol), CuI (310.00 mg, 1.63 mmol), DIEA (1.12 g, 8.67 mmol), DMF (20 mL). The reaction mixture was stirred at room temperature overnight. The residue was applied onto a silica gel column eluted with ACN:H₂O (0.01% TFA) (v:v=1:1), Flow rate: 100 mL/min. This resulted in 1. 50 g (82.74% yield) of (Z)-tert-butyl 4-((2-(3-((4-(dimethylphosphoryl)-2-methoxyphenyl)amino)prop-1-yn-1-yl)-3-(2,2,2-trifluoroethyl)benzo[b]thiophen-7-yl)amino)-3-fluoropiperidine-1-carboxylate as yellow solid. LCMS: m/z=668 [M+1]⁺

### Step 2. (4-((3-(7-(((Z)-3-fluoropiperidin-4-yl)amino)-3-(2,2,2-trifluoroethyl)benzo[b]thiophen-2-yl)prop-2-yn-1-yl)amino)-3-methoxyphenyl)dimethylphosphine oxide

Into a 100-mL three necked bottle purged and maintained with an inert atmosphere of nitrogen, was placed (Z)-tert-butyl 4-((2-(3-((4-(dimethylphosphoryl)-2-methoxyphenyl)amino)prop-1-yn-1-yl)-3-(2,2,2-trifluoroethyl) benzo[b]thiophen-7-yl)amino)-3-fluoropiperidine-1-carboxylate (1.50 g, 2.25 mmol), TFA (10 mL), DCM (30 mL), The reaction mixture was stirred at room temperature for 2 h. LCMS showed that the reaction was completed. The resulting solution was concentrated under vacuum. The residue was applied onto a silica gel column eluted with ACN:H₂O (0.01% TFA) (v:v=1:1), Flow rate: 100 m/min. This resulted in 1.20 g (93.79% yield) of (4-((3-((Z)-7-((3-fluoropiperidin-4-yl)amino)-3-(2,2,2-trifluoroethyl)benzo[b]thiophen-2-yl)prop-2-yn-1-yl)amino)-3-methoxyphenyl)dimethylphosphine oxide as yellow solid. LCMS: m/z=568 [M+1]⁺

### Step 3. (Z)-(4-((3-(7-((3-fluoro-1′-methyl-[1,4′-bipiperidin]-4-yl)amino)-3-(2,2,2-trifluoroethyl)benzo[b]thiophen-2-yl)prop-2-yn-1-yl)amino)-3-methoxyphenyl)dimethylphosphine oxide(racemic) (72)

Into a 8-mL reaction vial purged and maintained with an inert atmosphere of nitrogen, was placed (4-((3-(7-(((Z)-3-fluoropiperidin-4-yl)amino)-3-(2,2,2-trifluoroethyl)benzo[b]thiophen-2-yl)prop-2-yn-1-yl)amino)-3-methoxyphenyl)dimethylphosphine oxide (116.00 mg, 0.20 mmol), 1-methylpiperidin-4-one (118.00 mg, 1.04 mmol)), Acetic acid (161.00 mg, 2.68 mmol), MeOH (2 mL), Sodium cyanoboronhydride (120.00 mg, 2.80 mmol). The reaction mixture was stirred at room temperature for 12 h. LCMS showed that the reaction was completed. The resulting solution was concentrated under vacuum and the residue was quenched with sodium bicarbonate aqueous solution (20 mL), extracted with EA (2×30 mL). The organic layers combined and washed with brine (100 mL), dried over anhydrous Na₂SO₄, the residue was concentrated under vacuum. The resulting crude product was further purified by pre-HPLC with ACN/H₂O (0.1% ammonium hydroxide), Flow rate: 40 mL/min; Gradient: 20-50-70% B (2-30-60 min); 254 nm; RT: 32.808-37.321). This resulted in 36.00 mg (26.49% yield) of (Z)-(4-((3-(7-((3-fluoro-1′-methyl-[1,4′-bipiperidin]-4-yl)amino)-3-(2,2,2-trifluoroethyl)benzo[b]thiophen-2-yl)prop-2-yn-1-yl)amino)-3-methoxyphenyl)dimethylphosphine oxide(racemic) (72) as white solid. LCMS: m/z=665 [M+1]⁺

¹H NMR (400 MHz, DMSO-d₆) δ 7.34-7.18 (m, 3H), 7.15 (d, J=11.8 Hz, 1H), 6.88-6.83 (m, 1H), 6.78 (d, J=7.7 Hz, 1H), 6.07 (t, J=6.3 Hz, 1H), 5.15 (d, J=8.5 Hz, 1H), 4.80 (d, J=50.0 Hz, 1H), 4.34 (d, J=6.3 Hz, 2H), 3.94-3.74 (m, 5H), 3.74-3.57 (m, 1H), 3.13-3.10 (m, 1H), 2.88-2. 83 (m, 3H), 2.44 (d, J=12.6 Hz, 1H), 2.37-2.13 (m, 5H), 1.89-1.83 (m, 3H), 1.80-1.54 (m, 9H), 1.48-1.42 (m, 2H).

### Example 73

### (Z)-(4-((3-(7-((3-fluoro-1-(tetrahydro-2H-pyran-4-yl)piperidin-4-yl)amino)-3-(2,2,2-trifluoroethyl)benzo[b]thiophen-2-yl)prop-2-yn-1-yl)amino)-3-methoxyphenyl)dimethylphosphine oxide(racemic) (73)

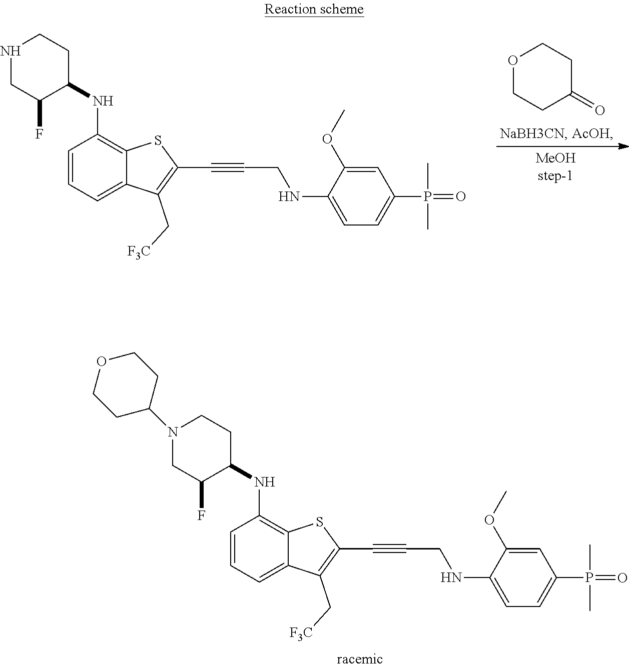 [SMILES: COc1cc(P(C)(C)=O)ccc1NCC#Cc1sc2c(N[C@@H]3CCNC[C@@H]3F)cccc2c1CC(F)(F)F ; O=C1CCOCC1 ; COc1cc(P(C)(C)=O)ccc1NCC#Cc1sc2c(N[C@@H]3CCN(C4CCOCC4)C[C@@H]3F)cccc2c1CC(F)(F)F]

### Experimental Details

### Step 1. (Z)-(4-((3-(7-((3-fluoro-1-(tetrahydro-2H-pyran-4-yl)piperidin-4-yl)amino)-3-(2,2,2-trifluoroethyl)benzo[b]thiophen-2-yl)prop-2-yn-1-yl)amino)-3-methoxyphenyl)dimethylphosphine oxide(racemic) (73)

Into a 8-mL reaction vial purged and maintained with an inert atmosphere of nitrogen, was placed (Z)-(4-((3-(7-((3-fluoropiperidin-4-yl)amino)-3-(2,2,2-trifluoroethyl)benzo[b]thiophen-2-yl)prop-2-yn-1-yl)amino)-3-methoxyphenyl)dimethylphosphine oxide (111.00 mg, 0.20 mmol), tetrahydro-4H-pyran-4-one (137.00 mg, 1.37 m mol), Sodium cyanoboronhydride (120.00 mg, 2.80 mmol), Acetic acid (164.00 mg, 2.73 mmol), MeOH (2 mL). The reaction mixture was stirred at room temperature for 12 h. LCMS showed that the reaction was completed. The resulting solution was concentrated under vacuum and the residue was quenched with sodium bicarbonate aqueous solution (20 mL), extracted with EA (2×30 mL). The organic layers combined and washed with brine (100 mL), dried over anhydrous Na₂SO₄, the residue was concentrated under vacuum. The resulting crude product was further purified by pre-HPLC with ACN/H₂O (0.1% ammonium hydroxide), Flow rate: 40 mL/min; Gradient: 20-55-55% B (2-30-60 min); 254 nm; RT: 29.283-31.138). This resulted in 38.00 mg (29.81% yield) of (Z)-(4-((3-(7-((3-fluoro-1-(tetrahydro-2H-pyran-4-yl)piperidin-4-yl)amino)-3-(2,2,2-trifluoroethyl)benzo[b]thiophen-2-yl)prop-2-yn-1-yl)amino)-3-methoxyphenyl)dimethylphosphine oxide (racemic) (73) as white solid. LCMS: m/z=652 [M+1]⁺

¹H NMR (400 MHz, DMSO-d₆) δ 7.28-7.20 (m, 3H), 7.17-7.09 (m, 1H), 6.88-6.82 (m, 1H), 6.79 (d, J=7.7 Hz, 1H), 6.07 (t, J=6.3 Hz, 1H), 5.12 (d, J=8.5 Hz, 1H), 4.81 (d, J=49.7 Hz, 1H), 4.3 (d, J=6.3 Hz, 2H), 3.97-3.60 (m, 8H), 3.29-3.23 (m, 2H), 3.16-3.11 (m, 1H), 2.90 (d, J=11.0 Hz, 1H), 2.53 (d, J=12.6 Hz, 1H), 2.48-2.42 (m, 1H), 2.35-2.25 (m, 1H), 1.95-1.83 (m, 1H), 1.80-1.70 (m, 1H), 1.63-1.53 (m, 8H), 1.50-1.35 (m, 2H).

### Example 74

### (4-((3-(7-(((Z)-1-cyclopropyl-3-fluoropiperidin-4-yl)amino)-3-(2,2,2-trifluoroethyl)benzo[b]thiophen-2-yl)prop-2-yn-1-yl)amino)-3-methoxyphenyl)dimethylphosphine oxide(racemic) (74)

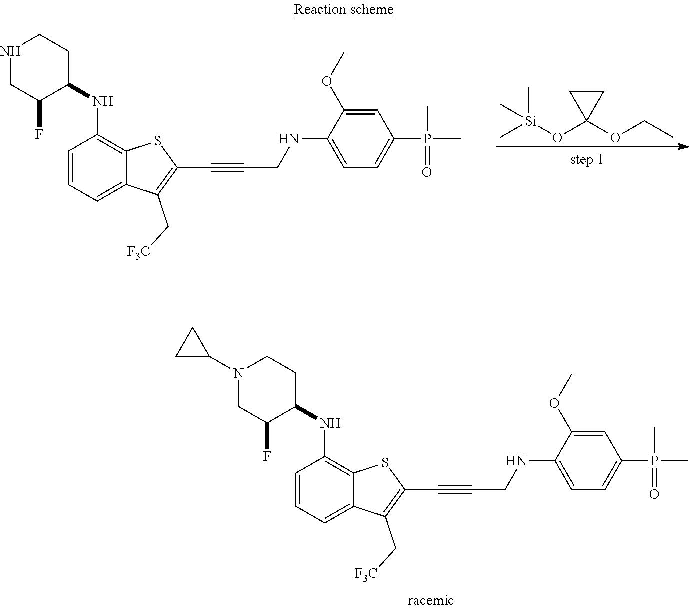

### Experimental Details

### Step 1. (4-((3-(7-(((Z)-1-cyclopropyl-3-fluoropiperidin-4-yl)amino)-3-(2,2,2-trifluoroethyl)benzo[b]thiophen-2-yl)prop-2-yn-1-yl)amino)-3-methoxyphenyl)dimethylphosphine oxide(racemic) (74)

Into a 8-mL sealed tube purged and maintained with an inert atmosphere of nitrogen, was placed 1-ethoxy-1-(trimethylsiloxy)cyclopropane (0.068 g, 390.1049 μmol), (4-((3-(7-(((Z)-3-fluoropiperidin-4-yl)amino)-3-(2,2,2-trifluoroethyl)benzo[b]thiophen-2-yl)prop-2-yn-1-yl)amino)-3-methoxyphenyl)dimethylphosphine oxide (0.051 g, 89.86 μmol), sodium cyanoboronhydride (0.033 g, 769.67 μmol), acetic acid (0.068 g, 1.13 mmol), methanol (2 mL). The reaction mixture was stirred at 50° C. for 17 h. The reaction was quenched by the addition of water (5 mL) and extracted with EA (2×5 mL). The organic layer was combined, washed with brine (5 mL), dried over anhydrous Na₂SO₄ and concentrated under vacuum. The mixture was purified by preparative HPLC (Mobile Phase A: water (10 mmoL/L ammonium hydroxide), Mobile Phase B: ACN; Flow rate: 40 mL/min; Gradient: 30-55-55% B (2-30-60 min); 266 nm; RT: 22.072-23.782 min). This resulted in 0.027 g (49.45% yield) of (4-((3-(7-(((Z)-1-cyclopropyl-3-fluoropiperidin-4-yl)amino)-3-(2,2,2-trifluoroethyl) benzo[b]thiophen-2-yl)prop-2-yn-1-yl)amino)-3-methoxyphenyl)dimethylphosphine oxide(racemic) (74) as yellow solid. LCMS: m/z=608 [M+1]⁺.

¹H NMR (400 MHz, DMSO) δ 7.31-7.23 (m, 1H), 7.22 (s, 1H), 7.20 (s, 1H), 7.14 (d, J=11.9 Hz, 1H), 6.87-6.82 (m, 1H), 6.79 (d, J=7.7 Hz, 1H), 6.05 (t, J=6.4 Hz, 1H), 5.09 (d, J=8.6 Hz, 1H), 4.86 (s, 1H), 4.73 (s, 1H), 4.34 (d, J=6.2 Hz, 2H), 3.85 (s, 3H), 3.81-3.71 (m, 2H), 3.66 (s, 1H), 3.16 (t, J=10.5 Hz, 1H), 2.96 (d, J=10.6 Hz, 1H), 2.37 (t, J=11.3 Hz, 1H), 1.92-1.78 (m, 1H), 1.70 (d, J=24.9 Hz, 2H), 1.58 (d, J=13.2 Hz, 6H), 0.43 (d, J=6.3 Hz, 2H), 0.32 (d, J=9.4 Hz, 1H), 0.25 (d, J=8.5 Hz, 1H).

### Example 75

### (4-((3-(7-(((Z)-3-fluoro-1-(oxetan-3-yl)piperidin-4-yl)amino)-3-(2,2,2-trifluoroethyl)benzo[b]thiophen-2-yl)prop-2-yn-1-yl)amino)-3-methoxyphenyl)dimethylphosphine oxide (75)

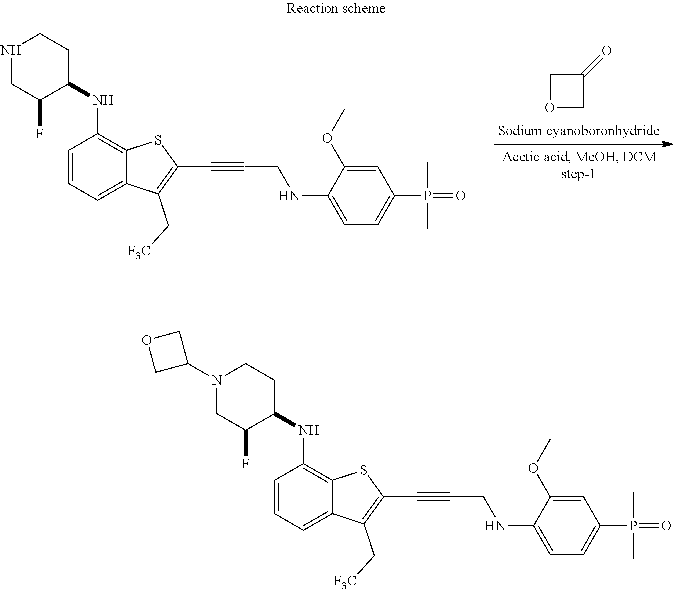 [SMILES: COc1cc(P(C)(C)=O)ccc1NCC#Cc1sc2c(N[C@@H]3CCNC[C@@H]3F)cccc2c1CC(F)(F)F ; O=C1COC1 ; COc1cc(P(C)(C)=O)ccc1NCC#Cc1sc2c(N[C@@H]3CCN(C4COC4)C[C@@H]3F)cccc2c1CC(F)(F)F]

### Experimental Details

### Step 1. (4-((3-(7-(((Z)-3-fluoro-1-(oxetan-3-yl)piperidin-4-yl)amino)-3-(2,2,2-trifluoroethyl)benzo[b]thiophen-2-yl)prop-2-yn-1-yl)amino)-3-methoxyphenyl)dimethylphosphine oxide (75)

Into a 8-mL sealed tube and maintained with an inert atmosphere of nitrogen, was placed (4-((3-(7-(((Z)-3-fluoropiperidin-4-yl)amino)-3-(2,2,2-trifluoroethyl)benzo[b]thiophen-2-yl)prop-2-yn-1-yl)amino)-3-methoxyphenyl) dimethylphosphine oxide (0.086 g, 151.52 μmol), oxetan-3-one (0.022 g, 305.29 μmol), Acetic acid (0.1 mL), methanol (0.6 mL), DCM (0.6 mL). Then Sodium cyanoboronhydride (0.119 g, 2.77 mmol) was added. The mixture was stirred at rt for 16 h. The reaction mixture was filtered through celite pad and celite pad was washed with ACN (3×20 mL). The filterate was then concentrated under reduced pressure to afford the crude product. The reaction mixture was purified by C18 column eluted with ACN/H₂O (v/v=1/3), filtered and concentrated under vacuum. This resulted in 0.031 g, (32.80% yield) of (4-((3-(7-(((Z)-3-fluoro-1-(oxetan-3-yl)piperidin-4-yl)amino)-3-(2,2,2-trifluoroethyl)benzo[b]thiophen-2-yl)prop-2-yn-1-yl)amino)-3-methoxyphenyl) dimethylphosphine oxide (75) as yellow solid. LCMS: m/z=624 [M+1]⁺.

¹H NMR (400 MHz, DMSO-d₆) δ 7.36-7.09 (m, 4H), 6.91-6.71 (m, 2H), 6.07 (t, J=6.4 Hz, 1H), 5.22 (s, 1H), 4.85 (d, J=49.3 Hz, 1H), 4.63-4.31 (m, 6H), 3.82 (d, J=27.9 Hz, 6H), 2.99 (s, 1H), 2.77 (s, 1H), 2.11 (d, J=101.7 Hz, 3H), 1.77 (d, J=10.7 Hz, 1H), 1.59 (d, J=13.1 Hz, 6H), 1.28 (m, 1H).

### Example 76

### 4-(4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)-1-methyl-1,4-azaphosphinane 4-oxide(racemic) (76)

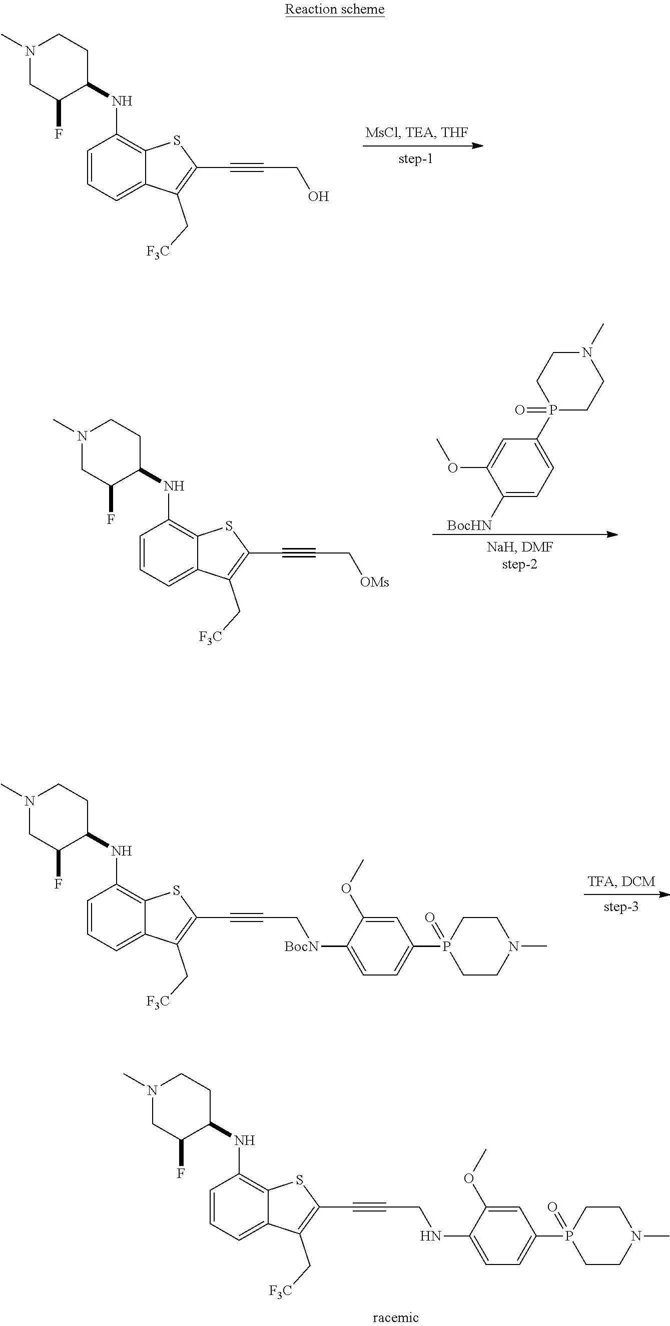

### Experimental Details

### Step 1. 3-(7-(((Z)-3-fluoro-1-methylpiperidin-4-yl)amino)-3-(2,2,2-trifluoroethyl)benzo[b]thiophen-2-yl)prop-2-yn-1-yl methanesulfonate

Into a 20-mL reaction vial purged and maintained with an inert atmosphere of nitrogen, was placed 3-(7-(((Z)-3-fluoro-1-methylpiperidin-4-yl)amino)-3-(2,2,2-trifluoroethyl)benzo[b]thiophen-2-yl)prop-2-yn-1-ol (311.00 mg, 0.77 mmol), THF (3 mL), TEA (212.00 g, 2.09 mmol). The reaction mixture was stirred at 0° C. Then MsCl (154.00 mg, 1.34 mmol) was added. The mixture was stirred at room temperature for 2 h. The reaction mixture was directly used in the next step. LCMS: m/z=479 [M+1]⁺

### Step 2. tert-butyl (3-(7-(((Z)-3-fluoro-1-methylpiperidin-4-yl)amino)-3-(2,2,2-trifluoroethyl)benzo[b]thiophen-2-yl)prop-2-yn-1-yl)(2-methoxy-4-(1-methyl-4-oxido-1,4-azaphosphinan-4-yl)phenyl)carbamate

Into a 20-mL reaction vial purged and maintained with an inert atmosphere of nitrogen, was placed (Z)-ter t-butyl (2-methoxy-4-(1-methyl-4-oxido-1,4-azaphosphinan-4-yl)phenyl)carbamate (260.00 mg, 0.73 mmol), D MF (5 mL), NaH (82.00 mg, 3.42 mmol). The mixture was stirred at room temperature for 0.5 h. 3-(7-(((Z)-3-fluoro-1-methylpiperidin-4-yl)amino)-3-(2,2,2-trifluoroethyl)benzo[b]thiophen-2-yl)prop-2-yn-1-yl methanesulfonate (372.00 mg, 0.78 mmol) in THF (3 mL) was added to the reaction at 0° C. The reaction mixture was stirred at room temperature for 12 h. LCMS showed that the reaction was completed. The resulting solution was quenched with water (20 mL). The resulting solution was extracted with EA (2×30 mL). The organic layers combined and washed with brine (20 mL), dried over anhydrous Na₂SO₄, the residue was concentrated under vacuum. The residue was applied onto a C18 column eluted with ACN/H₂O (0.01% TFA) (v:v=1:1), Flow rate: 80 m/min. This resulted in 54.00 mg (9.42% yield) of tert-butyl (3-(7-(((Z)-3-fluoro-1-methylpiperidin-4-yl)amino)-3-(2,2,2-trifluoroethyl)benzo[b]thiophen-2-yl)prop-2-yn-1-yl)(2-methoxy-4-(1-meth yl-4-oxido-1,4-azaphosphinan-4-yl)phenyl)carbamate as brow solid. LCMS: m/z=737 [M+1]⁺

### Step 3. 4-(4-((3-(7-(((Z)-3-fluoro-1-methylpiperidin-4-yl)amino)-3-(2,2,2-trifluoroethyl)benzo[b]thiophen-2-yl) prop-2-yn-1-yl)amino)-3-methoxyphenyl)-1-methyl-1,4-azaphosphinane 4-oxide(racemic) (76)

Into a 8-mL reaction vial purged and maintained with an inert atmosphere of nitrogen, was placed tert-butyl (3-(7-(((Z)-3-fluoro-1-methylpiperidin-4-yl)amino)-3-(2,2,2-trifluoroethyl)benzo[b]thiophen-2-yl)prop-2-yn-1-yl) (2-methoxy-4-(1-methyl-4-oxido-1,4-azaphosphinan-4-yl)phenyl)carbamate (53.00 mg, 0.072 mmol), DCM (3 mL), TFA (0.5 mL). The reaction mixture was stirred at room temperature for 4 h. LCMS showed that the reaction was completed. The resulting solution was concentrated under vacuum. The resulting crude product was further purified by pre-HPLC with ACN/H₂O (0.1% ammonium hydroxide), Flow rate: 70 mL/min; Gradient: 30-65-85% B (2-30-46 min); 268 nm; RT: 39.33-45.15). This resulted in 9.00 mg (19.65% yield) of 4-(4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)-1-methyl-1,4-azaphosphinane 4-oxide (racemic) (76) as yellow solid. LCMS: m/z=637 [M+1]⁺

¹H NMR (400 MHz, DMSO-d₆) δ 8.14 (s, 1H), 7.41-7.32 (m, 2H), 7.29-7.23 (m, 1H), 7.18 (d, J=12.5 Hz, 1H), 6.88 (d, J=5.6 Hz, 1H), 6.79-6.73 (m, 1H), 5.59-5.50 (m, 3H), 5.14-4.87 (m, 3H), 4.19-4.00 (m, 4H), 3.90-3.75 (m, 6H), 3.44-3.38 (m, 2H), 3.07 (s, 1H), 2.85-2.61 (m, 3H), 2.44-2.21 (m, 4H), 2.15-1.95 (m, 2H), 1.89-1.83 (m, 1H), 1.28-1.23 (m, 1H).

### Example 77

### 5-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-6-methoxy-2,3-dihydrobenzo[d]isothiazole 1,1-dioxide(racemic) (77)

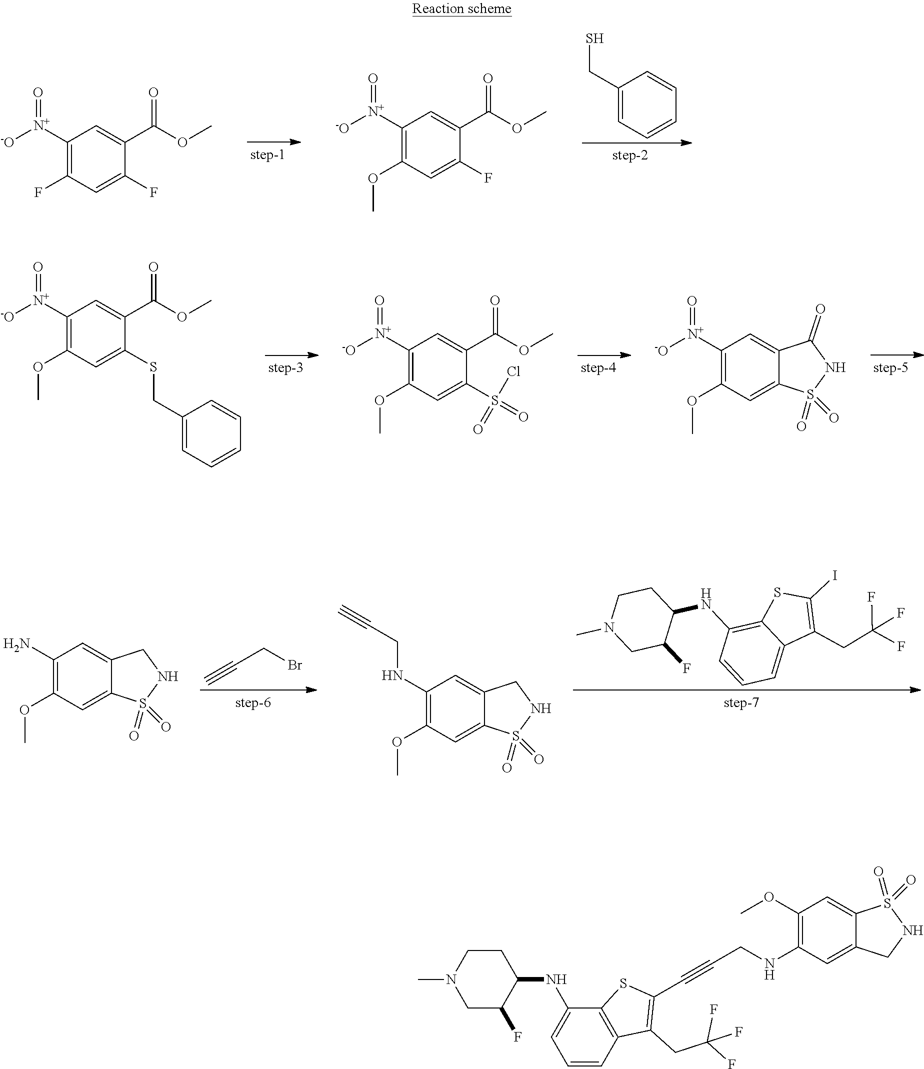

### Experiment Details

### Step 1. methyl 2-fluoro-4-methoxy-5-nitrobenzoate

Into a 500 mL round-bottom flask was placed methyl 2,4-difluoro-5-nitrobenzoate (5.01 g, 23.08 mmol) and MeOH (150 mL). The reaction was stirred at 0° C. MeON (1.38 g, 25.51 mmol) was added to the mixture slowly at 0° C. and stirred at 0° C. for 0.5 h. Warmed to R.T. and stirred for 2 h. The mixture was filtered and the filter cake was recrystallized with 50 mL trichloride carbon. The mixture was filtered and the filter cake was concentrated under vacuum. This resulted in 2.176 g (41.14% yield) of methyl 2-fluoro-4-methoxy-5-nitrobenzoate as off-white solid. LCMS: m/z=230[M+1]⁺.

### Step 2. methyl 2-(benzylthio)-4-methoxy-5-nitrobenzoate

Into a 50 mL round-bottom flask was placed methyl 2-fluoro-4-methoxy-5-nitrobenzoate (2.05 g, 8.95 mmol), K₂CO₃ (6.31 g, 45.67 mmol) and DMF (20 mL). Phenylmethanethiol (1.11 g, 8.94 mmol) was added to the mixture at 0° C. and stirred overnight. The reaction was quenched with water (100 mL), extracted with EA (2×100 mL). The organic layers were combined, washed with brine (100 mL), dried over anhydrous sodium sulfate, filtered and concentrated under vacuum. Add to MTBE (30 mL) and stirred at R.T. The mixture was filtered and the filter cake was dried under vacuum. This resulted in 2.83 g (94.87% yield) of methyl 2-(benzylthio)-4-methoxy-5-nitrobenzoate as yellow solid. LCMS: m/z=334[M+1]⁺.

### Step 3. methyl 2-(chlorosulfonyl)-4-methoxy-5-nitrobenzoate

Into a 100 mL round-bottom flask was placed methyl 2-(benzylthio)-4-methoxy-5-nitrobenzoate (2.79 g, 8.37 mmol), NCS (4.30 g, 32.23 mmol), Acetic acid (25 mL) and water (8 mL) at 0° c. The mixture was stirred at 0° m for 4 h. The reaction was added EA (100 ML), extracted with brine (3×100 ML). The organic layer was dried over anhydrous sodium sulfate, filtered and concentrated under vacuum. The crude was purified by silica gel column eluted with EA/hexane (V/V=1/1). This resulted in 3.054 g (crude) of methyl 2-(chlorosulfonyl)-4-methoxy-5-nitrobenzoate as yellow solid. LCMS: m/z=310[M+1]⁺.

### Step 4. 6-methoxy-5-nitrobenzo[d]isothiazol-3(2H)-one 1,1-dioxide

Into a 100 mL round-bottom flask was placed methyl 2-(chlorosulfonyl)-4-methoxy-5-nitrobenzoate (2.94 g, 9.48 mmol), Ammonium hydroxide (50 mL). The mixture was stirred at 30° C. for 4 h. The mixture was concentrated under vacuum. This resulted in 3.236 g (crude) of 6-methoxy-5-nitrobenzo[d]isothiazol-3(2H)-one 1,1-dioxide as green solid. LCMS: m/z=259[M+1]⁺.

### Step 5. 5-amino-6-methoxy-2,3-dihydrobenzo[d]isothiazole 1,1-dioxide

Into a 50 mL round-bottom flask was placed methyl 6-methoxy-5-nitrobenzo[d]isothiazol-3(2H)-one 1,1-diox ide (0.51 g, 1.47 mmol) and THF (10 mL). The reaction was stirred at 0° C. NaBH₄ (0.82 g, 21.78 mmol) and FeCl₃ (1.89 g, 11.66 mmol) were added to the mixture slowly at 0° C. and stirred at 70° C. for 3 h. The reaction was quenched with water (10 mL). Filtered and concentrated under vacuum. The residue purified by flash-C18 eluted with ACN/water (v/v=3/7) to provide the desired product. This resulted in 0.154 g (48.81% yield) of 5-amino-6-methoxy-2,3-dihydrobenzo[d]isothiazole 1,1-dioxide as off-white solid. LCMS: m/z=215[M+1]⁺.

### Step 6. 6-methoxy-5-(prop-2-yn-1-ylamino)-2,3-dihydrobenzo[d]isothiazole 1,1-dioxide

Into a 50 mL round-bottom flask was placed methyl 5-amino-6-methoxy-2,3-dihydrobenzo[d]isothiazole 1,1-dioxide (0.14 g, 0.68 mmol), 3-bromoprop-1-yne (0.10 g, 0.82 mmol), K₂CO₃ (0.29 g, 2.07 mmol), NaI (0.10 g, 0.69 mol) and DMF (10 mL). The reaction was stirred at 50° C. overnight. The residue purified by flash-C18 eluted with ACN/water (v/v=3/7) to provide the desired product. This resulted in 0.091 g (53.29% yield) of 6-methoxy-5-(prop-2-yn-1-ylamino)-2,3-dihydrobenzo[d]isothiazole 1,1-dioxide as yellow solid. LCMS: m/z=253[M+1]⁺.

### Step 7. 5-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-6-methoxy-2,3-dihydrobenzo[d]isothiazole 1,1-dioxide(racemic) (77)

Into a 8 mL vial was placed (Z)-3-fluoro-N-(2-iodo-3-(2,2,2-trifluoroethyl)benzo[b]thiophen-7-yl)-1-methylpiperidin-4-amine(racemic) (0.14 g, 0.29 mmol), 6-methoxy-5-(prop-2-yn-1-ylamino)-2,3-dihydrobenzo[d]isothiazole 1,1-dioxide (0.09 g, 0.36 mmol), bis(triphenylphosphine)palladium(II) chloride (0.04 g, 49.58 μmol), CuI (0.02 g, 78.76 μmol), DIEA (0.17 g, 1.30 mmol), Methyl sulfoxide (4 mL). The reaction was stirred at 60° C. for 3 h under nitrogen. The reaction was quenched with water (20 mL), extracted with EA (3×30 mL). The organic layers were combined, washed with brine (50 mL), dried over anhydrous sodium sulfate, filtered and concentrated under vacuum. The mixture was purified by preparative HPLC (Mobile Phase A: water (10 mmol/L ammonium hydroxide), Mobile Phase B: ACN; Flow rate: 70 mL/min; Gradient: 15-40-50% B (2-30-40 min); 266 nm; RT: 36.17-36.91 min) to provide the desired product. This resulted in 0.043 g (24.49% yield) of 5-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-6-methoxy-2,3-dihydrobenzo[d]isothiazole 1,1-dioxide(racemic) (77) as off-whit e solid. LCMS: m/z=597 [M+1]⁺.

¹H NMR (400 MHz, DMSO-d₆) δ 7.26-7.10 (m, 3H), 7.07 (s, 1H), 6.70 (d, J=7.8 Hz, 1H), 6.64 (s, 1H), 6.34 (t, J=6.2 Hz, 1H), 5.05 (d, J=8.6 Hz, 1H), 4.27 (d, J=5.8 Hz, 2H), 4.16 (s, 2H), 3.80 (s, 3H), 3.20 (s, 2H), 2.94 (t, J=11.6 Hz, 1H), 2.71 (d, J=10.9 Hz, 1H), 2.10 (s, 3H), 2.04-1.80 (m, 4H), 1.63 (d, J=12.5 Hz, 1H), 0.86-0.74 (m, 1H).

### Example 78

### N-(2-(3-((2-methoxy-4-(methylsulfonyl)phenyl)amino)prop-1-yn-1-yl)-3-(2,2,2-trifluoroethyl)benzo[b]thiophen-7-yl)-3,7-diazabicyclo[3.3.1]nonan-9-amine (78)

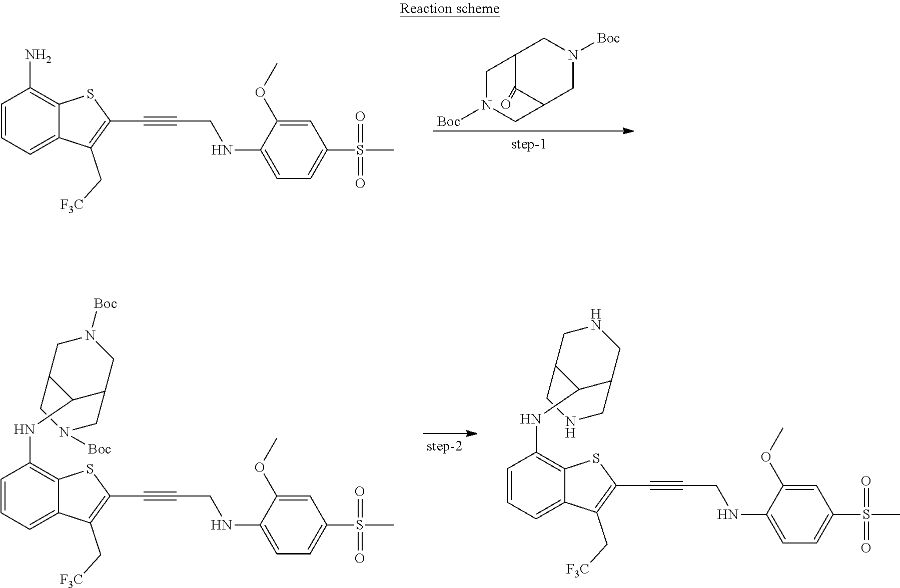 [SMILES: COc1cc(S(C)(=O)=O)ccc1NCC#Cc1sc2c(N)cccc2c1CC(F)(F)F ; CC(C)(C)OC(=O)N1CC2CN(C(=O)OC(C)(C)C)CC(C1)C2=O ; COc1cc(S(C)(=O)=O)ccc1NCC#Cc1sc2c(NC3C4CN(C(=O)OC(C)(C)C)CC3CN(C(=O)OC(C)(C)C)C4)cccc2c1CC(F)(F)F ; COc1cc(S(C)(=O)=O)ccc1NCC#Cc1sc2c(NC3C4CNCC3CNC4)cccc2c1CC(F)(F)F]

### Experiment Details

### Step 1. di-tert-butyl 9-((2-(3-((2-methoxy-4-(methylsulfonyl)phenyl)amino)prop-1-yn-1-yl)-3-(2,2,2-trifluoroethyl)benzo[b]thiophen-7-yl)amino)-3,7-diazabicyclo[3.3.1]nonane-3,7-dicarboxylate

Into a 8 mL vial was placed 2-(3-((2-methoxy-4-(methylsulfonyl)phenyl)amino)prop-1-yn-1-yl)-3-(2,2,2-trifluoroethyl)benzo[b]thiophen-7-amine (0.09 g, 0.20 mmol), di-tert-butyl 9-oxo-3,7-diazabicyclo[3.3.1]nonane-3,7-di carboxylate (0.13 g, 0.38 mmol), Titanium ethoxide (0.34 g, 0.15 mmol), Toluene (2 mL). The reaction w as stirred at 110° C. for 2 h under nitrogen. NaBH₃CN (0.07 g, 0.15 mmol) was added to the mixture and stirred at R.T for 2 h. The reaction was quenched with water (20 mL), extracted with EA (3×30 mL). The organic layers were combined, washed with brine (50 mL), dried over anhydrous sodium sulfate, filtered and concentrated under vacuum. The crude was purified by silica gel column eluted with EA/hexane (v/v=2/1). This resulted in 0.146 g (93.77% yield) of di-tert-butyl 9-((2-(3-((2-methoxy-4-(methylsulfonyl)phenyl)amino)prop-1-yn-1-yl)-3-(2,2,2-trifluoroethyl)benzo[b]thiophen-7-yl)amino)-3,7-diazabicyclo[3.3.1]nonane-3,7-dicarboxylate as brown oil. LCMS: m/z=793 [M+1]⁺.

### Step 2. N-(2-(3-((2-methoxy-4-(methylsulfonyl)phenyl)amino)prop-1-yn-1-yl)-3-(2,2,2-trifluoroethyl)benzo[b]thiophen-7-yl)-3,7-diazabicyclo[3.3.1]nonan-9-amine (78)

Into a 40 mL vial was placed di-tert-butyl9-((2-(3-((2-methoxy-4-(methylsulfonyl)phenyl)amino)prop-1-yn-1-yl)-3-(2,2,2-trifluoroethyl)benzo[b]thiophen-7-yl)amino)-3,7-diazabicyclo[3.3.1]nonane-3,7-dicarboxylate (0.14 g, 0.18 mmol) and hydrogen chloride (15 mL). The reaction was stirred at R.T for 4 h. PH was alkalized to 8 with KHCO3(aq.). The reaction was quenched with water (20 mL), extracted with EA (3×30 mL). The organic layers were combined, washed with brine (50 mL), dried over anhydrous sodium sulfate, filtered and concentrated under vacuum. The mixture was purified by preparative HPLC (Mobile Phase A: water (10 mmol/L ammonium hydroxide), Mobile Phase B:ACN; Flow rate: 70 mL/min; Gradient: 50-80-100% B (2-30-60 min); 269 nm; RT: 32.74-33.92) to provide the desired product. This resulted in 0.009 g (8.42% yield) of N-(2-(3-((2-methoxy-4-(methylsulfonyl)phenyl)amino)prop-1-yn-1-yl)-3-(2,2,2-trifluoroethyl)benzo[b]thiophen-7-yl)-3,7-diazabicyclo[3.3.1]nonan-9-amine (78) as brown solid. LCMS: m/z=593 [M+1]⁺.

¹H NMR (400 MHz, DMSO-d₆) δ 7.42-7.37 (m, 1H), 7.32-7.19 (m, 3H), 6.89 (d, J=8.4 Hz, 1H), 6.70 (t, J=7.5 Hz, 1H), 6.52 (t, J=6.3 Hz, 1H), 5.45-5.34 (m, 1H), 4.39 (d, J=6.3 Hz, 2H), 3.90 (s, 3H), 3.86-3.78 (m, 2H), 3.23 (m, 8H), 3.10 (s, 3H), 2.99 (t, J=11.4 Hz, 1H), 1.31-1.25 (m, 2H), 1.24 (d, J=3.6 Hz, 2H).

### Example 79

### N-cyclopropyl-4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-methoxybenzamide (79)

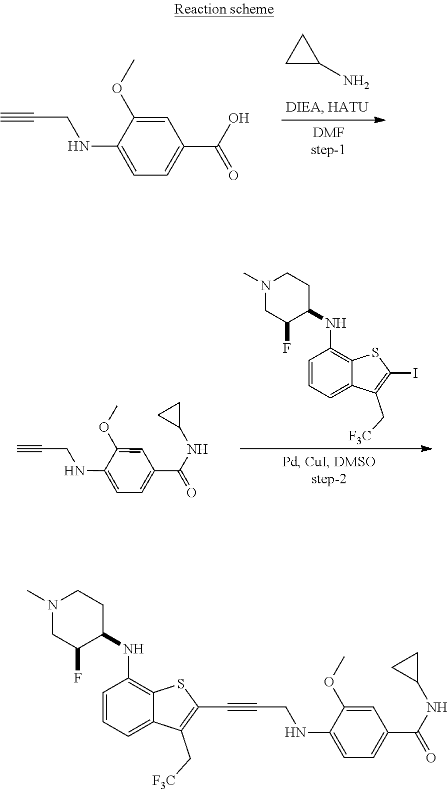

### Experimental Details

### Step 1. N-cyclopropyl-3-methoxy-4-(prop-2-yn-1-ylamino)benzamide

Into a 25-mL round-bottom flask purged and maintained with an inert atmosphere of nitrogen, was placed 3-methoxy-4-(prop-2-yn-1-ylamino)benzoic acid (0.416 g, 2.02 mmol), cyclopropanamine (0.222 g, 3.88 mmol), DIEA (0.222 g, 3.8883 mmol), TATU (1.300 g, 3.41 mmol), DMF (5 mL). The reaction mixture was stirred at room temperature for 1 h. The reaction mixture was filtered through celite pad and celite pad was washed with ACN (3×20 mL). The filterate was then concentrated under reduced pressure to afford the crude product. The reaction mixture was purified by C18 column eluted with ACN/H₂O (v/v=1/3), filtered and concentrated under vacuum. This resulted in 0.474 g, (95.71% yield) of N-cyclopropyl-3-methoxy-4-(prop-2-yn-1-ylamino)benzamide as brown oil. LCMS: m/z=245 [M+1]⁺.

### Step 2. N-cyclopropyl-4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-methoxybenzamide (79)

Into a 10-mL round-bottom flask was placed N-cyclopropyl-3-methoxy-4-(prop-2-yn-1-ylamino)benzamide (0.382 g, 1.56 mmol), (Z)-3-fluoro-N-(2-iodo-3-(2,2,2-trifluoroethyl)benzo[b]thiophen-7-yl)-1-methylpiperidin-4-amine (0.090 g, 190.56 μmol), Pd(PPh₃)₂Cl₂ (0.026 g, 36.83 μmol), CuI (0.019 g, 99.76 μmol), DIEA (0.100 g, 773.73 μmol), methyl sulfoxide (3 mL). The reaction was stirred under nitrogen atmosphere at RT for 16 h. The reaction mixture was filtered through celite pad and celite pad was washed with ACN (3×20 mL). The filterate was then concentrated under reduced pressure to afford the crude product. The crude product was purified by preparative HPLC (Mobile Phase A: water (0.1% ammonium hydroxide), Mobile Phase B: MeOH; Flow rate: 70 mL/min; Gradient: 50-85-95% B (2-30-45 min); 266 nm; RT: 33.346-34.917) to provide the desired product. This resulted in 0.076 g (67.74% yield) of N-cyclopropyl-4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-methoxybenzamide (79) as white solid. LCMS: m/z=589 [M+1]⁺.

¹H NMR (400 MHz, DMSO-d₆) δ 8.10 (d, J=4.0 Hz, 1H), 7.43 (d, J=8.2 Hz, 1H), 7.35-7.19 (m, 3H), 6.78-6.73 (m, 2H), 6.02 (t, J=6.3 Hz, 1H), 5.13 (d, J=8.5 Hz, 1H), 4.80 (d, J=49.4 Hz, 1H), 4.34 (d, J=6.3 Hz, 2H), 3.88-3.60 (m, 6H), 3.03 (t, J=11.3 Hz, 1H), 2.86-2.81 (m, 2H), 2.32-1.87 (m, 6H), 1.72 (d, J=12.1 Hz, 1H), 0.68-0.65 (m, 2H), 0.54 (t, J=3.3 Hz, 2H). The following compounds in the table can be synthesized by procedures similar to those described in Examples herein, using suitable starting materials and reagents.

| Example.No | Chemical structure |
|---|---|
| 80 | 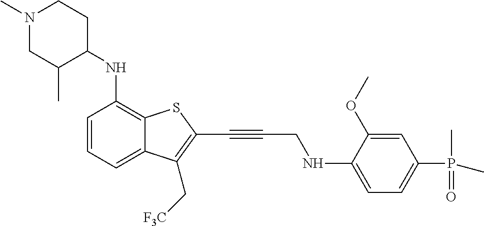 |
| 159 | 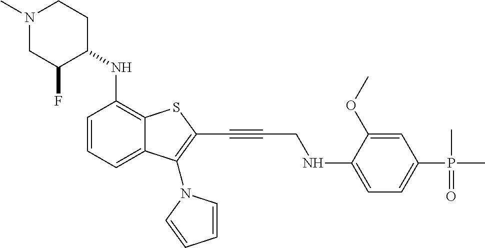 |
| 166 | 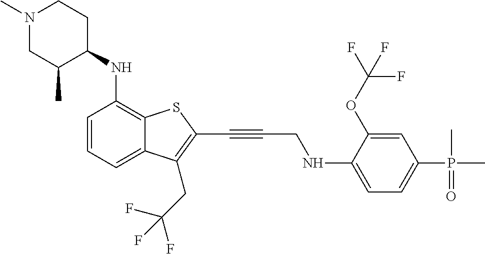 |
| 167 | 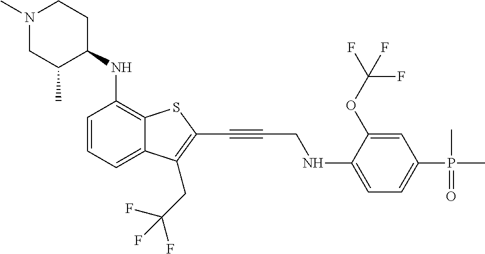 |
| 168 | 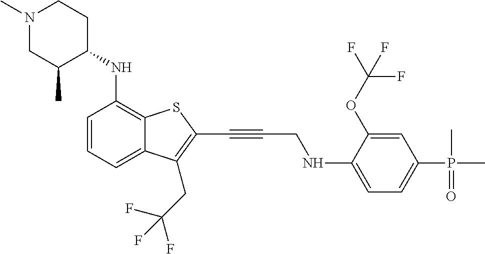 |
| 176 | 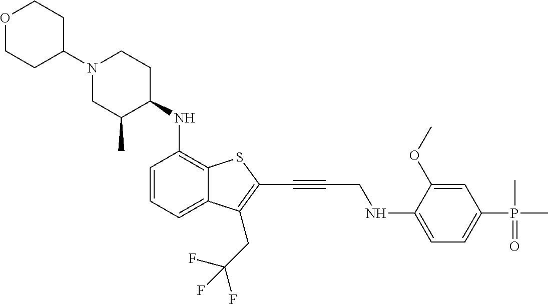 [SMILES: COc1cc(P(C)(C)=O)ccc1NCC#Cc1sc2c(N[C@@H]3CCN(C4CCOCC4)C[C@@H]3C)cccc2c1CC(F)(F)F] |
| 177 | 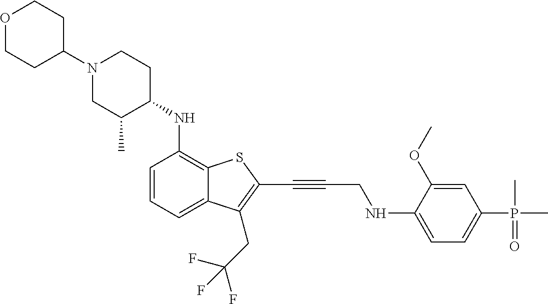 [SMILES: COc1cc(P(C)(C)=O)ccc1NCC#Cc1sc2c(N[C@H]3CCN(C4CCOCC4)C[C@H]3C)cccc2c1CC(F)(F)F] |
| 178 | 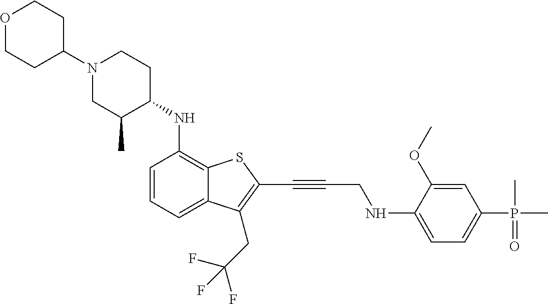 [SMILES: COc1cc(P(C)(C)=O)ccc1NCC#Cc1sc2c(N[C@H]3CCN(C4CCOCC4)C[C@@H]3C)cccc2c1CC(F)(F)F] |
| 190 | 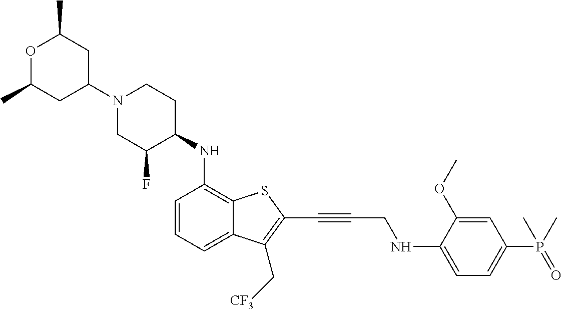 [SMILES: COc1cc(P(C)(C)=O)ccc1NCC#Cc1sc2c(N[C@@H]3CCN(C4C[C@@H](C)O[C@@H](C)C4)C[C@@H]3F)cccc2c1CC(F)(F)F] |
| 191 | 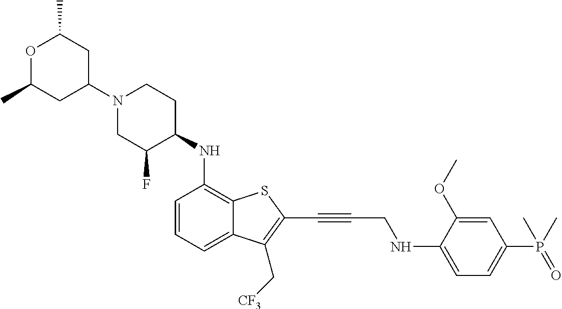 [SMILES: COc1cc(P(C)(C)=O)ccc1NCC#Cc1sc2c(N[C@@H]3CCN(C4C[C@@H](C)O[C@H](C)C4)C[C@@H]3F)cccc2c1CC(F)(F)F] |

### Example 81

### 4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-(2-methoxyethoxy)-N-methylbenzamide (racemic) (81)

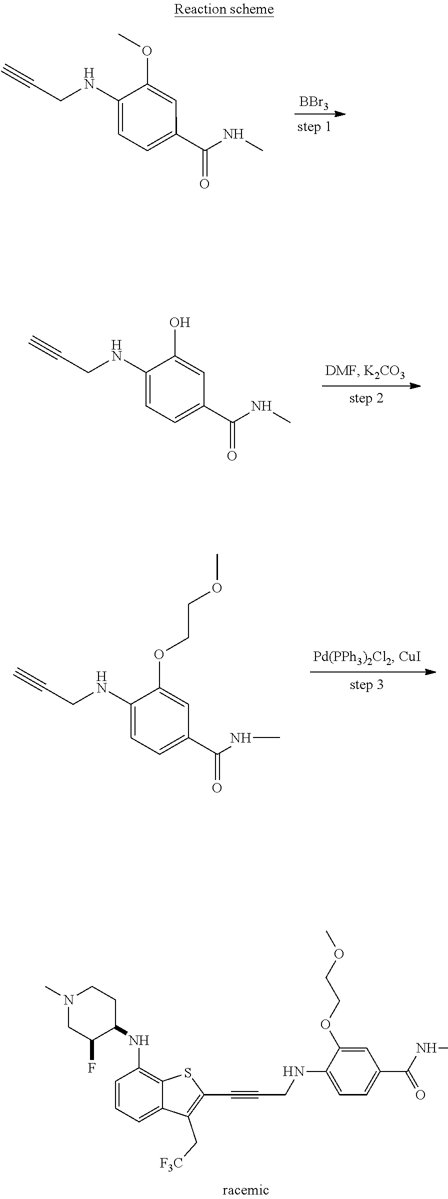

### Experimental Details

### Step 1. 3-hydroxy-N-methyl-4-(prop-2-yn-1-ylamino)benzamide

Into a 50-mL sealed tube purged and maintained with an inert atmosphere of nitrogen, was placed 3-methoxy-N-methyl-4-(prop-2-yn-1-ylamino)benzamide (1.005 g, 4.60 mmol), dichloromethane (5 mL), then tribromoboron (10 mL) was added at −20 mL)) ube purged and maintained with an inert atmosphere of nitrogen, w as placed 3-methoxy-N-methyl-odium hydroxide aqueous solution (2 M) to the mixture until pH=10˜1. The resulting solution was extracted with dichloromethane (2×10 mL). The organic layer was combined and hydrochloric acid aqueous solution (2 M) was added to the mixture until pH=7-8. The resulting solution was extracted with EA (2×20 mL). The organic layer was combined, washed with brine (20 mL), dried over anhydrous Na₂SO₄ and concentrated under vacuum. The residue was applied onto a silica gel column eluted with EA/hexane (v/v=4/5). This resulted in 0.49 g (52.10% yield) of 3-hydroxy-N-methyl-4-(prop-2-yn-1-ylamino)benzamide as yellow solid. LCMS: m/z=205 [M+1]⁺.

### Step 2. 3-(2-methoxyethoxy)-N-methyl-4-(prop-2-yn-1-ylamino)benzamide

Into a 8-mL sealed tube purged and maintained with an inert atmosphere of nitrogen, was placed 3-hydroxy-N-methyl-4-(prop-2-yn-1-ylamino)benzamide (0.101 g, 494.55 μmol), potassium carbonate (0.206 g, 1.49 mmol), 1-bromo-2-methoxyethane (0.111 g, 798.61 μmol), N,N-dimethylformamide (2 mL). The reaction mixture was stirred at 50 a for 16 h. The residues were purified by C₁₈ chromatography column eluted with ACN/H₂O (0.05% TFA) (v/v=2/5). This resulted in 0.059 g (45.48% yield) of 3-(2-methoxyethoxy)-N-methyl-4-(prop-2-yn-1-ylamino)benzamide as yellow solid. LCMS: m/z=263 [M+1]⁺.

### Step 3. 4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-(2-methoxyethoxy)-N-methylbenzamide (racemic) (81)

Into a 8-mL flask purged and maintained with an inert atmosphere of nitrogen, was placed (Z)-3-fluoro-N-(2-iodo-3-(2,2,2-trifluoroethyl)benzo[b]thiophen-7-yl)-1-methylpiperidin-4-amine (0.090 g, 190.56 μmol), methy sulfoxide (2 mL), 3-(2-methoxyethoxy)-N-methyl-4-(prop-2-yn-1-ylamino)benzamide (0.058 g, 221.12 μmol), [1,1′-bis(diphenylphosphino)ferrocene]dichloropalladium(II) (0.014 g, 19.83 μmol), cuprous iodide (0.006 g, 31.5043 μmol), triethylamine (0.030 g, 296.47 μmol). The reaction mixture was stirred at 50 refor 14 h. The reaction was quenched by the addition of water (10 mL) and extracted with EA (2×10 mL). The organic layer was combined, washed with brine (10 mL), dried over anhydrous Na₂SO₄ and concentrated under vacuum. The mixture was purified by preparative HPLC (Mobile Phase A: water (10 mmoL/L ammonium hydroxide), Mobile Phase B: MeOH; Flow rate: 70 mL/min; Gradient: 55-80-100% B (2-30-60 min); 266 n m; RT: 33.523-35.227 min). This resulted in 0.043 g (37.19% yield) of 4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-(2-methoxyethoxy)-N-methylbenzamide(racemic) (81) as off-white solid. LCMS: m/z=607 [M+1]⁺.

¹H NMR (400 MHz, DMSO) δ 8.10 (s, 1H), 7.44 (s, 1H), 7.36 (s, 1H), 7.32-7.17 (m, 2H), 6.78 (s, 2H), 5.86 (s, 1H), 5.15 (s, 1H), 4.87 (s, 1H), 4.74 (s, 1H), 4.38 (s, 2H), 4.15 (s, 2H), 3.82 (s, 2H), 3.73 (s, 2H), 3.02 (s, 2H), 2.76 (s, 4H), 2.19 (d, J=9.0 Hz, 3H), 2.07 (s, 1H), 1.95 (s, 1H), 1.72 (s, 1H), 1.25 (s, 1H).

### Example 82

### 4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-(2-(2-methoxyethoxy)ethoxy)-N-methylbenzamide(racemic) (82)

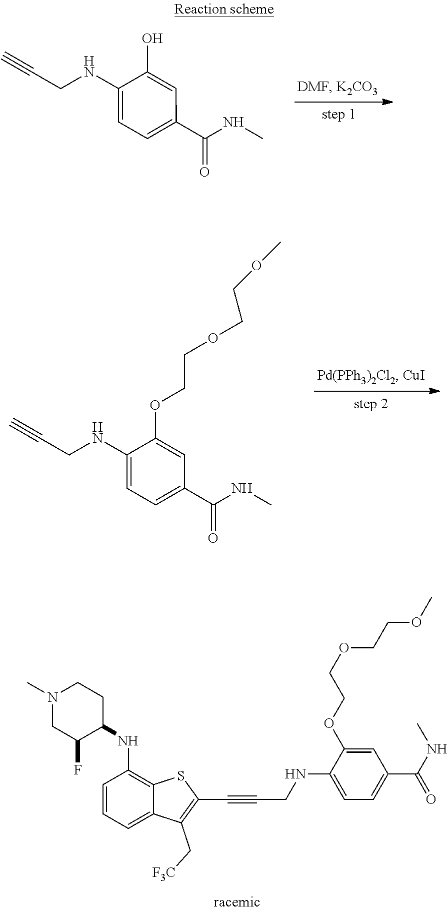

### Experimental Details

### Step 1. 3-(2-(2-methoxyethoxy)ethoxy)-N-methyl-4-(prop-2-yn-1-ylamino)benzamide

Into a 8-mL sealed tube purged and maintained with an inert atmosphere of nitrogen, was placed 3-hydroxy-N-methyl-4-(prop-2-yn-1-ylamino)benzamide (0.101 g, 494.55 μmol), 1-bromo-2-(2-methoxyethoxy)ethane (0.157 g, 857.72 μmol), dipotassium carbonate (0.266 g, 1.92 mmol), N,N-dimethylformamide (2 mL). The reaction mixture was stirred at 50 ained with anhe reaction mixture was purified by C₁₈ chromatography column eluted with ACN/H₂O (0.05% TFA) (v/v=2/5). This resulted in 0.112 g (73.92% yield) of 3-(2-(2-methoxyethoxy)ethoxy)-N-methyl-4-(prop-2-yn-1-ylamino)benzamide as yellow solid. LCMS: m/z=307 [M+1]⁺.

### Step 2. 4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-(2-(2-methoxyethoxy)ethoxy)-N-methylbenzamide(racemic) (82)

Into a 8-mL flask purged and maintained with an inert atmosphere of nitrogen, was placed (Z)-3-fluoro-N-(2-iodo-3-(2,2,2-trifluoroethyl)benzo[b]thiophen-7-yl)-1-methylpiperidin-4-amine (0.106 g, 224.44 μmol), methy sulfoxide (2 mL), 3-(2-(2-methoxyethoxy)ethoxy)-N-methyl-4-(prop-2-yn-1-ylamino)benzamide (0.118 g, 385.17 μmol), [1,1′-bis(diphenylphosphino)ferrocene]dichloropalladium(II) (0.015 g, 21.25 μmol), cuprous iodide (0.006 g, 31.50 μmol), triethylamine (0.057 g, 563.30 μmol). The reaction mixture was stirred at 50 refor 14 h. The reaction was quenched by the addition of water (10 mL) and extracted with EA (2×10 mL). The organic layer was combined, washed with brine (10 mL), dried over anhydrous Na₂SO₄ and concentrated under vacuum. The mixture was purified by preparative HPLC (Mobile Phase A: water (10 mmoL/L ammonium hydroxide), Mobile Phase B: MeOH; Flow rate: 70 mL/min; Gradient: 55-80-100% B (2-30-60 min); 266 nm; RT: 34.008-35.510 min). This resulted in 0.026 g (17.80% yield) of 4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-(2-(2-methoxy ethoxy)ethoxy)-N-methylbenzamide(racemic) (82) as off-white solid. LCMS: m/z=651 [M+1]⁺.

¹H NMR (400 MHz, DMSO) δ 8.09 (d, J=3.8 Hz, 1H), 7.44 (d, J=8.3 Hz, 1H), 7.37 (s, 1H), 7.27 (t, J=7.7 Hz, 1H), 7.20 (d, J=7.7 Hz, 1H), 6.78 (d, J=8.2 Hz, 2H), 5.86 (s, 1H), 5.14 (d, J=7.9 Hz, 1H), 4.37 (d, J=5.9 Hz, 2H), 4.14 (s, 2H), 3.80 (s, 2H), 3.64-3.59 (m, 2H), 3.50-3.45 (m, 2H), 3.29 (s, 10H), 3.25 (s, 3H), 2.75 (d, J=4.2 Hz, 3H), 2.18 (s, 3H).

### Example 83

### (3-chloro-4-((3-(7-(((Z)-3-fluoro-1-methylpiperidin-4-yl)amino)-3-(2,2,2-trifluoroethyl)benzo[b]thiophen-2-yl) prop-2-yn-1-yl)amino)phenyl)dimethylphosphine oxide (racemic) (83)

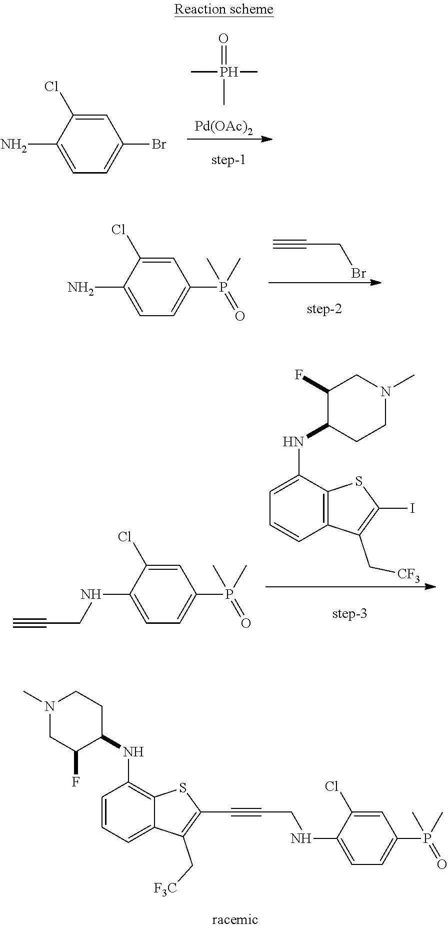

### Experimental Details

### Step 1. (4-amino-3-chlorophenyl)dimethylphosphine oxide

Into a 40-mL reaction vial purged and maintained with an inert atmosphere of nitrogen, was placed 4-bromo-2-chloroaniline (4.18 g, 20.25 mmol), Palladium (II) acetate (0.25 g, 1.150 mmol), Dimethylbisdiphenylphosphinoxanthene (1.34 g, 2.32 mmol), DIEA (4.29 g, 33.21 mmol), DMF (20 mL). The mixture was stir red at 110° C. for 5 min then dimethylphosphine oxide (1.174 g, 15.04 mmol) in THF (5 mL) was added to the reaction at 110° C. The reaction mixture was stirred at 110° C. for 3 h. The resulting solution was added to water (100 mL). The resulting solution was extracted with EA (2×50 mL). The aqueous layer was concentrated under vacuum. The residue was applied onto a C18 column eluted with ACN/H₂O (0.01% TFA) (v:v=1:1), Flow rate: 100 m/min. This resulted in 3.50 g (84.99% yield) of (4-amino-3-chlorophenyl) dimethylphosphine oxide as yellow oil. LCMS: m/z=204 [M+1]⁺

### Step 2. (3-chloro-4-(prop-2-yn-1-ylamino)phenyl)dimethylphosphine oxide

Into a 100-mL three necked bottle purged and maintained with an inert atmosphere of nitrogen, was placed (4-amino-3-chlorophenyl)dimethylphosphine oxide (3.12 g, 15.30 mmol), K₂CO₃ (7.38 g, 53.42 mmol), KI (4.63 g, 27.91 mmol), DMF (10 Ml), 3-bromoprop-1-yne (6.12 g, 51.45 mmol). The reaction mixture was stirred at 80° C. for 3 h. The residue was applied onto a C18 column eluted with ACN/H₂O (0.01% TFA) (v:v=1:1), Flow rate: 100 mL/min. This resulted in 121.00 mg (3.27% yield) of (3-chloro-4-(prop-2-yn-1-ylamino)phenyl)dimethylphosphine oxide as brown oil. LCMS: m/z=242 [M+1]⁺

### Step 3. (3-chloro-4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)phenyl)dimethylphosphine oxide (racemic) (83)

Into a 50-mL three necked bottle purged and maintained with an inert atmosphere of nitrogen, was placed (Z)-3-fluoro-N-(2-iodo-3-(2,2,2-trifluoroethyl)benzo[b]thiophen-7-yl)-1-methylpiperidin-4-amine (81.00 mg, 0.17 mmol), (3-chloro-4-(prop-2-yn-1-ylamino)phenyl)dimethylphosphine oxide (133.00 mg, 0.55 mmol), Pd(PPh₃)₂Cl₂ (22.00 mg, 0.032 mmol), CuI (29.00 mg, 0.15 mmol), DMSO (5 ml), DIEA (133.00 mg, 1.03 mmol). The reaction mixture was stirred at room temperature for 3 h. LCMS showed that the reaction was completed. The resulting crude product was further purified by pre-HPLC with MeOH/H₂O (0.1% ammonium hydroxide), Flow rate: 70 mL/min; Gradient: 55-80-100% B (2-30-60 min); 266 nm; RT:32.660-34.730). This resulted in 26.00 mg (25.86% yield) of (3-chloro-4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)phenyl)dimethylphosphine oxide (83) as white solid. LCMS: m/z=586 [M+1]⁺

¹H NMR (400 MHz, DMSO-d₆) δ 7.56 (d, J=11.2 Hz, 1H), 7.53-7.42 (m, 1H), 7.23-7.18 (m, 1H), 7.13 (d, J=7.9 Hz, 1H), 6.95 (d, J=8.2 Hz, 1H), 6.70 (d, J=7.6 Hz, 1H), 6.46 (t, J=6.0 Hz, 1H), 5.07 (d, J=8.4 Hz, 1H), 4.71 (d, J=49.6 Hz, 1H), 4.34 (d, J=5.5 Hz, 2H), 3.75-3.70 (m, 2H), 3.56 (d, J=29.4 Hz, 1H), 2.97-2.92 (m, 1H), 2.70 (d, J=10.6 Hz, 1H), 2.23-2.05 (m, 4H), 1.99-1.90 (m, 1H), 1.88-1.81 (m, 1H), 1.67-1.60 (m, 1H), 1.55-1.45 (m, 6H).

### Example 84

### (3-fluoro-4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)phenyl)dimethylphosphine oxide (84)

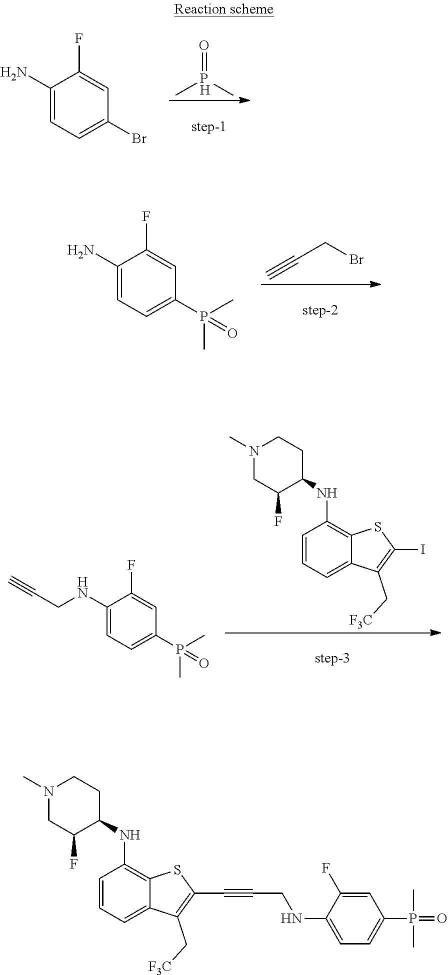

### Experiment Details

### Step 1. (4-amino-3-fluorophenyl)dimethylphosphine oxide

Into a 40 mL vial was placed 4-bromo-2-fluoroaniline (2.10 g, 11.03 mmol), Palladium (II) acetate (0.47 g, 2.09 mmol), Dimethylbisdiphenylphosphinoxanthene (1.22 g, 2.10 mmol), N,N-Diisopropylethylamine (4.83 g, 37.36 mmol), DMF (20 mL). The reaction was stirred at 130° C. for 0.5 h under nitrogen. Dimethylphosphine oxide (2.52 g, 32.33 mmol) was added to the mixture slowly. The reaction was stirred at 130° C. for 1 h under nitrogen. The residue was purified by flash-C18 eluted with ACN/water (V/V=1/4) to provide the desired product. This resulted in 3.528 g (crude) of (4-amino-3-fluorophenyl)dimethylphosphine oxide as yellow oil. LCMS: m/z=188 [M+1]⁺.

### Step 2. (3-fluoro-4-(prop-2-yn-1-ylamino)phenyl)dimethylphosphine oxide

Into a 100 mL round-bottom flask was placed methyl (4-amino-3-fluorophenyl)dimethylphosphine oxide (3.54 g, 18.90 mmol), 3-bromoprop-1-yne (1.82 g, 15.33 mmol), K₂CO₃ (5.29 g, 38.25 mmol), NaI (1.53 g, 10.21 mmol) and DMF (30 mL). The reaction was stirred at 50° C. overnight. The residue was purified by f lash-C18 eluted with ACN/water (V/V=3/7) to provide the desired product. This resulted in 0.892 g (20.95% yield) of (3-fluoro-4-(prop-2-yn-1-ylamino)phenyl)dimethylphosphine oxide as yellow oil. LCMS: m/z=226[M+1]⁺.

### Step 3. (3-fluoro-4-((3-(7-(((3S,4R)-3-fluoro-1-methylpiperidin-4-yl)amino)-3-(2,2,2-trifluoroethyl)benzo[b]thio phen-2-yl)prop-2-yn-1-yl)amino)phenyl)dimethylphosphine oxide (84)

Into a 20 mL vial was placed (3S,4R)-3-fluoro-N-(2-iodo-3-(2,2,2-trifluoroethyl)benzo[b]thiophen-7-yl)-1-meth ylpiperidin-4-amine (0.21 g, 0.45 mmol), (3-fluoro-4-(prop-2-yn-1-ylamino)phenyl)dimethylphosphine oxide (0.14 g, 0.62 mmol), bis(triphenylphosphine)palladium(II) chloride (0.06 g, 0.09 mmol), CuI (0.03 g, 0.13 mm ol), DIEA (0.21 g, 1.61 mmol), Methyl sulfoxide (5 mL). The reaction was stirred at 60° C. for 3 h under nitrogen. The reaction was quenched with water (20 mL), extracted with EA (3×30 mL). The organic layers were combined, washed with brine (50 mL), dried over anhydrous sodium sulfate, filtered and concentrated under vacuum. The mixture was purified by preparative HPLC (Mobile Phase A: water (10 mmol/L ammonium hydroxide), Mobile Phase B: ACN; Flow rate: 70 mL/min; Gradient: 15-40-60% B (2-30-40 min); 266 nm; RT: 36.23-36.95 min) to provide the desired product. This resulted in 0.046 g (17.91% yield) of (3-fluoro-4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)phenyl)dimethylphosphine oxide (84) as off-white solid. LCMS: m/z=570 [M+1]⁺.

¹H NMR (400 MHz, DMSO-d₆) δ 7.42 (t, J=9.9 Hz, 2H), 7.32-7.16 (m, 2H), 7.08-6.99 (m, 1H), 6.78 (d, J=7.7 Hz, 1H), 6.62 (t, J=6.3 Hz, 1H), 5.16 (d, J=8.5 Hz, 1H), 4.80 (d, J=49.5 Hz, 1H), 4.37 (d, J=6.2 Hz, 2H), 3.87-3.74 (m, 2H), 3.65 (d, J=29.2 Hz, 1H), 3.10-2.95 (m, 1H), 2.80 (d, J=11.0 Hz, 1H), 2.27 (d, J=13.1 Hz, 1H), 2.18 (s, 3H), 2.08 (t, J=11.7 Hz, 1H), 1.94 (d, J=12.8 Hz, 1H), 1.78-1.66 (m, 1H), 1.58 (d, J=13.2 Hz, 6H).

### Example 85

### 4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-methoxy-N-(2-methoxyethyl)benzamide (85)

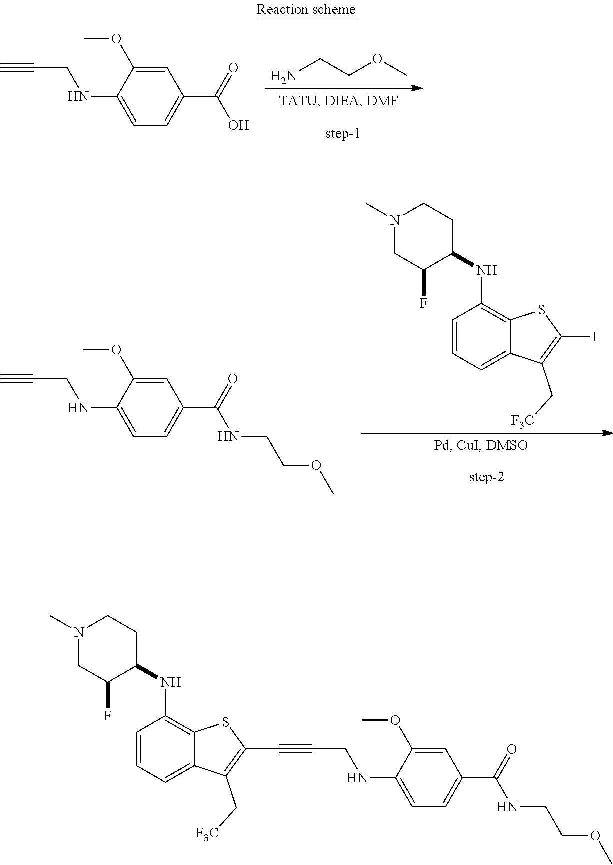

### Experimental Details

### Step 1. N-cyclopropyl-3-methoxy-4-(prop-2-yn-1-ylamino)benzamide

Into a 25-mL round-bottom flask purged and maintained with an inert atmosphere of nitrogen, was placed 3-methoxy-4-(prop-2-yn-1-ylamino)benzoic acid (0.328 g, 1.59 mmol), 2-methoxyethan-1-amine (0.180 g, 2.39 mmol), DIEA (0.222 g, 3.8883 mmol), TATU (1.300 g, 3.41 mmol), DMF (5 mL). The reaction mixture was stirred at room temperature for 16 h. The reaction mixture was filtered through celite pad and celite pad w as washed with ACN (3×20 mL). The filtrate was then concentrated under reduced pressure to afford the crude product. The reaction mixture was purified by C18 column eluted with ACN/H₂O (v/v=1/3), filtered and concentrated under vacuum. This resulted in 0.446 g, (106.37% yield) of 3-methoxy-N-(2-methoxyethyl)-4-(prop-2-yn-1-ylamino)benzamide as brown oil. LCMS: m/z=263 [M+1]⁺.

### Step 2. 4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-methoxy-N-(2-methoxyethyl)benzamide (85)

Into a 10-mL round-bottom flask was placed 3-methoxy-N-(2-methoxyethyl)-4-(prop-2-yn-1-ylamino)benzamide (0.278 g, 1.05 mmol), (Z)-3-fluoro-N-(2-iodo-3-(2,2,2-trifluoroethyl)benzo[b]thiophen-7-yl)-1-methylpiperidin-4-amine (0.082 g, 173.62 μmol), Pd(PPh₃)₂Cl₂ (0.030 g, 42.49 μmol), CuI (0.025 g, 131.26 μmol), DIEA (0.089 g, 688.62 μmol), methyl sulfoxide (2 mL). The reaction was stirred under nitrogen atmosphere at RT for 16 h. The reaction mixture was filtered through celite pad and celite pad was washed with ACN (3×20 mL). The filterate was then concentrated under reduced pressure to afford the crude product. The crude product was purified by preparative HPLC (Mobile Phase A: water (0.1% ammonium hydroxide), Mobile Phase B: ACN; Flow rate: 70 mL/min; Gradient: 50-85-100% B (2-30-60 min); 266 nm; RT: 33.158-34.443) to provide the desired product. This resulted in 0.039 g (37.02% yield) of 4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-methoxy-N-(2-methoxyethyl)benzamide (85) as white solid. LCMS: m/z=607 [M+1]⁺.

¹H NMR (400 MHz, DMSO-d₆) δ 8.12 (t, J=5.3 Hz, 1H), 7.38-7.36 (m, 1H), 7.29 (d, J=1.8 Hz, 1H), 7.22-7.09 (m, 2H), 6.68 (t, J=7.3 Hz, 2H), 5.94 (t, J=6.4 Hz, 1H), 5.06 (d, J=8.5 Hz, 1H), 4.71 (d, J=49.5 Hz, 1H), 4.26 (d, J=5.9 Hz, 2H), 3.76 (s, 3H), 3.73 (d, J=11.0 Hz, 1H), 3.35 (t, J=5.2 Hz, 2H), 3.33-3.29 (m, 2H), 3.21 (s, 2H), 3.18 (s, 3H), 2.94 (t, J=11.5 Hz, 1H), 2.71 (d, J=10.9 Hz, 1H), 2.18 (d, J=13.0 Hz, 1H), 2.10 (s, 3H), 1.99 (t, J=11.4 Hz, 1H), 1.95-1.81 (m, 1H), 1.68-1.58 (m, 1H).

### Example 86

### (4-((3-(7-(((Z)-3-fluoro-1-isopropylpiperidin-4-yl)amino)-3-(2,2,2-trifluoroethyl)benzo[b]thiophen-2-yl)prop-2-yn-1-yl)amino)-3-methoxyphenyl)dimethylphosphine oxide(racemic) (86)

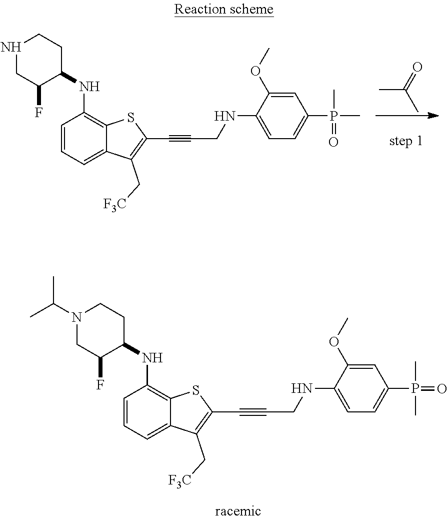

### Experimental Details

### Step 1. (4-((3-(7-(((Z)-3-fluoro-1-isopropylpiperidin-4-yl)amino)-3-(2,2,2-trifluoroethyl)benzo[b]thiophen-2-yl)prop-2-yn-1-yl)amino)-3-methoxyphenyl)dimethylphosphine oxide(racemic) (86)

Into a 4-mL sealed tube purged and maintained with an inert atmosphere of nitrogen, was placed sodium cyanoboronhydride (0.041 g, 956.25 μmol), acetic acid (0.1 mL), (4-((3-(7-(((Z)-3-fluoropiperidin-4-yl)amino)-3-(2,2,2-trifluoroethyl)benzo[b]thiophen-2-yl)prop-2-yn-1-yl)amino)-3-methoxyphenyl)dimethylphosphine oxide (0.062 g, 109.24 μmol), acetone (1 mL). The reaction mixture was stirred at 50° C. for 15 h. The reaction was quenched by the addition of water (5 mL) and extracted with EA (2×5 mL). The organic layer was combined, washed with brine (5 mL), dried over anhydrous Na₂SO₄ and concentrated under vacuum. The mixture was purified by preparative HPLC (Mobile Phase A: water (10 mmoL/L ammonium hydroxide), Mobile Phase B: ACN; Flow rate: 70 mL/min; Gradient: 35-65-85% B (2-30-60 min; 266 nm; RT: 34.273-36.349 min). This resulted in 0.043 g (64.57% yield) of (4-((3-(7-(((Z)-3-fluoro-1-isopropylpiperidin-4-yl)amino)-3-(2,2,2-trifluoroethyl)benzo[b]thiophen-2-yl)prop-2-yn-1-yl)amino)-3-methoxyphenyl)dimethylphosphine oxide(racemic) (86) as white solid. LCMS: m/z=610 [M+1]⁺.

¹H NMR (400 MHz, DMSO) δ 7.24 (d, J=8.0 Hz, 1H), 7.19 (s, 1H), 7.17 (s, 1H), 7.13 (s, 1H), 7.10 (s, 1H), 6.83 (s, 1H), 6.76 (d, J=7.4 Hz, 1H), 6.03 (s, 1H), 4.32 (d, J=5.5 Hz, 3H), 3.83 (s, 5H), 1.58 (s, 5H), 1.54 (s, 5H), 0.97-0.92 (m, 10H).

### Example 87

(Z)-N-[2-[3-(4-dimethylphosphoryl-2-methoxy-anilino)prop-1-ynyl]-3-(2,2,2-trifluoroethyl)benzothiophen-7-yl]-3-fluoro-1-(trifluoromethyl)piperidin-4- amine (87)

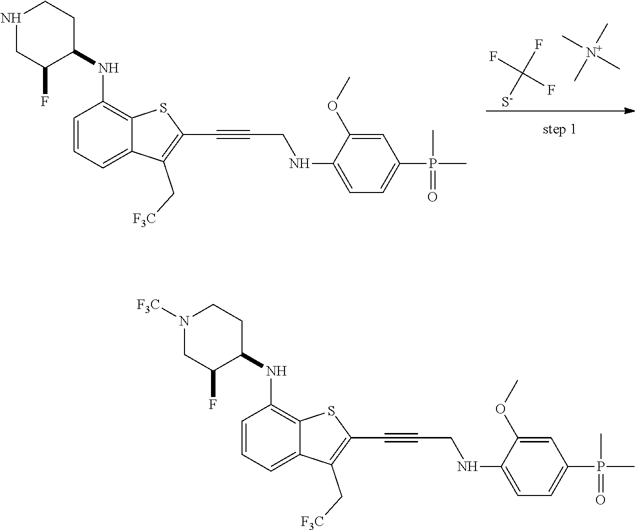

### Experiment Details

### Step 1. Synthesis of (Z)—N-[2-[3-(4-dimethylphosphoryl-2-methoxy-anilino)prop-1-ynyl]-3-(2,2,2-trifluoroethyl)benzothiophen-7-yl]-3-fluoro-1-(trifluoromethyl)piperidin-4-amine (87)

Into a 4-mL vial was placed (4-((3-(7-(((Z)-3-fluoropiperidin-4-yl)amino)-3-(2,2,2-trifluoroethyl)benzo[b]thiophen-2-yl)prop-2-yn-1-yl)amino)-3-methoxyphenyl)dimethylphosphine oxide (0.089 g, 156.81 μmol), MeCN (1 mL), tetramethylammonium trifluoromethanethiolate (0.072 g, 410.92 μmol). The mixture was stirred at 50° C. overnight. The resulted reaction was purified by C₁₈ chromatography column eluted with ACN/water (0.1% ammonium bicarbonate) (v/v=1/1) to give the product. This resulted in 0.100 g (16.96% yield) of (Z)—N-[2-[3-(4-dimethylphosphoryl-2-methoxy-anilino)prop-1-ynyl]-3-(2,2,2-trifluoroethyl)benzothiophen-7-yl]-3-fluoro-1-(trifluoromethyl)piperidin-4-amine (87) as white solid. LCMS: m/z=636 [M+1]⁺.

¹H NMR (400 MHz, DMSO-d6) δ 7.27 (q, J=8.7, 8.3 Hz, 1H), 7.21 (ddd, J=11.6, 7.1, 2.2 Hz, 2H), 7.17-7.06 (m, 1H), 6.88-6.79 (m, 2H), 6.06 (s, 1H), 5.39 (d, J=8.4 Hz, 1H), 4.97 (s, 1H), 4.85 (s, OH), 4.34 (d, J=5.1 Hz, 2H), 3.84 (s, 1H), 3.87-3.73 (m, 5H), 3.51 (dd, J=13.0, 10.1 Hz, 1H), 3.04 (d, J=13.3 Hz, 1H), 2.95 (d, J=13.2 Hz, 1H), 2.84-2.74 (m, 1H), 2.06-1.92 (m, 1H), 1.83 (d, J=12.9 Hz, 1H), 1.58 (d, J=13.2 Hz, 6H).

### Example 88

### (4-((3-(7-(((Z)-3-fluoro-1-(2-methoxyethyl)piperidin-4-yl)amino)-3-(2,2,2-trifluoroethyl)benzo[b]thiophen-2-yl) prop-2-yn-1-yl)amino)-3-methoxyphenyl)dimethylphosphine oxide (racemic) (88)

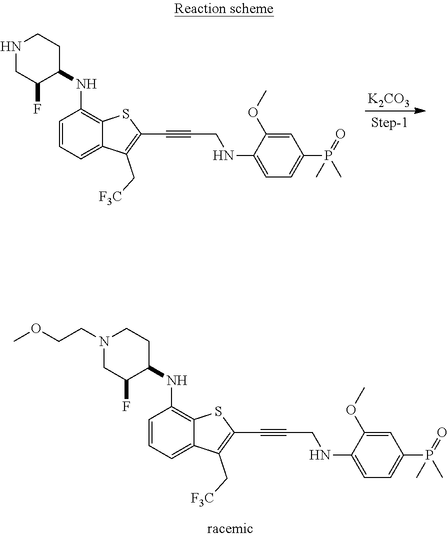

### Experimental Details

### Step 1. (4-((3-(7-(((Z)-3-fluoro-1-(2-methoxyethyl)piperidin-4-yl)amino)-3-(2,2,2-trifluoroethyl)benzo[b]thiophen-2-yl)prop-2-yn-1-yl)amino)-3-methoxyphenyl)dimethylphosphine oxide (racemic) (88)

Into a 100-mL three necked bottle purged and maintained with an inert atmosphere of nitrogen, was placed (4-((3-(7-(((Z)-3-fluoropiperidin-4-yl)amino)-3-(2,2,2-trifluoroethyl)benzo[b]thiophen-2-yl)prop-2-yn-1-yl)amino)-3-methoxyphenyl)dimethylphosphine oxide (racemic) (0.111 g, 0.19 mmol), 1-bromo-2-methoxyethane (0.134 g, 0.94 mmol), K₂CO₃ (0.123 g, 0.89 mmol), DMA (2 mL). The reaction mixture was stirred at 50C for 16 h. The resulting solution was added to water (30 mL). The resulting solution was extracted with EA (2×15 mL). The organic layers combined and washed with brine (20 mL), dried over anhydrous Na₂SO₄, the residue was concentrated under vacuum. The mixture was purified by preparative HPLC (Mobile Phase A: water (10 mmoL/L ammonium hydroxide), Mobile Phase B: ACN; Flow rate: 40 mL/min; Gradient: 40-65-65% B (2-30-60 min); 266 nm; RT: 24.424-26.063 min). This resulted in 0.049 g (40.05% yield) of (4-((3-(7-(((Z)-3-fluoro-1-(2-methoxyethyl)piperidin-4-yl)amino)-3-(2,2,2-trifluoroethyl)benzo[b]thiophen-2-yl)prop-2-yn-1-yl)amino)-3-methoxyphenyl)dimethylphosphine oxide (racemic) (88) as off white solid. LCMS: m/z=626 [M+1]⁺.

¹H NMR (400 MHz, DMSO) δ 7.24-7.00 (m, 4H), 6.76 (d, J=5.6 Hz, 1H), 6.69 (d, J=7.6 Hz, 1H), 5.96 (t, J=6.1 Hz, 1H), 5.03 (d, J=8.5 Hz, 1H), 4.70 (d, J=49.4 Hz, 1H), 4.25 (d, J=5.7 Hz, 2H), 3.76 (s, 3H), 3.74-3.65 (m, 2H), 3.65-3.47 (m, 1H), 3.34 (t, J=5.7 Hz, 2H), 3.15 (s, 3H), 3.05 (t, J=10.4 Hz, 1H), 2.81 (d, J=10.9 Hz, 1H), 2.45 (s, 1H), 2.32 (d, J=13.0 Hz, 1H), 2.23 (d, J=13.1 Hz, 1H), 2.12 (t, J=11.0 Hz, 1H), 1.89-1.76 (m, 1H), 1.63 (d, J=10.3 Hz, 1H), 1.51 (s, 3H), 1.48 (s, 3H).

### Example 89

### (3-methoxy-4-((3-(7-((5-methyl-5-azaspiro[2.5]octan-8-yl)amino)-3-(2,2,2-trifluoroethyl)benzo[b]thiophen-2-yl)prop-2-yn-1-yl)amino)phenyl)dimethylphosphine oxide (89)

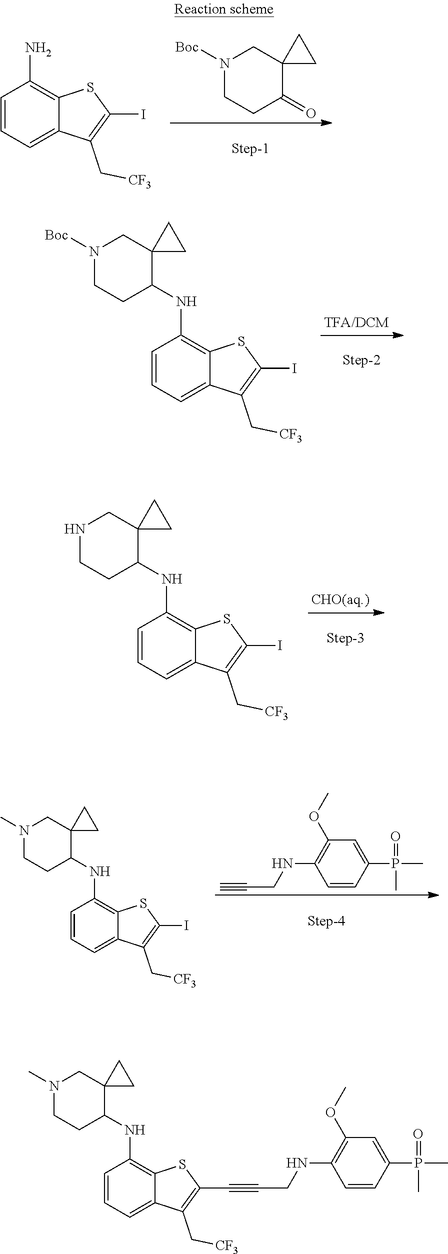

### Experiment Details

### Step 1. tert-butyl 3-fluoro-4-(3-methoxy-4-(prop-2-yn-1-ylamino)benzamido)piperidine-1-carboxylate

Into an ice-cold 8 mL vail was placed 2-iodo-3-(2,2,2-trifluoroethyl)benzo[b]thiophen-7-amine (0.504 g, 1.41 mmol), tert-butyl 8-oxo-5-azaspiro[2.5]octane-5-carboxylate (0.662 g, 2.94 mmol), TMS-C1 (1.516 g, 13.95 mmol), DMF (3 mL). The reaction was stirred for 1 h at 0-5-carbox₃-THF (3 mL) was added. The reaction was stirred for an additional 2 h. LCMS showed the reaction was complete. The reaction was quenched with MeOH (10 mL), and concentrated with vacuum. The residue was purified with Silica-gel column, eluted with n-Heptane/EA (v/v=1/3) to afford tert-butyl 3-fluoro-4-(3-methoxy-4-(prop-2-yn-1-ylamino)benzamido)piperidine-1-carboxylate (0.316 g, 557.89 μmol, 39.53% yield) as off-white solid. LCMS: m/z=567 [M+1]⁺.

### Step 2. N-(2-iodo-3-(2,2,2-trifluoroethyl)benzo[b]thiophen-7-yl)-5-azaspiro[2.5]octan-8-amine

Into a 25 mL flask was placed tert-butyl 3-fluoro-4-(3-methoxy-4-(prop-2-yn-1-ylamino)benzamido)piperidine-1-carboxylate (0.312 g, 550.83 μmol), TFA (0.2 mL), DCM (1 mL). The reaction was stirred 0.5 h at room temperature. LCMS showed the reaction was complete. The reaction quenched with saturated NaHCO₃ aq., The solution was diluted with water (5 mL), extracted with EA (2 mL×2). The combined organic lay ers were washed with water (2 mL) and brine (2 mL) successively, separated, then concentrated with vacuum to afford N-(2-iodo-3-(2,2,2-trifluoroethyl)benzo[b]thiophen-7-yl)-5-azaspiro[2.5]octan-8-amine (0.215 g, 461.07 μmol, 83.71% yield) as an off-white solid. LCMS: m/z=467[M+1]⁺

### Step 3. N-(2-iodo-3-(2,2,2-trifluoroethyl)benzo[b]thiophen-7-yl)-5-methyl-5-azaspiro[2.5]octan-8-amine

Into a 4 mL vail was placed N-(2-iodo-3-(2,2,2-trifluoroethyl)benzo[b]thiophen-7-yl)-5-azaspiro[2.5]octan-8-amine (0.204 g, 437.48 μmol), formaldehyde (0.059 g, 687.74 μmol), sodium cyanoboronhydride (0.121 g, 2.82 mmol), MeOH (1 mL), acetic acid (0.01 mL). The reaction was stirred for 1.5 h at room temperature. LCMS showed the reaction was complete. The residue was purified with Silica-gel column, eluted with Me OH/DCM (v/v=1/10) to afford N-(2-iodo-3-(2,2,2-trifluoroethyl)benzo[b]thiophen-7-yl)-5-methyl-5-azaspiro[2.5]octan-8-amine (0.096 g, 199.86 μmol, 45.68% yield) as an light yellow oil. LCMS: m/z=481[M−1]⁻

### Step 4. (3-methoxy-4-((3-(7-((5-methyl-5-azaspiro[2.5]octan-8-yl)amino)-3-(2,2,2-trifluoroethyl)benzo[b]thiophen-2-yl)prop-2-yn-1-yl)amino)phenyl)dimethylphosphine oxide (89)

Into a 25 mL flask purged and maintained with nitrogen atmosphere was placed N-(2-iodo-3-(2,2,2-trifluoro ethyl)benzo[b]thiophen-7-yl)-5-methyl-5-azaspiro[2.5]octan-8-amine (0.096 g, 199.86 μmol), (3-methoxy-4-(prop-2-yn-1-ylamino)phenyl)dimethylphosphine oxide (0.162 g, 682.87 μmol), Pd(PPh₃)₂Cl₂ (0.041 g, 58.08 μmol), CuI (0.019 g, 99.76 μmol), TEA (0.124 g, 1.23 mmol), DMF (1 mL), and stirred for 3 h at room temperature. LCMS monitored that the reaction was complete. The reaction was quenched with water (4 mL), extracted with EA (3 mL×2). The combined organic layers were washed with water (4 mL) and brine (4 mL) successively, separated, then concentrated with vacuum. The residue was pre-purified with Silica-gel column, eluted with DCM/MeOH (v/v=3/1), then re-purified with C₁₈ column, eluted with ACN/water (v/v=1:1) to afford (3-methoxy-4-((3-(7-((5-methyl-5-azaspiro[2.5]octan-8-yl)amino)-3-(2,2,2-trifluoroethyl)benzo[b]thiophen-2-yl)prop-2-yn-1-yl)amino)phenyl)dimethylphosphine oxide (89) (0.020 g, 33.92 μmol, 16.97% yield) as a white solid. LCMS: m/z=590 [M+1]⁺.

¹H NMR (400 MHz, DMSO-d₆) δ 7.31-7.08 (m, 4H), 6.92-6.80 (m, 1H), 6.67 (d, J=7.8 Hz, 1H), 6.05 (t, J=6.3 Hz, 1H), 4.89 (d, J=7.8 Hz, 1H), 4.33 (d, J=6.3 Hz, 2H), 3.80 (d, J=36.1 Hz, 5H), 3.51 (s, 1H), 2.30-2.11 (m, 5H), 1.81 (d, J=57.5 Hz, 4H), 1.58 (d, J=13.2 Hz, 6H), 0.65 (s, 1H), 0.25 (d, J=44.1 Hz, 3H).

### Example 90

### 4-((2-(3-((4-(dimethylphosphoryl)-2-methoxyphenyl)amino)prop-1-yn-1-yl)-3-(2,2,2-trifluoroethyl)benzo[b]thiophen-7-yl)amino)-1-methylpiperidine-3-carbonitrile (90)

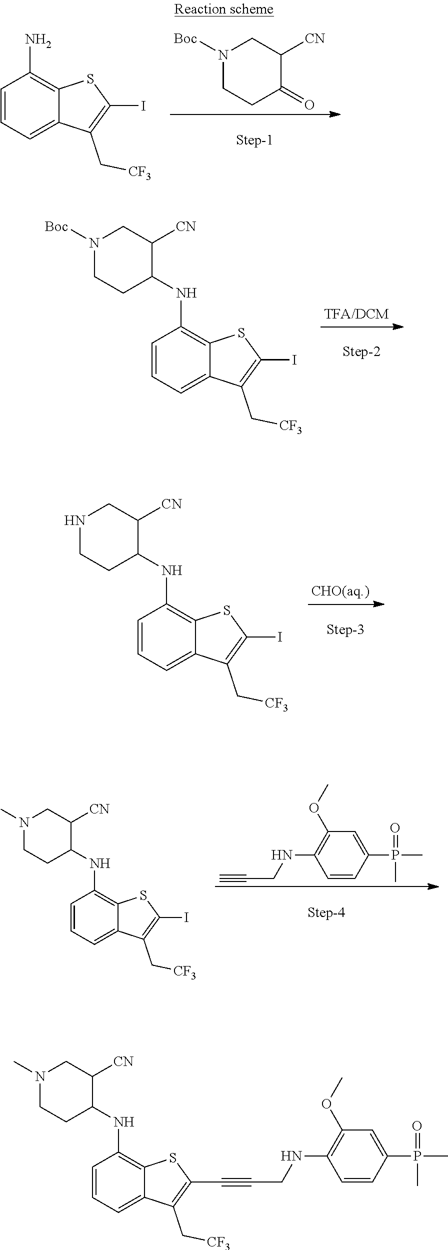

### Experiment Details

### Step 1. tert-butyl 3-cyano-4-((2-iodo-3-(2,2,2-trifluoroethyl)benzo[b]thiophen-7-yl)amino)piperidine-1-carboxylate

Into an opened 40 mL vail was placed tert-butyl 3-cyano-4-oxopiperidine-1-carboxylate (1.264 g, 5.64 mmol), 2-iodo-3-(2,2,2-trifluoroethyl)benzo[b]thiophen-7-amine (1.004 g, 2.81 mmol). The reaction was heated to 150° C. and stirred for 30 min then cooled to room temperature naturally. LCMS monitored intermediate w as fully converted. The reaction was purified with silica-gel column, eluted with DCM/MeOH (v/v=10/1).

Into a ice-cold 40 mL vail was placed the pure intermediate, BH₃-THF (3 mL), DCM (5 mL). The reaction was stirred for 40 min at room temperature. LCMS monitored the reaction was complete. The reaction was quenched by MeOH, purified with silica-gel column (DCM/MeOH v/v=10/1) to afford tert-butyl 3-cyano-4-((2-iodo-3-(2,2,2-trifluoroethyl)benzo[b]thiophen-7-yl)amino)piperidine-1-carboxylate (0.153 g, 270.61 μm ol, 9.63% yield) as off-white solid. LCMS: m/z=566[M+1]⁺.

### Step 2. 4-((2-iodo-3-(2,2,2-trifluoroethyl)benzo[b]thiophen-7-yl)amino)piperidine-3-carbonitrile

Into a 4 mL flask was placed tert-butyl 3-cyano-4-((2-iodo-3-(2,2,2-trifluoroethyl)benzo[b]thiophen-7-yl)amino) piperidine-1-carboxylate (0.149 g, 263.53 μmol), TFA (0.2 mL), DCM (1 mL). The reaction was stirred for 1 h at room temperature. LCMS showed the reaction was complete. The reaction quenched with saturated NaHCO₃ aq., The solution was diluted with water (2 mL), extracted with EA (2 mL×2). The combine d organic layers were washed with water (2 mL) and brine (2 mL) successively, separated, then concentrated with vacuum to afford crude 4-((2-iodo-3-(2,2,2-trifluoroethyl)benzo[b]thiophen-7-yl)amino)piperidine-3-carbonitrile (0.153 g, 328.84 μmol, 124.78% yield) as an off-white solid. LCMS: m/z=466[M+1]⁺

### Step 3. 4-((2-iodo-3-(2,2,2-trifluoroethyl)benzo[b]thiophen-7-yl)amino)-1-methylpiperidine-3-carbonitrile

Into a 4 mL vail was placed 4-((2-iodo-3-(2,2,2-trifluoroethyl)benzo[b]thiophen-7-yl)amino)piperidine-3-carbo nitrile (0.058 g, 124.66 μmol), formaldehyde (0.032 g, 351.70 μmol), sodium cyanoboronhydride (0.057 g, 1.33 mmol), MeOH (1 mL), acetic acid (0.01 mL). The reaction was stirred for 1.5 h at room temperature. LCMS showed the reaction was complete. The residue was purified with C₁₈ column, eluted with ACN/w ater (v/v=2/3) to afford 4-((2-iodo-3-(2,2,2-trifluoroethyl)benzo[b]thiophen-7-yl)amino)-1-methylpiperidine-3-carbonitrile (0.038 g, 79.28 μmol, 63.60% yield) as an light yellow oil. LCMS: m/z=480[M−1]⁻

### Step 4. 4-((2-(3-((4-(dimethylphosphoryl)-2-methoxyphenyl)amino)prop-1-yn-1-yl)-3-(2,2,2-trifluoroethyl)benzo[b]thiophen-7-yl)amino)-1-methylpiperidine-3-carbonitrile (90)

Into a 4 mL vial purged and maintained with nitrogen atmosphere was placed 4-((2-iodo-3-(2,2,2-trifluoroethyl)benzo[b]thiophen-7-yl)amino)-1-methylpiperidine-3-carbonitrile (0.032 g, 68.78 μmol), (3-methoxy-4-(prop-2-yn-1-ylamino)phenyl)dimethylphosphine oxide (0.036 g, 151.75 μmol), Pd(PPh₃)₂Cl₂ (0.012 g, 17.00 μmol), CuI (0.004 g, 21.00 μmol), TEA (0.027 g, 266.83 μmol), DMF (1 mL), and stirred for 1.5 h at room temperature. LCMS monitored that the reaction was complete. The reaction was quenched with water (2 mL), extracted with EA (2 mL×2). The combined organic layers were washed with water (2 mL) and brine (2 mL) successively, separated, then concentrated with vacuum. The residue was purified with prep-HPLC (Mobile Phase A: water (formic acid), Mobile Phase B: ACN; Flow rate: 40 mL/min; Gradient: 40-70-95% B (2-30-60 min); 266 nm; RT: 32.353-34.117 min) to afford 4-((2-(3-((4-(dimethylphosphoryl)-2-methoxyphenyl)amino)prop-1-yn-1-yl)-3-(2,2,2-trifluoroethyl)benzo[b]thiophen-7-yl)amino)-1-methylpiperidine-3-carbonitrile (90) (0.017 g, 28.88 μmol, 41.99% yield) as a white solid. LCMS: m/z=589 [M+1]⁺.

¹H NMR (400 MHz, DMSO-d₆) δ 7.32-7.11 (m, 4H), 6.88-6.72 (m, 2H), 6.06 (t, J=6.3 Hz, 1H), 5. 66 (d, J=6.5 Hz, 1H), 4.34 (d, J=6.3 Hz, 2H), 3.83 (d, J=16.2 Hz, 5H), 3.62 (s, 1H), 3.50 (s, 1H), 2.98-2.83 (m, 2H), 2.22 (s, 4H), 2.11-1.91 (m, 2H), 1.82 (d, J=11.7 Hz, 1H), 1.58 (d, J=13.1 Hz, 6H).

### Example 91

(2,5-difluoro-4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)phenyl)dimethylphosphine oxide (racemic) (91)

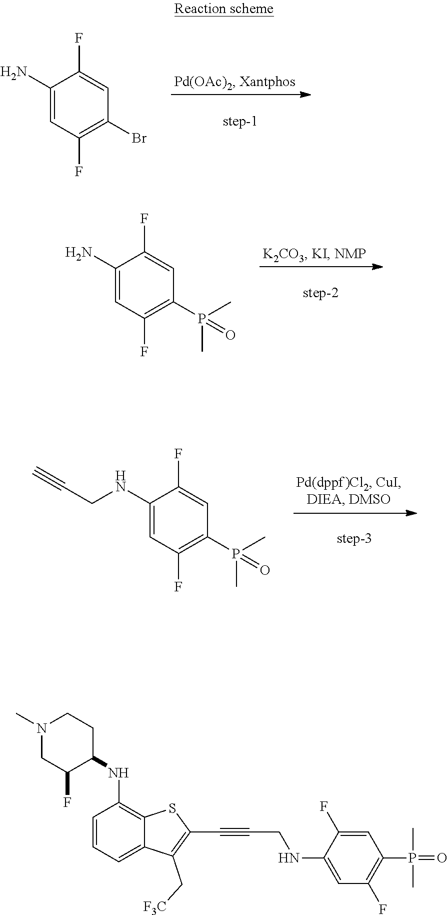 [SMILES: Nc1cc(F)c(Br)cc1F ; CP(C)(=O)c1cc(F)c(N)cc1F ; C#CCNc1cc(F)c(P(C)(C)=O)cc1F ; CN1CC[C@@H](Nc2cccc3c(CC(F)(F)F)c(C#CCNc4cc(F)c(P(C)(C)=O)cc4F)sc23)[C@@H](F)C1]

### Experiment Details

### Step 1. (4-amino-2,5-difluorophenyl)dimethylphosphine oxide

Into a 20-mL sealed tube purged and maintained with an inert atmosphere of nitrogen, was placed was placed 4-bromo-2,5-difluoroaniline (1.16 g, 5.58 mmol), Pd(OAc)₂ (0.25 g, 1.11 mmol), Xantphos (0.67 g, 1.16 mmol), DIEA (1.93 g, 14.93 mmol), DMF (10 mL). The reaction mixture was stirred at 130° C. for 10 mi n, Methylphosphinoylmethane (1.03 g, 13.20 mmol) was added. The mixture was stirred at 130° C. for 30 min. The mixture was purified with C18 column eluted with ACN/water (v/v=1/4) to afford 1.238 g (crude) of (4-amino-2,5-difluorophenyl)dimethylphosphine oxide as little-yellow solid. LCMS: m/z=206 [M+1]⁺

### Step 2. (2,5-difluoro-4-(prop-2-yn-1-ylamino)phenyl)dimethylphosphine oxide

Into a 20-mL sealed was placed (4-amino-2,5-difluorophenyl)dimethylphosphine oxide (1.157 g, 5.64 mmol), 3-Bromopropyne (1.007 g, 8.47 mmol), Cs₂CO₃ (1.383 g, 10.01 mmol), KI (1.047 g, 6.31 mmol), DMF (10 mL). The reaction mixture was stirred at 80° C. for 12 h. The mixture was purified with C18 column eluted with ACN/water (v/v=1/4) to afford 0.063 g (4.59%) of (2,5-difluoro-4-(prop-2-yn-1-ylamino)phenyl)dimethylphosphine oxide as yellow oil. LCMS: m/z=244 [M+1]⁺.

### Step 3. (2,5-difluoro-4-((3-(7-(((Z)-3-fluoro-1-methylpiperidin-4-yl)amino)-3-(2,2,2-trifluoroethyl)benzo[b]thio phen-2-yl)prop-2-yn-1-yl)amino)phenyl)dimethylphosphine oxide (racemic) (91)

Into a 8-mL sealed tube purged and maintained with an inert atmosphere of nitrogen, was placed (Z)-3-fluoro-N-(2-iodo-3-(2,2,2-trifluoroethyl)benzo[b]thiophen-7-yl)-1-methylpiperidin-4-amine (racemic) (0.066 g, 0.14 mmol), (2,5-difluoro-4-(prop-2-yn-1-ylamino)phenyl)dimethylphosphine oxide (0.063 g, 0.26 mmol), Pd(dppf) Cl₂ (0.016 g, 0.02 mmol), CuI (0.017 g, 0.09 mmol), DIEA (0.060 g, 0.46 mmol), DMSO (1 mL). The reaction mixture was stirred at 50° C. for 1 h. The mixture was purified by preparative HPLC (Mobile Phase A: water (10 mmoL/L ammonium hydroxide), Mobile Phase B: MeCN; Flow rate: 40 mL/min; Gradient: 25-55-55% B (2-30-60 min); 250 nm; RT: 28.975-30.590 min) to afford (2,5-difluoro-4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)phenyl)dimeth ylphosphine oxide (racemic) (91) (0.025 g, 26.52% yield) as white solid. LCMS: m/z=564 [M+1]⁺

¹H NMR (400 MHz, CD₃OD) δ 7.38-7.27 (m, 2H), 7.21 (d, J=8.0 Hz, 1H), 6.86-6.75 (m, 2H), 4.88 (d, J=13.2 Hz, 1H), 4.38 (s, 2H), 3.79-3.65 (m, 3H), 3.25-3.11 (m, 1H), 2.98-2.84 (m, 1H), 2.40 (d, J=13.2 Hz, 1H), 2.30 (d, J=8.0 Hz, 3H), 2.28-2.17 (m, 1H), 2.03-1.90 (m, 2H), 1.79 (s, 3H), 1.76 (s, 3H).

### Example 92

### (5-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-4-methoxypyridin-2-yl)dimethylphosphine oxide (92)

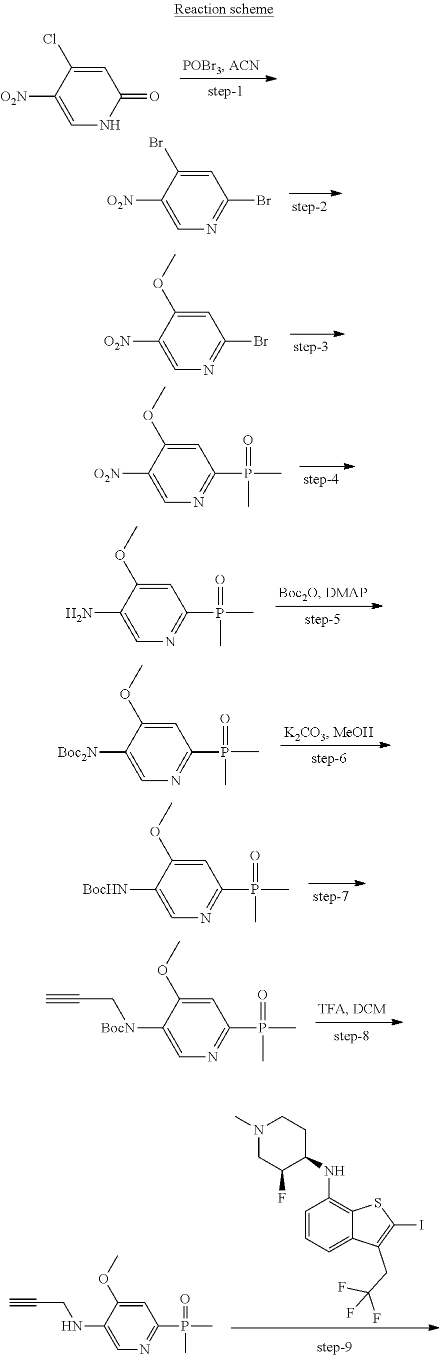

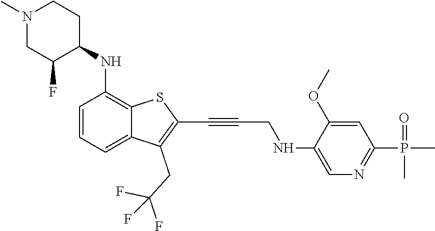

### Experimental Details

### Step 1. 2,4-dibromo-5-nitropyridine

Into a 40-mL reaction vial purged and maintained with an inert atmosphere of nitrogen, was placed 4-chloro-5-nitropyridin-2(1H)-one (1.85 g, 10.61 mmol), POBr₃ (3.50 g, 12.21 mmol), MeCN (20 mL). The reaction mixture was stirred at 0° C. for 3 h. The resulting solution was concentrated under vacuum and poured into water (50 mL). The resulting solution was extracted with EA (2×100 mL). The organic layers combined and washed with brine (50 mL), dried over anhydrous Na₂SO₄, the residue was concentrated under vacuum. The residue was applied onto a silica gel column eluted with EA:hexane (v:v=1:1). This resulted in 2.20 g (73.51% yield) of 2,4-dibromo-5-nitropyridine as yellow solid. LCMS: m/z=281 [M+1]⁺

### Step 2. 2-bromo-4-methoxy-5-nitropyridine

Into a 100-mL three necked bottle purged and maintained with an inert atmosphere of nitrogen, was placed 2,4-dibromo-5-nitropyridine (1.90 g, 6.74 mmol), DMSO (15 mL), MeOH (10 mL) and stirred at 0° C., then added dropwise sodium methanolate (1.07 g, 19.91 mmol) in MeOH (10 mL). The mixture was stirred at 50° C. for 4 h. The resulting solution was quenched with water (60 mL). The resulting solution was extracted with EA (2×100 mL). The organic layers combined and washed with brine (50 mL), dried over an hydrous Na₂SO₄, the residue was concentrated under vacuum. The residue was applied onto a silica gel column eluted with ACN/H₂O (0.01% TFA) (v:v=1:1), Flow rate: 100 m/min. This resulted in 1.10 g (69.96% yield) of 2-bromo-4-methoxy-5-nitropyridine as yellow solid. LCMS: m/z=233 [M+1]⁺

### Step 3. (4-methoxy-5-nitropyridin-2-yl)dimethylphosphine oxide

Into a 40-mL reaction vial purged and maintained with an inert atmosphere of nitrogen, was placed 2-bromo-4-methoxy-5-nitropyridine (1.10 g, 4.71 mmol), Pd(OAc)₂ (235.00 mg, 1.05 mmol), Xantphos (671.00 mg, 1.16 mmol), DIEA (1.46 g, 11.35 mmol), DMF (15 ML) and stirred at 110° C. for 5 min, then added dropwise Phosphine oxide (1.22 g, 15.73 mmol) in DMF (5 mL). The reaction mixture was stirred at 110° C. for 2 h. The resulting solution was concentrated under vacuum. The residue was applied onto a C₁₈ column eluted with ACN/H₂O (0.01% TFA) (v:v=1:1), Flow rate: 100 mL/min. This resulted in 1.00 g (92.12% yield) of (4-methoxy-5-nitropyridin-2-yl)dimethylphosphine oxide as yellow solid. LCMS: m/z=231 [M+1]⁺

### Step 4. (5-amino-4-methoxypyridin-2-yl)dimethylphosphine oxide

Into a 100-mL three necked bottle purged and maintained with an inert atmosphere of nitrogen, was placed (4-methoxy-5-nitropyridin-2-yl)dimethylphosphine oxide (1.05 g, 4.56 mmol), Fe (839 mg, 15.02 mmol), NH₄Cl (1.08 g, 20.26 mmol), ETOH (10 mL), H₂O (2 mL). The reaction mixture was stirred at 80° C. for 2 h. The reaction mixture was filtered and the filter cake was washed with EtOH (20 mL). The residue was concentrated under vacuum. The residue was applied onto a C₁₈ column eluted with ACN/H₂O (0.01% TFA) (v:v=1:1), Flow rate: 100 mL/min. This resulted in 857.00 mg (93.84% yield) of (5-amino-4-methoxypyridin-2-yl)dimethylphosphine oxide as yellow solid. LCMS: m/z=201 [M+1]⁺

### Step 5. tert-butyl N-tert-butoxycarbonyl-N-(6-dimethylphosphoryl-4-methoxy-3-pyridyl)carbamate

Into a 40-mL reaction vial purged and maintained with an inert atmosphere of nitrogen, was placed (5-amino-4-methoxypyridin-2-yl)dimethylphosphine oxide (811.00 mg, 4.05 mmol), DCM (10 mL), Di-tert-butyl di carbonate (3.04 g, 13.96 mmol), DMAP (320.00 mg, 2.62 mmol), DIEA (3.17 g, 24.52 mmol). The reaction mixture was stirred at room temperature for 12 h. The reaction mixture concentrated under vacuum. The residue was applied onto a silica gel column eluted with DCM/MeOH (v:v=10:1). This resulted in 850.00 mg (53.39% yield) of tert-butyl N-tert-butoxycarbonyl-N-(6-dimethylphosphoryl-4-methoxy-3-pyridyl)carbamate. LCMS: m/z=401 [M+1]⁺

### Step 6. tert-butyl (6-(dimethylphosphoryl)-4-methoxypyridin-3-yl)carbamate

Into a 100-mL three necked bottle purged and maintained with an inert atmosphere of nitrogen, was placed tert-butyl N-tert-butoxycarbonyl-N-(6-dimethylphosphoryl-4-methoxy-3-pyridyl)carbamate (800.00 mg, 1.99 mmol), K₂CO₃ (1.02 g, 7.43 mmol), MeOH (10 mL). The reaction mixture was stirred at 50° C. for 2 h. The reaction mixture was filtered and the filter cake was washed with ACN (10 mL). The residue was concentrated under vacuum. This resulted in 857.00 mg (93.84% yield) of tert-butyl (6-(dimethylphosphoryl)-4-methoxypyridin-3-yl)carbamate as yellow solid. LCMS: m/z=301 [M+1]⁺

### Step 7. tert-butyl (6-(dimethylphosphoryl)-4-methoxypyridin-3-yl)(prop-2-yn-1-yl)carbamate

Into a 100-mL three necked bottle purged and maintained with an inert atmosphere of nitrogen, was placed tert-butyl (6-(dimethylphosphoryl)-4-methoxypyridin-3-yl)carbamate (410.00 mg, 1.36 mmol), DMF (10 mL) The reaction mixture was cooled to 0° C. NaH (175.00 mg, 4.37 mmol) was added and stirred at 10° C.˜20° C. for 30 minutes. 3-bromoprop-1-yne (725.00 mg, 6.09 mmol) in DMF (3 mL) was added to the reaction at 0° C. The reaction mixture was stirred at room temperature for 1 h. The resulting solution was quenched with H₂O (50 mL) and extracted with EA (2×60 mL). The organic layers combined and washed with brine (100 mL), dried over anhydrous Na₂SO₄, the residue was concentrated under vacuum. The residue was applied onto a silica gel column eluted with EA:hexane (v:v=1:1). This resulted in 450.00 mg (97.41% yield) of tert-butyl (6-(dimethylphosphoryl)-4-methoxypyridin-3-yl)(prop-2-yn-1-yl)carbamate as brow n solid. LCMS: m/z=339 [M+1]⁺

### Step 8. (4-methoxy-5-(prop-2-yn-1-ylamino)pyridin-2-yl)dimethylphosphine oxide

Into a 50-mL three necked bottle purged and maintained with an inert atmosphere of nitrogen, was placed tert-butyl (6-(dimethylphosphoryl)-4-methoxypyridin-3-yl)(prop-2-yn-1-yl)carbamate (400.00 g, 1.18 mmol), T FA (2 mL) and DCM (10 mL). The reaction mixture was stirred at room temperature for 3 h. The resulting solution was concentrated under vacuum. The residue was applied onto a C₁₈ column eluted ACN/H₂O (0.01% TFA) (v:v=1:1). This resulted in 300.00 mg (72.03% yield) of (4-methoxy-5-(prop-2-yn-1-ylamino)pyridin-2-yl)dimethylphosphine oxide as yellow oil. LCMS: m/z=239 [M+1]

### Step 9. (5-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-4-methoxypyridin-2-yl)dimethylphosphine oxide (92)

Into a 40-mL three necked bottle purged and maintained with an inert atmosphere of nitrogen, was placed (3S,4R)-3-fluoro-N-(2-iodo-3-(2,2,2-trifluoroethyl)benzo[b]thiophen-7-yl)-1-methylpiperidin-4-amine (112.00 mg, 0.23 mmol), Pd(PPh₃)₂Cl₂ (37.00 mg, 0.052 mmol), DIEA (292.00 mg, 2.26 mmol), CuI (59.00 mg, 0.31 mmol), DMSO (5 mL), (4-methoxy-5-(prop-2-yn-1-ylamino)pyridin-2-yl)dimethylphosphine oxide (228.00 mg, 0.64 mmol). The reaction mixture was stirred at 50° C. for 3 h. The residue was applied onto a C₁₈ column eluted ACN/H₂O (0.01% TFA) (v:v=1:1). The residue was concentrated under vacuum. The resulting crude product was further purified by pre-HPLC with ACN/H₂O (0.1% ammonium hydroxide), Flow rate: 70 m L/min; Gradient: 30-66-66% B (2-30-32 min); 264 nm; RT: 26.97-29.11). This resulted in 73.00 mg (52.83% yield) of (5-((3-(7-(((3S,4R)-3-fluoro-1-methylpiperidin-4-yl)amino)-3-(2,2,2-trifluoroethyl)benzo[b]thiophen-2-yl) prop-2-yn-1-yl)amino)-4-methoxypyridin-2-yl)dimethylphosphine oxide (92) as white solid. LCMS: m/z=583 [M+1]⁺

¹H NMR (400 MHz, DMSO-d₆) δ 8.18 (s, 1H), 7.38 (d, J=5.6 Hz, 1H), 7.33-7.17 (m, 2H), 6.78 (d, J=7.8 Hz, 1H), 6.19-6.10 (m, 1H), 5.14 (d, J=8.5 Hz, 1H), 4.79 (d, J=49.6 Hz, 1H), 4.40 (d, J=6.3 Hz, 2H), 3.93 (s, 3H), 3.78-3.70 (m, 3H), 3.03 (t, J=10.8 Hz, 1H), 2.79-2.70 (m, 1H), 2.32-2.26 (m, 1H), 2.17 (d, J=10.4 Hz, 3H), 2.12-2.16 (m, 1H), 1.98-1.90 (m, 1H), 1.72 (d, J=11.2 Hz, 1H), 1.59 (d, J=13.4 Hz, 6H).

### Example 93

### (Z)-N-[2-[3-(4-dimethylphosphoryl-2-methoxy-anilino)prop-1-ynyl]-3-(2,2,2-trifluoroethyl)benzothiophen-7-yl]-1-(1,1-dioxothian-4-yl)-3-fluoro- piperidin-4-amine (93)

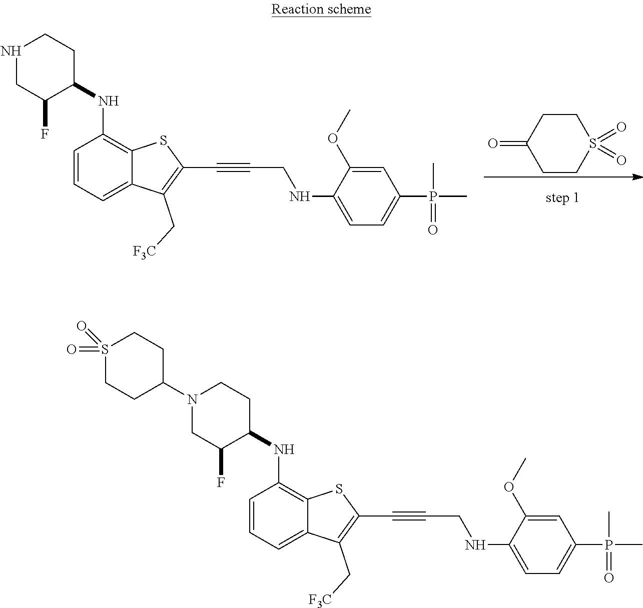 [SMILES: COc1cc(P(C)(C)=O)ccc1NCC#Cc1sc2c(N[C@@H]3CCNC[C@@H]3F)cccc2c1CC(F)(F)F ; O=C1CCS(=O)(=O)CC1 ; COc1cc(P(C)(C)=O)ccc1NCC#Cc1sc2c(N[C@@H]3CCN(C4CCS(=O)(=O)CC4)C[C@@H]3F)cccc2c1CC(F)(F)F]

### Experiment Details

### Step 1. Synthesis of (Z)—N-[2-[3-(4-dimethylphosphoryl-2-methoxy-anilino)prop-1-ynyl]-3-(2,2,2-trifluoroethyl)benzothiophen-7-yl]-1-(1,1-dioxothian-4-yl)-3-fluoro-piperidin-4-amine (93)

Into a 4-mL vial was placed (4-((3-(7-(((Z)-3-fluoropiperidin-4-yl)amino)-3-(2,2,2-trifluoroethyl)benzo[b]thiophen-2-yl)prop-2-yn-1-yl)amino)-3-methoxyphenyl)dimethylphosphine oxide (0.099 g, 174.43 μmol), tetrahydro-4H-thiopyran-4-one 1,1-dioxide (0.035 g, 236.20 μmol), sodium cyanoboronhydride (0.033 g, 769.66 μmol), EtOH (2 mL), AcOH (catalyst). The mixture was stirred at 60° C. overnight. The resulted reaction was purified by C₁₈ chromatography column eluted with ACN/water (0.1% ammonium bicarbonate) (v/v=1/1) to give the product. This resulted in 0.100 g (16.96% yield) of (Z)—N-[2-[3-(4-dimethylphosphoryl-2-methoxy-anilino)prop-1-ynyl]-3-(2,2,2-trifluoroethyl)benzothiophen-7-yl]-1-(1,1-dioxothian-4-yl)-3-fluoro-piperidin-4-amine (93) as white solid. LCMS: m/z=700 [M+1]⁺.

¹H NMR (400 MHz, DMSO-d6) δ 7.26 (q, J=6.6, 5.5 Hz, 1H), 7.20 (d, J=9.1 Hz, 2H), 7.14 (d, J=11.8 Hz, 1H), 6.84 (dd, J=8.2, 2.8 Hz, 1H), 6.78 (d, J=7.7 Hz, 1H), 6.05 (t, J=6.3 Hz, 1H), 5.13 (d, J=8.6 Hz, 1H), 4.87 (s, 1H), 4.75 (s, OH), 4.34 (d, J=6.1 Hz, 2H), 3.85 (s, 3H), 3.76 (dd, J=23.4, 12.1 Hz, 2H), 3.10 (d, J=16.1 Hz, 4H), 2.84 (d, J=11.1 Hz, 1H), 2.69 (s, 1H), 2.58 (d, J=12.9 Hz, 1H), 2.40-2.30 (m, 1H), 2.00-1.96 (m, 4H), 1.93-1.83 (m, 1H), 1.76 (dd, J=12.2, 5.0 Hz, 1H), 1.58 (d, J=13.1 Hz, 6H).

### Example 94

### N-(3,3-difluoro-1-methylpiperidin-4-yl)-4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-methoxybenzamide

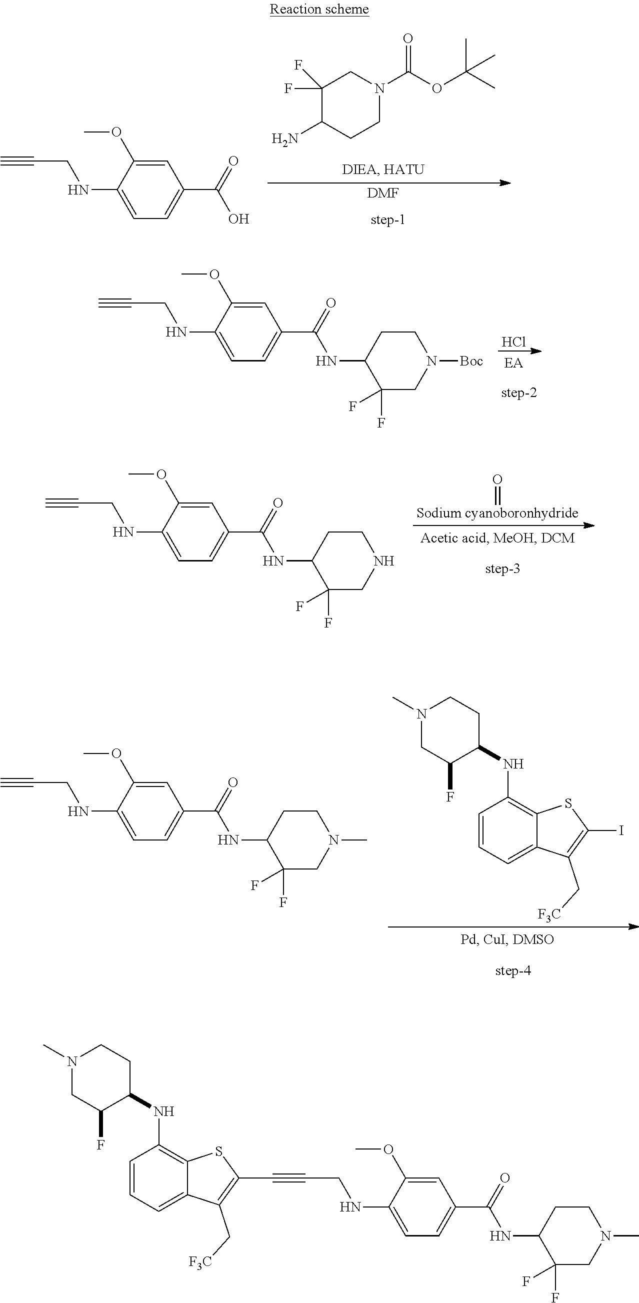

### Step 1. tert-butyl 3,3-difluoro-4-(3-methoxy-4-(prop-2-yn-1-ylamino)benzamido)piperidine-1-carboxylate

Into a 25-mL round-bottom flask purged and maintained with an inert atmosphere of nitrogen, was placed 3-methoxy-4-(prop-2-yn-1-ylamino)benzoic acid (0.349 g, 1.70 mmol), tert-butyl 4-amino-3,3-difluoropiperidine-1-carboxylate (0.408 g, 1.72 mmol), DIEA (0.683 g, 5.28 mmol), TATU (0.801 g, 2.10 mmol), DMF (5 mL). The reaction mixture was stirred at room temperature for 3 h. The reaction mixture was filtered through celite pad and celite pad was washed with ACN (3×20 mL). The filterate was then concentrated under reduced pressure to afford the crude product. The reaction mixture was purified by C18 column eluted with ACN/H₂O (v/v=2/1), filtered and concentrated under vacuum. This resulted in 0.629 g, (87.34% yield) of tert-butyl 3,3-difluoro-4-(3-methoxy-4-(prop-2-yn-1-ylamino)benzamido)piperidine-1-carboxylate as faint yellow solid. LCMS: m/z=424 [M+1]⁺.

### Step 2. N-(3,3-difluoropiperidin-4-yl)-3-methoxy-4-(prop-2-yn-1-ylamino)benzamide

Into a 25-mL round-bottom flask purged and maintained with an inert atmosphere of nitrogen, was placed tert-butyl 3,3-difluoro-4-(3-methoxy-4-(prop-2-yn-1-ylamino)benzamido)piperidine-1-carboxylate (0.62 g, 1.46 mmol), ethyl acetate (5 mL) and HCl in EA solution (5 mL, 4 M). The reaction mixture was stirred at room temperature for 1.5 h. The mixture was concentrated to afford the crude product. This resulted in 0.495 g (104.55% yield) of N-(3,3-difluoropiperidin-4-yl)-3-methoxy-4-(prop-2-yn-1-ylamino)benzamide as yellow oil. LCMS: m/z=324 [M+1]⁺.

### Step 3. N-(3,3-difluoro-1-methylpiperidin-4-yl)-3-methoxy-4-(prop-2-yn-1-ylamino)benzamide

Into a 25-mL three necked bottle purged and maintained with an inert atmosphere of nitrogen, was placed N-(3,3-difluoropiperidin-4-yl)-3-methoxy-4-(prop-2-yn-1-ylamino)benzamide (0.433 g, 1.33 mmol), formaldehyde (0.091 g, 3.03 mmol), Acetic acid (0.2 mL), MeOH (3 mL), DCM (3 mL) was added. The mixture was stirred at R.T. for 16 h. Sodium cyanoboronhydride, (0.664 g, 15.4866 mmol) was added. The reaction mixture was stirred at room temperature for 1 h. The reaction mixture was filtered through celite pad and celite pad was washed with ACN (3×20 mL). The filterate was then concentrated under reduced pressure to afford the crude product. The reaction mixture was purified by C18 column eluted with ACN/H₂O (v/v=2/3), filtered and concentrated under vacuum. This resulted in 0.225 g, (49.80% yield) of N-(3,3-difluoro-1-meth ylpiperidin-4-yl)-3-methoxy-4-(prop-2-yn-1-ylamino)benzamide as faint yellow oil. LCMS: m/z=338 [M+1]⁺

### Step 4 N-(3,3-difluoro-1-methylpiperidin-4-yl)-4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-methoxybenzamide (94)

Into a 10-mL round-bottom flask was placed N-(3,3-difluoro-1-methylpiperidin-4-yl)-3-methoxy-4-(prop-2-yn-1-ylamino)benzamide (0.081 g, 240.09 μmol), (Z)-3-fluoro-N-(2-iodo-3-(2,2,2-trifluoroethyl)benzo[b]thiophen-7-yl)-1-methylpiperidin-4-amine (0.215 g, 455.23 μmol), Pd(PPh₃)₂Cl₂ (0.031 g, 43.91 μmol), CuI (0.018 g, 94.51 μmol), DIEA (0.091 g, 704.10 μmol), methyl sulfoxide (2 mL). The reaction was stirred under nitrogen atmosphere at RT for 3 h. The reaction mixture was filtered through celite pad and celite pad was washed with ACN (3×20 mL). The filterate was then concentrated under reduced pressure to afford the crude product. The crude product was purified by preparative HPLC (Mobile Phase A: water (0.1% ammonium hydroxide), Mobile Phase B: ACN; Flow rate: 70 mL/min; Gradient: 30-60-85% B (2-30-60 min); 266 nm; RT: 39.091-40.870) to provide the desired product. This resulted in 0.061 g (37.26% yield) of N-(3,3-difluoro-1-meth ylpiperidin-4-yl)-4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-methoxybenzamide (94) as white solid. LCMS: m/z=682 [M+1]⁺.

¹H NMR (400 MHz, DMSO-d₆) δ 8.09 (d, J=8.9 Hz, 1H), 7.53 (d, J=8.2 Hz, 1H), 7.42 (s, 1H), 7.33-7.17 (m, 2H), 6.83-6.69 (m, 2H), 6.08 (t, J=6.4 Hz, 1H), 5.14 (d, J=8.5 Hz, 1H), 4.79 (d, J=49.4 Hz, 1H), 4.35 (d, J=6.4 Hz, 3H), 3.84 (d, J=21.6 Hz, 5H), 3.64 (d, J=29.4 Hz, 1H), 3.04 (d, J=12.7 Hz, 2H), 2.81 (d, J=10.7 Hz, 2H), 2.42-2.01 (m, 10H), 1.95-1.90 (m, 2H), 1.71 (d, J=11.3 Hz, 2H).

### Example 95

### 3-cyano-4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-N-methylbenzamide (racemic) (95)

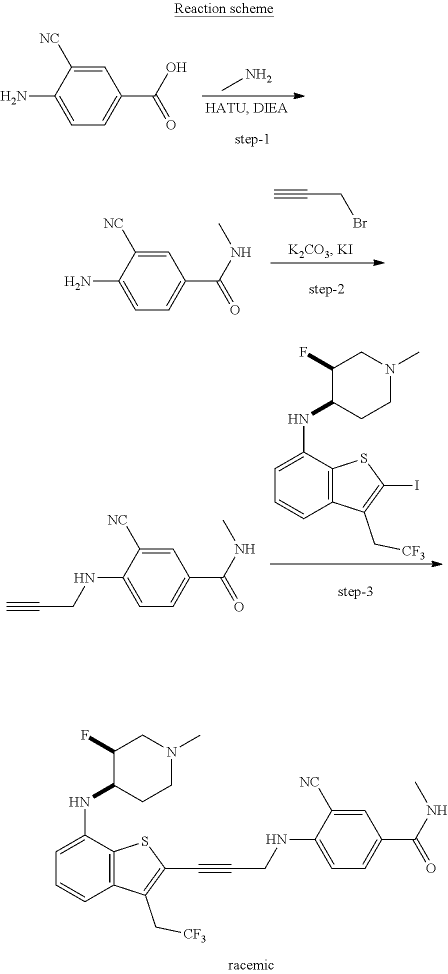

### Experimental Details

### Step 1. 4-amino-3-cyano-N-methylbenzamide

Into a 40-mL reaction vial purged and maintained with an inert atmosphere of nitrogen, was placed 4-amino-3-cyanobenzoic acid (353.00 mg, 2.17 mmol), Methylamine hydrochloride (495.00 mg, 7.33 mmol), HATU (1.21 g, 3.18 mmol), DIEA (1.41 g, 10.96 mmol), DMF (10 mL). The reaction mixture was stirred at room temperature for 2 h. The residue was applied onto a C₁₈ column eluted with ACN/H₂O (0.01% TFA) (v:v=1:1). This resulted in 362.00 mg (94.91% yield) of 4-amino-3-cyano-N-methylbenzamide as off-whit e solid. LCMS: m/z=176 [M+1]⁺

### Step 2. 3-cyano-N-methyl-4-(prop-2-yn-1-ylamino)benzamide

Into a 100-mL three necked bottle purged and maintained with an inert atmosphere of nitrogen, was placed 4-amino-3-cyano-N-methylbenzamide (345.00 mg, 1.97 mmol), K₂CO₃ (825.00 mg, 6.69 mmol), KI (3880.00 mg, 2.33 mmol), DMF (5 mL), 3-bromoprop-1-yne (1.36 g, 11.45 mmol). The reaction mixture was stirred at 80° C. for 4 h. The reaction mixture was filtered and the filter cake was washed with MeOH (20 mL). The residue was concentrated under vacuum. The residue was applied onto a C₁₈ column eluted with ACN/H₂O (0.01% TFA) (v:v=1:1). This resulted in 190.00 mg (45.24% yield) of 3-cyano-N-methyl-4-(prop-2-yn-1-ylamino)benzamide as yellow solid. LCMS: m/z=214 [M+1]⁺

### Step 3. 3-cyano-4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-N-methylbenzamide (racemic) (95)

Into a 40-mL three necked bottle purged and maintained with an inert atmosphere of nitrogen, was placed (Z)-3-fluoro-N-(2-iodo-3-(2,2,2-trifluoroethyl)benzo[b]thiophen-7-yl)-1-methylpiperidin-4-amine (50.00 mg, 0.11 mmol), 3-cyano-N-methyl-4-(prop-2-yn-1-ylamino)benzamide (130.00 mg, 0.61 mmol), Pd(PPh₃)₂Cl₂ (23.00 mg, 0.33 mmol), CuI (27.00 mg, 0.14 mmol), DMSO (3 mL), DIEA (62.00 mg, 0.48 mmol). The reaction mixture was stirred at 50° C. for 3 h. The resulting solution was added to H₂O (30 mL) and extracted with EA (3×50 mL). The organic layers combined and washed with brine (30 mL), dried over anhydrous Na₂SO₄, the residue was concentrated under vacuum. The resulting crude product was further purified by pre-HPLC with ACN/H₂O (0.1% ammonium hydroxide), Flow rate: 70 mL/min; Gradient: 35-65-85% B (2-30-60 min); 266 nm; RT: 32.153-34.242). This resulted in 13.00 mg (22.02% yield) of 3-cyano-4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-N-methylbenzamide (racemic) (95) as white solid. LCMS: m/z=558 [M+1]⁺

¹H NMR (400 MHz, DMSO-d₆) δ 8.21 (d, J=4.3 Hz, 1H), 7.96 (s, 1H), 7.90 (d, J=8.6 Hz, 1H), 7.28-7.09 (m, 3H), 6.96 (d, J=8.9 Hz, 1H), 6.72 (d, J=7.4 Hz, 1H), 5.35 (s, 1H), 4.89 (d, J=47.8 Hz, 1H), 4.36 (d, J=5.0 Hz, 2H), 3.90-3.60 (m, 3H), 3.27 (s, 2H), 3.10 (s, 2H), 2.75-2.57 (m, 4H), 2.54 (d, J=40.3 Hz, 2H), 2.00 (d, J=11.6 Hz, 1H), 1.81 (s, 1H).

### Example 96

### 4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-methoxy-N-(2-(2-methoxyethoxy)ethyl)benzamide (racemic) (96)

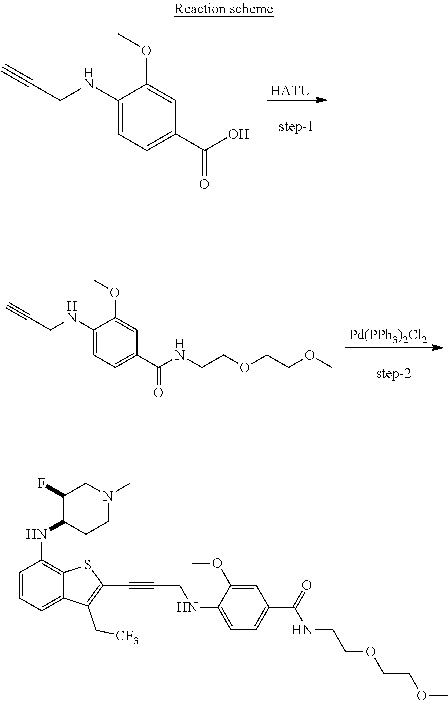 [SMILES: C#CCNc1ccc(C(=O)O)cc1OC ; "HATU" ; C#CCNc1ccc(C(=O)NCCOCCOC)cc1OC ; COCCOCCNC(=O)c1ccc(NCC#Cc2sc3c(N[C@@H]4CCN(C)C[C@@H]4F)cccc3c2CC(F)(F)F)c(OC)c1 ; "Pd(PPh3)2Cl2"]

### Experimental Details

### Step 1. 3-methoxy-N-(2-(2-methoxyethoxy)ethyl)-4-(prop-2-yn-1-ylamino)benzamide

Into a 100-mL three necked bottle purged and maintained with an inert atmosphere of nitrogen, was placed 3-methoxy-4-(prop-2-yn-1-ylamino)benzoic acid (0.61 g, 2.97 mmol), 2-(2-methoxyethoxy)ethan-1-amine (0.49 g, 4.11 mmol), HATU (1.41 g, 3.71 mmol), DIEA (0.90 g, 6.96 mmol), DMF (10 mL). The reaction mixture was stirred at 25° C. for 16 h. The reaction mixture was purified by C18 column eluted with ACN/H₂O (v/v=1/1). This resulted in 0.867 g (95.21% yield) of 3-methoxy-N-(2-(2-methoxyethoxy)ethyl)-4-(prop-2-yn-1-ylamino)benzamide as red solid. LCMS: m/z=307 [M+1]⁺.

### Step 2. 4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-methoxy-N-(2-(2-methoxyethoxy)ethyl)benzamide (racemic) (96)

Into a 50-mL three necked bottle and maintained with an inert atmosphere of nitrogen, was placed 3-meth oxy-N-(2-(2-methoxyethoxy)ethyl)-4-(prop-2-yn-1-ylamino)benzamide (0.126 g, 0.41 mmol), (Z)-3-fluoro-N-(2-iodo-3-(2,2,2-trifluoroethyl)benzo[b]thiophen-7-yl)-1-methylpiperidin-4-amine (racemic) (0.108 g, 0.23 mmol), CuI (0.018 g, 0.10 mmol), Pd(PPh₃)₂Cl₂ (0.064 g, 0.09 mmol), DIEA (0.117 g, 0.91 mmol) and DMSO (5 mL). The reaction mixture was stirred at room temperature for 1 h. LCMS showed that the reaction was completed. The mixture was purified by preparative HPLC (Mobile Phase A: water (10 mmoL/L ammonium hydroxide), Mobile Phase B: ACN; Flow rate: 40 m/min; Gradient: 35-70-70% B (2-30-60 min); 266 nm; RT: 30.375-34.583 min). This resulted in 0.021 g (14.11% yield) of 4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-methoxy-N-(2-(2-methoxyethoxy)ethyl)benzamide (racemic) (96) as off white solid. LCMS: m/z=651 [M+1]⁺.

¹H NMR (400 MHz, DMSO) δ 8.20 (t, J=5.4 Hz, 1H), 7.45 (d, J=8.3 Hz, 1H), 7.37 (s, 1H), 7.27 (t, J=7.8 Hz, 1H), 7.20 (d, J=7.9 Hz, 1H), 6.84-6.72 (m, 2H), 6.04 (t, J=6.2 Hz, 1H), 5.15 (d, J=8.5 Hz, 1H), 4.79 (d, J=49.6 Hz, 1H), 4.34 (d, J=6.2 Hz, 2H), 3.84 (d, J=7.3 Hz, 3H), 3.83-3.75 (m, 2H), 3.73-3.58 (m, 1H), 3.56-3.48 (m, 4H), 3.47-3.41 (m, 2H), 3.41-3.35 (m, 2H), 3.23 (s, 3H), 3.02 (t, J=11.2 Hz, 1H), 2.79 (d, J=10.6 Hz, 1H), 2.26 (d, J=12.7 Hz, 1H), 2.17 (s, 3H), 2.07 (t, J=11.3 Hz, 1H), 2.01-1.88 (m, 1H), 1.71 (d, J=10.9 Hz, 1H).

### Example 97

### 1-[(Z)-4-[[2-[3-(4-dimethylphosphoryl-2-methoxy-anilino)prop-1-ynyl]-3-(2,2,2-trifluoroethyl)benzothiophen-7-yl]amino]-3-fluoro-1-piperidyl]-3-methoxy-propan-2-ol (97)

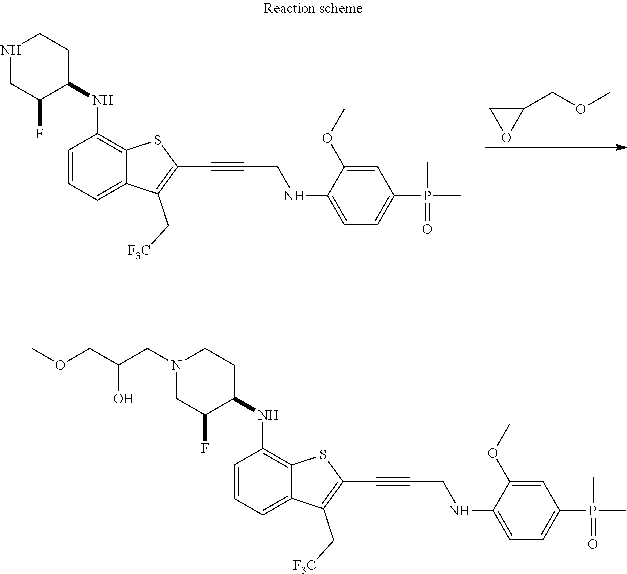

### Experiment Details

### Step 1. Synthesis of 1-[(Z)-4-[[2-[3-(4-dimethylphosphoryl-2-methoxy-anilino)prop-1-ynyl]-3-(2,2,2-trifluoroethyl)benzothiophen-7-yl]amino]-3-fluoro-1-piperidyl]-3-methoxy-propan-2-ol (97)

Into a 4-mL vial was placed (4-((3-(7-(((Z)-3-fluoropiperidin-4-yl)amino)-3-(2,2,2-trifluoroethyl)benzo[b]thiophen-2-yl)prop-2-yn-1-yl)amino)-3-methoxyphenyl)dimethylphosphine oxide (0.078 g, 137.43 μmol), MeOH (1 mL), 2-(methoxymethyl)oxirane (0.054 g, 612.90 μmol). The mixture was stirred at 50° C. overnight. The resulted reaction was purified by C₁₈ chromatography column eluted with ACN/water (0.1% ammonium bicarbonate) (v/v=1/1) to give the product. This resulted in 0.063 g (69.92% yield) of 1-[(Z)-4-[[2-[3-(4-dimethylphosphoryl-2-methoxy-anilino)prop-1-ynyl]-3-(2,2,2-trifluoroethyl)benzothiophen-7-yl]amino]-3-fluoro-1-piperidyl]-3-methoxy-propan-2-ol (97) as white solid. LCMS: m/z=656 [M+1]⁺.

¹H NMR (400 MHz, DMSO-d6) δ 7.27 (t, J=7.8 Hz, 1H), 7.21 (dd, J=11.2, 8.0 Hz, 2H), 7.14 (dd, J=11.8, 1.6 Hz, 1H), 6.84 (dd, J=8.0, 2.9 Hz, 1H), 6.78 (d, J=7.7 Hz, 1H), 6.05 (t, J=6.4 Hz, 1H), 5.12 (d, J=8.5 Hz, 1H), 4.84 (s, OH), 4.72 (s, 1H), 4.53 (t, J=6.2 Hz, 1H), 4.34 (d, J=6.4 Hz, 2H), 3.85 (s, 3H), 3.79 (d, J=11.0 Hz, 1H), 3.72 (s, 2H), 3.32 (s, 4H), 3.35-3.27 (m, 1H), 3.25-3.19 (m, 1H), 3.13 (dd, J=21.9, 11.0 Hz, 1H), 2.97-2.85 (m, 1H), 2.46-2.19 (m, 4H), 1.99-1.86 (m, 1H), 1.71 (d, J=12.9 Hz, 1H), 1.58 (d, J=13.1 Hz, 6H).

### Example 98

(Z)—N-[2-[3-(4-dimethylphosphoryl-2-methoxy-anilino)prop-1-ynyl]-3-(2,2,2-trifluoroethyl)benzothiophen-7-yl]-3-fluoro-1-(trifluoromethyl)piperidin-4-amine (98)

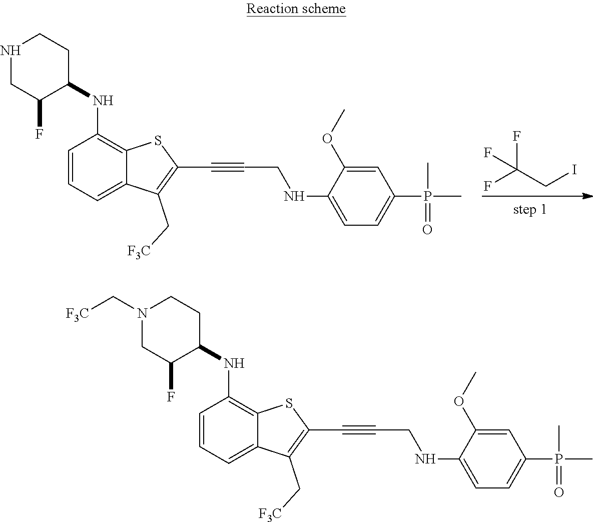

### Experiment Details

### Step 1. Synthesis of (Z)—N-[2-[3-(4-dimethylphosphoryl-2-methoxy-anilino)prop-1-ynyl]-3-(2,2,2-trifluoroethyl)benzothiophen-7-yl]-3-fluoro-1-(trifluoromethyl)piperidin-4-amine (98)

Into a 4-mL vial was placed (4-((3-(7-(((Z)-3-fluoropiperidin-4-yl)amino)-3-(2,2,2-trifluoroethyl)benzo[b]thiophen-2-yl)prop-2-yn-1-yl)amino)-3-methoxyphenyl)dimethylphosphine oxide (0.102 g, 179.71 μmol), N,N-Dimeth ylformamide (2 mL), N,N-Diisopropylethylamine (0.170 g, 1.32 mmol), amine 2,2,2-Trifluoroethyltrifluoromethanesulfonate (0.087 g, 374.84 μmol). The mixture was stirred at 50° C. overnight. The resulted reaction was purified by C₁₈ chromatography column eluted with ACN/water (0.1% ammonium bicarbonate) (v/v=1/1) to give the product. This resulted in 0.052 g (44.54% yield) of (Z)—N-[2-[3-(4-dimethylphosphoryl-2-methoxy-anilino)prop-1-ynyl]-3-(2,2,2-trifluoroethyl)benzothiophen-7-yl]-3-fluoro-1-(2,2,2-trifluoroethyl)piperidin-4-amine (98) as white solid. LCMS: m/z=650 [M+1]⁺.

¹H NMR (400 MHz, DMSO-d6) δ 7.26 (q, J=6.2, 4.5 Hz, 1H), 7.20 (d, J=9.0 Hz, 2H), 7.14 (d, J=11.9 Hz, 1H), 6.84 (dd, J=8.6, 2.8 Hz, 1H), 6.79 (d, J=7.6 Hz, 1H), 6.05 (t, J=6.4 Hz, 1H), 5.16 (d, J=8.6 Hz, 1H), 4.86 (s, 1H), 4.73 (s, 1H), 4.34 (d, J=6.1 Hz, 2H), 3.85 (s, 3H), 3.79 (q, J=10.8 Hz, 2H), 3.29 (s, 2H), 3.25 (d, J=10.5 Hz, 1H), 3.20 (t, J=11.2 Hz, 1H), 2.96 (d, J=11.8 Hz, 1H), 2.78 (dd, J=38.6, 13.3 Hz, 1H), 2.63 (t, J=12.5 Hz, 1H), 2.03-1.90 (m, 1H), 1.70 (d, J=12.5 Hz, 1H), 1.58 (d, J=13.1 Hz, 6H).

### Example 99

### dicyclopropyl(4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)phosphine oxide (racemic) (99)

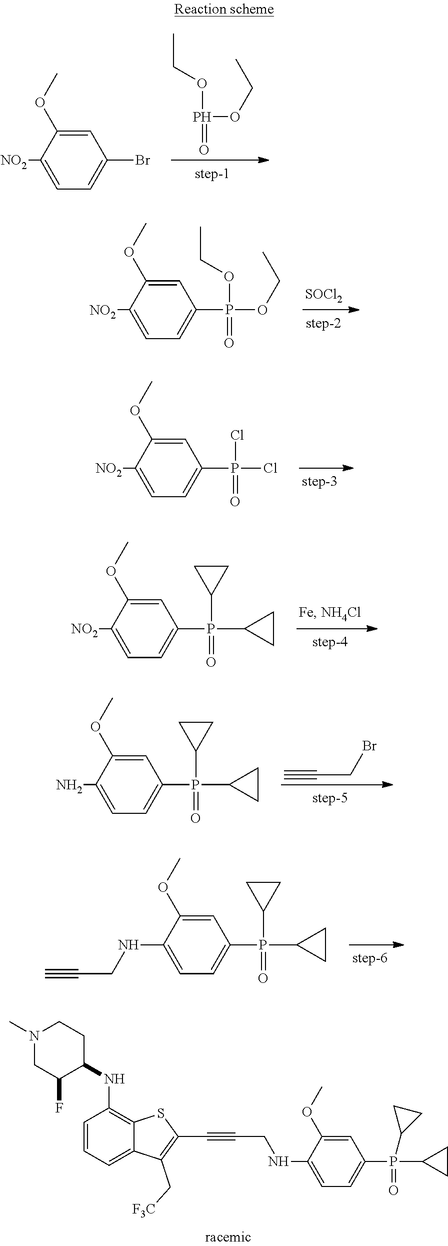

### Step 1. diethyl (3-methoxy-4-nitrophenyl)phosphonate

Into a 250-mL three necked bottle purged and maintained with an inert atmosphere of nitrogen, was placed 4-bromo-2-methoxy-1-nitrobenzene (15.12 g, 65.16 mmol), Pd(OAc)₂ (1.59 g, 7.08 mmol), Xantphos (7.63 g, 13.18 mmol), DIEA (25.42 g, 196.68 mmol), DMF (60 mL) and stirred at 110° C. for 5 min, then added diethyl phosphonate (12.90 g, 93.41 mmol) in DMF (5 mL). The reaction mixture was stirred at 110° C. for 2 h. The reaction mixture was filtered. The residue was concentrated under vacuum. The residue was applied onto a C₁₈ column eluted with ACN/H₂O (0.01% TFA) (v:v=1:1), Flow rate: 100 mL/min. This resulted in 16.48 g (87.44% yield) of diethyl (3-methoxy-4-nitrophenyl)phosphonate as brown oil. LCMS: m/z=290 [M+1]⁺

### Step 2. (3-methoxy-4-nitrophenyl)phosphonic dichloride

Into a 100-mL three necked bottle purged and maintained with an inert atmosphere of nitrogen, was placed diethyl (3-methoxy-4-nitrophenyl)phosphonate (1.09 g, 3.76 mmol), DMF (0.33 g, 4.51 mmol), SOCl₂ (12.86 g, 108.09 mmol). The reaction mixture was stirred at 80° C. for 12 h. The resulting solution was concentrated under vacuum. This resulted in 1.16 g (113.99% yield) of (3-methoxy-4-nitrophenyl)phosphonic dichloride as yellow oil. LCMS: m/z=270 [M+1]⁺

### Step 3. dicyclopropyl(3-methoxy-4-nitrophenyl)phosphine oxide

Into a 250-mL three necked bottle purged and maintained with an inert atmosphere of nitrogen, was placed (3-methoxy-4-nitrophenyl)phosphonic dichloride (7.02 g, 25.99 mmol), THF (50 mL). The reaction mixture was cooled to −70° C. Cyclopropylmagnesium bromide (1 M in THF, 50 mL, 50 mmol) was added and stirred at −50° C.˜−20° C. for 30 minutes. The resulting solution was quenched with H₂O (100 mL) and extracted with EA (2×100 mL). The organic layers combined and washed with brine (100 mL), dried over anhydrous Na₂SO₄, the residue was concentrated under vacuum. The residue was applied onto a C₁₈ column eluted with ACN/H₂O (0.01% TFA) (v:v=1:1), Flow rate: 100 m/min. This resulted in 1.50 g (20.51% yield) of dicyclopropyl(3-methoxy-4-nitrophenyl)phosphine oxide as yellow oil. LCMS: m/z=281 [M+1]⁺

### Step 4. (4-amino-3-methoxyphenyl)dicyclopropylphosphine oxide

Into a 100-mL three necked bottle and maintained with an inert atmosphere of nitrogen, was placed dicyclopropyl(3-methoxy-4-nitrophenyl)phosphine oxide (1.83 g, 6.50 mmol), Fe(2.20 g, 39.39 mmol), NH₄Cl (2.15 g, 40.19 mmol), ETOH (30 mL), H₂O (5 mL). The reaction mixture was stirred at 80° C. for 3 h. The reaction mixture was filtered and the filter cake was washed with EtOH (20 mL). The residue was concentrated under vacuum. The residue was applied onto a C₁₈ column eluted with ACN/H₂O (0.01% TFA) (v:v=1:1), Flow rate: 100 m/min. This resulted in 1.40 g (85.63% yield) of (4-amino-3-methoxyphenyl)dicyclopropylphosphine oxide as yellow solid. LCMS: m/z=252 [M+1]⁺

### Step 5. dicyclopropyl(3-methoxy-4-(prop-2-yn-1-ylamino)phenyl)phosphine oxide

Into a 50-mL three necked bottle purged and maintained with an inert atmosphere of nitrogen, was placed (4-amino-3-methoxyphenyl)dicyclopropylphosphine oxide (1.32 g, 5.25 mmol), 3-bromoprop-1-yne (2.32 g, 19.50 mmol), DIEA (3.15 g, 24.37 mmol), MeCN (20 mL). The reaction mixture was stirred at 80° C. for 5 h. The residue was concentrated under vacuum. The residue was applied onto a C₁₈ column eluted with ACN/H₂O (0.01% TFA) (v:v=1:1), Flow rate: 100 mL/min. This resulted in 1.01 g (66.32% yield) of dicyclo propyl(3-methoxy-4-(prop-2-yn-1-ylamino)phenyl)phosphine oxide as brown oil. LCMS: m/z=290 [M+1]⁺

### Step 6. dicyclopropyl(4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)phosphine oxide (racemic) (99)

Into a 50-mL three necked bottle purged and maintained with an inert atmosphere of nitrogen, was placed (Z)-3-fluoro-N-(2-iodo-3-(2,2,2-trifluoroethyl)benzo[b]thiophen-7-yl)-1-methylpiperidin-4-amine (165.00 mg, 0.1 mmol), dicyclopropyl(3-methoxy-4-(prop-2-yn-1-ylamino)phenyl)phosphine oxide (367.00 mg, 1.26 mmol), Pd(PPh₃)₂Cl₂ (60.00 mg, 0.085 mmol), CuI (73.00 mg, 0.38 mmol), DIEA (314.00 mg, 2.43 mmol), DMSO (8 mL). The reaction mixture was stirred at 50° C. for 3 h. The residue was applied onto a C₁₈ column eluted with ACN/H₂O (0.01% TFA) (v:v=1:1), Flow rate: 80 mL/min. the residue was concentrated under vacuum. The resulting crude product was further purified by pre-HPLC with ACN/H₂O (0.1% ammonium hydroxide), Flow rate: 70 mL/min; Gradient: 35-70-95% B (2-30-60 min); 266 nm; RT: 33.703-37.730). This resulted in 107.00 mg (48.33% yield) of dicyclopropyl(4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)phosphine oxide (racemic) (99) as off white solid. LCMS: m/z=634 [M+1]⁺

¹H NMR (400 MHz, DMSO-d₆) δ 7.30-7.20 (m, 3H), 7.15 (d, J=11.3 Hz, 1H), 6.89-3.83 (m, 1H), 6.77 (t, J=7.7 Hz, 1H), 6.08 (t, J=6.2 Hz, 1H), 5.16 (d, J=8.5 Hz, 1H), 4.80 (d, J=49.5 Hz, 1H), 4.34 (d, J=6.2 Hz, 2H), 4.00-3.74 (m, 6H), 3.74-3.55 (m, 1H), 3.04 (t, J=10.7 Hz, 1H), 2.80 (d, J=10.7 Hz, 1H), 2.29-2.05 (m, 5H), 2.01-1.87 (m, 1H), 1.80-1.65 (m, 1H), 1.28-1.10 (m, 2H), 0.79-0.59 (m, 7H).

### Example 100

### (4-((3-(7-(((Z)-1-ethyl-3-fluoropiperidin-4-yl)amino)-3-(2,2,2-trifluoroethyl)benzo[b]thiophen-2-yl)prop-2-yn-1-yl)amino)-3-methoxyphenyl)dimethylphosphine oxide(racemic) (100)

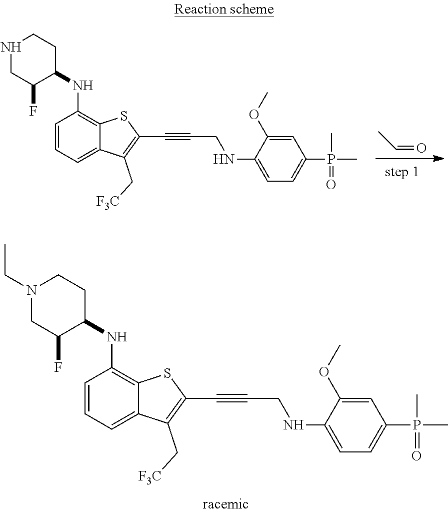

### Experimental Details

### Step 1. (4-((3-(7-(((Z)-1-ethyl-3-fluoropiperidin-4-yl)amino)-3-(2,2,2-trifluoroethyl)benzo[b]thiophen-2-yl)prop-2-yn-1-yl)amino)-3-methoxyphenyl)dimethylphosphine oxide(racemic) (100)

Into a 4-mL sealed tube purged and maintained with an inert atmosphere of nitrogen, was placed acetaldehyde (0.16 g, 3.63 mmol), methanol (1 mL), (4-((3-(7-(((Z)-3-fluoropiperidin-4-yl)amino)-3-(2,2,2-trifluoroethyl)benzo[b]thiophen-2-yl)prop-2-yn-1-yl)amino)-3-methoxyphenyl)dimethylphosphine oxide (0.061 g, 107.47 μmol), sodium cyanoboronhydride (0.025 g, 583.08 μmol). The reaction mixture was stirred at 50° C. The mixture was purified by preparative HPLC (Mobile Phase A: water (10 mmoL/L ammonium hydroxide), Mobil e Phase B: ACN; Flow rate: 70 mL/min; Gradient: 35-65-85% B (2-30-60 min); 264 nm; RT: 33.120-34.960 min). This resulted in 0.184 g (83.89% yield) of (4-((3-(7-(((Z)-1-ethyl-3-fluoropiperidin-4-yl)amino)-3-(2,2,2-trifluoroethyl)benzo[b]thiophen-2-yl)prop-2-yn-1-yl)amino)-3-methoxyphenyl)dimethylphosphine oxide(racemic) (100) as yellow solid. LCMS: m/z=596 [M+1]⁺.

¹H NMR (400 MHz, DMSO) δ 7.28 (d, J=8.1 Hz, 1H), 7.25 (s, 1H), 7.21 (s, 1H), 7.19 (s, 1H), 7.14 (d, J=12.1 Hz, 1H), 6.84 (d, J=5.8 Hz, 1H), 6.78 (d, J=7.3 Hz, 1H), 6.05 (s, 1H), 5.12 (d, J=8.4 Hz, 1H), 4.87 (s, 1H), 4.74 (s, 1H), 4.34 (d, J=6.3 Hz, 2H), 3.85 (s, 3H), 3.79 (d, J=10.3 Hz, 2H), 2.41-2.32 (m, 3H), 2.10-2.03 (m, 1H), 1.94-1.87 (m, 1H), 1.75 (s, 2H), 1.58 (d, J=13.2 Hz, 6H), 0.99 (t, J=7.1 Hz, 3H).

### Example 101

### (4-((3-(7-(((Z)-3-fluoro-1-isobutylpiperidin-4-yl)amino)-3-(2,2,2-trifluoroethyl)benzo[b]thiophen-2-yl)prop-2-yn-1-yl)amino)-3-methoxyphenyl)dimethylphosphine oxide(racemic) (101)

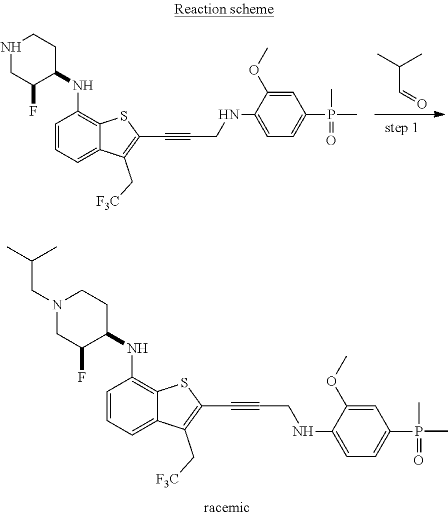

### Experimental Details

### Step 1. (4-((3-(7-(((Z)-3-fluoro-1-isobutylpiperidin-4-yl)amino)-3-(2,2,2-trifluoroethyl)benzo[b]thiophen-2-yl) prop-2-yn-1-yl)amino)-3-methoxyphenyl)dimethylphosphine oxide(racemic) (101)

Into a 8-mL sealed tube purged and maintained with an inert atmosphere of nitrogen, was placed isobutyraldehyde (0.014 g, 194.16 μmol), sodium cyanoboronhydride (0.048 g, 1.12 mmol), methanol (2 mL), acetic acid (0.1 mL), (4-((3-(7-(((Z)-3-fluoropiperidin-4-yl)amino)-3-(2,2,2-trifluoroethyl)benzo[b]thiophen-2-yl)prop-2-yn-1-yl)amino)-3-methoxyphenyl)dimethylphosphine oxide (0.103 g, 181.47 μmol). The reaction mixture w as stirred at 50° C. for 22 h. The reaction was quenched by the addition of water (5 mL) and extracted with EA (2×10 mL). The organic layer was combined, washed with brine (10 mL), dried over anhydrous Na₂SO₄ and concentrated under vacuum. The mixture was purified by preparative HPLC (Mobile Phase A: water (10 mmoL/L ammonium hydroxide), Mobile Phase B: ACN; Flow rate: 70 mL/min; Gradient: 40-65-90% B (2-30-60 min); 266 nm; RT: 50.313-54.108 min). This resulted in 0.028 g (24.74% yield) of (4-((3-(7-(((Z)-3-fluoro-1-isobutylpiperidin-4-yl)amino)-3-(2,2,2-trifluoroethyl)benzo[b]thiophen-2-yl)prop-2-yn-1-yl)amino)-3-methoxyphenyl)dimethylphosphine oxide(racemic) (101) as white solid. LCMS: m/z=624 [M+1]⁺.

¹H NMR (400 MHz, DMSO) δ 7.27 (s, 1H), 7.25 (s, 1H), 7.21 (s, 1H), 7.19 (s, 1H), 7.17-7.11 (m, 1H), 6.88-6.82 (m, 1H), 6.78 (d, J=7.6 Hz, 1H), 6.05 (s, 1H), 5.12 (d, J=8.6 Hz, 1H), 4.85 (s, 1H), 4.73 (s, 1H), 4.34 (d, J=6.5 Hz, 2H), 3.85 (s, 4H), 3.80 (s, 1H), 3.78 (s, 1H), 2.06 (d, J=8.1 Hz, 3H), 1.96 (s, 1H), 1.75 (s, 3H), 1.59 (s, 3H), 1.56 (s, 3H), 0.85 (d, J=6.4 Hz, 6H).

### Example 102

### (4-((3-(7-(((Z)-1-(cyclopropylmethyl)-3-fluoropiperidin-4-yl)amino)-3-(2,2,2-trifluoroethyl)benzo[b]thiophen-2-yl)prop-2-yn-1-yl)amino)-3-methoxyphenyl)dimethylphosphine oxide (102)

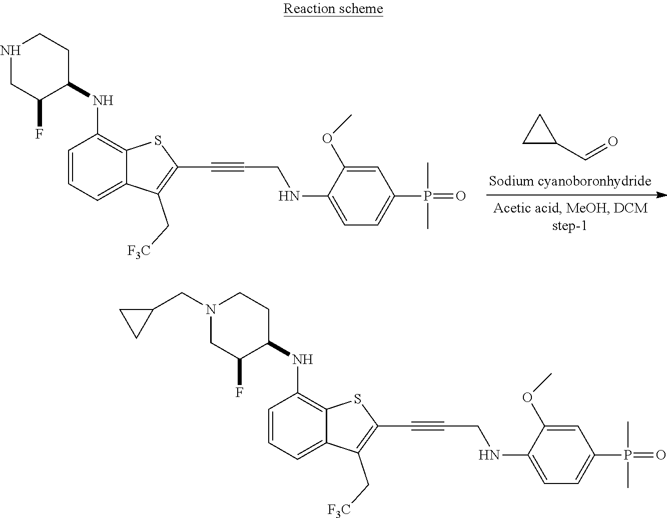

### Experimental Details

### Step 1. (4-((3-(7-(((Z)-1-(cyclopropylmethyl)-3-fluoropiperidin-4-yl)amino)-3-(2,2,2-trifluoroethyl)benzo[b]thiophen-2-yl)prop-2-yn-1-yl)amino)-3-methoxyphenyl)dimethylphosphine oxide (102)

Into a 8-mL sealed tube and maintained with an inert atmosphere of nitrogen, was placed (4-((3-(7-(((Z)-3-fluoropiperidin-4-yl)amino)-3-(2,2,2-trifluoroethyl)benzo[b]thiophen-2-yl)prop-2-yn-1-yl)amino)-3-methoxyphenyl) dimethylphosphine oxide (0.051 g, 89.85 μmol), cyclopropanecarbaldehyde (0.034 g, 485.09 μmol), Acetic acid (0.2 mL), methanol (0.5 mL), DCM (0.5 mL). Then Sodium cyanoboronhydride (0.062 g, 1.44 mmol) was added. The mixture was stirred at R.T. for 16 h. The reaction mixture was filtered through celite pad and the celite pad was washed with ACN (3×20 mL). The filtrate was then concentrated under reduced pressure to afford the crude product. The reaction mixture was purified by C18 column eluted with ACN/H₂O (v/v=1/2), filtered and concentrated under vacuum. This resulted in 0.029 g, (51.91% yield) of (4-((3-(7-(((Z)-1-(cyclopropylmethyl)-3-fluoropiperidin-4-yl)amino)-3-(2,2,2-trifluoroethyl)benzo[b]thiophen-2-yl)prop-2-yn-1-yl) amino)-3-methoxyphenyl)dimethylphosphine oxide (102) as yellow solid. LCMS: m/z=622 [M+1]⁺.

¹H NMR (400 MHz, DMSO-d₆) δ 7.33-7.10 (m, 4H), 6.90-6.73 (m, 2H), 6.05 (t, J=6.3 Hz, 1H), 5.12 (d, J=8.5 Hz, 1H), 4.81 (d, J=49.4 Hz, 1H), 4.34 (d, J=6.4 Hz, 2H), 3.81 (d, J=29.1 Hz, 6H), 3.22 (t, J=11.3 Hz, 1H), 2.99 (d, J=11.3 Hz, 1H), 2.41-2.10 (m, 4H), 2.01-1.87 (m, 1H), 1.80-1.70 (m, 1H), 1.58 (d, J=13.1 Hz, 6H), 0.87-0.77 (m, 1H), 0.46 (dt, J=8.4, 2.8 Hz, 2H), 0.06 (t, J=4.7 Hz, 2H).

### Example 103

### (4-((3-(7-(((Z)-1-cyclobutyl-3-fluoropiperidin-4-yl)amino)-3-(2,2,2-trifluoroethyl)benzo[b]thiophen-2-yl)prop-2-yn-1-yl)amino)-3-methoxyphenyl)dimethylphosphine oxide (racemic) (103)

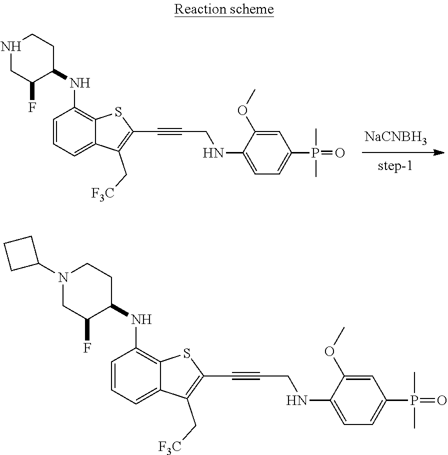

### Experimental Details

### Step 1. (4-((3-(7-(((Z)-1-cyclobutyl-3-fluoropiperidin-4-yl)amino)-3-(2,2,2-trifluoroethyl)benzo[b]thiophen-2-yl)prop-2-yn-1-yl)amino)-3-methoxyphenyl)dimethylphosphine oxide (racemic) (103)

Into a 100-mL three necked bottle purged and maintained with an inert atmosphere of nitrogen, was placed (4-((3-(7-(((Z)-3-fluoropiperidin-4-yl)amino)-3-(2,2,2-trifluoroethyl)benzo[b]thiophen-2-yl)prop-2-yn-1-yl)amino)-3-methoxyphenyl)dimethylphosphine oxide (racemic) (0.097 g, 0.17 mmol), cyclobutanone (0.110 g, 1.57 m mol), NaCNBH₃ (0.186 g, 4.34 mmol), MeOH (5 mL). The reaction mixture was stirred at 50° C. for 6 h. The resulting solution was added to water (300 mL). The resulting solution was extracted with EA (2×1 50 mL). The organic layers combined and washed with brine (20 mL), dried over anhydrous Na₂SO₄, the residue was concentrated under vacuum. The mixture was purified by preparative HPLC (Mobile Phase A: water (10 mmoL/L ammonium hydroxide), Mobile Phase B: ACN; Flow rate: 40 mL/min; Gradient: 40-65-90% B (2-30-60 min); 266 nm; RT: 36.378-39.062 min). This resulted in 0.035 g (32.94% yield) of (4-((3-(7-(((Z)-1-cyclobutyl-3-fluoropiperidin-4-yl)amino)-3-(2,2,2-trifluoroethyl)benzo[b]thiophen-2-yl)prop-2-yn-1-yl)amino)-3-methoxyphenyl)dimethylphosphine oxide (racemic) (103) as off white solid. LCMS: m/z=622 [M+1]⁺.

¹H NMR (400 MHz, DMSO) δ 7.31-7.10 (m, 4H), 6.91-6.71 (m, 2H), 6.05 (t, J=6.3 Hz, 1H), 5.11 (d, J=8.5 Hz, 1H), 4.80 (d, J=49.6 Hz, 1H), 4.34 (d, J=6.3 Hz, 2H), 3.85 (s, 3H), 3.83-3.73 (m, 2H), 3.72-3.58 (m, 1H), 3.01 (t, J=10.7 Hz, 1H), 2.86-2.69 (m, 2H), 2.12 (d, J=12.8 Hz, 1H), 2.06-1.86 (m, 4H), 1.85-1.61 (m, 5H), 1.60 (s, 3H), 1.56 (s, 3H).

### Example 104

### (4-((3-(7-(((Z)-1-cyclopentyl-3-fluoropiperidin-4-yl)amino)-3-(2,2,2-trifluoroethyl)benzo[b]thiophen-2-yl)prop-2-yn-1-yl)amino)-3-methoxyphenyl)dimethylphosphine oxide (104)

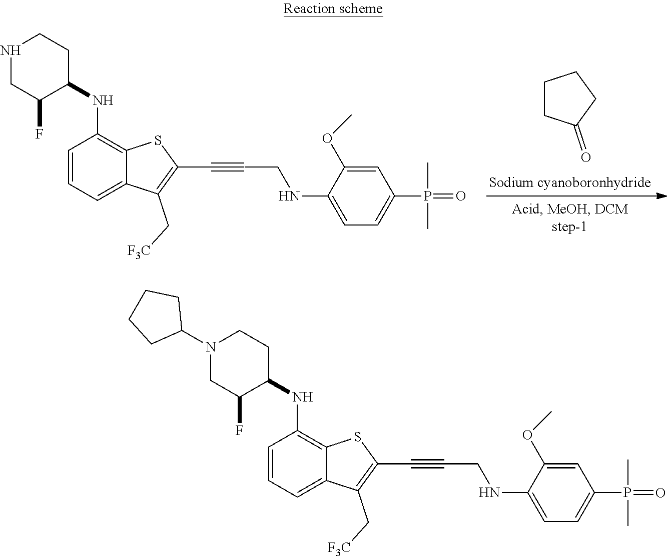

### Step 1. (4-((3-(7-(((Z)-1-cyclopentyl-3-fluoropiperidin-4-yl)amino)-3-(2,2,2-trifluoroethyl)benzo[b]thiophen-2-yl)prop-2-yn-1-yl)amino)-3-methoxyphenyl)dimethylphosphine oxide (104)

Into a 8-mL sealed tube and maintained with an inert atmosphere of nitrogen, was placed (4-((3-(7-(((Z)-3-fluoropiperidin-4-yl)amino)-3-(2,2,2-trifluoroethyl)benzo[b]thiophen-2-yl)prop-2-yn-1-yl)amino)-3-methoxyphenyl) dimethylphosphine oxide (0.051 g, 89.85 μmol), cyclopentanone (0.034 g, 404.20 μmol), Acetic acid (0.2 mL), methanol (0.5 mL), DCM (0.5 mL). Then Sodium cyanoboronhydride (0.064 g, 1.49 mmol) was added. The mixture was stirred at rt for 16 h. The reaction mixture was filtered through celite pad and celite pad was washed with ACN (3×20 mL). The filterate was then concentrated under reduced pressure to afford the crude product. The reaction mixture was purified by C18 column eluted with ACN/H₂O (v/v=1/2), filtered and concentrated under vacuum. This resulted in 0.018 g, (31.51% yield) of (4-((3-(7-(((Z)-1-cyclopentyl-3-fluoropiperidin-4-yl)amino)-3-(2,2,2-trifluoroethyl)benzo[b]thiophen-2-yl)prop-2-yn-1-yl)amino)-3-methoxyphenyl)dimethylphosphine oxide (104) yellow solid. LCMS: m/z=636 [M+1]⁺.

¹H NMR (400 MHz, DMSO-d₆) δ 7.20-7.17 (m, 4H), 6.91-6.73 (m, 2H), 6.08-6.05 (m, 1H), 5.08 (t, J=8.7 Hz, 1H), 4.90-4.66 (m, 1H), 4.33 (t, J=7.5 Hz, 2H), 3.83-3.79 (m, 6H), 3.15 (d, J=10.1 Hz, 1H), 2.91 (d, J=9.5 Hz, 1H), 2.33-2.04 (m, 2H), 1.95-1.84 (m, 1H), 1.74 (s, 3H), 1.63-1.41 (m, 10H), 1.40-1.20 (m, 3H).

### Example 105

### (4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-(trifluoromethyl)phenyl)dimethylphosphine oxide (105)

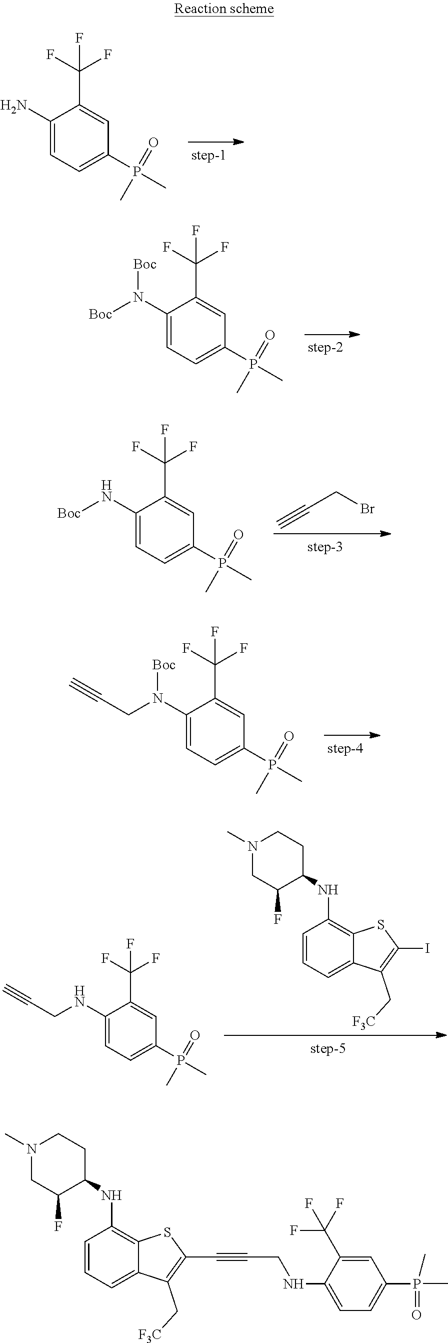

### Experiment Details

### Step 1. tert-butyl N-tert-butoxycarbonyl-N-[4-dimethylphosphoryl-2-(trifluoromethyl)phenyl]carbamate

Into a 20 mL vial was placed (4-amino-3-(trifluoromethyl)phenyl)dimethylphosphine oxide (0.21 g, 0.87 m mol), Di-tert-butyl dicarbonate (0.23 g, 1.03 mmol), N-(4-pyridyl)dimethylamine (0.27 g, 2.18 mmol), 1,4-Dioxane (5 mL). The reaction was stirred at 110° C. overnight. The mixture was concentrated under vacuum. This resulted in 0.316 g (83.17% yield) of tert-butyl N-tert-butoxycarbonyl-N-[4-dimethylphosphoryl-2-(trifluoromethyl)phenyl]carbamate as off-white solid. LCMS: m/z=438 [M+1]⁺.

### Step 2. tert-butyl (4-(dimethylphosphoryl)-2-(trifluoromethyl)phenyl)carbamate

Into a 40 mL vial was placed tert-butyl N-tert-butoxycarbonyl-N-[4-dimethylphosphoryl-2-(trifluoromethyl)phenyl]carbamate (0.26 g, 0.60 mmol), K₂CO₃ (0.70 g, 5.08 mmol), MeOH (5 mL). The reaction was stirred at 70° C. for 1 h. The reaction was quenched by water (3 mL). The mixture was purified by flash-C18 eluted with ACN/water (V/V=3/7) to provide the desired product. This resulted in 0.169 g (83.02% yield) of tert-butyl (4-(dimethylphosphoryl)-2-(trifluoromethyl)phenyl)carbamate as brown solid. LCMS: m/z=338 [M+1]⁺.

### Step 3. tert-butyl (4-(dimethylphosphoryl)-2-(trifluoromethyl)phenyl)(prop-2-yn-1-yl)carbamate

Into a 50 mL round-bottom flask was placed tert-butyl (4-(dimethylphosphoryl)-2-(trifluoromethyl)phenyl)carbamate (0.17 g, 0.49 mmol), THF (5 mL). The reaction was stirred at 0° C. NaH (0.02 g, 0.67 mmol) was added to the reaction slowly at 0° C. and stirred for 0.5 h. 3-bromoprop-1-yne (0.49 g, 4.14 mmol) was added to the reaction and stirred at 60° C. overnight. The reaction was quenched by water (50 mL), extracted with EA (2×30 mL). The organic layers were concentrated under vacuum. The mixture purified by flash-C18 eluted with ACN/water (V/V=3/7) to provide the desired product. This resulted in 0.101 g (43.74% yield) of tert-butyl (4-(dimethylphosphoryl)-2-(trifluoromethyl)phenyl)(prop-2-yn-1-yl)carbamate as brown oil. LCMS: m/z=376 [M+1]⁺.

### Step 4. dimethyl(4-(prop-2-yn-1-ylamino)-3-(trifluoromethyl)phenyl)phosphine oxide

Into a 50 mL round-bottom flask was placed tert-butyl (4-(dimethylphosphoryl)-2-(trifluoromethyl)phenyl)(prop-2-yn-1-yl)carbamate (0.10 g, 0.26 mmol), TFA (1 mL), DCM (5 mL). The reaction was stirred at R.T for 0.5 h. The reaction was concentrated under vacuum. The mixture purified by flash-C18 eluted with ACN/water (V/V=3/7) to provide the desired product. This resulted in 0.095 g (crude) of dimethyl(4-(prop-2-yn-1-ylamino)-3-(trifluoromethyl)phenyl)phosphine oxide as off-white solid. LCMS: m/z=276 [M+1]⁺.

### Step 5. (4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-(trifluoromethyl)phenyl)dimethylphosphine oxide (105)

Into a 40 mL vial was placed (3S,4R)-3-fluoro-N-(2-iodo-3-(2,2,2-trifluoroethyl)benzo[b]thiophen-7-yl)-1-meth ylpiperidin-4-amine (0.15 g, 0.32 mmol), dimethyl(4-(prop-2-yn-1-ylamino)-3-(trifluoromethyl)phenyl)phosphine oxide (0.09 g, 0.33 mmol), bis(triphenylphosphine)palladium(II) chloride (0.04 g, 53.83 μmol), CuI (0.01 g, 42.01 μmol), DIEA (0.15 g, 1.19 mmol), Methyl sulfoxide (5 mL). The reaction was stirred at 60° C. for 1 h under nitrogen. The reaction was quenched with water (20 mL), extracted with EA (3×30 mL). The organic layers were combined, washed with brine (50 mL), dried over anhydrous sodium sulfate, filtered and concentrated under vacuum. The residue purified by flash-C18 eluted with ACN/water (V/V=2/3) to provide the desired product. The mixture was purified by preparative HPLC (Mobile Phase A: water (10 mmol/L ammonium hydroxide), Mobile Phase B: ACN; Flow rate: 70 mL/min; Gradient: 15-60-65% B (2-30-40 min); 266 nm; RT: 36.17-36.91 min) to provide the desired product. This resulted in 0.0507 g (25.76% yield) of (4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-(trifluoromethyl)phenyl)dimethylphosphine oxide (105) as white solid. LCMS: m/z=620 [M+1]⁺.

¹H NMR (400 MHz, DMSO-d₆) δ 7.81 (d, J=10.9 Hz, 2H), 7.31-7.13 (m, 3H), 6.78 (d, J=7.7 Hz, 1H), 6.65 (t, J=6.1 Hz, 1H), 5.20 (d, J=8.5 Hz, 1H), 4.80 (d, J=49.4 Hz, 1H), 4.46 (d, J=5.8 Hz, 2H), 3.79 (d, J=11.0 Hz, 2H), 3.72-3.56 (m, 1H), 3.03 (t, J=11.2 Hz, 1H), 2.79 (d, J=10.8 Hz, 1H), 2.18 (s, 4H), 2.12-1.89 (m, 2H), 1.78-1.67 (m, 1H), 1.61 (m, 6H).

### Example 106

### (3-cyclopropoxy-4-((3-(7-(((Z)—-3-fluoro-1-methylpiperidin-4-yl)amino)-3-(2,2,2-trifluoroethyl)benzo[b]thiophen-2-yl)prop-2-yn-1-yl)amino)phenyl)dimethylphosphine oxide (106)

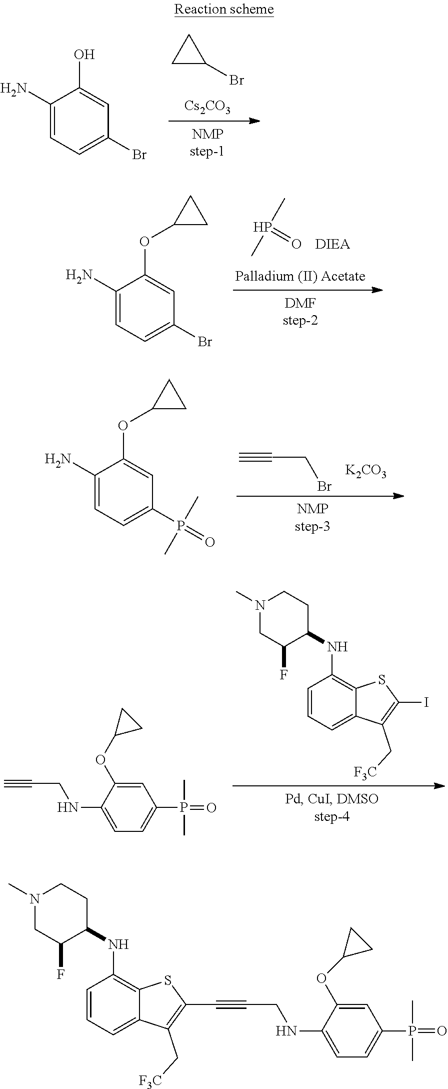

### Experiment Details

### Step 1 4-bromo-2-(fluoromethoxy)-1-nitrobenzene

Into a 40 mL sealed tube was placed 2-amino-5-bromophenol (2.068 g, 10.23 mmol), bromocyclopropane (2.293 g, 18.95 mmol), Cs₂CO₃ (7.317 g, 22.45 mmol), NMP (20 mL). The reaction was stirred at 150° C. or 16 h. The reaction was quenched with water (30 mL). The resulted solution was extracted with EA (3×50 mL), washed with brine (50 mL), dried over anhydrous sodium sulfate, filtered and concentrated under vacuum. The crude product was purified with silica gel column eluted with EA/hexane (v/v=1/4). This resulted in 0.554 g (23.73% yield) of 4-bromo-2-cyclopropoxyaniline as yellow oil. LCMS: m/z=228 [M+1]⁺

### Step 2 (4-amino-3-cyclopropoxyphenyl)dimethylphosphine oxide

Into a 25 ml 3-necked flask was placed 4-bromo-2-cyclopropoxyaniline (0.505 g, 2.21 mmol), Palladium (II) Acetate (0.119 g, 530.04 umol), Xantphos (0.195 g, 337.01 umol), DIEA (0.844 g, 6.53 mmol), DMF (5 mL) The reaction was stirred under nitrogen atmosphere at 130° C. for 0.5 h. Then dimethylphosphine oxide (1 mL) was added at 130° C. The reaction was stirred under nitrogen atmosphere at 130° C. for 1.5 h The reaction mixture was filtered through celite pad and celite pad was washed with ACN (3×20 mL). The filterate was then concentrated under reduced pressure to afford the crude product. The reaction mixture was purified by C18 column eluted with ACN/H₂O (v/v=1/3), filtered and concentrated under vacuum. This resulted in 0.354 g, (70.98% yield) of (4-amino-3-cyclopropoxyphenyl)dimethylphosphine oxide as yellow solid. LCMS: m/z=226 [M+1]⁺.

### Step 3 (3-cyclopropoxy-4-(prop-2-yn-1-ylamino)phenyl)dimethylphosphine oxide

Into a 25 ml 3-necked flask was placed (4-amino-3-cyclopropoxyphenyl)dimethylphosphine oxide (0.336 g, 1.49 mmol), 3-bromoprop-1-yne (0.401 g, 3.37 mmol), K₂CO₃ (0.214 g, 1.54 mmol), KI (0.290 g, 1.74 m mol) NMP (5 mL). The reaction mixture was stirred under nitrogen at 80° C. for 4 h. The reaction mixture was filtered through celite pad and celite pad was washed with ACN (3×20 mL). The filterate was then concentrated under reduced pressure to afford the crude product. The reaction mixture was purified by C18 column eluted with ACN/H₂O (v/v=1/3), filtered and concentrated under vacuum. This resulted in 0.128 g, (32.58% yield) of (3-cyclopropoxy-4-(prop-2-yn-1-ylamino)phenyl)dimethylphosphine oxide as brown solid. LCMS: m/z=264 [M+1]⁺.

### Step 4 (3-cyclopropoxy-4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)phenyl)dimethylphosphine oxide (106)

Into a 10-mL round-bottom flask was placed (3-cyclopropoxy-4-(prop-2-yn-1-ylamino)phenyl)dimethylphosphine oxide (0.127 g, 482.39 umol), (Z)-3-fluoro-N-(2-iodo-3-(2,2,2-trifluoroethyl)benzo[b]thiophen-7-yl)-1-methylpiperidin-4-amine (0.089 g, 188.44 umol), Pd(PPh₃)₂Cl₂ (0.031 g, 43.91 μmol), CuI (0.019 g, 99.76 umol), DIEA (0.100 g, 773.73 umol), methyl sulfoxide (2 mL). The reaction was stirred under nitrogen atmosphere at RT for 3 h. The reaction mixture was filtered through celite pad and celite pad was washed with ACN (3×20 mL). The filterate was then concentrated under reduced pressure to afford the crude product. The c rude product was purified by preparative HPLC (Mobile Phase A: water (0.1% ammonium hydroxide), Mobile Phase B: MeOH; Flow rate: 70 mL/min; Gradient: 35-70-95% B (2-30-60 min); 266 nm; RT: 32.162-34.002) to provide the desired product. This resulted in 0.0303 g (26.46% yield) of (3-cyclopropoxy-4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)phenyl)dimethylphosphine oxide (106) as white solid. LCMS: m/z=608 [M+1]⁺.

¹H NMR (400 MHz, DMSO-d₆) δ 7.43 (d, J=11.8 Hz, 1H), 7.33-7.16 (m, 3H), 6.91-6.71 (m, 2H), 5.94 (t, J=6.3 Hz, 1H), 5.15 (d, J=8.5 Hz, 1H), 4.79 (d, J=49.5 Hz, 1H), 4.32 (d, J=6.3 Hz, 2H), 3.98-3.57 (m, 4H), 3.03 (t, J=11.3 Hz, 1H), 2.79 (d, J=10.9 Hz, 1H), 2.18 (s, 4H), 2.07 (t, J=11.4 Hz, 1H), 1.99-1.92 (m, 1H), 1.71 (d, J=11.5 Hz, 1H), 1.58 (d, J=13.1 Hz, 6H), 0.83-0.66 (m, 4H).

### Example 107 and 133

### 2-(2-(3-((4-(dimethylphosphoryl)-2-methoxyphenyl)amino)prop-1-yn-1-yl)-7-(((Z)-3-fluoro-1-methylpiperidin-4-yl)amino)benzo[b]thiophen-3-yl)acetonitrile (racemic) (107) and 2-(2-(3-((4-(dimethylphosphoryl)-2-methoxyphenyl)amino)prop-1-yn-1-yl)-7-(((E)-3-fluoro-1-methylpiperidin-4-yl)amino)benzo[b]thiophen-3-yl)acetonitrile (racemic) (133)

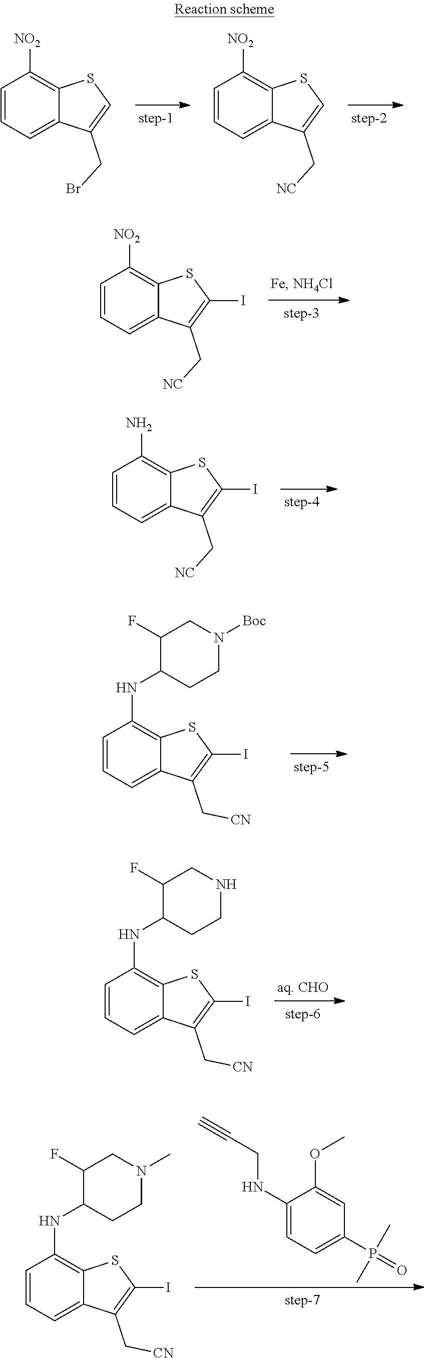

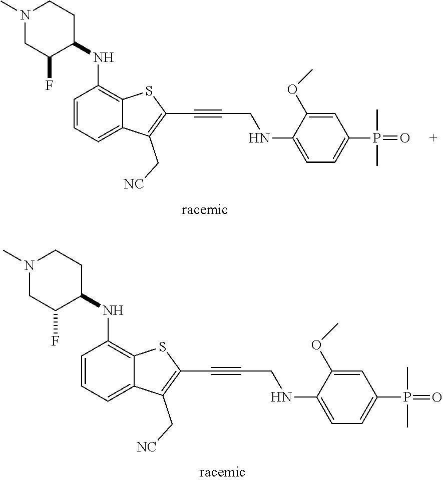

### Experimental Details

### Step 1. 2-(7-nitrobenzo[b]thiophen-3-yl)acetonitrile

Into a 250-mL three necked flask purged and maintained with an inert atmosphere of nitrogen, was placed 3-(bromomethyl)-7-nitrobenzo[b]thiophene (5.11 g, 18.77 mmol), Cyanotrimethylsilane (5.03 g, 50.70 mmol), K₂CO₃ (8.19 g, 59.25 mmol), MeCN (100 mL). The reaction mixture was stirred at 80° C. for 5 h. The resulting solution was added to water (100 mL). The resulting solution was extracted with EA (2×150 mL). The organic layers combined and washed with brine (20 mL), dried over anhydrous Na₂SO₄, the residue was concentrated under vacuum. The residue was applied onto a silica gel column eluted with EA:hexane (v:v=1:1). This resulted in 4.13 g (100.77% yield) of 2-(7-nitrobenzo[b]thiophen-3-yl)acetonitrile as brown solid. LCMS: m/z=219 [M+1]⁺

### Step 2. 2-(2-iodo-7-nitrobenzo[b]thiophen-3-yl)acetonitrile

Into a 500-mL three necked bottle purged and maintained with an inert atmosphere of nitrogen, was placed 2-(7-nitrobenzo[b]thiophen-3-yl)acetonitrile (4.25 g, 19.47 mmol), NIS (10.65 g, 47.33 mmol), Trifluoromethanesulfonic acid (5 mL), Acetic acid (50 mL). The reaction mixture was stirred at room temperature for 5 h. The resulting solution was quenched with water (200 mL). The resulting solution was extracted with E A (2×300 mL). The organic layers combined and washed with brine (100 mL), dried over anhydrous Na 2SO₄, the residue was concentrated under vacuum. The residue was applied onto a silica gel column eluted with EA:hexane (v:v=1:1). This resulted in 5.50 g (82.06% yield) of 2-(2-iodo-7-nitrobenzo[b]thiophen-3-yl)acetonitrile as brown solid. LCMS: m/z=345 [M+1]⁺.

### Step 3. 2-(7-amino-2-iodobenzo[b]thiophen-3-yl)acetonitrile

Into a 500-mL three necked bottle purged and maintained with an inert atmosphere of nitrogen, was placed 2-(2-iodo-7-nitrobenzo[b]thiophen-3-yl)acetonitrile (5.56 g, 16.15 mmol), NH₄Cl (5.29 g, 98.89 mmol), Fe(4.93 g, 88.28 mmol), EtOH (100 mL) and H₂O (20 mL). The reaction mixture was stirred at 80° C. for 2 h. The resulting solution was filtered through a celite pad, and the filtrate extracted with EA (2×200 mL). The organic layers combined and washed with brine (100 mL), dried over anhydrous Na₂SO₄, the residue was concentrated under vacuum. The residue was applied onto a silica gel column eluted with EA:hexane (v/v=1:1). This resulted in 5.12 g (100.87% yield) of 2-(7-amino-2-iodobenzo[b]thiophen-3-yl)acetonitril e as brown solid. LCMS: m/z=315 [M+1]⁺

### Step 4. tert-butyl 4-((3-(cyanomethyl)-2-iodobenzo[b]thiophen-7-yl)amino)-3-fluoropiperidine-1-carboxylate

Into a 250-mL three necked bottle purged and maintained with an inert atmosphere of nitrogen, was placed 2-(7-amino-2-iodobenzo[b]thiophen-3-yl)acetonitrile (5.08 g, 16.17 mmol), tert-butyl 3-fluoro-4-oxopiperidine-1-carboxylate (7.07 g, 32.54 mmol), DMF (100 mL). The reaction mixture was cooled to 0° C. chlorotrimethylsilane (18.92 g, 174.15 mmol) was added and stirred at room temperature for 1 h before BH₃ (1 M in THF, 50 mL, 50 mmol) was added. The reaction mixture was stirred at room temperature for 0.5 h. The resulting solution was quenched with water (100 mL) and extracted with EA (2×100 mL). The organic layers combined and washed with brine (100 mL), dried over anhydrous Na₂SO₄, the residue was concentrated under vacuum. The residue was applied onto a silica gel column eluted with EA:hexane (v:v=1:1). This resulted in 7.70 g (92.39% yield) of tert-butyl 4-((3-(cyanomethyl)-2-iodobenzo[b]thiophen-7-yl)amino)-3-fluoropiperidine-1-carboxylate as yellow oil. LCMS: m/z=516 [M+1]⁺

### Step 5. 2-(7-((3-fluoropiperidin-4-yl)amino)-2-iodobenzo[b]thiophen-3-yl)acetonitrile

Into a 250-mL three necked bottle purged and maintained with an inert atmosphere of nitrogen, was placed tert-butyl 4-((3-(cyanomethyl)-2-iodobenzo[b]thiophen-7-yl)amino)-3-fluoropiperidine-1-carboxylate (5.03 g, 9.75 mmol), TFA (10 mL), DCM (50 mL). The reaction mixture was stirred at room temperature for 1 h. LCMS showed that the reaction was completed. The resulting solution was concentrated under vacuum, quenched with aq·NaHCO₃ (100 mL), and extracted with EA (2×100 mL). The organic layers combined and washed with brine (100 mL), dried over anhydrous Na₂SO₄, the residue was concentrated under vacuum. The residue was applied onto a silica gel column eluted with EA:hexane (v:v=3:1). This resulted in 4.00 g (98.69% yield) of 2-(7-((3-fluoropiperidin-4-yl)amino)-2-iodobenzo[b]thiophen-3-yl)acetonitrile as brown solid. LCMS: m/z=416 [M+1]⁺

### Step 6. 2-(7-((3-fluoro-1-methylpiperidin-4-yl)amino)-2-iodobenzo[b]thiophen-3-yl)acetonitrile

Into a 100-mL three necked bottle purged and maintained with an inert atmosphere of nitrogen, was placed 2-(7-((3-fluoropiperidin-4-yl)amino)-2-iodobenzo[b]thiophen-3-yl)acetonitrile (1.08 g, 2.60 mmol), Formaldehyde (37% in H₂O, 240.00 mg, 29.57 μmol), MeOH (15 mL), Acetic acid (1 mL). The reaction mixture w as stirred at room temperature for 2 h before Sodium cyanoboronhydride (361.00 mg, 8.42 mmol) was added. The reaction mixture was stirred at room temperature for another 12 h. The resulting solution was quenched with water (100 mL) and extracted with EA (2×100 mL). The organic layers combined and washed with brine (100 mL), dried over anhydrous Na₂SO₄, the residue was concentrated under vacuum. The residue was applied onto a silica gel column eluted with EA:hexane (v:v=3:1). This resulted in 1.07 g (95.74% yield) of 2-(7-((3-fluoro-1-methylpiperidin-4-yl)amino)-2-iodobenzo[b]thiophen-3-yl)acetonitrile as yellow solid. LCMS: m/z=430 [M+1]⁺

### Step 7. 2-(2-(3-((4-(dimethylphosphoryl)-2-methoxyphenyl)amino)prop-1-yn-1-yl)-7-(((Z)-3-fluoro-1-methyl piperidin-4-yl)amino)benzo[b]thiophen-3-yl)acetonitrile (racemic) (107) and 2-(2-(3-((4-(dimethylphosphoryl)-2-methoxyphenyl)amino)prop-1-yn-1-yl)-7-(((E)-3-fluoro-1-methylpiperidin-4-yl)amino)benzo[b]thiophen-3-yl)acetonitrile (racemic) (133)

Into a 40-mL three necked bottle purged and maintained with an inert atmosphere of nitrogen, was placed 2-(7-((3-fluoro-1-methylpiperidin-4-yl)amino)-2-iodobenzo[b]thiophen-3-yl)acetonitrile (206.00 mg, 0.48 mmol), (3-methoxy-4-(prop-2-yn-1-ylamino)phenyl)dimethylphosphine oxide (375.00 mg, 1.58 mmol), Pd(PPh₃)₂Cl (36 mg, 0.051 mmol), CuI (93.00 mg, 0.49 mmol), DIEA (324.00 mg, 2.51 mmol), DMSO (5 mL). The reaction mixture was stirred at 50° C. for 3 h. The residue was applied onto a silica gel column eluted with A CN/H₂O (0.01% TFA) (v:v=1:1), Flow rate: 100 mL/min. the residue was concentrated under vacuum. The resulting crude product was further purified by pre-HPLC with MeOH/H₂O (0.1% ammonium hydroxide), F low rate: 70 mL/min; Gradient: 45-80-80% B (2-30-60 min); 266 nm; RT: 28.488-30.210/30.307-31.548 min). This resulted in 42.00 mg (16.25% yield) of 2-(2-(3-((4-(dimethylphosphoryl)-2-methoxyphenyl)amino)prop-1-yn-1-yl)-7-(((Z)-3-fluoro-1-methylpiperidin-4-yl)amino)benzo[b]thiophen-3-yl)acetonitrile(racemic) (107) as off white solid. LCMS: m/z=539 [M+1]⁺

¹H NMR (400 MHz, DMSO-d₆) δ 7.29-7.20 (m, 2H), 7.19-7.08 (m, 2H), 6.96-6.86 (m, 1H), 6.77 (d, J=7.9 Hz, 1H), 6.10 (t, J=6.3 Hz, 1H), 5.64 (d, J=8.6 Hz, 1H), 4.65-4.60 (m, 1H), 4.33 (t, J=21.0 Hz, 2H), 4.16-4.00 (m, 2H), 3.86 (s, 3H), 3.71-3.49 (m, 1H), 3.16-3.01 (m, 1H), 2.69 (d, J=10.3 Hz, 1H), 2.21 (d, J=18.8 Hz, 3H), 2.12-1.86 (m, 4H), 1.61 (s, 3H), 1.57 (s, 3H).

And 21.00 mg (8.12% yield) of 2-(2-(3-((4-(dimethylphosphoryl)-2-methoxyphenyl)amino)prop-1-yn-1-yl)-7-(((E)-3-fluoro-1-methylpiperidin-4-yl)amino)benzo[b]thiophen-3-yl)acetonitrile (racemic) (133) as white solid. LCMS: m/z=539 [M+1]⁺

¹H NMR (400 MHz, DMSO-d₆) δ 7.32 (t, J=7.9 Hz, 1H), 7.28-7.19 (m, 2H), 7.19-7.10 (m, 1H), 6.92-6.83 (m, 1H), 6.81 (d, J=7.8 Hz, 1H), 6.13-6.09 (m, 1H), 5.27 (d, J=8.5 Hz, 1H), 4.79 (d, J=49.7 Hz, 1H), 4.38-4.30 (m, 2H), 4.11 (s, 2H), 3.86 (s, 3H), 3.76-3.56 (m, 1H), 3.03 (t, J=10.7 Hz, 1H), 2.80 (d, J=10.5 Hz, 1H), 2.31-2.15 (m, 4H), 2.08 (t, J=11.2 Hz, 1H), 2.02-1.87 (m, 1H), 1.78-1.65 (m, 1H), 1.59 (d, J=13.2 Hz, 6H).

### Example 108 and 201

### 2-(2-(3-((4-(dimethylphosphoryl)-2-methoxyphenyl)amino)prop-1-yn-1-yl)-7-(((Z)-3-fluoro-1-methylpiperidin-4-yl)amino)benzo[b]thiophen-3-yl)acrylonitrile (racemic) (108) and 2-(2-(3-((4-(dimethylphosphoryl)-2-methoxyphenyl)amino)prop-1-yn-1-yl)-7-(((E)-3-fluoro-1-methylpiperidin-4-yl)amino)benzo[b]thiophen-3-yl)acrylonitrile (racemic) (201)

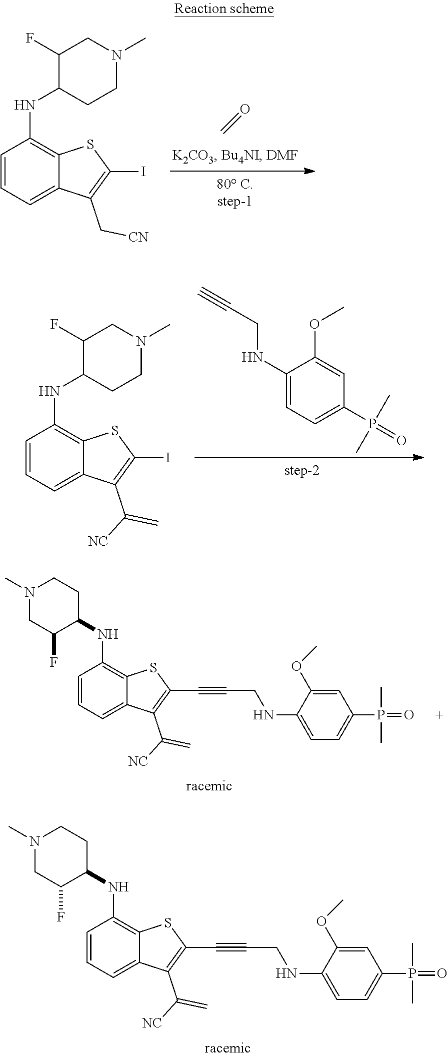

### Experimental Details

### Step 1. 2-(7-((3-fluoro-1-methylpiperidin-4-yl)amino)-2-iodobenzo[b]thiophen-3-yl)acrylonitrile

Into a 100-mL three necked bottle purged and maintained with an inert atmosphere of nitrogen, was placed 2-(7-((3-fluoro-1-methylpiperidin-4-yl)amino)-2-iodobenzo[b]thiophen-3-yl)acetonitrile (320 mg, 0.74 mmol), formaldehyde (129.00 mg, 4.29 mmol), K₂CO₃ (334.00 mg, 2.41 mmol), Tetrabutylammonium iodide (123.00 mg, 0.33 mmol), DMF (10 mL). The reaction mixture was stirred at 80° C. for 2 h. The residue was concentrated under vacuum. The residue was applied onto a C18 column eluted with ACN/H₂O (0.01% TFA) (V:V=1:1), Flow rate: 100 mL/min. This resulted in 302.00 mg (91.80% yield) of 2-(7-((3-fluoro-1-methylpiperidin-4-yl)amino)-2-iodobenzo[b]thiophen-3-yl)acrylonitrile as brown solid. LCMS: m/z=442 [M+1]⁺

### Step 2. 2-(2-(3-((4-(dimethylphosphoryl)-2-methoxyphenyl)amino)prop-1-yn-1-yl)-7-(((Z)-3-fluoro-1-methyl piperidin-4-yl)amino)benzo[b]thiophen-3-yl)acrylonitrile (racemic) (108) and 2-(2-(3-((4-(dimethylphosphoryl)-2-methoxyphenyl)amino)prop-1-yn-1-yl)-7-(((E)-3-fluoro-1-methylpiperidin-4-yl)amino)benzo[b]thiophen-3-yl)acrylonitrile (racemic) (201)

Into a 40-mL three necked bottle purged and maintained with an inert atmosphere of nitrogen, was placed 2-(7-((3-fluoro-1-methylpiperidin-4-yl)amino)-2-iodobenzo[b]thiophen-3-yl)acrylonitrile (301.00 mg, 0.68 mmol), (3-methoxy-4-(prop-2-yn-1-ylamino)phenyl)dimethylphosphine oxide (425.00 mg, 1.79 mmol), Pd(PPh₃)₂Cl (97.00 mg, 0.013 mmol), CuI (83.00 mg, 0.43 mmol), DIEA (545.00 mg, 4.21 mmol), DMSO (5 mL). The reaction mixture was stirred at 50° C. for 3 h. The residue was applied onto a C₁₈ column eluted with ACN/H₂O (0.01% TFA) (v:v=1:1), Flow rate: 100 m/min. the residue was concentrated under vacuum. The resulted crude product was further purified by pre-HPLC with ACN/H₂O (0.1% ammonium hydroxide), Flow rate: 70 mL/min; Gradient: 45-83-87% B (2-30-36 min); 264 nm; RT: 30.80-32.30/33.70-34.98 min). This resulted in 122.00 mg (32.48% yield) of 2-(2-(3-((4-(dimethylphosphoryl)-2-methoxyphenyl)amino)prop-1-yn-1-yl)-7-(((Z)-3-fluoro-1-methylpiperidin-4-yl)amino)benzo[b]thiophen-3-yl)acrylonitrile (racemic) (108) as off white solid. LCMS: m/z=551 [M+1]⁺

¹H NMR (400 MHz, DMSO-d₆) δ 7.32 (t, J=7.9 Hz, 1H), 7.26-7.20 (m, 1H), 7.18-7.10 (m, 2H), 6.90-6.73 (m, 2H), 6.61 (s, 1H), 6.40 (s, 1H), 6.06 (t, J=6.3 Hz, 1H), 5.35 (d, J=8.4 Hz, 1H), 4.91-4.70 (m, 1H), 4.38-4.30 (m, 2H), 3.85 (s, 3H), 3.68-3.60 (m, 1H), 3.04 (t, J=10.9 Hz, 1H), 2.81 (d, J=10.0 Hz, 1H), 2.29 (d, J=12.9 Hz, 1H), 2.19 (s, 3H), 2.10 (t, J=11.4 Hz, 1H), 1.98-1.93 (m, 1H), 1.72 (d, J=10.8 Hz, 1H), 1.59 (d, J=13.2 Hz, 6H).

And 32.00 mg (8.52% yield) of 2-(2-(3-((4-(dimethylphosphoryl)-2-methoxyphenyl)amino)prop-1-yn-1-yl)-7-(((E)-3-fluoro-1-methylpiperidin-4-yl)amino)benzo[b]thiophen-3-yl)acrylonitrile (racemic) (201) as white solid. LCMS: m/z=551 [M+1]⁺

¹H NMR (400 MHz, DMSO-d₆) δ 7.32-7.21 (m, 2H), 7.19-7.12 (m, 2H), 6.89-6.77 (m, 2H), 6.60 (s, 1H), 6.39 (s, 1H), 6.07 (t, J=6.2 Hz, 1H), 5.70 (d, J=8.5 Hz, 1H), 4.72-4.51 (m, 1H), 4.33 (d, J=6.2 Hz, 2H), 3.86 (s, 3H), 3.61 (s, 1H), 3.15-3.05 (m, 1H), 2.69 (d, J=10.6 Hz, 1H), 2.24 (s, 3H), 2.10-1.90 (m, 4H), 1.61 (s, 3H), 1.58 (s, 3H).

### Example 109

### 2-dimethylphosphoryl-5-[3-[7-[((Z)-3-fluoro-1-methyl-4-piperidyl)amino]-3-(2,2,2-trifluoroethyl)benzothiophen-2-yl]prop-2-ynylamino]-4-methoxy-N-methyl-benzamide (racemic) (109)

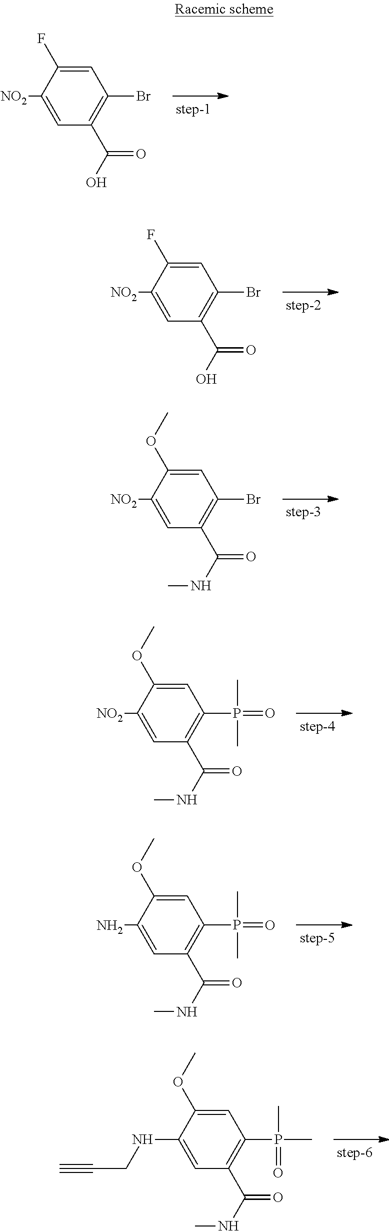

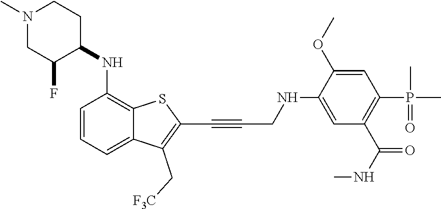

### Experiment Details

### Step 1. Synthesis of 2-bromo-4-methoxy-5-nitro-benzoic acid

Into a 250-mL round-bottom-flask was placed 2-bromo-4-fluoro-5-nitrobenzoic acid (5.740 g, 21.74 mmol), methanol (70 mL). Sodium methanolate (2.551 g, 47.22 mmol) was added slowly after the mixture was cooled to 0° C. Then the mixture was stirred at 10° C. for 4 h. The resulted reaction was adjusted to PH˜3 with HCl in dioxane (4M) and concentrated. The residue was dissolved with water (100 mL) and extracted with EA (100 mL×3). The organic phase was dried over Na₂SO₄ and concentrated under vacuum. This resulted in 5.79 g (96.47% yield) of 2-bromo-4-methoxy-5-nitro-benzoic acid. LCMS: m/z=274 [M−1]−.

### Step 2. Synthesis of 2-bromo-4-methoxy-N-methyl-5-nitro-benzamide

Into a 250-mL round-bottom-flask was placed 2-bromo-4-methoxy-5-nitro-benzoic acid (5.642 g, 20.36 mmol), Methylamine hydrochloride (1.574 g, 23.31 mmol), 2-(7-Aza-1H-Benzotriazole-1-yl)-1,1,3,3-Tetramethyluronium Hexafluorophosphate, (8.795 g, 23.13 mmol), DMF (50 mL), N,N-Diisopropylethylamine (5.999 g, 46.42 mmol). The mixture was stirred at 20° C. for 4 h. The resulted reaction was diluted with water (100 ML) and extracted with EA (100 mL×3). The organic phase was dried over Na₂SO₄ and concentrated under vacuum. The residue was purified by C₁₈ chromatography column eluted with ACN/water (v/v=1/2). This resulted in 2.00 g (33.97% yield) of 2-bromo-4-methoxy-N-methyl-5-nitro-benzamide. LCMS: m/z=287 [M−1]⁻.

### Step 3. Synthesis of 2-dimethylphosphoryl-4-methoxy-N-methyl-5-nitro-benzamide

Into a 40-mL vial was placed 2-bromo-4-methoxy-N-methyl-5-nitro-benzamide (1.735 g, 6.00 mmol), N,N-Dimethylformamide (20 mL), Palladium (II) acetate, (0.248 g, 1.10 mmol), Dimethylbisdiphenylphosphinoxanthene, (1.084 g, 1.87 mmol), N,N-Diisopropylethylamine (4.342 g, 33.59 mmol). The mixture was stirred at 130° C. for 4 h under nitrogen atmosphere. The resulted reaction was purified by C₁₈ chromatography column eluted with ACN/water (v/v=1/2). This resulted in 0.897 g (52.22% yield) of 2-dimethylphosphoryl-4-methoxy-N-methyl-5-nitro-benzamide. LCMS: m/z=285 [M−1]⁻.

### Step 4. Synthesis of 5-amino-2-dimethylphosphoryl-4-methoxy-N-methyl-benzamide

Into a 400-mL vial was placed 2-dimethylphosphoryl-4-methoxy-N-methyl-5-nitro-benzamide (0.855 g, 2.99 mmol), ammonium chloride (2.019 g, 37.74 mmol), iron (1.111 g, 19.89 mmol), ethanol (15 mL), water (10 mL). The mixture was stirred at 50° C. overnight. The resulted reaction was filtered and concentrated under vacuum. The residue was purified by C₁₈ chromatography column eluted with ACN/water (v/v=1/8). This resulted in 0.33 g (43.11% yield) of 5-amino-2-dimethylphosphoryl-4-methoxy-N-methyl-benzamide. LCMS: m/z=255 [M−1]⁻.

### Step 5. Synthesis of 2-dimethylphosphoryl-4-methoxy-N-methyl-5-(prop-2-ynylamino)benzamide

Into a 8-mL vial was placed 5-amino-2-dimethylphosphoryl-4-methoxy-N-methyl-benzamide (0.332 g, 1.29 mmol), N,N-Dimethylformamide (3 mL), N,N-Diisopropylethylamine (0.554 g, 4.28 mmol), sodium iodide (0.045 g, 300.21 μmol), 3-Bromopropyne (0.255 g, 2.14 mmol). The mixture was stirred at 50° C. overnight. The resulted reaction was purified by C₁₈ chromatography column eluted with ACN/water (v/v=1/8). This resulted in 0.10 g (26.22% yield) of 2-dimethylphosphoryl-4-methoxy-N-methyl-5-(prop-2-ynylamino)benzamide. LCMS: m/z=295 [M+1]⁺.

### Step 6. Synthesis of 2-dimethylphosphoryl-5-[3-[7-[((Z)-3-fluoro-1-methyl-4-piperidyl)amino]-3-(2,2,2-trifluoroethyl)benzothiophen-2-yl]prop-2-ynylamino]-4-methoxy-N-methyl-benzamide (racemic) (109)

Into a 4-mL vial was placed 2-dimethylphosphoryl-4-methoxy-N-methyl-5-(prop-2-ynylamino)benzamide (0.046g, 156.31 μmol), (Z)-3-fluoro-N-(2-iodo-3-(2,2,2-trifluoroethyl)benzo[b]thiophen-7-yl)-1-methylpiperidin-4-am me (racemic) (0.071 g, 150.33 μmol), bis(triphenylphosphine)palladium(II) chloride (0.016 g, 22.67 μmol), copper(I) iodide (0.008 g, 42.01 μmol) N,N-Diisopropylethylamine (00.068 g, 526.14 μmol), methyl sulfoxide (1 mL). The mixture was stirred at 50° C. for 3 h. The resulted reaction was diluted with water (30 mL) and extracted with EA (10 mL×3). The organic phase was dried over Na₂SO₄ and concentrated under vacuum. The crude was purified by C₁₈ chromatography column eluted with ACN/water (0.1% ammonium bi carbonate) (v/v=1/1) to give the crude product. Then the crude was concentrated and purified by C₁₈ chromatography column eluted with ACN/water (0.1% formic acid) (v/v=1/1) to give the product. This resulted in 0.038 g (39.58% yield) of 2-dimethylphosphoryl-5-[3-[7-[((Z)-3-fluoro-1-methyl-4-piperidyl)amino]-3-(2,2,2-trifluoroethyl)benzothiophen-2-yl]prop-2-ynylamino]-4-methoxy-N-methyl-benzamide (racemic) (109) as white solid. LCMS: m/z=639 [M+1]⁺.

¹H NMR (400 MHz, DMSO-d6) δ 8.41 (d, J=4.7 Hz, 1H), 7.46 (d, J=12.8 Hz, 1H), 7.27 (t, J=7.7 Hz, 1H), 7.22 (d, J=7.9 Hz, 1H), 6.96 (d, J=4.4 Hz, 1H), 6.78 (d, J=7.5 Hz, 1H), 6.23 (t, J=6.3 Hz, 1H), 5.15 (d, J=8.6 Hz, 1H), 4.85 (s, 1H), 4.73 (s, OH), 4.37 (d, J=6.2 Hz, 2H), 3.86 (s, 3H), 3.80 (t, J=11.1 Hz, 2H), 3.03 (t, J=11.3 Hz, 1H), 2.79 (d, J=11.0 Hz, 1H), 2.75 (d, J=4.5 Hz, 3H), 2.26 (d, J=13.0 Hz, 1H), 2.18 (s, 3H), 2.07 (t, J=11.4 Hz, 1H), 2.00-1.87 (m, 1H), 1.73 (s, 1H), 1.66 (d, J=13.9 Hz, 6H).

### Example 110 and 111

### 4-((3-(4-(((3S,4R)-3-fluoro-1-methylpiperidin-4-yl)amino)-1-(2,2,2-trifluoroethyl)-1H-indol-2-yl)prop-2-yn-1-yl)amino)-3-methoxy-N-methylbenzamide (110) and 4-((3-(4-(((3R,4S)-3-fluoro-1-methylpiperidin-4-yl)amino)-1-(2,2,2-trifluoroethyl)-1H-indol-2-yl)prop-2-yn-1-yl)amino)-3-methoxy-N-methylbenzamide (111)

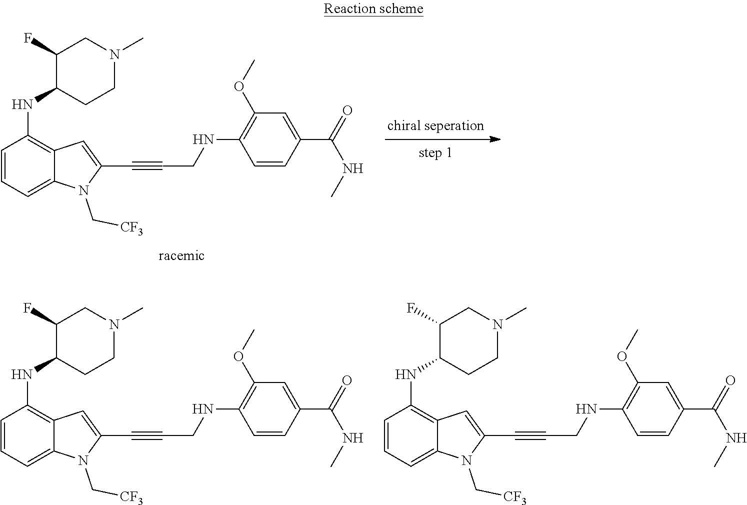

### Experimental Details

### Step 1. 4-((3-(4-(((3S,4R)-3-fluoro-1-methylpiperidin-4-yl)amino)-1-(2,2,2-trifluoroethyl)-1H-indol-2-yl)prop-2-yn-1-yl)amino)-3-methoxy-N-methylbenzamide (110) and 4-((3-(4-(((3R,4S)-3-fluoro-1-methylpiperidin-4-yl)amino)-1-(2,2,2-trifluoroethyl)-1H-indol-2-yl)prop-2-yn-1-yl)amino)-3-methoxy-N-methylbenzamide (111)

The racemic sample 4-((3-(4-(((Z)-3-fluoro-1-methylpiperidin-4-yl)amino)-1-(2,2,2-trifluoroethyl)-1H-indol-2-yl) prop-2-yn-1-yl)amino)-3-methoxy-N-methylbenzamide (300.00 mg, 0.55 mmol) was separated by prep-chiral HPLC (Column:SC, Mobile Phase A: HeX (0.2% IPA.M), Mobile Phase B: EtOH; Flow rate: 20 mL/min; A:B (v:v=60:40); 220 nm; RT: 8.287 min) to provide 107.00 mg (35.66% yield) of 4-((3-(4-(((3R,4S)-3-fluoro-1-methylpiperidin-4-yl)amino)-1-(2,2,2-trifluoroethyl)-1H-indol-2-yl)prop-2-yn-1-yl)amino)-3-methoxy-N-methylbenzamide (111) as white solid. LCMS: m/z=546 [M+1]⁺

¹H NMR (400 MHz, DMSO-d₆) δ 8.09 (d, J=4.3 Hz, 1H), 7.42 (d, J=8.2 Hz, 1H), 7.35 (s, 1H), 7.16 (s, 1H), 7.04-6.92 (m, 1H), 6.77-6.73 (m, 2H), 6.24 (d, J=7.8 Hz, 1H), 5.99-5.96 (m, 1H), 5.48 (d, J=8.6 Hz, 1H), 4.89-4.85 (m, 3H), 4.31 (d, J=6.1 Hz, 2H), 3.84 (s, 3H), 3.71-3.39 (m, 1H), 3.02-2.98 (m, 1H), 2.79-2.76 (m, 4H), 2.32-2.01 (m, 5H), 1.99-1.86 (m, 1H), 1.69 (d, J=11.0 Hz, 1H).

The racemic sample 4-((3-(4-((3-fluoro-1-methylpiperidin-4-yl)amino)-1-(2,2,2-trifluoroethyl)-1H-indol-2-yl)prop-2-yn-1-yl)amino)-3-methoxy-N-methylbenzamide (300.00 mg, 0.55 mmol) was separated by prep-chiral HPLC (Column:SC, Mobile Phase A: HeX (0.2% IPA.M), Mobile Phase B: EtOH; Flow rate: 20 mL/min; A:B (v:v=60:40); 220 nm; RT: 9.396 min) to provide 105.00 mg (35.00% yield) of 4-((3-(4-(((3S,4R)-3-fluoro-1-methylpiperidin-4-yl)amino)-1-(2,2,2-trifluoroethyl)-1H-indol-2-yl)prop-2-yn-1-yl)amino)-3-methoxy-N-methylbenzamide (110) as white solid. LCMS: m/z=546 [M+1]⁺

¹H NMR (400 MHz, DMSO-d₆) δ 8.09 (d, J=4.2 Hz, 1H), 7.42-7.38 (m, 1H), 7.35 (s, 1H), 7.16 (s, 1H), 7.02-6.98 (m, 1H), 6.76-7.72 (m, 2H), 6.24 (d, J=7.7 Hz, 1H), 5.99-5.94 (t, J=6.1 Hz, 1H), 5.48 (d, J=8.5 Hz, 1H), 4.89-4.84 (m, 3H), 4.31 (d, J=6.1 Hz, 2H), 3.84 (s, 3H), 3.60-3.56 (m, 1H), 3.02 (t, J=10.6 Hz, 1H), 2.88-2.65 (m, 4H), 2.32-2.03 (m, 5H), 2.01-1.86 (m, 1H), 1.69 (d, J=10.9 Hz, 1H).

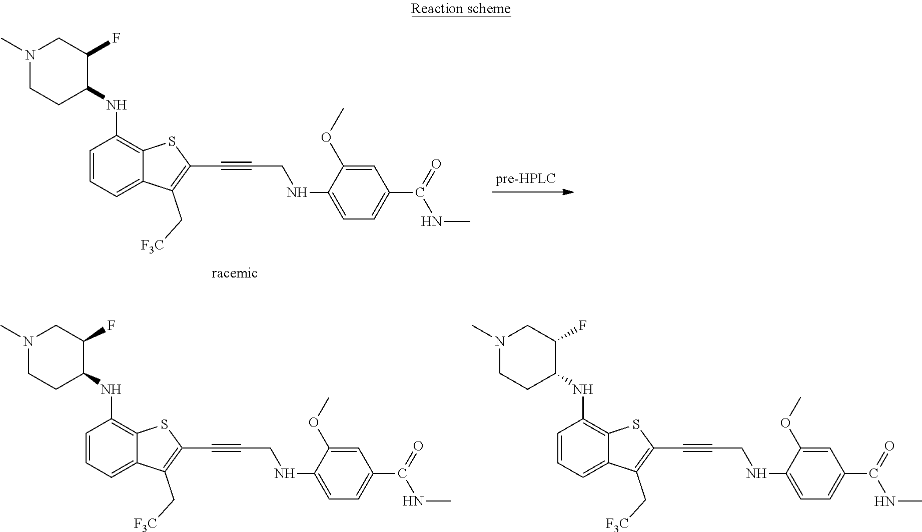

### Experimental Details

### Step 1. 4-((3-(7-(((3R,4S)-3-fluoro-1-methylpiperidin-4-yl)amino)-3-(2,2,2-trifluoroethyl)benzo[b]thiophen-2-yl)prop-2-yn-1-yl)amino)-3-methoxy-N-methylbenzamide (112) and 4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-methoxy-N-methylbenzamide (113)

4-((3-(7-(((Z)-3-fluoro-1-methylpiperidin-4-yl)amino)-3-(2,2,2-trifluoroethyl)benzo[b]thiophen-2-yl)prop-2-yn-1-yl) amino)-3-methoxy-N-methylbenzamide(racemic) (0.31 g, 550.99 μmol) was purified by Chiral HPLC (Column: IG, Mobile Phase A: (HeX:DCM=3:1)(0.2% IPA.M), Mobile Phase B: EtOH; Flow rate: 20 mL/min; A:B (V/V=60/40); 220 nm; RT: 5.427 min) to provide 0.167 g (53.87% yield) of 4-((3-(7-(((3R,4S)-3-fluoro-1-methylpiperidin-4-yl)amino)-3-(2,2,2-trifluoroethyl)benzo[b]thiophen-2-yl)prop-2-yn-1-yl)amino)-3-methoxy-N-methylbenzamide (112) as yellow solid. LCMS: m/z=563 [M+1]⁺.

¹H NMR (400 MHz, DMSO) δ 8.10 (d, J=4.2 Hz, 1H), 7.42 (d, J=8.2 Hz, 1H), 7.35 (s, 1H), 7.27 (t, J=7.8 Hz, 1H), 7.20 (d, J=8.0 Hz, 1H), 6.76 (t, J=7.7 Hz, 2H), 6.00 (t, J=6.3 Hz, 1H), 5.14 (d, J=8.6 Hz, 1H), 4.90-4.71 (m, 1H), 4.34 (d, J=6.2 Hz, 2H), 3.84 (s, 3H), 3.83-3.75 (m, 1H), 3.73-3.56 (m, 1H), 3.03 (t, J=10.1 Hz, 1H), 2.86-2.77 (m, 1H), 2.75 (d, J=4.3 Hz, 3H), 2.32-2.24 (m, 1H), 2.19 (s, 3H), 2.13-2.05 (m, 1H), 2.00-1.90 (m, 1H), 1.77-1.68 (m, 1H), 1.24 (s, 1H). 4-((3-(7-((3-fluoro-1-methylpiperidin-4-yl)amino)-3-(2,2,2-trifluoroethyl)benzo[b]thiophen-2-yl)prop-2-yn-1-yl)amino)-3-methoxy-N-methylbenzamide(racemic) (0.31 g, 550.99 μmol) was purified by Chiral HPLC (Column: IG, Mobile Phase A: (HeX:DCM=3:1)(0.2% IPA.M), Mobile Phase B: EtOH; Flow rate: 20 mL/min; A:B (V/V=60/40); 220 nm; RT: 6.637) to provide 0.165 g (53.22% yield) of 4-((3-(7-(((3S,4R)-3-fluoro-1-meth ylpiperidin-4-yl)amino)-3-(2,2,2-trifluoroethyl)benzo[b]thiophen-2-yl)prop-2-yn-1-yl)amino)-3-methoxy-N-methylbenzamide (113) as yellow solid. LCMS: m/z=563 [M+1]⁺.

¹H NMR (400 MHz, DMSO) δ 8.10 (d, J=4.4 Hz, 1H), 7.42 (d, J=8.0 Hz, 1H), 7.35 (s, 1H), 7.27 (t, J=7.6 Hz, 1H), 7.20 (d, J=7.8 Hz, 1H), 6.76 (t, J=7.5 Hz, 2H), 6.00 (t, J=6.1 Hz, 1H), 5.14 (d, J=8.6 Hz, 1H), 4.90-4.70 (m, 1H), 4.34 (d, J=6.2 Hz, 2H), 3.83 (d, J=7.9 Hz, 3H), 3.82-3.76 (m, 1H), 3.74-3.53 (m, 1H), 3.10-3.00 (m, 1H), 2.80 (d, J=12.3 Hz, 1H), 2.75 (d, J=4.0 Hz, 3H) 2.35-2.25 (m, 1H), 2.19 (s, 3H), 2.08 (d, J=6.8 Hz, 1H), 2.01-1.88 (m, 1H), 1.73 (s, 1H), 1.24 (s, 1H).

### Example 114 and 115

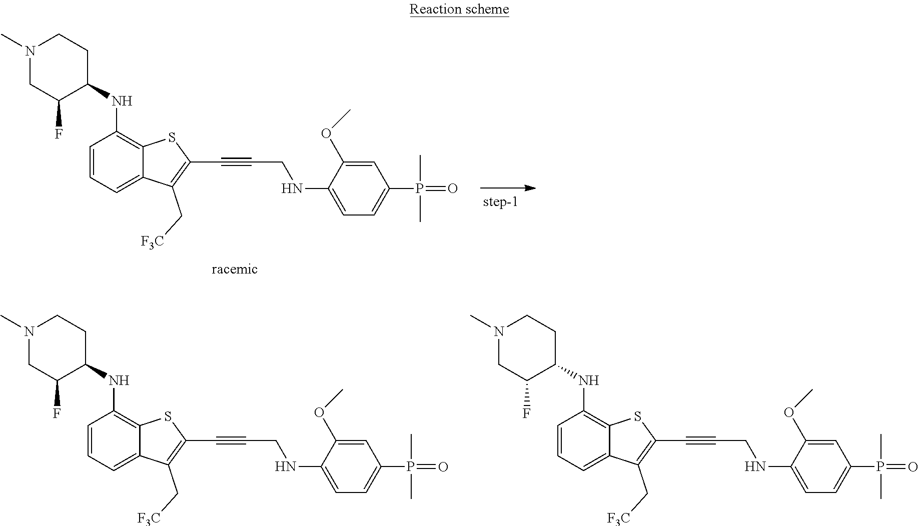 [SMILES: COc1cc(P(C)(C)=O)ccc1NCC#Cc1sc2c(N[C@@H]3CCN(C)C[C@@H]3F)cccc2c1CC(F)(F)F ; COc1cc(P(C)(C)=O)ccc1NCC#Cc1sc2c(N[C@@H]3CCN(C)C[C@@H]3F)cccc2c1CC(F)(F)F ; COc1cc(P(C)(C)=O)ccc1NCC#Cc1sc2c(N[C@H]3CCN(C)C[C@H]3F)cccc2c1CC(F)(F)F]

### Experimental Details

### Step 1. (4-((3-(7-(((3R,4S)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)dimethylphosphine oxide (114) and (4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)dimethylphosphine oxide (115)

The racemic sample (4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)dimethylphosphine oxide (0.326 g, 560.51 umol) was separated by prep-chiral HPLC (Column: 1G, Mobile Phase A: (HeX:DCM=3:1)(0.2% IPA.M), Mobile Phase B: EtOH; Flow rate: 20 m/min; (HeX:DCM=3:1)(0.2% IPA.M):EtOH (v/v=50:50); 220 nm; RT: 6.982 min) to provide 0.160 g (49.07% yield) (4-((3-(7-(((3R,4S)-3-fluoro-1-methylpiperidin-4-yl)amino)-3-(2,2,2-trifluoroethyl) benzo[b]thiophen-2-yl)prop-2-yn-1-yl)amino)-3-methoxyphenyl)dimethylphosphine oxide (114) as white solid. LCMS: m/z=582 [M+1]⁺

¹H NMR (400 MHz, DMSO-d₆) δ 7.33-7.11 (m, 4H), 6.89-6.74 (m, 2H), 6.04 (t, J=6.3 Hz, 1H), 5.14 (d, J=8.5 Hz, 1H), 4.79 (d, J=49.4 Hz, 1H), 4.34 (d, J=6.3 Hz, 2H), 3.81 (d, J=29.7 Hz, 6H), 3.03 (t, J=11.3 Hz, 1H), 2.79 (d, J=11.1 Hz, 1H), 2.28 (d, J=13.0 Hz, 1H), 2.18 (s, 3H), 2.08 (t, J=11.6 Hz, 1H), 2.01-1.89 (m, 1H), 1.72 (d, J=11.2 Hz, 1H), 1.58 (d, J=13.1 Hz, 6H).

The racemic sample (4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)dimethylphosphine oxide (0.326 g, 560.51 umol) was separate d by prep-chiral HPLC (Column:IG, Mobile Phase A: (HeX:DCM=3:1)(0.2% IPA.M), Mobile Phase B: EtOH; Flow rate: 20 m/min; (HeX:DCM=3:1)(0.2% IPA.M):EtOH (v/v=50:50); 220 nm; RT: 9.197 min) to provide 0.144 g (44.17% yield) (4-((3-(7-(((3S,4R)-3-fluoro-1-methylpiperidin-4-yl)amino)-3-(2,2,2-trifluoroethyl) benzo[b]thiophen-2-yl)prop-2-yn-1-yl)amino)-3-methoxyphenyl)dimethylphosphine oxide (115) as white solid. LCMS: m/z=582 [M+1]⁺.

¹H NMR (400 MHz, DMSO-d₆) δ 7.39-7.11 (m, 4H), 6.89-6.72 (m, 2H), 6.04 (t, J=6.3 Hz, 1H), 5.14 (d, J=8.5 Hz, 1H), 4.80 (d, J=49.4 Hz, 1H), 4.34 (d, J=6.4 Hz, 2H), 3.81 (d, J=29.6 Hz, 6H), 3.04 (t, J=11.6 Hz, 1H), 2.80 (d, J=11.2 Hz, 1H), 2.28 (d, J=13.1 Hz, 1H), 2.19 (s, 3H), 2.11 (d, J=11.7 Hz, 1H), 1.96 (td, J=12.2, 3.6 Hz, 1H), 1.72 (d, J=10.7 Hz, 1H), 1.58 (d, J=13.2 Hz, 6H).

### Example 116&134

### (4-((3-(7-(((Z)-3-fluoro-1-methylpiperidin-4-yl)amino)-3-(thiazol-5-yl)benzo[b]thiophen-2-yl)prop-2-yn-1-yl)amino)-3-methoxyphenyl)dimethylphosphine oxide (racemic) (116) and ((4-((3-(7-(((E)-3-fluoro-1-methylpiperidin-4-yl)amino)-3-(thiazol-5-yl)benzo[b]thiophen-2-yl)prop-2-yn-1-yl)amino)-3-methoxyphenyl)dimethylphosphine oxide (racemic) (134)

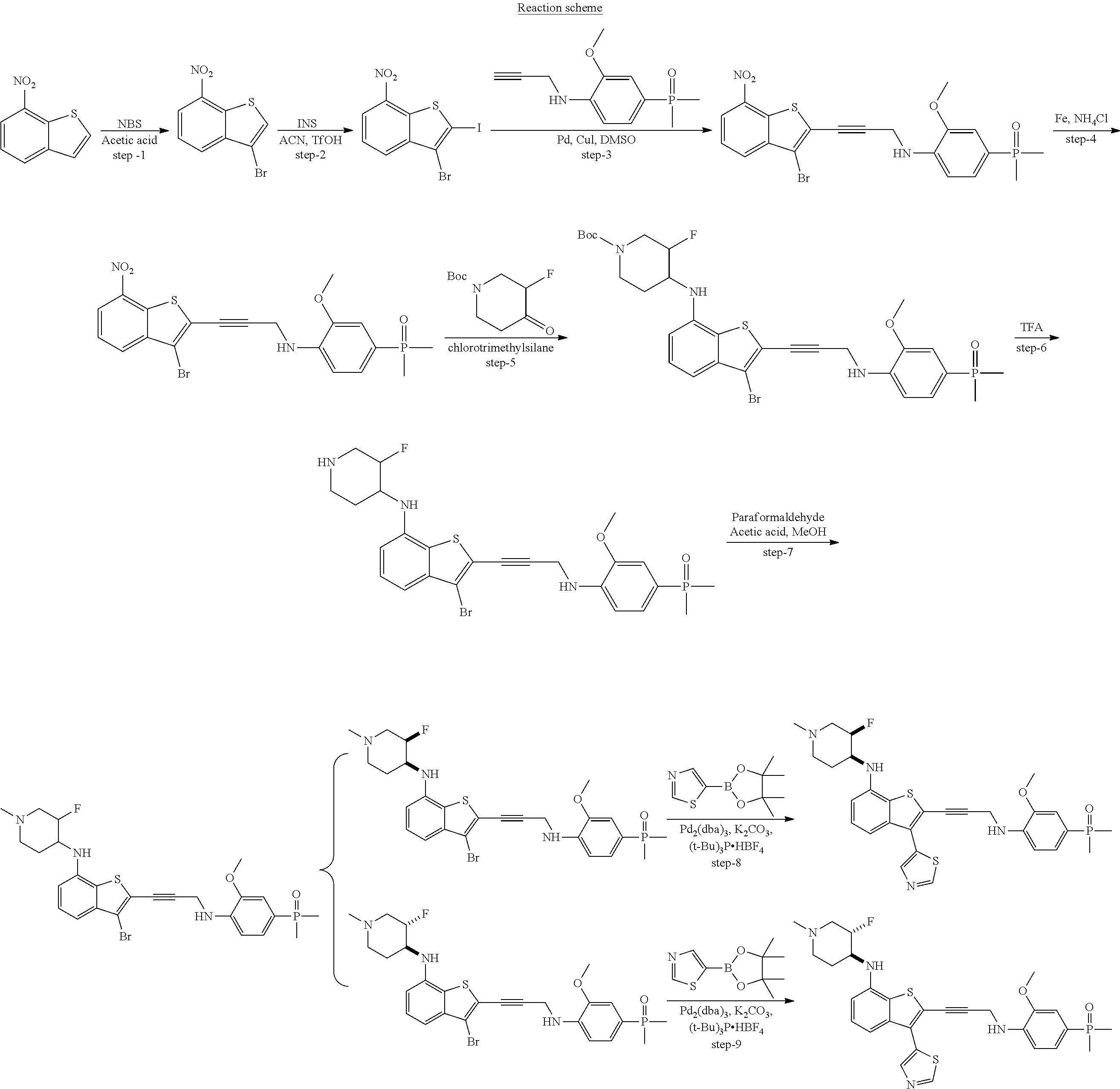

### Experimental Details

### Step 1. 3-bromo-7-nitrobenzo[b]thiophene

Into a 50-mL three necked bottle purged and maintained with an inert atmosphere of nitrogen, was placed 7-nitrobenzo[b]thiophene (1.019 g, 5.68 mmol) and Acetic acid (10 mL). The reaction mixture was stirred at 0° C. Then NBS (1.601 g, 8.99 mmol) was added. The mixture was stirred at 80° C. for 2 h. The crude product purified by washing with HEX/EA (v/v=9/1). This resulted in 0.762 g (51.92% yield) of 3-bromo-7-nitrobenzo[b]thiophene as yellow solid. LCMS: m/z=258 [M+1]⁺

### Step 2. 3-bromo-2-iodo-7-nitrobenzo[b]thiophene

Into a 50-mL three necked bottle purged and maintained with an inert atmosphere of nitrogen, was placed 3-bromo-7-nitrobenzo[b]thiophene (0.751 g, 2.90 mmol) and ACN (5 mL). The reaction mixture was stirred at 0° C. Then NIS (1.30 g, 5377 mmol) was added. The mixture was stirred at 80° C. for 2 h. The mixture was concentrated to afford the crude product. The crude product purified by washing with HEX/EA (v/v=7/1). This resulted in 0.408 g (36.51% yield) of 3-bromo-2-iodo-7-nitrobenzo[b]thiophene as yellow sol id. LCMS: m/z=384 [M+1]⁺

### Step 3. (4-((3-(3-bromo-7-nitrobenzo[b]thiophen-2-yl)prop-2-yn-1-yl)amino)-3-methoxyphenyl)dimethylphosphine oxide

Into a 50-mL three necked bottle and maintained with an inert atmosphere of nitrogen, was placed 3-bromo-2-iodo-7-nitrobenzo[b]thiophene (0.401 g, 1.04 mmol), (3-methoxy-4-(prop-2-yn-1-ylamino)phenyl)dimethylphosphine oxide (0.558 g, 2.35 mmol), CuI (0.071 g, 372.80 umol), Pd(PPh₃)₂Cl₂ (0.140 g, 198.32 umol), DIEA (0.877 g, 6.78 mmol) and DMSO (5 mL). The reaction mixture was stirred at room temperature for 1 h. The reaction mixture was filtered through celite pad and celite pad was washed with ACN (3×20 mL). The filterate was then concentrated under reduced pressure to afford the crude product. The crude product w as purified by C18 column eluted with ACN/H₂O (v/v=2/3), filtered and concentrated under vacuum. This resulted in 0.727 g, (141.11% yield) of (4-((3-(3-bromo-7-nitrobenzo[b]thiophen-2-yl)prop-2-yn-1-yl)amino)-3-methoxyphenyl)dimethylphosphine oxide as yellow solid. LCMS: m/z=493 [M+1]⁺.

### Step 4. (4-((3-(7-amino-3-bromobenzo[b]thiophen-2-yl)prop-2-yn-1-yl)amino)-3-methoxyphenyl)dimethylphosphine oxide

Into a 50 ml 3-necked flask was placed (4-((3-(3-bromo-7-nitrobenzo[b]thiophen-2-yl)prop-2-yn-1-yl)amino)-3-methoxyphenyl)dimethylphosphine oxide (0.547 g, 1.10 mmol), Iron (0.643 g, 11.51 mmol), NH₄Cl (0.787 g, 14.71 mmol), EtOH (10 mL), water (2 mL). The reaction was stirred at 95° C. for 2 h. The reaction mixture was filtered through celite pad and celite pad was washed with methanol (2×30 mL). The filterate w as then concentrated under reduced pressure to afford the product. This resulted in 0.599 g (116.59% yield) of (4-((3-(7-amino-3-bromobenzo[b]thiophen-2-yl)prop-2-yn-1-yl)amino)-3-methoxyphenyl)dimethylphosphine oxide as brown solid. LCMS: m/z=463 [M+1]⁺.

### Step 5. tert-butyl 4-((3-bromo-2-(3-((4-(dimethylphosphoryl)-2-methoxyphenyl)amino)prop-1-yn-1-yl)benzo[b]thiophen-7-yl)amino)-3-fluoropiperidine-1-carboxylate

Into a 25-mL three necked bottle and maintained with an inert atmosphere of nitrogen, (4-((3-(7-amino-3-bromobenzo[b]thiophen-2-yl)prop-2-yn-1-yl)amino)-3-methoxyphenyl)dimethylphosphine oxide (0.550 g, 1.20 m mol), tert-butyl 3-fluoro-4-oxopiperidine-1-carboxylate (0.761 g, 3.50 mmol), chlorotrimethylsilane (1.24 g, 11.42 mmol), DMF (2 mL). The reaction was stirred under nitrogen atmosphere at 0° C. for 1 h. Borane-tetrahydrofuran complex (3.5 ml) was added. The reaction was stirred under nitrogen atmosphere at 0° C. for 1 h. The reaction mixture was concentrated under reduced pressure to afford crude product. The crude product was purified by silica gel column eluted with EA/hexane (v/v=2/3). This resulted in 0.36 g (45.63% yield) of tert-butyl 4-((3-bromo-2-(3-((4-(dimethylphosphoryl)-2-methoxyphenyl)amino)prop-1-yn-1-yl)benzo[b]thiophen-7-yl)amino)-3-fluoropiperidine-1-carboxylate as yellow solid. LCMS: m/z=664 [M+1]⁺.

### Step 6. (4-((3-(3-bromo-7-((3-fluoropiperidin-4-yl)amino)benzo[b]thiophen-2-yl)prop-2-yn-1-yl)amino)-3-methoxyphenyl)dimethylphosphine oxide

Into a 50-mL three necked bottle and maintained with an inert atmosphere of nitrogen, was placed tert-butyl 4-((3-bromo-2-(3-((4-(dimethylphosphoryl)-2-methoxyphenyl)amino)prop-1-yn-1-yl)benzo[b]thiophen-7-yl)amino)-3-fluoropiperidine-1-carboxylate (0.35 g, 526.66 umol), DCM (10 mL) and TFA (2 mL). The reaction mixture was stirred at room temperature for 2 h. The resulting solution was added to sodium carbonate aqueous solution (10 mL) and extracted with EA (3×50 mL). The organic layers combined and washed with brine (20 mL), dried over anhydrous Na₂SO₄, the residue was concentrated under vacuum. This resulted in 0.30 g (100.91% yield) of (4-((3-(3-bromo-7-((3-fluoropiperidin-4-yl)amino)benzo[b]thiophen-2-yl)prop-2-yn-1-yl)amino)-3-methoxyphenyl)dimethylphosphine oxide as yellow solid. LCMS: m/z=564 [M+1]⁺.

### Step 7. (4-((3-(3-bromo-7-(((Z)-3-fluoro-1-methylpiperidin-4-yl)amino)benzo[b]thiophen-2-yl)prop-2-yn-1-yl) amino)-3-methoxyphenyl)dimethylphosphine oxide (racemic) and (4-((3-(3-bromo-7-(((E)-3-fluoro-1-methylpiperidin-4-yl)amino)benzo[b]thiophen-2-yl)prop-2-yn-1-yl)amino)-3-methoxyphenyl)dimethylphosphine ox ide (racemic)

Into a 25-mL round-bottom flask was placed (4-((3-(3-bromo-7-((3-fluoropiperidin-4-yl)amino)benzo[b]thiophen-2-yl)prop-2-yn-1-yl)amino)-3-methoxyphenyl)dimethylphosphine oxide (0.298 g, 527.94 umol), Paraformaldehyde (0.033 g, 1.09 mmol), acetic acid (0.2 mL), methyl alcohol (3 mL) The reaction mixture was stirred at RT for 3 h. Then NaBH₃CN (0.329 g, 7.67 mmol) was added. The reaction mixture was stirred at RT for 4 h. The reaction mixture was filtered through celite pad and celite pad was washed with ACN (3×20 mL). The filterate was then concentrated under reduced pressure to afford the crude product. The reaction mixture was purified by C18 column eluted with ACN/H₂O (v/v=2/3), filtered and concentrated under vacuum. This resulted in 0.141 g, (46.16% yield) of (4-((3-(3-bromo-7-(((E)-3-fluoro-1-methylpiperidin-4-yl)amino)benzo[b]thiophen-2-yl)prop-2-yn-1-yl)amino)-3-methoxyphenyl)dimethylphosphine oxide (racemic) as yellow solid and 0.076 g, (24.88% yield) of (4-((3-(3-bromo-7-(((Z)-3-fluoro-1-methylpiperidin-4-yl)amino)benzo[b]thiophen-2-yl)prop-2-yn-1-yl)amino)-3-methoxyphenyl)dimethylphosphine oxide (racemic). LCMS: m/z=578 [M+1]

### Step 8. (4-((3-(7-(((Z)-3-fluoro-1-methylpiperidin-4-yl)amino)-3-(thiazol-5-yl)benzo[b]thiophen-2-yl)prop-2-yn-1-yl)amino)-3-methoxyphenyl)dimethylphosphine oxide (racemic) (116)

Into a 25-mL three necked bottle and maintained with an inert atmosphere of nitrogen, was placed (4-((3-(3-bromo-7-(((Z)-3-fluoro-1-methylpiperidin-4-yl)amino)benzo[b]thiophen-2-yl)prop-2-yn-1-yl)amino)-3-methoxyphenyl)dimethylphosphine oxide (racemic) (0.0200 g, 34.57 umol), 5-(4,4,5,5-tetramethyl-1,3,2-dioxaborolan-2-yl)thiazole (0.0420 g, 198.96 umol), Pd₂(dba)₃ (0.0090 g, 9.82 umol), (t-Bu)₃PHBF·₄ (0.0061 g, 21.02 umol), K₂CO₃ (0.0392 g, 120.31 umol), dioxane (0.4 mL) and H₂O (0.08 mL). The reaction mixture was stirred at 80° C. for 3 h. The reaction mixture was filtered through celite pad and celite pad was washed with ACN (3×20 mL). The filterate was then concentrated under reduced pressure to afford the crude product. The resulting solution was purified by preparative HPLC (Mobile Phase A: water (0.1% ammonium hydroxide), Mobile Phase B: ACN; Flow rate: 40 mL/min; Gradient: 30-50-50% B (2-30-60 min); 266 nm; RT: 24.725-25.667) to provide the desired product. This resulted in 5.10 mg (25.31% yield) (4-((3-(7-(((Z)-3-fluoro-1-methylpiperidin-4-yl)amino)-3-(thiazol-5-yl)benzo[b]thiophen-2-yl)prop-2-yn-1-yl)amino)-3-methoxyphenyl)dimethylphosphine oxide (racemic) (116) as white solid. LCMS: m/z=583 [M+1]⁺.

### Step 9. ((4-((3-(7-(((E)-3-fluoro-1-methylpiperidin-4-yl)amino)-3-(thiazol-5-yl)benzo[b]thiophen-2-yl)prop-2-yn-1-yl)amino)-3-methoxyphenyl)dimethylphosphine oxide (racemic) (134)

Into a 25-mL three necked bottle and maintained with an inert atmosphere of nitrogen, was placed (4-((3-(3-bromo-7-(((E)-3-fluoro-1-methylpiperidin-4-yl)amino)benzo[b]thiophen-2-yl)prop-2-yn-1-yl)amino)-3-methoxyphenyl)dimethylphosphine oxide (racemic) (0.0201 g, 34.74 umol), 5-(4,4,5,5-tetramethyl-1,3,2-dioxaborolan-2-yl)thiazole (0.0480 g, 193.28 umol), Pd2(dba)3 (0.0087 g, 9.50 umol), (t-Bu)₃P·HBFHBF·₄ (0.0047 g, 19.19 umol), K₂CO₃ (0.0381 g, 116.93 umol), dioxane (0.4 mL) and H₂O (0.08 mL). The reaction mixture was stirred at 80° C. for 3 h. The reaction mixture was filtered through celite pad and celite pad was washed with ACN (3×20 mL). The filterate was then concentrated under reduced pressure to afford the crude product. The resulting solution was purified by preparative HPLC (Mobile Phase A: water (0.1% ammonium hydroxide), Mobile Phase B: ACN; Flow rate: 40 mL/min; Gradient: 30-55-55% B (2-30-60 min); 266 nm; RT: 19.566-20.582) to provide the desired product. This resulted in 4.1000 mg (20.25% yield) (4-((3-(7-(((E)-3-fluoro-1-methylpiperidin-4-yl)amino)-3-(thiazol-5-yl)benzo[b]thiophen-2-yl)prop-2-yn-1-yl)amino)-3-methoxyphenyl)dimethylphosphine oxide (racemic) (134) as white solid. LCMS: m/z=583 [M+1]⁺.

¹H NMR (400 MHz, DMSO-d₆) δ 9.22 (s, 1H), 8.21 (s, 1H), 7.36-7.09 (m, 4H), 6.90-6.73 (m, 2H), 6.05 (t, J=6.4 Hz, 1H), 5.27 (d, J=8.5 Hz, 1H), 4.81 (d, J=49.5 Hz, 1H), 4.31 (d, J=6.4 Hz, 2H), 3.85 (s, 3H), 3.71 (s, 1H), 3.02 (d, J=11.2 Hz, 1H), 2.80 (d, J=11.0 Hz, 1H), 2.28 (d, J=13.1 Hz, 1H), 2.19 (s, 3H), 2.09 (t, J=11.6 Hz, 1H), 1.96 (d, J=11.3 Hz, 1H), 1.73 (d, J=12.4 Hz, 1H), 1.59 (d, J=13.1 Hz, 6H).

### Example 117

### 1-(4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)phospholane 1-oxide (117)

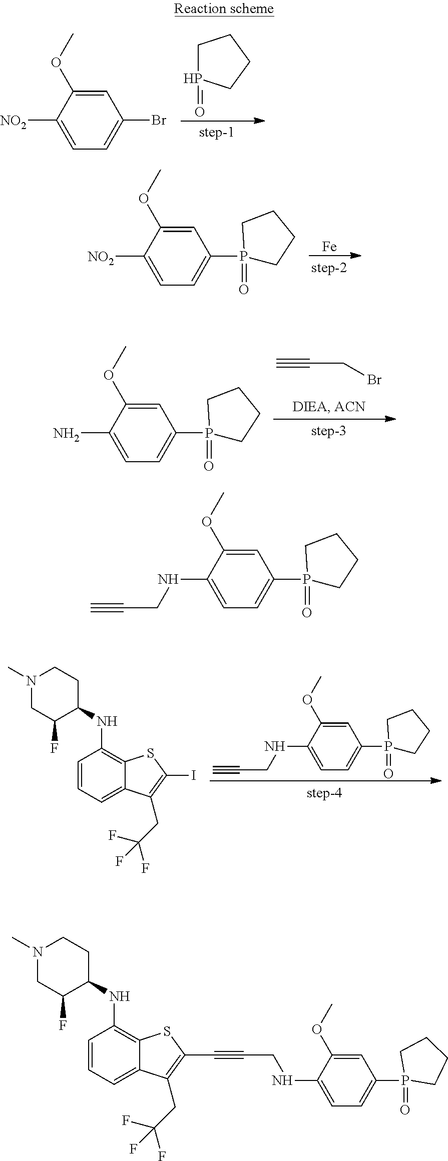

### Experimental Details

### Step 1. 1-(3-methoxy-4-nitrophenyl)phospholane 1-oxide

Into a 100-mL three necked bottle purged and maintained with an inert atmosphere of nitrogen, was placed 4-bromo-2-methoxy-1-nitrobenzene (1.11 g, 4.78 mmol), Palladium (II) acetate (129.00 mg, 0.57 mmol), Dimethylbisdiphenylphosphinoxanthene (559.00 mg, 0.97 mmol), DIEA (1.89 g, 14.62 mmol), DMF (15 mL). The mixture was stirred at 110° C. for 5 min. Then phospholane 1-oxide (492 mg, 4.72 mmol) was added. The mixture was stirred at 110° C. for 3 h. The reaction mixture was filtered. The residue was concentrated under vacuum. The residue was applied onto a C₁₈ column eluted with ACN/H₂O (0.01% TFA) (v:v=1:1), Flow rate: 100 m/min. This resulted in 1.13 g (92.64% yield) of 1-(3-methoxy-4-nitrophenyl)phospholane 1-oxide as yellow solid. LCMS: m/z=256 [M+1]⁺

### Step 2. 1-(4-amino-3-methoxyphenyl)phospholane 1-oxide

Into a 100-mL three necked bottle purged and maintained with an inert atmosphere of nitrogen, was placed 1-(3-methoxy-4-nitrophenyl)phospholane 1-oxide (1.00 g, 3.91 mmol), Fe (790.00 mg, 14.14 mmol), NH₄Cl (92.00 mg, 17.20 mmol), ETOH (25 mL), H₂O (5 mL). The reaction mixture was stirred at 80° C. for 2 h. The reaction mixture was filtered and the filter cake was washed with EtOH (20 mL). The residue was concentrated under vacuum. The residue was applied onto a C₁₈ column eluted with ACN/H₂O (0.01% TFA) (v:v=1:1), Flow rate: 100 m/min. This resulted in 750.00 mg (84.98% yield) of 1-(4-amino-3-methoxyphenyl)phospholane 1-oxide as yellow solid. LCMS: m/z=226 [M+1]⁺

### Step 3. 1-(3-methoxy-4-(prop-2-yn-1-ylamino)phenyl)phospholane 1-oxide

Into a 50-mL three necked bottle purged and maintained with an inert atmosphere of nitrogen, was placed 1-(4-amino-3-methoxyphenyl)phospholane 1-oxide (770.00 mg, 3.41 mmol), 3-bromoprop-1-yne (1.07 g, 8.99 mmol), DIEA (2.01 g, 15.55 mmol), MeCN (20 mL). The reaction mixture was stirred at 80° C. for 5 h. The residue was concentrated under vacuum. The residue was applied onto a C₁₈ column eluted with ACN/H₂O (0.01% TFA) (v:v=1:1), Flow rate: 100 mL/min. This resulted in 428.00 mg (47.55% yield) of 1-(3-m ethoxy-4-(prop-2-yn-1-ylamino)phenyl)phospholane 1-oxide as brown oil. LCMS: m/z=264 [M+1]⁺

### Step 4. 1-(4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)phospholane 1-oxide (117)

Into a 40-mL three necked bottle purged and maintained with an inert atmosphere of nitrogen, was placed (3S,4R)-3-fluoro-N-(2-iodo-3-(2,2,2-trifluoroethyl)benzo[b]thiophen-7-yl)-1-methylpiperidin-4-amine (340.00 mg, 0.64 mmol), Pd(PPh₃)₂Cl₂ (64.00 mg, 0.091 mmol), DIEA (377.00 mg, 2.91 mmol), CuI (50.00 mg, 0.26 mmol), DMSO (5 mL), 1-(3-methoxy-4-(prop-2-yn-1-ylamino)phenyl)phospholane 1-oxide (327.00 mg, 1.24 m mol). The reaction mixture was stirred at 50° C. for 3 h. The residue was applied onto a C₁₈ column elute d with ACN/H₂O (0.01% TFA) (v:v=1:1), Flow rate: 100 mL/min. the residue was concentrated under vacuum. The resulting crude product was further purified by pre-HPLC with ACN/H₂O (0.1% ammonium hydroxide), Flow rate: 70 mL/min; Gradient: 35-70-74% B (2-30-60 min); 266 nm; RT: 30.06-35.97). This resulted in 126.00 mg (32.21% yield) of 1-(4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)phospholane 1-oxide (117) as white solid. LCMS: m/z=608 [M+1]⁺

¹H NMR (400 MHz, DMSO-d₆) δ 7.29-7.24 (m, 1H), 7.23-7.15 (m, 2H), 7.09-7.01 (m, 1H), 6.89-6.81 (m, 1H), 6.78 (d, J=7.6 Hz, 1H), 6.17-6.12 (m 1H), 5.19 (d, J=8.5 Hz, 1H), 4.79 (d, J=49.7 Hz, 1H), 4.33 (t, J=10.9 Hz, 2H), 3.90-3.74 (m, 5H), 3.64 (d, J=27.1 Hz, 1H), 3.03 (t, J=10.9 Hz, 1H), 2.79 (d, J=10.8 Hz, 1H), 2.30-2.14 (m, 4H), 2.08-2.01 (m, 1H), 2.02-1.84 (m, 7H), 1.82-1.67 (m, 3H).

### Example 123

### (4-((3-(7-(((Z)-3-fluoro-1-methylpiperidin-4-yl)amino)-3-(2,2,2-trifluoroethyl)benzo[b]thiophen-2-yl)prop-2-yn-1-yl)oxy)-3-methoxyphenyl)dimethylphosphine oxide(racemic) (123)

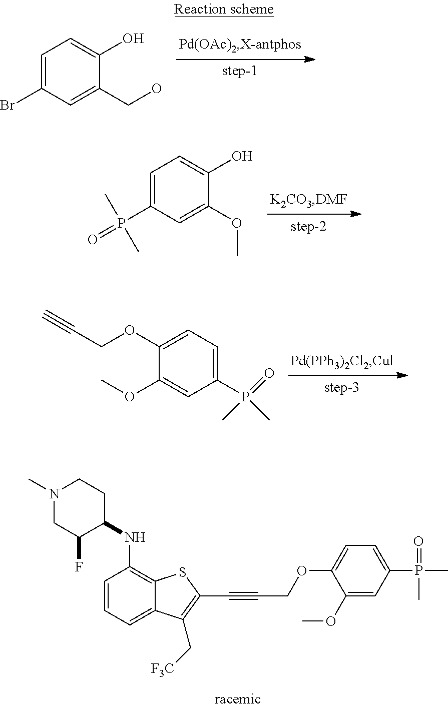

### Experimental Details

### Step 1. (4-hydroxy-3-methoxyphenyl)dimethylphosphine oxide

Into a 25-mL three necked bottle purged and maintained with an inert atmosphere of nitrogen, was placed 4-bromo-2-methoxyphenol (0.506 g, 2.49 mmol), palladium (II) acetate (0.101 g, 449.87 μmol), dimethylbisdiphenylphosphinoxanthene (0.281 g, 485.64 μmol), N,N-dimethylformamide (10 mL). The reaction mixture was stirred at 130° C. Then methylphosphinoylmethane (0.476 g, 6.10 mmol) was added. LCMS showed that the reaction was completed. The residues were purified by C₁₈ chromatography column eluted with ACN/H₂O (0.05% NH₄HCO₃) (v/v=1/3). This resulted in 0.47 g (94.21% yield) of (4-hydroxy-3-methoxyphenyl)dimethylphosphine oxide as white solid. LCMS: m/z=201 [M+1]⁺.

### Step 2. (3-methoxy-4-(prop-2-yn-1-yloxy)phenyl)dimethylphosphine oxide

Into a 10-mL three necked bottle purged and maintained with an inert atmosphere of nitrogen, was placed (4-hydroxy-3-methoxyphenyl)dimethylphosphine oxide (0.239 g, 1.19 mmol), N,N-dimethylformamide (5 m L), potassium carbonate (0.658 g, 4.76 mmol), 3-bromopropyne (0.283 g, 2.38 mmol). The reaction mixture was stirred at 40° C. for 2 h. LCMS showed that the reaction was completed. The residue was concentrated under vacuum and were purified by C₁₈ chromatography column eluted with ACN/H₂O (v/v=1/5). This resulted in 0.22 g (77.35% yield) of (3-methoxy-4-(prop-2-yn-1-yloxy)phenyl)dimethylphosphine oxide as white solid. LCMS: m/z=239 [M+1]⁺.

### Step 3. (4-((3-(7-(((Z)-3-fluoro-1-methylpiperidin-4-yl)amino)-3-(2,2,2-trifluoroethyl)benzo[b]thiophen-2-yl) prop-2-yn-1-yl)oxy)-3-methoxyphenyl)dimethylphosphine oxide(racemic) (123)

Into a 8-mL three necked bottle purged and maintained with an inert atmosphere of nitrogen, was placed (3-methoxy-4-(prop-2-yn-1-yloxy)phenyl)dimethylphosphine oxide (0.059 g, 247.67 μmol), cuprous iodide (0.005 g, 26.25 μmol), triethylamine (0.027 g, 266.83 μmol), methyl sulfoxide (2 mL), (Z)-3-fluoro-N-(2-iodo-3-(2,2,2-trifluoroethyl)benzo[b]thiophen-7-yl)-1-methylpiperidin-4-amine (116.9704 mg, 247.67 μmol), [1,1′-bis(diphenylphosphino)ferrocene]dichloropalladium(II) (0.025 g, 35.41 μmol). The reaction mixture was stirred at 50° C. for 2 h. The reaction was quenched by the addition of water (10 mL) and extracted with EA (2×10 mL). The organic layer was combined, washed with brine (10 mL), dried over anhydrous Na₂SO₄ and concentrated under vacuum. The mixture was purified by preparative HPLC (Mobile Phase A: water (10 mmoL/L ammonium hydroxide), Mobile Phase B: ACN; Flow rate: 40 mL/min; Gradient: 30-55-55% B (2-30-60 min; 260 nm; RT: 23.718-25.705 min). This resulted in 0.027 g (18.71% yield) of (4-((3-(7-(((Z)-3-fluoro-1-methylpiperidin-4-yl)amino)-3-(2,2,2-trifluoroethyl)benzo[b]thiophen-2-yl)prop-2-yn-1-yl)oxy)-3-methoxyphen yl)dimethylphosphine oxide(racemic) (123) as white solid. LCMS: m/z=583 [M+1]⁺.

¹H NMR (400 MHz, DMSO) δ 7.38-7.23 (m, 5H), 6.81 (d, J=7.7 Hz, 1H), 5.27 (d, J=9.0 Hz, 3H), 4.87 (s, 1H), 4.74 (s, 1H), 3.91-3.87 (m, 1H), 3.84 (s, 3H), 3.73-3.60 (m, 1H), 3.07-2.99 (m, 1H) 2.80 (d, J=10.0 Hz, 1H), 2.34-2.25 (m, 1H), 2.18 (s, 3H), 2.12-2.04 (m, 1H), 2.02-1.92 (m, 1H), 1.73 (s, 1H), 1.64 (s, 3H), 1.61 (s, 3H).

### Example 124

### (3-methoxy-4-((3-(7-((3-methyl-3-azabicyclo[3.2.1]octan-8-yl)amino)-3-(2,2,2-trifluoroethyl)benzo[b]thiophen-2-yl)prop-2-yn-1-yl)amino)phenyl)dimethylphosphine oxide (124)

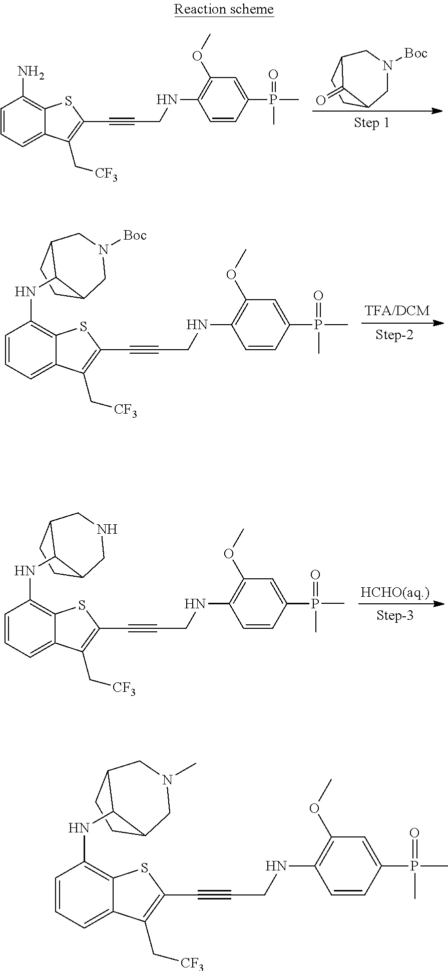

### Experiment Details

### Step 1. tert-butyl 8-((2-(3-((4-(dimethylphosphoryl)-2-methoxyphenyl)amino)prop-1-yn-1-yl)-3-(2,2,2-trifluoroethyl)benzo[b]thiophen-7-yl)amino)-3-azabicyclo[3.2.1]octane-3-carboxylate

Into a 4 mL vail was placed (4-((3-(7-amino-3-(2,2,2-trifluoroethyl)benzo[b]thiophen-2-yl)prop-2-yn-1-yl)amino)-3-methoxyphenyl)dimethylphosphine oxide (0.099 g, 212.24 μmol), tert-butyl 8-oxo-3-azabicyclo[3.2.1]octane-3-carboxylate (0.059 g, 261.89 μmol), PhSiH₃ (0.163 g, 1.51 mmol), Dibutyltin dichloride (0.695 g, 2.29 mmol), THF (1 mL). The reaction was stirred for 3 h at room temperature. LCMS monitored the reaction was complete. The reaction was quenched by MeOH, purified with C₁₈ column (ACN/water v/v=10/1) to afford tert-butyl 8-((2-(3-((4-(dimethylphosphoryl)-2-methoxyphenyl)amino)prop-1-yn-1-yl)-3-(2,2,2-trifluoroethyl)benzo[b]thiophen-7-yl)amino)-3-azabicyclo[3.2.1]octane-3-carboxylate (0.123 g, 182.02 μmol, 85.76% yield) as light brown solid. LCMS: m/z=676[M+1]⁺.

### Step 2. (4-((3-(7-((3-azabicyclo[3.2.1]octan-8-yl)amino)-3-(2,2,2-trifluoroethyl)benzo[b]thiophen-2-yl)prop-2-yn-1-yl)amino)-3-methoxyphenyl)dimethylphosphine oxide

Into a 4 nL flask was placed tert-butyl 8-((2-(3-((4-(dimethylphosphoryl)-2-methoxyphenyl)amino)prop-1-yn-1-yl)-3-(2,2,2-trifluoroethyl)benzo[b]thiophen-7-yl)amino)-3-azabicyclo[3.2.1]octane-3-carboxylate (0.120 g, 177.58 μmol), TFA (0.3 mL), DCM (1.5 mL). The reaction was stirred for 1.5 h at room temperature. LCMS showed the reaction was complete. The reaction quenched with saturated NaHCO₃ (aq.). The solution was diluted with water (2 mL), extracted with EA (2 mL×2). The combined organic layers were washed with water (2 mL) and brine (2 mL) successively, separated, then concentrated with vacuum to afford crude

### (4-((3-(7-((3-azabicyclo[3.2.1]octan-8-yl)amino)-3-(2,2,2-trifluoroethyl)benzo[b]thiophen-2-yl)prop-2-yn-1-yl)amino)-3-methoxyphenyl)dimethylphosphine oxide (0.153 g, 328.84 μmol, 124.78% yield) as an off-white solid. LCMS: m/z=576[M+1]⁺

### Step 3. (3-methoxy-4-((3-(7-((3-methyl-3-azabicyclo[3.2.1]octan-8-yl)amino)-3-(2,2,2-trifluoroethyl)benzo[b]thiophen-2-yl)prop-2-yn-1-yl)amino)phenyl)dimethylphosphine oxide (124)

Into a 4 mL vail was placed (4-((3-(7-((3-azabicyclo[3.2.1]octan-8-yl)amino)-3-(2,2,2-trifluoroethyl)benzo[b]thiophen-2-yl)prop-2-yn-1-yl)amino)-3-methoxyphenyl)dimethylphosphine oxide (0.121 g, 210.21 μmol), formaldehyde (0.026 g, 303.07 μmol), sodium cyanoboronhydride (0.072 g, 1.68 mmol), MeOH (1 mL), acetic acid (0.01 mL). The reaction was stirred for 1.5 h at room temperature. LCMS showed the reaction was complete. The residue was purified with prep-HPLC (Mobile Phase A: water (formic acid), Mobile Phase B: A CN; Flow rate: 70 mL/min; Gradient: 60-85-100% B (2-30-50 min); 266 nm; RT: 39.038-41.792 min) to afford (3-methoxy-4-((3-(7-((3-methyl-3-azabicyclo[3.2.1]octan-8-yl)amino)-3-(2,2,2-trifluoroethyl)benzo[b]thiophen-2-yl)prop-2-yn-1-yl)amino)phenyl)dimethylphosphine oxide (124) (0.030 g, 50.88 μmol, 24.20% yield) as a white solid. LCMS: m/z=590[M−1]⁺

¹H NMR (400 MHz, DMSO-d₆) δ 7.30-7.12 (m, 4H), 6.89-6.81 (m, 1H), 6.71 (d, J=7.7 Hz, 1H), 6.08 (t, J=6.4 Hz, 1H), 5.20 (d, J=3.6 Hz, 1H), 4.34 (d, J=6.3 Hz, 2H), 3.91-3.74 (m, 5H), 3.54-3.44 (m, 1H), 2.43 (d, J=10.4 Hz, 2H), 2.31 (d, J=18.9 Hz, 4H), 2.13 (s, 3H), 1.72 (d, J=4.5 Hz, 4H), 1.58 (d, J=13.1 Hz, 6H).

### Example 125&126&127&128

### (4-((3-(7-(((3S,4R)-1,3-dimethylpiperidin-4-yl)amino)-3-(2,2,2-trifluoroethyl)benzo[b]thiophen-2-yl)prop-2-yn-1-yl)amino)-3-methoxyphenyl)dimethylphosphine oxide (126); (4-((3-(7-(((3R,4S)-1,3-dimethylpiperidin-4-yl)amino)-3-(2,2,2-trifluoroethyl)benzo[b]thiophen-2-yl)prop-2-yn-1-yl)amino)-3-methoxyphenyl)dimethylphosphine oxide (125); (4-((3-(7-(((3S,4S)-1,3-dimethylpiperidin-4-yl)amino)-3-(2,2,2-trifluoroethyl)benzo[b]thiophen-2-yl)prop-2-yn-1-yl)amino)-3-methoxyphenyl)dimethylphosphine oxide (127); (4-((3-(7-(((3R,4R)-1,3-dimethylpiperidin-4-yl)amino)-3-(2,2,2-trifluoroethyl)benzo[b]thiophen-2-yl)prop-2-yn-1-yl)amino)-3-methoxyphenyl)dimethylphosphine oxide (128)

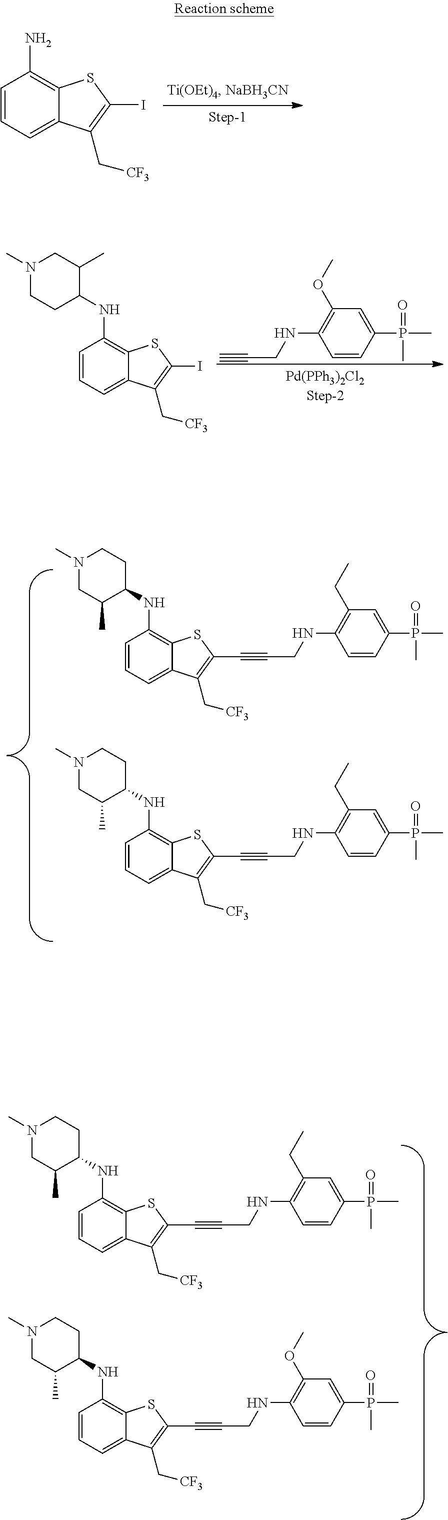

### Experiment Details

### Step 1. N-(2-iodo-3-(2,2,2-trifluoroethyl)benzo[b]thiophen-7-yl)-1,3-dimethylpiperidin-4-amine

Into a 40 mL vail was placed 2-iodo-3-(2,2,2-trifluoroethyl)benzo[b]thiophen-7-amine (1.050 g, 2.94 mmol), 1,3-dimethylpiperidin-4-one (0.725 g, 5.70 mmol), Titanium ethoxide (1.300 g, 5.70 mmol). The reaction was heated to 130° C. for 3 h. Then the reaction was cooled to room temperature naturally, sodium cyanoborohydride (1.12 g, 18.11 mmol), MeOH (5 mL), acetic acid (0.01 mL) was added. The reaction was continue stirred overnight. LCMS showed the reaction was complete. The reaction was quenched with water (10 mL), extracted with EA (10 mL×2). The combined organic layer was wash with water (10 mL) and brine (10 mL), concentrated under vacuum. The residue was purified with Silica-gel column, eluted with n-Heptane/EA (v/v=1/2) to afford N-(2-iodo-3-(2,2,2-trifluoroethyl)benzo[b]thiophen-7-yl)-1,3-dimethylpiperidin-4-amine (1.120 g, 2.39 mmol, 81.34% yield) as light yellow solid. LCMS: m/z=469[M+1]⁺.

### Step 2. (4-((3-(7-(((3S,4R)-1,3-dimethylpiperidin-4-yl)amino)-3-(2,2,2-trifluoroethyl)benzo[b]thiophen-2-yl)prop-2-yn-1-yl)amino)-3-methoxyphenyl)dimethylphosphine oxide (126); (4-((3-(7-(((3R,4S)-1,3-dimethylpiperidin-4-yl)amino)-3-(2,2,2-trifluoroethyl)benzo[b]thiophen-2-yl)prop-2-yn-1-yl)amino)-3-methoxyphenyl)dimethylphosphine oxide (125); (4-((3-(7-(((3S,4S)-1,3-dimethylpiperidin-4-yl)amino)-3-(2,2,2-trifluoroethyl)benzo[b]thiophen-2-yl)prop-2-yn-1-yl)amino)-3-methoxyphenyl)dimethylphosphine oxide (127); (4-((3-(7-(((3R,4R)-1,3-dimethylpiperidin-4-yl)amino)-3-(2,2,2-trifluoroethyl)benzo[b]thiophen-2-yl)prop-2-yn-1-yl)amino)-3-methoxyphenyl)dimethylphosphine oxide (128)

Into a 25 mL flask purged and maintained with nitrogen atmosphere was placed N-(2-iodo-3-(2,2,2-trifluoro ethyl)benzo[b]thiophen-7-yl)-1,3-dimethylpiperidin-4-amine (0.050 g, 2.24 mmol), (3-methoxy-4-(prop-2-yn-1-ylamino)phenyl)dimethylphosphine oxide (0.567 g, 2.39 mmol), Pd(PPh₃)₂Cl₂ (0.164 g, 232.32 μmol), CuI (0.044 g, 231.03 μmol), TEA (0.399 g, 3.94 mmol), DMF (5 mL), and stirred for 3 h at room temperature. LCMS monitored that the reaction was complete. The reaction was quenched with water (10 mL), extracted with EA (5 mL×2). The combined organic layers were washed with water (4 mL) and brine (4 mL) successively, separated, then concentrated with vacuum. The residue was pre-purified with Silica-gel column, eluted with DCM/MeOH (v/v=3/1). Then separated with Chiral HPLC (Column: SA, Mobile Phase A: n-Hexane, Mobile Phase B: EtOH; Flow rate: 20 mL/min; v/v=60/40; 200 nm; RT: 6.293, 7.226, 8.826), to afford (4-((3-(7-(((Z)-1,3-dimethylpiperidin-4-yl)amino)-3-(2,2,2-trifluoroethyl)benzo[b]thiophen-2-yl)prop-2-yn-1-yl)amino)-3-methoxyphenyl)dimethylphosphine oxide (racemic); (4-((3-(7-(((3S,4S)-1,3-dimethylpiperidin-4-yl)amino)-3-(2,2,2-trifluoroethyl)benzo[b]thiophen-2-yl)prop-2-yn-1-yl)amino)-3-methoxyphenyl)dimethylphosphine oxide (127) (0.048 g, 83.10 μmol, 3.71% yield) as a white solid; LCMS: m/z=578 [M+1]⁺.

¹H NMR (400 MHz, DMSO-d₆) δ 7.27-7.11 (m, 4H), 6.84 (dd, J=8.0, 2.9 Hz, 1H), 6.70 (d, J=7.8 Hz, 1H), 6.05 (t, J=6.3 Hz, 1H), 5.02 (s, 1H), 4.34 (d, J=6.3 Hz, 2H), 3.81 (d, J=32.4 Hz, 5H), 3.59 (d, J=9.6 Hz, 1H), 2.66 (s, 2H), 2.29-1.95 (m, 7H), 1.92-1.80 (m, 1H), 1.58 (d, J=13.1 Hz, 6H), 0.90 (d, J=6.9 Hz, 3H).

(4-((3-(7-(((3R,4R)-1,3-dimethylpiperidin-4-yl)amino)-3-(2,2,2-trifluoroethyl)benzo[b]thiophen-2-yl)prop-2-yn-1-yl)amino)-3-methoxyphenyl)dimethylphosphine oxide (128) (0.056 g, 96.95 μmol, 4.32% yield) as a white solid. LCMS: m/z=578 [M+1]⁺.

¹H NMR (400 MHz, DMSO-d₆) δ 7.27-7.11 (m, 4H), 6.87-6.82 (m, 1H), 6.70 (d, J=7.8 Hz, 1H), 6.05 (t, J=6.3 Hz, 1H), 5.02 (s, 1H), 4.34 (d, J=6.3 Hz, 2H), 3.81 (d, J=32.2 Hz, 5H), 3.59 (s, 1H), 2.67 (s, 2H), 2.37-1.97 (m, 7H), 1.92-1.81 (m, 1H), 1.58 (d, J=13.1 Hz, 6H), 0.90 (d, J=6.8 Hz, 3H).

The racemic compounds were separated with Chiral HPLC (Column: IG, Mobile Phase A: n-Hexane, Mobile Phase B: EtOH; Flow rate: 20 mL/min; v/v=50/50; 200 nm; RT: 8.091, 10.459) to afford (4-((3-(7-(((3R,4S)-1,3-dimethylpiperidin-4-yl)amino)-3-(2,2,2-trifluoroethyl)benzo[b]thiophen-2-yl)prop-2-yn-1-yl)amino)-3-methoxyphenyl)dimethylphosphine oxide (125) (0.062 g, 107.33 μmol, 4.79% yield) as a white solid, LCMS: m/z=578 [M+1]⁺.

¹H NMR (400 MHz, DMSO-d₆) δ 7.25-7.09 (m, 4H), 6.88-6.81 (m, 1H), 6.73-6.63 (m, 1H), 6.05 (t, J=6.4 Hz, 1H), 5.25 (d, J=9.1 Hz, 1H), 4.34 (d, J=6.4 Hz, 2H), 3.82 (d, J=25.5 Hz, 5H), 3.01 (d, J=10.2 Hz, 1H), 2.79 (d, J=11.7 Hz, 2H), 2.16 (s, 3H), 2.03-1.84 (m, 4H), 1.69 (t, J=11.2 Hz, 1H), 1.58 (d, J=13.1 Hz, 7H), 0.87 (d, J=6.4 Hz, 3H).

(4-((3-(7-(((3S,4R)-1,3-dimethylpiperidin-4-yl)amino)-3-(2,2,2-trifluoroethyl)benzo[b]thiophen-2-yl)prop-2-yn-1-yl) amino)-3-methoxyphenyl)dimethylphosphine oxide (126) (0.063 g, 109.06 μmol, 4.86% yield) as a white solid. LCMS: m/z=578 [M+1]⁺.

¹H NMR (400 MHz, DMSO-d₆) δ 7.25-7.07 (m, 4H), 6.84 (dd, J=8.5, 2.8 Hz, 1H), 6.65 (d, J=8.0 Hz, 1H), 6.05 (t, J=6.3 Hz, 1H), 5.24 (d, J=9.0 Hz, 1H), 4.34 (d, J=6.5 Hz, 2H), 3.82 (d, J=25.9 Hz, 5H), 3.05-2.96 (m, 1H), 2.75 (d, J=12.6 Hz, 2H), 2.15 (d, J=7.9 Hz, 3H), 2.01-1.82 (m, 4H), 1.68 (t, J=10.7 Hz, 1H), 1.58 (d, J=13.1 Hz, 6H), 0.87 (d, J=6.7 Hz, 3H).

### Example 129

### 1-(tert-butyl)-N-(3-(7-(((Z)-3-fluoro-1-methylpiperidin-4-yl)amino)-3-(2,2,2-trifluoroethyl)benzo[b]thiophen-2-yl)prop-2-yn-1-yl)-1H-pyrazole-4-carboxamide (racemic) (129)

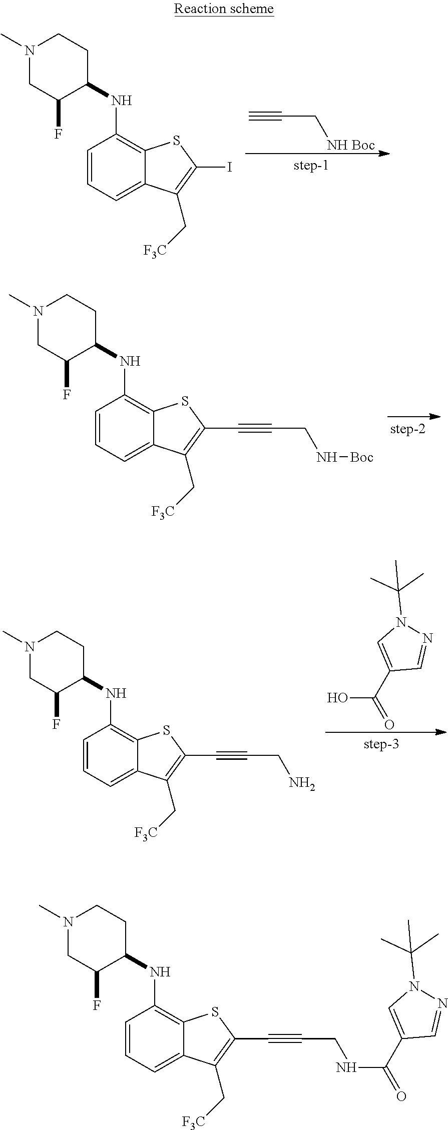

### Experiment Details

### Step 1. Synthesis of tert-butyl (3-(7-(((Z)-3-fluoro-1-methylpiperidin-4-yl)amino)-3-(2,2,2-trifluoroethyl)benzo[b]thiophen-2-yl)prop-2-yn-1-yl)carbamate (racemic)

Into a 20-mL vial was placed (Z)-3-fluoro-N-(2-iodo-3-(2,2,2-trifluoroethyl)benzo[b]thiophen-7-yl)-1-methylpiperidin-4-amine (racemic) (0.202 g, 427.71 μmol), Methyl sulfoxide (3 mL), tert-butyl prop-2-yn-1-ylcarbamate (0.135 g, 869.88 μmol), Bis(triphenylphosphine)palladium(II) chloride (0.034 g, 48.163 μmol), Copper(I) iodide (0.030 g, 157.52 μmol), N,N-Diisopropylethylamine (0.188 g, 1.45 mmol). The mixture was stirred at 90° C. under nitrogen atmosphere for 3 h. The resulted reaction was diluted with water (30 mL) and extracted with EA (10 mL×3). The organic phase was dried over Na₂SO₄ and concentrated under vacuum. This resulted in 0.385 g (crude) of tert-butyl (3-(7-(((Z)-3-fluoro-1-methylpiperidin-4-yl)amino)-3-(2,2,2-trifluoroethyl)benzo[b]thiophen-2-yl)prop-2-yn-1-yl)carbamate. LCMS: m/z=500 [M+1]⁺.

### Step 2. Synthesis of (Z)—N-(2-(3-aminoprop-1-yn-1-yl)-3-(2,2,2-trifluoroethyl)benzo[b]thiophen-7-yl)-3-fluoro-1-methylpiperidin-4-amine (racemic)

Into a 20-mL vial was placed tert-butyl N-[3-[7-[(3-fluoro-1-methyl-4-piperidyl)amino]-3-(2,2,2-trifluoroethyl) benzothiophen-2-yl]prop-2-ynyl]carbamate (racemic) (0.245 g, 490.43 μmol), Dichloromethane (2 mL), Trifluoroacetic acid (400 μL). The resulted reaction was concentrated. Then residue was dissolved with water (10 mL). Then adjusted PH ˜8 with NaHCO₃ (aq) and extracted with EA (10 mL×3). The organic phase was dried over Na₂SO₄ and concentrated under vacuum. This resulted in 0.134 g (crude) of (Z)—N-(2-(3-aminoprop-1-yn-1-yl)-3-(2,2,2-trifluoroethyl)benzo[b]thiophen-7-yl)-3-fluoro-1-methylpiperidin-4-amine (racemic). LCMS: m/z=400 [M+1]⁺.

### Step 3. 1-(tert-butyl)-N-(3-(7-(((Z)-3-fluoro-1-methylpiperidin-4-yl)amino)-3-(2,2,2-trifluoroethyl)benzo[b]thiophen-2-yl)prop-2-yn-1-yl)-1H-pyrazole-4-carboxamide (racemic) (129)

Into a 40-mL vial was placed (Z)—N-(2-(3-aminoprop-1-yn-1-yl)-3-(2,2,2-trifluoroethyl)benzo[b]thiophen-7-yl)-3-fluoro-1-methylpiperidin-4-amine (racemic) (0.065 g, 162.72 μmol), N,N-Dimethylformamide (1 mL), 1-(ter t-butyl)-1H-pyrazole-4-carboxylic acid (0.030 g, 178.37 μmol), 2-(7-Aza-1H-Benzotriazole-1-yl)-1,1,3,3-Tetramethyluronium Hexafluorophosphate (0.073 g, 191.99 μmol), N,N-Diisopropylethylamine (0.051 g, 394.61 μm ol). The mixture was stirred at 20° C. for 4 h. The resulted reaction was purified by C₁₈ chromatography column eluted with ACN/water (0.1% ammonium bicarbonate) (v/v=1/1) to give the product. This resulted in 0.014 g (15.63% yield) of 1-(tert-butyl)-N-(3-(7-(((Z)-3-fluoro-1-methylpiperidin-4-yl)amino)-3-(2,2,2-trifluoroethyl)benzo[b]thiophen-2-yl)prop-2-yn-1-yl)-1H-pyrazole-4-carboxamide (racemic) (129) as white solid. LCMS: m/z=550 [M+1]⁺.

¹H NMR (400 MHz, DMSO-d6) δ 8.64 (t, J=5.5 Hz, 1H), 8.31 (s, 1H), 7.89 (s, 1H), 7.27 (dt, J=16.3, 7.9 Hz, 2H), 6.80 (d, J=7.6 Hz, 1H), 5.17 (d, J=8.6 Hz, 1H), 4.87 (s, 1H), 4.74 (s, 1H), 4.37 (d, J=5.5 Hz, 2H), 3.95 (d, J=11.0 Hz, 1H), 3.89 (d, J=11.1 Hz, 1H), 3.03 (t, J=11.3 Hz, 1H), 2.80 (d, J=11.2 Hz, 1H), 2.27 (d, J=13.0 Hz, 1H), 2.18 (s, 4H), 2.08 (t, J=11.4 Hz, 1H), 2.02-1.88 (m, 1H), 1.73 (d, J=11.7 Hz, 1H), 1.53 (s, 9H).

### Example 130

### 1-tert-butyl-N-[3-[7-[((Z)-3-fluoro-1-methyl-4-piperidyl)amino]-3-(2,2,2-trifluoroethyl)benzothiophen-2-yl]prop-2-ynyl]pyrrole-3-carboxamide (racemic) (130)

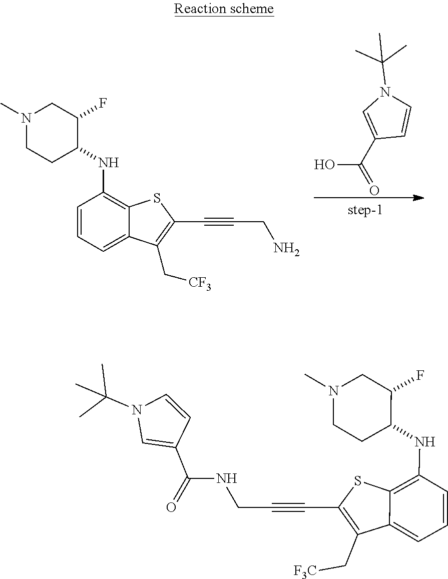

### Experiment Details

### Step 1. Synthesis of 1-tert-butyl-N-[3-[7-[((Z)-3-fluoro-1-methyl-4-piperidyl)amino]-3-(2,2,2-trifluoroethyl) benzothiophen-2-yl]prop-2-ynyl]pyrrole-3-carboxamide (racemic) (130)

Into a 40-mL vial was placed (Z)—N-(2-(3-aminoprop-1-yn-1-yl)-3-(2,2,2-trifluoroethyl)benzo[b]thiophen-7-yl)-3-fluoro-1-methylpiperidin-4-amine (racemic) (0.065 g, 162.72 μmol), N,N-Dimethylformamide (1 mL), 1-(ter t-butyl)-1H-pyrazole-4-carboxylic acid (0.059 g, 147.70 μmol), N,N-Dimethylformamide (1 mL), 2-(7-Aza-1H-Benzotriazole-1-yl)-1,1,3,3-Tetramethyluronium Hexafluorophosphate (0.069 g, 181.47 μmol), N,N-Diisopropylethylamine (0.043 g, 332.71 μmol), -(tert-butyl)-1H-pyrrole-3-carboxylic acid (0.031 g, 185.40 μmol). The mixture was stirred at 20° C. for 4 h. The resulted reaction was purified by C₁₈ chromatography column eluted with ACN/water (0.1% ammonium bicarbonate) (v/v=1/1) to give the product. This resulted in 0.0029 g (3.58% yield) of 1-tert-butyl-N-[3-[7-[((Z)-3-fluoro-1-methyl-4-piperidyl)amino]-3-(2,2,2-trifluoroethyl)benzothiophen-2-yl]prop-2-ynyl]pyrrole-3-carboxamide (racemic) (130) as white solid. LCMS: m/z=549 [M+1]⁺.

¹H NMR (400 MHz, DMSO-d₆) δ 8.15 (s, 1H), 7.27 (t, J=7.8 Hz, 1H), 7.21 (d, J=7.9 Hz, 1H), 7.02 (dd, J=12.3, 5.8 Hz, 1H), 6.78 (d, J=7.7 Hz, 1H), 6.65 (dd, J=11.9, 5.0 Hz, 1H), 6.45 (t, J=6.4 Hz, 1H), 5.18 (d, J=8.5 Hz, 1H), 4.74 (s, 1H), 4.35 (d, J=6.2 Hz, 2H), 3.82 (s, 3H), 3.79 (d, J=11.0 Hz, 1H), 3.69 (s, 1H), 3.62 (s, 1H), 3.04 (t, J=11.1 Hz, 1H), 2.80 (d, J=11.0 Hz, 1H), 2.33-2.67 (m, 1H), 2.19 (s, 3H), 2.12-2.06 (m, 1H), 1.99-1.90 (m, 1H), 1.76-1.68 (m, 1H), 1.62 (d, J=13.5 Hz, 6H).

### Example 131 and 132

### (3-methoxy-4-((3-(3-(2,2,2-trifluoroethyl)-7-(((Z)-1,3,5-trimethylpiperidin-4-yl)amino)benzo[b]thiophen-2-yl) prop-2-yn-1-yl)amino)phenyl)dimethylphosphine oxide(131) and (3-methoxy-4-((3-(3-(2,2,2-trifluoroethyl)-7-(((E)-1,3,5-trimethylpiperidin-4-yl)amino)benzo[b]thiophen-2-yl)prop-2-yn-1-yl)amino)phenyl)dimethylphosphine oxide (132)

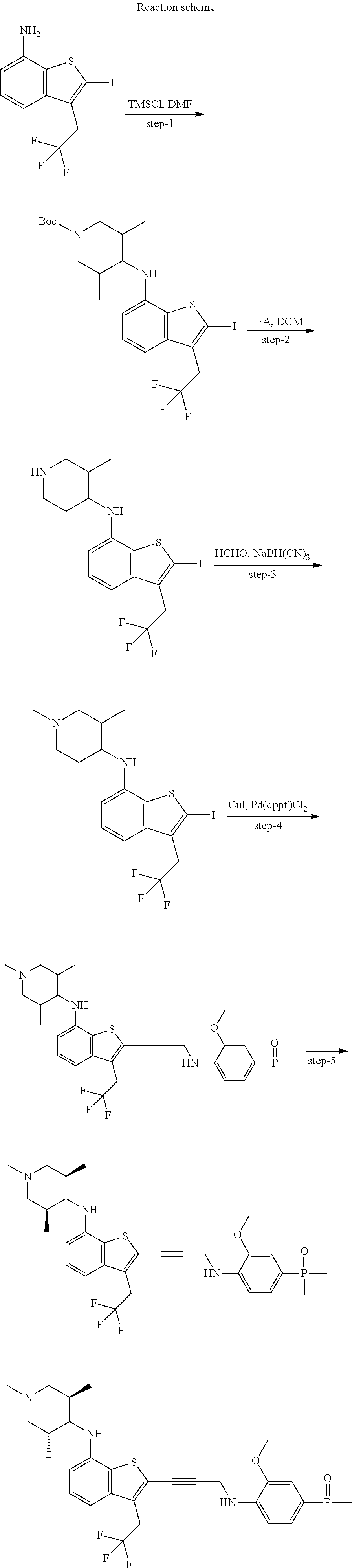

### Experiment Details

### Step 1. tert-butyl 4-((2-iodo-3-(2,2,2-trifluoroethyl)benzo[b]thiophen-7-yl)amino)-3,5-dimethylpiperidine-1-carboxylate

Into a 40-mL sealed tube purged and maintained with an inert atmosphere of nitrogen, was placed 2-iodo-3-(2,2,2-trifluoroethyl)benzo[b]thiophen-7-amine (0.627 g, 1.76 mmol), tert-butyl 3,5-dimethyl-4-oxopiperidine-1-carboxylate (0.741 g, 3.26 mmol), DMF (4 mL). The reaction mixture was stirred at room temperature for 10 mins. Chlorotrimethylsilane was added at 0° C. and then the reaction mixture was stirred at room temperature for 2 h. Tetrahydrofuran solution with borane (1 mol/L, 6 mL) was added to the reaction at 0° C. and stirred for 1 h at room temperature. The mixture was purified with C18 column eluted with ACN/water (v/v=1/1) to afford tert-butyl 4-((2-iodo-3-(2,2,2-trifluoroethyl)benzo[b]thiophen-7-yl)amino)-3,5-dimethylpiperidine-1-carboxylate (0.733 g, 73.44% yield) as little solid. LCMS: m/z=569 [M+1]⁺

### Step 2. N-(2-iodo-3-(2,2,2-trifluoroethyl)benzo[b]thiophen-7-yl)-3,5-dimethylpiperidin-4-amine

Into a 8-mL sealed tube purged and maintained with an inert atmosphere of nitrogen, was placed tert-butyl 4-((2-iodo-3-(2,2,2-trifluoroethyl)benzo[b]thiophen-7-yl)amino)-3,5-dimethylpiperidine-1-carboxylate (0.455 g, 0.80 mmol), DCM (3 mL) and TFA (1 mL). The reaction mixture was stirred at room temperature for 1 h. The mixture was added to sodium carbonate aqueous solution and was stirred for 10 mins. The resulting solution was extracted with DCM (20 mL). The organic layers combined and washed with brine (20 mL), dried over anhydrous Na₂SO₄, the residue was concentrated under vacuum. This resulted in to afford N—(2-iodo-3-(2,2,2-trifluoroethyl)benzo[b]thiophen-7-yl)-3,5-dimethylpiperidin-4-amine (0.334 g, 89.09% yield) as brown solid. LCMS: m/z=469 [M+1]⁺

### Step 3. N-(2-iodo-3-(2,2,2-trifluoroethyl)benzo[b]thiophen-7-yl)-1,3,5-trimethylpiperidin-4-amine

Into a 4-mL sealed tube purged and maintained with an inert atmosphere of nitrogen, was placed N-(2-iodo-3-(2,2,2-trifluoroethyl)benzo[b]thiophen-7-yl)-3,5-dimethylpiperidin-4-amine (0.099 g, 0.21 mmol), Formaldehyde (0.040 g, 0.17 mmol), NaBH(CN)₃ (0.060 g, 1.39 mmol), AcOH (one drop) and EtOH (2 mL). The reaction mixture was stirred at 50° C. for 1 h. The mixture was added to sodium carbonate aqueous solution and was stirred for 10 mins. The resulting solution was extracted with EA (10 mL×2). The organic lay ers combined and washed with brine (20 mL), dried over anhydrous Na₂SO₄, the residue was concentrated under vacuum. This resulted in to afford N-(2-iodo-3-(2,2,2-trifluoroethyl)benzo[b]thiophen-7-yl)-1,3,5-trimethylpiperidin-4-amine (0.117 g, 114.71% yield) as oil. LCMS: m/z=483 [M+1]⁺

### Step 4. (3-methoxy-4-((3-(3-(2,2,2-trifluoroethyl)-7-((1,3,5-trimethylpiperidin-4-yl)amino)benzo[b]thiophen-2-yl)prop-2-yn-1-yl)amino)phenyl)dimethylphosphine oxide

Into a 8-mL sealed tube purged and maintained with an inert atmosphere of nitrogen, was placed N-(2-iodo-3-(2,2,2-trifluoroethyl)benzo[b]thiophen-7-yl)-1,3,5-trimethylpiperidin-4-amine (0.106 g, 0.22 mmol), (3-meth oxy-4-(prop-2-yn-1-ylamino)phenyl)dimethylphosphine oxide (0.119 g, 0.50 mmol), Pd(dppf)Cl₂ (0.015 g, 0.02 mmol), CuI (0.013 g, 0.07 mmol), TEA (0.050 g, 0.49 mmol), DMSO (4 mL). The reaction mixture was stirred at 50° C. for 1 h. The mixture was purified with C18 column eluted with ACN/water (v/v=1/1) to afford (3-methoxy-4-((3-(3-(2,2,2-trifluoroethyl)-7-((1,3,5-trimethylpiperidin-4-yl)amino)benzo[b]thiophen-2-yl)prop-2-yn-1-yl)amino)phenyl)dimethylphosphine oxide (0.150 g, 115.36% yield) as oil. LCMS: m/z=592 [M+1]⁺

### Step 5. (3-methoxy-4-((3-(3-(2,2,2-trifluoroethyl)-7-(((Z)-1,3,5-trimethylpiperidin-4-yl)amino)benzo[b]thiophen-2-yl)prop-2-yn-1-yl)amino)phenyl)dimethylphosphine oxide(131) and (3-methoxy-4-((3-(3-(2,2,2-trifluoro ethyl)-7-(((E)-1,3,5-trimethylpiperidin-4-yl)amino)benzo[b]thiophen-2-yl)prop-2-yn-1-yl)amino)phenyl)dimethylphosphine oxide (132)

(3-methoxy-4-((3-(3-(2,2,2-trifluoroethyl)-7-((1,3,5-trimethylpiperidin-4-yl)amino)benzo[b]thiophen-2-yl)prop-2-yn-1-yl)amino)phenyl)dimethylphosphine oxide (racemic) was purified by preparative HPLC (Mobile Phase A: water (10 mmoL/L ammonium hydroxide), Mobile Phase B: MeOH; Flow rate: 70 mL/min; Gradient: 50-85-95% B (2-30-45 min) 269 inn; RT: 39.83-41.45 min; 42.31-44.16 min) to afford (3-methoxy-4-((3-(3-(2,2,2-trifluoroethyl)-7-(((Z)-1,3,5-trimethylpiperidin-4-yl)amino)benzo[b]thiophen-2-yl)prop-2-yn-1-yl)amino)phenyl)dimethylphosphine oxide (131) (RT: 39.83-41.45 min) (0.020 g, 13.33% yield) as white solid. LCMS: m/z=592 [M+1]⁺

¹H NMR (400 MHz, DMSO) δ 7.17 (m, J=23.6, 13.4, 7.9 Hz, 4H), 6.92-6.79 (m, 1H), 6.64 (d, J=7.8 Hz, 1H), 6.05 (t, J=6.2 Hz, 1H), 5.20 (d, J=8.4 Hz, 1H), 4.34 (d, J=6.3 Hz, 2H), 3.85 (s, 3H), 3.78 (dd, J=21.9, 11.1 Hz, 2H), 3.16 (s, 1H), 2.72 (t, J=15.9 Hz, 1H), 2.62-2.53 (m, 1H), 2.11 (d, J=7.1 Hz, 6H), 1.61 (dd, J=21.9, 12.5 Hz, 6H), 1.23 (s, 1H), 0.90 (dd, J=13.0, 6.6 Hz, 6H).

And (3-methoxy-4-((3-(3-(2,2,2-trifluoroethyl)-7-(((E)-1,3,5-trimethylpiperidin-4-yl)amino)benzo[b]thiophen-2-yl) prop-2-yn-1-yl)amino)phenyl)dimethylphosphine oxide (132) (RT: 42.31-44.16 min) (0.019 g, 12.66% yield) as white solid. LCMS: m/z=592 [M+1]⁺

¹H NMR (400 MHz, DMSO) δ 7.21 (m, J=25.7, 9.3 Hz, 4H), 6.85 (t, J=7.0 Hz, 2H), 6.06 (d, J=6.0 Hz, 1H), 4.34 (d, J=6.1 Hz, 2H), 4.03 (d, J=9.9 Hz, 1H), 3.88-3.72 (m, 5H), 2.44 (d, J=9.4 Hz, 2H), 2.20 (s, 2H), 1.94 (dd, J=28.7, 17.9 Hz, 4H), 1.58 (d, J=13.2 Hz, 6H), 1.24 (s, 2H), 0.79 (t, J=19.5 Hz, 6H).

### Example 135 and 136

### (4-((3-(7-(((3S,4R)-1-ethyl-3-fluoropiperidin-4-yl)amino)-3-(2,2,2-trifluoroethyl)benzo[b]thiophen-2-yl)prop-2-yn-1-yl)amino)-3-methoxyphenyl)dimethylphosphine oxide (135) and (4-((3-(7-(((3R,4S)-1-ethyl-3-fluoropiperidin-4-yl)amino)-3-(2,2,2-trifluoroethyl)benzo[b]thiophen-2-yl)prop-2-yn-1-yl)amino)-3-methoxyphenyl)dimethylphosphine oxide (136)

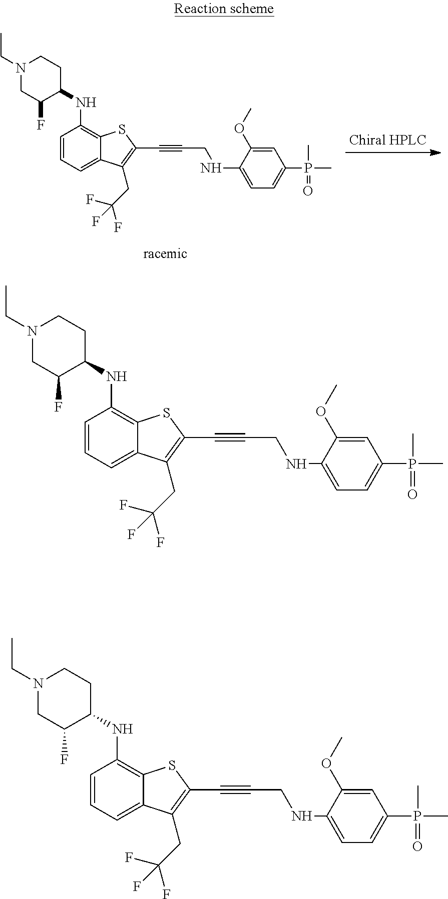 [SMILES: CCN1CC[C@@H](Nc2cccc3c(CC(F)(F)F)c(C#CCNc4ccc(P(C)(C)=O)cc4OC)sc23)[C@@H](F)C1 ; CCN1CC[C@@H](Nc2cccc3c(CC(F)(F)F)c(C#CCNc4ccc(P(C)(C)=O)cc4OC)sc23)[C@@H](F)C1 ; CCN1CC[C@H](Nc2cccc3c(CC(F)(F)F)c(C#CCNc4ccc(P(C)(C)=O)cc4OC)sc23)[C@H](F)C1]

### Experimental Details

### Step 1. (4-((3-(7-(((3S,4R)-1-ethyl-3-fluoropiperidin-4-yl)amino)-3-(2,2,2-trifluoroethyl)benzo[b]thiophen-2-yl)prop-2-yn-1-yl)amino)-3-methoxyphenyl)dimethylphosphine oxide (135) and (4-((3-(7-(((3R,4S)-1-ethyl-3-fluoropiperidin-4-yl)amino)-3-(2,2,2-trifluoroethyl)benzo[b]thiophen-2-yl)prop-2-yn-1-yl)amino)-3-methoxyphenyl)dimethylphosphine oxide (136)

(4-((3-(7-(((Z)-1-ethyl-3-fluoropiperidin-4-yl)amino)-3-(2,2,2-trifluoroethyl)benzo[b]thiophen-2-yl)prop-2-yn-1-yl) amino)-3-methoxyphenyl)dimethylphosphine oxide(racemic) (0.189 g, 317.31 μmol) was purified by Chiral H PLC (Column: SC, Mobile Phase A: HeX (0.2% IPA.M), Mobile Phase B: EtOH; Flow rate: 20 mL/min; A/B (V/V=60/40); 220 min; RT: 12.821 min) to provide 0.057 g (30.16% yield) of (4-((3-(7-(((3S,4R)-1-et hyl-3-fluoropiperidin-4-yl)amino)-3-(2,2,2-trifluoroethyl)benzo[b]thiophen-2-yl)prop-2-yn-1-yl)amino)-3-methoxyphenyl)dimethylphosphine oxide (135) as white solid. LCMS: m/z=596 [M+1]⁺.

¹H NMR (400 MHz, DMSO) δ 7.30-7.23 (m, 1H), 7.20 (d, J=8.3 Hz, 2H), 7.14 (d, J=11.2 Hz, 1H), 6.87-6.82 (m, 1H), 6.78 (d, J=7.6 Hz, 1H), 6.06 (t, J=6.3 Hz, 1H), 5.14 (d, J=8.4 Hz, 1H), 4.89-4.73 (m, 1H), 4.34 (d, J=6.4 Hz, 2H), 3.85 (s, 3H), 3.82-3.76 (m, 2H), 3.15-3.07 (m, 1H), 2.89 (d, J=9.4 Hz, 1H), 2.40-2.32 (m, 2H), 2.28 (d, J=12.6 Hz, 1H), 2.18 (d, J=12.7 Hz, 1H), 2.07 (t, J=11.1 Hz, 1H), 1.94-1.88 (m, 1H), 1.75 (s, 1H), 1.59 (s, 3H), 1.56 (s, 3H), 0.99 (t, J=7.1 Hz, 3H).

(4-((3-(7-(((Z)-1-ethyl-3-fluoropiperidin-4-yl)amino)-3-(2,2,2-trifluoroethyl)benzo[b]thiophen-2-yl)prop-2-yn-1-yl) amino)-3-methoxyphenyl)dimethylphosphine oxide(racemic) (0.189 g, 317.31 μmol) was purified by Chiral H PLC (Column: SC, Mobile Phase A: HeX (0.2% IPA.M), Mobile Phase B: EtOH; Flow rate: 20 mL/min; A/B (V/V=60/40); 220 nm; RT: 14.348 min) to provide 0.064 g (33.86% yield) of (4-((3-(7-(((3R,4S)-1-et hyl-3-fluoropiperidin-4-yl)amino)-3-(2,2,2-trifluoroethyl)benzo[b]thiophen-2-yl)prop-2-yn-1-yl)amino)-3i-methoxyphenyl)dimethylphosphine oxide (136) as white solid. LCMS: m/z=596 [M+1]⁺.

¹H NMR (400 MHz, DMSO) δ 7.29-7.23 (m, 1H), 7.20 (d, J=8.1 Hz, 2H), 7.16-7.11 (m, 1H), 6.87-6.82 (m, 1H), 6.78 (d, J=7.7 Hz, 1H), 6.06 (t, J=6.3 Hz, 1H), 5.14 (d, J=8.5 Hz, 1H), 4.88-4.72 (m, 1H), 4.34 (d, J=6.3 Hz, 2H), 3.85 (s, 3H), 3.82-3.74 (m, 2H), 3.70 (s, 2H), 3.15-3.07 (m, 1H), 2.89 (d, J=12.3 Hz, 1H), 2.40-2.32 (m, 2H), 2.31-2.25 (m, 1H), 2.22-2.15 (m, 1H), 2.11-2.04 (m, 1H), 1.96-1.87 (m, 1H), 1.75 (s, 1H), 1.59 (s, 3H), 1.56 (s, 3H), 1.27-1.22 (m, 1H).

### Example 137 and 138

### (4-((3-(7-(((3R,4S)-3-fluoro-1-methylpiperidin-4-yl)amino)-3-(2,2,2-trifluoroethyl)benzo[b]thiophen-2-yl)prop-2-yn-1-yl)amino)-3-(trifluoromethoxy)phenyl)dimethylphosphine oxide (137) and (4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-(trifluoromethoxy)phenyl)dimethylphosphine oxide (138)

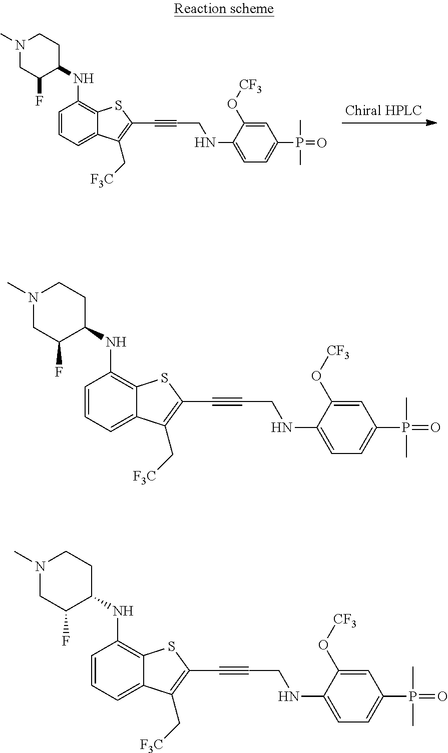 [SMILES: CN1CC[C@@H](Nc2cccc3c(CC(F)(F)F)c(C#CCNc4ccc(P(C)(C)=O)cc4OC(F)(F)F)sc23)[C@@H](F)C1 ; CN1CC[C@@H](Nc2cccc3c(CC(F)(F)F)c(C#CCNc4ccc(P(C)(C)=O)cc4OC(F)(F)F)sc23)[C@@H](F)C1 ; CN1CC[C@H](Nc2cccc3c(CC(F)(F)F)c(C#CCNc4ccc(P(C)(C)=O)cc4OC(F)(F)F)sc23)[C@H](F)C1]

### Experimental Details

### Step 1. (4-((3-(7-(((3R,4S)-3-fluoro-1-methylpiperidin-4-yl)amino)-3-(2,2,2-trifluoroethyl)benzo[b]thiophen-2-yl)prop-2-yn-1-yl)amino)-3-(trifluoromethoxy)phenyl)dimethylphosphine oxide (137) and (4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-(trifluoromethoxy)phenyl)dimethylphosphine oxide (138)

(4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-(trifluoromethoxy)phenyl)dimethylphosphine oxide(racemic) (0.080 g, 125.87 μmol) was purified by Chiral HPLC (Column: IG, Mobile Phase A: (HeX:DCM=3:1)(0.2% IPA.M), Mobile Phase B: EtOH; Flow rate: 20 mL/min; A/B (V/V=80/20); 220 nm; RT: 6.029 min) to provide 0.032 g (43.00% yield) of (4-((3-(7-(((3R,4S)-3-fluoro-1-methylpiperidin-4-yl)amino)-3-(2,2,2-trifluoroethyl)benzo[b]thiophen-2-yl)prop-2-yn-1-yl)amino)-3-(trifluoromethoxy)phenyl)dimethylphosphine oxide as white solid. LCMS: m/z=636 [M+1]⁺.

¹H NMR (400 MHz, DMSO) δ 7.57 (s, 2H), 7.33-7.18 (m, 2H), 7.11 (s, 1H), 6.90-6.76 (m, 2H), 5. 17 (s, 1H), 4.90-4.71 (m, 1H), 4.40 (s, 1H), 3.79 (s, 2H), 3.05 (s, 1H), 2.80 (s, 2H), 2.19 (d, J=8.1 Hz, 3H), 2.11-1.87 (m, 3H), 1.72 (s, 1H), 1.65-1.54 (m, 5H), 1.25 (s, 2H).

(4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-(trifluoromethoxy)phenyl)dimethylphosphine oxide(racemic) (0.080 g, 125.87 μmol) was purified by Chiral HPLC (Column: IG, Mobile Phase A: (HeX:DCM=3:1)(0.2% IPA.M), Mobile Phase B: EtOH; Flow rate: 20 mL/min; A/B (V/V=80/20); 220 nm; RT: 6.926 min) to provide 0.035 g (43.75% yield) of (4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-(trifluoromethoxy)phenyl)dimethylphosphine oxide as white solid. LCMS: m/z=636 [M+1]⁺.

¹H NMR (400 MHz, DMSO) δ 7.62-7.50 (m, 2H), 7.27 (t, J=7.6 Hz, 1H), 7.20 (d, J=7.7 Hz, 1H), 7.10 (d, J=8.2 Hz, 1H), 6.87 (s, 1H), 6.78 (d, J=7.6 Hz, 1H), 5.23 (d, J=8.4 Hz, 1H), 4.89-4.71 (m, 1H), 4.40 (d, J=5.5 Hz, 2H), 3.85-3.74 (m, 2H), 3.71-3.56 (m, 1H), 3.03 (t, J=10.3 Hz, 1H), 2.79 (d, J=10.9 Hz, 1H), 2.32-2.23 (m, 1H), 2.16 (s, 3H), 2.10-2.03 (m, 1H), 2.00-1.87 (m, 1H), 1.71 (d, J=12.1 Hz, 1H), 1.60 (d, J=13.3 Hz, 6H).

### Example 139

### (4-((3-(7-(((E)-3-fluoro-1-methylpiperidin-4-yl)amino)-3-(4-fluorophenyl)benzo[b]thiophen-2-yl)prop-2-yn-1-yl)amino)-3-methoxyphenyl)dimethylphosphine oxide (139)

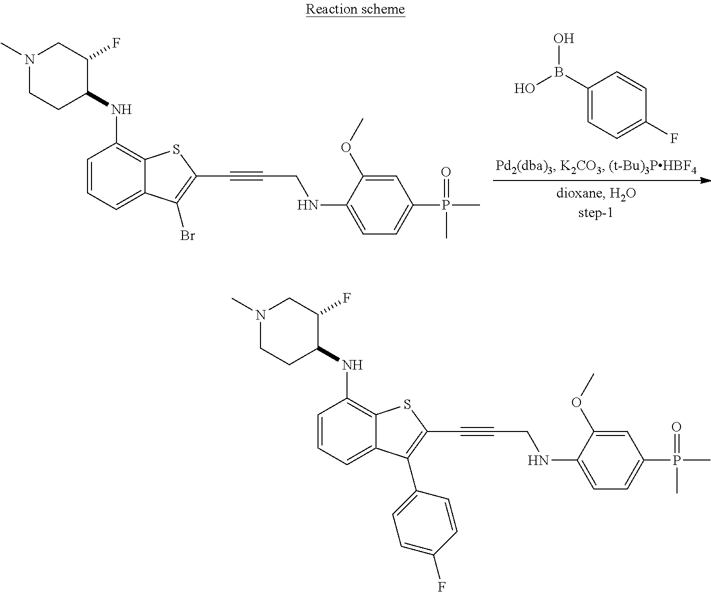

### Experimental Details

### Step 1. (4-((3-(7-(((E)-3-fluoro-1-methylpiperidin-4-yl)amino)-3-(4-fluorophenyl)benzo[b]thiophen-2-yl)prop-2-yn-1-yl)amino)-3-methoxyphenyl)dimethylphosphine oxide (139)

Into a 25-mL three necked bottle and maintained with an inert atmosphere of nitrogen, was placed (4-((3-(3-bromo-7-(((E)-3-fluoro-1-methylpiperidin-4-yl)amino)benzo[b]thiophen-2-yl)prop-2-yn-1-yl)amino)-3-methoxyphenyl)dimethylphosphine oxide (30.84 mg, 53.31 umol), (4-fluorophenyl)boronic acid (11.35 mg, 81.11 umol), Pd2(dba)3 (11.08 mg, 12.09 umol), (t-Bu)₃PHBF·₄ (3.49 mg, 12.02 umol), Cs₂CO₃ (51.45 mg, 157.90 umol), dioxane (0.4 mL) and H₂O (0.08 mL). The reaction mixture was stirred at 80° C. for 3 h. The reaction mixture was filtered through celite pad and celite pad was washed with ACN (3×20 mL). The filterate was then concentrated under reduced pressure to afford the crude product. The crude product was purified by preparative HPLC (Mobile Phase A: water (0.1% ammonium hydroxide), Mobile Phase B: ACN; Flow rate: 40 mL/min; Gradient: 30-60-60% B (2-30-60 min); 262 nm; RT: 28.022-29.057) to provide the desired product. This resulted in 9.42 mg (29.76% yield) (4-((3-(7-(((E)-3-fluoro-1-methylpiperidin-4-yl)amino)-3-(4-fluorophenyl)benzo[b]thiophen-2-yl)prop-2-yn-1-yl)amino)-3-methoxyphenyl)dimethylphosphine oxide (139) as white solid. LCMS: m/z=594 [M+1]⁺.

¹H NMR (400 MHz, DMSO-d₆) δ 7.55-7.46 (m, 2H), 7.28-7.11 (m, 5H), 6.96 (d, J=8.0 Hz, 1H), 6.80 (d, J=7.9 Hz, 1H), 6.71-6.67 (m, 1H), 6.00 (t, J=6.3 Hz, 1H), 5.16 (d, J=8.6 Hz, 1H), 4.81 (d, J=49.6 Hz, 1H), 4.21 (d, J=6.3 Hz, 2H), 3.83 (s, 3H), 3.74-3.62 (m, 1H), 3.04 (t, J=11.5 Hz, 1H), 2.85-2.78 (m, 1H), 2.28 (d, J=13.0 Hz, 1H), 2.19 (s, 3H), 2.09 (t, J=11.5 Hz, 1H), 2.01-1.92 (m, 1H), 1.73 (d, J=11.9 Hz, 1H), 1.59 (d, J=13.1 Hz, 6H).

### Example 140 and 141

### dicyclopropyl(4-((3-(7-(((3R,4S)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)phosphine oxide (140) and dicyclopropyl(4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)phosphine oxide (141)

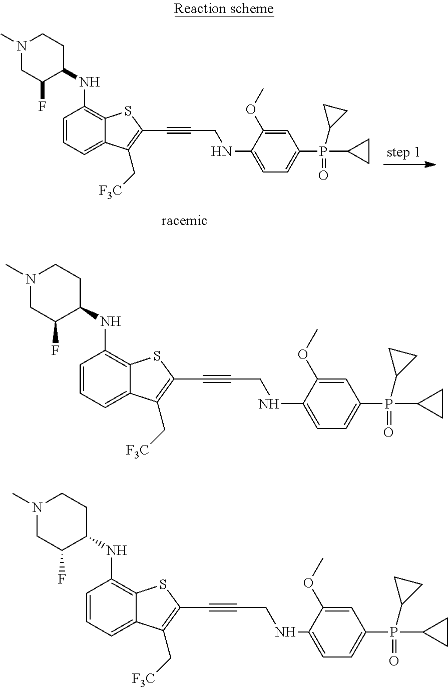 [SMILES: COc1cc(P(=O)(C2CC2)C2CC2)ccc1NCC#Cc1sc2c(N[C@@H]3CCN(C)C[C@@H]3F)cccc2c1CC(F)(F)F ; COc1cc(P(=O)(C2CC2)C2CC2)ccc1NCC#Cc1sc2c(N[C@@H]3CCN(C)C[C@@H]3F)cccc2c1CC(F)(F)F ; COc1cc(P(=O)(C2CC2)C2CC2)ccc1NCC#Cc1sc2c(N[C@H]3CCN(C)C[C@H]3F)cccc2c1CC(F)(F)F]

### Experimental Details

### Step 1. dicyclopropyl(4-((3-(7-(((3R,4S)-3-fluoro-1-methylpiperidin-4-yl)amino)-3-(2,2,2-trifluoroethyl)benzo [b]thiophen-2-yl)prop-2-yn-1-yl)amino)-3-methoxyphenyl)phosphine oxide (140) and dicyclopropyl(4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)phosphine oxide (141)

The racemic sample dicyclopropyl(4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)phosphine oxide (150 mg, 0.23 mmol) was separated by pre-CHIRAL-HPLC (Column:IG, Mobile Phase A: (HeX:DCM=3:1)(0.2% IPA.M), Mobile Phase B; A:B (V:V=70:30), Flow rate: 20 mL/min; RT: 7.596 min) to provide 69.00 mg (46.00% yield) of dicyclopropyl(4-((3-(7-(((3R,4S)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)phosphine oxide (140) as off white solid. LCMS: m/z=634 [M+1]⁺

¹H NMR (400 MHz, DMSO-d₆) δ 7.32-7.19 (m, 3H), 7.16-7.11 (m, 1H), 6.88-6.83 (m, 1H), 6.78 (d, J=7.6 Hz, 1H), 6.09 (t, J=6.3 Hz, 1H), 5.17 (d, J=8.5 Hz, 1H), 4.80 (d, J=49.7 Hz, 1H), 4.34 (d, J=6.3 Hz, 2H), 3.84 (d, J=8.5 Hz, 3H), 3.81 (d, J=11.1 Hz, 1H), 3.74-3.56 (m, 1H), 3.10-2.91 (m, 1H), 2.80 (d, J=11.2 Hz, 1H), 2.32-2.15 (m, 4H), 2.09 (t, J=11.2 Hz, 1H), 2.01-1.89 (m, 1H), 1.72 (d, J=10.0 Hz, 1H), 1.26-1.11 (m, 3H), 0.81-0.51 (m, 8H).

The racemic sample dicyclopropyl(4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)phosphine oxide (150 mg, 0.23 mmol) was separated by pre-CHIRAL-HPLC (Column:IG, Mobile Phase A: (HeX:DCM=3:1)(0.2% IPA.M), Mobile Phase B; A:B=70:30, Flow rate: 20 mL/min; RT: 9.409 min) to provide 69.00 mg (46.00% yield) of dicyclopropyl(4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)phosphine oxide (141) as white solid. LCMS: m/z=634 [M+1]⁺

¹H NMR (400 MHz, DMSO-d₆) δ 7.35-7.17 (m, 3H), 7.15 (d, J=11.3 Hz, 1H), 6.89-6.82 (m, 1H), 6.78 (d, J=7.7 Hz, 1H), 6.09 (t, J=6.2 Hz, 1H), 5.18 (d, J=8.5 Hz, 1H), 4.80 (d, J=49.8 Hz, 1H), 4.34 (d, J=6.2 Hz, 2H), 3.94-3.75 (m, 5H), 3.65 (d, J=29.0 Hz, 1H), 3.03 (t, J=10.8 Hz, 1H), 2.79 (d, J=10.9 Hz, 1H), 2.30-2.12 (m, 4H), 2.12-2.02 (m, 1H), 1.98-1.91 (m, 1H), 1.71 (d, J=10.4 Hz, 1H), 1.25-1.10 (m, 2H), 0.88-0.51 (m, 8H).

### Example 142

### (4-((3-(7-(((Z)-3-fluoro-1-methylpiperidin-4-yl)amino)-3-(4-fluorophenyl)benzo[b]thiophen-2-yl)prop-2-yn-1-yl)amino)-3-methoxyphenyl)dimethylphosphine oxide (142)

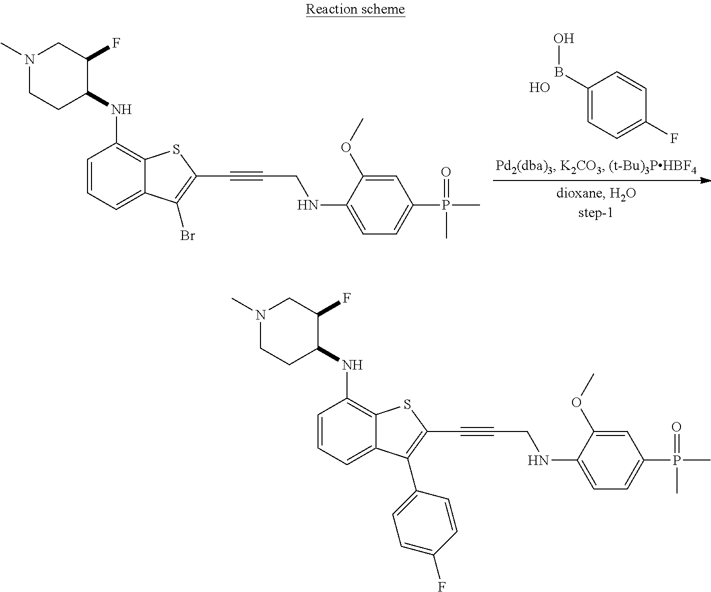

### Experimental Details

### Step 1. (4-((3-(7-(((Z)-3-fluoro-1-methylpiperidin-4-yl)amino)-3-(4-fluorophenyl)benzo[b]thiophen-2-yl)prop-2-yn-1-yl)amino)-3-methoxyphenyl)dimethylphosphine oxide (142)

Into a 25-mL three necked bottle and maintained with an inert atmosphere of nitrogen, was placed (4-((3-(3-bromo-7-(((Z)-3-fluoro-1-methylpiperidin-4-yl)amino)benzo[b]thiophen-2-yl)prop-2-yn-1-yl)amino)-3-methoxyphenyl)dimethylphosphine oxide (28.29 mg, 48.90 umol), (4-fluorophenyl)boronic acid (10.16 mg, 72.61 umol), Pd₂(dba)₃ (8.42 mg, 8.41 umol), (t-Bu)₃PHBF·₄ (2.44 mg, 8.41 umol), Cs₂CO₃ (50.52 mg, 155.05 umol), dioxane (0.4 mL) and H₂O (0.08 mL). The reaction mixture was stirred at 80° C. for 3 h. The reaction mixture was filtered through celite pad and celite pad was washed with ACN (3×20 mL). The filterate was then concentrated under reduced pressure to afford the crude product. The crude product was purified by preparative HPLC (Mobile Phase A: water (0.1% ammonium hydroxide), Mobile Phase B: ACN; Flow rate: 40 mL/min; Gradient: 25-60-85% B (2-30-60 min); 262 nm; RT: 31.932-32.737) to provide the desired product. T his resulted in 5.07 mg (17.46% yield) (4-((3-(7-(((Z)-3-fluoro-1-methylpiperidin-4-yl)amino)-3-(4-fluorophenyl)benzo[b]thiophen-2-yl)prop-2-yn-1-yl)amino)-3-methoxyphenyl)dimethylphosphine oxide (142) as white solid. LCMS: m/z=594 [M+1]⁺.

¹H NMR (400 MHz, DMSO-d₆) δ 7.54-7.45 (m, 2H), 7.27-7.06 (m, 5H), 6.90 (d, J=7.9 Hz, 1H), 6.76 (d, J=7.9 Hz, 1H), 6.72-6.67 (m, 1H), 6.00 (t, J=6.3 Hz, 1H), 5.59 (d, J=8.6 Hz, 1H), 4.73-4.56 (m, 1H), 4.21 (d, J=6.3 Hz, 2H), 3.83 (s, 3H), 3.61 (d, J=4.6 Hz, 1H), 3.21-3.15 (m, 1H), 2.70 (d, J=11.4 Hz, 1H), 2.24 (s, 3H), 2.14-1.89 (m, 3H), 1.60 (d, J=13.2 Hz, 6H).

### Example 143

### (3-methoxy-4-((3-(3-(2,2,2-trifluoroethyl)-7-((1,2,6-trimethylpiperidin-4-yl)amino)benzo[b]thiophen-2-yl)prop-2-yn-1-yl)amino)phenyl)dimethylphosphine oxide (143)

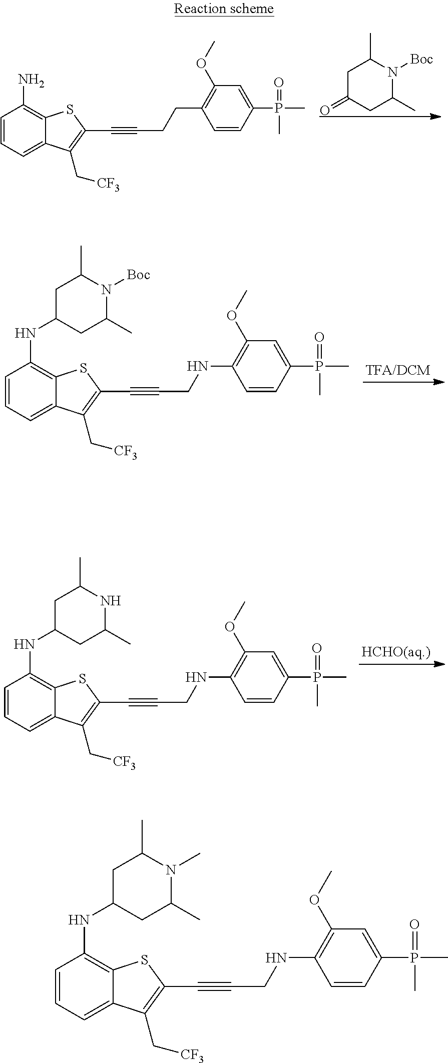

### Experiment Details

### Step 1. tert-butyl 8-((2-(3-((4-(dimethylphosphoryl)-2-methoxyphenyl)amino)prop-1-yn-1-yl)-3-(2,2,2-trifluoroethyl)benzo[b]thiophen-7-yl)amino)-3-azabicyclo[3.2.1]octane-3-carboxylate

Into a 25 mL flask was placed (4-((3-(7-amino-3-(2,2,2-trifluoroethyl)benzo[b]thiophen-2-yl)prop-2-yn-1-yl)amino)-3-methoxyphenyl)dimethylphosphine oxide (0.198 g, 424.48 μmol), tert-butyl 2,6-dimethyl-4-oxopiperidine-1-carboxylate (0.163 g, 717.12 μmol), PhSiH3 (0.116 g, 1.07 mmol), Dibutyltin dichloride (0.555 g, 1.83 mmol), THF (3 mL). The reaction was stirred overnight at room temperature. LCMS monitored the reaction was complete. The reaction was quenched by MeOH, purified with C₁₈ column (ACN/water v/v=1/1) to afford tert-butyl 8-((2-(3-((4-(dimethylphosphoryl)-2-methoxyphenyl)amino)prop-1-yn-1-yl)-3-(2,2,2-trifluoroethyl)benzo[b]thiophen-7-yl)amino)-3-azabicyclo[3.2.1]octane-3-carboxylate (0.429 g, 632.97 μmol, 149.12% yield) as light brown solid. LCMS: m/z=678[M+1]⁺.

### Step 2. (4-((3-(7-((2,6-dimethylpiperidin-4-yl)amino)-3-(2,2,2-trifluoroethyl)benzo[b]thiophen-2-yl)prop-2-yn-1-yl)amino)-3-methoxyphenyl)dimethylphosphine oxide

Into a 4 mL flask was placed tert-butyl 8-((2-(3-((4-(dimethylphosphoryl)-2-methoxyphenyl)amino)prop-1-yn-1-yl)-3-(2,2,2-trifluoroethyl)benzo[b]thiophen-7-yl)amino)-3-azabicyclo[3.2.1]octane-3-carboxylate (0.363 g, 535.59 μmol), TFA (0.5 mL), DCM (2.5 mL). The reaction was stirred for 0.5 h at room temperature. LCMS showed the reaction was complete. The reaction quenched with saturated NaHCO₃ aq., The solution was diluted with water (2 mL), extracted with EA (2 mL×2). The combined organic layers were washed with water (2 mL) and brine (2 mL) successively, separated, then concentrated with vacuum to afford crude (4-((3-(7-((2,6-dimethylpiperidin-4-yl)amino)-3-(2,2,2-trifluoroethyl)benzo[b]thiophen-2-yl)prop-2-yn-1-yl)amino)-3-methoxyphenyl)dimethylphosphine oxide (0.275 g, 476.08 μmol, 88.89% yield) as an off-white solid. LCMS: m/z=578[M+1]⁺

### Step 3. (3-methoxy-4-((3-(3-(2,2,2-trifluoroethyl)-7-((1,2,6-trimethylpiperidin-4-yl)amino)benzo[b]thiophen-2-yl)prop-2-yn-1-yl)amino)phenyl)dimethylphosphine oxide (143)

Into a 4 mL vail was placed (4-((3-(7-((2,6-dimethylpiperidin-4-yl)amino)-3-(2,2,2-trifluoroethyl)benzo[b]thiophen-2-yl)prop-2-yn-1-yl)amino)-3-methoxyphenyl)dimethylphosphine oxide (0.192 g, 332.39 μmol), formaldehyde (0.038 g, 442.95 μmol), sodium cyanoboronhydride (0.099 g, 2.31 mmol), MeOH (1 mL), acetic acid (0.01 mL). The reaction was stirred for 1.5 h at room temperature. LCMS showed the reaction was complete. The residue was purified with prep-HPLC (Mobile Phase A: water (formic acid), Mobile Phase B: AC N; Flow rate: 70 mL/min; Gradient: 25-45-45% B (2-30-60 min); 262 nm; RT: 27.501-29.585 min) to afford (3-methoxy-4-((3-(3-(2,2,2-trifluoroethyl)-7-((1,2,6-trimethylpiperidin-4-yl)amino)benzo[b]thiophen-2-yl)prop-2-yn-1-yl)amino)phenyl)dimethylphosphine oxide (143) (0.030 g, 50.70 μmol, 11.40% yield) as a white solid. LCMS: m/z=592[M−1]⁺

¹H NMR (600 MHz, DMSO-d₆) δ 7.31-7.19 (m, 2H), 7.17-7.10 (m, 2H), 6.85 (dd, J=8.1, 2.9 Hz, 1H), 6.67 (d, J=7.8 Hz, 1H), 6.06 (t, J=6.4 Hz, 1H), 5.27 (d, J=8.2 Hz, 1H), 4.34 (d, J=6.4 Hz, 2H), 3.86 (s, 3H), 3.81-3.75 (m, 2H), 3.53-3.42 (m, 1H), 2.15 (s, 5H), 1.87 (dd, J=12.3, 3.6 Hz, 2H), 1.59 (d, J=13.1 Hz, 6H), 1.31-1.25 (m, 2H), 1.06 (d, J=6.1 Hz, 6H).

### Example 144 and 158

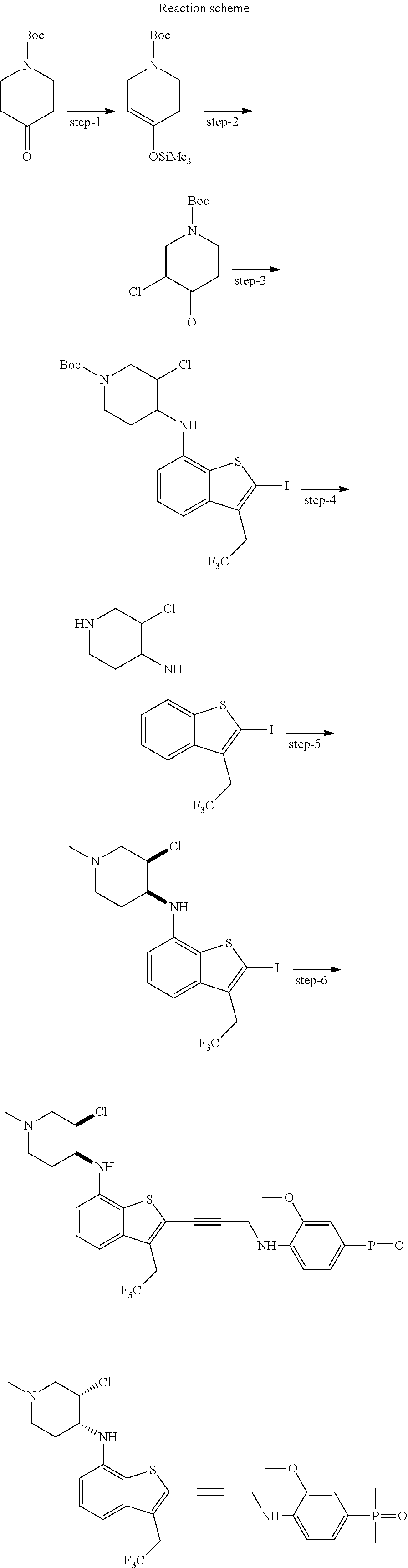

### Experiment Details

### Step 1. Synthesis of tert-butyl 4-trimethylsilyloxy-3,6-dihydro-2H-pyridine-1-carboxylate

Into a 500-mL round-bottom-flask was placed tert-butyl 4-oxopiperidine-1-carboxylate (20.09 g, 100.83 mmol), toluene (250 mL), triethylamine (30.63 g, 302.70 mmol), chlorotrimethylsilane (19.29 g, 177.55 mmol). The mixture was stirred at 100° C. under nitrogen atmosphere for 3d. The resulted reaction was concentrated to give crude product. Then the crude was purified by silica gel chromatography column eluted with n-Hept/DCM (v/v=1/2). This resulted in 16.84 g (30.77% yield) of tert-butyl 4-trimethylsilyloxy-3,6-dihydro-2H-pyridine-1-carboxylate. LCMS: m/z=272 [M+1]⁺.

### Step 2. Synthesis of tert-butyl 3-chloro-4-oxo-piperidine-1-carboxylate

Into a 250-mL round-bottom-flask was placed tert-butyl 4-trimethylsilyloxy-3,6-dihydro-2H-pyridine-1-carboxylate (7.02 g, 25.86 mmol), acetone (60 mL), water (15 mL). Then 1-Chloro-2,5-piperidinedione (6.87 g, 51.45 mmol) was added slowly after the mixture was cooled to 0° C. Then the mixture was allowed to warm to 25° C. and stirred at this temperature overnight. The resulted reaction was concentrated to give crude product. The crude was purified by C₁₈ chromatography column eluted with ACN/water (v/v=1/2). This resulted in 3.00 g (49.64% yield) of tert-butyl 3-chloro-4-oxo-piperidine-1-carboxylate. LCMS: m/z=234 [M+1]⁺.

### Step 3. Synthesis of tert-butyl 3-chloro-4-[[2-iodo-3-(2,2,2-trifluoroethyl)benzothiophen-7-yl]amino]piperidine-1-carboxylate

Into a 100-mL round-bottom-flask was placed tert-butyl 3-chloro-4-oxo-piperidine-1-carboxylate (2.94 g, 12.58 mmol), 2-iodo-3-(2,2,2-trifluoroethyl)benzo[b]thiophen-7-amine (2.95 g, 8.26 mmol), N,N-Dimethylformamide (30 mL). Then chlorotrimethylsilane (9.00 g, 82.84 mmol) was added slowly after the mixture was coo led to 0° C. Ten the mixture was allowed to warm to 25° C. and stirred at this temperature for 2 h. Then borane-tetrahydrofuran complex (1M, 16 mL) was added dropwise after the mixture was cooled to 0° C. and stirred at this temperature for further 1 h. The resulted reaction was quenched with MeOH (16 mL) and concentrated. The residue was diluted with water (100 mL) and extracted with EA (100 mL×3). The organic phase was dried with Na₂SO₄ and concentrated. This resulted in 5.89 g (crude) of tert-butyl 3-chloro-4-[[2-iodo-3-(2,2,2-trifluoroethyl)benzothiophen-7-yl]amino]piperidine-1-carboxylate. LCMS: m/z=575 [M+1]⁺

### Step 4. Synthesis of 3-chloro-N-[2-iodo-3-(2,2,2-trifluoroethyl)benzothiophen-7-yl]piperidin-4-amine

Into a 250-mL round-bottom-flask was placed tert-butyl 3-chloro-4-[[2-iodo-3-(2,2,2-trifluoroethyl)benzothiophen-7-yl]amino]piperidine-1-carboxylate (4.13 g, 7.19 mmol), dichloromethane (40 mL), trifluoroacetic acid (10 mL). Then the mixture was stirred at 25° C. for 2 h. The resulted reaction was adjusted to ph˜8 with NaHCO₃ (aq) and extracted with EA (100 mL×3). The organic phase was dried with Na₂SO₄ and concentrated. This resulted in 4.92 g (crude) of 3-chloro-N-[2-iodo-3-(2,2,2-trifluoroethyl)benzothiophen-7-yl]piperidin-4-amine. LCMS: m/z=475 [M+1]⁺.

### Step 5. Synthesis of (Z)-3-chloro-N-[2-iodo-3-(2,2,2-trifluoroethyl)benzothiophen-7-yl]-1-methyl-piperidin-4-amine

Into a 250-mL round-bottom-flask was placed (Z)-3-chloro-N-[2-iodo-3-(2,2,2-trifluoroethyl)benzothiophen-7-yl]piperidin-4-amine (4.80 g, 10.11 mmol), ethanol (50 mL), sodium cyanoboronhydride (1.83 g, 42.68 mmol), formaldehyde (1.06 g, 33% in water). The mixture was stirred at 50° C. for 2 h. The resulted reaction w as diluted with water (200 mL) and extracted with EA (100 mL×3). The organic phase was dried with Na₂SO₄ and concentrated. This resulted in 4.25 g (crude) of (Z)-3-chloro-N-[2-iodo-3-(2,2,2-trifluoroethyl)benzothiophen-7-yl]-1-methyl-piperidin-4-amine. LCMS: m/z=489 [M+1]⁺.

### Step 6. Synthesis of (3R,4S)-3-chloro-N-[2-[3-(4-dimethylphosphoryl-2-methoxy-anilino)prop-1-ynyl]-3-(2,2,2-trifluoroethyl)benzothiophen-7-yl]-1-methyl-piperidin-4-amine(144) and (3S,4R)-3-chloro-N-[2-[3-(4-dimethylphosphoryl-2-methoxy-anilino)prop-1-ynyl]-3-(2,2,2-trifluoroethyl)benzothiophen-7-yl]-1-methyl-piperidin-4-amine (158)

Into a 40-mL vial was placed (Z)-3-chloro-N-[2-iodo-3-(2,2,2-trifluoroethyl)benzothiophen-7-yl]-1-methyl-piperidin-4-amine (3.258 g, 6.67 mmol), (3-methoxy-4-(prop-2-yn-1-ylamino)phenyl)dimethylphosphine oxide (1.841 g, 7.76 mmol), bis(triphenylphosphine)palladium(II) chloride (0.475 g, 672.87 μmol), copper(I) iodide (0.185 g, 973.68 μmol), N,N-Diisopropylethylamine (3.681 g, 28.48 mmol), methyl sulfoxide (30 mL). The mixture was stirred at 50° C. for 3 h. The resulted reaction was purified by C₁₈ chromatography column eluted with ACN/water (0.1% ammonium bicarbonate) (v/v=1/2) to give the racemic product. The racemic product was separated by prep-chiral HPLC (Column:SC, Mobile Phase A: HeX (0.2% IPA.M), Mobile Phase B: EtOH; Flow rate: 20 mL/min; A:B (v:v=60:40); 220 nm; RT: 5.891 min) to provide This resulted in 0.149 g (37.25% yield) of (3R,4S)-3-chloro-N-[2-[3-(4-dimethylphosphoryl-2-methoxy-anilino)prop-1-ynyl]-3-(2,2,2-trifluoroethyl)benzothiophen-7-yl]-1-methyl-piperidin-4-amine(144) as white solid. LCMS: m/z=598 [M+1]⁺.

¹H NMR (400 MHz, DMSO-d6) δ 7.24 (dq, J=15.9, 7.9 Hz, 3H), 7.14 (d, J=11.8 Hz, 1H), 6.88-6.77 (m, 2H), 6.07 (t, J=6.3 Hz, 1H), 5.00 (d, J=8.3 Hz, 1H), 4.59 (s, 1H), 4.34 (d, J=6.3 Hz, 2H), 3.88-3.83 (m, 3H), 3.80 (d, J=11.3 Hz, 3H), 2.94 (d, J=12.8 Hz, 1H), 2.80 (d, J=10.7 Hz, 1H), 2.47 (s, 1H), 2.19 (s, 3H), 2.09 (t, J=11.4 Hz, 1H), 2.04-1.91 (m, 1H), 1.67 (d, J=12.6 Hz, 1H), 1.58 (dd, J=13.2, 1.4 Hz, 6H).

The racemic product was separated by prep-chiral HPLC (Column:SC, Mobile Phase A: HeX (0.2% IPA.M), Mobile Phase B: EtOH; Flow rate: 20 mL/min; A:B (v:v=60:40); 220 nm; RT: 7.875 min) to provide 0.114 g (28.00% yield) of (3S,4R)-3-chloro-N-[2-[3-(4-dimethylphosphoryl-2-methoxy-anilino)prop-1-ynyl]-3-(2,2,2-trifluoroethyl)benzothiophen-7-yl]-1-methyl-piperidin-4-amine (158) as white solid. LCMS: m/z=598 [M+1]⁺.

¹H NMR (400 MHz, DMSO-d6) δ 7.25 (t, J=7.1 Hz, 1H), 7.21 (t, J=7.5 Hz, 2H), 7.12 (dd, J=11.7, 5.7 Hz, 1H), 6.81 (d, J=14.3, 6.8 Hz, 2H), 6.04 (d, J=6.6 Hz, 1H), 5.10-4.81 (m, OH), 4.57 (s, 1H), 4.32 (d, J=6.3 Hz, 2H), 3.83 (d, J=5.9 Hz, 4H), 3.79 (s, 2H), 2.92 (s, 1H), 2.78 (s, 1H), 2.50 (d, J=2.0 Hz, 1H), 2.18 (d, J=5.9 Hz, 3H), 2.08 (s, 1H), 1.97 (s, 1H), 1.66 (s, 1H), 1.56 (dd, J=13.2, 5.9 Hz, 6H).

### Example 145 and 146

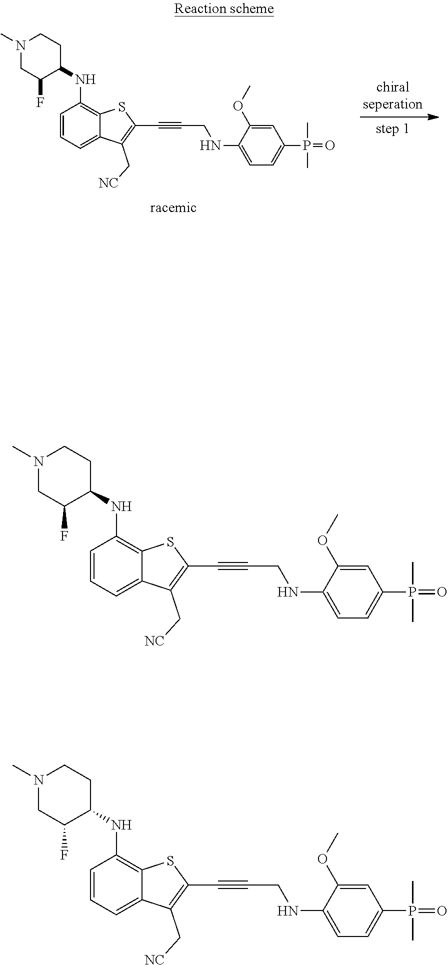 [SMILES: COc1cc(P(C)(C)=O)ccc1NCC#Cc1sc2c(N[C@@H]3CCN(C)C[C@@H]3F)cccc2c1CC#N ; COc1cc(P(C)(C)=O)ccc1NCC#Cc1sc2c(N[C@@H]3CCN(C)C[C@@H]3F)cccc2c1CC#N ; COc1cc(P(C)(C)=O)ccc1NCC#Cc1sc2c(N[C@H]3CCN(C)C[C@H]3F)cccc2c1CC#N]

### Experimental Details

### Step 1. 2-(2-(3-((4-(dimethylphosphoryl)-2-methoxyphenyl)amino)prop-1-yn-1-yl)-7-(((3S,4R)-3-fluoro-1-methylpiperidin-4-yl)amino)benzo[b]thiophen-3-yl)acetonitrile (145) and 2-(2-(3-((4-(dimethylphosphoryl)-2-m ethoxyphenyl)amino)prop-1-yn-1-yl)-7-(((3R,4S)-3-fluoro-1-methylpiperidin-4-yl)amino)benzo[b]thiophen-3-yl)acetonitrile (146)

The racemic sample 2-(2-(3-((4-(dimethylphosphoryl)-2-methoxyphenyl)amino)prop-1-yn-1-yl)-7-(((Z)-3-fluoro-1-methylpiperidin-4-yl)amino)benzo[b]thiophen-3-yl)acetonitrile (192 mg, 0.35 mmol) was separated by pre-CHIRAL-HPLC (Column:SC, Mobile Phase A:HeX:DCM=3:1)(0.2% IPA.M); Mobile Phase B: EtOH; A: B (V:V=50:50); 220 nm; Flow rate: 20 mL/min; RT: 10.612 min) to provide 52.00 mg (27.08% yield) of 2-(2-(3-((4-(dimethylphosphoryl)-2-methoxyphenyl)amino)prop-1-yn-1-yl)-7-(((3R,4S)-3-fluoro-1-methylpiperidin-4-yl)amino)benzo[b]thiophen-3-yl)acetonitrile (146) as off-white solid. LCMS: m/z=539 [M+1]⁺

¹H NMR (400 MHz, DMSO-d₆) δ 7.35-7.28 (m, 3H), 7.15 (d, J=11.8 Hz, 1H), 6.92-6.86 (m, 1H), 6.81 (d, J=7.8 Hz, 1H), 6.10 (t, J=6.3 Hz, 1H), 5.28 (d, J=8.5 Hz, 1H), 4.80 (d, J=50.2 Hz, 1H), 4.36 (d, J=6.3 Hz, 2H), 4.11 (s, 2H), 3.86 (s, 3H), 3.66 (d, J=29.6 Hz, 1H), 3.04 (t, J=10.5 Hz, 1H), 2.80 (d, J=10.2 Hz, 1H), 2.33-2.22 (m, 1H), 2.19 (s, 3H), 2.09 (t, J=11.7 Hz, 1H), 2.02-1.90 (m, 1H), 1.71 (d, J=11.6 Hz, 1H), 1.59 (d, J=13.2 Hz, 6H).

The racemic sample 2-(2-(3-((4-(dimethylphosphoryl)-2-methoxyphenyl)amino)prop-1-yn-1-yl)-7-(((Z)-3-fluoro-1-methylpiperidin-4-yl)amino)benzo[b]thiophen-3-yl)acetonitrile (192 mg, 0.35 mmol) was separated by pre-CHIRAL-HPLC (Column:SC, Mobile Phase A:HeX:DCM=3:1)(0.2% IPA.M); Mobile Phase B: EtOH; A: B (V:V=50:50); 220 nm; Flow rate: 20 mL/min; RT: 13.619 min) to provide 46.00 mg (23.95% yield) of 2-(2-(3-((4-(dimethylphosphoryl)-2-methoxyphenyl)amino)prop-1-yn-1-yl)-7-(((3S,4R)-3-fluoro-1-methylpiperidin-4-yl)amino)benzo[b]thiophen-3-yl)acetonitrile (145) as white solid. LCMS: m/z=539 [M+1]⁺

¹H NMR (400 MHz, DMSO-d₆) δ 7.35-7.27 (m, 3H), 7.15 (d, J=11.8 Hz, 1H), 6.92-6.89 (m, 1H), 6.81 (d, J=7.8 Hz, 1H), 6.10 (t, J=6.3 Hz, 1H), 5.28 (d, J=8.5 Hz, 1H), 4.80 (d, J=49.9 Hz, 1H), 4.36 (d, J=6.3 Hz, 2H), 4.11 (s, 2H), 3.86 (s, 3H), 3.66 (d, J=29.5 Hz, 1H), 3.03 (t, J=10.7 Hz, 1H), 2.80 (d, J=10.1 Hz, 1H), 2.28 (d, J=13.0 Hz, 1H), 2.19 (s, 3H), 2.12-2.08 (m, 1H), 2.02-1.92 (m, 1H), 1.75-1.65 (m, 1H), 1.59 (d, J=13.2 Hz, 6H).

### Example 147&148

### (4-((3-(7-(((3S,4R)-3-fluoro-1-(tetrahydro-2H-pyran-4-yl)piperidin-4-yl)amino)-3-(2,2,2-trifluoroethyl)benzo[b]thiophen-2-yl)prop-2-yn-1-yl)amino)-3-methoxyphenyl)dimethylphosphine oxide (147) and (4-((3-(7-(((3R,4S)-3-fluoro-1-(tetrahydro-2H-pyran-4-yl)piperidin-4-yl)amino)-3-(2,2,2-trifluoroethyl)benzo[b]thiophen-2-yl)prop-2-yn-1-yl)amino)-3-methoxyphenyl)dimethylphosphine oxide (148)

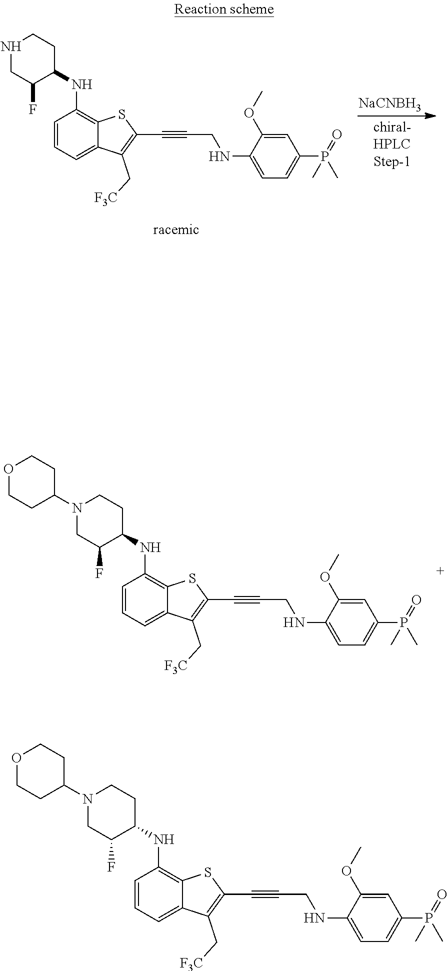 [SMILES: COc1cc(P(C)(C)=O)ccc1NCC#Cc1sc2c(N[C@@H]3CCNC[C@@H]3F)cccc2c1CC(F)(F)F ; "NaCNBH3" ; COc1cc(P(C)(C)=O)ccc1NCC#Cc1sc2c(N[C@@H]3CCN(C4CCOCC4)C[C@@H]3F)cccc2c1CC(F)(F)F ; COc1cc(P(C)(C)=O)ccc1NCC#Cc1sc2c(N[C@H]3CCN(C4CCOCC4)C[C@H]3F)cccc2c1CC(F)(F)F]

### Experimental Details

### Step 1. (4-((3-(7-(((3S,4R)-3-fluoro-1-(tetrahydro-2H-pyran-4-yl)piperidin-4-yl)amino)-3-(2,2,2-trifluoroethyl)benzo[b]thiophen-2-yl)prop-2-yn-1-yl)amino)-3-methoxyphenyl)dimethylphosphine oxide (147) and (4-((3-(7-(((3R,4S)-3-fluoro-1-(tetrahydro-2H-pyran-4-yl)piperidin-4-yl)amino)-3-(2,2,2-trifluoroethyl)benzo[b]thio phen-2-yl)prop-2-yn-1-yl)amino)-3-methoxyphenyl)dimethylphosphine oxide (148)

Into a 100-mL three necked bottle purged and maintained with an inert atmosphere of nitrogen, was placed (4-((3-(7-(((Z)-3-fluoropiperidin-4-yl)amino)-3-(2,2,2-trifluoroethyl)benzo[b]thiophen-2-yl)prop-2-yn-1-yl)amino)-3-methoxyphenyl)dimethylphosphine oxide (racemic) (0.152 g, 0.27 mmol), tetrahydro-4H-pyran-4-one (0.152 g, 1.52 mmol), NaCNBH₃ (0.208 g, 4.85 mmol), MeOH (5 mL). The reaction mixture was stirred at 50° C. for 6 h. The reaction mixture was purified by C18 column eluted with ACN/H₂O (V/V=4/6). The racemic sample (4-((3-(7-(((Z)-3-fluoro-1-(tetrahydro-2H-pyran-4-yl)piperidin-4-yl)amino)-3-(2,2,2-trifluoroethyl) benzo[b]thiophen-2-yl)prop-2-yn-1-yl)amino)-3-methoxyphenyl)dimethylphosphine oxide was separated by prep-chiral HPLC (Column IG, Mobile Phase A: n-Hexane Mobile Phase B: DCM; Flow rate: 20 mL/min; EtOH=50:50, 220 nm: RT: 8.904 min) to provide 0.040 g (22.92% yield) (4-((3-(7-(((3S,4R)-3-fluoro-1-(terrahydro-2H-pyran-4-yl)piperidin-4-yl)amino)-3-(2,2,2-trifluoroethyl)benzo[b]thiophen-2-ylprop-2-yn-1-l)amino)-3-methoxyphenyl)dimethylphosphine oxide (147) as white solid. LCMS: m/z=652 [M+1]⁺.

¹H NMR (400 MHz, MeOD) δ 7.32-7.24 (m, 2H), 7.19 (dd, J=8.1, 4.5 Hz, 2H), 6.93 (dd, J=8.1, 3.3 Hz, 1H), 6.76 (d, J=7.7 Hz, 1H), 4.77 (s, 1H), 4.36 (s, 2H), 3.98 (dd, J=11.3, 3.5 Hz, 2H), 3.92 (s, 3H), 3.76-3.59 (m, 3H), 3.37 (dd, J=23.5, 11.9 Hz, 2H), 3.01 (d, J=11.4 Hz, 1H), 2.60-2.48 (m, 2H), 2.46-2.32 (m, 2H), 1.98-1.89 (m, 2H), 1.85-1.77 (m, 2H), 1.75 (s, 3H), 1.71 (s, 3H), 1.65-1.47 (m, 2H).

The racemic sample (4-((3-(7-(((Z)-3-fluoro-1-(tetrahydro-2H-pyran-4-yl)piperidin-4-yl)amino)-3-(2,2,2-trifluoroethyl)benzo[b]thiophen-2-yl)prop-2-yn-1-yl)amino)-3-methoxyphenyl)dimethylphosphine oxide was separated by prep-chiral HPLC (Column:IG, Mobile Phase A: n-Hexane, Mobile Phase B: DCM: Flow rate: 20 mL/min; EtOH=50:50:220 nm: RT: 10.397 min) to provide 0.053 g (30.37% yield) (4-((3-(7-(((3R,4S)-3-fluoro-1-(tetrahydro-2H-pyran-4-yl)piperidin-4-yl)amino)-3-(2,2,2-trifluoroethyl)benzo[b]thiophen-2-yl)prop-2-yn-1-yl)amino)-3-methoxyphenyl)dimethylphosphine oxide (148) as white solid. LCMS: m/z=652 [M+1]⁺.

¹H NMR (400 MHz, DMSO) δ 7.39-7.07 (m, 4H), 6.91-6.69 (m, 2H), 6.05 (s, 1H), 5.10 (s, 1H), 4.79 (d, J=49.4 Hz, 1H), 4.33 (s, 2H), 3.87 (s, 2H), 3.84 (d, J=9.3 Hz, 3H), 3.79-3.55 (m, 2H), 3.32 (s, 4H), 3.26 (d, J=10.2 Hz, 1H), 3.12 (d, J=9.1 Hz, 1H), 2.89 (s, 1H), 2.37-2.21 (m, 1H), 1.99-1.70 (m, 3H), 1.61-1.50 (m, 6H), 1.48-1.32 (m, 2H), 1.23 (s, 1H).

### Example 149

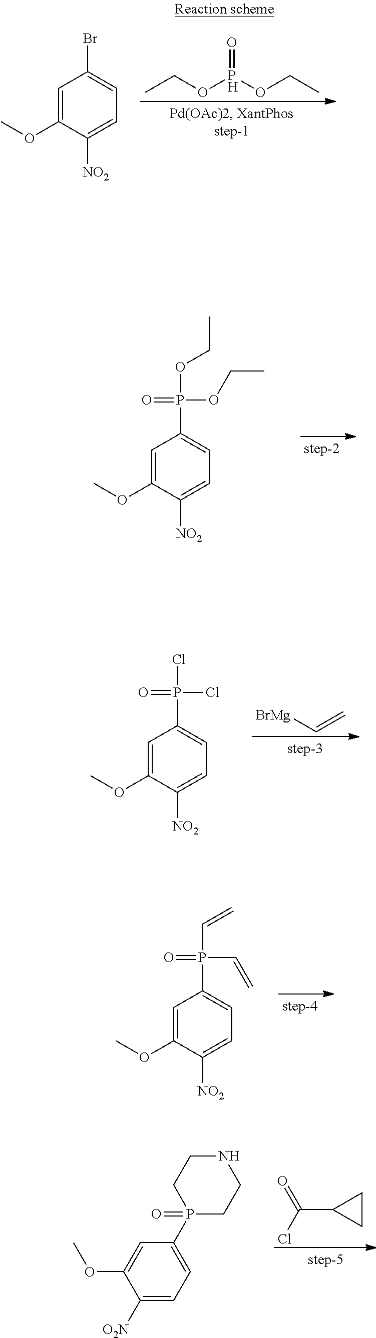

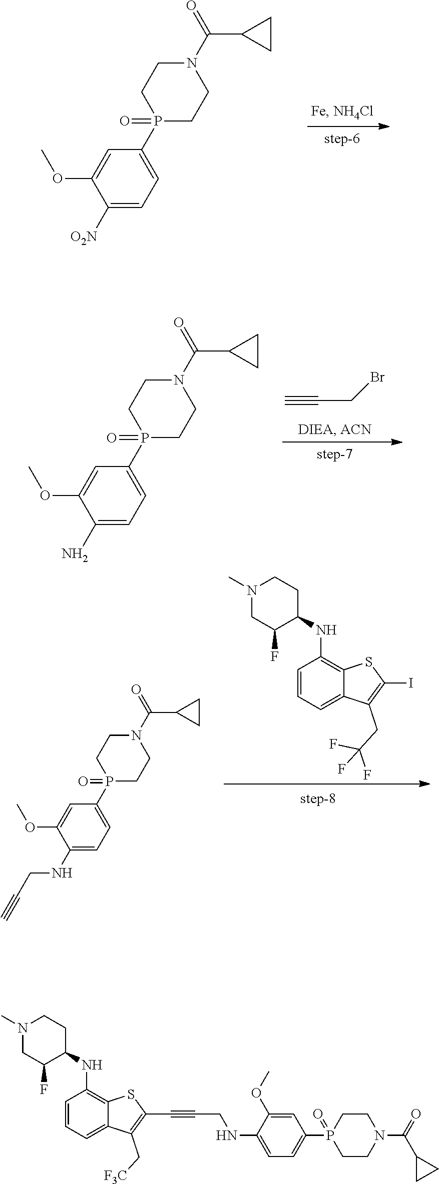

### Experimental Details

### Step 1. diethyl (3-methoxy-4-nitrophenyl)phosphonate

Into a 250-mL three necked bottle purged and maintained with an inert atmosphere of nitrogen, was placed 4-bromo-2-methoxy-1-nitrobenzene (15.12 g, 65.16 mmol), Pd(OAc)₂ (1.59 g, 7.08 mmol), Xantphos (7.63 g, 13.18 mmol), DIEA (25.42 g, 196.68 mmol), DMF (60 mL) and stirred at 110° C. for 5 min, then added diethyl phosphonate (12.90 g, 93.41 mmol) in DMF (5 mL). The reaction mixture was stirred at 110° C. for 2 h. The reaction mixture was filtered. The residue was concentrated under vacuum. The residue was applied onto a C₁₈ column eluted with ACN/H₂O (0.01% TFA) (v:v=1:1), Flow rate: 100 mL/min. This resulted in 16.48 g (87.44% yield) of diethyl (3-methoxy-4-nitrophenyl)phosphonate as brown oil. LCMS: m/z=290 [M+1]⁺

### Step 2. (3-methoxy-4-nitrophenyl)phosphonic dichloride

Into a 100-mL three necked bottle purged and maintained with an inert atmosphere of nitrogen, was placed diethyl (3-methoxy-4-nitrophenyl)phosphonate (1.09 g, 3.76 mmol), DMF (0.33 g, 4.51 mmol), SOCl₂ (12.86 g, 108.09 mmol). The reaction mixture was stirred at 80° C. for 12 h. The resulting solution was concentrated under vacuum. This resulted in 1.16 g (113.99% yield) of (3-methoxy-4-nitrophenyl)phosphonic dichloride as yellow oil. LCMS: m/z=270 [M+1]⁺

### Step 3. (3-methoxy-4-nitrophenyl)divinylphosphine oxide

Into a 250-mL three necked bottle purged and maintained with an inert atmosphere of nitrogen, was placed (3-methoxy-4-nitrophenyl)phosphonic dichloride (10.52 g, 38.95 mmol), THF (100 mL). The reaction mixture was cooled to −50° C. vinylmagnesium bromide (1 M in THF, 60 mL, 60 mmol) was added and stirred at −50° C.˜−20° C. for 3 h. The resulting solution was quenched with water (100 mL) and extracted with EA (2×100 mL). The organic layers combined and washed with brine (100 mL), dried over anhydrous Na₂SO₄, the residue was concentrated under vacuum. The residue was applied onto a C₁₈ column eluted with ACN/H₂O (0.01% TFA) (v:v=1:1), Flow rate: 100 m/min. This resulted in 8.92 g (90.46% yield) of (3-methoxy-4-nitrophenyl)divinylphosphine oxide as brown oil. LCMS: m/z=254 [M+1]⁺

### Step 4. 4-(3-methoxy-4-nitrophenyl)-1,4-azaphosphinane 4-oxide

Into a 100-mL reaction vial purged and maintained with an inert atmosphere of nitrogen, was placed (3-m ethoxy-4-nitrophenyl)divinylphosphine oxide (4.18 g, 16.52 mmol), THF (30 mL), NH₃ (7 M in MeOH, 10 mL). The reaction mixture was stirred at 100° C. for 3 h. The resulting solution was concentrated under vacuum. The residue was applied onto a C₁₈ column eluted with ACN/H₂O (0.01% NH₃OH) (v:v=1:1), Flow rate: 100 mL/min. This resulted in 522.00 mg (11.69% yield) of 4-(3-methoxy-4-nitrophenyl)-1,4-azaphosphinane 4-oxide as brown oil. LCMS: m/z=271 [M+1]⁺

### Step 5. cyclopropyl(4-(3-methoxy-4-nitrophenyl)-4-oxido-1,4-azaphosphinan-1-yl)methanone

Into a 250-mL three necked bottle purged and maintained with an inert atmosphere of nitrogen, was placed 4-(3-methoxy-4-nitrophenyl)-1,4-azaphosphinane 4-oxide (480.00 mg, 1.77 mmol), DIEA (1.17 g, 9.09 mmol), DCM (1 mL). The reaction mixture was cooled to 0° C. before cyclopropanecarbonyl chloride (509.00 mg, 4.86 mmol) was added. The reaction mixture was stirred at room temperature for 2 h. The resulting solution was quenched with water (50 mL) and extracted with EA (2×100 mL). The organic layers combined and washed with brine (30 mL), dried over anhydrous Na₂SO₄, the residue was concentrated under vacuum. The residue was applied onto a silica gel column eluted with EA:hexane (v:v=1:1). This resulted in 550.00 mg (91.52% yield) of cyclopropyl(4-(3-methoxy-4-nitrophenyl)-4-oxido-1,4-azaphosphinan-1-yl)methanone as brown oil. LCMS: m/z=339 [M+1]⁺

### Step 6. (4-(4-amino-3-methoxyphenyl)-4-oxido-1,4-azaphosphinan-1-yl)(cyclopropyl)methanone

Into a 100-mL three necked bottle purged and maintained with an inert atmosphere of nitrogen, was placed cyclopropyl(4-(3-methoxy-4-nitrophenyl)-4-oxido-1,4-azaphosphinan-1-yl)methanone (500.00 mg, 1.47 mmol), Fe (510.00 mg, 9.13 mmol), NH₄Cl (518.00 mg, 9.68 mmol), ETOH (10 mL), H₂O (2 mL). The reaction mixture was stirred at 80° C. for 2 h. The reaction mixture was filtered and the filter cake was washed with EtOH (20 mL). The residue was concentrated under vacuum. The residue was applied onto a C₁₈ column eluted with ACN/H₂O (0.01% TFA) (v:v=1:1), Flow rate: 100 mL/min. This resulted in 454.00 mg (99.63% yield) of (4-(4-amino-3-methoxyphenyl)-4-oxido-1,4-azaphosphinan-1-yl)(cyclopropyl)methanone as brown oil. LCMS: m/z=309 [M+1]⁺

### Step 7. cyclopropyl(4-(3-methoxy-4-(prop-2-yn-1-ylamino)phenyl)-4-oxido-1,4-azaphosphinan-1-yl)methanone

Into a 50-mL three necked bottle purged and maintained with an inert atmosphere of nitrogen, was placed (4-(4-amino-3-methoxyphenyl)-4-oxido-1,4-azaphosphinan-1-yl)(cyclopropyl)methanone (41.011 mg, 1.33 mmol), 3-bromoprop-1-yne (522.00 mg, 4.38 mmol), DIEA (504.00 mg, 3.90 mmol), MeCN (10 mL). The reaction mixture was stirred at 80° C. for 5 h. The residue was concentrated under vacuum. The residue was app lied onto a C₁₈ column eluted with ACN/H₂O (0.01% TFA) (v:v=1:1), Flow rate: 80 mL/min. This resulted in 287.00 mg (62.31% yield) of cyclopropyl(4-(3-methoxy-4-(prop-2-yn-1-ylamino)phenyl)-4-oxido-1,4-azaphosphinan-1-yl)methanone as brown oil. LCMS: m/z=347 [M+1]⁺

### Step 8. cyclopropyl(4-(4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)-4-oxido-1,4-azaphosphinan-1-yl)methanone (149)

Into a 50-mL three necked bottle purged and maintained with an inert atmosphere of nitrogen, was placed (3S,4R)-3-fluoro-N-(2-iodo-3-(2,2,2-trifluoroethyl)benzo[b]thiophen-7-yl)-1-methylpiperidin-4-amine (106 mg, 0.22 mmol), cyclopropyl(4-(3-methoxy-4-(prop-2-yn-1-ylamino)phenyl)-4-oxido-1,4-azaphosphinan-1-yl)methano ne (226.00 mg, 0.65 mmol), Pd(PPh₃)₂Cl₂ (23.00 mg, 0.032 mmol), CuI (25.00 mg, 0.13 mmol), DIEA (109.00 mg, 0.84 mmol), DMSO (8 mL). The reaction mixture was stirred at 50° C. for 3 h. The residue was applied onto a C₁₈ column eluted with ACN/H₂O (0.01% TFA) (v:v=1:1), Flow rate: 80 mL/min. the residue was concentrated under vacuum. The resulting crude product was further purified by pre-HPLC with ACN/H₂O (0.1% ammonium hydroxide), Flow rate: 70 mL/min; Gradient: 35-70-90% B (2-30-60 min); 268 nm; RT: 29.238-31.270). This resulted in 49.00 mg (31.60% yield) of cyclopropyl(4-(4-((3-(7-(((3S,4R)-3-fluoro-1-m ethylpiperidin-4-yl)amino)-3-(2,2,2-trifluoroethyl)benzo[b]thiophen-2-yl)prop-2-yn-1-yl)amino)-3-methoxyphenyl)-4-oxido-1,4-azaphosphinan-1-yl)methanone (149) as white solid. LCMS: m/z=691 [M+1]⁺

¹H NMR (400 MHz, DMSO-d₆) δ 7.32-7.25 (m, 2H), 7.23-7.13 (m, 2H), 6.90-6.87 (m, 1H), 6.78 (d, J=7.7 Hz, 1H), 6.21 (t, J=6.3 Hz, 1H), 5.19 (d, J=8.5 Hz, 1H), 4.89-4.70 (m, 1H), 4.39-4.32 (m, 2H), 4.25-4.11 (m, 2H), 4.00-3.74 (m, 6H), 3.68-3.61 (m, 1H), 3.48-3.41 (m, 1H), 3.10-2.95 (m, 1H), 2.80 (d, J=10.8 Hz, 1H), 2.36-2.00 (m, 8H), 1.99-1.65 (m, 4H), 0.89-0.77 (m, 4H).

### Example 150

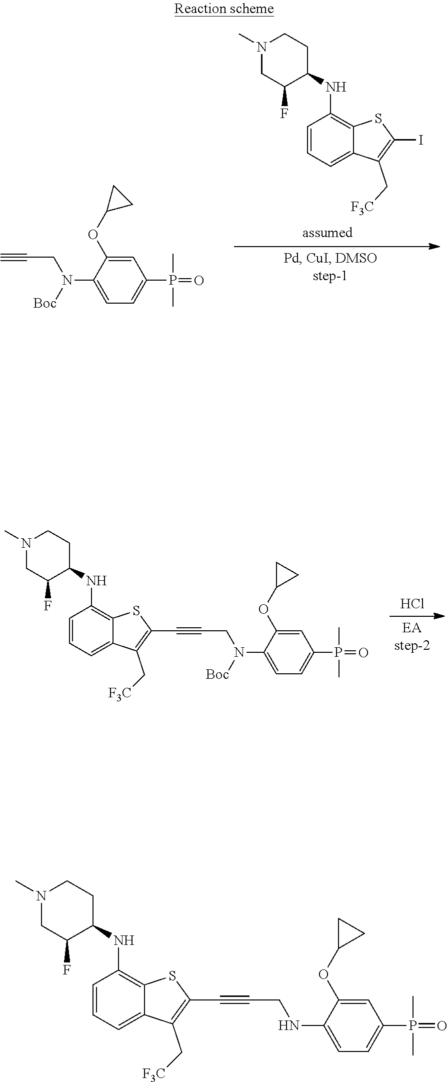 [SMILES: C#CCN(C(=O)OC(C)(C)C)c1ccc(P(C)(C)=O)cc1OC1CC1 ; CN1CC[C@@H](Nc2cccc3c(CC(F)(F)F)c(I)sc23)[C@@H](F)C1 ; CN1CC[C@@H](Nc2cccc3c(CC(F)(F)F)c(C#CCN(C(=O)OC(C)(C)C)c4ccc(P(C)(C)=O)cc4OC4CC4)sc23)[C@@H](F)C1 ; Cl ; CN1CC[C@@H](Nc2cccc3c(CC(F)(F)F)c(C#CCNc4ccc(P(C)(C)=O)cc4OC4CC4)sc23)[C@@H](F)C1]

### Experiment Details

### Step 1 tert-butyl (2-cyclopropoxy-4-(dimethylphosphoryl)phenyl)(3-(7-(((3S,4R)-3-fluoro-1-methylpiperidin-4-yl)amino)-3-(2,2,2-trifluoroethyl)benzo[b]thiophen-2-yl)prop-2-yn-1-yl)carbamate

Into a 10-mL round-bottom flask was placed (3S,4R)-3-fluoro-N-(2-iodo-3-(2,2,2-trifluoroethyl)benzo[b]thiophen-7-yl)-1-(tetrahydro-2H-pyran-4-yl)piperidin-4-amine (0.606 g, 1.28 mmol), tert-butyl (2-cyclopropoxy-4-(dimethylphosphoryl)phenyl)(prop-2-yn-1-yl)carbamate (0.642 g, 1.76 mmol), Pd(PPh₃)₂Cl₂ (0.184 g, 260.65 μmol), CuI (0.069 g, 362.29 umol), DIEA (0.635 g, 4.91 mmol), methyl sulfoxide (8 mL). The reaction was stirred under nitrogen atmosphere at RT for 4 h. The reaction mixture was filtered through celite pad and c elite pad was washed with ACN (3×20 mL). The filterate was then concentrated under reduced pressure to afford the crude product. The crude product was purified by C18 column eluted with ACN/H₂O (v/v=2/3), filtered and concentrated under vacuum. This resulted in 0.82 g (90.29% yield) of tert-butyl (2-cyclopropoxy-4-(dimethylphosphoryl)phenyl)(3-(7-(((3S,4R)-3-fluoro-1-methylpiperidin-4-yl)amino)-3-(2,2,2-trifluoroethyl)benzo[b]thiophen-2-yl)prop-2-yn-1-yl)carbamate as yellow solid. LCMS: m/z=708 [M+1]⁺.

### Step 2. (3-cyclopropoxy-4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)phenyl)dimethylphosphine oxide (150)

Into a 10-mL round-bottom flask purged and maintained with an inert atmosphere of nitrogen, was placed tert-butyl (2-cyclopropoxy-4-(dimethylphosphoryl)phenyl)(3-(7-(((3S,4R)-3-fluoro-1-methylpiperidin-4-yl)amino)-3-(2,2,2-trifluoroethyl)benzo[b]thiophen-2-yl)prop-2-yn-1-yl)carbamate (0.812 g, 1.14 mmol), ethyl acetate (5 mL) and HCl in EA solution (5 mL, 4 M). The reaction mixture was stirred at room temperature for 1 h. The mixture was concentrated to afford the crude product. The crude product was purified by preparative HPLC (Mobile Phase A: water (0.1% ammonium hydroxide), Mobile Phase B: ACN; Flow rate: 70 mL/min; Gradient: 40-70-70% B (2-30-35 min); 264 nm; 29.50-33.77) to provide the desired product. This resulted in 0.578 g (82.92% yield) of (3-cyclopropoxy-4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)phenyl)dimethylphosphine oxide (150) as white solid. LCMS: m/z=608 [M+1]⁺.

¹H NMR (400 MHz, DMSO-d₆) δ 7.43 (d, J=11.9 Hz, 1H), 7.37-7.13 (m, 3H), 6.81-6.78 (m, 2H), 5.91 (d, J=6.8 Hz, 1H), 5.13 (d, J=8.5 Hz, 1H), 4.79 (d, J=49.4 Hz, 1H), 4.32 (d, J=6.4 Hz, 2H), 4.01-3.55 (m, 4H), 3.03 (t, J=11.4 Hz, 1H), 2.79 (d, J=10.8 Hz, 1H), 2.30-1.89 (m, 6H), 1.71 (d, J=12.4 Hz, 1H), 1.58 (d, J=13.1 Hz, 6H), 0.89-0.65 (m, 4H).

### Example 151

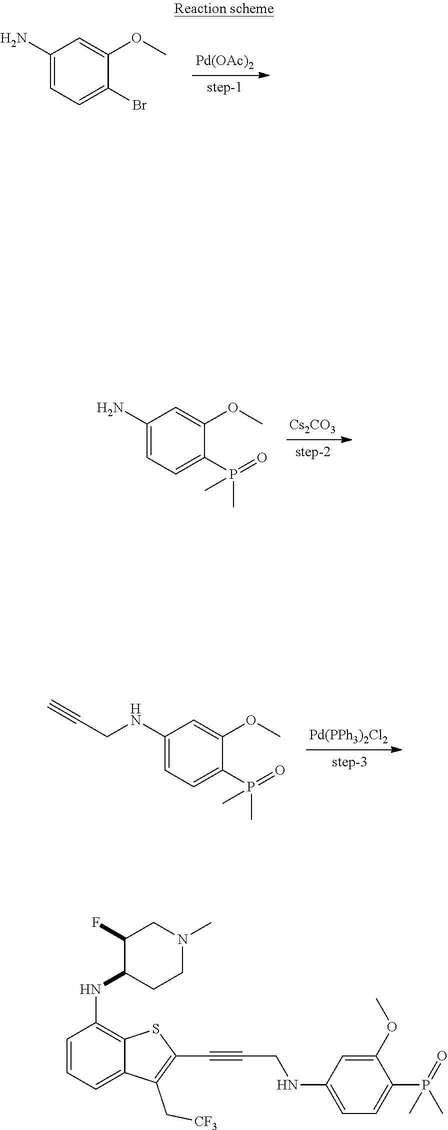

### Experimental Details

### Step 1. (4-amino-2-methoxyphenyl)dimethylphosphine oxide

Into a 100-mL three necked bottle purged and maintained with an inert atmosphere of nitrogen, was placed 4-bromo-3-methoxyaniline (4.03 g, 19.94 mmol), Pd(OAc)₂ (0.95 g, 4.23 mmol), Xantphos (1.26 g, 2.18 mmol), DIEA (3.05 g, 23.60 mmol) and DMF (20 mL). The reaction mixture was stirred at 130° C. for 0.5 h. Then was added Dimethylphosphine oxide (3.58 g, 45.87 mmol). The reaction mixture was stirred at 130° C. for another 1 h. LCMS showed that the reaction was completed. The reaction mixture was purified by C18 column eluted with ACN/H₂O (v/v=2/8). This resulted in 2.31 g (58.14% yield) of (4-amino-2-methoxyphenyl)dimethylphosphine oxide as red oil. LCMS: m/z=200 [M+1]⁺.

### Step 2. (2-methoxy-4-(prop-2-yn-1-ylamino)phenyl)dimethylphosphine oxide

Into a 50-mL flask was placed (4-amino-2-methoxyphenyl)dimethylphosphine oxide (0.960 g, 4.82 mmol), 3-Bromopropyne (0.712 g, 5.98 mmol), K₂CO₃ (2.032 g, 6.24 mmol), KI (0.402 g, 2.42 mmol), DMF (5 mL). The reaction mixture was stirred at 80° C. for 4 h. LCMS showed that the reaction was completed. The reaction mixture was purified by C18 column eluted with ACN/H₂O (v/v=4/6). This resulted in 0.263 g (23.01% yield) of (2-methoxy-4-(prop-2-yn-1-ylamino)phenyl)dimethylphosphine oxide as red solid. LCMS: m/z=238 [M+1]⁺.

### Step 3. (4-((3-(7-(((Z)-3-fluoro-1-methylpiperidin-4-yl)amino)-3-(2,2,2-trifluoroethyl)benzo[b]thiophen-2-yl) prop-2-yn-1-yl)amino)-2-methoxyphenyl)dimethylphosphine oxide (racemic) (151)

Into a 50-mL three necked bottle and maintained with an inert atmosphere of nitrogen, was placed (2-meth oxy-4-(prop-2-yn-1-ylamino)phenyl)dimethylphosphine oxide (0.104 g, 0.44 mmol), (Z)-3-fluoro-N-(2-iodo-3-(2,2,2-trifluoroethyl)benzo[b]thiophen-7-yl)-1-methylpiperidin-4-amine (racemic) (0.099 g, 0.21 mmol), CuI (0.042 g, 0.22 mmol), Pd(PPh₃)₂Cl₂ (0.081 g, 0.11 mmol), DIEA (0.095 g, 0.74 mmol) and DMSO (5 mL). The reaction mixture was stirred at room temperature for 1 h. LCMS showed that the reaction was completed. The mixture was purified by preparative HPLC (Mobile Phase A: water (10 mmoL/L ammonium hydr oxide), Mobile Phase B: ACN; Flow rate: 40 mL/min; Gradient: 40-65-65% B (2-30-60 min); 260 nm; RT: 28.885-31.049 min). This resulted in 0.025 g (20.51% yield) of (4-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-2-methoxyphenyl)dimethylphosphine oxide (racemic) (151) as off white solid. LCMS: m/z=582 [M+1]⁺.

¹H NMR (400 MHz, DMSO) δ 7.52-7.36 (m, 1H), 7.36-7.16 (m, 2H), 6.87-6.67 (m, 2H), 6.51-6.31 (m, 2H), 5.20 (d, J=8.5 Hz, 1H), 4.80 (d, J=49.4 Hz, 1H), 4.34 (d, J=6.0 Hz, 2H), 3.86 (d, J=10.8 Hz, 1H), 3.81 (s, 3H), 3.75-3.55 (m, 1H), 3.04 (t, J=10.8 Hz, 1H), 2.80 (d, J=10.6 Hz, 1H), 2.34-2.23 (m, 1H), 2.19 (s, 3H), 2.17-2.10 (m, 1H), 2.07 (d, J=11.1 Hz, 1H), 2.02-1.89 (m, 1H), 1.72 (d, J=10.4 Hz, 1H), 1.55 (s, 3H), 1.51 (s, 3H).

### Example 152

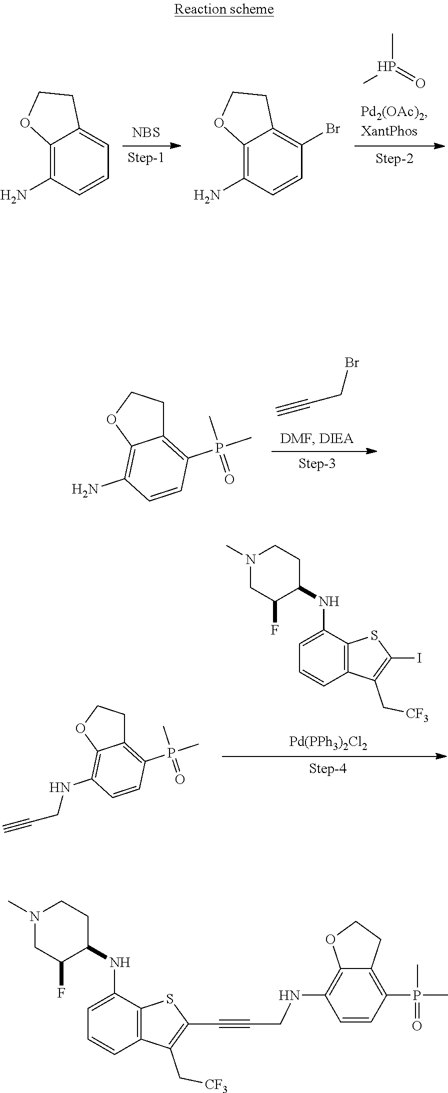

### Experiment Details

### Step 1. 4-bromo-2,3-dihydrobenzofuran-7-amine

Into a 500 mL flask was placed 2,3-dihydrobenzofuran-7-amine (9.969 g, 73.76 mmol), ACN (250 mL). The reaction was cooled to −20° C. and stirred. NBS (13.784 g, 77.45 mmol) was slowly added to the solution and warmed to room temperature naturally. The solution was stirred overnight. The reaction was quenched by water 10 mL and concentrated to 100 mL of total volume. 100 mL of water was add to the concentrated solution, and precipitates was start to form. The solid was filtered. The filter cake was washed with 100 mL of water, and stirred with dilute Na₂S₂O₄ aqueous solution. The slurry solution was filtered again and was with water 100 mL. The filter cake was stoved to afford 4-bromo-2,3-dihydrobenzofuran-7-amine (14.16 g, 66.15 mmol, 89.69% yield) as brown solid. LCMS: m/z=214[M+1]⁺.

### Step 2. (7-amino-2,3-dihydrobenzofuran-4-yl)dimethylphosphine oxide

Into a 500 mL flask purged and maintained with N₂ was placed 4-bromo-2,3-dihydrobenzofuran-7-amine (14.08 g, 65.78 mmol), Palladium (II) acetate, (1.47 g, 6.55 mmol), XantPhos (7.87 g, 13.60 mmol), DIEA (17.30 g, 133.86 mmol), DMF (150 mL). The reaction was heated to 120° C. and dimethylphosphine oxide (8.07 g, 103.40 mmol) in DMF (10 mL) was add to the solution while stirring. The solution was stirred for 1 h. The solution was cooled to room temperature, and poured to water 250 mL. The solid was filtered, and the filter cake was washed with water 50 mL. The filtration was concentrate under vacuum and purified with C₁₈ column, eluted with ACN/water (v/v=3/10) to afford (7-amino-2,3-dihydrobenzofuran-4-yl) dimethylphosphine oxide (9.13 g, 43.23 mmol, crude) as off-white solid. LCMS: m/z=212[M+1]⁺

### Step 3. dimethyl(7-(prop-2-yn-1-ylamino)-2,3-dihydrobenzofuran-4-yl)phosphine oxide

Into a 250 mL flask was placed (7-amino-2,3-dihydrobenzofuran-4-yl)dimethylphosphine oxide (0.192 g, 332.39 μmol), 3-bromoprop-1-yne (3.44 g, 28.92 mmol), DIEA (4.83 g, 37.37 mmol), DMF (50 mL). The reaction was stirred overnight at 80° C. The reaction was purified with C₁₈ column, eluted with ACN/water (v/v=1/3) to afford dimethyl(7-(prop-2-yn-1-ylamino)-2,3-dihydrobenzofuran-4-yl)phosphine oxide (1.782 g, 7. mmol, 29.90% yield) as white solid. LCMS: m/z=250 [M−1]⁺

### Step 4. (7-((3-(7-(((Z)-3-fluoro-1-methylpiperidin-4-yl)amino)-3-(2,2,2-trifluoroethyl)benzo[b]thiophen-2-yl) prop-2-yn-1-yl)amino)-2,3-dihydrobenzofuran-4-yl)dimethylphosphine oxide (152)

Into a 4 mL vial purged and maintained with nitrogen atmosphere was placed (Z)-3-fluoro-N-(2-iodo-3-(2,2,2-trifluoroethyl)benzo[b]thiophen-7-yl)-1-methylpiperidin-4-amine (0.050 g, 105.87 μmol), dimethyl(7-(prop-2-yn-1-ylamino)-2,3-dihydrobenzofuran-4-yl)phosphine oxide (0.038 g, 152.46 μmol), Pd(PPh₃)₂Cl₂ (0.010 g, 14.17 μmol), CuI (0.007 g, 36.76 μmol), TEA (0.025 g, 247.06 μmol), DMF (1 mL), and stirred overnight at 50 gh The reaction was diluted with water (2 mL), extracted with EA (2 mL×2). The combined organic layers were washed with water (2 mL) and brine (2 mL) successively, separated, then concentrated with vacuum. The residue was purified with prep-HPLC (Mobile Phase A: water (formic acid), Mobile Phase B: ACN; Flow rate: 70 m/min; Gradient: 40-63-65% B (2-30-34 min); 266 nm; RT: 30.28-32.23) to afford (7-((3-(7-(((Z)-3-fluoro-1-methylpiperidin-4-yl)amino)-3-(2,2,2-trifluoroethyl)benzo[b]thiophen-2-yl)prop-2-yn-1-yl)amino)-2,3-dihydrobenzofuran-4-yl)dimethylphosphine oxide (152) (0.013 g, 21.90 μmol, 20.69% yield) as white solid. LCMS: m/z=594 [M+1]⁺.

### Example 153

### (5-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-6-methoxypyridin-2-yl)dimethylphosphine oxide (153)

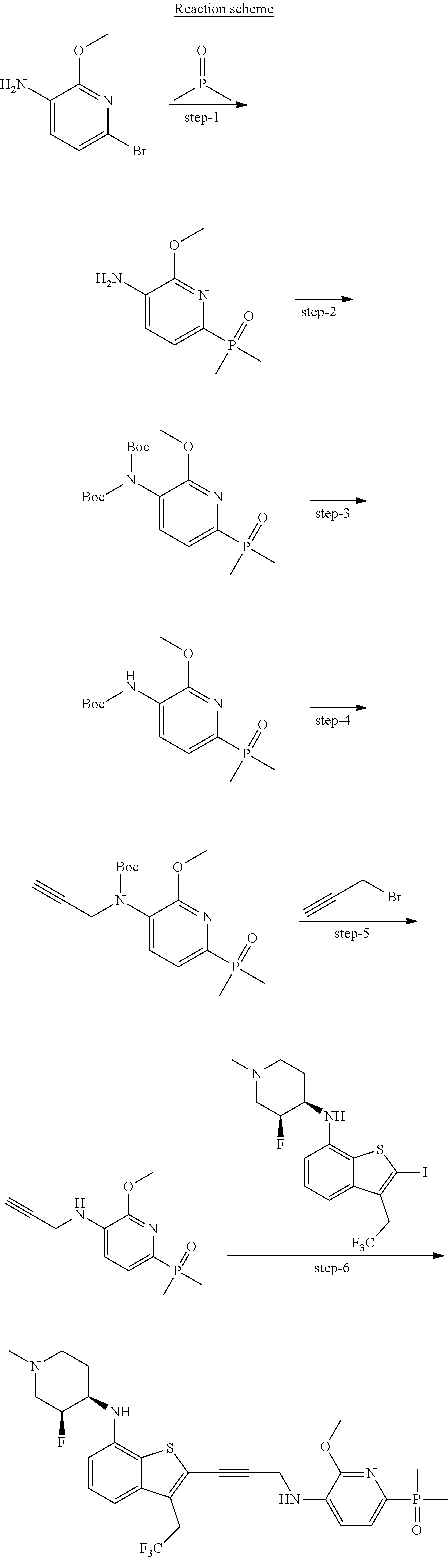

### Experiment Details

### Step 1. (5-amino-6-methoxypyridin-2-yl)dimethylphosphine oxide

Into a 500 mL three rounds bottom flask was placed 6-bromo-2-methoxypyridin-3-amine (21.86 g, 107.66 mmol), Palladium (II) acetate (3.94 g, 17.56 mmol), Dimethylbisdiphenylphosphinoxanthene (11.22 g, 19.40 mmol), DIEA (39.70 g, 307.19 mmol), DMF (150 mL). The reaction was heat to 130° C. under nitrogen. Dimethylphosphine oxide (22.08 g, 282.90 mmol) was added to the reaction slowly. The mixture was stirred at 130° C. for 1 h. The residue purified by flash-C18 eluted with ACN/water (V/V=1/9) to provide the desired product. This resulted in 14.14 g (65.75% yield) of (5-amino-6-methoxypyridin-2-yl)dimethylphosphine oxide as off-white solid. LCMS: m/z=201 [M+1]⁺.

### Step 2. tert-butyl N-tert-butoxycarbonyl-N-(6-dimethylphosphoryl-2-methoxy-3-pyridyl)carbamate

Into a 500 mL round-bottom flask was placed (5-amino-6-methoxypyridin-2-yl)dimethylphosphine oxide (14.13 g, 70.59 mmol), Di-tert-butyl dicarbonate (54.46 g, 249.53 mmol), N-(4-pyridyl)dimethylamine (1.05 g, 8.59 mmol), 1,4-Dioxane (150 mL). The reaction was stirred at 100° C. for 1 h. The mixture was concentrated under vacuum. This resulted in 30.45 g (crude) of tert-butyl N-tert-butoxycarbonyl-N-(6-dimethylphosphoryl-2-methoxy-3-pyridyl)carbamate as brown oil. LCMS: m/z=401 [M+1]⁺.

### Step 3. tert-butyl (6-(dimethylphosphoryl)-2-methoxypyridin-3-yl)carbamate

Into a 500 mL round-bottom flask was placed tert-butyl N-tert-butoxycarbonyl-N-(6-dimethylphosphoryl-2-methoxy-3-pyridyl)carbamate (30.12 g, 75.22 mmol), K₂CO₃ (47.21 g, 341.59 mmol), MeOH (150 mL). The reaction was stirred at 70° C. for 1 h. The mixture was concentrated under vacuum. The reaction was quenched by water (300 mL), extracted with EA (2×200 mL). The organic layers were combined, washed with brine (2×500 mL), dried over anhydrous sodium sulfate, filtered and concentrated under vacuum. This resulted in 17.74 g (78.53% yield) of tert-butyl (6-(dimethylphosphoryl)-2-methoxypyridin-3-yl)carbamate as brown solid. LCMS: m/z=301 [M+1]⁺.

### Step 4. tert-butyl (6-(dimethylphosphoryl)-2-methoxypyridin-3-yl)(prop-2-yn-1-yl)carbamate

Into a 500 mL round-bottom flask was placed tert-butyl (6-(dimethylphosphoryl)-2-methoxypyridin-3-yl)carba mate (17.50 g, 58.28 mmol), THF (180 mL). The reaction was stirred at 0° C. NaH (11.94 g, 497.55 mmol) was added to the reaction slowly at 0° C. and stirred for 0.5 h. 3-bromoprop-1-yne (64.07 g, 538.59 mm ol) was added to the reaction and stirred at 60° C. overnight. The reaction was quenched by water (300 m L), extracted with EA (2×300 mL). The organic layers were concentrated under vacuum. The mixture w as purified by flash-C18 eluted with ACN/water (v/v=3/7) provide the desired product. This resulted in 11.66 g (59.14% yield) of tert-butyl (6-(dimethylphosphoryl)-2-methoxypyridin-3-yl)(prop-2-yn-1-yl)carbamate a yellow oil. LCMS: m/z=339 [M+1]⁺.

### Step 5. (6-methoxy-5-(prop-2-yn-1-ylamino)pyridin-2-yl)dimethylphosphine oxide

Into a 500 mL round-bottom flask was placed tert-butyl (6-(dimethylphosphoryl)-2-methoxypyridin-3-yl)(prop-2-yn-1-yl)carbamate (11.66 g, 34.46 mmol), TFA (30 mL), DCM (100 mL). The reaction was stirred at R.T for 0.5 h. The reaction was concentrated under vacuum. The residue purified by flash-C18 eluted with ACN/water (v/v=3/7) provide the desired product. This resulted in 6.23 g (crude) of (6-methoxy-5-(prop-2-yn-1-ylamino)pyridin-2-yl)dimethylphosphine oxide as brown solid. LCMS: m/z=239 [M+1]⁺.

### Step 6. (5-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-6-methoxypyridin-2-yl)dimethylphosphine oxide (153)

Into a 500 mL round-bottom flask was placed (3S,4R)-3-fluoro-N-(2-iodo-3-(2,2,2-trifluoroethyl)benzo[b]thiophen-7-yl)-1-methylpiperidin-4-amine (6.01 g, 12.72 mmol), (6-methoxy-5-(prop-2-yn-1-ylamino)pyridin-2-yl)dimethylphosphine (3.51 g, 14.73 mmol), bis(triphenylphosphine)palladium(II) chloride (1.41 g, 1.99 mmol), CuI (0.13 g, 0.70 mmol), DIEA (5.10 g, 39.44 mmol), Methyl sulfoxide (100 mL). The reaction was stirred at 60° C. for 6 h under nitrogen. The reaction was quenched with water (300 mL), extracted with EA (2×200 mL). The organic layers were combined, washed with brine (2×500 mL), dried over anhydrous sodium sulfate, filtered and concentrated under vacuum. The residue purified by flash-C18 eluted with ACN/water (V/V=1/4) to provide the desired product. The mixture was purified by preparative HPLC (Mobil e Phase A: water (10 mmol/L NH₄OH), Mobile Phase B: ACN; Flow rate: 70 mL/min; Gradient: 35-68-68% B (2-30-35 min); 266 nm; RT: 29.45-33.32 min) to provide the desired product. This resulted in 3.1254 g (42.17% yield) of (5-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-6-methoxypyridin-2-yl)dimethylphosphine oxide (153) as yellow solid. LCMS: m/z=583 [M+1]⁺.

¹H NMR (400 MHz, DMSO-d₆) δ 7.42-7.36 (m, 1H), 7.31-7.19 (m, 2H), 7.10-7.04 (m, 1H), 6.78 (d, J=7.6 Hz, 1H), 6.42 (t, J=6.3 Hz, 1H), 5.20 (d, J=8.5 Hz, 1H), 4.80 (d, J=49.5 Hz, 1H), 4.3 (d, J=6.3 Hz, 2H), 3.93 (s, 3H), 3.87-3.75 (m, 2H), 3.65 (d, J=28.9 Hz, 1H), 3.03 (t, J=11.2 Hz, 1H), 2.79 (d, J=11.0 Hz, 1H), 2.18 (m, 4H), 2.12-2.03 (m, 1H), 2.02-1.89 (m, 1H), 1.71 (d, J=12.5 Hz, 1H), 1.57 (d, J=13.4 Hz, 6H).

### Example 154

### (3-methoxy-4-((3-(7-((1-(tetrahydro-2H-pyran-4-yl)piperidin-4-yl)amino)-3-(2,2,2-trifluoroethyl)benzo[b]thio phen-2-yl)prop-2-yn-1-yl)amino)phenyl)dimethylphosphine oxide (154)

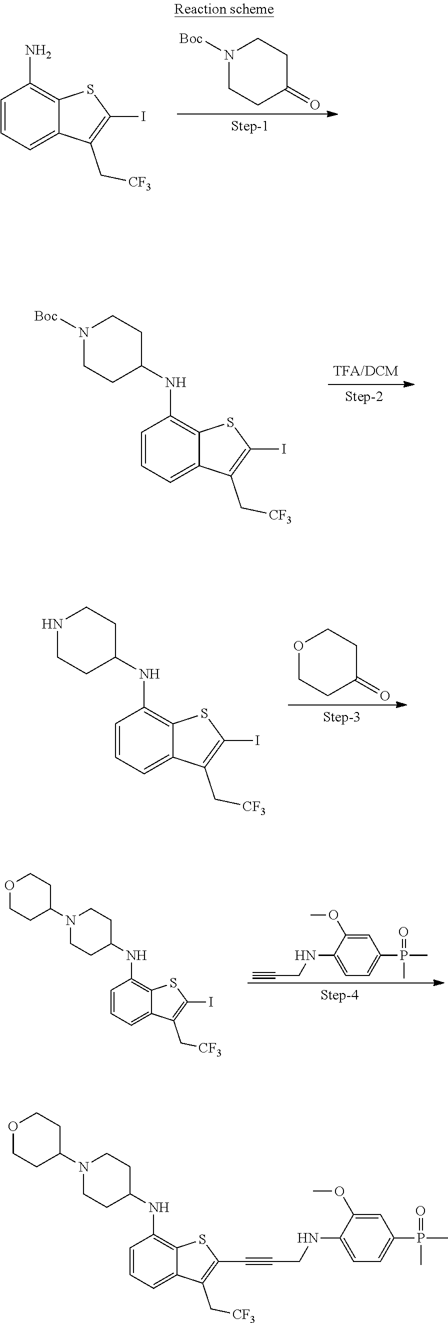

### Experiment Details

### Step 1. tert-butyl 4-((2-iodo-3-(2,2,2-trifluoroethyl)benzo[b]thiophen-7-yl)amino)piperidine-1-carboxylate

Into an opened 40 mL vail was placed tert-butyl tert-butyl 4-oxopiperidine-1-carboxylate (1.740 g, 8.73 m mol), 2-iodo-3-(2,2,2-trifluoroethyl)benzo[b]thiophen-7-amine (1.004 g, 2.81 mmol). The reaction was heated to 120° C. and stirred for 30 min then cooled to room temperature naturally. Sodium cyanoboronhydride, (0.736 g, 11.90 mmol), EtOH (10 mL), acetic acid (0.01 mL) was added to the solution. The reaction was stirred for 1.5 h at room temperature. LCMS monitored the reaction was complete. The reaction was diluted with water, extracted by EA (20 mL×2). The combined organic phase was concentrated under vacuum to afford crude tert-butyl 4-((2-iodo-3-(2,2,2-trifluoroethyl)benzo[b]thiophen-7-yl)amino)piperidine-1-carboxylate (0.153 g, 270.61 μmol, 9.63% yield) as off-white solid. LCMS: m/z=541[M+1]⁺.

### Step 2. N-(2-iodo-3-(2,2,2-trifluoroethyl)benzo[b]thiophen-7-yl)piperidin-4-amine

Into a 4 mL flask was placed tert-butyl tert-butyl 4-((2-iodo-3-(2,2,2-trifluoroethyl)benzo[b]thiophen-7-yl)amino)piperidine-1-carboxylate (0.643 g, 1.19 mmol), TFA (2 mL), DCM (10 mL). The reaction was stirred overnight at room temperature. LCMS showed the reaction was complete. The reaction quenched with saturated NaHCO₃ (aq.). The solution was diluted with water (20 mL), extracted with DCM (10 mL×2). The combined organic layers were washed with water (10 mL) and brine (10 mL) successively, separated, then concentrated with vacuum to afford crude N-(2-iodo-3-(2,2,2-trifluoroethyl)benzo[b]thiophen-7-yl)piperidin-4-amine (0.153 g, 328.84 μmol, 124.78% yield) as an off-white solid. LCMS: m/z=441[M+1]⁺

### Step 3. N-(2-iodo-3-(2,2,2-trifluoroethyl)benzo[b]thiophen-7-yl)-1-(tetrahydro-2H-pyran-4-yl)piperidin-4-amine

Into a 8 mL vail was placed N-(2-iodo-3-(2,2,2-trifluoroethyl)benzo[b]thiophen-7-yl)piperidin-4-amine (0.401 g, 910.82 μmol), tetrahydro-4H-pyran-4-one (0.242 g, 2.42 mmol), sodium cyanoboronhydride (0.288 g, 6.72 mmol), EtOH (2 mL), acetic acid (0.01 mL). The reaction was stirred overnight at 50° C. LCMS showed the reaction was complete. The residue was purified with C₁₈ column, eluted with ACN/water (v/v=2/3) to afford N-(2-iodo-3-(2,2,2-trifluoroethyl)benzo[b]thiophen-7-yl)-1-(tetrahydro-2H-pyran-4-yl)piperidin-4-amine (0.150 g, 286.05 μmol, 31.41% yield) as an light yellow oil. LCMS: m/z=525[M−1]⁻

### Step 4. (3-methoxy-4-((3-(7-((1-(tetrahydro-2H-pyran-4-yl)piperidin-4-yl)amino)-3-(2,2,2-trifluoroethyl)benzo[b]thiophen-2-yl)prop-2-yn-1-yl)amino)phenyl)dimethylphosphine oxide (154)

Into a 4 mL vial purged and maintained with nitrogen atmosphere was placed 4-((2-iodo-3-(2,2,2-trifluoroethyl)benzo[b]thiophen-7-yl)amino)-1-methylpiperidine-3-carbonitrile (0.051 g, 97.26 μmol), (3-methoxy-4-(prop-2-yn-1-ylamino)phenyl)dimethylphosphine oxide (0.040 g, 168.61 μmol), Pd(PPh₃)₂Cl₂ (0.009 g, 12.75 μmol), CuI (0.004 g, 21.00 μmol), TEA (0.017 g, 168.00 μmol), DMF (1 mL), and stirred for 1.5 h at 50° C. The reaction was quenched with water (2 mL), extracted with EA (2 mL×2). The combined organic layers were washed with water (2 mL) and brine (2 mL) successively, separated, then concentrated with vacuum. The residue was purified with prep-HPLC (Mobile Phase A: water (NH₄OH), Mobile Phase B: MeOH; Flow rate: 40 mL/min; Gradient: 45-75-81% B (2-30-39 min); 266 nm; RT: 33.63-36.50 min) to afford (3-methoxy-4-((3-(7-((1-(tetrahydro-2H-pyran-4-yl)piperidin-4-yl)amino)-3-(2,2,2-trifluoroethyl)benzo[b]thiophen-2-yl)prop-2-yn-1-yl)amino)phenyl)dimethylphosphine oxide (154) (0.009 g, 14.20 μmol, 14.60% yield) as white solid. LCMS: m/z=634 [M+1]⁺.

¹H NMR (400 MHz, DMSO-d₆) δ 7.28-7.11 (m, 4H), 6.84 (dd, J=8.1, 2.9 Hz, 1H), 6.66 (d, J=7.9 Hz, 1H), 6.07 (t, J=6.3 Hz, 1H), 5.29 (d, J=7.9 Hz, 1H), 4.33 (d, J=6.3 Hz, 2H), 3.93-3.73 (m, 7H), 3.27 (t, J=11.5 Hz, 2H), 2.89 (d, J=10.9 Hz, 2H), 2.41 (d, J=11.0 Hz, 2H), 2.22 (t, J=11.1 Hz, 2H), 1.91 (d, J=12.5 Hz, 2H), 1.67 (d, J=12.5 Hz, 2H), 1.58 (d, J=13.1 Hz, 6H), 1.52-1.37 (m, 4H).

### Example 155

### diethyl(3-methoxy-4-((3-(7-((1-(tetrahydro-2H-pyran-4-yl)piperidin-4-yl)amino)-3-(2,2,2-trifluoroethyl)benzo[b]thiophen-2-yl)prop-2-yn-1-yl)amino)phenyl)phosphine oxide (155)

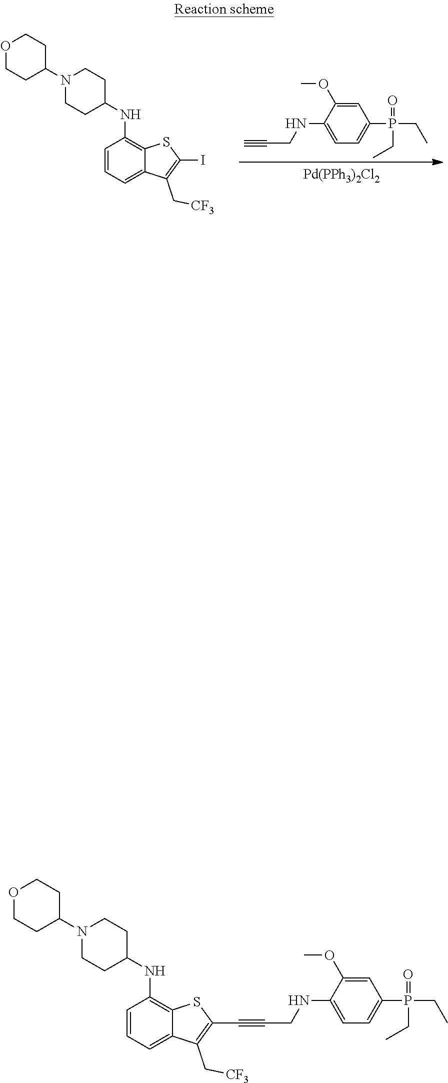 [SMILES: FC(F)(F)Cc1c(I)sc2c(NC3CCN(C4CCOCC4)CC3)cccc12 ; C#CCNc1ccc(P(=O)(CC)CC)cc1OC ; CCP(=O)(CC)c1ccc(NCC#Cc2sc3c(NC4CCN(C5CCOCC5)CC4)cccc3c2CC(F)(F)F)c(OC)c1 ; "Pd(PPh3)2Cl2"]

### Experiment Details

### Step 1. diethyl(3-methoxy-4-((3-(7-((1-(tetrahydro-2H-pyran-4-yl)piperidin-4-yl)amino)-3-(2,2,2-trifluoroethyl)benzo[b]thiophen-2-yl)prop-2-yn-1-yl)amino)phenyl)phosphine oxide (155)

Into a 4 mL vial purged and maintained with nitrogen atmosphere was placed 4-((2-iodo-3-(2,2,2-trifluoroethyl)benzo[b]thiophen-7-yl)amino)-1-methylpiperidine-3-carbonitrile (0.063 g, 120.14 μmol), diethyl(3-methoxy-4-(prop-2-yn-1-ylamino)phenyl)phosphine oxide (0.046 g, 173.40 μmol), Pd(PPh₃)₂Cl₂ (0.012 g, 17.00 μmol), CuI (0.010 g, 52.51 μmol), TEA (0.027 g, 266.83 μmol), DMF (0.5 mL), and stirred overnight at 50° C. The reaction was quenched with water (2 mL), extracted with EA (2 mL×2). The combined organic layers were washed with water (2 mL) and brine (2 mL) successively, separated, then concentrated with vacuum. The residue was purified with prep-HPLC (Mobile Phase A: water (NH₄OH), Mobile Phase B: ACN; F low rate: 70 mL/min; Gradient: 45-70-82% B (2-30-46 min); 266 nm; RT: 39.43-44.79 min) to afford diethyl(3-methoxy-4-((3-(7-((1-(tetrahydro-2H-pyran-4-yl)piperidin-4-yl)amino)-3-(2,2,2-trifluoroethyl)benzo[b]thiophen-2-yl)prop-2-yn-1-yl)amino)phenyl)phosphine oxide (155) (0.025 g, 37.78 μmol, 31.44% yield) as white sol id. LCMS: m/z=662 [M+1]⁺.

¹H NMR (400 MHz, DMSO-d₆) δ 7.29-7.05 (m, 4H), 6.91-6.82 (m, 1H), 6.66 (d, J=7.8 Hz, 1H), 6.08 (t, J=6.3 Hz, 1H), 5.29 (d, J=7.9 Hz, 1H), 4.34 (d, J=6.3 Hz, 2H), 3.98-3.69 (m, 7H), 3.27 (t, J=11.5 Hz, 2H), 2.89 (d, J=10.7 Hz, 2H), 2.44 (s, 2H), 2.23 (s, 2H), 2.00-1.74 (m, 6H), 1.67 (d, J=12.2 Hz, 2H), 1.58-1.33 (m, 4H), 1.02-0.79 (m, 6H).

### Example 156

### diethyl(4-((3-(7-(((3S,4R)-3-fluoro-1-(tetrahydro-2H-pyran-4-yl)piperidin-4-yl)amino)-3-(2,2,2-trifluoroethyl) benzo[b]thiophen-2-yl)prop-2-yn-1-yl)amino)-3-methoxyphenyl)phosphine oxide (156)

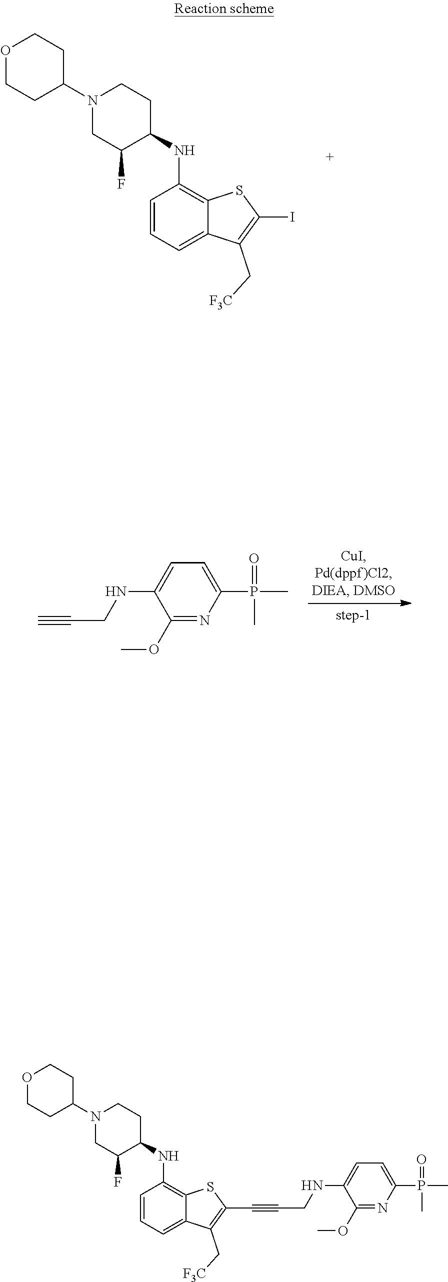 [SMILES: F[C@H]1CN(C2CCOCC2)CC[C@H]1Nc1cccc2c(CC(F)(F)F)c(I)sc12 ; C#CCNc1ccc(P(C)(C)=O)nc1OC ; COc1nc(P(C)(C)=O)ccc1NCC#Cc1sc2c(N[C@@H]3CCN(C4CCOCC4)C[C@@H]3F)cccc2c1CC(F)(F)F]

### Experiment Details

### Step 1. diethyl(4-((3-(7-(((3S,4R)-3-fluoro-1-(tetrahydro-2H-pyran-4-yl)piperidin-4-yl)amino)-3-(2,2,2-trifluoroethyl)benzo[b]thiophen-2-yl)prop-2-yn-1-yl)amino)-3-methoxyphenyl)phosphine oxide (156)

Into a 8-mL sealed tube purged and maintained with an inert atmosphere of nitrogen, was placed (3S,4R)-3-fluoro-N-(2-iodo-3-(2,2,2-trifluoroethyl)benzo[b]thiophen-7-yl)-1-(tetrahydro-2H-pyran-4-yl)piperidin-4-amine (0.109 g, 0.20 mmol), diethyl(3-methoxy-4-(prop-2-yn-1-ylamino)phenyl)phosphine oxide (0.112 g, 0.42 mmol), Pd(dppf)Cl₂ (0.021 g, 0.03 mmol), CuI (0.038 g, 0.20 mmol), DIEA (0.128 g, 0.99 mmol), DMSO (3 mL). The reaction mixture was stirred at 50° C. for 1 h. The mixture was purified by preparative HPLC (Mobile Phase A: water (10 mmoL/L ammonium hydroxide), Mobile Phase B: MeCN; Flow rate: 70 mL/mi n; Gradient: 40-70-70% B (2-32-60 min) 260 nm; RT: 31.40-33.96 min) to afford diethyl(4-((3-(7-(((3S,4R)-3-fluoro-1-(tetrahydro-2H-pyran-4-yl)piperidin-4-yl)amino)-3-(2,2,2-trifluoroethyl)benzo[b]thiophen-2-yl)prop-2-yn-1-yl)amino)-3-methoxyphenyl)phosphine oxide (156)(0.0674 g, 49.34% yield) as white solid. LCMS: m/z=680 [M+1]⁺

¹H NMR (400 MHz, CDCl₃) δ 7.60-7.43 (m, 1H), 7.37 (d, J=22.3 Hz, 2H), 7.24 (d, J=10.8 Hz, 1H), 7.13 (d, J=8.4 Hz, 1H), 6.86 (d, J=7.2 Hz, 1H), 6.72 (s, 1H), 4.96 (s, 1H), 4.36 (d, J=5.8 Hz, 2H), 4.18-4.01 (m, 2H), 3.99 (s, 1H), 3.95 (s, 3H), 3.88-3.74 (m, 1H), 3.66-3.52 (m, 2H), 3.48-3.36 (m, 3H), 3.34-3.11 (m, 2H), 2.27-1.79 (m, 8H), 1.76-1.41 (m, 4H), 1.26-1.03 (m, 6H).

### Example 157

### (4-((3-(7-(((3S,4R)-3-fluoro-1-(tetrahydrofuran-3-yl)piperidin-4-yl)amino)-3-(2,2,2-trifluoroethyl)benzo[b]thiophen-2-yl)prop-2-yn-1-yl)amino)-3-methoxyphenyl)dimethylphosphine oxide (157)

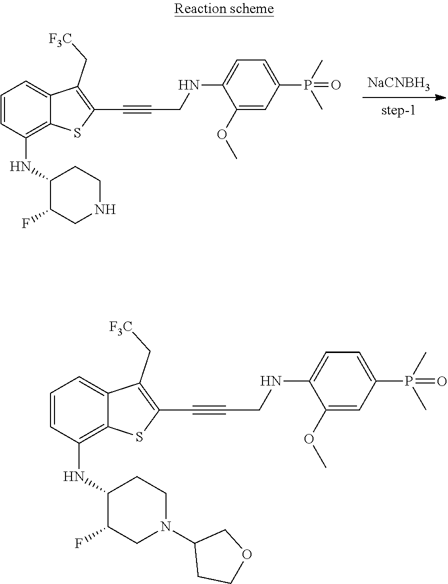

### Experimental Details

### Step 1. (4-((3-(7-(((3S,4R)-3-fluoro-1-(tetrahydrofuran-3-yl)piperidin-4-yl)amino)-3-(2,2,2-trifluoroethyl)benzo[b]thiophen-2-yl)prop-2-yn-1-yl)amino)-3-methoxyphenyl)dimethylphosphine oxide (157)

Into a 100-mL three necked bottle purged and maintained with an inert atmosphere of nitrogen, was placed (4-((3-(7-(((3S,4R)-3-fluoropiperidin-4-yl)amino)-3-(2,2,2-trifluoroethyl)benzo[b]thiophen-2-yl)prop-2-yn-1-yl)amino)-3-methoxyphenyl)dimethylphosphine oxide (0.052 g, 0.092 mmol), dihydrofuran-3(2H)-one (0.153 g, 1.78 mmol), NaCNBH₃ (0.132 g, 3.08 mmol), and MeOH (10 mL). The reaction mixture was stirred at 60° C. for 16 h. The resulting solution was added to water (30 mL). The resulting solution was extracted with EA (2×15 mL). The organic layers combined and washed with brine (20 mL), dried over anhydrous Na₂SO₄, the residue was concentrated under vacuum. The mixture was purified by preparative HPLC (Mobile Phase A: water (10 mmoL/L ammonium hydroxide), Mobile Phase B: ACN; Flow rate: 40 mL/min; Gradient: 45-75-75% B (2-30-60 min); 266 nm; RT: 29.14-31.27 min). This resulted in 0.060 g (86.57% yield) of (4-((3-(7-(((3S,4R)-3-fluoro-1-(tetrahydrofuran-3-yl)piperidin-4-yl)amino)-3-(2,2,2-trifluoroethyl)benzo[b]thio phen-2-yl)prop-2-yn-1-yl)amino)-3-methoxyphenyl)dimethylphosphine oxide (157) as off white solid. LCMS: m/z=638 [M+1]⁺.

¹H NMR (400 MHz, DMSO) δ 7.31-7.10 (m, 4H), 6.85 (dd, J=8.0, 2.8 Hz, 1H), 6.78 (d, J=7.7 Hz, 1H), 6.08 (t, J=6.3 Hz, 1H), 5.18 (d, J=8.4 Hz, 1H), 4.81 (dd, J=49.4, 7.6 Hz, 1H), 4.34 (d, J=6.3 Hz, 2H), 3.85 (s, 3H), 3.81-3.58 (m, 5H), 3.48 (dd, J=16.4, 9.0 Hz, 1H), 3.13 (t, J=10.9 Hz, 1H), 3.05-2.87 (m, 2H), 2.74 (d, J=11.1 Hz, 1H), 2.46-2.14 (m, 2H), 2.02-1.84 (m, 2H), 1.82-1.66 (m, 2H), 1.60 (s, 3H), 1.56 (s, 3H).

### Example 160

### 2-dimethylphosphoryl-5-[3-[7-[[(3S,4R)-3-fluoro-1-tetrahydropyran-4-yl-4-piperidyl]amino]-3-(2,2,2-trifluoro ethyl)benzothiophen-2-yl]prop-2-ynylamino]-4-methoxy-N-methyl-benzamide (160)

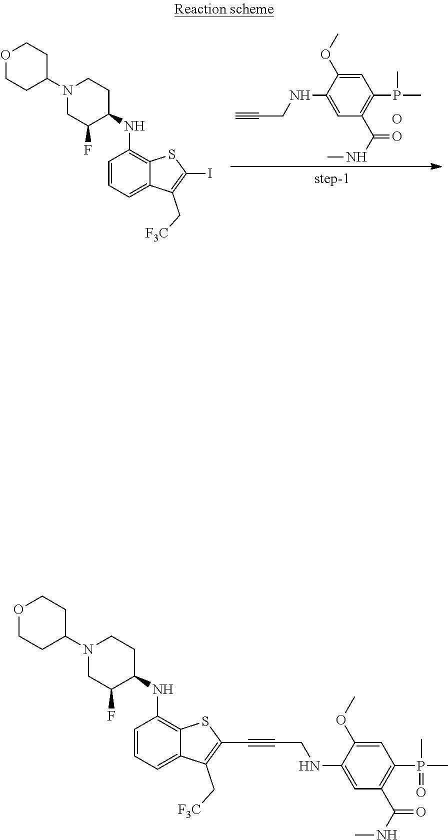

### Experiment Details

### Step 1. Synthesis of 2-dimethylphosphoryl-5-[3-[7-[[(3S,4R)-3-fluoro-1-tetrahydropyran-4-yl-4-piperidyl]amino]-3-(2,2,2-trifluoroethyl)benzothiophen-2-yl]prop-2-ynylamino]-4-methoxy-N-methyl-benzamide (160)

Into a 8-mL vial was placed (3S,4R)-3-fluoro-N-(2-iodo-3-(2,2,2-trifluoroethyl)benzo[b]thiophen-7-yl)-1-(tetrahydro-2H-pyran-4-yl)piperidin-4-amine (0.108 g, 199.13 μmol), 2-(dimethylphosphoryl)-4-methoxy-N-methyl-5-(prop-2-yn-1-ylamino)benzamide (0.105 g, 356.80 μmol), bis(triphenylphosphine)palladium(II) chloride (0.022 g, 31.16 μmol), copper(I) iodide (0.015 g, 78.76 μmol), N,N-Diisopropylethylamine (0.096 g, 742.79 μmol), methyl sulfoxide (10 mL). The mixture was stirred at 60° C. for 1 h. The resulted reaction was diluted with water (20 mL) and extracted with EA (10 mL×3). The organic phase was dried over Na₂SO₄ and concentrated under vacuum. The resulted reaction was purified by C₁₈ chromatography column eluted with A CN/water (0.1% ammonium bicarbonate) (v/v=1/2) to give the product. This resulted in 10.7 mg (7.58% yield) of 2-dimethylphosphoryl-5-[3-[7-[[(3S,4R)-3-fluoro-1-tetrahydropyran-4-yl-4-piperidyl]amino]-3-(2,2,2-tri fluoroethyl)benzothiophen-2-yl]prop-2-ynylamino]-4-methoxy-N-methyl-benzamide (160) as white solid. LCMS: m/z=709 [M+1]⁺.

¹H NMR (400 MHz, DMSO-d6) δ 8.42 (d, J=4.7 Hz, 1H), 7.46 (d, J=12.8 Hz, 1H), 7.25 (dt, J=15.0, 7.9 Hz, 3H), 6.96 (d, J=4.3 Hz, 1H), 6.79 (d, J=7.5 Hz, 1H), 6.23 (t, J=6.2 Hz, 1H), 5.11 (d, J=8.6 Hz, 1H), 4.87 (s, 1H), 4.75 (s, 1H), 4.37 (d, J=6.2 Hz, 3H), 3.90 (s, 1H), 3.86 (s, 4H), 3.82 (d, J=11.0 Hz, 2H), 3.27 (dd, J=12.9, 10.2 Hz, 3H), 3.13 (t, J=11.4 Hz, 1H), 2.90 (d, J=11.3 Hz, 2H), 2.75 (d, J=4.5 Hz, 4H), 2.44 (d, J=12.6 Hz, 1H), 2.30 (d, J=11.1 Hz, 1H), 1.87 (q, J=11.0 Hz, 1H), 1.75 (d, J=12.2 Hz, 1H), 1.66 (d, J=13.9 Hz, 6H), 1.66 (s, 2H), 1.49-1.36 (m, 2H).

### Example 161

### (3-cyclopropoxy-4-((3-(7-(((3S,4R)-3-fluoro-1-(tetrahydro-2H-pyran-4-yl)piperidin-4-yl)amino)-3-(2,2,2-trifluoroethyl)benzo[b]thiophen-2-yl)prop-2-yn-1-yl)amino)phenyl)dimethylphosphine oxide (161)

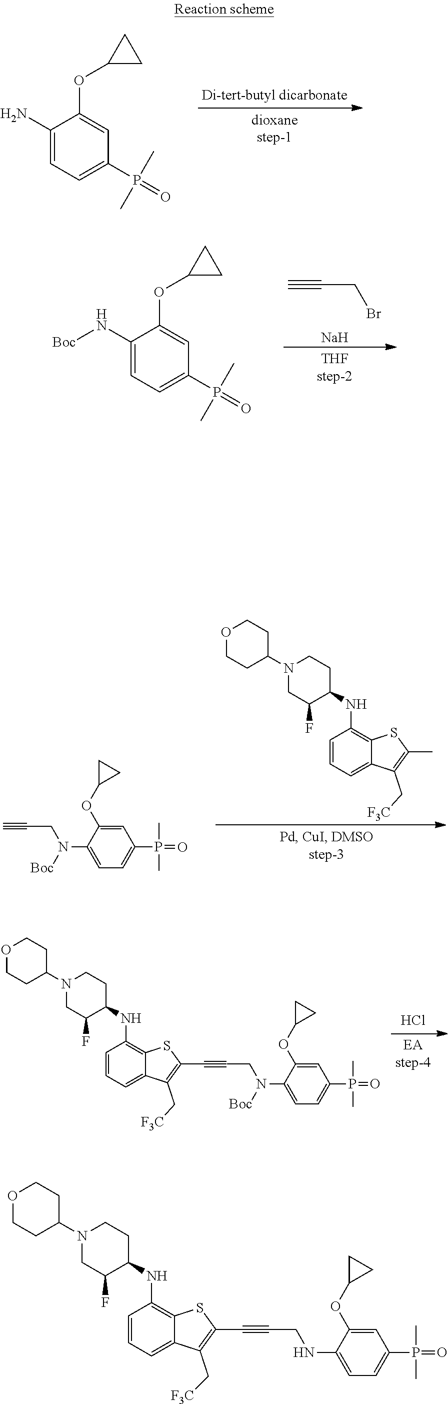

### Experiment Details

### Step 1 tert-butyl (2-cyclopropoxy-4-(dimethylphosphoryl)phenyl)carbamate

Into a 25 ml 3-necked flask was placed (4-amino-3-cyclopropoxyphenyl)dimethylphosphine oxide (2.67 g, 11.85 mmol), Di-tert-butyl dicarbonate (11.26 g, 51.59 mmol), dioxane (30 mL). The reaction mixture was stirred under nitrogen at 100° C. for 5 h. The crude product was purified with silica gel column eluted with methanol/dichloromethane (v/v=1/9). This resulted in 2.32 g, (60.15% yield) of tert-butyl (2-cyclopropoxy-4-(dimethylphosphoryl)phenyl)carbamate as white solid. LCMS: m/z=326 [M+1]⁺.

### Step 2. tert-butyl (2-cyclopropoxy-4-(dimethylphosphoryl)phenyl)(prop-2-yn-1-yl)carbamate

Into a 25-mL three necked bottle purged and maintained with an inert atmosphere of nitrogen, was placed tert-butyl (2-cyclopropoxy-4-(dimethylphosphoryl)phenyl)carbamate (0.231 g, 710.02 umol) and THF (3 mL). The reaction mixture was stirred at 0° C. Then NaH (0.117 g, 4.87 mmol) was added. The mixture was stirred at 0° C. for 1 h. 3-bromoprop-1-yne (0.594 g, 4.99 mmol) was added to the reaction at 0° C. The reaction mixture was stirred at 70° C. for 3 h. The resulting solution was added to MeOH (1 mL). The reaction mixture was filtered through celite pad and celite pad was washed with ACN (3×20 mL). The filterate w as then concentrated under reduced pressure to afford the crude product. The crude product was purified with silica gel column eluted with methanol/dichloromethane (v/v=1/9). This resulted in 0.104 g, (40.30% yield) of tert-butyl (2-cyclopropoxy-4-(dimethylphosphoryl)phenyl)(prop-2-yn-1-yl)carbamate as yellow solid. LCMS: m/z=364 [M+1]⁺.

### Step 3 tert-butyl (2-cyclopropoxy-4-(dimethylphosphoryl)phenyl)(3-(7-(((3S,4R)-3-fluoro-1-(tetrahydro-2H-pyran-4-yl)piperidin-4-yl)amino)-3-(2,2,2-trifluoroethyl)benzo[b]thiophen-2-yl)prop-2-yn-1-yl)carbamate

Into a 10-mL round-bottom flask was placed (3S,4R)-3-fluoro-N-(2-iodo-3-(2,2,2-trifluoroethyl)benzo[b]thiophen-7-yl)-1-(tetrahydro-2H-pyran-4-yl)piperidin-4-amine (0.094 g, 197.31 umol), tert-butyl (2-cyclopropoxy-4-(dimethylphosphoryl)phenyl)(prop-2-yn-1-yl)carbamate (0.098 g, 269.68 umol), Pd(PPh₃)₂Cl₂ (0.034 g, 48.16 μmol), CuI (0.014 g, 73.51 umol), DIEA (0.084 g, 649.94 umol), methyl sulfoxide (2 mL). The reaction was stirred under nitrogen atmosphere at RT for 4 h. The reaction mixture was filtered through celite pad and celite pad was washed with ACN (3×20 mL). The filterate was then concentrated under reduced pressure to afford the crude product. The crude product was purified by C18 column eluted with ACN/H₂O (v/v=2/3), filtered and concentrated under vacuum. This resulted in 0.091 g (67.50% yield) of tert-butyl (2-cyclopropoxy-4-(dimethylphosphoryl)phenyl)(3-(7-(((3S,4R)-3-fluoro-1-(tetrahydro-2H-pyran-4-yl)piperidin-4-yl)amino)-3-(2,2,2-trifluoroethyl)benzo[b]thiophen-2-yl)prop-2-yn-1-yl)carbamate as yellow solid. LCMS: m/z=778 [M+1]⁺.

### Step 4 (3-cyclopropoxy-4-((3-(7-(((3S,4R)-3-fluoro-1-(tetrahydro-2H-pyran-4-yl)piperidin-4-yl)amino)-3-(2,2,2-trifluoroethyl)benzo[b]thiophen-2-yl)prop-2-yn-1-yl)amino)phenyl)dimethylphosphine oxide (161)

Into a 10-mL round-bottom flask purged and maintained with an inert atmosphere of nitrogen, was placed tert-butyl (2-cyclopropoxy-4-(dimethylphosphoryl)phenyl)(3-(7-(((3S,4R)-3-fluoro-1-(tetrahydro-2H-pyran-4-yl)piperidin-4-yl)amino)-3-(2,2,2-trifluoroethyl)benzo[b]thiophen-2-yl)prop-2-yn-1-yl)carbamate (0.087 g, 111.84 um ol), ethyl acetate (1 mL) and HCl in EA solution (1 mL, 4 M). The reaction mixture was stirred at room temperature for 1 h. The mixture was concentrated to afford the crude product. The crude product was purified by preparative HPLC (Mobile Phase A: water (0.1% ammonium hydroxide), Mobile Phase B: ACN; Flow rate: 70 mL/min; Gradient: 35-70-72% B (2-30-34 min); 266 nm; 31.69-33.41) to provide the desired product. This resulted in 52.1 mg (67.50% yield) of (3-cyclopropoxy-4-((3-(7-(((3S,4R)-3-fluoro-1-(tetrahydro-2H-pyran-4-yl)piperidin-4-yl)amino)-3-(2,2,2-trifluoroethyl)benzo[b]thiophen-2-yl)prop-2-yn-1-yl)amino)phenyl)di methylphosphine oxide (161) as white solid. LCMS: m/z=678 [M+1]⁺.

¹H NMR (400 MHz, DMSO-d₆) δ 7.43-7.41 (m, 1H), 7.31-7.14 (m, 3H), 6.88-6.75 (m, 2H), 5.95 (t, J=6.3 Hz, 1H), 5.14 (d, J=8.5 Hz, 1H), 4.81 (d, J=49.6 Hz, 1H), 4.32 (d, J=6.3 Hz, 2H), 3.98-3.61 (m, 6H), 3.27 (t, J=11.6 Hz, 2H), 3.13 (t, J=11.2 Hz, 1H), 2.90 (d, J=11.0 Hz, 1H), 2.54 (s, 1H), 2.49-2.41 (m, 1H), 2.31 (t, J=11.2 Hz, 1H), 1.94-1.84 (m, 1H), 1.74 (d, J=13.3 Hz, 1H), 1.60 (t, J=12.1 Hz, 8H), 1.44 (td, J=12.0, 4.3 Hz, 2H), 0.86-0.68 (m, 4H).

### Example 162

### (2-fluoro-5-((3-(7-((3-fluoro-1-methylpiperidin-4-yl)amino)-3-(2,2,2-trifluoroethyl)benzo[b]thiophen-2-yl)prop-2-yn-1-yl)amino)-4-methoxyphenyl)dimethylphosphine oxide (162)

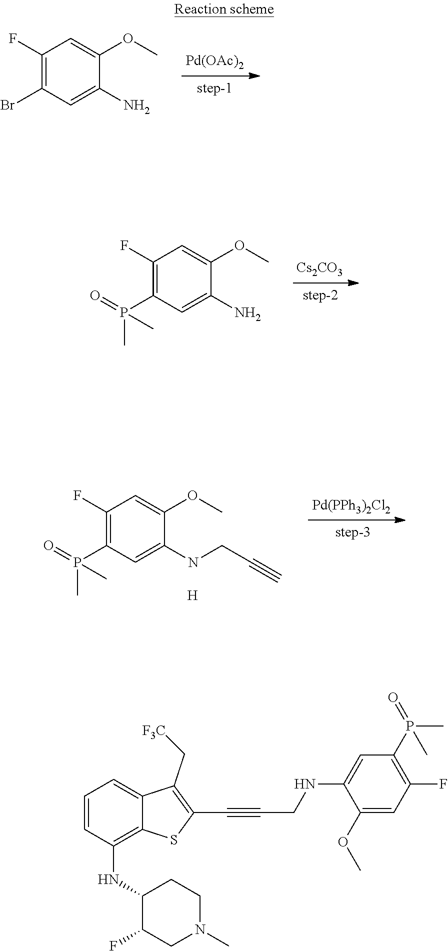

### Step 1. (5-amino-2-fluoro-4-methoxyphenyl)dimethylphosphine oxide

Into a 100-mL three necked bottle purged and maintained with an inert atmosphere of nitrogen, was placed 5-bromo-4-fluoro-2-methoxyaniline (3.00 g, 13.63 mmol), Pd(OAc)₂ (1.45 g, 6.46 mmol), Xantphos (1.32 g, 2.28 mmol), DIEA (2.15 g, 16.63 mmol) and DMF (20 mL). The reaction mixture was stirred at 130° C. for 0.5 h. Then was added Dimethylphosphine oxide (1.55 g, 19.86 mmol). The reaction mixture was stirred at 130° C. for another 1 h. LCMS showed that the reaction was completed. The reaction mixture was purified by C18 column eluted with ACN/H₂O (v/v=2/8). This resulted in 2.06 g (69.26% yield) of (5-amino-2-fluoro-4-methoxyphenyl)dimethylphosphine oxide as red oil. LCMS: m/z=218 [M+1]⁺.

### Step 2. (2-fluoro-4-methoxy-5-(prop-2-yn-1-ylamino)phenyl)dimethylphosphine oxide

Into a 50-mL flask was placed (5-amino-2-fluoro-4-methoxyphenyl)dimethylphosphine oxide (0.606 g, 2.79 mmol), 3-Bromopropyne (0.392 g, 3.29 mmol), Cs₂CO₃ (0.999 g, 3.07 mmol), KI (0.225 g, 1.36 mmol), DMF (5 mL). The reaction mixture was stirred at 80° C. for 4 h. LCMS showed that the reaction was completed. The reaction mixture was purified by C18 column eluted with ACN/H₂O (v/v=2/8). This resulted in 0.075 g (10.53% yield) of (2-fluoro-4-methoxy-5-(prop-2-yn-1-ylamino)phenyl)dimethylphosphine oxide as red solid. LCMS: m/z=256 [M+1]⁺.

### Step 3. (2-fluoro-5-((3-(7-((3-fluoro-1-methylpiperidin-4-yl)amino)-3-(2,2,2-trifluoroethyl)benzo[b]thiophen-2-yl)prop-2-yn-1-yl)amino)-4-methoxyphenyl)dimethylphosphine oxide (162)

Into a 50-mL three necked bottle and maintained with an inert atmosphere of nitrogen, was placed (3S,4R)-3-fluoro-N-(2-iodo-3-(2,2,2-trifluoroethyl)benzo[b]thiophen-7-yl)-1-methylpiperidin-4-amine (0.093 g, 0.19 m mol), (2-fluoro-4-methoxy-5-(prop-2-yn-1-ylamino)phenyl)dimethylphosphine oxide (0.078 g, 0.31 mmol), CuI (0.034 g, 0.18 mmol), Pd(PPh₃)₂Cl₂ (0.066 g, 0.93 mmol), DIEA (0.079 g, 0.61 mmol) and DMSO (5 mL). The reaction mixture was stirred at room temperature for 1 h. LCMS showed that the reaction was completed. The mixture was purified by preparative HPLC (Mobile Phase A: water (10 mmoL/L ammonium hydroxide), Mobile Phase B: ACN; Flow rate: 40 mL/min; Gradient: 30-50-50% B (2-30-60 min); 260 nm; R T: 25.38-30.64 min). This resulted in 0.019 g (16.09% yield) of (2-fluoro-5-((3-(7-((3-fluoro-1-methylpiperidin-4-yl)amino)-3-(2,2,2-trifluoroethyl)benzo[b]thiophen-2-yl)prop-2-yn-1-yl)amino)-4-methoxyphenyl)dimethylphosphine oxide (162) as off white solid. LCMS: m/z=600 [M+1]⁺.

¹H NMR (400 MHz, DMSO) δ 7.31-7.16 (m, 2H), 7.03 (dd, J=12.6, 6.4 Hz, 1H), 6.93 (dd, J=11.2, 4.5 Hz, 1H), 6.78 (d, J=7.6 Hz, 1H), 5.67 (t, J=6.4 Hz, 1H), 5.15 (d, J=8.5 Hz, 1H), 4.79 (d, J=49.9 Hz, 1H), 4.29 (d, J=6.4 Hz, 2H), 3.86 (s, 3H), 3.82-3.73 (m, 2H), 3.72-3.57 (m, 1H), 3.03 (t, J=10.9 Hz, 1H), 2.79 (d, J=10.4 Hz, 1H), 2.26 (d, J=13.0 Hz, 1H), 2.17 (d, J=9.4 Hz, 3H), 2.08 (t, J=11.3 Hz, 1H), 2.01-1.87 (m, 1H), 1.72 (d, J=11.0 Hz, 1H), 1.66 (s, 3H), 1.63 (s, 3H).

### Example 163

### (7-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)benzo[d][1,3]dioxol-4-yl)dimethylphosphine oxide (163)

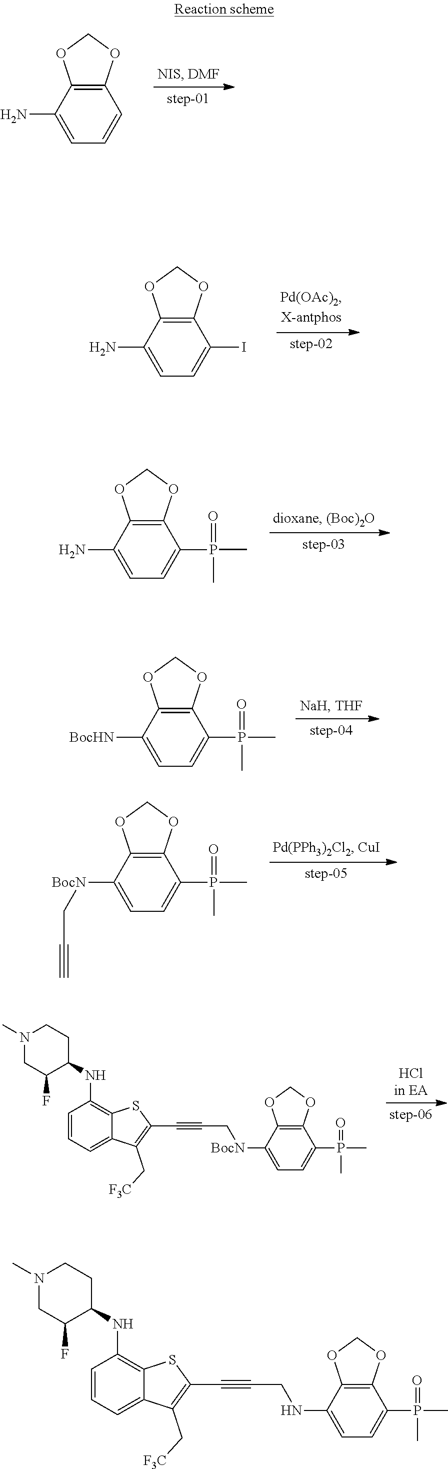

### Experimental Details

### Step 1. 7-iodobenzo[d][1,3]dioxol-4-amine

Into a 100-mL three necked bottle purged and maintained with an inert atmosphere of nitrogen, was placed benzo[d][1,3]dioxol-4-amine (2.000 g, 14.58 mmol), N,N-dimethylformamide (20 mL). The reaction mixture was cooled to −40° C. Then N-iodosuccinimide (3.604 g, 16.02 mmol) was added and stirred at the same temperature for 15 h. LCMS showed that the reaction was completed. The resulting solution was quenched with sodium bisulfite aqueous solution (10 mL) and extracted with EA (2×50 mL). The organic layer combined and washed with brine (50 mL), dried over anhydrous Na₂SO₄, the residue was concentrated under vacuum. The residues were purified by C₁₈ chromatography column eluted with ACN/H₂O (v/v=2/5). This resulted in 3.59 g (93.59% yield) of 7-iodobenzo[d][1,3]dioxol-4-amine as brown solid. LCMS: m/z=264 [M+1]⁺.

### Step 2. (7-aminobenzo[d][1,3]dioxol-4-yl)dimethylphosphine oxide

Into a 100-mL three necked bottle purged and maintained with an inert atmosphere of nitrogen, was placed 7-iodobenzo[d][1,3]dioxol-4-amine (3.58 g, 13.61 mmol), palladium (II) acetate (0.49 g, 2.18 mmol), dimethylbisdiphenylphosphinoxanthene (1.17 g, 2.02 mmol), N,N-diisopropylethylamine (3.57 g, 27.62 mmol), N, N-dimethylformamide (20 mL). The reaction mixture was heated to 80° C. Then methylphosphinoylmethane (4.01 g, 51.38 mmol) was added and stirred at the same temperature for 1.0 h. LCMS showed that the reaction was completed. The residue was filtered and purified by C₁₈ chromatography column eluted with AC N/H₂O (0.05% NH₄HCO₃) (v/v=1/10). This resulted in 2.71 g (93.03% yield) of (7-aminobenzo[d][1,3]dioxol-4-yl)dimethylphosphine oxide as brown solid. LCMS: m/z=701 [M+1]⁺.

### Step 3. tert-butyl (7-(dimethylphosphoryl)benzo[d][1,3]dioxol-4-yl)carbamate

Into a 100-mL three necked bottle purged and maintained with an inert atmosphere of nitrogen, was placed (7-aminobenzo[d][1,3]dioxol-4-yl)dimethylphosphine oxide (0.64 g, 3.00 mmol), di-tert-butyl dicarbonate (1.09 g, 4.99 mmol), 1,4-dioxane (20 mL). The reaction mixture was stirred at 110° C. for 17 h. LCMS showed that the reaction was completed. The resulting solution was concentrated and purified by C₁₈ chromatography column eluted with ACN/H₂O (v/v=1/1). This resulted in 0.82 g (87.18% yield) of tert-butyl (7-(dimethylphosphoryl)benzo[d][1,3]dioxol-4-yl)carbamate as off-white solid. LCMS: m/z=314 [M+1]⁺.

### Step 4. tert-butyl (7-(dimethylphosphoryl)benzo[d][1,3]dioxol-4-yl)(prop-2-yn-1-yl)carbamate

Into a 100-mL three necked bottle purged and maintained with an inert atmosphere of nitrogen, was placed tert-butyl (7-(dimethylphosphoryl)benzo[d][1,3]dioxol-4-yl)carbamate (0.81 g, 2.59 mmol), tetrahydrofuran (10 mL). The reaction mixture was stirred at 0° C. Then sodium hydride (0.51 g, 21.25 mmol) was added. The mixture was stirred at 20° C. for 1.0 h. Then 3-bromopropyne (2.937 g, 24.69 mmol) was added to the reaction at −10° C. The reaction mixture was stirred at 80° C. for 16 h. The resulting solution was added to water (10 mL). The resulting solution was extracted with EA (2×50 mL). The organic layer combined and washed with brine (50 mL), dried over anhydrous Na₂SO₄, the residue was concentrated under vacuum. The residue was purified by C₁₈ chromatography column eluted with ACN/H₂O (v/v=1/1). This resulted in 0.60 g (66.06% yield) of tert-butyl (7-(dimethylphosphoryl)benzo[d][1,3]dioxol-4-yl)(prop-2-yn-1-yl)carba mate as yellow oil. LCMS: m/z=352 [M+1]⁺.

### Step 5. tert-butyl (7-(dimethylphosphoryl)benzo[d][1,3]dioxol-4-yl)(3-(7-(((3S,4R)-3-fluoro-1-methylpiperidin-4-yl)amino)-3-(2,2,2-trifluoroethyl)benzo[b]thiophen-2-yl)prop-2-yn-1-yl)carbamate

Into a 50-mL three necked bottle purged and maintained with an inert atmosphere of nitrogen, was placed [1,1′-bis(diphenylphosphino)ferrocene]dichloropalladium(II) (0.074 g, 104.83 μmol), cuprous iodide (0.013 g, 68.26 μmol), triethylamine (0.320 g, 3.16 mmol), (3S,4R)-3-fluoro-N-(2-iodo-3-(2,2,2-trifluoroethyl)benzo[b]thiophen-7-yl)-1-methylpiperidin-4-amine (0.301 g, 637.33 μmol), methyl sulfoxide (20 mL). The reaction mixture was stirred at 80 refor 10 min. Then tert-butyl (7-(dimethylphosphoryl)benzo[d][1,3]dioxol-4-yl)(prop-2-yn-1-yl)carbamate (0.292 g, 831.12 μmol) was added and stirred at the same temperature for 1 h. LCMS showed that the reaction was completed. The resulting solution was quenched with water (200 mL) and extracted with EA (2×100 mL). The organic layer was combined and washed with brine (100 mL), dried over anhydrous Na₂SO₄, the residue was concentrated under vacuum. The residue was purified by C₁₈ chromatography column eluted with ACN/H₂O (v/v=2/5). This resulted in 0.33 g (74.42% yield) of tert-butyl (7-(dimethyl phosphoryl)benzo[d][1,3]dioxol-4-yl)(3-(7-(((3S,4R)-3-fluoro-1-methylpiperidin-4-yl)amino)-3-(2,2,2-trifluoroethyl) benzo[b]thiophen-2-yl)prop-2-yn-1-yl)carbamate as brown solid. LCMS: m/z=696 [M+1]⁺.

### Step 6. (7-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)benzo[d][1,3]dioxol-4-yl)dimethylphosphine oxide (163)

Into a 20-mL round-bottom flask purged and maintained with an inert atmosphere of nitrogen, was placed tert-butyl (7-(dimethylphosphoryl)benzo[d][1,3]dioxol-4-yl)(3-(7-(((3S,4R)-3-fluoro-1-methylpiperidin-4-yl)amino)-3-(2,2,2-trifluoroethyl)benzo[b]thiophen-2-yl)prop-2-yn-1-yl)carbamate (0.32 g, 459.97 μmol), ethyl acetate (5 mL) and HCl in EA solution (5 mL, 4 M). The reaction mixture was stirred at room temperature for 0.5 h. LCMS showed that the reaction was completed. Then saturated sodium hydrogen carbonate aqueous solution was added to the mixture until pH=7-8. The resulting solution was extracted with EA (2×40 mL). The organic layer was combined, washed with brine (40 mL), dried over anhydrous Na₂SO₄ and concentrated under vacuum. The mixture was purified by preparative HPLC (Mobile Phase A: water (10 mmoL/L ammonium hydroxide), Mobile Phase B: ACN; Flow rate: 70 mL/min; Gradient: 35-66-66% B (2-30-33 min; 262 nm; RT: 29.31-32.14 min). This resulted in 0.170 g (62.06% yield) of (7-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)benzo[d][1,3]dioxol-4-yl)dimethylphosphine oxide (163) as yellow solid. LCMS: m/z=596 [M+1]⁺.

¹H NMR (400 MHz, DMSO) δ 7.27 (t, J=7.8 Hz, 1H), 7.21 (d, J=7.9 Hz, 1H), 7.07-7.01 (m, 1H), 6.78 (d, J=7.7 Hz, 1H), 6.60 (d, J=8.5 Hz, 1H), 6.34 (t, J=6.4 Hz, 1H), 6.05 (s, 2H), 5.20 (d, J=8.5 Hz, 1H), 4.86-4.69 (m, 1H), 4.33 (d, J=6.5 Hz, 2H), 3.88-3.74 (m, 2H), 3.73-3.56 (m, 1H), 3.07-2.98 (m, 1H), 2.80 (d, J=10.9 Hz, 1H), 2.34-2.24 (m, 1H), 2.17 (d, J=9.5 Hz, 3H), 2.08 (t, J=10.8 Hz, 1H), 2.00-1.89 (m, 1H), 1.76-1.67 (m, 1H), 1.60 (s, 3H), 1.57 (s, 3H).

### Example 164

### (2,2-difluoro-7-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)benzo[d][1,3]dioxol-4-yl)dimethylphosphine oxide (164)

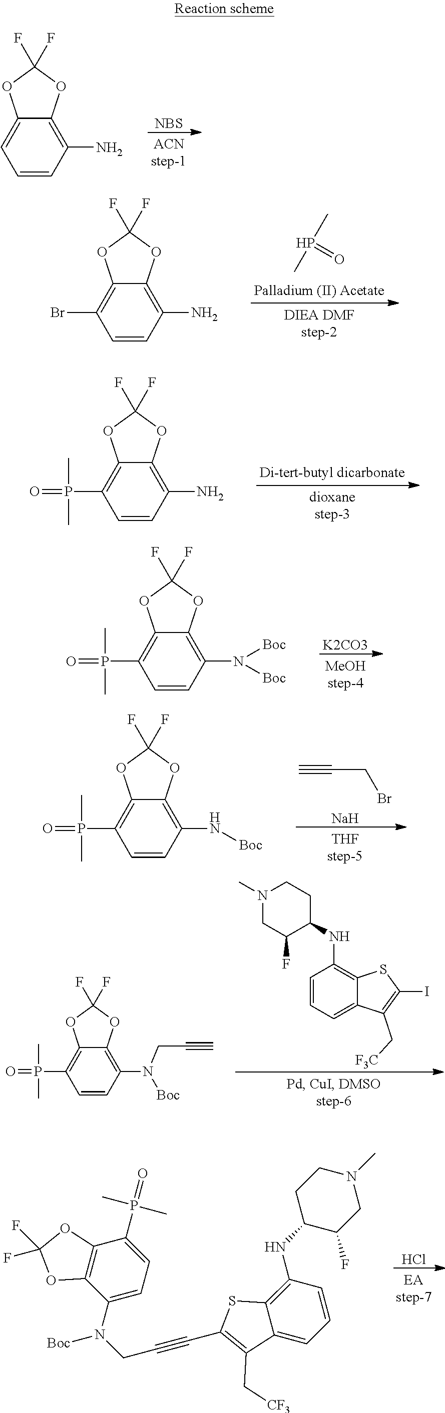

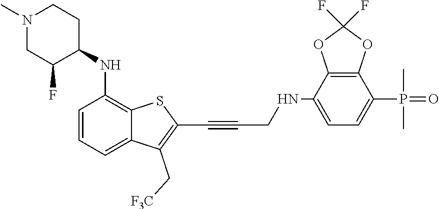 [SMILES: CN1CC[C@@H](Nc2cccc3c(CC(F)(F)F)c(C#CCNc4ccc(P(C)(C)=O)c5c4OC(F)(F)O5)sc23)[C@@H](F)C1]

### Experiment Details

### Step 1 7-bromo-2,2-difluorobenzo[d][1,3]dioxol-4-amine

Into a 50 ml 3-necked flask was placed 2,2-difluorobenzo[d][1,3]dioxol-4-amine (1.086 g, 6.27 mmol), NBS (1.189 g, 6.68 mmol), ACN (10 mL). The reaction was stirred at rt for 2 h. The reaction mixture was concentrated under reduced pressure to afford crude product. The resulting solution was added to water (20 mL). The resulting solution was extracted with EA (3×50 mL). The organic layers combined and washed with brine (50 mL), dried over anhydrous Na₂SO₄, the residue was concentrated under vacuum. The crude product was purified with silica gel column eluted with EA/hexane (v/v=1/4). This resulted in 1.21 g, (76.53% yield) of 7-bromo-2,2-difluorobenzo[d][1,3]dioxol-4-amine as yellow solid. LCMS: m/z=252 [M+1]⁺.

### Step 2 (7-amino-2,2-difluorobenzo[d][1,3]dioxol-4-yl)dimethylphosphine oxide

Into a 25 ml 3-necked flask was placed 7-bromo-2,2-difluorobenzo[d][1,3]dioxol-4-amine (1.23 g, 4.88 mmol), Palladium (II) Acetate (0.238 g, 1.06 mmol), Xantphos (0.529 g, 914.24 umol), DIEA (1.93 g, 14.93 m mol), DMF (10 mL) The reaction was stirred under nitrogen atmosphere at 130° C. for 0.5 h. Then dimethyl phosphine oxide (1.053 g, 13.49 mmol) was added at 130° C. The reaction was stirred under nitrogen atmosphere at 130° C. for 1 h. The reaction mixture was filtered through celite pad and celite pad was washed with ACN (3×20 mL). The filterate was then concentrated under reduced pressure to afford the crude product. The reaction mixture was purified by C18 column eluted with ACN/H₂O (v/v=1/9), filtered and concentrated under vacuum. This resulted in 1.173 g, (96.46% yield) of (7-amino-2,2-difluorobenzo[d][1,3]dioxol-4-yl)dimethylphosphine oxide as yellow solid. LCMS: m/z=250 [M+1]⁺.

### Step 3 tert-butyl N-tert-butoxycarbonyl-N-(7-dimethylphosphoryl-2,2-difluoro-1,3-benzodioxol-4-yl)carba mate

Into a 25 ml 3-necked flask was placed (7-amino-2,2-difluorobenzo[d][1,3]dioxol-4-yl)dimethylphosphine oxide (0.450 g, 1.80 mmol), Di-tert-butyl dicarbonate (2.34 g, 10.72 mmol), dioxane (5 mL). The reaction mixture was stirred under nitrogen at 100° C. for 16 h. The crude product was purified with silica gel column eluted with methanol/dichloromethane (v/v=1/9). This resulted in 0.460 g, (56.67% yield) of tert-butyl N-tert-butoxycarbonyl-N-(7-dimethylphosphoryl-2,2-difluoro-1,3-benzodioxol-4-yl)carbamate as yellow oil. LCMS: m/z=450 [M+1]⁺.

### Step 4 tert-butyl (7-(dimethylphosphoryl)-2,2-difluorobenzo[d][1,3]dioxol-4-yl)carbamate

Into a 25 ml 3-necked flask was placed tert-butyl N-tert-butoxycarbonyl-N-(7-dimethylphosphoryl-2,2-difluoro-1,3-benzodioxol-4-yl)carbamate (0.449 g, 999.14 umol), K₂CO₃ (0.634 g, 4.58 mmol), MeOH (5 mL). The reaction mixture was stirred under nitrogen at 60° C. for 2 h. The reaction mixture was filtered through c elite pad and celite pad was washed with ACN (3×20 mL). The filterate was then concentrated under reduce d pressure to afford the crude product. The crude product was purified with silica gel column eluted with methanol/dichloromethane (v/v=1/8). This resulted in 0.288 g, (82.52% yield) of tert-butyl (7-(dimethylphosphoryl)-2,2-difluorobenzo[d][1,3]dioxol-4-yl)carbamate as yellow oil. LCMS: m/z=350 [M+1]⁺.

### Step 5. tert-butyl (7-(dimethylphosphoryl)-2,2-difluorobenzo[d][1,3]dioxol-4-yl)(prop-2-yn-1-yl)carbamate

Into a 25-mL three necked bottle purged and maintained with an inert atmosphere of nitrogen, was placed tert-butyl (7-(dimethylphosphoryl)-2,2-difluorobenzo[d][1,3]dioxol-4-yl)carbamate (0.159 g, 455.24 umol) and THF (3 mL). The reaction mixture was stirred at 0° C. Then NaH (0.052 g, 2.16 mmol) was added. The mixture was stirred at 0° C. for 1 h. 3-bromoprop-1-yne (0.281 g, 2.36 mmol) was added to the reaction at 0° C. The reaction mixture was stirred at room temperature for 4 h. The resulting solution was added to MeOH (1 mL). The reaction mixture was filtered through celite pad and celite pad was washed with ACN (3×20 mL). The filterate was then concentrated under reduced pressure to afford the crude product. The crude product was purified with silica gel column eluted with methanol/dichloromethane (v/v=1/9). This resulted in 0.126 g, (71.46% yield) of tert-butyl (7-(dimethylphosphoryl)-2,2-difluorobenzo[d][1,3]dioxol-4-yl)(prop-2-yn-1-yl)carbamate as yellow oil. LCMS: m/z=388 [M+1]⁺.

### Step 6 tert-butyl (7-(dimethylphosphoryl)-2,2-difluorobenzo[d][1,3]dioxol-4-yl)(3-(7-(((3S,4R)-3-fluoro-1-methylpiperidin-4-yl)amino)-3-(2,2,2-trifluoroethyl)benzo[b]thiophen-2-yl)prop-2-yn-1-yl)carbamate

Into a 10-mL round-bottom flask was placed (3S,4R)-3-fluoro-N-(2-iodo-3-(2,2,2-trifluoroethyl)benzo[b]thiophen-7-yl)-1-methylpiperidin-4-amine (0.102 g, 215.97 umol), tert-butyl (7-(dimethylphosphoryl)-2,2-difluorobenzo[d][1,3]dioxol-4-yl)(prop-2-yn-1-yl)carbamate (0.122 g, 314.98 umol), Pd(PPh₃)₂Cl₂ (0.034 g, 48.16 μmol), CuI (0.030 g, 157.52 umol), DIEA (0.093 g, 719.57 umol), methyl sulfoxide (2 mL). The reaction was stir red under nitrogen atmosphere at RT for 16 h. The reaction mixture was filtered through celite pad and celite pad was washed with ACN (3×20 mL). The filterate was then concentrated under reduced pressure to afford the crude product. The crude product was purified by C18 column eluted with ACN/H₂O (v/v=2/3), filtered and concentrated under vacuum. This resulted in 0.89 g (31.66% yield) of tert-butyl (7-(dimethylphosphoryl)-2,2-difluorobenzo[d][1,3]dioxol-4-yl)(3-(7-(((3S,4R)-3-fluoro-1-methylpiperidin-4-yl)amino)-3-(2,2,2-trifluoroethyl)benzo[b]thiophen-2-yl)prop-2-yn-1-yl)carbamate as yellow solid. LCMS: m/z=732 [M+1]⁺.

### Step 7 (2,2-difluoro-7-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)benzo[d][1,3]dioxol-4-yl)dimethylphosphine oxide (164)

Into a 10-mL round-bottom flask purged and maintained with an inert atmosphere of nitrogen, was placed tert-butyl (7-(dimethylphosphoryl)-2,2-difluorobenzo[d][1,3]dioxol-4-yl)(3-(7-(((3S,4R)-3-fluoro-1-methylpiperidin-4-yl)amino)-3-(2,2,2-trifluoroethyl)benzo[b]thiophen-2-yl)prop-2-yn-1-yl)carbamate (0.108 g, 147.60 umol), ethyl acetate (2 mL) and HCl in EA solution (2 mL, 4 M). The reaction mixture was stirred at room temperature for 1 h. The mixture was concentrated to afford the crude product. The crude product was purified by preparative HPLC (Mobile Phase A: water (0.1% ammonium hydroxide), Mobile Phase B: ACN; Flow rate: 40 mL/min; Gradient: 25-55-57% B (2-30-33 min); 260 nm; 30.91-33.01) to provide the desired product. This resulted in 0.0564 g (60.50% yield) of (2,2-difluoro-7-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)benzo[d][1,3]dioxol-4-yl)dimethylphosphine oxide (164) as white solid. LCMS: m/z=632 [M+1]⁺.

¹H NMR (400 MHz, DMSO-d₆) δ 7.37-7.20 (m, 3H), 7.11 (t, J=6.3 Hz, 1H), 6.88 (d, J=8.6 Hz, 1H), 6.78 (d, J=7.7 Hz, 1H), 5.21 (d, J=8.5 Hz, 1H), 4.80 (d, J=49.5 Hz, 1H), 4.41 (d, J=6.3 Hz, 2H), 3.89-3.74 (m, 2H), 3.68 (s, 1H), 3.03 (t, J=11.3 Hz, 1H), 2.80 (d, J=11.0 Hz, 1H), 2.27 (d, J=13.0 Hz, 1H), 2.18 (s, 3H), 2.06 (d, J=11.0 Hz, 1H), 2.02-1.89 (m, 1H), 1.80 (s, 1H), 1.66 (d, J=13.5 Hz, 6H).

### Example 165

### 5-(dimethylphosphoryl)-2-((3-(7-(((3S,4R)-3-fluoro-1-methylpiperidin-4-yl)amino)-3-(2,2,2-trifluoroethyl)benzo[b]thiophen-2-yl)prop-2-yn-1-yl)oxy)benzamide (165)

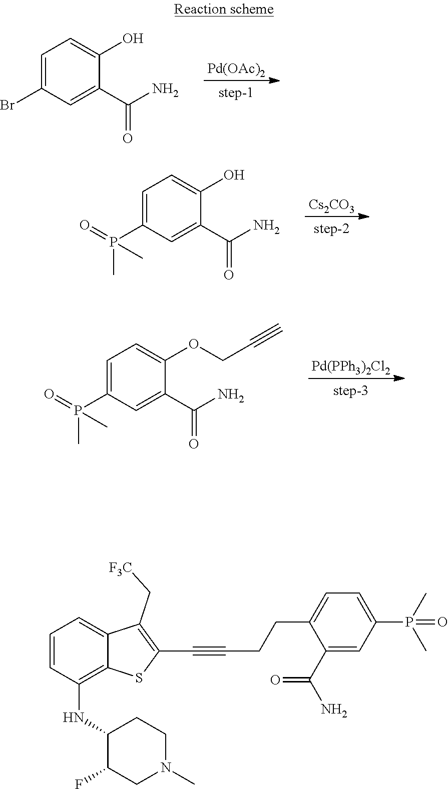

### Experimental Details

### Step 1. 5-(dimethylphosphoryl)-2-hydroxybenzamide

Into a 100-mL three necked bottle purged and maintained with an inert atmosphere of nitrogen, was placed 5-bromo-2-hydroxybenzamide (3.04 g, 14.07 mmol), Pd(OAc)₂ (1.34 g, 5.97 mmol), Xantphos (1.40 g, 2.42 mmol), DIEA (2.04 g, 15.78 mmol) and DMF (20 mL). The reaction mixture was stirred at 130° C. for 0.5 h. Then was added Dimethylphosphine oxide (2.53 g, 32.42 mmol). The reaction mixture was stirred at 130° C. for another 1 h. LCMS showed that the reaction was completed. The reaction mixture was purified by C18 column eluted with ACN/H₂O (v/v=2/8). This resulted in 2.38 g (79.34% yield) of 5-(dimethylphosphoryl)-2-hydroxybenzamide as red oil. LCMS: m/z=214 [M+1]⁺.

### Step 2. 5-(dimethylphosphoryl)-2-(prop-2-yn-1-yloxy)benzamide

Into a 50-mL flask was placed 5-(dimethylphosphoryl)-2-hydroxybenzamide (1.043 g, 4.75 mmol), 3-Bromo propyne (2.583 g, 21.71 mmol), DIEA (2.593 g, 20.06 mmol), DMF (5 mL). The reaction mixture was stirred at 80° C. for 4 h. LCMS showed that the reaction was completed. The reaction mixture was purified by C18 column eluted with ACN/H₂O (v/v=2/8). This resulted in 0.633 g (53.02% yield) of 5-(dimethylphosphoryl)-2-(prop-2-yn-1-yloxy)benzamide as red solid. LCMS: m/z=252 [M+1]⁺.

### Step 3. 5-(dimethylphosphoryl)-2-((3-(7-(((3S,4R)-3-fluoro-1-methylpiperidin-4-yl)amino)-3-(2,2,2-trifluoroethyl)benzo[b]thiophen-2-yl)prop-2-yn-1-yl)oxy)benzamide (165)

Into a 50-mL three necked bottle and maintained with an inert atmosphere of nitrogen, was placed (3S,4R)-3-fluoro-N-(2-iodo-3-(2,2,2-trifluoroethyl)benzo[b]thiophen-7-yl)-1-methylpiperidin-4-amine (0.105 g, 0.22 m mol), 5-(dimethylphosphoryl)-2-(prop-2-yn-1-yloxy)benzamide (0.177 g, 0.71 mmol), CuI (0.177 g, 0.93 mmol), Pd(PPh₃)₂Cl₂ (0.068 g, 0.93 mmol), DIEA (0.226 g, 1.75 mmol) and DMSO (5 mL). The reaction mixture was stirred at room temperature for 1 h. LCMS showed that the reaction was completed. The mixture was purified by preparative HPLC (Mobile Phase A: water (10 mmoL/L ammonium hydroxide), Mobile Phase B: ACN; Flow rate: 40 mL/min; Gradient: 30-50-100% B (2-30-60 min); 262 nm; RT: 20.583-22.420 min). This resulted in 0.055 g (41.53% yield) of 5-(dimethylphosphoryl)-2-((3-(7-(((3S,4R)-3-fluoro-1-methyl piperidin-4-yl)amino)-3-(2,2,2-trifluoroethyl)benzo[b]thiophen-2-yl)prop-2-yn-1-yl)oxy)benzamide (165) as off white solid. LCMS: m/z=596 [M+1]⁺.

¹H NMR (400 MHz, DMSO) δ 8.13 (dd, J=11.3, 1.7 Hz, 1H), 7.93-7.82 (m, 1H), 7.72 (d, J=8.6 Hz, 2H), 7.47 (d, J=7.8 Hz, 1H), 7.36-7.22 (m, 2H), 6.81 (d, J=7.4 Hz, 1H), 5.41 (s, 2H), 5.28 (d, J=8.5 Hz, 1H), 4.81 (d, J=49.7 Hz, 1H), 3.99-3.82 (m, 2H), 3.77-3.57 (m, 1H), 3.04 (t, J=10.7 Hz, 1H), 2.80 (d, J=10.5 Hz, 1H), 2.27 (d, J=12.8 Hz, 1H), 2.18 (s, 3H), 2.08 (t, J=11.3 Hz, 1H), 2.03-1.89 (m, 1H), 1.79-1.69 (m, 1H), 1.66 (s, 3H), 1.63 (s, 3H).

### Example 169&170

### (3-methoxy-4-((3-(7-(((Z)-1-methyl-3-(trifluoromethyl)piperidin-4-yl)amino)-3-(2,2,2-trifluoroethyl)benzo[b]thiophen-2-yl)prop-2-yn-1-yl)amino)phenyl)dimethylphosphine oxide (racemic) (169) and (3-methoxy-4-((3-(7-(((E)-1-methyl-3-(trifluoromethyl)piperidin-4-yl)amino)-3-(2,2,2-trifluoroethyl)benzo[b]thiophen-2-yl)prop-2-yn-1-yl)amino)phenyl)dimethylphosphine oxide (racemic) (170)

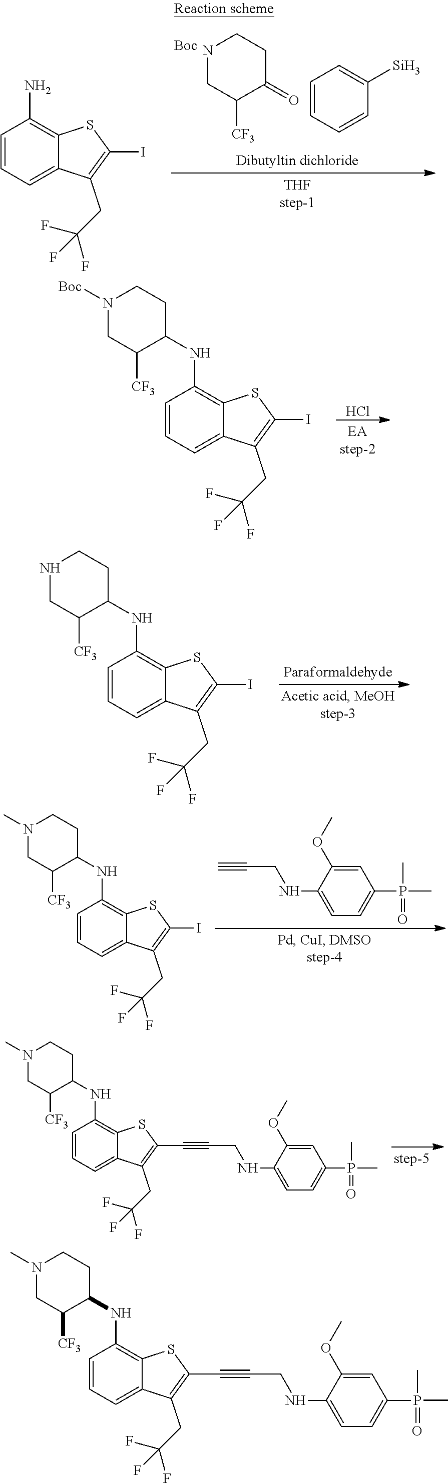

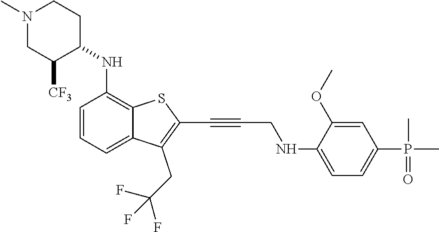

### Experimental Details

### Step 1. tert-butyl 4-((2-iodo-3-(2,2,2-trifluoroethyl)benzo[b]thiophen-7-yl)amino)-3-(trifluoromethyl)piperidine-1-carboxylate

Into a 50-mL three necked bottle purged and maintained with an inert atmosphere of nitrogen, was placed 2-iodo-3-(2,2,2-trifluoroethyl)benzo[b]thiophen-7-amine (0.520 g, 1.45 mmol), tert-butyl 4-oxo-3-(trifluoromethyl)piperidine-1-carboxylate (0.310 g, 1.16 mmol), Dibutyltin dichloride (0.070 g, 230.38 umol) and THF (4 mL). The reaction mixture was stirred at rt for 1 h. Then phenylsilane (0.631 g, 5.83 mmol) was added. The mixture was stirred at 60° C. for 16 h. The mixture was concentrated to afford the crude product. The crude product was purified by C18 column eluted with ACN/H₂O (v/v=4/1), filtered and concentrated under vacuum. This resulted in 0.247 g, (35.00% yield) of tert-butyl 4-((2-iodo-3-(2,2,2-trifluoroethyl)benzo[b]thiophen-7-yl)amino)-3-(trifluoromethyl)piperidine-1-carboxylate as yellow oil. LCMS: m/z=609 [M+1]⁺.

### Step 2. N-(2-iodo-3-(2,2,2-trifluoroethyl)benzo[b]thiophen-7-yl)-3-(trifluoromethyl)piperidin-4-amine

Into a 25-mL round-bottom flask purged and maintained with an inert atmosphere of nitrogen, was placed tert-butyl 4-((2-iodo-3-(2,2,2-trifluoroethyl)benzo[b]thiophen-7-yl)amino)-3-(trifluoromethyl)piperidine-1-carboxylate (0.243 g, 399.42 umol), ethyl acetate (2 mL) and HCl in EA solution (3 mL, 4 M). The reaction mixture was stirred at room temperature for 1.5 h. The residue was dissolved in saturated NaHCO₃ solution (20 mL). The resulting solution was extracted with EA (3×30 mL) and the organic layers were combined, washed with brine (2×20 mL), dried over anhydrous sodium sulfate, filtered and concentrated under reduce d pressure to afford desired product. This resulted in 0.220 g (108.36% yield) of N-(2-iodo-3-(2,2,2-trifluoroethyl)benzo[b]thiophen-7-yl)-3-(trifluoromethyl)piperidin-4-amine as yellow oil. LCMS: m/z=509 [M+1]⁺.

### Step 3. N-(2-iodo-3-(2,2,2-trifluoroethyl)benzo[b]thiophen-7-yl)-1-methyl-3-(trifluoromethyl)piperidin-4-amine

Into a 25-mL three necked bottle purged and maintained with an inert atmosphere of nitrogen, was placed N-(2-iodo-3-(2,2,2-trifluoroethyl)benzo[b]thiophen-7-yl)-3-(trifluoromethyl)piperidin-4-amine (0.212 g, 417.10 umol), Paraformaldehyde (0.081 g, 2.69 mmol), Acetic acid (0.2 mL), MeOH (2 mL) was added. The mixture was stirred at rt for 2 h. Sodium cyanoboronhydride, (0.189 g, 4.40 mmol) was added. The reaction mixture was stirred at room temperature for 1 h. The resulting solution was extracted with EA (3×30 mL) and the organic layers were combined, washed with brine (2×20 mL), dried over anhydrous sodium sulfate, filtered and concentrated under reduced pressure to afford desired product. This resulted in 0.173 g (79. 41% yield) of N-(2-iodo-3-(2,2,2-trifluoroethyl)benzo[b]thiophen-7-yl)-1-methyl-3-(trifluoromethyl)piperidin-4-amine as yellow oil. LCMS: m/z=523 [M+1]⁺.

### Step 4. (3-methoxy-4-((3-(7-((1-methyl-3-(trifluoromethyl)piperidin-4-yl)amino)-3-(2,2,2-trifluoroethyl)benzo[b]thiophen-2-yl)prop-2-yn-1-yl)amino)phenyl)dimethylphosphine oxide

Into a 10-mL round-bottom flask was placed N-(2-iodo-3-(2,2,2-trifluoroethyl)benzo[b]thiophen-7-yl)-1-methyl-3-(trifluoromethyl)piperidin-4-amine (0.162 g, 310.17 μmol), (3-methoxy-4-(prop-2-yn-1-ylamino)phenyl)dimethylphosphine oxide (0.330 g, 1.39 mmol), Pd(PPh₃)₂Cl₂ (0.055 g, 77.91 μmol), CuI (0.026 g, 136.51 μmol), DIEA (0.194 g, 1.50 mmol), methyl sulfoxide (3 mL). The reaction was stirred under nitrogen atmosphere at RT for 16 h. The reaction mixture was filtered through celite pad and celite pad was washed with A CN (3×20 mL). The filterate was then concentrated under reduced pressure to afford the crude product. The crude product was purified by preparative HPLC (Mobile Phase A: water (0.1% ammonium hydroxide), Mobile Phase B: ACN; Flow rate: 70 mL/min; Gradient: 40-70-90% B (2-30-60 min); 260 nm; RT: 32.948-36.438) to provide the desired product. This resulted in 0.120 g (62.51% yield) of (3-methoxy-4-((3-(7-((1-met hyl-3-(trifluoromethyl)piperidin-4-yl)amino)-3-(2,2,2-trifluoroethyl)benzo[b]thiophen-2-yl)prop-2-yn-1-yl)amino)phenyl)dimethylphosphine oxide as white solid. LCMS: m/z=632 [M+1]⁺.

### Step 5. (3-methoxy-4-((3-(7-(((Z)-1-methyl-3-(trifluoromethyl)piperidin-4-yl)amino)-3-(2,2,2-trifluoroethyl)benzo[b]thiophen-2-yl)prop-2-yn-1-yl)amino)phenyl)dimethylphosphine oxide (racemic) (169) and (3-methoxy-4-((3-(7-(((E)-1-methyl-3-(trifluoromethyl)piperidin-4-yl)amino)-3-(2,2,2-trifluoroethyl)benzo[b]thiophen-2-yl)prop-2-yn-1-yl)amino)phenyl)dimethylphosphine oxide (racemic) (170)

The sample (3-methoxy-4-((3-(7-((1-methyl-3-(trifluoromethyl)piperidin-4-yl)amino)-3-(2,2,2-trifluoroethyl)benzo[b]thiophen-2-yl)prop-2-yn-1-yl)amino)phenyl)dimethylphosphine oxide (0.120 g, 189.87 μmol) was separated by prep-chiral (Column:SA, Mobile Phase A: n-Hexane, Mobile Phase B: EtOH; Flow rate: 20 mL/min; EtOH=50:50:220 nm; RT: 6.987 min) to provide 0.044 g (22.45% yield) (3-methoxy-4-((3-(7-(((Z)-1-methyl-3-(trifluoromethyl)piperidin-4-yl)amino)-3-(2,2,2-trifluoroethyl)benzo[b]thiophen-2-yl)prop-2-yn-1-ylamino)phenyl)dimethylphosphine oxide (racemic) (169) as white solid. LCMS: m/z=632 [M+1]⁺

¹H NMR (400 MHz, DMSO-d₆) δ 7.35-7.11 (m, 4H), 6.91-6.76 (m, 2H), 6.08 (t, J=6.4 Hz, 1H), 5.16-5.04 (m, 1H), 4.34 (d, J=6.3 Hz, 2H), 4.08 (s, 1H), 3.82 (d, J=24.5 Hz, 5H), 2.87 (d, J=53.5 Hz, 2H), 2.50-2.36 (m, 2H), 2.43-2.33 (m, 1H), 2.26 (s, 3H), 1.89 (d, J=14.8 Hz, 1H), 1.75-1.72 (m, 1H), 1.58 (d, J=13.1 Hz, 6H). The sample (3-methoxy-4-((3-(7-((1-methyl-3-(trifluoromethyl)piperidin-4-yl)amino)-3-(2,2,2-trifluoroethyl)benzo[b]thiophen-2-yl)prop-2-yn-1-yl)amino)phenyl)dimethylphosphine oxide (0.120 g, 189.87 μmol) was separated by prep-chiral (Column:SA, Mobile Phase A: n-Hexane, Mobile Phase B: EtOH; Flow rate: 20 mL/min; EtOH=50:50; 220 mi; RT: 8.065 mm) to provide 0.040 g (20.41% yield) (3-methoxy-4-((3-(7-(((E)-1-methyl-3-(trifluoromethyl)piperidin-4-yl)amino)-3-(2,2,2-trifluoroethyl)benzo[b]thiophen-2-yl)prop-2-yn-1-yl)amino)phenyl)di methylphosphine oxide (racemic) (170) as white solid. LCMS: m/z=632 [M+1]⁺.

¹H NMR (400 MHz, DMSO-d₆) δ 7.41-7.12 (m, 4H), 6.90-6.78 (m, 2H), 6.09 (t, J=6.3 Hz, 1H), 5.09 (d, J=8.5 Hz, 1H), 4.34 (d, J=6.3 Hz, 2H), 4.07 (s, 1H), 3.82 (d, J=24.8 Hz, 5H), 2.97-2.70 (m, 2H), 2.55-2.51 (s, 2H), 2.33 (s, 1H), 2.24 (s, 3H), 1.89 (d, J=14.8 Hz, 1H), 1.75-7.72 (m, 1H), 1.58 (d, J=13.1 Hz, 6H).

### Example 171

### (2-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-5-(methylsulfonyl)phenyl)dimethylphosphine oxide (171)

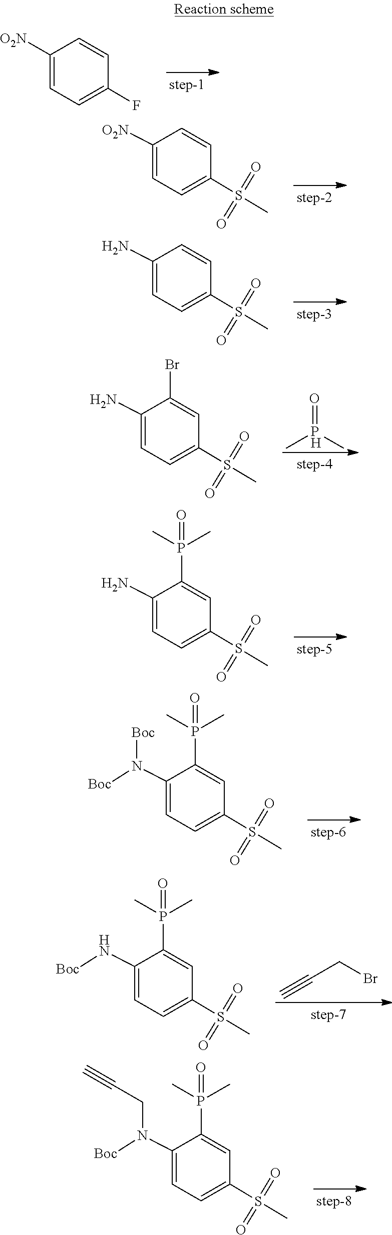

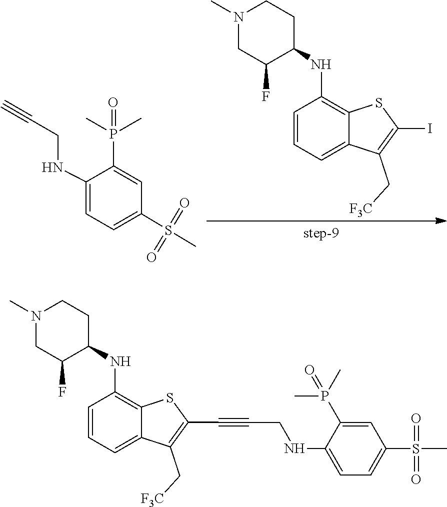

### Experiment Details

### Step 1. 1-(methylsulfonyl)-4-nitrobenzene

Into a 50 mL round-bottom flask was placed 1-fluoro-4-nitrobenzene (2.01 g, 14.26 mmol), Sodium methanesulfinate (1.80 g, 17.61 mmol), DMA (15 mL). The reaction was stirred at 100° C. for 4 h. The reaction was added to water slowly at R.T. The mixture was stirred at R.T for 1 h. The mixture was filtered and the filter cake was oven-dry. This resulted in 2.565 g (89.40% yield) of 1-(methylsulfonyl)-4-nitrobenzene as light-yellow solid. LCMS: m/z=202 [M+1]⁺.

### Step 2. 4-(methylsulfonyl)aniline

Into a 50 mL round-bottom flask was placed 1-(methylsulfonyl)-4-nitrobenzene (2.49 g, 12.39 mmol), NH₄Cl (6.73 g, 125.89 mmol), Iron (3.58 g, 64.12 mmol), EtOH (20 mL), water (10 mL). The mixture was stirred at 80° C. for 3 h. The mixture was add brine (200 mL), extracted with DCM/MeOH=5:1 (200 mL×2). The organic layers were dried over anhydrous sodium sulfate, filtered and concentrated under vacuum. This resulted in 1.960 g (crude) of 4-(methylsulfonyl)aniline as yellow solid. LCMS: m/z=172 [M+1]⁺.

### Step 3. 2-bromo-4-(methylsulfonyl)aniline

Into a 50 mL round-bottom flask was placed 4-(methylsulfonyl)aniline (1.00 g, 5.81 mmol), NBS (0.99 g, 5.56 mmol), DMF (10 mL). The reaction was stirred at 20° C. overnight. The residue purified by flash-C18 eluted with ACN/water (v/v=3/7) to provide the desired product. This resulted in 1.055 g (72.58% yield) of 2-bromo-4-(methylsulfonyl)aniline as light-yellow solid. LCMS: m/z=250 [M+1]⁺.

### Step 4. (2-amino-5-(methylsulfonyl)phenyl)dimethylphosphine oxide

Into a 50 mL three round bottom flask was placed 2-bromo-4-(methylsulfonyl)aniline as (0.52 g, 2.08 mmol), Palladium (II) acetate (0.09 g, 0.38 mmol), Dimethylbisdiphenylphosphinoxanthene (0.25 g, 0.43 mmol), DIEA (1.31 g, 10.16 mmol), DMF (10 mL). The reaction was heat to 130° C. under nitrogen. Dimethylphosphine oxide (0.50 g, 6.44 mmol) was added to the reaction slowly. The mixture was stirred at 130° C. for 1 h. The reaction was quenched with water (50 mL), extracted with EA (2×50 mL). The organic layers were combined, washed with brine (2×100 mL), dried over anhydrous sodium sulfate, filtered and concentrated under vacuum. The residue purified by flash-C18 eluted with ACN/water (v/v=1/9) to provide the desired product. This resulted in 0.343 g (66.60% yield) of (2-amino-5-(methylsulfonyl)phenyl)dimethylphosphine oxide as light-yellow solid. LCMS: m/z=248 [M+1]⁺.

### Step 5. tert-butyl N-tert-butoxycarbonyl-N-(2-dimethylphosphoryl-4-methylsulfonyl-phenyl)carbamate

Into a 8 mL vial was placed (2-amino-5-(methylsulfonyl)phenyl)dimethylphosphine oxide (0.30 g, 1.20 mmol), Di-tert-butyl dicarbonate (0.84 g, 3.85 mmol), N-(4-pyridyl)dimethylamine (0.54 g, 4.38 mmol), 1,4-Dioxane (6 mL). The reaction was stirred at 110° C. for 1 h. The mixture was concentrated under vacuum. This resulted in 0.56 g (crude) of tert-butyl N-tert-butoxycarbonyl-N-(2-dimethylphosphoryl-4-methylsulfonyl-phen yl)carbamate as semi-solid. LCMS: m/z=448 [M+1]⁺.

### Step 6. tert-butyl (2-(dimethylphosphoryl)-4-(methylsulfonyl)phenyl)carbamate

Into a 50 mL round-bottom flask was placed tert-butyl N-tert-butoxycarbonyl-N-(2-dimethylphosphoryl-4-methylsulfonyl-phenyl)carbamate (0.54 g, 1.21 mmol), K₂CO₃ (0.45 g, 3.26 mmol), MeOH (10 mL). The reaction was stirred at 70° C. for 1 h. The mixture was concentrated under vacuum. The reaction was quenched by water (50 mL), extracted with EA (2×30 mL). The organic layers were combined, washed with brine (2×50 mL), dried over anhydrous sodium sulfate, filtered and concentrated under vacuum. The residue purified by flash-C18 eluted with ACN/water (v/v=3/7) to provide the desired product. This resulted in 0.271 g (64.65% yield) of tert-butyl (2-(dimethylphosphoryl)-4-(methylsulfonyl)phenyl)carbamate as off-white solid. LCMS: m/z=348 [M+1]⁺.

### Step 7. tert-butyl (2-(dimethylphosphoryl)-4-(methylsulfonyl)phenyl)(prop-2-yn-1-yl)carbamate

Into a 50 mL round-bottom flask was placed tert-butyl (2-(dimethylphosphoryl)-4-(methylsulfonyl)phenyl)carbamate (0.27 g, 0.78 mmol), THF (10 mL). The reaction was stirred at 0° C. NaH (0.09 g, 3.92 mmol) was added to the reaction slowly at 0° C. and stirred for 0.5 h. 3-bromoprop-1-yne (1.00 g, 8.44 mmol) was added to the reaction and stirred at 60° C. overnight. The reaction was quenched by water (50 mL), extracted with EA (2×30 mL). The organic layers were concentrated under vacuum. This resulted in 0.959 g (crude) of tert-butyl(2-(dimethylphosphoryl)-4-(methylsulfonyl)phenyl)(prop-2-yn-1-yl)carbamate as brown oil. LCMS: m/z=386 [M+1]⁺.

### Step 8. dimethyl(5-(methylsulfonyl)-2-(prop-2-yn-1-ylamino)phenyl)phosphine oxide

Into a 50 mL round-bottom flask was placed tert-butyl(2-(dimethylphosphoryl)-4-(methylsulfonyl)phenyl)(prop-2-yn-1-yl)carbamate (0.96 g, 2.49 mmol), TFA (2 mL), DCM (10 mL). The reaction was stirred at R.T for 0.5 h. The reaction was concentrated under vacuum. The mixture purified by flash-C18 eluted with ACN/water (v/v=1/4) to provide the desired product. This resulted in 0.098 g (13.81% yield) of dimethyl(5-(methylsulfonyl)-2-(prop-2-yn-1-ylamino)phenyl)phosphine oxide as yellow solid. LCMS: m/z=286 [M+1]⁺.

### Step 9. (2-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-5-(methylsulfonyl)phenyl)dimethylphosphine oxide (171)

Into a 8 mL vial was placed (3S,4R)-3-fluoro-N-(2-iodo-3-(2,2,2-trifluoroethyl)benzo[b]thiophen-7-yl)-1-methylpiperidin-4-amine (0.13 g, 0.28 mmol), dimethyl(5-(methylsulfonyl)-2-(prop-2-yn-1-ylamino)phenyl)phosphine oxide (0.10 g, 0.33 mol), bis(triphenylphosphine)palladium(II) chloride (0.04 g, 58.08 μmol), CuI (0.02 g, 0.12 mmol), DIEA (0.12 g, 0.90 mmol), Methyl sulfoxide (6 mL). The reaction was stirred at 60° C. for 1 h under nitrogen. The reaction was quenched with water (30 mL), extracted with EA (2×20 mL). The organic layers were combined, washed with brine (2×50 mL), dried over anhydrous sodium sulfate, filtered and concentrated under vacuum. The mixture was purified by preparative HPLC (Mobile Phase A: water (10 mmol/L NH₄OH), Mobile Phase B: ACN; Flow rate: 70 mL/min; Gradient: 35-69-69% B (2-30-35 min); 266 nm; RT: 30.65-32.80 min) to provide the desired product. This resulted in 0.121 g (68.75% yield) of (2-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-5-(methylsulfonyl)phenyl)dimethylphosphine oxide (171) as off-white solid. LCMS: m/z=630 [M+1]⁺.

¹H NMR (400 MHz, DMSO-d₆) δ 8.55 (t, J=6.0 Hz, 1H), 7.85-7.76 (m, 2H), 7.32-7.19 (m, 2H), 7.06-7.00 (m, 1H), 6.79 (d, J=7.6 Hz, 1H), 5.19 (d, J=8.6 Hz, 1H), 4.80 (d, J=49.5 Hz, 1H), 4.51 (d, J=5.9 Hz, 2H), 3.92-3.79 (m, 2H), 3.65 (d, J=28.9 Hz, 1H), 3.14 (s, 3H), 3.03 (t, J=11.4 Hz, 1H), 2.79 (d, J=11.0 Hz, 1H), 2.18 (m, 4H), 2.08 (t, J=11.4 Hz, 1H), 2.00-1.89 (m, 1H), 1.79 (d, J=13.5 Hz, 6H), 1.72 (d, J=12.6 Hz, 1H).

### Example 172

(3S,4R)-3-fluoro-N-(2-(3-((2-methoxy-4-(1-methyl-1H-1,2,4-triazol-3-yl)phenyl)amino)prop-1-yn-1-yl)-3-(2,2,2-trifluoroethyl)benzo[b]thiophen-7-yl)-1-methylpiperidin-4-amine (172)

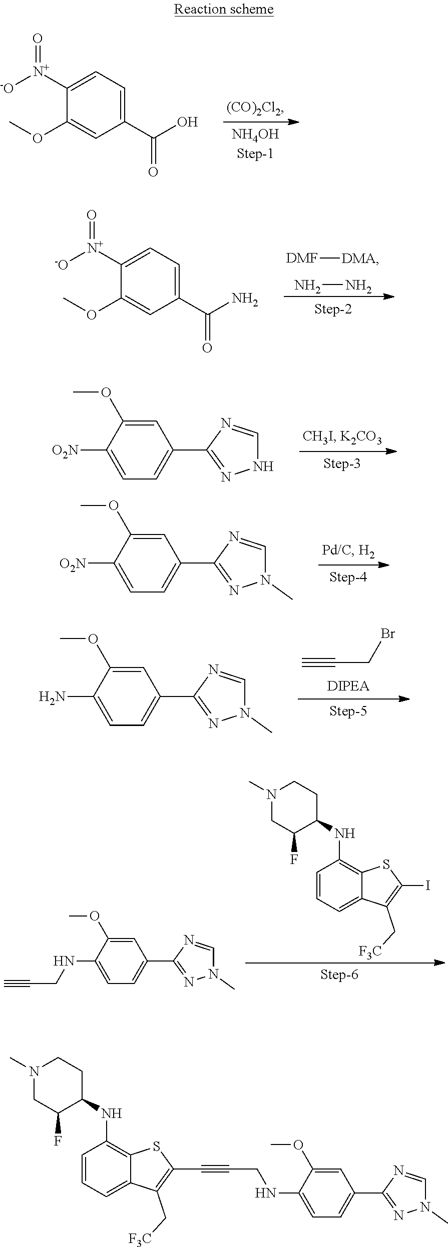

### Experiment Details

### Step 1. 3-methoxy-4-nitrobenzamide

Into an ice-cold 250 mL flask was placed 3-Methoxy-4-nitrobenzoic acid (10.03 g, 50.88 mmol), Oxalyl chloride (7.70 g, 60.67 mmol), DCM (50 mL), DMF (1 mL), and stirred for 2 h at Ox. The reaction quenched with ammonium hydroxide (10 mL) and concentrated. The residue was slurred with water (50 mL) for 0.5 h. The slurry solution was filtered, the filter cake was washed with water (10 mL) and dried under vacuum to give 3-methoxy-4-nitrobenzamide (13.03 g, 66.43 mmol, crude) as off-white solid. LCMS: m/z=197[M+1]⁺.

### Step 2. 3-(3-methoxy-4-nitrophenyl)-1H-1,2,4-triazole

Into a 50 mL flask was placed 3-methoxy-4-nitrobenzamide (3.18 g, 16.21 mmol), DMF-DMA (15 mL) and stirred for 1 h at 120° C. The reaction was cooled to room temperature and concentrated. The residue w as dissolved with HOAc (10 mL), hydrazinium hydroxide solution (0.5 mL) was added. The reaction was stirred for 4 h at 90° C. and concentrated. The residue was dissolved with water (50 mL), neutralized with 2M HCl (aq.), extracted with EA (20 mL×2). The combined organic layers were concentrated to give 3-(3-methoxy-4-nitro-phenyl)-1H-1,2,4-triazole (2.520 g, 11.44 mmol, crude) as light yellow solid. LCMS: m/z=221[M+1]⁺.

### Step 3. 3-(3-methoxy-4-nitrophenyl)-1-methyl-1H-1,2,4-triazole

Into a 50 mL flask was placed 3-(3-methoxy-4-nitro-phenyl)-1H-1,2,4-triazole (2.468 g, 11.21 mmol), Methyl Iodide (1.933 g, 13.62 mmol), K₂CO₃ (2.323 g, 16.81 mmol), DMF (10 mL). The reaction was stirred for 1 h at room temperature. The reaction was quenched with MeOH (2 mL) and concentrated. The residue was purified with C₁₈ column, eluted with ACN/water (v/v=1/3) to afford 3-(3-methoxy-4-nitro-phenyl)-1-methyl-1,2,4-triazole (0.515 g, 2.20 mmol, 19.62% yield) as light yellow solid. LCMS: m/z=235[M+1]⁺

### Step 4. 2-methoxy-4-(1-methyl-1H-1,2,4-triazol-3-yl)aniline

Into a 50 mL flask was placed 3-(3-methoxy-4-nitro-phenyl)-1-methyl-1,2,4-triazole (0.499 g, 2.13 mmol), Pd/C (0.221 g, 2.08 mmol), MeOH (5 mL). The reaction was stirred overnight at Hydrogen atmosphere, room temperature. The reaction was filtered, the filter cake was washed with MeOH (5 mL×2). The filtrate was concentrated to afford 2-methoxy-4-(1-methyl-1,2,4-triazol-3-yl)aniline (0.511 g, 2.50 mmol, crude) as off-white solid. LCMS: m/z=205[M+1]⁺

### Step 5. 2-methoxy-4-(1-methyl-1H-1,2,4-triazol-3-yl)-N-(prop-2-yn-1-yl)aniline

Into a 50 mL flask was placed 2-methoxy-4-(1-methyl-1,2,4-triazol-3-yl)aniline (0.446 g, 2.19 mmol), 3-bromoprop-1-yne (0.288 g, 2.42 mmol), DIEA (0.499 g, 3.86 mmol), DMF (5 mL). The reaction was stirred overnight at 50° C. The reaction was purified with C₁₈ column, eluted with ACN/water (v/v=1/3) to afford 2-methoxy-4-(1-methyl-1,2,4-triazol-3-yl)-N-prop-2-ynyl-aniline (0.335 g, 1.38 mmol, 63.32% yield) as light brown solid. LCMS: m/z=243[M+1]⁺

### Step 6. (3S,4R)-3-fluoro-N-(2-(3-((2-methoxy-4-(1-methyl-1H-1,2,4-triazol-3-yl)phenyl)amino)prop-1-yn-1-yl)-3-(2,2,2-trifluoroethyl)benzo[b]thiophen-7-yl)-1-methylpiperidin-4-amine (172)

Into a 4 mL vial purged and maintained with nitrogen atmosphere was placed (3S,4R)-3-fluoro-1-methyl-N-(3-(2,2,2-trifluoroethyl)benzo[b]thiophen-7-yl)piperidin-4-amine (0.099 g, 209.62 μmol), 2-methoxy-4-(1-methyl-1H-1,2,4-triazol-3-yl)-N-(prop-2-yn-1-yl)aniline (0.078 g, 321.95 μmol), Pd(PPh₃)₂Cl₂ (0.015 g, 21.25 μmol), CuI (0.015 g, 78.76 μmol), TEA (0.033 g, 326.1211 μmol), DMF (1 mL), and stirred for 2 h at room temperature. The reaction was quenched with water (4 mL), extracted with EA (3 mL×2). The combined organic layers were washed with water (4 mL) and brine (4 mL) successively, separated, then concentrated with vacuum. The residue was purified with prep-HPLC (Mobile Phase A: water (ammonium hydroxide), Mobile Phase B: ACN; Flow rate: 70 mL/min; Gradient: 35-70-95% B (2-30-60 min); 270 nm; RT: 35.218-37.222 min) to afford (3S,4R)-3-fluoro-N-(2-(3-((2-methoxy-4-(1-methyl-1H-1,2,4-triazol-3-yl)phenyl)amino)prop-1-yn-1-yl)-3-(2,2,2-trifluoroethyl)benzo[b]thiophen-7-yl)-1-methylpiperidin-4-amine (172) (0.077 g, 131.25 μmol, 62.62% yield) as a white solid. LCMS: m/z=587 [M+1]⁺.

¹H NMR (400 MHz, DMSO-d₆) δ 8.40 (s, 1H), 7.55-7.50 (m, 1H), 7.43 (d, J=1.7 Hz, 1H), 7.30-7. 23 (m, 1H), 7.20 (d, J=8.0 Hz, 1H), 6.86-6.73 (m, 2H), 5.82 (t, J=6.4 Hz, 1H), 5.16 (d, J=8.5 Hz, 1H), 4.79 (d, J=49.5 Hz, 1H), 4.38-4.29 (m, 2H), 3.87 (d, J=8.2 Hz, 5H), 3.83-3.76 (m, 2H), 3.72-3.55 (m, 2H), 3.02 (t, J=11.4 Hz, 1H), 2.79 (d, J=11.1 Hz, 1H), 2.18 (s, 4H), 2.12-2.03 (m, 1H), 2.00-1.88 (m, 1H), 1.71 (d, J=12.3 Hz, 1H).

### Example 173

### (3S,4R)-3-fluoro-N-(2-(3-((4-(5-fluoropyrimidin-2-yl)-2-methoxyphenyl)amino)prop-1-yn-1-yl)-3-(2,2,2-trifluoroethyl)benzo[b]thiophen-7-yl)-1-methylpiperidin-4-amine (173)

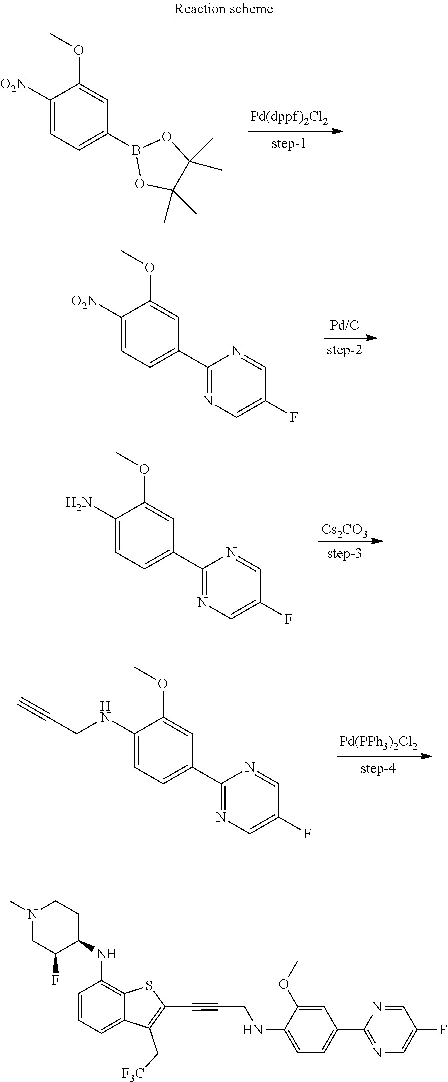

### Experimental Details

### Step 1. 5-fluoro-2-(3-methoxy-4-nitrophenyl)pyrimidine

Into a 100-mL three necked bottle purged and maintained with an inert atmosphere of nitrogen, was placed 2-(3-methoxy-4-nitrophenyl)-4,4,5,5-tetramethyl-1,3,2-dioxaborolane (1.188 g, 4.26 mmol), 2-bromo-5-fluoro pyrimidine (1.018 g, 5.75 mmol), Pd(dppf)₂Cl₂ (0.537 g, 0.73 mmol), Na₂CO₃ (1.226 g, 11.57 mmol), Diox (10 mL), H₂O (2 mL). The reaction mixture was stirred at 90° C. for 4 h. The resulting solution was added to water (30 mL). The resulting solution was extracted with EA (2×15 mL). The organic layers combined and washed with brine (20 mL), dried over anhydrous Na₂SO₄, the residue was concentrated under vacuum. The crude product purified by washing with HEX/EA (v/v=20/1). This resulted in 0.967 g (91.16% yield) of 5-fluoro-2-(3-methoxy-4-nitrophenyl)pyrimidine as yellow solid. LCMS: m/z=250 [M+1]⁺.

### Step 2. 4-(5-fluoropyrimidin-2-yl)-2-methoxyaniline

Into a 100-mL three necked bottle was placed 5-fluoro-2-(3-methoxy-4-nitrophenyl)pyrimidine (1.153 g, 4.63 mmol), Pd/C (0.849 g, 7.98 mmol), MeOH (20 mL). The reaction mixture was stirred at 25° C. for 4 h. The reaction solution was filtered, and the filtrate was collected, concentrated under vacuum. This resulted in 0.76 g (74.93% yield) of 4-(5-fluoropyrimidin-2-yl)-2-methoxyaniline as yellow solid. LCMS: m/z=220 [M+1]⁺.

### Step 3. 4-(5-fluoropyrimidin-2-yl)-2-methoxy-N-(prop-2-yn-1-yl)aniline

Into a 50-mL flask was placed 4-(5-fluoropyrimidin-2-yl)-2-methoxyaniline (0.299 g, 1.36 mmol), 3-Bromopropyne (0.243 g, 2.04 mmol), Cs₂CO₃ (0.517 g, 1.59 mmol), KI (0.225 g, 1.36 mmol), DMF (5 mL). The reaction mixture was stirred at 80° C. for 16 h. LCMS showed that the reaction was completed. The reaction mixture was purified by C18 column eluted with ACN/H₂O (v/v=2/8). This resulted in 0.057 g (16.24% yield) of 4-(5-fluoropyrimidin-2-yl)-2-methoxy-N-(prop-2-yn-1-yl)aniline as red solid. LCMS: m/z=258 [M+1]⁺.

### Step 4. (3S,4R)-3-fluoro-N-(2-(3-((4-(5-fluoropyrimidin-2-yl)-2-methoxyphenyl)amino)prop-1-yn-1-yl)-3-(2,2,2-trifluoroethyl)benzo[b]thiophen-7-yl)-1-methylpiperidin-4-amine (173)

Into a 50-mL three necked bottle and maintained with an inert atmosphere of nitrogen, was placed (3S,4R)-3-fluoro-N-(2-iodo-3-(2,2,2-trifluoroethyl)benzo[b]thiophen-7-yl)-1-methylpiperidin-4-amine (0.062 g, 0.13 m mol), 4-(5-fluoropyrimidin-2-yl)-2-methoxy-N-(prop-2-yn-1-yl)aniline (0.066 g, 0.26 mmol), CuI (0.012 g, 0.063 mmol), Pd(PPh₃)₂Cl₂ (0.020 g, 0.028 mmol), DIEA (0.104 g, 0.81 mmol) and DMSO (5 mL). The reaction mixture was stirred at room temperature for 1 h. LCMS showed that the reaction was completed. The mixture was purified by preparative HPLC (Mobile Phase A: water (10 mmoL/L ammonium hydroxide), Mobile Phase B: ACN; Flow rate: 40 mL/min; Gradient: 25-75-75% B (2-30-60 min); 266 nm; RT: 52.053-53.667 min). This resulted in 0.027 g (34.19% yield) of (3S,4R)-3-fluoro-N-(2-(3-((4-(5-fluoropyrimidin-2-yl)-2-methoxyphenyl)amino)prop-1-yn-1-yl)-3-(2,2,2-trifluoroethyl)benzo[b]thiophen-7-yl)-1-methylpiperidin-4-amine (173) as off white solid. LCMS: m/z=602 [M+1]⁺.

¹H NMR (400 MHz, DMSO) δ 8.85 (d, J=4.9 Hz, 2H), 7.93 (d, J=8.5 Hz, 1H), 7.81 (s, 1H), 7.26 (t, J=7.8 Hz, 1H), 7.19 (d, J=7.8 Hz, 1H), 6.87 (d, J=8.4 Hz, 1H), 6.77 (d, J=7.6 Hz, 1H), 6.13 (t, J=6.3 Hz, 1H), 5.16 (d, J=8.6 Hz, 1H), 4.79 (d, J=49.3 Hz, 1H), 4.38 (d, J=6.2 Hz, 2H), 4.10 (d, J=6.6 Hz, 1H), 3.89 (s, 3H), 3.82 (d, J=11.0 Hz, 1H), 3.73-3.52 (m, 1H), 3.02 (t, J=10.5 Hz, 1H), 2.79 (d, J=11.6 Hz, 1H), 2.36-2.22 (m, 1H), 2.18 (s, 3H), 2.07 (t, J=11.2 Hz, 1H), 1.99-1.85 (m, 1H), 1.71 (d, J=11.8 Hz, 1H).

### Example 174

### (4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-(methoxy-d₃)phenyl)dimethylphosphine oxide (174)

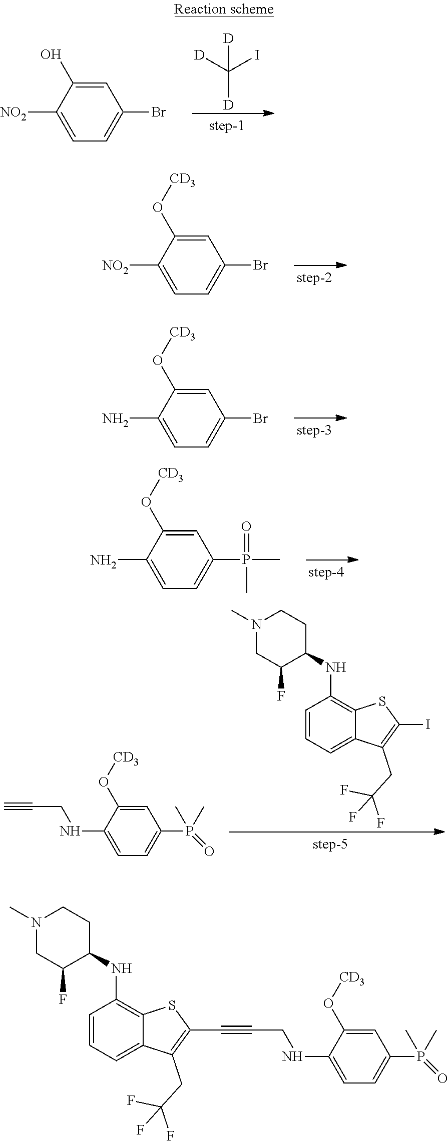

### Experiment Details

### Step 1. 4-bromo-2-(methoxy-d₃)-1-nitrobenzene

Into a 250 mL round-bottom flask was placed 5-bromo-2-nitrophenol (4.68 g, 21.47 mmol), iodomethane-d 3 (6.17 g, 42.56 mmol), DIEA (3.93 g, 30.41 mmol), DMF (50 mL). The reaction was stirred at 50° C. overnight. The reaction was quenched with water (100 mL), extracted with EA (3×100 mL). The organic layers were combined, washed with brine (2×500 mL), dried over anhydrous sodium sulfate, filtered and concentrated under vacuum. The mixture was purified by silica gel chromatography eluted with EA/hexane (V/V=1:4) to provide the desired product. This resulted in 4.62 g (92.56% yield) of 4-bromo-2-(methoxy-d3)-1-nitrobenzene as yellow solid. LCMS: m/z=235 [M+1]⁺.

### Step 2. 4-bromo-2-(methoxy-d₃)aniline

Into a 250 mL round-bottom flask was placed 4-bromo-2-(methoxy-d₃)-1-nitrobenzene (3.24 g, 13.77 mmol), Iron (4.21 g, 75.44 mmol), NH₄Cl (7.90 g, 147.70 mmol), EtOH (60 mL), Water (30 mL). The reaction was stirred at 85° h for 1 h. The reaction was quenched with water (200 mL), extracted with EA (3×200 mL). The organic layers were combined, washed with brine (2×200 mL), dried over anhydrous sodium sulfate, filtered and concentrated under vacuum. The mixture was purified by silica gel chromatography eluted with EA/hexane (v/v=3:7) to provide the desired product. This resulted in 1.52 g (53.82% yield) of 4-bromo-2-(methoxy-d₃)aniline as brown solid. LCMS: m/z=205 [M+1]⁺.

### Step 3. (4-amino-3-(methoxy-d₃)phenyl)dimethylphosphine oxide

Into a 40 mL vial was placed 4-bromo-2-(methoxy-d₃)aniline (1.49 g, 7.29 mmol), Palladium (II) acetate (0.42 g, 1.87 mmol), Dimethylbisdiphenylphosphinoxanthene (0.92 g, 1.59 mmol), N,N-Diisopropylethylamine (2.20 g, 17.00 mmol), DMF (15 mL). The reaction was stirred at 120° C. for 0.5 h under nitrogen. Dimethylphosphine oxide (1.86 g, 23.86 mmol) was added to the mixture slowly. The reaction was stirred at 120° C. for 1 h under nitrogen. The residue purified by flash-C18 eluted with ACN/water (v/v=1/9) to provide the desired product. This resulted in 0.967 g (65.64% yield) of (4-amino-3-(methoxy-d3)phenyl)dimethylphosphine oxide as yellow semi-solid. LCMS: m/z=203 [M+1]⁺.

### Step 4. (3-(methoxy-d₃)-4-(prop-2-yn-1-ylamino)phenyl)dimethylphosphine oxide

Into a 100 mL round-bottom flask was placed methyl (4-amino-3-(methoxy-d₃)phenyl)dimethylphosphine ox ide (0.50 g, 2.46 mmol), 3-bromoprop-1-yne (0.36 g, 3.05 mmol), DIEA (0.60 g, 4.60 mmol) and NMP (30 mL). The reaction was stirred at 50° C. for 6 h. The residue purified by flash-C18 eluted with ACN/water (v/v=3/7) to provide the desired product. This resulted in 0.274 g (46.4% yield) of (3-(methoxy-d₃)-4-(prop-2-yn-1-ylamino)phenyl)dimethylphosphine oxide as yellow oil. LCMS: m/z=241[M+1]⁺.

### Step 5. (4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-(methoxy-d₃)phenyl)dimethylphosphine oxide (174)

Into a 8 mL vial was placed (3S,4R)-3-fluoro-N-(2-iodo-3-(2,2,2-trifluoroethyl)benzo[b]thiophen-7-yl)-1-methylpiperidin-4-amine (0.12 g, 0.25 mmol), (3-(methoxy-d₃)-4-(prop-2-yn-1-ylamino)phenyl)dimethylphosphine oxide (0.08 g, 0.34 mmol), bis(triphenylphosphine)palladium(II) chloride (0.03 g, 45.33 μmol), CuI (0.01 g, 57.76 μmol), DIEA (0.14 g, 1.08 mmol), Methyl sulfoxide (2 mL). The reaction was stirred at 60° C. for 1 h under nitrogen. The reaction was quenched with water (20 mL), extracted with EA (3×30 mL). The organic layers were combined, washed with brine (50 mL), dried over anhydrous sodium sulfate, filtered and concentrated under vacuum. The mixture was purified by preparative HPLC (Mobile Phase A: water (10 mmol/L ammonium hydroxide), Mobile Phase B: ACN; Flow rate: 70 mL/min; Gradient: 20-45-45% B (2-30-34 min); 266 in; RT: 28.10-33.71 min) to provide the desired product. This resulted in 0.0447 g (30.86% yield) of (4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-(methoxy-d₃)phenyl)dimethylphosphine oxide (174) as light-yellow solid. LCMS: m/z=585 [M+1]⁺.

¹H NMR (400 MHz, Methanol-d₄) δ 7.31-7.23 (m, 2H), 7.21-7.15 (m, 2H), 6.96-6.90 (m, 1H), 6.76 (d, J=7.7 Hz, 1H), 4.88 (s, 1H), 4.36 (s, 2H), 3.77-3.59 (m, 3H), 3.18 (t, J=11.7 Hz, 1H), 2.91 (d, J=11.8 Hz, 1H), 2.38 (d, J=13.3 Hz, 1H), 2.29 (s, 3H), 2.27-2.17 (m, 1H), 1.98-1.89 (m, 2H), 1.73 (d, J=13.3 Hz, 6H).

### Example 175

### (4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-(methylsulfonyl)phenyl)dimethylphosphine oxide (175)

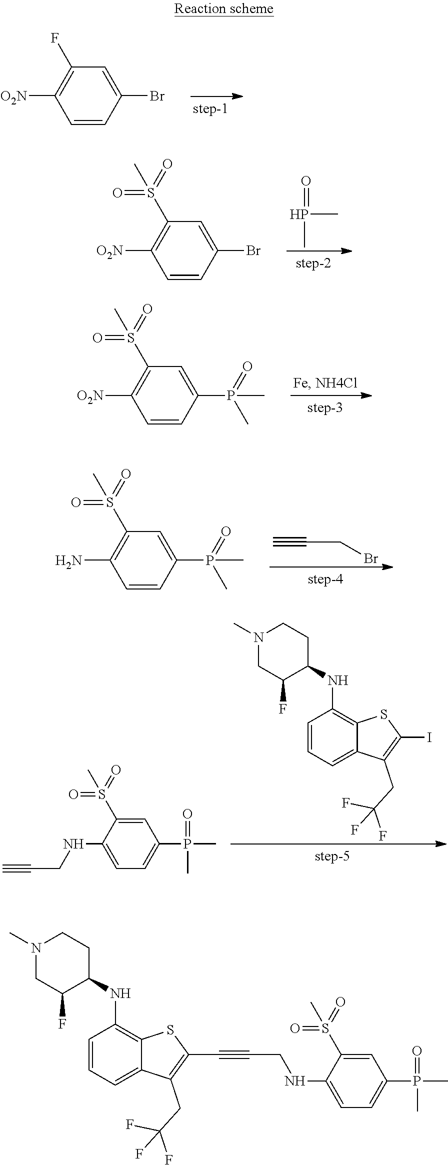

### Experimental Details

### Step 1. 4-bromo-2-(methylsulfonyl)-1-nitrobenzene

Into a 100-mL three necked bottle purged and maintained with an inert atmosphere of nitrogen, was placed 4-bromo-2-fluoro-1-nitrobenzene (2.18 g, 9.91 mmol), Sodium methanesulfinate (3.17 g, 31.05 mmol), DMSO (200 mL). The reaction mixture was stirred at 80° C. for 3 h. The resulting solution was added to water (100 mL). The resulting solution was extracted with EA (2×150 mL). The organic layers combined and washed with brine (50 mL), dried over anhydrous Na₂SO₄, the residue was concentrated under vacuum. The residue was applied onto a silica gel column eluted with EA:hexane (v:v=1:1). This resulted in 2.50 g (90.07% yield) of 4-bromo-2-(methylsulfonyl)-1-nitrobenzene as brown solid. LCMS: m/z=280 [M+1]⁺

### Step 2. dimethyl(3-(methylsulfonyl)-4-nitrophenyl)phosphine oxide

Into a 40-mL reaction vial purged and maintained with an inert atmosphere of nitrogen, was placed 4-bromo-2-(methylsulfonyl)-1-nitrobenzene (1.27 g, 4.55 mmol), Pd(OAc)₂ (141.00 mg, 0.63 mmol), Xantphos (534.00 mg, 0.93 mmol), DIEA (1.57 g, 12.17 mmol), DMF (15 mL) and stirred at 110° C. for 5 min, then added Phosphine oxide (651.00 mg, 7.18 mmol) in DMF (5 mL). The reaction mixture was stirred at 110° C. for 2 h. The resulting solution was concentrated under vacuum. The residue was applied onto a C₁₈ column eluted with ACN/H₂O (0.01% TFA) (v:v=1:1), Flow rate: 100 mL/min. This resulted in 1.14 g (90.26% yield) of dimethyl(3-(methylsulfonyl)-4-nitrophenyl)phosphine oxide as yellow solid. LCMS: m/z=278 [M+1]⁺

### Step 3. (4-amino-3-(methylsulfonyl)phenyl)dimethylphosphine oxide

Into a 100-mL three necked bottle purged and maintained with an inert atmosphere of nitrogen, was placed dimethyl(3-(methylsulfonyl)-4-nitrophenyl)phosphine oxide (1.01 g, 3.64 mmol), Fe (850.00 mg, 15.22 m mol), NH₄Cl (830.00 mg, 15.51 mmol), ETOH (20 mL), H₂O (5 mL). The reaction mixture was stirred at 80° C. for 2 h. The reaction mixture was filtered and the filter cake was washed with EtOH (20 mL). The residue was concentrated under vacuum. The residue was applied onto a C₁₈ column eluted with ACN/H₂O (0.01% TFA) (v:v=1:1), Flow rate: 100 m/min. This resulted in 800.00 mg (88.81% yield) of (4-amino-3-(methylsulfonyl)phenyl)dimethylphosphine oxide as yellow solid. LCMS: m/z=248 [M+1]⁺

### Step 4. dimethyl(3-(methylsulfonyl)-4-(prop-2-yn-1-ylamino)phenyl)phosphine oxide

Into a 50-mL three necked bottle purged and maintained with an inert atmosphere of nitrogen, was placed (4-amino-3-(methylsulfonyl)phenyl)dimethylphosphine oxide (1.03 g, 4.16 mmol), 3-bromoprop-1-yne (740.00 mg, 6.22 mmol), Cs₂CO₃ (4.54 g, 13.94 mmol), NaI (337.00 mg, 2.28 mmol), ACN (50 mL). The reaction mixture was filtered and the filter cake was washed with ACN (20 mL). The residue was concentrated under vacuum. The residue was applied onto a C₁₈ column eluted with ACN/H₂O (0.01% TFA) (v:v=1:1), Flow rate: 100 mL/min. This resulted in 275.00 mg (23.16% yield) of dimethyl(3-(methylsulfonyl)-4-(prop-2-yn-1-ylamino)phenyl)phosphine oxide as brown solid. LCMS: m/z=286 [M+1]⁺

### Step 5. (4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-(methylsulfonyl)phenyl)dimethylphosphine oxide (175)

Into a 50-mL three necked bottle purged and maintained with an inert atmosphere of nitrogen, was placed (3S,4R)-3-fluoro-N-(2-iodo-3-(2,2,2-trifluoroethyl)benzo[b]thiophen-7-yl)-1-methylpiperidin-4-amine (83.00 mg, 0.17 mmol), dimethyl(3-(methylsulfonyl)-4-(prop-2-yn-1-ylamino)phenyl)phosphine oxide (225.00 mg, 0.78 mmol), Pd(PPh₃)₂Cl₂ (36.00 mg, 0.051 mmol), CuI (31.00 mg, 0.16 mmol), DIEA (227.00 mg, 1.75 mmol), DMSO (8 mL). The reaction mixture was stirred at 50° C. for 3 h. The residue was applied onto a C₁₈ column eluted with ACN/H₂O (0.01% TFA) (v:v=1:1), Flow rate: 80 mL/min. the residue was concentrated under vacuum. The resulting crude product was further purified by pre-HPLC with ACN/H₂O (0.1% ammonium hydroxide), Flow rate: 70 mL/min; Gradient: 30-65-65% B (2-30-34 min); 262 nm; RT: 29.68-31.312). This resulted in 36.00 mg (32.53% yield) of (4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-(methylsulfonyl)phenyl)dimethylphosphine oxide (175) as white solid. LCMS: m/z=630 [M+1]⁺

¹H NMR (400 MHz, DMSO-d₆) δ 8.08-8.00 (m, 1H), 7.90-7.85 (m, 1H), 7.30-7.25 (m, 1H), 7.25-7.18 (m, 2H), 7.03-6.95 (m, 1H), 6.79 (d, J=7.6 Hz, 1H), 5.20 (d, J=8.6 Hz, 1H), 4.80 (d, J=49.6 Hz, 1H), 4.57 (d, J=5.9 Hz, 2H), 3.90-3.83 (m, 2H), 3.65 (d, J=28.5 Hz, 1H), 3.20 (s, 3H), 3.07-3.00 (m, 1H), 2.80 (d, J=11.1 Hz, 1H), 2.30-2.14 (m, 4H), 2.10-2.02 (m, 1H), 2.01-1.87 (m, 1H), 1.75-1.66 (m, 1H), 1.62 (d, J=13.4 Hz, 6H).

### Example 179

### (5-((3-(7-(((3S,4R)-3-fluoro-1-(tetrahydro-2H-pyran-4-yl)piperidin-4-yl)amino)-3-(2,2,2-trifluoroethyl)benzo[b]thiophen-2-yl)prop-2-yn-1-yl)amino)-6-methoxypyridin-2-yl)dimethylphosphine oxide (179)

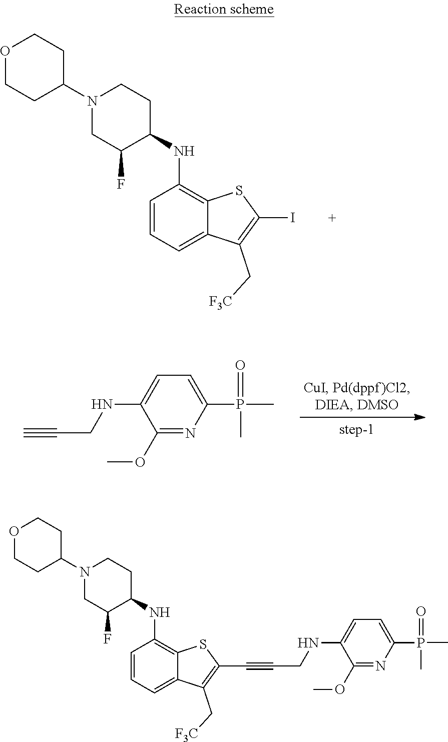 [SMILES: F[C@H]1CN(C2CCOCC2)CC[C@H]1Nc1cccc2c(CC(F)(F)F)c(I)sc12 ; C#CCNc1ccc(P(C)(C)=O)nc1OC ; COc1nc(P(C)(C)=O)ccc1NCC#Cc1sc2c(N[C@@H]3CCN(C4CCOCC4)C[C@@H]3F)cccc2c1CC(F)(F)F]

### Experiment Details

### Step 1. (5-((3-(7-(((3S,4R)-3-fluoro-1-(tetrahydro-2H-pyran-4-yl)piperidin-4-yl)amino)-3-(2,2,2-trifluoroethyl) benzo[b]thiophen-2-yl)prop-2-yn-1-yl)amino)-6-methoxypyridin-2-yl)dimethylphosphine oxide (179)

Into a 8-mL sealed tube purged and maintained with an inert atmosphere of nitrogen, was placed (3S,4R)-3-fluoro-N-(2-iodo-3-(2,2,2-trifluoroethyl)benzo[b]thiophen-7-yl)-1-(tetrahydro-2H-pyran-4-yl)piperidin-4-amine (0.100 g, 0.18 mmol), (6-methoxy-5-(prop-2-yn-1-ylamino)pyridin-2-yl)dimethylphosphine oxide(0. 060 g, 0.25 mmol), Pd(dppf)C12 (0.018 g, 0.03 mmol), CuI (0.012 g, 0.06 mmol), DIEA (0.078 g, 0.60 mmol), DMSO (1 mL). The reaction mixture was stirred at 50° C. for 1 h. The mixture was purified by preparative HPLC (Mobile Phase A: water (10 mmoL/L ammonium hydroxide), Mobile Phase B: MeCN; Flow rate: 70 mL/min; Gradient: 35-70-70% B (2-32-60 min) 264 nm; RT: 29.07-30.60 min) to afford (5-((3-(7-(((3S,4R)-3-fluoro-1-(tetrahydro-2H-pyran-4-yl)piperidin-4-yl)amino)-3-(2,2,2-trifluoroethyl)benzo[b]thiophen-2-yl)prop-2-yn-1-yl)amino)-6-methoxypyridin-2-yl)dimethylphosphine oxide (179) (0.0435 g, 36.15% yield) as white solid. LCMS: m/z=653 [M+1]⁺

¹H NMR (400 MHz, CD₃OD) δ 7.51 (t, J=6.8 Hz, 1H), 7.31 (t, J=7.8 Hz, 1H), 7.21 (d, J=7.8 Hz, 1H), 7.10 (d, J=7.2 Hz, 1H), 6.80 (d, J=7.6 Hz, 1H), 4.96-4.90 (m, 1H), 4.39 (s, 2H), 4.02 (d, J=11.8 Hz, 4H), 4.00 (s, 1H), 3.81-3.63 (m, 3H), 3.48-3.37 (m, 2H), 3.11-3.02 (m, 1H), 2.66-2.52 (m, 2H), 2.52-2.35 (m, 2H), 2.02-1.90 (m, 2H), 1.89-1.78 (m, 2H), 1.77 (s, 3H), 1.73 (s, 3H), 1.66-1.52 (m, 2H).

### Example 180

### (4-((3-(7-(((3S,4R)-1-(8-oxabicyclo[3.2.1]octan-3-yl)-3-fluoropiperidin-4-yl)amino)-3-(2,2,2-trifluoroethyl)benzo[b]thiophen-2-yl)prop-2-yn-1-yl)amino)-3-methoxyphenyl)dimethylphosphine oxide (180)

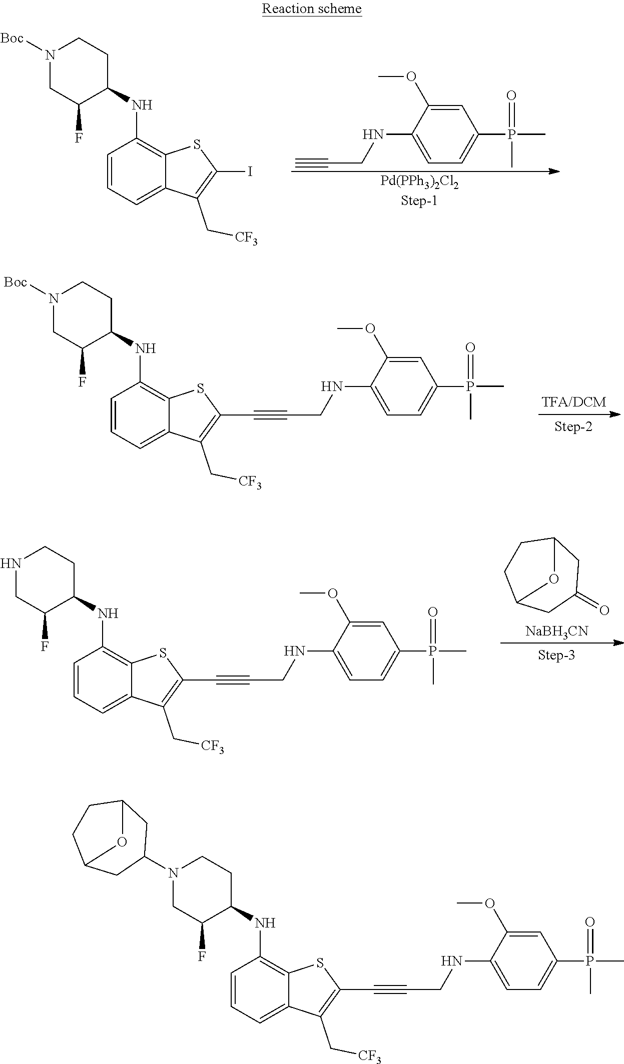

### Experiment Details

### Step 1. tert-butyl (3S,4R)-4-((2-(3-((4-(dimethylphosphoryl)-2-methoxyphenyl)amino)prop-1-yn-1-yl)-3-(2,2,2-trifluoroethyl)benzo[b]thiophen-7-yl)amino)-3-fluoropiperidine-1-carboxylate

Into a 4 mL vial purged and maintained with nitrogen atmosphere was placed tert-butyl (3S,4R)-3-fluoro-4-((2-iodo-3-(2,2,2-trifluoroethyl)benzo[b]thiophen-7-yl)amino)piperidine-1-carboxylate (0.836 g, 1.50 mmol), di methyl(7-(prop-2-yn-1-ylamino)-2,3-dihydrobenzofuran-4-yl)phosphine oxide (0.509 g, 2.15 mmol), Pd(PPh₃)₂ Cl₂ (0.098 g, 138.82 μmol), CuI (0.032 g, 168.02 μmol), TEA (0.283 g, 2.780 mmol), DMF (4 mL). The reaction was stirred for 1 h at room temperature. The reaction was diluted with water (4 mL), extracted with EA (4 mL×2). The combined organic layers were washed with water (4 mL) and brine (4 mL) successively, separated, then concentrated with vacuum. The residue was purified with C₁₈ column, eluted with ACN/water (v/v=3/2) to afford (7-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-2,3-dihydrobenzofuran-4-yl)dimethylphosphine oxide (0.450 g, 673.96 μmol, 38.07% yield) as light yellow solid. LCMS: m/z=668 [M+1]⁺. Hz, 6H).

### Step 2. (4-((3-(7-(((3S,4R)-3-fluoropiperidin-4-yl)amino)-3-(2,2,2-trifluoroethyl)benzo[b]thiophen-2-yl)prop-2-yn-1-yl)amino)-3-methoxyphenyl)dimethylphosphine oxide

Into a 50 mL flask was placed tert-butyl tert-butyl (3S,4R)-4-((2-(3-((4-(dimethylphosphoryl)-2-methoxyphenyl)amino)prop-1-yn-1-yl)-3-(2,2,2-trifluoroethyl)benzo[b]thiophen-7-yl)amino)-3-fluoropiperidine-1-carboxylate (0.450 g, 673.96 μmol), TFA (2 mL), DCM (6 mL). The reaction was stirred overnight at room temperature. The reaction quenched with saturated NaHCO₃(aq.), The solution was diluted with water (10 mL), extracted with EA (5 mL×3). The combined organic layers were washed with water (5 mL) and brine (5 mL) successively, separated, then concentrated with vacuum. The residue was purified with C₁₈ column, eluted with ACN/water (0.1% TFA) (v/v=1/3) to afford (4-((3-(7-(((3S,4R)-3-fluoropiperidin-4-yl)amino)-3-(2,2,2-trifluoroethyl)benzo[b]thiophen-2-yl)prop-2-yn-1-yl)amino)-3-methoxyphenyl)dimethylphosphine oxide (0.233 g, 410.52 μmol, 60.91% yield) as brown oil. LCMS: m/z=568[M+1]⁺

### Step 3. (4-((3-(7-(((3S,4R)-1-(8-oxabicyclo[3.2.1]octan-3-yl)-3-fluoropiperidin-4-yl)amino)-3-(2,2,2-trifluoroethyl)benzo[b]thiophen-2-yl)prop-2-yn-1-yl)amino)-3-methoxyphenyl)dimethylphosphine oxide (180)

Into a 8 mL flask was placed (4-((3-(7-(((3S,4R)-3-fluoropiperidin-4-yl)amino)-3-(2,2,2-trifluoroethyl)benzo[b]thiophen-2-yl)prop-2-yn-1-yl)amino)-3-methoxyphenyl)dimethylphosphine oxide (0.077 g, 135.66 μmol), 8-Oxabicyclo[3.2.1]octan-3-one (0.036 g, 285.37 μmol), Sodium cyanoborohydride (0.045 g, 1.05 mmol), acetic acid (0.01 μmol), Ethanol (1 mL) and stirred overnight at 50a. The reaction quenched with water (2 mL) and purified with prep-HPLC (Mobile Phase A: water (ammonium hydroxide), Mobile Phase B: ACN; Flow rate: 70 mL/min; Gradient: 50-82-85% B (2-30-36 min); 266 nm; RT: 34.15-35.80) to afford 4-[3-[4-[(1-methyl-4-piperidyl)amino]-1-(2,2,2-trifluoroethyl)indol-6-yl]prop-2-ynylamino]benzenesulfonamide (180) (27 mg, 51.96 μmol, 61.36% yield) as off-white solid. LCMS: m/z=678[M+1]⁺.

¹H NMR (400 MHz, DMSO-d₆) δ 7.30-7.09 (m, 4H), 6.87-6.82 (m, 1H), 6.77 (d, J=7.7 Hz, 1H), 6.07 (t, J=6.4 Hz, 1H), 5.12 (d, J=8.5 Hz, 1H), 4.79 (d, J=49.7 Hz, 1H), 4.34 (d, J=6.0 Hz, 4H) 3.85 (s, 3H), 3.78 (t, J=11.1 Hz, 2H), 3.64 (d, J=29.4 Hz, 1H), 3.10 (t, J=11.4 Hz, 1H), 2.87 (d, J=11.1 Hz, 1H), 2.77-2.65 (m, 1H), 2.37 (d, J=12.7 Hz, 1H), 2.24 (t, J=11.3 Hz, 1H), 1.90-1.63 (m, 6H), 1.62-1.43 (m, 10H).

### Example 181

### (4-((3-(7-(((3S,4R)-3-fluoro-1-(2,2,6,6-tetramethyltetrahydro-2H-pyran-4-yl)piperidin-4-yl)amino)-3-(2,2,2-tri fluoroethyl)benzo[b]thiophen-2-yl)prop-2-yn-1-yl)amino)-3-methoxyphenyl)dimethylphosphine oxide (181)

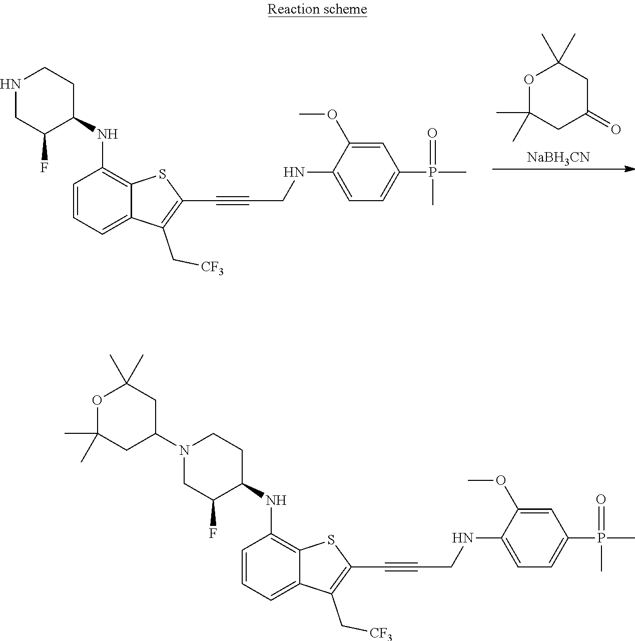

### Experiment Details

### Step 1. (4-((3-(7-(((3S,4R)-3-fluoro-1-(2,2,6,6-tetramethyltetrahydro-2H-pyran-4-yl)piperidin-4-yl)amino)-3-(2,2,2-trifluoroethyl)benzo[b]thiophen-2-yl)prop-2-yn-1-yl)amino)-3-methoxyphenyl)dimethylphosphine oxide (181)

Into a 8 mL flask was placed (4-((3-(7-(((3S,4R)-3-fluoropiperidin-4-yl)amino)-3-(2,2,2-trifluoroethyl)benzo[b]thiophen-2-yl)prop-2-yn-1-yl)amino)-3-methoxyphenyl)dimethylphosphine oxide (0.076 g, 133.90 μmol), 2,2,6,6-tetramethyl-2H-3,5,6-trihydropyran-4-one (0.032 g, 204.84 μmol), Sodium cyanoborohydride (0.041 g, 0.96 mmol), acetic acid (0.01 μmol), Ethanol (1 mL) and stirred overnight at 50a. The reaction quenched with water (2 mL) and purified with prep-HPLC (Mobile Phase A: water (ammonium hydroxide), Mobile Phase B: ACN; Flow rate: 70 mL/min; Gradient: 55-85-91% B (2-30-39 min); 266 nm; RT: 35.56-37.11) to afford (4-((3-(7-(((3S,4R)-3-fluoro-1-(2,2,6,6-tetramethyltetrahydro-2H-pyran-4-yl)piperidin-4-yl)amino)-3-(2,2,2-trifluoroethyl)benzo[b]thiophen-2-yl)prop-2-yn-1-yl)amino)-3-methoxyphenyl)dimethylphosphine oxide (181). LCMS: m/z=708 [M+1]⁺.

¹H NMR (400 MHz, DMSO-d₆) δ 7.29-7.09 (m, 4H), 6.84 (dd, J=8.1, 3.0 Hz, 1H), 6.77 (d, J=7.7 Hz, 1H), 6.08 (t, J=6.4 Hz, 1H), 5.15 (d, J=8.6 Hz, 1H), 4.81 (d, J=49.7 Hz, 1H), 4.34 (d, J=6.3 Hz, 2H), 3.89-3.75 (m, 5H), 3.66 (d, J=29.0 Hz, 1H), 3.12 (t, J=11.2 Hz, 1H), 2.98-2.84 (m, 2H), 2.57 (d, J=12.6 Hz, 1H), 2.36 (t, J=11.0 Hz, 1H), 1.88 (q, J=11.5 Hz, 1H), 1.74 (d, J=12.8 Hz, 1H), 1.58 (d, J=13.2 Hz, 8H), 1.20 (s, 8H), 1.12 (s, 6H).

### Example 182

### 5-((3-(7-((3-fluoro-1-methylpiperidin-4-yl)amino)-3-(2,2,2-trifluoroethyl)benzo[b]thiophen-2-yl)prop-2-yn-1-yl)amino)-6-methoxy-N-methylpicolinamide (182)

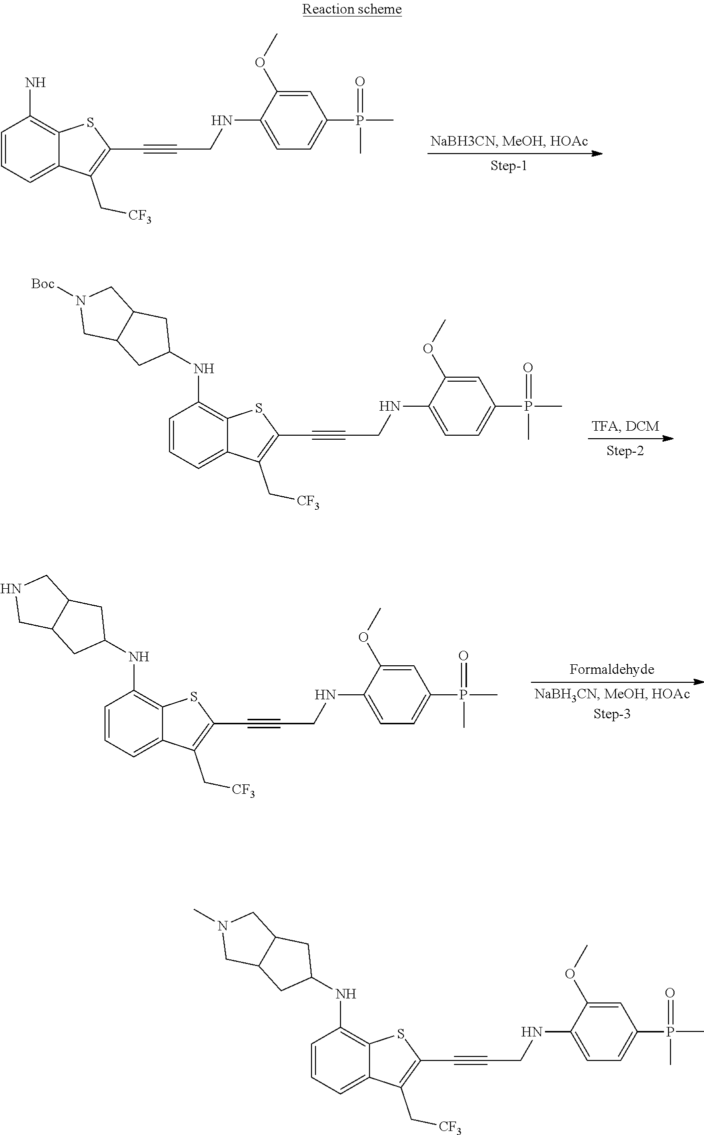

### Experiment Details

### Step 1. tert-butyl 5-((2-(3-((4-(dimethylphosphoryl)-2-methoxyphenyl)amino)prop-1-yn-1-yl)-3-(2,2,2-trifluoroethyl)benzo[b]thiophen-7-yl)amino)hexahydrocyclopenta[c]pyrrole-2(1H)-carboxylate

Into a 20-mL sealed tube was placed (4-((3-(7-amino-3-(2,2,2-trifluoroethyl)benzo[b]thiophen-2-yl)prop-2-yn-1-yl)amino)-3-methoxyphenyl)dimethylphosphine oxide (0.141 g, 0.30 mmol), tert-butyl 5-oxohexahydrocyclopenta[c]pyrrole-2(1H)-carboxylate (0.154 g, 0.68 mmol), MeOH (10 mL), HOAc (0.02 mL), NaBH₃CN (0.114 g, 2.66 mmol) was added after 30 min, the reaction mixture was stirred at 50° C. for 1 h. The mixture w as purified with C18 column eluted with ACN/water (v/v=2/1) to afford 0.131 g (64.13%) of tert-butyl5-((2-(3-((4-(dimethylphosphoryl)-2-methoxyphenyl)amino)prop-1-yn-1-yl)-3-(2,2,2-trifluoroethyl)benzo[b]thiophen-7-yl)amino)hexahydrocyclopenta[c]pyrrole-2(1H)-carboxylate as yellow oil. LCMS: m/z=676 [M+1]⁺.

### Step 2. (3-methoxy-4-((3-(7-((octahydrocyclopenta[c]pyrrol-5-yl)amino)-3-(2,2,2-trifluoroethyl)benzo[b]thiophen-2-yl)prop-2-yn-1-yl)amino)phenyl)dimethylphosphine oxide

Into a 20-mL sealed was placed tert-butyl5-((2-(3-((4-(dimethylphosphoryl)-2-methoxyphenyl)amino)prop-1-yn-1-yl)-3-(2,2,2-trifluoroethyl)benzo[b]thiophen-7-yl)amino)hexahydrocyclopenta[c]pyrrole-2(1H)-carboxylate (0.128 g, 0.19 mmol), TFA (0.5 mL), DCM (2 mL). The reaction mixture was stirred at room temperature for 1 h. The mixture was adjusted to pH 9 with NaHCO₃(aq). The aqueous layer was extracted with ethyl acetate (50 mL×2), the organic layers combined and dried over anhydrous Na₂SO₄, filtered and concentrated under vacuum. This resulted in 0.127 g (crude) of (3-methoxy-4-((3-(7-((octahydrocyclopenta[c]pyrrol-5-yl)amino)-3-(2,2,2-trifluoroethyl)benzo[b]thiophen-2-yl)prop-2-yn-1-yl)amino)phenyl)dimethylphosphine oxide as oil. LCMS: m/z=576 [M+1]⁺.

### Step 3. 5-((3-(7-((3-fluoro-1-methylpiperidin-4-yl)amino)-3-(2,2,2-trifluoroethyl)benzo[b]thiophen-2-yl)prop-2-yn-1-yl)amino)-6-methoxy-N-methylpicolinamide (182)

Into a 20-mL sealed tube was placed (3-methoxy-4-((3-(7-((octahydrocyclopenta[c]pyrrol-5-yl)amino)-3-(2,2,2-trifluoroethyl)benzo[b]thiophen-2-yl)prop-2-yn-1-yl)amino)phenyl)dimethylphosphine oxide (0.149 g, 0.26 mmol), Formaldehyde (0.017 g, 0.57 mmol), MeOH (3 mL), HOAc (0.02 mL), NaBH₃CN (0.045 g, 1.05 mmol) was added after 30 min, the reaction mixture was stirred at room temperature for 1 h. The mixture was purified by preparative HPLC (Mobile Phase A: water (10 mmoL/L ammonium hydroxide), Mobile Phase B: MeCN; Flow rate: 70 mL/min; Gradient: 50-85-100% B (2-30-45 min) 266 nm; RT: 40.06-42.49 min) to afford 5-((3-(7-((3-fluoro-1-methylpiperidin-4-yl)amino)-3-(2,2,2-trifluoroethyl)benzo[b]thiophen-2-yl)prop-2-yn-1-yl)amino)-6-methoxy-N-methylpicolinamide (182) (0.0427 g, 27.98% yield) as white solid. LCMS: m/z=590 [M+1]⁺

¹H NMR (400 MHz, DMSO) δ 7.32-7.17 (m, 2H), 7.17-7.07 (m, 2H), 6.92-6.81 (m, 1H), 6.76 (d, J=9.6 Hz, 1H), 6.72-6.60 (m, 1H), 6.07 (t, J=6.2 Hz, 1H), 4.33 (d, J=6.2 Hz, 2H), 4.00-3.88 (m, 1H), 3.85 (s, 3H), 3.82-3.70 (m, 2H), 2.69-2.58 (m, 3H), 2.49-2.41 (m, 1H), 2.33 (d, J=12. 2 Hz, 2H), 2.26 (d, J=8.8 Hz, 1H), 2.17-2.08 (m, 3H), 1.83-1.68 (m, 1H), 1.59-1.48 (m, 6H), 1.52 (s, 1H), 1.31-1.12 (m, 1H).

### Example 183

### N-(4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)-N-methylmethanesulfonamide (183)

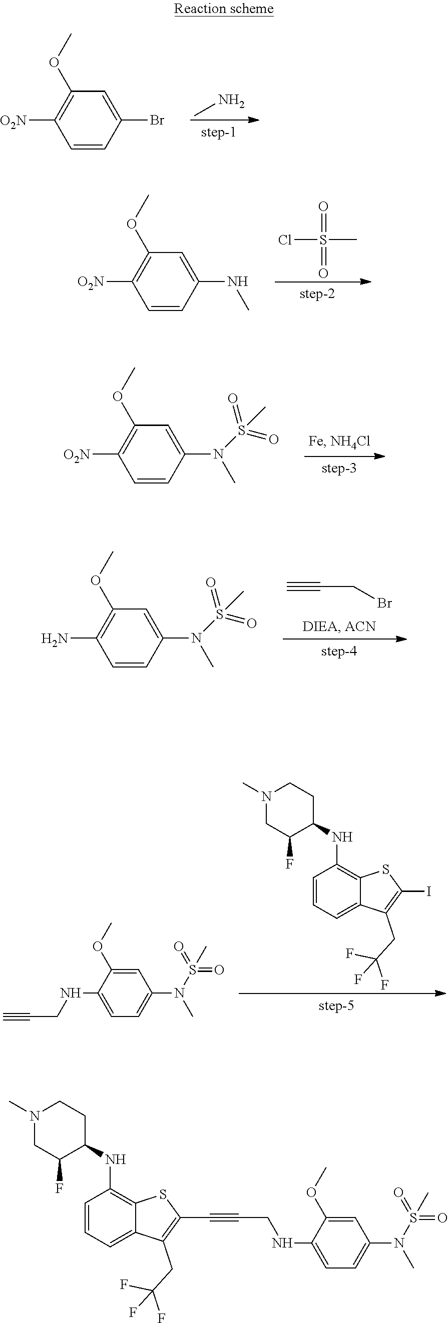

### Experimental Details

### Step 1. 3-methoxy-N-methyl-4-nitroaniline

Into a 500-mL three necked bottle purged and maintained with an inert atmosphere of nitrogen, was placed 4-bromo-2-methoxy-1-nitrobenzene (5.09 g, 21.93 mmol), Cu (620.00 g, 9.75 mmol), AcOH (3 mL), methanamine (30% in H₂O, 50 mL) and H₂O (50 mL). The reaction mixture was stirred at 100° C. for 3 h. The resulting solution was added to water (100 mL). The resulting solution was extracted with EA (2×150 mL). The organic layers combined and washed with brine (20 mL), dried over anhydrous Na₂SO₄, the residue was concentrated under vacuum. The residue was applied onto a silica gel column eluted with EA:hexane (v:v=1:1). This resulted in 3.37 g (84.32% yield) of 3-methoxy-N-methyl-4-nitroaniline as yellow solid. LCMS: m/z=183 [M+1]

### Step 2. N-(3-methoxy-4-nitrophenyl)-N-methylmethanesulfonamide

Into a 100-mL three necked bottle purged and maintained with an inert atmosphere of nitrogen, was placed 3-methoxy-N-methyl-4-nitroaniline (1.04 g, 5.70 mmol), TEA (1.66 g, 16.40 mmol) and DCM (20 mL). The reaction mixture was stirred at 0° C., then added MsCl (1.04 g, 9.04 mmol). The mixture was stirred at room temperature for 4 h. The resulting solution was quenched with water (50 mL). The resulting solution was extracted with EA (2×50 mL). The organic layers combined and washed with brine (50 mL), dried over anhydrous Na₂SO₄, the residue was concentrated under vacuum. The residue was applied onto a silica gel column eluted with EA:hexane (v:v=1:1). This resulted in 1.04 g (69.99% yield) of N-(3-methoxy-4-nitrophenyl)-N-methylmethanesulfonamide as yellow solid. LCMS: m/z=261 [M+1]⁺.

### Step 3. N-(4-amino-3-methoxyphenyl)-N-methylmethanesulfonamide

Into a 100-mL three necked bottle purged and maintained with an inert atmosphere of nitrogen, was placed N-(3-methoxy-4-nitrophenyl)-N-methylmethanesulfonamide (1.05 g, 4.03 mmol), Fe (1.03 g, 18.44 mmol), NH₄Cl (860.00 mg, 16.07 mmol), EtOH (20 mL) and H₂O (5 mL). The reaction mixture was stirred at 80° C. for 4 h. The resulting solution was filtered through a celite pad. The residue was concentrated under vacuum. The residue was applied onto a silica gel column eluted with EA:hexane (v:v=1:1). This resulted in 903.00 mg (97.19% yield) of N-(4-amino-3-methoxyphenyl)-N-methylmethanesulfonamide as yellow sol id. LCMS: m/z=231 [M+1]⁺

### Step 4. N-(3-methoxy-4-(prop-2-yn-1-ylamino)phenyl)-N-methylmethanesulfonamide

Into a 50-mL three necked bottle purged and maintained with an inert atmosphere of nitrogen, was placed N-(4-amino-3-methoxyphenyl)-N-methylmethanesulfonamide (1.02 g, 4.42 mmol), 3-bromoprop-1-yne (1.22 g, 10.25 mmol), DIEA (3.03 g, 23.44 mmol), MeCN (20 mL). The reaction mixture was stirred at 80° C. for 4 h. The residue was concentrated under vacuum. The residue was applied onto a C₁₈ column eluted with ACN/H₂O (0.01% TFA) (v:v=1:1), Flow rate: 100 m/min. This resulted in 520.00 mg (43.75% yield) of N-(3-methoxy-4-(prop-2-yn-1-ylamino)phenyl)-N-methylmethanesulfonamide as brown solid. LCMS: m/z=269 [M+1]⁺

### Step 5. N-(4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)-N-methylmethanesulfonamide (183)

Into a 50-mL three necked bottle purged and maintained with an inert atmosphere of nitrogen, was placed (3S,4R)-3-fluoro-N-(2-iodo-3-(2,2,2-trifluoroethyl)benzo[b]thiophen-7-yl)-1-methylpiperidin-4-amine (105.00 mg, 0.22 mmol), N-(3-methoxy-4-(prop-2-yn-1-ylamino)phenyl)-N-methylmethanesulfonamide (151.00 mg, 0.56 mmol), Pd(PPh₃)₂Cl₂ (26.00 mg, 0.036 mmol), CuI (52.00 mg, 0.27 mmol), DIEA (131.00 mg, 1.01 mmol), DMSO (8 mL). The reaction mixture was stirred at 50° C. for 3 h. The residue was applied onto a C₁₈ column eluted with ACN/H₂O (0.01% TFA) (v:v=1:1), Flow rate: 80 m/min. the residue was concentrated under vacuum. The resulting crude product was further purified by pre-HPLC with ACN/H₂O (0.1% ammonium hydroxide), Flow rate: 70 mL/min; Gradient: 35-70-80% B (2-30-40 min); 262 nm; RT: 35.52-37.07). This resulted in 32.00 mg (23.49% yield) of N-(4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)-N-methylmethanesulfonamide (183) as white solid. LCMS: m/z=613 [M+1]⁺

¹H NMR (400 MHz, DMSO-d₆) δ 7.33-7.16 (m, 2H), 7.01 (d, J=2.3 Hz, 1H), 6.90-6.85 (m, 1H), 6.80 (d, J=7.2 Hz, 1H), 6.69-6.65 (m, 1H), 5.13-5.10 (m, 1H), 5.02 (s, 2H), 4.80 (d, J=50.0 Hz, 1H), 4.11 (s, 2H), 3.95-3.86 (m, 2H), 3.66 (d, J=28.1 Hz, 1H), 3.14 (d, J=18.7 Hz, 3H), 3.08-3.00 (m, 1H), 2.82-2.73 (m, 6H), 2.32-2.15 (m, 4H), 2.13-2.05 (m, 1H), 2.00-1.87 (m, 1H), 1.75-1.68 (m, 1H).

### Example 184

### (4-((3-(7-(((3S,4R)-3-fluoro-1-(tetrahydro-2H-pyran-4-yl)piperidin-4-yl)amino)-3-(2,2,2-trifluoroethyl)benzo[b]thiophen-2-yl)prop-2-yn-1-yl)amino)-3-(methoxy-d₃)phenyl)dimethylphosphine oxide (184)

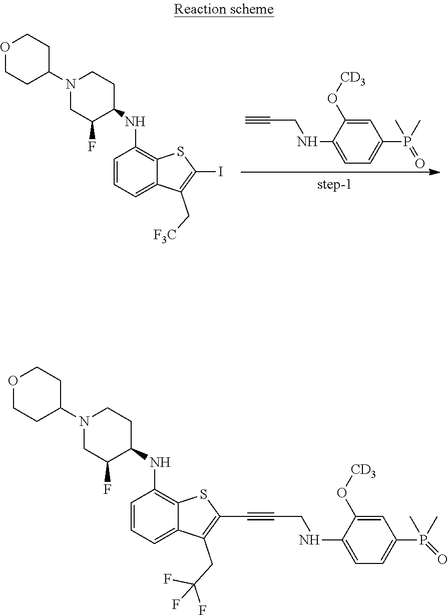 [SMILES: F[C@H]1CN(C2CCOCC2)CC[C@H]1Nc1cccc2c(CC(F)(F)F)c(I)sc12 ; [2H]C([2H])([2H])Oc1cc(P(C)(C)=O)ccc1NCC#C ; [2H]C([2H])([2H])Oc1cc(P(C)(C)=O)ccc1NCC#Cc1sc2c(N[C@@H]3CCN(C4CCOCC4)C[C@@H]3F)cccc2c1CC(F)(F)F]

### Experiment Details

### Step 1. (4-((3-(7-(((3S,4R)-3-fluoro-1-(tetrahydro-2H-pyran-4-yl)piperidin-4-yl)amino)-3-(2,2,2-trifluoroethyl)benzo[b]thiophen-2-yl)prop-2-yn-1-yl)amino)-3-(methoxy-d₃)phenyl)dimethylphosphine oxide (184)

Into a 8 mL vial was placed (3S,4R)-3-fluoro-N-(2-iodo-3-(2,2,2-trifluoroethyl)benzo[b]thiophen-7-yl)-1-(tetra hydro-2H-pyran-4-yl)piperidin-4-amine (0.07 g, 0.28 mmol), (3-(methoxy-d₃)-4-(prop-2-yn-1-ylamino)phenyl)di methylphosphine oxide (0.16 g, 0.30 mmol), bis(triphenylphosphine)palladium(II) chloride (0.037 g, 52.41 μmol), CuI (0.01 g, 52.51 μmol), DIEA (0.14 g, 1.07 mmol), Methyl sulfoxide (3 mL). The reaction was stirred at 60° C. for 1 h under nitrogen. The reaction was quenched with water (30 mL), extracted with EA (2×20 mL). The organic layers were combined, washed with brine (2×50 mL), dried over anhydrous s odium sulfate, filtered and concentrated under vacuum. The mixture was purified by preparative HPLC (Mobile Phase A: water (10 mmol/L ammonium hydroxide), Mobile Phase B: ACN; Flow rate: 70 mL/min; Gradient: 20-45-45% B (2-30-34 min); 266 nm; RT: 28.10-33.71 min) to provide the desired product. This resulted in 0.0475 g (24.59% yield) of (4-((3-(7-(((3S,4R)-3-fluoro-1-(tetrahydro-2H-pyran-4-yl)piperidin-4-yl) amino)-3-(2,2,2-trifluoroethyl)benzo[b]thiophen-2-yl)prop-2-yn-1-yl)amino)-3-(methoxy-d₃)phenyl)dimethylphosphine oxide (184) as off-white solid. LCMS: m/z=585 [M+1]⁺.

¹H NMR (400 MHz, DMSO-d₆) δ 7.24 (m, 8.3 Hz, 3H), 7.16-7.10 (m, 1H), 6.87-6.82 (m, 1H), 6.78 (d, J=7.7 Hz, 1H), 6.07 (t, J=6.4 Hz, 1H), 5.14 (d, J=8.5 Hz, 1H), 4.81 (d, J=49.7 Hz, 1H), 4.34 (d, J=6.4 Hz, 2H), 3.89 (d, J=9.8 Hz, 2H), 3.84-3.74 (m, 2H), 3.67 (d, J=29.3 Hz, 2H), 3.31-3.08 (m, 4H), 2.90 (d, J=11.1 Hz, 1H), 2.31 (t, J=10.8 Hz, 1H), 1.95-1.82 (m, 1H), 1.74 (d, J=12.9 Hz, 1H), 1.64 (d, J=12.5 Hz, 2H), 1.58 (d, J=13.2 Hz, 6H), 1.49-1.37 (m, 2H).

### Example 185

### (4-((3-(7-(((3S,4R)-3-fluoro-1-(oxepan-3-yl)piperidin-4-yl)amino)-3-(2,2,2-trifluoroethyl)benzo[b]thiophen-2-yl)prop-2-yn-1-yl)amino)-3-methoxyphenyl)dimethylphosphine oxide (185)

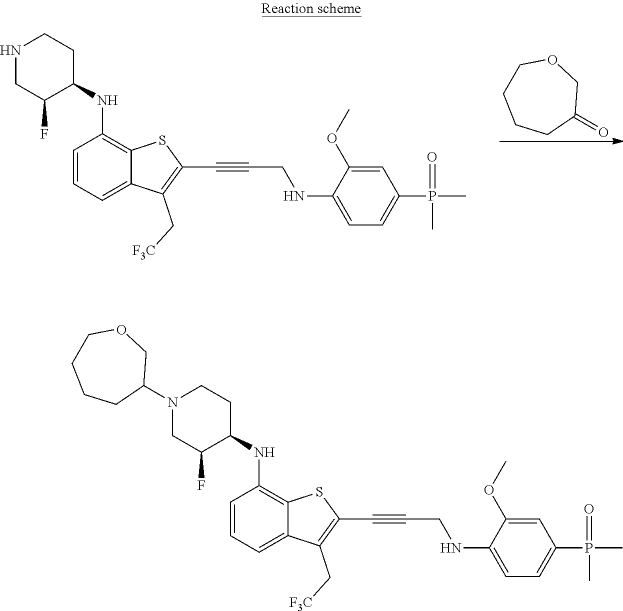 [SMILES: COc1cc(P(C)(C)=O)ccc1NCC#Cc1sc2c(N[C@@H]3CCNC[C@@H]3F)cccc2c1CC(F)(F)F ; O=C1CCCCOC1 ; COc1cc(P(C)(C)=O)ccc1NCC#Cc1sc2c(N[C@@H]3CCN(C4CCCCOC4)C[C@@H]3F)cccc2c1CC(F)(F)F]

### Experimental Details

### Step 1. (4-((3-(7-(((3S,4R)-3-fluoro-1-(oxepan-3-yl)piperidin-4-yl)amino)-3-(2,2,2-trifluoroethyl)benzo[b]thio phen-2-yl)prop-2-yn-1-yl)amino)-3-methoxyphenyl)dimethylphosphine oxide (185)

Into a 8-mL sealed tube purged and maintained with an inert atmosphere of nitrogen, was placed-oxepanone (0.078 g, 683.36 μmol), sodium cyanoboronhydride (0.038 g, 886.28 μmol), methanol (2 mL), acetic acid (0.1 mL), (4-((3-(7-(((3S,4R)-3-fluoropiperidin-4-yl)amino)-3-(2,2,2-trifluoroethyl)benzo[b]thiophen-2-yl)prop-2-yn-1-yl)amino)-3-methoxyphenyl)dimethylphosphine oxide (0.074 g, 130.38 μmol). The reaction mixture w as stirred at 50° C. for 18 h. The reaction was quenched by the addition of water (5 mL) and extracted with EA (2×5 mL). The organic layer was combined, washed with brine (5 mL), dried over anhydrous Na₂SO₄ and concentrated under vacuum. The mixture was purified by preparative HPLC (Mobile Phase A: water (10 mmoL/L ammonium hydroxide), Mobile Phase B: ACN; Flow rate: 70 mL/min; Gradient: 37-70-72% B (2-30-33 min; 266 nm; RT: 31.28-33.25 min). This resulted in 0.053 g (61.06% yield) of (4-((3-(7-(((3S,4R)-3-fluoro-1-(oxepan-3-yl)piperidin-4-yl)amino)-3-(2,2,2-trifluoroethyl)benzo[b]thiophen-2-yl)prop-2-yn-1-yl)amino)-3-methoxyphenyl)dimethylphosphine oxide (185) as white solid. LCMS: m/z=666 [M+1]⁺.

¹H NMR (400 MHz, DMSO) δ 7.26 (t, J=7.8 Hz, 1H), 7.20 (d, J=8.1 Hz, 2H), 7.17-7.11 (m, 1H), 6.88-6.82 (m, 1H), 6.77 (d, J=7.6 Hz, 1H), 6.07 (t, J=6.3 Hz, 1H), 5.13 (d, J=8.5 Hz, 1H), 4.8 8-4.71 (m, 1H), 4.33 (d, J=6.2 Hz, 2H), 3.85 (s, 3H), 3.82-3.72 (m, 2H), 3.70-3.58 (m, 3H), 3.54-3.44 (m, 2H), 3.06-2.97 (m, 1H), 2.77 (s, 1H), 2.70-2.56 (m, 2H), 2.44-2.30 (m, 2H), 1.94-1.84 (m, 1H), 1.81-1.67 (m, 4H), 1.59 (s, 3H), 1.57-1.50 (m, 5H).

### Example 186

### N-(4-(dimethylphosphoryl)-2-methoxyphenyl)-3-(7-(((3S,4R)-3-fluoro-1-methylpiperidin-4-yl)amino)-3-(2,2,2-trifluoroethyl)benzo[b]thiophen-2-yl)propiolamide (186)

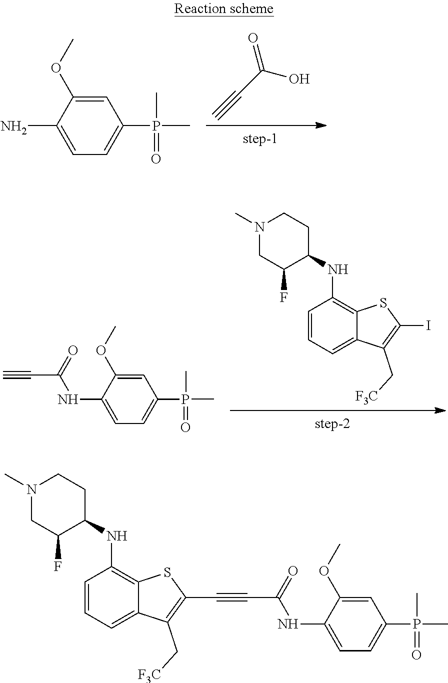

### Experiment Details

### Step 1. N-(4-(dimethylphosphoryl)-2-methoxyphenyl)propiolamide

Into a 100 mL round-bottom flask was placed propiolic acid (1.28 g, 18.32 mmol), DCM (50 mL) and stirred at 0° C. DCC (3.42 g, 16.58 mmol) was added to the mixture slowly at 0° C. and stirred at 0° C. for 2 h. (4-amino-3-methoxyphenyl)dimethylphosphine oxide (0.30 g, 1.50 mmol), N-(4-pyridyl)dimethylamine (0.18 g, 1.51 mmol) were added to the mixture and stirred for 1 h. The reaction was quenched with brine (200 mL), extracted with DCM (2×100 mL). The organic layers were dried over anhydrous sodium sulfate, filtered and concentrated under vacuum. The residue purified by flash-C18 eluted with ACN/water (v/v=1/9) to provide the desired product. This resulted in 0.301 g (79.82% yield) of N-(4-(dimethylphosphoryl)-2-m ethoxyphenyl)propiolamide as brown solid. LCMS: m/z=252 [M+1]⁺.

### Step 2. N-(4-(dimethylphosphoryl)-2-methoxyphenyl)-3-(7-(((3S,4R)-3-fluoro-1-methylpiperidin-4-yl)amino)-3-(2,2,2-trifluoroethyl)benzo[b]thiophen-2-yl)propiolamide (186)

Into a 20 mL vial was placed (3S,4R)-3-fluoro-N-(2-iodo-3-(2,2,2-trifluoroethyl)benzo[b]thiophen-7-yl)-1-meth ylpiperidin-4-amine (0.12 g, 0.26 mmol), N-(4-(dimethylphosphoryl)-2-methoxyphenyl)propiolamide (0.20 g, 0.81 mmol), bis(triphenylphosphine)palladium(II) chloride (0.06 g, 82.16 μmol), CuI (0.02 g, 94.51 μmol), DIEA (0.17 g, 1.34 mmol), Methyl sulfoxide (5 mL). The reaction was stirred at 60° C. for 1 h under nitro gen. The reaction was quenched with water (30 mL), extracted with EA (2×20 mL). The organic layers were combined, washed with brine (2×50 mL), dried over anhydrous sodium sulfate, filtered and concentrated under vacuum. The mixture was purified by preparative HPLC (Mobile Phase A: water (10 mmol/L NH₄0H), Mobile Phase B: ACN; Flow rate: 70 mL/min; Gradient: 35-68-68% B (2-30-32 min); 262 nm; RT: 29.06-30.92 min) to provide the desired product. This resulted in 0.0349 g (22.50% yield) of N-(4-(dimethylphosphoryl)-2-methoxyphenyl)-3-(7-(((3S,4R)-3-fluoro-1-methylpiperidin-4-yl)amino)-3-(2,2,2-trifluoroethyl)benzo[b]thiophen-2-yl)propiolamide (186) as yellow solid. LCMS: m/z=596 [M+1]⁺.

¹H NMR (400 MHz, DMSO-d₆) δ 10.46 (s, 1H), 7.96 (d, J=7.9 Hz, 1H), 7.47-7.30 (m, 4H), 6.88 (t, J=4.7 Hz, 1H), 5.36 (d, J=8.4 Hz, 1H), 4.83 (d, J=49.4 Hz, 1H), 4.25-4.12 (m, 2H), 3.94 (s, 3H), 3.69 (d, J=29.6 Hz, 1H), 3.05 (t, J=11.4 Hz, 1H), 2.82 (d, J=10.8 Hz, 1H), 2.33-2.16 (m, 4H), 2.15-1.93 (m, 2H), 1.75 (d, J=12.6 Hz, 1H), 1.67 (d, J=13.3 Hz, 6H).

### Example 187

### (7-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-2,3-dihydrobenzofuran-4-yl)dimethylphosphine oxide (187)

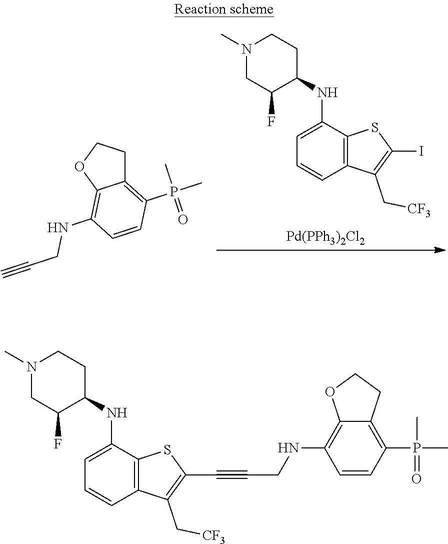

### Experiment Details

### Step 1. (7-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-2,3-dihydrobenzofuran-4-yl)dimethylphosphine oxide (187)

Into a 4 mL vial purged and maintained with nitrogen atmosphere was placed (3S,4R)-3-fluoro-N-(2-iodo-3-(2,2,2-trifluoroethyl)benzo[b]thiophen-7-yl)-1-methylpiperidin-4-amine (0.129 g, 517.56 μmol), dimethyl(7-(prop-2-yn-1-ylamino)-2,3-dihydrobenzofuran-4-yl)phosphine oxide (0.107 g, 226.56 μmol), Pd(PPh₃)₂Cl₂ (0.018 g, 25.50 μmol), CuI (0.014 g, 73.51 μmol), TEA (0.043 g, 424.95 μmol), DMF (1 mL), and stirred overnight at 50 gh The reaction was diluted with water (2 mL), extracted with EA (2 mL×2). The combined organic layers were washed with water (2 mL) and brine (2 mL) successively, separated, then concentrated with vacuum. The residue was purified with prep-HPLC (Mobile Phase A: water (NH₄OH), Mobile Phase B: ACN; Flow rate: 70 mL/min; Gradient: 35-70-70% B (2-30-32 min); 266 nm; RT: 28.87-31.37) to afford (7-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-2,3-dihydrobenzofuran-4-yl)dimethylphosphine oxide (187) (29 mg, 48.85 μmol, 13.59% yield) as white solid. LCMS: m/z=594 [M+1]⁺.

¹H NMR (400 MHz, DMSO-d₆) δ 7.33-7.17 (m, 2H), 7.09-6.98 (m, 1H), 6.81-6.67 (m, 2H), 5.99 (t, J=6.4 Hz, 1H), 5.19 (d, J=8.5 Hz, 1H), 4.80 (d, J=49.5 Hz, 1H), 4.56 (t, J=8.8 Hz, 2H), 4.31 (d, J=6.4 Hz, 2H), 3.79 (q, J=11.0 Hz, 2H), 3.64 (d, J=29.0 Hz, 1H), 3.39 (s, 2H), 3.03 (t, J=11.3 Hz, 1H), 2.79 (d, J=11.2 Hz, 1H), 2.26 (d, J=13.1 Hz, 1H), 2.18 (s, 3H), 2.07 (t, J=11.5 Hz, 1H), 2.01-1.89 (m, 1H), 1.71 (d, J=12.6 Hz, 1H), 1.58 (d, J=13.1 Hz, 6H).

### Example 188

### (4-((3-(7-((3-fluoro-1-methylpiperidin-4-yl)amino)-3-(2,2,2-trifluoro-1-hydroxyethyl)benzo[b]thiophen-2-yl)prop-2-yn-1-yl)amino)-3-methoxyphenyl)dimethylphosphine oxide(racemic) (188)

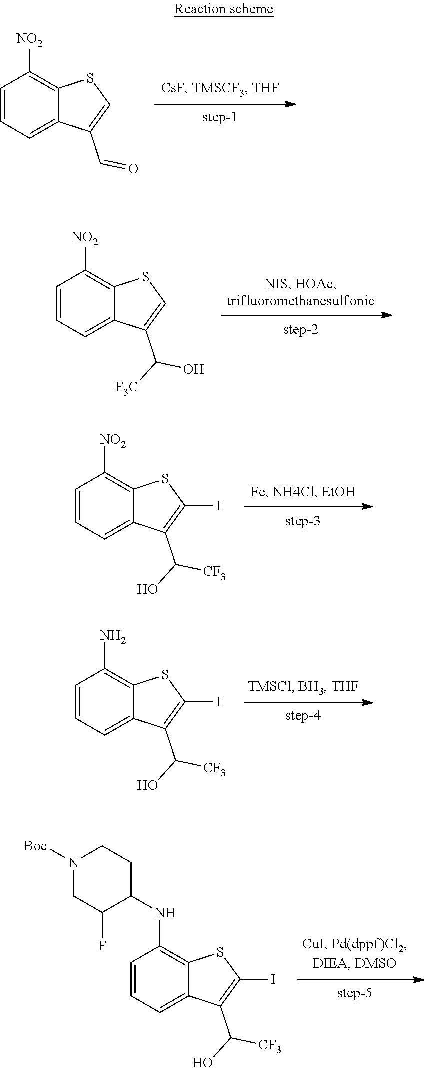 [SMILES: O=Cc1csc2c([N+](=O)[O-])cccc12 ; O=[N+]([O-])c1cccc2c(C(O)C(F)(F)F)csc12 ; O=[N+]([O-])c1cccc2c(C(O)C(F)(F)F)c(I)sc12 ; Nc1cccc2c(C(O)C(F)(F)F)c(I)sc12 ; CC(C)(C)OC(=O)N1CCC(Nc2cccc3c(C(O)C(F)(F)F)c(I)sc23)C(F)C1]

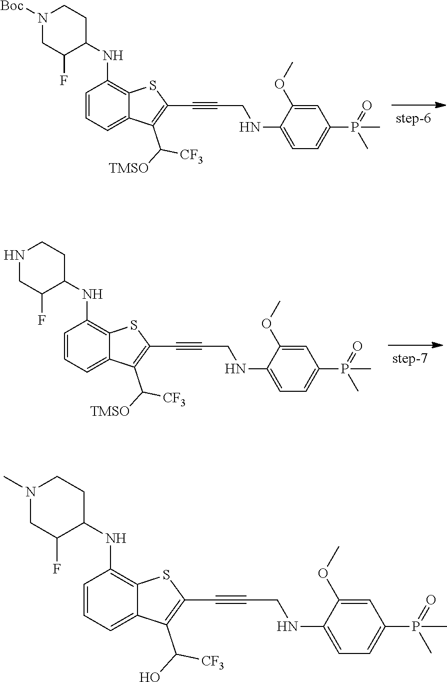

### Experiment Details

### Step 1. 2,2,2-trifluoro-1-(7-nitrobenzo[b]thiophen-3-yl)ethan-1-ol (racemic)

Into a 20-mL sealed tube was placed 7-nitrobenzo[b]thiophene-3-carbaldehyde (0.200 g, 0.97 mmol), cesium fluoride (0.495 g, 3.26 mmol), THF (3 mL), (Trifluoromethyl)trimethylsilane (0.478 g, 3.36 mmol). The reaction mixture was stirred at room temperature for 0.5 h. The mixture extracted with EA/H₂O (50 mL×2). The combined organic layers were washed with brine (30 mL), dried over anhydrous Na₂SO₄ and concentrated under vacuum. The residue was applied onto a silica gel column eluted with EA/hexane (v/v=1/9). This resulted in 0.150 g (56.06% yield) of 2,2,2-trifluoro-1-(7-nitrobenzo[b]thiophen-3-yl)ethan-1-ol (race mic) as yellow solid.

### Step 2. 2,2,2-trifluoro-1-(2-iodo-7-nitrobenzo[b]thiophen-3-yl)ethan-1-ol (racemic)

Into a 20-mL sealed tube was placed 2,2,2-trifluoro-1-(7-nitrobenzo[b]thiophen-3-yl)ethan-1-ol (racemic) (0.424 g, 1.53 mmol), HOAc (5 mL), Trifluoromethanesulfonic acid (1 mL). The reaction mixture was stirred at room temperature for 12 h. The mixture extracted with EA/H₂O (50 mL×2). The combined organic layers were washed with brine (30 mL), dried over anhydrous Na₂SO₄ and concentrated under vacuum. The residue was applied onto a silica gel column eluted with EA/hexane (v/v=1/10). This resulted in 0.401 g (65.04% yield) of 2,2,2-trifluoro-1-(2-iodo-7-nitrobenzo[b]thiophen-3-yl)ethan-1-ol (racemic) as yellow solid.

### Step 3. 1-(7-amino-2-iodobenzo[b]thiophen-3-yl)-2,2,2-trifluoroethan-1-ol(racemic)

Into a 20-mL sealed tube was placed 2,2,2-trifluoro-1-(2-iodo-7-nitrobenzo[b]thiophen-3-yl)ethan-1-ol (racemic) (0.392 g, 0.97 mmol), Iron (0.575 g, 10.30 mmol), NH₄Cl (0.642 g, 12.00 mmol), EtOH (5 mL) and H₂O (1 mL). The reaction mixture was stirred at 70° C. for 1 h. The mixture extracted with EA/H₂O (50 mL×2). The combined organic layers were washed with brine (30 mL), dried over anhydrous Na₂SO₄ and concentrated under vacuum. The residue was applied onto a silica gel column eluted with EA/hexane (v/v=1/3). This resulted in 0.232 g (63.94% yield) of 1-(7-amino-2-iodobenzo[b]thiophen-3-yl)-2,2,2-trifluoroethan-1-ol(racemic) as yellow solid. LCMS: m/z=374 [M+1]⁺.

### Step 4. tert-butyl 3-fluoro-4-((2-iodo-3-(2,2,2-trifluoro-1-hydroxyethyl)benzo[b]thiophen-7-yl)amino)piperidine-1-carboxylate (racemic)

A mixture of 1-(7-amino-2-iodobenzo[b]thiophen-3-yl)-2,2,2-trifluoroethan-1-ol(racemic) (0.188 g, 0.50 mmol), tert-butyl 3-fluoro-4-oxopiperidine-1-carboxylate (0.192 g, 0.88 mmol), and TMSCl (1 mL) in DMF (4 mL) was degassed and purged with N₂(g), and then BH₃·g), and th2 mL) was added. The mixture was stirred at 0° C. for 1 h under N₂ atmosphere. The reaction mixture was quenched with water (20 mL) at 0° C. and extracted with EA (40 mL×2). The combined organic layers were washed with brine (30 mL), dried over anhydrous sodium sulfate, filtered, and concentrated under reduced pressure. The residue was purified by silica gel column, eluted with EA/hexane (v/v=1/5) to afford tert-butyl 3-fluoro-4-((2-iodo-3-(2,2,2-trifluoro-1-hydroxyethyl)benzo[b]thiophen-7-yl)amino)piperidine-1-carboxylate (racemic) (0.046 g, 15.90% yield) as oil. LCMS: m/z=575 [M+1]⁺

### Step 5. tert-butyl 4-((2-(3-((4-(dimethylphosphoryl)-2-methoxyphenyl)amino)prop-1-yn-1-yl)-3-(2,2,2-trifluoro-1-((trimethylsilyl)oxy)ethyl)benzo[b]thiophen-7-yl)amino)-3-fluoropiperidine-1-carboxylate (racemic)

Into a 8-mL sealed tube purged and maintained with an inert atmosphere of nitrogen, was placed tert-butyl 3-fluoro-4-((2-iodo-3-(2,2,2-trifluoro-1-hydroxyethyl)benzo[b]thiophen-7-yl)amino)piperidine-1-carboxylate (race mic) (0.046 g, 0.08 mmol), (3-methoxy-4-(prop-2-yn-1-ylamino)phenyl)dimethylphosphine oxide (0.043 g, 0.18 mmol), Pd(dppf)Cl₂ (0.004 g, 0.01 mmol), CuI (0.006 g, 0.03 mmol), DIEA (0.032 g, 0.25 mmol), DMSO (1 mL). The mixture was stirred at 50° C. for 1 h. The mixture was purified with C18 column eluted with ACN/water (v/v=1/5) to afford 0.042 g (76.74%) of tert-butyl 4-((2-(3-((4-(dimethylphosphoryl)-2-methoxyphenyl)amino)prop-1-yn-1-yl)-3-(2,2,2-trifluoro-1-((trimethylsilyl)oxy)ethyl)benzo[b]thiophen-7-yl)amino)-3-fluoropiperidine-1-carboxylate (racemic) as oil. LCMS: m/z=756 [M+1]⁺

### Step 6. (4-((3-(7-((3-fluoropiperidin-4-yl)amino)-3-(2,2,2-trifluoro-1-((trimethylsilyl)oxy)ethyl)benzo[b]thiophen-2-yl)prop-2-yn-1-yl)amino)-3-methoxyphenyl)dimethylphosphine oxide(racemic)

Into a 8-mL sealed tube was placed tert-butyl 4-((2-(3-((4-(dimethylphosphoryl)-2-methoxyphenyl)amino)prop-1-yn-1-yl)-3-(2,2,2-trifluoro-1-((trimethylsilyl)oxy)ethyl)benzo[b]thiophen-7-yl)amino)-3-fluoropiperidine-1-carboxylate (racemic)(0.038 g, 0.06 mmol), TFA (0.3 mL), DCM (1 mL). The mixture was stirred at room temperature for 1 h. The mixture was adjusted to pH 9 with NaHCO₃(aq). The aqueous layer was extracted with ethyl acetate (50 mL×2), the organic layers combined and dried over anhydrous Na₂SO₄, filtered and concentrated under vacuum. This resulted in 0.034 g (crude) (4-((3-(7-((3-fluoropiperidin-4-yl)amino)-3-(2,2,2-trifluoro-1-((trimethylsilyl)oxy)ethyl)benzo[b]thiophen-2-yl)prop-2-yn-1-yl)amino)-3-methoxyphenyl)dimethyl phosphine oxide(racemic) as yellow oil. LCMS: m/z=656 [M+1]⁺

### Step 7. (4-((3-(7-((3-fluoro-1-methylpiperidin-4-yl)amino)-3-(2,2,2-trifluoro-1-hydroxyethyl)benzo[b]thiophen-2-yl)prop-2-yn-1-yl)amino)-3-methoxyphenyl)dimethylphosphine oxide(racemic) (188)

Into a 8-mL sealed tube was placed (4-((3-(7-((3-fluoropiperidin-4-yl)amino)-3-(2,2,2-trifluoro-1-((trimethylsilyl)oxy)ethyl)benzo[b]thiophen-2-yl)prop-2-yn-1-yl)amino)-3-methoxyphenyl)dimethylphosphine oxide(racemic) (0.032 g, 0.05 mmol), Formaldehyde (0.004 g, 0.06 mmol), MeOH (3 mL), HOAc (0.02 mL), NaBH₃CN (0.020 g, 0.47 mmol) was added after 30 min, the reaction mixture was stirred at room temperature for 1 h, TBAF (1M, 1 mL) was added. The mixture was purified by preparative HPLC (Mobile Phase A: water (10 mmoL/L ammonium hydroxide), Mobile Phase B: MeCN; Flow rate: 70 mL/min; Gradient: 50-85-100% B (2-30-45 min) 266 nm; RT: 35.06-36.49 min) to afford (4-((3-(7-((3-fluoro-1-methylpiperidin-4-yl)amino)-3-(2,2,2-trifluoro-1-hydroxyethyl)benzo[b]thiophen-2-yl)prop-2-yn-1-yl)amino)-3-methoxyphenyl)dimethylphosphine oxide(racemic) (188) (0.003 g, 10.07% yield) as white solid. LCMS: m/z=598 [M+1]⁺

### Example 189 (3-methoxy-4-((3-(7-((2-methyl-2-azaspiro[3.3]heptan-6-yl)amino)-3-(2,2,2-trifluoroethyl)benzo[b]thiophen-2-yl)prop-2-yn-1-yl)amino)phenyl)dimethylphosphine oxide (189)

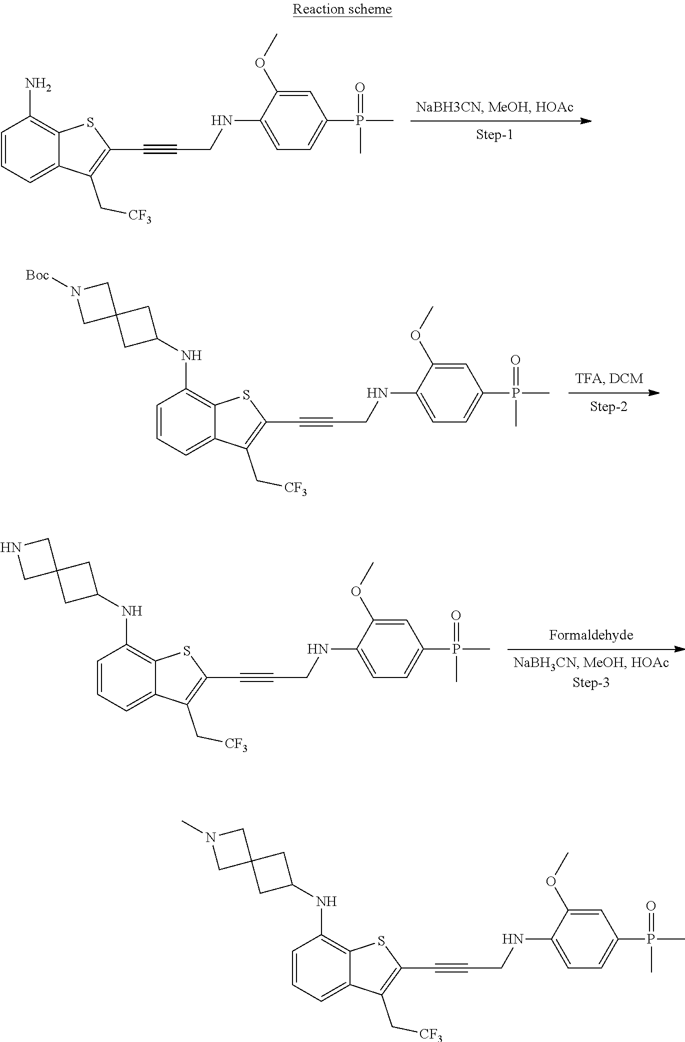

### Experiment Details

### Step 1. tert-butyl 6-((2-(3-((4-(dimethylphosphoryl)-2-methoxyphenyl)amino)prop-1-yn-1-yl)-3-(2,2,2-trifluoroethyl)benzo[b]thiophen-7-yl)amino)-2-azaspiro[3.3]heptane-2-carboxylate

Into a 20-mL sealed tube was placed (4-((3-(7-amino-3-(2,2,2-trifluoroethyl)benzo[b]thiophen-2-yl)prop-2-yn-1-yl)amino)-3-methoxyphenyl)dimethylphosphine oxide (0.198 g, 0.42 mmol), tert-butyl 6-oxo-2-azaspiro[3.3]heptane-2-carboxylate (0.216 g, 1.02 mmol), MeOH (5 mL), HOAc (0.02 mL), NaBH₃CN (0.150 g, 3.50 m mol) was added after 30 min, the reaction mixture was stirred at 50° C. for 12 h. The mixture was purified with C18 column eluted with ACN/water (v/v=2/1) to afford 0.160 g (59.96%) of tert-butyl 6-((2-(3-((4-(dimethylphosphoryl)-2-methoxyphenyl)amino)prop-1-yn-1-yl)-3-(2,2,2-trifluoroethyl)benzo[b]thiophen-7-yl)amino)-2-azaspiro[3.3]heptane-2-carboxylate as yellow oil. LCMS: m/z=662 [M+1]⁺.

### Step 2. (4-((3-(7-((2-azaspiro[3.3]heptan-6-yl)amino)-3-(2,2,2-trifluoroethyl)benzo[b]thiophen-2-yl)prop-2-yn-1-yl)amino)-3-methoxyphenyl)dimethylphosphine oxide

Into a 20-mL sealed was placed tert-butyl 6-((2-(3-((4-(dimethylphosphoryl)-2-methoxyphenyl)amino)prop-1-yn-1-yl)-3-(2,2,2-trifluoroethyl)benzo[b]thiophen-7-yl)amino)-2-azaspiro[3.3]heptane-2-carboxylate (0.127 g, 0.19 mmol), TFA (0.5 mL), DCM (2 mL). The reaction mixture was stirred at room temperature for 1 h. The mixture was adjusted to pH 9 with NaHCO₃(aq). The aqueous layer was extracted with ethyl acetate (50 mL×2), the organic layers was combined and dried over anhydrous Na₂SO₄, filtered and concentrated un der vacuum. This resulted in 0.127 g (crude) of (4-((3-(7-((2-azaspiro[3.3]heptan-6-yl)amino)-3-(2,2,2-trifluoroethyl)benzo[b]thiophen-2-yl)prop-2-yn-1-yl)amino)-3-methoxyphenyl)dimethylphosphine oxide as yellow oil. LCMS: m/z=562 [M+1]⁺.

### Step 3. (3-methoxy-4-((3-(7-((2-methyl-2-azaspiro[3.3]heptan-6-yl)amino)-3-(2,2,2-trifluoroethyl)benzo[b]thio phen-2-yl)prop-2-yn-1-yl)amino)phenyl)dimethylphosphine oxide (189)

Into a 20-mL sealed tube was placed (4-((3-(7-((2-azaspiro[3.3]heptan-6-yl)amino)-3-(2,2,2-trifluoroethyl)benzo[b]thiophen-2-yl)prop-2-yn-1-yl)amino)-3-methoxyphenyl)dimethylphosphine oxide (0.127 g, 0.26 mmol), Formaldehyde (0.008 g, 0.27 mmol), MeOH (5 mL), HOAc (0.02 mL), NaBH₃CN (0.058 g, 1.35 mmol) was added after 30 min, the reaction mixture was stirred at room temperature for 1 h. The mixture was purified by preparative HPLC (Mobile Phase A: water (10 mmoL/L ammonium hydroxide), Mobile Phase B: Me CN; Flow rate: 70 mL/min; Gradient: 50-85-100% B (2-30-45 min) 266 nm; RT: 40.06-42.49 min) to afford (3-methoxy-4-((3-(7-((2-methyl-2-azaspiro[3.3]heptan-6-yl)amino)-3-(2,2,2-trifluoroethyl)benzo[b]thiophen-2-yl) prop-2-yn-1-yl)amino)phenyl)dimethylphosphine oxide (189) (0.0481 g, 43.54% yield) as white solid. LCMS: m/z=576 [M+1]⁺

¹H NMR (400 MHz, DMSO) δ 7.28-7.18 (m, 2H), 7.18-7.06 (m, 2H), 6.90-6.80 (m, 1H), 6.47 (d, J=7.7 Hz, 1H), 6.07 (t, J=6.3 Hz, 1H), 5.83 (d, J=6.4 Hz, 1H), 4.33 (d, J=6.3 Hz, 2H), 3.85 (s, 3H), 3.84-3.65 (m, 3H), 3.21 (s, 2H), 3.09 (s, 2H), 2.60-2.53 (m, 1H), 2.17 (d, J=11.6 Hz, 3H), 2.11-1.99 (m, 2H), 1.60 (s, 3H), 1.56 (s, 3H), 1.23 (s, 1H).

### Example 192

4-(4-((3-(7-((3-fluoro-1-methylpiperidin-4-yl)amino)-3-(2,2,2-trifluoroethyl)benzo[b]thiophen-2-yl)prop-2-yn-1-yl)amino)-3-methoxyphenyl)morpholin-3-one (192)

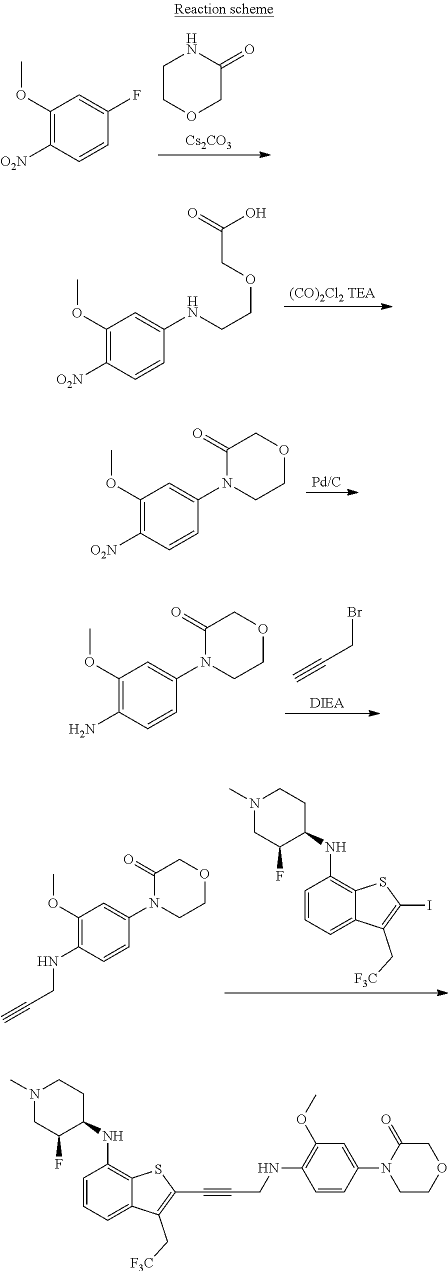

### Experiment Details

### Step 1. 2-(2-((3-methoxy-4-nitrophenyl)amino)ethoxy)acetic acid

Into an 100 mL flask was placed 4-fluoro-2-methoxy-1-nitrobenzene (2.030 g, 11.86 mmol), morpholin-3-one (1.423 g, 14.07 mmol), Cs₂CO₃ (5.693 g, 17.47 mmol), DMF (10 mL). The reaction was stirred for 6 h at 120° C. The reaction was neutralized with 5M HCl(aq.), diluted with water (100 mL), extracted with E A (50 mL×3). The combined organic layer was washed with water (100 mL) and concentrated to afford 2-[2-(3-methoxy-4-nitro-anilino)ethoxy]acetic acid (3.25 g, 12.03 mmol, crude) as yellow oil. LCMS: m/z=271[M+1]⁺.

Step 2. 4-(3-methoxy-4-nitrophenyl)morpholin-3-one Into an ice-cold 250 mL flask was placed 2-[2-(3-methoxy-4-nitro-anilino)ethoxy]acetic acid (3.312 g, 12.26 mmol), Oxalyl chloride (1.509 g, 11.89 mmol), DCM (10 mL), DMF (1 mL). The reaction was stirred for 2 h at Oxalyl c (1.419 g, 14.02 mmol) was added to the solution and stirred for additional 2 h. The reaction quenched with EtOH (5 mL) and concentrated. The residue purified with C₁₈ column, eluted with A CN/water (v/v=1/3) to give 4-(3-methoxy-4-nitrophenyl)morpholin-3-one (0.659 g, 2.61 mmol, 21.32% yield) as off-white solid. LCMS: m/z=253[M+1]⁺.

### Step 3. 4-(4-amino-3-methoxyphenyl)morpholin-3-one

Into a 50 mL flask was placed 4-(3-methoxy-4-nitrophenyl)morpholin-3-one (0.655 g, 2.60 mmol), Pd/C (0.350 g, 3.29 mmol), MeOH (3 mL). The reaction was stirred overnight at Hydrogen atmosphere, room temperature. The reaction was filtered, the filter cake was washed with MeOH (5 mL×2). The filtrate was concentrated to afford 4-(4-amino-3-methoxyphenyl)morpholin-3-one (0.088 g, 395.97 μmol, 15.25% yield) as off-white solid. LCMS: m/z=223[M+1]⁺

### Step 4. 4-(3-methoxy-4-(prop-2-yn-1-ylamino)phenyl)morpholin-3-one

Into a 50 mL flask was placed 4-(4-amino-3-methoxyphenyl)morpholin-3-one (0.105 g, 472.46 μmol), 3-bromoprop-1-yne (0.073 g, 613.65 μmol), DIEA (0.097 g, 750.53 μmol), DMF (1 mL). The reaction was stirred for 5 h at 50° C. The reaction was purified with C₁₈ column, eluted with ACN/water (v/v=1/3) to afford 4-(3-methoxy-4-(prop-2-yn-1-ylamino)phenyl)morpholin-3-one (0.031 g, 119.10 μmol, 25.21% yield) as light brown solid. LCMS: m/z=261[M+1]⁺

### Step 5. 4-(4-((3-(7-((3-fluoro-1-methylpiperidin-4-yl)amino)-3-(2,2,2-trifluoroethyl)benzo[b]thiophen-2-yl)prop-2-yn-1-yl)amino)-3-methoxyphenyl)morpholin-3-one (192)

4 mL vial purged and maintained with nitrogen atmosphere was placed (3S,4R)-3-fluoro-N-(2-iodo-3-(2,2,2-trifluoroethyl)benzo[b]thiophen-7-yl)-1-methylpiperidin-4-amine (0.057 g, 120.69 μmol), 4-(3-methoxy-4-(prop-2-yn-1-ylamino)phenyl)morpholin-3-one (0.028 g, 107.57 μmol), Pd(PPh₃)₂Cl₂ (0.012 g, 17.00 μmol), CuI (0.011 g, 57.76 μmol), TEA (0.039 g, 385.42 μmol), DMF (1 mL), and stirred for 2 h at 50° C. The reaction was quenched with water (2 mL), extracted with EA (2 mL×2). The combined organic layers were washed with water (2 mL) and brine (2 mL) successively, separated, then concentrated with vacuum. The residue was purified with prep-HPLC (Mobile Phase A: water (ammonium hydroxide), Mobile Phase B: A CN; Flow rate: 70 mL/min; Gradient: 35-70-90% B (2-30-60 min); 270 nm; RT: 32.698-34.423 min) to afford 4-(4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)morpholin-3-one (192) (0.051 g, 84.35 μmol, 69.89% yield) as a white solid. LCMS: m/z=605 [M+1]⁺.

¹H NMR (400 MHz, DMSO-d₆) δ 7.31-7.14 (m, 2H), 6.93-6.84 (m, 1H), 6.82-6.67 (m, 3H), 5.67-5.55 (m, 1H), 5.14 (t, J=8.7 Hz, 1H), 4.89-4.67 (m, 1H), 4.37-4.24 (m, 2H), 4.15 (d, J=8.8 Hz, 2H), 4.00-3.91 (m, 2H), 3.85-3.72 (m, 5H), 3.68-3.61 (m, 2H), 3.01 (d, J=10.8 Hz, 1H), 2.78 (s, 1H), 2.31-1.83 (m, 7H), 1.77-1.62 (m, 1H).

### Example 193

### (8-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-2,3-dihydrobenzo[b][1,4]dioxin-5-yl)dimethylphosphine oxide (193)

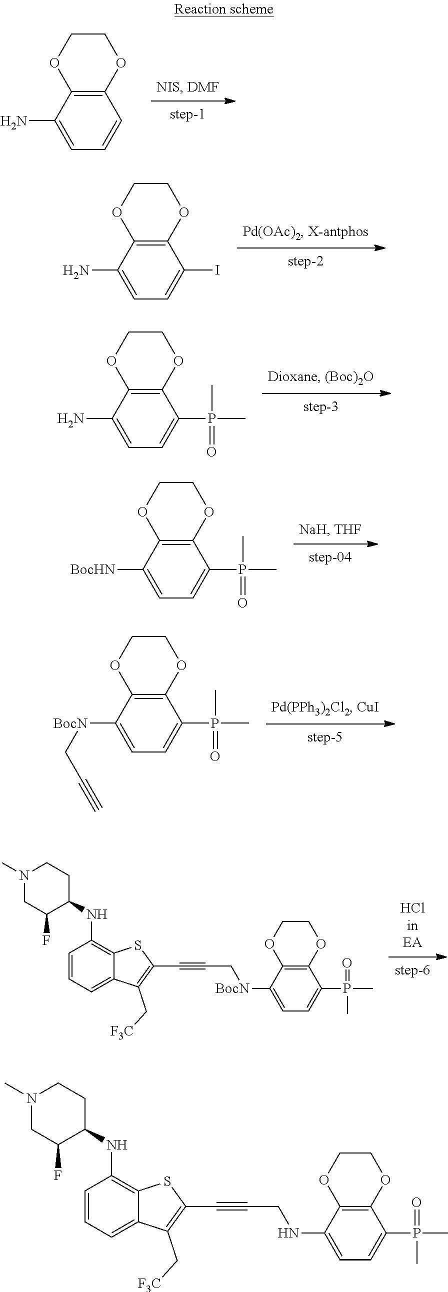

### Experimental Details

### Step 1. 8-iodo-2,3-dihydrobenzo[b][1,4]dioxin-5-amine

Into a 100-mL three necked bottle purged and maintained with an inert atmosphere of nitrogen, was placed 2,3-dihydrobenzo[b][1,4]dioxin-5-amine (2.004 g, 13.26 mmol), N,N-dimethylformamide (20 mL). The reaction mixture was cooled to −40° C. Then N-iodosuccinimide (3.132 g, 13.92 mmol) was added and stirred at the same temperature for 15 h. LCMS showed that the reaction was completed. The resulting solution was quenched with sodium bisulfite aqueous solution (10 mL) and extracted with EA (2×50 mL). The organic layer was combined and washed with brine (50 mL), dried over anhydrous Na₂SO₄, the residue was concentrated under vacuum. The residues were purified by C₁₈ chromatography column eluted with ACN/H₂O (0.05% TFA)(v/v=2/5). This resulted in 3.35 g (91.21% yield) of 8-iodo-2,3-dihydrobenzo[b][1,4]dioxin-5-amine as brown solid. LCMS: m/z=278 [M+1]⁺.

### Step 2. (8-amino-2,3-dihydrobenzo[b][1,4]dioxin-5-yl)dimethylphosphine oxide

Into a 100-mL three necked bottle purged and maintained with an inert atmosphere of nitrogen, was placed 8-iodo-2,3-dihydrobenzo[b][1,4]dioxin-5-amine (3.198 g, 11.5427 mmol), palladium (II) acetate (0.402 g, 1. 79 mmol), dimethylbisdiphenylphosphinoxanthene, (1.118 g, 1.93 mmol), N,N-diisopropylethylamine (3.184 g, 24.64 mmol), N,N-dimethylformamide (20 mL). The reaction mixture was heated to 80° C. Then methylphosphinoylmethane (4.01 g, 51.38 mmol) was added and stirred at the same temperature for 1.0 h. LCMS showed that the reaction was completed. The residue was filtered and purified by C₁₈ chromatography column eluted with ACN/H₂O (0.05% NH₄HCO₃) (v/v=1/10). This resulted in 2.22 g (84.65% yield) of (8-amino-2,3-dihydrobenzo[b][1,4]dioxin-5-yl)dimethylphosphine oxide as yellow solid. LCMS: m/z=228 [M+1]⁺

### Step 3. tert-butyl (8-(dimethylphosphoryl)-2,3-dihydrobenzo[b][1,4]dioxin-5-yl)carbamate

Into a 100-mL three necked bottle purged and maintained with an inert atmosphere of nitrogen, was placed N-(4-pyridyl)dimethylamine (0.39 g, 3.1924 mmol), (8-amino-2,3-dihydrobenzo[b][1,4]dioxin-5-yl)dimethyl phosphine oxide (2.21 g, 9.73 mmol), di-tert-butyl dicarbonate (8.84 g, 40.50 mmol), 1,4-dioxane (20 mL). The reaction mixture was stirred at 110° C. for 17 h. LCMS showed that the reaction was completed. The resulted solution was concentrated and purified by C₁₈ chromatography column eluted with ACN/H₂O (v/v=3/5). This resulted in 1.96 g (61.57% yield) of tert-butyl (8-(dimethylphosphoryl)-2,3-dihydrobenzo[b][1,4]dioxin-5-yl)carbamate as yellow solid. LCMS: m/z=328 [M+1]⁺.

### Step 4. tert-butyl (8-(dimethylphosphoryl)-2,3-dihydrobenzo[b][1,4]dioxin-5-yl)(prop-2-yn-1-yl)carbamate

Into a 25-mL three necked bottle purged and maintained with an inert atmosphere of nitrogen, was placed tert-butyl (8-(dimethylphosphoryl)-2,3-dihydrobenzo[b][1,4]dioxin-5-yl)carbamate (0.501 g, 1.53 mmol), tetra hydrofuran (10 mL). The reaction mixture was stirred at 0° C. Then sodium hydride (0.362 g, 15.09 mmol) was added. The mixture was stirred at 20° C. for 1.0 h. Then 3-bromopropyne (1.952 g, 16.41 mmol) was added to the reaction at −10° C. The reaction mixture was stirred at 70° C. for 1.5 h. The resulting solution was added to water (10 mL). The resulting solution was extracted with EA (2×50 mL). The organic layer was combined and washed with brine (50 mL), dried over anhydrous Na₂SO₄, the residue was concentrated under vacuum. The residue was purified by C₁₈ chromatography column eluted with ACN/H₂O (v/v=2/5). This resulted in 0.47 g (84.04% yield) of tert-butyl (8-(dimethylphosphoryl)-2,3-dihydrobenzo[b][1,4]dioxin-5-yl)(prop-2-yn-1-yl)carbamate as yellow oil. LCMS: m/z=366 [M+1]⁺.

### Step 5. tert-butyl (8-(dimethylphosphoryl)-2,3-dihydrobenzo[b][1,4]dioxin-5-yl)(3-(7-(((3S,4R)-3-fluoro-1-m ethylpiperidin-4-yl)amino)-3-(2,2,2-trifluoroethyl)benzo[b]thiophen-2-yl)prop-2-yn-1-yl)carbamate

Into a 8-mL three necked bottle purged and maintained with an inert atmosphere of nitrogen, was placed [1,1′-bis(diphenylphosphino)ferrocene]dichloropalladium(II) (0.046 g, 65.16 μmol), cuprous iodide (0.008 g, 42.01 μmol), triethylamine (0.049 g, 484.24 μmol), (3S,4R)-3-fluoro-N-(2-iodo-3-(2,2,2-trifluoroethyl)benzo[b]thiophen-7-yl)-1-methylpiperidin-4-amine (0.141 g, 385.92 μmol), methyl sulfoxide (2 mL). The reaction mixture was stirred at 80 refor 10 min. Then tert-butyl (8-(dimethylphosphoryl)-2,3-dihydrobenzo[b][1,4]dioxin-5-yl)(prop-2-yn-1-yl)carbamate (0.141 g, 385.92 μmol) was added and stirred at the same temperature for 1 h. LCMS showed that the reaction was completed. The resulting solution was quenched with water (200 mL) and extracted with EA (2×100 mL). The organic layer was combined and washed with brine (100 mL), dried over anhydrous Na₂SO₄, the residue was concentrated under vacuum. The residue was purified by C₁₈ chromatography column eluted with ACN/H₂O (0.05% TFA) (v/v=2/5). This resulted in 0.10 g (36.51% yield) of tert-butyl (8-(dimethylphosphoryl)-2,3-dihydrobenzo[b][1,4]dioxin-5-yl)(3-(7-(((3S,4R)-3-fluoro-1-methylpiperidin-4-yl)amino)-3-(2,2,2-trifluoroethyl)benzo[b]thiophen-2-yl)prop-2-yn-1-yl)carbamate as brown solid. LCMS: m/z=709 [M+1]⁺.

### Step 6. (8-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-2,3-dihydrobenzo[b][1,4]dioxin-5-yl)dimethylphosphine oxide (193)

Into a 4-mL round-bottom flask purged and maintained with an inert atmosphere of nitrogen, was placed tert-butyl (8-(dimethylphosphoryl)-2,3-dihydrobenzo[b][1,4]dioxin-5-yl)(3-(7-(((3S,4R)-3-fluoro-1-methylpiperidin-4-yl)amino)-3-(2,2,2-trifluoroethyl)benzo[b]thiophen-2-yl)prop-2-yn-1-yl)carbamate (0.099 g, 139.49 μmol), et hyl acetate (0.5 mL) and HCl in ethyl acetate solution (0.5 mL, 4 M). The reaction mixture was stirred at room temperature for 0.5 h. LCMS showed that the reaction was completed. Then saturated sodium hydro gen carbonate aqueous solution was added to the mixture until pH=7˜8. The resulting solution was extracted with EA (2×40 mL). The organic layer was combined, washed with brine (40 mL), dried over anhydrous Na₂SO₄ and concentrated under vacuum. The mixture was purified by preparative HPLC (Mobile Phase A: water (10 mmoL/L ammonium hydroxide), Mobile Phase B: ACN; Flow rate: 70 mL/min; Gradient: 35-71-75% B (2-30-34 min; 262 nm; RT: 29.90-31.70 min). This resulted in 0.024 g (23.24% yield) of (8-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-2,3-dihydrobenzo[b][1,4]dioxin-5-yl)dimethylphosphine oxide (193) as white solid. LCMS: m/z=6 [M+1]⁺.

¹H NMR (400 MHz, DMSO) δ 7.27 (s, 1H), 7.22 (s, 1H), 7.16-7.11 (m, 1H), 6.79-6.76 (m, 1H), 6.53-6.50 (m, 1H), 6.02-5.99 (m, 1H), 5.17-5.13 (m, 1H), 4.88-4.83 (m, 1H), 4.75-4.72 (m, 1H), 4.32 (s, 1H), 4.30 (s, 3H), 3.79 (s, 2H), 3.06-3.00 (m, 3H), 2.82-2.77 (m, 2H), 2.33 (s, 1H), 2.18 (s, 3H), 1.58 (s, 3H), 1.55 (s, 3H), 1.24 (s, 2H).

### Example 194

### (2-fluoro-4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)dimethylphosphine oxide (194)

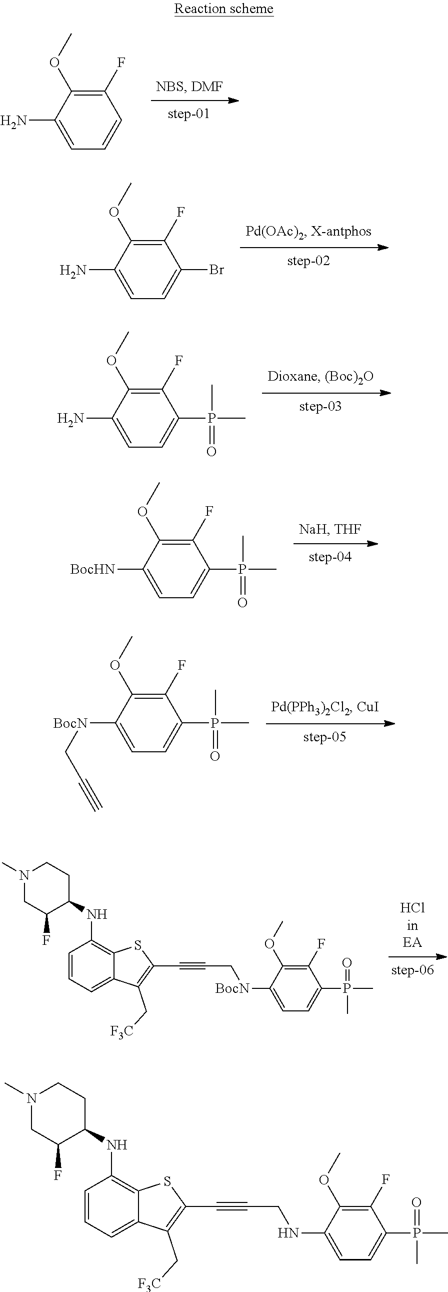

### Experimental Details

### Step 1. 4-bromo-3-fluoro-2-methoxyaniline

Into a 100-mL three necked bottle purged and maintained with an inert atmosphere of nitrogen, was placed 3-fluoro-2-methoxyaniline (1.078 g, 7.64 mmol), N,N-dimethylformamide (10 mL). The reaction mixture was cooled to −40° C. Then N-bromosuccinimide (1.472 g, 8.27 mmol) was added and stirred at the same temperature for 1 h. LCMS showed that the reaction was completed. The resulting solution was quenched with sodium bisulfite aqueous solution (10 mL) and extracted with EA (2×15 mL). The organic layer w as combined and washed with brine (20 mL), dried over anhydrous Na₂SO₄, the residue was concentrated under vacuum. The residues were purified by C₁₈ chromatography column eluted with ACN/H₂O (0.05% T FA)(v/v=2/5). This resulted in 1.31 g (77.95% yield) of 4-bromo-3-fluoro-2-methoxyaniline as brown solid. LCMS: m/z=221 [M+1]⁺.

### Step 2. (4-amino-2-fluoro-3-methoxyphenyl)dimethylphosphine oxide

Into a 50-mL three necked bottle purged and maintained with an inert atmosphere of nitrogen, was placed 4-bromo-3-fluoro-2-methoxyaniline (0.515 g, 2.34 mmol), palladium (II) acetate (0.110 g, 489.96 μmol), dimethylbisdiphenylphosphinoxanthene (0.251 g, 433.79 μmol), N,N-diisopropylethylamine (0.709 g, 5.49 mmol), N,N-dimethylformamide (10 mL). The reaction mixture was heated to 130° C. Then methylphosphinoylmethane (0.750 g, 9.61 mmol) was added and stirred at the same temperature for 1.0 h. LCMS showed that the reaction was completed. The residue was filtered and purified by C₁₈ chromatography column eluted with ACN/H₂O (0.05% NH₄HCO₃) (v/v=1/5). This resulted in 0.32 g (62.96% yield) of (4-amino-2-fluoro-3-methoxyphenyl)dimethylphosphine oxide as white solid. LCMS: m/z=218 [M+1]⁺.

Step 3. tert-butyl (4-(dimethylphosphoryl)-3-fluoro-2-methoxyphenyl)carbamate Into a 8-mL three necked bottle purged and maintained with an inert atmosphere of nitrogen, was placed (4-amino-2-fluoro-3-methoxyphenyl)dimethylphosphine oxide (0.295 g, 1.36 mmol), di-tert-butyl dicarbonate (1.372 g, 6.29 mmol), 1,4-dioxane (4 mL). The reaction mixture was stirred at 110° C. for 24 h. LCMS showed that the reaction was completed. The resulting solution was concentrated and purified by C₁₈ chromatography column eluted with ACN/H₂O (v/v=1/3). This resulted in 0.47 g (109.05% yield) of tert-butyl (4-(dimethylphosphoryl)-3-fluoro-2-methoxyphenyl)carbamate as yellow oil. LCMS: m/z=318 [M+1]⁺.

Step 4. tert-butyl (4-(dimethylphosphoryl)-3-fluoro-2-methoxyphenyl)(prop-2-yn-1-yl)carbamate Into a 25-mL three necked bottle purged and maintained with an inert atmosphere of nitrogen, was placed tert-butyl (4-(dimethylphosphoryl)-3-fluoro-2-methoxyphenyl)carbamate (0.468 g, 1.48 mmol), tetrahydrofuran (5 mL). The reaction mixture was stirred at 0° C. Then sodium hydride (0.301 g, 12.54 mmol) was added. The mixture was stirred at 20° C. for 1.0 h. Then 3-bromopropyne (1.770 g, 14.88 mmol) was added to the reaction at −10° C. The reaction mixture was stirred at 80° C. for 1.0 h. The resulting solution was added to water (5 mL). The resulting solution was extracted with EA (2×10 mL). The organic layer was combined and washed with brine (10 mL), dried over anhydrous Na₂SO₄, the residue was concentrated under vacuum. The residue was purified by C₁₈ chromatography column eluted with ACN/H₂O (v/v=1/1). This resulted in 0.279 g (53.23% yield) of tert-butyl (4-(dimethylphosphoryl)-3-fluoro-2-methoxyphenyl)(prop-2-yn-1-yl)carba mate as yellow oil. LCMS: m/z=356 [M+1]⁺.

### Step 5. tert-butyl (4-(dimethylphosphoryl)-3-fluoro-2-methoxyphenyl)(3-(7-(((3S,4R)-3-fluoro-1-methylpiperidin-4-yl)amino)-3-(2,2,2-trifluoroethyl)benzo[b]thiophen-2-yl)prop-2-yn-1-yl)carbamate

Into a 8-mL three necked bottle purged and maintained with an inert atmosphere of nitrogen, was placed triethylamine (0.298 g, 2.95 mmol), tert-butyl (4-(dimethylphosphoryl)-3-fluoro-2-methoxyphenyl)(prop-2-yn-1-yl)carbamate(0.122 g, 343.33 μmol), cuprous iodide (0.006 g, 31.50 μmol), (3S,4R)-3-fluoro-N-(2-iodo-3-(2,2,2-trifluoroethyl)benzo[b]thiophen-7-yl)-1-methylpiperidin-4-amine (0.119 g, 251.97 μmol), [1,1′-bis(diphenylphosphino)ferrocene]dichloropalladium(II) (0.022 g, 31.16 μmol), methyl sulfoxide (2 mL). The reaction mixture was stirred at 80 refor 1 h. LCMS showed that the reaction was completed. The resulting solution was quenched with water (20 mL) and extracted with EA (2×10 mL). The organic layer was combined and washed with brine (10 mL), dried over anhydrous Na₂SO₄, the residue was concentrated under vacuum. The residue was purified by C₁₈ chromatography column eluted with ACN/H₂O (0.05% TFA) (v/v=2/5). This resulted in 0.185 g (104.93% yield) of tert-butyl (4-(dimethylphosphoryl)-3-fluoro-2-methoxyphenyl)(3-(7-(((3S,4R)-3-fluoro-1-methylpiperidin-4-yl)amino)-3-(2,2,2-trifluoroethyl)benzo[b]thiophen-2-yl)prop-2-yn-1-yl)carbamate as brown oil. LCMS: m/z=700 [M+1]⁺.

### Step 6. (2-fluoro-4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)dimethylphosphine oxide (194)

Into a 2-mL round-bottom flask purged and maintained with an inert atmosphere of nitrogen, was placed tert-butyl (4-(dimethylphosphoryl)-3-fluoro-2-methoxyphenyl)(3-(7-(((3S,4R)-3-fluoro-1-methylpiperidin-4-yl)amino)-3-(2,2,2-trifluoroethyl)benzo[b]thiophen-2-yl)prop-2-yn-1-yl)carbamate (0.183 g, 261.54 μmol), ethyl acetate (5 mL) and HCl in ethyl acetate solution (5 mL, 4 M). The reaction mixture was stirred at room temperature for 0.5 h. LCMS showed that the reaction was completed. Then saturated sodium hydrogen carbonate aqueous solution was added to the mixture until pH=7˜8. The resulting solution was extracted with eth yl acetate (2×10 mL). The organic layer was combined, washed with brine (10 mL), dried over anhydrous Na₂SO₄ and concentrated under vacuum. The mixture was purified by preparative HPLC (Mobile Phase A: water (10 mmoL/L ammonium hydroxide), Mobile Phase B: ACN; Flow rate: 70 mL/min; Gradient: 35-75-75% B (2-30-32 min; 260 nm; RT: 27.98-30.10 min). This resulted in 0.074 g (47.19% yield) of (2-fluoro-4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)dimethylphosphine oxide (194) as white solid. LCMS: m/z=600 [M+1]⁺.

¹H NMR (400 MHz, DMSO) δ 7.32-7.24 (m, 2H), 7.20 (d, J=7.9 Hz, 1H), 6.78 (d, J=7.8 Hz, 2H), 6.61 (t, J=6.3 Hz, 1H), 5.15 (d, J=8.3 Hz, 1H), 4.87-4.71 (m, 1H), 4.36 (d, J=6.2 Hz, 2H), 3.82 (d, J=10.6 Hz, 1H), 3.79-3.74 (m, 4H), 3.72-3.58 (m, 1H), 3.03 (t, J=10.9 Hz, 1H), 2.79 (d, J=10.4 Hz, 1H), 2.32-2.24 (m, 1H), 2.18 (s, 3H), 2.08 (t, J=11.6 Hz, 1H), 2.00-1.91 (m, 1H), 1.72 (d, J=10.7 Hz, 1H), 1.64 (s, 3H), 1.61 (s, 3H).

### Example 195

### (4-((3-(7-((3-oxaspiro[5.5]undecan-9-yl)amino)-3-(2,2,2-trifluoroethyl)benzo[b]thiophen-2-yl)-prop-2-yn-1-yl)amino)-3-methoxyphenyl)dimethylphosphine oxide (195)

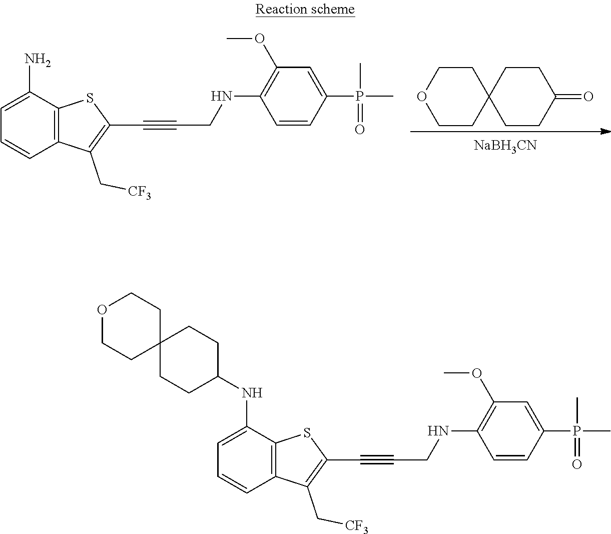

### Experiment Details

### Step 1. (4-((3-(7-((3-oxaspiro[5.5]undecan-9-yl)amino)-3-(2,2,2-trifluoroethyl)benzo[b]thiophen-2-yl)prop-2-yn-1-yl)amino)-3-methoxyphenyl)dimethylphosphine oxide (195)

Into an opened 4 mL flask was placed (4-((3-(7-amino-3-(2,2,2-trifluoroethyl)benzo[b]thiophen-2-yl)prop-2-yn-1-yl)amino)-3-methoxyphenyl)dimethylphosphine oxide (0.048 g, 102.90 μmol), 4-(3-methoxy-4-(prop-2-yn-1-ylamino)phenyl)morpholin-3-one (0.039 g, 231.82 μmol), and stirred for 2 h at 120i. The reaction was cooled to room temperature. NaBH₃CN (4.4121 mg, 102.90 μmol), EtOH (1 mL), acetic acid (0.01 mL) was added to the reaction, and stirred for additional 0.5 h at 50° C. The reaction was quenched with water (2 mL), extracted with EA (2 mL×2). The combined organic layer was washed with water (2 mL) and brine (2 mL) successively, separated, then concentrated under vacuum. The residue was purified with prep-HPLC (Mobile Phase A: water (ammonium hydroxide), Mobile Phase B: ACN; Flow rate: 70 mL/min; Gradient: 40-80-90% B (2-30-38 min); 264 nm; RT: 35.49-36.97 min) to afford (4-((3-(7-((3-oxaspiro[5.5]undecan-9-yl)amino)-3-(2,2,2-trifluoroethyl)benzo[b]thiophen-2-yl)prop-2-yn-1-yl)amino)-3-methoxyphenyl)dimethylphosphine oxide (195) (0.027 g, 43.64 μmol, 42.41% yield) as white solid. LCMS: m/z=619 [M+1]⁺.

¹H NMR (400 MHz, DMSO-d₆) δ 7.30-7.09 (m, 4H), 6.89-6.80 (m, 1H), 6.65 (d, J=7.8 Hz, 1H), 6.06 (t, J=6.4 Hz, 1H), 5.24 (d, J=8.3 Hz, 1H), 4.33 (d, J=6.4 Hz, 2H), 3.80 (d, J=36.0 Hz, 5H), 3.54 (t, J=5.3 Hz, 4H), 1.76 (d, J=11.5 Hz, 4H), 1.58 (d, J=13.1 Hz, 6H), 1.54-1.41 (m, 4H), 1.36-1.15 (m, 5H).

### Example 196&197 (3-methoxy-4-((3-(7-(((1S,5R)-7-methyl-3-oxa-7-azabicyclo[3.3.1]nonan-9-yl)amino)-3-(2,2,2-trifluoroethyl)benzo[b]thiophen-2-yl)prop-2-yn-1-yl)amino)phenyl)dimethylphosphine oxide (196) and (3-methoxy-4-((3-(7-(((1R,5S)-7-methyl-3-oxa-7-azabicyclo[3.3.1]nonan-9-yl)amino)-3-(2,2,2-trifluoroethyl)benzo[b]thiophen-2-yl) prop-2-yn-1-yl)amino)phenyl)dimethylphosphine oxide (197)

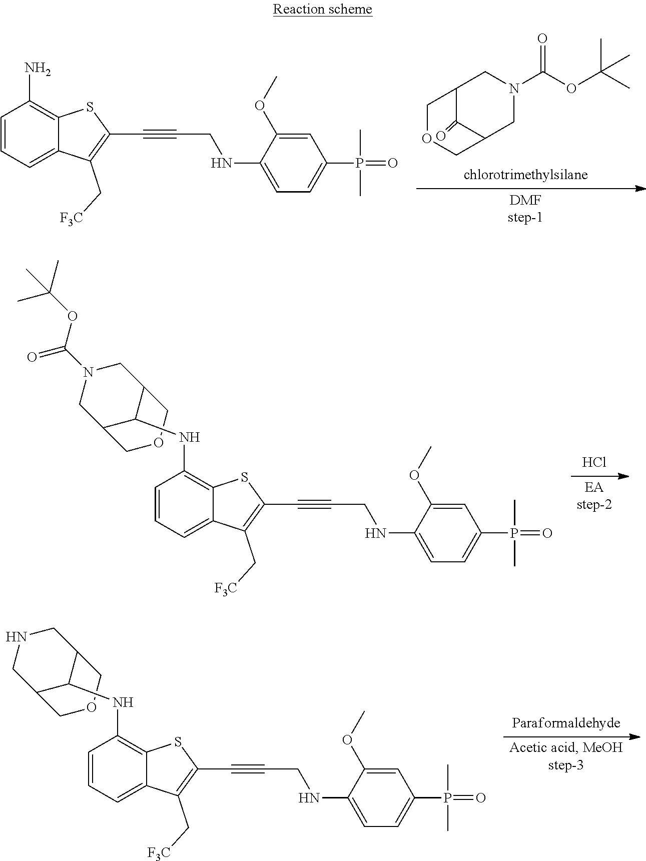

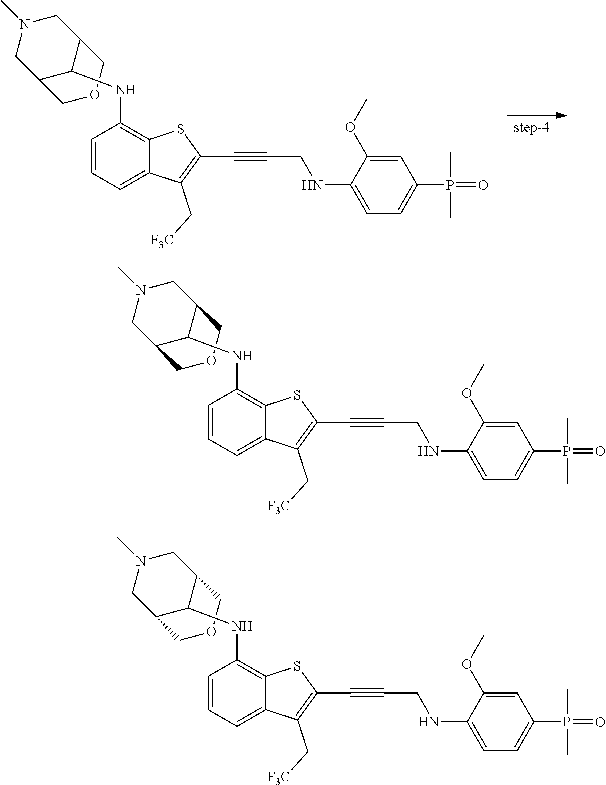

### Step 1. tert-butyl 9-((2-(3-((4-(dimethylphosphoryl)-2-methoxyphenyl)amino)prop-1-yn-1-yl)-3-(2,2,2-trifluoroethyl)benzo[b]thiophen-7-yl)amino)-3-oxa-7-azabicyclo[3.3.1]nonane-7-carboxylate

Into a 25-mL three necked bottle and maintained with an inert atmosphere of nitrogen, (4-((3-(7-amino-3-(2,2,2-trifluoroethyl)benzo[b]thiophen-2-yl)prop-2-yn-1-yl)amino)-3-methoxyphenyl)dimethylphosphine oxide (0.213 g, 456.63 umol), tert-butyl 9-oxo-3-oxa-7-azabicyclo[3.3.1]nonane-7-carboxylate (0.246 g, 1.01 mmol), chlorotrimethylsilane (0.711 g, 6.54 mmol), DMF (2 mL). The reaction was stirred under nitrogen atomosphere at 0° C. for 1 h. Borane-tetrahydrofuran complex (1.5 ml) was added. The reaction was stirred under nitrogen atmosphere at 0° C. for 1 h. The residue was dissolved in MeOH (2 mL). The reaction mixture was purified by C18 column eluted with ACN/H₂O (v/v=3/2), filtered and concentrated under vacuum. This resulted in 0.277 g, (87.69% yield) of tert-butyl 9-((2-(3-((4-(dimethylphosphoryl)-2-methoxyphenyl)amino)prop-1-yn-1-yl)-3-(2,2,2-trifluoroethyl)benzo[b]thiophen-7-yl)amino)-3-oxa-7-azabicyclo[3.3.1]nonane-7-carboxylate as yellow solid. LCMS: m/z=692 [M+1]⁺.

### Step 2. (4-((3-(7-((3-oxa-7-azabicyclo[3.3.1]nonan-9-yl)amino)-3-(2,2,2-trifluoroethyl)benzo[b]thiophen-2-yl) prop-2-yn-1-yl)amino)-3-methoxyphenyl)dimethylphosphine oxide

Into a 25-mL round-bottom flask purged and maintained with an inert atmosphere of nitrogen, was placed tert-butyl 9-((2-(3-((4-(dimethylphosphoryl)-2-methoxyphenyl)amino)prop-1-yn-1-yl)-3-(2,2,2-trifluoroethyl)benzo[b]thiophen-7-yl)amino)-3-oxa-7-azabicyclo[3.3.1]nonane-7-carboxylate (0.262 g, 378.75 umol), ethyl acetate (3 mL) and HCl in EA solution (3 mL, 4 M). The reaction mixture was stirred at room temperature for 1.5 h. The residue was dissolved in saturated NaHCO₃ solution (20 mL). The resulting solution was extracted with EA (3×30 mL) and the organic layers were combined, washed with brine (2×20 mL), dried over anhydrous sodium sulfate, filtered and concentrated under reduced pressure to afford desired product. This resulted in 0.220 g (108.36% yield) of (4-((3-(7-((3-oxa-7-azabicyclo[3.3.1]nonan-9-yl)amino)-3-(2,2,2-trifluoroethyl)benzo[b]thiophen-2-yl)prop-2-yn-1-yl)amino)-3-methoxyphenyl)dimethylphosphine oxide as yellow oil. LCMS: m/z=592 [M+1]⁺.

### Step 3. (3-methoxy-4-((3-(7-((7-methyl-3-oxa-7-azabicyclo[3.3.1]nonan-9-yl)amino)-3-(2,2,2-trifluoroethyl)benzo[b]thiophen-2-yl)prop-2-yn-1-yl)amino)phenyl)dimethylphosphine oxide

Into a 25-mL three necked bottle purged and maintained with an inert atmosphere of nitrogen, was placed (4-((3-(7-((3-oxa-7-azabicyclo[3.3.1]nonan-9-yl)amino)-3-(2,2,2-trifluoroethyl)benzo[b]thiophen-2-yl)prop-2-yn-1-yl)amino)-3-methoxyphenyl)dimethylphosphine oxide (0.152 g, 256.92 umol), Paraformaldehyde (0.050 g, 0.66 mmol), Acetic acid (0.2 mL), MeOH (2 mL) was added. The mixture was stirred at rt for 2 h. Sodium cyanoboronhydride, (0.092 g, 2.14 mmol) was added. The reaction mixture was stirred at room temperature for 1 h. The reaction mixture was filtered through celite pad and celite pad was washed with ACN (3×20 mL). The filterate was then concentrated under reduced pressure to afford the crude product. The crude product was purified by preparative HPLC (Mobile Phase A: water (0.1% ammonium hydroxide), Mobile Phase B: ACN; Flow rate: 40 mL/min; Gradient: 30-50-50% B (2-30-34 min); 262 nm; RT: 25.38-34.00) to provide the desired product. This resulted in 0.096 g (69.69% yield) of (3-methoxy-4-((3-(7-((7-methyl-3-oxa-7-aza bicyclo[3.3.1]nonan-9-yl)amino)-3-(2,2,2-trifluoroethyl)benzo[b]thiophen-2-yl)prop-2-yn-1-yl)amino)phenyl)dimethylphosphine oxide as yellow solid. LCMS: m/z=606 [M+1]⁺.

### Step 5. (3-methoxy-4-((3-(7-(((1S,5R)-7-methyl-3-oxa-7-azabicyclo[3.3.1]nonan-9-yl)amino)-3-(2,2,2-trifluoro ethyl)benzo[b]thiophen-2-yl)prop-2-yn-1-yl)amino)phenyl)dimethylphosphine oxide (196) and (3-methoxy-4-((3-(7-(((1R,5S)-7-methyl-3-oxa-7-azabicyclo[3.3.1]nonan-9-yl)amino)-3-(2,2,2-trifluoroethyl)benzo[b]thiophen-2-yl)prop-2-yn-1-yl)amino)phenyl)dimethylphosphine oxide (197)

The sample (3-methoxy-4-((3-(7-((7-methyl-3-oxa-7-azabicyclo[3.3.1]nonan-9-yl)amino)-3-(2,2,2-trifluoroethyl)benzo[b]thiophen-2-yl)prop-2-yn-1-yl)amino)phenyl)dimethylphosphine oxide (0.085 g, 140.31 μmol) was separated by prep-chiral (Column:IG, Mobile Phase A: n-Hexane, Mobile Phase B: EtOH; Flow rate: 20 mL/min; (HeX:DCM=3:1):EtOH (V/V=50:50); 220 nm RT: 6.626 min) to provide 0.047 g (55.29% yield) (3-methoxy-4-((3-7-(((1S,5R)-7-methyl-3-oxa-7-azabicyclo[3.3.3.]nonan-9-yl)amino)-3-2,2,2-trifluoroethyl)benzo[b]thiophen-2-yl)prop-2-yn-1-yl)amino)phenyl)dimethylphosphine oxide (196) as white solid. LCMS: m/z=606 [M+1]⁺.

¹H NMR (400 MHz, DMSO-d₆) δ 7.33-7.19 (m, 3H), 7.14-7.11 (m, 1H), 6.85-6.81 (m, 1H), 6.74 (d, J=7.6 Hz, 1H), 6.08 (t, J=6.3 Hz, 1H), 5.29 (d, J=5.2 Hz, 1H), 4.34 (d, J=6.3 Hz, 2H), 3.91-3.69 (m, 10H), 2.68-2.53 (m, 4H), 2.10 (s, 3H), 1.97 (s, 2H), 1.58 (d, J=13.2 Hz, 6H).

The sample (3-methoxy-4-((3-(7-((7-methyl-3-oxa-7-azabicyclo[3.3.1]nonan-9-yl)amino)-3-(2,2,2-trifluoroethyl)benzo[b]thiophen-2-yl)prop-2-yn-1-yl)amino)phenyl)dimethylphosphine oxide (0.085 g, 140.31 μmol) was separated by prep-chiral (Column:IG, Mobile Phase A: n-Hexane, Mobile Phase B: EtOH; Flow rate: 20 mL/min; (HeX:DCM=3:1):EtOH (V/V=50:50); 220 nm; RT: 7.871 min) to provide 0.046 g (54.11% yield) (3-methoxy-4-((3-(7-(((1R,5S)-7-methyl-3-oxa-7-azabicyclo[3.3.1]nonan-9-yl)amino)-3-(2,2,2-trifluoroethyl)benzo[b]thiophen-2-yl)prop-2-yn-1-yl)amino)phenyl)dimethylphosphine oxide (197) as white solid. LCMS: m/z=606 [M+1]⁺.

¹H NMR (400 MHz, DMSO-d₆) δ 7.25-7.22 (m, 3H), 7.16-7.13 (m, 1H), 6.86-6.82 (m, 1H), 6.64 (d, J=7.6 Hz, 1H), 6.08 (t, J=6.4 Hz, 1H), 5.41 (d, J=3.8 Hz, 1H), 4.34 (d, J=6.4 Hz, 2H), 3.98 (d, J=11.3 Hz, 2H), 3.82 (d, J=25.1 Hz, 5H), 3.54-3.51 (m, 3H), 2.98 (d, J=10.8 Hz, 2H), 2.37 (d, J=10.9 Hz, 2H), 2.14 (s, 3H), 1.86 (s, 2H), 1.58 (d, J=13.1 Hz, 6H).

### Example 198

### (7-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)benzofuran-4-yl)dimethylphosphine oxide (198)

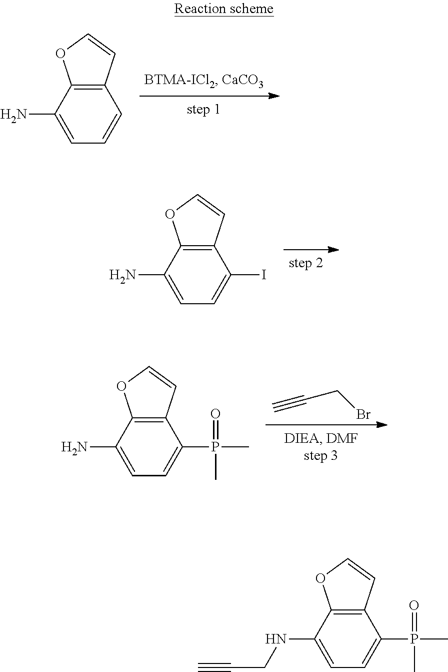

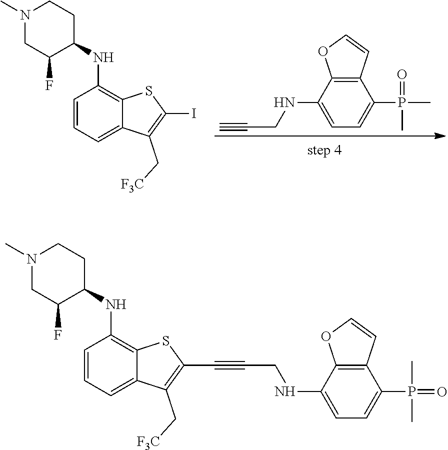

### Experimental Details

### Step 1. 4-iodobenzofuran-7-amine

Into a 100-mL three necked bottle purged and maintained with an inert atmosphere of nitrogen, was placed benzofuran-7-amine (1.03 g, 7.72 mmol), DCM (5 mL), CaCO₃ (1.50 g, 14.69 mmol), MeOH (5 mL). The reaction mixture was stirred at 50° C. Then Benzyltrimethylammonium dichloroiodate (3.25 g, 135.43 mm ol) was added. The mixture was stirred at 50° C. for 12 h. The resulting solution was added to water (100 mL). The resulting solution was extracted with EA (2×150 mL). The organic layers combined and washed with brine (50 mL), dried over anhydrous Na₂SO₄, the residue was concentrated under vacuum. The residue was applied onto a C₁₈ column eluted with ACN/H₂O (0.01% TFA) (v:v=1:1), Flow rate: 80 mL/min. This resulted in 804.00 mg (40.16% yield) of 4-iodobenzofuran-7-amine as yellow solid. LCMS: m/z=260 [M+1]⁺

### Step 2. (7-aminobenzofuran-4-yl)dimethylphosphine oxide

Into a 40-mL reaction vial purged and maintained with an inert atmosphere of nitrogen, was placed 4-iodobenzofuran-7-amine (738.00 mg, 2.84 mmol), Pd(OAc)₂ (84.00 mg, 0.37 mmol), Xantphos (313.00 mg, 0.54 mmol), DIEA (706.00 mg, 5.46 mmol) and DMF (10 mL) stirred at 110° C. for 5 min, then added dimethylphosphine oxide (528.00 mg, 6.76 mmol) in DMF (2 mL). The reaction mixture was stirred at 110° C. for 2 h. The resulting solution was concentrated under vacuum. The residue was applied onto a C₁₈ column eluted with ACN/H₂O (0.01% TFA) (v:v=1:1), Flow rate: 80 mL/min. This resulted in 500.00 mg (83.90% yield) of (7-aminobenzofuran-4-yl)dimethylphosphine oxide as yellow oil. LCMS: m/z=210 [M+1]⁺

### Step 3. dimethyl(7-(prop-2-yn-1-ylamino)benzofuran-4-yl)phosphine oxide

Into a 250-mL three necked bottle purged and maintained with an inert atmosphere of nitrogen, was placed (7-aminobenzofuran-4-yl)dimethylphosphine oxide (500.00 mg, 2.39 mmol), NMP (10 mL), 3-bromoprop-1-yne (1.12 g, 9.43 mmol), DIEA (1.23 g, 9.56 mmol). The reaction mixture was stirred at 80° C. for 4 h. The residue was concentrated under vacuum. The residue was applied onto a C₁₈ column eluted with ACN/H₂O (0.01% TFA) (v:v=1:1), Flow rate: 80 mL/min. This resulted in 300.00 mg (50.76% yield) of dimeth yl(7-(prop-2-yn-1-ylamino)benzofuran-4-yl)phosphine oxide as brown solid. LCMS: m/z=248 [M+1]⁺

### Step 4. (7-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)benzofuran-4-yl)dimethylphosphine oxide (198)

Into a 50-mL three necked bottle and maintained with an inert atmosphere of nitrogen, was placed (3S,4R)-3-fluoro-N-(2-iodo-3-(2,2,2-trifluoroethyl)benzo[b]thiophen-7-yl)-1-methylpiperidin-4-amine (114.00 mg, 0.24 mmol), Pd(PPh₃)₂Cl₂ (29.00 mg, 0.041 mmol), DIEA (120.00 mg, 0.93 mmol), CuI (42.00 mg, 0.22 mmol), DMSO (6 mL) and dimethyl(7-(prop-2-yn-1-ylamino)benzofuran-4-yl)phosphine oxide (130.00 mg, 0.52 mmol). The reaction mixture was stirred at 50° C. for 3 h. The residue was applied onto a C₁₈ column eluted with ACN/H₂O (0.01% TFA) (v:v=1:1), Flow rate: 80 m/min. the residue was concentrated under vacuum. The resulting crude product was further purified by pre-HPLC with ACN/H₂O (0.1% ammonium hydroxide), Flow rate: 70 mL/min; Gradient: 35-68-71% B (2-30-33 min); 268 nm; RT: 30.35-32.77). This resulted in 70.00 mg (49.02% yield) of (7-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)benzofuran-4-yl)dimethylphosphine oxide (198) as yellow solid. LCMS: m/z=592 [M+1]⁺

¹H NMR (400 MHz, DMSO-d₆) δ 8.06 (s, 1H), 7.50-7.40 (m, 1H), 7.31-7.23 (m, 2H), 7.22-7.17 (m, 1H), 6.95-6.86 (m, 1H), 6.83-6.75 (m, 2H), 5.13 (d, J=8.4 Hz, 1H), 4.79 (d, J=49.4 Hz, 1H), 4.46 (d, J=6.1 Hz, 2H), 3.90-3.75 (m, 2H), 3.70-3.55 (m, 1H), 3.10-2.90 (m, 1H), 2.79 (d, J=11.2 Hz, 1H), 2.30-2.15 (m, 4H), 2.12-2.03 (m, 1H), 2.00-1.86 (m, 1H), 1.76-1.70 (m, 1H), 1.66 (d, J=13.1 Hz, 6H).

### Example 199

### 5-(dimethylphosphoryl)-2-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)benzamide (199)

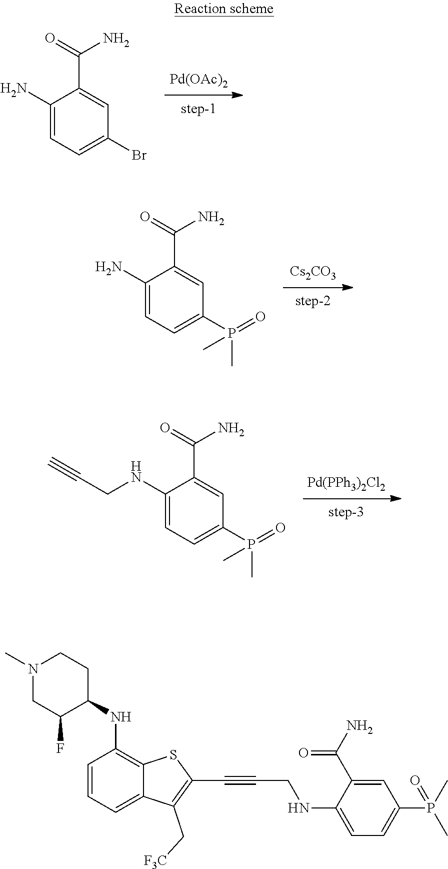

### Experimental Details

### Step 1. 2-amino-5-(dimethylphosphoryl)benzamide

Into a 100-mL three necked bottle purged and maintained with an inert atmosphere of nitrogen, was placed 2-amino-5-bromobenzamide (4.96 g, 23.06 mmol), Pd(OAc)₂ (2.70 g, 3.82 mmol), Xantphos (3.03 g, 3.72 mmol), DIEA (5.57 g, 43.10 mmol) and DMF (20 mL). The reaction mixture was stirred at 130° C. for 0.5 h. Then was added Dimethylphosphine oxide (5.57 g, 71.36 mmol). The reaction mixture was stirred at 130° C. for another 1 h. LCMS showed that the reaction was completed. The reaction mixture was purified by C18 column eluted with ACN/H₂O (v/v=2/8). This resulted in 2.21 g (49.48% yield) of 2-amino-5-(dimethylphosphoryl)benzamide as red oil. LCMS: m/z=213 [M+1]⁺.

### Step 2. 5-(dimethylphosphoryl)-2-(prop-2-yn-1-ylamino)benzamide

Into a 50-mL flask was placed 2-amino-5-(dimethylphosphoryl)benzamide (1.59 g, 7.49 mmol), 3-Bromopropyne (1.43 g, 12.04 mmol), Cs₂CO₃ (2.75 g, 8.44 mmol), KI (1.37 g, 8.25 mmol), DMF (5 mL). The reaction mixture was stirred at 80° C. for 16 h. LCMS showed that the reaction was completed. The reaction mixture was purified by C18 column eluted with ACN/H₂O (v/v=2/8). This resulted in 0.546 g (29.12% yield) of 5-(dimethylphosphoryl)-2-(prop-2-yn-1-ylamino)benzamide as red solid. LCMS: m/z=251 [M+1]⁺

### Step 3. 5-(dimethylphosphoryl)-2-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)benzamide (199)

Into a 50-mL three necked bottle and maintained with an inert atmosphere of nitrogen, was placed (3S,4R)-3-fluoro-N-(2-iodo-3-(2,2,2-trifluoroethyl)benzo[b]thiophen-7-yl)-1-methylpiperidin-4-amine (0.087 g, 0.18 m mol), 5-(dimethylphosphoryl)-2-(prop-2-yn-1-ylamino)benzamide (0.087 g, 0.35 mmol), CuI (0.040 g, 0.21 m mol), Pd(PPh₃)₂Cl₂ (0.061 g, 0.086 mmol), DIEA (0.126 g, 0.97 mmol) and DMSO (5 mL). The reaction mixture was stirred at room temperature for 1 h. LCMS showed that the reaction was completed. The mixture was purified by preparative HPLC (Mobile Phase A: water (10 mmoL/L ammonium hydroxide), Mobil e Phase B: ACN; Flow rate: 40 mL/min; Gradient: 35-66-66% B (2-30-60 min); 262 nm; RT: 23.84-25.12 min). This resulted in 0.035 g (31.95% yield) of 5-(dimethylphosphoryl)-2-((3-(7-(((3S,4R)-3-fluoro-1-methyl piperidin-4-yl)amino)-3-(2,2,2-trifluoroethyl)benzo[b]thiophen-2-yl)prop-2-yn-1-yl)amino)benzamide (199) as off white solid. LCMS: m/z=595 [M+1]⁺.

¹H NMR (400 MHz, DMSO) δ 8.74 (t, J=5.7 Hz, 1H), 8.08 (s, 1H), 7.93 (d, J=12.0 Hz, 1H), 7.68 (t, J=9.5 Hz, 1H), 7.37 (s, 1H), 7.31-7.17 (m, 2H), 7.00 (d, J=8.5 Hz, 1H), 6.79 (d, J=7.4 Hz, 1H), 5.15 (d, J=8.4 Hz, 1H), 4.79 (d, J=49.2 Hz, 1H), 4.48 (d, J=5.8 Hz, 2H), 3.84 (q, J=11.1 Hz, 2H), 3.74-3.57 (m, 1H), 3.03 (t, J=10.6 Hz, 1H), 2.79 (d, J=10.7 Hz, 1H), 2.27 (d, J=13.3 Hz, 1H), 2.18 (s, 3H), 2.08 (t, J=11.3 Hz, 1H), 2.00-1.89 (m, 1H), 1.72 (d, J=12.3 Hz, 1H), 1.62 (s, 3H), 1.59 (s, 3H).

### Example 200

### (4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-(1H-pyrazol-3-yl)phenyl)dimethylphosphine oxide (200)

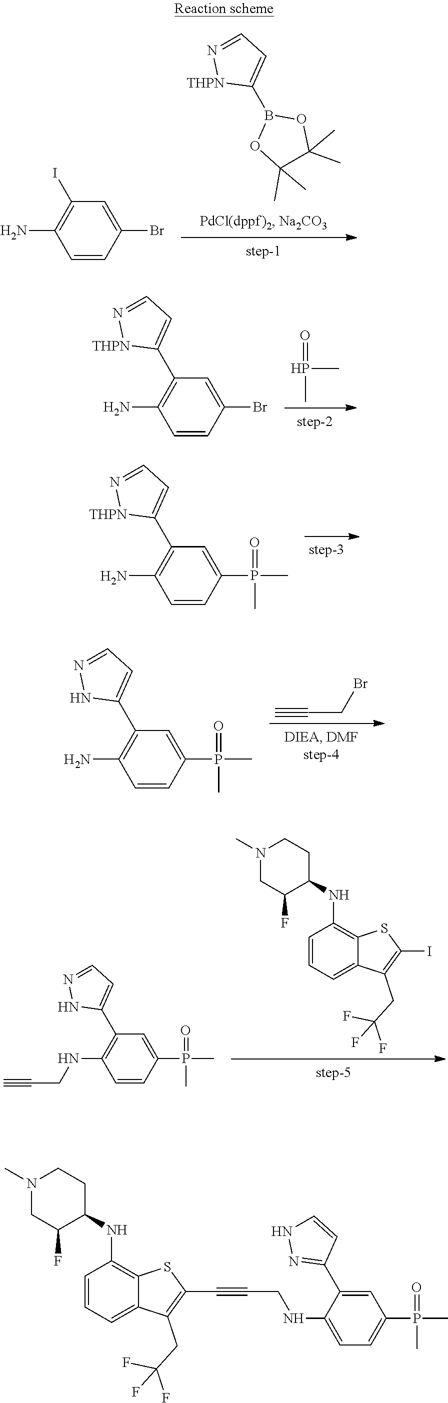

### Step 1. 4-bromo-2-(1-(tetrahydro-2H-pyran-2-yl)-1H-pyrazol-5-yl)aniline

Into a 500-mL three necked bottle purged and maintained with an inert atmosphere of nitrogen, was placed 4-bromo-2-iodoaniline (10.05 g, 33.73 mmol), 1-(tetrahydro-2H-pyran-2-yl)-5-(4,4,5,5-tetramethyl-1,3,2-dioxaborolan-2-yl)-1H-pyrazole (11.04 g, 39.69 mmol), PdCl(dppf)₂ (3.03 g, 4.14 mmol), Na₂CO₃ (15.64 g, 147. 56 mmol), 1,4-dioxane (150 mL) and H₂O (30 mL). The reaction mixture was stirred at 100° C. for 1.5 h. The resulting solution was added to water (300 mL). The resulting solution was extracted with EA (2×300 mL). The organic layers combined and washed with brine (100 mL), dried over anhydrous Na₂SO₄, the residue was concentrated under vacuum. The residue was applied onto a silica gel column eluted with EA:hexane (v:v=3:1). This resulted in 6.00 g (55.20% yield) of 4-bromo-2-(1-(tetrahydro-2H-pyran-2-yl)-1H-pyrazol-5-yl)aniline as brown oil. LCMS: m/z=322 [M+1]⁺

### Step 2. (4-amino-3-(1-(tetrahydro-2H-pyran-2-yl)-1H-pyrazol-5-yl)phenyl)dimethylphosphine oxide

Into a 250-mL three necked bottle purged and maintained with an inert atmosphere of nitrogen, was placed 4-bromo-2-(1-(tetrahydro-2H-pyran-2-yl)-1H-pyrazol-5-yl)aniline (6.11 g, 18.96 mmol), Palladium (II) acetate (1.00 mg, 4.47 mmol), Dimethylbisdiphenylphosphinoxanthene (3.27 g, 5.65 mmol), DIEA (12.32 g, 95.31 mmol), DMF (100 mL). The mixture was stirred at 110° C. for 5 min. Then dimethylphosphine oxide (10.34 g, 132.49 mmol) was added. The mixture was stirred at 110° C. for 3 h. The reaction mixture was filter ed. The residue was applied onto a C₁₈ column eluted with ACN/H₂O (0.01% TFA) (v:v=1:1), Flow rate: 100 m/min. This resulted in 3.5 g (57.78% yield) of (4-amino-3-(1-(tetrahydro-2H-pyran-2-yl)-1H-pyrazol-5-yl)phenyl)dimethylphosphine oxide as yellow solid. LCMS: m/z=320 [M+1]⁺

### Step 3. (4-amino-3-(1H-pyrazol-5-yl)phenyl)dimethylphosphine oxide

Into a 500-mL three necked bottle purged and maintained with an inert atmosphere of nitrogen, was placed (4-amino-3-(1-(tetrahydro-2H-pyran-2-yl)-1H-pyrazol-5-yl)phenyl)dimethylphosphine oxide (3.025 g, 9.47 m mol), MeOH (30 mL) and HCl (60 mL). The reaction mixture was stirred at room temperature for 12 h. The resulting solution was concentrated under vacuum. This resulted in 2.50 g (97.14% yield) of (4-amino-3-(1H-pyrazol-5-yl)phenyl)dimethylphosphine oxide as yellow solid. LCMS: m/z=236 [M+1]⁺

### Step 4. dimethyl(4-(prop-2-yn-1-ylamino)-3-(1H-pyrazol-5-yl)phenyl)phosphine oxide

Into a 250-mL three necked bottle purged and maintained with an inert atmosphere of nitrogen, was placed (4-amino-3-(1H-pyrazol-5-yl)phenyl)dimethylphosphine oxide (1.00 g, 4.26 mmol), 3-bromoprop-1-yne (1.05 g, 8.81 mmol), DIEA (3.75 g, 29.03 mmol), MeCN (20 mL). The reaction mixture was stirred at 80° C. for 4 h. The residue was concentrated under vacuum. The residue was applied onto a C₁₈ column eluted with ACN/H₂O (0.01% TFA) (v:v=1:1), Flow rate: 100 m/min. This resulted in 300.00 mg (25.74% yield) of dimethyl(4-(prop-2-yn-1-ylamino)-3-(1H-pyrazol-5-yl)phenyl)phosphine oxide as brown solid. LCMS: m/z=274 [M+1]⁺

### Step 5. (4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-(1H-pyrazol-3-yl)phenyl)dimethylphosphine oxide (200)

Into a 50-mL three necked bottle purged and maintained with an inert atmosphere of nitrogen, was placed (3S,4R)-3-fluoro-N-(2-iodo-3-(2,2,2-trifluoroethyl)benzo[b]thiophen-7-yl)-1-methylpiperidin-4-amine (228.00 mg, 0.48 mmol), dimethyl(4-(prop-2-yn-1-ylamino)-3-(1H-pyrazol-5-yl)phenyl)phosphine oxide (303.00 mg, 1.10 mmol), Pd(PPh₃)₂Cl₂ (70.00 mg, 0.099 mmol), CuI (133.00 mg, 0.70 mmol), DIEA (457.00 mg, 3.53 mmol), DMSO (8 mL). The reaction mixture was stirred at 50° C. for 3 h. The residue was applied onto a C₁₈ column eluted with ACN/H₂O (0.01% TFA) (v:v=1:1), Flow rate: 80 mL/min. the residue was concentrated under vacuum. The resulting crude product was further purified by pre-HPLC with ACN/H₂O (0.1% ammonium hydroxide), Flow rate: 70 mL/min; Gradient: 35-67-67% B (2-30-33 min); 268 nm; RT: 29.55-31.48). This resulted in 74.00 mg (24.81% yield) of (4-((3-(7-(((3S,4R)-3-fluoro-1-methylpiperidin-4-yl)amino)-3-(2,2,2-tri fluoroethyl)benzo[b]thiophen-2-yl)prop-2-yn-1-yl)amino)-3-(1H-pyrazol-3-yl)phenyl)dimethylphosphine oxide (200) as yellow solid. LCMS: m/z=618 [M+1]⁺

¹H NMR (400 MHz, DMSO-d₆) δ 13.05 (s, 1H), 8.53-8.45 (m, 1H), 7.93 (d, J=11.9 Hz, 2H), 7.89 (s, 1H), 7.59-7.50 (m, 1H), 7.30-7.20 (m, 2H), 7.07-7.02 (m, 1H), 6.87 (s, 1H), 6.78 (d, J=7.6 Hz, 1H), 5.15 (d, J=8.5 Hz, 1H), 4.79 (d, J=49.8 Hz, 1H), 4.57 (d, J=5.9 Hz, 2H), 3.90-3.80 (m, 2H), 3.65 (d, J=29.5 Hz, 1H), 3.08-2.98 (m, 1H), 2.79 (d, J=11.5 Hz, 1H), 2.33-2.15 (m, 4H), 2.12-2.04 (m, 1H), 2.00-1.90 (m, 1H), 1.62 (d, J=13.2 Hz, 6H).

### Example 202 and 203

### 2-(2-(3-((4-(dimethylphosphoryl)-2-methoxyphenyl)amino)prop-1-yn-1-yl)-7-(((3S,4R)-3-fluoro-1-methylpiperidin-4-yl)amino)benzo[b]thiophen-3-yl)acrylonitrile (202) and 2-(2-(3-((4-(dimethylphosphoryl)-2-methoxyphenyl)amino)prop-1-yn-1-yl)-7-(((3R,4S)-3-fluoro-1-methylpiperidin-4-yl)amino)benzo[b]thiophen-3-yl)acrylonitrile (203)

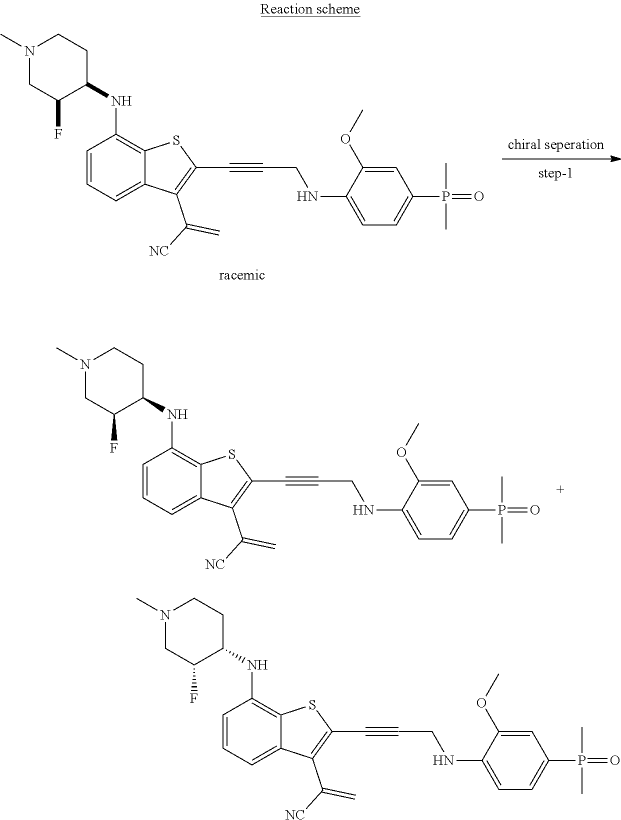

### Experimental Details

### Step 1. 2-(2-(3-((4-(dimethylphosphoryl)-2-methoxyphenyl)amino)prop-1-yn-1-yl)-7-(((3S,4R)-3-fluoro-1-methylpiperidin-4-yl)amino)benzo[b]thiophen-3-yl)acrylonitrile (202) and 2-(2-(3-((4-(dimethylphosphoryl)-2-methoxyphenyl)amino)prop-1-yn-1-yl)-7-(((3R,4S)-3-fluoro-1-methylpiperidin-4-yl)amino)benzo[b]thiophen-3-yl)acrylonitrile (203)

The racemic sample 2-(2-(3-((4-(dimethylphosphoryl)-2-methoxyphenyl)amino)prop-1-yn-1-yl)-7-(((Z)-3-fluoro-1-methylpiperidin-4-yl)amino)benzo[b]thiophen-3-yl)acrylonitrile (122.00 mg, 0.22 mmol) was separated by prep-chiral HPLC (Colunn:IG, Mobile Phase A: (HeX:DCM=3:1)(0.1% IPA.M), Mobile Phase B: EtOH; A:B (V:V=50:50); 220 nm; Flow rate: 20 mL/min; RT: 9.331 min) to provide 12.00 mg (9.83% yield) of 2-(2-(3-((4-(dimethylphosphoryl)-2-methoxyphenyl)amino)prop-1-yn-1-yl)-7-(((3R,4S)-3-fluoro-1-methylpiperidin-4-yl)amino)benzo[b]thiophen-3-yl)acrylonitrile (203) as off white solid. LCMS: m/z=551 [M+1]⁺

¹H NMR (400 MHz, DMSO-d₆) δ 7.35-7.29 (m, 1H), 7.29-7.19 (m, 1H), 7.19-7.10 (m, 2H), 6.83 (d, J=7.9 Hz, 2H), 6.61 (s, 1H), 6.40 (s, 1H), 6.09-6.04 (m, 1H), 5.39 (d, J=7.9 Hz, 1H), 4.83 (d, J=49.3 Hz, 1H), 4.33 (d, J=6.1 Hz, 2H), 3.87-3.79 (m, 3H), 3.69 (d, J=28.5 Hz, 1H), 3.10 (s, 1 H), 2.86 (s, 1H), 2.29 (d, J=31.8 Hz, 5H), 2.30-2.15 (m, 1H), 2.05-1.90 (m, 1H), 1.59 (d, J=13.2 Hz, 6H).

The racemic sample 2-(2-(3-((4-(dimethylphosphoryl)-2-methoxyphenyl)amino)prop-1-yn-1-yl)-7-(((Z)-3-fluoro-1-methylpiperidin-4-yl)amino)benzo[b]thiophen-3-yl)acrylonitrile (122.00 mg, 0.22 mmol) was separated by prep-chiral HPLC (Column:IG, Mobile Phase A: (HeX:DCM=3:1)(0.1% IPA.M)-Mobile Phase B: EtOH; A: B (V:V=50:50); 220 nm; Flow rate: 20 mL/min; RT: 10.966 min) to provide 17.00 mg (13.93% yield) of 2-(2-(3-((4-(dimethylphosphoryl)-2-methoxyphenyl)amino)prop-1-yn-1-yl)-7-(((3S,4R)-3-fluoro-1-methylpiperidin-4-yl)amino)benzo[b]thiophen-3-yl)acrylonitrile (202) as white solid. LCMS: m/z=551 [M+1]⁺

¹H NMR (400 MHz, DMSO-d₆) δ 7.35-7.28 (m, 1H), 7.26-7.20 (m, 1H), 7.19-7.13 (m, 2H), 6.90-6.73 (m, 2H), 6.61 (s, 1H), 6.40 (s, 1H), 6.09-6.01 (m, 1H), 5.35 (d, J=8.4 Hz, 1H), 4.91-4.70 (m, 1H), 4.34-4.20 (m, 2H), 3.85 (s, 3H), 3.75-3.65 (m, 1H), 3.10-3.00 (m, 1H), 2.81 (d, J=10.0 Hz, 1H), 2.33-2.25 (m, 1H), 2.19 (s, 3H), 2.15-2.05 (m, 1H), 2.04-1.90 (m, 1H), 1.75-1.65 (m, 1H), 1.59 (d, J=13.2 Hz, 6H).

### Example 204

### (5-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-1H-pyrazol-3-yl)dimethylphosphine oxide (204)

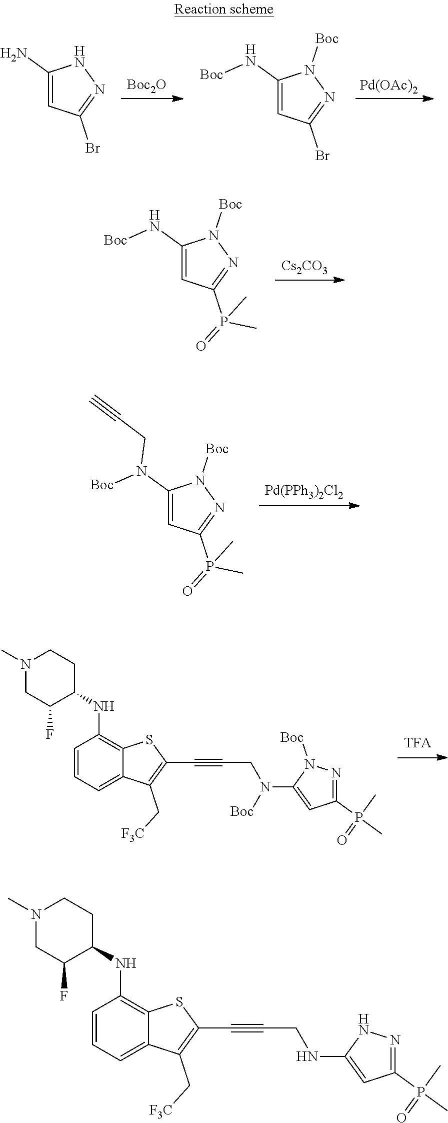

### Experimental Details

### Step 1. tert-butyl 3-bromo-5-((tert-butoxycarbonyl)amino)-1H-pyrazole-1-carboxylate

Into a 100-mL three necked bottle purged and maintained with an inert atmosphere of nitrogen, was placed 3-bromo-1H-pyrazol-5-amine (0.804 g, 4.96 mmol), Boc₂O (2.185 g, 10.01 mmol), TEA (1.015 g, 10.03 mmol), DMAP (0.072 g, 0.59 mmol), DCM (20 mL). The reaction mixture was stirred at 25° C. for 16 h. LCMS showed that the reaction was completed. The resulting solution was added to water (30 mL). The resulting solution was extracted with DCM (2×30 mL). The organic layers combined and washed with brine (20 mL), dried over anhydrous Na₂SO₄, the residue was concentrated under vacuum. The crude product purified by washing with HEX/EA (v/v=1/1). This resulted in 1.106 g (61.52% yield) of tert-butyl 3-bromo-5-((tert-butoxycarbonyl)amino)-1H-pyrazole-1-carboxylate as white solid. LCMS: m/z=362 [M+1]⁺.

### Step 2. tert-butyl 5-((tert-butoxycarbonyl)amino)-3-(dimethylphosphoryl)-1H-pyrazole-1-carboxylate

Into a 100-mL three necked bottle purged and maintained with an inert atmosphere of nitrogen, was placed tert-butyl 3-bromo-5-((tert-butoxycarbonyl)amino)-1H-pyrazole-1-carboxylate (0.922 g, 2.55 mmol), Pd(OAc)₂ (0.621 g, 2.77 mmol), Xantphos (0.519 g, 0.89 mmol), DIEA (0.882 g, 6.82 mmol) and DMF (20 mL). The reaction mixture was stirred at 130° C. for 0.5 h. Then was added Dimethylphosphine oxide (0.891 g, 11.42 mmol). The reaction mixture was stirred at 130° C. for another 1 h. LCMS showed that the reaction was completed. The reaction mixture was purified by C18 column eluted with ACN/H₂O (v/v=2/8). This resulted in 0.69 g (75.43% yield) of tert-butyl 5-((tert-butoxycarbonyl)amino)-3-(dimethylphosphoryl)-1H-pyrazole-1-carboxylate as red solid. LCMS: m/z=360 [M+1]⁺.

### Step 3. tert-butyl 5-((tert-butoxycarbonyl)(prop-2-yn-1-yl)amino)-3-(dimethylphosphoryl)-1H-pyrazole-1-carboxylate

Into a 50-mL flask was placed tert-butyl 5-((tert-butoxycarbonyl)amino)-3-(dimethylphosphoryl)-1H-pyrazole-1-carboxylate (0.471 g, 1.31 mmol), 3-Bromopropyne (0.464 g, 3.90 mmol), Cs₂CO₃ (0.470 g, 1.44 mmol), KI (0.224 g, 1.35 mmol), DMF (5 mL). The reaction mixture was stirred at 80° C. for 4 h. LCMS showed that the reaction was completed. The reaction mixture was purified by C18 column eluted with ACN/H₂O (v/v=2/8). This resulted in 0.036 g (9.40% yield) of tert-butyl 5-((tert-butoxycarbonyl)(prop-2-yn-1-yl)amino)-3-(dimethylphosphoryl)-1H-pyrrazole-1-carboxylate as red solid. LCMS: m/z=398 [M+1]⁺.

### Step 4. tert-butyl 5-((tert-butoxycarbonyl)(3-(7-(((3R,4S)-3-fluoro-1-methylpiperidin-4-yl)amino)-3-(2,2,2-trifluoroethyl)benzo[b]thiophen-2-yl)prop-2-yn-1-yl)amino)-3-(dimethylphosphoryl)-1H-pyrazole-1-carboxylate

Into a 50-mL three necked bottle and maintained with an inert atmosphere of nitrogen, was placed (3R,4S)-3-fluoro-N-(2-iodo-3-(2,2,2-trifluoroethyl)benzo[b]thiophen-7-yl)-1-methylpiperidin-4-amine (0.035 g, 0.074 m mol), tert-butyl 5-((tert-butoxycarbonyl)(prop-2-yn-1-yl)amino)-3-(dimethylphosphoryl)-1H-pyrazole-1-carboxylate (0.036 g, 0.091 mmol), CuI (0.025 g, 0.13 mmol), Pd(PPh₃)₂Cl₂ (0.024 g, 0.030 mmol), DIEA (0.026 g, 0.21 mmol) and DMSO (2 mL). The reaction mixture was stirred at room temperature for 1 h. LCMS showed that the reaction was completed. The reaction mixture was purified by C18 column eluted with ACN/H₂O (v/v=4/6). This resulted in 0.033 g (60.03% yield) of tert-butyl 5-((tert-butoxycarbonyl)(3-(7-(((3R,4S)-3-fluoro-1-methylpiperidin-4-yl)amino)-3-(2,2,2-trifluoroethyl)benzo[b]thiophen-2-yl)prop-2-yn-1-yl)amino)-3-(dimethylphosphoryl)-1H-pyrazole-1-carboxylate as red oil. LCMS: m/z=742 [M+1]⁺.

### Step 5. (5-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-1H-pyrazol-3-yl)dimethylphosphine oxide (204)

Into a 50-mL flask was placed tert-butyl 5-((tert-butoxycarbonyl)(3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-(dimethylphosphoryl)-1H-pyrazole-1-carboxylate (0.045 g, 0.061 mmol), DCM (2 mL), TFA (1 mL). The mixture was stirred at 20° C. for 1 h. LCMS showed that the reaction was completed. The resulting solution was added to sodium carbonate aqueous solution (50 mL) and extracted with EA (3×50 mL). The organic layers combined and washed with brine (50 mL), dried over anhydrous Na₂SO₄, the residue was concentrated under vacuum. The mixture was purified by preparative HPLC (Mobile Phase A: water (10 mmoL/L ammonium hydroxide), Mobil e Phase B: ACN; Flow rate: 40 mL/min; Gradient: 20-50-54% B (2-30-60 min); 262 nm; RT: 22.89-23.64 min). This resulted in 1.45 mg (4.41% yield) of (5-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-1H-pyrazol-3-yl)dimethylphosphine oxide (204) as off white solid. LCMS: m/z=542 [M+1]⁺.

### Example 205

### (4-((3-(7-((1-(3-fluoropropyl)piperidin-4-yl)amino)-3-(2,2,2-trifluoroethyl)benzo[b]thiophen-2-yl)prop-2-yn-1-yl)amino)-3-methoxyphenyl)dimethylphosphine oxide (205)

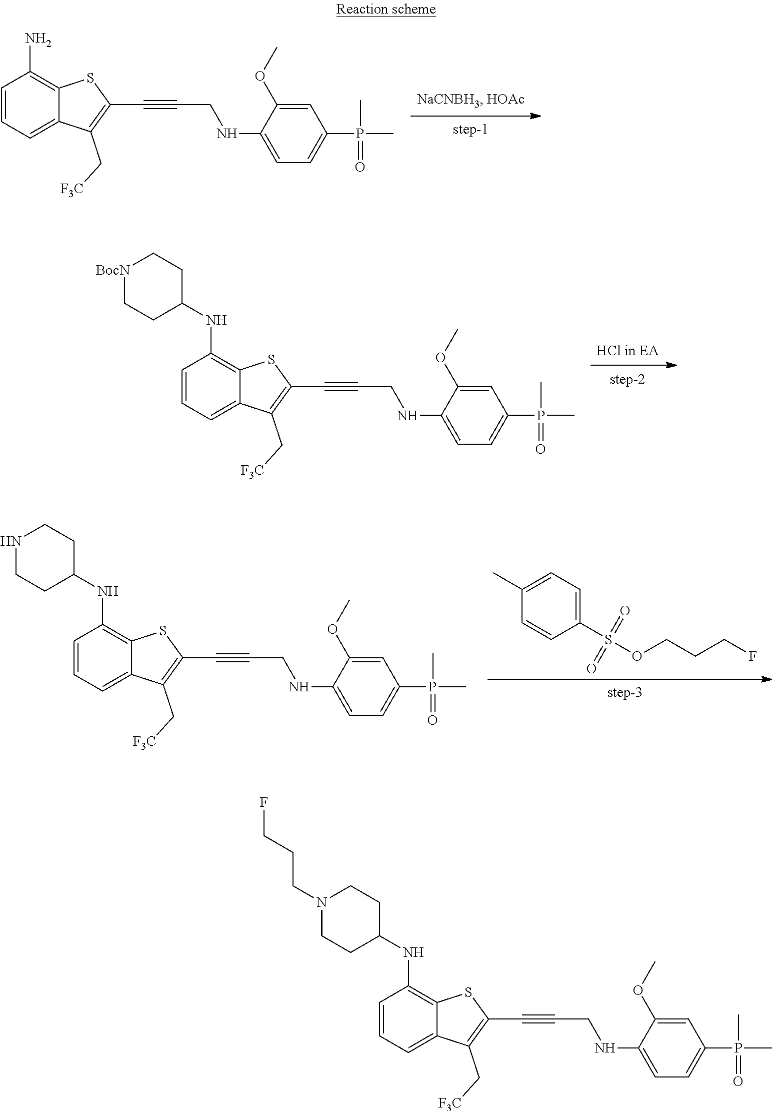 [SMILES: COc1cc(P(C)(C)=O)ccc1NCC#Cc1sc2c(N)cccc2c1CC(F)(F)F ; COc1cc(P(C)(C)=O)ccc1NCC#Cc1sc2c(NC3CCN(C(=O)OC(C)(C)C)CC3)cccc2c1CC(F)(F)F ; COc1cc(P(C)(C)=O)ccc1NCC#Cc1sc2c(NC3CCNCC3)cccc2c1CC(F)(F)F ; Cc1ccc(S(=O)(=O)OCCCF)cc1 ; COc1cc(P(C)(C)=O)ccc1NCC#Cc1sc2c(NC3CCN(CCCF)CC3)cccc2c1CC(F)(F)F]

### Experimental Details

### Step 1. tert-butyl 4-((2-(3-((4-(dimethylphosphoryl)-2-methoxyphenyl)amino)prop-1-yn-1-yl)-3-(2,2,2-trifluoroethyl)benzo[b]thiophen-7-yl)amino)piperidine-1-carboxylate

Into a 50-mL sealed tube purged and maintained with an inert atmosphere of nitrogen, was placed N-(tert-butoxycarbonyl)-4-piperidone (0.259 g, 1.30 mmol), (4-((3-(7-amino-3-(2,2,2-trifluoroethyl)benzo[b]thiophen-2-yl)prop-2-yn-1-yl)amino)-3-methoxyphenyl)dimethylphosphine oxide (0.202 g, 433.05 μmol), sodium cyanoboronhydride (0.085 g, 1.98 mmol), methanol (10 mL), acetic acid (0.1 mL). The reaction mixture was stirred at 50 l)benzo[b]thiophen-2-yl)prop-2-yn-1-yl)amino)-3-methoxyphenyl)dimethylphosphine oxide ophen-7-yl)amino)piperidine-1-carboxylate)-mbined, washed with brine (20 mL), dried over anhydrous Na₂SO₄ and concentrated under vacuum. The residues were purified by C₁₈ chromatography column eluted with ACN/H₂O (0.05% TFA) (v/v=1/1). This resulted in 0.200 g (71.08% yield) of tert-butyl4-((2-(3-((4-(dimethylphosphoryl)-2-methoxyphenyl)amino)prop-1-yn-1-yl)-3-(2,2,2-trifluoroethyl)benzo[b]thiophen-7-yl)amino)piperidine-1-carboxylate as white solid. LCMS: m/z=650 [M+1]⁺.

### Step 2. (3-methoxy-4-((3-(7-(piperidin-4-ylamino)-3-(2,2,2-trifluoroethyl)benzo[b]thiophen-2-yl)prop-2-yn-1-yl)amino)phenyl)dimethylphosphine oxide

Into a 20-mL round-bottom flask purged and maintained with an inert atmosphere of nitrogen, was placed tert-butyl4-((2-(3-((4-(dimethylphosphoryl)-2-methoxyphenyl)amino)prop-1-yn-1-yl)-3-(2,2,2-trifluoroethyl)benzo[b]thiophen-7-yl)amino)piperidine-1-carboxylate (0.208 g, 320.15 μmol), ethyl acetate (5 mL) and HCl in eth yl acetate solution (5 mL, 4 M). The reaction mixture was stirred at room temperature for 1.0 h. LCMS showed that the reaction was completed. Then saturated sodium hydrogen carbonate aqueous solution was added to the mixture until pH=7-8. The resulting solution was extracted with EA (2×10 mL). The organic layer was combined, washed with brine (10 mL), dried over anhydrous Na₂SO₄ and concentrated under vacuum. This resulted in 0.216 g (122.76% yield) of (3-methoxy-4-((3-(7-(piperidin-4-ylamino)-3-(2,2,2-trifluoroethyl)benzo[b]thiophen-2-yl)prop-2-yn-1-yl)amino)phenyl)dimethylphosphine oxide (crude) as yellow solid. LCMS: m/z=550 [M+1]⁺.

### Step 3. (4-((3-(7-((1-(3-fluoropropyl)piperidin-4-yl)amino)-3-(2,2,2-trifluoroethyl)benzo[b]thiophen-2-yl)prop-2-yn-1-yl)amino)-3-methoxyphenyl)dimethylphosphine oxide (205)

Into a 4-mL sealed tube purged and maintained with an inert atmosphere of nitrogen, was placed 3-fluoropropyl 4-methylbenzenesulfonate (0.047 g, 202.35 μmol), (3-methoxy-4-((3-(7-(piperidin-4-ylamino)-3-(2,2,2-trifluoroethyl)benzo[b]thiophen-2-yl)prop-2-yn-1-yl)amino)phenyl)dimethylphosphine oxide (0.101 g, 183.77 μmol), N,N-diisopropylethylamine (0.076 g, 588.04 μmol). The reaction mixture was stirred at 70)-3-(2,2,2-trifluoroethyl)benzo[b]thiophen-2-yl)prop-2-yn-1-yl)amino)phenyl)dimethylphosphine oxideenyl)dimethylphosphine oxidep-2-yn-1-yl)amino)phenyl)dimethylphosphin over anhydrous Na₂SO₄ and concentrated under vacuum. The mixture was purified by preparative HPLC (Mobile Phase A: water (10 mmoL/L ammonium hydroxide), Mobile Phase B: ACN; Flow rate: 70 mL/min; Gradient: 35-72-85% B (2-30-43 min; 266 nm; RT: 37.78-40.92 min). This resulted in 41.18 mg (36.75% yield) of (4-((3-(7-((1-(3-fluoropropyl)piperidin-4-yl)amino)-3-(2,2,2-trifluoroethyl)benzo[b]thiophen-2-yl)prop-2-yn-1-yl)amino)-3-methoxyphenyl)dimethylphosphine oxide (205) as white solid. LCMS: m/z=610[M+1]⁺.

¹H NMR (400 MHz, DMSO) δ 7.29-7.18 (m, 2H), 7.14 (d, J=10.0 Hz, 2H), 6.88-6.81 (m, 1H), 6.66 (d, J=7.8 Hz, 1H), 6.07 (t, J=6.2 Hz, 1H), 5.32 (d, J=8.0 Hz, 1H), 4.54 (t, J=6.0 Hz, 1H), 4.42 (t, J=5.9 Hz, 1H), 4.33 (d, J=6.3 Hz, 2H), 3.85 (s, 3H), 3.83-3.73 (m, 2H), 2.86 (d, J=9.0 Hz, 2H), 2.38 (s, 2H), 2.08-1.95 (m, 2H), 1.95-1.87 (m, 2H), 1.86-1.74 (m, 2H), 1.60 (s, 3H), 1.56 (s, 3H), 1.55-1.47 (m, 2H), 1.23 (s, 1H).

### Example 206

### (3-fluoro-4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-5-methoxyphenyl)dimethylphosphine oxide (206)

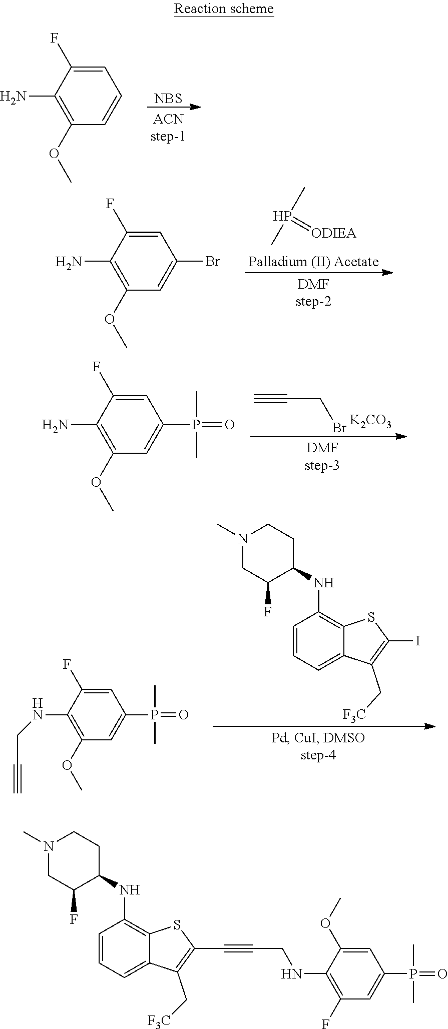

### Experiment Details

### Step 1 4-bromo-2-fluoro-6-methoxyaniline

Into a 50 ml 3-necked flask was placed 2-fluoro-6-methoxyaniline (3.12 g, 22.10 mmol), NBS (4.18 g, 23.48 mmol), DMF (30 mL). The reaction was stirred at rt for 2 h. The reaction mixture was concentrated under reduced pressure to afford crude product. The resulting solution was added to water (50 mL). The resulting solution was extracted with EA (3×100 mL). The organic layers combined and washed with brine (50 mL), dried over anhydrous Na₂SO₄, the residue was concentrated under vacuum. The crude product was purified with silica gel column eluted with EA/hexane (v/v=1/4). This resulted in 4.412 g, (90.70% yield) of 4-bromo-2-fluoro-6-methoxyaniline as yellow solid. LCMS: m/z=220 [M+1]⁺.

### Step 2 (4-amino-3-fluoro-5-methoxyphenyl)dimethylphosphine oxide

Into a 25 ml 3-necked flask was placed 4-bromo-2-fluoro-6-methoxyaniline (1.525 g, 6.93 mmol), Palladium (II) Acetate (0.321 g, 1.42 mmol), Xantphos (0.744 g, 1.28 mmol), DIEA (2.722 g, 21.06 mmol), DMF (15 mL) The reaction was stirred under nitrogen atmosphere at 130° C. for 0.5 h. Then dimethylphosphine oxide (2.023 g, 25.91 mmol) was added at 130° C.3mThe reaction was stirred under nitrogen atmosphere at 130° C. for 1 h The reaction mixture was filtered through celite pad and celite pad was washed with ACN (3×20 mL). The filterate was then concentrated under reduced pressure to afford the crude product. The crude product was purified by C18 column eluted with ACN/H₂O (v/v=1/7), filtered and concentrated under vacuum. This resulted in 1.278 g, (84.90% yield) of (4-amino-3-fluoro-5-methoxyphenyl)dimethylphosphine oxide as brown oil. LCMS: m/z=218 [M+1]⁺.

### Step 3 (3-fluoro-5-methoxy-4-(prop-2-yn-1-ylamino)phenyl)dimethylphosphine oxide

Into a 25 ml 3-necked flask was placed (4-amino-3-fluoro-5-methoxyphenyl)dimethylphosphine oxide (1.136 g, 5.23 mmol), 3-bromoprop-1-yne (2.385 g, 20.04 mmol), K₂CO₃ (1.492 g, 10.79 mmol), KI (1.054 g, 6.34 mmol) DMF (20 mL). The reaction mixture was stirred under nitrogen at 80° C. for 7 h. The reaction mixture was filtered through celite pad and celite pad was washed with ACN (3×20 mL). The reaction mixture was purified by C18 column eluted with ACN/H₂O (v/v=1/4), filtered and concentrated under vacuum. This resulted in 0.339 g, (25.39% yield) of (3-fluoro-5-methoxy-4-(prop-2-yn-1-ylamino)phenyl)dimethylphosphine oxide as brown oil. LCMS: m/z=256 [M+1]⁺.

### Step 4 (3-fluoro-4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-5-methoxyphenyl)dimethylphosphine oxide (206)

Into a 10-mL round-bottom flask was placed (3S,4R)-3-fluoro-N-(2-iodo-3-(2,2,2-trifluoroethyl)benzo[b]thiophen-7-yl)-1-methylpiperidin-4-amine (0.087 g, 184.21 umol), (3-fluoro-5-methoxy-4-(prop-2-yn-1-ylamino)phenyl)dimethylphosphine oxide (0.107 g, 419.23 umol), Pd(PPh₃)₂Cl₂ (0.028 g, 39.66 μmol), CuI (0.026 g, 136.51 umol), DIEA (0.091 g, 704.02 umol), methyl sulfoxide (2 mL). The reaction was stirred under nitrogen atmosphere at RT for 16 h. The reaction mixture was filtered through celite pad and celite pad was washed with ACN (3×20 mL). The crude product was purified by preparative HPLC (Mobile Phase A: water (0.1% ammonium hydroxide), Mobile Phase B: MeOH; Flow rate: 70 mL/min; Gradient: 35-68-72% B (2-30-34 min); 262 nm; RT: 30.32-32.47) to provide the desired product. This resulted in 0.080 g (72.42% yield) of (3-fluoro-4-((3-(7-(((3S,4R)-3-fluoro-1-methylpiperidin-4-yl)amino)-3-(2,2,2-trifluoroethyl)benzo[b]thiophen-2-yl) prop-2-yn-1-yl)amino)-5-methoxyphenyl)dimethylphosphine oxide (206) as white solid. LCMS: m/z=600 [M+1]⁺.

¹H NMR (400 MHz, DMSO-d₆) δ 7.31-7.08 (m, 4H), 6.77 (d, J=7.6 Hz, 1H), 5.72-5.69 (m, 1H), 5.13 (d, J=8.5 Hz, 1H), 4.79 (d, J=49.5 Hz, 1H), 4.39 (d, J=7.4 Hz, 2H), 3.88 (s, 3H), 3.62-6.59 (m, 3H), 3.03 (t, J=11.7 Hz, 1H), 2.79 (d, J=11.0 Hz, 1H), 2.18 (s, 4H), 2.07 (t, J=11.5 Hz, 1H), 1.95-1.92 (m, 1H), 1.77-1.69 (m, 1H), 1.62 (d, J=13.4 Hz, 6H).

### Example 207

### (6-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-5-methoxypyridin-3-yl)dimethylphosphine oxide (207)

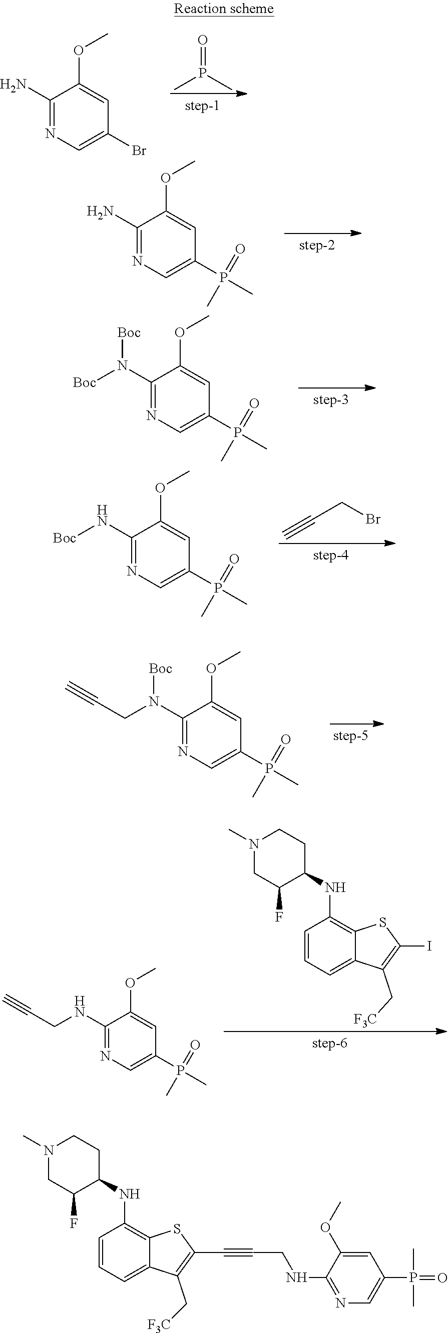

### Experiment Details

### Step 1. (6-amino-5-methoxypyridin-3-yl)dimethylphosphine oxide

Into a 50 mL three round bottom flask was placed 5-bromo-3-methoxypyridin-2-amine (1.02 g, 5.01 mmol), Palladium (II) acetate (0.21 g, 0.93 mmol), Dimethylbisdiphenylphosphinoxanthene (0.64 g, 1.11 mmol), DIEA (2.03 g, 15.67 mmol), DMF (10 mL). The reaction was heat to 130° C. under nitrogen. Dimethylphosphine oxide (1.17 g, 14.99 mmol) was added to the reaction slowly. The mixture was stirred at 130° C. for 1 h. The residue purified by flash-C18 eluted with ACN/water (V/V=1/9) to provide the desired product. This resulted in 0.580 g (57.85% yield) of (6-amino-5-methoxypyridin-3-yl)dimethylphosphine oxide as light-yellow solid. LCMS: m/z=201 [M+1]⁺.

### Step 2. tert-butyl N-tert-butoxycarbonyl-N-(5-dimethylphosphoryl-3-methoxy-2-pyridyl)carbamate

Into a 100 mL round-bottom flask was placed (6-amino-5-methoxypyridin-3-yl)dimethylphosphine oxide (0.58 g, 2.87 mmol), Di-tert-butyl dicarbonate (2.48 g, 11.35 mmol), N-(4-pyridyl)dimethylamine (0.20 g, 1.63 mmol), 1,4-Dioxane (30 mL). The reaction was stirred at 110° C. overnight. The mixture was concentrated under vacuum. This resulted in 1.016 g (crude) of tert-butyl N-tert-butoxycarbonyl-N-(5-dimethylphosphoryl-3-methoxy-2-pyridyl)carbamate as oil. LCMS: m/z=401 [M+1]⁺.

### Step 3. tert-butyl (5-(dimethylphosphoryl)-3-methoxypyridin-2-yl)carbamate

Into a 250 mL round-bottom flask was placed tert-butyl N-tert-butoxycarbonyl-N-(5-dimethylphosphoryl-3-methoxy-2-pyridyl)carbamate (3.85 g, 9.62 mmol), K₂CO₃ (4.18 g, 30.24 mmol), MeOH (50 mL). The reaction was stirred at 70° C. for 1 h. The mixture was concentrated under vacuum. The reaction was quenched by water (100 mL), extracted with EA (2×50 mL). The organic layers were combined, washed with brine (2×100 mL), dried over anhydrous sodium sulfate, filtered and concentrated under vacuum. The residue purified by flash-C18 eluted with ACN/water (v/v=3/7) to provide the desired product. This resulted in 1.275 g (44.16% yield) of tert-butyl (5-(dimethylphosphoryl)-3-methoxypyridin-2-yl)carbamate as brown solid. LCMS: m/z=301 [M+1]⁺.

### Step 4. tert-butyl (5-(dimethylphosphoryl)-3-methoxypyridin-2-yl)(prop-2-yn-1-yl)carbamate

Into a 250 mL round-bottom flask was placed tert-butyl (5-(dimethylphosphoryl)-3-methoxypyridin-2-yl)carba mate (1.21 g, 4.04 mmol), THF (50 mL). The reaction was stirred at 0° C. NaH (0.91 g, 38.00 mmol) was added to the reaction slowly at 0° C. and stirred for 0.5 h. 3-bromoprop-1-yne (9.45 g, 79.42 mmol) was added to the reaction and stirred at 60° C. for 2 h. The reaction was quenched by water (5 mL) and concentrated under vacuum. The residue purified by flash-C18 eluted with ACN/water (v/v=3/7) to provide the desired product. This resulted in 0.994 g (72.79% yield) of tert-butyl (5-(dimethylphosphoryl)-3-methoxypyridin-2-yl)(prop-2-yn-1-yl)carbamate as brown solid. LCMS: m/z=339 [M+1]⁺.

### Step 5. (5-methoxy-6-(prop-2-yn-1-ylamino)pyridin-3-yl)dimethylphosphine oxide

Into a 50 mL round-bottom flask was placed tert-butyl (5-(dimethylphosphoryl)-3-methoxypyridin-2-yl)(prop-2-yn-1-yl)carbamate (0.99 g, 2.94 mmol), TFA (5 mL), DCM (20 mL). The reaction was stirred at R.T for 0.5 h. The reaction was concentrated under vacuum. The residue purified by flash-C18 eluted with ACN/water (v/v=1/4) to provide the desired product. This resulted in 0.353 g (50.44% yield) of (5-methoxy-6-(prop-2-yn-1-ylamino)pyridin-3-yl)dimethylphosphine oxide as brown solid. LCMS: m/z=239 [M+1]⁺.

### Step 6. (6-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-5-methoxypyridin-3-yl)dimethylphosphine oxide (207)

Into a 20 mL vial was placed (3S,4R)-3-fluoro-N-(2-iodo-3-(2,2,2-trifluoroethyl)benzo[b]thiophen-7-yl)-1-meth ylpiperidin-4-amine (0.15 g, 0.32 mmol), (5-methoxy-6-(prop-2-yn-1-ylamino)pyridin-3-yl)dimethylphosphine oxide (0.09 g, 0.36 mmol), bis(triphenylphosphine)palladium(II) chloride (0.05 g, 75.09 μmol), CuI (0.02 g, 78.76 μmol), DIEA (0.35 g, 2.67 mmol), Methyl sulfoxide (5 mL). The reaction was stirred at 60° C. for 3 h under nitrogen. The reaction was quenched with water (30 mL), extracted with EA (2×20 mL). The organic layers were combined, washed with brine (2×50 mL), dried over anhydrous sodium sulfate, filtered and concentrated under vacuum. The mixture was purified by preparative HPLC (Mobile Phase A: water (10 mmol/L NH₄OH), Mobile Phase B: ACN; Flow rate: 70 mL/min; Gradient: 30-62-66% B (2-30-34 min); 266 nm; RT: 25.55-27.69 min) to provide the desired product. This resulted in 0.0083 g (4.46% yield) of (6-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-5-methoxypyridin-3-yl)dimethylphosphine oxide (207) as yellow solid. LCMS: m/z=583 [M+1]⁺.

¹H NMR (400 MHz, DMSO-d₆) δ 8.03-7.96 (m, 1H), 7.30-7.16 (m, 4H), 6.78 (d, J=7.7 Hz, 1H), 5.17 (d, J=8.5 Hz, 1H), 4.80 (d, J=49.5 Hz, 1H), 4.45 (d, J=6.0 Hz, 2H), 3.84 (m, 5H), 3.65 (d, J=28.5 Hz, 1H), 3.03 (t, J=11.3 Hz, 1H), 2.80 (d, J=10.9 Hz, 1H), 2.18 (m, 4H), 2.12-2.03 (m, 1H), 2.01-1.89 (m, 1H), 1.72 (d, J=12.5 Hz, 1H), 1.63 (d, J=13.3 Hz, 6H).

### Example 208

### (5-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-4-methoxypyrimidin-2-yl)dimethylphosphine oxide (208)

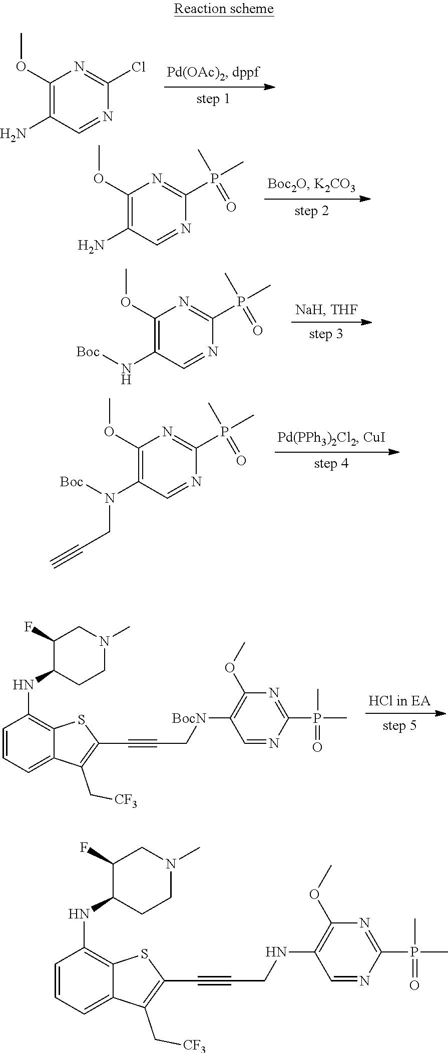

### Experiment Details

### Step 1. (5-amino-4-methoxypyrimidin-2-yl)dimethylphosphine oxide

Into a 100 mL flask purged and maintained with N₂ was placed 2-chloro-4-methoxypyrimidin-5-amine (1.055 g, 6.61 mmol), Palladium (II) acetate (0.141 g, 628.04 μmol), dppf (1.276 g, 2.26 mmol), DIPEA (0.351 g, 2.72 mmol), DMF (40 mL). The reaction was heated to 1201Hand dimethylphosphine oxide (0.678 g, 8.69 mmol) in DMF (2 mL) was added to the solution while stirring. The solution was stirred for 2 h. The solution was cooled to room temperature, and poured to water 20 mL. The solid was filtered, and the filter cake was washed with water 5 mL. The filtration was concentrate under vacuum and purified with C₁₈ column, eluted with ACN/water (v/v=1/4) to afford (5-amino-4-methoxypyrimidin-2-yl)dimethylphosphine oxide. LCMS: m/z=202[M+1]⁺.

### Step 2. tert-butyl (2-(dimethylphosphoryl)-4-methoxypyrimidin-5-yl)carbamate

Into a 25 mL flask was placed (5-amino-4-methoxypyrimidin-2-yl)dimethylphosphine oxide (0.197 g, 979.31 μmol), Boc₂O (0.558 g, 2.56 mmol), DCM (3 mL). The reaction was stirred overnight at room temperature. The reaction was concentrated. The residue was dissolved with MeOH (2 mL), and K₂CO₃ (0.053 g, 383.49 μmol) was added to the solution. The reaction was stirred for 1.5 h at room temperature then concentrated. The residue was purified with C₁₈ column, eluted with ACN/water (v/v=1/1) to afford tert-butyl (2-(dimethylphosphoryl)-4-methoxypyrimidin-5-yl)carbamate (0.279 g, 926.05 μmol, 94.56% yield) as off-white solid. LCMS: m/z=302[M+1]⁺.

### Step 3. tert-butyl (2-(dimethylphosphoryl)-4-methoxypyrimidin-5-yl)(prop-2-yn-1-yl)carbamate

Into a 20-mL three necked bottle purged and maintained with an inert atmosphere of nitrogen, was placed tert-butyl (2-(dimethylphosphoryl)-4-methoxypyrimidin-5-yl)carbamate (0.206 g, 683.75 μmol), tetrahydrofuran (5 mL). The reaction mixture was stirred at 0° C. Then sodium hydride (0.193 g, 8.04 mmol) was added. The mixture was stirred at 20° C. for 1.0 h. Then 3-bromopropyne (0.902 g, 7.58 mmol) was added to the reaction at −10° C. The reaction mixture was stirred at 70° C. for 1.0 h. The resulting solution was added to water (5 mL). The resulting solution was extracted with EA (2×10 mL). The organic layer was combined and washed with brine (10 mL), dried over anhydrous Na₂SO₄, the residue was concentrated under vacuum. The residue was purified by C₁₈ chromatography column eluted with ACN/H₂O (v/v=2/5). This resulted in 0.071 g (30.60% yield) of tert-butyl (2-(dimethylphosphoryl)-4-methoxypyrimidin-5-yl)(prop-2-yn-1-yl) carbamate as yellow oil. LCMS: m/z=340 [M+1]⁺.

### Step 4. tert-butyl (2-(dimethylphosphoryl)-4-methoxypyrimidin-5-yl)(3-(7-(((3S,4R)-3-fluoro-1-methylpiperidin-4-yl)amino)-3-(2,2,2-trifluoroethyl)benzo[b]thiophen-2-yl)prop-2-yn-1-yl)carbamate

Into a 4-mL three necked bottle purged and maintained with an inert atmosphere of nitrogen, was placed (3S,4R)-3-fluoro-N-(2-iodo-3-(2,2,2-trifluoroethyl)benzo[b]thiophen-7-yl)-1-methylpiperidin-4-amine (0.080 g, 169.39 μmol), tert-butyl (2-(dimethylphosphoryl)-4-methoxypyrimidin-5-yl)(prop-2-yn-1-yl)carbamate (0.058 g, 170.93 μmol), [1,1′-bis(diphenylphosphino)ferrocene]dichloropalladium(II) (0.020 g, 28.33 μmol), cuprous iodide (0.002 g, 10.50 μmol), T triethylamine (0.033 g, 326.12 μmol), methyl sulfoxide (1 mL). The reaction mixture was stirred at 50 refor 3 h. LCMS showed that the reaction was completed. The residue was purified by C₁₈ chromatography column eluted with ACN/H₂O (v/v=4/5). This resulted in 0.045 g (38.86% yield) of tert-butyl (2-(dimethylphosphoryl)-4-methoxypyrimidin-5-yl)(3-(7-(((3S,4R)-3-fluoro-1-methylpiperidin-4-yl)amino)-3-(2,2,2-trifluoroethyl)benzo[b]thiophen-2-yl)prop-2-yn-1-yl)carbamate as brown oil. LCMS: m/z=684 [M+1]⁺.

### Step 5. (5-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-4-methoxypyrimidin-2-yl)dimethylphosphine oxide (208)

Into a 25-mL round-bottom flask purged and maintained with an inert atmosphere of nitrogen, was placed tert-butyl (2-(dimethylphosphoryl)-4-methoxypyrimidin-5-yl)(3-(7-(((3S,4R)-3-fluoro-1-methylpiperidin-4-yl)amino)-3-(2,2,2-trifluoroethyl)benzo[b]thiophen-2-yl)prop-2-yn-1-yl)carbamate (0.045 g, 65.82 μmol), ethyl acetate (4 mL) and HCl in ethyl acetate solution (5 mL, 4 M). The reaction mixture was stirred at room temperature for 1.0 h. LCMS showed that the reaction was completed. Then saturated sodium hydrogen carbonate aqueous solution was added to the mixture until pH=7-8. The resulting solution was extracted with eth yl acetate (2×10 mL). The organic layer was combined, washed with brine (10 mL), dried over anhydrous Na₂SO₄ and concentrated under vacuum. The mixture was purified by preparative HPLC (Mobile Phase A: water (10 mmoL/L ammonium hydroxide), Mobile Phase B: ACN; Flow rate: 40 mL/min; Gradient: 20-52-52% B (2-30-31 min; 262 nm; RT: 27.13-27.86 min). This resulted in 0.010 g (26.03% yield) of (5-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-4-methoxypyrimidin-2-yl)dimethylphosphine oxide (208) as white solid. LCMS: m/z=584 [M+1]⁺.

¹H NMR (400 MHz, DMSO) δ 8.11 (s, 1H), 7.27 (t, J=7.9 Hz, 1H), 7.21 (d, J=7.9 Hz, 1H), 6.78 (d, J=7.5 Hz, 1H), 6.56 (t, J=6.2 Hz, 1H), 5.21 (d, J=8.3 Hz, 1H), 4.90-4.72 (m, 1H), 4.40 (d, J=6.5 Hz, 2H), 4.02 (s, 3H), 3.89-3.77 (m, 2H), 3.61 (s, 2H), 3.11-2.99 (m, 1H), 2.79 (d, J=9.5 Hz, 1H), 2.34-2.24 (m, 1H), 2.17 (d, J=10.0 Hz, 3H), 2.11-2.04 (m, 1H), 2.01-1.90 (m, 1H), 1.75-1.63 (m, 6H).

### Example 209

### (5-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-6-methoxypyrazin-2-yl)dimethylphosphine oxide (209)

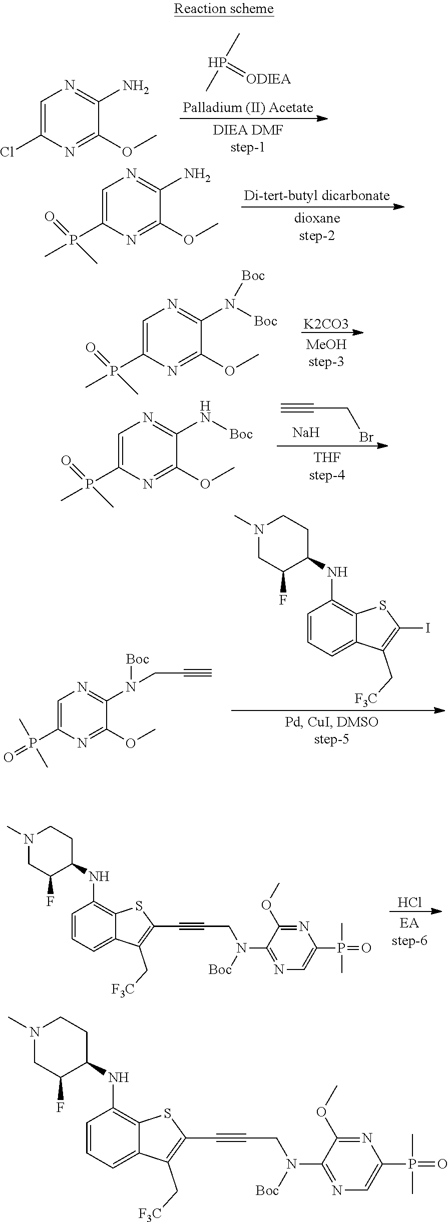

### Experiment Details

### Step 1 (5-amino-6-methoxypyrazin-2-yl)dimethylphosphine oxide

Into a 25 ml 3-necked flask was placed 5-chloro-3-methoxypyrazin-2-amine (0.943 g, 5.90 mmol), Palladium (II) Acetate (0.28 g, 1.26 mmol), DPPF (0.625 g, 1.15 mmol), DIEA (2.703 g, 20.91 mmol), DMF (10 mL) The reaction was stirred under nitrogen atmosphere at 130° C. for 0.5 h. Then dimethylphosphine oxide (1.429 g, 18.30 mmol) was added at 130° C. The reaction was stirred under nitrogen atmosphere at 130° C. for 1 h. The reaction mixture was filtered through celite pad and celite pad was washed with ACN (3×20 mL). The filterate was then concentrated under reduced pressure to afford the crude product. The reaction mixture was purified by C18 column eluted with ACN/H₂O (v/v=1/7), filtered and concentrated under vacuum. This resulted in 0.571 g, (48.03% yield) of (7-amino-2,2-difluorobenzo[d][1,3]dioxol-4-yl)dimethylphosphine oxide as yellow solid. LCMS: m/z=202 [M+1]⁺.

### Step 2 tert-butyl N-tert-butoxycarbonyl-N-(5-dimethylphosphoryl-3-methoxy-pyrazin-2-yl)carbamate

Into a 25 ml 3-necked flask was placed (7-amino-2,2-difluorobenzo[d][1,3]dioxol-4-yl)dimethylphosphine oxide (0.300 g, 1.49 mmol), Di-tert-butyl dicarbonate (1.649 g, 7.55 mmol), dioxane (5 mL). The reaction mixture was stirred under nitrogen at 100° C. for 16 h. The reaction mixture was concentrated under reduced pressure to afford crude product. The crude product was purified with silica gel column eluted with methano 1/dichloromethane (v/v=1/9). This resulted in 0.419 g, (69.99% yield) of tert-butyl N-tert-butoxycarbonyl-N-(5-dimethylphosphoryl-3-methoxy-pyrazin-2-yl)carbamate as yellow oil. LCMS: m/z=402 [M+1]⁺.

### Step 3 tert-butyl (5-(dimethylphosphoryl)-3-methoxypyrazin-2-yl)carbamate

Into a 25 ml 3-necked flask was placed tert-butyl N-tert-butoxycarbonyl-N-(5-dimethylphosphoryl-3-methoxy-pyrazin-2-yl)carbamate (0.399 g, 994.03 umol), K₂CO₃ (0.436 g, 3.15 mmol), MeOH (5 mL). The reaction mixture was stirred under nitrogen at 60° C. for 2 h. The reaction mixture was filtered through celite pad and celite pad was washed with ACN (3×20 mL). The filterate was then concentrated under reduced pressure to afford the crude product. The crude product was purified with silica gel column eluted with methanol/dichloromethane (v/v=1/5). This resulted in 0.215 g, (71.79% yield) of tert-butyl (5-(dimethylphosphoryl)-3-methoxypyrazin-2-yl)carbamate as yellow oil. LCMS: m/z=302 [M+1]⁺.

### Step 4. tert-butyl (5-(dimethylphosphoryl)-3-methoxypyrazin-2-yl)(prop-2-yn-1-yl)carbamate

Into a 25-mL three necked bottle purged and maintained with an inert atmosphere of nitrogen, was placed tert-butyl (5-(dimethylphosphoryl)-3-methoxypyrazin-2-yl)carbamate (0.129 g, 428.17 umol) and THF (3 mL). The reaction mixture was stirred at 0° C. Then NaH (0.098 g, 4.08 mmol) was added. The mixture was stirred at 0° C. for 1 h. 3-bromoprop-1-yne (0.400 g, 3.36 mmol) was added to the reaction at 0° C. The reaction mixture was stirred at 70° C. for 10 h. The resulting solution was added to MeOH (1 mL). The reaction mixture was filtered through celite pad and celite pad was washed with ACN (3×20 mL). The filterate was then concentrated under reduced pressure to afford the crude product. The crude product was purified with silica gel column eluted with methanol/dichloromethane (v/v=1/9). This resulted in 0.081 g, (55.75% yield) of tert-butyl (5-(dimethylphosphoryl)-3-methoxypyrazin-2-yl)(prop-2-yn-1-yl)carbamate as yellow oil. LCMS: m/z=340 [M+1]⁺.

### Step 5 tert-butyl (5-(dimethylphosphoryl)-3-methoxypyrazin-2-yl)(3-(7-(((3S,4R)-3-fluoro-1-methylpiperidin-4-yl)amino)-3-(2,2,2-trifluoroethyl)benzo[b]thiophen-2-yl)prop-2-yn-1-yl)carbamate

Into a 10-mL round-bottom flask was placed (3S,4R)-3-fluoro-N-(2-iodo-3-(2,2,2-trifluoroethyl)benzo[b]thiophen-7-yl)-1-methylpiperidin-4-amine (0.050 g, 105.86 umol), tert-butyl (5-(dimethylphosphoryl)-3-methoxypyrazin-2-yl)(prop-2-yn-1-yl)carbamate (0.069 g, 203.34 umol), Pd(PPh₃)₂Cl₂ (0.023 g, 32.58 μmol), CuI (0.014 g, 73.51 umol), DIEA (0.060 g, 464.24 umol), methyl sulfoxide (2 mL). The reaction was stirred under nitrogen atmosphere at RT for 16 h. The reaction mixture was filtered through celite pad and celite pad was washed with ACN (3×20 mL). The filterate was then concentrated under reduced pressure to afford the crude product. The crude product was purified by C18 column eluted with ACN/H₂O (v/v=2/3), filtered and concentrated under vacuum. This resulted in 0.056 g (77.36% yield) of tert-butyl (5-(dimethylphosphoryl)-3-methoxypyrazin-2-yl)(3-(7-(((3S,4R)-3-fluoro-1-methylpiperidin-4-yl)amino)-3-(2,2,2-trifluoroethyl)benzo[b]thiophen-2-yl)prop-2-yn-1-yl)carbamate as yellow solid. LCMS: m/z=684 [M+1]⁺.

### Step 6 (5-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-6-methoxypyrazin-2-yl)dimethylphosphine oxide (209)

Into a 10-mL round-bottom flask purged and maintained with an inert atmosphere of nitrogen, was placed tert-butyl (5-(dimethylphosphoryl)-3-methoxypyrazin-2-yl)(3-(7-(((3S,4R)-3-fluoro-1-methylpiperidin-4-yl)amino)-3-(2,2,2-trifluoroethyl)benzo[b]thiophen-2-yl)prop-2-yn-1-yl)carbamate (0.041 g, 59.96 umol), ethyl acetate (1 mL) and HCl in EA solution (1 mL, 4 M). The reaction mixture was stirred at room temperature for 1 h. The mixture was concentrated to afford the crude product. The crude product was purified by preparative HPLC (Mobile Phase A: water (0.1% ammonium hydroxide), Mobile Phase B: ACN; Flow rate: 40 mL/min; Gradient: 20-44-48% B (2-28-34 min); 262 nm; 31.77-33.23) to provide the desired product. This resulted in 0.025 g (71.43% yield) of (5-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-6-methoxypyrazin-2-yl)dimethylphosphine oxide (209) as white solid. LCMS: m/z=584 [M+1]⁺.

¹H NMR (400 MHz, DMSO-d₆) δ 8.03 (d, J=2.3 Hz, 1H), 7.88 (t, J=5.8 Hz, 1H), 7.31-7.18 (m, 2H), 6.78 (d, J=7.7 Hz, 1H), 5.15 (d, J=8.6 Hz, 1H), 4.88-4.73 (m, 1H), 4.46 (d, J=5.8 Hz, 2H), 3.96 (s, 3H), 3.84-3.81 (m, 2H), 3.65 (d, J=29.0 Hz, 1H), 3.03 (t, J=11.1 Hz, 1H), 2.79 (d, J=11.1 Hz, 1H), 2.27 (d, J=13.0 Hz, 1H), 2.18 (s, 3H), 2.08 (t, J=11.4 Hz, 1H), 1.95-1.91 (m, 1H), 1.79-1.69 (m, 1H), 1.60 (d, J=13.2 Hz, 6H).

### Example 210

### (4-((3-(7-(((3S,4R)-3-fluoro-1-(methyl-d3)piperidin-4-yl)amino)-3-(2,2,2-trifluoroethyl)benzo[b]thiophen-2-yl) prop-2-yn-1-yl)amino)-3-methoxyphenyl)dimethylphosphine oxide (210)

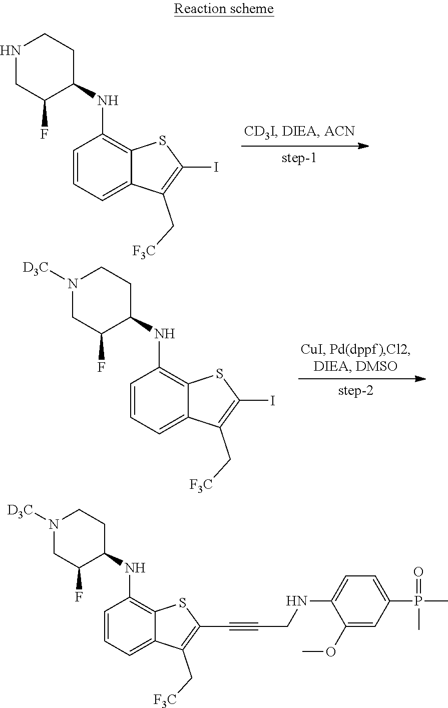

### Experiment Details

### Step 1. (3S,4R)-3-fluoro-N-(2-iodo-3-(2,2,2-trifluoroethyl)benzo[b]thiophen-7-yl)-1-(methyl-d3)piperidin-4-amine

Into a 8-mL sealed tube purged and maintained with an inert atmosphere of nitrogen, was placed (3S,4R)-3-fluoro-N-(2-iodo-3-(2,2,2-trifluoroethyl)benzo[b]thiophen-7-yl)piperidin-4-amine (0.110 g, 0.24 mmol), Trideuteromethyl iodide (0.040 g, 0.27), DIEA (0.086 g, 0.67 mmol), ACN (5 mL). The reaction mixture was stirred at room temperature for 1 h. The mixture was purified with C18 column eluted with ACN/water (v/v=1/1) to afford (3S,4R)-3-fluoro-N-(2-iodo-3-(2,2,2-trifluoroethyl)benzo[b]thiophen-7-yl)-1-(methyl-d₃)piperidin-4-amine (0.083 g, 72.75% yield) as yellow oil. LCMS: m/z=476 [M+1]⁺

### Step 2. (4-((3-(7-(((3S,4R)-3-fluoro-1-(methyl-d3)piperidin-4-yl)amino)-3-(2,2,2-trifluoroethyl)benzo[b]thiophen-2-yl)prop-2-yn-1-yl)amino)-3-methoxyphenyl)dimethylphosphine oxide (210)

Into a 8-mL sealed tube purged and maintained with an inert atmosphere of nitrogen, was placed (3S,4R)-3-fluoro-N-(2-iodo-3-(2,2,2-trifluoroethyl)benzo[b]thiophen-7-yl)-1-(methyl-d3)piperidin-4-amine (0.101 g, 0.21 mmol), (3-methoxy-4-(prop-2-yn-1-ylamino)phenyl)dimethylphosphine oxide (0.76 g, 0.32 mmol), Pd(dppf)Cl₂ (0.024 g, 0.03 mmol), CuI (0.020 g, 0.11 mmol), DIEA (0.124 g, 0.96 mmol), DMSO (1 mL). The reaction mixture was stirred at 50° C. for 1 h. The mixture was purified by preparative HPLC (Mobile Phase A: water (10 mmoL/L ammonium hydroxide), Mobile Phase B: MeCN; Flow rate: 70 mL/min; Gradient: 40-75-95% B (2-32-60 min) 260 nm; RT: 31.40-33.96 min) to afford (4-((3-(7-(((3S,4R)-3-fluoro-1-(methyl-d3)piperidin-4-yl)amino)-3-(2,2,2-trifluoroethyl)benzo[b]thiophen-2-yl)prop-2-yn-1-yl)amino)-3-methoxyphenyl)dimeth ylphosphine oxide (210) (0.0561 g, 60.01% yield) as white solid. LCMS: m/z=585 [M+1]⁺

¹H NMR (400 MHz, DMSO) δ 7.30-7.18 (m, 3H), 7.14 (d, J=11.9 Hz, 1H), 6.90-6.80 (m, 1H), 6.78 (d, J=7.7 Hz, 1H), 6.10-5.99 (m, 1H), 5.16 (d, J=8.5 Hz, 1H), 4.79 (d, J=49.5 Hz, 1H), 4.34 (d, J=6.4 Hz, 2H), 3.85 (s, 3H), 3.83-3.72 (m, 2H), 3.64 (d, J=28.0 Hz, 1H), 3.06-2.99 (m, 1H), 2.79 (d, J=12.2 Hz, 1H), 2.30-2.13 (m, 1H), 2.08 (t, J=10.9 Hz, 1H), 2.00-1.87 (m, 1H), 1.79-1.66 (m, 1H), 1.59 (s, 3H), 1.56 (s, 3H).

### Example 211

### 5-(dimethylphosphoryl)-2-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)benzonitrile (211)

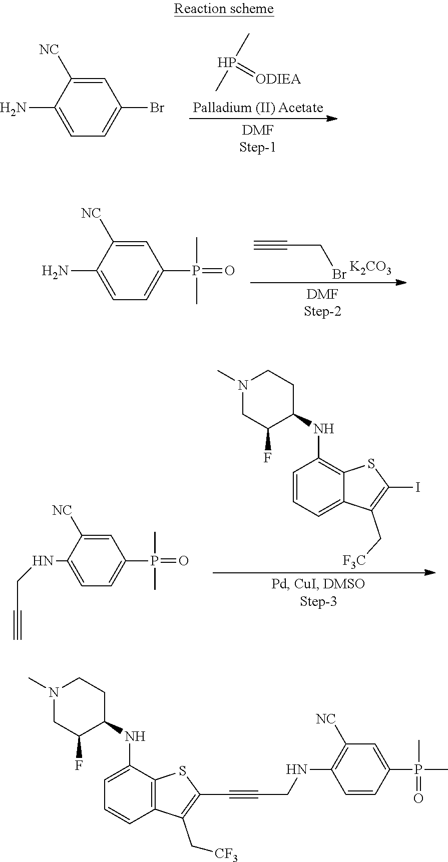

### Experiment Details

### Step 1 2-amino-5-(dimethylphosphoryl)benzonitrile

Into a 25 ml 3-necked flask was placed 2-amino-5-bromobenzonitrile (2.075 g, 10.53 mmol), Palladium (II) Acetate (0.372 g, 1.65 mmol), Xantphos (0.891 g, 1.53 mmol), DIEA (4.012 g, 31.04 mmol), DMF (20 m L) The reaction was stirred under nitrogen atmosphere at 130° C. for 0.5 h. Then dimethylphosphine oxide (2.98 g, 38.18 mmol) was added at 130° C.3. The reaction was stirred under nitrogen atmosphere at 130° C. for 1 h The reaction mixture was filtered through celite pad and celite pad was washed with ACN (3×20 mL). The filterate was then concentrated under reduced pressure to afford the crude product. The crude product was purified by C18 column eluted with ACN/H₂O (v/v=1/7), filtered and concentrated under vacuum. This resulted in 1.802 g, (88.12% yield) of 2-amino-5-(dimethylphosphoryl)benzonitrile as brown oil. LCMS: m/z=195 [M+1]⁺.

### Step 2 5-(dimethylphosphoryl)-2-(prop-2-yn-1-ylamino)benzonitrile

Into a 25 ml 3-necked flask was placed 2-amino-5-(dimethylphosphoryl)benzonitrile (0.560 g, 2.88 mmol), 3-bromoprop-1-yne (0.700 g, 5.88 mmol), DIEA (0.506 g, 3.91 mmol), DMF (10 mL). The reaction mixture was stirred under nitrogen at 60° C. for 56 h. The reaction mixture was filtered through celite pad and celite pad was washed with ACN (3×20 mL). The reaction mixture was purified by C18 column eluted with ACN/H₂O (v/v=1/4), filtered and concentrated under vacuum. This resulted in 0.106 g, (15.82% yield) of 5-(dimethylphosphoryl)-2-(prop-2-yn-1-ylamino)benzonitrile as brown oil. LCMS: m/z=233 [M+1]⁺.

### Step 3 5-(dimethylphosphoryl)-2-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)benzonitrile (211)

Into a 10-mL round-bottom flask was placed (3S,4R)-3-fluoro-N-(2-iodo-3-(2,2,2-trifluoroethyl)benzo[b]thiophen-7-yl)-1-methylpiperidin-4-amine (0.080 g, 169.39 umol), 5-(dimethylphosphoryl)-2-(prop-2-yn-1-ylamino)benzonitrile (0.096 g, 413.40 umol), Pd(PPh₃)₂Cl₂ (0.028 g, 39.66 μmol), CuI (0.015 g, 78.76 umol), DIEA (0.115 g, 889.80 umol), methyl sulfoxide (2 mL). The reaction was stirred under nitrogen atmosphere at RT for 16 h. The reaction mixture was filtered through celite pad and celite pad was washed with ACN (3×20 mL). The crude product was purified by preparative HPLC (Mobile Phase A: water (0.1% ammonium hydroxide), Mobile Phase B: MeOH; Flow rate: 70 mL/min; Gradient: 35-69-69% B (2-30-35 min); 266 nm; R T: 27.27-29.29) to provide the desired product. This resulted in 0.060 g (61.43% yield) of 5-(dimethylphosphoryl)-2-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)benzonitrile (211) as white solid. LCMS: m/z=577 [M+1]⁺.

¹H NMR (400 MHz, DMSO-d₆) δ 7.97-7.76 (m, 2H), 7.26-7.30 (m, 3H), 7.11 (d, J=8.7 Hz, 1H), 6.79 (d, J=7.6 Hz, 1H), 5.16 (d, J=8.4 Hz, 1H), 4.80 (d, J=49.5 Hz, 1H), 4.45 (d, J=5.9 Hz, 2H), 3.83-3.80 (m, 2H), 3.74-3.58 (m, 1H), 3.03 (t, J=11.3 Hz, 1H), 2.80 (d, J=10.9 Hz, 1H), 2.31-1.90 (m, 6H), 1.78-1.68 (m, 1H), 1.61 (d, J=13.3 Hz, 6H).

### Example 212

### 3-(5-(dimethylphosphoryl)-2-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)phenyl)-1,2,4-oxadiazol-5(2H)-one (212)

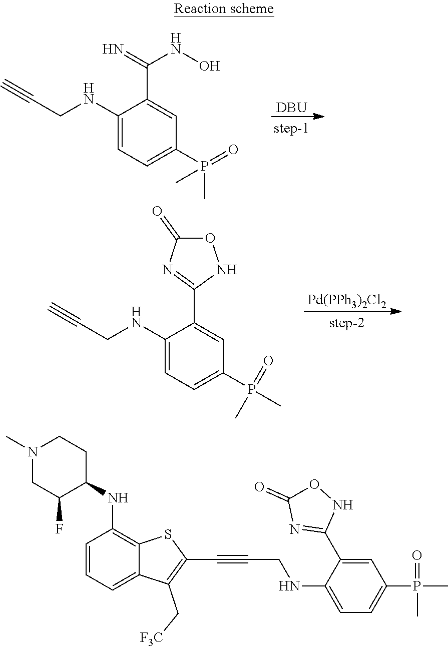

### Step 1. 3-(5-(dimethylphosphoryl)-2-(prop-2-yn-1-ylamino)phenyl)-1,2,4-oxadiazol-5(2H)-one

Into a 100-mL three necked bottle purged and maintained with an inert atmosphere of nitrogen, was placed 5-(dimethylphosphoryl)-N-hydroxy-2-(prop-2-yn-1-ylamino)benzimidamide (0.269 g, 1.01 mmol), CDI (0.512 g, 3.16 mmol), DBU (0.354 g, 2.33 mmol), DMF (3 mL). The reaction mixture was stirred at 100° C. for 2 h. LCMS showed that the reaction was completed. The reaction mixture was purified by C18 column eluted with ACN/H₂O (v/v=1/10). This resulted in 0.049 g (16.59% yield) of 3-(5-(dimethylphosphoryl)-2-(prop-2-yn-1-ylamino)phenyl)-1,2,4-oxadiazol-5(2H)-one as red solid. LCMS: m/z=292 [M+1]⁺.

### Step 2. 3-(5-(dimethylphosphoryl)-2-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)phenyl)-1,2,4-oxadiazol-5(2H)-one (212)

Into a 50-mL three necked bottle and maintained with an inert atmosphere of nitrogen, was placed (3S,4R)-3-fluoro-N-(2-iodo-3-(2,2,2-trifluoroethyl)benzo[b]thiophen-7-yl)-1-methylpiperidin-4-amine (0.059 g, 0.12 m mol), 3-(5-(dimethylphosphoryl)-2-(prop-2-yn-1-ylamino)phenyl)-1,2,4-oxadiazol-5(2H)-one (0.044 g, 0.15 mmol), CuI (0.070 g, 0.37 mmol), Pd(PPh₃)₂Cl₂ (0.021 g, 0.030 mmol), DIEA (0.070 g, 0.54 mmol) and DMSO (2 mL). The reaction mixture was stirred at room temperature for 1 h. LCMS showed that the reaction was completed. The mixture was purified by preparative HPLC (Mobile Phase A: water (10 mmoL/L ammonium hydroxide), Mobile Phase B: ACN; Flow rate: 40 mL/min; Gradient: 35-69-69% B (2-30-60 min); 266 nm; RT: 6.45-7.91 min). This resulted in 0.019 g (24.00% yield) of 3-(5-(dimethylphosphoryl)-2-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)phenyl)-1,2,4-oxadiazol-5(2H)-one (212) as off white solid. LCMS: m/z=636 [M+1]⁺.

¹H NMR (600 MHz, DMSO-d₆) δ 8.01 (dd, J=12.0, 1.9 Hz, 1H), 7.81 (s, 1H), 7.69 (dd, J=10.4, 8.3 Hz, 1H), 7.33-7.24 (m, 2H), 7.08 (dd, J=8.6, 2.1 Hz, 1H), 6.80 (d, J=7.6 Hz, 1H), 5.40 (d, J=8.3 Hz, 1H), 4.95 (d, J=48.3 Hz, 1H), 4.58 (d, J=5.8 Hz, 2H), 3.92-3.75 (m, 3H), 3.30 (s, 3H), 3.12 (s, 1H), 2.92-2.70 (m, 2H), 2.05 (t, J=12.5 Hz, 1H), 1.85 (d, J=13.3 Hz, 1H), 1.61 (d, J=13.1 Hz, 6H), 1.25 (d, J=8.0 Hz, 1H).

### Example 213

### (4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-(1,2,4-oxadiazol-3-yl)phenyl)dimethylphosphine oxide (213)

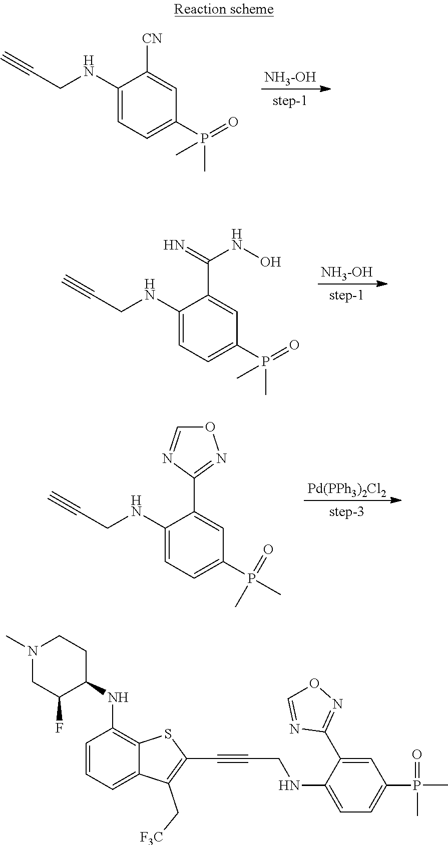

### Experimental Details

### Step 1. 5-(dimethylphosphoryl)-N-hydroxy-2-(prop-2-yn-1-ylamino)benzimidamide

Into a 100-mL three necked bottle purged and maintained with an inert atmosphere of nitrogen, was placed 5-(dimethylphosphoryl)-2-(prop-2-yn-1-ylamino)benzonitrile (0.88 g, 3.79 mmol), NH₃—OH (50% in water, 10 mL), EtOH (10 mL). The reaction mixture was stirred at 25° C. for 4 h. LCMS showed that the reaction was completed. The reaction mixture was purified by C18 column eluted with ACN/H₂O (v/v=1/10). This resulted in 0.982 g (97.89% yield) of 5-(dimethylphosphoryl)-N-hydroxy-2-(prop-2-yn-1-ylamino)benzimidamide as red solid. LCMS: m/z=266 [M+1]⁺.

### Step 2. (3-(1,2,4-oxadiazol-3-yl)-4-(prop-2-yn-1-ylamino)phenyl)dimethylphosphine oxide

Into a 100-mL three necked bottle was placed 5-(dimethylphosphoryl)-N-hydroxy-2-(prop-2-yn-1-ylamino)benzimidamide (0.366 g, 1.38 mmol), Trimethoxymethane (1 mL), AcOH (0.1 mL). The reaction mixture was stirred at 25° C. for 16 h. The reaction mixture was purified by C18 column eluted with ACN/H₂O (v/v=4/6). This resulted in 0.109 g (28.70% yield) of (3-(1,2,4-oxadiazol-3-yl)-4-(prop-2-yn-1-ylamino)phenyl)dimethylphosphine oxide as red oil. LCMS: m/z=276 [M+1].

### Step 3. (4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-(1,2,4-oxadiazol-3-yl)phenyl)dimethylphosphine oxide (213)

Into a 50-mL three necked bottle and maintained with an inert atmosphere of nitrogen, was placed (3S,4R)-3-fluoro-N-(2-iodo-3-(2,2,2-trifluoroethyl)benzo[b]thiophen-7-yl)-1-methylpiperidin-4-amine (0.058 g, 0.12 m mol), (3-(1,2,4-oxadiazol-3-yl)-4-(prop-2-yn-1-ylamino)phenyl)dimethylphosphine oxide (0.097 g, 0.35 mmol), CuI (0.114 g, 0.60 mmol), Pd(PPh₃)₂Cl₂ (0.035 g, 0.050 mmol), DIEA (0.114 g, 0.88 mmol) and DMSO (5 mL). The reaction mixture was stirred at room temperature for 1 h. LCMS showed that the reaction was completed. The mixture was purified by preparative HPLC (Mobile Phase A: water (10 mmoL/L ammonium hydroxide), Mobile Phase B: ACN; Flow rate: 40 mL/min; Gradient: 35-70-70% B (2-30-60 min); 266 nm; RT: 32.26-33.93 min). This resulted in 0.027 g (35.48% yield) of (4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-(1,2,4-oxadiazol-3-yl)phenyl)dimethylphosphine oxide (213) as off white solid. LCMS: m/z=620 [M+1]⁺.

¹H NMR (400 MHz, DMSO) δ 9.78 (s, 1H), 8.42 (d, J=11.9 Hz, 1H), 7.78 (t, J=9.5 Hz, 1H), 7.42 (t, J=5.8 Hz, 1H), 7.33-7.16 (m, 3H), 6.79 (d, J=7.6 Hz, 1H), 5.15 (d, J=8.4 Hz, 1H), 4.79 (d, J=49.6 Hz, 1H), 4.67 (d, J=5.7 Hz, 2H), 3.93-3.76 (m, 2H), 3.74-3.55 (m, 1H), 3.03 (t, J=10.4 Hz, 1H), 2.79 (d, J=10.9 Hz, 1H), 2.27 (d, J=12.8 Hz, 1H), 2.18 (s, 3H), 2.08 (t, J=11.3 Hz, 1H), 2.00-1.89 (m, 1H), 1.71 (d, J=11.7 Hz, 1H), 1.65 (s, 3H), 1.61 (s, 3H).

### Example 214

### (4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-(oxazol-5-yl)phenyl)dimethylphosphine oxide (214)

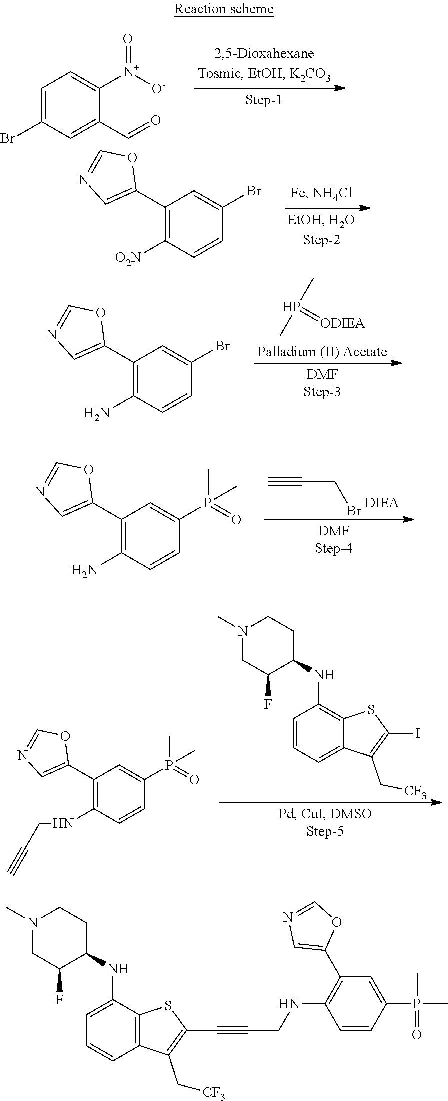

### Experiment Details

### Step 1 5-(5-bromo-2-nitrophenyl)oxazole

Into a 100 ml 3-necked flask was placed 5-bromo-2-nitrobenzaldehyde (5.15 g, 22.38 mmol), Tosylmethyl isocyanide (6.57 g, 33.65 mmol), K₂CO₃ (10.05 g, 72.71 mmol), 2,5-Dioxahexane (15 mL), EtOH. (30 mL). The reaction was stirred at 100 for 1 h. The reaction was quenched with water (50 mL). The resulted solution was extracted with EA (3×100 mL), washed with brine (50 mL), dried over anhydrous sodium sulfate, filtered and concentrated under vacuum. The crude product was purified with silica gel column eluted with EA/hexane (v/v=1/3). This resulted in 3.34 g (55.44% yield) of 5-(5-bromo-2-nitrophenyl)oxazole as yellow solid. LCMS: m/z=269 [M+1]⁺.

### Step 2 4-bromo-2-(oxazol-5-yl)aniline

Into a 100 ml 3-necked flask was placed 4-bromo-2-(fluoromethoxy)-1-nitrobenzene (3.30 g, 12.26 mmol), Iron (6.80 g, 121.76 mmol), NH₄Cl (6.73 g, 125.81 mmol), EtOH (30 mL), water (6 mL). The reaction was stirred at 95 for 2 h. The reaction mixture was filtered through celite pad and celite pad was washed with methanol (2×30 mL). The filterate was then concentrated under reduced pressure to afford the product. The crude product was purified with silica gel column eluted with EA/hexane (v/v=2/1). This resulted in 1.665 g (56.78% yield) of 44-bromo-2-(oxazol-5-yl)aniline as yellow solid. LCMS: m/z=239 [M+1]⁺.

### Step 3 (4-amino-3-(oxazol-5-yl)phenyl)dimethylphosphine oxide

Into a 25 ml 3-necked flask was placed 4-bromo-2-(oxazol-5-yl)aniline (1.635 g, 6.83 mmol), Palladium (II) Acetate (0.320 g, 1.42 mmol), Xantphos (0.744 g, 1.28 mmol), DIEA (2.517 g, 19.47 mmol), DMF (20 m L) The reaction was stirred under nitrogen atmosphere at 130° C. for 0.5 h. Then dimethylphosphine oxide (4.705 g, 60.28 mmol) was added at 130° C.3 The reaction was stirred under nitrogen atmosphere at 130° C. for 1 h The reaction mixture was purified by C18 column eluted with ACN/H₂O (v/v=1/9), filtered and concentrated under vacuum. This resulted in 1.823 g, (112.84% yield) of (4-amino-3-(oxazol-5-yl)phenyl)dimethylphosphine oxide as brown oil. LCMS: m/z=237 [M+1]⁺.

### Step 4 dimethyl(3-(oxazol-5-yl)-4-(prop-2-yn-1-ylamino)phenyl)phosphine oxide

Into a 25 ml 3-necked flask was placed (4-amino-3-(oxazol-5-yl)phenyl)dimethylphosphine oxide (0.556 g, 2.35 mmol), 3-bromoprop-1-yne (0.506 g, 4.25 mmol), DIEA (0.214 g, 1.65 mmol), DMF (10 mL). The reaction mixture was stirred under nitrogen at 60° C. for 6 h. The reaction mixture was filtered through celite p ad and celite pad was washed with ACN (3×20 mL). The reaction mixture was purified by C18 column eluted with ACN/H₂O (v/v=1/6), filtered and concentrated under vacuum. This resulted in 0.078 g, (12.08% yield) of dimethyl(3-(oxazol-5-yl)-4-(prop-2-yn-1-ylamino)phenyl)phosphine oxide as brown oil. LCMS: m/z=275 [M+1]⁺.

### Step 5 (4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-(oxazol-5-yl)phenyl)dimethylphosphine oxide (214)

Into a 10-mL round-bottom flask was placed (3S,4R)-3-fluoro-N-(2-iodo-3-(2,2,2-trifluoroethyl)benzo[b]thiophen-7-yl)-1-methylpiperidin-4-amine (0.079 g, 167.27 umol), dimethyl(3-(oxazol-5-yl)-4-(prop-2-yn-1-ylamino)phenyl)phosphine oxide (0.059 g, 215.12 umol), Pd(PPh₃)₂Cl₂ (0.022 g, 31.16 μmol), CuI (0.015 g, 78.76 umol), DIEA (0.102 g, 789.21 umol), methyl sulfoxide (2 mL). The reaction was stirred under nitrogen atmosphere at RT for 16 h. The reaction mixture was filtered through celite pad and celite pad was washed with ACN (3×20 mL). The filterate was then concentrated under reduced pressure to afford the crude product The crude product was purified by preparative HPLC (Mobile Phase A: water (0.1% ammonium hydroxide), Mobile Phase B: MeOH; Flow rate: 70 mL/min; Gradient: 35-67-67% B (2-30-33 min); 264 nm; RT: 27.40-29.39) to provide the desired product. This resulted in 0.064 g (61.84% yield) of (4-((3-(7-((3-fluoro-1-methylpiperidin-4-yl)amino)-3-(2,2,2-trifluoroethyl)benzo[b]thiophen-2-yl)prop-2-yn-1-yl)amino)-3-(fluoromethoxy)phenyl)dimethylphosphine oxide (214) as white solid. LCMS: m/z=619 [M+1]⁺.

¹H NMR (400 MHz, DMSO-d₆) δ 8.49 (s, 1H), 7.79 (d, J=11.5 Hz, 1H), 7.64 (t, J=9.6 Hz, 1H), 7.5 (s, 1H), 7.24-7.19 (m, 2H), 7.11-7.07 (m, 1H), 6.78 (d, J=7.6 Hz, 1H), 6.29 (t, J=6.2 Hz, 1H), 5.15 (d, J=8.5 Hz, 1H), 4.79 (d, J=49.5 Hz, 1H), 4.44 (d, J=5.9 Hz, 2H), 3.82-3.78 (m, 2H), 3.65 (d, J=29.1 Hz, 1H), 3.03 (t, J=11.3 Hz, 1H), 2.79 (d, J=11.0 Hz, 1H), 2.18 (s, 4H), 2.08 (t, J=11.5 Hz, 1H), 1.99-1.88 (m, 1H), 1.71 (d, J=12.8 Hz, 1H), 1.61 (d, J=13.2 Hz, 6H).

### Example 215

### N-cyclopropyl-5-(dimethylphosphoryl)-2-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)benzamide (215)

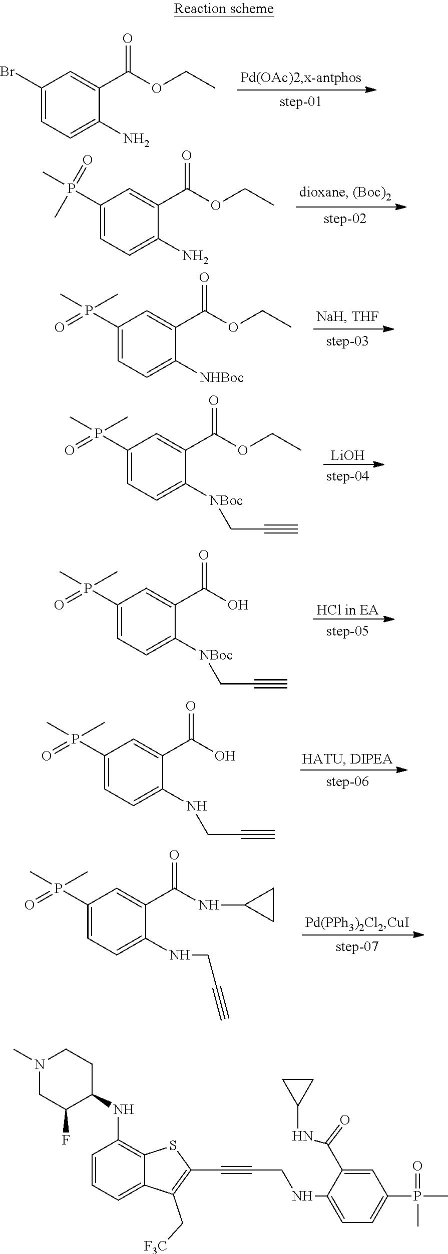

### Experimental Details

### Step 1. ethyl 2-amino-5-(dimethylphosphoryl)benzoate

Into a 100-mL three necked bottle purged and maintained with an inert atmosphere of nitrogen, was placed ethyl 2-amino-5-bromobenzoate (5.006 g, 20.51 mmol), palladium (II) acetate (0.773 g, 3.44 mmol), dimethylbisdiphenylphosphinoxanthene (1.896 g, 3.28 mmol), N,N-diisopropylethylamine (5.712 g, 44.20 mmol), N,N-dimethylformamide (20 mL). The reaction mixture was heated to 130° C. Then methylphosphinoylmethane (6.639 g, 85.06 mmol) was added and stirred at the same temperature for 1.0 h. LCMS showed that the reaction was completed. The residue was filtered and purified by C₁₈ chromatography column eluted with ACN/H₂O (0.05% NH₄HCO₃) (v/v=1/3). This resulted in 3.53 g (71.35% yield) of ethyl 2-amino-5-(dimethylphosphoryl)benzoate as yellow oil. LCMS: m/z=242 [M+1]⁺.

### Step 2. ethyl 2-((tert-butoxycarbonyl)amino)-5-(dimethylphosphoryl)benzoate

Into a 100-mL three necked bottle purged and maintained with an inert atmosphere of nitrogen, was placed ethyl 2-amino-5-(dimethylphosphoryl)benzoate (3.49 g, 14.47 mmol), di-tert-butyl dicarbonate (10.33 g, 47. 33 mmol), N-(4-pyridyl)dimethylamine (0.42 g, 3.44 mmol), N,N-diisopropylethylamine (9.58 g, 74.12 mmol), 1,4-dioxane (50 mL). The reaction mixture was stirred at 110° C. for 3 h. LCMS showed that the reaction was completed. The resulting solution was concentrated and purified by C₁₈ chromatography column eluted with ACN/H₂O (v/v=1/1). This resulted in 0.97 g (19.64% yield) of ethyl 2-((tert-butoxycarbonyl)amino)-5-(dimethylphosphoryl)benzoate as white solid. LCMS: m/z=342 [M+1]⁺.

### Step 3. ethyl 2-((tert-butoxycarbonyl)(prop-2-yn-1-yl)amino)-5-(dimethylphosphoryl)benzoate

Into a 25-mL three necked bottle purged and maintained with an inert atmosphere of nitrogen, was placed thyl 2-((tert-butoxycarbonyl)amino)-5-(dimethylphosphoryl)benzoate (1.002 g, 2.94 mmol), tetrahydrofuran (10 mL). The reaction mixture was stirred at 0° C. Then sodium hydride (0.677 g, 28.21 mmol) was added. The mixture was stirred at 20° C. for 1.0 h. Then 3-bromopropyne (3.683 g, 30.96 mmol) was added to the reaction at −10° C. The reaction mixture was stirred at room temperature for 1 h. The reaction was quenched by the addition of acetic acid (5 mL). The resulting solution was extracted with EA (2×20 mL). The organic layer was combined and washed with brine (50 mL), dried over anhydrous Na₂SO₄, the residue w as concentrated under vacuum. The residue was purified by C₁₈ chromatography column eluted with ACN/H₂O (v/v=1/1). This resulted in 1.082 g (97.15% yield) of ethyl 2-((tert-butoxycarbonyl)(prop-2-yn-1-yl)amino)-5-(dimethylphosphoryl)benzoate as yellow oil. LCMS: m/z=380 [M+1]⁺.

### Step 4. 2-((tert-butoxycarbonyl)(prop-2-yn-1-yl)amino)-5-(dimethylphosphoryl)benzoic acid

Into a 50-mL three necked bottle and maintained with an inert atmosphere of nitrogen, was placed lithium hydroxide (0.348 g, 14.53 mmol), ethyl 2-((tert-butoxycarbonyl) (prop-2-yn-1-yl)amino)-5-(dimethylphosphoryl)benzoate (1.022 g, 2.69 mmol), methanol (15 mL), water (5 mL). The reaction mixture was stirred at 50 refor 2 h. LCMS showed that the reaction was completed. Then saturated hydrochloric acid aqueous solution was added to the mixture until pH=4-5 and extracted with CHCl₃/IPA (v/v=3/1, 2×20 mL). The organic layer was combined and dried over anhydrous Na₂SO₄, the residue was concentrated under vacuum. This resulted in 0.78 g (82.42% yield) of 2-((tert-butoxycarbonyl)(prop-2-yn-1-yl)amino)-5-(dimethylphosphoryl)benzoic acid as yellow oil. LCMS: m/z=352 [M+1]⁺.

### Step 5. 5-(dimethylphosphoryl)-2-(prop-2-yn-1-ylamino)benzoic acid

Into a 50-mL round-bottom flask purged and maintained with an inert atmosphere of nitrogen, was placed 2-((tert-butoxycarbonyl)(prop-2-yn-1-yl)amino)-5-(dimethylphosphoryl)benzoic acid (0.77 g, 2.19 mmol), ethyl acetate (10 mL) and HCl in ethyl acetate solution (5 mL, 4 M). The reaction mixture was stirred at room temperature for 2 h. LCMS showed that the reaction was completed. Then DCM (2×10 mL) was added and concentrated under vacuum. This resulted in 0.85 g (154.38% yield) of 5-(dimethylphosphoryl)-2-(prop-2-yn-1-ylamino)benzoic acid(hydrochloride) as yellow oil. LCMS: m/z=252 [M+1]⁺.

### Step 6. N-cyclopropyl-5-(dimethylphosphoryl)-2-(prop-2-yn-1-ylamino)benzamide

Into a 4-mL round-bottom flask purged and maintained with an inert atmosphere of nitrogen, was placed 5-(dimethylphosphoryl)-2-(prop-2-yn-1-ylamino)benzoic acid(hydrochloride) (0.062 g, 246.80 μmol), 2-(7-aza-1H-benzotriazole-1-yl)-1,1,3,3-tetramethyluronium hexafluorophosphate (0.118 g, 310.34 μmol), cyclopropylamine (0.037 g, 648.05 μmol), N,N-diisopropylethylamine (0.241 g, 1.86 mmol), N,N-dimethylformamide (1 m L). The reaction mixture was stirred at room temperature for 1 h. LCMS showed that the reaction was completed. The residues were purified by C₁₈ chromatography column eluted with ACN/H₂O (v/v=1/5). This resulted in 0.60 g (83.75% yield) of N-cyclopropyl-5-(dimethylphosphoryl)-2-(prop-2-yn-1-ylamino)benzamide as yellow oil. LCMS: m/z=291 [M+1]⁺.

### Step 7. N-cyclopropyl-5-(dimethylphosphoryl)-2-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)benzamide (215)

Into a 8-mL flask purged and maintained with an inert atmosphere of nitrogen, was placed (3S,4R)-3-fluoro-N-(2-iodo-3-(2,2,2-trifluoroethyl)benzo[b]thiophen-7-yl)-1-methylpiperidin-4-amine (0.062 g, 131.28 μmol), N-cyclopropyl-5-(dimethylphosphoryl)-2-(prop-2-yn-1-ylamino)benzamide (0.041 g, 141.23 μmol), [1,1′-bis(diphenylphosphino)ferrocene]dichloropalladium(II) (0.017 g, 24.08 μmol), cuprous iodide (0.001 g, 5.25 μmol), triethylamine (0.044 g, 434.83 μmol), methyl sulfoxide (1 mL). The reaction mixture was stirred at 50 refor 24 h. LCMS showed that the reaction was completed. The resulting solution was quenched with water (10 mL) and extracted with EA (2×20 mL). The organic layer was combined and washed with brine (20 mL), dried over anhydrous Na₂SO₄, the residue was concentrated under vacuum. The mixture was purified by preparative HPLC (Mobile Phase A: water (10 mmoL/L ammonium hydroxide), Mobile Phase B: ACN; Flow rate: 40 mL/min; Gradient: 25-57-57% B (2-30-32 min; 266 nm; RT: 26.06-26.98 min). This resulted in 0.006 g (7.20% yield) of N-cyclopropyl-5-(dimethylphosphoryl)-2-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)benzamide (215) as white solid. LCMS: m/z=635 [M+1]⁺.

¹H NMR (400 MHz, DMSO) δ 8.53 (s, 1H), 8.37 (s, 1H), 7.80 (d, J=11.8 Hz, 1H), 7.70-7.61 (m, 1H), 7.32-7.25 (m, 1H), 7.22 (d, J=8.2 Hz, 1H), 6.99 (d, J=8.4 Hz, 1H), 6.79 (d, J=7.5 Hz, 1H), 5.15 (d, J=8.2 Hz, 1H), 4.88-4.72 (m, 1H), 4.47 (d, J=5.8 Hz, 2H), 3.89-3.78 (m, 2H), 3.65 (d, J=29.8 Hz, 2H), 3.03 (s, 2H), 2.81 (s, 2H), 2.19 (s, 3H), 1.61 (s, 3H), 1.58 (s, 3H), 1.23 (s, 2H), 0.71 (d, J=6.5 Hz, 2H), 0.59 (s, 2H).

### Example 216

### 5-(dimethylphosphoryl)-2-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-N-(tetrahydro-2H-pyran-4-yl)benzamide (216)

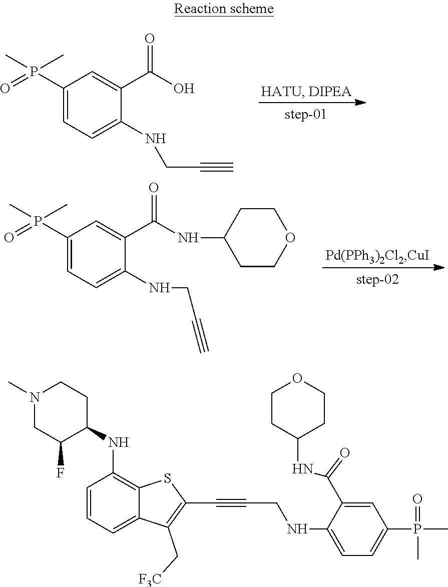

### Experimental Details

### Step 1. 5-(dimethylphosphoryl)-2-(prop-2-yn-1-ylamino)-N-(tetrahydro-2H-pyran-4-yl)benzamide

Into a 8-mL round-bottom flask purged and maintained with an inert atmosphere of nitrogen, was placed 5-(dimethylphosphoryl)-2-(prop-2-yn-1-ylamino)benzoic acid(hydrochloride) (0.102 g, 406.02 μmol), 4-aminotetrahydropyran (0.081 g, 800.81 μmol), N,N-diisopropylethylamine (0.172 g, 1.33 mmol), 2-(7-aza-1H-benzotriazole-1-yl)-1,1,3,3-tetramethyluronium hexafluorophosphate (0.500 g, 1.32 mmol), N,N-dimethylformamide (3 mL). The reaction mixture was stirred at room temperature for 4 h. LCMS showed that the reaction was completed. The residues were purified by C₁₈ chromatography column eluted with ACN/H₂O (v/v=1/3). This resulted in 0.094 g (69.24% yield) of 5-(dimethylphosphoryl)-2-(prop-2-yn-1-ylamino)-N-(tetrahydro-2H-pyran-4-yl)benzamide as yellow oil. LCMS: m/z=335 [M+1]⁺.

### Step 2. 5-(dimethylphosphoryl)-2-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-N-(tetrahydro-2H-pyran-4-yl)benzamide (216)

Into a 4-mL flask purged and maintained with an inert atmosphere of nitrogen, was placed (3S,4R)-3-fluoro-N-(2-iodo-3-(2,2,2-trifluoroethyl)benzo[b]thiophen-7-yl)-1-methylpiperidin-4-amine (0.060 g, 127.04 μmol), 5-(dimethylphosphoryl)-2-(prop-2-yn-1-ylamino)-N-(tetrahydro-2H-pyran-4-yl)benzamide (0.063 g, 188.43 μmol), [1,1′-bis(diphenylphosphino)ferrocene]dichloropalladium(II) (0.026 g, 36.83 μmol), cuprous iodide (0.004 g, 21.00 μmol), triethylamine (0.077 g, 760.95 μmol), meth yl sulfoxide (1 mL). The reaction mixture was stirred at 50 refor 25 h. LCMS showed that the reaction w as completed. The resulting solution was quenched with water (10 mL) and extracted with EA (2×20 mL). The organic layer was combined and washed with brine (20 mL), dried over anhydrous Na₂SO₄, the residue was concentrated under vacuum. The mixture was purified by preparative HPLC (Mobile Phase A: w ater (10 mmoL/L ammonium hydroxide), Mobile Phase B: ACN; Flow rate: 40 mL/min; Gradient: 20-50-54% B (2-30-34 min; 266 nm; RT: 30.80-31.96 min). This resulted in 0.028 g (32.47% yield) of 5-(dimethylphosphoryl)-2-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-N-(tetrahydro-2H-pyran-4-yl)benzamide (216) as white solid. LCMS: m/z=679 [M+1]⁺.

¹H NMR (400 MHz, DMSO) δ 8.41 (d, J=7.6 Hz, 1H), 8.26 (s, 1H), 7.86 (d, J=10.2 Hz, 1H), 7.70-7.62 (m, 1H), 7.30-7.20 (m, 2H), 7.02-6.96 (m, 1H), 6.79 (d, J=7.3 Hz, 1H), 5.14 (d, J=8.7 Hz, 1H), 4.88-4.70 (m, 1H), 4.46 (d, J=6.0 Hz, 2H), 4.01 (s, 2H), 3.93-3.75 (m, 5H), 3.03 (s, 2H), 2.78 (s, 2H), 2.35-2.25 (m, 2H), 2.18 (s, 3H), 2.08 (s, 1H), 1.95 (s, 1H), 1.80-1.73 (m, 3H), 1.63 (s, 3H), 1.60 (s, 3H).

### Example 217

### 5-(dimethylphosphoryl)-2-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-N-(oxetan-3-yl)benzamide (217)

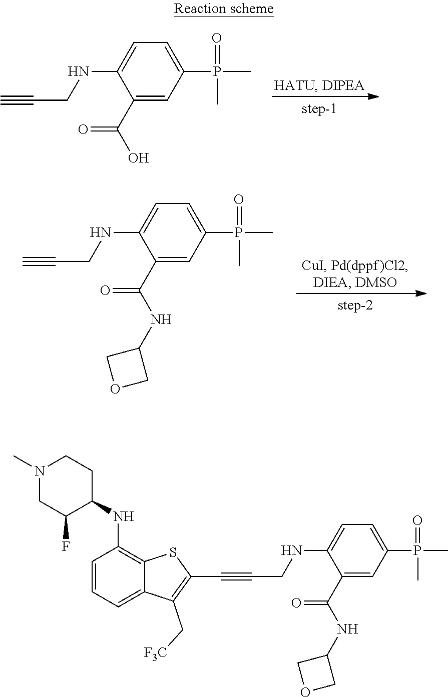

### Experiment Details

### Step 1. 5-(dimethylphosphoryl)-N-(oxetan-3-yl)-2-(prop-2-yn-1-ylamino)benzamide

Into a 8-mL sealed tube was placed 5-(dimethylphosphoryl)-2-(prop-2-yn-1-ylamino)benzoic acid (0.110 g, 0.46 mmol), oxetan-3-amine (0.063 g, 0.86 mmol), HATU (0.189 g, 0.50 mmol), DIEA (0.184 g, 1.42 mmol), DMF (1 mL). The reaction mixture was stirred at room temperature for 1 h. The mixture was purified with C18 column eluted with ACN/water (v/v=1/1) to afford 5-(dimethylphosphoryl)-N-(oxetan-3-yl)-2-(prop-2-yn-1-ylamino)benzamide (0.069 g, 48.79% yield) as yellow oil. LCMS: m/z=307 [M+1]⁺

### Step 2. 5-(dimethylphosphoryl)-2-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-N-(oxetan-3-yl)benzamide (217)

Into a 8-mL sealed tube purged and maintained with an inert atmosphere of nitrogen, was placed (3S,4R)-3-fluoro-N-(2-iodo-3-(2,2,2-trifluoroethyl)benzo[b]thiophen-7-yl)-1-methylpiperidin-4-amine (0.095 g, 0.20 mmol), 5-(dimethylphosphoryl)-N-(oxetan-3-yl)-2-(prop-2-yn-1-ylamino)benzamide (0.066 g, 0.22 mmol), Pd(dppf) Cl₂ (0.019 g, 0.03 mmol), CuI (0.006 g, 0.03 mmol), DIEA (0.079 g, 0.60 mmol), DMSO (1 mL). The reaction mixture was stirred at 50° C. for 1 h. The mixture was purified by preparative HPLC (Mobile Phase A: water (10 mmoL/L ammonium hydroxide), Mobile Phase B: MeCN; Flow rate: 70 mL/min; Gradient: 40-75-950% B (2-32-60 min) 260 nm; RT: 31.40-33.96 min) to afford (4-((3-(7-(((3S,4R)-3-fluoro-1-(methyl-d3)piperidin-4-yl)amino)-3-(2,2,2-trifluoroethyl)benzo[b]thiophen-2-yl)prop-2-yn-1-yl)amino)-3-methoxyphenyl)dimethylphosphine oxide (217) (0.0545 g, 41.64% yield) as white solid. LCMS: m/z=651 [M+1]⁺

¹H NMR (400 MHz, CD₃OD) δ 8.00 (d, J=12.3 Hz, 1H), 7.76 (t, J=9.5 Hz, 1H), 7.39-7.28 (m, 1H), 7.22 (d, J=8.0 Hz, 1H), 7.12 (d, J=8.5 Hz, 1H), 6.81 (d, J=7.6 Hz, 1H), 5.21-5.07 (m, 1H), 5.04-4.90 (m, 3H), 4.81-4.69 (m, 2H), 4.45 (s, 2H), 3.80-3.62 (m, 3H), 3.26-3.16 (m, 1H), 3.05-2.90 (m, 1H), 2.51-2.37 (m, 1H), 2.34 (s, 3H), 2.31-2.19 (m, 1H), 2.05-1.91 (m, 2H), 1.79 (s, 3H), 1.78 (s, 3H).

### Example 218

### 2-((3-(7-(((3S,4R)-3-chloro-1-methylpiperidin-4-yl)amino)-3-(2,2,2-trifluoroethyl)benzo[b]thiophen-2-yl)prop-2-yn-1-yl)amino)-5-(dimethylphosphoryl)-N,N-dimethylbenzamide (218)

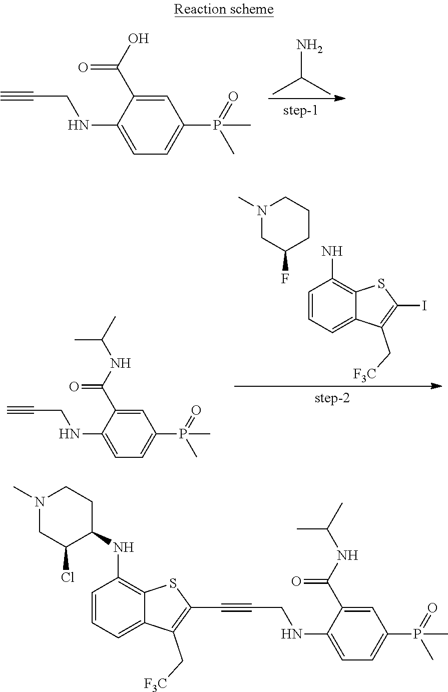

### Experiment Details

### Step 1. 5-(dimethylphosphoryl)-N,N-dimethyl-2-(prop-2-yn-1-ylamino)benzamide

Into a 8 mL vial was placed 5-(dimethylphosphoryl)-2-(prop-2-yn-1-ylamino)benzoic acid (0.10 g, 0.41 mmol), propan-2-amine (0.06 g, 0.96 mmol), HATU (0.22 g, 0.59 mmol), DIEA (0.17 g, 1.31 mmol), DMF (1 mL). The reaction was stirred at R.T for 2 h under nitrogen. The residue purified by flash-C18 eluted with ACN/water (v/v=3/7) to provide the desired product. This resulted in 0.077 g (64.88% yield) of 5-(dimethylphosphoryl)-N,N-dimethyl-2-(prop-2-yn-1-ylamino)benzamide as brown solid. LCMS: m/z=279 [M+1]⁺

### Step 2. 2-((3-(7-(((3S,4R)-3-chloro-1-methylpiperidin-4-yl)amino)-3-(2,2,2-trifluoroethyl)benzo[b]thiophen-2-yl)prop-2-yn-1-yl)amino)-5-(dimethylphosphoryl)-N,N-dimethylbenzamide (218)

Into a 20 mL vial was placed (3S,4R)-3-fluoro-N-(2-iodo-3-(2,2,2-trifluoroethyl)benzo[b]thiophen-7-yl)-1-meth ylpiperidin-4-amine (0.10 g, 0.21 mmol), 5-(dimethylphosphoryl)-N,N-dimethyl-2-(prop-2-yn-1-ylamino)benzamide (0.08 g, 0.26 mmol), bis(triphenylphosphine)palladium(II) chloride (0.03 g, 46.75 μmol), CuI (0.01 g, 52.51 μmol), DIEA (0.10 g, 0.77 mmol), Methyl sulfoxide (5 mL). The reaction was stirred at 60° C. for 2 h under nitrogen. The reaction was quenched with water (30 mL), extracted with EA (2×20 mL). The organic layers were combined, washed with brine (2×50 mL), dried over anhydrous sodium sulfate, filtered and concentrated under vacuum. The mixture was purified by preparative HPLC (Mobile Phase A: water (10 mmol/L ammonium hydroxide), Mobile Phase B: ACN; Flow rate: 70 mL/min; Gradient: 35-71-71% B (2-33-34 min); 264 nm; RT: 31.48-33.20 min) to provide the desired product. This resulted in 0.0503 g (36.94% yield) of 2-((3-(7-(((3S,4R)-3-chloro-1-methylpiperidin-4-yl)amino)-3-(2,2,2-trifluoroethyl)benzo[b]thiophen-2-yl)prop-2-yn-1-yl)amino)-5-(dimethylphosphoryl)-N,N-dimethylbenzamide (218) as white solid. LCMS: m/z=639 [M+1]⁺.

¹H NMR (600 MHz, DMSO) δ 8.36-8.27 (m, 2H), 7.85 (d, J=11.7 Hz, 1H), 7.65 (t, J=9.5 Hz, 1H), 7.31-7.25 (m, 1H), 7.25-7.19 (m, 1H), 7.01-6.97 (m, 1H), 6.79 (d, J=7.8 Hz, 1H), 5.17 (d, J=8.6 Hz, 1H), 4.87-4.72 (m, 1H), 4.47 (d, J=6.0 Hz, 2H), 4.16-4.07 (m, 1H), 3.90-3.80 (m, 2H), 3.66 (d, J=28.4 Hz, 1H), 3.09-2.99 (m, 1H), 2.80 (d, J=10.9 Hz, 1H), 2.31-2.22 (m, 1H), 2.19 (s, 3H), 2.12-2.05 (m, 1H), 2.02-1.90 (m, 1H), 1.77-1.68 (m, 1H), 1.63 (s, 3H), 1.61 (s, 3H), 1.20 (s, 3H), 1.18 (s, 3H).

### Example 219

### 2-((3-(7-(((3S,4R)-3-chloro-1-methylpiperidin-4-yl)amino)-3-(2,2,2-trifluoroethyl)benzo[b]thiophen-2-yl)prop-2-yn-1-yl)amino)-5-(dimethylphosphoryl)-N-(2-methoxyethyl)benzamide (219)

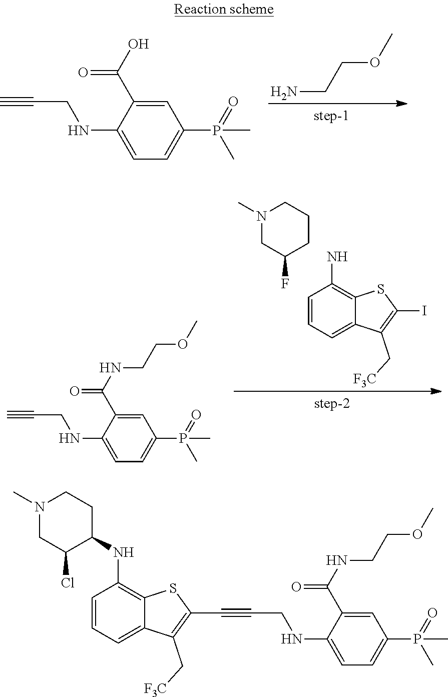

### Experiment Details

### Step 1. 5-(dimethylphosphoryl)-N-(2-methoxyethyl)-2-(prop-2-yn-1-ylamino)benzamide

Into a 8 mL vial was placed 5-(dimethylphosphoryl)-2-(prop-2-yn-1-ylamino)benzoic acid (0.10 g, 0.41 mmol), 2-methoxyethan-1-amine (0.05 g, 0.65 mmol), HATU (0.20 g, 0.53 mmol), DIEA (0.21 g, 1.62 mmol), DMF (1 mL). The reaction was stirred at R.T for 2 h under nitrogen. The residue purified by flash-C18 eluted with ACN/water (v/v=3/7) to provide the desired product. This resulted in 0.079 g (61.89% yield) of 5-(dimethylphosphoryl)-N-(2-methoxyethyl)-2-(prop-2-yn-1-ylamino)benzamide as brown solid. LCMS: m/z=309 [M+1]⁺.

### Step 2. 2-((3-(7-(((3S,4R)-3-chloro-1-methylpiperidin-4-yl)amino)-3-(2,2,2-trifluoroethyl)benzo[b]thiophen-2-yl) prop-2-yn-1-yl)amino)-5-(dimethylphosphoryl)-N-(2-methoxyethyl)benzamide (219)

Into a 20 mL vial was placed (3S,4R)-3-fluoro-N-(2-iodo-3-(2,2,2-trifluoroethyl)benzo[b]thiophen-7-yl)-1-meth ylpiperidin-4-amine (0.10 g, 0.21 mmol), 5-(dimethylphosphoryl)-N-(2-methoxyethyl)-2-(prop-2-yn-1-ylamino)benzamide (0.08 g, 0.26 mmol), bis(triphenylphosphine)palladium(II) chloride (0.03 g, 39.66 μmol), CuI (0.01 g, 36.75 μmol), DIEA (0.11 g, 0.83 mmol), Methyl sulfoxide (5 mL). The reaction was stirred at 60° C. for 2 h under nitrogen. The reaction was quenched with water (30 mL), extracted with EA (2×20 mL). The organic layers were combined, washed with brine (2×50 mL), dried over anhydrous sodium sulfate, filtered and concentrated under vacuum. The mixture was purified by preparative HPLC (Mobile Phase A: water (10 mmol/L NH₄OH), Mobile Phase B: ACN; Flow rate: 70 mL/min; Gradient: 35-68-68% B (2-30-32 min); 264 nm; RT: 27.57-29.25 min) to provide the desired product. This resulted in 0.0595 g (43.49% yield) of 2-((3-(7-(((3S,4R)-3-chloro-1-methylpiperidin-4-yl)amino)-3-(2,2,2-trifluoroethyl)benzo[b]thiophen-2-yl)prop-2-yn-1-yl)amino)-5-(dimethylphosphoryl)-N-(2-methoxyethyl)benzamide (219) as white solid. LCMS: m/z=669 [M+1]⁺.

¹H NMR (600 MHz, DMSO-d₆) δ 8.64 (t, J=5.6 Hz, 1H), 8.44 (t, J=6.1 Hz, 1H), 7.90 (d, J=11.8 Hz, 1H), 7.72-7.63 (m, 1H), 7.28 (t, J=7.8 Hz, 1H), 7.23 (d, J=8.0 Hz, 1H), 7.05-6.98 (m, 1H), 6.79 (d, J=7.7 Hz, 1H), 5.17 (d, J=8.5 Hz, 1H), 4.80 (d, J=49.4 Hz, 1H), 4.48 (d, J=6.0 Hz, 2H), 3.90-3.80 (m, 2H), 3.66 (d, J=28.8 Hz, 1H), 3.47 (t, J=5.8 Hz, 2H), 3.43 (t, J=5.6 Hz, 2H), 3.28 (s, 3H), 3.08-3.00 (m, 1H), 2.80 (d, J=11.0 Hz, 1H), 2.29-2.20 (m, 1H), 2.19 (s, 3H), 2.12-2.05 (m, 1H), 2.00-1.89 (m, 1H), 1.76-1.68 (m, 1H), 1.63 (s, 3H), 1.61 (s, 3H).

### Example 220

### (7-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)benzo[d]thiazol-4-yl)dimethylphosphine oxide (220)

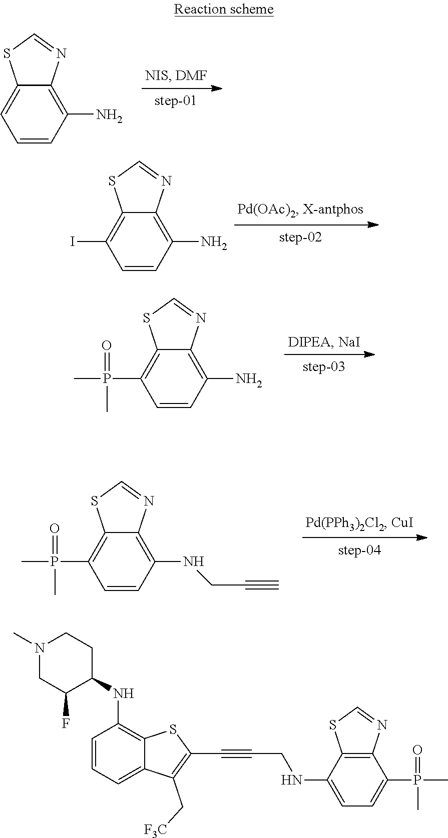

### Experimental Details

### Step 1. 7-iodobenzo[d]thiazol-4-amine

Into a 25-mL three necked bottle purged and maintained with an inert atmosphere of nitrogen, was placed benzo[d]thiazol-4-amine (1.000 g, 6.66 mmol), N,N-dimethylformamide (10 mL). The reaction mixture was cooled to −40° C. Then N-iodosuccinimide (1.302 g, 5.79 mmol) was added and stirred at the room temperature for 16 h. LCMS showed that the reaction was completed. The resulting solution was quenched with sodium bisulfite aqueous solution (5 mL) and extracted with EA (2×10 mL). The organic layer was combined and washed with brine (10 mL), dried over anhydrous Na₂SO₄, the residue was concentrated under vacuum. Slurred with H₂O (10 mL) at the room temperature for 1 h. This resulted in 1.757 g (95.58% yield) of 7-iodobenzo[d]thiazol-4-amine as yellow solid. LCMS: m/z=277 [M+1]⁺.

### Step 2. (4-aminobenzo[d]thiazol-7-yl)dimethylphosphine oxide

Into a 25-mL three necked bottle purged and maintained with an inert atmosphere of nitrogen, was placed J7-iodobenzo[d]thiazol-4-amine (1.000 g, 3.62 mmol), palladium (II) acetate (0.198 g, 881.93 μmol), dimethylbisdiphenylphosphinoxanthene (0.382 g, 660.19 μmol), N,N-diisopropylethylamine (1.086 g, 8.40 mmol), N,N-dimethylformamide (10 mL). The reaction mixture was heated to 80° C. Then methylphosphinoylmethane (0.879 g, 11.26 mmol) was added and stirred at the same temperature for 1.0 h. LCMS showed that the reaction was completed. The residue was filtered and purified by C₁₈ chromatography column eluted with ACN/H₂O (0.05% NH₄HCO₃) (v/v=1/5). This resulted in 0.83 g (101.29% yield) of (4-aminobenzo[d]thiazol-7-yl)dimethylphosphine oxide as yellow solid. LCMS: m/z=227 [M+1]⁺.

### Step 3. dimethyl(4-(prop-2-yn-1-ylamino)benzo[d]thiazol-7-yl)phosphine oxide

Into a 25-mL three necked bottle purged and maintained with an inert atmosphere of nitrogen, was placed (4-aminobenzo[d]thiazol-7-yl)dimethylphosphine oxide (0.209 g, 923.82 μmol), 3-bromopropyne (0.171 g, 1437.46 μmol), N,N-diisopropylethylamine (0.357 mg, 2762.30 μmol), sodium iodide (0.132 mg, 0.946 μmol), N,N-dimethylformamide (10 mL). The reaction mixture was stirred at 70° C. for 46 h. The resulting solution was concentrated and purified by C₁₈ chromatography column eluted with ACN/H₂O (v/v=1/5). This resulted in 0.106 g (43.42% yield) of dimethyl(4-(prop-2-yn-1-ylamino)benzo[d]thiazol-7-yl)phosphine oxide as yellow solid. LCMS: m/z=328[M+1]⁺.

### Step 4. (7-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)benzo[d]thiazol-4-yl)dimethylphosphine oxide (220)

Into a 8-mL three necked bottle purged and maintained with an inert atmosphere of nitrogen, was placed cuprous iodide (0.005 g, 26.25 μmol), triethylamine (0.063 g, 622.59 μmol), (3S,4R)-3-fluoro-N-(2-iodo-3-(2,2,2-trifluoroethyl)benzo[b]thiophen-7-yl)-1-methylpiperidin-4-amine (0.080 g, 169.39 μmol), methyl(4-(prop-2-yn-1-ylamino)benzo[d]thiazol-7-yl)phosphine oxide (0.052 g, 196.76 μmol), methyl sulfoxide (2 mL). The reaction mixture was stirred at 50 refor 18 h. LCMS showed that the reaction was completed. The resulting solution was quenched with water (10 mL) and extracted with EA (2×10 mL). The organic layer was combined and washed with brine (10 m L), dried over anhydrous Na₂SO₄, the residue was concentrated under vacuum. The mixture was purified by preparative HPLC (Mobile Phase A: water (10 mmoL/L ammonium hydroxide), Mobile Phase B: ACN; Flow rate: 40 mL/min; Gradient: 20-55-55% B (2-30-32 min; 260 nm; RT: 28.59-30.06 min). This resulted in 0.013 g (12.61% yield) of (7-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)benzo[d]thiazol-4-yl)dimethylphosphine oxide (220) as white solid. LCMS: m/z=609 [M+1]⁺.

¹H NMR (400 MHz, DMSO) δ 9.26 (s, 1H), 7.65-7.58 (m, 1H), 7.30-7.22 (m, 2H), 7.20 (d, J=7.7 Hz, 1H), 6.92 (d, J=8.4 Hz, 1H), 6.78 (d, J=7.4 Hz, 1H), 5.12 (d, J=8.3 Hz, 1H), 4.88-4.71 (m, 1H), 4.53 (d, J=5.9 Hz, 2H), 3.84-3.74 (m, 2H), 3.70-3.56 (m, 2H), 3.03 (s, 1H), 2.79 (d, J=11.0 Hz, 1H), 2.27 (d, J=13.6 Hz, 1H), 2.18 (s, 3H), 2.08 (t, J=11.8 Hz, 1H), 1.99-1.90 (m, 1H), 1.70 (s, 3H), 1.67 (s, 3H).

### Example 221

### (2-fluoro-4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-(methylthio)phenyl)dimethylphosphine oxide (221)

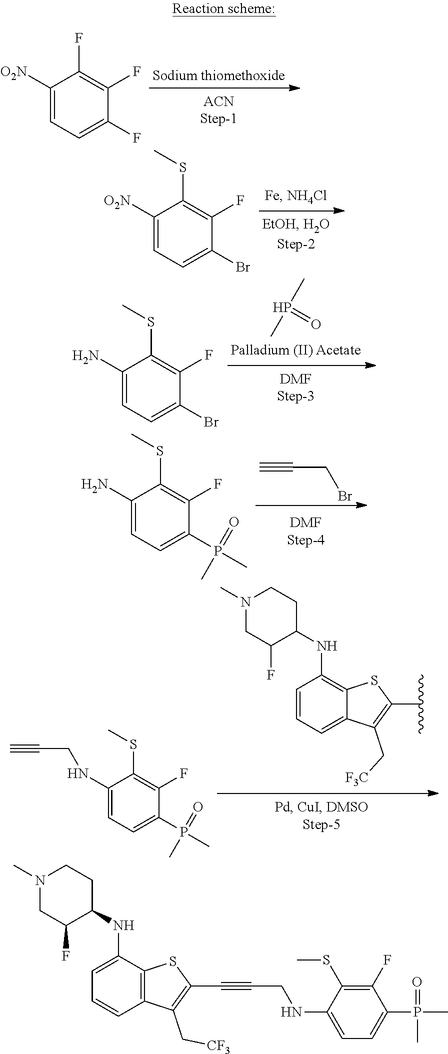

### Experiment Details

### Step 1 (3-bromo-2-fluoro-6-nitrophenyl)(methyl)sulfane

Into a 100-mL three necked bottle purged and maintained with an inert atmosphere of nitrogen, was placed 1-bromo-2,3-difluoro-4-nitrobenzene (1.534 g, 6.44 mmol) and ACN (15 mL). The reaction mixture was stirred at 0° C. Then Sodium thiomethoxide (0.476 g, 6.79 mmol) was added. The mixture was stirred at 20° C. for 1.5 h. The resulting solution was added to water (50 mL). The resulting solution was extracted with EA (3×80 mL). The organic layers combined and washed with brine (50 mL), dried over anhydrous Na₂SO₄, the residue was concentrated under vacuum. The crude product. This resulted in 1.694 g (98.76% yield) of (3-bromo-2-fluoro-6-nitrophenyl)(methyl)sulfane as yellow solid. LCMS: m/z=266 [M+1]⁺

### Step 2 4-bromo-3-fluoro-2-(methylthio)aniline

Into a 100 ml 3-necked flask was placed (3-bromo-2-fluoro-6-nitrophenyl)(methyl)sulfane (3.350 g, 59.98 m mol), Iron (3.350 g, 59.98 mmol), NH₄Cl (3.469 g, 64.85 mmol), EtOH (15 mL), water (3 mL). The reaction was stirred at 95 rfor 2 h. The reaction mixture was filtered through celite pad and celite pad was washed with methanol (2×30 mL). The filterate was then concentrated under reduced pressure to afford the product. The crude product was purified with silica gel column eluted with EA/hexane (v/v=1/5). This resulted in 1.300 g (87.72% yield) of 4-bromo-3-fluoro-2-(methylthio)aniline as yellow solid. LCMS: m/z=236 [M+1]⁺.

### Step 3 (4-amino-2-fluoro-3-(methylthio)phenyl)dimethylphosphine oxide

Into a 25 ml 3-necked flask was placed 4-bromo-3-fluoro-2-(methylthio)aniline (0.601 g, 2.54 mmol), Palladium (II) Acetate (0.119 g, 530.04 umol), Xantphos (0.287 g, 496.01 umol), DIEA (1.002 g, 7.75 mmol), DMF (6 mL) The reaction was stirred under nitrogen atmosphere at 130° C. for 0.5 h. Then dimethylphosphine oxide (0.6 mL) was added at 130° C. The reaction was stirred under nitrogen atmosphere at 130° C. for 1 h The reaction mixture was purified by C18 column eluted with ACN/H₂O (v/v=1/4), filtered and concentrated under vacuum. This resulted in 1.823 g, (112.84% yield) of (4-amino-2-fluoro-3-(methylthio)phenyl)dimethylphosphine oxide as brown oil. LCMS: m/z=234 [M+1]⁺.

### Step 4 (2-fluoro-3-(methylthio)-4-(prop-2-yn-1-ylamino)phenyl)dimethylphosphine oxide

Into a 25 ml 3-necked flask was placed (4-amino-2-fluoro-3-(methylthio)phenyl)dimethylphosphine oxide (0.311 g, 1.33 mmol), 3-bromoprop-1-yne (0.847 g, 7.12 mmol), DIEA (0.204 g, 1.57 mmol), KI (0.280 g, 1.68 mmol), DMF (4 mL). The reaction mixture was stirred under nitrogen at 60° C. for 16 h. The reaction mixture was filtered through celite pad and celite pad was washed with ACN (3×20 mL). The reaction mixture was purified by C18 column eluted with ACN/H₂O (v/v=1/2), filtered and concentrated under vacuum. This resulted in 0.064 g, (17.69% yield) of (2-fluoro-3-(methylthio)-4-(prop-2-yn-1-ylamino)phenyl)dimethylphosphine oxide as brown oil. LCMS: m/z=272 [M+1]⁺.

### Step 5 (2-fluoro-4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-(methylthio)phenyl)dimethylphosphine oxide (221)

Into a 10-mL round-bottom flask was placed (3S,4R)-3-fluoro-N-(2-iodo-3-(2,2,2-trifluoroethyl)benzo[b]thiophen-7-yl)-1-methylpiperidin-4-amine (0.073 g, 154.56 umol), (2-fluoro-3-(methylthio)-4-(prop-2-yn-1-ylamino)phenyl)dimethylphosphine oxide (0.064 g, 235.90 umol), Pd(PPh₃)₂Cl₂ (0.028 g, 39.66 μmol), CuI (0.014 g, 73.51 umol), DIEA (0.101 g, 781.47 umol), methyl sulfoxide (2 mL). The reaction was stirred under nitrogen atmosphere at RT for 16 h. The reaction mixture was filtered through celite pad and celite pad was washed with ACN (3×20 mL). The filterate was then concentrated under reduced pressure to afford the crude product The crude product was purified by preparative HPLC (Mobile Phase A: water (0.1% ammonium hydroxide), Mobile Phase B: MeOH; Flow rate: 70 mL/min; Gradient: 35-70-83% B (2-30-43 min); 265 nm; RT: 27.40-29.39) to provide the desired product. This resulted in 0.011 g (11.55% yield) of (2-fluoro-4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-(methylthio)phenyl)dimethylphosphine oxide (221) as white solid. LCMS: m/z=616 [M+1]⁺.

¹H NMR (400 MHz, DMSO-d₆) δ 7.56-7.53 (M, 1H), 7.34-7.15 (m, 2H), 6.91-6.73 (m, 3H), 5.20 (d, J=8.5 Hz, 1H), 4.80 (d, J=49.4 Hz, 1H), 4.46 (d, J=6.2 Hz, 2H), 3.80-3.77 (M, 2H), 3.75-3.61 (M, 1H), 3.04 (t, J=10.7 Hz, 1H), 2.81 (d, J=10.3 Hz, 1H), 2.24 (s, 3H), 2.19 (s, 3H), 2.10 (t, J=11.7 Hz, 1H), 2.04-1.91 (m, 2H), 1.72 (d, J=10.2 Hz, 1H), 1.63 (d, J=13.5 Hz, 6H).

### Example 222

### 2-((3S,4R)-4-((2-(3-((4-(dimethylphosphoryl)-2-methoxyphenyl)amino)prop-1-yn-1-yl)-3-(2,2,2-trifluoroethyl) benzo[b]thiophen-7-yl)amino)-3-fluoropiperidin-1-yl)-N-methylacetamide (222)

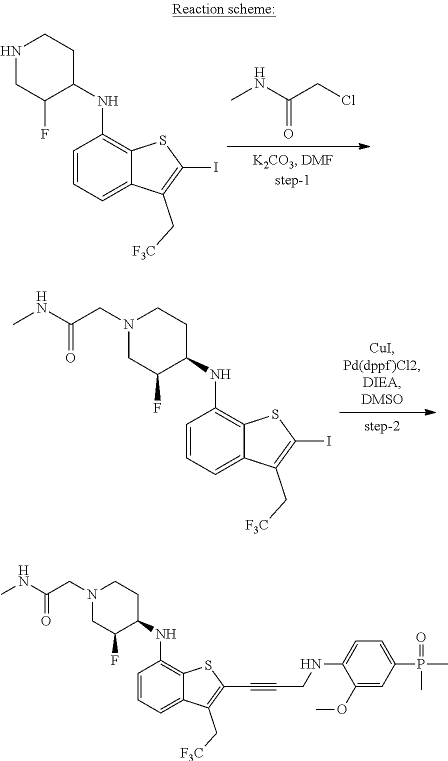

### Experiment Details

### Step 1. 2-((3S,4R)-3-fluoro-4-((2-iodo-3-(2,2,2-trifluoroethyl)benzo[b]thiophen-7-yl)amino)piperidin-1-yl)-N-methylacetamide

Into a 8-mL sealed tube purged and maintained with an inert atmosphere of nitrogen, was placed (3S,4R)-3-fluoro-N-(2-iodo-3-(2,2,2-trifluoroethyl)benzo[b]thiophen-7-yl)piperidin-4-amine (0.104 g, 0.23 mmol), 2-chloro-N-methylacetamide (0.0.35 g, 0.33), K₂CO₃ (0.067 g, 0.48 mmol), DMF (1 mL). The reaction mixture was stirred at 80° C. for 1 h. The mixture was purified with C18 column eluted with ACN/water (v/v=1/1) to afford 2-((3S,4R)-3-fluoro-4-((2-iodo-3-(2,2,2-trifluoroethyl)benzo[b]thiophen-7-yl)amino)piperidin-1-yl)-N-methylacetamide (0.073 g, 60.77% yield) as little solid. LCMS: m/z=530 [M+1]⁺

### Step 2. 2-((3S,4R)-4-((2-(3-((4-(dimethylphosphoryl)-2-methoxyphenyl)amino)prop-1-yn-1-yl)-3-(2,2,2-trifluoroethyl)benzo[b]thiophen-7-yl)amino)-3-fluoropiperidin-1-yl)-N-methylacetamide (222)

Into a 8-mL sealed tube purged and maintained with an inert atmosphere of nitrogen, was placed 2-((3S,4R)-3-fluoro-4-((2-iodo-3-(2,2,2-trifluoroethyl)benzo[b]thiophen-7-yl)amino)piperidin-1-yl)-N-methylacetamide (0.050 g, 0.09 mmol), (3-methoxy-4-(prop-2-yn-1-ylamino)phenyl)dimethylphosphine oxide (0.040 g, 0.17 mmol), Pd(dppf)Cl₂ (0.011 g, 0.03 mmol), CuI (0.023 g, 0.12 mmol), DIEA (0.058 g, 0.45 mmol), DMSO (1 mL). The reaction mixture was stirred at 50° C. for 1 h. The mixture was purified by preparative HPLC (Mobile Phase A: water (10 mmoL/L ammonium hydroxide), Mobile Phase B: MeCN; Flow rate: 70 mL/min; Gradient: 35-70-70% B (2-25-26 min)₂₆₆ nm; RT: 23.49-24.77 min) to afford 2-((3S,4R)-4-((2-(3-((4-(dimethylphosphoryl)-2-methoxyphenyl)amino)prop-1-yn-1-yl)-3-(2,2,2-trifluoroethyl)benzo[b]thiophen-7-yl)amino)-3-fluoropiperidin-1-yl)-N-methylacetamide (222) (0.0311 g, 51.47% yield) as white solid. LCMS: m/z=639 [M+1]⁺

¹H NMR (400 MHz, DMSO) δ 7.30-7.18 (m, 3H), 7.14 (d, J=11.9 Hz, 1H), 6.90-6.80 (m, 1H), 6.78 (d, J=7.7 Hz, 1H), 6.10-5.99 (m, 1H), 5.16 (d, J=8.5 Hz, 1H), 4.79 (d, J=49.5 Hz, 1H), 4.34 (d, J=6.4 Hz, 2H), 3.85 (s, 3H), 3.83-3.72 (m, 2H), 3.64 (d, J=28.0 Hz, 1H), 3.06-2.99 (m, 1H), 2.79 (d, J=12.2 Hz, 1H), 2.30-2.13 (m, 1H), 2.08 (t, J=10.9 Hz, 1H), 2.00-1.87 (m, 1H), 1.79-1.66 (m, 1H), 1.59 (s, 3H), 1.56 (s, 3H).

### Example 223

### 2-((3-(7-(((3S,4R)-3-chloro-1-methylpiperidin-4-yl)amino)-3-(2,2,2-trifluoroethyl)benzo[b]thiophen-2-yl)prop-2-yn-1-yl)amino)-5-(dimethylphosphoryl)-N-(2-methoxyethyl)benzamide (223)

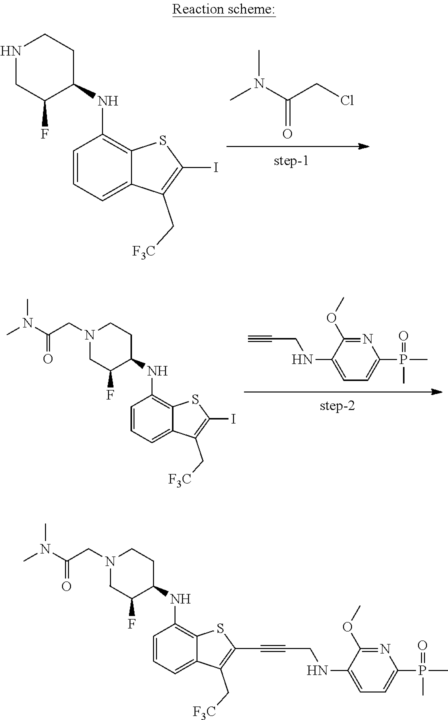

### Experiment Details

### Step 1. 2-((3S,4R)-3-fluoro-4-((2-iodo-3-(2,2,2-trifluoroethyl)benzo[b]thiophen-7-yl)amino)piperidin-1-yl)-N,N-dimethylacetamide

Into a 8 mL vial was placed (3S,4R)-3-fluoro-N-(2-iodo-3-(2,2,2-trifluoroethyl)benzo[b]thiophen-7-yl)piperidin-4-amine (0.10 g, 0.22 mol), 2-chloro-N,N-dimethylacetamide (0.07 g, 0.57 mmol), K₂CO₃ (0.12 g, 0.85 m mol), DMF (2 mL). The reaction was stirred at 80° C. for 1 h. The residue purified by flash-C18 eluted with ACN/water (v/v=2/3) to provide the desired product. This resulted in 0.095 g (79.33% yield) of 2-((3S,4R)-3-fluoro-4-((2-iodo-3-(2,2,2-trifluoroethyl)benzo[b]thiophen-7-yl)amino)piperidin-1-yl)-N,N-dimethylacetamide as yellow solid. LCMS: m/z=544 [M+1]⁺.

### Step 2. 2-((3-(7-(((3S,4R)-3-chloro-1-methylpiperidin-4-yl)amino)-3-(2,2,2-trifluoroethyl)benzo[b]thiophen-2-yl) prop-2-yn-1-yl)amino)-5-(dimethylphosphoryl)-N-(2-methoxyethyl)benzamide (223)

Into a 20 mL vial was placed 2-((3S,4R)-3-fluoro-4-((2-iodo-3-(2,2,2-trifluoroethyl)benzo[b]thiophen-7-yl)amino)piperidin-1-yl)-N,N-dimethylacetamide (0.09 g, 0.17 mmol), (6-methoxy-5-(prop-2-yn-1-ylamino)pyridin-2-yl)dimethylphosphine oxide (0.08 g, 0.32 mmol), bis(triphenylphosphine)palladium(II) chloride (0.03 g, 46.75 μmol), CuI (0.01 g, 42.01 μmol), DIEA (0.32 g, 2.45 mmol), Methyl sulfoxide (5 mL). The reaction was stirred at 60° C. for 1 h under nitrogen. The reaction was quenched with water (30 mL), extracted with EA (2×20 mL). The organic layers were combined, washed with brine (2×50 mL), dried over anhydrous sodium sulfate, filtered and concentrated under vacuum. The mixture was purified by preparative HPLC (Mobile Phase A: water (10 mmol/L ammonium hydroxide), Mobile Phase B: ACN; Flow rate: 70 mL/min; Gradient: 35-70-72% B (2-25-27 min); 266 nm; RT: 24.73-26.01 min) to provide the desired product. This resulted in 0.0252 g (22.28% yield) of 2-((3-(7-(((3S,4R)-3-chloro-1-methylpiperidin-4-yl)amino)-3-(2,2,2-trifluoroethyl)benzo[b]thiophen-2-yl)prop-2-yn-1-yl)amino)-5-(dimethylphosphoryl)-N-(2-methoxyethyl)benzamide (223) as white solid. LCMS: m/z=654 [M+1]⁺.

¹H NMR (600 MHz, DMSO) δ 7.44-7.35 (m, 1H), 7.32-7.26 (m, 1H), 7.23 (d, J=7.8 Hz, 1H), 7.10-7.02 (m, 1H), 6.80 (d, J=7.6 Hz, 1H), 6.40 (t, J=6.3 Hz, 1H), 5.27-5.12 (m, 1H), 4.94-4.73 (m, 1H), 4.35 (d, J=6.2 Hz, 2H), 3.94 (s, 3H), 3.87-3.76 (m, 2H), 3.76-3.61 (m, 1H), 3.29-3.0 (m, 3H), 3.01 (s, 3H), 2.84 (s, 3H), 2.65-2.54 (m, 1H), 2.39 (s, 1H), 2.15-1.88 (m, 2H), 1.84-1.67 (m, 1H), 1.59 (s, 3H), 1.57 (s, 3H).

### Example 224

### 6-(dimethylphosphoryl)-3-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)picolinonitrile (224)

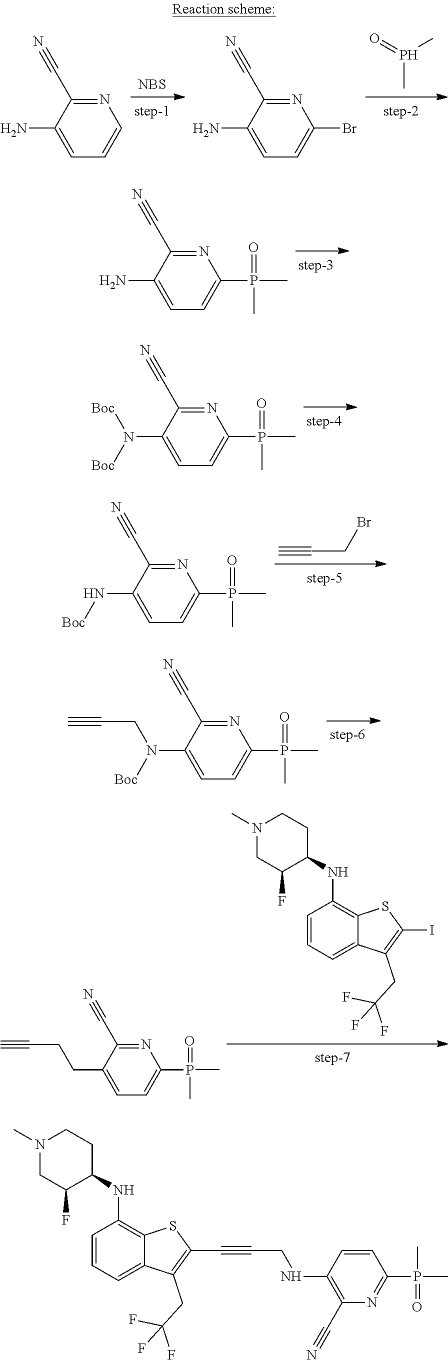

### Experiment Details

### Step 1. 3-amino-6-bromopicolinonitrile

Into a 40 mL sealed tube was placed 3-aminonicotinonitrile (0.51 g, 4.29 mmol), NBS (0.80 g, 4.51 mmol), MeCN (10 mL). The reaction was stirred at 20° C. for 2 h. The reaction was purified by silica gel column eluted with EA/hexane (v/v=3/7). This resulted in 0.521 g (61.33% yield) of 3-amino-6-bromopicolinonitrile as brown solid. LCMS: m/z=198 [M+1]⁺.

### Step 2. 3-amino-6-(dimethylphosphoryl)picolinonitrile

Into a 50 mL three round bottom flask was placed 3-amino-6-bromopicolinonitrile (0.50 g, 2.51 mmol), Palladium (II) acetate (0.12 g, 0.53 mmol), Dimethylbisdiphenylphosphinoxanthene (0.33 g, 0.56 mmol), DIEA (1.04 g, 8.07 mmol), DMF (10 mL). The reaction was heat to 130° C. under nitrogen. Dimethylphosphine oxide (0.59 g, 7.50 mmol) was added to the reaction slowly. The mixture was stirred at 130° C. for 1 h. The residue purified by flash-C18 eluted with ACN/water (v/v=1/4) to provide the desired product. This resulted in 0.608 g (crude) of 3-amino-6-(dimethylphosphoryl)picolinonitrile as off-white solid. LCMS: m/z=196 [M+1]⁺.

### Step 3. tert-butyl N-tert-butoxycarbonyl-N-(2-cyano-6-dimethylphosphoryl-3-pyridyl)carbamate

Into a 100 mL round-bottom flask was placed (6-amino-5-methoxypyridin-3-yl)dimethylphosphine oxide (0.55 g, 2.80 mmol), Di-tert-butyl dicarbonate (3.06 g, 14.01 mmol), N-(4-pyridyl)dimethylamine (0.13 g, 1.08 mmol), 1,4-Dioxane (10 mL). The reaction was stirred at 110° C. for 1 h. The mixture was concentrated un der vacuum. This resulted in 0.790 g (71.42% yield) of tert-butyl N-tert-butoxycarbonyl-N-(2-cyano-6-dimethylphosphoryl-3-pyridyl)carbamate as brown oil. LCMS: m/z=396 [M+1]⁺.

### Step 4. tert-butyl (2-cyano-6-(dimethylphosphoryl)pyridin-3-yl)carbamate

Into a 50 mL round-bottom flask was placed tert-butyl N-tert-butoxycarbonyl-N-(2-cyano-6-dimethylphosphoryl-3-pyridyl)carbamate (0.78 g, 1.98 mmol), K₂CO₃ (1.00 g, 7.32 mmol), MeOH (10 mL). The reaction was stirred at 70° C. for 1 h. The mixture was concentrated under vacuum. The reaction was quenched by water (50 mL), extracted with EA (2×50 mL). The organic layers were combined, washed with brine (2×100 mL), dried over anhydrous sodium sulfate, filtered and concentrated under vacuum. This resulted in 0.346 g (59.17% yield) of tert-butyl (2-cyano-6-(dimethylphosphoryl)pyridin-3-yl)carbamate as brown solid. LCMS: m/z=296 [M+1]⁺.

### Step 5. tert-butyl (2-cyano-6-(dimethylphosphoryl)pyridin-3-yl)(prop-2-yn-1-yl)carbamate

Into a 100 mL round-bottom flask was placed tert-butyl (2-cyano-6-(dimethylphosphoryl)pyridin-3-yl)carbamate (0.34 g, 1.15 mmol), THF (20 mL). The reaction was stirred at 0° C. NaH (0.21 g, 8.75 mmol) was added to the reaction slowly at 0° C. and stirred for 0.5 h. 3-bromoprop-1-yne (0.799 g, 6.72 mmol) was added to the reaction and stirred at 60° C. for 2 h. The reaction was quenched by water (5 mL) and concentrated under vacuum. The residue purified by flash-C18 eluted with ACN/water (v/v=1/4) to provide the desire d product. This resulted in 0.238 g (61.83% yield) of tert-butyl (2-cyano-6-(dimethylphosphoryl)pyridin-3-yl) (prop-2-yn-1-yl)carbamate as brown oil. LCMS: m/z=334 [M+1]⁺.

### Step 6. 6-(dimethylphosphoryl)-3-(prop-2-yn-1-ylamino)picolinonitrile

Into a 50 mL round-bottom flask was placed tert-butyl (2-cyano-6-(dimethylphosphoryl)pyridin-3-yl)(prop-2-yn-1-yl)carbamate (0.24 g, 0.71 mmol), TFA (3 mL), DCM (10 mL). The reaction was stirred at R.T for 0.5 h. The reaction was concentrated under vacuum. The residue purified by flash-C18 eluted with ACN/water (v/v=1/9) to provide the desired product. This resulted in 0.101 g (60.66% yield) of 6-(dimethylphosphoryl)-3-(prop-2-yn-1-ylamino)picolinonitrile as brown solid. LCMS: m/z=234 [M+1]⁺.

### Step 7. 6-(dimethylphosphoryl)-3-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)picolinonitrile (224)

Into a 40 mL vial was placed (3S,4R)-3-fluoro-N-(2-iodo-3-(2,2,2-trifluoroethyl)benzo[b]thiophen-7-yl)-1-methylpiperidin-4-amine (0.12 g, 0.25 mmol), 6-(dimethylphosphoryl)-3-(prop-2-yn-1-ylamino)picolinonitrile (0.09 g, 0.39 mmol), bis(triphenylphosphine)palladium(II) chloride (0.06 g, 77.91 μmol), CuI (0.01 g, 52.51 μmol), DIEA (0.09 g, 0.70 mmol), Methyl sulfoxide (5 mL). The reaction was stirred at 60° C. for 3 h under nitrogen. The reaction was quenched with water (30 mL), extracted with EA (2×20 mL). The organic lay ers were combined, washed with brine (2×50 mL), dried over anhydrous sodium sulfate, filtered and concentrated under vacuum. The mixture was purified by preparative HPLC (Mobile Phase A: water (10 mmol/L NH₄OH), Mobile Phase B: ACN; Flow rate: 70 mL/min; Gradient: 35-67-67% B (2-30-33 min); 270 nm; RT: 28.86-29.72 min) to provide the desired product. This resulted in 0.0124 g (8.59% yield) of 6-(dimethylphosphoryl)-3-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)picolinonitrile (224) as white solid. LCMS: m/z=578 [M+1]⁺.

¹H NMR (600 MHz, DMSO-d₆) δ 8.01-7.95 (m, 1H), 7.65-7.61 (m, 1H), 7.54 (t, J=6.1 Hz, 1H), 7. 28 (t, J=7.8 Hz, 1H), 7.23 (d, J=8.0 Hz, 1H), 6.80 (d, J=7.7 Hz, 1H), 5.19 (d, J=8.5 Hz, 1H), 4.80 (d, J=49.5 Hz, 1H), 4.48 (d, J=5.9 Hz, 2H), 3.89-3.80 (m, 2H), 3.66 (d, J=29.0 Hz, 1H), 3.04 (t, J=11.3 Hz, 1H), 2.80 (d, J=11.2 Hz, 1H), 2.26 (d, J=13.0 Hz, 1H), 2.19 (s, 3H), 2.12-2.0 (m, 1H), 2.01-1.90 (m, 1H), 1.75-1.69 (m, 1H), 1.63 (s, 3H), 1.61 (s, 3H).

### Example 225

### (3-(difluoromethyl)-4-((3-(7-(((3R,4S)-3-fluoro-1-methylpiperidin-4-yl)amino)-3-(2,2,2-trifluoroethyl)benzol[b]thiophen-2-yl)prop-2-yn-1-yl)amino)phenyl)dimethylphosphine oxide (225)

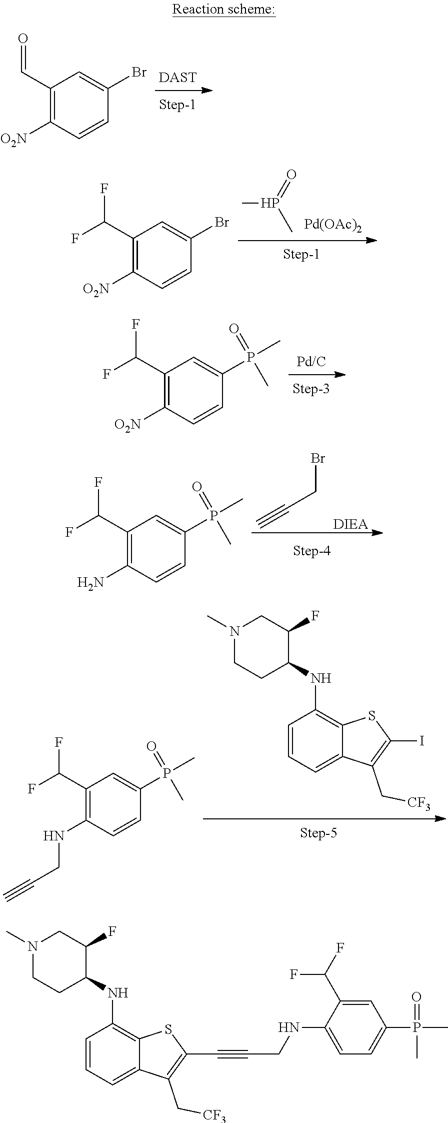

### Experiment Details

### Step 1. 4-bromo-2-(difluoromethyl)-1-nitrobenzene

Into a 250 mL flask was placed (5-bromo-2-nitrobenzaldehyde (10.12 g, 44.00 mmol), DCM (100 mL) and stirred. The reaction was cooled to −20° C. then DAST (14.01 g, 86.92 mmol) was added in portion. The reaction was warmed to room temperature and stirred for 1.5 h. The reaction was quenched with water (150 mL), extracted with EA (100 mL×3). The combined organic layer was washed with water (100 mL) and brine (100 mL) successively, separated, then concentrated under vacuum to afford 4-bromo-2-(difluoromethyl)-1-nitrobenzene (0.027 g, 43.64 μmol, 42.41% yield) as off-white solid. LCMS: m/z=252 [M+1]⁺.

### Step 2. (3-(difluoromethyl)-4-nitrophenyl)dimethylphosphine oxide

Into a 500 mL flask purged and maintained with N₂ was placed 4-bromo-2-(difluoromethyl)-1-nitrobenzene (4.532 g, 17.98 mmol), Palladium (II) acetate, (0.459 g, 2.04 mmol), XantPhos (2.361 g, 4.08 mmol), DIE A (4.651 g, 35.99 mmol), DMF (40 mL). The reaction was heated to 120° C. and dimethylphosphine oxide (2.399 g, 30.74 mmol) in DMF (2 mL) was add to the solution while stirring. The solution was stirred for 2 h. The solution was cooled to room temperature, and poured to water 250 mL. The solid was filtered, and the filter cake was washed with water 50 mL. The filtration was concentrate under vacuum and purified with C₁₈ column, eluted with ACN/water (v/v=3/10) to afford (3-(difluoromethyl)-4-nitrophenyl)dimeth ylphosphine oxide as off-white solid. LCMS: m/z=250[M+1]⁺

### Step 3. (4-amino-3-(difluoromethyl)phenyl)dimethylphosphine oxide

Into a 50 mL flask was placed (3-(difluoromethyl)-4-nitrophenyl)dimethylphosphine oxide (3.215 g, 12.90 m mol), Pd/C (1.502 g, 14.11 mmol), EtOH (15 mL). The reaction was stirred overnight at Hydrogen atmosphere, room temperature. The reaction was filtered, the filter cake was washed with EtOH (5 mL×2). The filtrate was concentrated to afford (4-amino-3-(difluoromethyl)phenyl)dimethylphosphine oxide (2.571 g, 11.73 mmol, 90.91% yield) as off-white solid. LCMS: m/z=220[M+1]⁺

### Step 4. (3-(difluoromethyl)-4-(prop-2-yn-1-ylamino)phenyl)dimethylphosphine oxide

Into a 100 mL flask was placed (4-amino-3-(difluoromethyl)phenyl)dimethylphosphine oxide (2.370 g, 10.81 mmol), 3-bromoprop-1-yne (1.665 g, 14.00 mmol 1), K₂CO₃ (2.447 g, 17.71 mmol), DMF (15 mL). The reaction was stirred overnight at 50° C. The reaction was purified with C₁₈ column, eluted with ACN/water (v/v=1/1) to afford 3-(difluoromethyl)-4-(prop-2-yn-1-ylamino)phenyl)dimethylphosphine oxide (0.576 g, 2.24 mmol, 20.71% yield) as white solid. LCMS: m/z=258[M−1]⁺

### Step 5. (3-(difluoromethyl)-4-((3-(7-(((3R,4S)-3-fluoro-1-methylpiperidin-4-yl)amino)-3-(2,2,2-trifluoroethyl) benzo[b]thiophen-2-yl)prop-2-yn-1-yl)amino)phenyl)dimethylphosphine oxide (225)

Into a 4 mL vial purged and maintained with nitrogen atmosphere was placed (3R,4S)-3-fluoro-N-(2-iodo-3-(2,2,2-trifluoroethyl)benzo[b]thiophen-7-yl)-1-methylpiperidin-4-amine (0.101 g, 213.8557 μmol), Pd(PPh₃)₂Cl₂ (0.014 g, 19.83 μmol), CuI (0.004 g, 21.00 μmol), TEA (0.048 g, 474.36 μmol), DMF (1 mL). The reaction was heated to 8036 μmol), benzo[b]thiophen-7-yl)-1-methylpiperidin-4-amine (thiophen-2-yl)prop-2-yn-1-yl) ami) was added. The reaction was stirred for 1 h at 80 hm The reaction was diluted with water (4 mL), extracted with EA (4 mL×2). The combined organic layers were washed with water (2 mL) and brine (2 mL) successively, separated, then concentrated with vacuum. The residue was purified with prep-HPLC (Mobile Phase A: water (formic acid), Mobile Phase B: ACN; Flow rate: 70 mL/min; Gradient: 35-69-69% B (2-30-35 min); 262 nm; RT: 29.95-31.52) to afford (3-(difluoromethyl)-4-((3-(7-(((3R,4S)-3-fluoro-1-methylpiperidin-4-yl)amino)-3-(2,2,2-trifluoroethyl)benzo[b]thiophen-2-yl)prop-2-yn-1-yl)amino)phenyl)dimethylphosphine oxide (225) (0.032 g, 53.19 μmol, 24.87% yield) as white solid. LCMS: m/z=580 [M+1]⁺.

¹H NMR (400 MHz, DMSO-d₆) δ 7.83-7.66 (m, 2H), 7.29-7.19 (m, 2H), 7.15-6.93 (m, 2H), 6.78 (d, J=7.6 Hz, 1H), 6.60 (t, J=6.0 Hz, 1H), 5.15 (d, J=8.4 Hz, 1H), 4.79 (d, J=49.7 Hz, 1H), 4.42 (d, J=5.9 Hz, 2H), 3.81 (q, J=11.0 Hz, 2H), 3.65 (d, J=28.8 Hz, 1H), 3.03 (t, J=11.6 Hz, 1H), 2.79 (d, J=11.2 Hz, 1H), 2.27 (d, J=13.1 Hz, 1H), 2.18 (s, 3H), 2.08 (t, J=11.5 Hz, 1H), 2.00-1.87 (m, 1H), 1.72 (d, J=12.4 Hz, 1H), 1.59 (d, J=13.3 Hz, 6H).

### Example 226&227 (S)-(4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)(imino)(methyl)-16-sulfanone (226) and (R)-(4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)(imino)(methyl)-16-sulfanone (227) PGP-283C

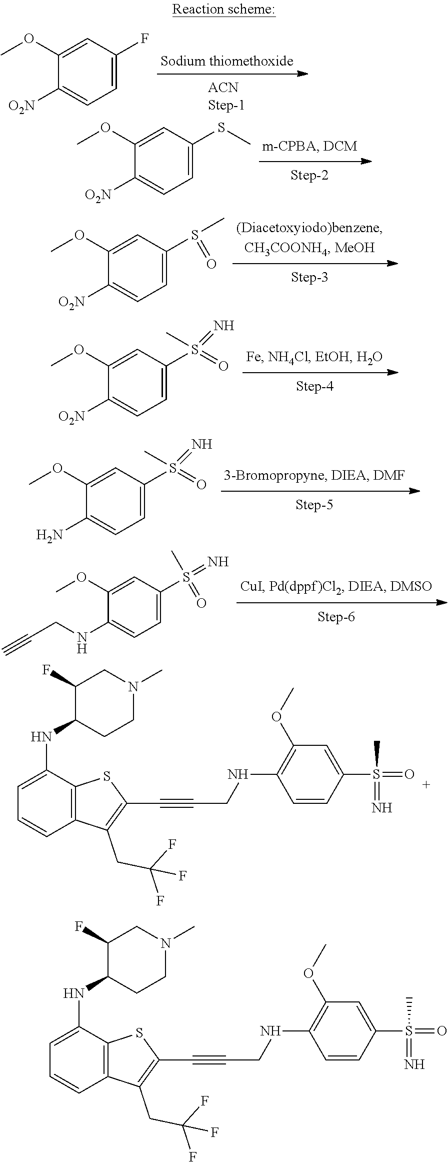

### Experiment Details

### Step 1. (3-methoxy-4-nitrophenyl)(methyl)sulfane

Into a 500-mL round-bottom flask was placed 4-fluoro-2-methoxy-1-nitrobenzene (10.136 g, 59.23 mmol), ACN (100 mL). Sodium thiomethoxide (8.266 g, 0.12 mol) was added at 0° C. The reaction mixture was stirred at room temperature for 1 h. The reaction was quenched with water (100 mL), extracted with EA (150 mL×3). The organic layers were combined, washed with brine (50 mL), dried over anhydrous sodium sulfate, filtered and concentrated under vacuum. The mixture was purified by silica gel chromatography eluted with EA/hexane (v/v=1:3) to afford (3-methoxy-4-nitrophenyl)(methyl)sulfane (6.444 g, 54.61% yield) as brown solid. LCMS: m/z=200 [M+1]⁺

### Step 2. 2-methoxy-4-(methylsulfinyl)-1-nitrobenzene

Into a 100-mL round-bottom flask was placed (3-methoxy-4-nitrophenyl)(methyl)sulfane (2.054 g, 10.31 mmol), DCM (20 mL). 3-Chloroperoxybenzoic acid (1.715 g, 9.94 mmol) was added at 0° C. The reaction mixture was stirred at room temperature for 1 h. The reaction was quenched with 1 M KHCO₃ (100 mL), extracted with EA (150 mL×3). The organic layers were combined, washed with brine (50 mL), dried over anhydrous sodium sulfate, filtered and concentrated under vacuum. The mixture was purified by silica gel chromatography eluted with EA/hexane (v/v=1:4) to afford 2-methoxy-4-(methylsulfinyl)-1-nitrobenzene (1.672 g, 75.35% yield) as little solid. LCMS: m/z=216 [M+1]⁺

### Step 3. imino(3-methoxy-4-nitrophenyl)(methyl)-16-sulfanone

Into a 100-mL round-bottom flask was placed 2-methoxy-4-(methylsulfinyl)-1-nitrobenzene (1.469 g, 6.83 m mol), Ammonium acetate (2.078 g, 26.96 mmol), (Diacetoxyiodo)benzene (8.875 g, 27.55 mol), MeOH (20 mL). The reaction mixture was stirred at room temperature for 4 h. The reaction was quenched with H₂O (100 mL), extracted with EA (250 mL×2). The organic layers were combined, washed with brine (50 m L×2), dried over anhydrous sodium sulfate, filtered and concentrated under vacuum. The mixture was purified by silica gel chromatography eluted with EA/hexane (v/v=2:1) to afford imino(3-methoxy-4-nitrophenyl) (methyl)-16-sulfanone (1.364 g, 86.80% yield) as off-white solid. LCMS: m/z=231 [M+1]⁺

### Step 4. (4-amino-3-methoxyphenyl)(imino)(methyl)-16-sulfanone

Into a 100-mL round-bottom flask was placed imino(3-methoxy-4-nitrophenyl)(methyl)-16-sulfanone (0.543 g, 2.36 mmol), Iron (0.595 g, 10.65 mmol), NH₄Cl (1.140 g, 21.31 mol), EtOH (10 mL) and H₂O (2 mL). The reaction mixture was stirred at 70° C. for 1 h. The mixture filtered and concentrated under vacuum. The crude was stirred with 20 mL (MeOH/DCM (1/20)) over night. The mixture was filtered through a Celite pad, and the filtrate was concentrated to afford (4-amino-3-methoxyphenyl)(imino)(methyl)-16-sulfanone (0.629 g, crude) as yellow oil. LCMS: m/z=201 [M+1]⁺

### Step 5. imino(3-methoxy-4-(prop-2-yn-1-ylamino)phenyl)(methyl)-16-sulfanone

Into a 20-mL sealed tube was placed (4-amino-3-methoxyphenyl)(imino)(methyl)-16-sulfanone (0.285 g, 1.42 mmol), 3-Bromopropyne (0.120 g, 1.01 mmol), DIEA (0.352 g, 2.72 mmol), DMF (4 mL). The reaction mixture was stirred at 50° C. for 12 h. The mixture purified by flash-C18 eluted with ACN/water (v/v=1/7) to provide the desired product. This resulted in 0.112 g (33.03% yield) of imino(3-methoxy-4-(prop-2-yn-1-ylamino)phenyl)(methyl)-16-sulfanone as yellow oil. LCMS: m/z=239 [M+1]⁺

### Step 6. (4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)(imino)(methyl)-16-sulfanone

Into a 8-mL sealed tube purged and maintained with an inert atmosphere of nitrogen, was placed (3S,4R)-3-fluoro-N-(2-iodo-3-(2,2,2-trifluoroethyl)benzo[b]thiophen-7-yl)-1-methylpiperidin-4-amine (0.140 g, 0.30 mmol), imino(3-methoxy-4-(prop-2-yn-1-ylamino)phenyl)(methyl)-16-sulfanone (0.061 g, 0.26 mmol), Pd(dppf)Cl₂ (0.016 g, 0.02 mmol), CuI (0.021 g, 0.11 mmol), DIEA (0.147 g, 1.13 mmol), DMSO (1 mL). The reaction mixture was stirred at 50° C. for 1 h. The mixture was purified by preparative HPLC (Mobile Phase A: water (10 mmoL/L ammonium hydroxide), Mobile Phase B: MeCN; Flow rate: 70 mL/min; Gradient: 40-75-95% B (2-32-60 min) 260 nm; RT: 31.40-33.96 min). The racemic product was separated by Prep-HPLC-Gilson with the following conditions: Column, CHIRALART Cellulose-SC column (2 cm×25 cm, 5 um); mobile phase, (HeX:DcM=1:1)(0.1% IPA.M)/EtOH (50:50); Flowing rate: 18 ml/min. This results in (0.0148 g)(S)-(4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)(imino)(methyl)-16-sulfanone (226) (the first eluting isomer, Retention Time 5.253 min). LCMS: m/z=583 [M+1]⁺

¹H NMR (400 MHz, DMSO) δ 7.47-7.39 (m, 1H), 7.30 (d, J=1.9 Hz, 1H), 7.27 (d, J=7.8 Hz, 1H), 7.21 (d, J=8.0 Hz, 1H), 6.87 (d, J=8.4 Hz, 1H), 6.78 (d, J=7.7 Hz, 1H), 6.40-6.30 (m, 1H), 5.18 (d, J=8.6 Hz, 1H), 4.89-4.71 (m, 1H), 4.38 (d, J=6.3 Hz, 2H), 3.88 (s, 3H), 3.86-3.75 (m, 2H), 3.75-3.57 (m, 2H), 3.09-3.02 (m, 1H), 2.99 (s, 3H), 2.80 (d, J=10.3 Hz, 1H), 2.37-2.24 (m, 1H), 2.19 (s, 3H), 2.16-2.04 (m, 1H), 2.00-1.88 (m, 1H), 1.80-1.66 (m, 1H).

And (0.0144 g)(R)-(4-((3-(7-(((3S,4R)-3-fluoro-1-methylpiperidin-4-yl)amino)-3-(2,2,2-trifluoroethyl)benzo[b]thiophen-2-yl)prop-2-yn-1-yl)amino)-3-methoxyphenyl)(imino)(methyl)-16-sulfanone (227) (the second eluting isomer, Retention Time 7.01 min) LCMS: m/z=583 [M+1]⁺

¹H NMR (400 MHz, DMSO) δ 7.47-7.39 (m, 1H), 7.30 (d, J=1.9 Hz, 1H), 7.27 (d, J=7.8 Hz, 1H), 7.21 (d, J=8.0 Hz, 1H), 6.87 (d, J=8.4 Hz, 1H), 6.78 (d, J=7.7 Hz, 1H), 6.40-6.30 (m, 1H), 5.18 (d, J=8.6 Hz, 1H), 4.89-4.71 (m, 1H), 4.38 (d, J=6.3 Hz, 2H), 3.88 (s, 3H), 3.86-3.75 (m, 2H), 3.75-3.57 (m, 2H), 3.09-3.02 (m, 1H), 2.99 (s, 3H), 2.80 (d, J=10.3 Hz, 1H), 2.37-2.24 (m, 1H), 2.19 (s, 3H), 2.16-2.04 (m, 1H), 2.00-1.88 (m, 1H), 1.80-1.66 (m, 1H).

### Ref-1a and Ref-1b

### N-((3S,4R)-3-fluoro-1-methylpiperidin-4-yl)-2-(3-((2-methoxy-4-(methylsulfonyl)phenyl)amino)prop-1-yn-1-yl)-1-(2,2,2-trifluoroethyl)-1H- indol-4-amine (Ref-1a) and N-((3R,4S)-3-fluoro-1-methylpiperidin-4-yl)-2-(3-((2-methoxy-4-(methylsulfonyl)phenyl)amino)prop-1-yn-1-yl)-1-(2,2,2-trifluoroethyl)-1H-indol-4-amine (Ref-1b)

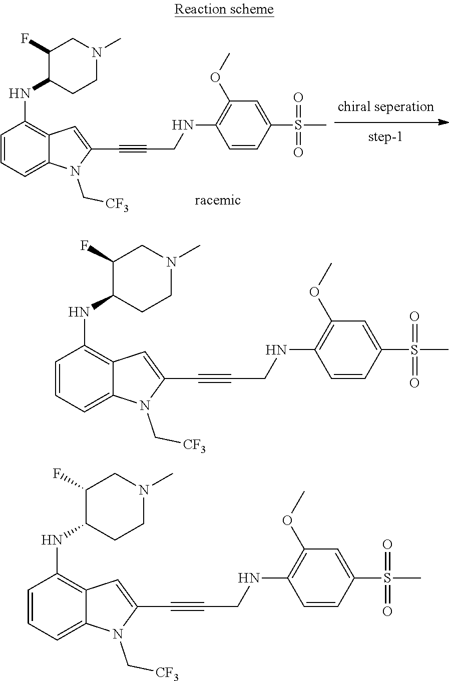

### Experimental Details

### Step 1. N-((3S,4R)-3-fluoro-t-methylpiperidin-4-yl)-2-(3-((2-methoxy-4-(methylsulfonyl)phenyl)amino)prop-1-yn-1-yl)-1-(2,2,2-trifluoroethyl)-1H-indol-4-amine (Ref-1a) and N-((3R,4S)-3-fluoro-t-methylpiperidin-4-yl)-2-(3-((2-methoxy-4-(methylsulfonyl)phenyl)amino)prop-1-yn-1-yl)-1-(2,2,2-trifluoroethyl)-1H-indol-4-amine (Ref-1b)

The racemic sample N—((Z)-3-fluoro-1-methylpiperidin-4-yl)-2-(3-((2-methoxy-4-(methylsulfonyl)phenyl)amino)prop-1-yn-1-yl)-1-(2,2,2-trifluoroethyl)-1H-indol-4-amine (1.00 g, 1.77 mmol) was separated with pre-chiral-HPLC with (Column:SB, Mobile Phase A: (HeX:DcM=3:1)(0.1% IPA.M), Mobile Phase B: EtOH; Flow rate: 20 mL/min; A:B=50:50; 220 nm; RT: 7.884 min) to provide 418.00 mg (41.67% yield) of N-((3R,4S)-3-fluoro-1-methylpiperidin-4-yl)-2-(3-((2-methoxy-4-(methylsulfonyl)phenyl)amino)prop-1-yn-1-yl)-1-(2,2,2-trifluoroethyl)-1H-indol-4-amine (Ref-1b) as white solid. LCMS: m/z=567 [M+1]⁺

¹H NMR (400 MHz, DMSO-d₆) δ 7.39 (d, J=8.3 Hz, 1H), 7.25 (s, 1H), 7.19 (s, 1H), 7.01 (t, J=7.9 Hz, 1H), 6.89 (d, J=8.3 Hz, 1H), 6.74 (d, J=8.3 Hz, 1H), 6.48 (t, J=6.0 Hz, 1H), 6.24 (d, J=7.8 Hz, 1H), 5.49 (d, J=8.6 Hz, 1H), 4.89-4.83 (m, 3H), 4.36 (d, J=6.1 Hz, 2H), 3.89 (s, 3H), 3.56 (d, J=29.6 Hz, 1H), 3.17-2.95 (m, 4H), 2.80 (d, J=10.8 Hz, 1H), 2.35-2.01 (m, 6H), 1.95-1.90 (m, 1H), 1.69-1.66 (m, 1H).

The racemic sample N-(3-fluoro-1-methylpiperidin-4-yl)-2-(3-((2-methoxy-4-(methylsulfonyl)phenyl)amino)prop-1-yn-1-yl)-1-(2,2,2-trifluoroethyl)-1H-indol-4-amine (1.00 g, 1.77 mmol) was separated with pre-chiral-HPLC with (Column:SB, Mobile Phase A: (HeX:DcM=3:1)(0.1% IPA.M), Mobile Phase B: EtOH; Flow rate: 20 mL/min; A:B=50:50; 220 nm; RT: 9.707 min) to provide 415.00 mg (41.37% yield) of N-((3S,4R)-3-fluoro-1-methylpiperidin-4-yl)-2-(3-((2-methoxy-4-(methylsulfonyl)phenyl)amino)prop-1-yn-1-yl)-1-(2,2,2-trifluoroethyl)-1H-indol-4-amine (Ref-1a) as white solid. LCMS: m/z=567 [M+1]⁺

¹H NMR (400 MHz, DMSO-d₆) δ 7.37 (t, J=17.4 Hz, 1H), 7.24 (d, J=13.3 Hz, 1H), 7.19 (s, 1H), 7. 01 (t, J=8.0 Hz, 1H), 6.90 (d, J=8.4 Hz, 1H), 6.74 (d, J=8.2 Hz, 1H), 6.55-6.50 (m, 1H), 6.24 (d, J=7.8 Hz, 1H), 5.52 (d, J=8.7 Hz, 1H), 4.94-6.90 (m, 3H), 4.36 (d, J=6.2 Hz, 2H), 3.89 (s, 3H), 3.65-3.60 (m, 1H), 3.19-2.97 (m, 4H), 2.84-2.80 (m, 1H), 2.32-2.04 (m, 5H), 1.95-1.90 (m, 1H), 1.69-1.65 (m, 1H).

### Pharmacological Experiments

### 1. In Vitro DNA Binding Assay.

After His-p53 Y220C (94-294) protein was placed with a compound in the present of MAb Anti His-Tb cryptate (Cisbio, Cat. NO. 61H12TLA) in a 384 well microplate (Greiner) for 15 min at 25° C., the assay plate was transferred into 27° C. incubator and incubated for 60 min. Biotinalyted p53 consensus DNA (ID: p53-SEQUENCE 2-F: 5′(biotin)-ATTAGGCATGTCTAGGCATGTCTAGG 3′ —R:5′ CCTAGACATGCCTAGACATGCCTAAT 3′) was added into the assay plate in the present of Streptaidin-d2 (Cisbio, Cat. NO. 610SADLF) cryptate (Final concentration:20 mM HEPES pH=7.4, 75 mM KCl, 1 mM MgCl₂, 0.1% (w/v) BSA, 1 mM DTT, 2.5 nM His-P53 Y220C (94-294) protein, 0.33 nM MAb Anti His-Tb cryptate, 2.5 nM Streptaidin-d2 cryptate, 10 nM Biotinalyted DNA) and incubated for another 60 min. Wells containing 2 μM reference compound Ref-ta served as high control, and wells containing same percentage of DMSO served as low control. Homogeneous time-resolved fluorescence (HTRF) signals were read on Tecan Spark multimode microplate reader. HTRF ratios for each individual well were calculated by equation: HTRF ratio=(Signal F665/Signal F620)*1000. The percent of activation of compounds treated wells were normalized between high control and low control (% Activation=(HTRF ratio_{compound treated}−HTRF ratio_{low control})/(HTRF ratio_{high control}−HTRF ratio_{low control})*100%). Then the data were analyzed either by fitting a 4-parameter logistic model or by Excel to calculate EC₅₀ values. And the EC₅₀ was the concentration at 50% Activation on the curve.

The structure of reference compound:

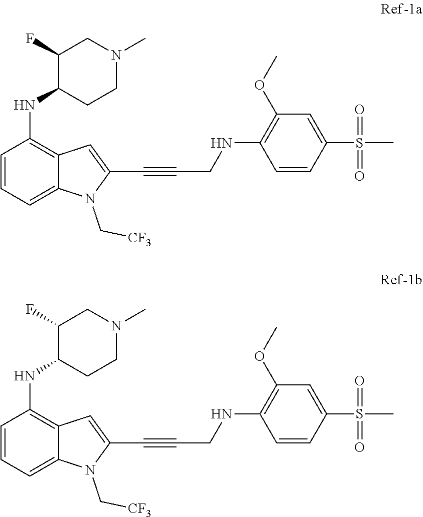 [SMILES: COc1cc(S(C)(=O)=O)ccc1NCC#Cc1cc2c(N[C@@H]3CCN(C)C[C@@H]3F)cccc2n1CC(F)(F)F ; COc1cc(S(C)(=O)=O)ccc1NCC#Cc1cc2c(N[C@H]3CCN(C)C[C@H]3F)cccc2n1CC(F)(F)F]

The p53(Y220C) EC₅₀ (μM) values of some example compounds of the present invention weir shown in the following table 1.

TABLE 1

| Comp. Ex. No. | p53(Y220C) EC₅₀ (μM) |
|---|---|
| Ref-1a | 0.048 |
| Ref-1b | 0.096 |
| 1 | 0.345 |
| 2 | 0.044 |
| 3 | 0.081 |
| 4 | 0.099 |
| 5 | 0.032 |
| 6 | 0.130 |
| 7 | 0.200 |
| 8 | 0.112 |
| 9 | 0.114 |
| 10 | 0.056 |
| 11 | 0.114 |
| 12 | 0.141 |
| 13 | 0.170 |
| 14 | 0.199 |
| 15 | 0.034 |
| 16 | 0.124 |
| 17 | 0.072 |
| 18 | 0.173 |
| 20 | 0.188 |
| 21 | 0.341 |
| 22 | 0.288 |
| 23 | 0.100 |
| 24 | 0.056 |
| 25 | 0.312 |
| 26 | 0.169 |
| 27 | 0.235 |
| 28 | 0.284 |
| 29 | 6.401 |
| 30 | 0.452 |
| 31 | 0.535 |
| 33 | 0.621 |
| 34 | 3.625 |
| 35 | 0.038 |
| 36 | 0.305 |
| 37 | 3.883 |
| 38 | 0.194 |
| 39 | 0.157 |
| 40 | 0.060 |
| 41 | 0.057 |
| 42 | 0.654 |
| 43 | 0.390 |
| 44 | 2.909 |
| 45 | 2.788 |
| 46 | 0.047 |
| 47 | 0.020 |
| 48 | 0.084 |
| 49 | 0.038 |
| 50 | 0.086 |
| 51 | 0.479 |
| 52 | 0.055 |
| 54 | 0.224 |
| 55 | 0.122 |
| 56 | 0.284 |
| 57 | 0.029 |
| 58 | 0.090 |
| 59 | 0.073 |
| 60 | 0.072 |
| 61 | 0.102 |
| 62 | 0.064 |
| 63 | 0.220 |
| 64 | 0.044 |
| 65 | 0.046 |
| 66 | 0.060 |
| 68 | 0.044 |
| 70 | 0.229 |
| 71 | 1.033 |
| 72 | 0.041 |
| 73 | 0.070 |
| 74 | 0.127 |
| 75 | 0.082 |
| 77 | 0.904 |
| 78 | 0.134 |
| 80 | 0.048 |
| 81 | 0.087 |
| 83 | 0.040 |
| 84 | 0.045 |
| 85 | 0.075 |
| 86 | 0.037 |
| 88 | 0.404 |
| 89 | 0.053 |
| 90 | 0.272 |
| 91 | 0.102 |
| 92 | 0.047 |
| 93 | 0.151 |
| 94 | 0.085 |
| 95 | 0.133 |
| 96 | 0.103 |
| 97 | 0.071 |
| 99 | 0.087 |
| 100 | 0.040 |
| 101 | 0.105 |
| 102 | 0.064 |
| 103 | 0.091 |
| 104 | 0.082 |
| 105 | 0.035 |
| 107 | 0.060 |
| 109 | 0.033 |
| 110 | 0.027 |
| 111 | 0.101 |
| 112 | 0.135 |
| 113 | 0.044 |
| 114 | 0.057 |
| 115 | 0.023 |
| 116 | 5.134 |
| 117 | 0.034 |
| 123 | 0.147 |
| 124 | 0.143 |
| 125 | 0.054 |
| 126 | 0.036 |
| 127 | 0.038 |
| 128 | 0.053 |
| 129 | 0.069 |
| 130 | 0.117 |
| 131 | 0.033 |
| 132 | 0.059 |
| 133 | 0.311 |
| 135 | 0.026 |
| 136 | 0.046 |
| 137 | 0.078 |
| 138 | 0.034 |
| 139 | 1.219 |
| 140 | 0.129 |
| 141 | 0.049 |
| 143 | 0.106 |
| 144 | 0.102 |
| 147 | 0.091 |
| 148 | 0.147 |
| 149 | 0.046 |
| 150 | 0.035 |
| 151 | 0.060 |
| 152 | 0.019 |
| 153 | 0.031 |
| 154 | 0.067 |
| 155 | 0.147 |
| 156 | 0.108 |
| 157 | 0.068 |
| 158 | 0.030 |
| 160 | 0.041 |
| 161 | 0.073 |
| 163 | 0.050 |
| 164 | 0.120 |
| 165 | 0.048 |
| 166 | 0.059 |
| 167 | 0.045 |
| 168 | 0.053 |
| 169 | 0.070 |
| 170 | 0.076 |
| 171 | 0.178 |
| 172 | 0.060 |
| 174 | 0.015 |
| 175 | 0.066 |
| 176 | 0.033 |
| 177 | 0.059 |
| 178 | 0.034 |
| 179 | 0.134 |
| 180 | 0.050 |
| 181 | 0.096 |
| 182 | 0.058 |
| 184 | 0.042 |
| 185 | 0.054 |
| 187 | 0.033 |
| 188 | 0.018 |
| 189 | 0.045 |
| 190 | 0.087 |
| 191 | 0.148 |
| 192 | 0.034 |
| 193 | 0.113 |
| 194 | 0.033 |
| 196 | 0.067 |
| 197 | 0.026 |
| 198 | 0.043 |
| 199 | 0.027 |
| 200 | 0.025 |
| 204 | 0.816 |
| 205 | 0.065 |
| 206 | 0.205 |
| 207 | 0.126 |
| 208 | 0.043 |
| 209 | 0.211 |
| 210 | 0.016 |
| 211 | 0.052 |
| 213 | 0.058 |
| 214 | 0.038 |
| 215 | 0.042 |
| 216 | 0.048 |
| 219 | 0.045 |
| 220 | 0.041 |
| 221 | 0.169 |
| 224 | 0.087 |
| 225 | 0.036 |
| 226 | 0.039 |
| 227 | 0.040 |

It will be appreciated from table 1 that the compounds of the invention can bind to the p53 Y220C mutant and restore the ability of the p53 Y220C mutant to bind DNA. Some compounds of the invention exhibited high in vitro DNA binding activity, with EC₅₀ ranging from 0.010 μM to 0.450 μM, preferably from 0.010 μM to 0.090 μM.

### 2. Cell Viability Assay.

Cells were collected and counted. A desired number of cells was seeded into 96-well microplate and cultured in 37° C. cell incubator overnight. Compounds dissolved in cell culture medium were added into cell plate and cultured for 6 days. After balancing CellTiter-Glo reagent and cells at room temperature for 30 min, equal volume of CellTiter-Glo reagents was added to assay plate and shaken for 2 min for cell lysis. The cells were balanced at room temperature for 10 minutes and Luminescence signal was read using Envision. The percent of cell viability for compounds treated wells were normalized between High control and low control. Wells containing cell culture medium and same percentage DMSO sewved as Low control. Wells containing cells and same percentage DMSO sewved as High control. Cell viability (%)=(Luminescence readout_{Compound treated}−Luminescence readout_{Low control})/(Luminescence readout_{High control}−Luminescence readout_{Low control})*100%. Then the data were analyzed either by fitting a 4-parameter logistic model or by Excel to calculate IC₅₀ values. And the IC₅₀ was the concentration at 50% Cell viability on the curve.

The IC₅₀ (μM) values of some example compounds of the present invention weir shown in the following table 2.

TABLE 2

| Comp. | NUGC3 (P53 Y220C) | NUGC4 (P53 WT) |
|---|---|---|
| Ex. No. | IC₅₀ (μM) | IC₅₀ (μM) |
| Ref-1a | 0.54 | 11.04 |
| Ref-1b | 1.92 | 12.46 |
| 6 | 1.19 | 6.83 |
| 10 | 1.68 | 21.02 |
| 15 | 0.36 | 6.99 |
| 41 | 1.54 | 6.14 |
| 46 | 1.35 | 10.47 |
| 52 | 0.75 | 4.58 |
| 57 | 1.11 | 12.04 |
| 59 | 1.44 | 9.82 |
| 60 | 1.95 | 6.10 |
| 61 | 4.53 | 13.03 |
| 62 | 1.43 | 3.70 |
| 64 | 0.98 | 13.60 |
| 65 | 1.56 | 10.03 |
| 66 | 0.82 | 10.32 |
| 68 | 1.08 | 1.39 |
| 72 | 0.43 | 4.43 |
| 73 | 0.77 | 3.42 |
| 74 | 2.05 | 2.73 |
| 75 | 1.02 | 5.34 |
| 80 | 1.21 | 5.53 |
| 81 | 1.68 | 8.64 |
| 83 | 1.31 | 7.96 |
| 84 | 1.42 | 11.49 |
| 85 | 1.54 | 8.24 |
| 86 | 0.79 | 4.56 |
| 89 | 0.82 | 4.54 |
| 91 | 1.80 | 9.41 |
| 92 | 0.99 | 13.43 |
| 94 | 1.84 | 2.75 |
| 96 | 1.68 | 9.93 |
| 97 | 0.96 | 5.72 |
| 99 | 1.44 | 6.58 |
| 101 | 2.05 | 2.05 |
| 102 | 0.81 | 1.92 |
| 103 | 1.28 | 2.65 |
| 104 | 1.38 | 1.75 |
| 105 | 1.83 | 10.03 |
| 106 | 0.78 | 3.80 |
| 107 | 7.25 | 20.57 |
| 109 | 1.19 | 47.68 |
| 110 | 1.02 | 14.19 |
| 111 | 1.84 | 13.13 |
| 112 | 2.09 | 7.51 |
| 113 | 0.93 | 6.10 |
| 114 | 1.24 | 10.48 |
| 115 | 0.24 | 8.58 |
| 117 | 0.93 | 8.54 |
| 124 | 2.31 | 7.23 |
| 125 | 1.27 | 2.57 |
| 126 | 0.63 | 2.74 |
| 127 | 0.33 | 2.26 |
| 128 | 0.87 | 2.26 |
| 129 | 1.85 | 8.95 |
| 130 | 3.55 | 6.85 |
| 131 | 0.66 | 2.54 |
| 132 | 0.82 | 3.08 |
| 135 | 1.16 | 3.62 |
| 136 | 1.63 | 4.88 |
| 138 | 0.88 | 11.11 |
| 141 | 1.13 | 9.06 |
| 143 | 1.94 | 6.94 |
| 147 | 1.47 | 6.83 |
| 148 | 1.56 | 3.08 |
| 149 | 0.37 | 3.70 |
| 150 | 0.39 | 9.92 |
| 152 | 1.02 | 6.69 |
| 153 | 0.37 | 4.07 |
| 154 | 1.89 | 4.19 |
| 157 | 0.53 | 4.84 |
| 158 | 0.37 | 3.95 |
| 160 | 1.41 | >50 |
| 161 | 1.15 | 12.91 |
| 163 | 1.21 | 3.82 |
| 165 | 20.49 | >50 |
| 166 | 1.90 | 7.57 |
| 167 | 1.41 | 2.70 |
| 168 | 1.86 | 7.38 |
| 169 | 1.63 | 4.33 |
| 170 | 1.64 | 3.72 |
| 172 | 1.51 | 7.55 |
| 174 | 0.35 | 5.93 |
| 175 | 3.55 | 35.22 |
| 176 | 0.77 | 2.62 |
| 177 | 1.10 | 1.98 |
| 178 | 0.66 | 1.70 |
| 180 | 0.91 | 5.89 |
| 182 | 2.03 | 3.74 |
| 184 | 0.44 | 4.46 |
| 185 | 0.88 | 2.79 |
| 187 | 0.29 | 5.32 |
| 188 | 1.90 | 40.55 |
| 189 | 3.64 | 8.67 |
| 190 | 0.93 | 7.69 |
| 192 | 1.19 | 7.61 |
| 193 | 1.22 | 18.56 |
| 194 | 1.75 | 12.23 |
| 196 | 6.71 | 18.58 |
| 197 | 0.39 | 11.53 |
| 198 | 0.26 | 5.10 |
| 199 | 0.36 | 49.01 |
| 200 | 1.40 | 13.12 |
| 205 | 1.32 | 4.10 |
| 208 | 2.13 | 13.73 |
| 210 | 1.28 | 12.01 |
| 211 | 1.07 | 11.26 |
| 213 | 0.37 | 9.59 |
| 214 | 0.38 | 13.88 |
| 215 | 0.37 | 37.34 |
| 216 | 0.27 | >50 |
| 217 | 0.32 | 15.42 |
| 218 | 0.60 | 14.63 |
| 219 | 0.36 | 19.46 |
| 220 | 0.25 | 8.11 |
| 225 | 0.44 | 13.59 |
| 226 | 0.32 | 19.35 |
| 227 | 0.28 | 17.62 |

It will be appreciated from table 2 that the compounds of the invention can selectively bind to the p53 Y220C mutant and actually are capable of Y220C mutant-specific p53 reactivation. It can also be seen that some compounds of the invention exhibit an IC₅₀ for NUGC3 (P53 Y220C) at least 2 limes, 5 limes, 10 limes, 20 limes, limes lower than that for NUGC4 (P53 WT). That is to say, the compounds of the invention can selectively and specifically inhibit growth of cells harbouring p53 Y220C mutant, as compared to cells with wild-type p53. Some compounds of the invention exhibited high inhibitory activity in NUGC3 (P53 Y220C) cells, with IC₅₀ ranging from 0.020 μM to 0.950 μM, preferably from 0.10 μM to 0.50 μM.

### 3. Reporter Gene Assay

NUGC-3_P53_Luc single clonal cell line was established using pGL4.38[luc2P/p53 RE/Hygro] Vector (E365A, promega). NUGC-3_P53-Luc single clonal cells were cultured in 37° C. cell incubator before use. NUGC-3-P53-Luc cells were collected and washed twice with PBS to remove phenol red and then resuspended in medium to a proper concentration. Tested compound dilutions were transferred into 384 well assay plates (PerkinElmer). A desired number of cells was seeded into the assay plate and cultured for 6 hours at 37° C. cell incubator. Britelite plus Luciferase Assay Reagent were added into each well and the luminescence value was recorded on Envision. The percent of activity value of compounds treated wells was normalized between high control and low control. Wells containing cells and Ref-1a (10 μM) sewved as high control, and wells containing cells and same percentage of DMSO sewved as low control. Activity value (%)=(Luminescence readout_{Compound treated}−Luminescence readout_{Low control})/(Luminescence readout_{High control}−Luminescence readout_{Low control})*100%. Then the data were analyzed either by fitting a 4-parameter logistic model or by Excel to calculate EC₅₀ values and the EC₅₀ was the concentration at 50% Activity on the curve.

The EQ₅₀ (μM) values of some example compounds of the present invention weir shown in the following table

TABLE 3

| Comp. Ex. No. | p53(Y220C)_NUGC-3 EC₅₀ (μM) |
|---|---|
| Ref-1a | 0.05 |
| Ref-1b | 0.20 |
| 6 | 0.36 |
| 10 | 0.23 |
| 15 | 0.31 |
| 41 | 0.60 |
| 46 | 0.17 |
| 52 | 0.23 |
| 57 | 0.08 |
| 59 | 0.15 |
| 60 | 0.47 |
| 61 | 0.53 |
| 62 | 0.34 |
| 64 | 0.15 |
| 65 | 0.12 |
| 66 | 0.12 |
| 68 | 0.64 |
| 72 | 0.21 |
| 73 | 0.13 |
| 74 | 0.25 |
| 75 | 0.16 |
| 80 | 0.23 |
| 81 | 0.29 |
| 83 | 0.21 |
| 84 | 0.17 |
| 85 | 0.22 |
| 86 | 0.14 |
| 89 | 0.23 |
| 91 | 0.26 |
| 92 | 0.18 |
| 94 | 0.19 |
| 96 | 0.13 |
| 97 | 0.15 |
| 99 | 0.15 |
| 101 | 0.36 |
| 102 | 0.11 |
| 103 | 0.21 |
| 104 | 0.24 |
| 105 | 0.20 |
| 106 | 0.09 |
| 107 | 0.96 |
| 109 | 0.12 |
| 110 | 0.04 |
| 111 | 0.16 |
| 112 | 0.27 |
| 113 | 0.07 |
| 114 | 0.12 |
| 115 | 0.03 |
| 117 | 0.17 |
| 124 | 0.47 |
| 125 | 0.18 |
| 126 | 0.08 |
| 127 | 0.10 |
| 128 | 0.10 |
| 129 | 0.18 |
| 130 | 0.36 |
| 131 | 0.07 |
| 132 | 0.26 |
| 135 | 0.04 |
| 136 | 0.24 |
| 138 | 0.07 |
| 141 | 0.09 |
| 143 | 0.45 |
| 147 | 0.11 |
| 148 | 0.21 |
| 149 | 0.09 |
| 150 | 0.08 |
| 152 | 0.12 |
| 153 | 0.10 |
| 154 | 0.41 |
| 157 | 0.11 |
| 158 | 0.09 |
| 160 | 0.19 |
| 161 | 0.12 |
| 163 | 0.18 |
| 165 | 4.22 |
| 166 | 0.24 |
| 172 | 0.18 |
| 174 | 0.06 |
| 175 | 0.64 |
| 176 | 0.15 |
| 177 | 0.35 |
| 178 | 0.12 |
| 180 | 0.11 |
| 182 | 0.48 |
| 184 | 0.10 |
| 185 | 0.11 |
| 187 | 0.05 |
| 188 | 0.06 |
| 189 | 0.70 |
| 190 | 0.19 |
| 192 | 0.13 |
| 193 | 0.13 |
| 194 | 0.15 |
| 196 | 0.66 |
| 197 | 0.12 |
| 198 | 0.20 |
| 199 | 0.06 |
| 200 | 0.06 |
| 205 | 0.55 |
| 208 | 0.71 |
| 210 | 0.03 |
| 211 | 0.22 |
| 213 | 0.22 |
| 214 | 0.10 |
| 215 | 0.14 |
| 216 | 0.12 |
| 217 | 0.15 |
| 218 | 0.13 |
| 219 | 0.09 |
| 220 | 0.15 |
| 225 | 0.17 |
| 226 | 0.05 |
| 227 | 0.04 |

It will be appreciated from table 3 that the compounds of the invention can enter NUGC-3 cell across membrane, bind to the p53 Y220C mutant in cells and restore the ability of the p53 Y220C mutant to bind DNA. Some compounds of the invention exhibited high DNA binding activity in cells, with EC₅₀ ranging from 0.010 μM to 0.450 μM, preferably from 0.010 μM to 0.090 μM.

### Pharmacokinetic Study

### 1. PK in Rat

### Experimental Methods:

The compounds were dissolved in 10% DMSO, 10% Solutol and 80% PBS to obtain solutions. To SD male rats were administered the compounds of the invention orally and intravenously. Then blood samples weir collected at 0 hour (pre-dose), 0.25, 0.5, 1, 2, 4, 6, 8, 24 hours post-dose. The blood samples were placed in centrifuge tube with ethylenediamine tetracetic acid (EDTA-K₂) as the anticoagulant. After centrifugation at 5000 rpm/min for 10 mins, 100 μL plasma was measured and stored at −80° C. 50 μL plasma and 5 μL fluid infusion (V_{ACN}:VH₂O═1:1) and 200agulant. After centrifugation at 5000 rpm/min for 10 mins, 100 μL plasma was measured and store at 4° C. the supernatant was separated by centrifuging the mixture at 4600 rpm/min. The supernatant and water were mixed together to obtain plasma homogenate samples, with a volume ratio of supernatant to water of 1:2.

### Analysis and Detection:

The reversed-phased liquid chromatography with tandem mass spectrometry (LC-MS/MS) in positive ion mode equipped was used to quantificationally analyze the of compounds in plasma of rat.

Injection volume: 5 μL;

The model of the chromatographic column: Phenomenex Kinetex 5 μm C18(100A (2.1*50) mm) chromatographic column;

The mobile phase A: V_{ACN}:V_{H2O}=5:95, (0.1% HCOOH);

The mobile phase B: V_{ACN}:V_{H2O}=95:5, (0.1% HCOOH);

Flow rate: 0.6 ml/min;

Flow time: 3.00 mins;

The elution gradient is shown in the Table 4:

TABLE 4
The elution gradient of the LCMS/MS

| time(min) | mobile phase A(%) | mobile phase B(%) |
|---|---|---|
| 0.00 | 95 | 5 |
| 0.40 | 95 | 5 |
| 1.20 | 5 | 95 |
| 2.50 | 5 | 95 |
| 2.51 | 95 | 5 |
| 3.00 | 95 | 5 |

The parameters of the mass spectrometer:

Ion source: electrospray ionization (ESI); Ionization mode: positive ion mode; Curtain Gas (CUR): 30.00 Psi; Ion Source Gas1 (GS1): 50.00 Psi; Ion Source Gas2(GS2): 50.00 Psi; IonSpray Voltage (IS): 5500V; Temperature (TEM): 450° C.; Collision Gas (CAD): 8.00 Psi; Interface Heater (ihe) condition: on.

Pharmacokinetic parameters such as CL (mL/min/kg), Vss (L/kg), Cₘₐₓ (ng/mL), AUCₗₐₛₜ (h*ng/mL), F %, or t_{1/2} (h) were calculated using WinNonlin's software with non-compartmental model.

### 2. PK in Mouse

### Experimental Methods

The compounds were dissolved in 10% DMSO, 10% Solutol and 80% PBS to obtain solutions. To Balb/c mouse were administered the compounds of the invention orally and intravenously. Then blood samples were collected at 0.5, 1, 2, 4, 8, 24 hours post-dose. The blood samples were placed in centrifuge tube with ethylenediamine tetracetic acid (EDTA-K₂) as the anticoagulant. After centrifugation at 5000 rpm/min for 10 mins, 40 samples were placed in centrifuge tube 80° C. 30 μL plasma and 3 μL fluid infusion (V_{ACN}:V_{H2O}=1:1) and 150agulant. After centrifugation at 5000 rpmnal standard compound were mixed together, and then at 4° C. the supernatant was separated by centrifuging the mixture at 4600 rpm/min. The supernatant and water were mixed together to obtain plasma homogenate sample, with a volume ratio of supernatant to water of 1:2.

### Analysis and Detection:

The reversed-phased liquid chromatography with tandem mass spectrometry (LC-MS/MS) in positive ion mode equipped was used to quantificationally analyze compounds in plasma of mice.

Injection volume: 5 μL;

The model of the chromatographic column: Phenomenex Kinetex 5 μm C18(100A (2.1*50) mm) chromatographic column;

The mobile phase A: V_{ACN}:V_{H2O}=5:95, (0.1% HCOOH);

The mobile phase B: V_{ACN}:V_{H2O}=95:5, (0.1% HCOOH);

Flow rate: 0.6 ml/min;

Flow time: 3.00 mins;

The elution gradient is shown in the Table 5:

TABLE 5
The elution gradient of the LCMS/MS

| Time (min) | mobile phase A(%) | mobile phase B(%) |
|---|---|---|
| 0.00 | 95 | 5 |
| 0.40 | 95 | 5 |
| 1.20 | 5 | 95 |
| 2.50 | 5 | 95 |
| 2.51 | 95 | 5 |
| 3.00 | 95 | 5 |

The parameters of the mass spectrometer:

Ion source: electrospray ionization (ESI); Ionization mode: positive ion mode; Curtain Gas (CUR): 30.00 Psi; Ion Source Gas1 (GS1): 50.00 Psi; Ion Source Gas2(GS2): 50.00 Psi; IonSpray Voltage (IS): 5500V; Temperature (TEM): 450° C.; Collision Gas (CAD): 8.00 Psi; Interface Heater (ihe) condition: on.

Pharmacokinetic parameters such as CL (mL/min/kg), Vss (L/kg), Cₘₐₓ (ng/mL), AUCₗₐₛₜ (h*ng/mL), F %, or t_{1/2} (h) were calculated using WinNonlin's software with non-compartmental model.

While the invention is described in conjunction with the enumerated embodiments, it will be understood that they are not intended to limit the invention to those embodiments. The invention is intended to cover all alternatives, modifications, and equivalents that may be included within the scope of the present invention. One skilled in the art will recognize many methods and materials similar or equivalent to those described herein, which could be used in the practice of the present invention. The present invention is in no way limited to the methods and materials described. In the event that one or more of the incorporated literature, patents, and similar materials differs from or contradicts this application, including but not limited to defined terms, term usage, described techniques, or the like, this application controls.

All references including patents, patent applications and publications cited in the present application are incorporated herein by reference in their entirety, as if each of them is individually incorporated. Further, it would be appreciated that one skilled in the art could make various changes or modifications to the invention without departing from the scope of the invention defined by the appended claims below. Accordingly, the present invention is not intended to be limited to the disclosed embodiments. Rather the present invention is intended to cover the disclosed embodiments as well as others falling within the scope and spirit of the invention to the fullest extent permitted in view of this disclosure and the inventions defined by the claims appended herein below.

What is claimed is:

1-82. (canceled)

83. A compound of formula (I), or a stereoisomer, tautomer, deuterated derivative, prodrug or pharmaceutically acceptable salt thereof: 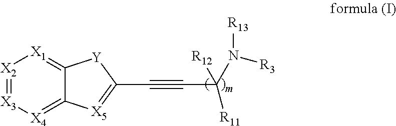
Y is selected from O, S, NR′, S═O, —S(═O)(═NR′)— or O═S═O;
one of X₁, X₂, X₃ and X₄ is selected from CR₂, and the others of X₁, X₂, X₃ and X₄ are each independently selected from N or CR₄;
X₅ is selected from N or CR₁;
R₁ is independently selected from hydrogen, deuterium, halogen, —C₁₋₆alkyl, —C₂₋₆alkenyl, —C₂₋₆alkynyl, —C₁₋₆haloalkyl, —CN, —OR′, —SR′, —C(O)R′, —C(O)N(R′)₂, —C(O)OR′, —OC(O)R′, —OC(O)N(R′)₂, —N(R′)₂, —NR′C(O)R′, —NR′C(O)OR′, —NR′C(O)N(R′)₂, —S(O)R′, —S(O)N(R′)₂, —NR′S(O)R′, —NR′S(O)N(R′)₂, —S(O)₂R′, —S(O)₂N(R′)₂, —NR′S(O)₂R′, —NR′S(O)₂N(R′)₂, —PO(R′)₂, 3-12 membered cycloalkyl, 3-12 membered cycloalkenyl, 3-12 membered heterocycloalkyl, 3-12 membered heterocycloalkenyl, 6-12 membered aryl or 5-12 membered heteroaryl; said —C₁₋₆alkyl, —C₂₋₆alkenyl, —C₂₋₆alkynyl, —C₁₋₆haloalkyl, 3-12 membered cycloalkyl, 3-12 membered cycloalkenyl, 3-12 membered heterocycloalkyl, 3-12 membered heterocycloalkenyl, 6-12 membered aryl, and 5-12 membered heteroaryl are each independently optionally substituted with one or more (such as 1, 2, 3, 4, 5 or 6) substituents selected from deuterium, halogen, —C₁₋₆alkyl, —C₂₋₆alkenyl, —C₂₋₆alkynyl, —C₁₋₆haloalkyl, —CN, oxo, —OR′, —SR′, —C(O)R′, —C(O)N(R′)₂, —C(O)OR′, —OC(O)R′, —OC(O)N(R′)₂, —N(R′)₂, —NR′C(O)R′, —NR′C(O)OR′, —NR′C(O)N(R′)₂, —S(O)R′, —S(O)N(R′)₂, —NR′S(O)R′, —NR′S(O)N(R′)₂, —S(O)₂R′, —S(O)₂N(R′)₂, —NR′S(O)₂R′, —NR′S(O)₂N(R′)₂, —PO(R′)₂, 3-12 membered cycloalkyl, 3-12 membered cycloalkenyl, 3-12 membered heterocycloalkyl, 3-12 membered heterocycloalkenyl, 6-12 membered aryl or 5-12 membered heteroaryl;
R₂ is —NR₅₁R₅₂, —OR₅₃ or —SR₅₄;
R₃ is selected from hydrogen, deuterium, —C₁₋₆alkyl, —C₂₋₆alkenyl, —C₂₋₆alkynyl, —C₁₋₆haloalkyl, —C(O)R′, —C(O)N(R′)₂, —C(O)OR′, —S(O)R′, —S(O)N(R′)₂, —S(O)₂R′, —S(O)₂N(R′)₂, —PO(R′)₂, 3-12 membered cycloalkyl, 3-12 membered cycloalkenyl, 3-12 membered heterocycloalkyl, 3-12 membered heterocycloalkenyl, 6-12 membered aryl, 5-12 membered heteroaryl; said —C₁₋₆alkyl, —C₂₋₆alkenyl, —C₂₋₆alkynyl, —C₁₋₆haloalkyl, 3-12 membered cycloalkyl, 3-12 membered cycloalkenyl, 3-12 membered heterocycloalkyl, 3-12 membered heterocycloalkenyl, 6-12 membered aryl, and 5-12 membered heteroaryl are each independently optionally substituted with one or more (such as 1, 2, 3, 4, 5 or 6) substituents of R₃ₐ;
each R₃ₐ is independently selected from deuterium, halogen, —C₁₋₆alkyl, —C₂₋₆alkenyl, —C₂₋₆alkynyl, —C₁₋₆haloalkyl, —CN, oxo, —OR′, —SR′, —C(O)R′, —C(O)N(R′)₂, —C(O)OR′, —OC(O)R′, —OC(O)N(R′)₂, —N(R′)₂, —NR′C(O)R′, —NR′C(O)OR′, —NR′C(O)N(R′)₂, —S(O)R′, —S(O)N(R′)₂, —NR′S(O)R′, —NR′S(O)N(R′)₂, —S(O)₂R′, —S(O)₂N(R′)₂, —S(═O)(═NR′)R′, —NR′S(O)₂R′, —NR′S(O)₂N(R′)₂, —PO(R′)₂, 3-12 membered cycloalkyl, 3-12 membered cycloalkenyl, 3-12 membered heterocycloalkyl, 3-12 membered heterocycloalkenyl, 6-12 membered aryl, 5-12 membered heteroaryl; said —C₁₋₆alkyl, —C₂₋₆alkenyl, —C₂₋₆alkynyl, —C₁₋₆haloalkyl, 3-12 membered cycloalkyl, 3-12 membered cycloalkenyl, 3-12 membered heterocycloalkyl, 3-12 membered heterocycloalkenyl, 6-12 membered aryl, and 5-12 membered heteroaryl are each independently optionally substituted with one or more substituents R_{3b},
each R_{3b} is independently selected from deuterium, halogen, —C₁₋₆alkyl, —C₂₋₆alkenyl, —C₂₋₆alkynyl, —C₁₋₆haloalkyl, —CN, oxo, —OR′, —SR′, —C(O)R′, —C(O N(R′)₂, —C(O)OR′, —OC(O)R′, —OC(O)N(R′)₂, —N(R′)₂, —NR′C(O)R′, —NR′C(O)OR′, —NR′C(O)N(R′)₂, —S(O)R′, —S(O)N(R′)₂, —NR′S(O)R′, —NR′S(O)N(R′)₂, —S(O)₂R′, —S(O)₂N(R′)₂, —S(═O)(═NR′)R′, —NR′S(O)₂R′, —NR′S(O)₂N(R′)₂, —PO(R′)₂;
R₄ at each occurrence is independently selected from hydrogen, deuterium, halogen, —C₁₋₆alkyl, —C₂₋₆alkenyl, —C₂₋₆alkynyl, —C₁₋₆haloalkyl, —CN, —OR′, —SR′, —C(O)R′, —C(O)N(R′)₂, —C(O)OR′, —OC(O)R′, —OC(O)N(R′)₂, —N(R′)₂, —NR′C(O)R′, —NR′C(O)OR′, —NR′C(O)N(R′)₂, —S(O)R′, —S(O)N(R′)₂, —NR′S(O)R′, —NR′S(O)N(R′)₂, —S(O)₂R′, —S(O)₂N(R′)₂, —NR′S(O)₂R′, —NR′S(O)₂N(R′)₂, —PO(R′)₂, 3-12 membered cycloalkyl, 3-12 membered cycloalkenyl, 3-12 membered heterocycloalkyl, 3-12 membered heterocycloalkenyl, 6-12 membered aryl or 5-12 membered heteroaryl; said —C₁₋₆alkyl, —C₂₋₆alkenyl, —C₂₋₆alkynyl, —C₁₋₆haloalkyl, 3-12 membered cycloalkyl, 3-12 membered cycloalkenyl, 3-12 membered heterocycloalkyl, 3-12 membered heterocycloalkenyl, 6-12 membered aryl, and 5-12 membered heteroaryl are each independently optionally substituted with one or more (such as 1, 2, 3, 4, 5 or 6) substituents selected from deuterium, halogen, —C₁₋₆alkyl, —C₂₋₆alkenyl, —C₂₋₆alkynyl, —C₁₋₆haloalkyl, —CN, oxo, —OR′, —SR′, —C(O)R′, —C(O)N(R′)₂, —C(O)OR′, —OC(O)R′, —OC(O)N(R′)₂, —N(R′)₂, —NR′C(O)R′, —NR′C(O)OR′, —NR′C(O)N(R′)₂, —S(O)R′, —S(O)N(R′)₂, —NR′S(O)R′, —NR′S(O)N(R′)₂, —S(O)₂R′, —S(O)₂N(R′)₂, —S(═O)(═NR′)R′, —NR′S(O)₂R′, —NR′S(O)₂N(R′)₂, —PO(R′)₂, 3-12 membered cycloalkyl, 3-12 membered cycloalkenyl, 3-12 membered heterocycloalkyl, 3-12 membered heterocycloalkenyl, 6-12 membered aryl or 5-12 membered heteroaryl;
R₅₁, R₅₂, R₅₃ and R₅₄ are each independently selected from hydrogen, deuterium, —C₁₋₆alkyl, —C₂₋₆alkenyl, —C₂₋₆alkynyl, —C₁₋₆haloalkyl, —C(O)R′, —C(O)N(R′)₂, —C(O)OR′, —S(O)R′, —S(O)N(R′)₂, —S(O)₂R′, —S(O)₂N(R′)₂, —PO(R′)₂, 3-12 membered cycloalkyl, 3-12 membered cycloalkenyl, 3-12 membered heterocycloalkyl, 3-12 membered heterocycloalkenyl, 6-12 membered aryl, 5-12 membered heteroaryl; said —C₁₋₆alkyl, —C₂₋₆alkenyl, —C₂₋₆alkynyl, —C₁₋₆haloalkyl, 3-12 membered cycloalkyl, 3-12 membered cycloalkenyl, 3-12 membered heterocycloalkyl, 3-12 membered heterocycloalkenyl, 6-12 membered aryl, and 5-12 membered heteroaryl are each independently optionally substituted with one or more (such as 1, 2, 3, 4, 5 or 6) substituents selected from deuterium, halogen, —C₁₋₆alkyl, —C₂₋₆alkenyl, —C₂₋₆alkynyl, —C₁₋₆haloalkyl, —CN, oxo, ═NR′, —C₁₋₆alkyl-CH(R′)₂, —OR′, —SR′, —C(O)R′, —C(O)N(R′)₂, —C(O)OR′, —OC(O)R′, —OC(O)N(R′)₂, —N(R′)₂, —NR′C(O)R′, —NR′C(O)OR′, —NR′C(O)N(R′)₂, —S(O)R′, —S(O)N(R′)₂, —NR′S(O)R′, —NR′S(O)N(R′)₂, —S(O)₂R′, —S(O)₂N(R′)₂, —S(═O)(═NR′)R′, —NR′S(O)₂R′, —NR′S(O)₂N(R′)₂, —PO(R′)₂, 3-12 membered cycloalkyl, 3-12 membered cycloalkenyl, 3-12 membered heterocycloalkyl, 3-12 membered heterocycloalkenyl, 6-12 membered aryl, or 5-12 membered heteroaryl;
R₁₁ and R₁₂ are independently selected from hydrogen, deuterium, —OH, halogen, —CN, oxo, —C₁₋₆ alkyl, —C₁₋₆ haloalkyl, —C₁₋₆alkoxy, —NH₂, —NHC₁₋₆alkyl, —N(C₁₋₆alkyl)₂ or 3-6 membered cycloalkyl;
wherein said —C₁₋₆alkyl, —C₁₋₆alkoxy and 3-6 membered cycloalkyl are each independently optionally substituted with one or more (such as 1, 2, 3, 4, 5 or 6) substituents selected from deuterium, —OH, halogen, —CN, oxo, —C₁₋₆alkoxy, —NH—C₁₋₆alkyl, —N(C₁₋₄ alkyl)₂, or 3-6 membered cycloalkyl;
R₁₃ is selected from hydrogen, deuterium, —C₁₋₆alkyl or 3-6 membered cycloalkyl; wherein said —C₁₋₆ alkyl and 3-6 membered cycloalkyl are each independently optionally substituted with one or more (such as 1, 2, 3, 4, 5 or 6) substituents selected from deuterium, —OH, halogen, —CN, oxo, —C₁₋₆alkoxy, —NH₂, —NC₁₋₆alkyl, or —N(C₁_₄ alkyl)₂;
each R′ at each occurrence is independently selected from hydrogen, deuterium, halogen, —OH, —CN, oxo, —NH₂, —NHC₁₋₆ alkyl, —N(C₁₋₆ alkyl)₂, —C₁₋₆alkyl, —C₁₋₆alkylOC₁₋₆alkyl, —C₁₋₆alkyl-NHC₁₋₆alkyl, —C₁₋₆alkyl-N(C₁₋₆alkyl)₂, —C₁₋₆haloalkyl, —OC₁₋₆alkyl, —C₃₋₁₄cycloalkyl, —C₃₋₁₄heterocycloalkyl, —C₂₋₆alkenyl, —C₂₋₆alkynyl, 6-12 membered aryl, or 5-12 membered heteroaryl; wherein said —C₁₋₆alkyl, —OC₁₋₆alkyl, —C₃₋₁₄cycloalkyl, —C₃₋₁₄heterocycloalkyl, —C₂₋₆alkenyl, —C₂₋₆alkynyl, 6-12 membered aryl, or 5-12 membered heteroaryl is independently optionally substituted with 1, 2, 3, 4, 5 or 6 substituents selected from deuterium, halogen, —CN, —C₁₋₃alkyl, oxo, —OH, —OC₁₋₃alkyl, —NH₂, —NHC₁₋₃alkyl, —N(C₁₋₃alkyl)₂, —C(═O)NH₂, —C(═O)NH(C₁₋₃alkyl), —C(═O)N(C₁₋₃alkyl)₂, —S(═O)₂NH₂, —S(═O)₂NH(C₁₋₃alkyl), —S(═O)₂C₁₋₃alkyl, —S(═O)₂N(C₁₋₃alkyl)₂, —S(═O)(═NH)C₁₋₃alkyl, —S(═O)(═NC₁₋₃alkyl)C₁₋₃alkyl, or 3-6 membered cycloalkyl;
said heterocycloalkyl, heterocycloalkenyl, and heteroaryl each independently contains 1, 2 or 3 heteroatoms selected from N, O, P or S;
m is selected from 1, 2, 3, 4, 5 or 6.

84. The compound according to claim 83, wherein,
the formula (I) is: 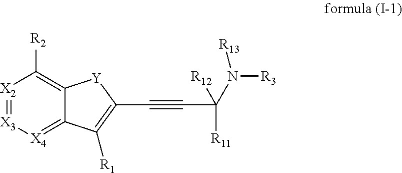
X₂, X₃, and X₄ in the formula (I-1) are each independently selected from N or CR₄;
Y is selected from O, S, S═O, or O═S═O;
or the formula (I) is: 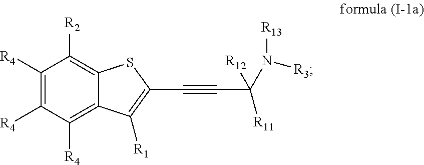
or the formula (I) is: 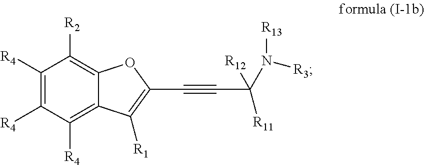
or the formula (I) is: 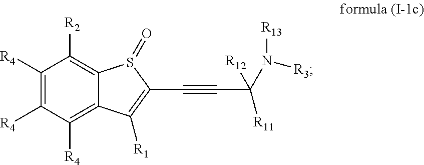
or the formula (I) is: 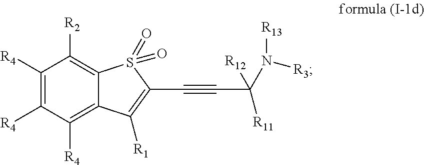
or the formula (I) is: 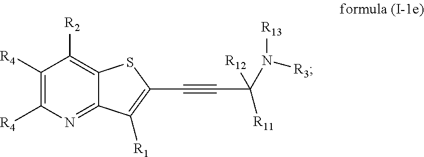
or the formula (I) is: 4 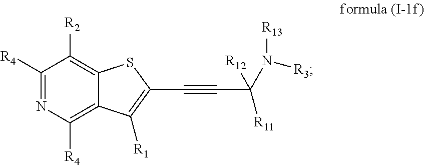
or the formula (I) is: 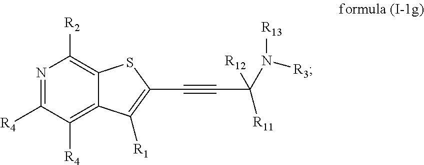
or the formula (I) is selected from:

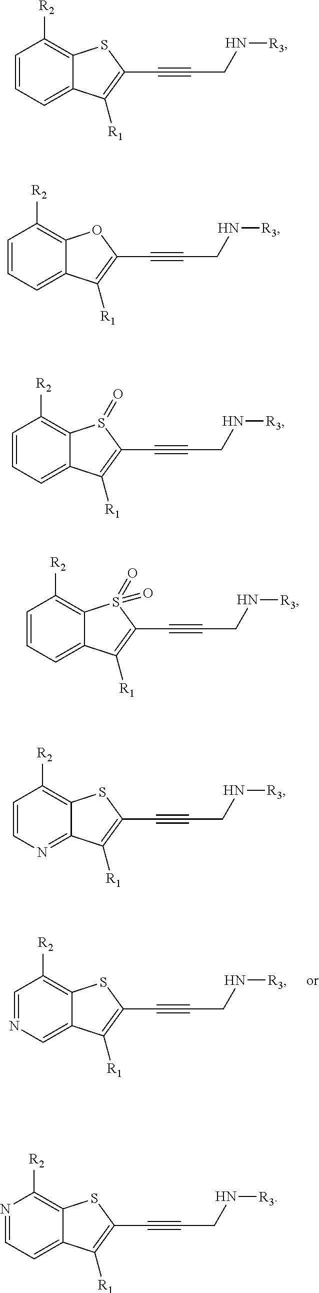

85. The compound according to claim 83, wherein, R₁ is independently selected from halogen, —C₁₋₆alkyl, —C₂₋₆alkenyl, —C₂₋₆alkynyl, —C₁₋₆haloalkyl, —CN, 3-12 membered cycloalkyl, 3-12 membered cycloalkenyl, 3-12 membered heterocycloalkyl, 3-12 membered heterocycloalkenyl, 6-10 membered aryl, or 5-12 membered heteroaryl, said —C₁₋₆alkyl, —C₂₋₆alkenyl, —C₂₋₆alkynyl, —C₁₋₆haloalkyl, 3-12 membered cycloalkyl, 3-12 membered cycloalkenyl, 3-12 membered heterocycloalkyl, 3-12 membered heterocycloalkenyl, 6-10 membered aryl, and 5-12 membered heteroaryl are independently optionally substituted with one or more (such as 1, 2, 3, 4, 5 or 6) substituents selected from halogen, —C₁₋₆alkyl, —C₂₋₆alkenyl, —C₂₋₆alkynyl, —C₁₋₆haloalkyl, —CN, oxo, 3-12 membered cycloalkyl, 3-12 membered cycloalkenyl, 3-12 membered heterocycloalkyl, 3-12 membered heterocycloalkenyl, 6-10 membered aryl, or 5-12 membered heteroaryl, said heterocycloalkyl, heterocycloalkenyl, and heteroaryl each independently contains 1 or 2 heteroatoms selected from N or S;
R₂ is —NR₅₁R₅₂;
R₅₁ is selected from —C₁₋₆alkyl, —C₂₋₆alkenyl, —C₂₋₆alkynyl, —C₁₋₆haloalkyl, —C(O)R′, —C(O)N(R′)₂, —C(O)OR′, 3-6 membered cycloalkyl, 3-6 membered heterocycloalkyl, phenyl, or 5-6 membered heteroaryl; said —C₁₋₆alkyl, —C₂₋₆alkenyl, —C₂₋₆alkynyl, —C₁₋₆haloalkyl, 3-6 membered cycloalkyl, 3-6 membered heterocycloalkyl, phenyl, 5-6 membered heteroaryl are each independently optionally substituted with one or more (such as 1, 2, 3, 4, 5 or 6) substituents selected from halogen, —C₁₋₆alkyl, —C₂₋₆alkenyl, —C₂₋₆alkynyl, —C₁₋₆haloalkyl, —CN, oxo, ═NR′, —C₁₋₆alkyl-CH(R′)₂, —OR′, —SR′, —C(O)R′, —C(O)N(R′)₂, —C(O)OR′, —OC(O)R′, —OC(O)N(R′)₂, —N(R′)₂, —NR′C(O)R′, —NR′C(O)OR′, —NR′C(O)N(R′)₂, 3-6 membered cycloalkyl, 3-6 membered heterocycloalkyl, phenyl, or 5-6 membered heteroaryl, wherein said heterocycloalkyl, heterocycloalkenyl, and heteroaryl each independently contains 1 heteroatom selected from N, O, or S; or
R₃ is independently selected from hydrogen, deuterium, halogen, —C₁₋₆alkyl, —C₂₋₆alkenyl, —C₂₋₆alkynyl, —C₁₋₆haloalkyl, —CN, —NO₂, —OR′, —SR′, —C(O)R′, —C(O)N(R′)₂, —C(O)OR′, —OC(O)R′, —N(R′)₂, —NR′C(O)R′, —S(O)R′, —NR′S(O)R′, —S(O)N(R′)₂, —S(O)₂R′, —NR′S(O)₂R′, —S(O)₂N(R′)₂, 3-12 membered cycloalkyl, 3-12 membered cycloalkenyl, 3-12 membered heterocycloalkyl, 3-12 membered heterocycloalkenyl, 6-10 membered aryl, or 5-12 membered heteroaryl; said —C₁₋₆alkyl, —C₂₋₆alkenyl, —C₂₋₆alkynyl, —C₁₋₆haloalkyl, 3-12 membered cycloalkyl, 3-12 membered cycloalkenyl, 3-12 membered heterocycloalkyl, 3-12 membered heterocycloalkenyl, 6-10 membered aryl, or 5-12 membered heteroaryl at each occurrence is independently optionally substituted with one or more substituents selected from halogen, NH₂, NH—C₁₋₆alkyl, —C₁₋₆alkyl, —C₂₋₆alkenyl, —C₂₋₆alkynyl, —C₁₋₆haloalkyl, —CN, —NO₂, —OR′, —SR′, —C(O)R′, oxo, —C(O)N(R′)₂, —C(O)OR′, —OC(O)R′, —N(R′)₂, —NR′C(O)R′, —S(O)R′, —NR′S(O)R′, —S(O)N(R′)₂, —S(O)₂R′, —NR′S(O)₂R′, —S(O)₂N(R′)₂, —S(═O)(═NR′)R′, 3-12 membered cycloalkyl, 3-12 membered cycloalkenyl, 3-12 membered heterocycloalkyl, 3-12 membered heterocycloalkenyl, 6-10 membered aryl, or 5-12 membered heteroaryl, said heterocycloalkyl, heterocycloalkenyl, and heteroaryl each independently contains 1 heteroatom selected from N, O, or S.

86. The compound according to claim 83, wherein, R₁ is independently selected from —F, —Cl, —C₁₋₃alkyl, —C₂₋₄alkenyl, —C₂₋₄alkynyl, —C₁₋₃haloalkyl, —CN, 3-6 membered cycloalkyl, 3-6 membered heterocycloalkyl, 6-10 membered aryl, 5-12 membered heteroaryl, said —C₁₋₃alkyl, —C₂₋₄alkenyl, —C₂₋₄alkynyl, —C₁₋₃haloalkyl, 3-6 membered cycloalkyl, 3-6 membered heterocycloalkyl, 6-10 membered aryl, and 5-12 membered heteroaryl are independently optionally substituted with one or more (such as 1, 2, 3, 4, 5 or 6) substituents selected from —F, —Cl, —C₁₋₃alkyl, —C₂₋₄alkenyl, —C₂₋₄alkynyl, —C₁₋₃haloalkyl, —CN, oxo, 3-6 membered cycloalkyl, 3-6 membered heterocycloalkyl, 6-10 membered aryl, or 5-12 membered heteroaryl, said heterocycloalkyl, heterocycloalkenyl, and heteroaryl each independently contains 1 or 2 heteroatoms selected from N or S;
R₂ is —NHR₅₁;
R₅₁ is selected from —C1-3alkyl, cyclopentyl, cyclohexyl, 5 membered heterocyclyl containing 1 heteroatom selected from N, O, S, S(═O), S(═O)(═NH) or S(═O)₂ or 6 membered heterocyclyl containing 1 heteroatom selected from N, O, S, S(═O), S(═O)(═NH) or S(═O)₂; said —C₁₋₃alkyl, cyclopentyl, cyclohexyl, 5 membered heterocyclyl and 6 membered heterocyclyl are each independently optionally substituted with 1, 2 or 3 substituents selected from —F, —C₁₋₃alkyl, —OC₁₋₃alkyl, —NH₂, —NHC₁₋₃alkyl, oxo, ═NH, —N(C₁₋₃alkyl)₂, —CN or 3-6 membered cycloalkyl; or
R₃ is independently selected from phenyl, or 5-6 membered heteroaryl containing 1 or 2 heteroatoms selected from N, O or S; said phenyl and 5-6 membered heteroaryl are independently optionally substituted with 1, 2, 3 substituents selected from R_{3c};
R_{3c} is selected from —F; —C₁₋₃alkyl; —OC₁₋₃alkyl; —OC₃₋₆cycloalkyl; —NH₂; —NHC₁₋₃alkyl; —N(C₁₋₃alkyl)₂; —C(═O)NH₂, —C(═O)NH(C₁₋₃alkyl); —C(═O)N(C₁₋₃alkyl)₂; —CN; —S(═O)₂NH₂; —S(═O)₂NH(C₁₋₃alkyl); —S(═O)₂NHC(═O)C₁₋₃alkyl; —S(═O)₂N(C₁₋₃alkyl)₂; —S(═O)₂C₁₋₃alkyl; —S(═O)(═NH)C₁₋₃alkyl; —S(═O)(═NH)C₃₋₆cycloalkyl; —S(═O)(═NC₃₋₆cycloalkyl)C₁₋₃alkyl; —S(═O)(═NC₂₋₆heterocycloalkyl)C₁₋₃alkyl; —S(═O)(═NC₁₋₃alkyl)C₁₋₃alkyl; —S(═O)(═NCN)C₁₋₃alkyl; or 3-6 membered cycloalkyl; wherein said C₁₋₃alkyl, C₃₋₆cycloalkyl, C₂₋₆heterocycloalkyl or OC₁₋₃alkyl at each occurrence is independently optionally substituted with 1, 2, 3, 4, 5 or 6 substituents selected from deuterium, —F, —C₁₋₃alkyl, oxo, —OH, —OC₁₋₃alkyl, —NH₂, —NHC₁₋₃alkyl, —N(C₁₋₃alkyl)₂, —C(═O)NH₂, —C(═O)NH(C₁₋₃alkyl), —C(═O)N(C₁₋₃alkyl)₂, —CN, —S(═O)₂NH₂, —S(═O)₂NH(C₁₋₃alkyl), —S(═O)₂C₁₋₃alkyl, —S(═O)₂N(C₁₋₃alkyl)₂, —S(═O)(═NH)C₁₋₃alkyl, —S(═O)(═NC₁₋₃alkyl)C₁₋₃alkyl, or 3-6 membered cycloalkyl.

87. The compound according to claim 83, wherein, R₁ is independently selected from —F, —Cl, —C₁₋₃alkyl, —C₁₋₃haloalkyl, 3-6 membered cycloalkyl, 3-6 membered heterocycloalkyl, phenyl, or 5-6 membered heteroaryl, said —C₁₋₃alkyl, —C₁₋₃haloalkyl, 3-6 membered cycloalkyl, 3-6 membered heterocycloalkyl, phenyl, or 5-6 membered heteroaryl are independently optionally substituted with one or more (such as 1, 2, 3, 4, 5 or 6) substituents selected from —F, —Cl, —C₁₋₃alkyl, —C₁₋₃haloalkyl, 3-6 membered cycloalkyl, 3-6 membered heterocycloalkyl, phenyl, or 5-6 membered heteroaryl, said heterocycloalkyl, heterocycloalkenyl, and heteroaryl each independently contains 1 or 2 heteroatoms selected from N or S;
R₅₁ is selected from —C₁₋₆alkyl, 5 membered cycloalkyl, 5 membered heterocycloalkyl, 6 membered cycloalkyl, or 6 membered heterocycloalkyl; said —C₁₋₆alkyl, cycloalkyl, and heterocycloalkyl are independently optionally substituted with one or more (such as 1, 2, 3, 4, 5 or 6) substituents selected from —F, —Cl, —CH₃, —CD₃, —CH₂CH₃, —CH₂CH₂CH₃, —CH(CH₃)₂, —CH(OH)CH₂(OH), —CH(OCH₃)CH₂(OH), —CH(OH)CH₂(OCH₃), —CH₂CH(OH)(OCH₃), —CH₂CH(OH)(OCH₂CH₃), —CH₂CH(OCH₃)₂, —OH, —O—CH₃, —O—CH₂CH₃, —O—CH₂CH₂CH₃, —O—CH(CH₃)₂, —C(O)—(CH₂)—NH₂, —C(O)—(CH₂)—NH(CH₃), —C(O)—(CH₂)—NH(CH₂CH₃), —C(O)—(CH₂)—N(CH₃)₂, —C(O)—(CH₂CH₂)—NH₂, —C(O)—(CH₂CH₂)—NH(CH₃), —C(O)—(CH₂CH₂)—NH(CH₂CH₃), —C(O)—(CH₂CH₂)—N(CH₃)₂, —NH₂, —NH(CH₃), —NH(CH₂CH₃), —N(CH₃)₂, —NH(CH₂CH₂CH₃), —NH(CH(CH₃)₂), or —N(CH₃)(CH₂CH₃), said heterocycloalkyl, heterocycloalkenyl, and heteroaryl each independently contains 1 heteroatom selected from N, O, or S; or
R₃ is independently selected from phenyl, 5 membered heteroaryl, or 6 membered heteroaryl; said phenyl and heteroaryl at each occurrence are independently optionally substituted with one or more (such as 1, 2, 3, 4, 5 or 6) substituents selected from —F, —Cl, —CH₃, —CD₃, —CH₂CH₃, —CH₂CH₂CH₃, —CH(CH₃)₂, —CH₂F, —CHF₂, —CF₃, —CH₂CH₂F, —CH₂CHF₂, —CH₂CF₃, —CHFCH₃, —CF₂CH₃, —CHFCH₂F, —CH₂CHFCH₃, —CH₂CF₂CH₃, —CH₂CH₂CF₃, —C(CH₃)₂F, —CN, —OH, —O—CH₃, —O—CH₂CH₃, —O—CH₂CH₂CH₃, —O—CH(CH₃)₂, —SH, —S—CH₃, —S—CH₂CH₃, —S—CH₂CH₂CH₃, —S—CH(CH₃)₂, —CHO, —C(O)—CH₃, —C(O)—CH₂CH₃, —C(O)—CH₂CH₂CH₃, —C(O)—CH(CH₃)₂, —C(O)NH₂, —C(O)NH(CH₃), —C(O)NH(CH₂CH₃), —C(O)N(CH₃)₂, —C(O)NH(CH₂CH₂CH₃), —C(O)NH(CH(CH₃)₂), —C(O)N(CH₃)(CH₂CH₃), —NH₂, —NH(CH₃), —NH(CH₂CH₃), —N(CH₃)₂, —NH(CH₂CH₂CH₃), —NH(CH(CH₃)₂), —N(CH₃)(CH₂CH₃), —NHC(O)(CH₃), —NHC(O)(CH₂CH₃), —NHC(O)(CH₃)₂, —NHC(O)(CH₂CH₂CH₃), —NHC(O)(CH(CH₃)₂), —NHC(O)(CH₃)(CH₂CH₃), —S(O)₂H, —S(O)₂(CH₃), —S(O)₂(CH₂CH₃), —S(O)₂(CH₃)₂, —S(O)₂(CH₂CH₂CH₃), —S(O)₂(CH(CH₃)₂), —S(O)₂(CH₃)(CH₂CH₃), —S(O)₂NH₂, —S(O)₂NH(CH₃), —S(O)₂NH(CH₂CH₃), —S(O)₂N(CH₃)₂, —S(O)₂NH(CH₂CH₂CH₃), —S(O)₂NH(CH(CH₃)₂), —S(O)₂N(CH₃)(CH₂CH₃), —S(═O)(═NH)CH₃, or —S(═O)(═NCH₃)CH₃, said heterocycloalkyl, heterocycloalkenyl, and heteroaryl each independently contains 1 heteroatom selected from N, O, or S.

88. The compound according to claim 8, wherein R₁ is independently selected from —F, —Cl, —C₁₋₃alkyl, —C₁₋₃haloalkyl, 3-6 membered cycloalkyl, 5 membered heteroaryl containing 1 or 2 heteroatoms selected from N, O or S, or 6 membered heteroaryl containing 1 or 2 heteroatoms selected from N; said —C₁₋₃alkyl, —C₁₋₃haloalkyl, 3-6 membered cycloalkyl, 5 membered heteroaryl and 6 membered heteroaryl are each independently optionally substituted with 1, 2 or 3 substituents selected from —F, —C₁₋₃alkyl, —OC₁₋₃alkyl, —NH₂, —NHC₁₋₃alkyl, —N(C₁₋₃alkyl)₂, —CN or 3-6 membered cycloalkyl;
R₅₁ is selected from 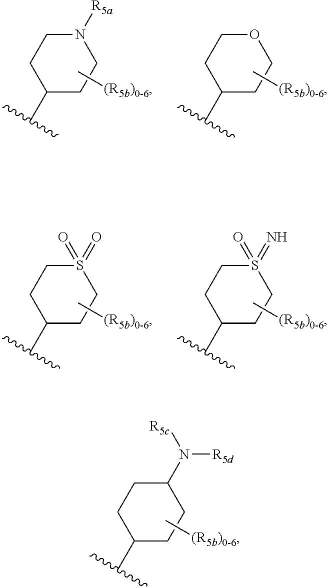
or —C₁₋₆alkyl, said —C₁₋₆alkyl is optionally substituted with 1, 2, 3, 4, 5, or 6 R₅ₑ;
R₅ₐ, R_{5c} and R_{5d} are each independently selected from hydrogen; —C₁₋₆alkyl; or —C₁₋₆alkyl substituted with 1, 2, 3, 4, 5 or 6 substituents selected from —F, —C₁₋₃alkyl, oxo, —OC₁₋₃alkyl, —NH₂, —NHC₁₋₃alkyl, —N(C₁₋₃alkyl)₂, —CN or 3-6 membered cycloalkyl;
R_{5b} and R₅ₑ are each independently selected from —F, —C₁₋₆alkyl, oxo, —OC₁₋₆alkyl, —NH₂, —NHC₁₋₆alkyl, —N(C₁₋₆alkyl)₂, —CN or 111 membered cycloalkyl, wherein, said —C₁₋₆alkyl is optionally substituted with 1, 2, 3 or 4 substituents selected from —F, —C₁₋₃alkyl, oxo, —OC₁₋₃alkyl, —NH₂, —NHC₁₋₃alkyl, —N(C₁₋₃alkyl)₂, —CN or 3-6 membered cycloalkyl; or
R₃ is independently selected from phenyl, 5 membered heteroaryl, or 6 membered heteroaryl; said phenyl and heteroaryl at each occurrence is independently optionally substituted with one or more (such as 1, 2, 3, 4, 5 or 6) substituents selected from —F, —CH₃, —CD₃, —CH₂CH₃, —CH₂CH₂CH₃, —CH(CH₃)₂, —CH₂F, —CF₃, —CH₂CH₂F, —CHFCH₃, —CF₂CH₃, —C(CH₃)₂F, —OH, —O—CH₃, —O—CH₂CH₃, —O—CH₂CH₂CH₃, —O—CH(CH₃)₂, —C(O)NH₂, —C(O)NH(CH₃), —C(O)NH(CH₂CH₃), —C(O)N(CH₃)₂, —C(O)NH(CH₂CH₂CH₃), —C(O)NH(CH(CH₃)₂), —C(O)N(CH₃)(CH₂CH₃), —NH₂, —NH(CH₃), —NH(CH₂CH₃), —N(CH₃)₂, —NH(CH₂CH₂CH₃), —NH(CH(CH₃)₂), —N(CH₃)(CH₂CH₃), —S(O)₂H, —S(O)₂(CH₃), —S(O)₂(CH₂CH₃), —S(O)₂(CH₃)₂, —S(O)₂(CH₂CH₂CH₃), —S(O)₂(CH(CH₃)₂), —S(O)₂(CH₃)(CH₂CH₃), —S(O)₂NH₂, —S(O)₂NH(CH₃), —S(O)₂NH(CH₂CH₃), —S(O)₂N(CH₃)₂, —S(O)₂NH(CH₂CH₂CH₃), —S(O)₂NH(CH(CH₃)₂), —S(O)₂N(CH₃)(CH₂CH₃), —S(═O)(═NH)CH₃, or —S(═O)(═NCH₃)CH₃, said heterocycloalkyl, heterocycloalkenyl, and heteroaryl each independently contains 1 heteroatom selected from N, O, or S.

89. The compound according to claim 83, wherein R₁ is independently selected from —C₁₋₃ alkyl; —C₁₋₃haloalkyl; 5 membered heteroaryl containing 1 or 2 heteroatoms selected from N, O or S; or —C₁₋₃alkyl substituted with 1, 2 or 3 substituents selected from —F, —OC₁₋₃alkyl, —NH₂, —NHC₁₋₃alkyl, N(C₁₋₃alkyl)₂, —CN or 3-6 membered cycloalkyl;
R₅₁ is selected from 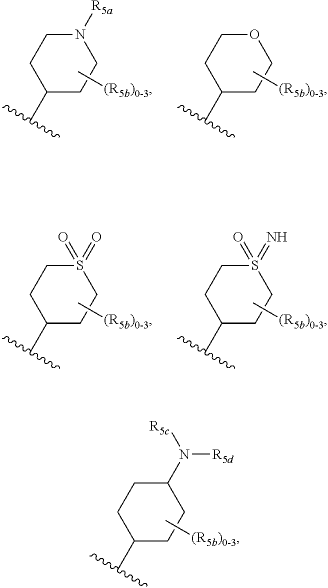
or —C₁₋₃alkyl-N(C₁₋₃alkyl)₂;
R₅ₐ is independently selected from —C₁₋₃alkyl; or —C₁₋₃alkyl substituted with 1, 2, 3, 4, 5 or 6 substituents selected from —F, —C₁₋₃alkyl, oxo, —OC₁₋₃alkyl, —NH₂, —NHC₁₋₃alkyl, —N(C₁₋₃alkyl)₂, —CN or 3-6 membered cycloalkyl;
R_{5b} is independently selected from —F, —C₁₋₃alkyl, oxo, —OC₁₋₃alkyl, —NH₂, —NHC₁₋₃alkyl, —N(C₁₋₃alkyl)₂ or —CN;
R_{5c} and R_{5d} are each independently selected from —C₁₋₃alkyl; or
R₃ is independently selected from

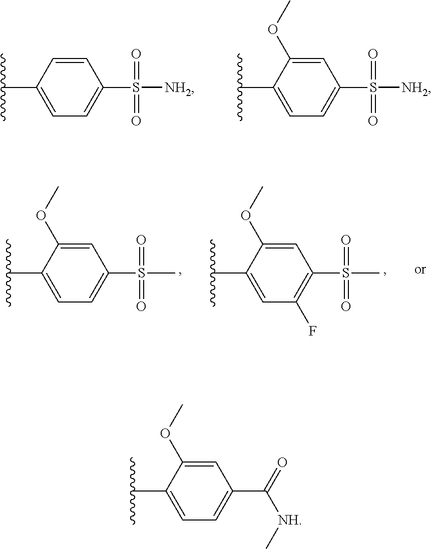

90. The compound according to claim 83, wherein, R₁ is independently selected from 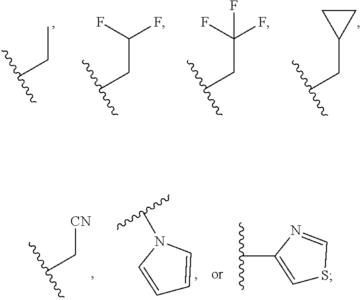
R₅₁ is selected from 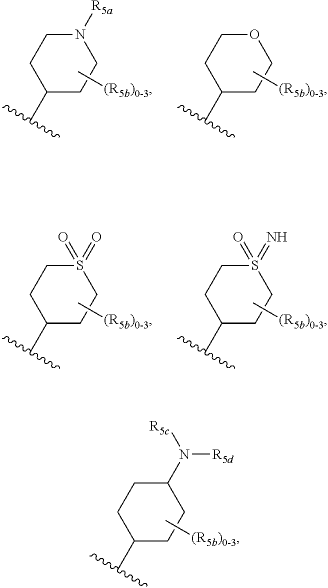
or —CH₂CH₂—N(CH₃)₂;
R₅ₐ is independently selected from methyl, —CH₂CH(OH)OCH₃ or —C(═O)CH₂N(CH₃)₂;
R_{5b} is independently selected from —F;
R_{5c} and R_{5d} are each independently selected from methyl; or
R₃ is independently selected from phenyl or pyridinyl.

91. The compound according to claim 83, wherein,
R₅₁ is selected from 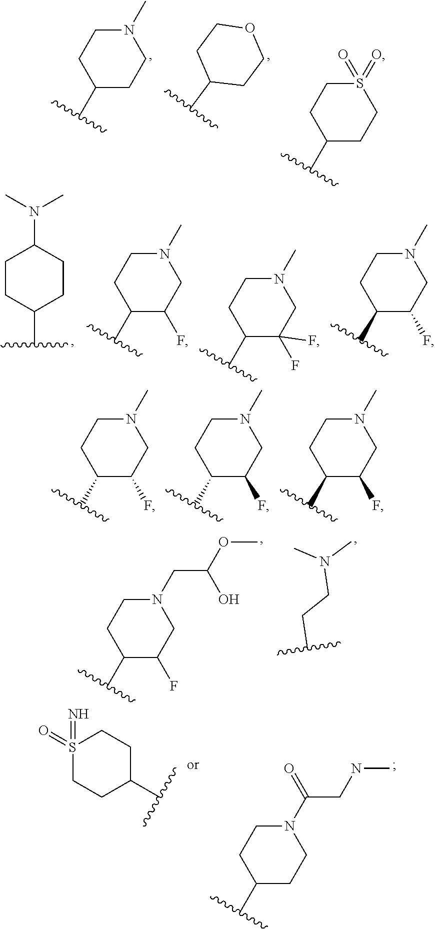
both of R₁ and R₁₂ are hydrogen; or
R₁₃ is H.

92. The compound according to claim 83, wherein the formula I is: 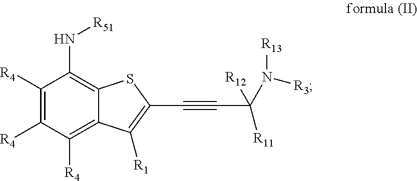
wherein,
R₅₁ is selected from —C₁₋₃alkyl, cyclopentyl, cyclohexyl, 5 membered heterocyclyl containing 1 heteroatom selected from N, O, S, S(═O), S(═O)(═NH) or S(═O)₂ or 6 membered heterocyclyl containing 1 heteroatom selected from N, O, S, S(═O), S(═O)(═NH) or S(═O)₂; said —C₁₋₃alkyl, cyclopentyl, cyclohexyl, 5 membered heterocyclyl and 6 membered heterocyclyl are each independently optionally substituted with 1, 2 or 3 substituents selected from —F, —C₁₋₃alkyl, —OC₁₋₃alkyl, —NH₂, —NHC₁₋₃alkyl, oxo, —N(C₁₋₃alkyl)₂, —CN or 3-6 membered cycloalkyl;
R₁ is independently selected from —F, —Cl, —C₁₋₃alkyl, —C₂₋₄alkenyl, —C₂₋₄alkynyl, —C₁₋₃haloalkyl, —CN, 3-6 membered cycloalkyl, 3-6 membered heterocycloalkyl, 6-10 membered aryl, 5-12 membered heteroaryl, said —C₁₋₃alkyl, —C₂₋₄alkenyl, —C₂₋₄alkynyl, —C₁₋₃haloalkyl, 3-6 membered cycloalkyl, 3-6 membered heterocycloalkyl, 6-10 membered aryl, and 5-12 membered heteroaryl are independently optionally substituted with one or more (such as 1, 2, 3, 4, 5 or 6) substituents selected from —F, —Cl, —C₁₋₃ alkyl, —C₂₋₄alkenyl, —C₂₋₄alkynyl, —C₁₋₃haloalkyl, —CN, oxo, 3-6 membered cycloalkyl, 3-6 membered heterocycloalkyl, 6-10 membered aryl, or 5-12 membered heteroaryl, said heterocycloalkyl, heterocycloalkenyl, and heteroaryl each independently contains 1 or 2 heteroatoms selected from N or S;
each of R₄ is independently selected from hydrogen, deuterium, —F, —Cl, —C₁₋₃alkyl, —OC₁₋₃alkyl, —NH₂, —NHC₁₋₃alkyl, —N(C₁₋₃alkyl)₂, —CN or 3-6 membered cycloalkyl;
R₁₁ and R₁₂ are independently selected from hydrogen, deuterium, —F, —C₁₋₃alkyl, oxo, —OC₁₋₃alkyl, —NH₂, —NHC₁₋₃alkyl, —N(C₁₋₃alkyl)₂, —CN or 3-6 membered cycloalkyl, wherein, said —C₁₋₃alkyl is optionally substituted with 1, 2, 3 or 4 substituents selected from —F, —C₁₋₃alkyl, oxo, —OC₁₋₃alkyl, —NH₂, —NHC₁₋₃alkyl, —N(C₁₋₃alkyl)₂, —CN or 3-6 membered cycloalkyl;
R₁₃ is selected from hydrogen; —C₁₋₃alkyl; or —C₁₋₃alkyl substituted with 1, 2, 3, 4, 5 or 6 substituents selected from —F, —C₁₋₃alkyl, oxo, —OC₁₋₃alkyl, —NH₂, —NHC₁₋₃alkyl, —N(C₁₋₃alkyl)₂, —CN or 3-6 membered cycloalkyl;
R₃ is independently selected from phenyl, or 5-6 membered heteroaryl containing 1 or 2 heteroatoms selected from N, O or S; said phenyl and 5-6 membered heteroaryl are independently optionally substituted with 1, 2, or 3 substituents selected from R_{3c};
R_{3c} is selected from —F; —C₁₋₃alkyl; —OC₁₋₃alkyl; —NH₂; —NHC₁₋₃alkyl; —N(C₁₋₃alkyl)₂; —C(═O)NH₂, —C(═O)NH(C₁₋₃alkyl); —C(═O)N(C₁₋₃alkyl)₂; —CN; —S(═O)₂NH₂; —S(═O)₂NH(C₁₋₃alkyl); —S(═O)₂N(C₁₋₃alkyl)₂; 3-6 membered cycloalkyl; or —C₁₋₃alkyl substituted with 1, 2, 3, 4, 5 or 6 substituents selected from —F, —C₁₋₃alkyl, oxo, —OC₁₋₃alkyl, —NH₂, —NHC₁₋₃alkyl, —N(C₁₋₃alkyl)₂, —C(═O)NH₂, —C(═O)NH(C₁₋₃ alkyl), —C(═O)N(C₁₋₃alkyl)₂, —CN, —S(═O)₂NH₂, —S(═O)₂NH(C₁₋₃alkyl), —S(═O)₂N(C₁₋₃alkyl)₂, —S(═O)(═NH)C₁₋₃alkyl, —S(═O)(═NC₁₋₃alkyl)C₁₋₃alkyl, or 3-6 membered cycloalkyl.

93. The compound according to claim 92, wherein,
R₅₁ is selected from 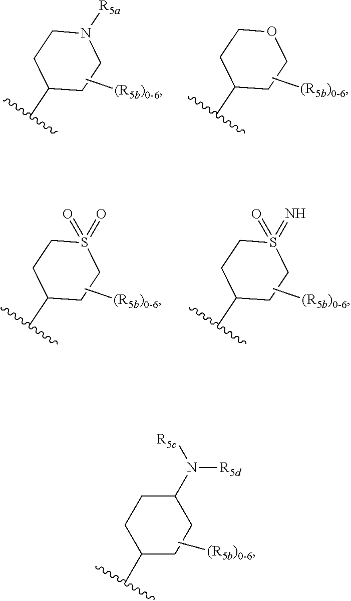
or —C₁₋₆alkyl, said —C₁₋₆alkyl is optionally substituted with 1, 2, 3, 4, 5, or 6 R₅ₑ;
R₅ₐ, R_{5c} and R_{5d} are each independently selected from hydrogen; —C₁₋₆alkyl; or —C₁₋₆alkyl substituted with 1, 2, 3, 4, 5 or 6 substituents selected from —F, —C₁₋₃alkyl, oxo, —OC₁₋₃alkyl, —NH₂, —NHC₁₋₃alkyl, —N(C₁₋₃alkyl)₂, —CN or 3-6 membered cycloalkyl;
R_{5b} and R₅ₑ are each independently selected from —F, —C₁₋₆alkyl, oxo, —OC₁₋₆alkyl, —NH₂, —NHC₁₋₆alkyl, —N(C₁₋₆alkyl)₂, —CN or 1 membered cycloalkyl, wherein, said —C₁₋₆alkyl is optionally substituted with 1, 2, 3 or 4 substituents selected from —F, —C₁₋₃alkyl, oxo, —OC₁₋₃alkyl, —NH₂, —NHC₁₋₃alkyl, —N(C₁₋₃alkyl)₂, —CN or 3-6 membered cycloalkyl;
R₁ is independently selected from —C₁₋₃alkyl; —C₁₋₃haloalkyl; 5 membered heteroaryl containing 1 or 2 heteroatoms selected from N, O or S; or —C₁₋₃alkyl substituted with 1, 2 or 3 substituents selected from —F, —OC₁₋₃alkyl, —NH₂, —NHC₁₋₃alkyl, —N(C₁₋₃alkyl)₂, —CN or 3-6 membered cycloalkyl;
each of R₄ is independently selected from hydrogen, deuterium, —F, —Cl, —C₁₋₃alkyl, —OC₁₋₃alkyl, —NH₂, —NHC₁₋₃alkyl, —N(C₁₋₃alkyl)₂, —CN or 3-6 membered cycloalkyl;
R₁₁ and R₁₂ are independently selected from hydrogen or —C₁₋₃alkyl;
R₁₃ is selected from hydrogen or —C₁₋₃alkyl; or
R₃ is independently selected from phenyl, said phenyl is independently optionally substituted with 1, 2, or 3 substituents selected from R_{3c};
R_{3c} is selected from —F; —C₁₋₃alkyl; —OC₁₋₃alkyl; —NH₂; —NHC₁₋₃alkyl; —N(C₁₋₃alkyl)₂; —C(═O)NH₂, —C(═O)NH(C₁₋₃alkyl); —C(═O)N(C₁₋₃alkyl)₂; —CN; —S(═O)₂NH₂; —S(═O)₂NH(C₁₋₃alkyl); —S(═O)₂N(C₁₋₃alkyl)₂; —S(═O)₂C₁₋₃alkyl; —S(═O)(═NH)CH₃; or —S(═O)(═NCH₃)CH₃.

94. The compound according to claim 93, wherein,
R₅₁ is selected from 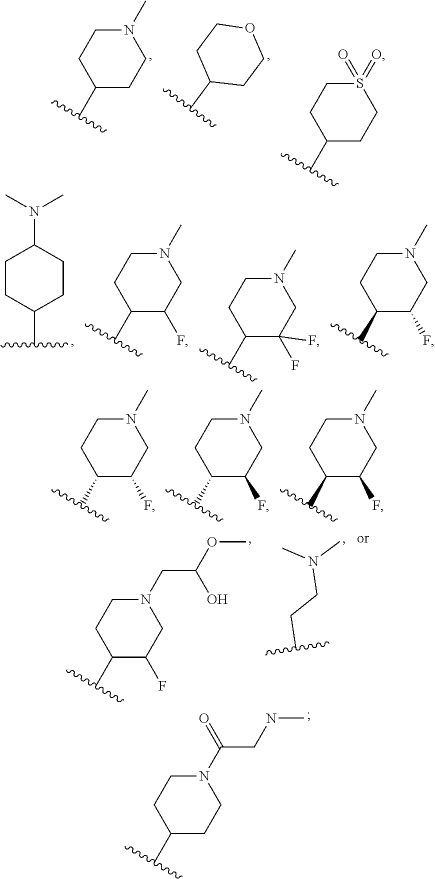
R₁ is independently selected from 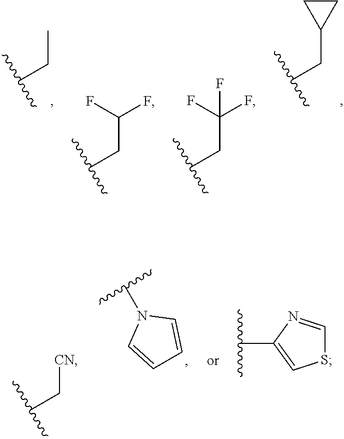
each of R₄ is hydrogen;
R₁₁ and R₁₂ are independently selected from hydrogen;
R₁₃ is selected from hydrogen; or
R₃ is independently selected from

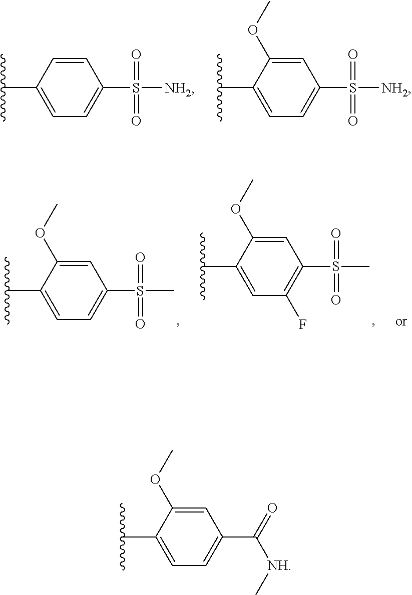

95. The compound according to claim 83, wherein the formula (I) is:

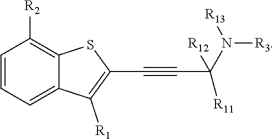

96. The compound according to claim 95, wherein R₁ is independently selected from —C₁₋₃ haloalkyl; or —C₁₋₃alkyl optionally substituted with 1, 2 or 3 substituents selected from —F, —CN, —OC₁₋₃ alkyl, —NH₂, —NHC₁₋₃alkyl, —N(C₁₋₃alkyl)₂ or 3-6 membered cycloalkyl such as cyclopropyl;
R₂ is —NHR₅₁;
R₅₁ is selected from —C₁₋₆alkyl, —C₁₋₆haloalkyl, 3-6 membered cycloalkyl, or 3-10 membered heterocycloalkyl; said —C₁₋₆alkyl, —C₁₋₆haloalkyl, 3-6 membered cycloalkyl, or 3-10 membered heterocycloalkyl are each independently optionally substituted with one or more (such as 1, 2, 3, 4, 5 or 6) substituents selected from halogen, —C₁₋₆alkyl (optionally substituted with one or more of halogen, —CN, oxo, ═NH, —OH, —OC₁₋₆alkyl, or 5-6 membered heterocycloalkyl optionally substituted with one or more of halogen, —CN, oxo, ═NH, —OH, or —OC₁₋₆alkyl), —C₂₋₆alkenyl, —C₂₋₆alkynyl, —C₁₋₆haloalkyl, —CN, —OH, —NH₂, oxo, ═NH, —OC₁₋₆alkyl, —SC₁₋₆alkyl, —C(O)C₁₋₆alkyl, —C(O)C₁₋₆alkyl-NHC₁₋₆alkyl, —C(O)C₁₋₆ alkyl-N(C₁₋₆alkyl)₂, —C(O)NHC₁₋₆alkyl, —C(O)N(C₁₋₆alkyl)₂, —C(O)OC₁₋₆alkyl, —OC(O)C₁₋₆alkyl, —NHC₁₋₆alkyl, —N(C₁₋₆alkyl)₂, 3-6 membered cycloalkyl, or 3-12 membered heterocycloalkyl such as 5-12 membered spiro-heterocyclyl or bridged-heterocyclo (optionally substituted with one or more of halogen, —CN, oxo, ═NH, —OH, or —OC₁₋₆alkyl), wherein said heterocycloalkyl independently contains one or more heteroatoms selected from N, O, or S;
R₃ is independently selected from phenyl, 5-10 membered heterocycloalkenyl containing 1 or 2 heteroatoms selected from N, O or S, or 5-10 membered, preferably 5-6 membered heteroaryl containing 1 or 2 heteroatoms selected from N, O or S; each of which is independently optionally substituted with 1, 2, or 3 substituents selected from R₃ₐ;
wherein R₃ₐ is selected from halogen; oxo; —C₁₋₃alkyl; —OC₁₋₃alkyl; —NH₂; —NHC₁₋₃alkyl; —N(C₁₋₃ alkyl)₂; —C(═O)NH₂, —C(═O)NH(C₁₋₃alkyl); —C(═O)N(C₁₋₃alkyl)₂; —CN; —S(═O)₂NH₂; —S(═O)₂NH(C₁₋₃ alkyl); —S(═O)₂NHC(═O)C₁₋₃alkyl; —S(═O)₂N(C₁₋₃alkyl)₂; —S(═O)₂C₁₋₃alkyl; —S(═O)(═NH)C₁₋₃alkyl; —S(═O)(═NH)C₃₋₆cycloalkyl; —S(═O)(═NR′)C₂₋₆heterocycloalkyl; —S(═O)(═NC₃₋₆cycloalkyl)C₁₋₃alkyl; —S(═O)(═NC₂₋₆heterocycloalkyl)C₁₋₃alkyl; —S(═O)(═NC₁₋₃alkyl)C₁₋₃alkyl; —S(═O)(═NCN)C₁₋₃alkyl; —N(C₁₋₃alkyl)S(═O)₂C₁₋₃alkyl; —PO(C₁₋₃alkyl)₂; 3-6 membered cycloalkyl; 3-6 membered heterocycloalkyl optionally containing 1, 2 or 3 heteroatoms selected from N, O, P or S; wherein said C₁₋₃alkyl, OC₁₋₃alkyl, 3-6 membered cycloalkyl, C₃₋₆cycloalkyl, C₂₋₆heterocycloalkyl or 3-6 membered heterocycloalkyl at each occurrence is independently optionally substituted with 1, 2, 3, 4, 5 or 6 substituents selected from deuterium, halogen, —CN, —C₁₋₃alkyl, oxo, —OH, —OC₁₋₃alkyl, —NH₂, —NHC₁₋₃alkyl, —N(C₁₋₃alkyl)₂, —COOH, —C(O)OC₁₋₃alkyl, —C(═O)NH₂, —C(═O)NH(C₁₋₃alkyl), —C(═O)N(C₁₋₃alkyl)₂, —S(═O)₂NH₂, —S(═O)₂NH(C₁₋₃alkyl), —S(═O)₂C₁₋₃alkyl, —S(═O)₂N(C₁₋₃alkyl)₂, —S(═O)(═NH)C₁₋₃alkyl, —S(═O)(═NC₁₋₃alkyl)C₁₋₃alkyl, or 3-6 membered cycloalkyl.

97. The compound according to claim 95, wherein R₁ is independently selected from 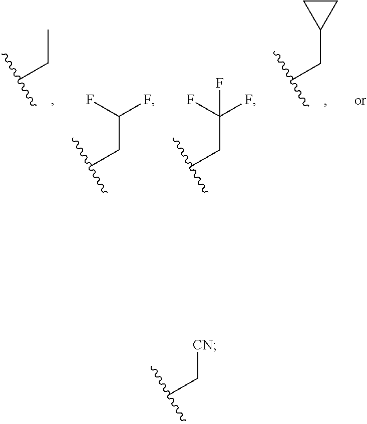
R₅₁ is selected from —C₁₋₆alkyl, 3-6 membered cycloalkyl, or 5-10 membered heterocycloalkyl such as 5-10 membered spiro-heterocyclyl or bridged-heterocyclyl; said —C₁₋₆alkyl, 3-6 membered cycloalkyl, or 5-10 membered heterocycloalkyl are each independently optionally substituted with one or more (such as 1, 2, 3, 4, 5 or 6) substituents selected from halogen, —C₁₋₆alkyl (optionally substituted with one or more of halogen, —CN, oxo, ═NH, —OH, —OC₁₋₆alkyl, or 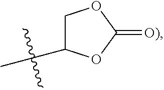
—CN, —OH, —NH₂, oxo, ═NH, —OC₁₋₆alkyl, —SC₁₋₆alkyl, —C(O)C₁₋₆alkyl-NHC₁₋₆alkyl, —C(O)C₁₋₆alkyl-N(C₁₋₆alkyl)₂, —NHC₁₋₆alkyl, —N(C₁₋₆ alkyl)₂, 3-5 membered cycloalkyl, or 5-12 membered heterocycloalkyl such as 5-12 membered spiro-heterocyclyl or bridged-heterocyclyl (optionally substituted with one or more of halogen, —CN, oxo, ═NH, —OH, or —OC₁₋₆alkyl), wherein said heterocycloalkyl independently contains one or more heteroatoms selected from N, O, or S; or
R₃ is independently selected from phenyl, pyridinyl, and 5-10 membered benzoheterocycloalkyl containing 1 or 2 heteroatoms selected from N, O or S, each of which is optionally substituted with 1, 2, or 3 substituents selected from R₃ₐ;
wherein R₃ₐ is selected from deuterium, —F; oxo; —OC₁₋₃alkyl; —C(═O)NH₂, —C(═O)NH(C₁₋₃alkyl); —CN; —S(═O)₂NH₂; —S(═O)₂NH(C₁₋₃alkyl); —S(═O)₂NHC(═O)C₁₋₃alkyl; —S(═O)₂N(C₁₋₃alkyl)₂; —S(═O)₂C₁₋₃alkyl; —S(═O)(═NH)C₁₋₃alkyl; —S(═O)(═NC₁₋₃alkyl)C₁₋₃alkyl; —N(C₁₋₃alkyl)S(═O)₂C₁₋₃alkyl; —PO(C₁₋₃alkyl)₂; 3-6 membered heterocycloalkyl optionally containing 1, 2 or 3 heteroatoms selected from N, O, P or S; wherein said C₁₋₃alkyl, OC₁₋₃alkyl or 3-6 membered heterocycloalkyl at each occurrence is independently optionally substituted with 1, 2, 3, 4, 5 or 6 substituents selected from deuterium, —F, —CN, oxo, —C₁₋₃alkyl, —OH, —OC₁₋₃alkyl, —N(C₁₋₃alkyl)₂, —C(O)OC₁₋₃alkyl, —C(═O)NH₂, —C(═O)NH(C₁₋₃alkyl), or —C(═O)N(C₁₋₃alkyl)₂.

98. The compound according to claim 95, wherein R₅₁ is selected from —C₁₋₃alkyl, cyclohexyl, or 6-8 membered heterocycloalkyl such as 6-8 membered spiro-heterocyclyl or bridged-heterocyclyl; said —C₁₋₃alkyl, cyclohexyl, or 6-8 membered heterocycloalkyl are each independently optionally substituted with one or more (such as 1, 2, 3, 4, 5 or 6) substituents selected from halogen, —C₁₋₆alkyl (optionally substituted with one or more of oxo, ═NH, —OH, —OC₁₋₆alkyl, or 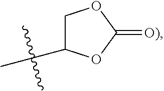
—CN, —OH, NH₂, oxo, ═NH, —OC₁₋₆alkyl, —SC₁₋₆alkyl, —C(O)CH₂—NHC₁₋₆alkyl, —C(O)CH₂—N(C₁₋₆alkyl)₂, —NHC₁₋₆alkyl, —N(C₁₋₆alkyl)₂, cyclopropyl, or 5-12 membered heterocycloalkyl (such as 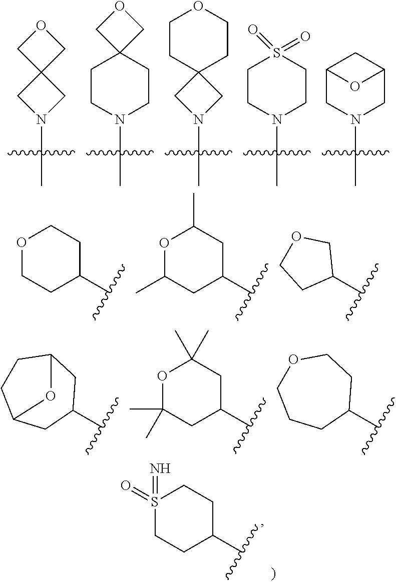
wherein said heterocycloalkyl independently contains one or more heteroatoms selected from N, O, or S; or
R₃ is independently selected from phenyl, pyridinyl, 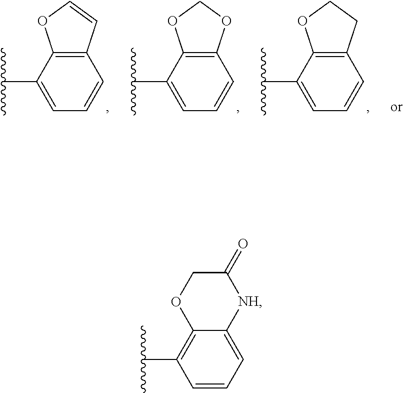
ach of which is optionally substituted with 1, 2, 3 substituents selected from R₃ₐ;
wherein R₃ₐ is selected from —F; oxo; —OC₁₋₃alkyl; —C(═O)NHC₁₋₃alkyl; —S(═O)₂NH₂; —S(═O)₂NHC(═O)CH₃; —S(═O)₂CH₃; —S(═O)(═NH)C₁₋₃alkyl; —S(═O)(═NC₁₋₃alkyl)C₁₋₃alkyl; —N(CH₃)S(═O)₂CH₃; —PO(C₁₋₃alkyl)₂; morpholinyl or 5-6 membered heterocycloalkyl optionally containing 1, 2 or 3 heteroatoms selected from N, 0, or P; wherein said C₁₋₃alkyl, OC₁₋₃alkyl or 5-6 membered heterocycloalkyl at each occurrence is independently optionally substituted with 1, 2, 3, 4, 5 or 6 substituents selected from deuterium, —F, —CN, oxo, —OH, —OCH₃, —N(C₁₋₃alkyl)₂, —C(O)OCH₃, or —C(═O)NH₂.

99. The compound according to claim 95, wherein R₅₁ is 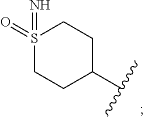 [SMILES: *C1CCS(=N)(=O)CC1]
or
R₃ is independently selected from phenyl optionally substituted with 1, 2, or 3 substituents selected from R₃ₐ;
wherein R₃ₐ is selected from: —F, —OCH₃, —OCD₃, —OCF₃, —OCH₂CN, —OCH₂CF₃, —CH₂F, —CHF₂, —OCH₂CH₂OH, —OCH₂CH₂OCH₃, —S(O)₂NH₂, —S(O)₂NHCOCH₃, —S(O)₂CH₃, —S(═O)(═NH)CH₃, —S(═O)(═NCH₃)CH₃, —N(CH₃)S(═O)₂CH₃, —C(O)NHCH₃, —C(O)NHCH(COOCH₃)CH₂CH₂CONH₂, —PO(CH₃)₂, morpholinyl, or 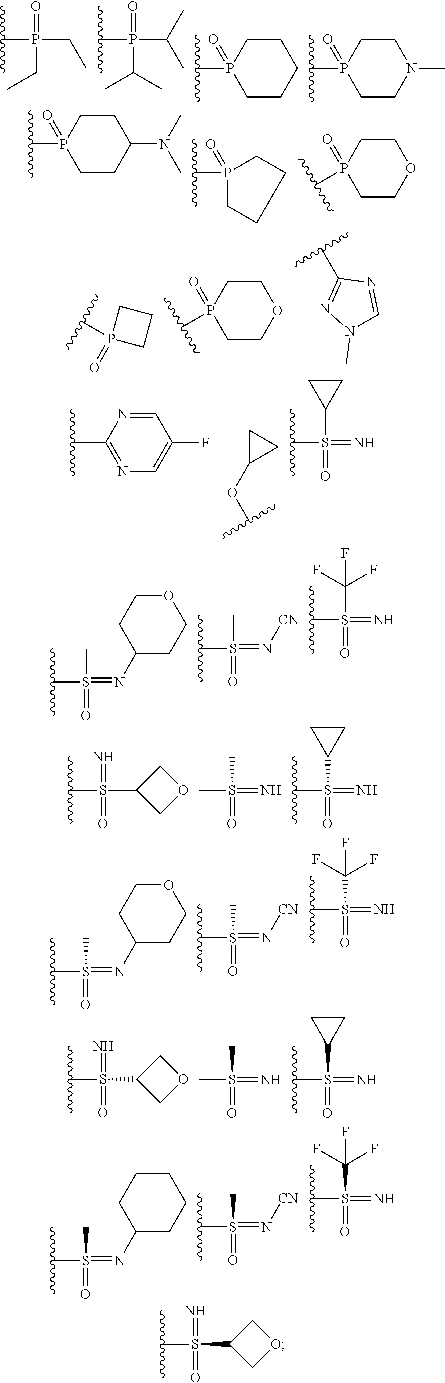
both of R₁₁ and R₁₂ are hydrogen; or
R₁₃ is H.

100. The compound according to claim 95, wherein R₂ is selected from:

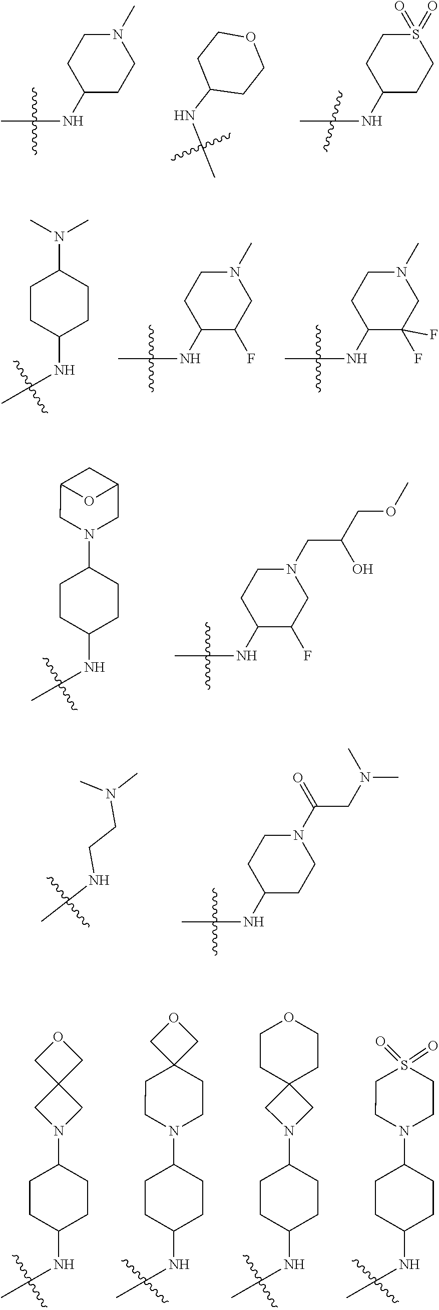

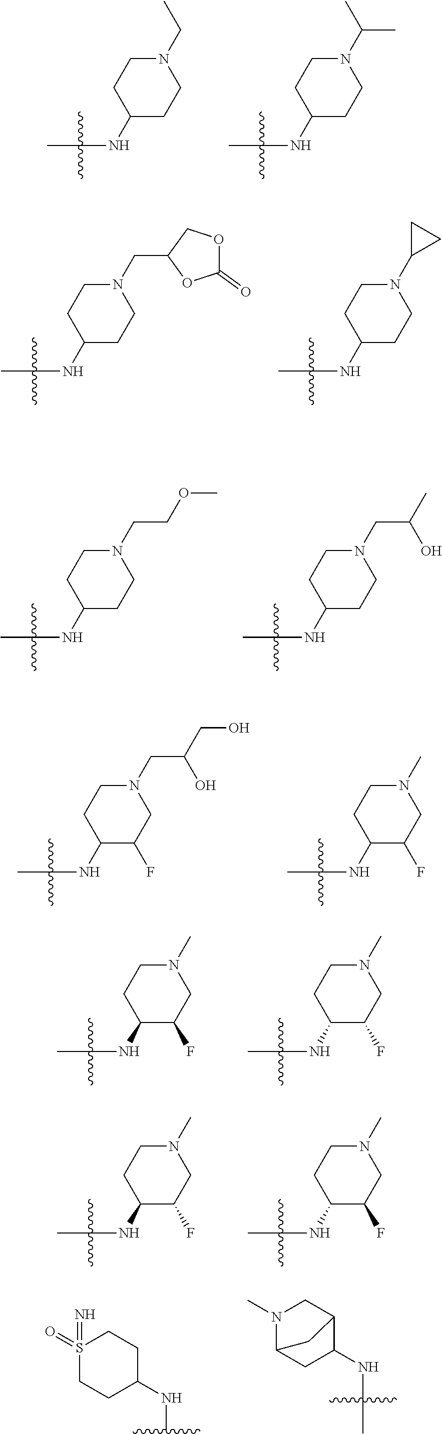

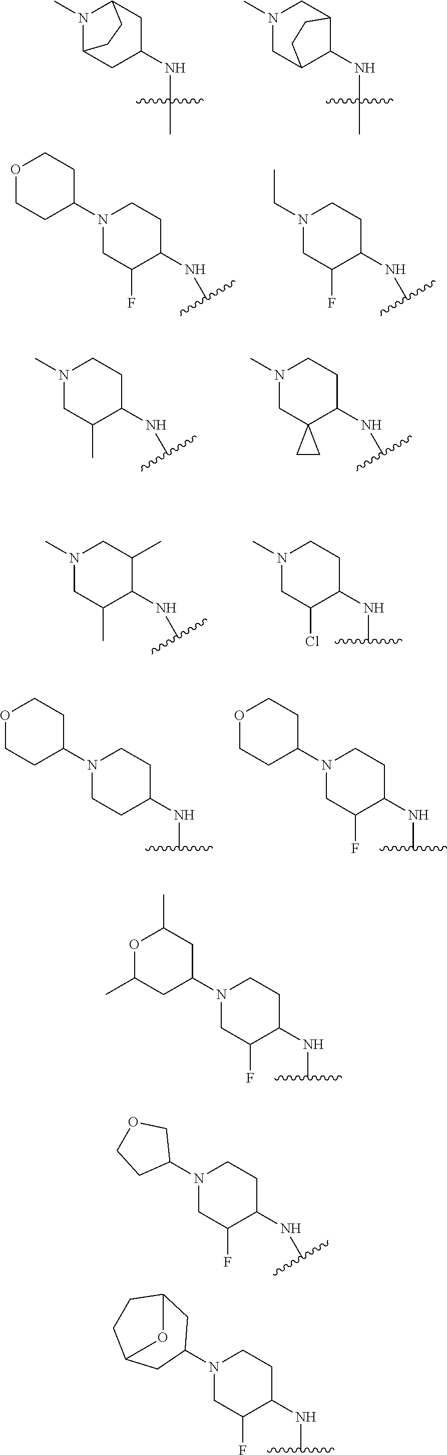

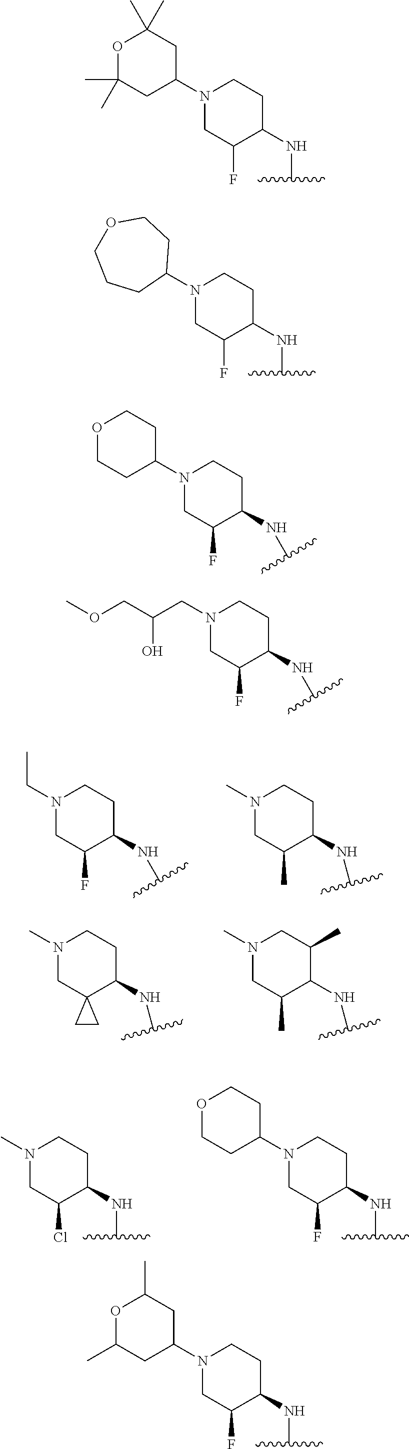

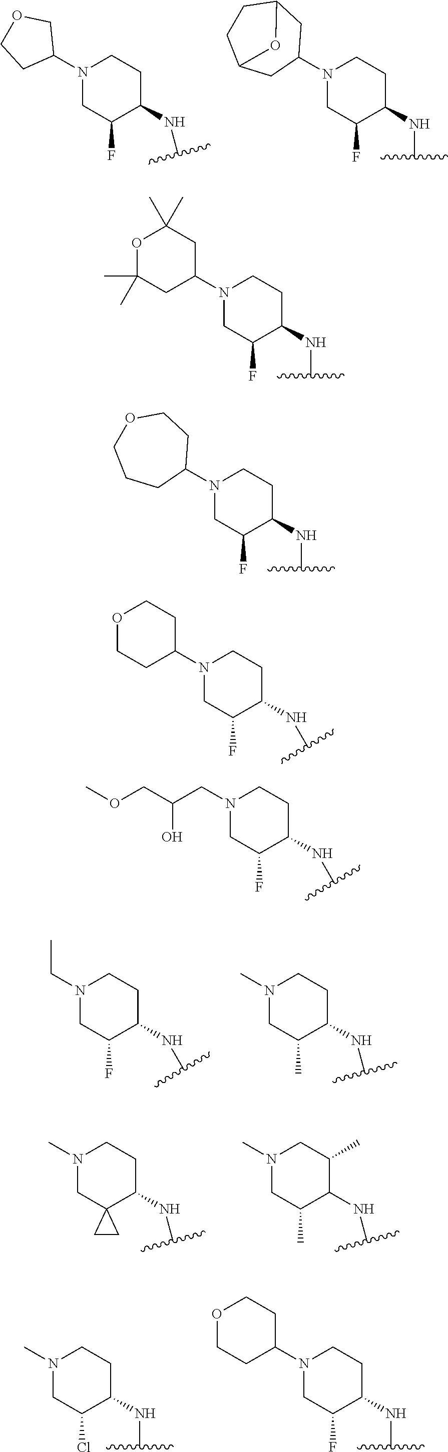

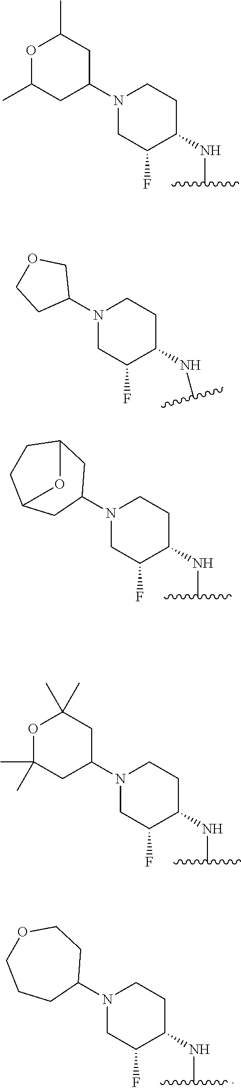 [SMILES: *N[C@H]1CCN(C2CC(C)OC(C)C2)C[C@H]1F ; *N[C@H]1CCN(C2CCOC2)C[C@H]1F ; *N[C@H]1CCN(C2CC3CCC(C2)O3)C[C@H]1F ; *N[C@H]1CCN(C2CC(C)(C)OC(C)(C)C2)C[C@H]1F ; *N[C@H]1CCN(C2CCCOCC2)C[C@H]1F]

101. The compound according to claim 83, wherein the chemical moiety 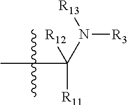
is selected from: 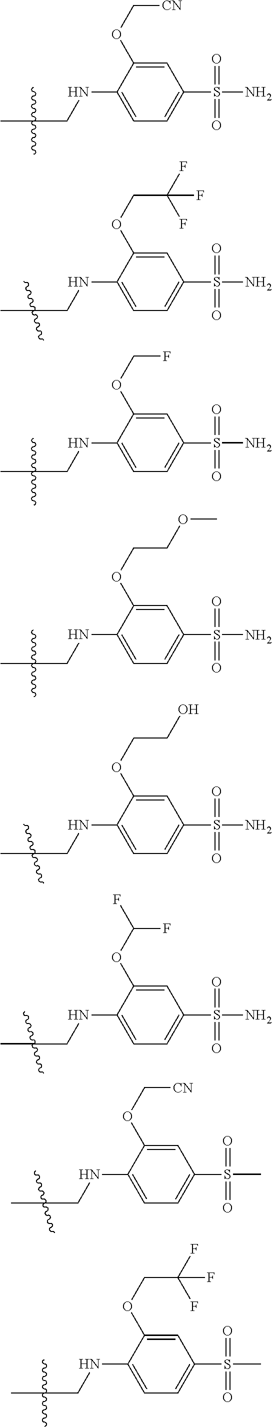 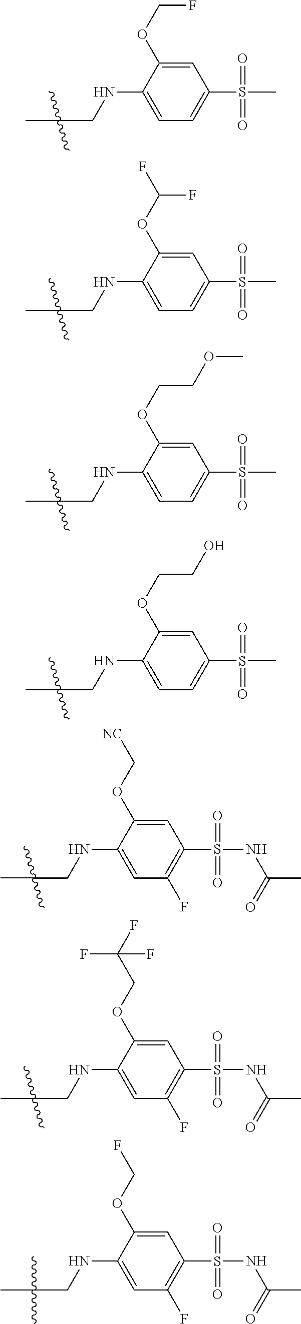 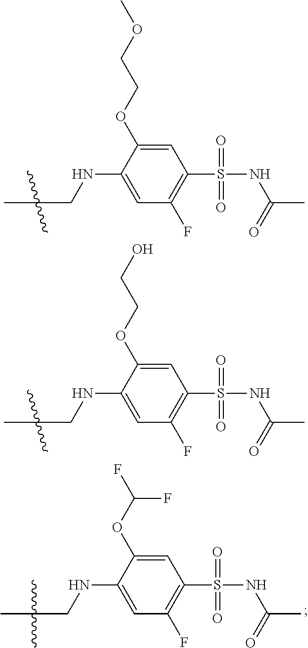
or the chemical moiety 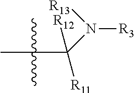
is selected from: 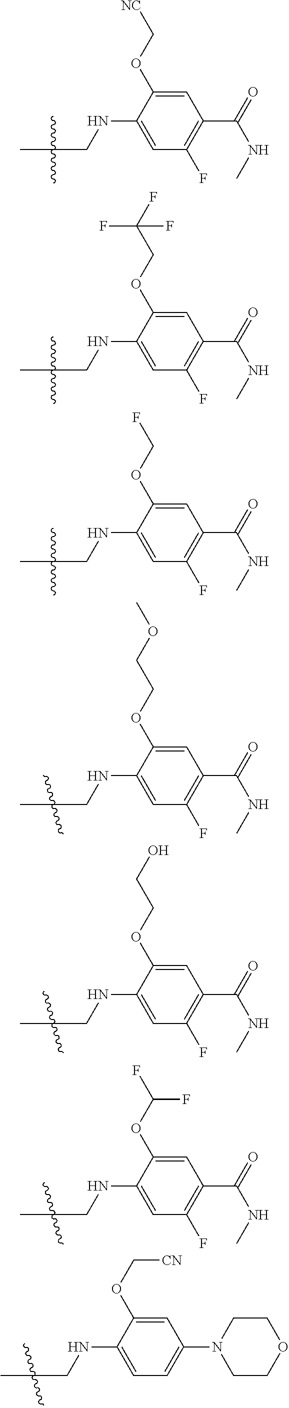 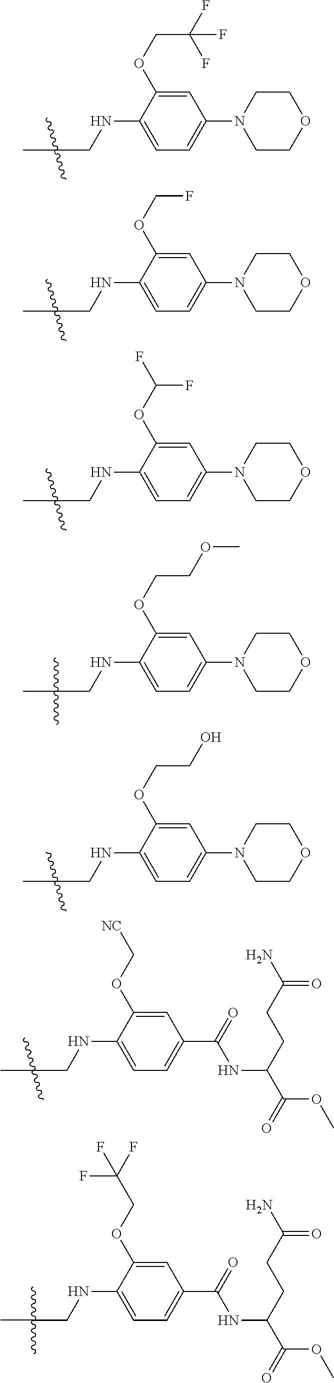 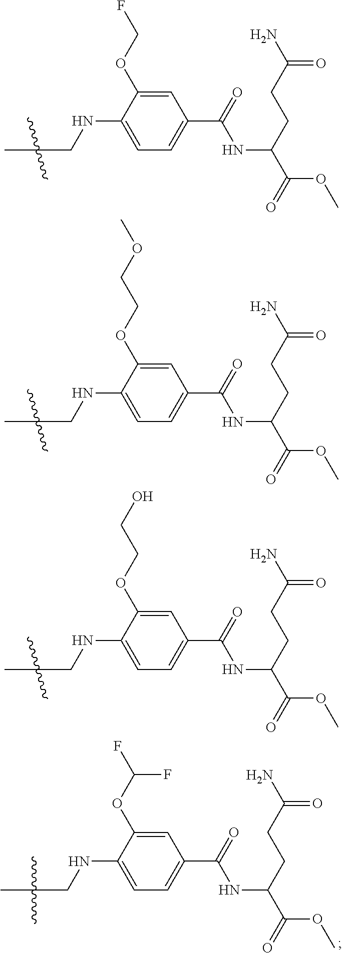 [SMILES: *CNc1ccc(C(=O)NC(CCC(N)=O)C(=O)OC)cc1OCF ; *CNc1ccc(C(=O)NC(CCC(N)=O)C(=O)OC)cc1OCCOC ; *CNc1ccc(C(=O)NC(CCC(N)=O)C(=O)OC)cc1OCCO ; *CNc1ccc(C(=O)NC(CCC(N)=O)C(=O)OC)cc1OC(F)F]
or the chemical moiety 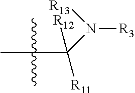
is selected from: 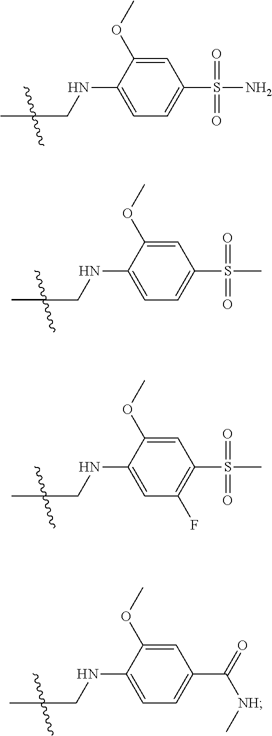 [SMILES: *CNc1ccc(S(N)(=O)=O)cc1OC ; *CNc1ccc(S(C)(=O)=O)cc1OC ; *CNc1cc(F)c(S(C)(=O)=O)cc1OC ; *CNc1ccc(C(=O)NC)cc1OC]
or the chemical moiety 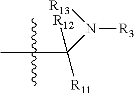
is selected from: 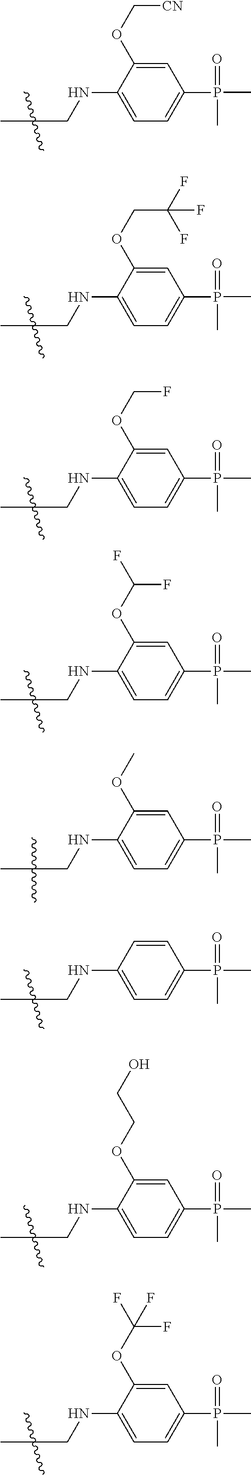 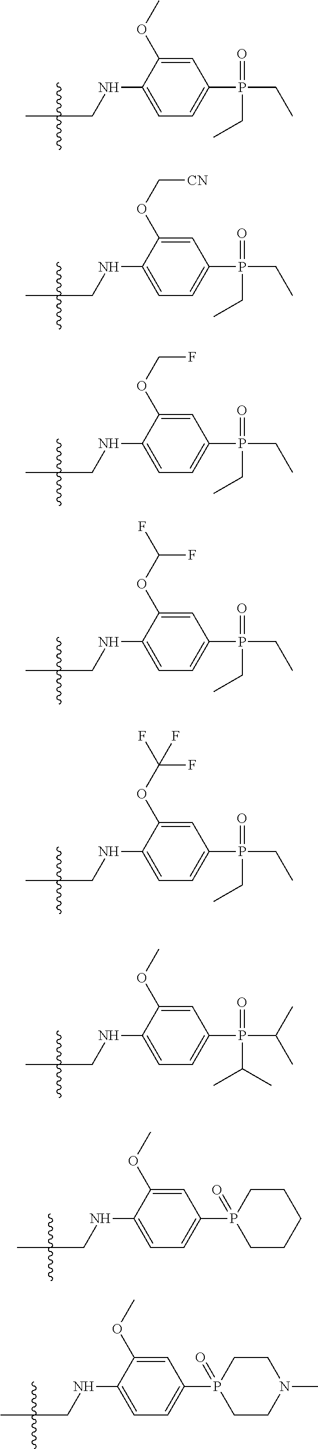 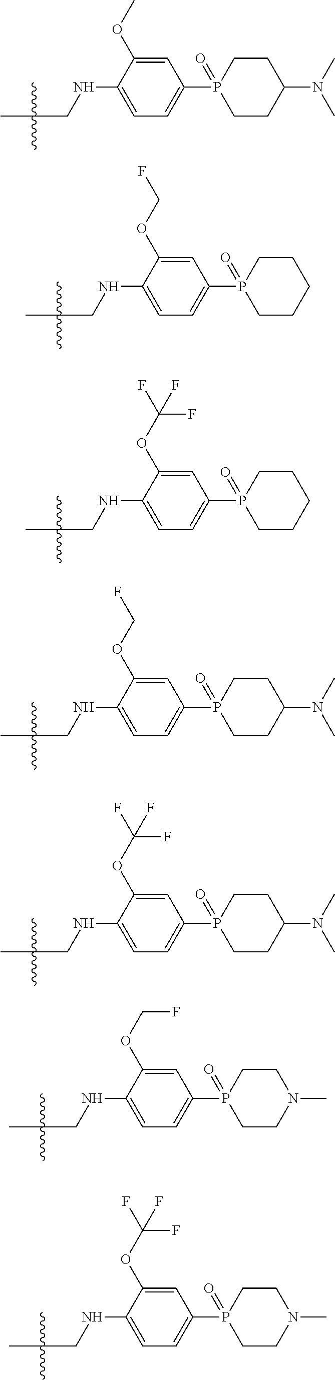 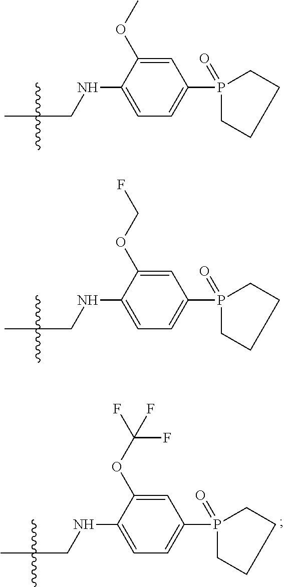 [SMILES: *CNc1ccc(P2(=O)CCCC2)cc1OC ; *CNc1ccc(P2(=O)CCCC2)cc1OCF ; *CNc1ccc(P2(=O)CCCC2)cc1OC(F)(F)F]
or the chemical moiety 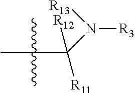
is selected from:

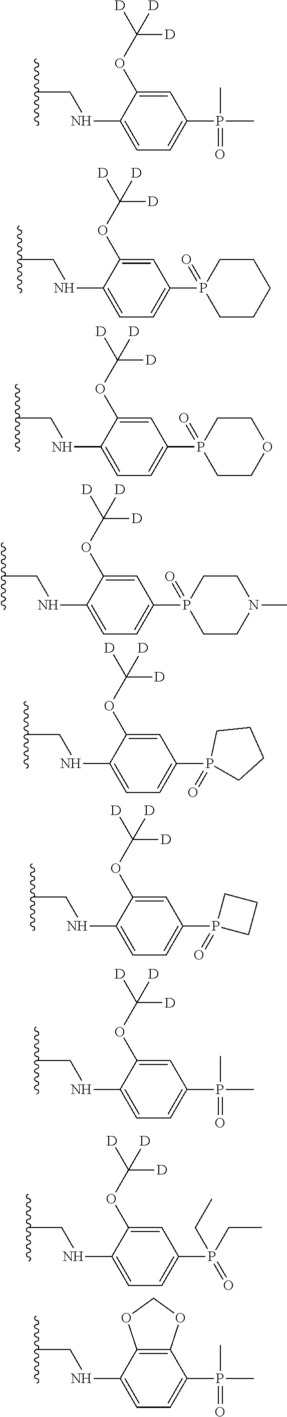

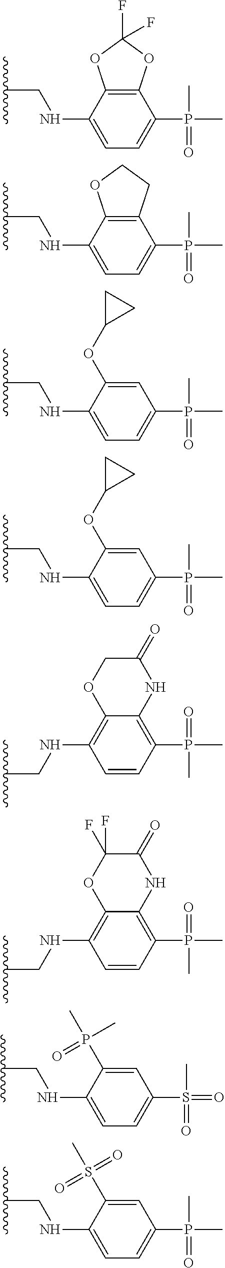

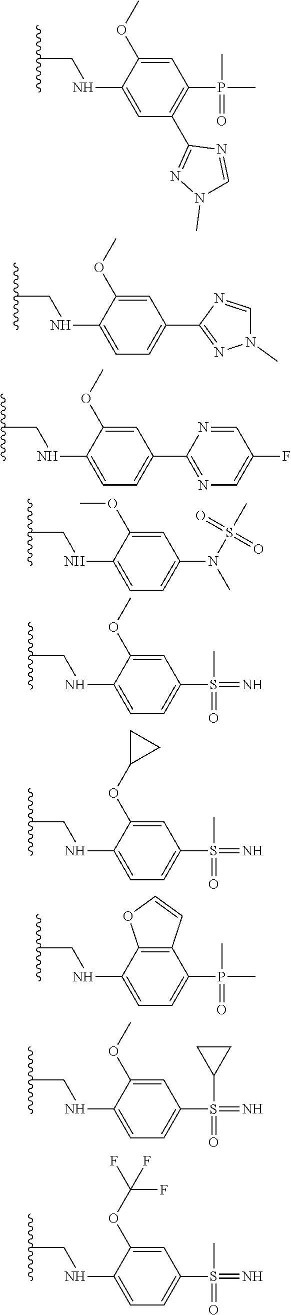

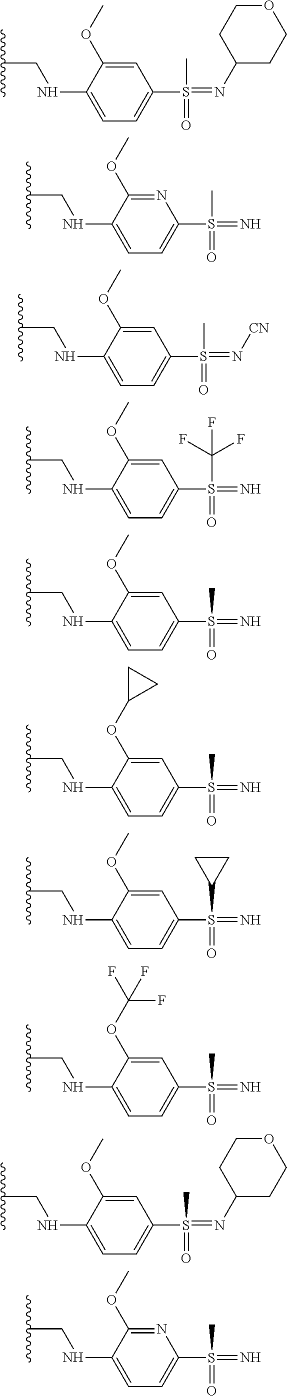

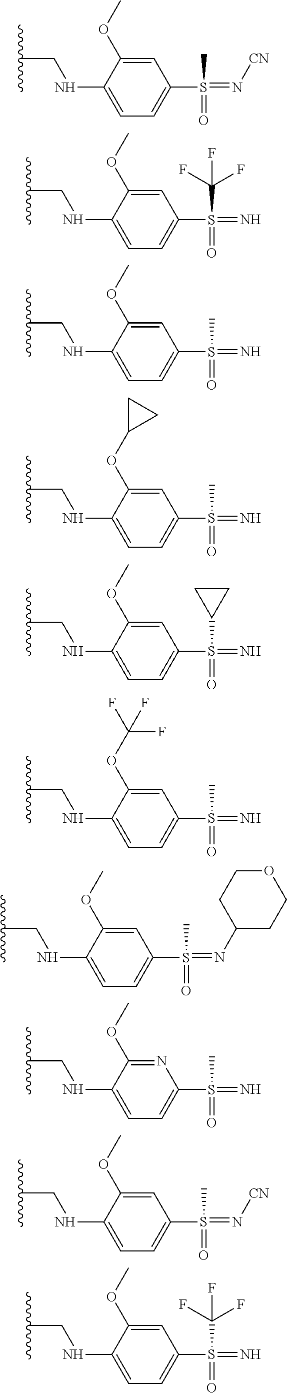

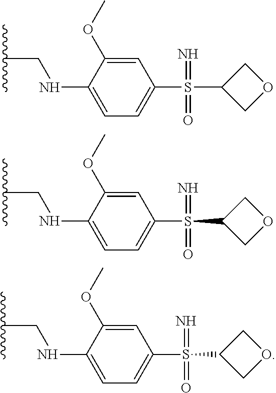

102. The compound according to claim 83, wherein the compound of formula (I) is selected from:

| No. | IUPAC Name |
|---|---|
| 1 | 4-((3-(3-(cyanomethyl)-7-((3,3-difluoro-1-methylpiperidin-4-yl)amino)benzo[b]thiophen-2-yl)prop-2-yn-1- |
| | yl)amino)-3-methoxybenzenesulfonamide; |
| 2 | 4-((3-(7-((3,3-difluoro-1-methylpiperidin-4-yl)amino)-3-(thiazol-4-yl)benzo[b]thiophen-2-yl)prop-2-yn-1- |
| | yl)amino)-3-methoxybenzenesulfonamide; |
| 3 | 4-((3-(7-((3,3-difluoro-1-methylpiperidin-4-yl)amino)-3-(2,2,2-trifluoroethyl)benzo[b]thiophen-2-yl)prop- |
| | 2-yn-1-yl)amino)-3-methoxybenzenesulfonamide; |
| 4 | 4-((3-(3-(cyclopropylmethyl)-7-((3,3-difluoro-1-methylpiperidin-4-yl)amino)benzo[b]thiophen-2-yl)prop-2- |
| | yn-1-yl)amino)-3-methoxybenzenesulfonamide; |
| 5 | 4-((3-(7-((3,3-difluoro-1-methylpiperidin-4-yl)amino)-3-(2,2-difluoroethyl)benzo[b]thiophen-2-yl)prop-2- |
| | yn-1-yl)amino)-3-methoxybenzenesulfonamide; |
| 6 | 4-((3-(7-((3,3-difluoro-1-methylpiperidin-4-yl)amino)-3-(1H-pyrrol-1-yl)benzo[b]thiophen-2-yl)prop-2-yn- |
| | 1-yl)amino)-3-methoxybenzenesulfonamide; |
| 7 | 4-((3-(7-((3,3-difluoro-1-methylpiperidin-4-yl)amino)-3-ethylbenzo[b]thiophen-2-yl)prop-2-yn-1- |
| | yl)amino)-3-methoxybenzenesulfonamide; |
| 8 | 4-((3-(3-(cyanomethyl)-7-((1,1-dioxidotetrahydro-2H-thiopyran-4-yl)amino)benzo[b]thiophen-2-yl)prop-2- |
| | yn-1-yl)amino)-3-methoxybenzenesulfonamide; |
| 9 | 4-((3-(7-((1,1-dioxidotetrahydro-2H-thiopyran-4-yl)amino)-3-(thiazol-4-yl)benzo[b]thiophen-2-yl)prop-2- |
| | yn-1-yl)amino)-3-methoxybenzenesulfonamide; |
| 10 | 4-((3-(7-((1,1-dioxidotetrahydro-2H-thiopyran-4-yl)amino)-3-(2,2,2-trifluoroethyl)benzo[b]thiophen-2- |
| | yl)prop-2-yn-1-yl)amino)-3-methoxybenzenesulfonamide; |
| 11 | 4-((3-(3-(cyclopropylmethyl)-7-((1,1-dioxidotetrahydro-2H-thiopyran-4-yl)amino)benzo[b]thiophen-2- |
| | yl)prop-2-yn-1-yl)amino)-3-methoxybenzenesulfonamide; |
| 12 | 4-((3-(3-(2,2-difluoroethyl)-7-((1,1-dioxidotetrahydro-2H-thiopyran-4-yl)amino)benzo[b]thiophen-2- |
| | yl)prop-2-yn-1-yl)amino)-3-methoxybenzenesulfonamide; |
| 13 | 4-((3-(7-((1,1-dioxidotetrahydro-2H-thiopyran-4-yl)amino)-3-(1H-pyrrol-1-yl)benzo[b]thiophen-2-yl)prop- |
| | 2-yn-1-yl)amino)-3-methoxybenzenesulfonamide; |
| 14 | 4-((3-(7-((1,1-dioxidotetrahydro-2H-thiopyran-4-yl)amino)-3-ethylbenzo[b]thiophen-2-yl)prop-2-yn-1- |
| | yl)amino)-3-methoxybenzenesulfonamide; |
| 15 | 4-((3-(3-(cyanomethyl)-7-(((3R,4S)-3-fluoro-1-methylpiperidin-4-yl)amino)benzo[b]thiophen-2-yl)prop-2- |
| | yn-1-yl)amino)-3-methoxybenzenesulfonamide; |
| 16 | 4-((3-(7-(((3R,4S)-3-fluoro-1-methylpiperidin-4-yl)amino)-3-(thiazol-4-yl)benzo[b]thiophen-2-yl)prop-2- |
| | yn-1-yl)amino)-3-methoxybenzenesulfonamide; |
| 17 | 4-((3-(7-(((3R,4S)-3-fluoro-1-methylpiperidin-4-yl)amino)-3-(2,2,2-trifluoroethyl)benzo[b]thiophen-2- |
| | yl)prop-2-yn-1-yl)amino)-3-methoxybenzenesulfonamide; |
| 18 | 4-((3-(3-(cyclopropylmethyl)-7-(((3R,4S)-3-fluoro-1-methylpiperidin-4-yl)amino)benzo[b]thiophen-2- |
| | yl)prop-2-yn-1-yl)amino)-3-methoxybenzenesulfonamide; |
| 19 | 4-((3-(3-(2,2-difluoroethyl)-7-(((3R,4S)-3-fluoro-1-methylpiperidin-4-yl)amino)benzo[b]thiophen-2- |
| | yl)prop-2-yn-1-yl)amino)-3-methoxybenzenesulfonamide; |
| 20 | 4-((3-(7-(((3R,4S)-3-fluoro-1-methylpiperidin-4-yl)amino)-3-(1H-pyrrol-1-yl)benzo[b]thiophen-2-yl)prop- |
| | 2-yn-1-yl)amino)-3-methoxybenzenesulfonamide; |
| 21 | 4-((3-(3-ethyl-7-(((3R,4S)-3-fluoro-1-methylpiperidin-4-yl)amino)benzo[b]thiophen-2-yl)prop-2-yn-1- |
| | yl)amino)-3-methoxybenzenesulfonamide; |
| 22 | 4-((3-(3-(cyanomethyl)-7-((3-fluoro-1-methylpiperidin-4-yl)amino)benzo[b]thiophen-2-yl)prop-2-yn-1- |
| | yl)amino)-3-methoxybenzenesulfonamide; |
| 23 | 4-((3-(7-((3-fluoro-1-methylpiperidin-4-yl)amino)-3-(thiazol-4-yl)benzo[b]thiophen-2-yl)prop-2-yn-1- |
| | yl)amino)-3-methoxybenzenesulfonamide; |
| 24 | 4-((3-(7-((3-fluoro-1-methylpiperidin-4-yl)amino)-3-(2,2,2-trifluoroethyl)benzo[b]thiophen-2-yl)prop-2-yn- |
| | 1-yl)amino)-3-methoxybenzenesulfonamide; |
| 25 | 4-((3-(3-(cyclopropylmethyl)-7-((3-fluoro-1-methylpiperidin-4-yl)amino)benzo[b]thiophen-2-yl)prop-2-yn- |
| | 1-yl)amino)-3-methoxybenzenesulfonamide; |
| 26 | 4-((3-(3-(2,2-difluoroethyl)-7-((3-fluoro-1-methylpiperidin-4-yl)amino)benzo[b]thiophen-2-yl)prop-2-yn-1- |
| | yl)amino)-3-methoxybenzenesulfonamide; |
| 27 | 4-((3-(7-((3-fluoro-1-methylpiperidin-4-yl)amino)-3-(1H-pyrrol-1-yl)benzo[b]thiophen-2-yl)prop-2-yn-1- |
| | yl)amino)-3-methoxybenzenesulfonamide; |
| 28 | 4-((3-(3-ethyl-7-((3-fluoro-1-methylpiperidin-4-yl)amino)benzo[b]thiophen-2-yl)prop-2-yn-1-yl)amino)-3- |
| | methoxybenzenesulfonamide; |
| 29 | 4-((3-(3-(cyanomethyl)-7-((1-(dimethylglycyl)piperidin-4-yl)amino)benzo[b]thiophen-2-yl)prop-2-yn-1- |
| | yl)amino)-3-methoxybenzenesulfonamide; |
| 30 | 4-((3-(7-((1-(dimethylglycyl)piperidin-4-yl)amino)-3-(thiazol-4-yl)benzo[b]thiophen-2-yl)prop-2-yn-1- |
| | yl)amino)-3-methoxybenzenesulfonamide; |
| 31 | 4-((3-(7-((1-(dimethylglycyl)piperidin-4-yl)amino)-3-(2,2,2-trifluoroethyl)benzo[b]thiophen-2-yl)prop-2- |
| | yn-1-yl)amino)-3-methoxybenzenesulfonamide; |
| 32 | 4-((3-(3-(cyclopropylmethyl)-7-((1-(dimethylglycyl)piperidin-4-yl)amino)benzo[b]thiophen-2-yl)prop-2- |
| | yn-1-yl)amino)-3-methoxybenzenesulfonamide; |
| 33 | 4-((3-(3-(2,2-difluoroethyl)-7-((1-(dimethylglycyl)piperidin-4-yl)amino)benzo[b]thiophen-2-yl)prop-2-yn- |
| | 1-yl)amino)-3-methoxybenzenesulfonamide; |
| 34 | 4-((3-(7-((1-(dimethylglycyl)piperidin-4-yl)amino)-3-(1H-pyrrol-1-yl)benzo[b]thiophen-2-yl)prop-2-yn-1- |
| | yl)amino)-3-methoxybenzenesulfonamide; |
| 35 | 4-((3-(7-((1-(dimethylglycyl)piperidin-4-yl)amino)-3-ethylbenzo[b]thiophen-2-yl)prop-2-yn-1-yl)amino)-3- |
| | methoxybenzenesulfonamide; |
| 36 | 4-((3-(3-(cyanomethyl)-7-((3-fluoro-1-(2-hydroxy-3-methoxypropyl)piperidin-4- |
| | yl)amino)benzo[b]thiophen-2-yl)prop-2-yn-1-yl)amino)-3-methoxybenzenesulfonamide; |
| 37 | 4-((3-(7-((3-fluoro-1-(2-hydroxy-3-methoxypropyl)piperidin-4-yl)amino)-3-(thiazol-4-yl)benzo[b]thiophen- |
| | 2-yl)prop-2-yn-1-yl)amino)-3-methoxybenzenesulfonamide; |
| 38 | 4-((3-(7-((3-fluoro-1-(2-hydroxy-3-methoxypropyl)piperidin-4-yl)amino)-3-(2,2,2- |
| | trifluoroethyl)benzo[b]thiophen-2-yl)prop-2-yn-1-yl)amino)-3-methoxybenzenesulfonamide; |
| 39 | 4-((3-(3-(cyclopropylmethyl)-7-((3-fluoro-1-(2-hydroxy-3-methoxypropyl)piperidin-4- |
| | yl)amino)benzo[b]thiophen-2-yl)prop-2-yn-1-yl)amino)-3-methoxybenzenesulfonamide; |
| 40 | 4-((3-(3-(2,2-difluoroethyl)-7-((3-fluoro-1-(2-hydroxy-3-methoxypropyl)piperidin-4- |
| | yl)amino)benzo[b]thiophen-2-yl)prop-2-yn-1-yl)amino)-3-methoxybenzenesulfonamide; |
| 41 | 4-((3-(7-((3-fluoro-1-(2-hydroxy-3-methoxypropyl)piperidin-4-yl)amino)-3-(1H-pyrrol-1- |
| | yl)benzo[b]thiophen-2-yl)prop-2-yn-1-yl)amino)-3-methoxybenzenesulfonamide; |
| 42 | 4-((3-(3-ethyl-7-((3-fluoro-1-(2-hydroxy-3-methoxypropyl)piperidin-4-yl)amino)benzo[b]thiophen-2- |
| | yl)prop-2-yn-1-yl)amino)-3-methoxybenzenesulfonamide; |
| 43 | 4-((3-(3-(cyanomethyl)-7-(((3R,4R)-3-fluoro-1-methylpiperidin-4-yl)amino)benzo[b]thiophen-2-yl)prop-2- |
| | yn-1-yl)amino)-3-methoxybenzenesulfonamide; |
| 44 | 4-((3-(7-(((3R,4R)-3-fluoro-1-methylpiperidin-4-yl)amino)-3-(thiazol-4-yl)benzo[b]thiophen-2-yl)prop-2- |
| | yn-1-yl)amino)-3-methoxybenzenesulfonamide; |
| 45 | 4-((3-(7-(((3R,4R)-3-fluoro-1-methylpiperidin-4-yl)amino)-3-(2,2,2-trifluoroethyl)benzo[b]thiophen-2- |
| | yl)prop-2-yn-1-yl)amino)-3-methoxybenzenesulfonamide; |
| 46 | 4-((3-(3-(cyclopropylmethyl)-7-(((3R,4R)-3-fluoro-1-methylpiperidin-4-yl)amino)benzo[b]thiophen-2- |
| | yl)prop-2-yn-1-yl)amino)-3-methoxybenzenesulfonamide; |
| 47 | 4-((3-(3-(2,2-difluoroethyl)-7-(((3R,4R)-3-fluoro-1-methylpiperidin-4-yl)amino)benzo[b]thiophen-2- |
| | yl)prop-2-yn-1-yl)amino)-3-methoxybenzenesulfonamide; |
| 48 | 4-((3-(7-(((3R,4R)-3-fluoro-1-methylpiperidin-4-yl)amino)-3-(1H-pyrrol-1-yl)benzo[b]thiophen-2-yl)prop- |
| | 2-yn-1-yl)amino)-3-methoxybenzenesulfonamide; |
| 49 | 4-((3-(3-ethyl-7-(((3R,4R)-3-fluoro-1-methylpiperidin-4-yl)amino)benzo[b]thiophen-2-yl)prop-2-yn-1- |
| | yl)amino)-3-methoxybenzenesulfonamide; |
| 50 | 4-((3-(3-(cyanomethyl)-7-(((3S,4S)-3-fluoro-1-methylpiperidin-4-yl)amino)benzo[b]thiophen-2-yl)prop-2- |
| | yn-1-yl)amino)-3-methoxybenzenesulfonamide; |
| 51 | 4-((3-(7-(((3S,4S)-3-fluoro-1-methylpiperidin-4-yl)amino)-3-(thiazol-4-yl)benzo[b]thiophen-2-yl)prop-2- |
| | yn-1-yl)amino)-3-methoxybenzenesulfonamide; |
| 52 | 4-((3-(7-(((3S,4S)-3-fluoro-1-methylpiperidin-4-yl)amino)-3-(2,2,2-trifluoroethyl)benzo[b]thiophen-2- |
| | yl)prop-2-yn-1-yl)amino)-3-methoxybenzenesulfonamide; |
| 53 | 4-((3-(3-(cyclopropylmethyl)-7-(((3S,4S)-3-fluoro-1-methylpiperidin-4-yl)amino)benzo[b]thiophen-2- |
| | yl)prop-2-yn-1-yl)amino)-3-methoxybenzenesulfonamide; |
| 54 | 4-((3-(3-(2,2-difluoroethyl)-7-(((3S,4S)-3-fluoro-1-methylpiperidin-4-yl)amino)benzo[b]thiophen-2- |
| | yl)prop-2-yn-1-yl)amino)-3-methoxybenzenesulfonamide; |
| 55 | 4-((3-(7-(((3S,4S)-3-fluoro-1-methylpiperidin-4-yl)amino)-3-(1H-pyrrol-1-yl)benzo[b]thiophen-2-yl)prop- |
| | 2-yn-1-yl)amino)-3-methoxybenzenesulfonamide; |
| 56 | 4-((3-(3-ethyl-7-(((3S,4S)-3-fluoro-1-methylpiperidin-4-yl)amino)benzo[b]thiophen-2-yl)prop-2-yn-1- |
| | yl)amino)-3-methoxybenzenesulfonamide; |
| 57 | 4-((3-(3-(cyanomethyl)-7-((4-(dimethylamino)cyclohexyl)amino)benzo[b]thiophen-2-yl)prop-2-yn-1- |
| | yl)amino)-3-methoxybenzenesulfonamide; |
| 58 | 4-((3-(7-((4-(dimethylamino)cyclohexyl)amino)-3-(thiazol-4-yl)benzo[b]thiophen-2-yl)prop-2-yn-1- |
| | yl)amino)-3-methoxybenzenesulfonamide; |
| 59 | 4-((3-(7-((4-(dimethylamino)cyclohexyl)amino)-3-(2,2,2-trifluoroethyl)benzo[b]thiophen-2-yl)prop-2-yn-1- |
| | yl)amino)-3-methoxybenzenesulfonamide; |
| 60 | 4-((3-(3-(cyclopropylmethyl)-7-((4-(dimethylamino)cyclohexyl)amino)benzo[b]thiophen-2-yl)prop-2-yn-1- |
| | yl)amino)-3-methoxybenzenesulfonamide; |
| 61 | 4-((3-(3-(2,2-difluoroethyl)-7-((4-(dimethylamino)cyclohexyl)amino)benzo[b]thiophen-2-yl)prop-2-yn-1- |
| | yl)amino)-3-methoxybenzenesulfonamide; |
| 62 | 4-((3-(7-((4-(dimethylamino)cyclohexyl)amino)-3-(1H-pyrrol-1-yl)benzo[b]thiophen-2-yl)prop-2-yn-1- |
| | yl)amino)-3-methoxybenzenesulfonamide; |
| 63 | 4-((3-(7-((4-(dimethylamino)cyclohexyl)amino)-3-ethylbenzo[b]thiophen-2-yl)prop-2-yn-1-yl)amino)-3- |
| | methoxybenzenesulfonamide; |
| 64 | 4-((3-(3-(cyanomethyl)-7-((2-(dimethylamino)ethyl)amino)benzo[b]thiophen-2-yl)prop-2-yn-1-yl)amino)- |
| | 3-methoxybenzenesulfonamide; |
| 65 | 4-((3-(7-((2-(dimethylamino)ethyl)amino)-3-(thiazol-4-yl)benzo[b]thiophen-2-yl)prop-2-yn-1-yl)amino)-3- |
| | methoxybenzenesulfonamide; |
| 66 | 4-((3-(7-((2-(dimethylamino)ethyl)amino)-3-(2,2,2-trifluoroethyl)benzo[b]thiophen-2-yl)prop-2-yn-1- |
| | yl)amino)-3-methoxybenzenesulfonamide; |
| 67 | 4-((3-(3-(cyclopropylmethyl)-7-((2-(dimethylamino)ethyl)amino)benzo[b]thiophen-2-yl)prop-2-yn-1- |
| | yl)amino)-3-methoxybenzenesulfonamide; |
| 68 | 4-((3-(3-(2,2-difluoroethyl)-7-((2-(dimethylamino)ethyl)amino)benzo[b]thiophen-2-yl)prop-2-yn-1- |
| | yl)amino)-3-methoxybenzenesulfonamide; |
| 69 | 4-((3-(7-((2-(dimethylamino)ethyl)amino)-3-(1H-pyrrol-1-yl)benzo[b]thiophen-2-yl)prop-2-yn-1- |
| | yl)amino)-3-methoxybenzenesulfonamide; |
| 70 | 4-((3-(7-((2-(dimethylamino)ethyl)amino)-3-ethylbenzo[b]thiophen-2-yl)prop-2-yn-1-yl)amino)-3- |
| | methoxybenzenesulfonamide; |
| 71 | 4-((3-(3-(cyanomethyl)-7-(((3S,4R)-3-fluoro-1-methylpiperidin-4-yl)amino)benzo[b]thiophen-2-yl)prop-2- |
| | yn-1-yl)amino)-3-methoxybenzenesulfonamide; |
| 72 | 4-((3-(7-(((3S,4R)-3-fluoro-1-methylpiperidin-4-yl)amino)-3-(thiazol-4-yl)benzo[b]thiophen-2-yl)prop-2- |
| | yn-1-yl)amino)-3-methoxybenzenesulfonamide; |
| 73 | 4-((3-(7-(((3S,4R)-3-fluoro-1-methylpiperidin-4-yl)amino)-3-(2,2,2-trifluoroethyl)benzo[b]thiophen-2- |
| | yl)prop-2-yn-1-yl)amino)-3-methoxybenzenesulfonamide; |
| 74 | 4-((3-(3-(cyclopropylmethyl)-7-(((3S,4R)-3-fluoro-1-methylpiperidin-4-yl)amino)benzo[b]thiophen-2- |
| | yl)prop-2-yn-1-yl)amino)-3-methoxybenzenesulfonamide; |
| 75 | 4-((3-(3-(2,2-difluoroethyl)-7-(((3S,4R)-3-fluoro-1-methylpiperidin-4-yl)amino)benzo[b]thiophen-2- |
| | yl)prop-2-yn-1-yl)amino)-3-methoxybenzenesulfonamide; |
| 76 | 4-((3-(7-(((3S,4R)-3-fluoro-1-methylpiperidin-4-yl)amino)-3-(1H-pyrrol-1-yl)benzo[b]thiophen-2-yl)prop- |
| | 2-yn-1-yl)amino)-3-methoxybenzenesulfonamide; |
| 77 | 4-((3-(3-ethyl-7-(((3S,4R)-3-fluoro-1-methylpiperidin-4-yl)amino)benzo[b]thiophen-2-yl)prop-2-yn-1- |
| | yl)amino)-3-methoxybenzenesulfonamide; |
| 78 | 4-((3-(3-(cyanomethyl)-7-((1-methylpiperidin-4-yl)amino)benzo[b]thiophen-2-yl)prop-2-yn-1-yl)amino)-3- |
| | methoxybenzenesulfonamide; |
| 79 | 3-methoxy-4-((3-(7-((1-methylpiperidin-4-yl)amino)-3-(thiazol-4-yl)benzo[b]thiophen-2-yl)prop-2-yn-1- |
| | yl)amino)benzenesulfonamide; |
| 80 | 3-methoxy-4-((3-(7-((1-methylpiperidin-4-yl)amino)-3-(2,2,2-trifluoroethyl)benzo[b]thiophen-2-yl)prop-2- |
| | yn-1-yl)amino)benzenesulfonamide; |
| 81 | 4-((3-(3-(cyclopropylmethyl)-7-((1-methylpiperidin-4-yl)amino)benzo[b]thiophen-2-yl)prop-2-yn-1- |
| | yl)amino)-3-methoxybenzenesulfonamide; |
| 82 | 4-((3-(3-(2,2-difluoroethyl)-7-((1-methylpiperidin-4-yl)amino)benzo[b]thiophen-2-yl)prop-2-yn-1- |
| | yl)amino)-3-methoxybenzenesulfonamide; |
| 83 | 3-methoxy-4-((3-(7-((1-methylpiperidin-4-yl)amino)-3-(1H-pyrrol-1-yl)benzo[b]thiophen-2-yl)prop-2-yn- |
| | 1-yl)amino)benzenesulfonamide; |
| 84 | 4-((3-(3-ethyl-7-((1-methylpiperidin-4-yl)amino)benzo[b]thiophen-2-yl)prop-2-yn-1-yl)amino)-3- |
| | methoxybenzenesulfonamide; |
| 85 | 4-((3-(3-(cyanomethyl)-7-((tetrahydro-2H-pyran-4-yl)amino)benzo[b]thiophen-2-yl)prop-2-yn-1-yl)amino)- |
| | 3-methoxybenzenesulfonamide; |
| 86 | 3-methoxy-4-((3-(7-((tetrahydro-2H-pyran-4-yl)amino)-3-(thiazol-4-yl)benzo[b]thiophen-2-yl)prop-2-yn-1- |
| | yl)amino)benzenesulfonamide; |
| 87 | 3-methoxy-4-((3-(7-((tetrahydro-2H-pyran-4-yl)amino)-3-(2,2,2-trifluoroethyl)benzo[b]thiophen-2-yl)prop- |
| | 2-yn-1-yl)amino)benzenesulfonamide; |
| 88 | 4-((3-(3-(cyclopropylmethyl)-7-((tetrahydro-2H-pyran-4-yl)amino)benzo[b]thiophen-2-yl)prop-2-yn-1- |
| | yl)amino)-3-methoxybenzenesulfonamide; |
| 89 | 4-((3-(3-(2,2-difluoroethyl)-7-((tetrahydro-2H-pyran-4-yl)amino)benzo[b]thiophen-2-yl)prop-2-yn-1- |
| | yl)amino)-3-methoxybenzenesulfonamide; |
| 90 | 4-((3-(3-(1H-pyrrol-1-yl)-7-((tetrahydro-2H-pyran-4-yl)amino)benzo[b]thiophen-2-yl)prop-2-yn-1- |
| | yl)amino)-3-methoxybenzenesulfonamide; |
| 91 | 4-((3-(3-ethyl-7-((tetrahydro-2H-pyran-4-yl)amino)benzo[b]thiophen-2-yl)prop-2-yn-1-yl)amino)-3- |
| | methoxybenzenesulfonamide; |
| 92 | 2-(7-((3,3-difluoro-1-methylpiperidin-4-yl)amino)-2-(3-((5-fluoro-2-methoxy-4- |
| | (methylsulfonyl)phenyl)amino)prop-1-yn-1-yl)benzo[b]thiophen-3-yl)acetonitrile; |
| 93 | 3,3-difluoro-N-(2-(3-((5-fluoro-2-methoxy-4-(methylsulfonyl)phenyl)amino)prop-1-yn-1-yl)-3-(thiazol-4- |
| | yl)benzo[b]thiophen-7-yl)-1-methylpiperidin-4-amine; |
| 94 | 3,3-difluoro-N-(2-(3-((5-fluoro-2-methoxy-4-(methylsulfonyl)phenyl)amino)prop-1-yn-1-yl)-3-(2,2,2- |
| | trifluoroethyl)benzo[b]thiophen-7-yl)-1-methylpiperidin-4-amine; |
| 95 | N-(3-(cyclopropylmethyl)-2-(3-((5-fluoro-2-methoxy-4-(methylsulfonyl)phenyl)amino)prop-1-yn-1- |
| | yl)benzo[b]thiophen-7-yl)-3,3-difluoro-1-methylpiperidin-4-amine; |
| 96 | N-(3-(2,2-difluoroethyl)-2-(3-((5-fluoro-2-methoxy-4-(methylsulfonyl)phenyl)amino)prop-1-yn-1- |
| | yl)benzo[b]thiophen-7-yl)-3,3-difluoro-1-methylpiperidin-4-amine; |
| 97 | 3,3-difluoro-N-(2-(3-((5-fluoro-2-methoxy-4-(methylsulfonyl)phenyl)amino)prop-1-yn-1-yl)-3-(1H-pyrrol- |
| | 1-yl)benzo[b]thiophen-7-yl)-1-methylpiperidin-4-amine; |
| 98 | N-(3-ethyl-2-(3-((5-fluoro-2-methoxy-4-(methylsulfonyl)phenyl)amino)prop-1-yn-1-yl)benzo[b]thiophen- |
| | 7-yl)-3,3-difluoro-1-methylpiperidin-4-amine; |
| 99 | 2-(7-((1,1-dioxidotetrahydro-2H-thiopyran-4-yl)amino)-2-(3-((5-fluoro-2-methoxy-4- |
| | (methylsulfonyl)phenyl)amino)prop-1-yn-1-yl)benzo[b]thiophen-3-yl)acetonitrile; |
| 100 | 4-((2-(3-((5-fluoro-2-methoxy-4-(methylsulfonyl)phenyl)amino)prop-1-yn-1-yl)-3-(thiazol-4- |
| | yl)benzo[b]thiophen-7-yl)amino)tetrahydro-2H-thiopyran 1,1-dioxide; |
| 101 | 4-((2-(3-((5-fluoro-2-methoxy-4-(methylsulfonyl)phenyl)amino)prop-1-yn-1-yl)-3-(2,2,2- |
| | trifluoroethyl)benzo[b]thiophen-7-yl)amino)tetrahydro-2H-thiopyran 1,1-dioxide; |
| 102 | 4-((3-(cyclopropylmethyl)-2-(3-((5-fluoro-2-methoxy-4-(methylsulfonyl)phenyl)amino)prop-1-yn-1- |
| | yl)benzo[b]thiophen-7-yl)amino)tetrahydro-2H-thiopyran 1,1-dioxide; |
| 103 | 4-((3-(2,2-difluoroethyl)-2-(3-((5-fluoro-2-methoxy-4-(methylsulfonyl)phenyl)amino)prop-1-yn-1- |
| | yl)benzo[b]thiophen-7-yl)amino)tetrahydro-2H-thiopyran 1,1-dioxide; |
| 104 | 4-((2-(3-((5-fluoro-2-methoxy-4-(methylsulfonyl)phenyl)amino)prop-1-yn-1-yl)-3-(1H-pyrrol-1- |
| | yl)benzo[b]thiophen-7-yl)amino)tetrahydro-2H-thiopyran 1,1-dioxide; |
| 105 | 4-((3-ethyl-2-(3-((5-fluoro-2-methoxy-4-(methylsulfonyl)phenyl)amino)prop-1-yn-1-yl)benzo[b]thiophen- |
| | 7-yl)amino)tetrahydro-2H-thiopyran 1,1-dioxide; |
| 106 | 2-(7-(((3R,4S)-3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophen-3-yl)acetonitrile; |
| 107 | (3R,4S)-3-fluoro-N-(2-(3-((5-fluoro-2-methoxy-4-(methylsulfonyl)phenyl)amino)prop-1-yn-1-yl)-3- |
| | (thiazol-4-yl)benzo[b]thiophen-7-yl)-1-methylpiperidin-4-amine; |
| 108 | (3R,4S)-3-fluoro-N-(2-(3-((5-fluoro-2-methoxy-4-(methylsulfonyl)phenyl)amino)prop-1-yn-1-yl)-3-(2,2,2- |
| | trifluoroethyl)benzo[b]thiophen-7-yl)-1-methylpiperidin-4-amine; |
| 109 | (3R,4S)-N-(3-(cyclopropylmethyl)-2-(3-((5-fluoro-2-methoxy-4-(methylsulfonyl)phenyl)amino)prop-1-yn- |
| | 1-yl)benzo[b]thiophen-7-yl)-3-fluoro-1-methylpiperidin-4-amine; |
| 110 | (3R,4S)-N-(3-(2,2-difluoroethyl)-2-(3-((5-fluoro-2-methoxy-4-(methylsulfonyl)phenyl)amino)prop-1-yn-1- |
| | yl)benzo[b]thiophen-7-yl)-3-fluoro-1-methylpiperidin-4-amine; |
| 111 | (3R,4S)-3-fluoro-N-(2-(3-((5-fluoro-2-methoxy-4-(methylsulfonyl)phenyl)amino)prop-1-yn-1-yl)-3-(1H- |
| | pyrrol-1-yl)benzo[b]thiophen-7-yl)-1-methylpiperidin-4-amine; |
| 112 | (3R,4S)-N-(3-ethyl-2-(3-((5-fluoro-2-methoxy-4-(methylsulfonyl)phenyl)amino)prop-1-yn-1- |
| | yl)benzo[b]thiophen-7-yl)-3-fluoro-1-methylpiperidin-4-amine; |
| 113 | 2-(7-((3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophen-3-yl)acetonitrile; |
| 114 | 3-fluoro-N-(2-(3-((5-fluoro-2-methoxy-4-(methylsulfonyl)phenyl)amino)prop-1-yn-1-yl)-3-(thiazol-4- |
| | yl)benzo[b]thiophen-7-yl)-1-methylpiperidin-4-amine; |
| 115 | 3-fluoro-N-(2-(3-((5-fluoro-2-methoxy-4-(methylsulfonyl)phenyl)amino)prop-1-yn-1-yl)-3-(2,2,2- |
| | trifluoroethyl)benzo[b]thiophen-7-yl)-1-methylpiperidin-4-amine; |
| 116 | N-(3-(cyclopropylmethyl)-2-(3-((5-fluoro-2-methoxy-4-(methylsulfonyl)phenyl)amino)prop-1-yn-1- |
| | yl)benzo[b]thiophen-7-yl)-3-fluoro-1-methylpiperidin-4-amine; |
| 117 | N-(3-(2,2-difluoroethyl)-2-(3-((5-fluoro-2-methoxy-4-(methylsulfonyl)phenyl)amino)prop-1-yn-1- |
| | yl)benzo[b]thiophen-7-yl)-3-fluoro-1-methylpiperidin-4-amine; |
| 118 | 3-fluoro-N-(2-(3-((5-fluoro-2-methoxy-4-(methylsulfonyl)phenyl)amino)prop-1-yn-1-yl)-3-(1H-pyrrol-1- |
| | yl)benzo[b]thiophen-7-yl)-1-methylpiperidin-4-amine; |
| 119 | N-(3-ethyl-2-(3-((5-fluoro-2-methoxy-4-(methylsulfonyl)phenyl)amino)prop-1-yn-1-yl)benzo[b]thiophen- |
| | 7-yl)-3-fluoro-1-methylpiperidin-4-amine; |
| 120 | 2-(7-((1-(dimethylglycyl)piperidin-4-yl)amino)-2-(3-((5-fluoro-2-methoxy-4- |
| | (methylsulfonyl)phenyl)amino)prop-1-yn-1-yl)benzo[b]thiophen-3-yl)acetonitrile; |
| 121 | 2-(dimethylamino)-1-(4-((2-(3-((5-fluoro-2-methoxy-4-(methylsulfonyl)phenyl)amino)prop-1-yn-1-yl)-3- |
| | (thiazol-4-yl)benzo[b]thiophen-7-yl)amino)piperidin-1-yl)ethan-1-one; |
| 122 | 2-(dimethylamino)-1-(4-((2-(3-((5-fluoro-2-methoxy-4-(methylsulfonyl)phenyl)amino)prop-1-yn-1-yl)-3- |
| | (2,2,2-trifluoroethyl)benzo[b]thiophen-7-yl)amino)piperidin-1-yl)ethan-1-one; |
| 123 | 1-(4-((3-(cyclopropylmethyl)-2-(3-((5-fluoro-2-methoxy-4-(methylsulfonyl)phenyl)amino)prop-1-yn-1- |
| | yl)benzo[b]thiophen-7-yl)amino)piperidin-1-yl)-2-(dimethylamino)ethan-1-one; |
| 124 | 1-(4-((3-(2,2-difluoroethyl)-2-(3-((5-fluoro-2-methoxy-4-(methylsulfonyl)phenyl)amino)prop-1-yn-1- |
| | yl)benzo[b]thiophen-7-yl)amino)piperidin-1-yl)-2-(dimethylamino)ethan-1-one; |
| 125 | 2-(dimethylamino)-1-(4-((2-(3-((5-fluoro-2-methoxy-4-(methylsulfonyl)phenyl)amino)prop-1-yn-1-yl)-3- |
| | (1H-pyrrol-1-yl)benzo[b]thiophen-7-yl)amino)piperidin-1-yl)ethan-1-one; |
| 126 | 2-(dimethylamino)-1-(4-((3-ethyl-2-(3-((5-fluoro-2-methoxy-4-(methylsulfonyl)phenyl)amino)prop-1-yn-1- |
| | yl)benzo[b]thiophen-7-yl)amino)piperidin-1-yl)ethan-1-one; |
| 127 | 2-(7-((3-fluoro-1-(2-hydroxy-3-methoxypropyl)piperidin-4-yl)amino)-2-(3-((5-fluoro-2-methoxy-4- |
| | (methylsulfonyl)phenyl)amino)prop-1-yn-1-yl)benzo[b]thiophen-3-yl)acetonitrile; |
| 128 | 1-(3-fluoro-4-((2-(3-((5-fluoro-2-methoxy-4-(methylsulfonyl)phenyl)amino)prop-1-yn-1-yl)-3-(thiazol-4- |
| | yl)benzo[b]thiophen-7-yl)amino)piperidin-1-yl)-3-methoxypropan-2-ol; |
| 129 | 1-(3-fluoro-4-((2-(3-((5-fluoro-2-methoxy-4-(methylsulfonyl)phenyl)amino)prop-1-yn-1-yl)-3-(2,2,2- |
| | trifluoroethyl)benzo[b]thiophen-7-yl)amino)piperidin-1-yl)-3-methoxypropan-2-ol; |
| 130 | 1-(4-((3-(cyclopropylmethyl)-2-(3-((5-fluoro-2-methoxy-4-(methylsulfonyl)phenyl)amino)prop-1-yn-1- |
| | yl)benzo[b]thiophen-7-yl)amino)-3-fluoropiperidin-1-yl)-3-methoxypropan-2-ol; |
| 131 | 1-(4-((3-(2,2-difluoroethyl)-2-(3-((5-fluoro-2-methoxy-4-(methylsulfonyl)phenyl)amino)prop-1-yn-1- |
| | yl)benzo[b]thiophen-7-yl)amino)-3-fluoropiperidin-1-yl)-3-methoxypropan-2-ol; |
| 132 | 1-(3-fluoro-4-((2-(3-((5-fluoro-2-methoxy-4-(methylsulfonyl)phenyl)amino)prop-1-yn-1-yl)-3-(1H-pyrrol- |
| | 1-yl)benzo[b]thiophen-7-yl)amino)piperidin-1-yl)-3-methoxypropan-2-ol; |
| 133 | 1-(4-((3-ethyl-2-(3-((5-fluoro-2-methoxy-4-(methylsulfonyl)phenyl)amino)prop-1-yn-1- |
| | yl)benzo[b]thiophen-7-yl)amino)-3-fluoropiperidin-1-yl)-3-methoxypropan-2-ol; |
| 134 | 2-(7-(((3R,4R)-3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophen-3-yl)acetonitrile; |
| 135 | (3R,4R)-3-fluoro-N-(2-(3-((5-fluoro-2-methoxy-4-(methylsulfonyl)phenyl)amino)prop-1-yn-1-yl)-3- |
| | (thiazol-4-yl)benzo[b]thiophen-7-yl)-1-methylpiperidin-4-amine; |
| 136 | (3R,4R)-3-fluoro-N-(2-(3-((5-fluoro-2-methoxy-4-(methylsulfonyl)phenyl)amino)prop-1-yn-1-yl)-3-(2,2,2- |
| | trifluoroethyl)benzo[b]thiophen-7-yl)-1-methylpiperidin-4-amine; |
| 137 | (3R,4R)-N-(3-(cyclopropylmethyl)-2-(3-((5-fluoro-2-methoxy-4-(methylsulfonyl)phenyl)amino)prop-1-yn- |
| | 1-yl)benzo[b]thiophen-7-yl)-3-fluoro-1-methylpiperidin-4-amine; |
| 138 | (3R,4R)-N-(3-(2,2-difluoroethyl)-2-(3-((5-fluoro-2-methoxy-4-(methylsulfonyl)phenyl)amino)prop-1-yn-1- |
| | yl)benzo[b]thiophen-7-yl)-3-fluoro-1-methylpiperidin-4-amine; |
| 139 | (3R,4R)-3-fluoro-N-(2-(3-((5-fluoro-2-methoxy-4-(methylsulfonyl)phenyl)amino)prop-1-yn-1-yl)-3-(1H- |
| | pyrrol-1-yl)benzo[b]thiophen-7-yl)-1-methylpiperidin-4-amine; |
| 140 | (3R,4R)-N-(3-ethyl-2-(3-((5-fluoro-2-methoxy-4-(methylsulfonyl)phenyl)amino)prop-1-yn-1- |
| | yl)benzo[b]thiophen-7-yl)-3-fluoro-1-methylpiperidin-4-amine; |
| 141 | 2-(7-(((3S,4S)-3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophen-3-yl)acetonitrile; |
| 142 | (3S,4S)-3-fluoro-N-(2-(3-((5-fluoro-2-methoxy-4-(methylsulfonyl)phenyl)amino)prop-1-yn-1-yl)-3- |
| | (thiazol-4-yl)benzo[b]thiophen-7-yl)-1-methylpiperidin-4-amine; |
| 143 | (3S,4S)-3-fluoro-N-(2-(3-((5-fluoro-2-methoxy-4-(methylsulfonyl)phenyl)amino)prop-1-yn-1-yl)-3-(2,2,2- |
| | trifluoroethyl)benzo[b]thiophen-7-yl)-1-methylpiperidin-4-amine; |
| 144 | (3S,4S)-N-(3-(cyclopropylmethyl)-2-(3-((5-fluoro-2-methoxy-4-(methylsulfonyl)phenyl)amino)prop-1-yn- |
| | 1-yl)benzo[b]thiophen-7-yl)-3-fluoro-1-methylpiperidin-4-amine; |
| 145 | (3S,4S)-N-(3-(2,2-difluoroethyl)-2-(3-((5-fluoro-2-methoxy-4-(methylsulfonyl)phenyl)amino)prop-1-yn-1- |
| | yl)benzo[b]thiophen-7-yl)-3-fluoro-1-methylpiperidin-4-amine; |
| 146 | (3S,4S)-3-fluoro-N-(2-(3-((5-fluoro-2-methoxy-4-(methylsulfonyl)phenyl)amino)prop-1-yn-1-yl)-3-(1H- |
| | pyrrol-1-yl)benzo[b]thiophen-7-yl)-1-methylpiperidin-4-amine; |
| 147 | (3S,4S)-N-(3-ethyl-2-(3-((5-fluoro-2-methoxy-4-(methylsulfonyl)phenyl)amino)prop-1-yn-1- |
| | yl)benzo[b]thiophen-7-yl)-3-fluoro-1-methylpiperidin-4-amine; |
| 148 | 2-(7-((4-(dimethylamino)cyclohexyl)amino)-2-(3-((5-fluoro-2-methoxy-4- |
| | (methylsulfonyl)phenyl)amino)prop-1-yn-1-yl)benzo[b]thiophen-3-yl)acetonitrile; |
| 149 | N1-(2-(3-((5-fluoro-2-methoxy-4-(methylsulfonyl)phenyl)amino)prop-1-yn-1-yl)-3-(thiazol-4- |
| | yl)benzo[b]thiophen-7-yl)-N4,N4-dimethylcyclohexane-1,4-diamine; |
| 150 | N1-(2-(3-((5-fluoro-2-methoxy-4-(methylsulfonyl)phenyl)amino)prop-1-yn-1-yl)-3-(2,2,2- |
| | trifluoroethyl)benzo[b]thiophen-7-yl)-N4,N4-dimethylcyclohexane-1,4-diamine; |
| 151 | N1-(3-(cyclopropylmethyl)-2-(3-((5-fluoro-2-methoxy-4-(methylsulfonyl)phenyl)amino)prop-1-yn-1- |
| | yl)benzo[b]thiophen-7-yl)-N4,N4-dimethylcyclohexane-1,4-diamine; |
| 152 | N1-(3-(2,2-difluoroethyl)-2-(3-((5-fluoro-2-methoxy-4-(methylsulfonyl)phenyl)amino)prop-1-yn-1- |
| | yl)benzo[b]thiophen-7-yl)-N4,N4-dimethylcyclohexane-1,4-diamine; |
| 153 | N1-(2-(3-((5-fluoro-2-methoxy-4-(methylsulfonyl)phenyl)amino)prop-1-yn-1-yl)-3-(1H-pyrrol-1- |
| | yl)benzo[b]thiophen-7-yl)-N4,N4-dimethylcyclohexane-1,4-diamine; |
| 154 | N1-(3-ethyl-2-(3-((5-fluoro-2-methoxy-4-(methylsulfonyl)phenyl)amino)prop-1-yn-1-yl)benzo[b]thiophen- |
| | 7-yl)-N4,N4-dimethylcyclohexane-1,4-diamine; |
| 155 | 2-(7-((2-(dimethylamino)ethyl)amino)-2-(3-((5-fluoro-2-methoxy-4-(methylsulfonyl)phenyl)amino)prop-1- |
| | yn-1-yl)benzo[b]thiophen-3-yl)acetonitrile; |
| 156 | N1-(2-(3-((5-fluoro-2-methoxy-4-(methylsulfonyl)phenyl)amino)prop-1-yn-1-yl)-3-(thiazol-4- |
| | yl)benzo[b]thiophen-7-yl)-N2,N2-dimethylethane-1,2-diamine; |
| 157 | N1-(2-(3-((5-fluoro-2-methoxy-4-(methylsulfonyl)phenyl)amino)prop-1-yn-1-yl)-3-(2,2,2- |
| | trifluoroethyl)benzo[b]thiophen-7-yl)-N2,N2-dimethylethane-1,2-diamine; |
| 158 | N1-(3-(cyclopropylmethyl)-2-(3-((5-fluoro-2-methoxy-4-(methylsulfonyl)phenyl)amino)prop-1-yn-1- |
| | yl)benzo[b]thiophen-7-yl)-N2,N2-dimethylethane-1,2-diamine; |
| 159 | N1-(3-(2,2-difluoroethyl)-2-(3-((5-fluoro-2-methoxy-4-(methylsulfonyl)phenyl)amino)prop-1-yn-1- |
| | yl)benzo[b]thiophen-7-yl)-N2,N2-dimethylethane-1,2-diamine; |
| 160 | N1-(2-(3-((5-fluoro-2-methoxy-4-(methylsulfonyl)phenyl)amino)prop-1-yn-1-yl)-3-(1H-pyrrol-1- |
| | yl)benzo[b]thiophen-7-yl)-N2,N2-dimethylethane-1,2-diamine; |
| 161 | N1-(3-ethyl-2-(3-((5-fluoro-2-methoxy-4-(methylsulfonyl)phenyl)amino)prop-1-yn-1-yl)benzo[b]thiophen- |
| | 7-yl)-N2,N2-dimethylethane-1,2-diamine; |
| 162 | 2-(7-(((3S,4R)-3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophen-3-yl)acetonitrile; |
| 163 | (3S,4R)-3-fluoro-N-(2-(3-((5-fluoro-2-methoxy-4-(methylsulfonyl)phenyl)amino)prop-1-yn-1-yl)-3- |
| | (thiazol-4-yl)benzo[b]thiophen-7-yl)-1-methylpiperidin-4-amine; |
| 164 | (3S,4R)-3-fluoro-N-(2-(3-((5-fluoro-2-methoxy-4-(methylsulfonyl)phenyl)amino)prop-1-yn-1-yl)-3-(2,2,2- |
| | trifluoroethyl)benzo[b]thiophen-7-yl)-1-methylpiperidin-4-amine; |
| 165 | (3S,4R)-N-(3-(cyclopropylmethyl)-2-(3-((5-fluoro-2-methoxy-4-(methylsulfonyl)phenyl)amino)prop-1-yn- |
| | 1-yl)benzo[b]thiophen-7-yl)-3-fluoro-1-methylpiperidin-4-amine; |
| 166 | (3S,4R)-N-(3-(2,2-difluoroethyl)-2-(3-((5-fluoro-2-methoxy-4-(methylsulfonyl)phenyl)amino)prop-1-yn-1- |
| | yl)benzo[b]thiophen-7-yl)-3-fluoro-1-methylpiperidin-4-amine; |
| 167 | (3S,4R)-3-fluoro-N-(2-(3-((5-fluoro-2-methoxy-4-(methylsulfonyl)phenyl)amino)prop-1-yn-1-yl)-3-(1H- |
| | pyrrol-1-yl)benzo[b]thiophen-7-yl)-1-methylpiperidin-4-amine; |
| 168 | (3S,4R)-N-(3-ethyl-2-(3-((5-fluoro-2-methoxy-4-(methylsulfonyl)phenyl)amino)prop-1-yn-1- |
| | yl)benzo[b]thiophen-7-yl)-3-fluoro-1-methylpiperidin-4-amine; |
| 169 | 2-(2-(3-((5-fluoro-2-methoxy-4-(methylsulfonyl)phenyl)amino)prop-1-yn-1-yl)-7-((1-methylpiperidin-4- |
| | yl)amino)benzo[b]thiophen-3-yl)acetonitrile; |
| 170 | N-(2-(3-((5-fluoro-2-methoxy-4-(methylsulfonyl)phenyl)amino)prop-1-yn-1-yl)-3-(thiazol-4- |
| | yl)benzo[b]thiophen-7-yl)-1-methylpiperidin-4-amine; |
| 171 | N-(2-(3-((5-fluoro-2-methoxy-4-(methylsulfonyl)phenyl)amino)prop-1-yn-1-yl)-3-(2,2,2- |
| | trifluoroethyl)benzo[b]thiophen-7-yl)-1-methylpiperidin-4-amine; |
| 172 | N-(3-(cyclopropylmethyl)-2-(3-((5-fluoro-2-methoxy-4-(methylsulfonyl)phenyl)amino)prop-1-yn-1- |
| | yl)benzo[b]thiophen-7-yl)-1-methylpiperidin-4-amine; |
| 173 | N-(3-(2,2-difluoroethyl)-2-(3-((5-fluoro-2-methoxy-4-(methylsulfonyl)phenyl)amino)prop-1-yn-1- |
| | yl)benzo[b]thiophen-7-yl)-1-methylpiperidin-4-amine; |
| 174 | N-(2-(3-((5-fluoro-2-methoxy-4-(methylsulfonyl)phenyl)amino)prop-1-yn-1-yl)-3-(1H-pyrrol-1- |
| | yl)benzo[b]thiophen-7-yl)-1-methylpiperidin-4-amine; |
| 175 | N-(3-ethyl-2-(3-((5-fluoro-2-methoxy-4-(methylsulfonyl)phenyl)amino)prop-1-yn-1-yl)benzo[b]thiophen- |
| | 7-yl)-1-methylpiperidin-4-amine; |
| 176 | 2-(2-(3-((5-fluoro-2-methoxy-4-(methylsulfonyl)phenyl)amino)prop-1-yn-1-yl)-7-((tetrahydro-2H-pyran-4- |
| | yl)amino)benzo[b]thiophen-3-yl)acetonitrile; |
| 177 | N-(2-(3-((5-fluoro-2-methoxy-4-(methylsulfonyl)phenyl)amino)prop-1-yn-1-yl)-3-(thiazol-4- |
| | yl)benzo[b]thiophen-7-yl)tetrahydro-2H-pyran-4-amine; |
| 178 | N-(2-(3-((5-fluoro-2-methoxy-4-(methylsulfonyl)phenyl)amino)prop-1-yn-1-yl)-3-(2,2,2- |
| | trifluoroethyl)benzo[b]thiophen-7-yl)tetrahydro-2H-pyran-4-amine; |
| 179 | N-(3-(cyclopropylmethyl)-2-(3-((5-fluoro-2-methoxy-4-(methylsulfonyl)phenyl)amino)prop-1-yn-1- |
| | yl)benzo[b]thiophen-7-yl)tetrahydro-2H-pyran-4-amine; |
| 180 | N-(3-(2,2-difluoroethyl)-2-(3-((5-fluoro-2-methoxy-4-(methylsulfonyl)phenyl)amino)prop-1-yn-1- |
| | yl)benzo[b]thiophen-7-yl)tetrahydro-2H-pyran-4-amine; |
| 181 | N-(2-(3-((5-fluoro-2-methoxy-4-(methylsulfonyl)phenyl)amino)prop-1-yn-1-yl)-3-(1H-pyrrol-1- |
| | yl)benzo[b]thiophen-7-yl)tetrahydro-2H-pyran-4-amine; |
| 182 | N-(3-ethyl-2-(3-((5-fluoro-2-methoxy-4-(methylsulfonyl)phenyl)amino)prop-1-yn-1-yl)benzo[b]thiophen- |
| | 7-yl)tetrahydro-2H-pyran-4-amine; |
| 183 | 4-((3-(3-(cyanomethyl)-7-((3,3-difluoro-1-methylpiperidin-4-yl)amino)benzo[b]thiophen-2-yl)prop-2-yn-1- |
| | yl)amino)-3-methoxy-N-methylbenzamide; |
| 184 | 4-((3-(7-((3,3-difluoro-1-methylpiperidin-4-yl)amino)-3-(thiazol-4-yl)benzo[b]thiophen-2-yl)prop-2-yn-1- |
| | yl)amino)-3-methoxy-N-methylbenzamide; |
| 185 | 4-((3-(7-((3,3-difluoro-1-methylpiperidin-4-yl)amino)-3-(2,2,2-trifluoroethyl)benzo[b]thiophen-2-yl)prop- |
| | 2-yn-1-yl)amino)-3-methoxy-N-methylbenzamide; |
| 186 | 4-((3-(3-(cyclopropylmethyl)-7-((3,3-difluoro-1-methylpiperidin-4-yl)amino)benzo[b]thiophen-2-yl)prop-2- |
| | yn-1-yl)amino)-3-methoxy-N-methylbenzamide; |
| 187 | 4-((3-(7-((3,3-difluoro-1-methylpiperidin-4-yl)amino)-3-(2,2-difluoroethyl)benzo[b]thiophen-2-yl)prop-2- |
| | yn-1-yl)amino)-3-methoxy-N-methylbenzamide; |
| 188 | 4-((3-(7-((3,3-difluoro-1-methylpiperidin-4-yl)amino)-3-(1H-pyrrol-1-yl)benzo[b]thiophen-2-yl)prop-2-yn- |
| | 1-yl)amino)-3-methoxy-N-methylbenzamide; |
| 189 | 4-((3-(7-((3,3-difluoro-1-methylpiperidin-4-yl)amino)-3-ethylbenzo[b]thiophen-2-yl)prop-2-yn-1- |
| | yl)amino)-3-methoxy-N-methylbenzamide; |
| 190 | 4-((3-(3-(cyanomethyl)-7-((1,1-dioxidotetrahydro-2H-thiopyran-4-yl)amino)benzo[b]thiophen-2-yl)prop-2- |
| | yn-1-yl)amino)-3-methoxy-N-methylbenzamide; |
| 191 | 4-((3-(7-((1,1-dioxidotetrahydro-2H-thiopyran-4-yl)amino)-3-(thiazol-4-yl)benzo[b]thiophen-2-yl)prop-2- |
| | yn-1-yl)amino)-3-methoxy-N-methylbenzamide; |
| 192 | 4-((3-(7-((1,1-dioxidotetrahydro-2H-thiopyran-4-yl)amino)-3-(2,2,2-trifluoroethyl)benzo[b]thiophen-2- |
| | yl)prop-2-yn-1-yl)amino)-3-methoxy-N-methylbenzamide; |
| 193 | 4-((3-(3-(cyclopropylmethyl)-7-((1,1-dioxidotetrahydro-2H-thiopyran-4-yl)amino)benzo[b]thiophen-2- |
| | yl)prop-2-yn-1-yl)amino)-3-methoxy-N-methylbenzamide; |
| 194 | 4-((3-(3-(2,2-difluoroethyl)-7-((1,1-dioxidotetrahydro-2H-thiopyran-4-yl)amino)benzo[b]thiophen-2- |
| | yl)prop-2-yn-1-yl)amino)-3-methoxy-N-methylbenzamide; |
| 195 | 4-((3-(7-((1,1-dioxidotetrahydro-2H-thiopyran-4-yl)amino)-3-(1H-pyrrol-1-yl)benzo[b]thiophen-2-yl)prop- |
| | 2-yn-1-yl)amino)-3-methoxy-N-methylbenzamide; |
| 196 | 4-((3-(7-((1,1-dioxidotetrahydro-2H-thiopyran-4-yl)amino)-3-ethylbenzo[b]thiophen-2-yl)prop-2-yn-1- |
| | yl)amino)-3-methoxy-N-methylbenzamide; |
| 197 | 4-((3-(3-(cyanomethyl)-7-(((3R,4S)-3-fluoro-1-methylpiperidin-4-yl)amino)benzo[b]thiophen-2-yl)prop-2- |
| | yn-1-yl)amino)-3-methoxy-N-methylbenzamide; |
| 198 | 4-((3-(7-(((3R,4S)-3-fluoro-1-methylpiperidin-4-yl)amino)-3-(thiazol-4-yl)benzo[b]thiophen-2-yl)prop-2- |
| | yn-1-yl)amino)-3-methoxy-N-methylbenzamide; |
| 199 | 4-((3-(7-(((3R,4S)-3-fluoro-1-methylpiperidin-4-yl)amino)-3-(2,2,2-trifluoroethyl)benzo[b]thiophen-2- |
| | yl)prop-2-yn-1-yl)amino)-3-methoxy-N-methylbenzamide; |
| 200 | 4-((3-(3-(cyclopropylmethyl)-7-(((3R,4S)-3-fluoro-1-methylpiperidin-4-yl)amino)benzo[b]thiophen-2- |
| | yl)prop-2-yn-1-yl)amino)-3-methoxy-N-methylbenzamide; |
| 201 | 4-((3-(3-(2,2-difluoroethyl)-7-(((3R,4S)-3-fluoro-1-methylpiperidin-4-yl)amino)benzo[b]thiophen-2- |
| | yl)prop-2-yn-1-yl)amino)-3-methoxy-N-methylbenzamide; |
| 202 | 4-((3-(7-(((3R,4S)-3-fluoro-1-methylpiperidin-4-yl)amino)-3-(1H-pyrrol-1-yl)benzo[b]thiophen-2-yl)prop- |
| | 2-yn-1-yl)amino)-3-methoxy-N-methylbenzamide; |
| 203 | 4-((3-(3-ethyl-7-(((3R,4S)-3-fluoro-1-methylpiperidin-4-yl)amino)benzo[b]thiophen-2-yl)prop-2-yn-1- |
| | yl)amino)-3-methoxy-N-methylbenzamide; |
| 204 | 4-((3-(3-(cyanomethyl)-7-((3-fluoro-1-methylpiperidin-4-yl)amino)benzo[b]thiophen-2-yl)prop-2-yn-1- |
| | yl)amino)-3-methoxy-N-methylbenzamide; |
| 205 | 4-((3-(7-((3-fluoro-1-methylpiperidin-4-yl)amino)-3-(thiazol-4-yl)benzo[b]thiophen-2-yl)prop-2-yn-1- |
| | yl)amino)-3-methoxy-N-methylbenzamide; |
| 206 | 4-((3-(7-((3-fluoro-1-methylpiperidin-4-yl)amino)-3-(2,2,2-trifluoroethyl)benzo[b]thiophen-2-yl)prop-2-yn- |
| | 1-yl)amino)-3-methoxy-N-methylbenzamide; |
| 207 | 4-((3-(3-(cyclopropylmethyl)-7-((3-fluoro-1-methylpiperidin-4-yl)amino)benzo[b]thiophen-2-yl)prop-2-yn- |
| | 1-yl)amino)-3-methoxy-N-methylbenzamide; |
| 208 | 4-((3-(3-(2,2-difluoroethyl)-7-((3-fluoro-1-methylpiperidin-4-yl)amino)benzo[b]thiophen-2-yl)prop-2-yn-1- |
| | yl)amino)-3-methoxy-N-methylbenzamide; |
| 209 | 4-((3-(7-((3-fluoro-1-methylpiperidin-4-yl)amino)-3-(1H-pyrrol-1-yl)benzo[b]thiophen-2-yl)prop-2-yn-1- |
| | yl)amino)-3-methoxy-N-methylbenzamide; |
| 210 | 4-((3-(3-ethyl-7-((3-fluoro-1-methylpiperidin-4-yl)amino)benzo[b]thiophen-2-yl)prop-2-yn-1-yl)amino)-3- |
| | methoxy-N-methylbenzamide; |
| 211 | 4-((3-(3-(cyanomethyl)-7-((1-(dimethylglycyl)piperidin-4-yl)amino)benzo[b]thiophen-2-yl)prop-2-yn-1- |
| | yl)amino)-3-methoxy-N-methylbenzamide; |
| 212 | 4-((3-(7-((1-(dimethylglycyl)piperidin-4-yl)amino)-3-(thiazol-4-yl)benzo[b]thiophen-2-yl)prop-2-yn-1- |
| | yl)amino)-3-methoxy-N-methylbenzamide; |
| 213 | 4-((3-(7-((1-(dimethylglycyl)piperidin-4-yl)amino)-3-(2,2,2-trifluoroethyl)benzo[b]thiophen-2-yl)prop-2- |
| | yn-1-yl)amino)-3-methoxy-N-methylbenzamide; |
| 214 | 4-((3-(3-(cyclopropylmethyl)-7-((1-(dimethylglycyl)piperidin-4-yl)amino)benzo[b]thiophen-2-yl)prop-2- |
| | yn-1-yl)amino)-3-methoxy-N-methylbenzamide; |
| 215 | 4-((3-(3-(2,2-difluoroethyl)-7-((1-(dimethylglycyl)piperidin-4-yl)amino)benzo[b]thiophen-2-yl)prop-2-yn- |
| | 1-yl)amino)-3-methoxy-N-methylbenzamide; |
| 216 | 4-((3-(7-((1-(dimethylglycyl)piperidin-4-yl)amino)-3-(1H-pyrrol-1-yl)benzo[b]thiophen-2-yl)prop-2-yn-1- |
| | yl)amino)-3-methoxy-N-methylbenzamide; |
| 217 | 4-((3-(7-((1-(dimethylglycyl)piperidin-4-yl)amino)-3-ethylbenzo[b]thiophen-2-yl)prop-2-yn-1-yl)amino)-3- |
| | methoxy-N-methylbenzamide; |
| 218 | 4-((3-(3-(cyanomethyl)-7-((3-fluoro-1-(2-hydroxy-3-methoxypropyl)piperidin-4- |
| | yl)amino)benzo[b]thiophen-2-yl)prop-2-yn-1-yl)amino)-3-methoxy-N-methylbenzamide; |
| 219 | 4-((3-(7-((3-fluoro-1-(2-hydroxy-3-methoxypropyl)piperidin-4-yl)amino)-3-(thiazol-4-yl)benzo[b]thiophen- |
| | 2-yl)prop-2-yn-1-yl)amino)-3-methoxy-N-methylbenzamide; |
| 220 | 4-((3-(7-((3-fluoro-1-(2-hydroxy-3-methoxypropyl)piperidin-4-yl)amino)-3-(2,2,2- |
| | trifluoroethyl)benzo[b]thiophen-2-yl)prop-2-yn-1-yl)amino)-3-methoxy-N-methylbenzamide; |
| 221 | 4-((3-(3-(cyclopropylmethyl)-7-((3-fluoro-1-(2-hydroxy-3-methoxypropyl)piperidin-4- |
| | yl)amino)benzo[b]thiophen-2-yl)prop-2-yn-1-yl)amino)-3-methoxy-N-methylbenzamide; |
| 222 | 4-((3-(3-(2,2-difluoroethyl)-7-((3-fluoro-1-(2-hydroxy-3-methoxypropyl)piperidin-4- |
| | yl)amino)benzo[b]thiophen-2-yl)prop-2-yn-1-yl)amino)-3-methoxy-N-methylbenzamide; |
| 223 | 4-((3-(7-((3-fluoro-1-(2-hydroxy-3-methoxypropyl)piperidin-4-yl)amino)-3-(1H-pyrrol-1- |
| | yl)benzo[b]thiophen-2-yl)prop-2-yn-1-yl)amino)-3-methoxy-N-methylbenzamide; |
| 224 | 4-((3-(3-ethyl-7-((3-fluoro-1-(2-hydroxy-3-methoxypropyl)piperidin-4-yl)amino)benzo[b]thiophen-2- |
| | yl)prop-2-yn-1-yl)amino)-3-methoxy-N-methylbenzamide; |
| 225 | 4-((3-(3-(cyanomethyl)-7-(((3R,4R)-3-fluoro-1-methylpiperidin-4-yl)amino)benzo[b]thiophen-2-yl)prop-2- |
| | yn-1-yl)amino)-3-methoxy-N-methylbenzamide; |
| 226 | 4-((3-(7-(((3R,4R)-3-fluoro-1-methylpiperidin-4-yl)amino)-3-(thiazol-4-yl)benzo[b]thiophen-2-yl)prop-2- |
| | yn-1-yl)amino)-3-methoxy-N-methylbenzamide; |
| 227 | 4-((3-(7-(((3R,4R)-3-fluoro-1-methylpiperidin-4-yl)amino)-3-(2,2,2-trifluoroethyl)benzo[b]thiophen-2- |
| | yl)prop-2-yn-1-yl)amino)-3-methoxy-N-methylbenzamide; |
| 228 | 4-((3-(3-(cyclopropylmethyl)-7-(((3R,4R)-3-fluoro-1-methylpiperidin-4-yl)amino)benzo[b]thiophen-2- |
| | yl)prop-2-yn-1-yl)amino)-3-methoxy-N-methylbenzamide; |
| 229 | 4-((3-(3-(2,2-difluoroethyl)-7-(((3R,4R)-3-fluoro-1-methylpiperidin-4-yl)amino)benzo[b]thiophen-2- |
| | yl)prop-2-yn-1-yl)amino)-3-methoxy-N-methylbenzamide; |
| 230 | 4-((3-(7-(((3R,4R)-3-fluoro-1-methylpiperidin-4-yl)amino)-3-(1H-pyrrol-1-yl)benzo[b]thiophen-2-yl)prop- |
| | 2-yn-1-yl)amino)-3-methoxy-N-methylbenzamide; |
| 231 | 4-((3-(3-ethyl-7-(((3R,4R)-3-fluoro-1-methylpiperidin-4-yl)amino)benzo[b]thiophen-2-yl)prop-2-yn-1- |
| | yl)amino)-3-methoxy-N-methylbenzamide; |
| 232 | 4-((3-(3-(cyanomethyl)-7-(((3S,4S)-3-fluoro-1-methylpiperidin-4-yl)amino)benzo[b]thiophen-2-yl)prop-2- |
| | yn-1-yl)amino)-3-methoxy-N-methylbenzamide; |
| 233 | 4-((3-(7-(((3S,4S)-3-fluoro-1-methylpiperidin-4-yl)amino)-3-(thiazol-4-yl)benzo[b]thiophen-2-yl)prop-2- |
| | yn-1-yl)amino)-3-methoxy-N-methylbenzamide; |
| 234 | 4-((3-(7-(((3S,4S)-3-fluoro-1-methylpiperidin-4-yl)amino)-3-(2,2,2-trifluoroethyl)benzo[b]thiophen-2- |
| | yl)prop-2-yn-1-yl)amino)-3-methoxy-N-methylbenzamide; |
| 235 | 4-((3-(3-(cyclopropylmethyl)-7-(((3S,4S)-3-fluoro-1-methylpiperidin-4-yl)amino)benzo[b]thiophen-2- |
| | yl)prop-2-yn-1-yl)amino)-3-methoxy-N-methylbenzamide; |
| 236 | 4-((3-(3-(2,2-difluoroethyl)-7-(((3S,4S)-3-fluoro-1-methylpiperidin-4-yl)amino)benzo[b]thiophen-2- |
| | yl)prop-2-yn-1-yl)amino)-3-methoxy-N-methylbenzamide; |
| 237 | 4-((3-(7-(((3S,4S)-3-fluoro-1-methylpiperidin-4-yl)amino)-3-(1H-pyrrol-1-yl)benzo[b]thiophen-2-yl)prop- |
| | 2-yn-1-yl)amino)-3-methoxy-N-methylbenzamide; |
| 238 | 4-((3-(3-ethyl-7-(((3S,4S)-3-fluoro-1-methylpiperidin-4-yl)amino)benzo[b]thiophen-2-yl)prop-2-yn-1- |
| | yl)amino)-3-methoxy-N-methylbenzamide; |
| 239 | 4-((3-(3-(cyanomethyl)-7-((4-(dimethylamino)cyclohexyl)amino)benzo[b]thiophen-2-yl)prop-2-yn-1- |
| | yl)amino)-3-methoxy-N-methylbenzamide; |
| 240 | 4-((3-(7-((4-(dimethylamino)cyclohexyl)amino)-3-(thiazol-4-yl)benzo[b]thiophen-2-yl)prop-2-yn-1- |
| | yl)amino)-3-methoxy-N-methylbenzamide; |
| 241 | 4-((3-(7-((4-(dimethylamino)cyclohexyl)amino)-3-(2,2,2-trifluoroethyl)benzo[b]thiophen-2-yl)prop-2-yn-1- |
| | yl)amino)-3-methoxy-N-methylbenzamide; |
| 242 | 4-((3-(3-(cyclopropylmethyl)-7-((4-(dimethylamino)cyclohexyl)amino)benzo[b]thiophen-2-yl)prop-2-yn-1- |
| | yl)amino)-3-methoxy-N-methylbenzamide; |
| 243 | 4-((3-(3-(2,2-difluoroethyl)-7-((4-(dimethylamino)cyclohexyl)amino)benzo[b]thiophen-2-yl)prop-2-yn-1- |
| | yl)amino)-3-methoxy-N-methylbenzamide; |
| 244 | 4-((3-(7-((4-(dimethylamino)cyclohexyl)amino)-3-(1H-pyrrol-1-yl)benzo[b]thiophen-2-yl)prop-2-yn-1- |
| | yl)amino)-3-methoxy-N-methylbenzamide; |
| 245 | 4-((3-(7-((4-(dimethylamino)cyclohexyl)amino)-3-ethylbenzo[b]thiophen-2-yl)prop-2-yn-1-yl)amino)-3- |
| | methoxy-N-methylbenzamide; |
| 246 | 4-((3-(3-(cyanomethyl)-7-((2-(dimethylamino)ethyl)amino)benzo[b]thiophen-2-yl)prop-2-yn-1-yl)amino)- |
| | 3-methoxy-N-methylbenzamide; |
| 247 | 4-((3-(7-((2-(dimethylamino)ethyl)amino)-3-(thiazol-4-yl)benzo[b]thiophen-2-yl)prop-2-yn-1-yl)amino)-3- |
| | methoxy-N-methylbenzamide; |
| 248 | 4-((3-(7-((2-(dimethylamino)ethyl)amino)-3-(2,2,2-trifluoroethyl)benzo[b]thiophen-2-yl)prop-2-yn-1- |
| | yl)amino)-3-methoxy-N-methylbenzamide; |
| 249 | 4-((3-(3-(cyclopropylmethyl)-7-((2-(dimethylamino)ethyl)amino)benzo[b]thiophen-2-yl)prop-2-yn-1- |
| | yl)amino)-3-methoxy-N-methylbenzamide; |
| 250 | 4-((3-(3-(2,2-difluoroethyl)-7-((2-(dimethylamino)ethyl)amino)benzo[b]thiophen-2-yl)prop-2-yn-1- |
| | yl)amino)-3-methoxy-N-methylbenzamide; |
| 251 | 4-((3-(7-((2-(dimethylamino)ethyl)amino)-3-(1H-pyrrol-1-yl)benzo[b]thiophen-2-yl)prop-2-yn-1- |
| | yl)amino)-3-methoxy-N-methylbenzamide; |
| 252 | 4-((3-(7-((2-(dimethylamino)ethyl)amino)-3-ethylbenzo[b]thiophen-2-yl)prop-2-yn-1-yl)amino)-3- |
| | methoxy-N-methylbenzamide; |
| 253 | 4-((3-(3-(cyanomethyl)-7-(((3S,4R)-3-fluoro-1-methylpiperidin-4-yl)amino)benzo[b]thiophen-2-yl)prop-2- |
| | yn-1-yl)amino)-3-methoxy-N-methylbenzamide; |
| 254 | 4-((3-(7-(((3S,4R)-3-fluoro-1-methylpiperidin-4-yl)amino)-3-(thiazol-4-yl)benzo[b]thiophen-2-yl)prop-2- |
| | yn-1-yl)amino)-3-methoxy-N-methylbenzamide; |
| 255 | 4-((3-(7-(((3S,4R)-3-fluoro-1-methylpiperidin-4-yl)amino)-3-(2,2,2-trifluoroethyl)benzo[b]thiophen-2- |
| | yl)prop-2-yn-1-yl)amino)-3-methoxy-N-methylbenzamide; |
| 256 | 4-((3-(3-(cyclopropylmethyl)-7-(((3S,4R)-3-fluoro-1-methylpiperidin-4-yl)amino)benzo[b]thiophen-2- |
| | yl)prop-2-yn-1-yl)amino)-3-methoxy-N-methylbenzamide; |
| 257 | 4-((3-(3-(2,2-difluoroethyl)-7-(((3S,4R)-3-fluoro-1-methylpiperidin-4-yl)amino)benzo[b]thiophen-2- |
| | yl)prop-2-yn-1-yl)amino)-3-methoxy-N-methylbenzamide; |
| 258 | 4-((3-(7-(((3S,4R)-3-fluoro-1-methylpiperidin-4-yl)amino)-3-(1H-pyrrol-1-yl)benzo[b]thiophen-2-yl)prop- |
| | 2-yn-1-yl)amino)-3-methoxy-N-methylbenzamide; |
| 259 | 4-((3-(3-ethyl-7-(((3S,4R)-3-fluoro-1-methylpiperidin-4-yl)amino)benzo[b]thiophen-2-yl)prop-2-yn-1- |
| | yl)amino)-3-methoxy-N-methylbenzamide; |
| 260 | 4-((3-(3-(cyanomethyl)-7-((1-methylpiperidin-4-yl)amino)benzo[b]thiophen-2-yl)prop-2-yn-1-yl)amino)-3- |
| | methoxy-N-methylbenzamide; |
| 261 | 3-methoxy-N-methyl-4-((3-(7-((1-methylpiperidin-4-yl)amino)-3-(thiazol-4-yl)benzo[b]thiophen-2-yl)prop- |
| | 2-yn-1-yl)amino)benzamide; |
| 262 | 3-methoxy-N-methyl-4-((3-(7-((1-methylpiperidin-4-yl)amino)-3-(2,2,2-trifluoroethyl)benzo[b]thiophen-2- |
| | yl)prop-2-yn-1-yl)amino)benzamide; |
| 263 | 4-((3-(3-(cyclopropylmethyl)-7-((1-methylpiperidin-4-yl)amino)benzo[b]thiophen-2-yl)prop-2-yn-1- |
| | yl)amino)-3-methoxy-N-methylbenzamide; |
| 264 | 4-((3-(3-(2,2-difluoroethyl)-7-((1-methylpiperidin-4-yl)amino)benzo[b]thiophen-2-yl)prop-2-yn-1- |
| | yl)amino)-3-methoxy-N-methylbenzamide; |
| 265 | 3-methoxy-N-methyl-4-((3-(7-((1-methylpiperidin-4-yl)amino)-3-(1H-pyrrol-1-yl)benzo[b]thiophen-2- |
| | yl)prop-2-yn-1-yl)amino)benzamide; |
| 266 | 4-((3-(3-ethyl-7-((1-methylpiperidin-4-yl)amino)benzo[b]thiophen-2-yl)prop-2-yn-1-yl)amino)-3-methoxy- |
| | N-methylbenzamide; |
| 267 | 4-((3-(3-(cyanomethyl)-7-((tetrahydro-2H-pyran-4-yl)amino)benzo[b]thiophen-2-yl)prop-2-yn-1-yl)amino)- |
| | 3-methoxy-N-methylbenzamide; |
| 268 | 3-methoxy-N-methyl-4-((3-(7-((tetrahydro-2H-pyran-4-yl)amino)-3-(thiazol-4-yl)benzo[b]thiophen-2- |
| | yl)prop-2-yn-1-yl)amino)benzamide; |
| 269 | 3-methoxy-N-methyl-4-((3-(7-((tetrahydro-2H-pyran-4-yl)amino)-3-(2,2,2-trifluoroethyl)benzo[b]thiophen- |
| | 2-yl)prop-2-yn-1-yl)amino)benzamide; |
| 270 | 4-((3-(3-(cyclopropylmethyl)-7-((tetrahydro-2H-pyran-4-yl)amino)benzo[b]thiophen-2-yl)prop-2-yn-1- |
| | yl)amino)-3-methoxy-N-methylbenzamide; |
| 271 | 4-((3-(3-(2,2-difluoroethyl)-7-((tetrahydro-2H-pyran-4-yl)amino)benzo[b]thiophen-2-yl)prop-2-yn-1- |
| | yl)amino)-3-methoxy-N-methylbenzamide; |
| 272 | 4-((3-(3-(1H-pyrrol-1-yl)-7-((tetrahydro-2H-pyran-4-yl)amino)benzo[b]thiophen-2-yl)prop-2-yn-1- |
| | yl)amino)-3-methoxy-N-methylbenzamide; |
| 273 | 4-((3-(3-ethyl-7-((tetrahydro-2H-pyran-4-yl)amino)benzo[b]thiophen-2-yl)prop-2-yn-1-yl)amino)-3- |
| | methoxy-N-methylbenzamide; |
| 274 | 2-(7-((3,3-difluoro-1-methylpiperidin-4-yl)amino)-2-(3-((2-methoxy-4- |
| | (methylsulfonyl)phenyl)amino)prop-1-yn-1-yl)benzo[b]thiophen-3-yl)acetonitrile; |
| 275 | 3,3-difluoro-N-(2-(3-((2-methoxy-4-(methylsulfonyl)phenyl)amino)prop-1-yn-1-yl)-3-(thiazol-4- |
| | yl)benzo[b]thiophen-7-yl)-1-methylpiperidin-4-amine; |
| 276 | 3,3-difluoro-N-(2-(3-((2-methoxy-4-(methylsulfonyl)phenyl)amino)prop-1-yn-1-yl)-3-(2,2,2- |
| | trifluoroethyl)benzo[b]thiophen-7-yl)-1-methylpiperidin-4-amine; |
| 277 | N-(3-(cyclopropylmethyl)-2-(3-((2-methoxy-4-(methylsulfonyl)phenyl)amino)prop-1-yn-1- |
| | yl)benzo[b]thiophen-7-yl)-3,3-difluoro-1-methylpiperidin-4-amine; |
| 278 | N-(3-(2,2-difluoroethyl)-2-(3-((2-methoxy-4-(methylsulfonyl)phenyl)amino)prop-1-yn-1- |
| | yl)benzo[b]thiophen-7-yl)-3,3-difluoro-1-methylpiperidin-4-amine; |
| 279 | 3,3-difluoro-N-(2-(3-((2-methoxy-4-(methylsulfonyl)phenyl)amino)prop-1-yn-1-yl)-3-(1H-pyrrol-1- |
| | yl)benzo[b]thiophen-7-yl)-1-methylpiperidin-4-amine; |
| 280 | N-(3-ethyl-2-(3-((2-methoxy-4-(methylsulfonyl)phenyl)amino)prop-1-yn-1-yl)benzo[b]thiophen-7-yl)-3,3- |
| | difluoro-1-methylpiperidin-4-amine; |
| 281 | 2-(7-((1,1-dioxidotetrahydro-2H-thiopyran-4-yl)amino)-2-(3-((2-methoxy-4- |
| | (methylsulfonyl)phenyl)amino)prop-1-yn-1-yl)benzo[b]thiophen-3-yl)acetonitrile; |
| 282 | 4-((2-(3-((2-methoxy-4-(methylsulfonyl)phenyl)amino)prop-1-yn-1-yl)-3-(thiazol-4-yl)benzo[b]thiophen-7- |
| | yl)amino)tetrahydro-2H-thiopyran 1,1-dioxide; |
| 283 | 4-((2-(3-((2-methoxy-4-(methylsulfonyl)phenyl)amino)prop-1-yn-1-yl)-3-(2,2,2- |
| | trifluoroethyl)benzo[b]thiophen-7-yl)amino)tetrahydro-2H-thiopyran 1,1-dioxide; |
| 284 | 4-((3-(cyclopropylmethyl)-2-(3-((2-methoxy-4-(methylsulfonyl)phenyl)amino)prop-1-yn-1- |
| | yl)benzo[b]thiophen-7-yl)amino)tetrahydro-2H-thiopyran 1,1-dioxide; |
| 285 | 4-((3-(2,2-difluoroethyl)-2-(3-((2-methoxy-4-(methylsulfonyl)phenyl)amino)prop-1-yn-1- |
| | yl)benzo[b]thiophen-7-yl)amino)tetrahydro-2H-thiopyran 1,1-dioxide; |
| 286 | 4-((2-(3-((2-methoxy-4-(methylsulfonyl)phenyl)amino)prop-1-yn-1-yl)-3-(1H-pyrrol-1- |
| | yl)benzo[b]thiophen-7-yl)amino)tetrahydro-2H-thiopyran 1,1-dioxide; |
| 287 | 4-((3-ethyl-2-(3-((2-methoxy-4-(methylsulfonyl)phenyl)amino)prop-1-yn-1-yl)benzo[b]thiophen-7- |
| | yl)amino)tetrahydro-2H-thiopyran 1,1-dioxide; |
| 288 | 2-(7-(((3R,4S)-3-fluoro-1-methylpiperidin-4-yl)amino)-2-(3-((2-methoxy-4- |
| | (methylsulfonyl)phenyl)amino)prop-1-yn-1-yl)benzo[b]thiophen-3-yl)acetonitrile; |
| 289 | (3R,4S)-3-fluoro-N-(2-(3-((2-methoxy-4-(methylsulfonyl)phenyl)amino)prop-1-yn-1-yl)-3-(thiazol-4- |
| | yl)benzo[b]thiophen-7-yl)-1-methylpiperidin-4-amine; |
| 290 | (3R,4S)-3-fluoro-N-(2-(3-((2-methoxy-4-(methylsulfonyl)phenyl)amino)prop-1-yn-1-yl)-3-(2,2,2- |
| | trifluoroethyl)benzo[b]thiophen-7-yl)-1-methylpiperidin-4-amine; |
| 291 | (3R,4S)-N-(3-(cyclopropylmethyl)-2-(3-((2-methoxy-4-(methylsulfonyl)phenyl)amino)prop-1-yn-1- |
| | yl)benzo[b]thiophen-7-yl)-3-fluoro-1-methylpiperidin-4-amine; |
| 292 | (3R,4S)-N-(3-(2,2-difluoroethyl)-2-(3-((2-methoxy-4-(methylsulfonyl)phenyl)amino)prop-1-yn-1- |
| | yl)benzo[b]thiophen-7-yl)-3-fluoro-1-methylpiperidin-4-amine; |
| 293 | (3R,4S)-3-fluoro-N-(2-(3-((2-methoxy-4-(methylsulfonyl)phenyl)amino)prop-1-yn-1-yl)-3-(1H-pyrrol-1- |
| | yl)benzo[b]thiophen-7-yl)-1-methylpiperidin-4-amine; |
| 294 | (3R,4S)-N-(3-ethyl-2-(3-((2-methoxy-4-(methylsulfonyl)phenyl)amino)prop-1-yn-1-yl)benzo[b]thiophen-7- |
| | yl)-3-fluoro-1-methylpiperidin-4-amine; |
| 295 | 2-(7-((3-fluoro-1-methylpiperidin-4-yl)amino)-2-(3-((2-methoxy-4-(methylsulfonyl)phenyl)amino)prop-1- |
| | yn-1-yl)benzo[b]thiophen-3-yl)acetonitrile; |
| 296 | 3-fluoro-N-(2-(3-((2-methoxy-4-(methylsulfonyl)phenyl)amino)prop-1-yn-1-yl)-3-(thiazol-4- |
| | yl)benzo[b]thiophen-7-yl)-1-methylpiperidin-4-amine; |
| 297 | 3-fluoro-N-(2-(3-((2-methoxy-4-(methylsulfonyl)phenyl)amino)prop-1-yn-1-yl)-3-(2,2,2- |
| | trifluoroethyl)benzo[b]thiophen-7-yl)-1-methylpiperidin-4-amine; |
| 298 | N-(3-(cyclopropylmethyl)-2-(3-((2-methoxy-4-(methylsulfonyl)phenyl)amino)prop-1-yn-1- |
| | yl)benzo[b]thiophen-7-yl)-3-fluoro-1-methylpiperidin-4-amine; |
| 299 | N-(3-(2,2-difluoroethyl)-2-(3-((2-methoxy-4-(methylsulfonyl)phenyl)amino)prop-1-yn-1- |
| | yl)benzo[b]thiophen-7-yl)-3-fluoro-1-methylpiperidin-4-amine; |
| 300 | 3-fluoro-N-(2-(3-((2-methoxy-4-(methylsulfonyl)phenyl)amino)prop-1-yn-1-yl)-3-(1H-pyrrol-1- |
| | yl)benzo[b]thiophen-7-yl)-1-methylpiperidin-4-amine; |
| 301 | N-(3-ethyl-2-(3-((2-methoxy-4-(methylsulfonyl)phenyl)amino)prop-1-yn-1-yl)benzo[b]thiophen-7-yl)-3- |
| | fluoro-1-methylpiperidin-4-amine; |
| 302 | 2-(7-((1-(dimethylglycyl)piperidin-4-yl)amino)-2-(3-((2-methoxy-4-(methylsulfonyl)phenyl)amino)prop-1- |
| | yn-1-yl)benzo[b]thiophen-3-yl)acetonitrile; |
| 303 | 2-(dimethylamino)-1-(4-((2-(3-((2-methoxy-4-(methylsulfonyl)phenyl)amino)prop-1-yn-1-yl)-3-(thiazol-4- |
| | yl)benzo[b]thiophen-7-yl)amino)piperidin-1-yl)ethan-1-one; |
| 304 | 2-(dimethylamino)-1-(4-((2-(3-((2-methoxy-4-(methylsulfonyl)phenyl)amino)prop-1-yn-1-yl)-3-(2,2,2- |
| | trifluoroethyl)benzo[b]thiophen-7-yl)amino)piperidin-1-yl)ethan-1-one; |
| 305 | 1-(4-((3-(cyclopropylmethyl)-2-(3-((2-methoxy-4-(methylsulfonyl)phenyl)amino)prop-1-yn-1- |
| | yl)benzo[b]thiophen-7-yl)amino)piperidin-1-yl)-2-(dimethylamino)ethan-1-one; |
| 306 | 1-(4-((3-(2,2-difluoroethyl)-2-(3-((2-methoxy-4-(methylsulfonyl)phenyl)amino)prop-1-yn-1- |
| | yl)benzo[b]thiophen-7-yl)amino)piperidin-1-yl)-2-(dimethylamino)ethan-1-one; |
| 307 | 2-(dimethylamino)-1-(4-((2-(3-((2-methoxy-4-(methylsulfonyl)phenyl)amino)prop-1-yn-1-yl)-3-(1H- |
| | pyrrol-1-yl)benzo[b]thiophen-7-yl)amino)piperidin-1-yl)ethan-1-one; |
| 308 | 2-(dimethylamino)-1-(4-((3-ethyl-2-(3-((2-methoxy-4-(methylsulfonyl)phenyl)amino)prop-1-yn-1- |
| | yl)benzo[b]thiophen-7-yl)amino)piperidin-1-yl)ethan-1-one; |
| 309 | 2-(7-((3-fluoro-1-(2-hydroxy-3-methoxypropyl)piperidin-4-yl)amino)-2-(3-((2-methoxy-4- |
| | (methylsulfonyl)phenyl)amino)prop-1-yn-1-yl)benzo[b]thiophen-3-yl)acetonitrile; |
| 310 | 1-(3-fluoro-4-((2-(3-((2-methoxy-4-(methylsulfonyl)phenyl)amino)prop-1-yn-1-yl)-3-(thiazol-4- |
| | yl)benzo[b]thiophen-7-yl)amino)piperidin-1-yl)-3-methoxypropan-2-ol; |
| 311 | 1-(3-fluoro-4-((2-(3-((2-methoxy-4-(methylsulfonyl)phenyl)amino)prop-1-yn-1-yl)-3-(2,2,2- |
| | trifluoroethyl)benzo[b]thiophen-7-yl)amino)piperidin-1-yl)-3-methoxypropan-2-ol; |
| 312 | 1-(4-((3-(cyclopropylmethyl)-2-(3-((2-methoxy-4-(methylsulfonyl)phenyl)amino)prop-1-yn-1- |
| | yl)benzo[b]thiophen-7-yl)amino)-3-fluoropiperidin-1-yl)-3-methoxypropan-2-o1; |
| 313 | 1-(4-((3-(2,2-difluoroethyl)-2-(3-((2-methoxy-4-(methylsulfonyl)phenyl)amino)prop-1-yn-1- |
| | yl)benzo[b]thiophen-7-yl)amino)-3-fluoropiperidin-1-yl)-3-methoxypropan-2-ol; |
| 314 | 1-(3-fluoro-4-((2-(3-((2-methoxy-4-(methylsulfonyl)phenyl)amino)prop-1-yn-1-yl)-3-(1H-pyrrol-1- |
| | yl)benzo[b]thiophen-7-yl)amino)piperidin-1-yl)-3-methoxypropan-2-o1; |
| 315 | 1-(4-((3-ethyl-2-(3-((2-methoxy-4-(methylsulfonyl)phenyl)amino)prop-1-yn-1-yl)benzo[b]thiophen-7- |
| | yl)amino)-3-fluoropiperidin-1-yl)-3-methoxypropan-2-ol; |
| 316 | 2-(7-(((3R,4R)-3-fluoro-1-methylpiperidin-4-yl)amino)-2-(3-((2-methoxy-4- |
| | (methylsulfonyl)phenyl)amino)prop-1-yn-1-yl)benzo[b]thiophen-3-yl)acetonitrile; |
| 317 | (3R,4R)-3-fluoro-N-(2-(3-((2-methoxy-4-(methylsulfonyl)phenyl)amino)prop-1-yn-1-yl)-3-(thiazol-4- |
| | yl)benzo[b]thiophen-7-yl)-1-methylpiperidin-4-amine; |
| 318 | (3R,4R)-3-fluoro-N-(2-(3-((2-methoxy-4-(methylsulfonyl)phenyl)amino)prop-1-yn-1-yl)-3-(2,2,2- |
| | trifluoroethyl)benzo[b]thiophen-7-yl)-1-methylpiperidin-4-amine; |
| 319 | (3R,4R)-N-(3-(cyclopropylmethyl)-2-(3-((2-methoxy-4-(methylsulfonyl)phenyl)amino)prop-1-yn-1- |
| | yl)benzo[b]thiophen-7-yl)-3-fluoro-1-methylpiperidin-4-amine; |
| 320 | (3R,4R)-N-(3-(2,2-difluoroethyl)-2-(3-((2-methoxy-4-(methylsulfonyl)phenyl)amino)prop-1-yn-1- |
| | yl)benzo[b]thiophen-7-yl)-3-fluoro-1-methylpiperidin-4-amine; |
| 321 | (3R,4R)-3-fluoro-N-(2-(3-((2-methoxy-4-(methylsulfonyl)phenyl)amino)prop-1-yn-1-yl)-3-(1H-pyrrol-1- |
| | yl)benzo[b]thiophen-7-yl)-1-methylpiperidin-4-amine; |
| 322 | (3R,4R)-N-(3-ethyl-2-(3-((2-methoxy-4-(methylsulfonyl)phenyl)amino)prop-1-yn-1-yl)benzo[b]thiophen- |
| | 7-yl)-3-fluoro-1-methylpiperidin-4-amine; |
| 323 | 2-(7-(((3S,4S)-3-fluoro-1-methylpiperidin-4-yl)amino)-2-(3-((2-methoxy-4- |
| | (methylsulfonyl)phenyl)amino)prop-1-yn-1-yl)benzo[b]thiophen-3-yl)acetonitrile; |
| 324 | (3S,4S)-3-fluoro-N-(2-(3-((2-methoxy-4-(methylsulfonyl)phenyl)amino)prop-1-yn-1-yl)-3-(thiazol-4- |
| | yl)benzo[b]thiophen-7-yl)-1-methylpiperidin-4-amine; |
| 325 | (3S,4S)-3-fluoro-N-(2-(3-((2-methoxy-4-(methylsulfonyl)phenyl)amino)prop-1-yn-1-yl)-3-(2,2,2- |
| | trifluoroethyl)benzo[b]thiophen-7-yl)-1-methylpiperidin-4-amine; |
| 326 | (3S,4S)-N-(3-(cyclopropylmethyl)-2-(3-((2-methoxy-4-(methylsulfonyl)phenyl)amino)prop-1-yn-1- |
| | yl)benzo[b]thiophen-7-yl)-3-fluoro-1-methylpiperidin-4-amine; |
| 327 | (3S,4S)-N-(3-(2,2-difluoroethyl)-2-(3-((2-methoxy-4-(methylsulfonyl)phenyl)amino)prop-1-yn-1- |
| | yl)benzo[b]thiophen-7-yl)-3-fluoro-1-methylpiperidin-4-amine; |
| 328 | (3S,4S)-3-fluoro-N-(2-(3-((2-methoxy-4-(methylsulfonyl)phenyl)amino)prop-1-yn-1-yl)-3-(1H-pyrrol-1- |
| | yl)benzo[b]thiophen-7-yl)-1-methylpiperidin-4-amine; |
| 329 | (3S,4S)-N-(3-ethyl-2-(3-((2-methoxy-4-(methylsulfonyl)phenyl)amino)prop-1-yn-1-yl)benzo[b]thiophen-7- |
| | yl)-3-fluoro-1-methylpiperidin-4-amine; |
| 330 | 2-(7-((4-(dimethylamino)cyclohexyl)amino)-2-(3-((2-methoxy-4-(methylsulfonyl)phenyl)amino)prop-1-yn- |
| | 1-yl)benzo[b]thiophen-3-yl)acetonitrile; |
| 331 | N1-(2-(3-((2-methoxy-4-(methylsulfonyl)phenyl)amino)prop-1-yn-1-yl)-3-(thiazol-4-yl)benzo[b]thiophen- |
| | 7-yl)-N4,N4-dimethylcyclohexane-1,4-diamine; |
| 332 | N1-(2-(3-((2-methoxy-4-(methylsulfonyl)phenyl)amino)prop-1-yn-1-yl)-3-(2,2,2- |
| | trifluoroethyl)benzo[b]thiophen-7-yl)-N4,N4-dimethylcyclohexane-1,4-diamine; |
| 333 | N1-(3-(cyclopropylmethyl)-2-(3-((2-methoxy-4-(methylsulfonyl)phenyl)amino)prop-1-yn-1- |
| | yl)benzo[b]thiophen-7-yl)-N4,N4-dimethylcyclohexane-1,4-diamine; |
| 334 | N1-(3-(2,2-difluoroethyl)-2-(3-((2-methoxy-4-(methylsulfonyl)phenyl)amino)prop-1-yn-1- |
| | yl)benzo[b]thiophen-7-yl)-N4,N4-dimethylcyclohexane-1,4-diamine; |
| 335 | N1-(2-(3-((2-methoxy-4-(methylsulfonyl)phenyl)amino)prop-1-yn-1-yl)-3-(1H-pyrrol-1- |
| | yl)benzo[b]thiophen-7-yl)-N4,N4-dimethylcyclohexane-1,4-diamine; |
| 336 | N1-(3-ethyl-2-(3-((2-methoxy-4-(methylsulfonyl)phenyl)amino)prop-1-yn-1-yl)benzo[b]thiophen-7-yl)- |
| | N4,N4-dimethylcyclohexane-1,4-diamine; |
| 337 | 2-(7-((2-(dimethylamino)ethyl)amino)-2-(3-((2-methoxy-4-(methylsulfonyl)phenyl)amino)prop-1-yn-1- |
| | yl)benzo[b]thiophen-3-yl)acetonitrile; |
| 338 | N1-(2-(3-((2-methoxy-4-(methylsulfonyl)phenyl)amino)prop-1-yn-1-yl)-3-(thiazol-4-yl)benzo[b]thiophen- |
| | 7-yl)-N2,N2-dimethylethane-1,2-diamine; |
| 339 | N1-(2-(3-((2-methoxy-4-(methylsulfonyl)phenyl)amino)prop-1-yn-1-yl)-3-(2,2,2- |
| | trifluoroethyl)benzo[b]thiophen-7-yl)-N2,N2-dimethylethane-1,2-diamine; |
| 340 | N1-(3-(cyclopropylmethyl)-2-(3-((2-methoxy-4-(methylsulfonyl)phenyl)amino)prop-1-yn-1- |
| | yl)benzo[b]thiophen-7-yl)-N2,N2-dimethylethane-1,2-diamine; |
| 341 | N1-(3-(2,2-difluoroethyl)-2-(3-((2-methoxy-4-(methylsulfonyl)phenyl)amino)prop-1-yn-1- |
| | yl)benzo[b]thiophen-7-yl)-N2,N2-dimethylethane-1,2-diamine; |
| 342 | N1-(2-(3-((2-methoxy-4-(methylsulfonyl)phenyl)amino)prop-1-yn-1-yl)-3-(1H-pyrrol-1- |
| | yl)benzo[b]thiophen-7-yl)-N2,N2-dimethylethane-1,2-diamine; |
| 343 | N1-(3-ethyl-2-(3-((2-methoxy-4-(methylsulfonyl)phenyl)amino)prop-1-yn-1-yl)benzo[b]thiophen-7-yl)- |
| | N2,N2-dimethylethane-1,2-diamine; |
| 344 | 2-(7-(((3S,4R)-3-fluoro-1-methylpiperidin-4-yl)amino)-2-(3-((2-methoxy-4- |
| | (methylsulfonyl)phenyl)amino)prop-1-yn-1-yl)benzo[b]thiophen-3-yl)acetonitrile; |
| 345 | (3S,4R)-3-fluoro-N-(2-(3-((2-methoxy-4-(methylsulfonyl)phenyl)amino)prop-1-yn-1-yl)-3-(thiazol-4- |
| | yl)benzo[b]thiophen-7-yl)-1-methylpiperidin-4-amine; |
| 346 | (3S,4R)-3-fluoro-N-(2-(3-((2-methoxy-4-(methylsulfonyl)phenyl)amino)prop-1-yn-1-yl)-3-(2,2,2- |
| | trifluoroethyl)benzo[b]thiophen-7-yl)-1-methylpiperidin-4-amine; |
| 347 | (3S,4R)-N-(3-(cyclopropylmethyl)-2-(3-((2-methoxy-4-(methylsulfonyl)phenyl)amino)prop-1-yn-1- |
| | yl)benzo[b]thiophen-7-yl)-3-fluoro-1-methylpiperidin-4-amine; |
| 348 | (3S,4R)-N-(3-(2,2-difluoroethyl)-2-(3-((2-methoxy-4-(methylsulfonyl)phenyl)amino)prop-1-yn-1- |
| | yl)benzo[b]thiophen-7-yl)-3-fluoro-1-methylpiperidin-4-amine; |
| 349 | (3S,4R)-3-fluoro-N-(2-(3-((2-methoxy-4-(methylsulfonyl)phenyl)amino)prop-1-yn-1-yl)-3-(1H-pyrrol-1- |
| | yl)benzo[b]thiophen-7-yl)-1-methylpiperidin-4-amine; |
| 350 | (3S,4R)-N-(3-ethyl-2-(3-((2-methoxy-4-(methylsulfonyl)phenyl)amino)prop-1-yn-1-yl)benzo[b]thiophen-7- |
| | yl)-3-fluoro-1-methylpiperidin-4-amine; |
| 351 | 2-(2-(3-((2-methoxy-4-(methylsulfonyl)phenyl)amino)prop-1-yn-1-yl)-7-((1-methylpiperidin-4- |
| | yl)amino)benzo[b]thiophen-3-yl)acetonitrile; |
| 352 | N-(2-(3-((2-methoxy-4-(methylsulfonyl)phenyl)amino)prop-1-yn-1-yl)-3-(thiazol-4-yl)benzo[b]thiophen-7- |
| | yl)-1-methylpiperidin-4-amine; |
| 353 | N-(2-(3-((2-methoxy-4-(methylsulfonyl)phenyl)amino)prop-1-yn-1-yl)-3-(2,2,2- |
| | trifluoroethyl)benzo[b]thiophen-7-yl)-1-methylpiperidin-4-amine; |
| 354 | N-(3-(cyclopropylmethyl)-2-(3-((2-methoxy-4-(methylsulfonyl)phenyl)amino)prop-1-yn-1- |
| | yl)benzo[b]thiophen-7-yl)-1-methylpiperidin-4-amine; |
| 355 | N-(3-(2,2-difluoroethyl)-2-(3-((2-methoxy-4-(methylsulfonyl)phenyl)amino)prop-1-yn-1- |
| | yl)benzo[b]thiophen-7-yl)-1-methylpiperidin-4-amine; |
| 356 | N-(2-(3-((2-methoxy-4-(methylsulfonyl)phenyl)amino)prop-1-yn-1-yl)-3-(1H-pyrrol-1- |
| | yl)benzo[b]thiophen-7-yl)-1-methylpiperidin-4-amine; |
| 357 | N-(3-ethyl-2-(3-((2-methoxy-4-(methylsulfonyl)phenyl)amino)prop-1-yn-1-yl)benzo[b]thiophen-7-yl)-1- |
| | methylpiperidin-4-amine; |
| 358 | 2-(2-(3-((2-methoxy-4-(methylsulfonyl)phenyl)amino)prop-1-yn-1-yl)-7-((tetrahydro-2H-pyran-4- |
| | yl)amino)benzo[b]thiophen-3-yl)acetonitrile; |
| 359 | N-(2-(3-((2-methoxy-4-(methylsulfonyl)phenyl)amino)prop-1-yn-1-yl)-3-(thiazol-4-yl)benzo[b]thiophen-7- |
| | yl)tetrahydro-2H-pyran-4-amine; |
| 360 | N-(2-(3-((2-methoxy-4-(methylsulfonyl)phenyl)amino)prop-1-yn-1-yl)-3-(2,2,2- |
| | trifluoroethyl)benzo[b]thiophen-7-yl)tetrahydro-2H-pyran-4-amine; |
| 361 | N-(3-(cyclopropylmethyl)-2-(3-((2-methoxy-4-(methylsulfonyl)phenyl)amino)prop-1-yn-1- |
| | yl)benzo[b]thiophen-7-yl)tetrahydro-2H-pyran-4-amine; |
| 362 | N-(3-(2,2-difluoroethyl)-2-(3-((2-methoxy-4-(methylsulfonyl)phenyl)amino)prop-1-yn-1- |
| | yl)benzo[b]thiophen-7-yl)tetrahydro-2H-pyran-4-amine; |
| 363 | N-(2-(3-((2-methoxy-4-(methylsulfonyl)phenyl)amino)prop-1-yn-1-yl)-3-(1H-pyrrol-1- |
| | yl)benzo[b]thiophen-7-yl)tetrahydro-2H-pyran-4-amine; |
| 364 | N-(3-ethyl-2-(3-((2-methoxy-4-(methylsulfonyl)phenyl)amino)prop-1-yn-1-yl)benzo[b]thiophen-7- |
| | yl)tetrahydro-2H-pyran-4-amine; |
| 365 | 4-((3-(3-(cyanomethyl)-7-((3,3-difluoro-1-methylpiperidin-4-yl)amino)benzofuran-2-yl)prop-2-yn-1- |
| | yl)amino)-3-methoxybenzenesulfonamide; |
| 366 | 2-(7-((3,3-difluoro-1-methylpiperidin-4-yl)amino)-2-(3-((5-fluoro-2-methoxy-4- |
| | (methylsulfonyl)phenyl)amino)prop-1-yn-1-yl)benzofuran-3-yl)acetonitrile; |
| 367 | 4-((3-(3-(cyanomethyl)-7-((3,3-difluoro-1-methylpiperidin-4-yl)amino)benzofuran-2-yl)prop-2-yn-1- |
| | yl)amino)-3-methoxy-N-methylbenzamide; |
| 368 | 2-(7-((3,3-difluoro-1-methylpiperidin-4-yl)amino)-2-(3-((2-methoxy-4- |
| | (methylsulfonyl)phenyl)amino)prop-1-yn-1-yl)benzofuran-3-yl)acetonitrile; |
| 369 | 4-((3-(3-(cyanomethyl)-7-((1,1-dioxidotetrahydro-2H-thiopyran-4-yl)amino)benzofuran-2-yl)prop-2-yn-1- |
| | yl)amino)-3-methoxybenzenesulfonamide; |
| 370 | 2-(7-((1,1-dioxidotetrahydro-2H-thiopyran-4-yl)amino)-2-(3-((5-fluoro-2-methoxy-4- |
| | (methylsulfonyl)phenyl)amino)prop-1-yn-1-yl)benzofuran-3-yl)acetonitrile; |
| 371 | 4-((3-(3-(cyanomethyl)-7-((1,1-dioxidotetrahydro-2H-thiopyran-4-yl)amino)benzofuran-2-yl)prop-2-yn-1- |
| | yl)amino)-3-methoxy-N-methylbenzamide; |
| 372 | 2-(7-((1,1-dioxidotetrahydro-2H-thiopyran-4-yl)amino)-2-(3-((2-methoxy-4- |
| | (methylsulfonyl)phenyl)amino)prop-1-yn-1-yl)benzofuran-3-yl)acetonitrile; |
| 373 | 4-((3-(3-(cyanomethyl)-7-(((3R,4S)-3-fluoro-1-methylpiperidin-4-yl)amino)benzofuran-2-yl)prop-2-yn-1- |
| | yl)amino)-3-methoxybenzenesulfonamide; |
| 374 | 2-(7-(((3R,4S)-3-fluoro-1-methylpiperidin-4-yl)amino)-2-(3-((5-fluoro-2-methoxy-4- |
| | (methylsulfonyl)phenyl)amino)prop-1-yn-1-yl)benzofuran-3-yl)acetonitrile; |
| 375 | 4-((3-(3-(cyanomethyl)-7-(((3R,4S)-3-fluoro-1-methylpiperidin-4-yl)amino)benzofuran-2-yl)prop-2-yn-1- |
| | yl)amino)-3-methoxy-N-methylbenzamide; |
| 376 | 2-(7-(((3R,4S)-3-fluoro-1-methylpiperidin-4-yl)amino)-2-(3-((2-methoxy-4- |
| | (methylsulfonyl)phenyl)amino)prop-1-yn-1-yl)benzofuran-3-yl)acetonitrile; |
| 377 | 4-((3-(3-(cyanomethyl)-7-((3-fluoro-1-methylpiperidin-4-yl)amino)benzofuran-2-yl)prop-2-yn-1-yl)amino)- |
| | 3-methoxybenzenesulfonamide; |
| 378 | 2-(7-((3-fluoro-1-methylpiperidin-4-yl)amino)-2-(3-((5-fluoro-2-methoxy-4- |
| | (methylsulfonyl)phenyl)amino)prop-1-yn-1-yl)benzofuran-3-yl)acetonitrile; |
| 379 | 4-((3-(3-(cyanomethyl)-7-((3-fluoro-1-methylpiperidin-4-yl)amino)benzofuran-2-yl)prop-2-yn-1-yl)amino)- |
| | 3-methoxy-N-methylbenzamide; |
| 380 | 2-(7-((3-fluoro-1-methylpiperidin-4-yl)amino)-2-(3-((2-methoxy-4-(methylsulfonyl)phenyl)amino)prop-1- |
| | yn-1-yl)benzofuran-3-yl)acetonitrile; |
| 381 | 4-((3-(3-(cyanomethyl)-7-((1-(dimethylglycyl)piperidin-4-yl)amino)benzofuran-2-yl)prop-2-yn-1- |
| | yl)amino)-3-methoxybenzenesulfonamide; |
| 382 | 2-(7-((1-(dimethylglycyl)piperidin-4-yl)amino)-2-(3-((5-fluoro-2-methoxy-4- |
| | (methylsulfonyl)phenyl)amino)prop-1-yn-1-yl)benzofuran-3-yl)acetonitrile; |
| 383 | 4-((3-(3-(cyanomethyl)-7-((1-(dimethylglycyl)piperidin-4-yl)amino)benzofuran-2-yl)prop-2-yn-1- |
| | yl)amino)-3-methoxy-N-methylbenzamide; |
| 384 | 2-(7-((1-(dimethylglycyl)piperidin-4-yl)amino)-2-(3-((2-methoxy-4-(methylsulfonyl)phenyl)amino)prop-1- |
| | yn-1-yl)benzofuran-3-yl)acetonitrile; |
| 385 | 4-((3-(3-(cyanomethyl)-7-((3-fluoro-1-(2-hydroxy-3-methoxypropyl)piperidin-4-yl)amino)benzofuran-2- |
| | yl)prop-2-yn-1-yl)amino)-3-methoxybenzenesulfonamide; |
| 386 | 2-(7-((3-fluoro-1-(2-hydroxy-3-methoxypropyl)piperidin-4-yl)amino)-2-(3-((5-fluoro-2-methoxy-4- |
| | (methylsulfonyl)phenyl)amino)prop-1-yn-1-yl)benzofuran-3-yl)acetonitrile; |
| 387 | 4-((3-(3-(cyanomethyl)-7-((3-fluoro-1-(2-hydroxy-3-methoxypropyl)piperidin-4-yl)amino)benzofuran-2- |
| | yl)prop-2-yn-1-yl)amino)-3-methoxy-N-methylbenzamide; |
| 388 | 2-(7-((3-fluoro-1-(2-hydroxy-3-methoxypropyl)piperidin-4-yl)amino)-2-(3-((2-methoxy-4- |
| | (methylsulfonyl)phenyl)amino)prop-1-yn-1-yl)benzofuran-3-yl)acetonitrile; |
| 389 | 4-((3-(3-(cyanomethyl)-7-(((3R,4R)-3-fluoro-1-methylpiperidin-4-yl)amino)benzofuran-2-yl)prop-2-yn-1- |
| | yl)amino)-3-methoxybenzenesulfonamide; |
| 390 | 2-(7-(((3R,4R)-3-fluoro-1-methylpiperidin-4-yl)amino)-2-(3-((5-fluoro-2-methoxy-4- |
| | (methylsulfonyl)phenyl)amino)prop-1-yn-1-yl)benzofuran-3-yl)acetonitrile; |
| 391 | 4-((3-(3-(cyanomethyl)-7-(((3R,4R)-3-fluoro-1-methylpiperidin-4-yl)amino)benzofuran-2-yl)prop-2-yn-1- |
| | yl)amino)-3-methoxy-N-methylbenzamide; |
| 392 | 2-(7-(((3R,4R)-3-fluoro-1-methylpiperidin-4-yl)amino)-2-(3-((2-methoxy-4- |
| | (methylsulfonyl)phenyl)amino)prop-1-yn-1-yl)benzofuran-3-yl)acetonitrile; |
| 393 | 4-((3-(3-(cyanomethyl)-7-(((3S,4S)-3-fluoro-1-methylpiperidin-4-yl)amino)benzofuran-2-yl)prop-2-yn-1- |
| | yl)amino)-3-methoxybenzenesulfonamide; |
| 394 | 2-(7-(((3S,4S)-3-fluoro-1-methylpiperidin-4-yl)amino)-2-(3-((5-fluoro-2-methoxy-4- |
| | (methylsulfonyl)phenyl)amino)prop-1-yn-1-yl)benzofuran-3-yl)acetonitrile; |
| 395 | 4-((3-(3-(cyanomethyl)-7-(((3S,4S)-3-fluoro-1-methylpiperidin-4-yl)amino)benzofuran-2-yl)prop-2-yn-1- |
| | yl)amino)-3-methoxy-N-methylbenzamide; |
| 396 | 2-(7-(((3S,4S)-3-fluoro-1-methylpiperidin-4-yl)amino)-2-(3-((2-methoxy-4- |
| | (methylsulfonyl)phenyl)amino)prop-1-yn-1-yl)benzofuran-3-yl)acetonitrile; |
| 397 | 4-((3-(3-(cyanomethyl)-7-((4-(dimethylamino)cyclohexyl)amino)benzofuran-2-yl)prop-2-yn-1-yl)amino)-3- |
| | methoxybenzenesulfonamide; |
| 398 | 2-(7-((4-(dimethylamino)cyclohexyl)amino)-2-(3-((5-fluoro-2-methoxy-4- |
| | (methylsulfonyl)phenyl)amino)prop-1-yn-1-yl)benzofuran-3-yl)acetonitrile; |
| 399 | 4-((3-(3-(cyanomethyl)-7-((4-(dimethylamino)cyclohexyl)amino)benzofuran-2-yl)prop-2-yn-1-yl)amino)-3- |
| | methoxy-N-methylbenzamide; |
| 400 | 2-(7-((4-(dimethylamino)cyclohexyl)amino)-2-(3-((2-methoxy-4-(methylsulfonyl)phenyl)amino)prop-1-yn- |
| | 1-yl)benzofuran-3-yl)acetonitrile; |
| 401 | 4-((3-(3-(cyanomethyl)-7-((2-(dimethylamino)ethyl)amino)benzofuran-2-yl)prop-2-yn-1-yl)amino)-3- |
| | methoxybenzenesulfonamide; |
| 402 | 2-(7-((2-(dimethylamino)ethyl)amino)-2-(3-((5-fluoro-2-methoxy-4-(methylsulfonyl)phenyl)amino)prop-1- |
| | yn-1-yl)benzofuran-3-yl)acetonitrile; |
| 403 | 4-((3-(3-(cyanomethyl)-7-((2-(dimethylamino)ethyl)amino)benzofuran-2-yl)prop-2-yn-1-yl)amino)-3- |
| | methoxy-N-methylbenzamide; |
| 404 | 2-(7-((2-(dimethylamino)ethyl)amino)-2-(3-((2-methoxy-4-(methylsulfonyl)phenyl)amino)prop-1-yn-1- |
| | yl)benzofuran-3-yl)acetonitrile; |
| 405 | 4-((3-(3-(cyanomethyl)-7-(((3S,4R)-3-fluoro-1-methylpiperidin-4-yl)amino)benzofuran-2-yl)prop-2-yn-1- |
| | yl)amino)-3-methoxybenzenesulfonamide; |
| 406 | 2-(7-(((3S,4R)-3-fluoro-1-methylpiperidin-4-yl)amino)-2-(3-((5-fluoro-2-methoxy-4- |
| | (methylsulfonyl)phenyl)amino)prop-1-yn-1-yl)benzofuran-3-yl)acetonitrile; |
| 407 | 4-((3-(3-(cyanomethyl)-7-(((3S,4R)-3-fluoro-1-methylpiperidin-4-yl)amino)benzofuran-2-yl)prop-2-yn-1- |
| | yl)amino)-3-methoxy-N-methylbenzamide; |
| 408 | 2-(7-(((3S,4R)-3-fluoro-1-methylpiperidin-4-yl)amino)-2-(3-((2-methoxy-4- |
| | (methylsulfonyl)phenyl)amino)prop-1-yn-1-yl)benzofuran-3-yl)acetonitrile; |
| 409 | 4-((3-(3-(cyanomethyl)-7-((1-methylpiperidin-4-yl)amino)benzofuran-2-yl)prop-2-yn-1-yl)amino)-3- |
| | methoxybenzenesulfonamide; |
| 410 | 2-(2-(3-((5-fluoro-2-methoxy-4-(methylsulfonyl)phenyl)amino)prop-1-yn-1-yl)-7-((1-methylpiperidin-4- |
| | yl)amino)benzofuran-3-yl)acetonitrile; |
| 411 | 4-((3-(3-(cyanomethyl)-7-((1-methylpiperidin-4-yl)amino)benzofuran-2-yl)prop-2-yn-1-yl)amino)-3- |
| | methoxy-N-methylbenzamide; |
| 412 | 2-(2-(3-((2-methoxy-4-(methylsulfonyl)phenyl)amino)prop-1-yn-1-yl)-7-((1-methylpiperidin-4- |
| | yl)amino)benzofuran-3-yl)acetonitrile; |
| 413 | 4-((3-(3-(cyanomethyl)-7-((tetrahydro-2H-pyran-4-yl)amino)benzofuran-2-yl)prop-2-yn-1-yl)amino)-3- |
| | methoxybenzenesulfonamide; |
| 414 | 2-(2-(3-((5-fluoro-2-methoxy-4-(methylsulfonyl)phenyl)amino)prop-1-yn-1-yl)-7-((tetrahydro-2H-pyran-4- |
| | yl)amino)benzofuran-3-yl)acetonitrile; |
| 415 | 4-((3-(3-(cyanomethyl)-7-((tetrahydro-2H-pyran-4-yl)amino)benzofuran-2-yl)prop-2-yn-1-yl)amino)-3- |
| | methoxy-N-methylbenzamide; |
| 416 | 2-(2-(3-((2-methoxy-4-(methylsulfonyl)phenyl)amino)prop-1-yn-1-yl)-7-((tetrahydro-2H-pyran-4- |
| | yl)amino)benzofuran-3-yl)acetonitrile; |
| 417 | 4-((3-(7-((3,3-difluoro-1-methylpiperidin-4-yl)amino)-3-(thiazol-4-yl)benzofuran-2-yl)prop-2-yn-1- |
| | yl)amino)-3-methoxybenzenesulfonamide; |
| 418 | 3,3-difluoro-N-(2-(3-((5-fluoro-2-methoxy-4-(methylsulfonyl)phenyl)amino)prop-1-yn-1-yl)-3-(thiazol-4- |
| | yl)benzofuran-7-yl)-1-methylpiperidin-4-amine; |
| 419 | 4-((3-(7-((3,3-difluoro-1-methylpiperidin-4-yl)amino)-3-(thiazol-4-yl)benzofuran-2-yl)prop-2-yn-1- |
| | yl)amino)-3-methoxy-N-methylbenzamide; |
| 420 | 3,3-difluoro-N-(2-(3-((2-methoxy-4-(methylsulfonyl)phenyl)amino)prop-1-yn-1-yl)-3-(thiazol-4- |
| | yl)benzofuran-7-yl)-1-methylpiperidin-4-amine; |
| 421 | 4-((3-(7-((1,1-dioxidotetrahydro-2H-thiopyran-4-yl)amino)-3-(thiazol-4-yl)benzofuran-2-yl)prop-2-yn-1- |
| | yl)amino)-3-methoxybenzenesulfonamide; |
| 422 | 4-((2-(3-((5-fluoro-2-methoxy-4-(methylsulfonyl)phenyl)amino)prop-1-yn-1-yl)-3-(thiazol-4- |
| | yl)benzofuran-7-yl)amino)tetrahydro-2H-thiopyran 1,1-dioxide; |
| 423 | 4-((3-(7-((1,1-dioxidotetrahydro-2H-thiopyran-4-yl)amino)-3-(thiazol-4-yl)benzofuran-2-yl)prop-2-yn-1- |
| | yl)amino)-3-methoxy-N-methylbenzamide; |
| 424 | 4-((2-(3-((2-methoxy-4-(methylsulfonyl)phenyl)amino)prop-1-yn-1-yl)-3-(thiazol-4-yl)benzofuran-7- |
| | yl)amino)tetrahydro-2H-thiopyran 1,1-dioxide; |
| 425 | 4-((3-(7-(((3R,4S)-3-fluoro-1-methylpiperidin-4-yl)amino)-3-(thiazol-4-yl)benzofuran-2-yl)prop-2-yn-1- |
| | yl)amino)-3-methoxybenzenesulfonamide; |
| 426 | (3R,4S)-3-fluoro-N-(2-(3-((5-fluoro-2-methoxy-4-(methylsulfonyl)phenyl)amino)prop-1-yn-1-yl)-3- |
| | (thiazol-4-yl)benzofuran-7-yl)-1-methylpiperidin-4-amine; |
| 427 | 4-((3-(7-(((3R,4S)-3-fluoro-1-methylpiperidin-4-yl)amino)-3-(thiazol-4-yl)benzofuran-2-yl)prop-2-yn-1- |
| | yl)amino)-3-methoxy-N-methylbenzamide; |
| 428 | (3R,4S)-3-fluoro-N-(2-(3-((2-methoxy-4-(methylsulfonyl)phenyl)amino)prop-1-yn-1-yl)-3-(thiazol-4- |
| | yl)benzofuran-7-yl)-1-methylpiperidin-4-amine; |
| 429 | 4-((3-(7-((3-fluoro-1-methylpiperidin-4-yl)amino)-3-(thiazol-4-yl)benzofuran-2-yl)prop-2-yn-1-yl)amino)- |
| | 3-methoxybenzenesulfonamide; |
| 430 | 3-fluoro-N-(2-(3-((5-fluoro-2-methoxy-4-(methylsulfonyl)phenyl)amino)prop-1-yn-1-yl)-3-(thiazol-4- |
| | yl)benzofuran-7-yl)-1-methylpiperidin-4-amine; |
| 431 | 4-((3-(7-((3-fluoro-1-methylpiperidin-4-yl)amino)-3-(thiazol-4-yl)benzofuran-2-yl)prop-2-yn-1-yl)amino)- |
| | 3-methoxy-N-methylbenzamide; |
| 432 | 3-fluoro-N-(2-(3-((2-methoxy-4-(methylsulfonyl)phenyl)amino)prop-1-yn-1-yl)-3-(thiazol-4-yl)benzofuran- |
| | 7-yl)-1-methylpiperidin-4-amine; |
| 433 | 4-((3-(7-((1-(dimethylglycyl)piperidin-4-yl)amino)-3-(thiazol-4-yl)benzofuran-2-yl)prop-2-yn-1-yl)amino)- |
| | 3-methoxybenzenesulfonamide; |
| 434 | 2-(dimethylamino)-1-(4-((2-(3-((5-fluoro-2-methoxy-4-(methylsulfonyl)phenyl)amino)prop-1-yn-1-yl)-3- |
| | (thiazol-4-yl)benzofuran-7-yl)amino)piperidin-1-yl)ethan-1-one; |
| 435 | 4-((3-(7-((1-(dimethylglycyl)piperidin-4-yl)amino)-3-(thiazol-4-yl)benzofuran-2-yl)prop-2-yn-1-yl)amino)- |
| | 3-methoxy-N-methylbenzamide; |
| 436 | 2-(dimethylamino)-1-(4-((2-(3-((2-methoxy-4-(methylsulfonyl)phenyl)amino)prop-1-yn-1-yl)-3-(thiazol-4- |
| | yl)benzofuran-7-yl)amino)piperidin-1-yl)ethan-1-one; |
| 437 | 4-((3-(7-((3-fluoro-1-(2-hydroxy-3-methoxypropyl)piperidin-4-yl)amino)-3-(thiazol-4-yl)benzofuran-2- |
| | yl)prop-2-yn-1-yl)amino)-3-methoxybenzenesulfonamide; |
| 438 | 1-(3-fluoro-4-((2-(3-((5-fluoro-2-methoxy-4-(methylsulfonyl)phenyl)amino)prop-1-yn-1-yl)-3-(thiazol-4- |
| | yl)benzofuran-7-yl)amino)piperidin-1-yl)-3-methoxypropan-2-ol; |
| 439 | 4-((3-(7-((3-fluoro-1-(2-hydroxy-3-methoxypropyl)piperidin-4-yl)amino)-3-(thiazol-4-yl)benzofuran-2- |
| | yl)prop-2-yn-1-yl)amino)-3-methoxy-N-methylbenzamide; |
| 440 | 1-(3-fluoro-4-((2-(3-((2-methoxy-4-(methylsulfonyl)phenyl)amino)prop-1-yn-1-yl)-3-(thiazol-4- |
| | yl)benzofuran-7-yl)amino)piperidin-1-yl)-3-methoxypropan-2-o1; |
| 441 | 4-((3-(7-(((3R,4R)-3-fluoro-1-methylpiperidin-4-yl)amino)-3-(thiazol-4-yl)benzofuran-2-yl)prop-2-yn-1- |
| | yl)amino)-3-methoxybenzenesulfonamide; |
| 442 | (3R,4R)-3-fluoro-N-(2-(3-((5-fluoro-2-methoxy-4-(methylsulfonyl)phenyl)amino)prop-1-yn-1-yl)-3- |
| | (thiazol-4-yl)benzofuran-7-yl)-1-methylpiperidin-4-amine; |
| 443 | 4-((3-(7-(((3R,4R)-3-fluoro-1-methylpiperidin-4-yl)amino)-3-(thiazol-4-yl)benzofuran-2-yl)prop-2-yn-1- |
| | yl)amino)-3-methoxy-N-methylbenzamide; |
| 444 | (3R,4R)-3-fluoro-N-(2-(3-((2-methoxy-4-(methylsulfonyl)phenyl)amino)prop-1-yn-1-yl)-3-(thiazol-4- |
| | yl)benzofuran-7-yl)-1-methylpiperidin-4-amine; |
| 445 | 4-((3-(7-(((3S,4S)-3-fluoro-1-methylpiperidin-4-yl)amino)-3-(thiazol-4-yl)benzofuran-2-yl)prop-2-yn-1- |
| | yl)amino)-3-methoxybenzenesulfonamide; |
| 446 | (3S,4S)-3-fluoro-N-(2-(3-((5-fluoro-2-methoxy-4-(methylsulfonyl)phenyl)amino)prop-1-yn-1-yl)-3- |
| | (thiazol-4-yl)benzofuran-7-yl)-1-methylpiperidin-4-amine; |
| 447 | 4-((3-(7-(((3S,4S)-3-fluoro-1-methylpiperidin-4-yl)amino)-3-(thiazol-4-yl)benzofuran-2-yl)prop-2-yn-1- |
| | yl)amino)-3-methoxy-N-methylbenzamide; |
| 448 | (3S,4S)-3-fluoro-N-(2-(3-((2-methoxy-4-(methylsulfonyl)phenyl)amino)prop-1-yn-1-yl)-3-(thiazol-4- |
| | yl)benzofuran-7-yl)-1-methylpiperidin-4-amine; |
| 449 | 4-((3-(7-((4-(dimethylamino)cyclohexyl)amino)-3-(thiazol-4-yl)benzofuran-2-yl)prop-2-yn-1-yl)amino)-3- |
| | methoxybenzenesulfonamide; |
| 450 | N1-(2-(3-((5-fluoro-2-methoxy-4-(methylsulfonyl)phenyl)amino)prop-1-yn-1-yl)-3-(thiazol-4- |
| | yl)benzofuran-7-yl)-N4,N4-dimethylcyclohexane-1,4-diamine; |
| 451 | 4-((3-(7-((4-(dimethylamino)cyclohexyl)amino)-3-(thiazol-4-yl)benzofuran-2-yl)prop-2-yn-1-yl)amino)-3- |
| | methoxy-N-methylbenzamide; |
| 452 | N1-(2-(3-((2-methoxy-4-(methylsulfonyl)phenyl)amino)prop-1-yn-1-yl)-3-(thiazol-4-yl)benzofuran-7-yl)- |
| | N4,N4-dimethylcyclohexane-1,4-diamine; |
| 453 | 4-((3-(7-((2-(dimethylamino)ethyl)amino)-3-(thiazol-4-yl)benzofuran-2-yl)prop-2-yn-1-yl)amino)-3- |
| | methoxybenzenesulfonamide; |
| 454 | N1-(2-(3-((5-fluoro-2-methoxy-4-(methylsulfonyl)phenyl)amino)prop-1-yn-1-yl)-3-(thiazol-4- |
| | yl)benzofuran-7-yl)-N2,N2-dimethylethane-1,2-diamine; |
| 455 | 4-((3-(7-((2-(dimethylamino)ethyl)amino)-3-(thiazol-4-yl)benzofuran-2-yl)prop-2-yn-1-yl)amino)-3- |
| | methoxy-N-methylbenzamide; |
| 456 | N1-(2-(3-((2-methoxy-4-(methylsulfonyl)phenyl)amino)prop-1-yn-1-yl)-3-(thiazol-4-yl)benzofuran-7-yl)- |
| | N2,N2-dimethylethane-1,2-diamine; |
| 457 | 4-((3-(7-(((3S,4R)-3-fluoro-1-methylpiperidin-4-yl)amino)-3-(thiazol-4-yl)benzofuran-2-yl)prop-2-yn-1- |
| | yl)amino)-3-methoxybenzenesulfonamide; |
| 458 | (3S,4R)-3-fluoro-N-(2-(3-((5-fluoro-2-methoxy-4-(methylsulfonyl)phenyl)amino)prop-1-yn-1-yl)-3- |
| | (thiazol-4-yl)benzofuran-7-yl)-1-methylpiperidin-4-amine; |
| 459 | 4-((3-(7-(((3S,4R)-3-fluoro-1-methylpiperidin-4-yl)amino)-3-(thiazol-4-yl)benzofuran-2-yl)prop-2-yn-1- |
| | yl)amino)-3-methoxy-N-methylbenzamide; |
| 460 | (3S,4R)-3-fluoro-N-(2-(3-((2-methoxy-4-(methylsulfonyl)phenyl)amino)prop-1-yn-1-yl)-3-(thiazol-4- |
| | yl)benzofuran-7-yl)-1-methylpiperidin-4-amine; |
| 461 | 3-methoxy-4-((3-(7-((1-methylpiperidin-4-yl)amino)-3-(thiazol-4-yl)benzofuran-2-yl)prop-2-yn-1- |
| | yl)amino)benzenesulfonamide; |
| 462 | N-(2-(3-((5-fluoro-2-methoxy-4-(methylsulfonyl)phenyl)amino)prop-1-yn-1-yl)-3-(thiazol-4-yl)benzofuran- |
| | 7-yl)-1-methylpiperidin-4-amine; |
| 463 | 3-methoxy-N-methyl-4-((3-(7-((1-methylpiperidin-4-yl)amino)-3-(thiazol-4-yl)benzofuran-2-yl)prop-2-yn- |
| | 1-yl)amino)benzamide; |
| 464 | N-(2-(3-((2-methoxy-4-(methylsulfonyl)phenyl)amino)prop-1-yn-1-yl)-3-(thiazol-4-yl)benzofuran-7-yl)-1- |
| | methylpiperidin-4-amine; |
| 465 | 3-methoxy-4-((3-(7-((tetrahydro-2H-pyran-4-yl)amino)-3-(thiazol-4-yl)benzofuran-2-yl)prop-2-yn-1- |
| | yl)amino)benzenesulfonamide; |
| 466 | 2-(3-((5-fluoro-2-methoxy-4-(methylsulfonyl)phenyl)amino)prop-1-yn-1-yl)-N-(tetrahydro-2H-pyran-4-yl)- |
| | 3-(thiazol-4-yl)benzofuran-7-amine; |
| 467 | 3-methoxy-N-methyl-4-((3-(7-((tetrahydro-2H-pyran-4-yl)amino)-3-(thiazol-4-yl)benzofuran-2-yl)prop-2- |
| | yn-1-yl)amino)benzamide; |
| 468 | 2-(3-((2-methoxy-4-(methylsulfonyl)phenyl)amino)prop-1-yn-1-yl)-N-(tetrahydro-2H-pyran-4-yl)-3- |
| | (thiazol-4-yl)benzofuran-7-amine; |
| 469 | 4-((3-(7-((3,3-difluoro-1-methylpiperidin-4-yl)amino)-3-(2,2,2-trifluoroethyl)benzofuran-2-yl)prop-2-yn-1- |
| | yl)amino)-3-methoxybenzenesulfonamide; |
| 470 | 3,3-difluoro-N-(2-(3-((5-fluoro-2-methoxy-4-(methylsulfonyl)phenyl)amino)prop-1-yn-1-yl)-3-(2,2,2- |
| | trifluoroethyl)benzofuran-7-yl)-1-methylpiperidin-4-amine; |
| 471 | 4-((3-(7-((3,3-difluoro-1-methylpiperidin-4-yl)amino)-3-(2,2,2-trifluoroethyl)benzofuran-2-yl)prop-2-yn-1- |
| | yl)amino)-3-methoxy-N-methylbenzamide; |
| 472 | 3,3-difluoro-N-(2-(3-((2-methoxy-4-(methylsulfonyl)phenyl)amino)prop-1-yn-1-yl)-3-(2,2,2- |
| | trifluoroethyl)benzofuran-7-yl)-1-methylpiperidin-4-amine; |
| 473 | 4-((3-(7-((1,1-dioxidotetrahydro-2H-thiopyran-4-yl)amino)-3-(2,2,2-trifluoroethyl)benzofuran-2-yl)prop-2- |
| | yn-1-yl)amino)-3-methoxybenzenesulfonamide; |
| 474 | 4-((2-(3-((5-fluoro-2-methoxy-4-(methylsulfonyl)phenyl)amino)prop-1-yn-1-yl)-3-(2,2,2- |
| | trifluoroethyl)benzofuran-7-yl)amino)tetrahydro-2H-thiopyran 1,1-dioxide; |
| 475 | 4-((3-(7-((1,1-dioxidotetrahydro-2H-thiopyran-4-yl)amino)-3-(2,2,2-trifluoroethyl)benzofuran-2-yl)prop-2- |
| | yn-1-yl)amino)-3-methoxy-N-methylbenzamide; |
| 476 | 4-((2-(3-((2-methoxy-4-(methylsulfonyl)phenyl)amino)prop-1-yn-1-yl)-3-(2,2,2-trifluoroethyl)benzofuran- |
| | 7-yl)amino)tetrahydro-2H-thiopyran 1,1-dioxide; |
| 477 | 4-((3-(7-(((3R,4S)-3-fluoro-1-methylpiperidin-4-yl)amino)-3-(2,2,2-trifluoroethyl)benzofuran-2-yl)prop-2- |
| | yn-1-yl)amino)-3-methoxybenzenesulfonamide; |
| 478 | (3R,4S)-3-fluoro-N-(2-(3-((5-fluoro-2-methoxy-4-(methylsulfonyl)phenyl)amino)prop-1-yn-1-yl)-3-(2,2,2- |
| | trifluoroethyl)benzofuran-7-yl)-1-methylpiperidin-4-amine; |
| 479 | 4-((3-(7-(((3R,4S)-3-fluoro-1-methylpiperidin-4-yl)amino)-3-(2,2,2-trifluoroethyl)benzofuran-2-yl)prop-2- |
| | yn-1-yl)amino)-3-methoxy-N-methylbenzamide; |
| 480 | (3R,4S)-3-fluoro-N-(2-(3-((2-methoxy-4-(methylsulfonyl)phenyl)amino)prop-1-yn-1-yl)-3-(2,2,2- |
| | trifluoroethyl)benzofuran-7-yl)-1-methylpiperidin-4-amine; |
| 481 | 4-((3-(7-((3-fluoro-1-methylpiperidin-4-yl)amino)-3-(2,2,2-trifluoroethyl)benzofuran-2-yl)prop-2-yn-1- |
| | yl)amino)-3-methoxybenzenesulfonamide; |
| 482 | 3-fluoro-N-(2-(3-((5-fluoro-2-methoxy-4-(methylsulfonyl)phenyl)amino)prop-1-yn-1-yl)-3-(2,2,2- |
| | trifluoroethyl)benzofuran-7-yl)-1-methylpiperidin-4-amine; |
| 483 | 4-((3-(7-((3-fluoro-1-methylpiperidin-4-yl)amino)-3-(2,2,2-trifluoroethyl)benzofuran-2-yl)prop-2-yn-1- |
| | yl)amino)-3-methoxy-N-methylbenzamide; |
| 484 | 3-fluoro-N-(2-(3-((2-methoxy-4-(methylsulfonyl)phenyl)amino)prop-1-yn-1-yl)-3-(2,2,2- |
| | trifluoroethyl)benzofuran-7-yl)-1-methylpiperidin-4-amine; |
| 485 | 4-((3-(7-((1-(dimethylglycyl)piperidin-4-yl)amino)-3-(2,2,2-trifluoroethyl)benzofuran-2-yl)prop-2-yn-1- |
| | yl)amino)-3-methoxybenzenesulfonamide; |
| 486 | 2-(dimethylamino)-1-(4-((2-(3-((5-fluoro-2-methoxy-4-(methylsulfonyl)phenyl)amino)prop-1-yn-1-yl)-3- |
| | (2,2,2-trifluoroethyl)benzofuran-7-yl)amino)piperidin-1-yl)ethan-1-one; |
| 487 | 4-((3-(7-((1-(dimethylglycyl)piperidin-4-yl)amino)-3-(2,2,2-trifluoroethyl)benzofuran-2-yl)prop-2-yn-1- |
| | yl)amino)-3-methoxy-N-methylbenzamide; |
| 488 | 2-(dimethylamino)-1-(4-((2-(3-((2-methoxy-4-(methylsulfonyl)phenyl)amino)prop-1-yn-1-yl)-3-(2,2,2- |
| | trifluoroethyl)benzofuran-7-yl)amino)piperidin-1-yl)ethan-1-one; |
| 489 | 4-((3-(7-((3-fluoro-1-(2-hydroxy-3-methoxypropyl)piperidin-4-yl)amino)-3-(2,2,2- |
| | trifluoroethyl)benzofuran-2-yl)prop-2-yn-1-yl)amino)-3-methoxybenzenesulfonamide; |
| 490 | 1-(3-fluoro-4-((2-(3-((5-fluoro-2-methoxy-4-(methylsulfonyl)phenyl)amino)prop-1-yn-1-yl)-3-(2,2,2- |
| | trifluoroethyl)benzofuran-7-yl)amino)piperidin-1-yl)-3-methoxypropan-2-ol; |
| 491 | 4-((3-(7-((3-fluoro-1-(2-hydroxy-3-methoxypropyl)piperidin-4-yl)amino)-3-(2,2,2- |
| | trifluoroethyl)benzofuran-2-yl)prop-2-yn-1-yl)amino)-3-methoxy-N-methylbenzamide; |
| 492 | 1-(3-fluoro-4-((2-(3-((2-methoxy-4-(methylsulfonyl)phenyl)amino)prop-1-yn-1-yl)-3-(2,2,2- |
| | trifluoroethyl)benzofuran-7-yl)amino)piperidin-1-yl)-3-methoxypropan-2-ol; |
| 493 | 4-((3-(7-(((3R,4R)-3-fluoro-1-methylpiperidin-4-yl)amino)-3-(2,2,2-trifluoroethyl)benzofuran-2-yl)prop-2- |
| | yn-1-yl)amino)-3-methoxybenzenesulfonamide; |
| 494 | (3R,4R)-3-fluoro-N-(2-(3-((5-fluoro-2-methoxy-4-(methylsulfonyl)phenyl)amino)prop-1-yn-1-yl)-3-(2,2,2- |
| | trifluoroethyl)benzofuran-7-yl)-1-methylpiperidin-4-amine; |
| 495 | 4-((3-(7-(((3R,4R)-3-fluoro-1-methylpiperidin-4-yl)amino)-3-(2,2,2-trifluoroethyl)benzofuran-2-yl)prop-2- |
| | yn-1-yl)amino)-3-methoxy-N-methylbenzamide; |
| 496 | (3R,4R)-3-fluoro-N-(2-(3-((2-methoxy-4-(methylsulfonyl)phenyl)amino)prop-1-yn-1-yl)-3-(2,2,2- |
| | trifluoroethyl)benzofuran-7-yl)-1-methylpiperidin-4-amine; |
| 497 | 4-((3-(7-(((3S,4S)-3-fluoro-1-methylpiperidin-4-yl)amino)-3-(2,2,2-trifluoroethyl)benzofuran-2-yl)prop-2- |
| | yn-1-yl)amino)-3-methoxybenzenesulfonamide; |
| 498 | (3S,4S)-3-fluoro-N-(2-(3-((5-fluoro-2-methoxy-4-(methylsulfonyl)phenyl)amino)prop-1-yn-1-yl)-3-(2,2,2- |
| | trifluoroethyl)benzofuran-7-yl)-1-methylpiperidin-4-amine; |
| 499 | 4-((3-(7-(((3S,4S)-3-fluoro-1-methylpiperidin-4-yl)amino)-3-(2,2,2-trifluoroethyl)benzofuran-2-yl)prop-2- |
| | yn-1-yl)amino)-3-methoxy-N-methylbenzamide; |
| 500 | (3S,4S)-3-fluoro-N-(2-(3-((2-methoxy-4-(methylsulfonyl)phenyl)amino)prop-1-yn-1-yl)-3-(2,2,2- |
| | trifluoroethyl)benzofuran-7-yl)-1-methylpiperidin-4-amine; |
| 501 | 4-((3-(7-((4-(dimethylamino)cyclohexyl)amino)-3-(2,2,2-trifluoroethyl)benzofuran-2-yl)prop-2-yn-1- |
| | yl)amino)-3-methoxybenzenesulfonamide; |
| 502 | N1-(2-(3-((5-fluoro-2-methoxy-4-(methylsulfonyl)phenyl)amino)prop-1-yn-1-yl)-3-(2,2,2- |
| | trifluoroethyl)benzofuran-7-yl)-N4,N4-dimethylcyclohexane-1,4-diamine; |
| 503 | 4-((3-(7-((4-(dimethylamino)cyclohexyl)amino)-3-(2,2,2-trifluoroethyl)benzofuran-2-yl)prop-2-yn-1- |
| | yl)amino)-3-methoxy-N-methylbenzamide; |
| 504 | N1-(2-(3-((2-methoxy-4-(methylsulfonyl)phenyl)amino)prop-1-yn-1-yl)-3-(2,2,2-trifluoroethyl)benzofuran- |
| | 7-yl)-N4,N4-dimethylcyclohexane-1,4-diamine; |
| 505 | 4-((3-(7-((2-(dimethylamino)ethyl)amino)-3-(2,2,2-trifluoroethyl)benzofuran-2-yl)prop-2-yn-1-yl)amino)- |
| | 3-methoxybenzenesulfonamide; |
| 506 | N1-(2-(3-((5-fluoro-2-methoxy-4-(methylsulfonyl)phenyl)amino)prop-1-yn-1-yl)-3-(2,2,2- |
| | trifluoroethyl)benzofuran-7-yl)-N2,N2-dimethylethane-1,2-diamine; |
| 507 | 4-((3-(7-((2-(dimethylamino)ethyl)amino)-3-(2,2,2-trifluoroethyl)benzofuran-2-yl)prop-2-yn-1-yl)amino)- |
| | 3-methoxy-N-methylbenzamide; |
| 508 | N1-(2-(3-((2-methoxy-4-(methylsulfonyl)phenyl)amino)prop-1-yn-1-yl)-3-(2,2,2-trifluoroethyl)benzofuran- |
| | 7-yl)-N2,N2-dimethylethane-1,2-diamine; |
| 509 | 4-((3-(7-(((3S,4R)-3-fluoro-1-methylpiperidin-4-yl)amino)-3-(2,2,2-trifluoroethyl)benzofuran-2-yl)prop-2- |
| | yn-1-yl)amino)-3-methoxybenzenesulfonamide; |
| 510 | (3S,4R)-3-fluoro-N-(2-(3-((5-fluoro-2-methoxy-4-(methylsulfonyl)phenyl)amino)prop-1-yn-1-yl)-3-(2,2,2- |
| | trifluoroethyl)benzofuran-7-yl)-1-methylpiperidin-4-amine; |
| 511 | 4-((3-(7-(((3S,4R)-3-fluoro-1-methylpiperidin-4-yl)amino)-3-(2,2,2-trifluoroethyl)benzofuran-2-yl)prop-2- |
| | yn-1-yl)amino)-3-methoxy-N-methylbenzamide; |
| 512 | (3S,4R)-3-fluoro-N-(2-(3-((2-methoxy-4-(methylsulfonyl)phenyl)amino)prop-1-yn-1-yl)-3-(2,2,2- |
| | trifluoroethyl)benzofuran-7-yl)-1-methylpiperidin-4-amine; |
| 513 | 3-methoxy-4-((3-(7-((1-methylpiperidin-4-yl)amino)-3-(2,2,2-trifluoroethyl)benzofuran-2-yl)prop-2-yn-1- |
| | yl)amino)benzenesulfonamide; |
| 514 | N-(2-(3-((5-fluoro-2-methoxy-4-(methylsulfonyl)phenyl)amino)prop-1-yn-1-yl)-3-(2,2,2- |
| | trifluoroethyl)benzofuran-7-yl)-1-methylpiperidin-4-amine; |
| 515 | 3-methoxy-N-methyl-4-((3-(7-((1-methylpiperidin-4-yl)amino)-3-(2,2,2-trifluoroethyl)benzofuran-2- |
| | yl)prop-2-yn-1-yl)amino)benzamide; |
| 516 | N-(2-(3-((2-methoxy-4-(methylsulfonyl)phenyl)amino)prop-1-yn-1-yl)-3-(2,2,2-trifluoroethyl)benzofuran- |
| | 7-yl)-1-methylpiperidin-4-amine; |
| 517 | 3-methoxy-4-((3-(7-((tetrahydro-2H-pyran-4-yl)amino)-3-(2,2,2-trifluoroethyl)benzofuran-2-yl)prop-2-yn- |
| | 1-yl)amino)benzenesulfonamide; |
| 518 | 2-(3-((5-fluoro-2-methoxy-4-(methylsulfonyl)phenyl)amino)prop-1-yn-1-yl)-N-(tetrahydro-2H-pyran-4-yl)- |
| | 3-(2,2,2-trifluoroethyl)benzofuran-7-amine; |
| 519 | 3-methoxy-N-methyl-4-((3-(7-((tetrahydro-2H-pyran-4-yl)amino)-3-(2,2,2-trifluoroethyl)benzofuran-2- |
| | yl)prop-2-yn-1-yl)amino)benzamide; |
| 520 | 2-(3-((2-methoxy-4-(methylsulfonyl)phenyl)amino)prop-1-yn-1-yl)-N-(tetrahydro-2H-pyran-4-yl)-3-(2,2,2- |
| | trifluoroethyl)benzofuran-7-amine; |
| 521 | 4-((3-(3-(cyclopropylmethyl)-7-((3,3-difluoro-1-methylpiperidin-4-yl)amino)benzofuran-2-yl)prop-2-yn-1- |
| | yl)amino)-3-methoxybenzenesulfonamide; |
| 522 | N-(3-(cyclopropylmethyl)-2-(3-((5-fluoro-2-methoxy-4-(methylsulfonyl)phenyl)amino)prop-1-yn-1- |
| | yl)benzofuran-7-yl)-3,3-difluoro-1-methylpiperidin-4-amine; |
| 523 | 4-((3-(3-(cyclopropylmethyl)-7-((3,3-difluoro-1-methylpiperidin-4-yl)amino)benzofuran-2-yl)prop-2-yn-1- |
| | yl)amino)-3-methoxy-N-methylbenzamide; |
| 524 | N-(3-(cyclopropylmethyl)-2-(3-((2-methoxy-4-(methylsulfonyl)phenyl)amino)prop-1-yn-1-yl)benzofuran- |
| | 7-yl)-3,3-difluoro-1-methylpiperidin-4-amine; |
| 525 | 4-((3-(3-(cyclopropylmethyl)-7-((1,1-dioxidotetrahydro-2H-thiopyran-4-yl)amino)benzofuran-2-yl)prop-2- |
| | yn-1-yl)amino)-3-methoxybenzenesulfonamide; |
| 526 | 4-((3-(cyclopropylmethyl)-2-(3-((5-fluoro-2-methoxy-4-(methylsulfonyl)phenyl)amino)prop-1-yn-1- |
| | yl)benzofuran-7-yl)amino)tetrahydro-2H-thiopyran 1,1-dioxide; |
| 527 | 4-((3-(3-(cyclopropylmethyl)-7-((1,1-dioxidotetrahydro-2H-thiopyran-4-yl)amino)benzofuran-2-yl)prop-2- |
| | yn-1-yl)amino)-3-methoxy-N-methylbenzamide; |
| 528 | 4-((3-(cyclopropylmethyl)-2-(3-((2-methoxy-4-(methylsulfonyl)phenyl)amino)prop-1-yn-1-yl)benzofuran- |
| | 7-yl)amino)tetrahydro-2H-thiopyran 1,1-dioxide; |
| 529 | 4-((3-(3-(cyclopropylmethyl)-7-(((3R,4S)-3-fluoro-1-methylpiperidin-4-yl)amino)benzofuran-2-yl)prop-2- |
| | yn-1-yl)amino)-3-methoxybenzenesulfonamide; |
| 530 | (3R,4S)-N-(3-(cyclopropylmethyl)-2-(3-((5-fluoro-2-methoxy-4-(methylsulfonyl)phenyl)amino)prop-1-yn- |
| | 1-yl)benzofuran-7-yl)-3-fluoro-1-methylpiperidin-4-amine; |
| 531 | 4-((3-(3-(cyclopropylmethyl)-7-(((3R,4S)-3-fluoro-1-methylpiperidin-4-yl)amino)benzofuran-2-yl)prop-2- |
| | yn-1-yl)amino)-3-methoxy-N-methylbenzamide; |
| 532 | (3R,4S)-N-(3-(cyclopropylmethyl)-2-(3-((2-methoxy-4-(methylsulfonyl)phenyl)amino)prop-1-yn-1- |
| | yl)benzofuran-7-yl)-3-fluoro-1-methylpiperidin-4-amine; |
| 533 | 4-((3-(3-(cyclopropylmethyl)-7-((3-fluoro-1-methylpiperidin-4-yl)amino)benzofuran-2-yl)prop-2-yn-1- |
| | yl)amino)-3-methoxybenzenesulfonamide; |
| 534 | N-(3-(cyclopropylmethyl)-2-(3-((5-fluoro-2-methoxy-4-(methylsulfonyl)phenyl)amino)prop-1-yn-1- |
| | yl)benzofuran-7-yl)-3-fluoro-1-methylpiperidin-4-amine; |
| 535 | 4-((3-(3-(cyclopropylmethyl)-7-((3-fluoro-1-methylpiperidin-4-yl)amino)benzofuran-2-yl)prop-2-yn-1- |
| | yl)amino)-3-methoxy-N-methylbenzamide; |
| 536 | N-(3-(cyclopropylmethyl)-2-(3-((2-methoxy-4-(methylsulfonyl)phenyl)amino)prop-1-yn-1-yl)benzofuran- |
| | 7-yl)-3-fluoro-1-methylpiperidin-4-amine; |
| 537 | 4-((3-(3-(cyclopropylmethyl)-7-((1-(dimethylglycyl)piperidin-4-yl)amino)benzofuran-2-yl)prop-2-yn-1- |
| | yl)amino)-3-methoxybenzenesulfonamide; |
| 538 | 1-(4-((3-(cyclopropylmethyl)-2-(3-((5-fluoro-2-methoxy-4-(methylsulfonyl)phenyl)amino)prop-1-yn-1- |
| | yl)benzofuran-7-yl)amino)piperidin-1-yl)-2-(dimethylamino)ethan-1-one; |
| 539 | 4-((3-(3-(cyclopropylmethyl)-7-((1-(dimethylglycyl)piperidin-4-yl)amino)benzofuran-2-yl)prop-2-yn-1- |
| | yl)amino)-3-methoxy-N-methylbenzamide; |
| 540 | 1-(4-((3-(cyclopropylmethyl)-2-(3-((2-methoxy-4-(methylsulfonyl)phenyl)amino)prop-1-yn-1- |
| | yl)benzofuran-7-yl)amino)piperidin-1-yl)-2-(dimethylamino)ethan-1-one; |
| 541 | 4-((3-(3-(cyclopropylmethyl)-7-((3-fluoro-1-(2-hydroxy-3-methoxypropyl)piperidin-4- |
| | yl)amino)benzofuran-2-yl)prop-2-yn-1-yl)amino)-3-methoxybenzenesulfonamide; |
| 542 | 1-(4-((3-(cyclopropylmethyl)-2-(3-((5-fluoro-2-methoxy-4-(methylsulfonyl)phenyl)amino)prop-1-yn-1- |
| | yl)benzofuran-7-yl)amino)-3-fluoropiperidin-1-yl)-3-methoxypropan-2-ol; |
| 543 | 4-((3-(3-(cyclopropylmethyl)-7-((3-fluoro-1-(2-hydroxy-3-methoxypropyl)piperidin-4- |
| | yl)amino)benzofuran-2-yl)prop-2-yn-1-yl)amino)-3-methoxy-N-methylbenzamide; |
| 544 | 1-(4-((3-(cyclopropylmethyl)-2-(3-((2-methoxy-4-(methylsulfonyl)phenyl)amino)prop-1-yn-1- |
| | yl)benzofuran-7-yl)amino)-3-fluoropiperidin-1-yl)-3-methoxypropan-2-ol; |
| 545 | 4-((3-(3-(cyclopropylmethyl)-7-(((3R,4R)-3-fluoro-1-methylpiperidin-4-yl)amino)benzofuran-2-yl)prop-2- |
| | yn-1-yl)amino)-3-methoxybenzenesulfonamide; |
| 546 | (3R,4R)-N-(3-(cyclopropylmethyl)-2-(3-((5-fluoro-2-methoxy-4-(methylsulfonyl)phenyl)amino)prop-1-yn- |
| | 1-yl)benzofuran-7-yl)-3-fluoro-1-methylpiperidin-4-amine; |
| 547 | 4-((3-(3-(cyclopropylmethyl)-7-(((3R,4R)-3-fluoro-1-methylpiperidin-4-yl)amino)benzofuran-2-yl)prop-2- |
| | yn-1-yl)amino)-3-methoxy-N-methylbenzamide; |
| 548 | (3R,4R)-N-(3-(cyclopropylmethyl)-2-(3-((2-methoxy-4-(methylsulfonyl)phenyl)amino)prop-1-yn-1- |
| | yl)benzofuran-7-yl)-3-fluoro-1-methylpiperidin-4-amine; |
| 549 | 4-((3-(3-(cyclopropylmethyl)-7-(((3S,4S)-3-fluoro-1-methylpiperidin-4-yl)amino)benzofuran-2-yl)prop-2- |
| | yn-1-yl)amino)-3-methoxybenzenesulfonamide; |
| 550 | (3S,4S)-N-(3-(cyclopropylmethyl)-2-(3-((5-fluoro-2-methoxy-4-(methylsulfonyl)phenyl)amino)prop-1-yn- |
| | 1-yl)benzofuran-7-yl)-3-fluoro-1-methylpiperidin-4-amine; |
| 551 | 4-((3-(3-(cyclopropylmethyl)-7-(((3S,4S)-3-fluoro-1-methylpiperidin-4-yl)amino)benzofuran-2-yl)prop-2- |
| | yn-1-yl)amino)-3-methoxy-N-methylbenzamide; |
| 552 | (3S,4S)-N-(3-(cyclopropylmethyl)-2-(3-((2-methoxy-4-(methylsulfonyl)phenyl)amino)prop-1-yn-1- |
| | yl)benzofuran-7-yl)-3-fluoro-1-methylpiperidin-4-amine; |
| 553 | 4-((3-(3-(cyclopropylmethyl)-7-((4-(dimethylamino)cyclohexyl)amino)benzofuran-2-yl)prop-2-yn-1- |
| | yl)amino)-3-methoxybenzenesulfonamide; |
| 554 | N1-(3-(cyclopropylmethyl)-2-(3-((5-fluoro-2-methoxy-4-(methylsulfonyl)phenyl)amino)prop-1-yn-1- |
| | yl)benzofuran-7-yl)-N4,N4-dimethylcyclohexane-1,4-diamine; |
| 555 | 4-((3-(3-(cyclopropylmethyl)-7-((4-(dimethylamino)cyclohexyl)amino)benzofuran-2-yl)prop-2-yn-1- |
| | yl)amino)-3-methoxy-N-methylbenzamide; |
| 556 | N1-(3-(cyclopropylmethyl)-2-(3-((2-methoxy-4-(methylsulfonyl)phenyl)amino)prop-1-yn-1-yl)benzofuran- |
| | 7-yl)-N4,N4-dimethylcyclohexane-1,4-diamine; |
| 557 | 4-((3-(3-(cyclopropylmethyl)-7-((2-(dimethylamino)ethyl)amino)benzofuran-2-yl)prop-2-yn-1-yl)amino)-3- |
| | methoxybenzenesulfonamide; |
| 558 | N1-(3-(cyclopropylmethyl)-2-(3-((5-fluoro-2-methoxy-4-(methylsulfonyl)phenyl)amino)prop-1-yn-1- |
| | yl)benzofuran-7-yl)-N2,N2-dimethylethane-1,2-diamine; |
| 559 | 4-((3-(3-(cyclopropylmethyl)-7-((2-(dimethylamino)ethyl)amino)benzofuran-2-yl)prop-2-yn-1-yl)amino)-3- |
| | methoxy-N-methylbenzamide; |
| 560 | N1-(3-(cyclopropylmethyl)-2-(3-((2-methoxy-4-(methylsulfonyl)phenyl)amino)prop-1-yn-1-yl)benzofuran- |
| | 7-yl)-N2,N2-dimethylethane-1,2-diamine; |
| 561 | 4-((3-(3-(cyclopropylmethyl)-7-(((3S,4R)-3-fluoro-1-methylpiperidin-4-yl)amino)benzofuran-2-yl)prop-2- |
| | yn-1-yl)amino)-3-methoxybenzenesulfonamide; |
| 562 | (3S,4R)-N-(3-(cyclopropylmethyl)-2-(3-((5-fluoro-2-methoxy-4-(methylsulfonyl)phenyl)amino)prop-1-yn- |
| | 1-yl)benzofuran-7-yl)-3-fluoro-1-methylpiperidin-4-amine; |
| 563 | 4-((3-(3-(cyclopropylmethyl)-7-(((3S,4R)-3-fluoro-1-methylpiperidin-4-yl)amino)benzofuran-2-yl)prop-2- |
| | yn-1-yl)amino)-3-methoxy-N-methylbenzamide; |
| 564 | (3S,4R)-N-(3-(cyclopropylmethyl)-2-(3-((2-methoxy-4-(methylsulfonyl)phenyl)amino)prop-1-yn-1- |
| | yl)benzofuran-7-yl)-3-fluoro-1-methylpiperidin-4-amine; |
| 565 | 4-((3-(3-(cyclopropylmethyl)-7-((1-methylpiperidin-4-yl)amino)benzofuran-2-yl)prop-2-yn-1-yl)amino)-3- |
| | methoxybenzenesulfonamide; |
| 566 | N-(3-(cyclopropylmethyl)-2-(3-((5-fluoro-2-methoxy-4-(methylsulfonyl)phenyl)amino)prop-1-yn-1- |
| | yl)benzofuran-7-yl)-1-methylpiperidin-4-amine; |
| 567 | 4-((3-(3-(cyclopropylmethyl)-7-((1-methylpiperidin-4-yl)amino)benzofuran-2-yl)prop-2-yn-1-yl)amino)-3- |
| | methoxy-N-methylbenzamide; |
| 568 | N-(3-(cyclopropylmethyl)-2-(3-((2-methoxy-4-(methylsulfonyl)phenyl)amino)prop-1-yn-1-yl)benzofuran- |
| | 7-yl)-1-methylpiperidin-4-amine; |
| 569 | 4-((3-(3-(cyclopropylmethyl)-7-((tetrahydro-2H-pyran-4-yl)amino)benzofuran-2-yl)prop-2-yn-1-yl)amino)- |
| | 3-methoxybenzenesulfonamide; |
| 570 | 3-(cyclopropylmethyl)-2-(3-((5-fluoro-2-methoxy-4-(methylsulfonyl)phenyl)amino)prop-1-yn-1-yl)-N- |
| | (tetrahydro-2H-pyran-4-yl)benzofuran-7-amine; |
| 571 | 4-((3-(3-(cyclopropylmethyl)-7-((tetrahydro-2H-pyran-4-yl)amino)benzofuran-2-yl)prop-2-yn-1-yl)amino)- |
| | 3-methoxy-N-methylbenzamide; |
| 572 | 3-(cyclopropylmethyl)-2-(3-((2-methoxy-4-(methylsulfonyl)phenyl)amino)prop-1-yn-1-yl)-N-(tetrahydro- |
| | 2H-pyran-4-yl)benzofuran-7-amine; |
| 573 | 4-((3-(7-((3,3-difluoro-1-methylpiperidin-4-yl)amino)-3-(2,2-difluoroethyl)benzofuran-2-yl)prop-2-yn-1- |
| | yl)amino)-3-methoxybenzenesulfonamide; |
| 574 | N-(3-(2,2-difluoroethyl)-2-(3-((5-fluoro-2-methoxy-4-(methylsulfonyl)phenyl)amino)prop-1-yn-1- |
| | yl)benzofuran-7-yl)-3,3-difluoro-1-methylpiperidin-4-amine; |
| 575 | 4-((3-(7-((3,3-difluoro-1-methylpiperidin-4-yl)amino)-3-(2,2-difluoroethyl)benzofuran-2-yl)prop-2-yn-1- |
| | yl)amino)-3-methoxy-N-methylbenzamide; |
| 576 | N-(3-(2,2-difluoroethyl)-2-(3-((2-methoxy-4-(methylsulfonyl)phenyl)amino)prop-1-yn-1-yl)benzofuran-7- |
| | yl)-3,3-difluoro-1-methylpiperidin-4-amine; |
| 577 | 4-((3-(3-(2,2-difluoroethyl)-7-((1,1-dioxidotetrahydro-2H-thiopyran-4-yl)amino)benzofuran-2-yl)prop-2- |
| | yn-1-yl)amino)-3-methoxybenzenesulfonamide; |
| 578 | 4-((3-(2,2-difluoroethyl)-2-(3-((5-fluoro-2-methoxy-4-(methylsulfonyl)phenyl)amino)prop-1-yn-1- |
| | yl)benzofuran-7-yl)amino)tetrahydro-2H-thiopyran 1,1-dioxide; |
| 579 | 4-((3-(3-(2,2-difluoroethyl)-7-((1,1-dioxidotetrahydro-2H-thiopyran-4-yl)amino)benzofuran-2-yl)prop-2- |
| | yn-1-yl)amino)-3-methoxy-N-methylbenzamide; |
| 580 | 4-((3-(2,2-difluoroethyl)-2-(3-((2-methoxy-4-(methylsulfonyl)phenyl)amino)prop-1-yn-1-yl)benzofuran-7- |
| | yl)amino)tetrahydro-2H-thiopyran 1,1-dioxide; |
| 581 | 4-((3-(3-(2,2-difluoroethyl)-7-(((3R,4S)-3-fluoro-1-methylpiperidin-4-yl)amino)benzofuran-2-yl)prop-2-yn- |
| | 1-yl)amino)-3-methoxybenzenesulfonamide; |
| 582 | (3R,4S)-N-(3-(2,2-difluoroethyl)-2-(3-((5-fluoro-2-methoxy-4-(methylsulfonyl)phenyl)amino)prop-1-yn-1- |
| | yl)benzofuran-7-yl)-3-fluoro-1-methylpiperidin-4-amine; |
| 583 | 4-((3-(3-(2,2-difluoroethyl)-7-(((3R,4S)-3-fluoro-1-methylpiperidin-4-yl)amino)benzofuran-2-yl)prop-2-yn- |
| | 1-yl)amino)-3-methoxy-N-methylbenzamide; |
| 584 | (3R,4S)-N-(3-(2,2-difluoroethyl)-2-(3-((2-methoxy-4-(methylsulfonyl)phenyl)amino)prop-1-yn-1- |
| | yl)benzofuran-7-yl)-3-fluoro-1-methylpiperidin-4-amine; |
| 585 | 4-((3-(3-(2,2-difluoroethyl)-7-((3-fluoro-1-methylpiperidin-4-yl)amino)benzofuran-2-yl)prop-2-yn-1- |
| | yl)amino)-3-methoxybenzenesulfonamide; |
| 586 | N-(3-(2,2-difluoroethyl)-2-(3-((5-fluoro-2-methoxy-4-(methylsulfonyl)phenyl)amino)prop-1-yn-1- |
| | yl)benzofuran-7-yl)-3-fluoro-1-methylpiperidin-4-amine; |
| 587 | 4-((3-(3-(2,2-difluoroethyl)-7-((3-fluoro-1-methylpiperidin-4-yl)amino)benzofuran-2-yl)prop-2-yn-1- |
| | yl)amino)-3-methoxy-N-methylbenzamide; |
| 588 | N-(3-(2,2-difluoroethyl)-2-(3-((2-methoxy-4-(methylsulfonyl)phenyl)amino)prop-1-yn-1-yl)benzofuran-7- |
| | yl)-3-fluoro-1-methylpiperidin-4-amine; |
| 589 | 4-((3-(3-(2,2-difluoroethyl)-7-((1-(dimethylglycyl)piperidin-4-yl)amino)benzofuran-2-yl)prop-2-yn-1- |
| | yl)amino)-3-methoxybenzenesulfonamide; |
| 590 | 1-(4-((3-(2,2-difluoroethyl)-2-(3-((5-fluoro-2-methoxy-4-(methylsulfonyl)phenyl)amino)prop-1-yn-1- |
| | yl)benzofuran-7-yl)amino)piperidin-1-yl)-2-(dimethylamino)ethan-1-one; |
| 591 | 4-((3-(3-(2,2-difluoroethyl)-7-((1-(dimethylglycyl)piperidin-4-yl)amino)benzofuran-2-yl)prop-2-yn-1- |
| | yl)amino)-3-methoxy-N-methylbenzamide; |
| 592 | 1-(4-((3-(2,2-difluoroethyl)-2-(3-((2-methoxy-4-(methylsulfonyl)phenyl)amino)prop-1-yn-1-yl)benzofuran- |
| | 7-yl)amino)piperidin-1-yl)-2-(dimethylamino)ethan-1-one; |
| 593 | 4-((3-(3-(2,2-difluoroethyl)-7-((3-fluoro-1-(2-hydroxy-3-methoxypropyl)piperidin-4-yl)amino)benzofuran- |
| | 2-yl)prop-2-yn-1-yl)amino)-3-methoxybenzenesulfonamide; |
| 594 | 1-(4-((3-(2,2-difluoroethyl)-2-(3-((5-fluoro-2-methoxy-4-(methylsulfonyl)phenyl)amino)prop-1-yn-1- |
| | yl)benzofuran-7-yl)amino)-3-fluoropiperidin-1-yl)-3-methoxypropan-2-ol; |
| 595 | 4-((3-(3-(2,2-difluoroethyl)-7-((3-fluoro-1-(2-hydroxy-3-methoxypropyl)piperidin-4-yl)amino)benzofuran- |
| | 2-yl)prop-2-yn-1-yl)amino)-3-methoxy-N-methylbenzamide; |
| 596 | 1-(4-((3-(2,2-difluoroethyl)-2-(3-((2-methoxy-4-(methylsulfonyl)phenyl)amino)prop-1-yn-1-yl)benzofuran- |
| | 7-yl)amino)-3-fluoropiperidin-1-yl)-3-methoxypropan-2-ol; |
| 597 | 4-((3-(3-(2,2-difluoroethyl)-7-(((3R,4R)-3-fluoro-1-methylpiperidin-4-yl)amino)benzofuran-2-yl)prop-2- |
| | yn-1-yl)amino)-3-methoxybenzenesulfonamide; |
| 598 | (3R,4R)-N-(3-(2,2-difluoroethyl)-2-(3-((5-fluoro-2-methoxy-4-(methylsulfonyl)phenyl)amino)prop-1-yn-1- |
| | yl)benzofuran-7-yl)-3-fluoro-1-methylpiperidin-4-amine; |
| 599 | 4-((3-(3-(2,2-difluoroethyl)-7-(((3R,4R)-3-fluoro-1-methylpiperidin-4-yl)amino)benzofuran-2-yl)prop-2- |
| | yn-1-yl)amino)-3-methoxy-N-methylbenzamide; |
| 600 | (3R,4R)-N-(3-(2,2-difluoroethyl)-2-(3-((2-methoxy-4-(methylsulfonyl)phenyl)amino)prop-1-yn-1- |
| | yl)benzofuran-7-yl)-3-fluoro-1-methylpiperidin-4-amine; |
| 601 | 4-((3-(3-(2,2-difluoroethyl)-7-(((3S,4S)-3-fluoro-1-methylpiperidin-4-yl)amino)benzofuran-2-yl)prop-2-yn- |
| | 1-yl)amino)-3-methoxybenzenesulfonamide; |
| 602 | (3S,4S)-N-(3-(2,2-difluoroethyl)-2-(3-((5-fluoro-2-methoxy-4-(methylsulfonyl)phenyl)amino)prop-1-yn-1- |
| | yl)benzofuran-7-yl)-3-fluoro-1-methylpiperidin-4-amine; |
| 603 | 4-((3-(3-(2,2-difluoroethyl)-7-(((3S,4S)-3-fluoro-1-methylpiperidin-4-yl)amino)benzofuran-2-yl)prop-2-yn- |
| | 1-yl)amino)-3-methoxy-N-methylbenzamide; |
| 604 | (3S,4S)-N-(3-(2,2-difluoroethyl)-2-(3-((2-methoxy-4-(methylsulfonyl)phenyl)amino)prop-1-yn-1- |
| | yl)benzofuran-7-yl)-3-fluoro-1-methylpiperidin-4-amine; |
| 605 | 4-((3-(3-(2,2-difluoroethyl)-7-((4-(dimethylamino)cyclohexyl)amino)benzofuran-2-yl)prop-2-yn-1- |
| | yl)amino)-3-methoxybenzenesulfonamide; |
| 606 | N1-(3-(2,2-difluoroethyl)-2-(3-((5-fluoro-2-methoxy-4-(methylsulfonyl)phenyl)amino)prop-1-yn-1- |
| | yl)benzofuran-7-yl)-N4,N4-dimethylcyclohexane-1,4-diamine; |
| 607 | 4-((3-(3-(2,2-difluoroethyl)-7-((4-(dimethylamino)cyclohexyl)amino)benzofuran-2-yl)prop-2-yn-1- |
| | yl)amino)-3-methoxy-N-methylbenzamide; |
| 608 | N1-(3-(2,2-difluoroethyl)-2-(3-((2-methoxy-4-(methylsulfonyl)phenyl)amino)prop-1-yn-1-yl)benzofuran-7- |
| | yl)-N4,N4-dimethylcyclohexane-1,4-diamine; |
| 609 | 4-((3-(3-(2,2-difluoroethyl)-7-((2-(dimethylamino)ethyl)amino)benzofuran-2-yl)prop-2-yn-1-yl)amino)-3- |
| | methoxybenzenesulfonamide; |
| 610 | N1-(3-(2,2-difluoroethyl)-2-(3-((5-fluoro-2-methoxy-4-(methylsulfonyl)phenyl)amino)prop-1-yn-1- |
| | yl)benzofuran-7-yl)-N2,N2-dimethylethane-1,2-diamine; |
| 611 | 4-((3-(3-(2,2-difluoroethyl)-7-((2-(dimethylamino)ethyl)amino)benzofuran-2-yl)prop-2-yn-1-yl)amino)-3- |
| | methoxy-N-methylbenzamide; |
| 612 | N1-(3-(2,2-difluoroethyl)-2-(3-((2-methoxy-4-(methylsulfonyl)phenyl)amino)prop-1-yn-1-yl)benzofuran-7- |
| | yl)-N2,N2-dimethylethane-1,2-diamine; |
| 613 | 4-((3-(3-(2,2-difluoroethyl)-7-(((3S,4R)-3-fluoro-1-methylpiperidin-4-yl)amino)benzofuran-2-yl)prop-2-yn- |
| | 1-yl)amino)-3-methoxybenzenesulfonamide; |
| 614 | (3S,4R)-N-(3-(2,2-difluoroethyl)-2-(3-((5-fluoro-2-methoxy-4-(methylsulfonyl)phenyl)amino)prop-1-yn-1- |
| | yl)benzofuran-7-yl)-3-fluoro-1-methylpiperidin-4-amine; |
| 615 | 4-((3-(3-(2,2-difluoroethyl)-7-(((3S,4R)-3-fluoro-1-methylpiperidin-4-yl)amino)benzofuran-2-yl)prop-2-yn- |
| | 1-yl)amino)-3-methoxy-N-methylbenzamide; |
| 616 | (3S,4R)-N-(3-(2,2-difluoroethyl)-2-(3-((2-methoxy-4-(methylsulfonyl)phenyl)amino)prop-1-yn-1- |
| | yl)benzofuran-7-yl)-3-fluoro-1-methylpiperidin-4-amine; |
| 617 | 4-((3-(3-(2,2-difluoroethyl)-7-((1-methylpiperidin-4-yl)amino)benzofuran-2-yl)prop-2-yn-1-yl)amino)-3- |
| | methoxybenzenesulfonamide; |
| 618 | N-(3-(2,2-difluoroethyl)-2-(3-((5-fluoro-2-methoxy-4-(methylsulfonyl)phenyl)amino)prop-1-yn-1- |
| | yl)benzofuran-7-yl)-1-methylpiperidin-4-amine; |
| 619 | 4-((3-(3-(2,2-difluoroethyl)-7-((1-methylpiperidin-4-yl)amino)benzofuran-2-yl)prop-2-yn-1-yl)amino)-3- |
| | methoxy-N-methylbenzamide; |
| 620 | N-(3-(2,2-difluoroethyl)-2-(3-((2-methoxy-4-(methylsulfonyl)phenyl)amino)prop-1-yn-1-yl)benzofuran-7- |
| | yl)-1-methylpiperidin-4-amine; |
| 621 | 4-((3-(3-(2,2-difluoroethyl)-7-((tetrahydro-2H-pyran-4-yl)amino)benzofuran-2-yl)prop-2-yn-1-yl)amino)-3- |
| | methoxybenzenesulfonamide; |
| 622 | 3-(2,2-difluoroethyl)-2-(3-((5-fluoro-2-methoxy-4-(methylsulfonyl)phenyl)amino)prop-1-yn-1-yl)-N- |
| | (tetrahydro-2H-pyran-4-yl)benzofuran-7-amine; |
| 623 | 4-((3-(3-(2,2-difluoroethyl)-7-((tetrahydro-2H-pyran-4-yl)amino)benzofuran-2-yl)prop-2-yn-1-yl)amino)-3- |
| | methoxy-N-methylbenzamide; |
| 624 | 3-(2,2-difluoroethyl)-2-(3-((2-methoxy-4-(methylsulfonyl)phenyl)amino)prop-1-yn-1-yl)-N-(tetrahydro- |
| | 2H-pyran-4-yl)benzofuran-7-amine; |
| 625 | 4-((3-(7-((3,3-difluoro-1-methylpiperidin-4-yl)amino)-3-(1H-pyrrol-1-yl)benzofuran-2-yl)prop-2-yn-1- |
| | yl)amino)-3-methoxybenzenesulfonamide; |
| 626 | 3,3-difluoro-N-(2-(3-((5-fluoro-2-methoxy-4-(methylsulfonyl)phenyl)amino)prop-1-yn-1-yl)-3-(1H-pyrrol- |
| | 1-yl)benzofuran-7-yl)-1-methylpiperidin-4-amine; |
| 627 | 4-((3-(7-((3,3-difluoro-1-methylpiperidin-4-yl)amino)-3-(1H-pyrrol-1-yl)benzofuran-2-yl)prop-2-yn-1- |
| | yl)amino)-3-methoxy-N-methylbenzamide; |
| 628 | 3,3-difluoro-N-(2-(3-((2-methoxy-4-(methylsulfonyl)phenyl)amino)prop-1-yn-1-yl)-3-(1H-pyrrol-1- |
| | yl)benzofuran-7-yl)-1-methylpiperidin-4-amine; |
| 629 | 4-((3-(7-((1,1-dioxidotetrahydro-2H-thiopyran-4-yl)amino)-3-(1H-pyrrol-1-yl)benzofuran-2-yl)prop-2-yn- |
| | 1-yl)amino)-3-methoxybenzenesulfonamide; |
| 630 | 4-((2-(3-((5-fluoro-2-methoxy-4-(methylsulfonyl)phenyl)amino)prop-1-yn-1-yl)-3-(1H-pyrrol-1- |
| | yl)benzofuran-7-yl)amino)tetrahydro-2H-thiopyran 1,1-dioxide; |
| 631 | 4-((3-(7-((1,1-dioxidotetrahydro-2H-thiopyran-4-yl)amino)-3-(1H-pyrrol-1-yl)benzofuran-2-yl)prop-2-yn- |
| | 1-yl)amino)-3-methoxy-N-methylbenzamide; |
| 632 | 4-((2-(3-((2-methoxy-4-(methylsulfonyl)phenyl)amino)prop-1-yn-1-yl)-3-(1H-pyrrol-1-yl)benzofuran-7- |
| | yl)amino)tetrahydro-2H-thiopyran 1,1-dioxide; |
| 633 | 4-((3-(7-(((3R,4S)-3-fluoro-1-methylpiperidin-4-yl)amino)-3-(1H-pyrrol-1-yl)benzofuran-2-yl)prop-2-yn-1- |
| | yl)amino)-3-methoxybenzenesulfonamide; |
| 634 | (3R,4S)-3-fluoro-N-(2-(3-((5-fluoro-2-methoxy-4-(methylsulfonyl)phenyl)amino)prop-1-yn-1-yl)-3-(1H- |
| | pyrrol-1-yl)benzofuran-7-yl)-1-methylpiperidin-4-amine; |
| 635 | 4-((3-(7-(((3R,4S)-3-fluoro-1-methylpiperidin-4-yl)amino)-3-(1H-pyrrol-1-yl)benzofuran-2-yl)prop-2-yn-1- |
| | yl)amino)-3-methoxy-N-methylbenzamide; |
| 636 | (3R,4S)-3-fluoro-N-(2-(3-((2-methoxy-4-(methylsulfonyl)phenyl)amino)prop-1-yn-1-yl)-3-(1H-pyrrol-1- |
| | yl)benzofuran-7-yl)-1-methylpiperidin-4-amine; |
| 637 | 4-((3-(7-((3-fluoro-1-methylpiperidin-4-yl)amino)-3-(1H-pyrrol-1-yl)benzofuran-2-yl)prop-2-yn-1- |
| | yl)amino)-3-methoxybenzenesulfonamide; |
| 638 | 3-fluoro-N-(2-(3-((5-fluoro-2-methoxy-4-(methylsulfonyl)phenyl)amino)prop-1-yn-1-yl)-3-(1H-pyrrol-1- |
| | yl)benzofuran-7-yl)-1-methylpiperidin-4-amine; |
| 639 | 4-((3-(7-((3-fluoro-1-methylpiperidin-4-yl)amino)-3-(1H-pyrrol-1-yl)benzofuran-2-yl)prop-2-yn-1- |
| | yl)amino)-3-methoxy-N-methylbenzamide; |
| 640 | 3-fluoro-N-(2-(3-((2-methoxy-4-(methylsulfonyl)phenyl)amino)prop-1-yn-1-yl)-3-(1H-pyrrol-1- |
| | yl)benzofuran-7-yl)-1-methylpiperidin-4-amine; |
| 641 | 4-((3-(7-((1-(dimethylglycyl)piperidin-4-yl)amino)-3-(1H-pyrrol-1-yl)benzofuran-2-yl)prop-2-yn-1- |
| | yl)amino)-3-methoxybenzenesulfonamide; |
| 642 | 2-(dimethylamino)-1-(4-((2-(3-((5-fluoro-2-methoxy-4-(methylsulfonyl)phenyl)amino)prop-1-yn-1-yl)-3- |
| | (1H-pyrrol-1-yl)benzofuran-7-yl)amino)piperidin-1-yl)ethan-1-one; |
| 643 | 4-((3-(7-((1-(dimethylglycyl)piperidin-4-yl)amino)-3-(1H-pyrrol-1-yl)benzofuran-2-yl)prop-2-yn-1- |
| | yl)amino)-3-methoxy-N-methylbenzamide; |
| 644 | 2-(dimethylamino)-1-(4-((2-(3-((2-methoxy-4-(methylsulfonyl)phenyl)amino)prop-1-yn-1-yl)-3-(1H- |
| | pyrrol-1-yl)benzofuran-7-yl)amino)piperidin-1-yl)ethan-1-one; |
| 645 | 4-((3-(7-((3-fluoro-1-(2-hydroxy-3-methoxypropyl)piperidin-4-yl)amino)-3-(1H-pyrrol-1-yl)benzofuran-2- |
| | yl)prop-2-yn-1-yl)amino)-3-methoxybenzenesulfonamide; |
| 646 | 1-(3-fluoro-4-((2-(3-((5-fluoro-2-methoxy-4-(methylsulfonyl)phenyl)amino)prop-1-yn-1-yl)-3-(1H-pyrrol- |
| | 1-yl)benzofuran-7-yl)amino)piperidin-1-yl)-3-methoxypropan-2-ol; |
| 647 | 4-((3-(7-((3-fluoro-1-(2-hydroxy-3-methoxypropyl)piperidin-4-yl)amino)-3-(1H-pyrrol-1-yl)benzofuran-2- |
| | yl)prop-2-yn-1-yl)amino)-3-methoxy-N-methylbenzamide; |
| 648 | 1-(3-fluoro-4-((2-(3-((2-methoxy-4-(methylsulfonyl)phenyl)amino)prop-1-yn-1-yl)-3-(1H-pyrrol-1- |
| | yl)benzofuran-7-yl)amino)piperidin-1-yl)-3-methoxypropan-2-ol; |
| 649 | 4-((3-(7-(((3R,4R)-3-fluoro-1-methylpiperidin-4-yl)amino)-3-(1H-pyrrol-1-yl)benzofuran-2-yl)prop-2-yn- |
| | 1-yl)amino)-3-methoxybenzenesulfonamide; |
| 650 | (3R,4R)-3-fluoro-N-(2-(3-((5-fluoro-2-methoxy-4-(methylsulfonyl)phenyl)amino)prop-1-yn-1-yl)-3-(1H- |
| | pyrrol-1-yl)benzofuran-7-yl)-1-methylpiperidin-4-amine; |
| 651 | 4-((3-(7-(((3R,4R)-3-fluoro-1-methylpiperidin-4-yl)amino)-3-(1H-pyrrol-1-yl)benzofuran-2-yl)prop-2-yn- |
| | 1-yl)amino)-3-methoxy-N-methylbenzamide; |
| 652 | (3R,4R)-3-fluoro-N-(2-(3-((2-methoxy-4-(methylsulfonyl)phenyl)amino)prop-1-yn-1-yl)-3-(1H-pyrrol-1- |
| | yl)benzofuran-7-yl)-1-methylpiperidin-4-amine; |
| 653 | 4-((3-(7-(((3S,4S)-3-fluoro-1-methylpiperidin-4-yl)amino)-3-(1H-pyrrol-1-yl)benzofuran-2-yl)prop-2-yn-1- |
| | yl)amino)-3-methoxybenzenesulfonamide; |
| 654 | (3S,4S)-3-fluoro-N-(2-(3-((5-fluoro-2-methoxy-4-(methylsulfonyl)phenyl)amino)prop-1-yn-1-yl)-3-(1H- |
| | pyrrol-1-yl)benzofuran-7-yl)-1-methylpiperidin-4-amine; |
| 655 | 4-((3-(7-(((3S,4S)-3-fluoro-1-methylpiperidin-4-yl)amino)-3-(1H-pyrrol-1-yl)benzofuran-2-yl)prop-2-yn-1- |
| | yl)amino)-3-methoxy-N-methylbenzamide; |
| 656 | (3S,4S)-3-fluoro-N-(2-(3-((2-methoxy-4-(methylsulfonyl)phenyl)amino)prop-1-yn-1-yl)-3-(1H-pyrrol-1- |
| | yl)benzofuran-7-yl)-1-methylpiperidin-4-amine; |
| 657 | 4-((3-(7-((4-(dimethylamino)cyclohexyl)amino)-3-(1H-pyrrol-1-yl)benzofuran-2-yl)prop-2-yn-1-yl)amino)- |
| | 3-methoxybenzenesulfonamide; |
| 658 | N1-(2-(3-((5-fluoro-2-methoxy-4-(methylsulfonyl)phenyl)amino)prop-1-yn-1-yl)-3-(1H-pyrrol-1- |
| | yl)benzofuran-7-yl)-N4,N4-dimethylcyclohexane-1,4-diamine; |
| 659 | 4-((3-(7-((4-(dimethylamino)cyclohexyl)amino)-3-(1H-pyrrol-1-yl)benzofuran-2-yl)prop-2-yn-1-yl)amino)- |
| | 3-methoxy-N-methylbenzamide; |
| 660 | N1-(2-(3-((2-methoxy-4-(methylsulfonyl)phenyl)amino)prop-1-yn-1-yl)-3-(1H-pyrrol-1-yl)benzofuran-7- |
| | yl)-N4,N4-dimethylcyclohexane-1,4-diamine; |
| 661 | 4-((3-(7-((2-(dimethylamino)ethyl)amino)-3-(1H-pyrrol-1-yl)benzofuran-2-yl)prop-2-yn-1-yl)amino)-3- |
| | methoxybenzenesulfonamide; |
| 662 | N1-(2-(3-((5-fluoro-2-methoxy-4-(methylsulfonyl)phenyl)amino)prop-1-yn-1-yl)-3-(1H-pyrrol-1- |
| | yl)benzofuran-7-yl)-N2,N2-dimethylethane-1,2-diamine; |
| 663 | 4-((3-(7-((2-(dimethylamino)ethyl)amino)-3-(1H-pyrrol-1-yl)benzofuran-2-yl)prop-2-yn-1-yl)amino)-3- |
| | methoxy-N-methylbenzamide; |
| 664 | N1-(2-(3-((2-methoxy-4-(methylsulfonyl)phenyl)amino)prop-1-yn-1-yl)-3-(1H-pyrrol-1-yl)benzofuran-7- |
| | yl)-N2,N2-dimethylethane-1,2-diamine; |
| 665 | 4-((3-(7-(((3S,4R)-3-fluoro-1-methylpiperidin-4-yl)amino)-3-(1H-pyrrol-1-yl)benzofuran-2-yl)prop-2-yn-1- |
| | yl)amino)-3-methoxybenzenesulfonamide; |
| 666 | (3S,4R)-3-fluoro-N-(2-(3-((5-fluoro-2-methoxy-4-(methylsulfonyl)phenyl)amino)prop-1-yn-1-yl)-3-(1H- |
| | pyrrol-1-yl)benzofuran-7-yl)-1-methylpiperidin-4-amine; |
| 667 | 4-((3-(7-(((3S,4R)-3-fluoro-1-methylpiperidin-4-yl)amino)-3-(1H-pyrrol-1-yl)benzofuran-2-yl)prop-2-yn-1- |
| | yl)amino)-3-methoxy-N-methylbenzamide; |
| 668 | (3S,4R)-3-fluoro-N-(2-(3-((2-methoxy-4-(methylsulfonyl)phenyl)amino)prop-1-yn-1-yl)-3-(1H-pyrrol-1- |
| | yl)benzofuran-7-yl)-1-methylpiperidin-4-amine; |
| 669 | 3-methoxy-4-((3-(7-((1-methylpiperidin-4-yl)amino)-3-(1H-pyrrol-1-yl)benzofuran-2-yl)prop-2-yn-1- |
| | yl)amino)benzenesulfonamide; |
| 670 | N-(2-(3-((5-fluoro-2-methoxy-4-(methylsulfonyl)phenyl)amino)prop-1-yn-1-yl)-3-(1H-pyrrol-1- |
| | yl)benzofuran-7-yl)-1-methylpiperidin-4-amine; |
| 671 | 3-methoxy-N-methyl-4-((3-(7-((1-methylpiperidin-4-yl)amino)-3-(1H-pyrrol-1-yl)benzofuran-2-yl)prop-2- |
| | yn-1-yl)amino)benzamide; |
| 672 | N-(2-(3-((2-methoxy-4-(methylsulfonyl)phenyl)amino)prop-1-yn-1-yl)-3-(1H-pyrrol-1-yl)benzofuran-7-yl)- |
| | 1-methylpiperidin-4-amine; |
| 673 | 4-((3-(3-(1H-pyrrol-1-yl)-7-((tetrahydro-2H-pyran-4-yl)amino)benzofuran-2-yl)prop-2-yn-1-yl)amino)-3- |
| | methoxybenzenesulfonamide; |
| 674 | 2-(3-((5-fluoro-2-methoxy-4-(methylsulfonyl)phenyl)amino)prop-1-yn-1-yl)-3-(1H-pyrrol-1-yl)-N- |
| | (tetrahydro-2H-pyran-4-yl)benzofuran-7-amine; |
| 675 | 4-((3-(3-(1H-pyrrol-1-yl)-7-((tetrahydro-2H-pyran-4-yl)amino)benzofuran-2-yl)prop-2-yn-1-yl)amino)-3- |
| | methoxy-N-methylbenzamide; |
| 676 | 2-(3-((2-methoxy-4-(methylsulfonyl)phenyl)amino)prop-1-yn-1-yl)-3-(1H-pyrrol-1-yl)-N-(tetrahydro-2H- |
| | pyran-4-yl)benzofuran-7-amine; |
| 677 | 4-((3-(7-((3,3-difluoro-1-methylpiperidin-4-yl)amino)-3-ethylbenzofuran-2-yl)prop-2-yn-1-yl)amino)-3- |
| | methoxybenzenesulfonamide; |
| 678 | N-(3-ethyl-2-(3-((5-fluoro-2-methoxy-4-(methylsulfonyl)phenyl)amino)prop-1-yn-1-yl)benzofuran-7-yl)- |
| | 3,3-difluoro-1-methylpiperidin-4-amine; |
| 679 | 4-((3-(7-((3,3-difluoro-1-methylpiperidin-4-yl)amino)-3-ethylbenzofuran-2-yl)prop-2-yn-1-yl)amino)-3- |
| | methoxy-N-methylbenzamide; |
| 680 | N-(3-ethyl-2-(3-((2-methoxy-4-(methylsulfonyl)phenyl)amino)prop-1-yn-1-yl)benzofuran-7-yl)-3,3- |
| | difluoro-1-methylpiperidin-4-amine; |
| 681 | 4-((3-(7-((1,1-dioxidotetrahydro-2H-thiopyran-4-yl)amino)-3-ethylbenzofuran-2-yl)prop-2-yn-1-yl)amino)- |
| | 3-methoxybenzenesulfonamide; |
| 682 | 4-((3-ethyl-2-(3-((5-fluoro-2-methoxy-4-(methylsulfonyl)phenyl)amino)prop-1-yn-1-yl)benzofuran-7- |
| | yl)amino)tetrahydro-2H-thiopyran 1,1-dioxide; |
| 683 | 4-((3-(7-((1,1-dioxidotetrahydro-2H-thiopyran-4-yl)amino)-3-ethylbenzofuran-2-yl)prop-2-yn-1-yl)amino)- |
| | 3-methoxy-N-methylbenzamide; |
| 684 | 4-((3-ethyl-2-(3-((2-methoxy-4-(methylsulfonyl)phenyl)amino)prop-1-yn-1-yl)benzofuran-7- |
| | yl)amino)tetrahydro-2H-thiopyran 1,1-dioxide; |
| 685 | 4-((3-(3-ethyl-7-(((3R,4S)-3-fluoro-1-methylpiperidin-4-yl)amino)benzofuran-2-yl)prop-2-yn-1-yl)amino)- |
| | 3-methoxybenzenesulfonamide; |
| 686 | (3R,4S)-N-(3-ethyl-2-(3-((5-fluoro-2-methoxy-4-(methylsulfonyl)phenyl)amino)prop-1-yn-1- |
| | yl)benzofuran-7-yl)-3-fluoro-1-methylpiperidin-4-amine; |
| 687 | 4-((3-(3-ethyl-7-(((3R,4S)-3-fluoro-1-methylpiperidin-4-yl)amino)benzofuran-2-yl)prop-2-yn-1-yl)amino)- |
| | 3-methoxy-N-methylbenzamide; |
| 688 | (3R,4S)-N-(3-ethyl-2-(3-((2-methoxy-4-(methylsulfonyl)phenyl)amino)prop-1-yn-1-yl)benzofuran-7-yl)-3- |
| | fluoro-1-methylpiperidin-4-amine; |
| 689 | 4-((3-(3-ethyl-7-((3-fluoro-1-methylpiperidin-4-yl)amino)benzofuran-2-yl)prop-2-yn-1-yl)amino)-3- |
| | methoxybenzenesulfonamide; |
| 690 | N-(3-ethyl-2-(3-((5-fluoro-2-methoxy-4-(methylsulfonyl)phenyl)amino)prop-1-yn-1-yl)benzofuran-7-yl)-3- |
| | fluoro-1-methylpiperidin-4-amine; |
| 691 | 4-((3-(3-ethyl-7-((3-fluoro-1-methylpiperidin-4-yl)amino)benzofuran-2-yl)prop-2-yn-1-yl)amino)-3- |
| | methoxy-N-methylbenzamide; |
| 692 | N-(3-ethyl-2-(3-((2-methoxy-4-(methylsulfonyl)phenyl)amino)prop-1-yn-1-yl)benzofuran-7-yl)-3-fluoro-1- |
| | methylpiperidin-4-amine; |
| 693 | 4-((3-(7-((1-(dimethylglycyl)piperidin-4-yl)amino)-3-ethylbenzofuran-2-yl)prop-2-yn-1-yl)amino)-3- |
| | methoxybenzenesulfonamide; |
| 694 | 2-(dimethylamino)-1-(4-((3-ethyl-2-(3-((5-fluoro-2-methoxy-4-(methylsulfonyl)phenyl)amino)prop-1-yn-1- |
| | yl)benzofuran-7-yl)amino)piperidin-1-yl)ethan-1-one; |
| 695 | 4-((3-(7-((1-(dimethylglycyl)piperidin-4-yl)amino)-3-ethylbenzofuran-2-yl)prop-2-yn-1-yl)amino)-3- |
| | methoxy-N-methylbenzamide; |
| 696 | 2-(dimethylamino)-1-(4-((3-ethyl-2-(3-((2-methoxy-4-(methylsulfonyl)phenyl)amino)prop-1-yn-1- |
| | yl)benzofuran-7-yl)amino)piperidin-1-yl)ethan-1-one; |
| 697 | 4-((3-(3-ethyl-7-((3-fluoro-1-(2-hydroxy-3-methoxypropyl)piperidin-4-yl)amino)benzofuran-2-yl)prop-2- |
| | yn-1-yl)amino)-3-methoxybenzenesulfonamide; |
| 698 | 1-(4-((3-ethyl-2-(3-((5-fluoro-2-methoxy-4-(methylsulfonyl)phenyl)amino)prop-1-yn-1-yl)benzofuran-7- |
| | yl)amino)-3-fluoropiperidin-1-yl)-3-methoxypropan-2-o1; |
| 699 | 4-((3-(3-ethyl-7-((3-fluoro-1-(2-hydroxy-3-methoxypropyl)piperidin-4-yl)amino)benzofuran-2-yl)prop-2- |
| | yn-1-yl)amino)-3-methoxy-N-methylbenzamide; |
| 700 | 1-(4-((3-ethyl-2-(3-((2-methoxy-4-(methylsulfonyl)phenyl)amino)prop-1-yn-1-yl)benzofuran-7-yl)amino)- |
| | 3-fluoropiperidin-1-yl)-3-methoxypropan-2-ol; |
| 701 | 4-((3-(3-ethyl-7-(((3R,4R)-3-fluoro-1-methylpiperidin-4-yl)amino)benzofuran-2-yl)prop-2-yn-1-yl)amino)- |
| | 3-methoxybenzenesulfonamide; |
| 702 | (3R,4R)-N-(3-ethyl-2-(3-((5-fluoro-2-methoxy-4-(methylsulfonyl)phenyl)amino)prop-1-yn-1- |
| | yl)benzofuran-7-yl)-3-fluoro-1-methylpiperidin-4-amine; |
| 703 | 4-((3-(3-ethyl-7-(((3R,4R)-3-fluoro-1-methylpiperidin-4-yl)amino)benzofuran-2-yl)prop-2-yn-1-yl)amino)- |
| | 3-methoxy-N-methylbenzamide; |
| 704 | (3R,4R)-N-(3-ethyl-2-(3-((2-methoxy-4-(methylsulfonyl)phenyl)amino)prop-1-yn-1-yl)benzofuran-7-yl)-3- |
| | fluoro-1-methylpiperidin-4-amine; |
| 705 | 4-((3-(3-ethyl-7-(((3S,4S)-3-fluoro-1-methylpiperidin-4-yl)amino)benzofuran-2-yl)prop-2-yn-1-yl)amino)- |
| | 3-methoxybenzenesulfonamide; |
| 706 | (3S,4S)-N-(3-ethyl-2-(3-((5-fluoro-2-methoxy-4-(methylsulfonyl)phenyl)amino)prop-1-yn-1- |
| | yl)benzofuran-7-yl)-3-fluoro-1-methylpiperidin-4-amine; |
| 707 | 4-((3-(3-ethyl-7-(((3S,4S)-3-fluoro-1-methylpiperidin-4-yl)amino)benzofuran-2-yl)prop-2-yn-1-yl)amino)- |
| | 3-methoxy-N-methylbenzamide; |
| 708 | (3S,4S)-N-(3-ethyl-2-(3-((2-methoxy-4-(methylsulfonyl)phenyl)amino)prop-1-yn-1-yl)benzofuran-7-yl)-3- |
| | fluoro-1-methylpiperidin-4-amine; |
| 709 | 4-((3-(7-((4-(dimethylamino)cyclohexyl)amino)-3-ethylbenzofuran-2-yl)prop-2-yn-1-yl)amino)-3- |
| | methoxybenzenesulfonamide; |
| 710 | N1-(3-ethyl-2-(3-((5-fluoro-2-methoxy-4-(methylsulfonyl)phenyl)amino)prop-1-yn-1-yl)benzofuran-7-yl)- |
| | N4,N4-dimethylcyclohexane-1,4-diamine; |
| 711 | 4-((3-(7-((4-(dimethylamino)cyclohexyl)amino)-3-ethylbenzofuran-2-yl)prop-2-yn-1-yl)amino)-3-methoxy- |
| | N-methylbenzamide; |
| 712 | N1-(3-ethyl-2-(3-((2-methoxy-4-(methylsulfonyl)phenyl)amino)prop-1-yn-1-yl)benzofuran-7-yl)-N4, N4- |
| | dimethylcyclohexane-1,4-diamine; |
| 713 | 4-((3-(7-((2-(dimethylamino)ethyl)amino)-3-ethylbenzofuran-2-yl)prop-2-yn-1-yl)amino)-3- |
| | methoxybenzenesulfonamide; |
| 714 | N1-(3-ethyl-2-(3-((5-fluoro-2-methoxy-4-(methylsulfonyl)phenyl)amino)prop-1-yn-1-yl)benzofuran-7-yl)- |
| | N2,N2-dimethylethane-1,2-diamine; |
| 715 | 4-((3-(7-((2-(dimethylamino)ethyl)amino)-3-ethylbenzofuran-2-yl)prop-2-yn-1-yl)amino)-3-methoxy-N- |
| | methylbenzamide; |
| 716 | N1-(3-ethyl-2-(3-((2-methoxy-4-(methylsulfonyl)phenyl)amino)prop-1-yn-1-yl)benzofuran-7-yl)-N2,N2- |
| | dimethylethane-1,2-diamine; |
| 717 | 4-((3-(3-ethyl-7-(((3S,4R)-3-fluoro-1-methylpiperidin-4-yl)amino)benzofuran-2-yl)prop-2-yn-1-yl)amino)- |
| | 3-methoxybenzenesulfonamide; |
| 718 | (3S,4R)-N-(3-ethyl-2-(3-((5-fluoro-2-methoxy-4-(methylsulfonyl)phenyl)amino)prop-1-yn-1- |
| | yl)benzofuran-7-yl)-3-fluoro-1-methylpiperidin-4-amine; |
| 719 | 4-((3-(3-ethyl-7-(((3S,4R)-3-fluoro-1-methylpiperidin-4-yl)amino)benzofuran-2-yl)prop-2-yn-1-yl)amino)- |
| | 3-methoxy-N-methylbenzamide; |
| 720 | (3S,4R)-N-(3-ethyl-2-(3-((2-methoxy-4-(methylsulfonyl)phenyl)amino)prop-1-yn-1-yl)benzofuran-7-yl)-3- |
| | fluoro-1-methylpiperidin-4-amine; |
| 721 | 4-((3-(3-ethyl-7-((1-methylpiperidin-4-yl)amino)benzofuran-2-yl)prop-2-yn-1-yl)amino)-3- |
| | methoxybenzenesulfonamide; |
| 722 | N-(3-ethyl-2-(3-((5-fluoro-2-methoxy-4-(methylsulfonyl)phenyl)amino)prop-1-yn-1-yl)benzofuran-7-yl)-1- |
| | methylpiperidin-4-amine; |
| 723 | 4-((3-(3-ethyl-7-((1-methylpiperidin-4-yl)amino)benzofuran-2-yl)prop-2-yn-1-yl)amino)-3-methoxy-N- |
| | methylbenzamide; |
| 724 | N-(3-ethyl-2-(3-((2-methoxy-4-(methylsulfonyl)phenyl)amino)prop-1-yn-1-yl)benzofuran-7-yl)-1- |
| | methylpiperidin-4-amine; |
| 725 | 4-((3-(3-ethyl-7-((tetrahydro-2H-pyran-4-yl)amino)benzofuran-2-yl)prop-2-yn-1-yl)amino)-3- |
| | methoxybenzenesulfonamide; |
| 726 | 3-ethyl-2-(3-((5-fluoro-2-methoxy-4-(methylsulfonyl)phenyl)amino)prop-1-yn-1-yl)-N-(tetrahydro-2H- |
| | pyran-4-yl)benzofuran-7-amine; |
| 727 | 4-((3-(3-ethyl-7-((tetrahydro-2H-pyran-4-yl)amino)benzofuran-2-yl)prop-2-yn-1-yl)amino)-3-methoxy-N- |
| | methylbenzamide; |
| 728 | 3-ethyl-2-(3-((2-methoxy-4-(methylsulfonyl)phenyl)amino)prop-1-yn-1-yl)-N-(tetrahydro-2H-pyran-4- |
| | yl)benzofuran-7-amine; |
| 729 | 3-(cyclopropylmethyl)-2-(3-((2-methoxy-4-(methylsulfonyl)phenyl)amino)prop-1-yn-1-yl)-N-(1- |
| | methylpiperidin-4-yl)thieno[3,2-b]pyridin-7-amine; |
| 730 | 2-(3-((2-methoxy-4-(methylsulfonyl)phenyl)amino)prop-1-yn-1-yl)-N-(1-methylpiperidin-4-yl)-3-(2,2,2- |
| | trifluoroethyl)thieno[3,2-b]pyridin-7-amine; |
| 731 | 3-ethyl-2-(3-((2-methoxy-4-(methylsulfonyl)phenyl)amino)prop-1-yn-1-yl)-N-(1-methylpiperidin-4- |
| | yl)thieno[3,2-b]pyridin-7-amine; |
| 732 | 4-((3-(3-(cyclopropylmethyl)-7-((1-methylpiperidin-4-yl)amino)thieno[3,2-b]pyridin-2-yl)prop-2-yn-1- |
| | yl)amino)-3-methoxy-N-methylbenzamide; |
| 733 | 3-methoxy-N-methyl-4-((3-(7-((1-methylpiperidin-4-yl)amino)-3-(2,2,2-trifluoroethyl)thieno[3,2-b]pyridin- |
| | 2-yl)prop-2-yn-1-yl)amino)benzamide; |
| 734 | 4-((3-(3-ethyl-7-((1-methylpiperidin-4-yl)amino)thieno[3,2-b]pyridin-2-yl)prop-2-yn-1-yl)amino)-3- |
| | methoxy-N-methylbenzamide; |
| 735 | 4-((3-(3-(cyclopropylmethyl)-7-((1-methylpiperidin-4-yl)amino)thieno[3,2-b]pyridin-2-yl)prop-2-yn-1- |
| | yl)amino)-3-methoxybenzenesulfonamide; |
| 736 | 3-methoxy-4-((3-(7-((1-methylpiperidin-4-yl)amino)-3-(2,2,2-trifluoroethyl)thieno[3,2-b]pyridin-2-yl)prop- |
| | 2-yn-1-yl)amino)benzenesulfonamide; |
| 737 | 4-((3-(3-ethyl-7-((1-methylpiperidin-4-yl)amino)thieno[3,2-b]pyridin-2-yl)prop-2-yn-1-yl)amino)-3- |
| | methoxybenzenesulfonamide; |
| 738 | 1-(4-((3-(cyclopropylmethyl)-2-(3-((2-methoxy-4-(methylsulfonyl)phenyl)amino)prop-1-yn-1- |
| | yl)thieno[3,2-b]pyridin-7-yl)amino)-3-fluoropiperidin-1-yl)-3-methoxypropan-2-ol; |
| 739 | 1-(3-fluoro-4-((2-(3-((2-methoxy-4-(methylsulfonyl)phenyl)amino)prop-1-yn-1-yl)-3-(2,2,2- |
| | trifluoroethyl)thieno[3,2-b]pyridin-7-yl)amino)piperidin-1-yl)-3-methoxypropan-2-ol; |
| 740 | 1-(4-((3-ethyl-2-(3-((2-methoxy-4-(methylsulfonyl)phenyl)amino)prop-1-yn-1-yl)thieno[3,2-b]pyridin-7- |
| | yl)amino)-3-fluoropiperidin-1-yl)-3-methoxypropan-2-ol; |
| 741 | 4-((3-(3-(cyclopropylmethyl)-7-((3-fluoro-1-(2-hydroxy-3-methoxypropyl)piperidin-4-yl)amino)thieno [3,2- |
| | b]pyridin-2-yl)prop-2-yn-1-yl)amino)-3-methoxy-N-methylbenzamide; |
| 742 | 4-((3-(7-((3-fluoro-1-(2-hydroxy-3-methoxypropyl)piperidin-4-yl)amino)-3-(2,2,2-trifluoroethyl)thieno [3,2- |
| | b]pyridin-2-yl)prop-2-yn-1-yl)amino)-3-methoxy-N-methylbenzamide; |
| 743 | 4-((3-(3-ethyl-7-((3-fluoro-1-(2-hydroxy-3-methoxypropyl)piperidin-4-yl)amino)thieno[3,2-b]pyridin-2- |
| | yl)prop-2-yn-1-yl)amino)-3-methoxy-N-methylbenzamide; |
| 744 | 4-((3-(3-(cyclopropylmethyl)-7-((3-fluoro-1-(2-hydroxy-3-methoxypropyl)piperidin-4-yl)amino)thieno[3,2- |
| | b]pyridin-2-yl)prop-2-yn-1-yl)amino)-3-methoxybenzenesulfonamide; |
| 745 | 4-((3-(7-((3-fluoro-1-(2-hydroxy-3-methoxypropyl)piperidin-4-yl)amino)-3-(2,2,2-trifluoroethyl)thieno[3,2- |
| | b]pyridin-2-yl)prop-2-yn-1-yl)amino)-3-methoxybenzenesulfonamide; |
| 746 | 4-((3-(3-ethyl-7-((3-fluoro-1-(2-hydroxy-3-methoxypropyl)piperidin-4-yl)amino)thieno[3,2-b]pyridin-2- |
| | yl)prop-2-yn-1-yl)amino)-3-methoxybenzenesulfonamide; |
| 747 | 3-(cyclopropylmethyl)-N-(3-fluoro-1-methylpiperidin-4-yl)-2-(3-((2-methoxy-4- |
| | (methylsulfonyl)phenyl)amino)prop-1-yn-1-yl)thieno[3,2-b]pyridin-7-amine; |
| 748 | N-(3-fluoro-1-methylpiperidin-4-yl)-2-(3-((2-methoxy-4-(methylsulfonyl)phenyl)amino)prop-1-yn-1-yl)-3- |
| | (2,2,2-trifluoroethyl)thieno[3,2-b]pyridin-7-amine; |
| 749 | 3-ethyl-N-(3-fluoro-1-methylpiperidin-4-yl)-2-(3-((2-methoxy-4-(methylsulfonyl)phenyl)amino)prop-1-yn- |
| | 1-yl)thieno[3,2-b]pyridin-7-amine; |
| 750 | 4-((3-(3-(cyclopropylmethyl)-7-((3-fluoro-1-methylpiperidin-4-yl)amino)thieno[3,2-b]pyridin-2-yl)prop-2- |
| | yn-1-yl)amino)-3-methoxy-N-methylbenzamide; |
| 751 | 4-((3-(7-((3-fluoro-1-methylpiperidin-4-yl)amino)-3-(2,2,2-trifluoroethyl)thieno[3,2-b]pyridin-2-yl)prop-2- |
| | yn-1-yl)amino)-3-methoxy-N-methylbenzamide; |
| 752 | 4-((3-(3-ethyl-7-((3-fluoro-1-methylpiperidin-4-yl)amino)thieno[3,2-b]pyridin-2-yl)prop-2-yn-1-yl)amino)- |
| | 3-methoxy-N-methylbenzamide; |
| 753 | 4-((3-(3-(cyclopropylmethyl)-7-((3-fluoro-1-methylpiperidin-4-yl)amino)thieno[3,2-b]pyridin-2-yl)prop-2- |
| | yn-1-yl)amino)-3-methoxybenzenesulfonamide; |
| 754 | 4-((3-(7-((3-fluoro-1-methylpiperidin-4-yl)amino)-3-(2,2,2-trifluoroethyl)thieno[3,2-b]pyridin-2-yl)prop-2- |
| | yn-1-yl)amino)-3-methoxybenzenesulfonamide; |
| 755 | 4-((3-(3-ethyl-7-((3-fluoro-1-methylpiperidin-4-yl)amino)thieno[3,2-b]pyridin-2-yl)prop-2-yn-1-yl)amino)- |
| | 3-methoxybenzenesulfonamide; |
| 756 | N1-(3-(cyclopropylmethyl)-2-(3-((2-methoxy-4-(methylsulfonyl)phenyl)amino)prop-1-yn-1-yl)thieno[3,2- |
| | b]pyridin-7-yl)-N4,N4-dimethylcyclohexane-1,4-diamine; |
| 757 | N1-(2-(3-((2-methoxy-4-(methylsulfonyl)phenyl)amino)prop-1-yn-1-yl)-3-(2,2,2-trifluoroethyl)thieno[3,2- |
| | b]pyridin-7-yl)-N4,N4-dimethylcyclohexane-1,4-diamine; |
| 758 | N1-(3-ethyl-2-(3-((2-methoxy-4-(methylsulfonyl)phenyl)amino)prop-1-yn-1-yl)thieno[3,2-b]pyridin-7-yl)- |
| | N4,N4-dimethylcyclohexane-1,4-diamine; |
| 759 | 4-((3-(3-(cyclopropylmethyl)-7-((4-(dimethylamino)cyclohexyl)amino)thieno[3,2-b]pyridin-2-yl)prop-2- |
| | yn-1-yl)amino)-3-methoxy-N-methylbenzamide; |
| 760 | 4-((3-(7-((4-(dimethylamino)cyclohexyl)amino)-3-(2,2,2-trifluoroethyl)thieno[3,2-b]pyridin-2-yl)prop-2- |
| | yn-1-yl)amino)-3-methoxy-N-methylbenzamide; |
| 761 | 4-((3-(7-((4-(dimethylamino)cyclohexyl)amino)-3-ethylthieno[3,2-b]pyridin-2-yl)prop-2-yn-1-yl)amino)-3- |
| | methoxy-N-methylbenzamide; |
| 762 | 4-((3-(3-(cyclopropylmethyl)-7-((4-(dimethylamino)cyclohexyl)amino)thieno[3,2-b]pyridin-2-yl)prop-2- |
| | yn-1-yl)amino)-3-methoxybenzenesulfonamide; |
| 763 | 4-((3-(7-((4-(dimethylamino)cyclohexyl)amino)-3-(2,2,2-trifluoroethyl)thieno[3,2-b]pyridin-2-yl)prop-2- |
| | yn-1-yl)amino)-3-methoxybenzenesulfonamide; |
| 764 | 4-((3-(7-((4-(dimethylamino)cyclohexyl)amino)-3-ethylthieno[3,2-b]pyridin-2-yl)prop-2-yn-1-yl)amino)-3- |
| | methoxybenzenesulfonamide; |
| 765 | 3-(cyclopropylmethyl)-N-(3,3-difluoro-1-methylpiperidin-4-yl)-2-(3-((2-methoxy-4- |
| | (methylsulfonyl)phenyl)amino)prop-1-yn-1-yl)thieno[3,2-b]pyridin-7-amine; |
| 766 | N-(3,3-difluoro-1-methylpiperidin-4-yl)-2-(3-((2-methoxy-4-(methylsulfonyl)phenyl)amino)prop-1-yn-1- |
| | yl)-3-(2,2,2-trifluoroethyl)thieno[3,2-b]pyridin-7-amine; |
| 767 | N-(3,3-difluoro-1-methylpiperidin-4-yl)-3-ethyl-2-(3-((2-methoxy-4-(methylsulfonyl)phenyl)amino)prop-1- |
| | yn-1-yl)thieno[3,2-b]pyridin-7-amine; |
| 768 | 4-((3-(3-(cyclopropylmethyl)-7-((3,3-difluoro-1-methylpiperidin-4-yl)amino)thieno[3,2-b]pyridin-2- |
| | yl)prop-2-yn-1-yl)amino)-3-methoxy-N-methylbenzamide; |
| 769 | 4-((3-(7-((3,3-difluoro-1-methylpiperidin-4-yl)amino)-3-(2,2,2-trifluoroethyl)thieno[3,2-b]pyridin-2- |
| | yl)prop-2-yn-1-yl)amino)-3-methoxy-N-methylbenzamide; |
| 770 | 4-((3-(7-((3,3-difluoro-1-methylpiperidin-4-yl)amino)-3-ethylthieno[3,2-b]pyridin-2-yl)prop-2-yn-1- |
| | yl)amino)-3-methoxy-N-methylbenzamide; |
| 771 | 4-((3-(3-(cyclopropylmethyl)-7-((3,3-difluoro-1-methylpiperidin-4-yl)amino)thieno[3,2-b]pyridin-2- |
| | yl)prop-2-yn-1-yl)amino)-3-methoxybenzenesulfonamide; |
| 772 | 4-((3-(7-((3,3-difluoro-1-methylpiperidin-4-yl)amino)-3-(2,2,2-trifluoroethyl)thieno[3,2-b]pyridin-2- |
| | yl)prop-2-yn-1-yl)amino)-3-methoxybenzenesulfonamide; |
| 773 | 4-((3-(7-((3,3-difluoro-1-methylpiperidin-4-yl)amino)-3-ethylthieno[3,2-b]pyridin-2-yl)prop-2-yn-1- |
| | yl)amino)-3-methoxybenzenesulfonamide; |
| 774 | 3-(cyclopropylmethyl)-2-(3-((2-methoxy-4-(methylsulfonyl)phenyl)amino)prop-1-yn-1-yl)-N-(1- |
| | methylpiperidin-4-yl)thieno[3,2-c]pyridin-7-amine; |
| 775 | 4-((3-(3-(cyclopropylmethyl)-7-((1-methylpiperidin-4-yl)amino)thieno[3,2-c]pyridin-2-yl)prop-2-yn-1- |
| | yl)amino)-3-methoxy-N-methylbenzamide; |
| 776 | 4-((3-(3-(cyclopropylmethyl)-7-((1-methylpiperidin-4-yl)amino)thieno[3,2-c]pyridin-2-yl)prop-2-yn-1- |
| | yl)amino)-3-methoxybenzenesulfonamide; |
| 777 | 2-(3-((2-methoxy-4-(methylsulfonyl)phenyl)amino)prop-1-yn-1-yl)-N-(1-methylpiperidin-4-yl)-3-(2,2,2- |
| | trifluoroethyl)thieno[3,2-c]pyridin-7-amine; |
| 778 | 3-methoxy-N-methyl-4-((3-(7-((1-methylpiperidin-4-yl)amino)-3-(2,2,2-trifluoroethyl)thieno[3,2-c]pyridin- |
| | 2-yl)prop-2-yn-1-yl)amino)benzamide; |
| 779 | 3-methoxy-4-((3-(7-((1-methylpiperidin-4-yl)amino)-3-(2,2,2-trifluoroethyl)thieno[3,2-c]pyridin-2-yl)prop- |
| | 2-yn-1-yl)amino)benzenesulfonamide; |
| 780 | 3-ethyl-2-(3-((2-methoxy-4-(methylsulfonyl)phenyl)amino)prop-1-yn-1-yl)-N-(1-methylpiperidin-4- |
| | yl)thieno[3,2-c]pyridin-7-amine; |
| 781 | 4-((3-(3-ethyl-7-((1-methylpiperidin-4-yl)amino)thieno[3,2-c]pyridin-2-yl)prop-2-yn-1-yl)amino)-3- |
| | methoxy-N-methylbenzamide; |
| 782 | 4-((3-(3-ethyl-7-((1-methylpiperidin-4-yl)amino)thieno[3,2-c]pyridin-2-yl)prop-2-yn-1-yl)amino)-3- |
| | methoxybenzenesulfonamide; |
| 783 | 1-(4-((3-(cyclopropylmethyl)-2-(3-((2-methoxy-4-(methylsulfonyl)phenyl)amino)prop-1-yn-1- |
| | yl)thieno[3,2-c]pyridin-7-yl)amino)-3-fluoropiperidin-1-yl)-3-methoxypropan-2-ol; |
| 784 | 4-((3-(3-(cyclopropylmethyl)-7-((3-fluoro-1-(2-hydroxy-3-methoxypropyl)piperidin-4-yl)amino)thieno[3,2- |
| | c]pyridin-2-yl)prop-2-yn-1-yl)amino)-3-methoxy-N-methylbenzamide; |
| 785 | 4-((3-(3-(cyclopropylmethyl)-7-((3-fluoro-1-(2-hydroxy-3-methoxypropyl)piperidin-4-yl)amino)thieno[3,2- |
| | c]pyridin-2-yl)prop-2-yn-1-yl)amino)-3-methoxybenzenesulfonamide; |
| 786 | 1-(3-fluoro-4-((2-(3-((2-methoxy-4-(methylsulfonyl)phenyl)amino)prop-1-yn-1-yl)-3-(2,2,2- |
| | trifluoroethyl)thieno[3,2-c]pyridin-7-yl)amino)piperidin-1-yl)-3-methoxypropan-2-o1; |
| 787 | 4-((3-(7-((3-fluoro-1-(2-hydroxy-3-methoxypropyl)piperidin-4-yl)amino)-3-(2,2,2-trifluoroethyl)thieno[3,2- |
| | c]pyridin-2-yl)prop-2-yn-1-yl)amino)-3-methoxy-N-methylbenzamide; |
| 788 | 4-((3-(7-((3-fluoro-1-(2-hydroxy-3-methoxypropyl)piperidin-4-yl)amino)-3-(2,2,2-trifluoroethyl)thieno[3,2- |
| | c]pyridin-2-yl)prop-2-yn-1-yl)amino)-3-methoxybenzenesulfonamide; |
| 789 | 1-(4-((3-ethyl-2-(3-((2-methoxy-4-(methylsulfonyl)phenyl)amino)prop-1-yn-1-yl)thieno[3,2-c]pyridin-7- |
| | yl)amino)-3-fluoropiperidin-1-yl)-3-methoxypropan-2-ol; |
| 790 | 4-((3-(3-ethyl-7-((3-fluoro-1-(2-hydroxy-3-methoxypropyl)piperidin-4-yl)amino)thieno[3,2-c]pyridin-2- |
| | yl)prop-2-yn-1-yl)amino)-3-methoxy-N-methylbenzamide; |
| 791 | 4-((3-(3-ethyl-7-((3-fluoro-1-(2-hydroxy-3-methoxypropyl)piperidin-4-yl)amino)thieno[3,2-c]pyridin-2- |
| | yl)prop-2-yn-1-yl)amino)-3-methoxybenzenesulfonamide; |
| 792 | 3-(cyclopropylmethyl)-N-(3-fluoro-1-methylpiperidin-4-yl)-2-(3-((2-methoxy-4- |
| | (methylsulfonyl)phenyl)amino)prop-1-yn-1-yl)thieno[3,2-c]pyridin-7-amine; |
| 793 | 4-((3-(3-(cyclopropylmethyl)-7-((3-fluoro-1-methylpiperidin-4-yl)amino)thieno[3,2-c]pyridin-2-yl)prop-2- |
| | yn-1-yl)amino)-3-methoxy-N-methylbenzamide; |
| 794 | 4-((3-(3-(cyclopropylmethyl)-7-((3-fluoro-1-methylpiperidin-4-yl)amino)thieno[3,2-c]pyridin-2-yl)prop-2- |
| | yn-1-yl)amino)-3-methoxybenzenesulfonamide; |
| 795 | N-(3-fluoro-1-methylpiperidin-4-yl)-2-(3-((2-methoxy-4-(methylsulfonyl)phenyl)amino)prop-1-yn-1-yl)-3- |
| | (2,2,2-trifluoroethyl)thieno[3,2-c]pyridin-7-amine; |
| 796 | 4-((3-(7-((3-fluoro-1-methylpiperidin-4-yl)amino)-3-(2,2,2-trifluoroethyl)thieno[3,2-c]pyridin-2-yl)prop-2- |
| | yn-1-yl)amino)-3-methoxy-N-methylbenzamide; |
| 797 | 4-((3-(7-((3-fluoro-1-methylpiperidin-4-yl)amino)-3-(2,2,2-trifluoroethyl)thieno[3,2-c]pyridin-2-yl)prop-2- |
| | yn-1-yl)amino)-3-methoxybenzenesulfonamide; |
| 798 | 3-ethyl-N-(3-fluoro-1-methylpiperidin-4-yl)-2-(3-((2-methoxy-4-(methylsulfonyl)phenyl)amino)prop-1-yn- |
| | 1-yl)thieno[3,2-c]pyridin-7-amine; |
| 799 | 4-((3-(3-ethyl-7-((3-fluoro-1-methylpiperidin-4-yl)amino)thieno[3,2-c]pyridin-2-yl)prop-2-yn-1-yl)amino)- |
| | 3-methoxy-N-methylbenzamide; |
| 800 | 4-((3-(3-ethyl-7-((3-fluoro-1-methylpiperidin-4-yl)amino)thieno[3,2-c]pyridin-2-yl)prop-2-yn-1-yl)amino)- |
| | 3-methoxybenzenesulfonamide; |
| 801 | N1-(3-(cyclopropylmethyl)-2-(3-((2-methoxy-4-(methylsulfonyl)phenyl)amino)prop-1-yn-1-yl)thieno[3,2- |
| | c]pyridin-7-yl)-N4,N4-dimethylcyclohexane-1,4-diamine; |
| 802 | 4-((3-(3-(cyclopropylmethyl)-7-((4-(dimethylamino)cyclohexyl)amino)thieno[3,2-c]pyridin-2-yl)prop-2-yn- |
| | 1-yl)amino)-3-methoxy-N-methylbenzamide; |
| 803 | 4-((3-(3-(cyclopropylmethyl)-7-((4-(dimethylamino)cyclohexyl)amino)thieno[3,2-c]pyridin-2-yl)prop-2-yn- |
| | 1-yl)amino)-3-methoxybenzenesulfonamide; |
| 804 | N1-(2-(3-((2-methoxy-4-(methylsulfonyl)phenyl)amino)prop-1-yn-1-yl)-3-(2,2,2-trifluoroethyl)thieno[3,2- |
| | c]pyridin-7-yl)-N4,N4-dimethylcyclohexane-1,4-diamine; |
| 805 | 4-((3-(7-((4-(dimethylamino)cyclohexyl)amino)-3-(2,2,2-trifluoroethyl)thieno[3,2-c]pyridin-2-yl)prop-2- |
| | yn-1-yl)amino)-3-methoxy-N-methylbenzamide; |
| 806 | 4-((3-(7-((4-(dimethylamino)cyclohexyl)amino)-3-(2,2,2-trifluoroethyl)thieno[3,2-c]pyridin-2-yl)prop-2- |
| | yn-1-yl)amino)-3-methoxybenzenesulfonamide; |
| 807 | N1-(3-ethyl-2-(3-((2-methoxy-4-(methylsulfonyl)phenyl)amino)prop-1-yn-1-yl)thieno[3,2-c]pyridin-7-yl)- |
| | N4,N4-dimethylcyclohexane-1,4-diamine; |
| 808 | 4-((3-(7-((4-(dimethylamino)cyclohexyl)amino)-3-ethylthieno[3,2-c]pyridin-2-yl)prop-2-yn-1-yl)amino)-3- |
| | methoxy-N-methylbenzamide; |
| 809 | 4-((3-(7-((4-(dimethylamino)cyclohexyl)amino)-3-ethylthieno[3,2-c]pyridin-2-yl)prop-2-yn-1-yl)amino)-3- |
| | methoxybenzenesulfonamide; |
| 810 | 3-(cyclopropylmethyl)-N-(3,3-difluoro-1-methylpiperidin-4-yl)-2-(3-((2-methoxy-4- |
| | (methylsulfonyl)phenyl)amino)prop-1-yn-1-yl)thieno[3,2-c]pyridin-7-amine; |
| 811 | 4-((3-(3-(cyclopropylmethyl)-7-((3,3-difluoro-1-methylpiperidin-4-yl)amino)thieno[3,2-c]pyridin-2- |
| | yl)prop-2-yn-1-yl)amino)-3-methoxy-N-methylbenzamide; |
| 812 | 4-((3-(3-(cyclopropylmethyl)-7-((3,3-difluoro-1-methylpiperidin-4-yl)amino)thieno[3,2-c]pyridin-2- |
| | yl)prop-2-yn-1-yl)amino)-3-methoxybenzenesulfonamide; |
| 813 | N-(3,3-difluoro-1-methylpiperidin-4-yl)-2-(3-((2-methoxy-4-(methylsulfonyl)phenyl)amino)prop-1-yn-1- |
| | yl)-3-(2,2,2-trifluoroethyl)thieno[3,2-c]pyridin-7-amine; |
| 814 | 4-((3-(7-((3,3-difluoro-1-methylpiperidin-4-yl)amino)-3-(2,2,2-trifluoroethyl)thieno[3,2-c]pyridin-2- |
| | yl)prop-2-yn-1-yl)amino)-3-methoxy-N-methylbenzamide; |
| 815 | 4-((3-(7-((3,3-difluoro-1-methylpiperidin-4-yl)amino)-3-(2,2,2-trifluoroethyl)thieno[3,2-c]pyridin-2- |
| | yl)prop-2-yn-1-yl)amino)-3-methoxybenzenesulfonamide; |
| 816 | N-(3,3-difluoro-1-methylpiperidin-4-yl)-3-ethyl-2-(3-((2-methoxy-4-(methylsulfonyl)phenyl)amino)prop-1- |
| | yn-1-yl)thieno[3,2-c]pyridin-7-amine; |
| 817 | 4-((3-(7-((3,3-difluoro-1-methylpiperidin-4-yl)amino)-3-ethylthieno[3,2-c]pyridin-2-yl)prop-2-yn-1- |
| | yl)amino)-3-methoxy-N-methylbenzamide; |
| 818 | 4-((3-(7-((3,3-difluoro-1-methylpiperidin-4-yl)amino)-3-ethylthieno[3,2-c]pyridin-2-yl)prop-2-yn-1- |
| | yl)amino)-3-methoxybenzenesulfonamide; |
| 819 | 3-(cyclopropylmethyl)-2-(3-((2-methoxy-4-(methylsulfonyl)phenyl)amino)prop-1-yn-1-yl)-N-(1- |
| | methylpiperidin-4-yl)thieno[2,3-c]pyridin-7-amine; |
| 820 | 4-((3-(3-(cyclopropylmethyl)-7-((1-methylpiperidin-4-yl)amino)thieno[2,3-c]pyridin-2-yl)prop-2-yn-1- |
| | yl)amino)-3-methoxy-N-methylbenzamide; |
| 821 | 4-((3-(3-(cyclopropylmethyl)-7-((1-methylpiperidin-4-yl)amino)thieno[2,3-c]pyridin-2-yl)prop-2-yn-1- |
| | yl)amino)-3-methoxybenzenesulfonamide; |
| 822 | 1-(4-((3-(cyclopropylmethyl)-2-(3-((2-methoxy-4-(methylsulfonyl)phenyl)amino)prop-1-yn-1- |
| | yl)thieno[2,3-c]pyridin-7-yl)amino)-3-fluoropiperidin-1-yl)-3-methoxypropan-2-o1; |
| 823 | 4-((3-(3-(cyclopropylmethyl)-7-((3-fluoro-1-(2-hydroxy-3-methoxypropyl)piperidin-4-yl)amino)thieno [2,3- |
| | c]pyridin-2-yl)prop-2-yn-1-yl)amino)-3-methoxy-N-methylbenzamide; |
| 824 | 4-((3-(3-(cyclopropylmethyl)-7-((3-fluoro-1-(2-hydroxy-3-methoxypropyl)piperidin-4-yl)amino)thieno[2,3- |
| | c]pyridin-2-yl)prop-2-yn-1-yl)amino)-3-methoxybenzenesulfonamide; |
| 825 | 3-(cyclopropylmethyl)-N-(3-fluoro-1-methylpiperidin-4-yl)-2-(3-((2-methoxy-4- |
| | (methylsulfonyl)phenyl)amino)prop-1-yn-1-yl)thieno[2,3-c]pyridin-7-amine; |
| 826 | 4-((3-(3-(cyclopropylmethyl)-7-((3-fluoro-1-methylpiperidin-4-yl)amino)thieno[2,3-c]pyridin-2-yl)prop-2- |
| | yn-1-yl)amino)-3-methoxy-N-methylbenzamide; |
| 827 | 4-((3-(3-(cyclopropylmethyl)-7-((3-fluoro-1-methylpiperidin-4-yl)amino)thieno[2,3-c]pyridin-2-yl)prop-2- |
| | yn-1-yl)amino)-3-methoxybenzenesulfonamide; |
| 828 | N1-(3-(cyclopropylmethyl)-2-(3-((2-methoxy-4-(methylsulfonyl)phenyl)amino)prop-1-yn-1-yl)thieno[2,3- |
| | c]pyridin-7-yl)-N4,N4-dimethylcyclohexane-1,4-diamine; |
| 829 | 4-((3-(3-(cyclopropylmethyl)-7-((4-(dimethylamino)cyclohexyl)amino)thieno[2,3-c]pyridin-2-yl)prop-2-yn- |
| | 1-yl)amino)-3-methoxy-N-methylbenzamide; |
| 830 | 4-((3-(3-(cyclopropylmethyl)-7-((4-(dimethylamino)cyclohexyl)amino)thieno[2,3-c]pyridin-2-yl)prop-2-yn- |
| | 1-yl)amino)-3-methoxybenzenesulfonamide; |
| 831 | 3-(cyclopropylmethyl)-N-(3,3-difluoro-1-methylpiperidin-4-yl)-2-(3-((2-methoxy-4- |
| | (methylsulfonyl)phenyl)amino)prop-1-yn-1-yl)thieno[2,3-c]pyridin-7-amine; |
| 832 | 4-((3-(3-(cyclopropylmethyl)-7-((3,3-difluoro-1-methylpiperidin-4-yl)amino)thieno[2,3-c]pyridin-2- |
| | yl)prop-2-yn-1-yl)amino)-3-methoxy-N-methylbenzamide; |
| 833 | 4-((3-(3-(cyclopropylmethyl)-7-((3,3-difluoro-1-methylpiperidin-4-yl)amino)thieno[2,3-c]pyridin-2- |
| | yl)prop-2-yn-1-yl)amino)-3-methoxybenzenesulfonamide; |
| 834 | 2-(3-((2-methoxy-4-(methylsulfonyl)phenyl)amino)prop-1-yn-1-yl)-N-(1-methylpiperidin-4-yl)-3-(2,2,2- |
| | trifluoroethyl)thieno[2,3-c]pyridin-7-amine; |
| 835 | 3-methoxy-N-methyl-4-((3-(7-((1-methylpiperidin-4-yl)amino)-3-(2,2,2-trifluoroethyl)thieno[2,3-c]pyridin- |
| | 2-yl)prop-2-yn-1-yl)amino)benzamide; |
| 836 | 3-methoxy-4-((3-(7-((1-methylpiperidin-4-yl)amino)-3-(2,2,2-trifluoroethyl)thieno[2,3-c]pyridin-2-yl)prop- |
| | 2-yn-1-yl)amino)benzenesulfonamide; |
| 837 | 1-(3-fluoro-4-((2-(3-((2-methoxy-4-(methylsulfonyl)phenyl)amino)prop-1-yn-1-yl)-3-(2,2,2- |
| | trifluoroethyl)thieno[2,3-c]pyridin-7-yl)amino)piperidin-1-yl)-3-methoxypropan-2-ol; |
| 838 | 4-((3-(7-((3-fluoro-1-(2-hydroxy-3-methoxypropyl)piperidin-4-yl)amino)-3-(2,2,2-trifluoroethyl)thieno[2,3- |
| | c]pyridin-2-yl)prop-2-yn-1-yl)amino)-3-methoxy-N-methylbenzamide; |
| 839 | 4-((3-(7-((3-fluoro-1-(2-hydroxy-3-methoxypropyl)piperidin-4-yl)amino)-3-(2,2,2-trifluoroethyl)thieno[2,3- |
| | c]pyridin-2-yl)prop-2-yn-1-yl)amino)-3-methoxybenzenesulfonamide; |
| 840 | N-(3-fluoro-1-methylpiperidin-4-yl)-2-(3-((2-methoxy-4-(methylsulfonyl)phenyl)amino)prop-1-yn-1-yl)-3- |
| | (2,2,2-trifluoroethyl)thieno[2,3-c]pyridin-7-amine; |
| 841 | 4-((3-(7-((3-fluoro-1-methylpiperidin-4-yl)amino)-3-(2,2,2-trifluoroethyl)thieno[2,3-c]pyridin-2-yl)prop-2- |
| | yn-1-yl)amino)-3-methoxy-N-methylbenzamide; |
| 842 | 4-((3-(7-((3-fluoro-1-methylpiperidin-4-yl)amino)-3-(2,2,2-trifluoroethyl)thieno[2,3-c]pyridin-2-yl)prop-2- |
| | yn-1-yl)amino)-3-methoxybenzenesulfonamide; |
| 843 | N1-(2-(3-((2-methoxy-4-(methylsulfonyl)phenyl)amino)prop-1-yn-1-yl)-3-(2,2,2-trifluoroethyl)thieno[2,3- |
| | c]pyridin-7-yl)-N4,N4-dimethylcyclohexane-1,4-diamine; |
| 844 | 4-((3-(7-((4-(dimethylamino)cyclohexyl)amino)-3-(2,2,2-trifluoroethyl)thieno[2,3-c]pyridin-2-yl)prop-2- |
| | yn-1-yl)amino)-3-methoxy-N-methylbenzamide; |
| 845 | 4-((3-(7-((4-(dimethylamino)cyclohexyl)amino)-3-(2,2,2-trifluoroethyl)thieno[2,3-c]pyridin-2-yl)prop-2- |
| | yn-1-yl)amino)-3-methoxybenzenesulfonamide; |
| 846 | N-(3,3-difluoro-1-methylpiperidin-4-yl)-2-(3-((2-methoxy-4-(methylsulfonyl)phenyl)amino)prop-1-yn-1- |
| | yl)-3-(2,2,2-trifluoroethyl)thieno[2,3-c]pyridin-7-amine; |
| 847 | 4-((3-(7-((3,3-difluoro-1-methylpiperidin-4-yl)amino)-3-(2,2,2-trifluoroethyl)thieno[2,3-c]pyridin-2- |
| | yl)prop-2-yn-1-yl)amino)-3-methoxy-N-methylbenzamide; |
| 848 | 4-((3-(7-((3,3-difluoro-1-methylpiperidin-4-yl)amino)-3-(2,2,2-trifluoroethyl)thieno[2,3-c]pyridin-2- |
| | yl)prop-2-yn-1-yl)amino)-3-methoxybenzenesulfonamide; |
| 849 | 3-ethyl-2-(3-((2-methoxy-4-(methylsulfonyl)phenyl)amino)prop-1-yn-1-yl)-N-(1-methylpiperidin-4- |
| | yl)thieno[2,3-c]pyridin-7-amine; |
| 850 | 4-((3-(3-ethyl-7-((1-methylpiperidin-4-yl)amino)thieno[2,3-c]pyridin-2-yl)prop-2-yn-1-yl)amino)-3- |
| | methoxy-N-methylbenzamide; |
| 851 | 4-((3-(3-ethyl-7-((1-methylpiperidin-4-yl)amino)thieno[2,3-c]pyridin-2-yl)prop-2-yn-1-yl)amino)-3- |
| | methoxybenzenesulfonamide; |
| 852 | 1-(4-((3-ethyl-2-(3-((2-methoxy-4-(methylsulfonyl)phenyl)amino)prop-1-yn-1-yl)thieno[2,3-c]pyridin-7- |
| | yl)amino)-3-fluoropiperidin-1-yl)-3-methoxypropan-2-ol; |
| 853 | 4-((3-(3-ethyl-7-((3-fluoro-1-(2-hydroxy-3-methoxypropyl)piperidin-4-yl)amino)thieno[2,3-c]pyridin-2- |
| | yl)prop-2-yn-1-yl)amino)-3-methoxy-N-methylbenzamide; |
| 854 | 4-((3-(3-ethyl-7-((3-fluoro-1-(2-hydroxy-3-methoxypropyl)piperidin-4-yl)amino)thieno[2,3-c]pyridin-2- |
| | yl)prop-2-yn-1-yl)amino)-3-methoxybenzenesulfonamide; |
| 855 | 3-ethyl-N-(3-fluoro-1-methylpiperidin-4-yl)-2-(3-((2-methoxy-4-(methylsulfonyl)phenyl)amino)prop-1-yn- |
| | 1-yl)thieno[2,3-c]pyridin-7-amine; |
| 856 | 4-((3-(3-ethyl-7-((3-fluoro-1-methylpiperidin-4-yl)amino)thieno[2,3-c]pyridin-2-yl)prop-2-yn-1-yl)amino)- |
| | 3-methoxy-N-methylbenzamide; |
| 857 | 4-((3-(3-ethyl-7-((3-fluoro-1-methylpiperidin-4-yl)amino)thieno[2,3-c]pyridin-2-yl)prop-2-yn-1-yl)amino)- |
| | 3-methoxybenzenesulfonamide; |
| 858 | N1-(3-ethyl-2-(3-((2-methoxy-4-(methylsulfonyl)phenyl)amino)prop-1-yn-1-yl)thieno[2,3-c]pyridin-7-yl)- |
| | N4,N4-dimethylcyclohexane-1,4-diamine; |
| 859 | 4-((3-(7-((4-(dimethylamino)cyclohexyl)amino)-3-ethylthieno[2,3-c]pyridin-2-yl)prop-2-yn-1-yl)amino)-3- |
| | methoxy-N-methylbenzamide; |
| 860 | 4-((3-(7-((4-(dimethylamino)cyclohexyl)amino)-3-ethylthieno[2,3-c]pyridin-2-yl)prop-2-yn-1-yl)amino)-3- |
| | methoxybenzenesulfonamide; |
| 861 | N-(3,3-difluoro-1-methylpiperidin-4-yl)-3-ethyl-2-(3-((2-methoxy-4-(methylsulfonyl)phenyl)amino)prop-1- |
| | yn-1-yl)thieno[2,3-c]pyridin-7-amine; |
| 862 | 4-((3-(7-((3,3-difluoro-1-methylpiperidin-4-yl)amino)-3-ethylthieno[2,3-c]pyridin-2-yl)prop-2-yn-1- |
| | yl)amino)-3-methoxy-N-methylbenzamide; |
| 863 | 4-((3-(7-((3,3-difluoro-1-methylpiperidin-4-yl)amino)-3-ethylthieno[2,3-c]pyridin-2-yl)prop-2-yn-1- |
| | yl)amino)-3-methoxybenzenesulfonamide; |
| 864 | 4-((3-(3-(cyanomethyl)-7-((3,3-difluoro-1-methylpiperidin-4-yl)amino)-1,1-dioxidobenzo[b]thiophen-2- |
| | yl)prop-2-yn-1-yl)amino)-3-methoxybenzenesulfonamide; |
| 865 | 4-((3-(3-(cyanomethyl)-7-((1,1-dioxidotetrahydro-2H-thiopyran-4-yl)amino)-1,1-dioxidobenzo[b]thiophen- |
| | 2-yl)prop-2-yn-1-yl)amino)-3-methoxybenzenesulfonamide; |
| 866 | 4-((3-(3-(cyanomethyl)-7-(((3R,4S)-3-fluoro-1-methylpiperidin-4-yl)amino)-1,1-dioxidobenzo[b]thiophen- |
| | 2-yl)prop-2-yn-1-yl)amino)-3-methoxybenzenesulfonamide; |
| 867 | 4-((3-(3-(cyanomethyl)-7-((3-fluoro-1-methylpiperidin-4-yl)amino)-1,1-dioxidobenzo[b]thiophen-2- |
| | yl)prop-2-yn-1-yl)amino)-3-methoxybenzenesulfonamide; |
| 868 | 4-((3-(3-(cyanomethyl)-7-((1-(dimethylglycyl)piperidin-4-yl)amino)-1,1-dioxidobenzo[b]thiophen-2- |
| | yl)prop-2-yn-1-yl)amino)-3-methoxybenzenesulfonamide; |
| 869 | 4-((3-(3-(cyanomethyl)-7-((3-fluoro-1-(2-hydroxy-3-methoxypropyl)piperidin-4-yl)amino)-1,1- |
| | dioxidobenzo[b]thiophen-2-yl)prop-2-yn-1-yl)amino)-3-methoxybenzenesulfonamide; |
| 870 | 4-((3-(3-(cyanomethyl)-7-(((3R,4R)-3-fluoro-1-methylpiperidin-4-yl)amino)-1,1-dioxidobenzo[b]thiophen- |
| | 2-yl)prop-2-yn-1-yl)amino)-3-methoxybenzenesulfonamide; |
| 871 | 4-((3-(3-(cyanomethyl)-7-(((3S,4S)-3-fluoro-1-methylpiperidin-4-yl)amino)-1,1-dioxidobenzo[b]thiophen- |
| | 2-yl)prop-2-yn-1-yl)amino)-3-methoxybenzenesulfonamide; |
| 872 | 4-((3-(3-(cyanomethyl)-7-((4-(dimethylamino)cyclohexyl)amino)-1,1-dioxidobenzo[b]thiophen-2-yl)prop- |
| | 2-yn-1-yl)amino)-3-methoxybenzenesulfonamide; |
| 873 | 4-((3-(3-(cyanomethyl)-7-((2-(dimethylamino)ethyl)amino)-1,1-dioxidobenzo[b]thiophen-2-yl)prop-2-yn-1- |
| | yl)amino)-3-methoxybenzenesulfonamide; |
| 874 | 4-((3-(3-(cyanomethyl)-7-(((3S,4R)-3-fluoro-1-methylpiperidin-4-yl)amino)-1,1-dioxidobenzo[b]thiophen- |
| | 2-yl)prop-2-yn-1-yl)amino)-3-methoxybenzenesulfonamide; |
| 875 | 4-((3-(3-(cyanomethyl)-7-((1-methylpiperidin-4-yl)amino)-1,1-dioxidobenzo[b]thiophen-2-yl)prop-2-yn-1- |
| | yl)amino)-3-methoxybenzenesulfonamide; |
| 876 | 4-((3-(3-(cyanomethyl)-1,1-dioxido-7-((tetrahydro-2H-pyran-4-yl)amino)benzo[b]thiophen-2-yl)prop-2-yn- |
| | 1-yl)amino)-3-methoxybenzenesulfonamide; |
| 877 | 4-((3-(7-((3,3-difluoro-1-methylpiperidin-4-yl)amino)-1,1-dioxido-3-(thiazol-4-yl)benzo[b]thiophen-2- |
| | yl)prop-2-yn-1-yl)amino)-3-methoxybenzenesulfonamide; |
| 878 | 4-((3-(7-((1,1-dioxidotetrahydro-2H-thiopyran-4-yl)amino)-1,1-dioxido-3-(thiazol-4-yl)benzo[b]thiophen- |
| | 2-yl)prop-2-yn-1-yl)amino)-3-methoxybenzenesulfonamide; |
| 879 | 4-((3-(7-(((3R,4S)-3-fluoro-1-methylpiperidin-4-yl)amino)-1,1-dioxido-3-(thiazol-4-yl)benzo[b]thiophen-2- |
| | yl)prop-2-yn-1-yl)amino)-3-methoxybenzenesulfonamide; |
| 880 | 4-((3-(7-((3-fluoro-1-methylpiperidin-4-yl)amino)-1,1-dioxido-3-(thiazol-4-yl)benzo[b]thiophen-2-yl)prop- |
| | 2-yn-1-yl)amino)-3-methoxybenzenesulfonamide; |
| 881 | 4-((3-(7-((1-(dimethylglycyl)piperidin-4-yl)amino)-1,1-dioxido-3-(thiazol-4-yl)benzo[b]thiophen-2- |
| | yl)prop-2-yn-1-yl)amino)-3-methoxybenzenesulfonamide; |
| 882 | 4-((3-(7-((3-fluoro-1-(2-hydroxy-3-methoxypropyl)piperidin-4-yl)amino)-1,1-dioxido-3-(thiazol-4- |
| | yl)benzo[b]thiophen-2-yl)prop-2-yn-1-yl)amino)-3-methoxybenzenesulfonamide; |
| 883 | 4-((3-(7-(((3R,4R)-3-fluoro-1-methylpiperidin-4-yl)amino)-1,1-dioxido-3-(thiazol-4-yl)benzo[b]thiophen-2- |
| | yl)prop-2-yn-1-yl)amino)-3-methoxybenzenesulfonamide; |
| 884 | 4-((3-(7-(((3S,4S)-3-fluoro-1-methylpiperidin-4-yl)amino)-1,1-dioxido-3-(thiazol-4-yl)benzo[b]thiophen-2- |
| | yl)prop-2-yn-1-yl)amino)-3-methoxybenzenesulfonamide; |
| 885 | 4-((3-(7-((4-(dimethylamino)cyclohexyl)amino)-1,1-dioxido-3-(thiazol-4-yl)benzo[b]thiophen-2-yl)prop-2- |
| | yn-1-yl)amino)-3-methoxybenzenesulfonamide; |
| 886 | 4-((3-(7-((2-(dimethylamino)ethyl)amino)-1,1-dioxido-3-(thiazol-4-yl)benzo[b]thiophen-2-yl)prop-2-yn-1- |
| | yl)amino)-3-methoxybenzenesulfonamide; |
| 887 | 4-((3-(7-(((3S,4R)-3-fluoro-1-methylpiperidin-4-yl)amino)-1,1-dioxido-3-(thiazol-4-yl)benzo[b]thiophen-2- |
| | yl)prop-2-yn-1-yl)amino)-3-methoxybenzenesulfonamide; |
| 888 | 3-methoxy-4-((3-(7-((1-methylpiperidin-4-yl)amino)-1,1-dioxido-3-(thiazol-4-yl)benzo[b]thiophen-2- |
| | yl)prop-2-yn-1-yl)amino)benzenesulfonamide; |
| 889 | 4-((3-(1,1-dioxido-7-((tetrahydro-2H-pyran-4-yl)amino)-3-(thiazol-4-yl)benzo[b]thiophen-2-yl)prop-2-yn- |
| | 1-yl)amino)-3-methoxybenzenesulfonamide; |
| 890 | 4-((3-(7-((3,3-difluoro-1-methylpiperidin-4-yl)amino)-1,1-dioxido-3-(2,2,2- |
| | trifluoroethyl)benzo[b]thiophen-2-yl)prop-2-yn-1-yl)amino)-3-methoxybenzenesulfonamide; |
| 891 | 4-((3-(7-((1,1-dioxidotetrahydro-2H-thiopyran-4-yl)amino)-1,1-dioxido-3-(2,2,2- |
| | trifluoroethyl)benzo[b]thiophen-2-yl)prop-2-yn-1-yl)amino)-3-methoxybenzenesulfonamide; |
| 892 | 4-((3-(7-(((3R,4S)-3-fluoro-1-methylpiperidin-4-yl)amino)-1,1-dioxido-3-(2,2,2- |
| | trifluoroethyl)benzo[b]thiophen-2-yl)prop-2-yn-1-yl)amino)-3-methoxybenzenesulfonamide; |
| 893 | 4-((3-(7-((3-fluoro-1-methylpiperidin-4-yl)amino)-1,1-dioxido-3-(2,2,2-trifluoroethyl)benzo[b]thiophen-2- |
| | yl)prop-2-yn-1-yl)amino)-3-methoxybenzenesulfonamide; |
| 894 | 4-((3-(7-((1-(dimethylglycyl)piperidin-4-yl)amino)-1,1-dioxido-3-(2,2,2-trifluoroethyl)benzo[b]thiophen-2- |
| | yl)prop-2-yn-1-yl)amino)-3-methoxybenzenesulfonamide; |
| 895 | 4-((3-(7-((3-fluoro-1-(2-hydroxy-3-methoxypropyl)piperidin-4-yl)amino)-1,1-dioxido-3-(2,2,2- |
| | trifluoroethyl)benzo[b]thiophen-2-yl)prop-2-yn-1-yl)amino)-3-methoxybenzenesulfonamide; |
| 896 | 4-((3-(7-(((3R,4R)-3-fluoro-1-methylpiperidin-4-yl)amino)-1,1-dioxido-3-(2,2,2- |
| | trifluoroethyl)benzo[b]thiophen-2-yl)prop-2-yn-1-yl)amino)-3-methoxybenzenesulfonamide; |
| 897 | 4-((3-(7-(((3S,4S)-3-fluoro-1-methylpiperidin-4-yl)amino)-1,1-dioxido-3-(2,2,2- |
| | trifluoroethyl)benzo[b]thiophen-2-yl)prop-2-yn-1-yl)amino)-3-methoxybenzenesulfonamide; |
| 898 | 4-((3-(7-((4-(dimethylamino)cyclohexyl)amino)-1,1-dioxido-3-(2,2,2-trifluoroethyl)benzo[b]thiophen-2- |
| | yl)prop-2-yn-1-yl)amino)-3-methoxybenzenesulfonamide; |
| 899 | 4-((3-(7-((2-(dimethylamino)ethyl)amino)-1,1-dioxido-3-(2,2,2-trifluoroethyl)benzo[b]thiophen-2-yl)prop- |
| | 2-yn-1-yl)amino)-3-methoxybenzenesulfonamide; |
| 900 | 4-((3-(7-(((3S,4R)-3-fluoro-1-methylpiperidin-4-yl)amino)-1,1-dioxido-3-(2,2,2- |
| | trifluoroethyl)benzo[b]thiophen-2-yl)prop-2-yn-1-yl)amino)-3-methoxybenzenesulfonamide; |
| 901 | 3-methoxy-4-((3-(7-((1-methylpiperidin-4-yl)amino)-1,1-dioxido-3-(2,2,2-trifluoroethyl)benzo[b]thiophen- |
| | 2-yl)prop-2-yn-1-yl)amino)benzenesulfonamide; |
| 902 | 4-((3-(1,1-dioxido-7-((tetrahydro-2H-pyran-4-yl)amino)-3-(2,2,2-trifluoroethyl)benzo[b]thiophen-2- |
| | yl)prop-2-yn-1-yl)amino)-3-methoxybenzenesulfonamide; |
| 903 | 4-((3-(3-(cyclopropylmethyl)-7-((3,3-difluoro-1-methylpiperidin-4-yl)amino)-1,1-dioxidobenzo[b]thiophen- |
| | 2-yl)prop-2-yn-1-yl)amino)-3-methoxybenzenesulfonamide; |
| 904 | 4-((3-(3-(cyclopropylmethyl)-7-((1,1-dioxidotetrahydro-2H-thiopyran-4-yl)amino)-1,1- |
| | dioxidobenzo[b]thiophen-2-yl)prop-2-yn-1-yl)amino)-3-methoxybenzenesulfonamide; |
| 905 | 4-((3-(3-(cyclopropylmethyl)-7-(((3R,4S)-3-fluoro-1-methylpiperidin-4-yl)amino)-1,1- |
| | dioxidobenzo[b]thiophen-2-yl)prop-2-yn-1-yl)amino)-3-methoxybenzenesulfonamide; |
| 906 | 4-((3-(3-(cyclopropylmethyl)-7-((3-fluoro-1-methylpiperidin-4-yl)amino)-1,1-dioxidobenzo[b]thiophen-2- |
| | yl)prop-2-yn-1-yl)amino)-3-methoxybenzenesulfonamide; |
| 907 | 4-((3-(3-(cyclopropylmethyl)-7-((1-(dimethylglycyl)piperidin-4-yl)amino)-1,1-dioxidobenzo[b]thiophen-2- |
| | yl)prop-2-yn-1-yl)amino)-3-methoxybenzenesulfonamide; |
| 908 | 4-((3-(3-(cyclopropylmethyl)-7-((3-fluoro-1-(2-hydroxy-3-methoxypropyl)piperidin-4-yl)amino)-1,1- |
| | dioxidobenzo[b]thiophen-2-yl)prop-2-yn-1-yl)amino)-3-methoxybenzenesulfonamide; |
| 909 | 4-((3-(3-(cyclopropylmethyl)-7-(((3R,4R)-3-fluoro-1-methylpiperidin-4-yl)amino)-1,1- |
| | dioxidobenzo[b]thiophen-2-yl)prop-2-yn-1-yl)amino)-3-methoxybenzenesulfonamide; |
| 910 | 4-((3-(3-(cyclopropylmethyl)-7-(((3S,4S)-3-fluoro-1-methylpiperidin-4-yl)amino)-1,1- |
| | dioxidobenzo[b]thiophen-2-yl)prop-2-yn-1-yl)amino)-3-methoxybenzenesulfonamide; |
| 911 | 4-((3-(3-(cyclopropylmethyl)-7-((4-(dimethylamino)cyclohexyl)amino)-1,1-dioxidobenzo[b]thiophen-2- |
| | yl)prop-2-yn-1-yl)amino)-3-methoxybenzenesulfonamide; |
| 912 | 4-((3-(3-(cyclopropylmethyl)-7-((2-(dimethylamino)ethyl)amino)-1,1-dioxidobenzo[b]thiophen-2-yl)prop- |
| | 2-yn-1-yl)amino)-3-methoxybenzenesulfonamide; |
| 913 | 4-((3-(3-(cyclopropylmethyl)-7-(((3S,4R)-3-fluoro-1-methylpiperidin-4-yl)amino)-1,1- |
| | dioxidobenzo[b]thiophen-2-yl)prop-2-yn-1-yl)amino)-3-methoxybenzenesulfonamide; |
| 914 | 4-((3-(3-(cyclopropylmethyl)-7-((1-methylpiperidin-4-yl)amino)-1,1-dioxidobenzo[b]thiophen-2-yl)prop-2- |
| | yn-1-yl)amino)-3-methoxybenzenesulfonamide; |
| 915 | 4-((3-(3-(cyclopropylmethyl)-1,1-dioxido-7-((tetrahydro-2H-pyran-4-yl)amino)benzo[b]thiophen-2- |
| | yl)prop-2-yn-1-yl)amino)-3-methoxybenzenesulfonamide; |
| 916 | 4-((3-(7-((3,3-difluoro-1-methylpiperidin-4-yl)amino)-3-(2,2-difluoroethyl)-1,1-dioxidobenzo[b]thiophen- |
| | 2-yl)prop-2-yn-1-yl)amino)-3-methoxybenzenesulfonamide; |
| 917 | 4-((3-(3-(2,2-difluoroethyl)-7-((1,1-dioxidotetrahydro-2H-thiopyran-4-yl)amino)-1,1- |
| | dioxidobenzo[b]thiophen-2-yl)prop-2-yn-1-yl)amino)-3-methoxybenzenesulfonamide; |
| 918 | 4-((3-(3-(2,2-difluoroethyl)-7-(((3R,4S)-3-fluoro-1-methylpiperidin-4-yl)amino)-1,1- |
| | dioxidobenzo[b]thiophen-2-yl)prop-2-yn-1-yl)amino)-3-methoxybenzenesulfonamide; |
| 919 | 4-((3-(3-(2,2-difluoroethyl)-7-((3-fluoro-1-methylpiperidin-4-yl)amino)-1,1-dioxidobenzo[b]thiophen-2- |
| | yl)prop-2-yn-1-yl)amino)-3-methoxybenzenesulfonamide; |
| 920 | 4-((3-(3-(2,2-difluoroethyl)-7-((1-(dimethylglycyl)piperidin-4-yl)amino)-1,1-dioxidobenzo[b]thiophen-2- |
| | yl)prop-2-yn-1-yl)amino)-3-methoxybenzenesulfonamide; |
| 921 | 4-((3-(3-(2,2-difluoroethyl)-7-((3-fluoro-1-(2-hydroxy-3-methoxypropyl)piperidin-4-yl)amino)-1,1- |
| | dioxidobenzo[b]thiophen-2-yl)prop-2-yn-1-yl)amino)-3-methoxybenzenesulfonamide; |
| 922 | 4-((3-(3-(2,2-difluoroethyl)-7-(((3R,4R)-3-fluoro-1-methylpiperidin-4-yl)amino)-1,1- |
| | dioxidobenzo[b]thiophen-2-yl)prop-2-yn-1-yl)amino)-3-methoxybenzenesulfonamide; |
| 923 | 4-((3-(3-(2,2-difluoroethyl)-7-(((3S,4S)-3-fluoro-1-methylpiperidin-4-yl)amino)-1,1- |
| | dioxidobenzo[b]thiophen-2-yl)prop-2-yn-1-yl)amino)-3-methoxybenzenesulfonamide; |
| 924 | 4-((3-(3-(2,2-difluoroethyl)-7-((4-(dimethylamino)cyclohexyl)amino)-1,1-dioxidobenzo[b]thiophen-2- |
| | yl)prop-2-yn-1-yl)amino)-3-methoxybenzenesulfonamide; |
| 925 | 4-((3-(3-(2,2-difluoroethyl)-7-((2-(dimethylamino)ethyl)amino)-1,1-dioxidobenzo[b]thiophen-2-yl)prop-2- |
| | yn-1-yl)amino)-3-methoxybenzenesulfonamide; |
| 926 | 4-((3-(3-(2,2-difluoroethyl)-7-(((3S,4R)-3-fluoro-1-methylpiperidin-4-yl)amino)-1,1- |
| | dioxidobenzo[b]thiophen-2-yl)prop-2-yn-1-yl)amino)-3-methoxybenzenesulfonamide; |
| 927 | 4-((3-(3-(2,2-difluoroethyl)-7-((1-methylpiperidin-4-yl)amino)-1,1-dioxidobenzo[b]thiophen-2-yl)prop-2- |
| | yn-1-yl)amino)-3-methoxybenzenesulfonamide; |
| 928 | 4-((3-(3-(2,2-difluoroethyl)-1,1-dioxido-7-((tetrahydro-2H-pyran-4-yl)amino)benzo[b]thiophen-2-yl)prop- |
| | 2-yn-1-yl)amino)-3-methoxybenzenesulfonamide; |
| 929 | 4-((3-(7-((3,3-difluoro-1-methylpiperidin-4-yl)amino)-1,1-dioxido-3-(1H-pyrrol-1-yl)benzo[b]thiophen-2- |
| | yl)prop-2-yn-1-yl)amino)-3-methoxybenzenesulfonamide; |
| 930 | 4-((3-(7-((1,1-dioxidotetrahydro-2H-thiopyran-4-yl)amino)-1,1-dioxido-3-(1H-pyrrol-1- |
| | yl)benzo[b]thiophen-2-yl)prop-2-yn-1-yl)amino)-3-methoxybenzenesulfonamide; |
| 931 | 4-((3-(7-(((3R,4S)-3-fluoro-1-methylpiperidin-4-yl)amino)-1,1-dioxido-3-(1H-pyrrol-1- |
| | yl)benzo[b]thiophen-2-yl)prop-2-yn-1-yl)amino)-3-methoxybenzenesulfonamide; |
| 932 | 4-((3-(7-((3-fluoro-1-methylpiperidin-4-yl)amino)-1,1-dioxido-3-(1H-pyrrol-1-yl)benzo[b]thiophen-2- |
| | yl)prop-2-yn-1-yl)amino)-3-methoxybenzenesulfonamide; |
| 933 | 4-((3-(7-((1-(dimethylglycyl)piperidin-4-yl)amino)-1,1-dioxido-3-(1H-pyrrol-1-yl)benzo[b]thiophen-2- |
| | yl)prop-2-yn-1-yl)amino)-3-methoxybenzenesulfonamide; |
| 934 | 4-((3-(7-((3-fluoro-1-(2-hydroxy-3-methoxypropyl)piperidin-4-yl)amino)-1,1-dioxido-3-(1H-pyrrol-1- |
| | yl)benzo[b]thiophen-2-yl)prop-2-yn-1-yl)amino)-3-methoxybenzenesulfonamide; |
| 935 | 4-((3-(7-(((3R,4R)-3-fluoro-1-methylpiperidin-4-yl)amino)-1,1-dioxido-3-(1H-pyrrol-1- |
| | yl)benzo[b]thiophen-2-yl)prop-2-yn-1-yl)amino)-3-methoxybenzenesulfonamide; |
| 936 | 4-((3-(7-(((3S,4S)-3-fluoro-1-methylpiperidin-4-yl)amino)-1,1-dioxido-3-(1H-pyrrol-1- |
| | yl)benzo[b]thiophen-2-yl)prop-2-yn-1-yl)amino)-3-methoxybenzenesulfonamide; |
| 937 | 4-((3-(7-((4-(dimethylamino)cyclohexyl)amino)-1,1-dioxido-3-(1H-pyrrol-1-yl)benzo[b]thiophen-2- |
| | yl)prop-2-yn-1-yl)amino)-3-methoxybenzenesulfonamide; |
| 938 | 4-((3-(7-((2-(dimethylamino)ethyl)amino)-1,1-dioxido-3-(1H-pyrrol-1-yl)benzo[b]thiophen-2-yl)prop-2-yn- |
| | 1-yl)amino)-3-methoxybenzenesulfonamide; |
| 939 | 4-((3-(7-(((3S,4R)-3-fluoro-1-methylpiperidin-4-yl)amino)-1,1-dioxido-3-(1H-pyrrol-1- |
| | yl)benzo[b]thiophen-2-yl)prop-2-yn-1-yl)amino)-3-methoxybenzenesulfonamide; |
| 940 | 3-methoxy-4-((3-(7-((1-methylpiperidin-4-yl)amino)-1,1-dioxido-3-(1H-pyrrol-1-yl)benzo[b]thiophen-2- |
| | yl)prop-2-yn-1-yl)amino)benzenesulfonamide; |
| 941 | 4-((3-(1,1-dioxido-3-(1H-pyrrol-1-yl)-7-((tetrahydro-2H-pyran-4-yl)amino)benzo[b]thiophen-2-yl)prop-2- |
| | yn-1-yl)amino)-3-methoxybenzenesulfonamide; |
| 942 | 4-((3-(7-((3,3-difluoro-1-methylpiperidin-4-yl)amino)-3-ethyl-1,1-dioxidobenzo[b]thiophen-2-yl)prop-2- |
| | yn-1-yl)amino)-3-methoxybenzenesulfonamide; |
| 943 | 4-((3-(7-((1,1-dioxidotetrahydro-2H-thiopyran-4-yl)amino)-3-ethyl-1,1-dioxidobenzo[b]thiophen-2-yl)prop- |
| | 2-yn-1-yl)amino)-3-methoxybenzenesulfonamide; |
| 944 | 4-((3-(3-ethyl-7-(((3R,4S)-3-fluoro-1-methylpiperidin-4-yl)amino)-1,1-dioxidobenzo[b]thiophen-2-yl)prop- |
| | 2-yn-1-yl)amino)-3-methoxybenzenesulfonamide; |
| 945 | 4-((3-(3-ethyl-7-((3-fluoro-1-methylpiperidin-4-yl)amino)-1,1-dioxidobenzo[b]thiophen-2-yl)prop-2-yn-1- |
| | yl)amino)-3-methoxybenzenesulfonamide; |
| 946 | 4-((3-(7-((1-(dimethylglycyl)piperidin-4-yl)amino)-3-ethyl-1,1-dioxidobenzo[b]thiophen-2-yl)prop-2-yn-1- |
| | yl)amino)-3-methoxybenzenesulfonamide; |
| 947 | 4-((3-(3-ethyl-7-((3-fluoro-1-(2-hydroxy-3-methoxypropyl)piperidin-4-yl)amino)-1,1- |
| | dioxidobenzo[b]thiophen-2-yl)prop-2-yn-1-yl)amino)-3-methoxybenzenesulfonamide; |
| 948 | 4-((3-(3-ethyl-7-(((3R,4R)-3-fluoro-1-methylpiperidin-4-yl)amino)-1,1-dioxidobenzo[b]thiophen-2-yl)prop- |
| | 2-yn-1-yl)amino)-3-methoxybenzenesulfonamide; |
| 949 | 4-((3-(3-ethyl-7-(((3S,4S)-3-fluoro-1-methylpiperidin-4-yl)amino)-1,1-dioxidobenzo[b]thiophen-2-yl)prop- |
| | 2-yn-1-yl)amino)-3-methoxybenzenesulfonamide; |
| 950 | 4-((3-(7-((4-(dimethylamino)cyclohexyl)amino)-3-ethyl-1,1-dioxidobenzo[b]thiophen-2-yl)prop-2-yn-1- |
| | yl)amino)-3-methoxybenzenesulfonamide; |
| 951 | 4-((3-(7-((2-(dimethylamino)ethyl)amino)-3-ethyl-1,1-dioxidobenzo[b]thiophen-2-yl)prop-2-yn-1- |
| | yl)amino)-3-methoxybenzenesulfonamide; |
| 952 | 4-((3-(3-ethyl-7-(((3S,4R)-3-fluoro-1-methylpiperidin-4-yl)amino)-1,1-dioxidobenzo[b]thiophen-2-yl)prop- |
| | 2-yn-1-yl)amino)-3-methoxybenzenesulfonamide; |
| 953 | 4-((3-(3-ethyl-7-((1-methylpiperidin-4-yl)amino)-1,1-dioxidobenzo[b]thiophen-2-yl)prop-2-yn-1- |
| | yl)amino)-3-methoxybenzenesulfonamide; |
| 954 | 4-((3-(3-ethyl-1,1-dioxido-7-((tetrahydro-2H-pyran-4-yl)amino)benzo[b]thiophen-2-yl)prop-2-yn-1- |
| | yl)amino)-3-methoxybenzenesulfonamide; |
| 955 | 2-(7-((3,3-difluoro-1-methylpiperidin-4-yl)amino)-2-(3-((5-fluoro-2-methoxy-4- |
| | (methylsulfonyl)phenyl)amino)prop-1-yn-1-yl)-1,1-dioxidobenzo[b]thiophen-3-yl)acetonitrile; |
| 956 | 2-(7-((1,1-dioxidotetrahydro-2H-thiopyran-4-yl)amino)-2-(3-((5-fluoro-2-methoxy-4- |
| | (methylsulfonyl)phenyl)amino)prop-1-yn-1-yl)-1,1-dioxidobenzo[b]thiophen-3-yl)acetonitrile; |
| 957 | 2-(7-(((3R,4S)-3-fluoro-1-methylpiperidin-4-yl)amino)-2-(3-((5-fluoro-2-methoxy-4- |
| | (methylsulfonyl)phenyl)amino)prop-1-yn-1-yl)-1,1-dioxidobenzo[b]thiophen-3-yl)acetonitrile; |
| 958 | 2-(7-((3-fluoro-1-methylpiperidin-4-yl)amino)-2-(3-((5-fluoro-2-methoxy-4- |
| | (methylsulfonyl)phenyl)amino)prop-1-yn-1-yl)-1,1-dioxidobenzo[b]thiophen-3-yl)acetonitrile; |
| 959 | 2-(7-((1-(dimethylglycyl)piperidin-4-yl)amino)-2-(3-((5-fluoro-2-methoxy-4- |
| | (methylsulfonyl)phenyl)amino)prop-1-yn-1-yl)-1,1-dioxidobenzo[b]thiophen-3-yl)acetonitrile; |
| 960 | 2-(7-((3-fluoro-1-(2-hydroxy-3-methoxypropyl)piperidin-4-yl)amino)-2-(3-((5-fluoro-2-methoxy-4- |
| | (methylsulfonyl)phenyl)amino)prop-1-yn-1-yl)-1,1-dioxidobenzo[b]thiophen-3-yl)acetonitrile; |
| 961 | 2-(7-(((3R,4R)-3-fluoro-1-methylpiperidin-4-yl)amino)-2-(3-((5-fluoro-2-methoxy-4- |
| | (methylsulfonyl)phenyl)amino)prop-1-yn-1-yl)-1,1-dioxidobenzo[b]thiophen-3-yl)acetonitrile; |
| 962 | 2-(7-(((3S,4S)-3-fluoro-1-methylpiperidin-4-yl)amino)-2-(3-((5-fluoro-2-methoxy-4- |
| | (methylsulfonyl)phenyl)amino)prop-1-yn-1-yl)-1,1-dioxidobenzo[b]thiophen-3-yl)acetonitrile; |
| 963 | 2-(7-((4-(dimethylamino)cyclohexyl)amino)-2-(3-((5-fluoro-2-methoxy-4- |
| | (methylsulfonyl)phenyl)amino)prop-1-yn-1-yl)-1,1-dioxidobenzo[b]thiophen-3-yl)acetonitrile; |
| 964 | 2-(7-((2-(dimethylamino)ethyl)amino)-2-(3-((5-fluoro-2-methoxy-4-(methylsulfonyl)phenyl)amino)prop-1- |
| | yn-1-yl)-1,1-dioxidobenzo[b]thiophen-3-yl)acetonitrile; |
| 965 | 2-(7-(((3S,4R)-3-fluoro-1-methylpiperidin-4-yl)amino)-2-(3-((5-fluoro-2-methoxy-4- |
| | (methylsulfonyl)phenyl)amino)prop-1-yn-1-yl)-1,1-dioxidobenzo[b]thiophen-3-yl)acetonitrile; |
| 966 | 2-(2-(3-((5-fluoro-2-methoxy-4-(methylsulfonyl)phenyl)amino)prop-1-yn-1-yl)-7-((1-methylpiperidin-4- |
| | yl)amino)-1,1-dioxidobenzo[b]thiophen-3-yl)acetonitrile; |
| 967 | 2-(2-(3-((5-fluoro-2-methoxy-4-(methylsulfonyl)phenyl)amino)prop-1-yn-1-yl)-1,1-dioxido-7-((tetrahydro- |
| | 2H-pyran-4-yl)amino)benzo[b]thiophen-3-yl)acetonitrile; |
| 968 | 7-((3,3-difluoro-1-methylpiperidin-4-yl)amino)-2-(3-((5-fluoro-2-methoxy-4- |
| | (methylsulfonyl)phenyl)amino)prop-1-yn-1-yl)-3-(thiazol-4-yl)benzo[b]thiophene 1,1-dioxide; |
| 969 | 7-((1,1-dioxidotetrahydro-2H-thiopyran-4-yl)amino)-2-(3-((5-fluoro-2-methoxy-4- |
| | (methylsulfonyl)phenyl)amino)prop-1-yn-1-yl)-3-(thiazol-4-yl)benzo[b]thiophene 1,1-dioxide; |
| 970 | 7-(((3R,4S)-3-fluoro-1-methylpiperidin-4-yl)amino)-2-(3-((5-fluoro-2-methoxy-4- |
| | (methylsulfonyl)phenyl)amino)prop-1-yn-1-yl)-3-(thiazol-4-yl)benzo[b]thiophene 1,1-dioxide; |
| 971 | 7-((3-fluoro-1-methylpiperidin-4-yl)amino)-2-(3-((5-fluoro-2-methoxy-4- |
| | (methylsulfonyl)phenyl)amino)prop-1-yn-1-yl)-3-(thiazol-4-yl)benzo[b]thiophene 1,1-dioxide; |
| 972 | 2-(dimethylamino)-1-(4-((2-(3-((5-fluoro-2-methoxy-4-(methylsulfonyl)phenyl)amino)prop-1-yn-1-yl)-1,1- |
| | dioxido-3-(thiazol-4-yl)benzo[b]thiophen-7-yl)amino)piperidin-1-yl)ethan-1-one; |
| 973 | 7-((3-fluoro-1-(2-hydroxy-3-methoxypropyl)piperidin-4-yl)amino)-2-(3-((5-fluoro-2-methoxy-4- |
| | (methylsulfonyl)phenyl)amino)prop-1-yn-1-yl)-3-(thiazol-4-yl)benzo[b]thiophene 1,1-dioxide; |
| 974 | 7-(((3R,4R)-3-fluoro-1-methylpiperidin-4-yl)amino)-2-(3-((5-fluoro-2-methoxy-4- |
| | (methylsulfonyl)phenyl)amino)prop-1-yn-1-yl)-3-(thiazol-4-yl)benzo[b]thiophene 1,1-dioxide; |
| 975 | 7-(((3S,4S)-3-fluoro-1-methylpiperidin-4-yl)amino)-2-(3-((5-fluoro-2-methoxy-4- |
| | (methylsulfonyl)phenyl)amino)prop-1-yn-1-yl)-3-(thiazol-4-yl)benzo[b]thiophene 1,1-dioxide; |
| 976 | 7-((4-(dimethylamino)cyclohexyl)amino)-2-(3-((5-fluoro-2-methoxy-4- |
| | (methylsulfonyl)phenyl)amino)prop-1-yn-1-yl)-3-(thiazol-4-yl)benzo[b]thiophene 1,1-dioxide; |
| 977 | 7-((2-(dimethylamino)ethyl)amino)-2-(3-((5-fluoro-2-methoxy-4-(methylsulfonyl)phenyl)amino)prop-1-yn- |
| | 1-yl)-3-(thiazol-4-yl)benzo[b]thiophene 1,1-dioxide; |
| 978 | 7-(((3S,4R)-3-fluoro-1-methylpiperidin-4-yl)amino)-2-(3-((5-fluoro-2-methoxy-4- |
| | (methylsulfonyl)phenyl)amino)prop-1-yn-1-yl)-3-(thiazol-4-yl)benzo[b]thiophene 1,1-dioxide; |
| 979 | 2-(3-((5-fluoro-2-methoxy-4-(methylsulfonyl)phenyl)amino)prop-1-yn-1-yl)-7-((1-methylpiperidin-4- |
| | yl)amino)-3-(thiazol-4-yl)benzo[b]thiophene 1,1-dioxide; |
| 980 | 2-(3-((5-fluoro-2-methoxy-4-(methylsulfonyl)phenyl)amino)prop-1-yn-1-yl)-7-((tetrahydro-2H-pyran-4- |
| | yl)amino)-3-(thiazol-4-yl)benzo[b]thiophene 1,1-dioxide; |
| 981 | 7-((3,3-difluoro-1-methylpiperidin-4-yl)amino)-2-(3-((5-fluoro-2-methoxy-4- |
| | (methylsulfonyl)phenyl)amino)prop-1-yn-1-yl)-3-(2,2,2-trifluoroethyl)benzo[b]thiophene 1,1-dioxide; |
| 982 | 7-((1,1-dioxidotetrahydro-2H-thiopyran-4-yl)amino)-2-(3-((5-fluoro-2-methoxy-4- |
| | (methylsulfonyl)phenyl)amino)prop-1-yn-1-yl)-3-(2,2,2-trifluoroethyl)benzo[b]thiophene 1,1-dioxide; |
| 983 | 7-(((3R,4S)-3-fluoro-1-methylpiperidin-4-yl)amino)-2-(3-((5-fluoro-2-methoxy-4- |
| | (methylsulfonyl)phenyl)amino)prop-1-yn-1-yl)-3-(2,2,2-trifluoroethyl)benzo[b]thiophene 1,1-dioxide; |
| 984 | 7-((3-fluoro-1-methylpiperidin-4-yl)amino)-2-(3-((5-fluoro-2-methoxy-4- |
| | (methylsulfonyl)phenyl)amino)prop-1-yn-1-yl)-3-(2,2,2-trifluoroethyl)benzo[b]thiophene 1,1-dioxide; |
| 985 | 2-(dimethylamino)-1-(4-((2-(3-((5-fluoro-2-methoxy-4-(methylsulfonyl)phenyl)amino)prop-1-yn-1-yl)-1,1- |
| | dioxido-3-(2,2,2-trifluoroethyl)benzo[b]thiophen-7-yl)amino)piperidin-1-yl)ethan-1-one; |
| 986 | 7-((3-fluoro-1-(2-hydroxy-3-methoxypropyl)piperidin-4-yl)amino)-2-(3-((5-fluoro-2-methoxy-4- |
| | (methylsulfonyl)phenyl)amino)prop-1-yn-1-yl)-3-(2,2,2-trifluoroethyl)benzo[b]thiophene 1,1-dioxide; |
| 987 | 7-(((3R,4R)-3-fluoro-1-methylpiperidin-4-yl)amino)-2-(3-((5-fluoro-2-methoxy-4- |
| | (methylsulfonyl)phenyl)amino)prop-1-yn-1-yl)-3-(2,2,2-trifluoroethyl)benzo[b]thiophene 1,1-dioxide; |
| 988 | 7-(((3S,4S)-3-fluoro-1-methylpiperidin-4-yl)amino)-2-(3-((5-fluoro-2-methoxy-4- |
| | (methylsulfonyl)phenyl)amino)prop-1-yn-1-yl)-3-(2,2,2-trifluoroethyl)benzo[b]thiophene 1,1-dioxide; |
| 989 | 7-((4-(dimethylamino)cyclohexyl)amino)-2-(3-((5-fluoro-2-methoxy-4- |
| | (methylsulfonyl)phenyl)amino)prop-1-yn-1-yl)-3-(2,2,2-trifluoroethyl)benzo[b]thiophene 1,1-dioxide; |
| 990 | 7-((2-(dimethylamino)ethyl)amino)-2-(3-((5-fluoro-2-methoxy-4-(methylsulfonyl)phenyl)amino)prop-1-yn- |
| | 1-yl)-3-(2,2,2-trifluoroethyl)benzo[b]thiophene 1,1-dioxide; |
| 991 | 7-(((3S,4R)-3-fluoro-1-methylpiperidin-4-yl)amino)-2-(3-((5-fluoro-2-methoxy-4- |
| | (methylsulfonyl)phenyl)amino)prop-1-yn-1-yl)-3-(2,2,2-trifluoroethyl)benzo[b]thiophene 1,1-dioxide; |
| 992 | 2-(3-((5-fluoro-2-methoxy-4-(methylsulfonyl)phenyl)amino)prop-1-yn-1-yl)-7-((1-methylpiperidin-4- |
| | yl)amino)-3-(2,2,2-trifluoroethyl)benzo[b]thiophene 1,1-dioxide; |
| 993 | 2-(3-((5-fluoro-2-methoxy-4-(methylsulfonyl)phenyl)amino)prop-1-yn-1-yl)-7-((tetrahydro-2H-pyran-4- |
| | yl)amino)-3-(2,2,2-trifluoroethyl)benzo[b]thiophene 1,1-dioxide; |
| 994 | 3-(cyclopropylmethyl)-7-((3,3-difluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1,1-dioxide; |
| 995 | 3-(cyclopropylmethyl)-7-((1,1-dioxidotetrahydro-2H-thiopyran-4-yl)amino)-2-(3-((5-fluoro-2-methoxy-4- |
| | (methylsulfonyl)phenyl)amino)prop-1-yn-1-yl)benzo[b]thiophene 1,1-dioxide; |
| 996 | 3-(cyclopropylmethyl)-7-(((3R,4S)-3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1,1-dioxide; |
| 997 | 3-(cyclopropylmethyl)-7-((3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1,1-dioxide; |
| 998 | 1-(4-((3-(cyclopropylmethyl)-2-(3-((5-fluoro-2-methoxy-4-(methylsulfonyl)phenyl)amino)prop-1-yn-1-yl)- |
| | 1,1-dioxidobenzo[b]thiophen-7-yl)amino)piperidin-1-yl)-2-(dimethylamino)ethan-1-one; |
| 999 | 3-(cyclopropylmethyl)-7-((3-fluoro-1-(2-hydroxy-3-methoxypropyl)piperidin-4-yl)amino)-2-(3-((5-fluoro- |
| | 2-methoxy-4-(methylsulfonyl)phenyl)amino)prop-1-yn-1-yl)benzo[b]thiophene 1,1-dioxide; |
| 1000 | 3-(cyclopropylmethyl)-7-(((3R,4R)-3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1,1-dioxide; |
| 1001 | 3-(cyclopropylmethyl)-7-(((3S,4S)-3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1,1-dioxide; |
| 1002 | 3-(cyclopropylmethyl)-7-((4-(dimethylamino)cyclohexyl)amino)-2-(3-((5-fluoro-2-methoxy-4- |
| | (methylsulfonyl)phenyl)amino)prop-1-yn-1-yl)benzo[b]thiophene 1,1-dioxide; |
| 1003 | 3-(cyclopropylmethyl)-7-((2-(dimethylamino)ethyl)amino)-2-(3-((5-fluoro-2-methoxy-4- |
| | (methylsulfonyl)phenyl)amino)prop-1-yn-1-yl)benzo[b]thiophene 1,1-dioxide; |
| 1004 | 3-(cyclopropylmethyl)-7-(((3S,4R)-3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1,1-dioxide; |
| 1005 | 3-(cyclopropylmethyl)-2-(3-((5-fluoro-2-methoxy-4-(methylsulfonyl)phenyl)amino)prop-1-yn-1-yl)-7-((1- |
| | methylpiperidin-4-yl)amino)benzo[b]thiophene 1,1-dioxide; |
| 1006 | 3-(cyclopropylmethyl)-2-(3-((5-fluoro-2-methoxy-4-(methylsulfonyl)phenyl)amino)prop-1-yn-1-yl)-7- |
| | ((tetrahydro-2H-pyran-4-yl)amino)benzo[b]thiophene 1,1-dioxide; |
| 1007 | 7-((3,3-difluoro-1-methylpiperidin-4-yl)amino)-3-(2,2-difluoroethyl)-2-(3-((5-fluoro-2-methoxy-4- |
| | (methylsulfonyl)phenyl)amino)prop-1-yn-1-yl)benzo[b]thiophene 1,1-dioxide; |
| 1008 | 3-(2,2-difluoroethyl)-7-((1,1-dioxidotetrahydro-2H-thiopyran-4-yl)amino)-2-(3-((5-fluoro-2-methoxy-4- |
| | (methylsulfonyl)phenyl)amino)prop-1-yn-1-yl)benzo[b]thiophene 1,1-dioxide; |
| 1009 | 3-(2,2-difluoroethyl)-7-(((3R,4S)-3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1,1-dioxide; |
| 1010 | 3-(2,2-difluoroethyl)-7-((3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1,1-dioxide; |
| 1011 | 1-(4-((3-(2,2-difluoroethyl)-2-(3-((5-fluoro-2-methoxy-4-(methylsulfonyl)phenyl)amino)prop-1-yn-1-yl)- |
| | 1,1-dioxidobenzo[b]thiophen-7-yl)amino)piperidin-1-yl)-2-(dimethylamino)ethan-1-one; |
| 1012 | 3-(2,2-difluoroethyl)-7-((3-fluoro-1-(2-hydroxy-3-methoxypropyl)piperidin-4-yl)amino)-2-(3-((5-fluoro-2- |
| | methoxy-4-(methylsulfonyl)phenyl)amino)prop-1-yn-1-yl)benzo[b]thiophene 1,1-dioxide; |
| 1013 | 3-(2,2-difluoroethyl)-7-(((3R,4R)-3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1,1-dioxide; |
| 1014 | 3-(2,2-difluoroethyl)-7-(((3S,4S)-3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1,1-dioxide; |
| 1015 | 3-(2,2-difluoroethyl)-7-((4-(dimethylamino)cyclohexyl)amino)-2-(3-((5-fluoro-2-methoxy-4- |
| | (methylsulfonyl)phenyl)amino)prop-1-yn-1-yl)benzo[b]thiophene 1,1-dioxide; |
| 1016 | 3-(2,2-difluoroethyl)-7-((2-(dimethylamino)ethyl)amino)-2-(3-((5-fluoro-2-methoxy-4- |
| | (methylsulfonyl)phenyl)amino)prop-1-yn-1-yl)benzo[b]thiophene 1,1-dioxide; |
| 1017 | 3-(2,2-difluoroethyl)-7-(((3S,4R)-3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1,1-dioxide; |
| 1018 | 3-(2,2-difluoroethyl)-2-(3-((5-fluoro-2-methoxy-4-(methylsulfonyl)phenyl)amino)prop-1-yn-1-yl)-7-((1- |
| | methylpiperidin-4-yl)amino)benzo[b]thiophene 1,1-dioxide; |
| 1019 | 3-(2,2-difluoroethyl)-2-(3-((5-fluoro-2-methoxy-4-(methylsulfonyl)phenyl)amino)prop-1-yn-1-yl)-7- |
| | ((tetrahydro-2H-pyran-4-yl)amino)benzo[b]thiophene 1,1-dioxide; |
| 1020 | 7-((3,3-difluoro-1-methylpiperidin-4-yl)amino)-2-(3-((5-fluoro-2-methoxy-4- |
| | (methylsulfonyl)phenyl)amino)prop-1-yn-1-yl)-3-(1H-pyrrol-1-yl)benzo[b]thiophene 1,1-dioxide; |
| 1021 | 7-((1,1-dioxidotetrahydro-2H-thiopyran-4-yl)amino)-2-(3-((5-fluoro-2-methoxy-4- |
| | (methylsulfonyl)phenyl)amino)prop-1-yn-1-yl)-3-(1H-pyrrol-1-yl)benzo[b]thiophene 1,1-dioxide; |
| 1022 | 7-(((3R,4S)-3-fluoro-1-methylpiperidin-4-yl)amino)-2-(3-((5-fluoro-2-methoxy-4- |
| | (methylsulfonyl)phenyl)amino)prop-1-yn-1-yl)-3-(1H-pyrrol-1-yl)benzo[b]thiophene 1,1-dioxide; |
| 1023 | 7-((3-fluoro-1-methylpiperidin-4-yl)amino)-2-(3-((5-fluoro-2-methoxy-4- |
| | (methylsulfonyl)phenyl)amino)prop-1-yn-1-yl)-3-(1H-pyrrol-1-yl)benzo[b]thiophene 1,1-dioxide; |
| 1024 | 2-(dimethylamino)-1-(4-((2-(3-((5-fluoro-2-methoxy-4-(methylsulfonyl)phenyl)amino)prop-1-yn-1-yl)-1,1- |
| | dioxido-3-(1H-pyrrol-1-yl)benzo[b]thiophen-7-yl)amino)piperidin-1-yl)ethan-1-one; |
| 1025 | 7-((3-fluoro-1-(2-hydroxy-3-methoxypropyl)piperidin-4-yl)amino)-2-(3-((5-fluoro-2-methoxy-4- |
| | (methylsulfonyl)phenyl)amino)prop-1-yn-1-yl)-3-(1H-pyrrol-1-yl)benzo[b]thiophene 1,1-dioxide; |
| 1026 | 7-(((3R,4R)-3-fluoro-1-methylpiperidin-4-yl)amino)-2-(3-((5-fluoro-2-methoxy-4- |
| | (methylsulfonyl)phenyl)amino)prop-1-yn-1-yl)-3-(1H-pyrrol-1-yl)benzo[b]thiophene 1,1-dioxide; |
| 1027 | 7-(((3S,4S)-3-fluoro-1-methylpiperidin-4-yl)amino)-2-(3-((5-fluoro-2-methoxy-4- |
| | (methylsulfonyl)phenyl)amino)prop-1-yn-1-yl)-3-(1H-pyrrol-1-yl)benzo[b]thiophene 1,1-dioxide; |
| 1028 | 7-((4-(dimethylamino)cyclohexyl)amino)-2-(3-((5-fluoro-2-methoxy-4- |
| | (methylsulfonyl)phenyl)amino)prop-1-yn-1-yl)-3-(1H-pyrrol-1-yl)benzo[b]thiophene 1,1-dioxide; |
| 1029 | 7-((2-(dimethylamino)ethyl)amino)-2-(3-((5-fluoro-2-methoxy-4-(methylsulfonyl)phenyl)amino)prop-1-yn- |
| | 1-yl)-3-(1H-pyrrol-1-yl)benzo[b]thiophene 1,1-dioxide; |
| 1030 | 7-(((3S,4R)-3-fluoro-1-methylpiperidin-4-yl)amino)-2-(3-((5-fluoro-2-methoxy-4- |
| | (methylsulfonyl)phenyl)amino)prop-1-yn-1-yl)-3-(1H-pyrrol-1-yl)benzo[b]thiophene 1,1-dioxide; |
| 1031 | 2-(3-((5-fluoro-2-methoxy-4-(methylsulfonyl)phenyl)amino)prop-1-yn-1-yl)-7-((1-methylpiperidin-4- |
| | yl)amino)-3-(1H-pyrrol-1-yl)benzo[b]thiophene 1,1-dioxide; |
| 1032 | 2-(3-((5-fluoro-2-methoxy-4-(methylsulfonyl)phenyl)amino)prop-1-yn-1-yl)-3-(1H-pyrrol-1-yl)-7- |
| | ((tetrahydro-2H-pyran-4-yl)amino)benzo[b]thiophene 1,1-dioxide; |
| 1033 | 7-((3,3-difluoro-1-methylpiperidin-4-yl)amino)-3-ethyl-2-(3-((5-fluoro-2-methoxy-4- |
| | (methylsulfonyl)phenyl)amino)prop-1-yn-1-yl)benzo[b]thiophene 1,1-dioxide; |
| 1034 | 7-((1,1-dioxidotetrahydro-2H-thiopyran-4-yl)amino)-3-ethyl-2-(3-((5-fluoro-2-methoxy-4- |
| | (methylsulfonyl)phenyl)amino)prop-1-yn-1-yl)benzo[b]thiophene 1,1-dioxide; |
| 1035 | 3-ethyl-7-(((3R,4S)-3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1,1-dioxide; |
| 1036 | 3-ethyl-7-((3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1,1-dioxide; |
| 1037 | 2-(dimethylamino)-1-(4-((3-ethyl-2-(3-((5-fluoro-2-methoxy-4-(methylsulfonyl)phenyl)amino)prop-1-yn-1- |
| | yl)-1,1-dioxidobenzo[b]thiophen-7-yl)amino)piperidin-1-yl)ethan-1-one; |
| 1038 | 3-ethyl-7-((3-fluoro-1-(2-hydroxy-3-methoxypropyl)piperidin-4-yl)amino)-2-(3-((5-fluoro-2-methoxy-4- |
| | (methylsulfonyl)phenyl)amino)prop-1-yn-1-yl)benzo[b]thiophene 1,1-dioxide; |
| 1039 | 3-ethyl-7-(((3R,4R)-3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1,1-dioxide; |
| 1040 | 3-ethyl-7-(((3S,4S)-3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1,1-dioxide; |
| 1041 | 7-((4-(dimethylamino)cyclohexyl)amino)-3-ethyl-2-(3-((5-fluoro-2-methoxy-4- |
| | (methylsulfonyl)phenyl)amino)prop-1-yn-1-yl)benzo[b]thiophene 1,1-dioxide; |
| 1042 | 7-((2-(dimethylamino)ethyl)amino)-3-ethyl-2-(3-((5-fluoro-2-methoxy-4- |
| | (methylsulfonyl)phenyl)amino)prop-1-yn-1-yl)benzo[b]thiophene 1,1-dioxide; |
| 1043 | 3-ethyl-7-(((3S,4R)-3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1,1-dioxide; |
| 1044 | 3-ethyl-2-(3-((5-fluoro-2-methoxy-4-(methylsulfonyl)phenyl)amino)prop-1-yn-1-yl)-7-((1-methylpiperidin- |
| | 4-yl)amino)benzo[b]thiophene 1,1-dioxide; |
| 1045 | 3-ethyl-2-(3-((5-fluoro-2-methoxy-4-(methylsulfonyl)phenyl)amino)prop-1-yn-1-yl)-7-((tetrahydro-2H- |
| | pyran-4-yl)amino)benzo[b]thiophene 1,1-dioxide; |
| 1046 | 4-((3-(3-(cyanomethyl)-7-((3,3-difluoro-1-methylpiperidin-4-yl)amino)-1,1-dioxidobenzo[b]thiophen-2- |
| | yl)prop-2-yn-1-yl)amino)-3-methoxy-N-methylbenzamide; |
| 1047 | 4-((3-(3-(cyanomethyl)-7-((1,1-dioxidotetrahydro-2H-thiopyran-4-yl)amino)-1,1-dioxidobenzo[b]thiophen- |
| | 2-yl)prop-2-yn-1-yl)amino)-3-methoxy-N-methylbenzamide; |
| 1048 | 4-((3-(3-(cyanomethyl)-7-(((3R,4S)-3-fluoro-1-methylpiperidin-4-yl)amino)-1,1-dioxidobenzo[b]thiophen- |
| | 2-yl)prop-2-yn-1-yl)amino)-3-methoxy-N-methylbenzamide; |
| 1049 | 4-((3-(3-(cyanomethyl)-7-((3-fluoro-1-methylpiperidin-4-yl)amino)-1,1-dioxidobenzo[b]thiophen-2- |
| | yl)prop-2-yn-1-yl)amino)-3-methoxy-N-methylbenzamide; |
| 1050 | 4-((3-(3-(cyanomethyl)-7-((1-(dimethylglycyl)piperidin-4-yl)amino)-1,1-dioxidobenzo[b]thiophen-2- |
| | yl)prop-2-yn-1-yl)amino)-3-methoxy-N-methylbenzamide; |
| 1051 | 4-((3-(3-(cyanomethyl)-7-((3-fluoro-1-(2-hydroxy-3-methoxypropyl)piperidin-4-yl)amino)-1,1- |
| | dioxidobenzo[b]thiophen-2-yl)prop-2-yn-1-yl)amino)-3-methoxy-N-methylbenzamide; |
| 1052 | 4-((3-(3-(cyanomethyl)-7-(((3R,4R)-3-fluoro-1-methylpiperidin-4-yl)amino)-1,1-dioxidobenzo[b]thiophen- |
| | 2-yl)prop-2-yn-1-yl)amino)-3-methoxy-N-methylbenzamide; |
| 1053 | 4-((3-(3-(cyanomethyl)-7-(((3S,4S)-3-fluoro-1-methylpiperidin-4-yl)amino)-1,1-dioxidobenzo[b]thiophen- |
| | 2-yl)prop-2-yn-1-yl)amino)-3-methoxy-N-methylbenzamide; |
| 1054 | 4-((3-(3-(cyanomethyl)-7-((4-(dimethylamino)cyclohexyl)amino)-1,1-dioxidobenzo[b]thiophen-2-yl)prop- |
| | 2-yn-1-yl)amino)-3-methoxy-N-methylbenzamide; |
| 1055 | 4-((3-(3-(cyanomethyl)-7-((2-(dimethylamino)ethyl)amino)-1,1-dioxidobenzo[b]thiophen-2-yl)prop-2-yn-1- |
| | yl)amino)-3-methoxy-N-methylbenzamide; |
| 1056 | 4-((3-(3-(cyanomethyl)-7-(((3S,4R)-3-fluoro-1-methylpiperidin-4-yl)amino)-1,1-dioxidobenzo[b]thiophen- |
| | 2-yl)prop-2-yn-1-yl)amino)-3-methoxy-N-methylbenzamide; |
| 1057 | 4-((3-(3-(cyanomethyl)-7-((1-methylpiperidin-4-yl)amino)-1,1-dioxidobenzo[b]thiophen-2-yl)prop-2-yn-1- |
| | yl)amino)-3-methoxy-N-methylbenzamide; |
| 1058 | 4-((3-(3-(cyanomethyl)-1,1-dioxido-7-((tetrahydro-2H-pyran-4-yl)amino)benzo[b]thiophen-2-yl)prop-2-yn- |
| | 1-yl)amino)-3-methoxy-N-methylbenzamide; |
| 1059 | 4-((3-(7-((3,3-difluoro-1-methylpiperidin-4-yl)amino)-1,1-dioxido-3-(thiazol-4-yl)benzo[b]thiophen-2- |
| | yl)prop-2-yn-1-yl)amino)-3-methoxy-N-methylbenzamide; |
| 1060 | 4-((3-(7-((1,1-dioxidotetrahydro-2H-thiopyran-4-yl)amino)-1,1-dioxido-3-(thiazol-4-yl)benzo[b]thiophen- |
| | 2-yl)prop-2-yn-1-yl)amino)-3-methoxy-N-methylbenzamide; |
| 1061 | 4-((3-(7-(((3R,4S)-3-fluoro-1-methylpiperidin-4-yl)amino)-1,1-dioxido-3-(thiazol-4-yl)benzo[b]thiophen-2- |
| | yl)prop-2-yn-1-yl)amino)-3-methoxy-N-methylbenzamide; |
| 1062 | 4-((3-(7-((3-fluoro-1-methylpiperidin-4-yl)amino)-1,1-dioxido-3-(thiazol-4-yl)benzo[b]thiophen-2-yl)prop- |
| | 2-yn-1-yl)amino)-3-methoxy-N-methylbenzamide; |
| 1063 | 4-((3-(7-((1-(dimethylglycyl)piperidin-4-yl)amino)-1,1-dioxido-3-(thiazol-4-yl)benzo[b]thiophen-2- |
| | yl)prop-2-yn-1-yl)amino)-3-methoxy-N-methylbenzamide; |
| 1064 | 4-((3-(7-((3-fluoro-1-(2-hydroxy-3-methoxypropyl)piperidin-4-yl)amino)-1,1-dioxido-3-(thiazol-4- |
| | yl)benzo[b]thiophen-2-yl)prop-2-yn-1-yl)amino)-3-methoxy-N-methylbenzamide; |
| 1065 | 4-((3-(7-(((3R,4R)-3-fluoro-1-methylpiperidin-4-yl)amino)-1,1-dioxido-3-(thiazol-4-yl)benzo[b]thiophen-2- |
| | yl)prop-2-yn-1-yl)amino)-3-methoxy-N-methylbenzamide; |
| 1066 | 4-((3-(7-(((3S,4S)-3-fluoro-1-methylpiperidin-4-yl)amino)-1,1-dioxido-3-(thiazol-4-yl)benzo[b]thiophen-2- |
| | yl)prop-2-yn-1-yl)amino)-3-methoxy-N-methylbenzamide; |
| 1067 | 4-((3-(7-((4-(dimethylamino)cyclohexyl)amino)-1,1-dioxido-3-(thiazol-4-yl)benzo[b]thiophen-2-yl)prop-2- |
| | yn-1-yl)amino)-3-methoxy-N-methylbenzamide; |
| 1068 | 4-((3-(7-((2-(dimethylamino)ethyl)amino)-1,1-dioxido-3-(thiazol-4-yl)benzo[b]thiophen-2-yl)prop-2-yn-1- |
| | yl)amino)-3-methoxy-N-methylbenzamide; |
| 1069 | 4-((3-(7-(((3S,4R)-3-fluoro-1-methylpiperidin-4-yl)amino)-1,1-dioxido-3-(thiazol-4-yl)benzo[b]thiophen-2- |
| | yl)prop-2-yn-1-yl)amino)-3-methoxy-N-methylbenzamide; |
| 1070 | 3-methoxy-N-methyl-4-((3-(7-((1-methylpiperidin-4-yl)amino)-1,1-dioxido-3-(thiazol-4- |
| | yl)benzo[b]thiophen-2-yl)prop-2-yn-1-yl)amino)benzamide; |
| 1071 | 4-((3-(1,1-dioxido-7-((tetrahydro-2H-pyran-4-yl)amino)-3-(thiazol-4-yl)benzo[b]thiophen-2-yl)prop-2-yn- |
| | 1-yl)amino)-3-methoxy-N-methylbenzamide; |
| 1072 | 4-((3-(7-((3,3-difluoro-1-methylpiperidin-4-yl)amino)-1,1-dioxido-3-(2,2,2- |
| | trifluoroethyl)benzo[b]thiophen-2-yl)prop-2-yn-1-yl)amino)-3-methoxy-N-methylbenzamide; |
| 1073 | 4-((3-(7-((1,1-dioxidotetrahydro-2H-thiopyran-4-yl)amino)-1,1-dioxido-3-(2,2,2- |
| | trifluoroethyl)benzo[b]thiophen-2-yl)prop-2-yn-1-yl)amino)-3-methoxy-N-methylbenzamide; |
| 1074 | 4-((3-(7-(((3R,4S)-3-fluoro-1-methylpiperidin-4-yl)amino)-1,1-dioxido-3-(2,2,2- |
| | trifluoroethyl)benzo[b]thiophen-2-yl)prop-2-yn-1-yl)amino)-3-methoxy-N-methylbenzamide; |
| 1075 | 4-((3-(7-((3-fluoro-1-methylpiperidin-4-yl)amino)-1,1-dioxido-3-(2,2,2-trifluoroethyl)benzo[b]thiophen-2- |
| | yl)prop-2-yn-1-yl)amino)-3-methoxy-N-methylbenzamide; |
| 1076 | 4-((3-(7-((1-(dimethylglycyl)piperidin-4-yl)amino)-1,1-dioxido-3-(2,2,2-trifluoroethyl)benzo[b]thiophen-2- |
| | yl)prop-2-yn-1-yl)amino)-3-methoxy-N-methylbenzamide; |
| 1077 | 4-((3-(7-((3-fluoro-1-(2-hydroxy-3-methoxypropyl)piperidin-4-yl)amino)-1,1-dioxido-3-(2,2,2- |
| | trifluoroethyl)benzo[b]thiophen-2-yl)prop-2-yn-1-yl)amino)-3-methoxy-N-methylbenzamide; |
| 1078 | 4-((3-(7-(((3R,4R)-3-fluoro-1-methylpiperidin-4-yl)amino)-1,1-dioxido-3-(2,2,2- |
| | trifluoroethyl)benzo[b]thiophen-2-yl)prop-2-yn-1-yl)amino)-3-methoxy-N-methylbenzamide; |
| 1079 | 4-((3-(7-(((3S,4S)-3-fluoro-1-methylpiperidin-4-yl)amino)-1,1-dioxido-3-(2,2,2- |
| | trifluoroethyl)benzo[b]thiophen-2-yl)prop-2-yn-1-yl)amino)-3-methoxy-N-methylbenzamide; |
| 1080 | 4-((3-(7-((4-(dimethylamino)cyclohexyl)amino)-1,1-dioxido-3-(2,2,2-trifluoroethyl)benzo[b]thiophen-2- |
| | yl)prop-2-yn-1-yl)amino)-3-methoxy-N-methylbenzamide; |
| 1081 | 4-((3-(7-((2-(dimethylamino)ethyl)amino)-1,1-dioxido-3-(2,2,2-trifluoroethyl)benzo[b]thiophen-2-yl)prop- |
| | 2-yn-1-yl)amino)-3-methoxy-N-methylbenzamide; |
| 1082 | 4-((3-(7-(((3S,4R)-3-fluoro-1-methylpiperidin-4-yl)amino)-1,1-dioxido-3-(2,2,2- |
| | trifluoroethyl)benzo[b]thiophen-2-yl)prop-2-yn-1-yl)amino)-3-methoxy-N-methylbenzamide; |
| 1083 | 3-methoxy-N-methyl-4-((3-(7-((1-methylpiperidin-4-yl)amino)-1,1-dioxido-3-(2,2,2- |
| | trifluoroethyl)benzo[b]thiophen-2-yl)prop-2-yn-1-yl)amino)benzamide; |
| 1084 | 4-((3-(1,1-dioxido-7-((tetrahydro-2H-pyran-4-yl)amino)-3-(2,2,2-trifluoroethyl)benzo[b]thiophen-2- |
| | yl)prop-2-yn-1-yl)amino)-3-methoxy-N-methylbenzamide; |
| 1085 | 4-((3-(3-(cyclopropylmethyl)-7-((3,3-difluoro-1-methylpiperidin-4-yl)amino)-1,1-dioxidobenzo[b]thiophen- |
| | 2-yl)prop-2-yn-1-yl)amino)-3-methoxy-N-methylbenzamide; |
| 1086 | 4-((3-(3-(cyclopropylmethyl)-7-((1,1-dioxidotetrahydro-2H-thiopyran-4-yl)amino)-1,1- |
| | dioxidobenzo[b]thiophen-2-yl)prop-2-yn-1-yl)amino)-3-methoxy-N-methylbenzamide; |
| 1087 | 4-((3-(3-(cyclopropylmethyl)-7-(((3R,4S)-3-fluoro-1-methylpiperidin-4-yl)amino)-1,1- |
| | dioxidobenzo[b]thiophen-2-yl)prop-2-yn-1-yl)amino)-3-methoxy-N-methylbenzamide; |
| 1088 | 4-((3-(3-(cyclopropylmethyl)-7-((3-fluoro-1-methylpiperidin-4-yl)amino)-1,1-dioxidobenzo[b]thiophen-2- |
| | yl)prop-2-yn-1-yl)amino)-3-methoxy-N-methylbenzamide; |
| 1089 | 4-((3-(3-(cyclopropylmethyl)-7-((1-(dimethylglycyl)piperidin-4-yl)amino)-1,1-dioxidobenzo[b]thiophen-2- |
| | yl)prop-2-yn-1-yl)amino)-3-methoxy-N-methylbenzamide; |
| 1090 | 4-((3-(3-(cyclopropylmethyl)-7-((3-fluoro-1-(2-hydroxy-3-methoxypropyl)piperidin-4-yl)amino)-1,1- |
| | dioxidobenzo[b]thiophen-2-yl)prop-2-yn-1-yl)amino)-3-methoxy-N-methylbenzamide; |
| 1091 | 4-((3-(3-(cyclopropylmethyl)-7-(((3R,4R)-3-fluoro-1-methylpiperidin-4-yl)amino)-1,1- |
| | dioxidobenzo[b]thiophen-2-yl)prop-2-yn-1-yl)amino)-3-methoxy-N-methylbenzamide; |
| 1092 | 4-((3-(3-(cyclopropylmethyl)-7-(((3S,4S)-3-fluoro-1-methylpiperidin-4-yl)amino)-1,1- |
| | dioxidobenzo[b]thiophen-2-yl)prop-2-yn-1-yl)amino)-3-methoxy-N-methylbenzamide; |
| 1093 | 4-((3-(3-(cyclopropylmethyl)-7-((4-(dimethylamino)cyclohexyl)amino)-1,1-dioxidobenzo[b]thiophen-2- |
| | yl)prop-2-yn-1-yl)amino)-3-methoxy-N-methylbenzamide; |
| 1094 | 4-((3-(3-(cyclopropylmethyl)-7-((2-(dimethylamino)ethyl)amino)-1,1-dioxidobenzo[b]thiophen-2-yl)prop- |
| | 2-yn-1-yl)amino)-3-methoxy-N-methylbenzamide; |
| 1095 | 4-((3-(3-(cyclopropylmethyl)-7-(((3S,4R)-3-fluoro-1-methylpiperidin-4-yl)amino)-1,1- |
| | dioxidobenzo[b]thiophen-2-yl)prop-2-yn-1-yl)amino)-3-methoxy-N-methylbenzamide; |
| 1096 | 4-((3-(3-(cyclopropylmethyl)-7-((1-methylpiperidin-4-yl)amino)-1,1-dioxidobenzo[b]thiophen-2-yl)prop-2- |
| | yn-1-yl)amino)-3-methoxy-N-methylbenzamide; |
| 1097 | 4-((3-(3-(cyclopropylmethyl)-1,1-dioxido-7-((tetrahydro-2H-pyran-4-yl)amino)benzo[b]thiophen-2- |
| | yl)prop-2-yn-1-yl)amino)-3-methoxy-N-methylbenzamide; |
| 1098 | 4-((3-(7-((3,3-difluoro-1-methylpiperidin-4-yl)amino)-3-(2,2-difluoroethyl)-1,1-dioxidobenzo[b]thiophen- |
| | 2-yl)prop-2-yn-1-yl)amino)-3-methoxy-N-methylbenzamide; |
| 1099 | 4-((3-(3-(2,2-difluoroethyl)-7-((1,1-dioxidotetrahydro-2H-thiopyran-4-yl)amino)-1,1- |
| | dioxidobenzo[b]thiophen-2-yl)prop-2-yn-1-yl)amino)-3-methoxy-N-methylbenzamide; |
| 1100 | 4-((3-(3-(2,2-difluoroethyl)-7-(((3R,4S)-3-fluoro-1-methylpiperidin-4-yl)amino)-1,1- |
| | dioxidobenzo[b]thiophen-2-yl)prop-2-yn-1-yl)amino)-3-methoxy-N-methylbenzamide; |
| 1101 | 4-((3-(3-(2,2-difluoroethyl)-7-((3-fluoro-1-methylpiperidin-4-yl)amino)-1,1-dioxidobenzo[b]thiophen-2- |
| | yl)prop-2-yn-1-yl)amino)-3-methoxy-N-methylbenzamide; |
| 1102 | 4-((3-(3-(2,2-difluoroethyl)-7-((1-(dimethylglycyl)piperidin-4-yl)amino)-1,1-dioxidobenzo[b]thiophen-2- |
| | yl)prop-2-yn-1-yl)amino)-3-methoxy-N-methylbenzamide; |
| 1103 | 4-((3-(3-(2,2-difluoroethyl)-7-((3-fluoro-1-(2-hydroxy-3-methoxypropyl)piperidin-4-yl)amino)-1,1- |
| | dioxidobenzo[b]thiophen-2-yl)prop-2-yn-1-yl)amino)-3-methoxy-N-methylbenzamide; |
| 1104 | 4-((3-(3-(2,2-difluoroethyl)-7-(((3R,4R)-3-fluoro-1-methylpiperidin-4-yl)amino)-1,1- |
| | dioxidobenzo[b]thiophen-2-yl)prop-2-yn-1-yl)amino)-3-methoxy-N-methylbenzamide; |
| 1105 | 4-((3-(3-(2,2-difluoroethyl)-7-(((3S,4S)-3-fluoro-1-methylpiperidin-4-yl)amino)-1,1- |
| | dioxidobenzo[b]thiophen-2-yl)prop-2-yn-1-yl)amino)-3-methoxy-N-methylbenzamide; |
| 1106 | 4-((3-(3-(2,2-difluoroethyl)-7-((4-(dimethylamino)cyclohexyl)amino)-1,1-dioxidobenzo[b]thiophen-2- |
| | yl)prop-2-yn-1-yl)amino)-3-methoxy-N-methylbenzamide; |
| 1107 | 4-((3-(3-(2,2-difluoroethyl)-7-((2-(dimethylamino)ethyl)amino)-1,1-dioxidobenzo[b]thiophen-2-yl)prop-2- |
| | yn-1-yl)amino)-3-methoxy-N-methylbenzamide; |
| 1108 | 4-((3-(3-(2,2-difluoroethyl)-7-(((3S,4R)-3-fluoro-1-methylpiperidin-4-yl)amino)-1,1- |
| | dioxidobenzo[b]thiophen-2-yl)prop-2-yn-1-yl)amino)-3-methoxy-N-methylbenzamide; |
| 1109 | 4-((3-(3-(2,2-difluoroethyl)-7-((1-methylpiperidin-4-yl)amino)-1,1-dioxidobenzo[b]thiophen-2-yl)prop-2- |
| | yn-1-yl)amino)-3-methoxy-N-methylbenzamide; |
| 1110 | 4-((3-(3-(2,2-difluoroethyl)-1,1-dioxido-7-((tetrahydro-2H-pyran-4-yl)amino)benzo[b]thiophen-2-yl)prop- |
| | 2-yn-1-yl)amino)-3-methoxy-N-methylbenzamide; |
| 1111 | 4-((3-(7-((3,3-difluoro-1-methylpiperidin-4-yl)amino)-1,1-dioxido-3-(1H-pyrrol-1-yl)benzo[b]thiophen-2- |
| | yl)prop-2-yn-1-yl)amino)-3-methoxy-N-methylbenzamide; |
| 1112 | 4-((3-(7-((1,1-dioxidotetrahydro-2H-thiopyran-4-yl)amino)-1,1-dioxido-3-(1H-pyrrol-1- |
| | yl)benzo[b]thiophen-2-yl)prop-2-yn-1-yl)amino)-3-methoxy-N-methylbenzamide; |
| 1113 | 4-((3-(7-(((3R,4S)-3-fluoro-1-methylpiperidin-4-yl)amino)-1,1-dioxido-3-(1H-pyrrol-1- |
| | yl)benzo[b]thiophen-2-yl)prop-2-yn-1-yl)amino)-3-methoxy-N-methylbenzamide; |
| 1114 | 4-((3-(7-((3-fluoro-1-methylpiperidin-4-yl)amino)-1,1-dioxido-3-(1H-pyrrol-1-yl)benzo[b]thiophen-2- |
| | yl)prop-2-yn-1-yl)amino)-3-methoxy-N-methylbenzamide; |
| 1115 | 4-((3-(7-((1-(dimethylglycyl)piperidin-4-yl)amino)-1,1-dioxido-3-(1H-pyrrol-1-yl)benzo[b]thiophen-2- |
| | yl)prop-2-yn-1-yl)amino)-3-methoxy-N-methylbenzamide; |
| 1116 | 4-((3-(7-((3-fluoro-1-(2-hydroxy-3-methoxypropyl)piperidin-4-yl)amino)-1,1-dioxido-3-(1H-pyrrol-1- |
| | yl)benzo[b]thiophen-2-yl)prop-2-yn-1-yl)amino)-3-methoxy-N-methylbenzamide; |
| 1117 | 4-((3-(7-(((3R,4R)-3-fluoro-1-methylpiperidin-4-yl)amino)-1,1-dioxido-3-(1H-pyrrol-1- |
| | yl)benzo[b]thiophen-2-yl)prop-2-yn-1-yl)amino)-3-methoxy-N-methylbenzamide; |
| 1118 | 4-((3-(7-(((3S,4S)-3-fluoro-1-methylpiperidin-4-yl)amino)-1,1-dioxido-3-(1H-pyrrol-1- |
| | yl)benzo[b]thiophen-2-yl)prop-2-yn-1-yl)amino)-3-methoxy-N-methylbenzamide; |
| 1119 | 4-((3-(7-((4-(dimethylamino)cyclohexyl)amino)-1,1-dioxido-3-(1H-pyrrol-1-yl)benzo[b]thiophen-2- |
| | yl)prop-2-yn-1-yl)amino)-3-methoxy-N-methylbenzamide; |
| 1120 | 4-((3-(7-((2-(dimethylamino)ethyl)amino)-1,1-dioxido-3-(1H-pyrrol-1-yl)benzo[b]thiophen-2-yl)prop-2-yn- |
| | 1-yl)amino)-3-methoxy-N-methylbenzamide; |
| 1121 | 4-((3-(7-(((3S,4R)-3-fluoro-1-methylpiperidin-4-yl)amino)-1,1-dioxido-3-(1H-pyrrol-1- |
| | yl)benzo[b]thiophen-2-yl)prop-2-yn-1-yl)amino)-3-methoxy-N-methylbenzamide; |
| 1122 | 3-methoxy-N-methyl-4-((3-(7-((1-methylpiperidin-4-yl)amino)-1,1-dioxido-3-(1H-pyrrol-1- |
| | yl)benzo[b]thiophen-2-yl)prop-2-yn-1-yl)amino)benzamide; |
| 1123 | 4-((3-(1,1-dioxido-3-(1H-pyrrol-1-yl)-7-((tetrahydro-2H-pyran-4-yl)amino)benzo[b]thiophen-2-yl)prop-2- |
| | yn-1-yl)amino)-3-methoxy-N-methylbenzamide; |
| 1124 | 4-((3-(7-((3,3-difluoro-1-methylpiperidin-4-yl)amino)-3-ethyl-1,1-dioxidobenzo[b]thiophen-2-yl)prop-2- |
| | yn-1-yl)amino)-3-methoxy-N-methylbenzamide; |
| 1125 | 4-((3-(7-((1,1-dioxidotetrahydro-2H-thiopyran-4-yl)amino)-3-ethyl-1,1-dioxidobenzo[b]thiophen-2-yl)prop- |
| | 2-yn-1-yl)amino)-3-methoxy-N-methylbenzamide; |
| 1126 | 4-((3-(3-ethyl-7-(((3R,4S)-3-fluoro-1-methylpiperidin-4-yl)amino)-1,1-dioxidobenzo[b]thiophen-2-yl)prop- |
| | 2-yn-1-yl)amino)-3-methoxy-N-methylbenzamide; |
| 1127 | 4-((3-(3-ethyl-7-((3-fluoro-1-methylpiperidin-4-yl)amino)-1,1-dioxidobenzo[b]thiophen-2-yl)prop-2-yn-1- |
| | yl)amino)-3-methoxy-N-methylbenzamide; |
| 1128 | 4-((3-(7-((1-(dimethylglycyl)piperidin-4-yl)amino)-3-ethyl-1,1-dioxidobenzo[b]thiophen-2-yl)prop-2-yn-1- |
| | yl)amino)-3-methoxy-N-methylbenzamide; |
| 1129 | 4-((3-(3-ethyl-7-((3-fluoro-1-(2-hydroxy-3-methoxypropyl)piperidin-4-yl)amino)-1,1- |
| | dioxidobenzo[b]thiophen-2-yl)prop-2-yn-1-yl)amino)-3-methoxy-N-methylbenzamide; |
| 1130 | 4-((3-(3-ethyl-7-(((3R,4R)-3-fluoro-1-methylpiperidin-4-yl)amino)-1,1-dioxidobenzo[b]thiophen-2-yl)prop- |
| | 2-yn-1-yl)amino)-3-methoxy-N-methylbenzamide; |
| 1131 | 4-((3-(3-ethyl-7-(((3S,4S)-3-fluoro-1-methylpiperidin-4-yl)amino)-1,1-dioxidobenzo[b]thiophen-2-yl)prop- |
| | 2-yn-1-yl)amino)-3-methoxy-N-methylbenzamide; |
| 1132 | 4-((3-(7-((4-(dimethylamino)cyclohexyl)amino)-3-ethyl-1,1-dioxidobenzo[b]thiophen-2-yl)prop-2-yn-1- |
| | yl)amino)-3-methoxy-N-methylbenzamide; |
| 1133 | 4-((3-(7-((2-(dimethylamino)ethyl)amino)-3-ethyl-1,1-dioxidobenzo[b]thiophen-2-yl)prop-2-yn-1- |
| | yl)amino)-3-methoxy-N-methylbenzamide; |
| 1134 | 4-((3-(3-ethyl-7-(((3S,4R)-3-fluoro-1-methylpiperidin-4-yl)amino)-1,1-dioxidobenzo[b]thiophen-2-yl)prop- |
| | 2-yn-1-yl)amino)-3-methoxy-N-methylbenzamide; |
| 1135 | 4-((3-(3-ethyl-7-((1-methylpiperidin-4-yl)amino)-1,1-dioxidobenzo[b]thiophen-2-yl)prop-2-yn-1- |
| | yl)amino)-3-methoxy-N-methylbenzamide; |
| 1136 | 4-((3-(3-ethyl-1,1-dioxido-7-((tetrahydro-2H-pyran-4-yl)amino)benzo[b]thiophen-2-yl)prop-2-yn-1- |
| | yl)amino)-3-methoxy-N-methylbenzamide; |
| 1137 | 2-(7-((3,3-difluoro-1-methylpiperidin-4-yl)amino)-2-(3-((2-methoxy-4- |
| | (methylsulfonyl)phenyl)amino)prop-1-yn-1-yl)-1,1-dioxidobenzo[b]thiophen-3-yl)acetonitrile; |
| 1138 | 2-(7-((1,1-dioxidotetrahydro-2H-thiopyran-4-yl)amino)-2-(3-((2-methoxy-4- |
| | (methylsulfonyl)phenyl)amino)prop-1-yn-1-yl)-1,1-dioxidobenzo[b]thiophen-3-yl)acetonitrile; |
| 1139 | 2-(7-(((3R,4S)-3-fluoro-1-methylpiperidin-4-yl)amino)-2-(3-((2-methoxy-4- |
| | (methylsulfonyl)phenyl)amino)prop-1-yn-1-yl)-1,1-dioxidobenzo[b]thiophen-3-yl)acetonitrile; |
| 1140 | 2-(7-((3-fluoro-1-methylpiperidin-4-yl)amino)-2-(3-((2-methoxy-4-(methylsulfonyl)phenyl)amino)prop-1- |
| | yn-1-yl)-1,1-dioxidobenzo[b]thiophen-3-yl)acetonitrile; |
| 1141 | 2-(7-((1-(dimethylglycyl)piperidin-4-yl)amino)-2-(3-((2-methoxy-4-(methylsulfonyl)phenyl)amino)prop-1- |
| | yn-1-yl)-1,1-dioxidobenzo[b]thiophen-3-yl)acetonitrile; |
| 1142 | 2-(7-((3-fluoro-1-(2-hydroxy-3-methoxypropyl)piperidin-4-yl)amino)-2-(3-((2-methoxy-4- |
| | (methylsulfonyl)phenyl)amino)prop-1-yn-1-yl)-1,1-dioxidobenzo[b]thiophen-3-yl)acetonitrile; |
| 1143 | 2-(7-(((3R,4R)-3-fluoro-1-methylpiperidin-4-yl)amino)-2-(3-((2-methoxy-4- |
| | (methylsulfonyl)phenyl)amino)prop-1-yn-1-yl)-1,1-dioxidobenzo[b]thiophen-3-yl)acetonitrile; |
| 1144 | 2-(7-(((3S,4S)-3-fluoro-1-methylpiperidin-4-yl)amino)-2-(3-((2-methoxy-4- |
| | (methylsulfonyl)phenyl)amino)prop-1-yn-1-yl)-1,1-dioxidobenzo[b]thiophen-3-yl)acetonitrile; |
| 1145 | 2-(7-((4-(dimethylamino)cyclohexyl)amino)-2-(3-((2-methoxy-4-(methylsulfonyl)phenyl)amino)prop-1-yn- |
| | 1-yl)-1,1-dioxidobenzo[b]thiophen-3-yl)acetonitrile; |
| 1146 | 2-(7-((2-(dimethylamino)ethyl)amino)-2-(3-((2-methoxy-4-(methylsulfonyl)phenyl)amino)prop-1-yn-1-yl)- |
| | 1,1-dioxidobenzo[b]thiophen-3-yl)acetonitrile; |
| 1147 | 2-(7-(((3S,4R)-3-fluoro-1-methylpiperidin-4-yl)amino)-2-(3-((2-methoxy-4- |
| | (methylsulfonyl)phenyl)amino)prop-1-yn-1-yl)-1,1-dioxidobenzo[b]thiophen-3-yl)acetonitrile; |
| 1148 | 2-(2-(3-((2-methoxy-4-(methylsulfonyl)phenyl)amino)prop-1-yn-1-yl)-7-((1-methylpiperidin-4-yl)amino)- |
| | 1,1-dioxidobenzo[b]thiophen-3-yl)acetonitrile; |
| 1149 | 2-(2-(3-((2-methoxy-4-(methylsulfonyl)phenyl)amino)prop-1-yn-1-yl)-1,1-dioxido-7-((tetrahydro-2H- |
| | pyran-4-yl)amino)benzo[b]thiophen-3-yl)acetonitrile; |
| 1150 | 7-((3,3-difluoro-1-methylpiperidin-4-yl)amino)-2-(3-((2-methoxy-4-(methylsulfonyl)phenyl)amino)prop-1- |
| | yn-1-yl)-3-(thiazol-4-yl)benzo[b]thiophene 1,1-dioxide; |
| 1151 | 7-((1,1-dioxidotetrahydro-2H-thiopyran-4-yl)amino)-2-(3-((2-methoxy-4- |
| | (methylsulfonyl)phenyl)amino)prop-1-yn-1-yl)-3-(thiazol-4-yl)benzo[b]thiophene 1,1-dioxide; |
| 1152 | 7-(((3R,4S)-3-fluoro-1-methylpiperidin-4-yl)amino)-2-(3-((2-methoxy-4- |
| | (methylsulfonyl)phenyl)amino)prop-1-yn-1-yl)-3-(thiazol-4-yl)benzo[b]thiophene 1,1-dioxide; |
| 1153 | 7-((3-fluoro-1-methylpiperidin-4-yl)amino)-2-(3-((2-methoxy-4-(methylsulfonyl)phenyl)amino)prop-1-yn- |
| | 1-yl)-3-(thiazol-4-yl)benzo[b]thiophene 1,1-dioxide; |
| 1154 | 2-(dimethylamino)-1-(4-((2-(3-((2-methoxy-4-(methylsulfonyl)phenyl)amino)prop-1-yn-1-yl)-1,1-dioxido- |
| | 3-(thiazol-4-yl)benzo[b]thiophen-7-yl)amino)piperidin-1-yl)ethan-1-one; |
| 1155 | 7-((3-fluoro-1-(2-hydroxy-3-methoxypropyl)piperidin-4-yl)amino)-2-(3-((2-methoxy-4- |
| | (methylsulfonyl)phenyl)amino)prop-1-yn-1-yl)-3-(thiazol-4-yl)benzo[b]thiophene 1,1-dioxide; |
| 1156 | 7-(((3R,4R)-3-fluoro-1-methylpiperidin-4-yl)amino)-2-(3-((2-methoxy-4- |
| | (methylsulfonyl)phenyl)amino)prop-1-yn-1-yl)-3-(thiazol-4-yl)benzo[b]thiophene 1,1-dioxide; |
| 1157 | 7-(((3S,4S)-3-fluoro-1-methylpiperidin-4-yl)amino)-2-(3-((2-methoxy-4- |
| | (methylsulfonyl)phenyl)amino)prop-1-yn-1-yl)-3-(thiazol-4-yl)benzo[b]thiophene 1,1-dioxide; |
| 1158 | 7-((4-(dimethylamino)cyclohexyl)amino)-2-(3-((2-methoxy-4-(methylsulfonyl)phenyl)amino)prop-1-yn-1- |
| | yl)-3-(thiazol-4-yl)benzo[b]thiophene 1,1-dioxide; |
| 1159 | 7-((2-(dimethylamino)ethyl)amino)-2-(3-((2-methoxy-4-(methylsulfonyl)phenyl)amino)prop-1-yn-1-yl)-3- |
| | (thiazol-4-yl)benzo[b]thiophene 1,1-dioxide; |
| 1160 | 7-(((3S,4R)-3-fluoro-1-methylpiperidin-4-yl)amino)-2-(3-((2-methoxy-4- |
| | (methylsulfonyl)phenyl)amino)prop-1-yn-1-yl)-3-(thiazol-4-yl)benzo[b]thiophene 1,1-dioxide; |
| 1161 | 2-(3-((2-methoxy-4-(methylsulfonyl)phenyl)amino)prop-1-yn-1-yl)-7-((1-methylpiperidin-4-yl)amino)-3- |
| | (thiazol-4-yl)benzo[b]thiophene 1,1-dioxide; |
| 1162 | 2-(3-((2-methoxy-4-(methylsulfonyl)phenyl)amino)prop-1-yn-1-yl)-7-((tetrahydro-2H-pyran-4-yl)amino)- |
| | 3-(thiazol-4-yl)benzo[b]thiophene 1,1-dioxide; |
| 1163 | 7-((3,3-difluoro-1-methylpiperidin-4-yl)amino)-2-(3-((2-methoxy-4-(methylsulfonyl)phenyl)amino)prop-1- |
| | yn-1-yl)-3-(2,2,2-trifluoroethyl)benzo[b]thiophene 1,1-dioxide; |
| 1164 | 7-((1,1-dioxidotetrahydro-2H-thiopyran-4-yl)amino)-2-(3-((2-methoxy-4- |
| | (methylsulfonyl)phenyl)amino)prop-1-yn-1-yl)-3-(2,2,2-trifluoroethyl)benzo[b]thiophene 1,1-dioxide; |
| 1165 | 7-(((3R,4S)-3-fluoro-1-methylpiperidin-4-yl)amino)-2-(3-((2-methoxy-4- |
| | (methylsulfonyl)phenyl)amino)prop-1-yn-1-yl)-3-(2,2,2-trifluoroethyl)benzo[b]thiophene 1,1-dioxide; |
| 1166 | 7-((3-fluoro-1-methylpiperidin-4-yl)amino)-2-(3-((2-methoxy-4-(methylsulfonyl)phenyl)amino)prop-1-yn- |
| | 1-yl)-3-(2,2,2-trifluoroethyl)benzo[b]thiophene 1,1-dioxide; |
| 1167 | 2-(dimethylamino)-1-(4-((2-(3-((2-methoxy-4-(methylsulfonyl)phenyl)amino)prop-1-yn-1-yl)-1,1-dioxido- |
| | 3-(2,2,2-trifluoroethyl)benzo[b]thiophen-7-yl)amino)piperidin-1-yl)ethan-1-one; |
| 1168 | 7-((3-fluoro-1-(2-hydroxy-3-methoxypropyl)piperidin-4-yl)amino)-2-(3-((2-methoxy-4- |
| | (methylsulfonyl)phenyl)amino)prop-1-yn-1-yl)-3-(2,2,2-trifluoroethyl)benzo[b]thiophene 1,1-dioxide; |
| 1169 | 7-(((3R,4R)-3-fluoro-1-methylpiperidin-4-yl)amino)-2-(3-((2-methoxy-4- |
| | (methylsulfonyl)phenyl)amino)prop-1-yn-1-yl)-3-(2,2,2-trifluoroethyl)benzo[b]thiophene 1,1-dioxide; |
| 1170 | 7-(((3S,4S)-3-fluoro-1-methylpiperidin-4-yl)amino)-2-(3-((2-methoxy-4- |
| | (methylsulfonyl)phenyl)amino)prop-1-yn-1-yl)-3-(2,2,2-trifluoroethyl)benzo[b]thiophene 1,1-dioxide; |
| 1171 | 7-((4-(dimethylamino)cyclohexyl)amino)-2-(3-((2-methoxy-4-(methylsulfonyl)phenyl)amino)prop-1-yn-1- |
| | yl)-3-(2,2,2-trifluoroethyl)benzo[b]thiophene 1,1-dioxide; |
| 1172 | 7-((2-(dimethylamino)ethyl)amino)-2-(3-((2-methoxy-4-(methylsulfonyl)phenyl)amino)prop-1-yn-1-yl)-3- |
| | (2,2,2-trifluoroethyl)benzo[b]thiophene 1,1-dioxide; |
| 1173 | 7-(((3S,4R)-3-fluoro-1-methylpiperidin-4-yl)amino)-2-(3-((2-methoxy-4- |
| | (methylsulfonyl)phenyl)amino)prop-1-yn-1-yl)-3-(2,2,2-trifluoroethyl)benzo[b]thiophene 1,1-dioxide; |
| 1174 | 2-(3-((2-methoxy-4-(methylsulfonyl)phenyl)amino)prop-1-yn-1-yl)-7-((1-methylpiperidin-4-yl)amino)-3- |
| | (2,2,2-trifluoroethyl)benzo[b]thiophene 1,1-dioxide; |
| 1175 | 2-(3-((2-methoxy-4-(methylsulfonyl)phenyl)amino)prop-1-yn-1-yl)-7-((tetrahydro-2H-pyran-4-yl)amino)- |
| | 3-(2,2,2-trifluoroethyl)benzo[b]thiophene 1,1-dioxide; |
| 1176 | 3-(cyclopropylmethyl)-7-((3,3-difluoro-1-methylpiperidin-4-yl)amino)-2-(3-((2-methoxy-4- |
| | (methylsulfonyl)phenyl)amino)prop-1-yn-1-yl)benzo[b]thiophene 1,1-dioxide; |
| 1177 | 3-(cyclopropylmethyl)-7-((1,1-dioxidotetrahydro-2H-thiopyran-4-yl)amino)-2-(3-((2-methoxy-4- |
| | (methylsulfonyl)phenyl)amino)prop-1-yn-1-yl)benzo[b]thiophene 1,1-dioxide; |
| 1178 | 3-(cyclopropylmethyl)-7-(((3R,4S)-3-fluoro-1-methylpiperidin-4-yl)amino)-2-(3-((2-methoxy-4- |
| | (methylsulfonyl)phenyl)amino)prop-1-yn-1-yl)benzo[b]thiophene 1,1-dioxide; |
| 1179 | 3-(cyclopropylmethyl)-7-((3-fluoro-1-methylpiperidin-4-yl)amino)-2-(3-((2-methoxy-4- |
| | (methylsulfonyl)phenyl)amino)prop-1-yn-1-yl)benzo[b]thiophene 1,1-dioxide; |
| 1180 | 1-(4-((3-(cyclopropylmethyl)-2-(3-((2-methoxy-4-(methylsulfonyl)phenyl)amino)prop-1-yn-1-yl)-1,1- |
| | dioxidobenzo[b]thiophen-7-yl)amino)piperidin-1-yl)-2-(dimethylamino)ethan-1-one; |
| 1181 | 3-(cyclopropylmethyl)-7-((3-fluoro-1-(2-hydroxy-3-methoxypropyl)piperidin-4-yl)amino)-2-(3-((2- |
| | methoxy-4-(methylsulfonyl)phenyl)amino)prop-1-yn-1-yl)benzo[b]thiophene 1,1-dioxide; |
| 1182 | 3-(cyclopropylmethyl)-7-(((3R,4R)-3-fluoro-1-methylpiperidin-4-yl)amino)-2-(3-((2-methoxy-4- |
| | (methylsulfonyl)phenyl)amino)prop-1-yn-1-yl)benzo[b]thiophene 1,1-dioxide; |
| 1183 | 3-(cyclopropylmethyl)-7-(((3S,4S)-3-fluoro-1-methylpiperidin-4-yl)amino)-2-(3-((2-methoxy-4- |
| | (methylsulfonyl)phenyl)amino)prop-1-yn-1-yl)benzo[b]thiophene 1,1-dioxide; |
| 1184 | 3-(cyclopropylmethyl)-7-((4-(dimethylamino)cyclohexyl)amino)-2-(3-((2-methoxy-4- |
| | (methylsulfonyl)phenyl)amino)prop-1-yn-1-yl)benzo[b]thiophene 1,1-dioxide; |
| 1185 | 3-(cyclopropylmethyl)-7-((2-(dimethylamino)ethyl)amino)-2-(3-((2-methoxy-4- |
| | (methylsulfonyl)phenyl)amino)prop-1-yn-1-yl)benzo[b]thiophene 1,1-dioxide; |
| 1186 | 3-(cyclopropylmethyl)-7-(((3S,4R)-3-fluoro-1-methylpiperidin-4-yl)amino)-2-(3-((2-methoxy-4- |
| | (methylsulfonyl)phenyl)amino)prop-1-yn-1-yl)benzo[b]thiophene 1,1-dioxide; |
| 1187 | 3-(cyclopropylmethyl)-2-(3-((2-methoxy-4-(methylsulfonyl)phenyl)amino)prop-1-yn-1-yl)-7-((1- |
| | methylpiperidin-4-yl)amino)benzo[b]thiophene 1,1-dioxide; |
| 1188 | 3-(cyclopropylmethyl)-2-(3-((2-methoxy-4-(methylsulfonyl)phenyl)amino)prop-1-yn-1-yl)-7-((tetrahydro- |
| | 2H-pyran-4-yl)amino)benzo[b]thiophene 1,1-dioxide; |
| 1189 | 7-((3,3-difluoro-1-methylpiperidin-4-yl)amino)-3-(2,2-difluoroethyl)-2-(3-((2-methoxy-4- |
| | (methylsulfonyl)phenyl)amino)prop-1-yn-1-yl)benzo[b]thiophene 1,1-dioxide; |
| 1190 | 3-(2,2-difluoroethyl)-7-((1,1-dioxidotetrahydro-2H-thiopyran-4-yl)amino)-2-(3-((2-methoxy-4- |
| | (methylsulfonyl)phenyl)amino)prop-1-yn-1-yl)benzo[b]thiophene 1,1-dioxide; |
| 1191 | 3-(2,2-difluoroethyl)-7-(((3R,4S)-3-fluoro-1-methylpiperidin-4-yl)amino)-2-(3-((2-methoxy-4- |
| | (methylsulfonyl)phenyl)amino)prop-1-yn-1-yl)benzo[b]thiophene 1,1-dioxide; |
| 1192 | 3-(2,2-difluoroethyl)-7-((3-fluoro-1-methylpiperidin-4-yl)amino)-2-(3-((2-methoxy-4- |
| | (methylsulfonyl)phenyl)amino)prop-1-yn-1-yl)benzo[b]thiophene 1,1-dioxide; |
| 1193 | 1-(4-((3-(2,2-difluoroethyl)-2-(3-((2-methoxy-4-(methylsulfonyl)phenyl)amino)prop-1-yn-1-yl)-1,1- |
| | dioxidobenzo[b]thiophen-7-yl)amino)piperidin-1-yl)-2-(dimethylamino)ethan-1-one; |
| 1194 | 3-(2,2-difluoroethyl)-7-((3-fluoro-1-(2-hydroxy-3-methoxypropyl)piperidin-4-yl)amino)-2-(3-((2-methoxy- |
| | 4-(methylsulfonyl)phenyl)amino)prop-1-yn-1-yl)benzo[b]thiophene 1,1-dioxide; |
| 1195 | 3-(2,2-difluoroethyl)-7-(((3R,4R)-3-fluoro-1-methylpiperidin-4-yl)amino)-2-(3-((2-methoxy-4- |
| | (methylsulfonyl)phenyl)amino)prop-1-yn-1-yl)benzo[b]thiophene 1,1-dioxide; |
| 1196 | 3-(2,2-difluoroethyl)-7-(((3S,4S)-3-fluoro-1-methylpiperidin-4-yl)amino)-2-(3-((2-methoxy-4- |
| | (methylsulfonyl)phenyl)amino)prop-1-yn-1-yl)benzo[b]thiophene 1,1-dioxide; |
| 1197 | 3-(2,2-difluoroethyl)-7-((4-(dimethylamino)cyclohexyl)amino)-2-(3-((2-methoxy-4- |
| | (methylsulfonyl)phenyl)amino)prop-1-yn-1-yl)benzo[b]thiophene 1,1-dioxide; |
| 1198 | 3-(2,2-difluoroethyl)-7-((2-(dimethylamino)ethyl)amino)-2-(3-((2-methoxy-4- |
| | (methylsulfonyl)phenyl)amino)prop-1-yn-1-yl)benzo[b]thiophene 1,1-dioxide; |
| 1199 | 3-(2,2-difluoroethyl)-7-(((3S,4R)-3-fluoro-1-methylpiperidin-4-yl)amino)-2-(3-((2-methoxy-4- |
| | (methylsulfonyl)phenyl)amino)prop-1-yn-1-yl)benzo[b]thiophene 1,1-dioxide; |
| 1200 | 3-(2,2-difluoroethyl)-2-(3-((2-methoxy-4-(methylsulfonyl)phenyl)amino)prop-1-yn-1-yl)-7-((1- |
| | methylpiperidin-4-yl)amino)benzo[b]thiophene 1,1-dioxide; |
| 1201 | 3-(2,2-difluoroethyl)-2-(3-((2-methoxy-4-(methylsulfonyl)phenyl)amino)prop-1-yn-1-yl)-7-((tetrahydro- |
| | 2H-pyran-4-yl)amino)benzo[b]thiophene 1,1-dioxide; |
| 1202 | 7-((3,3-difluoro-1-methylpiperidin-4-yl)amino)-2-(3-((2-methoxy-4-(methylsulfonyl)phenyl)amino)prop-1- |
| | yn-1-yl)-3-(1H-pyrrol-1-yl)benzo[b]thiophene 1,1-dioxide; |
| 1203 | 7-((1,1-dioxidotetrahydro-2H-thiopyran-4-yl)amino)-2-(3-((2-methoxy-4- |
| | (methylsulfonyl)phenyl)amino)prop-1-yn-1-yl)-3-(1H-pyrrol-1-yl)benzo[b]thiophene 1,1-dioxide; |
| 1204 | 7-(((3R,4S)-3-fluoro-1-methylpiperidin-4-yl)amino)-2-(3-((2-methoxy-4- |
| | (methylsulfonyl)phenyl)amino)prop-1-yn-1-yl)-3-(1H-pyrrol-1-yl)benzo[b]thiophene 1,1-dioxide; |
| 1205 | 7-((3-fluoro-1-methylpiperidin-4-yl)amino)-2-(3-((2-methoxy-4-(methylsulfonyl)phenyl)amino)prop-1-yn- |
| | 1-yl)-3-(1H-pyrrol-1-yl)benzo[b]thiophene 1,1-dioxide; |
| 1206 | 2-(dimethylamino)-1-(4-((2-(3-((2-methoxy-4-(methylsulfonyl)phenyl)amino)prop-1-yn-1-yl)-1,1-dioxido- |
| | 3-(1H-pyrrol-1-yl)benzo[b]thiophen-7-yl)amino)piperidin-1-yl)ethan-1-one; |
| 1207 | 7-((3-fluoro-1-(2-hydroxy-3-methoxypropyl)piperidin-4-yl)amino)-2-(3-((2-methoxy-4- |
| | (methylsulfonyl)phenyl)amino)prop-1-yn-1-yl)-3-(1H-pyrrol-1-yl)benzo[b]thiophene 1,1-dioxide; |
| 1208 | 7-(((3R,4R)-3-fluoro-1-methylpiperidin-4-yl)amino)-2-(3-((2-methoxy-4- |
| | (methylsulfonyl)phenyl)amino)prop-1-yn-1-yl)-3-(1H-pyrrol-1-yl)benzo[b]thiophene 1,1-dioxide; |
| 1209 | 7-(((3S,4S)-3-fluoro-1-methylpiperidin-4-yl)amino)-2-(3-((2-methoxy-4- |
| | (methylsulfonyl)phenyl)amino)prop-1-yn-1-yl)-3-(1H-pyrrol-1-yl)benzo[b]thiophene 1,1-dioxide; |
| 1210 | 7-((4-(dimethylamino)cyclohexyl)amino)-2-(3-((2-methoxy-4-(methylsulfonyl)phenyl)amino)prop-1-yn-1- |
| | yl)-3-(1H-pyrrol-1-yl)benzo[b]thiophene 1,1-dioxide; |
| 1211 | 7-((2-(dimethylamino)ethyl)amino)-2-(3-((2-methoxy-4-(methylsulfonyl)phenyl)amino)prop-1-yn-1-yl)-3- |
| | (1H-pyrrol-1-yl)benzo[b]thiophene 1,1-dioxide; |
| 1212 | 7-(((3S,4R)-3-fluoro-1-methylpiperidin-4-yl)amino)-2-(3-((2-methoxy-4- |
| | (methylsulfonyl)phenyl)amino)prop-1-yn-1-yl)-3-(1H-pyrrol-1-yl)benzo[b]thiophene 1,1-dioxide; |
| 1213 | 2-(3-((2-methoxy-4-(methylsulfonyl)phenyl)amino)prop-1-yn-1-yl)-7-((1-methylpiperidin-4-yl)amino)-3- |
| | (1H-pyrrol-1-yl)benzo[b]thiophene 1,1-dioxide; |
| 1214 | 2-(3-((2-methoxy-4-(methylsulfonyl)phenyl)amino)prop-1-yn-1-yl)-3-(1H-pyrrol-1-yl)-7-((tetrahydro-2H- |
| | pyran-4-yl)amino)benzo[b]thiophene 1,1-dioxide; |
| 1215 | 7-((3,3-difluoro-1-methylpiperidin-4-yl)amino)-3-ethyl-2-(3-((2-methoxy-4- |
| | (methylsulfonyl)phenyl)amino)prop-1-yn-1-yl)benzo[b]thiophene 1,1-dioxide; |
| 1216 | 7-((1,1-dioxidotetrahydro-2H-thiopyran-4-yl)amino)-3-ethyl-2-(3-((2-methoxy-4- |
| | (methylsulfonyl)phenyl)amino)prop-1-yn-1-yl)benzo[b]thiophene 1,1-dioxide; |
| 1217 | 3-ethyl-7-(((3R,4S)-3-fluoro-1-methylpiperidin-4-yl)amino)-2-(3-((2-methoxy-4- |
| | (methylsulfonyl)phenyl)amino)prop-1-yn-1-yl)benzo[b]thiophene 1,1-dioxide; |
| 1218 | 3-ethyl-7-((3-fluoro-1-methylpiperidin-4-yl)amino)-2-(3-((2-methoxy-4- |
| | (methylsulfonyl)phenyl)amino)prop-1-yn-1-yl)benzo[b]thiophene 1,1-dioxide; |
| 1219 | 2-(dimethylamino)-1-(4-((3-ethyl-2-(3-((2-methoxy-4-(methylsulfonyl)phenyl)amino)prop-1-yn-1-yl)-1,1- |
| | dioxidobenzo[b]thiophen-7-yl)amino)piperidin-1-yl)ethan-1-one; |
| 1220 | 3-ethyl-7-((3-fluoro-1-(2-hydroxy-3-methoxypropyl)piperidin-4-yl)amino)-2-(3-((2-methoxy-4- |
| | (methylsulfonyl)phenyl)amino)prop-1-yn-1-yl)benzo[b]thiophene 1,1-dioxide; |
| 1221 | 3-ethyl-7-(((3R,4R)-3-fluoro-1-methylpiperidin-4-yl)amino)-2-(3-((2-methoxy-4- |
| | (methylsulfonyl)phenyl)amino)prop-1-yn-1-yl)benzo[b]thiophene 1,1-dioxide; |
| 1222 | 3-ethyl-7-(((3S,4S)-3-fluoro-1-methylpiperidin-4-yl)amino)-2-(3-((2-methoxy-4- |
| | (methylsulfonyl)phenyl)amino)prop-1-yn-1-yl)benzo[b]thiophene 1,1-dioxide; |
| 1223 | 7-((4-(dimethylamino)cyclohexyl)amino)-3-ethyl-2-(3-((2-methoxy-4-(methylsulfonyl)phenyl)amino)prop- |
| | 1-yn-1-yl)benzo[b]thiophene 1,1-dioxide; |
| 1224 | 7-((2-(dimethylamino)ethyl)amino)-3-ethyl-2-(3-((2-methoxy-4-(methylsulfonyl)phenyl)amino)prop-1-yn- |
| | 1-yl)benzo[b]thiophene 1,1-dioxide; |
| 1225 | 3-ethyl-7-(((3S,4R)-3-fluoro-1-methylpiperidin-4-yl)amino)-2-(3-((2-methoxy-4- |
| | (methylsulfonyl)phenyl)amino)prop-1-yn-1-yl)benzo[b]thiophene 1,1-dioxide; |
| 1226 | 3-ethyl-2-(3-((2-methoxy-4-(methylsulfonyl)phenyl)amino)prop-1-yn-1-yl)-7-((1-methylpiperidin-4- |
| | yl)amino)benzo[b]thiophene 1,1-dioxide; |
| 1227 | 3-ethyl-2-(3-((2-methoxy-4-(methylsulfonyl)phenyl)amino)prop-1-yn-1-yl)-7-((tetrahydro-2H-pyran-4- |
| | yl)amino)benzo[b]thiophene 1,1-dioxide; |
| 1228 | 4-((3-(3-(cyanomethyl)-7-((3,3-difluoro-1-methylpiperidin-4-yl)amino)-1-oxidobenzo[b]thiophen-2- |
| | yl)prop-2-yn-1-yl)amino)-3-methoxybenzenesulfonamide; |
| 1229 | 4-((3-(3-(cyanomethyl)-7-((1,1-dioxidotetrahydro-2H-thiopyran-4-yl)amino)-1-oxidobenzo[b]thiophen-2- |
| | yl)prop-2-yn-1-yl)amino)-3-methoxybenzenesulfonamide; |
| 1230 | 4-((3-(3-(cyanomethyl)-7-(((3R,4S)-3-fluoro-1-methylpiperidin-4-yl)amino)-1-oxidobenzo[b]thiophen-2- |
| | yl)prop-2-yn-1-yl)amino)-3-methoxybenzenesulfonamide; |
| 1231 | 4-((3-(3-(cyanomethyl)-7-((3-fluoro-1-methylpiperidin-4-yl)amino)-1-oxidobenzo[b]thiophen-2-yl)prop-2- |
| | yn-1-yl)amino)-3-methoxybenzenesulfonamide; |
| 1232 | 4-((3-(3-(cyanomethyl)-7-((1-(dimethylglycyl)piperidin-4-yl)amino)-1-oxidobenzo[b]thiophen-2-yl)prop-2- |
| | yn-1-yl)amino)-3-methoxybenzenesulfonamide; |
| 1233 | 4-((3-(3-(cyanomethyl)-7-((3-fluoro-1-(2-hydroxy-3-methoxypropyl)piperidin-4-yl)amino)-1- |
| | oxidobenzo[b]thiophen-2-yl)prop-2-yn-1-yl)amino)-3-methoxybenzenesulfonamide; |
| 1234 | 4-((3-(3-(cyanomethyl)-7-(((3R,4R)-3-fluoro-1-methylpiperidin-4-yl)amino)-1-oxidobenzo[b]thiophen-2- |
| | yl)prop-2-yn-1-yl)amino)-3-methoxybenzenesulfonamide; |
| 1235 | 4-((3-(3-(cyanomethyl)-7-(((3S,4S)-3-fluoro-1-methylpiperidin-4-yl)amino)-1-oxidobenzo[b]thiophen-2- |
| | yl)prop-2-yn-1-yl)amino)-3-methoxybenzenesulfonamide; |
| 1236 | 4-((3-(3-(cyanomethyl)-7-((4-(dimethylamino)cyclohexyl)amino)-1-oxidobenzo[b]thiophen-2-yl)prop-2-yn- |
| | 1-yl)amino)-3-methoxybenzenesulfonamide; |
| 1237 | 4-((3-(3-(cyanomethyl)-7-((2-(dimethylamino)ethyl)amino)-1-oxidobenzo[b]thiophen-2-yl)prop-2-yn-1- |
| | yl)amino)-3-methoxybenzenesulfonamide; |
| 1238 | 4-((3-(3-(cyanomethyl)-7-(((3S,4R)-3-fluoro-1-methylpiperidin-4-yl)amino)-1-oxidobenzo[b]thiophen-2- |
| | yl)prop-2-yn-1-yl)amino)-3-methoxybenzenesulfonamide; |
| 1239 | 4-((3-(3-(cyanomethyl)-7-((1-methylpiperidin-4-yl)amino)-1-oxidobenzo[b]thiophen-2-yl)prop-2-yn-1- |
| | yl)amino)-3-methoxybenzenesulfonamide; |
| 1240 | 4-((3-(3-(cyanomethyl)-1-oxido-7-((tetrahydro-2H-pyran-4-yl)amino)benzo[b]thiophen-2-yl)prop-2-yn-1- |
| | yl)amino)-3-methoxybenzenesulfonamide; |
| 1241 | 4-((3-(7-((3,3-difluoro-1-methylpiperidin-4-yl)amino)-1-oxido-3-(thiazol-4-yl)benzo[b]thiophen-2-yl)prop- |
| | 2-yn-1-yl)amino)-3-methoxybenzenesulfonamide; |
| 1242 | 4-((3-(7-((1,1-dioxidotetrahydro-2H-thiopyran-4-yl)amino)-1-oxido-3-(thiazol-4-yl)benzo[b]thiophen-2- |
| | yl)prop-2-yn-1-yl)amino)-3-methoxybenzenesulfonamide; |
| 1243 | 4-((3-(7-(((3R,4S)-3-fluoro-1-methylpiperidin-4-yl)amino)-1-oxido-3-(thiazol-4-yl)benzo[b]thiophen-2- |
| | yl)prop-2-yn-1-yl)amino)-3-methoxybenzenesulfonamide; |
| 1244 | 4-((3-(7-((3-fluoro-1-methylpiperidin-4-yl)amino)-1-oxido-3-(thiazol-4-yl)benzo[b]thiophen-2-yl)prop-2- |
| | yn-1-yl)amino)-3-methoxybenzenesulfonamide; |
| 1245 | 4-((3-(7-((1-(dimethylglycyl)piperidin-4-yl)amino)-1-oxido-3-(thiazol-4-yl)benzo[b]thiophen-2-yl)prop-2- |
| | yn-1-yl)amino)-3-methoxybenzenesulfonamide; |
| 1246 | 4-((3-(7-((3-fluoro-1-(2-hydroxy-3-methoxypropyl)piperidin-4-yl)amino)-1-oxido-3-(thiazol-4- |
| | yl)benzo[b]thiophen-2-yl)prop-2-yn-1-yl)amino)-3-methoxybenzenesulfonamide; |
| 1247 | 4-((3-(7-(((3R,4R)-3-fluoro-1-methylpiperidin-4-yl)amino)-1-oxido-3-(thiazol-4-yl)benzo[b]thiophen-2- |
| | yl)prop-2-yn-1-yl)amino)-3-methoxybenzenesulfonamide; |
| 1248 | 4-((3-(7-(((3S,4S)-3-fluoro-1-methylpiperidin-4-yl)amino)-1-oxido-3-(thiazol-4-yl)benzo[b]thiophen-2- |
| | yl)prop-2-yn-1-yl)amino)-3-methoxybenzenesulfonamide; |
| 1249 | 4-((3-(7-((4-(dimethylamino)cyclohexyl)amino)-1-oxido-3-(thiazol-4-yl)benzo[b]thiophen-2-yl)prop-2-yn- |
| | 1-yl)amino)-3-methoxybenzenesulfonamide; |
| 1250 | 4-((3-(7-((2-(dimethylamino)ethyl)amino)-1-oxido-3-(thiazol-4-yl)benzo[b]thiophen-2-yl)prop-2-yn-1- |
| | yl)amino)-3-methoxybenzenesulfonamide; |
| 1251 | 4-((3-(7-(((3S,4R)-3-fluoro-1-methylpiperidin-4-yl)amino)-1-oxido-3-(thiazol-4-yl)benzo[b]thiophen-2- |
| | yl)prop-2-yn-1-yl)amino)-3-methoxybenzenesulfonamide; |
| 1252 | 3-methoxy-4-((3-(7-((1-methylpiperidin-4-yl)amino)-1-oxido-3-(thiazol-4-yl)benzo[b]thiophen-2-yl)prop- |
| | 2-yn-1-yl)amino)benzenesulfonamide; |
| 1253 | 3-methoxy-4-((3-(1-oxido-7-((tetrahydro-2H-pyran-4-yl)amino)-3-(thiazol-4-yl)benzo[b]thiophen-2- |
| | yl)prop-2-yn-1-yl)amino)benzenesulfonamide; |
| 1254 | 4-((3-(7-((3,3-difluoro-1-methylpiperidin-4-yl)amino)-1-oxido-3-(2,2,2-trifluoroethyl)benzo[b]thiophen-2- |
| | yl)prop-2-yn-1-yl)amino)-3-methoxybenzenesulfonamide; |
| 1255 | 4-((3-(7-((1,1-dioxidotetrahydro-2H-thiopyran-4-yl)amino)-1-oxido-3-(2,2,2- |
| | trifluoroethyl)benzo[b]thiophen-2-yl)prop-2-yn-1-yl)amino)-3-methoxybenzenesulfonamide; |
| 1256 | 4-((3-(7-(((3R,4S)-3-fluoro-1-methylpiperidin-4-yl)amino)-1-oxido-3-(2,2,2- |
| | trifluoroethyl)benzo[b]thiophen-2-yl)prop-2-yn-1-yl)amino)-3-methoxybenzenesulfonamide; |
| 1257 | 4-((3-(7-((3-fluoro-1-methylpiperidin-4-yl)amino)-1-oxido-3-(2,2,2-trifluoroethyl)benzo[b]thiophen-2- |
| | yl)prop-2-yn-1-yl)amino)-3-methoxybenzenesulfonamide; |
| 1258 | 4-((3-(7-((1-(dimethylglycyl)piperidin-4-yl)amino)-1-oxido-3-(2,2,2-trifluoroethyl)benzo[b]thiophen-2- |
| | yl)prop-2-yn-1-yl)amino)-3-methoxybenzenesulfonamide; |
| 1259 | 4-((3-(7-((3-fluoro-1-(2-hydroxy-3-methoxypropyl)piperidin-4-yl)amino)-1-oxido-3-(2,2,2- |
| | trifluoroethyl)benzo[b]thiophen-2-yl)prop-2-yn-1-yl)amino)-3-methoxybenzenesulfonamide; |
| 1260 | 4-((3-(7-(((3R,4R)-3-fluoro-1-methylpiperidin-4-yl)amino)-1-oxido-3-(2,2,2- |
| | trifluoroethyl)benzo[b]thiophen-2-yl)prop-2-yn-1-yl)amino)-3-methoxybenzenesulfonamide; |
| 1261 | 4-((3-(7-(((3S,4S)-3-fluoro-1-methylpiperidin-4-yl)amino)-1-oxido-3-(2,2,2- |
| | trifluoroethyl)benzo[b]thiophen-2-yl)prop-2-yn-1-yl)amino)-3-methoxybenzenesulfonamide; |
| 1262 | 4-((3-(7-((4-(dimethylamino)cyclohexyl)amino)-1-oxido-3-(2,2,2-trifluoroethyl)benzo[b]thiophen-2- |
| | yl)prop-2-yn-1-yl)amino)-3-methoxybenzenesulfonamide; |
| 1263 | 4-((3-(7-((2-(dimethylamino)ethyl)amino)-1-oxido-3-(2,2,2-trifluoroethyl)benzo[b]thiophen-2-yl)prop-2- |
| | yn-1-yl)amino)-3-methoxybenzenesulfonamide; |
| 1264 | 4-((3-(7-(((3S,4R)-3-fluoro-1-methylpiperidin-4-yl)amino)-1-oxido-3-(2,2,2- |
| | trifluoroethyl)benzo[b]thiophen-2-yl)prop-2-yn-1-yl)amino)-3-methoxybenzenesulfonamide; |
| 1265 | 3-methoxy-4-((3-(7-((1-methylpiperidin-4-yl)amino)-1-oxido-3-(2,2,2-trifluoroethyl)benzo[b]thiophen-2- |
| | yl)prop-2-yn-1-yl)amino)benzenesulfonamide; |
| 1266 | 3-methoxy-4-((3-(1-oxido-7-((tetrahydro-2H-pyran-4-yl)amino)-3-(2,2,2-trifluoroethyl)benzo[b]thiophen-2- |
| | yl)prop-2-yn-1-yl)amino)benzenesulfonamide; |
| 1267 | 4-((3-(3-(cyclopropylmethyl)-7-((3,3-difluoro-1-methylpiperidin-4-yl)amino)-1-oxidobenzo[b]thiophen-2- |
| | yl)prop-2-yn-1-yl)amino)-3-methoxybenzenesulfonamide; |
| 1268 | 4-((3-(3-(cyclopropylmethyl)-7-((1,1-dioxidotetrahydro-2H-thiopyran-4-yl)amino)-1- |
| | oxidobenzo[b]thiophen-2-yl)prop-2-yn-1-yl)amino)-3-methoxybenzenesulfonamide; |
| 1269 | 4-((3-(3-(cyclopropylmethyl)-7-(((3R,4S)-3-fluoro-1-methylpiperidin-4-yl)amino)-1- |
| | oxidobenzo[b]thiophen-2-yl)prop-2-yn-1-yl)amino)-3-methoxybenzenesulfonamide; |
| 1270 | 4-((3-(3-(cyclopropylmethyl)-7-((3-fluoro-1-methylpiperidin-4-yl)amino)-1-oxidobenzo[b]thiophen-2- |
| | yl)prop-2-yn-1-yl)amino)-3-methoxybenzenesulfonamide; |
| 1271 | 4-((3-(3-(cyclopropylmethyl)-7-((1-(dimethylglycyl)piperidin-4-yl)amino)-1-oxidobenzo[b]thiophen-2- |
| | yl)prop-2-yn-1-yl)amino)-3-methoxybenzenesulfonamide; |
| 1272 | 4-((3-(3-(cyclopropylmethyl)-7-((3-fluoro-1-(2-hydroxy-3-methoxypropyl)piperidin-4-yl)amino)-1- |
| | oxidobenzo[b]thiophen-2-yl)prop-2-yn-1-yl)amino)-3-methoxybenzenesulfonamide; |
| 1273 | 4-((3-(3-(cyclopropylmethyl)-7-(((3R,4R)-3-fluoro-1-methylpiperidin-4-yl)amino)-1- |
| | oxidobenzo[b]thiophen-2-yl)prop-2-yn-1-yl)amino)-3-methoxybenzenesulfonamide; |
| 1274 | 4-((3-(3-(cyclopropylmethyl)-7-(((3S,4S)-3-fluoro-1-methylpiperidin-4-yl)amino)-1- |
| | oxidobenzo[b]thiophen-2-yl)prop-2-yn-1-yl)amino)-3-methoxybenzenesulfonamide; |
| 1275 | 4-((3-(3-(cyclopropylmethyl)-7-((4-(dimethylamino)cyclohexyl)amino)-1-oxidobenzo[b]thiophen-2- |
| | yl)prop-2-yn-1-yl)amino)-3-methoxybenzenesulfonamide; |
| 1276 | 4-((3-(3-(cyclopropylmethyl)-7-((2-(dimethylamino)ethyl)amino)-1-oxidobenzo[b]thiophen-2-yl)prop-2-yn- |
| | 1-yl)amino)-3-methoxybenzenesulfonamide; |
| 1277 | 4-((3-(3-(cyclopropylmethyl)-7-(((3S,4R)-3-fluoro-1-methylpiperidin-4-yl)amino)-1- |
| | oxidobenzo[b]thiophen-2-yl)prop-2-yn-1-yl)amino)-3-methoxybenzenesulfonamide; |
| 1278 | 4-((3-(3-(cyclopropylmethyl)-7-((1-methylpiperidin-4-yl)amino)-1-oxidobenzo[b]thiophen-2-yl)prop-2-yn- |
| | 1-yl)amino)-3-methoxybenzenesulfonamide; |
| 1279 | 4-((3-(3-(cyclopropylmethyl)-1-oxido-7-((tetrahydro-2H-pyran-4-yl)amino)benzo[b]thiophen-2-yl)prop-2- |
| | yn-1-yl)amino)-3-methoxybenzenesulfonamide; |
| 1280 | 4-((3-(7-((3,3-difluoro-1-methylpiperidin-4-yl)amino)-3-(2,2-difluoroethyl)-1-oxidobenzo[b]thiophen-2- |
| | yl)prop-2-yn-1-yl)amino)-3-methoxybenzenesulfonamide; |
| 1281 | 4-((3-(3-(2,2-difluoroethyl)-7-((1,1-dioxidotetrahydro-2H-thiopyran-4-yl)amino)-1-oxidobenzo[b]thiophen- |
| | 2-yl)prop-2-yn-1-yl)amino)-3-methoxybenzenesulfonamide; |
| 1282 | 4-((3-(3-(2,2-difluoroethyl)-7-(((3R,4S)-3-fluoro-1-methylpiperidin-4-yl)amino)-1-oxidobenzo[b]thiophen- |
| | 2-yl)prop-2-yn-1-yl)amino)-3-methoxybenzenesulfonamide; |
| 1283 | 4-((3-(3-(2,2-difluoroethyl)-7-((3-fluoro-1-methylpiperidin-4-yl)amino)-1-oxidobenzo[b]thiophen-2- |
| | yl)prop-2-yn-1-yl)amino)-3-methoxybenzenesulfonamide; |
| 1284 | 4-((3-(3-(2,2-difluoroethyl)-7-((1-(dimethylglycyl)piperidin-4-yl)amino)-1-oxidobenzo[b]thiophen-2- |
| | yl)prop-2-yn-1-yl)amino)-3-methoxybenzenesulfonamide; |
| 1285 | 4-((3-(3-(2,2-difluoroethyl)-7-((3-fluoro-1-(2-hydroxy-3-methoxypropyl)piperidin-4-yl)amino)-1- |
| | oxidobenzo[b]thiophen-2-yl)prop-2-yn-1-yl)amino)-3-methoxybenzenesulfonamide; |
| 1286 | 4-((3-(3-(2,2-difluoroethyl)-7-(((3R,4R)-3-fluoro-1-methylpiperidin-4-yl)amino)-1-oxidobenzo[b]thiophen- |
| | 2-yl)prop-2-yn-1-yl)amino)-3-methoxybenzenesulfonamide; |
| 1287 | 4-((3-(3-(2,2-difluoroethyl)-7-(((3S,4S)-3-fluoro-1-methylpiperidin-4-yl)amino)-1-oxidobenzo[b]thiophen- |
| | 2-yl)prop-2-yn-1-yl)amino)-3-methoxybenzenesulfonamide; |
| 1288 | 4-((3-(3-(2,2-difluoroethyl)-7-((4-(dimethylamino)cyclohexyl)amino)-1-oxidobenzo[b]thiophen-2-yl)prop- |
| | 2-yn-1-yl)amino)-3-methoxybenzenesulfonamide; |
| 1289 | 4-((3-(3-(2,2-difluoroethyl)-7-((2-(dimethylamino)ethyl)amino)-1-oxidobenzo[b]thiophen-2-yl)prop-2-yn- |
| | 1-yl)amino)-3-methoxybenzenesulfonamide; |
| 1290 | 4-((3-(3-(2,2-difluoroethyl)-7-(((3S,4R)-3-fluoro-1-methylpiperidin-4-yl)amino)-1-oxidobenzo[b]thiophen- |
| | 2-yl)prop-2-yn-1-yl)amino)-3-methoxybenzenesulfonamide; |
| 1291 | 4-((3-(3-(2,2-difluoroethyl)-7-((1-methylpiperidin-4-yl)amino)-1-oxidobenzo[b]thiophen-2-yl)prop-2-yn-1- |
| | yl)amino)-3-methoxybenzenesulfonamide; |
| 1292 | 4-((3-(3-(2,2-difluoroethyl)-1-oxido-7-((tetrahydro-2H-pyran-4-yl)amino)benzo[b]thiophen-2-yl)prop-2-yn- |
| | 1-yl)amino)-3-methoxybenzenesulfonamide; |
| 1293 | 4-((3-(7-((3,3-difluoro-1-methylpiperidin-4-yl)amino)-1-oxido-3-(1H-pyrrol-1-yl)benzo[b]thiophen-2- |
| | yl)prop-2-yn-1-yl)amino)-3-methoxybenzenesulfonamide; |
| 1294 | 4-((3-(7-((1,1-dioxidotetrahydro-2H-thiopyran-4-yl)amino)-1-oxido-3-(1H-pyrrol-1-yl)benzo[b]thiophen-2- |
| | yl)prop-2-yn-1-yl)amino)-3-methoxybenzenesulfonamide; |
| 1295 | 4-((3-(7-(((3R,4S)-3-fluoro-1-methylpiperidin-4-yl)amino)-1-oxido-3-(1H-pyrrol-1-yl)benzo[b]thiophen-2- |
| | yl)prop-2-yn-1-yl)amino)-3-methoxybenzenesulfonamide; |
| 1296 | 4-((3-(7-((3-fluoro-1-methylpiperidin-4-yl)amino)-1-oxido-3-(1H-pyrrol-1-yl)benzo[b]thiophen-2-yl)prop- |
| | 2-yn-1-yl)amino)-3-methoxybenzenesulfonamide; |
| 1297 | 4-((3-(7-((1-(dimethylglycyl)piperidin-4-yl)amino)-1-oxido-3-(1H-pyrrol-1-yl)benzo[b]thiophen-2-yl)prop- |
| | 2-yn-1-yl)amino)-3-methoxybenzenesulfonamide; |
| 1298 | 4-((3-(7-((3-fluoro-1-(2-hydroxy-3-methoxypropyl)piperidin-4-yl)amino)-1-oxido-3-(1H-pyrrol-1- |
| | yl)benzo[b]thiophen-2-yl)prop-2-yn-1-yl)amino)-3-methoxybenzenesulfonamide; |
| 1299 | 4-((3-(7-(((3R,4R)-3-fluoro-1-methylpiperidin-4-yl)amino)-1-oxido-3-(1H-pyrrol-1-yl)benzo[b]thiophen-2- |
| | yl)prop-2-yn-1-yl)amino)-3-methoxybenzenesulfonamide; |
| 1300 | 4-((3-(7-(((3S,4S)-3-fluoro-1-methylpiperidin-4-yl)amino)-1-oxido-3-(1H-pyrrol-1-yl)benzo[b]thiophen-2- |
| | yl)prop-2-yn-1-yl)amino)-3-methoxybenzenesulfonamide; |
| 1301 | 4-((3-(7-((4-(dimethylamino)cyclohexyl)amino)-1-oxido-3-(1H-pyrrol-1-yl)benzo[b]thiophen-2-yl)prop-2- |
| | yn-1-yl)amino)-3-methoxybenzenesulfonamide; |
| 1302 | 4-((3-(7-((2-(dimethylamino)ethyl)amino)-1-oxido-3-(1H-pyrrol-1-yl)benzo[b]thiophen-2-yl)prop-2-yn-1- |
| | yl)amino)-3-methoxybenzenesulfonamide; |
| 1303 | 4-((3-(7-(((3S,4R)-3-fluoro-1-methylpiperidin-4-yl)amino)-1-oxido-3-(1H-pyrrol-1-yl)benzo[b]thiophen-2- |
| | yl)prop-2-yn-1-yl)amino)-3-methoxybenzenesulfonamide; |
| 1304 | 3-methoxy-4-((3-(7-((1-methylpiperidin-4-yl)amino)-1-oxido-3-(1H-pyrrol-1-yl)benzo[b]thiophen-2- |
| | yl)prop-2-yn-1-yl)amino)benzenesulfonamide; |
| 1305 | 3-methoxy-4-((3-(1-oxido-3-(1H-pyrrol-1-yl)-7-((tetrahydro-2H-pyran-4-yl)amino)benzo[b]thiophen-2- |
| | yl)prop-2-yn-1-yl)amino)benzenesulfonamide; |
| 1306 | 4-((3-(7-((3,3-difluoro-1-methylpiperidin-4-yl)amino)-3-ethyl-1-oxidobenzo[b]thiophen-2-yl)prop-2-yn-1- |
| | yl)amino)-3-methoxybenzenesulfonamide; |
| 1307 | 4-((3-(7-((1,1-dioxidotetrahydro-2H-thiopyran-4-yl)amino)-3-ethyl-1-oxidobenzo[b]thiophen-2-yl)prop-2- |
| | yn-1-yl)amino)-3-methoxybenzenesulfonamide; |
| 1308 | 4-((3-(3-ethyl-7-(((3R,4S)-3-fluoro-1-methylpiperidin-4-yl)amino)-1-oxidobenzo[b]thiophen-2-yl)prop-2- |
| | yn-1-yl)amino)-3-methoxybenzenesulfonamide; |
| 1309 | 4-((3-(3-ethyl-7-((3-fluoro-1-methylpiperidin-4-yl)amino)-1-oxidobenzo[b]thiophen-2-yl)prop-2-yn-1- |
| | yl)amino)-3-methoxybenzenesulfonamide; |
| 1310 | 4-((3-(7-((1-(dimethylglycyl)piperidin-4-yl)amino)-3-ethyl-1-oxidobenzo[b]thiophen-2-yl)prop-2-yn-1- |
| | yl)amino)-3-methoxybenzenesulfonamide; |
| 1311 | 4-((3-(3-ethyl-7-((3-fluoro-1-(2-hydroxy-3-methoxypropyl)piperidin-4-yl)amino)-1- |
| | oxidobenzo[b]thiophen-2-yl)prop-2-yn-1-yl)amino)-3-methoxybenzenesulfonamide; |
| 1312 | 4-((3-(3-ethyl-7-(((3R,4R)-3-fluoro-1-methylpiperidin-4-yl)amino)-1-oxidobenzo[b]thiophen-2-yl)prop-2- |
| | yn-1-yl)amino)-3-methoxybenzenesulfonamide; |
| 1313 | 4-((3-(3-ethyl-7-(((3S,4S)-3-fluoro-1-methylpiperidin-4-yl)amino)-1-oxidobenzo[b]thiophen-2-yl)prop-2- |
| | yn-1-yl)amino)-3-methoxybenzenesulfonamide; |
| 1314 | 4-((3-(7-((4-(dimethylamino)cyclohexyl)amino)-3-ethyl-1-oxidobenzo[b]thiophen-2-yl)prop-2-yn-1- |
| | yl)amino)-3-methoxybenzenesulfonamide; |
| 1315 | 4-((3-(7-((2-(dimethylamino)ethyl)amino)-3-ethyl-1-oxidobenzo[b]thiophen-2-yl)prop-2-yn-1-yl)amino)-3- |
| | methoxybenzenesulfonamide; |
| 1316 | 4-((3-(3-ethyl-7-(((3S,4R)-3-fluoro-1-methylpiperidin-4-yl)amino)-1-oxidobenzo[b]thiophen-2-yl)prop-2- |
| | yn-1-yl)amino)-3-methoxybenzenesulfonamide; |
| 1317 | 4-((3-(3-ethyl-7-((1-methylpiperidin-4-yl)amino)-1-oxidobenzo[b]thiophen-2-yl)prop-2-yn-1-yl)amino)-3- |
| | methoxybenzenesulfonamide; |
| 1318 | 4-((3-(3-ethyl-1-oxido-7-((tetrahydro-2H-pyran-4-yl)amino)benzo[b]thiophen-2-yl)prop-2-yn-1-yl)amino)- |
| | 3-methoxybenzenesulfonamide; |
| 1319 | 2-(7-((3,3-difluoro-1-methylpiperidin-4-yl)amino)-2-(3-((5-fluoro-2-methoxy-4- |
| | (methylsulfonyl)phenyl)amino)prop-1-yn-1-yl)-1-oxidobenzo[b]thiophen-3-yl)acetonitrile; |
| 1320 | 2-(7-((1,1-dioxidotetrahydro-2H-thiopyran-4-yl)amino)-2-(3-((5-fluoro-2-methoxy-4- |
| | (methylsulfonyl)phenyl)amino)prop-1-yn-1-yl)-1-oxidobenzo[b]thiophen-3-yl)acetonitrile; |
| 1321 | 2-(7-(((3R,4S)-3-fluoro-1-methylpiperidin-4-yl)amino)-2-(3-((5-fluoro-2-methoxy-4- |
| | (methylsulfonyl)phenyl)amino)prop-1-yn-1-yl)-1-oxidobenzo[b]thiophen-3-yl)acetonitrile; |
| 1322 | 2-(7-((3-fluoro-1-methylpiperidin-4-yl)amino)-2-(3-((5-fluoro-2-methoxy-4- |
| | (methylsulfonyl)phenyl)amino)prop-1-yn-1-yl)-1-oxidobenzo[b]thiophen-3-yl)acetonitrile; |
| 1323 | 2-(7-((1-(dimethylglycyl)piperidin-4-yl)amino)-2-(3-((5-fluoro-2-methoxy-4- |
| | (methylsulfonyl)phenyl)amino)prop-1-yn-1-yl)-1-oxidobenzo[b]thiophen-3-yl)acetonitrile; |
| 1324 | 2-(7-((3-fluoro-1-(2-hydroxy-3-methoxypropyl)piperidin-4-yl)amino)-2-(3-((5-fluoro-2-methoxy-4- |
| | (methylsulfonyl)phenyl)amino)prop-1-yn-1-yl)-1-oxidobenzo[b]thiophen-3-yl)acetonitrile; |
| 1325 | 2-(7-(((3R,4R)-3-fluoro-1-methylpiperidin-4-yl)amino)-2-(3-((5-fluoro-2-methoxy-4- |
| | (methylsulfonyl)phenyl)amino)prop-1-yn-1-yl)-1-oxidobenzo[b]thiophen-3-yl)acetonitrile; |
| 1326 | 2-(7-(((3S,4S)-3-fluoro-1-methylpiperidin-4-yl)amino)-2-(3-((5-fluoro-2-methoxy-4- |
| | (methylsulfonyl)phenyl)amino)prop-1-yn-1-yl)-1-oxidobenzo[b]thiophen-3-yl)acetonitrile; |
| 1327 | 2-(7-((4-(dimethylamino)cyclohexyl)amino)-2-(3-((5-fluoro-2-methoxy-4- |
| | (methylsulfonyl)phenyl)amino)prop-1-yn-1-yl)-1-oxidobenzo[b]thiophen-3-yl)acetonitrile; |
| 1328 | 2-(7-((2-(dimethylamino)ethyl)amino)-2-(3-((5-fluoro-2-methoxy-4-(methylsulfonyl)phenyl)amino)prop-1- |
| | yn-1-yl)-1-oxidobenzo[b]thiophen-3-yl)acetonitrile; |
| 1329 | 2-(7-(((3S,4R)-3-fluoro-1-methylpiperidin-4-yl)amino)-2-(3-((5-fluoro-2-methoxy-4- |
| | (methylsulfonyl)phenyl)amino)prop-1-yn-1-yl)-1-oxidobenzo[b]thiophen-3-yl)acetonitrile; |
| 1330 | 2-(2-(3-((5-fluoro-2-methoxy-4-(methylsulfonyl)phenyl)amino)prop-1-yn-1-yl)-7-((1-methylpiperidin-4- |
| | yl)amino)-1-oxidobenzo[b]thiophen-3-yl)acetonitrile; |
| 1331 | 2-(2-(3-((5-fluoro-2-methoxy-4-(methylsulfonyl)phenyl)amino)prop-1-yn-1-yl)-1-oxido-7-((tetrahydro-2H- |
| | pyran-4-yl)amino)benzo[b]thiophen-3-yl)acetonitrile; |
| 1332 | 7-((3,3-difluoro-1-methylpiperidin-4-yl)amino)-2-(3-((5-fluoro-2-methoxy-4- |
| | (methylsulfonyl)phenyl)amino)prop-1-yn-1-yl)-3-(thiazol-4-yl)benzo[b]thiophene 1-oxide; |
| 1333 | 4-((2-(3-((5-fluoro-2-methoxy-4-(methylsulfonyl)phenyl)amino)prop-1-yn-1-yl)-1-oxido-3-(thiazol-4- |
| | yl)benzo[b]thiophen-7-yl)amino)tetrahydro-2H-thiopyran 1,1-dioxide; |
| 1334 | 7-(((3R,4S)-3-fluoro-1-methylpiperidin-4-yl)amino)-2-(3-((5-fluoro-2-methoxy-4- |
| | (methylsulfonyl)phenyl)amino)prop-1-yn-1-yl)-3-(thiazol-4-yl)benzo[b]thiophene 1-oxide; |
| 1335 | 7-((3-fluoro-1-methylpiperidin-4-yl)amino)-2-(3-((5-fluoro-2-methoxy-4- |
| | (methylsulfonyl)phenyl)amino)prop-1-yn-1-yl)-3-(thiazol-4-yl)benzo[b]thiophene 1-oxide; |
| 1336 | 2-(dimethylamino)-1-(4-((2-(3-((5-fluoro-2-methoxy-4-(methylsulfonyl)phenyl)amino)prop-1-yn-1-yl)-1- |
| | oxido-3-(thiazol-4-yl)benzo[b]thiophen-7-yl)amino)piperidin-1-yl)ethan-1-one; |
| 1337 | 7-((3-fluoro-1-(2-hydroxy-3-methoxypropyl)piperidin-4-yl)amino)-2-(3-((5-fluoro-2-methoxy-4- |
| | (methylsulfonyl)phenyl)amino)prop-1-yn-1-yl)-3-(thiazol-4-yl)benzo[b]thiophene 1-oxide; |
| 1338 | 7-(((3R,4R)-3-fluoro-1-methylpiperidin-4-yl)amino)-2-(3-((5-fluoro-2-methoxy-4- |
| | (methylsulfonyl)phenyl)amino)prop-1-yn-1-yl)-3-(thiazol-4-yl)benzo[b]thiophene 1-oxide; |
| 1339 | 7-(((3S,4S)-3-fluoro-1-methylpiperidin-4-yl)amino)-2-(3-((5-fluoro-2-methoxy-4- |
| | (methylsulfonyl)phenyl)amino)prop-1-yn-1-yl)-3-(thiazol-4-yl)benzo[b]thiophene 1-oxide; |
| 1340 | 7-((4-(dimethylamino)cyclohexyl)amino)-2-(3-((5-fluoro-2-methoxy-4- |
| | (methylsulfonyl)phenyl)amino)prop-1-yn-1-yl)-3-(thiazol-4-yl)benzo[b]thiophene 1-oxide; |
| 1341 | 7-((2-(dimethylamino)ethyl)amino)-2-(3-((5-fluoro-2-methoxy-4-(methylsulfonyl)phenyl)amino)prop-1-yn- |
| | 1-yl)-3-(thiazol-4-yl)benzo[b]thiophene 1-oxide; |
| 1342 | 7-(((3S,4R)-3-fluoro-1-methylpiperidin-4-yl)amino)-2-(3-((5-fluoro-2-methoxy-4- |
| | (methylsulfonyl)phenyl)amino)prop-1-yn-1-yl)-3-(thiazol-4-yl)benzo[b]thiophene 1-oxide; |
| 1343 | 2-(3-((5-fluoro-2-methoxy-4-(methylsulfonyl)phenyl)amino)prop-1-yn-1-yl)-7-((1-methylpiperidin-4- |
| | yl)amino)-3-(thiazol-4-yl)benzo[b]thiophene 1-oxide; |
| 1344 | 2-(3-((5-fluoro-2-methoxy-4-(methylsulfonyl)phenyl)amino)prop-1-yn-1-yl)-7-((tetrahydro-2H-pyran-4- |
| | yl)amino)-3-(thiazol-4-yl)benzo[b]thiophene 1-oxide; |
| 1345 | 7-((3,3-difluoro-1-methylpiperidin-4-yl)amino)-2-(3-((5-fluoro-2-methoxy-4- |
| | (methylsulfonyl)phenyl)amino)prop-1-yn-1-yl)-3-(2,2,2-trifluoroethyl)benzo[b]thiophene 1-oxide; |
| 1346 | 4-((2-(3-((5-fluoro-2-methoxy-4-(methylsulfonyl)phenyl)amino)prop-1-yn-1-yl)-1-oxido-3-(2,2,2- |
| | trifluoroethyl)benzo[b]thiophen-7-yl)amino)tetrahydro-2H-thiopyran 1,1-dioxide; |
| 1347 | 7-(((3R,4S)-3-fluoro-1-methylpiperidin-4-yl)amino)-2-(3-((5-fluoro-2-methoxy-4- |
| | (methylsulfonyl)phenyl)amino)prop-1-yn-1-yl)-3-(2,2,2-trifluoroethyl)benzo[b]thiophene 1-oxide; |
| 1348 | 7-((3-fluoro-1-methylpiperidin-4-yl)amino)-2-(3-((5-fluoro-2-methoxy-4- |
| | (methylsulfonyl)phenyl)amino)prop-1-yn-1-yl)-3-(2,2,2-trifluoroethyl)benzo[b]thiophene 1-oxide; |
| 1349 | 2-(dimethylamino)-1-(4-((2-(3-((5-fluoro-2-methoxy-4-(methylsulfonyl)phenyl)amino)prop-1-yn-1-yl)-1- |
| | oxido-3-(2,2,2-trifluoroethyl)benzo[b]thiophen-7-yl)amino)piperidin-1-yl)ethan-1-one; |
| 1350 | 7-((3-fluoro-1-(2-hydroxy-3-methoxypropyl)piperidin-4-yl)amino)-2-(3-((5-fluoro-2-methoxy-4- |
| | (methylsulfonyl)phenyl)amino)prop-1-yn-1-yl)-3-(2,2,2-trifluoroethyl)benzo[b]thiophene 1-oxide; |
| 1351 | 7-(((3R,4R)-3-fluoro-1-methylpiperidin-4-yl)amino)-2-(3-((5-fluoro-2-methoxy-4- |
| | (methylsulfonyl)phenyl)amino)prop-1-yn-1-yl)-3-(2,2,2-trifluoroethyl)benzo[b]thiophene 1-oxide; |
| 1352 | 7-(((3S,4S)-3-fluoro-1-methylpiperidin-4-yl)amino)-2-(3-((5-fluoro-2-methoxy-4- |
| | (methylsulfonyl)phenyl)amino)prop-1-yn-1-yl)-3-(2,2,2-trifluoroethyl)benzo[b]thiophene 1-oxide; |
| 1353 | 7-((4-(dimethylamino)cyclohexyl)amino)-2-(3-((5-fluoro-2-methoxy-4- |
| | (methylsulfonyl)phenyl)amino)prop-1-yn-1-yl)-3-(2,2,2-trifluoroethyl)benzo[b]thiophene 1-oxide; |
| 1354 | 7-((2-(dimethylamino)ethyl)amino)-2-(3-((5-fluoro-2-methoxy-4-(methylsulfonyl)phenyl)amino)prop-1-yn- |
| | 1-yl)-3-(2,2,2-trifluoroethyl)benzo[b]thiophene 1-oxide; |
| 1355 | 7-(((3S,4R)-3-fluoro-1-methylpiperidin-4-yl)amino)-2-(3-((5-fluoro-2-methoxy-4- |
| | (methylsulfonyl)phenyl)amino)prop-1-yn-1-yl)-3-(2,2,2-trifluoroethyl)benzo[b]thiophene 1-oxide; |
| 1356 | 2-(3-((5-fluoro-2-methoxy-4-(methylsulfonyl)phenyl)amino)prop-1-yn-1-yl)-7-((1-methylpiperidin-4- |
| | yl)amino)-3-(2,2,2-trifluoroethyl)benzo[b]thiophene 1-oxide; |
| 1357 | 2-(3-((5-fluoro-2-methoxy-4-(methylsulfonyl)phenyl)amino)prop-1-yn-1-yl)-7-((tetrahydro-2H-pyran-4- |
| | yl)amino)-3-(2,2,2-trifluoroethyl)benzo[b]thiophene 1-oxide; |
| 1358 | 3-(cyclopropylmethyl)-7-((3,3-difluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1-oxide; |
| 1359 | 4-((3-(cyclopropylmethyl)-2-(3-((5-fluoro-2-methoxy-4-(methylsulfonyl)phenyl)amino)prop-1-yn-1-yl)-1- |
| | oxidobenzo[b]thiophen-7-yl)amino)tetrahydro-2H-thiopyran 1,1-dioxide; |
| 1360 | 3-(cyclopropylmethyl)-7-(((3R,4S)-3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1-oxide; |
| 1361 | 3-(cyclopropylmethyl)-7-((3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1-oxide; |
| 1362 | 1-(4-((3-(cyclopropylmethyl)-2-(3-((5-fluoro-2-methoxy-4-(methylsulfonyl)phenyl)amino)prop-1-yn-1-yl)- |
| | 1-oxidobenzo[b]thiophen-7-yl)amino)piperidin-1-yl)-2-(dimethylamino)ethan-1-one; |
| 1363 | 3-(cyclopropylmethyl)-7-((3-fluoro-1-(2-hydroxy-3-methoxypropyl)piperidin-4-yl)amino)-2-(3-((5-fluoro- |
| | 2-methoxy-4-(methylsulfonyl)phenyl)amino)prop-1-yn-1-yl)benzo[b]thiophene 1-oxide; |
| 1364 | 3-(cyclopropylmethyl)-7-(((3R,4R)-3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1-oxide; |
| 1365 | 3-(cyclopropylmethyl)-7-(((3S,4S)-3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1-oxide; |
| 1366 | 3-(cyclopropylmethyl)-7-((4-(dimethylamino)cyclohexyl)amino)-2-(3-((5-fluoro-2-methoxy-4- |
| | (methylsulfonyl)phenyl)amino)prop-1-yn-1-yl)benzo[b]thiophene 1-oxide; |
| 1367 | 3-(cyclopropylmethyl)-7-((2-(dimethylamino)ethyl)amino)-2-(3-((5-fluoro-2-methoxy-4- |
| | (methylsulfonyl)phenyl)amino)prop-1-yn-1-yl)benzo[b]thiophene 1-oxide; |
| 1368 | 3-(cyclopropylmethyl)-7-(((3S,4R)-3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1-oxide; |
| 1369 | 3-(cyclopropylmethyl)-2-(3-((5-fluoro-2-methoxy-4-(methylsulfonyl)phenyl)amino)prop-1-yn-1-yl)-7-((1- |
| | methylpiperidin-4-yl)amino)benzo[b]thiophene 1-oxide; |
| 1370 | 3-(cyclopropylmethyl)-2-(3-((5-fluoro-2-methoxy-4-(methylsulfonyl)phenyl)amino)prop-1-yn-1-yl)-7- |
| | ((tetrahydro-2H-pyran-4-yl)amino)benzo[b]thiophene 1-oxide; |
| 1371 | 7-((3,3-difluoro-1-methylpiperidin-4-yl)amino)-3-(2,2-difluoroethyl)-2-(3-((5-fluoro-2-methoxy-4- |
| | (methylsulfonyl)phenyl)amino)prop-1-yn-1-yl)benzo[b]thiophene 1-oxide; |
| 1372 | 4-((3-(2,2-difluoroethyl)-2-(3-((5-fluoro-2-methoxy-4-(methylsulfonyl)phenyl)amino)prop-1-yn-1-yl)-1- |
| | oxidobenzo[b]thiophen-7-yl)amino)tetrahydro-2H-thiopyran 1,1-dioxide; |
| 1373 | 3-(2,2-difluoroethyl)-7-(((3R,4S)-3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1-oxide; |
| 1374 | 3-(2,2-difluoroethyl)-7-((3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1-oxide; |
| 1375 | 1-(4-((3-(2,2-difluoroethyl)-2-(3-((5-fluoro-2-methoxy-4-(methylsulfonyl)phenyl)amino)prop-1-yn-1-yl)-1- |
| | oxidobenzo[b]thiophen-7-yl)amino)piperidin-1-yl)-2-(dimethylamino)ethan-1-one; |
| 1376 | 3-(2,2-difluoroethyl)-7-((3-fluoro-1-(2-hydroxy-3-methoxypropyl)piperidin-4-yl)amino)-2-(3-((5-fluoro-2- |
| | methoxy-4-(methylsulfonyl)phenyl)amino)prop-1-yn-1-yl)benzo[b]thiophene 1-oxide; |
| 1377 | 3-(2,2-difluoroethyl)-7-(((3R,4R)-3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1-oxide; |
| 1378 | 3-(2,2-difluoroethyl)-7-(((3S,4S)-3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1-oxide; |
| 1379 | 3-(2,2-difluoroethyl)-7-((4-(dimethylamino)cyclohexyl)amino)-2-(3-((5-fluoro-2-methoxy-4- |
| | (methylsulfonyl)phenyl)amino)prop-1-yn-1-yl)benzo[b]thiophene 1-oxide; |
| 1380 | 3-(2,2-difluoroethyl)-7-((2-(dimethylamino)ethyl)amino)-2-(3-((5-fluoro-2-methoxy-4- |
| | (methylsulfonyl)phenyl)amino)prop-1-yn-1-yl)benzo[b]thiophene 1-oxide; |
| 1381 | 3-(2,2-difluoroethyl)-7-(((3S,4R)-3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1-oxide; |
| 1382 | 3-(2,2-difluoroethyl)-2-(3-((5-fluoro-2-methoxy-4-(methylsulfonyl)phenyl)amino)prop-1-yn-1-yl)-7-((1- |
| | methylpiperidin-4-yl)amino)benzo[b]thiophene 1-oxide; |
| 1383 | 3-(2,2-difluoroethyl)-2-(3-((5-fluoro-2-methoxy-4-(methylsulfonyl)phenyl)amino)prop-1-yn-1-yl)-7-((1- |
| | ((tetrahydro-2H-pyran-4-yl)amino)benzo[b]thiophene 1-oxide; |
| 1384 | 7-((3,3-difluoro-1-methylpiperidin-4-yl)amino)-2-(3-((5-fluoro-2-methoxy-4- |
| | (methylsulfonyl)phenyl)amino)prop-1-yn-1-yl)-3-(1H-pyrrol-1-yl)benzo[b]thiophene 1-oxide; |
| 1385 | 4-((2-(3-((5-fluoro-2-methoxy-4-(methylsulfonyl)phenyl)amino)prop-1-yn-1-yl)-1-oxido-3-(1H-pyrrol-1- |
| | yl)benzo[b]thiophen-7-yl)amino)tetrahydro-2H-thiopyran 1,1-dioxide; |
| 1386 | 7-(((3R,4S)-3-fluoro-1-methylpiperidin-4-yl)amino)-2-(3-((5-fluoro-2-methoxy-4- |
| | (methylsulfonyl)phenyl)amino)prop-1-yn-1-yl)-3-(1H-pyrrol-1-yl)benzo[b]thiophene 1-oxide; |
| 1387 | 7-((3-fluoro-1-methylpiperidin-4-yl)amino)-2-(3-((5-fluoro-2-methoxy-4- |
| | (methylsulfonyl)phenyl)amino)prop-1-yn-1-yl)-3-(1H-pyrrol-1-yl)benzo[b]thiophene 1-oxide; |
| 1388 | 2-(dimethylamino)-1-(4-((2-(3-((5-fluoro-2-methoxy-4-(methylsulfonyl)phenyl)amino)prop-1-yn-1-yl)-1- |
| | oxido-3-(1H-pyrrol-1-yl)benzo[b]thiophen-7-yl)amino)piperidin-1-yl)ethan-1-one; |
| 1389 | 7-((3-fluoro-1-(2-hydroxy-3-methoxypropyl)piperidin-4-yl)amino)-2-(3-((5-fluoro-2-methoxy-4- |
| | (methylsulfonyl)phenyl)amino)prop-1-yn-1-yl)-3-(1H-pyrrol-1-yl)benzo[b]thiophene 1-oxide; |
| 1390 | 7-(((3R,4R)-3-fluoro-1-methylpiperidin-4-yl)amino)-2-(3-((5-fluoro-2-methoxy-4- |
| | (methylsulfonyl)phenyl)amino)prop-1-yn-1-yl)-3-(1H-pyrrol-1-yl)benzo[b]thiophene 1-oxide; |
| 1391 | 7-(((3S,4S)-3-fluoro-1-methylpiperidin-4-yl)amino)-2-(3-((5-fluoro-2-methoxy-4- |
| | (methylsulfonyl)phenyl)amino)prop-1-yn-1-yl)-3-(1H-pyrrol-1-yl)benzo[b]thiophene 1-oxide; |
| 1392 | 7-((4-(dimethylamino)cyclohexyl)amino)-2-(3-((5-fluoro-2-methoxy-4- |
| | (methylsulfonyl)phenyl)amino)prop-1-yn-1-yl)-3-(1H-pyrrol-1-yl)benzo[b]thiophene 1-oxide; |
| 1393 | 7-((2-(dimethylamino)ethyl)amino)-2-(3-((5-fluoro-2-methoxy-4-(methylsulfonyl)phenyl)amino)prop-1-yn- |
| | 1-yl)-3-(1H-pyrrol-1-yl)benzo[b]thiophene 1-oxide; |
| 1394 | 7-(((3S,4R)-3-fluoro-1-methylpiperidin-4-yl)amino)-2-(3-((5-fluoro-2-methoxy-4- |
| | (methylsulfonyl)phenyl)amino)prop-1-yn-1-yl)-3-(1H-pyrrol-1-yl)benzo[b]thiophene 1-oxide; |
| 1395 | 2-(3-((5-fluoro-2-methoxy-4-(methylsulfonyl)phenyl)amino)prop-1-yn-1-yl)-7-((1-methylpiperidin-4- |
| | yl)amino)-3-(1H-pyrrol-1-yl)benzo[b]thiophene 1-oxide; |
| 1396 | 2-(3-((5-fluoro-2-methoxy-4-(methylsulfonyl)phenyl)amino)prop-1-yn-1-yl)-3-(1H-pyrrol-1-yl)-7- |
| | ((tetrahydro-2H-pyran-4-yl)amino)benzo[b]thiophene 1-oxide; |
| 1397 | 7-((3,3-difluoro-1-methylpiperidin-4-yl)amino)-3-ethyl-2-(3-((5-fluoro-2-methoxy-4- |
| | (methylsulfonyl)phenyl)amino)prop-1-yn-1-yl)benzo[b]thiophene 1-oxide; |
| 1398 | 4-((3-ethyl-2-(3-((5-fluoro-2-methoxy-4-(methylsulfonyl)phenyl)amino)prop-1-yn-1-yl)-1- |
| | oxidobenzo[b]thiophen-7-yl)amino)tetrahydro-2H-thiopyran 1,1-dioxide; |
| 1399 | 3-ethyl-7-(((3R,4S)-3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1-oxide; |
| 1400 | 3-ethyl-7-((3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1-oxide; |
| 1401 | 2-(dimethylamino)-1-(4-((3-ethyl-2-(3-((5-fluoro-2-methoxy-4-(methylsulfonyl)phenyl)amino)prop-1-yn-1- |
| | yl)-1-oxidobenzo[b]thiophen-7-yl)amino)piperidin-1-yl)ethan-1-one; |
| 1402 | 3-ethyl-7-((3-fluoro-1-(2-hydroxy-3-methoxypropyl)piperidin-4-yl)amino)-2-(3-((5-fluoro-2-methoxy-4- |
| | (methylsulfonyl)phenyl)amino)prop-1-yn-1-yl)benzo[b]thiophene 1-oxide; |
| 1403 | 3-ethyl-7-(((3R,4R)-3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1-oxide; |
| 1404 | 3-ethyl-7-(((3S,4S)-3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1-oxide; |
| 1405 | 7-((4-(dimethylamino)cyclohexyl)amino)-3-ethyl-2-(3-((5-fluoro-2-methoxy-4- |
| | (methylsulfonyl)phenyl)amino)prop-1-yn-1-yl)benzo[b]thiophene 1-oxide; |
| 1406 | 7-((2-(dimethylamino)ethyl)amino)-3-ethyl-2-(3-((5-fluoro-2-methoxy-4- |
| | (methylsulfonyl)phenyl)amino)prop-1-yn-1-yl)benzo[b]thiophene 1-oxide; |
| 1407 | 3-ethyl-7-(((3S,4R)-3-fluoro-1-methylpiperidin-4-yl)amino)-2-(3-((5-fluoro-2-methoxy-4- |
| | (methylsulfonyl)phenyl)amino)prop-1-yn-1-yl)benzo[b]thiophene 1-oxide; |
| 1408 | 3-ethyl-2-(3-((5-fluoro-2-methoxy-4-(methylsulfonyl)phenyl)amino)prop-1-yn-1-yl)-7-((1-methylpiperidin- |
| | 4-yl)amino)benzo[b]thiophene 1-oxide; |
| 1409 | 3-ethyl-2-(3-((5-fluoro-2-methoxy-4-(methylsulfonyl)phenyl)amino)prop-1-yn-1-yl)-7-((tetrahydro-2H- |
| | pyran-4-yl)amino)benzo[b]thiophene 1-oxide; |
| 1410 | 4-((3-(3-(cyanomethyl)-7-((3,3-difluoro-1-methylpiperidin-4-yl)amino)-1-oxidobenzo[b]thiophen-2- |
| | yl)prop-2-yn-1-yl)amino)-3-methoxy-N-methylbenzamide; |
| 1411 | 4-((3-(3-(cyanomethyl)-7-((1,1-dioxidotetrahydro-2H-thiopyran-4-yl)amino)-1-oxidobenzo[b]thiophen-2- |
| | yl)prop-2-yn-1-yl)amino)-3-methoxy-N-methylbenzamide; |
| 1412 | 4-((3-(3-(cyanomethyl)-7-(((3R,4S)-3-fluoro-1-methylpiperidin-4-yl)amino)-1-oxidobenzo[b]thiophen-2- |
| | yl)prop-2-yn-1-yl)amino)-3-methoxy-N-methylbenzamide; |
| 1413 | 4-((3-(3-(cyanomethyl)-7-((3-fluoro-1-methylpiperidin-4-yl)amino)-1-oxidobenzo[b]thiophen-2-yl)prop-2- |
| | yn-1-yl)amino)-3-methoxy-N-methylbenzamide; |
| 1414 | 4-((3-(3-(cyanomethyl)-7-((1-(dimethylglycyl)piperidin-4-yl)amino)-1-oxidobenzo[b]thiophen-2-yl)prop-2- |
| | yn-1-yl)amino)-3-methoxy-N-methylbenzamide; |
| 1415 | 4-((3-(3-(cyanomethyl)-7-((3-fluoro-1-(2-hydroxy-3-methoxypropyl)piperidin-4-yl)amino)-1- |
| | oxidobenzo[b]thiophen-2-yl)prop-2-yn-1-yl)amino)-3-methoxy-N-methylbenzamide; |
| 1416 | 4-((3-(3-(cyanomethyl)-7-(((3R,4R)-3-fluoro-1-methylpiperidin-4-yl)amino)-1-oxidobenzo[b]thiophen-2- |
| | yl)prop-2-yn-1-yl)amino)-3-methoxy-N-methylbenzamide; |
| 1417 | 4-((3-(3-(cyanomethyl)-7-(((3S,4S)-3-fluoro-1-methylpiperidin-4-yl)amino)-1-oxidobenzo[b]thiophen-2- |
| | yl)prop-2-yn-1-yl)amino)-3-methoxy-N-methylbenzamide; |
| 1418 | 4-((3-(3-(cyanomethyl)-7-((4-(dimethylamino)cyclohexyl)amino)-1-oxidobenzo[b]thiophen-2-yl)prop-2-yn- |
| | 1-yl)amino)-3-methoxy-N-methylbenzamide; |
| 1419 | 4-((3-(3-(cyanomethyl)-7-((2-(dimethylamino)ethyl)amino)-1-oxidobenzo[b]thiophen-2-yl)prop-2-yn-1- |
| | yl)amino)-3-methoxy-N-methylbenzamide; |
| 1420 | 4-((3-(3-(cyanomethyl)-7-(((3S,4R)-3-fluoro-1-methylpiperidin-4-yl)amino)-1-oxidobenzo[b]thiophen-2- |
| | yl)prop-2-yn-1-yl)amino)-3-methoxy-N-methylbenzamide; |
| 1421 | 4-((3-(3-(cyanomethyl)-7-((1-methylpiperidin-4-yl)amino)-1-oxidobenzo[b]thiophen-2-yl)prop-2-yn-1- |
| | yl)amino)-3-methoxy-N-methylbenzamide; |
| 1422 | 4-((3-(3-(cyanomethyl)-1-oxido-7-((tetrahydro-2H-pyran-4-yl)amino)benzo[b]thiophen-2-yl)prop-2-yn-1- |
| | yl)amino)-3-methoxy-N-methylbenzamide; |
| 1423 | 4-((3-(7-((3,3-difluoro-1-methylpiperidin-4-yl)amino)-1-oxido-3-(thiazol-4-yl)benzo[b]thiophen-2-yl)prop- |
| | 2-yn-1-yl)amino)-3-methoxy-N-methylbenzamide; |
| 1424 | 4-((3-(7-((1,1-dioxidotetrahydro-2H-thiopyran-4-yl)amino)-1-oxido-3-(thiazol-4-yl)benzo[b]thiophen-2- |
| | yl)prop-2-yn-1-yl)amino)-3-methoxy-N-methylbenzamide; |
| 1425 | 4-((3-(7-(((3R,4S)-3-fluoro-1-methylpiperidin-4-yl)amino)-1-oxido-3-(thiazol-4-yl)benzo[b]thiophen-2- |
| | yl)prop-2-yn-1-yl)amino)-3-methoxy-N-methylbenzamide; |
| 1426 | 4-((3-(7-((3-fluoro-1-methylpiperidin-4-yl)amino)-1-oxido-3-(thiazol-4-yl)benzo[b]thiophen-2-yl)prop-2- |
| | yn-1-yl)amino)-3-methoxy-N-methylbenzamide; |
| 1427 | 4-((3-(7-((1-(dimethylglycyl)piperidin-4-yl)amino)-1-oxido-3-(thiazol-4-yl)benzo[b]thiophen-2-yl)prop-2- |
| | yn-1-yl)amino)-3-methoxy-N-methylbenzamide; |
| 1428 | 4-((3-(7-((3-fluoro-1-(2-hydroxy-3-methoxypropyl)piperidin-4-yl)amino)-1-oxido-3-(thiazol-4- |
| | yl)benzo[b]thiophen-2-yl)prop-2-yn-1-yl)amino)-3-methoxy-N-methylbenzamide; |
| 1429 | 4-((3-(7-(((3R,4R)-3-fluoro-1-methylpiperidin-4-yl)amino)-1-oxido-3-(thiazol-4-yl)benzo[b]thiophen-2- |
| | yl)prop-2-yn-1-yl)amino)-3-methoxy-N-methylbenzamide; |
| 1430 | 4-((3-(7-(((3S,4S)-3-fluoro-1-methylpiperidin-4-yl)amino)-1-oxido-3-(thiazol-4-yl)benzo[b]thiophen-2- |
| | yl)prop-2-yn-1-yl)amino)-3-methoxy-N-methylbenzamide; |
| 1431 | 4-((3-(7-((4-(dimethylamino)cyclohexyl)amino)-1-oxido-3-(thiazol-4-yl)benzo[b]thiophen-2-yl)prop-2-yn- |
| | 1-yl)amino)-3-methoxy-N-methylbenzamide; |
| 1432 | 4-((3-(7-((2-(dimethylamino)ethyl)amino)-1-oxido-3-(thiazol-4-yl)benzo[b]thiophen-2-yl)prop-2-yn-1- |
| | yl)amino)-3-methoxy-N-methylbenzamide; |
| 1433 | 4-((3-(7-(((3S,4R)-3-fluoro-1-methylpiperidin-4-yl)amino)-1-oxido-3-(thiazol-4-yl)benzo[b]thiophen-2- |
| | yl)prop-2-yn-1-yl)amino)-3-methoxy-N-methylbenzamide; |
| 1434 | 3-methoxy-N-methyl-4-((3-(7-((1-methylpiperidin-4-yl)amino)-1-oxido-3-(thiazol-4-yl)benzo[b]thiophen- |
| | 2-yl)prop-2-yn-1-yl)amino)benzamide; |
| 1435 | 3-methoxy-N-methyl-4-((3-(1-oxido-7-((tetrahydro-2H-pyran-4-yl)amino)-3-(thiazol-4- |
| | yl)benzo[b]thiophen-2-yl)prop-2-yn-1-yl)amino)benzamide; |
| 1436 | 4-((3-(7-((3,3-difluoro-1-methylpiperidin-4-yl)amino)-1-oxido-3-(2,2,2-trifluoroethyl)benzo[b]thiophen-2- |
| | yl)prop-2-yn-1-yl)amino)-3-methoxy-N-methylbenzamide; |
| 1437 | 4-((3-(7-((1,1-dioxidotetrahydro-2H-thiopyran-4-yl)amino)-1-oxido-3-(2,2,2- |
| | trifluoroethyl)benzo[b]thiophen-2-yl)prop-2-yn-1-yl)amino)-3-methoxy-N-methylbenzamide; |
| 1438 | 4-((3-(7-(((3R,4S)-3-fluoro-1-methylpiperidin-4-yl)amino)-1-oxido-3-(2,2,2- |
| | trifluoroethyl)benzo[b]thiophen-2-yl)prop-2-yn-1-yl)amino)-3-methoxy-N-methylbenzamide; |
| 1439 | 4-((3-(7-((3-fluoro-1-methylpiperidin-4-yl)amino)-1-oxido-3-(2,2,2-trifluoroethyl)benzo[b]thiophen-2- |
| | yl)prop-2-yn-1-yl)amino)-3-methoxy-N-methylbenzamide; |
| 1440 | 4-((3-(7-((1-(dimethylglycyl)piperidin-4-yl)amino)-1-oxido-3-(2,2,2-trifluoroethyl)benzo[b]thiophen-2- |
| | yl)prop-2-yn-1-yl)amino)-3-methoxy-N-methylbenzamide; |
| 1441 | 4-((3-(7-((3-fluoro-1-(2-hydroxy-3-methoxypropyl)piperidin-4-yl)amino)-1-oxido-3-(2,2,2- |
| | trifluoroethyl)benzo[b]thiophen-2-yl)prop-2-yn-1-yl)amino)-3-methoxy-N-methylbenzamide; |
| 1442 | 4-((3-(7-(((3R,4R)-3-fluoro-1-methylpiperidin-4-yl)amino)-1-oxido-3-(2,2,2- |
| | trifluoroethyl)benzo[b]thiophen-2-yl)prop-2-yn-1-yl)amino)-3-methoxy-N-methylbenzamide; |
| 1443 | 4-((3-(7-(((3S,4S)-3-fluoro-1-methylpiperidin-4-yl)amino)-1-oxido-3-(2,2,2- |
| | trifluoroethyl)benzo[b]thiophen-2-yl)prop-2-yn-1-yl)amino)-3-methoxy-N-methylbenzamide; |
| 1444 | 4-((3-(7-((4-(dimethylamino)cyclohexyl)amino)-1-oxido-3-(2,2,2-trifluoroethyl)benzo[b]thiophen-2- |
| | yl)prop-2-yn-1-yl)amino)-3-methoxy-N-methylbenzamide; |
| 1445 | 4-((3-(7-((2-(dimethylamino)ethyl)amino)-1-oxido-3-(2,2,2-trifluoroethyl)benzo[b]thiophen-2-yl)prop-2- |
| | yn-1-yl)amino)-3-methoxy-N-methylbenzamide; |
| 1446 | 4-((3-(7-(((3S,4R)-3-fluoro-1-methylpiperidin-4-yl)amino)-1-oxido-3-(2,2,2- |
| | trifluoroethyl)benzo[b]thiophen-2-yl)prop-2-yn-1-yl)amino)-3-methoxy-N-methylbenzamide; |
| 1447 | 3-methoxy-N-methyl-4-((3-(7-((1-methylpiperidin-4-yl)amino)-1-oxido-3-(2,2,2- |
| | trifluoroethyl)benzo[b]thiophen-2-yl)prop-2-yn-1-yl)amino)benzamide; |
| 1448 | 3-methoxy-N-methyl-4-((3-(1-oxido-7-((tetrahydro-2H-pyran-4-yl)amino)-3-(2,2,2- |
| | trifluoroethyl)benzo[b]thiophen-2-yl)prop-2-yn-1-yl)amino)benzamide; |
| 1449 | 4-((3-(3-(cyclopropylmethyl)-7-((3,3-difluoro-1-methylpiperidin-4-yl)amino)-1-oxidobenzo[b]thiophen-2- |
| | yl)prop-2-yn-1-yl)amino)-3-methoxy-N-methylbenzamide; |
| 1450 | 4-((3-(3-(cyclopropylmethyl)-7-((1,1-dioxidotetrahydro-2H-thiopyran-4-yl)amino)-1- |
| | oxidobenzo[b]thiophen-2-yl)prop-2-yn-1-yl)amino)-3-methoxy-N-methylbenzamide; |
| 1451 | 4-((3-(3-(cyclopropylmethyl)-7-(((3R,4S)-3-fluoro-1-methylpiperidin-4-yl)amino)-1- |
| | oxidobenzo[b]thiophen-2-yl)prop-2-yn-1-yl)amino)-3-methoxy-N-methylbenzamide; |
| 1452 | 4-((3-(3-(cyclopropylmethyl)-7-((3-fluoro-1-methylpiperidin-4-yl)amino)-1-oxidobenzo[b]thiophen-2- |
| | yl)prop-2-yn-1-yl)amino)-3-methoxy-N-methylbenzamide; |
| 1453 | 4-((3-(3-(cyclopropylmethyl)-7-((1-(dimethylglycyl)piperidin-4-yl)amino)-1-oxidobenzo[b]thiophen-2- |
| | yl)prop-2-yn-1-yl)amino)-3-methoxy-N-methylbenzamide; |
| 1454 | 4-((3-(3-(cyclopropylmethyl)-7-((3-fluoro-1-(2-hydroxy-3-methoxypropyl)piperidin-4-yl)amino)-1- |
| | oxidobenzo[b]thiophen-2-yl)prop-2-yn-1-yl)amino)-3-methoxy-N-methylbenzamide; |
| 1455 | 4-((3-(3-(cyclopropylmethyl)-7-(((3R,4R)-3-fluoro-1-methylpiperidin-4-yl)amino)-1- |
| | oxidobenzo[b]thiophen-2-yl)prop-2-yn-1-yl)amino)-3-methoxy-N-methylbenzamide; |
| 1456 | 4-((3-(3-(cyclopropylmethyl)-7-(((3S,4S)-3-fluoro-1-methylpiperidin-4-yl)amino)-1- |
| | oxidobenzo[b]thiophen-2-yl)prop-2-yn-1-yl)amino)-3-methoxy-N-methylbenzamide; |
| 1457 | 4-((3-(3-(cyclopropylmethyl)-7-((4-(dimethylamino)cyclohexyl)amino)-1-oxidobenzo[b]thiophen-2- |
| | yl)prop-2-yn-1-yl)amino)-3-methoxy-N-methylbenzamide; |
| 1458 | 4-((3-(3-(cyclopropylmethyl)-7-((2-(dimethylamino)ethyl)amino)-1-oxidobenzo[b]thiophen-2-yl)prop-2-yn- |
| | 1-yl)amino)-3-methoxy-N-methylbenzamide; |
| 1459 | 4-((3-(3-(cyclopropylmethyl)-7-(((3S,4R)-3-fluoro-1-methylpiperidin-4-yl)amino)-1- |
| | oxidobenzo[b]thiophen-2-yl)prop-2-yn-1-yl)amino)-3-methoxy-N-methylbenzamide; |
| 1460 | 4-((3-(3-(cyclopropylmethyl)-7-((1-methylpiperidin-4-yl)amino)-1-oxidobenzo[b]thiophen-2-yl)prop-2-yn- |
| | 1-yl)amino)-3-methoxy-N-methylbenzamide; |
| 1461 | 4-((3-(3-(cyclopropylmethyl)-1-oxido-7-((tetrahydro-2H-pyran-4-yl)amino)benzo[b]thiophen-2-yl)prop-2- |
| | yn-1-yl)amino)-3-methoxy-N-methylbenzamide; |
| 1462 | 4-((3-(7-((3,3-difluoro-1-methylpiperidin-4-yl)amino)-3-(2,2-difluoroethyl)-1-oxidobenzo[b]thiophen-2- |
| | yl)prop-2-yn-1-yl)amino)-3-methoxy-N-methylbenzamide; |
| 1463 | 4-((3-(3-(2,2-difluoroethyl)-7-((1,1-dioxidotetrahydro-2H-thiopyran-4-yl)amino)-1-oxidobenzo[b]thiophen- |
| | 2-yl)prop-2-yn-1-yl)amino)-3-methoxy-N-methylbenzamide; |
| 1464 | 4-((3-(3-(2,2-difluoroethyl)-7-(((3R,4S)-3-fluoro-1-methylpiperidin-4-yl)amino)-1-oxidobenzo[b]thiophen- |
| | 2-yl)prop-2-yn-1-yl)amino)-3-methoxy-N-methylbenzamide; |
| 1465 | 4-((3-(3-(2,2-difluoroethyl)-7-((3-fluoro-1-methylpiperidin-4-yl)amino)-1-oxidobenzo[b]thiophen-2- |
| | yl)prop-2-yn-1-yl)amino)-3-methoxy-N-methylbenzamide; |
| 1466 | 4-((3-(3-(2,2-difluoroethyl)-7-((1-(dimethylglycyl)piperidin-4-yl)amino)-1-oxidobenzo[b]thiophen-2- |
| | yl)prop-2-yn-1-yl)amino)-3-methoxy-N-methylbenzamide; |
| 1467 | 4-((3-(3-(2,2-difluoroethyl)-7-((3-fluoro-1-(2-hydroxy-3-methoxypropyl)piperidin-4-yl)amino)-1- |
| | oxidobenzo[b]thiophen-2-yl)prop-2-yn-1-yl)amino)-3-methoxy-N-methylbenzamide; |
| 1468 | 4-((3-(3-(2,2-difluoroethyl)-7-(((3R,4R)-3-fluoro-1-methylpiperidin-4-yl)amino)-1-oxidobenzo[b]thiophen- |
| | 2-yl)prop-2-yn-1-yl)amino)-3-methoxy-N-methylbenzamide; |
| 1469 | 4-((3-(3-(2,2-difluoroethyl)-7-(((3S,4S)-3-fluoro-1-methylpiperidin-4-yl)amino)-1-oxidobenzo[b]thiophen- |
| | 2-yl)prop-2-yn-1-yl)amino)-3-methoxy-N-methylbenzamide; |
| 1470 | 4-((3-(3-(2,2-difluoroethyl)-7-((4-(dimethylamino)cyclohexyl)amino)-1-oxidobenzo[b]thiophen-2-yl)prop- |
| | 2-yn-1-yl)amino)-3-methoxy-N-methylbenzamide; |
| 1471 | 4-((3-(3-(2,2-difluoroethyl)-7-((2-(dimethylamino)ethyl)amino)-1-oxidobenzo[b]thiophen-2-yl)prop-2-yn- |
| | 1-yl)amino)-3-methoxy-N-methylbenzamide; |
| 1472 | 4-((3-(3-(2,2-difluoroethyl)-7-(((3S,4R)-3-fluoro-1-methylpiperidin-4-yl)amino)-1-oxidobenzo[b]thiophen- |
| | 2-yl)prop-2-yn-1-yl)amino)-3-methoxy-N-methylbenzamide; |
| 1473 | 4-((3-(3-(2,2-difluoroethyl)-7-((1-methylpiperidin-4-yl)amino)-1-oxidobenzo[b]thiophen-2-yl)prop-2-yn-1- |
| | yl)amino)-3-methoxy-N-methylbenzamide; |
| 1474 | 4-((3-(3-(2,2-difluoroethyl)-1-oxido-7-((tetrahydro-2H-pyran-4-yl)amino)benzo[b]thiophen-2-yl)prop-2-yn- |
| | 1-yl)amino)-3-methoxy-N-methylbenzamide; |
| 1475 | 4-((3-(7-((3,3-difluoro-1-methylpiperidin-4-yl)amino)-1-oxido-3-(1H-pyrrol-1-yl)benzo[b]thiophen-2- |
| | yl)prop-2-yn-1-yl)amino)-3-methoxy-N-methylbenzamide; |
| 1476 | 4-((3-(7-((1,1-dioxidotetrahydro-2H-thiopyran-4-yl)amino)-1-oxido-3-(1H-pyrrol-1-yl)benzo[b]thiophen-2- |
| | yl)prop-2-yn-1-yl)amino)-3-methoxy-N-methylbenzamide; |
| 1477 | 4-((3-(7-(((3R,4S)-3-fluoro-1-methylpiperidin-4-yl)amino)-1-oxido-3-(1H-pyrrol-1-yl)benzo[b]thiophen-2- |
| | yl)prop-2-yn-1-yl)amino)-3-methoxy-N-methylbenzamide; |
| 1478 | 4-((3-(7-((3-fluoro-1-methylpiperidin-4-yl)amino)-1-oxido-3-(1H-pyrrol-1-yl)benzo[b]thiophen-2-yl)prop- |
| | 2-yn-1-yl)amino)-3-methoxy-N-methylbenzamide; |
| 1479 | 4-((3-(7-((1-(dimethylglycyl)piperidin-4-yl)amino)-1-oxido-3-(1H-pyrrol-1-yl)benzo[b]thiophen-2-yl)prop- |
| | 2-yn-1-yl)amino)-3-methoxy-N-methylbenzamide; |
| 1480 | 4-((3-(7-((3-fluoro-1-(2-hydroxy-3-methoxypropyl)piperidin-4-yl)amino)-1-oxido-3-(1H-pyrrol-1- |
| | yl)benzo[b]thiophen-2-yl)prop-2-yn-1-yl)amino)-3-methoxy-N-methylbenzamide; |
| 1481 | 4-((3-(7-(((3R,4R)-3-fluoro-1-methylpiperidin-4-yl)amino)-1-oxido-3-(1H-pyrrol-1-yl)benzo[b]thiophen-2- |
| | yl)prop-2-yn-1-yl)amino)-3-methoxy-N-methylbenzamide; |
| 1482 | 4-((3-(7-(((3S,4S)-3-fluoro-1-methylpiperidin-4-yl)amino)-1-oxido-3-(1H-pyrrol-1-yl)benzo[b]thiophen-2- |
| | yl)prop-2-yn-1-yl)amino)-3-methoxy-N-methylbenzamide; |
| 1483 | 4-((3-(7-((4-(dimethylamino)cyclohexyl)amino)-1-oxido-3-(1H-pyrrol-1-yl)benzo[b]thiophen-2-yl)prop-2- |
| | yn-1-yl)amino)-3-methoxy-N-methylbenzamide; |
| 1484 | 4-((3-(7-((2-(dimethylamino)ethyl)amino)-1-oxido-3-(1H-pyrrol-1-yl)benzo[b]thiophen-2-yl)prop-2-yn-1- |
| | yl)amino)-3-methoxy-N-methylbenzamide; |
| 1485 | 4-((3-(7-(((3S,4R)-3-fluoro-1-methylpiperidin-4-yl)amino)-1-oxido-3-(1H-pyrrol-1-yl)benzo[b]thiophen-2- |
| | yl)prop-2-yn-1-yl)amino)-3-methoxy-N-methylbenzamide; |
| 1486 | 3-methoxy-N-methyl-4-((3-(7-((1-methylpiperidin-4-yl)amino)-1-oxido-3-(1H-pyrrol-1- |
| | yl)benzo[b]thiophen-2-yl)prop-2-yn-1-yl)amino)benzamide; |
| 1487 | 3-methoxy-N-methyl-4-((3-(1-oxido-3-(1H-pyrrol-1-yl)-7-((tetrahydro-2H-pyran-4- |
| | yl)amino)benzo[b]thiophen-2-yl)prop-2-yn-1-yl)amino)benzamide; |
| 1488 | 4-((3-(7-((3,3-difluoro-1-methylpiperidin-4-yl)amino)-3-ethyl-1-oxidobenzo[b]thiophen-2-yl)prop-2-yn-1- |
| | yl)amino)-3-methoxy-N-methylbenzamide; |
| 1489 | 4-((3-(7-((1,1-dioxidotetrahydro-2H-thiopyran-4-yl)amino)-3-ethyl-1-oxidobenzo[b]thiophen-2-yl)prop-2- |
| | yn-1-yl)amino)-3-methoxy-N-methylbenzamide; |
| 1490 | 4-((3-(3-ethyl-7-(((3R,4S)-3-fluoro-1-methylpiperidin-4-yl)amino)-1-oxidobenzo[b]thiophen-2-yl)prop-2- |
| | yn-1-yl)amino)-3-methoxy-N-methylbenzamide; |
| 1491 | 4-((3-(3-ethyl-7-((3-fluoro-1-methylpiperidin-4-yl)amino)-1-oxidobenzo[b]thiophen-2-yl)prop-2-yn-1- |
| | yl)amino)-3-methoxy-N-methylbenzamide; |
| 1492 | 4-((3-(7-((1-(dimethylglycyl)piperidin-4-yl)amino)-3-ethyl-1-oxidobenzo[b]thiophen-2-yl)prop-2-yn-1- |
| | yl)amino)-3-methoxy-N-methylbenzamide; |
| 1493 | 4-((3-(3-ethyl-7-((3-fluoro-1-(2-hydroxy-3-methoxypropyl)piperidin-4-yl)amino)-1- |
| | oxidobenzo[b]thiophen-2-yl)prop-2-yn-1-yl)amino)-3-methoxy-N-methylbenzamide; |
| 1494 | 4-((3-(3-ethyl-7-(((3R,4R)-3-fluoro-1-methylpiperidin-4-yl)amino)-1-oxidobenzo[b]thiophen-2-yl)prop-2- |
| | yn-1-yl)amino)-3-methoxy-N-methylbenzamide; |
| 1495 | 4-((3-(3-ethyl-7-(((3S,4S)-3-fluoro-1-methylpiperidin-4-yl)amino)-1-oxidobenzo[b]thiophen-2-yl)prop-2- |
| | yn-1-yl)amino)-3-methoxy-N-methylbenzamide; |
| 1496 | 4-((3-(7-((4-(dimethylamino)cyclohexyl)amino)-3-ethyl-1-oxidobenzo[b]thiophen-2-yl)prop-2-yn-1- |
| | yl)amino)-3-methoxy-N-methylbenzamide; |
| 1497 | 4-((3-(7-((2-(dimethylamino)ethyl)amino)-3-ethyl-1-oxidobenzo[b]thiophen-2-yl)prop-2-yn-1-yl)amino)-3- |
| | methoxy-N-methylbenzamide; |
| 1498 | 4-((3-(3-ethyl-7-(((3S,4R)-3-fluoro-1-methylpiperidin-4-yl)amino)-1-oxidobenzo[b]thiophen-2-yl)prop-2- |
| | yn-1-yl)amino)-3-methoxy-N-methylbenzamide; |
| 1499 | 4-((3-(3-ethyl-7-((1-methylpiperidin-4-yl)amino)-1-oxidobenzo[b]thiophen-2-yl)prop-2-yn-1-yl)amino)-3- |
| | methoxy-N-methylbenzamide; |
| 1500 | 4-((3-(3-ethyl-1-oxido-7-((tetrahydro-2H-pyran-4-yl)amino)benzo[b]thiophen-2-yl)prop-2-yn-1-yl)amino)- |
| | 3-methoxy-N-methylbenzamide; |
| 1501 | 2-(7-((3,3-difluoro-1-methylpiperidin-4-yl)amino)-2-(3-((2-methoxy-4- |
| | (methylsulfonyl)phenyl)amino)prop-1-yn-1-yl)-1-oxidobenzo[b]thiophen-3-yl)acetonitrile; |
| 1502 | 2-(7-((1,1-dioxidotetrahydro-2H-thiopyran-4-yl)amino)-2-(3-((2-methoxy-4- |
| | (methylsulfonyl)phenyl)amino)prop-1-yn-1-yl)-1-oxidobenzo[b]thiophen-3-yl)acetonitrile; |
| 1503 | 2-(7-(((3R,4S)-3-fluoro-1-methylpiperidin-4-yl)amino)-2-(3-((2-methoxy-4- |
| | (methylsulfonyl)phenyl)amino)prop-1-yn-1-yl)-1-oxidobenzo[b]thiophen-3-yl)acetonitrile; |
| 1504 | 2-(7-((3-fluoro-1-methylpiperidin-4-yl)amino)-2-(3-((2-methoxy-4-(methylsulfonyl)phenyl)amino)prop-1- |
| | yn-1-yl)-1-oxidobenzo[b]thiophen-3-yl)acetonitrile; |
| 1505 | 2-(7-((1-(dimethylglycyl)piperidin-4-yl)amino)-2-(3-((2-methoxy-4-(methylsulfonyl)phenyl)amino)prop-1- |
| | yn-1-yl)-1-oxidobenzo[b]thiophen-3-yl)acetonitrile; |
| 1506 | 2-(7-((3-fluoro-1-(2-hydroxy-3-methoxypropyl)piperidin-4-yl)amino)-2-(3-((2-methoxy-4- |
| | (methylsulfonyl)phenyl)amino)prop-1-yn-1-yl)-1-oxidobenzo[b]thiophen-3-yl)acetonitrile; |
| 1507 | 2-(7-(((3R,4R)-3-fluoro-1-methylpiperidin-4-yl)amino)-2-(3-((2-methoxy-4- |
| | (methylsulfonyl)phenyl)amino)prop-1-yn-1-yl)-1-oxidobenzo[b]thiophen-3-yl)acetonitrile; |
| 1508 | 2-(7-(((3S,4S)-3-fluoro-1-methylpiperidin-4-yl)amino)-2-(3-((2-methoxy-4- |
| | (methylsulfonyl)phenyl)amino)prop-1-yn-1-yl)-1-oxidobenzo[b]thiophen-3-yl)acetonitrile; |
| 1509 | 2-(7-((4-(dimethylamino)cyclohexyl)amino)-2-(3-((2-methoxy-4-(methylsulfonyl)phenyl)amino)prop-1-yn- |
| | 1-yl)-1-oxidobenzo[b]thiophen-3-yl)acetonitrile; |
| 1510 | 2-(7-((2-(dimethylamino)ethyl)amino)-2-(3-((2-methoxy-4-(methylsulfonyl)phenyl)amino)prop-1-yn-1-yl)- |
| | 1-oxidobenzo[b]thiophen-3-yl)acetonitrile; |
| 1511 | 2-(7-(((3S,4R)-3-fluoro-1-methylpiperidin-4-yl)amino)-2-(3-((2-methoxy-4- |
| | (methylsulfonyl)phenyl)amino)prop-1-yn-1-yl)-1-oxidobenzo[b]thiophen-3-yl)acetonitrile; |
| 1512 | 2-(2-(3-((2-methoxy-4-(methylsulfonyl)phenyl)amino)prop-1-yn-1-yl)-7-((1-methylpiperidin-4-yl)amino)- |
| | 1-oxidobenzo[b]thiophen-3-yl)acetonitrile; |
| 1513 | 2-(2-(3-((2-methoxy-4-(methylsulfonyl)phenyl)amino)prop-1-yn-1-yl)-1-oxido-7-((tetrahydro-2H-pyran-4- |
| | yl)amino)benzo[b]thiophen-3-yl)acetonitrile; |
| 1514 | 7-((3,3-difluoro-1-methylpiperidin-4-yl)amino)-2-(3-((2-methoxy-4-(methylsulfonyl)phenyl)amino)prop-1- |
| | yn-1-yl)-3-(thiazol-4-yl)benzo[b]thiophene 1-oxide; |
| 1515 | 4-((2-(3-((2-methoxy-4-(methylsulfonyl)phenyl)amino)prop-1-yn-1-yl)-1-oxido-3-(thiazol-4- |
| | yl)benzo[b]thiophen-7-yl)amino)tetrahydro-2H-thiopyran 1,1-dioxide; |
| 1516 | 7-(((3R,4S)-3-fluoro-1-methylpiperidin-4-yl)amino)-2-(3-((2-methoxy-4- |
| | (methylsulfonyl)phenyl)amino)prop-1-yn-1-yl)-3-(thiazol-4-yl)benzo[b]thiophene 1-oxide; |
| 1517 | 7-((3-fluoro-1-methylpiperidin-4-yl)amino)-2-(3-((2-methoxy-4-(methylsulfonyl)phenyl)amino)prop-1-yn- |
| | 1-yl)-3-(thiazol-4-yl)benzo[b]thiophene 1-oxide; |
| 1518 | 2-(dimethylamino)-1-(4-((2-(3-((2-methoxy-4-(methylsulfonyl)phenyl)amino)prop-1-yn-1-yl)-1-oxido-3- |
| | (thiazol-4-yl)benzo[b]thiophen-7-yl)amino)piperidin-1-yl)ethan-1-one; |
| 1519 | 7-((3-fluoro-1-(2-hydroxy-3-methoxypropyl)piperidin-4-yl)amino)-2-(3-((2-methoxy-4- |
| | (methylsulfonyl)phenyl)amino)prop-1-yn-1-yl)-3-(thiazol-4-yl)benzo[b]thiophene 1-oxide; |
| 1520 | 7-(((3R,4R)-3-fluoro-1-methylpiperidin-4-yl)amino)-2-(3-((2-methoxy-4- |
| | (methylsulfonyl)phenyl)amino)prop-1-yn-1-yl)-3-(thiazol-4-yl)benzo[b]thiophene 1-oxide; |
| 1521 | 7-(((3S,4S)-3-fluoro-1-methylpiperidin-4-yl)amino)-2-(3-((2-methoxy-4- |
| | (methylsulfonyl)phenyl)amino)prop-1-yn-1-yl)-3-(thiazol-4-yl)benzo[b]thiophene 1-oxide; |
| 1522 | 7-((4-(dimethylamino)cyclohexyl)amino)-2-(3-((2-methoxy-4-(methylsulfonyl)phenyl)amino)prop-1-yn-1- |
| | yl)-3-(thiazol-4-yl)benzo[b]thiophene 1-oxide; |
| 1523 | 7-((2-(dimethylamino)ethyl)amino)-2-(3-((2-methoxy-4-(methylsulfonyl)phenyl)amino)prop-1-yn-1-yl)-3- |
| | (thiazol-4-yl)benzo[b]thiophene 1-oxide; |
| 1524 | 7-(((3S,4R)-3-fluoro-1-methylpiperidin-4-yl)amino)-2-(3-((2-methoxy-4- |
| | (methylsulfonyl)phenyl)amino)prop-1-yn-1-yl)-3-(thiazol-4-yl)benzo[b]thiophene 1-oxide; |
| 1525 | 2-(3-((2-methoxy-4-(methylsulfonyl)phenyl)amino)prop-1-yn-1-yl)-7-((1-methylpiperidin-4-yl)amino)-3- |
| | (thiazol-4-yl)benzo[b]thiophene 1-oxide; |
| 1526 | 2-(3-((2-methoxy-4-(methylsulfonyl)phenyl)amino)prop-1-yn-1-yl)-7-((tetrahydro-2H-pyran-4-yl)amino)- |
| | 3-(thiazol-4-yl)benzo[b]thiophene 1-oxide; |
| 1527 | 7-((3,3-difluoro-1-methylpiperidin-4-yl)amino)-2-(3-((2-methoxy-4-(methylsulfonyl)phenyl)amino)prop-1- |
| | yn-1-yl)-3-(2,2,2-trifluoroethyl)benzo[b]thiophene 1-oxide; |
| 1528 | 4-((2-(3-((2-methoxy-4-(methylsulfonyl)phenyl)amino)prop-1-yn-1-yl)-1-oxido-3-(2,2,2- |
| | trifluoroethyl)benzo[b]thiophen-7-yl)amino)tetrahydro-2H-thiopyran 1,1-dioxide; |
| 1529 | 7-(((3R,4S)-3-fluoro-1-methylpiperidin-4-yl)amino)-2-(3-((2-methoxy-4- |
| | (methylsulfonyl)phenyl)amino)prop-1-yn-1-yl)-3-(2,2,2-trifluoroethyl)benzo[b]thiophene 1-oxide; |
| 1530 | 7-((3-fluoro-1-methylpiperidin-4-yl)amino)-2-(3-((2-methoxy-4-(methylsulfonyl)phenyl)amino)prop-1-yn- |
| | 1-yl)-3-(2,2,2-trifluoroethyl)benzo[b]thiophene 1-oxide; |
| 1531 | 2-(dimethylamino)-1-(4-((2-(3-((2-methoxy-4-(methylsulfonyl)phenyl)amino)prop-1-yn-1-yl)-1-oxido-3- |
| | (2,2,2-trifluoroethyl)benzo[b]thiophen-7-yl)amino)piperidin-1-yl)ethan-1-one; |
| 1532 | 7-((3-fluoro-1-(2-hydroxy-3-methoxypropyl)piperidin-4-yl)amino)-2-(3-((2-methoxy-4- |
| | (methylsulfonyl)phenyl)amino)prop-1-yn-1-yl)-3-(2,2,2-trifluoroethyl)benzo[b]thiophene 1-oxide; |
| 1533 | 7-(((3R,4R)-3-fluoro-1-methylpiperidin-4-yl)amino)-2-(3-((2-methoxy-4- |
| | (methylsulfonyl)phenyl)amino)prop-1-yn-1-yl)-3-(2,2,2-trifluoroethyl)benzo[b]thiophene 1-oxide; |
| 1534 | 7-(((3S,4S)-3-fluoro-1-methylpiperidin-4-yl)amino)-2-(3-((2-methoxy-4- |
| | (methylsulfonyl)phenyl)amino)prop-1-yn-1-yl)-3-(2,2,2-trifluoroethyl)benzo[b]thiophene 1-oxide; |
| 1535 | 7-((4-(dimethylamino)cyclohexyl)amino)-2-(3-((2-methoxy-4-(methylsulfonyl)phenyl)amino)prop-1-yn-1- |
| | yl)-3-(2,2,2-trifluoroethyl)benzo[b]thiophene 1-oxide; |
| 1536 | 7-((2-(dimethylamino)ethyl)amino)-2-(3-((2-methoxy-4-(methylsulfonyl)phenyl)amino)prop-1-yn-1-yl)-3- |
| | (2,2,2-trifluoroethyl)benzo[b]thiophene 1-oxide; |
| 1537 | 7-(((3S,4R)-3-fluoro-1-methylpiperidin-4-yl)amino)-2-(3-((2-methoxy-4- |
| | (methylsulfonyl)phenyl)amino)prop-1-yn-1-yl)-3-(2,2,2-trifluoroethyl)benzo[b]thiophene 1-oxide; |
| 1538 | 2-(3-((2-methoxy-4-(methylsulfonyl)phenyl)amino)prop-1-yn-1-yl)-7-((1-methylpiperidin-4-yl)amino)-3- |
| | (2,2,2-trifluoroethyl)benzo[b]thiophene 1-oxide; |
| 1539 | 2-(3-((2-methoxy-4-(methylsulfonyl)phenyl)amino)prop-1-yn-1-yl)-7-((tetrahydro-2H-pyran-4-yl)amino)- |
| | 3-(2,2,2-trifluoroethyl)benzo[b]thiophene 1-oxide; |
| 1540 | 3-(cyclopropylmethyl)-7-((3,3-difluoro-1-methylpiperidin-4-yl)amino)-2-(3-((2-methoxy-4- |
| | (methylsulfonyl)phenyl)amino)prop-1-yn-1-yl)benzo[b]thiophene 1-oxide; |
| 1541 | 4-((3-(cyclopropylmethyl)-2-(3-((2-methoxy-4-(methylsulfonyl)phenyl)amino)prop-1-yn-1-yl)-1- |
| | oxidobenzo[b]thiophen-7-yl)amino)tetrahydro-2H-thiopyran 1,1-dioxide; |
| 1542 | 3-(cyclopropylmethyl)-7-(((3R,4S)-3-fluoro-1-methylpiperidin-4-yl)amino)-2-(3-((2-methoxy-4- |
| | (methylsulfonyl)phenyl)amino)prop-1-yn-1-yl)benzo[b]thiophene 1-oxide; |
| 1543 | 3-(cyclopropylmethyl)-7-((3-fluoro-1-methylpiperidin-4-yl)amino)-2-(3-((2-methoxy-4- |
| | (methylsulfonyl)phenyl)amino)prop-1-yn-1-yl)benzo[b]thiophene 1-oxide; |
| 1544 | 1-(4-((3-(cyclopropylmethyl)-2-(3-((2-methoxy-4-(methylsulfonyl)phenyl)amino)prop-1-yn-1-yl)-1- |
| | oxidobenzo[b]thiophen-7-yl)amino)piperidin-1-yl)-2-(dimethylamino)ethan-1-one; |
| 1545 | 3-(cyclopropylmethyl)-7-((3-fluoro-1-(2-hydroxy-3-methoxypropyl)piperidin-4-yl)amino)-2-(3-((2- |
| | methoxy-4-(methylsulfonyl)phenyl)amino)prop-1-yn-1-yl)benzo[b]thiophene 1-oxide; |
| 1546 | 3-(cyclopropylmethyl)-7-(((3R,4R)-3-fluoro-1-methylpiperidin-4-yl)amino)-2-(3-((2-methoxy-4- |
| | (methylsulfonyl)phenyl)amino)prop-1-yn-1-yl)benzo[b]thiophene 1-oxide; |
| 1547 | 3-(cyclopropylmethyl)-7-(((3S,4S)-3-fluoro-1-methylpiperidin-4-yl)amino)-2-(3-((2-methoxy-4- |
| | (methylsulfonyl)phenyl)amino)prop-1-yn-1-yl)benzo[b]thiophene 1-oxide; |
| 1548 | 3-(cyclopropylmethyl)-7-((4-(dimethylamino)cyclohexyl)amino)-2-(3-((2-methoxy-4- |
| | (methylsulfonyl)phenyl)amino)prop-1-yn-1-yl)benzo[b]thiophene 1-oxide; |
| 1549 | 3-(cyclopropylmethyl)-7-((2-(dimethylamino)ethyl)amino)-2-(3-((2-methoxy-4- |
| | (methylsulfonyl)phenyl)amino)prop-1-yn-1-yl)benzo[b]thiophene 1-oxide; |
| 1550 | 3-(cyclopropylmethyl)-7-(((3S,4R)-3-fluoro-1-methylpiperidin-4-yl)amino)-2-(3-((2-methoxy-4- |
| | (methylsulfonyl)phenyl)amino)prop-1-yn-1-yl)benzo[b]thiophene 1-oxide; |
| 1551 | 3-(cyclopropylmethyl)-2-(3-((2-methoxy-4-(methylsulfonyl)phenyl)amino)prop-1-yn-1-yl)-7-((1- |
| | methylpiperidin-4-yl)amino)benzo[b]thiophene 1-oxide; |
| 1552 | 3-(cyclopropylmethyl)-2-(3-((2-methoxy-4-(methylsulfonyl)phenyl)amino)prop-1-yn-1-yl)-7-((tetrahydro- |
| | 2H-pyran-4-yl)amino)benzo[b]thiophene 1-oxide; |
| 1553 | 7-((3,3-difluoro-1-methylpiperidin-4-yl)amino)-3-(2,2-difluoroethyl)-2-(3-((2-methoxy-4- |
| | (methylsulfonyl)phenyl)amino)prop-1-yn-1-yl)benzo[b]thiophene 1-oxide; |
| 1554 | 4-((3-(2,2-difluoroethyl)-2-(3-((2-methoxy-4-(methylsulfonyl)phenyl)amino)prop-1-yn-1-yl)-1- |
| | oxidobenzo[b]thiophen-7-yl)amino)tetrahydro-2H-thiopyran 1,1-dioxide; |
| 1555 | 3-(2,2-difluoroethyl)-7-(((3R,4S)-3-fluoro-1-methylpiperidin-4-yl)amino)-2-(3-((2-methoxy-4- |
| | (methylsulfonyl)phenyl)amino)prop-1-yn-1-yl)benzo[b]thiophene 1-oxide; |
| 1556 | 3-(2,2-difluoroethyl)-7-((3-fluoro-1-methylpiperidin-4-yl)amino)-2-(3-((2-methoxy-4- |
| | (methylsulfonyl)phenyl)amino)prop-1-yn-1-yl)benzo[b]thiophene 1-oxide; |
| 1557 | 1-(4-((3-(2,2-difluoroethyl)-2-(3-((2-methoxy-4-(methylsulfonyl)phenyl)amino)prop-1-yn-1-yl)-1- |
| | oxidobenzo[b]thiophen-7-yl)amino)piperidin-1-yl)-2-(dimethylamino)ethan-1-one; |
| 1558 | 3-(2,2-difluoroethyl)-7-((3-fluoro-1-(2-hydroxy-3-methoxypropyl)piperidin-4-yl)amino)-2-(3-((2-methoxy- |
| | 4-(methylsulfonyl)phenyl)amino)prop-1-yn-1-yl)benzo[b]thiophene 1-oxide; |
| 1559 | 3-(2,2-difluoroethyl)-7-(((3R,4R)-3-fluoro-1-methylpiperidin-4-yl)amino)-2-(3-((2-methoxy-4- |
| | (methylsulfonyl)phenyl)amino)prop-1-yn-1-yl)benzo[b]thiophene 1-oxide; |
| 1560 | 3-(2,2-difluoroethyl)-7-(((3S,4S)-3-fluoro-1-methylpiperidin-4-yl)amino)-2-(3-((2-methoxy-4- |
| | (methylsulfonyl)phenyl)amino)prop-1-yn-1-yl)benzo[b]thiophene 1-oxide; |
| 1561 | 3-(2,2-difluoroethyl)-7-((4-(dimethylamino)cyclohexyl)amino)-2-(3-((2-methoxy-4- |
| | (methylsulfonyl)phenyl)amino)prop-1-yn-1-yl)benzo[b]thiophene 1-oxide; |
| 1562 | 3-(2,2-difluoroethyl)-7-((2-(dimethylamino)ethyl)amino)-2-(3-((2-methoxy-4- |
| | (methylsulfonyl)phenyl)amino)prop-1-yn-1-yl)benzo[b]thiophene 1-oxide; |
| 1563 | 3-(2,2-difluoroethyl)-7-(((3S,4R)-3-fluoro-1-methylpiperidin-4-yl)amino)-2-(3-((2-methoxy-4- |
| | (methylsulfonyl)phenyl)amino)prop-1-yn-1-yl)benzo[b]thiophene 1-oxide; |
| 1564 | 3-(2,2-difluoroethyl)-2-(3-((2-methoxy-4-(methylsulfonyl)phenyl)amino)prop-1-yn-1-yl)-7-((1- |
| | methylpiperidin-4-yl)amino)benzo[b]thiophene 1-oxide; |
| 1565 | 3-(2,2-difluoroethyl)-2-(3-((2-methoxy-4-(methylsulfonyl)phenyl)amino)prop-1-yn-1-yl)-7-((tetrahydro- |
| | 2H-pyran-4-yl)amino)benzo[b]thiophene 1-oxide; |
| 1566 | 7-((3,3-difluoro-1-methylpiperidin-4-yl)amino)-2-(3-((2-methoxy-4-(methylsulfonyl)phenyl)amino)prop-1- |
| | yn-1-yl)-3-(1H-pyrrol-1-yl)benzo[b]thiophene 1-oxide; |
| 1567 | 4-((2-(3-((2-methoxy-4-(methylsulfonyl)phenyl)amino)prop-1-yn-1-yl)-1-oxido-3-(1H-pyrrol-1- |
| | yl)benzo[b]thiophen-7-yl)amino)tetrahydro-2H-thiopyran 1,1-dioxide; |
| 1568 | 7-(((3R,4S)-3-fluoro-1-methylpiperidin-4-yl)amino)-2-(3-((2-methoxy-4- |
| | (methylsulfonyl)phenyl)amino)prop-1-yn-1-yl)-3-(1H-pyrrol-1-yl)benzo[b]thiophene 1-oxide; |
| 1569 | 7-((3-fluoro-1-methylpiperidin-4-yl)amino)-2-(3-((2-methoxy-4-(methylsulfonyl)phenyl)amino)prop-1-yn- |
| | 1-yl)-3-(1H-pyrrol-1-yl)benzo[b]thiophene 1-oxide; |
| 1570 | 2-(dimethylamino)-1-(4-((2-(3-((2-methoxy-4-(methylsulfonyl)phenyl)amino)prop-1-yn-1-yl)-1-oxido-3- |
| | (1H-pyrrol-1-yl)benzo[b]thiophen-7-yl)amino)piperidin-1-yl)ethan-1-one; |
| 1571 | 7-((3-fluoro-1-(2-hydroxy-3-methoxypropyl)piperidin-4-yl)amino)-2-(3-((2-methoxy-4- |
| | (methylsulfonyl)phenyl)amino)prop-1-yn-1-yl)-3-(1H-pyrrol-1-yl)benzo[b]thiophene 1-oxide; |
| 1572 | 7-(((3R,4R)-3-fluoro-1-methylpiperidin-4-yl)amino)-2-(3-((2-methoxy-4- |
| | (methylsulfonyl)phenyl)amino)prop-1-yn-1-yl)-3-(1H-pyrrol-1-yl)benzo[b]thiophene 1-oxide; |
| 1573 | 7-(((3S,4S)-3-fluoro-1-methylpiperidin-4-yl)amino)-2-(3-((2-methoxy-4- |
| | (methylsulfonyl)phenyl)amino)prop-1-yn-1-yl)-3-(1H-pyrrol-1-yl)benzo[b]thiophene 1-oxide; |
| 1574 | 7-((4-(dimethylamino)cyclohexyl)amino)-2-(3-((2-methoxy-4-(methylsulfonyl)phenyl)amino)prop-1-yn-1- |
| | yl)-3-(1H-pyrrol-1-yl)benzo[b]thiophene 1-oxide; |
| 1575 | 7-((2-(dimethylamino)ethyl)amino)-2-(3-((2-methoxy-4-(methylsulfonyl)phenyl)amino)prop-1-yn-1-yl)-3- |
| | (1H-pyrrol-1-yl)benzo[b]thiophene 1-oxide; |
| 1576 | 7-(((3S,4R)-3-fluoro-1-methylpiperidin-4-yl)amino)-2-(3-((2-methoxy-4- |
| | (methylsulfonyl)phenyl)amino)prop-1-yn-1-yl)-3-(1H-pyrrol-1-yl)benzo[b]thiophene 1-oxide; |
| 1577 | 2-(3-((2-methoxy-4-(methylsulfonyl)phenyl)amino)prop-1-yn-1-yl)-7-((1-methylpiperidin-4-yl)amino)-3- |
| | (1H-pyrrol-1-yl)benzo[b]thiophene 1-oxide; |
| 1578 | 2-(3-((2-methoxy-4-(methylsulfonyl)phenyl)amino)prop-1-yn-1-yl)-3-(1H-pyrrol-1-yl)-7-((tetrahydro-2H- |
| | pyran-4-yl)amino)benzo[b]thiophene 1-oxide; |
| 1579 | 7-((3,3-difluoro-1-methylpiperidin-4-yl)amino)-3-ethyl-2-(3-((2-methoxy-4- |
| | (methylsulfonyl)phenyl)amino)prop-1-yn-1-yl)benzo[b]thiophene 1-oxide; |
| 1580 | 4-((3-ethyl-2-(3-((2-methoxy-4-(methylsulfonyl)phenyl)amino)prop-1-yn-1-yl)-1-oxidobenzo[b]thiophen-7- |
| | yl)amino)tetrahydro-2H-thiopyran 1,1-dioxide; |
| 1581 | 3-ethyl-7-(((3R,4S)-3-fluoro-1-methylpiperidin-4-yl)amino)-2-(3-((2-methoxy-4- |
| | (methylsulfonyl)phenyl)amino)prop-1-yn-1-yl)benzo[b]thiophene 1-oxide; |
| 1582 | 3-ethyl-7-((3-fluoro-1-methylpiperidin-4-yl)amino)-2-(3-((2-methoxy-4- |
| | (methylsulfonyl)phenyl)amino)prop-1-yn-1-yl)benzo[b]thiophene 1-oxide; |
| 1583 | 2-(dimethylamino)-1-(4-((3-ethyl-2-(3-((2-methoxy-4-(methylsulfonyl)phenyl)amino)prop-1-yn-1-yl)-1- |
| | oxidobenzo[b]thiophen-7-yl)amino)piperidin-1-yl)ethan-1-one; |
| 1584 | 3-ethyl-7-((3-fluoro-1-(2-hydroxy-3-methoxypropyl)piperidin-4-yl)amino)-2-(3-((2-methoxy-4- |
| | (methylsulfonyl)phenyl)amino)prop-1-yn-1-yl)benzo[b]thiophene 1-oxide; |
| 1585 | 3-ethyl-7-(((3R,4R)-3-fluoro-1-methylpiperidin-4-yl)amino)-2-(3-((2-methoxy-4- |
| | (methylsulfonyl)phenyl)amino)prop-1-yn-1-yl)benzo[b]thiophene 1-oxide; |
| 1586 | 3-ethyl-7-(((3S,4S)-3-fluoro-1-methylpiperidin-4-yl)amino)-2-(3-((2-methoxy-4- |
| | (methylsulfonyl)phenyl)amino)prop-1-yn-1-yl)benzo[b]thiophene 1-oxide; |
| 1587 | 7-((4-(dimethylamino)cyclohexyl)amino)-3-ethyl-2-(3-((2-methoxy-4-(methylsulfonyl)phenyl)amino)prop- |
| | 1-yn-1-yl)benzo[b]thiophene 1-oxide; |
| 1588 | 7-((2-(dimethylamino)ethyl)amino)-3-ethyl-2-(3-((2-methoxy-4-(methylsulfonyl)phenyl)amino)prop-1-yn- |
| | 1-yl)benzo[b]thiophene 1-oxide; |
| 1589 | 3-ethyl-7-(((3S,4R)-3-fluoro-1-methylpiperidin-4-yl)amino)-2-(3-((2-methoxy-4- |
| | (methylsulfonyl)phenyl)amino)prop-1-yn-1-yl)benzo[b]thiophene 1-oxide; |
| 1590 | 3-ethyl-2-(3-((2-methoxy-4-(methylsulfonyl)phenyl)amino)prop-1-yn-1-yl)-7-((1-methylpiperidin-4- |
| | yl)amino)benzo[b]thiophene 1-oxide; |
| 1591 | 3-ethyl-2-(3-((2-methoxy-4-(methylsulfonyl)phenyl)amino)prop-1-yn-1-yl)-7-((tetrahydro-2H-pyran-4- |
| | yl)amino)benzo[b]thiophene 1-oxide; |
| 1592 | 4-((3-(3-(cyclopropylmethyl)-7-((3,3-difluoro-1-methylpiperidin-4-yl)amino)thieno[3,2-c]pyridin-2- |
| | yl)prop-2-yn-1-yl)amino)benzenesulfonamide; |
| 1593 | 4-((3-(3-(cyclopropylmethyl)-7-((3-fluoro-1-(2-hydroxy-3-methoxypropyl)piperidin-4-yl)amino)thieno[3,2- |
| | c]pyridin-2-yl)prop-2-yn-1-yl)amino)benzenesulfonamide; |
| 1594 | 4-((3-(3-(cyclopropylmethyl)-7-((3-fluoro-1-methylpiperidin-4-yl)amino)thieno[3,2-c]pyridin-2-yl)prop-2- |
| | yn-1-yl)amino)benzenesulfonamide; |
| 1595 | 4-((3-(3-(cyclopropylmethyl)-7-((4-(dimethylamino)cyclohexyl)amino)thieno[3,2-c]pyridin-2-yl)prop-2-yn- |
| | 1-yl)amino)benzenesulfonamide; |
| 1596 | 4-((3-(3-(cyclopropylmethyl)-7-((1-methylpiperidin-4-yl)amino)thieno[3,2-c]pyridin-2-yl)prop-2-yn-1- |
| | yl)amino)benzenesulfonamide; |
| 1597 | 4-((3-(7-((3,3-difluoro-1-methylpiperidin-4-yl)amino)-3-ethylthieno[3,2-c]pyridin-2-yl)prop-2-yn-1- |
| | yl)amino)benzenesulfonamide; |
| 1598 | 4-((3-(3-ethyl-7-((3-fluoro-1-(2-hydroxy-3-methoxypropyl)piperidin-4-yl)amino)thieno[3,2-c]pyridin-2- |
| | yl)prop-2-yn-1-yl)amino)benzenesulfonamide; |
| 1599 | 4-((3-(3-ethyl-7-((3-fluoro-1-methylpiperidin-4-yl)amino)thieno[3,2-c]pyridin-2-yl)prop-2-yn-1- |
| | yl)amino)benzenesulfonamide; |
| 1600 | 4-((3-(7-((4-(dimethylamino)cyclohexyl)amino)-3-ethylthieno[3,2-c]pyridin-2-yl)prop-2-yn-1- |
| | yl)amino)benzenesulfonamide; |
| 1601 | 4-((3-(3-ethyl-7-((1-methylpiperidin-4-yl)amino)thieno[3,2-c]pyridin-2-yl)prop-2-yn-1- |
| | yl)amino)benzenesulfonamide; |
| 1602 | 4-((3-(7-((3,3-difluoro-1-methylpiperidin-4-yl)amino)-3-(2,2,2-trifluoroethyl)thieno[3,2-c]pyridin-2- |
| | yl)prop-2-yn-1-yl)amino)benzenesulfonamide; |
| 1603 | 4-((3-(7-((3-fluoro-1-(2-hydroxy-3-methoxypropyl)piperidin-4-yl)amino)-3-(2,2,2-trifluoroethyl)thieno[3,2- |
| | c]pyridin-2-yl)prop-2-yn-1-yl)amino)benzenesulfonamide; |
| 1604 | 4-((3-(7-((3-fluoro-1-methylpiperidin-4-yl)amino)-3-(2,2,2-trifluoroethyl)thieno[3,2-c]pyridin-2-yl)prop-2- |
| | yn-1-yl)amino)benzenesulfonamide; |
| 1605 | 4-((3-(7-((4-(dimethylamino)cyclohexyl)amino)-3-(2,2,2-trifluoroethyl)thieno[3,2-c]pyridin-2-yl)prop-2- |
| | yn-1-yl)amino)benzenesulfonamide; |
| 1606 | 4-((3-(7-((1-methylpiperidin-4-yl)amino)-3-(2,2,2-trifluoroethyl)thieno[3,2-c]pyridin-2-yl)prop-2-yn-1- |
| | yl)amino)benzenesulfonamide; |
| 1607 | 4-((3-(3-(cyclopropylmethyl)-7-((3,3-difluoro-1-methylpiperidin-4-yl)amino)thieno[3,2-b]pyridin-2- |
| | yl)prop-2-yn-1-yl)amino)benzenesulfonamide; |
| 1608 | 4-((3-(7-((3,3-difluoro-1-methylpiperidin-4-yl)amino)-3-ethylthieno[3,2-b]pyridin-2-yl)prop-2-yn-1- |
| | yl)amino)benzenesulfonamide; |
| 1609 | 4-((3-(7-((3,3-difluoro-1-methylpiperidin-4-yl)amino)-3-(2,2,2-trifluoroethyl)thieno[3,2-b]pyridin-2- |
| | yl)prop-2-yn-1-yl)amino)benzenesulfonamide; |
| 1610 | 4-((3-(3-(cyclopropylmethyl)-7-((3-fluoro-1-(2-hydroxy-3-methoxypropyl)piperidin-4-yl)amino)thieno[3,2- |
| | b]pyridin-2-yl)prop-2-yn-1-yl)amino)benzenesulfonamide; |
| 1611 | 4-((3-(3-ethyl-7-((3-fluoro-1-(2-hydroxy-3-methoxypropyl)piperidin-4-yl)amino)thieno[3,2-b]pyridin-2- |
| | yl)prop-2-yn-1-yl)amino)benzenesulfonamide; |
| 1612 | 4-((3-(7-((3-fluoro-1-(2-hydroxy-3-methoxypropyl)piperidin-4-yl)amino)-3-(2,2,2-trifluoroethyl)thieno[3,2- |
| | b]pyridin-2-yl)prop-2-yn-1-yl)amino)benzenesulfonamide; |
| 1613 | 4-((3-(3-(cyclopropylmethyl)-7-((3-fluoro-1-methylpiperidin-4-yl)amino)thieno[3,2-b]pyridin-2-yl)prop-2- |
| | yn-1-yl)amino)benzenesulfonamide; |
| 1614 | 4-((3-(3-ethyl-7-((3-fluoro-1-methylpiperidin-4-yl)amino)thieno[3,2-b]pyridin-2-yl)prop-2-yn-1- |
| | yl)amino)benzenesulfonamide; |
| 1615 | 4-((3-(7-((3-fluoro-1-methylpiperidin-4-yl)amino)-3-(2,2,2-trifluoroethyl)thieno[3,2-b]pyridin-2-yl)prop-2- |
| | yn-1-yl)amino)benzenesulfonamide; |
| 1616 | 4-((3-(3-(cyclopropylmethyl)-7-((4-(dimethylamino)cyclohexyl)amino)thieno[3,2-b]pyridin-2-yl)prop-2- |
| | yn-1-yl)amino)benzenesulfonamide; |
| 1617 | 4-((3-(7-((4-(dimethylamino)cyclohexyl)amino)-3-ethylthieno[3,2-b]pyridin-2-yl)prop-2-yn-1- |
| | yl)amino)benzenesulfonamide; |
| 1618 | 4-((3-(7-((4-(dimethylamino)cyclohexyl)amino)-3-(2,2,2-trifluoroethyl)thieno[3,2-b]pyridin-2-yl)prop-2- |
| | yn-1-yl)amino)benzenesulfonamide; |
| 1619 | 4-((3-(3-(cyclopropylmethyl)-7-((1-methylpiperidin-4-yl)amino)thieno[3,2-b]pyridin-2-yl)prop-2-yn-1- |
| | yl)amino)benzenesulfonamide; |
| 1620 | 4-((3-(3-ethyl-7-((1-methylpiperidin-4-yl)amino)thieno[3,2-b]pyridin-2-yl)prop-2-yn-1- |
| | yl)amino)benzenesulfonamide; |
| 1621 | 4-((3-(7-((1-methylpiperidin-4-yl)amino)-3-(2,2,2-trifluoroethyl)thieno[3,2-b]pyridin-2-yl)prop-2-yn-1- |
| | yl)amino)benzenesulfonamide; |
| 1622 | 4-((3-(3-(cyclopropylmethyl)-7-((3,3-difluoro-1-methylpiperidin-4-yl)amino)thieno[2,3-c]pyridin-2- |
| | yl)prop-2-yn-1-yl)amino)benzenesulfonamide; |
| 1623 | 4-((3-(3-(cyclopropylmethyl)-7-((3-fluoro-1-(2-hydroxy-3-methoxypropyl)piperidin-4-yl)amino)thieno[2,3- |
| | c]pyridin-2-yl)prop-2-yn-1-yl)amino)benzenesulfonamide; |
| 1624 | 4-((3-(3-(cyclopropylmethyl)-7-((3-fluoro-1-methylpiperidin-4-yl)amino)thieno[2,3-c]pyridin-2-yl)prop-2- |
| | yn-1-yl)amino)benzenesulfonamide; |
| 1625 | 4-((3-(3-(cyclopropylmethyl)-7-((4-(dimethylamino)cyclohexyl)amino)thieno[2,3-c]pyridin-2-yl)prop-2-yn- |
| | 1-yl)amino)benzenesulfonamide; |
| 1626 | 4-((3-(3-(cyclopropylmethyl)-7-((1-methylpiperidin-4-yl)amino)thieno[2,3-c]pyridin-2-yl)prop-2-yn-1- |
| | yl)amino)benzenesulfonamide; |
| 1627 | 4-((3-(7-((3,3-difluoro-1-methylpiperidin-4-yl)amino)-3-ethylthieno[2,3-c]pyridin-2-yl)prop-2-yn-1- |
| | yl)amino)benzenesulfonamide; |
| 1628 | 4-((3-(3-ethyl-7-((3-fluoro-1-(2-hydroxy-3-methoxypropyl)piperidin-4-yl)amino)thieno[2,3-c]pyridin-2- |
| | yl)prop-2-yn-1-yl)amino)benzenesulfonamide; |
| 1629 | 4-((3-(3-ethyl-7-((3-fluoro-1-methylpiperidin-4-yl)amino)thieno[2,3-c]pyridin-2-yl)prop-2-yn-1- |
| | yl)amino)benzenesulfonamide; |
| 1630 | 4-((3-(7-((4-(dimethylamino)cyclohexyl)amino)-3-ethylthieno[2,3-c]pyridin-2-yl)prop-2-yn-1- |
| | yl)amino)benzenesulfonamide; |
| 1631 | 4-((3-(3-ethyl-7-((1-methylpiperidin-4-yl)amino)thieno[2,3-c]pyridin-2-yl)prop-2-yn-1- |
| | yl)amino)benzenesulfonamide; |
| 1632 | 4-((3-(7-((3,3-difluoro-1-methylpiperidin-4-yl)amino)-3-(2,2,2-trifluoroethyl)thieno[2,3-c]pyridin-2- |
| | yl)prop-2-yn-1-yl)amino)benzenesulfonamide; |
| 1633 | 4-((3-(7-((3-fluoro-1-(2-hydroxy-3-methoxypropyl)piperidin-4-yl)amino)-3-(2,2,2-trifluoroethyl)thieno[2,3- |
| | c]pyridin-2-yl)prop-2-yn-1-yl)amino)benzenesulfonamide; |
| 1634 | 4-((3-(7-((3-fluoro-1-methylpiperidin-4-yl)amino)-3-(2,2,2-trifluoroethyl)thieno[2,3-c]pyridin-2-yl)prop-2- |
| | yn-1-yl)amino)benzenesulfonamide; |
| 1635 | 4-((3-(7-((4-(dimethylamino)cyclohexyl)amino)-3-(2,2,2-trifluoroethyl)thieno[2,3-c]pyridin-2-yl)prop-2- |
| | yn-1-yl)amino)benzenesulfonamide; |
| 1636 | 4-((3-(7-((1-methylpiperidin-4-yl)amino)-3-(2,2,2-trifluoroethyl)thieno[2,3-c]pyridin-2-yl)prop-2-yn-1- |
| | yl)amino)benzenesulfonamide; |
| 1637 | 4-((3-(3-(cyclopropylmethyl)-7-((4-(dimethylamino)cyclohexyl)amino)benzo[b]thiophen-2-yl)prop-2-yn-1- |
| | yl)amino)benzenesulfonamide; |
| 1638 | 4-((3-(3-(cyclopropylmethyl)-7-((3,3-difluoro-1-methylpiperidin-4-yl)amino)benzo[b]thiophen-2-yl)prop-2- |
| | yn-1-yl)amino)benzenesulfonamide; |
| 1639 | 4-((3-(3-(cyclopropylmethyl)-7-((1-methylpiperidin-4-yl)amino)benzo[b]thiophen-2-yl)prop-2-yn-1- |
| | yl)amino)benzenesulfonamide; |
| 1640 | 4-((3-(3-(cyclopropylmethyl)-7-((3-fluoro-1-methylpiperidin-4-yl)amino)benzo[b]thiophen-2-yl)prop-2-yn- |
| | 1-yl)amino)benzenesulfonamide; |
| 1641 | 4-((3-(3-(cyclopropylmethyl)-7-((3-fluoro-1-(2-hydroxy-3-methoxypropyl)piperidin-4- |
| | yl)amino)benzo[b]thiophen-2-yl)prop-2-yn-1-yl)amino)benzenesulfonamide; |
| 1642 | 4-((3-(7-((4-(dimethylamino)cyclohexyl)amino)-3-ethylbenzo[b]thiophen-2-yl)prop-2-yn-1- |
| | yl)amino)benzenesulfonamide; |
| 1643 | 4-((3-(7-((3,3-difluoro-1-methylpiperidin-4-yl)amino)-3-ethylbenzo[b]thiophen-2-yl)prop-2-yn-1- |
| | yl)amino)benzenesulfonamide; |
| 1644 | 4-((3-(3-ethyl-7-((1-methylpiperidin-4-yl)amino)benzo[b]thiophen-2-yl)prop-2-yn-1- |
| | yl)amino)benzenesulfonamide; |
| 1645 | 4-((3-(3-ethyl-7-((3-fluoro-1-methylpiperidin-4-yl)amino)benzo[b]thiophen-2-yl)prop-2-yn-1- |
| | yl)amino)benzenesulfonamide; |
| 1646 | 4-((3-(3-ethyl-7-((3-fluoro-1-(2-hydroxy-3-methoxypropyl)piperidin-4-yl)amino)benzo[b]thiophen-2- |
| | yl)prop-2-yn-1-yl)amino)benzenesulfonamide; |
| 1647 | 4-((3-(3-(cyclopropylmethyl)-7-((4-(dimethylamino)cyclohexyl)amino)benzofuran-2-yl)prop-2-yn-1- |
| | yl)amino)benzenesulfonamide; |
| 1648 | 4-((3-(7-((4-(dimethylamino)cyclohexyl)amino)-3-ethylbenzofuran-2-yl)prop-2-yn-1- |
| | yl)amino)benzenesulfonamide; |
| 1649 | 4-((3-(3-(cyclopropylmethyl)-7-((3,3-difluoro-1-methylpiperidin-4-yl)amino)benzofuran-2-yl)prop-2-yn-1- |
| | yl)amino)benzenesulfonamide; |
| 1650 | 4-((3-(7-((3,3-difluoro-1-methylpiperidin-4-yl)amino)-3-ethylbenzofuran-2-yl)prop-2-yn-1- |
| | yl)amino)benzenesulfonamide; |
| 1651 | 4-((3-(3-(cyclopropylmethyl)-7-((1-methylpiperidin-4-yl)amino)benzofuran-2-yl)prop-2-yn-1- |
| | yl)amino)benzenesulfonamide; |
| 1652 | 4-((3-(3-ethyl-7-((1-methylpiperidin-4-yl)amino)benzofuran-2-yl)prop-2-yn-1- |
| | yl)amino)benzenesulfonamide; |
| 1653 | 4-((3-(3-(cyclopropylmethyl)-7-((3-fluoro-1-methylpiperidin-4-yl)amino)benzofuran-2-yl)prop-2-yn-1- |
| | yl)amino)benzenesulfonamide; |
| 1654 | 4-((3-(3-ethyl-7-((3-fluoro-1-methylpiperidin-4-yl)amino)benzofuran-2-yl)prop-2-yn-1- |
| | yl)amino)benzenesulfonamide; |
| 1655 | 4-((3-(3-(cyclopropylmethyl)-7-((3-fluoro-1-(2-hydroxy-3-methoxypropyl)piperidin-4- |
| | yl)amino)benzofuran-2-yl)prop-2-yn-1-yl)amino)benzenesulfonamide; |
| 1656 | 4-((3-(3-ethyl-7-((3-fluoro-1-(2-hydroxy-3-methoxypropyl)piperidin-4-yl)amino)benzofuran-2-yl)prop-2- |
| | yn-1-yl)amino)benzenesulfonamide; |
| 1657 | 4-((3-(3-(cyclopropylmethyl)-7-((4-(dimethylamino)cyclohexyl)amino)-1-oxidobenzo[b]thiophen-2- |
| | yl)prop-2-yn-1-yl)amino)benzenesulfonamide; |
| 1658 | 4-((3-(3-(cyclopropylmethyl)-7-((3,3-difluoro-1-methylpiperidin-4-yl)amino)-1-oxidobenzo[b]thiophen-2- |
| | yl)prop-2-yn-1-yl)amino)benzenesulfonamide; |
| 1659 | 4-((3-(3-(cyclopropylmethyl)-7-((1-methylpiperidin-4-yl)amino)-1-oxidobenzo[b]thiophen-2-yl)prop-2-yn- |
| | 1-yl)amino)benzenesulfonamide; |
| 1660 | 4-((3-(3-(cyclopropylmethyl)-7-((3-fluoro-1-methylpiperidin-4-yl)amino)-1-oxidobenzo[b]thiophen-2- |
| | yl)prop-2-yn-1-yl)amino)benzenesulfonamide; |
| 1661 | 4-((3-(3-(cyclopropylmethyl)-7-((3-fluoro-1-(2-hydroxy-3-methoxypropyl)piperidin-4-yl)amino)-1- |
| | oxidobenzo[b]thiophen-2-yl)prop-2-yn-1-yl)amino)benzenesulfonamide; |
| 1662 | 4-((3-(7-((4-(dimethylamino)cyclohexyl)amino)-3-ethyl-1-oxidobenzo[b]thiophen-2-yl)prop-2-yn-1- |
| | yl)amino)benzenesulfonamide; |
| 1663 | 4-((3-(7-((3,3-difluoro-1-methylpiperidin-4-yl)amino)-3-ethyl-1-oxidobenzo[b]thiophen-2-yl)prop-2-yn-1- |
| | yl)amino)benzenesulfonamide; |
| 1664 | 4-((3-(3-ethyl-7-((1-methylpiperidin-4-yl)amino)-1-oxidobenzo[b]thiophen-2-yl)prop-2-yn-1- |
| | yl)amino)benzenesulfonamide; |
| 1665 | 4-((3-(3-ethyl-7-((3-fluoro-1-methylpiperidin-4-yl)amino)-1-oxidobenzo[b]thiophen-2-yl)prop-2-yn-1- |
| | yl)amino)benzenesulfonamide; |
| 1666 | 4-((3-(3-ethyl-7-((3-fluoro-1-(2-hydroxy-3-methoxypropyl)piperidin-4-yl)amino)-1- |
| | oxidobenzo[b]thiophen-2-yl)prop-2-yn-1-yl)amino)benzenesulfonamide; |
| 1667 | 4-((3-(3-(cyclopropylmethyl)-7-((4-(dimethylamino)cyclohexyl)amino)-1,1-dioxidobenzo[b]thiophen-2- |
| | yl)prop-2-yn-1-yl)amino)benzenesulfonamide; |
| 1668 | 4-((3-(3-(cyclopropylmethyl)-7-((3,3-difluoro-1-methylpiperidin-4-yl)amino)-1,1-dioxidobenzo[b]thiophen- |
| | 2-yl)prop-2-yn-1-yl)amino)benzenesulfonamide; |
| 1669 | 4-((3-(3-(cyclopropylmethyl)-7-((1-methylpiperidin-4-yl)amino)-1,1-dioxidobenzo[b]thiophen-2-yl)prop-2- |
| | yn-1-yl)amino)benzenesulfonamide; |
| 1670 | 4-((3-(3-(cyclopropylmethyl)-7-((3-fluoro-1-methylpiperidin-4-yl)amino)-1,1-dioxidobenzo[b]thiophen-2- |
| | yl)prop-2-yn-1-yl)amino)benzenesulfonamide; |
| 1671 | 4-((3-(3-(cyclopropylmethyl)-7-((3-fluoro-1-(2-hydroxy-3-methoxypropyl)piperidin-4-yl)amino)-1,1- |
| | dioxidobenzo[b]thiophen-2-yl)prop-2-yn-1-yl)amino)benzenesulfonamide; |
| 1672 | 4-((3-(7-((4-(dimethylamino)cyclohexyl)amino)-3-ethyl-1,1-dioxidobenzo[b]thiophen-2-yl)prop-2-yn-1- |
| | yl)amino)benzenesulfonamide; |
| 1673 | 4-((3-(7-((3,3-difluoro-1-methylpiperidin-4-yl)amino)-3-ethyl-1,1-dioxidobenzo[b]thiophen-2-yl)prop-2- |
| | yn-1-yl)amino)benzenesulfonamide; |
| 1674 | 4-((3-(3-ethyl-7-((1-methylpiperidin-4-yl)amino)-1,1-dioxidobenzo[b]thiophen-2-yl)prop-2-yn-1- |
| | yl)amino)benzenesulfonamide; |
| 1675 | 4-((3-(3-ethyl-7-((3-fluoro-1-methylpiperidin-4-yl)amino)-1,1-dioxidobenzo[b]thiophen-2-yl)prop-2-yn-1- |
| | yl)amino)benzenesulfonamide; |
| 1676 | 4-((3-(3-ethyl-7-((3-fluoro-1-(2-hydroxy-3-methoxypropyl)piperidin-4-yl)amino)-1,1- |
| | dioxidobenzo[b]thiophen-2-yl)prop-2-yn-1-yl)amino)benzenesulfonamide; |
| 1677 | 4-((3-(7-((4-(dimethylamino)cyclohexyl)amino)-3-(2,2,2-trifluoroethyl)benzo[b]thiophen-2-yl)prop-2-yn-1- |
| | yl)amino)benzenesulfonamide; |
| 1678 | 4-((3-(7-((3,3-difluoro-1-methylpiperidin-4-yl)amino)-3-(2,2,2-trifluoroethyl)benzo[b]thiophen-2-yl)prop- |
| | 2-yn-1-yl)amino)benzenesulfonamide; |
| 1679 | 4-((3-(7-((1-methylpiperidin-4-yl)amino)-3-(2,2,2-trifluoroethyl)benzo[b]thiophen-2-yl)prop-2-yn-1- |
| | yl)amino)benzenesulfonamide; |
| 1680 | 4-((3-(7-((3-fluoro-1-methylpiperidin-4-yl)amino)-3-(2,2,2-trifluoroethyl)benzo[b]thiophen-2-yl)prop-2-yn- |
| | 1-yl)amino)benzenesulfonamide; |
| 1681 | 4-((3-(7-((3-fluoro-1-(2-hydroxy-3-methoxypropyl)piperidin-4-yl)amino)-3-(2,2,2- |
| | trifluoroethyl)benzo[b]thiophen-2-yl)prop-2-yn-1-yl)amino)benzenesulfonamide; |
| 1682 | 4-((3-(7-((4-(dimethylamino)cyclohexyl)amino)-3-(2,2,2-trifluoroethyl)benzofuran-2-yl)prop-2-yn-1- |
| | yl)amino)benzenesulfonamide; |
| 1683 | 4-((3-(7-((3,3-difluoro-1-methylpiperidin-4-yl)amino)-3-(2,2,2-trifluoroethyl)benzofuran-2-yl)prop-2-yn-1- |
| | yl)amino)benzenesulfonamide; |
| 1684 | 4-((3-(7-((1-methylpiperidin-4-yl)amino)-3-(2,2,2-trifluoroethyl)benzofuran-2-yl)prop-2-yn-1- |
| | yl)amino)benzenesulfonamide; |
| 1685 | 4-((3-(7-((3-fluoro-1-methylpiperidin-4-yl)amino)-3-(2,2,2-trifluoroethyl)benzofuran-2-yl)prop-2-yn-1- |
| | yl)amino)benzenesulfonamide; |
| 1686 | 4-((3-(7-((3-fluoro-1-(2-hydroxy-3-methoxypropyl)piperidin-4-yl)amino)-3-(2,2,2- |
| | trifluoroethyl)benzofuran-2-yl)prop-2-yn-1-yl)amino)benzenesulfonamide; |
| 1687 | 4-((3-(7-((4-(dimethylamino)cyclohexyl)amino)-1-oxido-3-(2,2,2-trifluoroethyl)benzo[b]thiophen-2- |
| | yl)prop-2-yn-1-yl)amino)benzenesulfonamide; |
| 1688 | 4-((3-(7-((3,3-difluoro-1-methylpiperidin-4-yl)amino)-1-oxido-3-(2,2,2-trifluoroethyl)benzo[b]thiophen-2- |
| | yl)prop-2-yn-1-yl)amino)benzenesulfonamide; |
| 1689 | 4-((3-(7-((1-methylpiperidin-4-yl)amino)-1-oxido-3-(2,2,2-trifluoroethyl)benzo[b]thiophen-2-yl)prop-2-yn- |
| | 1-yl)amino)benzenesulfonamide; |
| 1690 | 4-((3-(7-((3-fluoro-1-methylpiperidin-4-yl)amino)-1-oxido-3-(2,2,2-trifluoroethyl)benzo[b]thiophen-2- |
| | yl)prop-2-yn-1-yl)amino)benzenesulfonamide; |
| 1691 | 4-((3-(7-((3-fluoro-1-(2-hydroxy-3-methoxypropyl)piperidin-4-yl)amino)-1-oxido-3-(2,2,2- |
| | trifluoroethyl)benzo[b]thiophen-2-yl)prop-2-yn-1-yl)amino)benzenesulfonamide; |
| 1692 | 4-((3-(7-((4-(dimethylamino)cyclohexyl)amino)-1,1-dioxido-3-(2,2,2-trifluoroethyl)benzo[b]thiophen-2- |
| | yl)prop-2-yn-1-yl)amino)benzenesulfonamide; |
| 1693 | 4-((3-(7-((3,3-difluoro-1-methylpiperidin-4-yl)amino)-1,1-dioxido-3-(2,2,2- |
| | trifluoroethyl)benzo[b]thiophen-2-yl)prop-2-yn-1-yl)amino)benzenesulfonamide; |
| 1694 | 4-((3-(7-((1-methylpiperidin-4-yl)amino)-1,1-dioxido-3-(2,2,2-trifluoroethyl)benzo[b]thiophen-2-yl)prop-2- |
| | yn-1-yl)amino)benzenesulfonamide; |
| 1695 | 4-((3-(7-((3-fluoro-1-methylpiperidin-4-yl)amino)-1,1-dioxido-3-(2,2,2-trifluoroethyl)benzo[b]thiophen-2- |
| | yl)prop-2-yn-1-yl)amino)benzenesulfonamide; or |
| 1696 | 4-((3-(7-((3-fluoro-1-(2-hydroxy-3-methoxypropyl)piperidin-4-yl)amino)-1,1-dioxido-3-(2,2,2- |
| | trifluoroethyl)benzo[b]thiophen-2-yl)prop-2-yn-1-yl)amino)benzenesulfonamide; |

or the compound of formula (I) is selected from 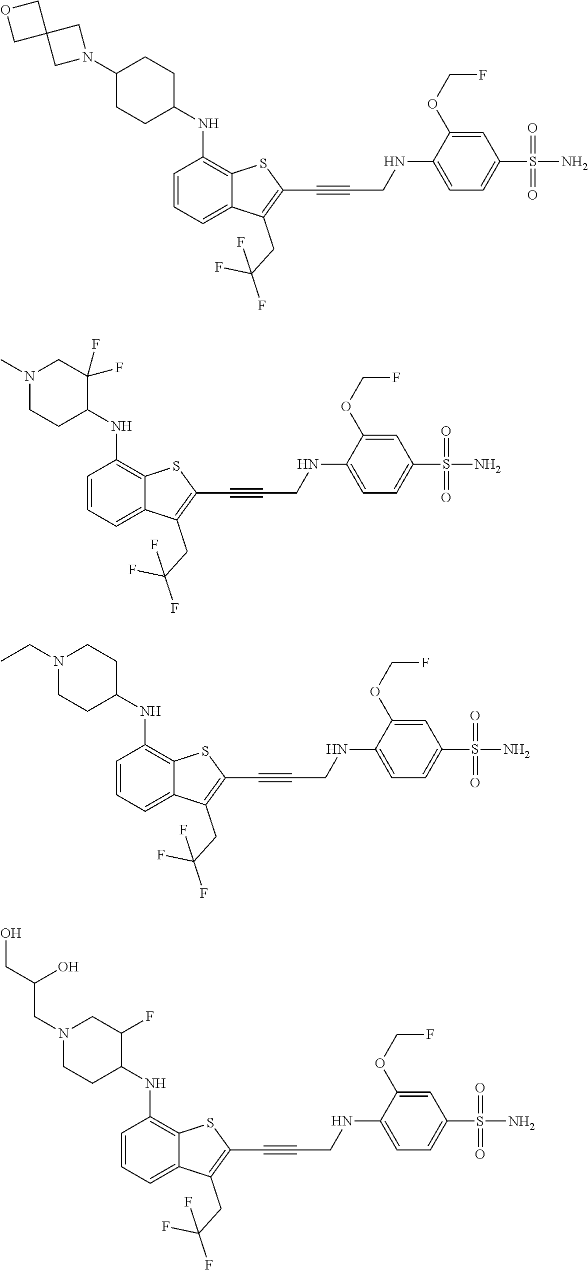 [SMILES: NS(=O)(=O)c1ccc(NCC#Cc2sc3c(NC4CCC(N5CC6(COC6)C5)CC4)cccc3c2CC(F)(F)F)c(OCF)c1 ; CN1CCC(Nc2cccc3c(CC(F)(F)F)c(C#CCNc4ccc(S(N)(=O)=O)cc4OCF)sc23)C(F)(F)C1 ; CCN1CCC(Nc2cccc3c(CC(F)(F)F)c(C#CCNc4ccc(S(N)(=O)=O)cc4OCF)sc23)CC1 ; NS(=O)(=O)c1ccc(NCC#Cc2sc3c(NC4CCN(CC(O)CO)CC4F)cccc3c2CC(F)(F)F)c(OCF)c1] 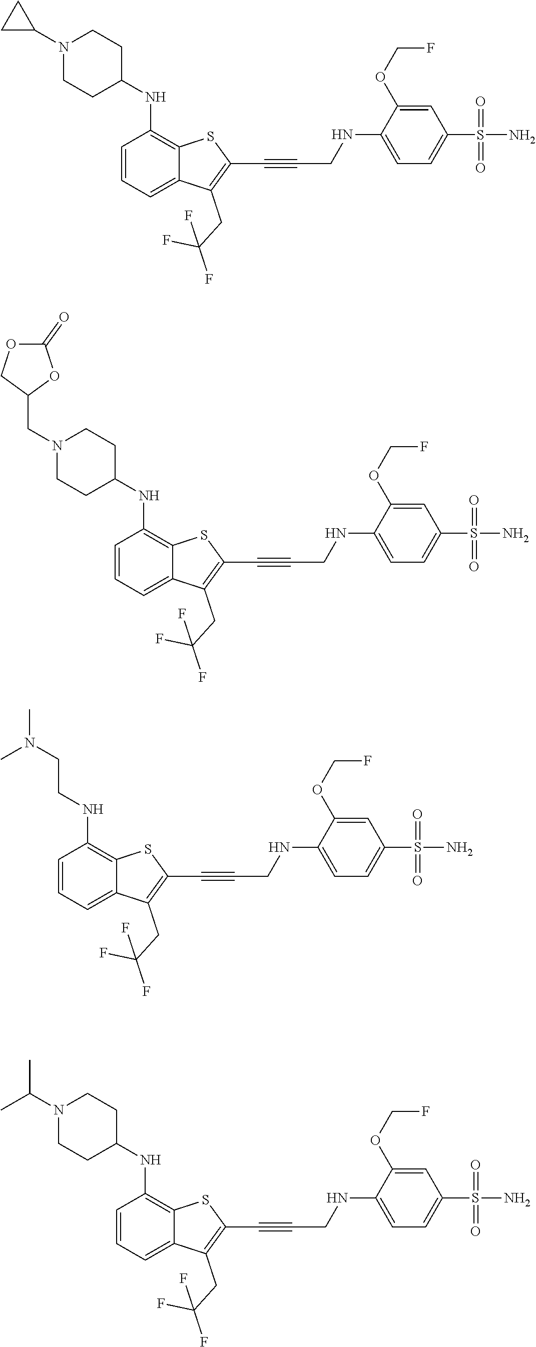 [SMILES: NS(=O)(=O)c1ccc(NCC#Cc2sc3c(NC4CCN(C5CC5)CC4)cccc3c2CC(F)(F)F)c(OCF)c1 ; NS(=O)(=O)c1ccc(NCC#Cc2sc3c(NC4CCN(CC5COC(=O)O5)CC4)cccc3c2CC(F)(F)F)c(OCF)c1 ; CN(C)CCNc1cccc2c(CC(F)(F)F)c(C#CCNc3ccc(S(N)(=O)=O)cc3OCF)sc12 ; CC(C)N1CCC(Nc2cccc3c(CC(F)(F)F)c(C#CCNc4ccc(S(N)(=O)=O)cc4OCF)sc23)CC1] 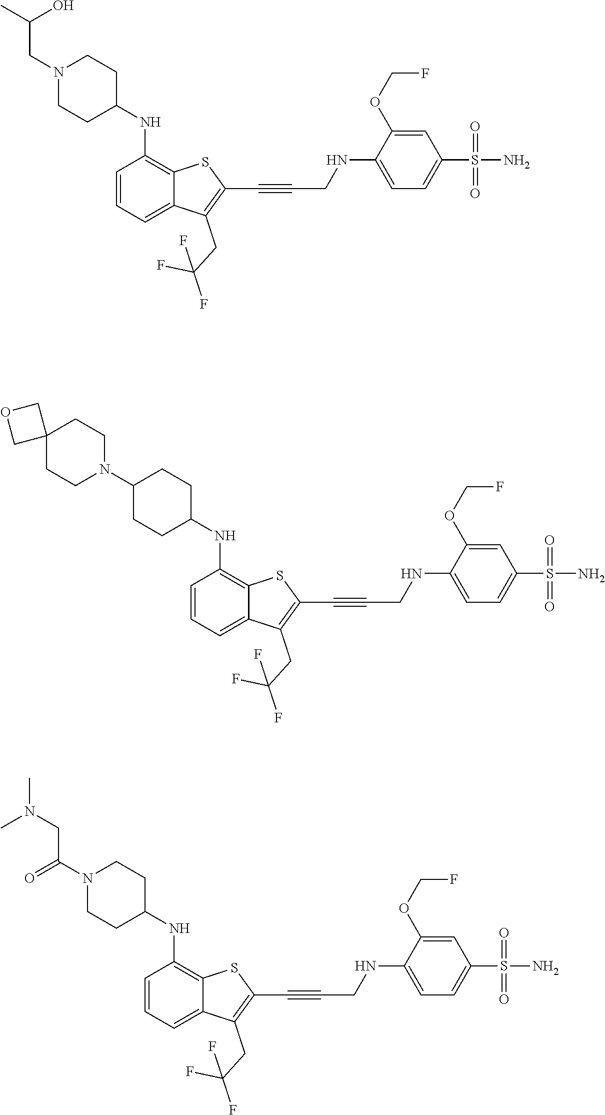 [SMILES: CC(O)CN1CCC(Nc2cccc3c(CC(F)(F)F)c(C#CCNc4ccc(S(N)(=O)=O)cc4OCF)sc23)CC1 ; NS(=O)(=O)c1ccc(NCC#Cc2sc3c(NC4CCC(N5CCC6(CC5)COC6)CC4)cccc3c2CC(F)(F)F)c(OCF)c1 ; CN(C)CC(=O)N1CCC(Nc2cccc3c(CC(F)(F)F)c(C#CCNc4ccc(S(N)(=O)=O)cc4OCF)sc23)CC1] 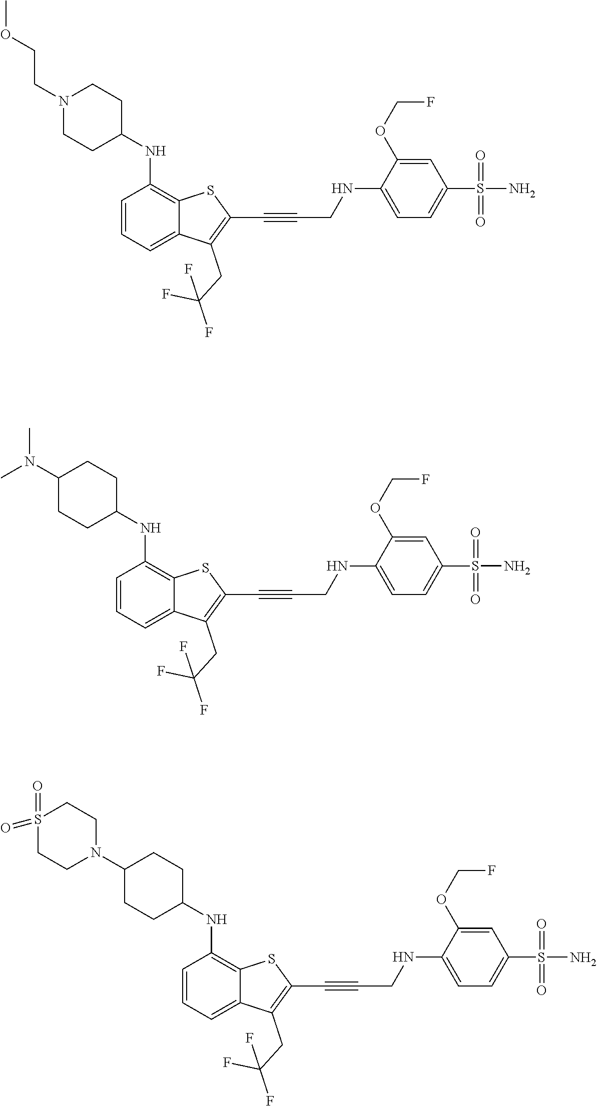 [SMILES: COCCN1CCC(Nc2cccc3c(CC(F)(F)F)c(C#CCNc4ccc(S(N)(=O)=O)cc4OCF)sc23)CC1 ; CN(C)C1CCC(Nc2cccc3c(CC(F)(F)F)c(C#CCNc4ccc(S(N)(=O)=O)cc4OCF)sc23)CC1 ; NS(=O)(=O)c1ccc(NCC#Cc2sc3c(NC4CCC(N5CCS(=O)(=O)CC5)CC4)cccc3c2CC(F)(F)F)c(OCF)c1] 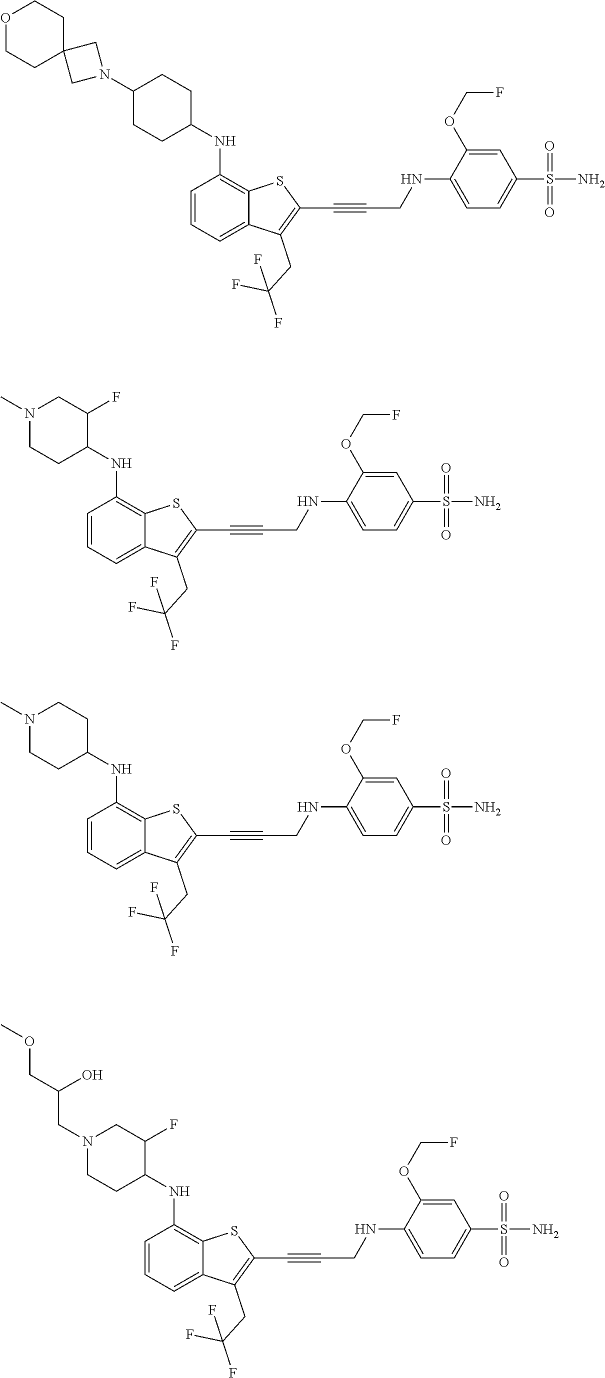 [SMILES: NS(=O)(=O)c1ccc(NCC#Cc2sc3c(NC4CCC(N5CC6(CCOCC6)C5)CC4)cccc3c2CC(F)(F)F)c(OCF)c1 ; CN1CCC(Nc2cccc3c(CC(F)(F)F)c(C#CCNc4ccc(S(N)(=O)=O)cc4OCF)sc23)C(F)C1 ; CN1CCC(Nc2cccc3c(CC(F)(F)F)c(C#CCNc4ccc(S(N)(=O)=O)cc4OCF)sc23)CC1 ; COCC(O)CN1CCC(Nc2cccc3c(CC(F)(F)F)c(C#CCNc4ccc(S(N)(=O)=O)cc4OCF)sc23)C(F)C1] 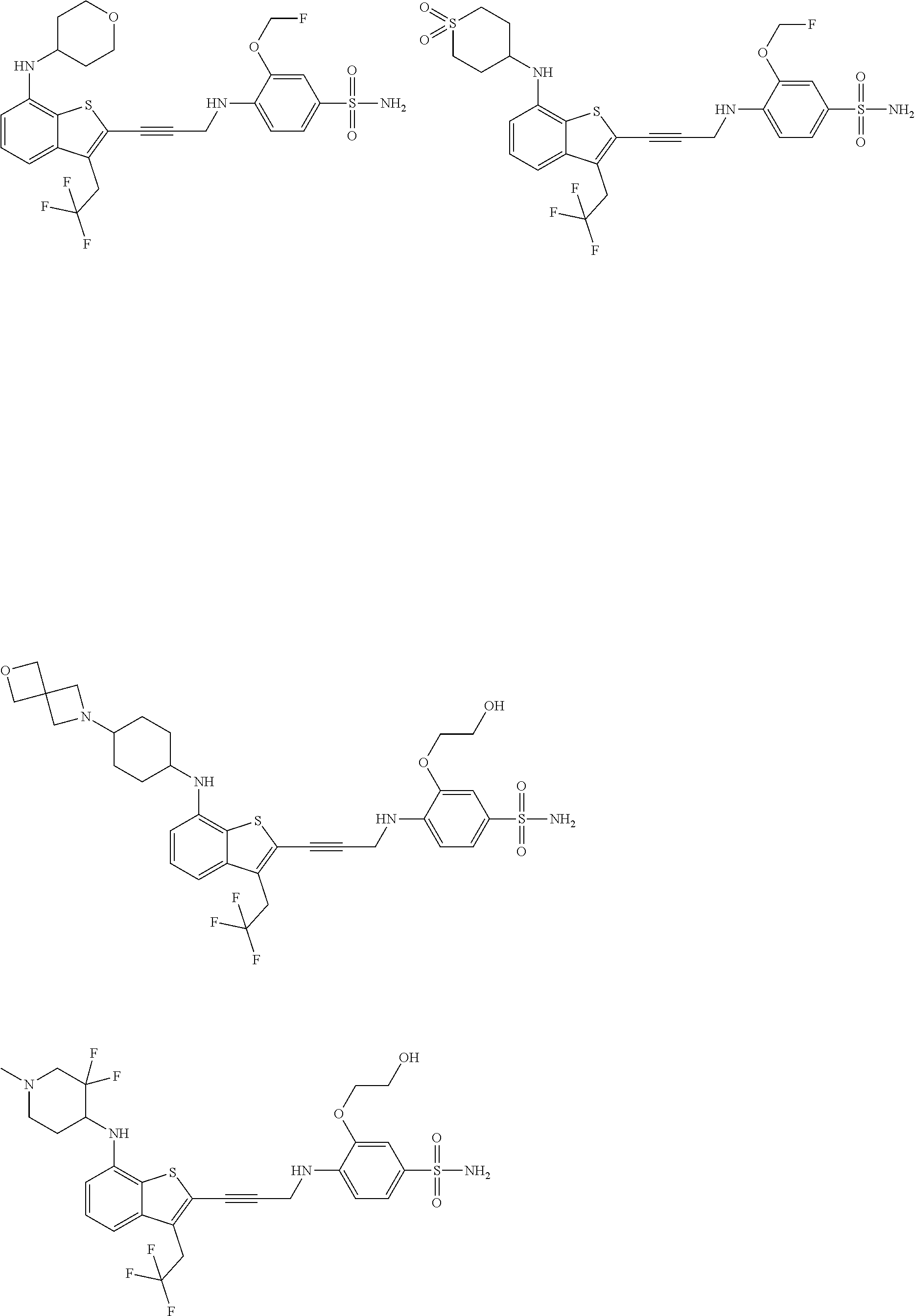 [SMILES: NS(=O)(=O)c1ccc(NCC#Cc2sc3c(NC4CCOCC4)cccc3c2CC(F)(F)F)c(OCF)c1 ; NS(=O)(=O)c1ccc(NCC#Cc2sc3c(NC4CCS(=O)(=O)CC4)cccc3c2CC(F)(F)F)c(OCF)c1 ; NS(=O)(=O)c1ccc(NCC#Cc2sc3c(NC4CCC(N5CC6(COC6)C5)CC4)cccc3c2CC(F)(F)F)c(OCCO)c1 ; CN1CCC(Nc2cccc3c(CC(F)(F)F)c(C#CCNc4ccc(S(N)(=O)=O)cc4OCCO)sc23)C(F)(F)C1] 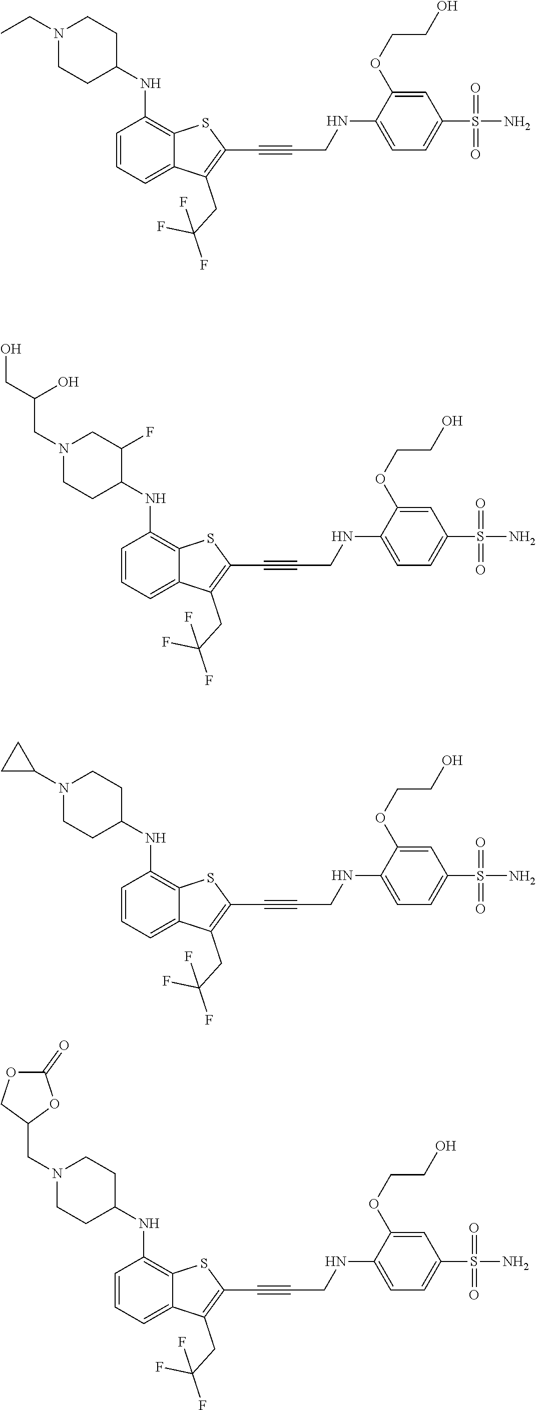 [SMILES: CCN1CCC(Nc2cccc3c(CC(F)(F)F)c(C#CCNc4ccc(S(N)(=O)=O)cc4OCCO)sc23)CC1 ; NS(=O)(=O)c1ccc(NCC#Cc2sc3c(NC4CCN(CC(O)CO)CC4F)cccc3c2CC(F)(F)F)c(OCCO)c1 ; NS(=O)(=O)c1ccc(NCC#Cc2sc3c(NC4CCN(C5CC5)CC4)cccc3c2CC(F)(F)F)c(OCCO)c1 ; NS(=O)(=O)c1ccc(NCC#Cc2sc3c(NC4CCN(CC5COC(=O)O5)CC4)cccc3c2CC(F)(F)F)c(OCCO)c1] 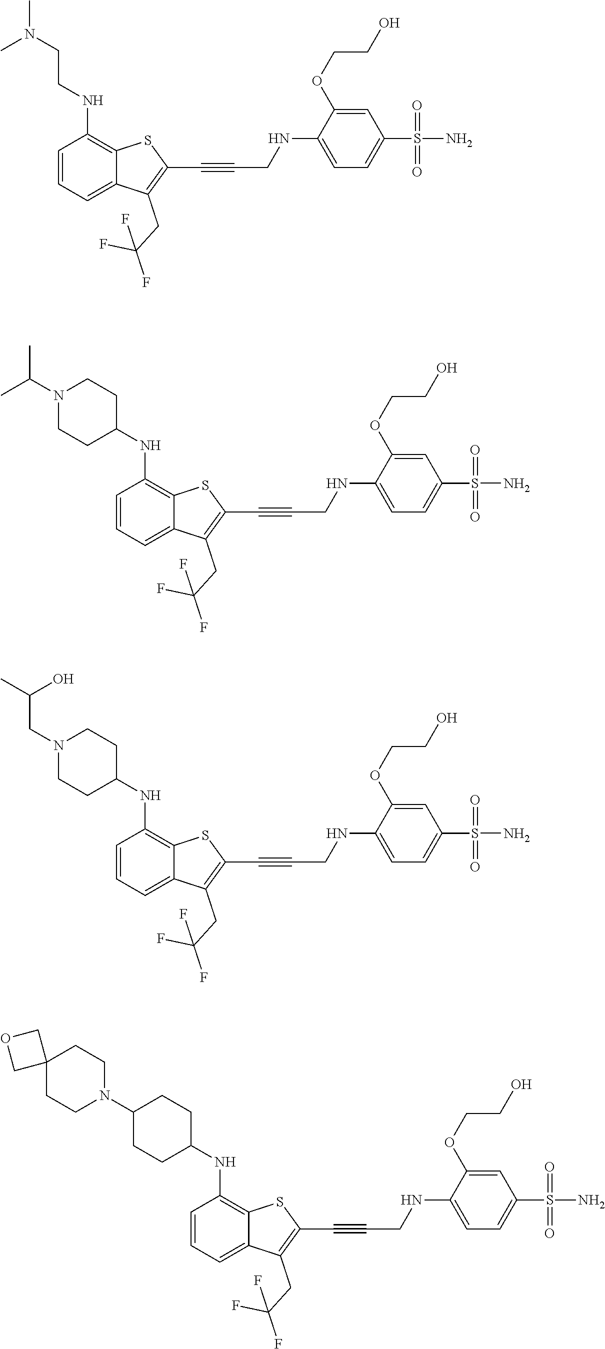 [SMILES: CN(C)CCNc1cccc2c(CC(F)(F)F)c(C#CCNc3ccc(S(N)(=O)=O)cc3OCCO)sc12 ; CC(C)N1CCC(Nc2cccc3c(CC(F)(F)F)c(C#CCNc4ccc(S(N)(=O)=O)cc4OCCO)sc23)CC1 ; CC(O)CN1CCC(Nc2cccc3c(CC(F)(F)F)c(C#CCNc4ccc(S(N)(=O)=O)cc4OCCO)sc23)CC1 ; NS(=O)(=O)c1ccc(NCC#Cc2sc3c(NC4CCC(N5CCC6(CC5)COC6)CC4)cccc3c2CC(F)(F)F)c(OCCO)c1] 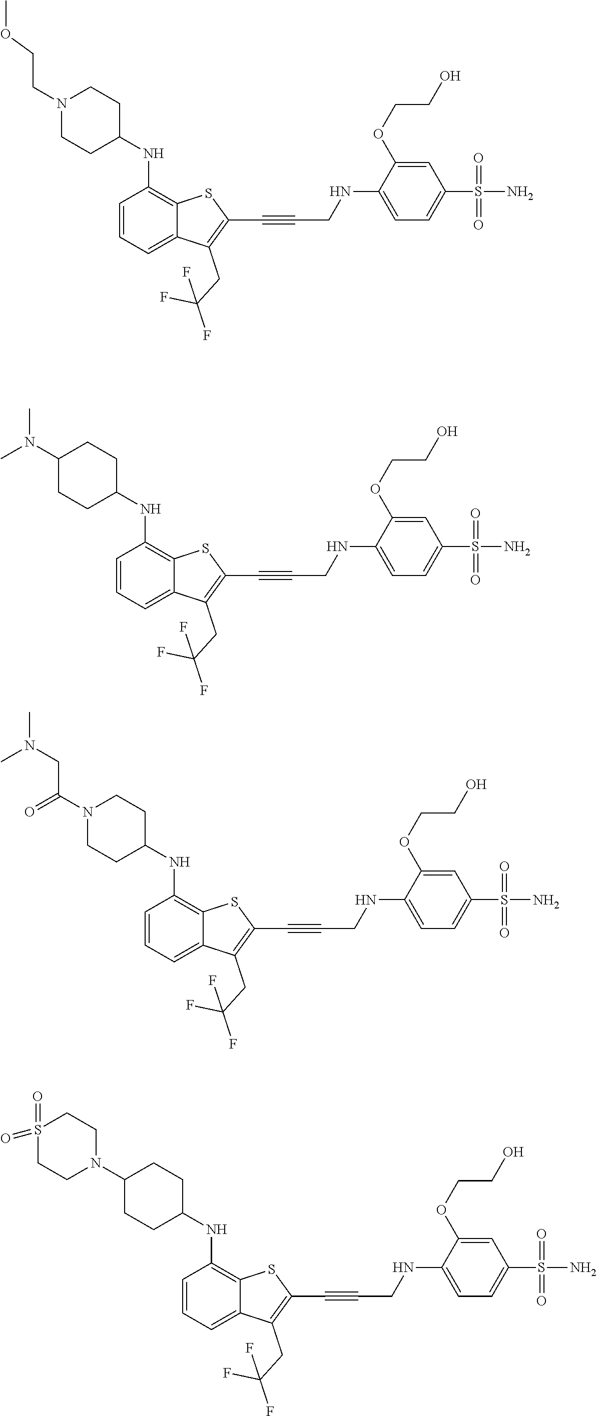 [SMILES: COCCN1CCC(Nc2cccc3c(CC(F)(F)F)c(C#CCNc4ccc(S(N)(=O)=O)cc4OCCO)sc23)CC1 ; CN(C)C1CCC(Nc2cccc3c(CC(F)(F)F)c(C#CCNc4ccc(S(N)(=O)=O)cc4OCCO)sc23)CC1 ; CN(C)CC(=O)N1CCC(Nc2cccc3c(CC(F)(F)F)c(C#CCNc4ccc(S(N)(=O)=O)cc4OCCO)sc23)CC1 ; NS(=O)(=O)c1ccc(NCC#Cc2sc3c(NC4CCC(N5CCS(=O)(=O)CC5)CC4)cccc3c2CC(F)(F)F)c(OCCO)c1] 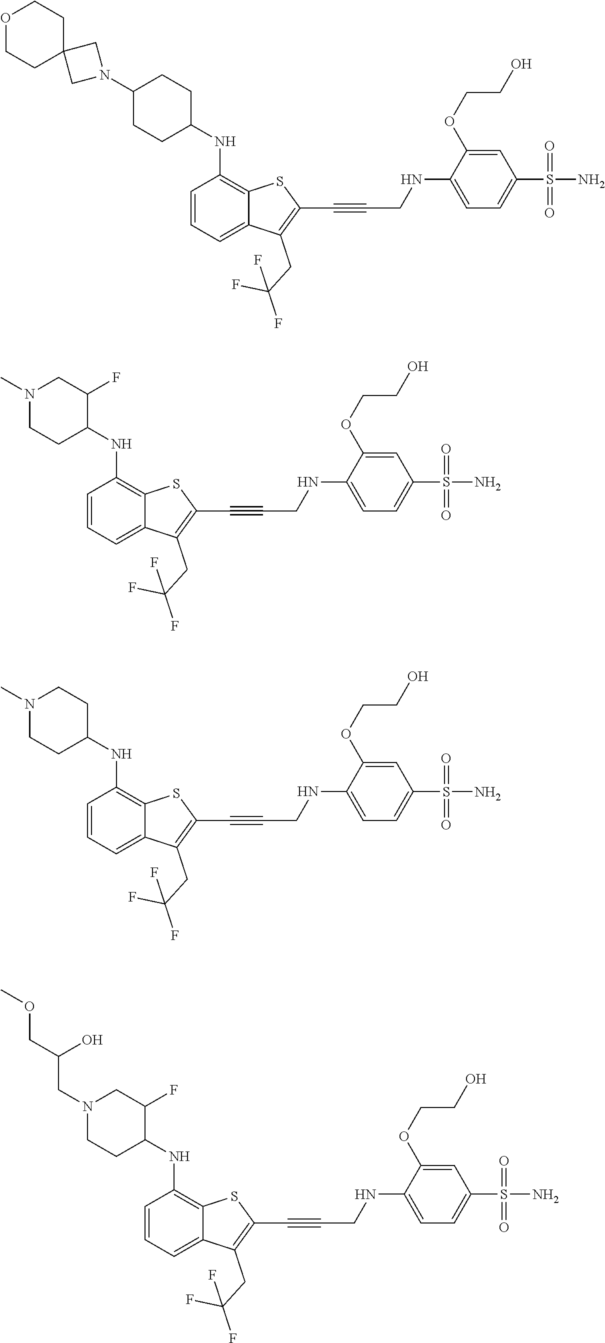 [SMILES: NS(=O)(=O)c1ccc(NCC#Cc2sc3c(NC4CCC(N5CC6(CCOCC6)C5)CC4)cccc3c2CC(F)(F)F)c(OCCO)c1 ; CN1CCC(Nc2cccc3c(CC(F)(F)F)c(C#CCNc4ccc(S(N)(=O)=O)cc4OCCO)sc23)C(F)C1 ; CN1CCC(Nc2cccc3c(CC(F)(F)F)c(C#CCNc4ccc(S(N)(=O)=O)cc4OCCO)sc23)CC1 ; COCC(O)CN1CCC(Nc2cccc3c(CC(F)(F)F)c(C#CCNc4ccc(S(N)(=O)=O)cc4OCCO)sc23)C(F)C1] 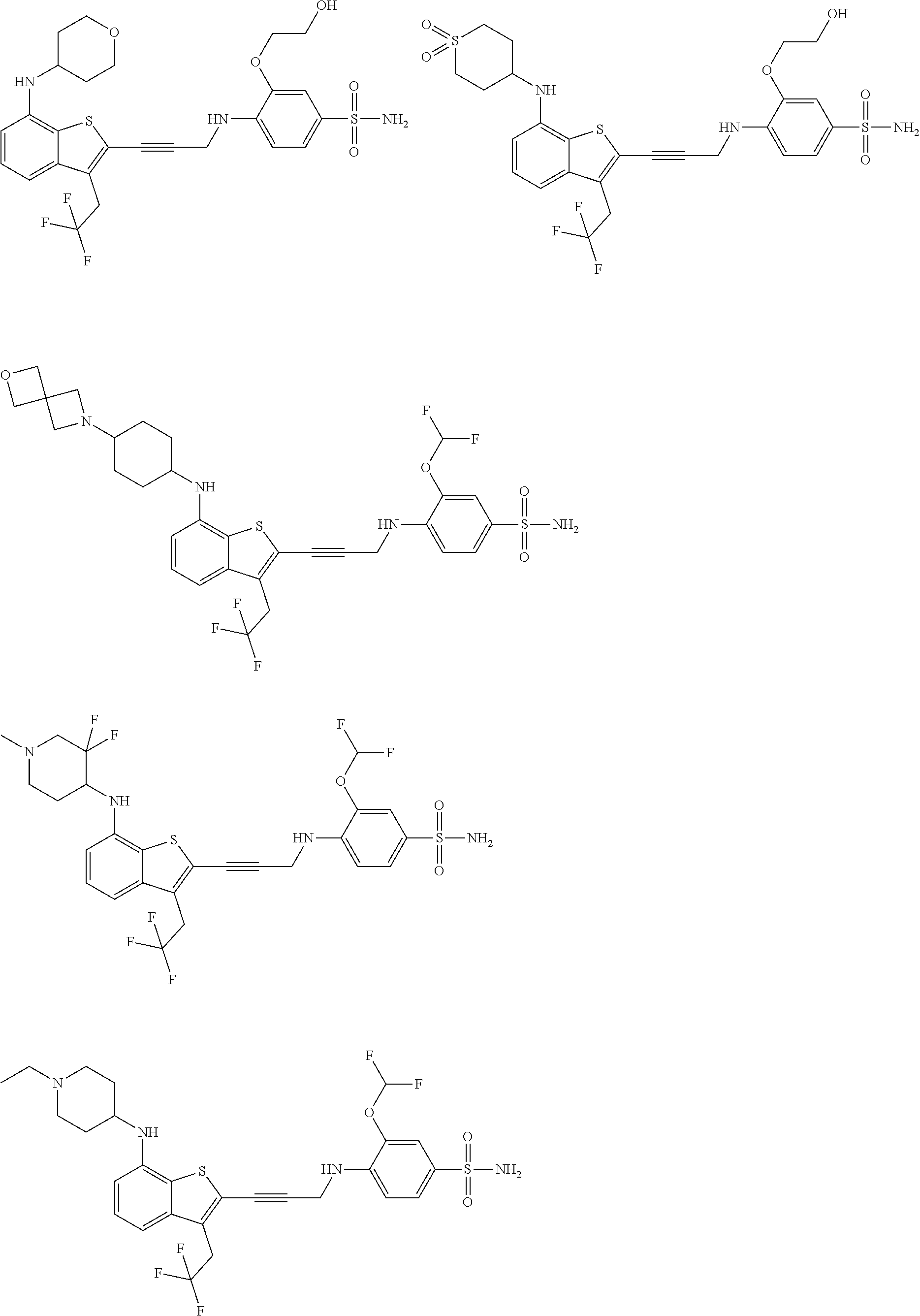 [SMILES: NS(=O)(=O)c1ccc(NCC#Cc2sc3c(NC4CCOCC4)cccc3c2CC(F)(F)F)c(OCCO)c1 ; NS(=O)(=O)c1ccc(NCC#Cc2sc3c(NC4CCS(=O)(=O)CC4)cccc3c2CC(F)(F)F)c(OCCO)c1 ; NS(=O)(=O)c1ccc(NCC#Cc2sc3c(NC4CCC(N5CC6(COC6)C5)CC4)cccc3c2CC(F)(F)F)c(OC(F)F)c1 ; CN1CCC(Nc2cccc3c(CC(F)(F)F)c(C#CCNc4ccc(S(N)(=O)=O)cc4OC(F)F)sc23)C(F)(F)C1 ; CCN1CCC(Nc2cccc3c(CC(F)(F)F)c(C#CCNc4ccc(S(N)(=O)=O)cc4OC(F)F)sc23)CC1] 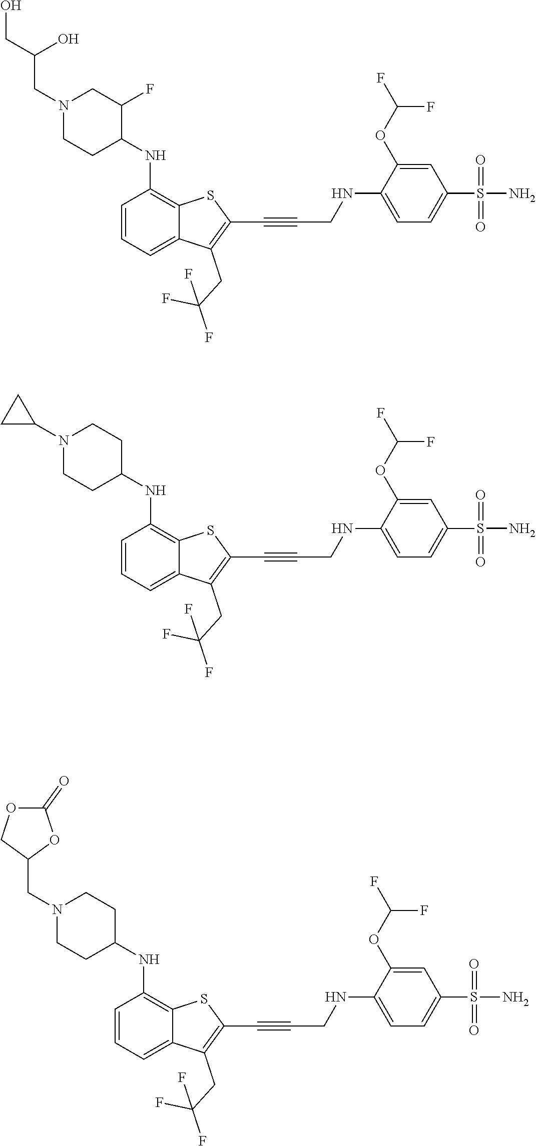 [SMILES: NS(=O)(=O)c1ccc(NCC#Cc2sc3c(NC4CCN(CC(O)CO)CC4F)cccc3c2CC(F)(F)F)c(OC(F)F)c1 ; NS(=O)(=O)c1ccc(NCC#Cc2sc3c(NC4CCN(C5CC5)CC4)cccc3c2CC(F)(F)F)c(OC(F)F)c1 ; NS(=O)(=O)c1ccc(NCC#Cc2sc3c(NC4CCN(CC5COC(=O)O5)CC4)cccc3c2CC(F)(F)F)c(OC(F)F)c1] 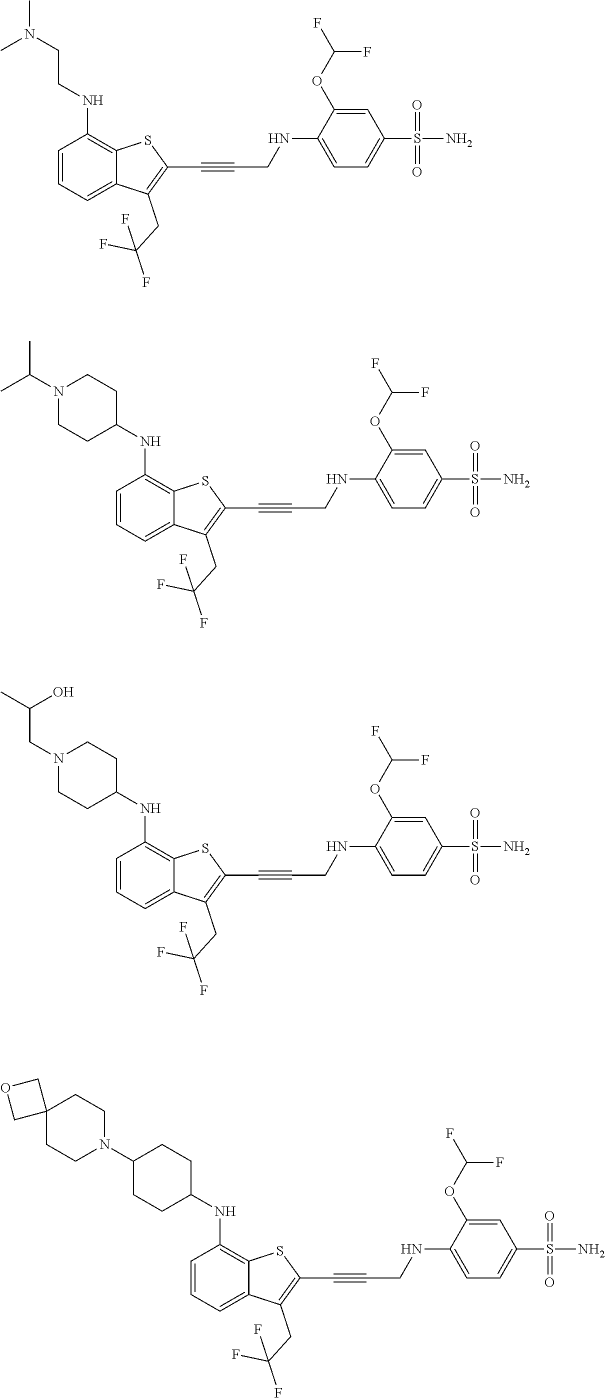 [SMILES: CN(C)CCNc1cccc2c(CC(F)(F)F)c(C#CCNc3ccc(S(N)(=O)=O)cc3OC(F)F)sc12 ; CC(C)N1CCC(Nc2cccc3c(CC(F)(F)F)c(C#CCNc4ccc(S(N)(=O)=O)cc4OC(F)F)sc23)CC1 ; CC(O)CN1CCC(Nc2cccc3c(CC(F)(F)F)c(C#CCNc4ccc(S(N)(=O)=O)cc4OC(F)F)sc23)CC1 ; NS(=O)(=O)c1ccc(NCC#Cc2sc3c(NC4CCC(N5CCC6(CC5)COC6)CC4)cccc3c2CC(F)(F)F)c(OC(F)F)c1] 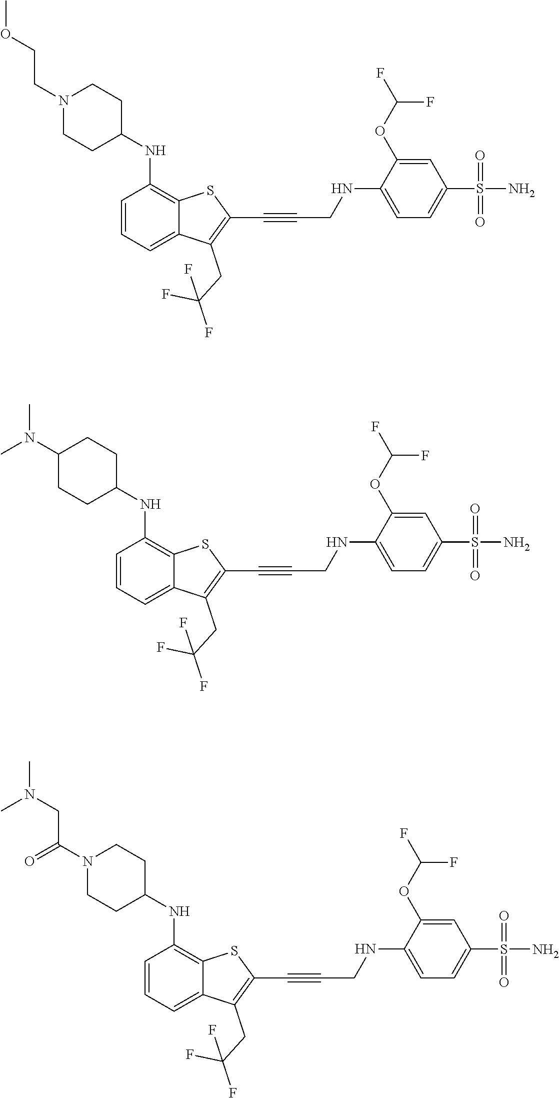 [SMILES: COCCN1CCC(Nc2cccc3c(CC(F)(F)F)c(C#CCNc4ccc(S(N)(=O)=O)cc4OC(F)F)sc23)CC1 ; CN(C)C1CCC(Nc2cccc3c(CC(F)(F)F)c(C#CCNc4ccc(S(N)(=O)=O)cc4OC(F)F)sc23)CC1 ; CN(C)CC(=O)N1CCC(Nc2cccc3c(CC(F)(F)F)c(C#CCNc4ccc(S(N)(=O)=O)cc4OC(F)F)sc23)CC1] 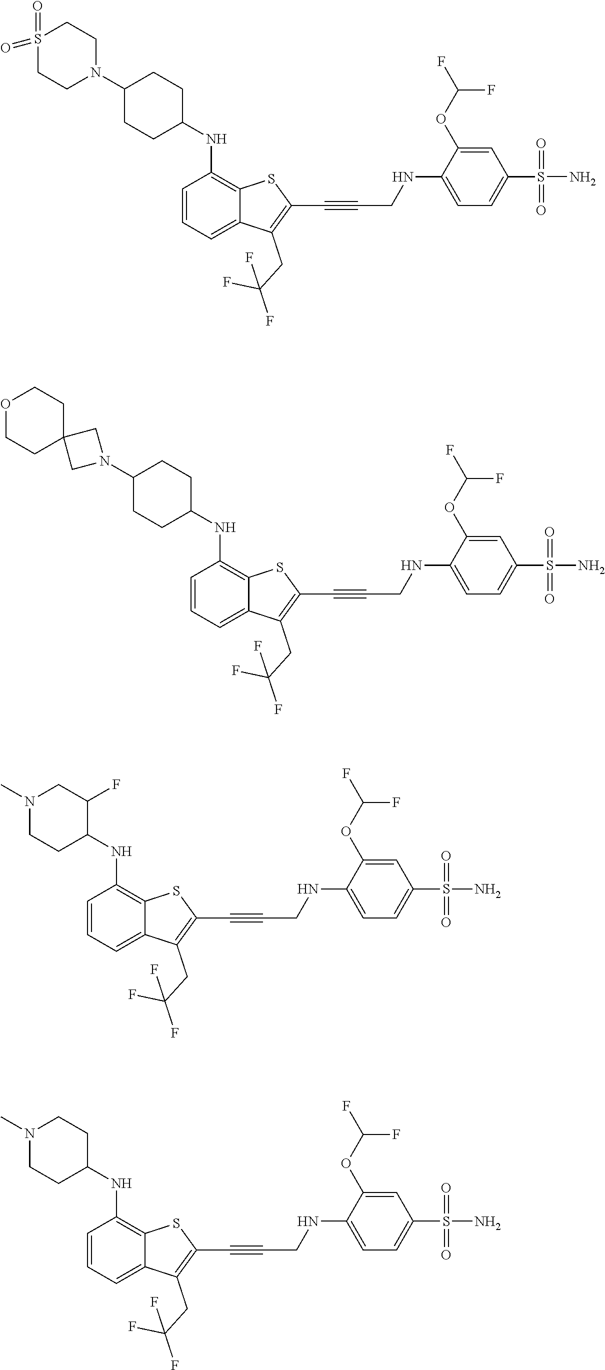 [SMILES: NS(=O)(=O)c1ccc(NCC#Cc2sc3c(NC4CCC(N5CCS(=O)(=O)CC5)CC4)cccc3c2CC(F)(F)F)c(OC(F)F)c1 ; NS(=O)(=O)c1ccc(NCC#Cc2sc3c(NC4CCC(N5CC6(CCOCC6)C5)CC4)cccc3c2CC(F)(F)F)c(OC(F)F)c1 ; CN1CCC(Nc2cccc3c(CC(F)(F)F)c(C#CCNc4ccc(S(N)(=O)=O)cc4OC(F)F)sc23)C(F)C1 ; CN1CCC(Nc2cccc3c(CC(F)(F)F)c(C#CCNc4ccc(S(N)(=O)=O)cc4OC(F)F)sc23)CC1] 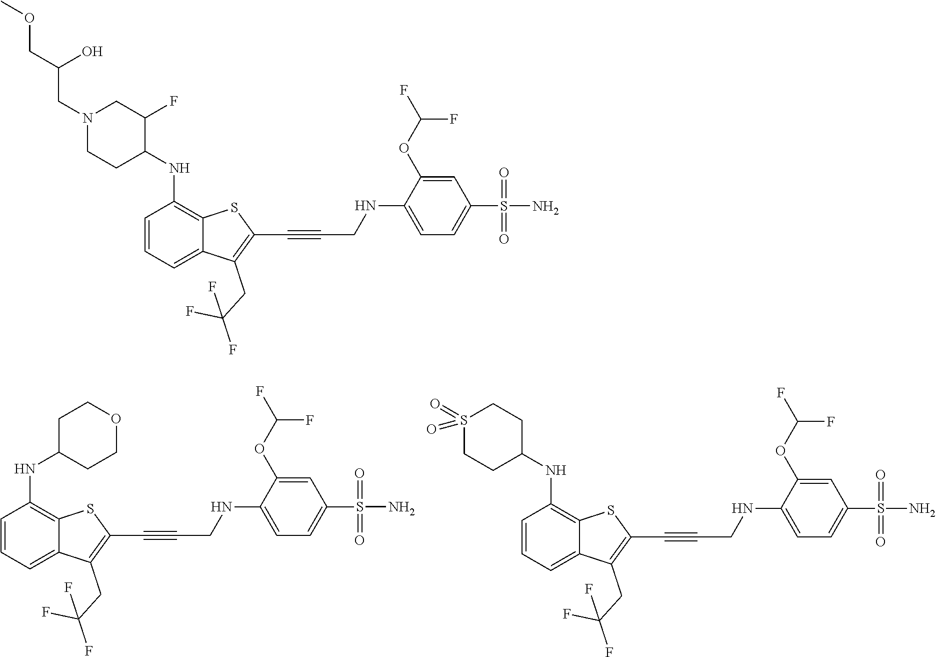 [SMILES: COCC(O)CN1CCC(Nc2cccc3c(CC(F)(F)F)c(C#CCNc4ccc(S(N)(=O)=O)cc4OC(F)F)sc23)C(F)C1 ; NS(=O)(=O)c1ccc(NCC#Cc2sc3c(NC4CCOCC4)cccc3c2CC(F)(F)F)c(OC(F)F)c1 ; NS(=O)(=O)c1ccc(NCC#Cc2sc3c(NC4CCS(=O)(=O)CC4)cccc3c2CC(F)(F)F)c(OC(F)F)c1] 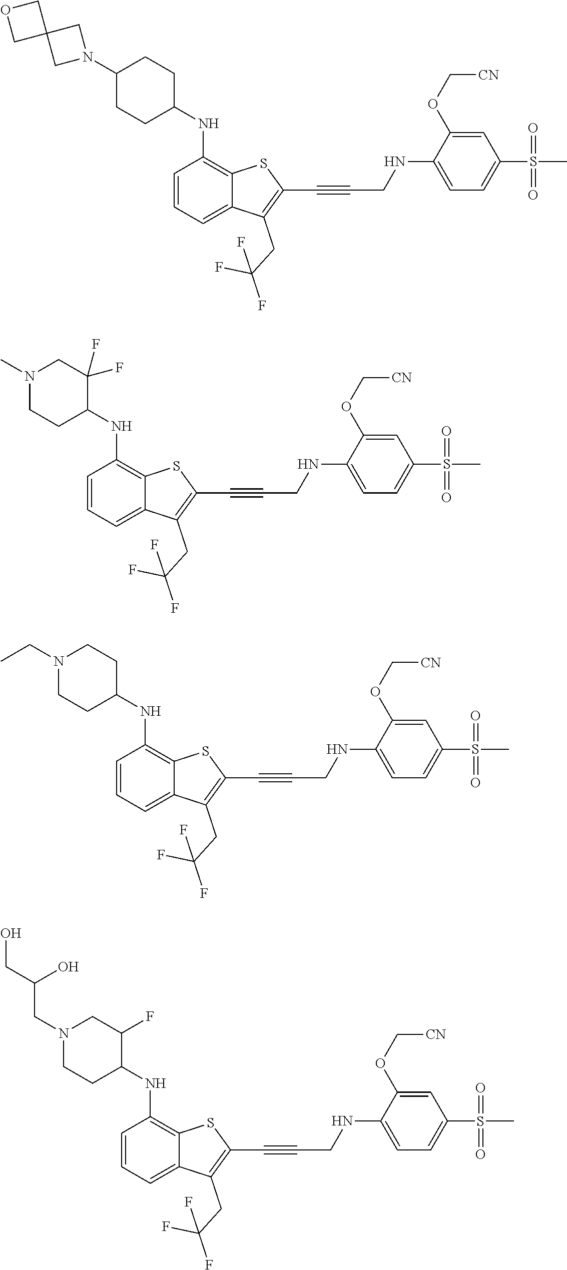 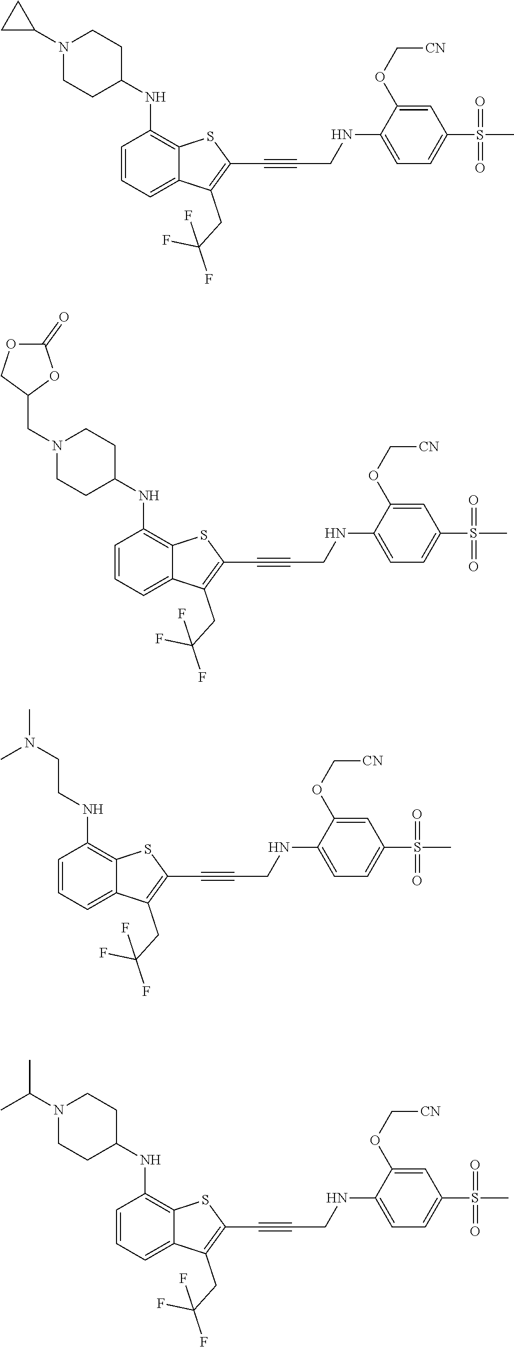 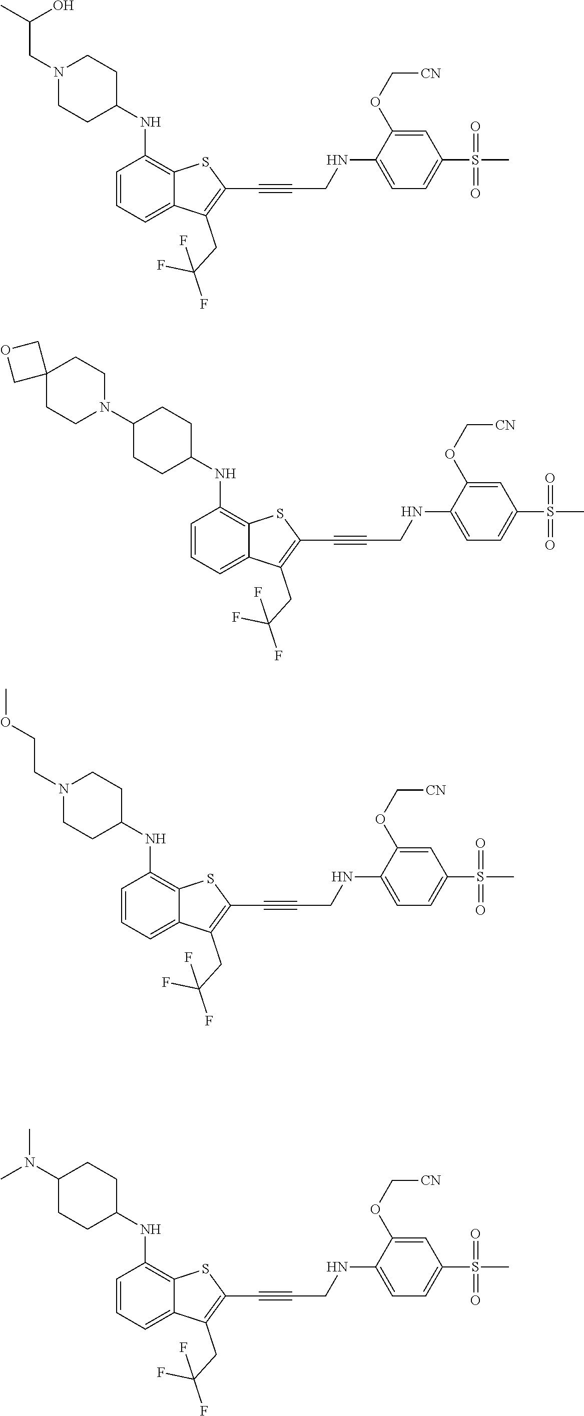 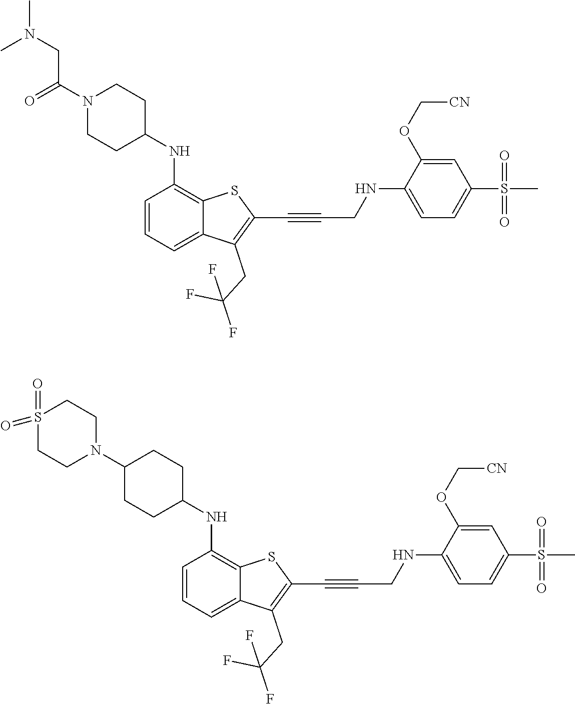 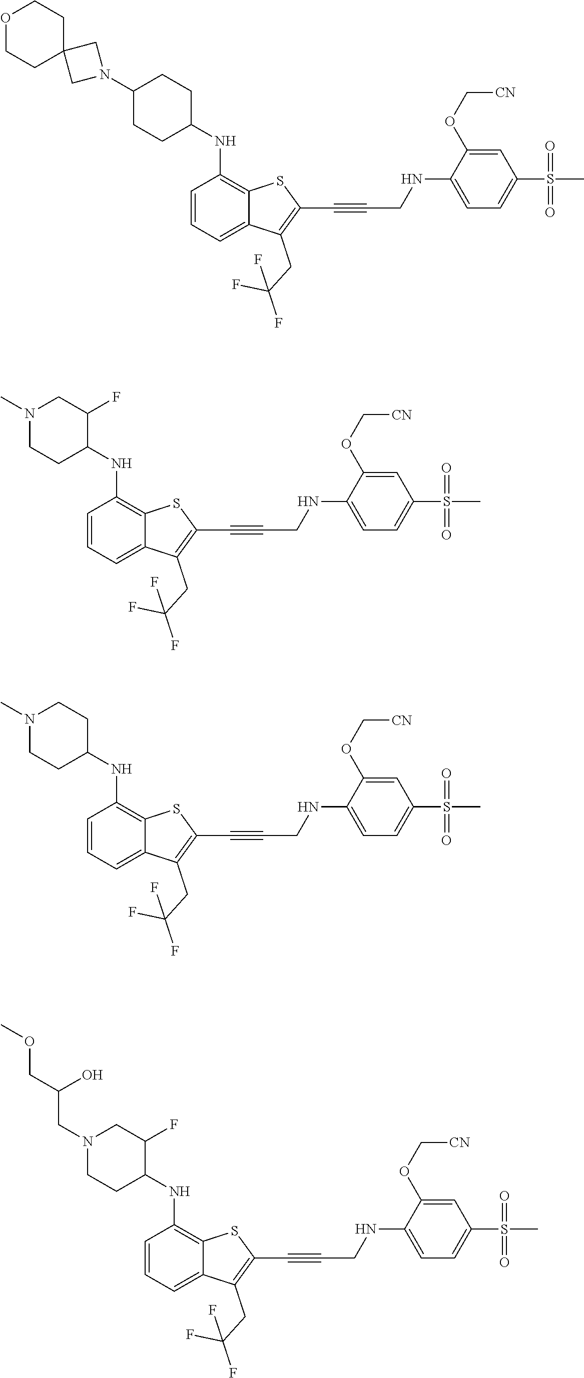 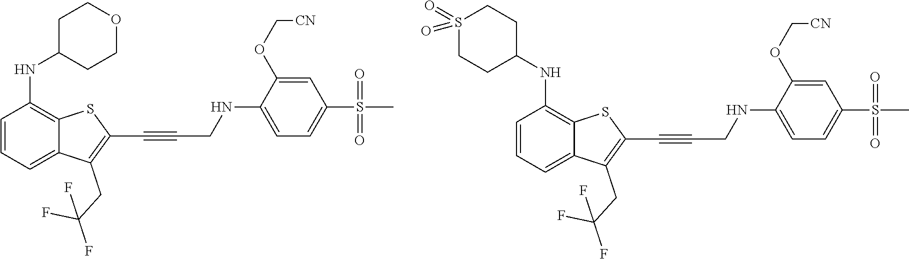 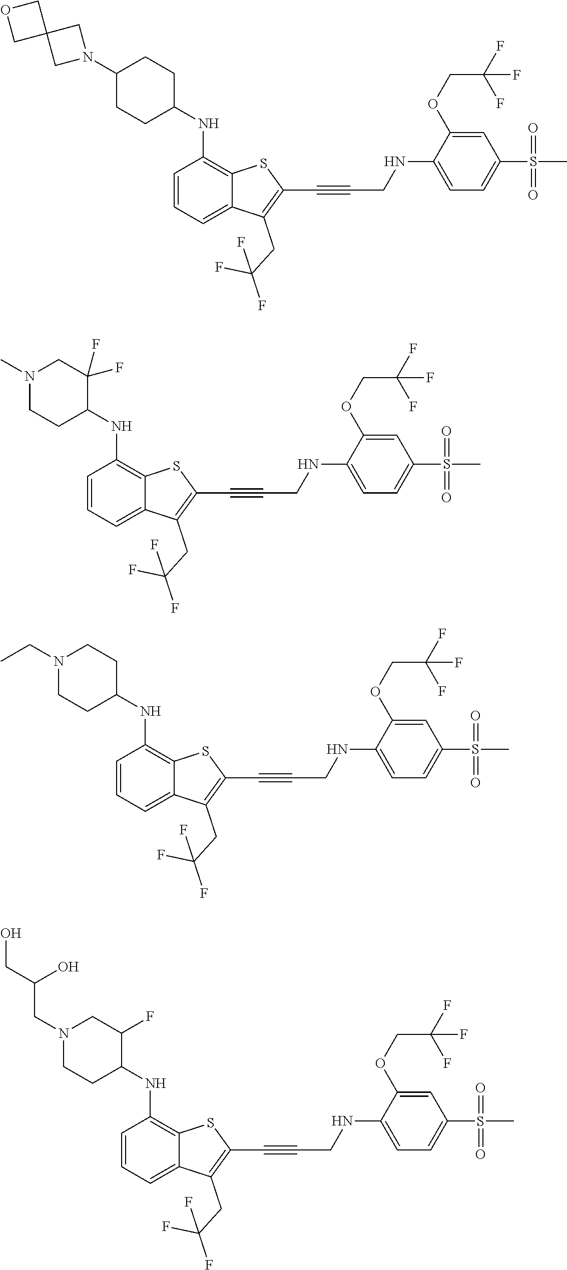 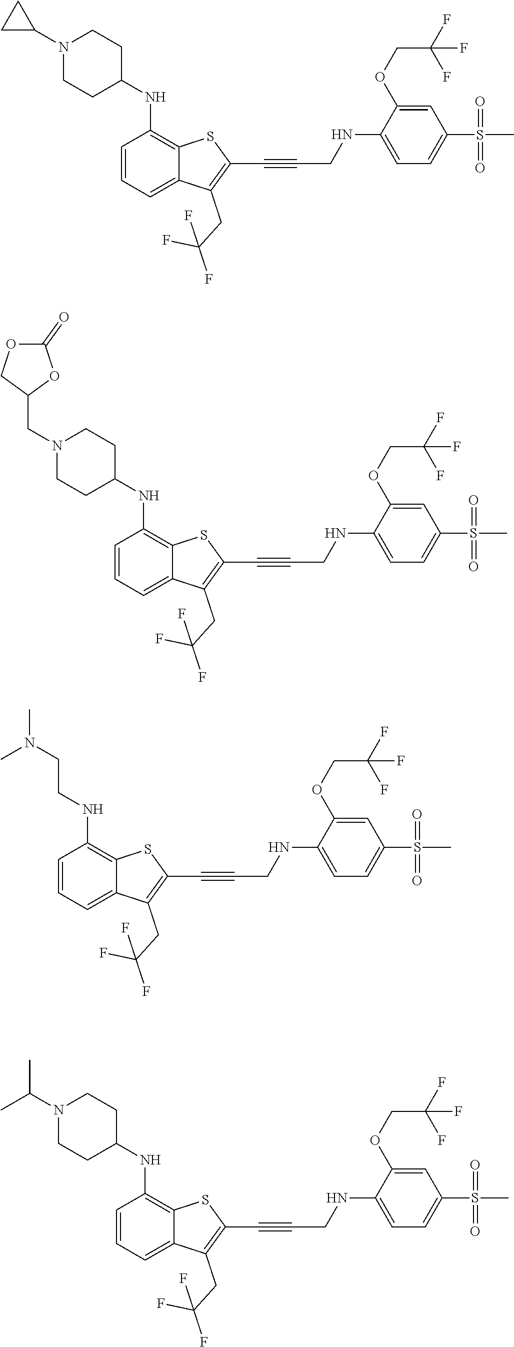 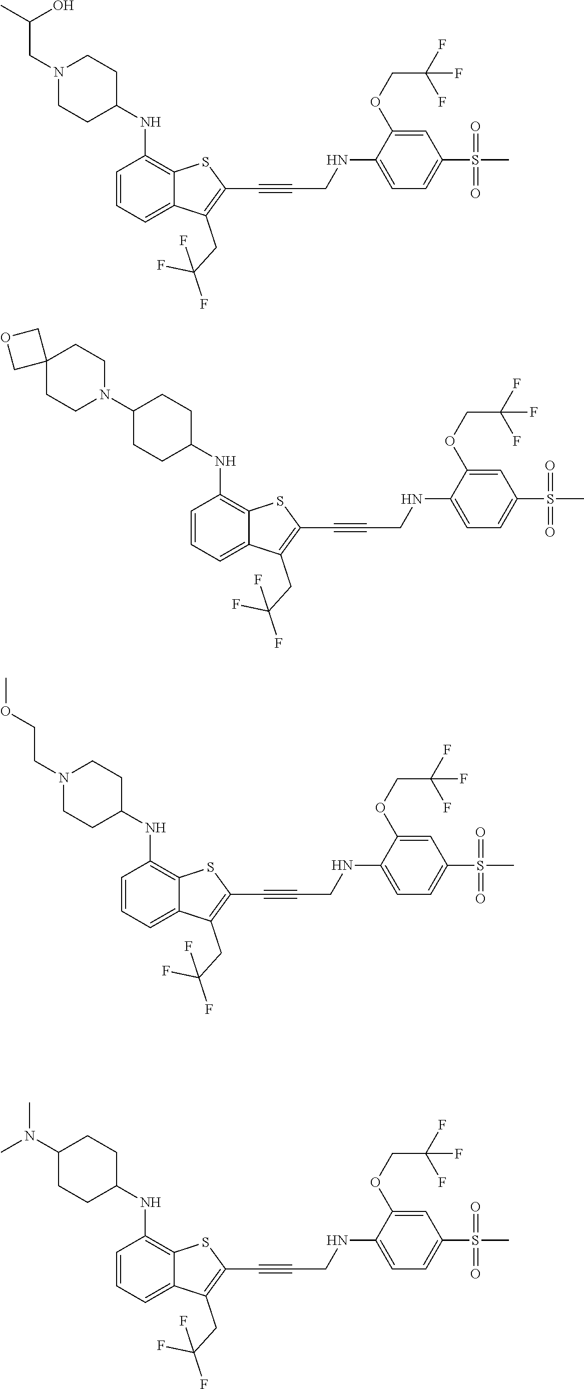 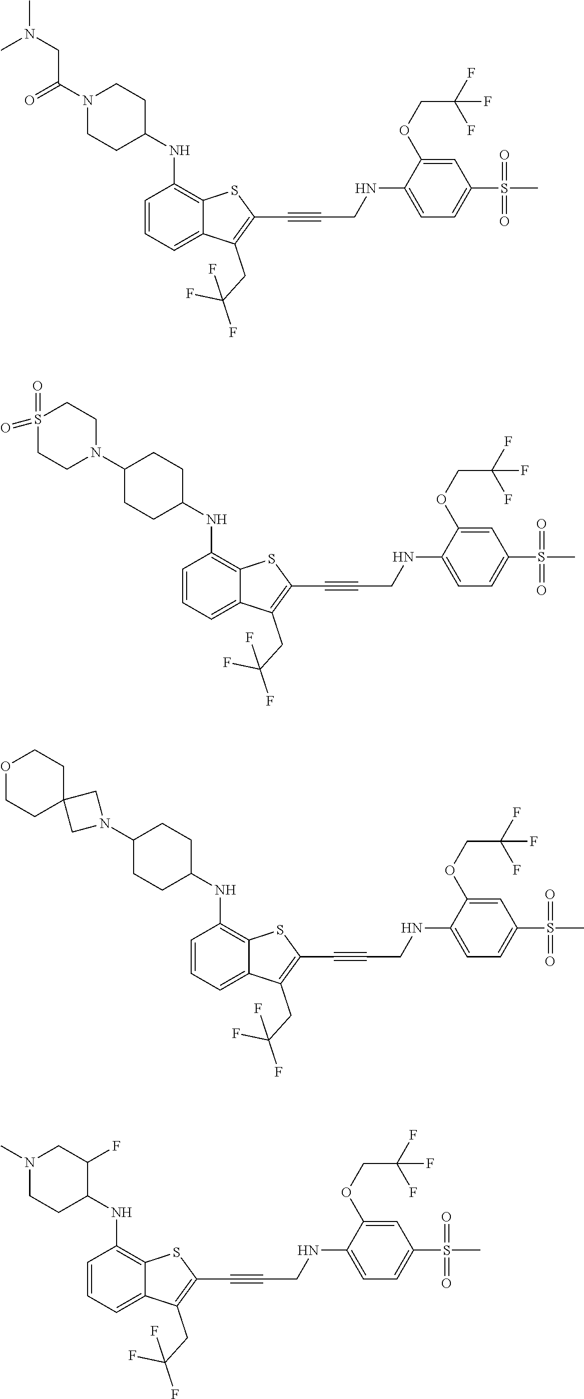 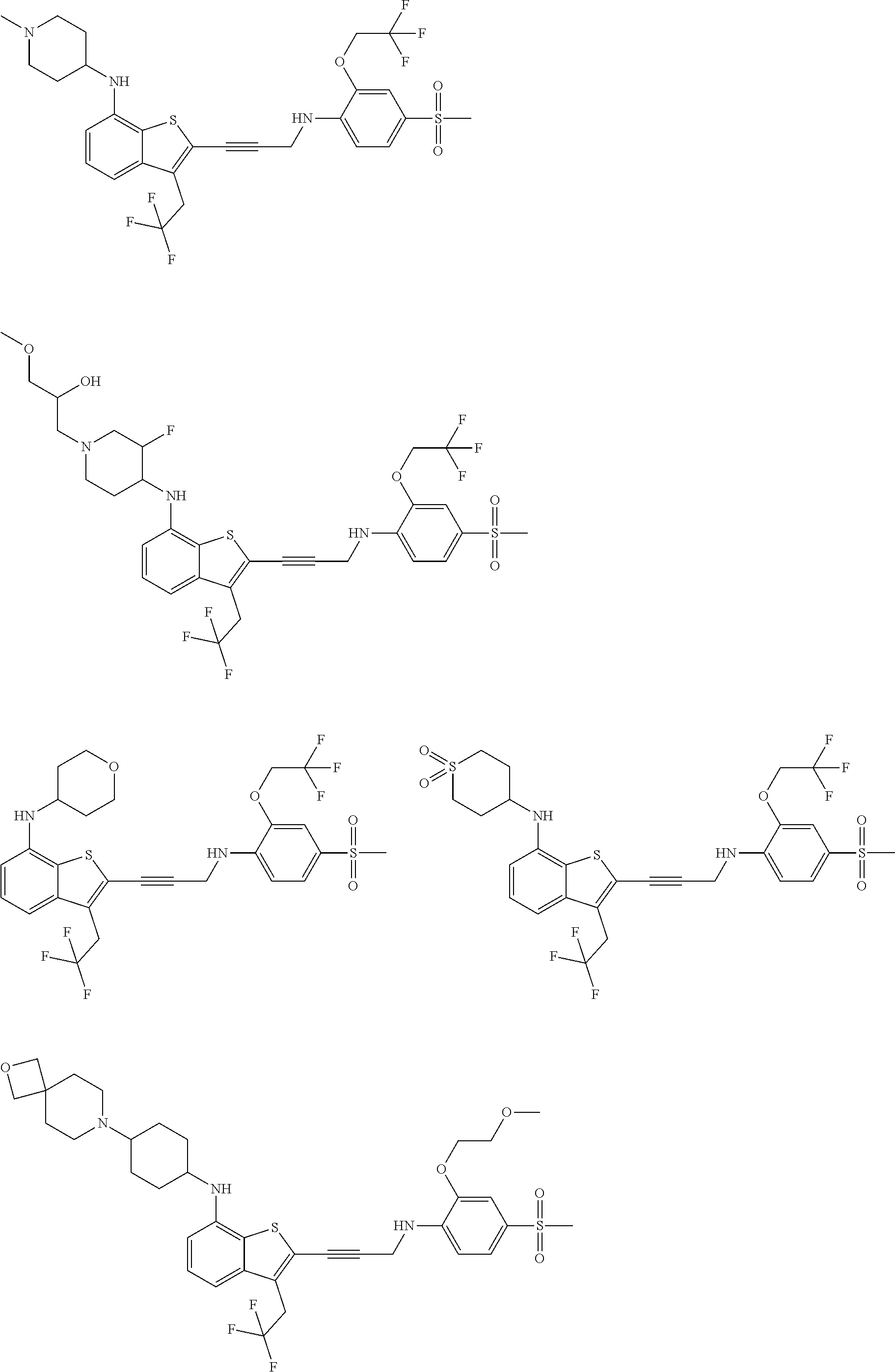 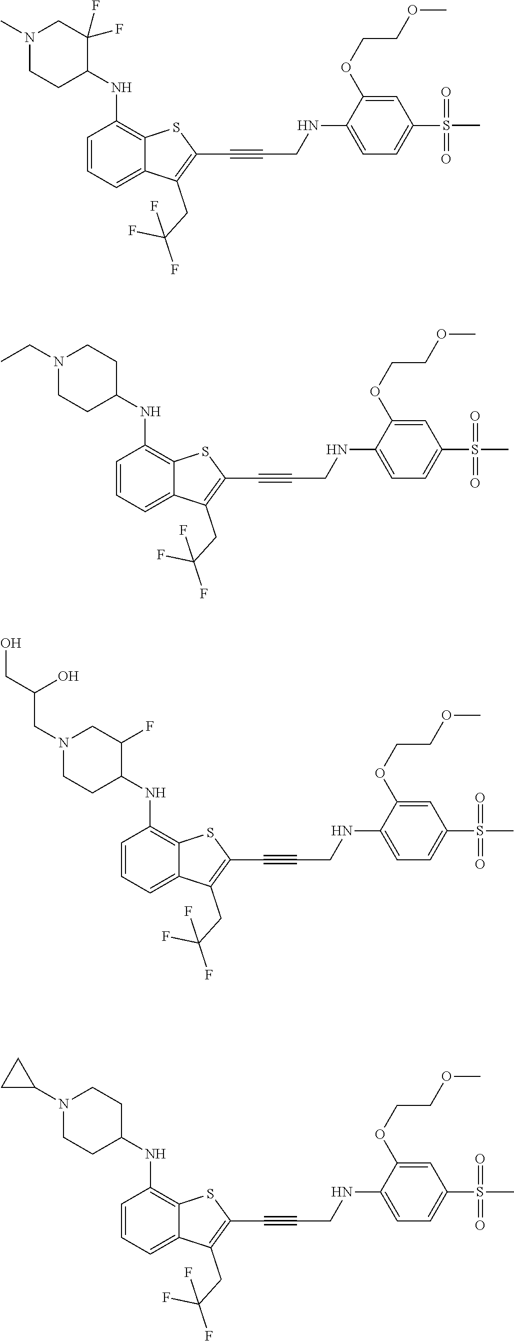 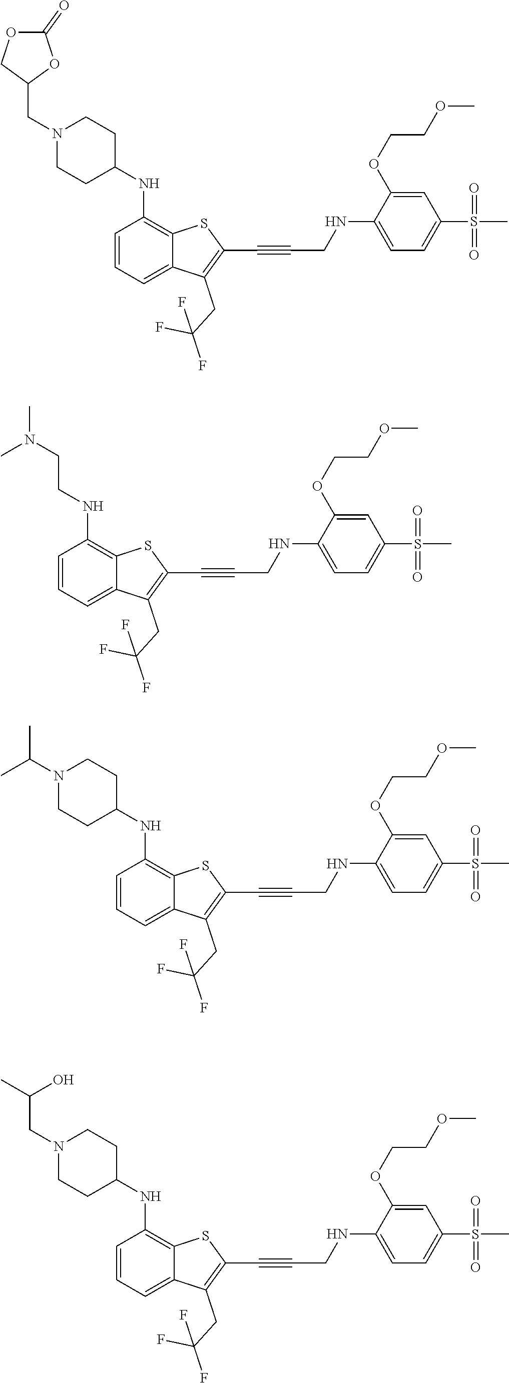 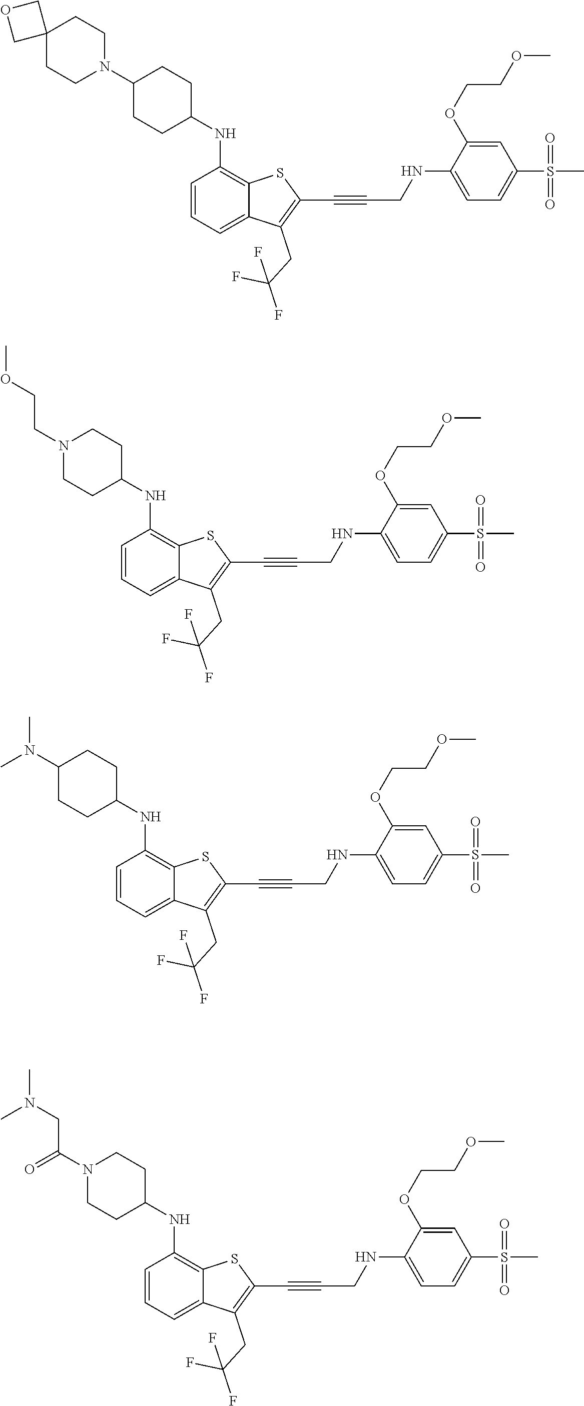 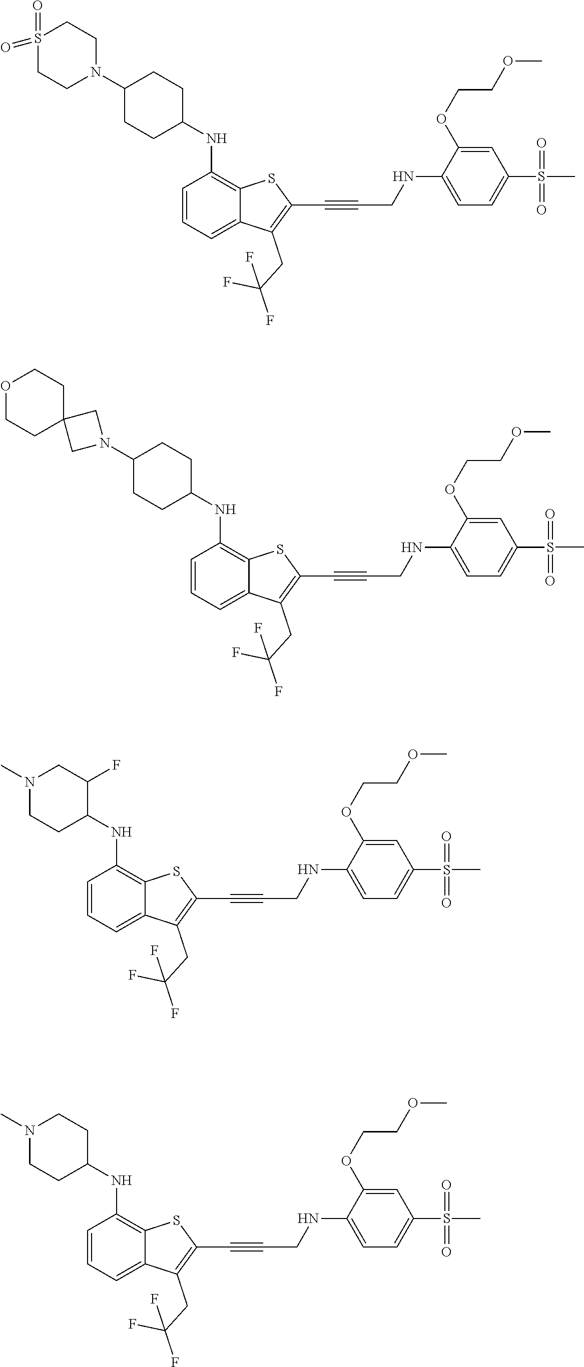 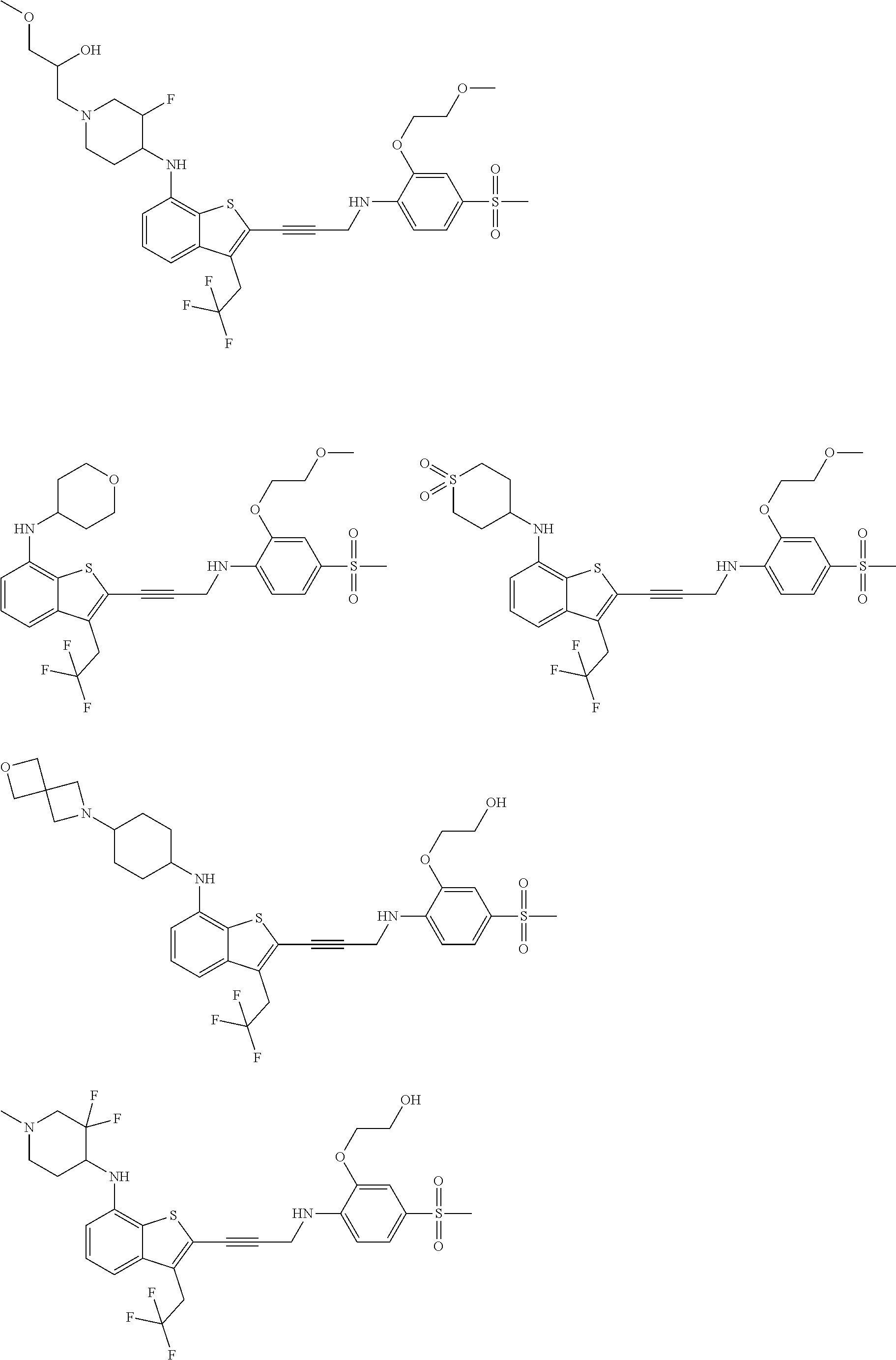 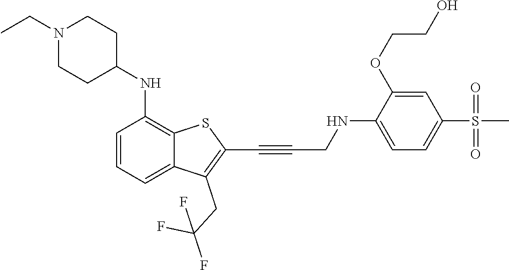 [SMILES: CCN1CCC(Nc2cccc3c(CC(F)(F)F)c(C#CCNc4ccc(S(C)(=O)=O)cc4OCCO)sc23)CC1] 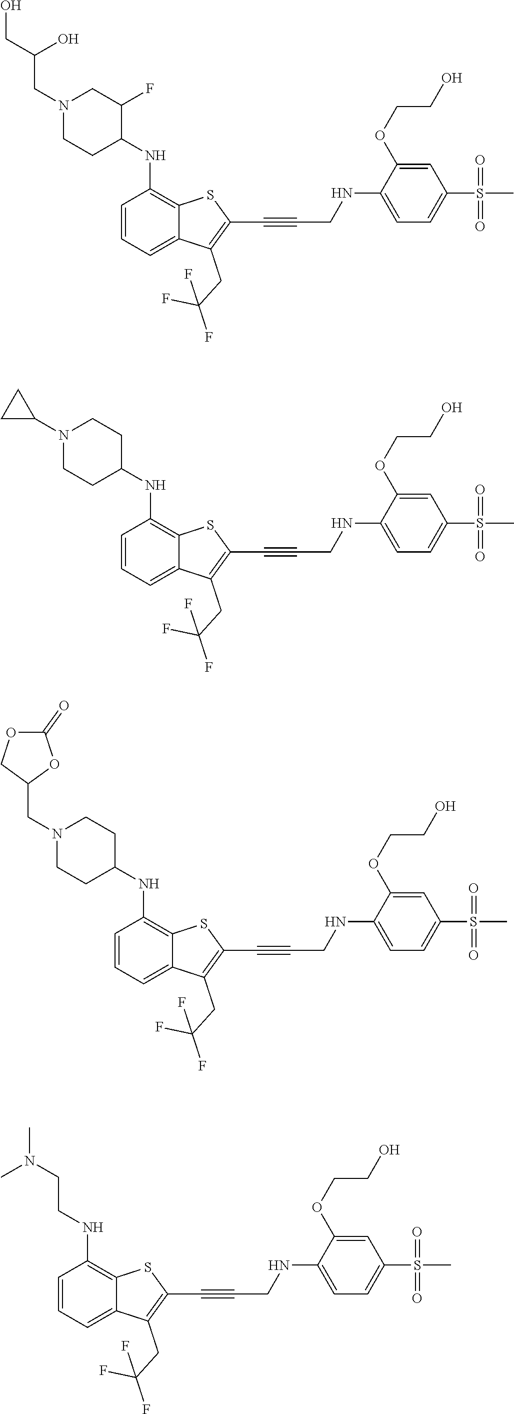 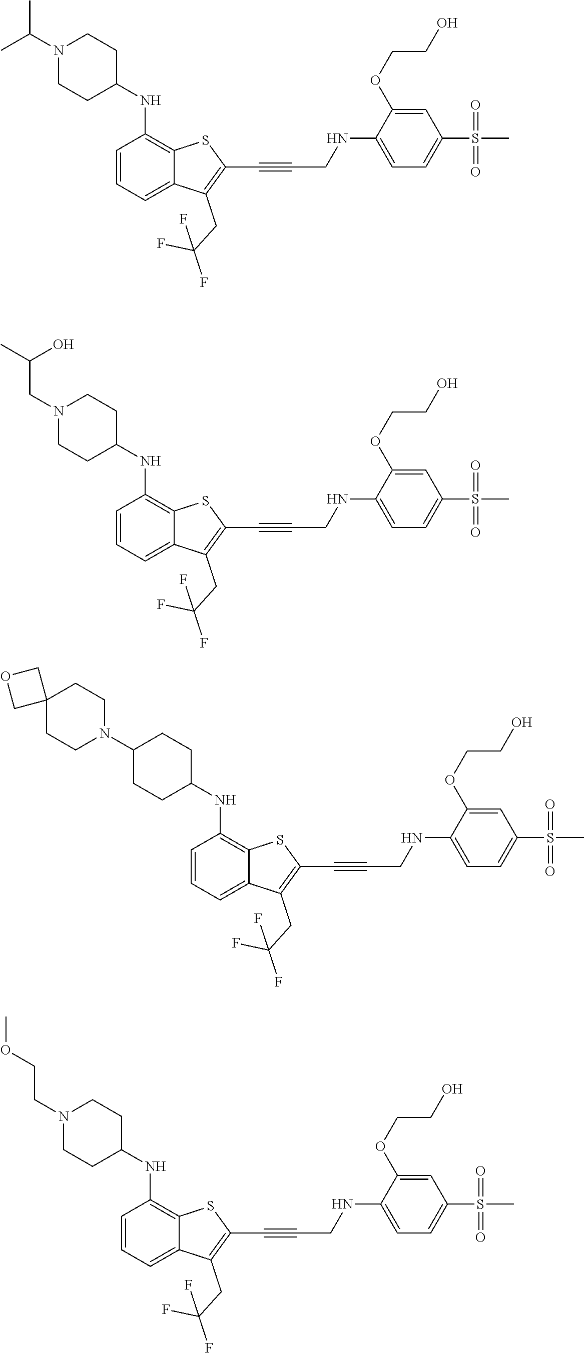 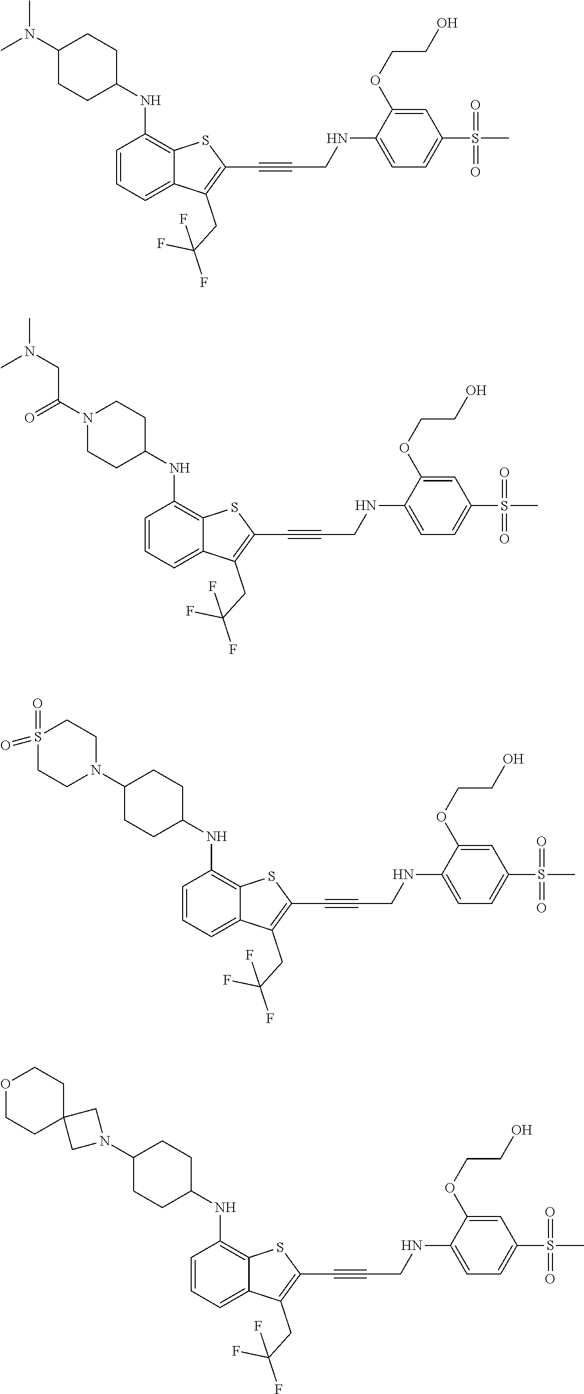 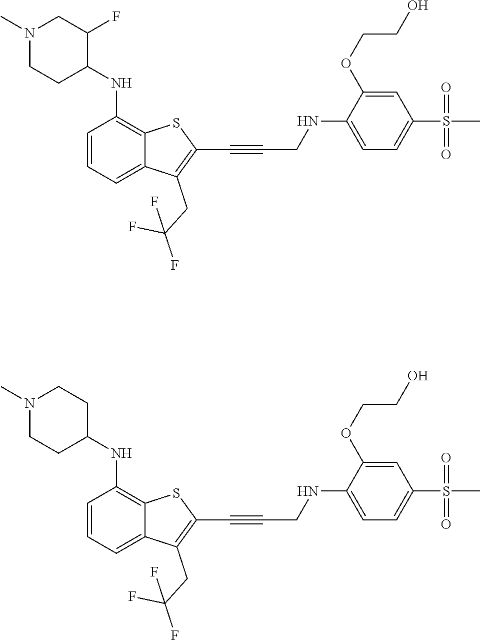 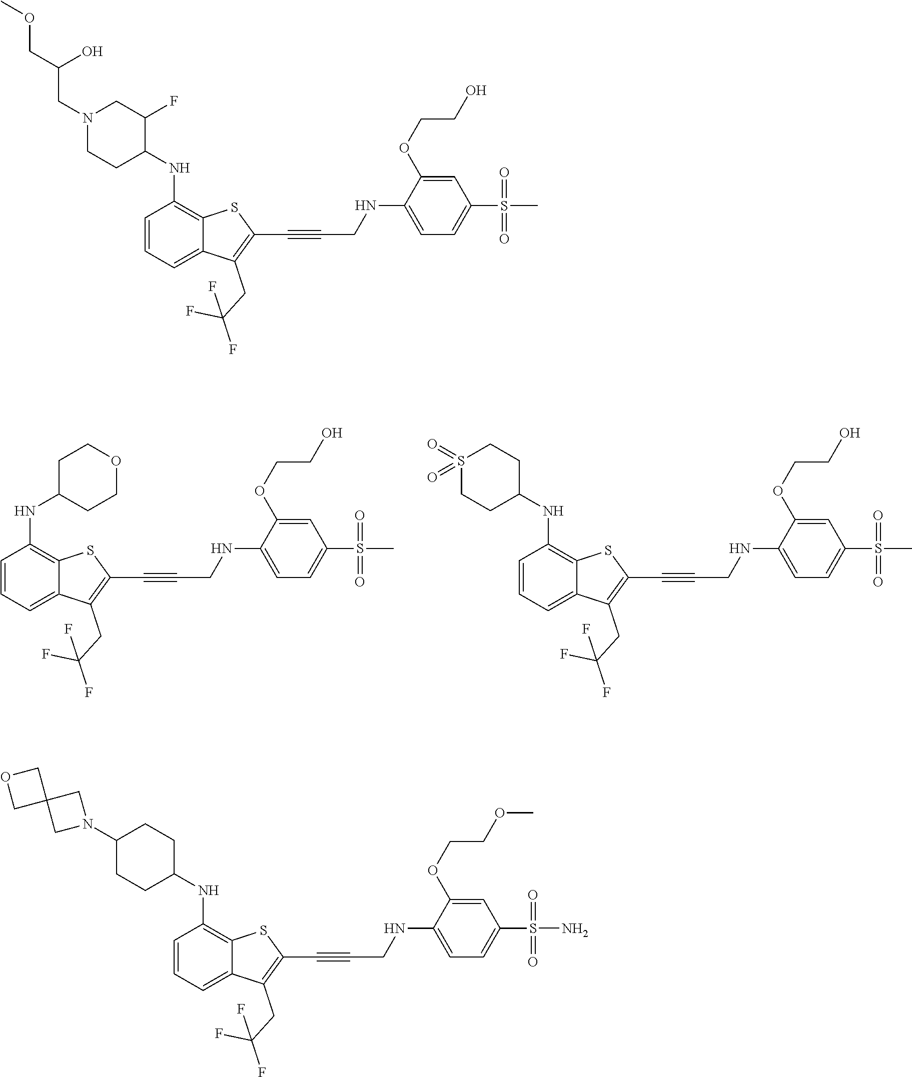 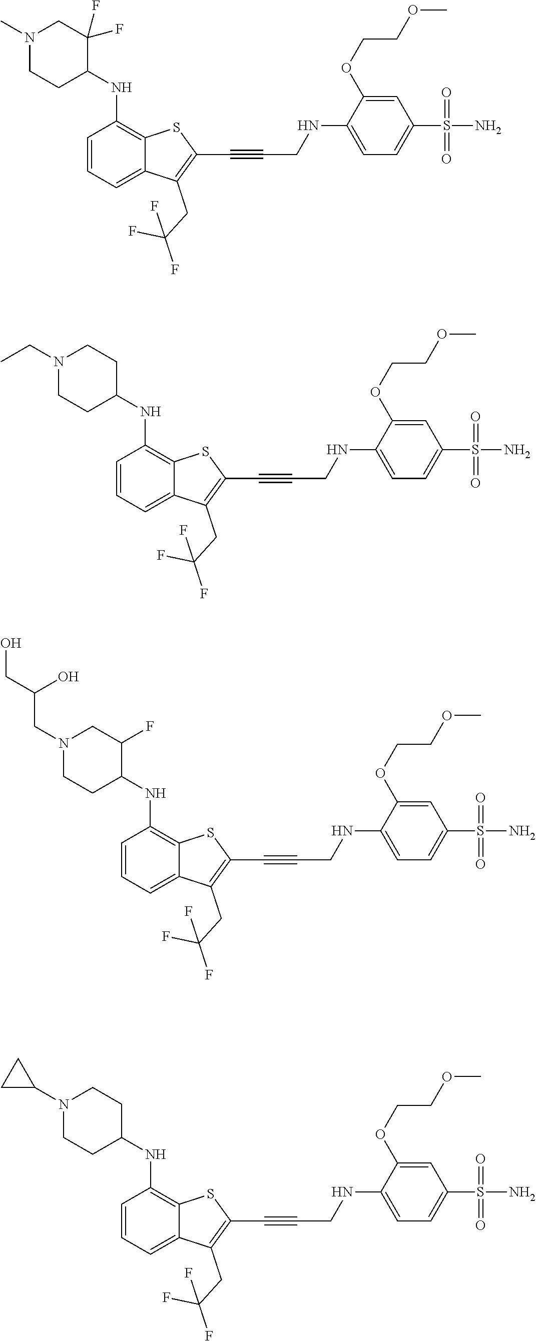 [SMILES: COCCOc1cc(S(N)(=O)=O)ccc1NCC#Cc1sc2c(NC3CCN(C)CC3(F)F)cccc2c1CC(F)(F)F ; CCN1CCC(Nc2cccc3c(CC(F)(F)F)c(C#CCNc4ccc(S(N)(=O)=O)cc4OCCOC)sc23)CC1 ; COCCOc1cc(S(N)(=O)=O)ccc1NCC#Cc1sc2c(NC3CCN(CC(O)CO)CC3F)cccc2c1CC(F)(F)F ; COCCOc1cc(S(N)(=O)=O)ccc1NCC#Cc1sc2c(NC3CCN(C4CC4)CC3)cccc2c1CC(F)(F)F] 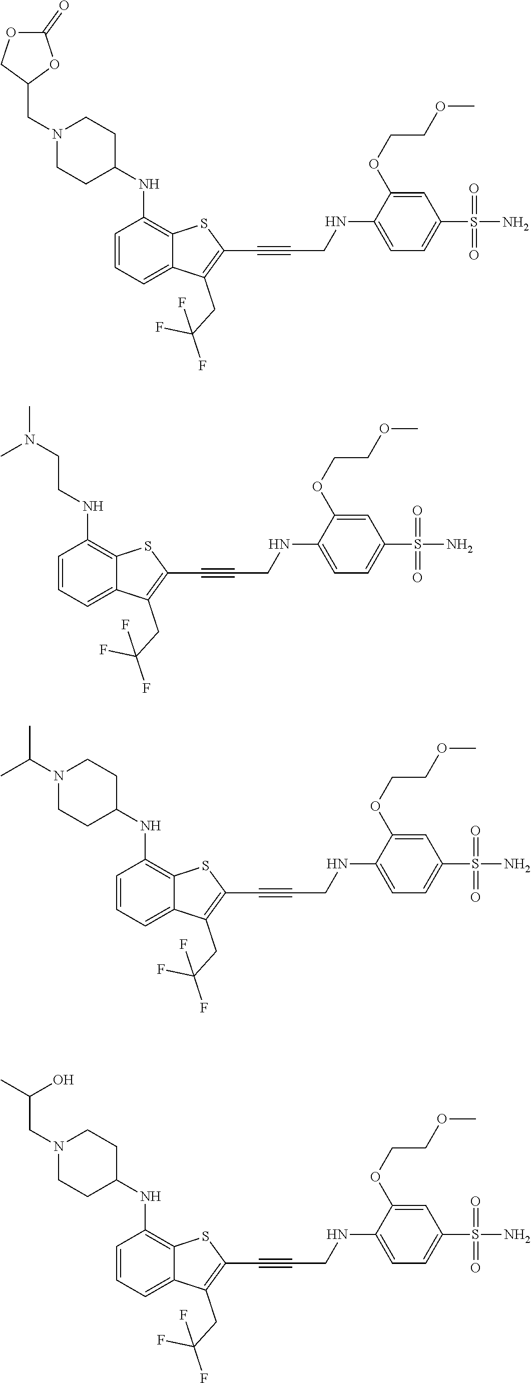 [SMILES: COCCOc1cc(S(N)(=O)=O)ccc1NCC#Cc1sc2c(NC3CCN(CC4COC(=O)O4)CC3)cccc2c1CC(F)(F)F ; COCCOc1cc(S(N)(=O)=O)ccc1NCC#Cc1sc2c(NCCN(C)C)cccc2c1CC(F)(F)F ; COCCOc1cc(S(N)(=O)=O)ccc1NCC#Cc1sc2c(NC3CCN(C(C)C)CC3)cccc2c1CC(F)(F)F ; COCCOc1cc(S(N)(=O)=O)ccc1NCC#Cc1sc2c(NC3CCN(CC(C)O)CC3)cccc2c1CC(F)(F)F] 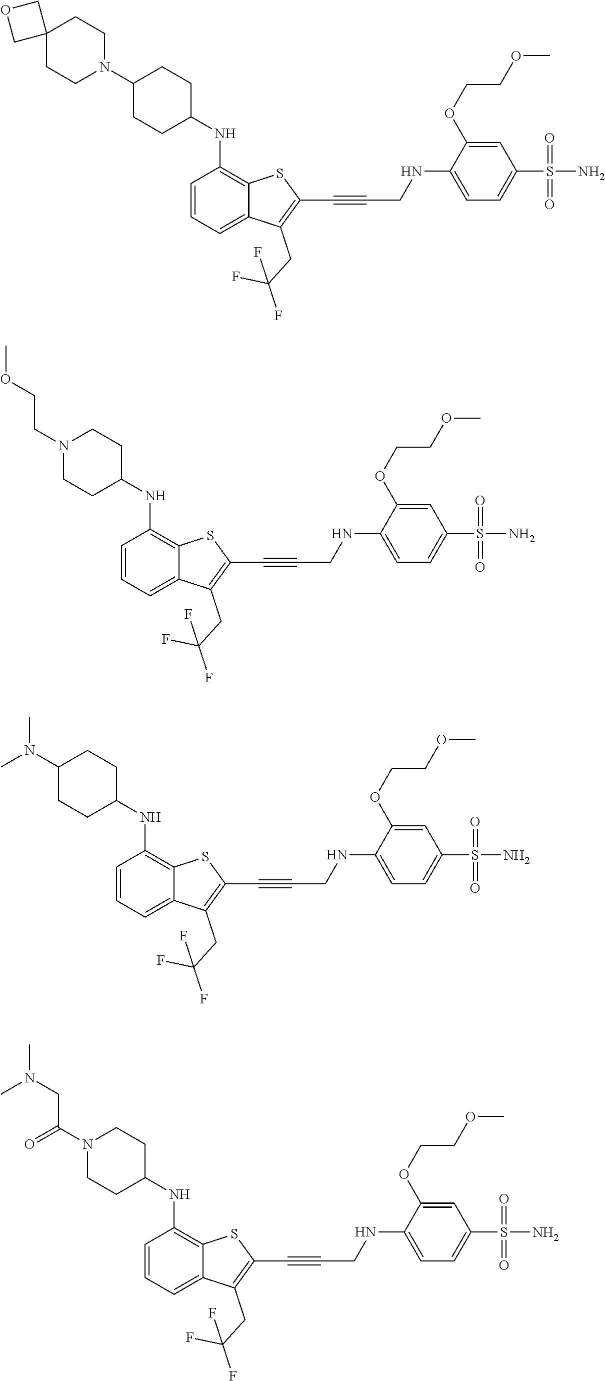 [SMILES: COCCOc1cc(S(N)(=O)=O)ccc1NCC#Cc1sc2c(NC3CCC(N4CCC5(CC4)COC5)CC3)cccc2c1CC(F)(F)F ; COCCOc1cc(S(N)(=O)=O)ccc1NCC#Cc1sc2c(NC3CCN(CCOC)CC3)cccc2c1CC(F)(F)F ; COCCOc1cc(S(N)(=O)=O)ccc1NCC#Cc1sc2c(NC3CCC(N(C)C)CC3)cccc2c1CC(F)(F)F ; COCCOc1cc(S(N)(=O)=O)ccc1NCC#Cc1sc2c(NC3CCN(C(=O)CN(C)C)CC3)cccc2c1CC(F)(F)F] 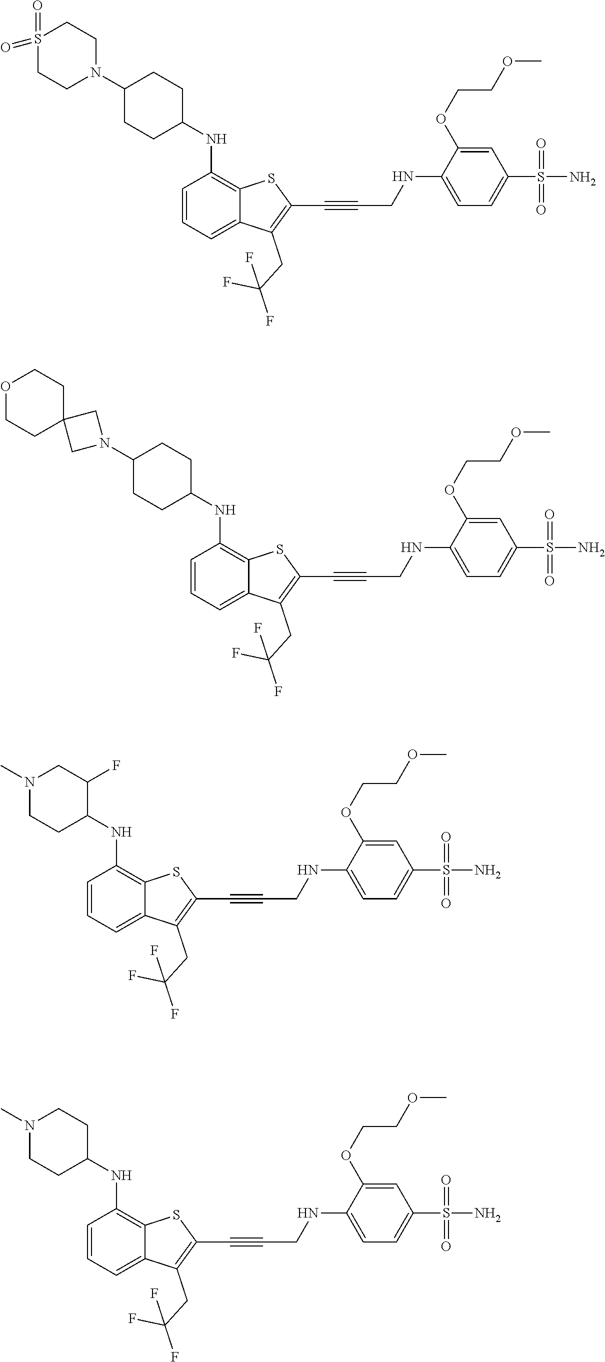 [SMILES: COCCOc1cc(S(N)(=O)=O)ccc1NCC#Cc1sc2c(NC3CCC(N4CCS(=O)(=O)CC4)CC3)cccc2c1CC(F)(F)F ; COCCOc1cc(S(N)(=O)=O)ccc1NCC#Cc1sc2c(NC3CCC(N4CC5(CCOCC5)C4)CC3)cccc2c1CC(F)(F)F ; COCCOc1cc(S(N)(=O)=O)ccc1NCC#Cc1sc2c(NC3CCN(C)CC3F)cccc2c1CC(F)(F)F ; COCCOc1cc(S(N)(=O)=O)ccc1NCC#Cc1sc2c(NC3CCN(C)CC3)cccc2c1CC(F)(F)F] 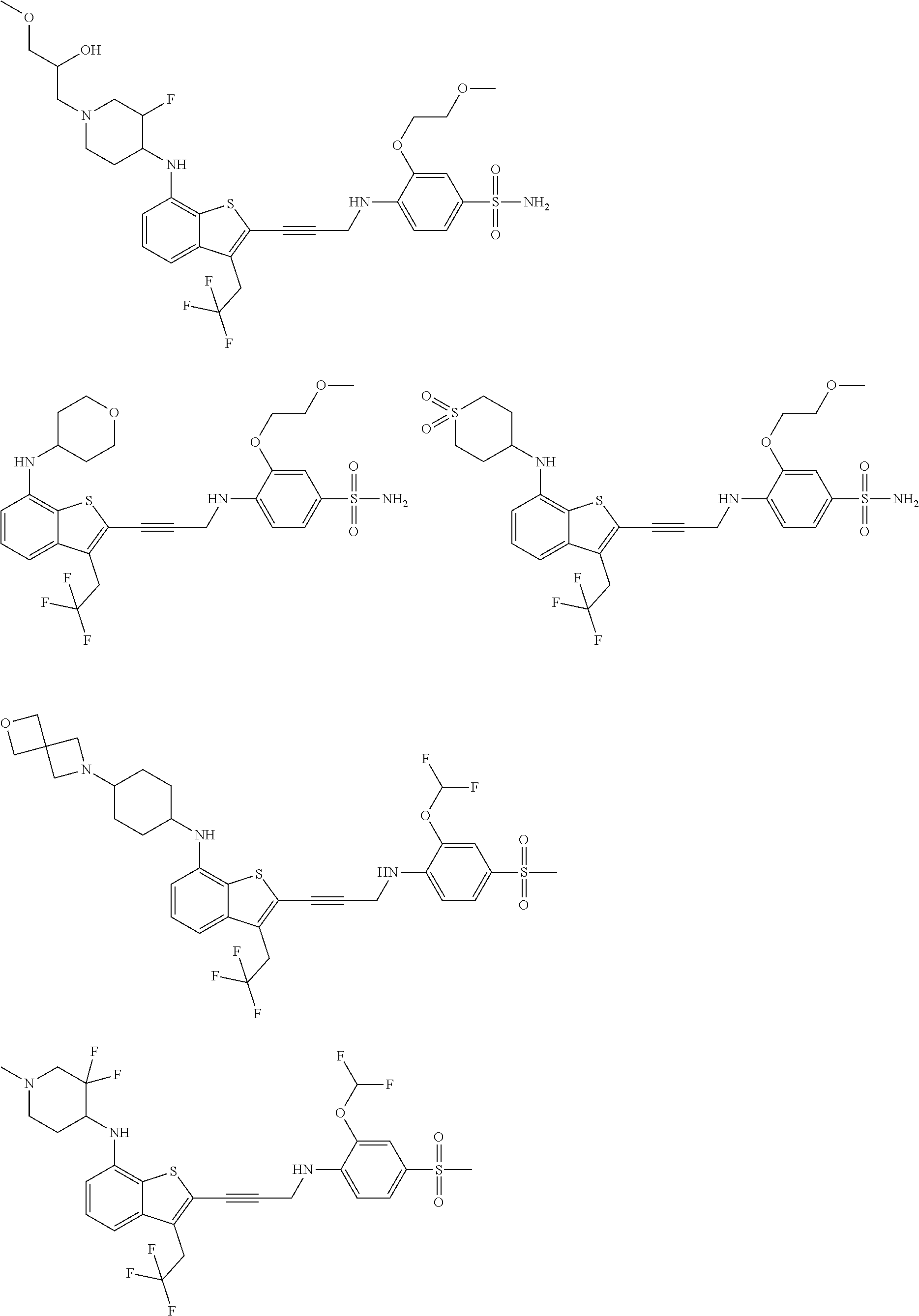 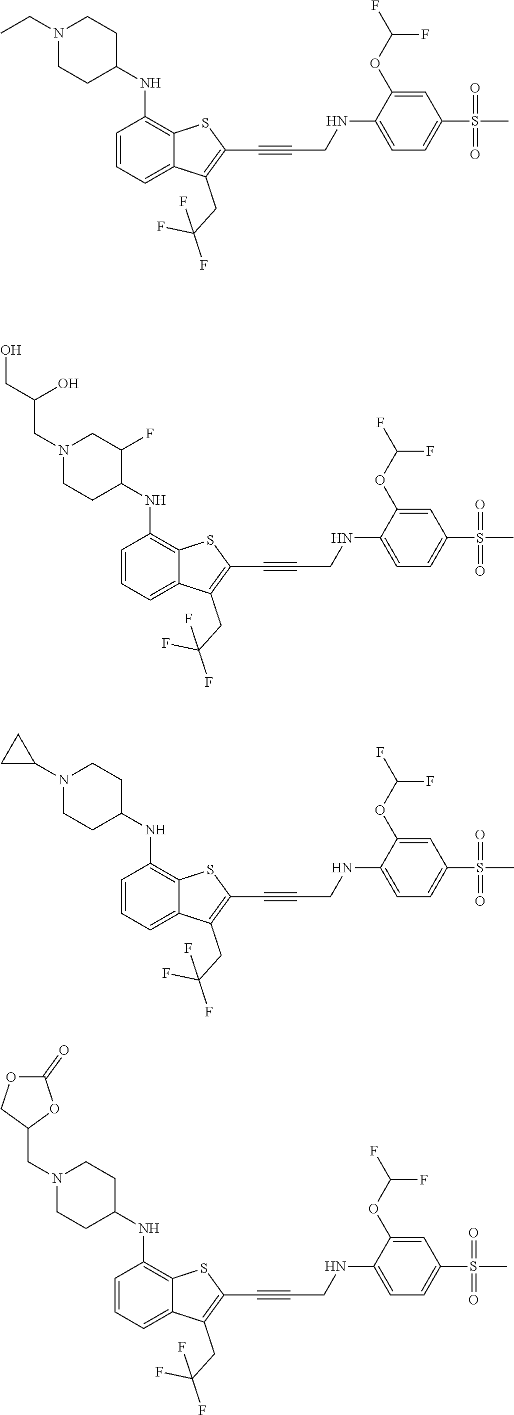 [SMILES: CCN1CCC(Nc2cccc3c(CC(F)(F)F)c(C#CCNc4ccc(S(C)(=O)=O)cc4OC(F)F)sc23)CC1 ; CS(=O)(=O)c1ccc(NCC#Cc2sc3c(NC4CCN(CC(O)CO)CC4F)cccc3c2CC(F)(F)F)c(OC(F)F)c1 ; CS(=O)(=O)c1ccc(NCC#Cc2sc3c(NC4CCN(C5CC5)CC4)cccc3c2CC(F)(F)F)c(OC(F)F)c1 ; CS(=O)(=O)c1ccc(NCC#Cc2sc3c(NC4CCN(CC5COC(=O)O5)CC4)cccc3c2CC(F)(F)F)c(OC(F)F)c1] 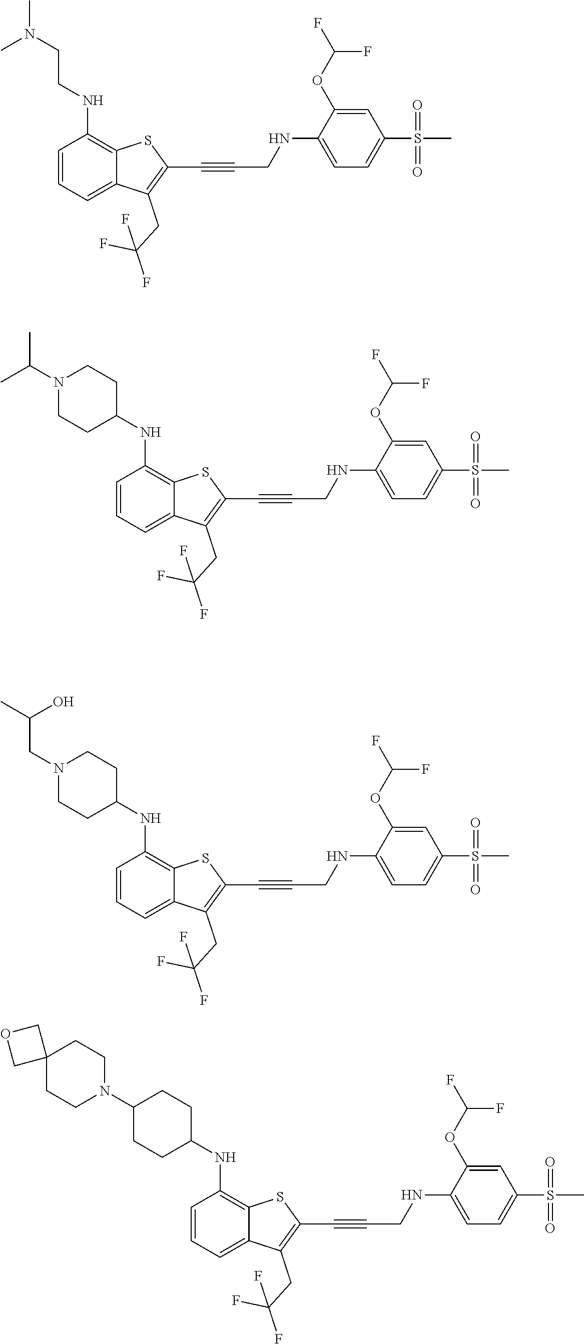 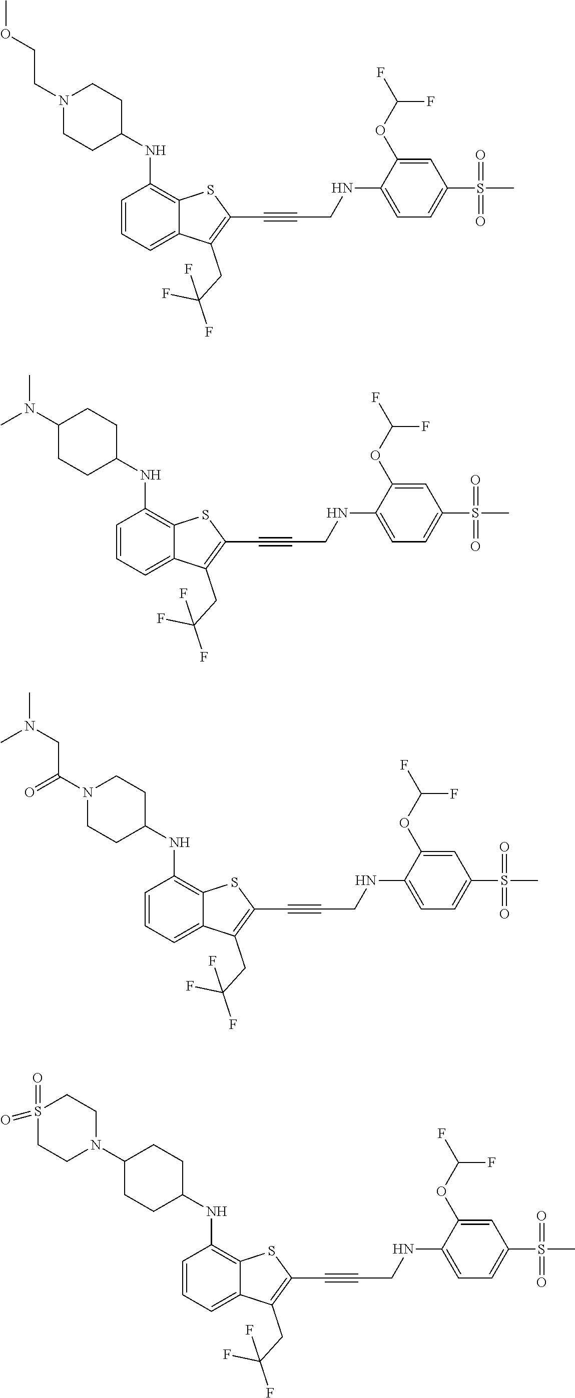 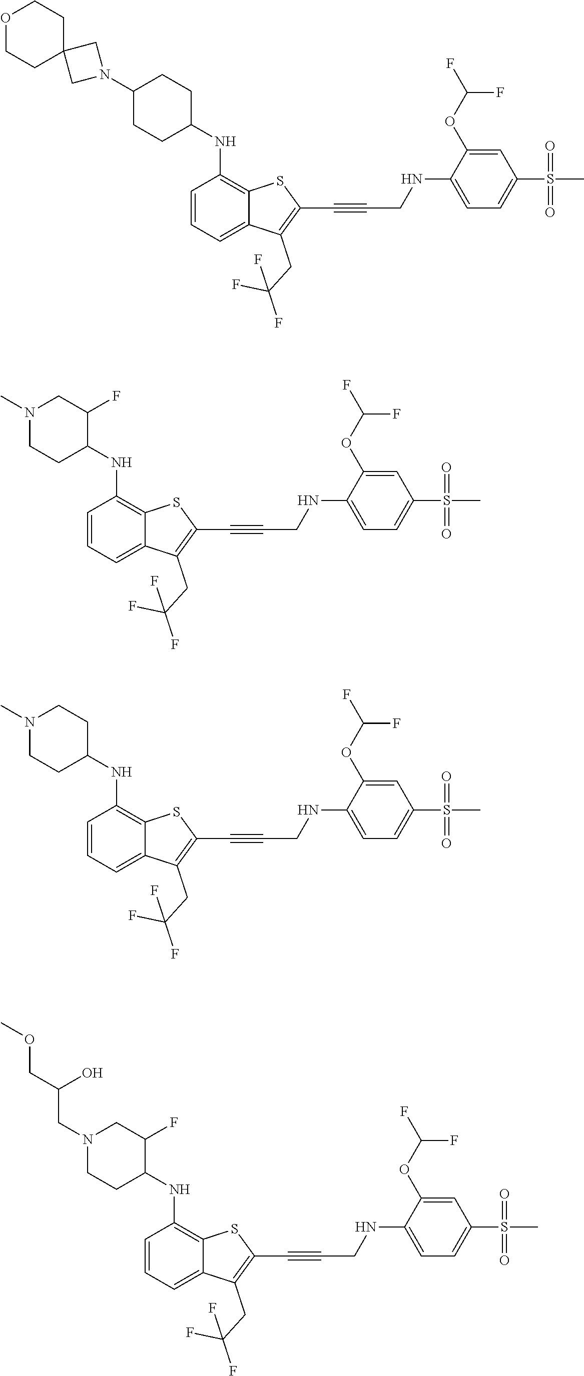 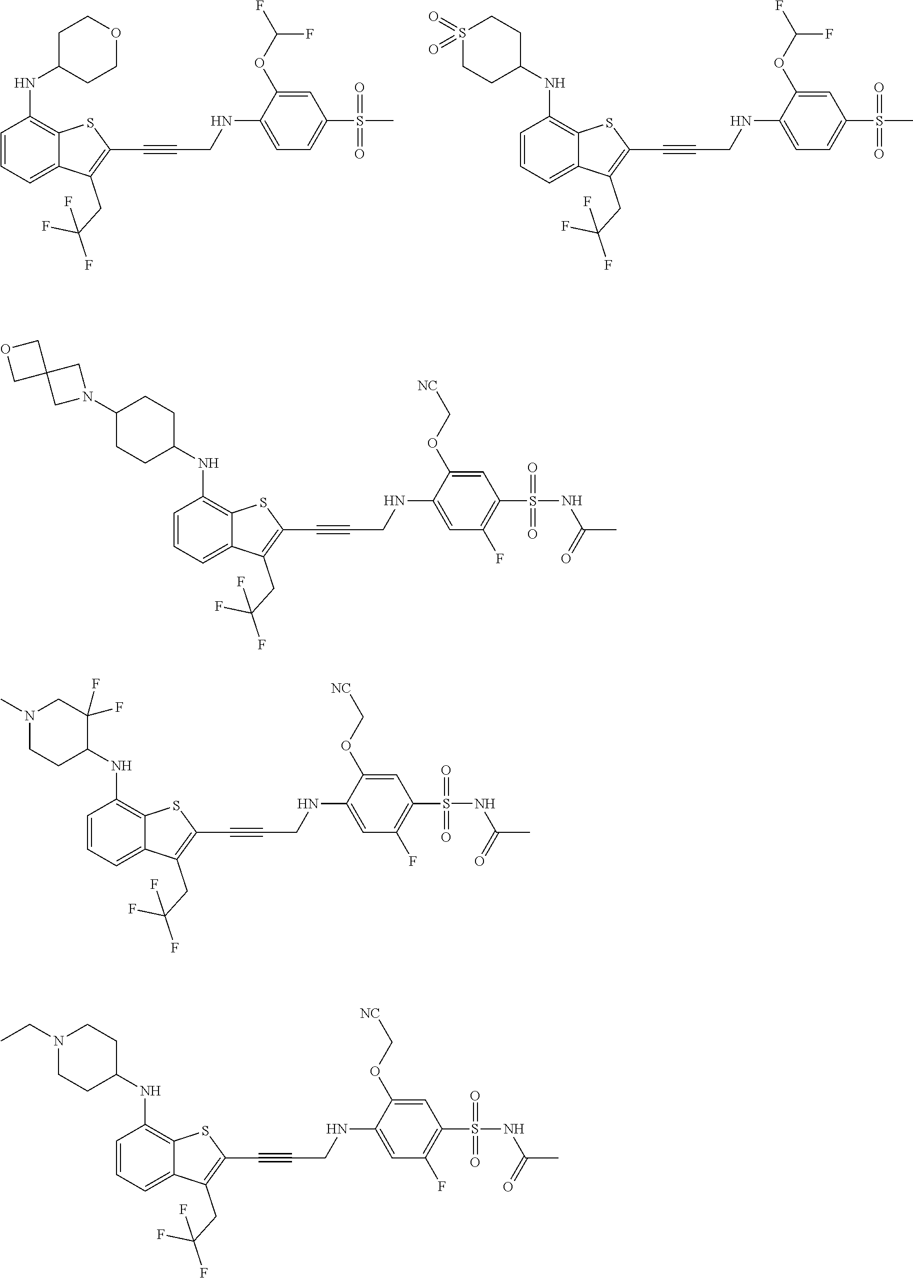 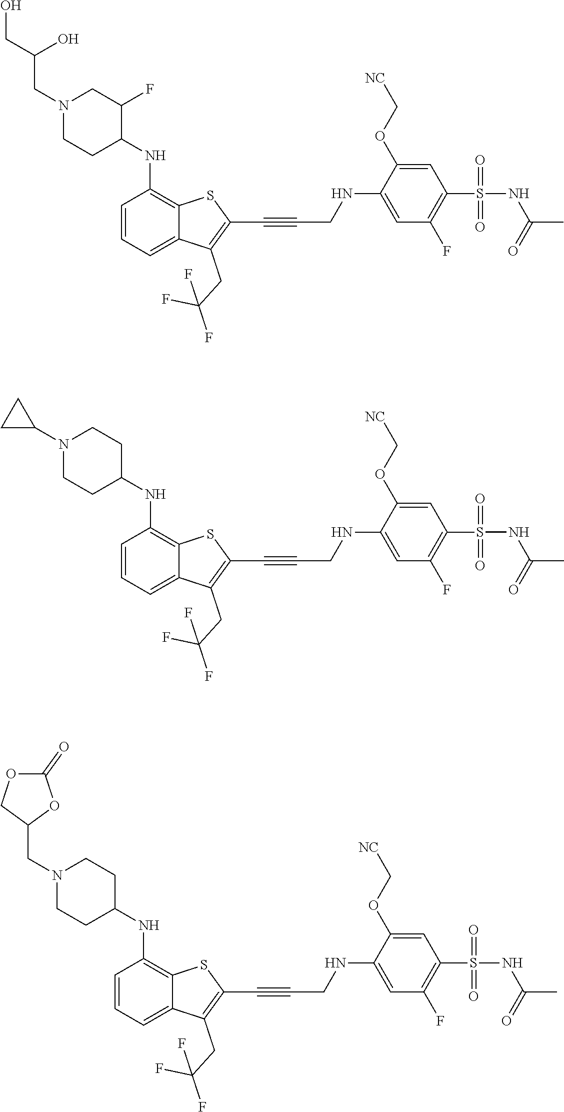 [SMILES: CC(=O)NS(=O)(=O)c1cc(OCC#N)c(NCC#Cc2sc3c(NC4CCN(CC(O)CO)CC4F)cccc3c2CC(F)(F)F)cc1F ; CC(=O)NS(=O)(=O)c1cc(OCC#N)c(NCC#Cc2sc3c(NC4CCN(C5CC5)CC4)cccc3c2CC(F)(F)F)cc1F ; CC(=O)NS(=O)(=O)c1cc(OCC#N)c(NCC#Cc2sc3c(NC4CCN(CC5COC(=O)O5)CC4)cccc3c2CC(F)(F)F)cc1F] 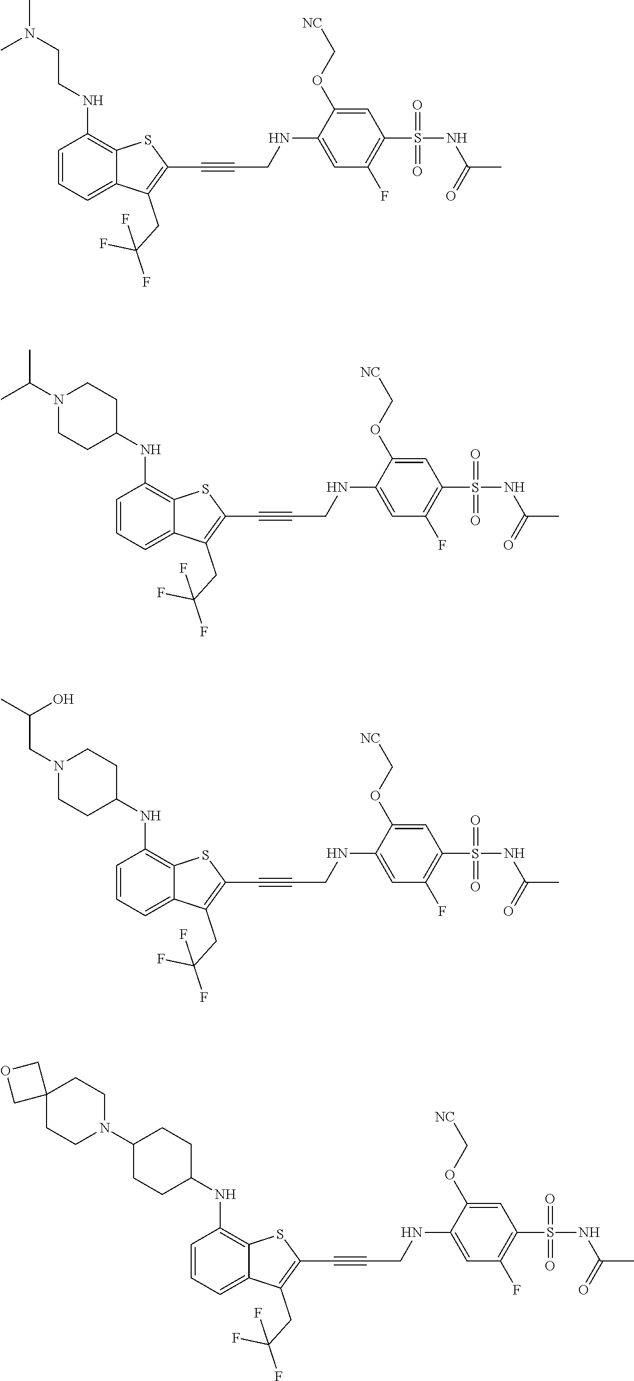 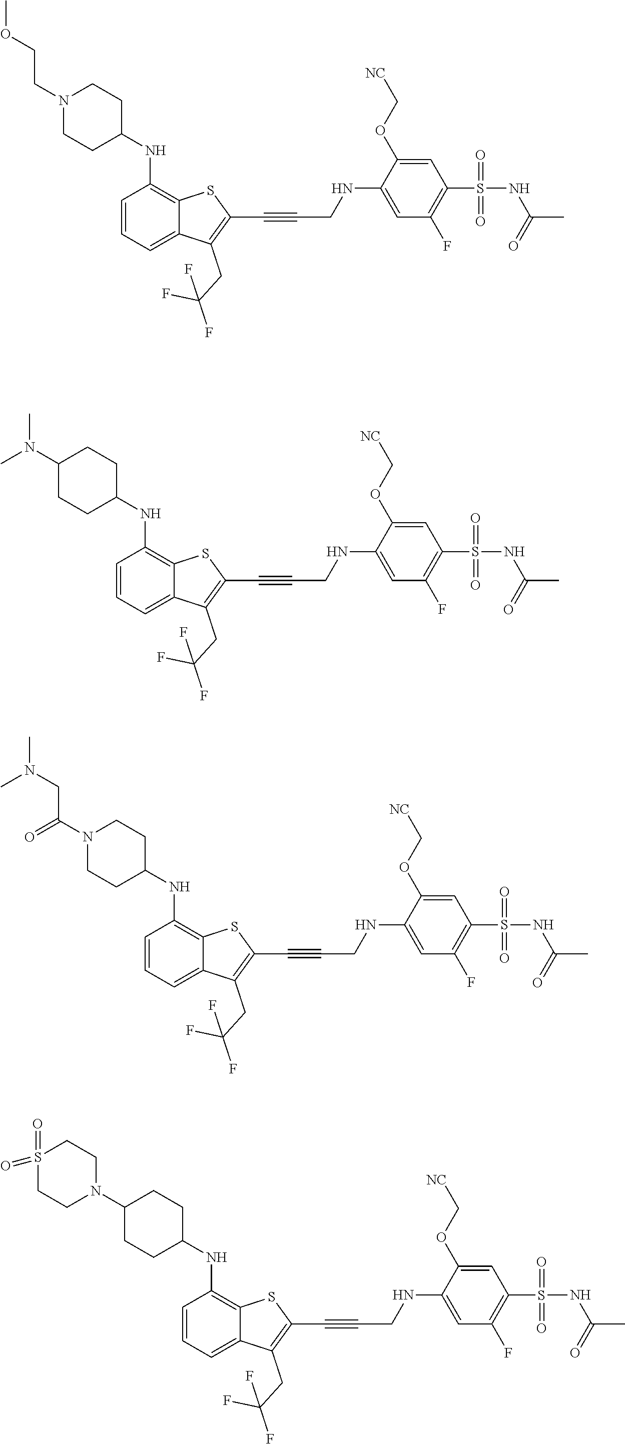 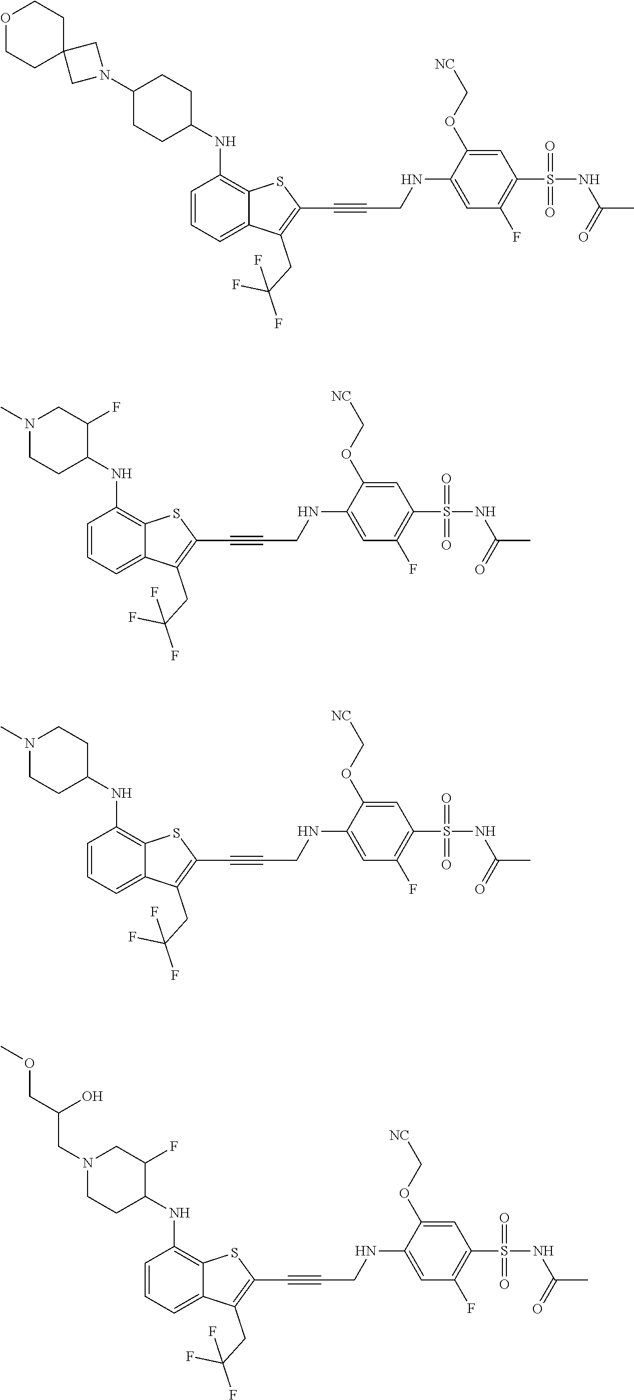 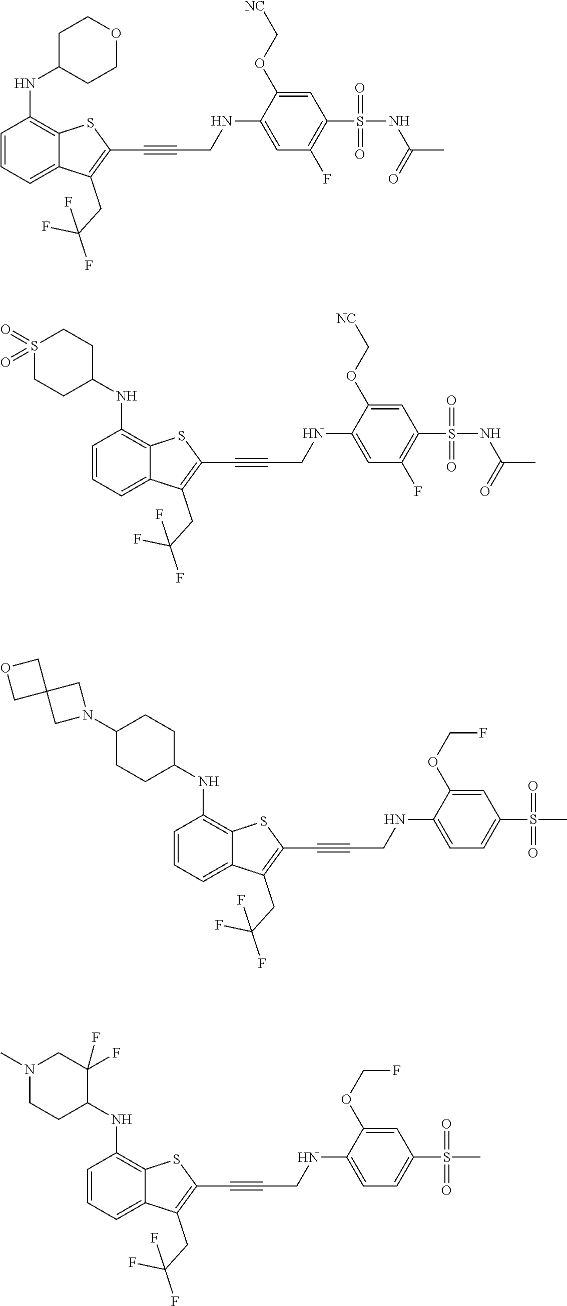 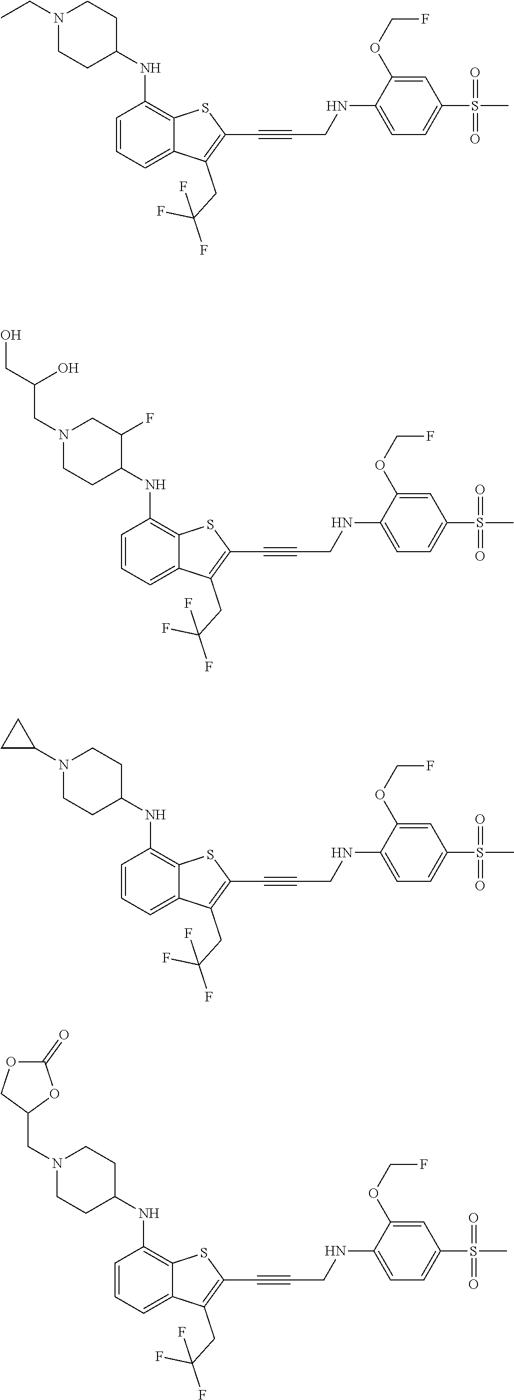 [SMILES: CCN1CCC(Nc2cccc3c(CC(F)(F)F)c(C#CCNc4ccc(S(C)(=O)=O)cc4OCF)sc23)CC1 ; CS(=O)(=O)c1ccc(NCC#Cc2sc3c(NC4CCN(CC(O)CO)CC4F)cccc3c2CC(F)(F)F)c(OCF)c1 ; CS(=O)(=O)c1ccc(NCC#Cc2sc3c(NC4CCN(C5CC5)CC4)cccc3c2CC(F)(F)F)c(OCF)c1 ; CS(=O)(=O)c1ccc(NCC#Cc2sc3c(NC4CCN(CC5COC(=O)O5)CC4)cccc3c2CC(F)(F)F)c(OCF)c1] 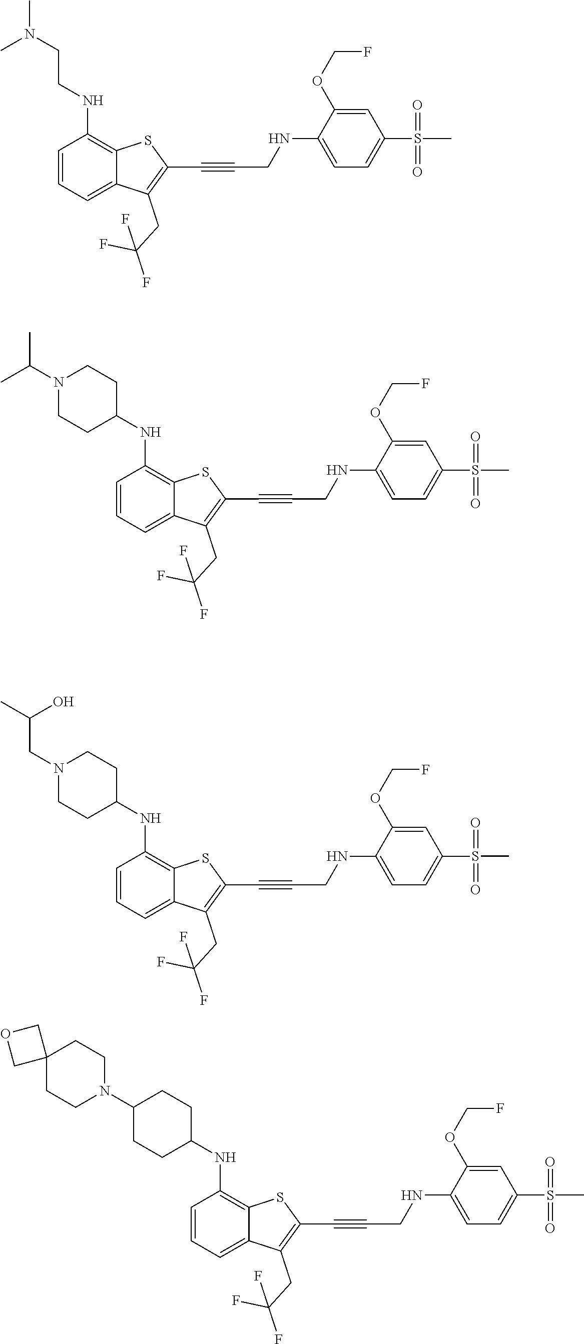 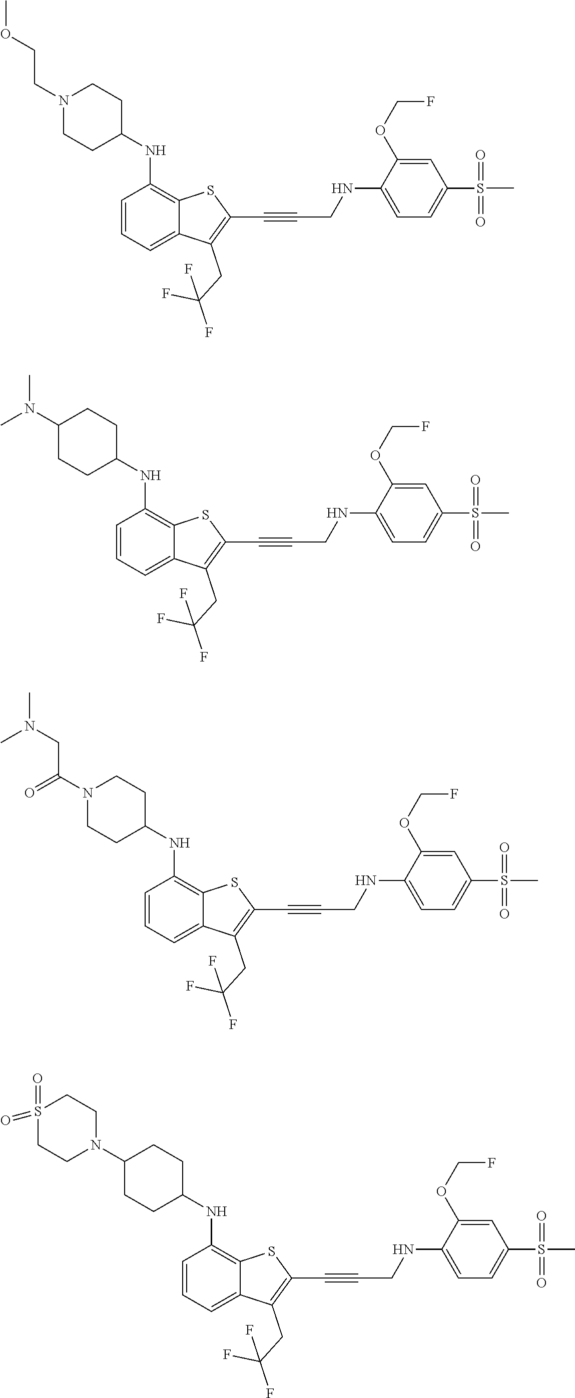 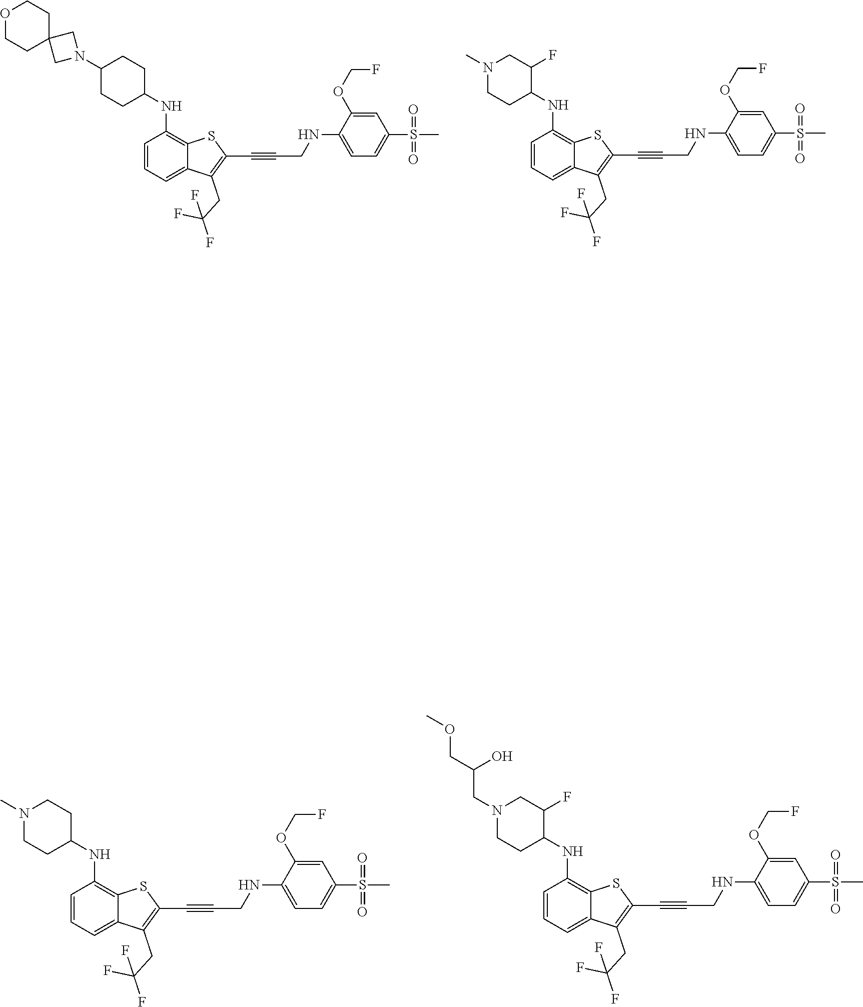 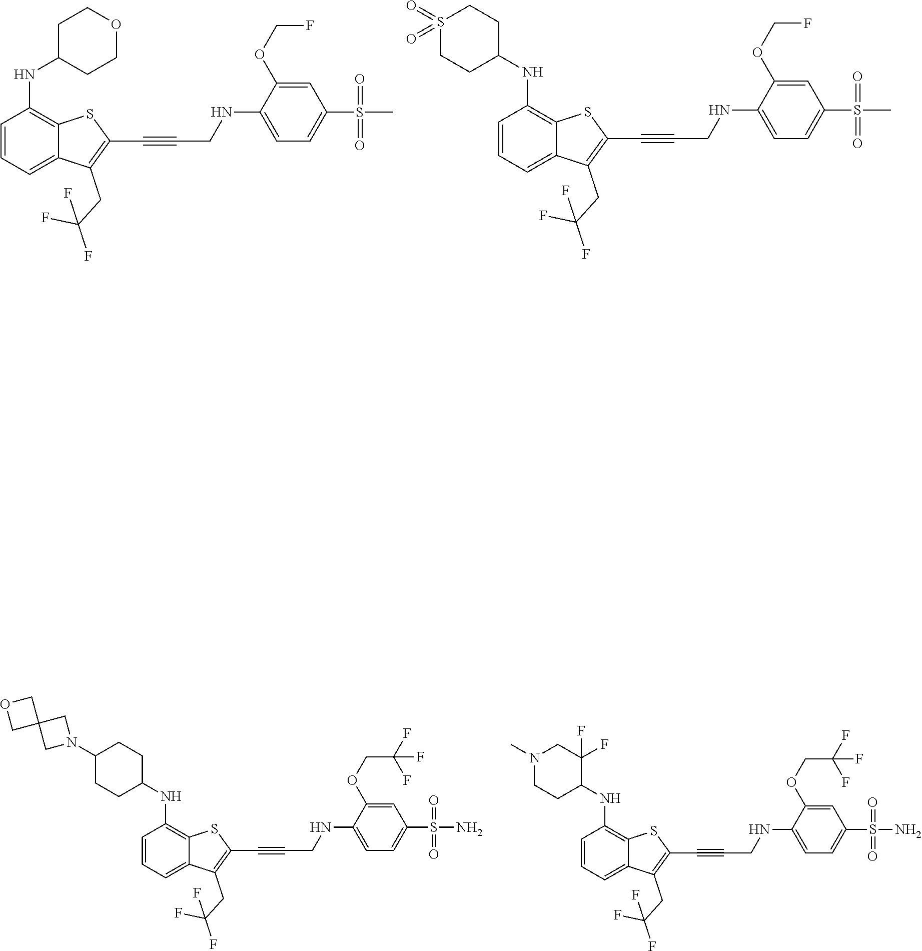 [SMILES: CS(=O)(=O)c1ccc(NCC#Cc2sc3c(NC4CCOCC4)cccc3c2CC(F)(F)F)c(OCF)c1 ; CS(=O)(=O)c1ccc(NCC#Cc2sc3c(NC4CCS(=O)(=O)CC4)cccc3c2CC(F)(F)F)c(OCF)c1 ; NS(=O)(=O)c1ccc(NCC#Cc2sc3c(NC4CCC(N5CC6(COC6)C5)CC4)cccc3c2CC(F)(F)F)c(OCC(F)(F)F)c1 ; CN1CCC(Nc2cccc3c(CC(F)(F)F)c(C#CCNc4ccc(S(N)(=O)=O)cc4OCC(F)(F)F)sc23)C(F)(F)C1] 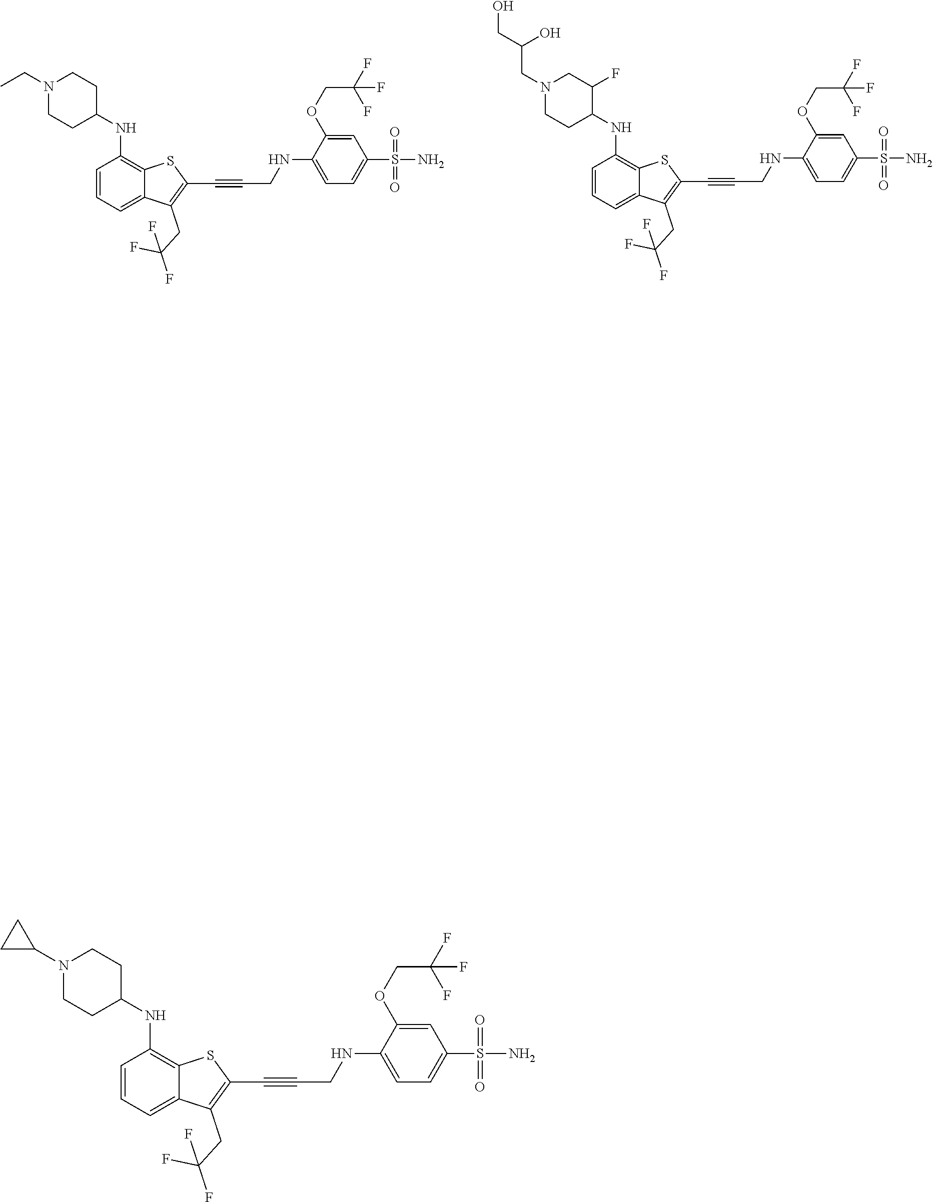 [SMILES: CCN1CCC(Nc2cccc3c(CC(F)(F)F)c(C#CCNc4ccc(S(N)(=O)=O)cc4OCC(F)(F)F)sc23)CC1 ; NS(=O)(=O)c1ccc(NCC#Cc2sc3c(NC4CCN(CC(O)CO)CC4F)cccc3c2CC(F)(F)F)c(OCC(F)(F)F)c1 ; NS(=O)(=O)c1ccc(NCC#Cc2sc3c(NC4CCN(C5CC5)CC4)cccc3c2CC(F)(F)F)c(OCC(F)(F)F)c1] 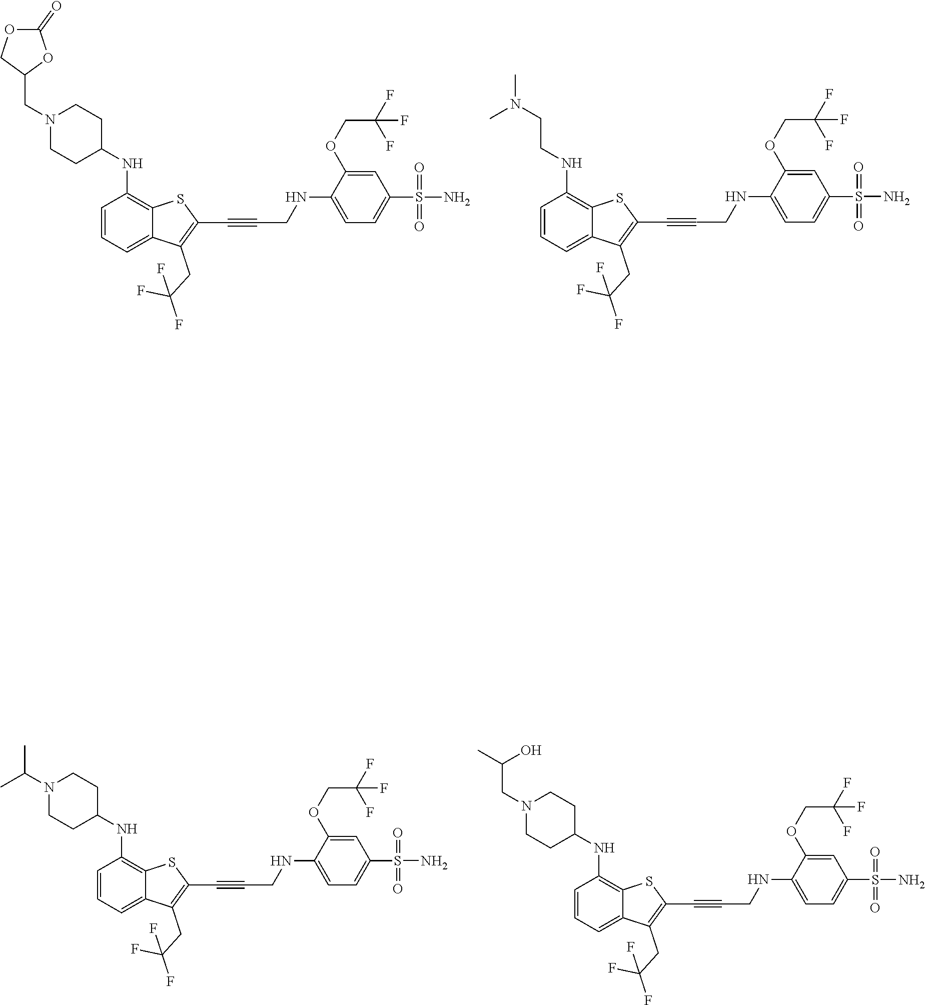 [SMILES: NS(=O)(=O)c1ccc(NCC#Cc2sc3c(NC4CCN(CC5COC(=O)O5)CC4)cccc3c2CC(F)(F)F)c(OCC(F)(F)F)c1 ; CN(C)CCNc1cccc2c(CC(F)(F)F)c(C#CCNc3ccc(S(N)(=O)=O)cc3OCC(F)(F)F)sc12 ; CC(C)N1CCC(Nc2cccc3c(CC(F)(F)F)c(C#CCNc4ccc(S(N)(=O)=O)cc4OCC(F)(F)F)sc23)CC1 ; CC(O)CN1CCC(Nc2cccc3c(CC(F)(F)F)c(C#CCNc4ccc(S(N)(=O)=O)cc4OCC(F)(F)F)sc23)CC1] 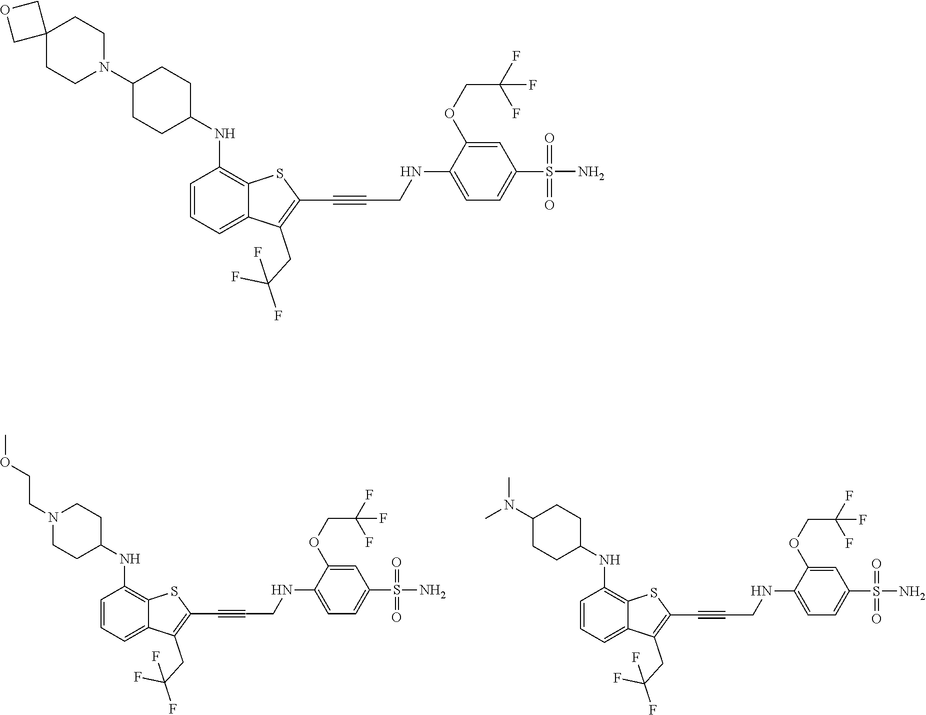 [SMILES: NS(=O)(=O)c1ccc(NCC#Cc2sc3c(NC4CCC(N5CCC6(CC5)COC6)CC4)cccc3c2CC(F)(F)F)c(OCC(F)(F)F)c1 ; COCCN1CCC(Nc2cccc3c(CC(F)(F)F)c(C#CCNc4ccc(S(N)(=O)=O)cc4OCC(F)(F)F)sc23)CC1 ; CN(C)C1CCC(Nc2cccc3c(CC(F)(F)F)c(C#CCNc4ccc(S(N)(=O)=O)cc4OCC(F)(F)F)sc23)CC1] 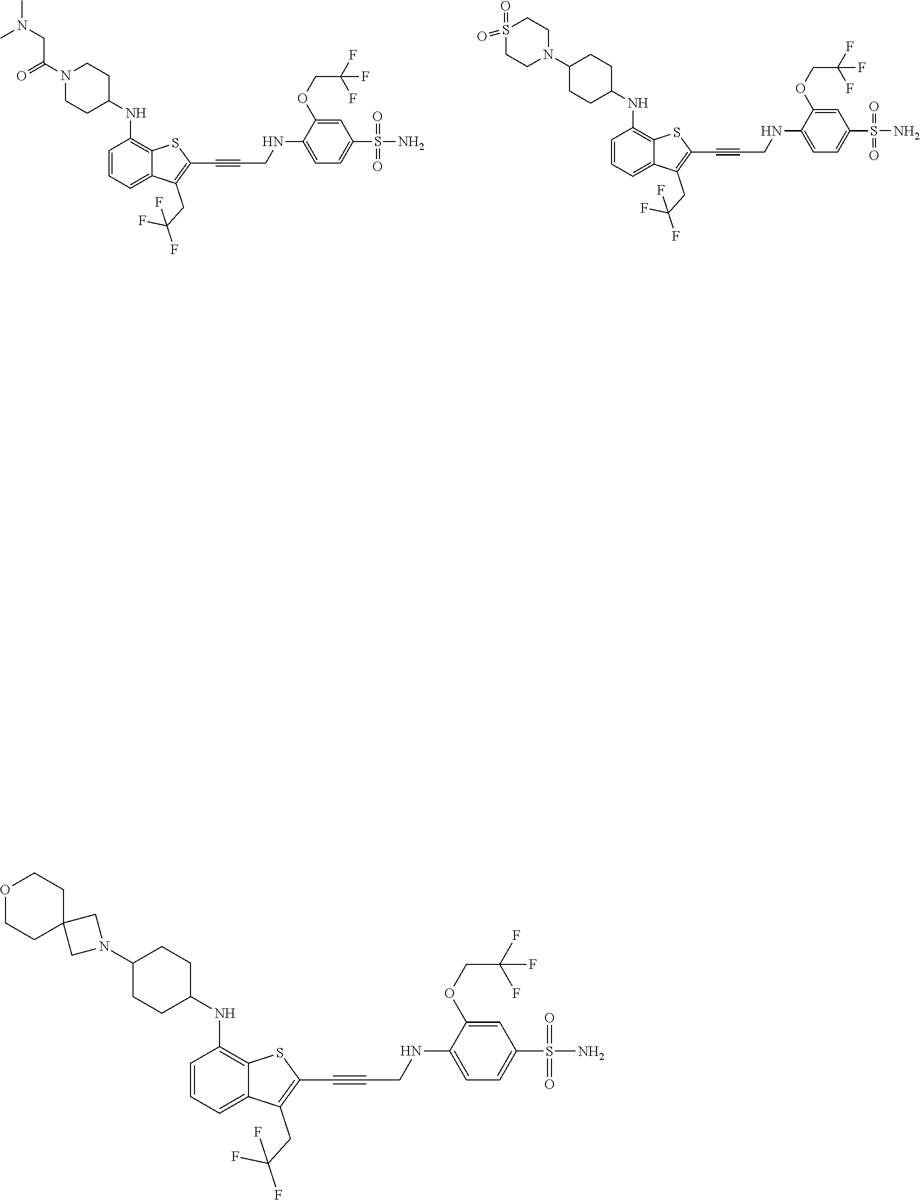 [SMILES: CN(C)CC(=O)N1CCC(Nc2cccc3c(CC(F)(F)F)c(C#CCNc4ccc(S(N)(=O)=O)cc4OCC(F)(F)F)sc23)CC1 ; NS(=O)(=O)c1ccc(NCC#Cc2sc3c(NC4CCC(N5CCS(=O)(=O)CC5)CC4)cccc3c2CC(F)(F)F)c(OCC(F)(F)F)c1 ; NS(=O)(=O)c1ccc(NCC#Cc2sc3c(NC4CCC(N5CC6(CCOCC6)C5)CC4)cccc3c2CC(F)(F)F)c(OCC(F)(F)F)c1] 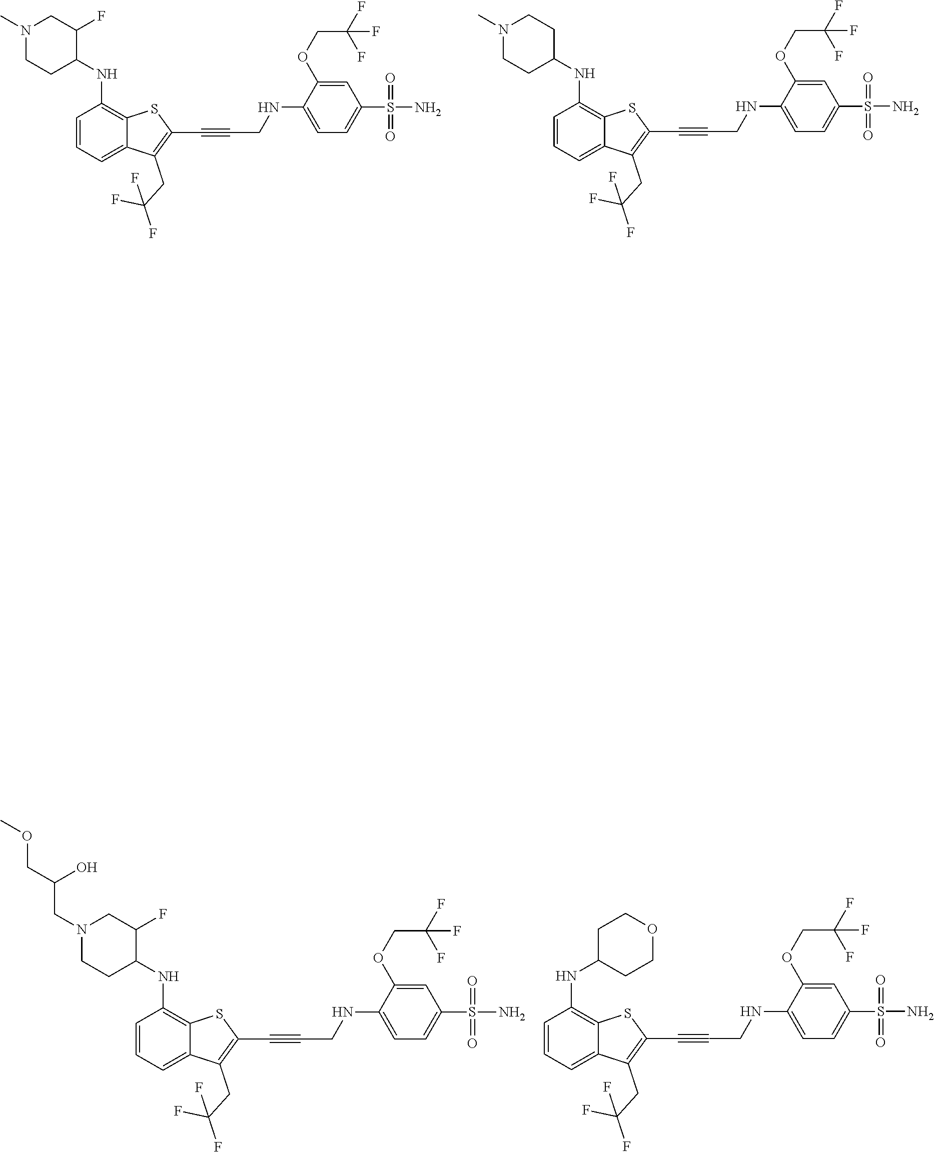 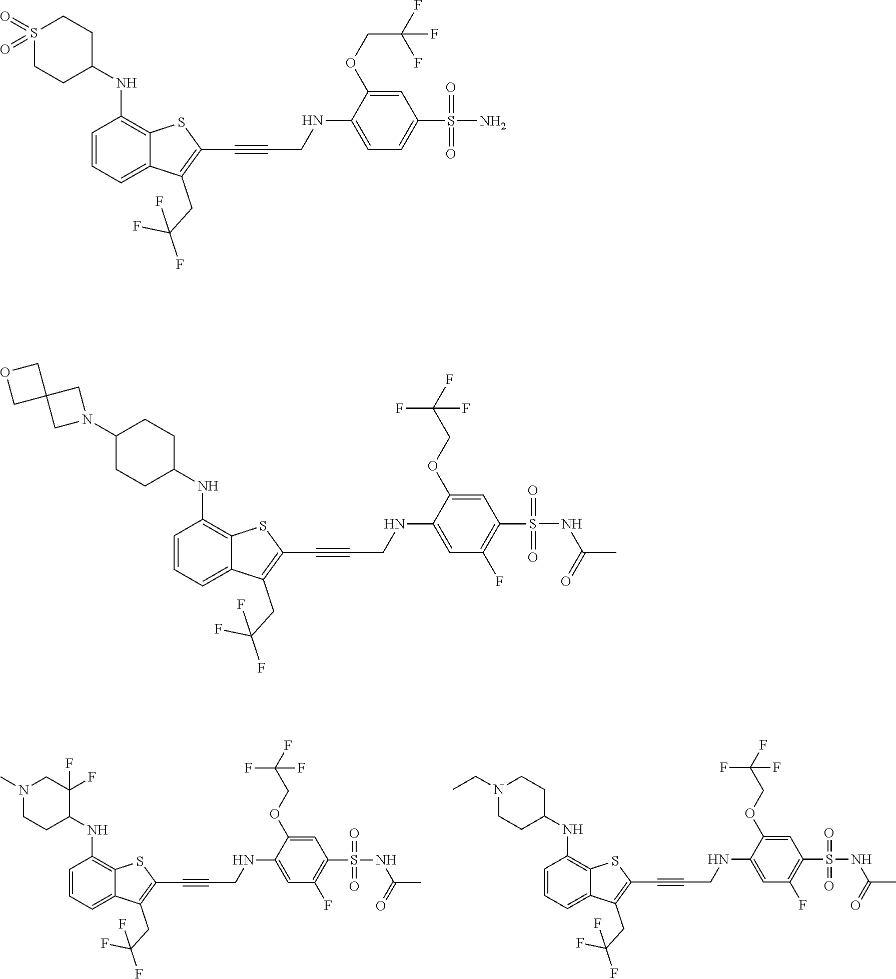 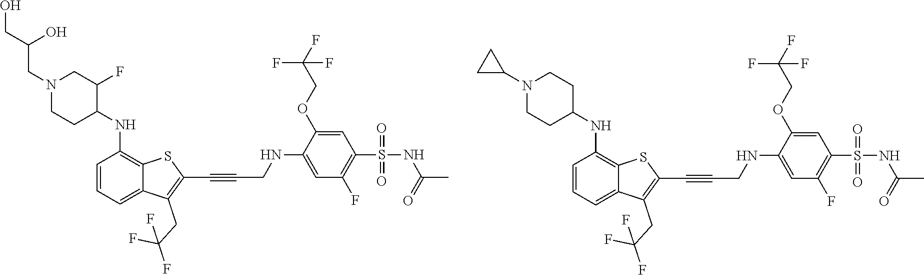 [SMILES: CC(=O)NS(=O)(=O)c1cc(OCC(F)(F)F)c(NCC#Cc2sc3c(NC4CCN(CC(O)CO)CC4F)cccc3c2CC(F)(F)F)cc1F ; CC(=O)NS(=O)(=O)c1cc(OCC(F)(F)F)c(NCC#Cc2sc3c(NC4CCN(C5CC5)CC4)cccc3c2CC(F)(F)F)cc1F] 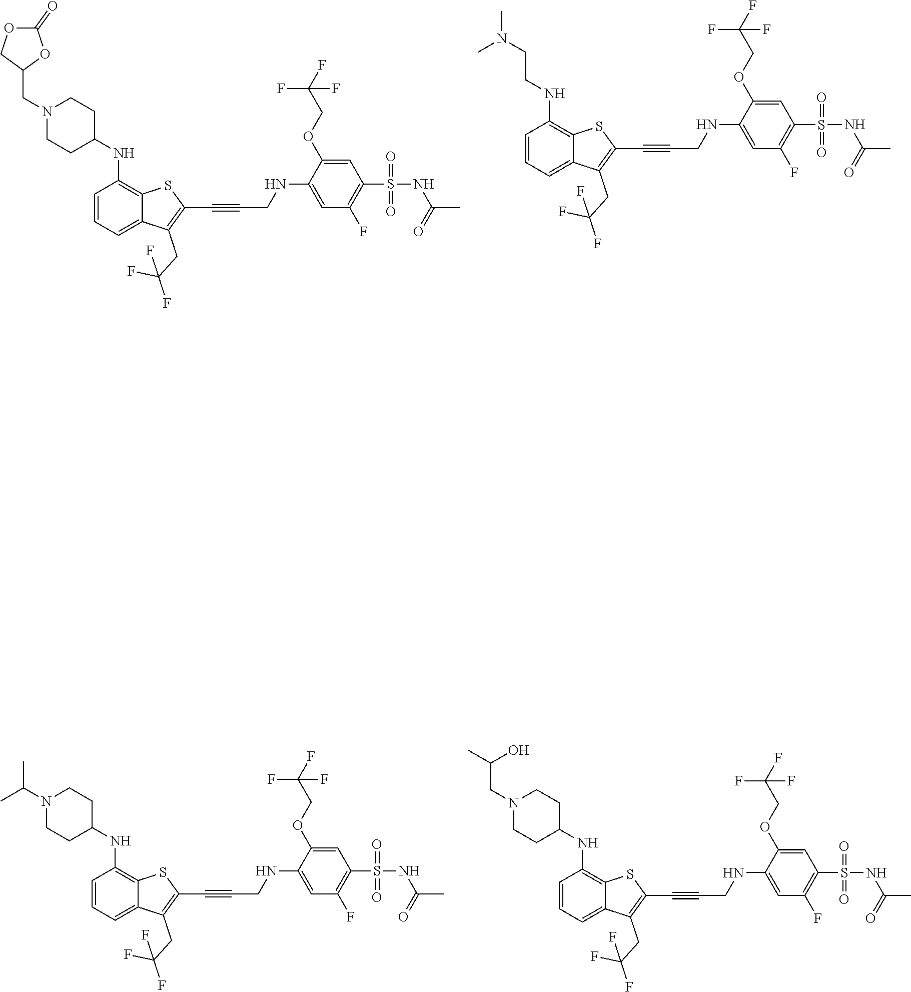 [SMILES: CC(=O)NS(=O)(=O)c1cc(OCC(F)(F)F)c(NCC#Cc2sc3c(NC4CCN(CC5COC(=O)O5)CC4)cccc3c2CC(F)(F)F)cc1F ; CC(=O)NS(=O)(=O)c1cc(OCC(F)(F)F)c(NCC#Cc2sc3c(NCCN(C)C)cccc3c2CC(F)(F)F)cc1F ; CC(=O)NS(=O)(=O)c1cc(OCC(F)(F)F)c(NCC#Cc2sc3c(NC4CCN(C(C)C)CC4)cccc3c2CC(F)(F)F)cc1F ; CC(=O)NS(=O)(=O)c1cc(OCC(F)(F)F)c(NCC#Cc2sc3c(NC4CCN(CC(C)O)CC4)cccc3c2CC(F)(F)F)cc1F] 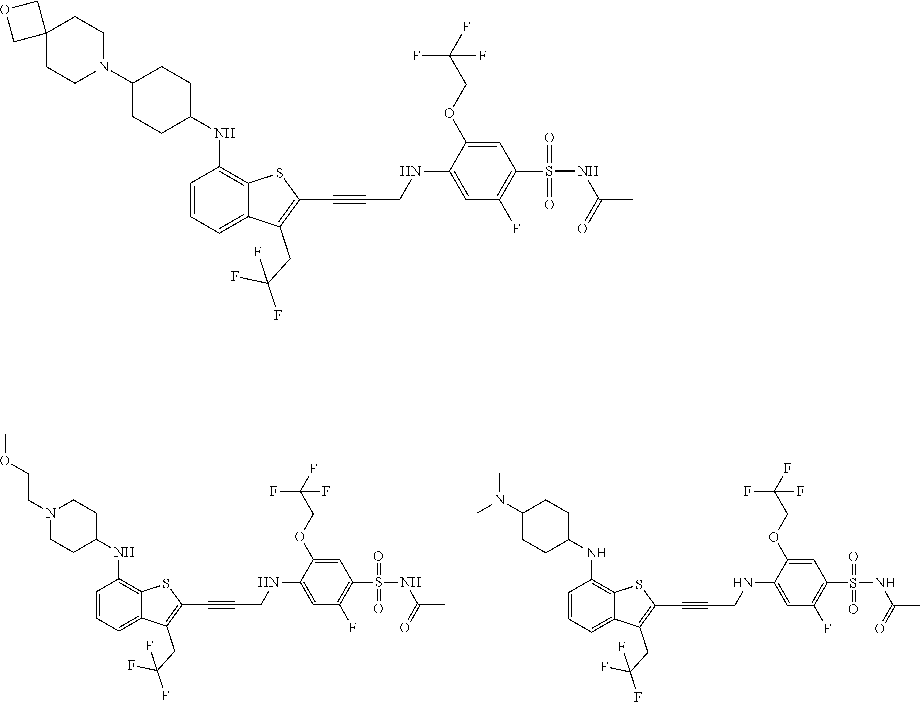 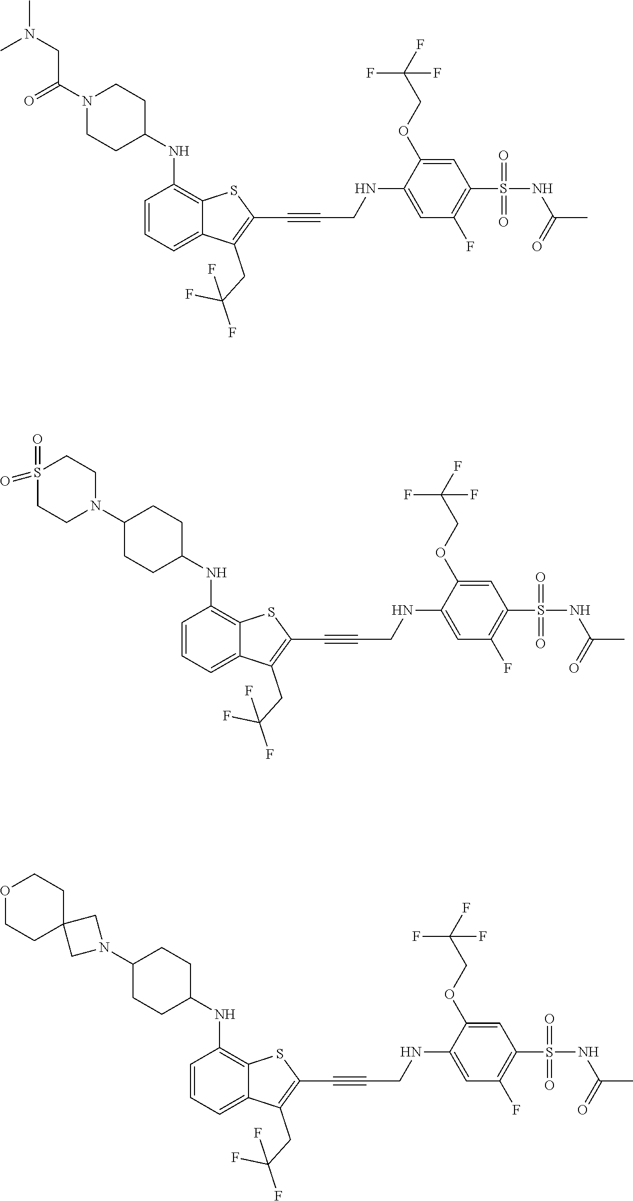 [SMILES: CC(=O)NS(=O)(=O)c1cc(OCC(F)(F)F)c(NCC#Cc2sc3c(NC4CCN(C(=O)CN(C)C)CC4)cccc3c2CC(F)(F)F)cc1F ; CC(=O)NS(=O)(=O)c1cc(OCC(F)(F)F)c(NCC#Cc2sc3c(NC4CCC(N5CCS(=O)(=O)CC5)CC4)cccc3c2CC(F)(F)F)cc1F ; CC(=O)NS(=O)(=O)c1cc(OCC(F)(F)F)c(NCC#Cc2sc3c(NC4CCC(N5CC6(CCOCC6)C5)CC4)cccc3c2CC(F)(F)F)cc1F] 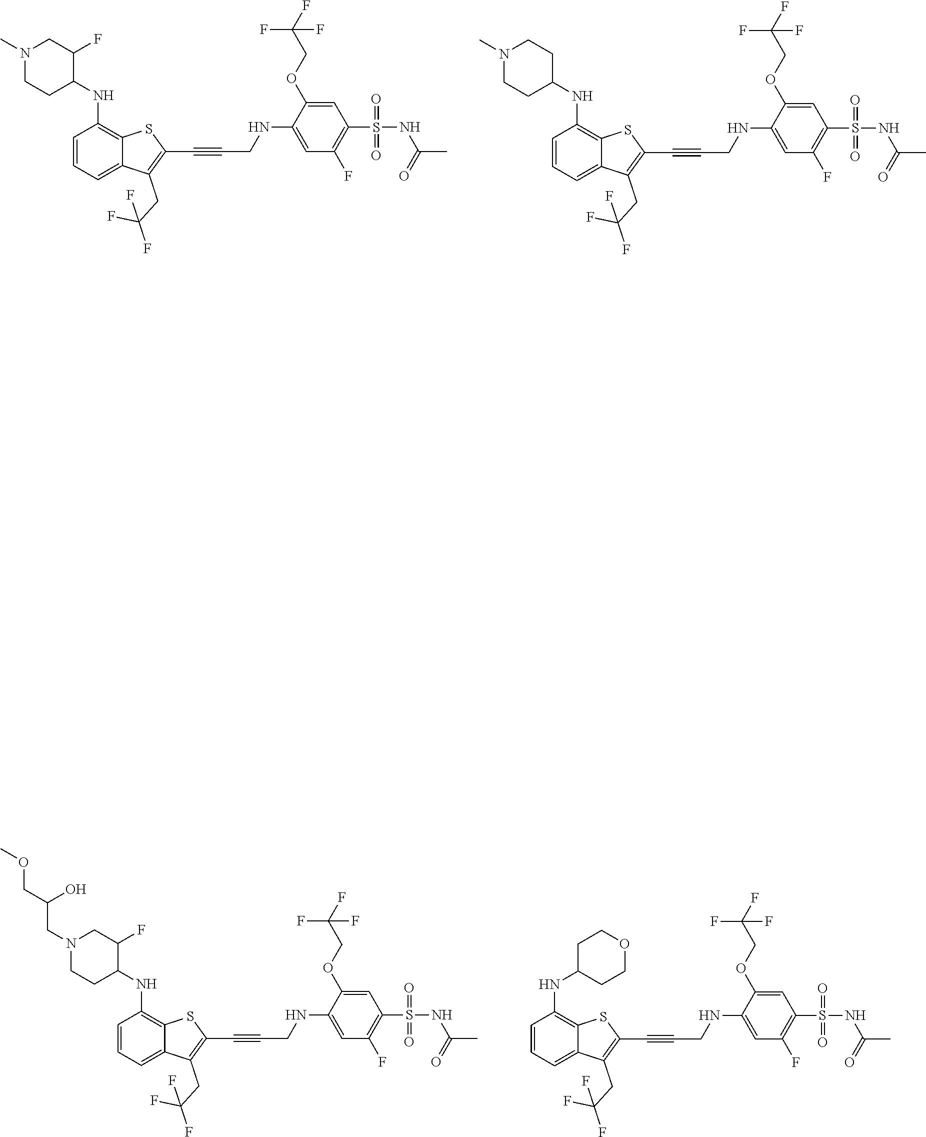 [SMILES: CC(=O)NS(=O)(=O)c1cc(OCC(F)(F)F)c(NCC#Cc2sc3c(NC4CCN(C)CC4F)cccc3c2CC(F)(F)F)cc1F ; CC(=O)NS(=O)(=O)c1cc(OCC(F)(F)F)c(NCC#Cc2sc3c(NC4CCN(C)CC4)cccc3c2CC(F)(F)F)cc1F ; COCC(O)CN1CCC(Nc2cccc3c(CC(F)(F)F)c(C#CCNc4cc(F)c(S(=O)(=O)NC(C)=O)cc4OCC(F)(F)F)sc23)C(F)C1 ; CC(=O)NS(=O)(=O)c1cc(OCC(F)(F)F)c(NCC#Cc2sc3c(NC4CCOCC4)cccc3c2CC(F)(F)F)cc1F] 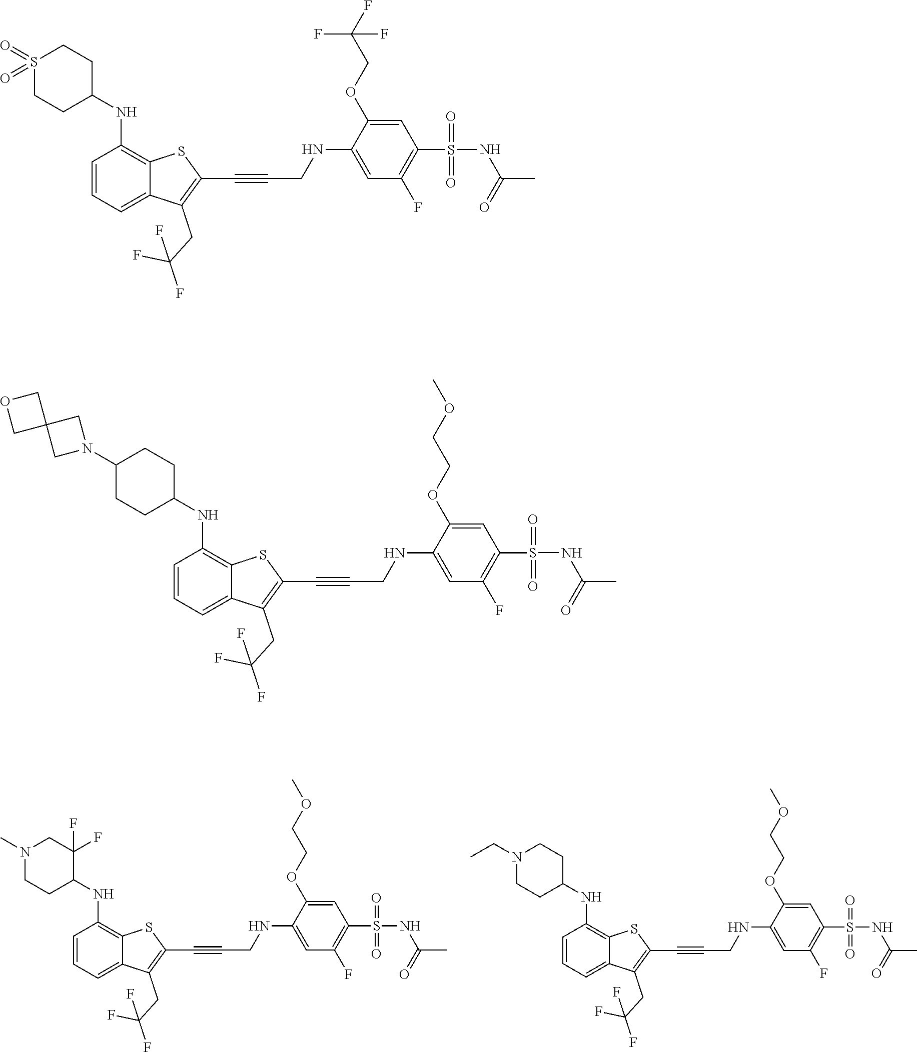 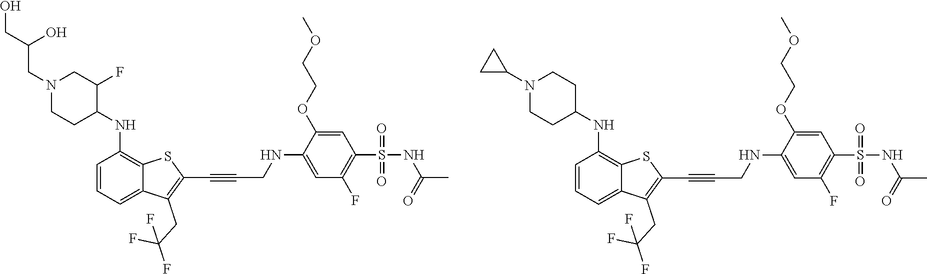 [SMILES: COCCOc1cc(S(=O)(=O)NC(C)=O)c(F)cc1NCC#Cc1sc2c(NC3CCN(CC(O)CO)CC3F)cccc2c1CC(F)(F)F ; COCCOc1cc(S(=O)(=O)NC(C)=O)c(F)cc1NCC#Cc1sc2c(NC3CCN(C4CC4)CC3)cccc2c1CC(F)(F)F] 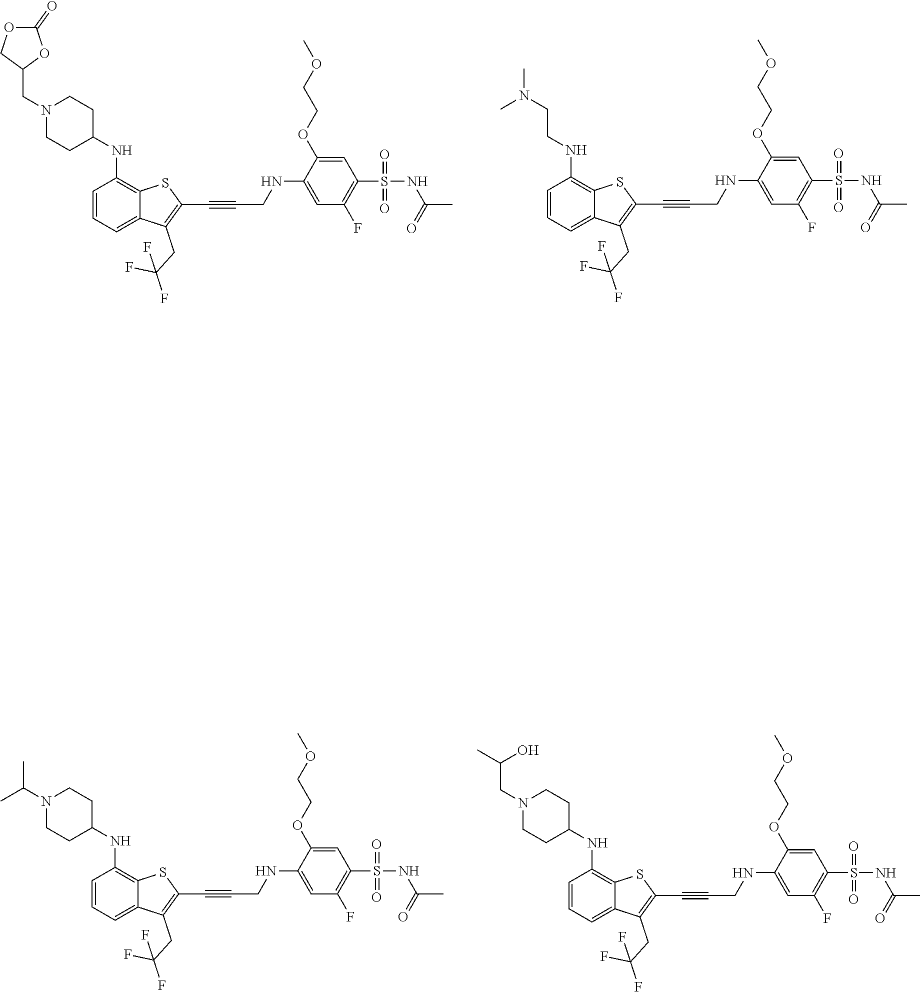 [SMILES: COCCOc1cc(S(=O)(=O)NC(C)=O)c(F)cc1NCC#Cc1sc2c(NC3CCN(CC4COC(=O)O4)CC3)cccc2c1CC(F)(F)F ; COCCOc1cc(S(=O)(=O)NC(C)=O)c(F)cc1NCC#Cc1sc2c(NCCN(C)C)cccc2c1CC(F)(F)F ; COCCOc1cc(S(=O)(=O)NC(C)=O)c(F)cc1NCC#Cc1sc2c(NC3CCN(C(C)C)CC3)cccc2c1CC(F)(F)F ; COCCOc1cc(S(=O)(=O)NC(C)=O)c(F)cc1NCC#Cc1sc2c(NC3CCN(CC(C)O)CC3)cccc2c1CC(F)(F)F] 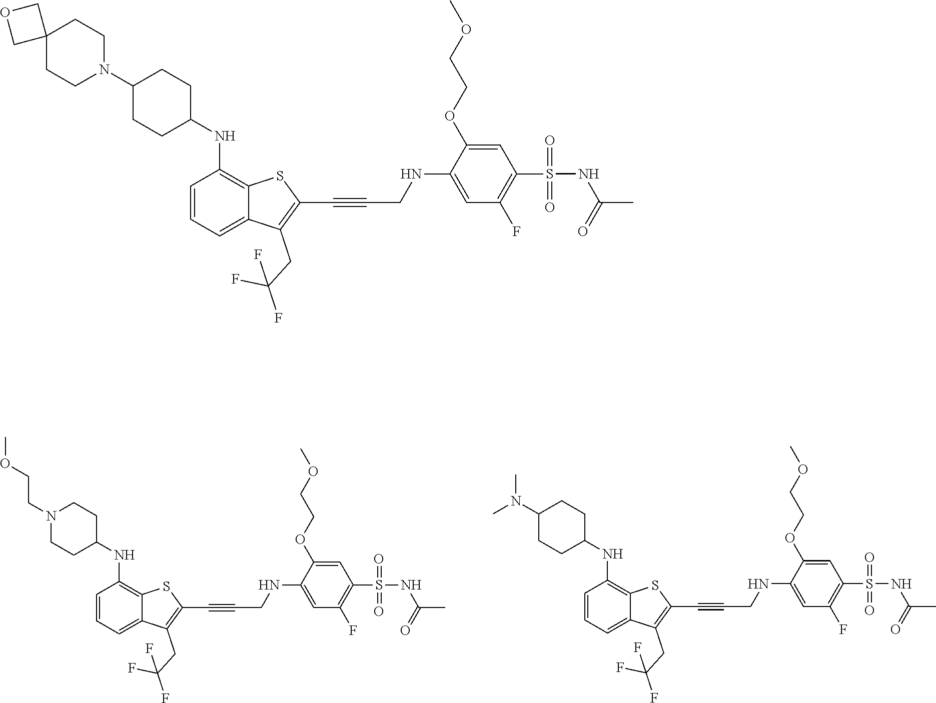 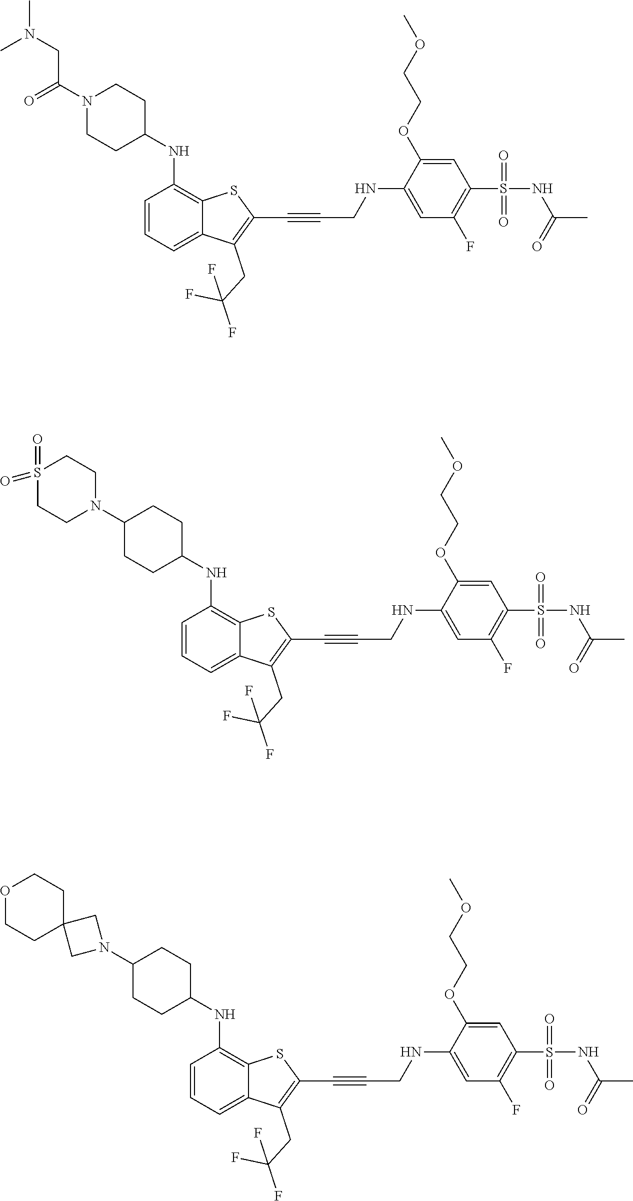 [SMILES: COCCOc1cc(S(=O)(=O)NC(C)=O)c(F)cc1NCC#Cc1sc2c(NC3CCN(C(=O)CN(C)C)CC3)cccc2c1CC(F)(F)F ; COCCOc1cc(S(=O)(=O)NC(C)=O)c(F)cc1NCC#Cc1sc2c(NC3CCC(N4CCS(=O)(=O)CC4)CC3)cccc2c1CC(F)(F)F ; COCCOc1cc(S(=O)(=O)NC(C)=O)c(F)cc1NCC#Cc1sc2c(NC3CCC(N4CC5(CCOCC5)C4)CC3)cccc2c1CC(F)(F)F] 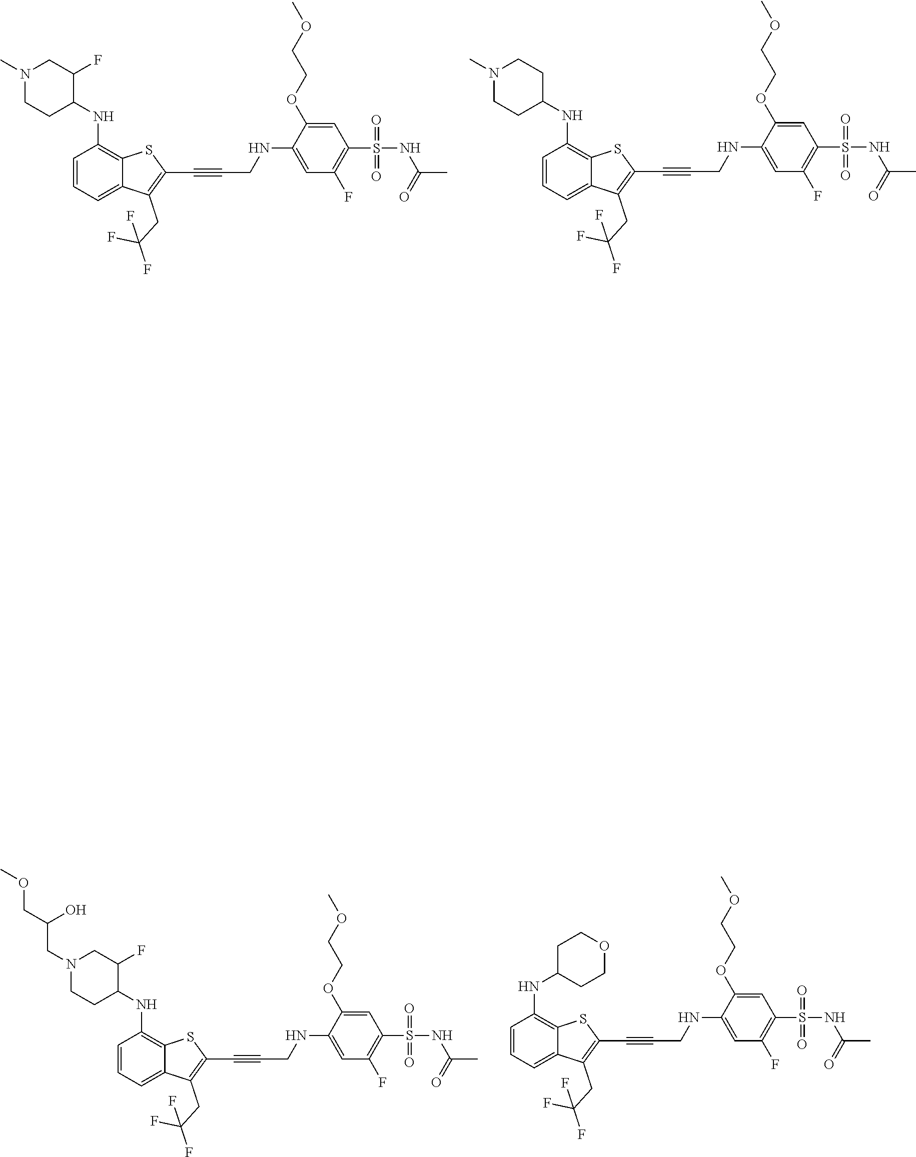 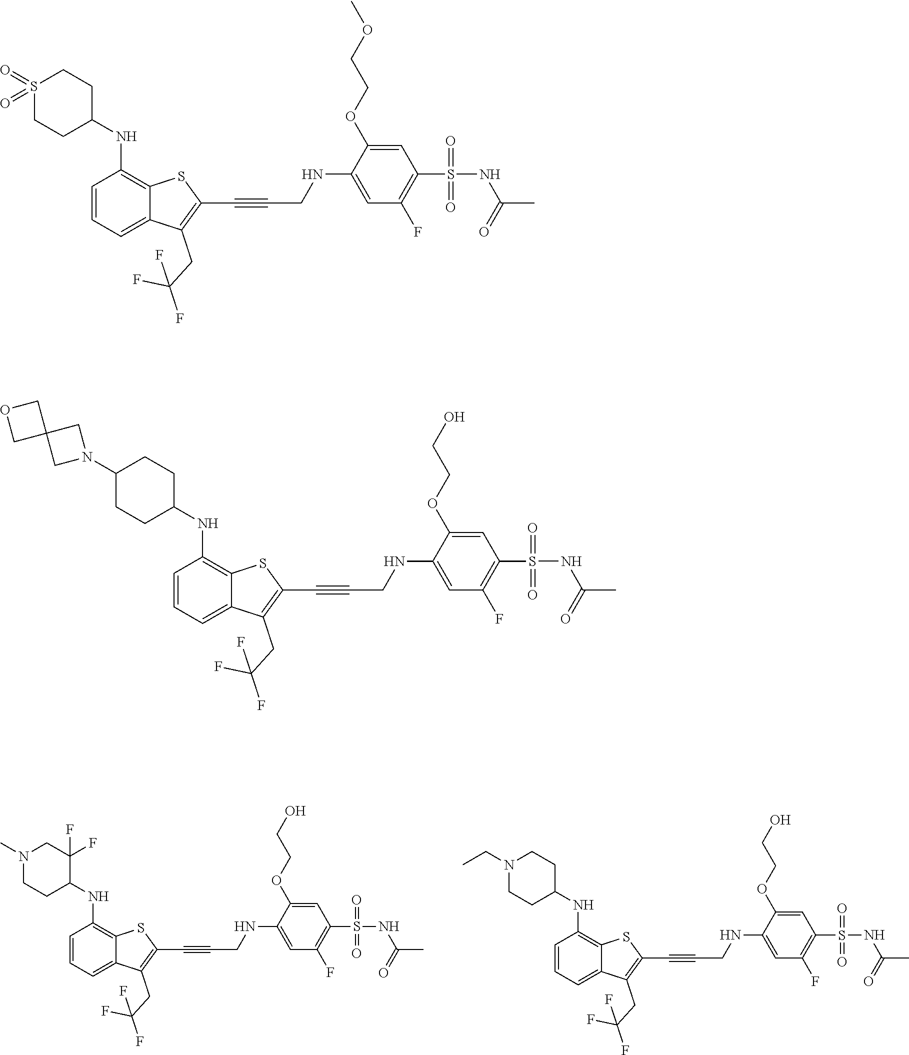 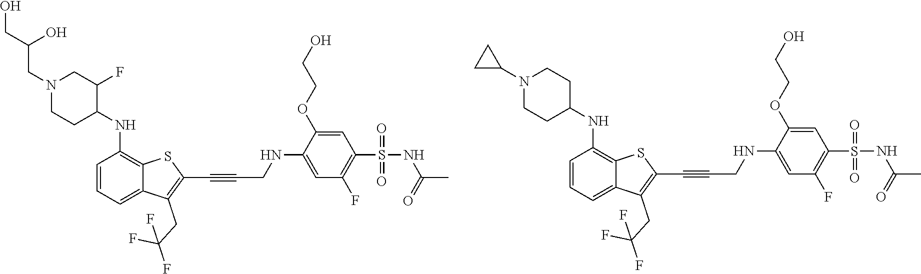 [SMILES: CC(=O)NS(=O)(=O)c1cc(OCCO)c(NCC#Cc2sc3c(NC4CCN(CC(O)CO)CC4F)cccc3c2CC(F)(F)F)cc1F ; CC(=O)NS(=O)(=O)c1cc(OCCO)c(NCC#Cc2sc3c(NC4CCN(C5CC5)CC4)cccc3c2CC(F)(F)F)cc1F] 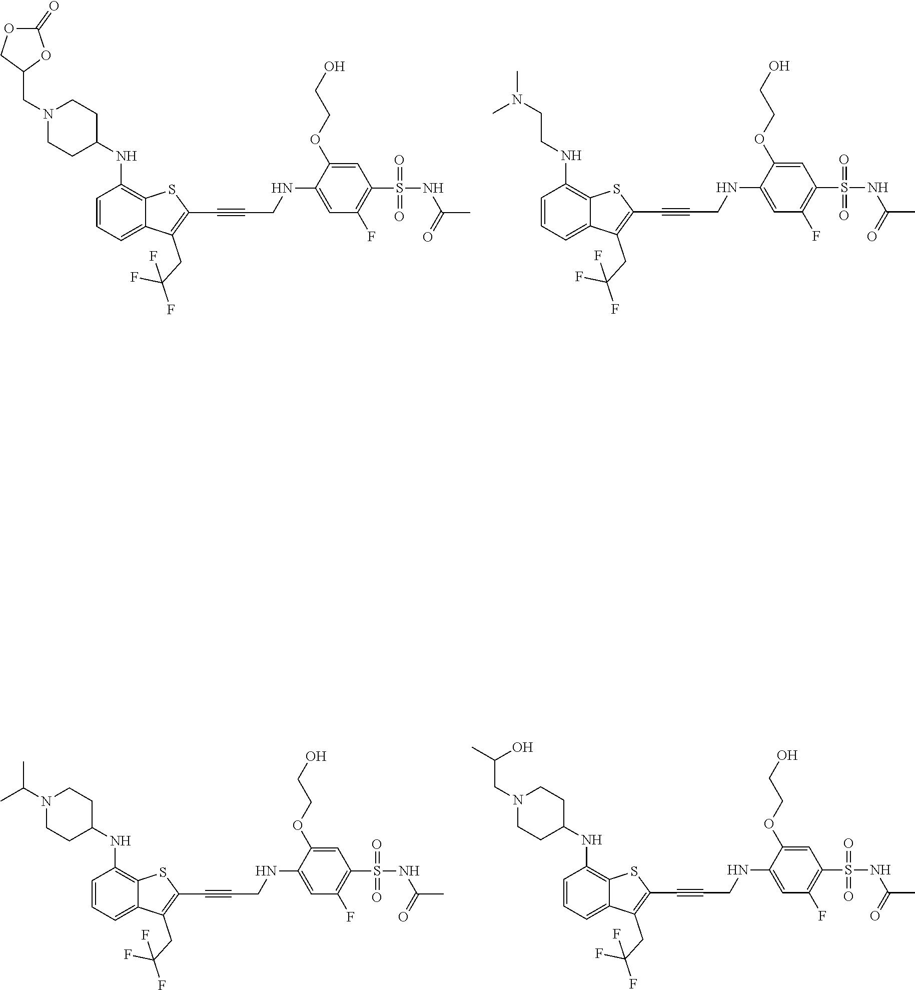 [SMILES: CC(=O)NS(=O)(=O)c1cc(OCCO)c(NCC#Cc2sc3c(NC4CCN(CC5COC(=O)O5)CC4)cccc3c2CC(F)(F)F)cc1F ; CC(=O)NS(=O)(=O)c1cc(OCCO)c(NCC#Cc2sc3c(NCCN(C)C)cccc3c2CC(F)(F)F)cc1F ; CC(=O)NS(=O)(=O)c1cc(OCCO)c(NCC#Cc2sc3c(NC4CCN(C(C)C)CC4)cccc3c2CC(F)(F)F)cc1F ; CC(=O)NS(=O)(=O)c1cc(OCCO)c(NCC#Cc2sc3c(NC4CCN(CC(C)O)CC4)cccc3c2CC(F)(F)F)cc1F] 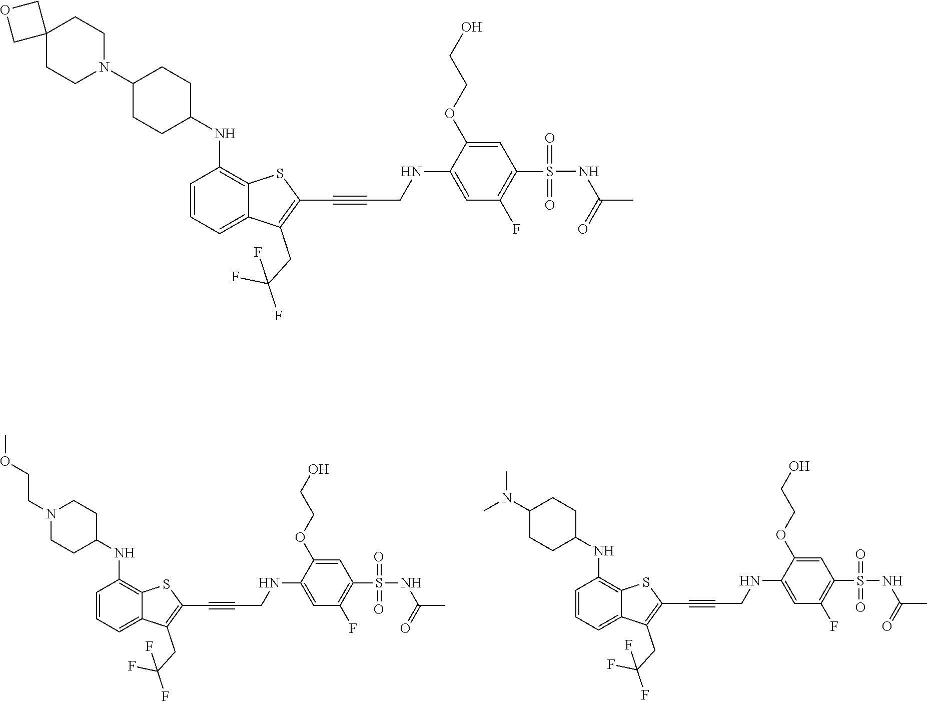 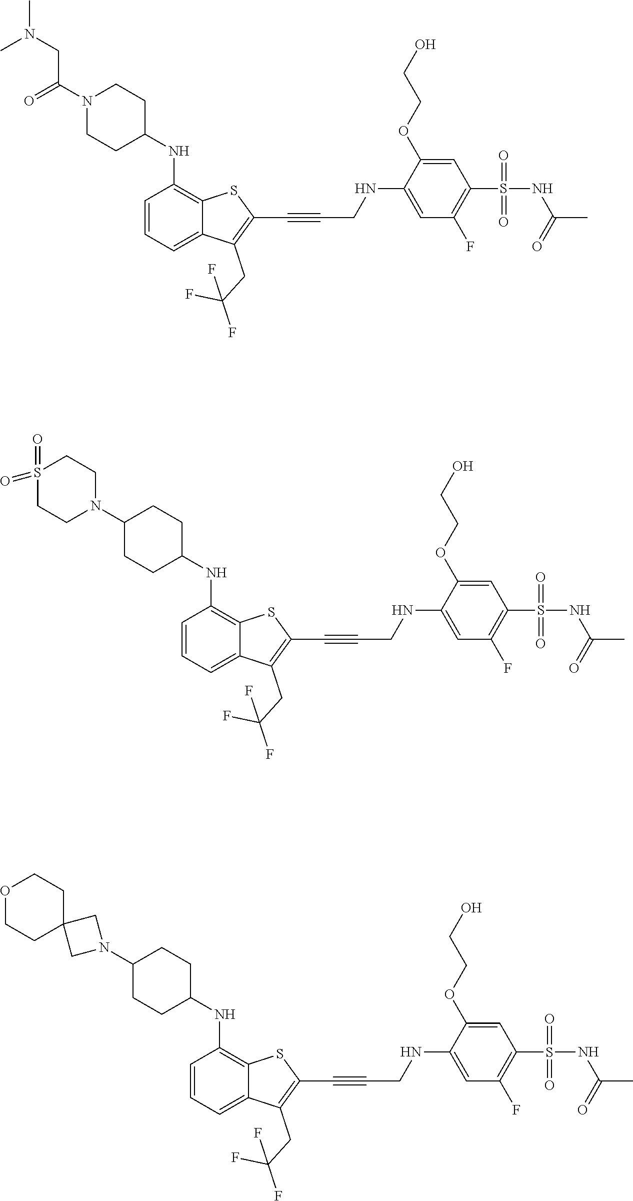 [SMILES: CC(=O)NS(=O)(=O)c1cc(OCCO)c(NCC#Cc2sc3c(NC4CCN(C(=O)CN(C)C)CC4)cccc3c2CC(F)(F)F)cc1F ; CC(=O)NS(=O)(=O)c1cc(OCCO)c(NCC#Cc2sc3c(NC4CCC(N5CCS(=O)(=O)CC5)CC4)cccc3c2CC(F)(F)F)cc1F ; CC(=O)NS(=O)(=O)c1cc(OCCO)c(NCC#Cc2sc3c(NC4CCC(N5CC6(CCOCC6)C5)CC4)cccc3c2CC(F)(F)F)cc1F] 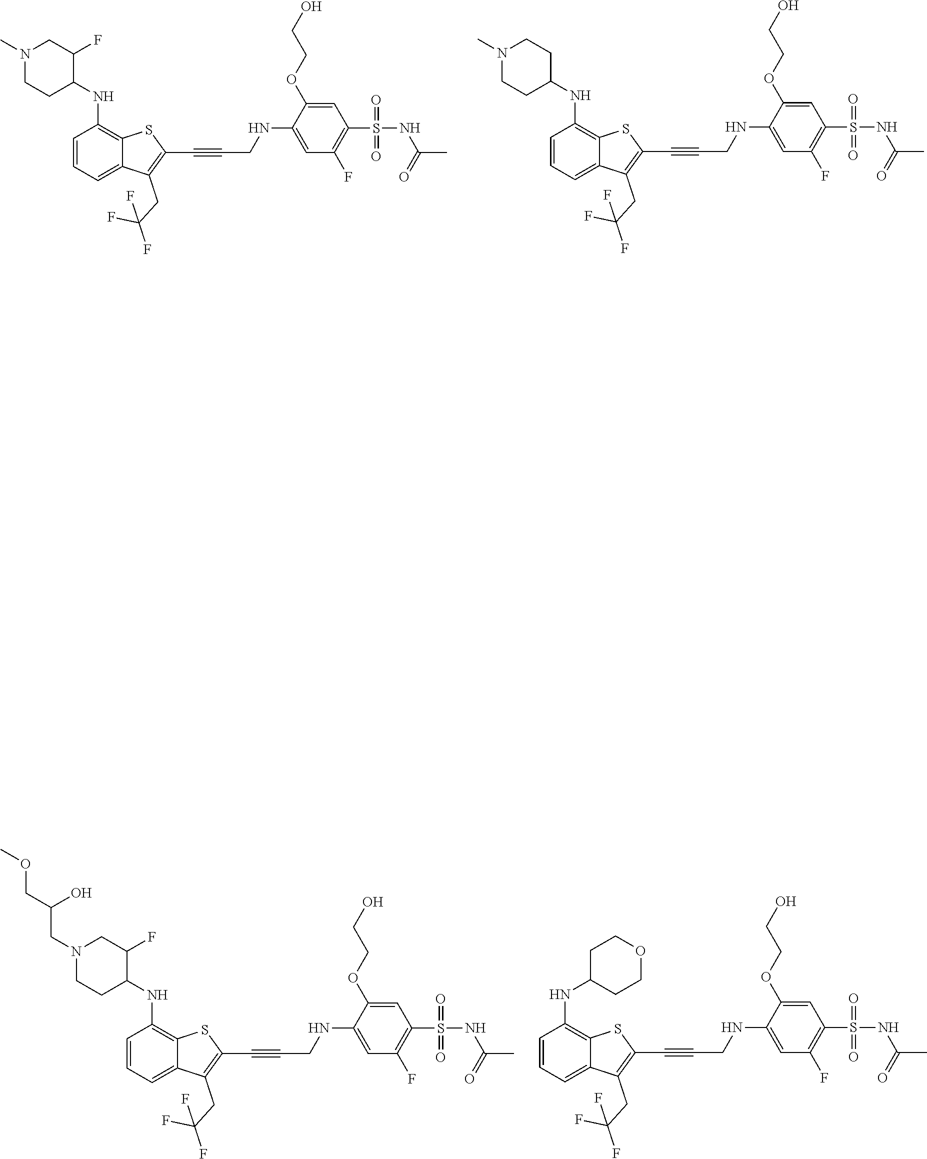 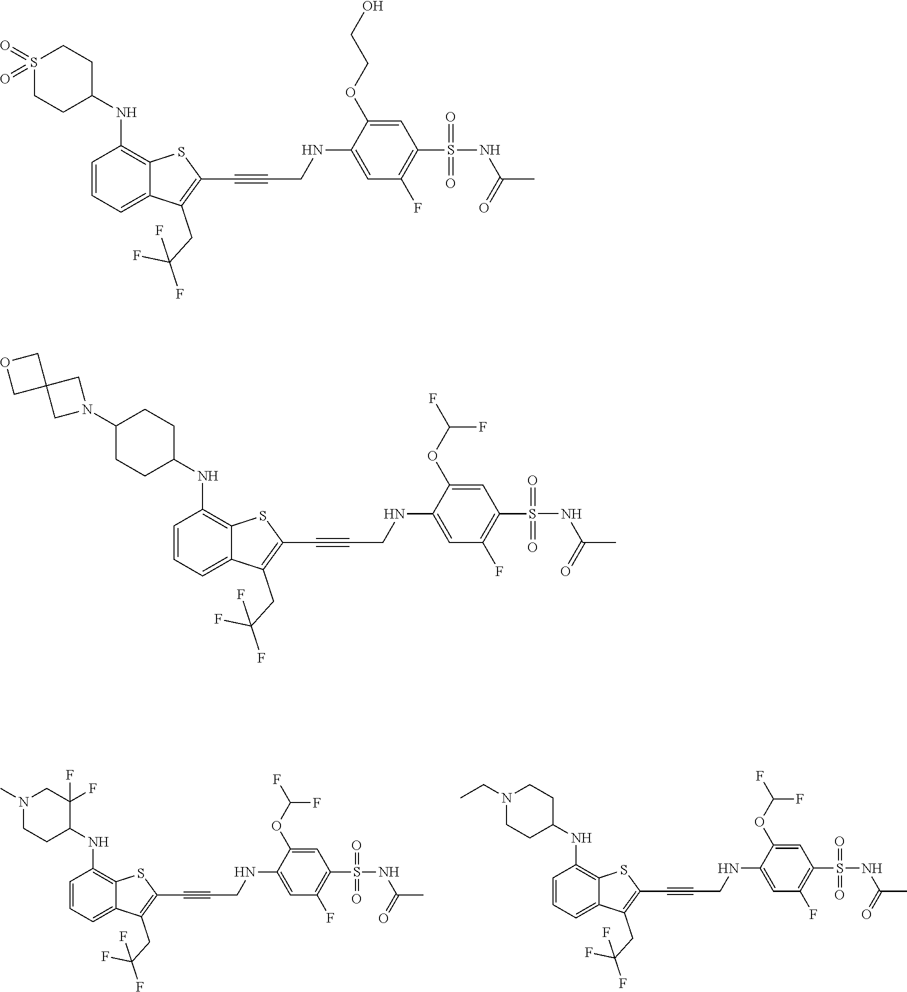 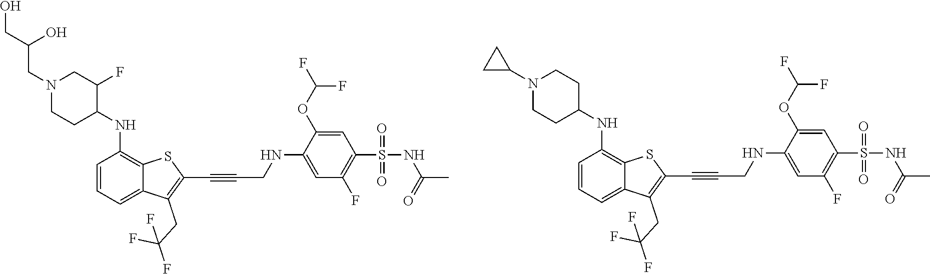 [SMILES: CC(=O)NS(=O)(=O)c1cc(OC(F)F)c(NCC#Cc2sc3c(NC4CCN(CC(O)CO)CC4F)cccc3c2CC(F)(F)F)cc1F ; CC(=O)NS(=O)(=O)c1cc(OC(F)F)c(NCC#Cc2sc3c(NC4CCN(C5CC5)CC4)cccc3c2CC(F)(F)F)cc1F] 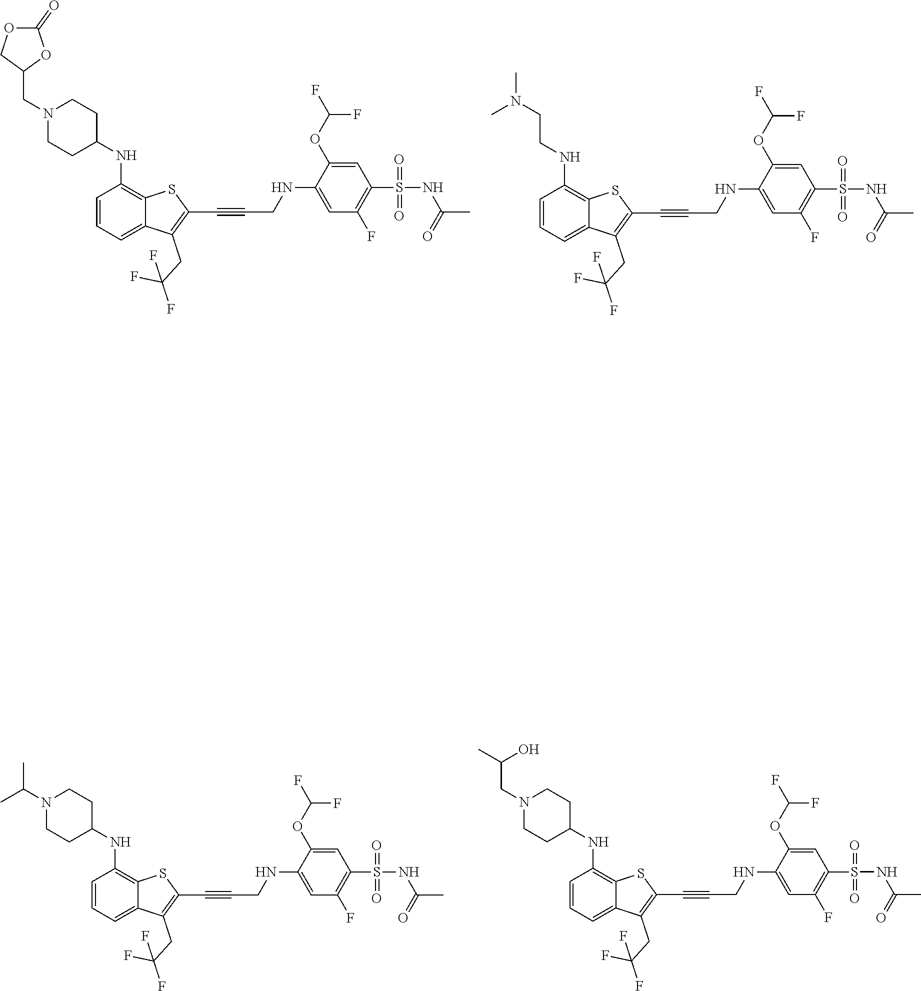 [SMILES: CC(=O)NS(=O)(=O)c1cc(OC(F)F)c(NCC#Cc2sc3c(NC4CCN(CC5COC(=O)O5)CC4)cccc3c2CC(F)(F)F)cc1F ; CC(=O)NS(=O)(=O)c1cc(OC(F)F)c(NCC#Cc2sc3c(NCCN(C)C)cccc3c2CC(F)(F)F)cc1F ; CC(=O)NS(=O)(=O)c1cc(OC(F)F)c(NCC#Cc2sc3c(NC4CCN(C(C)C)CC4)cccc3c2CC(F)(F)F)cc1F ; CC(=O)NS(=O)(=O)c1cc(OC(F)F)c(NCC#Cc2sc3c(NC4CCN(CC(C)O)CC4)cccc3c2CC(F)(F)F)cc1F] 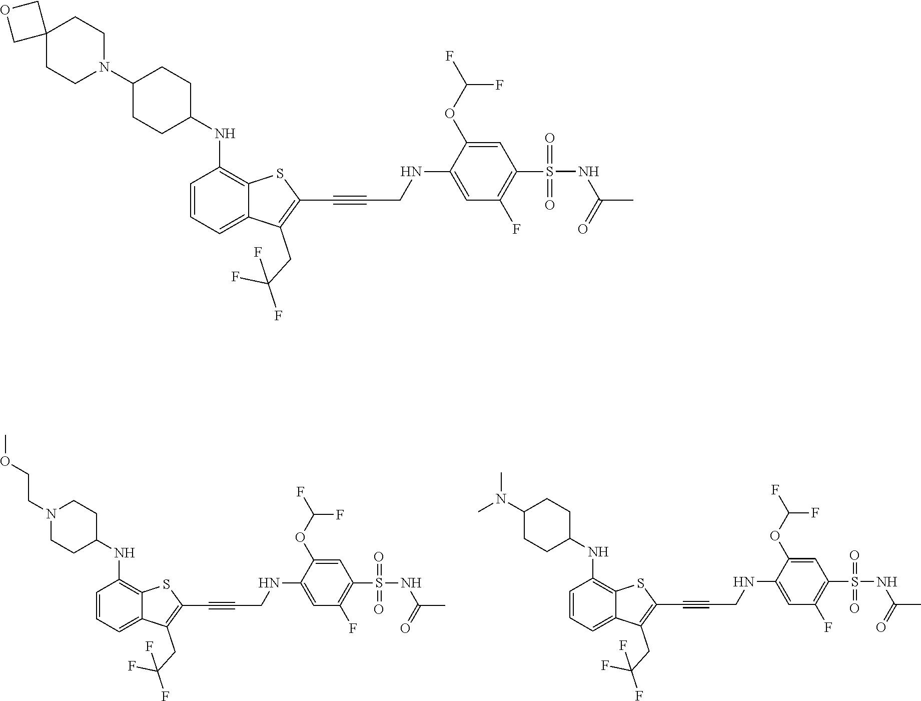 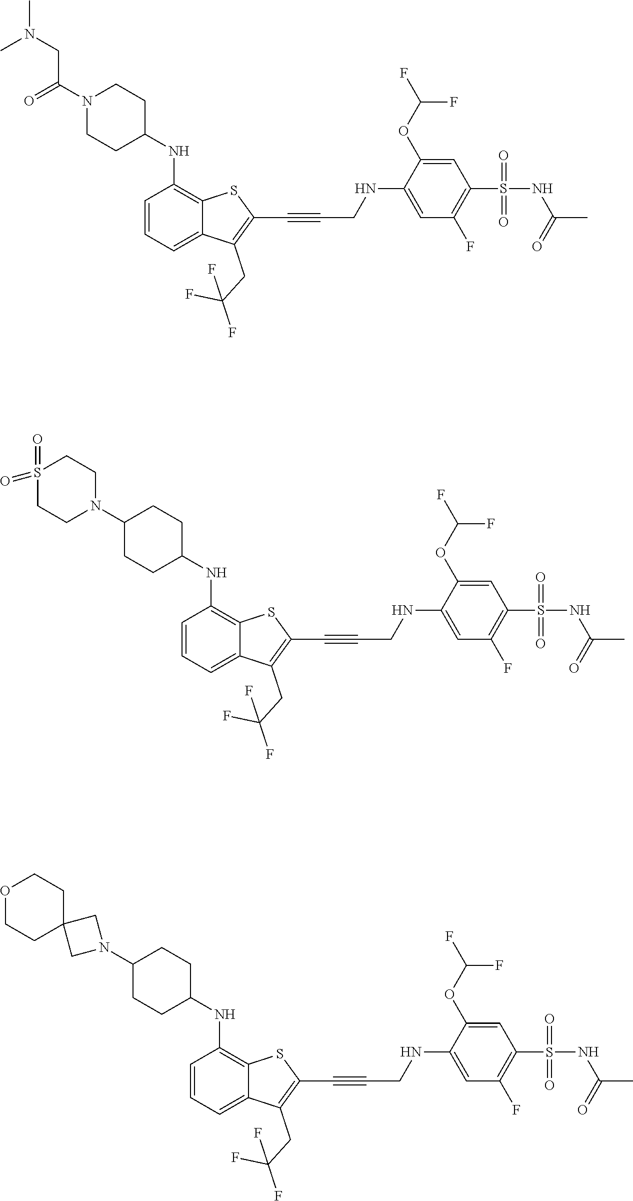 [SMILES: CC(=O)NS(=O)(=O)c1cc(OC(F)F)c(NCC#Cc2sc3c(NC4CCN(C(=O)CN(C)C)CC4)cccc3c2CC(F)(F)F)cc1F ; CC(=O)NS(=O)(=O)c1cc(OC(F)F)c(NCC#Cc2sc3c(NC4CCC(N5CCS(=O)(=O)CC5)CC4)cccc3c2CC(F)(F)F)cc1F ; CC(=O)NS(=O)(=O)c1cc(OC(F)F)c(NCC#Cc2sc3c(NC4CCC(N5CC6(CCOCC6)C5)CC4)cccc3c2CC(F)(F)F)cc1F] 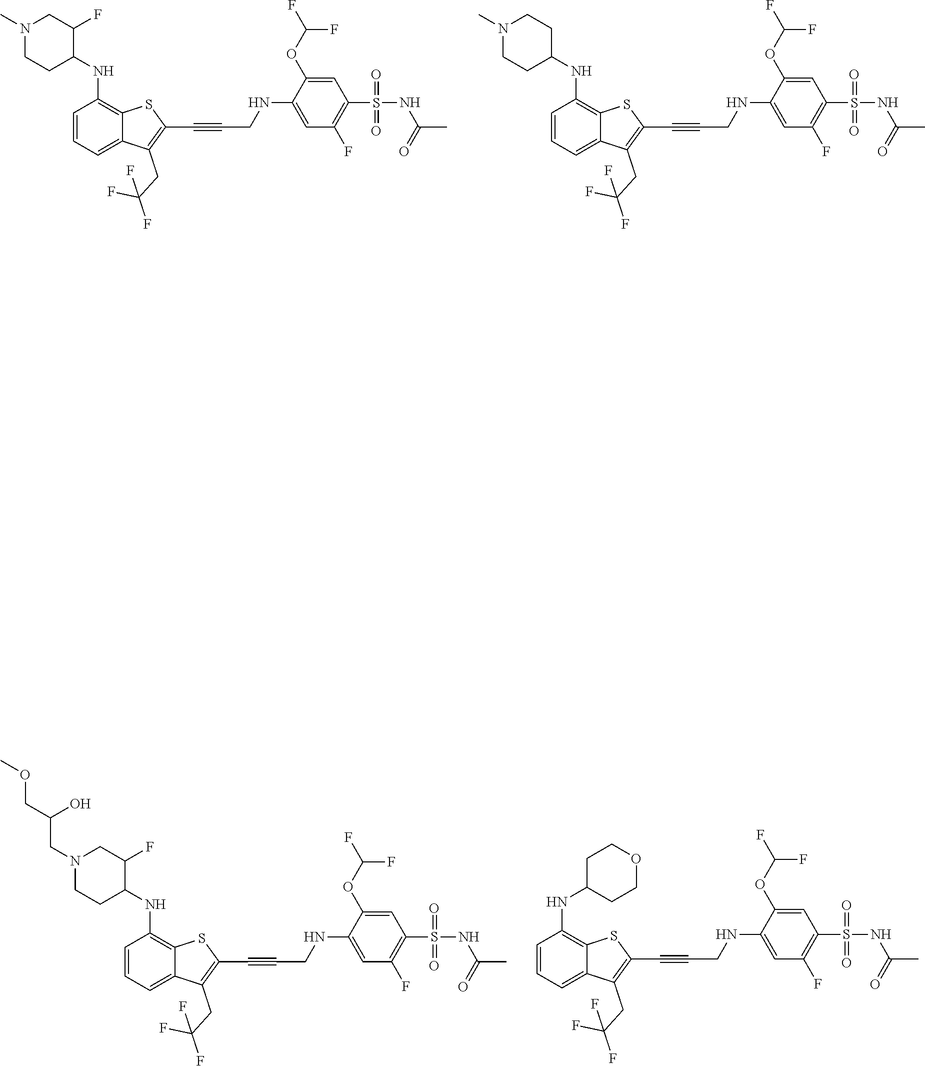 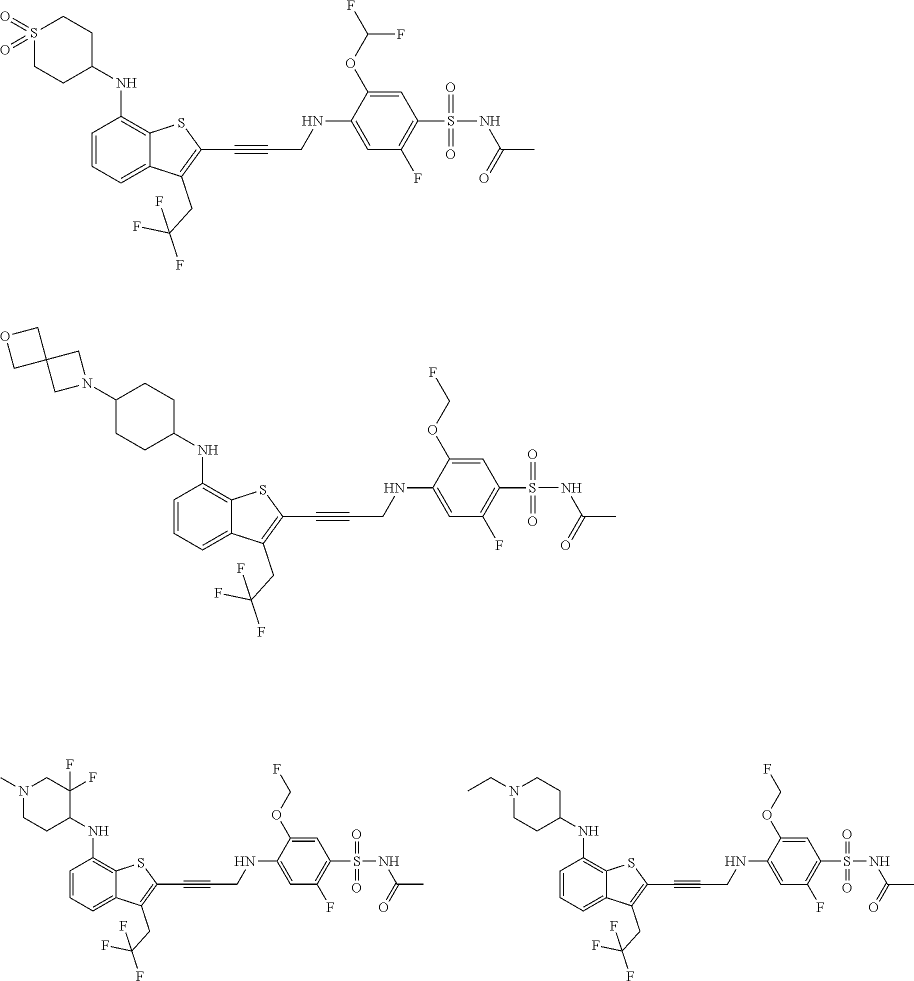 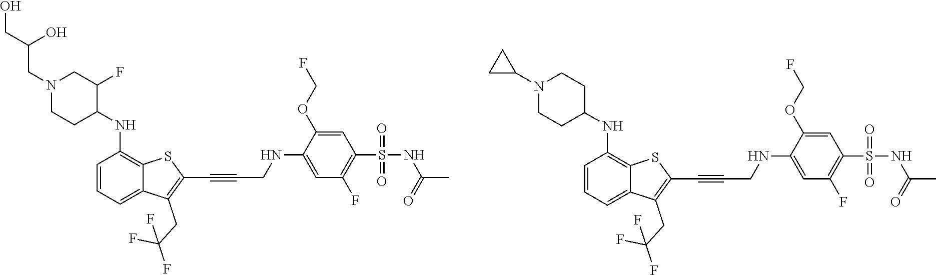 [SMILES: CC(=O)NS(=O)(=O)c1cc(OCF)c(NCC#Cc2sc3c(NC4CCN(CC(O)CO)CC4F)cccc3c2CC(F)(F)F)cc1F ; CC(=O)NS(=O)(=O)c1cc(OCF)c(NCC#Cc2sc3c(NC4CCN(C5CC5)CC4)cccc3c2CC(F)(F)F)cc1F] 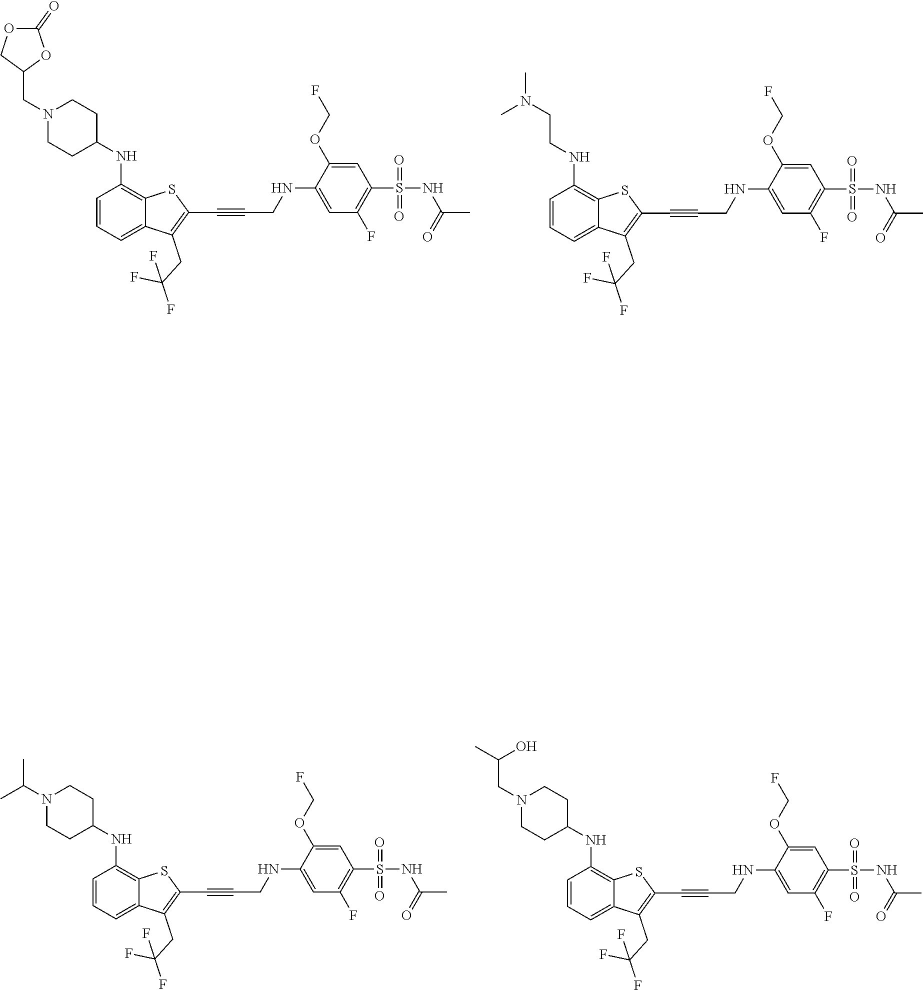 [SMILES: CC(=O)NS(=O)(=O)c1cc(OCF)c(NCC#Cc2sc3c(NC4CCN(CC5COC(=O)O5)CC4)cccc3c2CC(F)(F)F)cc1F ; CC(=O)NS(=O)(=O)c1cc(OCF)c(NCC#Cc2sc3c(NCCN(C)C)cccc3c2CC(F)(F)F)cc1F ; CC(=O)NS(=O)(=O)c1cc(OCF)c(NCC#Cc2sc3c(NC4CCN(C(C)C)CC4)cccc3c2CC(F)(F)F)cc1F ; CC(=O)NS(=O)(=O)c1cc(OCF)c(NCC#Cc2sc3c(NC4CCN(CC(C)O)CC4)cccc3c2CC(F)(F)F)cc1F] 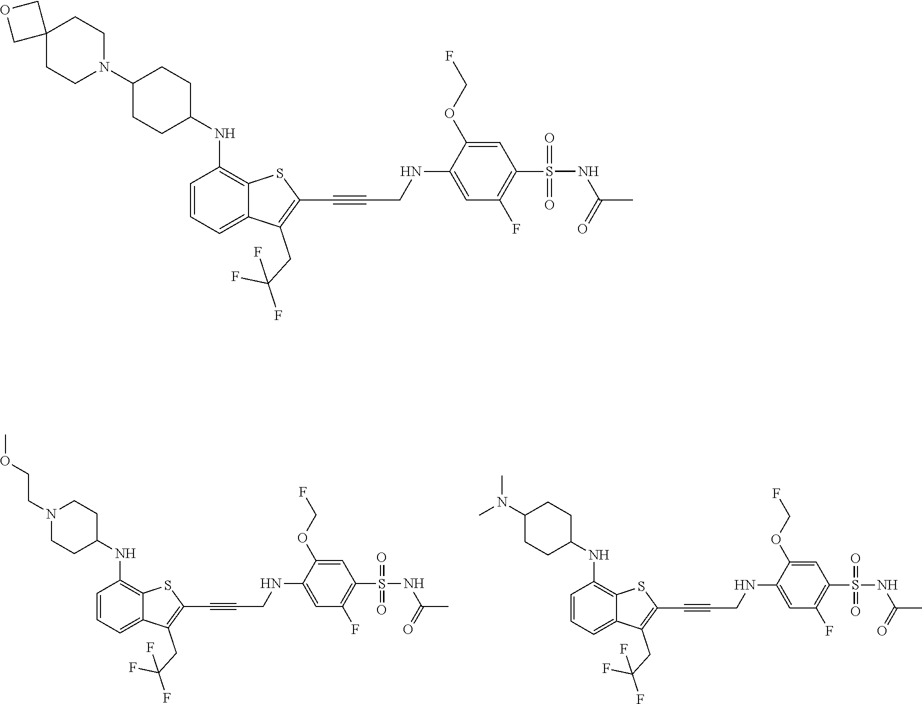 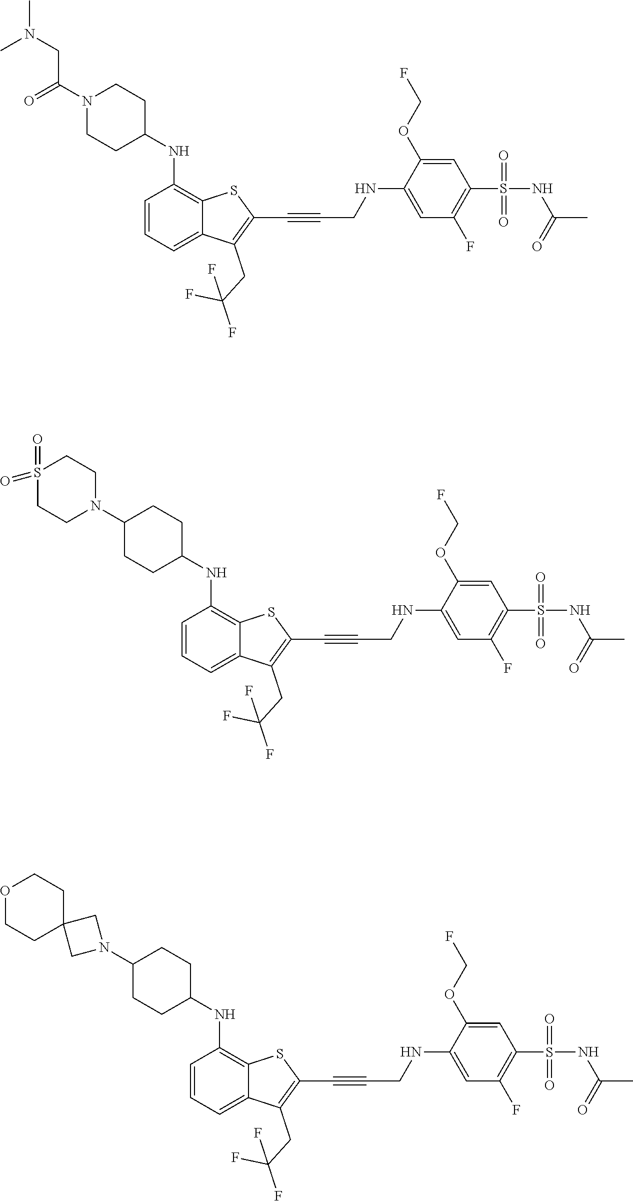 [SMILES: CC(=O)NS(=O)(=O)c1cc(OCF)c(NCC#Cc2sc3c(NC4CCN(C(=O)CN(C)C)CC4)cccc3c2CC(F)(F)F)cc1F ; CC(=O)NS(=O)(=O)c1cc(OCF)c(NCC#Cc2sc3c(NC4CCC(N5CCS(=O)(=O)CC5)CC4)cccc3c2CC(F)(F)F)cc1F ; CC(=O)NS(=O)(=O)c1cc(OCF)c(NCC#Cc2sc3c(NC4CCC(N5CC6(CCOCC6)C5)CC4)cccc3c2CC(F)(F)F)cc1F] 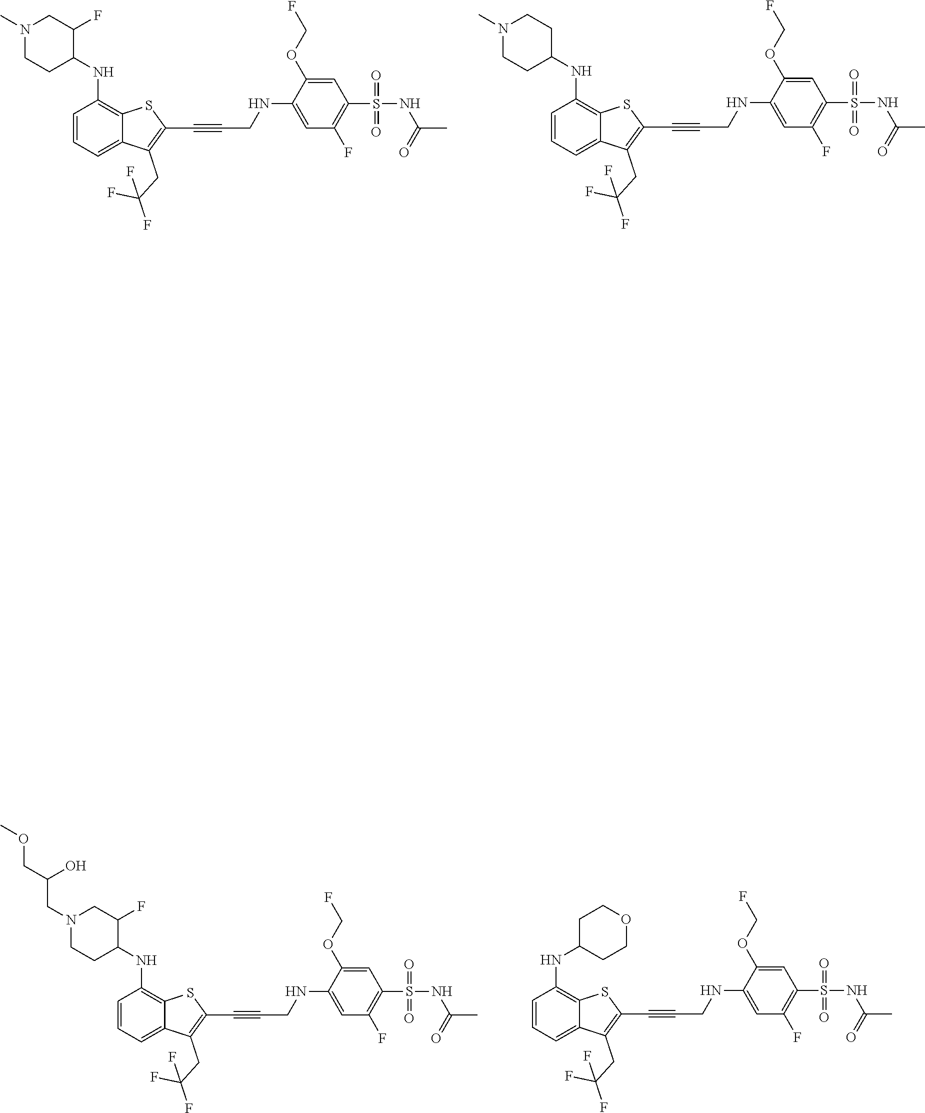 [SMILES: CC(=O)NS(=O)(=O)c1cc(OCF)c(NCC#Cc2sc3c(NC4CCN(C)CC4F)cccc3c2CC(F)(F)F)cc1F ; CC(=O)NS(=O)(=O)c1cc(OCF)c(NCC#Cc2sc3c(NC4CCN(C)CC4)cccc3c2CC(F)(F)F)cc1F ; COCC(O)CN1CCC(Nc2cccc3c(CC(F)(F)F)c(C#CCNc4cc(F)c(S(=O)(=O)NC(C)=O)cc4OCF)sc23)C(F)C1 ; CC(=O)NS(=O)(=O)c1cc(OCF)c(NCC#Cc2sc3c(NC4CCOCC4)cccc3c2CC(F)(F)F)cc1F] 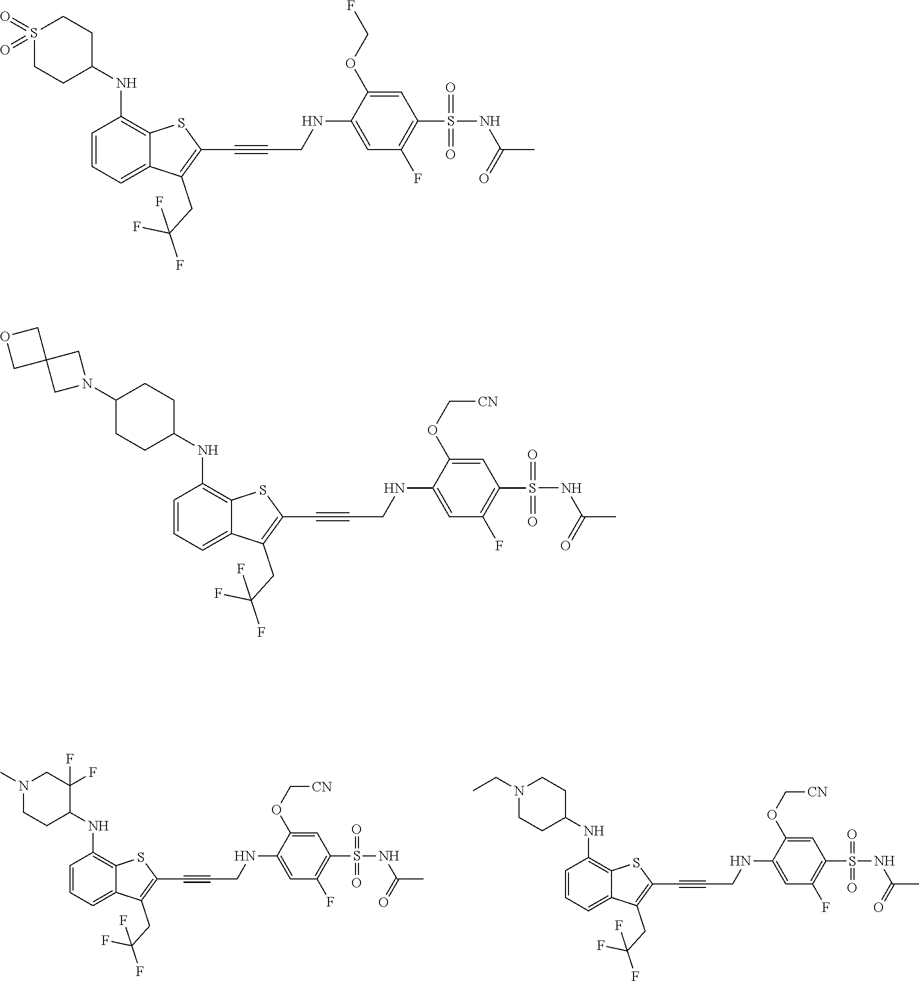 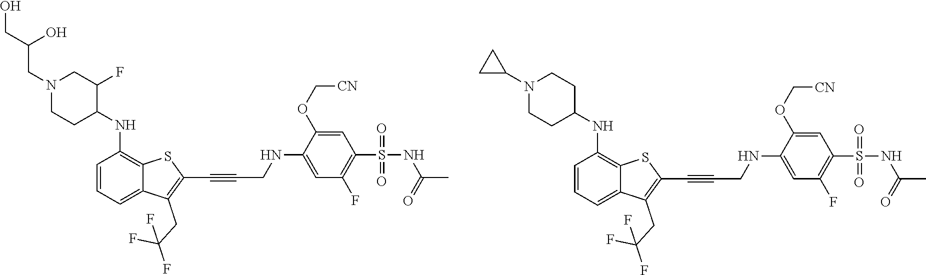 [SMILES: CC(=O)NS(=O)(=O)c1cc(OCC#N)c(NCC#Cc2sc3c(NC4CCN(CC(O)CO)CC4F)cccc3c2CC(F)(F)F)cc1F ; CC(=O)NS(=O)(=O)c1cc(OCC#N)c(NCC#Cc2sc3c(NC4CCN(C5CC5)CC4)cccc3c2CC(F)(F)F)cc1F] 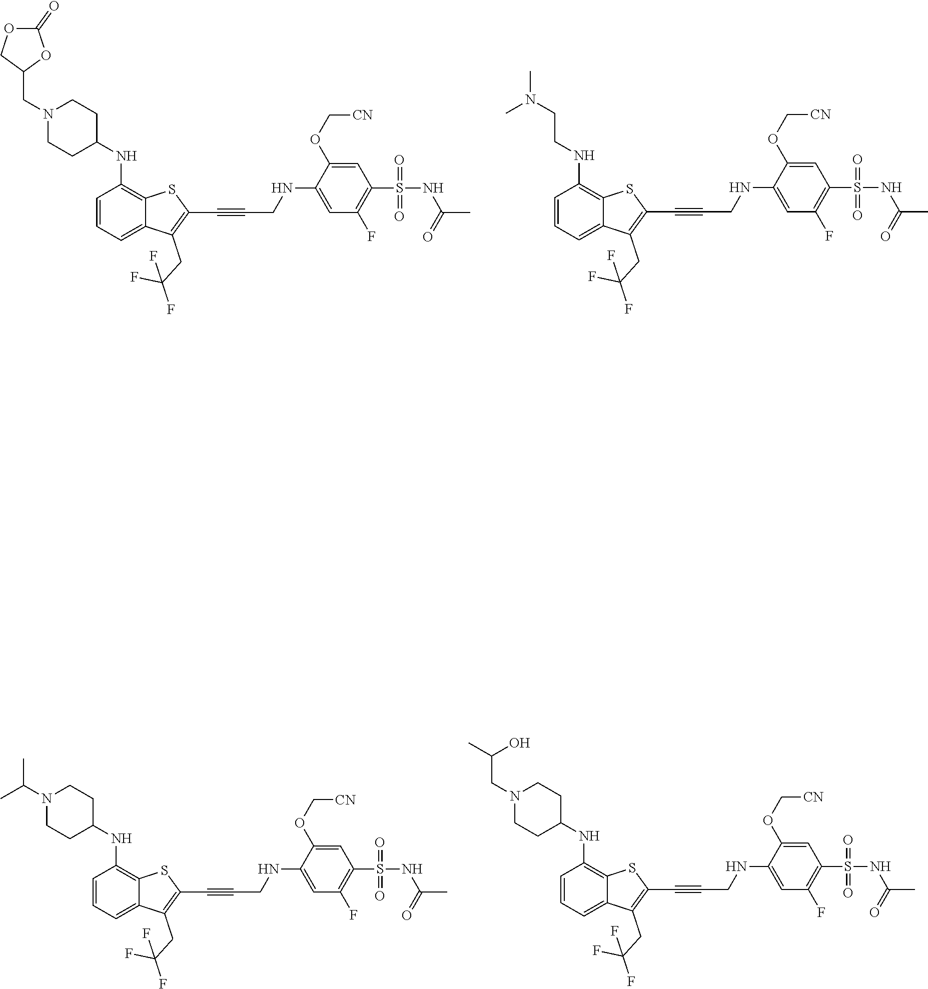 [SMILES: CC(=O)NS(=O)(=O)c1cc(OCC#N)c(NCC#Cc2sc3c(NC4CCN(CC5COC(=O)O5)CC4)cccc3c2CC(F)(F)F)cc1F ; CC(=O)NS(=O)(=O)c1cc(OCC#N)c(NCC#Cc2sc3c(NCCN(C)C)cccc3c2CC(F)(F)F)cc1F ; CC(=O)NS(=O)(=O)c1cc(OCC#N)c(NCC#Cc2sc3c(NC4CCN(C(C)C)CC4)cccc3c2CC(F)(F)F)cc1F ; CC(=O)NS(=O)(=O)c1cc(OCC#N)c(NCC#Cc2sc3c(NC4CCN(CC(C)O)CC4)cccc3c2CC(F)(F)F)cc1F] 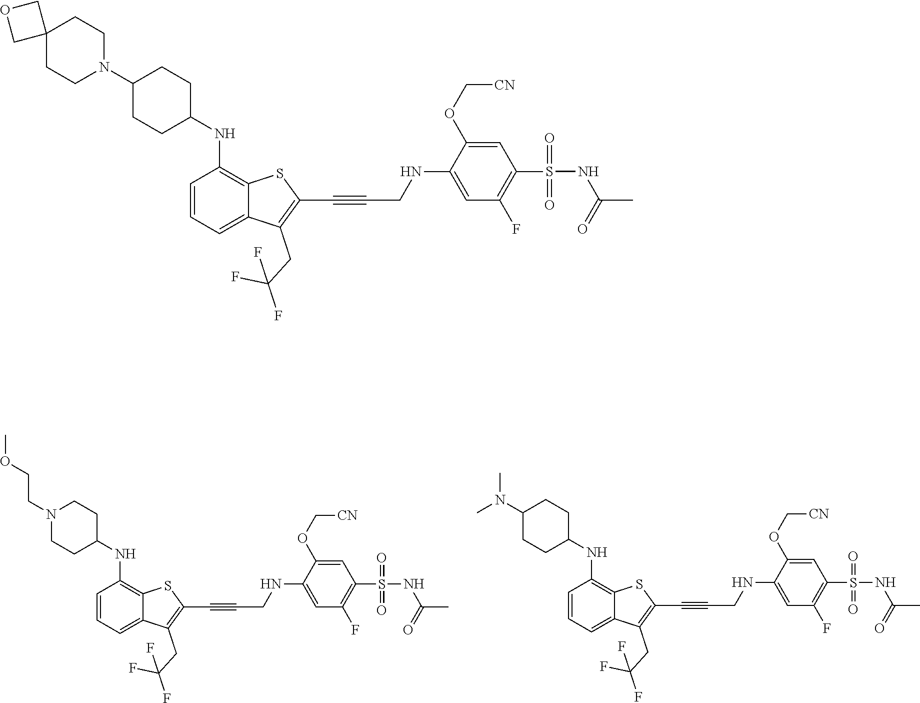 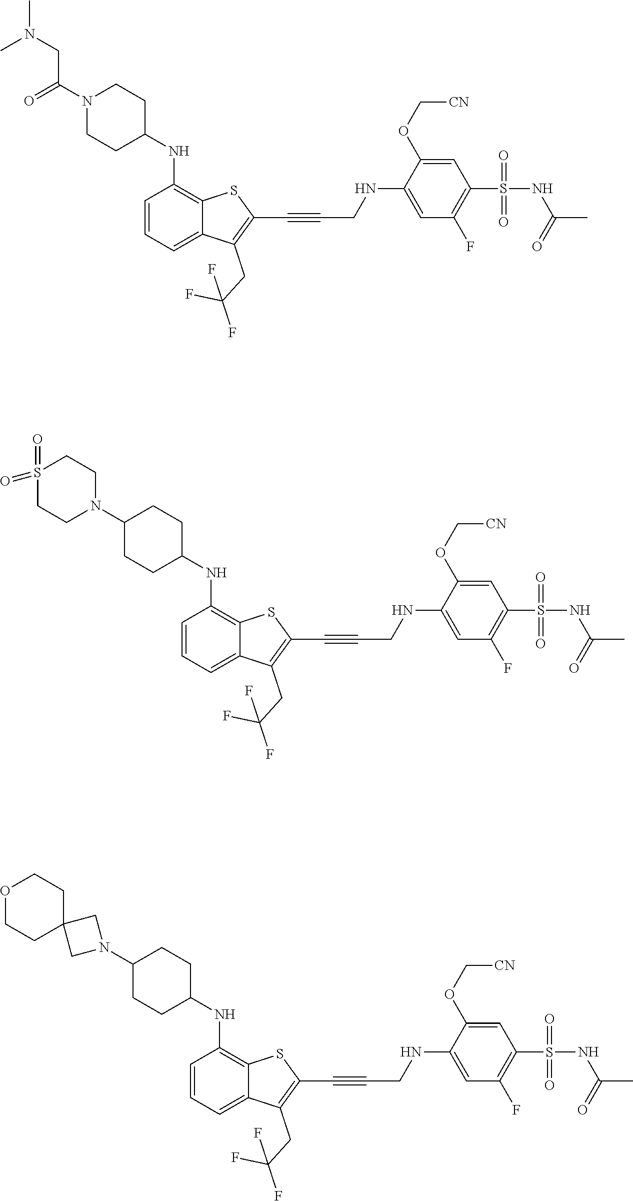 [SMILES: CC(=O)NS(=O)(=O)c1cc(OCC#N)c(NCC#Cc2sc3c(NC4CCN(C(=O)CN(C)C)CC4)cccc3c2CC(F)(F)F)cc1F ; CC(=O)NS(=O)(=O)c1cc(OCC#N)c(NCC#Cc2sc3c(NC4CCC(N5CCS(=O)(=O)CC5)CC4)cccc3c2CC(F)(F)F)cc1F ; CC(=O)NS(=O)(=O)c1cc(OCC#N)c(NCC#Cc2sc3c(NC4CCC(N5CC6(CCOCC6)C5)CC4)cccc3c2CC(F)(F)F)cc1F] 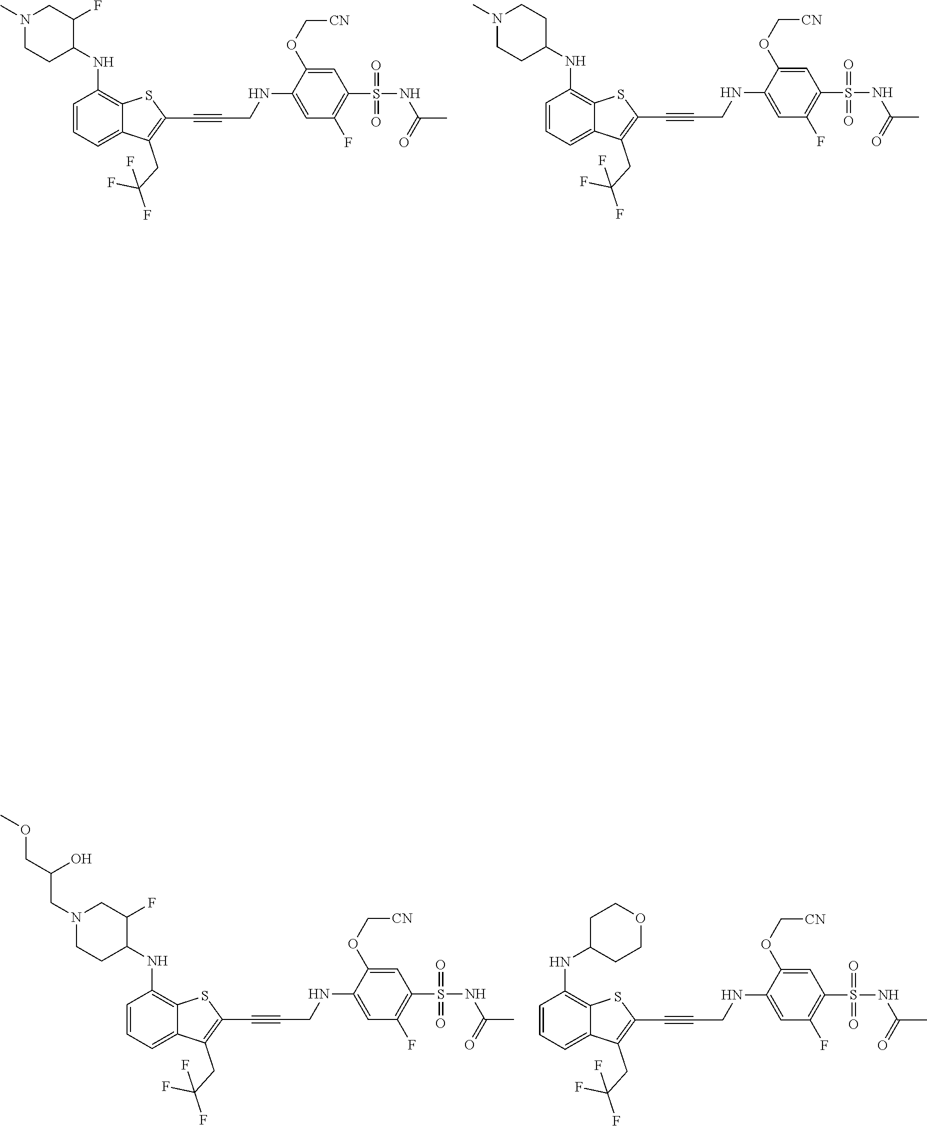 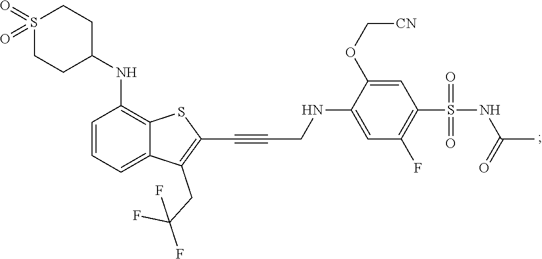 [SMILES: CC(=O)NS(=O)(=O)c1cc(OCC#N)c(NCC#Cc2sc3c(NC4CCS(=O)(=O)CC4)cccc3c2CC(F)(F)F)cc1F]
or the compound of formula (I) is selected from: 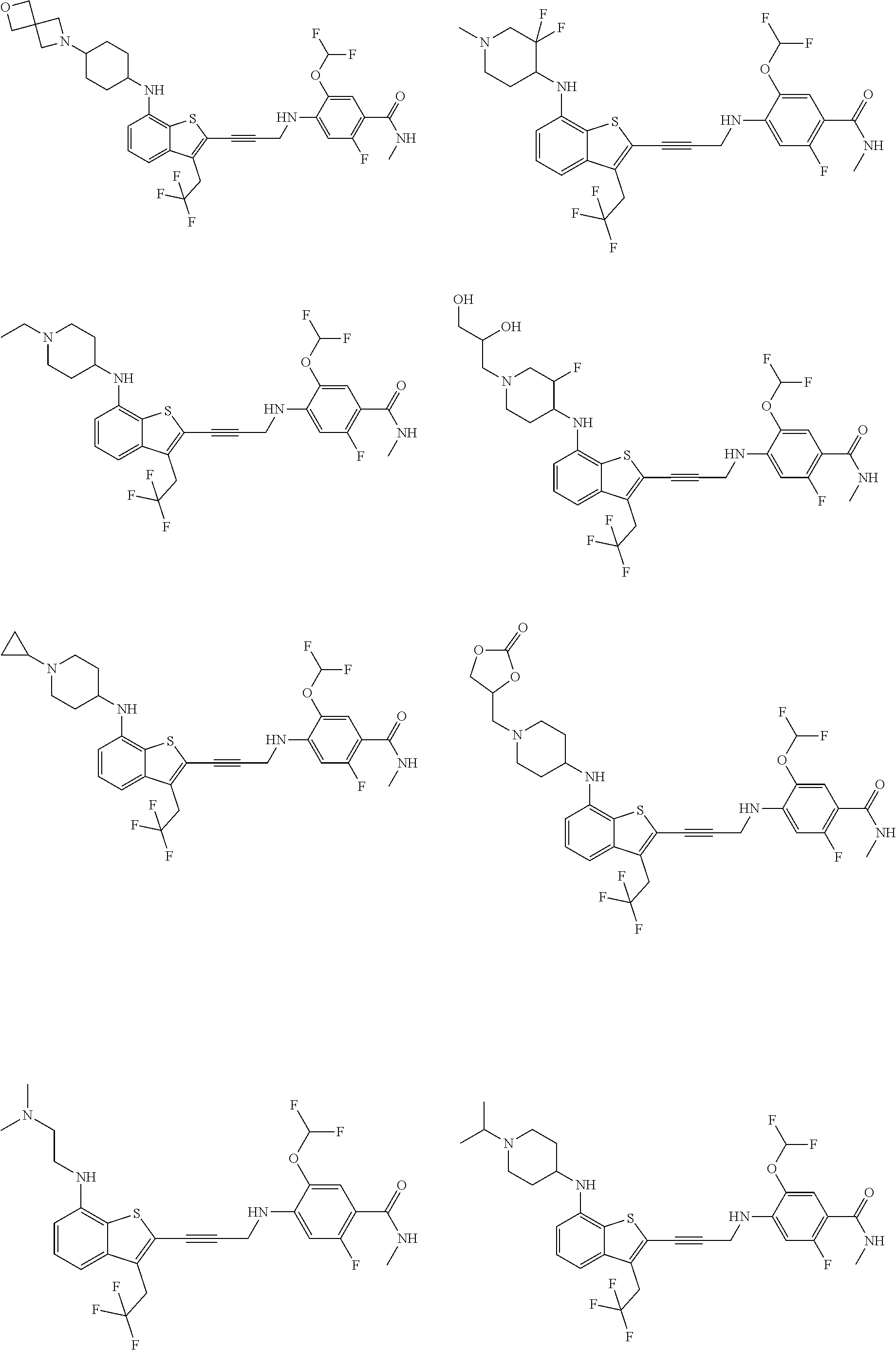 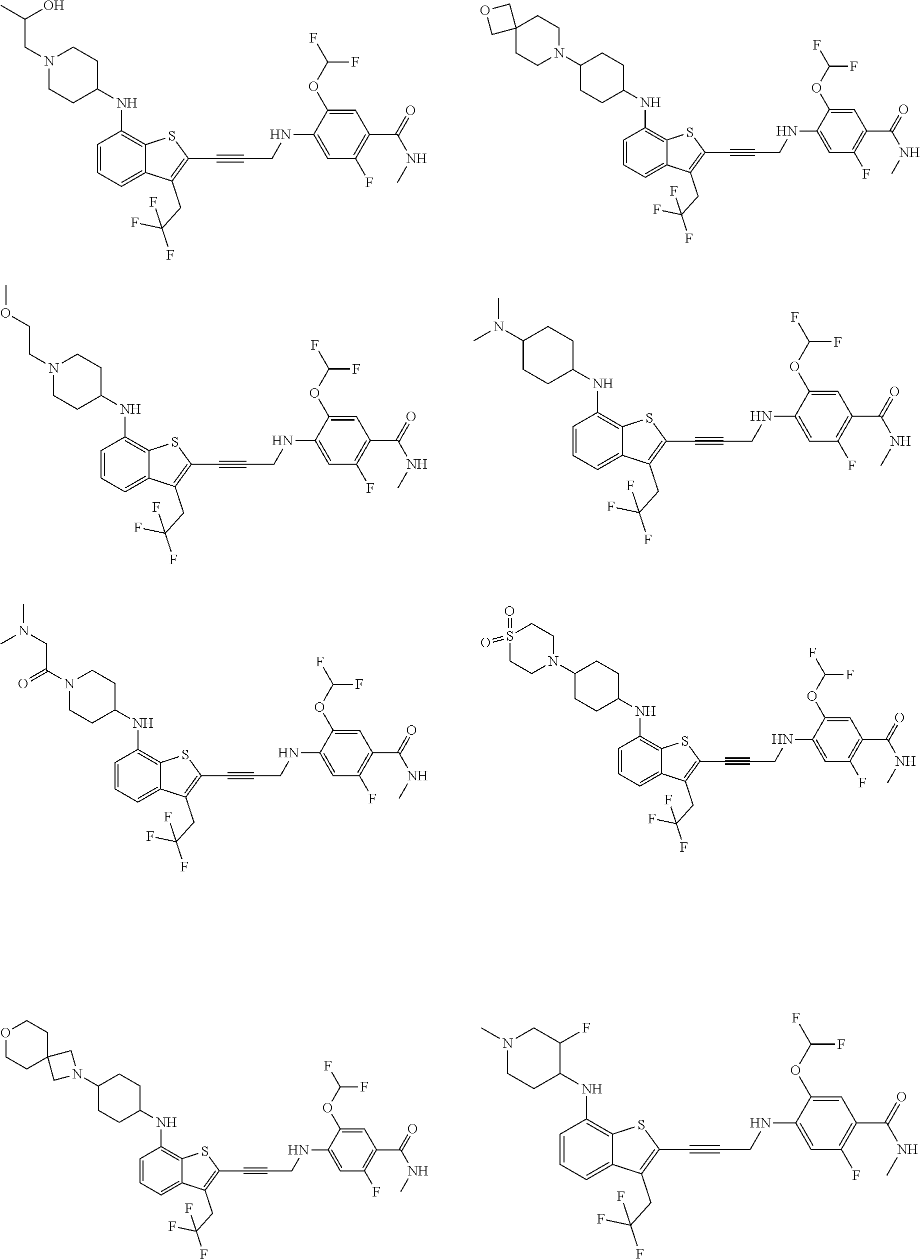 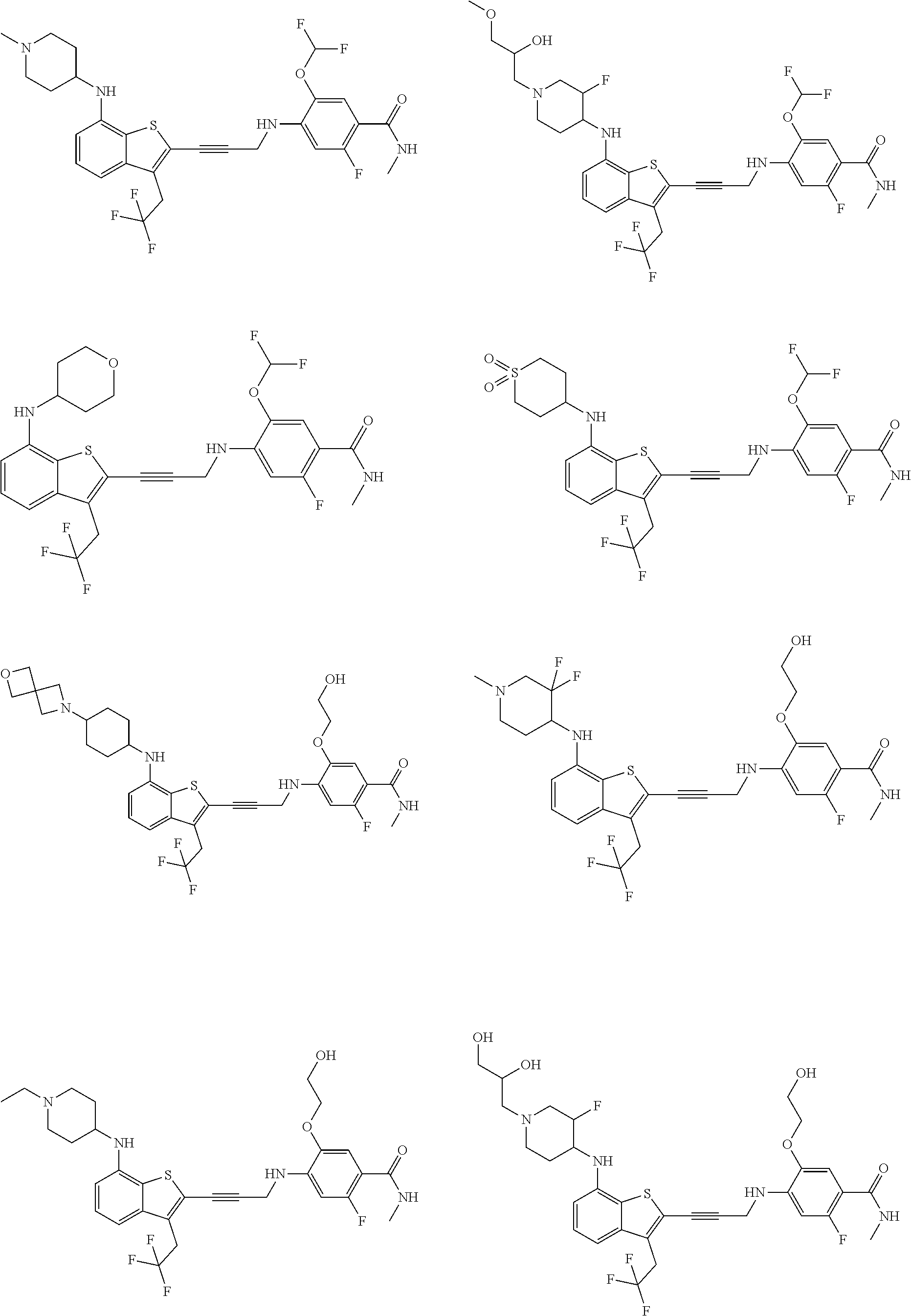 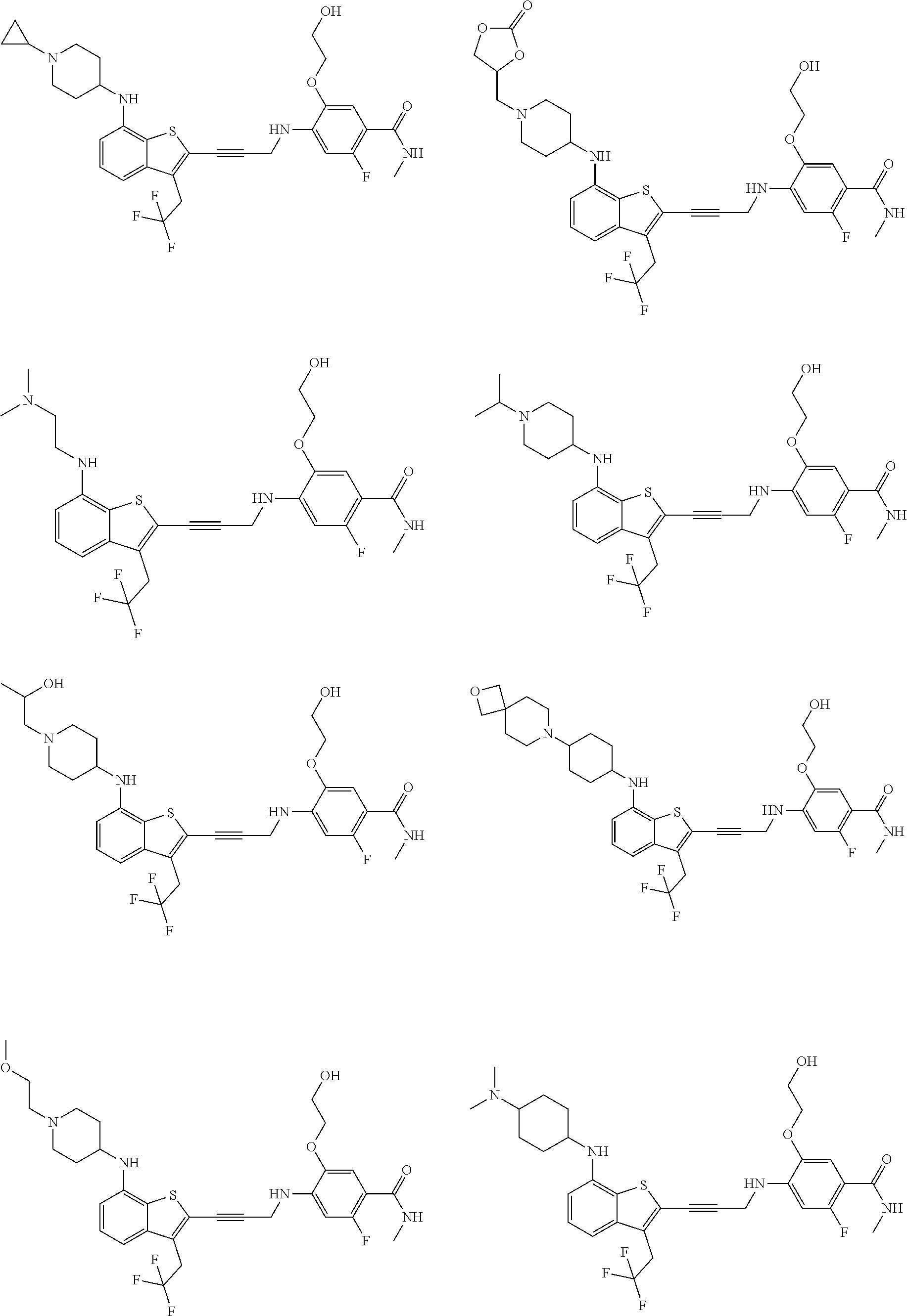 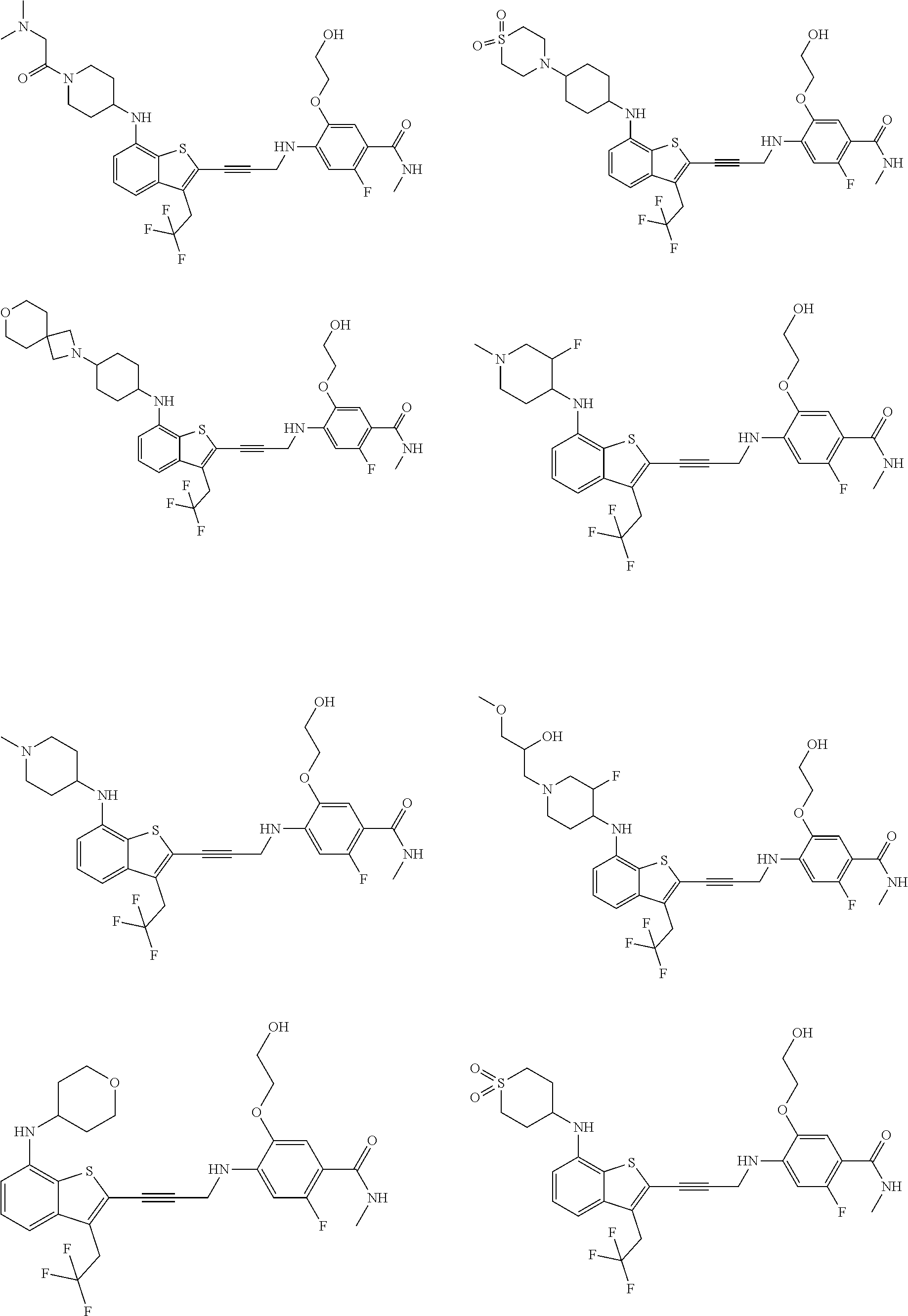 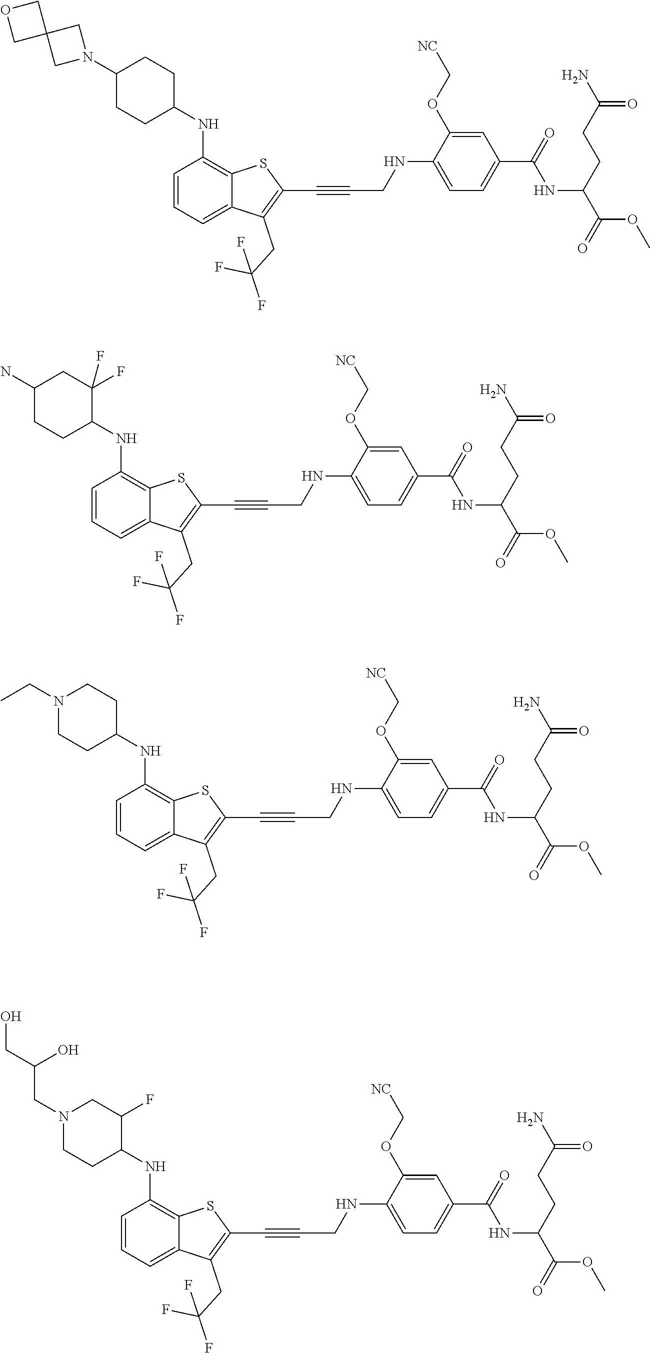 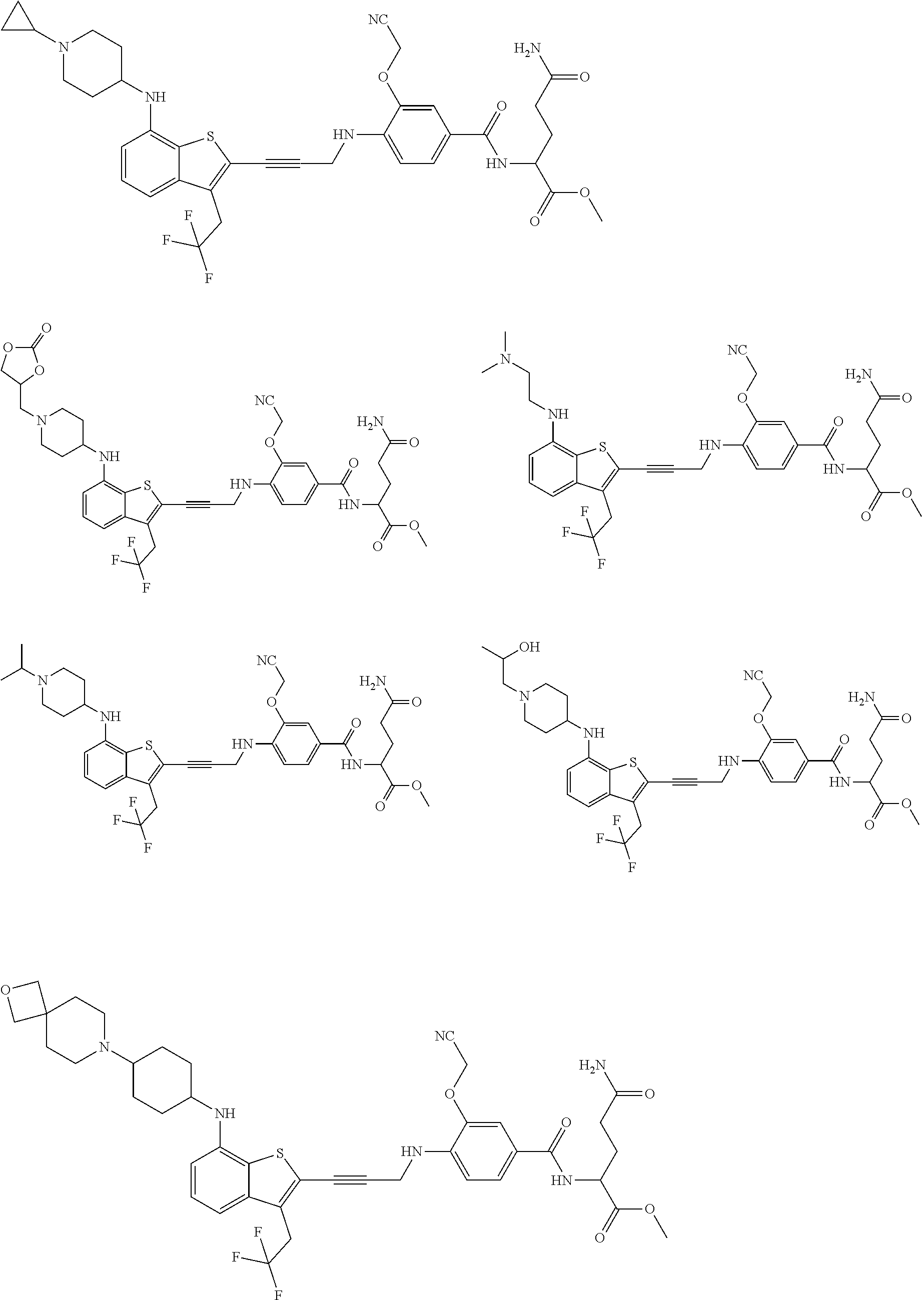 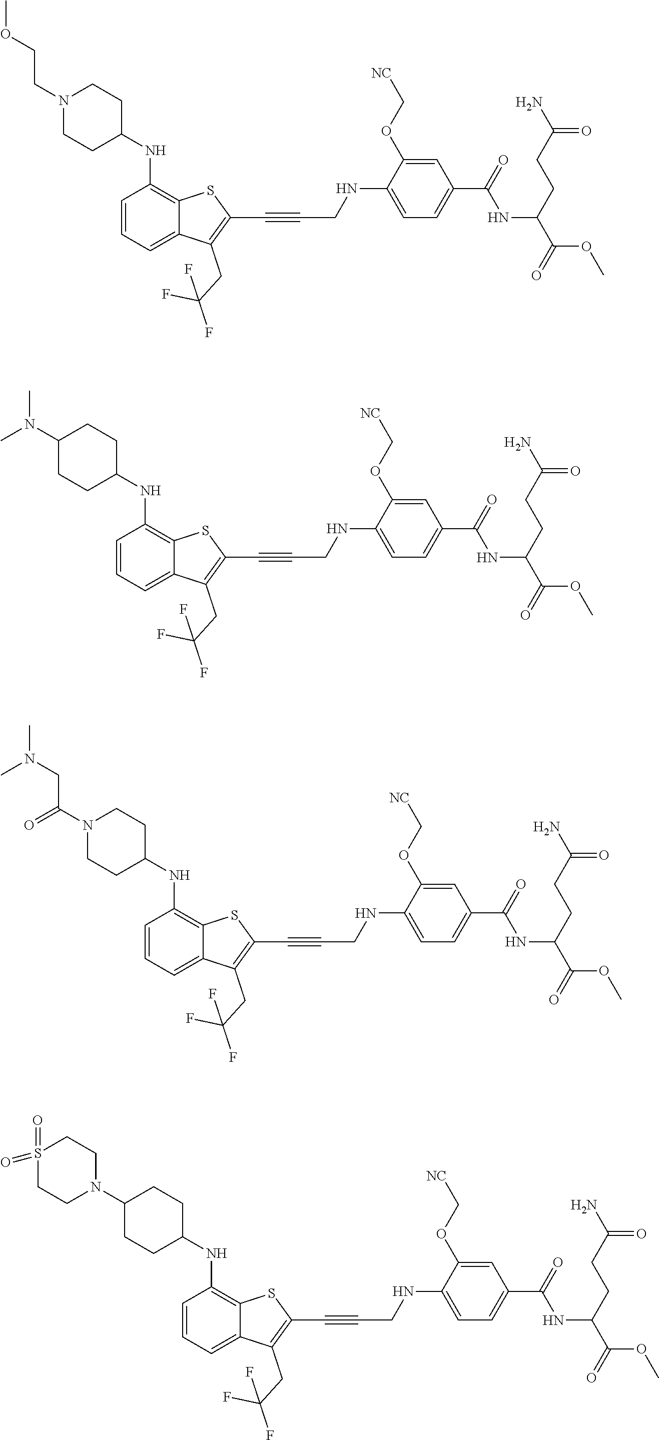 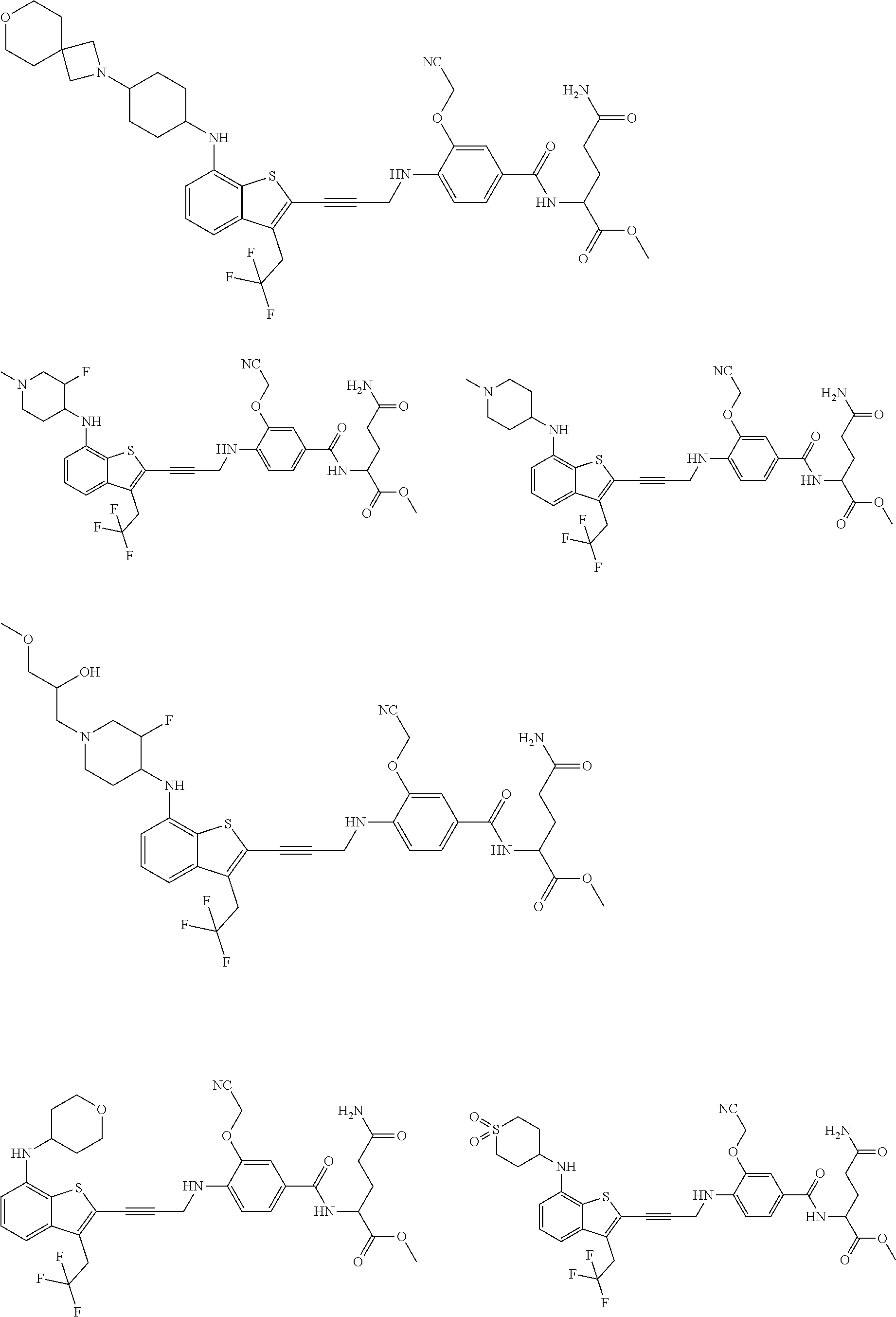 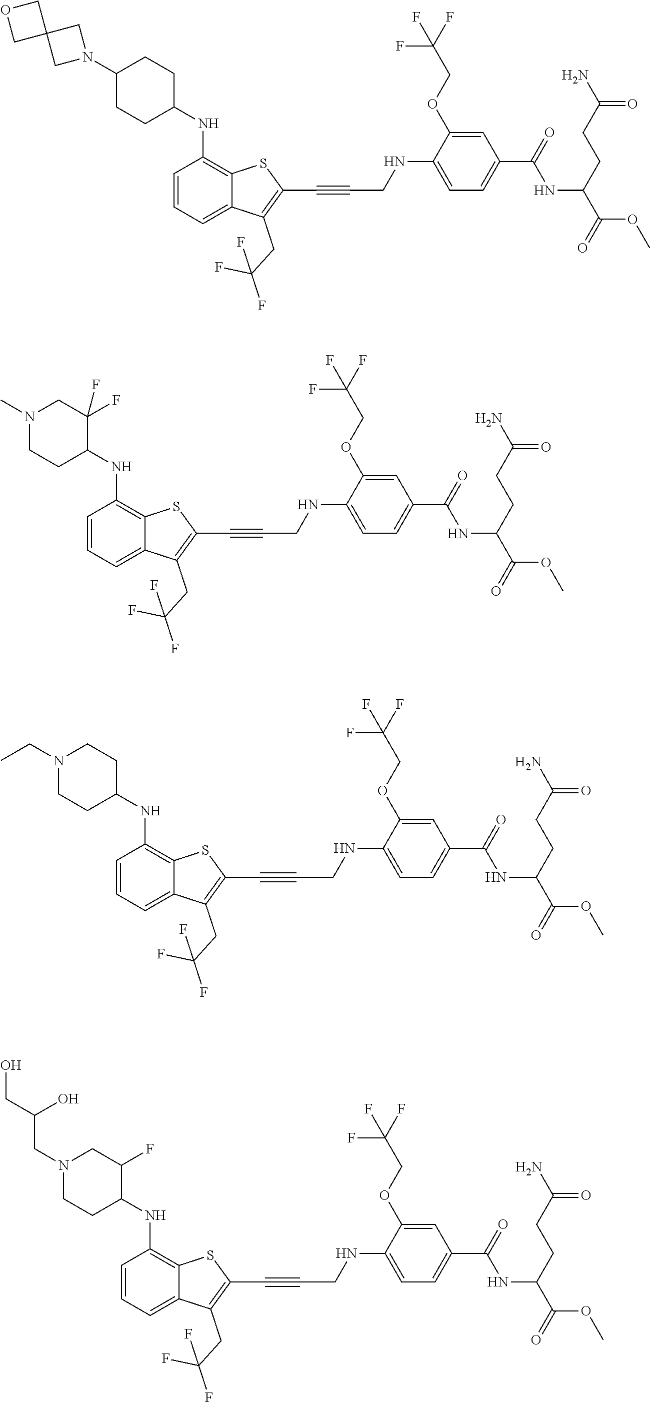 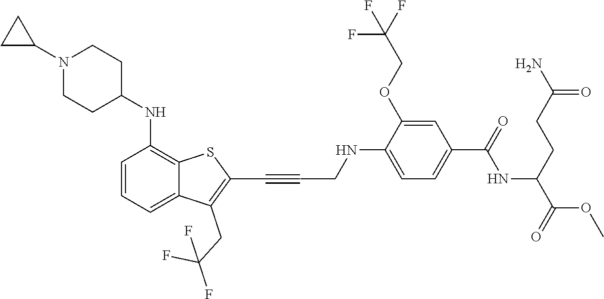 [SMILES: COC(=O)C(CCC(N)=O)NC(=O)c1ccc(NCC#Cc2sc3c(NC4CCN(C5CC5)CC4)cccc3c2CC(F)(F)F)c(OCC(F)(F)F)c1] 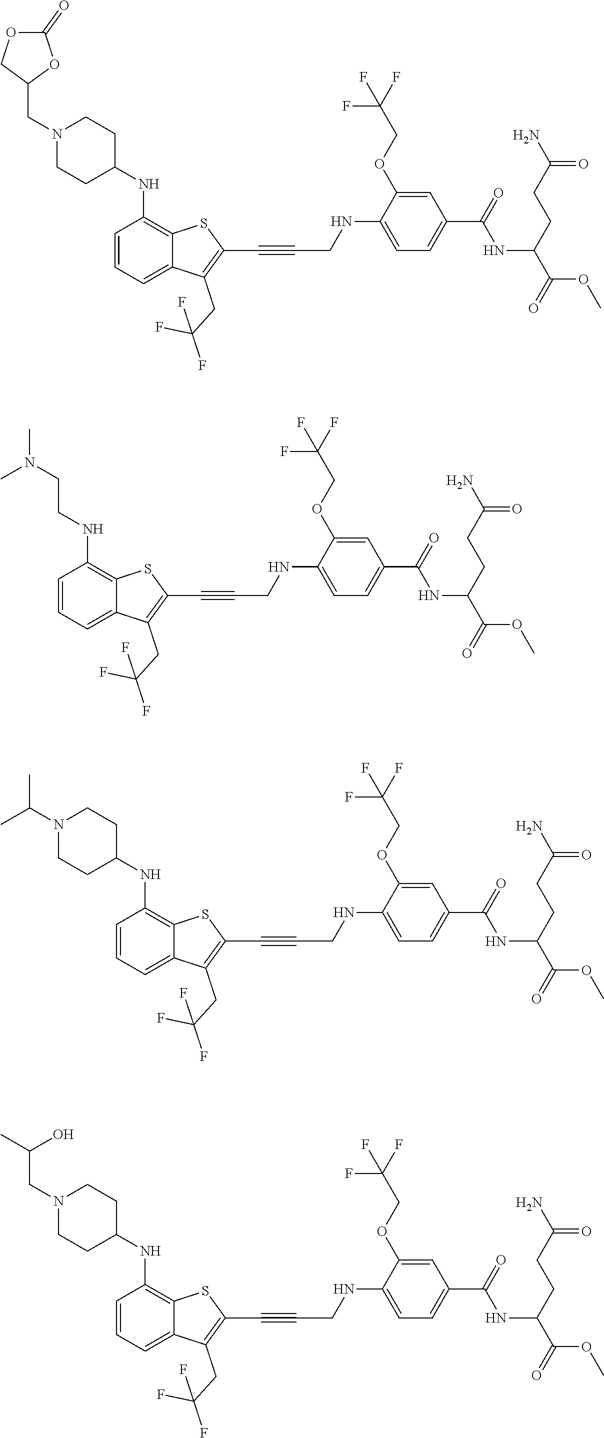 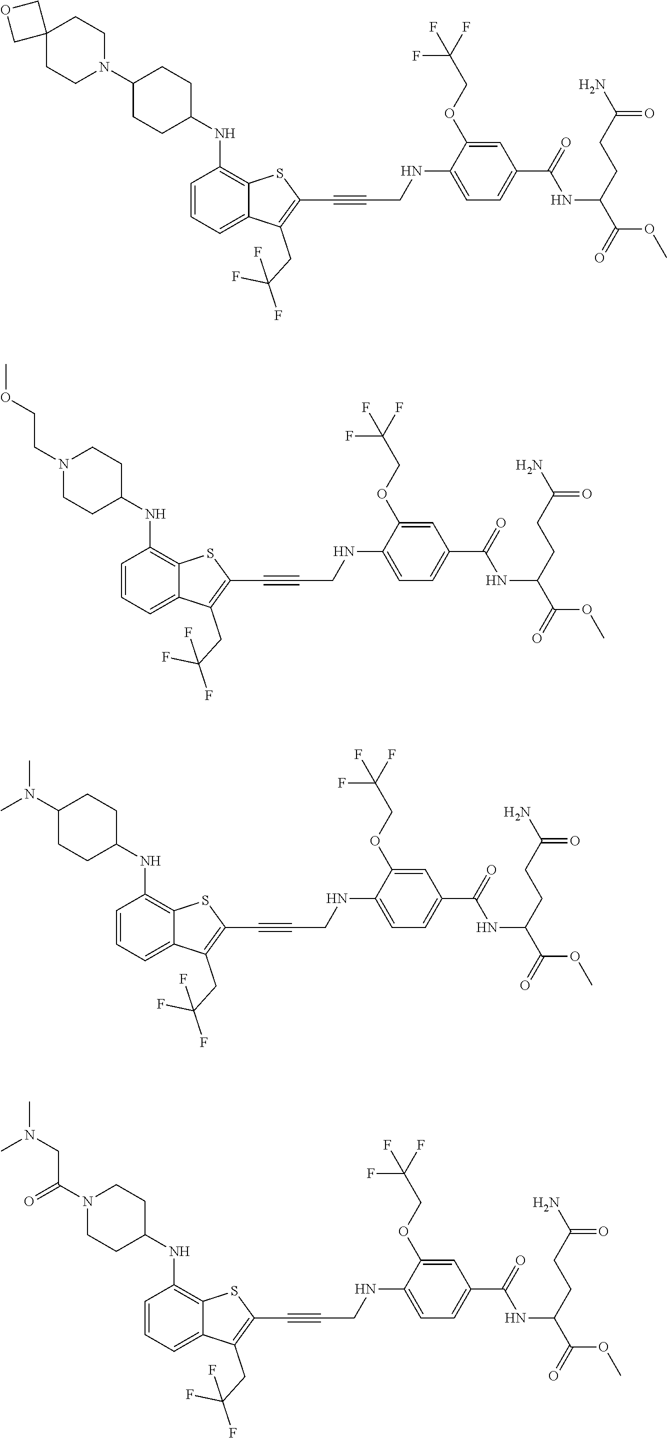 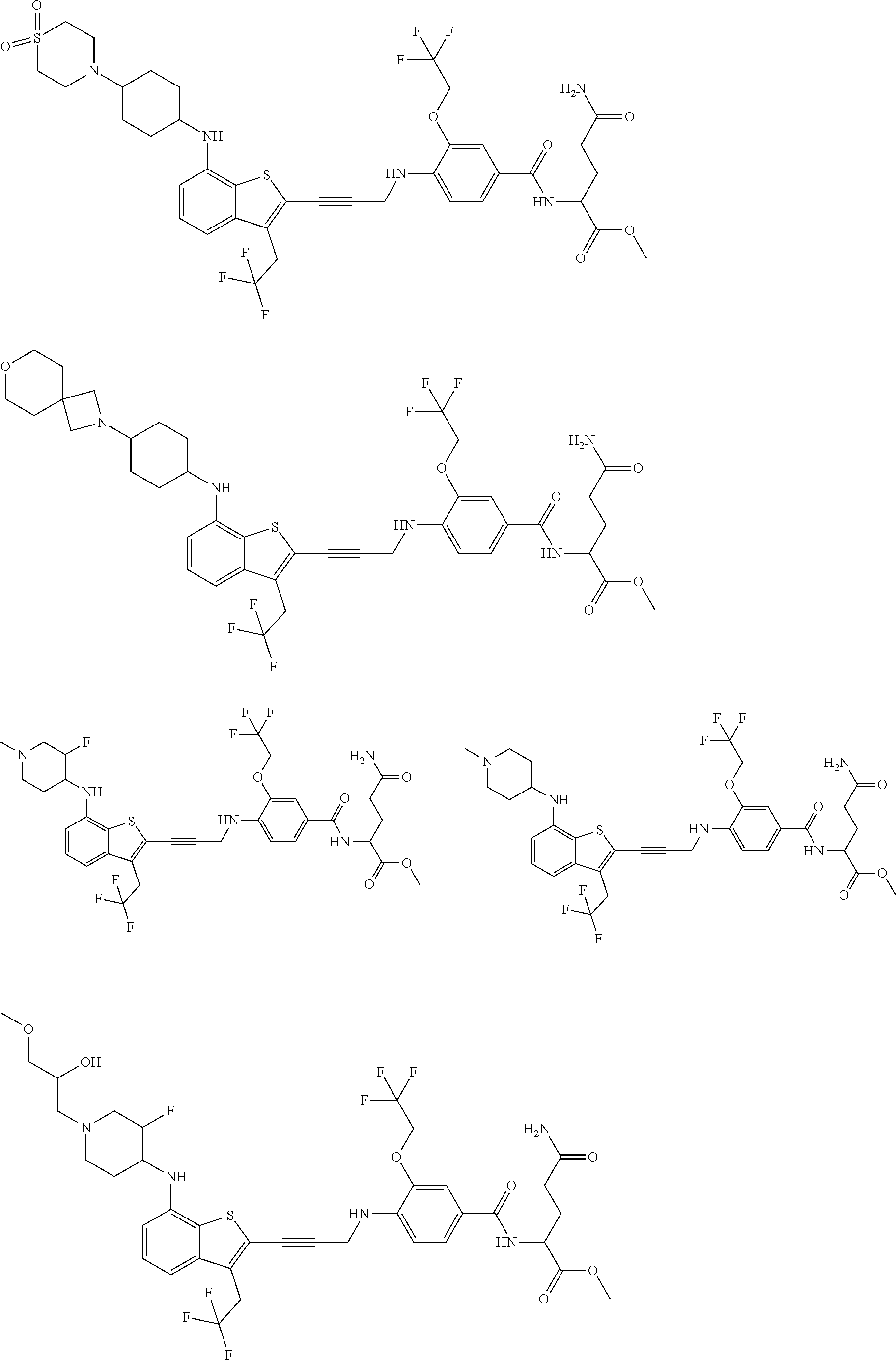 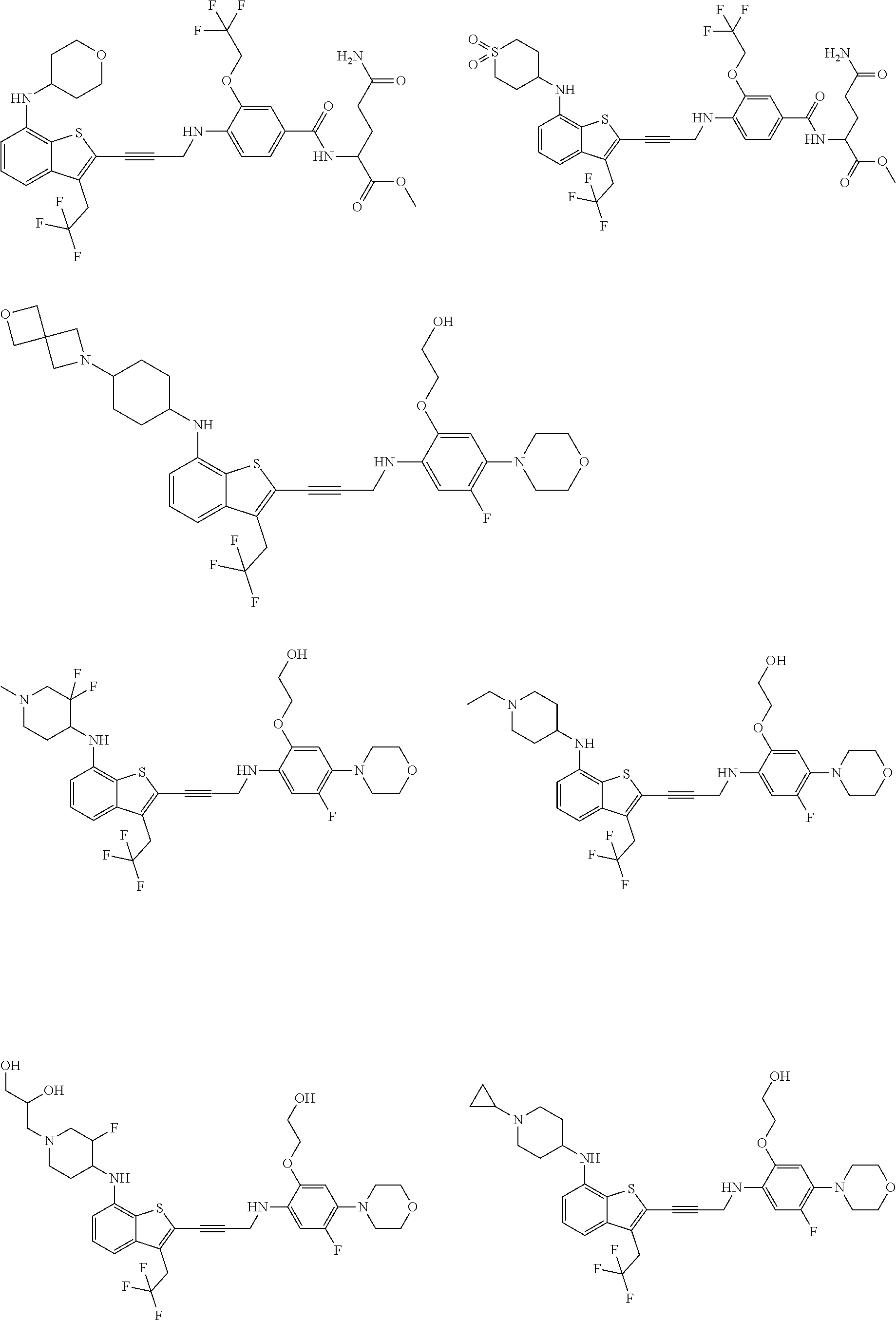 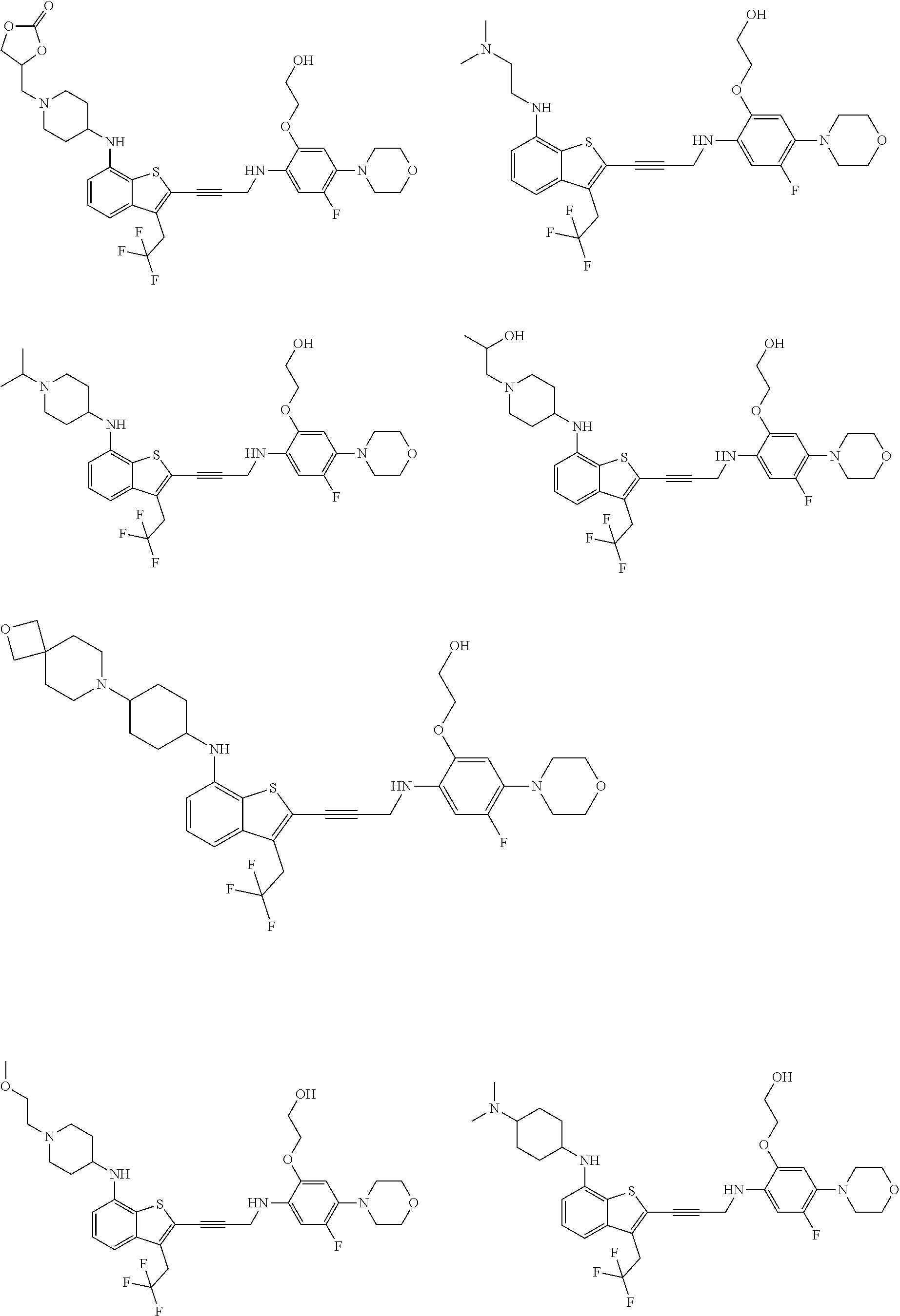 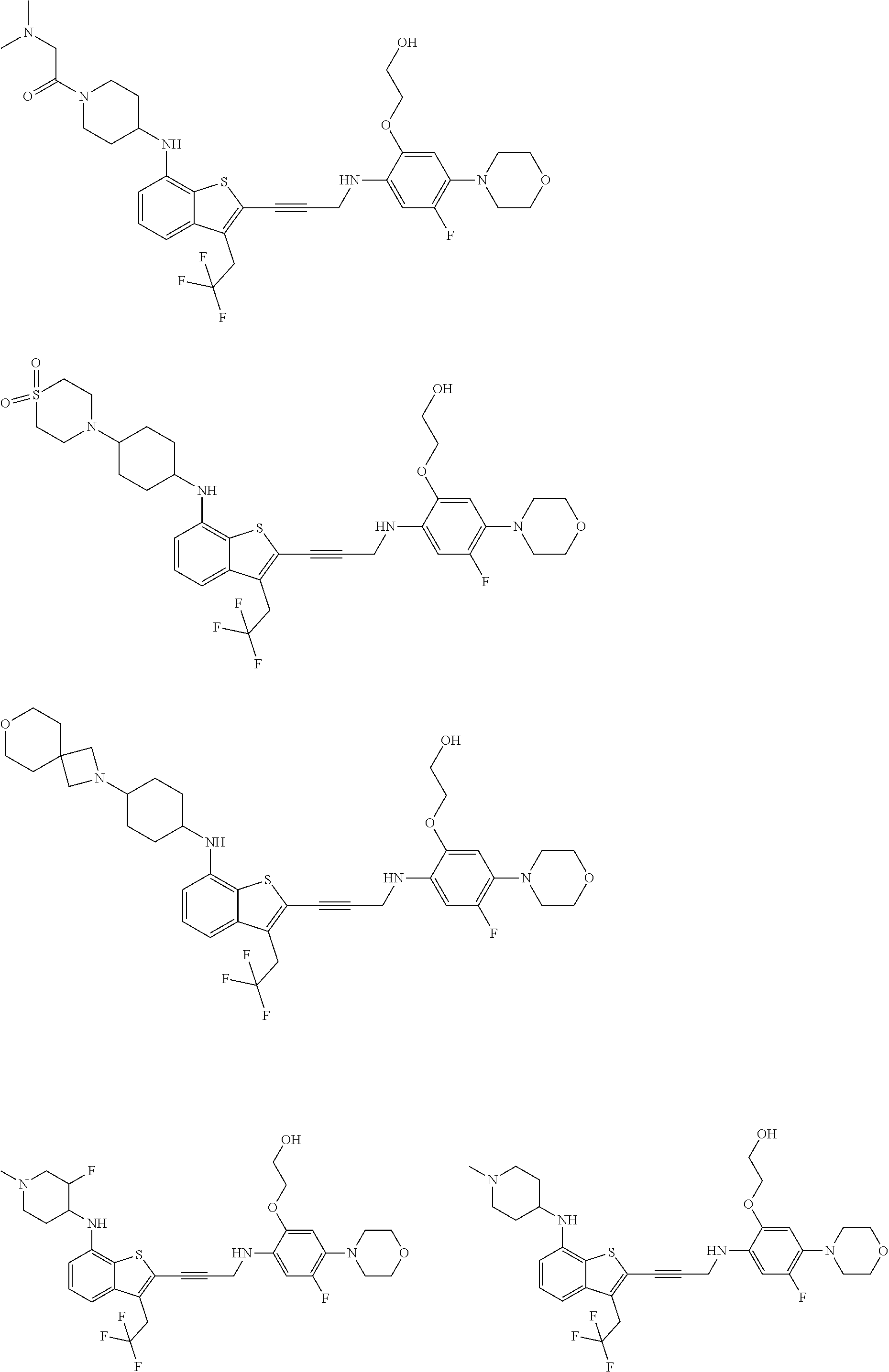 [SMILES: CN(C)CC(=O)N1CCC(Nc2cccc3c(CC(F)(F)F)c(C#CCNc4cc(F)c(N5CCOCC5)cc4OCCO)sc23)CC1 ; O=S1(=O)CCN(C2CCC(Nc3cccc4c(CC(F)(F)F)c(C#CCNc5cc(F)c(N6CCOCC6)cc5OCCO)sc34)CC2)CC1 ; OCCOc1cc(N2CCOCC2)c(F)cc1NCC#Cc1sc2c(NC3CCC(N4CC5(CCOCC5)C4)CC3)cccc2c1CC(F)(F)F ; CN1CCC(Nc2cccc3c(CC(F)(F)F)c(C#CCNc4cc(F)c(N5CCOCC5)cc4OCCO)sc23)C(F)C1 ; CN1CCC(Nc2cccc3c(CC(F)(F)F)c(C#CCNc4cc(F)c(N5CCOCC5)cc4OCCO)sc23)CC1] 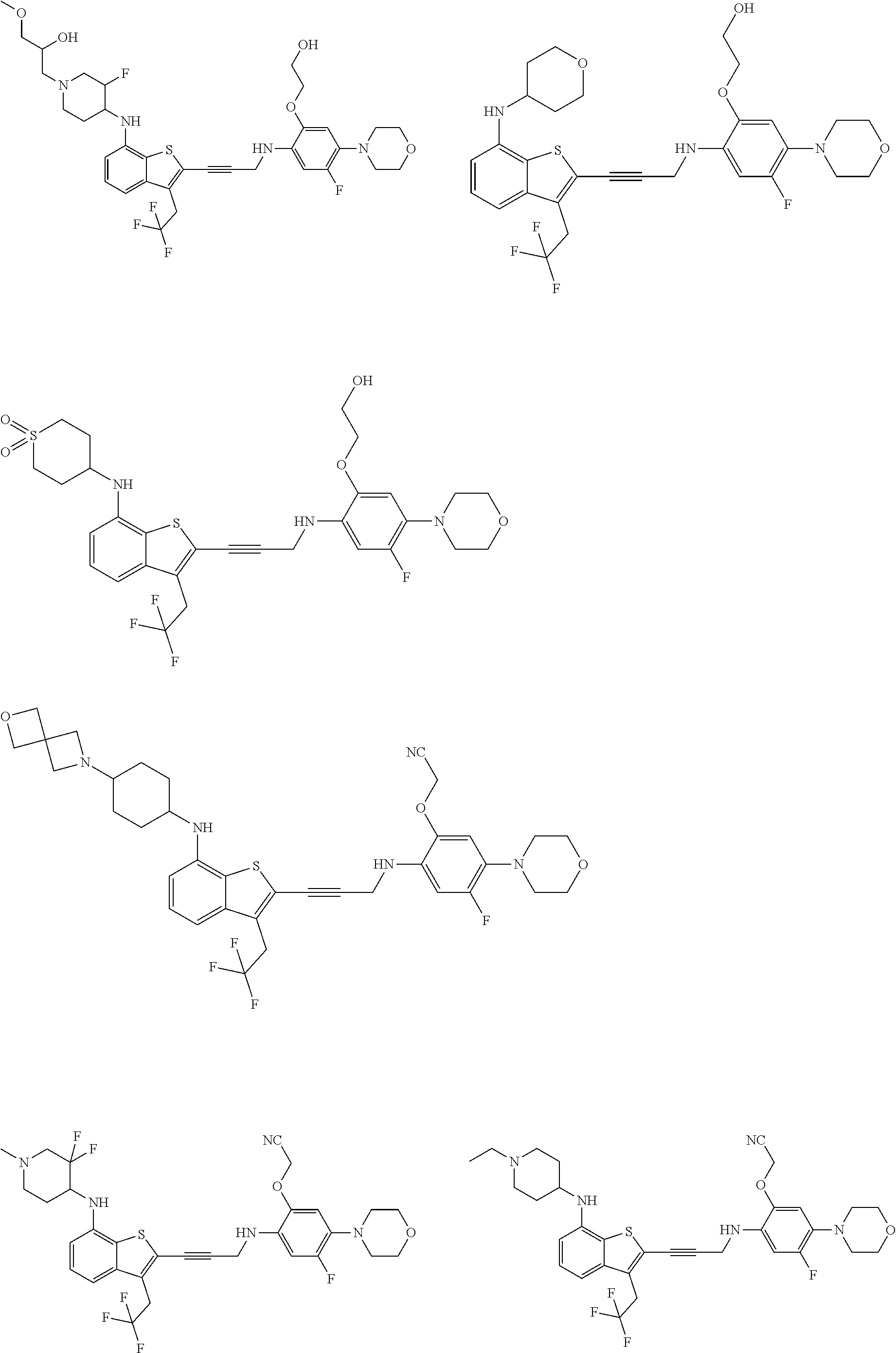 [SMILES: COCC(O)CN1CCC(Nc2cccc3c(CC(F)(F)F)c(C#CCNc4cc(F)c(N5CCOCC5)cc4OCCO)sc23)C(F)C1 ; OCCOc1cc(N2CCOCC2)c(F)cc1NCC#Cc1sc2c(NC3CCOCC3)cccc2c1CC(F)(F)F ; O=S1(=O)CCC(Nc2cccc3c(CC(F)(F)F)c(C#CCNc4cc(F)c(N5CCOCC5)cc4OCCO)sc23)CC1 ; N#CCOc1cc(N2CCOCC2)c(F)cc1NCC#Cc1sc2c(NC3CCC(N4CC5(COC5)C4)CC3)cccc2c1CC(F)(F)F ; CN1CCC(Nc2cccc3c(CC(F)(F)F)c(C#CCNc4cc(F)c(N5CCOCC5)cc4OCC#N)sc23)C(F)(F)C1 ; CCN1CCC(Nc2cccc3c(CC(F)(F)F)c(C#CCNc4cc(F)c(N5CCOCC5)cc4OCC#N)sc23)CC1] 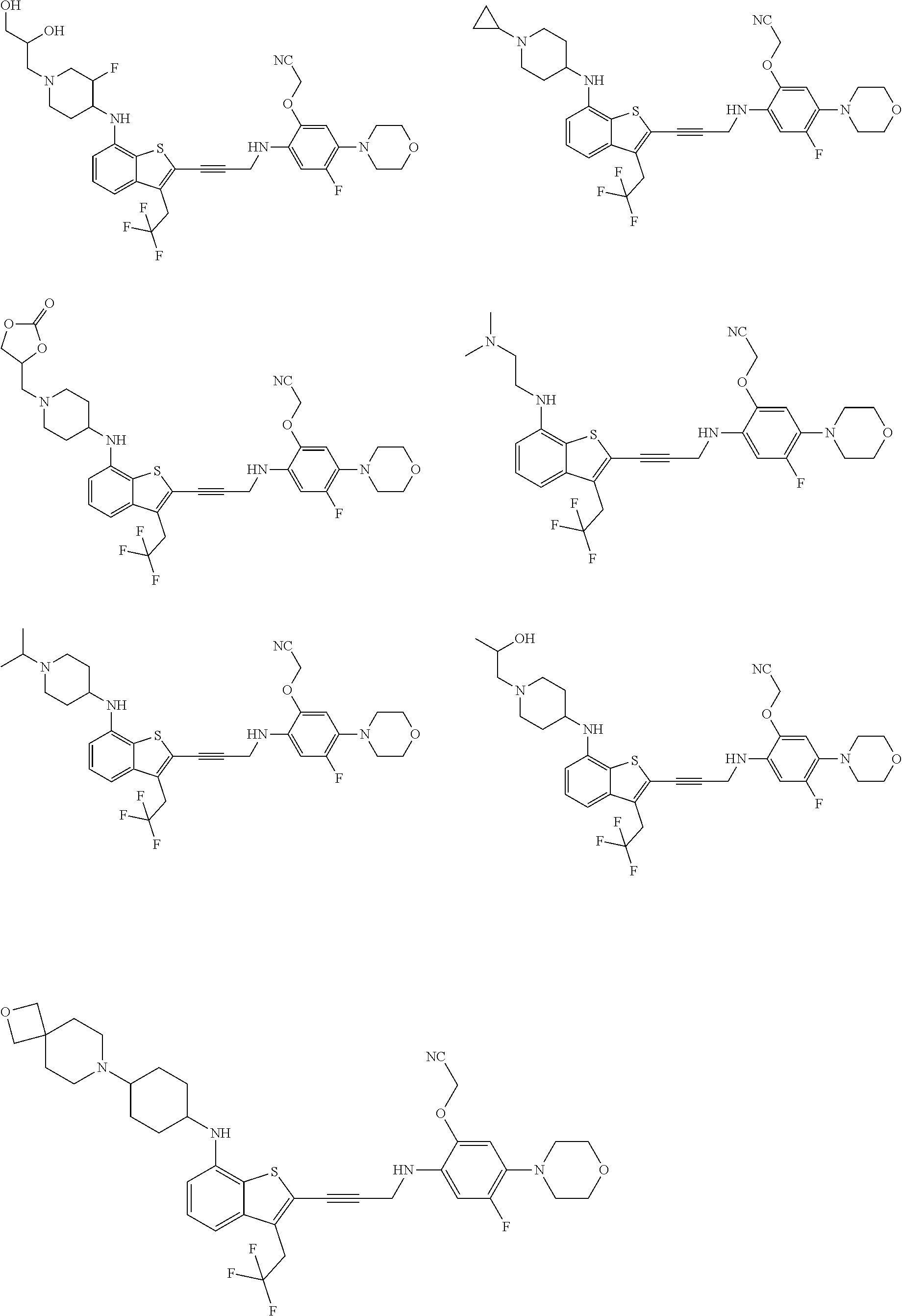 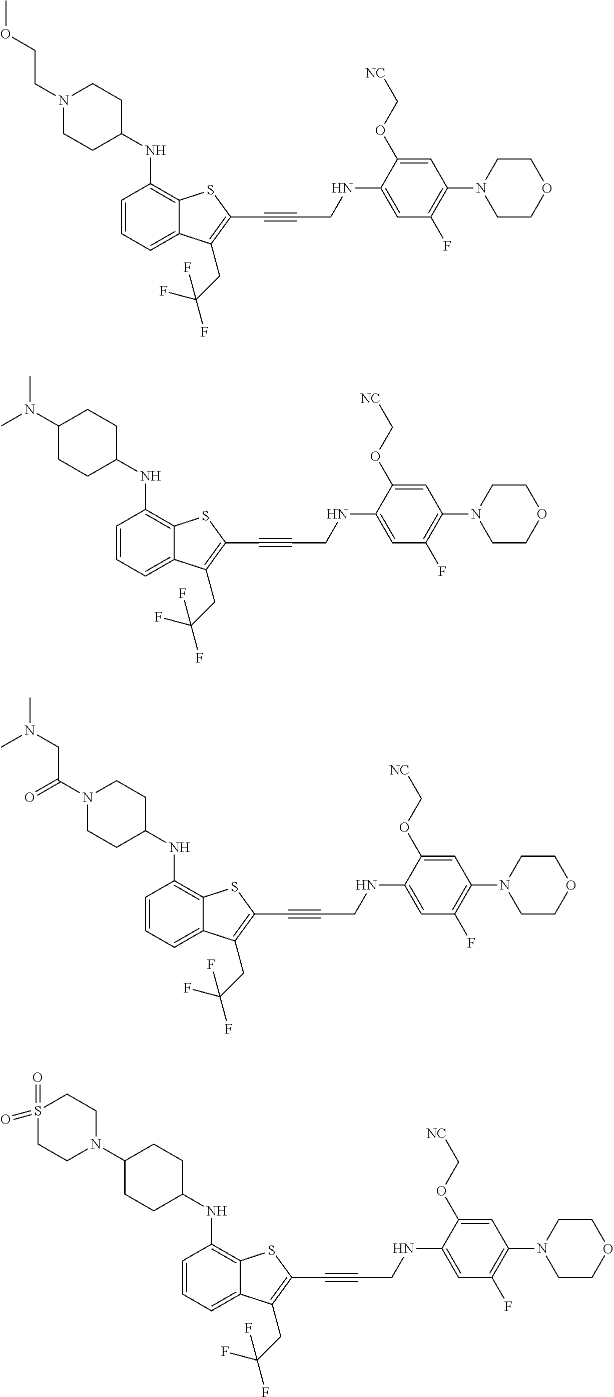 [SMILES: COCCN1CCC(Nc2cccc3c(CC(F)(F)F)c(C#CCNc4cc(F)c(N5CCOCC5)cc4OCC#N)sc23)CC1 ; CN(C)C1CCC(Nc2cccc3c(CC(F)(F)F)c(C#CCNc4cc(F)c(N5CCOCC5)cc4OCC#N)sc23)CC1 ; CN(C)CC(=O)N1CCC(Nc2cccc3c(CC(F)(F)F)c(C#CCNc4cc(F)c(N5CCOCC5)cc4OCC#N)sc23)CC1 ; N#CCOc1cc(N2CCOCC2)c(F)cc1NCC#Cc1sc2c(NC3CCC(N4CCS(=O)(=O)CC4)CC3)cccc2c1CC(F)(F)F] 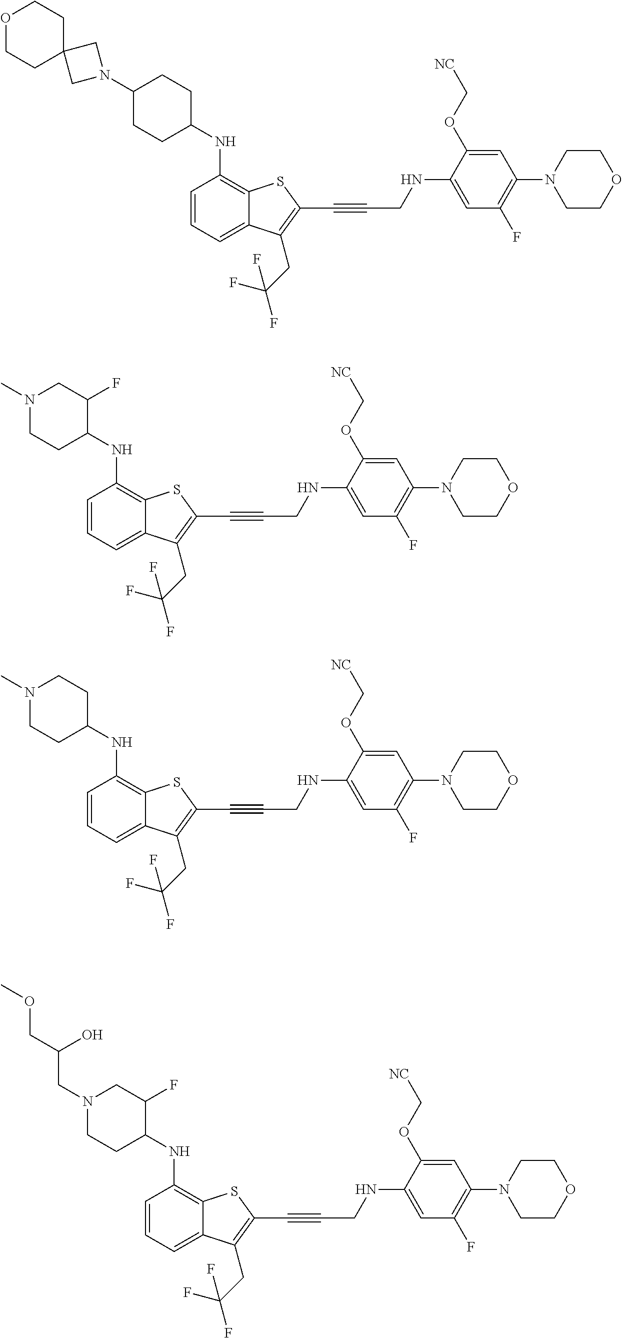 [SMILES: N#CCOc1cc(N2CCOCC2)c(F)cc1NCC#Cc1sc2c(NC3CCC(N4CC5(CCOCC5)C4)CC3)cccc2c1CC(F)(F)F ; CN1CCC(Nc2cccc3c(CC(F)(F)F)c(C#CCNc4cc(F)c(N5CCOCC5)cc4OCC#N)sc23)C(F)C1 ; CN1CCC(Nc2cccc3c(CC(F)(F)F)c(C#CCNc4cc(F)c(N5CCOCC5)cc4OCC#N)sc23)CC1 ; COCC(O)CN1CCC(Nc2cccc3c(CC(F)(F)F)c(C#CCNc4cc(F)c(N5CCOCC5)cc4OCC#N)sc23)C(F)C1] 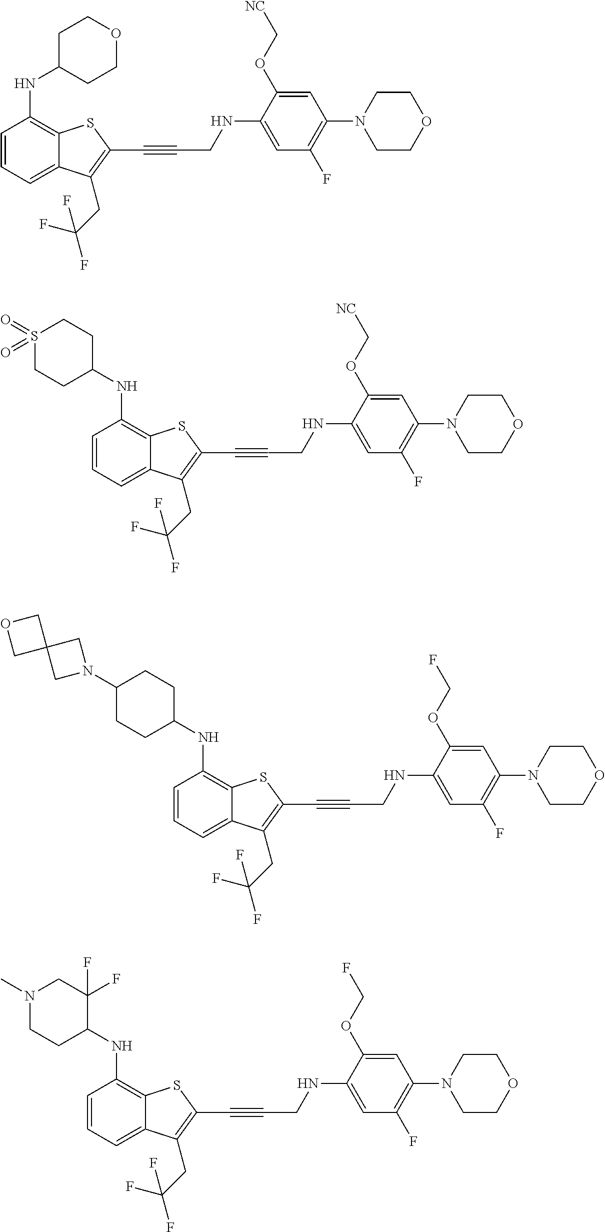 [SMILES: N#CCOc1cc(N2CCOCC2)c(F)cc1NCC#Cc1sc2c(NC3CCOCC3)cccc2c1CC(F)(F)F ; N#CCOc1cc(N2CCOCC2)c(F)cc1NCC#Cc1sc2c(NC3CCS(=O)(=O)CC3)cccc2c1CC(F)(F)F ; FCOc1cc(N2CCOCC2)c(F)cc1NCC#Cc1sc2c(NC3CCC(N4CC5(COC5)C4)CC3)cccc2c1CC(F)(F)F ; CN1CCC(Nc2cccc3c(CC(F)(F)F)c(C#CCNc4cc(F)c(N5CCOCC5)cc4OCF)sc23)C(F)(F)C1] 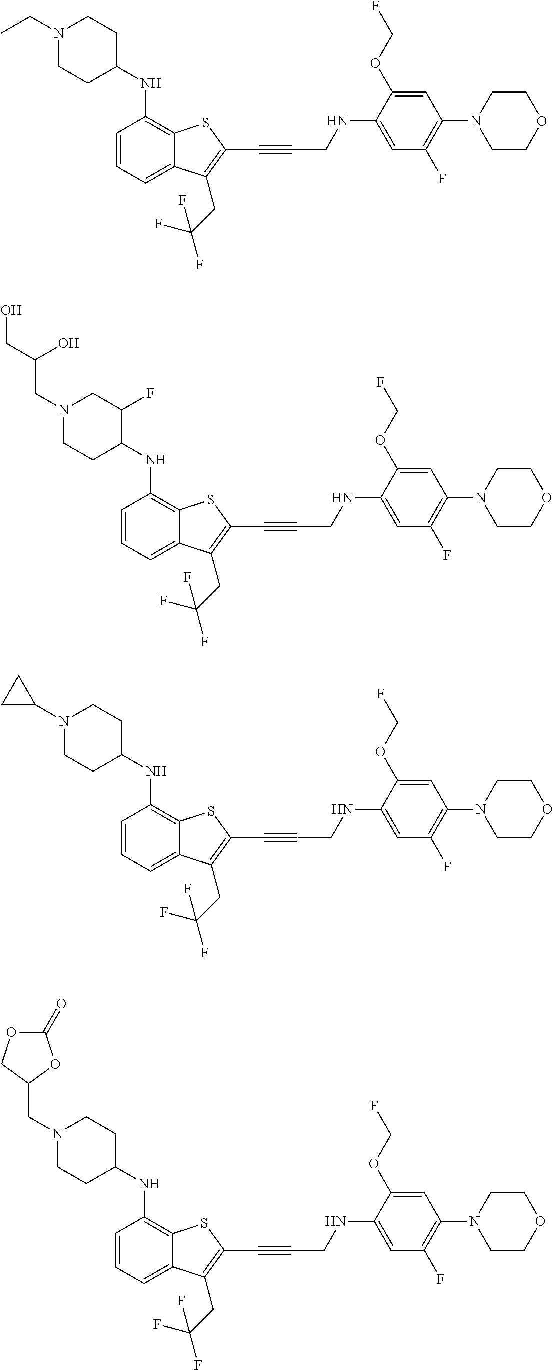 [SMILES: CCN1CCC(Nc2cccc3c(CC(F)(F)F)c(C#CCNc4cc(F)c(N5CCOCC5)cc4OCF)sc23)CC1 ; OCC(O)CN1CCC(Nc2cccc3c(CC(F)(F)F)c(C#CCNc4cc(F)c(N5CCOCC5)cc4OCF)sc23)C(F)C1 ; FCOc1cc(N2CCOCC2)c(F)cc1NCC#Cc1sc2c(NC3CCN(C4CC4)CC3)cccc2c1CC(F)(F)F ; O=C1OCC(CN2CCC(Nc3cccc4c(CC(F)(F)F)c(C#CCNc5cc(F)c(N6CCOCC6)cc5OCF)sc34)CC2)O1] 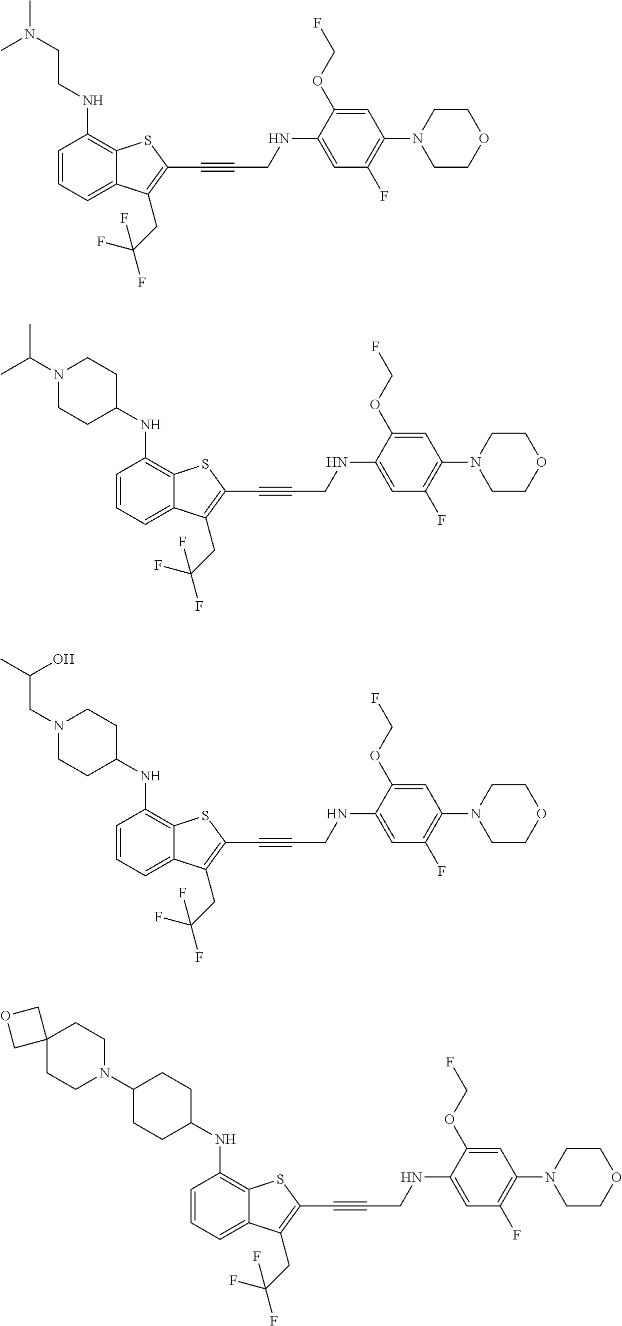 [SMILES: CN(C)CCNc1cccc2c(CC(F)(F)F)c(C#CCNc3cc(F)c(N4CCOCC4)cc3OCF)sc12 ; CC(C)N1CCC(Nc2cccc3c(CC(F)(F)F)c(C#CCNc4cc(F)c(N5CCOCC5)cc4OCF)sc23)CC1 ; CC(O)CN1CCC(Nc2cccc3c(CC(F)(F)F)c(C#CCNc4cc(F)c(N5CCOCC5)cc4OCF)sc23)CC1 ; FCOc1cc(N2CCOCC2)c(F)cc1NCC#Cc1sc2c(NC3CCC(N4CCC5(CC4)COC5)CC3)cccc2c1CC(F)(F)F] 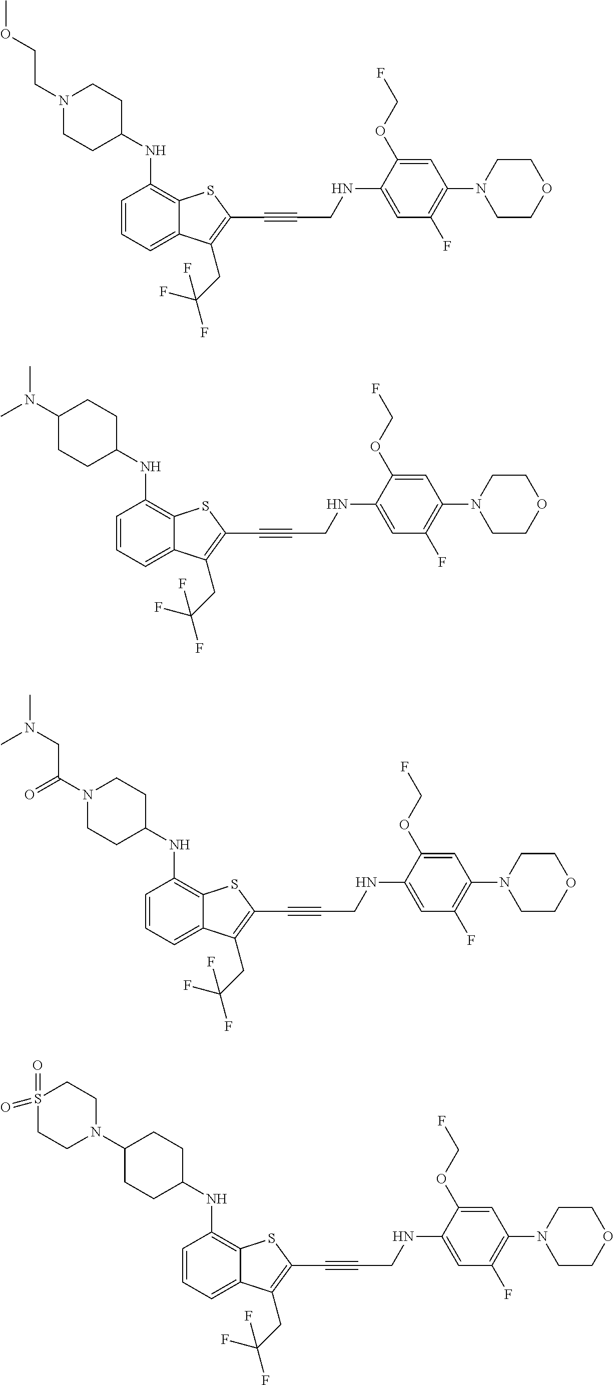 [SMILES: COCCN1CCC(Nc2cccc3c(CC(F)(F)F)c(C#CCNc4cc(F)c(N5CCOCC5)cc4OCF)sc23)CC1 ; CN(C)C1CCC(Nc2cccc3c(CC(F)(F)F)c(C#CCNc4cc(F)c(N5CCOCC5)cc4OCF)sc23)CC1 ; CN(C)CC(=O)N1CCC(Nc2cccc3c(CC(F)(F)F)c(C#CCNc4cc(F)c(N5CCOCC5)cc4OCF)sc23)CC1 ; O=S1(=O)CCN(C2CCC(Nc3cccc4c(CC(F)(F)F)c(C#CCNc5cc(F)c(N6CCOCC6)cc5OCF)sc34)CC2)CC1] 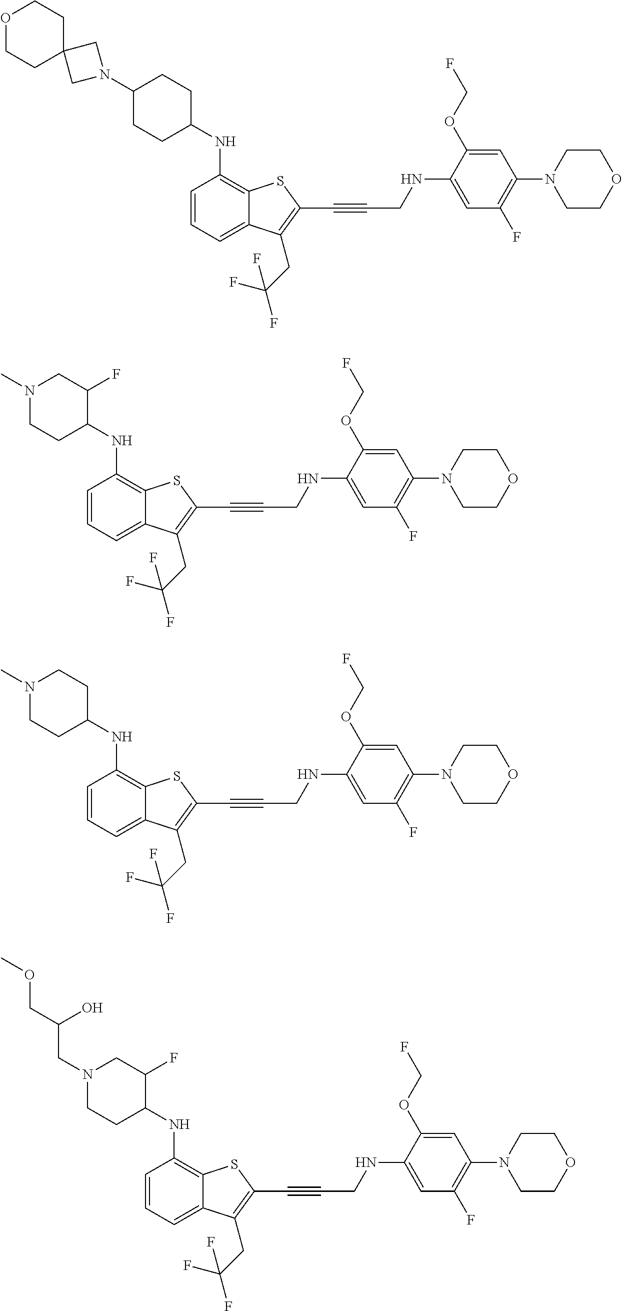 [SMILES: FCOc1cc(N2CCOCC2)c(F)cc1NCC#Cc1sc2c(NC3CCC(N4CC5(CCOCC5)C4)CC3)cccc2c1CC(F)(F)F ; CN1CCC(Nc2cccc3c(CC(F)(F)F)c(C#CCNc4cc(F)c(N5CCOCC5)cc4OCF)sc23)C(F)C1 ; CN1CCC(Nc2cccc3c(CC(F)(F)F)c(C#CCNc4cc(F)c(N5CCOCC5)cc4OCF)sc23)CC1 ; COCC(O)CN1CCC(Nc2cccc3c(CC(F)(F)F)c(C#CCNc4cc(F)c(N5CCOCC5)cc4OCF)sc23)C(F)C1] 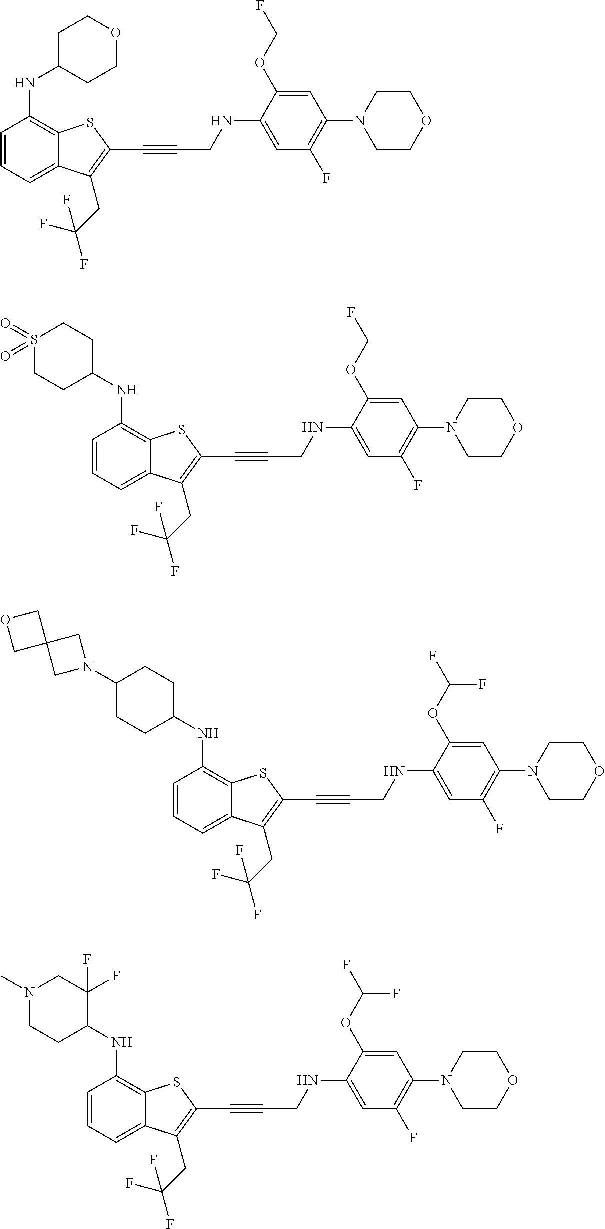 [SMILES: FCOc1cc(N2CCOCC2)c(F)cc1NCC#Cc1sc2c(NC3CCOCC3)cccc2c1CC(F)(F)F ; O=S1(=O)CCC(Nc2cccc3c(CC(F)(F)F)c(C#CCNc4cc(F)c(N5CCOCC5)cc4OCF)sc23)CC1 ; Fc1cc(NCC#Cc2sc3c(NC4CCC(N5CC6(COC6)C5)CC4)cccc3c2CC(F)(F)F)c(OC(F)F)cc1N1CCOCC1 ; CN1CCC(Nc2cccc3c(CC(F)(F)F)c(C#CCNc4cc(F)c(N5CCOCC5)cc4OC(F)F)sc23)C(F)(F)C1] 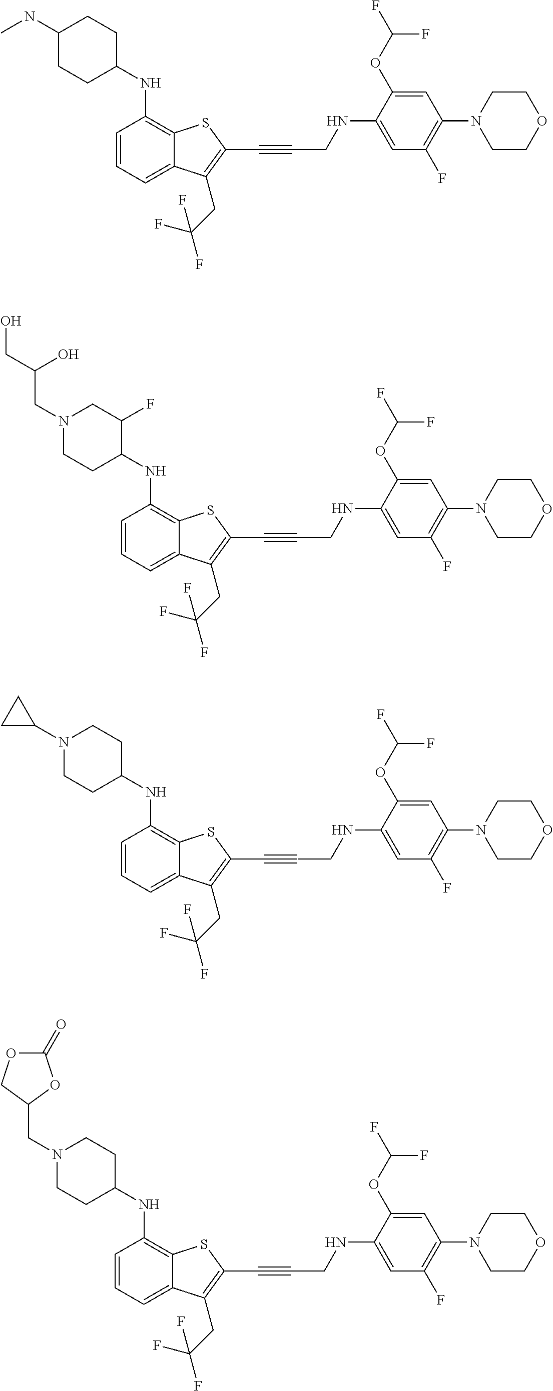 [SMILES: CNC1CCC(Nc2cccc3c(CC(F)(F)F)c(C#CCNc4cc(F)c(N5CCOCC5)cc4OC(F)F)sc23)CC1 ; OCC(O)CN1CCC(Nc2cccc3c(CC(F)(F)F)c(C#CCNc4cc(F)c(N5CCOCC5)cc4OC(F)F)sc23)C(F)C1 ; Fc1cc(NCC#Cc2sc3c(NC4CCN(C5CC5)CC4)cccc3c2CC(F)(F)F)c(OC(F)F)cc1N1CCOCC1 ; O=C1OCC(CN2CCC(Nc3cccc4c(CC(F)(F)F)c(C#CCNc5cc(F)c(N6CCOCC6)cc5OC(F)F)sc34)CC2)O1] 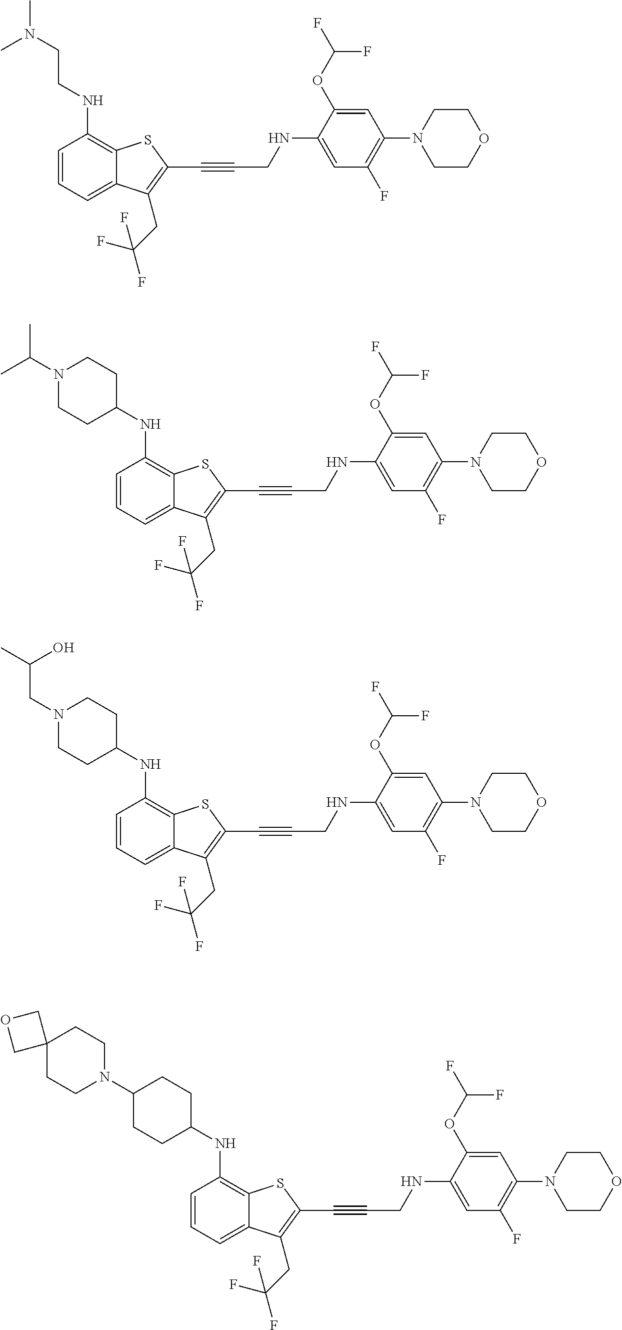 [SMILES: CN(C)CCNc1cccc2c(CC(F)(F)F)c(C#CCNc3cc(F)c(N4CCOCC4)cc3OC(F)F)sc12 ; CC(C)N1CCC(Nc2cccc3c(CC(F)(F)F)c(C#CCNc4cc(F)c(N5CCOCC5)cc4OC(F)F)sc23)CC1 ; CC(O)CN1CCC(Nc2cccc3c(CC(F)(F)F)c(C#CCNc4cc(F)c(N5CCOCC5)cc4OC(F)F)sc23)CC1 ; Fc1cc(NCC#Cc2sc3c(NC4CCC(N5CCC6(CC5)COC6)CC4)cccc3c2CC(F)(F)F)c(OC(F)F)cc1N1CCOCC1] 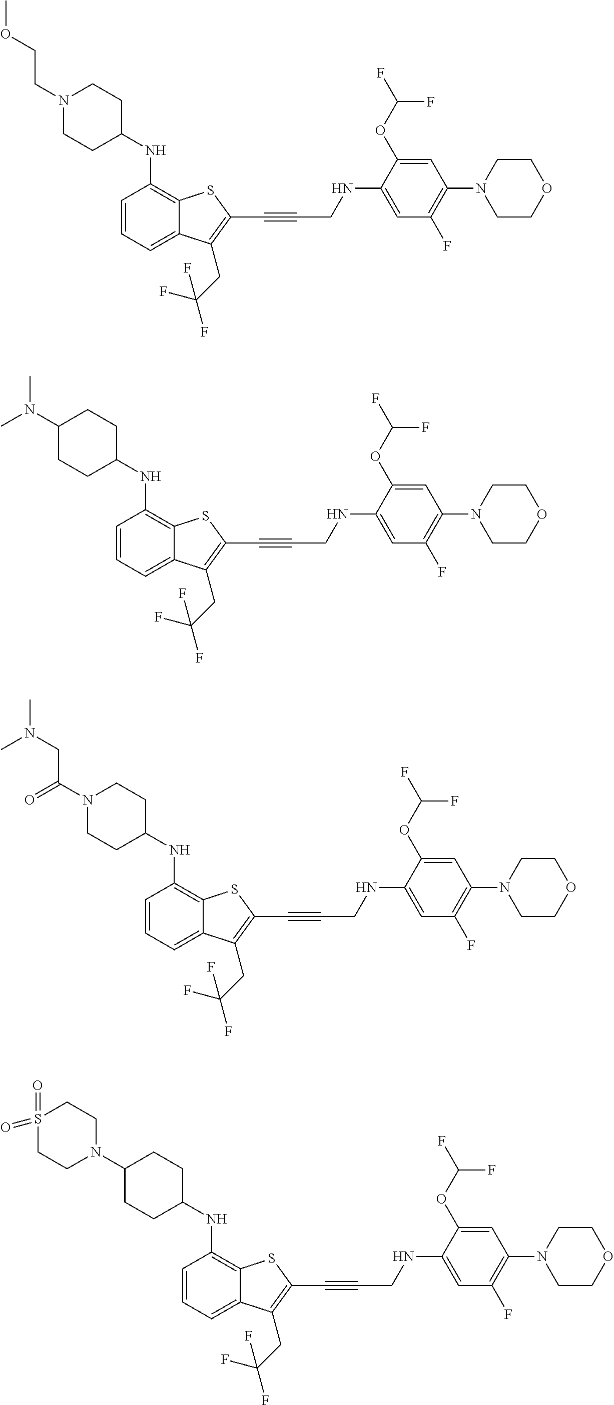 [SMILES: COCCN1CCC(Nc2cccc3c(CC(F)(F)F)c(C#CCNc4cc(F)c(N5CCOCC5)cc4OC(F)F)sc23)CC1 ; CN(C)C1CCC(Nc2cccc3c(CC(F)(F)F)c(C#CCNc4cc(F)c(N5CCOCC5)cc4OC(F)F)sc23)CC1 ; CN(C)CC(=O)N1CCC(Nc2cccc3c(CC(F)(F)F)c(C#CCNc4cc(F)c(N5CCOCC5)cc4OC(F)F)sc23)CC1 ; O=S1(=O)CCN(C2CCC(Nc3cccc4c(CC(F)(F)F)c(C#CCNc5cc(F)c(N6CCOCC6)cc5OC(F)F)sc34)CC2)CC1] 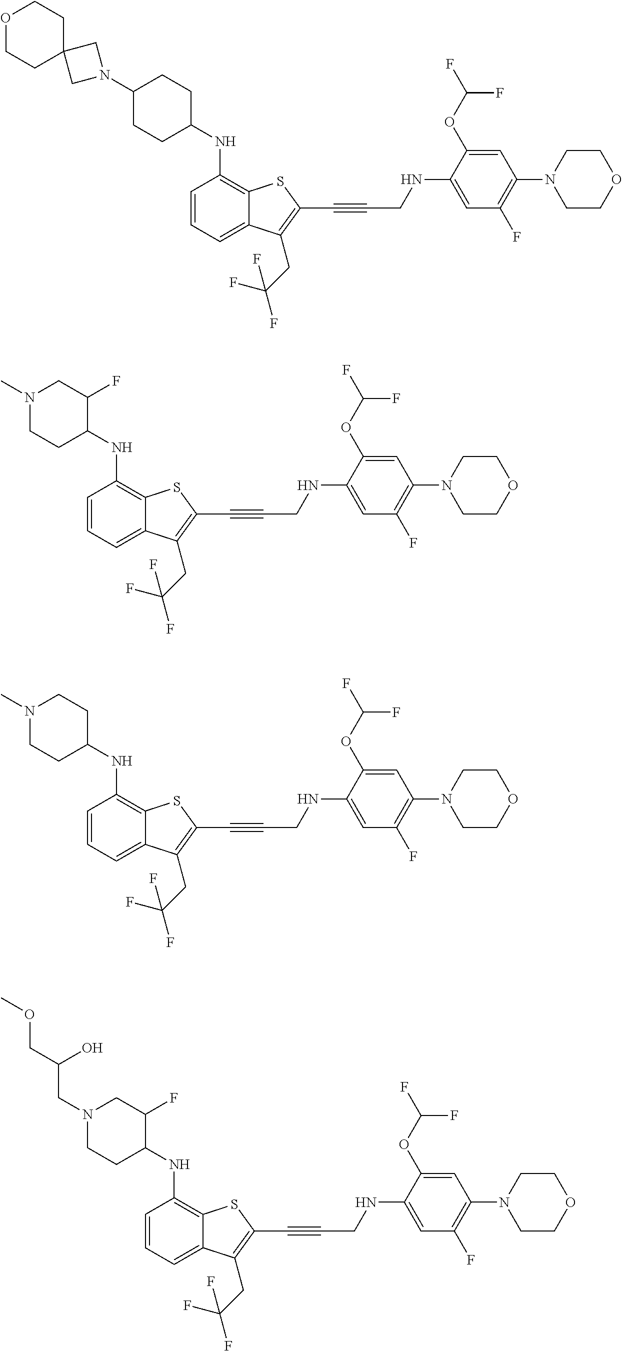 [SMILES: Fc1cc(NCC#Cc2sc3c(NC4CCC(N5CC6(CCOCC6)C5)CC4)cccc3c2CC(F)(F)F)c(OC(F)F)cc1N1CCOCC1 ; CN1CCC(Nc2cccc3c(CC(F)(F)F)c(C#CCNc4cc(F)c(N5CCOCC5)cc4OC(F)F)sc23)C(F)C1 ; CN1CCC(Nc2cccc3c(CC(F)(F)F)c(C#CCNc4cc(F)c(N5CCOCC5)cc4OC(F)F)sc23)CC1 ; COCC(O)CN1CCC(Nc2cccc3c(CC(F)(F)F)c(C#CCNc4cc(F)c(N5CCOCC5)cc4OC(F)F)sc23)C(F)C1] 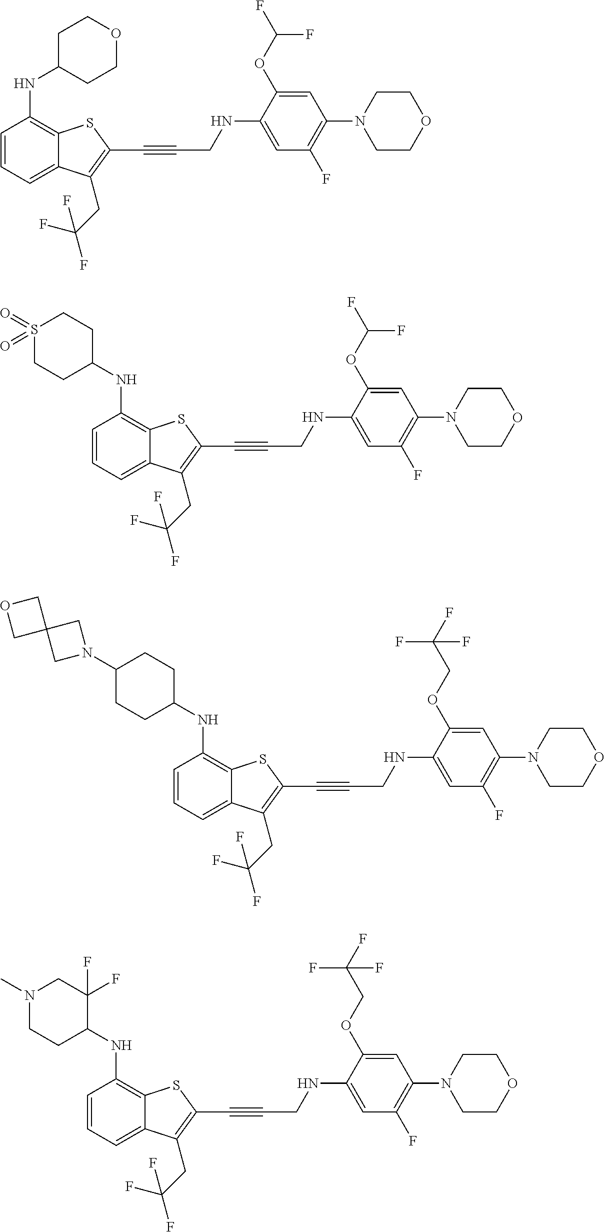 [SMILES: Fc1cc(NCC#Cc2sc3c(NC4CCOCC4)cccc3c2CC(F)(F)F)c(OC(F)F)cc1N1CCOCC1 ; O=S1(=O)CCC(Nc2cccc3c(CC(F)(F)F)c(C#CCNc4cc(F)c(N5CCOCC5)cc4OC(F)F)sc23)CC1 ; Fc1cc(NCC#Cc2sc3c(NC4CCC(N5CC6(COC6)C5)CC4)cccc3c2CC(F)(F)F)c(OCC(F)(F)F)cc1N1CCOCC1 ; CN1CCC(Nc2cccc3c(CC(F)(F)F)c(C#CCNc4cc(F)c(N5CCOCC5)cc4OCC(F)(F)F)sc23)C(F)(F)C1] 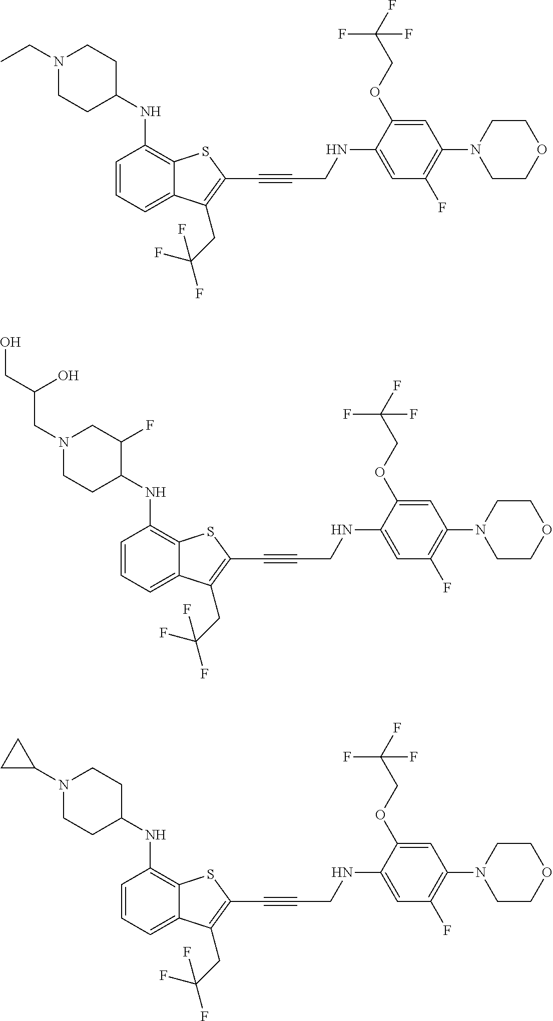 [SMILES: CCN1CCC(Nc2cccc3c(CC(F)(F)F)c(C#CCNc4cc(F)c(N5CCOCC5)cc4OCC(F)(F)F)sc23)CC1 ; OCC(O)CN1CCC(Nc2cccc3c(CC(F)(F)F)c(C#CCNc4cc(F)c(N5CCOCC5)cc4OCC(F)(F)F)sc23)C(F)C1 ; Fc1cc(NCC#Cc2sc3c(NC4CCN(C5CC5)CC4)cccc3c2CC(F)(F)F)c(OCC(F)(F)F)cc1N1CCOCC1] 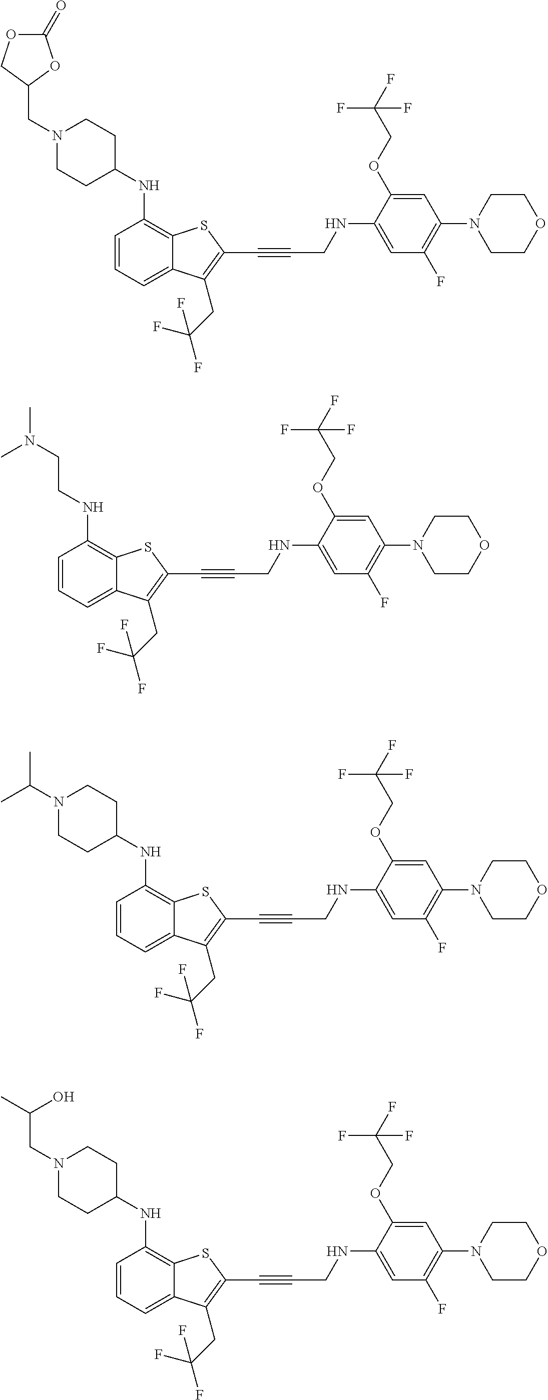 [SMILES: O=C1OCC(CN2CCC(Nc3cccc4c(CC(F)(F)F)c(C#CCNc5cc(F)c(N6CCOCC6)cc5OCC(F)(F)F)sc34)CC2)O1 ; CN(C)CCNc1cccc2c(CC(F)(F)F)c(C#CCNc3cc(F)c(N4CCOCC4)cc3OCC(F)(F)F)sc12 ; CC(C)N1CCC(Nc2cccc3c(CC(F)(F)F)c(C#CCNc4cc(F)c(N5CCOCC5)cc4OCC(F)(F)F)sc23)CC1 ; CC(O)CN1CCC(Nc2cccc3c(CC(F)(F)F)c(C#CCNc4cc(F)c(N5CCOCC5)cc4OCC(F)(F)F)sc23)CC1] 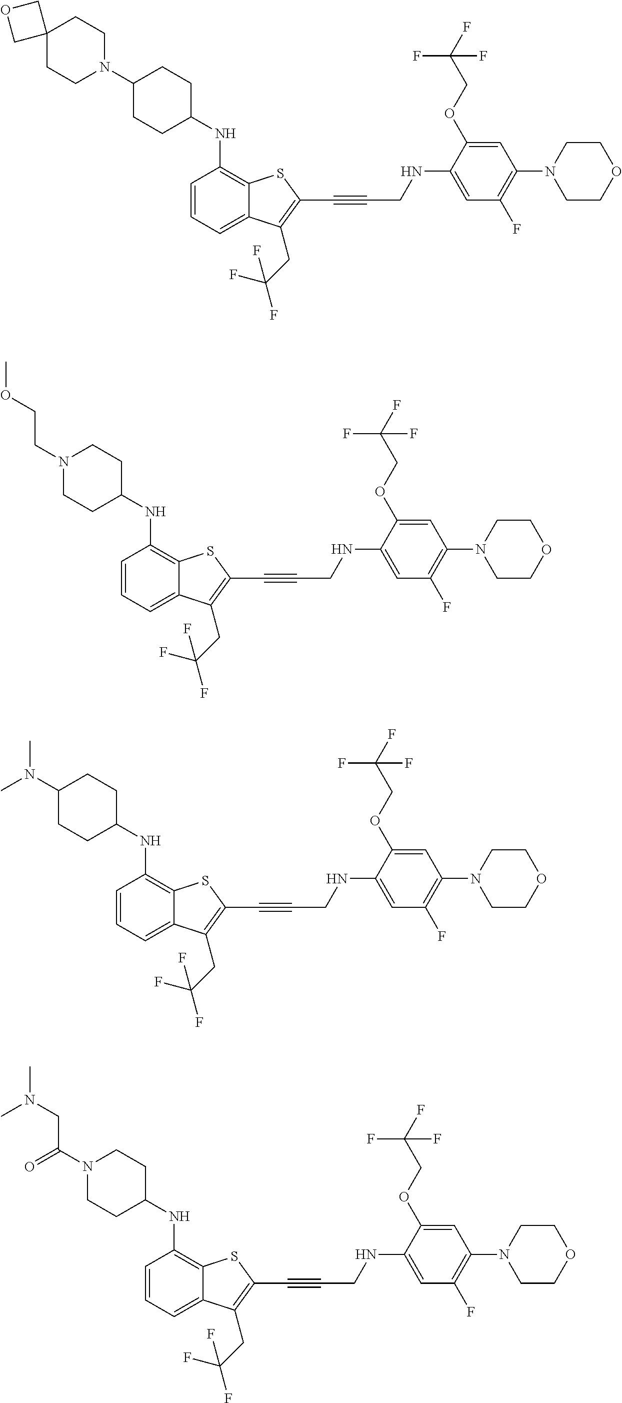 [SMILES: Fc1cc(NCC#Cc2sc3c(NC4CCC(N5CCC6(CC5)COC6)CC4)cccc3c2CC(F)(F)F)c(OCC(F)(F)F)cc1N1CCOCC1 ; COCCN1CCC(Nc2cccc3c(CC(F)(F)F)c(C#CCNc4cc(F)c(N5CCOCC5)cc4OCC(F)(F)F)sc23)CC1 ; CN(C)C1CCC(Nc2cccc3c(CC(F)(F)F)c(C#CCNc4cc(F)c(N5CCOCC5)cc4OCC(F)(F)F)sc23)CC1 ; CN(C)CC(=O)N1CCC(Nc2cccc3c(CC(F)(F)F)c(C#CCNc4cc(F)c(N5CCOCC5)cc4OCC(F)(F)F)sc23)CC1] 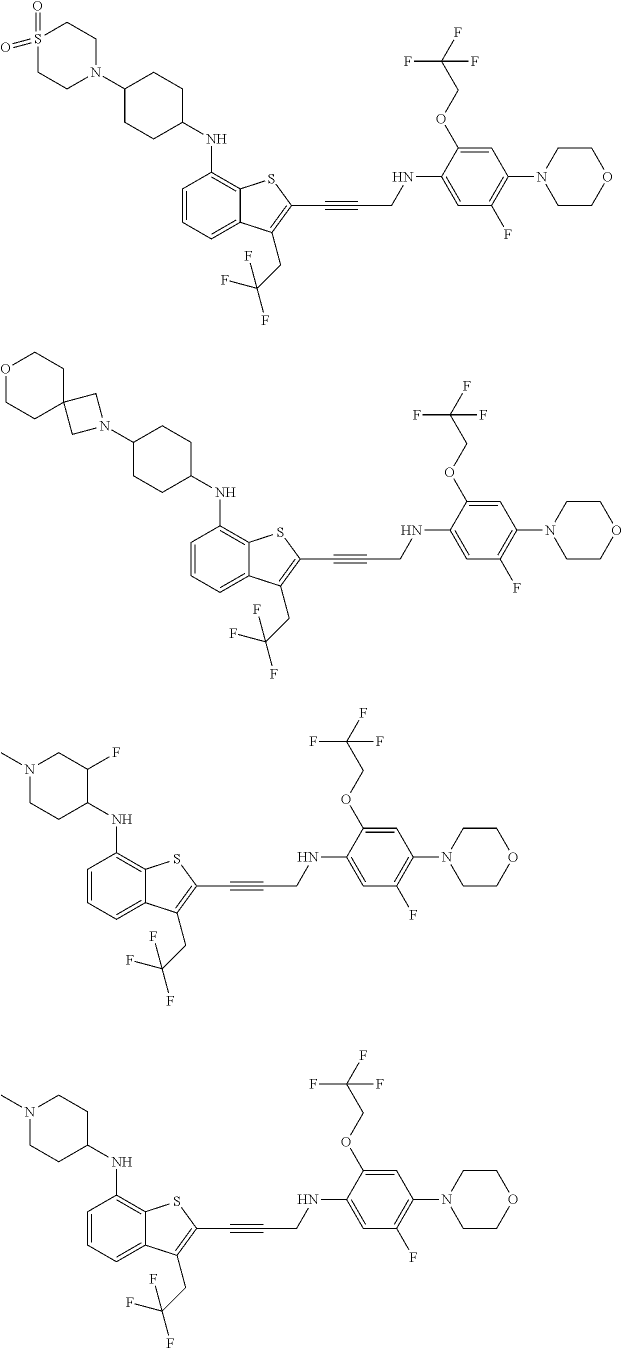 [SMILES: O=S1(=O)CCN(C2CCC(Nc3cccc4c(CC(F)(F)F)c(C#CCNc5cc(F)c(N6CCOCC6)cc5OCC(F)(F)F)sc34)CC2)CC1 ; Fc1cc(NCC#Cc2sc3c(NC4CCC(N5CC6(CCOCC6)C5)CC4)cccc3c2CC(F)(F)F)c(OCC(F)(F)F)cc1N1CCOCC1 ; CN1CCC(Nc2cccc3c(CC(F)(F)F)c(C#CCNc4cc(F)c(N5CCOCC5)cc4OCC(F)(F)F)sc23)C(F)C1 ; CN1CCC(Nc2cccc3c(CC(F)(F)F)c(C#CCNc4cc(F)c(N5CCOCC5)cc4OCC(F)(F)F)sc23)CC1] 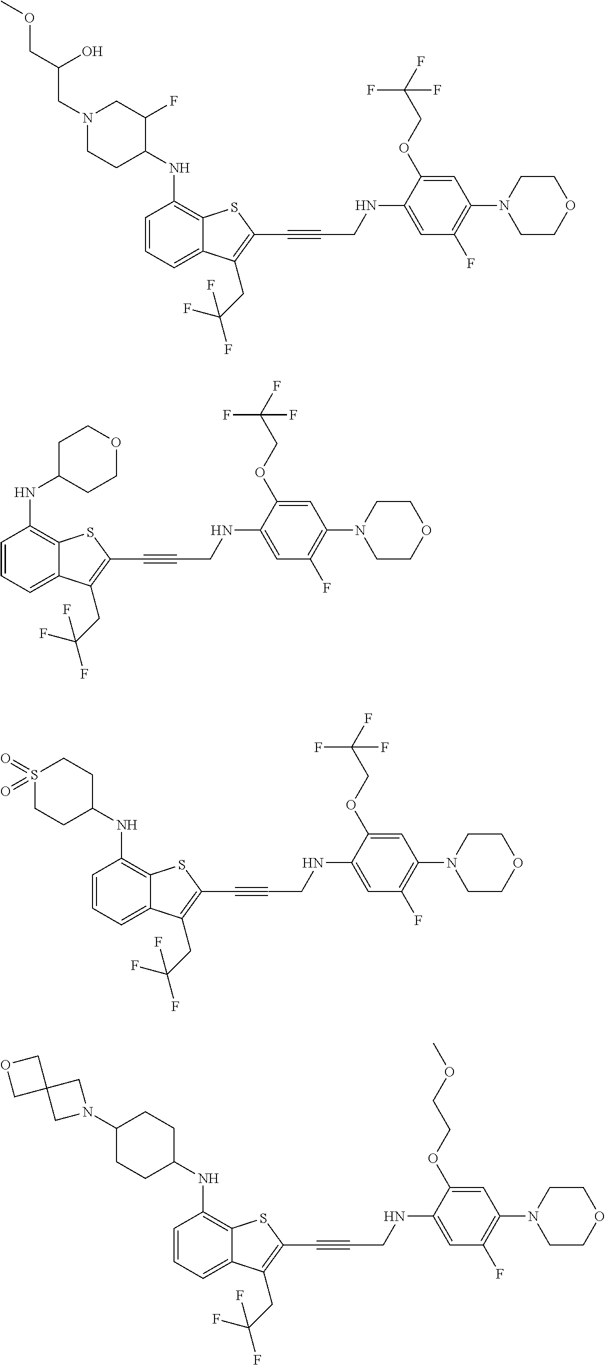 [SMILES: COCC(O)CN1CCC(Nc2cccc3c(CC(F)(F)F)c(C#CCNc4cc(F)c(N5CCOCC5)cc4OCC(F)(F)F)sc23)C(F)C1 ; Fc1cc(NCC#Cc2sc3c(NC4CCOCC4)cccc3c2CC(F)(F)F)c(OCC(F)(F)F)cc1N1CCOCC1 ; O=S1(=O)CCC(Nc2cccc3c(CC(F)(F)F)c(C#CCNc4cc(F)c(N5CCOCC5)cc4OCC(F)(F)F)sc23)CC1 ; COCCOc1cc(N2CCOCC2)c(F)cc1NCC#Cc1sc2c(NC3CCC(N4CC5(COC5)C4)CC3)cccc2c1CC(F)(F)F] 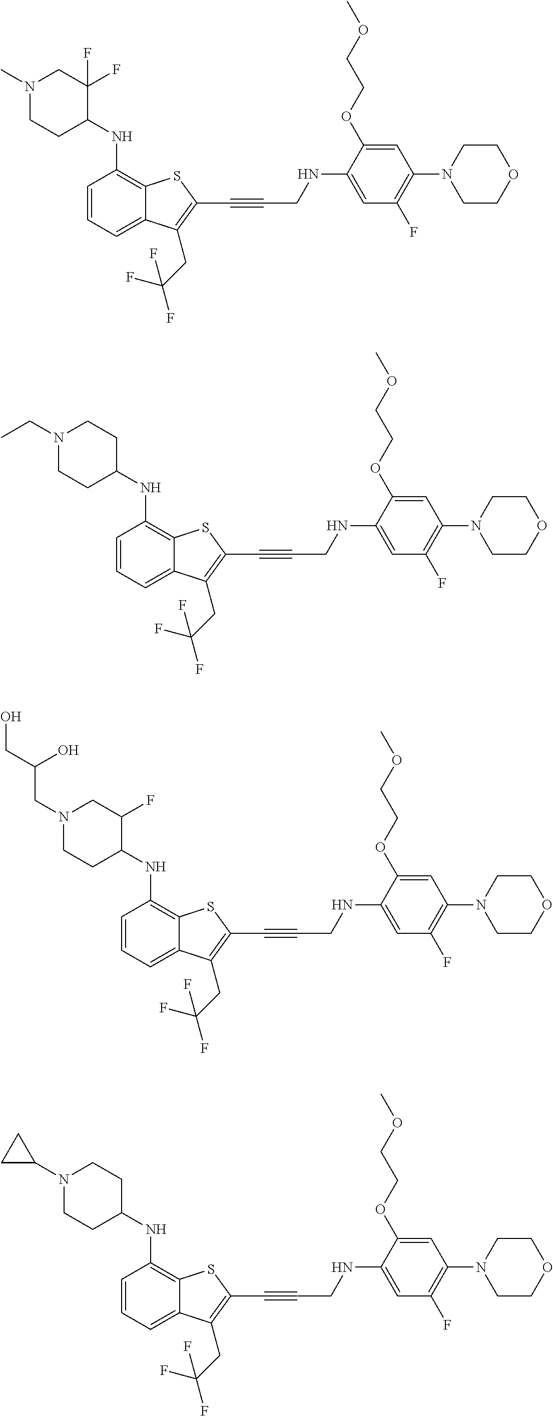 [SMILES: COCCOc1cc(N2CCOCC2)c(F)cc1NCC#Cc1sc2c(NC3CCN(C)CC3(F)F)cccc2c1CC(F)(F)F ; CCN1CCC(Nc2cccc3c(CC(F)(F)F)c(C#CCNc4cc(F)c(N5CCOCC5)cc4OCCOC)sc23)CC1 ; COCCOc1cc(N2CCOCC2)c(F)cc1NCC#Cc1sc2c(NC3CCN(CC(O)CO)CC3F)cccc2c1CC(F)(F)F ; COCCOc1cc(N2CCOCC2)c(F)cc1NCC#Cc1sc2c(NC3CCN(C4CC4)CC3)cccc2c1CC(F)(F)F] 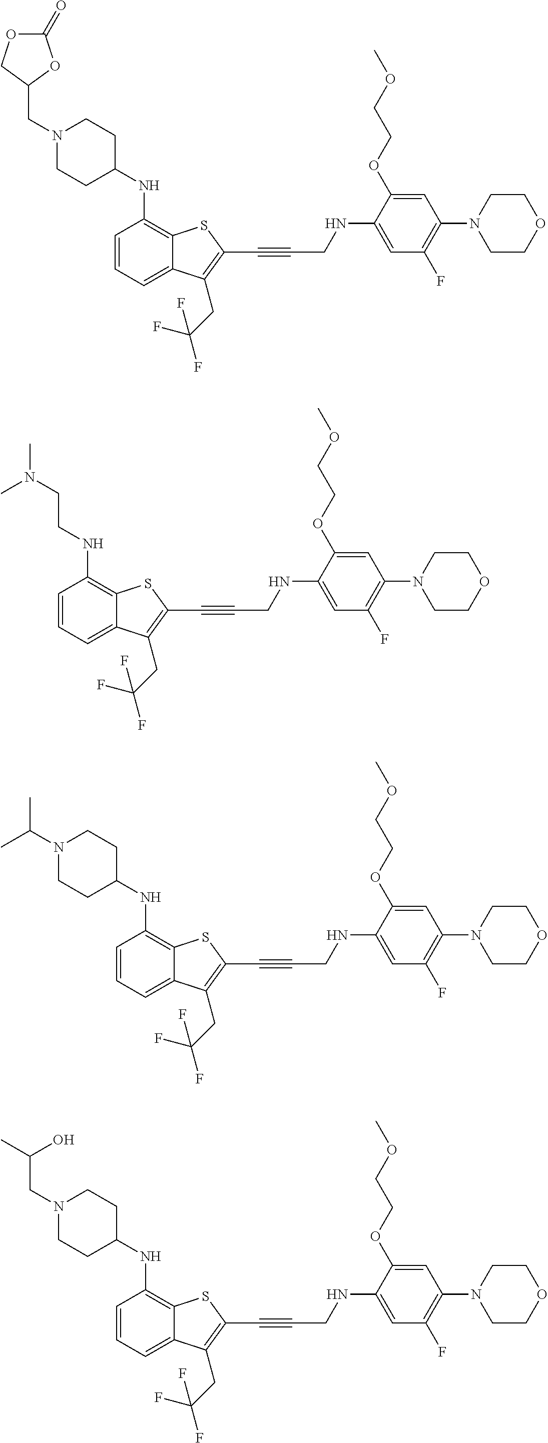 [SMILES: COCCOc1cc(N2CCOCC2)c(F)cc1NCC#Cc1sc2c(NC3CCN(CC4COC(=O)O4)CC3)cccc2c1CC(F)(F)F ; COCCOc1cc(N2CCOCC2)c(F)cc1NCC#Cc1sc2c(NCCN(C)C)cccc2c1CC(F)(F)F ; COCCOc1cc(N2CCOCC2)c(F)cc1NCC#Cc1sc2c(NC3CCN(C(C)C)CC3)cccc2c1CC(F)(F)F ; COCCOc1cc(N2CCOCC2)c(F)cc1NCC#Cc1sc2c(NC3CCN(CC(C)O)CC3)cccc2c1CC(F)(F)F] 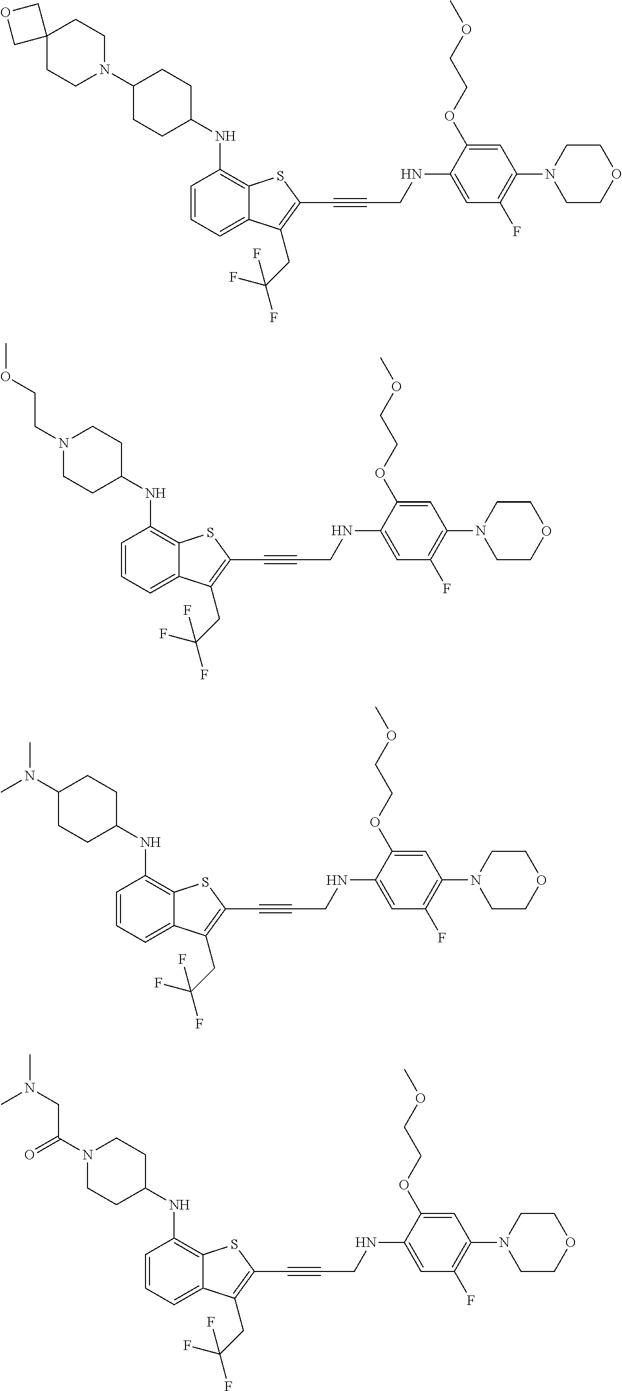 [SMILES: COCCOc1cc(N2CCOCC2)c(F)cc1NCC#Cc1sc2c(NC3CCC(N4CCC5(CC4)COC5)CC3)cccc2c1CC(F)(F)F ; COCCOc1cc(N2CCOCC2)c(F)cc1NCC#Cc1sc2c(NC3CCN(CCOC)CC3)cccc2c1CC(F)(F)F ; COCCOc1cc(N2CCOCC2)c(F)cc1NCC#Cc1sc2c(NC3CCC(N(C)C)CC3)cccc2c1CC(F)(F)F ; COCCOc1cc(N2CCOCC2)c(F)cc1NCC#Cc1sc2c(NC3CCN(C(=O)CN(C)C)CC3)cccc2c1CC(F)(F)F] 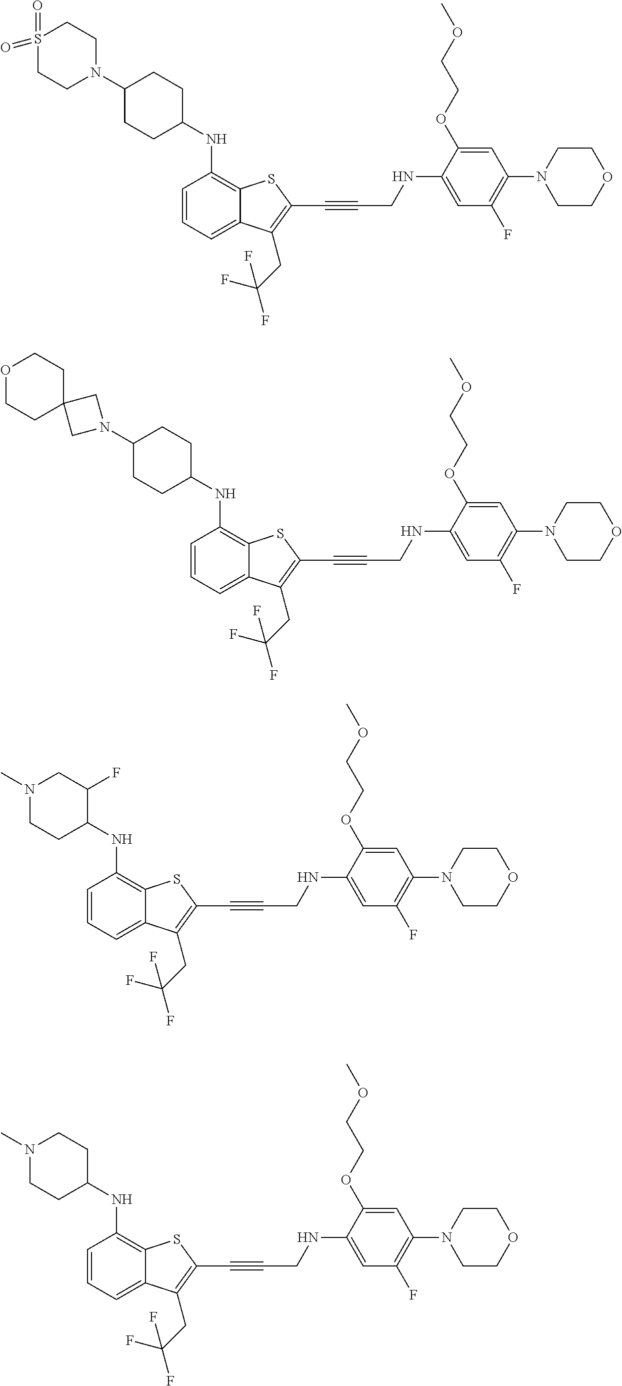 [SMILES: COCCOc1cc(N2CCOCC2)c(F)cc1NCC#Cc1sc2c(NC3CCC(N4CCS(=O)(=O)CC4)CC3)cccc2c1CC(F)(F)F ; COCCOc1cc(N2CCOCC2)c(F)cc1NCC#Cc1sc2c(NC3CCC(N4CC5(CCOCC5)C4)CC3)cccc2c1CC(F)(F)F ; COCCOc1cc(N2CCOCC2)c(F)cc1NCC#Cc1sc2c(NC3CCN(C)CC3F)cccc2c1CC(F)(F)F ; COCCOc1cc(N2CCOCC2)c(F)cc1NCC#Cc1sc2c(NC3CCN(C)CC3)cccc2c1CC(F)(F)F] 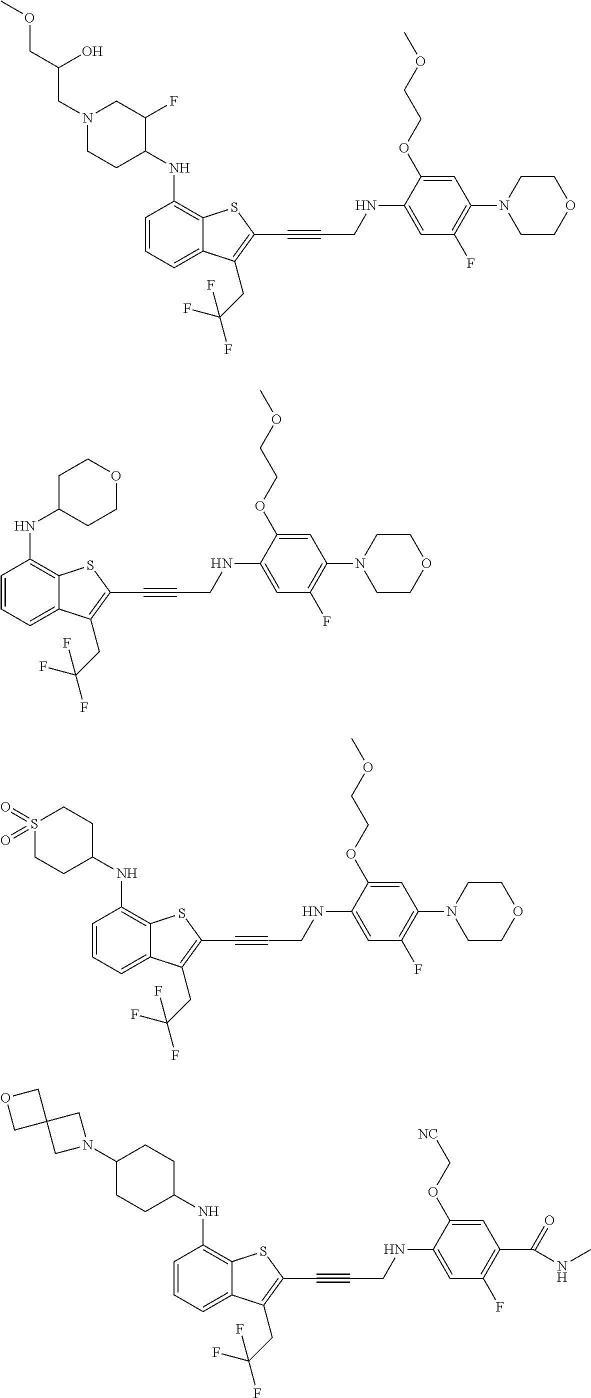 [SMILES: COCCOc1cc(N2CCOCC2)c(F)cc1NCC#Cc1sc2c(NC3CCN(CC(O)COC)CC3F)cccc2c1CC(F)(F)F ; COCCOc1cc(N2CCOCC2)c(F)cc1NCC#Cc1sc2c(NC3CCOCC3)cccc2c1CC(F)(F)F ; COCCOc1cc(N2CCOCC2)c(F)cc1NCC#Cc1sc2c(NC3CCS(=O)(=O)CC3)cccc2c1CC(F)(F)F ; CNC(=O)c1cc(OCC#N)c(NCC#Cc2sc3c(NC4CCC(N5CC6(COC6)C5)CC4)cccc3c2CC(F)(F)F)cc1F] 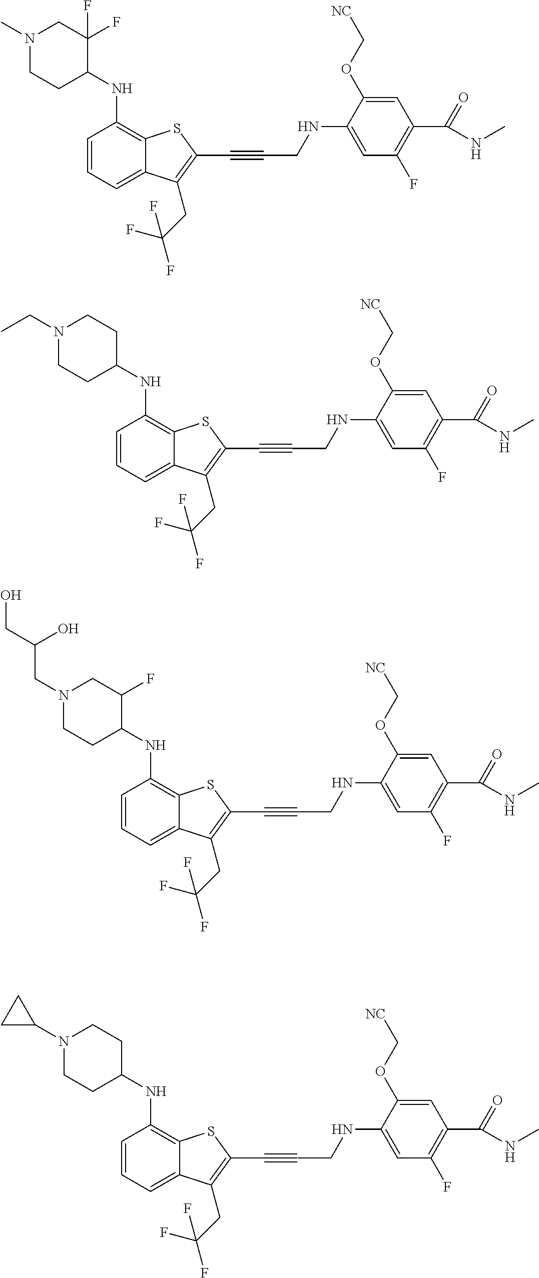 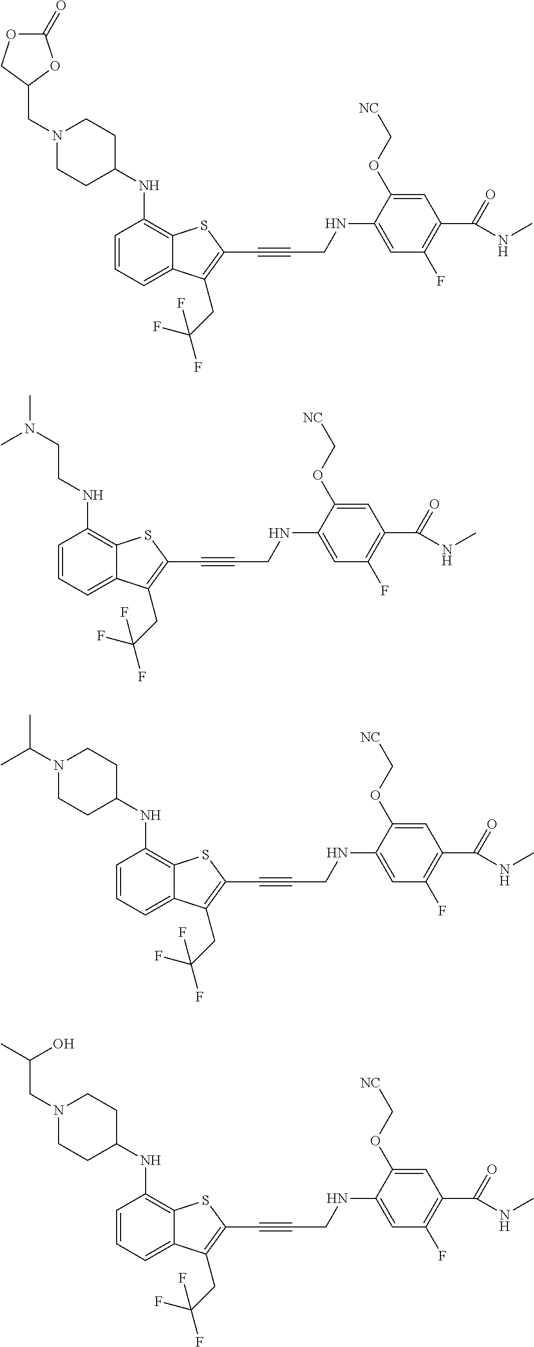 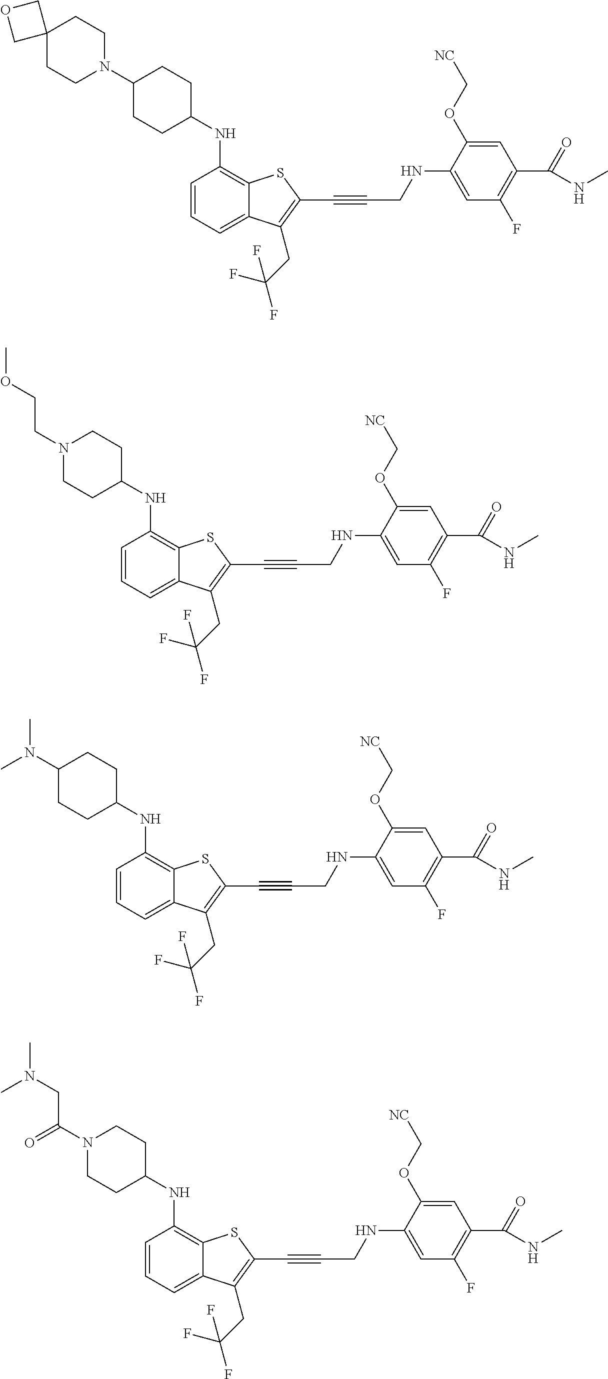 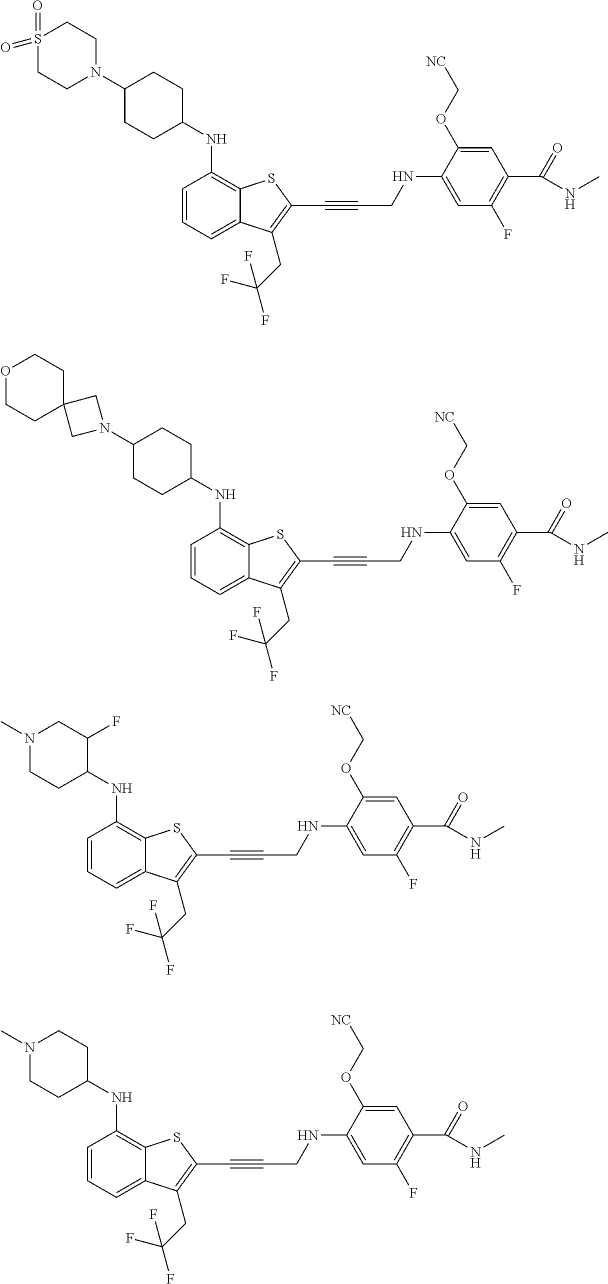 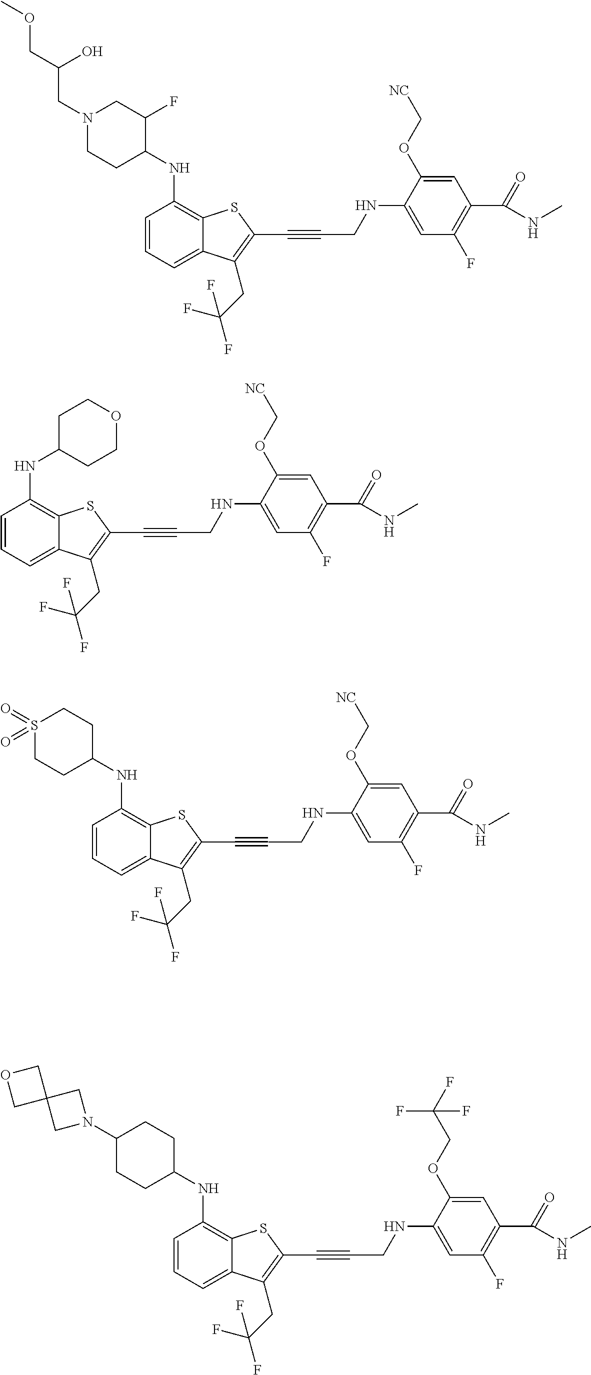 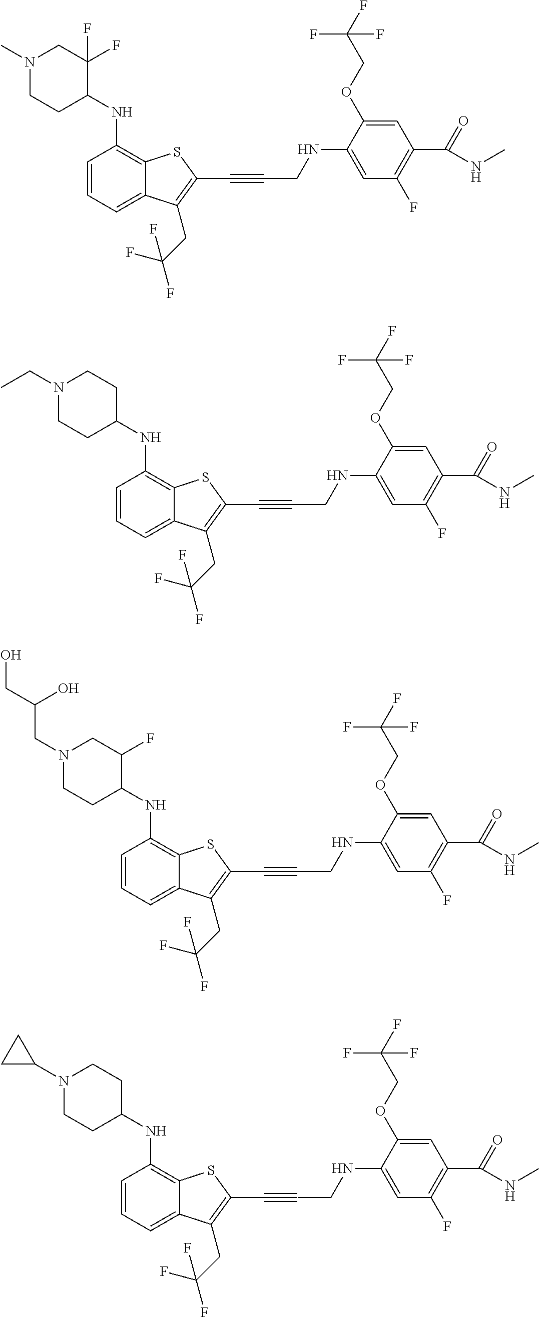 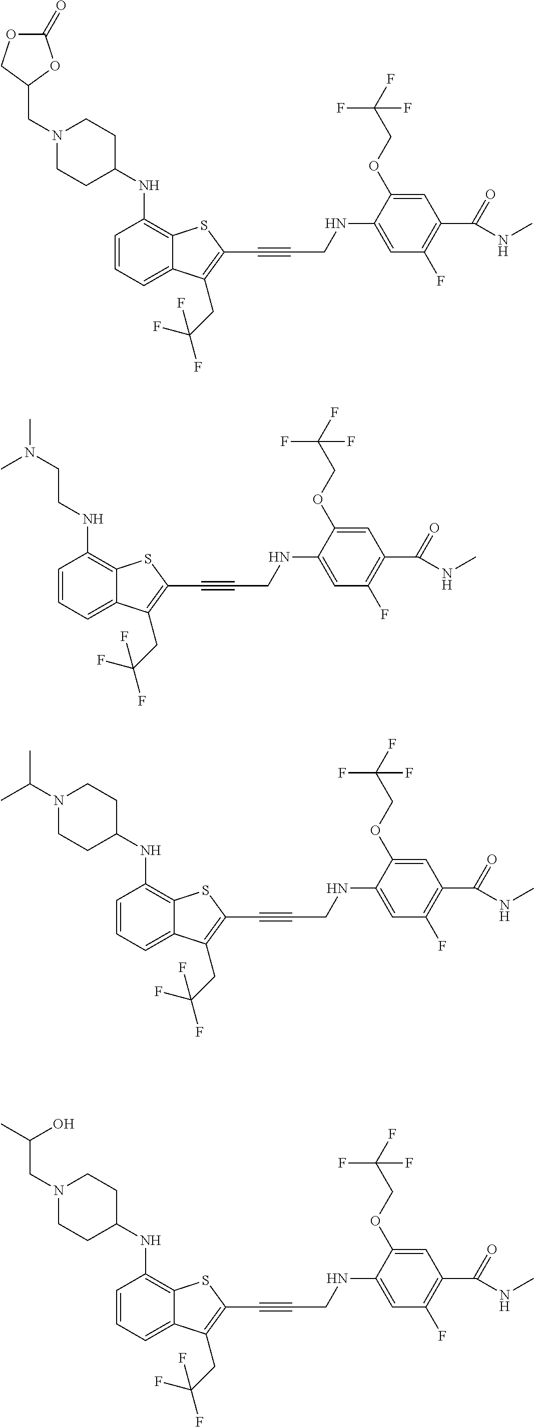 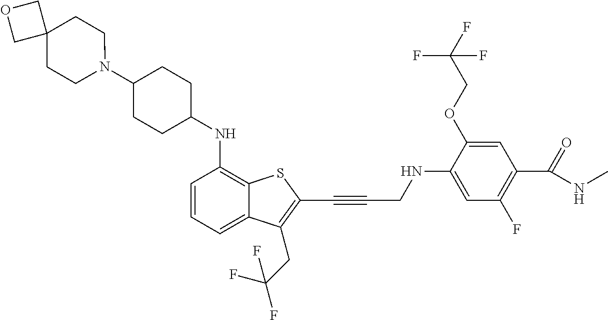 [SMILES: CNC(=O)c1cc(OCC(F)(F)F)c(NCC#Cc2sc3c(NC4CCC(N5CCC6(CC5)COC6)CC4)cccc3c2CC(F)(F)F)cc1F] 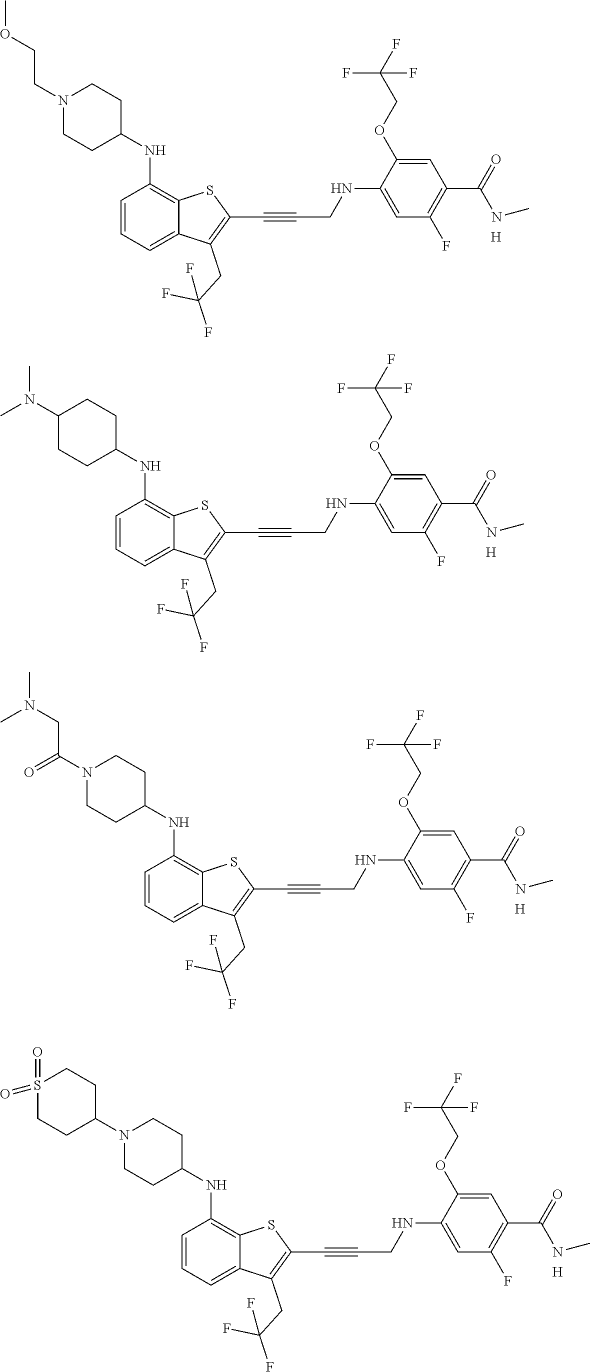 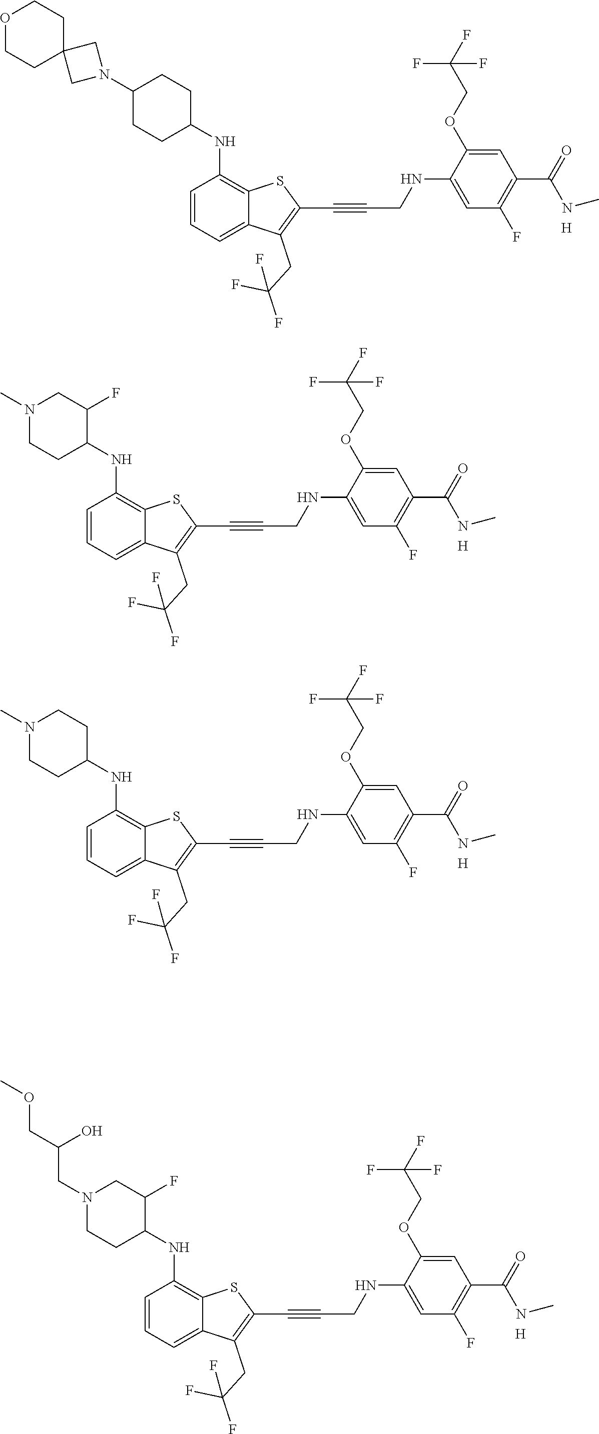 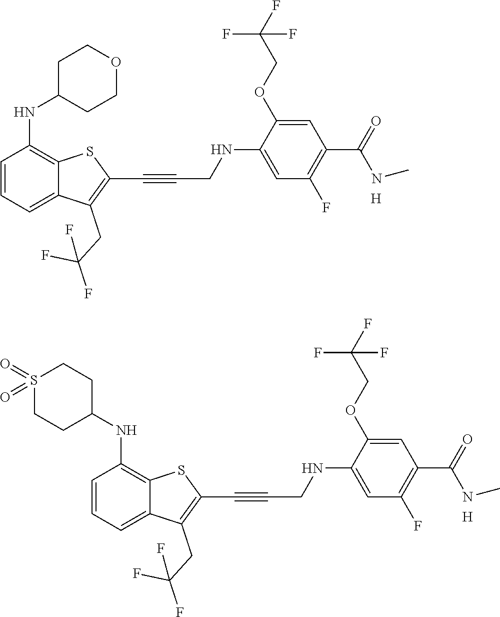 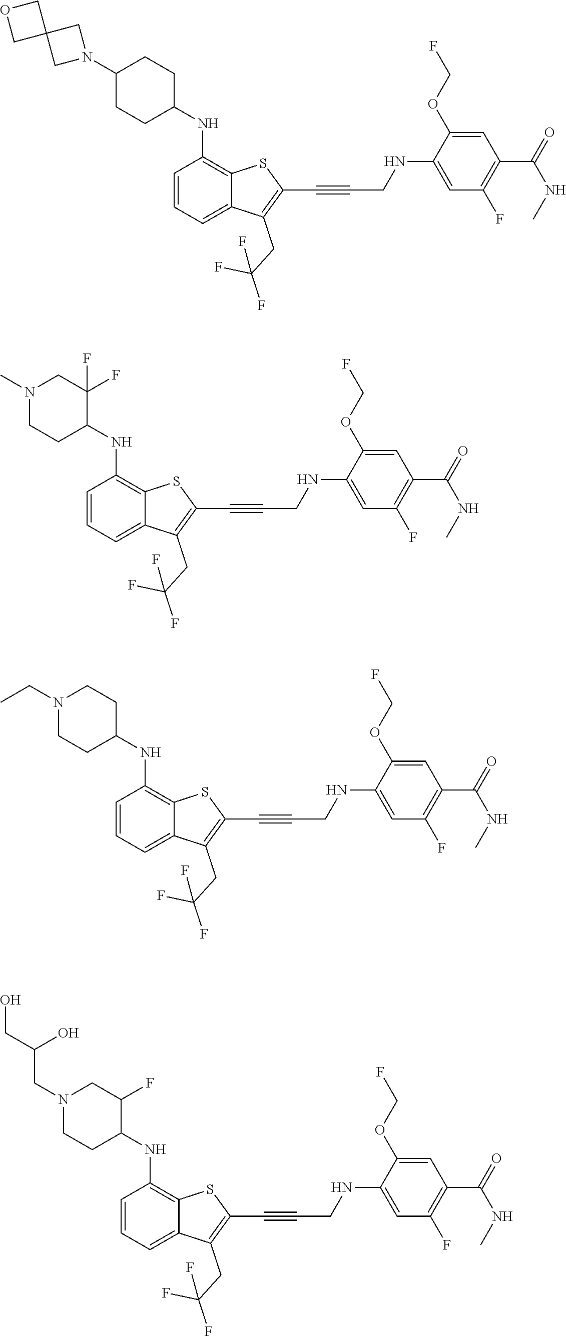 [SMILES: CNC(=O)c1cc(OCF)c(NCC#Cc2sc3c(NC4CCC(N5CC6(COC6)C5)CC4)cccc3c2CC(F)(F)F)cc1F ; CNC(=O)c1cc(OCF)c(NCC#Cc2sc3c(NC4CCN(C)CC4(F)F)cccc3c2CC(F)(F)F)cc1F ; CCN1CCC(Nc2cccc3c(CC(F)(F)F)c(C#CCNc4cc(F)c(C(=O)NC)cc4OCF)sc23)CC1 ; CNC(=O)c1cc(OCF)c(NCC#Cc2sc3c(NC4CCN(CC(O)CO)CC4F)cccc3c2CC(F)(F)F)cc1F] 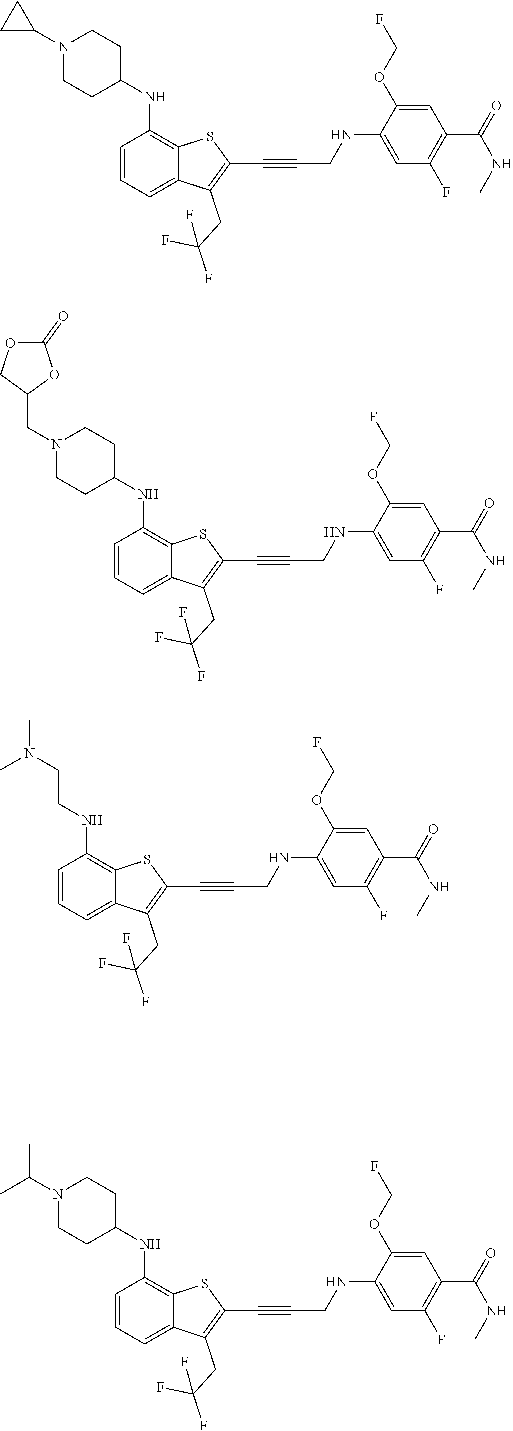 [SMILES: CNC(=O)c1cc(OCF)c(NCC#Cc2sc3c(NC4CCN(C5CC5)CC4)cccc3c2CC(F)(F)F)cc1F ; CNC(=O)c1cc(OCF)c(NCC#Cc2sc3c(NC4CCN(CC5COC(=O)O5)CC4)cccc3c2CC(F)(F)F)cc1F ; CNC(=O)c1cc(OCF)c(NCC#Cc2sc3c(NCCN(C)C)cccc3c2CC(F)(F)F)cc1F ; CNC(=O)c1cc(OCF)c(NCC#Cc2sc3c(NC4CCN(C(C)C)CC4)cccc3c2CC(F)(F)F)cc1F] 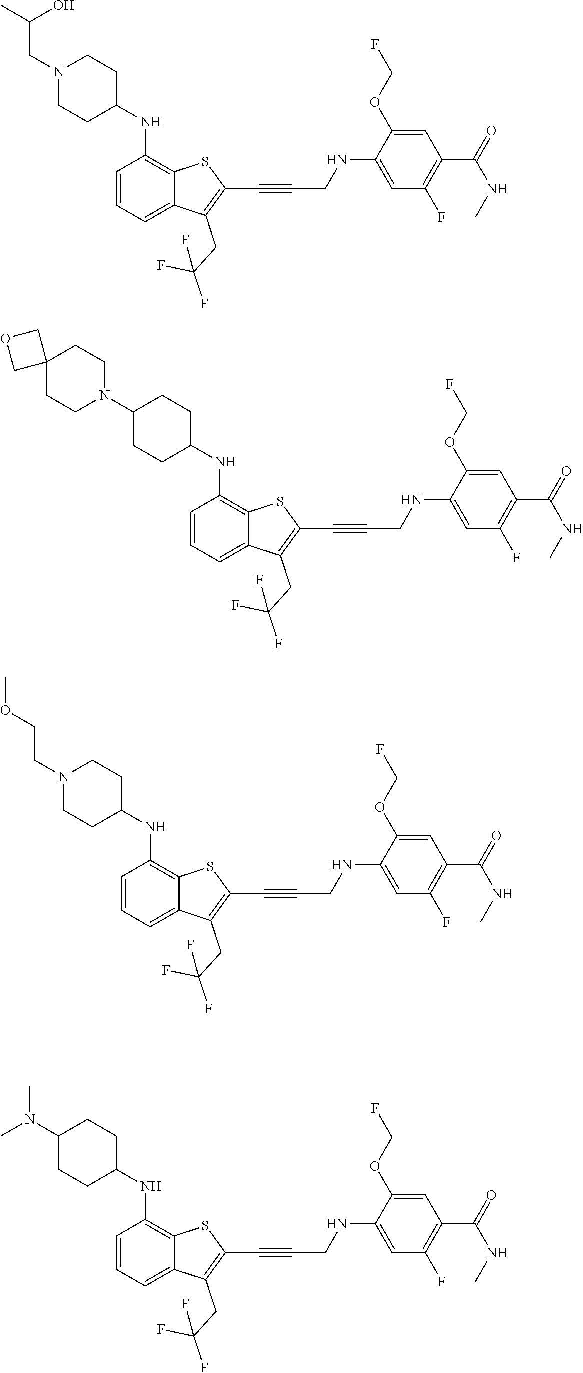 [SMILES: CNC(=O)c1cc(OCF)c(NCC#Cc2sc3c(NC4CCN(CC(C)O)CC4)cccc3c2CC(F)(F)F)cc1F ; CNC(=O)c1cc(OCF)c(NCC#Cc2sc3c(NC4CCC(N5CCC6(CC5)COC6)CC4)cccc3c2CC(F)(F)F)cc1F ; CNC(=O)c1cc(OCF)c(NCC#Cc2sc3c(NC4CCN(CCOC)CC4)cccc3c2CC(F)(F)F)cc1F ; CNC(=O)c1cc(OCF)c(NCC#Cc2sc3c(NC4CCC(N(C)C)CC4)cccc3c2CC(F)(F)F)cc1F] 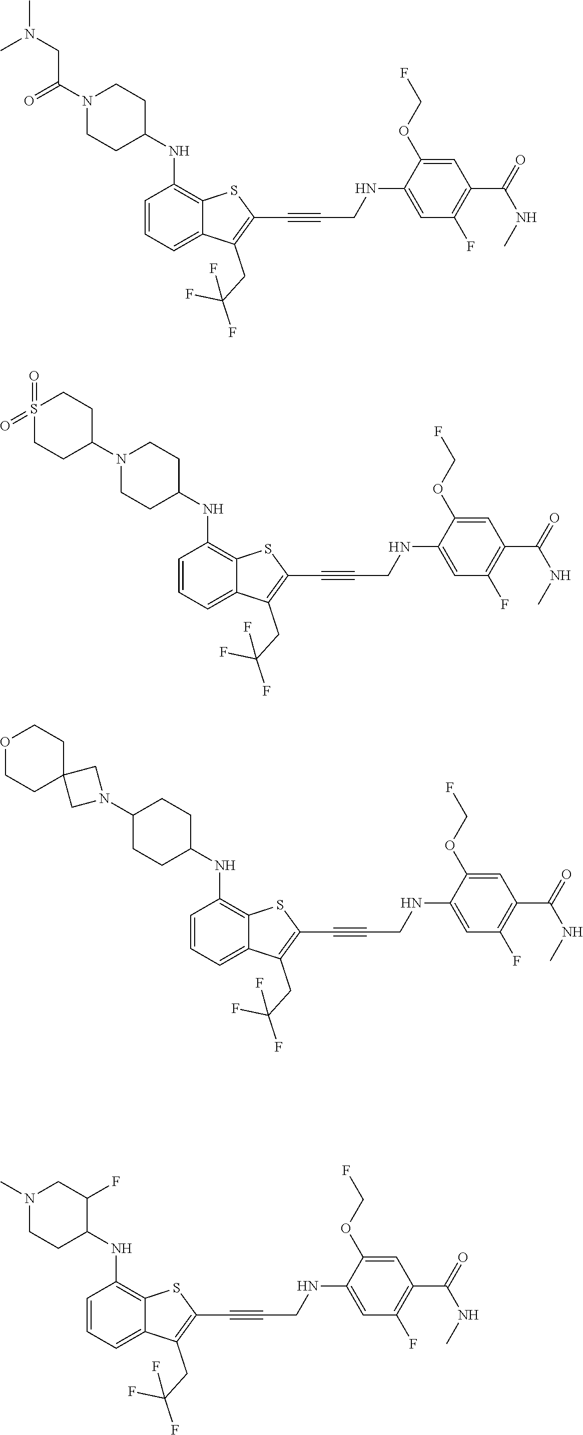 [SMILES: CNC(=O)c1cc(OCF)c(NCC#Cc2sc3c(NC4CCN(C(=O)CN(C)C)CC4)cccc3c2CC(F)(F)F)cc1F ; CNC(=O)c1cc(OCF)c(NCC#Cc2sc3c(NC4CCN(C5CCS(=O)(=O)CC5)CC4)cccc3c2CC(F)(F)F)cc1F ; CNC(=O)c1cc(OCF)c(NCC#Cc2sc3c(NC4CCC(N5CC6(CCOCC6)C5)CC4)cccc3c2CC(F)(F)F)cc1F ; CNC(=O)c1cc(OCF)c(NCC#Cc2sc3c(NC4CCN(C)CC4F)cccc3c2CC(F)(F)F)cc1F] 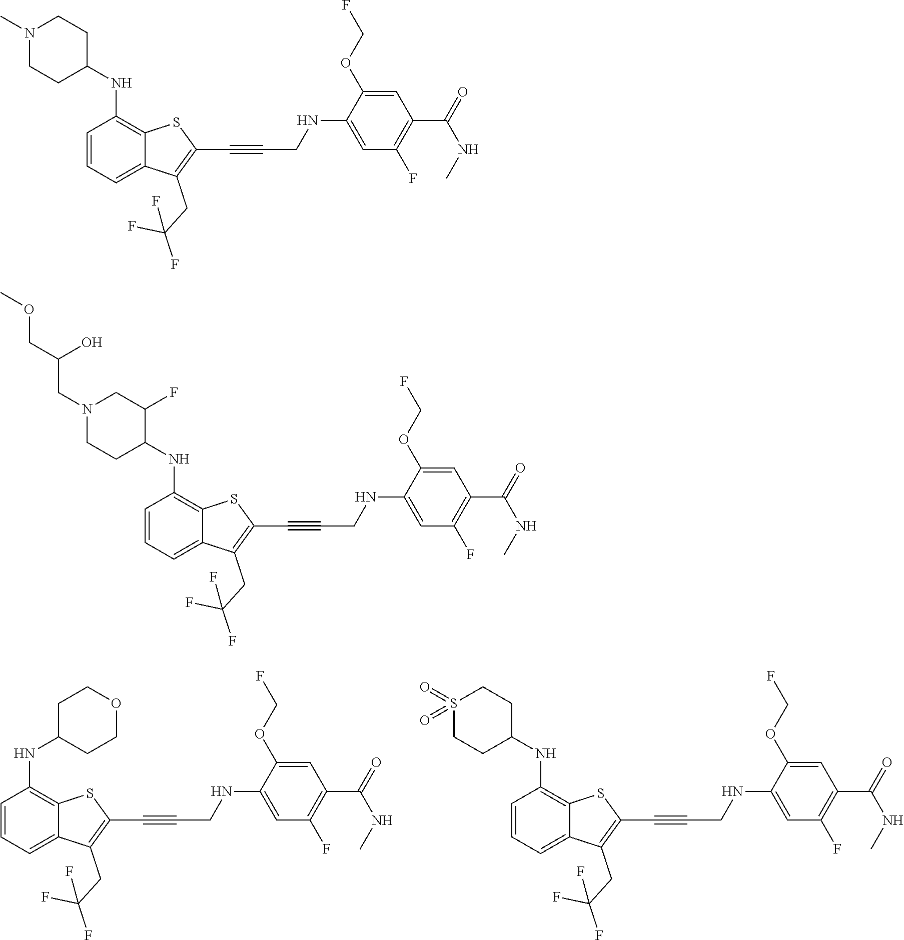 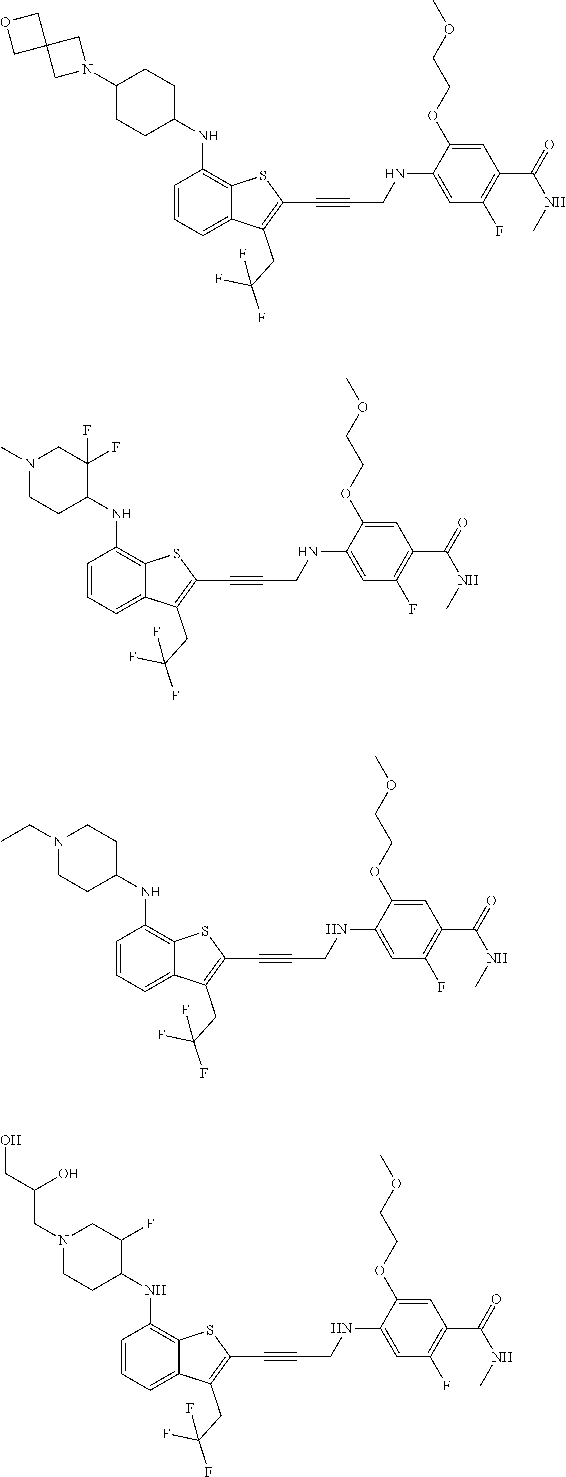 [SMILES: CNC(=O)c1cc(OCCOC)c(NCC#Cc2sc3c(NC4CCC(N5CC6(COC6)C5)CC4)cccc3c2CC(F)(F)F)cc1F ; CNC(=O)c1cc(OCCOC)c(NCC#Cc2sc3c(NC4CCN(C)CC4(F)F)cccc3c2CC(F)(F)F)cc1F ; CCN1CCC(Nc2cccc3c(CC(F)(F)F)c(C#CCNc4cc(F)c(C(=O)NC)cc4OCCOC)sc23)CC1 ; CNC(=O)c1cc(OCCOC)c(NCC#Cc2sc3c(NC4CCN(CC(O)CO)CC4F)cccc3c2CC(F)(F)F)cc1F] 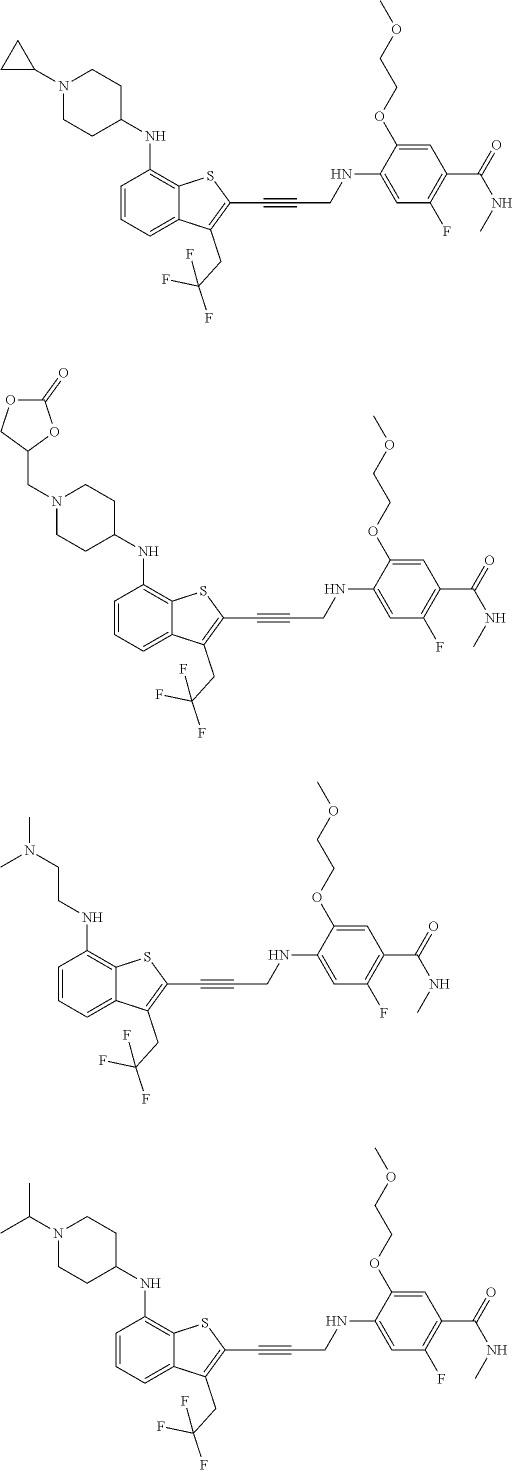 [SMILES: CNC(=O)c1cc(OCCOC)c(NCC#Cc2sc3c(NC4CCN(C5CC5)CC4)cccc3c2CC(F)(F)F)cc1F ; CNC(=O)c1cc(OCCOC)c(NCC#Cc2sc3c(NC4CCN(CC5COC(=O)O5)CC4)cccc3c2CC(F)(F)F)cc1F ; CNC(=O)c1cc(OCCOC)c(NCC#Cc2sc3c(NCCN(C)C)cccc3c2CC(F)(F)F)cc1F ; CNC(=O)c1cc(OCCOC)c(NCC#Cc2sc3c(NC4CCN(C(C)C)CC4)cccc3c2CC(F)(F)F)cc1F] 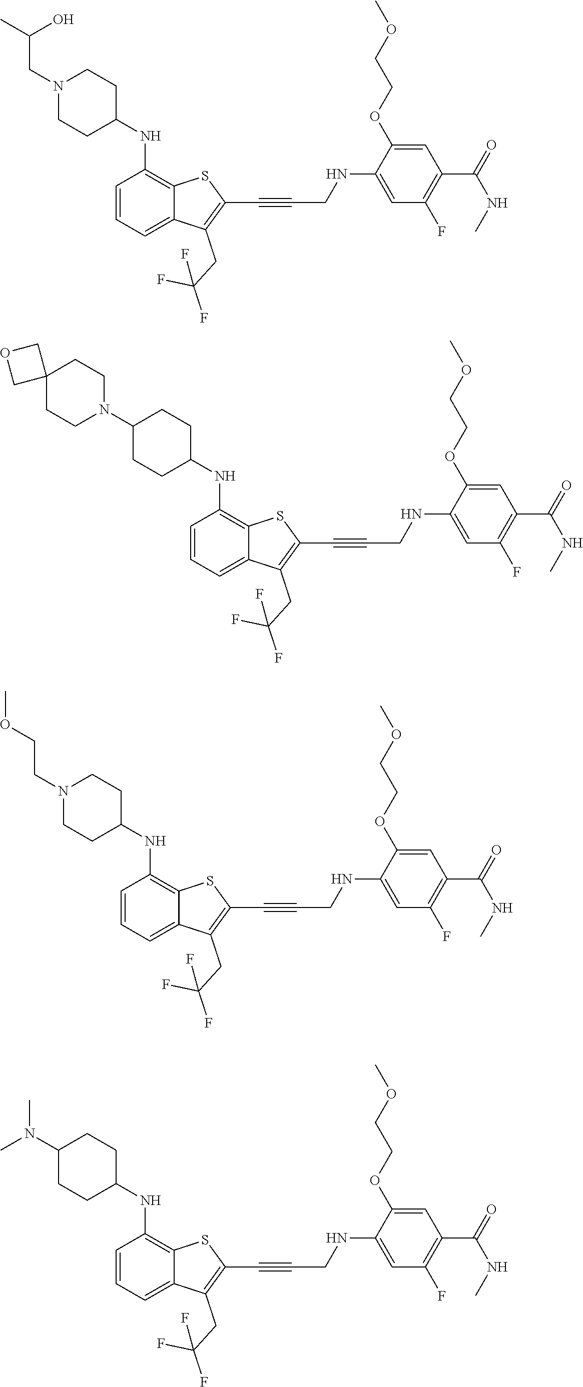 [SMILES: CNC(=O)c1cc(OCCOC)c(NCC#Cc2sc3c(NC4CCN(CC(C)O)CC4)cccc3c2CC(F)(F)F)cc1F ; CNC(=O)c1cc(OCCOC)c(NCC#Cc2sc3c(NC4CCC(N5CCC6(CC5)COC6)CC4)cccc3c2CC(F)(F)F)cc1F ; CNC(=O)c1cc(OCCOC)c(NCC#Cc2sc3c(NC4CCN(CCOC)CC4)cccc3c2CC(F)(F)F)cc1F ; CNC(=O)c1cc(OCCOC)c(NCC#Cc2sc3c(NC4CCC(N(C)C)CC4)cccc3c2CC(F)(F)F)cc1F] 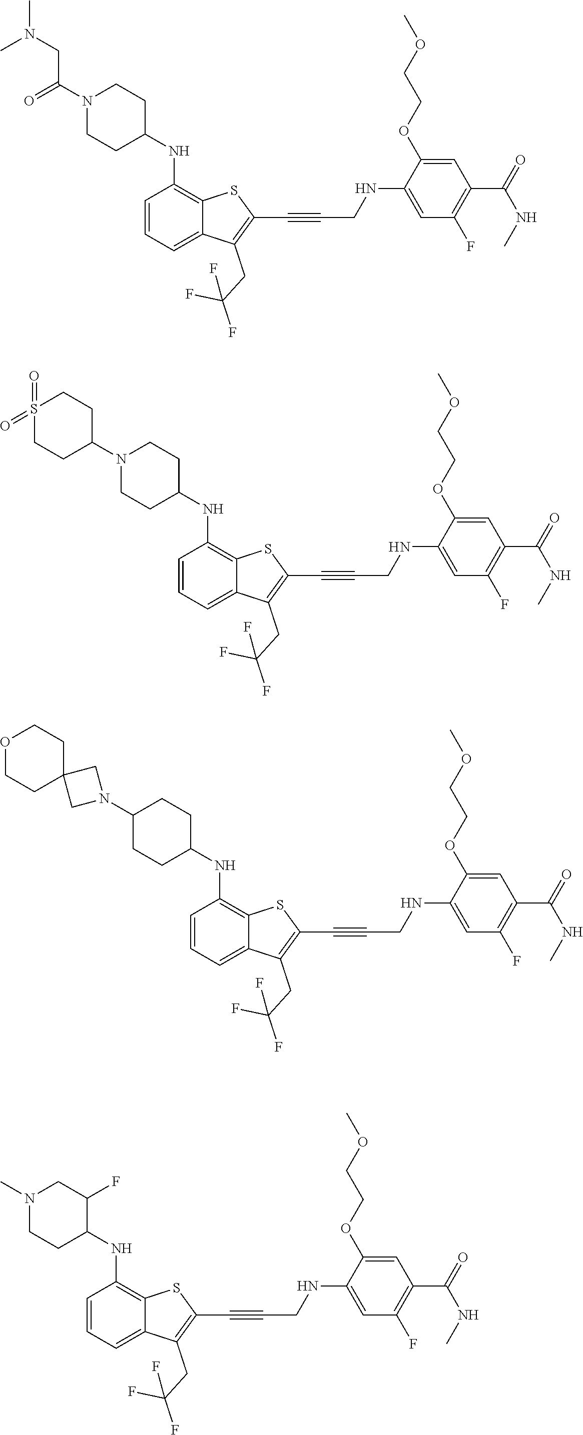 [SMILES: CNC(=O)c1cc(OCCOC)c(NCC#Cc2sc3c(NC4CCN(C(=O)CN(C)C)CC4)cccc3c2CC(F)(F)F)cc1F ; CNC(=O)c1cc(OCCOC)c(NCC#Cc2sc3c(NC4CCN(C5CCS(=O)(=O)CC5)CC4)cccc3c2CC(F)(F)F)cc1F ; CNC(=O)c1cc(OCCOC)c(NCC#Cc2sc3c(NC4CCC(N5CC6(CCOCC6)C5)CC4)cccc3c2CC(F)(F)F)cc1F ; CNC(=O)c1cc(OCCOC)c(NCC#Cc2sc3c(NC4CCN(C)CC4F)cccc3c2CC(F)(F)F)cc1F] 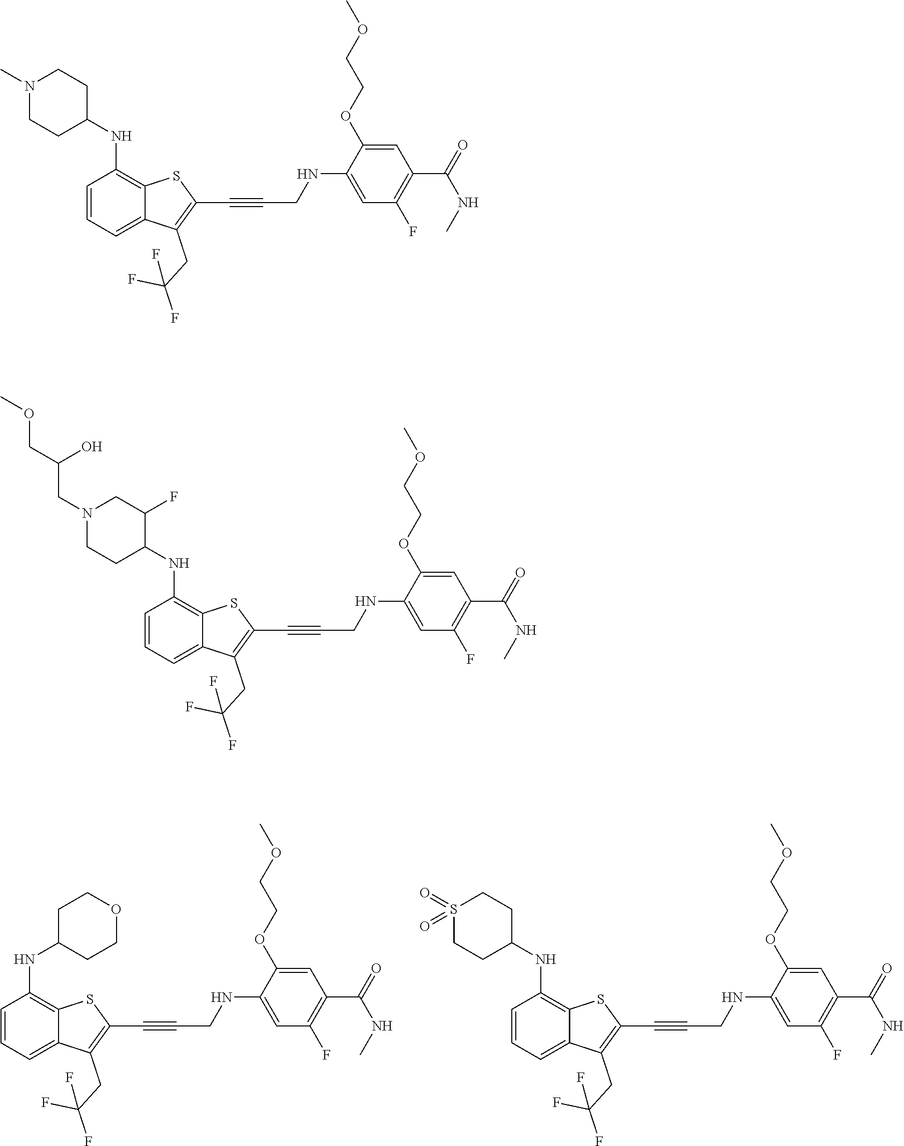 [SMILES: CNC(=O)c1cc(OCCOC)c(NCC#Cc2sc3c(NC4CCN(C)CC4)cccc3c2CC(F)(F)F)cc1F ; CNC(=O)c1cc(OCCOC)c(NCC#Cc2sc3c(NC4CCN(CC(O)COC)CC4F)cccc3c2CC(F)(F)F)cc1F ; CNC(=O)c1cc(OCCOC)c(NCC#Cc2sc3c(NC4CCOCC4)cccc3c2CC(F)(F)F)cc1F ; CNC(=O)c1cc(OCCOC)c(NCC#Cc2sc3c(NC4CCS(=O)(=O)CC4)cccc3c2CC(F)(F)F)cc1F] 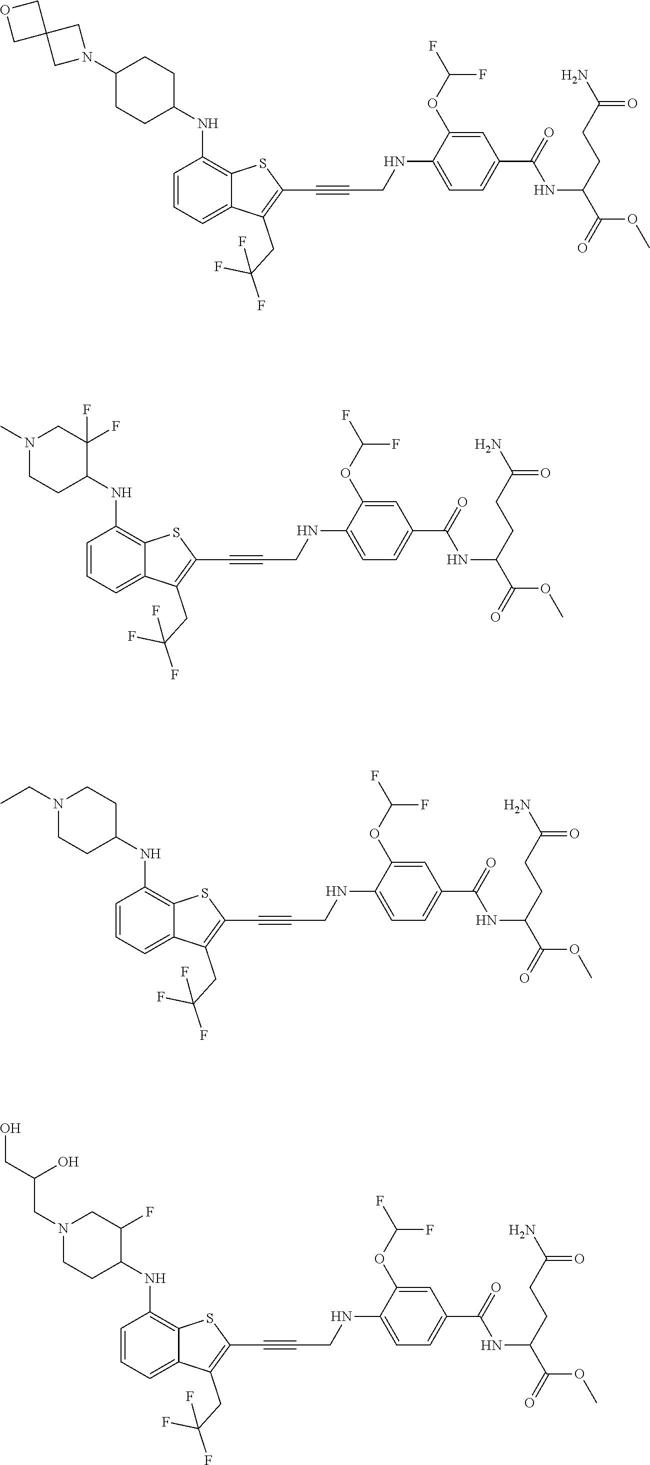 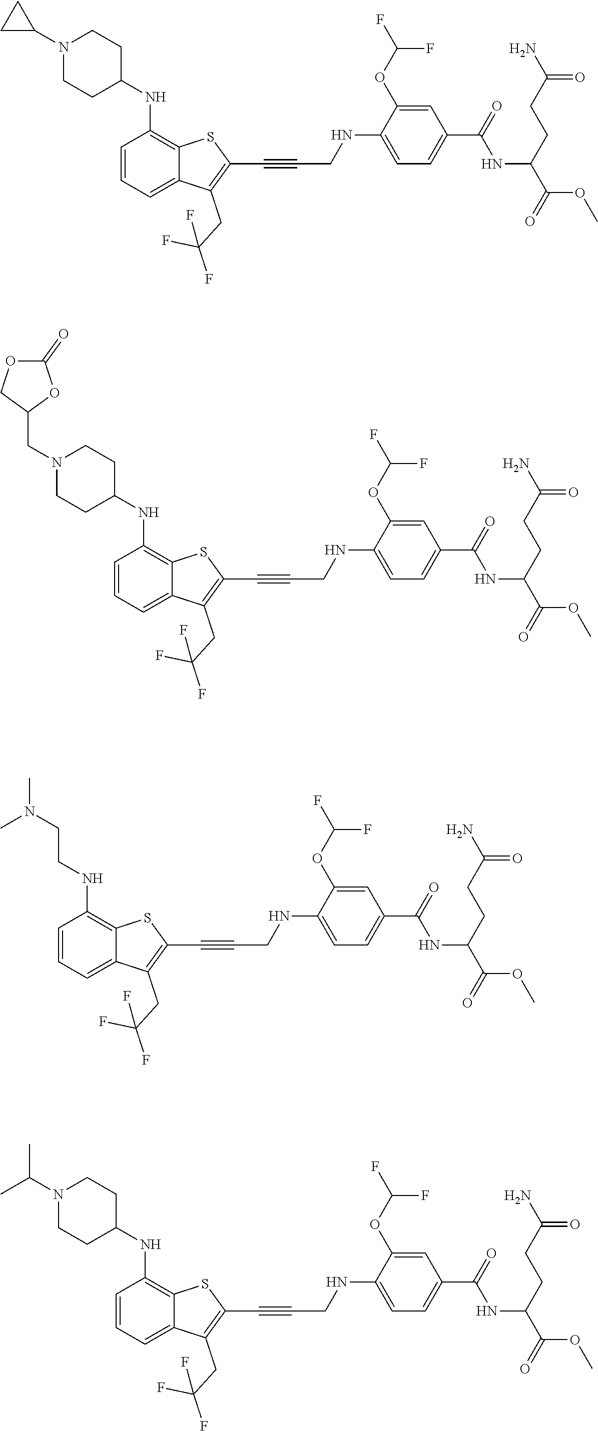 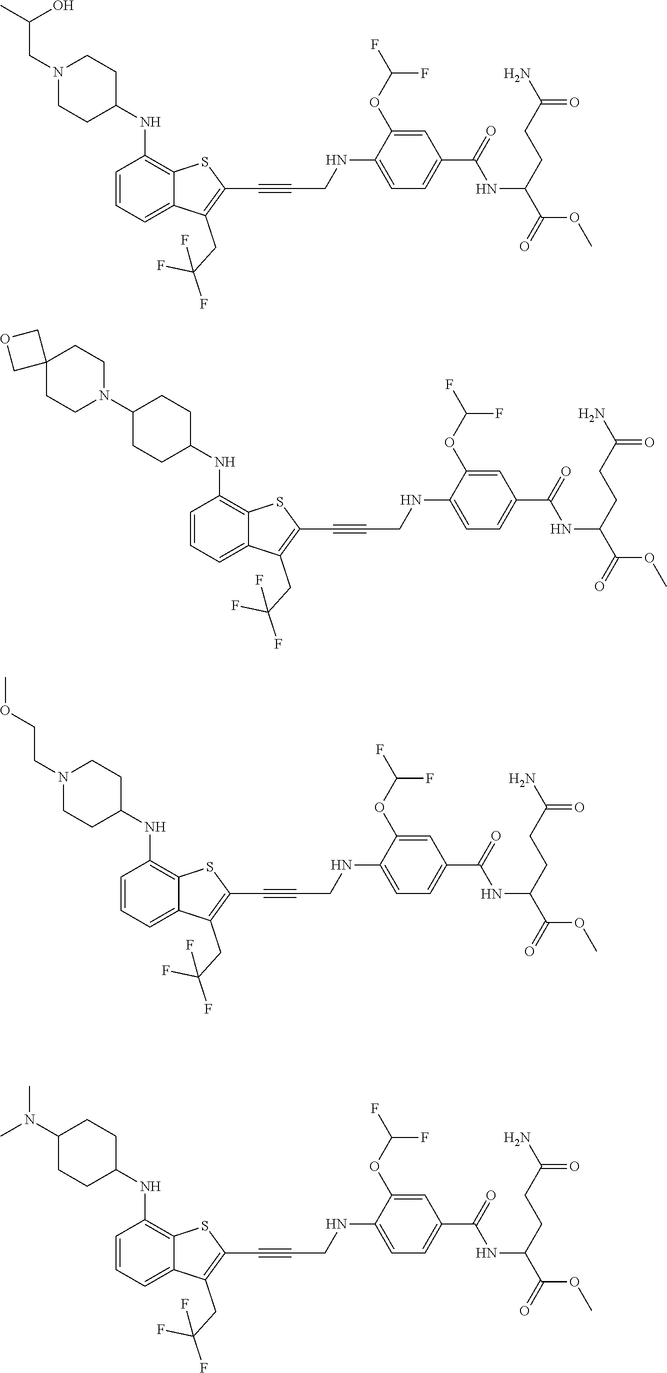 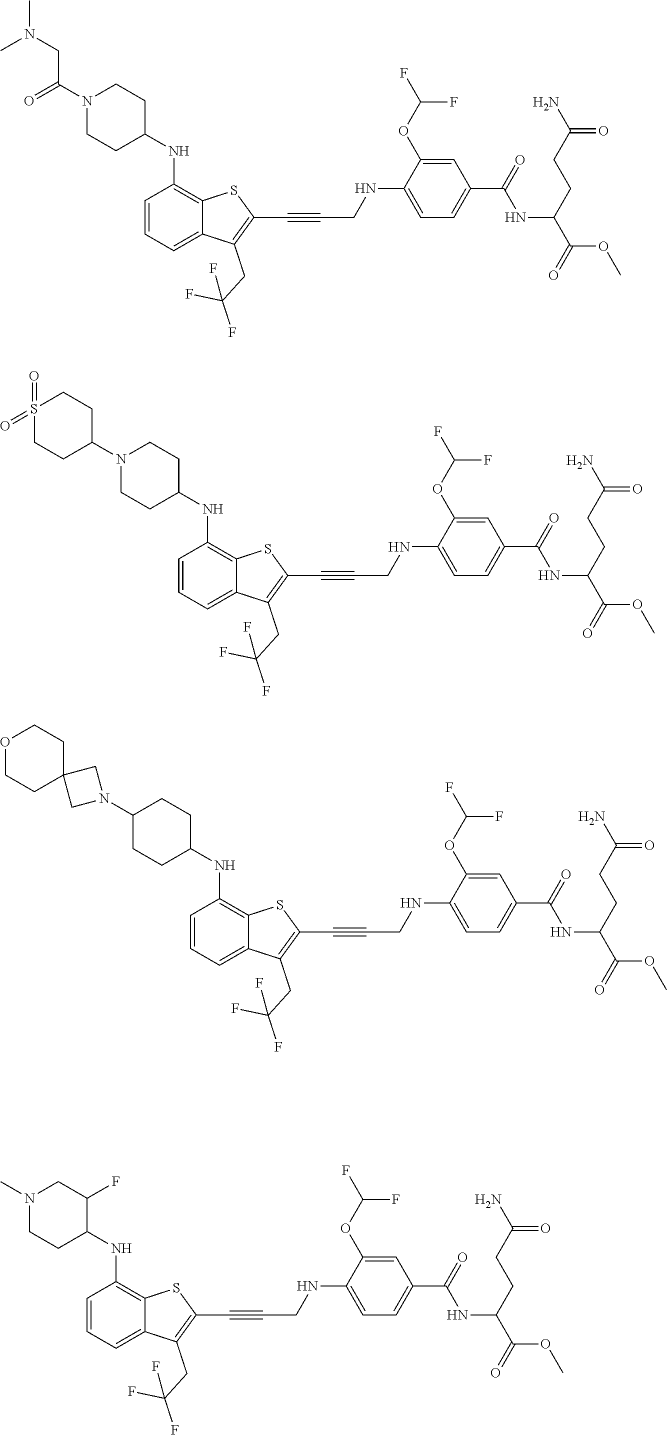 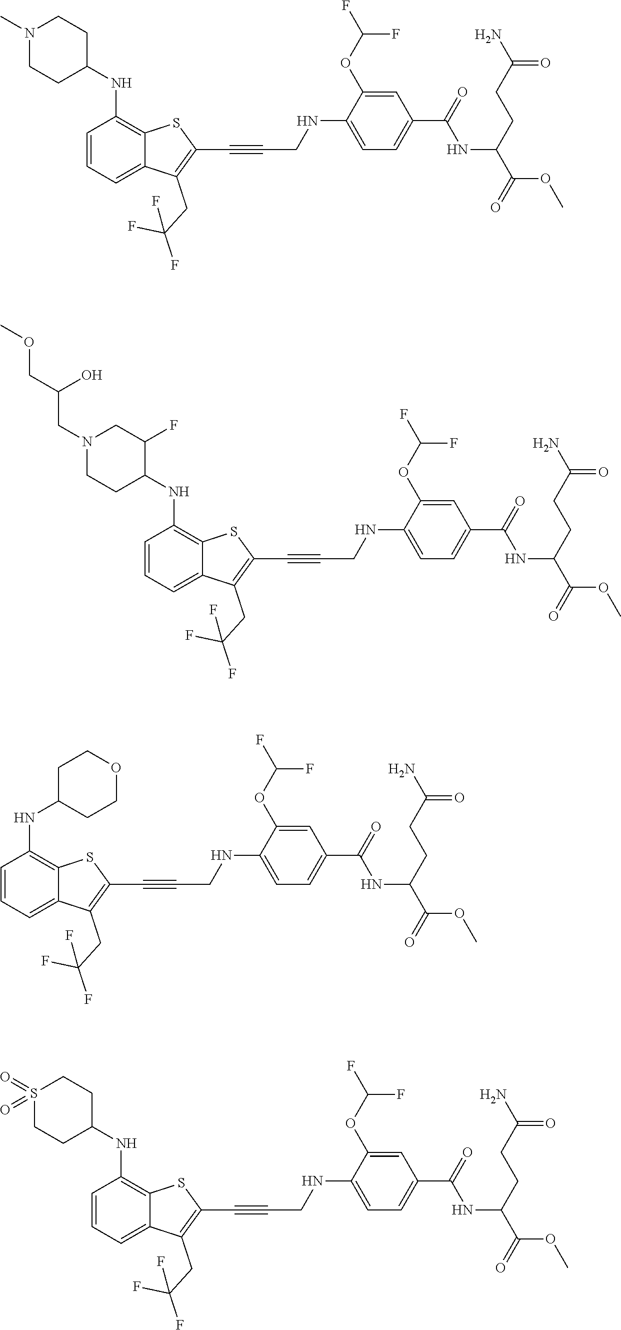 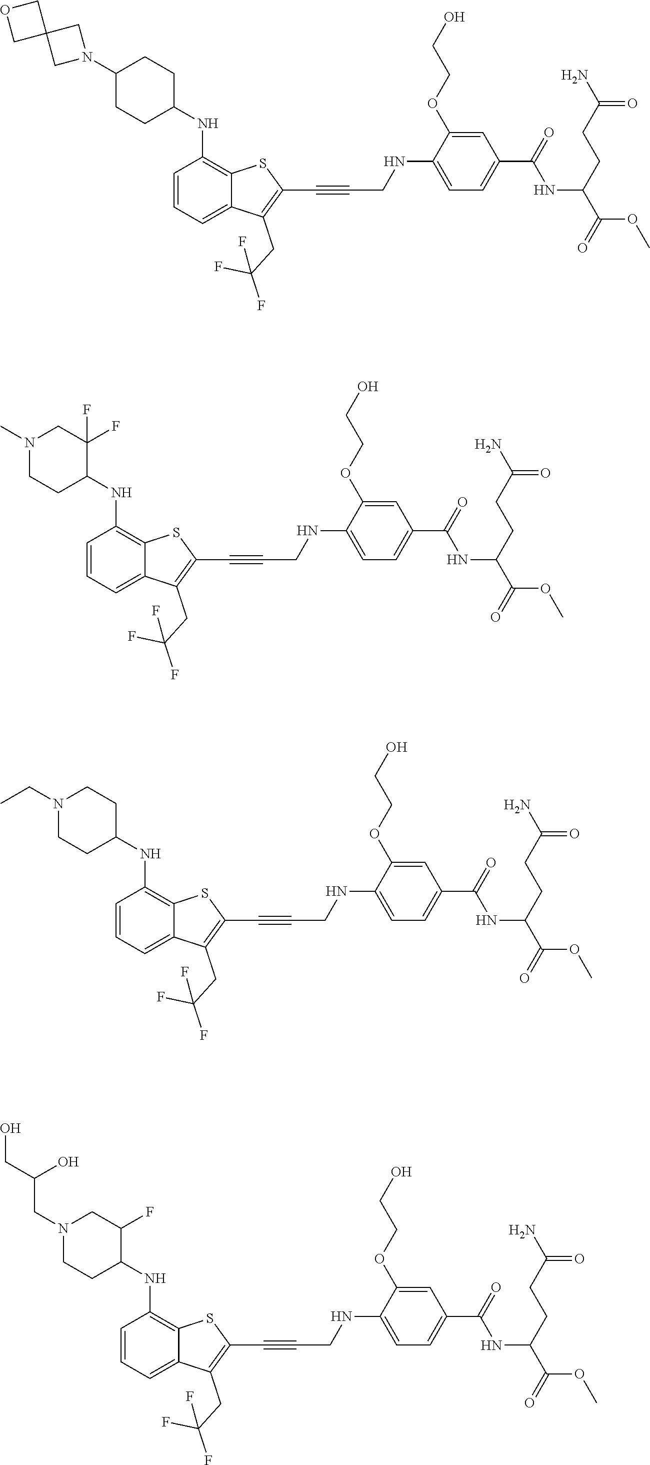 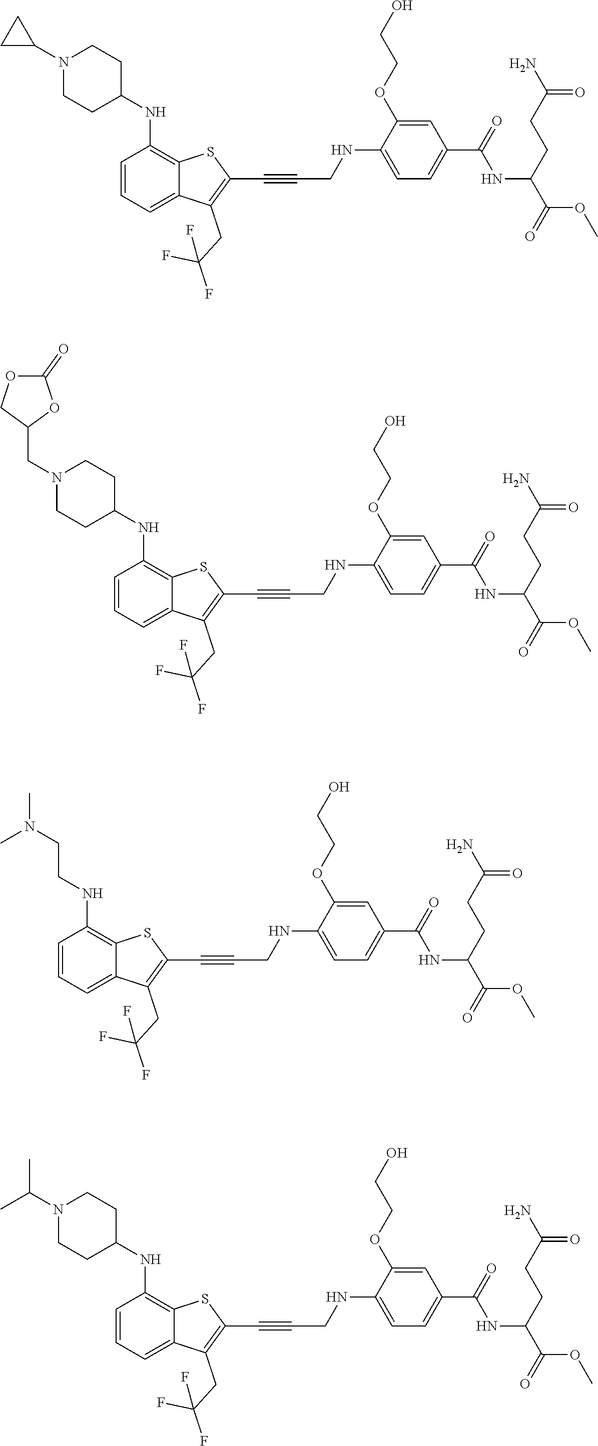 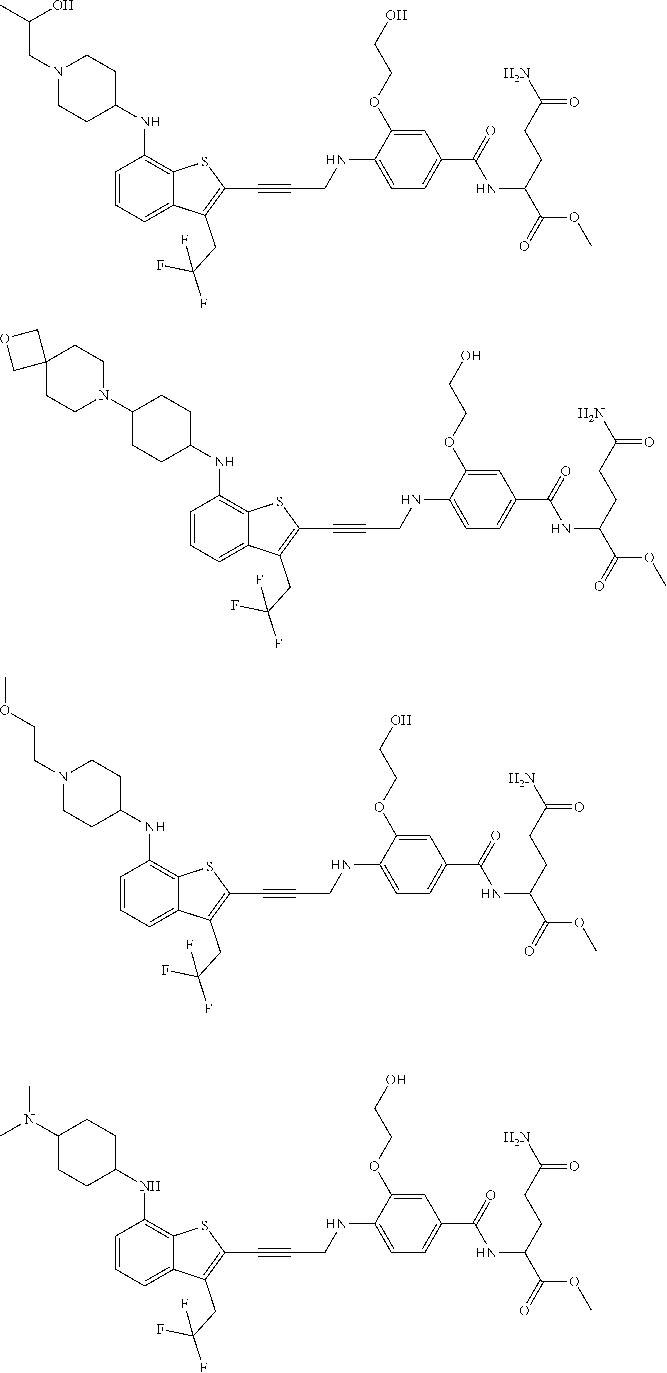 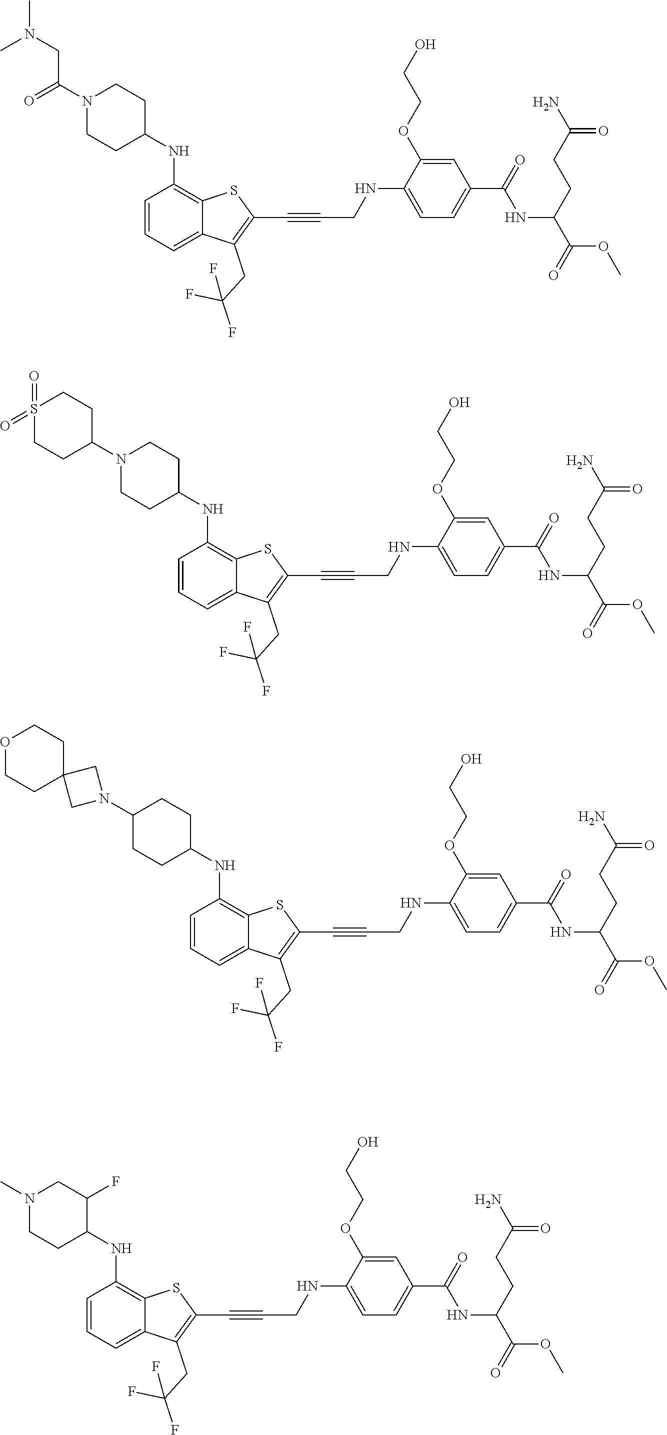 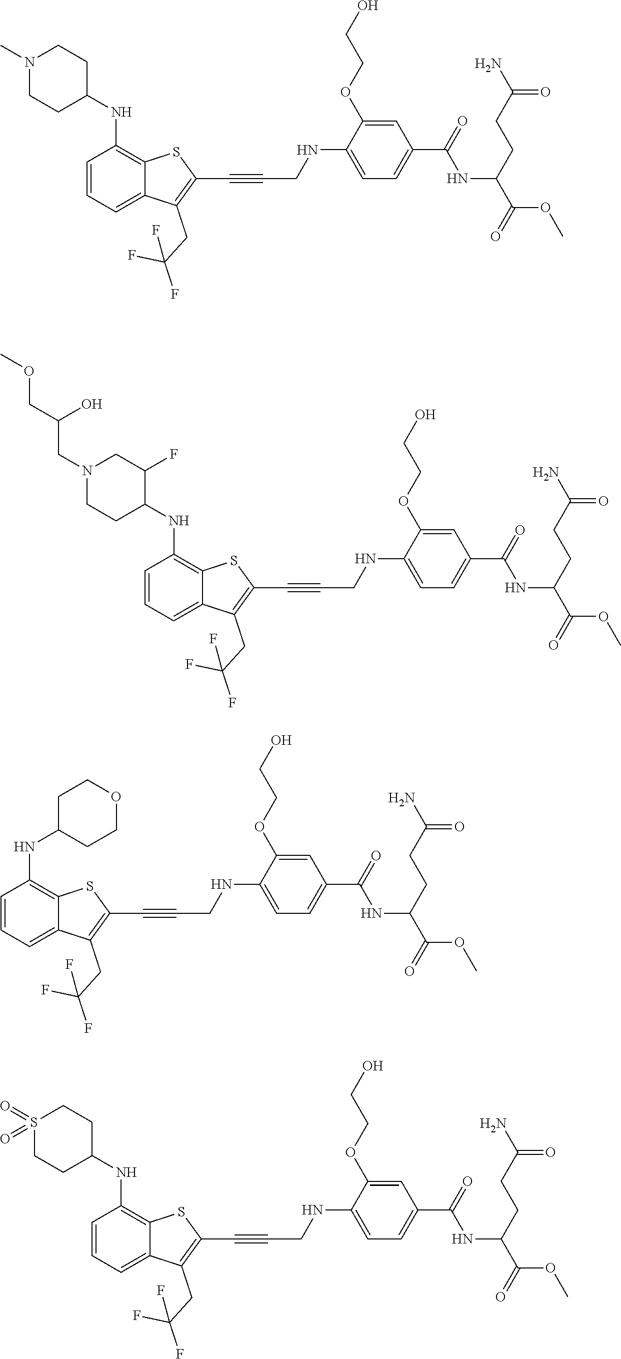 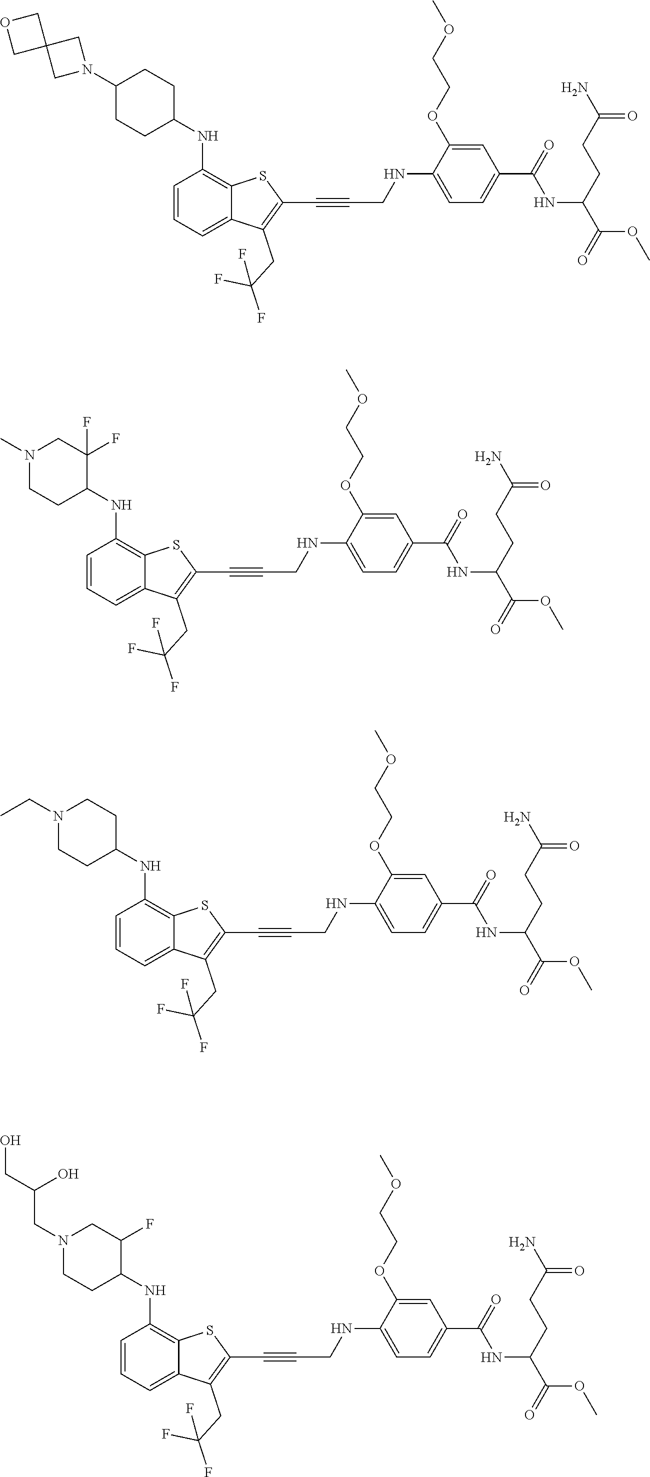 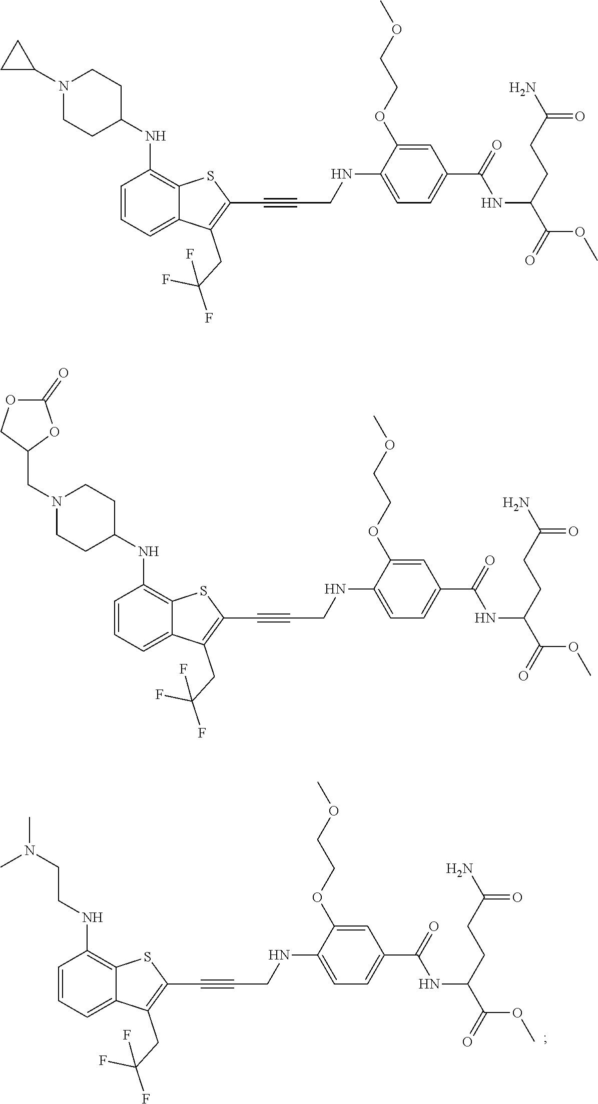
or the compound of formula (I) is selected from: 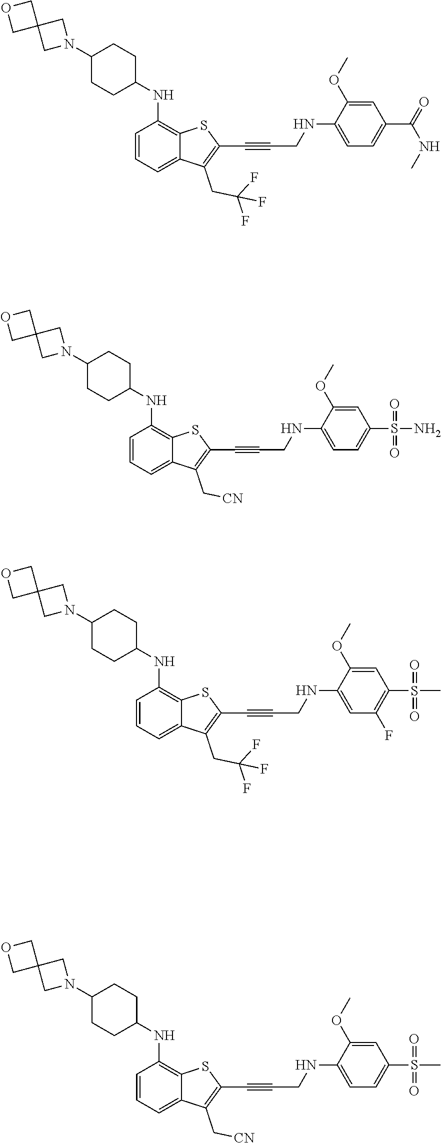 [SMILES: CNC(=O)c1ccc(NCC#Cc2sc3c(NC4CCC(N5CC6(COC6)C5)CC4)cccc3c2CC(F)(F)F)c(OC)c1 ; COc1cc(S(N)(=O)=O)ccc1NCC#Cc1sc2c(NC3CCC(N4CC5(COC5)C4)CC3)cccc2c1CC#N ; COc1cc(S(C)(=O)=O)c(F)cc1NCC#Cc1sc2c(NC3CCC(N4CC5(COC5)C4)CC3)cccc2c1CC(F)(F)F ; COc1cc(S(C)(=O)=O)ccc1NCC#Cc1sc2c(NC3CCC(N4CC5(COC5)C4)CC3)cccc2c1CC#N] 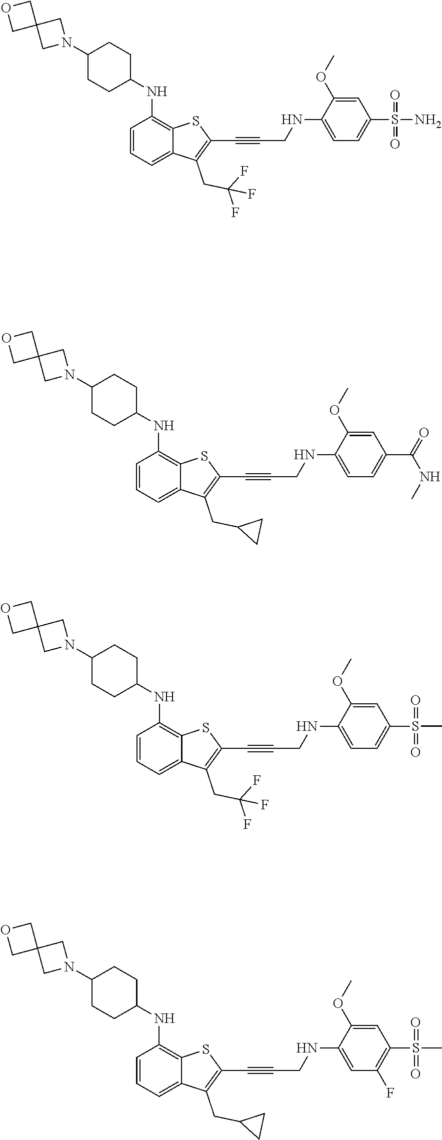 [SMILES: COc1cc(S(N)(=O)=O)ccc1NCC#Cc1sc2c(NC3CCC(N4CC5(COC5)C4)CC3)cccc2c1CC(F)(F)F ; CNC(=O)c1ccc(NCC#Cc2sc3c(NC4CCC(N5CC6(COC6)C5)CC4)cccc3c2CC2CC2)c(OC)c1 ; COc1cc(S(C)(=O)=O)ccc1NCC#Cc1sc2c(NC3CCC(N4CC5(COC5)C4)CC3)cccc2c1CC(F)(F)F ; COc1cc(S(C)(=O)=O)c(F)cc1NCC#Cc1sc2c(NC3CCC(N4CC5(COC5)C4)CC3)cccc2c1CC1CC1] 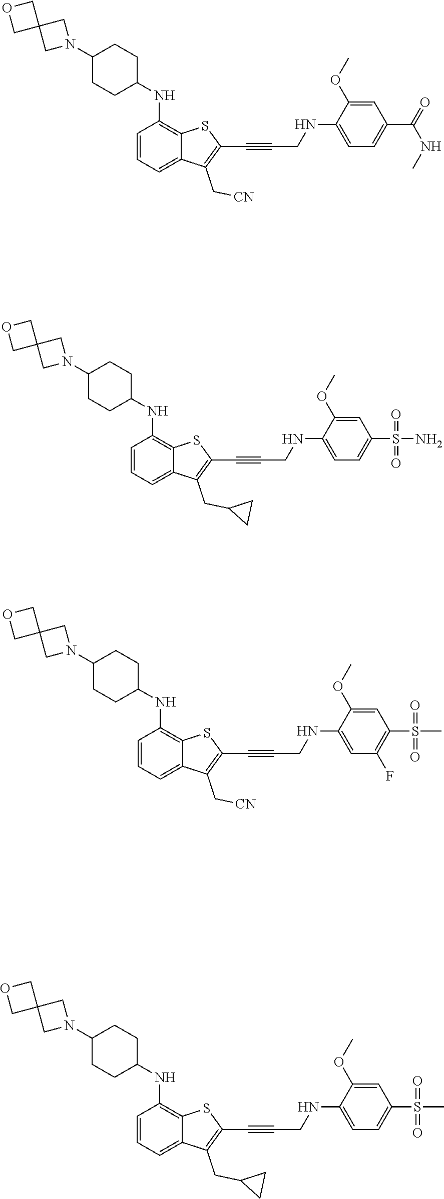 [SMILES: CNC(=O)c1ccc(NCC#Cc2sc3c(NC4CCC(N5CC6(COC6)C5)CC4)cccc3c2CC#N)c(OC)c1 ; COc1cc(S(N)(=O)=O)ccc1NCC#Cc1sc2c(NC3CCC(N4CC5(COC5)C4)CC3)cccc2c1CC1CC1 ; COc1cc(S(C)(=O)=O)c(F)cc1NCC#Cc1sc2c(NC3CCC(N4CC5(COC5)C4)CC3)cccc2c1CC#N ; COc1cc(S(C)(=O)=O)ccc1NCC#Cc1sc2c(NC3CCC(N4CC5(COC5)C4)CC3)cccc2c1CC1CC1] 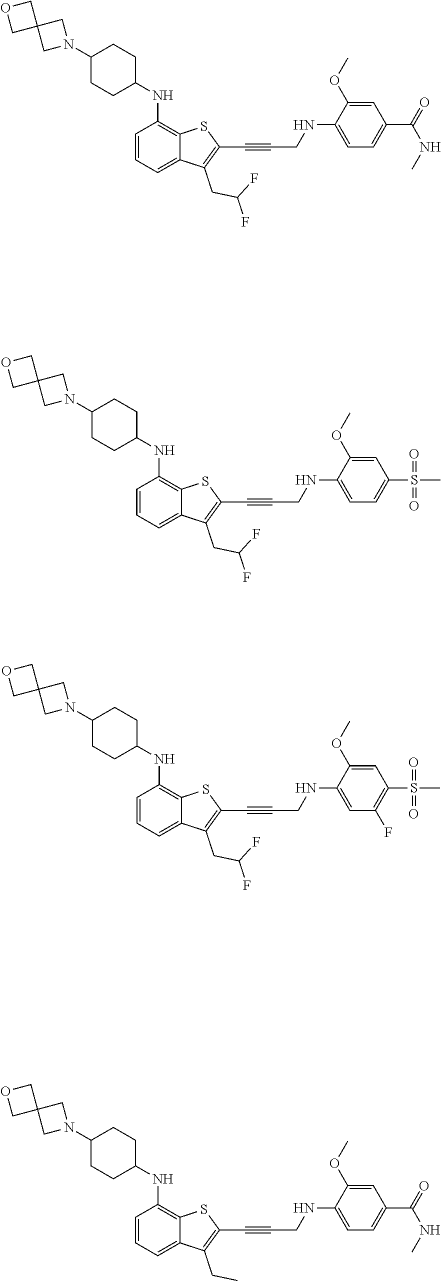 [SMILES: CNC(=O)c1ccc(NCC#Cc2sc3c(NC4CCC(N5CC6(COC6)C5)CC4)cccc3c2CC(F)F)c(OC)c1 ; COc1cc(S(C)(=O)=O)ccc1NCC#Cc1sc2c(NC3CCC(N4CC5(COC5)C4)CC3)cccc2c1CC(F)F ; COc1cc(S(C)(=O)=O)c(F)cc1NCC#Cc1sc2c(NC3CCC(N4CC5(COC5)C4)CC3)cccc2c1CC(F)F ; CCc1c(C#CCNc2ccc(C(=O)NC)cc2OC)sc2c(NC3CCC(N4CC5(COC5)C4)CC3)cccc12] 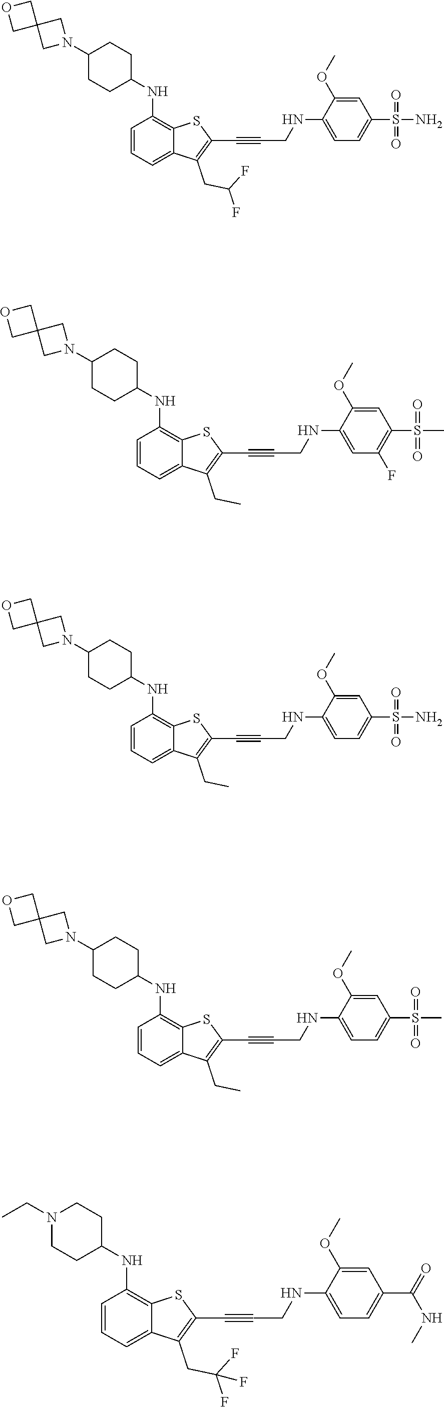 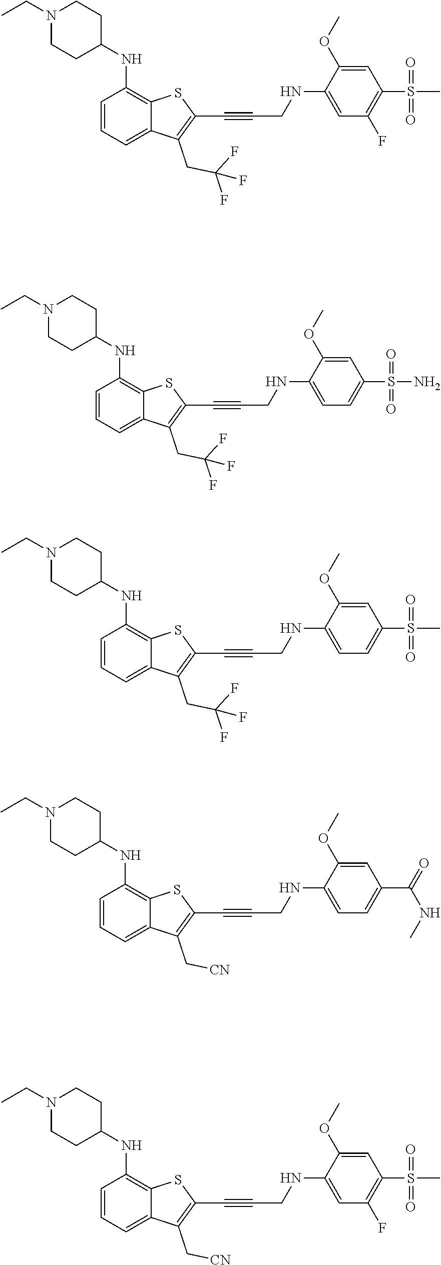 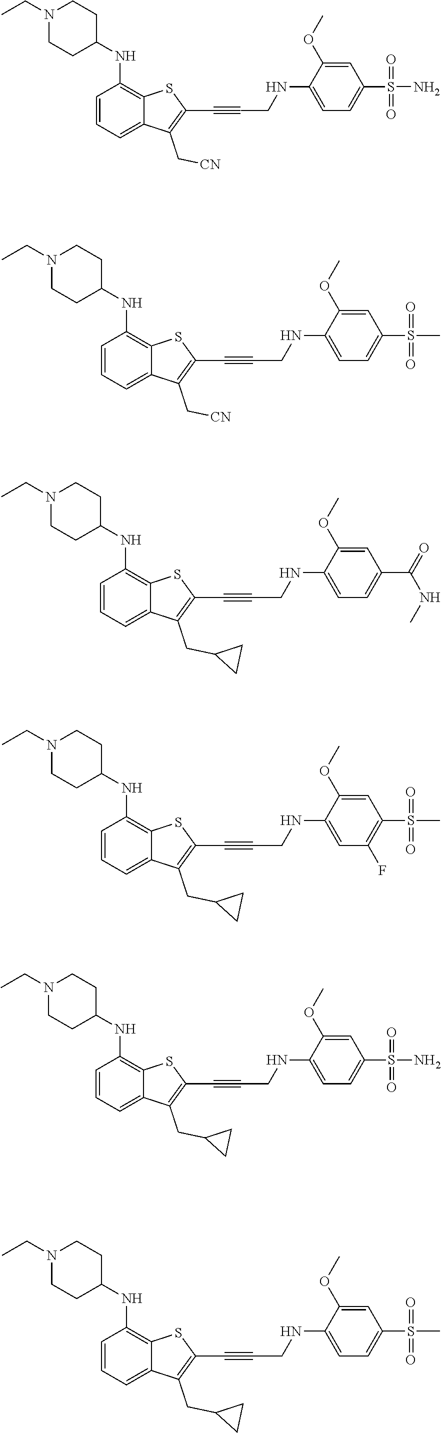 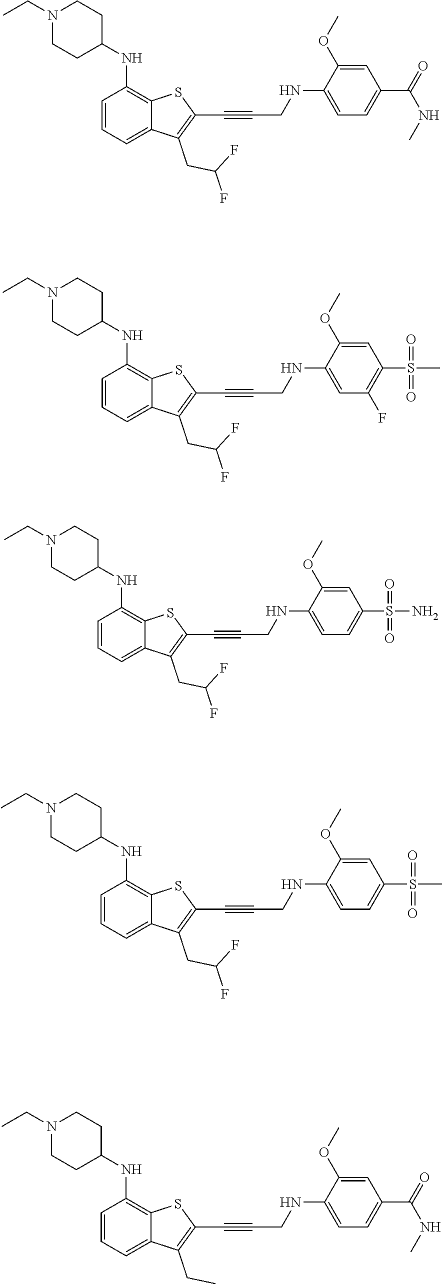 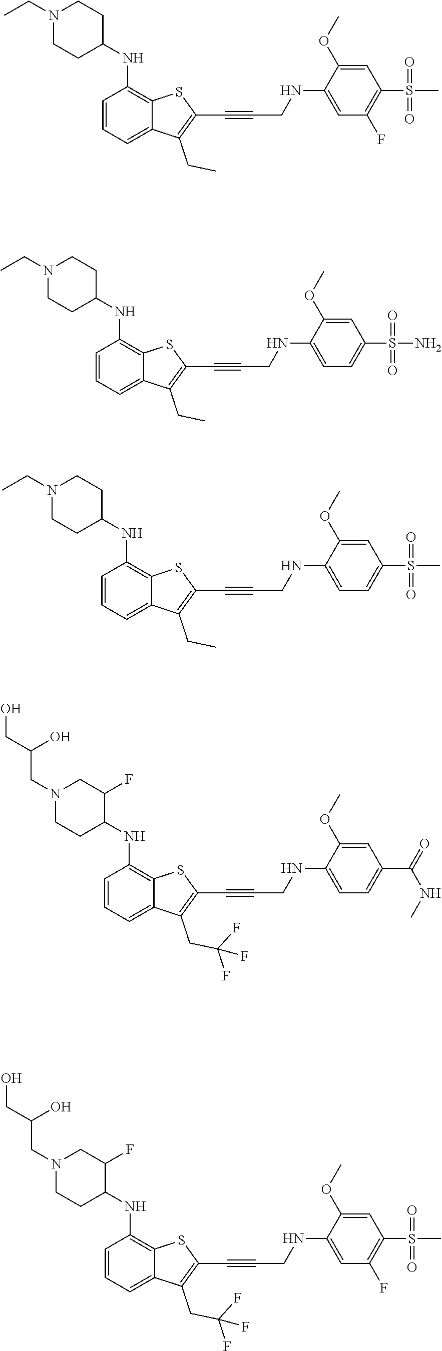 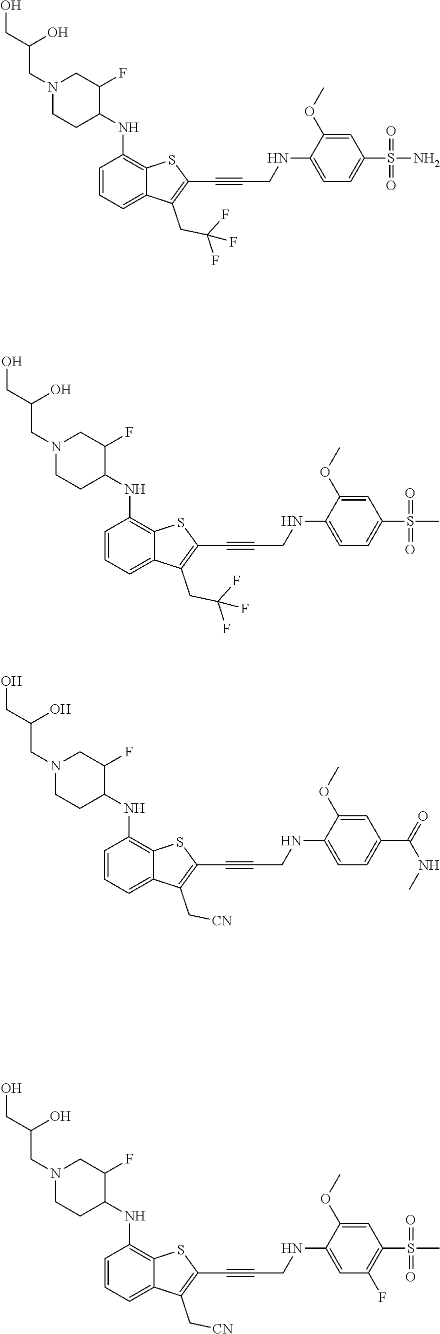 [SMILES: COc1cc(S(N)(=O)=O)ccc1NCC#Cc1sc2c(NC3CCN(CC(O)CO)CC3F)cccc2c1CC(F)(F)F ; COc1cc(S(C)(=O)=O)ccc1NCC#Cc1sc2c(NC3CCN(CC(O)CO)CC3F)cccc2c1CC(F)(F)F ; CNC(=O)c1ccc(NCC#Cc2sc3c(NC4CCN(CC(O)CO)CC4F)cccc3c2CC#N)c(OC)c1 ; COc1cc(S(C)(=O)=O)c(F)cc1NCC#Cc1sc2c(NC3CCN(CC(O)CO)CC3F)cccc2c1CC#N] 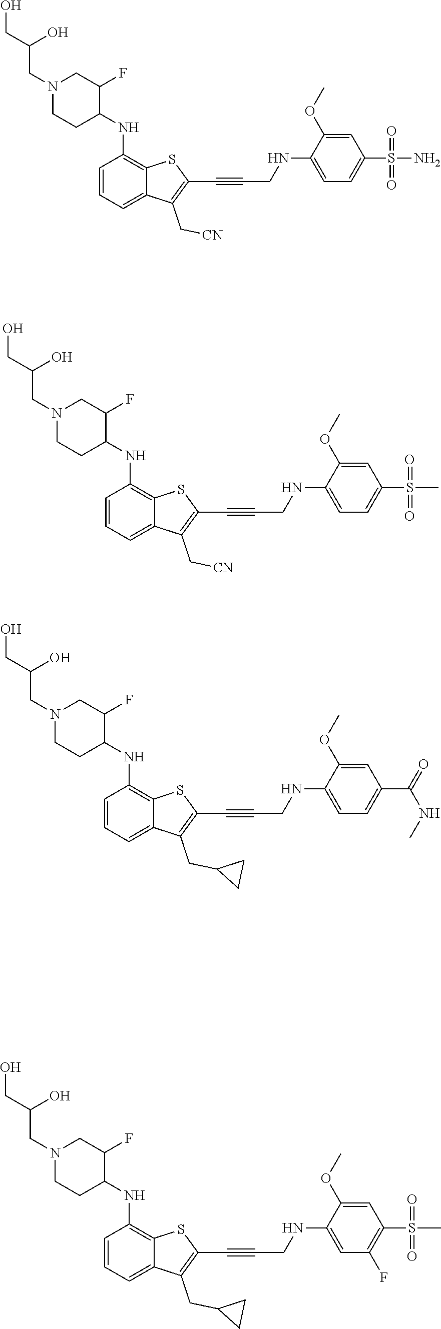 [SMILES: COc1cc(S(N)(=O)=O)ccc1NCC#Cc1sc2c(NC3CCN(CC(O)CO)CC3F)cccc2c1CC#N ; COc1cc(S(C)(=O)=O)ccc1NCC#Cc1sc2c(NC3CCN(CC(O)CO)CC3F)cccc2c1CC#N ; CNC(=O)c1ccc(NCC#Cc2sc3c(NC4CCN(CC(O)CO)CC4F)cccc3c2CC2CC2)c(OC)c1 ; COc1cc(S(C)(=O)=O)c(F)cc1NCC#Cc1sc2c(NC3CCN(CC(O)CO)CC3F)cccc2c1CC1CC1] 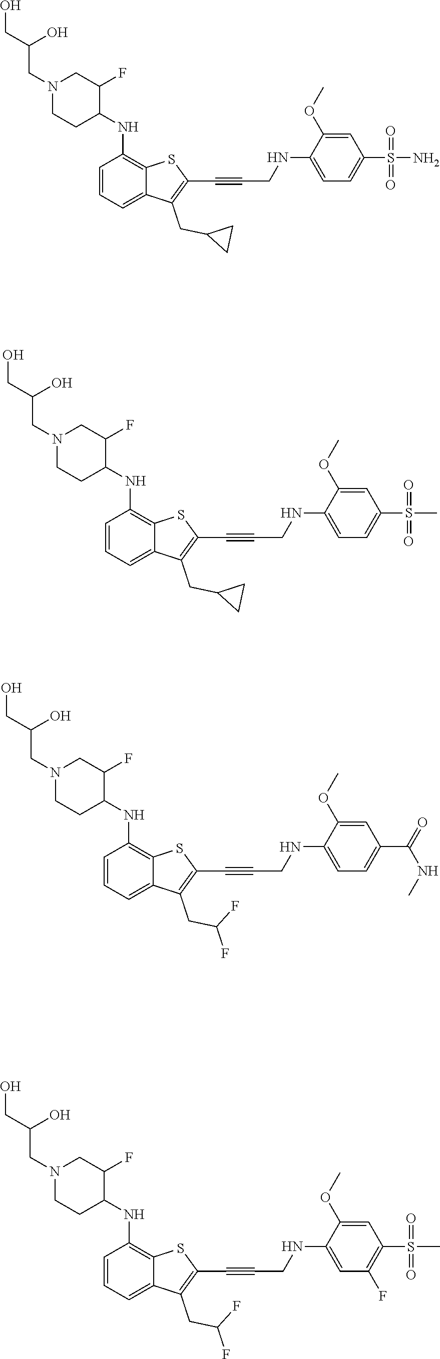 [SMILES: COc1cc(S(N)(=O)=O)ccc1NCC#Cc1sc2c(NC3CCN(CC(O)CO)CC3F)cccc2c1CC1CC1 ; COc1cc(S(C)(=O)=O)ccc1NCC#Cc1sc2c(NC3CCN(CC(O)CO)CC3F)cccc2c1CC1CC1 ; CNC(=O)c1ccc(NCC#Cc2sc3c(NC4CCN(CC(O)CO)CC4F)cccc3c2CC(F)F)c(OC)c1 ; COc1cc(S(C)(=O)=O)c(F)cc1NCC#Cc1sc2c(NC3CCN(CC(O)CO)CC3F)cccc2c1CC(F)F] 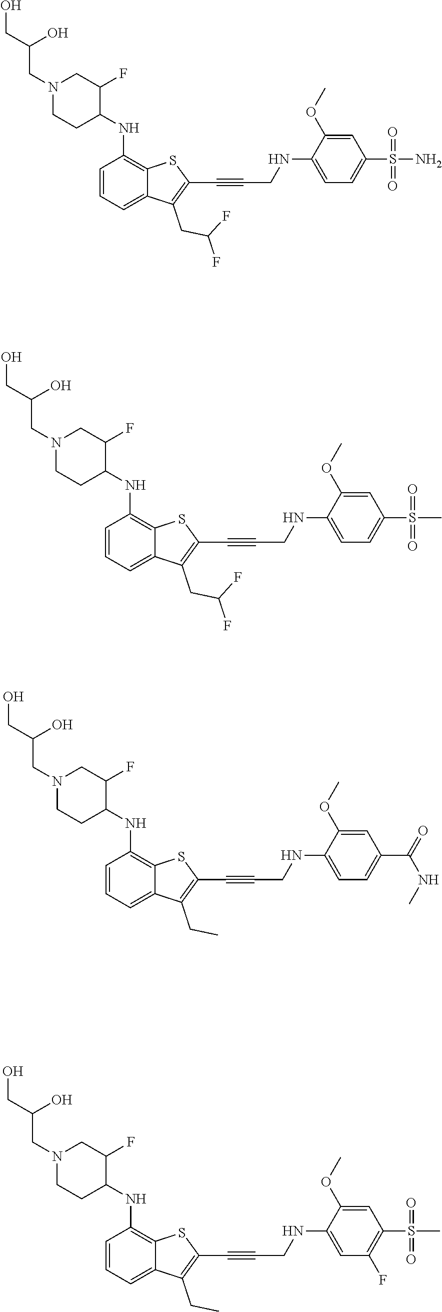 [SMILES: COc1cc(S(N)(=O)=O)ccc1NCC#Cc1sc2c(NC3CCN(CC(O)CO)CC3F)cccc2c1CC(F)F ; COc1cc(S(C)(=O)=O)ccc1NCC#Cc1sc2c(NC3CCN(CC(O)CO)CC3F)cccc2c1CC(F)F ; CCc1c(C#CCNc2ccc(C(=O)NC)cc2OC)sc2c(NC3CCN(CC(O)CO)CC3F)cccc12 ; CCc1c(C#CCNc2cc(F)c(S(C)(=O)=O)cc2OC)sc2c(NC3CCN(CC(O)CO)CC3F)cccc12] 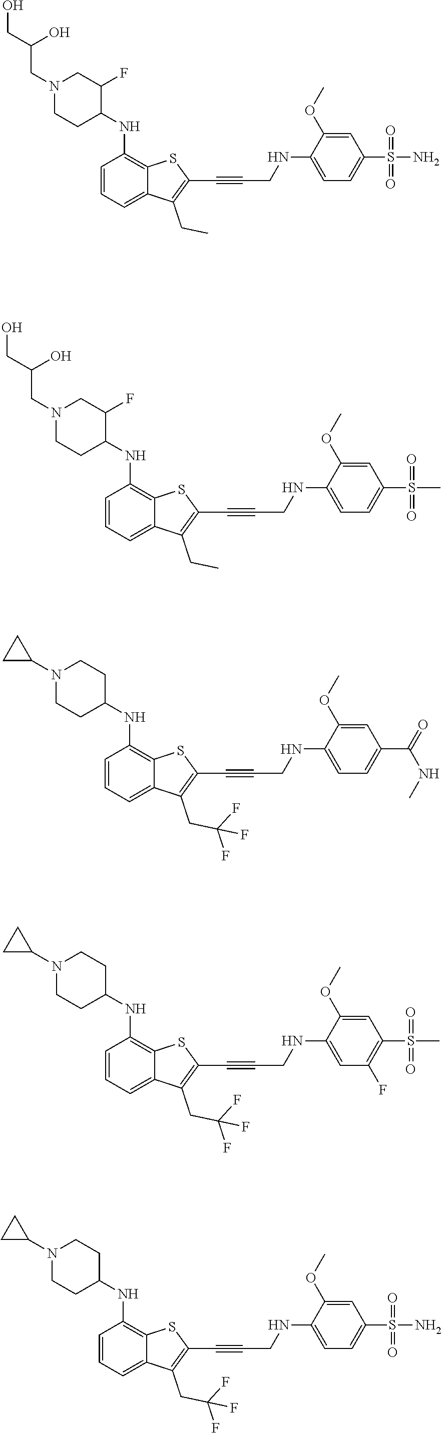 [SMILES: CCc1c(C#CCNc2ccc(S(N)(=O)=O)cc2OC)sc2c(NC3CCN(CC(O)CO)CC3F)cccc12 ; CCc1c(C#CCNc2ccc(S(C)(=O)=O)cc2OC)sc2c(NC3CCN(CC(O)CO)CC3F)cccc12 ; CNC(=O)c1ccc(NCC#Cc2sc3c(NC4CCN(C5CC5)CC4)cccc3c2CC(F)(F)F)c(OC)c1 ; COc1cc(S(C)(=O)=O)c(F)cc1NCC#Cc1sc2c(NC3CCN(C4CC4)CC3)cccc2c1CC(F)(F)F ; COc1cc(S(N)(=O)=O)ccc1NCC#Cc1sc2c(NC3CCN(C4CC4)CC3)cccc2c1CC(F)(F)F] 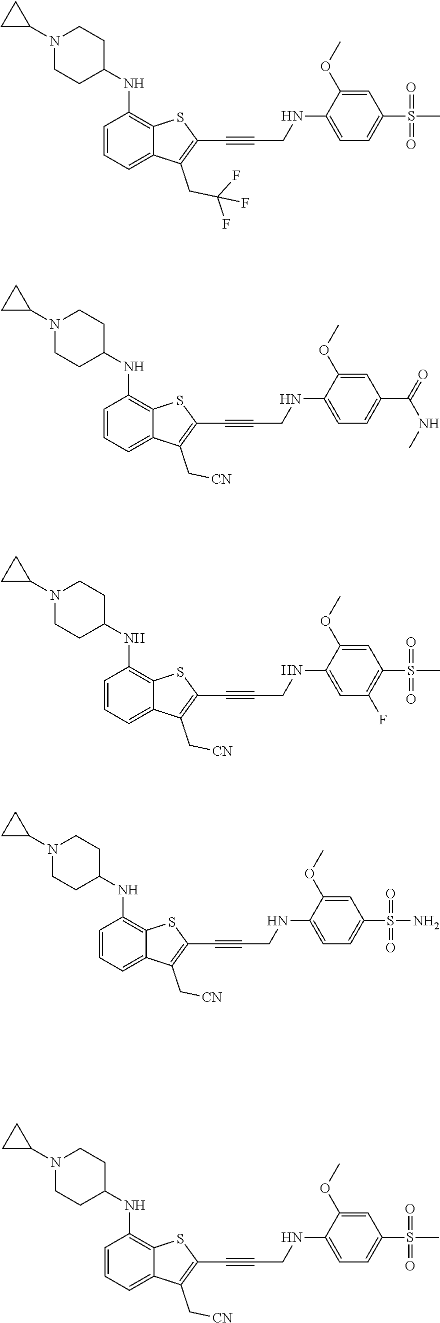 [SMILES: COc1cc(S(C)(=O)=O)ccc1NCC#Cc1sc2c(NC3CCN(C4CC4)CC3)cccc2c1CC(F)(F)F ; CNC(=O)c1ccc(NCC#Cc2sc3c(NC4CCN(C5CC5)CC4)cccc3c2CC#N)c(OC)c1 ; COc1cc(S(C)(=O)=O)c(F)cc1NCC#Cc1sc2c(NC3CCN(C4CC4)CC3)cccc2c1CC#N ; COc1cc(S(N)(=O)=O)ccc1NCC#Cc1sc2c(NC3CCN(C4CC4)CC3)cccc2c1CC#N ; COc1cc(S(C)(=O)=O)ccc1NCC#Cc1sc2c(NC3CCN(C4CC4)CC3)cccc2c1CC#N] 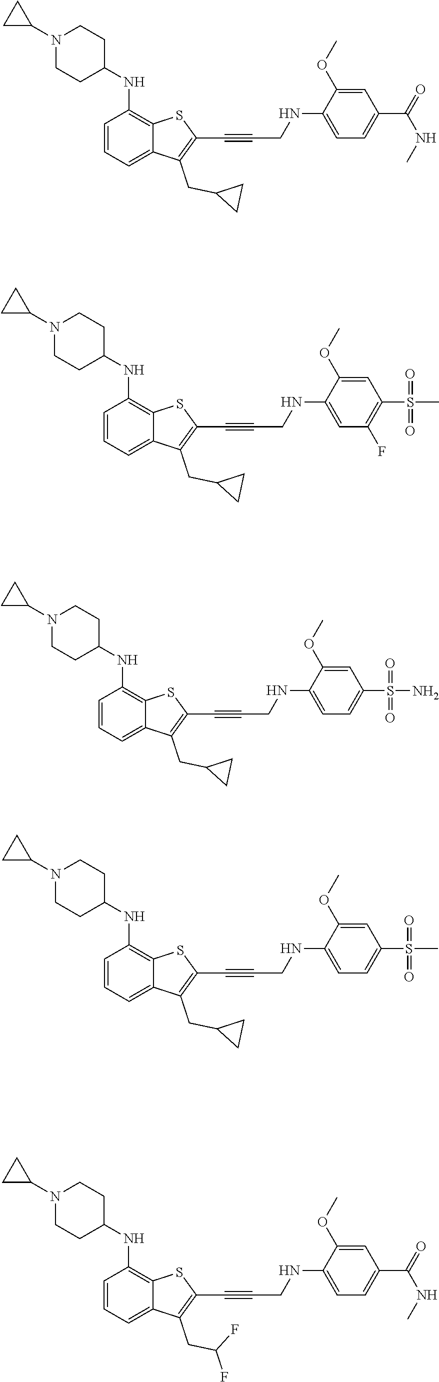 [SMILES: CNC(=O)c1ccc(NCC#Cc2sc3c(NC4CCN(C5CC5)CC4)cccc3c2CC2CC2)c(OC)c1 ; COc1cc(S(C)(=O)=O)c(F)cc1NCC#Cc1sc2c(NC3CCN(C4CC4)CC3)cccc2c1CC1CC1 ; COc1cc(S(N)(=O)=O)ccc1NCC#Cc1sc2c(NC3CCN(C4CC4)CC3)cccc2c1CC1CC1 ; COc1cc(S(C)(=O)=O)ccc1NCC#Cc1sc2c(NC3CCN(C4CC4)CC3)cccc2c1CC1CC1 ; CNC(=O)c1ccc(NCC#Cc2sc3c(NC4CCN(C5CC5)CC4)cccc3c2CC(F)F)c(OC)c1] 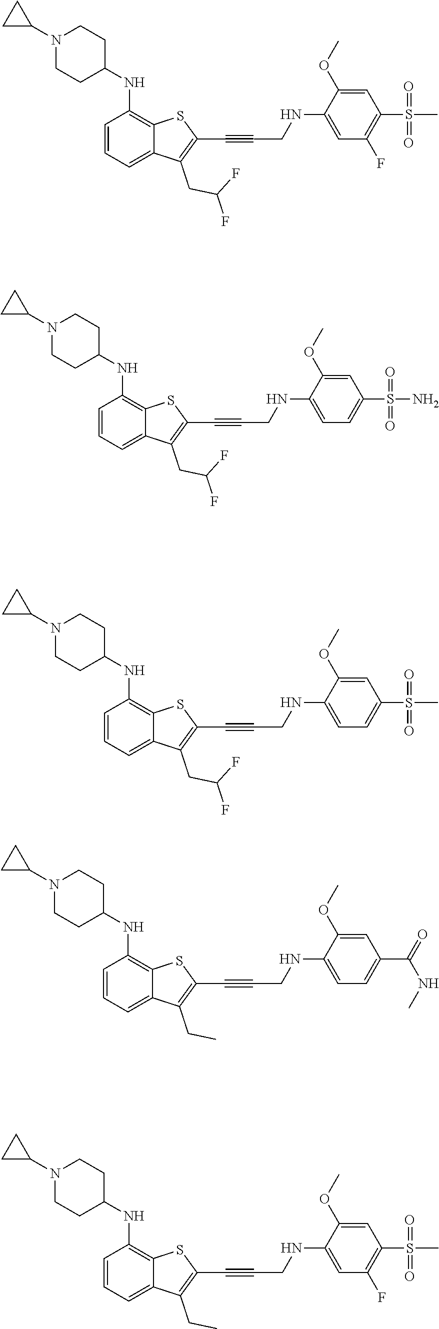 [SMILES: COc1cc(S(C)(=O)=O)c(F)cc1NCC#Cc1sc2c(NC3CCN(C4CC4)CC3)cccc2c1CC(F)F ; COc1cc(S(N)(=O)=O)ccc1NCC#Cc1sc2c(NC3CCN(C4CC4)CC3)cccc2c1CC(F)F ; COc1cc(S(C)(=O)=O)ccc1NCC#Cc1sc2c(NC3CCN(C4CC4)CC3)cccc2c1CC(F)F ; CCc1c(C#CCNc2ccc(C(=O)NC)cc2OC)sc2c(NC3CCN(C4CC4)CC3)cccc12 ; CCc1c(C#CCNc2cc(F)c(S(C)(=O)=O)cc2OC)sc2c(NC3CCN(C4CC4)CC3)cccc12] 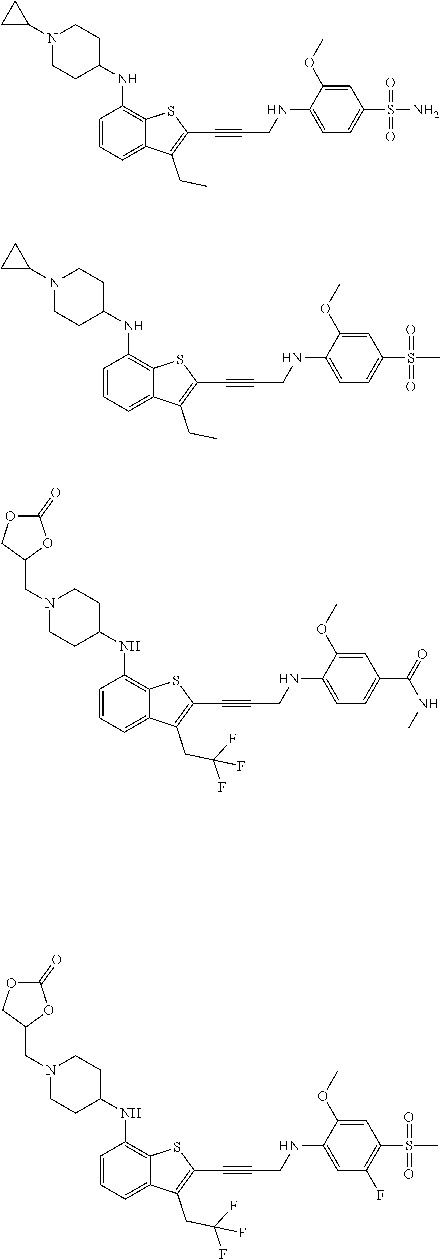 [SMILES: CCc1c(C#CCNc2ccc(S(N)(=O)=O)cc2OC)sc2c(NC3CCN(C4CC4)CC3)cccc12 ; CCc1c(C#CCNc2ccc(S(C)(=O)=O)cc2OC)sc2c(NC3CCN(C4CC4)CC3)cccc12 ; CNC(=O)c1ccc(NCC#Cc2sc3c(NC4CCN(CC5COC(=O)O5)CC4)cccc3c2CC(F)(F)F)c(OC)c1 ; COc1cc(S(C)(=O)=O)c(F)cc1NCC#Cc1sc2c(NC3CCN(CC4COC(=O)O4)CC3)cccc2c1CC(F)(F)F] 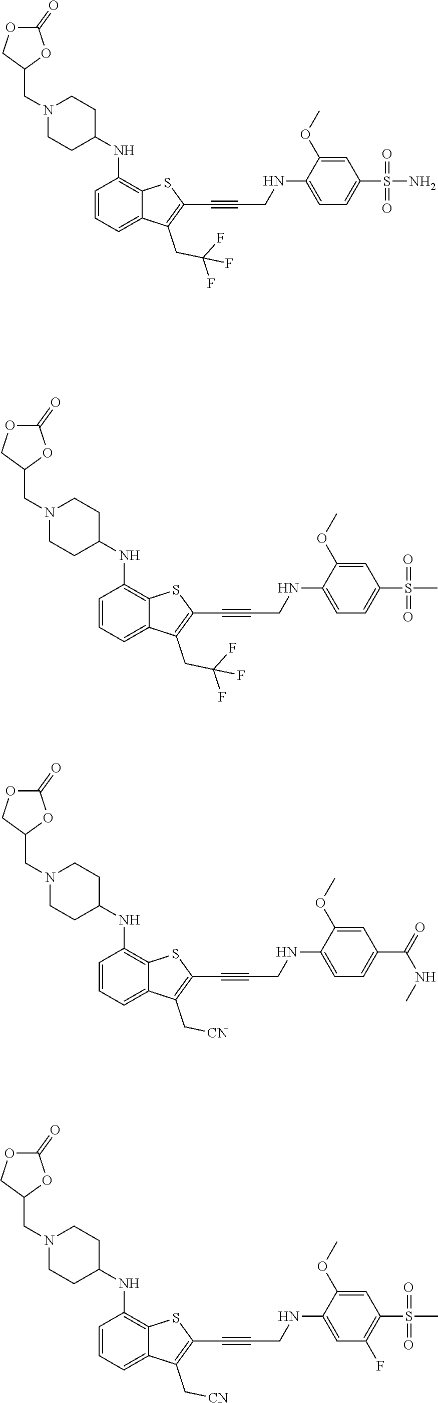 [SMILES: COc1cc(S(N)(=O)=O)ccc1NCC#Cc1sc2c(NC3CCN(CC4COC(=O)O4)CC3)cccc2c1CC(F)(F)F ; COc1cc(S(C)(=O)=O)ccc1NCC#Cc1sc2c(NC3CCN(CC4COC(=O)O4)CC3)cccc2c1CC(F)(F)F ; CNC(=O)c1ccc(NCC#Cc2sc3c(NC4CCN(CC5COC(=O)O5)CC4)cccc3c2CC#N)c(OC)c1 ; COc1cc(S(C)(=O)=O)c(F)cc1NCC#Cc1sc2c(NC3CCN(CC4COC(=O)O4)CC3)cccc2c1CC#N] 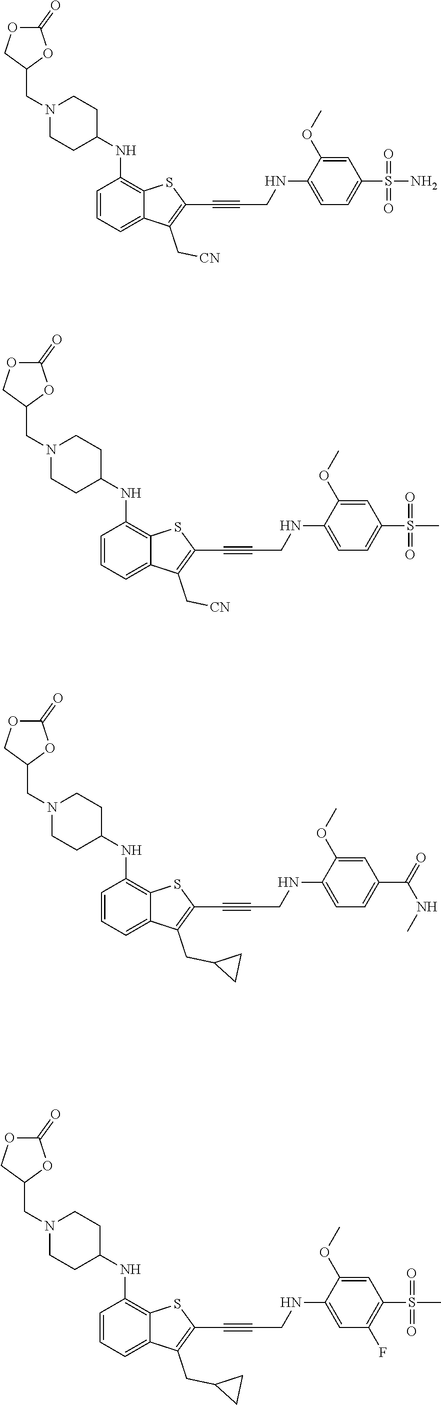 [SMILES: COc1cc(S(N)(=O)=O)ccc1NCC#Cc1sc2c(NC3CCN(CC4COC(=O)O4)CC3)cccc2c1CC#N ; COc1cc(S(C)(=O)=O)ccc1NCC#Cc1sc2c(NC3CCN(CC4COC(=O)O4)CC3)cccc2c1CC#N ; CNC(=O)c1ccc(NCC#Cc2sc3c(NC4CCN(CC5COC(=O)O5)CC4)cccc3c2CC2CC2)c(OC)c1 ; COc1cc(S(C)(=O)=O)c(F)cc1NCC#Cc1sc2c(NC3CCN(CC4COC(=O)O4)CC3)cccc2c1CC1CC1] 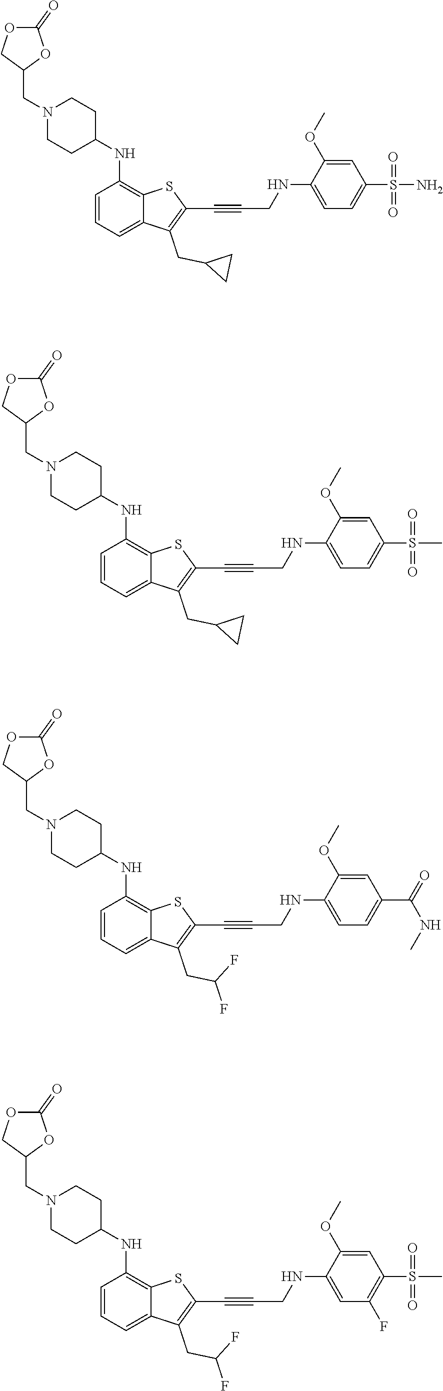 [SMILES: COc1cc(S(N)(=O)=O)ccc1NCC#Cc1sc2c(NC3CCN(CC4COC(=O)O4)CC3)cccc2c1CC1CC1 ; COc1cc(S(C)(=O)=O)ccc1NCC#Cc1sc2c(NC3CCN(CC4COC(=O)O4)CC3)cccc2c1CC1CC1 ; CNC(=O)c1ccc(NCC#Cc2sc3c(NC4CCN(CC5COC(=O)O5)CC4)cccc3c2CC(F)F)c(OC)c1 ; COc1cc(S(C)(=O)=O)c(F)cc1NCC#Cc1sc2c(NC3CCN(CC4COC(=O)O4)CC3)cccc2c1CC(F)F] 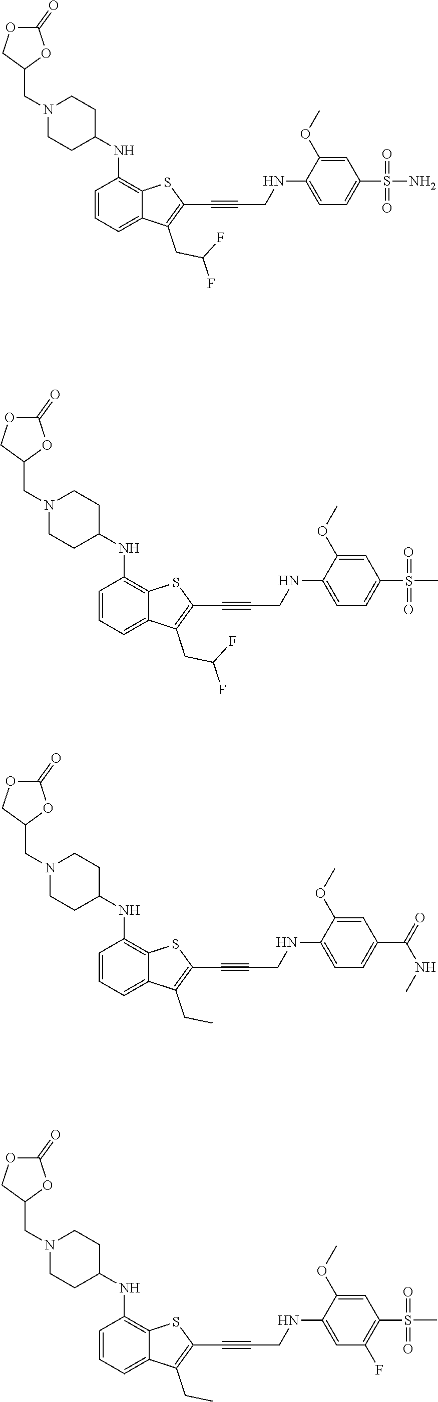 [SMILES: COc1cc(S(N)(=O)=O)ccc1NCC#Cc1sc2c(NC3CCN(CC4COC(=O)O4)CC3)cccc2c1CC(F)F ; COc1cc(S(C)(=O)=O)ccc1NCC#Cc1sc2c(NC3CCN(CC4COC(=O)O4)CC3)cccc2c1CC(F)F ; CCc1c(C#CCNc2ccc(C(=O)NC)cc2OC)sc2c(NC3CCN(CC4COC(=O)O4)CC3)cccc12 ; CCc1c(C#CCNc2cc(F)c(S(C)(=O)=O)cc2OC)sc2c(NC3CCN(CC4COC(=O)O4)CC3)cccc12] 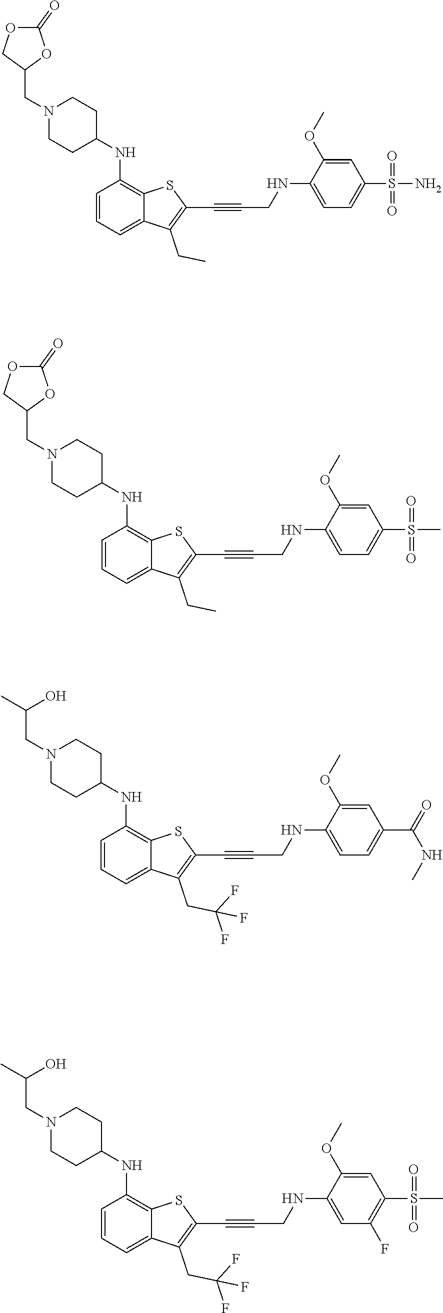 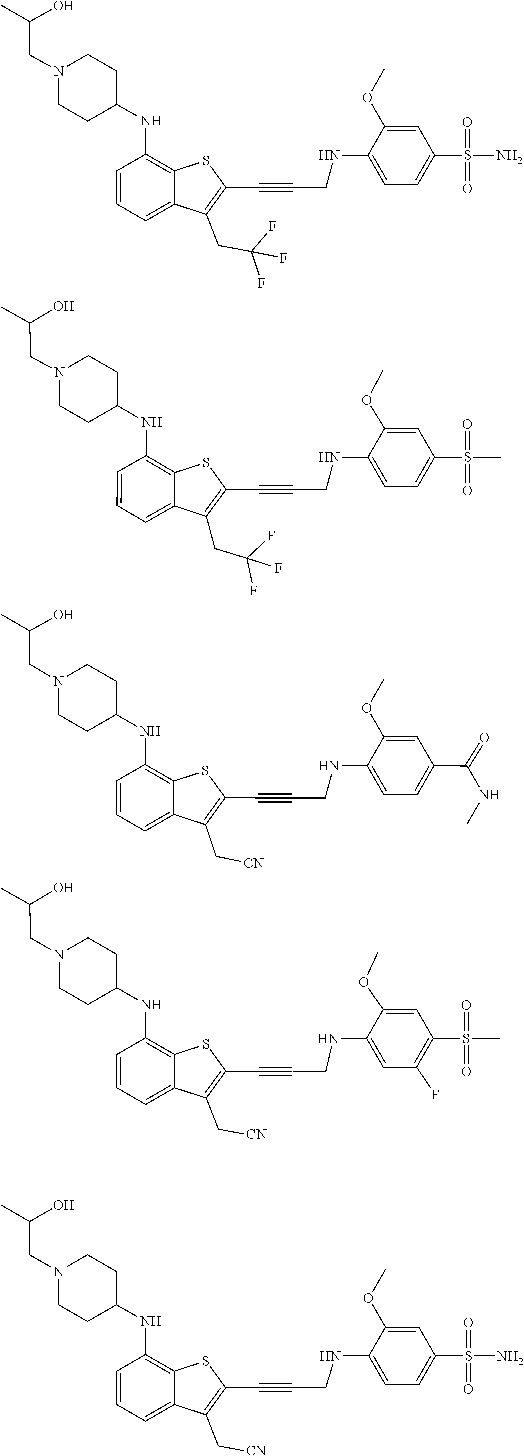 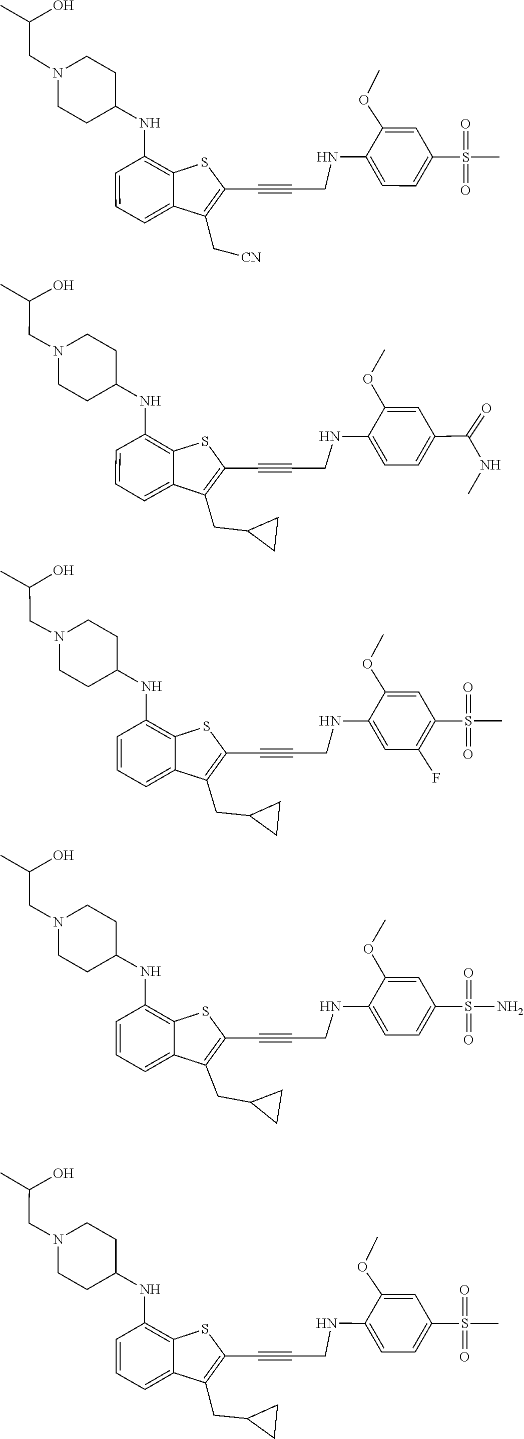 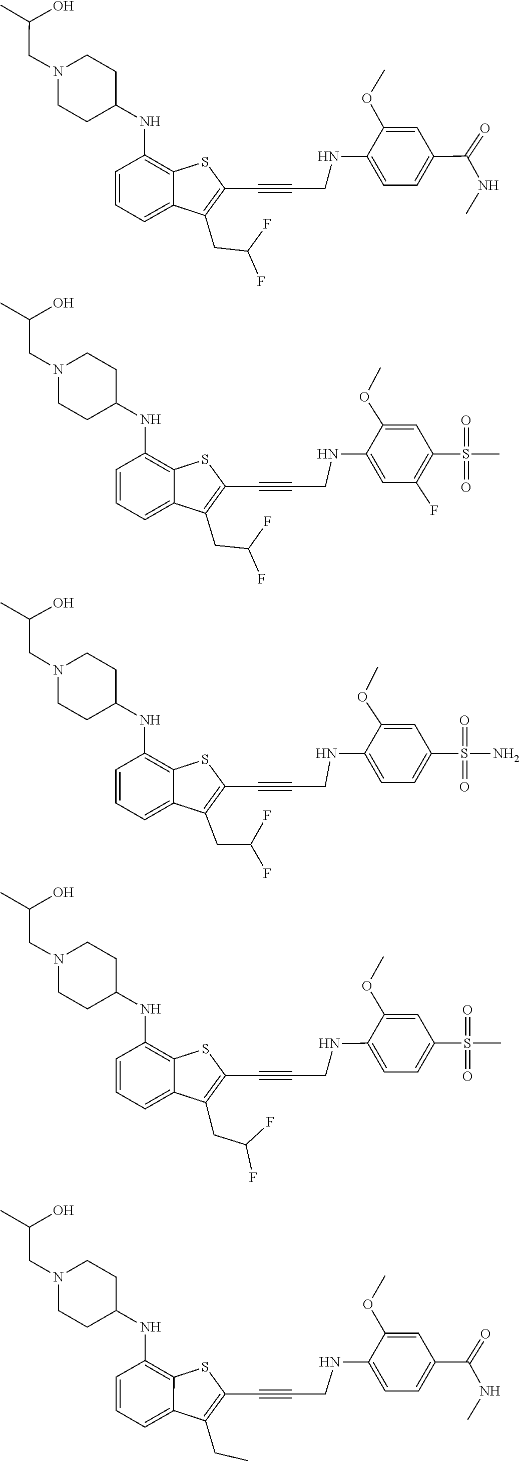 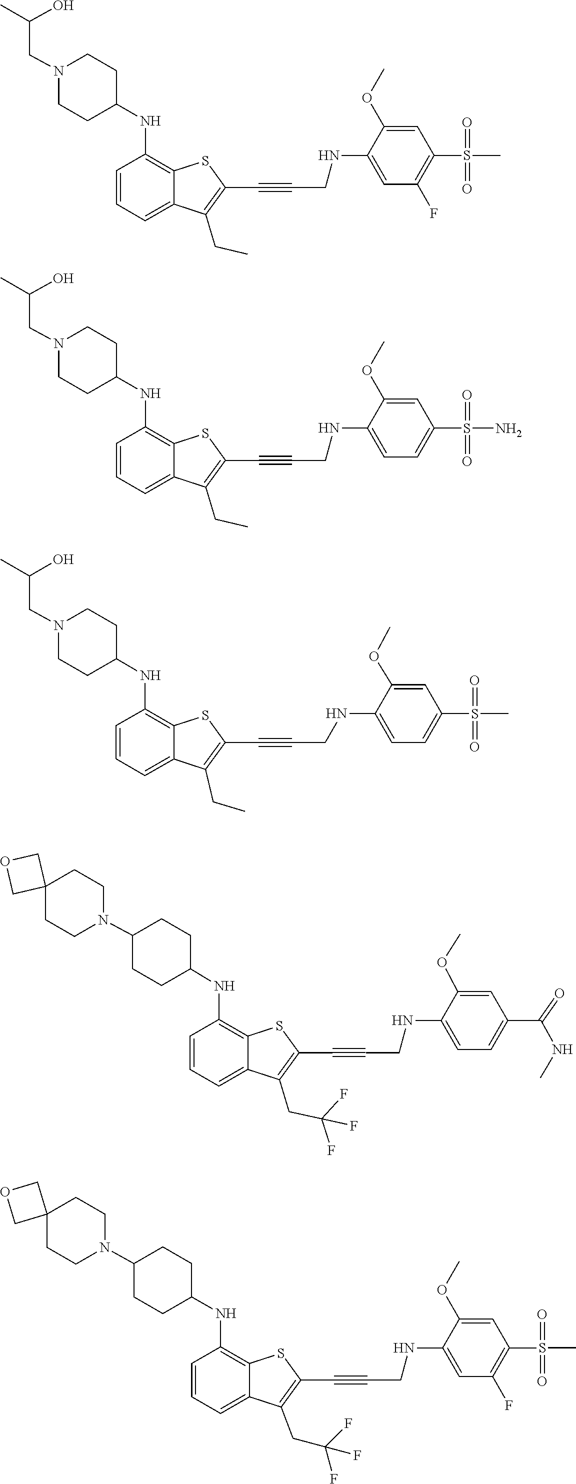 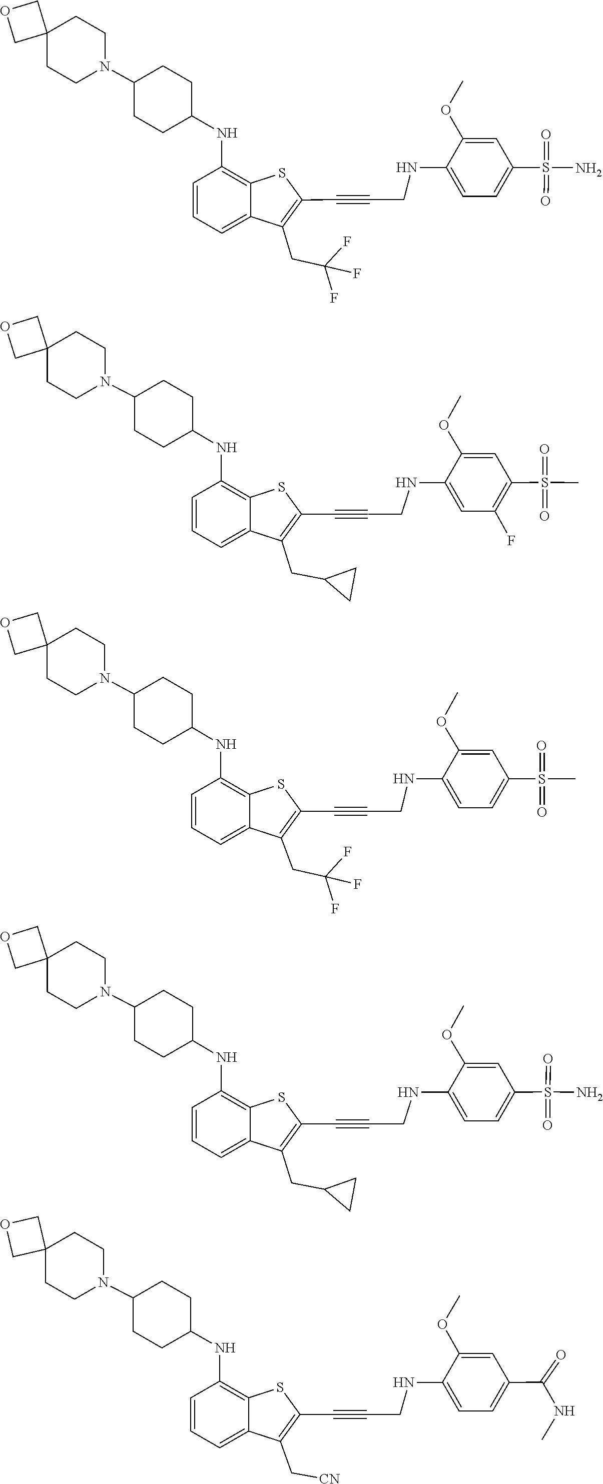 [SMILES: COc1cc(S(N)(=O)=O)ccc1NCC#Cc1sc2c(NC3CCC(N4CCC5(CC4)COC5)CC3)cccc2c1CC(F)(F)F ; COc1cc(S(C)(=O)=O)c(F)cc1NCC#Cc1sc2c(NC3CCC(N4CCC5(CC4)COC5)CC3)cccc2c1CC1CC1 ; COc1cc(S(C)(=O)=O)ccc1NCC#Cc1sc2c(NC3CCC(N4CCC5(CC4)COC5)CC3)cccc2c1CC(F)(F)F ; COc1cc(S(N)(=O)=O)ccc1NCC#Cc1sc2c(NC3CCC(N4CCC5(CC4)COC5)CC3)cccc2c1CC1CC1 ; CNC(=O)c1ccc(NCC#Cc2sc3c(NC4CCC(N5CCC6(CC5)COC6)CC4)cccc3c2CC#N)c(OC)c1] 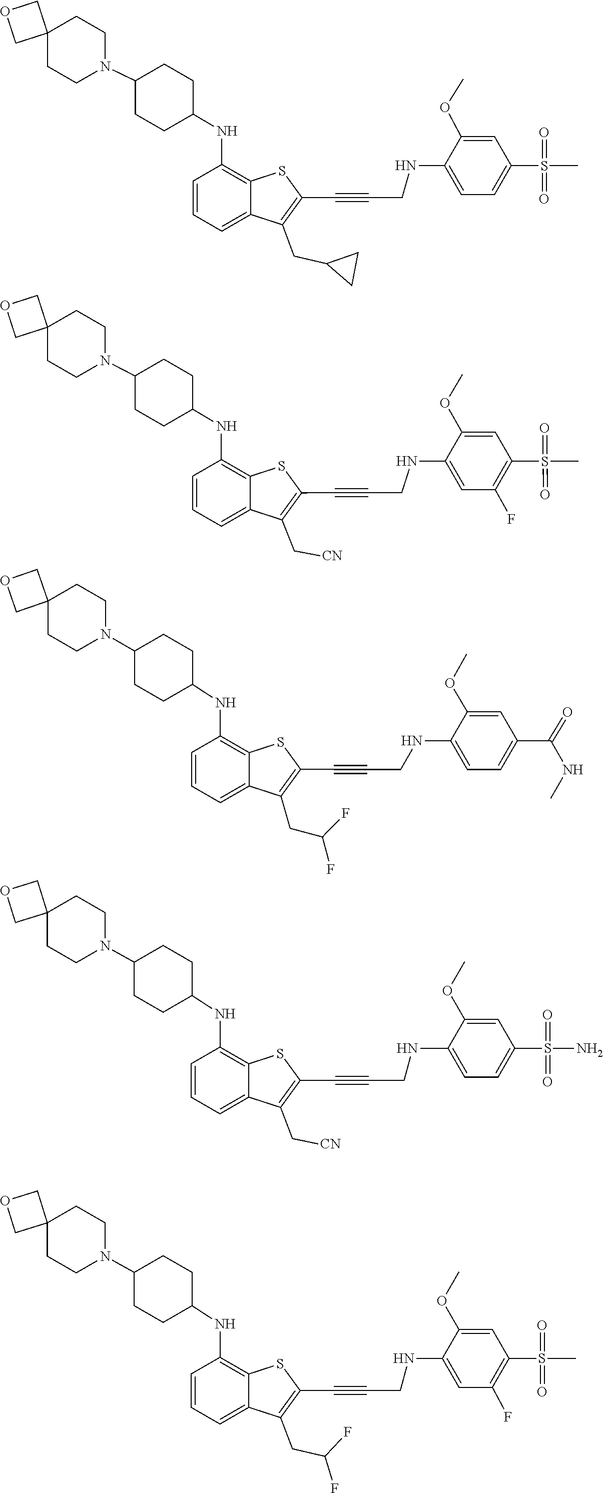 [SMILES: COc1cc(S(C)(=O)=O)ccc1NCC#Cc1sc2c(NC3CCC(N4CCC5(CC4)COC5)CC3)cccc2c1CC1CC1 ; COc1cc(S(C)(=O)=O)c(F)cc1NCC#Cc1sc2c(NC3CCC(N4CCC5(CC4)COC5)CC3)cccc2c1CC#N ; CNC(=O)c1ccc(NCC#Cc2sc3c(NC4CCC(N5CCC6(CC5)COC6)CC4)cccc3c2CC(F)F)c(OC)c1 ; COc1cc(S(N)(=O)=O)ccc1NCC#Cc1sc2c(NC3CCC(N4CCC5(CC4)COC5)CC3)cccc2c1CC#N ; COc1cc(S(C)(=O)=O)c(F)cc1NCC#Cc1sc2c(NC3CCC(N4CCC5(CC4)COC5)CC3)cccc2c1CC(F)F] 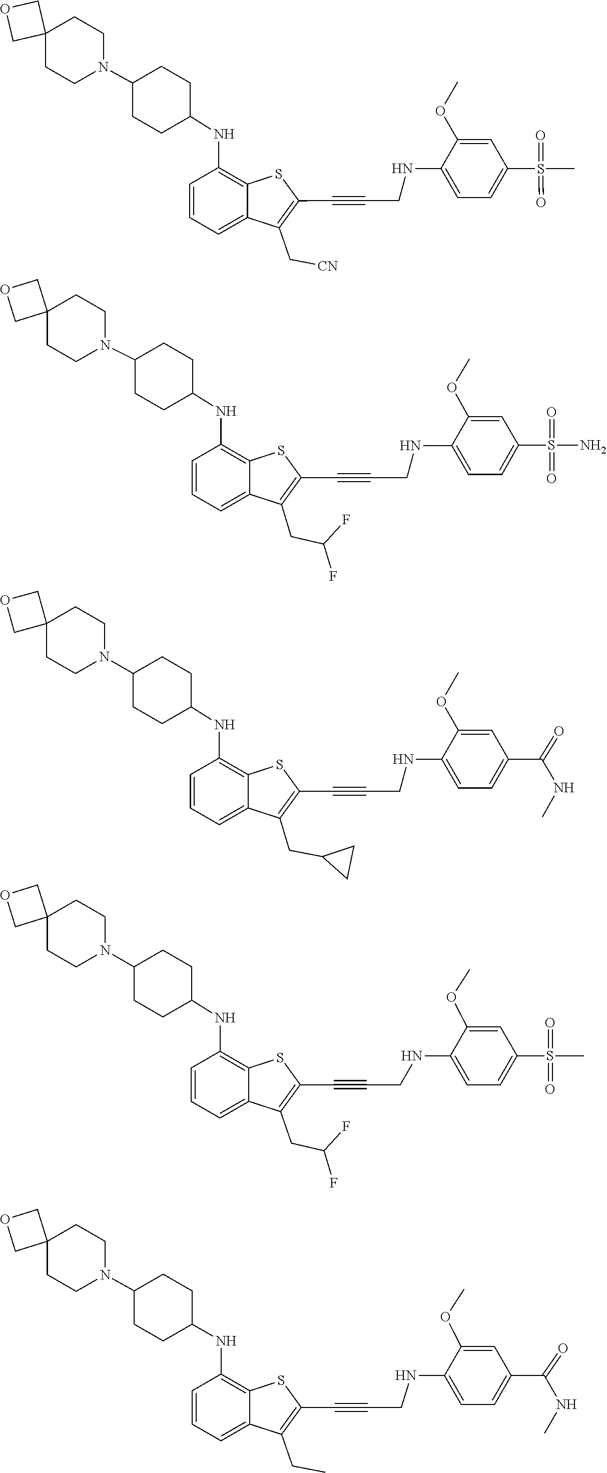 [SMILES: COc1cc(S(C)(=O)=O)ccc1NCC#Cc1sc2c(NC3CCC(N4CCC5(CC4)COC5)CC3)cccc2c1CC#N ; COc1cc(S(N)(=O)=O)ccc1NCC#Cc1sc2c(NC3CCC(N4CCC5(CC4)COC5)CC3)cccc2c1CC(F)F ; CNC(=O)c1ccc(NCC#Cc2sc3c(NC4CCC(N5CCC6(CC5)COC6)CC4)cccc3c2CC2CC2)c(OC)c1 ; COc1cc(S(C)(=O)=O)ccc1NCC#Cc1sc2c(NC3CCC(N4CCC5(CC4)COC5)CC3)cccc2c1CC(F)F ; CCc1c(C#CCNc2ccc(C(=O)NC)cc2OC)sc2c(NC3CCC(N4CCC5(CC4)COC5)CC3)cccc12] 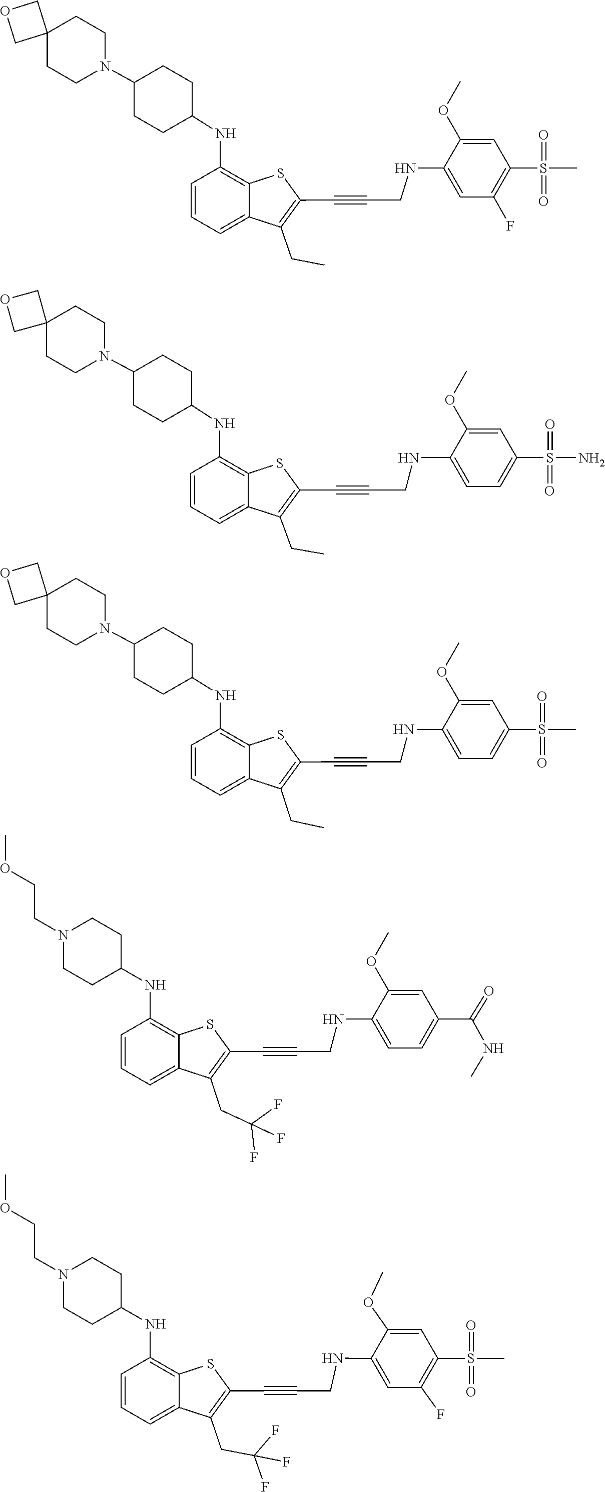 [SMILES: CCc1c(C#CCNc2cc(F)c(S(C)(=O)=O)cc2OC)sc2c(NC3CCC(N4CCC5(CC4)COC5)CC3)cccc12 ; CCc1c(C#CCNc2ccc(S(N)(=O)=O)cc2OC)sc2c(NC3CCC(N4CCC5(CC4)COC5)CC3)cccc12 ; CCc1c(C#CCNc2ccc(S(C)(=O)=O)cc2OC)sc2c(NC3CCC(N4CCC5(CC4)COC5)CC3)cccc12 ; CNC(=O)c1ccc(NCC#Cc2sc3c(NC4CCN(CCOC)CC4)cccc3c2CC(F)(F)F)c(OC)c1 ; COCCN1CCC(Nc2cccc3c(CC(F)(F)F)c(C#CCNc4cc(F)c(S(C)(=O)=O)cc4OC)sc23)CC1] 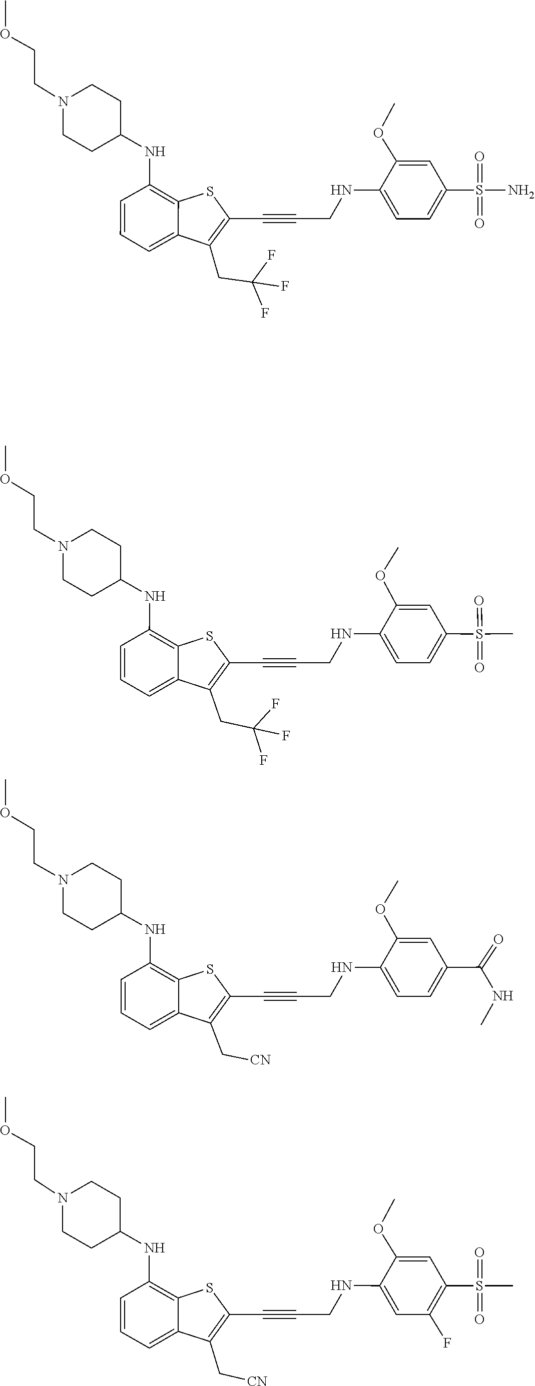 [SMILES: COCCN1CCC(Nc2cccc3c(CC(F)(F)F)c(C#CCNc4ccc(S(N)(=O)=O)cc4OC)sc23)CC1 ; COCCN1CCC(Nc2cccc3c(CC(F)(F)F)c(C#CCNc4ccc(S(C)(=O)=O)cc4OC)sc23)CC1 ; CNC(=O)c1ccc(NCC#Cc2sc3c(NC4CCN(CCOC)CC4)cccc3c2CC#N)c(OC)c1 ; COCCN1CCC(Nc2cccc3c(CC#N)c(C#CCNc4cc(F)c(S(C)(=O)=O)cc4OC)sc23)CC1] 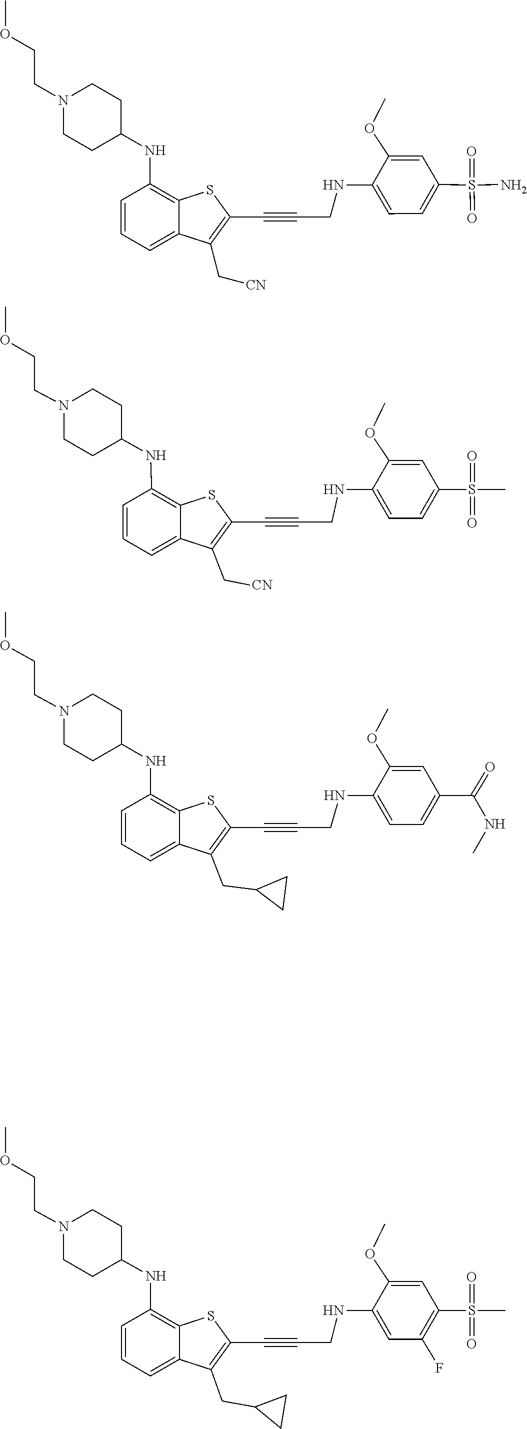 [SMILES: COCCN1CCC(Nc2cccc3c(CC#N)c(C#CCNc4ccc(S(N)(=O)=O)cc4OC)sc23)CC1 ; COCCN1CCC(Nc2cccc3c(CC#N)c(C#CCNc4ccc(S(C)(=O)=O)cc4OC)sc23)CC1 ; CNC(=O)c1ccc(NCC#Cc2sc3c(NC4CCN(CCOC)CC4)cccc3c2CC2CC2)c(OC)c1 ; COCCN1CCC(Nc2cccc3c(CC4CC4)c(C#CCNc4cc(F)c(S(C)(=O)=O)cc4OC)sc23)CC1] 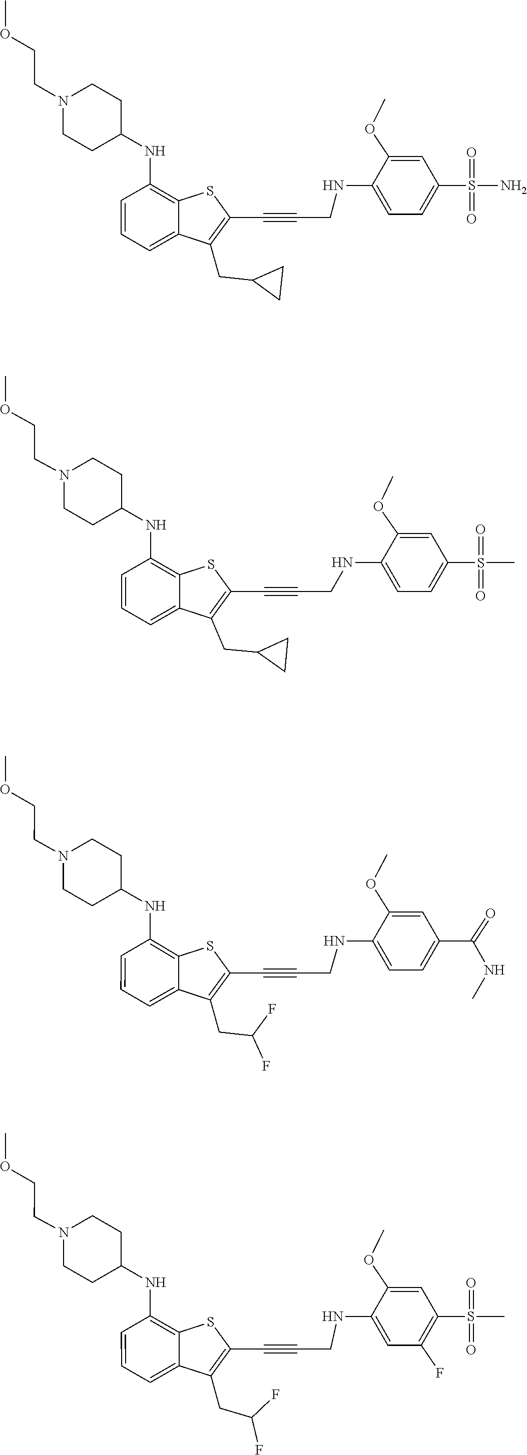 [SMILES: COCCN1CCC(Nc2cccc3c(CC4CC4)c(C#CCNc4ccc(S(N)(=O)=O)cc4OC)sc23)CC1 ; COCCN1CCC(Nc2cccc3c(CC4CC4)c(C#CCNc4ccc(S(C)(=O)=O)cc4OC)sc23)CC1 ; CNC(=O)c1ccc(NCC#Cc2sc3c(NC4CCN(CCOC)CC4)cccc3c2CC(F)F)c(OC)c1 ; COCCN1CCC(Nc2cccc3c(CC(F)F)c(C#CCNc4cc(F)c(S(C)(=O)=O)cc4OC)sc23)CC1] 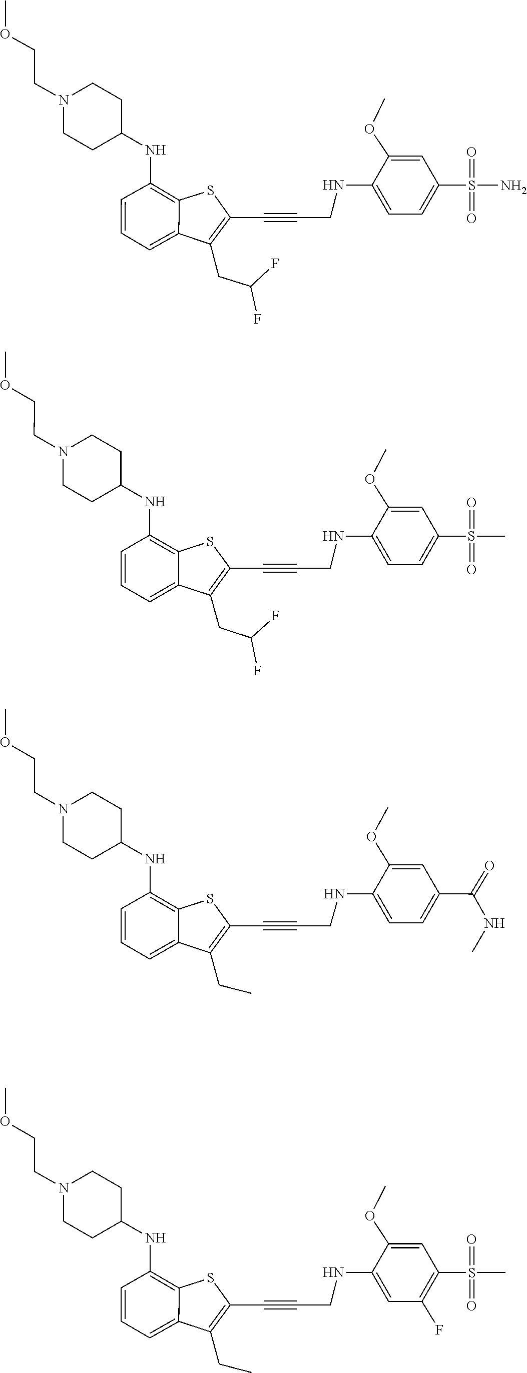 [SMILES: COCCN1CCC(Nc2cccc3c(CC(F)F)c(C#CCNc4ccc(S(N)(=O)=O)cc4OC)sc23)CC1 ; COCCN1CCC(Nc2cccc3c(CC(F)F)c(C#CCNc4ccc(S(C)(=O)=O)cc4OC)sc23)CC1 ; CCc1c(C#CCNc2ccc(C(=O)NC)cc2OC)sc2c(NC3CCN(CCOC)CC3)cccc12 ; CCc1c(C#CCNc2cc(F)c(S(C)(=O)=O)cc2OC)sc2c(NC3CCN(CCOC)CC3)cccc12] 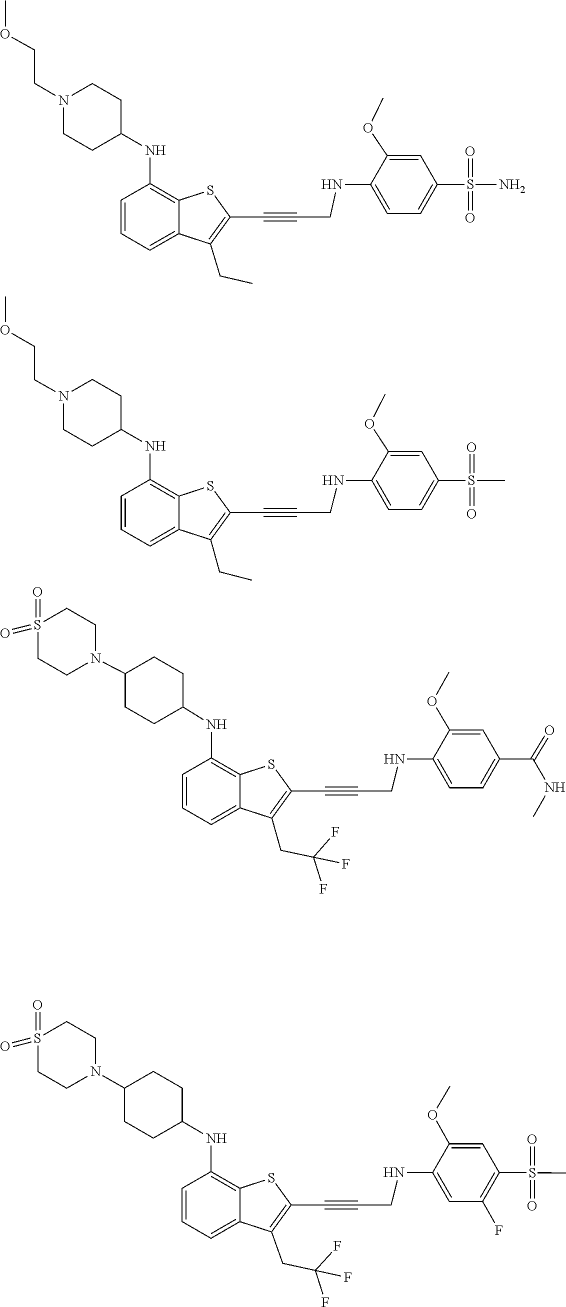 [SMILES: CCc1c(C#CCNc2ccc(S(N)(=O)=O)cc2OC)sc2c(NC3CCN(CCOC)CC3)cccc12 ; CCc1c(C#CCNc2ccc(S(C)(=O)=O)cc2OC)sc2c(NC3CCN(CCOC)CC3)cccc12 ; CNC(=O)c1ccc(NCC#Cc2sc3c(NC4CCC(N5CCS(=O)(=O)CC5)CC4)cccc3c2CC(F)(F)F)c(OC)c1 ; COc1cc(S(C)(=O)=O)c(F)cc1NCC#Cc1sc2c(NC3CCC(N4CCS(=O)(=O)CC4)CC3)cccc2c1CC(F)(F)F] 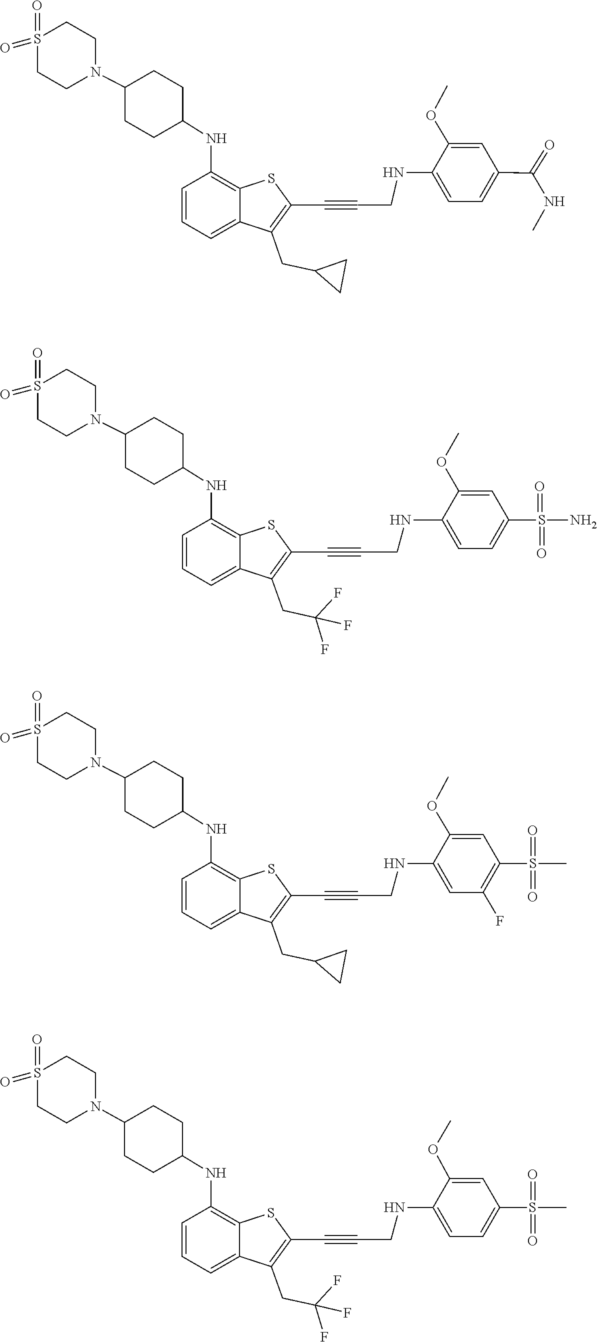 [SMILES: CNC(=O)c1ccc(NCC#Cc2sc3c(NC4CCC(N5CCS(=O)(=O)CC5)CC4)cccc3c2CC2CC2)c(OC)c1 ; COc1cc(S(N)(=O)=O)ccc1NCC#Cc1sc2c(NC3CCC(N4CCS(=O)(=O)CC4)CC3)cccc2c1CC(F)(F)F ; COc1cc(S(C)(=O)=O)c(F)cc1NCC#Cc1sc2c(NC3CCC(N4CCS(=O)(=O)CC4)CC3)cccc2c1CC1CC1 ; COc1cc(S(C)(=O)=O)ccc1NCC#Cc1sc2c(NC3CCC(N4CCS(=O)(=O)CC4)CC3)cccc2c1CC(F)(F)F] 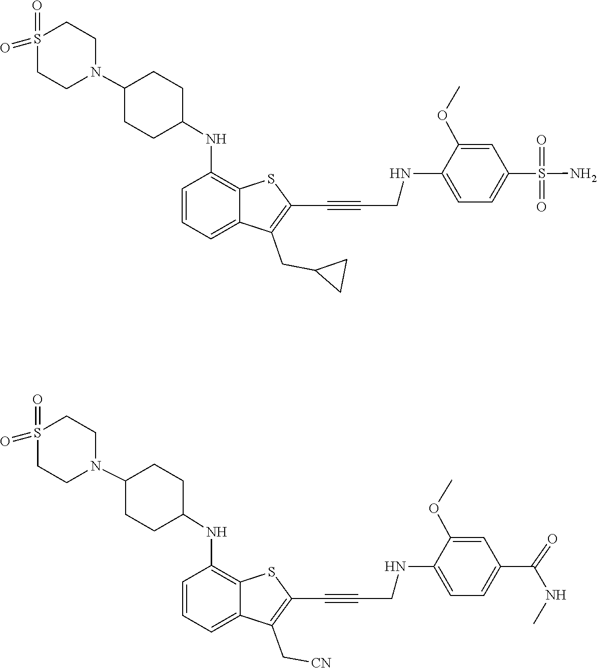 [SMILES: COc1cc(S(N)(=O)=O)ccc1NCC#Cc1sc2c(NC3CCC(N4CCS(=O)(=O)CC4)CC3)cccc2c1CC1CC1 ; CNC(=O)c1ccc(NCC#Cc2sc3c(NC4CCC(N5CCS(=O)(=O)CC5)CC4)cccc3c2CC#N)c(OC)c1] 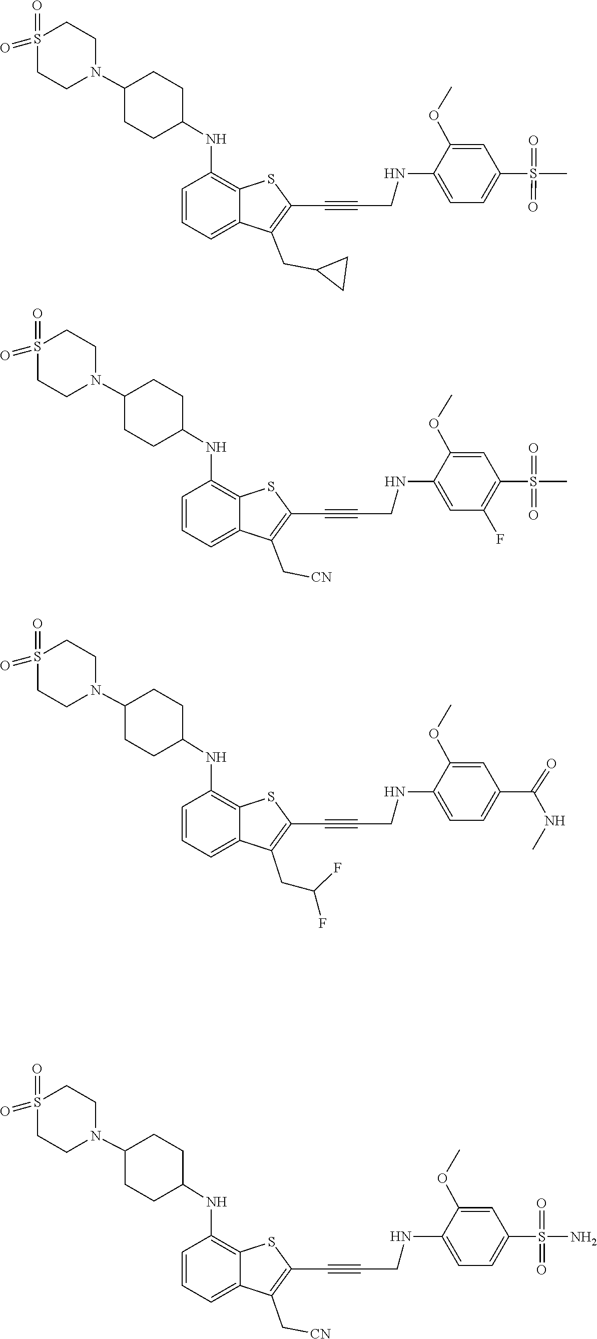 [SMILES: COc1cc(S(C)(=O)=O)ccc1NCC#Cc1sc2c(NC3CCC(N4CCS(=O)(=O)CC4)CC3)cccc2c1CC1CC1 ; COc1cc(S(C)(=O)=O)c(F)cc1NCC#Cc1sc2c(NC3CCC(N4CCS(=O)(=O)CC4)CC3)cccc2c1CC#N ; CNC(=O)c1ccc(NCC#Cc2sc3c(NC4CCC(N5CCS(=O)(=O)CC5)CC4)cccc3c2CC(F)F)c(OC)c1 ; COc1cc(S(N)(=O)=O)ccc1NCC#Cc1sc2c(NC3CCC(N4CCS(=O)(=O)CC4)CC3)cccc2c1CC#N] 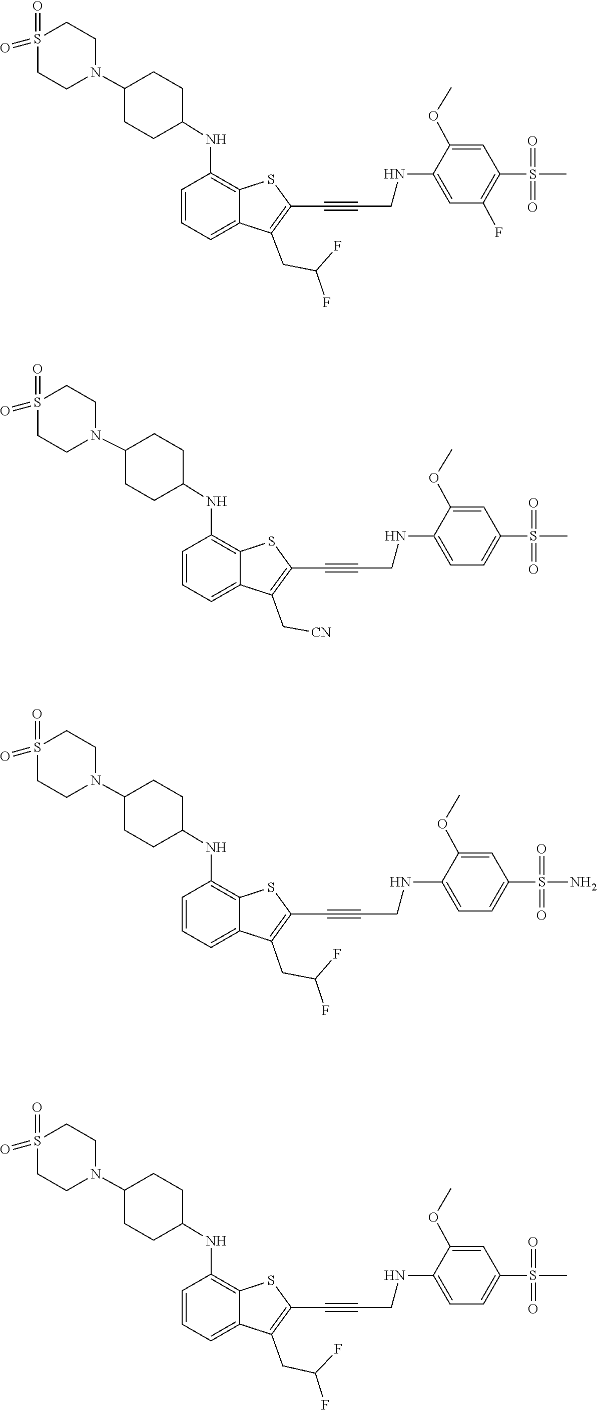 [SMILES: COc1cc(S(C)(=O)=O)c(F)cc1NCC#Cc1sc2c(NC3CCC(N4CCS(=O)(=O)CC4)CC3)cccc2c1CC(F)F ; COc1cc(S(C)(=O)=O)ccc1NCC#Cc1sc2c(NC3CCC(N4CCS(=O)(=O)CC4)CC3)cccc2c1CC#N ; COc1cc(S(N)(=O)=O)ccc1NCC#Cc1sc2c(NC3CCC(N4CCS(=O)(=O)CC4)CC3)cccc2c1CC(F)F ; COc1cc(S(C)(=O)=O)ccc1NCC#Cc1sc2c(NC3CCC(N4CCS(=O)(=O)CC4)CC3)cccc2c1CC(F)F] 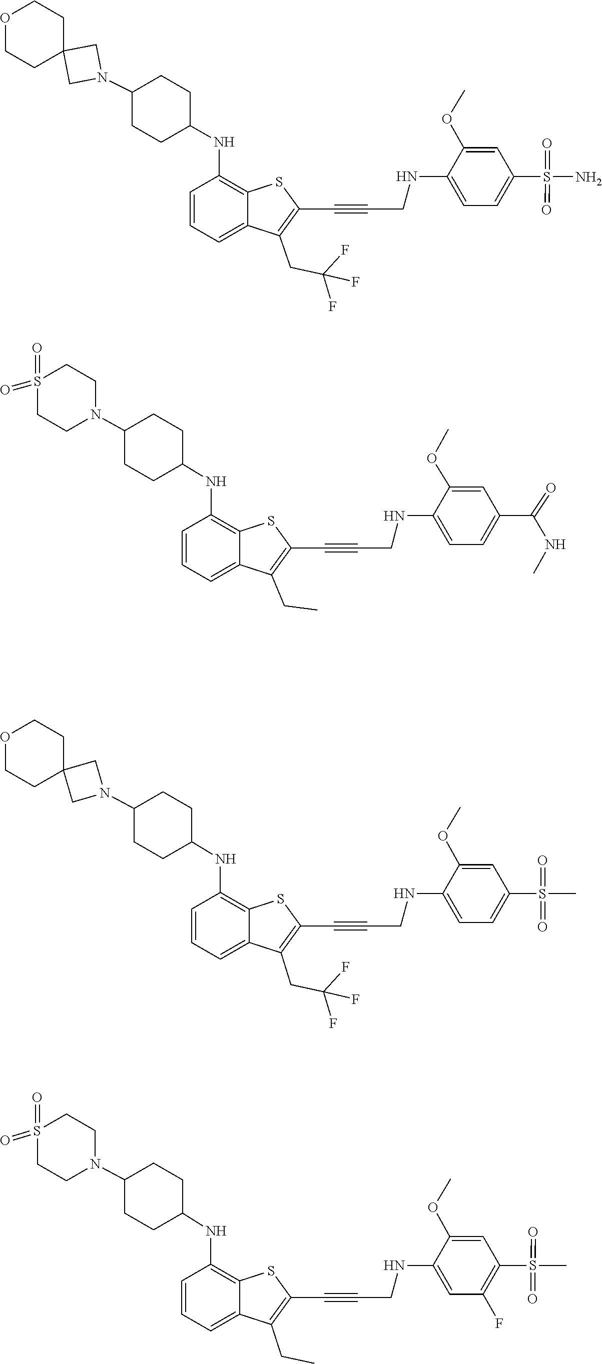 [SMILES: COc1cc(S(N)(=O)=O)ccc1NCC#Cc1sc2c(NC3CCC(N4CC5(CCOCC5)C4)CC3)cccc2c1CC(F)(F)F ; CCc1c(C#CCNc2ccc(C(=O)NC)cc2OC)sc2c(NC3CCC(N4CCS(=O)(=O)CC4)CC3)cccc12 ; COc1cc(S(C)(=O)=O)ccc1NCC#Cc1sc2c(NC3CCC(N4CC5(CCOCC5)C4)CC3)cccc2c1CC(F)(F)F ; CCc1c(C#CCNc2cc(F)c(S(C)(=O)=O)cc2OC)sc2c(NC3CCC(N4CCS(=O)(=O)CC4)CC3)cccc12] 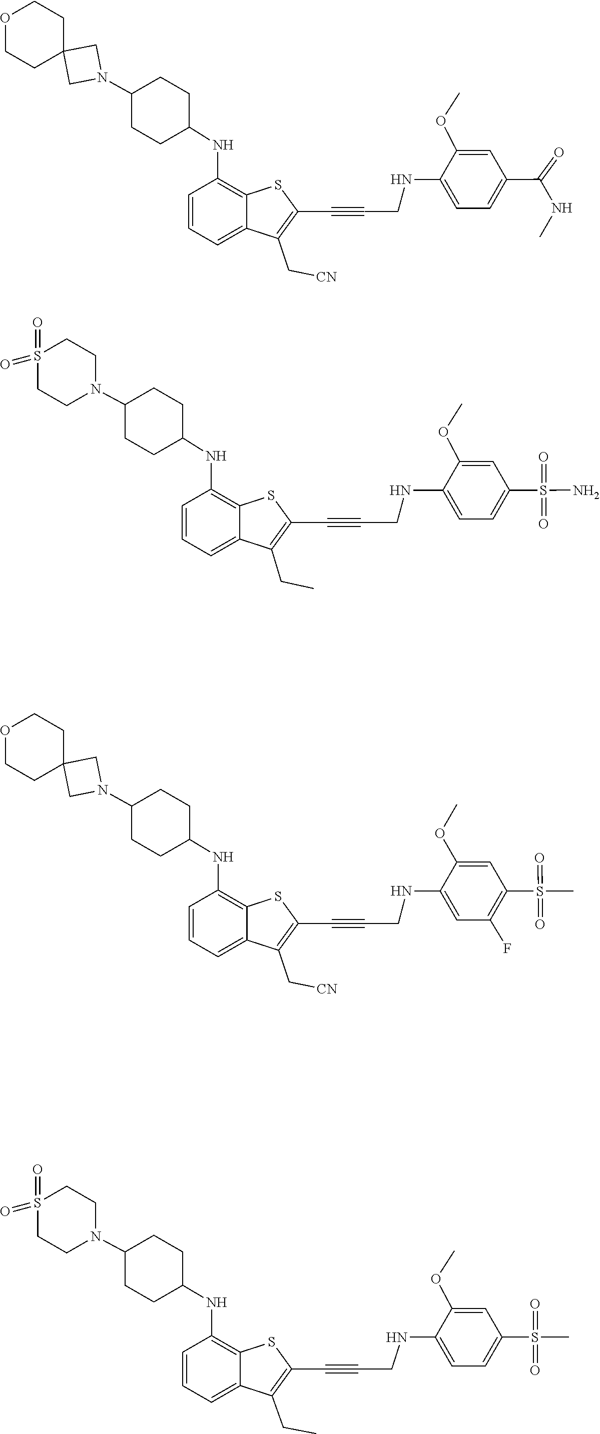 [SMILES: CNC(=O)c1ccc(NCC#Cc2sc3c(NC4CCC(N5CC6(CCOCC6)C5)CC4)cccc3c2CC#N)c(OC)c1 ; CCc1c(C#CCNc2ccc(S(N)(=O)=O)cc2OC)sc2c(NC3CCC(N4CCS(=O)(=O)CC4)CC3)cccc12 ; COc1cc(S(C)(=O)=O)c(F)cc1NCC#Cc1sc2c(NC3CCC(N4CC5(CCOCC5)C4)CC3)cccc2c1CC#N ; CCc1c(C#CCNc2ccc(S(C)(=O)=O)cc2OC)sc2c(NC3CCC(N4CCS(=O)(=O)CC4)CC3)cccc12] 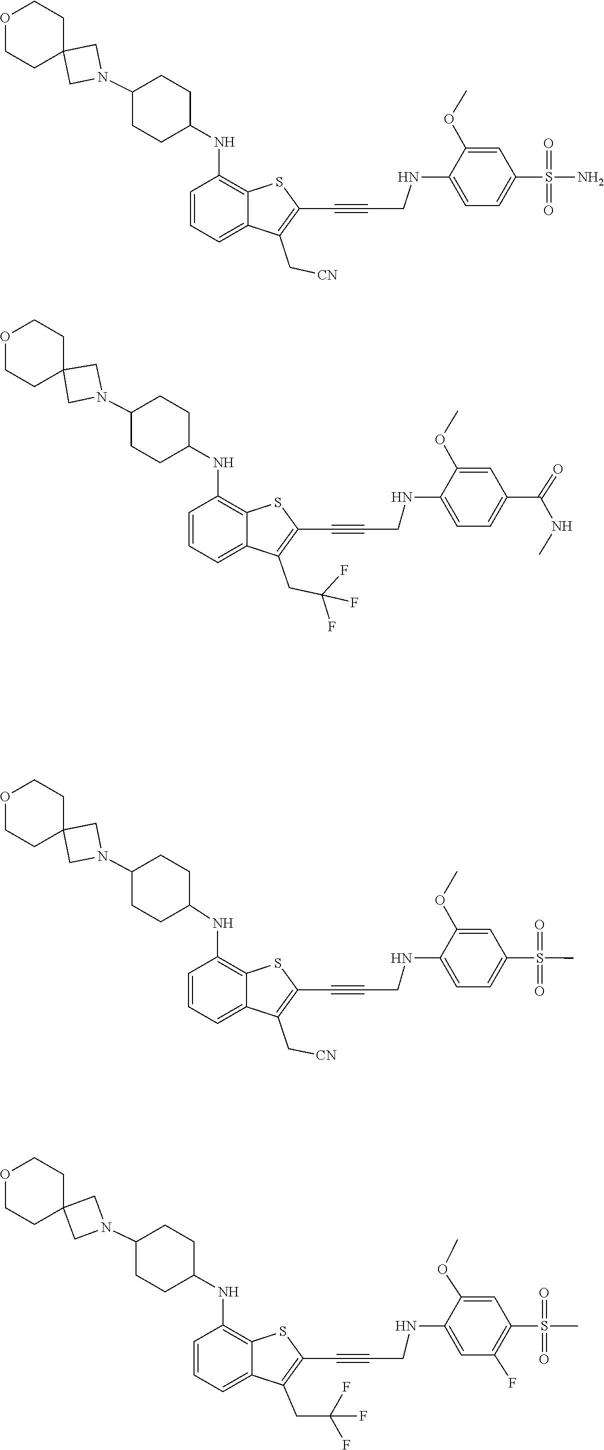 [SMILES: COc1cc(S(N)(=O)=O)ccc1NCC#Cc1sc2c(NC3CCC(N4CC5(CCOCC5)C4)CC3)cccc2c1CC#N ; CNC(=O)c1ccc(NCC#Cc2sc3c(NC4CCC(N5CC6(CCOCC6)C5)CC4)cccc3c2CC(F)(F)F)c(OC)c1 ; COc1cc(S(C)(=O)=O)ccc1NCC#Cc1sc2c(NC3CCC(N4CC5(CCOCC5)C4)CC3)cccc2c1CC#N ; COc1cc(S(C)(=O)=O)c(F)cc1NCC#Cc1sc2c(NC3CCC(N4CC5(CCOCC5)C4)CC3)cccc2c1CC(F)(F)F] 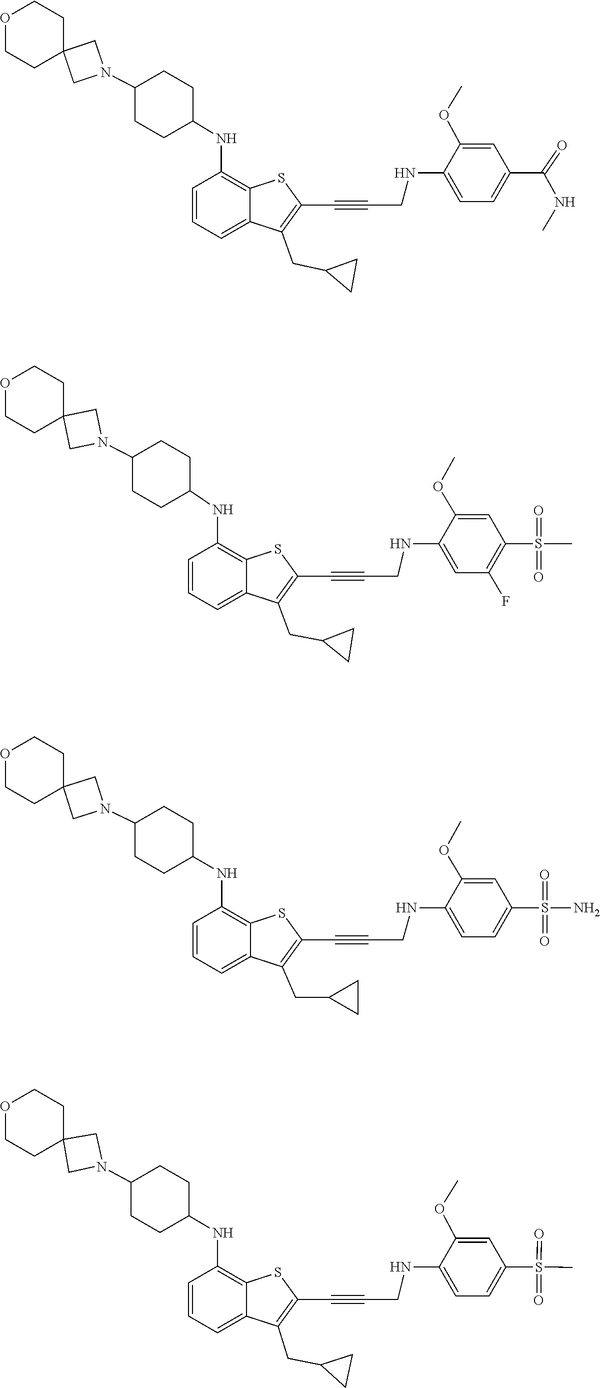 [SMILES: CNC(=O)c1ccc(NCC#Cc2sc3c(NC4CCC(N5CC6(CCOCC6)C5)CC4)cccc3c2CC2CC2)c(OC)c1 ; COc1cc(S(C)(=O)=O)c(F)cc1NCC#Cc1sc2c(NC3CCC(N4CC5(CCOCC5)C4)CC3)cccc2c1CC1CC1 ; COc1cc(S(N)(=O)=O)ccc1NCC#Cc1sc2c(NC3CCC(N4CC5(CCOCC5)C4)CC3)cccc2c1CC1CC1 ; COc1cc(S(C)(=O)=O)ccc1NCC#Cc1sc2c(NC3CCC(N4CC5(CCOCC5)C4)CC3)cccc2c1CC1CC1] 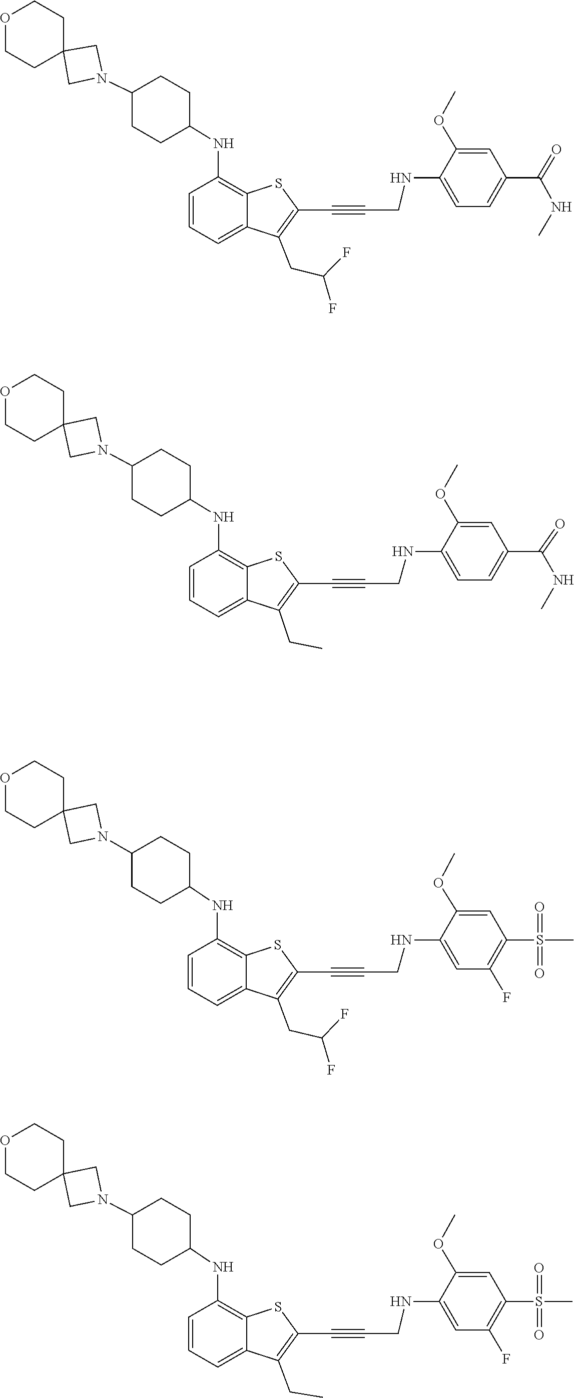 [SMILES: CNC(=O)c1ccc(NCC#Cc2sc3c(NC4CCC(N5CC6(CCOCC6)C5)CC4)cccc3c2CC(F)F)c(OC)c1 ; CCc1c(C#CCNc2ccc(C(=O)NC)cc2OC)sc2c(NC3CCC(N4CC5(CCOCC5)C4)CC3)cccc12 ; COc1cc(S(C)(=O)=O)c(F)cc1NCC#Cc1sc2c(NC3CCC(N4CC5(CCOCC5)C4)CC3)cccc2c1CC(F)F ; CCc1c(C#CCNc2cc(F)c(S(C)(=O)=O)cc2OC)sc2c(NC3CCC(N4CC5(CCOCC5)C4)CC3)cccc12] 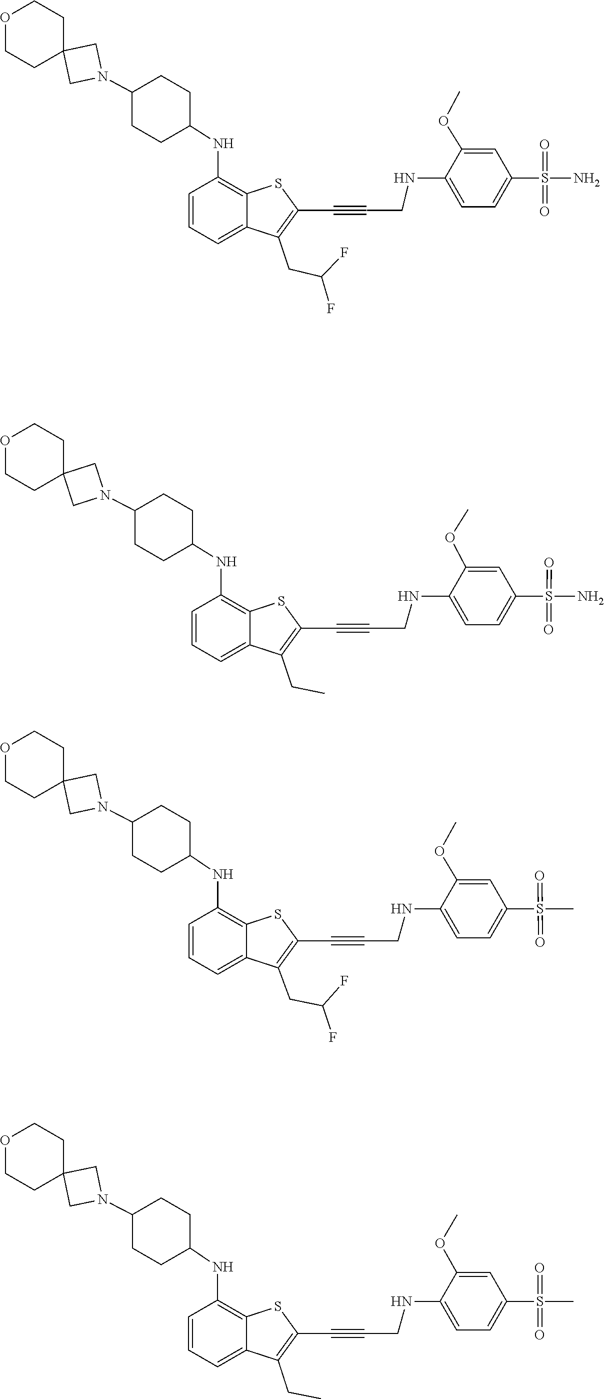 [SMILES: COc1cc(S(N)(=O)=O)ccc1NCC#Cc1sc2c(NC3CCC(N4CC5(CCOCC5)C4)CC3)cccc2c1CC(F)F ; CCc1c(C#CCNc2ccc(S(N)(=O)=O)cc2OC)sc2c(NC3CCC(N4CC5(CCOCC5)C4)CC3)cccc12 ; COc1cc(S(C)(=O)=O)ccc1NCC#Cc1sc2c(NC3CCC(N4CC5(CCOCC5)C4)CC3)cccc2c1CC(F)F ; CCc1c(C#CCNc2ccc(S(C)(=O)=O)cc2OC)sc2c(NC3CCC(N4CC5(CCOCC5)C4)CC3)cccc12] 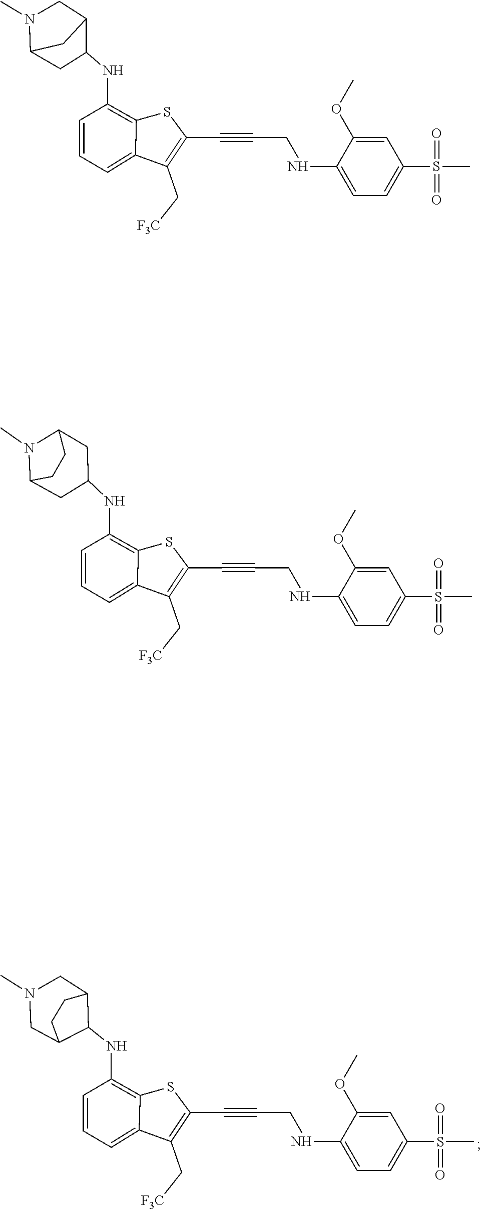
or the compound of formula (I) is selected from: 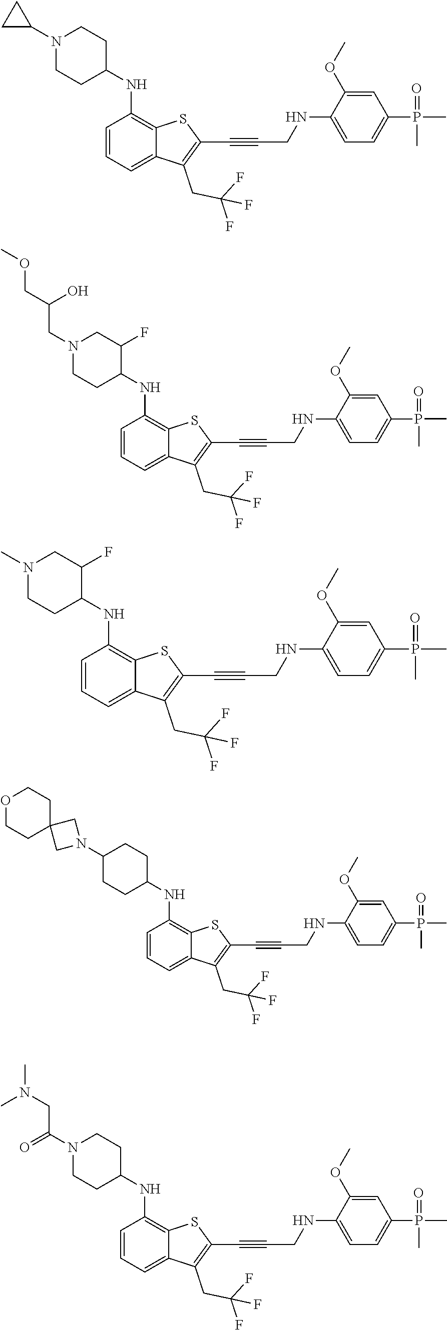 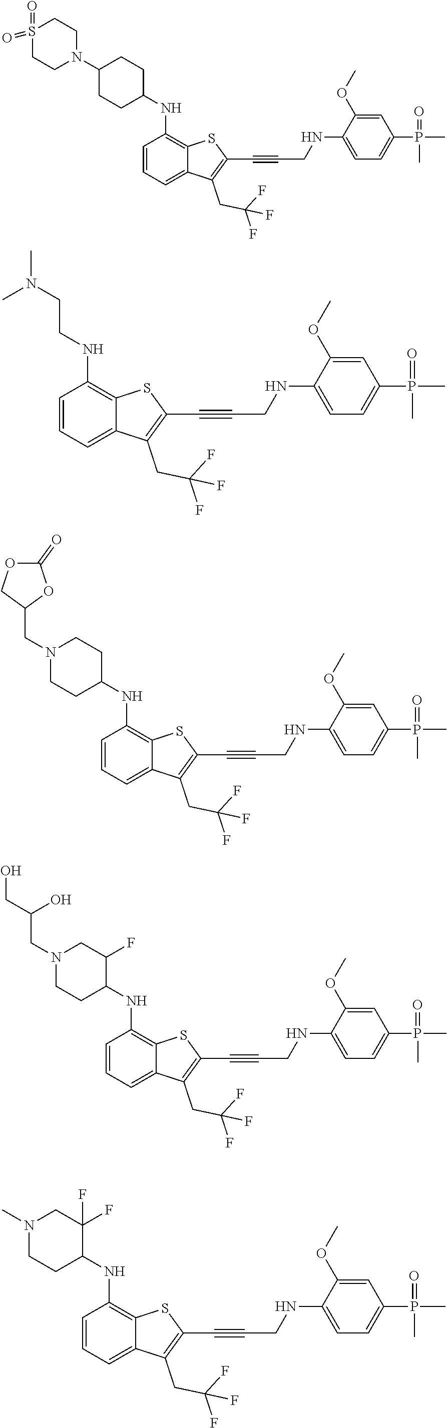 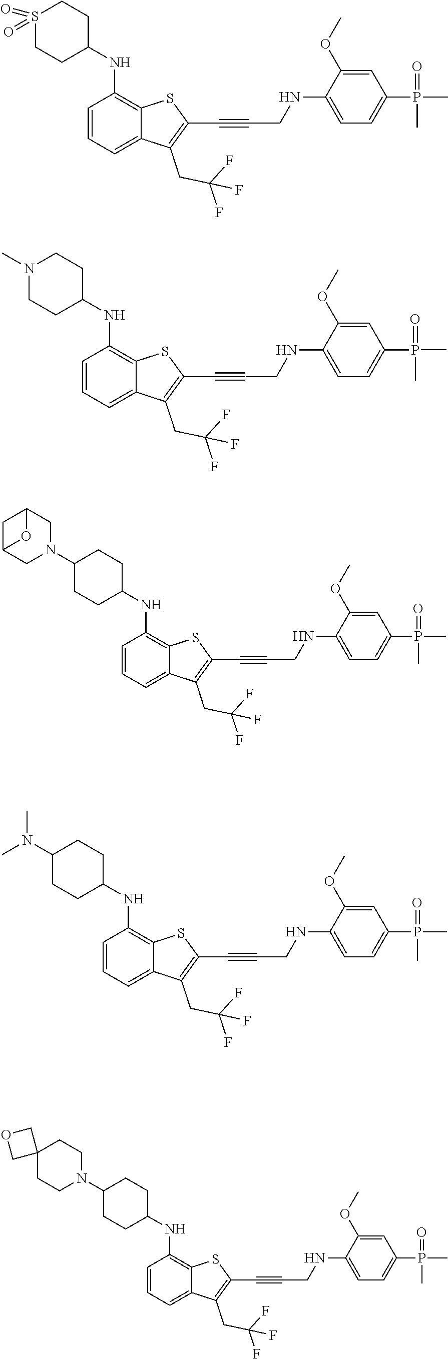 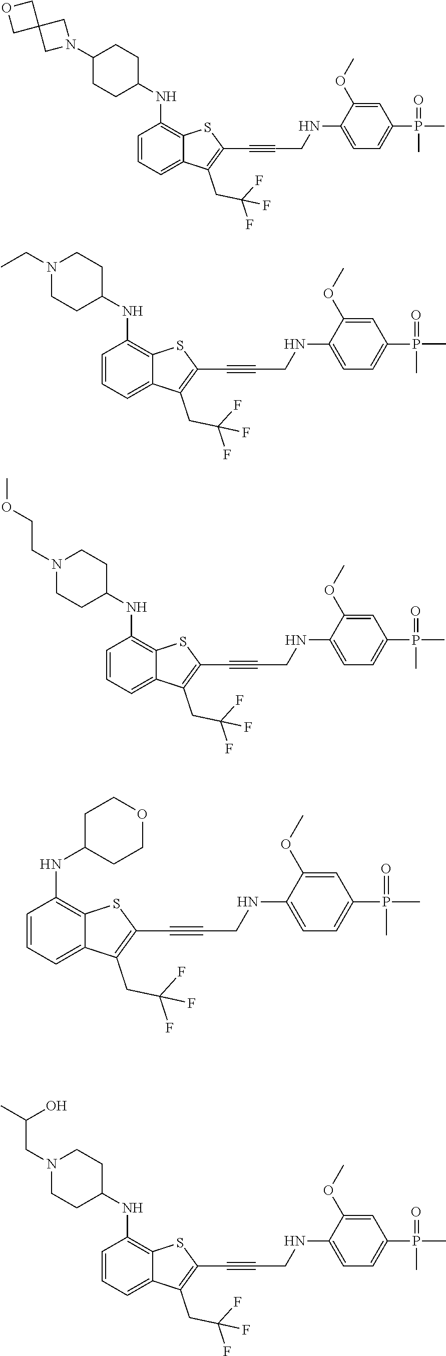 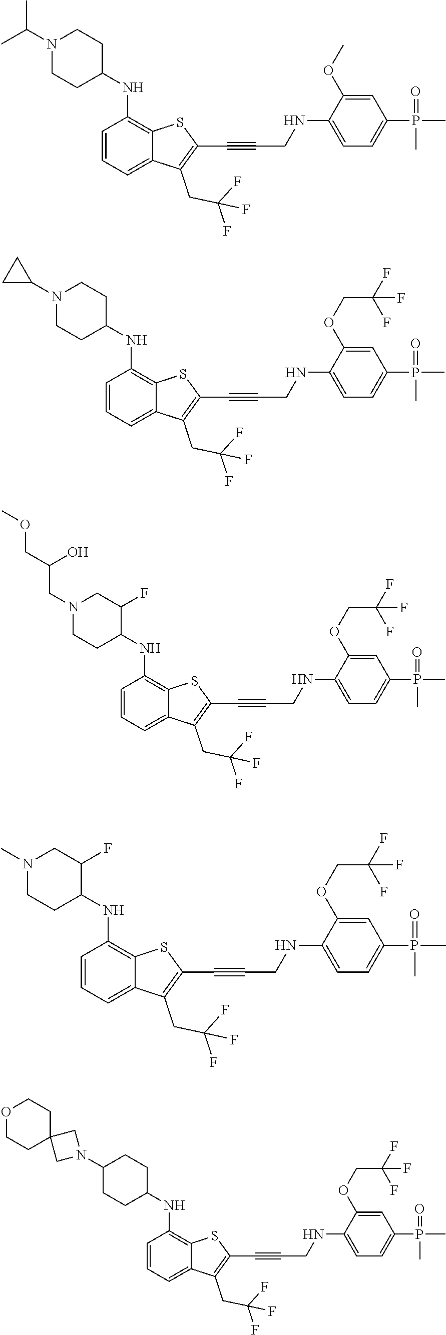 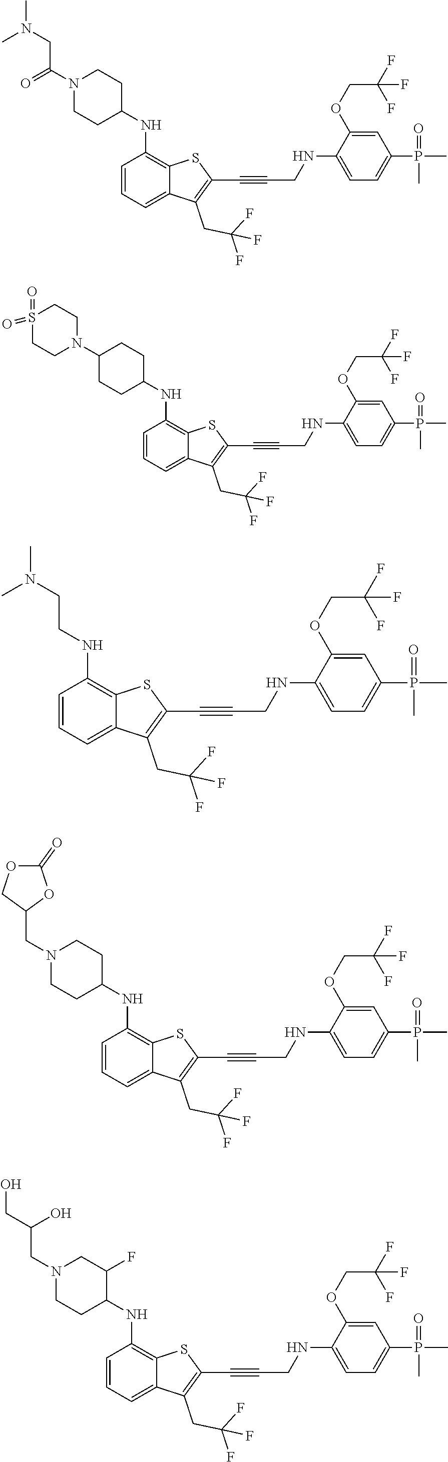 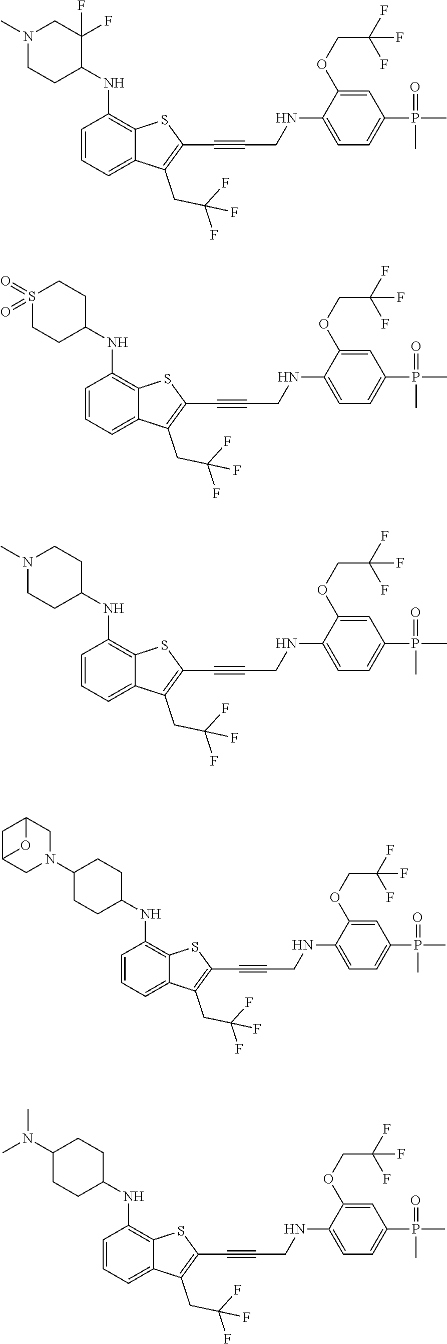 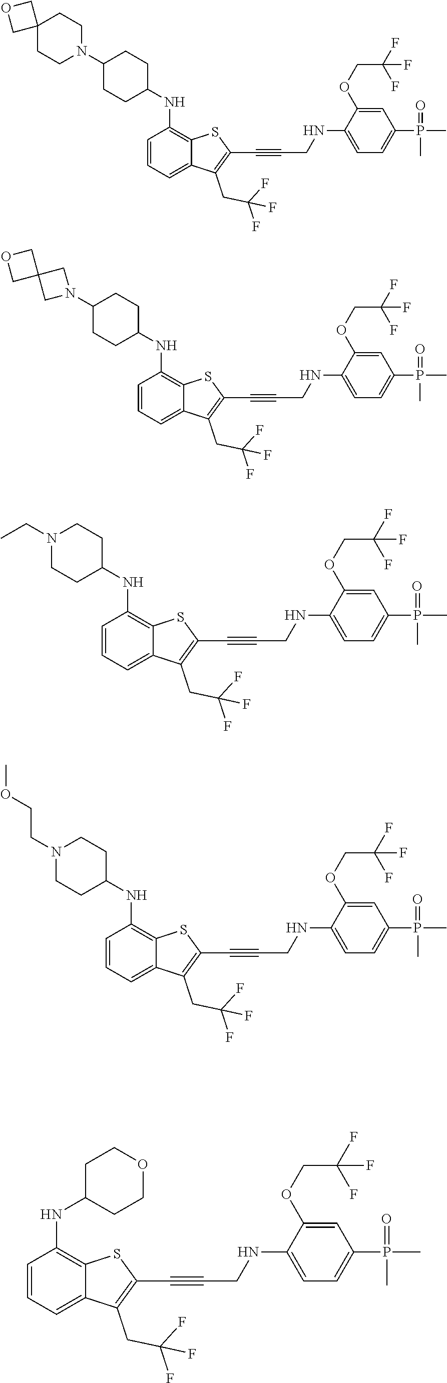 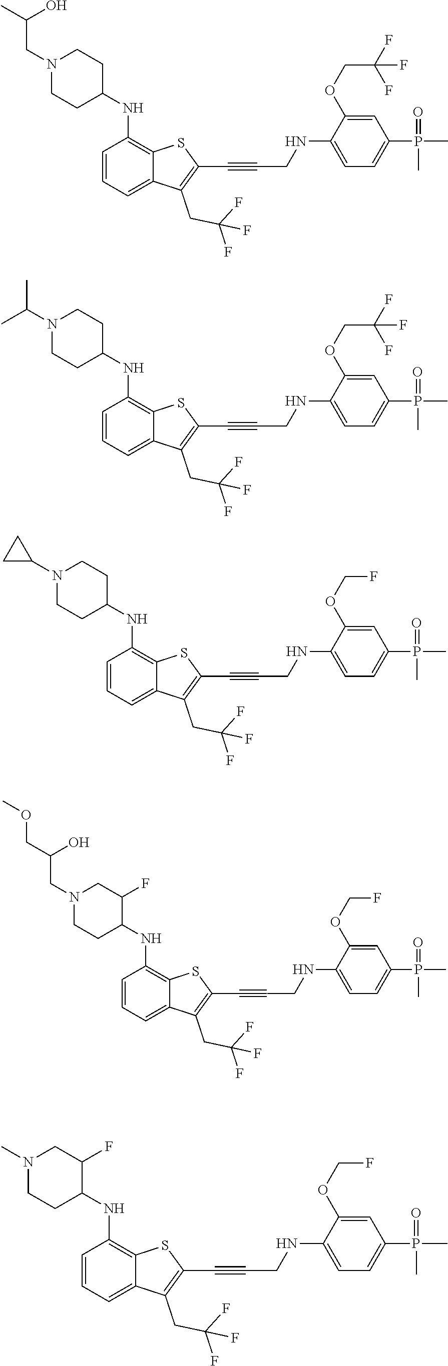 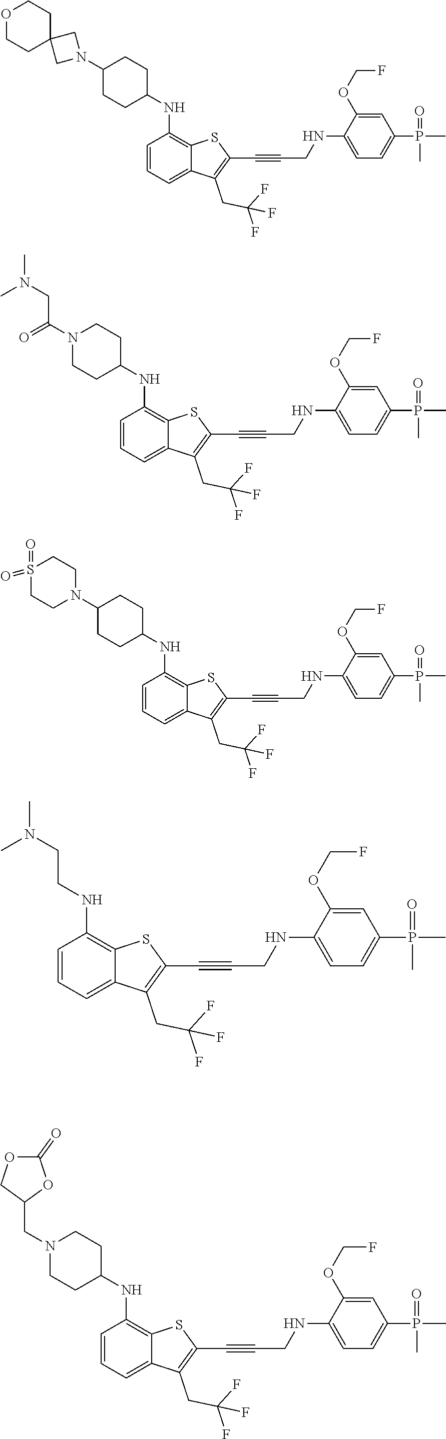 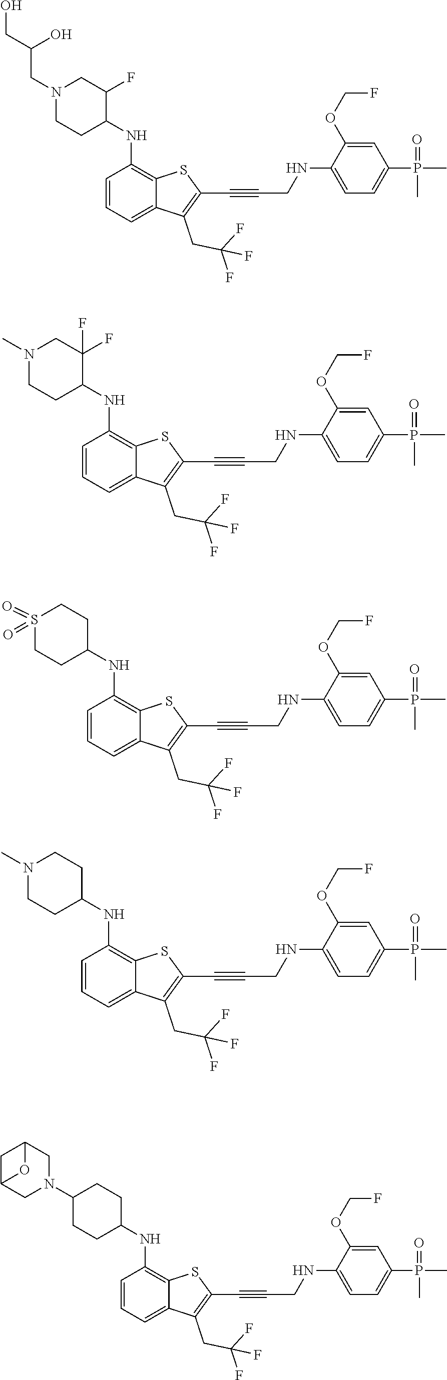 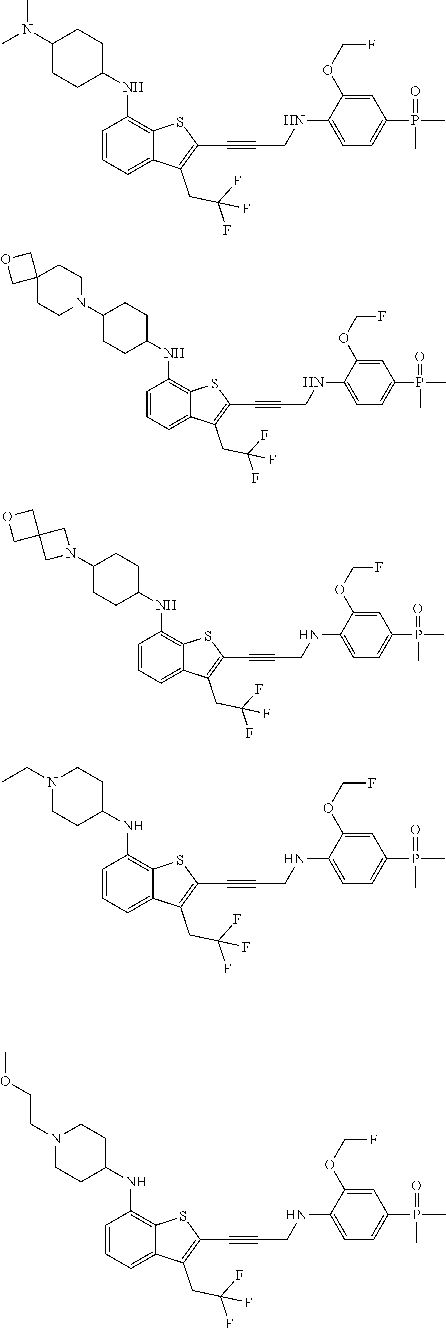 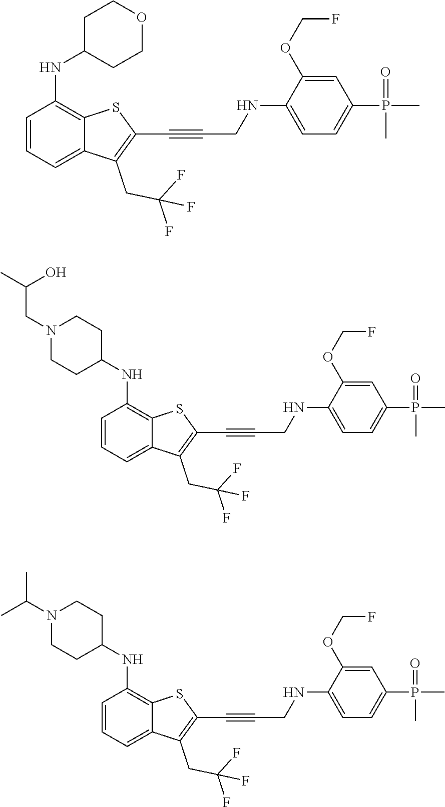 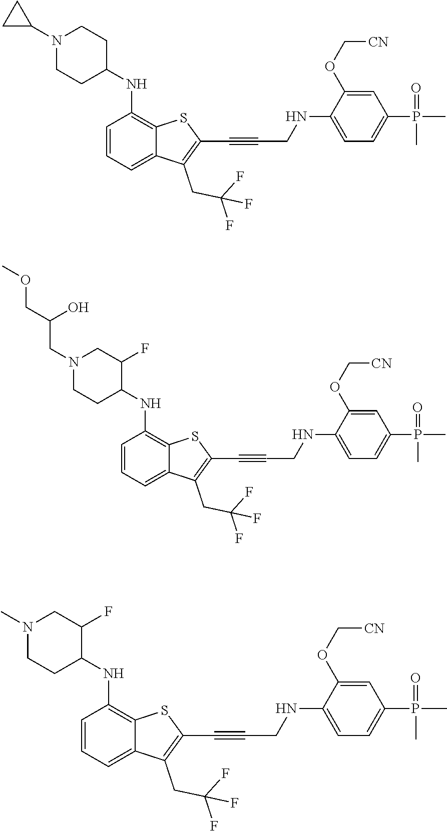 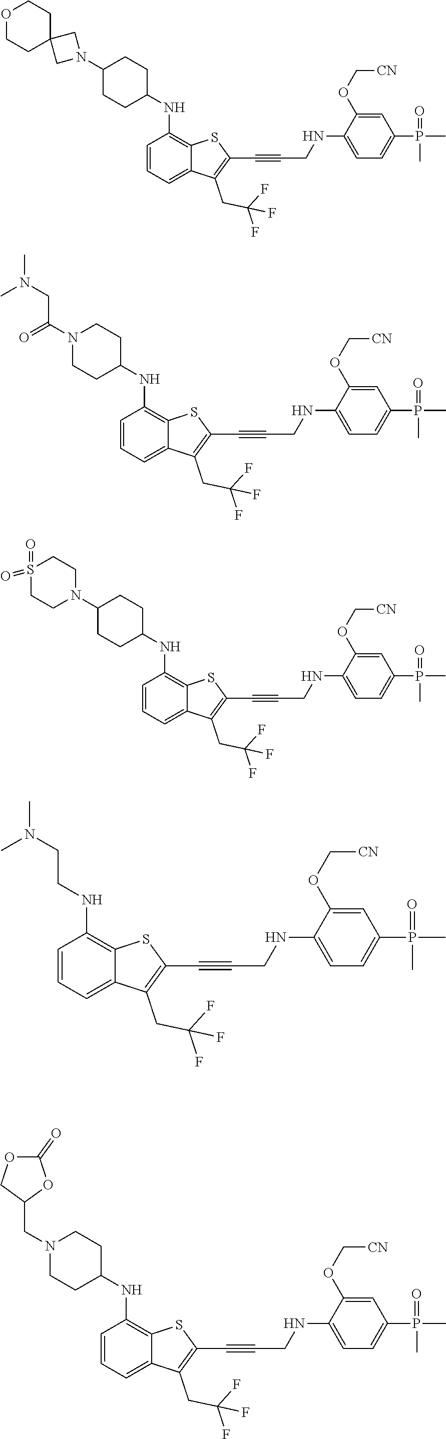 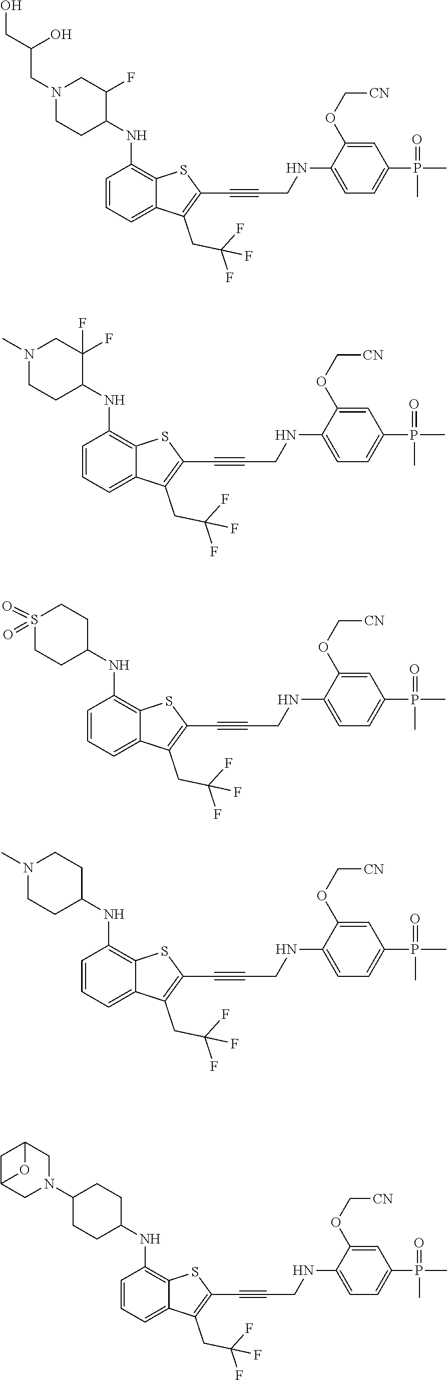 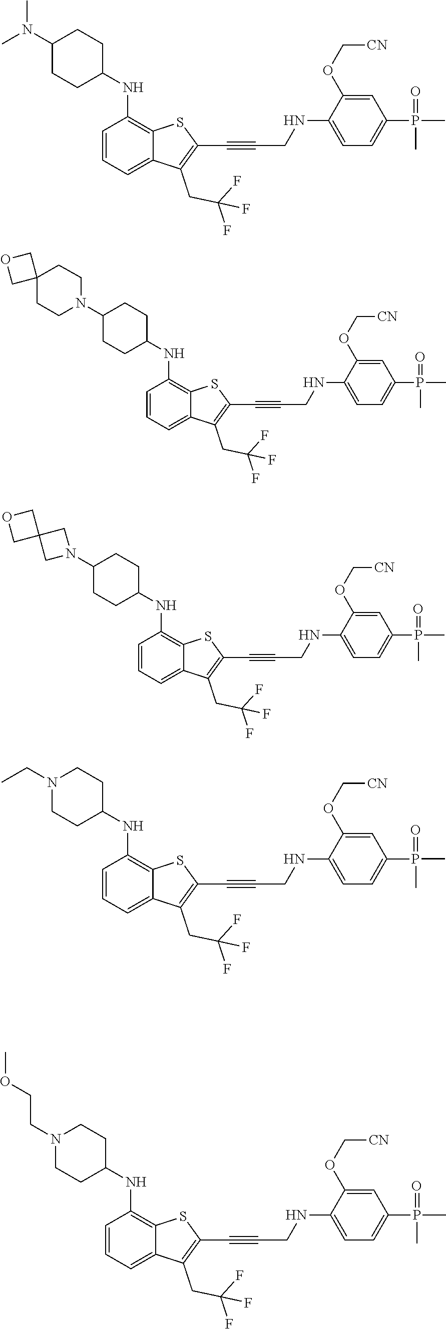 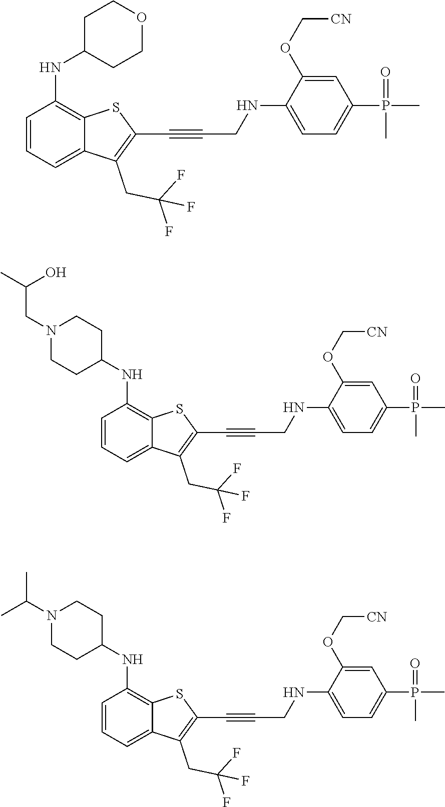 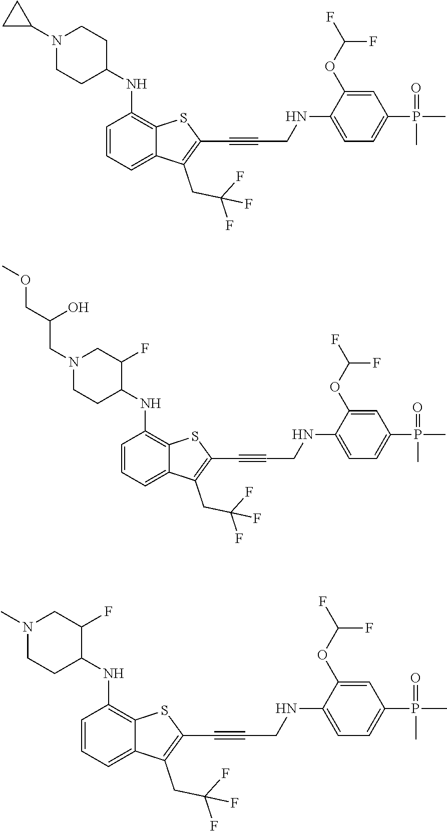 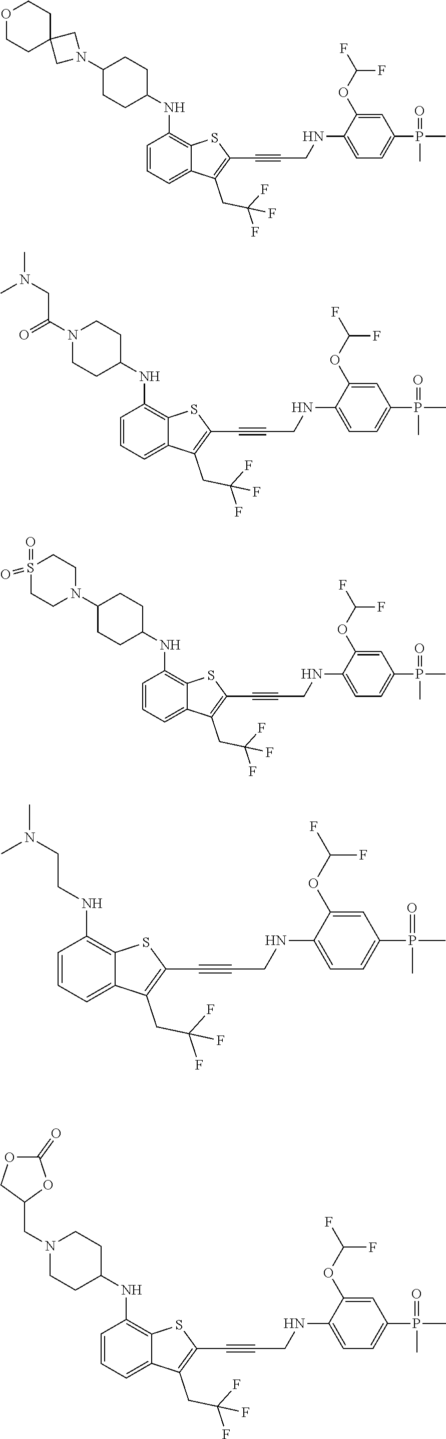 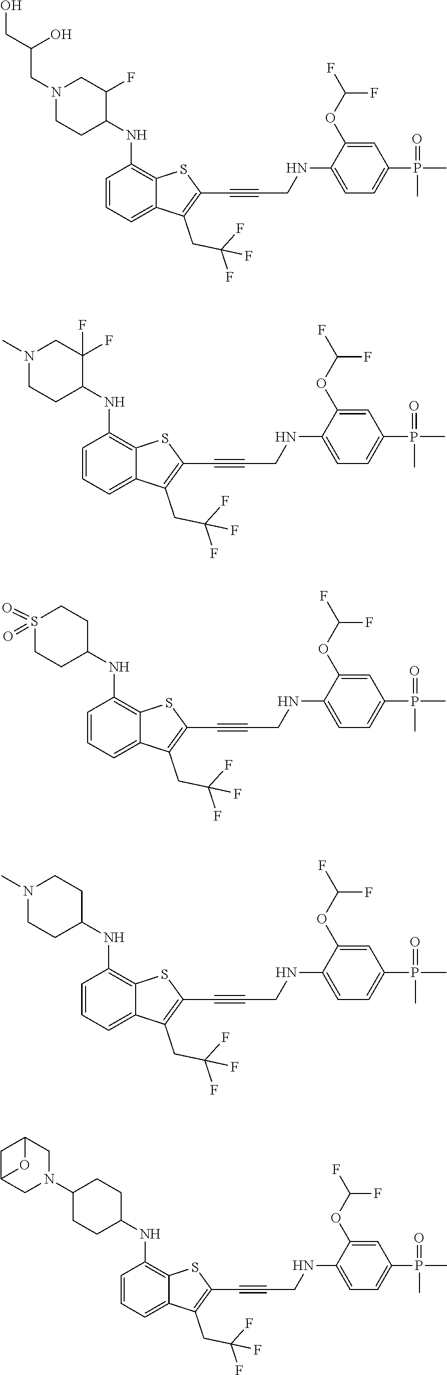 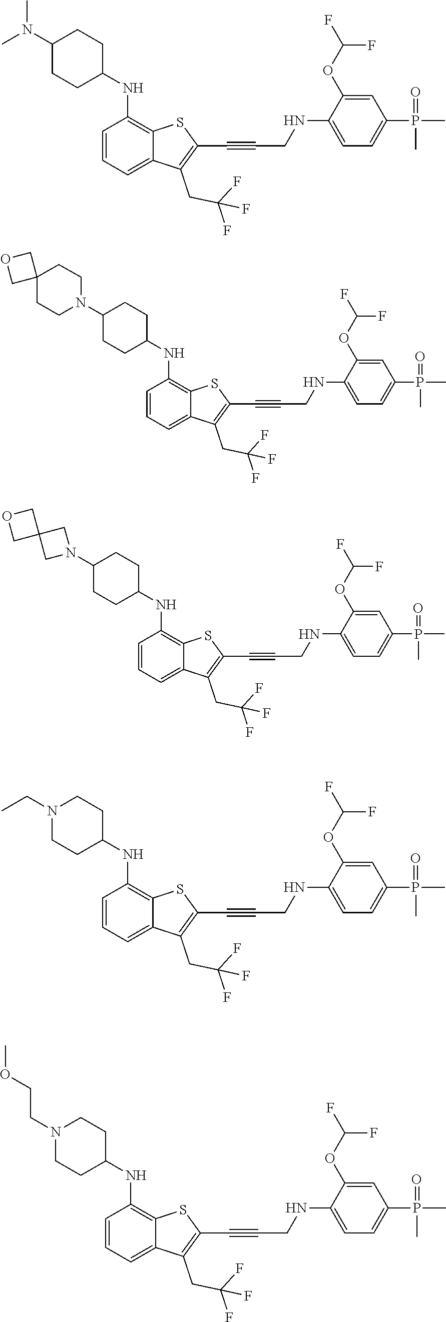 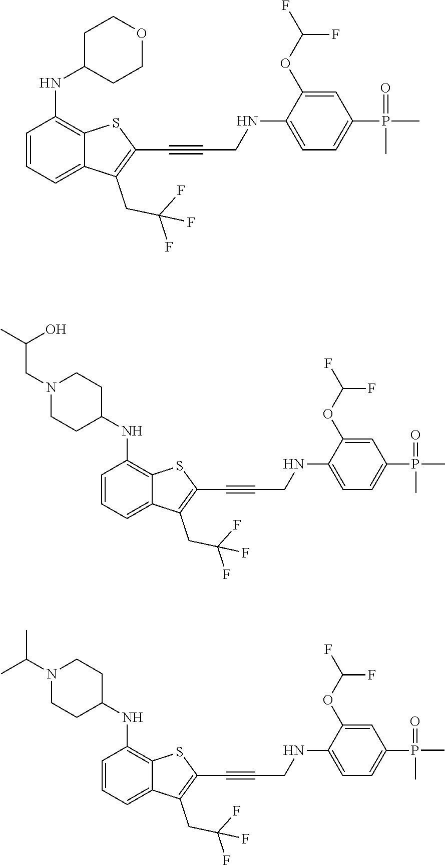 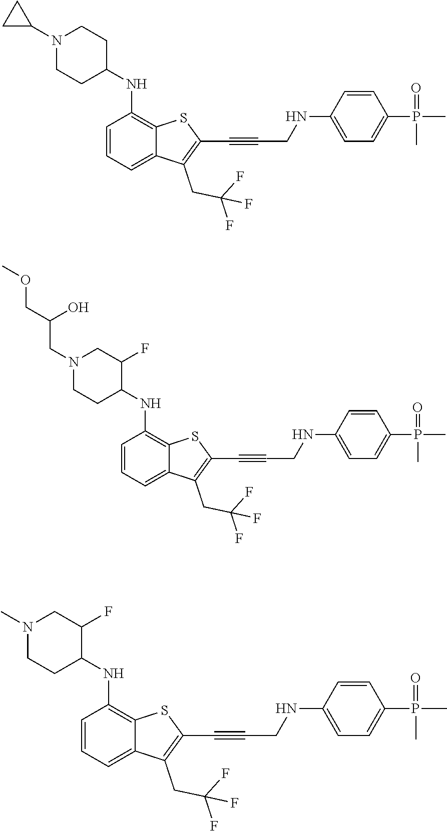 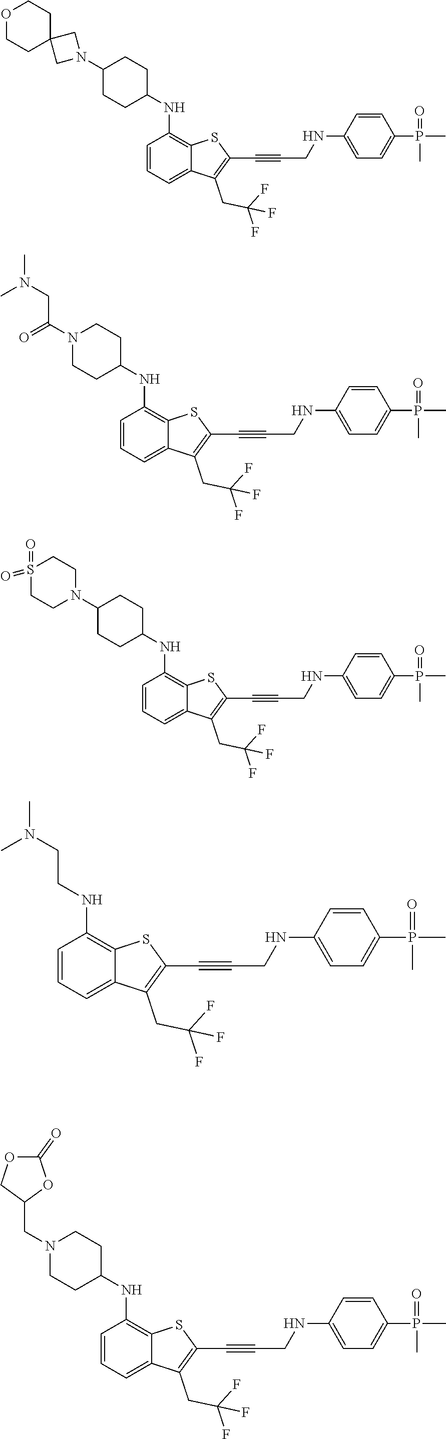 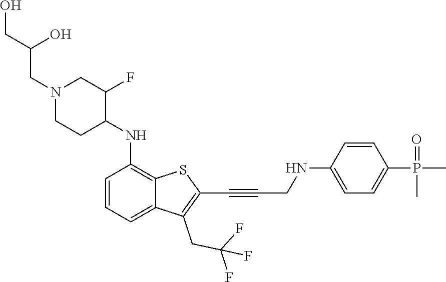 [SMILES: CP(C)(=O)c1ccc(NCC#Cc2sc3c(NC4CCN(CC(O)CO)CC4F)cccc3c2CC(F)(F)F)cc1] 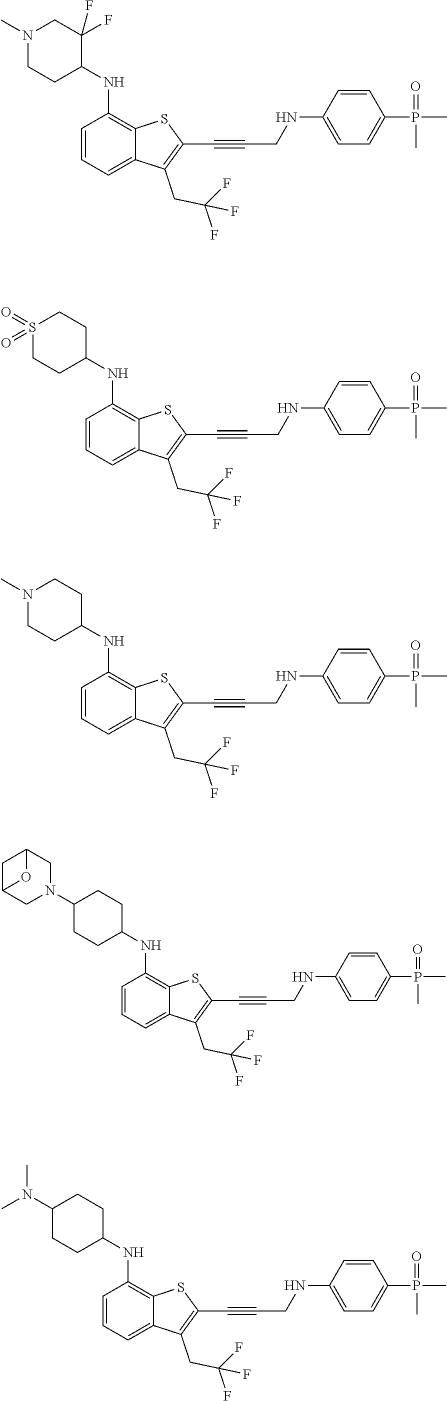 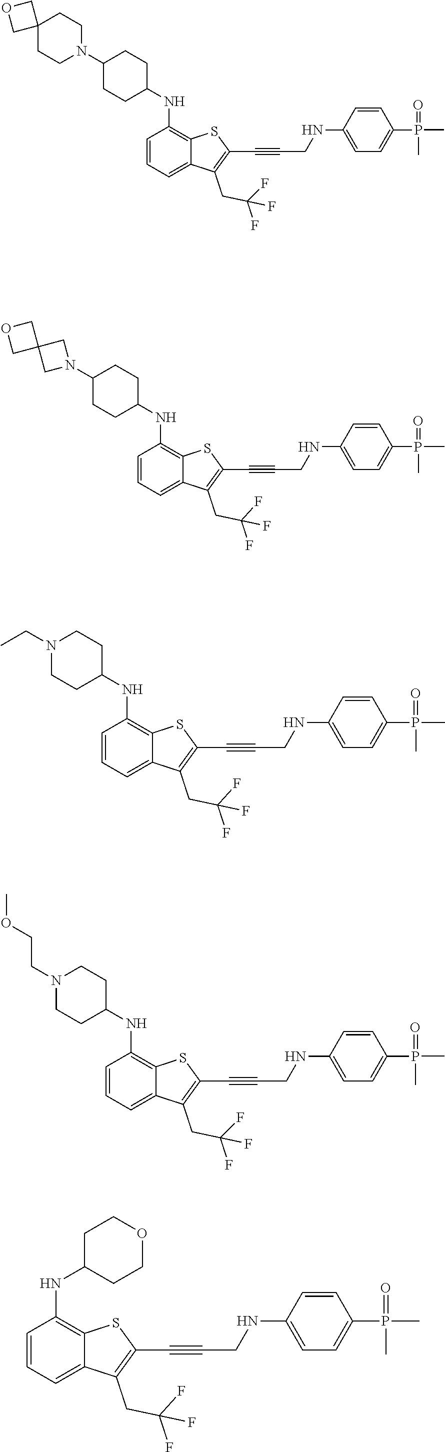 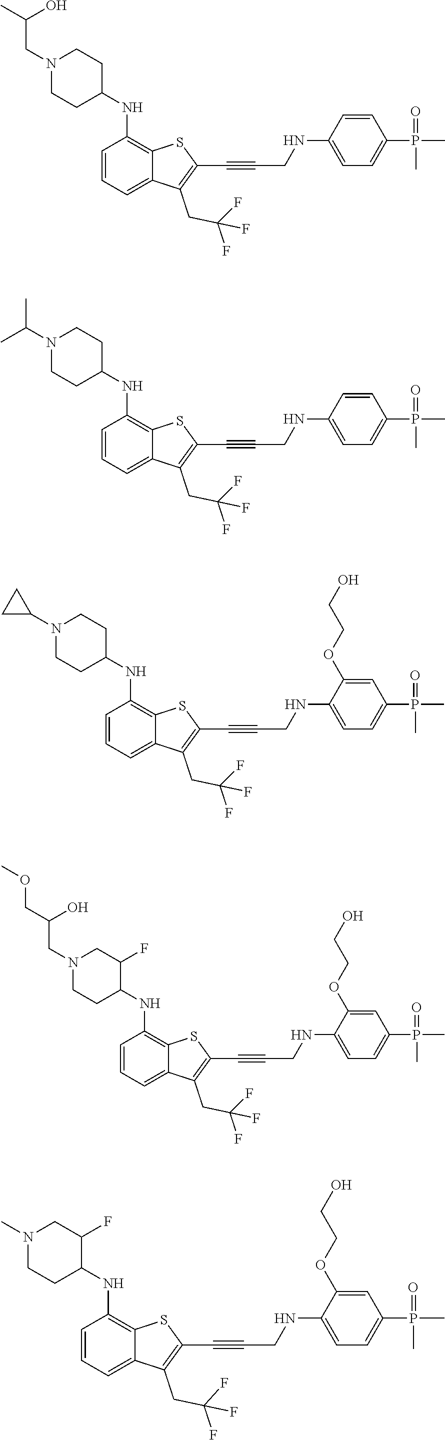 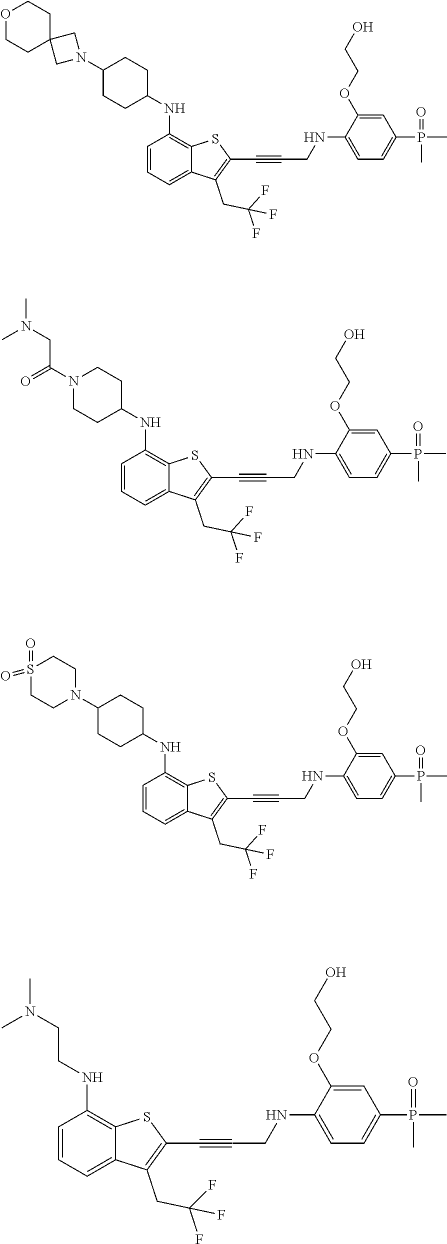 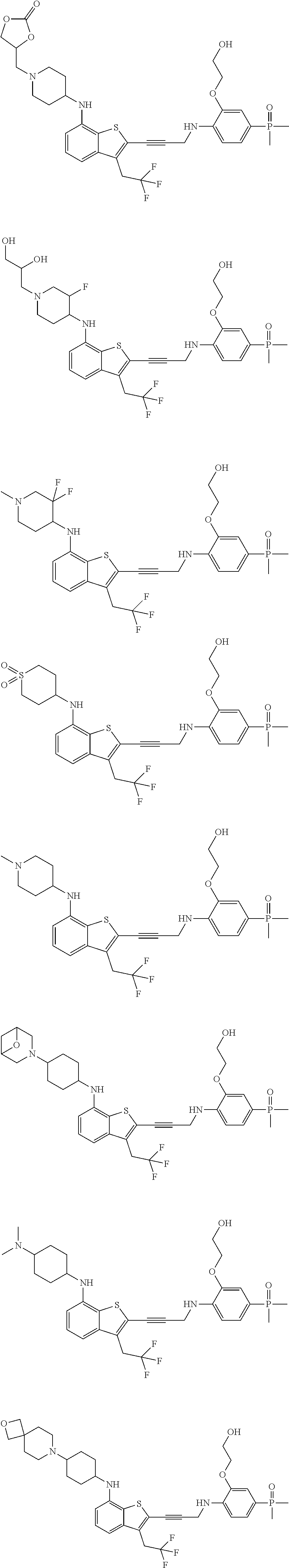 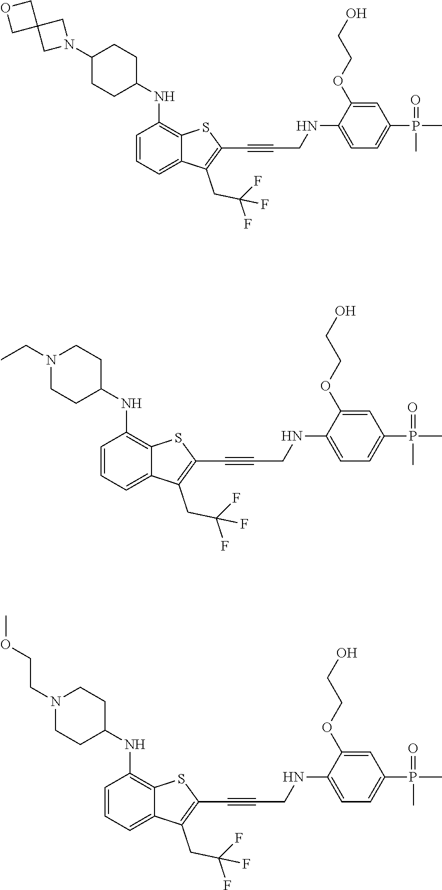 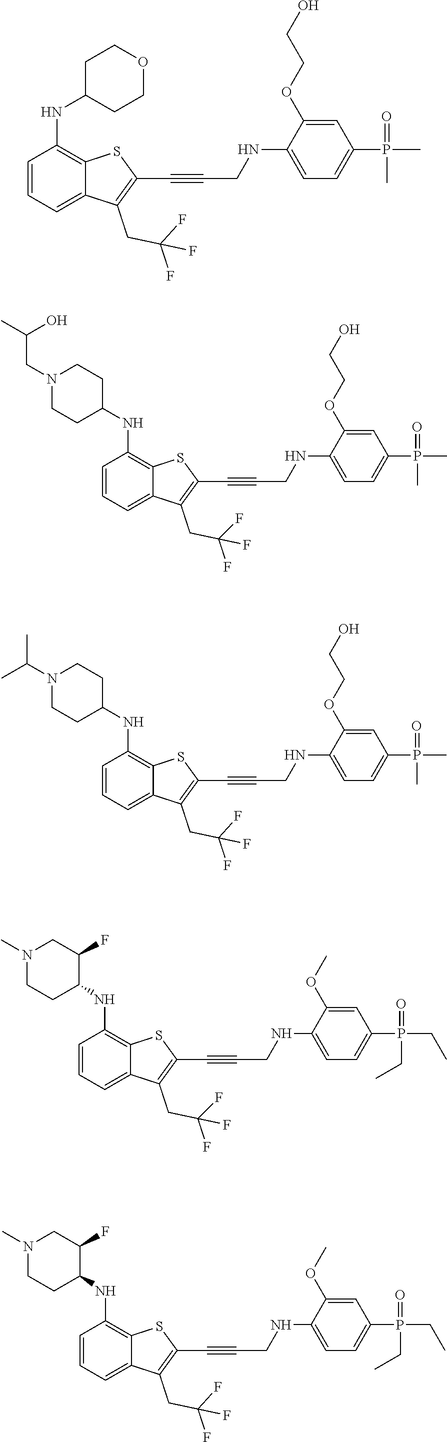 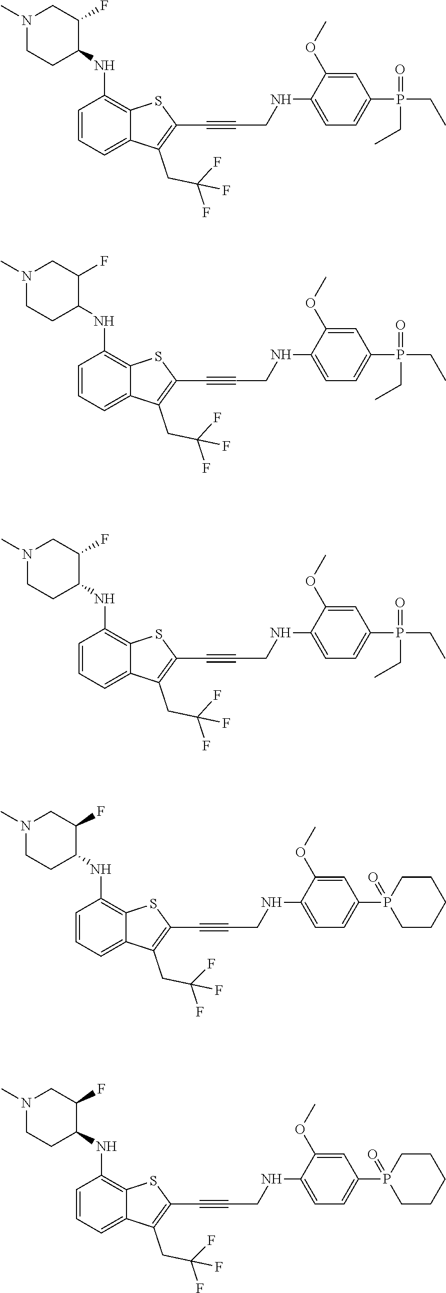 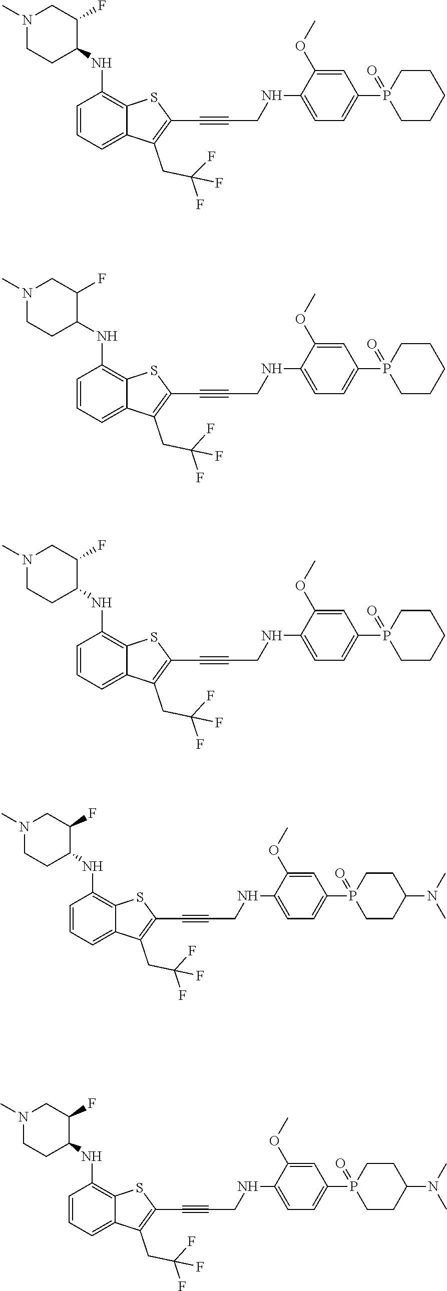 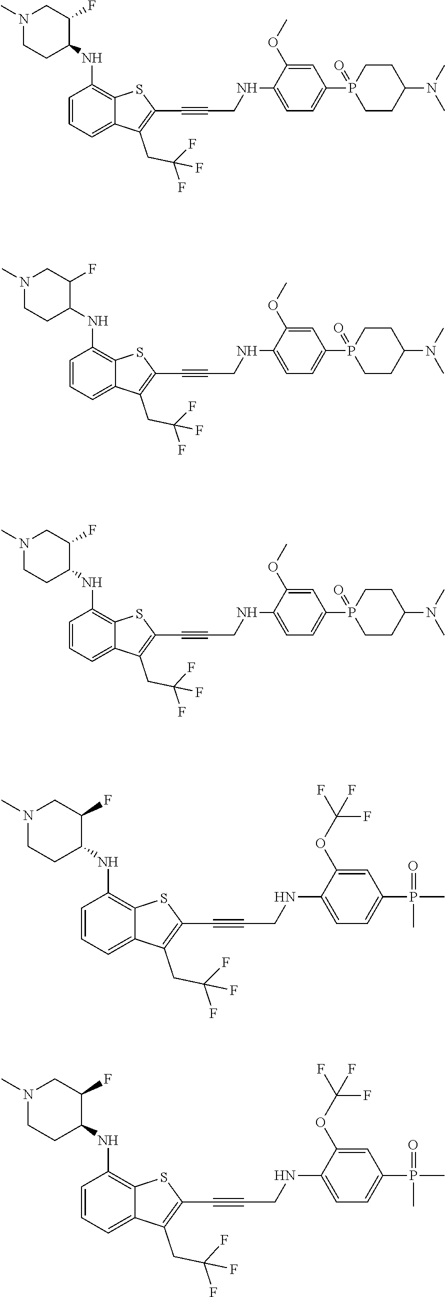 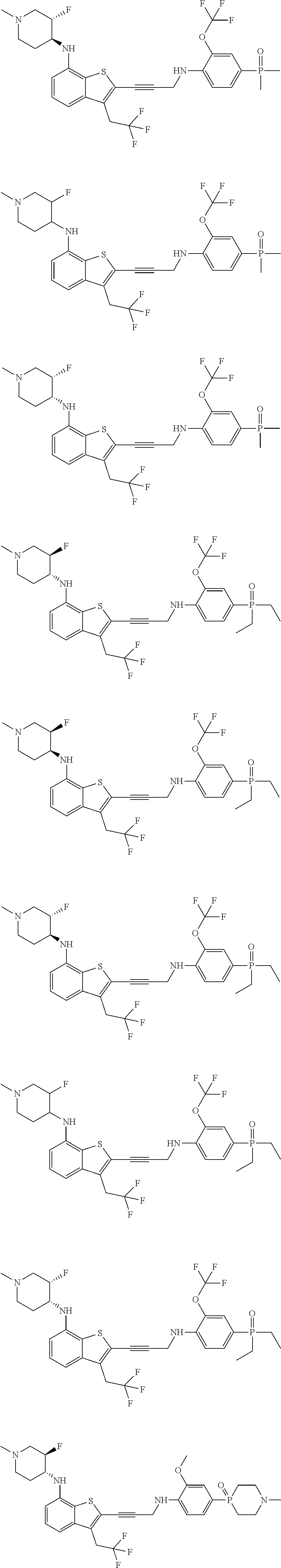 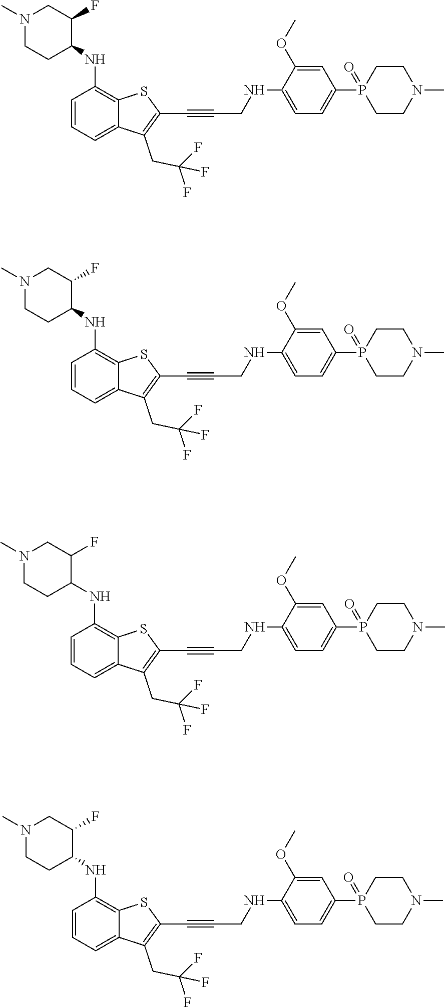 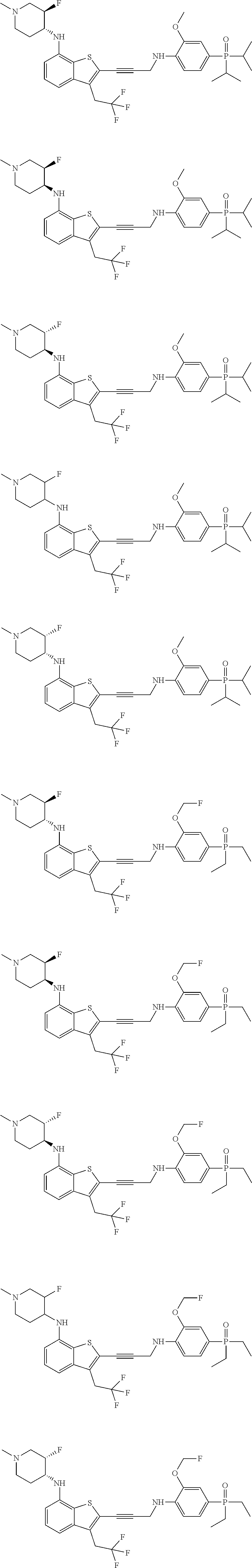 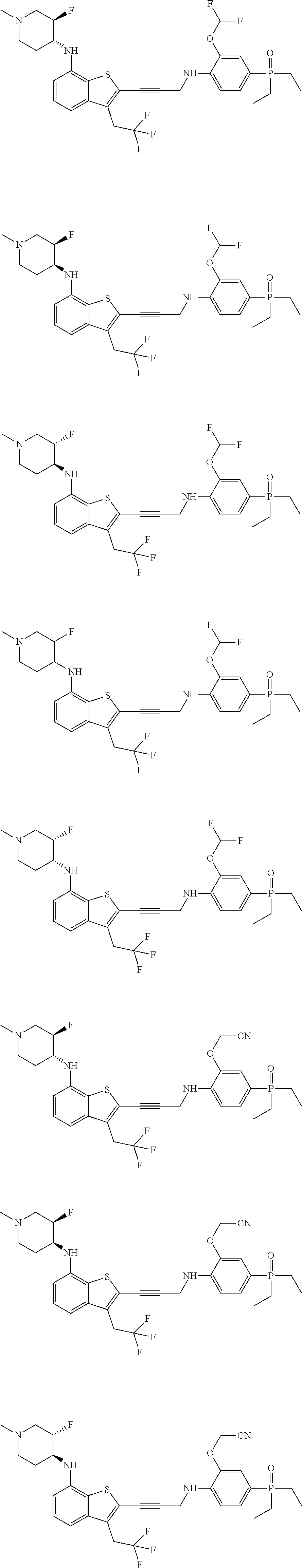 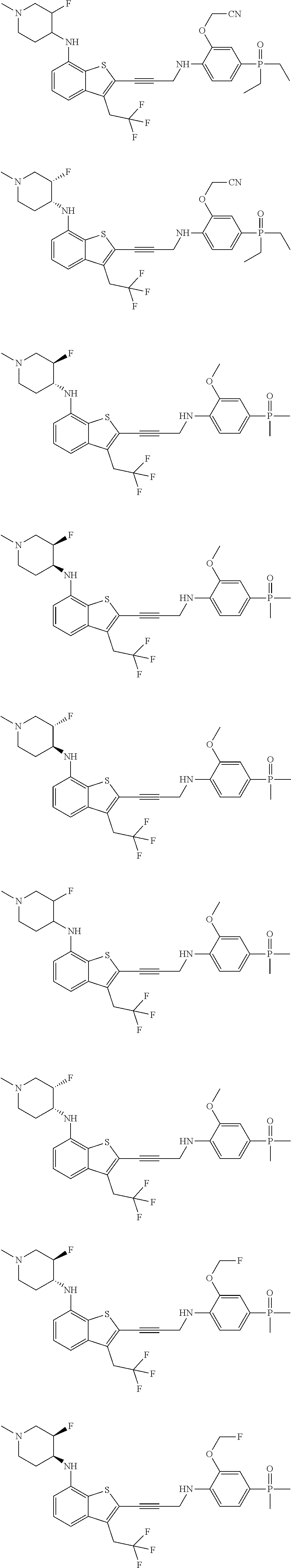 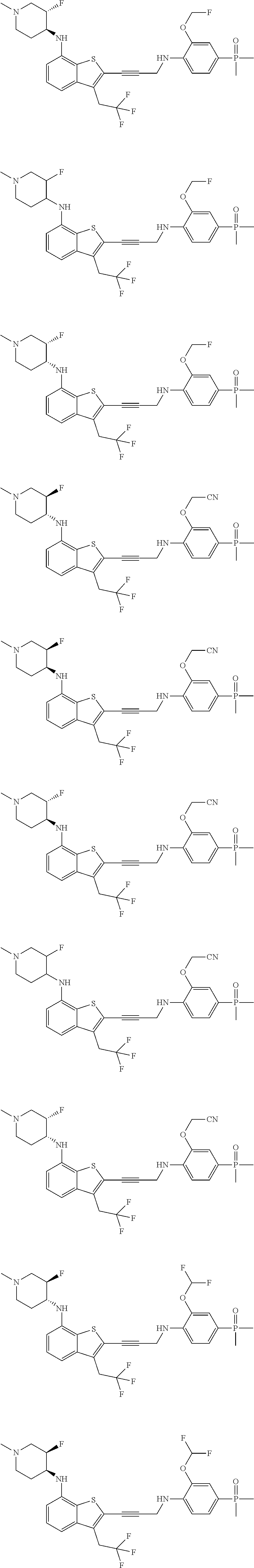 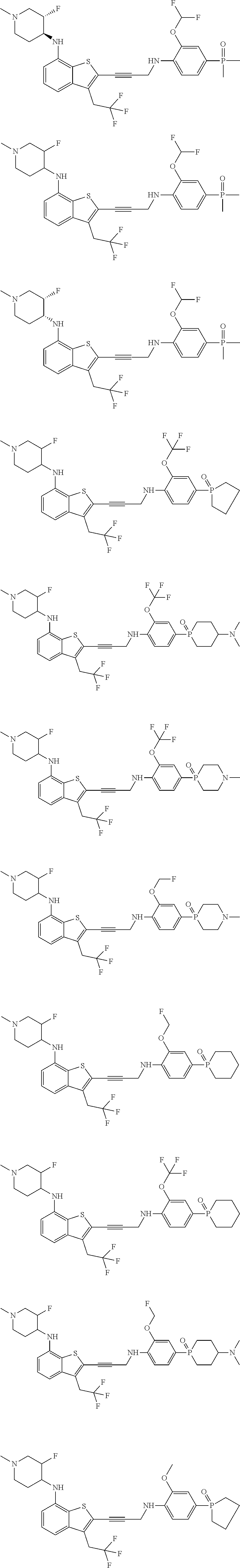 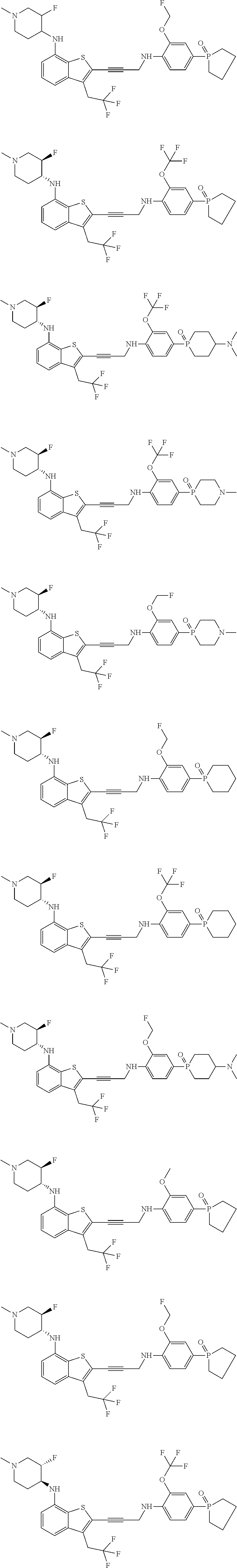 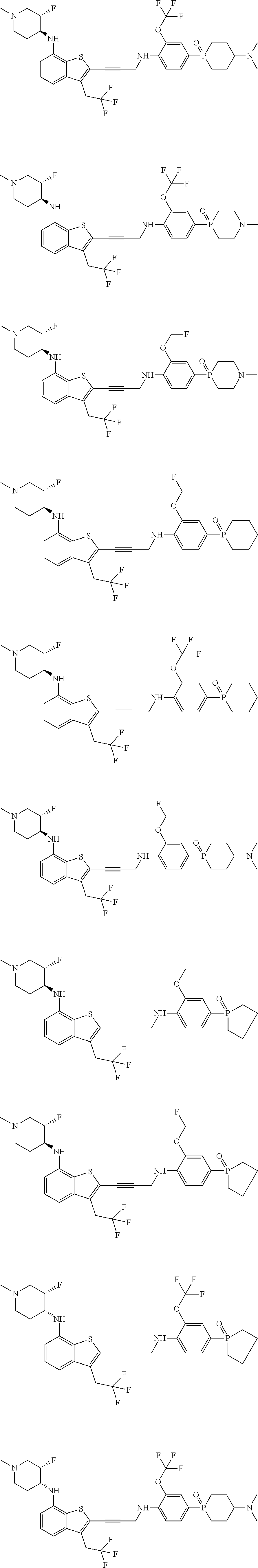 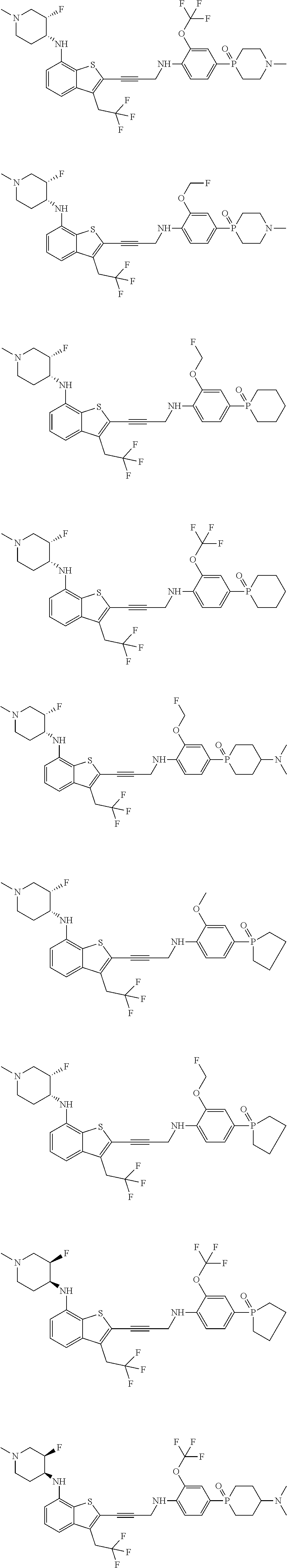 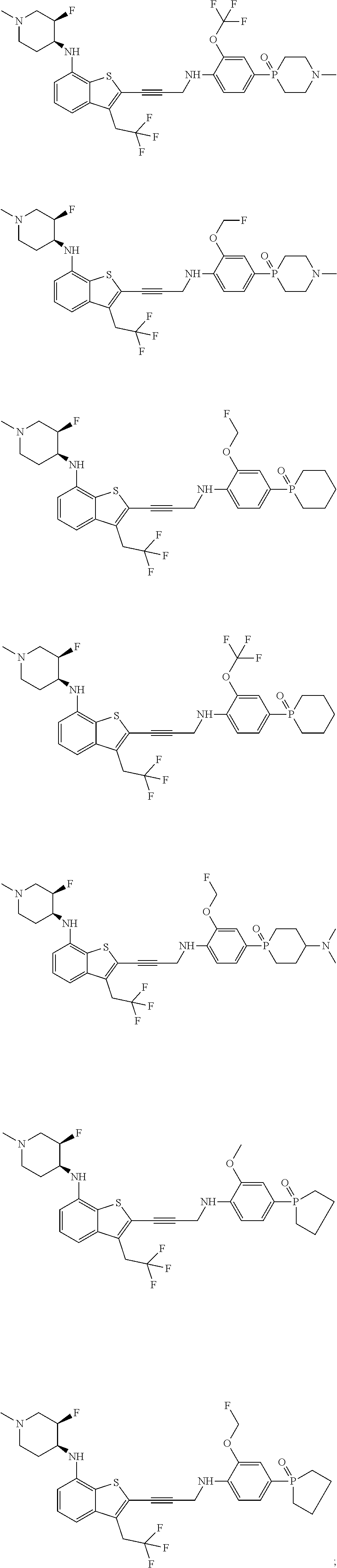
or the compound of formula (I) is selected from:

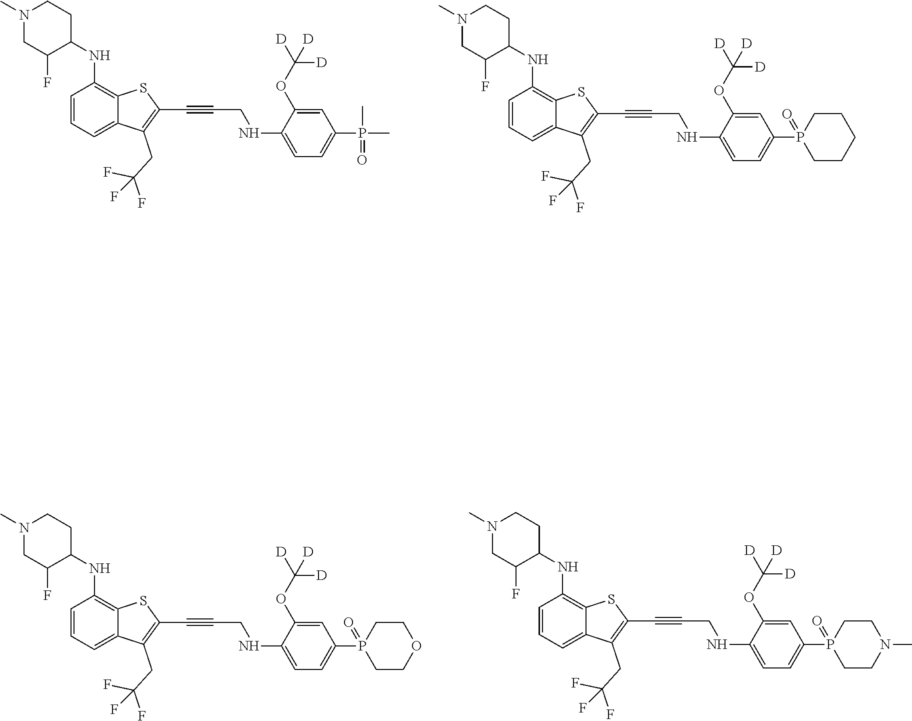

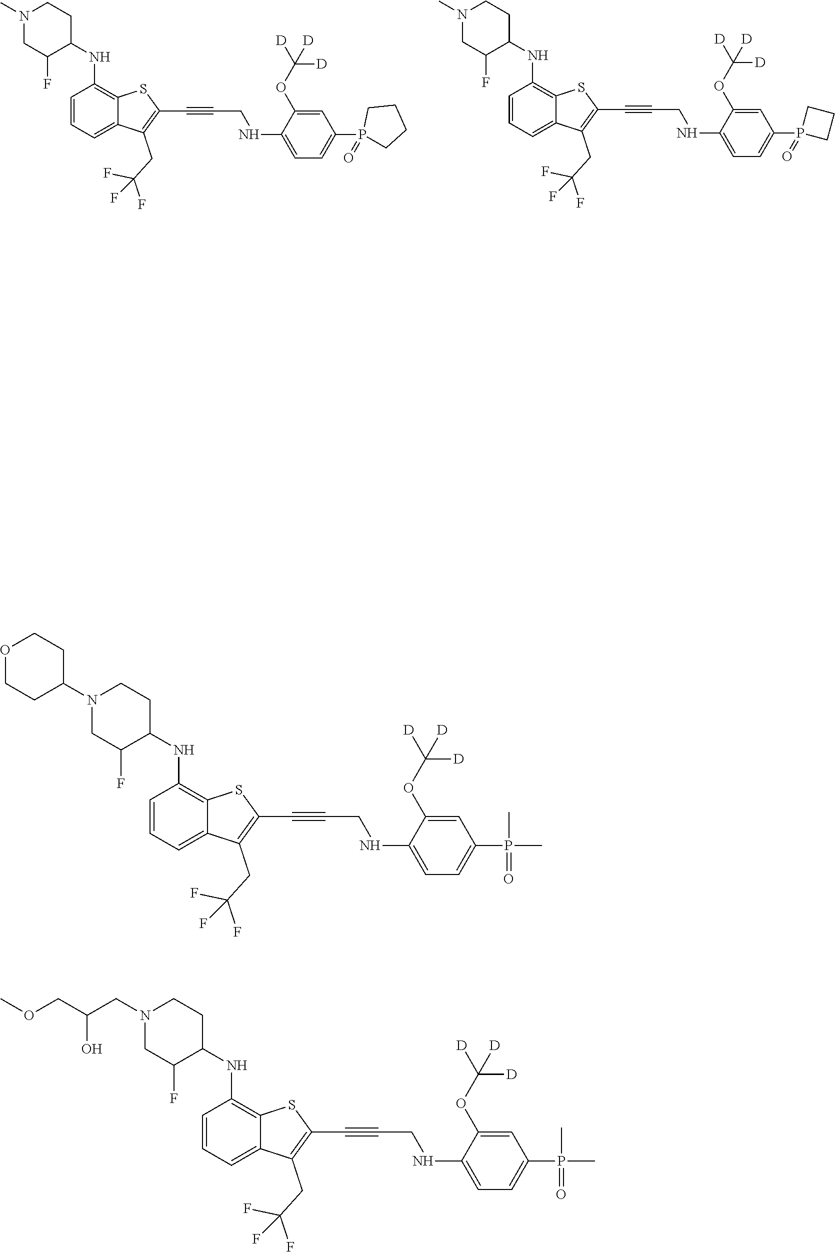

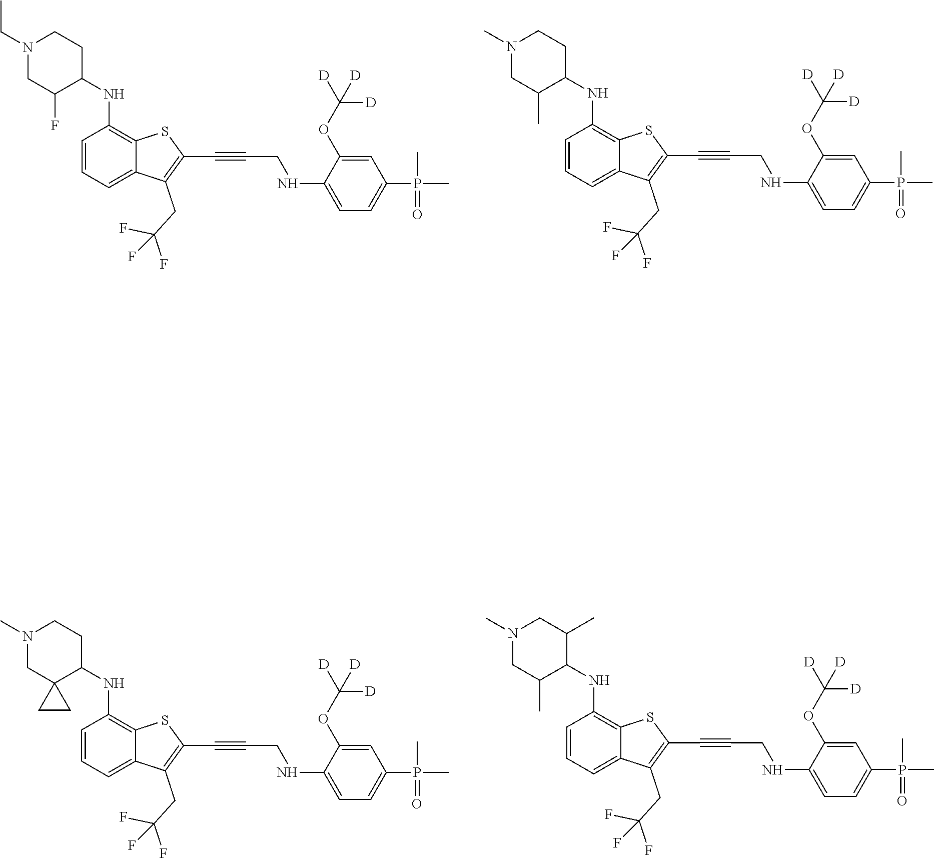

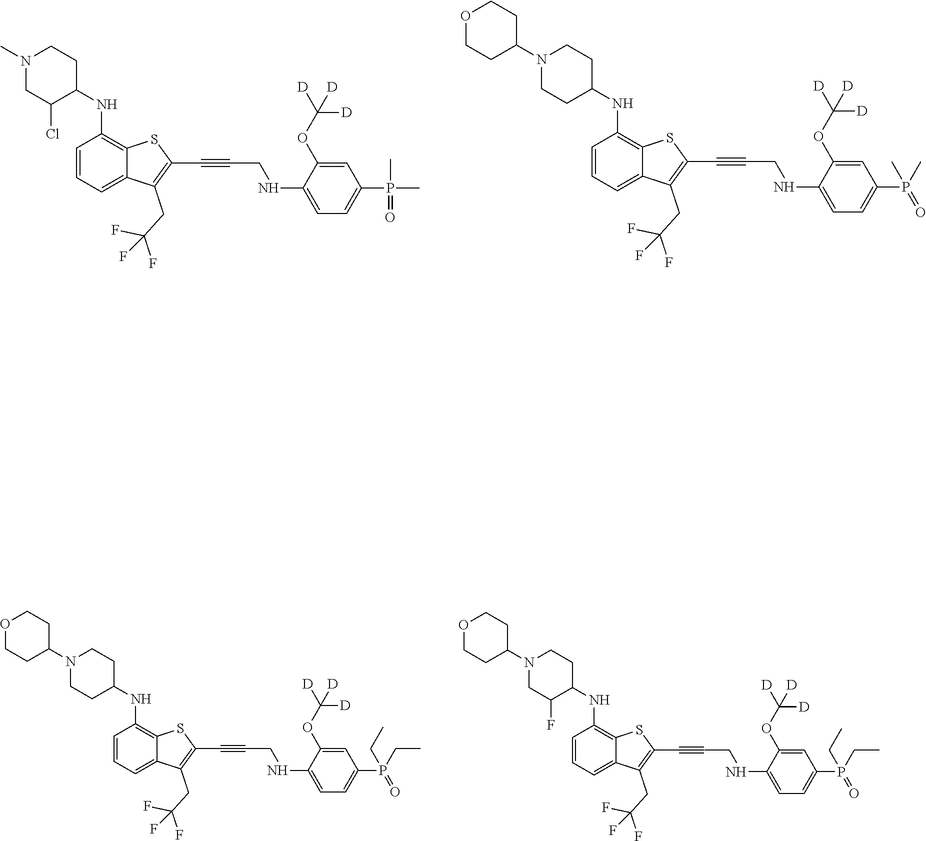

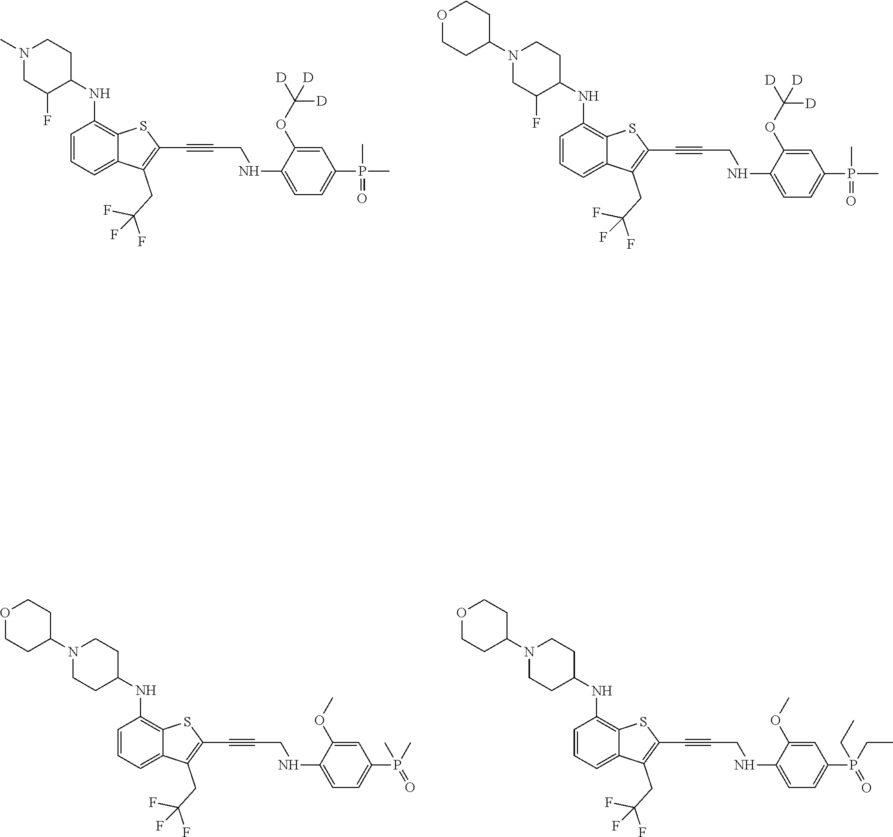

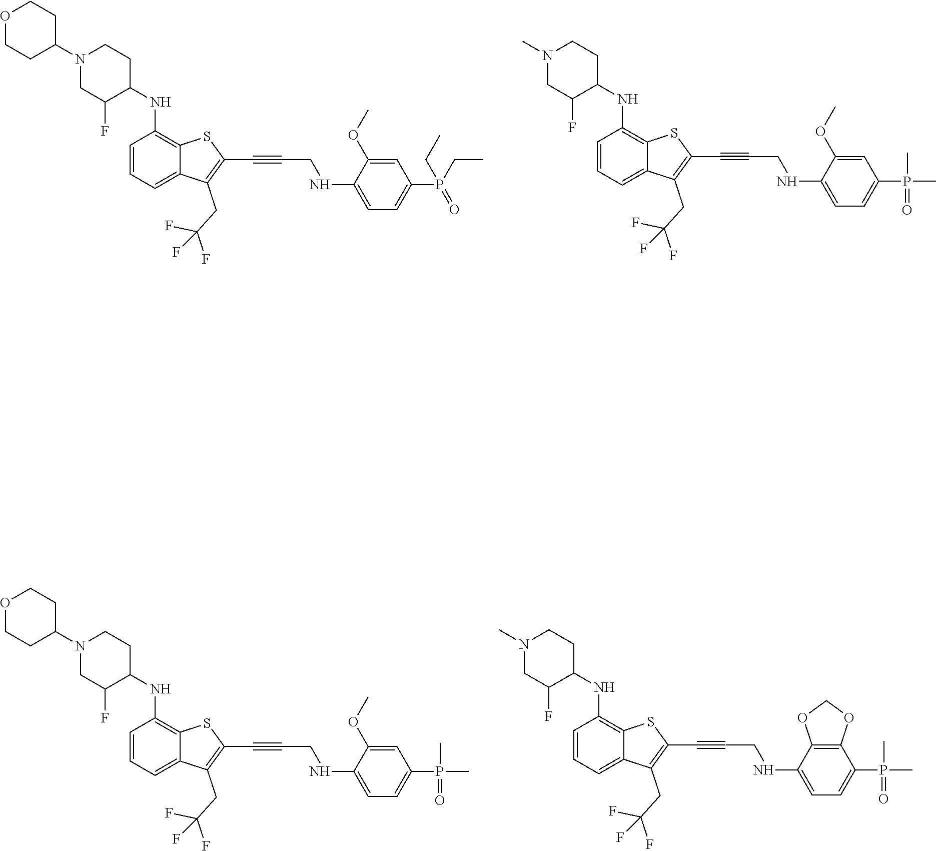

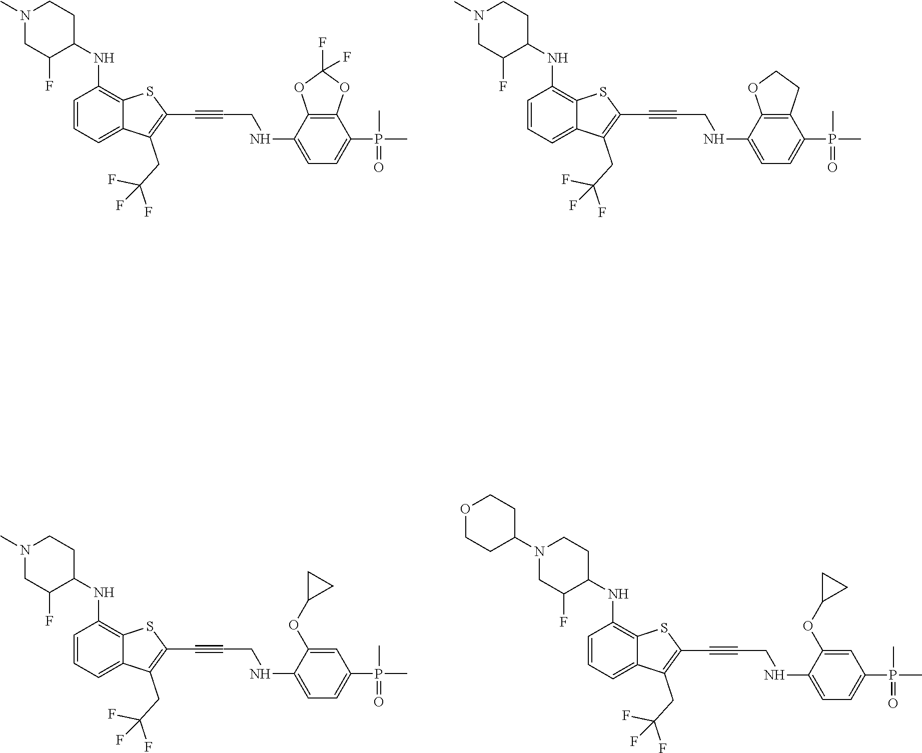

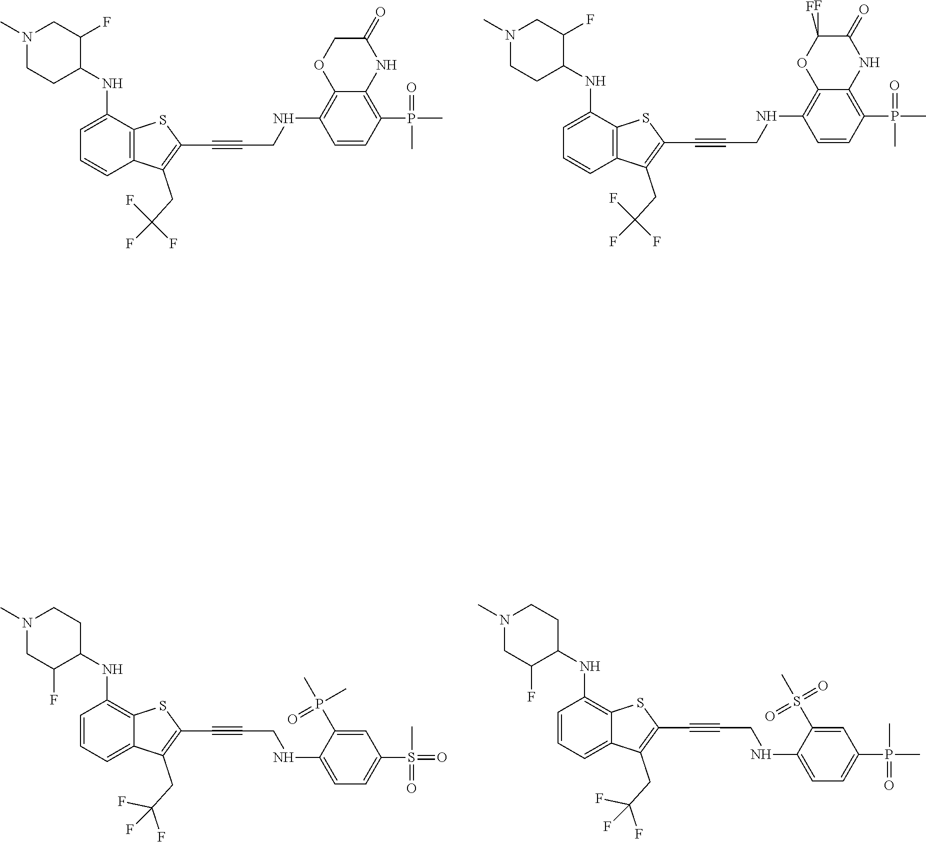

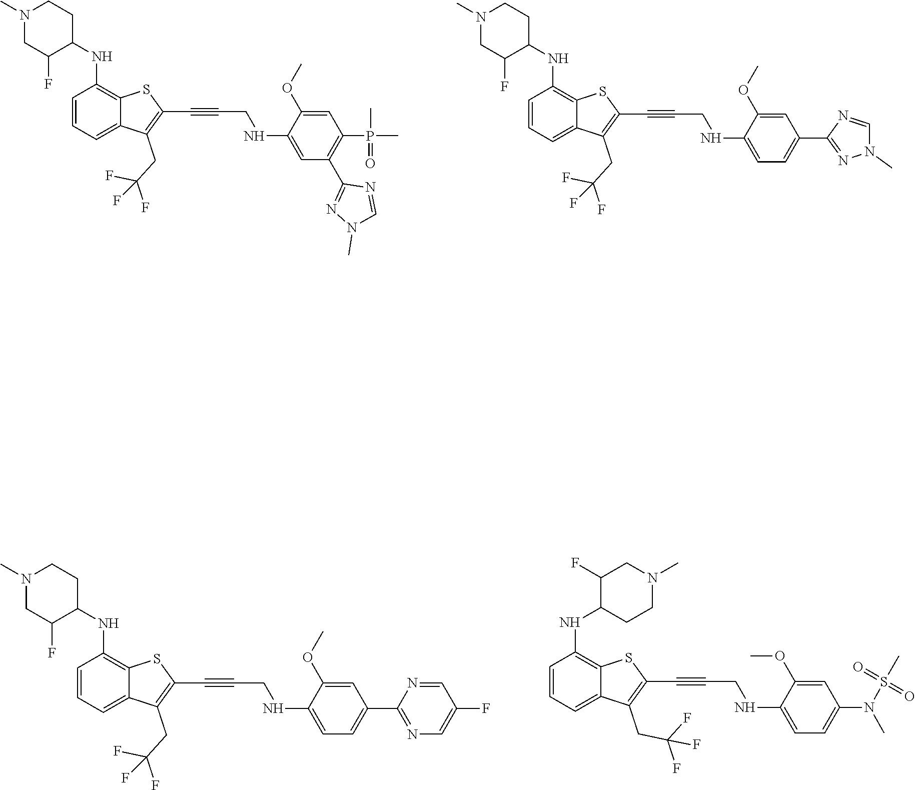

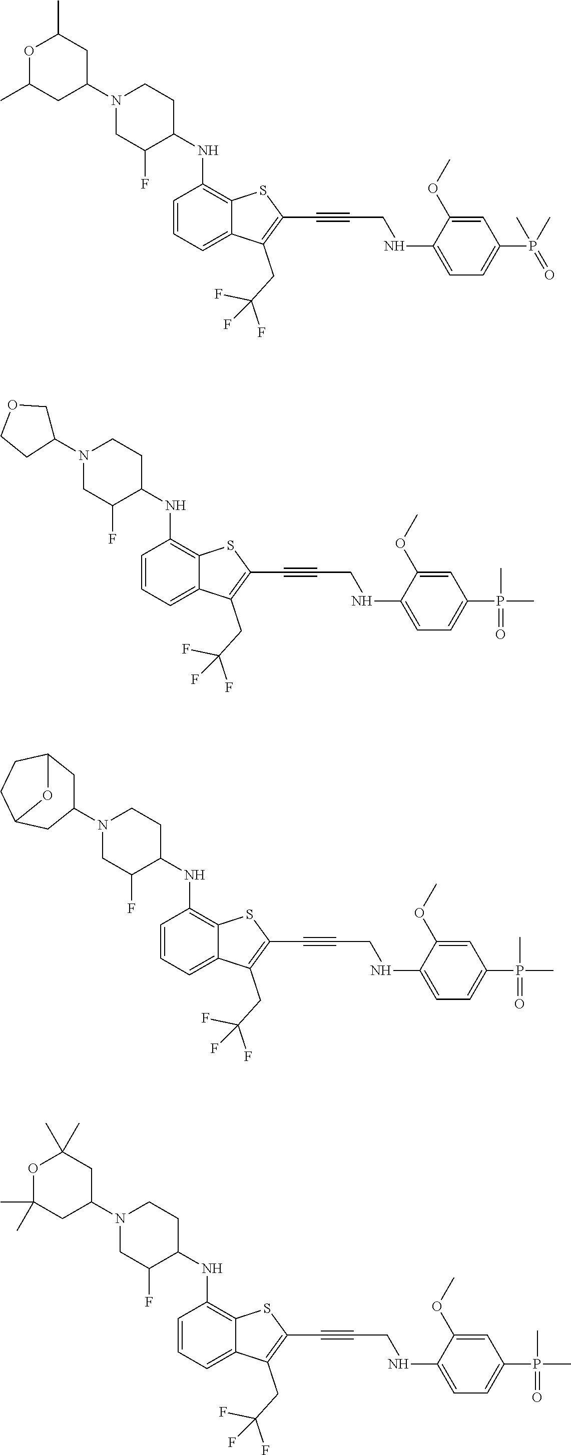

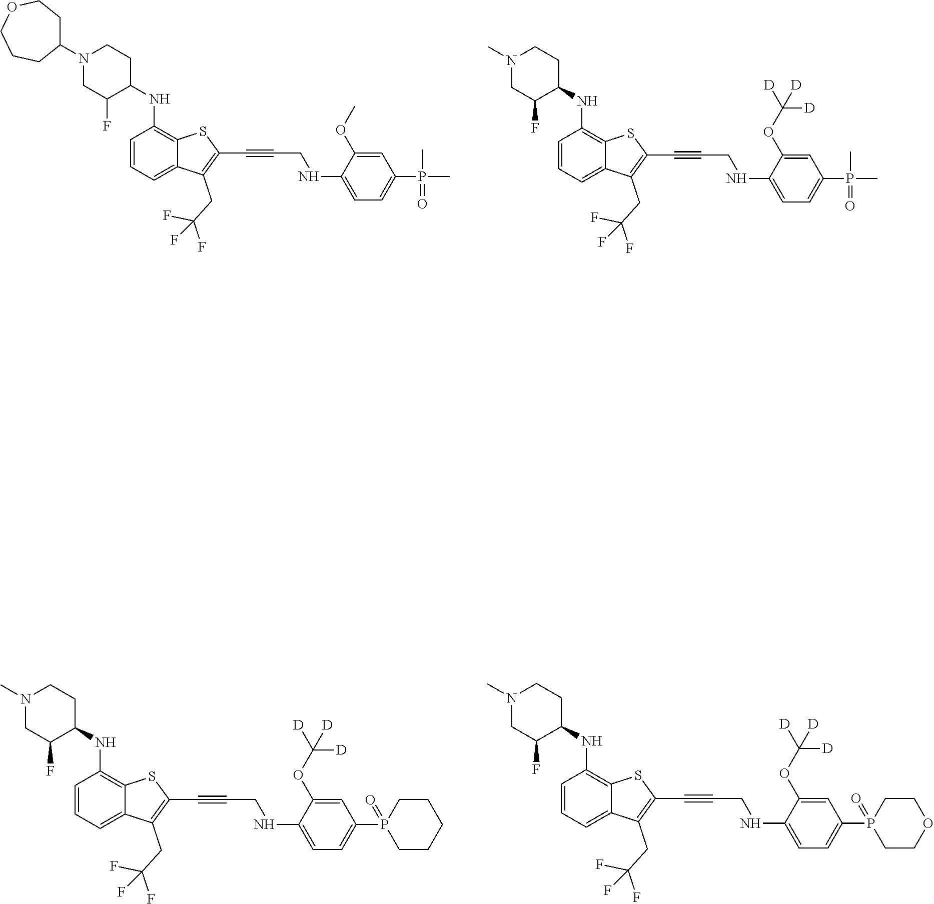

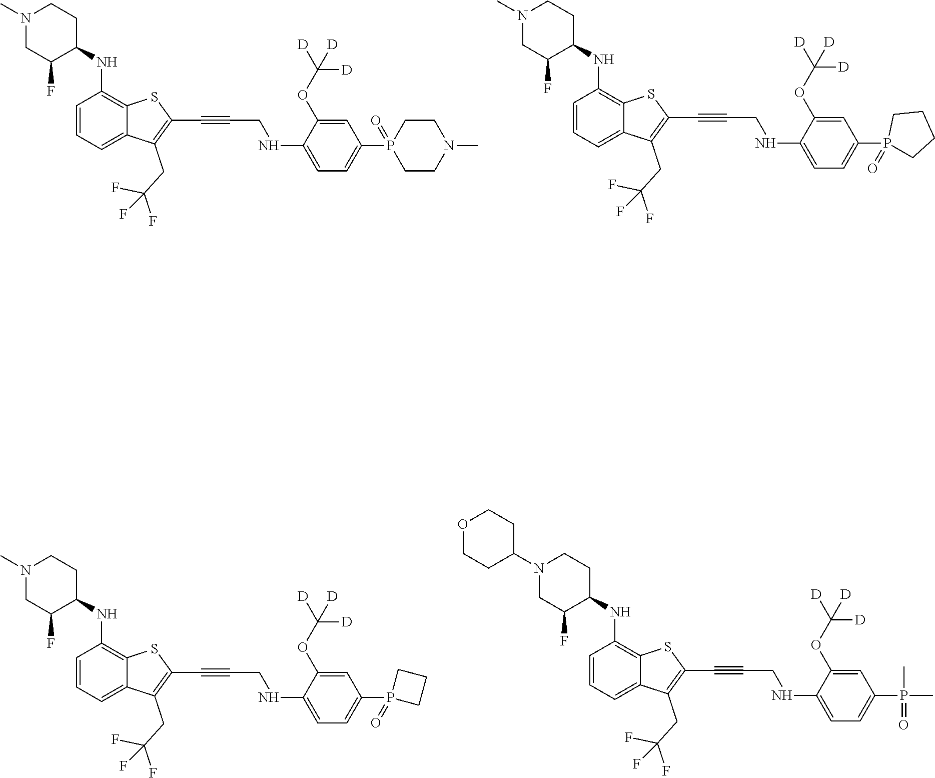

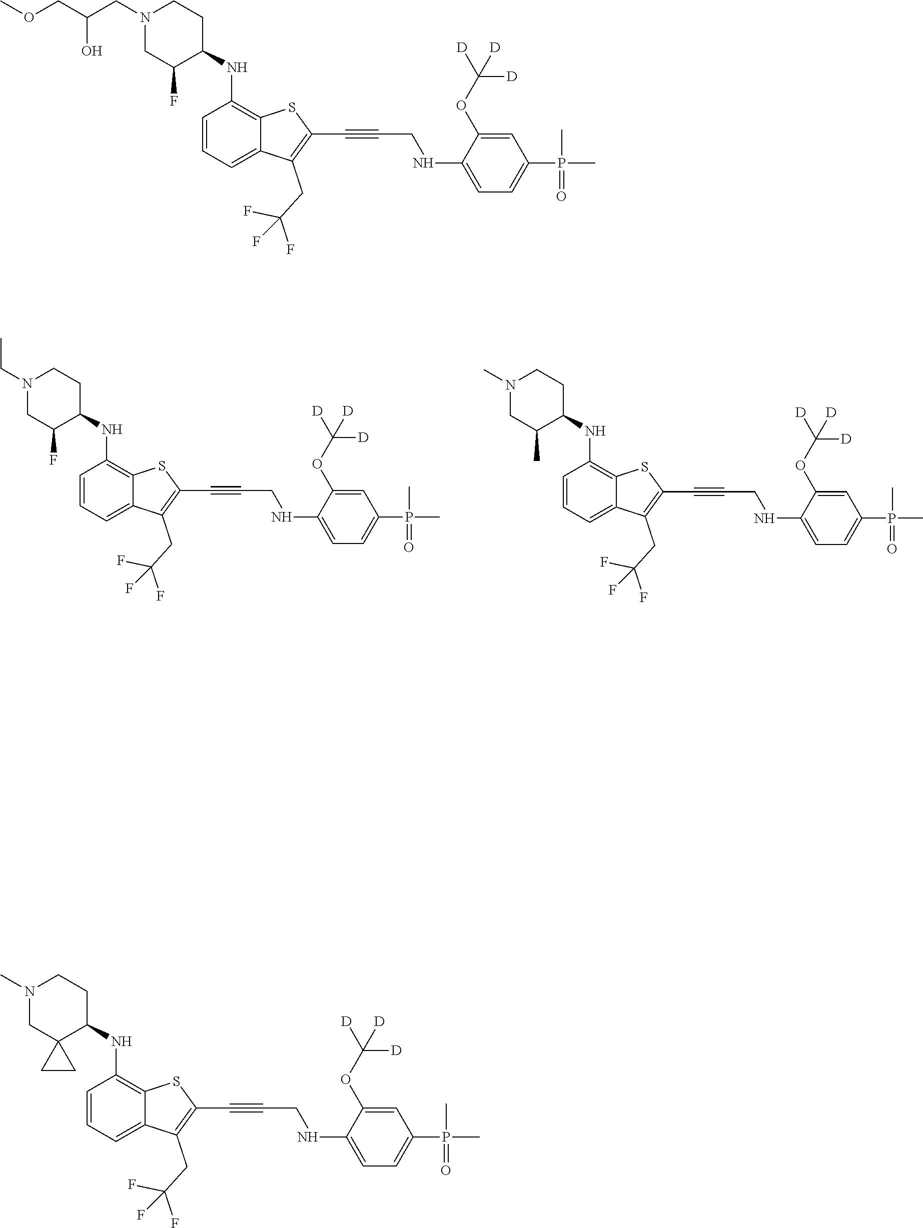

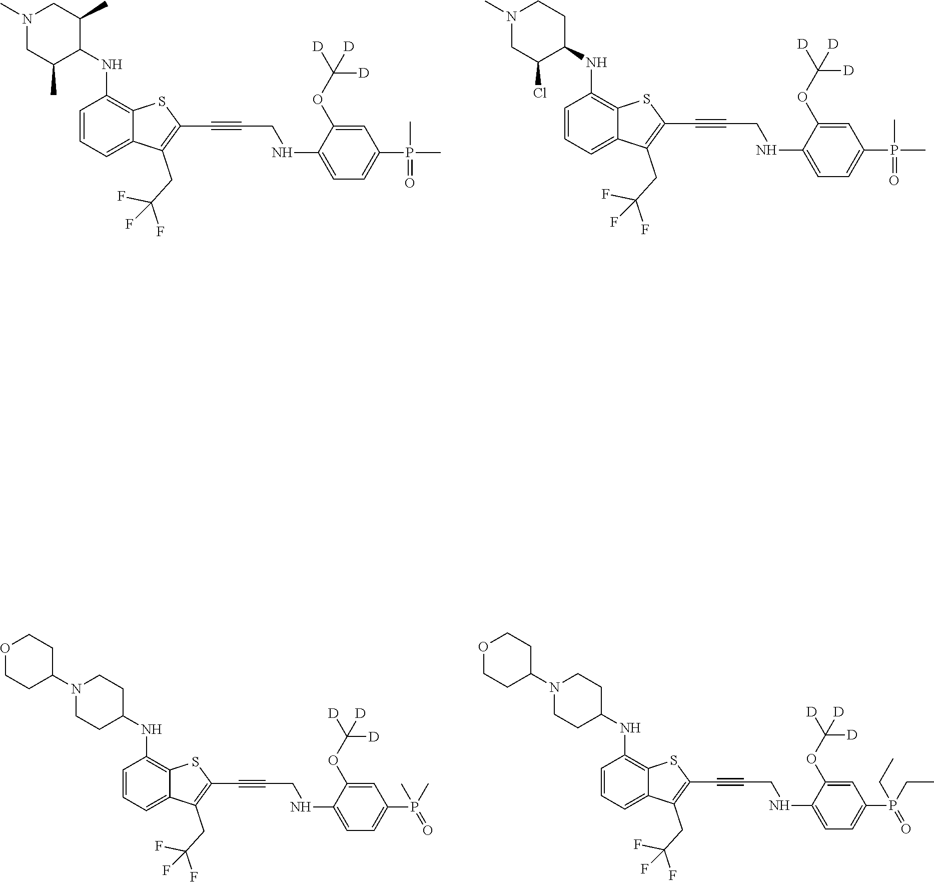

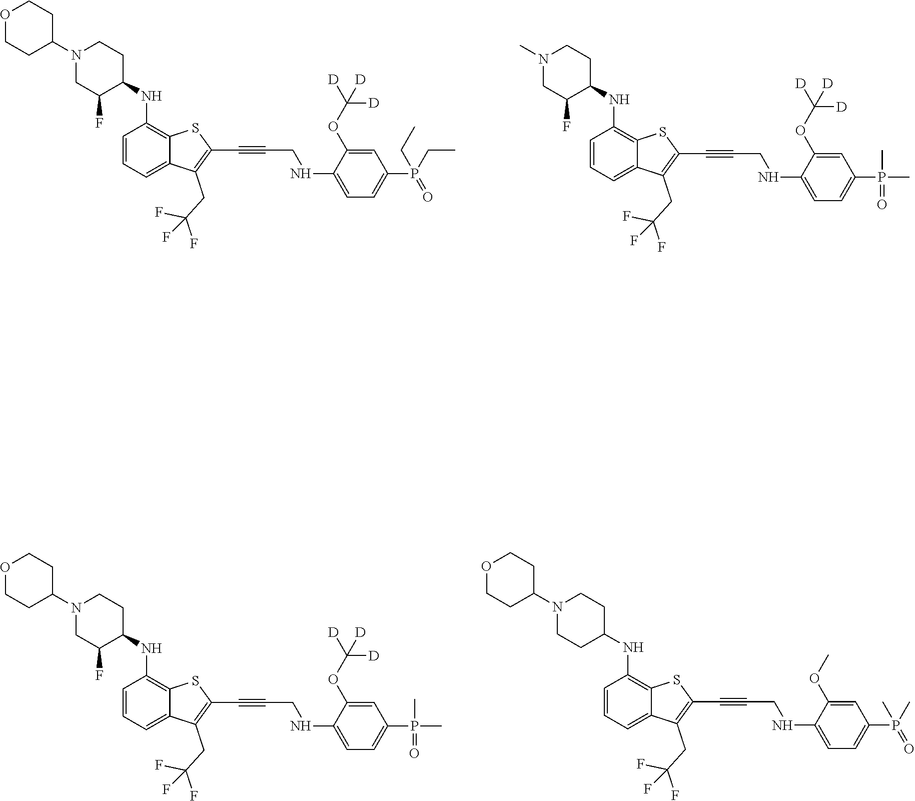

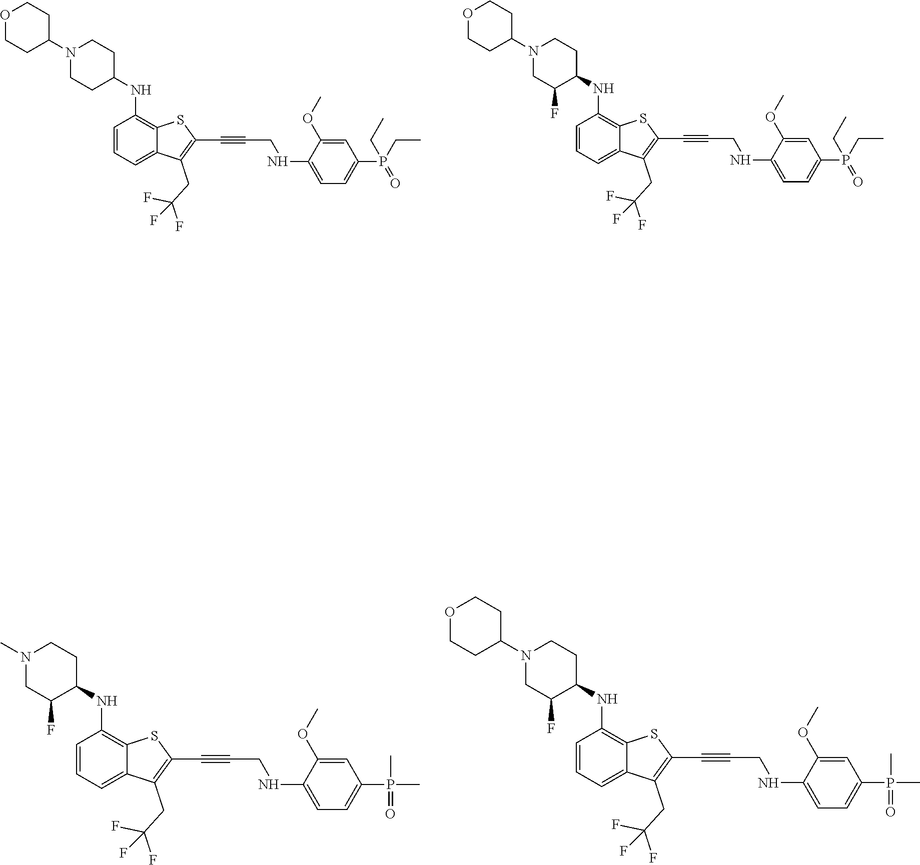

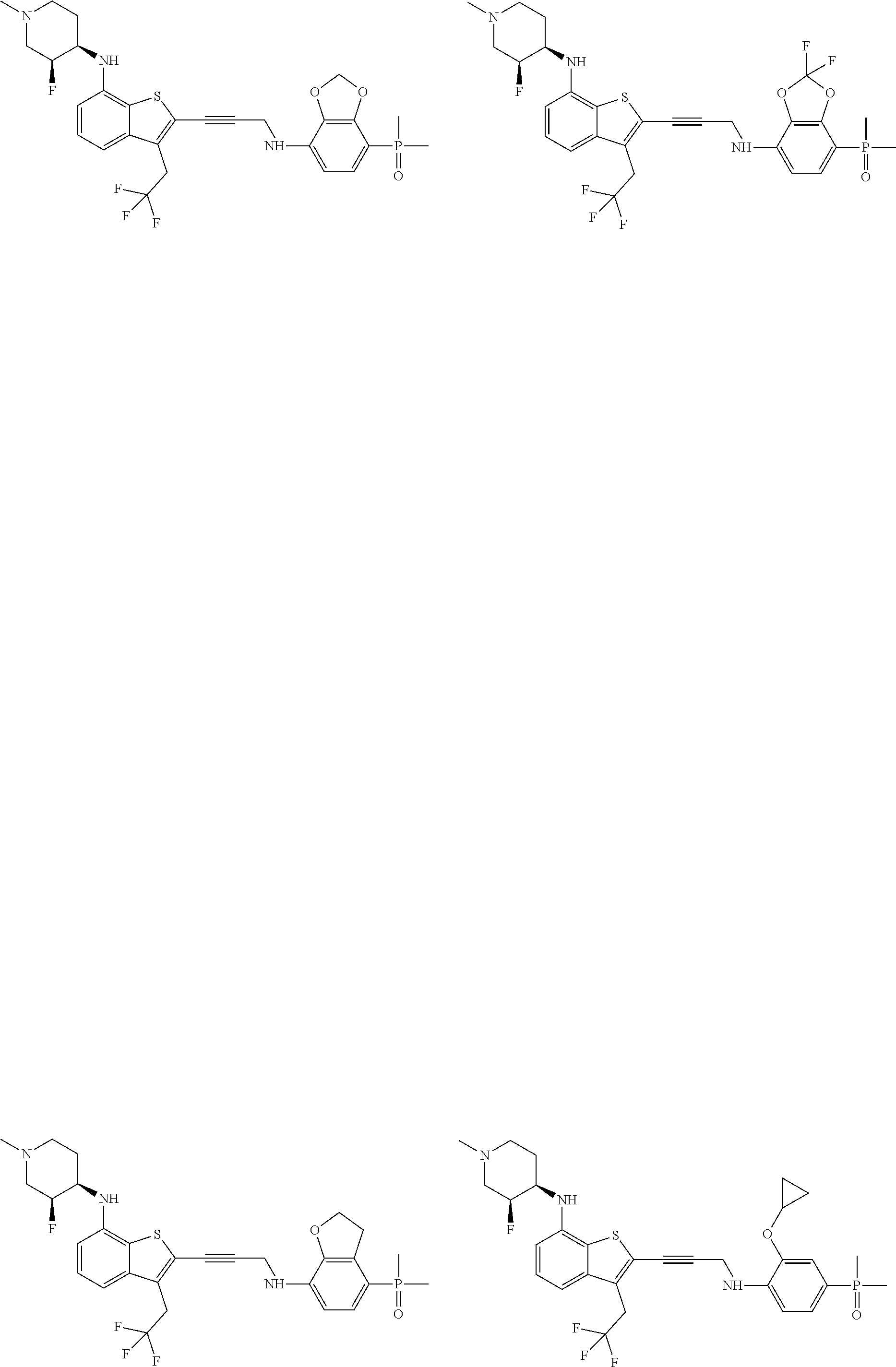

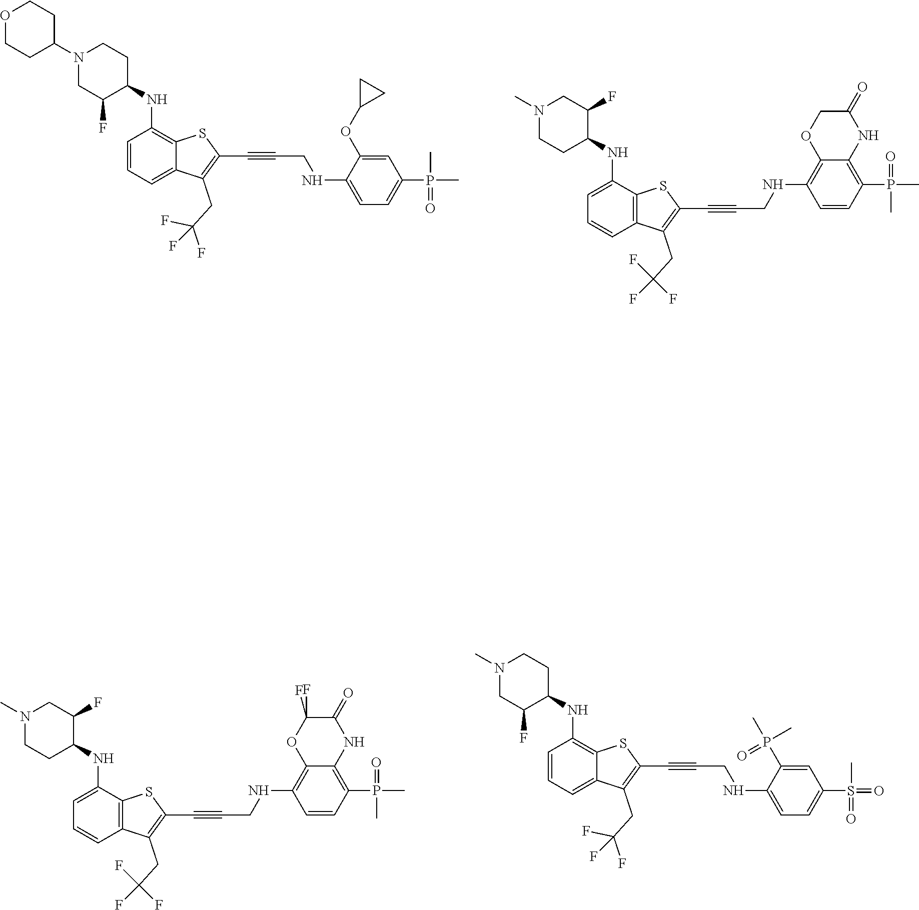

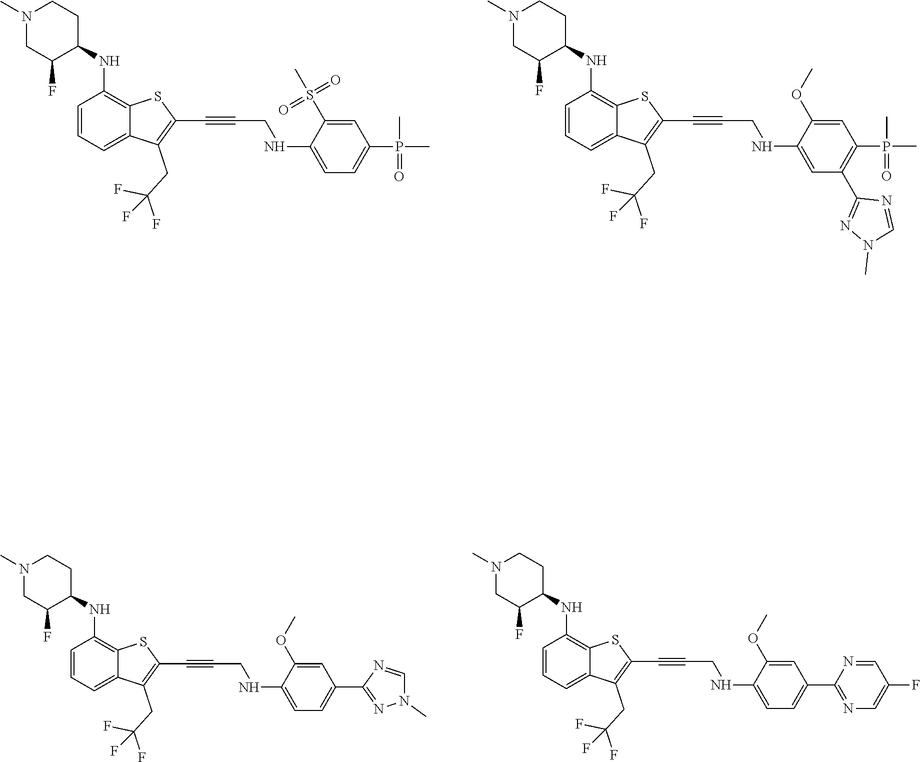

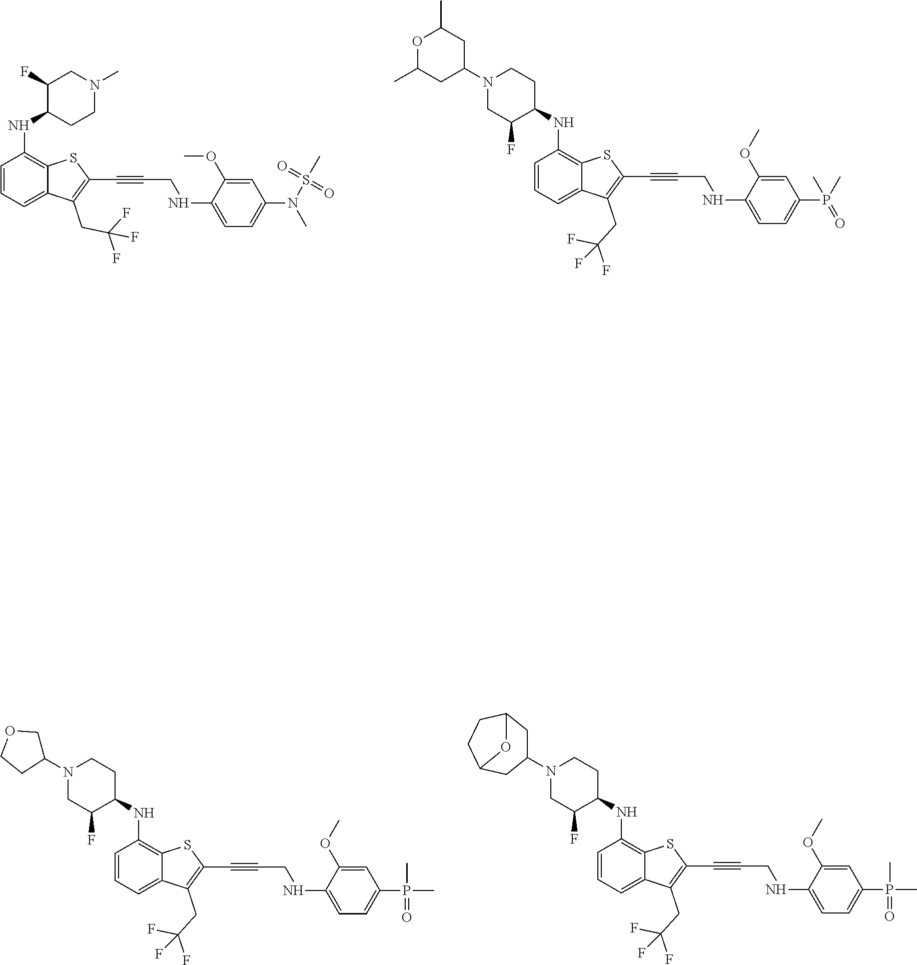

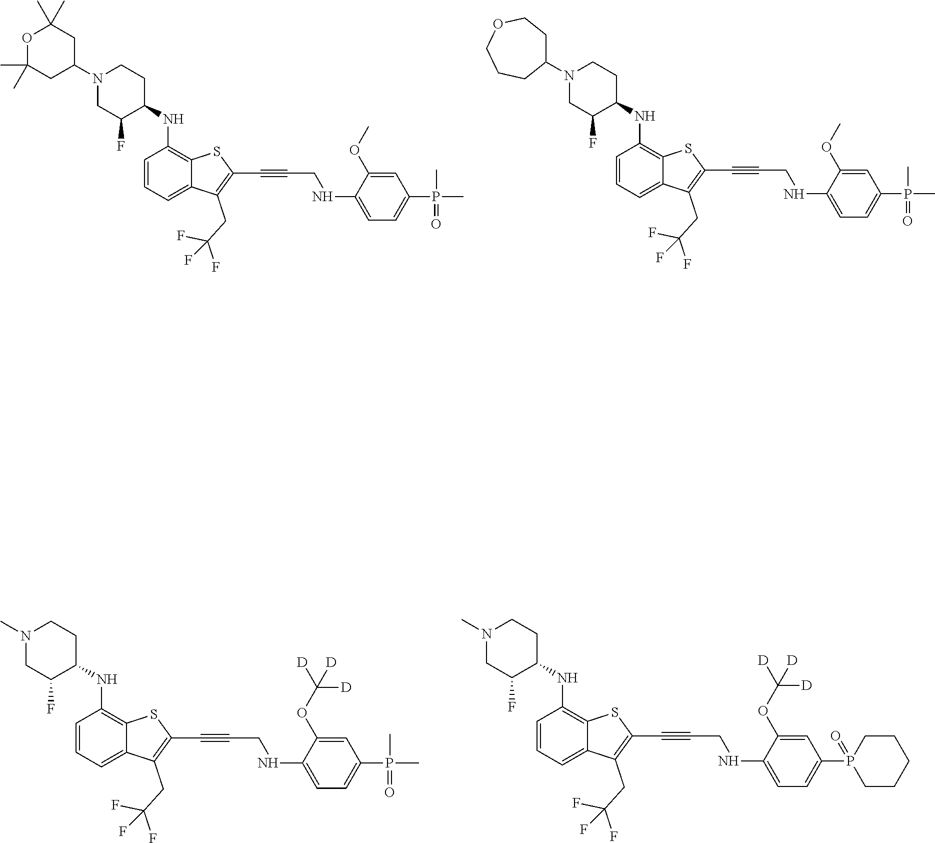

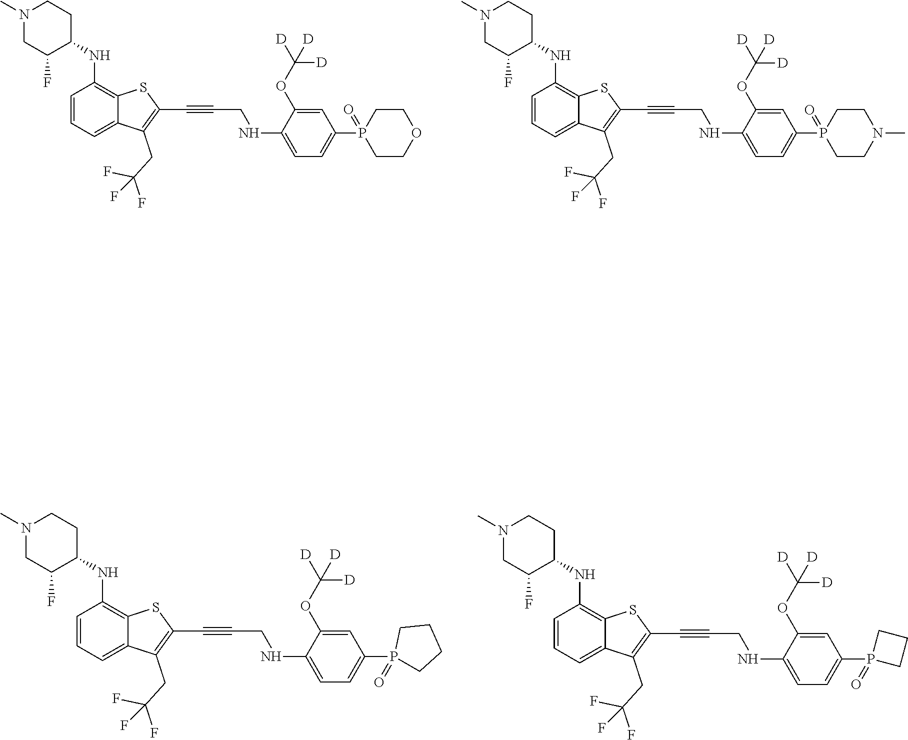

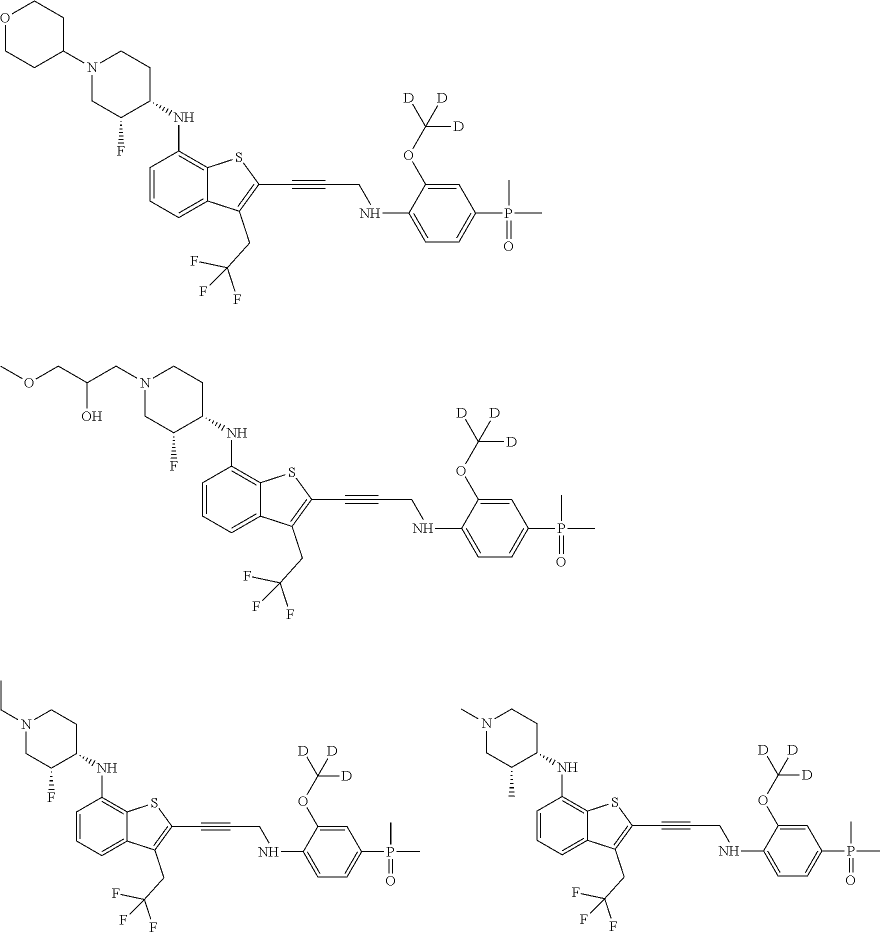

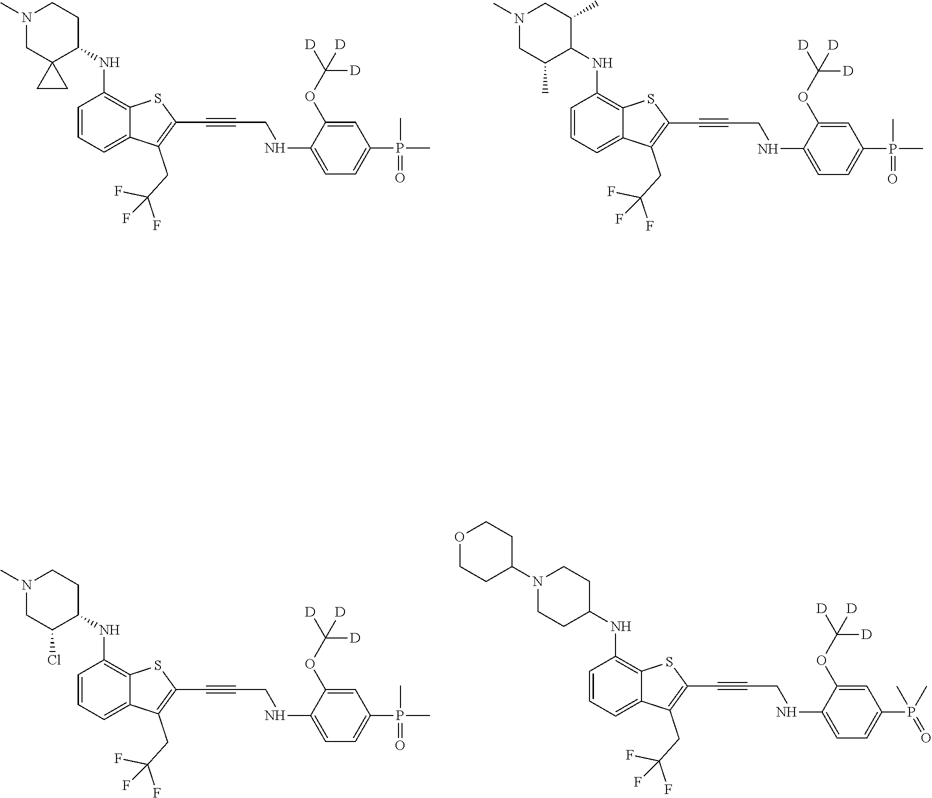

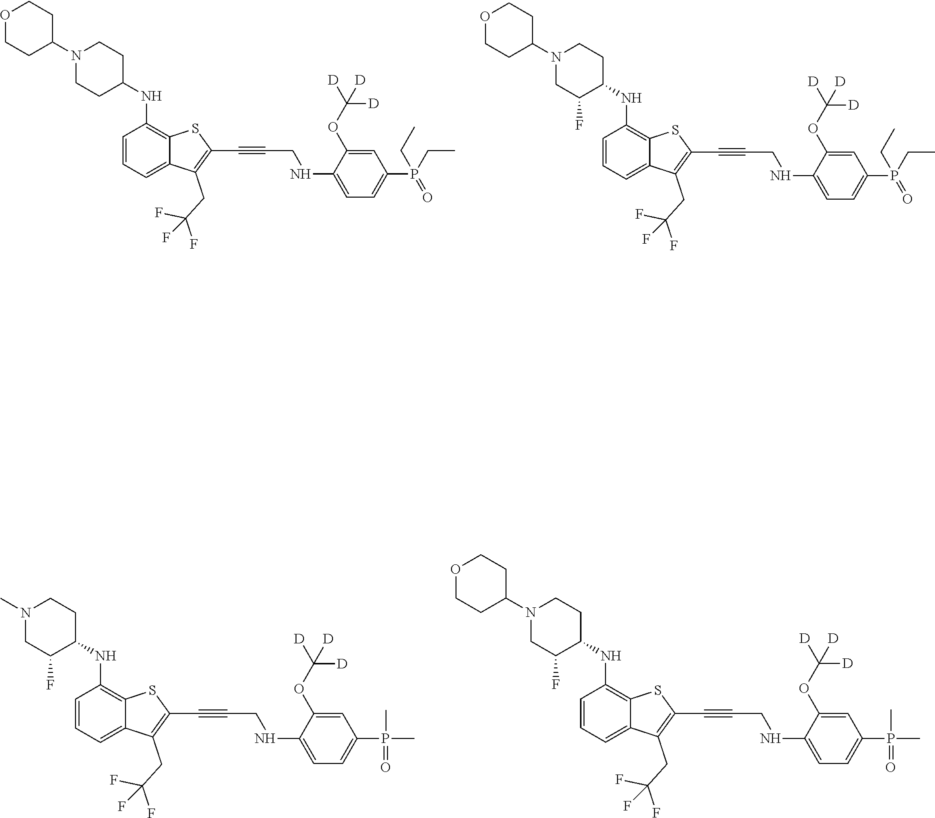

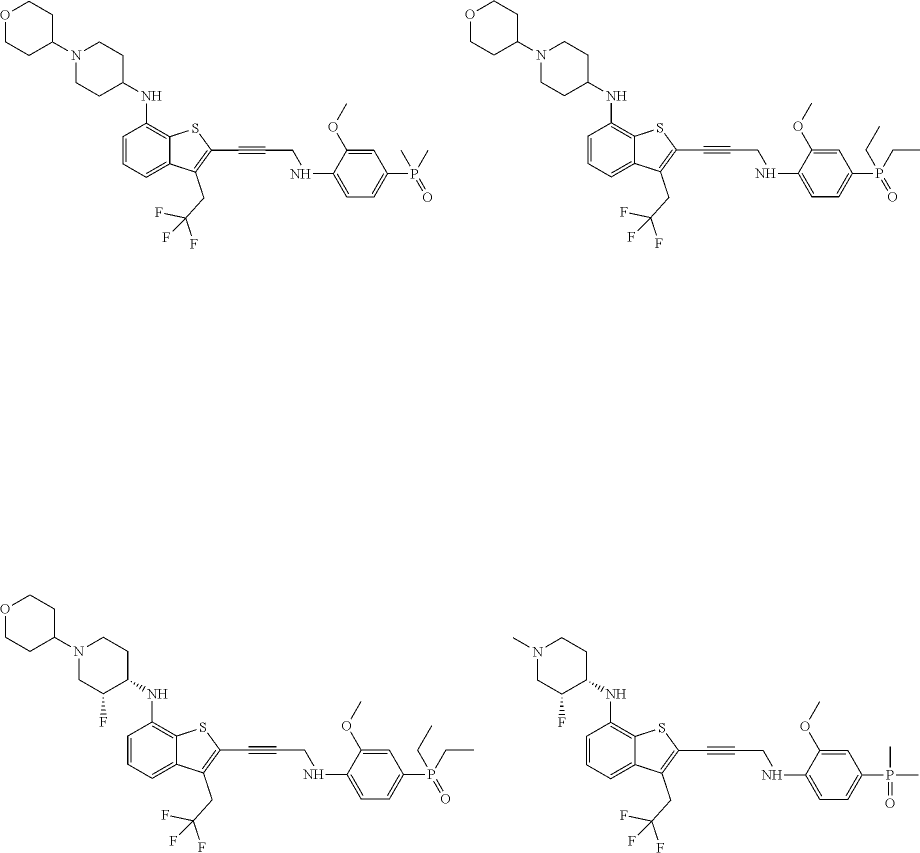

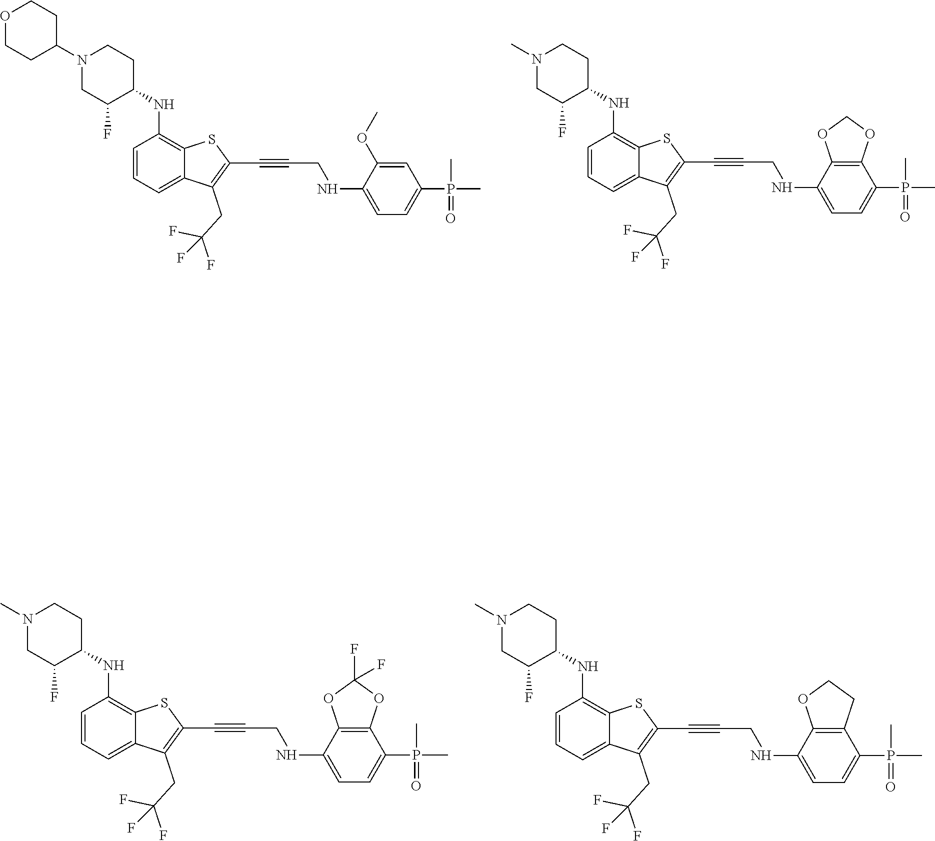

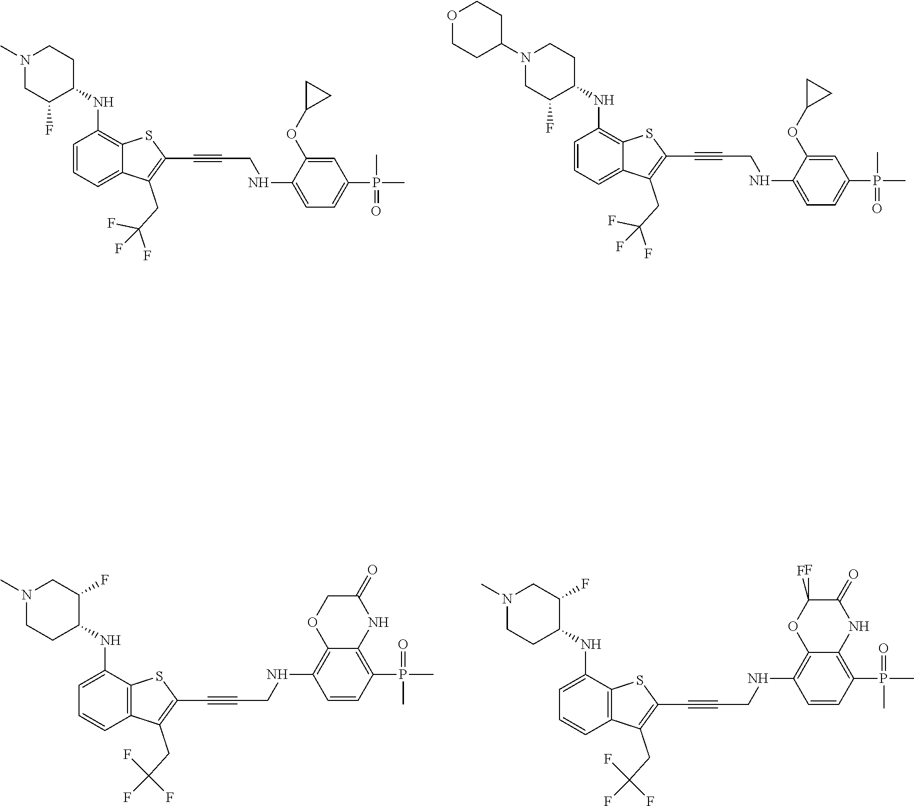

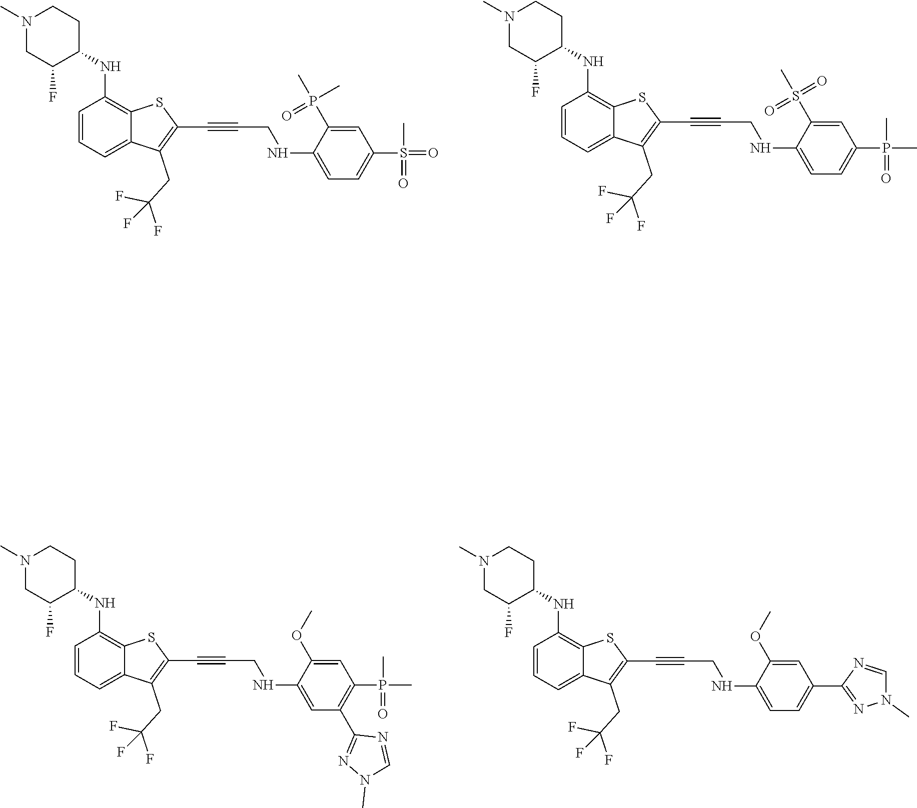

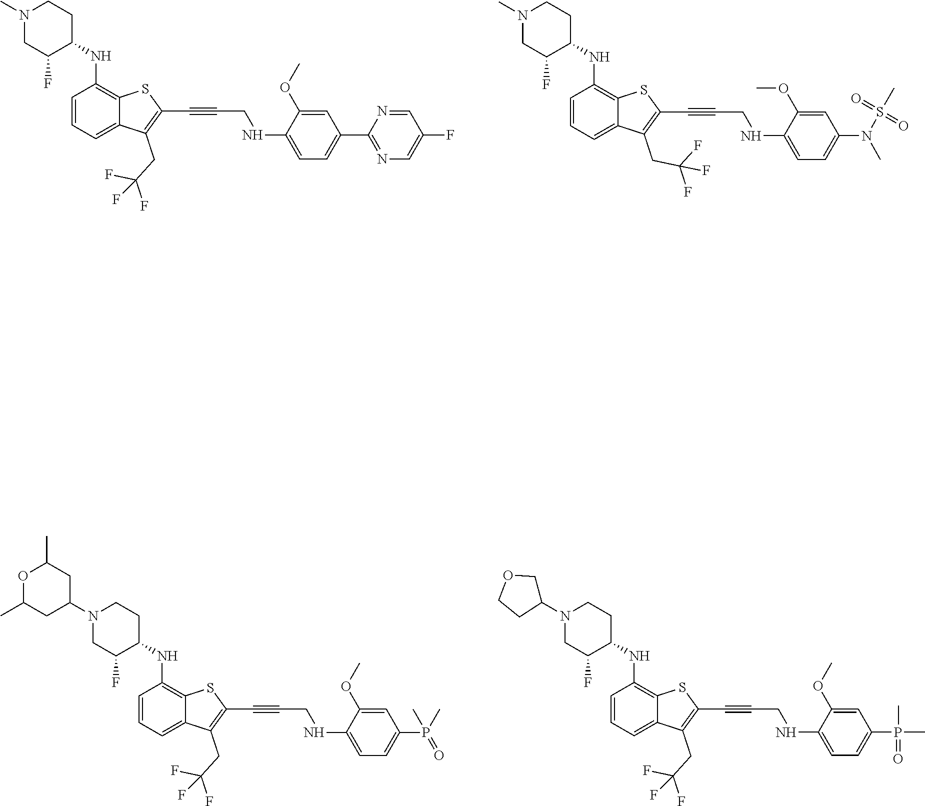

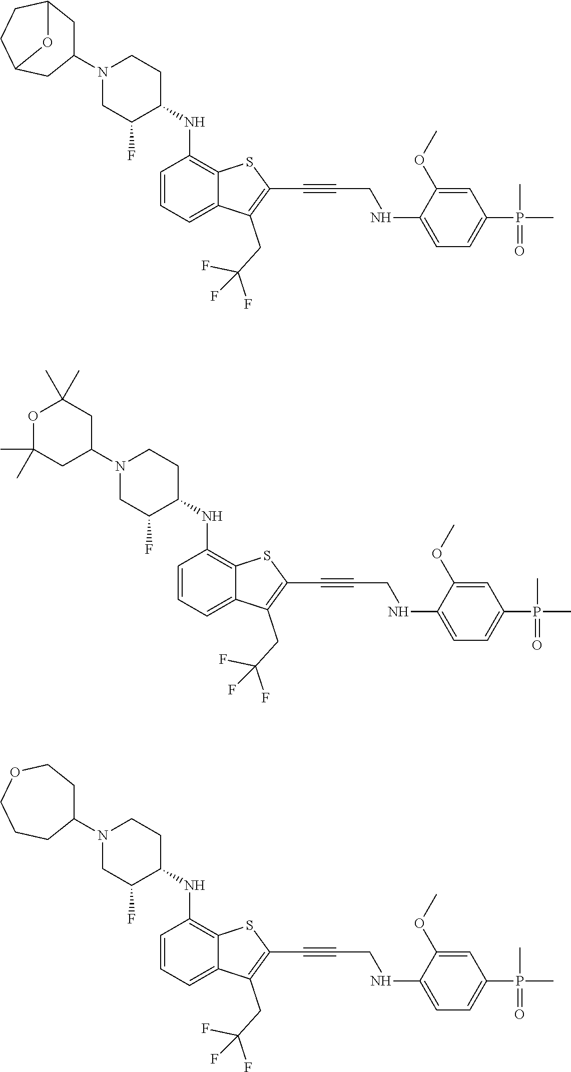

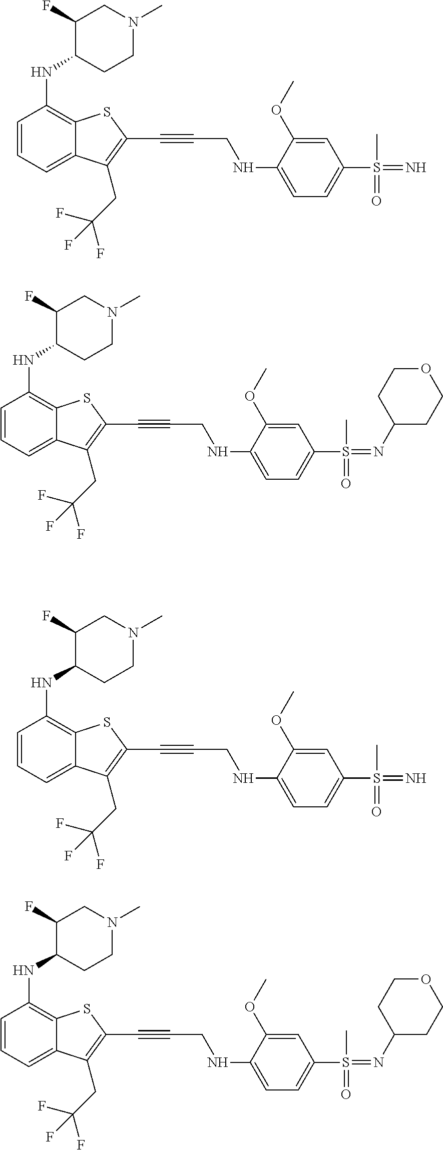 [SMILES: COc1cc(S(C)(=N)=O)ccc1NCC#Cc1sc2c(N[C@H]3CCN(C)C[C@@H]3F)cccc2c1CC(F)(F)F ; COc1cc(S(C)(=O)=NC2CCOCC2)ccc1NCC#Cc1sc2c(N[C@H]3CCN(C)C[C@@H]3F)cccc2c1CC(F)(F)F ; COc1cc(S(C)(=N)=O)ccc1NCC#Cc1sc2c(N[C@@H]3CCN(C)C[C@@H]3F)cccc2c1CC(F)(F)F ; COc1cc(S(C)(=O)=NC2CCOCC2)ccc1NCC#Cc1sc2c(N[C@@H]3CCN(C)C[C@@H]3F)cccc2c1CC(F)(F)F]

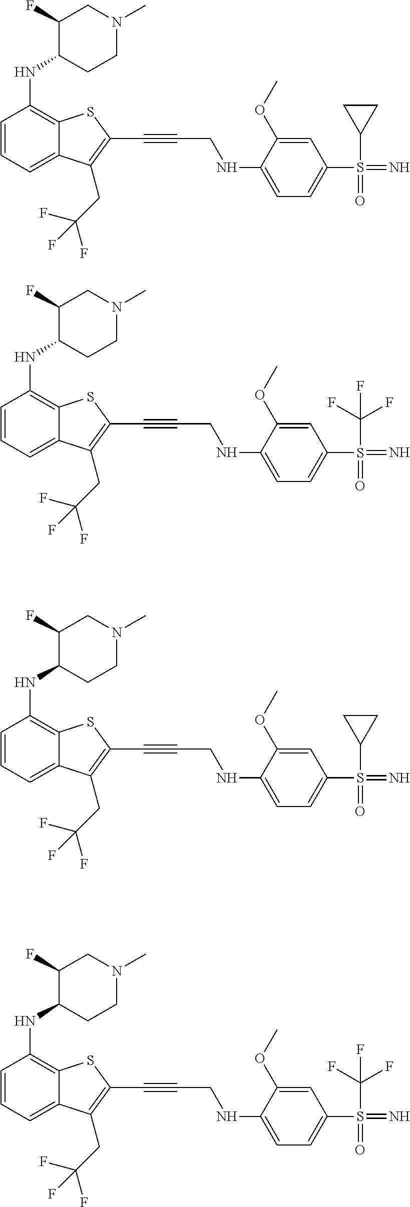

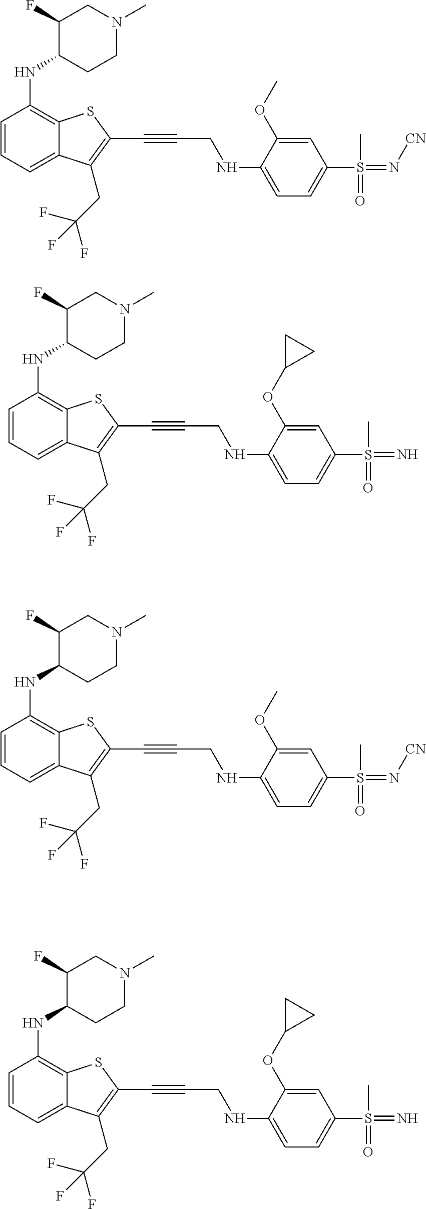

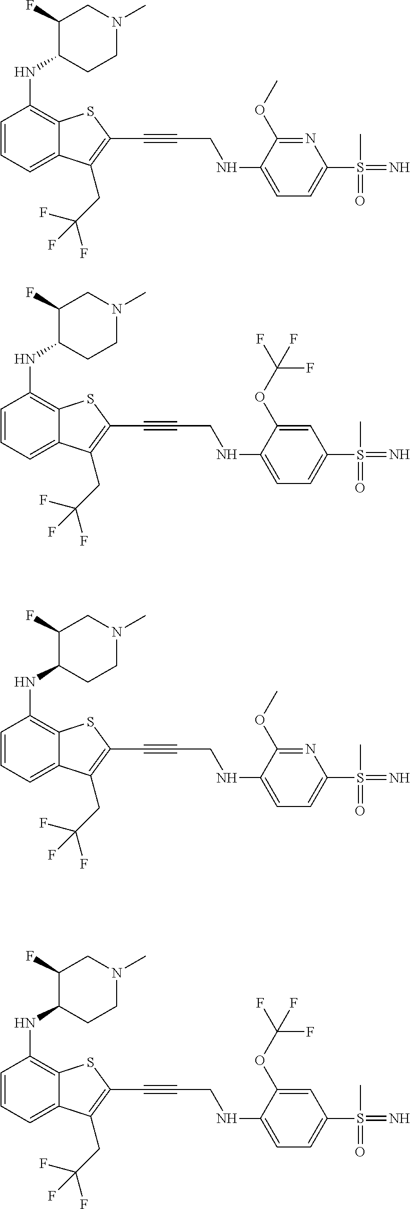

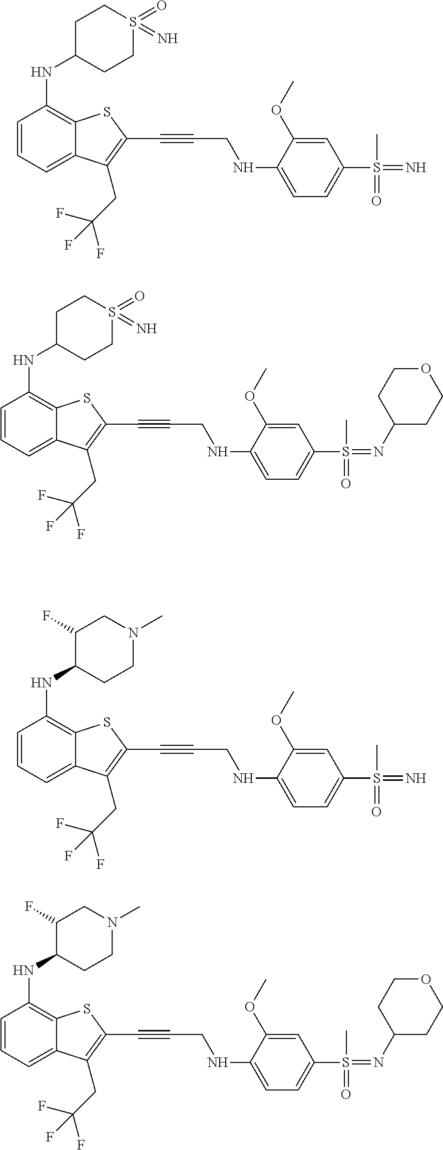

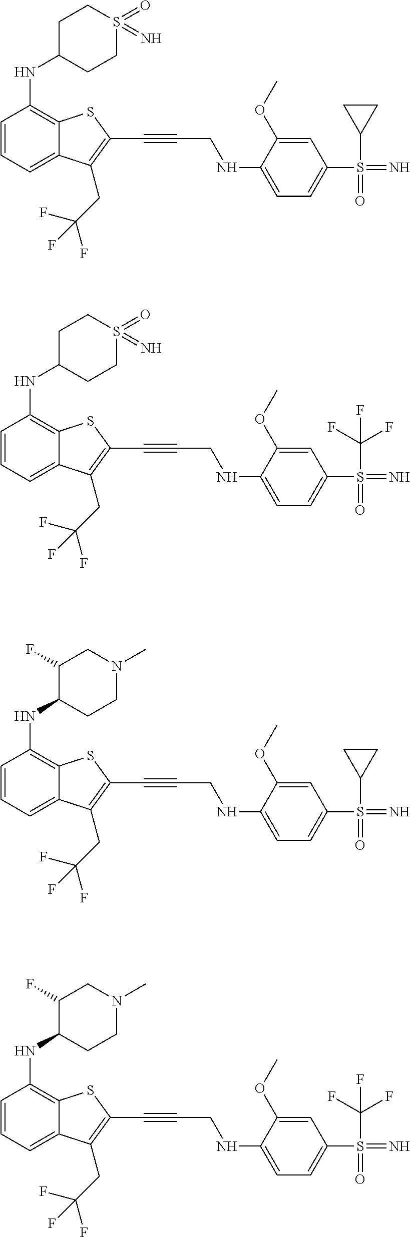

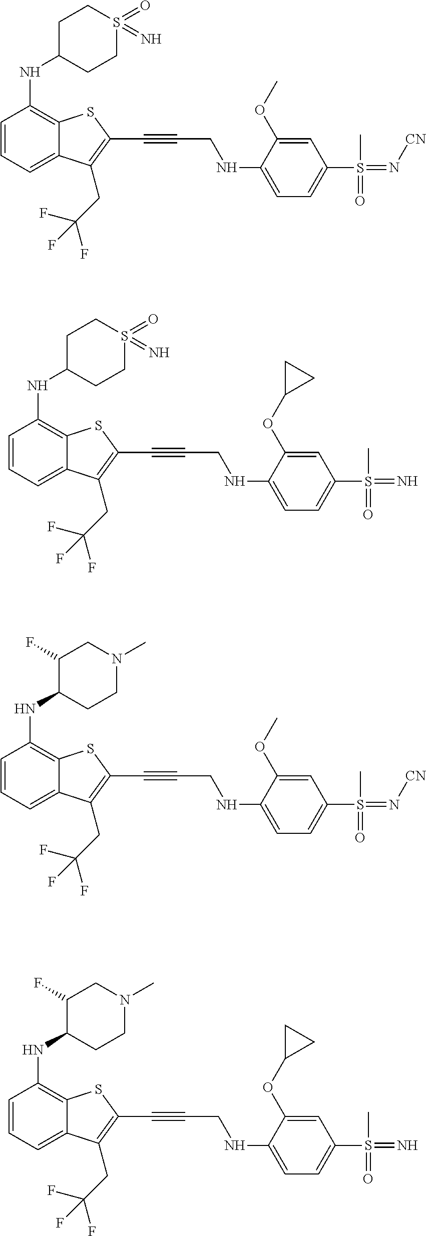

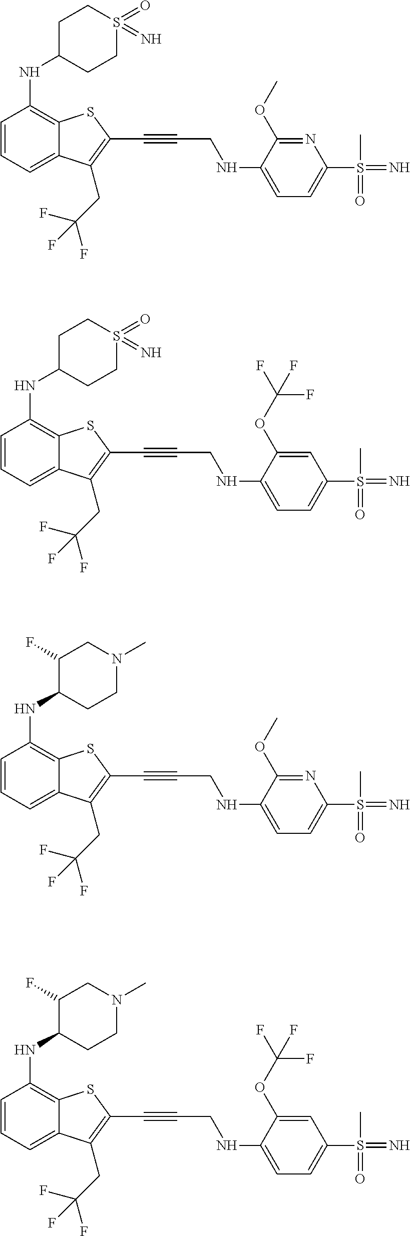

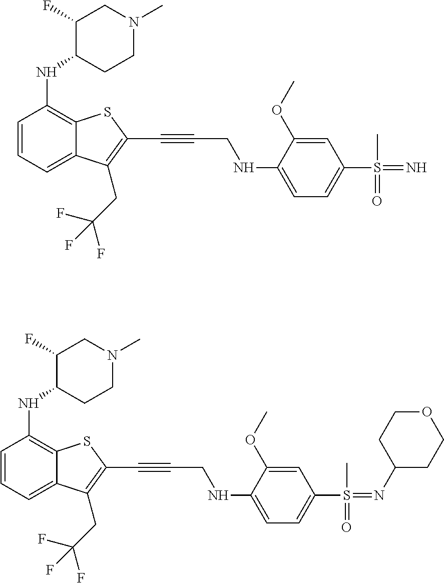 [SMILES: COc1cc(S(C)(=N)=O)ccc1NCC#Cc1sc2c(N[C@H]3CCN(C)C[C@H]3F)cccc2c1CC(F)(F)F ; COc1cc(S(C)(=O)=NC2CCOCC2)ccc1NCC#Cc1sc2c(N[C@H]3CCN(C)C[C@H]3F)cccc2c1CC(F)(F)F]

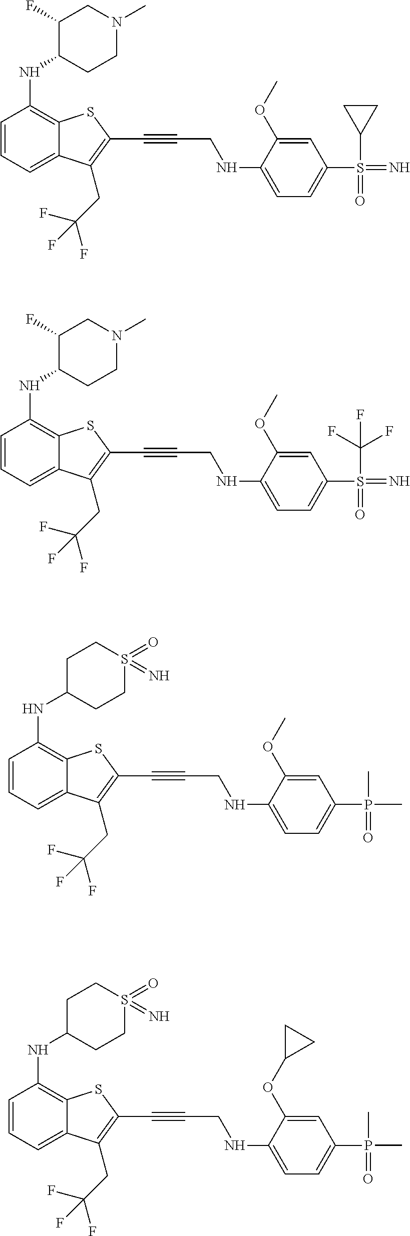 [SMILES: COc1cc(S(=N)(=O)C2CC2)ccc1NCC#Cc1sc2c(N[C@H]3CCN(C)C[C@H]3F)cccc2c1CC(F)(F)F ; COc1cc(S(=N)(=O)C(F)(F)F)ccc1NCC#Cc1sc2c(N[C@H]3CCN(C)C[C@H]3F)cccc2c1CC(F)(F)F ; COc1cc(P(C)(C)=O)ccc1NCC#Cc1sc2c(NC3CCS(=N)(=O)CC3)cccc2c1CC(F)(F)F ; CP(C)(=O)c1ccc(NCC#Cc2sc3c(NC4CCS(=N)(=O)CC4)cccc3c2CC(F)(F)F)c(OC2CC2)c1]

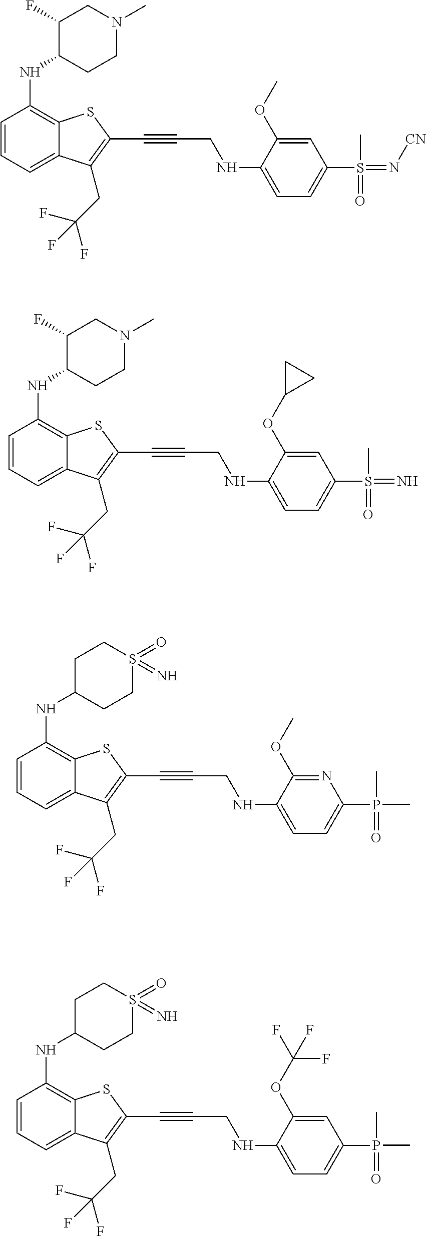 [SMILES: COc1cc(S(C)(=O)=NC#N)ccc1NCC#Cc1sc2c(N[C@H]3CCN(C)C[C@H]3F)cccc2c1CC(F)(F)F ; CN1CC[C@H](Nc2cccc3c(CC(F)(F)F)c(C#CCNc4ccc(S(C)(=N)=O)cc4OC4CC4)sc23)[C@H](F)C1 ; COc1nc(P(C)(C)=O)ccc1NCC#Cc1sc2c(NC3CCS(=N)(=O)CC3)cccc2c1CC(F)(F)F ; CP(C)(=O)c1ccc(NCC#Cc2sc3c(NC4CCS(=N)(=O)CC4)cccc3c2CC(F)(F)F)c(OC(F)(F)F)c1]

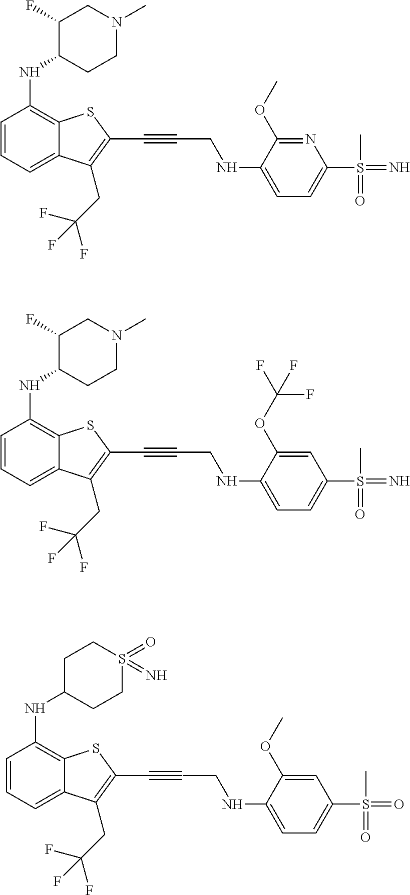 [SMILES: COc1nc(S(C)(=N)=O)ccc1NCC#Cc1sc2c(N[C@H]3CCN(C)C[C@H]3F)cccc2c1CC(F)(F)F ; CN1CC[C@H](Nc2cccc3c(CC(F)(F)F)c(C#CCNc4ccc(S(C)(=N)=O)cc4OC(F)(F)F)sc23)[C@H](F)C1 ; COc1cc(S(C)(=O)=O)ccc1NCC#Cc1sc2c(NC3CCS(=N)(=O)CC3)cccc2c1CC(F)(F)F]

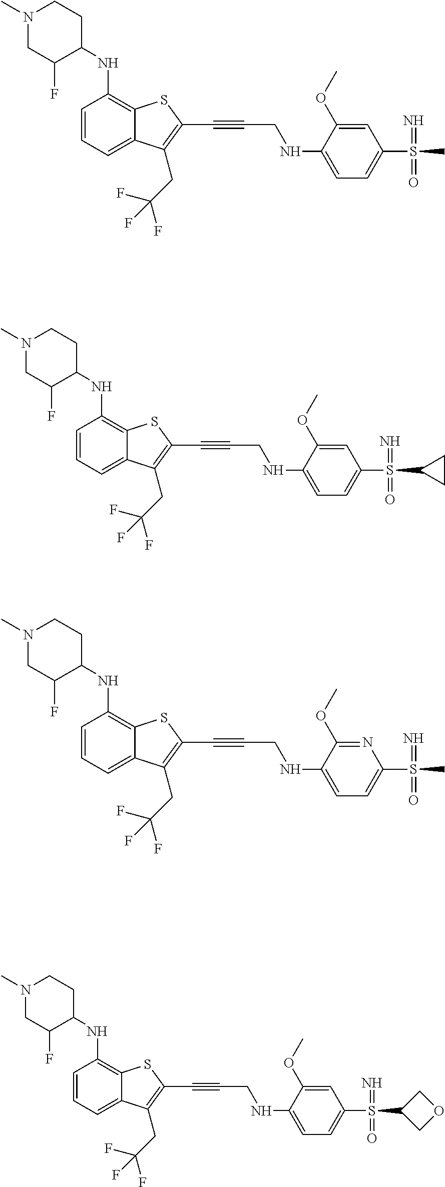

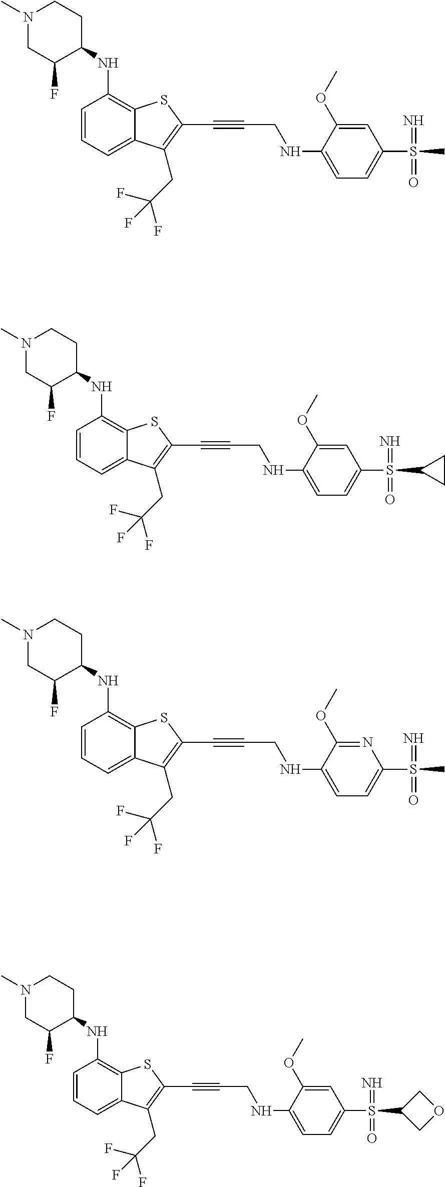

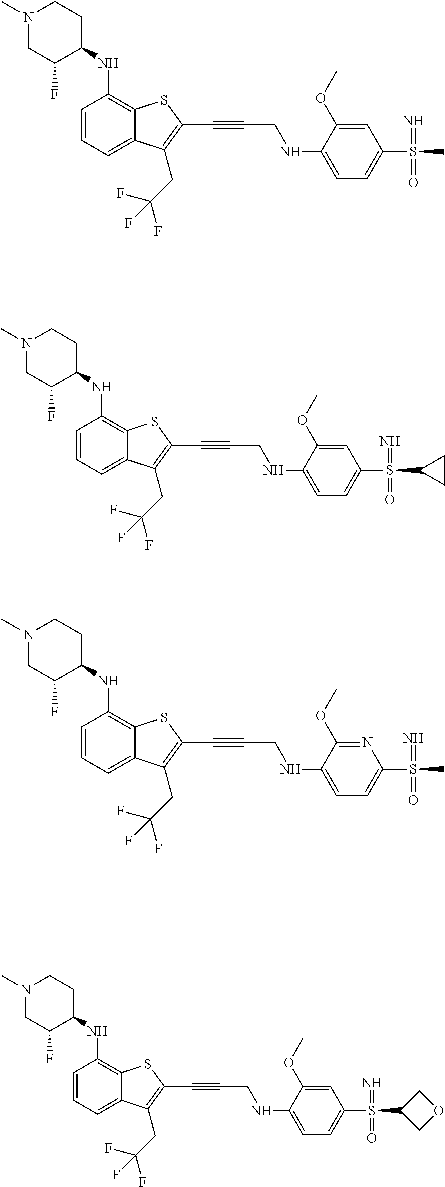

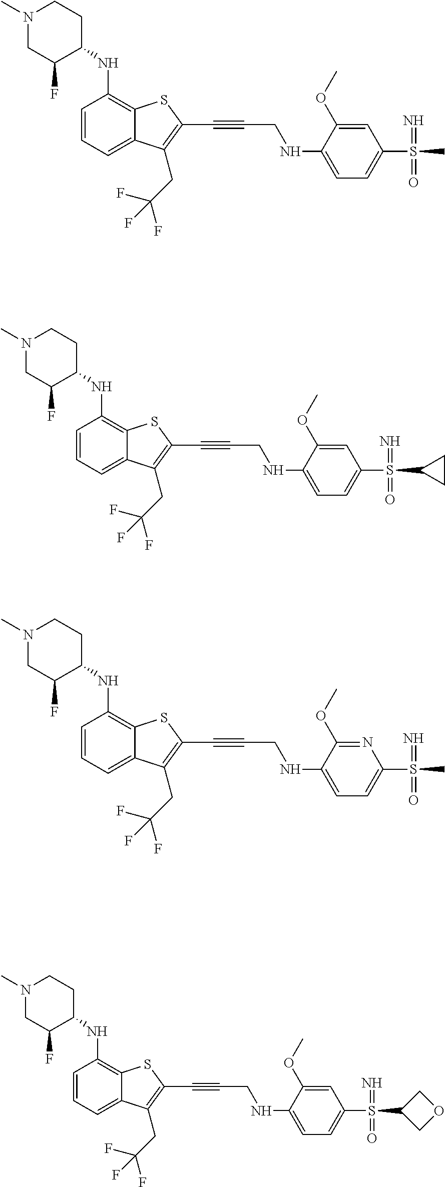

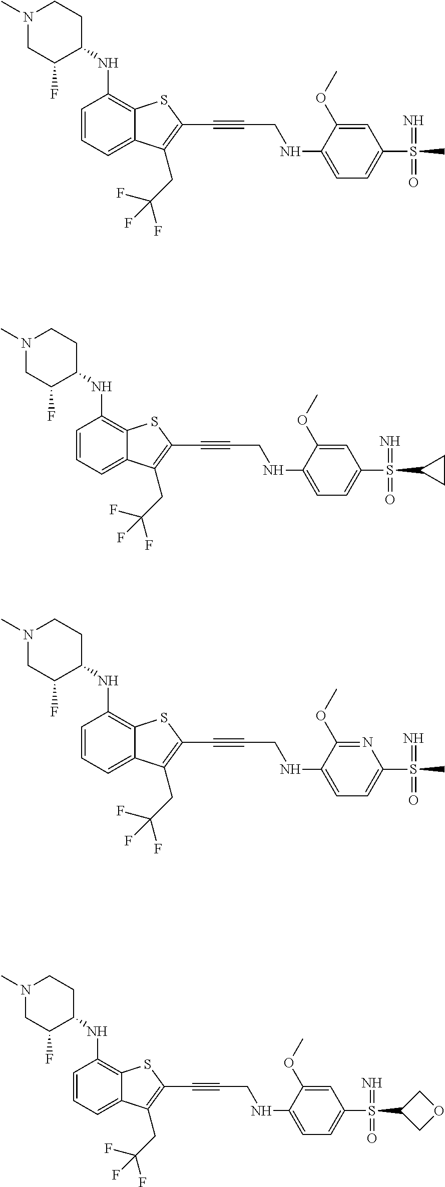

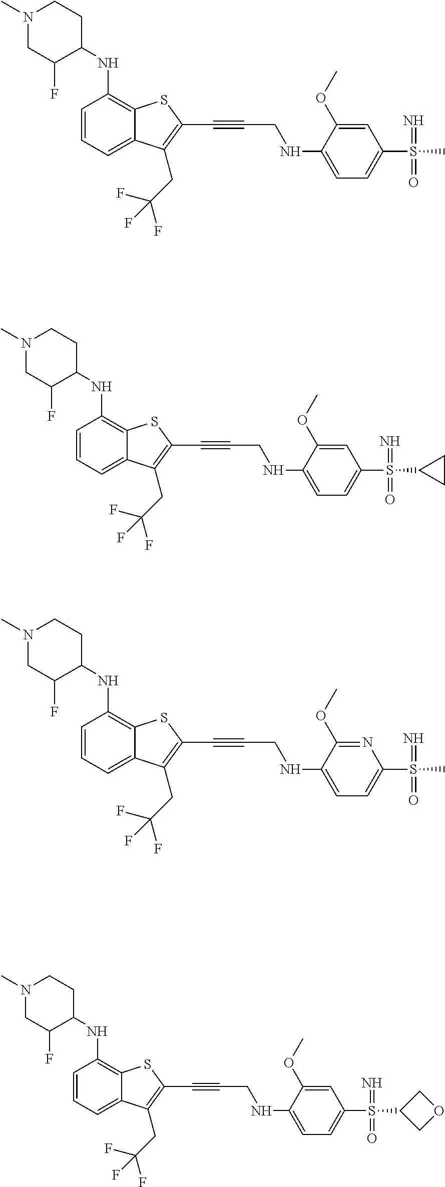

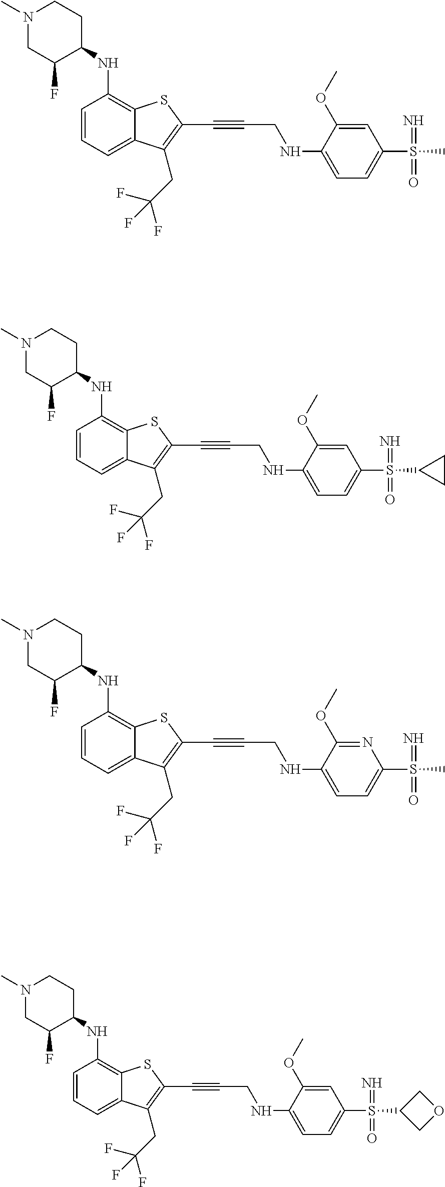

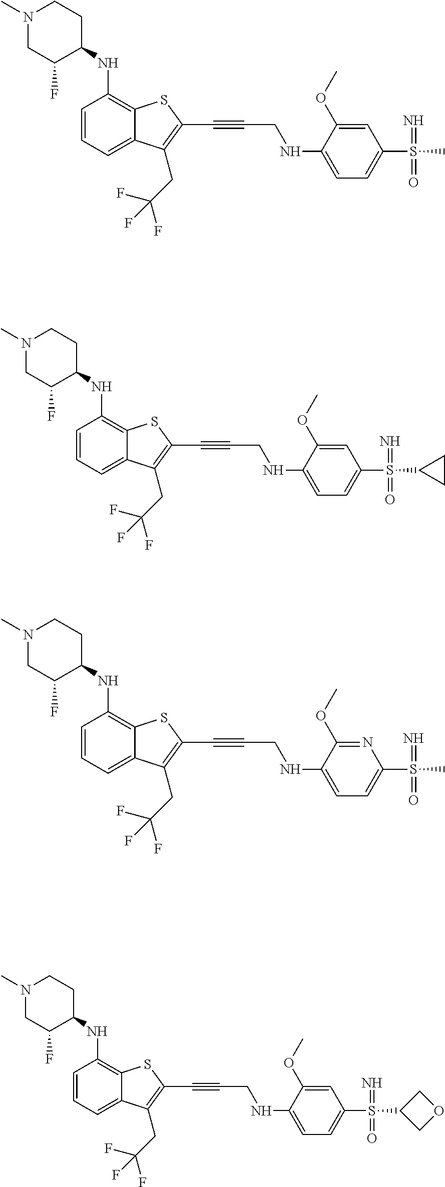

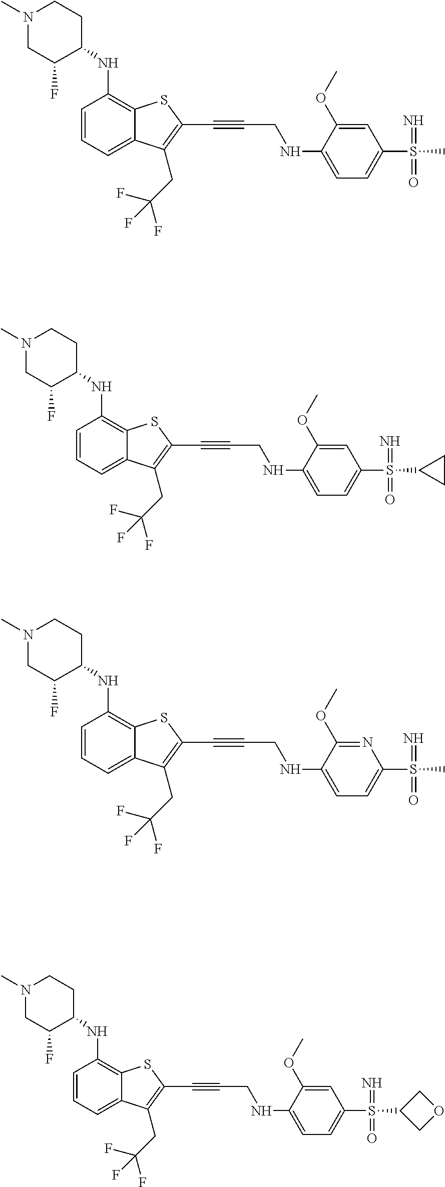

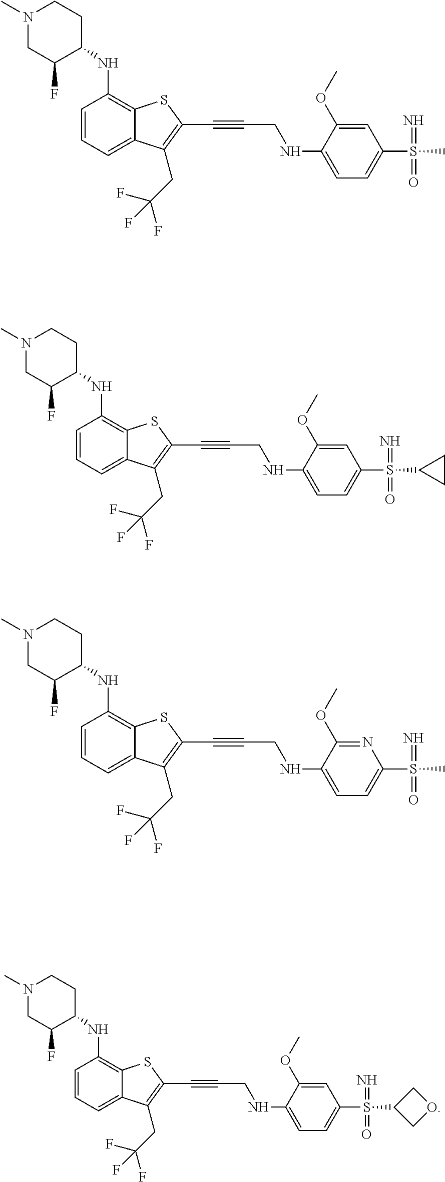

103. A compound of formula (IN—I): 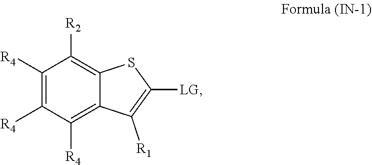
wherein R₁, R₂ or R₄ is defined in the same manner as claim 83, and LG is selected from a leaving group, such as halogen, preferably, bromine or iodine.

104. A method for preparation of the compound of formula (I-1) according to claim 83, comprising steps of: 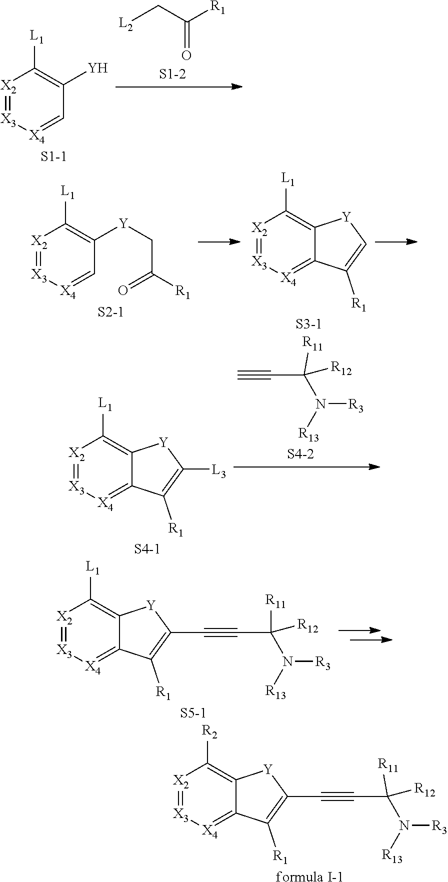
(1) reacting a compound S1-1 as starting material with a compound S1-2 in the presence of an alkaline agent to form a compound of S2-1;
(2) converting the compound of S2-1 into a compound S3-1 under acidic condition;
(3) converting the compound S3-1 in the presence of halogenating reagent into a compound S4-1;
(4) reacting the compound S4-1 with reagent S4-2 in the presence of a coupling catalyst to form a compound S5-1;
(5) converting the compound S5-1 into the compound of formula (I-1) through one or more steps of reaction such as a reductive amination reaction, a deprotecting reaction or a combination thereof;
wherein L₁, L₂ and L₃ independently represent a leaving group;
Y is selected from O or S;
X₂, X₃, X₄, R₁, R₂, R₁₁, R₁₂, R₁₃, R₃, R₅₁, R₅₂ and m are each defined as claim 1.

105. The method according to claim 104, wherein the alkaline agent is K₂CO₃;
the acidic condition is polyphosphoric acid;
the halogenating reagent is NIS;
group L₁ of the compound S5-1 is substituted with —NH₂, and then further converted to the compound of formula (I-1) through a reductive amination reaction, a deprotecting reaction or a combination thereof, when R₂ is —NR₅₁R₅₂;
the coupling catalyst comprises Pd-containing coupling catalyst, such as Pd(PPh₃)₂Cl₂ or Pd(dppf)Cl₂; or
the leaving group is halogen, such as —Cl, —Br or —I.

106. The method according to claim 104, wherein L₁ is selected from —Br; L₂ is selected from —Br; and L₃ is selected from —I.

107. A pharmaceutical composition, comprising a therapeutically effective amount of a compound of formula (I), or a stereoisomer, tautomer, deuterated derivative, prodrug or pharmaceutically acceptable salt thereof according to claim 83, and a pharmaceutically acceptable carrier, diluent, or excipient.

108. A method for preventing or treating a disease or condition related to p53 mutant protein in a subject, comprising administering to a subject a therapeutically effective amount of a compound of formula (I), or a stereoisomer, tautomer, deuterated derivative, prodrug or pharmaceutically acceptable salt thereof according to claim 1; wherein, the disease or condition is cancer.

109. The method according to claim 108, wherein the cancer cell expresses the p53 mutant.

110. The method according to claim 109, wherein the p53 mutant has a mutation at amino acid Val143, His168, Arg175, Tyr220, Gly245, Arg248, Arg249, Phe270, Arg273, Arg282, and/or a combination thereof: or
the p53 mutant is V157F, R175H, Y220C, G245S, R248Q, R248W, R249S, R273H, R273C, R282W, and/or a combination thereof.

111. The method according to claim 109, wherein the p53 mutant is Y220C.

112. The method according to claim 108, wherein the disease or condition is selected from the group consisting of ovarian cancer, breast cancer, lung cancer and/or a combination thereof.
